(19) United States
(12) Patent Application Publication
(10) Pub. No.: US 2024/0172559 A1
(43) Pub. Date: May 23, 2024

(54) ORGANIC LIGHT EMITTING DEVICE

(71) Applicant: LG CHEM, LTD., Seoul (KR)

(72) Inventors: Minjun KIM, Daejeon (KR); Dong Hoon LEE, Daejeon (KR); Sang Duk SUH, Daejeon (KR); Young Seok KIM, Daejeon (KR)

(21) Appl. No.: 18/272,344

(22) PCT Filed: Jul. 22, 2022
(86) PCT No.: PCT/KR2022/010823

§ 371 Date: Jul. 13, 2023

(30) Foreign Application Priority Data
Jul. 22, 2021 (KR) 10-2021-0096453
Jul. 22, 2022 (KR) 10-2022-0091005

(51) Int. Cl. H10K 85/60; C07D 307/77; C07D 307/91; C07D 405/04; C07D 405/10; C07D 405/14; C07D 409/12; C07D 409/14; C07F 7/08; H10K 85/40

(52) U.S. Cl. CPC H10K 85/654; C07D 307/77; C07D 307/91; C07D 405/04; C07D 405/10; C07D 405/14; C07D 409/12; C07D 409/14; C07F 7/0812; H10K 85/40; H10K 85/615; H10K 85/622; H10K 85/626; H10K 85/633; H10K 85/636; H10K 85/6574; H10K 85/6576; H10K 50/11

(57) ABSTRACT

An organic light emitting device comprising an anode, a cathode, and a light emitting layer between the anode and the cathode, the light emitting layer including a compound represented by Chemical Formula 1 and a compound represented by Chemical Formula 2, is provided.

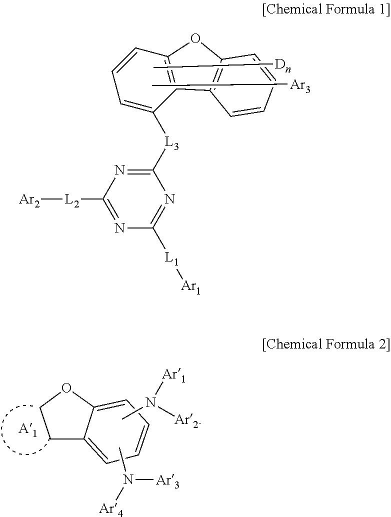

### CROSS-REFERENCE TO RELATED APPLICATION(S)

This application is a National Phase entry pursuant to 35 U.S.C. § 371 of International Application No. PCT/KR2022/010823 filed on Jul. 22, 2022, and claims priority to and the benefit of Korean Patent Application No. 10-2021-0096453 filed on Jul. 22, 2021 and Korean Patent Application No. 10-2022-0091005 filed on Jul. 22, 2022, the disclosures of which are incorporated herein by reference in their entirety.

### FIELD OF DISCLOSURE

The present disclosure relates to an organic light emitting device.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer between the anode and the cathode. The organic material layer frequently has a multilayered structure that comprises different materials in order to enhance efficiency and stability of the organic light emitting device, and for example, the organic material layer may be formed of a hole injection layer, a hole transport layer, a light emitting layer, an electron transport layer, an electron injection layer and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuing need for the development of a new material for an organic material used in the organic light emitting device as described above.

### RELATED ART

Korean Unexamined Patent Publication No. 10-2000-0051826

### SUMMARY

It is an object of the present disclosure to provide an organic light emitting device having improved driving voltage, efficiency and lifetime.

In order to achieve the above object, according to the present disclosure, there is provided an organic light emitting device comprising:
an anode; a cathode; and a light emitting layer between the anode and the cathode,
wherein the light emitting layer includes a compound represented by the following Chemical Formula 1 and a compound represented by the following Chemical Formula 2.

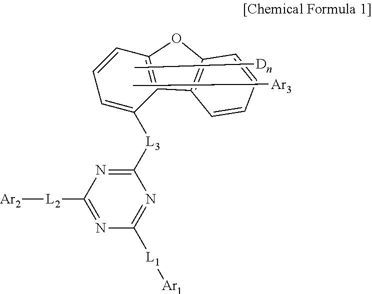

in Chemical Formula 1,
L₁ to L₃ are a single bond; or a substituted or unsubstituted C₆₋₆₀ arylene,
Ar₁ and Ar₂ are each independently a substituted or unsubstituted C₆₋₆₀ aryl; or a substituted or unsubstituted C₂₋₆₀ heteroaryl containing one or more selected from the group consisting of N, O and S,
Ar₃ is hydrogen; deuterium; a substituted or unsubstituted C₆₋₆₀ aryl; or a substituted or unsubstituted C₂₋₆₀ heteroaryl containing one or more selected from the group consisting of N, O and S,
D is deuterium, and
n is an integer of 0 to 6,

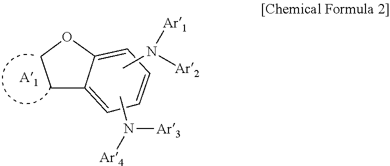

in Chemical Formula 2,
A′₁ is a naphthalene ring, and
Ar′₁ to Ar′₄ are each independently a substituted or unsubstituted C₆₋₆₀ aryl; or a substituted or unsubstituted C₂₋₆₀ heteroaryl containing one or more heteroatoms selected from the group consisting of N, O and S.

The above-mentioned organic light emitting device is excellent in driving voltage, efficiency and lifetime.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, a light emitting layer 3, an electron transport layer 7, an electron injection layer 8 and a cathode 4.

FIG. 3 shows an example of an organic light emitting device comprising a substrate 1, an anode 2, a hole injection layer 5, a hole transport layer 6, an electron blocking layer 9, a light emitting layer 3, a hole blocking layer 10, an electron injection and transport layer 11, and a cathode 4.

### DETAILED DESCRIPTION

Hereinafter, embodiments of the present disclosure will be described in more detail to facilitate understanding of the invention.

As used herein, the notation [symbol] or [symbol] means a bond linked to another substituent group.

As used herein, the term “substituted or unsubstituted” means being unsubstituted or substituted with one or more substituents selected from the substituent group consisting of deuterium; a halogen group; a nitrile group; a nitro group; a hydroxy group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; and a heterocyclic group containing one or more of N, O and S atoms, or being unsubstituted or substituted with a substituent from the above substituent group which is further substituted by one or more selected from the above substituent group.

In the present disclosure, the carbon number of a carbonyl group is not particularly limited, but is preferably 1 to 40. A specific example thereof may be a compound having the following structural formulas, but is not limited thereto.

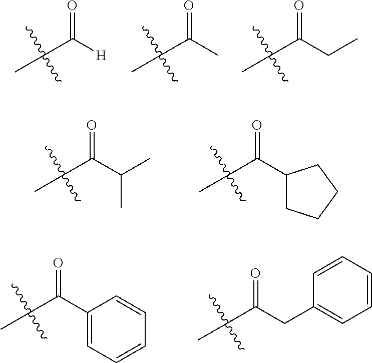

In the present disclosure, an ester group may have a structure in which oxygen of the ester group may be substituted by a straight-chain, branched-chain, or cyclic alkyl group having 1 to 25 carbon atoms, or an aryl group having 6 to 25 carbon atoms. A specific example thereof may be a compound having the following structural formulas, but is not limited thereto.

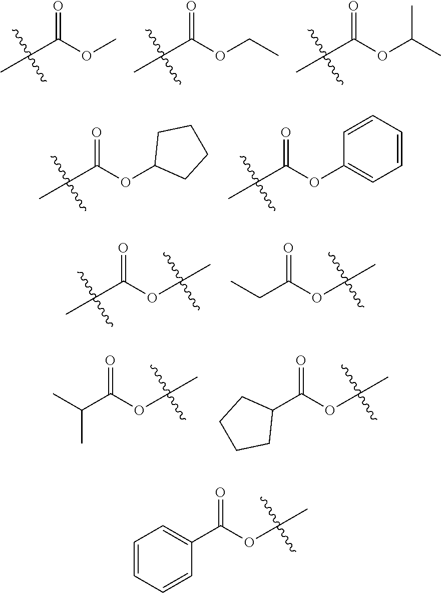

In the present disclosure, the carbon number of an imide group is not particularly limited, but is preferably 1 to 25. A specific example thereof may be a compound having the following structural formulas, but is not limited thereto.

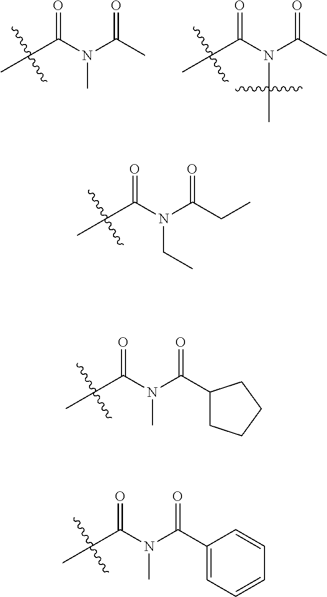

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but is not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group may be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to yet another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group may be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it may be a monocyclic aryl group or a polycyclic aryl group. According to one embodiment, the carbon number of the aryl group is 6 to 30. According to one embodiment, the carbon number of the aryl group is 6 to 20. The aryl group may be a phenyl group, a biphenyl group, a terphenyl group or the like as the monocyclic aryl group, but is not limited thereto. The polycyclic aryl group includes a naphthyl group, an anthracenyl group, a phenanthrenyl group, a pyrenyl group, a perylenyl group, a chrysenyl group, a fluorenyl group, and the like, but is not limited thereto.

In the present disclosure, the fluorenyl group may be substituted, and two substituents may be linked with each other to form a spiro structure. In the case where the fluorenyl group is substituted,

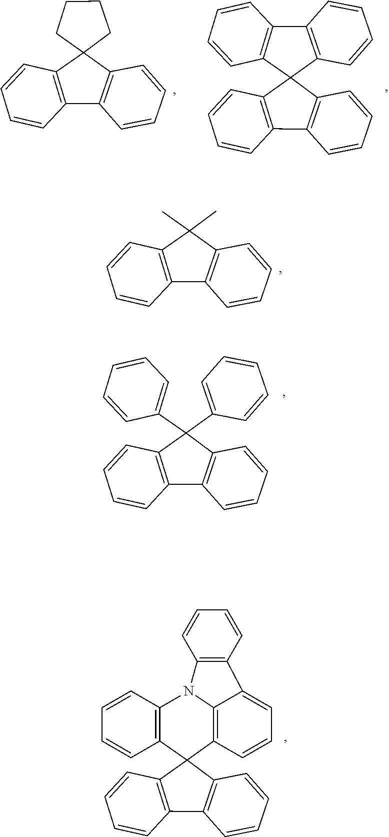

and the like can be formed. However, the structure is not limited thereto.

In the present disclosure, a heterocyclic group is a heterocyclic group containing one or more of O, N, Si and S as a heteroatom, and the carbon number thereof is not particularly limited, but is preferably 2 to 60. Examples of the heterocyclic group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group and the arylamine group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine can be applied to the aforementioned description of the heterocyclic group. In the present disclosure, the alkenyl group in the aralkenyl group is the same as the aforementioned examples of the alkenyl group. In the present disclosure, the aforementioned description of the aryl group may be applied except that the arylene is a divalent group. In the present disclosure, the aforementioned description of the heterocyclic group can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heterocyclic group can be applied, except that the heterocycle is not a monovalent group but formed by combining two substituent groups.

Hereinafter, the present disclosure will be described in detail for each configuration.

Anode and Cathode

An anode and a cathode used in the present disclosure mean electrodes used in an organic light emitting device.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

Light Emitting Layer

The light emitting layer used in the present disclosure is a layer that can emit light in the visible light region by combining holes and electrons transported from the anode and the cathode. Generally, the light emitting layer includes a host material and a dopant material, and in the present disclosure, the compound represented by Chemical Formula 1 and the compound represented by Chemical Formula 2 are included as a host.

The compound of Chemical Formula 1 has a structure containing a triazine substituent at the 1-position of the dibenzofuran core.

Preferably, L₃ is a single bond; or a substituted or unsubstituted C₆₋₂₀ arylene. More preferably, L₃ is a single bond; phenylene; or naphthalenediyl.

Preferably, L₁ and L₂ are each independently a single bond; or a substituted or unsubstituted C₆₋₂₀ arylene. More preferably, L₁ and L₂ are each independently a single bond; phenylene unsubstituted or substituted with one or more deuteriums; biphenyldiyl unsubstituted or substituted with one or more deuteriums; or naphthalenediyl unsubstituted or substituted with one or more deuteriums.

Preferably, Ar₁ and Ar₂ are each independently a substituted or unsubstituted C₆₋₂₀ aryl; or a substituted or unsubstituted C₂₋₂₀ heteroaryl containing any one or more heteroatoms selected from the group consisting of N, O and S.

More preferably, Ar₁ and Ar₂ are each independently phenyl unsubstituted or substituted with one or more deuteriums; phenyl substituted with triphenylsilyl; biphenylyl unsubstituted or substituted with one or more deuteriums; terphenylyl unsubstituted or substituted with one or more deuteriums; naphthyl unsubstituted or substituted with one or more deuteriums; phenanthrenyl unsubstituted or substituted with one or more deuteriums; dibenzofuranyl unsubstituted or substituted with one or more deuteriums; or dibenzothiophenyl unsubstituted or substituted with one or more deuteriums.

Preferably, Ar₃ is hydrogen; deuterium; a substituted or unsubstituted C₆₋₂₀ aryl; or a substituted or unsubstituted C₂₋₂₀ heteroaryl containing one or more heteroatoms selected from the group consisting of N, O and S.

More preferably, Ar₃ is hydrogen; deuterium; phenyl unsubstituted or substituted with one or more deuteriums; biphenylyl unsubstituted or substituted with one or more deuteriums; terphenylyl unsubstituted or substituted with one or more deuteriums; naphthyl unsubstituted or substituted with one or more deuteriums; phenanthrenyl unsubstituted or substituted with one or more deuteriums; fluoranthenyl unsubstituted or substituted with one or more deuteriums; phenylnaphthyl unsubstituted or substituted with one or more deuteriums; naphthylphenyl unsubstituted or substituted with one or more deuteriums; triphenylenyl unsubstituted or substituted with one or more deuteriums; dibenzofuranyl unsubstituted or substituted with one or more deuteriums; dibenzothiophenyl unsubstituted or substituted with one or more deuteriums; benzonaphthofuranyl unsubstituted or substituted with one or more deuteriums; or benzonaphthothiophenyl unsubstituted or substituted with one or more deuteriums.

Further, the compound of Chemical Formula 1 may not contain any deuterium, or may contain one or more deuteriums.

In one example, when the compound contains deuterium, the deuterium substitution rate of the compound may be 1% to 100%. Specifically, the deuterium substitution rate of the compound may be 5% or more, 10% or more, 20% or more, 30% or more, 40% or more, 50% or more, 60% or more, 70% or more, 75% or more, 80% or more, or 90% or more, and less than 100%. The deuterium substitution rate of such a compound is calculated as the number of substituted deuterium relative to the total number of hydrogens that can be present in the Chemical Formula, wherein the number of substituted deuterium may be obtained through MALDI-TOF MS (Matrix-Assisted Laser Desorption/Ionization Time-of-Flight Mass Spectrometer) analysis.

Representative examples of Chemical Formula 1 are as follows:

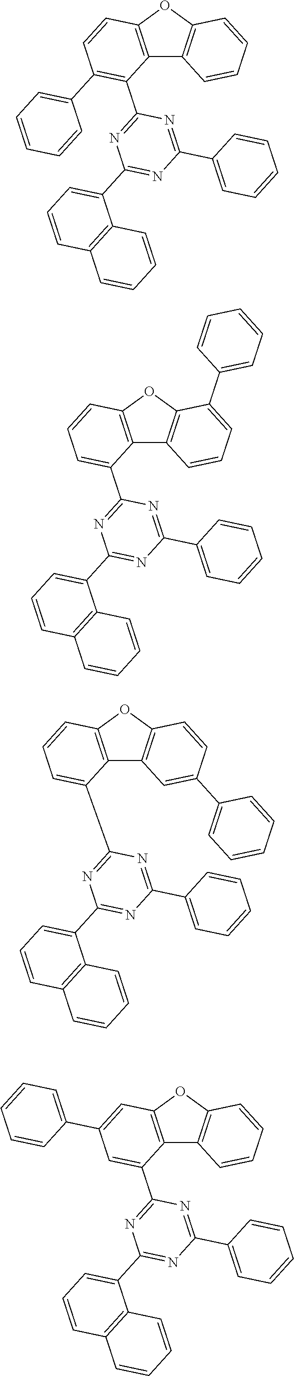

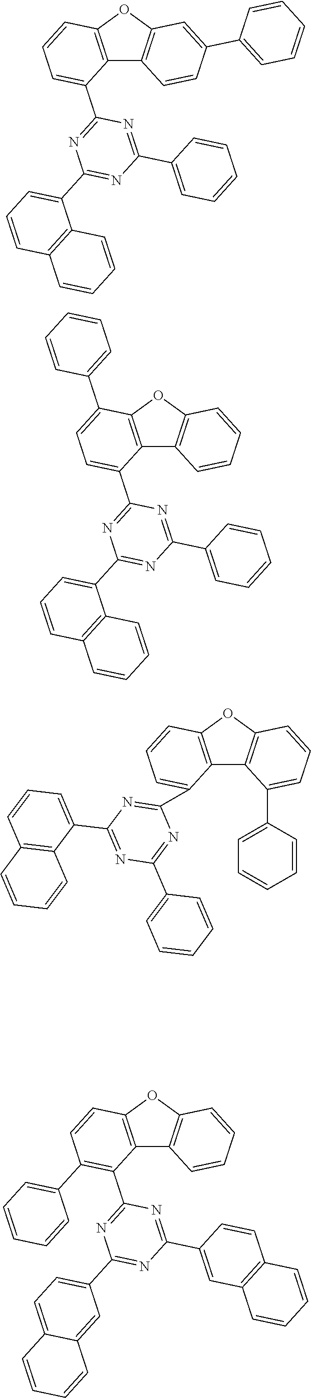

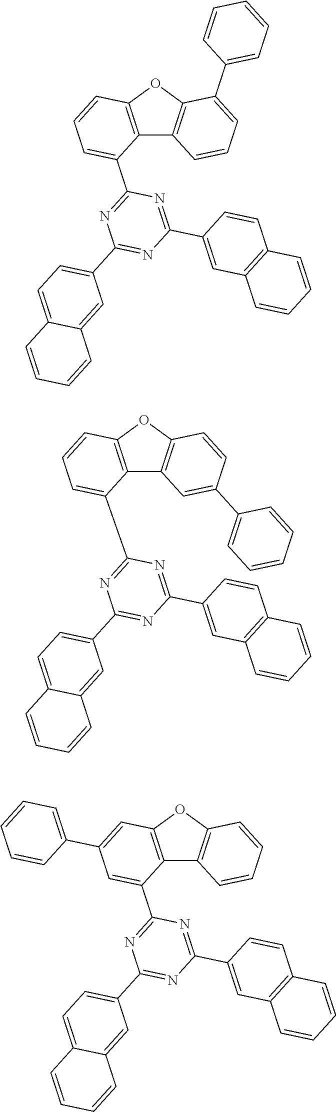 [SMILES: c1ccc(-c2cccc3c2oc2cccc(-c4nc(-c5ccc6ccccc6c5)nc(-c5ccc6ccccc6c5)n4)c23)cc1 ; c1ccc(-c2ccc3oc4cccc(-c5nc(-c6ccc7ccccc7c6)nc(-c6ccc7ccccc7c6)n5)c4c3c2)cc1 ; c1ccc(-c2cc(-c3nc(-c4ccc5ccccc5c4)nc(-c4ccc5ccccc5c4)n3)c3c(c2)oc2ccccc23)cc1]

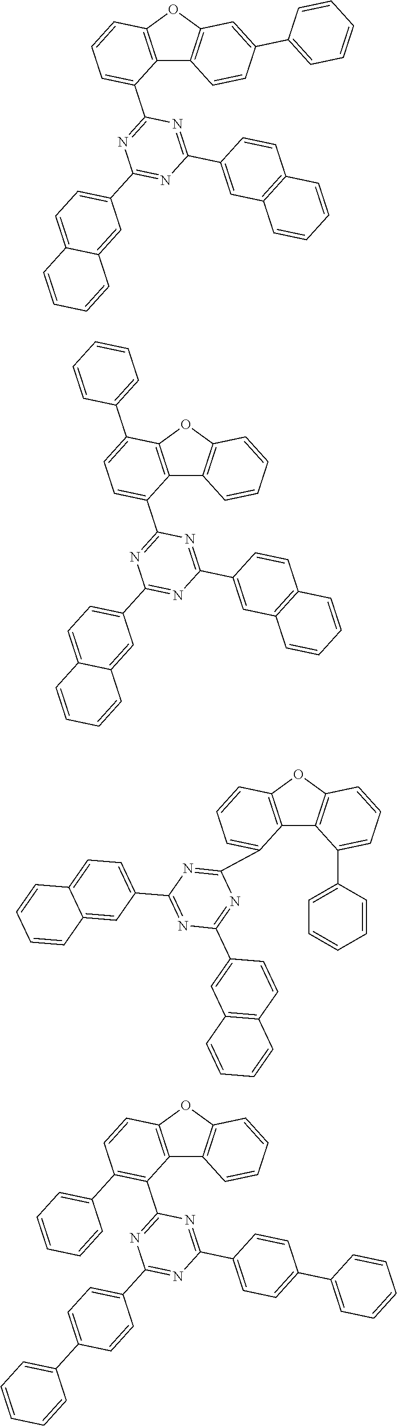

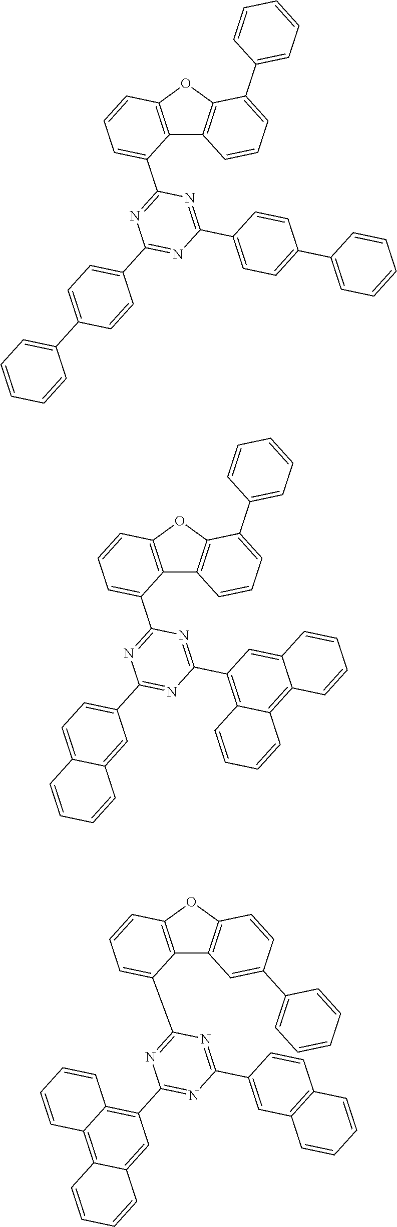 [SMILES: c1ccc(-c2ccc(-c3nc(-c4ccc(-c5ccccc5)cc4)nc(-c4cccc5oc6c(-c7ccccc7)cccc6c45)n3)cc2)cc1 ; c1ccc(-c2cccc3c2oc2cccc(-c4nc(-c5ccc6ccccc6c5)nc(-c5cc6ccccc6c6ccccc56)n4)c23)cc1 ; c1ccc(-c2ccc3oc4cccc(-c5nc(-c6ccc7ccccc7c6)nc(-c6cc7ccccc7c7ccccc67)n5)c4c3c2)cc1]

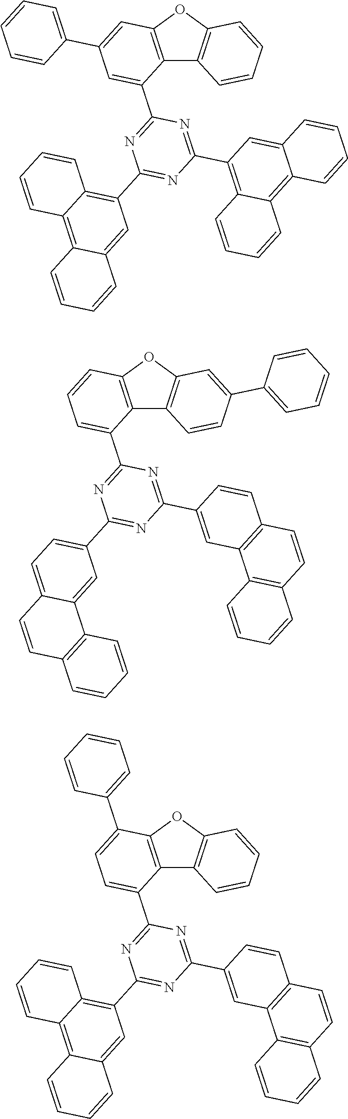

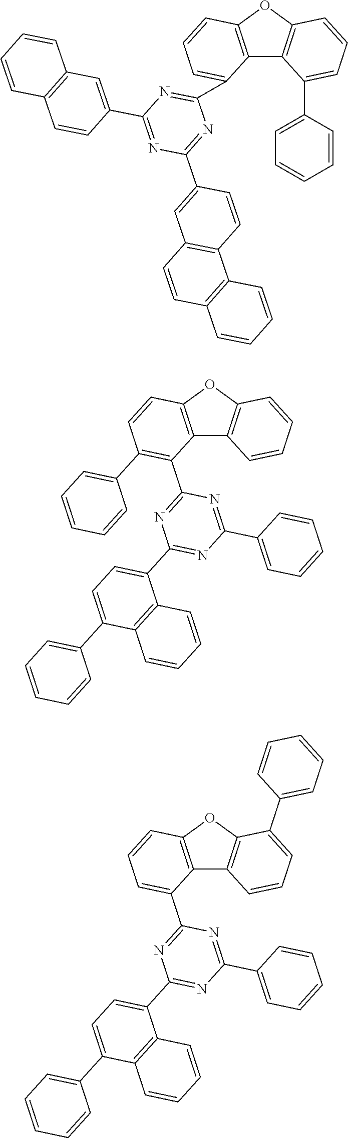 [SMILES: c1ccc(-c2cccc3oc4cccc(-c5nc(-c6ccc7ccccc7c6)nc(-c6ccc7c(ccc8ccccc87)c6)n5)c4c23)cc1 ; c1ccc(-c2nc(-c3ccc(-c4ccccc4)c4ccccc34)nc(-c3c(-c4ccccc4)ccc4oc5ccccc5c34)n2)cc1 ; c1ccc(-c2nc(-c3ccc(-c4ccccc4)c4ccccc34)nc(-c3cccc4oc5c(-c6ccccc6)cccc5c34)n2)cc1]

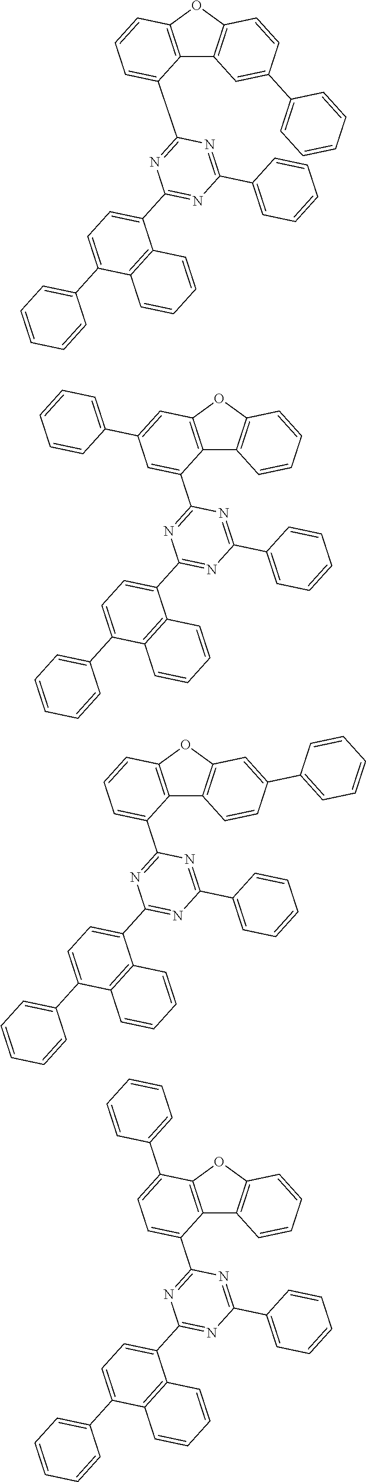

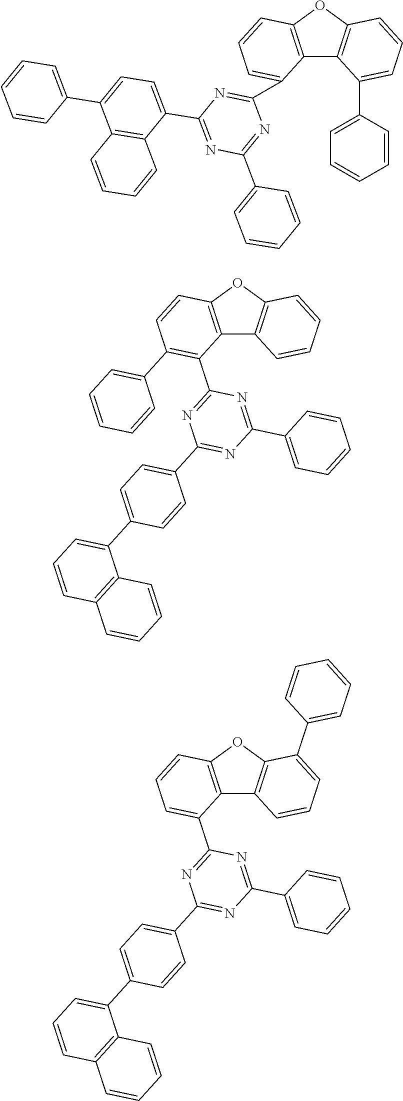

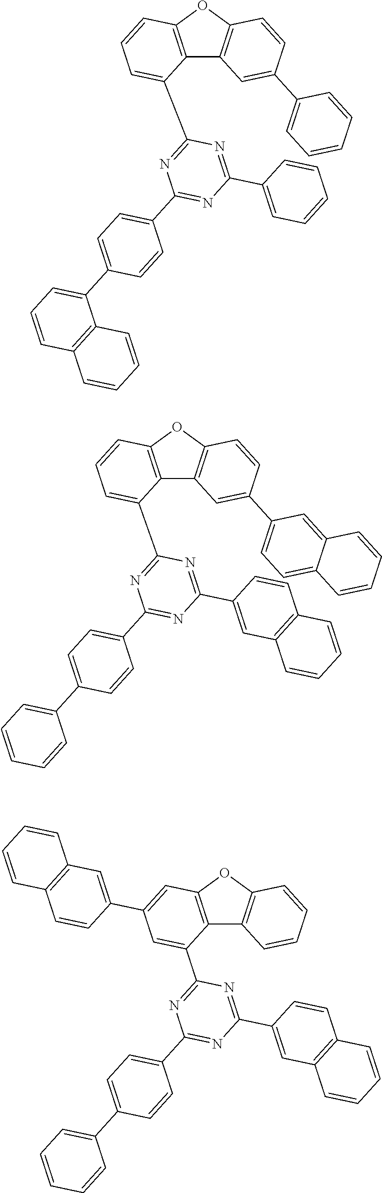 [SMILES: c1ccc(-c2ccc3oc4cccc(-c5nc(-c6ccccc6)nc(-c6ccc(-c7cccc8ccccc78)cc6)n5)c4c3c2)cc1 ; c1ccc(-c2ccc(-c3nc(-c4ccc5ccccc5c4)nc(-c4cccc5oc6ccc(-c7ccc8ccccc8c7)cc6c45)n3)cc2)cc1 ; c1ccc(-c2ccc(-c3nc(-c4ccc5ccccc5c4)nc(-c4cc(-c5ccc6ccccc6c5)cc5oc6ccccc6c45)n3)cc2)cc1]

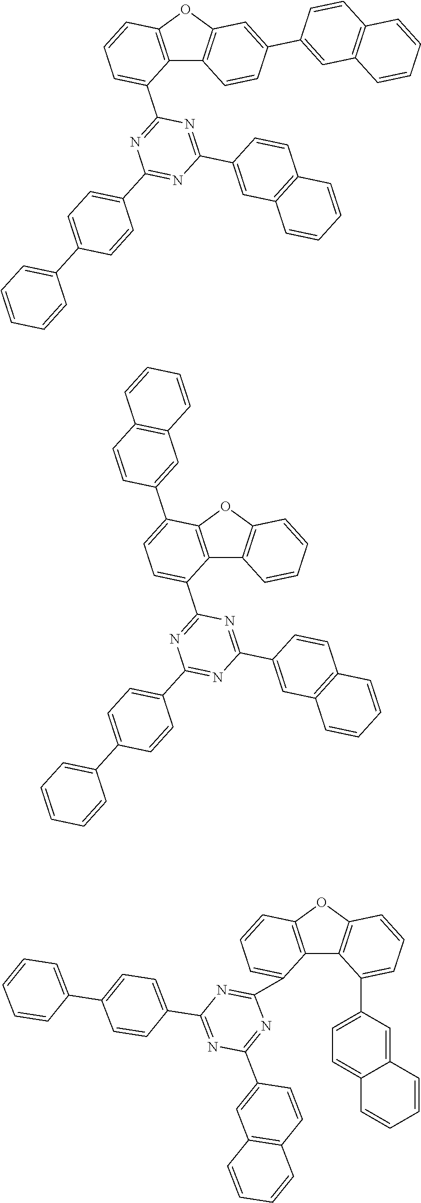 [SMILES: c1ccc(-c2ccc(-c3nc(-c4ccc5ccccc5c4)nc(-c4cccc5oc6cc(-c7ccc8ccccc8c7)ccc6c45)n3)cc2)cc1 ; c1ccc(-c2ccc(-c3nc(-c4ccc5ccccc5c4)nc(-c4ccc(-c5ccc6ccccc6c5)c5oc6ccccc6c45)n3)cc2)cc1 ; c1ccc(-c2ccc(-c3nc(-c4ccc5ccccc5c4)nc(-c4cccc5oc6cccc(-c7ccc8ccccc8c7)c6c45)n3)cc2)cc1]

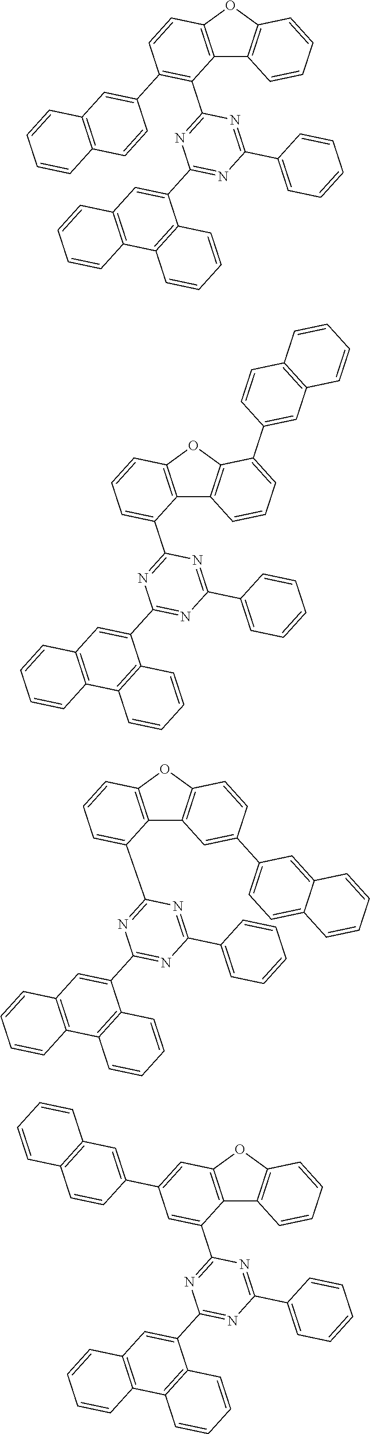

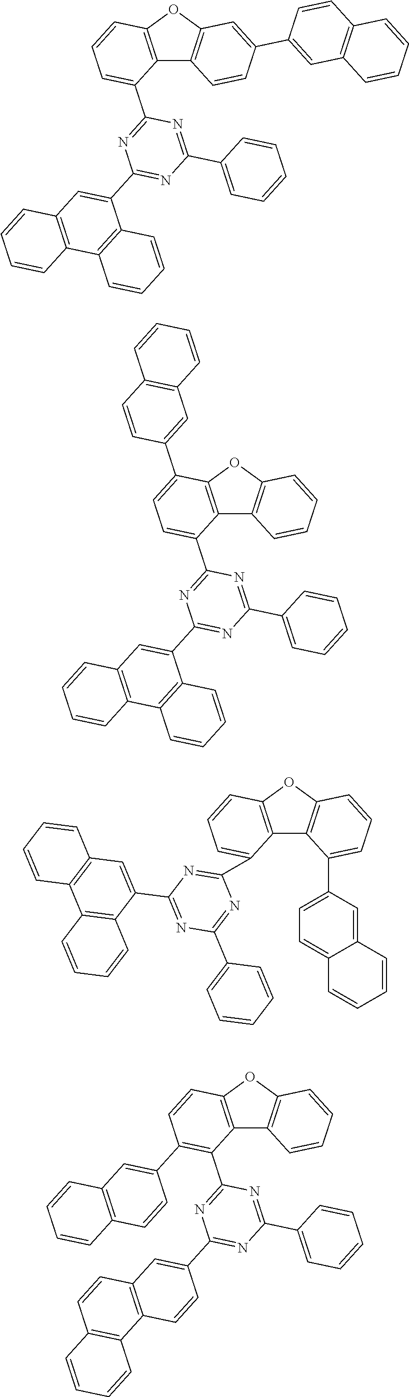

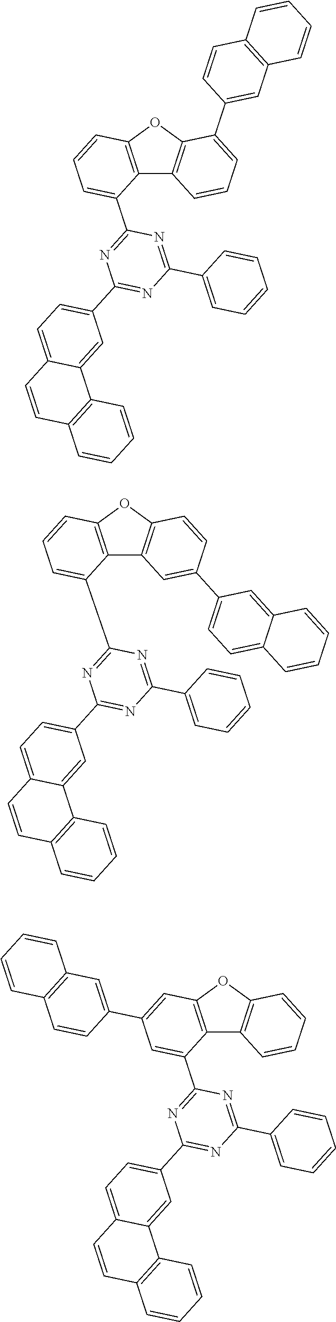

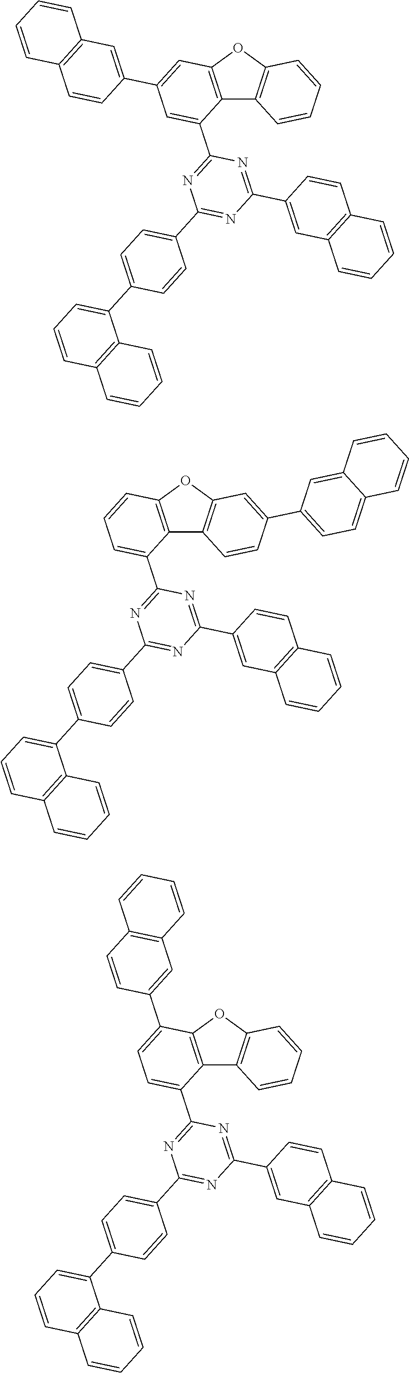 [SMILES: c1ccc2cc(-c3cc(-c4nc(-c5ccc(-c6cccc7ccccc67)cc5)nc(-c5ccc6ccccc6c5)n4)c4c(c3)oc3ccccc34)ccc2c1 ; c1ccc2cc(-c3ccc4c(c3)oc3cccc(-c5nc(-c6ccc(-c7cccc8ccccc78)cc6)nc(-c6ccc7ccccc7c6)n5)c34)ccc2c1 ; c1ccc2cc(-c3nc(-c4ccc(-c5cccc6ccccc56)cc4)nc(-c4ccc(-c5ccc6ccccc6c5)c5oc6ccccc6c45)n3)ccc2c1]

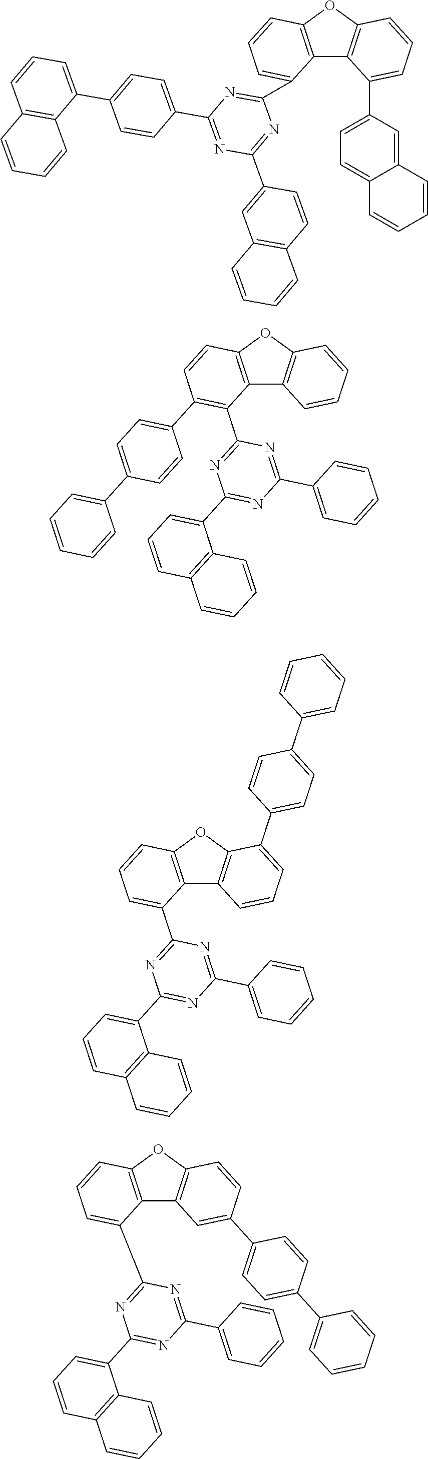

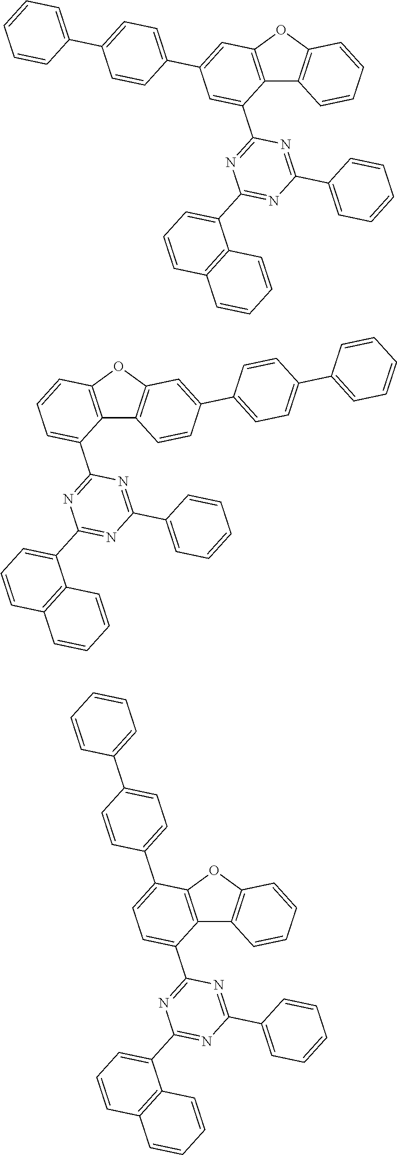

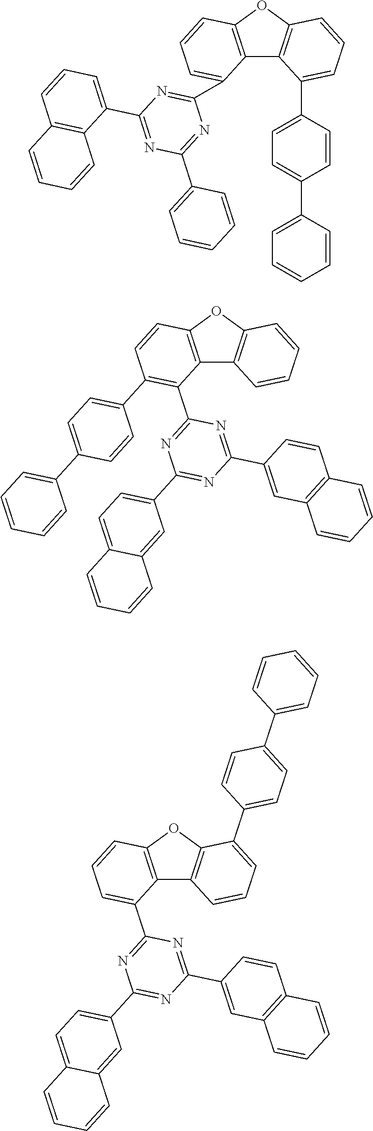 [SMILES: c1ccc(-c2ccc(-c3cccc4oc5cccc(-c6nc(-c7ccccc7)nc(-c7cccc8ccccc78)n6)c5c34)cc2)cc1 ; c1ccc(-c2ccc(-c3ccc4oc5ccccc5c4c3-c3nc(-c4ccc5ccccc5c4)nc(-c4ccc5ccccc5c4)n3)cc2)cc1 ; c1ccc(-c2ccc(-c3cccc4c3oc3cccc(-c5nc(-c6ccc7ccccc7c6)nc(-c6ccc7ccccc7c6)n5)c34)cc2)cc1]

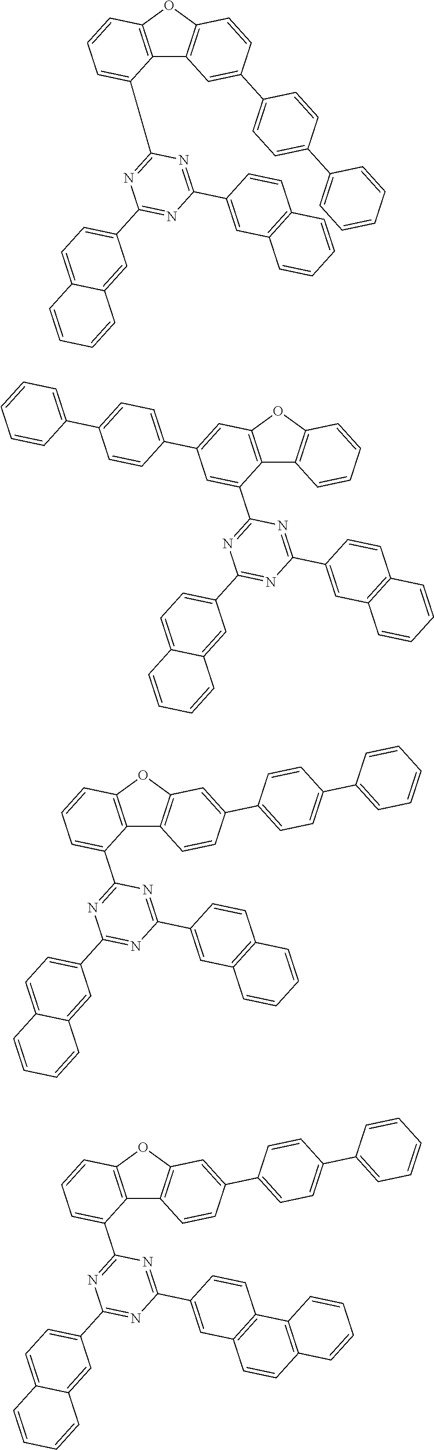 [SMILES: c1ccc(-c2ccc(-c3ccc4oc5cccc(-c6nc(-c7ccc8ccccc8c7)nc(-c7ccc8ccccc8c7)n6)c5c4c3)cc2)cc1 ; c1ccc(-c2ccc(-c3cc(-c4nc(-c5ccc6ccccc6c5)nc(-c5ccc6ccccc6c5)n4)c4c(c3)oc3ccccc34)cc2)cc1 ; c1ccc(-c2ccc(-c3ccc4c(c3)oc3cccc(-c5nc(-c6ccc7ccccc7c6)nc(-c6ccc7ccccc7c6)n5)c34)cc2)cc1 ; c1ccc(-c2ccc(-c3ccc4c(c3)oc3cccc(-c5nc(-c6ccc7ccccc7c6)nc(-c6ccc7c(ccc8ccccc87)c6)n5)c34)cc2)cc1]

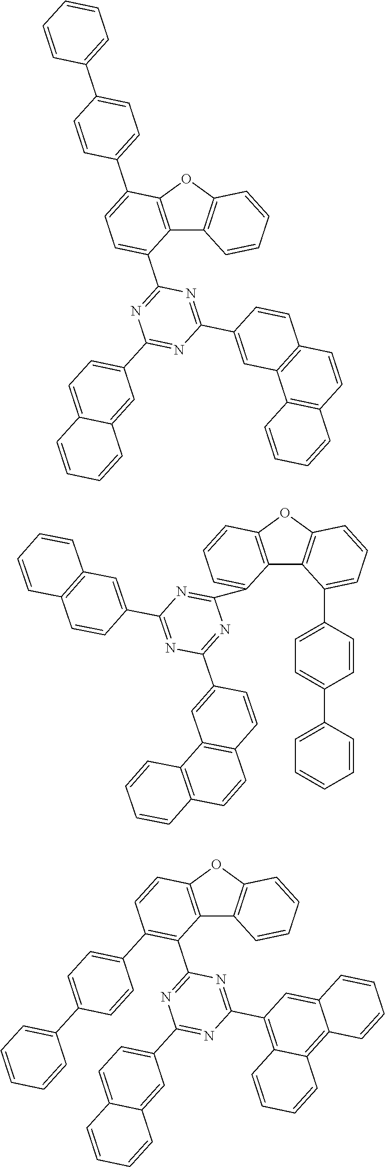 [SMILES: c1ccc(-c2ccc(-c3ccc(-c4nc(-c5ccc6ccccc6c5)nc(-c5ccc6ccc7ccccc7c6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccc9ccccc9c8c7)n6)c5c34)cc2)cc1 ; c1ccc(-c2ccc(-c3ccc4oc5ccccc5c4c3-c3nc(-c4ccc5ccccc5c4)nc(-c4cc5ccccc5c5ccccc45)n3)cc2)cc1]

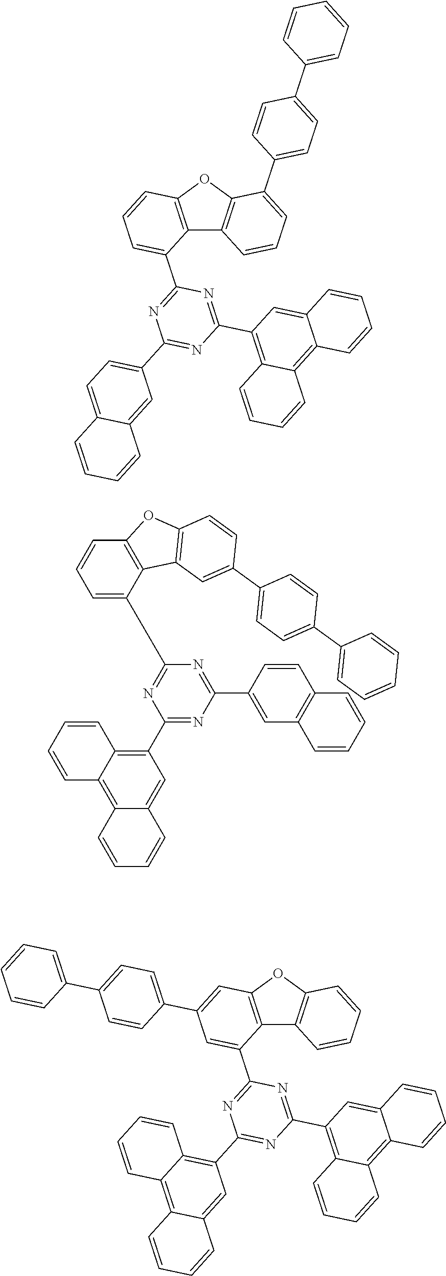

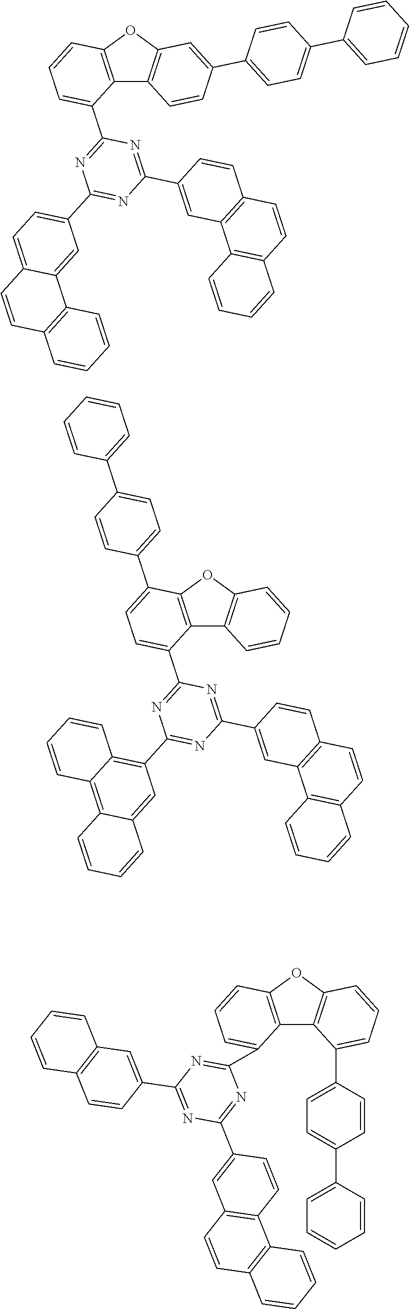

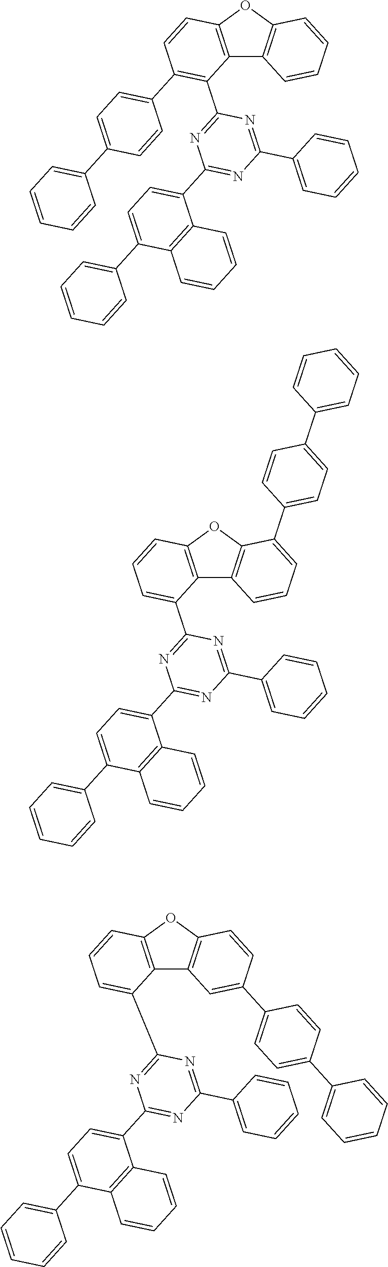 [SMILES: c1ccc(-c2ccc(-c3ccc4oc5ccccc5c4c3-c3nc(-c4ccccc4)nc(-c4ccc(-c5ccccc5)c5ccccc45)n3)cc2)cc1 ; c1ccc(-c2ccc(-c3cccc4c3oc3cccc(-c5nc(-c6ccccc6)nc(-c6ccc(-c7ccccc7)c7ccccc67)n5)c34)cc2)cc1 ; c1ccc(-c2ccc(-c3ccc4oc5cccc(-c6nc(-c7ccccc7)nc(-c7ccc(-c8ccccc8)c8ccccc78)n6)c5c4c3)cc2)cc1]

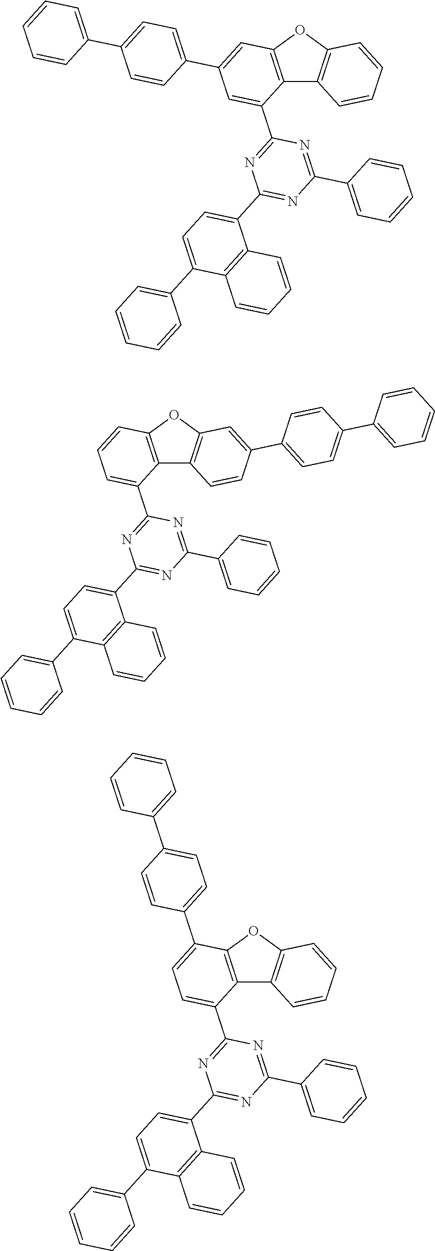 [SMILES: c1ccc(-c2ccc(-c3cc(-c4nc(-c5ccccc5)nc(-c5ccc(-c6ccccc6)c6ccccc56)n4)c4c(c3)oc3ccccc34)cc2)cc1 ; c1ccc(-c2ccc(-c3ccc4c(c3)oc3cccc(-c5nc(-c6ccccc6)nc(-c6ccc(-c7ccccc7)c7ccccc67)n5)c34)cc2)cc1 ; c1ccc(-c2ccc(-c3ccc(-c4nc(-c5ccccc5)nc(-c5ccc(-c6ccccc6)c6ccccc56)n4)c4c3oc3ccccc34)cc2)cc1]

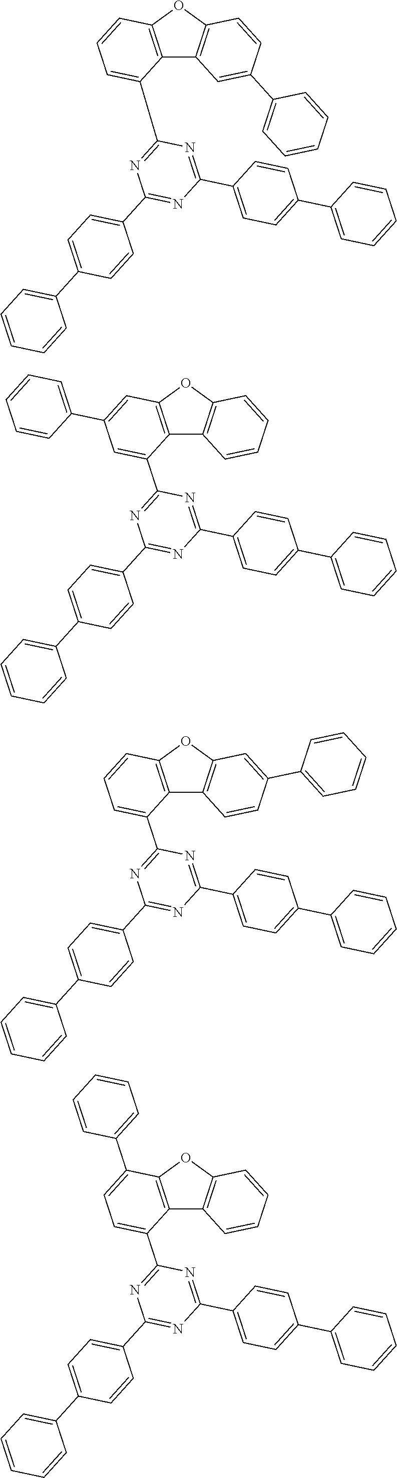

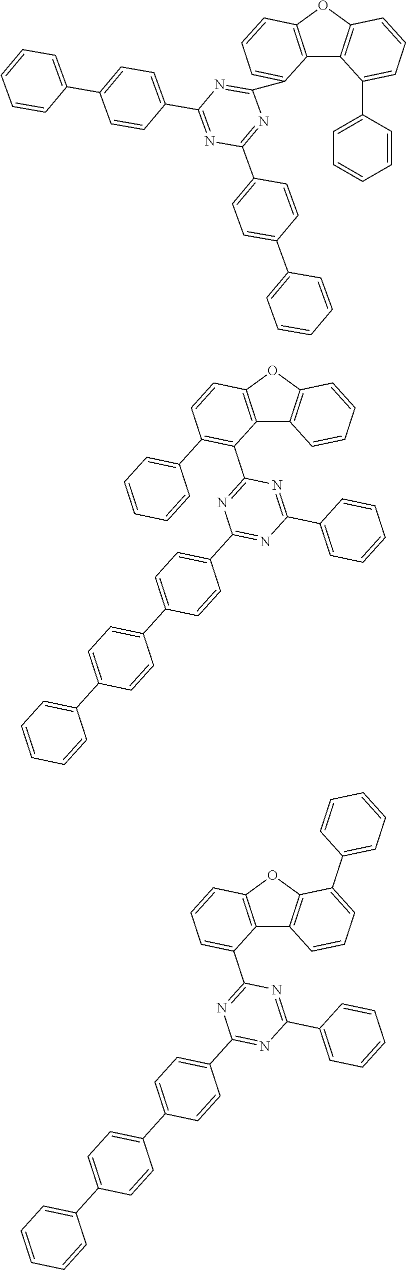

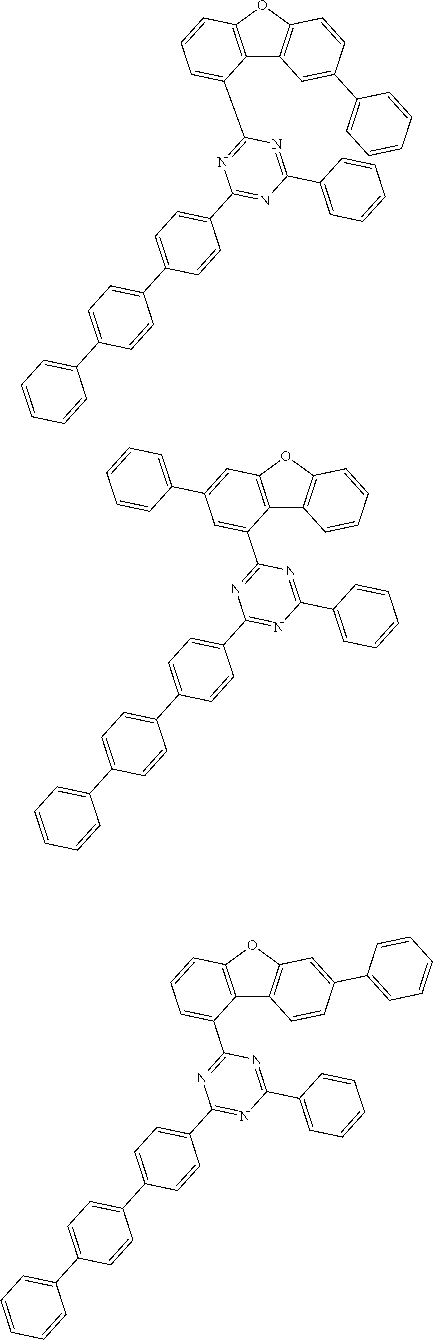 [SMILES: c1ccc(-c2ccc(-c3ccc(-c4nc(-c5ccccc5)nc(-c5cccc6oc7ccc(-c8ccccc8)cc7c56)n4)cc3)cc2)cc1 ; c1ccc(-c2ccc(-c3ccc(-c4nc(-c5ccccc5)nc(-c5cc(-c6ccccc6)cc6oc7ccccc7c56)n4)cc3)cc2)cc1 ; c1ccc(-c2ccc(-c3ccc(-c4nc(-c5ccccc5)nc(-c5cccc6oc7cc(-c8ccccc8)ccc7c56)n4)cc3)cc2)cc1]

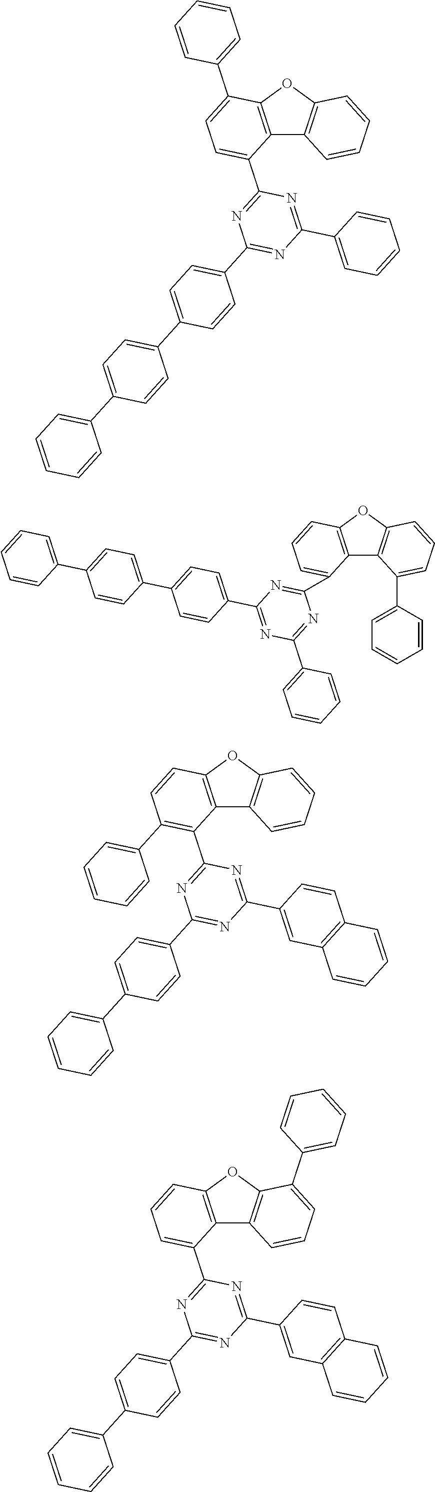 [SMILES: c1ccc(-c2ccc(-c3ccc(-c4nc(-c5ccccc5)nc(-c5ccc(-c6ccccc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccccc8)c7c56)n4)cc3)cc2)cc1 ; c1ccc(-c2ccc(-c3nc(-c4ccc5ccccc5c4)nc(-c4c(-c5ccccc5)ccc5oc6ccccc6c45)n3)cc2)cc1 ; c1ccc(-c2ccc(-c3nc(-c4ccc5ccccc5c4)nc(-c4cccc5oc6c(-c7ccccc7)cccc6c45)n3)cc2)cc1]

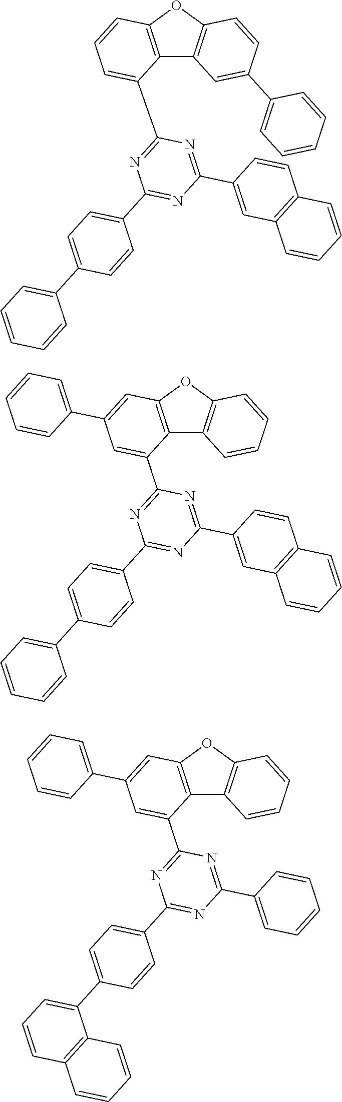

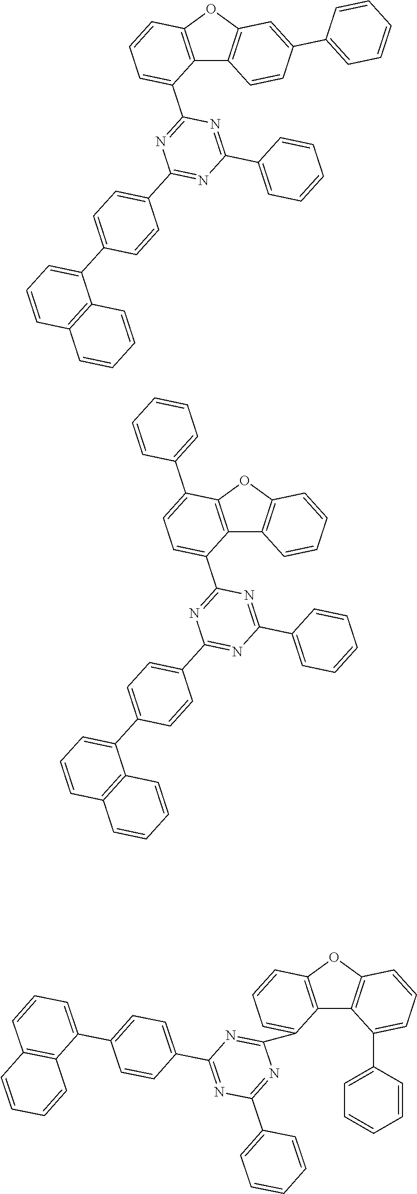 [SMILES: c1ccc(-c2ccc3c(c2)oc2cccc(-c4nc(-c5ccccc5)nc(-c5ccc(-c6cccc7ccccc67)cc5)n4)c23)cc1 ; c1ccc(-c2nc(-c3ccc(-c4cccc5ccccc45)cc3)nc(-c3ccc(-c4ccccc4)c4oc5ccccc5c34)n2)cc1 ; c1ccc(-c2nc(-c3ccc(-c4cccc5ccccc45)cc3)nc(-c3cccc4oc5cccc(-c6ccccc6)c5c34)n2)cc1]

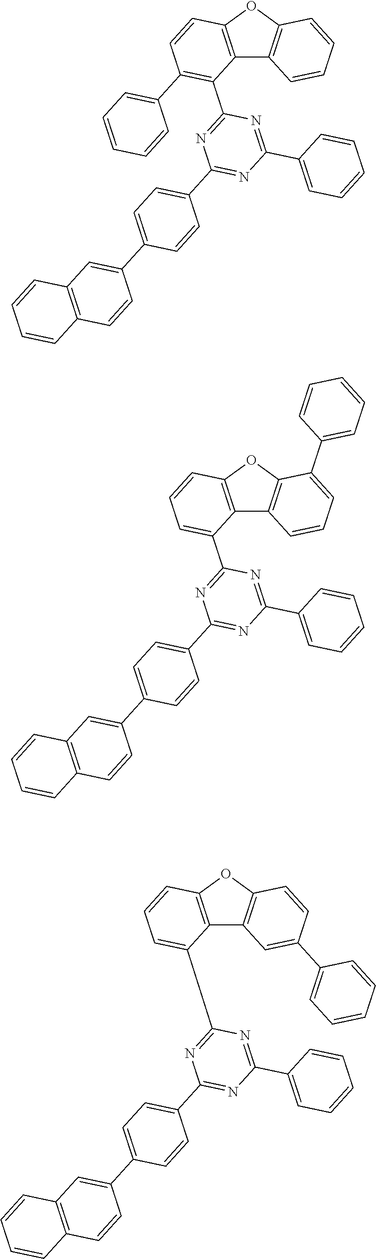 [SMILES: c1ccc(-c2nc(-c3ccc(-c4ccc5ccccc5c4)cc3)nc(-c3c(-c4ccccc4)ccc4oc5ccccc5c34)n2)cc1 ; c1ccc(-c2nc(-c3ccc(-c4ccc5ccccc5c4)cc3)nc(-c3cccc4oc5c(-c6ccccc6)cccc5c34)n2)cc1 ; c1ccc(-c2ccc3oc4cccc(-c5nc(-c6ccccc6)nc(-c6ccc(-c7ccc8ccccc8c7)cc6)n5)c4c3c2)cc1]

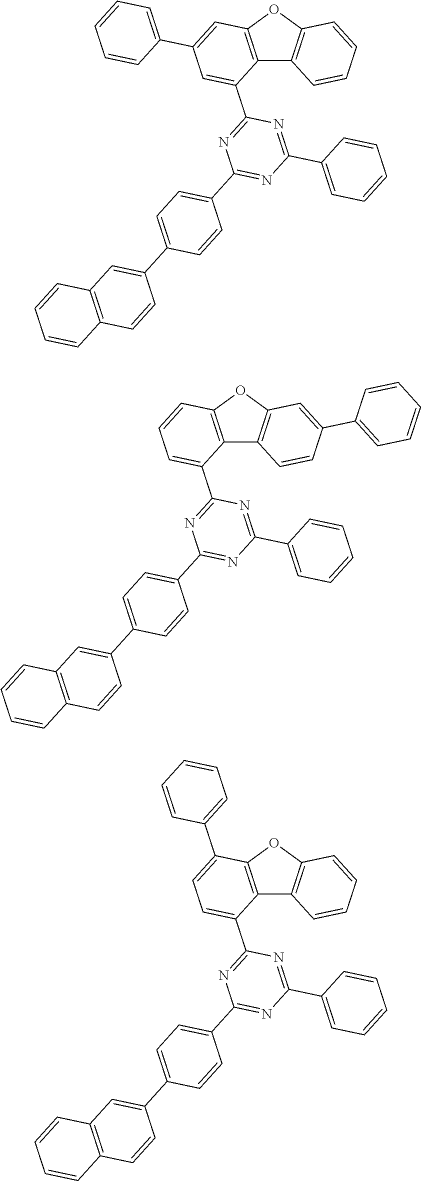 [SMILES: c1ccc(-c2cc(-c3nc(-c4ccccc4)nc(-c4ccc(-c5ccc6ccccc6c5)cc4)n3)c3c(c2)oc2ccccc23)cc1 ; c1ccc(-c2ccc3c(c2)oc2cccc(-c4nc(-c5ccccc5)nc(-c5ccc(-c6ccc7ccccc7c6)cc5)n4)c23)cc1 ; c1ccc(-c2nc(-c3ccc(-c4ccc5ccccc5c4)cc3)nc(-c3ccc(-c4ccccc4)c4oc5ccccc5c34)n2)cc1]

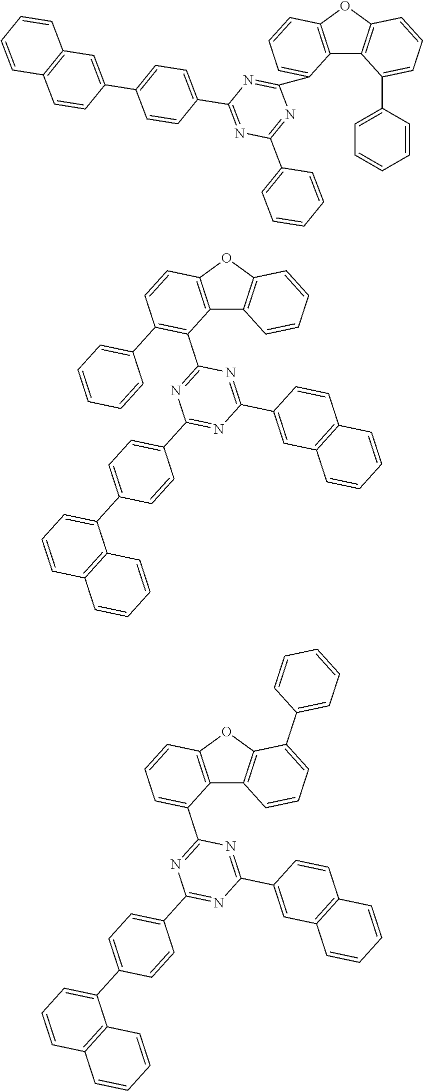 [SMILES: c1ccc(-c2nc(-c3ccc(-c4ccc5ccccc5c4)cc3)nc(-c3cccc4oc5cccc(-c6ccccc6)c5c34)n2)cc1 ; c1ccc(-c2ccc3oc4ccccc4c3c2-c2nc(-c3ccc(-c4cccc5ccccc45)cc3)nc(-c3ccc4ccccc4c3)n2)cc1 ; c1ccc(-c2cccc3c2oc2cccc(-c4nc(-c5ccc(-c6cccc7ccccc67)cc5)nc(-c5ccc6ccccc6c5)n4)c23)cc1]

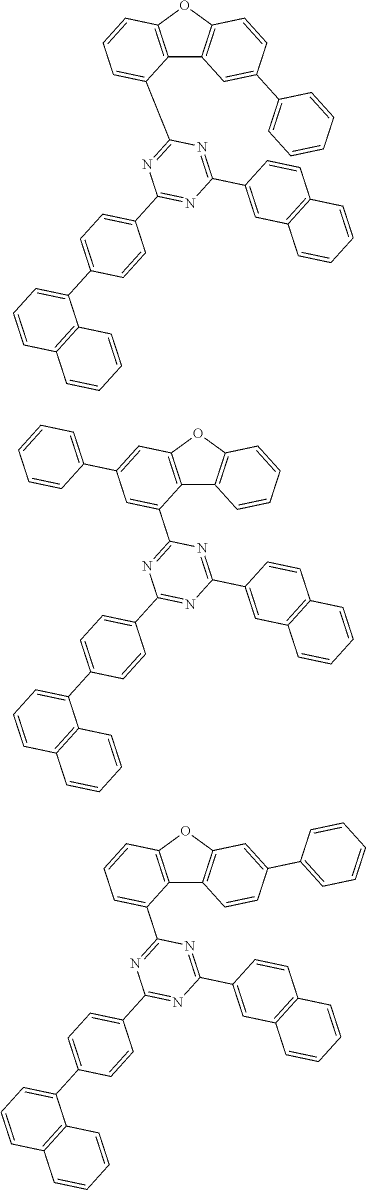 [SMILES: c1ccc(-c2ccc3oc4cccc(-c5nc(-c6ccc(-c7cccc8ccccc78)cc6)nc(-c6ccc7ccccc7c6)n5)c4c3c2)cc1 ; c1ccc(-c2cc(-c3nc(-c4ccc(-c5cccc6ccccc56)cc4)nc(-c4ccc5ccccc5c4)n3)c3c(c2)oc2ccccc23)cc1 ; c1ccc(-c2ccc3c(c2)oc2cccc(-c4nc(-c5ccc(-c6cccc7ccccc67)cc5)nc(-c5ccc6ccccc6c5)n4)c23)cc1]

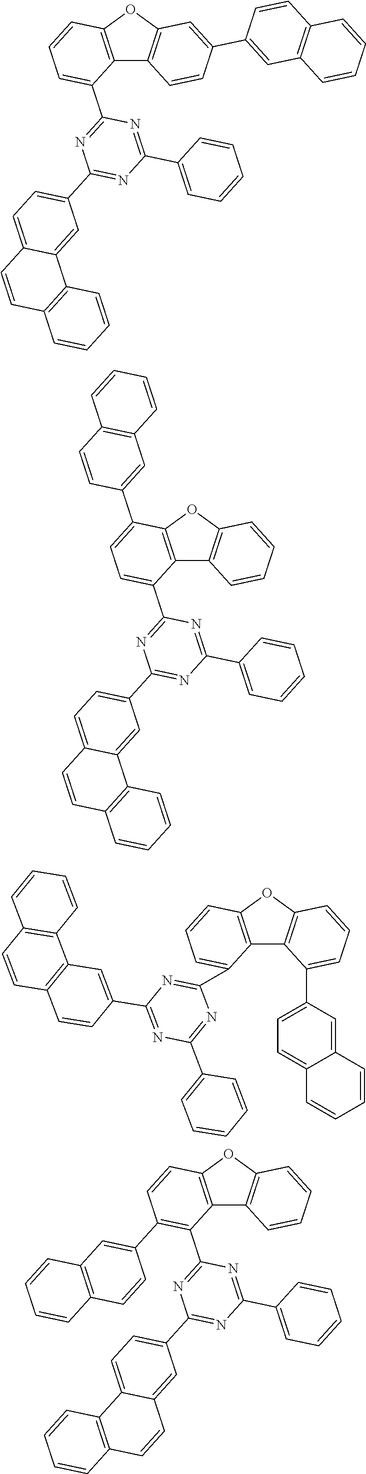

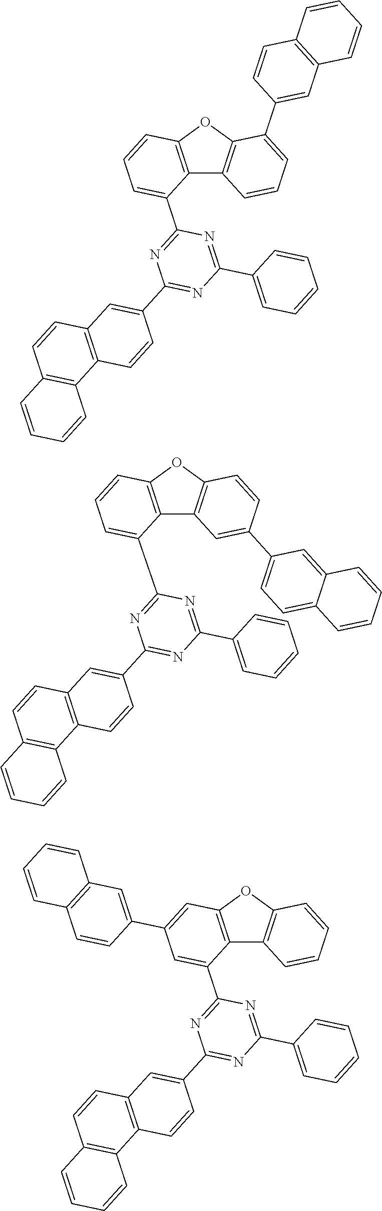

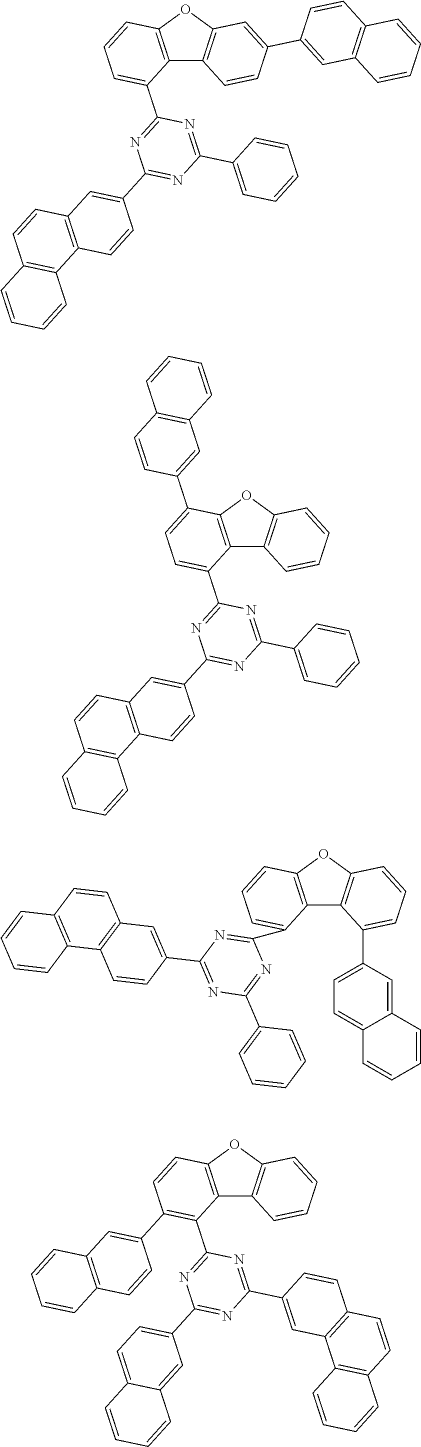 [SMILES: c1ccc(-c2nc(-c3ccc4c(ccc5ccccc54)c3)nc(-c3cccc4oc5cc(-c6ccc7ccccc7c6)ccc5c34)n2)cc1 ; c1ccc(-c2nc(-c3ccc4c(ccc5ccccc54)c3)nc(-c3ccc(-c4ccc5ccccc5c4)c4oc5ccccc5c34)n2)cc1 ; c1ccc(-c2nc(-c3ccc4c(ccc5ccccc54)c3)nc(-c3cccc4oc5cccc(-c6ccc7ccccc7c6)c5c34)n2)cc1 ; c1ccc2cc(-c3nc(-c4ccc5ccc6ccccc6c5c4)nc(-c4c(-c5ccc6ccccc6c5)ccc5oc6ccccc6c45)n3)ccc2c1]

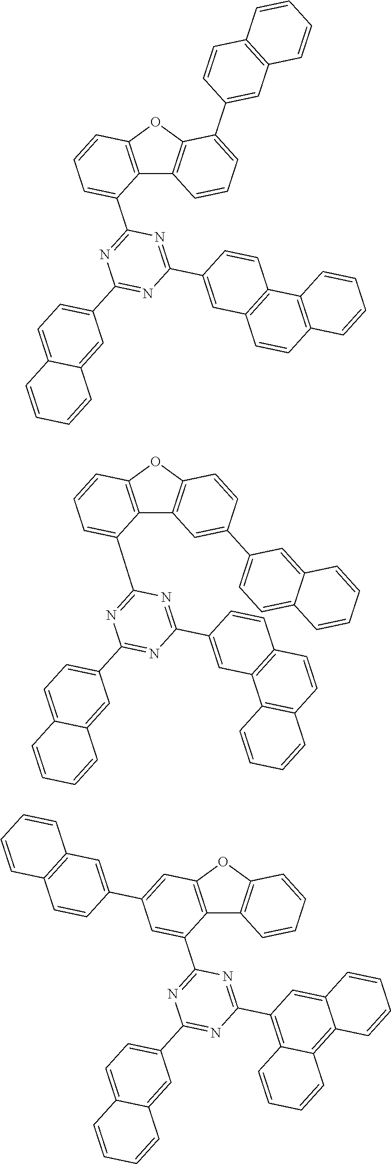

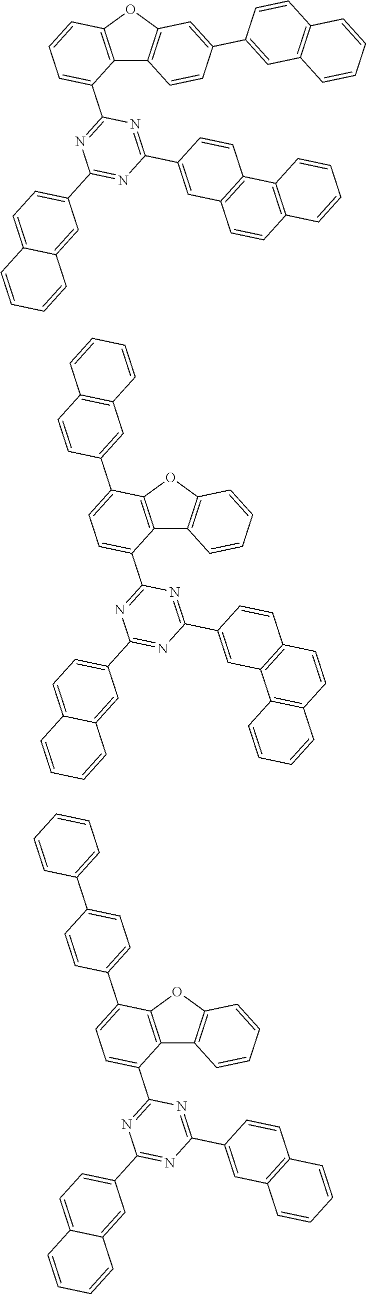

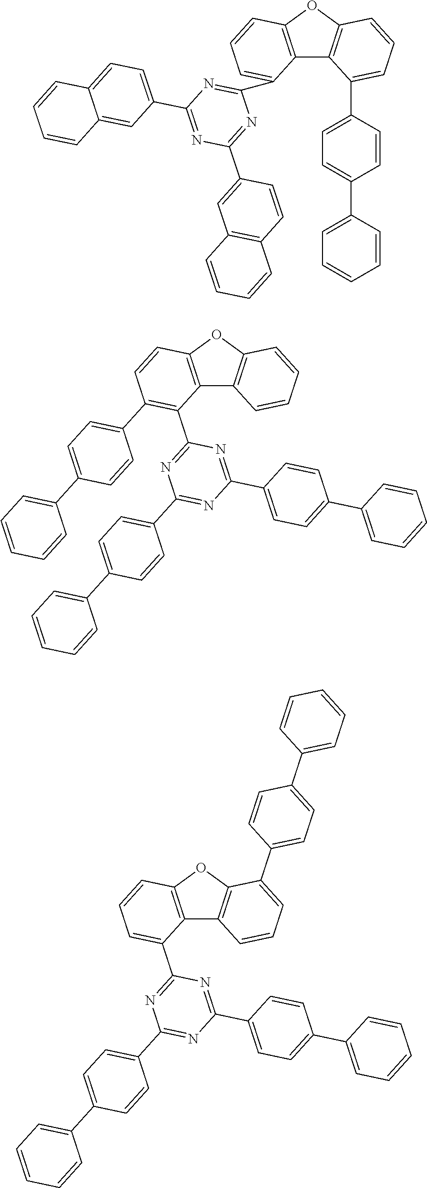

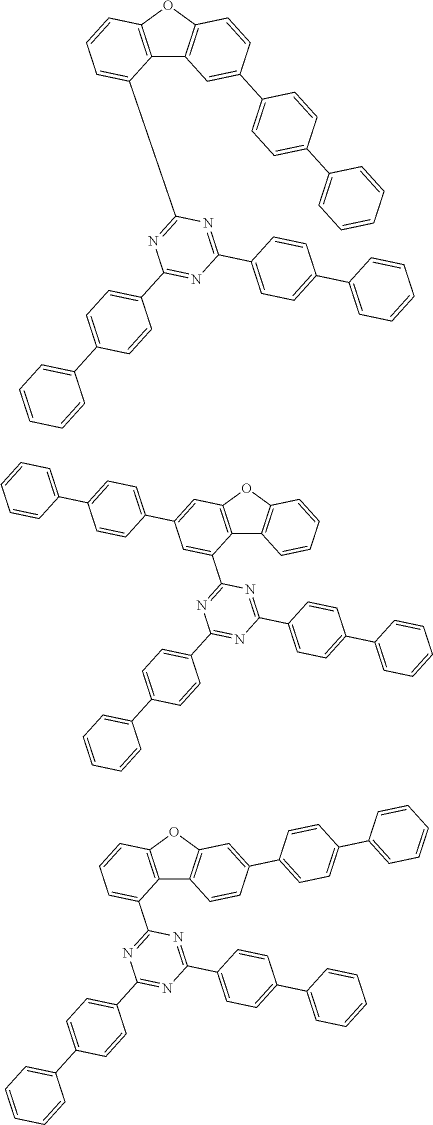

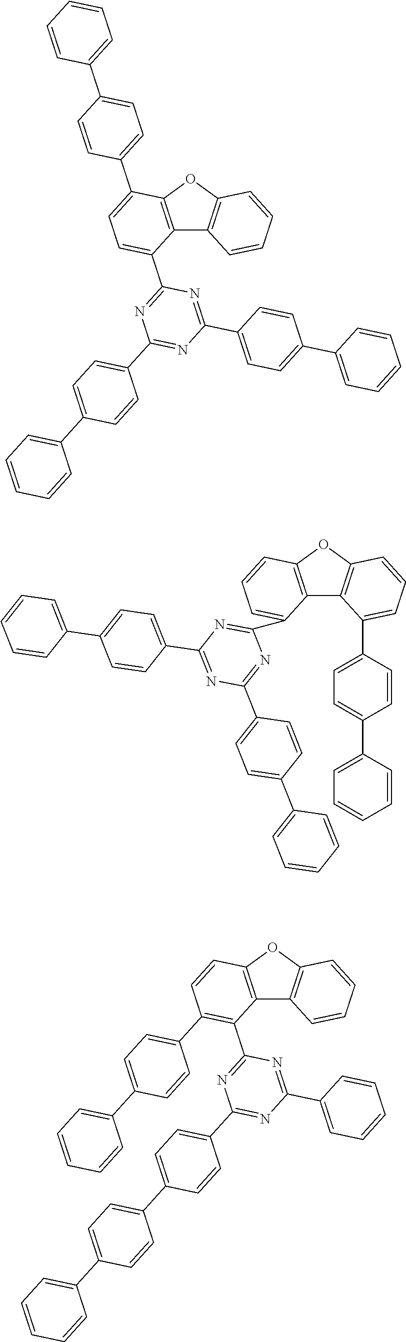 [SMILES: c1ccc(-c2ccc(-c3nc(-c4ccc(-c5ccccc5)cc4)nc(-c4ccc(-c5ccc(-c6ccccc6)cc5)c5oc6ccccc6c45)n3)cc2)cc1 ; c1ccc(-c2ccc(-c3nc(-c4ccc(-c5ccccc5)cc4)nc(-c4cccc5oc6cccc(-c7ccc(-c8ccccc8)cc7)c6c45)n3)cc2)cc1 ; c1ccc(-c2ccc(-c3ccc(-c4nc(-c5ccccc5)nc(-c5c(-c6ccc(-c7ccccc7)cc6)ccc6oc7ccccc7c56)n4)cc3)cc2)cc1]

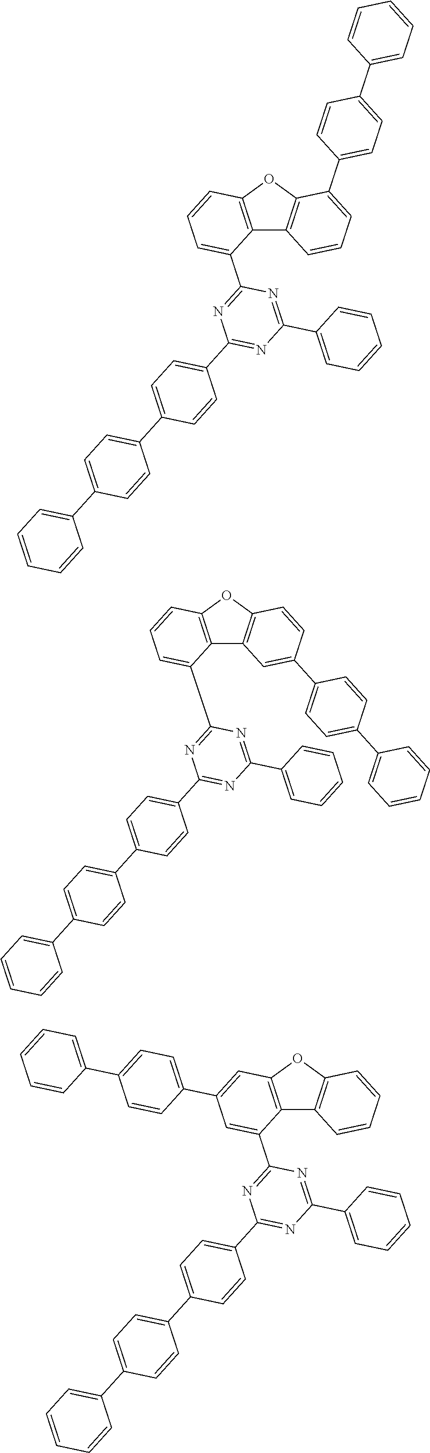

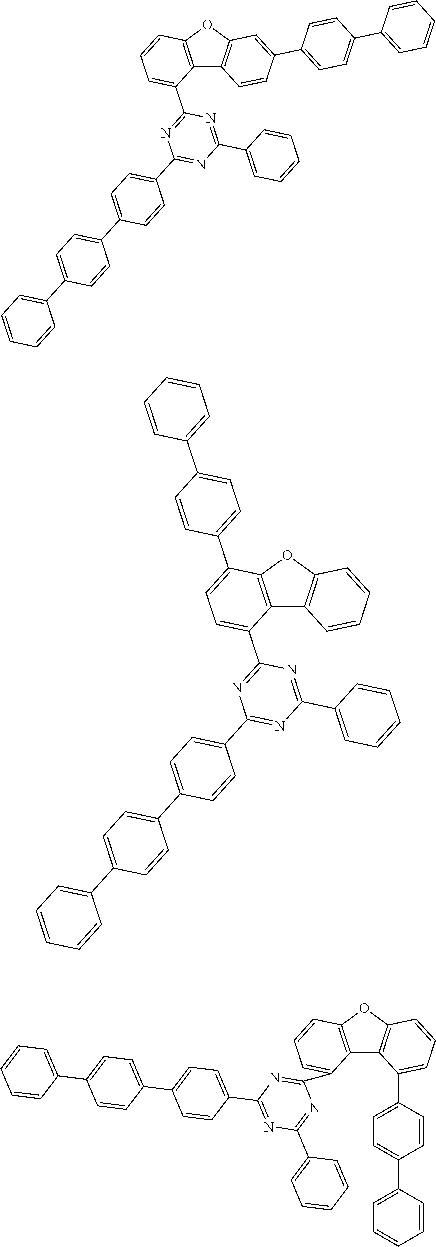 [SMILES: c1ccc(-c2ccc(-c3ccc(-c4nc(-c5ccccc5)nc(-c5cccc6oc7cc(-c8ccc(-c9ccccc9)cc8)ccc7c56)n4)cc3)cc2)cc1 ; c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1]

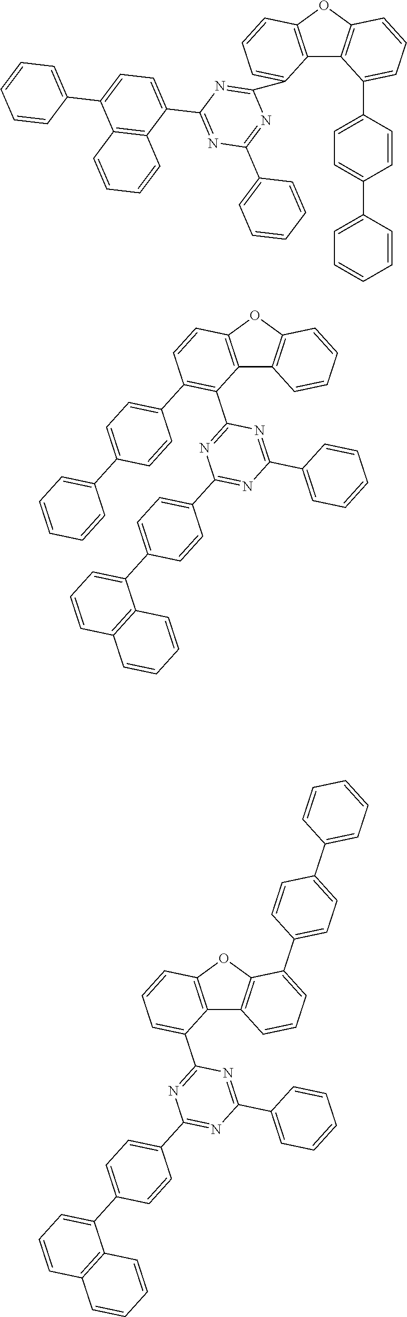 [SMILES: c1ccc(-c2ccc(-c3cccc4oc5cccc(-c6nc(-c7ccccc7)nc(-c7ccc(-c8ccccc8)c8ccccc78)n6)c5c34)cc2)cc1 ; c1ccc(-c2ccc(-c3ccc4oc5ccccc5c4c3-c3nc(-c4ccccc4)nc(-c4ccc(-c5cccc6ccccc56)cc4)n3)cc2)cc1 ; c1ccc(-c2ccc(-c3cccc4c3oc3cccc(-c5nc(-c6ccccc6)nc(-c6ccc(-c7cccc8ccccc78)cc6)n5)c34)cc2)cc1]

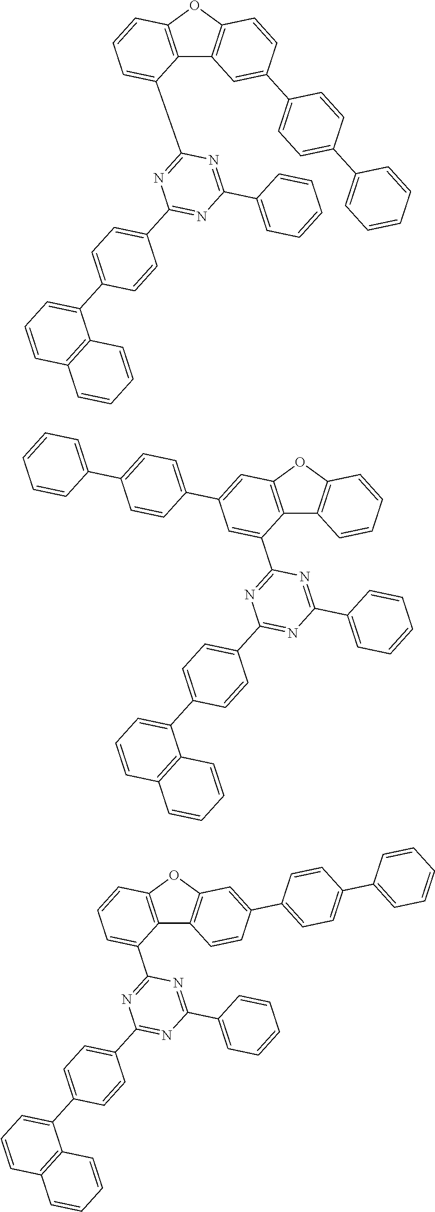

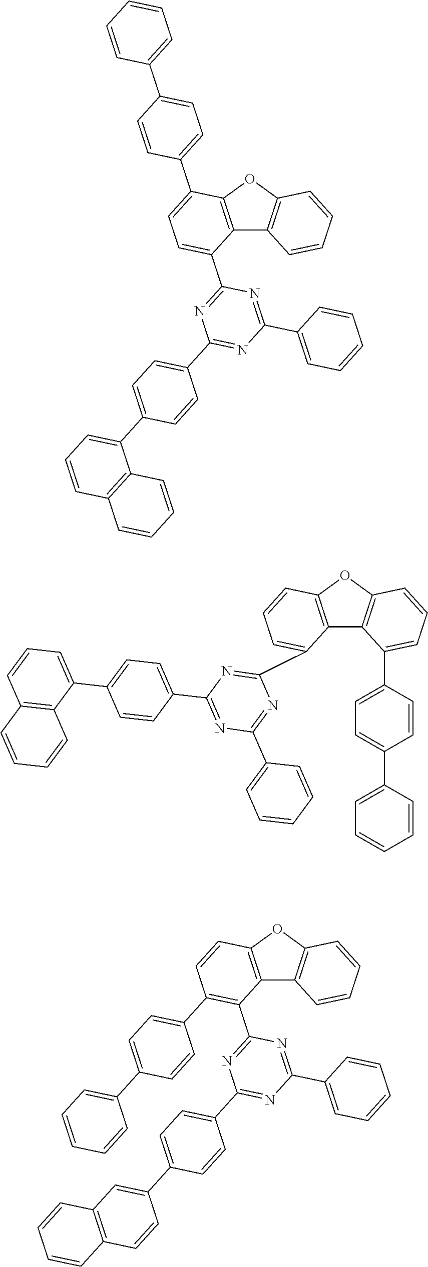 [SMILES: c1ccc(-c2ccc(-c3ccc(-c4nc(-c5ccccc5)nc(-c5ccc(-c6cccc7ccccc67)cc5)n4)c4c3oc3ccccc34)cc2)cc1 ; c1ccc(-c2ccc(-c3cccc4oc5cccc(-c6nc(-c7ccccc7)nc(-c7ccc(-c8cccc9ccccc89)cc7)n6)c5c34)cc2)cc1 ; c1ccc(-c2ccc(-c3ccc4oc5ccccc5c4c3-c3nc(-c4ccccc4)nc(-c4ccc(-c5ccc6ccccc6c5)cc4)n3)cc2)cc1]

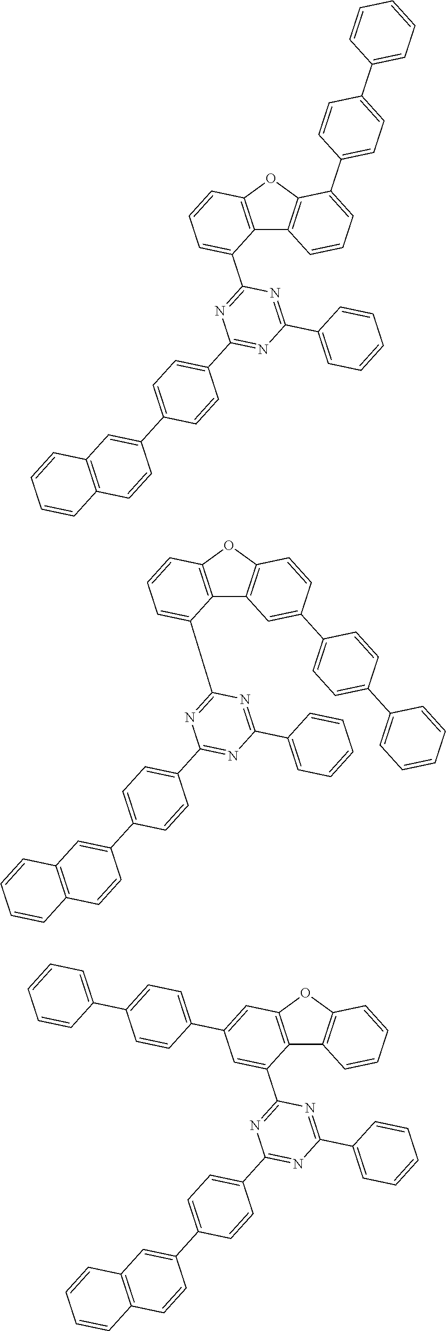

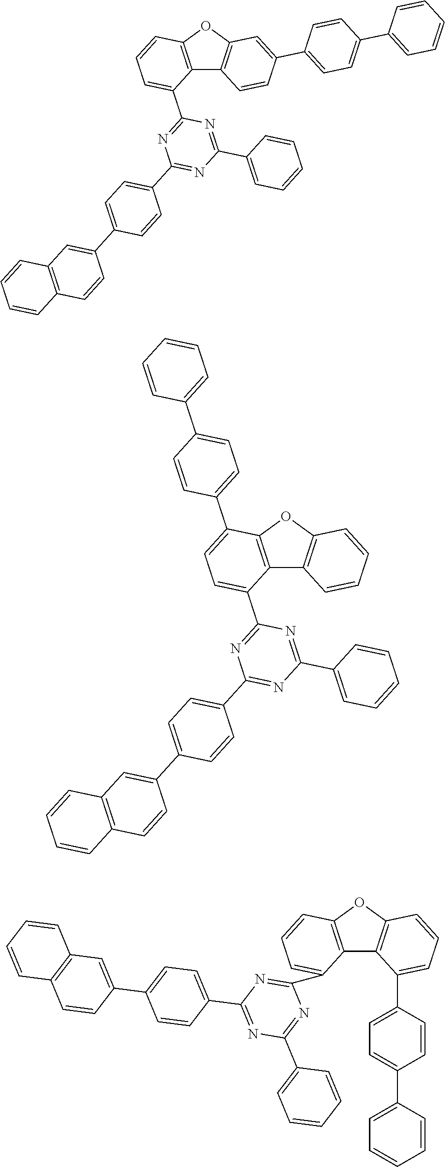 [SMILES: c1ccc(-c2ccc(-c3ccc4c(c3)oc3cccc(-c5nc(-c6ccccc6)nc(-c6ccc(-c7ccc8ccccc8c7)cc6)n5)c34)cc2)cc1 ; c1ccc(-c2ccc(-c3ccc(-c4nc(-c5ccccc5)nc(-c5ccc(-c6ccc7ccccc7c6)cc5)n4)c4c3oc3ccccc34)cc2)cc1 ; c1ccc(-c2ccc(-c3cccc4oc5cccc(-c6nc(-c7ccccc7)nc(-c7ccc(-c8ccc9ccccc9c8)cc7)n6)c5c34)cc2)cc1]

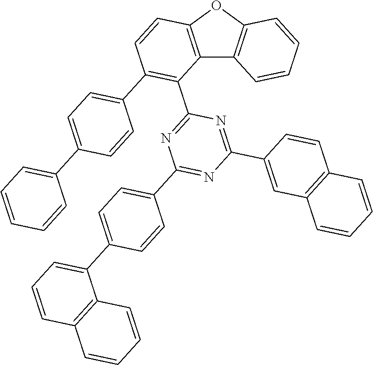 [SMILES: c1ccc(-c2ccc(-c3ccc4oc5ccccc5c4c3-c3nc(-c4ccc(-c5cccc6ccccc56)cc4)nc(-c4ccc5ccccc5c4)n3)cc2)cc1]

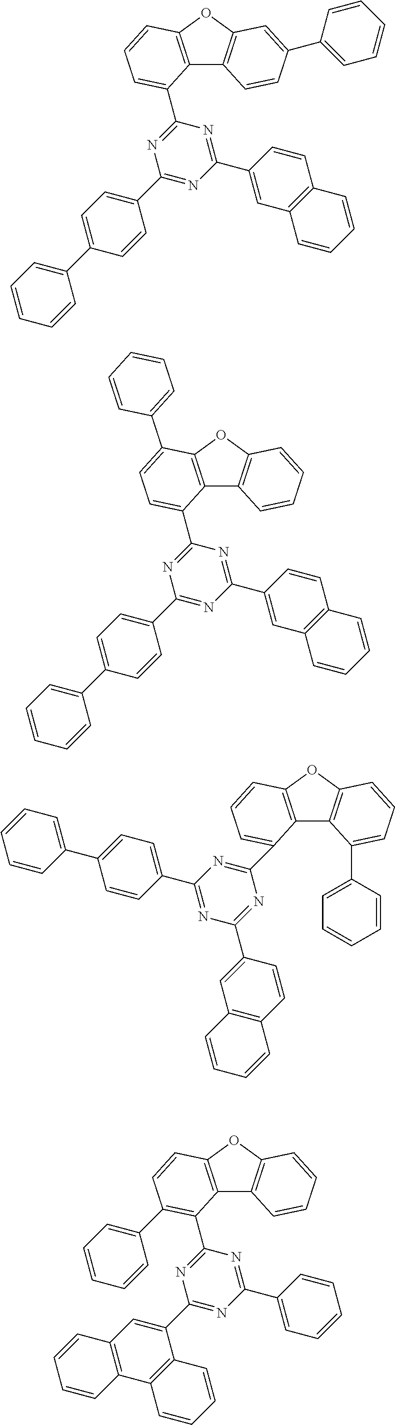 [SMILES: c1ccc(-c2ccc(-c3nc(-c4ccc5ccccc5c4)nc(-c4cccc5oc6cc(-c7ccccc7)ccc6c45)n3)cc2)cc1 ; c1ccc(-c2ccc(-c3nc(-c4ccc5ccccc5c4)nc(-c4ccc(-c5ccccc5)c5oc6ccccc6c45)n3)cc2)cc1 ; c1ccc(-c2ccc(-c3nc(-c4ccc5ccccc5c4)nc(-c4cccc5oc6cccc(-c7ccccc7)c6c45)n3)cc2)cc1 ; c1ccc(-c2nc(-c3cc4ccccc4c4ccccc34)nc(-c3c(-c4ccccc4)ccc4oc5ccccc5c34)n2)cc1]

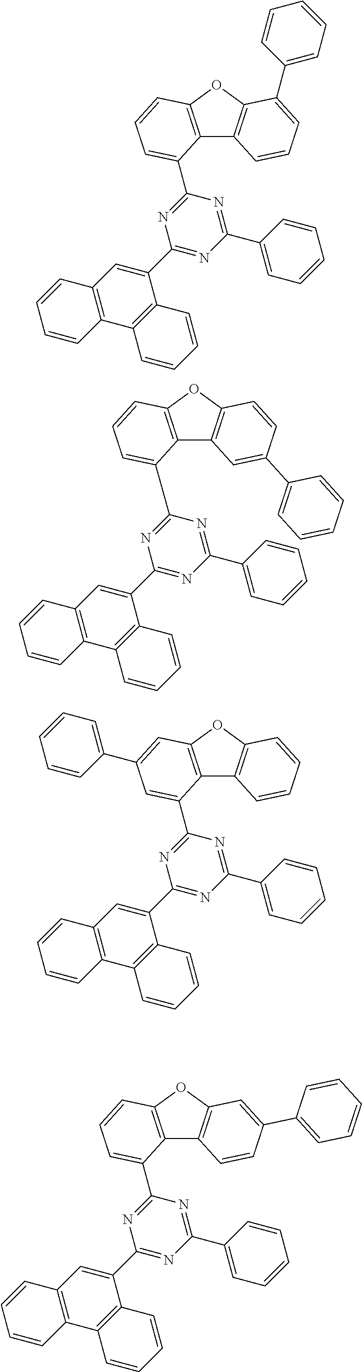

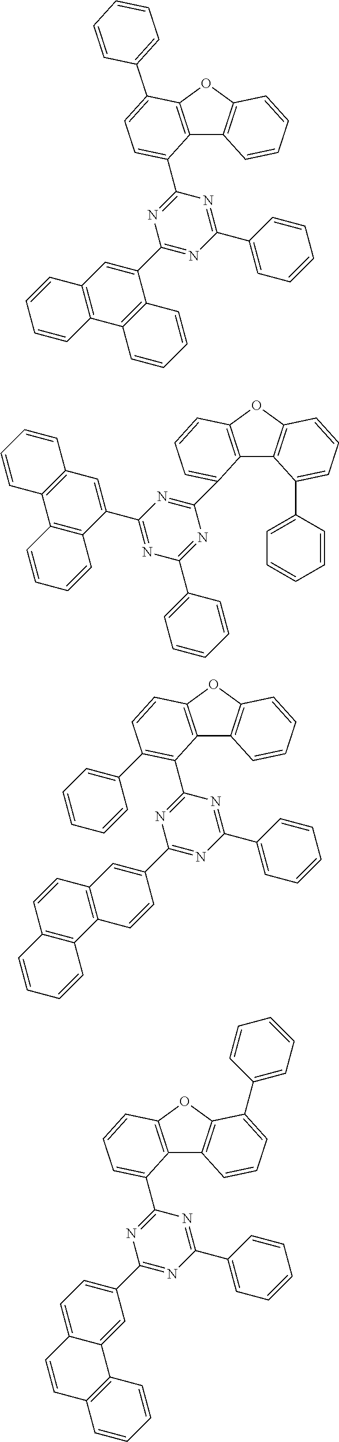

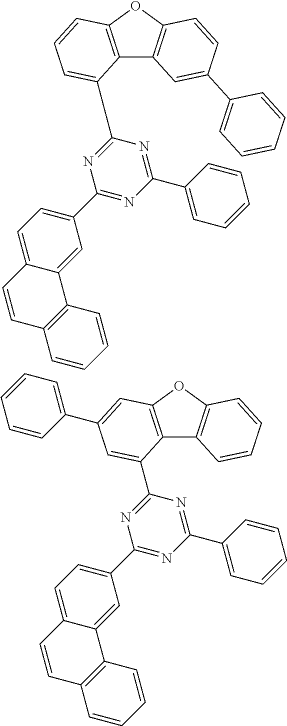

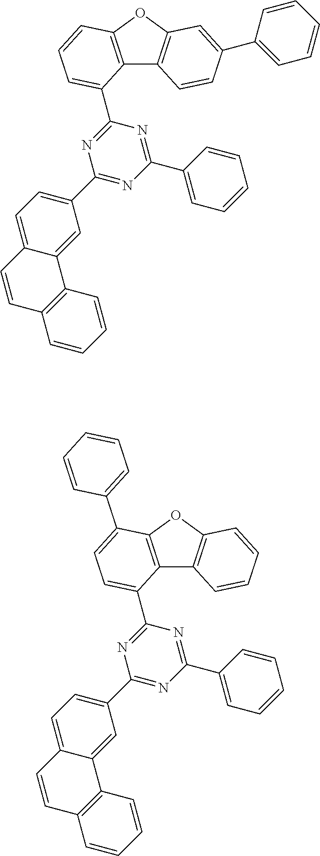 [SMILES: c1ccc(-c2ccc3c(c2)oc2cccc(-c4nc(-c5ccccc5)nc(-c5ccc6ccc7ccccc7c6c5)n4)c23)cc1 ; c1ccc(-c2nc(-c3ccc4ccc5ccccc5c4c3)nc(-c3ccc(-c4ccccc4)c4oc5ccccc5c34)n2)cc1]

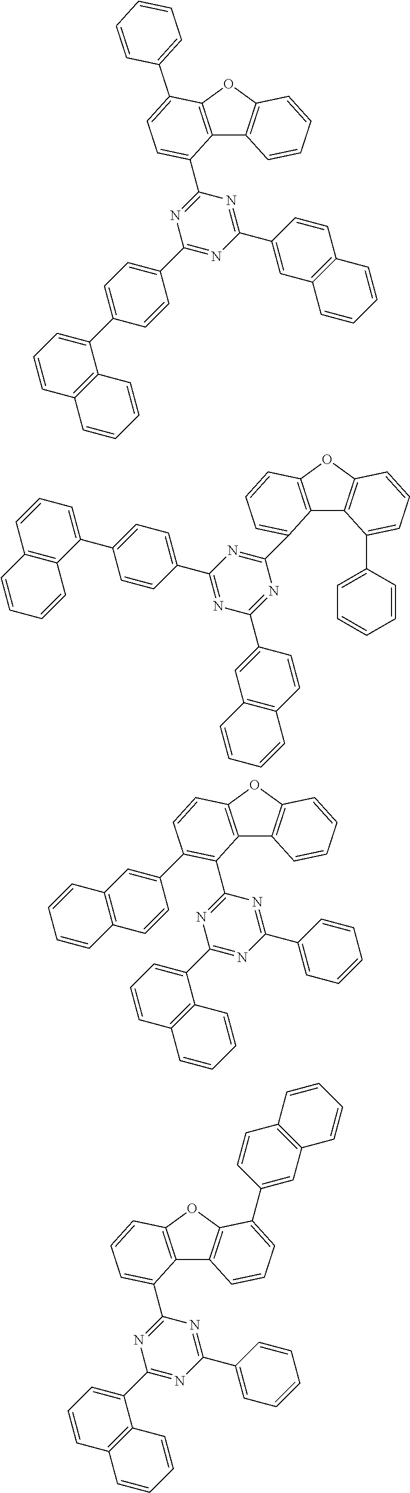

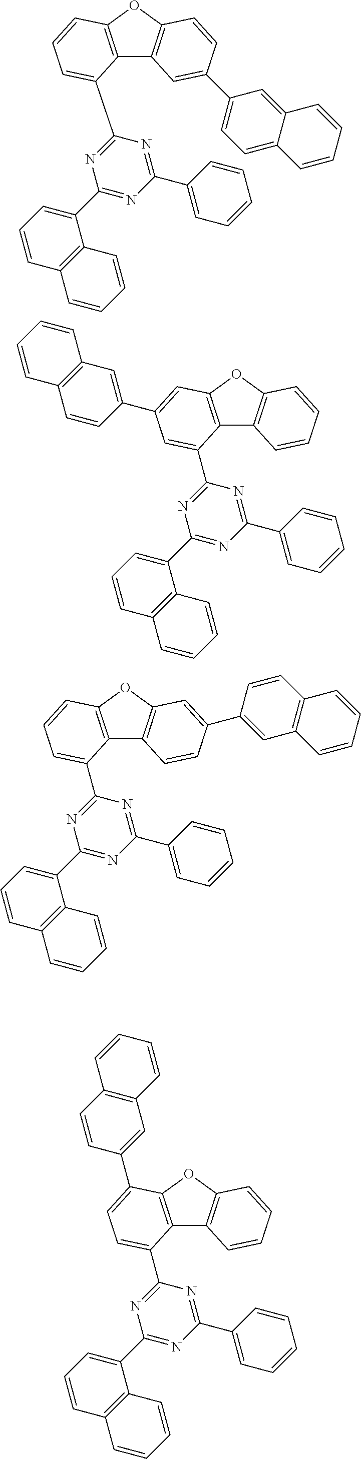

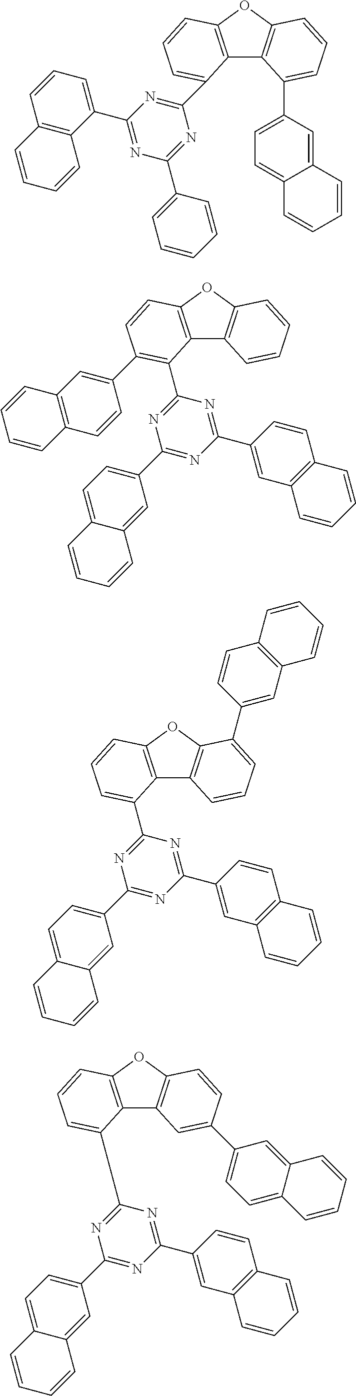

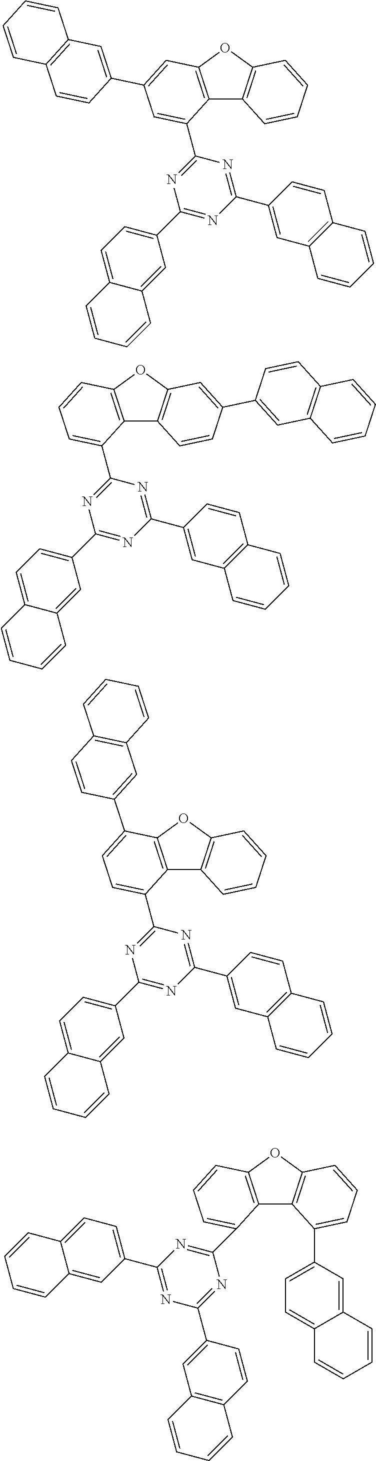

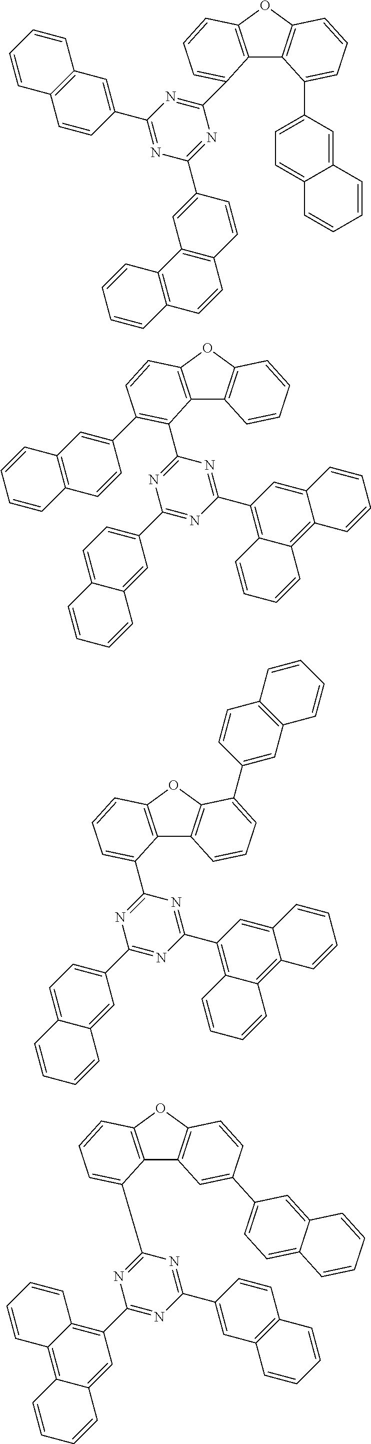

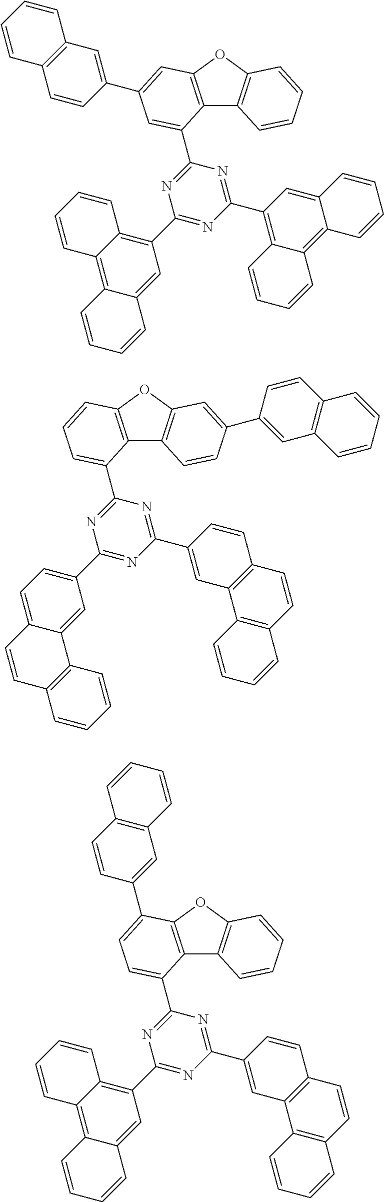

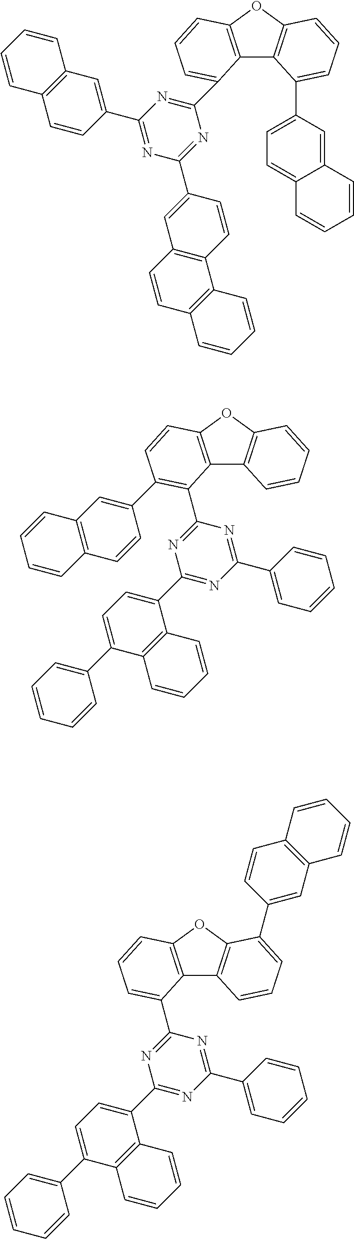 [SMILES: c1ccc2cc(-c3nc(-c4ccc5c(ccc6ccccc65)c4)nc(-c4cccc5oc6cccc(-c7ccc8ccccc8c7)c6c45)n3)ccc2c1 ; c1ccc(-c2nc(-c3ccc(-c4ccccc4)c4ccccc34)nc(-c3c(-c4ccc5ccccc5c4)ccc4oc5ccccc5c34)n2)cc1 ; c1ccc(-c2nc(-c3ccc(-c4ccccc4)c4ccccc34)nc(-c3cccc4oc5c(-c6ccc7ccccc7c6)cccc5c34)n2)cc1]

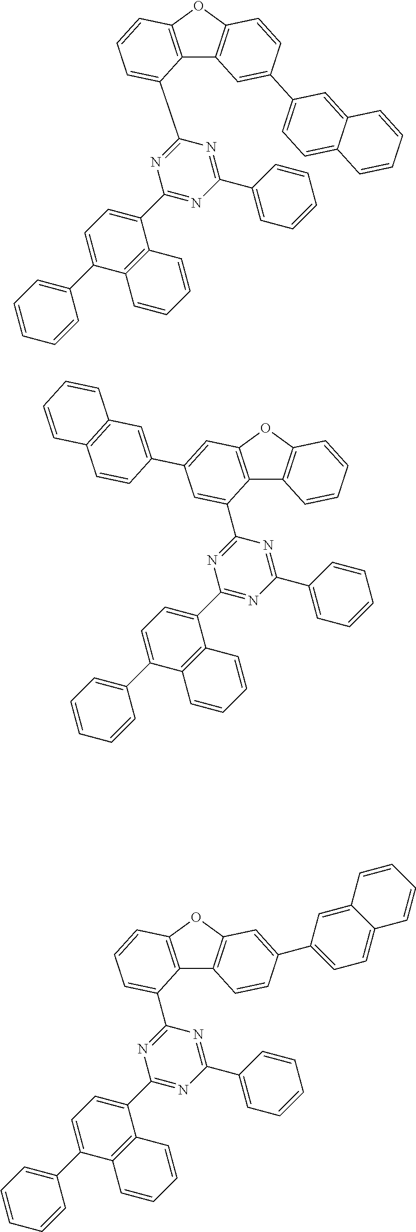

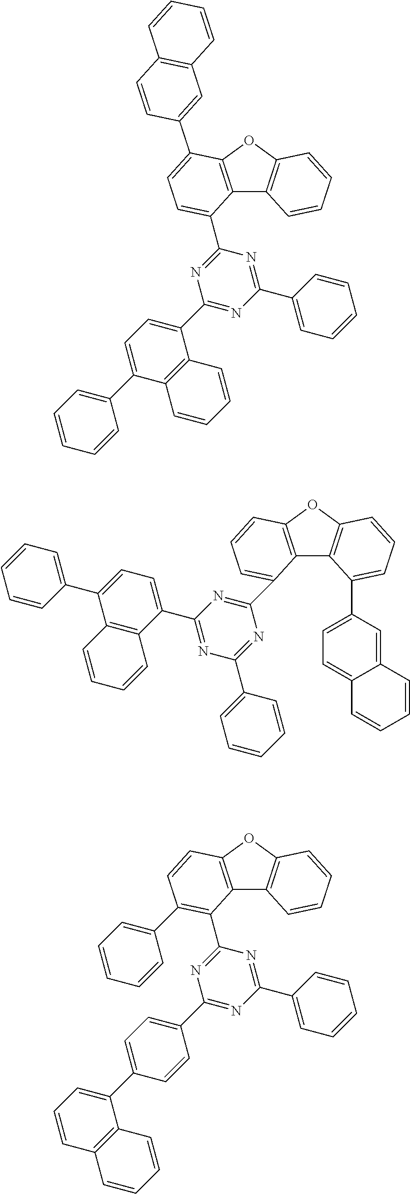 [SMILES: c1ccc(-c2nc(-c3ccc(-c4ccccc4)c4ccccc34)nc(-c3ccc(-c4ccc5ccccc5c4)c4oc5ccccc5c34)n2)cc1 ; c1ccc(-c2nc(-c3ccc(-c4ccccc4)c4ccccc34)nc(-c3cccc4oc5cccc(-c6ccc7ccccc7c6)c5c34)n2)cc1 ; c1ccc(-c2nc(-c3ccc(-c4cccc5ccccc45)cc3)nc(-c3c(-c4ccccc4)ccc4oc5ccccc5c34)n2)cc1]

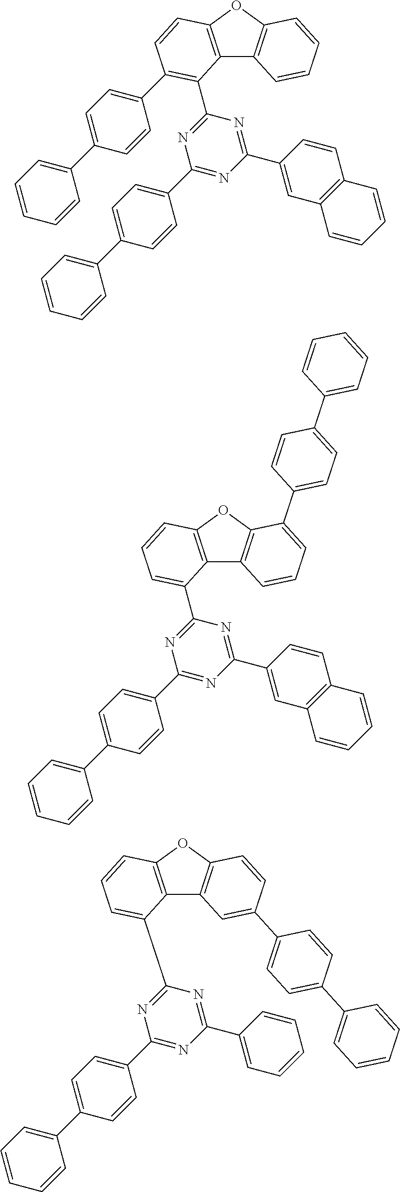 [SMILES: c1ccc(-c2ccc(-c3nc(-c4ccc5ccccc5c4)nc(-c4c(-c5ccc(-c6ccccc6)cc5)ccc5oc6ccccc6c45)n3)cc2)cc1 ; c1ccc(-c2ccc(-c3nc(-c4ccc5ccccc5c4)nc(-c4cccc5oc6c(-c7ccc(-c8ccccc8)cc7)cccc6c45)n3)cc2)cc1 ; c1ccc(-c2ccc(-c3ccc4oc5cccc(-c6nc(-c7ccccc7)nc(-c7ccc(-c8ccccc8)cc7)n6)c5c4c3)cc2)cc1]

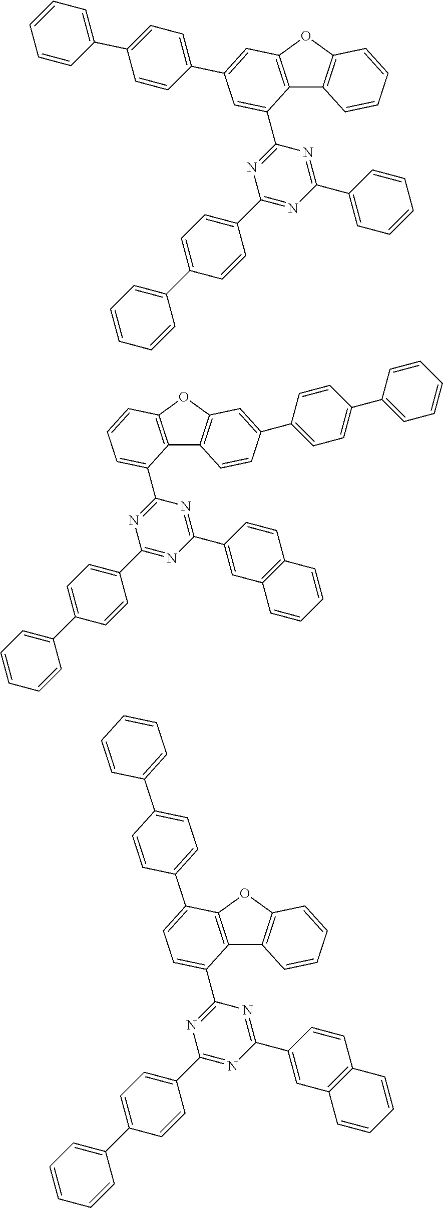 [SMILES: c1ccc(-c2ccc(-c3cc(-c4nc(-c5ccccc5)nc(-c5ccc(-c6ccccc6)cc5)n4)c4c(c3)oc3ccccc34)cc2)cc1 ; c1ccc(-c2ccc(-c3ccc4c(c3)oc3cccc(-c5nc(-c6ccc(-c7ccccc7)cc6)nc(-c6ccc7ccccc7c6)n5)c34)cc2)cc1 ; c1ccc(-c2ccc(-c3nc(-c4ccc5ccccc5c4)nc(-c4ccc(-c5ccc(-c6ccccc6)cc5)c5oc6ccccc6c45)n3)cc2)cc1]

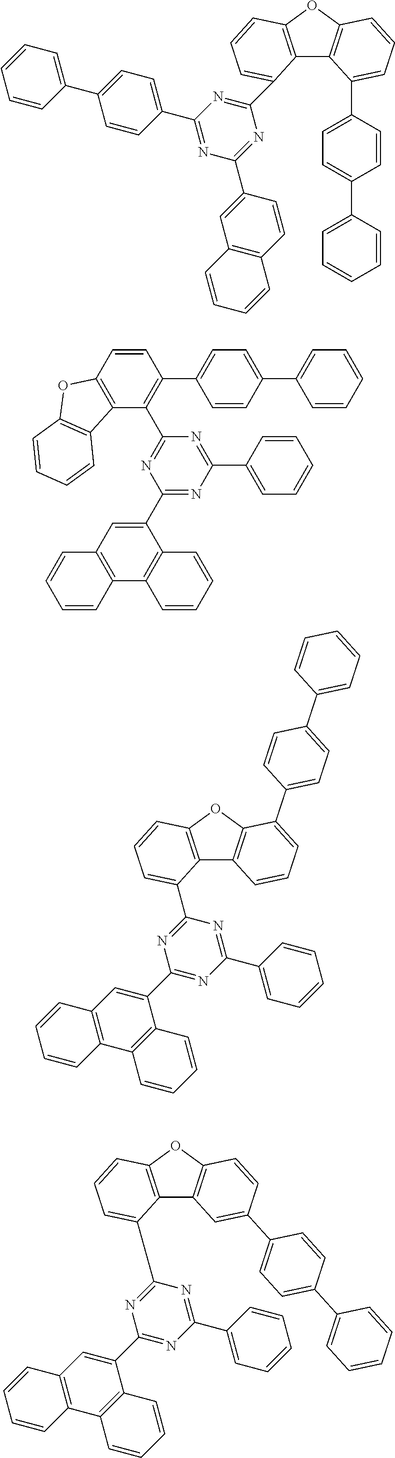

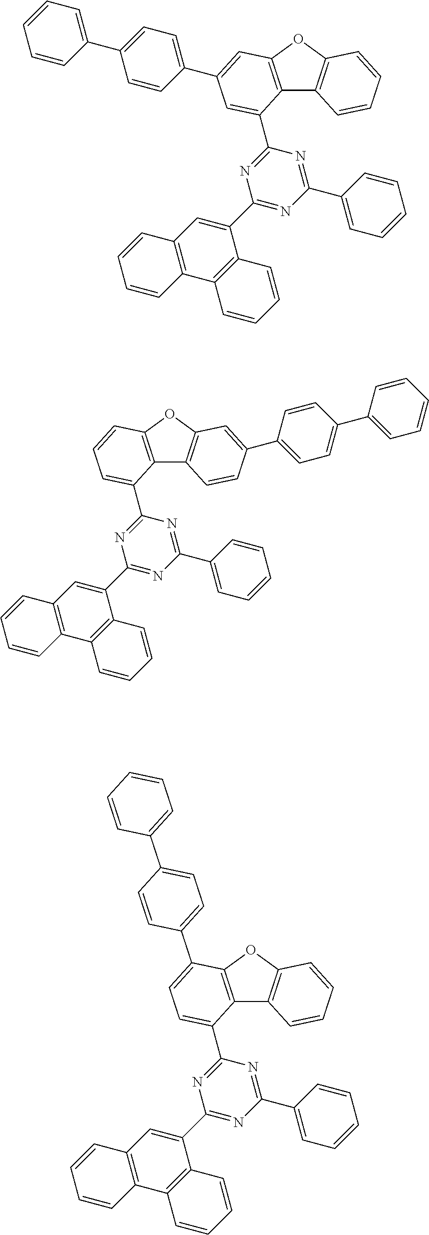 [SMILES: c1ccc(-c2ccc(-c3cc(-c4nc(-c5ccccc5)nc(-c5cc6ccccc6c6ccccc56)n4)c4c(c3)oc3ccccc34)cc2)cc1 ; c1ccc(-c2ccc(-c3ccc4c(c3)oc3cccc(-c5nc(-c6ccccc6)nc(-c6cc7ccccc7c7ccccc67)n5)c34)cc2)cc1 ; c1ccc(-c2ccc(-c3ccc(-c4nc(-c5ccccc5)nc(-c5cc6ccccc6c6ccccc56)n4)c4c3oc3ccccc34)cc2)cc1]

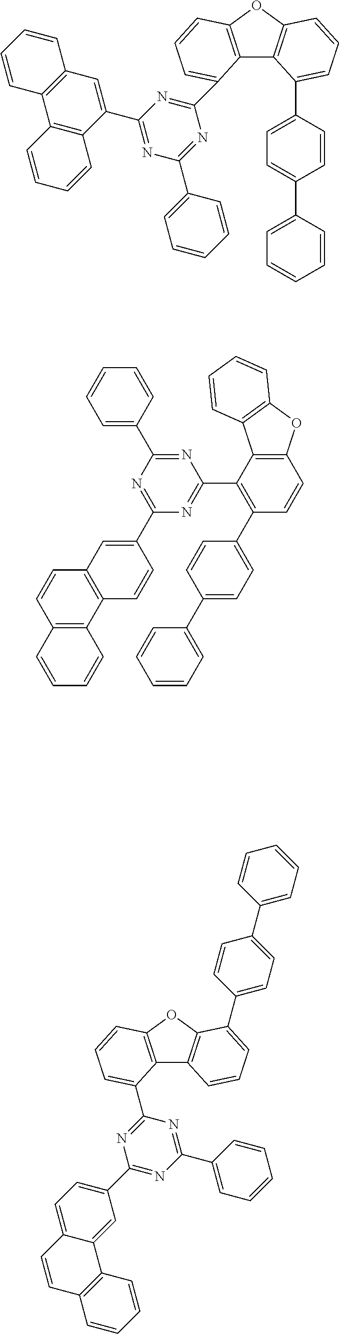 [SMILES: c1ccc(-c2ccc(-c3cccc4oc5cccc(-c6nc(-c7ccccc7)nc(-c7cc8ccccc8c8ccccc78)n6)c5c34)cc2)cc1 ; c1ccc(-c2ccc(-c3ccc4oc5ccccc5c4c3-c3nc(-c4ccccc4)nc(-c4ccc5c(ccc6ccccc65)c4)n3)cc2)cc1 ; c1ccc(-c2ccc(-c3cccc4c3oc3cccc(-c5nc(-c6ccccc6)nc(-c6ccc7ccc8ccccc8c7c6)n5)c34)cc2)cc1]

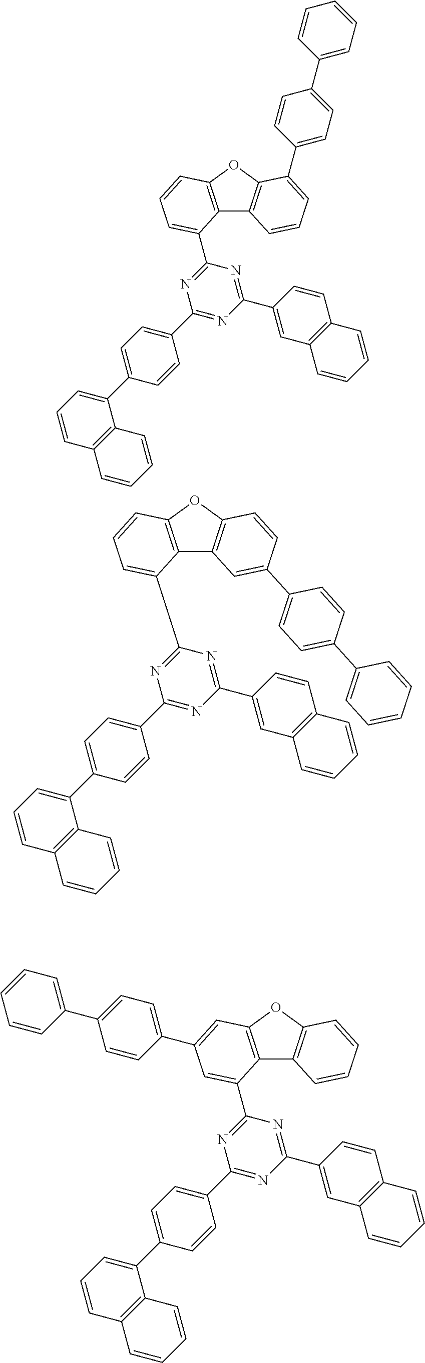

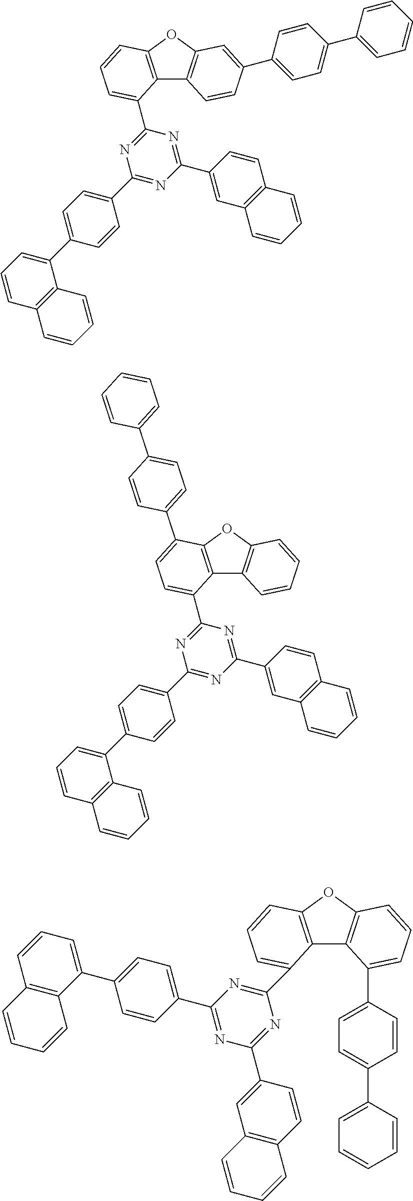 [SMILES: c1ccc(-c2ccc(-c3ccc4c(c3)oc3cccc(-c5nc(-c6ccc(-c7cccc8ccccc78)cc6)nc(-c6ccc7ccccc7c6)n5)c34)cc2)cc1 ; c1ccc(-c2ccc(-c3ccc(-c4nc(-c5ccc(-c6cccc7ccccc67)cc5)nc(-c5ccc6ccccc6c5)n4)c4c3oc3ccccc34)cc2)cc1 ; c1ccc(-c2ccc(-c3cccc4oc5cccc(-c6nc(-c7ccc(-c8cccc9ccccc89)cc7)nc(-c7ccc8ccccc8c7)n6)c5c34)cc2)cc1]

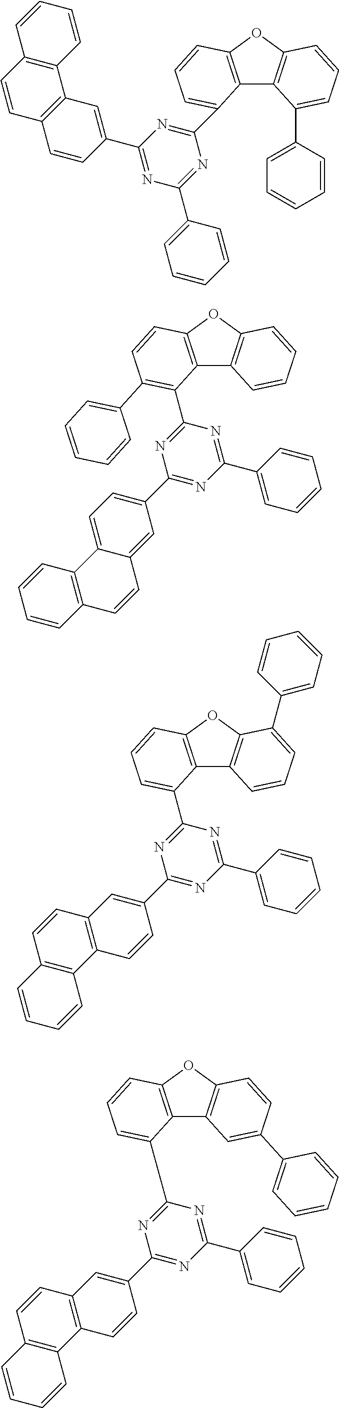

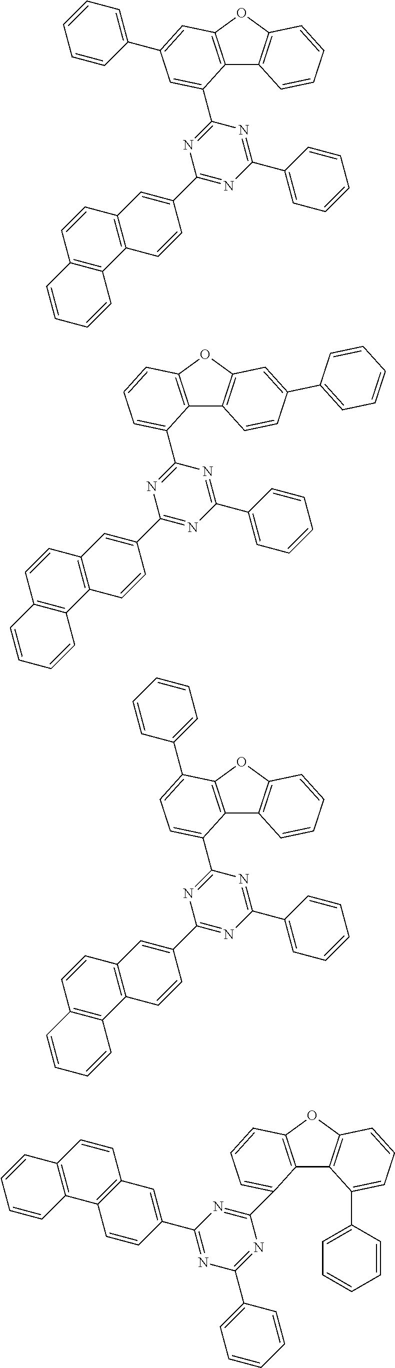 [SMILES: c1ccc(-c2cc(-c3nc(-c4ccccc4)nc(-c4ccc5c(ccc6ccccc65)c4)n3)c3c(c2)oc2ccccc23)cc1 ; c1ccc(-c2ccc3c(c2)oc2cccc(-c4nc(-c5ccccc5)nc(-c5ccc6c(ccc7ccccc76)c5)n4)c23)cc1 ; c1ccc(-c2nc(-c3ccc4c(ccc5ccccc54)c3)nc(-c3ccc(-c4ccccc4)c4oc5ccccc5c34)n2)cc1 ; c1ccc(-c2nc(-c3ccc4c(ccc5ccccc54)c3)nc(-c3cccc4oc5cccc(-c6ccccc6)c5c34)n2)cc1]

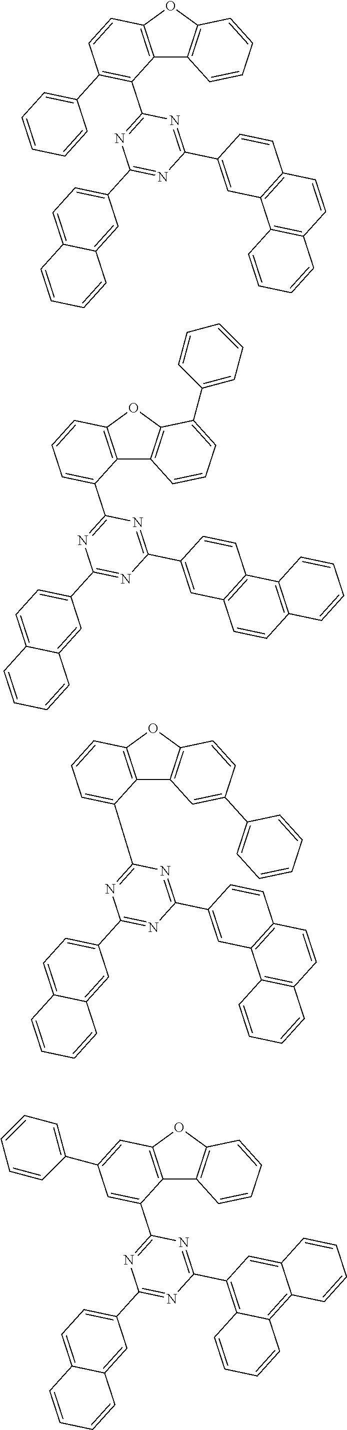 [SMILES: c1ccc(-c2ccc3oc4ccccc4c3c2-c2nc(-c3ccc4ccccc4c3)nc(-c3ccc4ccc5ccccc5c4c3)n2)cc1 ; c1ccc(-c2cccc3c2oc2cccc(-c4nc(-c5ccc6ccccc6c5)nc(-c5ccc6c(ccc7ccccc76)c5)n4)c23)cc1 ; c1ccc(-c2ccc3oc4cccc(-c5nc(-c6ccc7ccccc7c6)nc(-c6ccc7ccc8ccccc8c7c6)n5)c4c3c2)cc1 ; c1ccc(-c2cc(-c3nc(-c4ccc5ccccc5c4)nc(-c4cc5ccccc5c5ccccc45)n3)c3c(c2)oc2ccccc23)cc1]

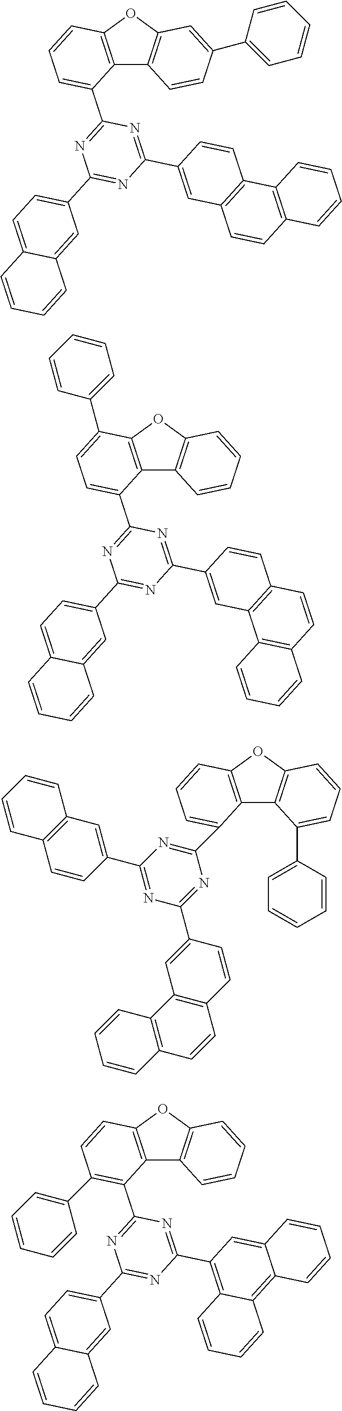

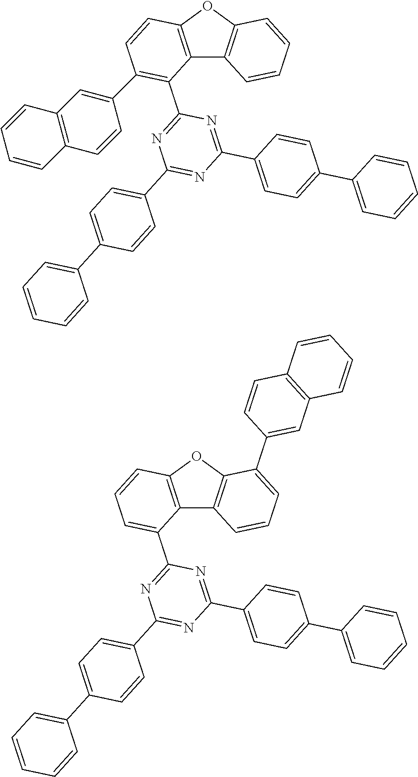 [SMILES: c1ccc(-c2ccc(-c3nc(-c4ccc(-c5ccccc5)cc4)nc(-c4c(-c5ccc6ccccc6c5)ccc5oc6ccccc6c45)n3)cc2)cc1 ; c1ccc(-c2ccc(-c3nc(-c4ccc(-c5ccccc5)cc4)nc(-c4cccc5oc6c(-c7ccc8ccccc8c7)cccc6c45)n3)cc2)cc1]

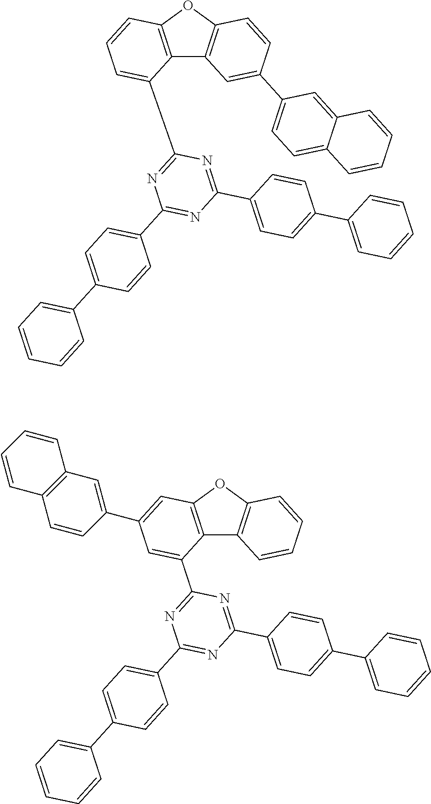 [SMILES: c1ccc(-c2ccc(-c3nc(-c4ccc(-c5ccccc5)cc4)nc(-c4cccc5oc6ccc(-c7ccc8ccccc8c7)cc6c45)n3)cc2)cc1 ; c1ccc(-c2ccc(-c3nc(-c4ccc(-c5ccccc5)cc4)nc(-c4cc(-c5ccc6ccccc6c5)cc5oc6ccccc6c45)n3)cc2)cc1]

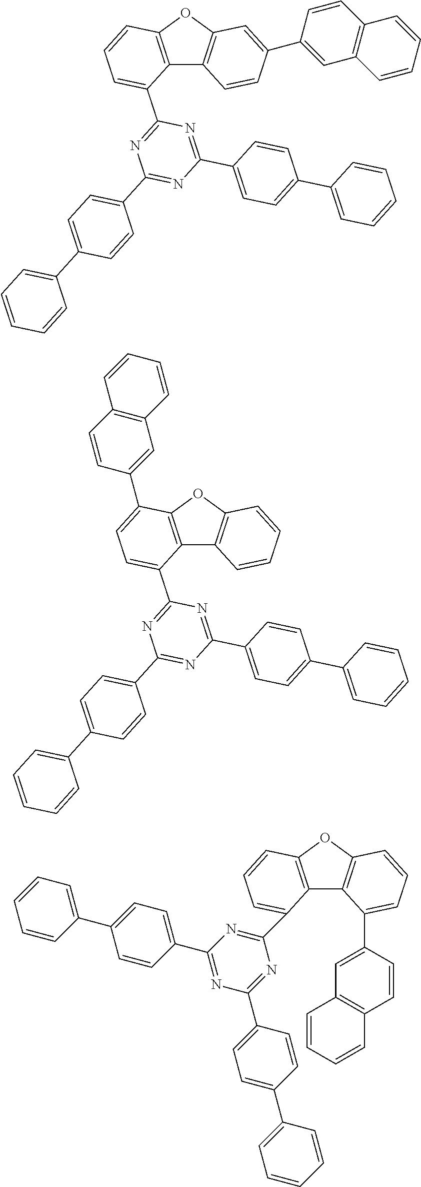 [SMILES: c1ccc(-c2ccc(-c3nc(-c4ccc(-c5ccccc5)cc4)nc(-c4cccc5oc6cc(-c7ccc8ccccc8c7)ccc6c45)n3)cc2)cc1 ; c1ccc(-c2ccc(-c3nc(-c4ccc(-c5ccccc5)cc4)nc(-c4ccc(-c5ccc6ccccc6c5)c5oc6ccccc6c45)n3)cc2)cc1 ; c1ccc(-c2ccc(-c3nc(-c4ccc(-c5ccccc5)cc4)nc(-c4cccc5oc6cccc(-c7ccc8ccccc8c7)c6c45)n3)cc2)cc1]

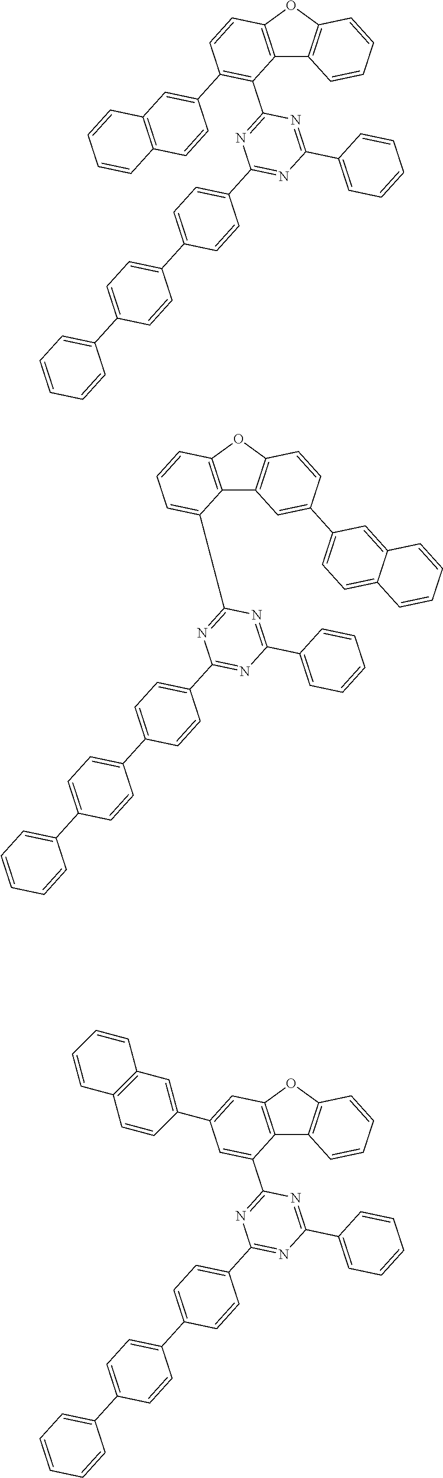 [SMILES: c1ccc(-c2ccc(-c3ccc(-c4nc(-c5ccccc5)nc(-c5c(-c6ccc7ccccc7c6)ccc6oc7ccccc7c56)n4)cc3)cc2)cc1 ; c1ccc(-c2ccc(-c3ccc(-c4nc(-c5ccccc5)nc(-c5cccc6oc7ccc(-c8ccc9ccccc9c8)cc7c56)n4)cc3)cc2)cc1 ; c1ccc(-c2ccc(-c3ccc(-c4nc(-c5ccccc5)nc(-c5cc(-c6ccc7ccccc7c6)cc6oc7ccccc7c56)n4)cc3)cc2)cc1]

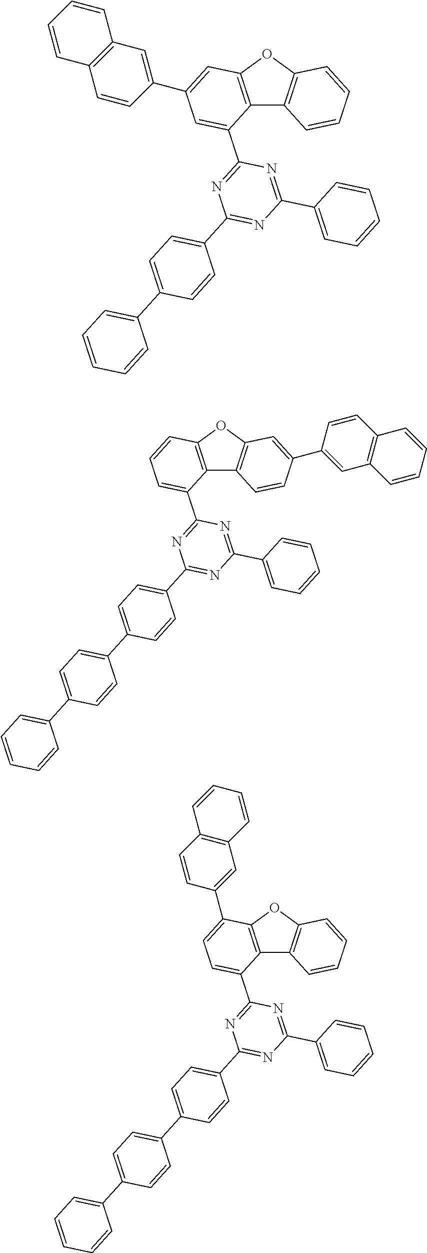 [SMILES: c1ccc(-c2ccc(-c3nc(-c4ccccc4)nc(-c4cc(-c5ccc6ccccc6c5)cc5oc6ccccc6c45)n3)cc2)cc1 ; c1ccc(-c2ccc(-c3ccc(-c4nc(-c5ccccc5)nc(-c5cccc6oc7cc(-c8ccc9ccccc9c8)ccc7c56)n4)cc3)cc2)cc1 ; c1ccc(-c2ccc(-c3ccc(-c4nc(-c5ccccc5)nc(-c5ccc(-c6ccc7ccccc7c6)c6oc7ccccc7c56)n4)cc3)cc2)cc1]

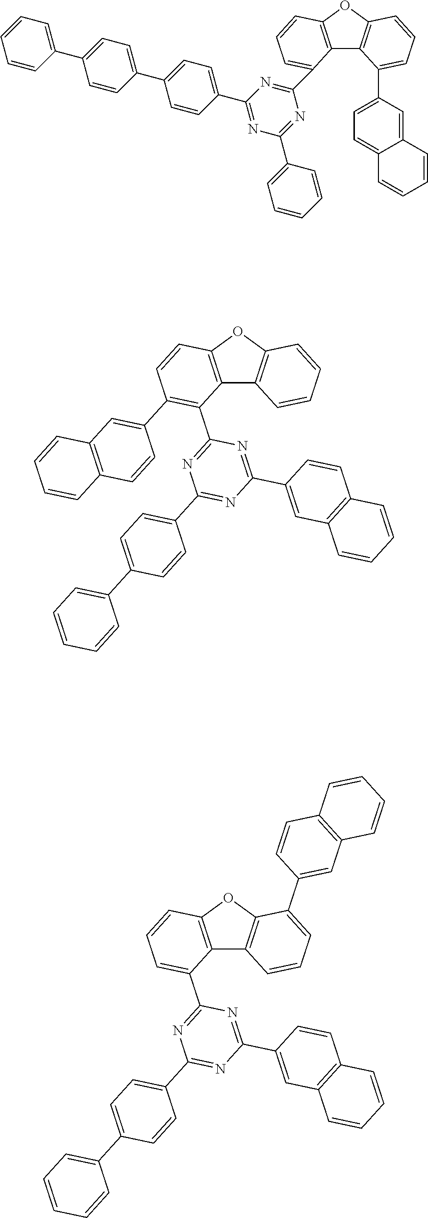 [SMILES: c1ccc(-c2ccc(-c3ccc(-c4nc(-c5ccccc5)nc(-c5cccc6oc7cccc(-c8ccc9ccccc9c8)c7c56)n4)cc3)cc2)cc1 ; c1ccc(-c2ccc(-c3nc(-c4ccc5ccccc5c4)nc(-c4c(-c5ccc6ccccc6c5)ccc5oc6ccccc6c45)n3)cc2)cc1 ; c1ccc(-c2ccc(-c3nc(-c4ccc5ccccc5c4)nc(-c4cccc5oc6c(-c7ccc8ccccc8c7)cccc6c45)n3)cc2)cc1]

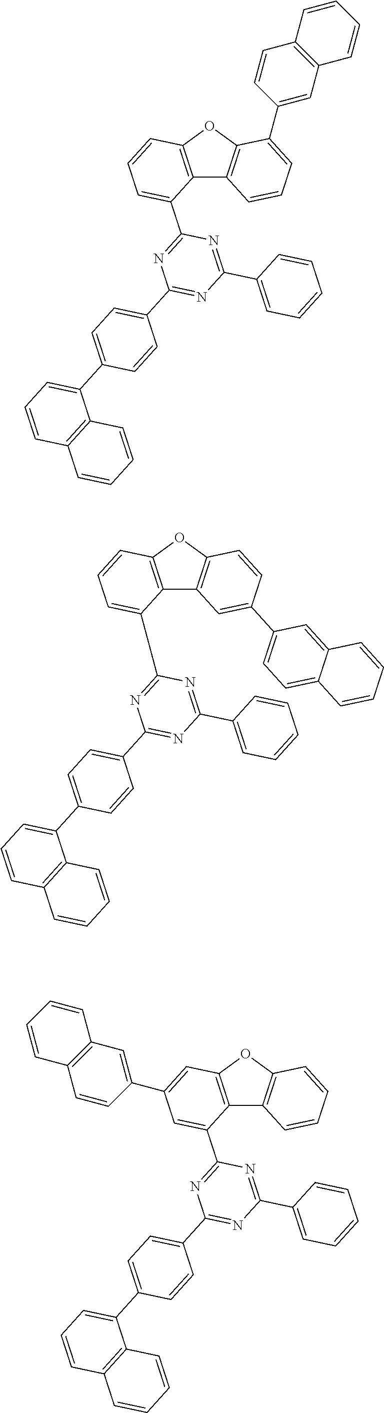 [SMILES: c1ccc(-c2nc(-c3ccc(-c4cccc5ccccc45)cc3)nc(-c3cccc4oc5c(-c6ccc7ccccc7c6)cccc5c34)n2)cc1 ; c1ccc(-c2nc(-c3ccc(-c4cccc5ccccc45)cc3)nc(-c3cccc4oc5ccc(-c6ccc7ccccc7c6)cc5c34)n2)cc1 ; c1ccc(-c2nc(-c3ccc(-c4cccc5ccccc45)cc3)nc(-c3cc(-c4ccc5ccccc5c4)cc4oc5ccccc5c34)n2)cc1]

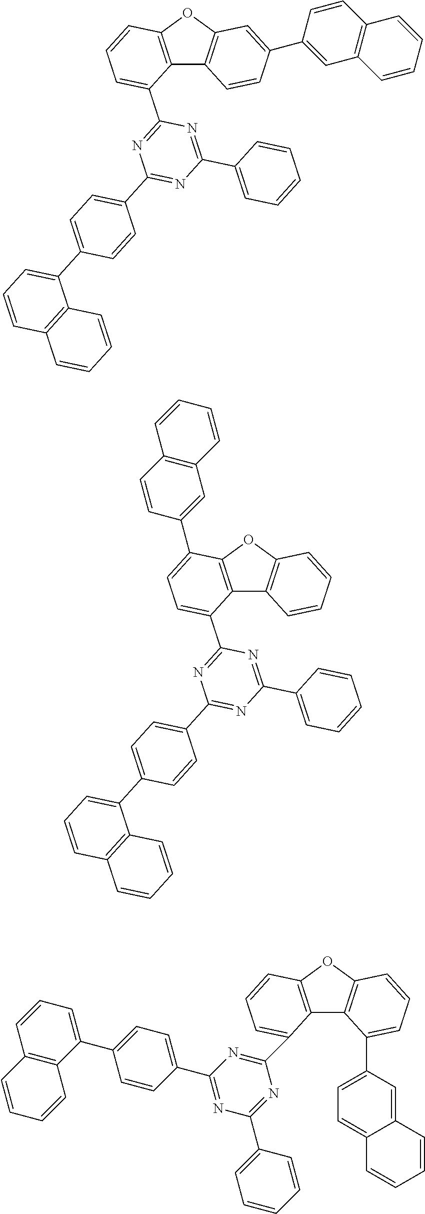 [SMILES: c1ccc(-c2nc(-c3ccc(-c4cccc5ccccc45)cc3)nc(-c3cccc4oc5cc(-c6ccc7ccccc7c6)ccc5c34)n2)cc1 ; c1ccc(-c2nc(-c3ccc(-c4cccc5ccccc45)cc3)nc(-c3ccc(-c4ccc5ccccc5c4)c4oc5ccccc5c34)n2)cc1 ; c1ccc(-c2nc(-c3ccc(-c4cccc5ccccc45)cc3)nc(-c3cccc4oc5cccc(-c6ccc7ccccc7c6)c5c34)n2)cc1]

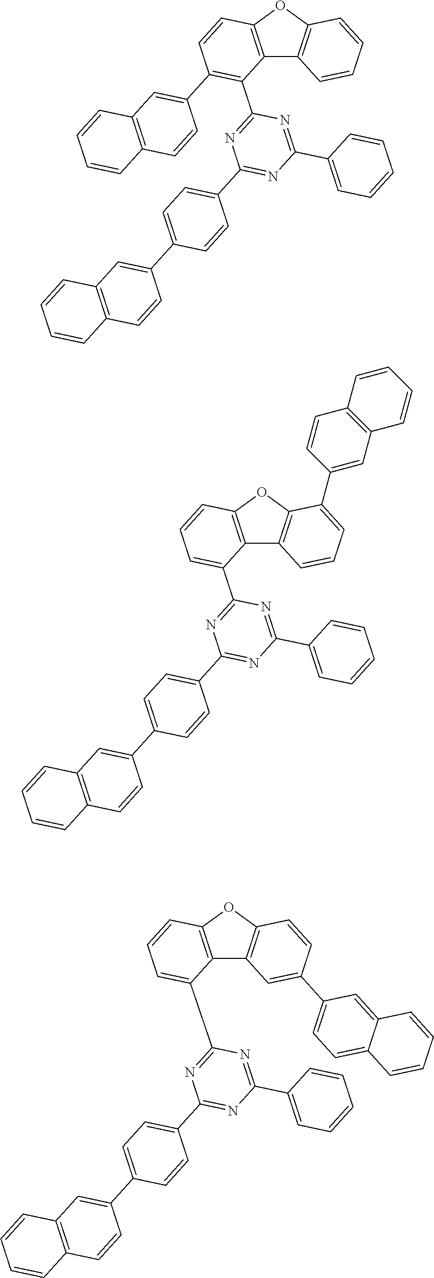 [SMILES: c1ccc(-c2nc(-c3ccc(-c4ccc5ccccc5c4)cc3)nc(-c3c(-c4ccc5ccccc5c4)ccc4oc5ccccc5c34)n2)cc1 ; c1ccc(-c2nc(-c3ccc(-c4ccc5ccccc5c4)cc3)nc(-c3cccc4oc5c(-c6ccc7ccccc7c6)cccc5c34)n2)cc1 ; c1ccc(-c2nc(-c3ccc(-c4ccc5ccccc5c4)cc3)nc(-c3cccc4oc5ccc(-c6ccc7ccccc7c6)cc5c34)n2)cc1]

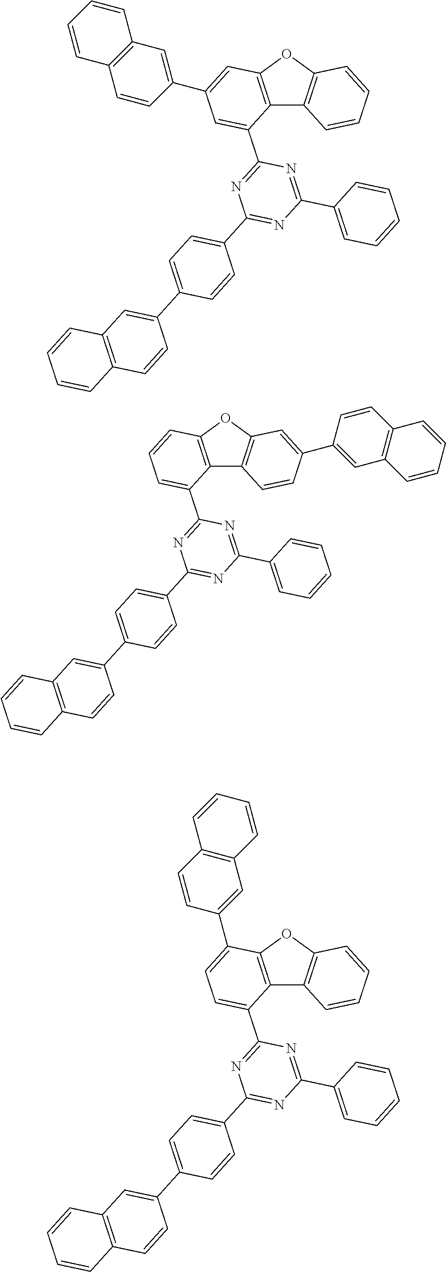 [SMILES: c1ccc(-c2nc(-c3ccc(-c4ccc5ccccc5c4)cc3)nc(-c3cc(-c4ccc5ccccc5c4)cc4oc5ccccc5c34)n2)cc1 ; c1ccc(-c2nc(-c3ccc(-c4ccc5ccccc5c4)cc3)nc(-c3cccc4oc5cc(-c6ccc7ccccc7c6)ccc5c34)n2)cc1 ; c1ccc(-c2nc(-c3ccc(-c4ccc5ccccc5c4)cc3)nc(-c3ccc(-c4ccc5ccccc5c4)c4oc5ccccc5c34)n2)cc1]

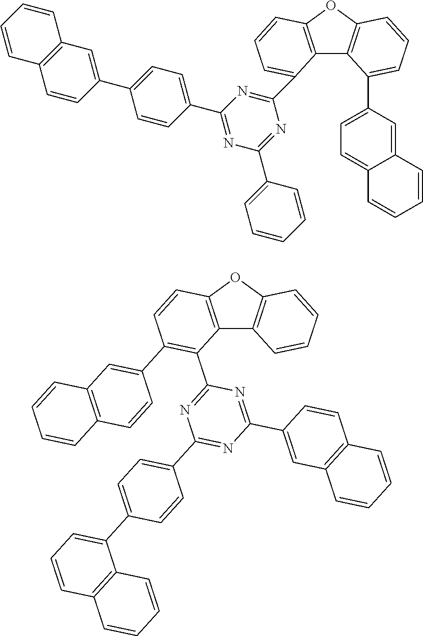 [SMILES: c1ccc(-c2nc(-c3ccc(-c4ccc5ccccc5c4)cc3)nc(-c3cccc4oc5cccc(-c6ccc7ccccc7c6)c5c34)n2)cc1 ; c1ccc2cc(-c3nc(-c4ccc(-c5cccc6ccccc56)cc4)nc(-c4c(-c5ccc6ccccc6c5)ccc5oc6ccccc6c45)n3)ccc2c1]

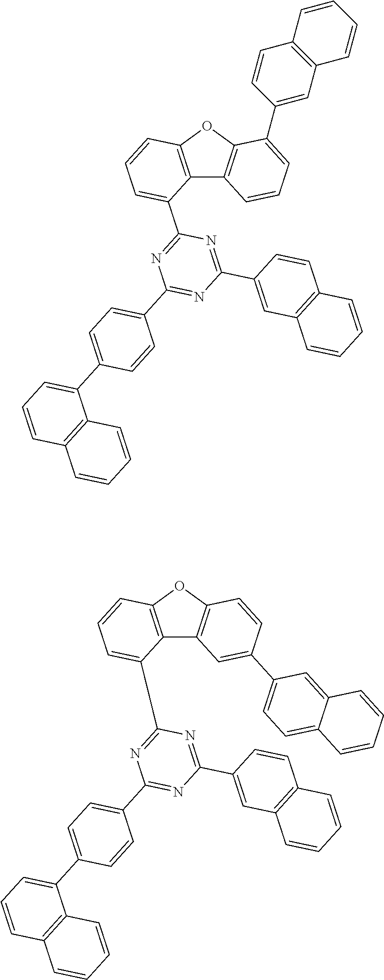 [SMILES: c1ccc2cc(-c3nc(-c4ccc(-c5cccc6ccccc56)cc4)nc(-c4cccc5oc6c(-c7ccc8ccccc8c7)cccc6c45)n3)ccc2c1 ; c1ccc2cc(-c3ccc4oc5cccc(-c6nc(-c7ccc(-c8cccc9ccccc89)cc7)nc(-c7ccc8ccccc8c7)n6)c5c4c3)ccc2c1]

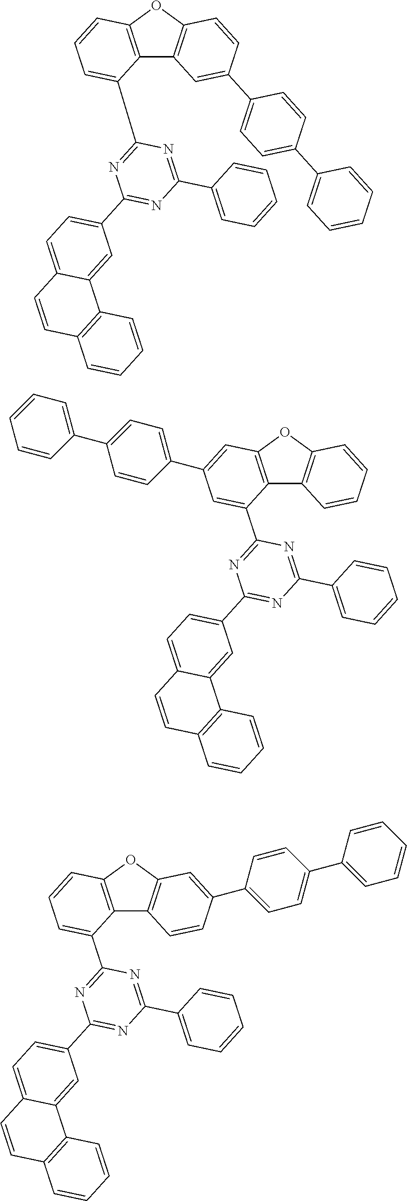

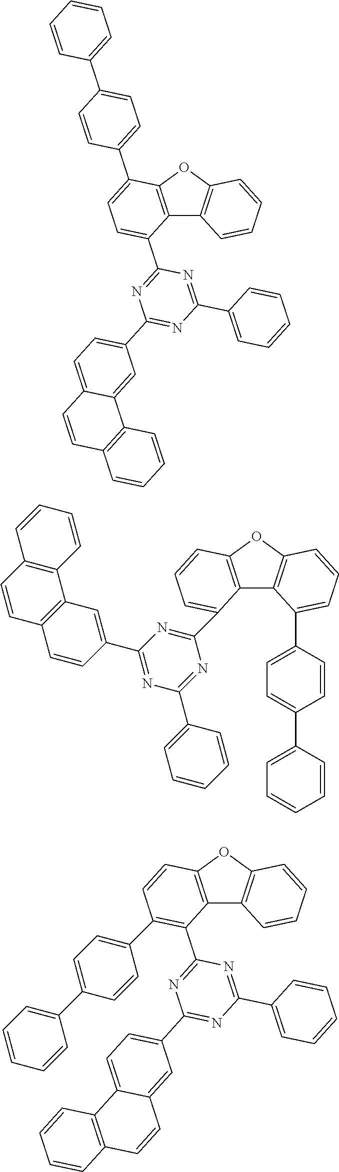 [SMILES: c1ccc(-c2ccc(-c3ccc(-c4nc(-c5ccccc5)nc(-c5ccc6ccc7ccccc7c6c5)n4)c4c3oc3ccccc34)cc2)cc1 ; c1ccc(-c2ccc(-c3cccc4oc5cccc(-c6nc(-c7ccccc7)nc(-c7ccc8ccc9ccccc9c8c7)n6)c5c34)cc2)cc1 ; c1ccc(-c2ccc(-c3ccc4oc5ccccc5c4c3-c3nc(-c4ccccc4)nc(-c4ccc5c(ccc6ccccc65)c4)n3)cc2)cc1]

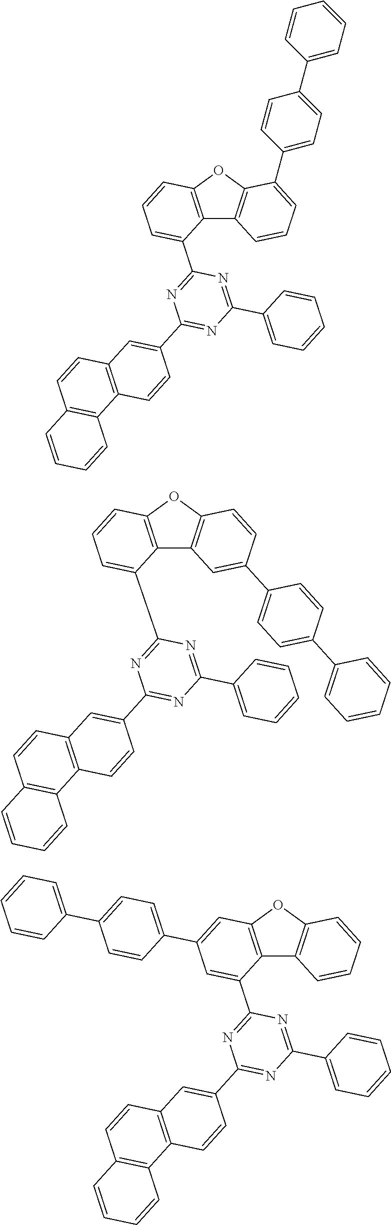

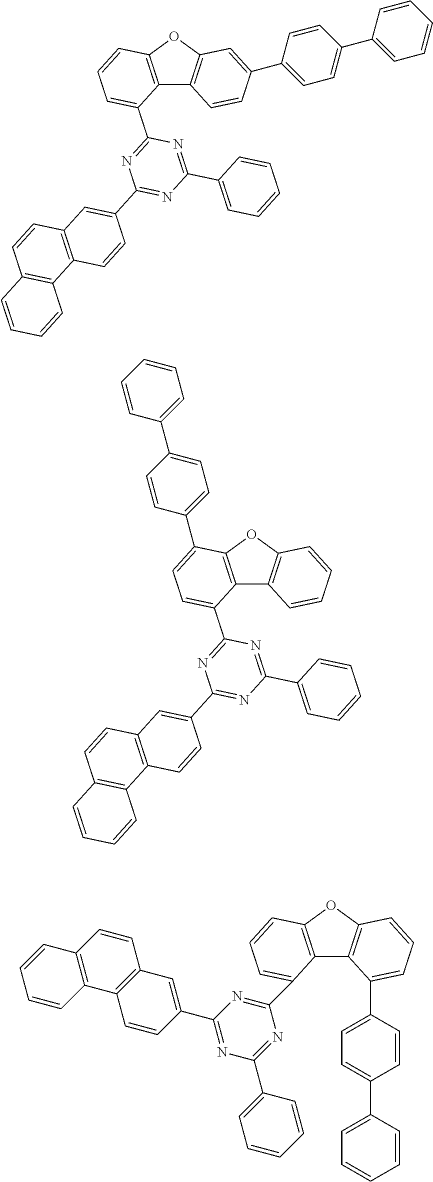 [SMILES: c1ccc(-c2ccc(-c3ccc4c(c3)oc3cccc(-c5nc(-c6ccccc6)nc(-c6ccc7c(ccc8ccccc87)c6)n5)c34)cc2)cc1 ; c1ccc(-c2ccc(-c3ccc(-c4nc(-c5ccccc5)nc(-c5ccc6c(ccc7ccccc76)c5)n4)c4c3oc3ccccc34)cc2)cc1 ; c1ccc(-c2ccc(-c3cccc4oc5cccc(-c6nc(-c7ccccc7)nc(-c7ccc8c(ccc9ccccc98)c7)n6)c5c34)cc2)cc1]

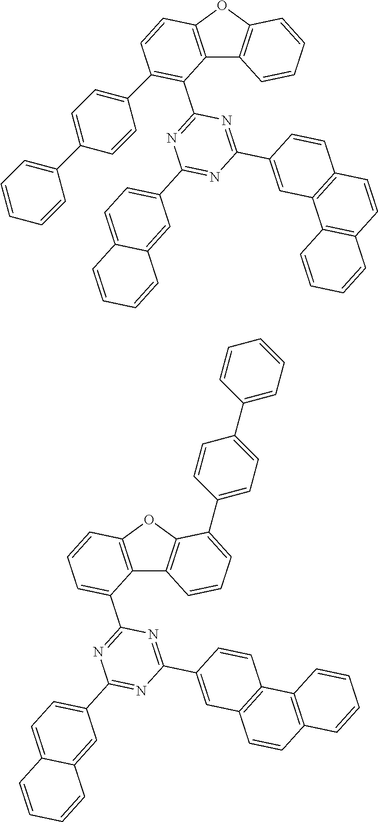 [SMILES: c1ccc(-c2ccc(-c3ccc4oc5ccccc5c4c3-c3nc(-c4ccc5ccccc5c4)nc(-c4ccc5ccc6ccccc6c5c4)n3)cc2)cc1 ; c1ccc(-c2ccc(-c3cccc4c3oc3cccc(-c5nc(-c6ccc7ccccc7c6)nc(-c6ccc7c(ccc8ccccc87)c6)n5)c34)cc2)cc1]

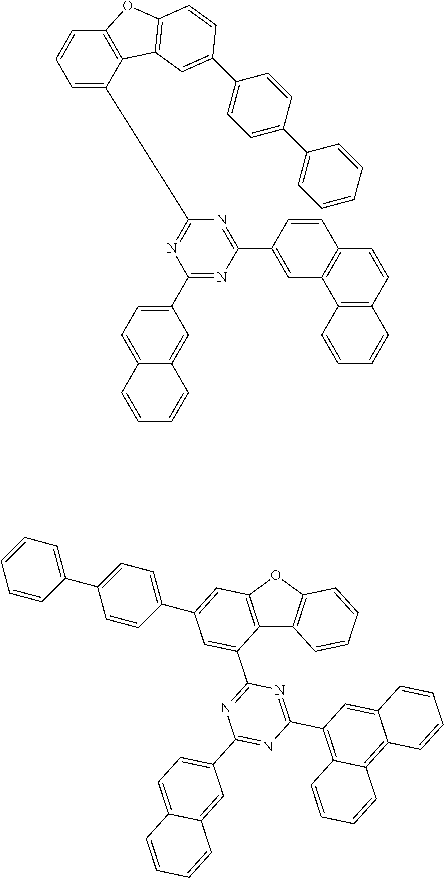 [SMILES: c1ccc(-c2ccc(-c3ccc4oc5cccc(-c6nc(-c7ccc8ccccc8c7)nc(-c7ccc8ccc9ccccc9c8c7)n6)c5c4c3)cc2)cc1 ; c1ccc(-c2ccc(-c3cc(-c4nc(-c5ccc6ccccc6c5)nc(-c5cc6ccccc6c6ccccc56)n4)c4c(c3)oc3ccccc34)cc2)cc1]

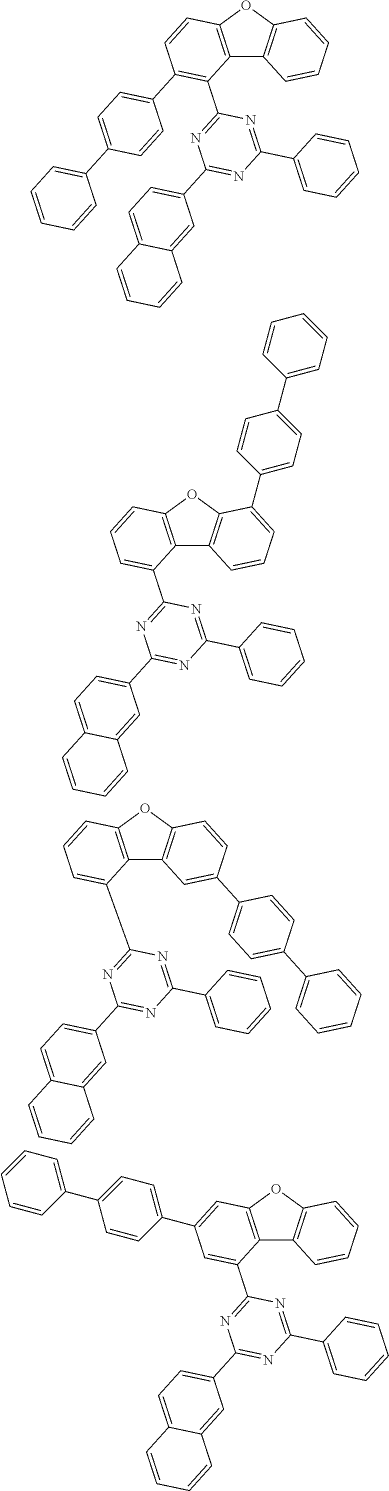

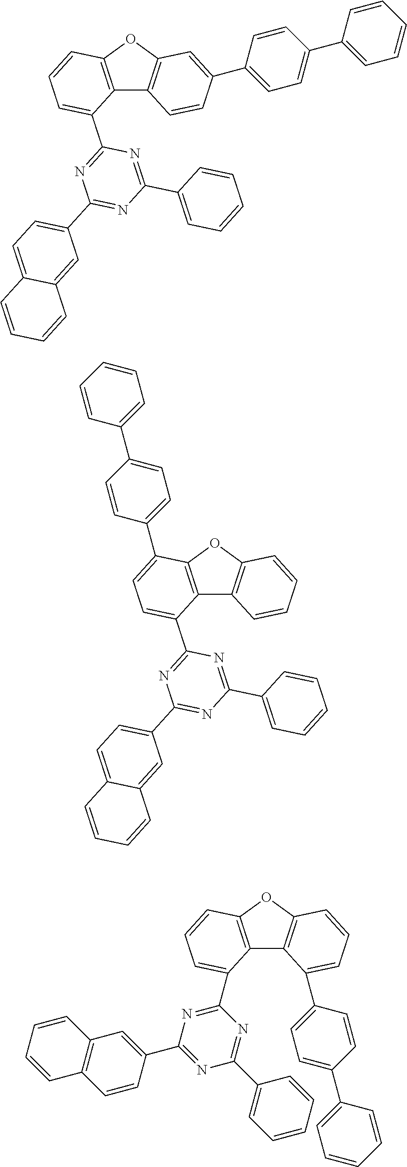 [SMILES: c1ccc(-c2ccc(-c3ccc4c(c3)oc3cccc(-c5nc(-c6ccccc6)nc(-c6ccc7ccccc7c6)n5)c34)cc2)cc1 ; c1ccc(-c2ccc(-c3ccc(-c4nc(-c5ccccc5)nc(-c5ccc6ccccc6c5)n4)c4c3oc3ccccc34)cc2)cc1 ; c1ccc(-c2ccc(-c3cccc4oc5cccc(-c6nc(-c7ccccc7)nc(-c7ccc8ccccc8c7)n6)c5c34)cc2)cc1]

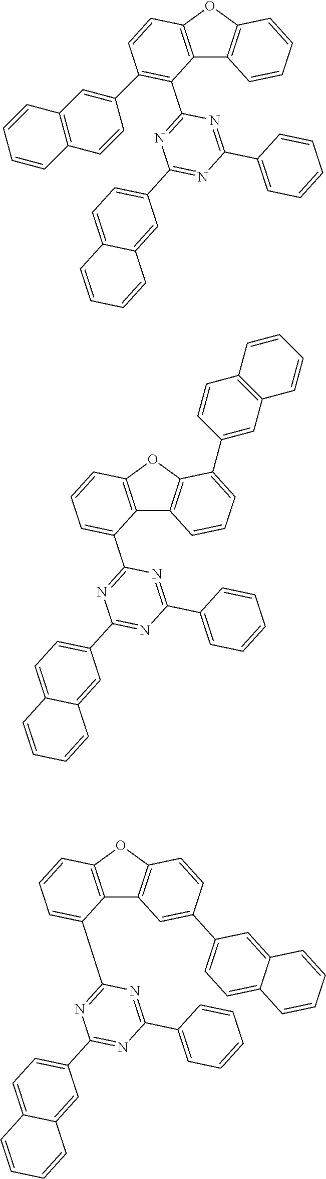 [SMILES: c1ccc(-c2nc(-c3ccc4ccccc4c3)nc(-c3c(-c4ccc5ccccc5c4)ccc4oc5ccccc5c34)n2)cc1 ; c1ccc(-c2nc(-c3ccc4ccccc4c3)nc(-c3cccc4oc5c(-c6ccc7ccccc7c6)cccc5c34)n2)cc1 ; c1ccc(-c2nc(-c3ccc4ccccc4c3)nc(-c3cccc4oc5ccc(-c6ccc7ccccc7c6)cc5c34)n2)cc1]

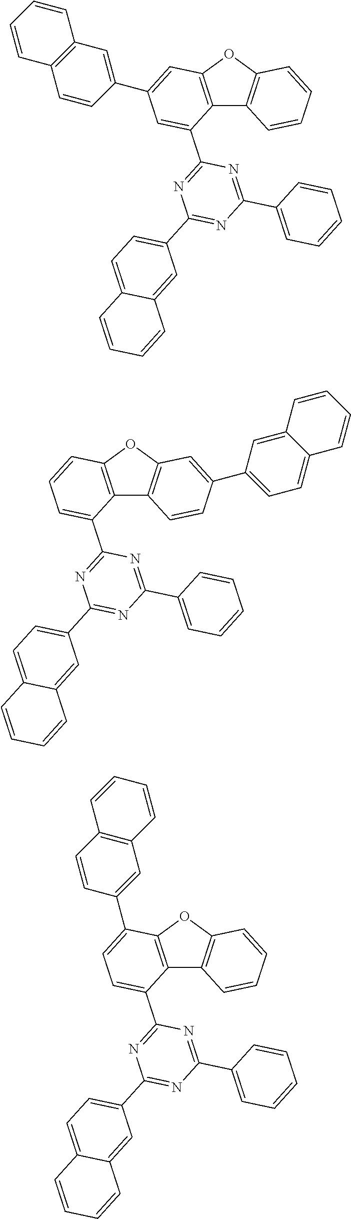 [SMILES: c1ccc(-c2nc(-c3ccc4ccccc4c3)nc(-c3cc(-c4ccc5ccccc5c4)cc4oc5ccccc5c34)n2)cc1 ; c1ccc(-c2nc(-c3ccc4ccccc4c3)nc(-c3cccc4oc5cc(-c6ccc7ccccc7c6)ccc5c34)n2)cc1 ; c1ccc(-c2nc(-c3ccc4ccccc4c3)nc(-c3ccc(-c4ccc5ccccc5c4)c4oc5ccccc5c34)n2)cc1]

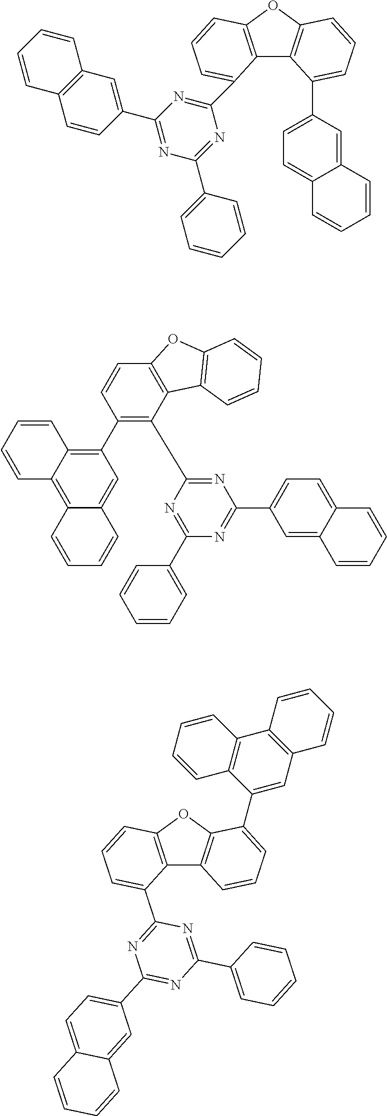 [SMILES: c1ccc(-c2nc(-c3ccc4ccccc4c3)nc(-c3cccc4oc5cccc(-c6ccc7ccccc7c6)c5c34)n2)cc1 ; c1ccc(-c2nc(-c3ccc4ccccc4c3)nc(-c3c(-c4cc5ccccc5c5ccccc45)ccc4oc5ccccc5c34)n2)cc1 ; c1ccc(-c2nc(-c3ccc4ccccc4c3)nc(-c3cccc4oc5c(-c6cc7ccccc7c7ccccc67)cccc5c34)n2)cc1]

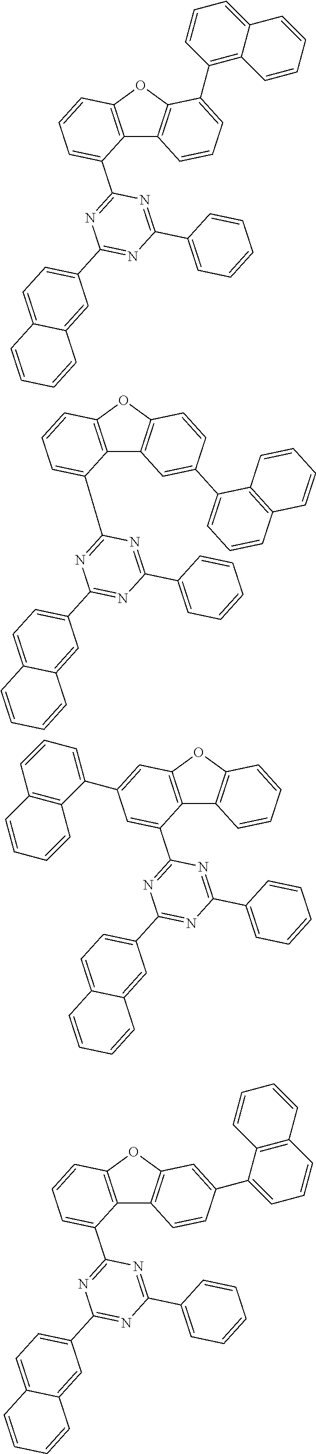

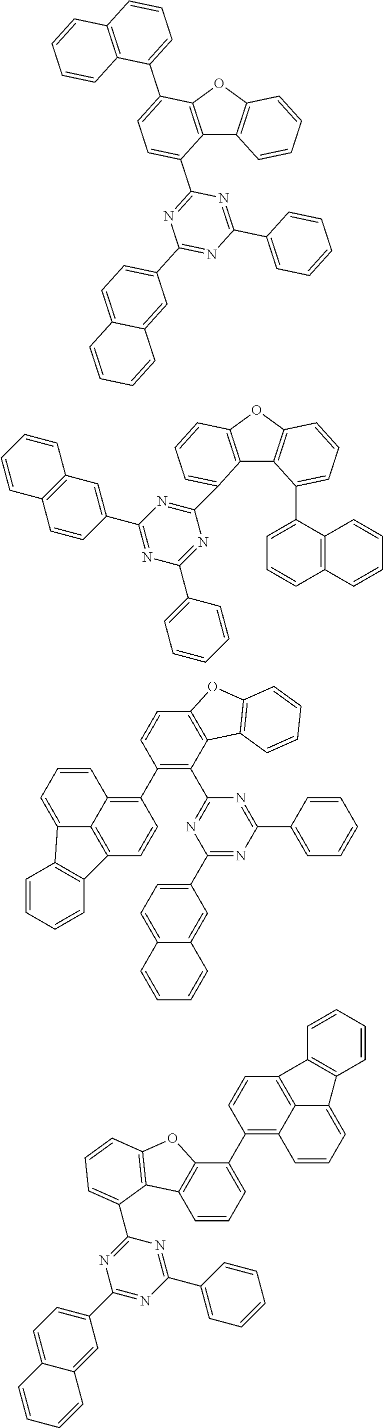

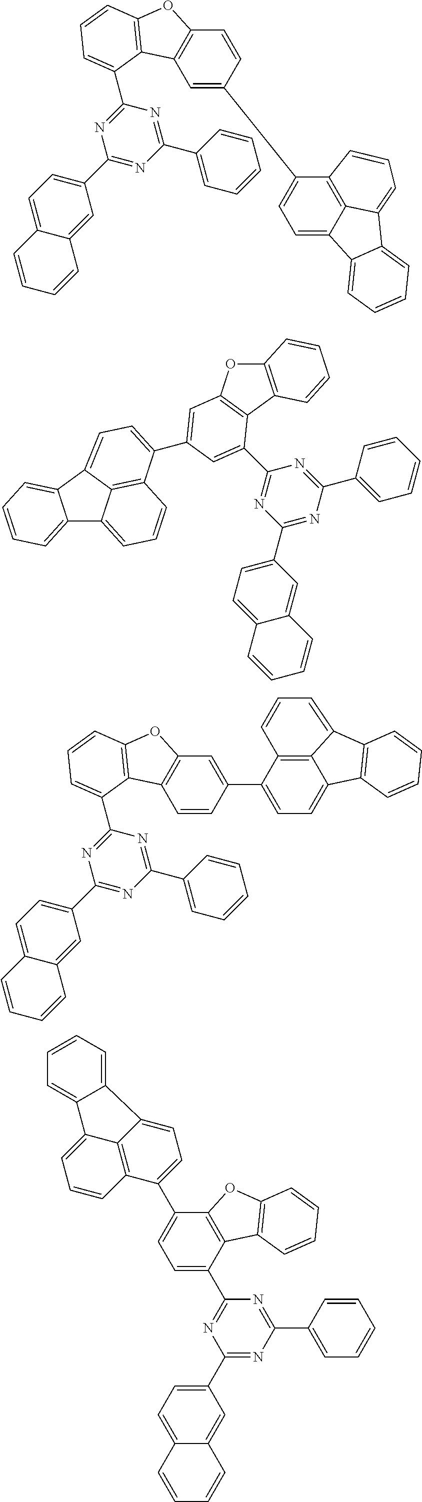

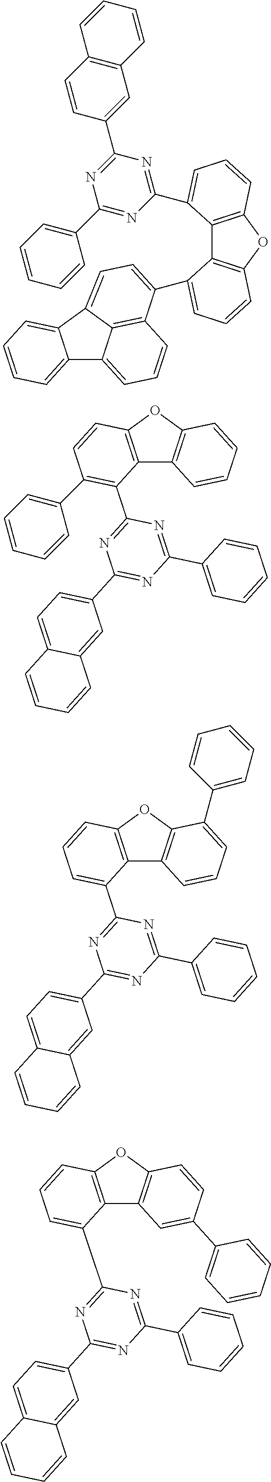

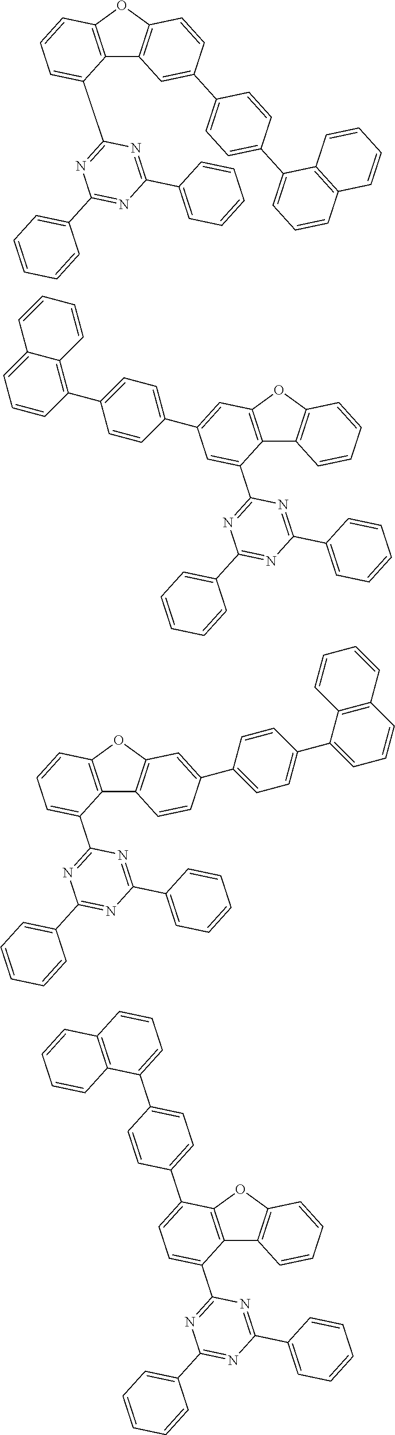

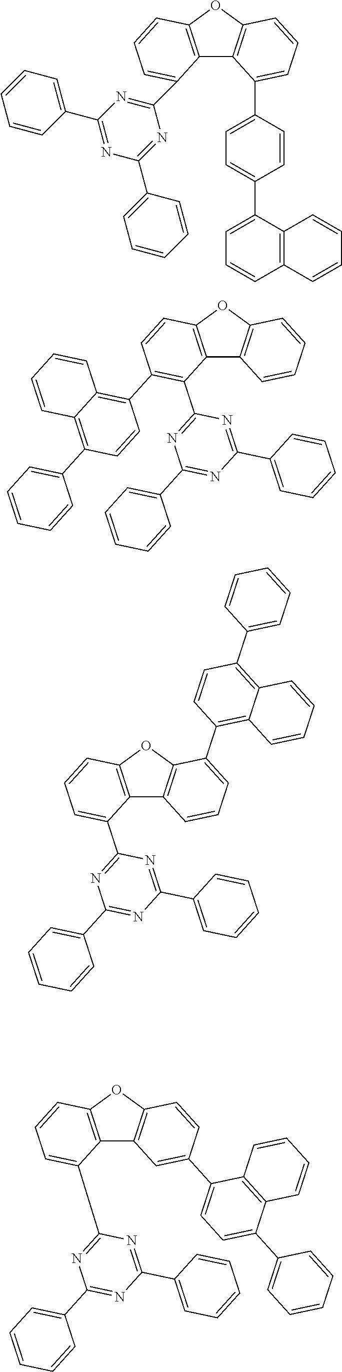

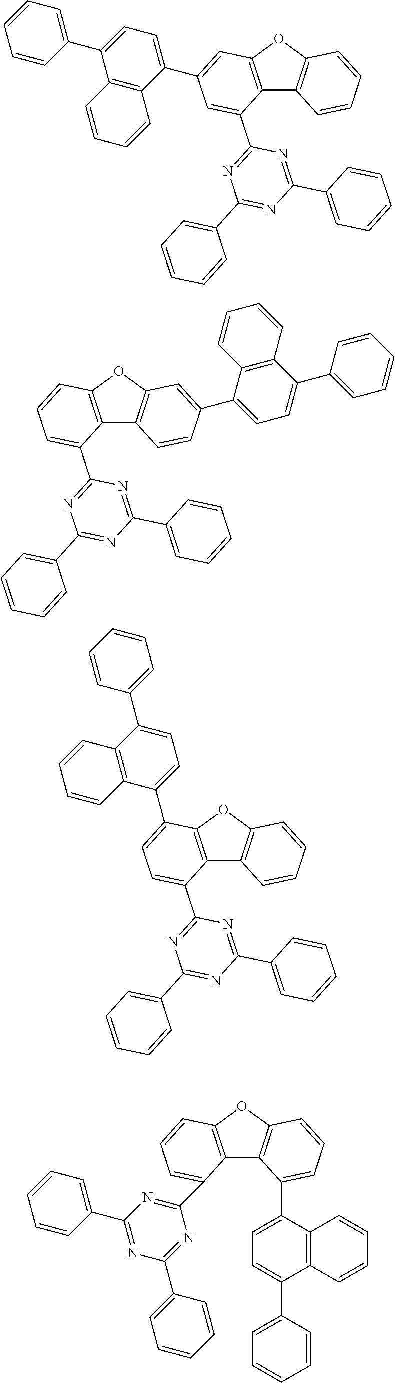

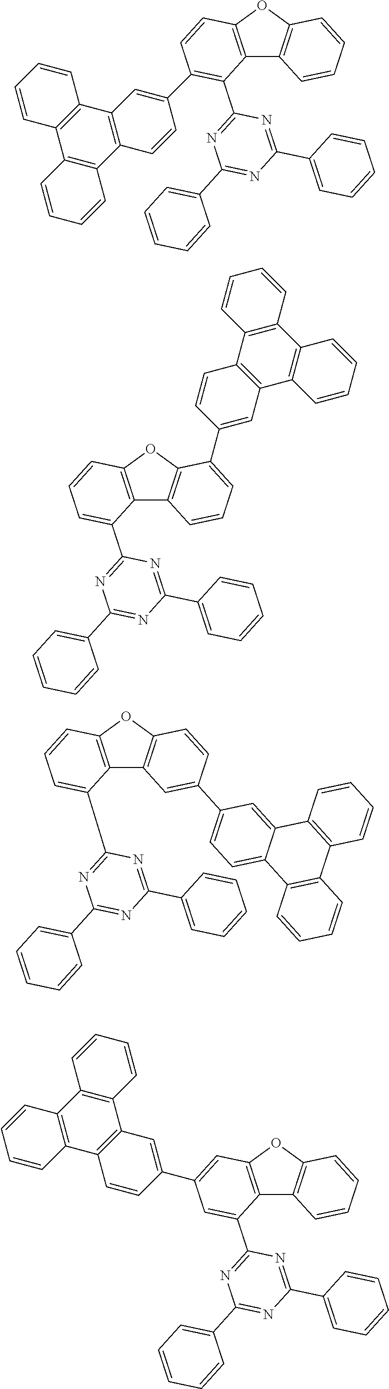

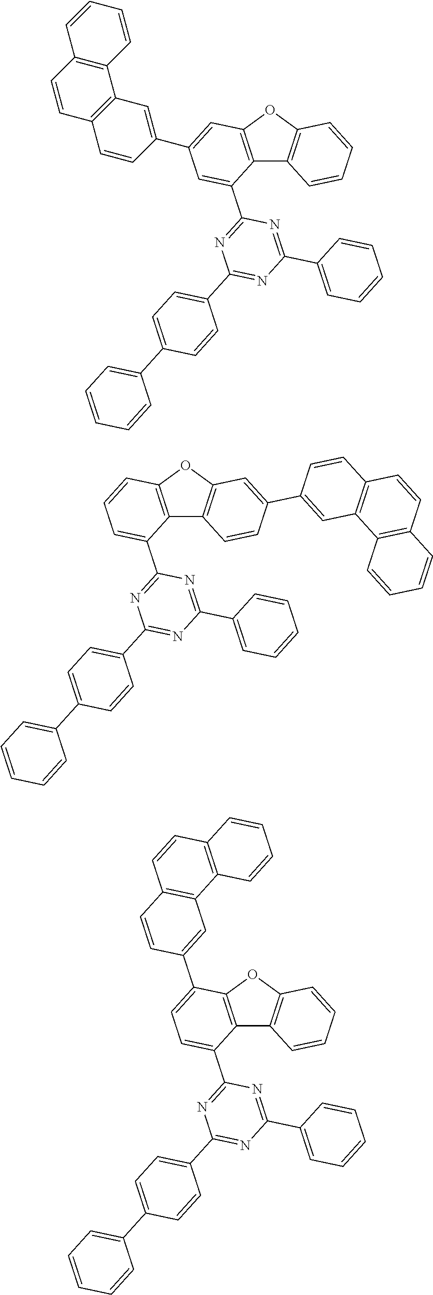 [SMILES: c1ccc(-c2ccc(-c3nc(-c4ccccc4)nc(-c4cc(-c5ccc6ccc7ccccc7c6c5)cc5oc6ccccc6c45)n3)cc2)cc1 ; c1ccc(-c2ccc(-c3nc(-c4ccccc4)nc(-c4cccc5oc6cc(-c7ccc8ccc9ccccc9c8c7)ccc6c45)n3)cc2)cc1 ; c1ccc(-c2ccc(-c3nc(-c4ccccc4)nc(-c4ccc(-c5ccc6ccc7ccccc7c6c5)c5oc6ccccc6c45)n3)cc2)cc1]

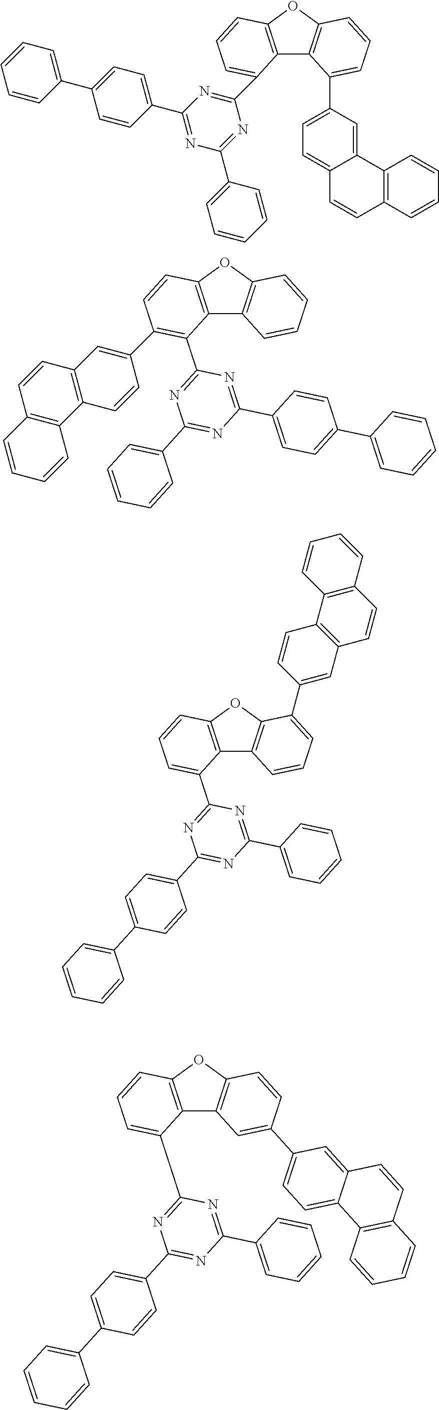

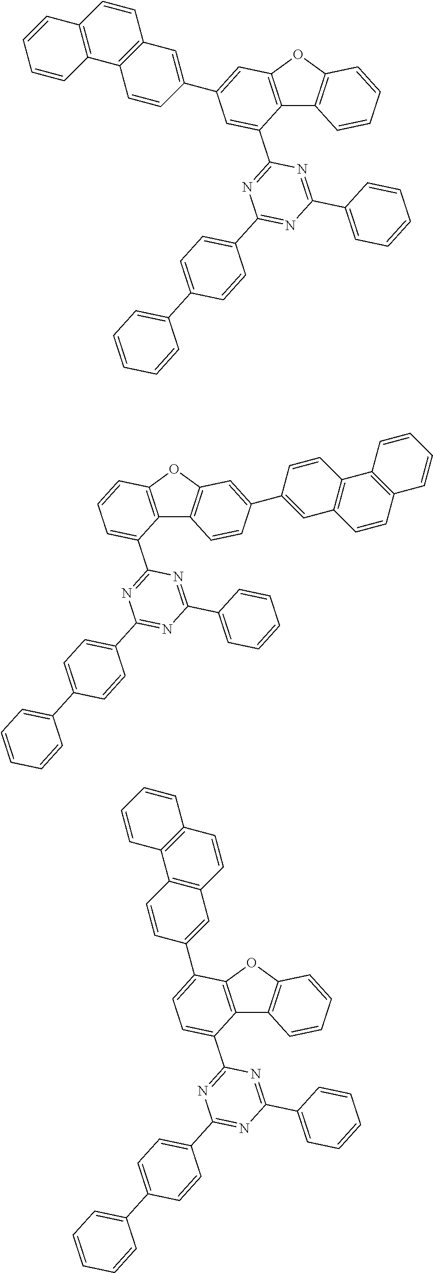 [SMILES: c1ccc(-c2ccc(-c3nc(-c4ccccc4)nc(-c4cc(-c5ccc6c(ccc7ccccc76)c5)cc5oc6ccccc6c45)n3)cc2)cc1 ; c1ccc(-c2ccc(-c3nc(-c4ccccc4)nc(-c4cccc5oc6cc(-c7ccc8c(ccc9ccccc98)c7)ccc6c45)n3)cc2)cc1 ; c1ccc(-c2ccc(-c3nc(-c4ccccc4)nc(-c4ccc(-c5ccc6c(ccc7ccccc76)c5)c5oc6ccccc6c45)n3)cc2)cc1]

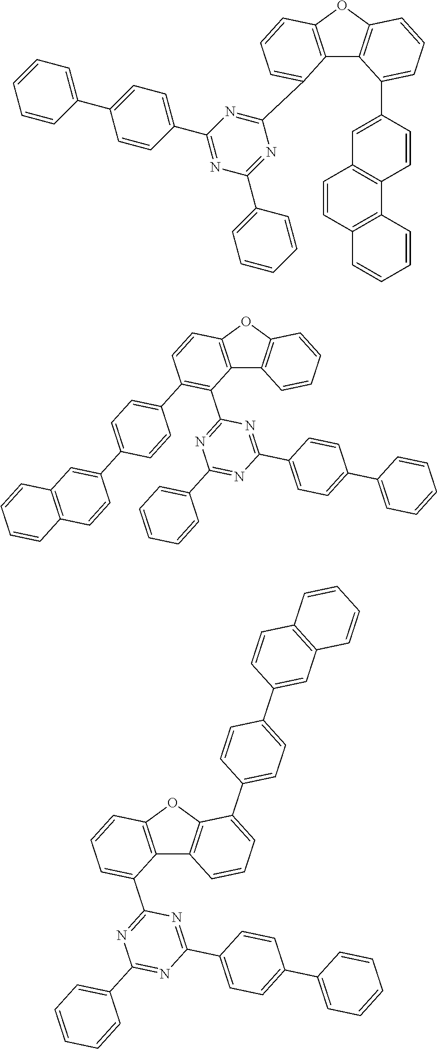 [SMILES: c1ccc(-c2ccc(-c3nc(-c4ccccc4)nc(-c4cccc5oc6cccc(-c7ccc8c(ccc9ccccc98)c7)c6c45)n3)cc2)cc1 ; c1ccc(-c2ccc(-c3nc(-c4ccccc4)nc(-c4c(-c5ccc(-c6ccc7ccccc7c6)cc5)ccc5oc6ccccc6c45)n3)cc2)cc1 ; c1ccc(-c2ccc(-c3nc(-c4ccccc4)nc(-c4cccc5oc6c(-c7ccc(-c8ccc9ccccc9c8)cc7)cccc6c45)n3)cc2)cc1]

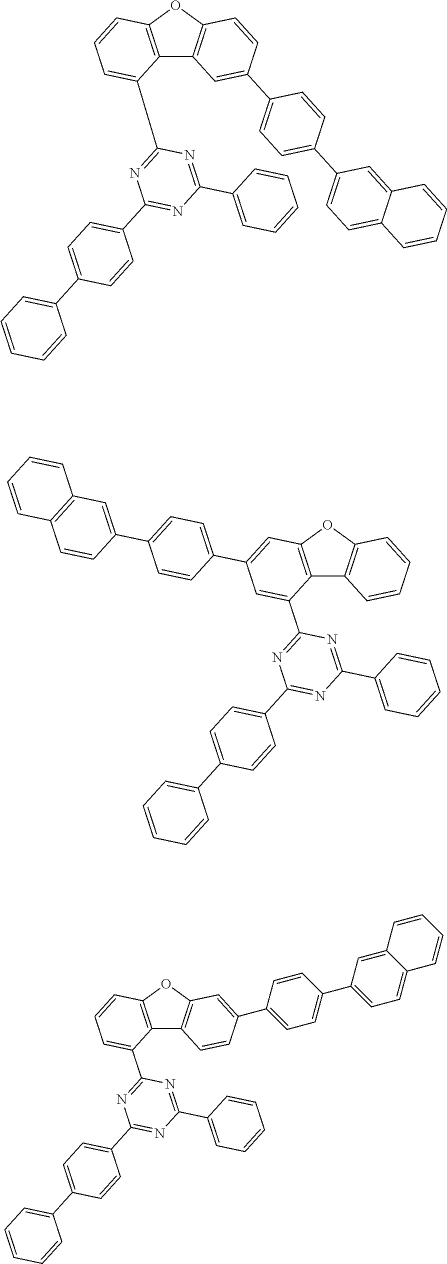 [SMILES: c1ccc(-c2ccc(-c3nc(-c4ccccc4)nc(-c4cccc5oc6ccc(-c7ccc(-c8ccc9ccccc9c8)cc7)cc6c45)n3)cc2)cc1 ; c1ccc(-c2ccc(-c3nc(-c4ccccc4)nc(-c4cc(-c5ccc(-c6ccc7ccccc7c6)cc5)cc5oc6ccccc6c45)n3)cc2)cc1 ; c1ccc(-c2ccc(-c3nc(-c4ccccc4)nc(-c4cccc5oc6cc(-c7ccc(-c8ccc9ccccc9c8)cc7)ccc6c45)n3)cc2)cc1]

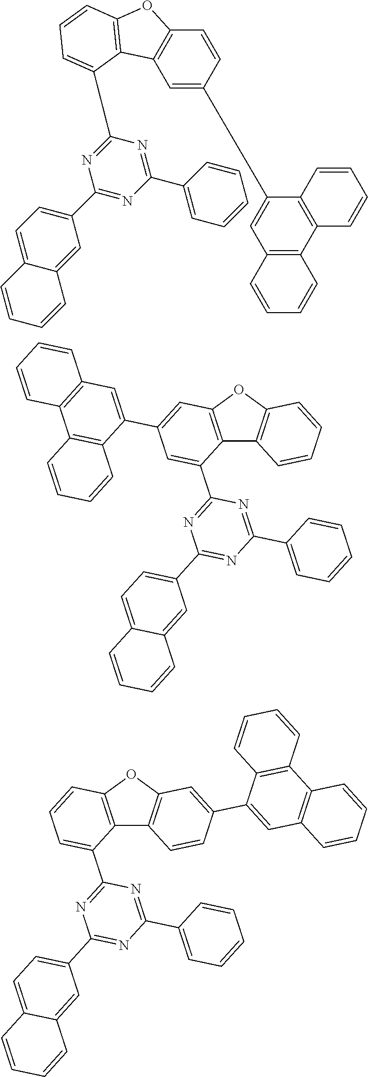

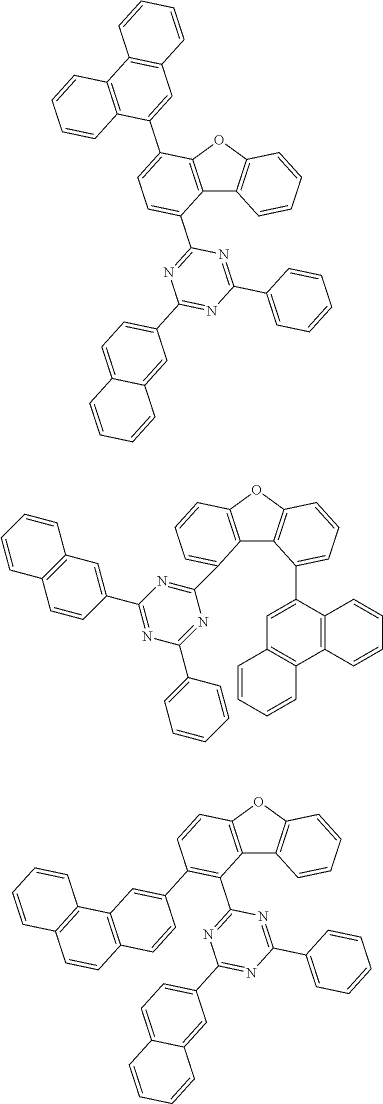 [SMILES: c1ccc(-c2nc(-c3ccc4ccccc4c3)nc(-c3ccc(-c4cc5ccccc5c5ccccc45)c4oc5ccccc5c34)n2)cc1 ; c1ccc(-c2nc(-c3ccc4ccccc4c3)nc(-c3cccc4oc5cccc(-c6cc7ccccc7c7ccccc67)c5c34)n2)cc1 ; c1ccc(-c2nc(-c3ccc4ccccc4c3)nc(-c3c(-c4ccc5ccc6ccccc6c5c4)ccc4oc5ccccc5c34)n2)cc1]

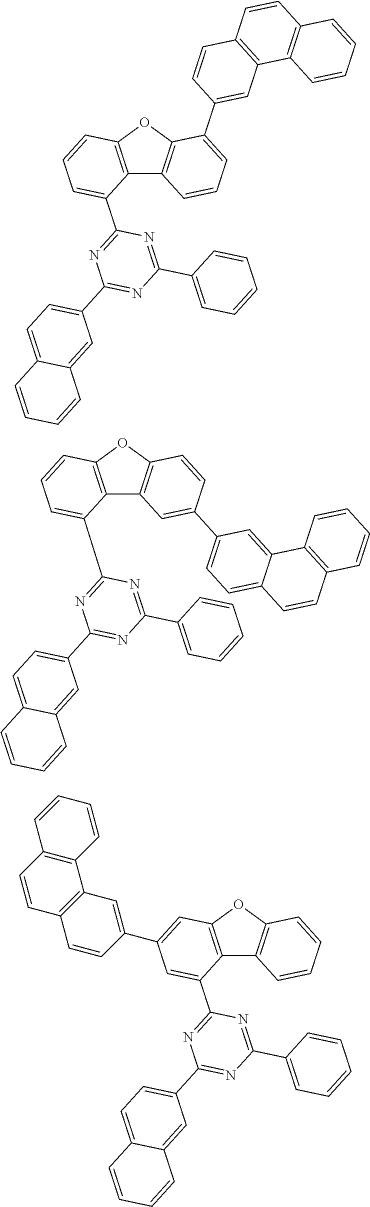

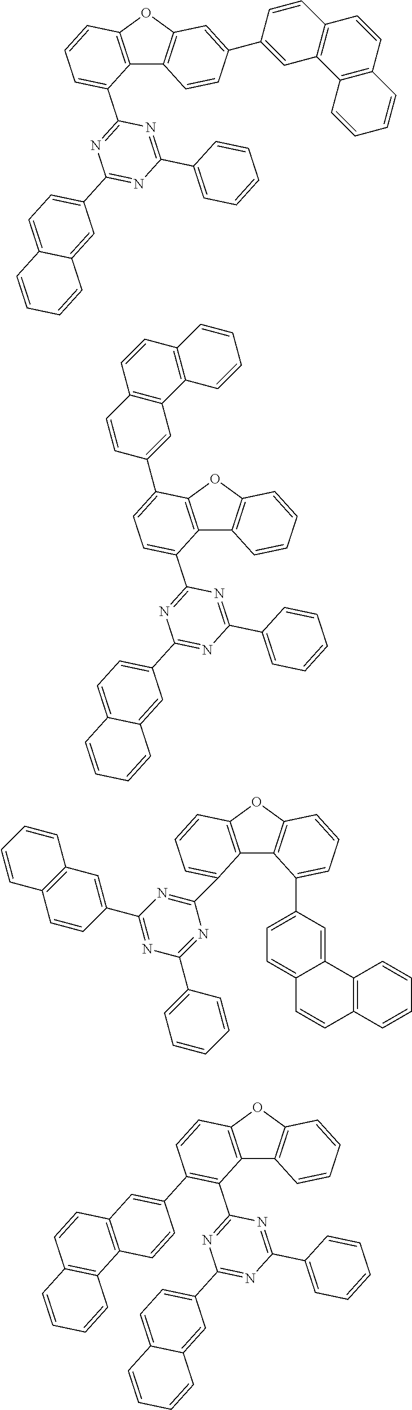 [SMILES: c1ccc(-c2nc(-c3ccc4ccccc4c3)nc(-c3cccc4oc5cc(-c6ccc7ccc8ccccc8c7c6)ccc5c34)n2)cc1 ; c1ccc(-c2nc(-c3ccc4ccccc4c3)nc(-c3ccc(-c4ccc5ccc6ccccc6c5c4)c4oc5ccccc5c34)n2)cc1 ; c1ccc(-c2nc(-c3ccc4ccccc4c3)nc(-c3cccc4oc5cccc(-c6ccc7ccc8ccccc8c7c6)c5c34)n2)cc1 ; c1ccc(-c2nc(-c3ccc4ccccc4c3)nc(-c3c(-c4ccc5c(ccc6ccccc65)c4)ccc4oc5ccccc5c34)n2)cc1]

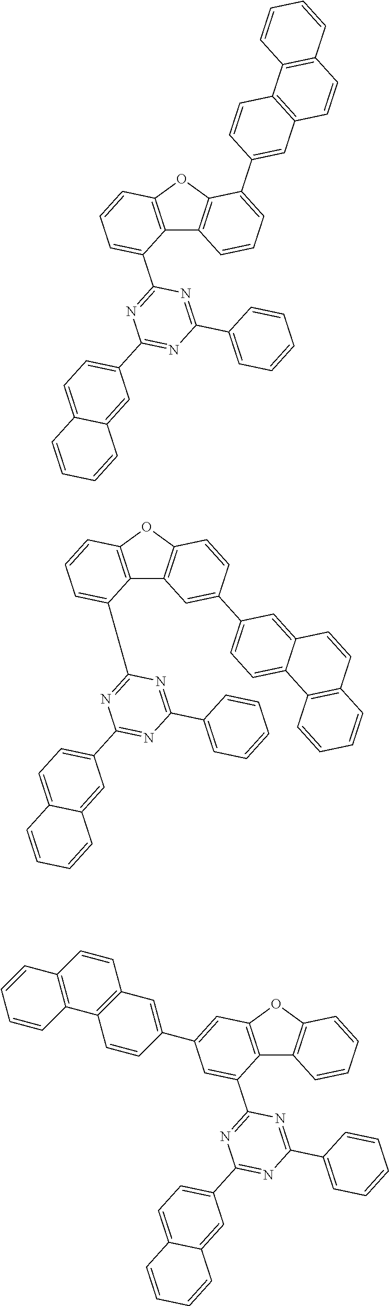 [SMILES: c1ccc(-c2nc(-c3ccc4ccccc4c3)nc(-c3cccc4oc5c(-c6ccc7c(ccc8ccccc87)c6)cccc5c34)n2)cc1 ; c1ccc(-c2nc(-c3ccc4ccccc4c3)nc(-c3cccc4oc5ccc(-c6ccc7c(ccc8ccccc87)c6)cc5c34)n2)cc1 ; c1ccc(-c2nc(-c3ccc4ccccc4c3)nc(-c3cc(-c4ccc5c(ccc6ccccc65)c4)cc4oc5ccccc5c34)n2)cc1]

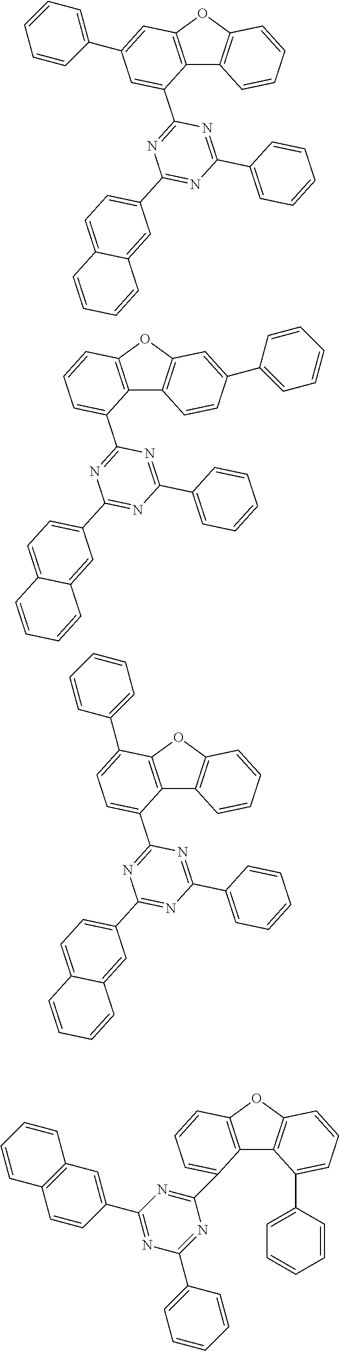

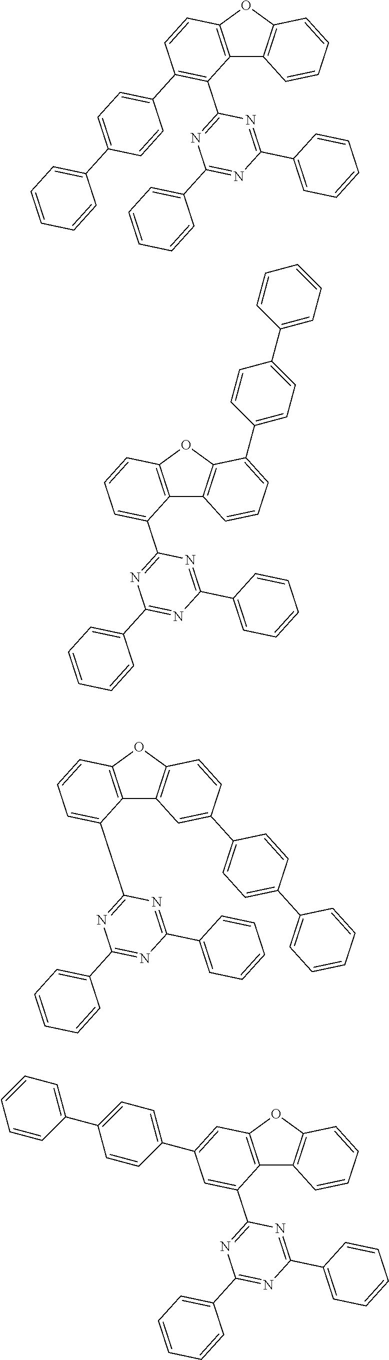 [SMILES: c1ccc(-c2ccc(-c3ccc4oc5ccccc5c4c3-c3nc(-c4ccccc4)nc(-c4ccccc4)n3)cc2)cc1 ; c1ccc(-c2ccc(-c3cccc4c3oc3cccc(-c5nc(-c6ccccc6)nc(-c6ccccc6)n5)c34)cc2)cc1 ; c1ccc(-c2ccc(-c3ccc4oc5cccc(-c6nc(-c7ccccc7)nc(-c7ccccc7)n6)c5c4c3)cc2)cc1 ; c1ccc(-c2ccc(-c3cc(-c4nc(-c5ccccc5)nc(-c5ccccc5)n4)c4c(c3)oc3ccccc34)cc2)cc1]

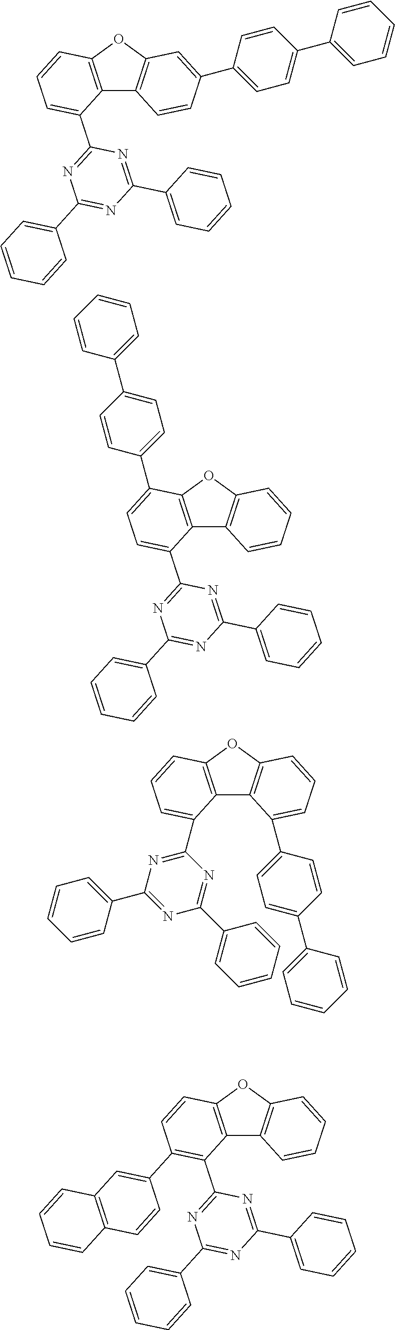

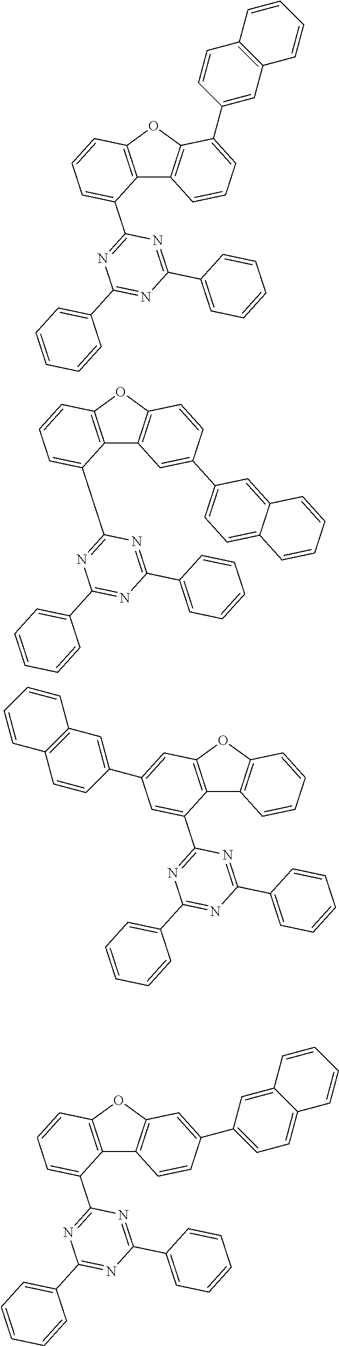

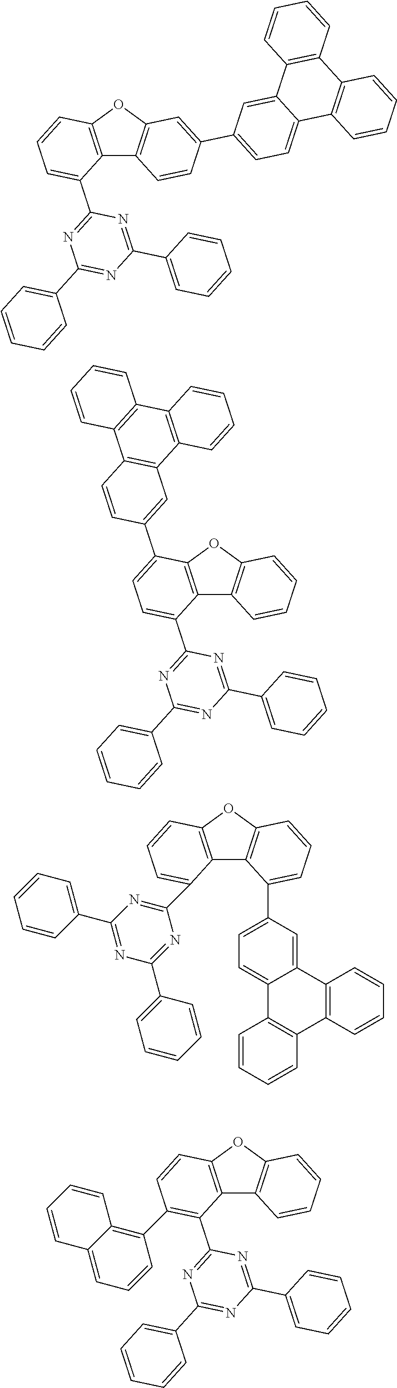

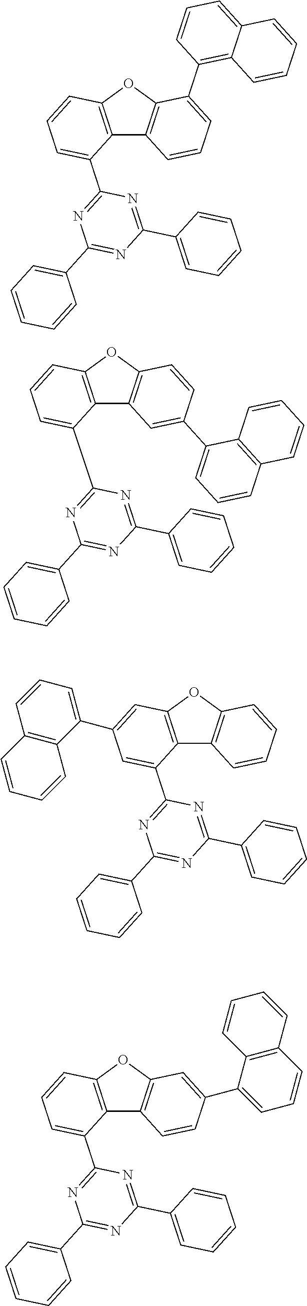

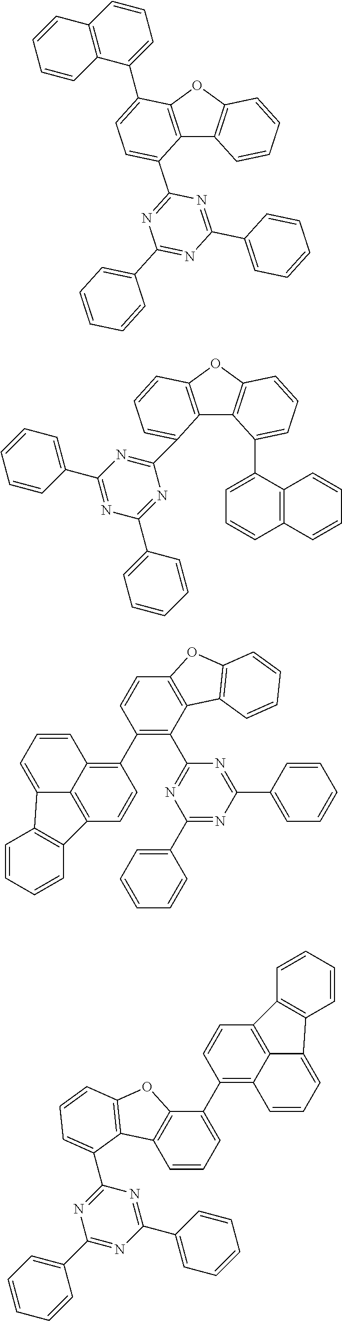

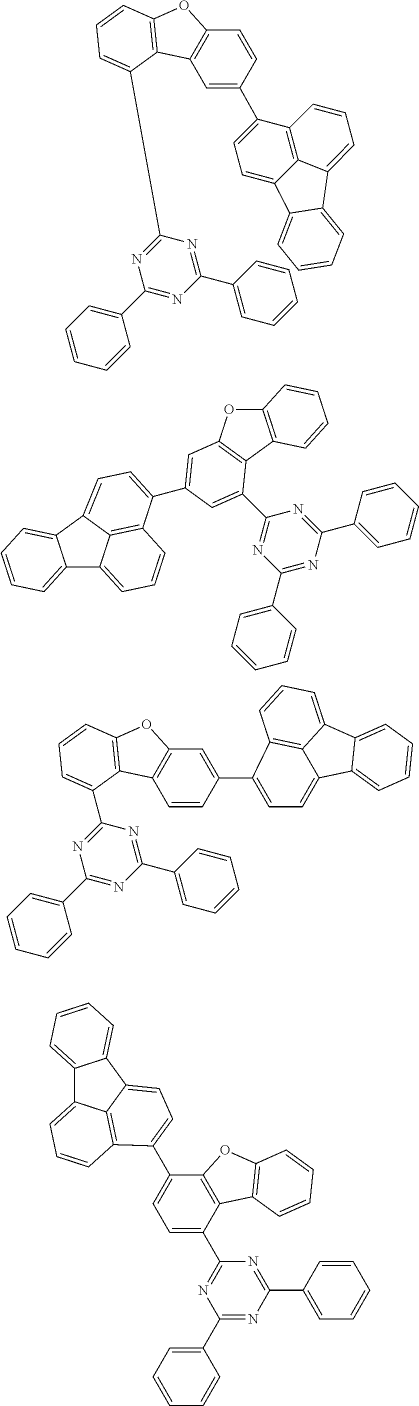

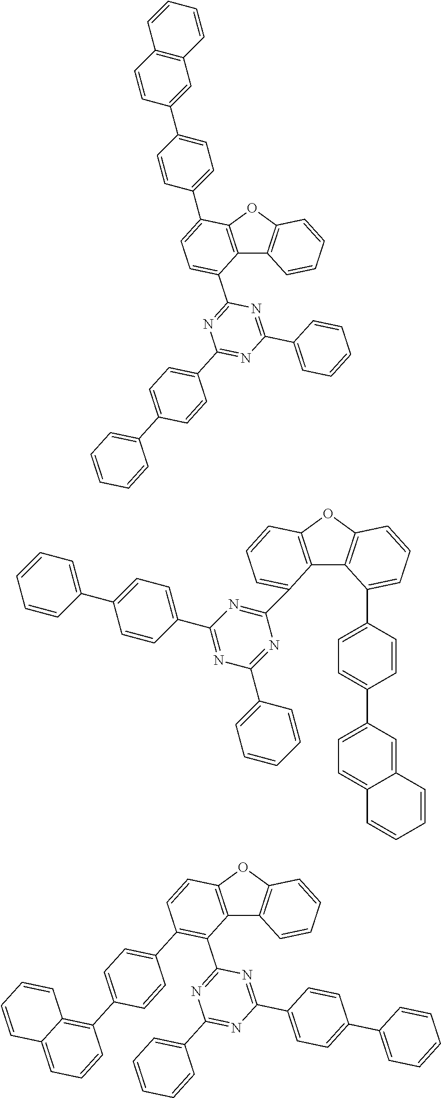 [SMILES: c1ccc(-c2ccc(-c3nc(-c4ccccc4)nc(-c4ccc(-c5ccc(-c6ccc7ccccc7c6)cc5)c5oc6ccccc6c45)n3)cc2)cc1 ; c1ccc(-c2ccc(-c3nc(-c4ccccc4)nc(-c4cccc5oc6cccc(-c7ccc(-c8ccc9ccccc9c8)cc7)c6c45)n3)cc2)cc1 ; c1ccc(-c2ccc(-c3nc(-c4ccccc4)nc(-c4c(-c5ccc(-c6cccc7ccccc67)cc5)ccc5oc6ccccc6c45)n3)cc2)cc1]

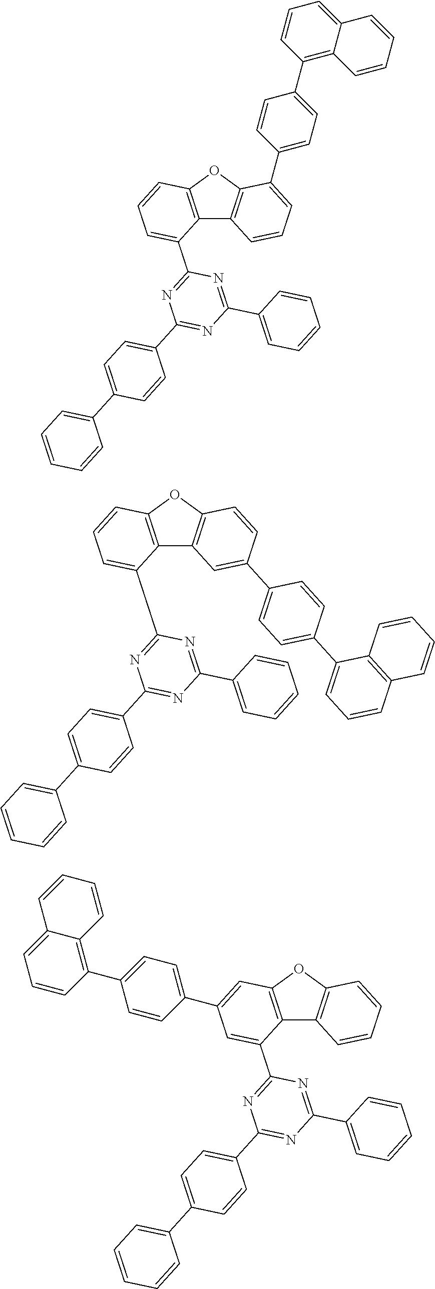

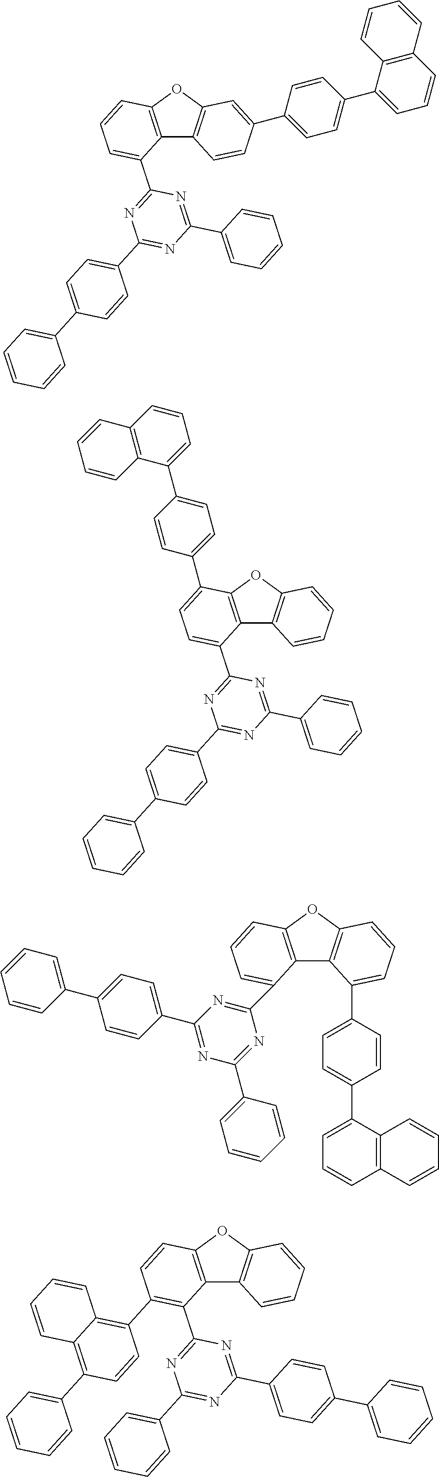 [SMILES: c1ccc(-c2ccc(-c3nc(-c4ccccc4)nc(-c4cccc5oc6cc(-c7ccc(-c8cccc9ccccc89)cc7)ccc6c45)n3)cc2)cc1 ; c1ccc(-c2ccc(-c3nc(-c4ccccc4)nc(-c4ccc(-c5ccc(-c6cccc7ccccc67)cc5)c5oc6ccccc6c45)n3)cc2)cc1 ; c1ccc(-c2ccc(-c3nc(-c4ccccc4)nc(-c4cccc5oc6cccc(-c7ccc(-c8cccc9ccccc89)cc7)c6c45)n3)cc2)cc1 ; c1ccc(-c2ccc(-c3nc(-c4ccccc4)nc(-c4c(-c5ccc(-c6ccccc6)c6ccccc56)ccc5oc6ccccc6c45)n3)cc2)cc1]

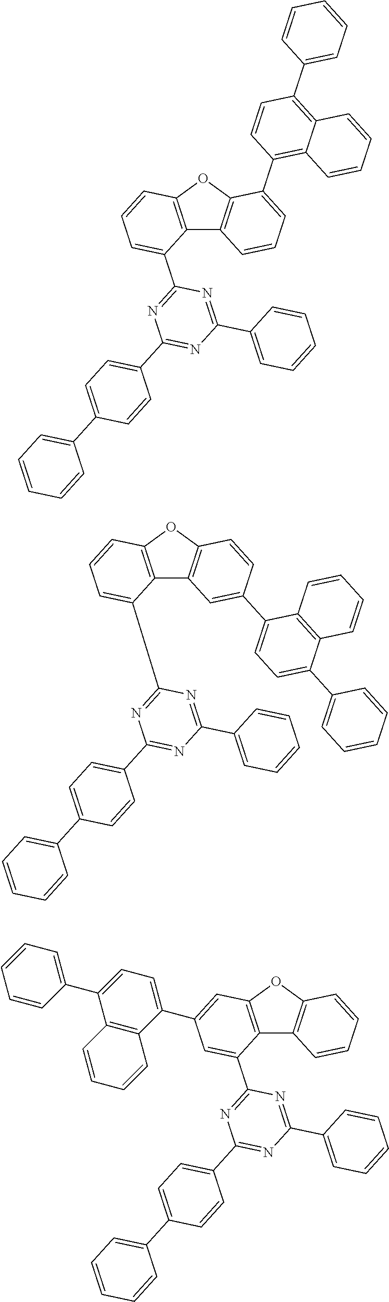 [SMILES: c1ccc(-c2ccc(-c3nc(-c4ccccc4)nc(-c4cccc5oc6c(-c7ccc(-c8ccccc8)c8ccccc78)cccc6c45)n3)cc2)cc1 ; c1ccc(-c2ccc(-c3nc(-c4ccccc4)nc(-c4cccc5oc6ccc(-c7ccc(-c8ccccc8)c8ccccc78)cc6c45)n3)cc2)cc1 ; c1ccc(-c2ccc(-c3nc(-c4ccccc4)nc(-c4cc(-c5ccc(-c6ccccc6)c6ccccc56)cc5oc6ccccc6c45)n3)cc2)cc1]

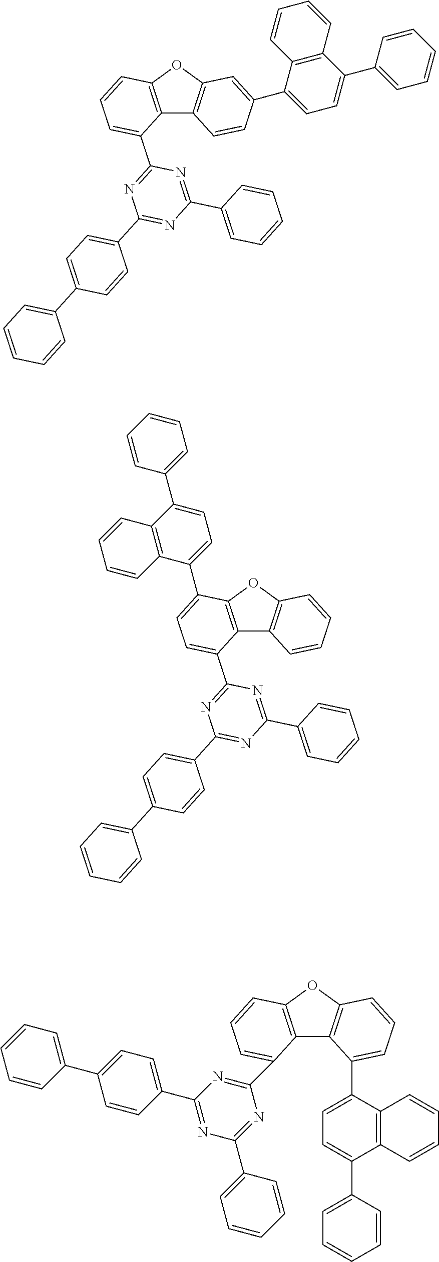 [SMILES: c1ccc(-c2ccc(-c3nc(-c4ccccc4)nc(-c4cccc5oc6cc(-c7ccc(-c8ccccc8)c8ccccc78)ccc6c45)n3)cc2)cc1 ; c1ccc(-c2ccc(-c3nc(-c4ccccc4)nc(-c4ccc(-c5ccc(-c6ccccc6)c6ccccc56)c5oc6ccccc6c45)n3)cc2)cc1 ; c1ccc(-c2ccc(-c3nc(-c4ccccc4)nc(-c4cccc5oc6cccc(-c7ccc(-c8ccccc8)c8ccccc78)c6c45)n3)cc2)cc1]

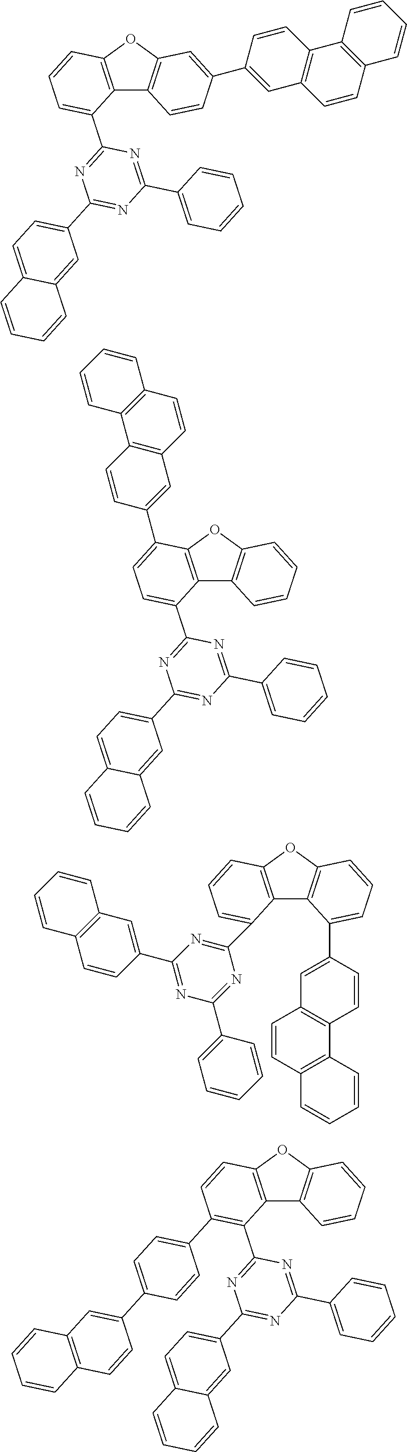

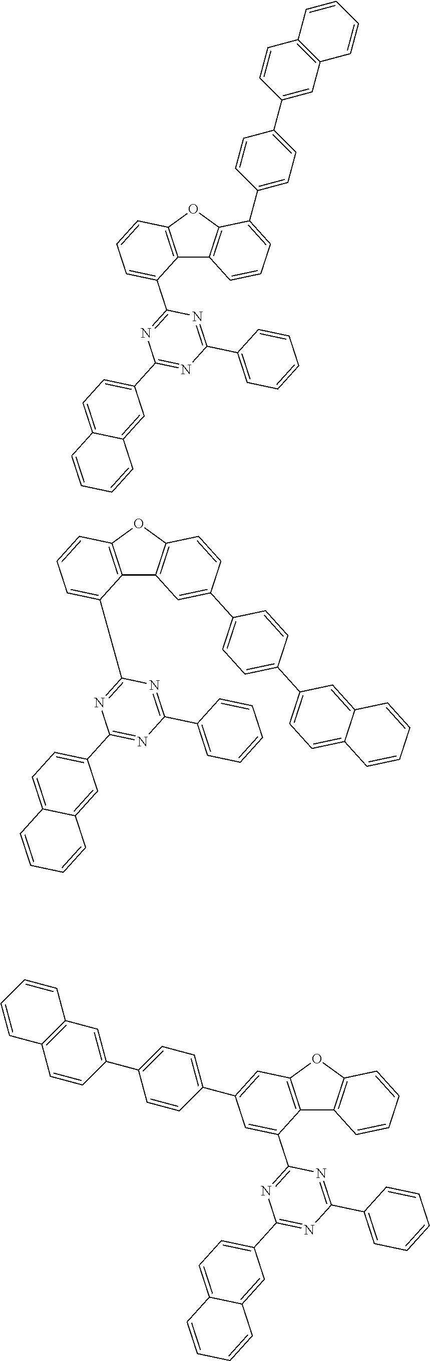

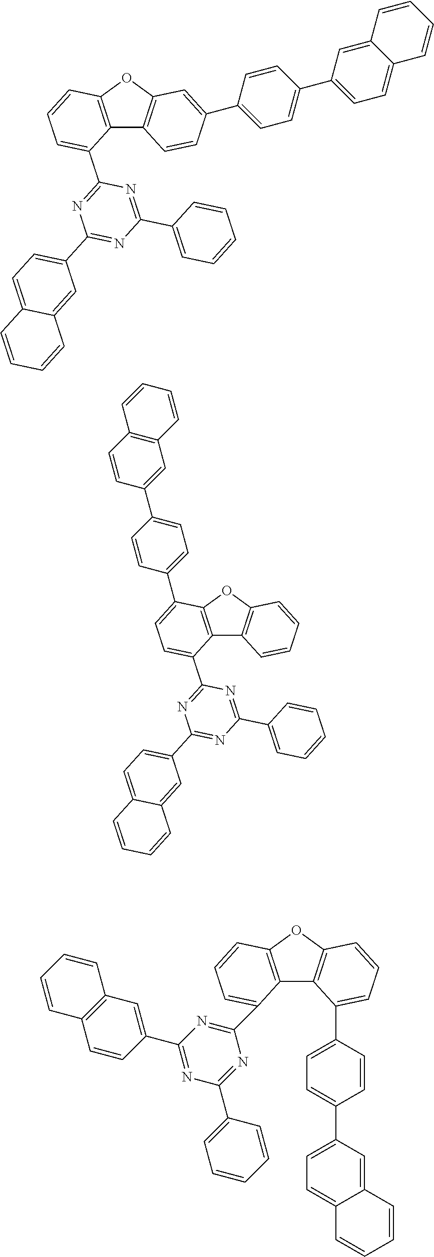 [SMILES: c1ccc(-c2nc(-c3ccc4ccccc4c3)nc(-c3cccc4oc5cc(-c6ccc(-c7ccc8ccccc8c7)cc6)ccc5c34)n2)cc1 ; c1ccc(-c2nc(-c3ccc4ccccc4c3)nc(-c3ccc(-c4ccc(-c5ccc6ccccc6c5)cc4)c4oc5ccccc5c34)n2)cc1 ; c1ccc(-c2nc(-c3ccc4ccccc4c3)nc(-c3cccc4oc5cccc(-c6ccc(-c7ccc8ccccc8c7)cc6)c5c34)n2)cc1]

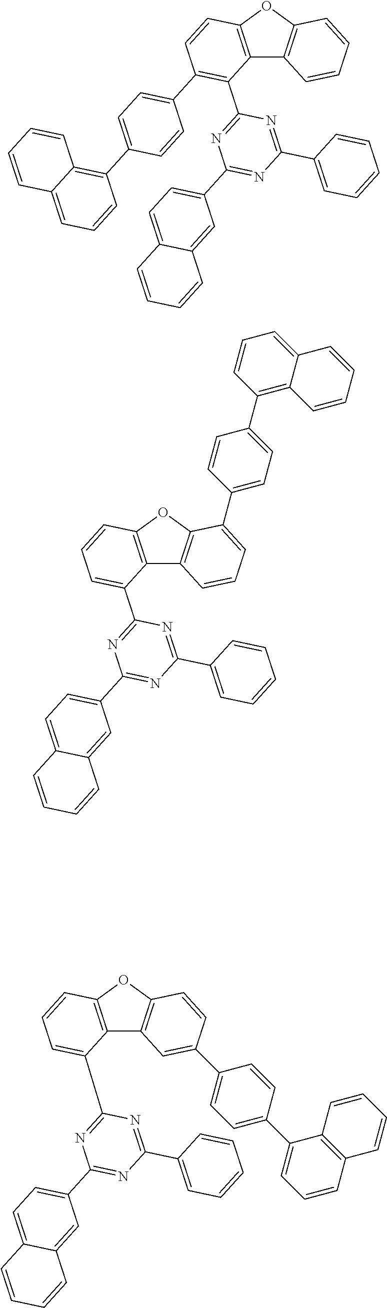 [SMILES: c1ccc(-c2nc(-c3ccc4ccccc4c3)nc(-c3c(-c4ccc(-c5cccc6ccccc56)cc4)ccc4oc5ccccc5c34)n2)cc1 ; c1ccc(-c2nc(-c3ccc4ccccc4c3)nc(-c3cccc4oc5c(-c6ccc(-c7cccc8ccccc78)cc6)cccc5c34)n2)cc1 ; c1ccc(-c2nc(-c3ccc4ccccc4c3)nc(-c3cccc4oc5ccc(-c6ccc(-c7cccc8ccccc78)cc6)cc5c34)n2)cc1]

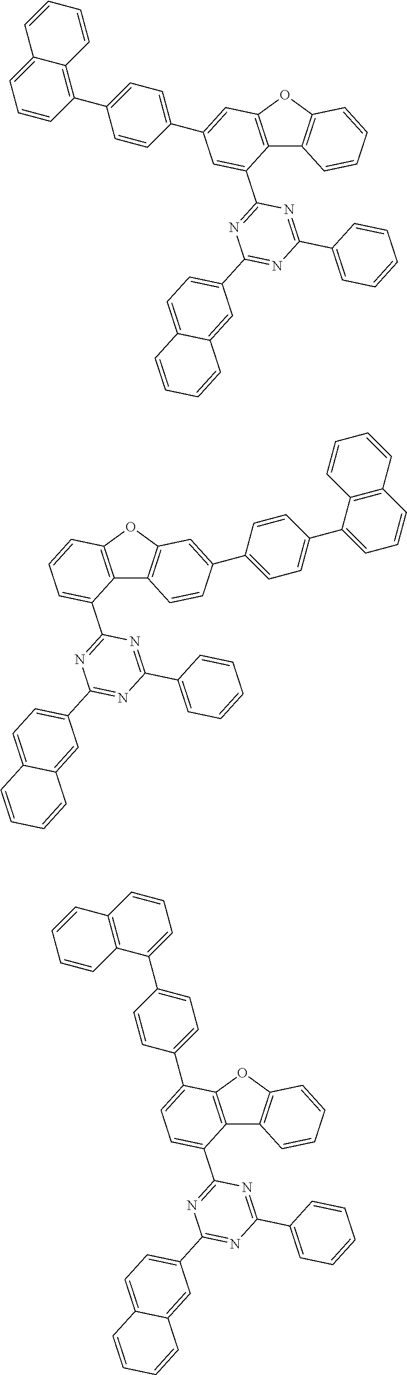 [SMILES: c1ccc(-c2nc(-c3ccc4ccccc4c3)nc(-c3cc(-c4ccc(-c5cccc6ccccc56)cc4)cc4oc5ccccc5c34)n2)cc1 ; c1ccc(-c2nc(-c3ccc4ccccc4c3)nc(-c3cccc4oc5cc(-c6ccc(-c7cccc8ccccc78)cc6)ccc5c34)n2)cc1 ; c1ccc(-c2nc(-c3ccc4ccccc4c3)nc(-c3ccc(-c4ccc(-c5cccc6ccccc56)cc4)c4oc5ccccc5c34)n2)cc1]

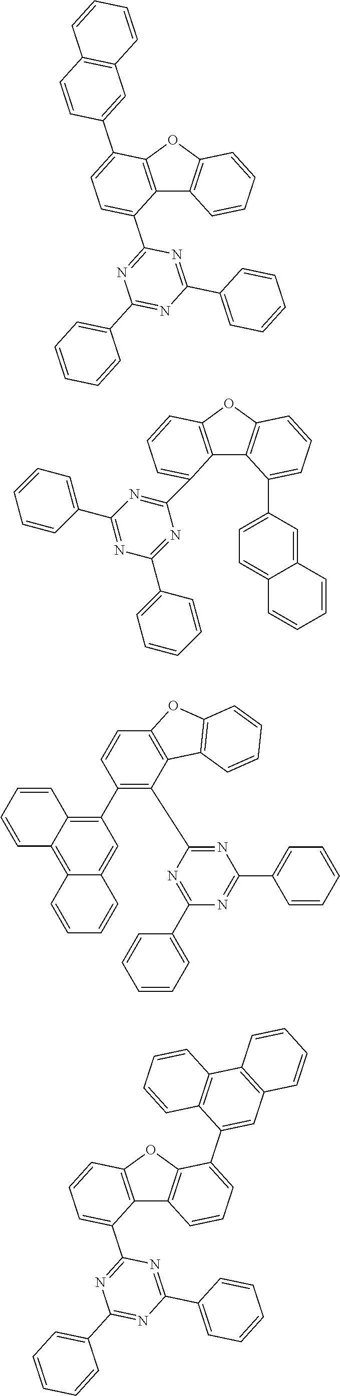 [SMILES: c1ccc(-c2nc(-c3ccccc3)nc(-c3ccc(-c4ccc5ccccc5c4)c4oc5ccccc5c34)n2)cc1 ; c1ccc(-c2nc(-c3ccccc3)nc(-c3cccc4oc5cccc(-c6ccc7ccccc7c6)c5c34)n2)cc1 ; c1ccc(-c2nc(-c3ccccc3)nc(-c3c(-c4cc5ccccc5c5ccccc45)ccc4oc5ccccc5c34)n2)cc1 ; c1ccc(-c2nc(-c3ccccc3)nc(-c3cccc4oc5c(-c6cc7ccccc7c7ccccc67)cccc5c34)n2)cc1]

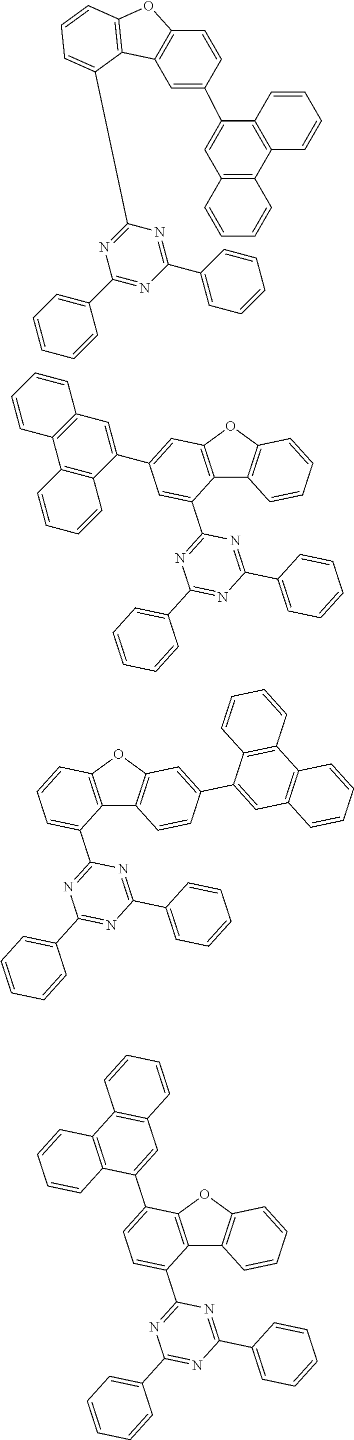

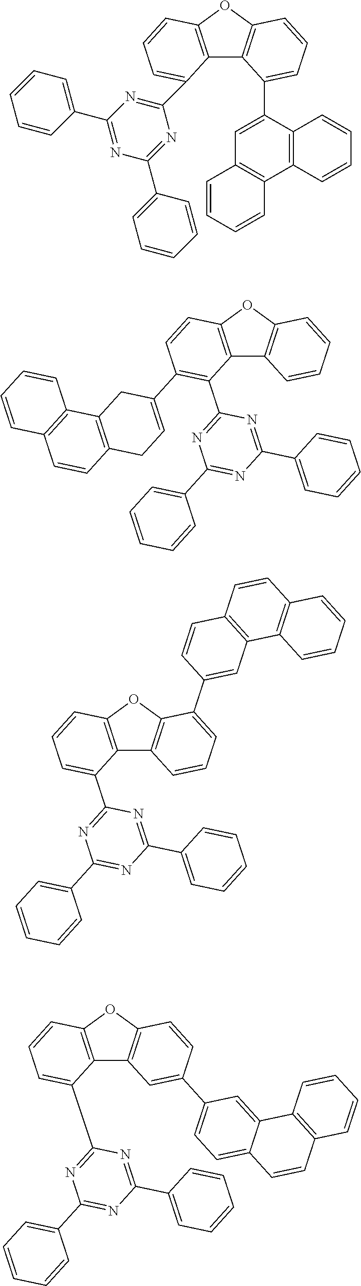 [SMILES: c1ccc(-c2nc(-c3ccccc3)nc(-c3cccc4oc5cccc(-c6cc7ccccc7c7ccccc67)c5c34)n2)cc1 ; C1=C(c2ccc3oc4ccccc4c3c2-c2nc(-c3ccccc3)nc(-c3ccccc3)n2)Cc2c(ccc3ccccc23)C1 ; c1ccc(-c2nc(-c3ccccc3)nc(-c3cccc4oc5c(-c6ccc7ccc8ccccc8c7c6)cccc5c34)n2)cc1 ; c1ccc(-c2nc(-c3ccccc3)nc(-c3cccc4oc5ccc(-c6ccc7ccc8ccccc8c7c6)cc5c34)n2)cc1]

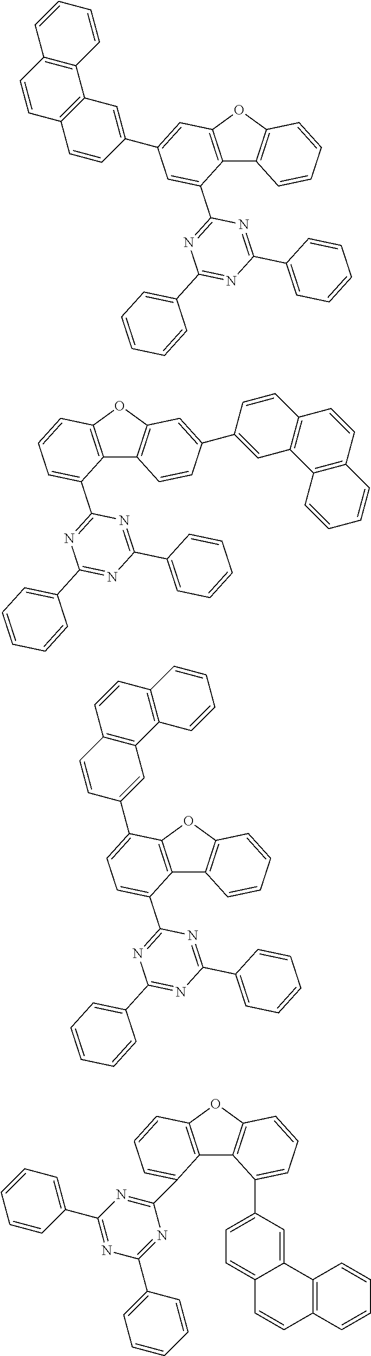 [SMILES: c1ccc(-c2nc(-c3ccccc3)nc(-c3cc(-c4ccc5ccc6ccccc6c5c4)cc4oc5ccccc5c34)n2)cc1 ; c1ccc(-c2nc(-c3ccccc3)nc(-c3cccc4oc5cc(-c6ccc7ccc8ccccc8c7c6)ccc5c34)n2)cc1 ; c1ccc(-c2nc(-c3ccccc3)nc(-c3ccc(-c4ccc5ccc6ccccc6c5c4)c4oc5ccccc5c34)n2)cc1 ; c1ccc(-c2nc(-c3ccccc3)nc(-c3cccc4oc5cccc(-c6ccc7ccc8ccccc8c7c6)c5c34)n2)cc1]

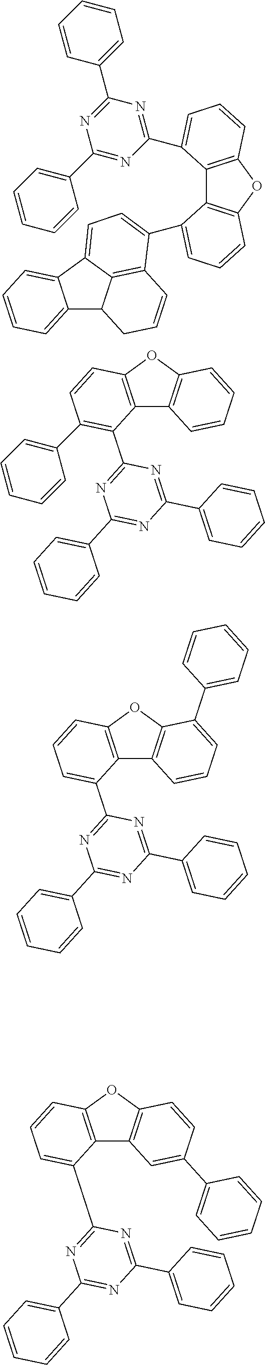

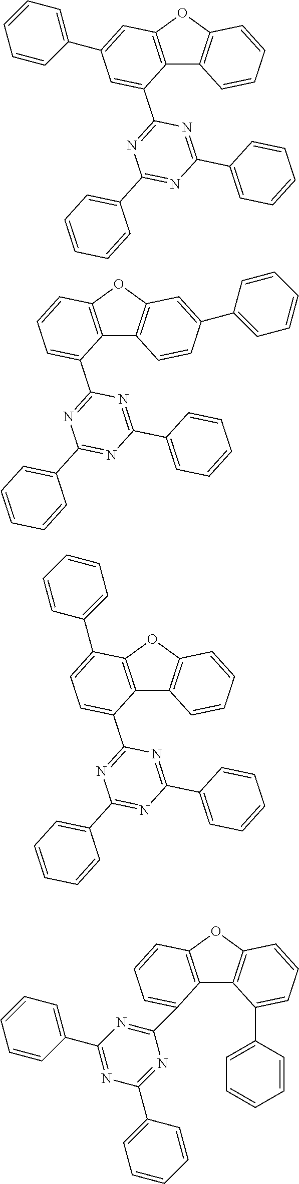

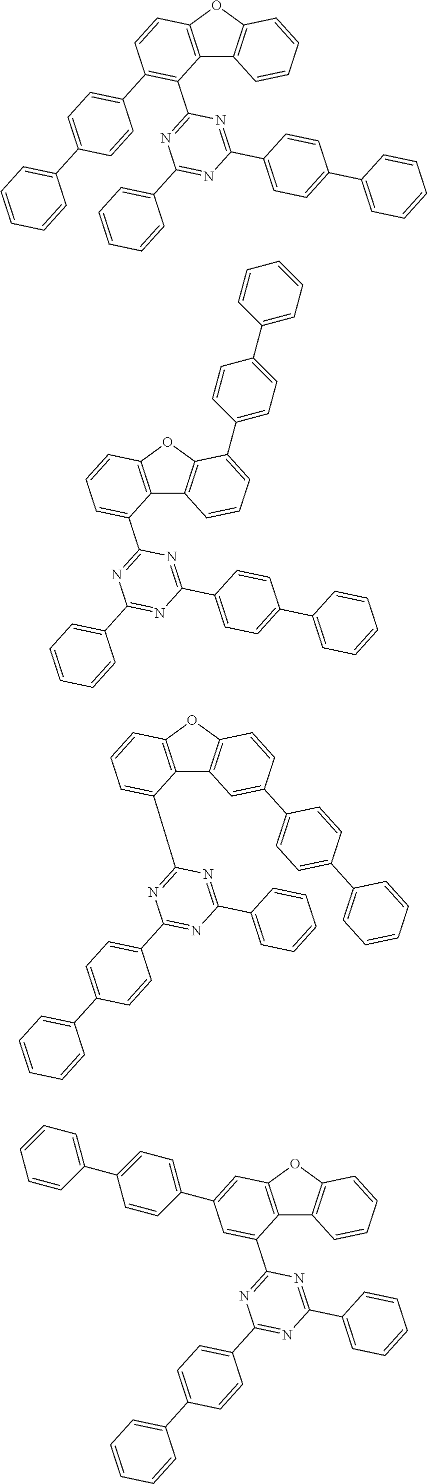 [SMILES: c1ccc(-c2ccc(-c3nc(-c4ccccc4)nc(-c4c(-c5ccc(-c6ccccc6)cc5)ccc5oc6ccccc6c45)n3)cc2)cc1 ; c1ccc(-c2ccc(-c3nc(-c4ccccc4)nc(-c4cccc5oc6c(-c7ccc(-c8ccccc8)cc7)cccc6c45)n3)cc2)cc1 ; c1ccc(-c2ccc(-c3ccc4oc5cccc(-c6nc(-c7ccccc7)nc(-c7ccc(-c8ccccc8)cc7)n6)c5c4c3)cc2)cc1 ; c1ccc(-c2ccc(-c3cc(-c4nc(-c5ccccc5)nc(-c5ccc(-c6ccccc6)cc5)n4)c4c(c3)oc3ccccc34)cc2)cc1]

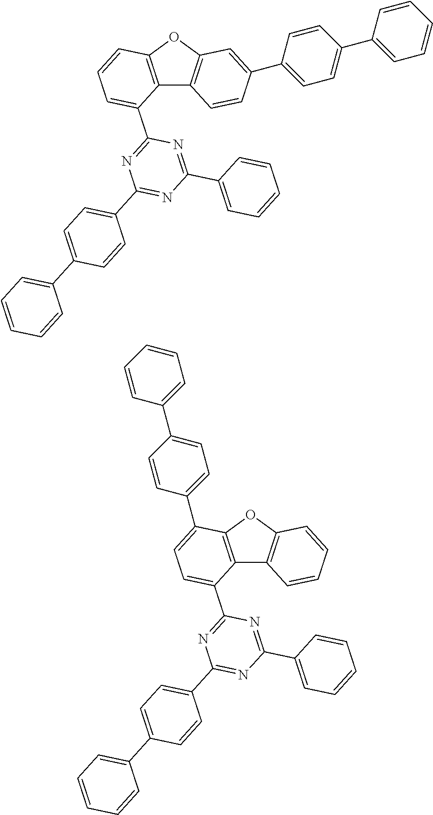 [SMILES: c1ccc(-c2ccc(-c3ccc4c(c3)oc3cccc(-c5nc(-c6ccccc6)nc(-c6ccc(-c7ccccc7)cc6)n5)c34)cc2)cc1 ; c1ccc(-c2ccc(-c3nc(-c4ccccc4)nc(-c4ccc(-c5ccc(-c6ccccc6)cc5)c5oc6ccccc6c45)n3)cc2)cc1]

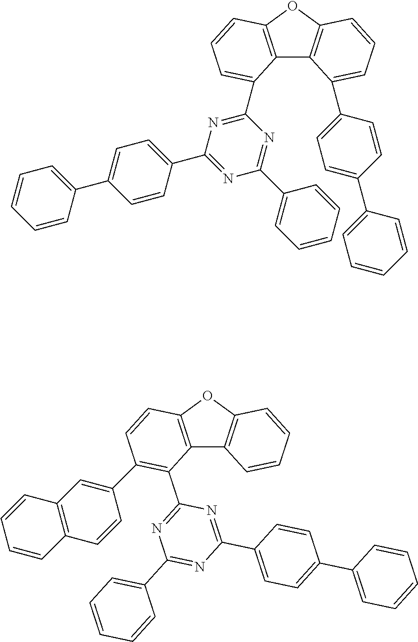 [SMILES: c1ccc(-c2ccc(-c3nc(-c4ccccc4)nc(-c4cccc5oc6cccc(-c7ccc(-c8ccccc8)cc7)c6c45)n3)cc2)cc1 ; c1ccc(-c2ccc(-c3nc(-c4ccccc4)nc(-c4c(-c5ccc6ccccc6c5)ccc5oc6ccccc6c45)n3)cc2)cc1]

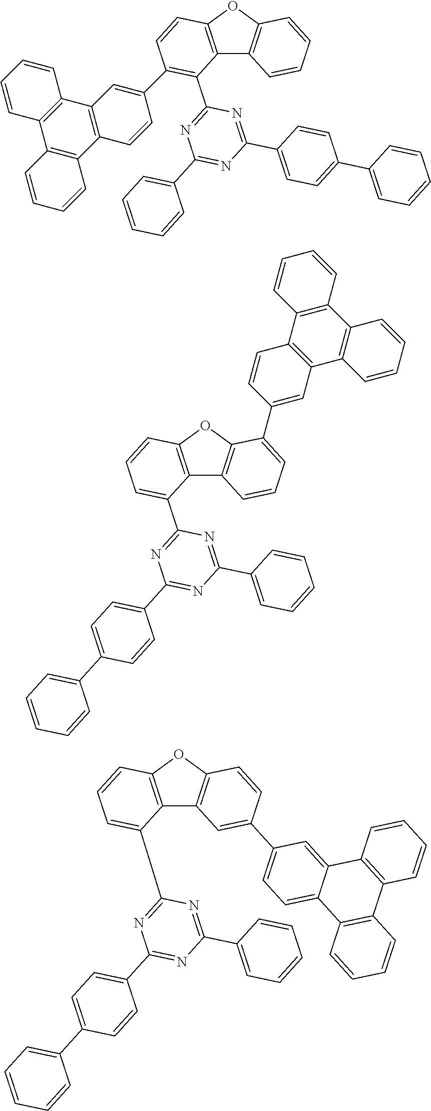 [SMILES: c1ccc(-c2ccc(-c3nc(-c4ccccc4)nc(-c4c(-c5ccc6c7ccccc7c7ccccc7c6c5)ccc5oc6ccccc6c45)n3)cc2)cc1 ; c1ccc(-c2ccc(-c3nc(-c4ccccc4)nc(-c4cccc5oc6c(-c7ccc8c9ccccc9c9ccccc9c8c7)cccc6c45)n3)cc2)cc1 ; c1ccc(-c2ccc(-c3nc(-c4ccccc4)nc(-c4cccc5oc6ccc(-c7ccc8c9ccccc9c9ccccc9c8c7)cc6c45)n3)cc2)cc1]

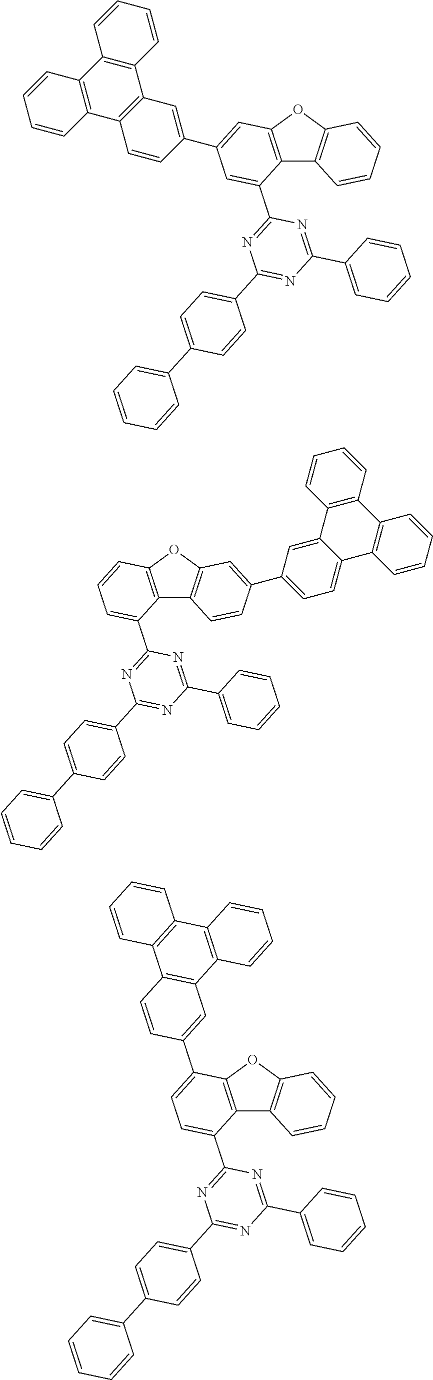 [SMILES: c1ccc(-c2ccc(-c3nc(-c4ccccc4)nc(-c4cc(-c5ccc6c7ccccc7c7ccccc7c6c5)cc5oc6ccccc6c45)n3)cc2)cc1 ; c1ccc(-c2ccc(-c3nc(-c4ccccc4)nc(-c4cccc5oc6cc(-c7ccc8c9ccccc9c9ccccc9c8c7)ccc6c45)n3)cc2)cc1 ; c1ccc(-c2ccc(-c3nc(-c4ccccc4)nc(-c4ccc(-c5ccc6c7ccccc7c7ccccc7c6c5)c5oc6ccccc6c45)n3)cc2)cc1]

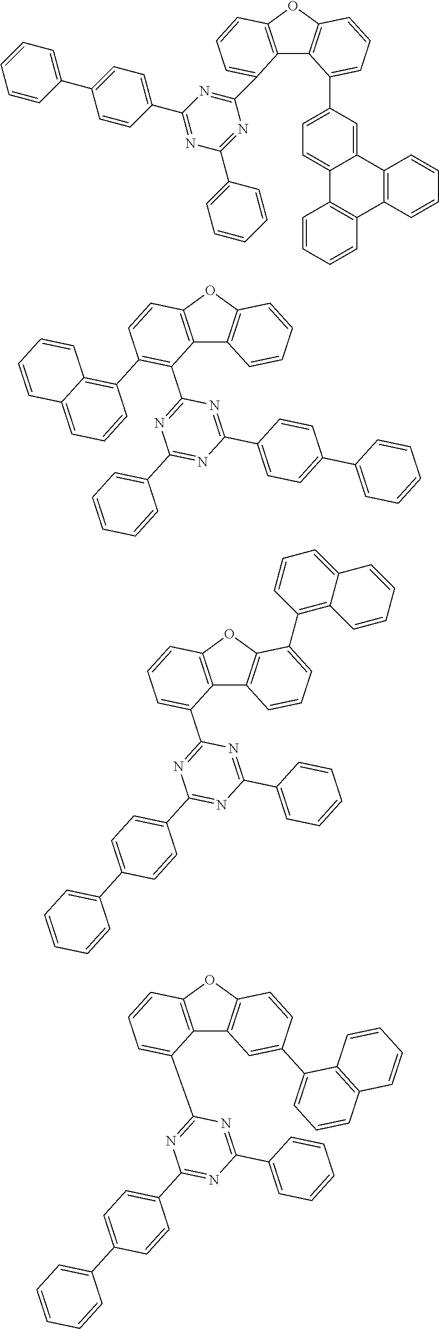 [SMILES: c1ccc(-c2ccc(-c3nc(-c4ccccc4)nc(-c4cccc5oc6cccc(-c7ccc8c9ccccc9c9ccccc9c8c7)c6c45)n3)cc2)cc1 ; c1ccc(-c2ccc(-c3nc(-c4ccccc4)nc(-c4c(-c5cccc6ccccc56)ccc5oc6ccccc6c45)n3)cc2)cc1 ; c1ccc(-c2ccc(-c3nc(-c4ccccc4)nc(-c4cccc5oc6c(-c7cccc8ccccc78)cccc6c45)n3)cc2)cc1 ; c1ccc(-c2ccc(-c3nc(-c4ccccc4)nc(-c4cccc5oc6ccc(-c7cccc8ccccc78)cc6c45)n3)cc2)cc1]

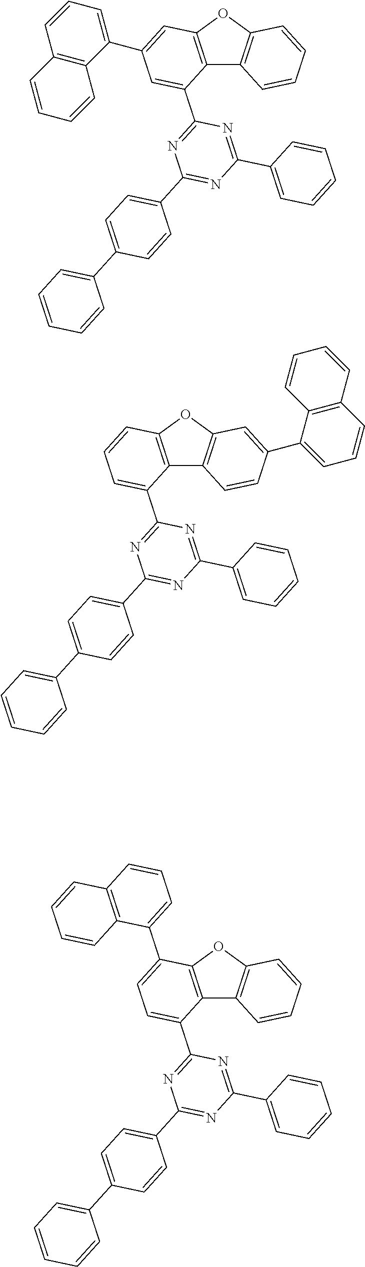 [SMILES: c1ccc(-c2ccc(-c3nc(-c4ccccc4)nc(-c4cc(-c5cccc6ccccc56)cc5oc6ccccc6c45)n3)cc2)cc1 ; c1ccc(-c2ccc(-c3nc(-c4ccccc4)nc(-c4cccc5oc6cc(-c7cccc8ccccc78)ccc6c45)n3)cc2)cc1 ; c1ccc(-c2ccc(-c3nc(-c4ccccc4)nc(-c4ccc(-c5cccc6ccccc56)c5oc6ccccc6c45)n3)cc2)cc1]

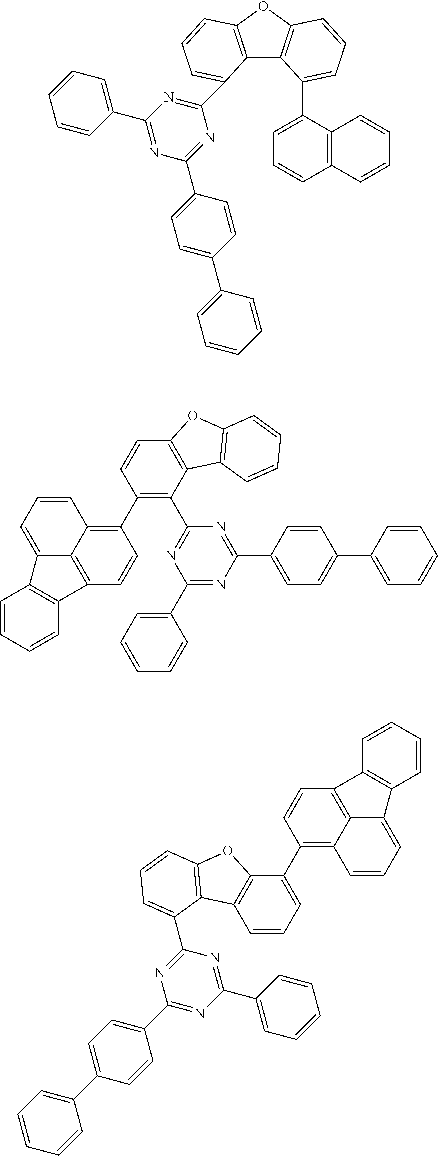 [SMILES: c1ccc(-c2ccc(-c3nc(-c4ccccc4)nc(-c4cccc5oc6cccc(-c7cccc8ccccc78)c6c45)n3)cc2)cc1 ; c1ccc(-c2ccc(-c3nc(-c4ccccc4)nc(-c4c(-c5ccc6c7c(cccc57)-c5ccccc5-6)ccc5oc6ccccc6c45)n3)cc2)cc1 ; c1ccc(-c2ccc(-c3nc(-c4ccccc4)nc(-c4cccc5oc6c(-c7ccc8c9c(cccc79)-c7ccccc7-8)cccc6c45)n3)cc2)cc1]

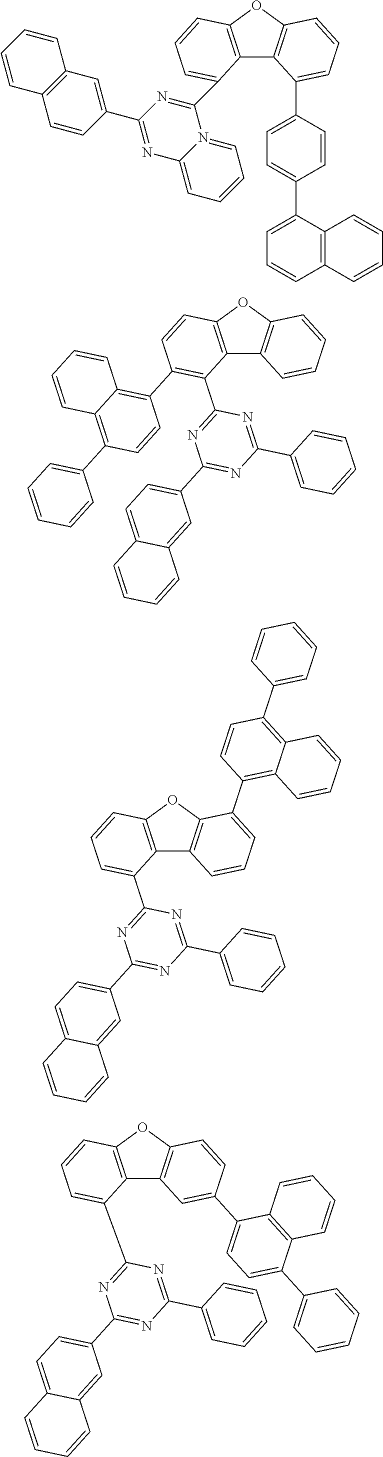

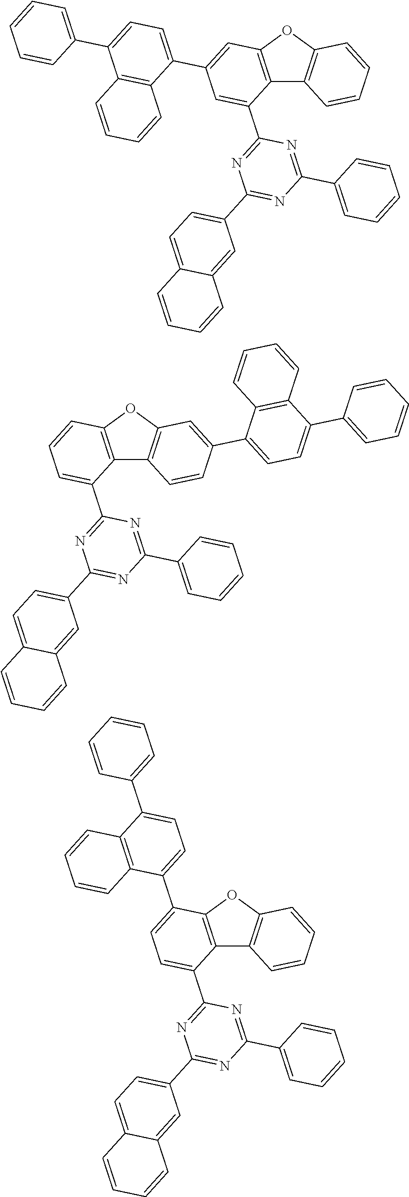 [SMILES: c1ccc(-c2nc(-c3ccc4ccccc4c3)nc(-c3cc(-c4ccc(-c5ccccc5)c5ccccc45)cc4oc5ccccc5c34)n2)cc1 ; c1ccc(-c2nc(-c3ccc4ccccc4c3)nc(-c3cccc4oc5cc(-c6ccc(-c7ccccc7)c7ccccc67)ccc5c34)n2)cc1 ; c1ccc(-c2nc(-c3ccc4ccccc4c3)nc(-c3ccc(-c4ccc(-c5ccccc5)c5ccccc45)c4oc5ccccc5c34)n2)cc1]

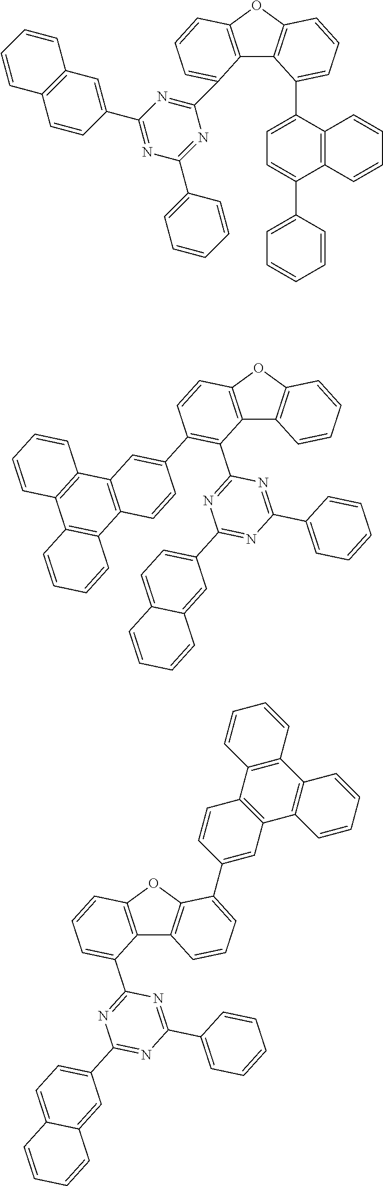 [SMILES: c1ccc(-c2nc(-c3ccc4ccccc4c3)nc(-c3cccc4oc5cccc(-c6ccc(-c7ccccc7)c7ccccc67)c5c34)n2)cc1 ; c1ccc(-c2nc(-c3ccc4ccccc4c3)nc(-c3c(-c4ccc5c6ccccc6c6ccccc6c5c4)ccc4oc5ccccc5c34)n2)cc1 ; c1ccc(-c2nc(-c3ccc4ccccc4c3)nc(-c3cccc4oc5c(-c6ccc7c8ccccc8c8ccccc8c7c6)cccc5c34)n2)cc1]

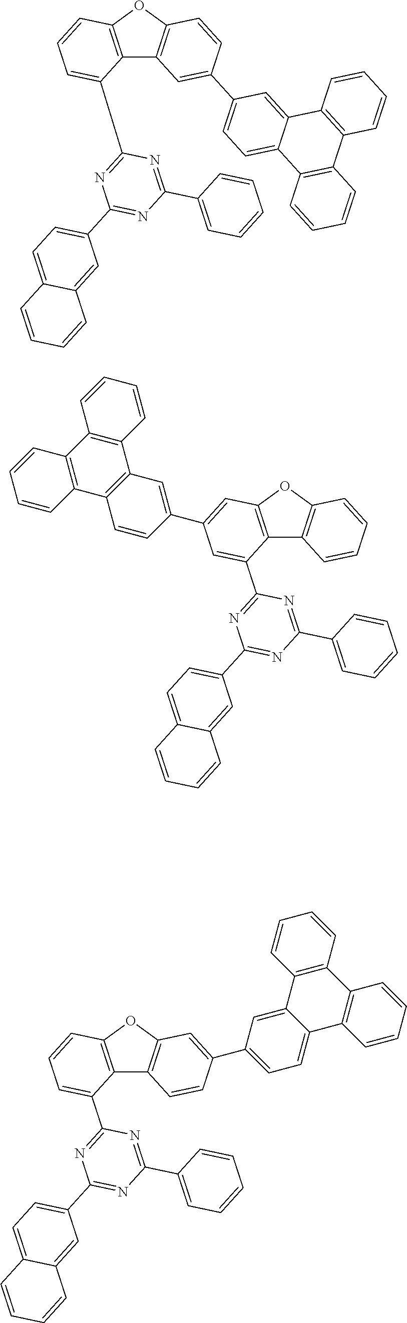 [SMILES: c1ccc(-c2nc(-c3ccc4ccccc4c3)nc(-c3cccc4oc5ccc(-c6ccc7c8ccccc8c8ccccc8c7c6)cc5c34)n2)cc1 ; c1ccc(-c2nc(-c3ccc4ccccc4c3)nc(-c3cc(-c4ccc5c6ccccc6c6ccccc6c5c4)cc4oc5ccccc5c34)n2)cc1 ; c1ccc(-c2nc(-c3ccc4ccccc4c3)nc(-c3cccc4oc5cc(-c6ccc7c8ccccc8c8ccccc8c7c6)ccc5c34)n2)cc1]

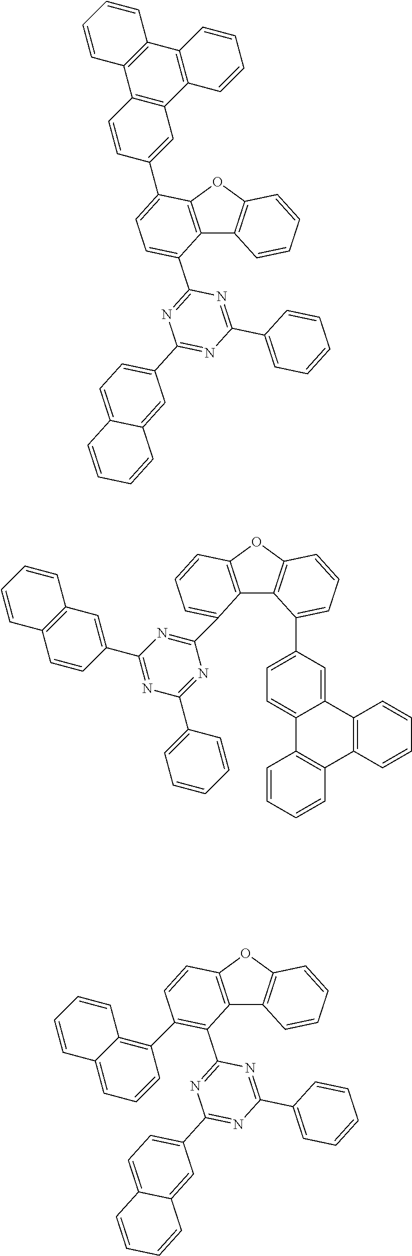 [SMILES: c1ccc(-c2nc(-c3ccc4ccccc4c3)nc(-c3ccc(-c4ccc5c6ccccc6c6ccccc6c5c4)c4oc5ccccc5c34)n2)cc1 ; c1ccc(-c2nc(-c3ccc4ccccc4c3)nc(-c3cccc4oc5cccc(-c6ccc7c8ccccc8c8ccccc8c7c6)c5c34)n2)cc1 ; c1ccc(-c2nc(-c3ccc4ccccc4c3)nc(-c3c(-c4cccc5ccccc45)ccc4oc5ccccc5c34)n2)cc1]

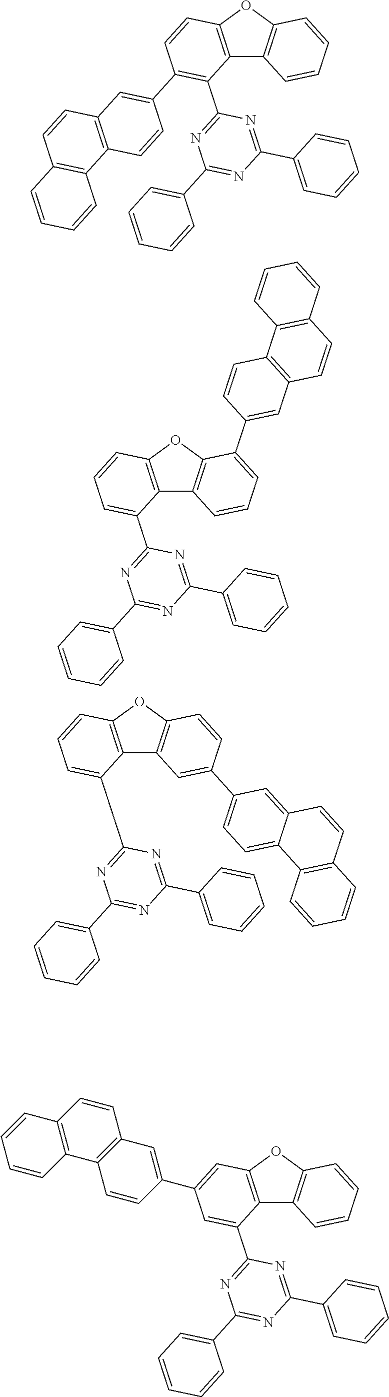

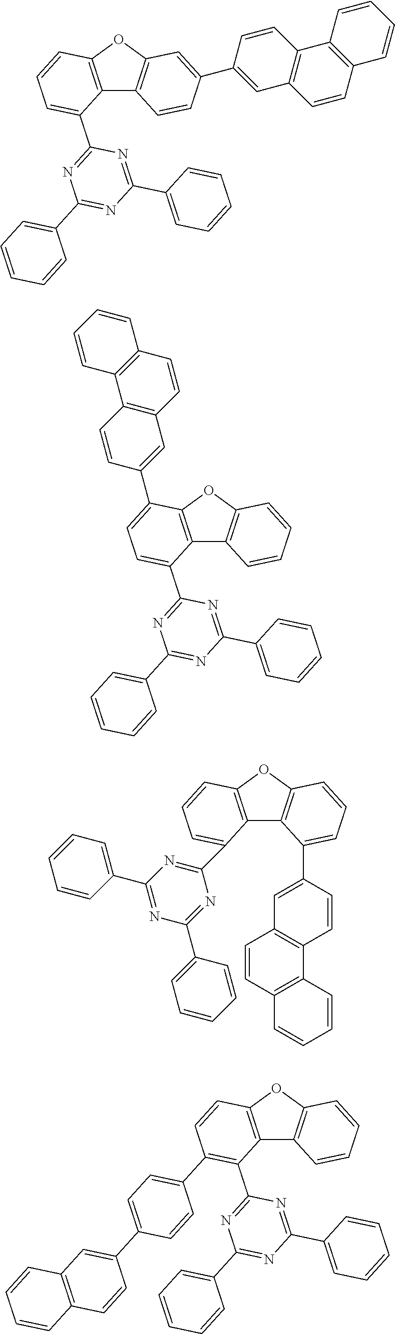 [SMILES: c1ccc(-c2nc(-c3ccccc3)nc(-c3cccc4oc5cc(-c6ccc7c(ccc8ccccc87)c6)ccc5c34)n2)cc1 ; c1ccc(-c2nc(-c3ccccc3)nc(-c3ccc(-c4ccc5c(ccc6ccccc65)c4)c4oc5ccccc5c34)n2)cc1 ; c1ccc(-c2nc(-c3ccccc3)nc(-c3cccc4oc5cccc(-c6ccc7c(ccc8ccccc87)c6)c5c34)n2)cc1 ; c1ccc(-c2nc(-c3ccccc3)nc(-c3c(-c4ccc(-c5ccc6ccccc6c5)cc4)ccc4oc5ccccc5c34)n2)cc1]

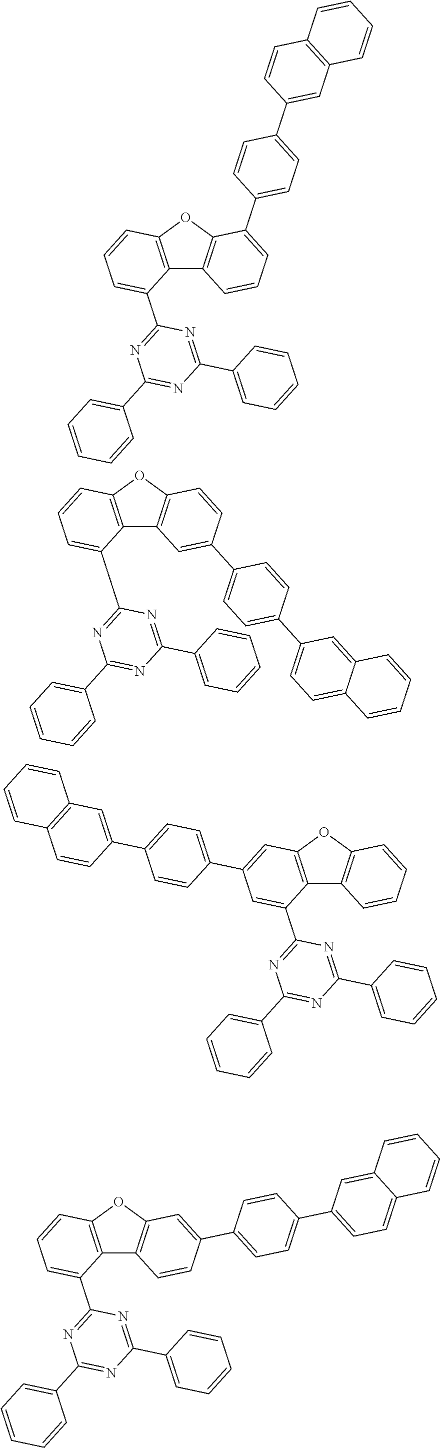

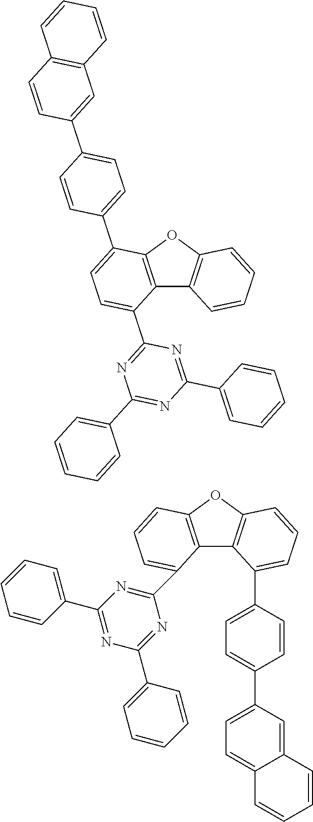 [SMILES: c1ccc(-c2nc(-c3ccccc3)nc(-c3ccc(-c4ccc(-c5ccc6ccccc6c5)cc4)c4oc5ccccc5c34)n2)cc1 ; c1ccc(-c2nc(-c3ccccc3)nc(-c3cccc4oc5cccc(-c6ccc(-c7ccc8ccccc8c7)cc6)c5c34)n2)cc1]

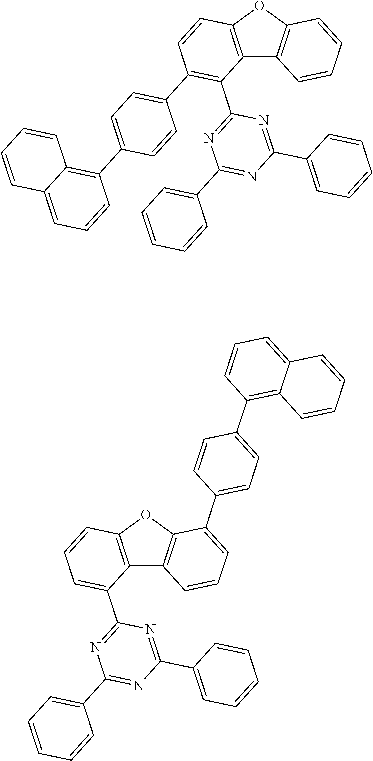 [SMILES: c1ccc(-c2nc(-c3ccccc3)nc(-c3c(-c4ccc(-c5cccc6ccccc56)cc4)ccc4oc5ccccc5c34)n2)cc1 ; c1ccc(-c2nc(-c3ccccc3)nc(-c3cccc4oc5c(-c6ccc(-c7cccc8ccccc78)cc6)cccc5c34)n2)cc1]

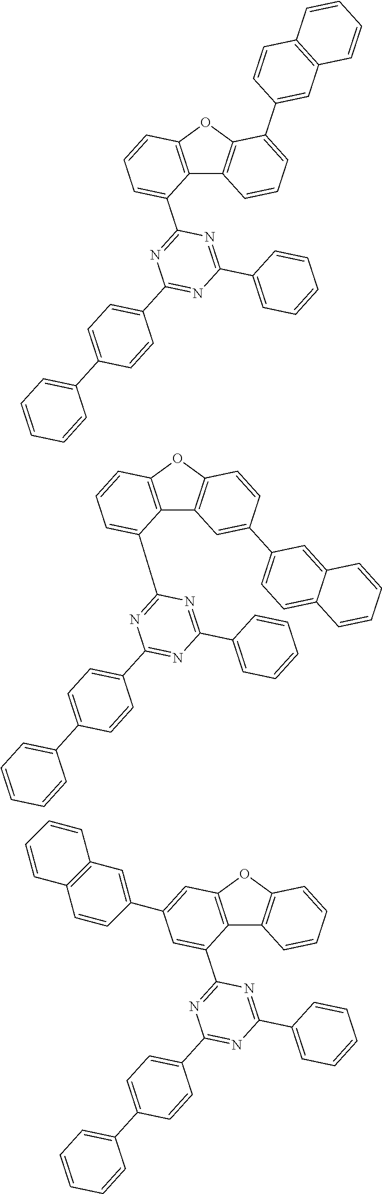

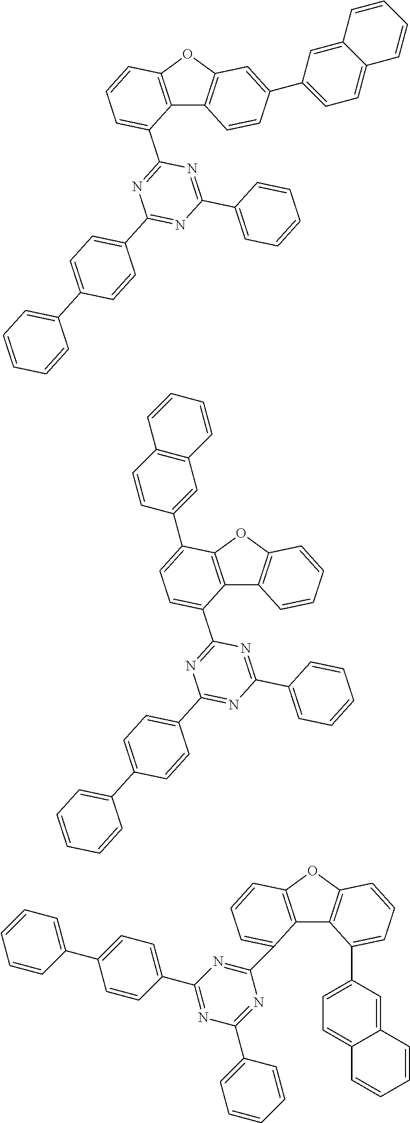 [SMILES: c1ccc(-c2ccc(-c3nc(-c4ccccc4)nc(-c4cccc5oc6cc(-c7ccc8ccccc8c7)ccc6c45)n3)cc2)cc1 ; c1ccc(-c2ccc(-c3nc(-c4ccccc4)nc(-c4ccc(-c5ccc6ccccc6c5)c5oc6ccccc6c45)n3)cc2)cc1 ; c1ccc(-c2ccc(-c3nc(-c4ccccc4)nc(-c4cccc5oc6cccc(-c7ccc8ccccc8c7)c6c45)n3)cc2)cc1]

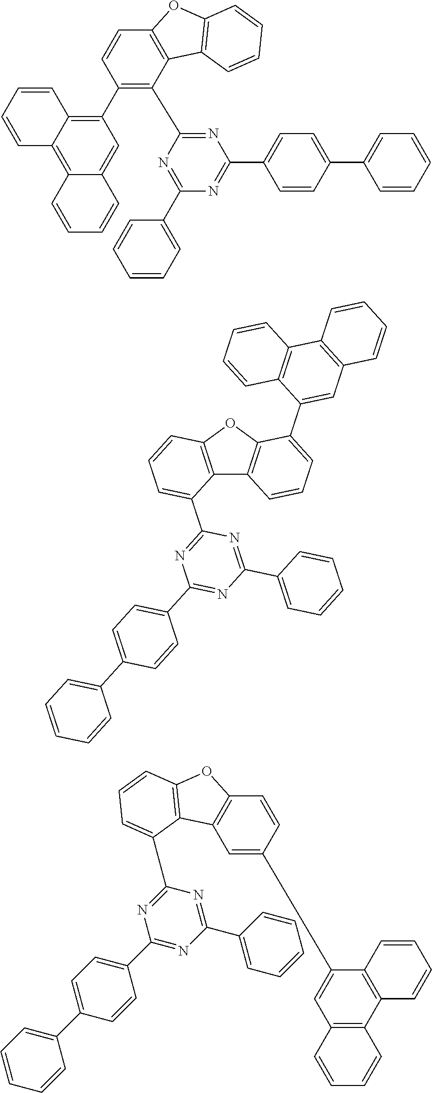 [SMILES: c1ccc(-c2ccc(-c3nc(-c4ccccc4)nc(-c4c(-c5cc6ccccc6c6ccccc56)ccc5oc6ccccc6c45)n3)cc2)cc1 ; c1ccc(-c2ccc(-c3nc(-c4ccccc4)nc(-c4cccc5oc6c(-c7cc8ccccc8c8ccccc78)cccc6c45)n3)cc2)cc1 ; c1ccc(-c2ccc(-c3nc(-c4ccccc4)nc(-c4cccc5oc6ccc(-c7cc8ccccc8c8ccccc78)cc6c45)n3)cc2)cc1]

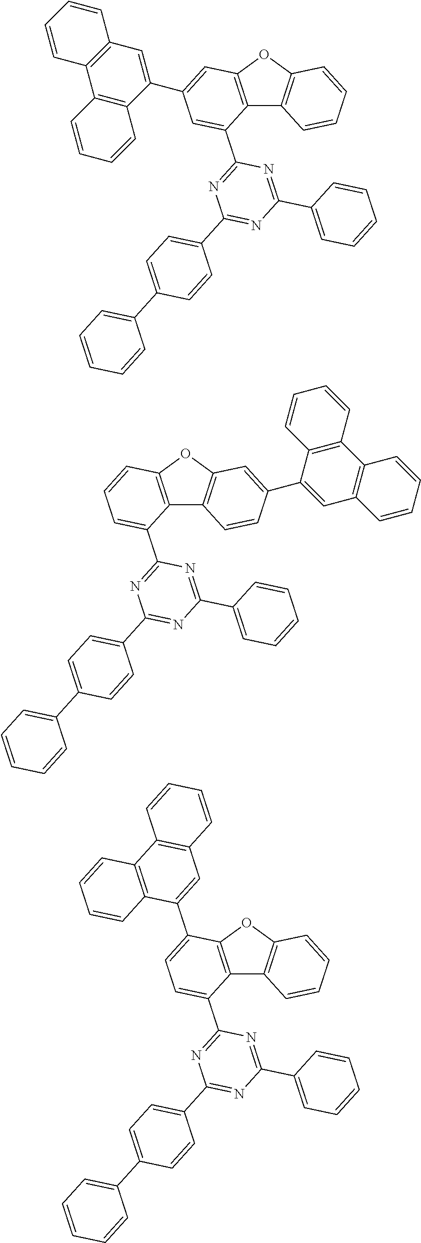 [SMILES: c1ccc(-c2ccc(-c3nc(-c4ccccc4)nc(-c4cc(-c5cc6ccccc6c6ccccc56)cc5oc6ccccc6c45)n3)cc2)cc1 ; c1ccc(-c2ccc(-c3nc(-c4ccccc4)nc(-c4cccc5oc6cc(-c7cc8ccccc8c8ccccc78)ccc6c45)n3)cc2)cc1 ; c1ccc(-c2ccc(-c3nc(-c4ccccc4)nc(-c4ccc(-c5cc6ccccc6c6ccccc56)c5oc6ccccc6c45)n3)cc2)cc1]

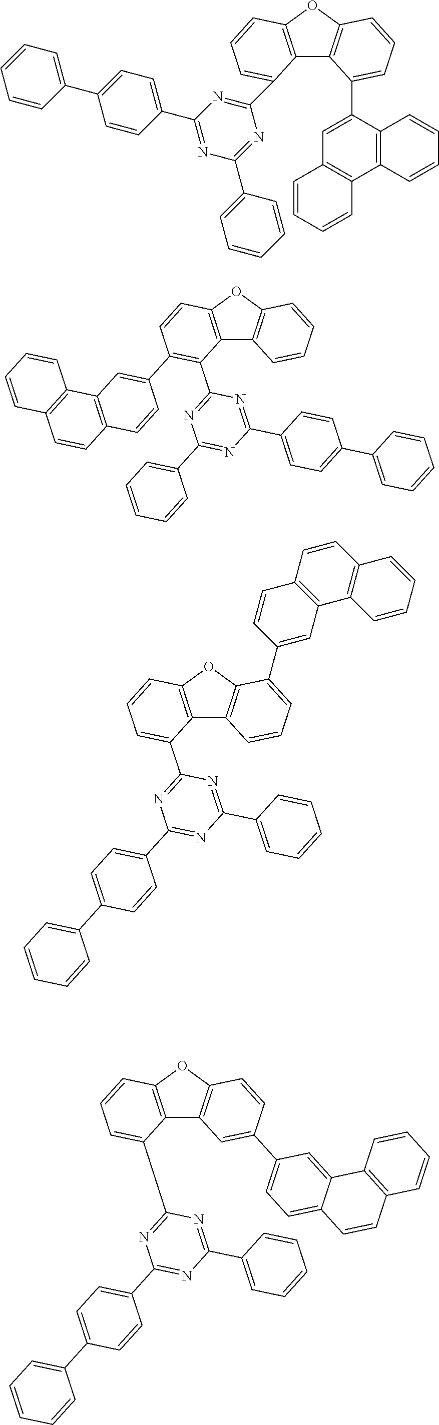

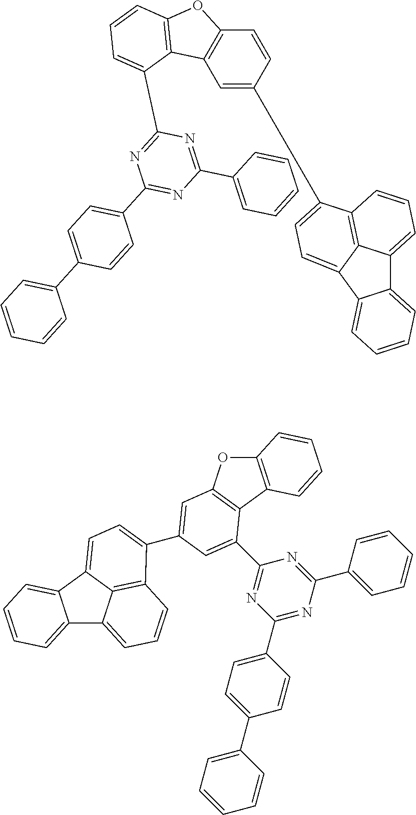 [SMILES: c1ccc(-c2ccc(-c3nc(-c4ccccc4)nc(-c4cccc5oc6ccc(-c7ccc8c9c(cccc79)-c7ccccc7-8)cc6c45)n3)cc2)cc1 ; c1ccc(-c2ccc(-c3nc(-c4ccccc4)nc(-c4cc(-c5ccc6c7c(cccc57)-c5ccccc5-6)cc5oc6ccccc6c45)n3)cc2)cc1]

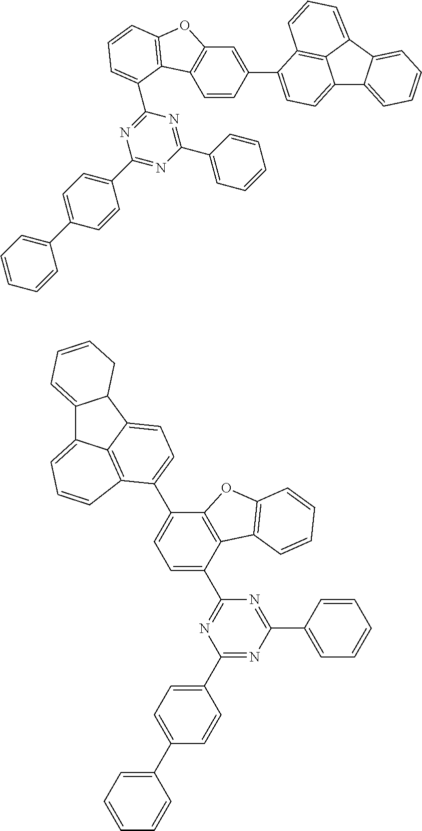 [SMILES: c1ccc(-c2ccc(-c3nc(-c4ccccc4)nc(-c4cccc5oc6cc(-c7ccc8c9c(cccc79)-c7ccccc7-8)ccc6c45)n3)cc2)cc1 ; C1=CCC2C(=C1)c1cccc3c(-c4ccc(-c5nc(-c6ccccc6)nc(-c6ccc(-c7ccccc7)cc6)n5)c5c4oc4ccccc45)ccc2c13]

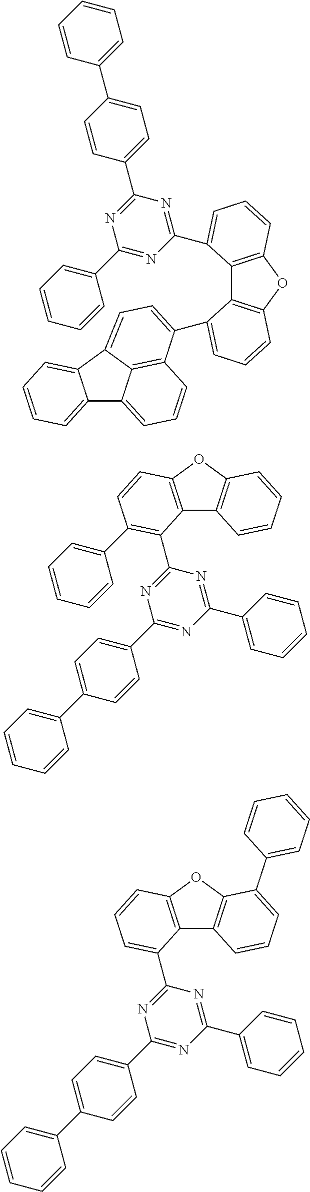

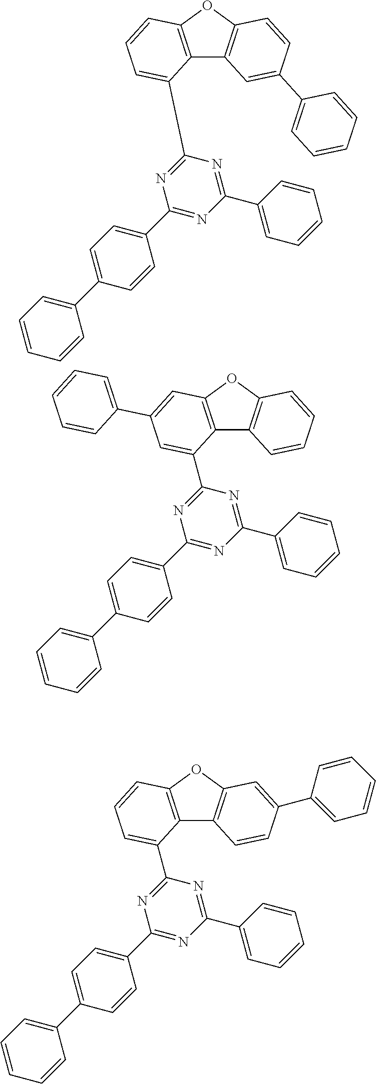

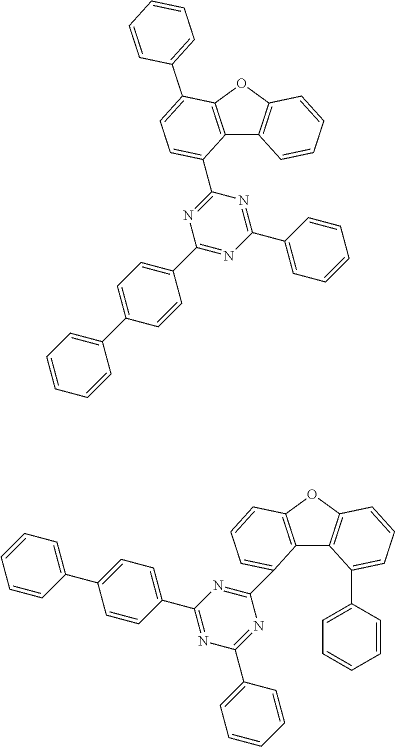 [SMILES: c1ccc(-c2ccc(-c3nc(-c4ccccc4)nc(-c4ccc(-c5ccccc5)c5oc6ccccc6c45)n3)cc2)cc1 ; c1ccc(-c2ccc(-c3nc(-c4ccccc4)nc(-c4cccc5oc6cccc(-c7ccccc7)c6c45)n3)cc2)cc1]

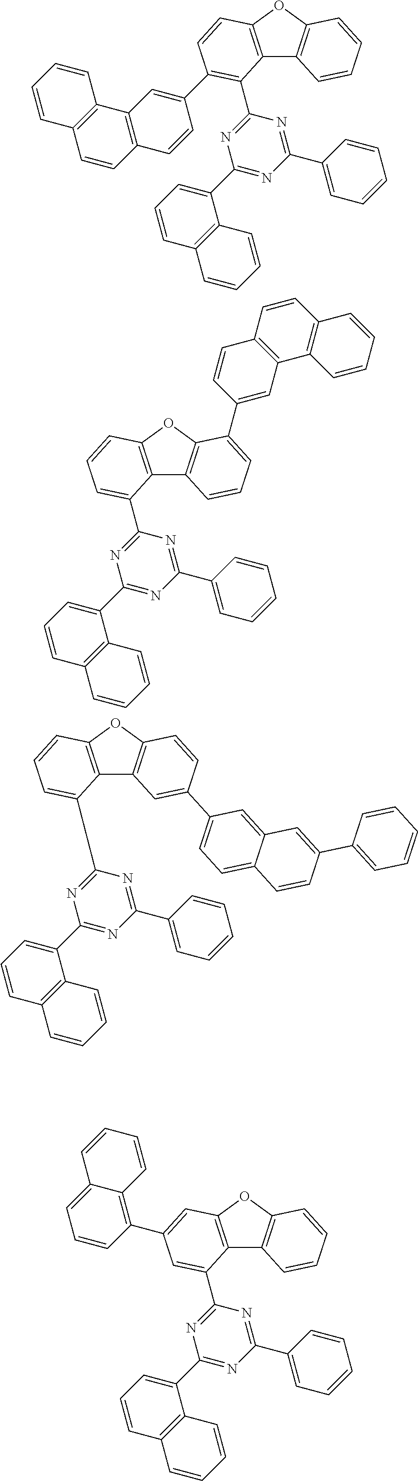

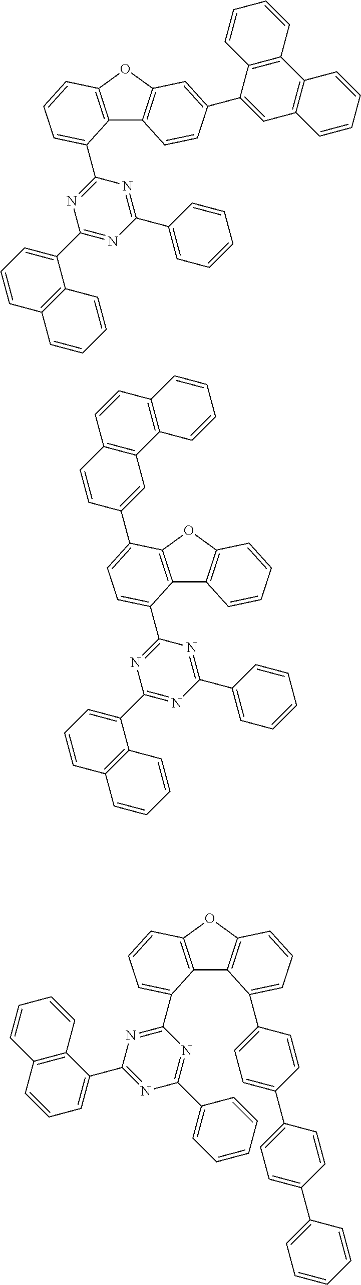 [SMILES: c1ccc(-c2nc(-c3cccc4ccccc34)nc(-c3cccc4oc5cc(-c6cc7ccccc7c7ccccc67)ccc5c34)n2)cc1 ; c1ccc(-c2nc(-c3cccc4ccccc34)nc(-c3ccc(-c4ccc5ccc6ccccc6c5c4)c4oc5ccccc5c34)n2)cc1 ; c1ccc(-c2ccc(-c3ccc(-c4cccc5oc6cccc(-c7nc(-c8ccccc8)nc(-c8cccc9ccccc89)n7)c6c45)cc3)cc2)cc1]

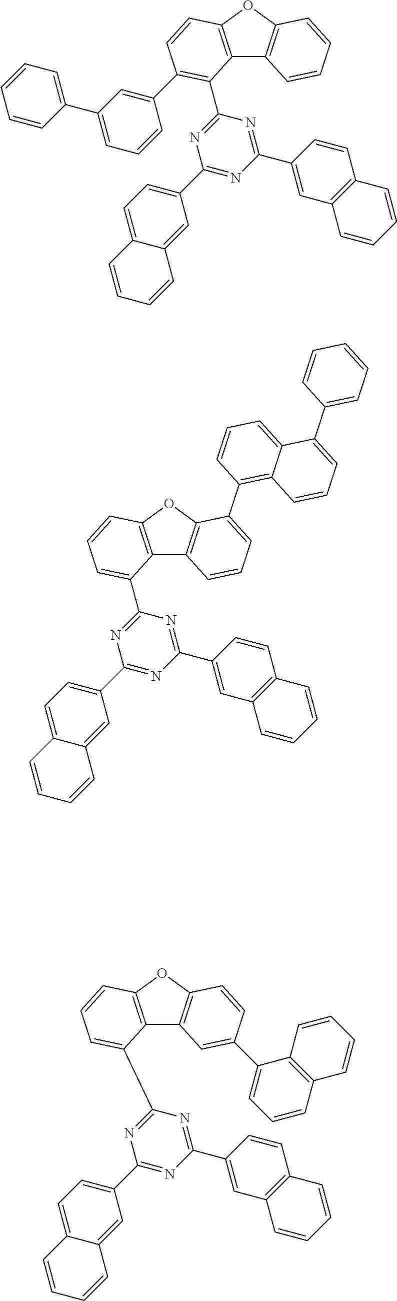 [SMILES: c1ccc(-c2cccc(-c3ccc4oc5ccccc5c4c3-c3nc(-c4ccc5ccccc5c4)nc(-c4ccc5ccccc5c4)n3)c2)cc1 ; c1ccc(-c2cccc3c(-c4cccc5c4oc4cccc(-c6nc(-c7ccc8ccccc8c7)nc(-c7ccc8ccccc8c7)n6)c45)cccc23)cc1 ; c1ccc2cc(-c3nc(-c4ccc5ccccc5c4)nc(-c4cccc5oc6ccc(-c7cccc8ccccc78)cc6c45)n3)ccc2c1]

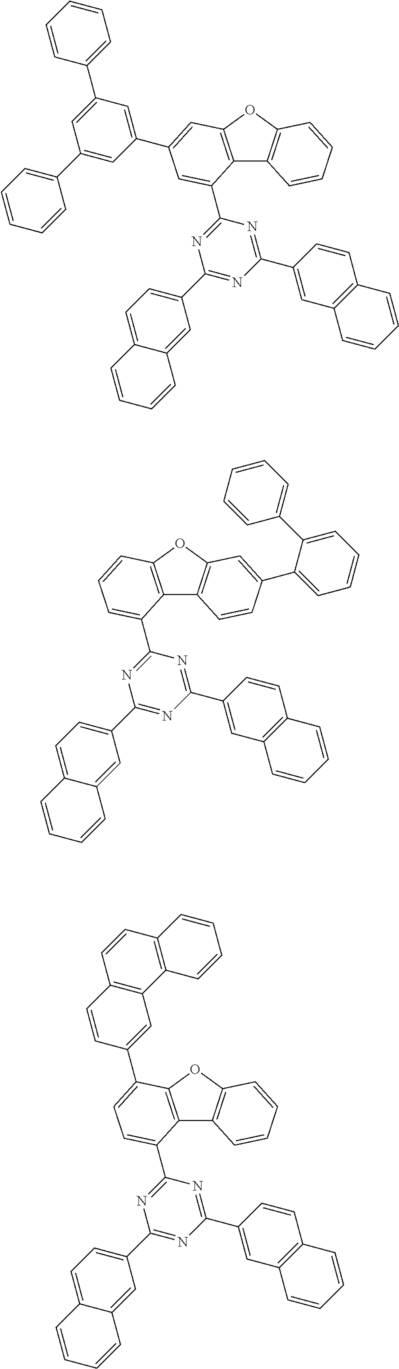 [SMILES: c1ccc(-c2cc(-c3ccccc3)cc(-c3cc(-c4nc(-c5ccc6ccccc6c5)nc(-c5ccc6ccccc6c5)n4)c4c(c3)oc3ccccc34)c2)cc1 ; c1ccc(-c2ccccc2-c2ccc3c(c2)oc2cccc(-c4nc(-c5ccc6ccccc6c5)nc(-c5ccc6ccccc6c5)n4)c23)cc1 ; c1ccc2cc(-c3nc(-c4ccc5ccccc5c4)nc(-c4ccc(-c5ccc6ccc7ccccc7c6c5)c5oc6ccccc6c45)n3)ccc2c1]

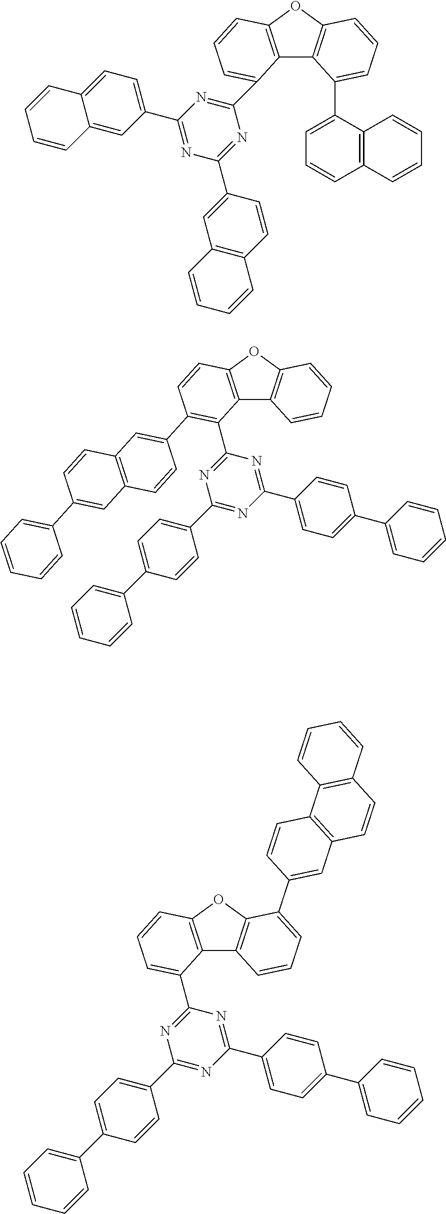

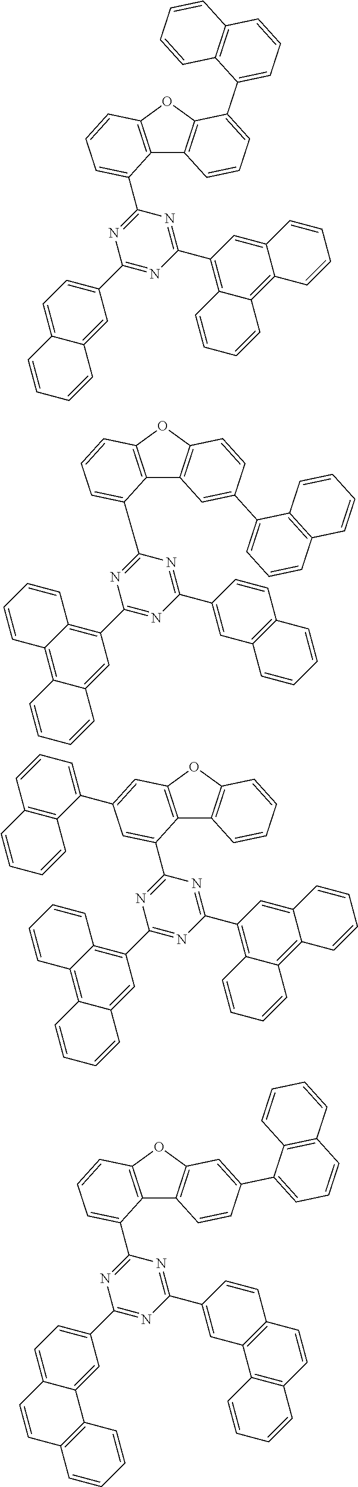

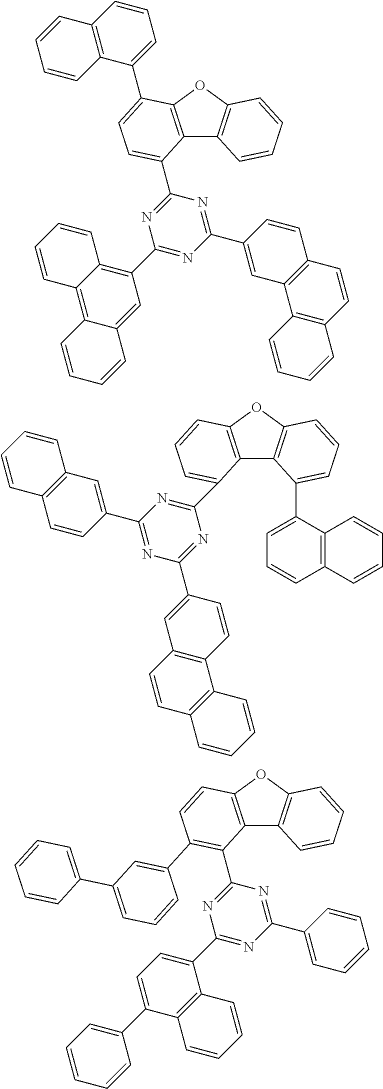

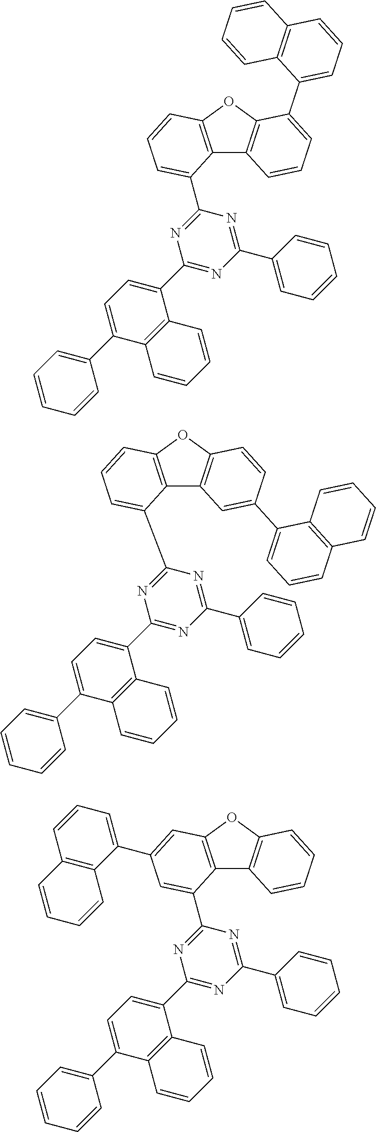 [SMILES: c1ccc(-c2nc(-c3ccc(-c4ccccc4)c4ccccc34)nc(-c3cccc4oc5c(-c6cccc7ccccc67)cccc5c34)n2)cc1 ; c1ccc(-c2nc(-c3ccc(-c4ccccc4)c4ccccc34)nc(-c3cccc4oc5ccc(-c6cccc7ccccc67)cc5c34)n2)cc1 ; c1ccc(-c2nc(-c3ccc(-c4ccccc4)c4ccccc34)nc(-c3cc(-c4cccc5ccccc45)cc4oc5ccccc5c34)n2)cc1]

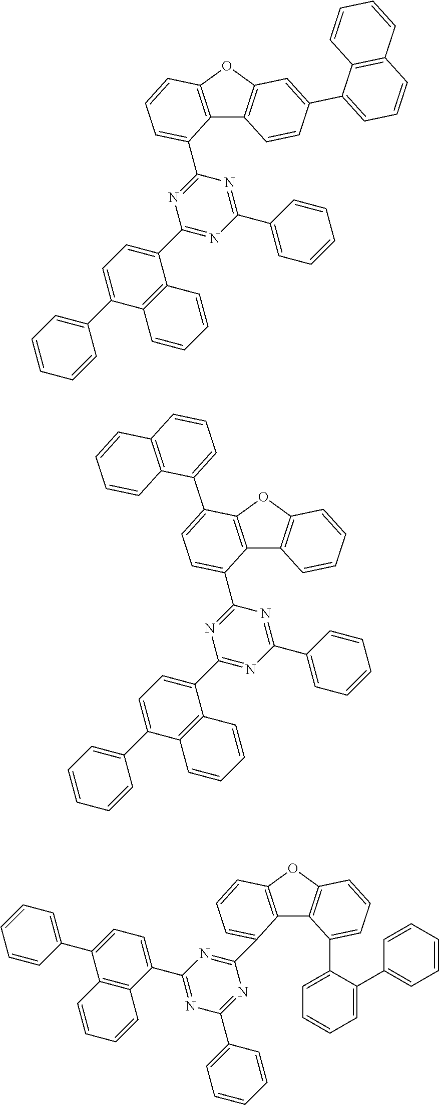 [SMILES: c1ccc(-c2nc(-c3ccc(-c4ccccc4)c4ccccc34)nc(-c3cccc4oc5cc(-c6cccc7ccccc67)ccc5c34)n2)cc1 ; c1ccc(-c2nc(-c3ccc(-c4ccccc4)c4ccccc34)nc(-c3ccc(-c4cccc5ccccc45)c4oc5ccccc5c34)n2)cc1 ; c1ccc(-c2nc(-c3ccc(-c4ccccc4)c4ccccc34)nc(-c3cccc4oc5cccc(-c6ccccc6-c6ccccc6)c5c34)n2)cc1]

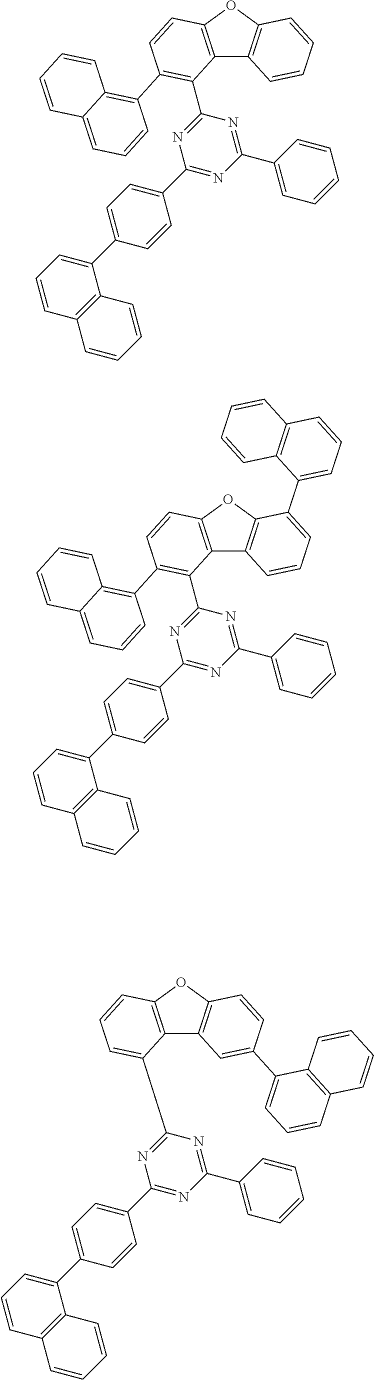 [SMILES: c1ccc(-c2nc(-c3ccc(-c4cccc5ccccc45)cc3)nc(-c3c(-c4cccc5ccccc45)ccc4oc5ccccc5c34)n2)cc1 ; c1ccc(-c2nc(-c3ccc(-c4cccc5ccccc45)cc3)nc(-c3c(-c4cccc5ccccc45)ccc4oc5c(-c6cccc7ccccc67)cccc5c34)n2)cc1 ; c1ccc(-c2nc(-c3ccc(-c4cccc5ccccc45)cc3)nc(-c3cccc4oc5ccc(-c6cccc7ccccc67)cc5c34)n2)cc1]

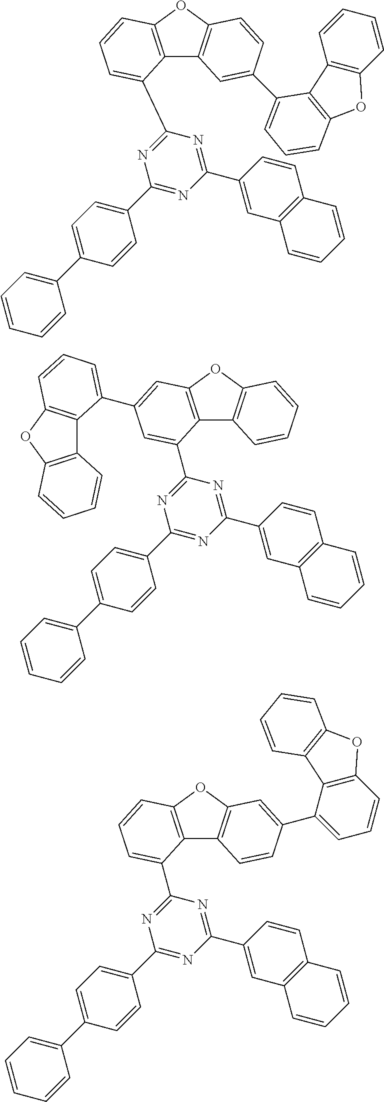

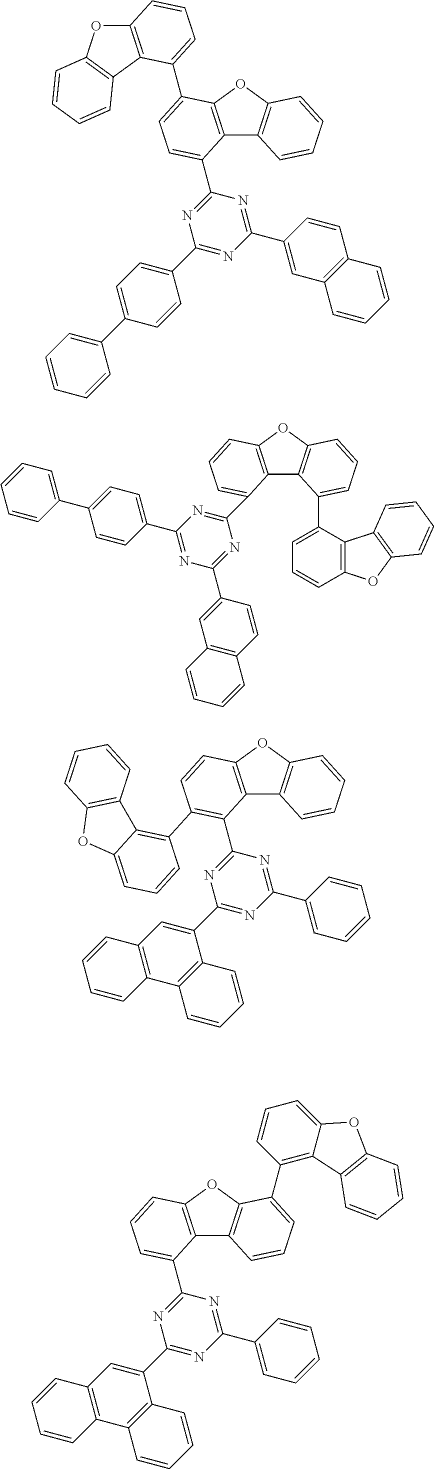 [SMILES: c1ccc(-c2ccc(-c3nc(-c4ccc5ccccc5c4)nc(-c4ccc(-c5cccc6oc7ccccc7c56)c5oc6ccccc6c45)n3)cc2)cc1 ; c1ccc(-c2ccc(-c3nc(-c4ccc5ccccc5c4)nc(-c4cccc5oc6cccc(-c7cccc8oc9ccccc9c78)c6c45)n3)cc2)cc1 ; c1ccc(-c2nc(-c3cc4ccccc4c4ccccc34)nc(-c3c(-c4cccc5oc6ccccc6c45)ccc4oc5ccccc5c34)n2)cc1 ; c1ccc(-c2nc(-c3cc4ccccc4c4ccccc34)nc(-c3cccc4oc5c(-c6cccc7oc8ccccc8c67)cccc5c34)n2)cc1]

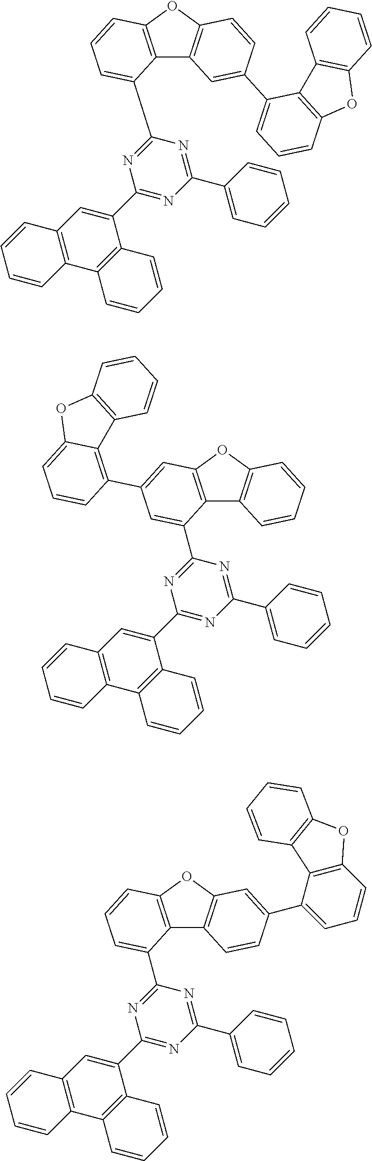

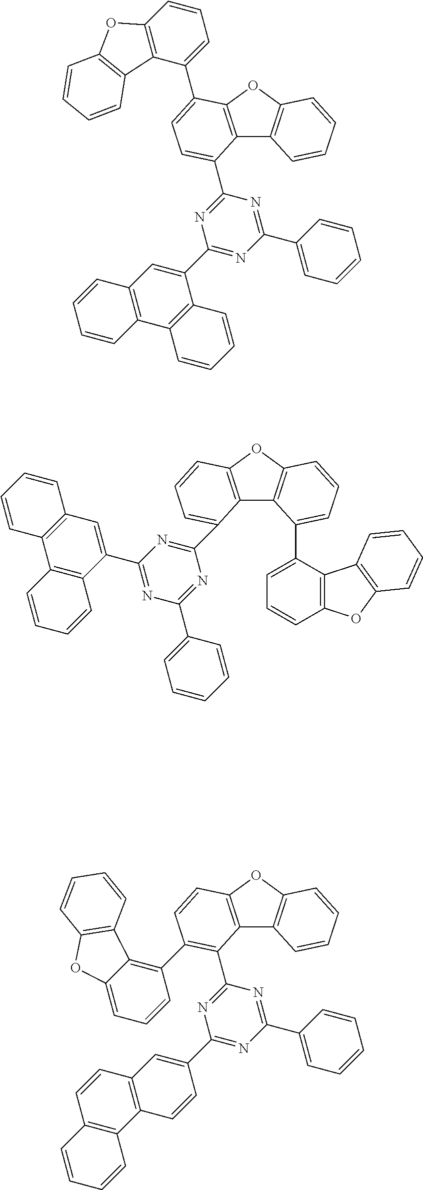 [SMILES: c1ccc(-c2nc(-c3cc4ccccc4c4ccccc34)nc(-c3ccc(-c4cccc5oc6ccccc6c45)c4oc5ccccc5c34)n2)cc1 ; c1ccc(-c2nc(-c3cc4ccccc4c4ccccc34)nc(-c3cccc4oc5cccc(-c6cccc7oc8ccccc8c67)c5c34)n2)cc1 ; c1ccc(-c2nc(-c3ccc4c(ccc5ccccc54)c3)nc(-c3c(-c4cccc5oc6ccccc6c45)ccc4oc5ccccc5c34)n2)cc1]

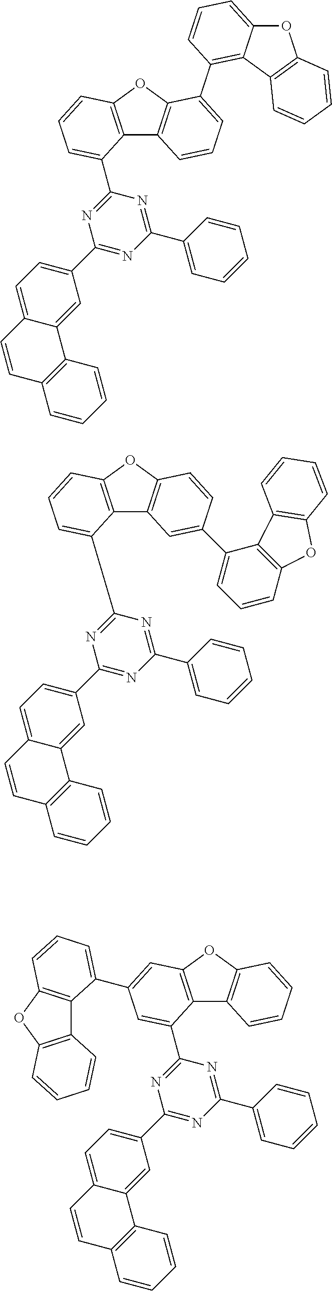 [SMILES: c1ccc(-c2nc(-c3ccc4ccc5ccccc5c4c3)nc(-c3cccc4oc5c(-c6cccc7oc8ccccc8c67)cccc5c34)n2)cc1 ; c1ccc(-c2nc(-c3ccc4ccc5ccccc5c4c3)nc(-c3cccc4oc5ccc(-c6cccc7oc8ccccc8c67)cc5c34)n2)cc1 ; c1ccc(-c2nc(-c3ccc4ccc5ccccc5c4c3)nc(-c3cc(-c4cccc5oc6ccccc6c45)cc4oc5ccccc5c34)n2)cc1]

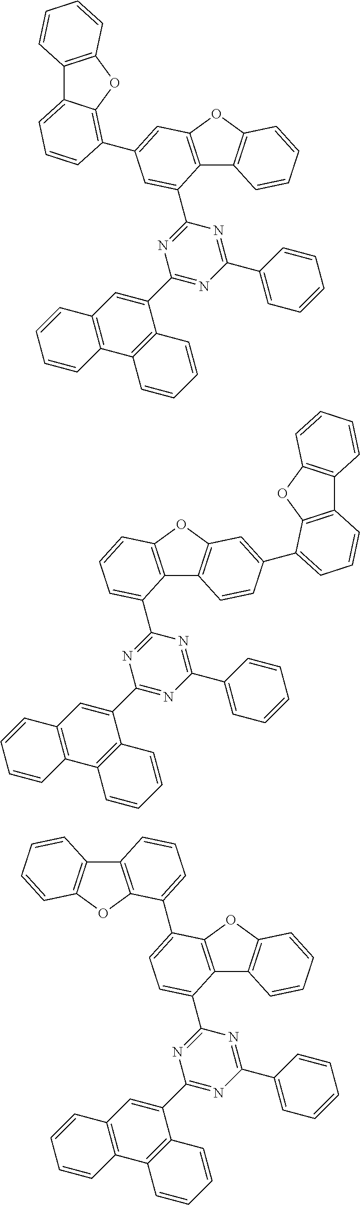

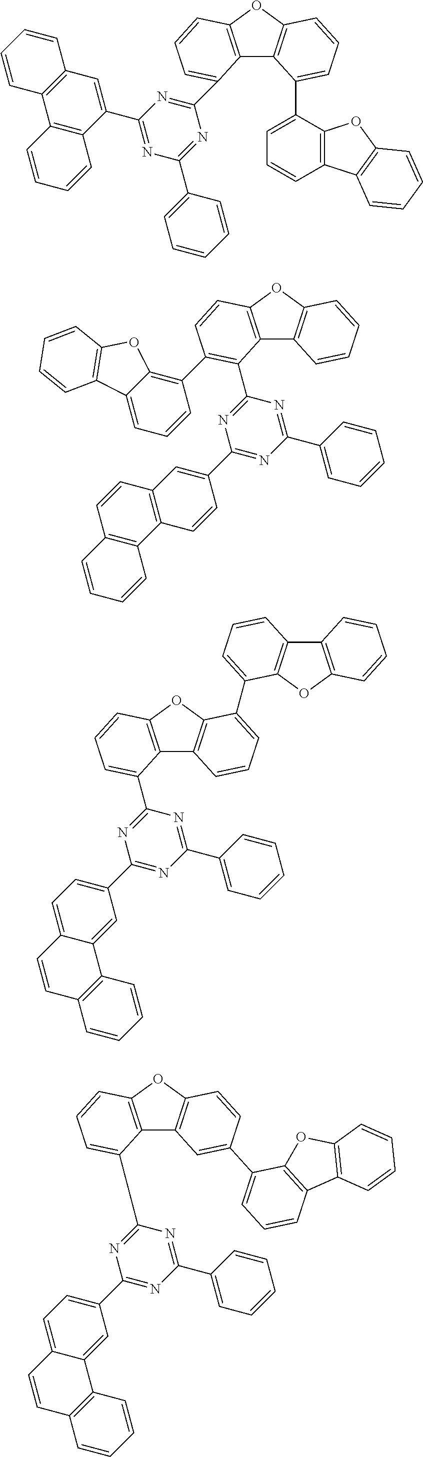

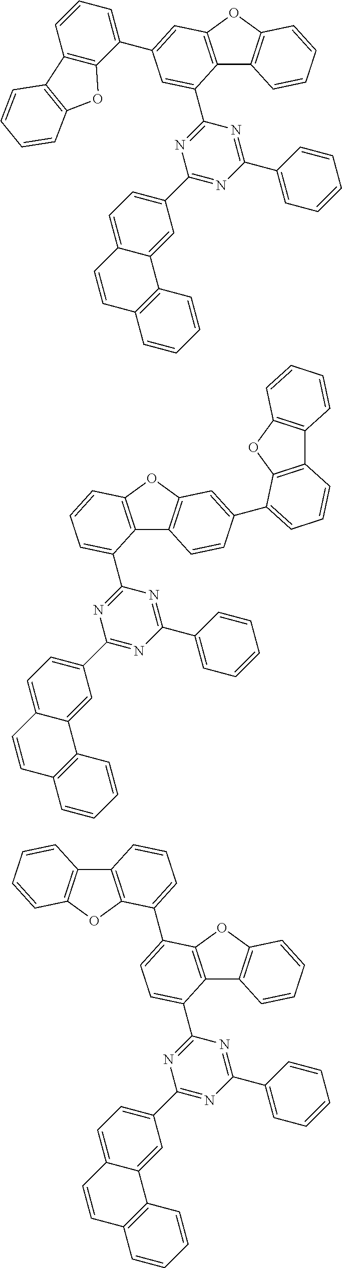

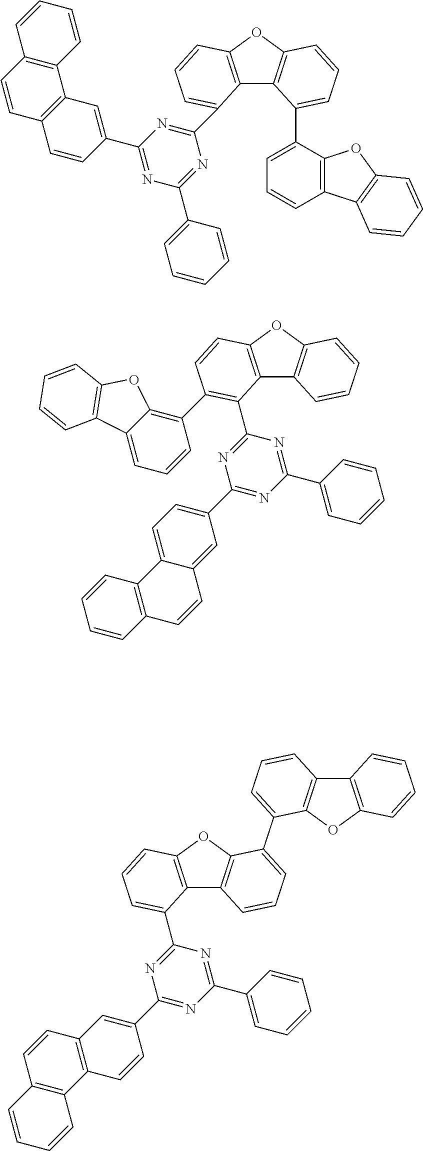 [SMILES: c1ccc(-c2nc(-c3ccc4ccc5ccccc5c4c3)nc(-c3cccc4oc5cccc(-c6cccc7c6oc6ccccc67)c5c34)n2)cc1 ; c1ccc(-c2nc(-c3ccc4c(ccc5ccccc54)c3)nc(-c3c(-c4cccc5c4oc4ccccc45)ccc4oc5ccccc5c34)n2)cc1 ; c1ccc(-c2nc(-c3ccc4c(ccc5ccccc54)c3)nc(-c3cccc4oc5c(-c6cccc7c6oc6ccccc67)cccc5c34)n2)cc1]

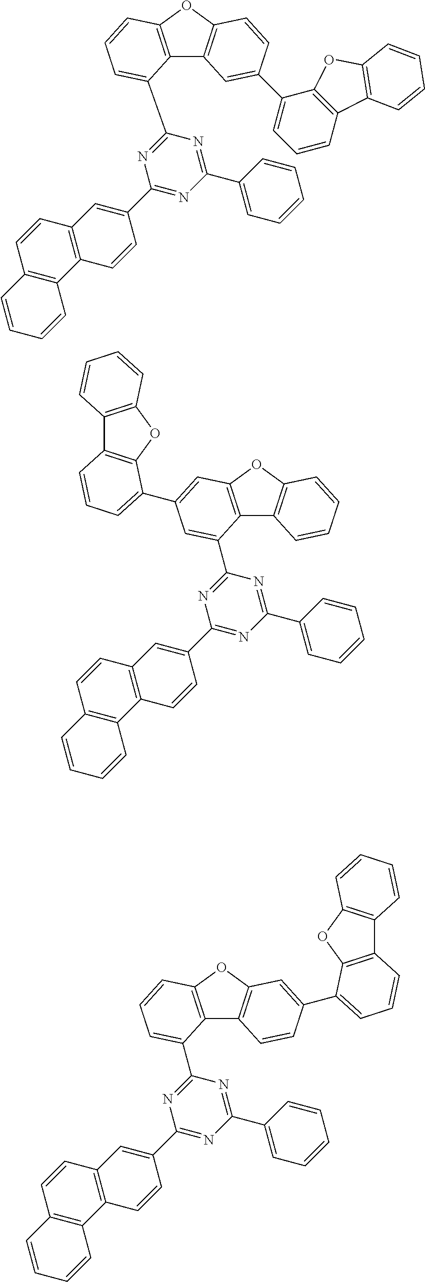 [SMILES: c1ccc(-c2nc(-c3ccc4c(ccc5ccccc54)c3)nc(-c3cccc4oc5ccc(-c6cccc7c6oc6ccccc67)cc5c34)n2)cc1 ; c1ccc(-c2nc(-c3ccc4c(ccc5ccccc54)c3)nc(-c3cc(-c4cccc5c4oc4ccccc45)cc4oc5ccccc5c34)n2)cc1 ; c1ccc(-c2nc(-c3ccc4c(ccc5ccccc54)c3)nc(-c3cccc4oc5cc(-c6cccc7c6oc6ccccc67)ccc5c34)n2)cc1]

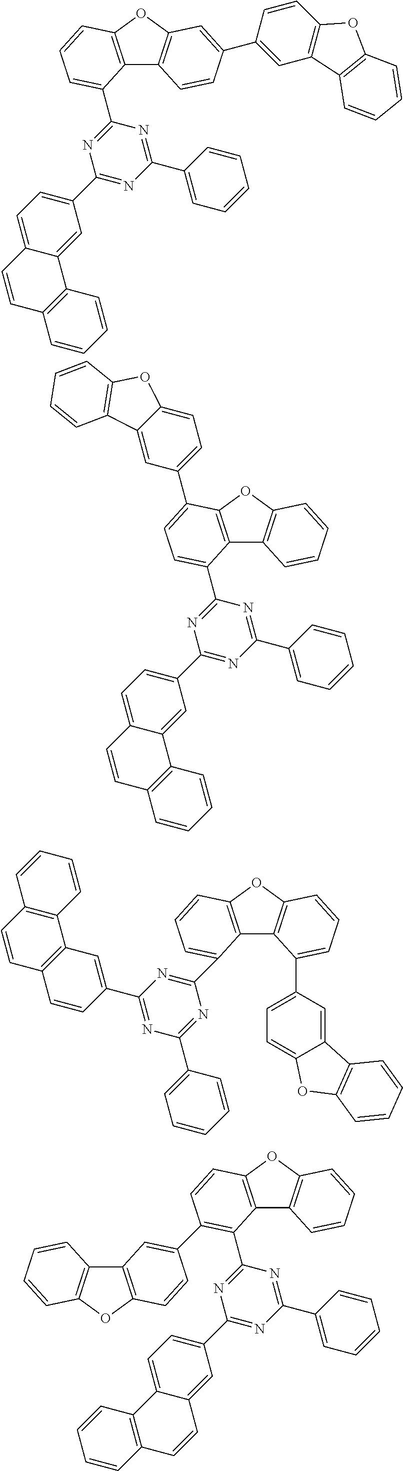

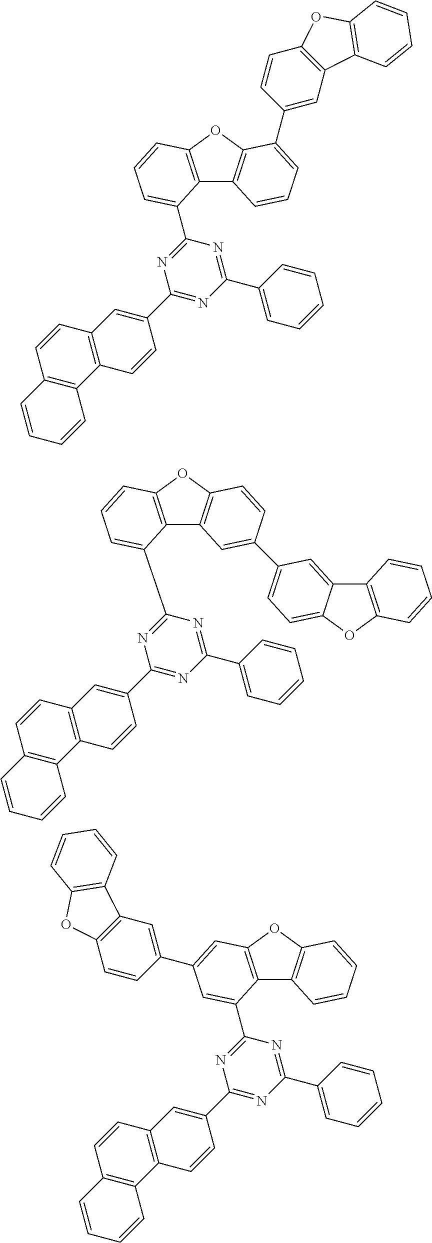

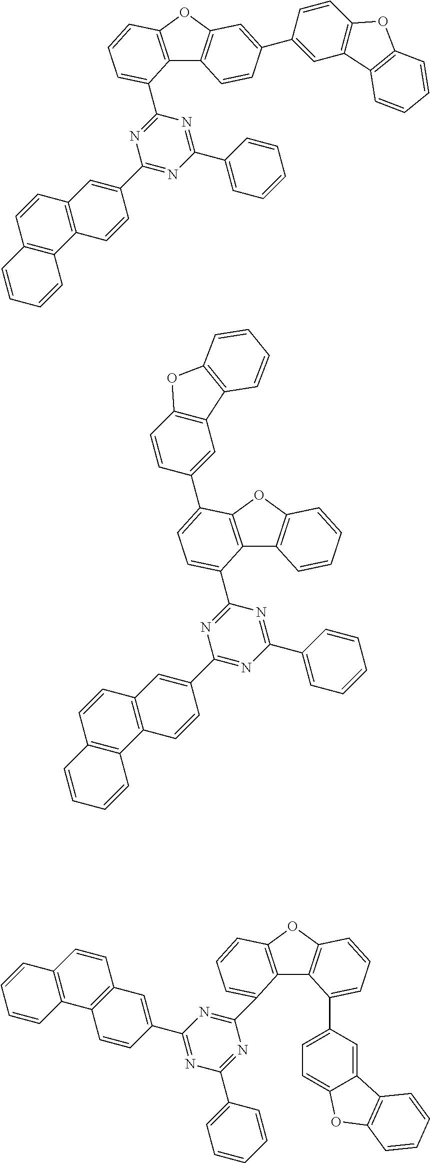 [SMILES: c1ccc(-c2nc(-c3ccc4c(ccc5ccccc54)c3)nc(-c3cccc4oc5cc(-c6ccc7oc8ccccc8c7c6)ccc5c34)n2)cc1 ; c1ccc(-c2nc(-c3ccc4c(ccc5ccccc54)c3)nc(-c3ccc(-c4ccc5oc6ccccc6c5c4)c4oc5ccccc5c34)n2)cc1 ; c1ccc(-c2nc(-c3ccc4c(ccc5ccccc54)c3)nc(-c3cccc4oc5cccc(-c6ccc7oc8ccccc8c7c6)c5c34)n2)cc1]

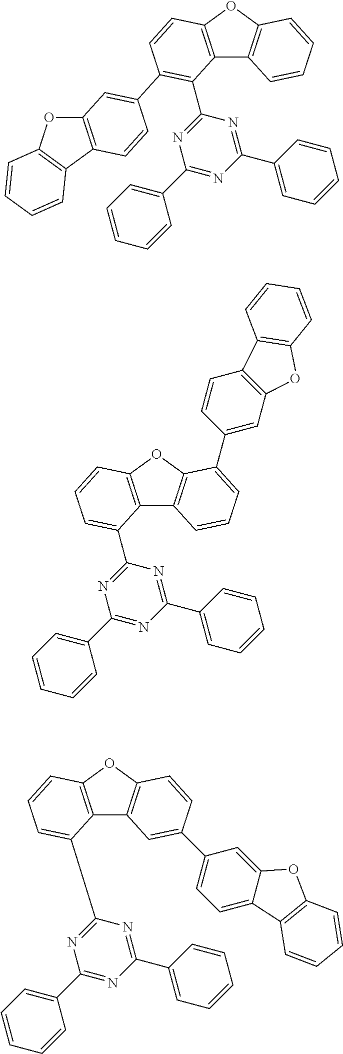 [SMILES: c1ccc(-c2nc(-c3ccccc3)nc(-c3c(-c4ccc5c(c4)oc4ccccc45)ccc4oc5ccccc5c34)n2)cc1 ; c1ccc(-c2nc(-c3ccccc3)nc(-c3cccc4oc5c(-c6ccc7c(c6)oc6ccccc67)cccc5c34)n2)cc1 ; c1ccc(-c2nc(-c3ccccc3)nc(-c3cccc4oc5ccc(-c6ccc7c(c6)oc6ccccc67)cc5c34)n2)cc1]

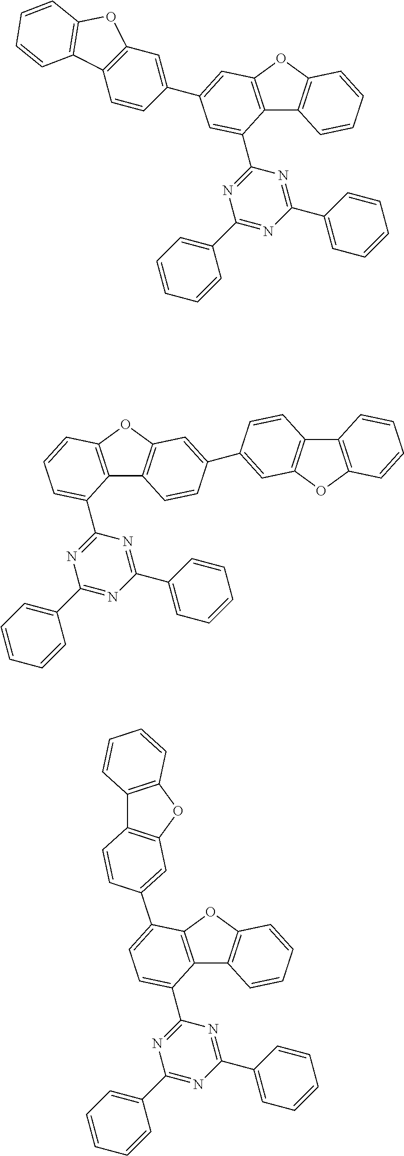 [SMILES: c1ccc(-c2nc(-c3ccccc3)nc(-c3cc(-c4ccc5c(c4)oc4ccccc45)cc4oc5ccccc5c34)n2)cc1 ; c1ccc(-c2nc(-c3ccccc3)nc(-c3cccc4oc5cc(-c6ccc7c(c6)oc6ccccc67)ccc5c34)n2)cc1 ; c1ccc(-c2nc(-c3ccccc3)nc(-c3ccc(-c4ccc5c(c4)oc4ccccc45)c4oc5ccccc5c34)n2)cc1]

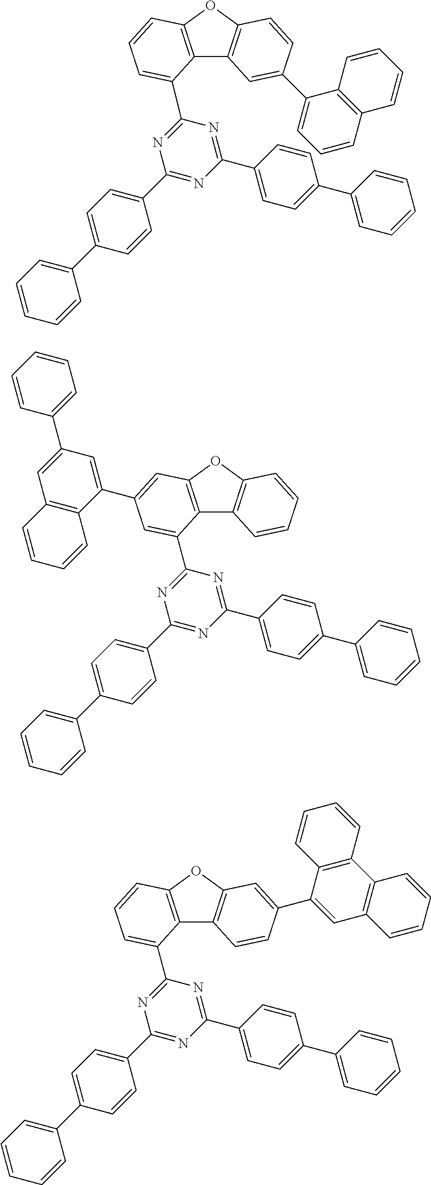

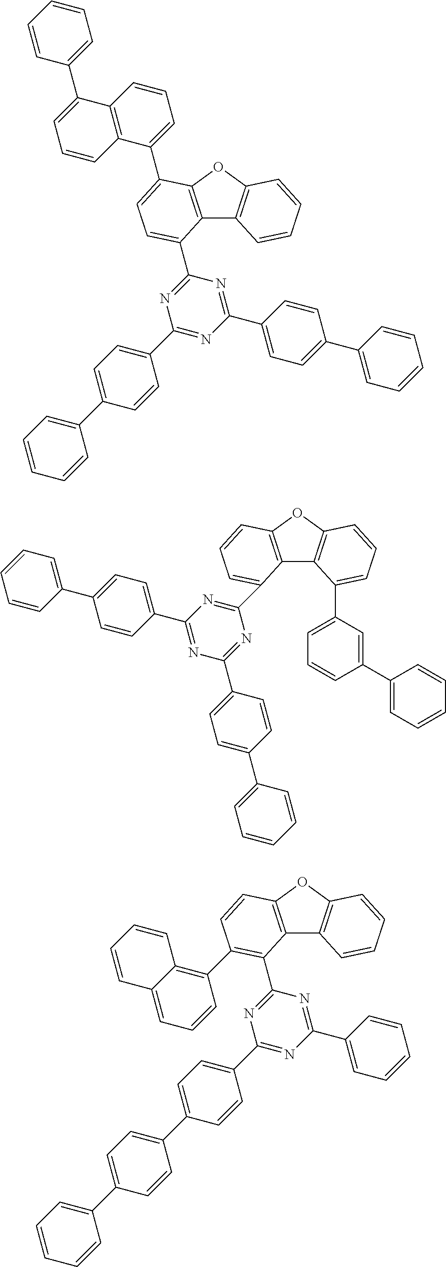 [SMILES: c1ccc(-c2ccc(-c3nc(-c4ccc(-c5ccccc5)cc4)nc(-c4ccc(-c5cccc6c(-c7ccccc7)cccc56)c5oc6ccccc6c45)n3)cc2)cc1 ; c1ccc(-c2ccc(-c3nc(-c4ccc(-c5ccccc5)cc4)nc(-c4cccc5oc6cccc(-c7cccc(-c8ccccc8)c7)c6c45)n3)cc2)cc1 ; c1ccc(-c2ccc(-c3ccc(-c4nc(-c5ccccc5)nc(-c5c(-c6cccc7ccccc67)ccc6oc7ccccc7c56)n4)cc3)cc2)cc1]

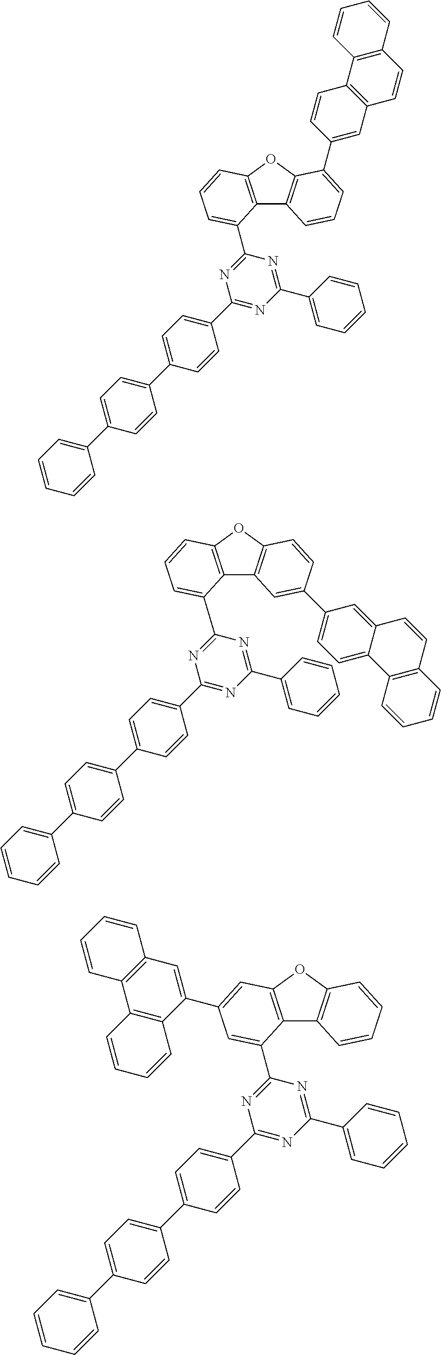 [SMILES: c1ccc(-c2ccc(-c3ccc(-c4nc(-c5ccccc5)nc(-c5cccc6oc7c(-c8ccc9c(ccc%10ccccc%109)c8)cccc7c56)n4)cc3)cc2)cc1 ; c1ccc(-c2ccc(-c3ccc(-c4nc(-c5ccccc5)nc(-c5cccc6oc7ccc(-c8ccc9c(ccc%10ccccc%109)c8)cc7c56)n4)cc3)cc2)cc1 ; c1ccc(-c2ccc(-c3ccc(-c4nc(-c5ccccc5)nc(-c5cc(-c6cc7ccccc7c7ccccc67)cc6oc7ccccc7c56)n4)cc3)cc2)cc1]

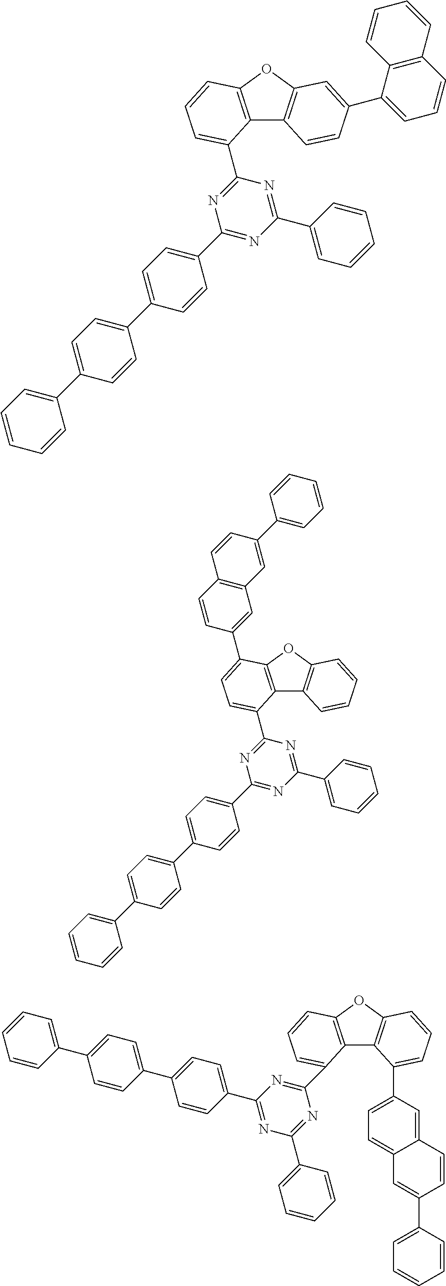 [SMILES: c1ccc(-c2ccc(-c3ccc(-c4nc(-c5ccccc5)nc(-c5cccc6oc7cc(-c8cccc9ccccc89)ccc7c56)n4)cc3)cc2)cc1 ; c1ccc(-c2ccc(-c3ccc(-c4nc(-c5ccccc5)nc(-c5ccc(-c6ccc7ccc(-c8ccccc8)cc7c6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9cc(-c%10ccccc%10)ccc9c8)c7c56)n4)cc3)cc2)cc1]

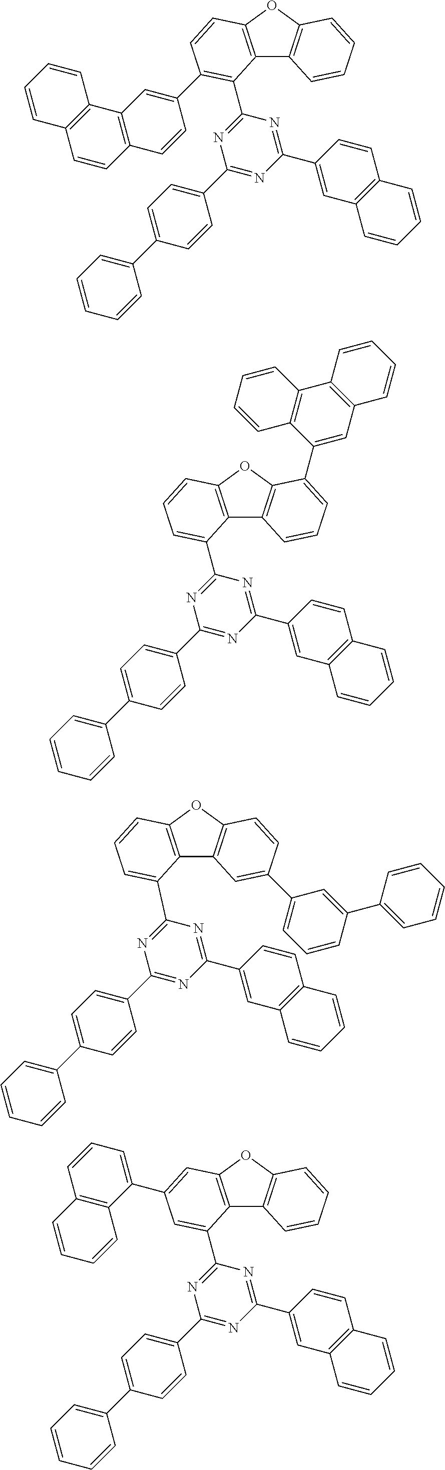 [SMILES: c1ccc(-c2ccc(-c3nc(-c4ccc5ccccc5c4)nc(-c4c(-c5ccc6ccc7ccccc7c6c5)ccc5oc6ccccc6c45)n3)cc2)cc1 ; c1ccc(-c2ccc(-c3nc(-c4ccc5ccccc5c4)nc(-c4cccc5oc6c(-c7cc8ccccc8c8ccccc78)cccc6c45)n3)cc2)cc1 ; c1ccc(-c2ccc(-c3nc(-c4ccc5ccccc5c4)nc(-c4cccc5oc6ccc(-c7cccc(-c8ccccc8)c7)cc6c45)n3)cc2)cc1 ; c1ccc(-c2ccc(-c3nc(-c4ccc5ccccc5c4)nc(-c4cc(-c5cccc6ccccc56)cc5oc6ccccc6c45)n3)cc2)cc1]

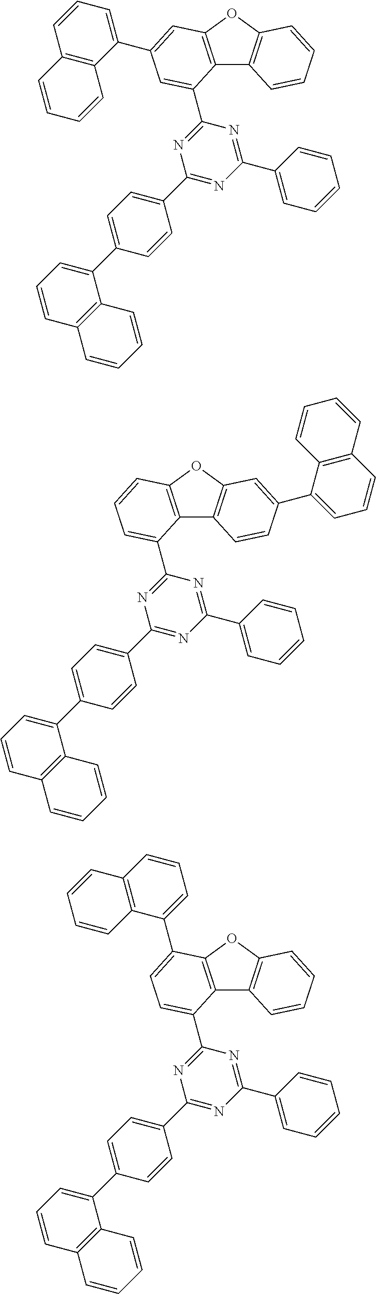 [SMILES: c1ccc(-c2nc(-c3ccc(-c4cccc5ccccc45)cc3)nc(-c3cc(-c4cccc5ccccc45)cc4oc5ccccc5c34)n2)cc1 ; c1ccc(-c2nc(-c3ccc(-c4cccc5ccccc45)cc3)nc(-c3cccc4oc5cc(-c6cccc7ccccc67)ccc5c34)n2)cc1 ; c1ccc(-c2nc(-c3ccc(-c4cccc5ccccc45)cc3)nc(-c3ccc(-c4cccc5ccccc45)c4oc5ccccc5c34)n2)cc1]

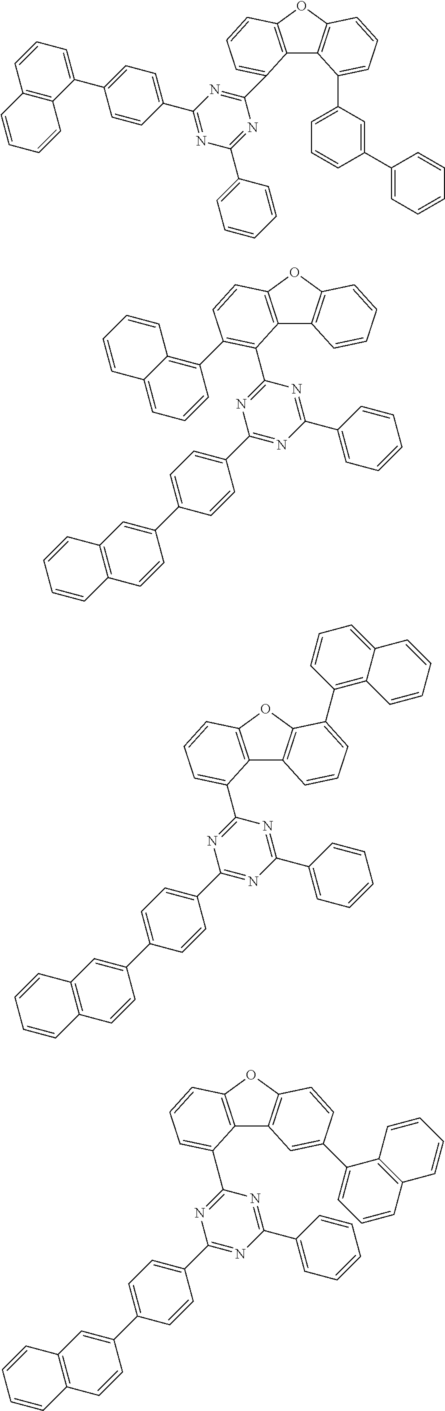 [SMILES: c1ccc(-c2cccc(-c3cccc4oc5cccc(-c6nc(-c7ccccc7)nc(-c7ccc(-c8cccc9ccccc89)cc7)n6)c5c34)c2)cc1 ; c1ccc(-c2nc(-c3ccc(-c4ccc5ccccc5c4)cc3)nc(-c3c(-c4cccc5ccccc45)ccc4oc5ccccc5c34)n2)cc1 ; c1ccc(-c2nc(-c3ccc(-c4ccc5ccccc5c4)cc3)nc(-c3cccc4oc5c(-c6cccc7ccccc67)cccc5c34)n2)cc1 ; c1ccc(-c2nc(-c3ccc(-c4ccc5ccccc5c4)cc3)nc(-c3cccc4oc5ccc(-c6cccc7ccccc67)cc5c34)n2)cc1]

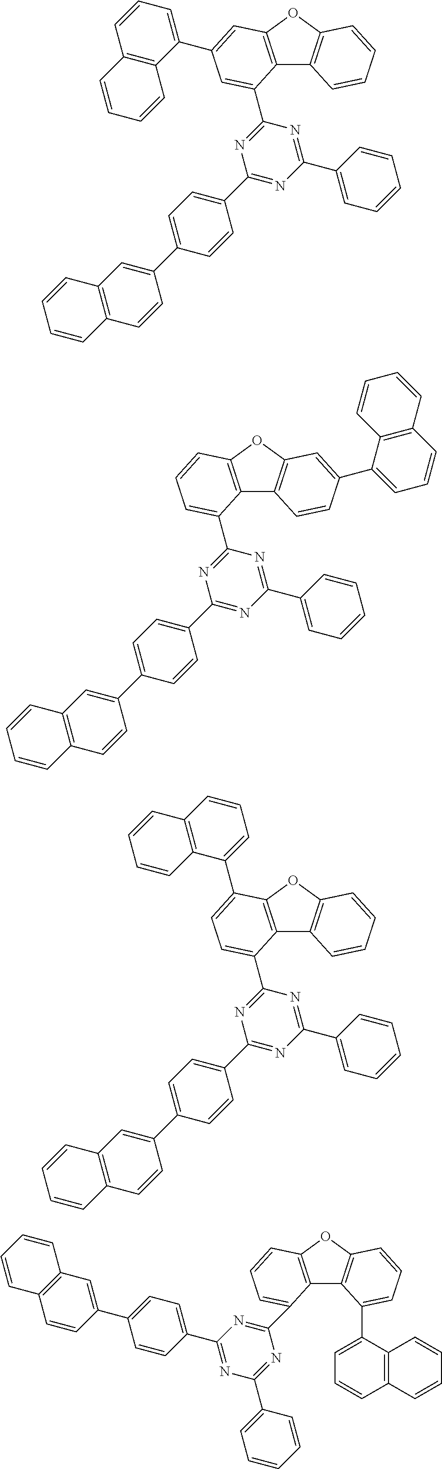

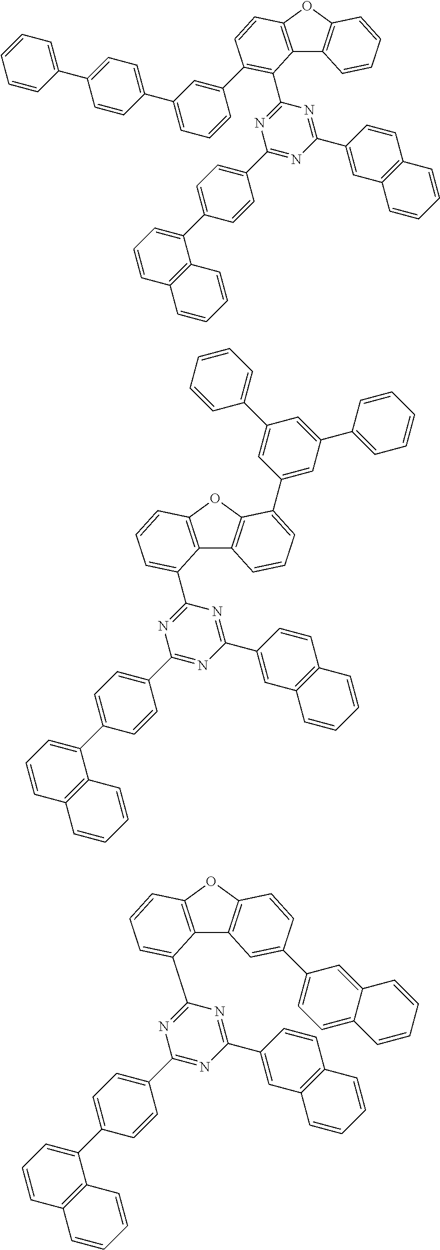

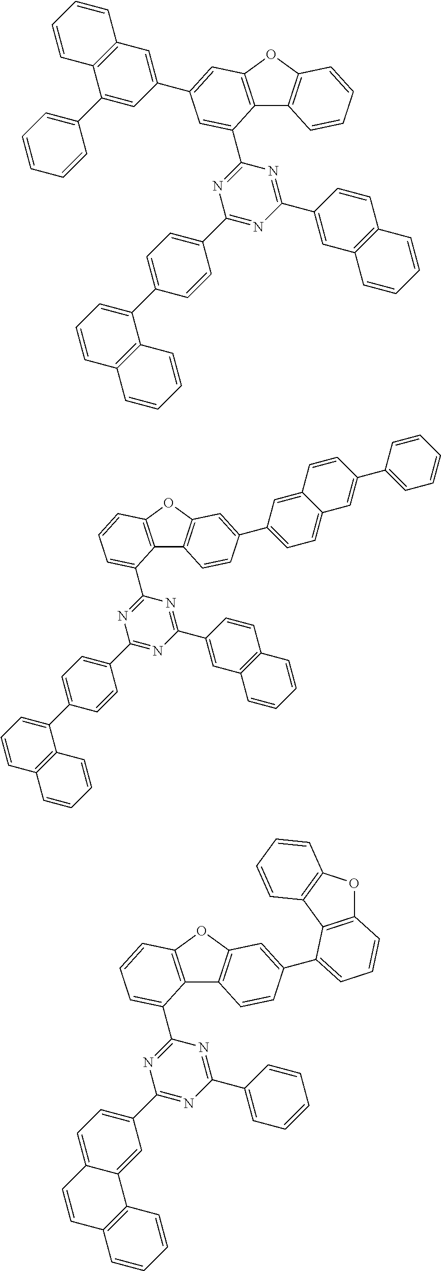 [SMILES: c1ccc(-c2cc(-c3cc(-c4nc(-c5ccc(-c6cccc7ccccc67)cc5)nc(-c5ccc6ccccc6c5)n4)c4c(c3)oc3ccccc34)cc3ccccc23)cc1 ; c1ccc(-c2ccc3cc(-c4ccc5c(c4)oc4cccc(-c6nc(-c7ccc(-c8cccc9ccccc89)cc7)nc(-c7ccc8ccccc8c7)n6)c45)ccc3c2)cc1 ; c1ccc(-c2nc(-c3ccc4ccc5ccccc5c4c3)nc(-c3cccc4oc5cc(-c6cccc7oc8ccccc8c67)ccc5c34)n2)cc1]

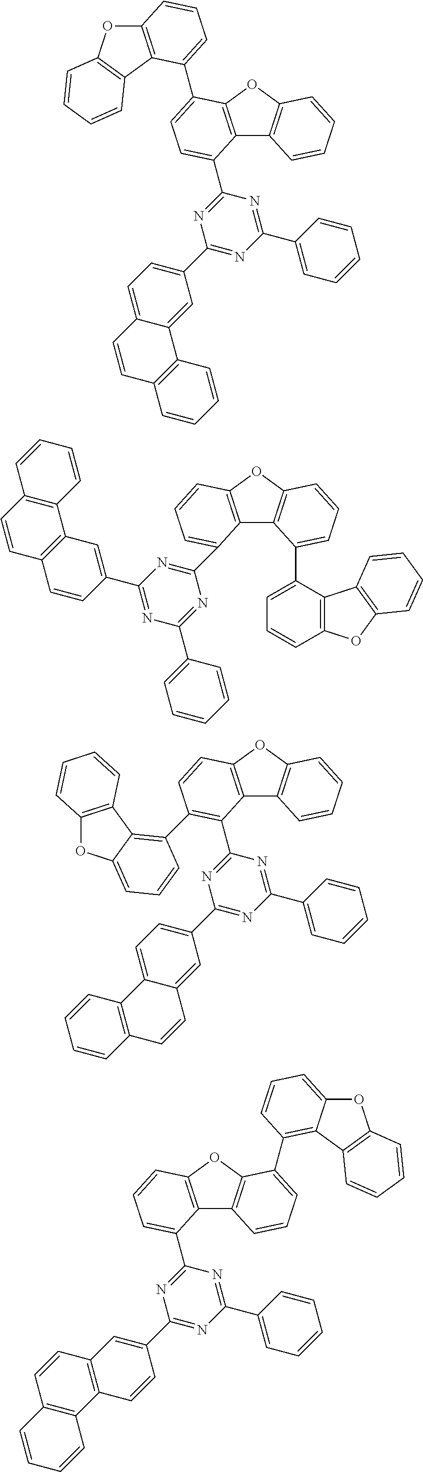

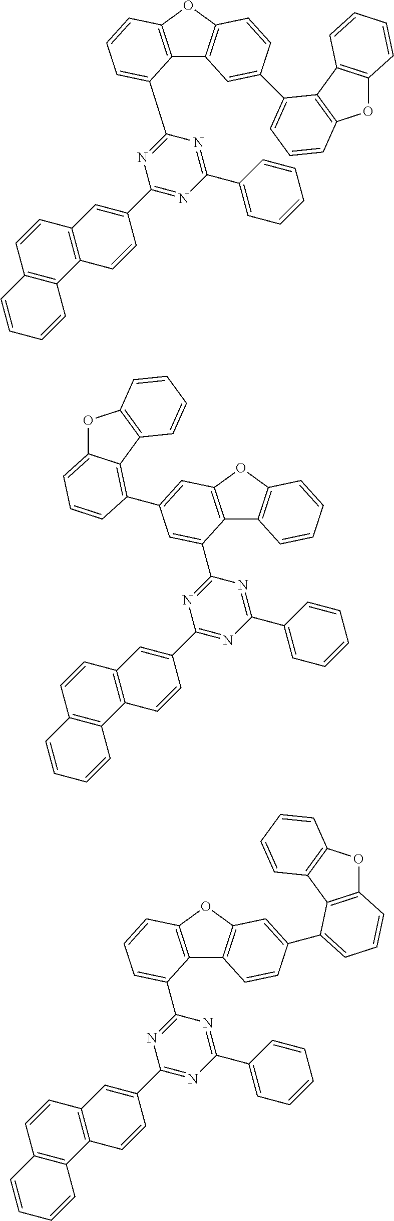 [SMILES: c1ccc(-c2nc(-c3ccc4c(ccc5ccccc54)c3)nc(-c3cccc4oc5ccc(-c6cccc7oc8ccccc8c67)cc5c34)n2)cc1 ; c1ccc(-c2nc(-c3ccc4c(ccc5ccccc54)c3)nc(-c3cc(-c4cccc5oc6ccccc6c45)cc4oc5ccccc5c34)n2)cc1 ; c1ccc(-c2nc(-c3ccc4c(ccc5ccccc54)c3)nc(-c3cccc4oc5cc(-c6cccc7oc8ccccc8c67)ccc5c34)n2)cc1]

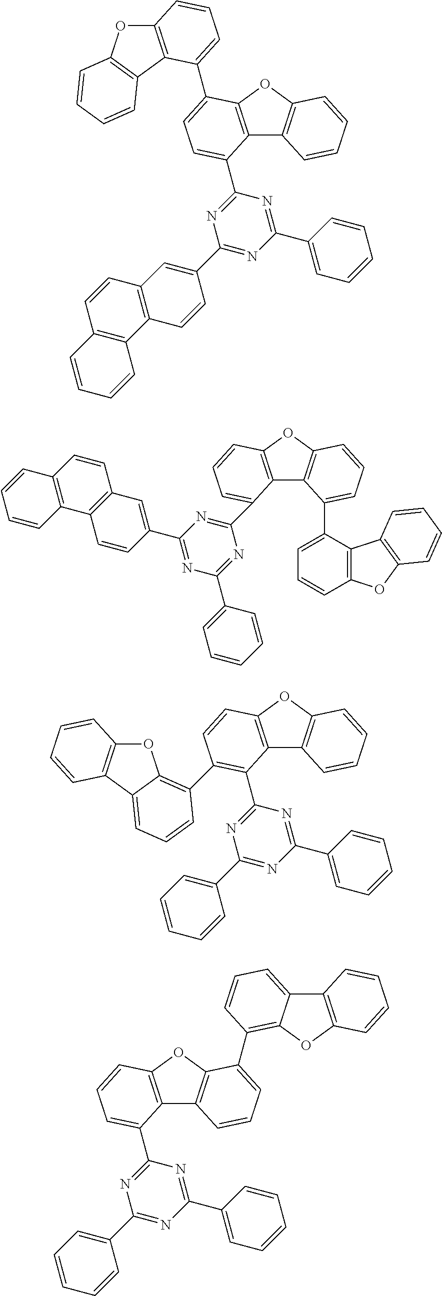

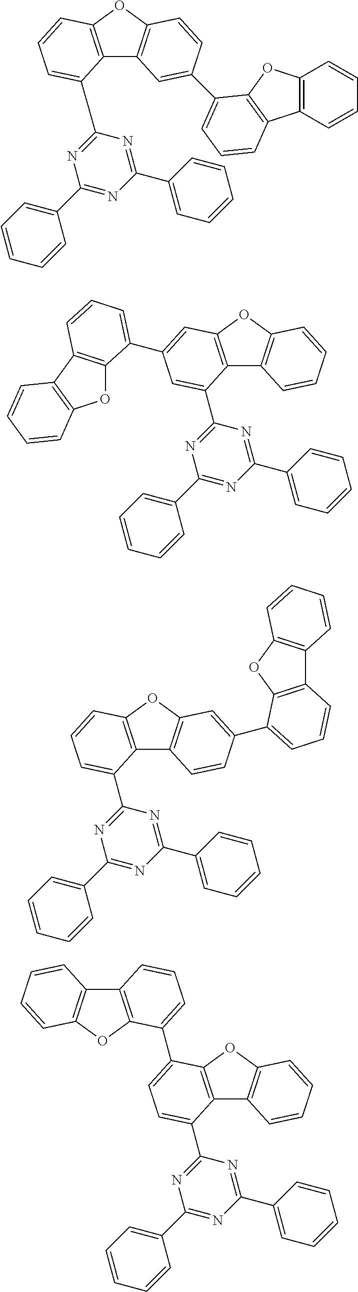

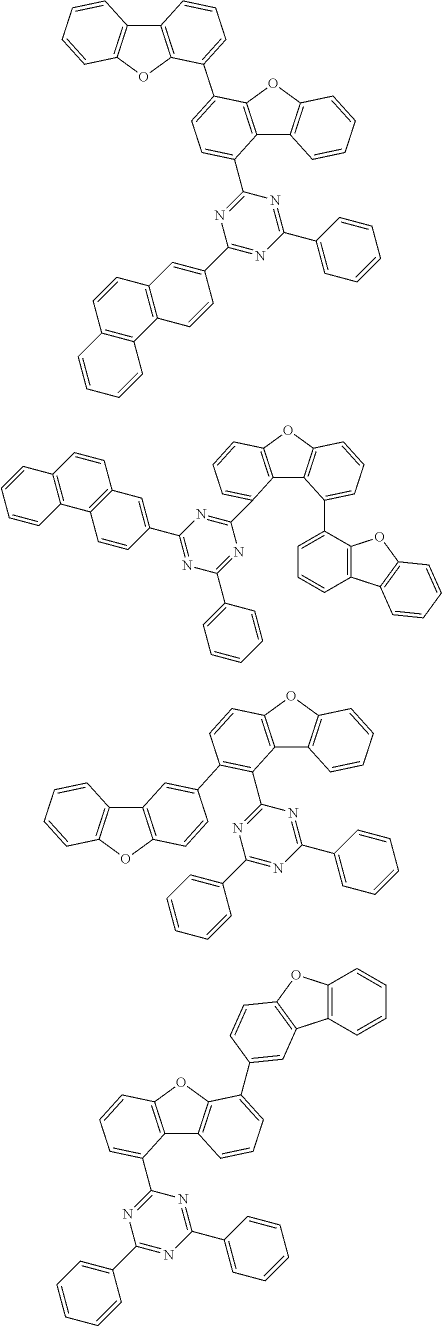 [SMILES: c1ccc(-c2nc(-c3ccc4c(ccc5ccccc54)c3)nc(-c3ccc(-c4cccc5c4oc4ccccc45)c4oc5ccccc5c34)n2)cc1 ; c1ccc(-c2nc(-c3ccc4c(ccc5ccccc54)c3)nc(-c3cccc4oc5cccc(-c6cccc7c6oc6ccccc67)c5c34)n2)cc1 ; c1ccc(-c2nc(-c3ccccc3)nc(-c3c(-c4ccc5oc6ccccc6c5c4)ccc4oc5ccccc5c34)n2)cc1 ; c1ccc(-c2nc(-c3ccccc3)nc(-c3cccc4oc5c(-c6ccc7oc8ccccc8c7c6)cccc5c34)n2)cc1]

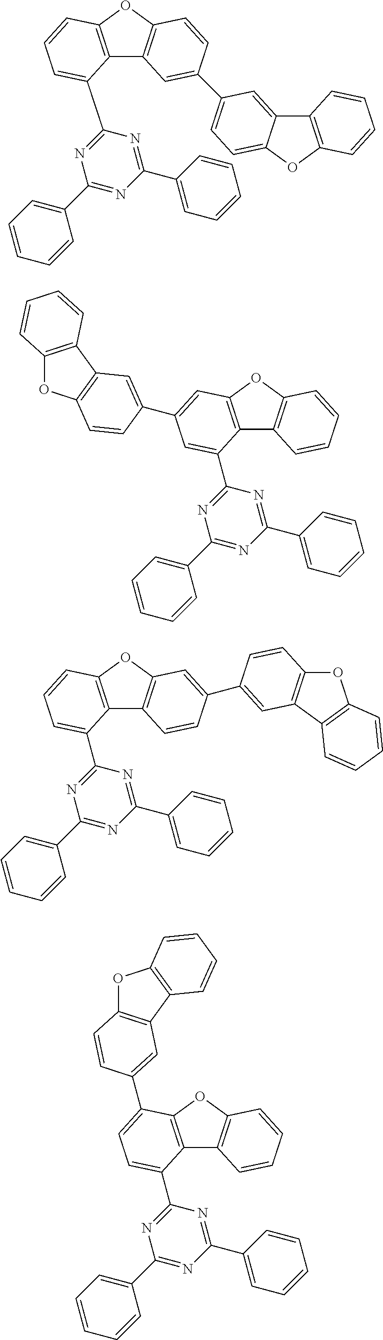

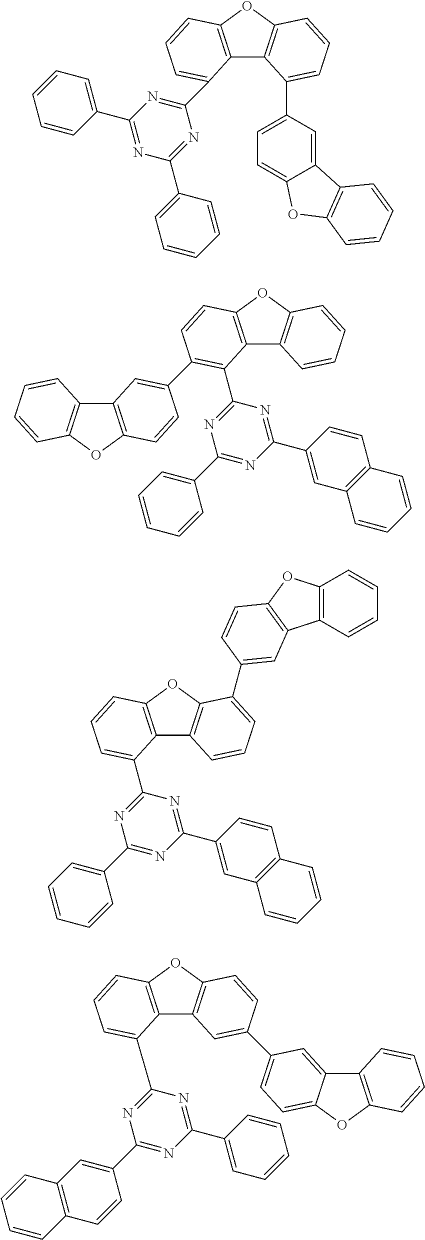 [SMILES: c1ccc(-c2nc(-c3ccccc3)nc(-c3cccc4oc5cccc(-c6ccc7oc8ccccc8c7c6)c5c34)n2)cc1 ; c1ccc(-c2nc(-c3ccc4ccccc4c3)nc(-c3c(-c4ccc5oc6ccccc6c5c4)ccc4oc5ccccc5c34)n2)cc1 ; c1ccc(-c2nc(-c3ccc4ccccc4c3)nc(-c3cccc4oc5c(-c6ccc7oc8ccccc8c7c6)cccc5c34)n2)cc1 ; c1ccc(-c2nc(-c3ccc4ccccc4c3)nc(-c3cccc4oc5ccc(-c6ccc7oc8ccccc8c7c6)cc5c34)n2)cc1]

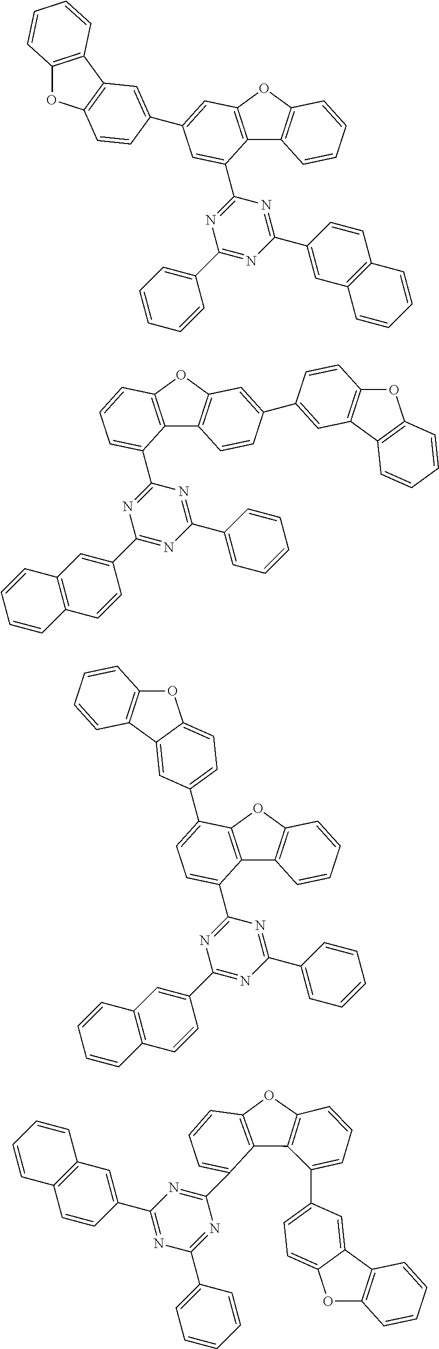

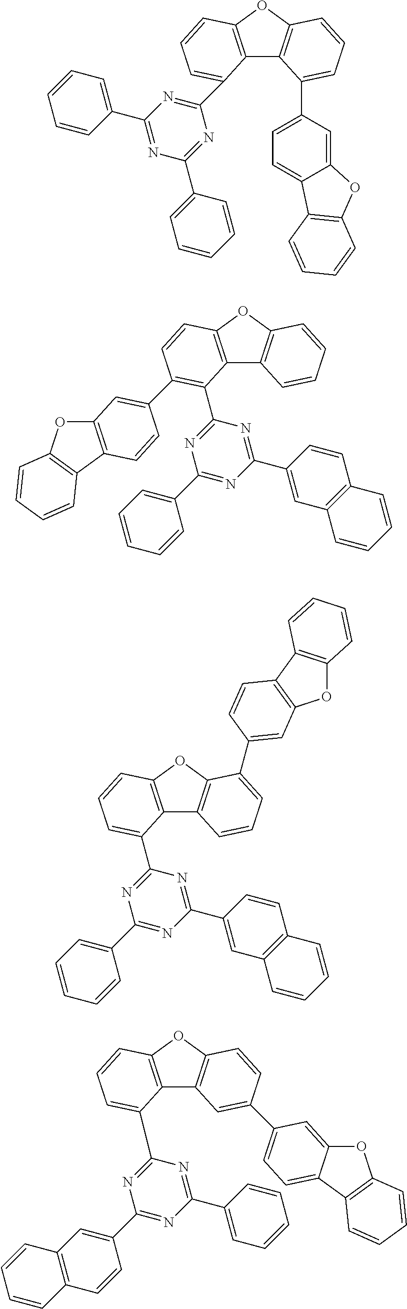 [SMILES: c1ccc(-c2nc(-c3ccccc3)nc(-c3cccc4oc5cccc(-c6ccc7c(c6)oc6ccccc67)c5c34)n2)cc1 ; c1ccc(-c2nc(-c3ccc4ccccc4c3)nc(-c3c(-c4ccc5c(c4)oc4ccccc45)ccc4oc5ccccc5c34)n2)cc1 ; c1ccc(-c2nc(-c3ccc4ccccc4c3)nc(-c3cccc4oc5c(-c6ccc7c(c6)oc6ccccc67)cccc5c34)n2)cc1 ; c1ccc(-c2nc(-c3ccc4ccccc4c3)nc(-c3cccc4oc5ccc(-c6ccc7c(c6)oc6ccccc67)cc5c34)n2)cc1]

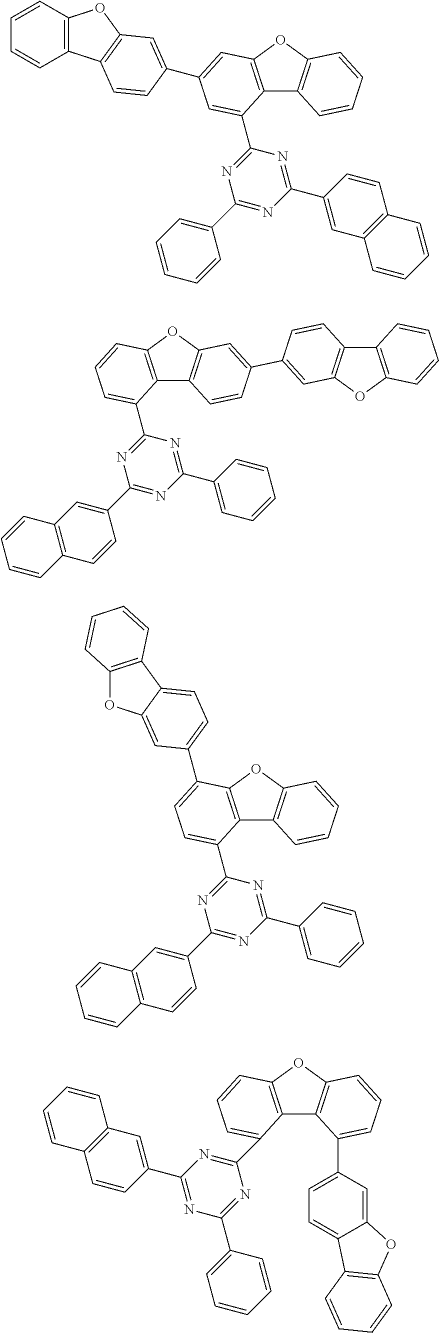

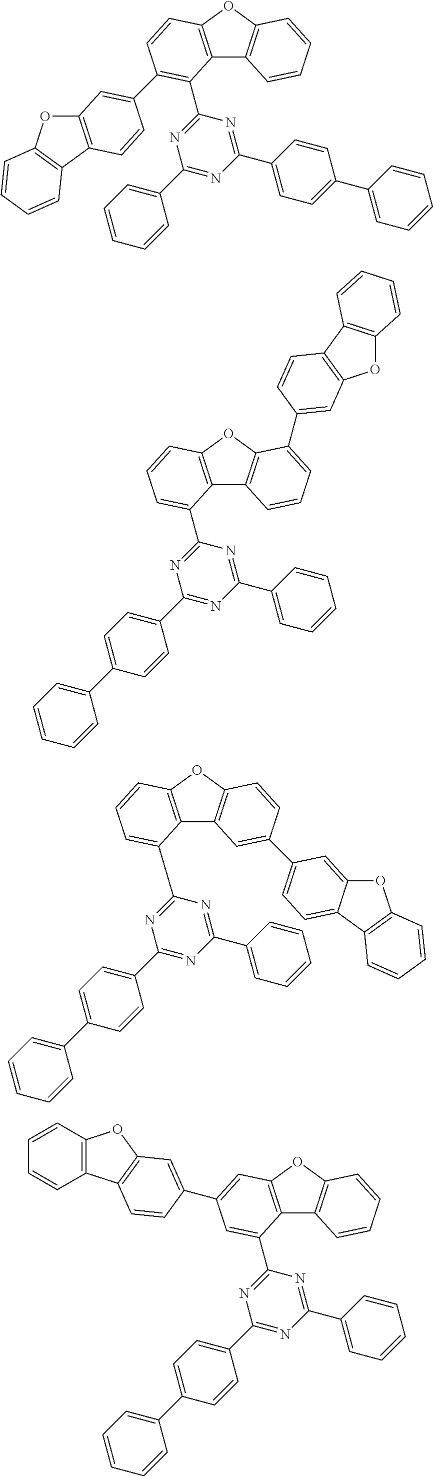

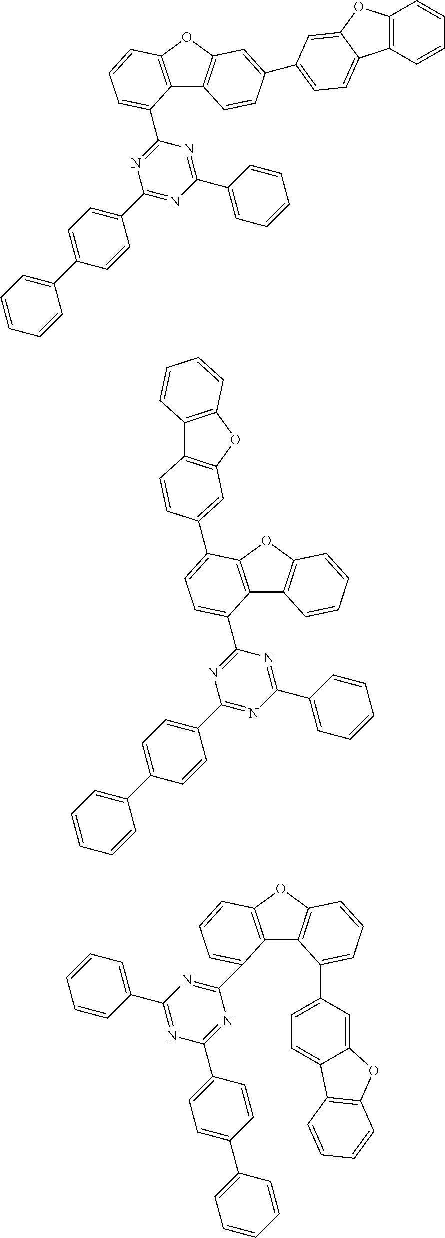 [SMILES: c1ccc(-c2ccc(-c3nc(-c4ccccc4)nc(-c4cccc5oc6cc(-c7ccc8c(c7)oc7ccccc78)ccc6c45)n3)cc2)cc1 ; c1ccc(-c2ccc(-c3nc(-c4ccccc4)nc(-c4ccc(-c5ccc6c(c5)oc5ccccc56)c5oc6ccccc6c45)n3)cc2)cc1 ; c1ccc(-c2ccc(-c3nc(-c4ccccc4)nc(-c4cccc5oc6cccc(-c7ccc8c(c7)oc7ccccc78)c6c45)n3)cc2)cc1]

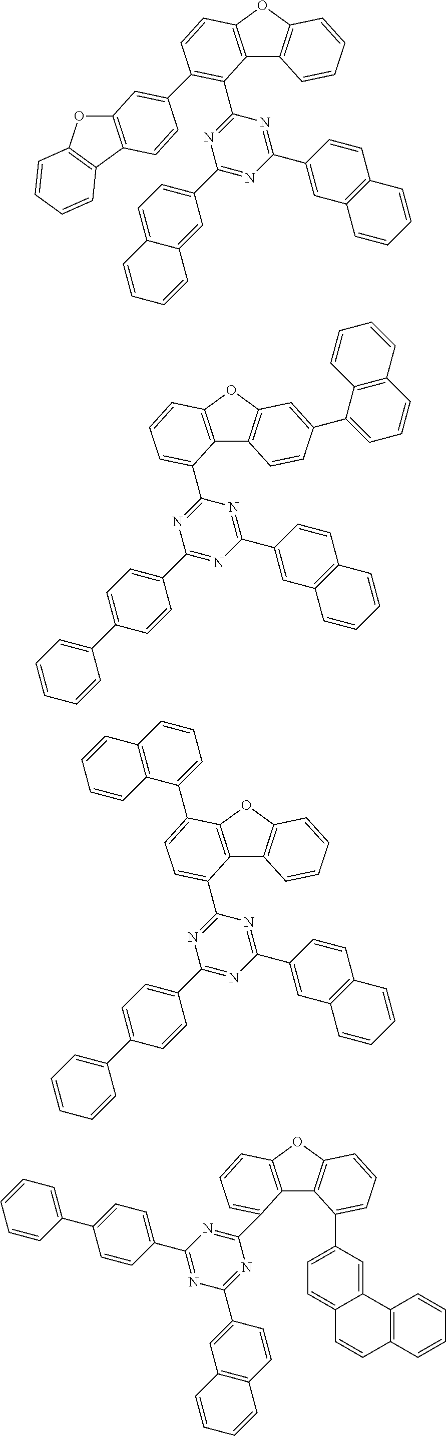

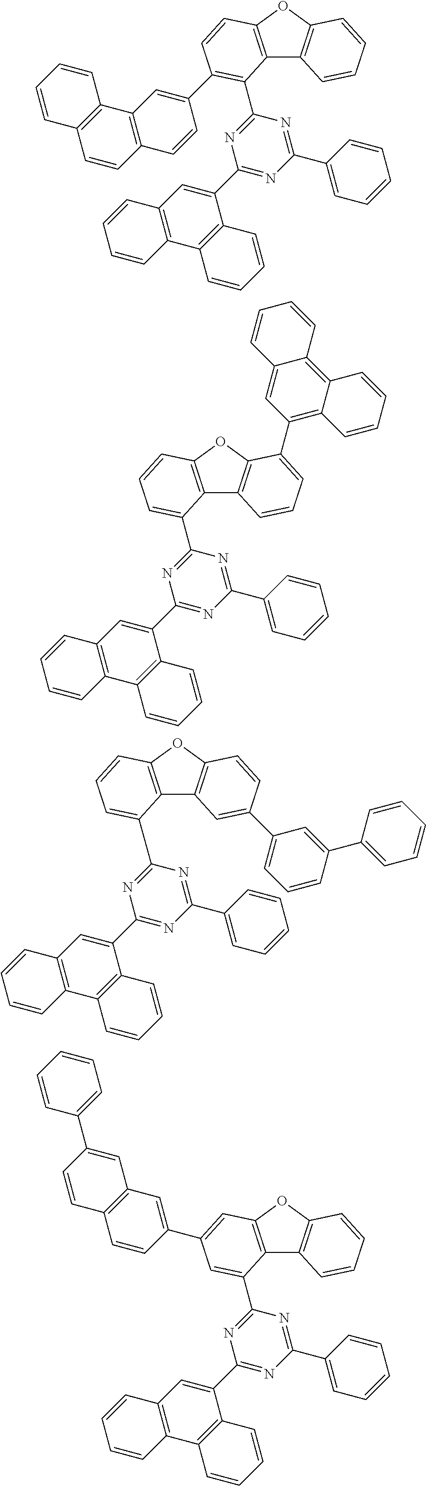

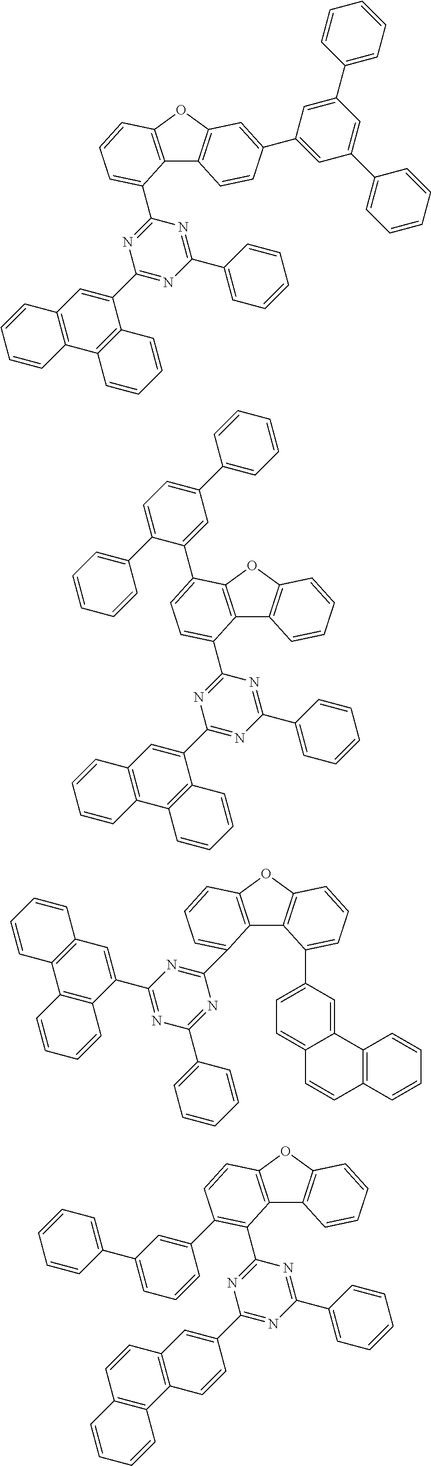

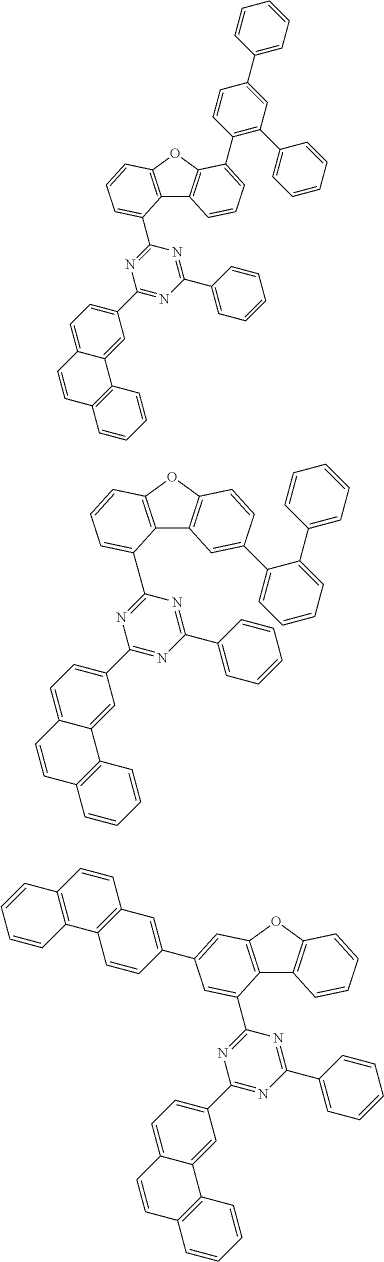 [SMILES: c1ccc(-c2ccc(-c3cccc4c3oc3cccc(-c5nc(-c6ccccc6)nc(-c6ccc7ccc8ccccc8c7c6)n5)c34)c(-c3ccccc3)c2)cc1 ; c1ccc(-c2nc(-c3ccc4ccc5ccccc5c4c3)nc(-c3cccc4oc5ccc(-c6ccccc6-c6ccccc6)cc5c34)n2)cc1 ; c1ccc(-c2nc(-c3ccc4ccc5ccccc5c4c3)nc(-c3cc(-c4ccc5c(ccc6ccccc65)c4)cc4oc5ccccc5c34)n2)cc1]

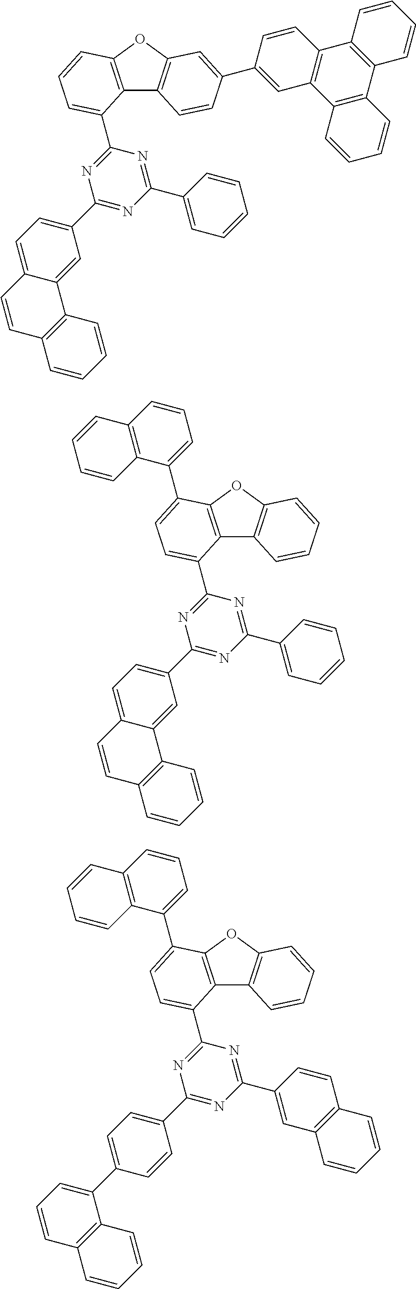

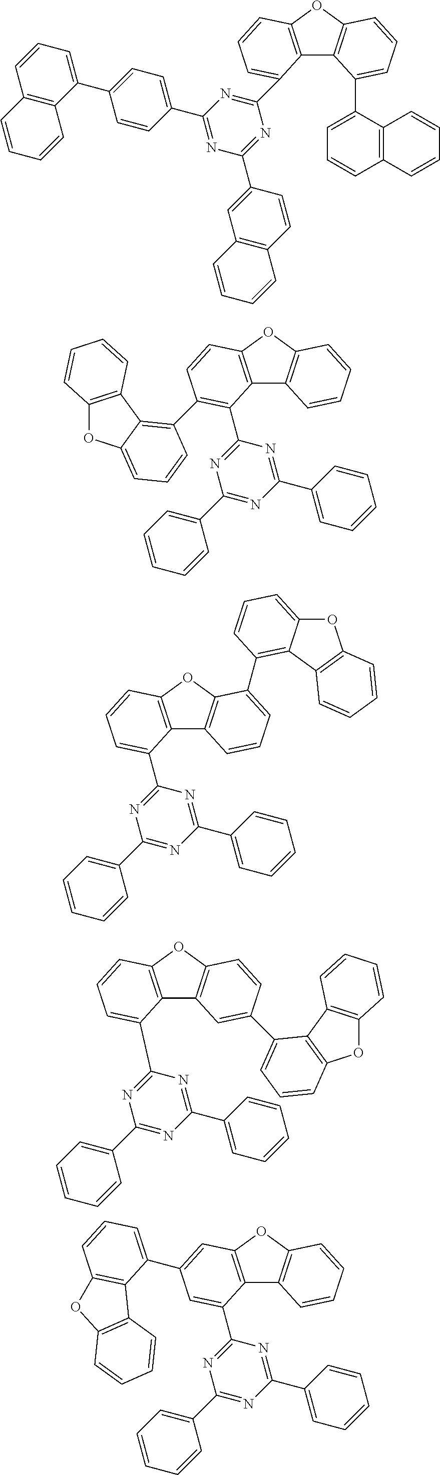

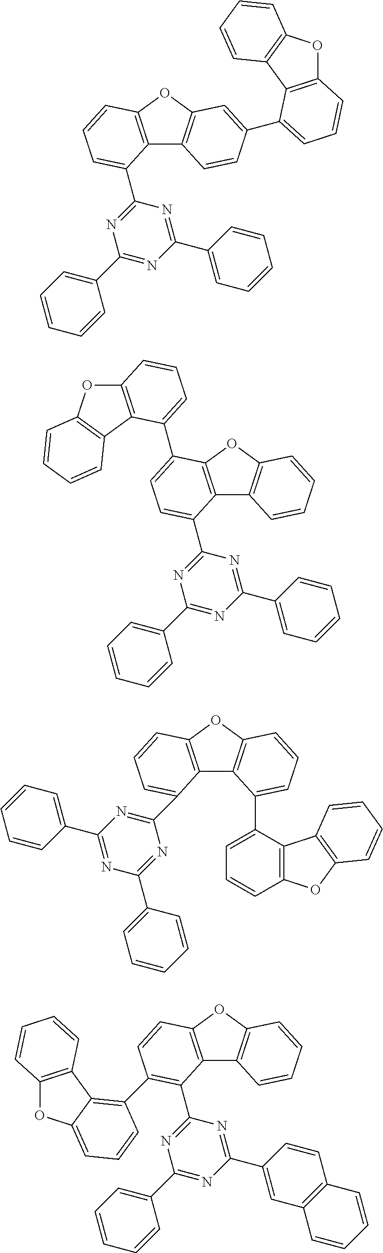

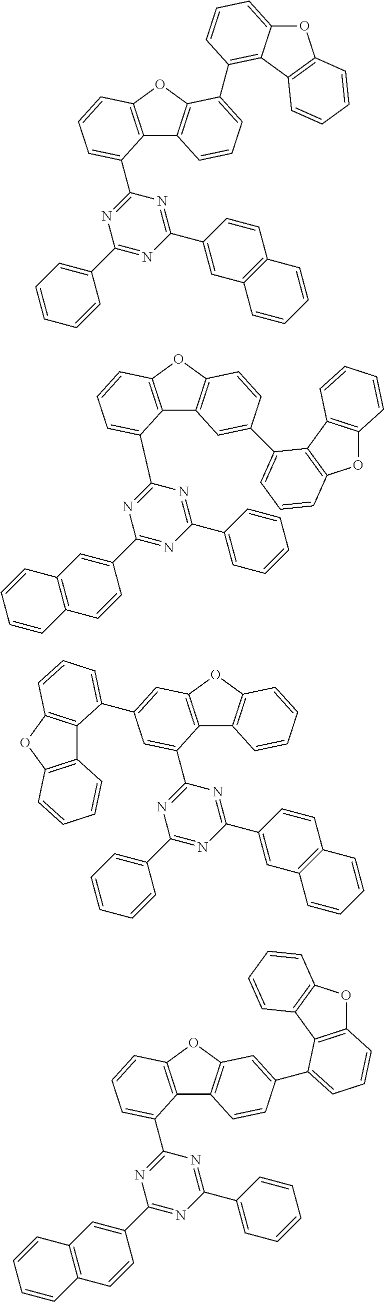

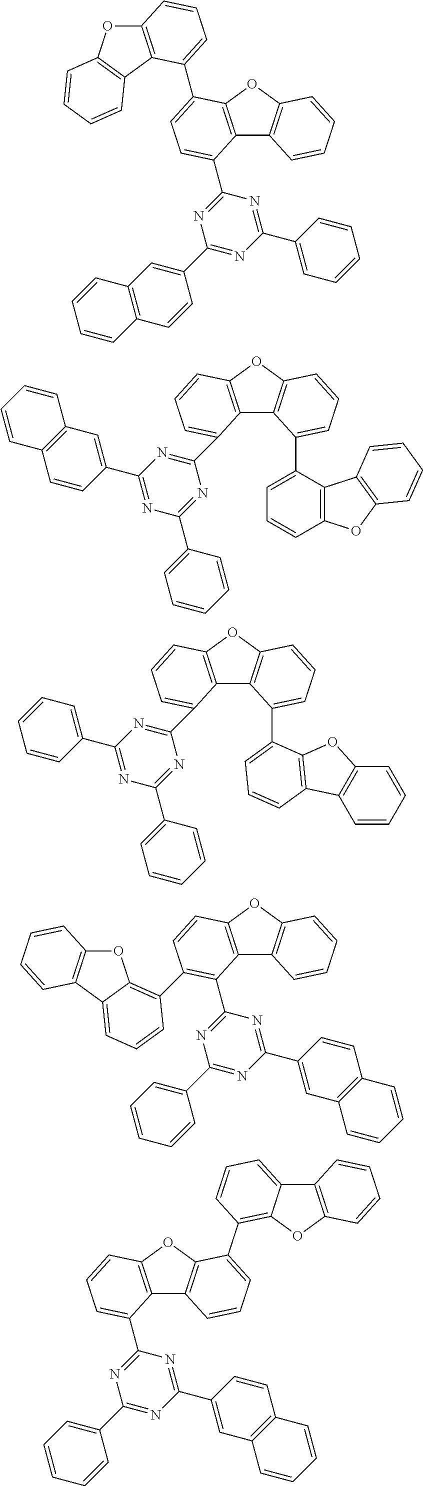

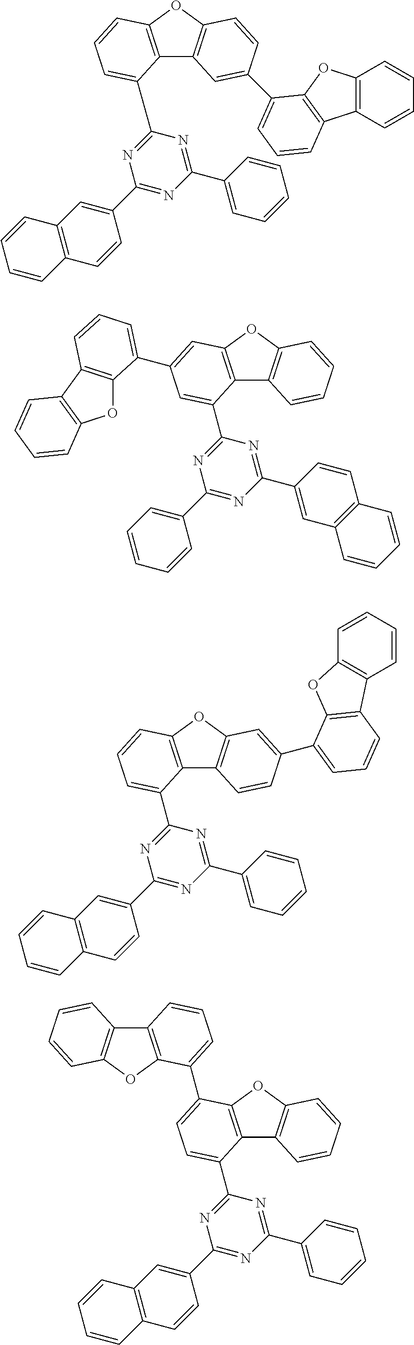

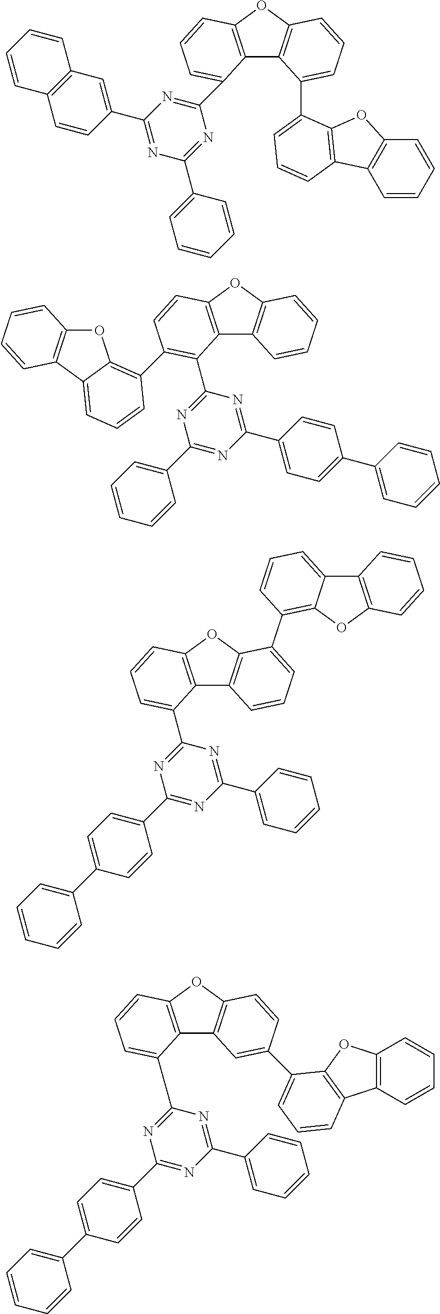

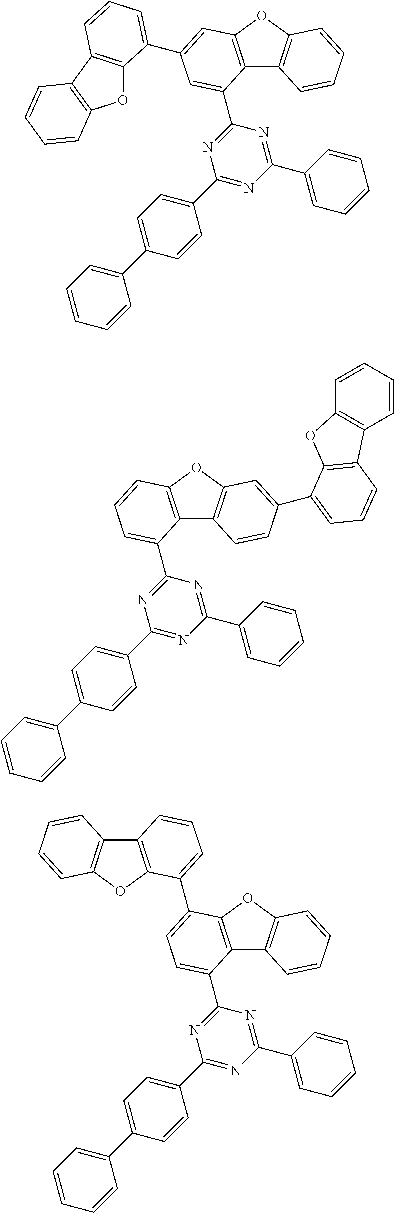

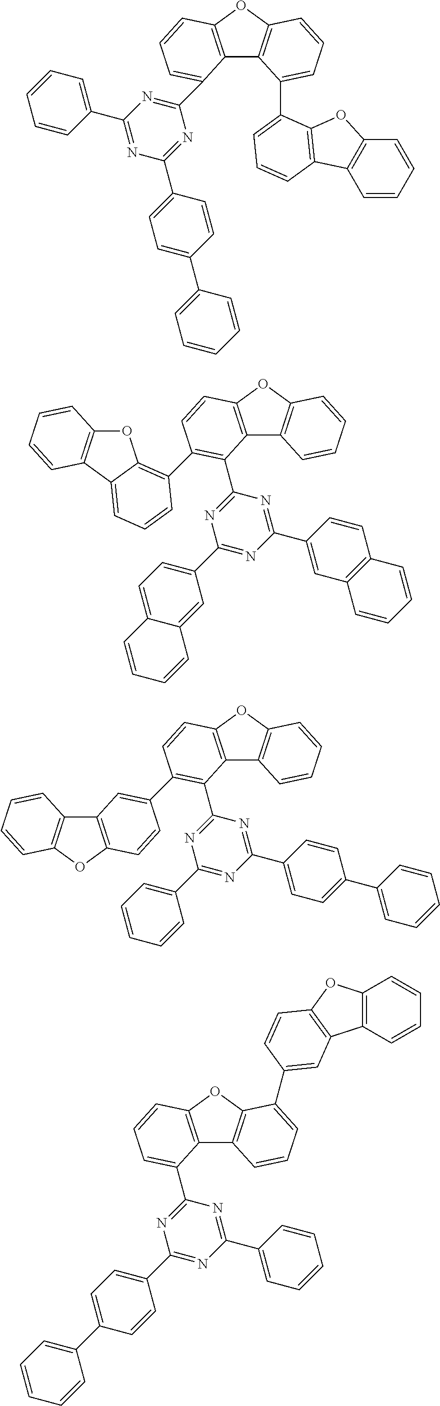

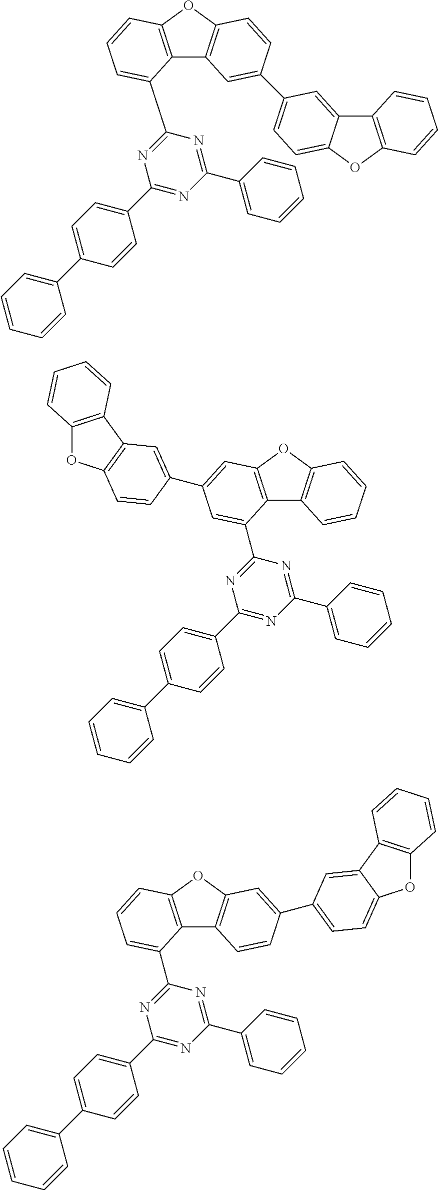

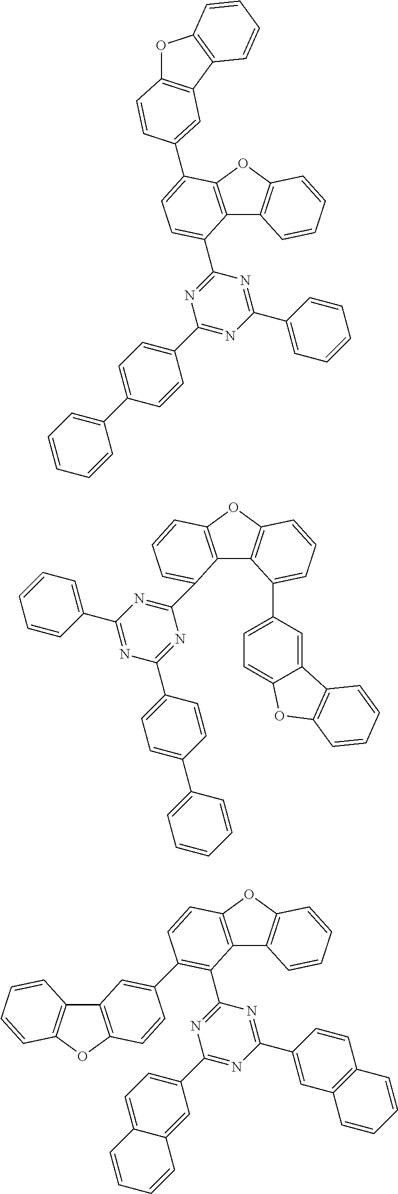 [SMILES: c1ccc(-c2ccc(-c3nc(-c4ccccc4)nc(-c4ccc(-c5ccc6oc7ccccc7c6c5)c5oc6ccccc6c45)n3)cc2)cc1 ; c1ccc(-c2ccc(-c3nc(-c4ccccc4)nc(-c4cccc5oc6cccc(-c7ccc8oc9ccccc9c8c7)c6c45)n3)cc2)cc1 ; c1ccc2cc(-c3nc(-c4ccc5ccccc5c4)nc(-c4c(-c5ccc6oc7ccccc7c6c5)ccc5oc6ccccc6c45)n3)ccc2c1]

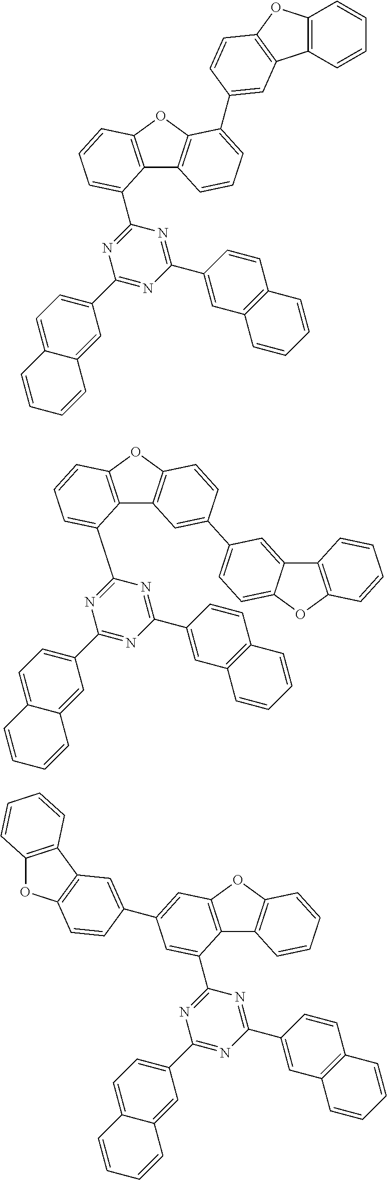

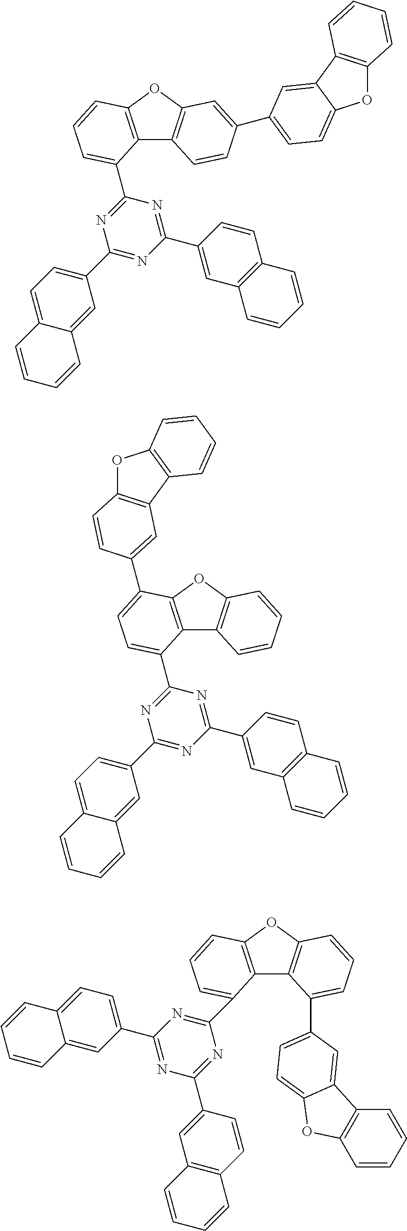 [SMILES: c1ccc2cc(-c3nc(-c4ccc5ccccc5c4)nc(-c4cccc5oc6cc(-c7ccc8oc9ccccc9c8c7)ccc6c45)n3)ccc2c1 ; c1ccc2cc(-c3nc(-c4ccc5ccccc5c4)nc(-c4ccc(-c5ccc6oc7ccccc7c6c5)c5oc6ccccc6c45)n3)ccc2c1 ; c1ccc2cc(-c3nc(-c4ccc5ccccc5c4)nc(-c4cccc5oc6cccc(-c7ccc8oc9ccccc9c8c7)c6c45)n3)ccc2c1]

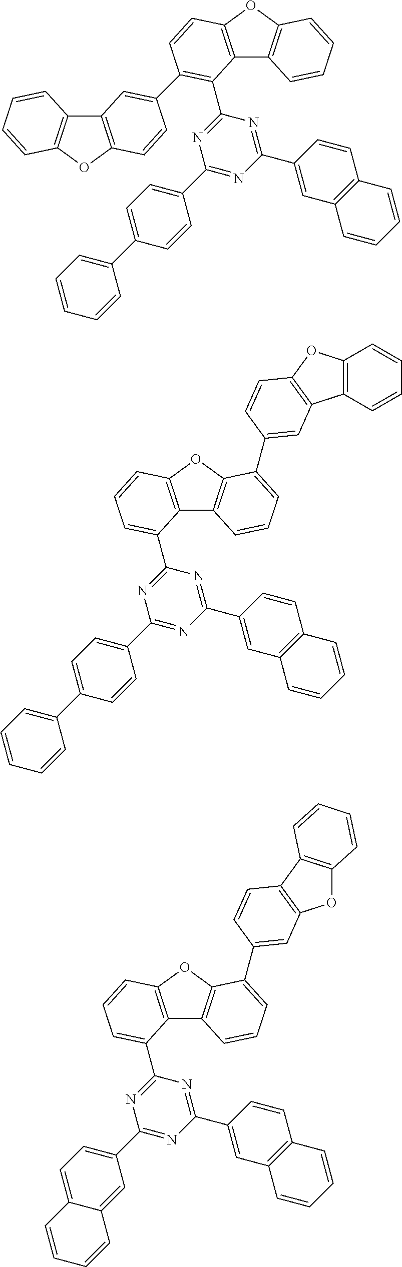 [SMILES: c1ccc(-c2ccc(-c3nc(-c4ccc5ccccc5c4)nc(-c4c(-c5ccc6oc7ccccc7c6c5)ccc5oc6ccccc6c45)n3)cc2)cc1 ; c1ccc(-c2ccc(-c3nc(-c4ccc5ccccc5c4)nc(-c4cccc5oc6c(-c7ccc8oc9ccccc9c8c7)cccc6c45)n3)cc2)cc1 ; c1ccc2cc(-c3nc(-c4ccc5ccccc5c4)nc(-c4cccc5oc6c(-c7ccc8c(c7)oc7ccccc78)cccc6c45)n3)ccc2c1]

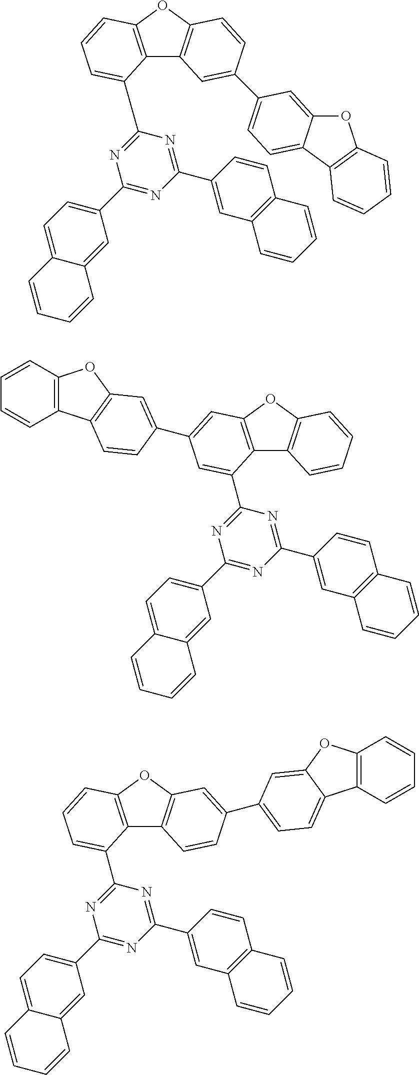 [SMILES: c1ccc2cc(-c3nc(-c4ccc5ccccc5c4)nc(-c4cccc5oc6ccc(-c7ccc8c(c7)oc7ccccc78)cc6c45)n3)ccc2c1 ; c1ccc2cc(-c3nc(-c4ccc5ccccc5c4)nc(-c4cc(-c5ccc6c(c5)oc5ccccc56)cc5oc6ccccc6c45)n3)ccc2c1 ; c1ccc2cc(-c3nc(-c4ccc5ccccc5c4)nc(-c4cccc5oc6cc(-c7ccc8c(c7)oc7ccccc78)ccc6c45)n3)ccc2c1]

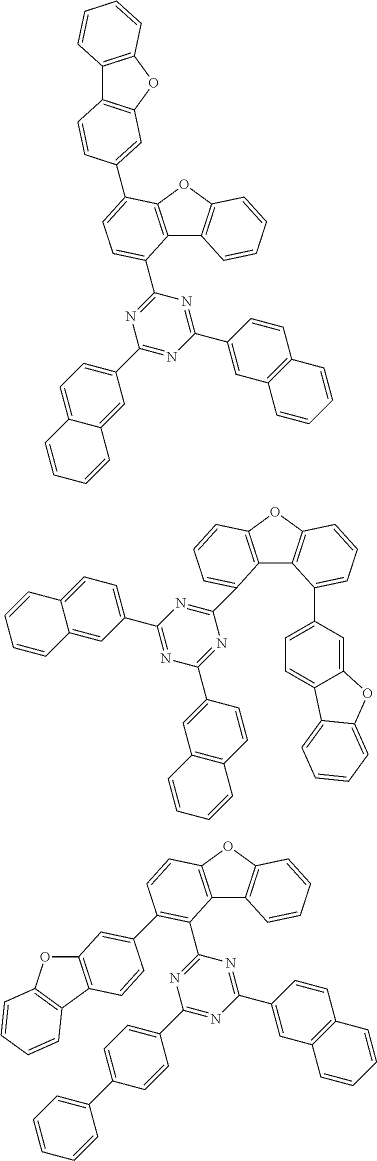

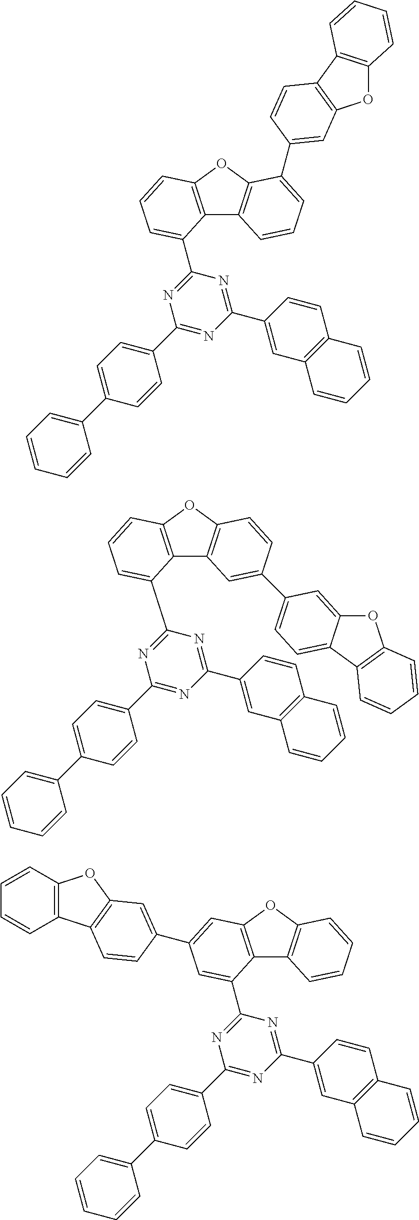

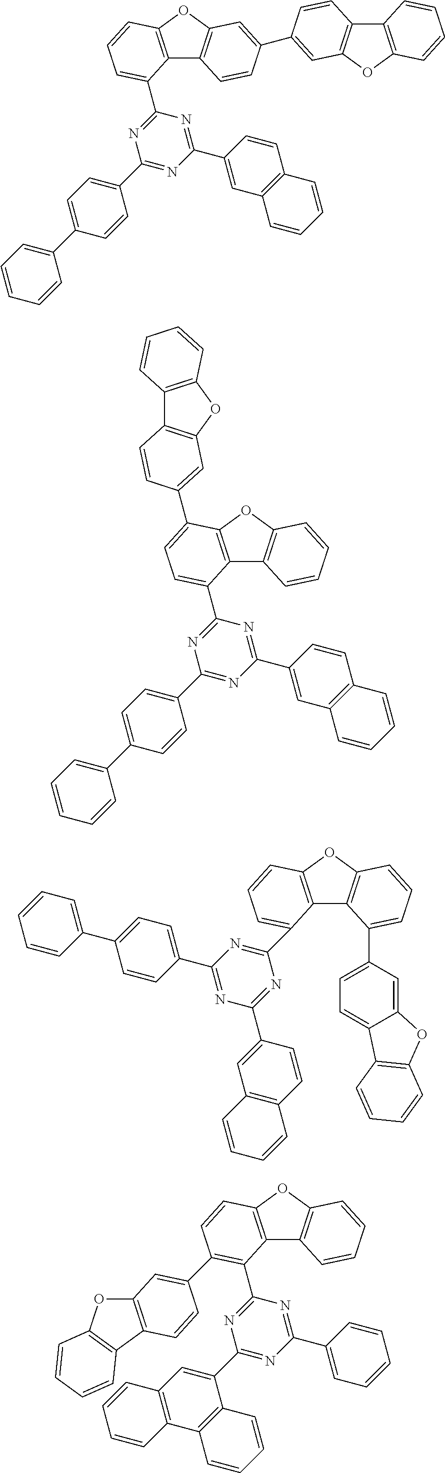

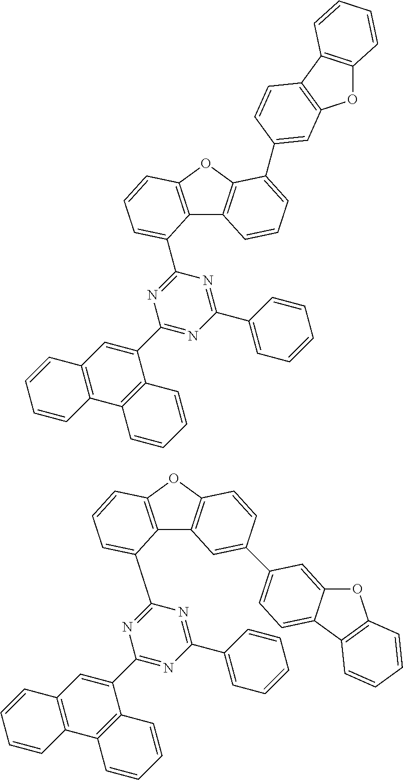

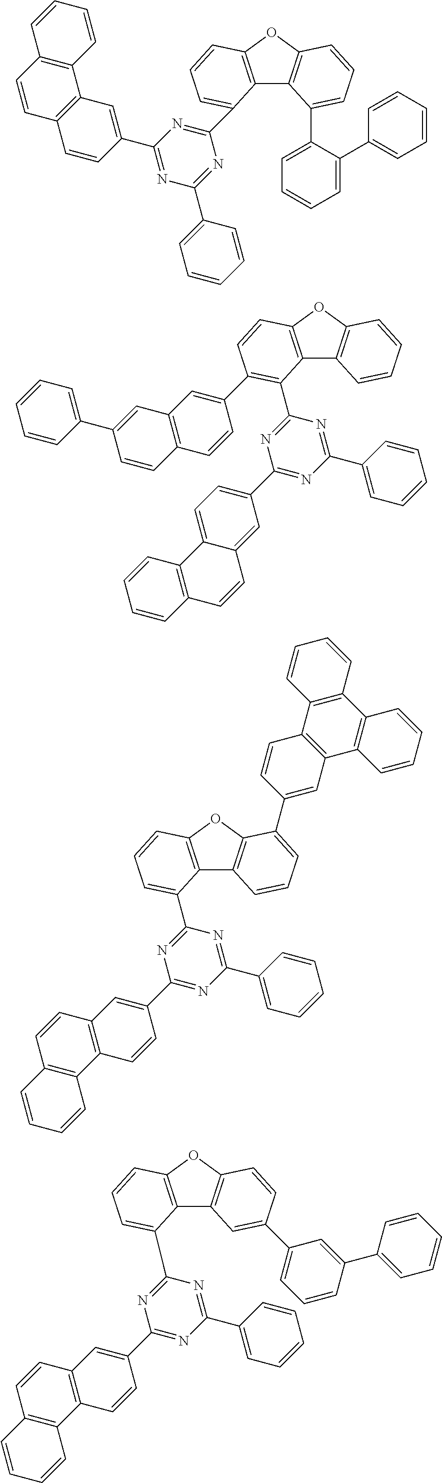 [SMILES: c1ccc(-c2nc(-c3ccc4ccc5ccccc5c4c3)nc(-c3cccc4oc5cccc(-c6ccccc6-c6ccccc6)c5c34)n2)cc1 ; c1ccc(-c2ccc3ccc(-c4ccc5oc6ccccc6c5c4-c4nc(-c5ccccc5)nc(-c5ccc6c(ccc7ccccc76)c5)n4)cc3c2)cc1 ; c1ccc(-c2nc(-c3ccc4c(ccc5ccccc54)c3)nc(-c3cccc4oc5c(-c6ccc7c8ccccc8c8ccccc8c7c6)cccc5c34)n2)cc1 ; c1ccc(-c2cccc(-c3ccc4oc5cccc(-c6nc(-c7ccccc7)nc(-c7ccc8c(ccc9ccccc98)c7)n6)c5c4c3)c2)cc1]

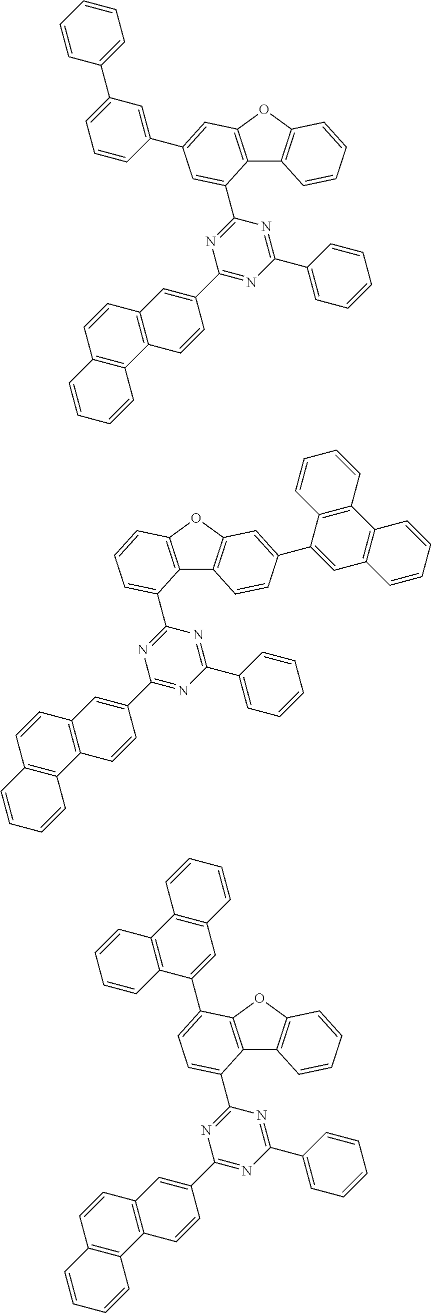 [SMILES: c1ccc(-c2cccc(-c3cc(-c4nc(-c5ccccc5)nc(-c5ccc6c(ccc7ccccc76)c5)n4)c4c(c3)oc3ccccc34)c2)cc1 ; c1ccc(-c2nc(-c3ccc4c(ccc5ccccc54)c3)nc(-c3cccc4oc5cc(-c6cc7ccccc7c7ccccc67)ccc5c34)n2)cc1 ; c1ccc(-c2nc(-c3ccc4c(ccc5ccccc54)c3)nc(-c3ccc(-c4cc5ccccc5c5ccccc45)c4oc5ccccc5c34)n2)cc1]

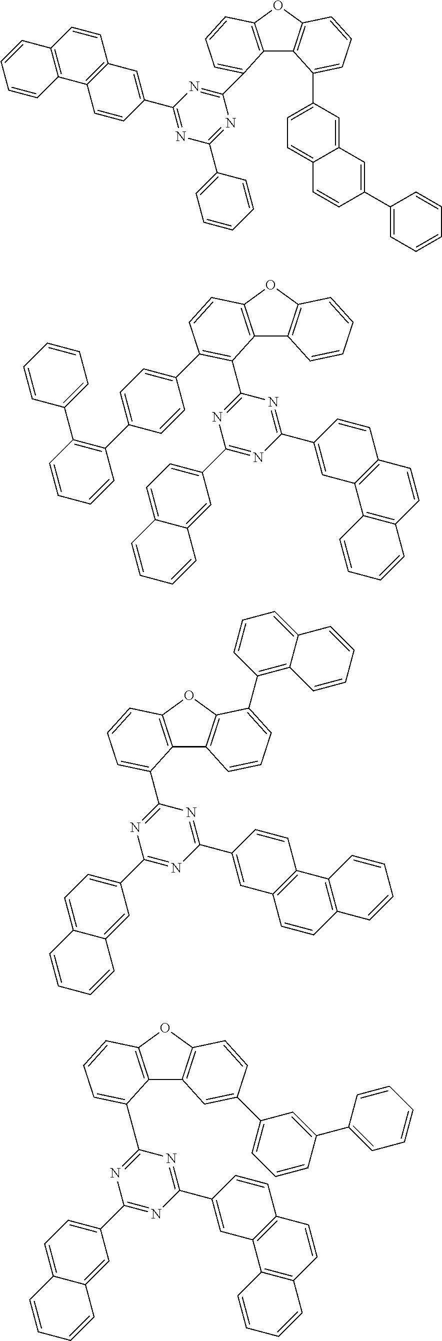 [SMILES: c1ccc(-c2ccc3ccc(-c4cccc5oc6cccc(-c7nc(-c8ccccc8)nc(-c8ccc9c(ccc%10ccccc%109)c8)n7)c6c45)cc3c2)cc1 ; c1ccc(-c2ccccc2-c2ccc(-c3ccc4oc5ccccc5c4c3-c3nc(-c4ccc5ccccc5c4)nc(-c4ccc5ccc6ccccc6c5c4)n3)cc2)cc1 ; c1ccc2cc(-c3nc(-c4ccc5c(ccc6ccccc65)c4)nc(-c4cccc5oc6c(-c7cccc8ccccc78)cccc6c45)n3)ccc2c1 ; c1ccc(-c2cccc(-c3ccc4oc5cccc(-c6nc(-c7ccc8ccccc8c7)nc(-c7ccc8ccc9ccccc9c8c7)n6)c5c4c3)c2)cc1]

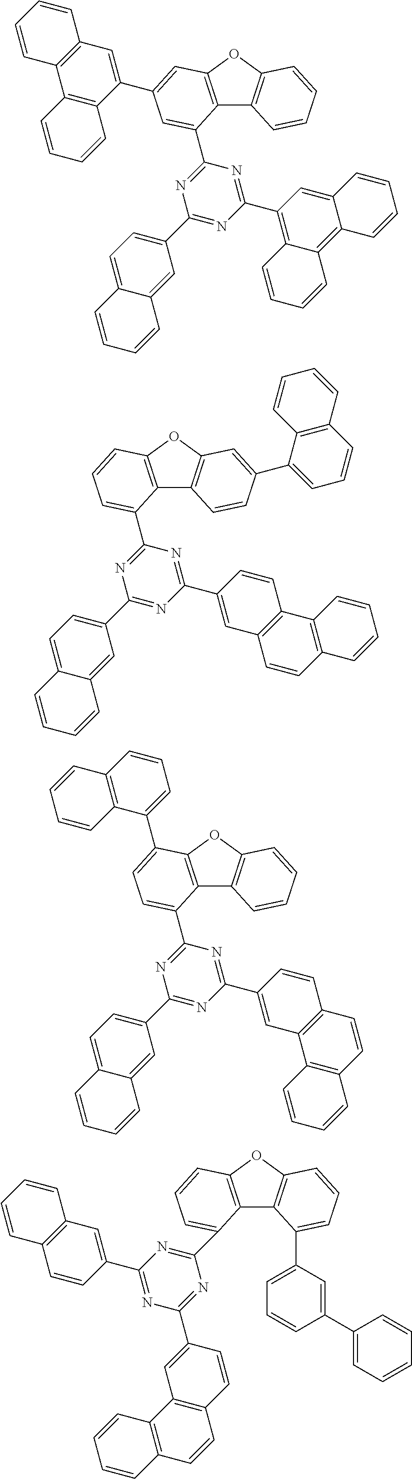

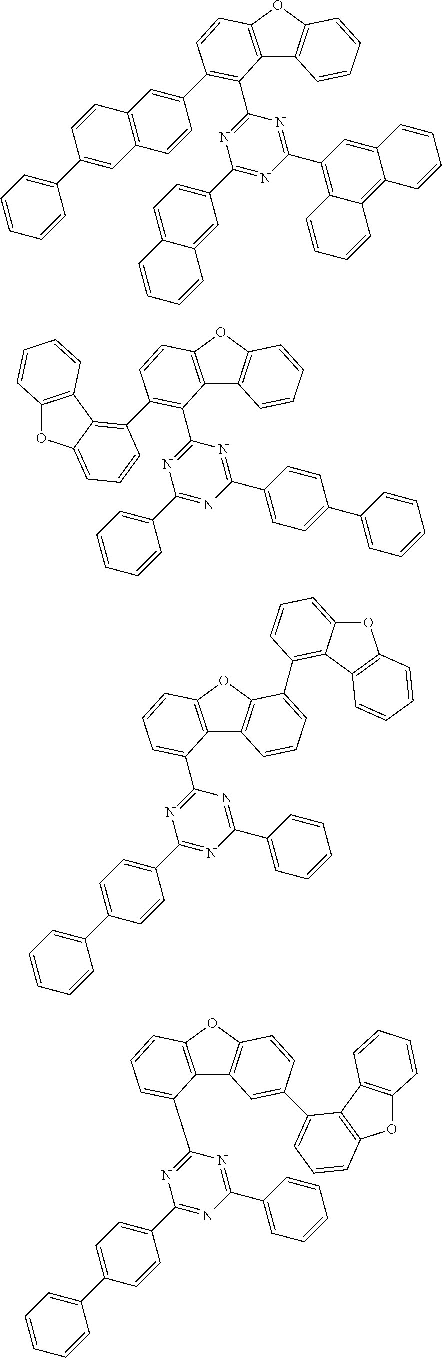

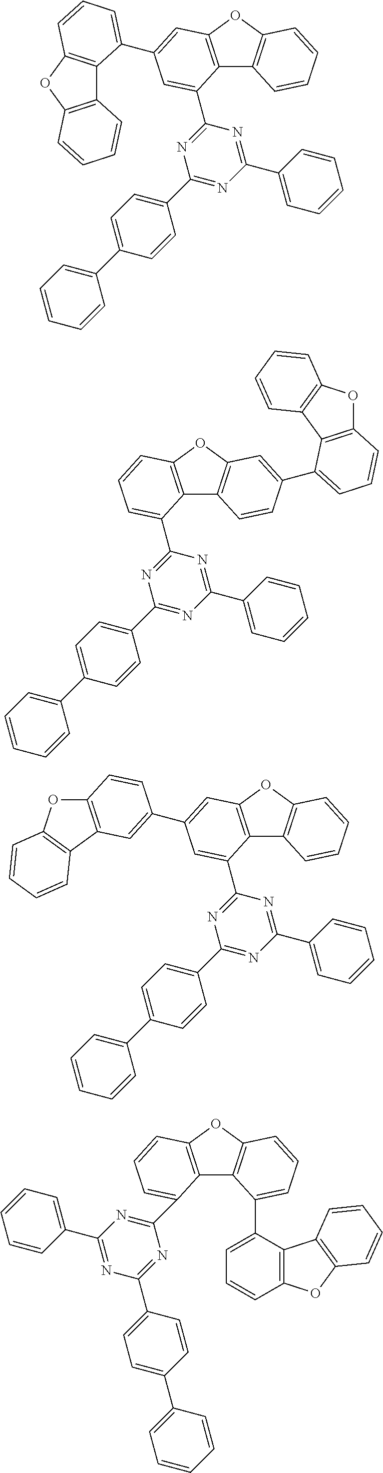

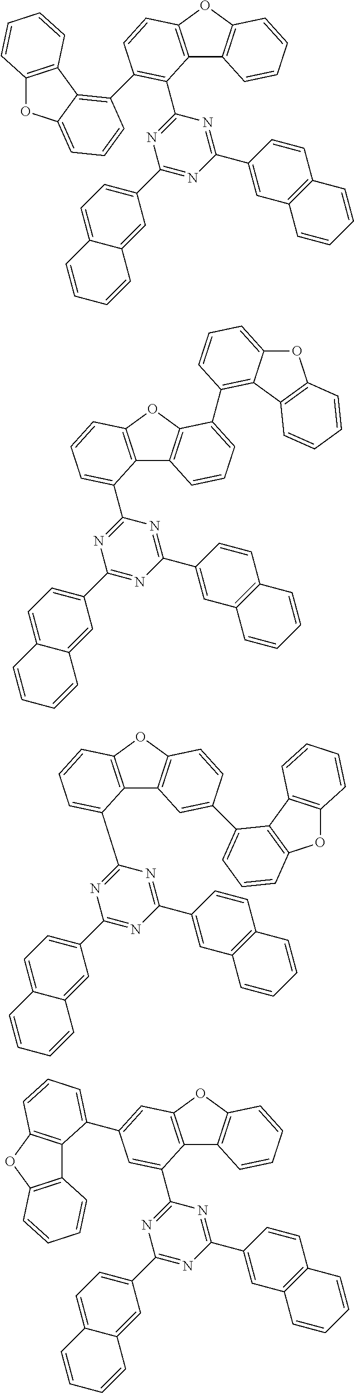

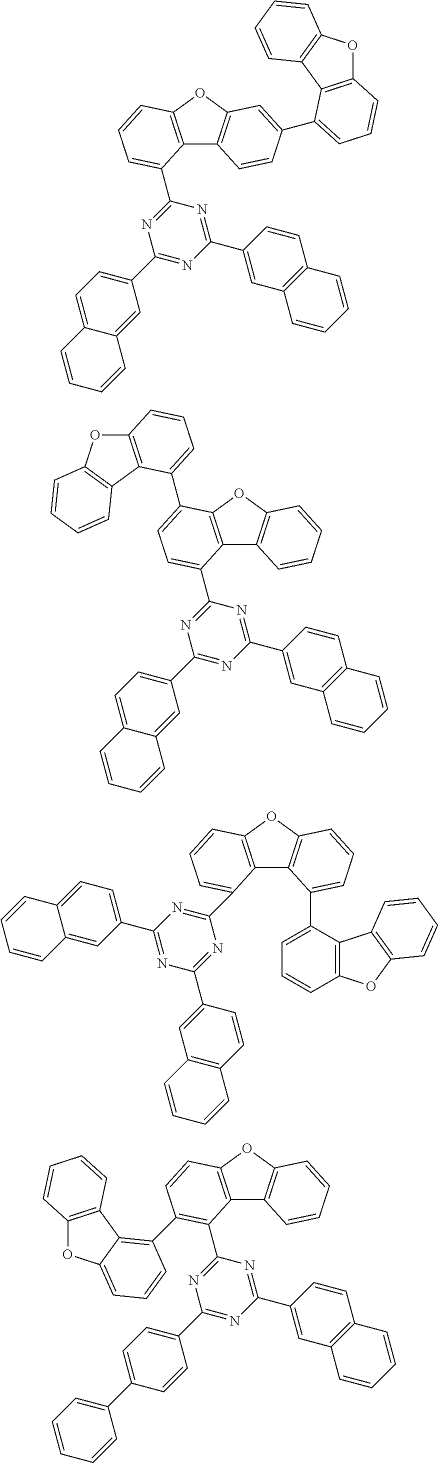

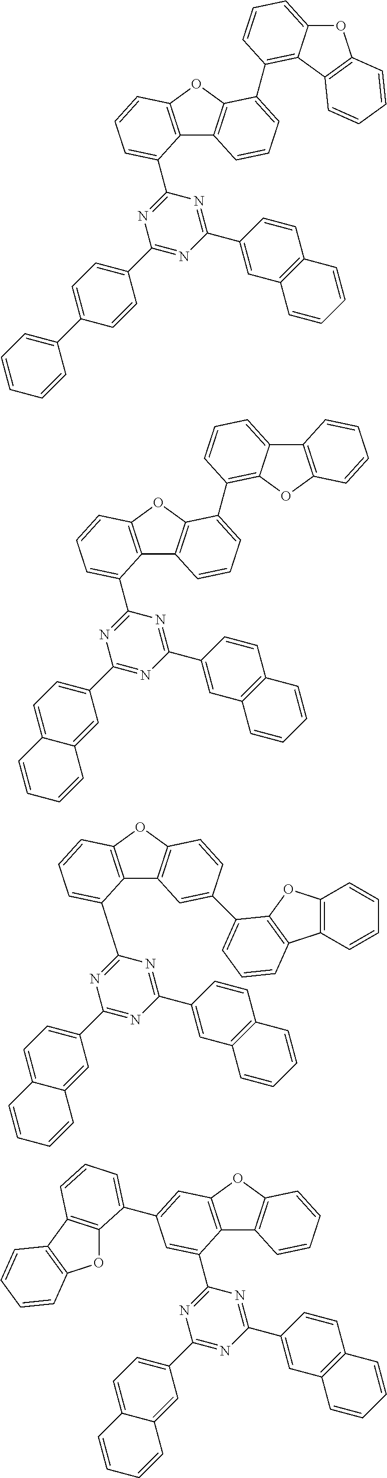

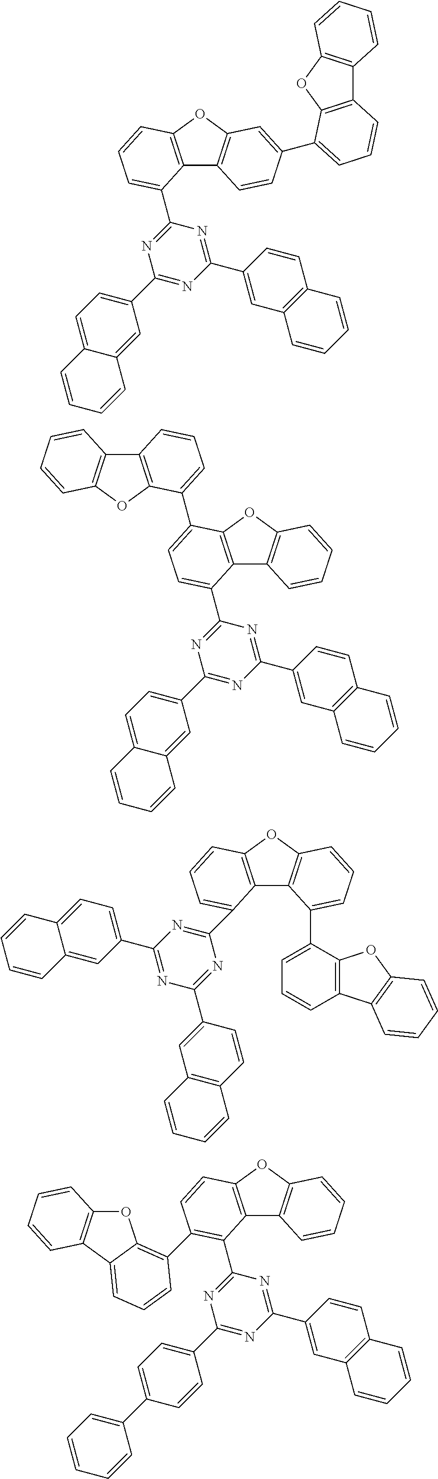

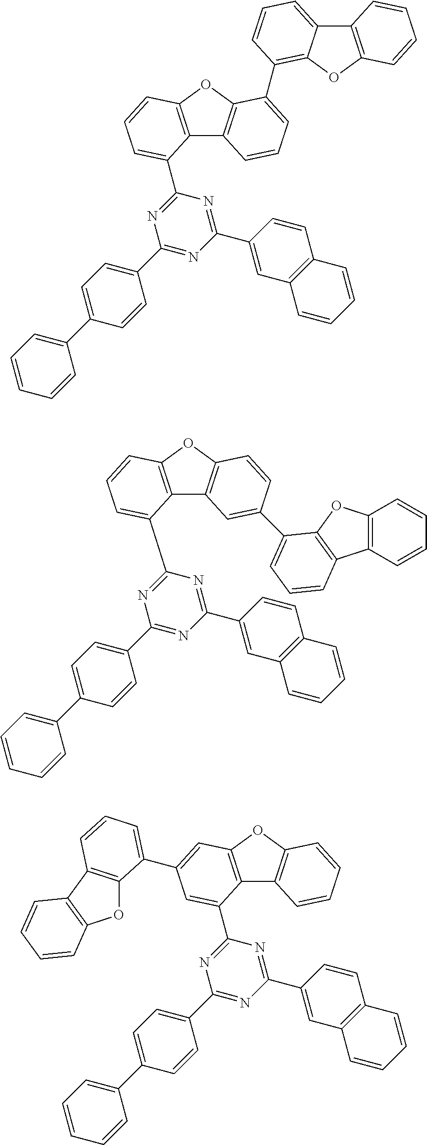 [SMILES: c1ccc(-c2ccc(-c3nc(-c4ccc5ccccc5c4)nc(-c4cccc5oc6c(-c7cccc8c7oc7ccccc78)cccc6c45)n3)cc2)cc1 ; c1ccc(-c2ccc(-c3nc(-c4ccc5ccccc5c4)nc(-c4cccc5oc6ccc(-c7cccc8c7oc7ccccc78)cc6c45)n3)cc2)cc1 ; c1ccc(-c2ccc(-c3nc(-c4ccc5ccccc5c4)nc(-c4cc(-c5cccc6c5oc5ccccc56)cc5oc6ccccc6c45)n3)cc2)cc1]

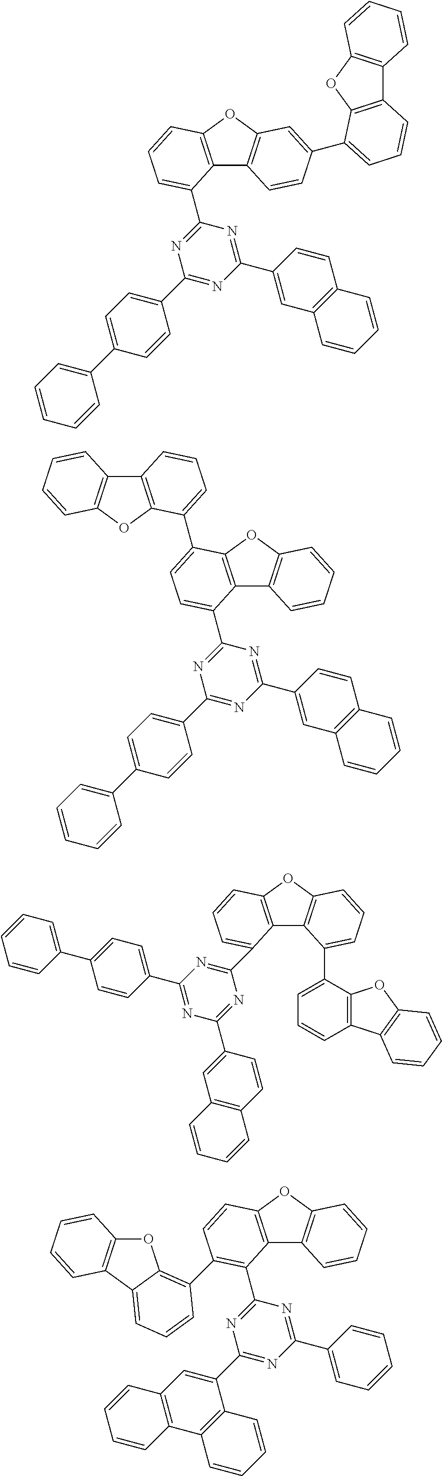

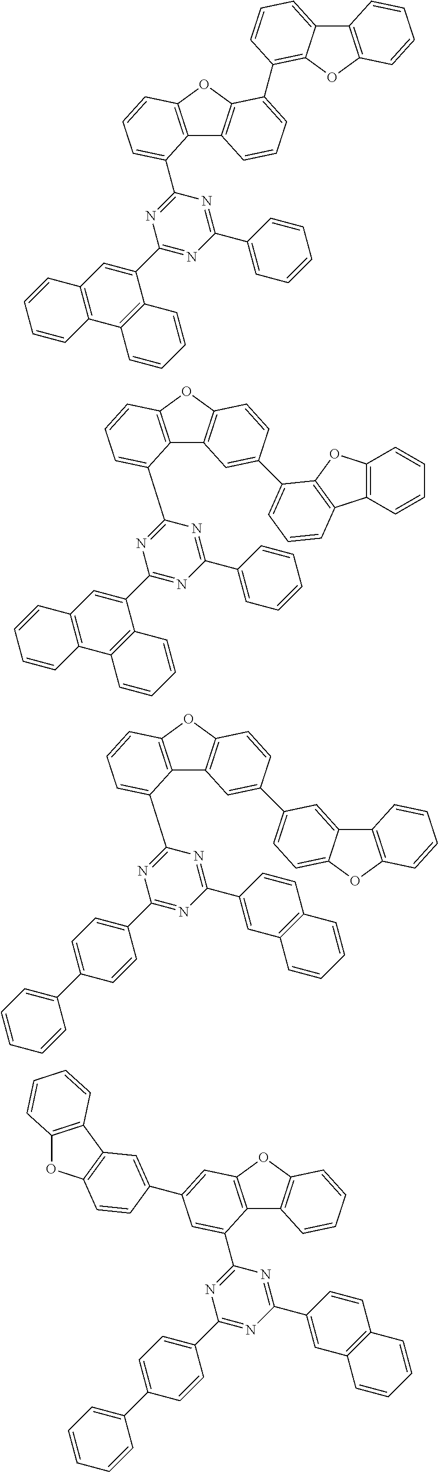

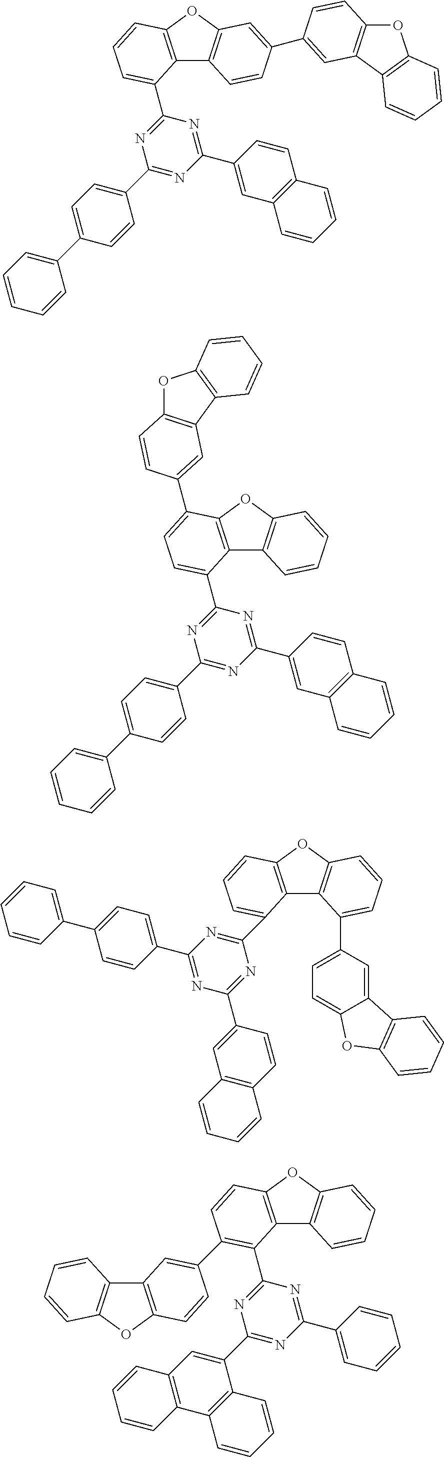

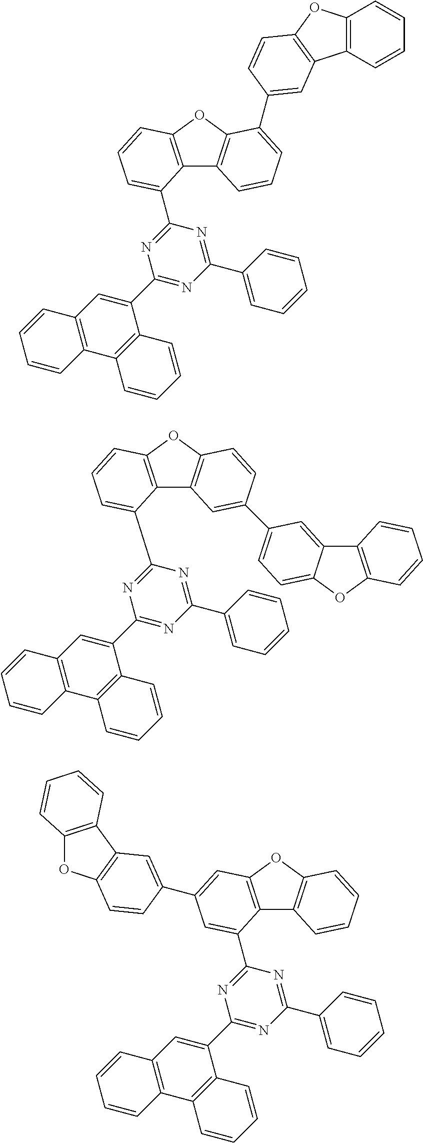

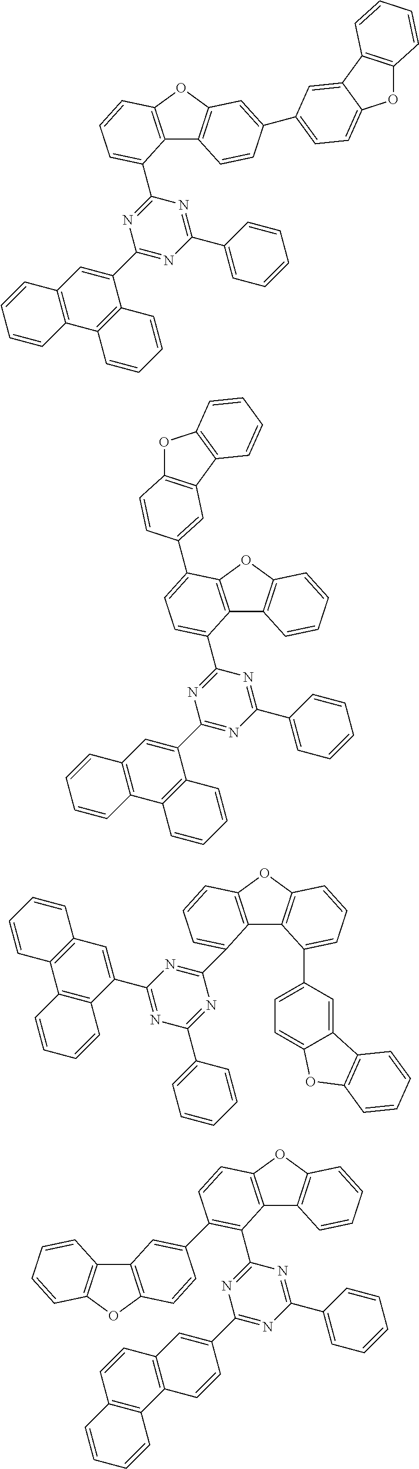

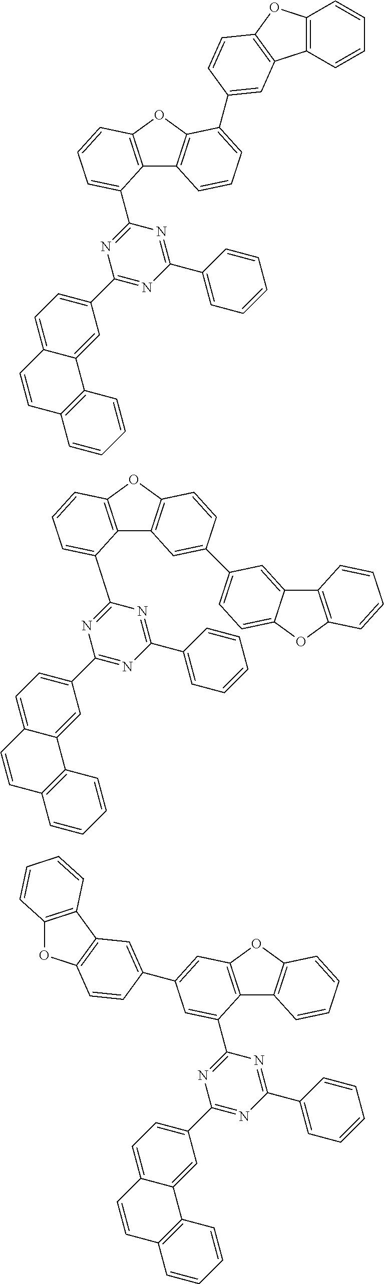

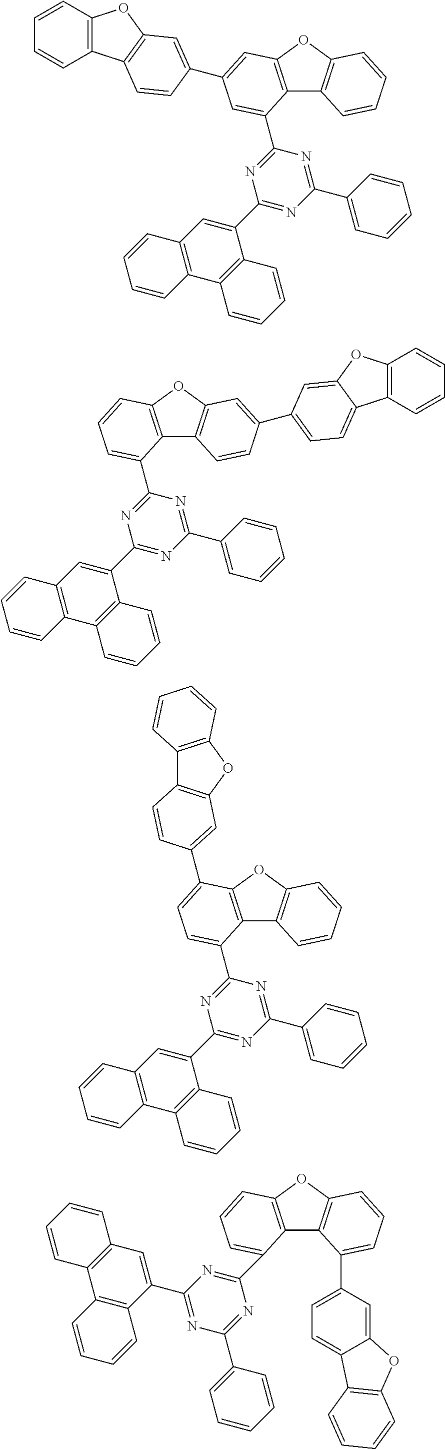

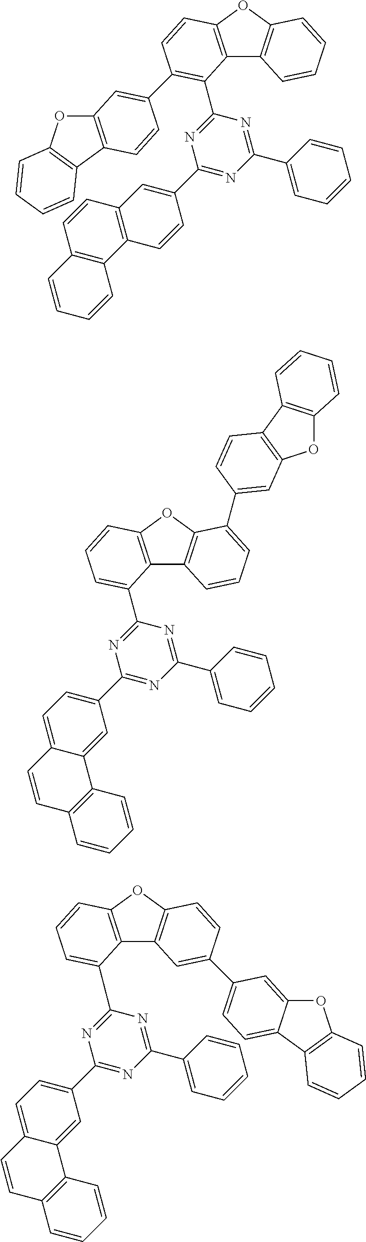 [SMILES: c1ccc(-c2nc(-c3ccc4c(ccc5ccccc54)c3)nc(-c3c(-c4ccc5c(c4)oc4ccccc45)ccc4oc5ccccc5c34)n2)cc1 ; c1ccc(-c2nc(-c3ccc4ccc5ccccc5c4c3)nc(-c3cccc4oc5c(-c6ccc7c(c6)oc6ccccc67)cccc5c34)n2)cc1 ; c1ccc(-c2nc(-c3ccc4ccc5ccccc5c4c3)nc(-c3cccc4oc5ccc(-c6ccc7c(c6)oc6ccccc67)cc5c34)n2)cc1]

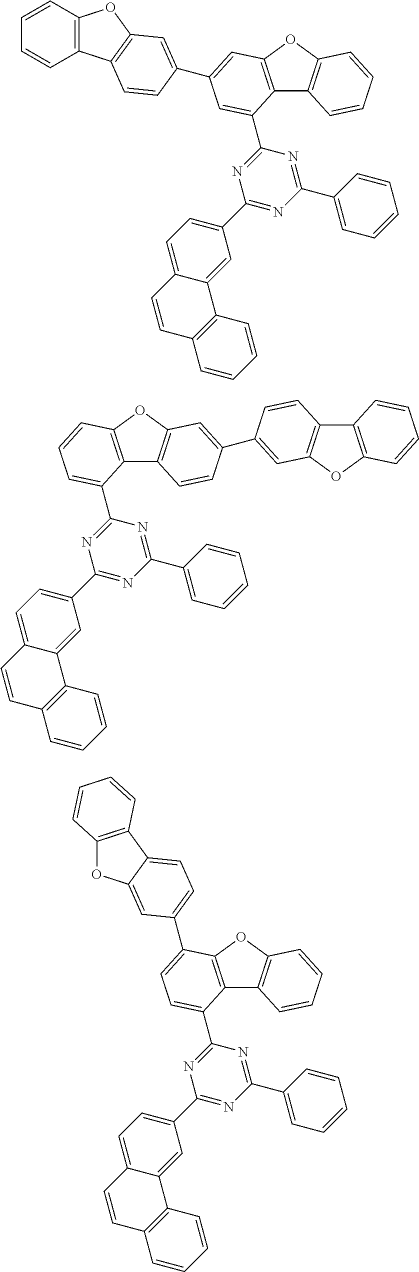

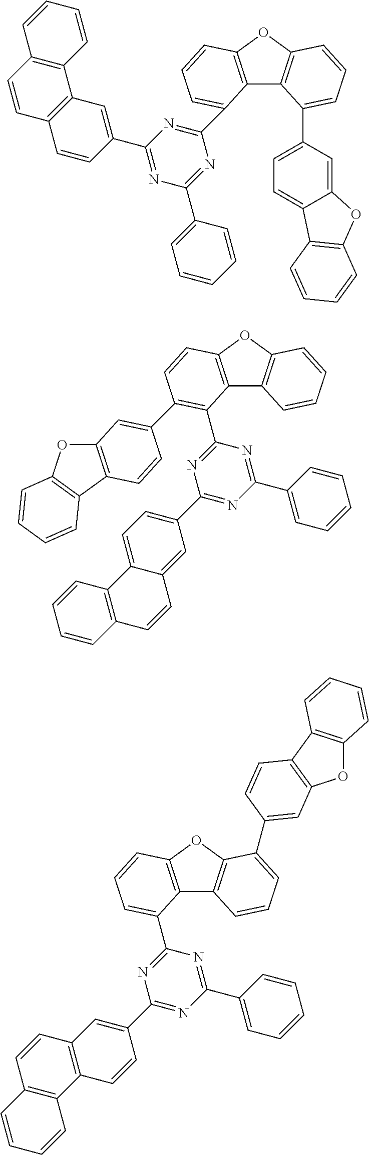 [SMILES: c1ccc(-c2nc(-c3ccc4ccc5ccccc5c4c3)nc(-c3cccc4oc5cccc(-c6ccc7c(c6)oc6ccccc67)c5c34)n2)cc1 ; c1ccc(-c2nc(-c3ccc4c(ccc5ccccc54)c3)nc(-c3c(-c4ccc5c(c4)oc4ccccc45)ccc4oc5ccccc5c34)n2)cc1 ; c1ccc(-c2nc(-c3ccc4c(ccc5ccccc54)c3)nc(-c3cccc4oc5c(-c6ccc7c(c6)oc6ccccc67)cccc5c34)n2)cc1]

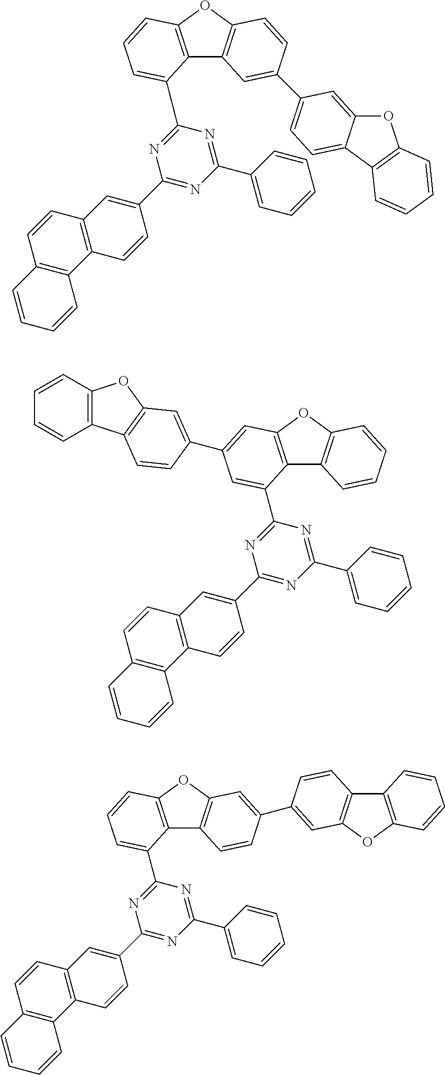

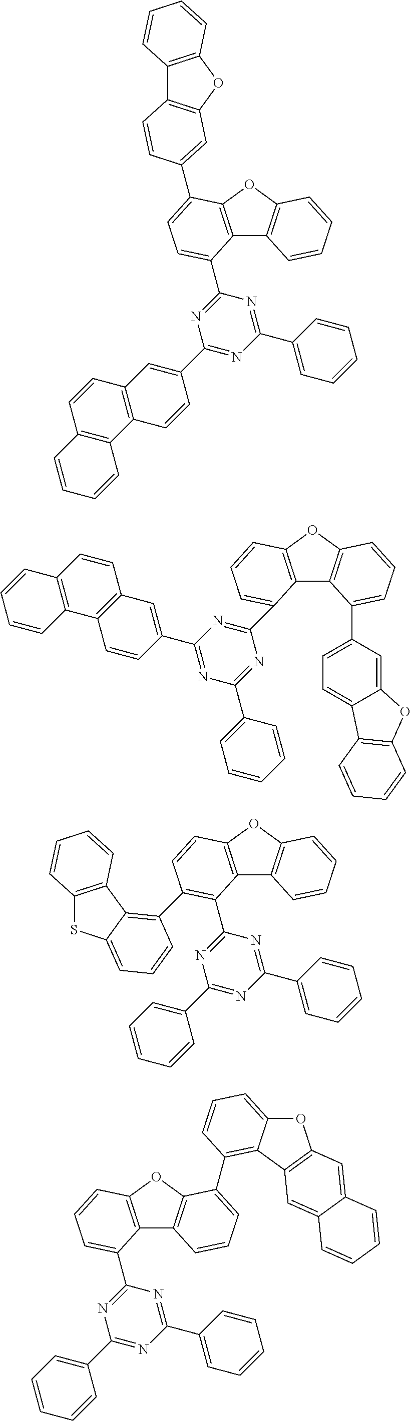

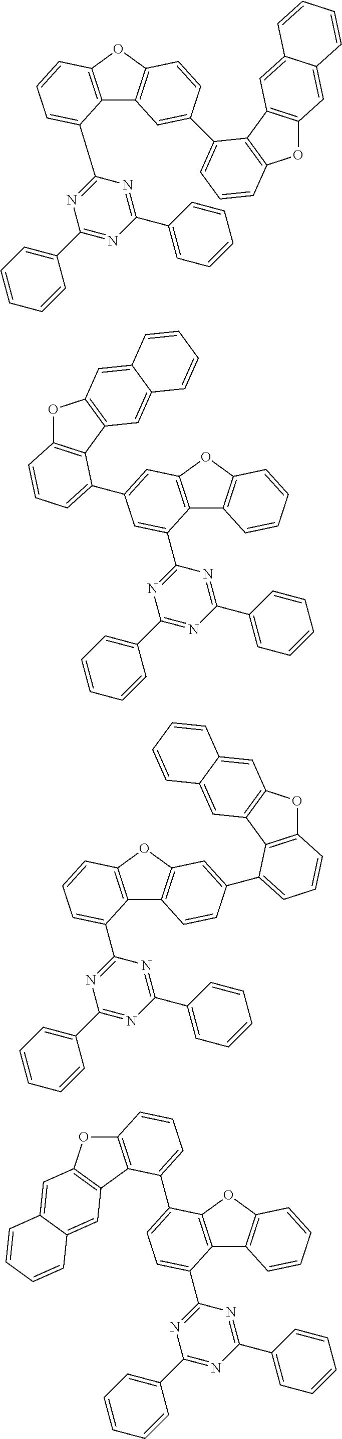 [SMILES: c1ccc(-c2nc(-c3ccccc3)nc(-c3cccc4oc5ccc(-c6cccc7oc8cc9ccccc9cc8c67)cc5c34)n2)cc1 ; c1ccc(-c2nc(-c3ccccc3)nc(-c3cc(-c4cccc5oc6cc7ccccc7cc6c45)cc4oc5ccccc5c34)n2)cc1 ; c1ccc(-c2nc(-c3ccccc3)nc(-c3cccc4oc5cc(-c6cccc7oc8cc9ccccc9cc8c67)ccc5c34)n2)cc1 ; c1ccc(-c2nc(-c3ccccc3)nc(-c3ccc(-c4cccc5oc6cc7ccccc7cc6c45)c4oc5ccccc5c34)n2)cc1]

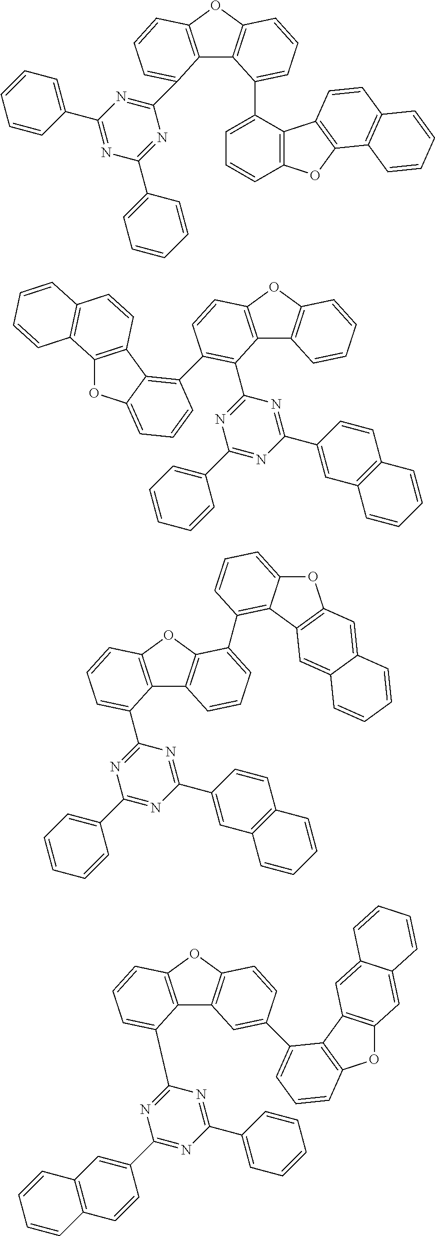

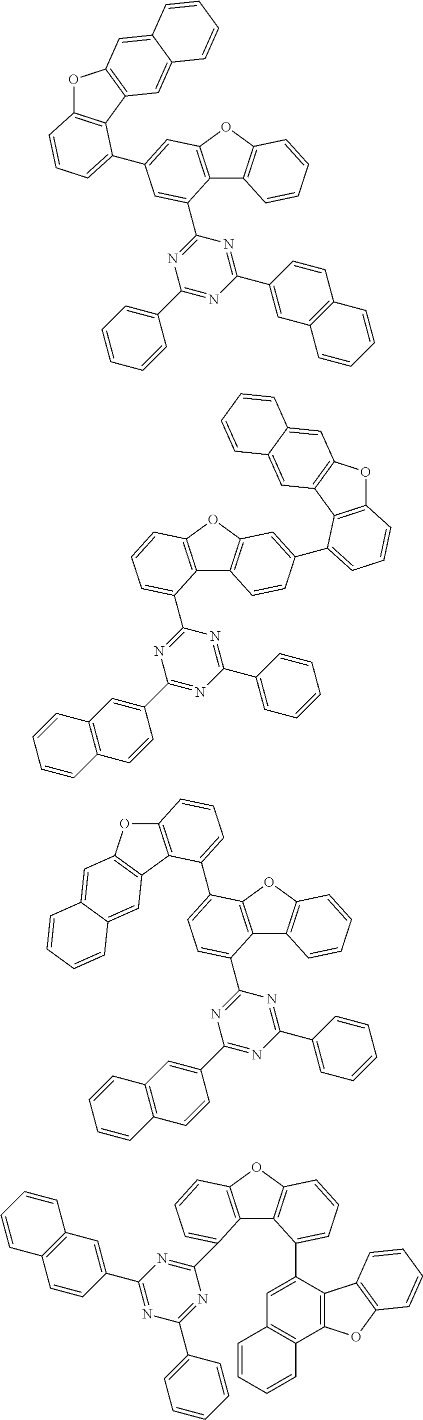 [SMILES: c1ccc(-c2nc(-c3ccc4ccccc4c3)nc(-c3cc(-c4cccc5oc6cc7ccccc7cc6c45)cc4oc5ccccc5c34)n2)cc1 ; c1ccc(-c2nc(-c3ccc4ccccc4c3)nc(-c3cccc4oc5cc(-c6cccc7oc8cc9ccccc9cc8c67)ccc5c34)n2)cc1 ; c1ccc(-c2nc(-c3ccc4ccccc4c3)nc(-c3ccc(-c4cccc5oc6cc7ccccc7cc6c45)c4oc5ccccc5c34)n2)cc1 ; c1ccc(-c2nc(-c3ccc4ccccc4c3)nc(-c3cccc4oc5cccc(-c6cc7ccccc7c7oc8ccccc8c67)c5c34)n2)cc1]

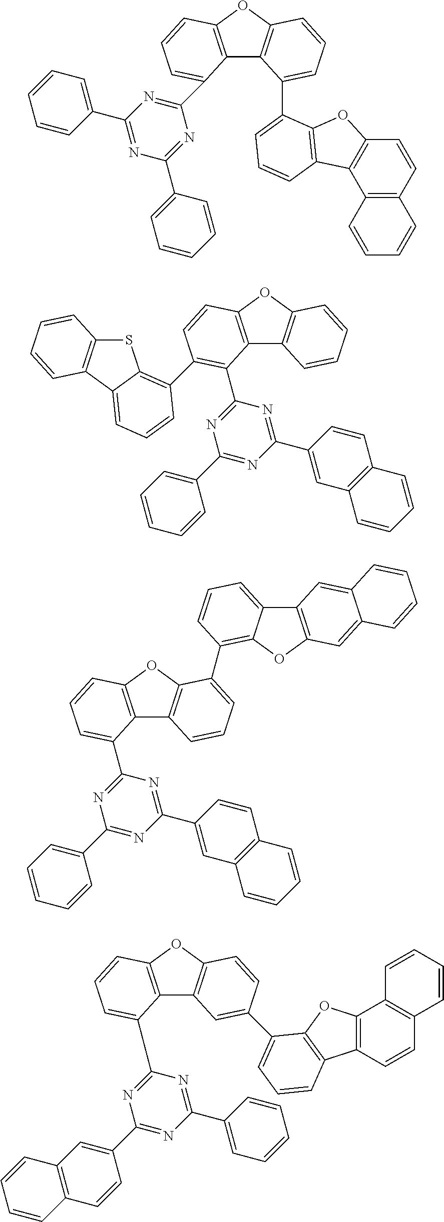

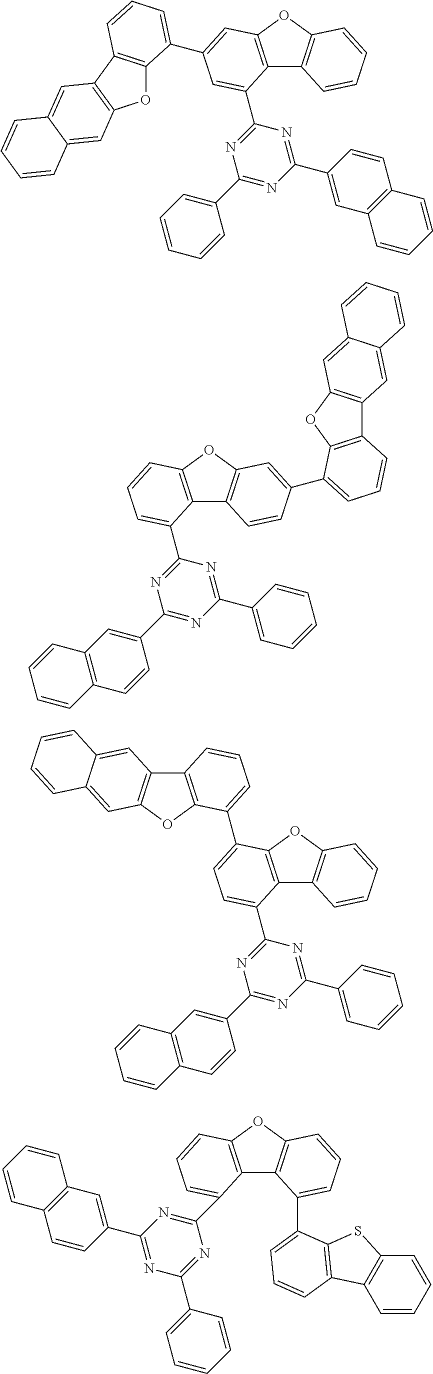

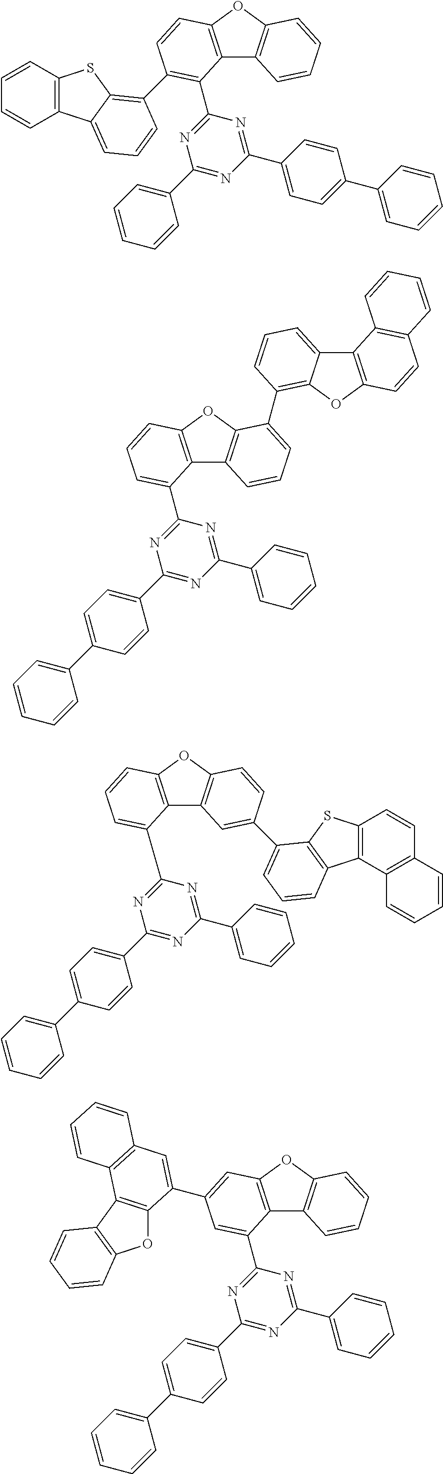 [SMILES: c1ccc(-c2ccc(-c3nc(-c4ccccc4)nc(-c4c(-c5cccc6c5sc5ccccc56)ccc5oc6ccccc6c45)n3)cc2)cc1 ; c1ccc(-c2ccc(-c3nc(-c4ccccc4)nc(-c4cccc5oc6c(-c7cccc8c7oc7ccc9ccccc9c78)cccc6c45)n3)cc2)cc1 ; c1ccc(-c2ccc(-c3nc(-c4ccccc4)nc(-c4cccc5oc6ccc(-c7cccc8c7sc7ccc9ccccc9c78)cc6c45)n3)cc2)cc1 ; c1ccc(-c2ccc(-c3nc(-c4ccccc4)nc(-c4cc(-c5cc6ccccc6c6c5oc5ccccc56)cc5oc6ccccc6c45)n3)cc2)cc1]

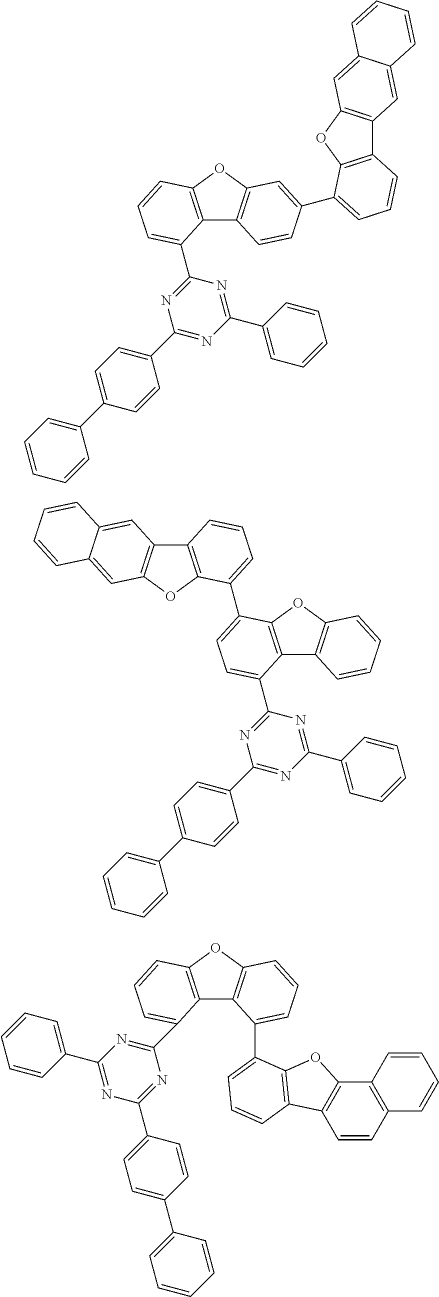

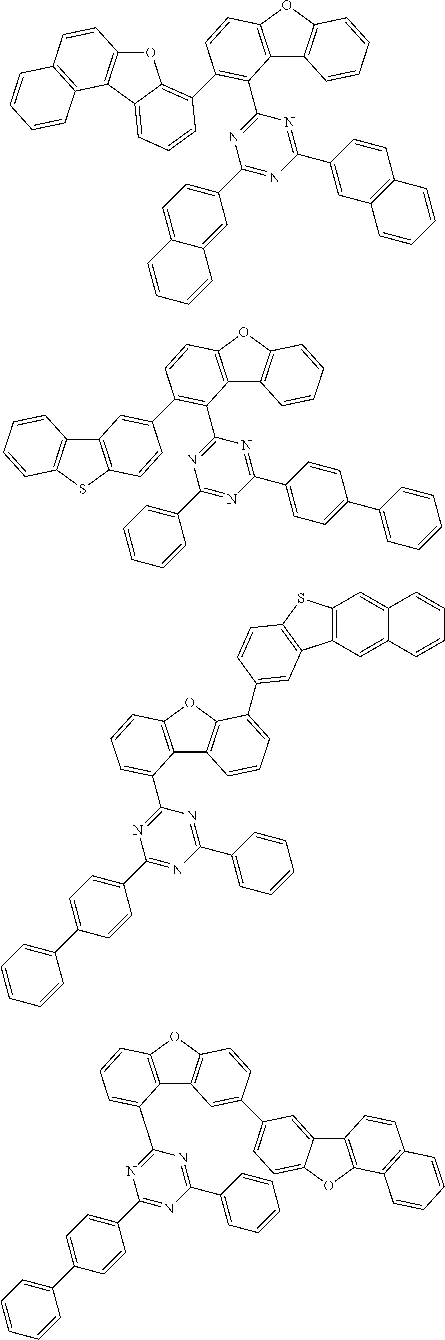

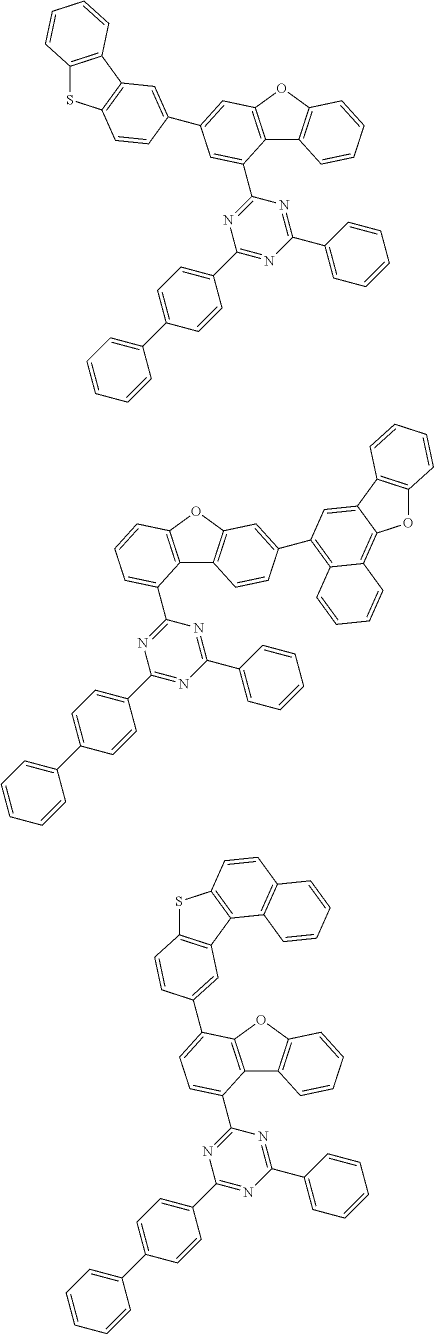 [SMILES: c1ccc(-c2ccc(-c3nc(-c4ccccc4)nc(-c4cc(-c5ccc6sc7ccccc7c6c5)cc5oc6ccccc6c45)n3)cc2)cc1 ; c1ccc(-c2ccc(-c3nc(-c4ccccc4)nc(-c4cccc5oc6cc(-c7cc8c9ccccc9oc8c8ccccc78)ccc6c45)n3)cc2)cc1 ; c1ccc(-c2ccc(-c3nc(-c4ccccc4)nc(-c4ccc(-c5ccc6sc7ccc8ccccc8c7c6c5)c5oc6ccccc6c45)n3)cc2)cc1]

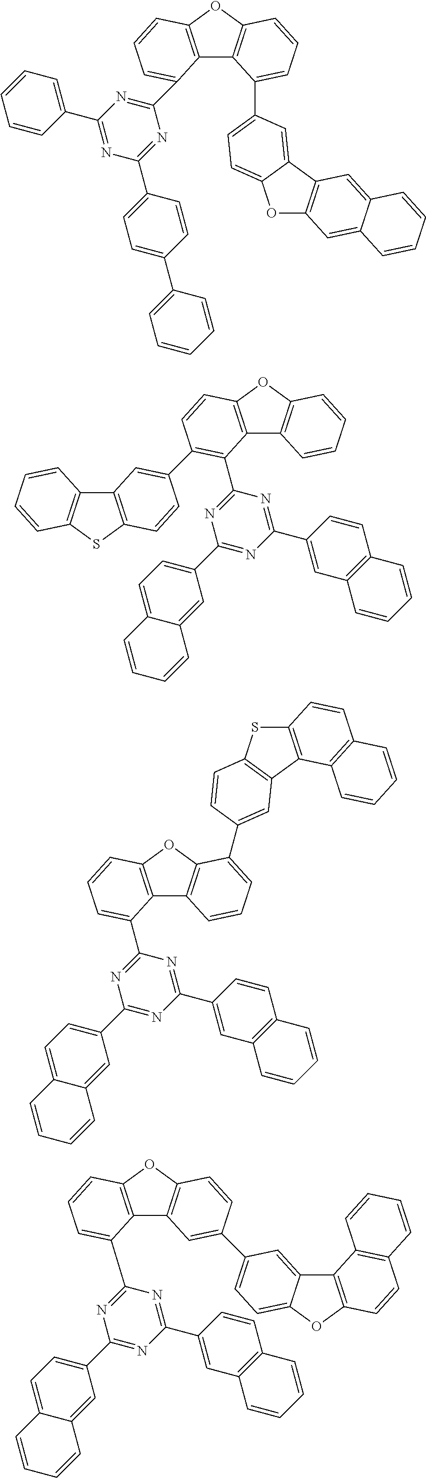

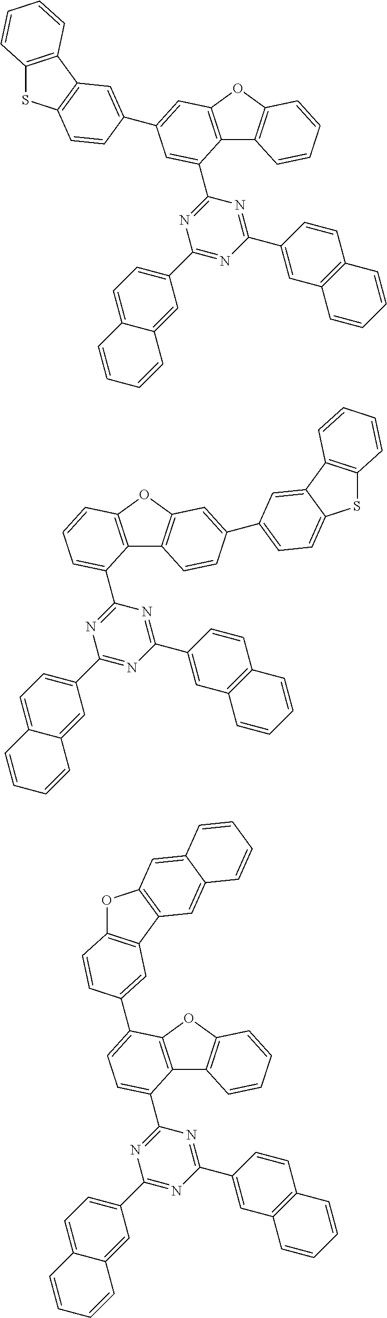 [SMILES: c1ccc2cc(-c3nc(-c4ccc5ccccc5c4)nc(-c4cc(-c5ccc6sc7ccccc7c6c5)cc5oc6ccccc6c45)n3)ccc2c1 ; c1ccc2cc(-c3nc(-c4ccc5ccccc5c4)nc(-c4cccc5oc6cc(-c7ccc8sc9ccccc9c8c7)ccc6c45)n3)ccc2c1 ; c1ccc2cc(-c3nc(-c4ccc5ccccc5c4)nc(-c4ccc(-c5ccc6oc7cc8ccccc8cc7c6c5)c5oc6ccccc6c45)n3)ccc2c1]

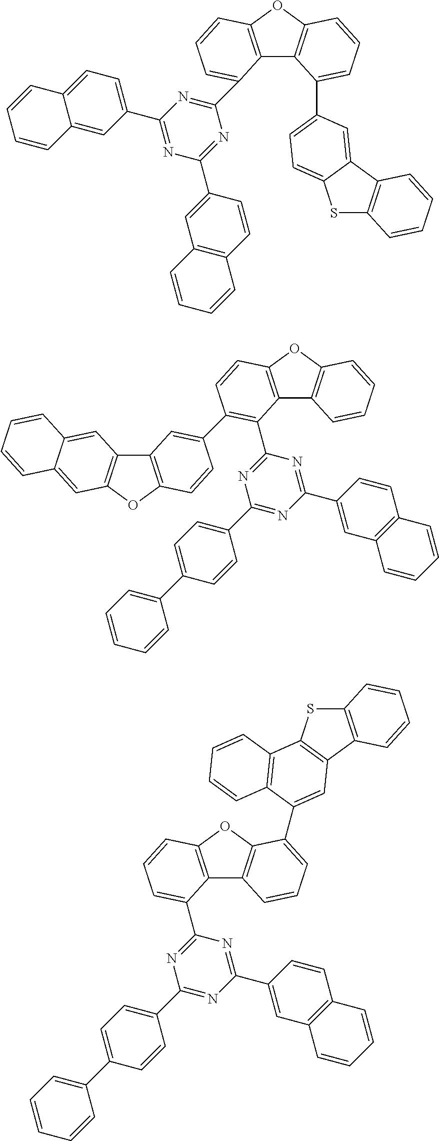 [SMILES: c1ccc2cc(-c3nc(-c4ccc5ccccc5c4)nc(-c4cccc5oc6cccc(-c7ccc8sc9ccccc9c8c7)c6c45)n3)ccc2c1 ; c1ccc(-c2ccc(-c3nc(-c4ccc5ccccc5c4)nc(-c4c(-c5ccc6oc7cc8ccccc8cc7c6c5)ccc5oc6ccccc6c45)n3)cc2)cc1 ; c1ccc(-c2ccc(-c3nc(-c4ccc5ccccc5c4)nc(-c4cccc5oc6c(-c7cc8c9ccccc9sc8c8ccccc78)cccc6c45)n3)cc2)cc1]

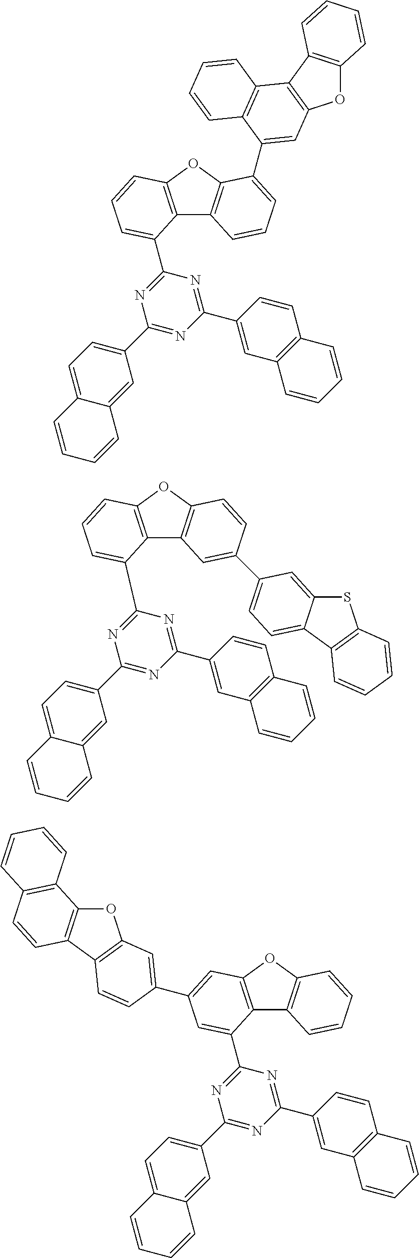

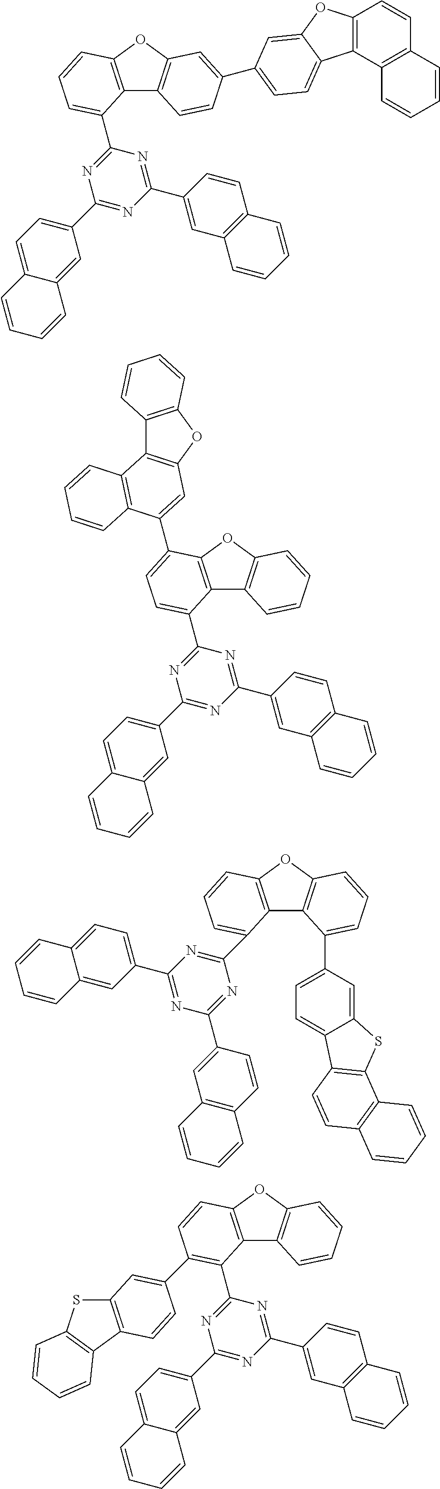

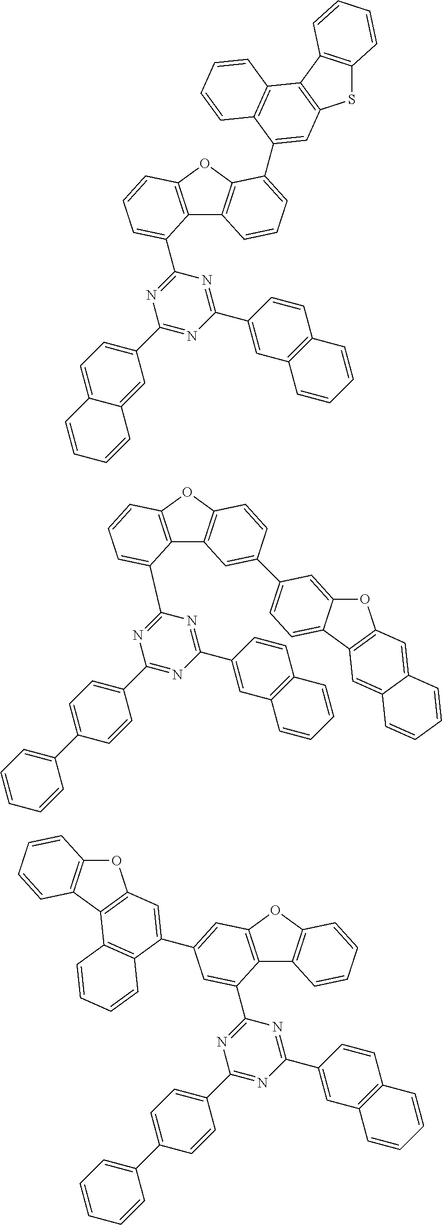

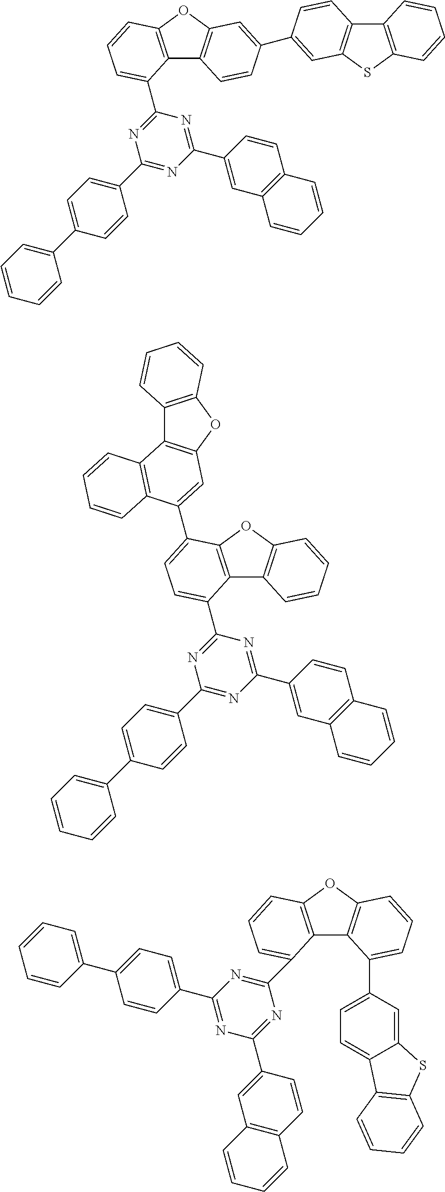 [SMILES: c1ccc(-c2ccc(-c3nc(-c4ccc5ccccc5c4)nc(-c4cccc5oc6cc(-c7ccc8c(c7)sc7ccccc78)ccc6c45)n3)cc2)cc1 ; c1ccc(-c2ccc(-c3nc(-c4ccc5ccccc5c4)nc(-c4ccc(-c5cc6oc7ccccc7c6c6ccccc56)c5oc6ccccc6c45)n3)cc2)cc1 ; c1ccc(-c2ccc(-c3nc(-c4ccc5ccccc5c4)nc(-c4cccc5oc6cccc(-c7ccc8c(c7)sc7ccccc78)c6c45)n3)cc2)cc1]

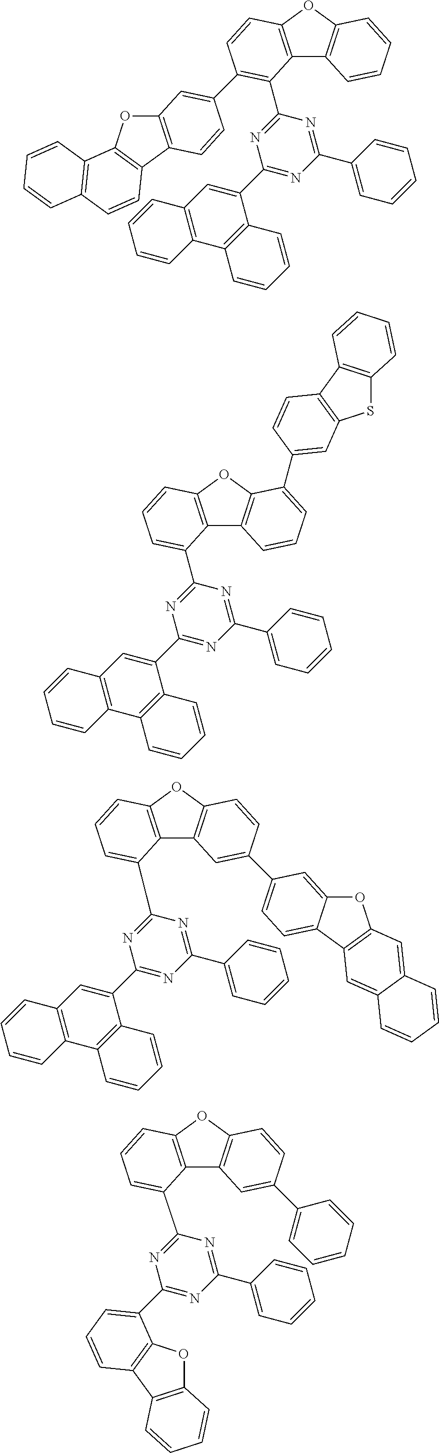

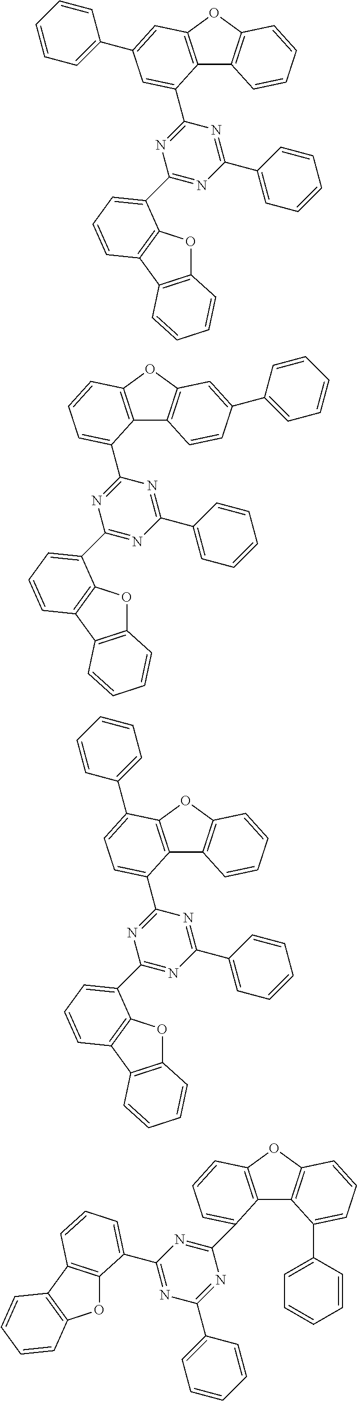

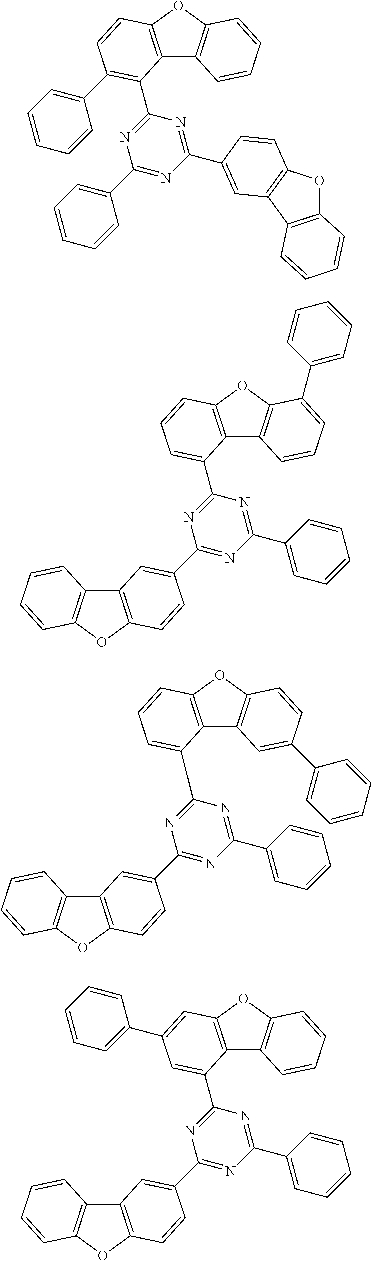

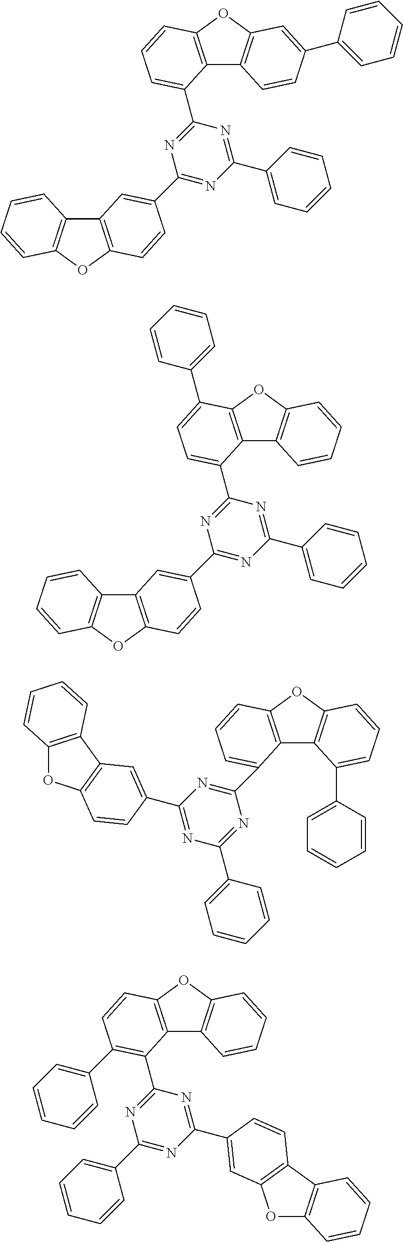 [SMILES: c1ccc(-c2ccc3c(c2)oc2cccc(-c4nc(-c5ccccc5)nc(-c5ccc6oc7ccccc7c6c5)n4)c23)cc1 ; c1ccc(-c2nc(-c3ccc4oc5ccccc5c4c3)nc(-c3ccc(-c4ccccc4)c4oc5ccccc5c34)n2)cc1 ; c1ccc(-c2nc(-c3ccc4oc5ccccc5c4c3)nc(-c3cccc4oc5cccc(-c6ccccc6)c5c34)n2)cc1 ; c1ccc(-c2nc(-c3ccc4c(c3)oc3ccccc34)nc(-c3c(-c4ccccc4)ccc4oc5ccccc5c34)n2)cc1]

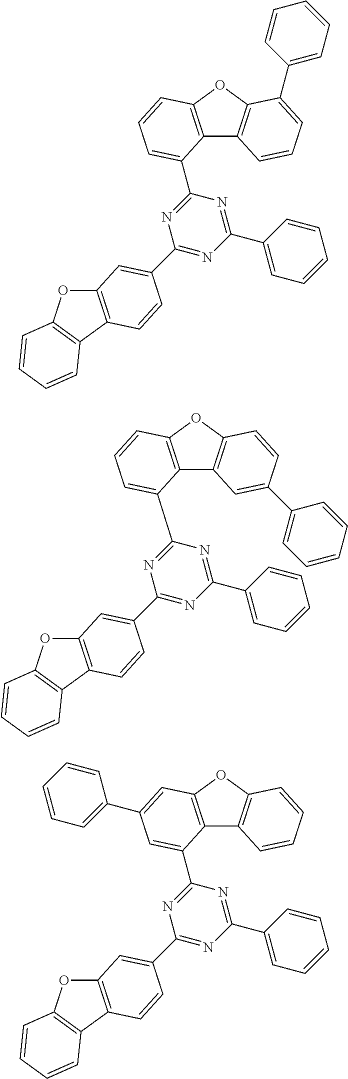

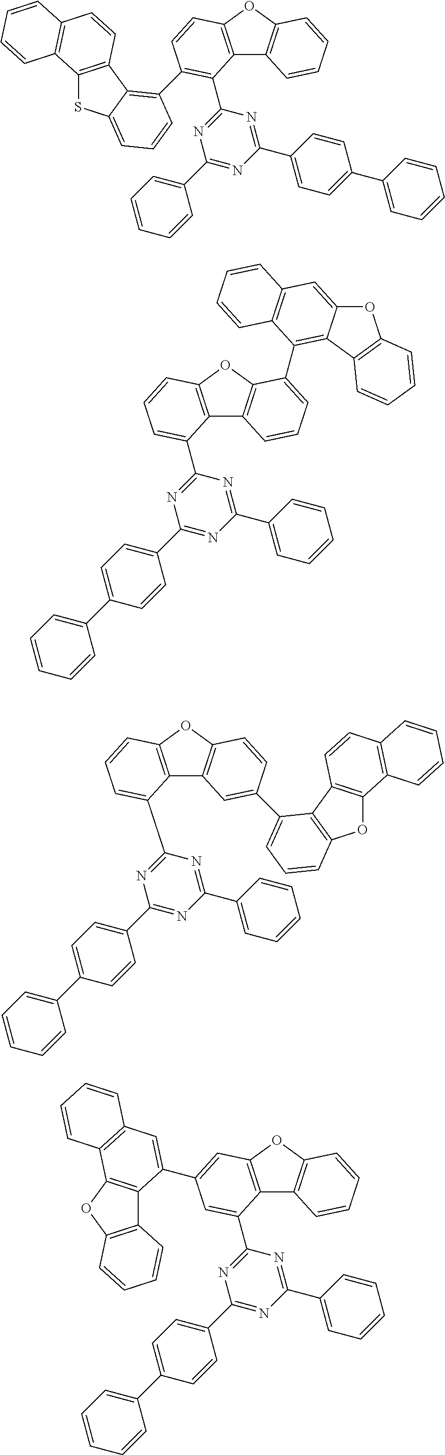 [SMILES: c1ccc(-c2ccc(-c3nc(-c4ccccc4)nc(-c4c(-c5cccc6sc7c8ccccc8ccc7c56)ccc5oc6ccccc6c45)n3)cc2)cc1 ; c1ccc(-c2ccc(-c3nc(-c4ccccc4)nc(-c4cccc5oc6c(-c7c8ccccc8cc8oc9ccccc9c78)cccc6c45)n3)cc2)cc1 ; c1ccc(-c2ccc(-c3nc(-c4ccccc4)nc(-c4cccc5oc6ccc(-c7cccc8oc9c%10ccccc%10ccc9c78)cc6c45)n3)cc2)cc1 ; c1ccc(-c2ccc(-c3nc(-c4ccccc4)nc(-c4cc(-c5cc6ccccc6c6oc7ccccc7c56)cc5oc6ccccc6c45)n3)cc2)cc1]

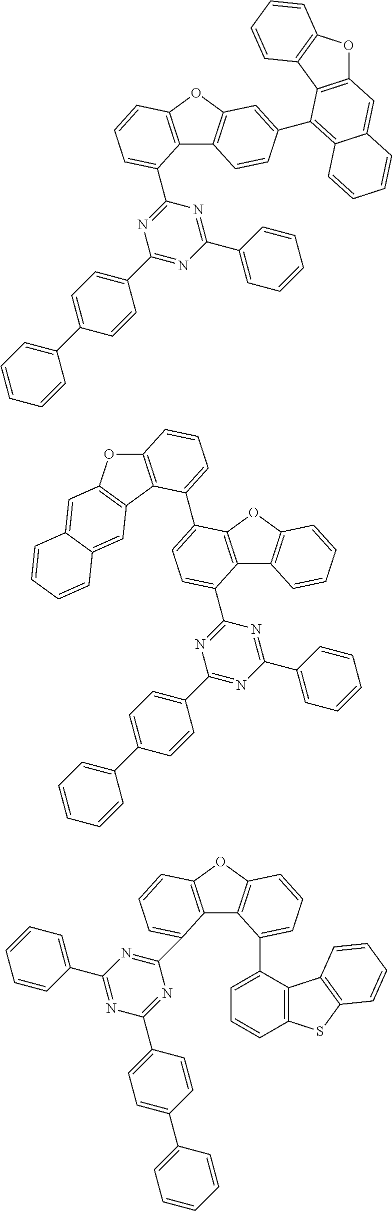 [SMILES: c1ccc(-c2ccc(-c3nc(-c4ccccc4)nc(-c4cccc5oc6cc(-c7c8ccccc8cc8oc9ccccc9c78)ccc6c45)n3)cc2)cc1 ; c1ccc(-c2ccc(-c3nc(-c4ccccc4)nc(-c4ccc(-c5cccc6oc7cc8ccccc8cc7c56)c5oc6ccccc6c45)n3)cc2)cc1 ; c1ccc(-c2ccc(-c3nc(-c4ccccc4)nc(-c4cccc5oc6cccc(-c7cccc8sc9ccccc9c78)c6c45)n3)cc2)cc1]

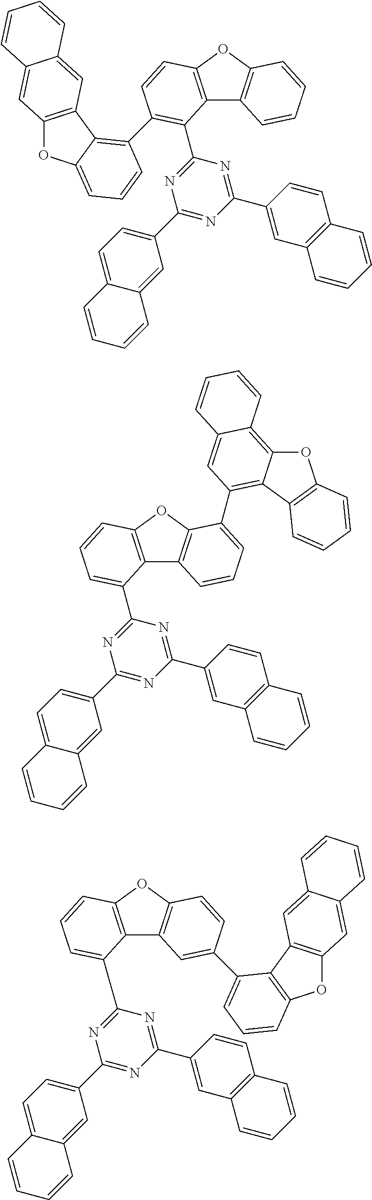 [SMILES: c1ccc2cc(-c3nc(-c4ccc5ccccc5c4)nc(-c4c(-c5cccc6oc7cc8ccccc8cc7c56)ccc5oc6ccccc6c45)n3)ccc2c1 ; c1ccc2cc(-c3nc(-c4ccc5ccccc5c4)nc(-c4cccc5oc6c(-c7cc8ccccc8c8oc9ccccc9c78)cccc6c45)n3)ccc2c1 ; c1ccc2cc(-c3nc(-c4ccc5ccccc5c4)nc(-c4cccc5oc6ccc(-c7cccc8oc9cc%10ccccc%10cc9c78)cc6c45)n3)ccc2c1]

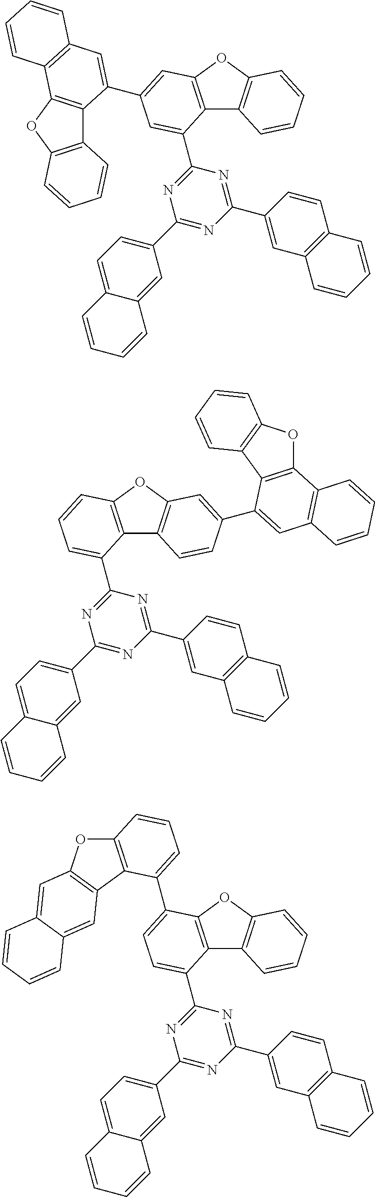 [SMILES: c1ccc2cc(-c3nc(-c4ccc5ccccc5c4)nc(-c4cc(-c5cc6ccccc6c6oc7ccccc7c56)cc5oc6ccccc6c45)n3)ccc2c1 ; c1ccc2cc(-c3nc(-c4ccc5ccccc5c4)nc(-c4cccc5oc6cc(-c7cc8ccccc8c8oc9ccccc9c78)ccc6c45)n3)ccc2c1 ; c1ccc2cc(-c3nc(-c4ccc5ccccc5c4)nc(-c4ccc(-c5cccc6oc7cc8ccccc8cc7c56)c5oc6ccccc6c45)n3)ccc2c1]

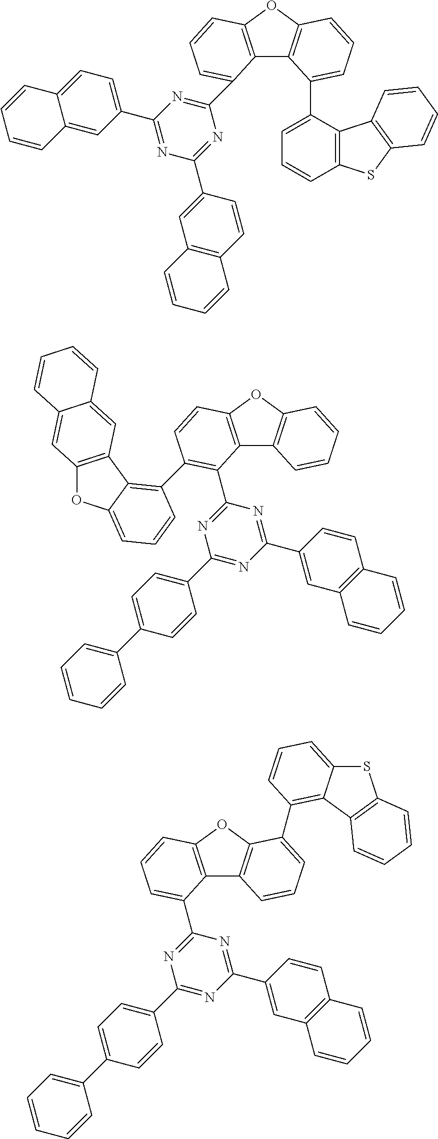 [SMILES: c1ccc2cc(-c3nc(-c4ccc5ccccc5c4)nc(-c4cccc5oc6cccc(-c7cccc8sc9ccccc9c78)c6c45)n3)ccc2c1 ; c1ccc(-c2ccc(-c3nc(-c4ccc5ccccc5c4)nc(-c4c(-c5cccc6oc7cc8ccccc8cc7c56)ccc5oc6ccccc6c45)n3)cc2)cc1 ; c1ccc(-c2ccc(-c3nc(-c4ccc5ccccc5c4)nc(-c4cccc5oc6c(-c7cccc8sc9ccccc9c78)cccc6c45)n3)cc2)cc1]

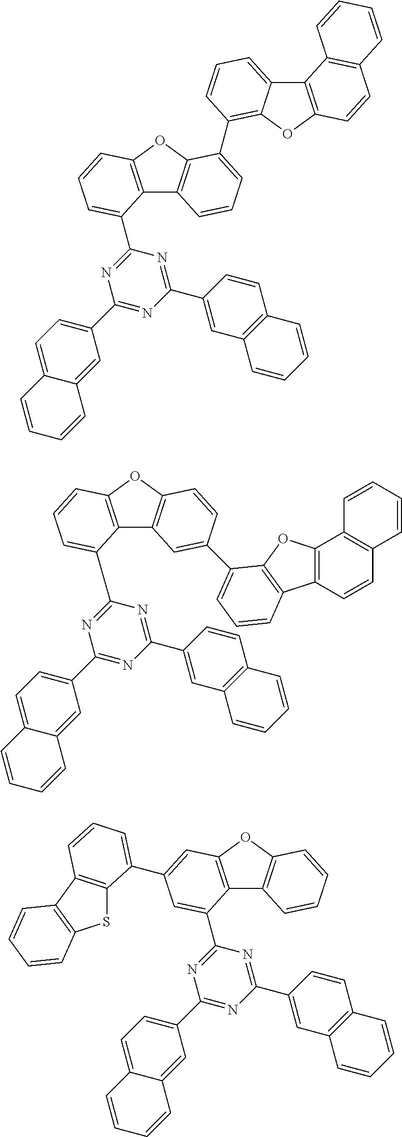 [SMILES: c1ccc2cc(-c3nc(-c4ccc5ccccc5c4)nc(-c4cccc5oc6c(-c7cccc8c7oc7ccc9ccccc9c78)cccc6c45)n3)ccc2c1 ; c1ccc2cc(-c3nc(-c4ccc5ccccc5c4)nc(-c4cccc5oc6ccc(-c7cccc8c7oc7c9ccccc9ccc87)cc6c45)n3)ccc2c1 ; c1ccc2cc(-c3nc(-c4ccc5ccccc5c4)nc(-c4cc(-c5cccc6c5sc5ccccc56)cc5oc6ccccc6c45)n3)ccc2c1]

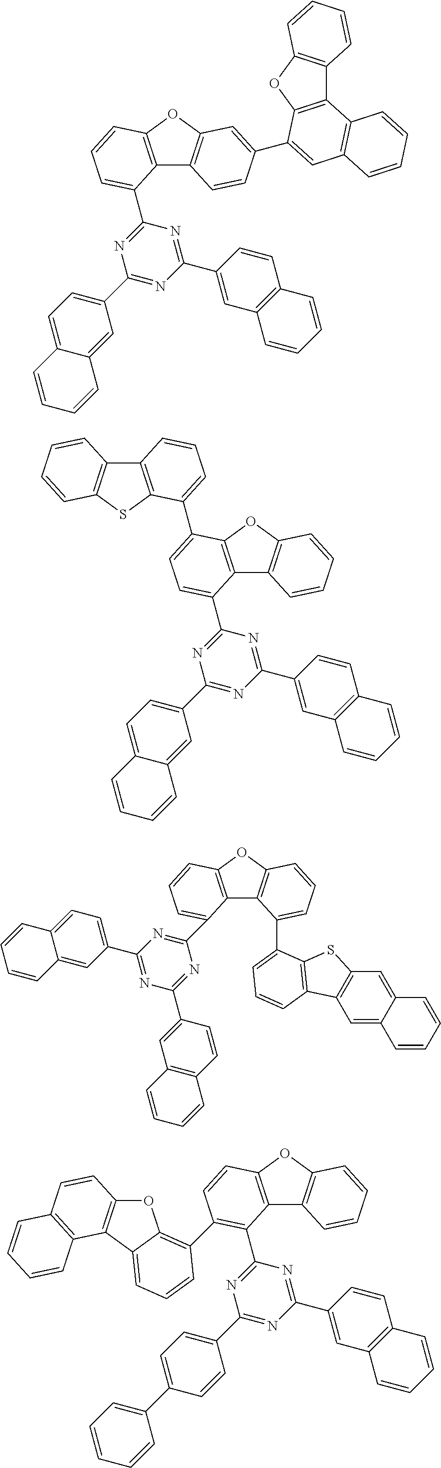

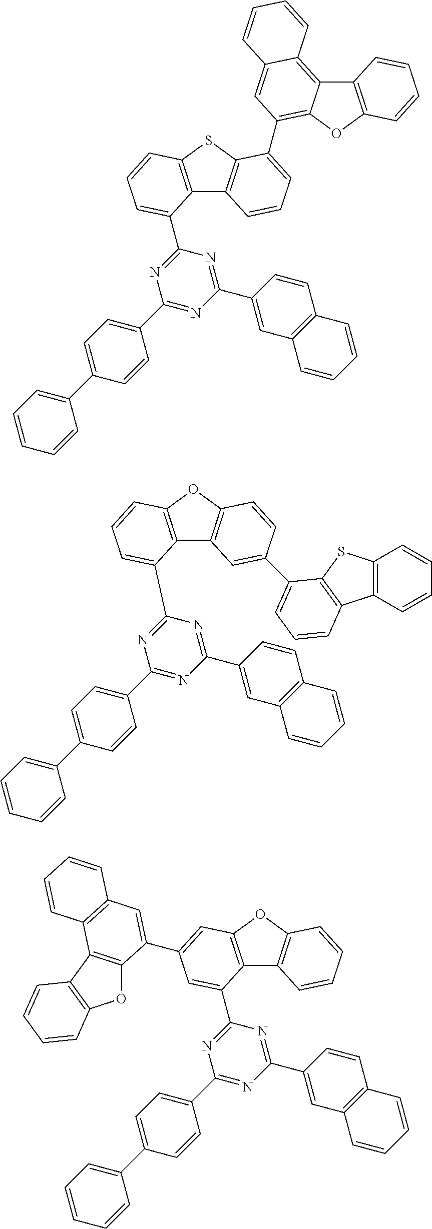

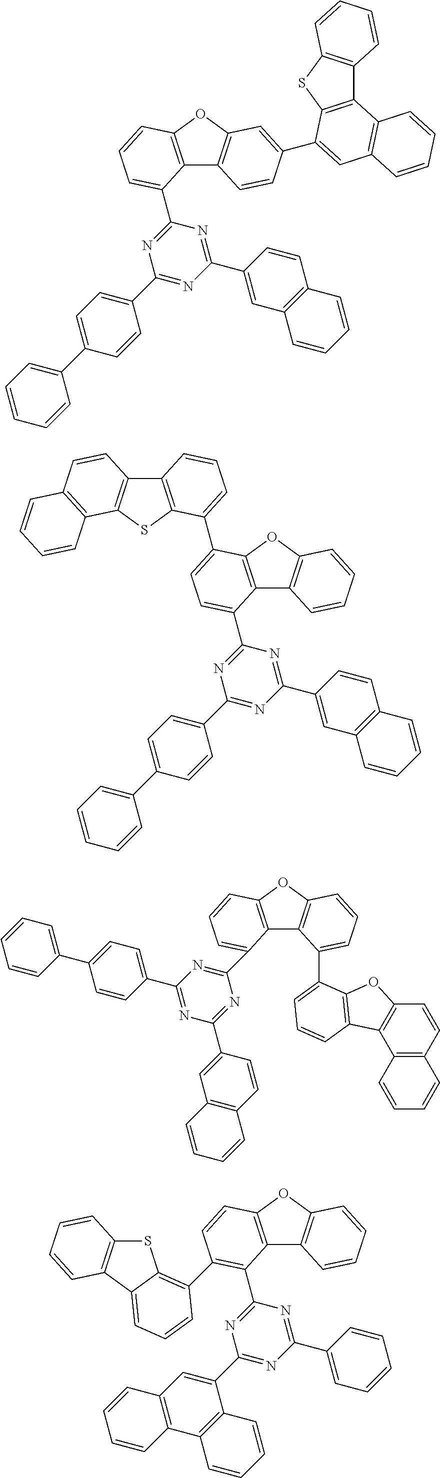

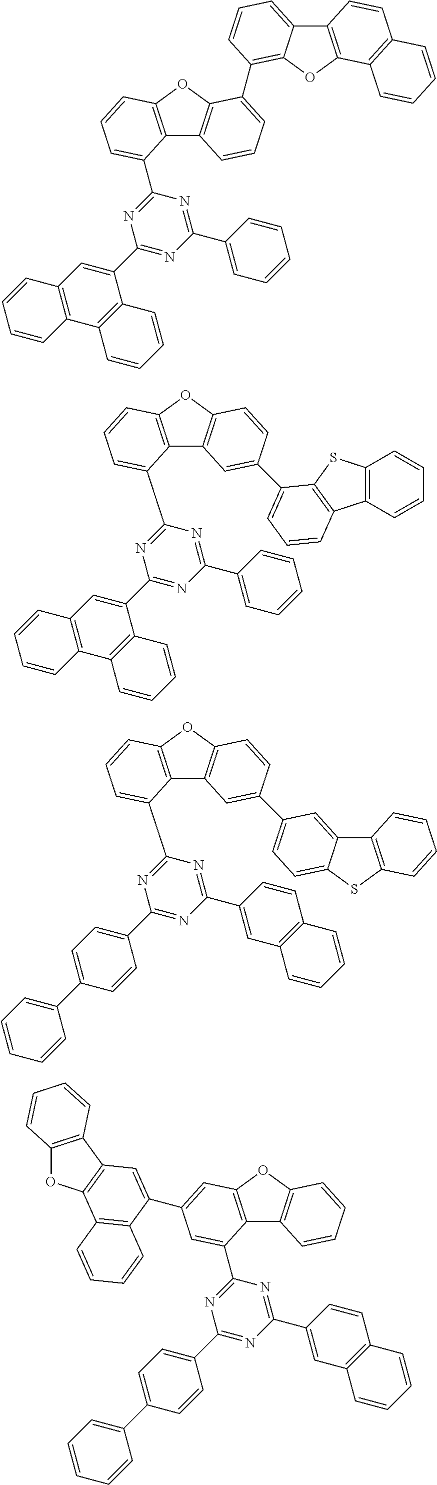

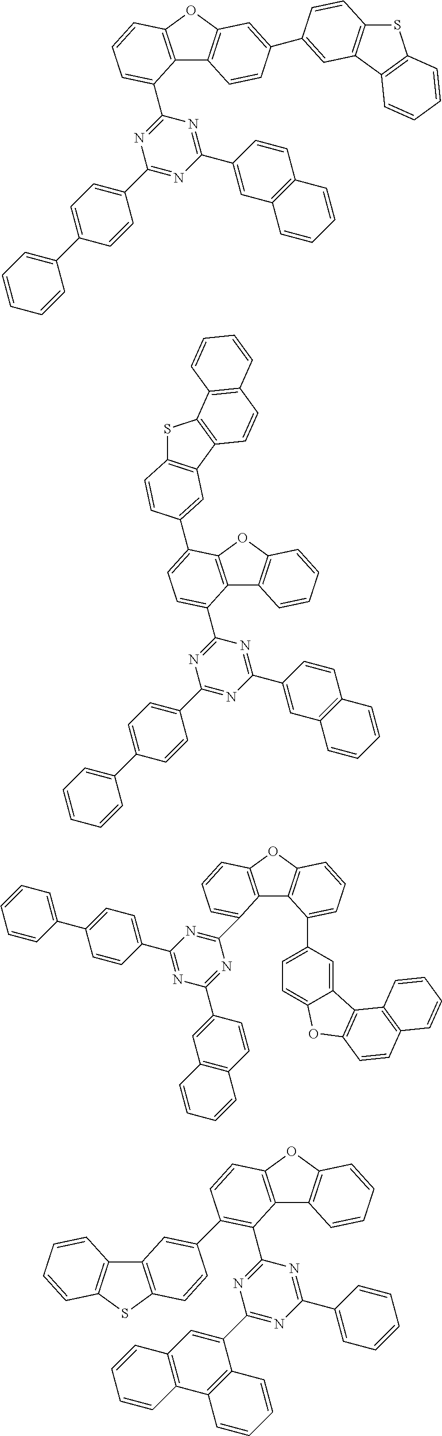 [SMILES: c1ccc(-c2ccc(-c3nc(-c4ccc5ccccc5c4)nc(-c4cccc5oc6cc(-c7ccc8sc9ccccc9c8c7)ccc6c45)n3)cc2)cc1 ; c1ccc(-c2ccc(-c3nc(-c4ccc5ccccc5c4)nc(-c4ccc(-c5ccc6sc7c8ccccc8ccc7c6c5)c5oc6ccccc6c45)n3)cc2)cc1 ; c1ccc(-c2ccc(-c3nc(-c4ccc5ccccc5c4)nc(-c4cccc5oc6cccc(-c7ccc8oc9ccc%10ccccc%10c9c8c7)c6c45)n3)cc2)cc1 ; c1ccc(-c2nc(-c3cc4ccccc4c4ccccc34)nc(-c3c(-c4ccc5sc6ccccc6c5c4)ccc4oc5ccccc5c34)n2)cc1]

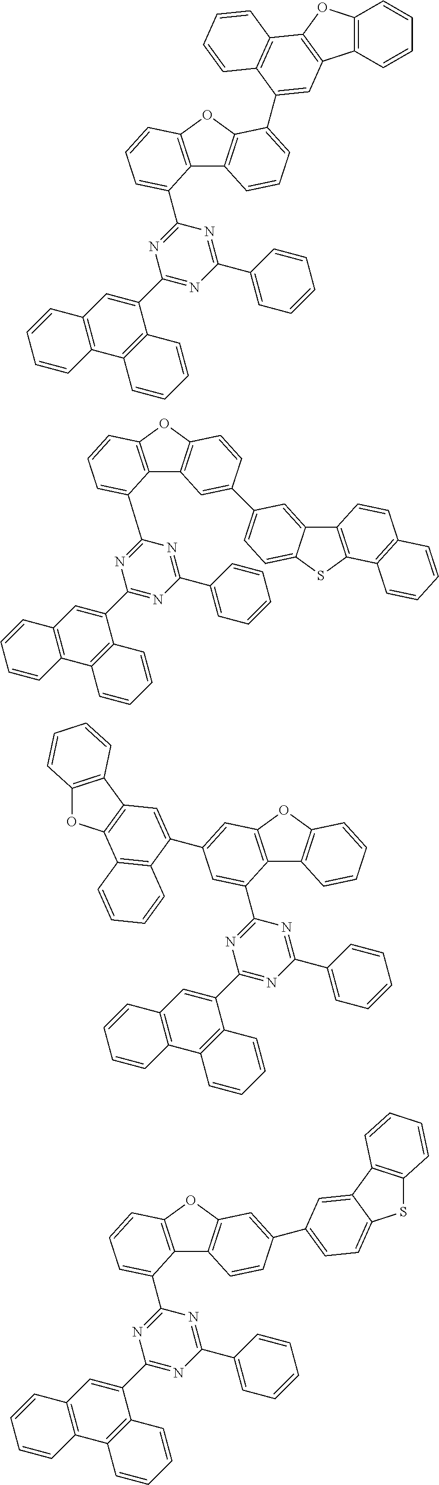

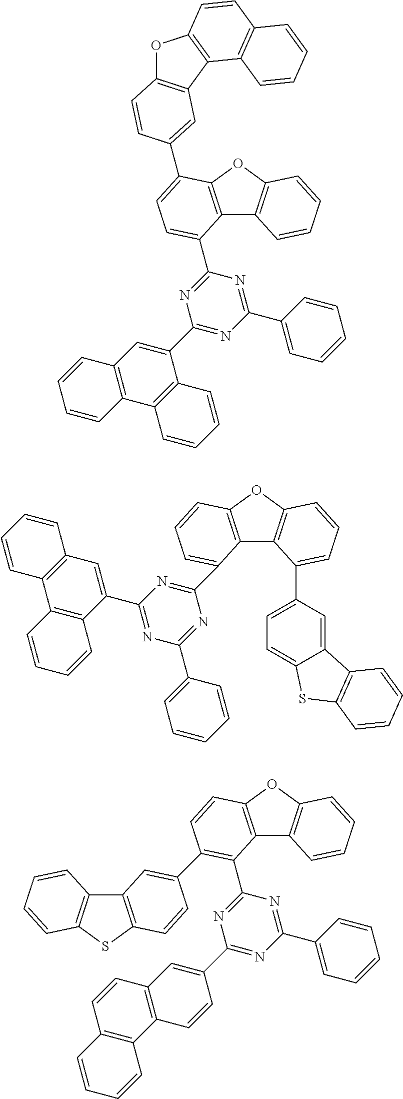 [SMILES: c1ccc(-c2nc(-c3cc4ccccc4c4ccccc34)nc(-c3ccc(-c4ccc5oc6ccc7ccccc7c6c5c4)c4oc5ccccc5c34)n2)cc1 ; c1ccc(-c2nc(-c3cc4ccccc4c4ccccc34)nc(-c3cccc4oc5cccc(-c6ccc7sc8ccccc8c7c6)c5c34)n2)cc1 ; c1ccc(-c2nc(-c3ccc4c(ccc5ccccc54)c3)nc(-c3c(-c4ccc5sc6ccccc6c5c4)ccc4oc5ccccc5c34)n2)cc1]

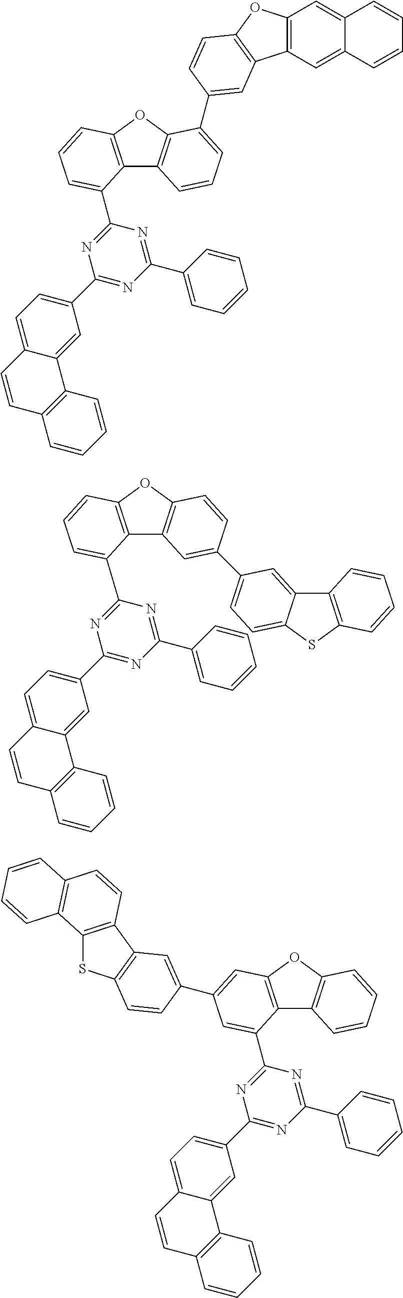

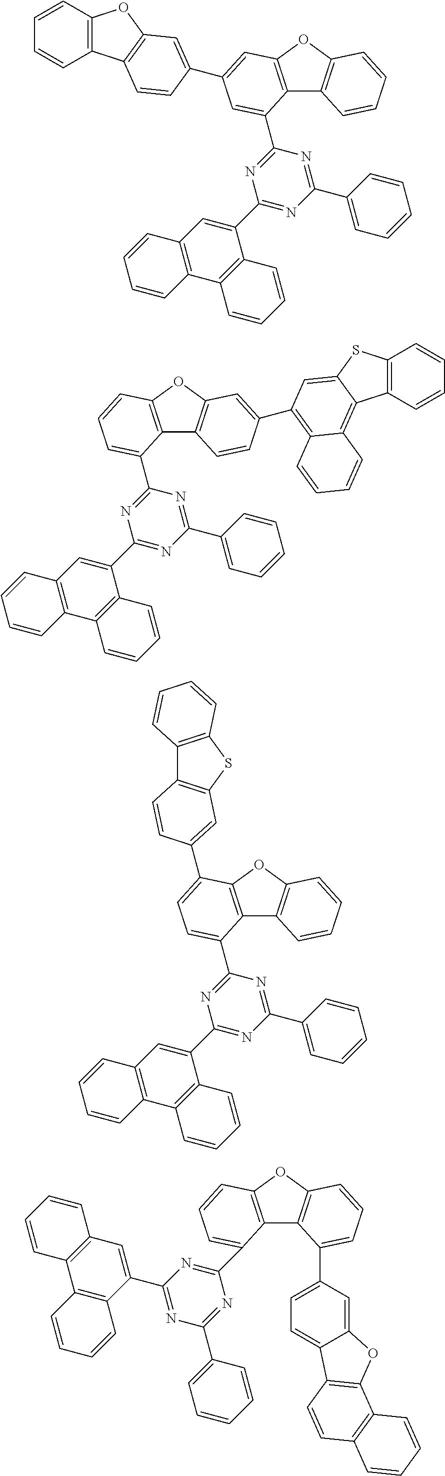

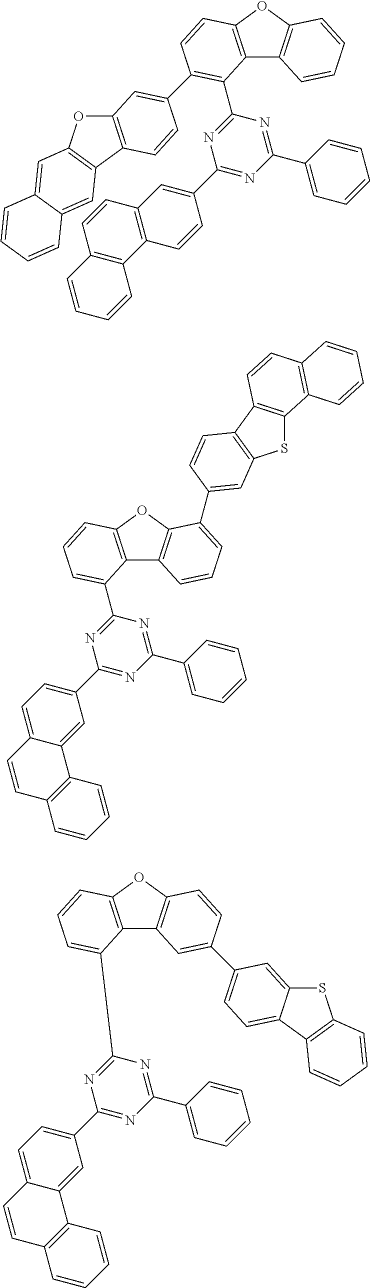 [SMILES: c1ccc(-c2nc(-c3ccc4c(ccc5ccccc54)c3)nc(-c3c(-c4ccc5c(c4)oc4cc6ccccc6cc45)ccc4oc5ccccc5c34)n2)cc1 ; c1ccc(-c2nc(-c3ccc4ccc5ccccc5c4c3)nc(-c3cccc4oc5c(-c6ccc7c(c6)sc6c8ccccc8ccc76)cccc5c34)n2)cc1 ; c1ccc(-c2nc(-c3ccc4ccc5ccccc5c4c3)nc(-c3cccc4oc5ccc(-c6ccc7c(c6)sc6ccccc67)cc5c34)n2)cc1]

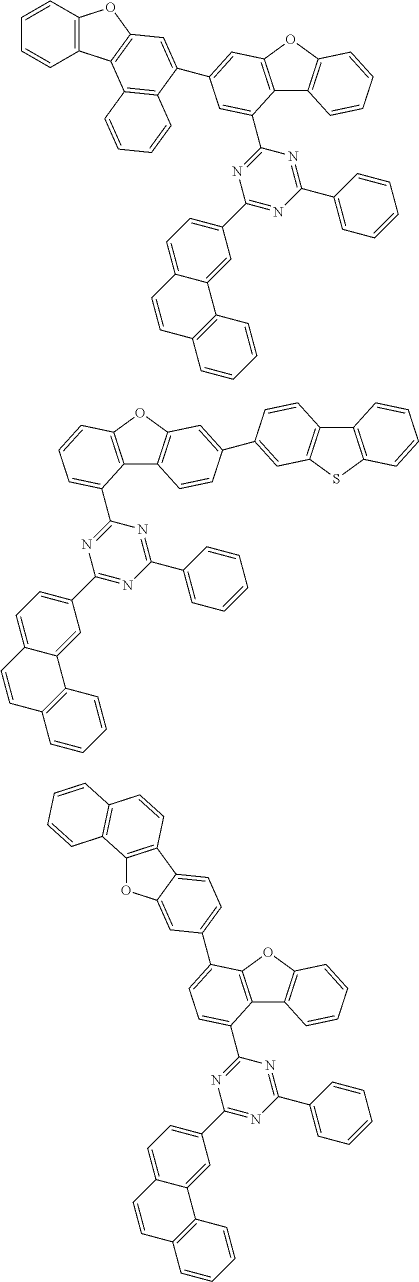

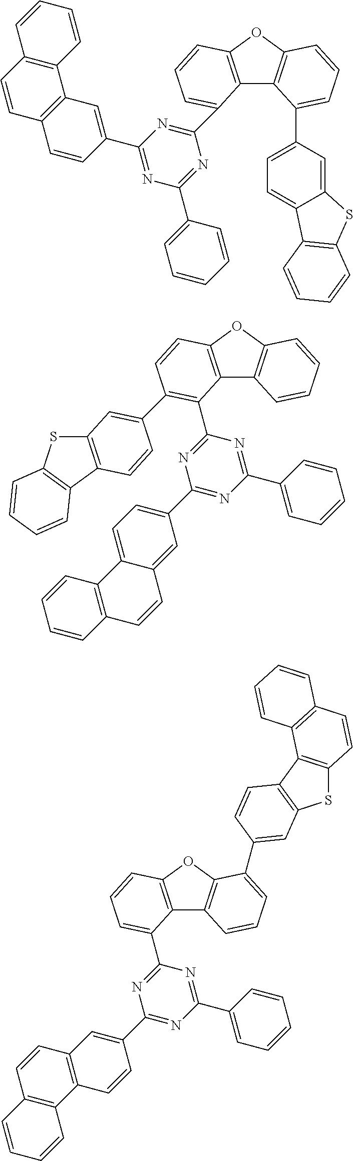

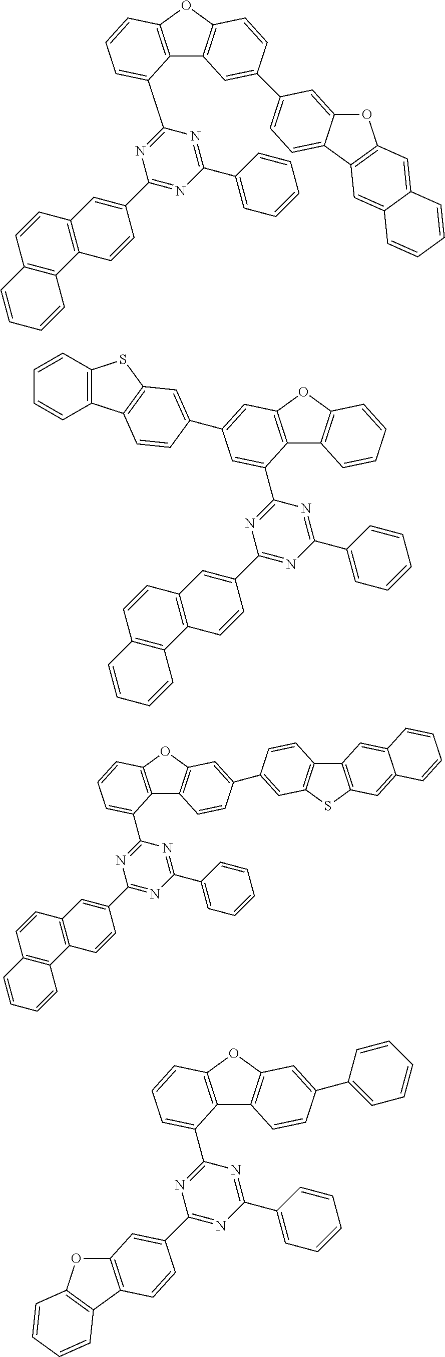

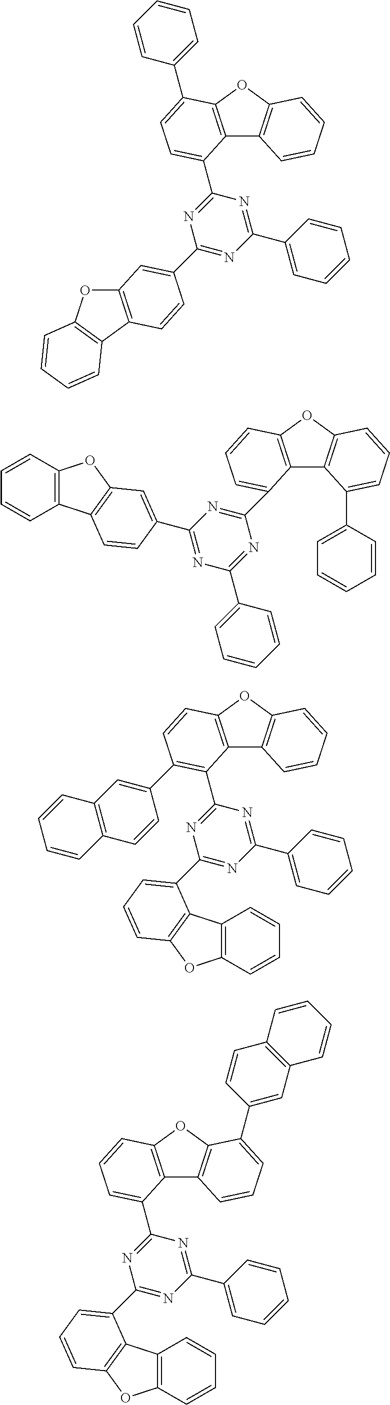

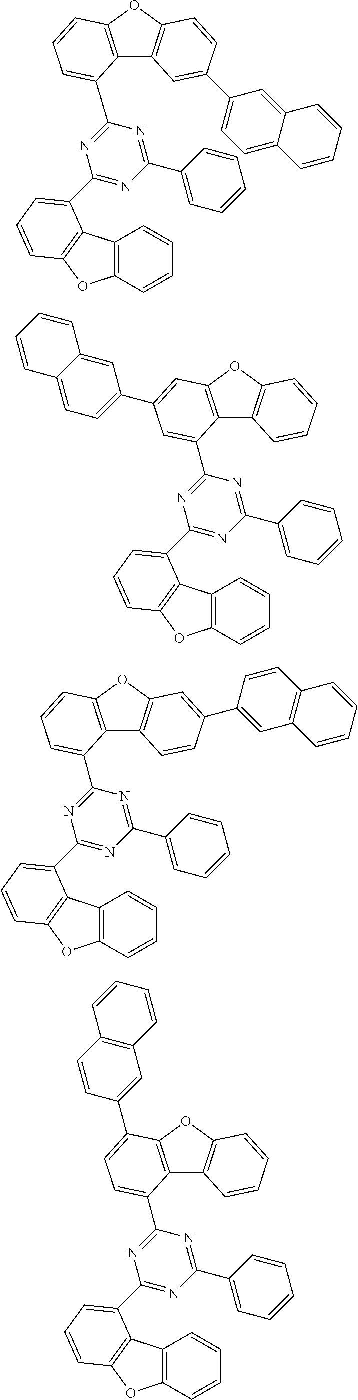

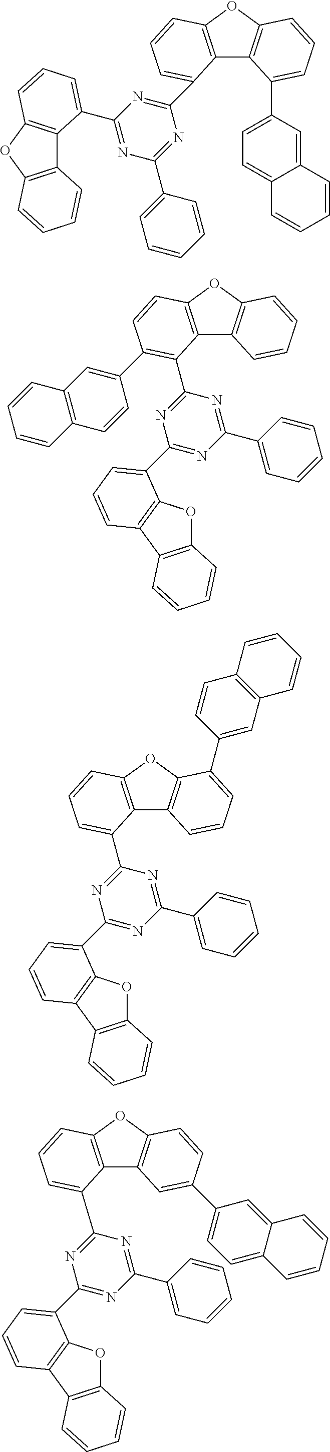

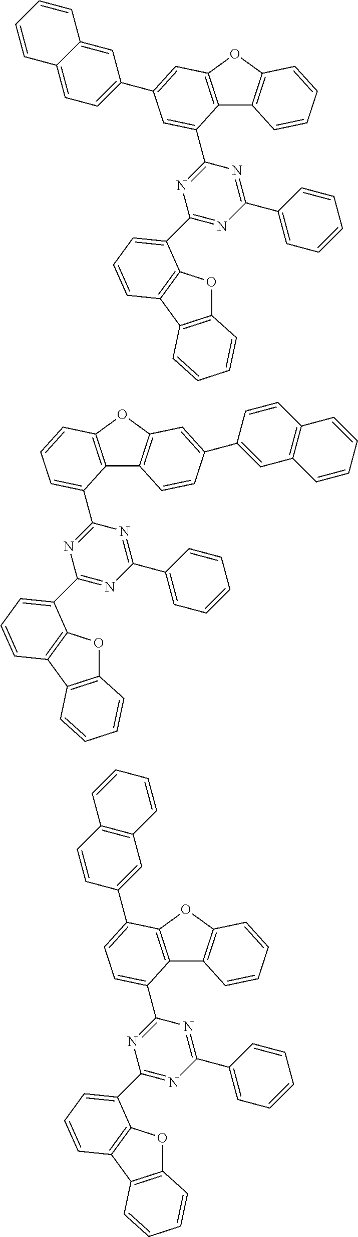

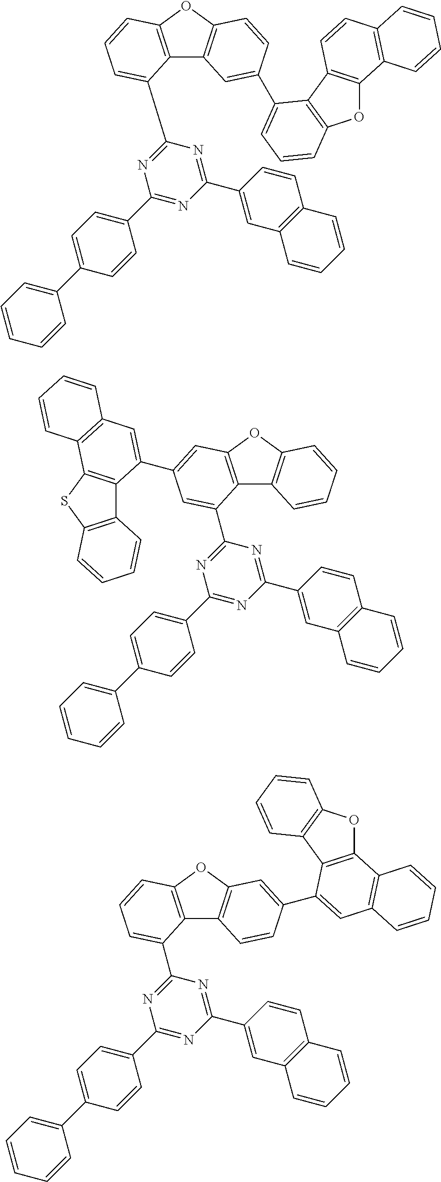

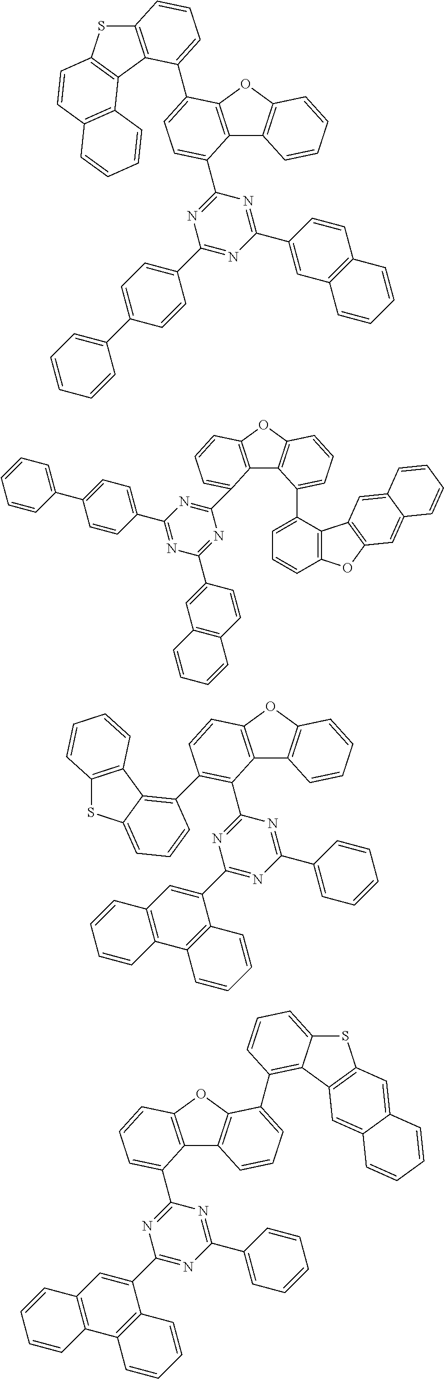

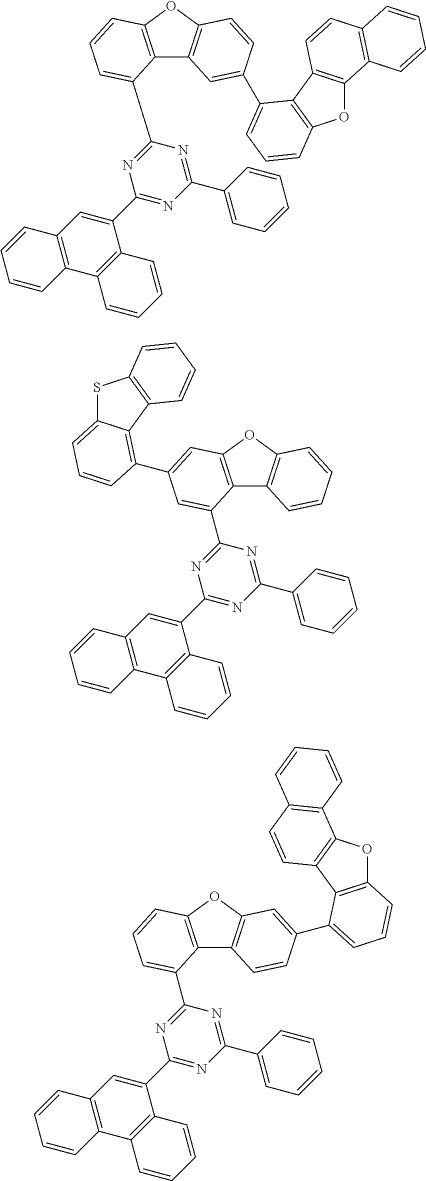

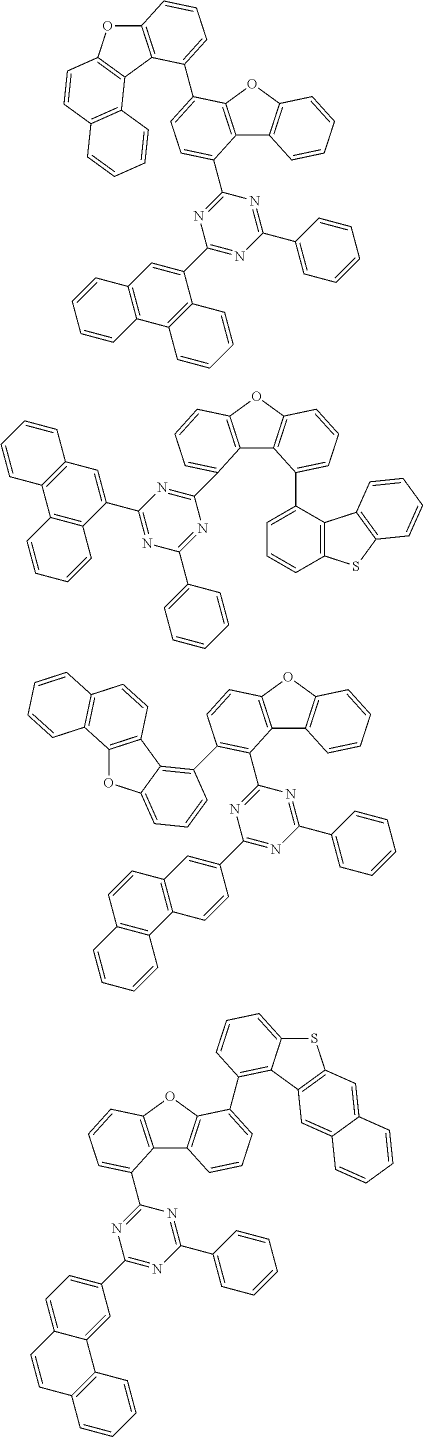

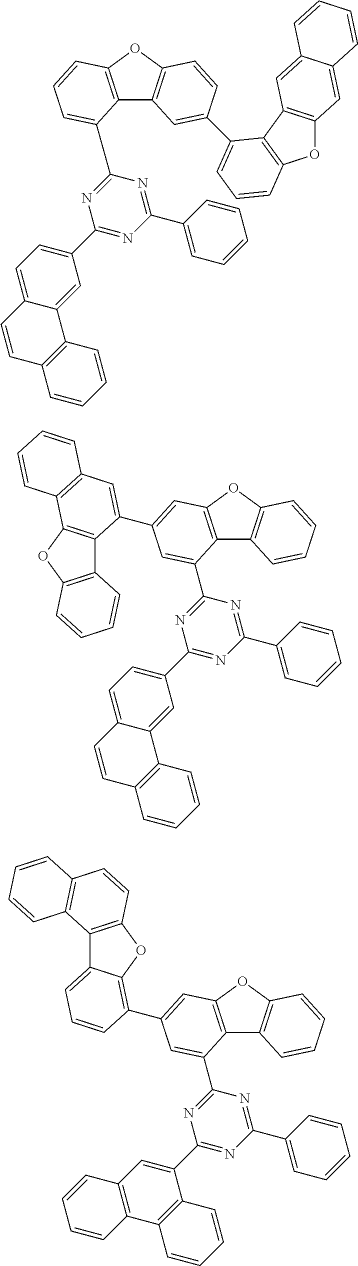

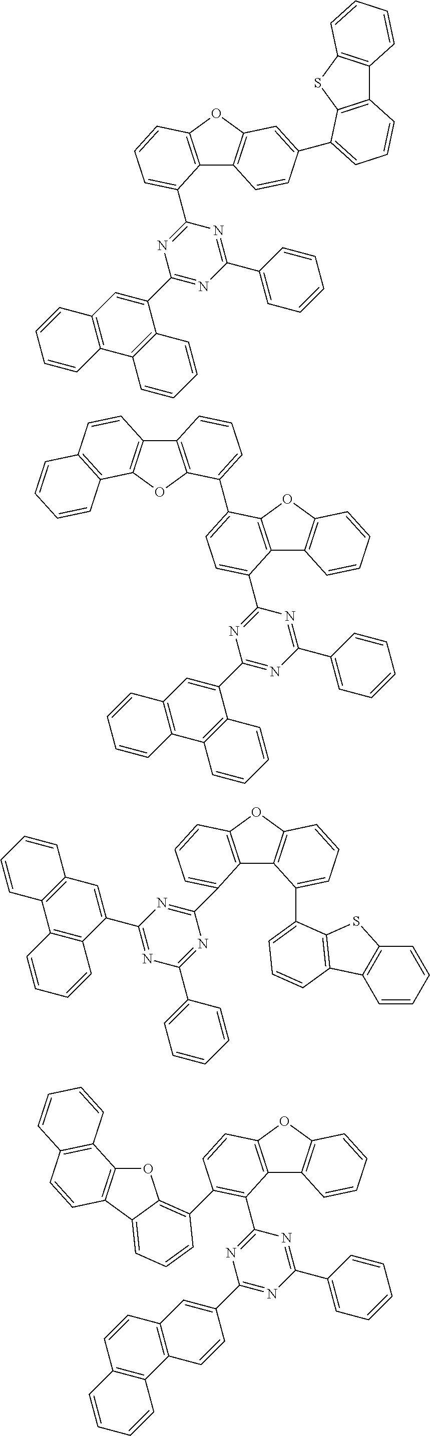

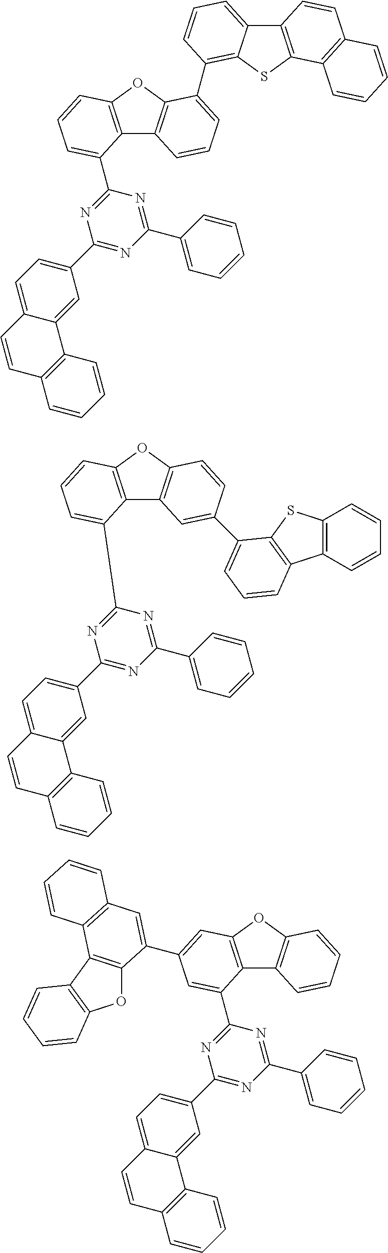

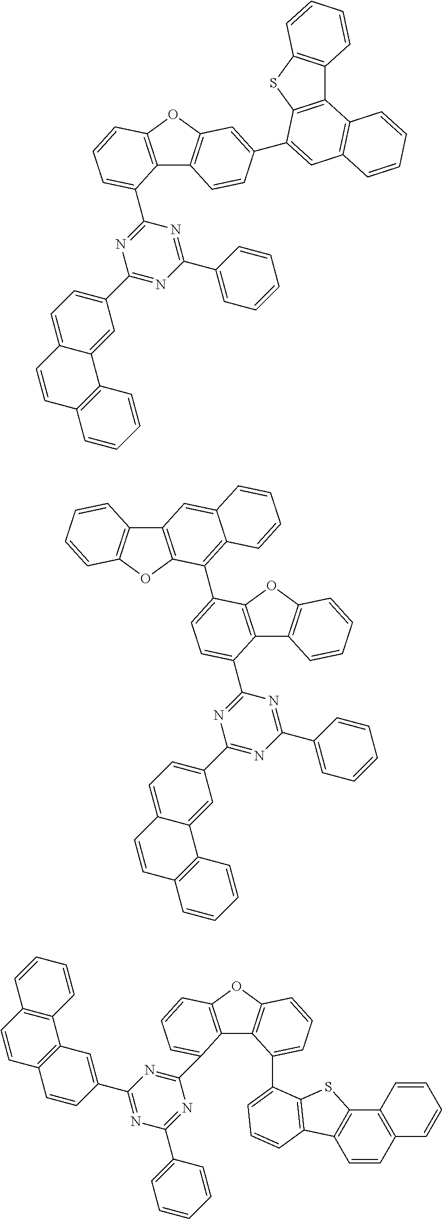 [SMILES: c1ccc(-c2nc(-c3ccc4ccc5ccccc5c4c3)nc(-c3cccc4oc5cc(-c6cc7ccccc7c7c6sc6ccccc67)ccc5c34)n2)cc1 ; c1ccc(-c2nc(-c3ccc4ccc5ccccc5c4c3)nc(-c3ccc(-c4c5ccccc5cc5c4oc4ccccc45)c4oc5ccccc5c34)n2)cc1 ; c1ccc(-c2nc(-c3ccc4ccc5ccccc5c4c3)nc(-c3cccc4oc5cccc(-c6cccc7c6sc6c8ccccc8ccc76)c5c34)n2)cc1]

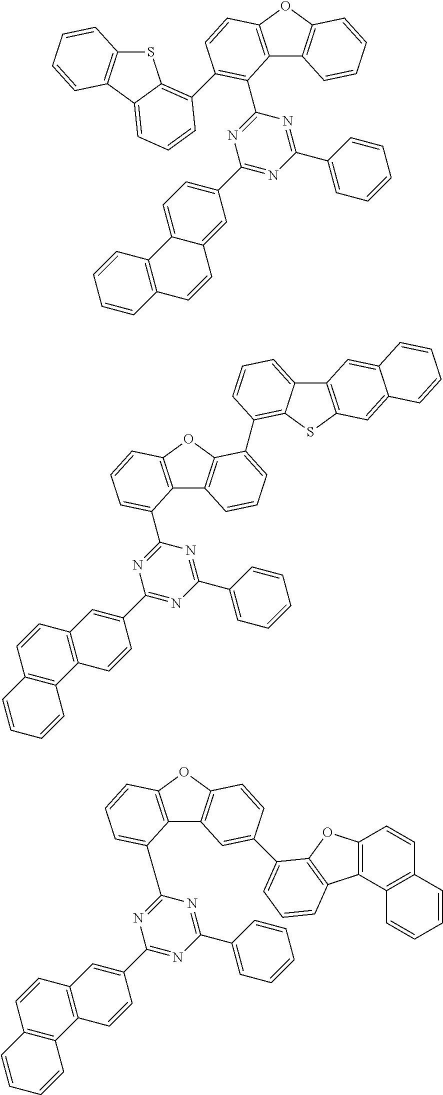 [SMILES: c1ccc(-c2nc(-c3ccc4c(ccc5ccccc54)c3)nc(-c3c(-c4cccc5c4sc4ccccc45)ccc4oc5ccccc5c34)n2)cc1 ; c1ccc(-c2nc(-c3ccc4c(ccc5ccccc54)c3)nc(-c3cccc4oc5c(-c6cccc7c6sc6cc8ccccc8cc67)cccc5c34)n2)cc1 ; c1ccc(-c2nc(-c3ccc4c(ccc5ccccc54)c3)nc(-c3cccc4oc5ccc(-c6cccc7c6oc6ccc8ccccc8c67)cc5c34)n2)cc1]

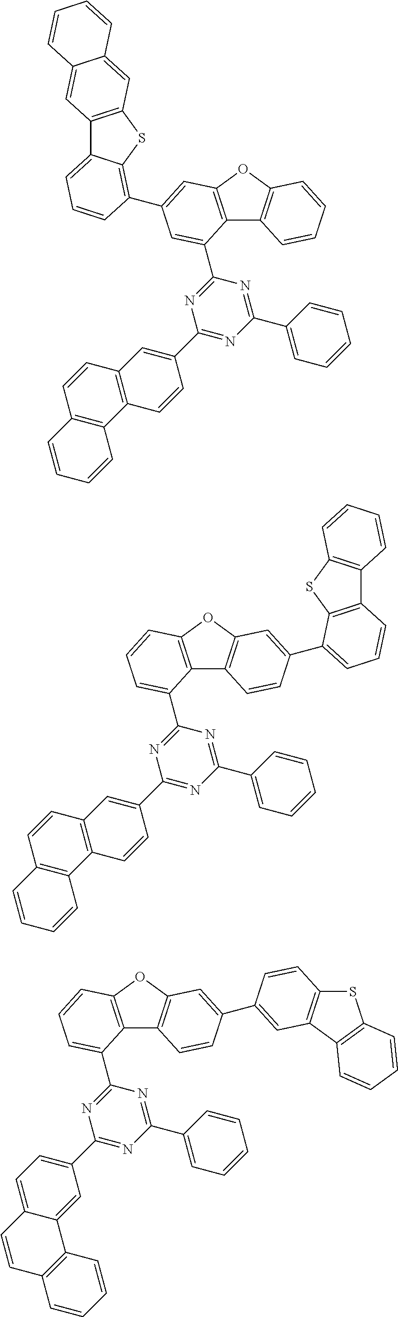 [SMILES: c1ccc(-c2nc(-c3ccc4c(ccc5ccccc54)c3)nc(-c3cc(-c4cccc5c4sc4cc6ccccc6cc45)cc4oc5ccccc5c34)n2)cc1 ; c1ccc(-c2nc(-c3ccc4c(ccc5ccccc54)c3)nc(-c3cccc4oc5cc(-c6cccc7c6sc6ccccc67)ccc5c34)n2)cc1 ; c1ccc(-c2nc(-c3ccc4ccc5ccccc5c4c3)nc(-c3cccc4oc5cc(-c6ccc7sc8ccccc8c7c6)ccc5c34)n2)cc1]

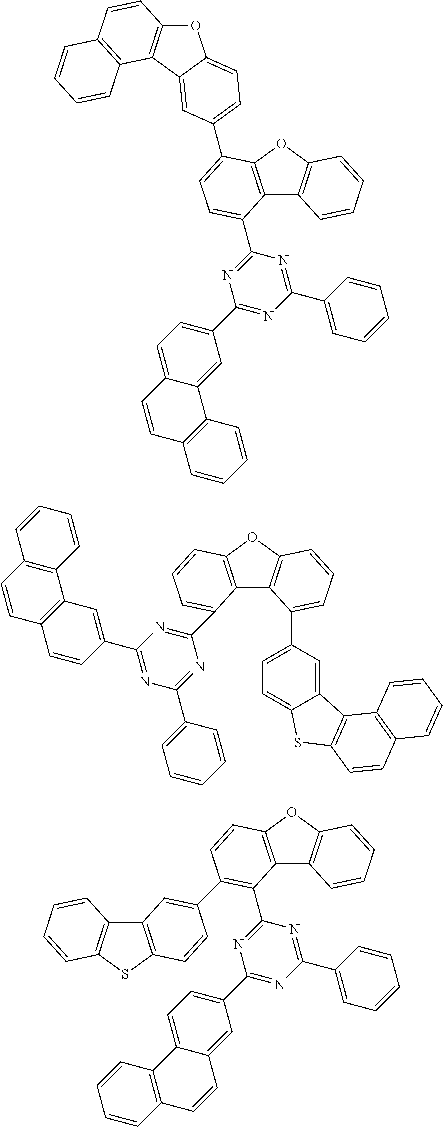 [SMILES: c1ccc(-c2nc(-c3ccc4ccc5ccccc5c4c3)nc(-c3ccc(-c4ccc5oc6ccc7ccccc7c6c5c4)c4oc5ccccc5c34)n2)cc1 ; c1ccc(-c2nc(-c3ccc4ccc5ccccc5c4c3)nc(-c3cccc4oc5cccc(-c6ccc7sc8ccc9ccccc9c8c7c6)c5c34)n2)cc1 ; c1ccc(-c2nc(-c3ccc4c(ccc5ccccc54)c3)nc(-c3c(-c4ccc5sc6ccccc6c5c4)ccc4oc5ccccc5c34)n2)cc1]

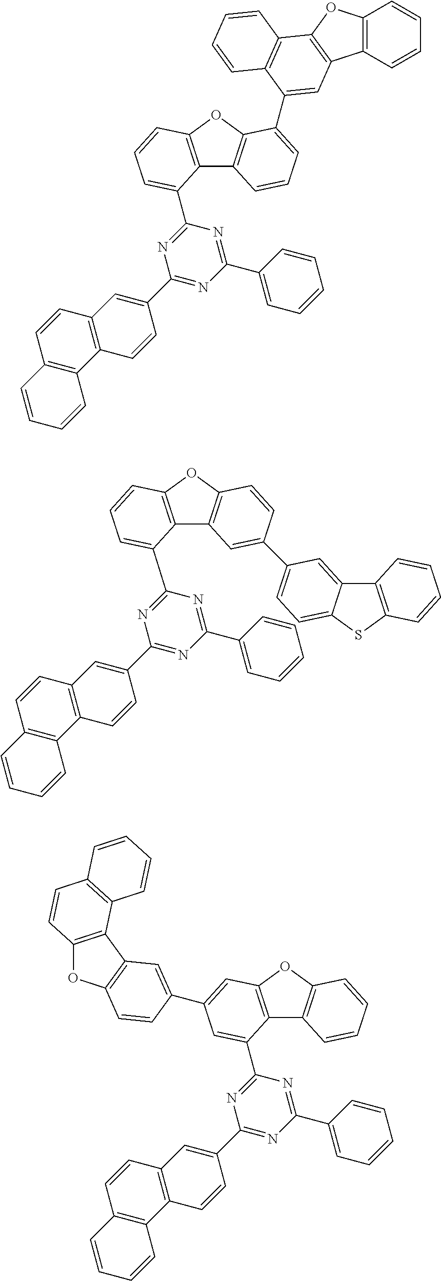 [SMILES: c1ccc(-c2nc(-c3ccc4c(ccc5ccccc54)c3)nc(-c3cccc4oc5c(-c6cc7c8ccccc8oc7c7ccccc67)cccc5c34)n2)cc1 ; c1ccc(-c2nc(-c3ccc4c(ccc5ccccc54)c3)nc(-c3cccc4oc5ccc(-c6ccc7sc8ccccc8c7c6)cc5c34)n2)cc1 ; c1ccc(-c2nc(-c3ccc4c(ccc5ccccc54)c3)nc(-c3cc(-c4ccc5oc6ccc7ccccc7c6c5c4)cc4oc5ccccc5c34)n2)cc1]

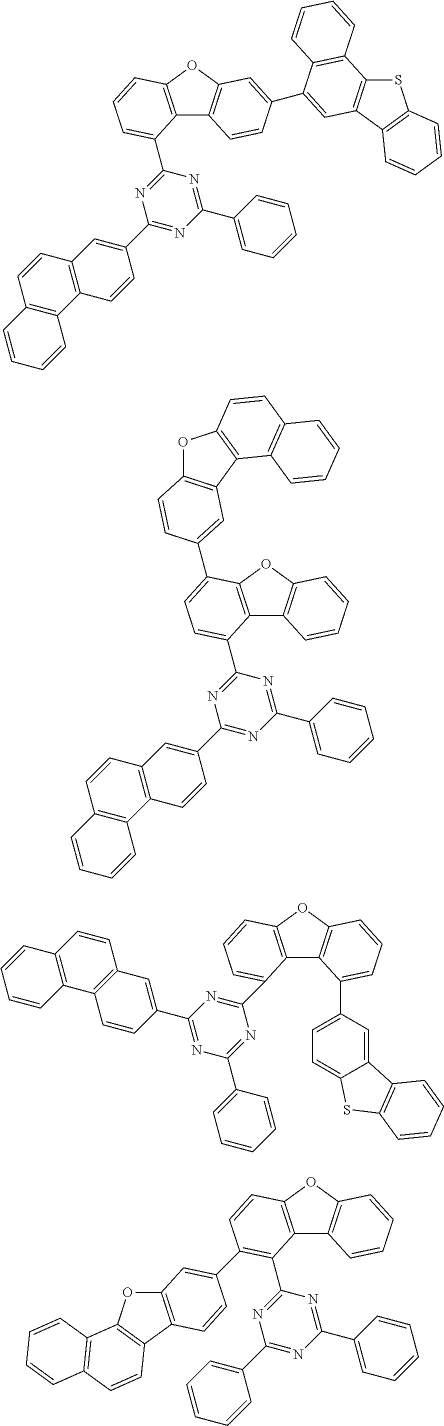

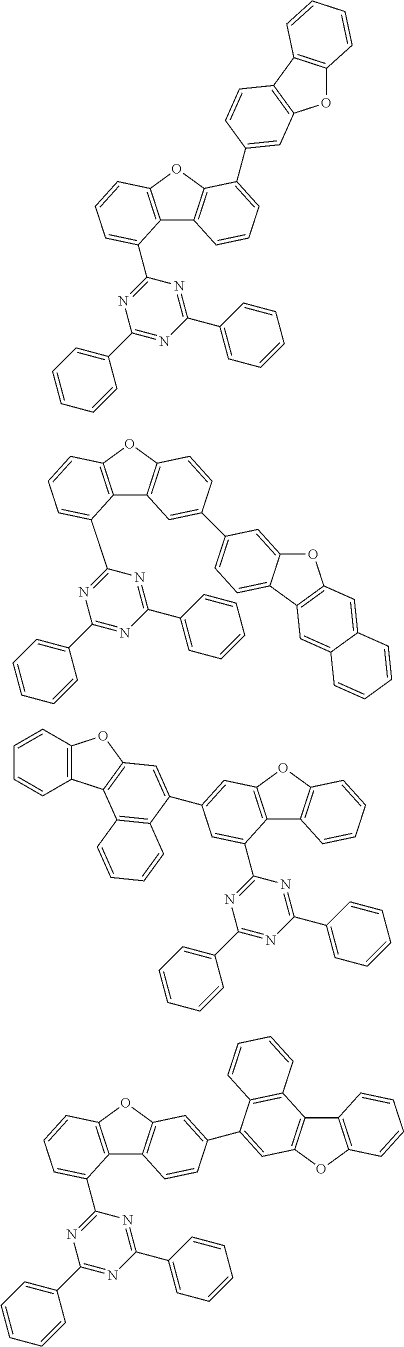

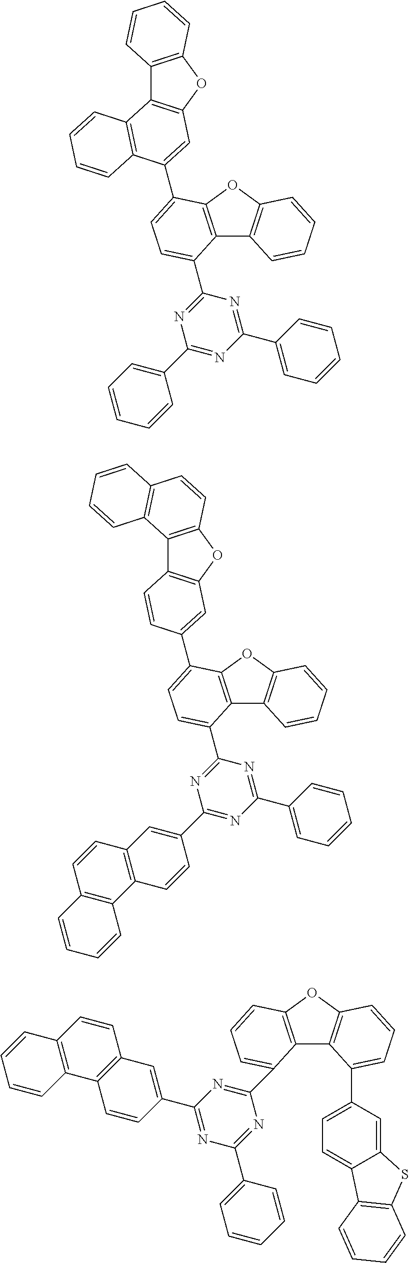

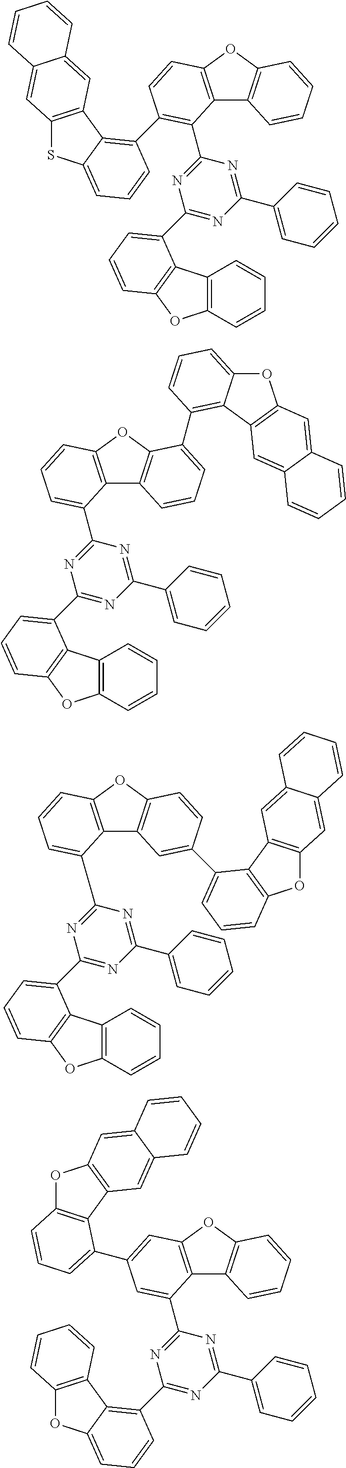

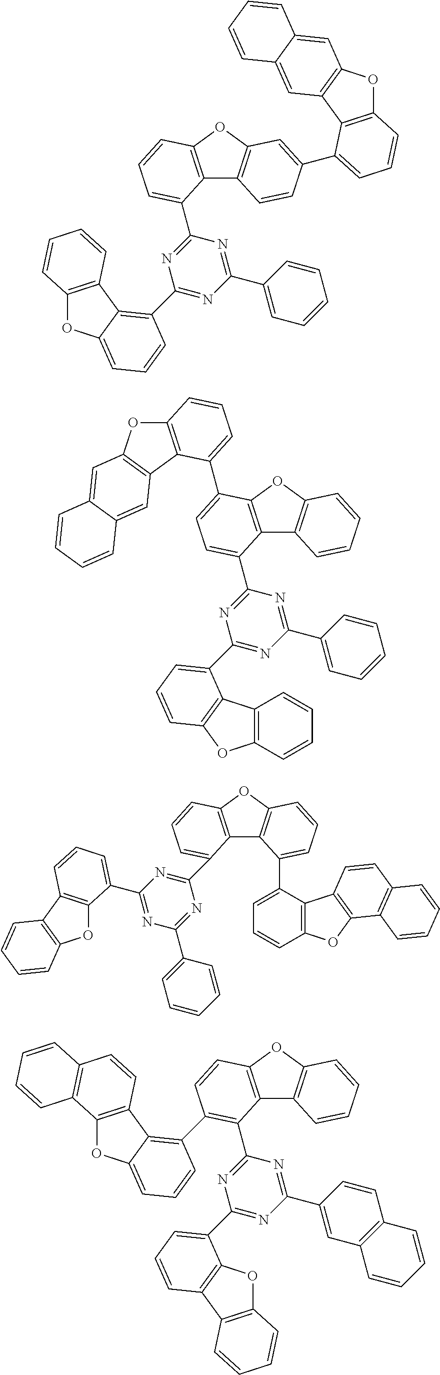

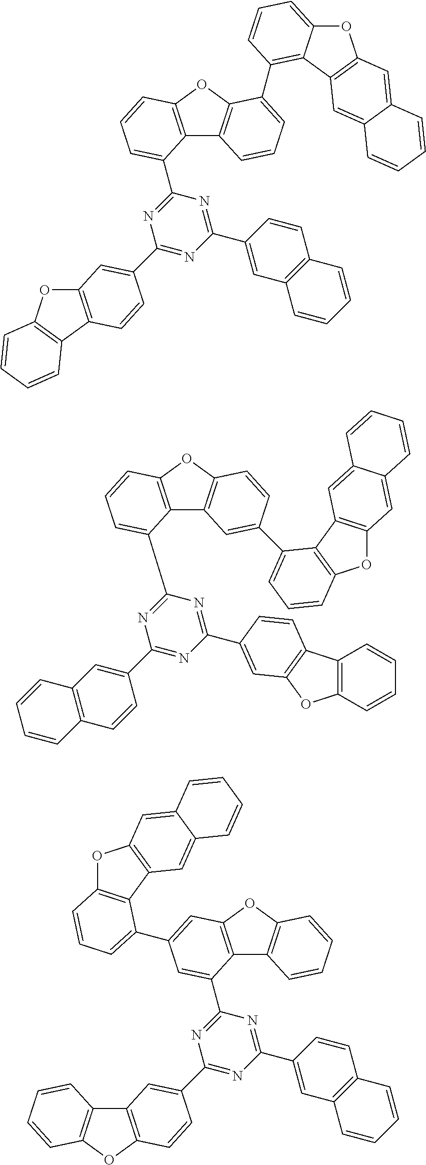 [SMILES: c1ccc2cc(-c3nc(-c4ccc5c(c4)oc4ccccc45)nc(-c4cccc5oc6c(-c7cccc8oc9cc%10ccccc%10cc9c78)cccc6c45)n3)ccc2c1 ; c1ccc2cc(-c3nc(-c4ccc5c(c4)oc4ccccc45)nc(-c4cccc5oc6ccc(-c7cccc8oc9cc%10ccccc%10cc9c78)cc6c45)n3)ccc2c1 ; c1ccc2cc(-c3nc(-c4ccc5oc6ccccc6c5c4)nc(-c4cc(-c5cccc6oc7cc8ccccc8cc7c56)cc5oc6ccccc6c45)n3)ccc2c1]

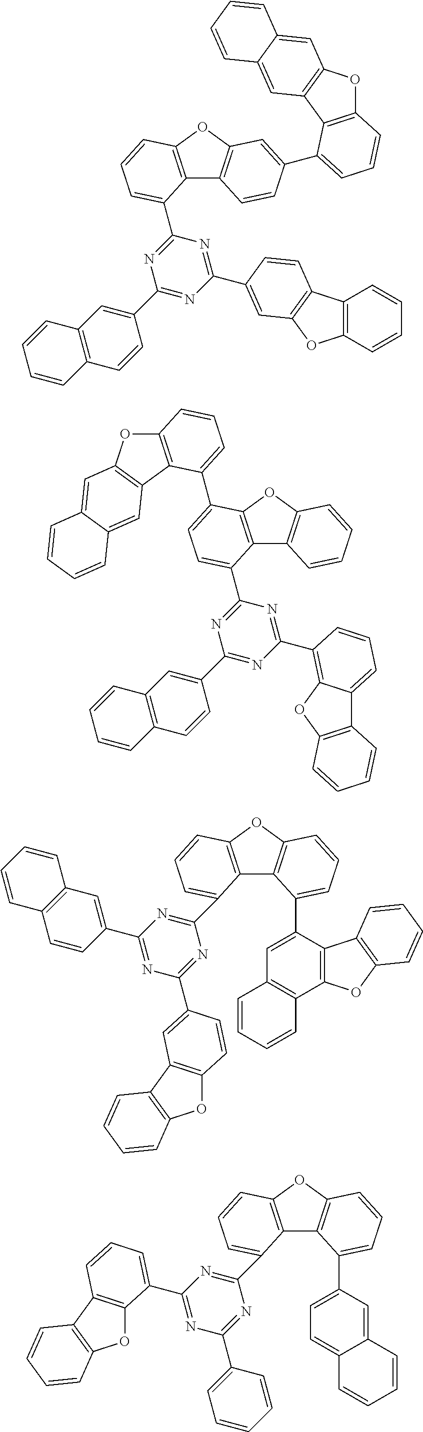 [SMILES: c1ccc2cc(-c3nc(-c4ccc5c(c4)oc4ccccc45)nc(-c4cccc5oc6cc(-c7cccc8oc9cc%10ccccc%10cc9c78)ccc6c45)n3)ccc2c1 ; c1ccc2cc(-c3nc(-c4cccc5c4oc4ccccc45)nc(-c4ccc(-c5cccc6oc7cc8ccccc8cc7c56)c5oc6ccccc6c45)n3)ccc2c1 ; c1ccc2cc(-c3nc(-c4ccc5oc6ccccc6c5c4)nc(-c4cccc5oc6cccc(-c7cc8ccccc8c8oc9ccccc9c78)c6c45)n3)ccc2c1 ; c1ccc(-c2nc(-c3cccc4c3oc3ccccc34)nc(-c3cccc4oc5cccc(-c6ccc7ccccc7c6)c5c34)n2)cc1]

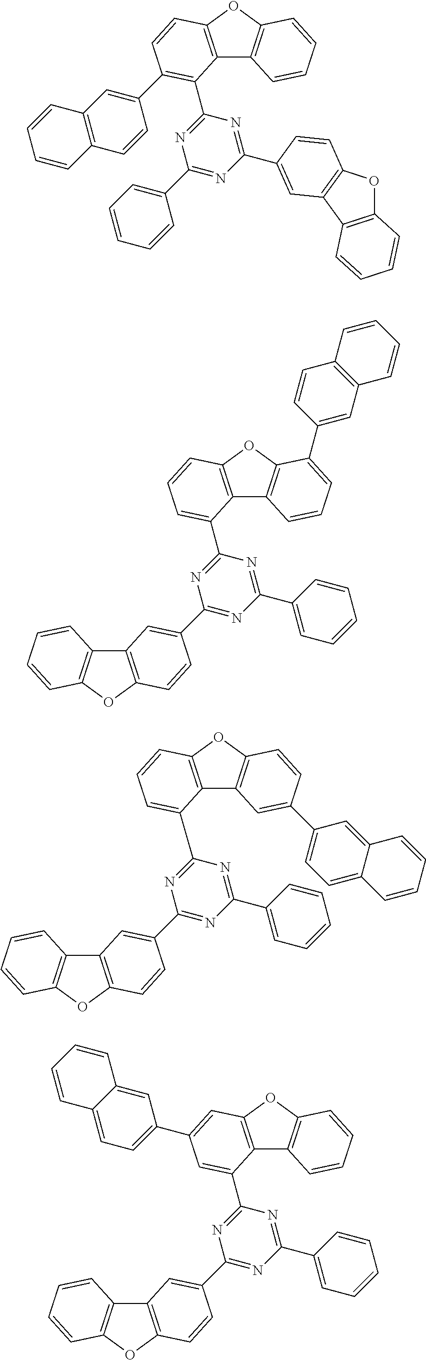

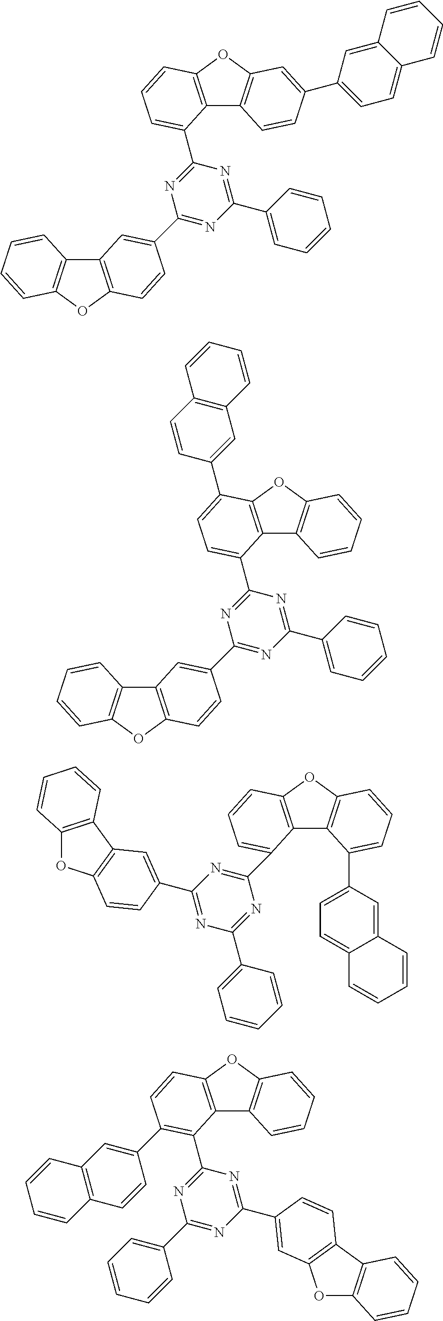

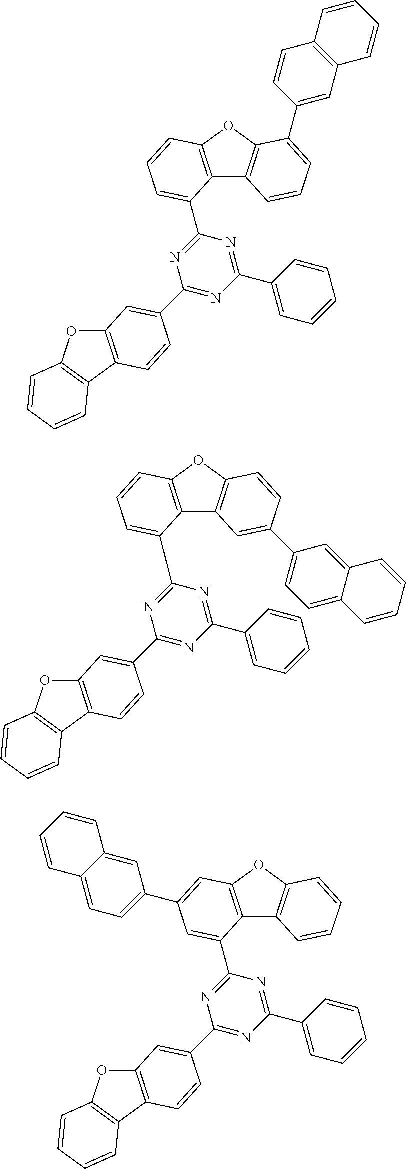 [SMILES: c1ccc(-c2nc(-c3ccc4c(c3)oc3ccccc34)nc(-c3cccc4oc5c(-c6ccc7ccccc7c6)cccc5c34)n2)cc1 ; c1ccc(-c2nc(-c3ccc4c(c3)oc3ccccc34)nc(-c3cccc4oc5ccc(-c6ccc7ccccc7c6)cc5c34)n2)cc1 ; c1ccc(-c2nc(-c3ccc4c(c3)oc3ccccc34)nc(-c3cc(-c4ccc5ccccc5c4)cc4oc5ccccc5c34)n2)cc1]

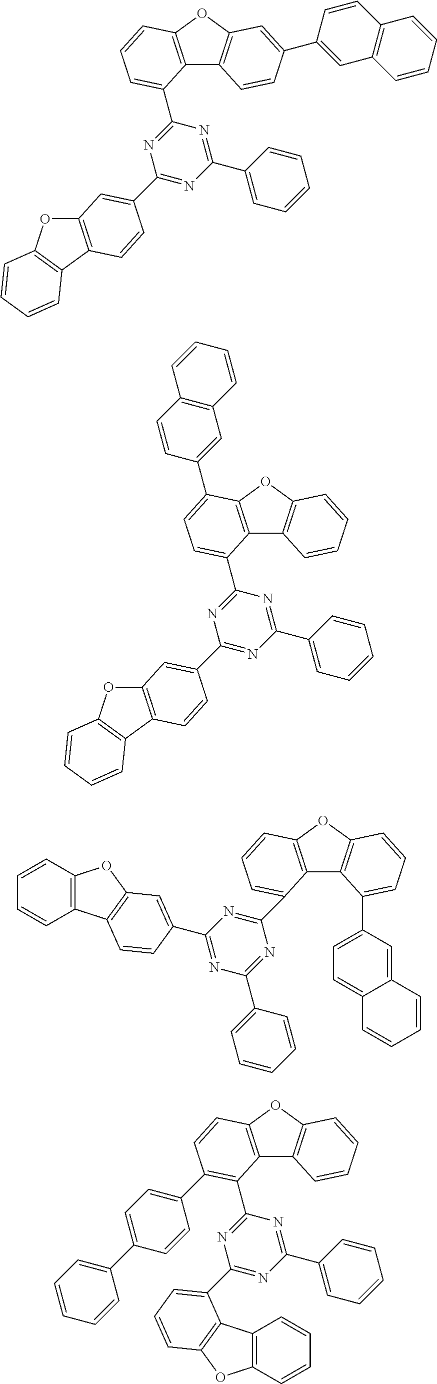

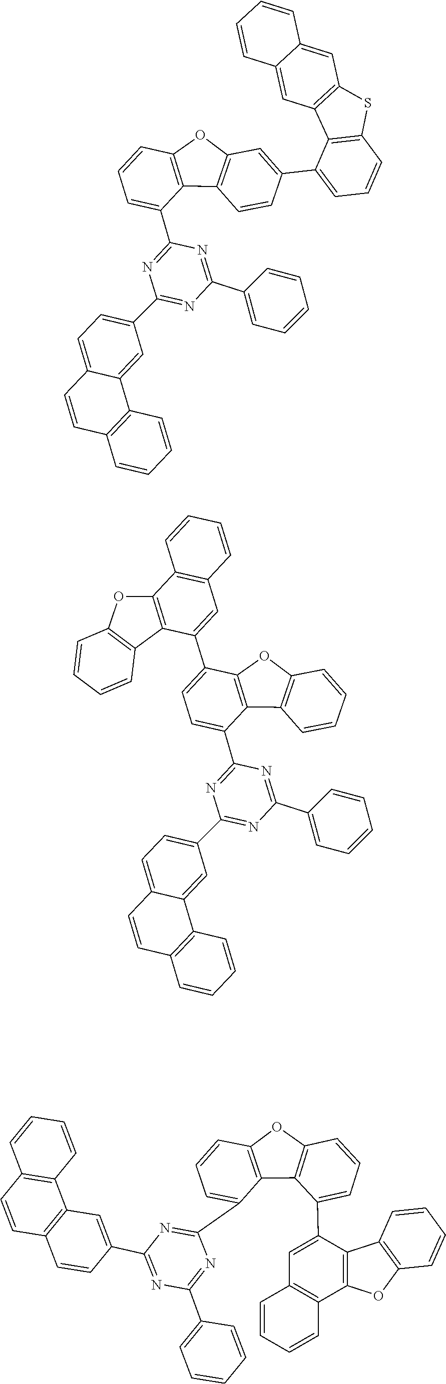 [SMILES: c1ccc(-c2nc(-c3ccc4ccc5ccccc5c4c3)nc(-c3cccc4oc5cc(-c6cccc7sc8cc9ccccc9cc8c67)ccc5c34)n2)cc1 ; c1ccc(-c2nc(-c3ccc4ccc5ccccc5c4c3)nc(-c3ccc(-c4cc5ccccc5c5oc6ccccc6c45)c4oc5ccccc5c34)n2)cc1 ; c1ccc(-c2nc(-c3ccc4ccc5ccccc5c4c3)nc(-c3cccc4oc5cccc(-c6cc7ccccc7c7oc8ccccc8c67)c5c34)n2)cc1]

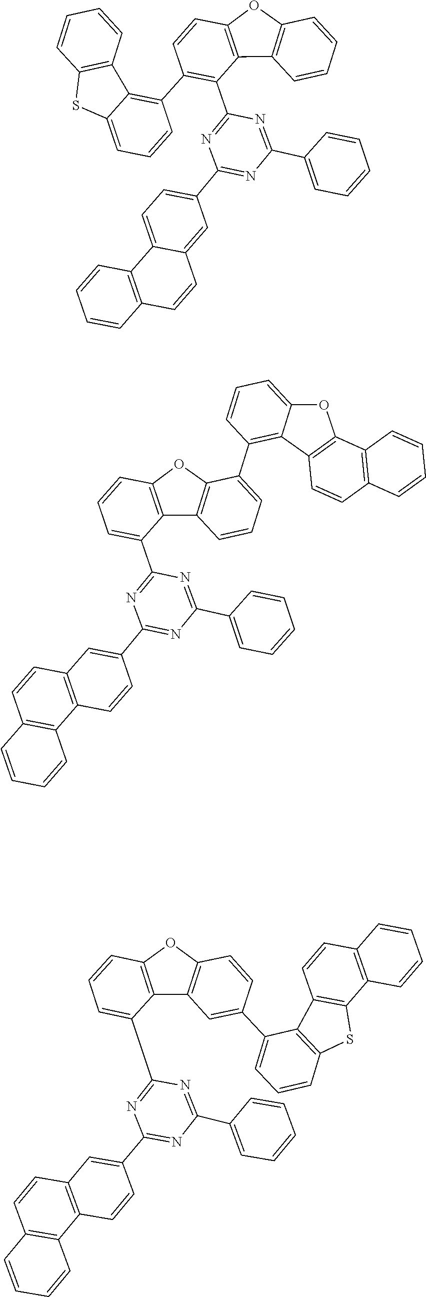 [SMILES: c1ccc(-c2nc(-c3ccc4c(ccc5ccccc54)c3)nc(-c3c(-c4cccc5sc6ccccc6c45)ccc4oc5ccccc5c34)n2)cc1 ; c1ccc(-c2nc(-c3ccc4c(ccc5ccccc54)c3)nc(-c3cccc4oc5c(-c6cccc7oc8c9ccccc9ccc8c67)cccc5c34)n2)cc1 ; c1ccc(-c2nc(-c3ccc4c(ccc5ccccc54)c3)nc(-c3cccc4oc5ccc(-c6cccc7sc8c9ccccc9ccc8c67)cc5c34)n2)cc1]

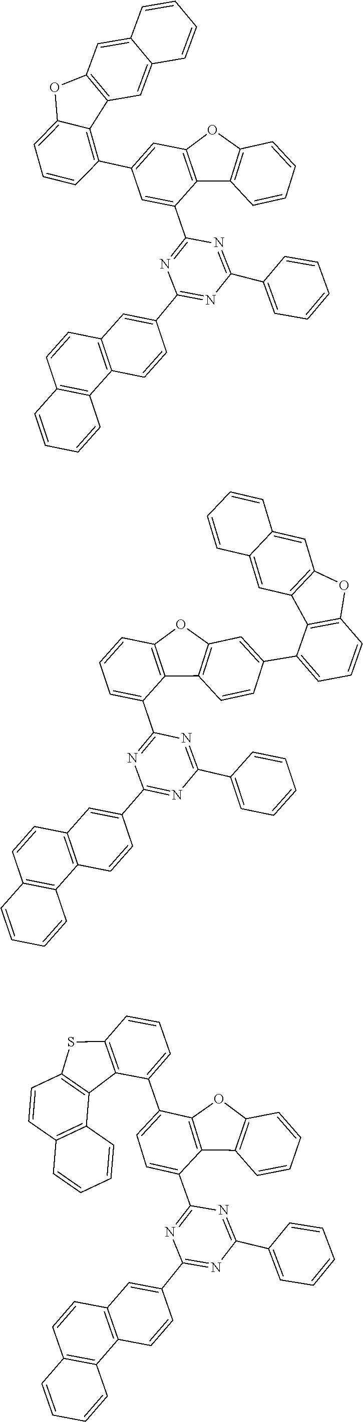 [SMILES: c1ccc(-c2nc(-c3ccc4c(ccc5ccccc54)c3)nc(-c3cc(-c4cccc5oc6cc7ccccc7cc6c45)cc4oc5ccccc5c34)n2)cc1 ; c1ccc(-c2nc(-c3ccc4c(ccc5ccccc54)c3)nc(-c3cccc4oc5cc(-c6cccc7oc8cc9ccccc9cc8c67)ccc5c34)n2)cc1 ; c1ccc(-c2nc(-c3ccc4c(ccc5ccccc54)c3)nc(-c3ccc(-c4cccc5sc6ccc7ccccc7c6c45)c4oc5ccccc5c34)n2)cc1]

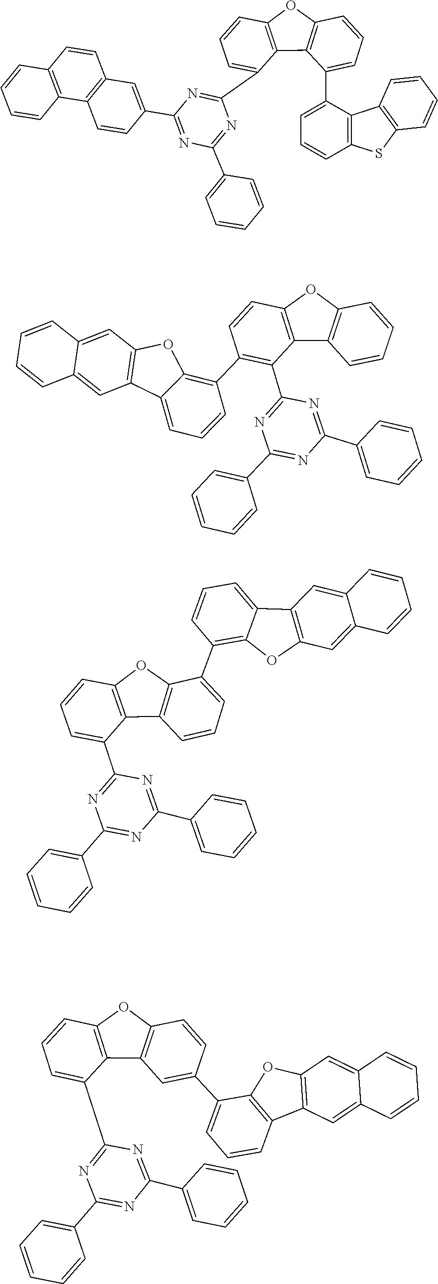 [SMILES: c1ccc(-c2nc(-c3ccc4c(ccc5ccccc54)c3)nc(-c3cccc4oc5cccc(-c6cccc7sc8ccccc8c67)c5c34)n2)cc1 ; c1ccc(-c2nc(-c3ccccc3)nc(-c3c(-c4cccc5c4oc4cc6ccccc6cc45)ccc4oc5ccccc5c34)n2)cc1 ; c1ccc(-c2nc(-c3ccccc3)nc(-c3cccc4oc5c(-c6cccc7c6oc6cc8ccccc8cc67)cccc5c34)n2)cc1 ; c1ccc(-c2nc(-c3ccccc3)nc(-c3cccc4oc5ccc(-c6cccc7c6oc6cc8ccccc8cc67)cc5c34)n2)cc1]

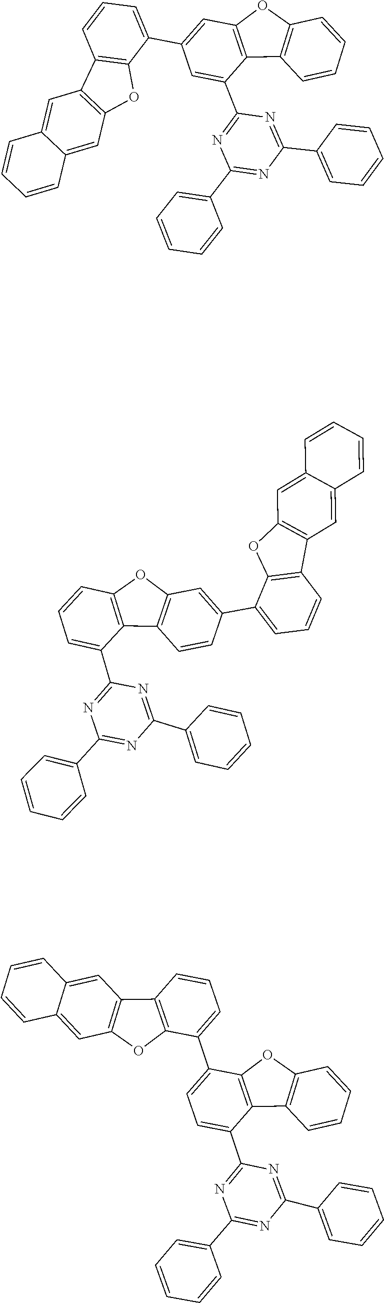 [SMILES: c1ccc(-c2nc(-c3ccccc3)nc(-c3cc(-c4cccc5c4oc4cc6ccccc6cc45)cc4oc5ccccc5c34)n2)cc1 ; c1ccc(-c2nc(-c3ccccc3)nc(-c3cccc4oc5cc(-c6cccc7c6oc6cc8ccccc8cc67)ccc5c34)n2)cc1 ; c1ccc(-c2nc(-c3ccccc3)nc(-c3ccc(-c4cccc5c4oc4cc6ccccc6cc45)c4oc5ccccc5c34)n2)cc1]

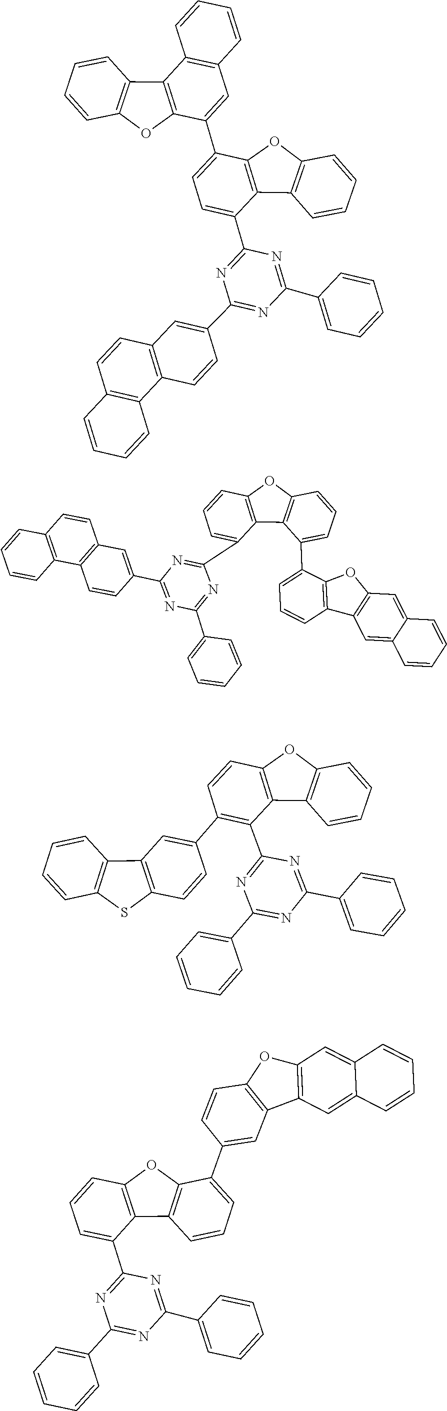 [SMILES: c1ccc(-c2nc(-c3ccc4c(ccc5ccccc54)c3)nc(-c3ccc(-c4cc5ccccc5c5c4oc4ccccc45)c4oc5ccccc5c34)n2)cc1 ; c1ccc(-c2nc(-c3ccc4c(ccc5ccccc54)c3)nc(-c3cccc4oc5cccc(-c6cccc7c6oc6cc8ccccc8cc67)c5c34)n2)cc1 ; c1ccc(-c2nc(-c3ccccc3)nc(-c3c(-c4ccc5sc6ccccc6c5c4)ccc4oc5ccccc5c34)n2)cc1 ; c1ccc(-c2nc(-c3ccccc3)nc(-c3cccc4oc5c(-c6ccc7oc8cc9ccccc9cc8c7c6)cccc5c34)n2)cc1]

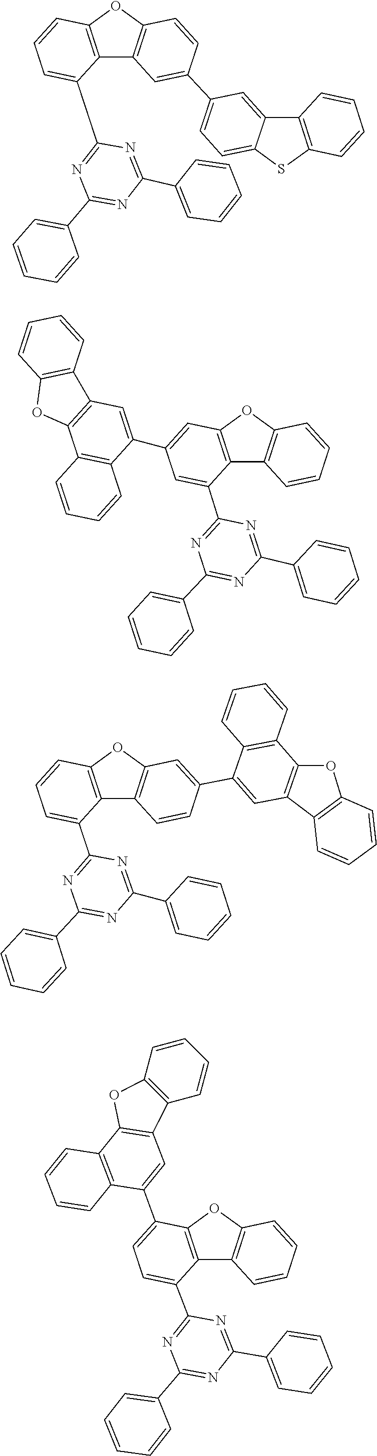

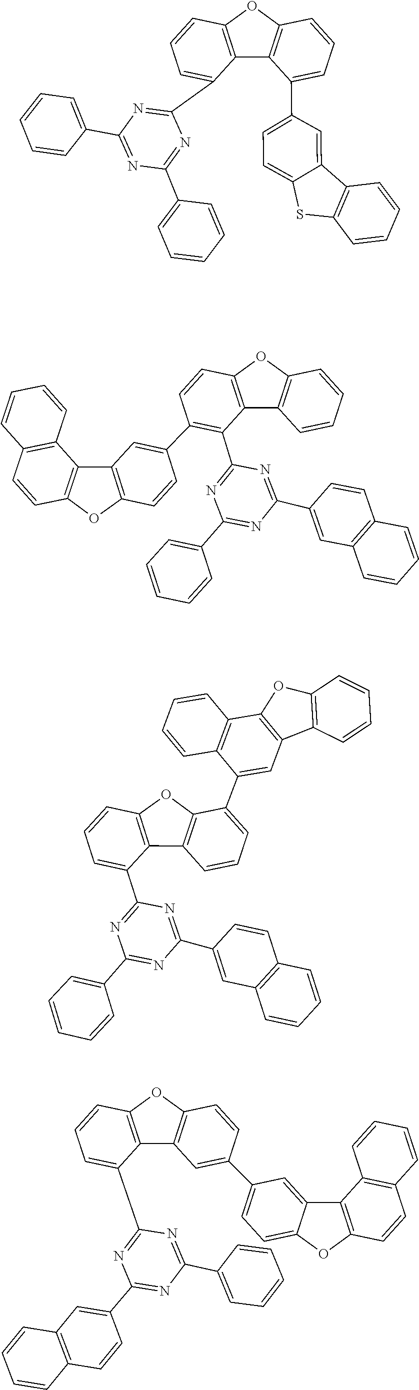 [SMILES: c1ccc(-c2nc(-c3ccccc3)nc(-c3cccc4oc5cccc(-c6ccc7sc8ccccc8c7c6)c5c34)n2)cc1 ; c1ccc(-c2nc(-c3ccc4ccccc4c3)nc(-c3c(-c4ccc5oc6ccc7ccccc7c6c5c4)ccc4oc5ccccc5c34)n2)cc1 ; c1ccc(-c2nc(-c3ccc4ccccc4c3)nc(-c3cccc4oc5c(-c6cc7c8ccccc8oc7c7ccccc67)cccc5c34)n2)cc1 ; c1ccc(-c2nc(-c3ccc4ccccc4c3)nc(-c3cccc4oc5ccc(-c6ccc7oc8ccc9ccccc9c8c7c6)cc5c34)n2)cc1]

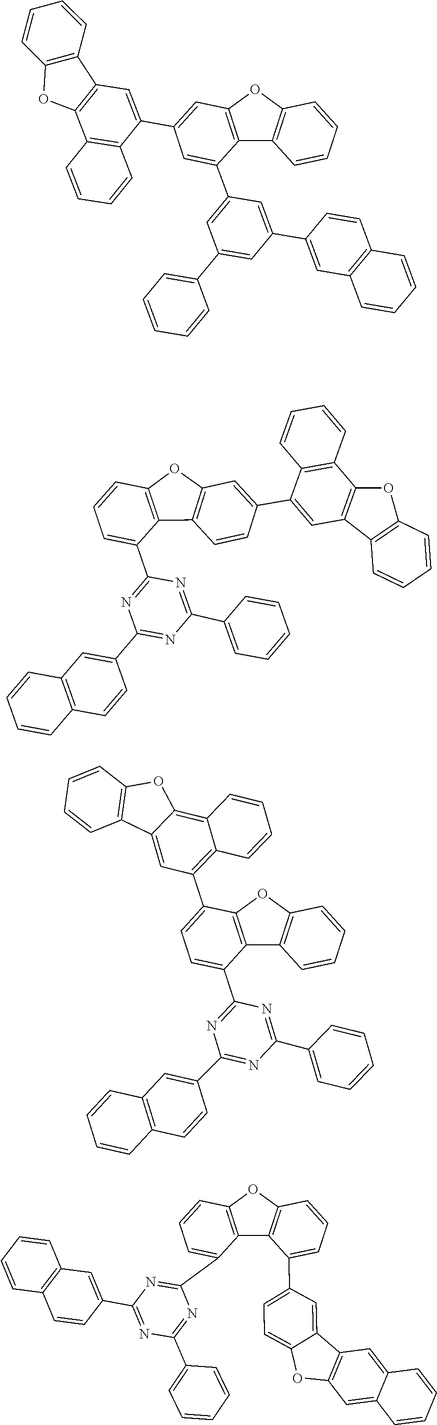 [SMILES: c1ccc(-c2cc(-c3ccc4ccccc4c3)cc(-c3cc(-c4cc5c6ccccc6oc5c5ccccc45)cc4oc5ccccc5c34)c2)cc1 ; c1ccc(-c2nc(-c3ccc4ccccc4c3)nc(-c3cccc4oc5cc(-c6cc7c8ccccc8oc7c7ccccc67)ccc5c34)n2)cc1 ; c1ccc(-c2nc(-c3ccc4ccccc4c3)nc(-c3ccc(-c4cc5c6ccccc6oc5c5ccccc45)c4oc5ccccc5c34)n2)cc1 ; c1ccc(-c2nc(-c3ccc4ccccc4c3)nc(-c3cccc4oc5cccc(-c6ccc7oc8cc9ccccc9cc8c7c6)c5c34)n2)cc1]

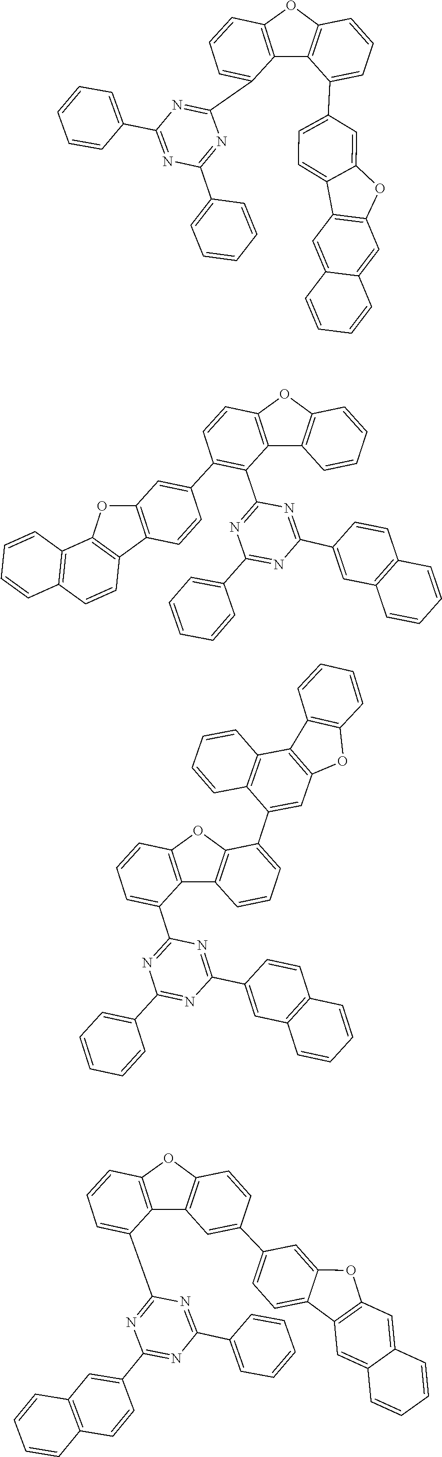 [SMILES: c1ccc(-c2nc(-c3ccccc3)nc(-c3cccc4oc5cccc(-c6ccc7c(c6)oc6cc8ccccc8cc67)c5c34)n2)cc1 ; c1ccc(-c2nc(-c3ccc4ccccc4c3)nc(-c3c(-c4ccc5c(c4)oc4c6ccccc6ccc54)ccc4oc5ccccc5c34)n2)cc1 ; c1ccc(-c2nc(-c3ccc4ccccc4c3)nc(-c3cccc4oc5c(-c6cc7oc8ccccc8c7c7ccccc67)cccc5c34)n2)cc1 ; c1ccc(-c2nc(-c3ccc4ccccc4c3)nc(-c3cccc4oc5ccc(-c6ccc7c(c6)oc6cc8ccccc8cc67)cc5c34)n2)cc1]

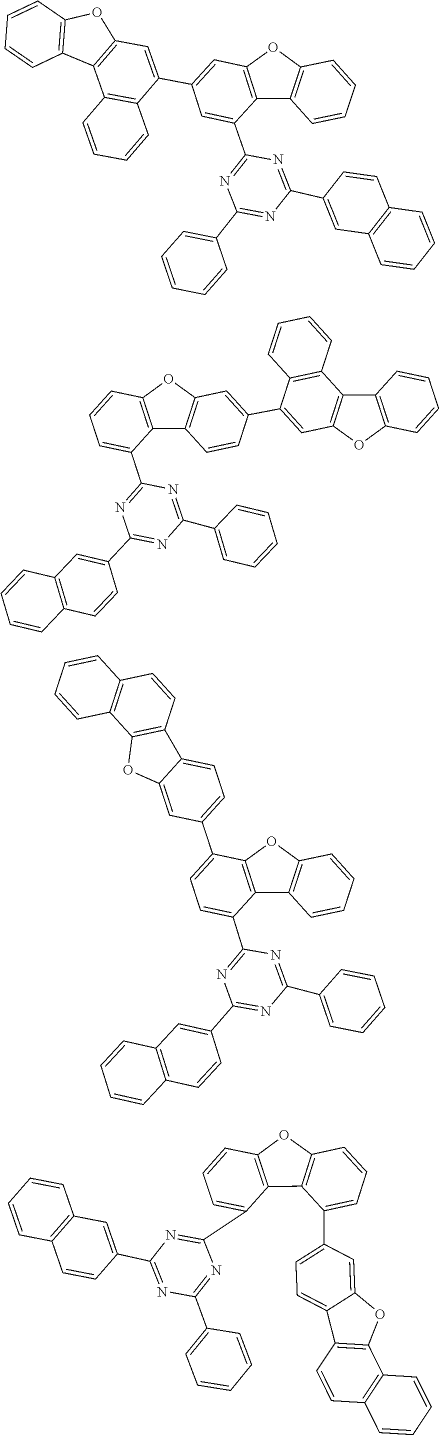

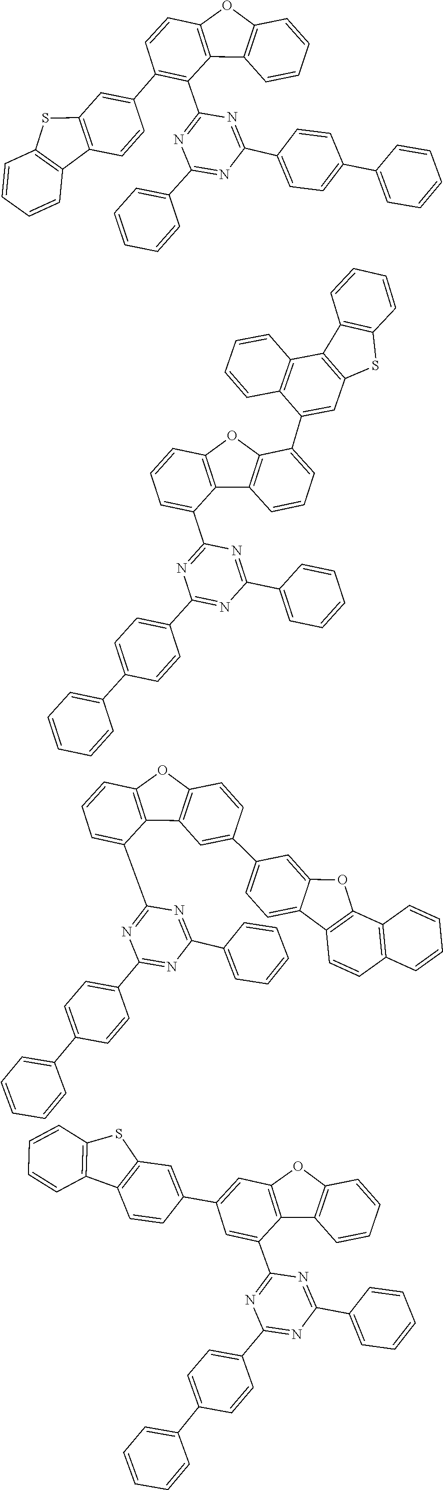

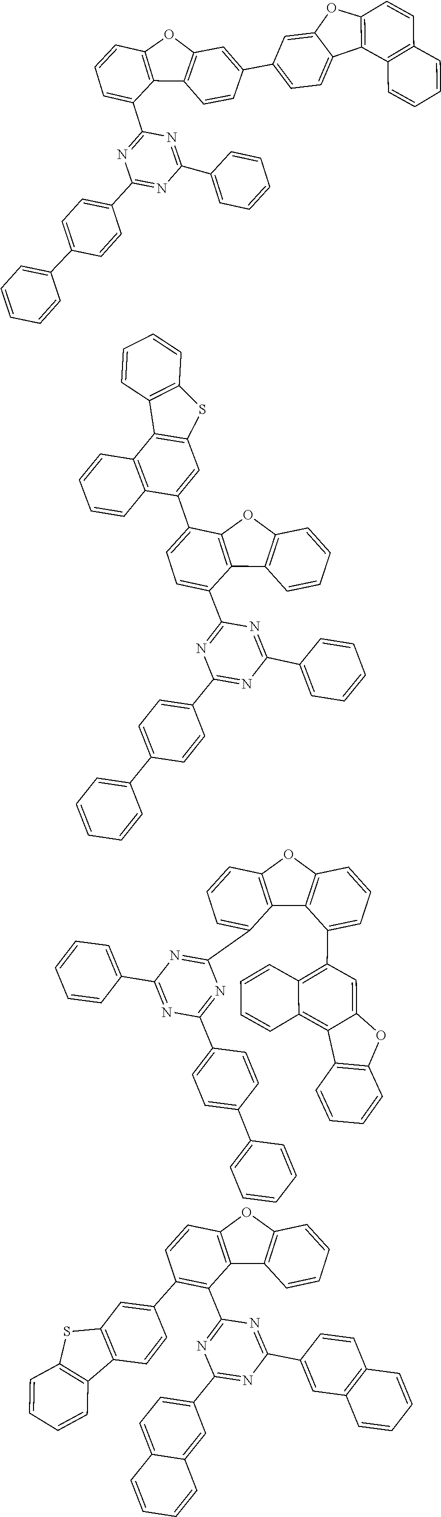

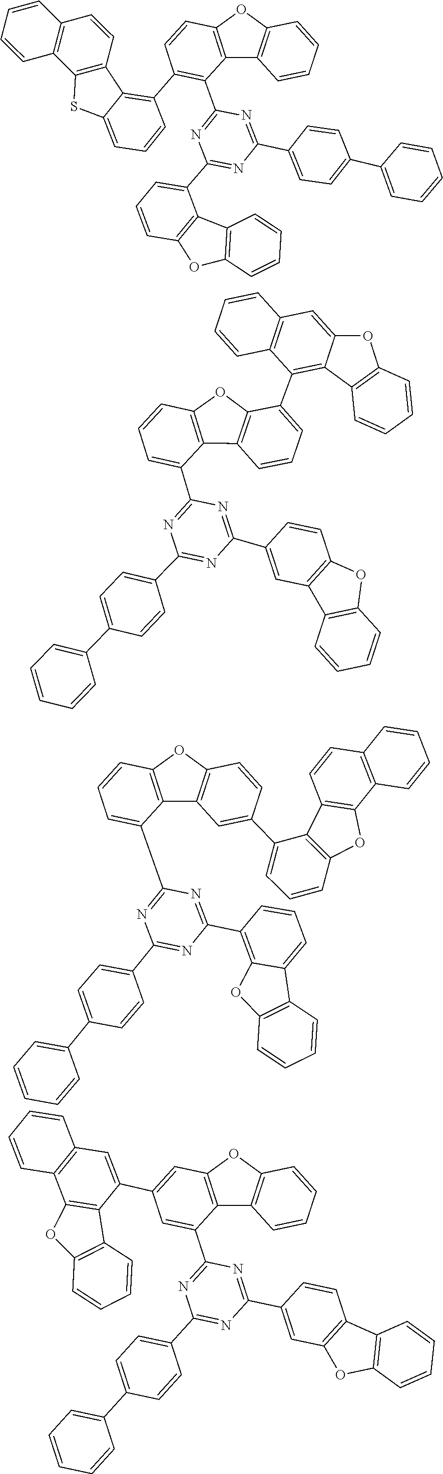

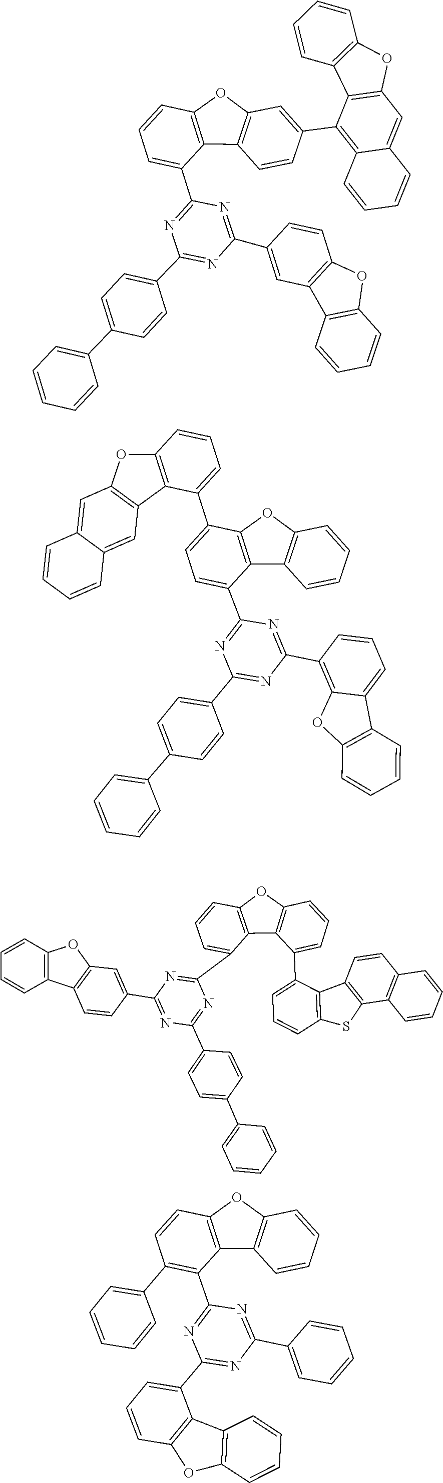

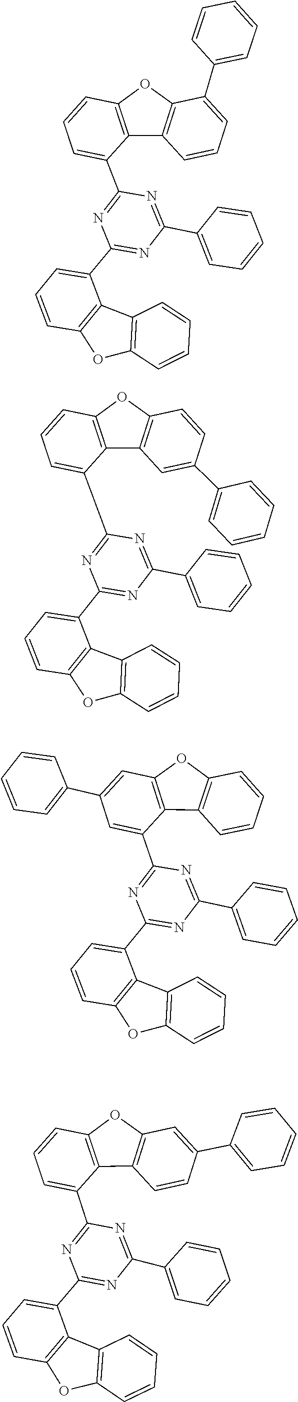

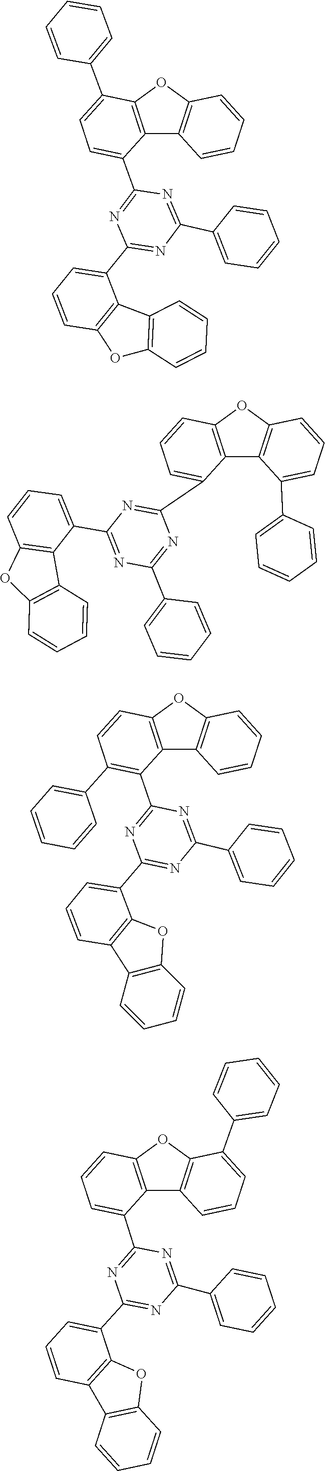

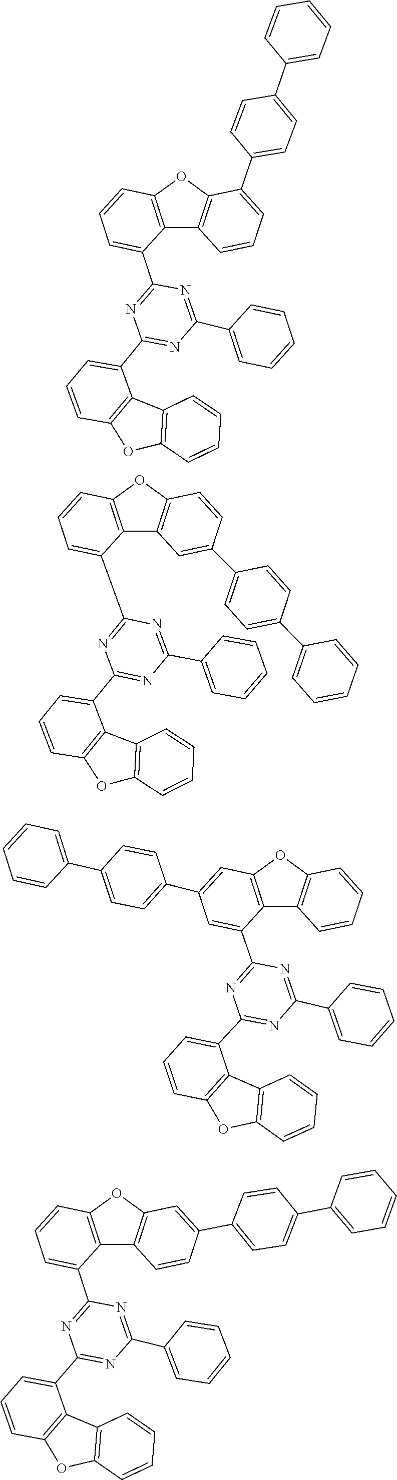

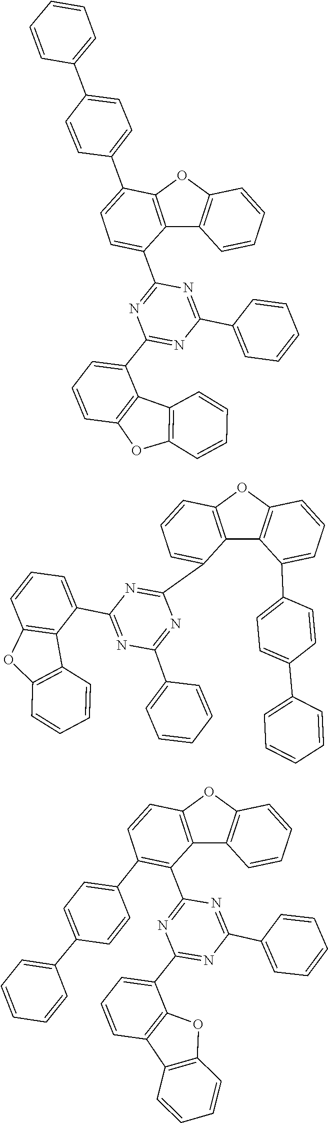

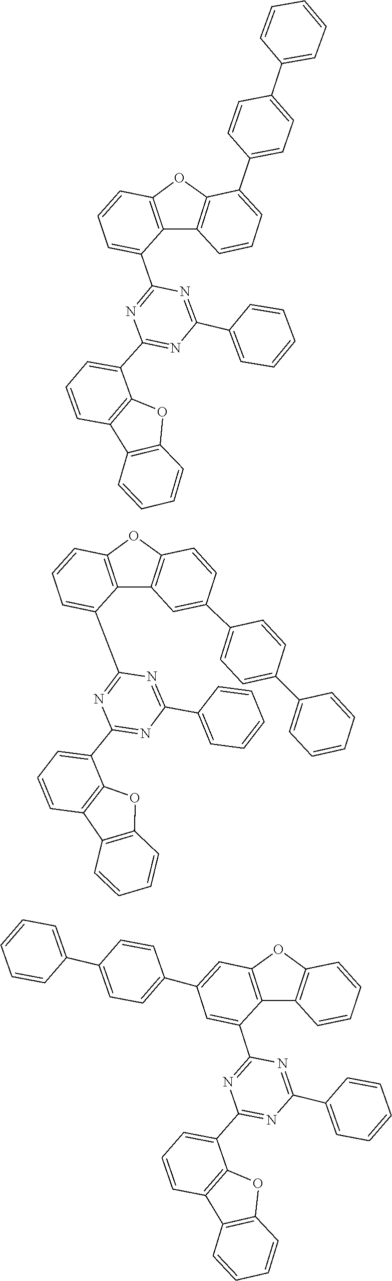

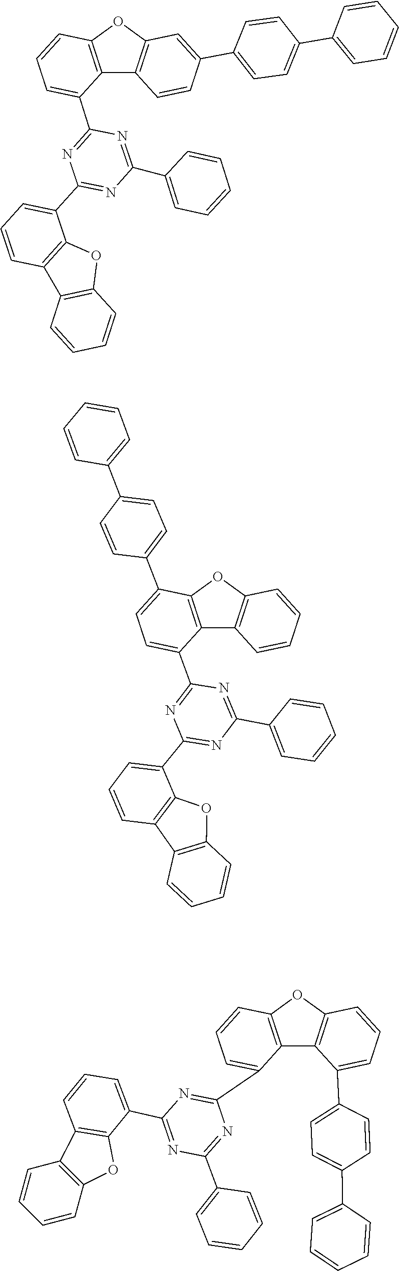 [SMILES: c1ccc(-c2ccc(-c3ccc4c(c3)oc3cccc(-c5nc(-c6ccccc6)nc(-c6cccc7c6oc6ccccc67)n5)c34)cc2)cc1 ; c1ccc(-c2ccc(-c3ccc(-c4nc(-c5ccccc5)nc(-c5cccc6c5oc5ccccc56)n4)c4c3oc3ccccc34)cc2)cc1 ; c1ccc(-c2ccc(-c3cccc4oc5cccc(-c6nc(-c7ccccc7)nc(-c7cccc8c7oc7ccccc78)n6)c5c34)cc2)cc1]

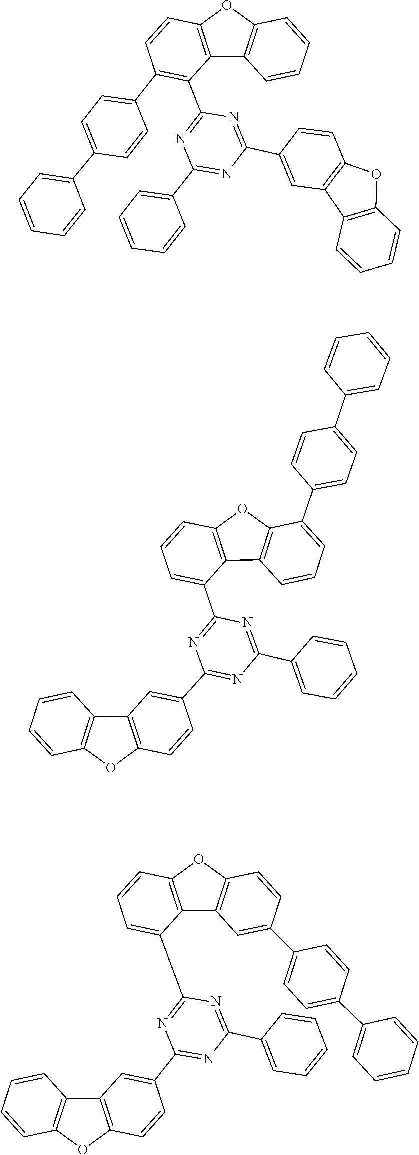 [SMILES: c1ccc(-c2ccc(-c3ccc4oc5ccccc5c4c3-c3nc(-c4ccccc4)nc(-c4ccc5oc6ccccc6c5c4)n3)cc2)cc1 ; c1ccc(-c2ccc(-c3cccc4c3oc3cccc(-c5nc(-c6ccccc6)nc(-c6ccc7oc8ccccc8c7c6)n5)c34)cc2)cc1 ; c1ccc(-c2ccc(-c3ccc4oc5cccc(-c6nc(-c7ccccc7)nc(-c7ccc8oc9ccccc9c8c7)n6)c5c4c3)cc2)cc1]

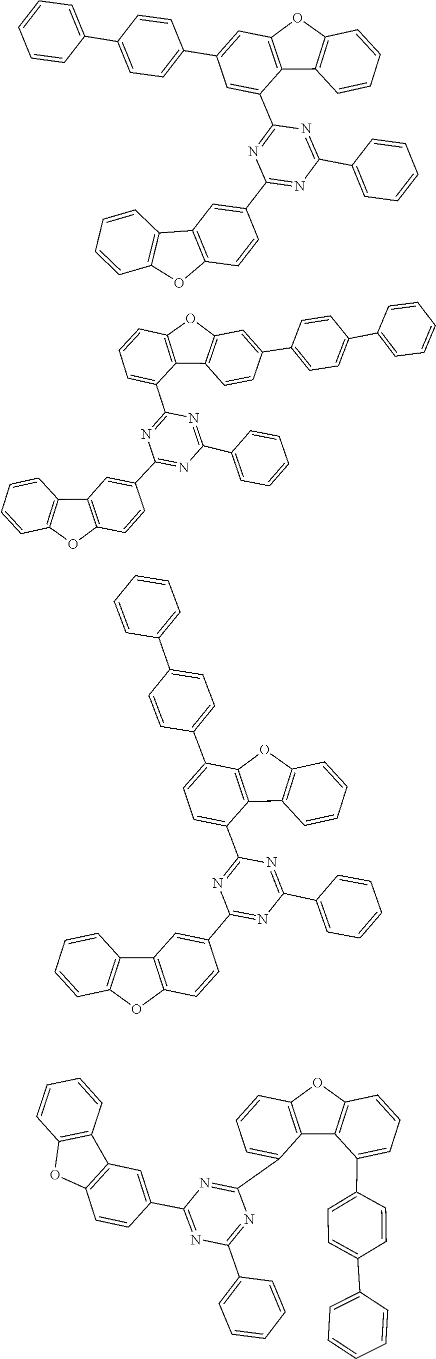

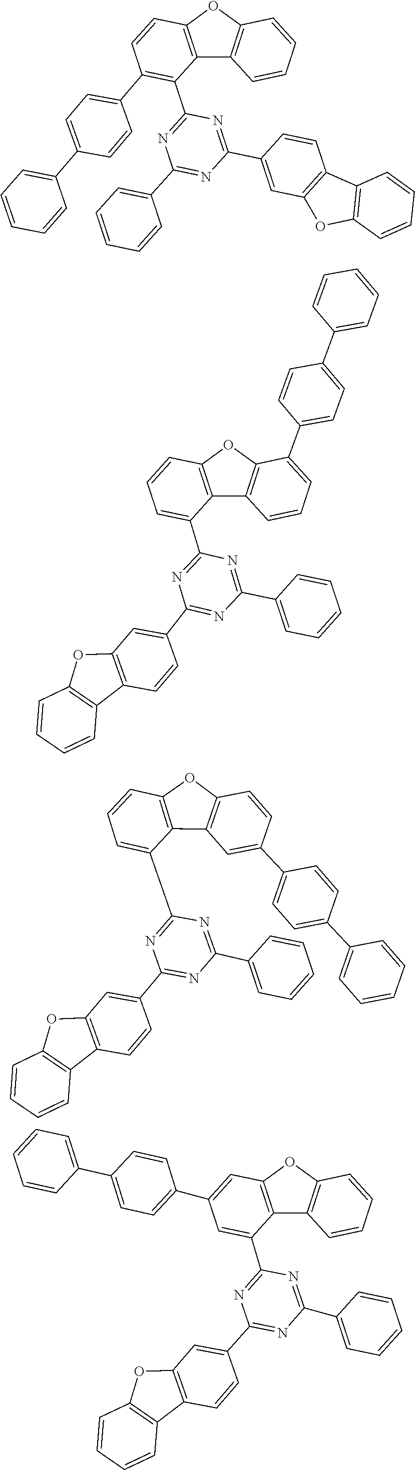

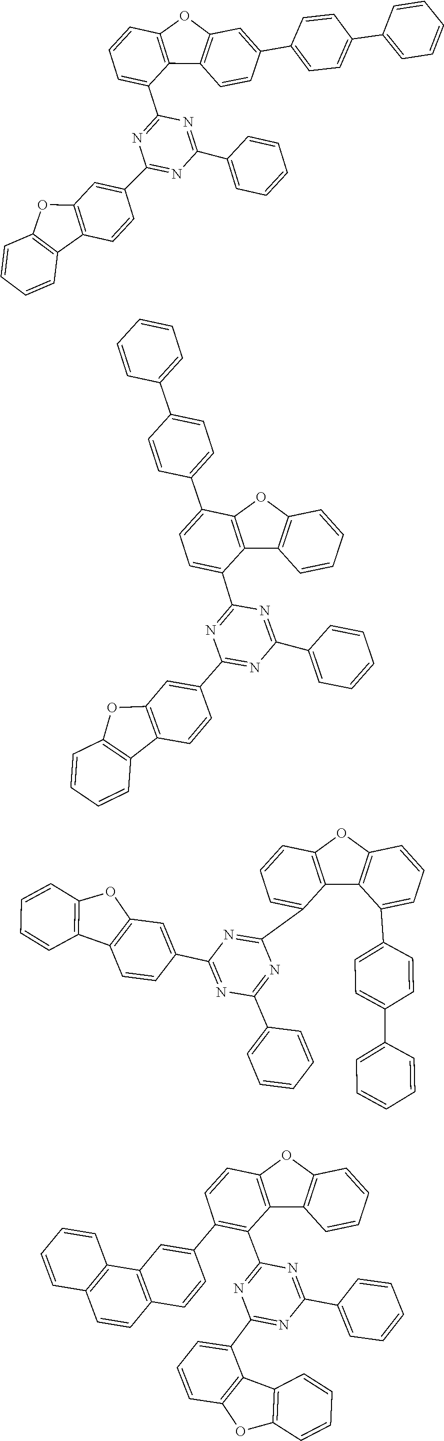 [SMILES: c1ccc(-c2ccc(-c3ccc4c(c3)oc3cccc(-c5nc(-c6ccccc6)nc(-c6ccc7c(c6)oc6ccccc67)n5)c34)cc2)cc1 ; c1ccc(-c2ccc(-c3ccc(-c4nc(-c5ccccc5)nc(-c5ccc6c(c5)oc5ccccc56)n4)c4c3oc3ccccc34)cc2)cc1 ; c1ccc(-c2ccc(-c3cccc4oc5cccc(-c6nc(-c7ccccc7)nc(-c7ccc8c(c7)oc7ccccc78)n6)c5c34)cc2)cc1 ; c1ccc(-c2nc(-c3cccc4oc5ccccc5c34)nc(-c3c(-c4ccc5ccc6ccccc6c5c4)ccc4oc5ccccc5c34)n2)cc1]

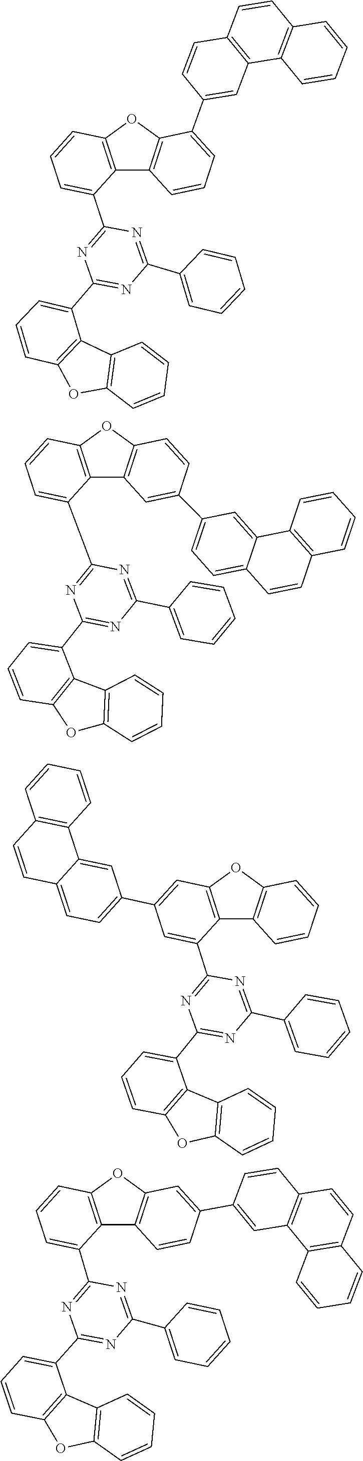

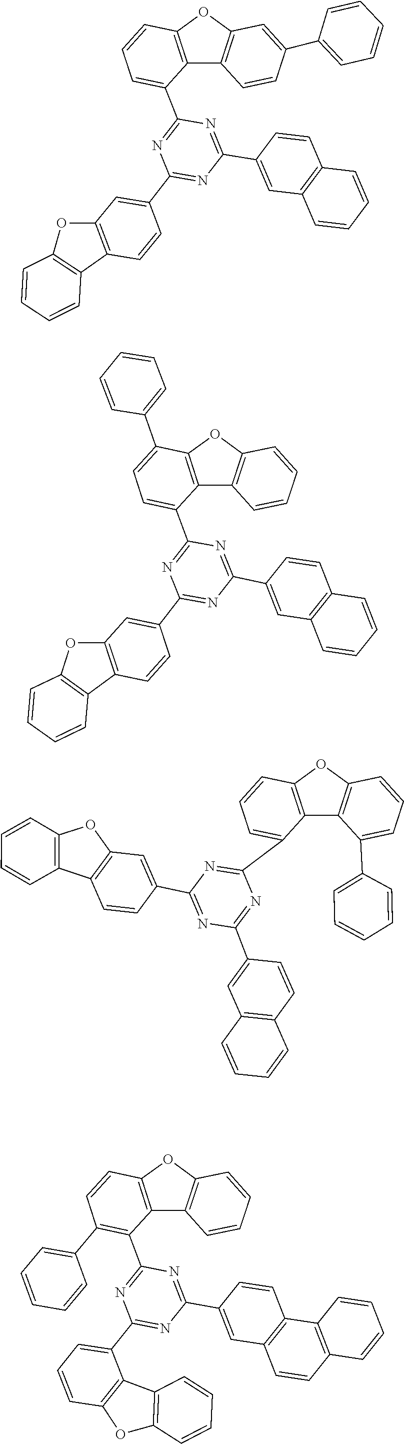 [SMILES: c1ccc(-c2ccc3c(c2)oc2cccc(-c4nc(-c5ccc6ccccc6c5)nc(-c5ccc6c(c5)oc5ccccc56)n4)c23)cc1 ; c1ccc(-c2ccc(-c3nc(-c4ccc5ccccc5c4)nc(-c4ccc5c(c4)oc4ccccc45)n3)c3c2oc2ccccc23)cc1 ; c1ccc(-c2cccc3oc4cccc(-c5nc(-c6ccc7ccccc7c6)nc(-c6ccc7c(c6)oc6ccccc67)n5)c4c23)cc1 ; c1ccc(-c2ccc3oc4ccccc4c3c2-c2nc(-c3ccc4c(ccc5ccccc54)c3)nc(-c3cccc4oc5ccccc5c34)n2)cc1]

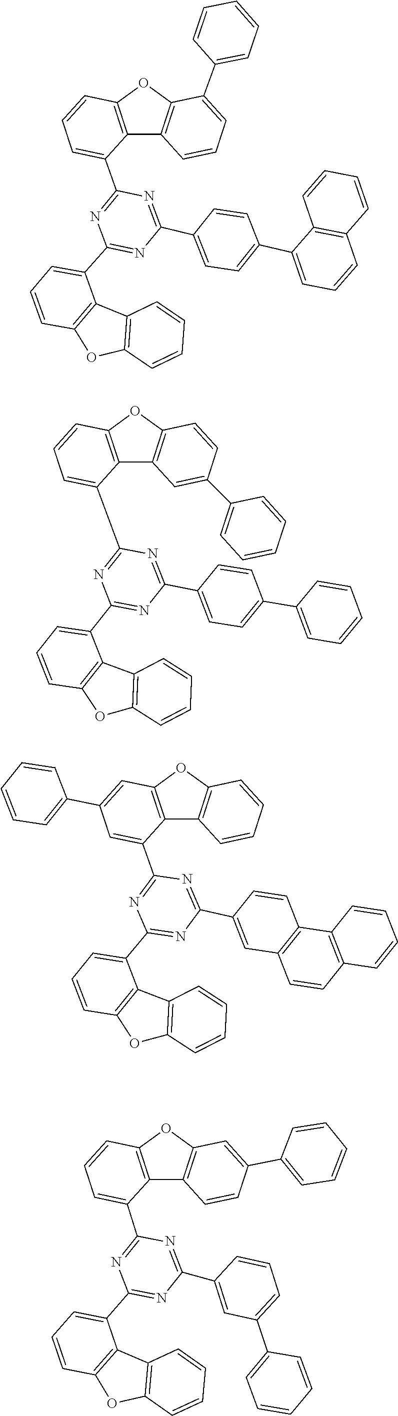

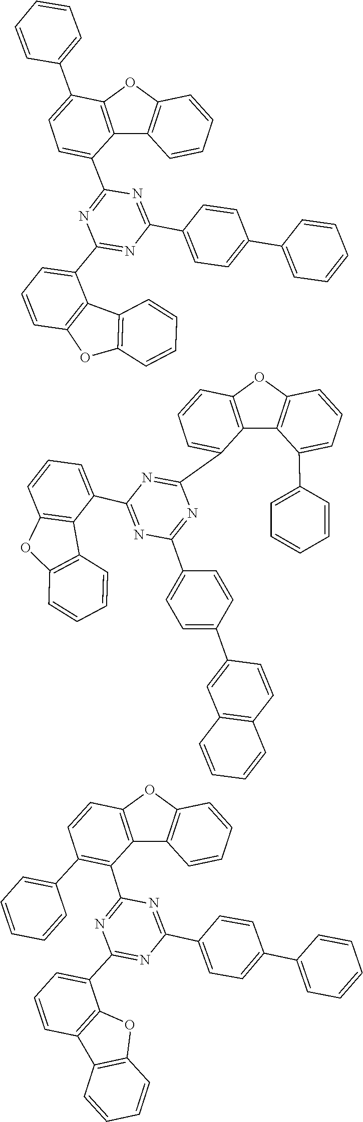

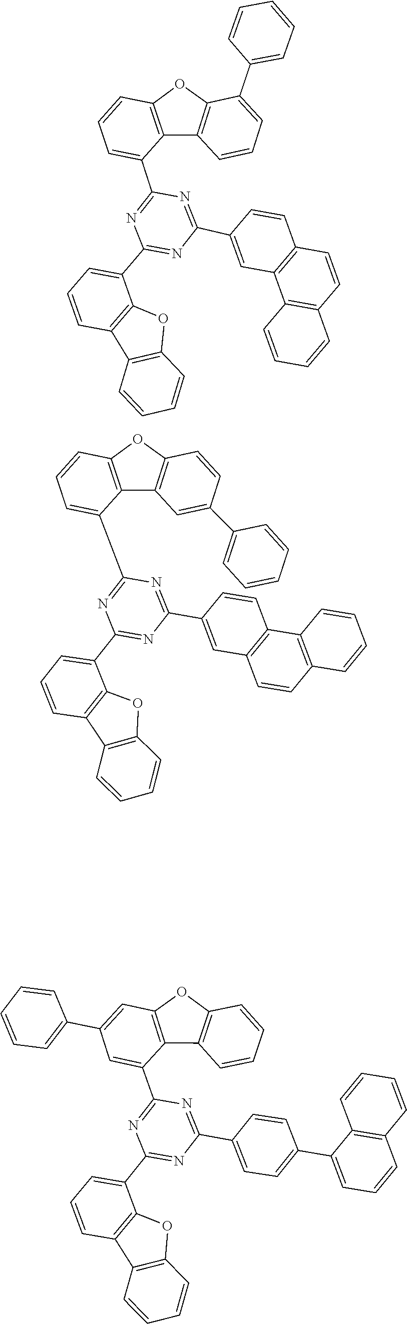

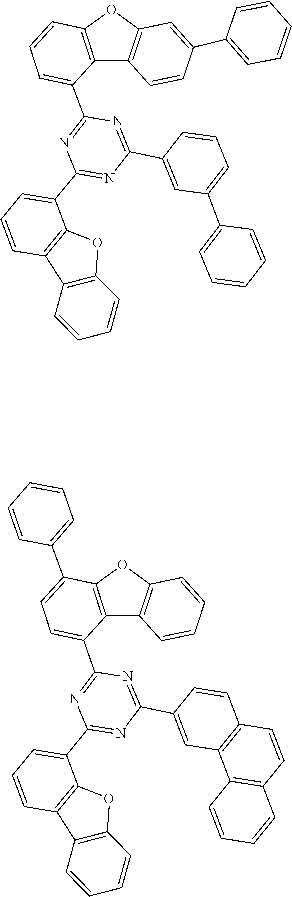 [SMILES: c1ccc(-c2cccc(-c3nc(-c4cccc5c4oc4ccccc45)nc(-c4cccc5oc6cc(-c7ccccc7)ccc6c45)n3)c2)cc1 ; c1ccc(-c2ccc(-c3nc(-c4ccc5ccc6ccccc6c5c4)nc(-c4cccc5c4oc4ccccc45)n3)c3c2oc2ccccc23)cc1]

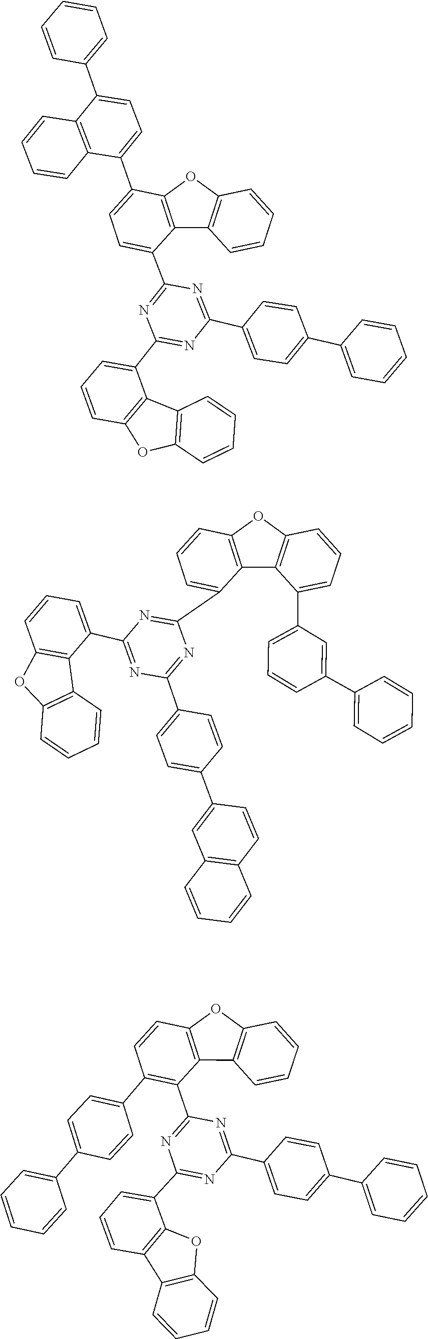 [SMILES: c1ccc(-c2ccc(-c3nc(-c4cccc5oc6ccccc6c45)nc(-c4ccc(-c5ccc(-c6ccccc6)c6ccccc56)c5oc6ccccc6c45)n3)cc2)cc1 ; c1ccc(-c2cccc(-c3cccc4oc5cccc(-c6nc(-c7ccc(-c8ccc9ccccc9c8)cc7)nc(-c7cccc8oc9ccccc9c78)n6)c5c34)c2)cc1 ; c1ccc(-c2ccc(-c3nc(-c4cccc5c4oc4ccccc45)nc(-c4c(-c5ccc(-c6ccccc6)cc5)ccc5oc6ccccc6c45)n3)cc2)cc1]

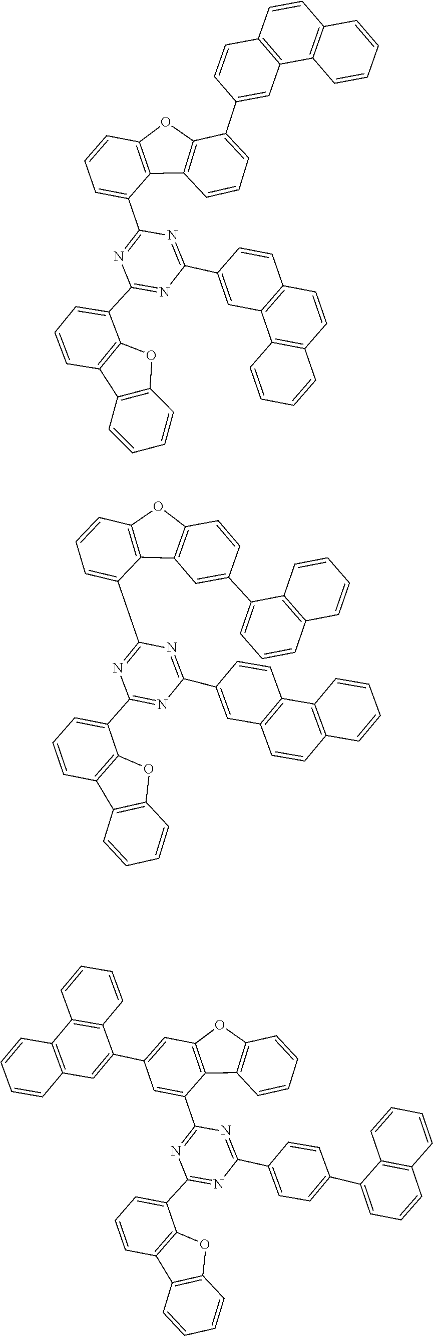 [SMILES: c1ccc2c(c1)ccc1ccc(-c3nc(-c4cccc5c4oc4ccccc45)nc(-c4cccc5oc6c(-c7ccc8ccc9ccccc9c8c7)cccc6c45)n3)cc12 ; c1ccc2c(-c3ccc4oc5cccc(-c6nc(-c7ccc8c(ccc9ccccc98)c7)nc(-c7cccc8c7oc7ccccc78)n6)c5c4c3)cccc2c1 ; c1ccc2c(-c3ccc(-c4nc(-c5cccc6c5oc5ccccc56)nc(-c5cc(-c6cc7ccccc7c7ccccc67)cc6oc7ccccc7c56)n4)cc3)cccc2c1]

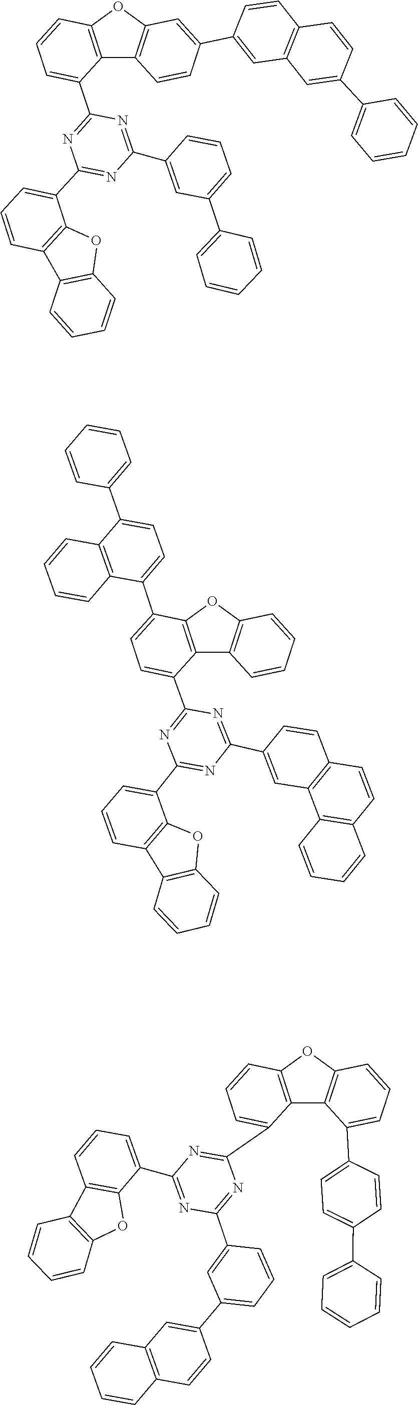 [SMILES: c1ccc(-c2cccc(-c3nc(-c4cccc5c4oc4ccccc45)nc(-c4cccc5oc6cc(-c7ccc8ccc(-c9ccccc9)cc8c7)ccc6c45)n3)c2)cc1 ; c1ccc(-c2ccc(-c3ccc(-c4nc(-c5ccc6ccc7ccccc7c6c5)nc(-c5cccc6c5oc5ccccc56)n4)c4c3oc3ccccc34)c3ccccc23)cc1 ; c1ccc(-c2ccc(-c3cccc4oc5cccc(-c6nc(-c7cccc(-c8ccc9ccccc9c8)c7)nc(-c7cccc8c7oc7ccccc78)n6)c5c34)cc2)cc1]

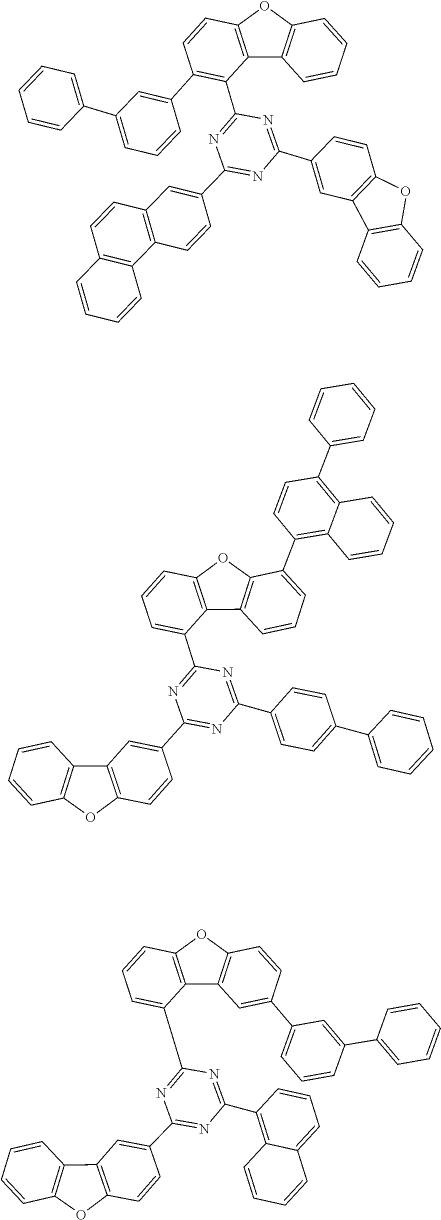 [SMILES: c1ccc(-c2cccc(-c3ccc4oc5ccccc5c4c3-c3nc(-c4ccc5c(ccc6ccccc65)c4)nc(-c4ccc5oc6ccccc6c5c4)n3)c2)cc1 ; c1ccc(-c2ccc(-c3nc(-c4ccc5oc6ccccc6c5c4)nc(-c4cccc5oc6c(-c7ccc(-c8ccccc8)c8ccccc78)cccc6c45)n3)cc2)cc1 ; c1ccc(-c2cccc(-c3ccc4oc5cccc(-c6nc(-c7ccc8oc9ccccc9c8c7)nc(-c7cccc8ccccc78)n6)c5c4c3)c2)cc1]

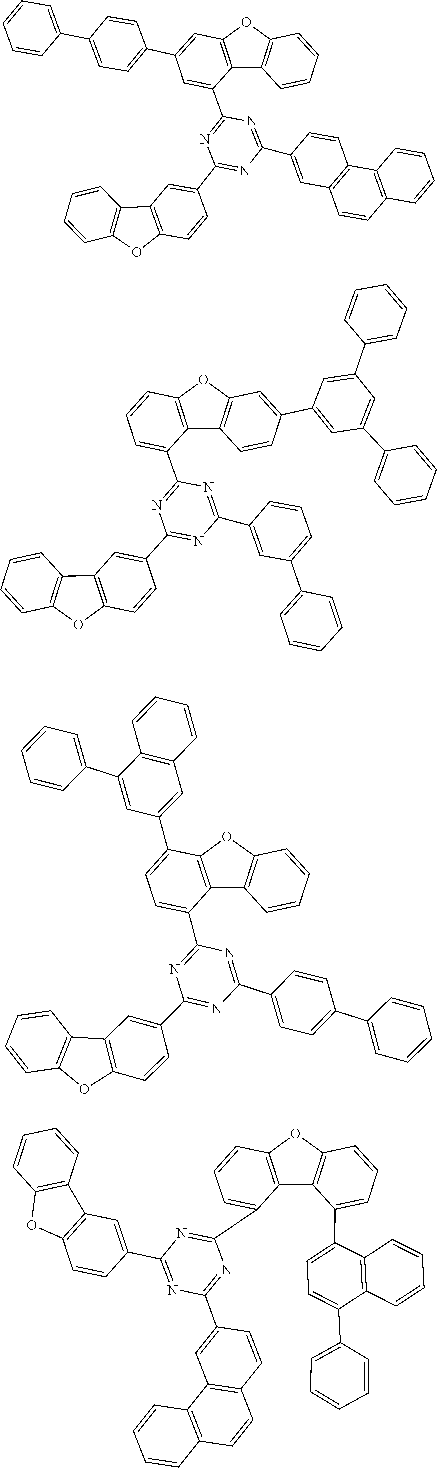 [SMILES: c1ccc(-c2ccc(-c3cc(-c4nc(-c5ccc6c(ccc7ccccc76)c5)nc(-c5ccc6oc7ccccc7c6c5)n4)c4c(c3)oc3ccccc34)cc2)cc1 ; c1ccc(-c2cc(-c3ccccc3)cc(-c3ccc4c(c3)oc3cccc(-c5nc(-c6cccc(-c7ccccc7)c6)nc(-c6ccc7oc8ccccc8c7c6)n5)c34)c2)cc1 ; c1ccc(-c2ccc(-c3nc(-c4ccc5oc6ccccc6c5c4)nc(-c4ccc(-c5cc(-c6ccccc6)c6ccccc6c5)c5oc6ccccc6c45)n3)cc2)cc1 ; c1ccc(-c2ccc(-c3cccc4oc5cccc(-c6nc(-c7ccc8ccc9ccccc9c8c7)nc(-c7ccc8oc9ccccc9c8c7)n6)c5c34)c3ccccc23)cc1]

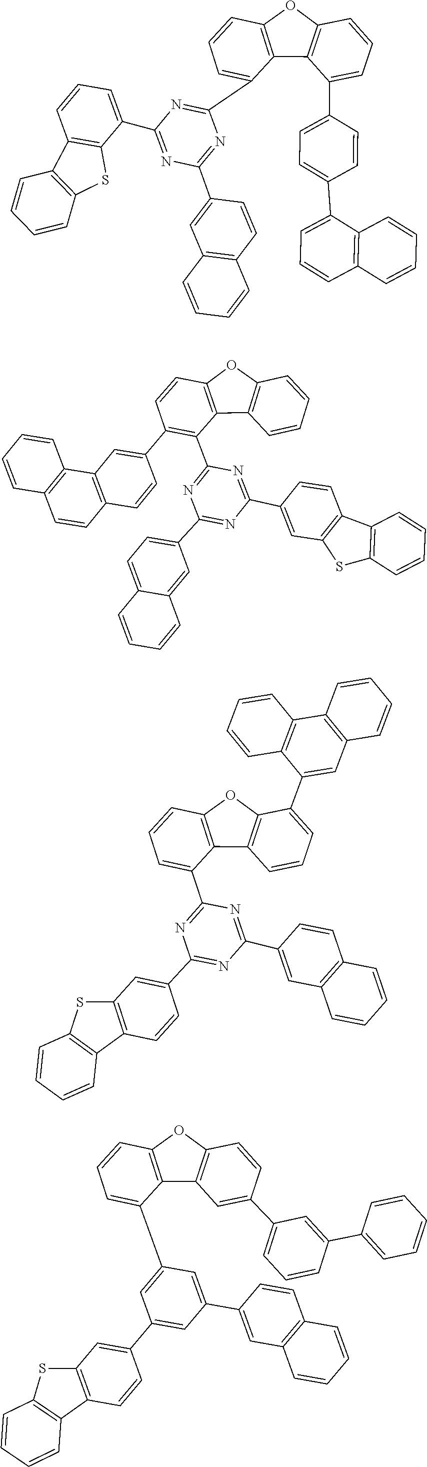 [SMILES: c1ccc2cc(-c3nc(-c4cccc5c4sc4ccccc45)nc(-c4cccc5oc6cccc(-c7ccc(-c8cccc9ccccc89)cc7)c6c45)n3)ccc2c1 ; c1ccc2cc(-c3nc(-c4ccc5c(c4)sc4ccccc45)nc(-c4c(-c5ccc6ccc7ccccc7c6c5)ccc5oc6ccccc6c45)n3)ccc2c1 ; c1ccc2cc(-c3nc(-c4ccc5c(c4)sc4ccccc45)nc(-c4cccc5oc6c(-c7cc8ccccc8c8ccccc78)cccc6c45)n3)ccc2c1 ; c1ccc(-c2cccc(-c3ccc4oc5cccc(-c6cc(-c7ccc8ccccc8c7)cc(-c7ccc8c(c7)sc7ccccc78)c6)c5c4c3)c2)cc1]

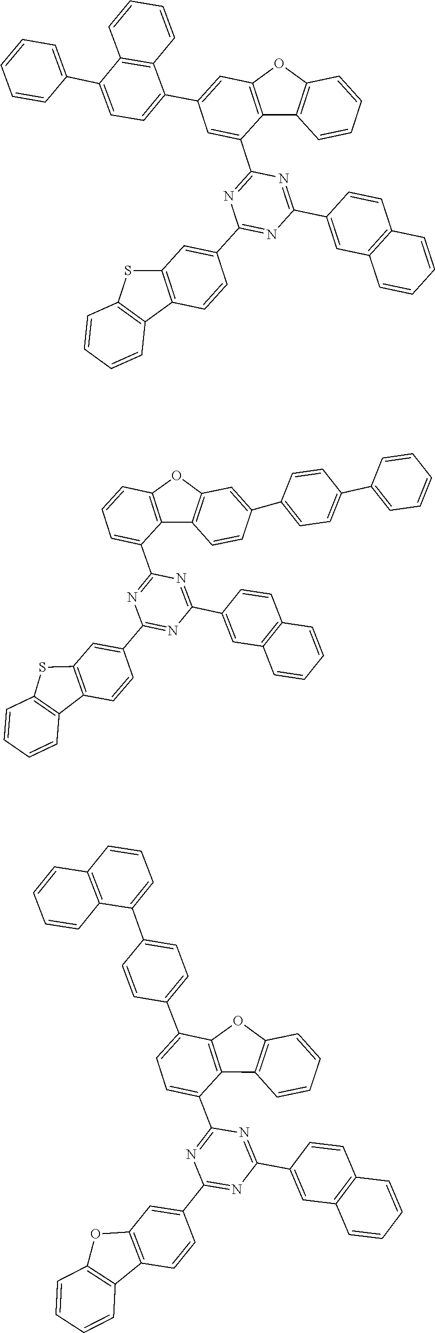 [SMILES: c1ccc(-c2ccc(-c3cc(-c4nc(-c5ccc6ccccc6c5)nc(-c5ccc6c(c5)sc5ccccc56)n4)c4c(c3)oc3ccccc34)c3ccccc23)cc1 ; c1ccc(-c2ccc(-c3ccc4c(c3)oc3cccc(-c5nc(-c6ccc7ccccc7c6)nc(-c6ccc7c(c6)sc6ccccc67)n5)c34)cc2)cc1 ; c1ccc2cc(-c3nc(-c4ccc5c(c4)oc4ccccc45)nc(-c4ccc(-c5ccc(-c6cccc7ccccc67)cc5)c5oc6ccccc6c45)n3)ccc2c1]

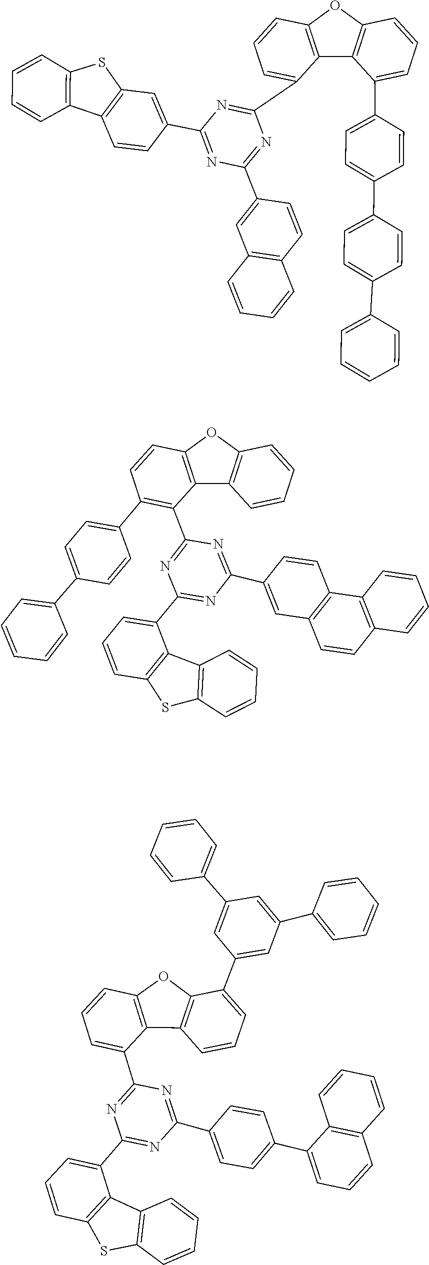 [SMILES: c1ccc(-c2ccc(-c3ccc(-c4cccc5oc6cccc(-c7nc(-c8ccc9ccccc9c8)nc(-c8ccc9c(c8)sc8ccccc89)n7)c6c45)cc3)cc2)cc1 ; c1ccc(-c2ccc(-c3ccc4oc5ccccc5c4c3-c3nc(-c4ccc5c(ccc6ccccc65)c4)nc(-c4cccc5sc6ccccc6c45)n3)cc2)cc1 ; c1ccc(-c2cc(-c3ccccc3)cc(-c3cccc4c3oc3cccc(-c5nc(-c6ccc(-c7cccc8ccccc78)cc6)nc(-c6cccc7sc8ccccc8c67)n5)c34)c2)cc1]

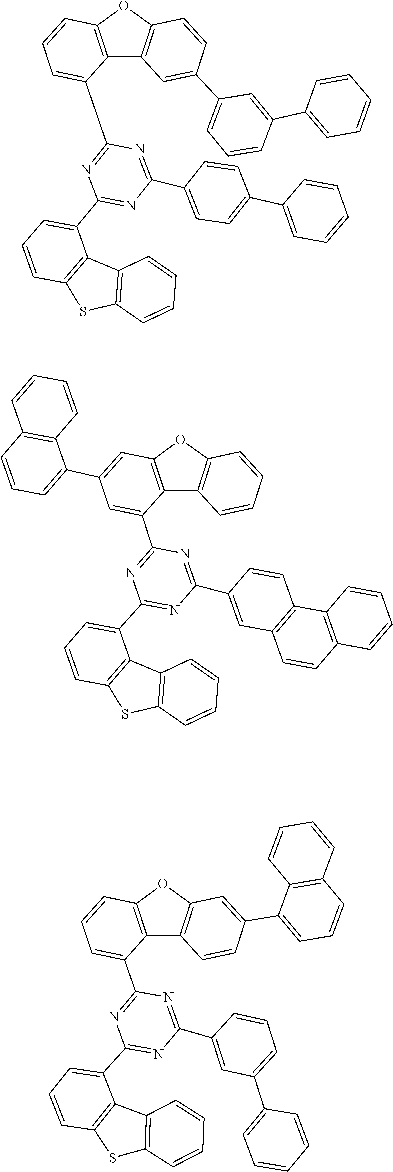 [SMILES: c1ccc(-c2ccc(-c3nc(-c4cccc5oc6ccc(-c7cccc(-c8ccccc8)c7)cc6c45)nc(-c4cccc5sc6ccccc6c45)n3)cc2)cc1 ; c1ccc2c(-c3cc(-c4nc(-c5ccc6c(ccc7ccccc76)c5)nc(-c5cccc6sc7ccccc7c56)n4)c4c(c3)oc3ccccc34)cccc2c1 ; c1ccc(-c2cccc(-c3nc(-c4cccc5oc6cc(-c7cccc8ccccc78)ccc6c45)nc(-c4cccc5sc6ccccc6c45)n3)c2)cc1]

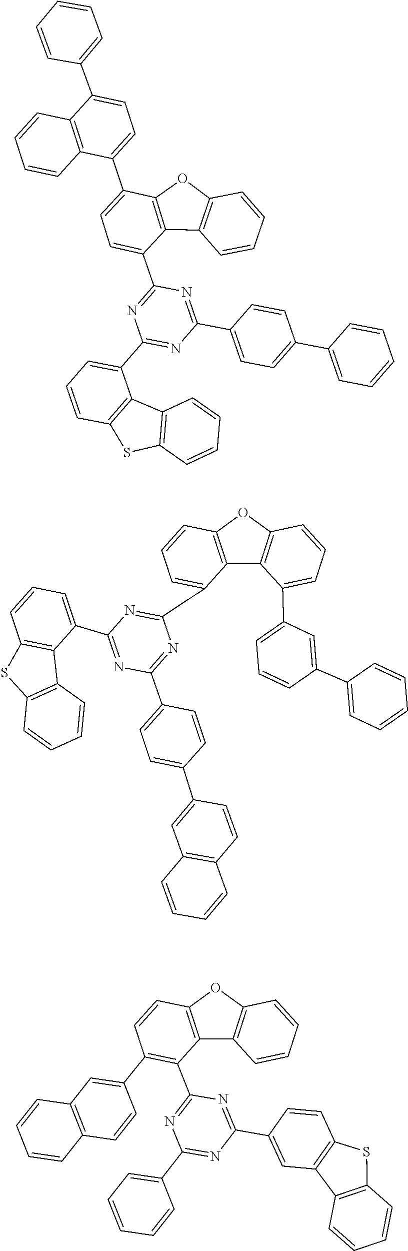 [SMILES: c1ccc(-c2ccc(-c3nc(-c4cccc5sc6ccccc6c45)nc(-c4ccc(-c5ccc(-c6ccccc6)c6ccccc56)c5oc6ccccc6c45)n3)cc2)cc1 ; c1ccc(-c2cccc(-c3cccc4oc5cccc(-c6nc(-c7ccc(-c8ccc9ccccc9c8)cc7)nc(-c7cccc8sc9ccccc9c78)n6)c5c34)c2)cc1 ; c1ccc(-c2nc(-c3ccc4sc5ccccc5c4c3)nc(-c3c(-c4ccc5ccccc5c4)ccc4oc5ccccc5c34)n2)cc1]

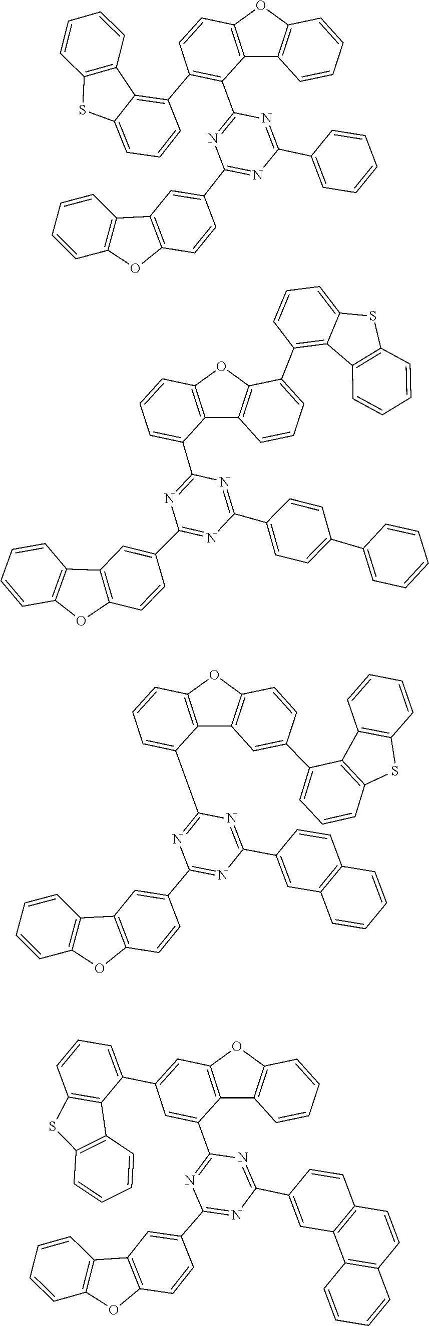 [SMILES: c1ccc(-c2nc(-c3ccc4oc5ccccc5c4c3)nc(-c3c(-c4cccc5sc6ccccc6c45)ccc4oc5ccccc5c34)n2)cc1 ; c1ccc(-c2ccc(-c3nc(-c4ccc5oc6ccccc6c5c4)nc(-c4cccc5oc6c(-c7cccc8sc9ccccc9c78)cccc6c45)n3)cc2)cc1 ; c1ccc2cc(-c3nc(-c4ccc5oc6ccccc6c5c4)nc(-c4cccc5oc6ccc(-c7cccc8sc9ccccc9c78)cc6c45)n3)ccc2c1 ; c1ccc2c(c1)ccc1ccc(-c3nc(-c4ccc5oc6ccccc6c5c4)nc(-c4cc(-c5cccc6sc7ccccc7c56)cc5oc6ccccc6c45)n3)cc12]

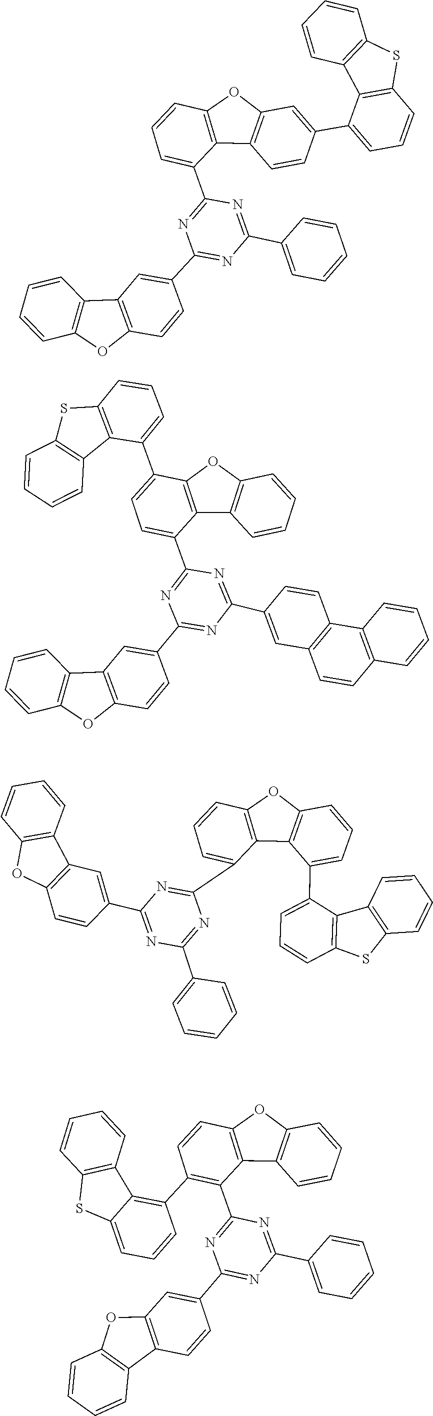

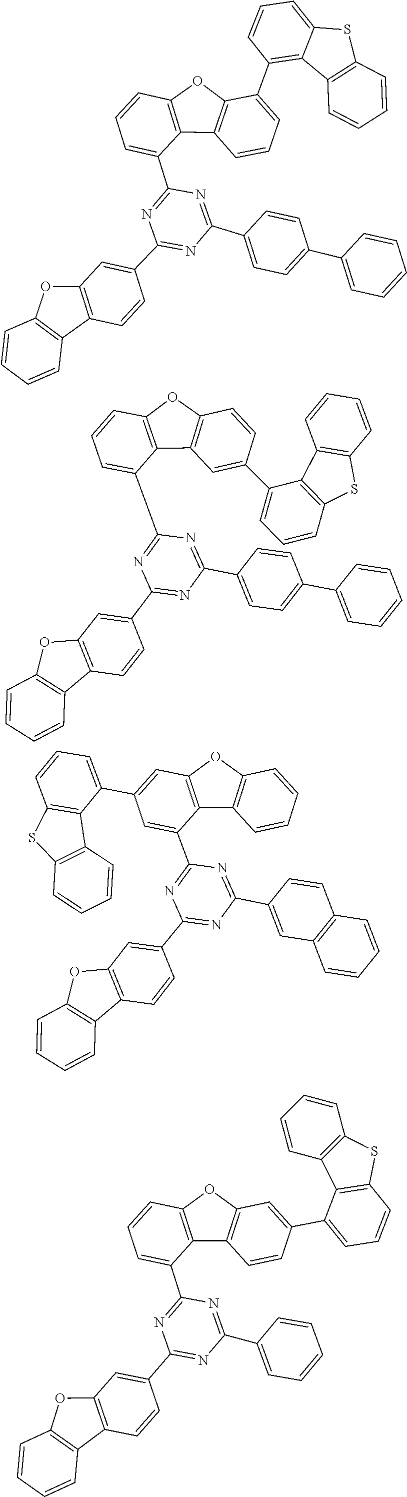

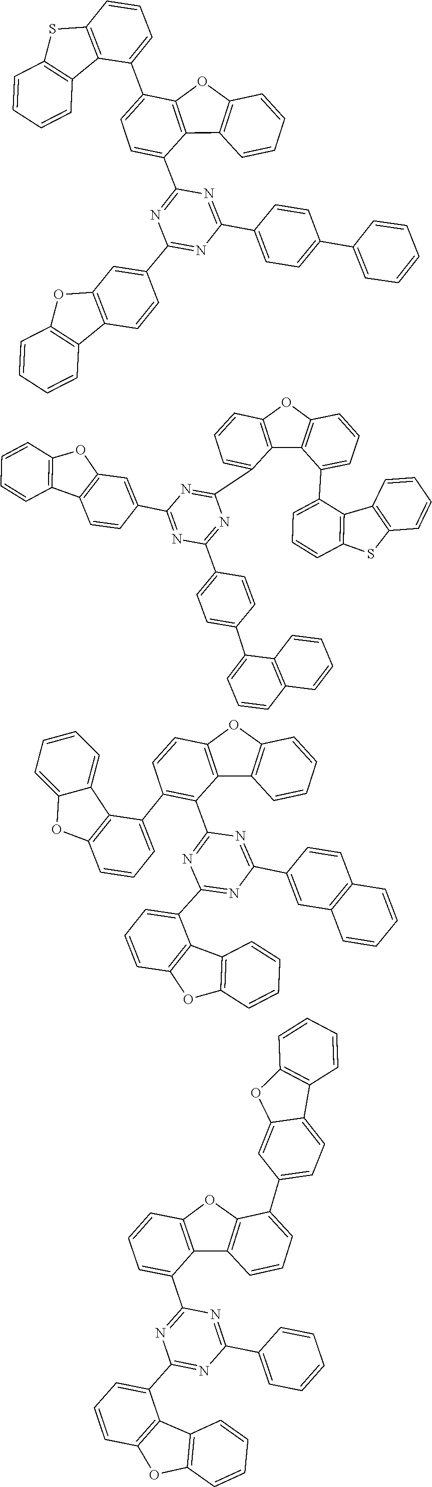

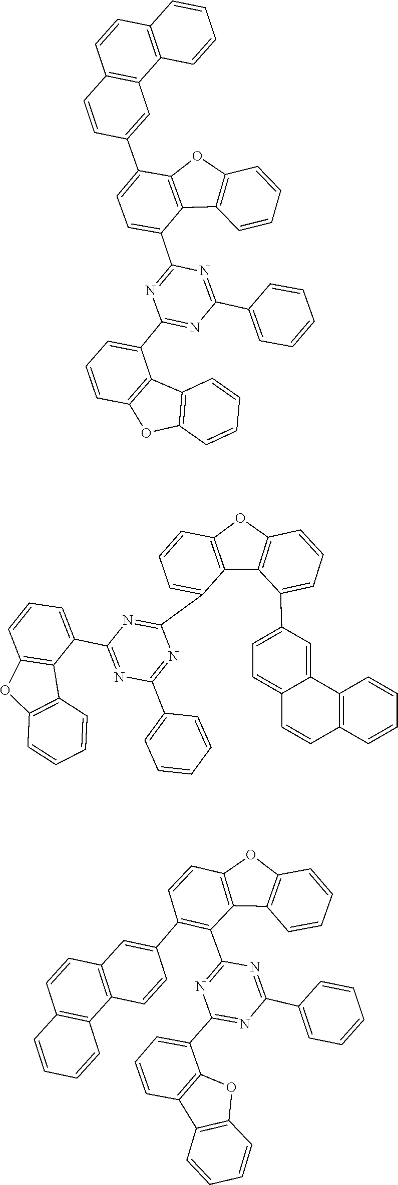 [SMILES: c1ccc(-c2nc(-c3cccc4oc5ccccc5c34)nc(-c3ccc(-c4ccc5ccc6ccccc6c5c4)c4oc5ccccc5c34)n2)cc1 ; c1ccc(-c2nc(-c3cccc4oc5ccccc5c34)nc(-c3cccc4oc5cccc(-c6ccc7ccc8ccccc8c7c6)c5c34)n2)cc1 ; c1ccc(-c2nc(-c3cccc4c3oc3ccccc34)nc(-c3c(-c4ccc5c(ccc6ccccc65)c4)ccc4oc5ccccc5c34)n2)cc1]

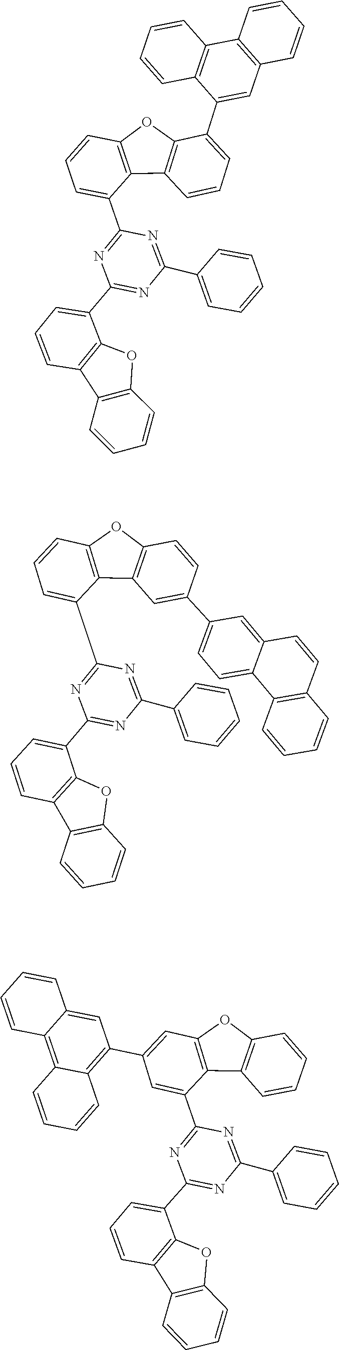 [SMILES: c1ccc(-c2nc(-c3cccc4c3oc3ccccc34)nc(-c3cccc4oc5c(-c6cc7ccccc7c7ccccc67)cccc5c34)n2)cc1 ; c1ccc(-c2nc(-c3cccc4c3oc3ccccc34)nc(-c3cccc4oc5ccc(-c6ccc7c(ccc8ccccc87)c6)cc5c34)n2)cc1 ; c1ccc(-c2nc(-c3cccc4c3oc3ccccc34)nc(-c3cc(-c4cc5ccccc5c5ccccc45)cc4oc5ccccc5c34)n2)cc1]

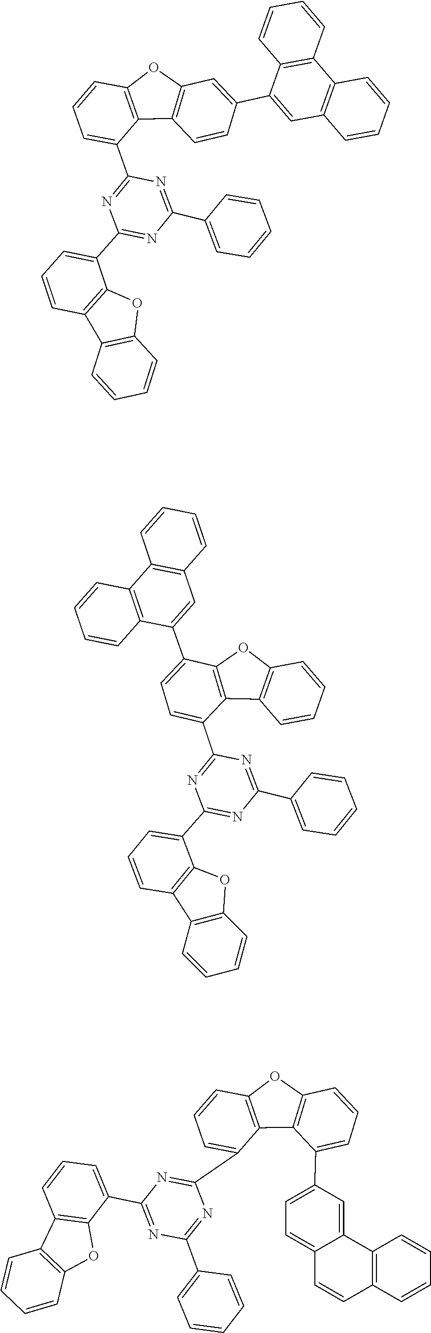 [SMILES: c1ccc(-c2nc(-c3cccc4c3oc3ccccc34)nc(-c3cccc4oc5cc(-c6cc7ccccc7c7ccccc67)ccc5c34)n2)cc1 ; c1ccc(-c2nc(-c3cccc4c3oc3ccccc34)nc(-c3ccc(-c4cc5ccccc5c5ccccc45)c4oc5ccccc5c34)n2)cc1 ; c1ccc(-c2nc(-c3cccc4c3oc3ccccc34)nc(-c3cccc4oc5cccc(-c6ccc7ccc8ccccc8c7c6)c5c34)n2)cc1]

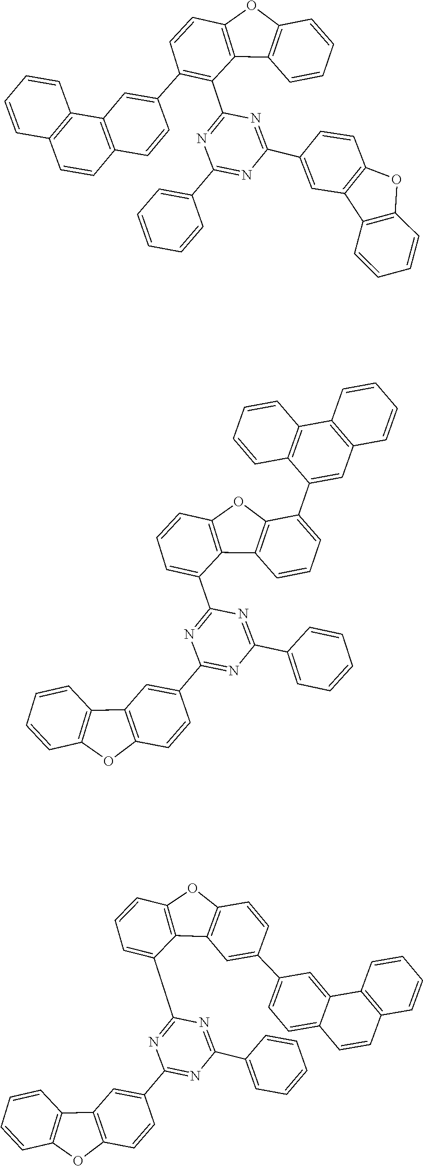 [SMILES: c1ccc(-c2nc(-c3ccc4oc5ccccc5c4c3)nc(-c3c(-c4ccc5ccc6ccccc6c5c4)ccc4oc5ccccc5c34)n2)cc1 ; c1ccc(-c2nc(-c3ccc4oc5ccccc5c4c3)nc(-c3cccc4oc5c(-c6cc7ccccc7c7ccccc67)cccc5c34)n2)cc1 ; c1ccc(-c2nc(-c3ccc4oc5ccccc5c4c3)nc(-c3cccc4oc5ccc(-c6ccc7ccc8ccccc8c7c6)cc5c34)n2)cc1]

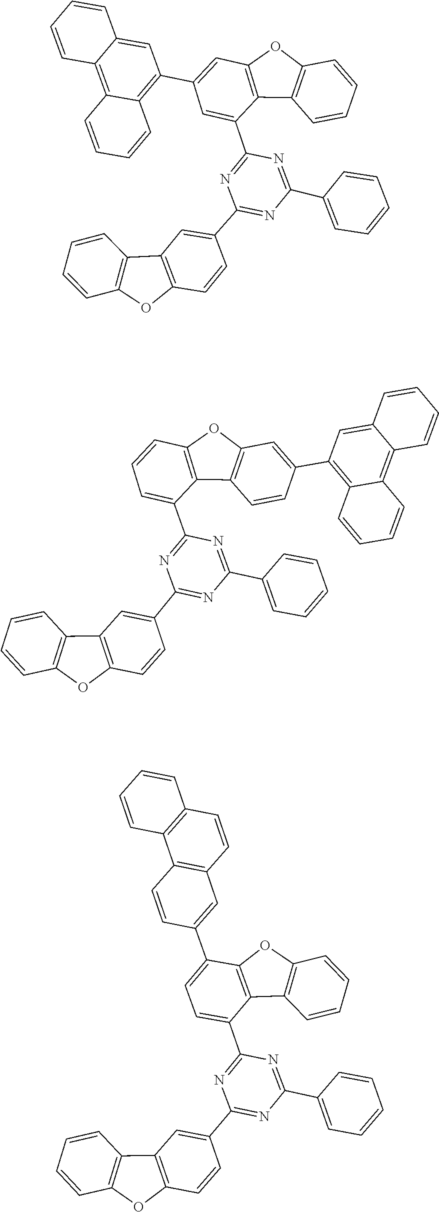 [SMILES: c1ccc(-c2nc(-c3ccc4oc5ccccc5c4c3)nc(-c3cc(-c4cc5ccccc5c5ccccc45)cc4oc5ccccc5c34)n2)cc1 ; c1ccc(-c2nc(-c3ccc4oc5ccccc5c4c3)nc(-c3cccc4oc5cc(-c6cc7ccccc7c7ccccc67)ccc5c34)n2)cc1 ; c1ccc(-c2nc(-c3ccc4oc5ccccc5c4c3)nc(-c3ccc(-c4ccc5c(ccc6ccccc65)c4)c4oc5ccccc5c34)n2)cc1]

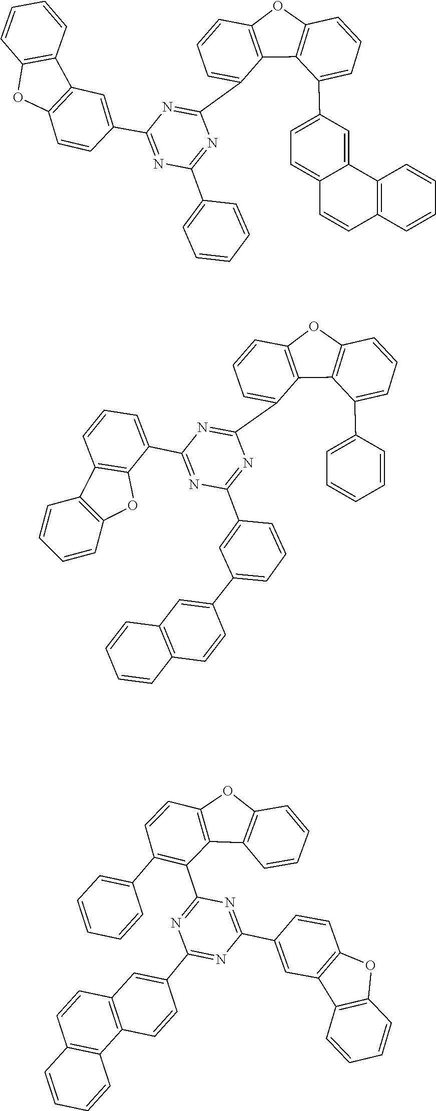 [SMILES: c1ccc(-c2nc(-c3ccc4oc5ccccc5c4c3)nc(-c3cccc4oc5cccc(-c6ccc7ccc8ccccc8c7c6)c5c34)n2)cc1 ; c1ccc(-c2cccc3oc4cccc(-c5nc(-c6cccc(-c7ccc8ccccc8c7)c6)nc(-c6cccc7c6oc6ccccc67)n5)c4c23)cc1 ; c1ccc(-c2ccc3oc4ccccc4c3c2-c2nc(-c3ccc4c(ccc5ccccc54)c3)nc(-c3ccc4oc5ccccc5c4c3)n2)cc1]

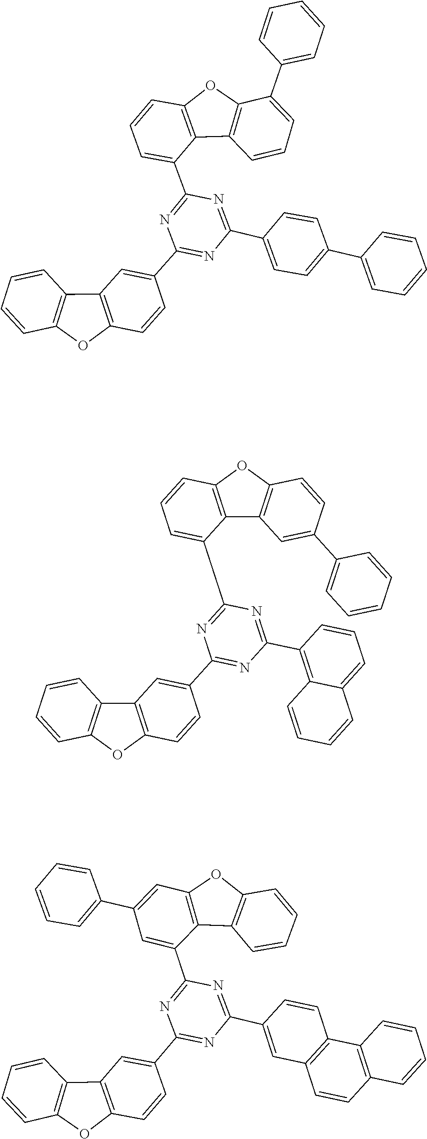 [SMILES: c1ccc(-c2ccc(-c3nc(-c4ccc5oc6ccccc6c5c4)nc(-c4cccc5oc6c(-c7ccccc7)cccc6c45)n3)cc2)cc1 ; c1ccc(-c2ccc3oc4cccc(-c5nc(-c6ccc7oc8ccccc8c7c6)nc(-c6cccc7ccccc67)n5)c4c3c2)cc1 ; c1ccc(-c2cc(-c3nc(-c4ccc5c(ccc6ccccc65)c4)nc(-c4ccc5oc6ccccc6c5c4)n3)c3c(c2)oc2ccccc23)cc1]

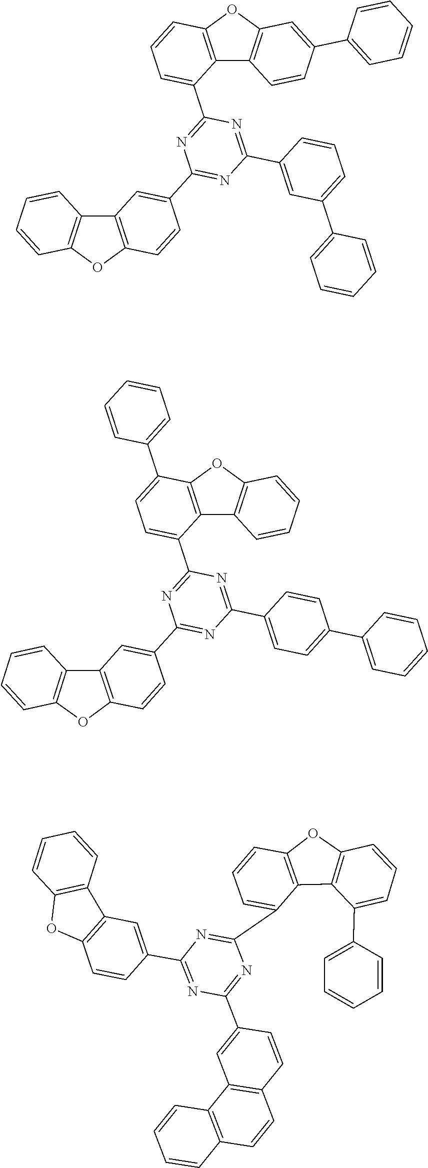 [SMILES: c1ccc(-c2cccc(-c3nc(-c4ccc5oc6ccccc6c5c4)nc(-c4cccc5oc6cc(-c7ccccc7)ccc6c45)n3)c2)cc1 ; c1ccc(-c2ccc(-c3nc(-c4ccc5oc6ccccc6c5c4)nc(-c4ccc(-c5ccccc5)c5oc6ccccc6c45)n3)cc2)cc1 ; c1ccc(-c2cccc3oc4cccc(-c5nc(-c6ccc7ccc8ccccc8c7c6)nc(-c6ccc7oc8ccccc8c7c6)n5)c4c23)cc1]

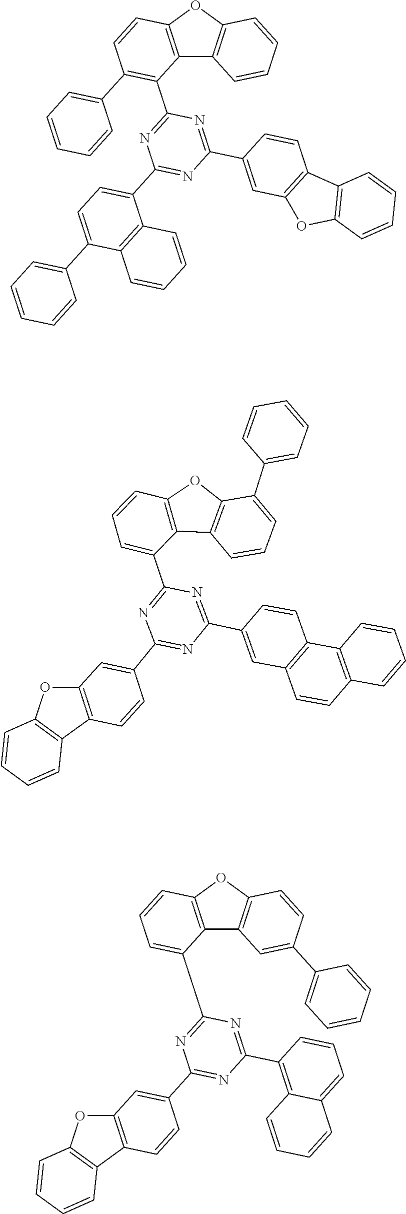 [SMILES: c1ccc(-c2ccc3oc4ccccc4c3c2-c2nc(-c3ccc4c(c3)oc3ccccc34)nc(-c3ccc(-c4ccccc4)c4ccccc34)n2)cc1 ; c1ccc(-c2cccc3c2oc2cccc(-c4nc(-c5ccc6c(ccc7ccccc76)c5)nc(-c5ccc6c(c5)oc5ccccc56)n4)c23)cc1 ; c1ccc(-c2ccc3oc4cccc(-c5nc(-c6ccc7c(c6)oc6ccccc67)nc(-c6cccc7ccccc67)n5)c4c3c2)cc1]

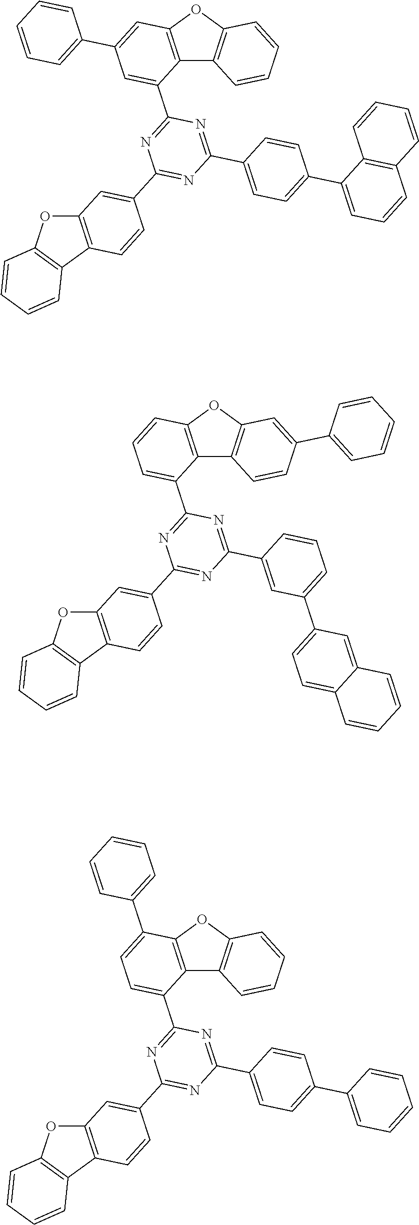 [SMILES: c1ccc(-c2cc(-c3nc(-c4ccc(-c5cccc6ccccc56)cc4)nc(-c4ccc5c(c4)oc4ccccc45)n3)c3c(c2)oc2ccccc23)cc1 ; c1ccc(-c2ccc3c(c2)oc2cccc(-c4nc(-c5cccc(-c6ccc7ccccc7c6)c5)nc(-c5ccc6c(c5)oc5ccccc56)n4)c23)cc1 ; c1ccc(-c2ccc(-c3nc(-c4ccc5c(c4)oc4ccccc45)nc(-c4ccc(-c5ccccc5)c5oc6ccccc6c45)n3)cc2)cc1]

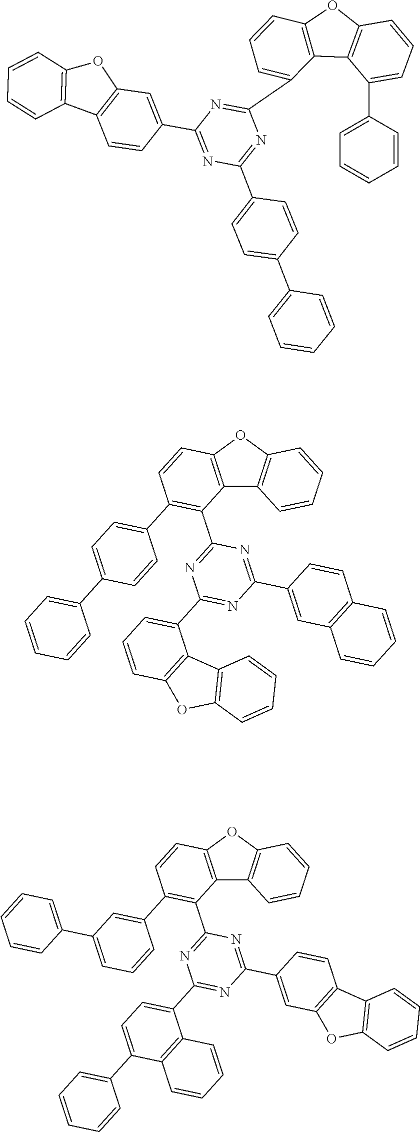 [SMILES: c1ccc(-c2ccc(-c3nc(-c4ccc5c(c4)oc4ccccc45)nc(-c4cccc5oc6cccc(-c7ccccc7)c6c45)n3)cc2)cc1 ; c1ccc(-c2ccc(-c3ccc4oc5ccccc5c4c3-c3nc(-c4ccc5ccccc5c4)nc(-c4cccc5oc6ccccc6c45)n3)cc2)cc1 ; c1ccc(-c2cccc(-c3ccc4oc5ccccc5c4c3-c3nc(-c4ccc5c(c4)oc4ccccc45)nc(-c4ccc(-c5ccccc5)c5ccccc45)n3)c2)cc1]

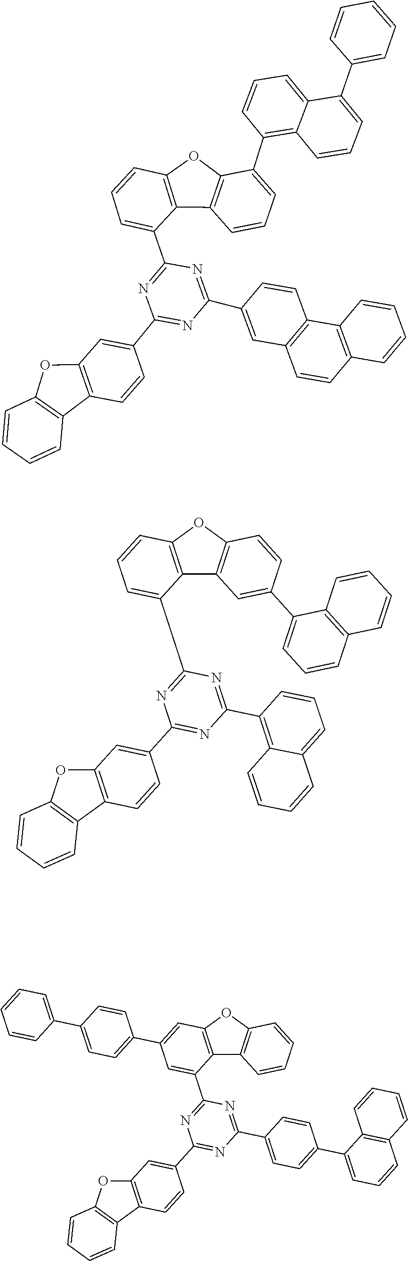 [SMILES: c1ccc(-c2cccc3c(-c4cccc5c4oc4cccc(-c6nc(-c7ccc8c(ccc9ccccc98)c7)nc(-c7ccc8c(c7)oc7ccccc78)n6)c45)cccc23)cc1 ; c1ccc2c(-c3ccc4oc5cccc(-c6nc(-c7ccc8c(c7)oc7ccccc78)nc(-c7cccc8ccccc78)n6)c5c4c3)cccc2c1 ; c1ccc(-c2ccc(-c3cc(-c4nc(-c5ccc(-c6cccc7ccccc67)cc5)nc(-c5ccc6c(c5)oc5ccccc56)n4)c4c(c3)oc3ccccc34)cc2)cc1]

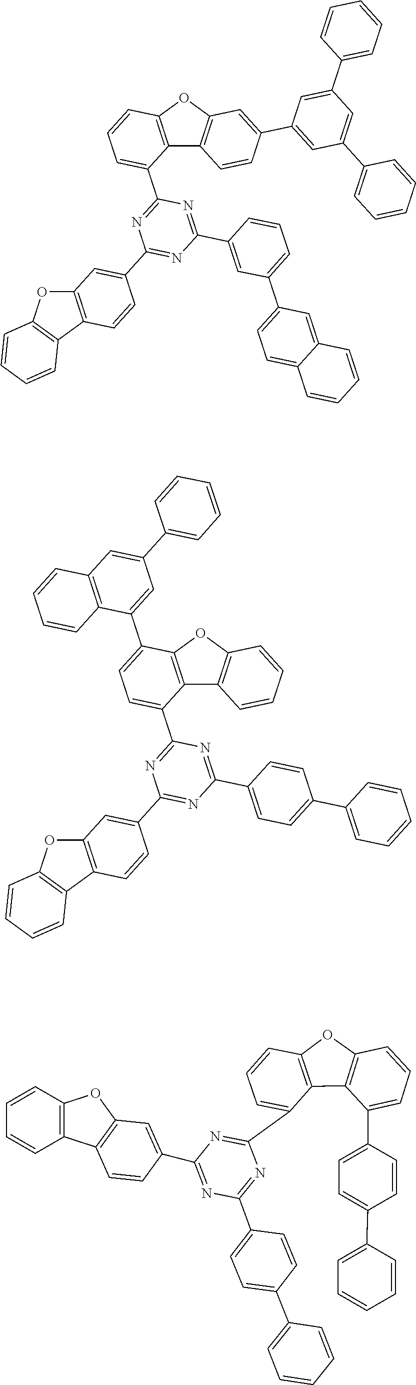 [SMILES: c1ccc(-c2cc(-c3ccccc3)cc(-c3ccc4c(c3)oc3cccc(-c5nc(-c6cccc(-c7ccc8ccccc8c7)c6)nc(-c6ccc7c(c6)oc6ccccc67)n5)c34)c2)cc1 ; c1ccc(-c2ccc(-c3nc(-c4ccc5c(c4)oc4ccccc45)nc(-c4ccc(-c5cc(-c6ccccc6)cc6ccccc56)c5oc6ccccc6c45)n3)cc2)cc1 ; c1ccc(-c2ccc(-c3nc(-c4ccc5c(c4)oc4ccccc45)nc(-c4cccc5oc6cccc(-c7ccc(-c8ccccc8)cc7)c6c45)n3)cc2)cc1]

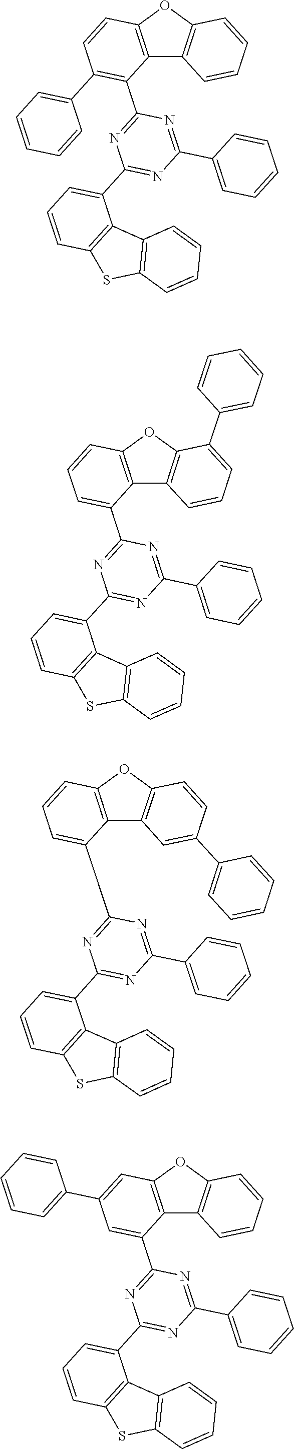 [SMILES: c1ccc(-c2nc(-c3c(-c4ccccc4)ccc4oc5ccccc5c34)nc(-c3cccc4sc5ccccc5c34)n2)cc1 ; c1ccc(-c2nc(-c3cccc4oc5c(-c6ccccc6)cccc5c34)nc(-c3cccc4sc5ccccc5c34)n2)cc1 ; c1ccc(-c2ccc3oc4cccc(-c5nc(-c6ccccc6)nc(-c6cccc7sc8ccccc8c67)n5)c4c3c2)cc1 ; c1ccc(-c2cc(-c3nc(-c4ccccc4)nc(-c4cccc5sc6ccccc6c45)n3)c3c(c2)oc2ccccc23)cc1]

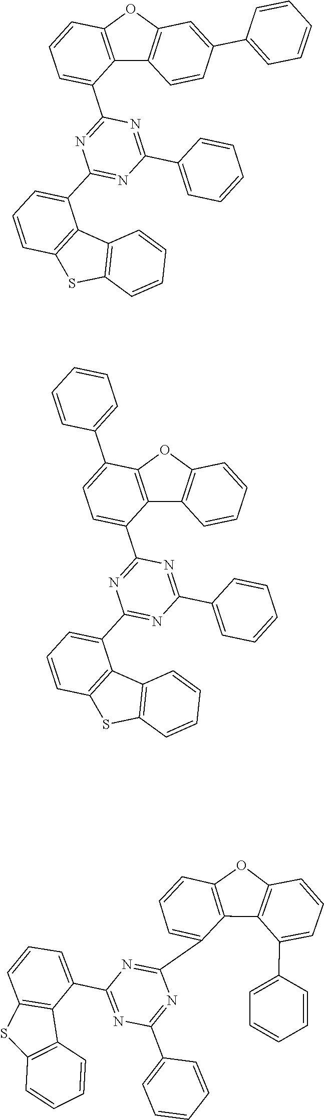 [SMILES: c1ccc(-c2ccc3c(c2)oc2cccc(-c4nc(-c5ccccc5)nc(-c5cccc6sc7ccccc7c56)n4)c23)cc1 ; c1ccc(-c2nc(-c3cccc4sc5ccccc5c34)nc(-c3ccc(-c4ccccc4)c4oc5ccccc5c34)n2)cc1 ; c1ccc(-c2nc(-c3cccc4sc5ccccc5c34)nc(-c3cccc4oc5cccc(-c6ccccc6)c5c34)n2)cc1]

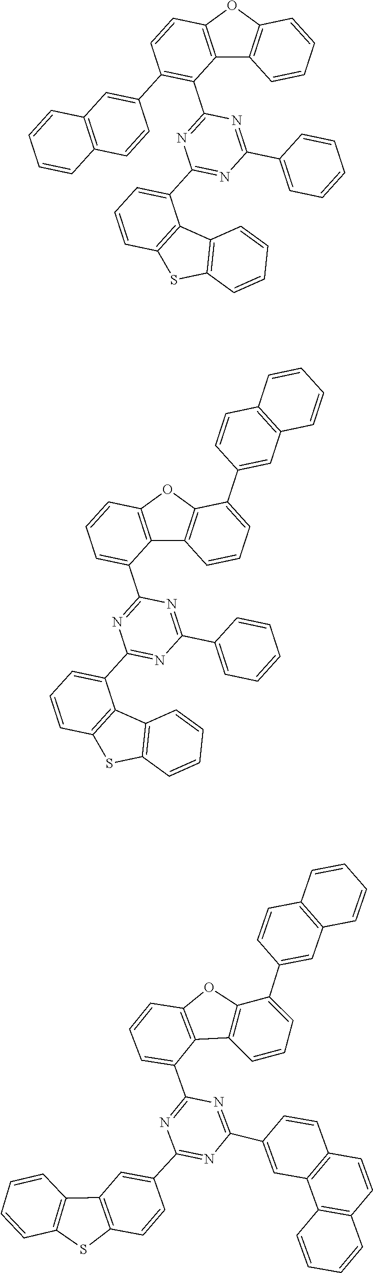 [SMILES: c1ccc(-c2nc(-c3c(-c4ccc5ccccc5c4)ccc4oc5ccccc5c34)nc(-c3cccc4sc5ccccc5c34)n2)cc1 ; c1ccc(-c2nc(-c3cccc4oc5c(-c6ccc7ccccc7c6)cccc5c34)nc(-c3cccc4sc5ccccc5c34)n2)cc1 ; c1ccc2cc(-c3cccc4c3oc3cccc(-c5nc(-c6ccc7ccc8ccccc8c7c6)nc(-c6ccc7sc8ccccc8c7c6)n5)c34)ccc2c1]

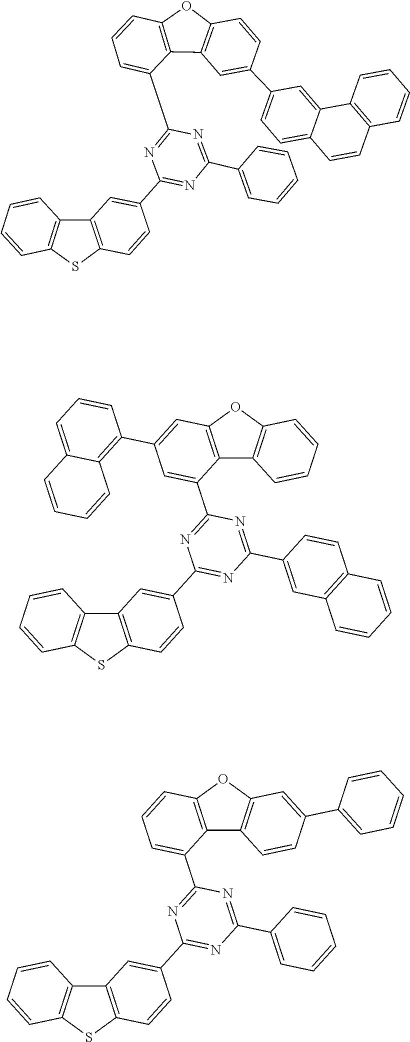 [SMILES: c1ccc(-c2nc(-c3ccc4sc5ccccc5c4c3)nc(-c3cccc4oc5ccc(-c6ccc7ccc8ccccc8c7c6)cc5c34)n2)cc1 ; c1ccc2cc(-c3nc(-c4ccc5sc6ccccc6c5c4)nc(-c4cc(-c5cccc6ccccc56)cc5oc6ccccc6c45)n3)ccc2c1 ; c1ccc(-c2ccc3c(c2)oc2cccc(-c4nc(-c5ccccc5)nc(-c5ccc6sc7ccccc7c6c5)n4)c23)cc1]

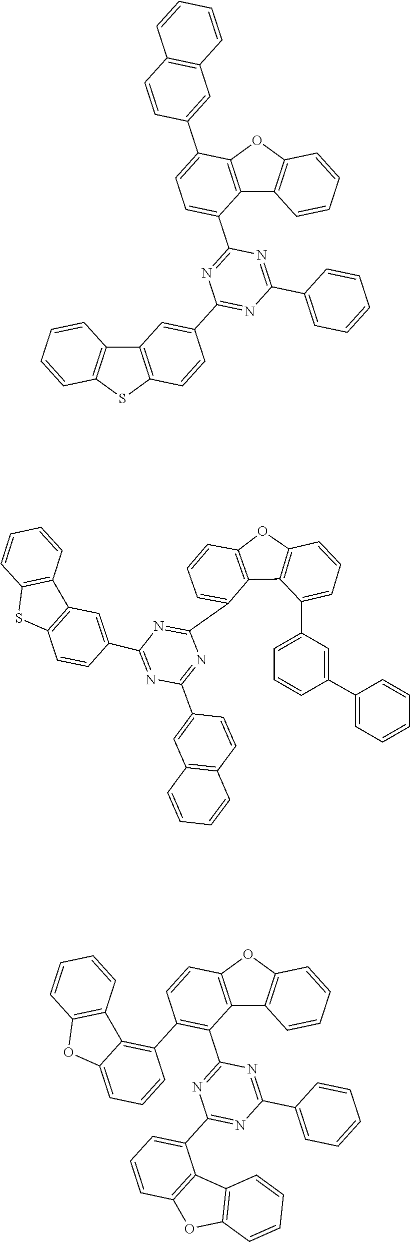 [SMILES: c1ccc(-c2nc(-c3ccc4sc5ccccc5c4c3)nc(-c3ccc(-c4ccc5ccccc5c4)c4oc5ccccc5c34)n2)cc1 ; c1ccc(-c2cccc(-c3cccc4oc5cccc(-c6nc(-c7ccc8ccccc8c7)nc(-c7ccc8sc9ccccc9c8c7)n6)c5c34)c2)cc1 ; c1ccc(-c2nc(-c3cccc4oc5ccccc5c34)nc(-c3c(-c4cccc5oc6ccccc6c45)ccc4oc5ccccc5c34)n2)cc1]

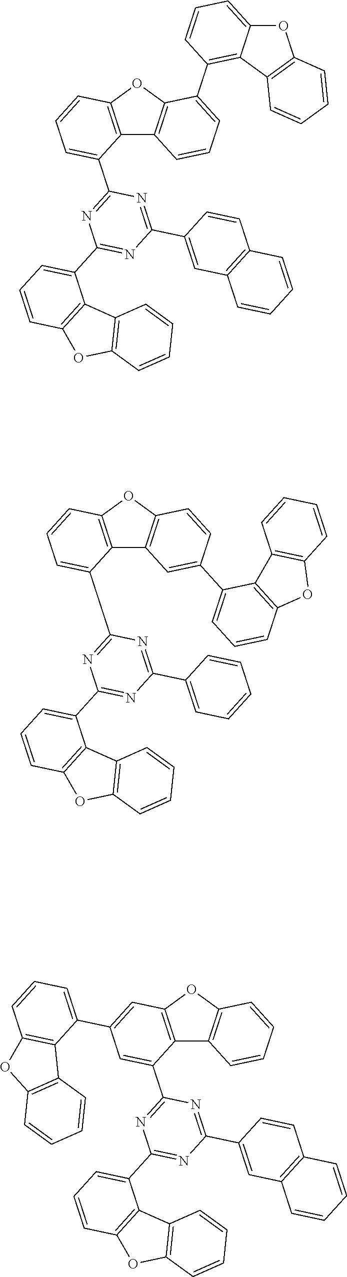 [SMILES: c1ccc2cc(-c3nc(-c4cccc5oc6ccccc6c45)nc(-c4cccc5oc6c(-c7cccc8oc9ccccc9c78)cccc6c45)n3)ccc2c1 ; c1ccc(-c2nc(-c3cccc4oc5ccccc5c34)nc(-c3cccc4oc5ccc(-c6cccc7oc8ccccc8c67)cc5c34)n2)cc1 ; c1ccc2cc(-c3nc(-c4cccc5oc6ccccc6c45)nc(-c4cc(-c5cccc6oc7ccccc7c56)cc5oc6ccccc6c45)n3)ccc2c1]

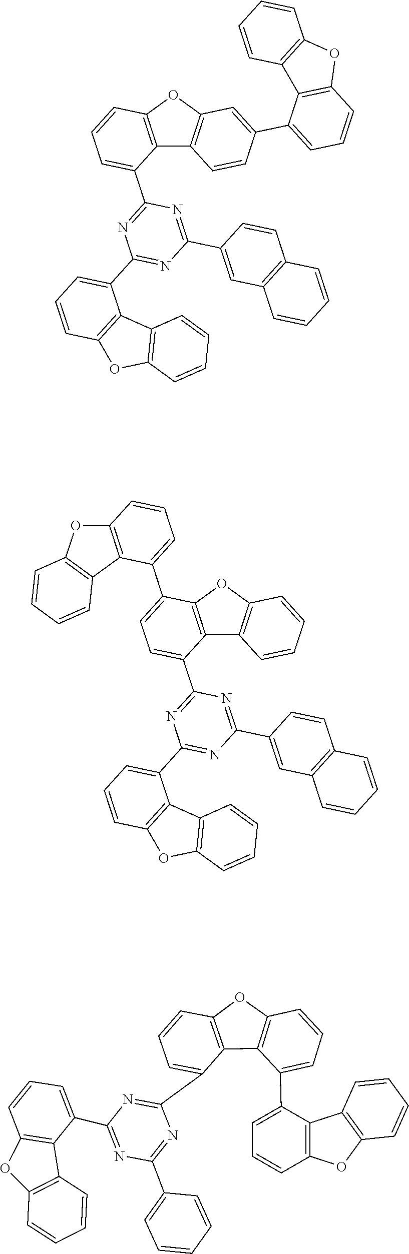 [SMILES: c1ccc2cc(-c3nc(-c4cccc5oc6ccccc6c45)nc(-c4cccc5oc6cc(-c7cccc8oc9ccccc9c78)ccc6c45)n3)ccc2c1 ; c1ccc2cc(-c3nc(-c4cccc5oc6ccccc6c45)nc(-c4ccc(-c5cccc6oc7ccccc7c56)c5oc6ccccc6c45)n3)ccc2c1 ; c1ccc(-c2nc(-c3cccc4oc5ccccc5c34)nc(-c3cccc4oc5cccc(-c6cccc7oc8ccccc8c67)c5c34)n2)cc1]

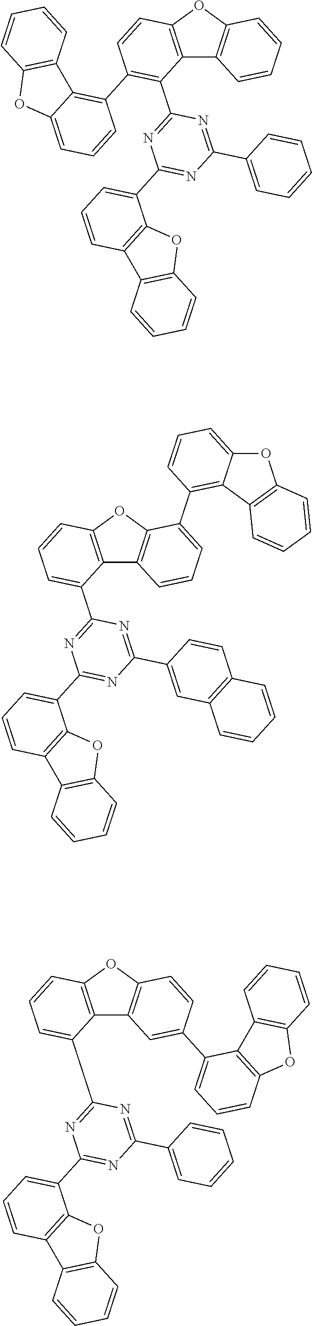 [SMILES: c1ccc(-c2nc(-c3cccc4c3oc3ccccc34)nc(-c3c(-c4cccc5oc6ccccc6c45)ccc4oc5ccccc5c34)n2)cc1 ; c1ccc2cc(-c3nc(-c4cccc5c4oc4ccccc45)nc(-c4cccc5oc6c(-c7cccc8oc9ccccc9c78)cccc6c45)n3)ccc2c1 ; c1ccc(-c2nc(-c3cccc4c3oc3ccccc34)nc(-c3cccc4oc5ccc(-c6cccc7oc8ccccc8c67)cc5c34)n2)cc1]

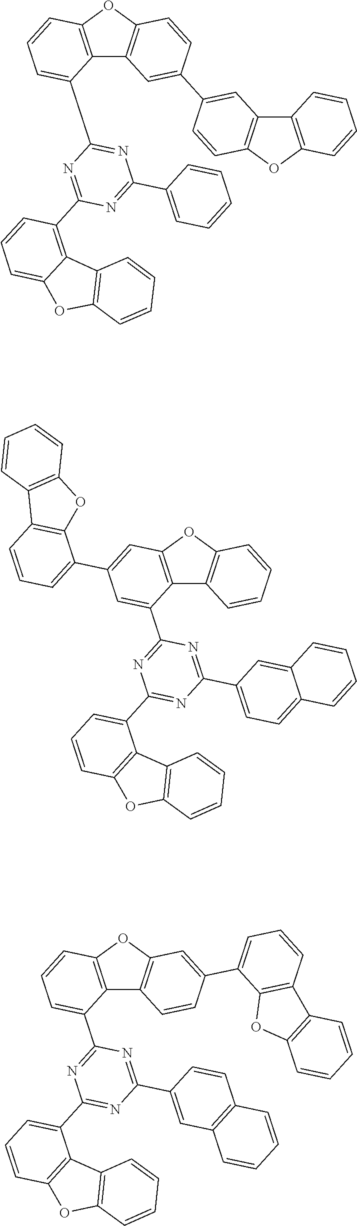 [SMILES: c1ccc(-c2nc(-c3cccc4oc5ccccc5c34)nc(-c3cccc4oc5ccc(-c6ccc7oc8ccccc8c7c6)cc5c34)n2)cc1 ; c1ccc2cc(-c3nc(-c4cccc5oc6ccccc6c45)nc(-c4cc(-c5cccc6c5oc5ccccc56)cc5oc6ccccc6c45)n3)ccc2c1 ; c1ccc2cc(-c3nc(-c4cccc5oc6ccccc6c45)nc(-c4cccc5oc6cc(-c7cccc8c7oc7ccccc78)ccc6c45)n3)ccc2c1]

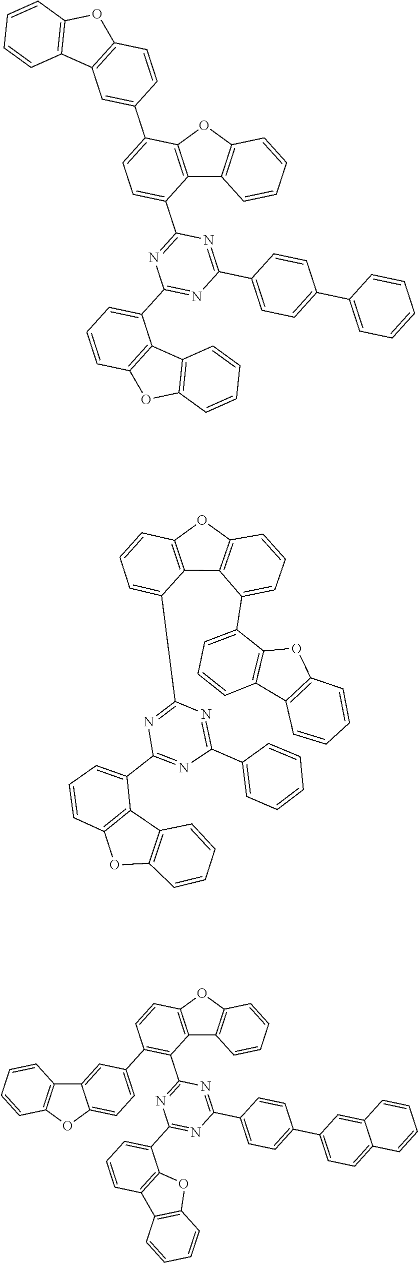 [SMILES: c1ccc(-c2ccc(-c3nc(-c4cccc5oc6ccccc6c45)nc(-c4ccc(-c5ccc6oc7ccccc7c6c5)c5oc6ccccc6c45)n3)cc2)cc1 ; c1ccc(-c2nc(-c3cccc4oc5ccccc5c34)nc(-c3cccc4oc5cccc(-c6cccc7c6oc6ccccc67)c5c34)n2)cc1 ; c1ccc2cc(-c3ccc(-c4nc(-c5cccc6c5oc5ccccc56)nc(-c5c(-c6ccc7oc8ccccc8c7c6)ccc6oc7ccccc7c56)n4)cc3)ccc2c1]

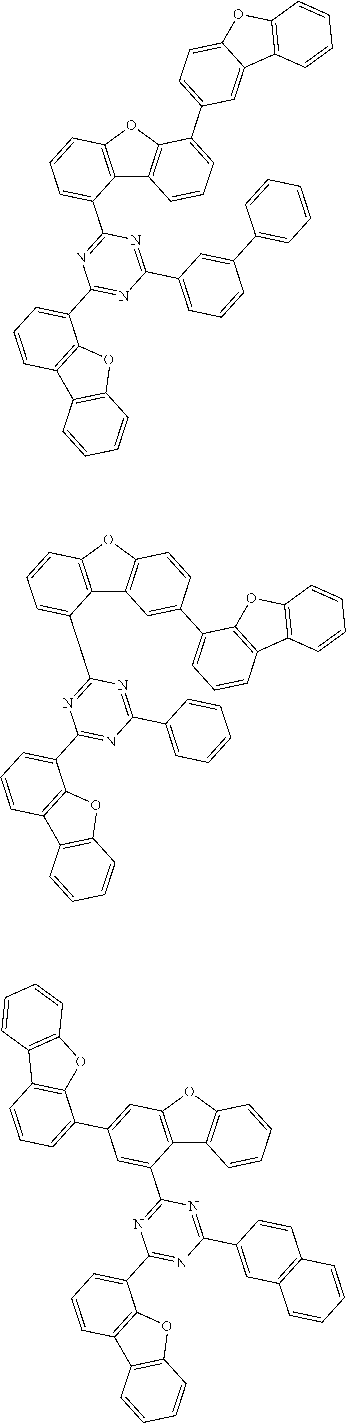 [SMILES: c1ccc(-c2cccc(-c3nc(-c4cccc5c4oc4ccccc45)nc(-c4cccc5oc6c(-c7ccc8oc9ccccc9c8c7)cccc6c45)n3)c2)cc1 ; c1ccc(-c2nc(-c3cccc4c3oc3ccccc34)nc(-c3cccc4oc5ccc(-c6cccc7c6oc6ccccc67)cc5c34)n2)cc1 ; c1ccc2cc(-c3nc(-c4cccc5c4oc4ccccc45)nc(-c4cc(-c5cccc6c5oc5ccccc56)cc5oc6ccccc6c45)n3)ccc2c1]

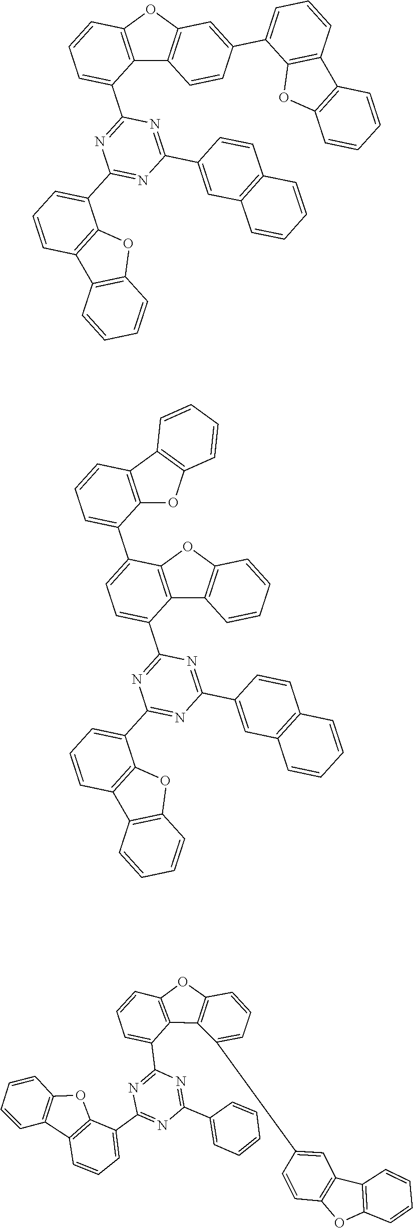 [SMILES: c1ccc2cc(-c3nc(-c4cccc5c4oc4ccccc45)nc(-c4cccc5oc6cc(-c7cccc8c7oc7ccccc78)ccc6c45)n3)ccc2c1 ; c1ccc2cc(-c3nc(-c4cccc5c4oc4ccccc45)nc(-c4ccc(-c5cccc6c5oc5ccccc56)c5oc6ccccc6c45)n3)ccc2c1 ; c1ccc(-c2nc(-c3cccc4c3oc3ccccc34)nc(-c3cccc4oc5cccc(-c6ccc7oc8ccccc8c7c6)c5c34)n2)cc1]

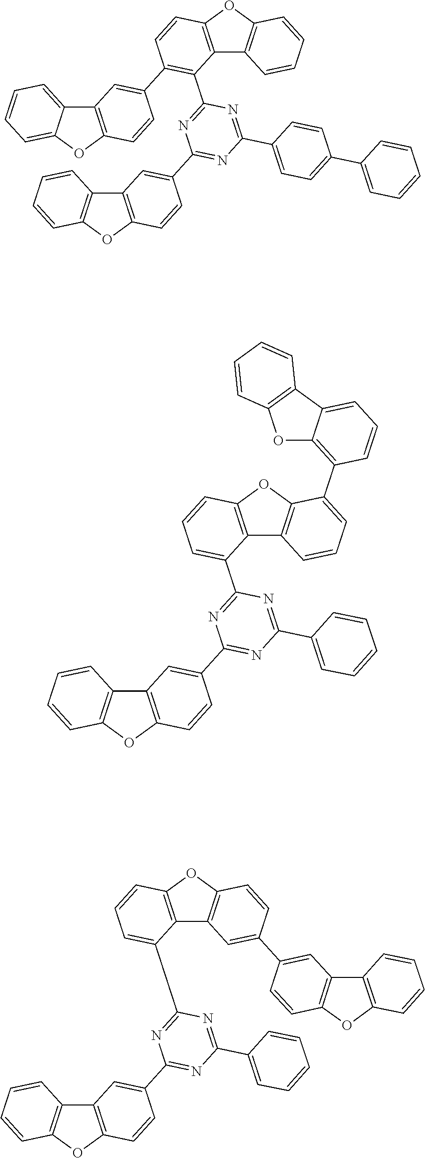 [SMILES: c1ccc(-c2ccc(-c3nc(-c4ccc5oc6ccccc6c5c4)nc(-c4c(-c5ccc6oc7ccccc7c6c5)ccc5oc6ccccc6c45)n3)cc2)cc1 ; c1ccc(-c2nc(-c3ccc4oc5ccccc5c4c3)nc(-c3cccc4oc5c(-c6cccc7c6oc6ccccc67)cccc5c34)n2)cc1 ; c1ccc(-c2nc(-c3ccc4oc5ccccc5c4c3)nc(-c3cccc4oc5ccc(-c6ccc7oc8ccccc8c7c6)cc5c34)n2)cc1]

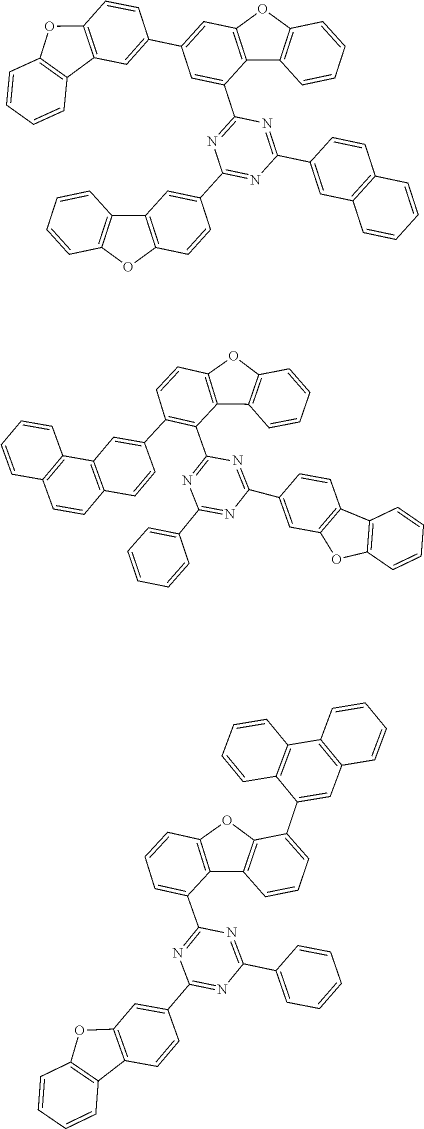 [SMILES: c1ccc2cc(-c3nc(-c4ccc5oc6ccccc6c5c4)nc(-c4cc(-c5ccc6oc7ccccc7c6c5)cc5oc6ccccc6c45)n3)ccc2c1 ; c1ccc(-c2nc(-c3ccc4c(c3)oc3ccccc34)nc(-c3c(-c4ccc5ccc6ccccc6c5c4)ccc4oc5ccccc5c34)n2)cc1 ; c1ccc(-c2nc(-c3ccc4c(c3)oc3ccccc34)nc(-c3cccc4oc5c(-c6cc7ccccc7c7ccccc67)cccc5c34)n2)cc1]

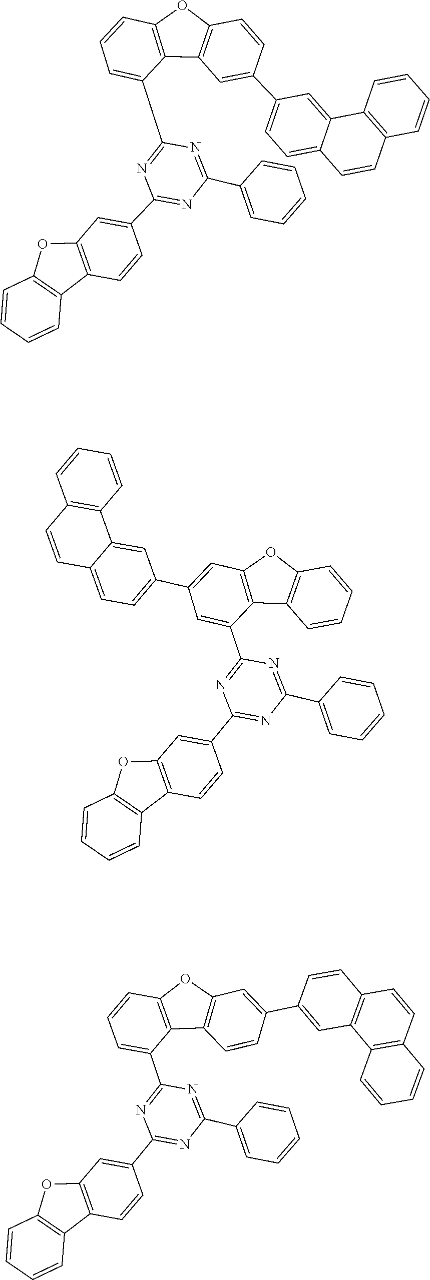 [SMILES: c1ccc(-c2nc(-c3ccc4c(c3)oc3ccccc34)nc(-c3cccc4oc5ccc(-c6ccc7ccc8ccccc8c7c6)cc5c34)n2)cc1 ; c1ccc(-c2nc(-c3ccc4c(c3)oc3ccccc34)nc(-c3cc(-c4ccc5ccc6ccccc6c5c4)cc4oc5ccccc5c34)n2)cc1 ; c1ccc(-c2nc(-c3ccc4c(c3)oc3ccccc34)nc(-c3cccc4oc5cc(-c6ccc7ccc8ccccc8c7c6)ccc5c34)n2)cc1]

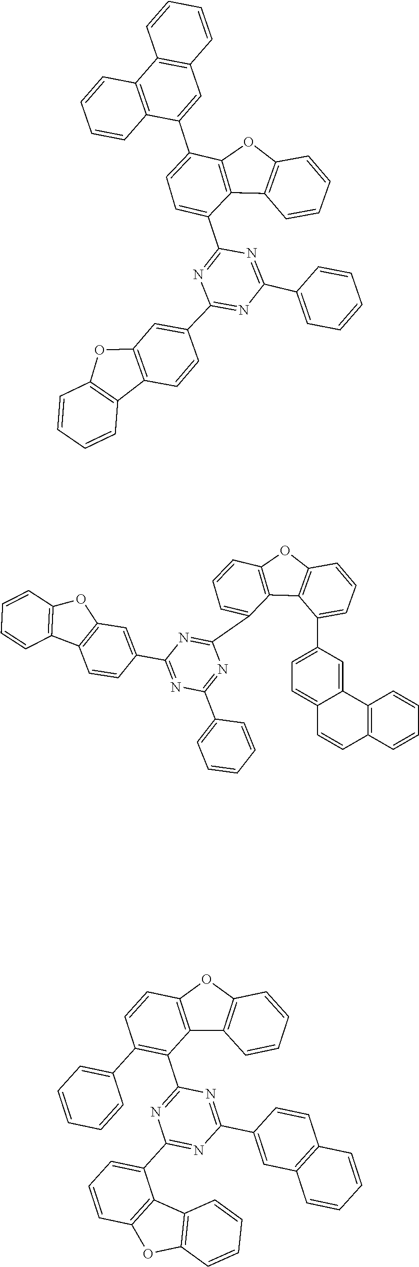 [SMILES: c1ccc(-c2nc(-c3ccc4c(c3)oc3ccccc34)nc(-c3ccc(-c4cc5ccccc5c5ccccc45)c4oc5ccccc5c34)n2)cc1 ; c1ccc(-c2nc(-c3ccc4c(c3)oc3ccccc34)nc(-c3cccc4oc5cccc(-c6ccc7ccc8ccccc8c7c6)c5c34)n2)cc1 ; c1ccc(-c2ccc3oc4ccccc4c3c2-c2nc(-c3ccc4ccccc4c3)nc(-c3cccc4oc5ccccc5c34)n2)cc1]

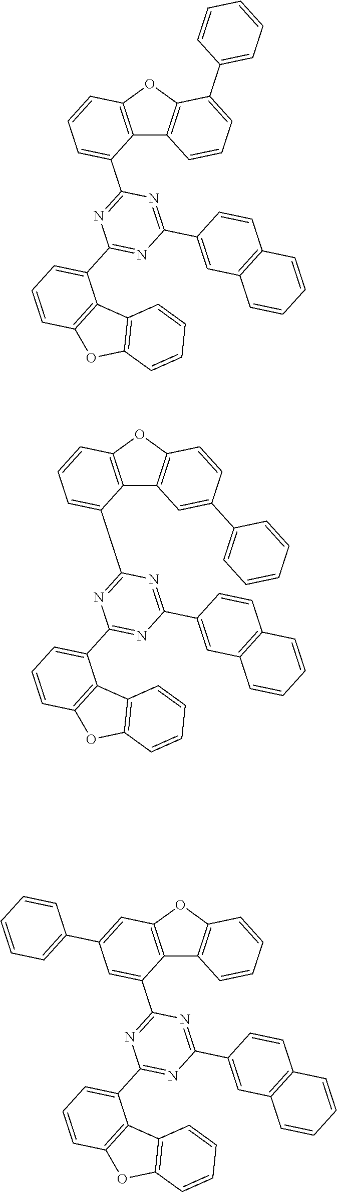 [SMILES: c1ccc(-c2cccc3c2oc2cccc(-c4nc(-c5ccc6ccccc6c5)nc(-c5cccc6oc7ccccc7c56)n4)c23)cc1 ; c1ccc(-c2ccc3oc4cccc(-c5nc(-c6ccc7ccccc7c6)nc(-c6cccc7oc8ccccc8c67)n5)c4c3c2)cc1 ; c1ccc(-c2cc(-c3nc(-c4ccc5ccccc5c4)nc(-c4cccc5oc6ccccc6c45)n3)c3c(c2)oc2ccccc23)cc1]

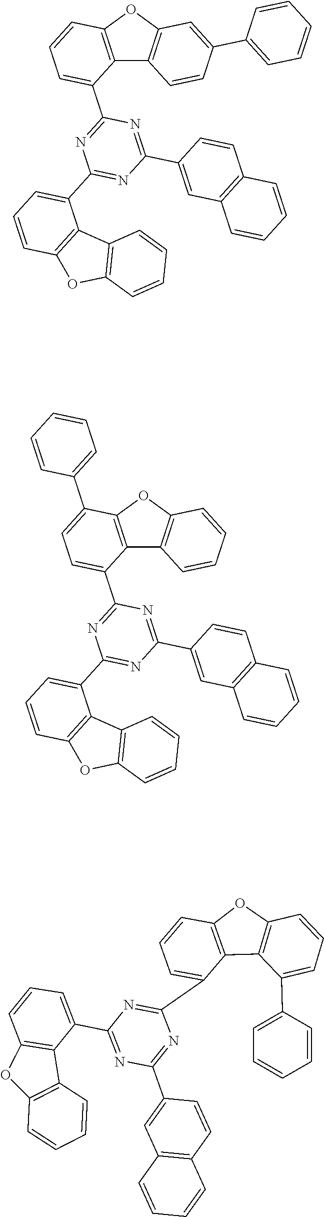 [SMILES: c1ccc(-c2ccc3c(c2)oc2cccc(-c4nc(-c5ccc6ccccc6c5)nc(-c5cccc6oc7ccccc7c56)n4)c23)cc1 ; c1ccc(-c2ccc(-c3nc(-c4ccc5ccccc5c4)nc(-c4cccc5oc6ccccc6c45)n3)c3c2oc2ccccc23)cc1 ; c1ccc(-c2cccc3oc4cccc(-c5nc(-c6ccc7ccccc7c6)nc(-c6cccc7oc8ccccc8c67)n5)c4c23)cc1]

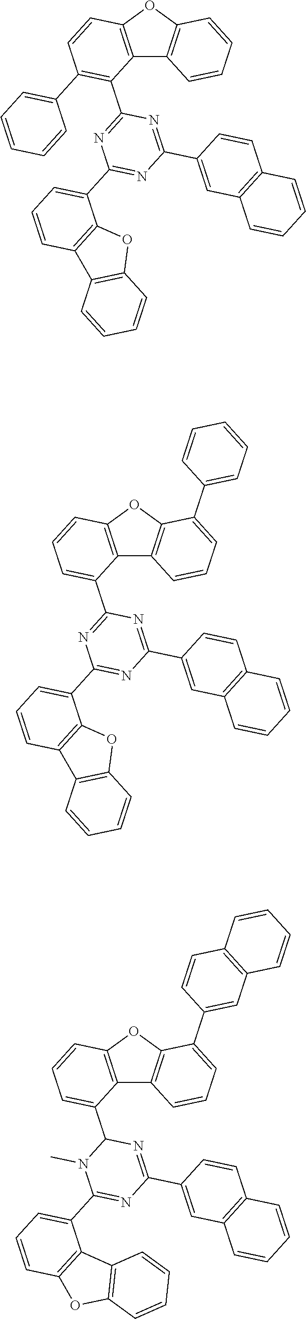 [SMILES: c1ccc(-c2ccc3oc4ccccc4c3c2-c2nc(-c3ccc4ccccc4c3)nc(-c3cccc4c3oc3ccccc34)n2)cc1 ; c1ccc(-c2cccc3c2oc2cccc(-c4nc(-c5ccc6ccccc6c5)nc(-c5cccc6c5oc5ccccc56)n4)c23)cc1 ; CN1C(c2cccc3oc4ccccc4c23)=NC(c2ccc3ccccc3c2)=NC1c1cccc2oc3c(-c4ccc5ccccc5c4)cccc3c12]

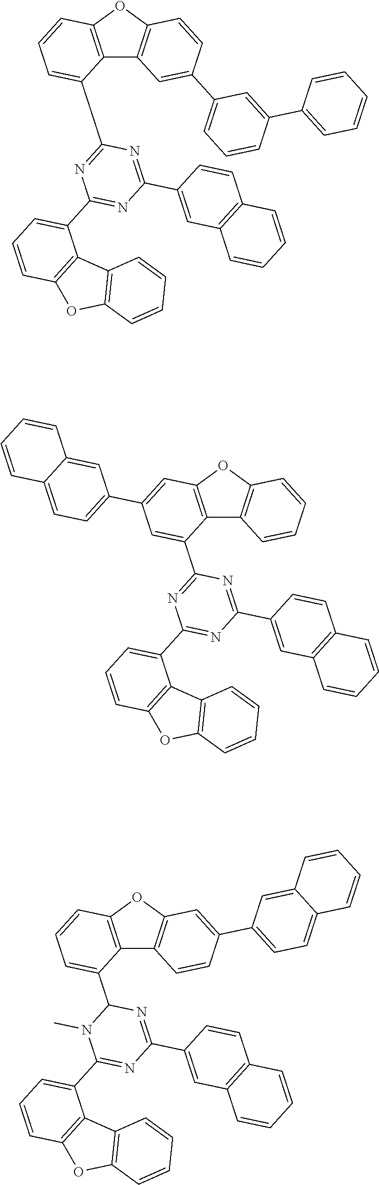 [SMILES: c1ccc(-c2cccc(-c3ccc4oc5cccc(-c6nc(-c7ccc8ccccc8c7)nc(-c7cccc8oc9ccccc9c78)n6)c5c4c3)c2)cc1 ; c1ccc2cc(-c3cc(-c4nc(-c5ccc6ccccc6c5)nc(-c5cccc6oc7ccccc7c56)n4)c4c(c3)oc3ccccc34)ccc2c1 ; CN1C(c2cccc3oc4ccccc4c23)=NC(c2ccc3ccccc3c2)=NC1c1cccc2oc3cc(-c4ccc5ccccc5c4)ccc3c12]

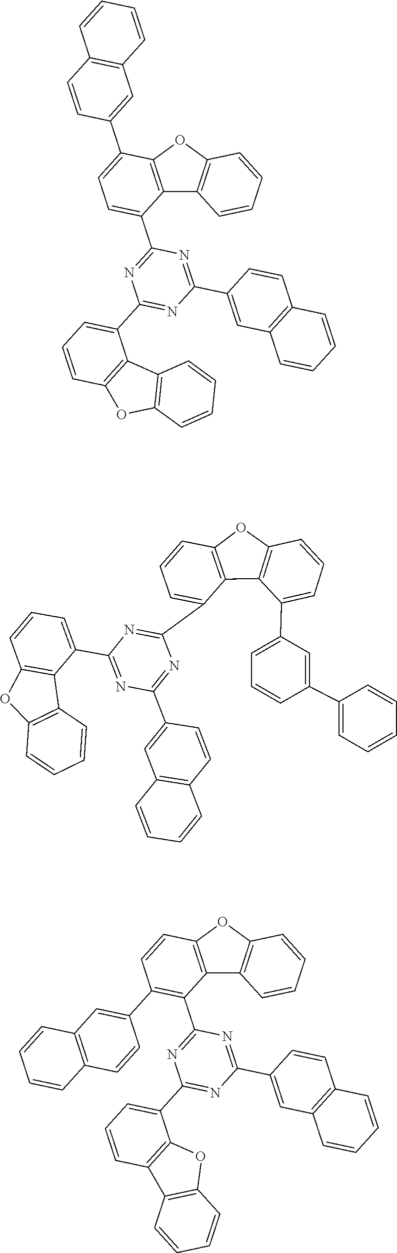 [SMILES: c1ccc2cc(-c3nc(-c4cccc5oc6ccccc6c45)nc(-c4ccc(-c5ccc6ccccc6c5)c5oc6ccccc6c45)n3)ccc2c1 ; c1ccc(-c2cccc(-c3cccc4oc5cccc(-c6nc(-c7ccc8ccccc8c7)nc(-c7cccc8oc9ccccc9c78)n6)c5c34)c2)cc1 ; c1ccc2cc(-c3nc(-c4cccc5c4oc4ccccc45)nc(-c4c(-c5ccc6ccccc6c5)ccc5oc6ccccc6c45)n3)ccc2c1]

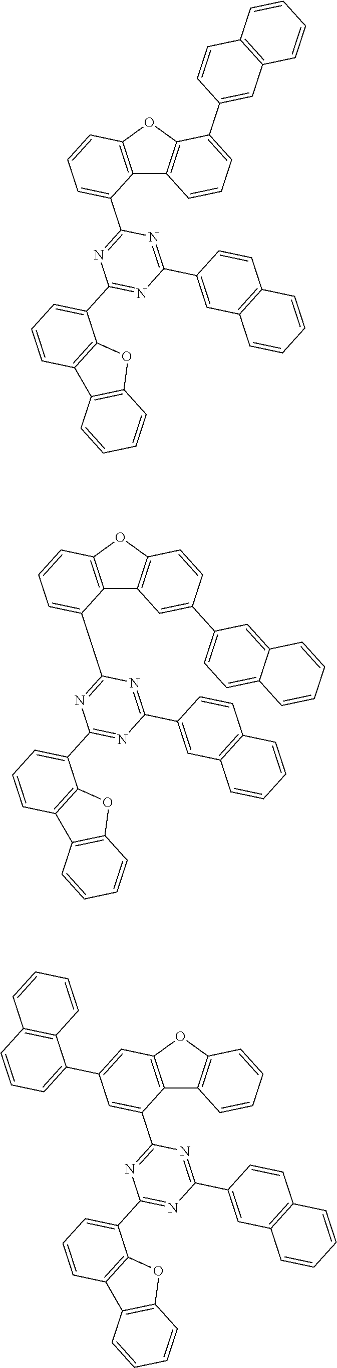 [SMILES: c1ccc2cc(-c3nc(-c4cccc5c4oc4ccccc45)nc(-c4cccc5oc6c(-c7ccc8ccccc8c7)cccc6c45)n3)ccc2c1 ; c1ccc2cc(-c3ccc4oc5cccc(-c6nc(-c7ccc8ccccc8c7)nc(-c7cccc8c7oc7ccccc78)n6)c5c4c3)ccc2c1 ; c1ccc2cc(-c3nc(-c4cccc5c4oc4ccccc45)nc(-c4cc(-c5cccc6ccccc56)cc5oc6ccccc6c45)n3)ccc2c1]

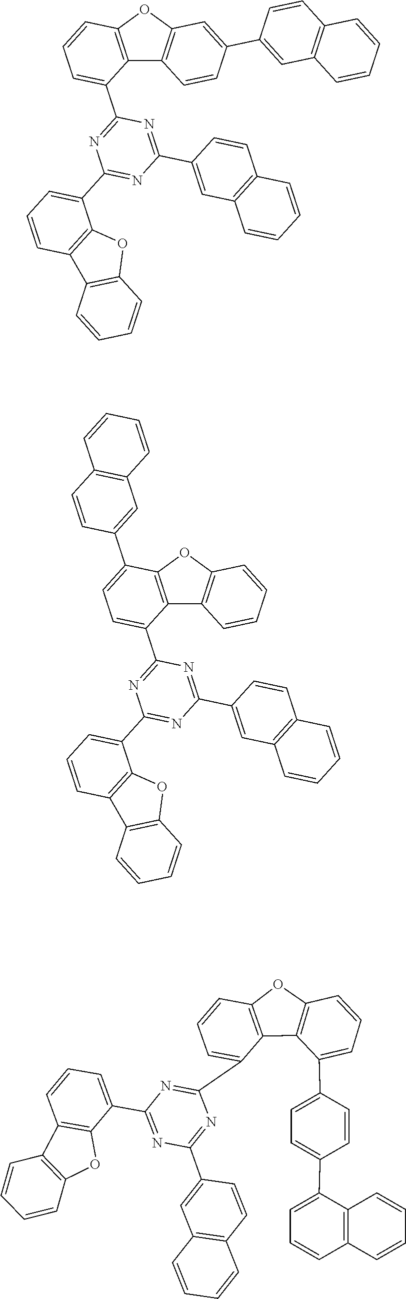 [SMILES: c1ccc2cc(-c3ccc4c(c3)oc3cccc(-c5nc(-c6ccc7ccccc7c6)nc(-c6cccc7c6oc6ccccc67)n5)c34)ccc2c1 ; c1ccc2cc(-c3nc(-c4cccc5c4oc4ccccc45)nc(-c4ccc(-c5ccc6ccccc6c5)c5oc6ccccc6c45)n3)ccc2c1 ; c1ccc2cc(-c3nc(-c4cccc5c4oc4ccccc45)nc(-c4cccc5oc6cccc(-c7ccc(-c8cccc9ccccc89)cc7)c6c45)n3)ccc2c1]

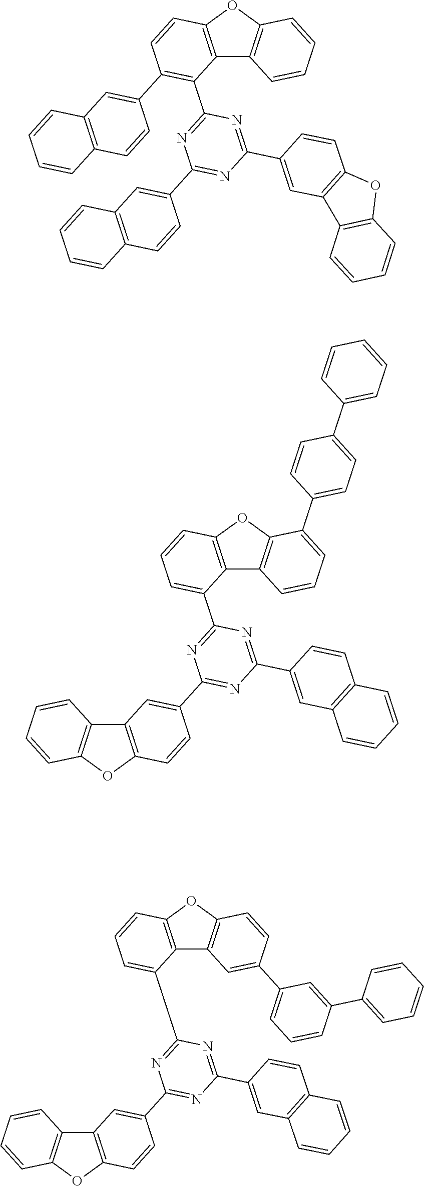 [SMILES: c1ccc2cc(-c3nc(-c4ccc5oc6ccccc6c5c4)nc(-c4c(-c5ccc6ccccc6c5)ccc5oc6ccccc6c45)n3)ccc2c1 ; c1ccc(-c2ccc(-c3cccc4c3oc3cccc(-c5nc(-c6ccc7ccccc7c6)nc(-c6ccc7oc8ccccc8c7c6)n5)c34)cc2)cc1 ; c1ccc(-c2cccc(-c3ccc4oc5cccc(-c6nc(-c7ccc8ccccc8c7)nc(-c7ccc8oc9ccccc9c8c7)n6)c5c4c3)c2)cc1]

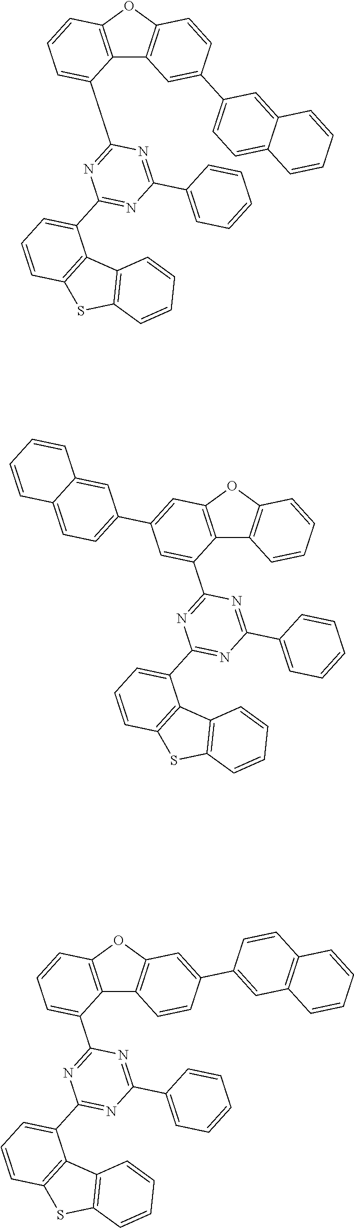 [SMILES: c1ccc(-c2nc(-c3cccc4oc5ccc(-c6ccc7ccccc7c6)cc5c34)nc(-c3cccc4sc5ccccc5c34)n2)cc1 ; c1ccc(-c2nc(-c3cc(-c4ccc5ccccc5c4)cc4oc5ccccc5c34)nc(-c3cccc4sc5ccccc5c34)n2)cc1 ; c1ccc(-c2nc(-c3cccc4oc5cc(-c6ccc7ccccc7c6)ccc5c34)nc(-c3cccc4sc5ccccc5c34)n2)cc1]

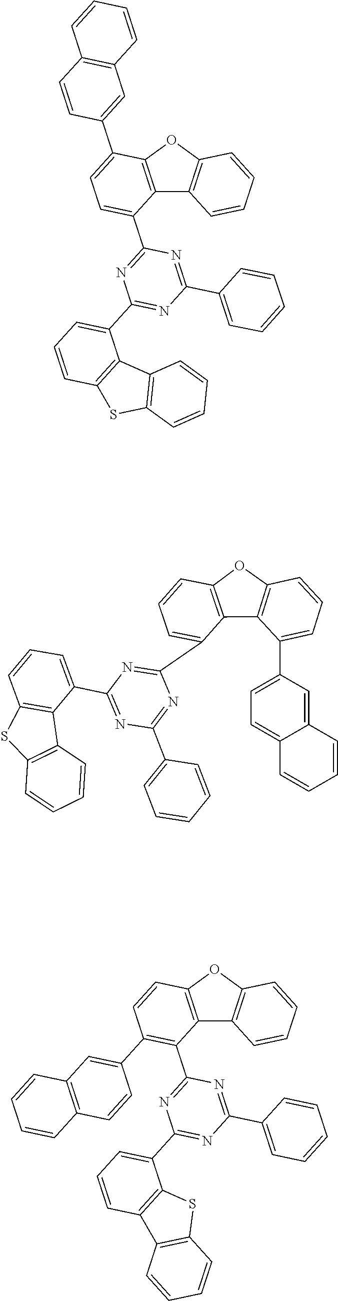 [SMILES: c1ccc(-c2nc(-c3cccc4sc5ccccc5c34)nc(-c3ccc(-c4ccc5ccccc5c4)c4oc5ccccc5c34)n2)cc1 ; c1ccc(-c2nc(-c3cccc4sc5ccccc5c34)nc(-c3cccc4oc5cccc(-c6ccc7ccccc7c6)c5c34)n2)cc1 ; c1ccc(-c2nc(-c3cccc4c3sc3ccccc34)nc(-c3c(-c4ccc5ccccc5c4)ccc4oc5ccccc5c34)n2)cc1]

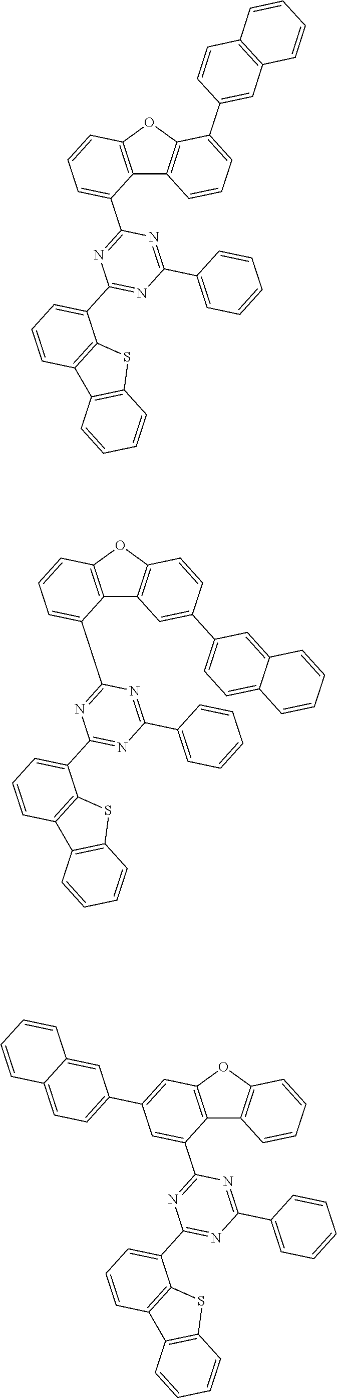 [SMILES: c1ccc(-c2nc(-c3cccc4c3sc3ccccc34)nc(-c3cccc4oc5c(-c6ccc7ccccc7c6)cccc5c34)n2)cc1 ; c1ccc(-c2nc(-c3cccc4c3sc3ccccc34)nc(-c3cccc4oc5ccc(-c6ccc7ccccc7c6)cc5c34)n2)cc1 ; c1ccc(-c2nc(-c3cccc4c3sc3ccccc34)nc(-c3cc(-c4ccc5ccccc5c4)cc4oc5ccccc5c34)n2)cc1]

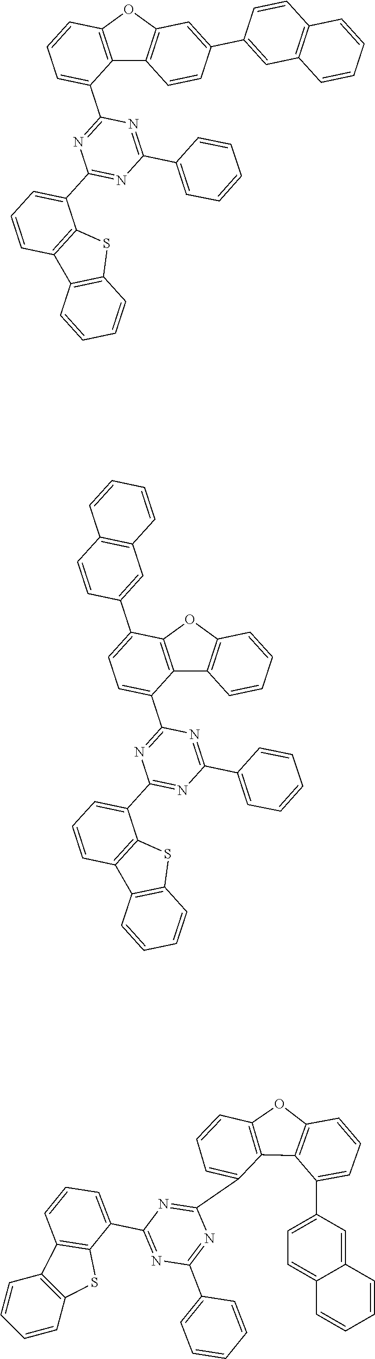 [SMILES: c1ccc(-c2nc(-c3cccc4c3sc3ccccc34)nc(-c3cccc4oc5cc(-c6ccc7ccccc7c6)ccc5c34)n2)cc1 ; c1ccc(-c2nc(-c3cccc4c3sc3ccccc34)nc(-c3ccc(-c4ccc5ccccc5c4)c4oc5ccccc5c34)n2)cc1 ; c1ccc(-c2nc(-c3cccc4c3sc3ccccc34)nc(-c3cccc4oc5cccc(-c6ccc7ccccc7c6)c5c34)n2)cc1]

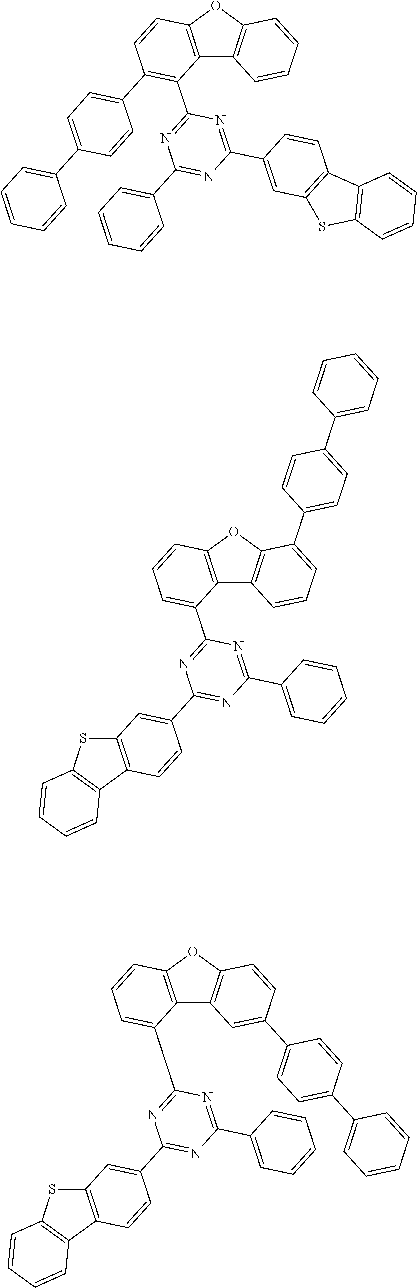 [SMILES: c1ccc(-c2ccc(-c3ccc4oc5ccccc5c4c3-c3nc(-c4ccccc4)nc(-c4ccc5c(c4)sc4ccccc45)n3)cc2)cc1 ; c1ccc(-c2ccc(-c3cccc4c3oc3cccc(-c5nc(-c6ccccc6)nc(-c6ccc7c(c6)sc6ccccc67)n5)c34)cc2)cc1 ; c1ccc(-c2ccc(-c3ccc4oc5cccc(-c6nc(-c7ccccc7)nc(-c7ccc8c(c7)sc7ccccc78)n6)c5c4c3)cc2)cc1]

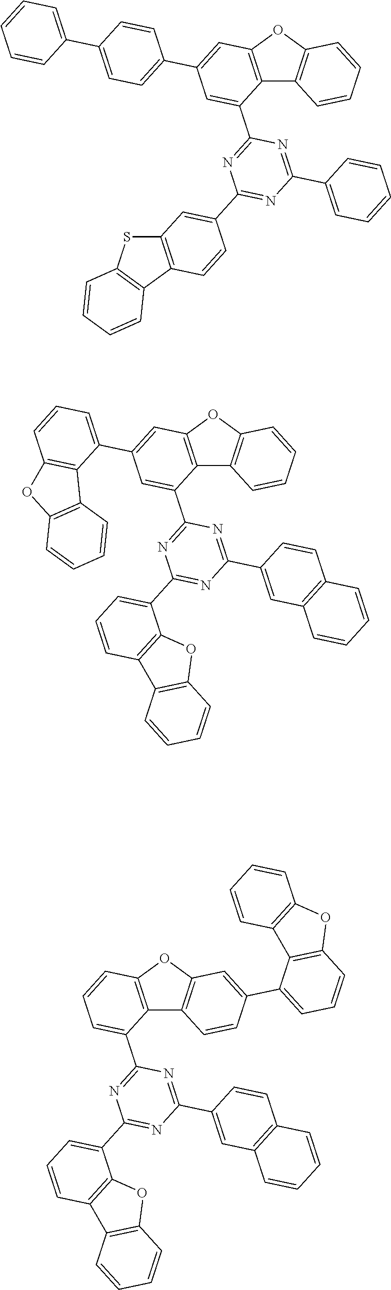 [SMILES: c1ccc(-c2ccc(-c3cc(-c4nc(-c5ccccc5)nc(-c5ccc6c(c5)sc5ccccc56)n4)c4c(c3)oc3ccccc34)cc2)cc1 ; c1ccc2cc(-c3nc(-c4cccc5c4oc4ccccc45)nc(-c4cc(-c5cccc6oc7ccccc7c56)cc5oc6ccccc6c45)n3)ccc2c1 ; c1ccc2cc(-c3nc(-c4cccc5c4oc4ccccc45)nc(-c4cccc5oc6cc(-c7cccc8oc9ccccc9c78)ccc6c45)n3)ccc2c1]

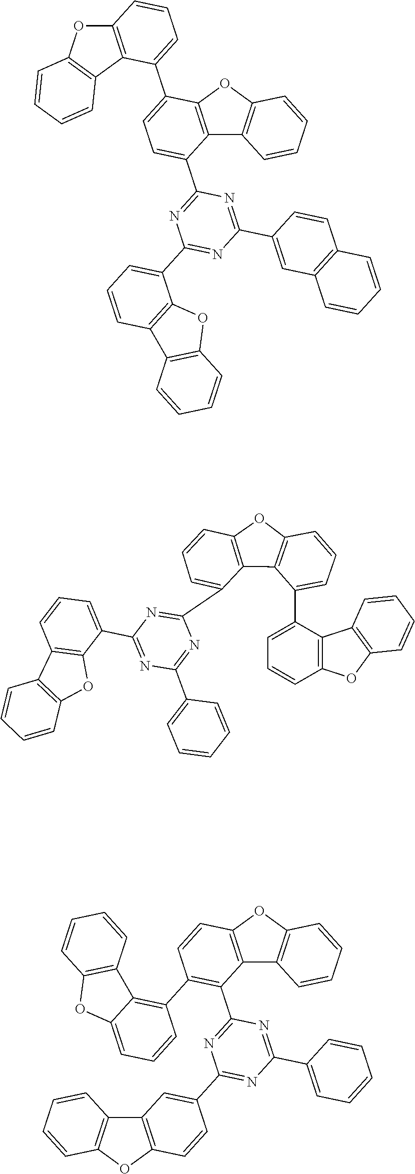 [SMILES: c1ccc2cc(-c3nc(-c4cccc5c4oc4ccccc45)nc(-c4ccc(-c5cccc6oc7ccccc7c56)c5oc6ccccc6c45)n3)ccc2c1 ; c1ccc(-c2nc(-c3cccc4c3oc3ccccc34)nc(-c3cccc4oc5cccc(-c6cccc7oc8ccccc8c67)c5c34)n2)cc1 ; c1ccc(-c2nc(-c3ccc4oc5ccccc5c4c3)nc(-c3c(-c4cccc5oc6ccccc6c45)ccc4oc5ccccc5c34)n2)cc1]

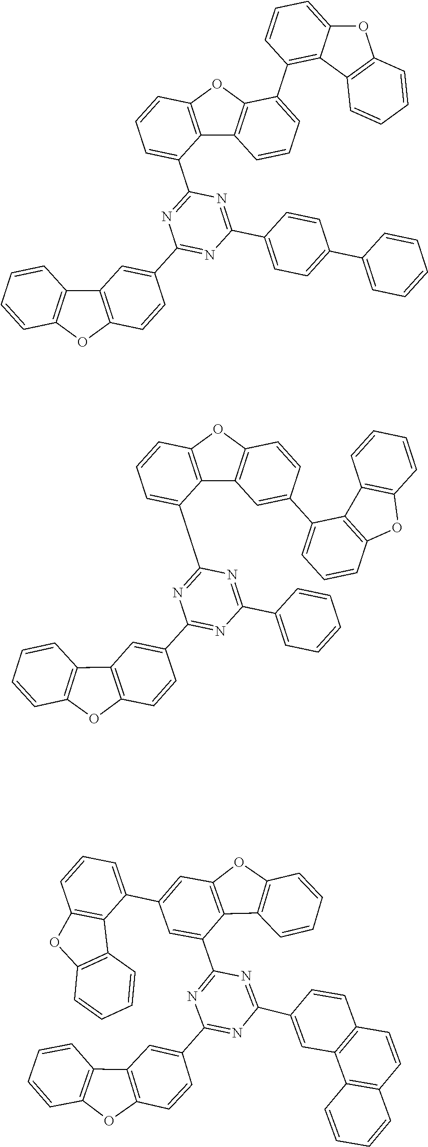 [SMILES: c1ccc(-c2ccc(-c3nc(-c4ccc5oc6ccccc6c5c4)nc(-c4cccc5oc6c(-c7cccc8oc9ccccc9c78)cccc6c45)n3)cc2)cc1 ; c1ccc(-c2nc(-c3ccc4oc5ccccc5c4c3)nc(-c3cccc4oc5ccc(-c6cccc7oc8ccccc8c67)cc5c34)n2)cc1 ; c1ccc2c(c1)ccc1ccc(-c3nc(-c4ccc5oc6ccccc6c5c4)nc(-c4cc(-c5cccc6oc7ccccc7c56)cc5oc6ccccc6c45)n3)cc12]

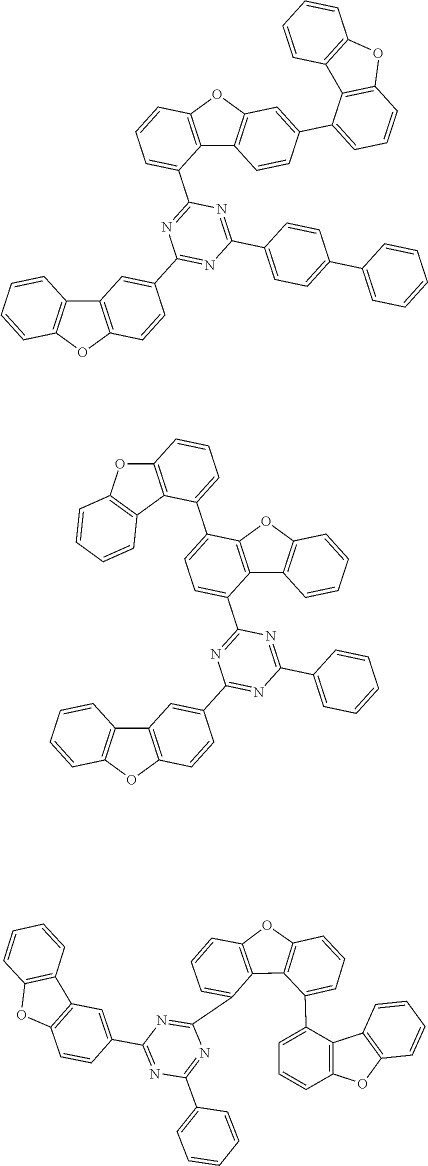 [SMILES: c1ccc(-c2ccc(-c3nc(-c4ccc5oc6ccccc6c5c4)nc(-c4cccc5oc6cc(-c7cccc8oc9ccccc9c78)ccc6c45)n3)cc2)cc1 ; c1ccc(-c2nc(-c3ccc4oc5ccccc5c4c3)nc(-c3ccc(-c4cccc5oc6ccccc6c45)c4oc5ccccc5c34)n2)cc1 ; c1ccc(-c2nc(-c3ccc4oc5ccccc5c4c3)nc(-c3cccc4oc5cccc(-c6cccc7oc8ccccc8c67)c5c34)n2)cc1]

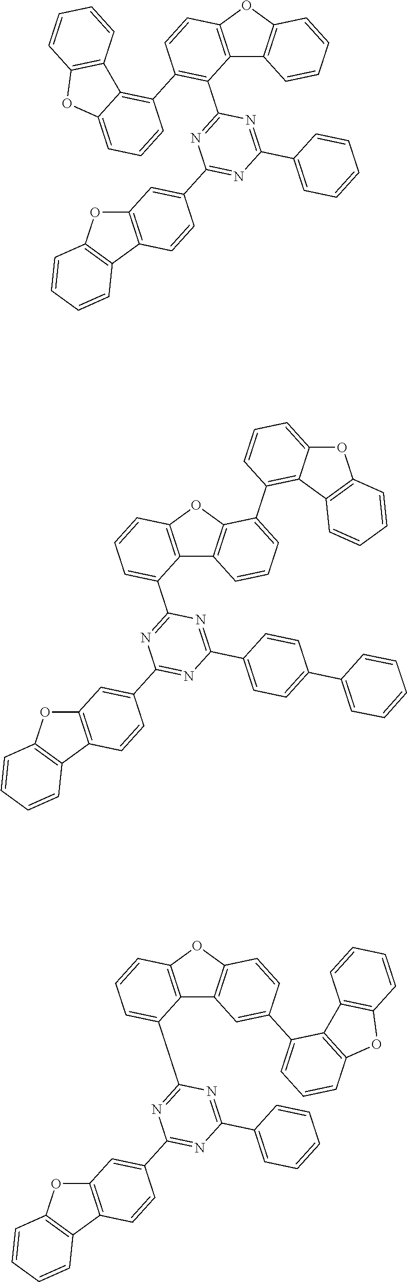 [SMILES: c1ccc(-c2nc(-c3ccc4c(c3)oc3ccccc34)nc(-c3c(-c4cccc5oc6ccccc6c45)ccc4oc5ccccc5c34)n2)cc1 ; c1ccc(-c2ccc(-c3nc(-c4ccc5c(c4)oc4ccccc45)nc(-c4cccc5oc6c(-c7cccc8oc9ccccc9c78)cccc6c45)n3)cc2)cc1 ; c1ccc(-c2nc(-c3ccc4c(c3)oc3ccccc34)nc(-c3cccc4oc5ccc(-c6cccc7oc8ccccc8c67)cc5c34)n2)cc1]

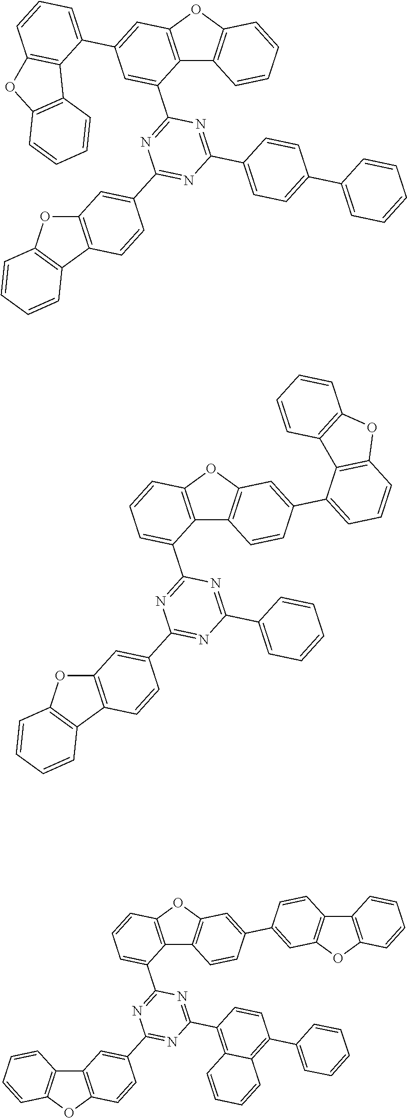 [SMILES: c1ccc(-c2ccc(-c3nc(-c4ccc5c(c4)oc4ccccc45)nc(-c4cc(-c5cccc6oc7ccccc7c56)cc5oc6ccccc6c45)n3)cc2)cc1 ; c1ccc(-c2nc(-c3ccc4c(c3)oc3ccccc34)nc(-c3cccc4oc5cc(-c6cccc7oc8ccccc8c67)ccc5c34)n2)cc1 ; c1ccc(-c2ccc(-c3nc(-c4ccc5oc6ccccc6c5c4)nc(-c4cccc5oc6cc(-c7ccc8c(c7)oc7ccccc78)ccc6c45)n3)c3ccccc23)cc1]

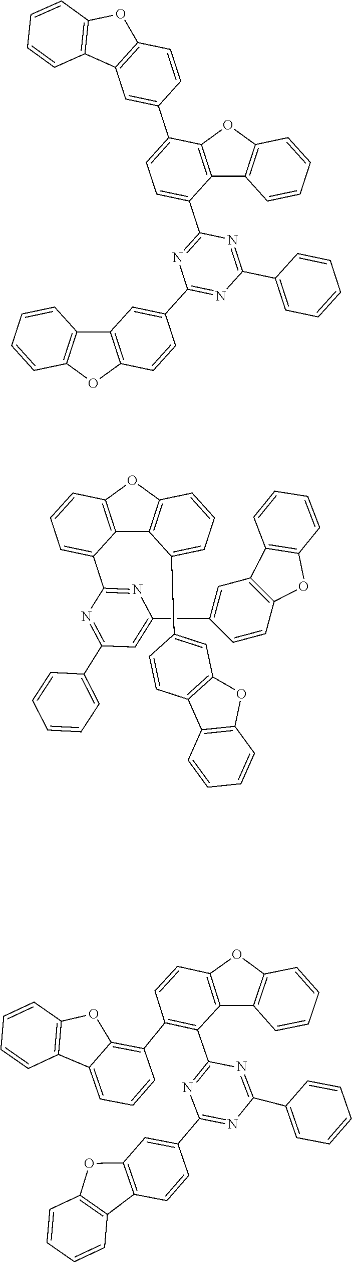 [SMILES: c1ccc(-c2nc(-c3ccc4oc5ccccc5c4c3)nc(-c3ccc(-c4ccc5oc6ccccc6c5c4)c4oc5ccccc5c34)n2)cc1 ; c1ccc(-c2cc(-c3ccc4oc5ccccc5c4c3)nc(-c3cccc4oc5cccc(-c6ccc7c(c6)oc6ccccc67)c5c34)n2)cc1 ; c1ccc(-c2nc(-c3ccc4c(c3)oc3ccccc34)nc(-c3c(-c4cccc5c4oc4ccccc45)ccc4oc5ccccc5c34)n2)cc1]

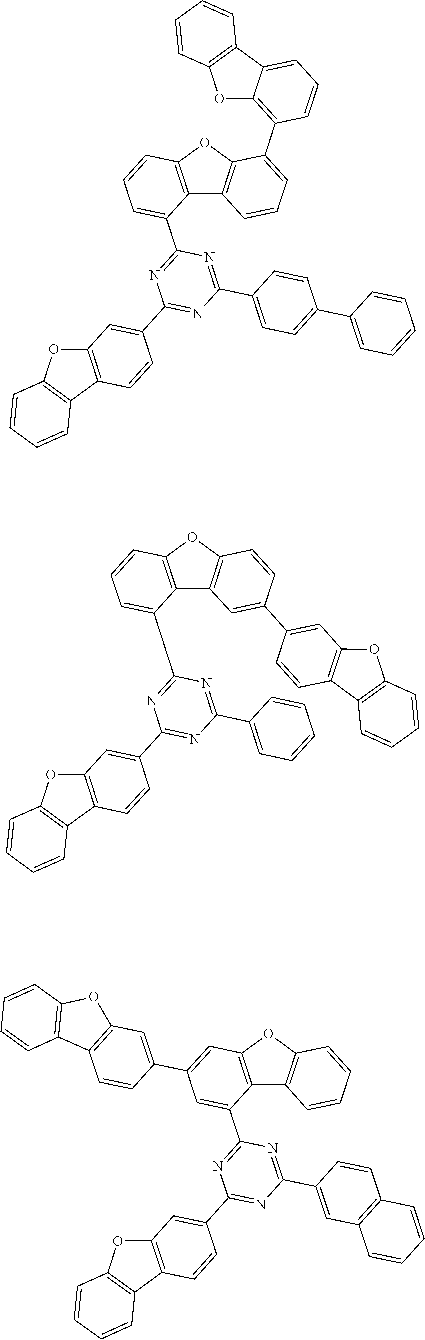 [SMILES: c1ccc(-c2ccc(-c3nc(-c4ccc5c(c4)oc4ccccc45)nc(-c4cccc5oc6c(-c7cccc8c7oc7ccccc78)cccc6c45)n3)cc2)cc1 ; c1ccc(-c2nc(-c3ccc4c(c3)oc3ccccc34)nc(-c3cccc4oc5ccc(-c6ccc7c(c6)oc6ccccc67)cc5c34)n2)cc1 ; c1ccc2cc(-c3nc(-c4ccc5c(c4)oc4ccccc45)nc(-c4cc(-c5ccc6c(c5)oc5ccccc56)cc5oc6ccccc6c45)n3)ccc2c1]

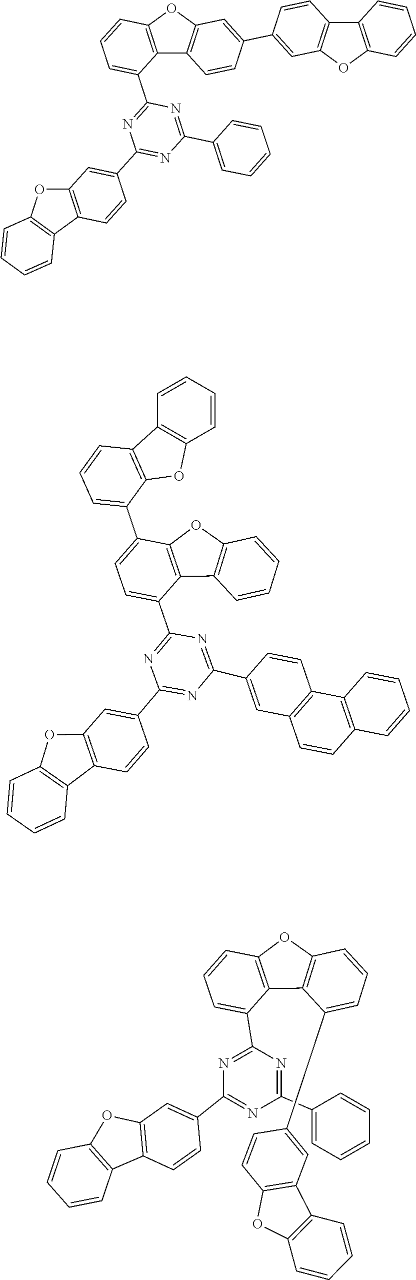 [SMILES: c1ccc(-c2nc(-c3ccc4c(c3)oc3ccccc34)nc(-c3cccc4oc5cc(-c6ccc7c(c6)oc6ccccc67)ccc5c34)n2)cc1 ; c1ccc2c(c1)ccc1cc(-c3nc(-c4ccc5c(c4)oc4ccccc45)nc(-c4ccc(-c5cccc6c5oc5ccccc56)c5oc6ccccc6c45)n3)ccc12 ; c1ccc(-c2nc(-c3ccc4c(c3)oc3ccccc34)nc(-c3cccc4oc5cccc(-c6ccc7oc8ccccc8c7c6)c5c34)n2)cc1]

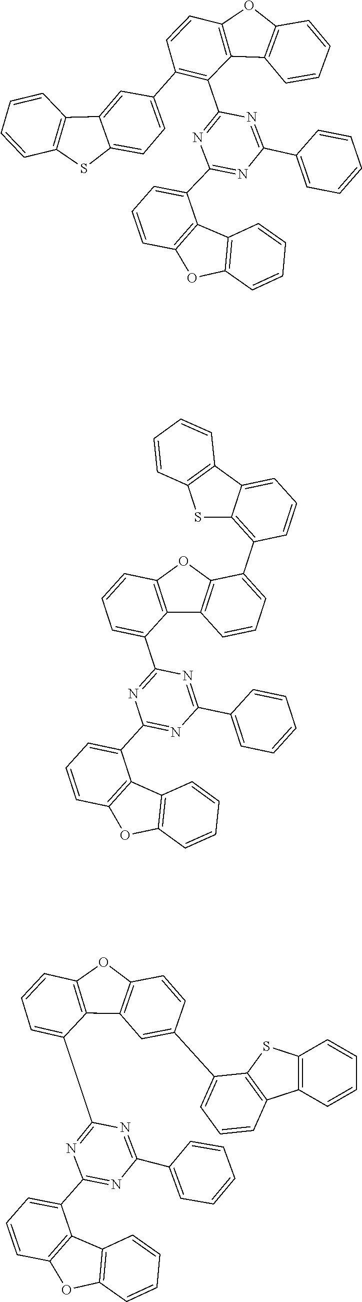 [SMILES: c1ccc(-c2nc(-c3cccc4oc5ccccc5c34)nc(-c3c(-c4ccc5sc6ccccc6c5c4)ccc4oc5ccccc5c34)n2)cc1 ; c1ccc(-c2nc(-c3cccc4oc5ccccc5c34)nc(-c3cccc4oc5c(-c6cccc7c6sc6ccccc67)cccc5c34)n2)cc1 ; c1ccc(-c2nc(-c3cccc4oc5ccccc5c34)nc(-c3cccc4oc5ccc(-c6cccc7c6sc6ccccc67)cc5c34)n2)cc1]

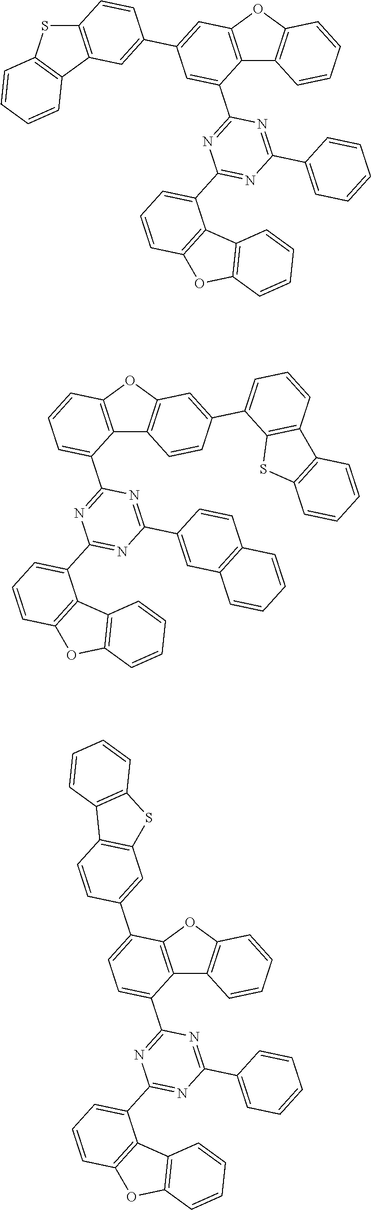 [SMILES: c1ccc(-c2nc(-c3cccc4oc5ccccc5c34)nc(-c3cc(-c4ccc5sc6ccccc6c5c4)cc4oc5ccccc5c34)n2)cc1 ; c1ccc2cc(-c3nc(-c4cccc5oc6ccccc6c45)nc(-c4cccc5oc6cc(-c7cccc8c7sc7ccccc78)ccc6c45)n3)ccc2c1 ; c1ccc(-c2nc(-c3cccc4oc5ccccc5c34)nc(-c3ccc(-c4ccc5c(c4)sc4ccccc45)c4oc5ccccc5c34)n2)cc1]

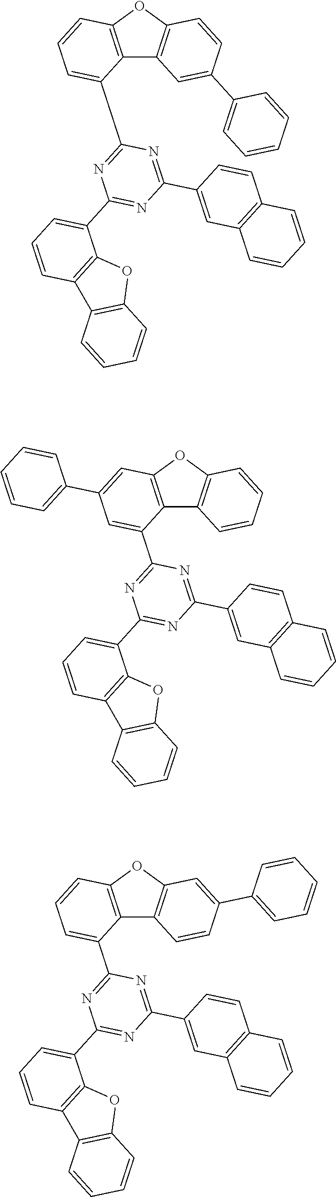 [SMILES: c1ccc(-c2ccc3oc4cccc(-c5nc(-c6ccc7ccccc7c6)nc(-c6cccc7c6oc6ccccc67)n5)c4c3c2)cc1 ; c1ccc(-c2cc(-c3nc(-c4ccc5ccccc5c4)nc(-c4cccc5c4oc4ccccc45)n3)c3c(c2)oc2ccccc23)cc1 ; c1ccc(-c2ccc3c(c2)oc2cccc(-c4nc(-c5ccc6ccccc6c5)nc(-c5cccc6c5oc5ccccc56)n4)c23)cc1]

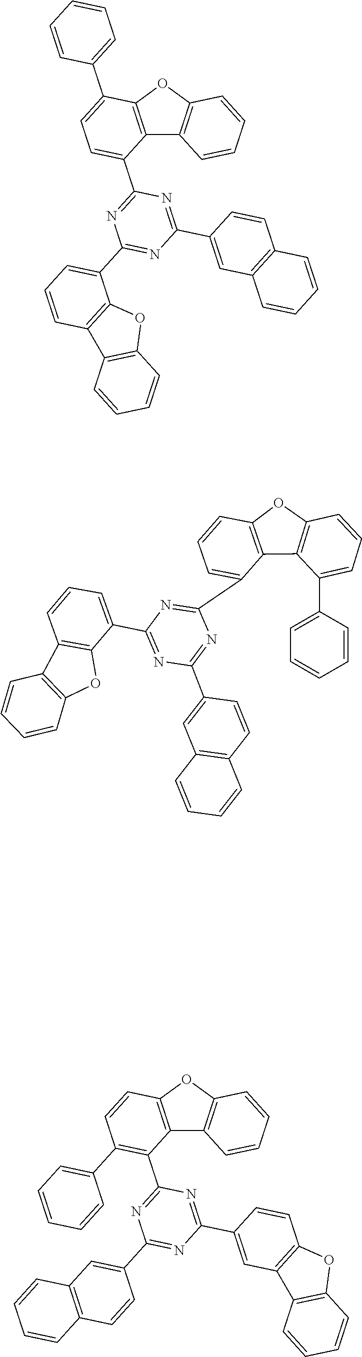 [SMILES: c1ccc(-c2ccc(-c3nc(-c4ccc5ccccc5c4)nc(-c4cccc5c4oc4ccccc45)n3)c3c2oc2ccccc23)cc1 ; c1ccc(-c2cccc3oc4cccc(-c5nc(-c6ccc7ccccc7c6)nc(-c6cccc7c6oc6ccccc67)n5)c4c23)cc1 ; c1ccc(-c2ccc3oc4ccccc4c3c2-c2nc(-c3ccc4ccccc4c3)nc(-c3ccc4oc5ccccc5c4c3)n2)cc1]

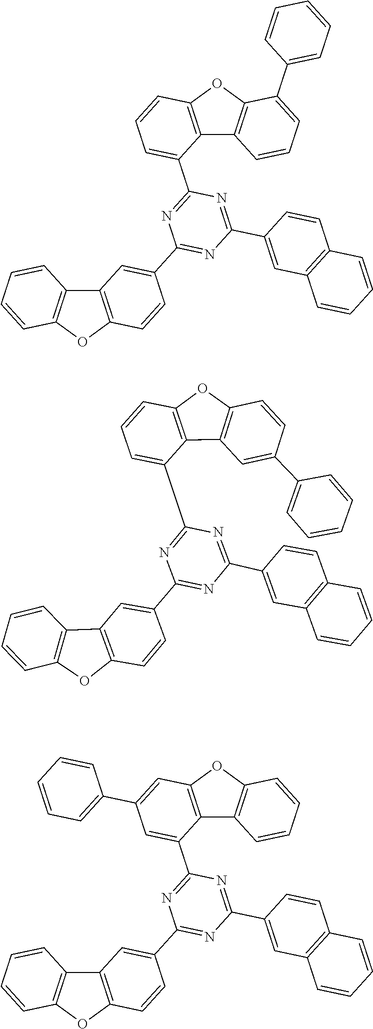 [SMILES: c1ccc(-c2cccc3c2oc2cccc(-c4nc(-c5ccc6ccccc6c5)nc(-c5ccc6oc7ccccc7c6c5)n4)c23)cc1 ; c1ccc(-c2ccc3oc4cccc(-c5nc(-c6ccc7ccccc7c6)nc(-c6ccc7oc8ccccc8c7c6)n5)c4c3c2)cc1 ; c1ccc(-c2cc(-c3nc(-c4ccc5ccccc5c4)nc(-c4ccc5oc6ccccc6c5c4)n3)c3c(c2)oc2ccccc23)cc1]

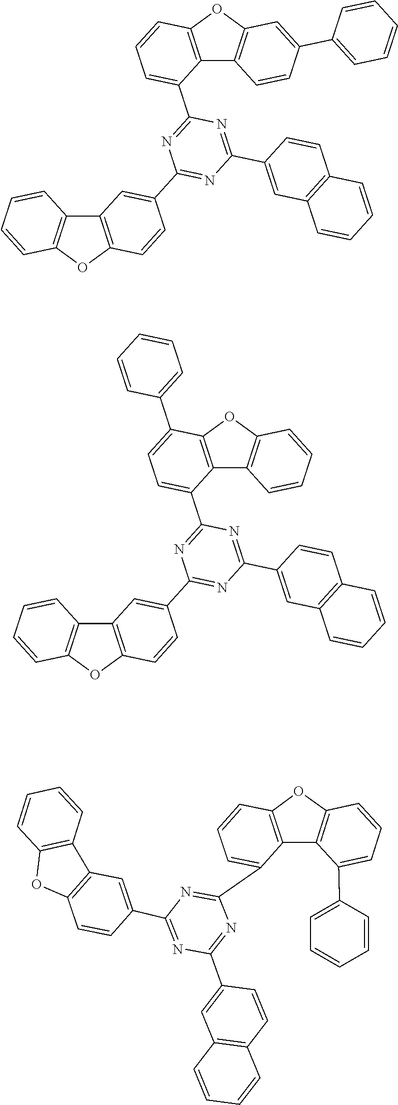 [SMILES: c1ccc(-c2ccc3c(c2)oc2cccc(-c4nc(-c5ccc6ccccc6c5)nc(-c5ccc6oc7ccccc7c6c5)n4)c23)cc1 ; c1ccc(-c2ccc(-c3nc(-c4ccc5ccccc5c4)nc(-c4ccc5oc6ccccc6c5c4)n3)c3c2oc2ccccc23)cc1 ; c1ccc(-c2cccc3oc4cccc(-c5nc(-c6ccc7ccccc7c6)nc(-c6ccc7oc8ccccc8c7c6)n5)c4c23)cc1]

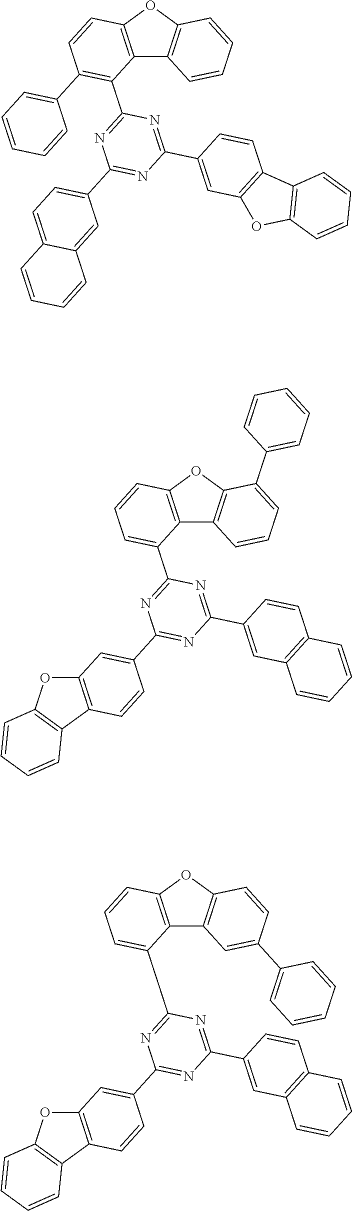 [SMILES: c1ccc(-c2ccc3oc4ccccc4c3c2-c2nc(-c3ccc4ccccc4c3)nc(-c3ccc4c(c3)oc3ccccc34)n2)cc1 ; c1ccc(-c2cccc3c2oc2cccc(-c4nc(-c5ccc6ccccc6c5)nc(-c5ccc6c(c5)oc5ccccc56)n4)c23)cc1 ; c1ccc(-c2ccc3oc4cccc(-c5nc(-c6ccc7ccccc7c6)nc(-c6ccc7c(c6)oc6ccccc67)n5)c4c3c2)cc1]

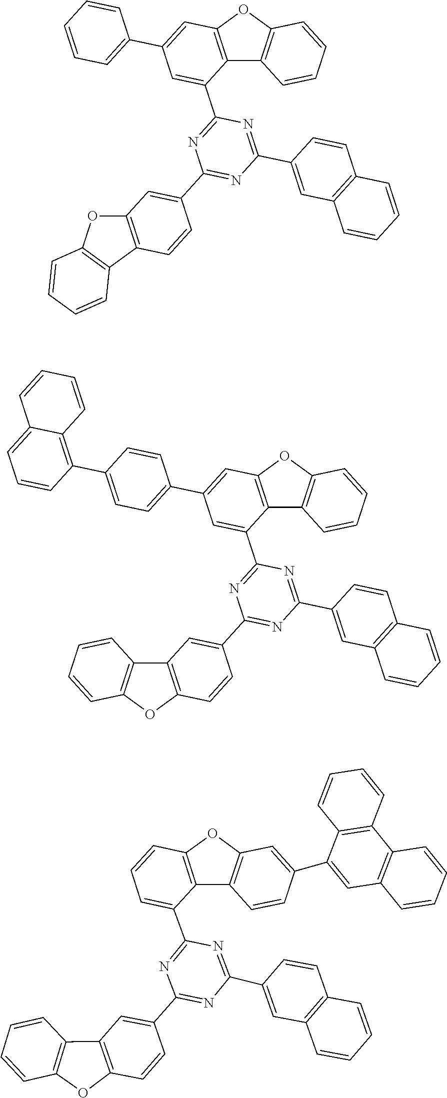 [SMILES: c1ccc(-c2cc(-c3nc(-c4ccc5ccccc5c4)nc(-c4ccc5c(c4)oc4ccccc45)n3)c3c(c2)oc2ccccc23)cc1 ; c1ccc2cc(-c3nc(-c4ccc5oc6ccccc6c5c4)nc(-c4cc(-c5ccc(-c6cccc7ccccc67)cc5)cc5oc6ccccc6c45)n3)ccc2c1 ; c1ccc2cc(-c3nc(-c4ccc5oc6ccccc6c5c4)nc(-c4cccc5oc6cc(-c7cc8ccccc8c8ccccc78)ccc6c45)n3)ccc2c1]

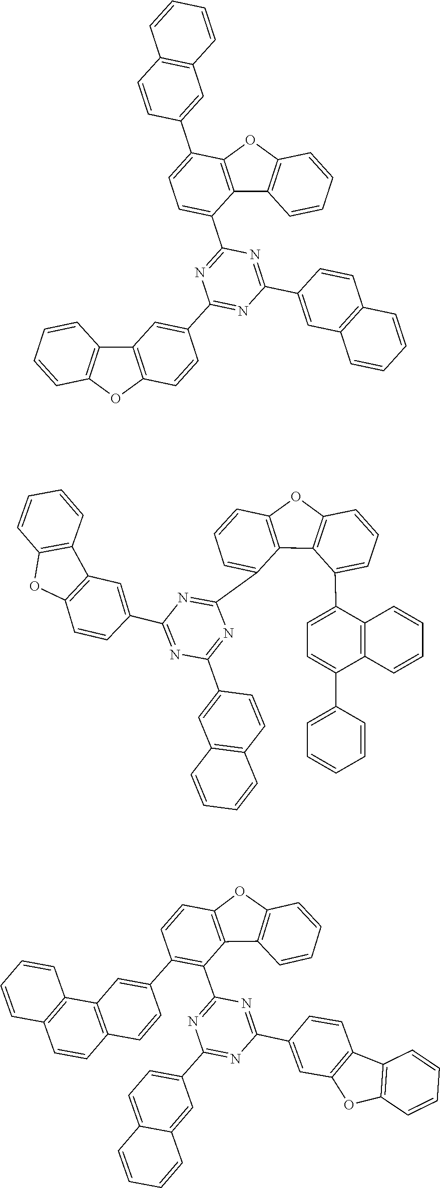 [SMILES: c1ccc2cc(-c3nc(-c4ccc5oc6ccccc6c5c4)nc(-c4ccc(-c5ccc6ccccc6c5)c5oc6ccccc6c45)n3)ccc2c1 ; c1ccc(-c2ccc(-c3cccc4oc5cccc(-c6nc(-c7ccc8ccccc8c7)nc(-c7ccc8oc9ccccc9c8c7)n6)c5c34)c3ccccc23)cc1 ; c1ccc2cc(-c3nc(-c4ccc5c(c4)oc4ccccc45)nc(-c4c(-c5ccc6ccc7ccccc7c6c5)ccc5oc6ccccc6c45)n3)ccc2c1]

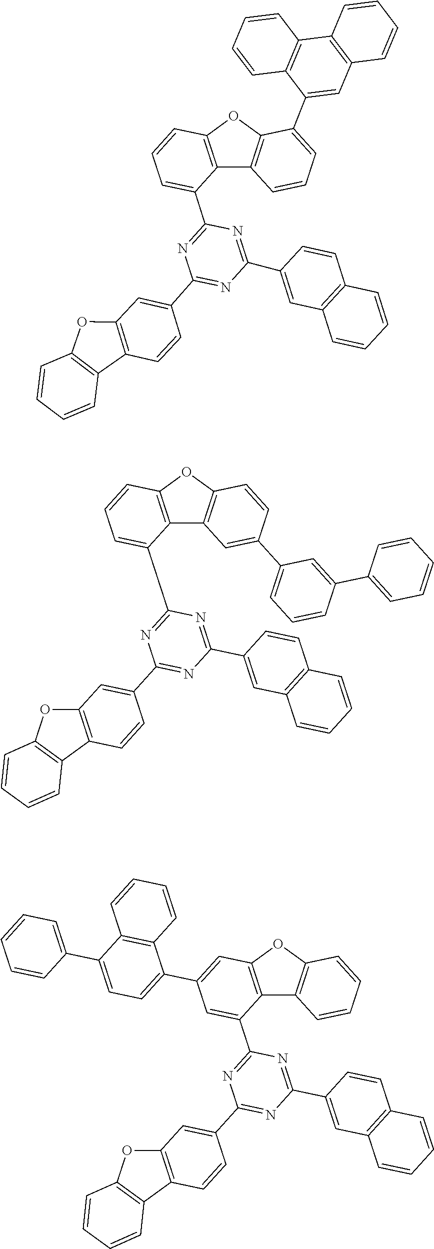 [SMILES: c1ccc2cc(-c3nc(-c4ccc5c(c4)oc4ccccc45)nc(-c4cccc5oc6c(-c7cc8ccccc8c8ccccc78)cccc6c45)n3)ccc2c1 ; c1ccc(-c2cccc(-c3ccc4oc5cccc(-c6nc(-c7ccc8ccccc8c7)nc(-c7ccc8c(c7)oc7ccccc78)n6)c5c4c3)c2)cc1 ; c1ccc(-c2ccc(-c3cc(-c4nc(-c5ccc6ccccc6c5)nc(-c5ccc6c(c5)oc5ccccc56)n4)c4c(c3)oc3ccccc34)c3ccccc23)cc1]

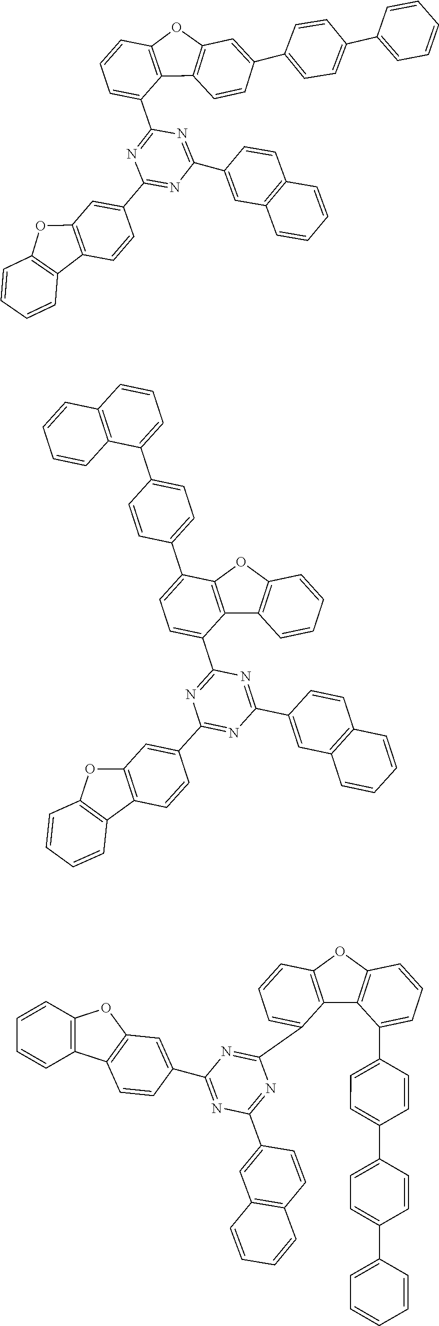 [SMILES: c1ccc(-c2ccc(-c3ccc4c(c3)oc3cccc(-c5nc(-c6ccc7ccccc7c6)nc(-c6ccc7c(c6)oc6ccccc67)n5)c34)cc2)cc1 ; c1ccc2cc(-c3nc(-c4ccc5c(c4)oc4ccccc45)nc(-c4ccc(-c5ccc(-c6cccc7ccccc67)cc5)c5oc6ccccc6c45)n3)ccc2c1 ; c1ccc(-c2ccc(-c3ccc(-c4cccc5oc6cccc(-c7nc(-c8ccc9ccccc9c8)nc(-c8ccc9c(c8)oc8ccccc89)n7)c6c45)cc3)cc2)cc1]

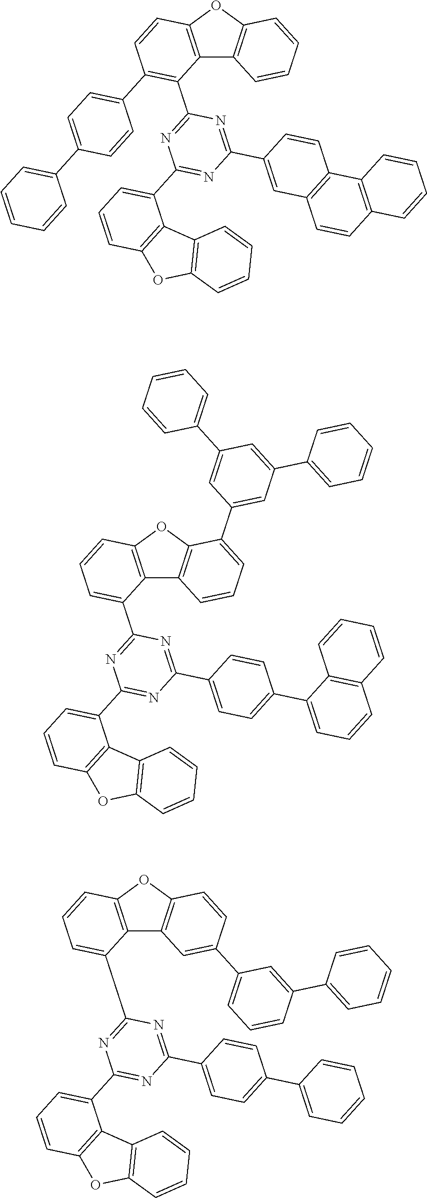 [SMILES: c1ccc(-c2ccc(-c3ccc4oc5ccccc5c4c3-c3nc(-c4ccc5c(ccc6ccccc65)c4)nc(-c4cccc5oc6ccccc6c45)n3)cc2)cc1 ; c1ccc(-c2cc(-c3ccccc3)cc(-c3cccc4c3oc3cccc(-c5nc(-c6ccc(-c7cccc8ccccc78)cc6)nc(-c6cccc7oc8ccccc8c67)n5)c34)c2)cc1 ; c1ccc(-c2ccc(-c3nc(-c4cccc5oc6ccccc6c45)nc(-c4cccc5oc6ccc(-c7cccc(-c8ccccc8)c7)cc6c45)n3)cc2)cc1]

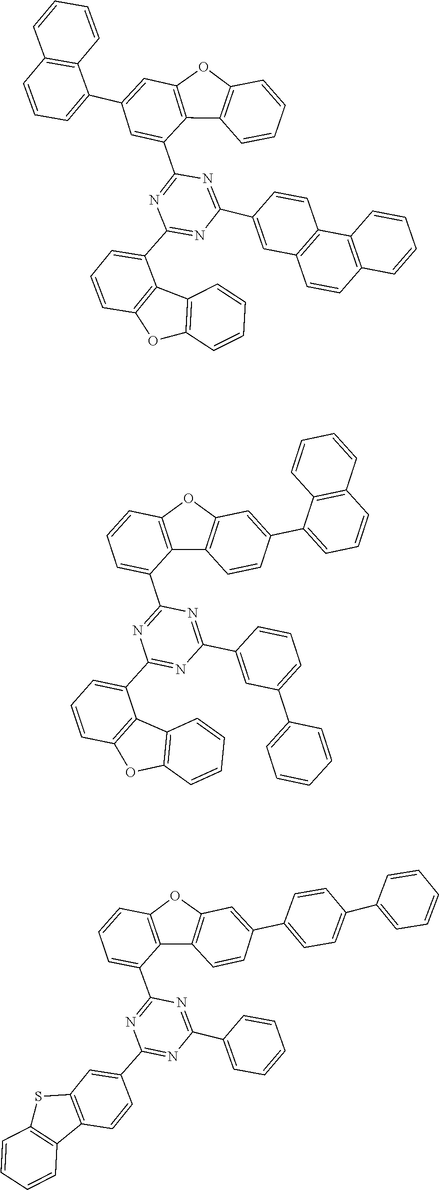 [SMILES: c1ccc2c(-c3cc(-c4nc(-c5ccc6c(ccc7ccccc76)c5)nc(-c5cccc6oc7ccccc7c56)n4)c4c(c3)oc3ccccc34)cccc2c1 ; c1ccc(-c2cccc(-c3nc(-c4cccc5oc6ccccc6c45)nc(-c4cccc5oc6cc(-c7cccc8ccccc78)ccc6c45)n3)c2)cc1 ; c1ccc(-c2ccc(-c3ccc4c(c3)oc3cccc(-c5nc(-c6ccccc6)nc(-c6ccc7c(c6)sc6ccccc67)n5)c34)cc2)cc1]

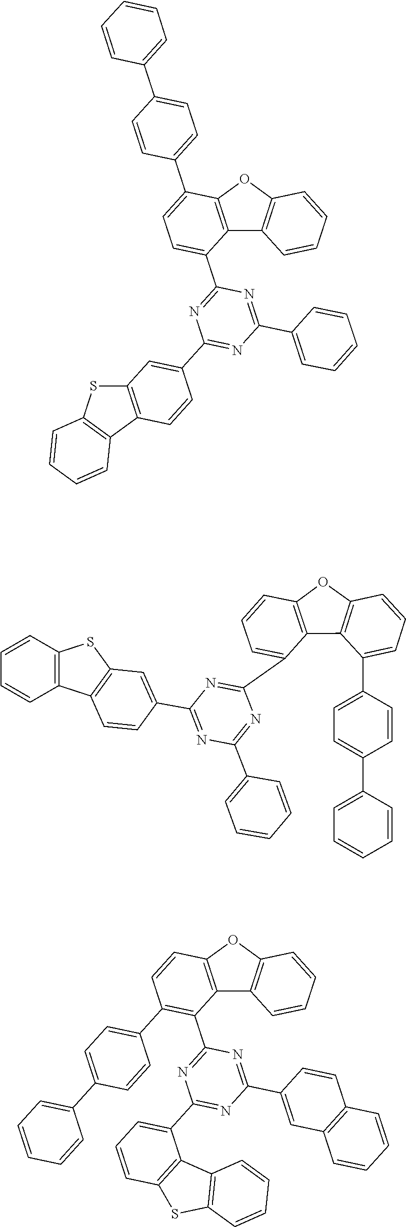 [SMILES: c1ccc(-c2ccc(-c3ccc(-c4nc(-c5ccccc5)nc(-c5ccc6c(c5)sc5ccccc56)n4)c4c3oc3ccccc34)cc2)cc1 ; c1ccc(-c2ccc(-c3cccc4oc5cccc(-c6nc(-c7ccccc7)nc(-c7ccc8c(c7)sc7ccccc78)n6)c5c34)cc2)cc1 ; c1ccc(-c2ccc(-c3ccc4oc5ccccc5c4c3-c3nc(-c4ccc5ccccc5c4)nc(-c4cccc5sc6ccccc6c45)n3)cc2)cc1]

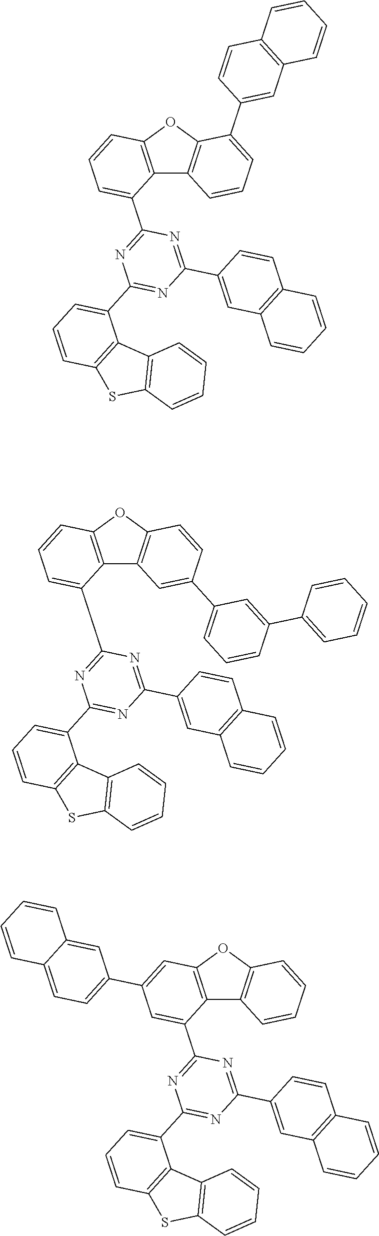 [SMILES: c1ccc2cc(-c3nc(-c4cccc5oc6c(-c7ccc8ccccc8c7)cccc6c45)nc(-c4cccc5sc6ccccc6c45)n3)ccc2c1 ; c1ccc(-c2cccc(-c3ccc4oc5cccc(-c6nc(-c7ccc8ccccc8c7)nc(-c7cccc8sc9ccccc9c78)n6)c5c4c3)c2)cc1 ; c1ccc2cc(-c3cc(-c4nc(-c5ccc6ccccc6c5)nc(-c5cccc6sc7ccccc7c56)n4)c4c(c3)oc3ccccc34)ccc2c1]

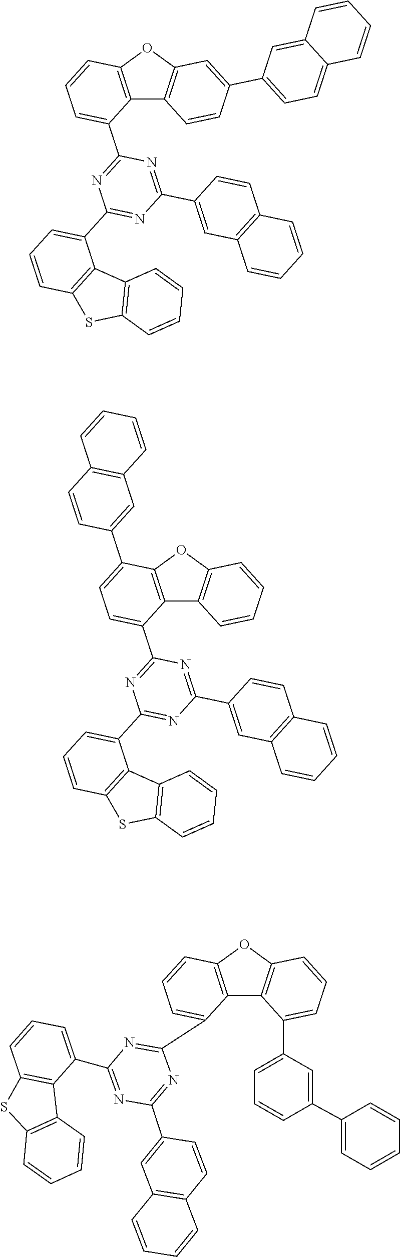 [SMILES: c1ccc2cc(-c3ccc4c(c3)oc3cccc(-c5nc(-c6ccc7ccccc7c6)nc(-c6cccc7sc8ccccc8c67)n5)c34)ccc2c1 ; c1ccc2cc(-c3nc(-c4cccc5sc6ccccc6c45)nc(-c4ccc(-c5ccc6ccccc6c5)c5oc6ccccc6c45)n3)ccc2c1 ; c1ccc(-c2cccc(-c3cccc4oc5cccc(-c6nc(-c7ccc8ccccc8c7)nc(-c7cccc8sc9ccccc9c78)n6)c5c34)c2)cc1]

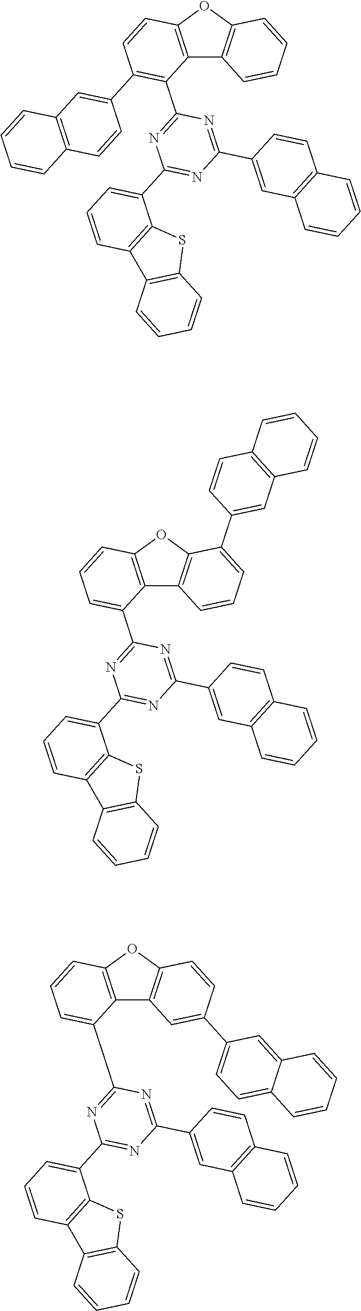 [SMILES: c1ccc2cc(-c3nc(-c4cccc5c4sc4ccccc45)nc(-c4c(-c5ccc6ccccc6c5)ccc5oc6ccccc6c45)n3)ccc2c1 ; c1ccc2cc(-c3nc(-c4cccc5c4sc4ccccc45)nc(-c4cccc5oc6c(-c7ccc8ccccc8c7)cccc6c45)n3)ccc2c1 ; c1ccc2cc(-c3ccc4oc5cccc(-c6nc(-c7ccc8ccccc8c7)nc(-c7cccc8c7sc7ccccc78)n6)c5c4c3)ccc2c1]

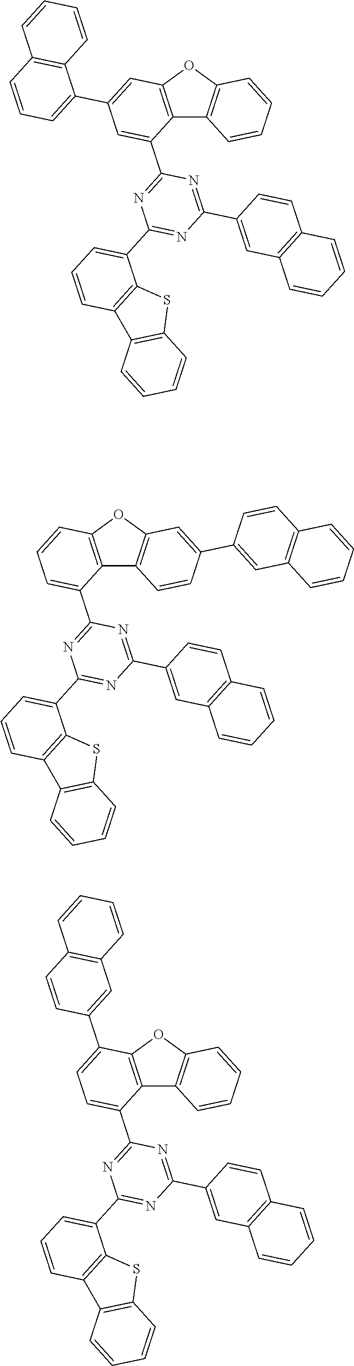 [SMILES: c1ccc2cc(-c3nc(-c4cccc5c4sc4ccccc45)nc(-c4cc(-c5cccc6ccccc56)cc5oc6ccccc6c45)n3)ccc2c1 ; c1ccc2cc(-c3ccc4c(c3)oc3cccc(-c5nc(-c6ccc7ccccc7c6)nc(-c6cccc7c6sc6ccccc67)n5)c34)ccc2c1 ; c1ccc2cc(-c3nc(-c4cccc5c4sc4ccccc45)nc(-c4ccc(-c5ccc6ccccc6c5)c5oc6ccccc6c45)n3)ccc2c1]

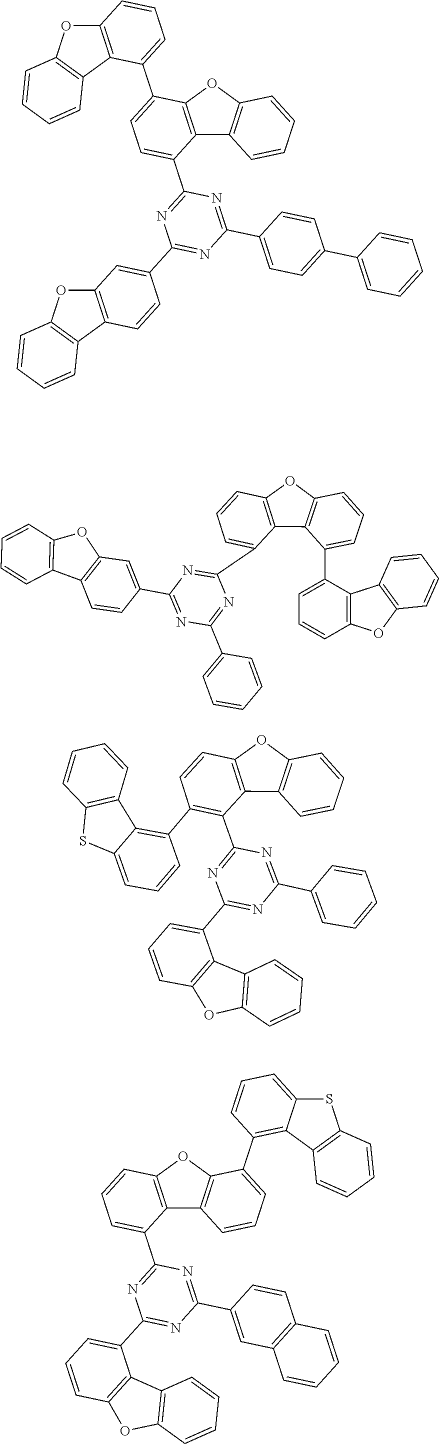

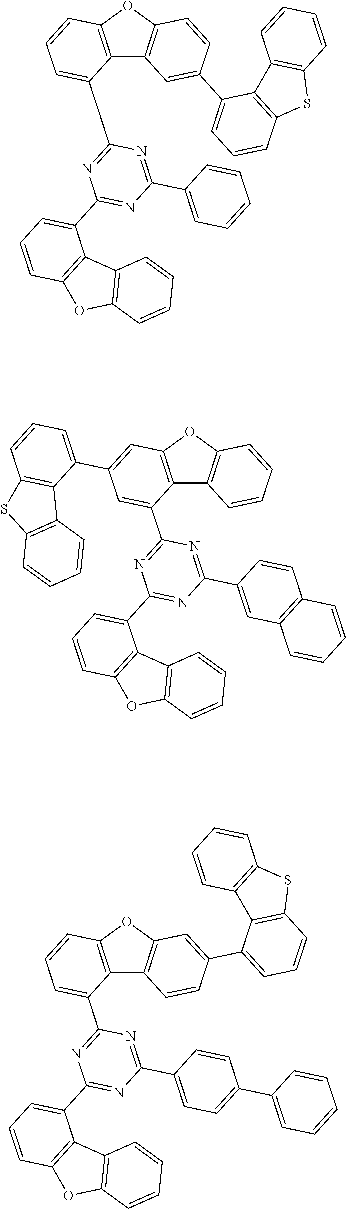 [SMILES: c1ccc(-c2nc(-c3cccc4oc5ccccc5c34)nc(-c3cccc4oc5ccc(-c6cccc7sc8ccccc8c67)cc5c34)n2)cc1 ; c1ccc2cc(-c3nc(-c4cccc5oc6ccccc6c45)nc(-c4cc(-c5cccc6sc7ccccc7c56)cc5oc6ccccc6c45)n3)ccc2c1 ; c1ccc(-c2ccc(-c3nc(-c4cccc5oc6ccccc6c45)nc(-c4cccc5oc6cc(-c7cccc8sc9ccccc9c78)ccc6c45)n3)cc2)cc1]

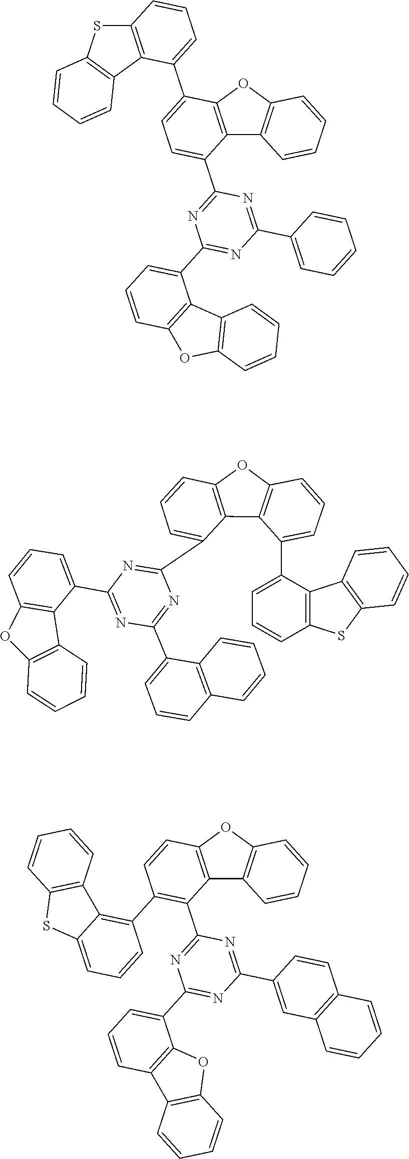 [SMILES: c1ccc(-c2nc(-c3cccc4oc5ccccc5c34)nc(-c3ccc(-c4cccc5sc6ccccc6c45)c4oc5ccccc5c34)n2)cc1 ; c1ccc2c(-c3nc(-c4cccc5oc6ccccc6c45)nc(-c4cccc5oc6cccc(-c7cccc8sc9ccccc9c78)c6c45)n3)cccc2c1 ; c1ccc2cc(-c3nc(-c4cccc5c4oc4ccccc45)nc(-c4c(-c5cccc6sc7ccccc7c56)ccc5oc6ccccc6c45)n3)ccc2c1]

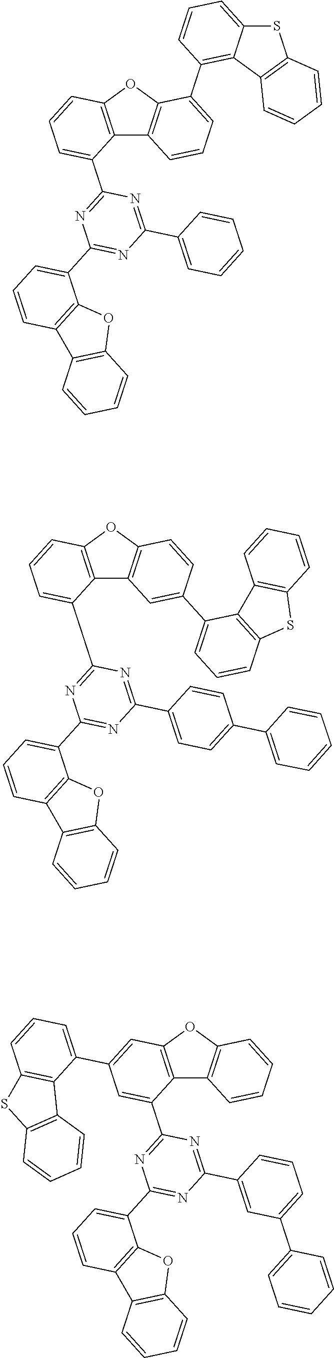 [SMILES: c1ccc(-c2nc(-c3cccc4c3oc3ccccc34)nc(-c3cccc4oc5c(-c6cccc7sc8ccccc8c67)cccc5c34)n2)cc1 ; c1ccc(-c2ccc(-c3nc(-c4cccc5c4oc4ccccc45)nc(-c4cccc5oc6ccc(-c7cccc8sc9ccccc9c78)cc6c45)n3)cc2)cc1 ; c1ccc(-c2cccc(-c3nc(-c4cccc5c4oc4ccccc45)nc(-c4cc(-c5cccc6sc7ccccc7c56)cc5oc6ccccc6c45)n3)c2)cc1]

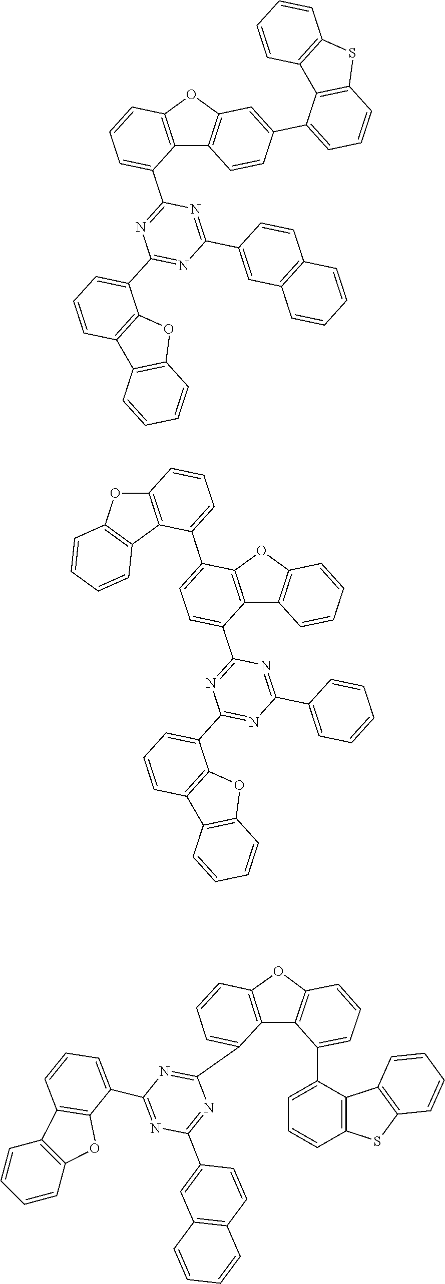 [SMILES: c1ccc2cc(-c3nc(-c4cccc5c4oc4ccccc45)nc(-c4cccc5oc6cc(-c7cccc8sc9ccccc9c78)ccc6c45)n3)ccc2c1 ; c1ccc(-c2nc(-c3cccc4c3oc3ccccc34)nc(-c3ccc(-c4cccc5oc6ccccc6c45)c4oc5ccccc5c34)n2)cc1 ; c1ccc2cc(-c3nc(-c4cccc5c4oc4ccccc45)nc(-c4cccc5oc6cccc(-c7cccc8sc9ccccc9c78)c6c45)n3)ccc2c1]

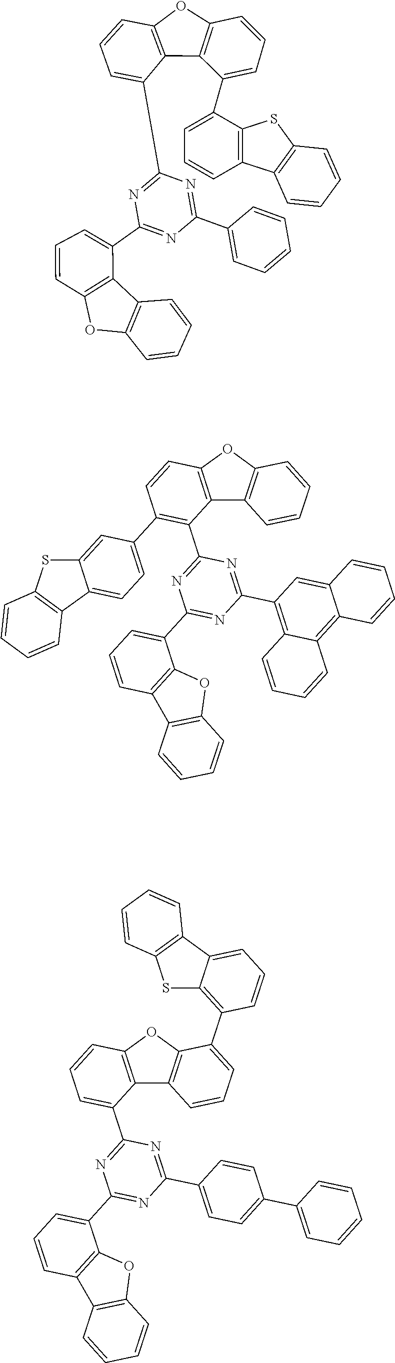 [SMILES: c1ccc(-c2nc(-c3cccc4oc5ccccc5c34)nc(-c3cccc4oc5cccc(-c6cccc7c6sc6ccccc67)c5c34)n2)cc1 ; c1ccc2c(c1)cc(-c1nc(-c3cccc4c3oc3ccccc34)nc(-c3c(-c4ccc5c(c4)sc4ccccc45)ccc4oc5ccccc5c34)n1)c1ccccc12 ; c1ccc(-c2ccc(-c3nc(-c4cccc5c4oc4ccccc45)nc(-c4cccc5oc6c(-c7cccc8c7sc7ccccc78)cccc6c45)n3)cc2)cc1]

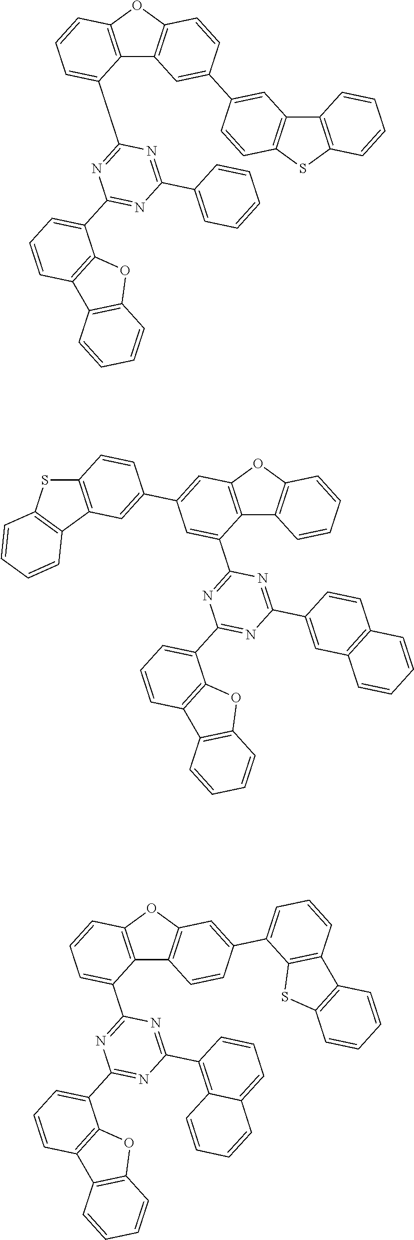 [SMILES: c1ccc(-c2nc(-c3cccc4c3oc3ccccc34)nc(-c3cccc4oc5ccc(-c6ccc7sc8ccccc8c7c6)cc5c34)n2)cc1 ; c1ccc2cc(-c3nc(-c4cccc5c4oc4ccccc45)nc(-c4cc(-c5ccc6sc7ccccc7c6c5)cc5oc6ccccc6c45)n3)ccc2c1 ; c1ccc2c(-c3nc(-c4cccc5c4oc4ccccc45)nc(-c4cccc5oc6cc(-c7cccc8c7sc7ccccc78)ccc6c45)n3)cccc2c1]

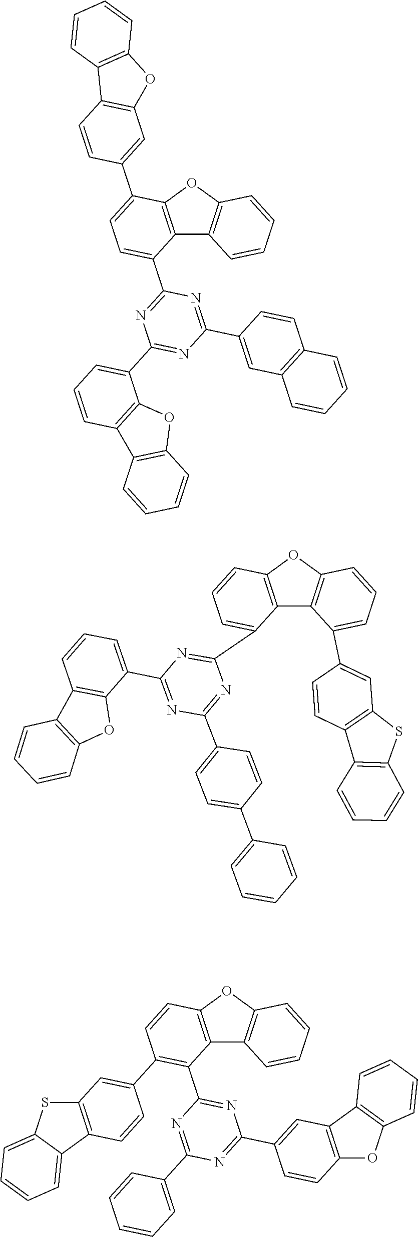 [SMILES: c1ccc2cc(-c3nc(-c4cccc5c4oc4ccccc45)nc(-c4ccc(-c5ccc6c(c5)oc5ccccc56)c5oc6ccccc6c45)n3)ccc2c1 ; c1ccc(-c2ccc(-c3nc(-c4cccc5c4oc4ccccc45)nc(-c4cccc5oc6cccc(-c7ccc8c(c7)sc7ccccc78)c6c45)n3)cc2)cc1 ; c1ccc(-c2nc(-c3ccc4oc5ccccc5c4c3)nc(-c3c(-c4ccc5c(c4)sc4ccccc45)ccc4oc5ccccc5c34)n2)cc1]

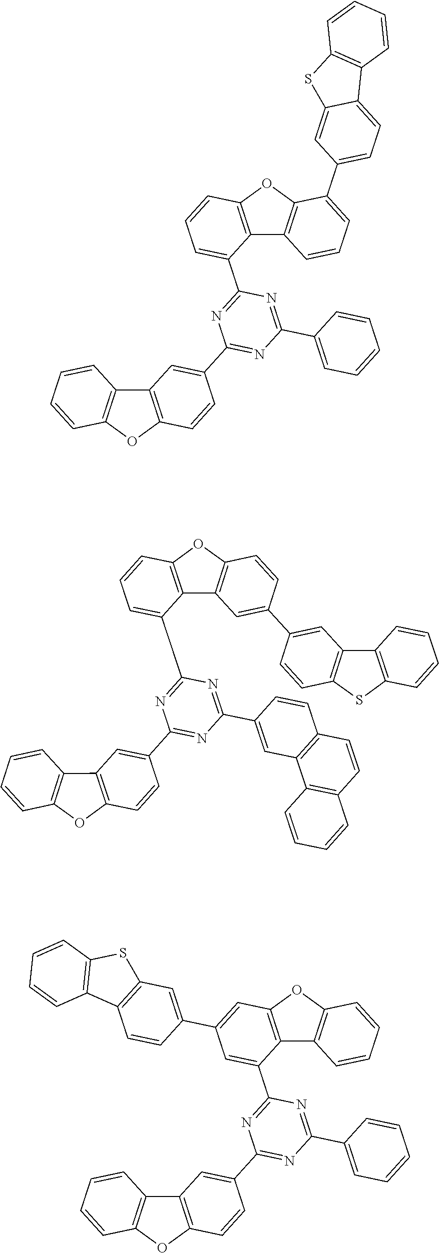 [SMILES: c1ccc(-c2nc(-c3ccc4oc5ccccc5c4c3)nc(-c3cccc4oc5c(-c6ccc7c(c6)sc6ccccc67)cccc5c34)n2)cc1 ; c1ccc2c(c1)ccc1ccc(-c3nc(-c4ccc5oc6ccccc6c5c4)nc(-c4cccc5oc6ccc(-c7ccc8sc9ccccc9c8c7)cc6c45)n3)cc12 ; c1ccc(-c2nc(-c3ccc4oc5ccccc5c4c3)nc(-c3cc(-c4ccc5c(c4)sc4ccccc45)cc4oc5ccccc5c34)n2)cc1]

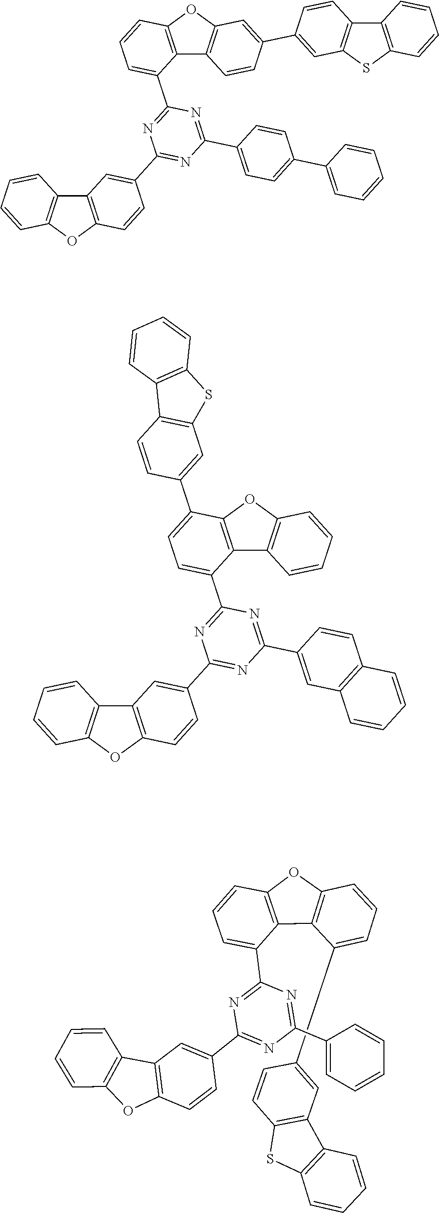 [SMILES: c1ccc(-c2ccc(-c3nc(-c4ccc5oc6ccccc6c5c4)nc(-c4cccc5oc6cc(-c7ccc8c(c7)sc7ccccc78)ccc6c45)n3)cc2)cc1 ; c1ccc2cc(-c3nc(-c4ccc5oc6ccccc6c5c4)nc(-c4ccc(-c5ccc6c(c5)sc5ccccc56)c5oc6ccccc6c45)n3)ccc2c1 ; c1ccc(-c2nc(-c3ccc4oc5ccccc5c4c3)nc(-c3cccc4oc5cccc(-c6ccc7sc8ccccc8c7c6)c5c34)n2)cc1]

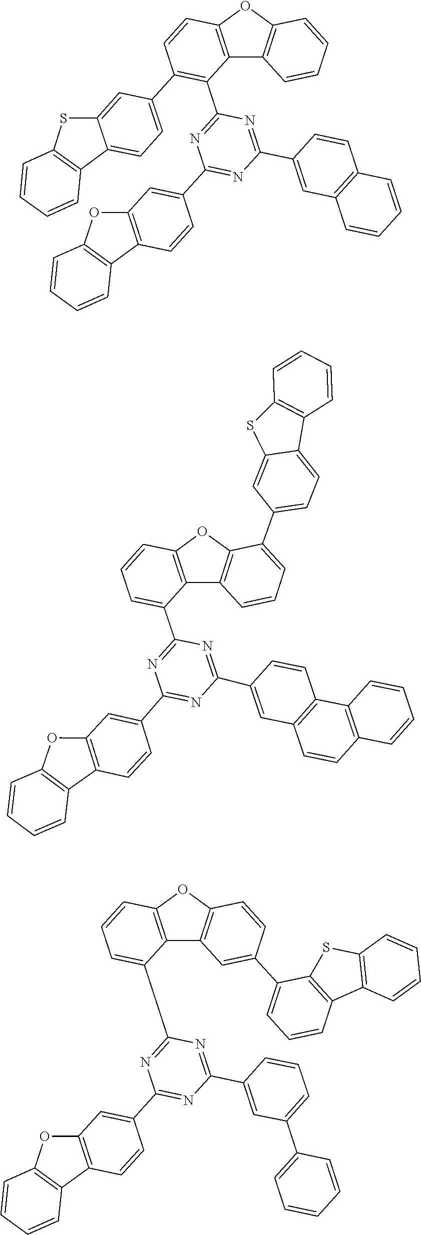 [SMILES: c1ccc2cc(-c3nc(-c4ccc5c(c4)oc4ccccc45)nc(-c4c(-c5ccc6c(c5)sc5ccccc56)ccc5oc6ccccc6c45)n3)ccc2c1 ; c1ccc2c(c1)ccc1cc(-c3nc(-c4ccc5c(c4)oc4ccccc45)nc(-c4cccc5oc6c(-c7ccc8c(c7)sc7ccccc78)cccc6c45)n3)ccc12 ; c1ccc(-c2cccc(-c3nc(-c4ccc5c(c4)oc4ccccc45)nc(-c4cccc5oc6ccc(-c7cccc8c7sc7ccccc78)cc6c45)n3)c2)cc1]

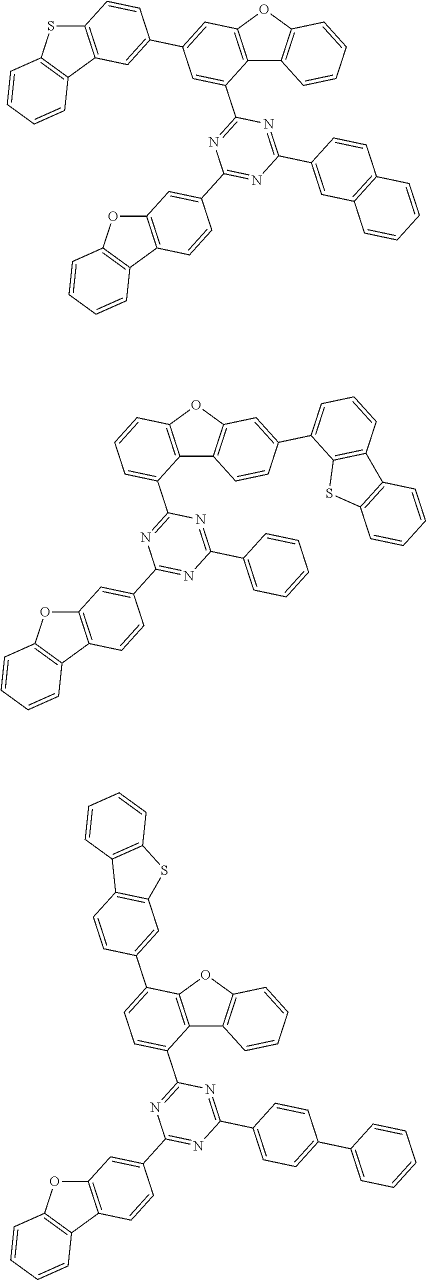 [SMILES: c1ccc2cc(-c3nc(-c4ccc5c(c4)oc4ccccc45)nc(-c4cc(-c5ccc6sc7ccccc7c6c5)cc5oc6ccccc6c45)n3)ccc2c1 ; c1ccc(-c2nc(-c3ccc4c(c3)oc3ccccc34)nc(-c3cccc4oc5cc(-c6cccc7c6sc6ccccc67)ccc5c34)n2)cc1 ; c1ccc(-c2ccc(-c3nc(-c4ccc5c(c4)oc4ccccc45)nc(-c4ccc(-c5ccc6c(c5)sc5ccccc56)c5oc6ccccc6c45)n3)cc2)cc1]

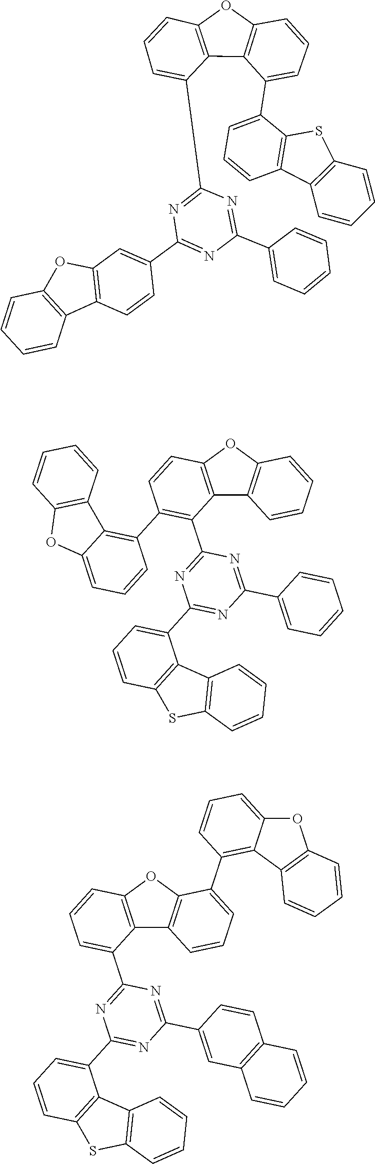 [SMILES: c1ccc(-c2nc(-c3ccc4c(c3)oc3ccccc34)nc(-c3cccc4oc5cccc(-c6cccc7c6sc6ccccc67)c5c34)n2)cc1 ; c1ccc(-c2nc(-c3c(-c4cccc5oc6ccccc6c45)ccc4oc5ccccc5c34)nc(-c3cccc4sc5ccccc5c34)n2)cc1 ; c1ccc2cc(-c3nc(-c4cccc5oc6c(-c7cccc8oc9ccccc9c78)cccc6c45)nc(-c4cccc5sc6ccccc6c45)n3)ccc2c1]

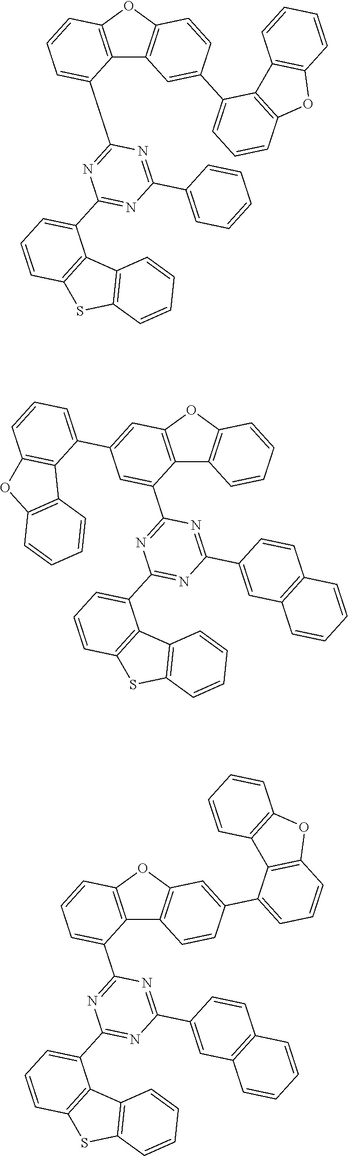 [SMILES: c1ccc(-c2nc(-c3cccc4oc5ccc(-c6cccc7oc8ccccc8c67)cc5c34)nc(-c3cccc4sc5ccccc5c34)n2)cc1 ; c1ccc2cc(-c3nc(-c4cc(-c5cccc6oc7ccccc7c56)cc5oc6ccccc6c45)nc(-c4cccc5sc6ccccc6c45)n3)ccc2c1 ; c1ccc2cc(-c3nc(-c4cccc5oc6cc(-c7cccc8oc9ccccc9c78)ccc6c45)nc(-c4cccc5sc6ccccc6c45)n3)ccc2c1]

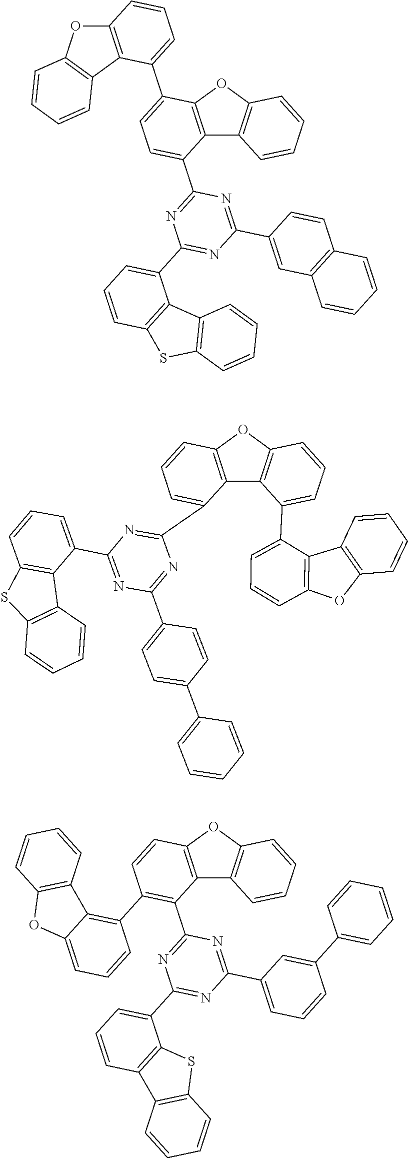 [SMILES: c1ccc2cc(-c3nc(-c4cccc5sc6ccccc6c45)nc(-c4ccc(-c5cccc6oc7ccccc7c56)c5oc6ccccc6c45)n3)ccc2c1 ; c1ccc(-c2ccc(-c3nc(-c4cccc5sc6ccccc6c45)nc(-c4cccc5oc6cccc(-c7cccc8oc9ccccc9c78)c6c45)n3)cc2)cc1 ; c1ccc(-c2cccc(-c3nc(-c4cccc5c4sc4ccccc45)nc(-c4c(-c5cccc6oc7ccccc7c56)ccc5oc6ccccc6c45)n3)c2)cc1]

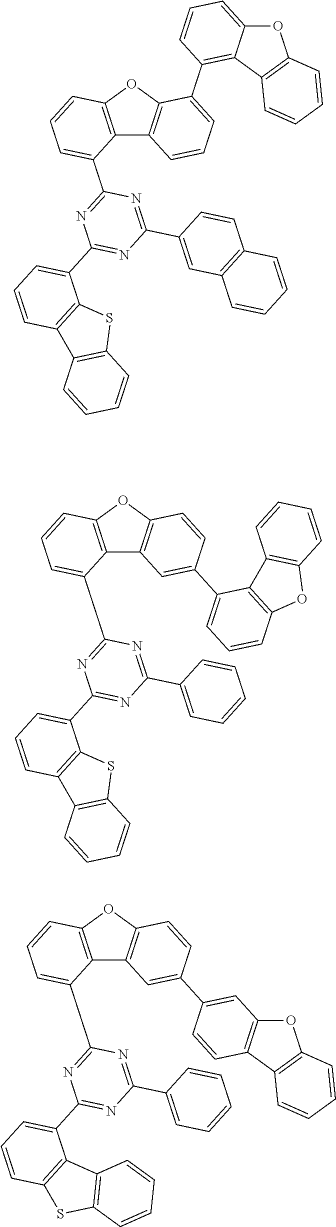 [SMILES: c1ccc2cc(-c3nc(-c4cccc5c4sc4ccccc45)nc(-c4cccc5oc6c(-c7cccc8oc9ccccc9c78)cccc6c45)n3)ccc2c1 ; c1ccc(-c2nc(-c3cccc4c3sc3ccccc34)nc(-c3cccc4oc5ccc(-c6cccc7oc8ccccc8c67)cc5c34)n2)cc1 ; c1ccc(-c2nc(-c3cccc4oc5ccc(-c6ccc7c(c6)oc6ccccc67)cc5c34)nc(-c3cccc4sc5ccccc5c34)n2)cc1]

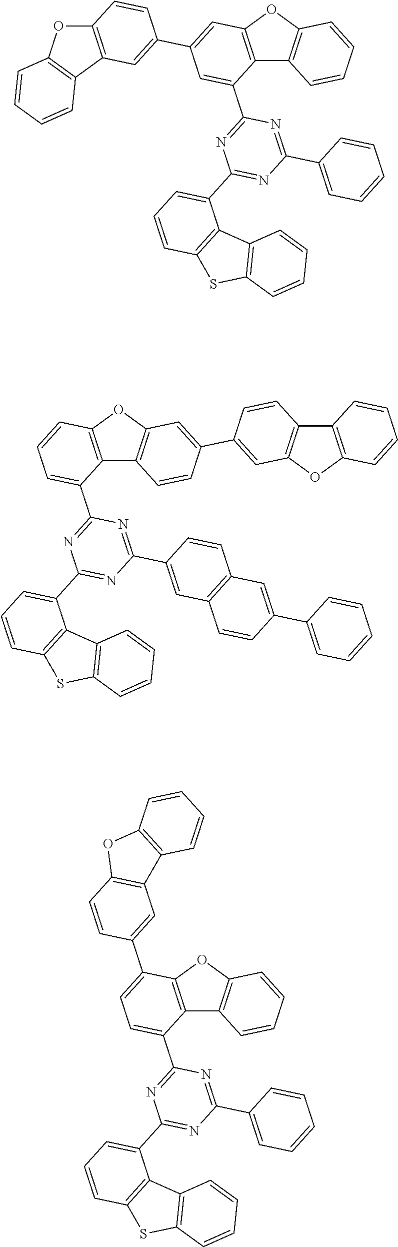 [SMILES: c1ccc(-c2nc(-c3cc(-c4ccc5oc6ccccc6c5c4)cc4oc5ccccc5c34)nc(-c3cccc4sc5ccccc5c34)n2)cc1 ; c1ccc(-c2ccc3cc(-c4nc(-c5cccc6oc7cc(-c8ccc9c(c8)oc8ccccc89)ccc7c56)nc(-c5cccc6sc7ccccc7c56)n4)ccc3c2)cc1 ; c1ccc(-c2nc(-c3cccc4sc5ccccc5c34)nc(-c3ccc(-c4ccc5oc6ccccc6c5c4)c4oc5ccccc5c34)n2)cc1]

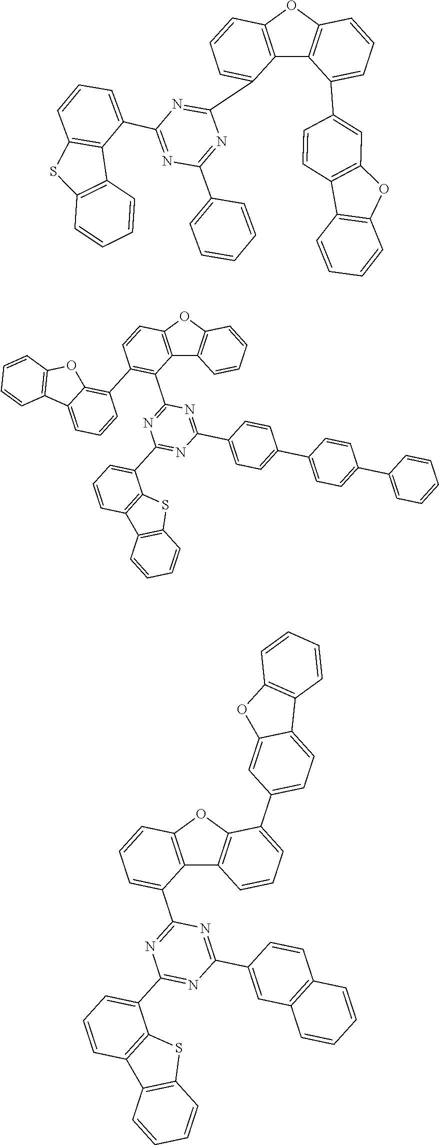 [SMILES: c1ccc(-c2nc(-c3cccc4sc5ccccc5c34)nc(-c3cccc4oc5cccc(-c6ccc7c(c6)oc6ccccc67)c5c34)n2)cc1 ; c1ccc(-c2ccc(-c3ccc(-c4nc(-c5cccc6c5sc5ccccc56)nc(-c5c(-c6cccc7c6oc6ccccc67)ccc6oc7ccccc7c56)n4)cc3)cc2)cc1 ; c1ccc2cc(-c3nc(-c4cccc5c4sc4ccccc45)nc(-c4cccc5oc6c(-c7ccc8c(c7)oc7ccccc78)cccc6c45)n3)ccc2c1]

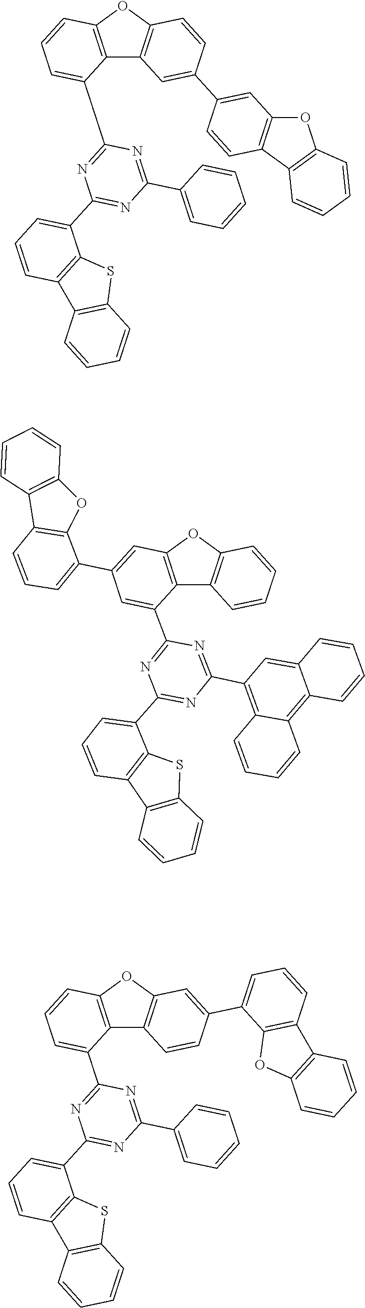 [SMILES: c1ccc(-c2nc(-c3cccc4c3sc3ccccc34)nc(-c3cccc4oc5ccc(-c6ccc7c(c6)oc6ccccc67)cc5c34)n2)cc1 ; c1ccc2c(c1)cc(-c1nc(-c3cccc4c3sc3ccccc34)nc(-c3cc(-c4cccc5c4oc4ccccc45)cc4oc5ccccc5c34)n1)c1ccccc12 ; c1ccc(-c2nc(-c3cccc4c3sc3ccccc34)nc(-c3cccc4oc5cc(-c6cccc7c6oc6ccccc67)ccc5c34)n2)cc1]

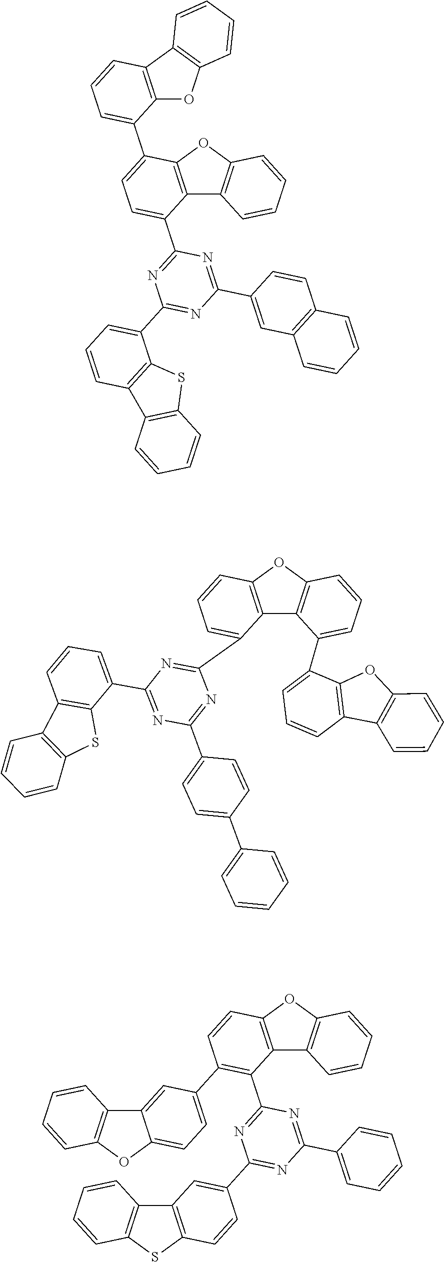 [SMILES: c1ccc2cc(-c3nc(-c4cccc5c4sc4ccccc45)nc(-c4ccc(-c5cccc6c5oc5ccccc56)c5oc6ccccc6c45)n3)ccc2c1 ; c1ccc(-c2ccc(-c3nc(-c4cccc5c4sc4ccccc45)nc(-c4cccc5oc6cccc(-c7cccc8c7oc7ccccc78)c6c45)n3)cc2)cc1 ; c1ccc(-c2nc(-c3ccc4sc5ccccc5c4c3)nc(-c3c(-c4ccc5oc6ccccc6c5c4)ccc4oc5ccccc5c34)n2)cc1]

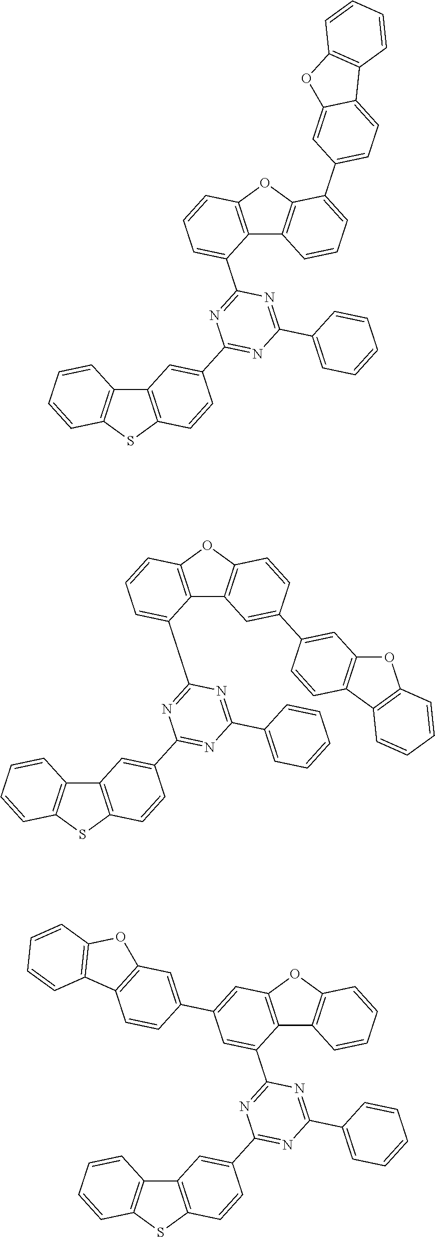 [SMILES: c1ccc(-c2nc(-c3ccc4sc5ccccc5c4c3)nc(-c3cccc4oc5c(-c6ccc7c(c6)oc6ccccc67)cccc5c34)n2)cc1 ; c1ccc(-c2nc(-c3ccc4sc5ccccc5c4c3)nc(-c3cccc4oc5ccc(-c6ccc7c(c6)oc6ccccc67)cc5c34)n2)cc1 ; c1ccc(-c2nc(-c3ccc4sc5ccccc5c4c3)nc(-c3cc(-c4ccc5c(c4)oc4ccccc45)cc4oc5ccccc5c34)n2)cc1]

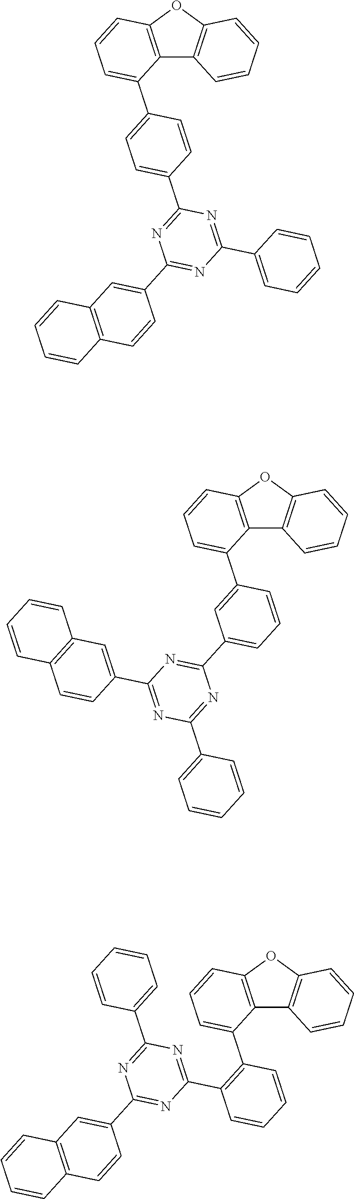 [SMILES: c1ccc(-c2nc(-c3ccc(-c4cccc5oc6ccccc6c45)cc3)nc(-c3ccc4ccccc4c3)n2)cc1 ; c1ccc(-c2nc(-c3cccc(-c4cccc5oc6ccccc6c45)c3)nc(-c3ccc4ccccc4c3)n2)cc1 ; c1ccc(-c2nc(-c3ccc4ccccc4c3)nc(-c3ccccc3-c3cccc4oc5ccccc5c34)n2)cc1]

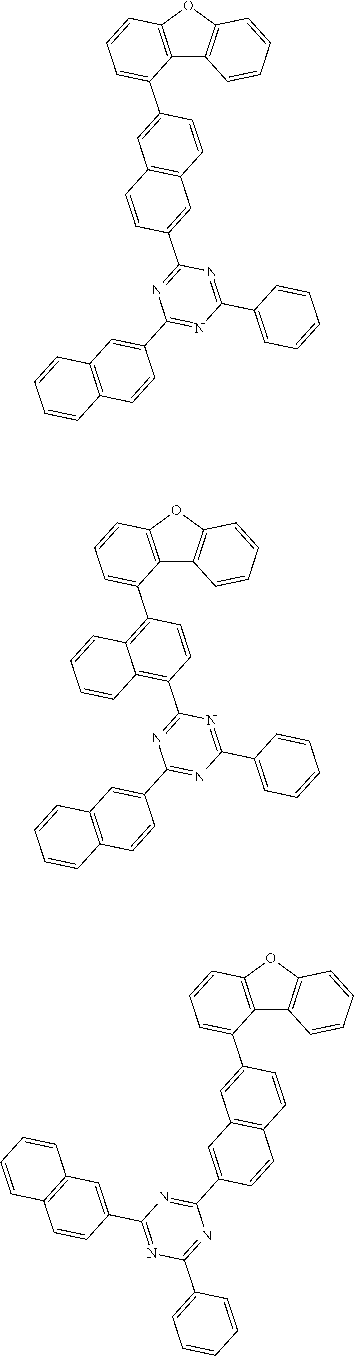 [SMILES: c1ccc(-c2nc(-c3ccc4ccccc4c3)nc(-c3ccc4cc(-c5cccc6oc7ccccc7c56)ccc4c3)n2)cc1 ; c1ccc(-c2nc(-c3ccc4ccccc4c3)nc(-c3ccc(-c4cccc5oc6ccccc6c45)c4ccccc34)n2)cc1 ; c1ccc(-c2nc(-c3ccc4ccccc4c3)nc(-c3ccc4ccc(-c5cccc6oc7ccccc7c56)cc4c3)n2)cc1]

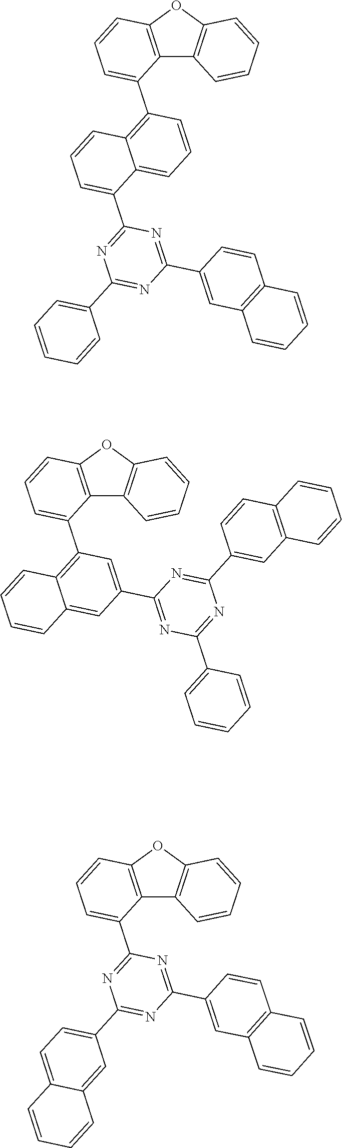 [SMILES: c1ccc(-c2nc(-c3ccc4ccccc4c3)nc(-c3cccc4c(-c5cccc6oc7ccccc7c56)cccc34)n2)cc1 ; c1ccc(-c2nc(-c3ccc4ccccc4c3)nc(-c3cc(-c4cccc5oc6ccccc6c45)c4ccccc4c3)n2)cc1 ; c1ccc2cc(-c3nc(-c4ccc5ccccc5c4)nc(-c4cccc5oc6ccccc6c45)n3)ccc2c1]

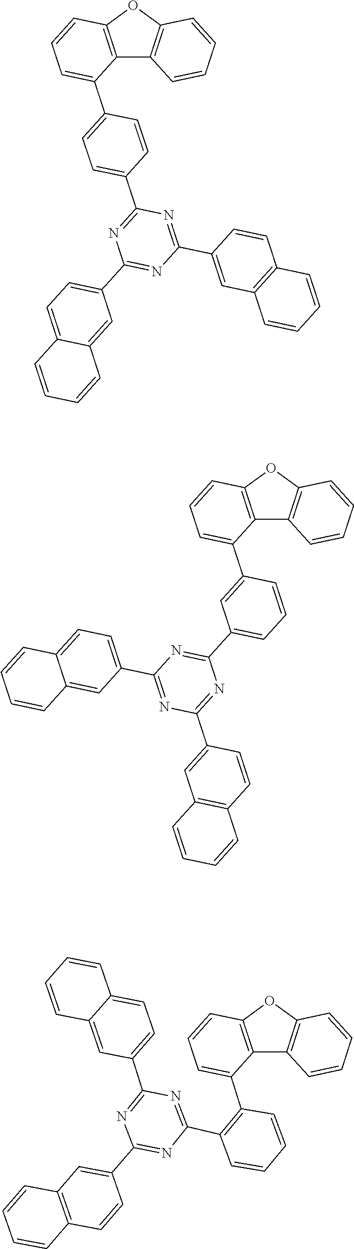 [SMILES: c1ccc2cc(-c3nc(-c4ccc(-c5cccc6oc7ccccc7c56)cc4)nc(-c4ccc5ccccc5c4)n3)ccc2c1 ; c1cc(-c2nc(-c3ccc4ccccc4c3)nc(-c3ccc4ccccc4c3)n2)cc(-c2cccc3oc4ccccc4c23)c1 ; c1ccc(-c2cccc3oc4ccccc4c23)c(-c2nc(-c3ccc4ccccc4c3)nc(-c3ccc4ccccc4c3)n2)c1]

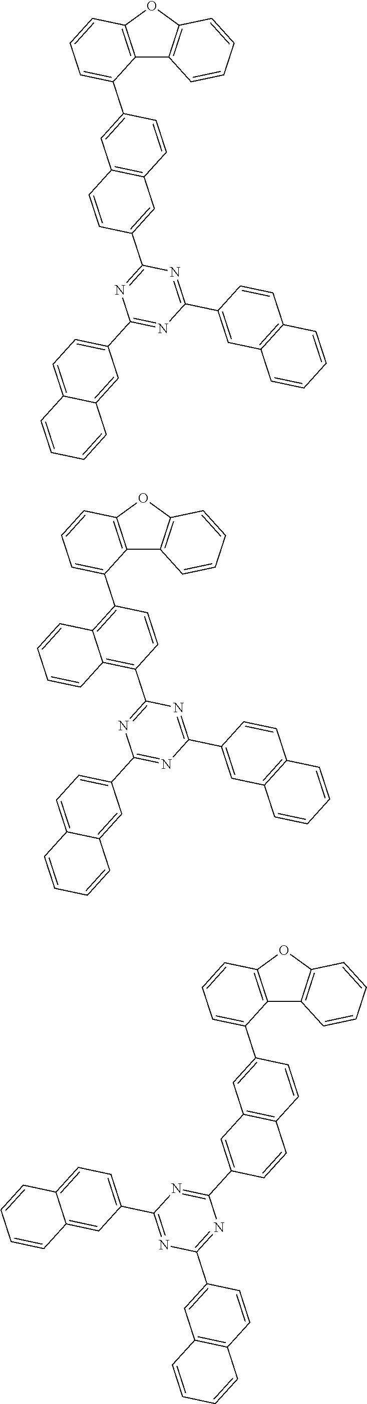 [SMILES: c1ccc2cc(-c3nc(-c4ccc5ccccc5c4)nc(-c4ccc5cc(-c6cccc7oc8ccccc8c67)ccc5c4)n3)ccc2c1 ; c1ccc2cc(-c3nc(-c4ccc5ccccc5c4)nc(-c4ccc(-c5cccc6oc7ccccc7c56)c5ccccc45)n3)ccc2c1 ; c1ccc2cc(-c3nc(-c4ccc5ccccc5c4)nc(-c4ccc5ccc(-c6cccc7oc8ccccc8c67)cc5c4)n3)ccc2c1]

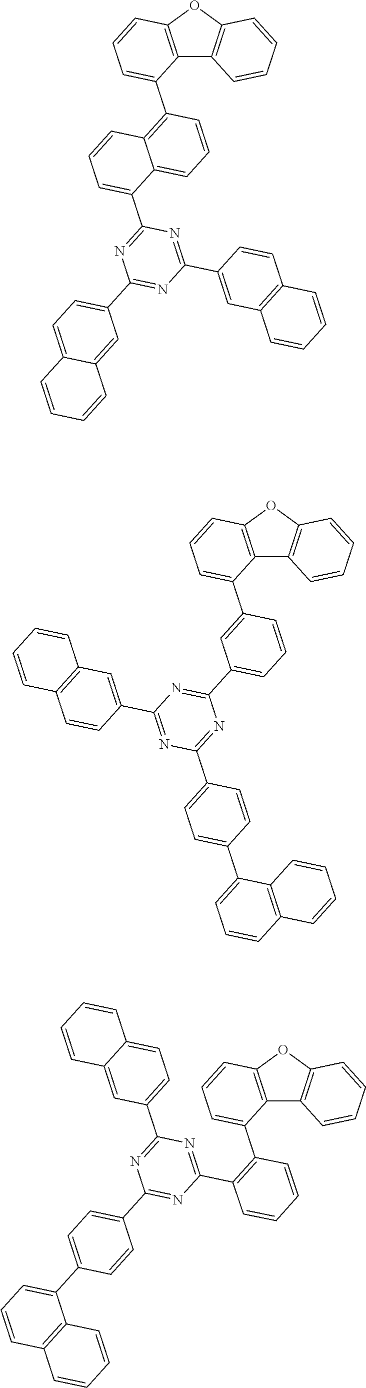 [SMILES: c1ccc2cc(-c3nc(-c4ccc5ccccc5c4)nc(-c4cccc5c(-c6cccc7oc8ccccc8c67)cccc45)n3)ccc2c1 ; c1cc(-c2nc(-c3ccc(-c4cccc5ccccc45)cc3)nc(-c3ccc4ccccc4c3)n2)cc(-c2cccc3oc4ccccc4c23)c1 ; c1ccc(-c2cccc3oc4ccccc4c23)c(-c2nc(-c3ccc(-c4cccc5ccccc45)cc3)nc(-c3ccc4ccccc4c3)n2)c1]

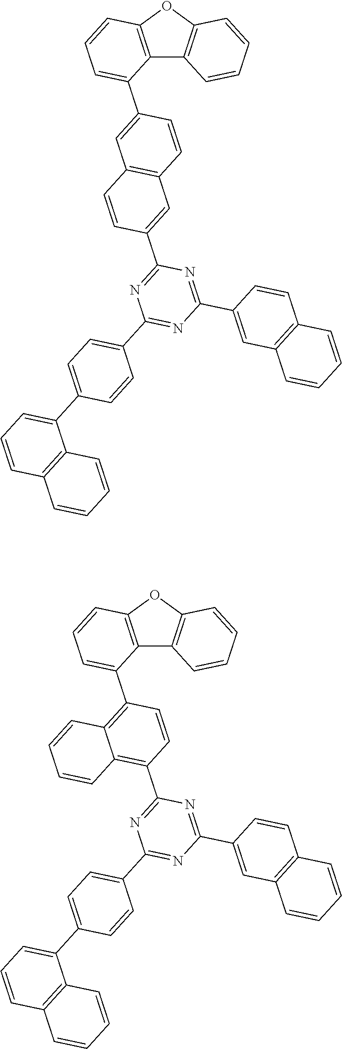 [SMILES: c1ccc2cc(-c3nc(-c4ccc(-c5cccc6ccccc56)cc4)nc(-c4ccc5cc(-c6cccc7oc8ccccc8c67)ccc5c4)n3)ccc2c1 ; c1ccc2cc(-c3nc(-c4ccc(-c5cccc6ccccc56)cc4)nc(-c4ccc(-c5cccc6oc7ccccc7c56)c5ccccc45)n3)ccc2c1]

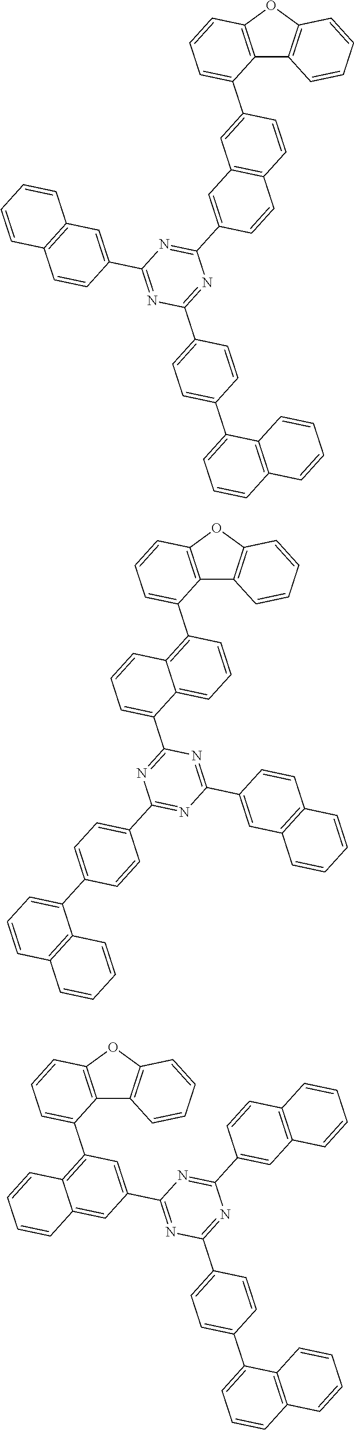

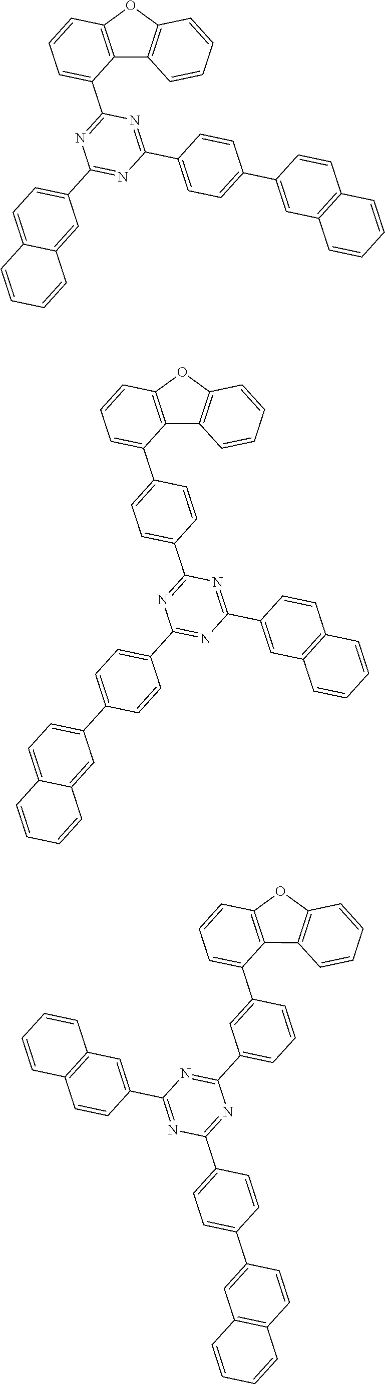 [SMILES: c1ccc2cc(-c3ccc(-c4nc(-c5ccc6ccccc6c5)nc(-c5cccc6oc7ccccc7c56)n4)cc3)ccc2c1 ; c1ccc2cc(-c3ccc(-c4nc(-c5ccc(-c6cccc7oc8ccccc8c67)cc5)nc(-c5ccc6ccccc6c5)n4)cc3)ccc2c1 ; c1cc(-c2nc(-c3ccc(-c4ccc5ccccc5c4)cc3)nc(-c3ccc4ccccc4c3)n2)cc(-c2cccc3oc4ccccc4c23)c1]

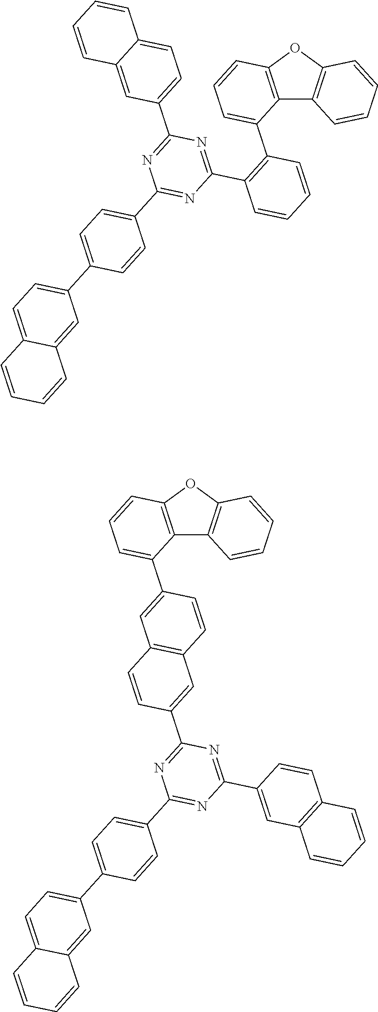 [SMILES: c1ccc(-c2cccc3oc4ccccc4c23)c(-c2nc(-c3ccc(-c4ccc5ccccc5c4)cc3)nc(-c3ccc4ccccc4c3)n2)c1 ; c1ccc2cc(-c3ccc(-c4nc(-c5ccc6ccccc6c5)nc(-c5ccc6cc(-c7cccc8oc9ccccc9c78)ccc6c5)n4)cc3)ccc2c1]

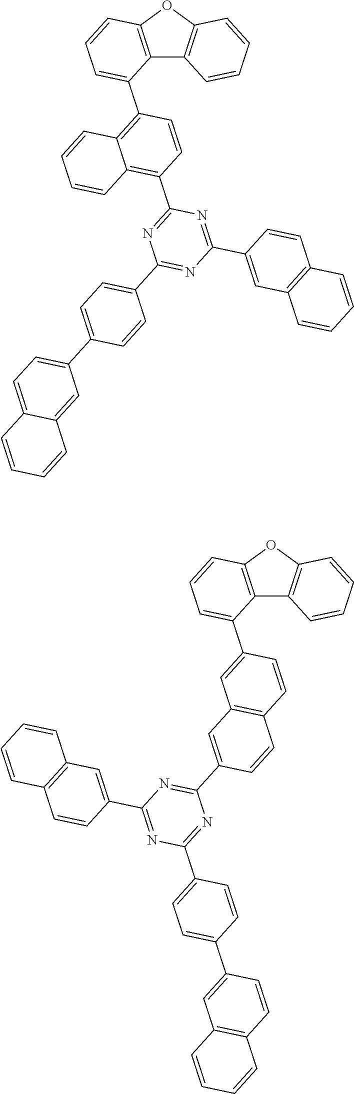 [SMILES: c1ccc2cc(-c3ccc(-c4nc(-c5ccc6ccccc6c5)nc(-c5ccc(-c6cccc7oc8ccccc8c67)c6ccccc56)n4)cc3)ccc2c1 ; c1ccc2cc(-c3ccc(-c4nc(-c5ccc6ccccc6c5)nc(-c5ccc6ccc(-c7cccc8oc9ccccc9c78)cc6c5)n4)cc3)ccc2c1]

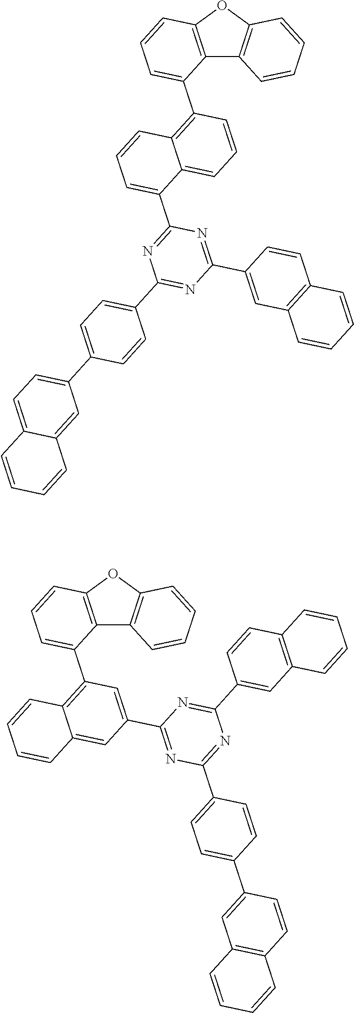 [SMILES: c1ccc2cc(-c3ccc(-c4nc(-c5ccc6ccccc6c5)nc(-c5cccc6c(-c7cccc8oc9ccccc9c78)cccc56)n4)cc3)ccc2c1 ; c1ccc2cc(-c3ccc(-c4nc(-c5ccc6ccccc6c5)nc(-c5cc(-c6cccc7oc8ccccc8c67)c6ccccc6c5)n4)cc3)ccc2c1]

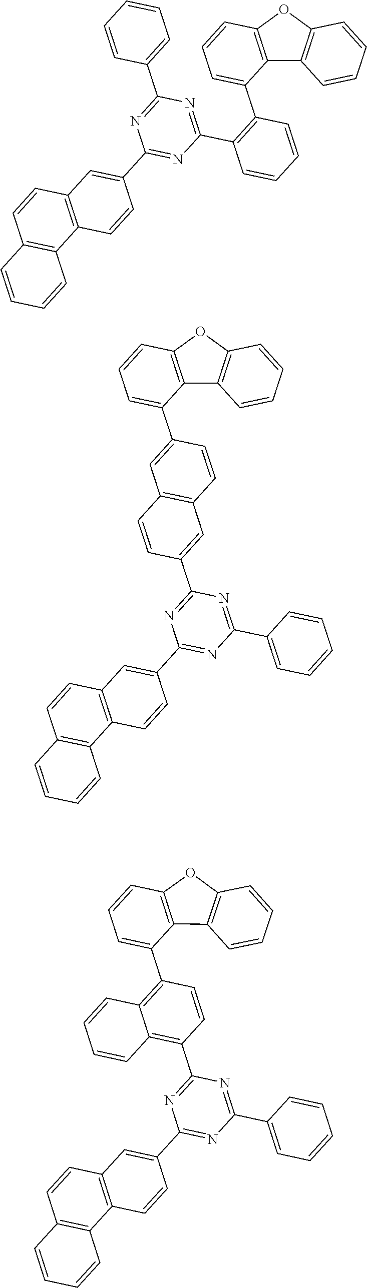 [SMILES: c1ccc(-c2nc(-c3ccc4c(ccc5ccccc54)c3)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4c(ccc5ccccc54)c3)n2)cc1 ; c1ccc(-c2nc(-c3ccc4c(ccc5ccccc54)c3)nc(-c3ccc(-c4cccc5oc6ccccc6c45)c4ccccc34)n2)cc1]

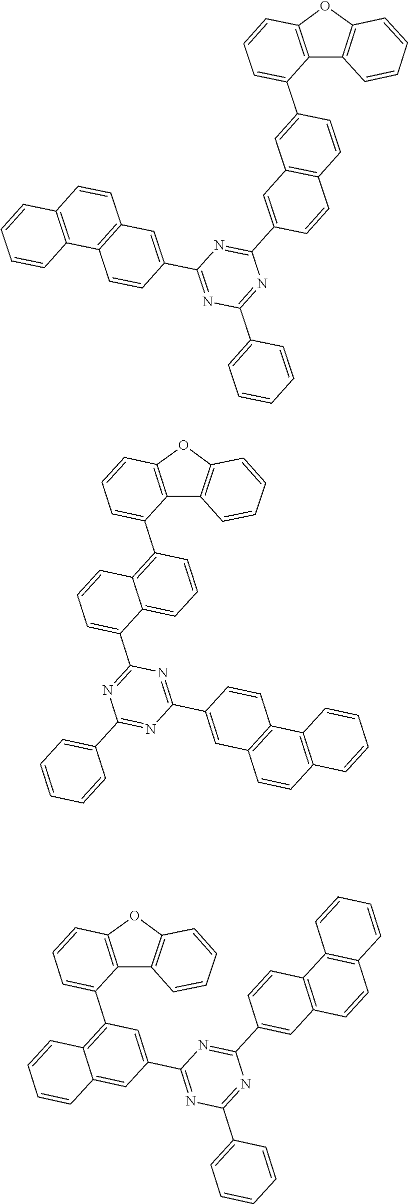 [SMILES: c1ccc(-c2nc(-c3ccc4ccc(-c5cccc6oc7ccccc7c56)cc4c3)nc(-c3ccc4c(ccc5ccccc54)c3)n2)cc1 ; c1ccc(-c2nc(-c3ccc4c(ccc5ccccc54)c3)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4c(ccc5ccccc54)c3)n2)cc1]

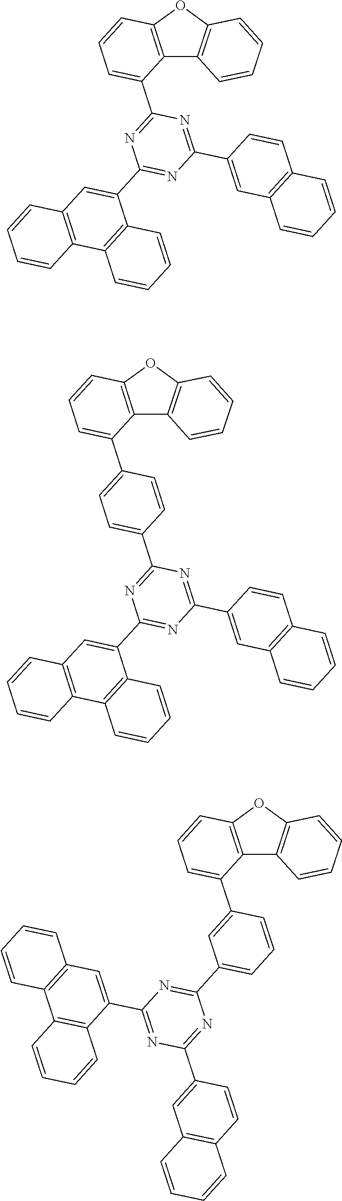 [SMILES: c1ccc2cc(-c3nc(-c4cc5ccccc5c5ccccc45)nc(-c4cccc5oc6ccccc6c45)n3)ccc2c1 ; c1ccc2cc(-c3nc(-c4ccc(-c5cccc6oc7ccccc7c56)cc4)nc(-c4cc5ccccc5c5ccccc45)n3)ccc2c1 ; c1cc(-c2nc(-c3ccc4ccccc4c3)nc(-c3cc4ccccc4c4ccccc34)n2)cc(-c2cccc3oc4ccccc4c23)c1]

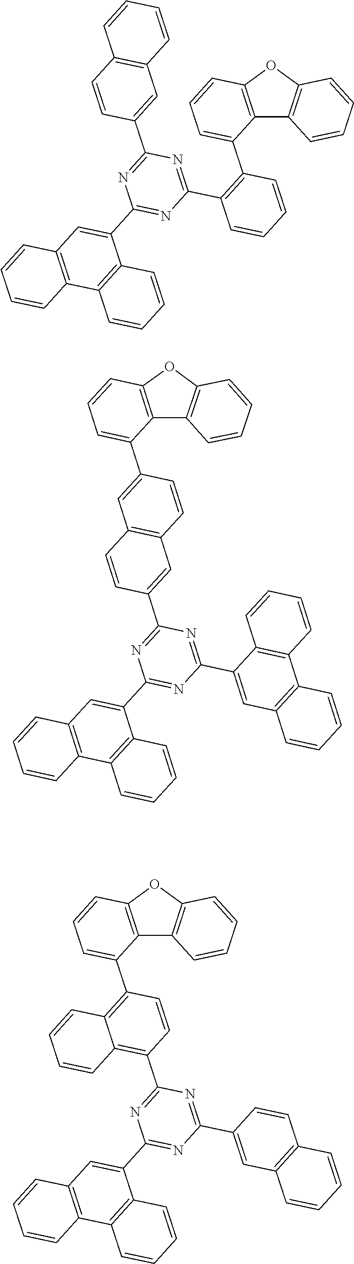 [SMILES: c1ccc(-c2cccc3oc4ccccc4c23)c(-c2nc(-c3ccc4ccccc4c3)nc(-c3cc4ccccc4c4ccccc34)n2)c1 ; c1ccc2c(c1)cc(-c1nc(-c3ccc4cc(-c5cccc6oc7ccccc7c56)ccc4c3)nc(-c3cc4ccccc4c4ccccc34)n1)c1ccccc12 ; c1ccc2cc(-c3nc(-c4ccc(-c5cccc6oc7ccccc7c56)c5ccccc45)nc(-c4cc5ccccc5c5ccccc45)n3)ccc2c1]

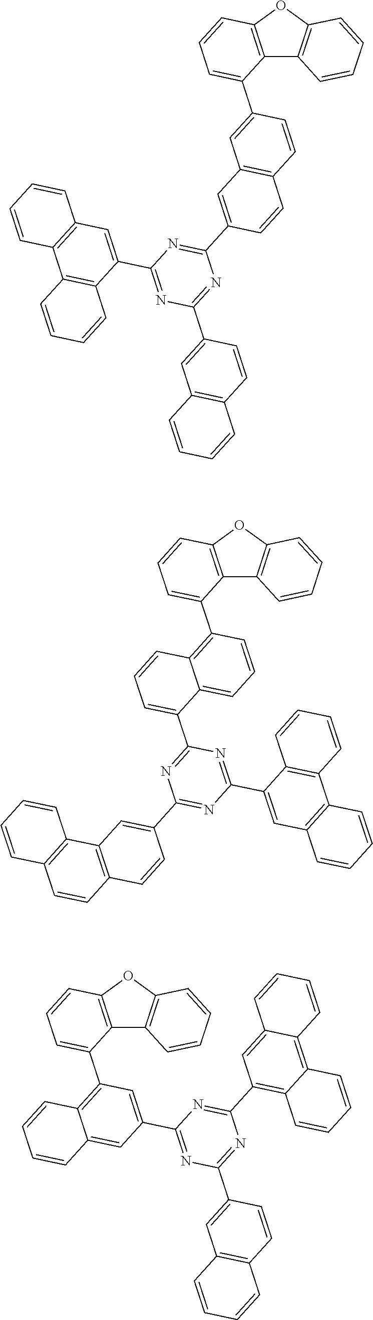 [SMILES: c1ccc2cc(-c3nc(-c4ccc5ccc(-c6cccc7oc8ccccc8c67)cc5c4)nc(-c4cc5ccccc5c5ccccc45)n3)ccc2c1 ; c1ccc2c(c1)ccc1ccc(-c3nc(-c4cccc5c(-c6cccc7oc8ccccc8c67)cccc45)nc(-c4cc5ccccc5c5ccccc45)n3)cc12 ; c1ccc2cc(-c3nc(-c4cc(-c5cccc6oc7ccccc7c56)c5ccccc5c4)nc(-c4cc5ccccc5c5ccccc45)n3)ccc2c1]

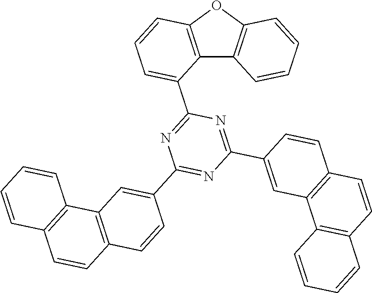 [SMILES: c1ccc2c(c1)ccc1ccc(-c3nc(-c4ccc5ccc6ccccc6c5c4)nc(-c4cccc5oc6ccccc6c45)n3)cc12]

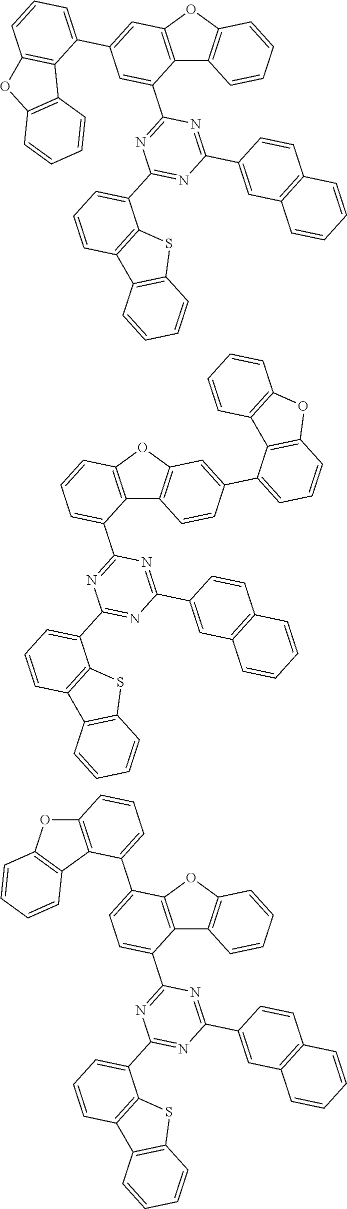

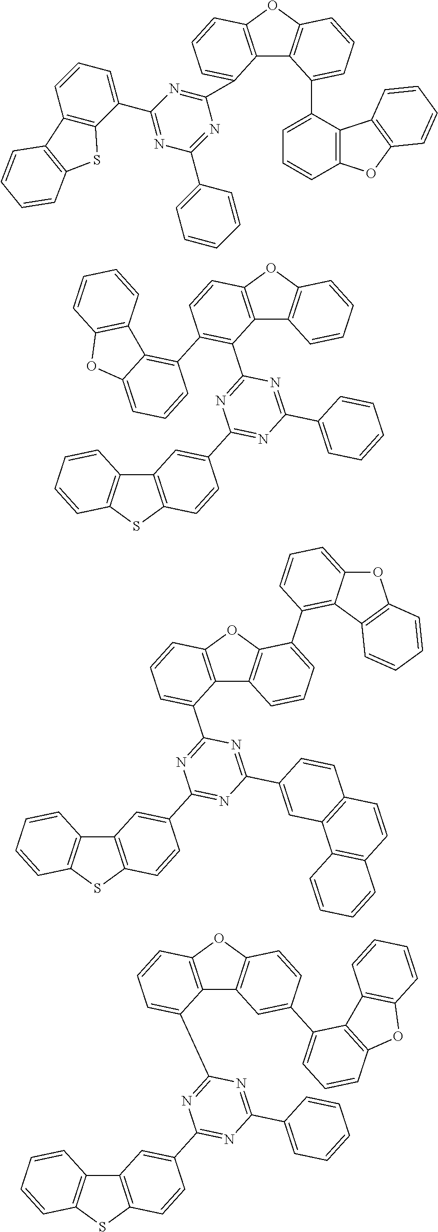

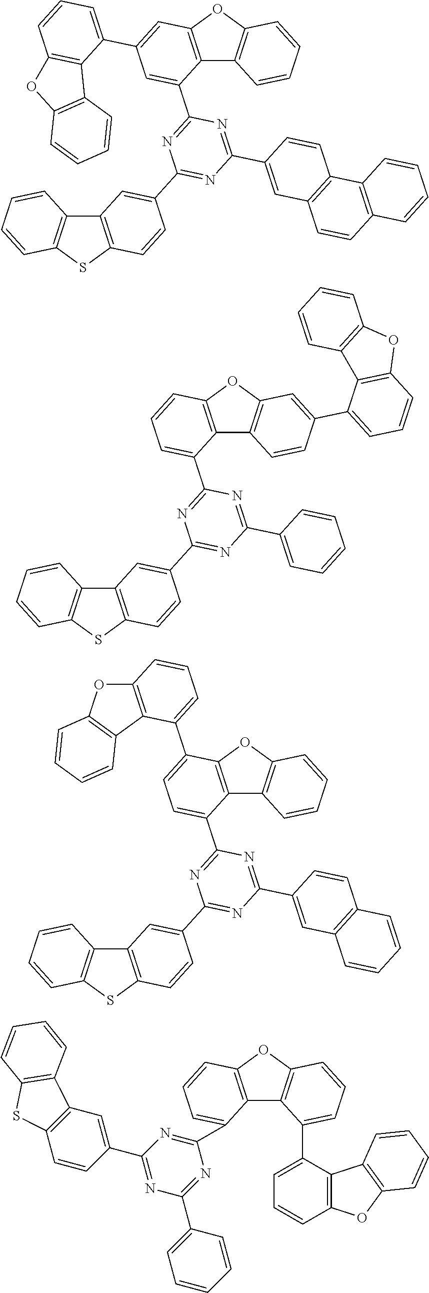

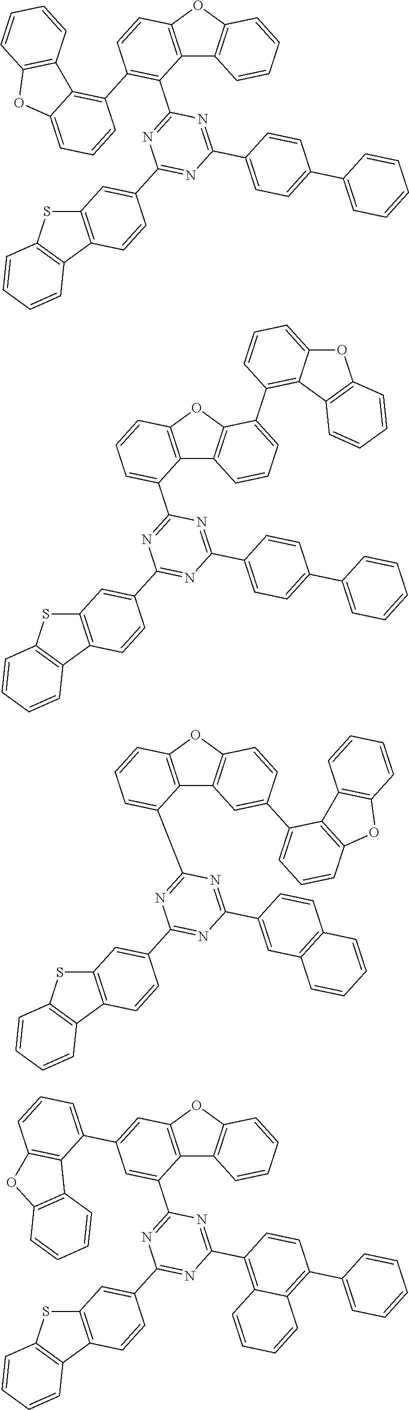

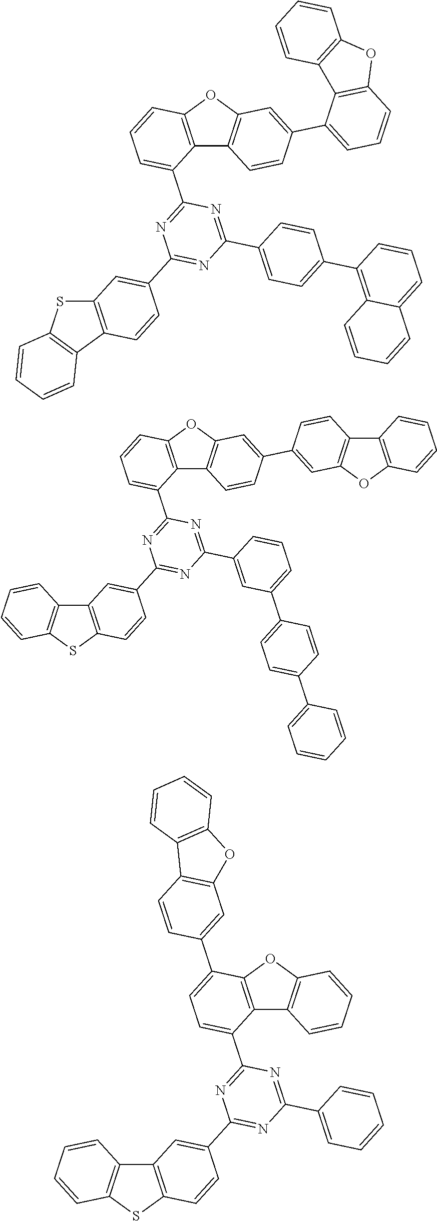 [SMILES: c1ccc2c(-c3ccc(-c4nc(-c5ccc6c(c5)sc5ccccc56)nc(-c5cccc6oc7cc(-c8cccc9oc%10ccccc%10c89)ccc7c56)n4)cc3)cccc2c1 ; c1ccc(-c2ccc(-c3cccc(-c4nc(-c5ccc6sc7ccccc7c6c5)nc(-c5cccc6oc7cc(-c8ccc9c(c8)oc8ccccc89)ccc7c56)n4)c3)cc2)cc1 ; c1ccc(-c2nc(-c3ccc4sc5ccccc5c4c3)nc(-c3ccc(-c4ccc5c(c4)oc4ccccc45)c4oc5ccccc5c34)n2)cc1]

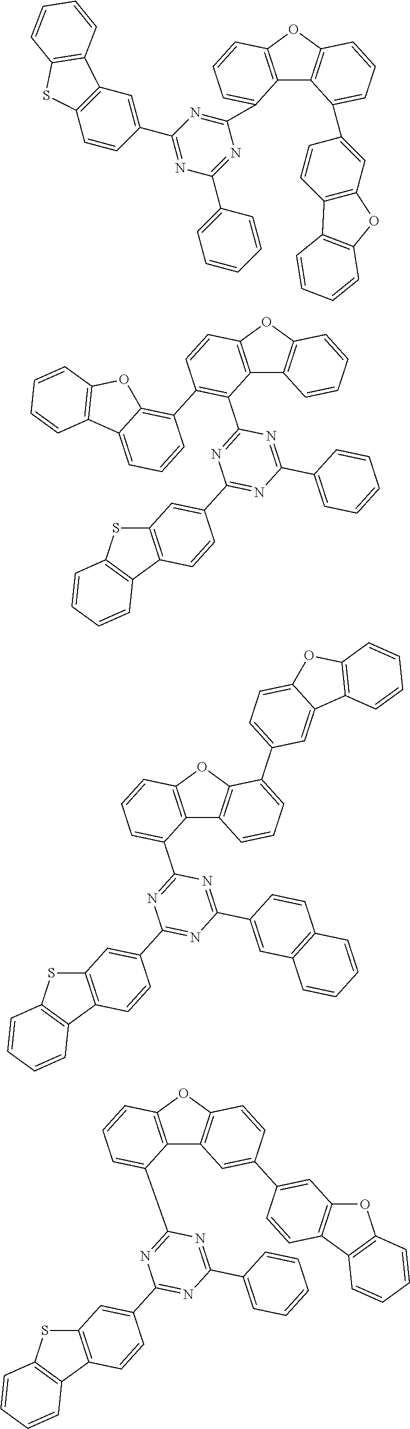

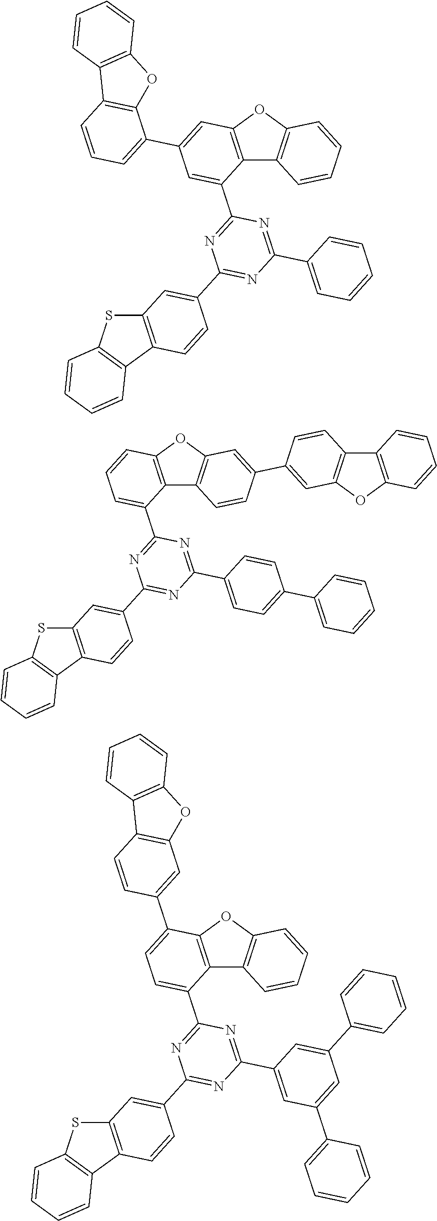 [SMILES: c1ccc(-c2nc(-c3ccc4c(c3)sc3ccccc34)nc(-c3cc(-c4cccc5c4oc4ccccc45)cc4oc5ccccc5c34)n2)cc1 ; c1ccc(-c2ccc(-c3nc(-c4ccc5c(c4)sc4ccccc45)nc(-c4cccc5oc6cc(-c7ccc8c(c7)oc7ccccc78)ccc6c45)n3)cc2)cc1 ; c1ccc(-c2cc(-c3ccccc3)cc(-c3nc(-c4ccc5c(c4)sc4ccccc45)nc(-c4ccc(-c5ccc6c(c5)oc5ccccc56)c5oc6ccccc6c45)n3)c2)cc1]

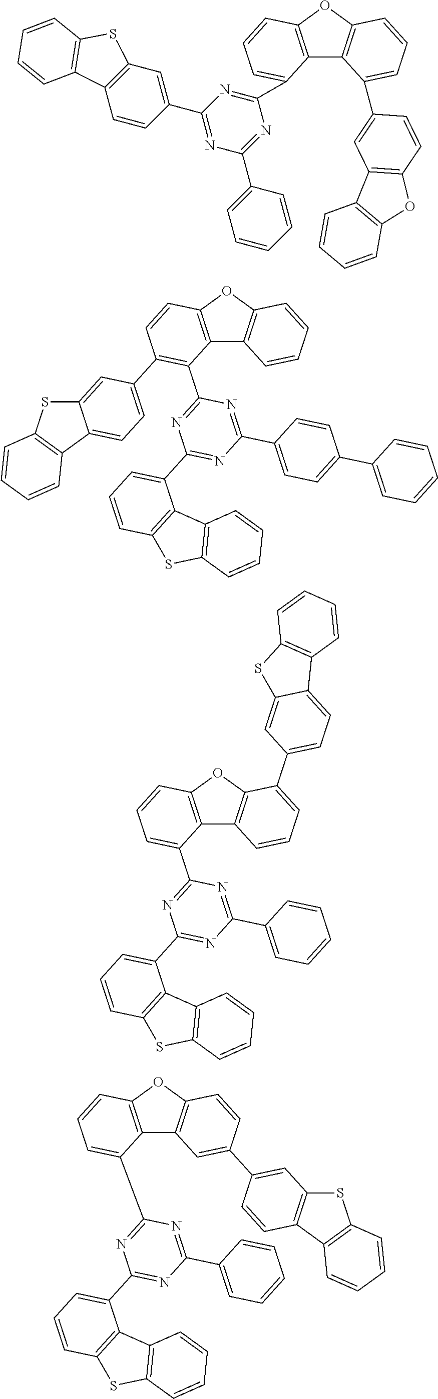

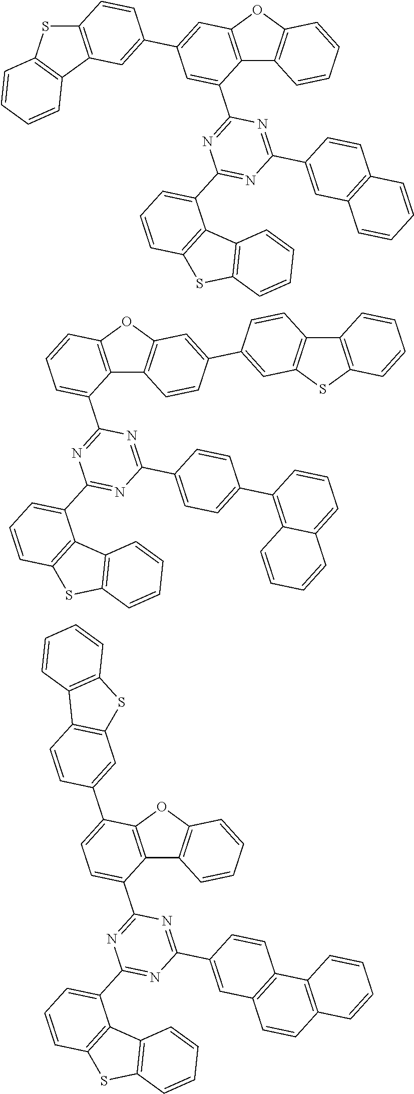

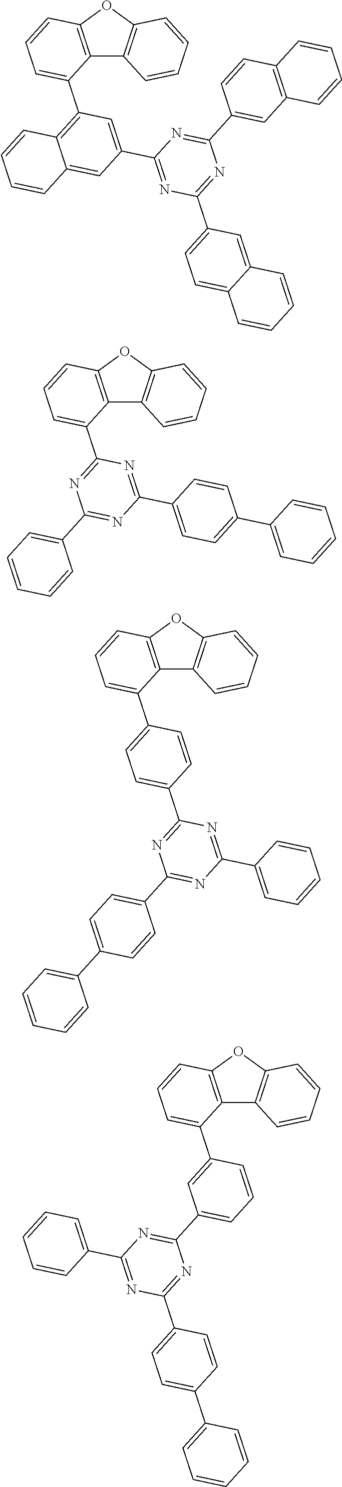 [SMILES: c1ccc2cc(-c3nc(-c4ccc5ccccc5c4)nc(-c4cc(-c5cccc6oc7ccccc7c56)c5ccccc5c4)n3)ccc2c1 ; c1ccc(-c2ccc(-c3nc(-c4ccccc4)nc(-c4cccc5oc6ccccc6c45)n3)cc2)cc1 ; c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4cccc(-c5cccc6oc7ccccc7c56)c4)n3)cc2)cc1]

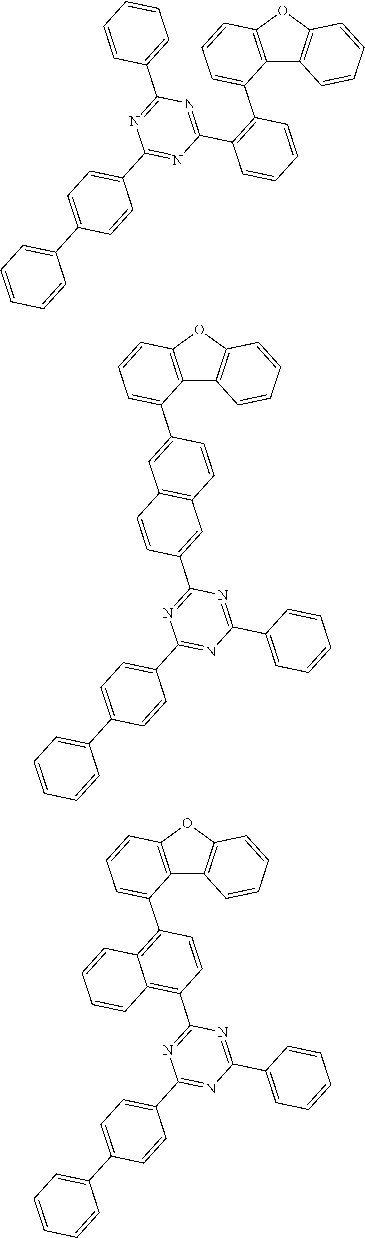 [SMILES: c1ccc(-c2ccc(-c3nc(-c4ccccc4)nc(-c4ccccc4-c4cccc5oc6ccccc6c45)n3)cc2)cc1 ; c1ccc(-c2ccc(-c3nc(-c4ccccc4)nc(-c4ccc5cc(-c6cccc7oc8ccccc8c67)ccc5c4)n3)cc2)cc1 ; c1ccc(-c2ccc(-c3nc(-c4ccccc4)nc(-c4ccc(-c5cccc6oc7ccccc7c56)c5ccccc45)n3)cc2)cc1]

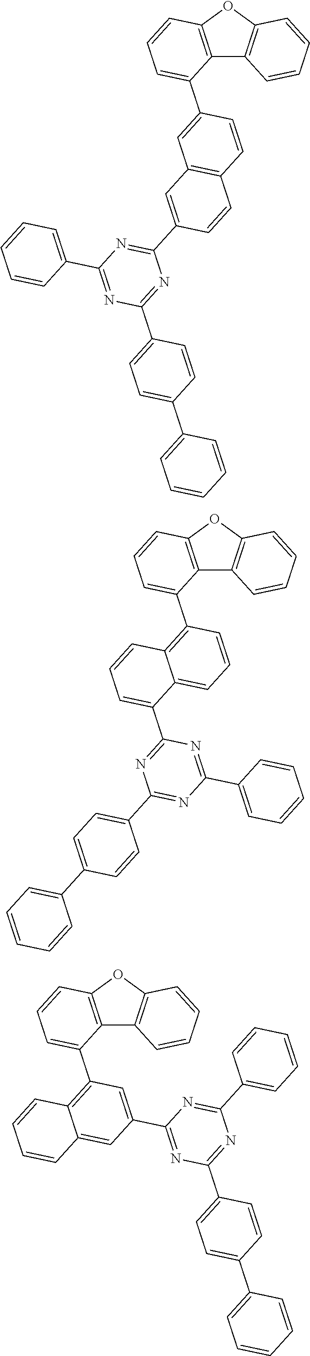

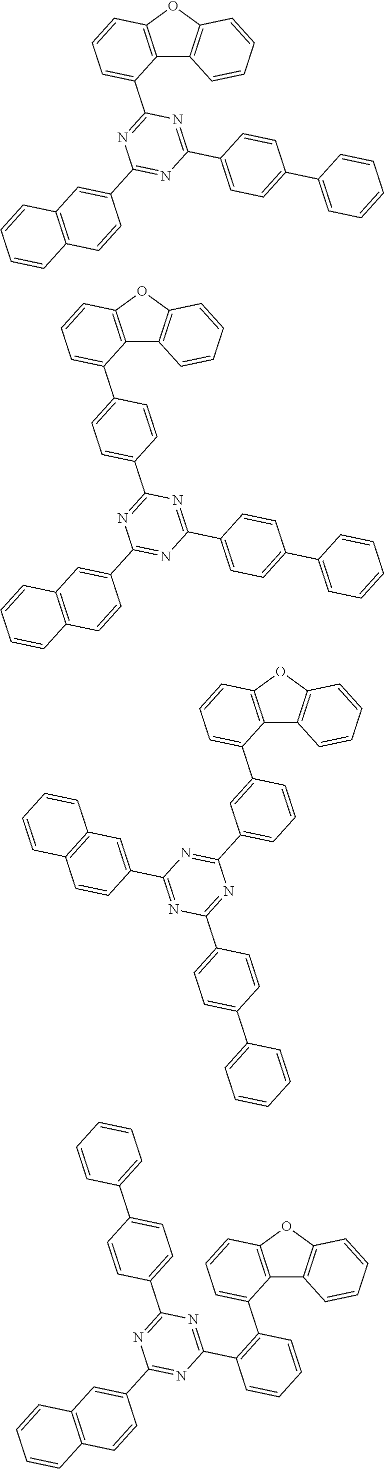 [SMILES: c1ccc(-c2ccc(-c3nc(-c4ccc5ccccc5c4)nc(-c4cccc5oc6ccccc6c45)n3)cc2)cc1 ; c1ccc(-c2ccc(-c3nc(-c4ccc(-c5cccc6oc7ccccc7c56)cc4)nc(-c4ccc5ccccc5c4)n3)cc2)cc1 ; c1ccc(-c2ccc(-c3nc(-c4cccc(-c5cccc6oc7ccccc7c56)c4)nc(-c4ccc5ccccc5c4)n3)cc2)cc1 ; c1ccc(-c2ccc(-c3nc(-c4ccc5ccccc5c4)nc(-c4ccccc4-c4cccc5oc6ccccc6c45)n3)cc2)cc1]

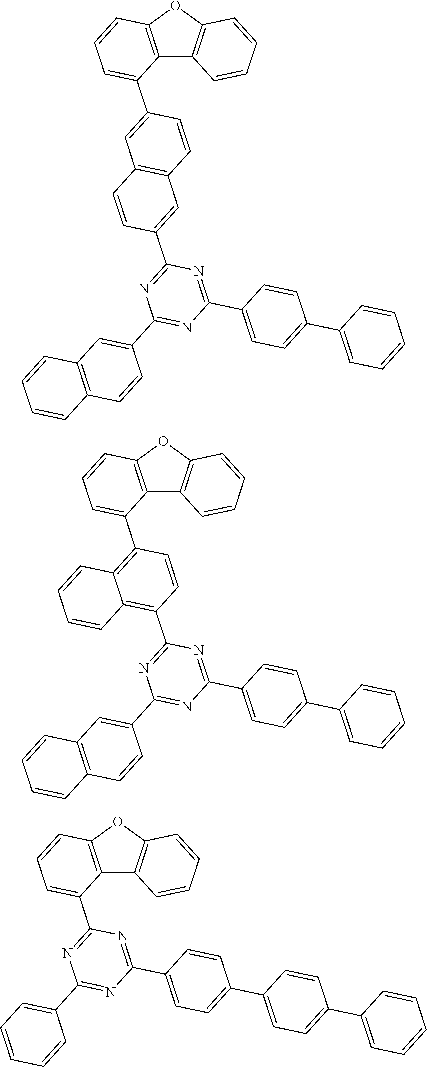

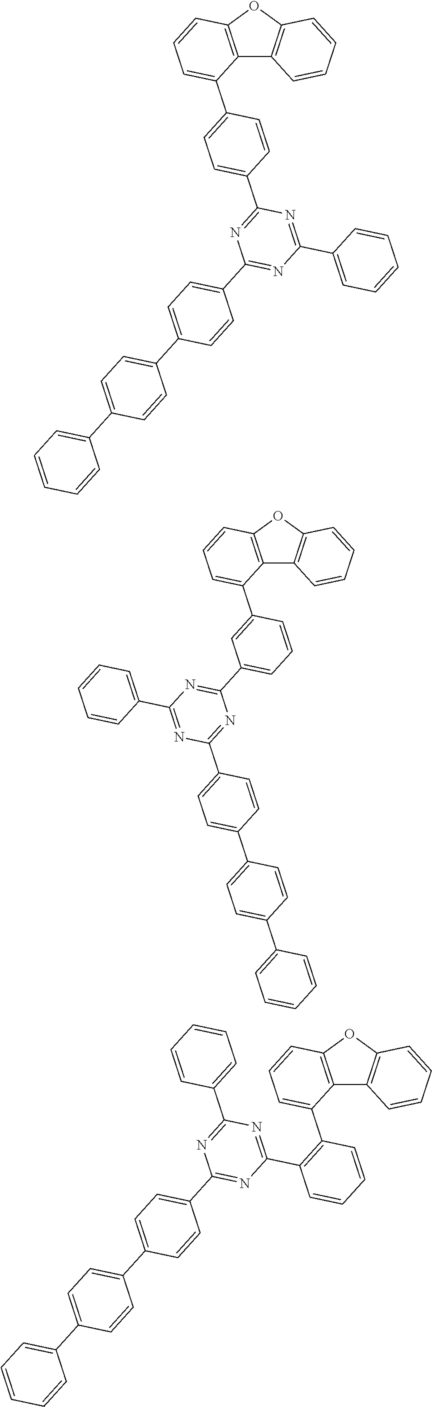

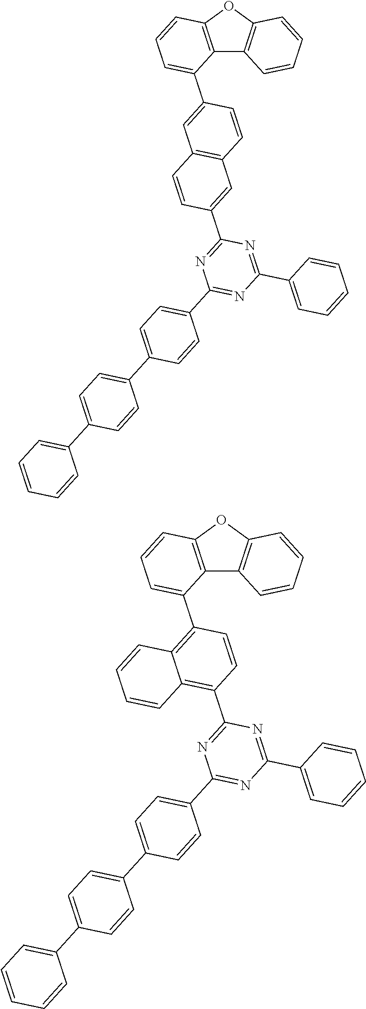 [SMILES: c1ccc(-c2ccc(-c3ccc(-c4nc(-c5ccccc5)nc(-c5ccc6cc(-c7cccc8oc9ccccc9c78)ccc6c5)n4)cc3)cc2)cc1 ; c1ccc(-c2ccc(-c3ccc(-c4nc(-c5ccccc5)nc(-c5ccc(-c6cccc7oc8ccccc8c67)c6ccccc56)n4)cc3)cc2)cc1]

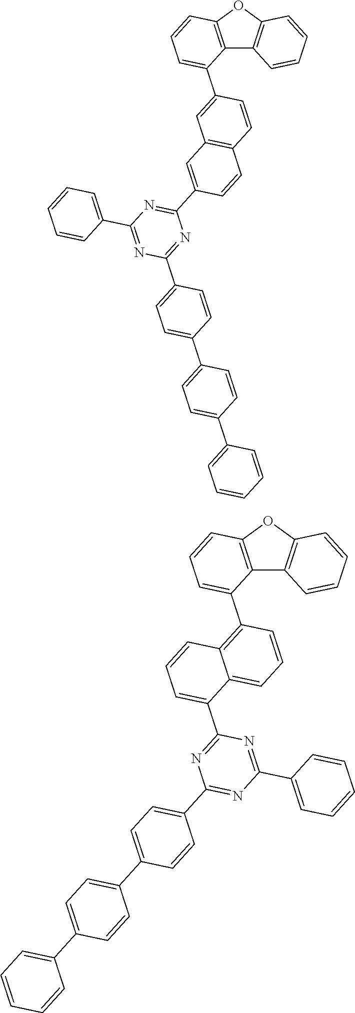

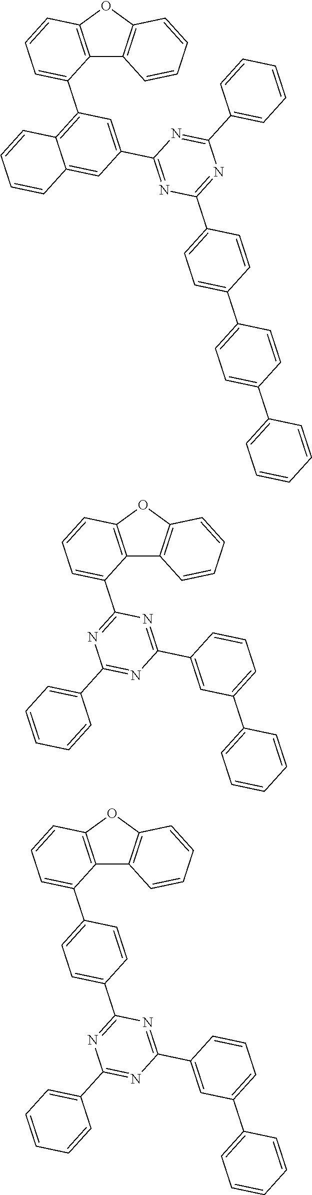 [SMILES: c1ccc(-c2ccc(-c3ccc(-c4nc(-c5ccccc5)nc(-c5cc(-c6cccc7oc8ccccc8c67)c6ccccc6c5)n4)cc3)cc2)cc1 ; c1ccc(-c2cccc(-c3nc(-c4ccccc4)nc(-c4cccc5oc6ccccc6c45)n3)c2)cc1 ; c1ccc(-c2cccc(-c3nc(-c4ccccc4)nc(-c4ccc(-c5cccc6oc7ccccc7c56)cc4)n3)c2)cc1]

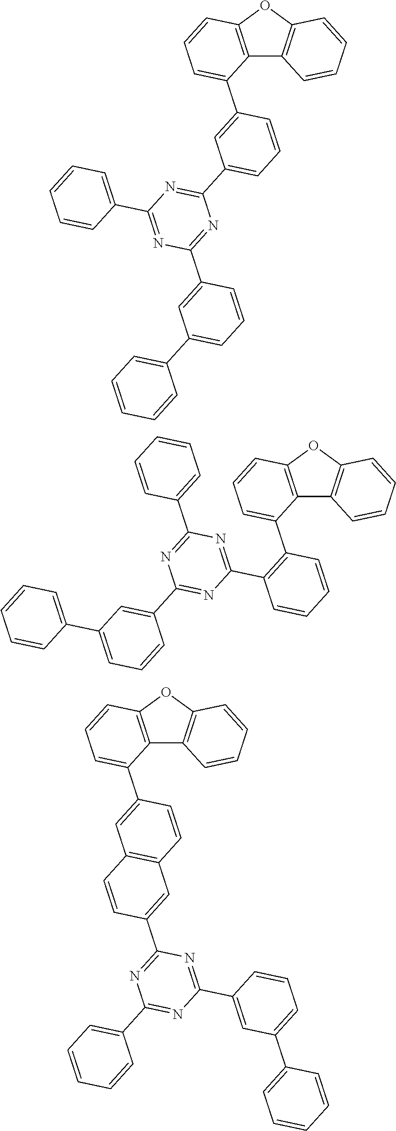

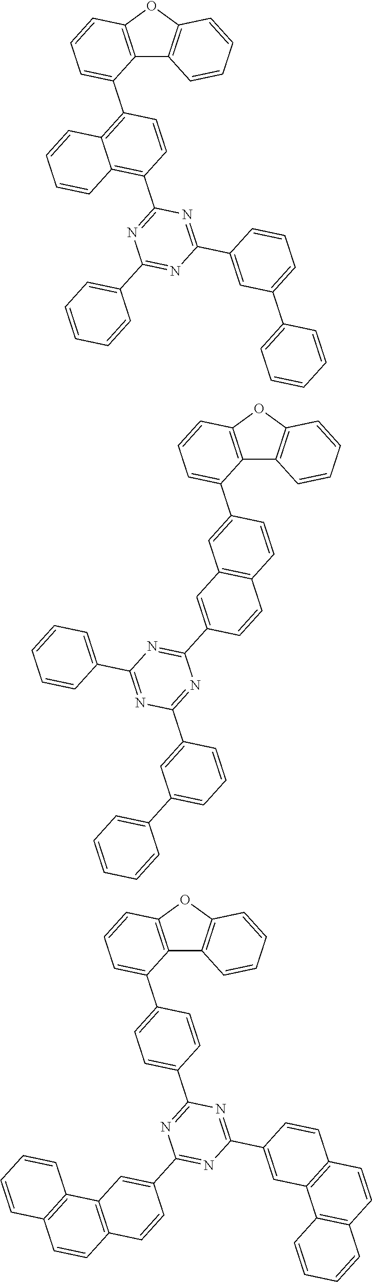

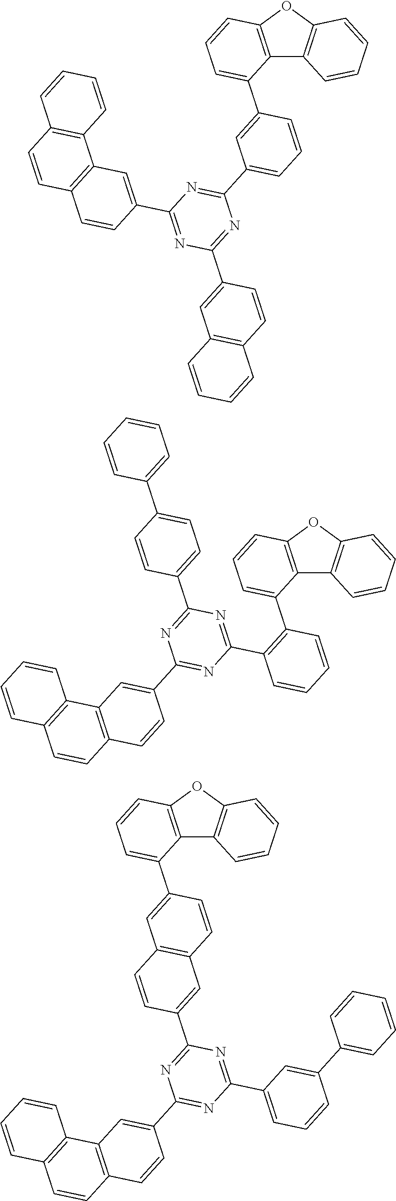 [SMILES: c1cc(-c2nc(-c3ccc4ccccc4c3)nc(-c3ccc4ccc5ccccc5c4c3)n2)cc(-c2cccc3oc4ccccc4c23)c1 ; c1ccc(-c2ccc(-c3nc(-c4ccc5ccc6ccccc6c5c4)nc(-c4ccccc4-c4cccc5oc6ccccc6c45)n3)cc2)cc1 ; c1ccc(-c2cccc(-c3nc(-c4ccc5cc(-c6cccc7oc8ccccc8c67)ccc5c4)nc(-c4ccc5ccc6ccccc6c5c4)n3)c2)cc1]

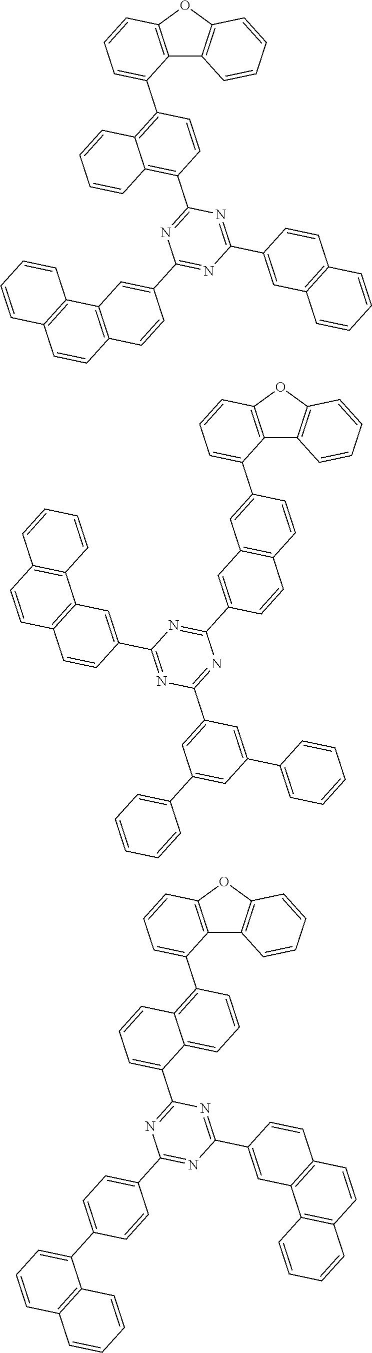

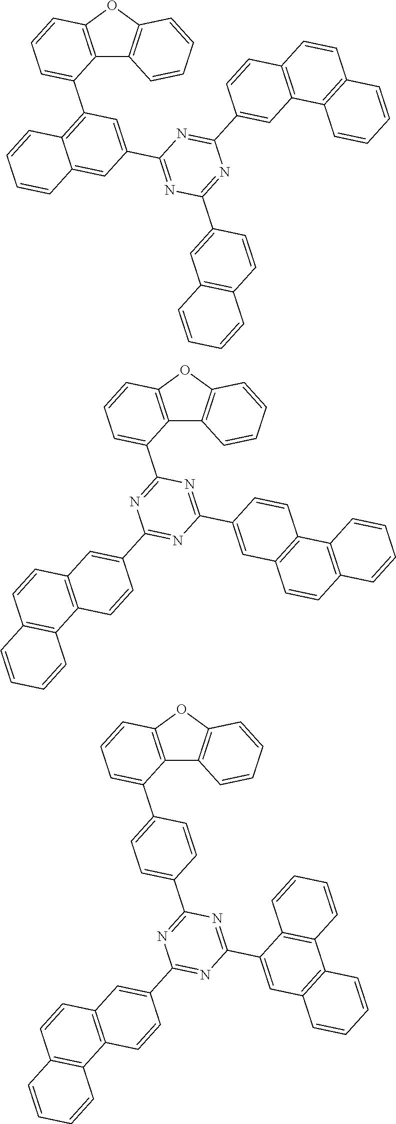

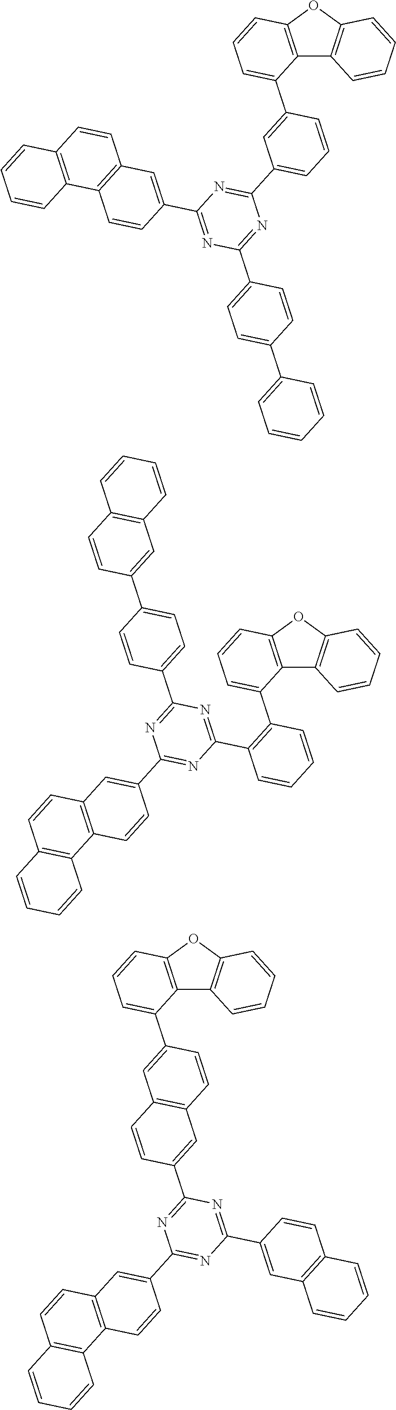 [SMILES: c1ccc(-c2ccc(-c3nc(-c4cccc(-c5cccc6oc7ccccc7c56)c4)nc(-c4ccc5c(ccc6ccccc65)c4)n3)cc2)cc1 ; c1ccc(-c2cccc3oc4ccccc4c23)c(-c2nc(-c3ccc(-c4ccc5ccccc5c4)cc3)nc(-c3ccc4c(ccc5ccccc54)c3)n2)c1 ; c1ccc2cc(-c3nc(-c4ccc5cc(-c6cccc7oc8ccccc8c67)ccc5c4)nc(-c4ccc5c(ccc6ccccc65)c4)n3)ccc2c1]

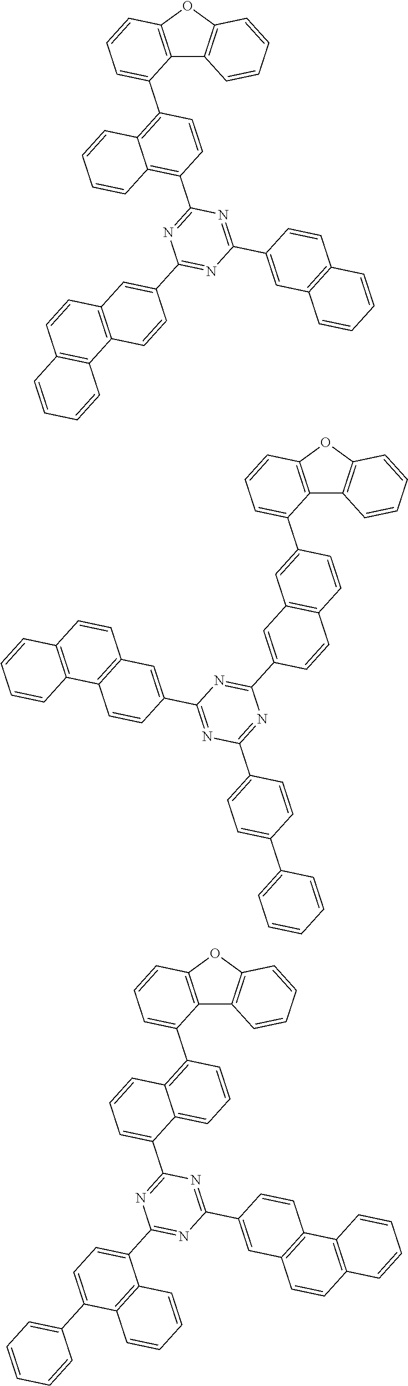

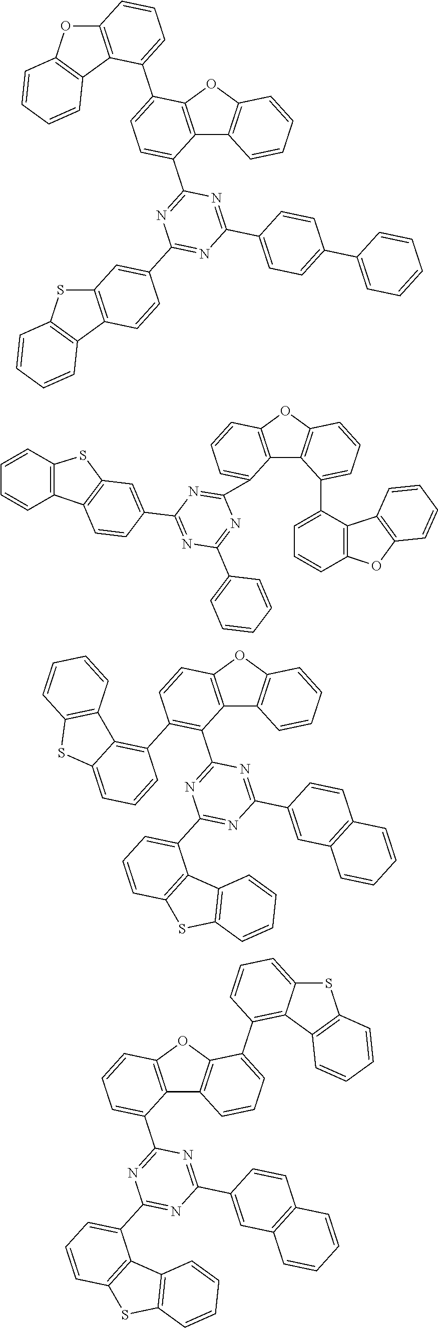

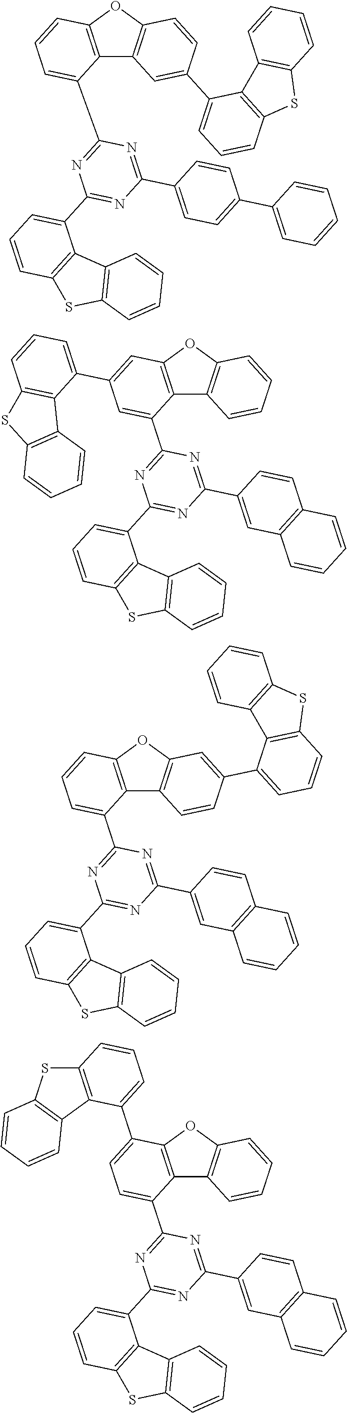

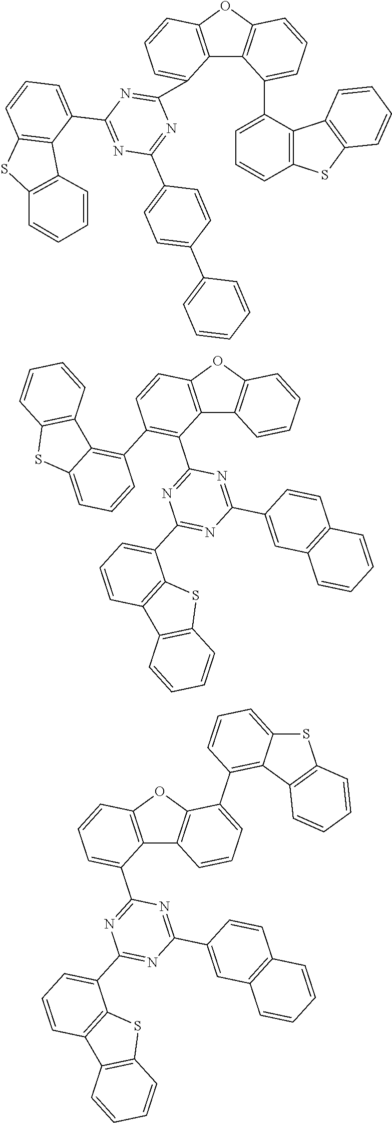

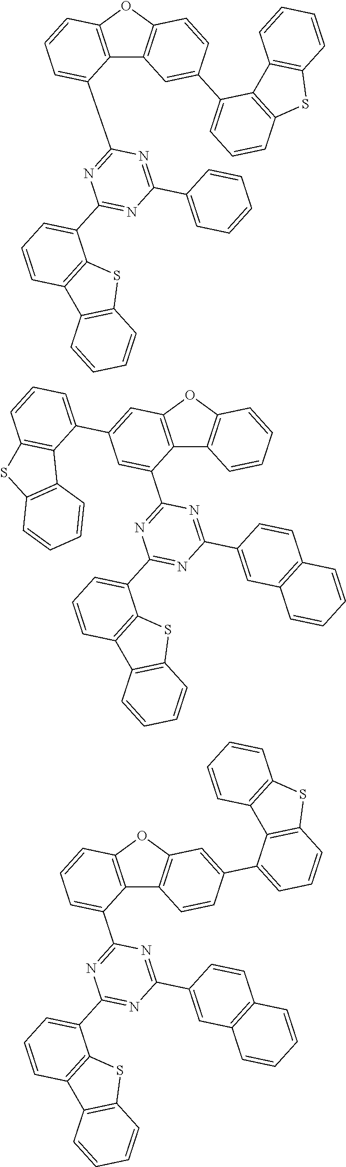

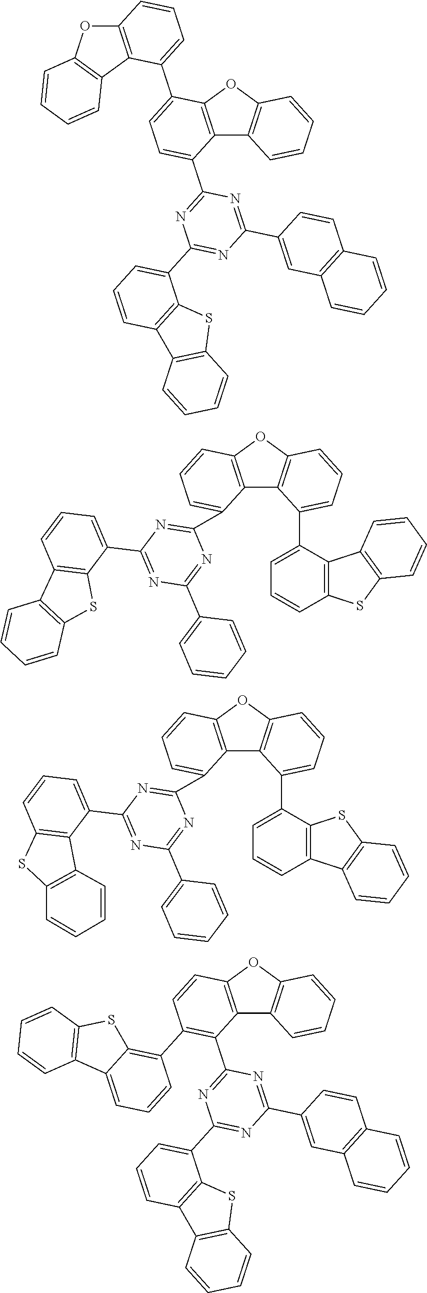

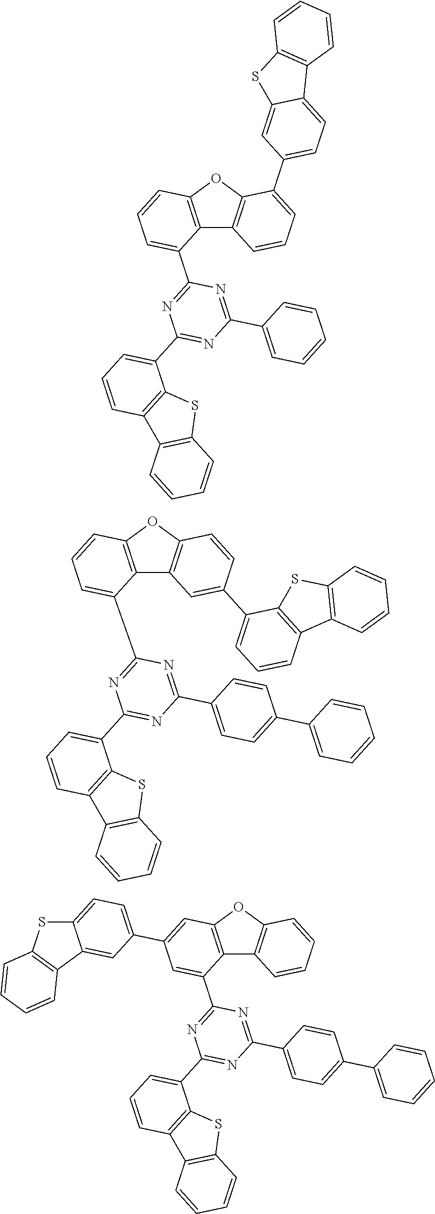

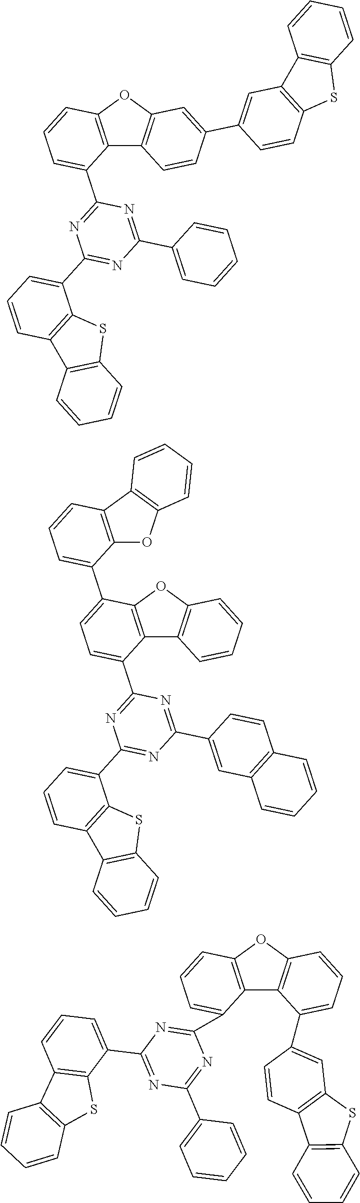

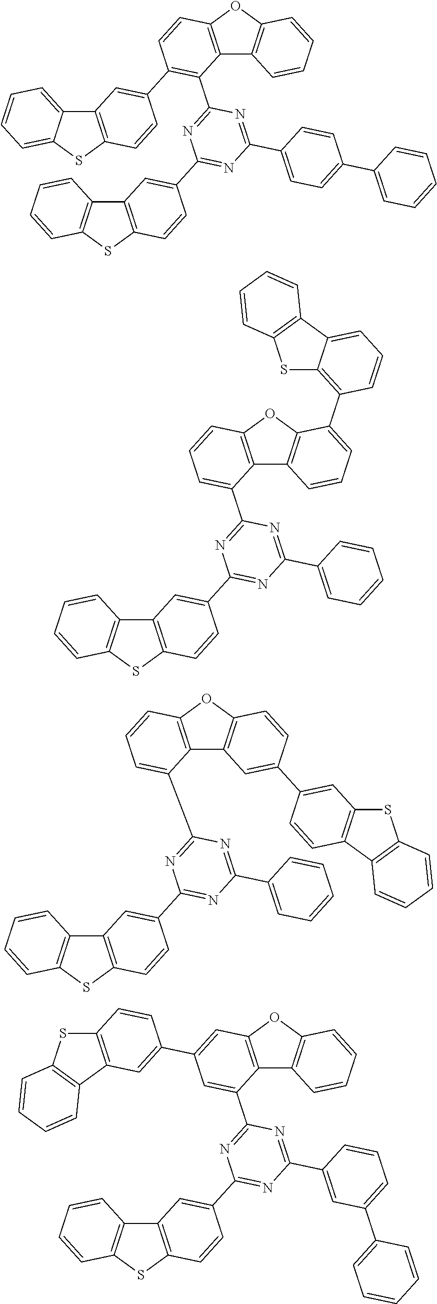

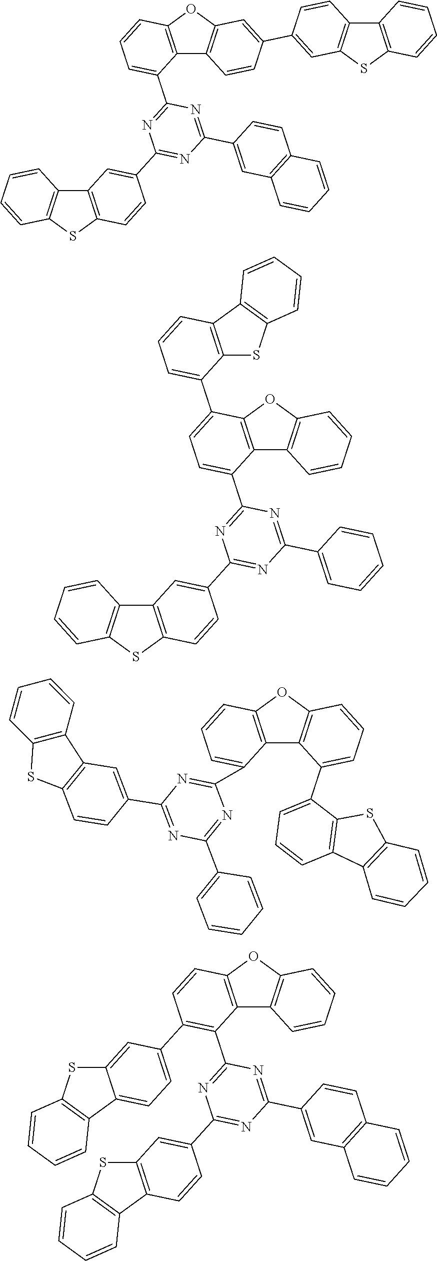

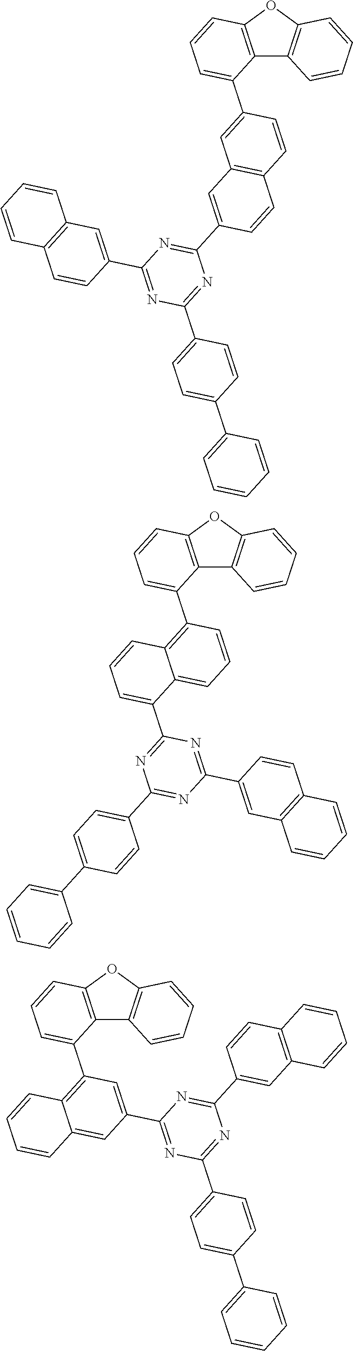

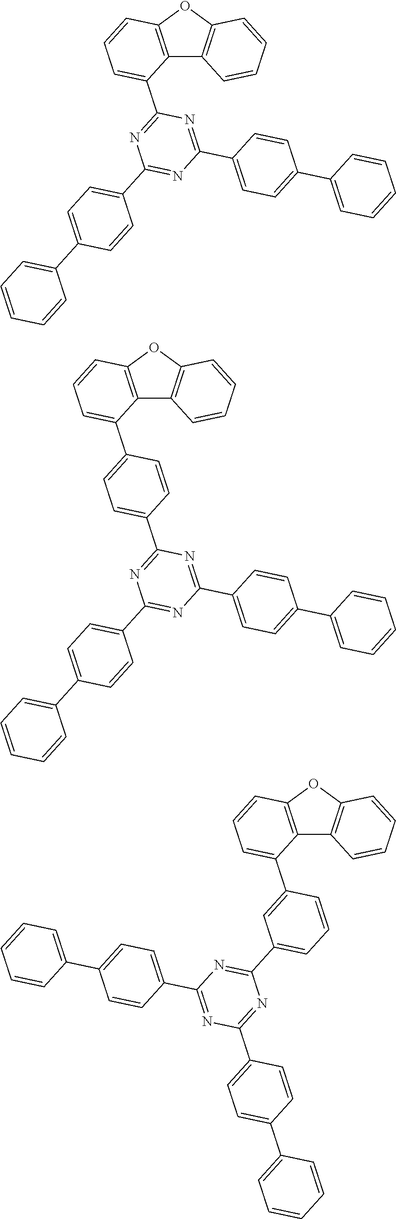 [SMILES: c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4ccc(-c5ccccc5)cc4)nc(-c4ccc(-c5cccc6oc7ccccc7c56)cc4)n3)cc2)cc1 ; c1ccc(-c2ccc(-c3nc(-c4ccc(-c5ccccc5)cc4)nc(-c4cccc(-c5cccc6oc7ccccc7c56)c4)n3)cc2)cc1]

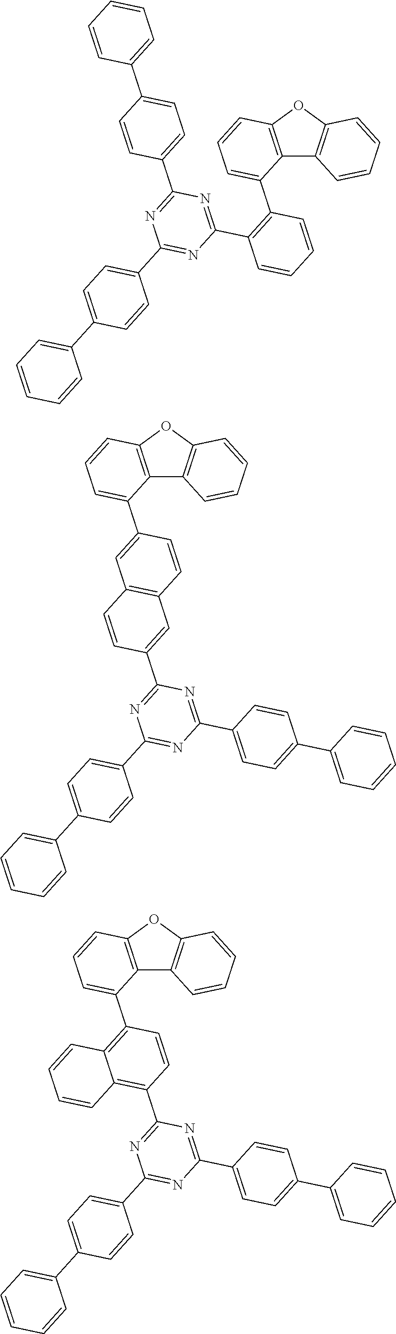 [SMILES: c1ccc(-c2ccc(-c3nc(-c4ccc(-c5ccccc5)cc4)nc(-c4ccccc4-c4cccc5oc6ccccc6c45)n3)cc2)cc1 ; c1ccc(-c2ccc(-c3nc(-c4ccc(-c5ccccc5)cc4)nc(-c4ccc5cc(-c6cccc7oc8ccccc8c67)ccc5c4)n3)cc2)cc1 ; c1ccc(-c2ccc(-c3nc(-c4ccc(-c5ccccc5)cc4)nc(-c4ccc(-c5cccc6oc7ccccc7c56)c5ccccc45)n3)cc2)cc1]

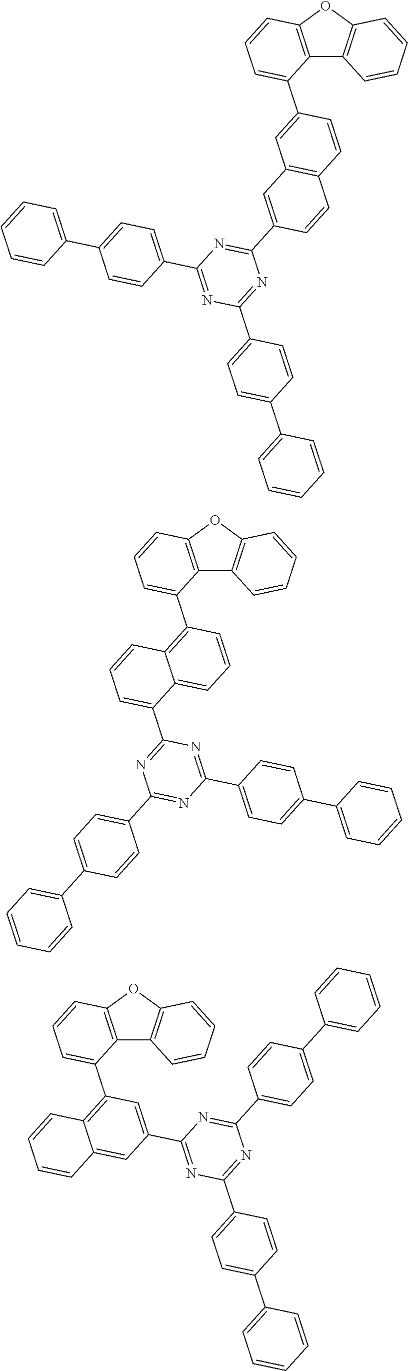

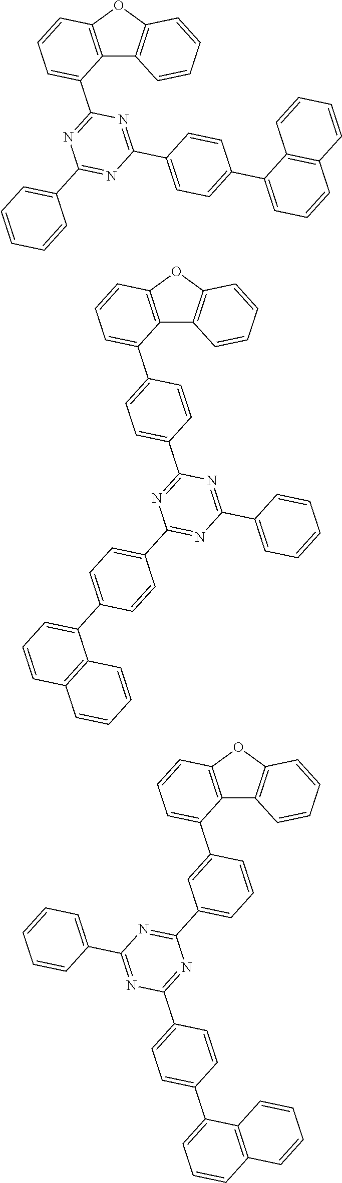 [SMILES: c1ccc(-c2nc(-c3ccc(-c4cccc5ccccc45)cc3)nc(-c3cccc4oc5ccccc5c34)n2)cc1 ; c1ccc(-c2nc(-c3ccc(-c4cccc5ccccc45)cc3)nc(-c3ccc(-c4cccc5oc6ccccc6c45)cc3)n2)cc1 ; c1ccc(-c2nc(-c3ccc(-c4cccc5ccccc45)cc3)nc(-c3cccc(-c4cccc5oc6ccccc6c45)c3)n2)cc1]

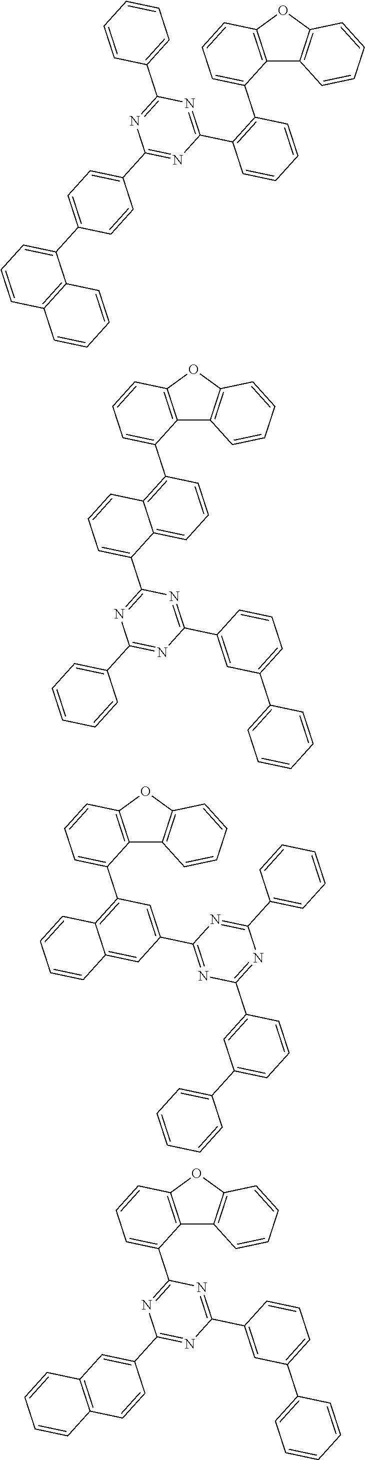

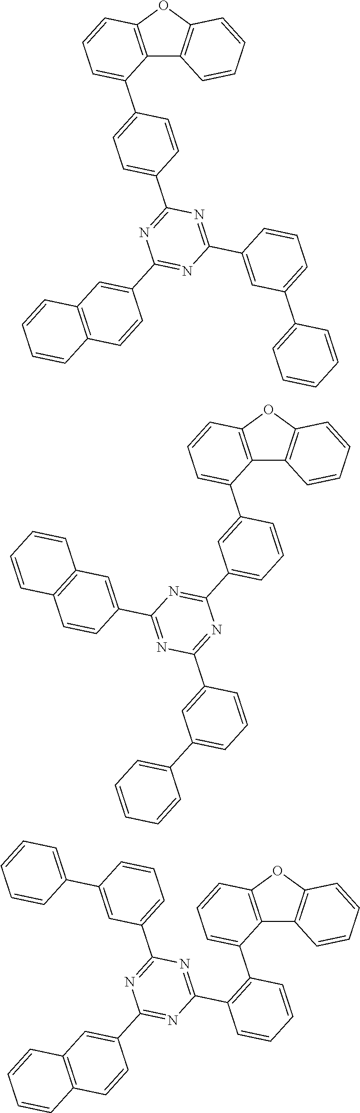

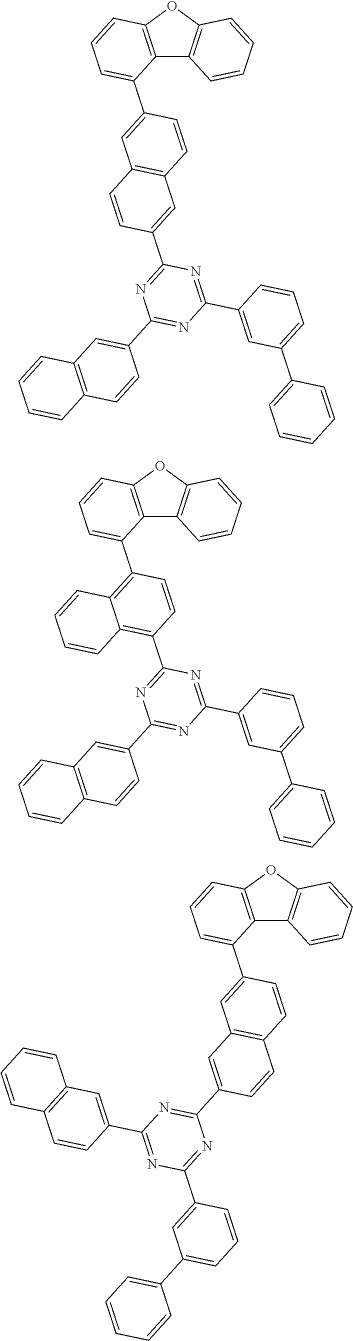

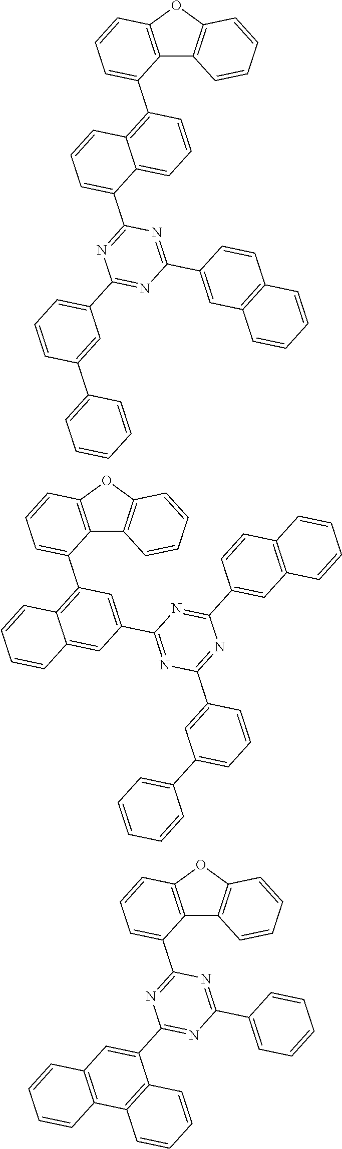

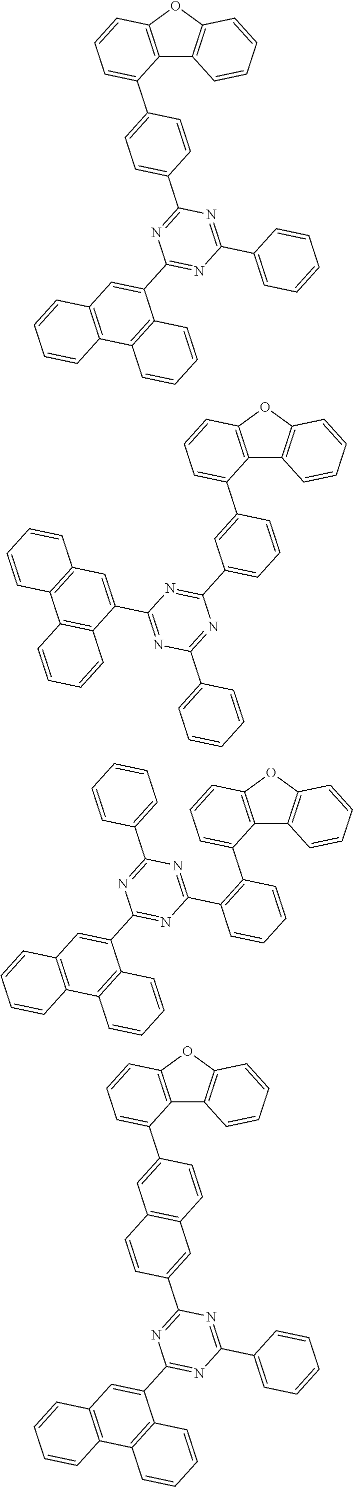

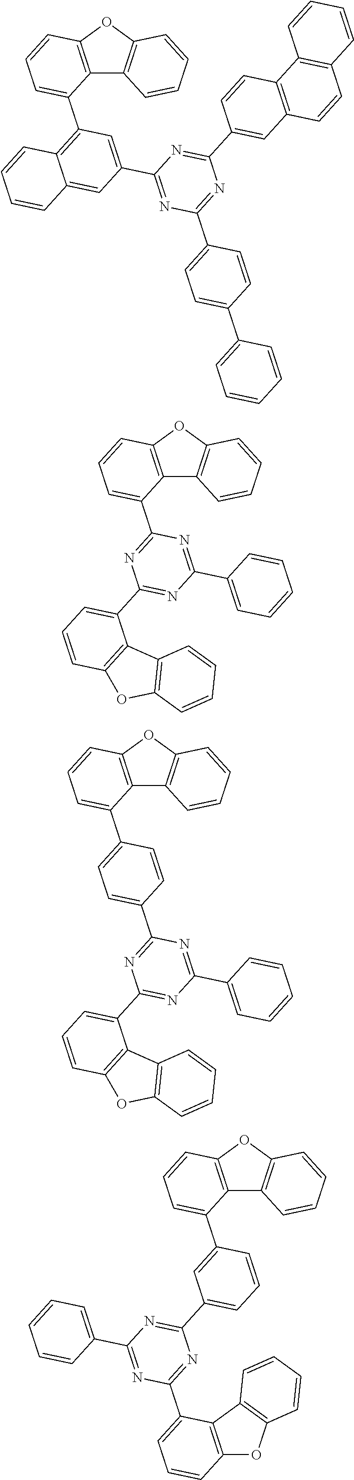

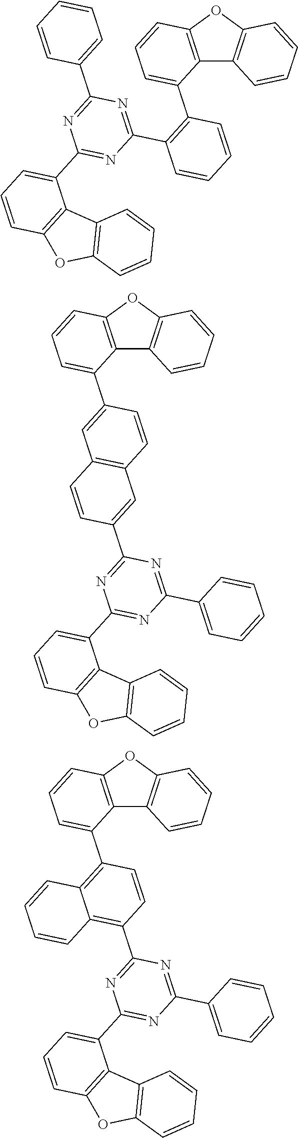

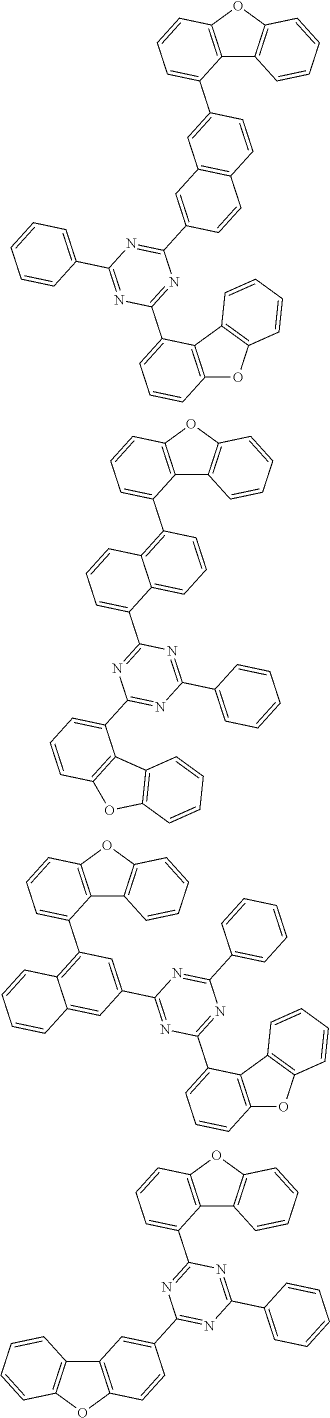

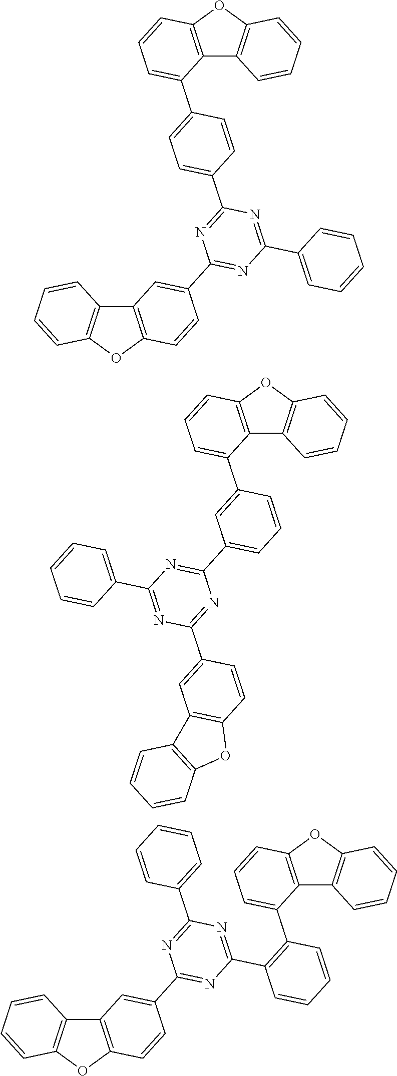

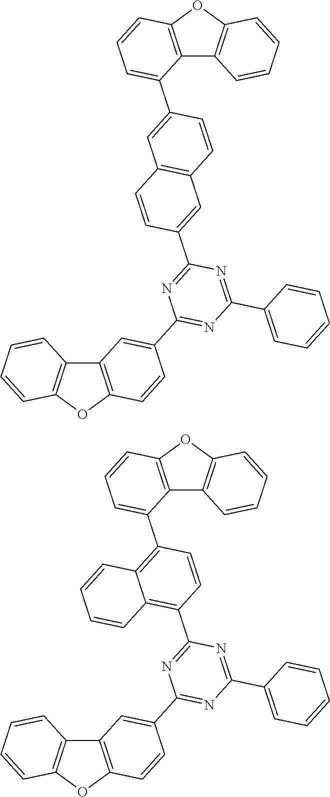 [SMILES: c1ccc(-c2nc(-c3ccc4cc(-c5cccc6oc7ccccc7c56)ccc4c3)nc(-c3ccc4oc5ccccc5c4c3)n2)cc1 ; c1ccc(-c2nc(-c3ccc4oc5ccccc5c4c3)nc(-c3ccc(-c4cccc5oc6ccccc6c45)c4ccccc34)n2)cc1]

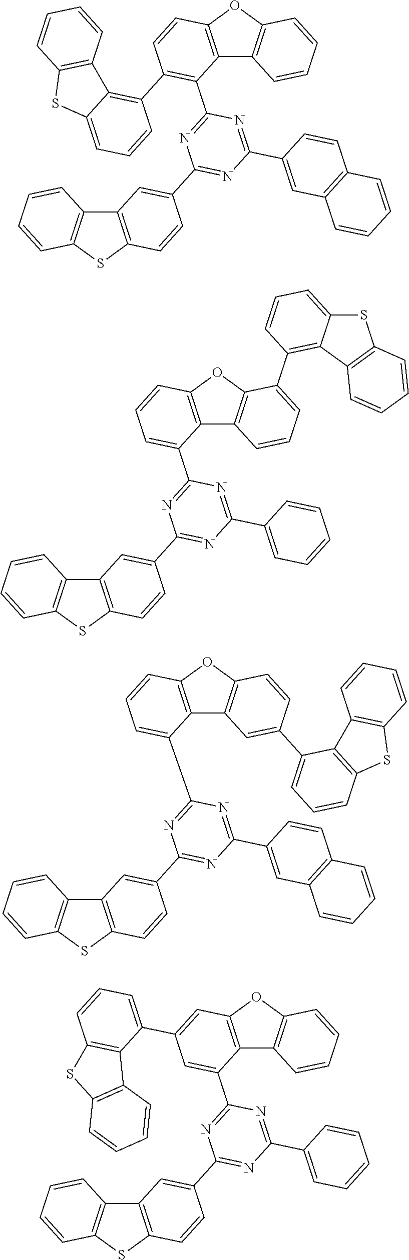

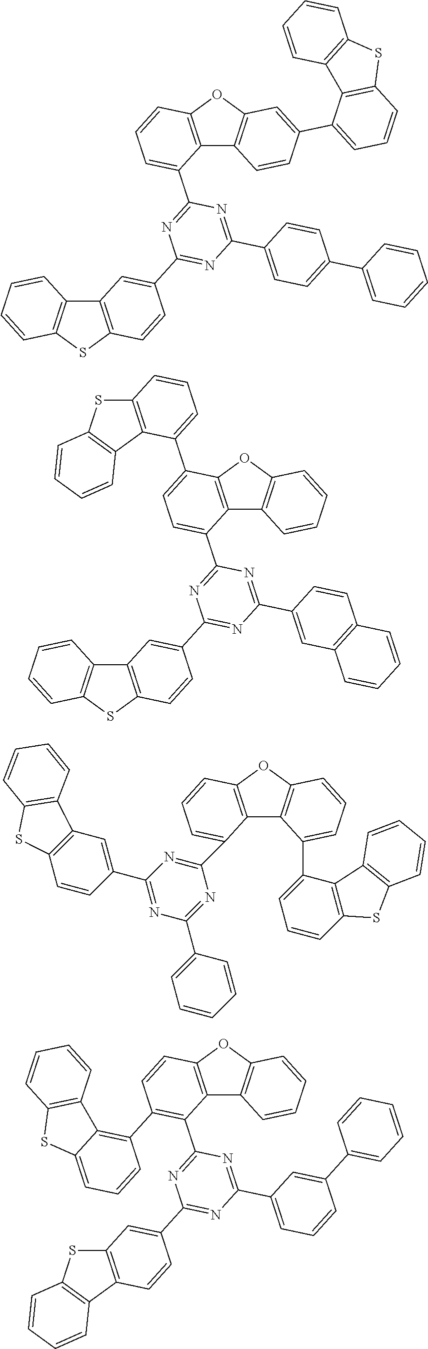

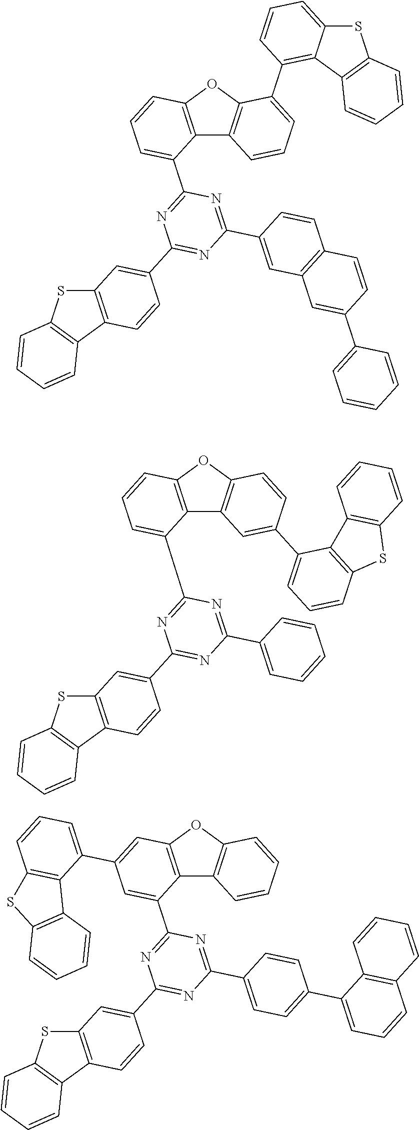

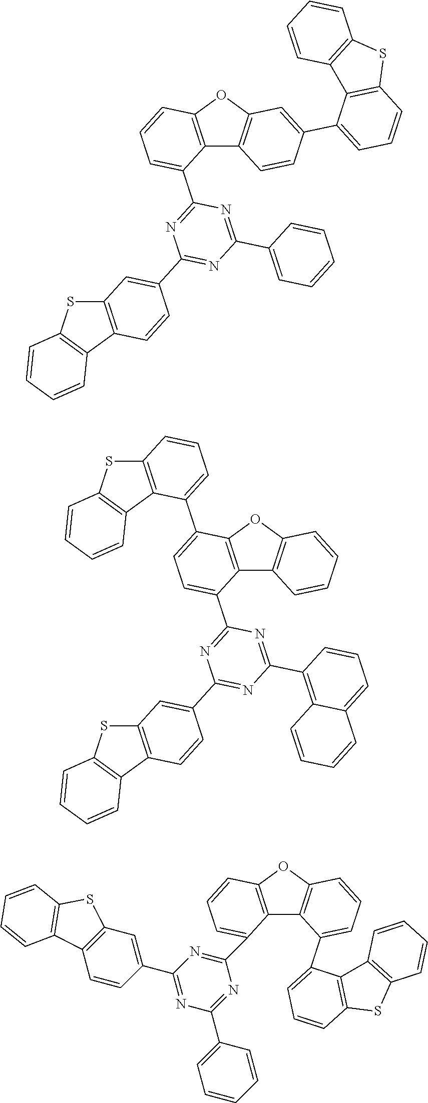 [SMILES: c1ccc(-c2nc(-c3ccc4c(c3)sc3ccccc34)nc(-c3cccc4oc5cc(-c6cccc7sc8ccccc8c67)ccc5c34)n2)cc1 ; c1ccc2c(-c3nc(-c4ccc5c(c4)sc4ccccc45)nc(-c4ccc(-c5cccc6sc7ccccc7c56)c5oc6ccccc6c45)n3)cccc2c1 ; c1ccc(-c2nc(-c3ccc4c(c3)sc3ccccc34)nc(-c3cccc4oc5cccc(-c6cccc7sc8ccccc8c67)c5c34)n2)cc1]

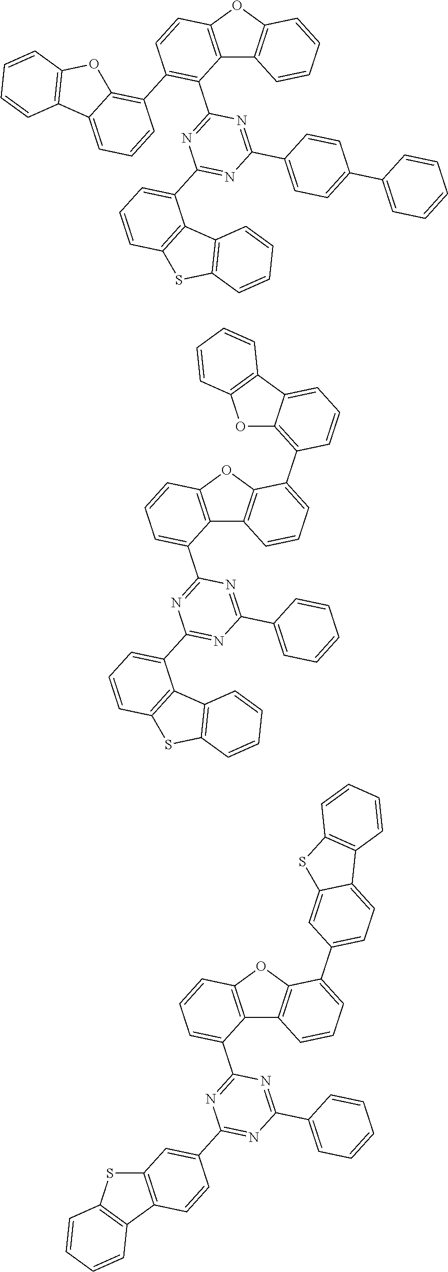

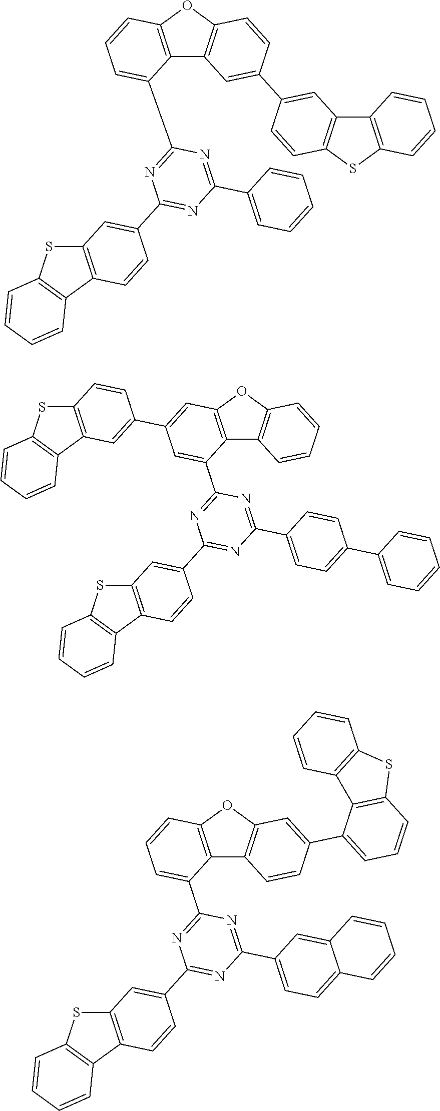 [SMILES: c1ccc(-c2nc(-c3ccc4c(c3)sc3ccccc34)nc(-c3cccc4oc5ccc(-c6ccc7sc8ccccc8c7c6)cc5c34)n2)cc1 ; c1ccc(-c2ccc(-c3nc(-c4ccc5c(c4)sc4ccccc45)nc(-c4cc(-c5ccc6sc7ccccc7c6c5)cc5oc6ccccc6c45)n3)cc2)cc1 ; c1ccc2cc(-c3nc(-c4ccc5c(c4)sc4ccccc45)nc(-c4cccc5oc6cc(-c7cccc8sc9ccccc9c78)ccc6c45)n3)ccc2c1]

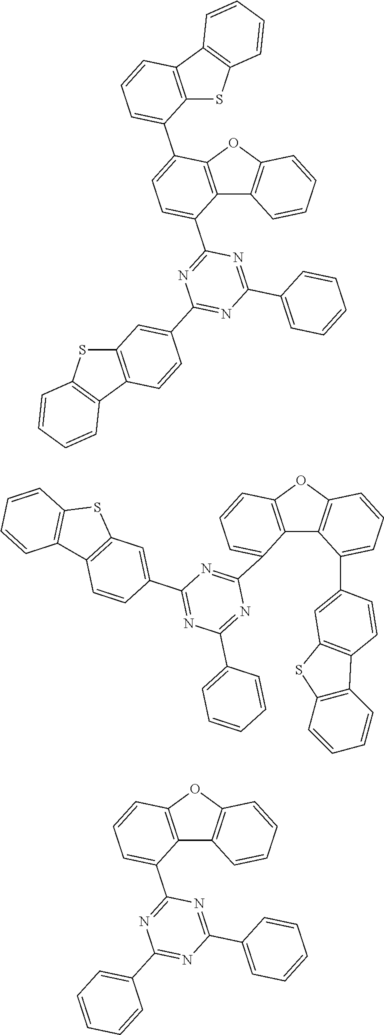 [SMILES: c1ccc(-c2nc(-c3ccc4c(c3)sc3ccccc34)nc(-c3ccc(-c4cccc5c4sc4ccccc45)c4oc5ccccc5c34)n2)cc1 ; c1ccc(-c2nc(-c3ccc4c(c3)sc3ccccc34)nc(-c3cccc4oc5cccc(-c6ccc7c(c6)sc6ccccc67)c5c34)n2)cc1 ; c1ccc(-c2nc(-c3ccccc3)nc(-c3cccc4oc5ccccc5c34)n2)cc1]

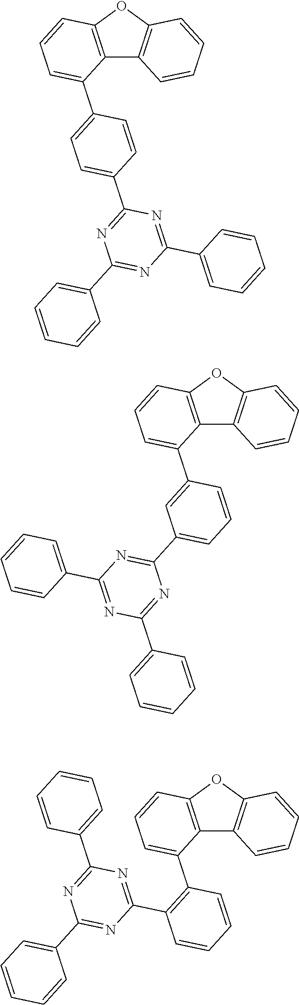 [SMILES: c1ccc(-c2nc(-c3ccccc3)nc(-c3ccc(-c4cccc5oc6ccccc6c45)cc3)n2)cc1 ; c1ccc(-c2nc(-c3ccccc3)nc(-c3cccc(-c4cccc5oc6ccccc6c45)c3)n2)cc1 ; c1ccc(-c2nc(-c3ccccc3)nc(-c3ccccc3-c3cccc4oc5ccccc5c34)n2)cc1]

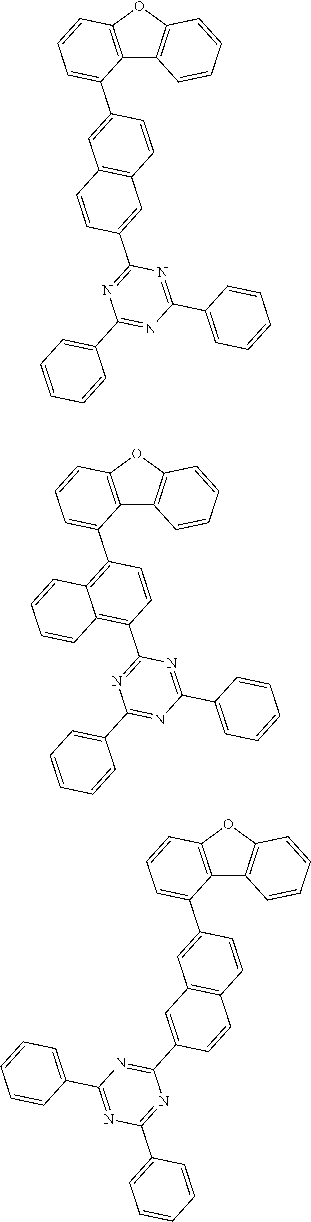 [SMILES: c1ccc(-c2nc(-c3ccccc3)nc(-c3ccc4cc(-c5cccc6oc7ccccc7c56)ccc4c3)n2)cc1 ; c1ccc(-c2nc(-c3ccccc3)nc(-c3ccc(-c4cccc5oc6ccccc6c45)c4ccccc34)n2)cc1 ; c1ccc(-c2nc(-c3ccccc3)nc(-c3ccc4ccc(-c5cccc6oc7ccccc7c56)cc4c3)n2)cc1]

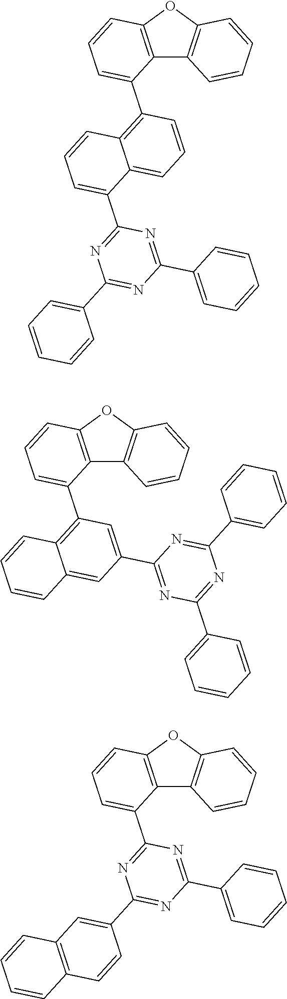 [SMILES: c1ccc(-c2nc(-c3ccccc3)nc(-c3cccc4c(-c5cccc6oc7ccccc7c56)cccc34)n2)cc1 ; c1ccc(-c2nc(-c3ccccc3)nc(-c3cc(-c4cccc5oc6ccccc6c45)c4ccccc4c3)n2)cc1 ; c1ccc(-c2nc(-c3ccc4ccccc4c3)nc(-c3cccc4oc5ccccc5c34)n2)cc1]

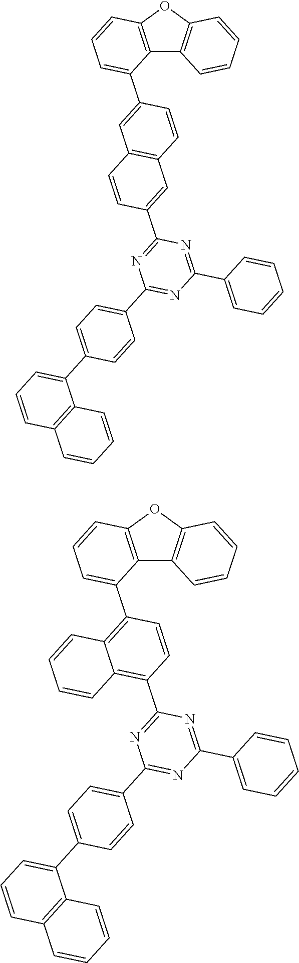 [SMILES: c1ccc(-c2nc(-c3ccc(-c4cccc5ccccc45)cc3)nc(-c3ccc4cc(-c5cccc6oc7ccccc7c56)ccc4c3)n2)cc1 ; c1ccc(-c2nc(-c3ccc(-c4cccc5ccccc45)cc3)nc(-c3ccc(-c4cccc5oc6ccccc6c45)c4ccccc34)n2)cc1]

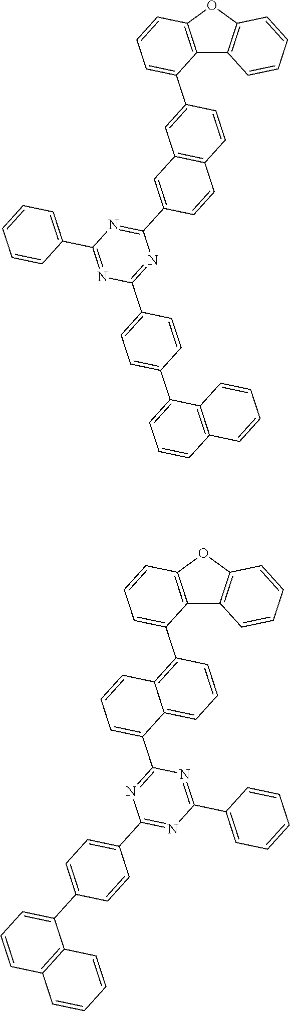 [SMILES: c1ccc(-c2nc(-c3ccc(-c4cccc5ccccc45)cc3)nc(-c3ccc4ccc(-c5cccc6oc7ccccc7c56)cc4c3)n2)cc1 ; c1ccc(-c2nc(-c3ccc(-c4cccc5ccccc45)cc3)nc(-c3cccc4c(-c5cccc6oc7ccccc7c56)cccc34)n2)cc1]

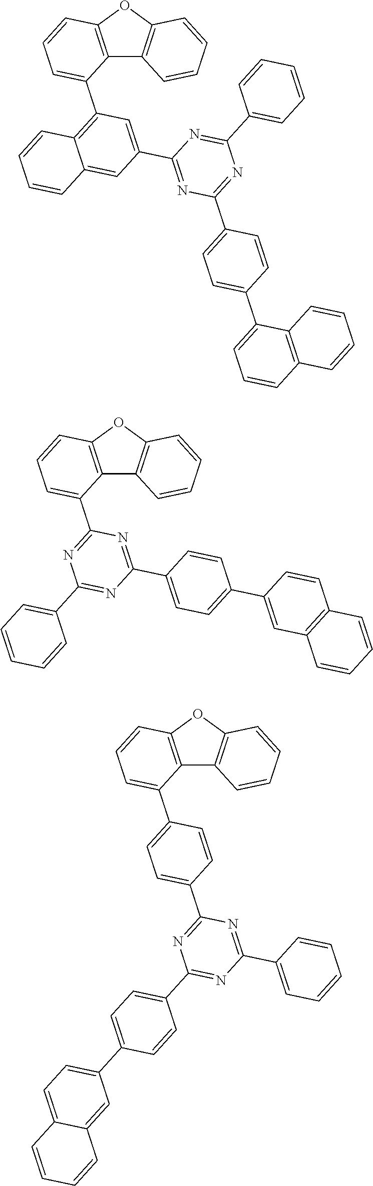 [SMILES: c1ccc(-c2nc(-c3ccc(-c4cccc5ccccc45)cc3)nc(-c3cc(-c4cccc5oc6ccccc6c45)c4ccccc4c3)n2)cc1 ; c1ccc(-c2nc(-c3ccc(-c4ccc5ccccc5c4)cc3)nc(-c3cccc4oc5ccccc5c34)n2)cc1 ; c1ccc(-c2nc(-c3ccc(-c4ccc5ccccc5c4)cc3)nc(-c3ccc(-c4cccc5oc6ccccc6c45)cc3)n2)cc1]

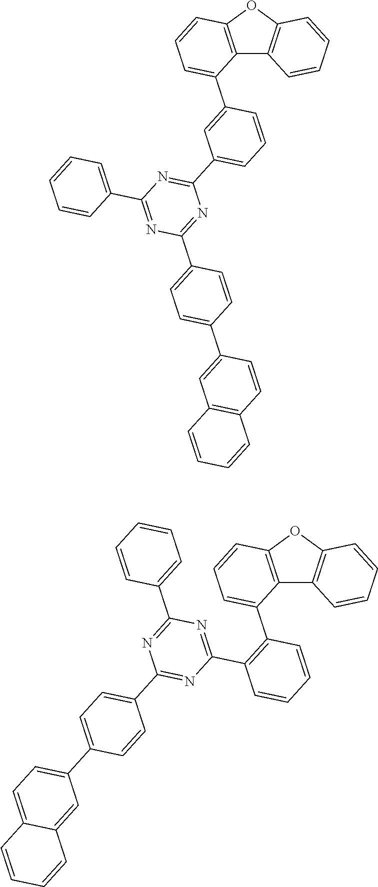 [SMILES: c1ccc(-c2nc(-c3ccc(-c4ccc5ccccc5c4)cc3)nc(-c3cccc(-c4cccc5oc6ccccc6c45)c3)n2)cc1 ; c1ccc(-c2nc(-c3ccc(-c4ccc5ccccc5c4)cc3)nc(-c3ccccc3-c3cccc4oc5ccccc5c34)n2)cc1]

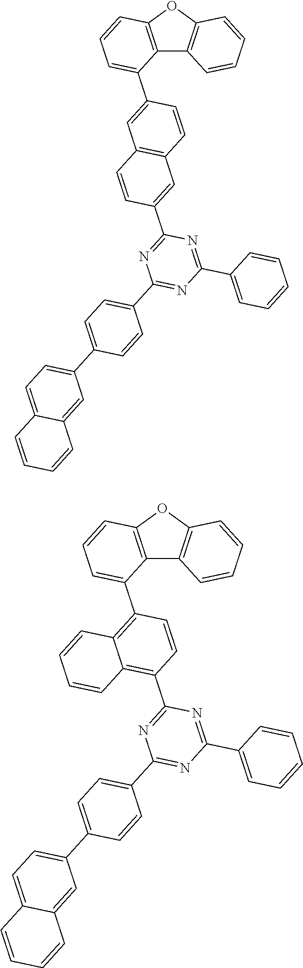 [SMILES: c1ccc(-c2nc(-c3ccc(-c4ccc5ccccc5c4)cc3)nc(-c3ccc4cc(-c5cccc6oc7ccccc7c56)ccc4c3)n2)cc1 ; c1ccc(-c2nc(-c3ccc(-c4ccc5ccccc5c4)cc3)nc(-c3ccc(-c4cccc5oc6ccccc6c45)c4ccccc34)n2)cc1]

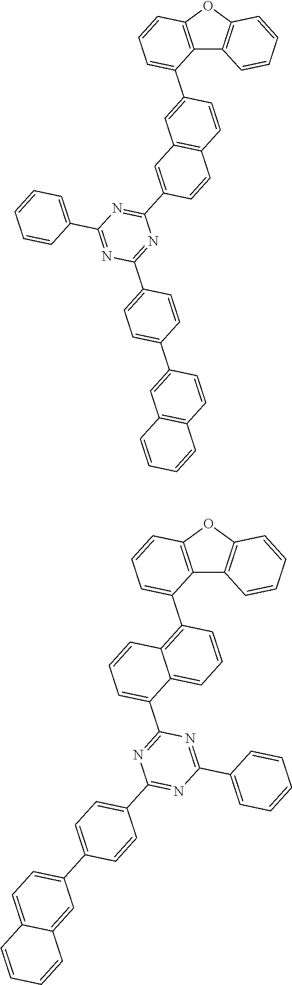 [SMILES: c1ccc(-c2nc(-c3ccc(-c4ccc5ccccc5c4)cc3)nc(-c3ccc4ccc(-c5cccc6oc7ccccc7c56)cc4c3)n2)cc1 ; c1ccc(-c2nc(-c3ccc(-c4ccc5ccccc5c4)cc3)nc(-c3cccc4c(-c5cccc6oc7ccccc7c56)cccc34)n2)cc1]

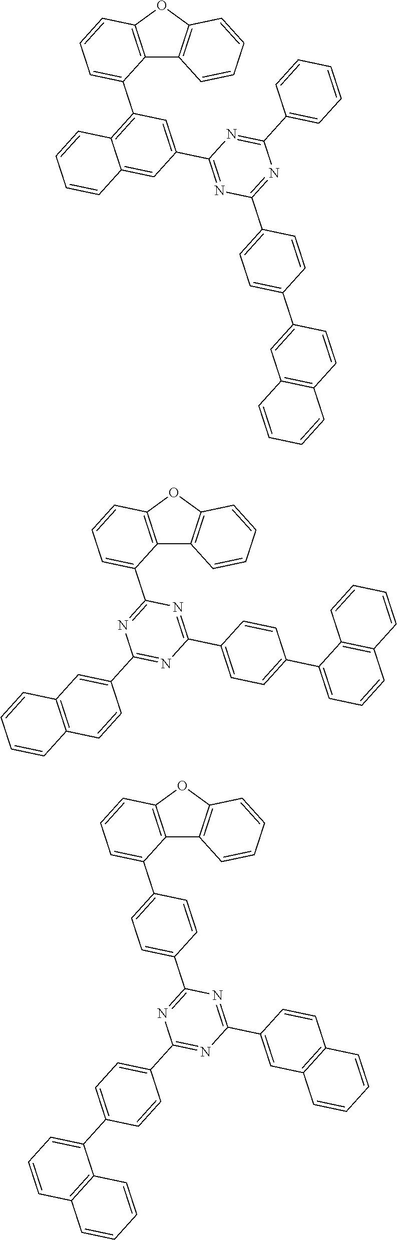 [SMILES: c1ccc(-c2nc(-c3ccc(-c4ccc5ccccc5c4)cc3)nc(-c3cc(-c4cccc5oc6ccccc6c45)c4ccccc4c3)n2)cc1 ; c1ccc2cc(-c3nc(-c4ccc(-c5cccc6ccccc56)cc4)nc(-c4cccc5oc6ccccc6c45)n3)ccc2c1 ; c1ccc2cc(-c3nc(-c4ccc(-c5cccc6ccccc56)cc4)nc(-c4ccc(-c5cccc6oc7ccccc7c56)cc4)n3)ccc2c1]

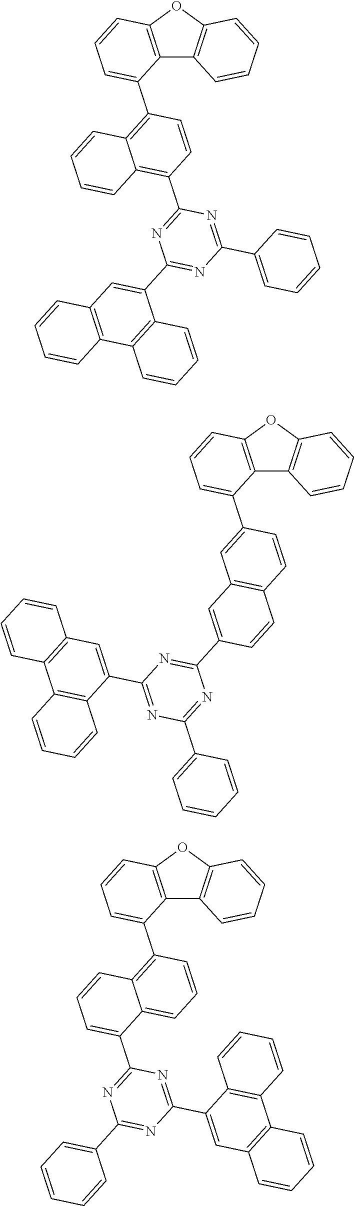

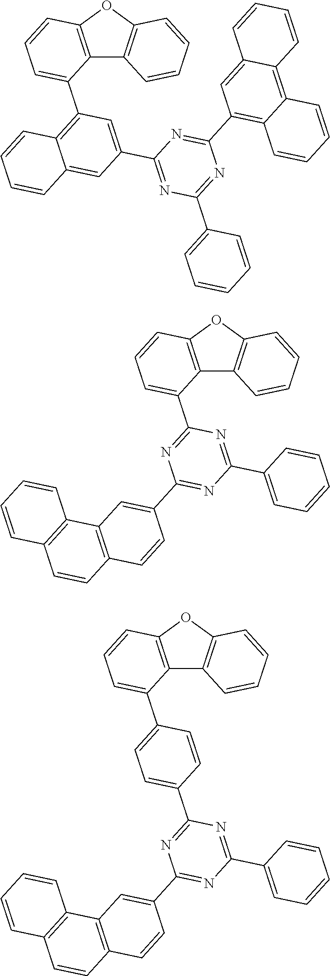

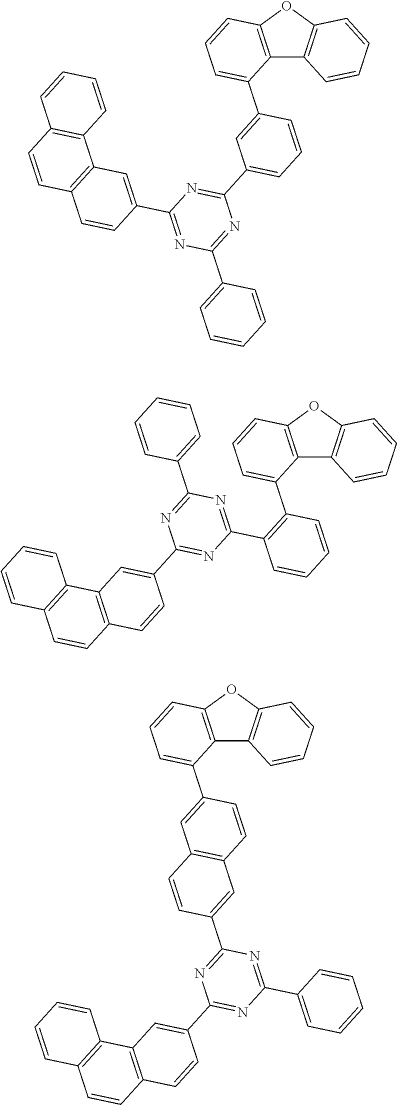 [SMILES: c1ccc(-c2nc(-c3cccc(-c4cccc5oc6ccccc6c45)c3)nc(-c3ccc4ccc5ccccc5c4c3)n2)cc1 ; c1ccc(-c2nc(-c3ccc4ccc5ccccc5c4c3)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4ccc5ccccc5c4c3)n2)cc1]

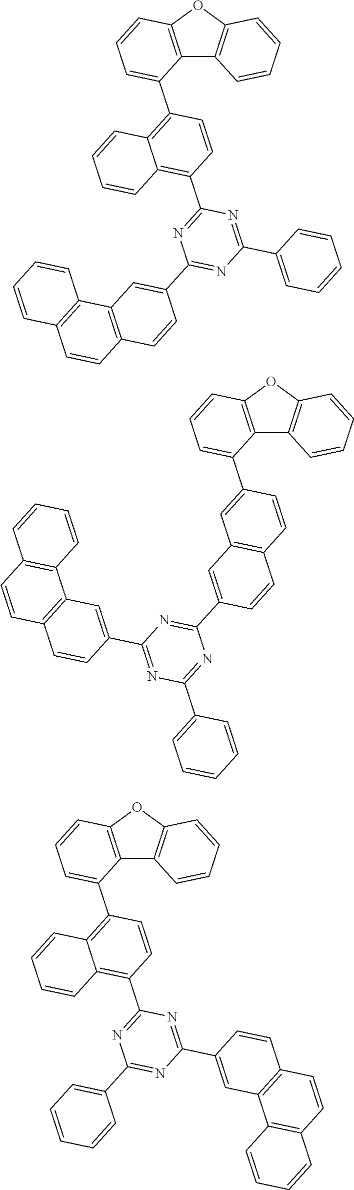

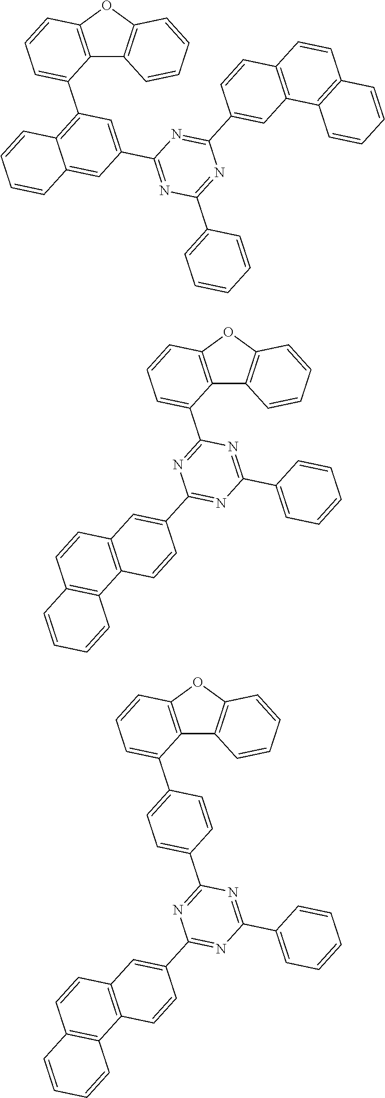

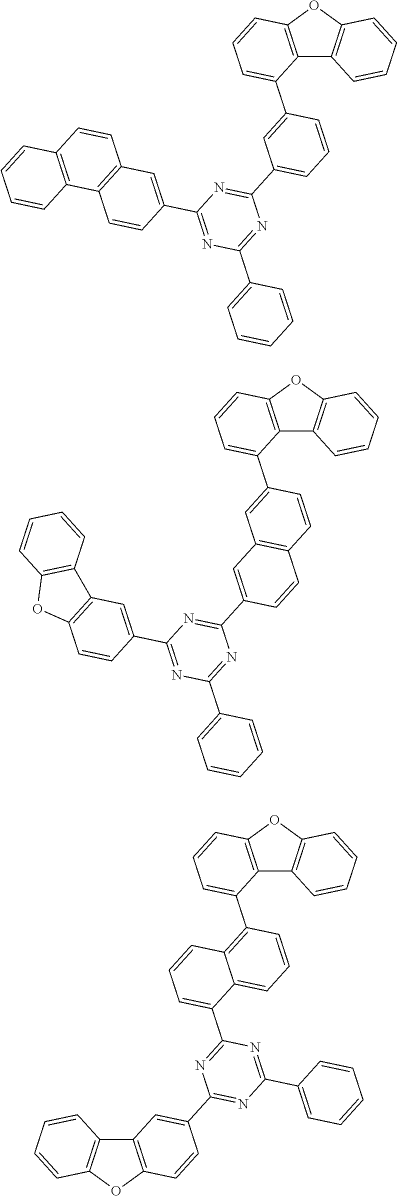

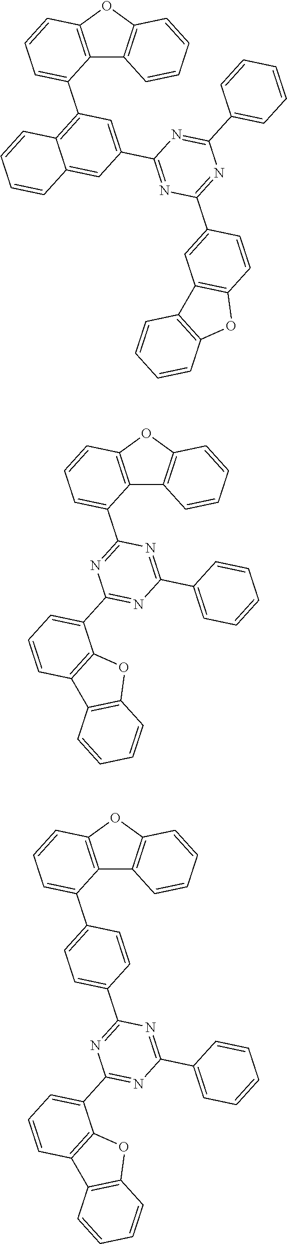 [SMILES: c1ccc(-c2nc(-c3cc(-c4cccc5oc6ccccc6c45)c4ccccc4c3)nc(-c3ccc4oc5ccccc5c4c3)n2)cc1 ; c1ccc(-c2nc(-c3cccc4c3oc3ccccc34)nc(-c3cccc4oc5ccccc5c34)n2)cc1 ; c1ccc(-c2nc(-c3ccc(-c4cccc5oc6ccccc6c45)cc3)nc(-c3cccc4c3oc3ccccc34)n2)cc1]

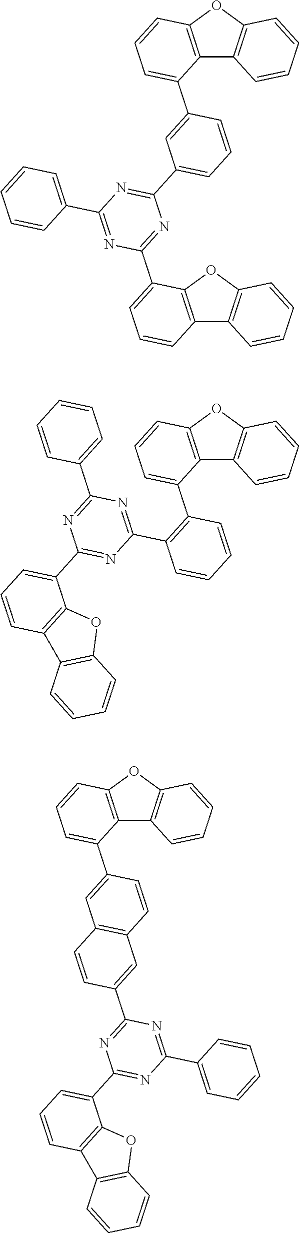 [SMILES: c1ccc(-c2nc(-c3cccc(-c4cccc5oc6ccccc6c45)c3)nc(-c3cccc4c3oc3ccccc34)n2)cc1 ; c1ccc(-c2nc(-c3ccccc3-c3cccc4oc5ccccc5c34)nc(-c3cccc4c3oc3ccccc34)n2)cc1 ; c1ccc(-c2nc(-c3ccc4cc(-c5cccc6oc7ccccc7c56)ccc4c3)nc(-c3cccc4c3oc3ccccc34)n2)cc1]

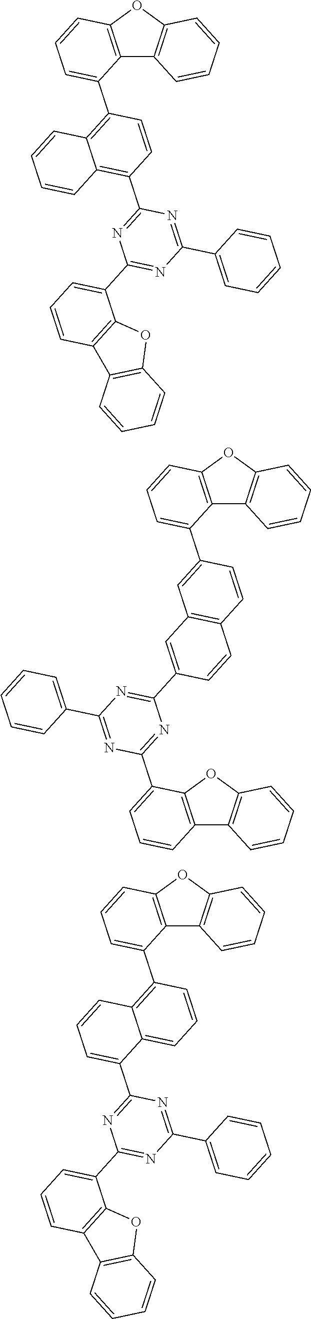

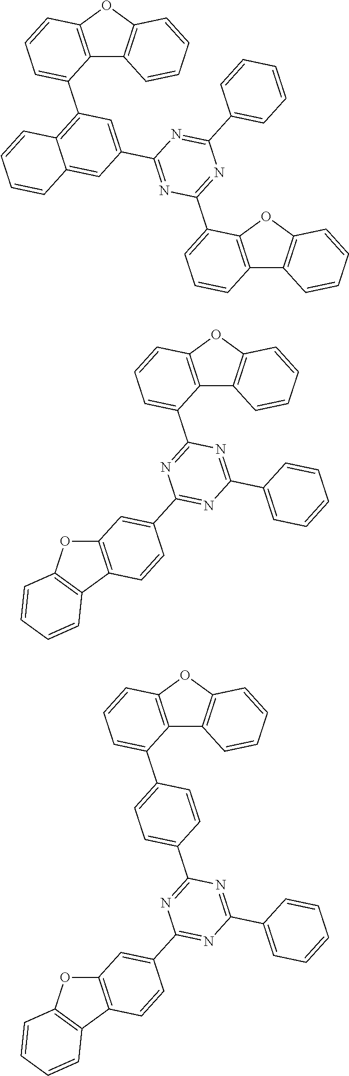

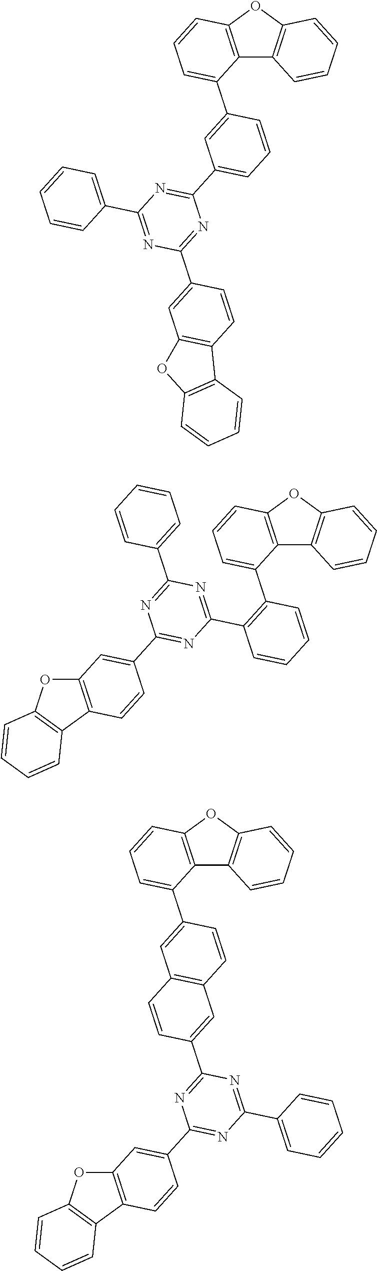 [SMILES: c1ccc(-c2nc(-c3cccc(-c4cccc5oc6ccccc6c45)c3)nc(-c3ccc4c(c3)oc3ccccc34)n2)cc1 ; c1ccc(-c2nc(-c3ccc4c(c3)oc3ccccc34)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4c(c3)oc3ccccc34)n2)cc1]

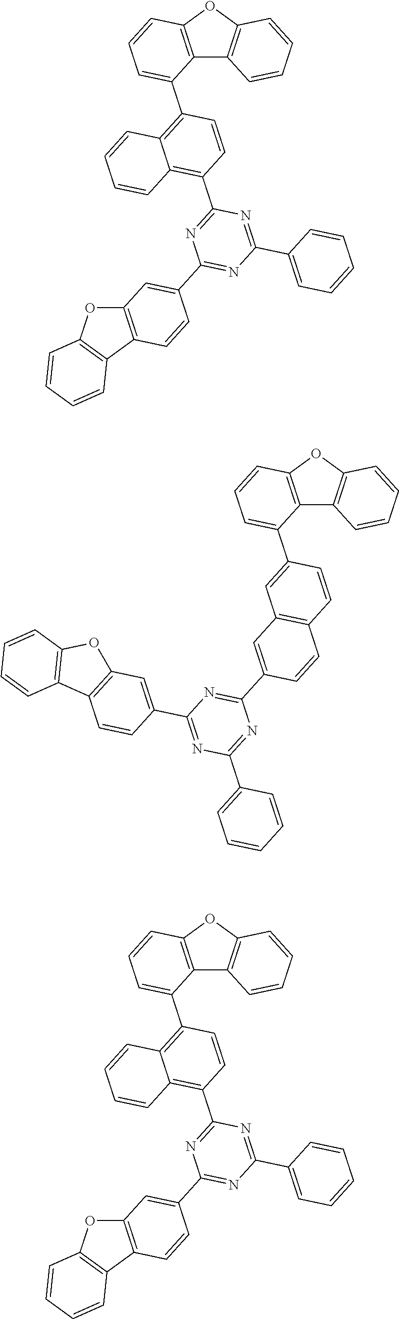 [SMILES: c1ccc(-c2nc(-c3ccc4c(c3)oc3ccccc34)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4c(c3)oc3ccccc34)n2)cc1 ; c1ccc(-c2nc(-c3ccc4c(c3)oc3ccccc34)nc(-c3ccc(-c4cccc5oc6ccccc6c45)c4ccccc34)n2)cc1]

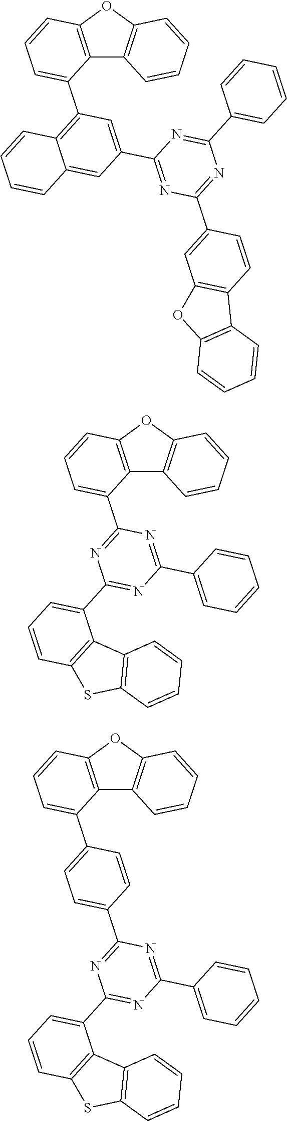

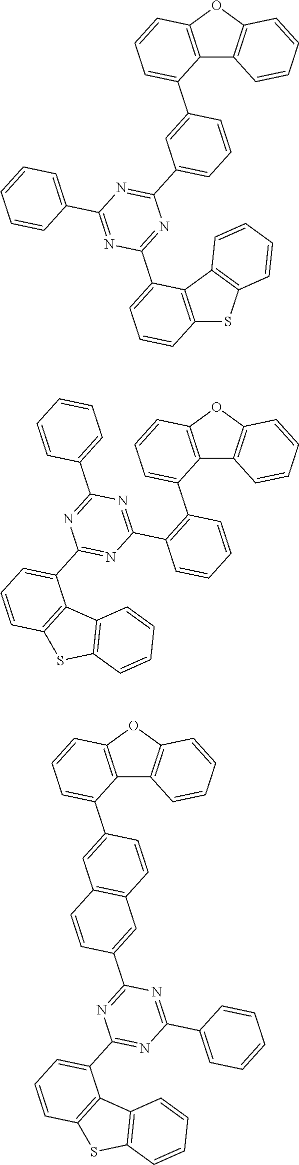 [SMILES: c1ccc(-c2nc(-c3cccc(-c4cccc5oc6ccccc6c45)c3)nc(-c3cccc4sc5ccccc5c34)n2)cc1 ; c1ccc(-c2nc(-c3ccccc3-c3cccc4oc5ccccc5c34)nc(-c3cccc4sc5ccccc5c34)n2)cc1 ; c1ccc(-c2nc(-c3ccc4cc(-c5cccc6oc7ccccc7c56)ccc4c3)nc(-c3cccc4sc5ccccc5c34)n2)cc1]

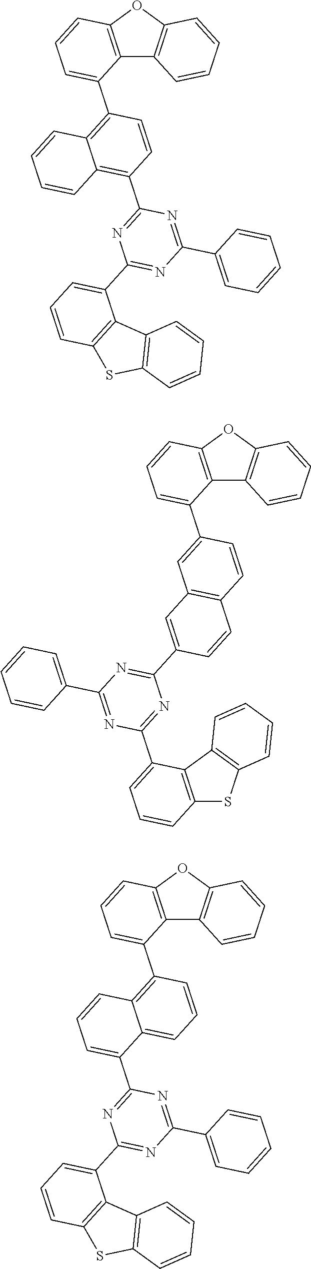

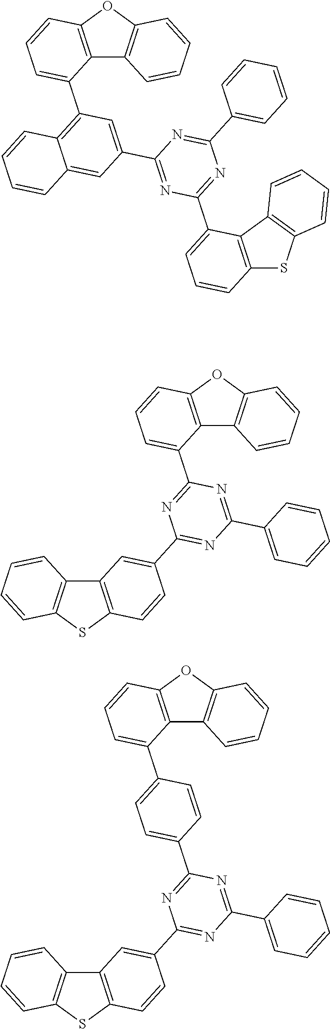 [SMILES: c1ccc(-c2nc(-c3cc(-c4cccc5oc6ccccc6c45)c4ccccc4c3)nc(-c3cccc4sc5ccccc5c34)n2)cc1 ; c1ccc(-c2nc(-c3ccc4sc5ccccc5c4c3)nc(-c3cccc4oc5ccccc5c34)n2)cc1 ; c1ccc(-c2nc(-c3ccc(-c4cccc5oc6ccccc6c45)cc3)nc(-c3ccc4sc5ccccc5c4c3)n2)cc1]

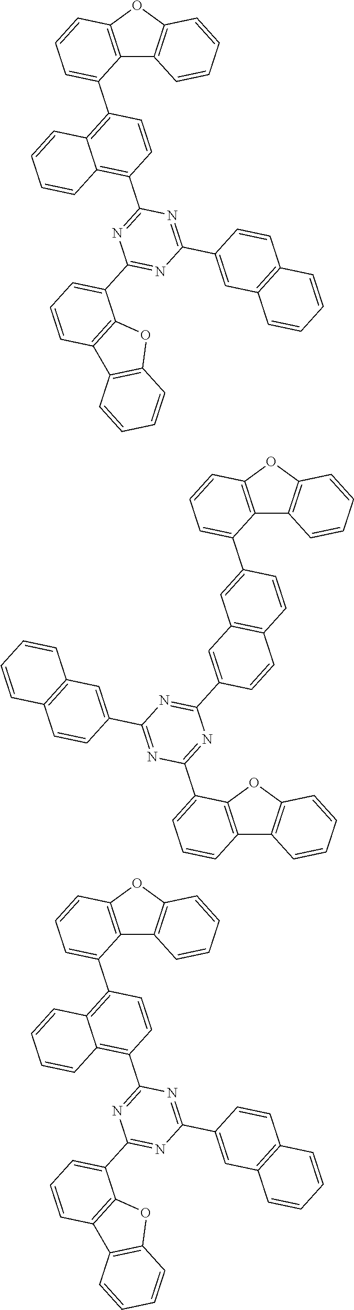

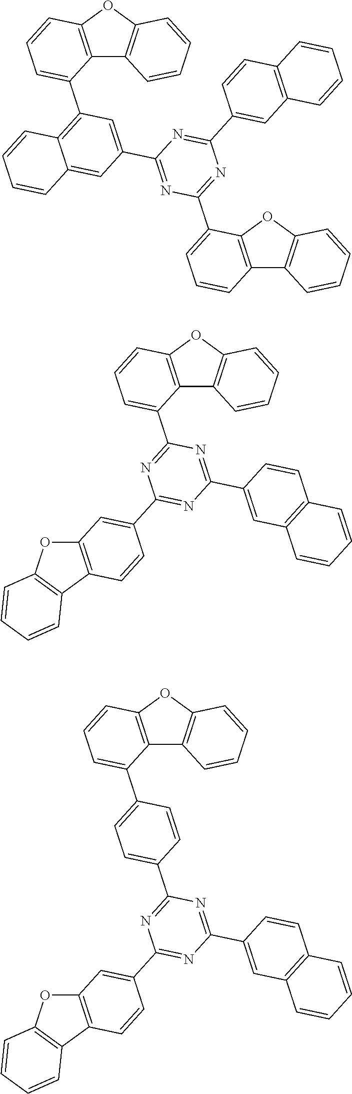 [SMILES: c1ccc2cc(-c3nc(-c4cc(-c5cccc6oc7ccccc7c56)c5ccccc5c4)nc(-c4cccc5c4oc4ccccc45)n3)ccc2c1 ; c1ccc2cc(-c3nc(-c4ccc5c(c4)oc4ccccc45)nc(-c4cccc5oc6ccccc6c45)n3)ccc2c1 ; c1ccc2cc(-c3nc(-c4ccc(-c5cccc6oc7ccccc7c56)cc4)nc(-c4ccc5c(c4)oc4ccccc45)n3)ccc2c1]

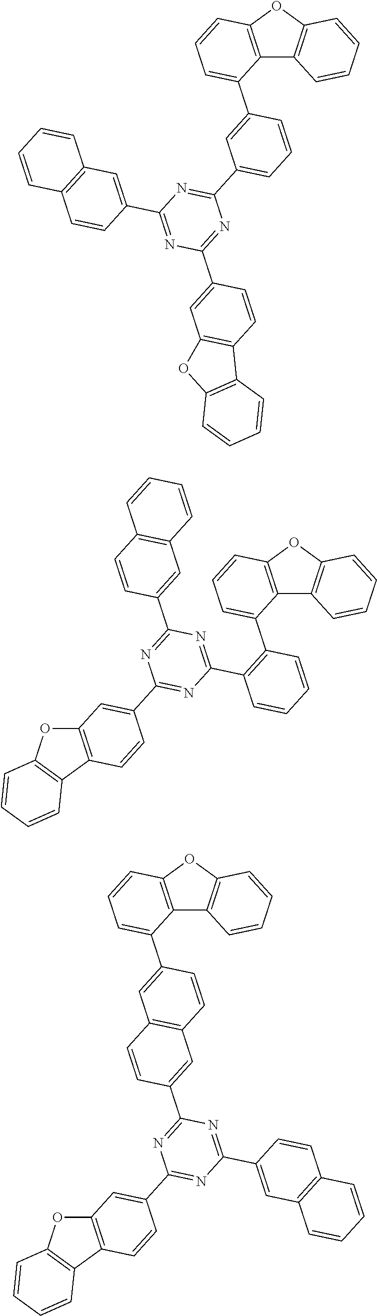 [SMILES: c1cc(-c2nc(-c3ccc4ccccc4c3)nc(-c3ccc4c(c3)oc3ccccc34)n2)cc(-c2cccc3oc4ccccc4c23)c1 ; c1ccc(-c2cccc3oc4ccccc4c23)c(-c2nc(-c3ccc4ccccc4c3)nc(-c3ccc4c(c3)oc3ccccc34)n2)c1 ; c1ccc2cc(-c3nc(-c4ccc5cc(-c6cccc7oc8ccccc8c67)ccc5c4)nc(-c4ccc5c(c4)oc4ccccc45)n3)ccc2c1]

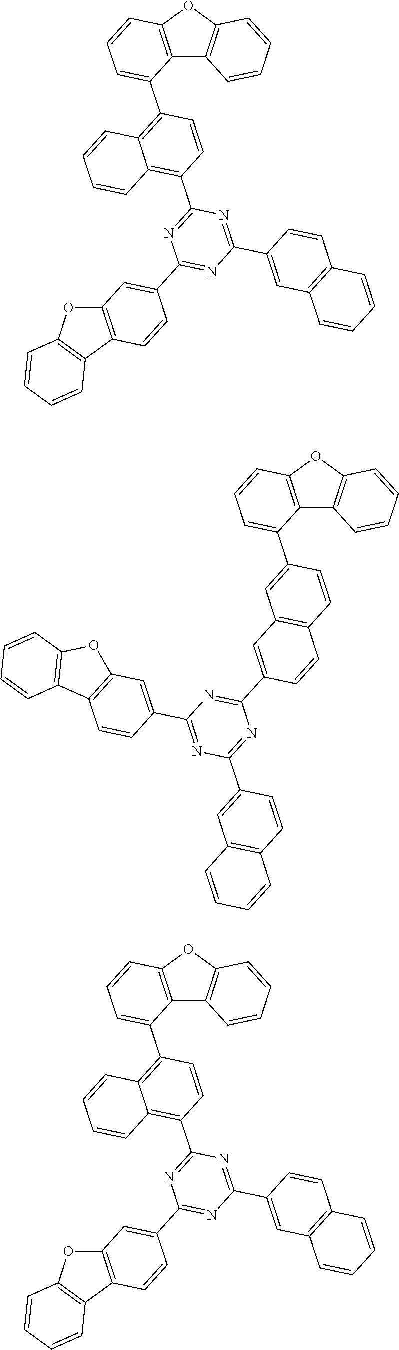 [SMILES: c1ccc2cc(-c3nc(-c4ccc5c(c4)oc4ccccc45)nc(-c4ccc(-c5cccc6oc7ccccc7c56)c5ccccc45)n3)ccc2c1 ; c1ccc2cc(-c3nc(-c4ccc5ccc(-c6cccc7oc8ccccc8c67)cc5c4)nc(-c4ccc5c(c4)oc4ccccc45)n3)ccc2c1 ; c1ccc2cc(-c3nc(-c4ccc5c(c4)oc4ccccc45)nc(-c4ccc(-c5cccc6oc7ccccc7c56)c5ccccc45)n3)ccc2c1]

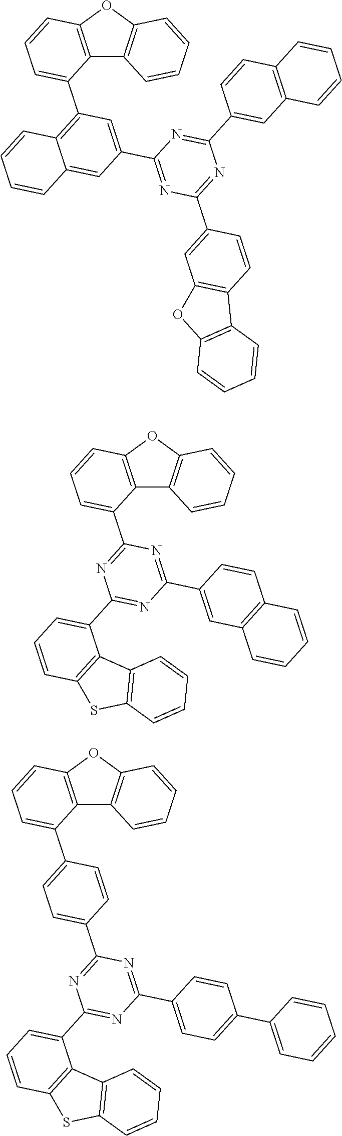

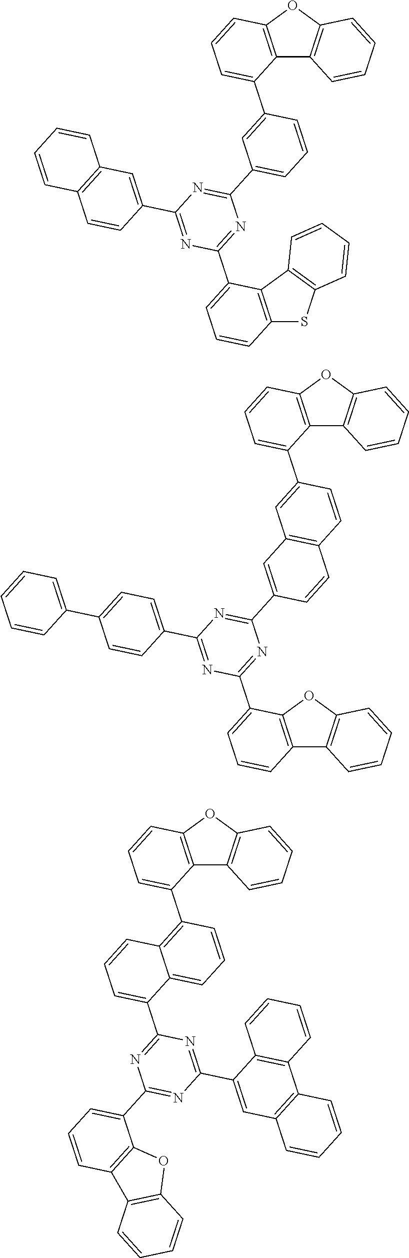

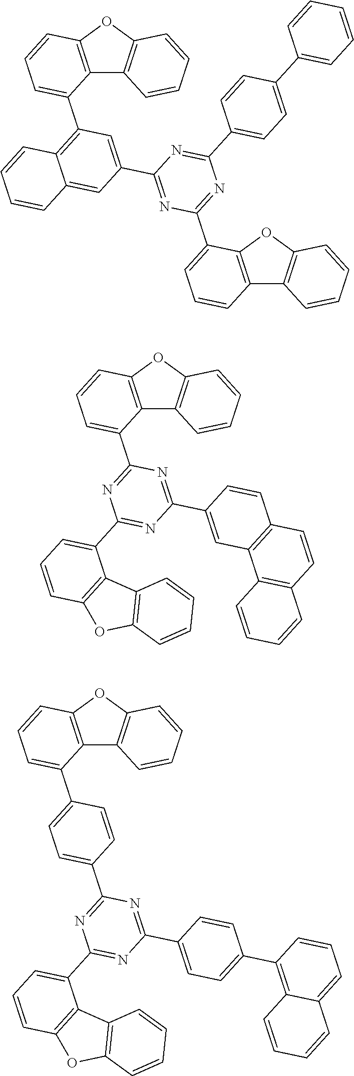 [SMILES: c1ccc(-c2ccc(-c3nc(-c4cc(-c5cccc6oc7ccccc7c56)c5ccccc5c4)nc(-c4cccc5c4oc4ccccc45)n3)cc2)cc1 ; c1ccc2c(c1)ccc1ccc(-c3nc(-c4cccc5oc6ccccc6c45)nc(-c4cccc5oc6ccccc6c45)n3)cc12 ; c1ccc2c(-c3ccc(-c4nc(-c5ccc(-c6cccc7oc8ccccc8c67)cc5)nc(-c5cccc6oc7ccccc7c56)n4)cc3)cccc2c1]

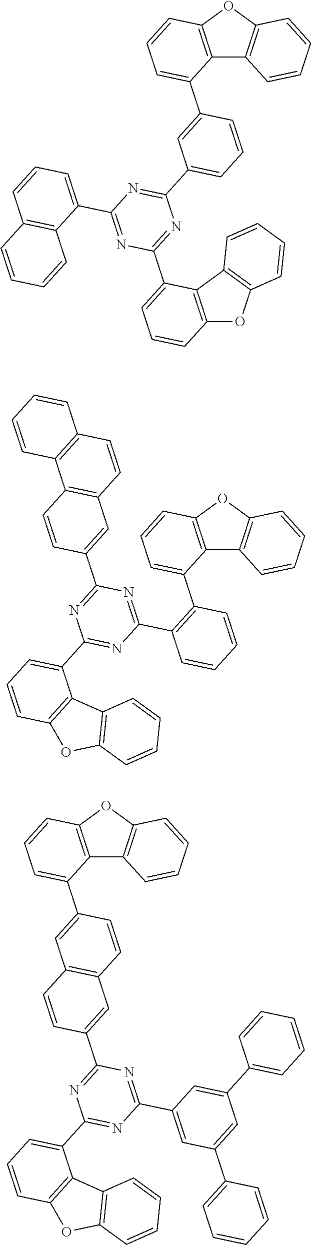

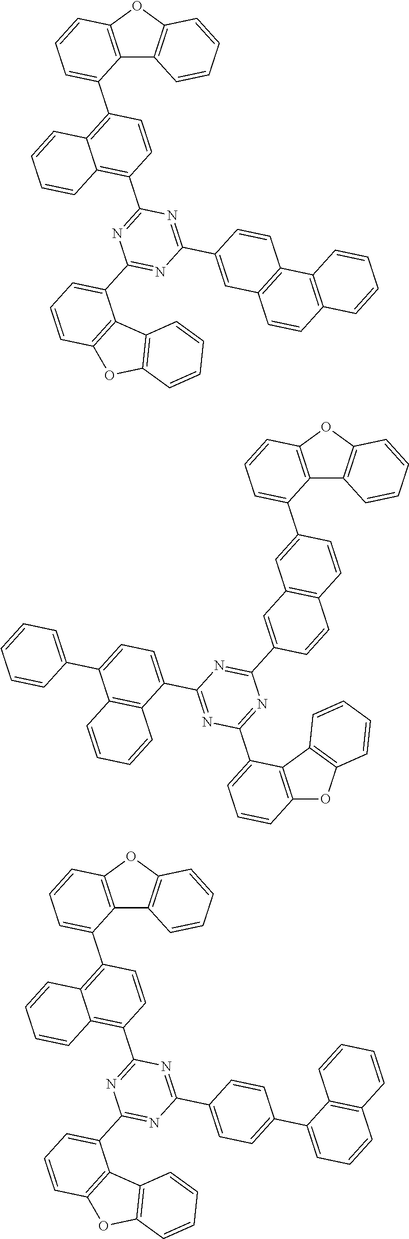 [SMILES: c1ccc2c(c1)ccc1cc(-c3nc(-c4ccc(-c5cccc6oc7ccccc7c56)c5ccccc45)nc(-c4cccc5oc6ccccc6c45)n3)ccc12 ; c1ccc(-c2ccc(-c3nc(-c4ccc5ccc(-c6cccc7oc8ccccc8c67)cc5c4)nc(-c4cccc5oc6ccccc6c45)n3)c3ccccc23)cc1 ; c1ccc2c(-c3ccc(-c4nc(-c5ccc(-c6cccc7oc8ccccc8c67)c6ccccc56)nc(-c5cccc6oc7ccccc7c56)n4)cc3)cccc2c1]

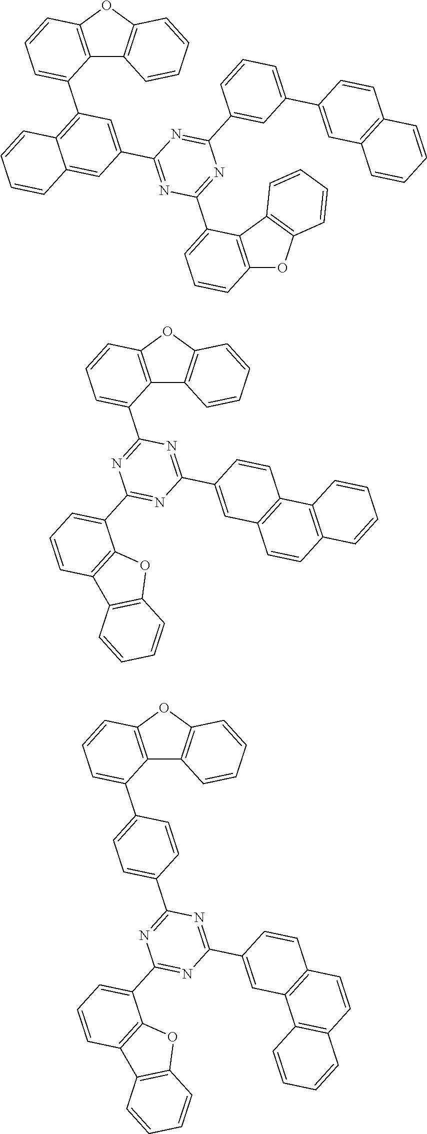 [SMILES: c1cc(-c2ccc3ccccc3c2)cc(-c2nc(-c3cc(-c4cccc5oc6ccccc6c45)c4ccccc4c3)nc(-c3cccc4oc5ccccc5c34)n2)c1 ; c1ccc2c(c1)ccc1cc(-c3nc(-c4cccc5c4oc4ccccc45)nc(-c4cccc5oc6ccccc6c45)n3)ccc12 ; c1ccc2c(c1)ccc1ccc(-c3nc(-c4ccc(-c5cccc6oc7ccccc7c56)cc4)nc(-c4cccc5c4oc4ccccc45)n3)cc12]

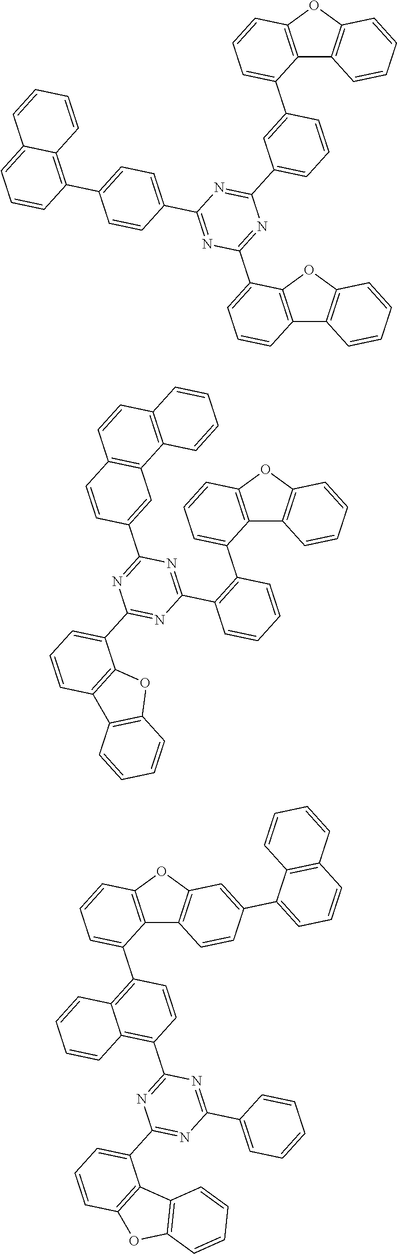 [SMILES: c1cc(-c2nc(-c3ccc(-c4cccc5ccccc45)cc3)nc(-c3cccc4c3oc3ccccc34)n2)cc(-c2cccc3oc4ccccc4c23)c1 ; c1ccc(-c2cccc3oc4ccccc4c23)c(-c2nc(-c3ccc4ccc5ccccc5c4c3)nc(-c3cccc4c3oc3ccccc34)n2)c1 ; c1ccc(-c2nc(-c3ccc(-c4cccc5oc6cc(-c7cccc8ccccc78)ccc6c45)c4ccccc34)nc(-c3cccc4oc5ccccc5c34)n2)cc1]

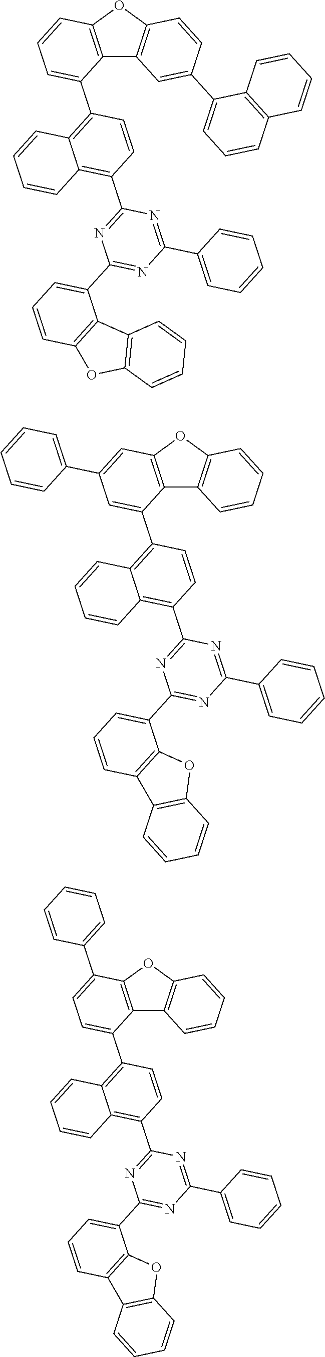 [SMILES: c1ccc(-c2nc(-c3ccc(-c4cccc5oc6ccc(-c7cccc8ccccc78)cc6c45)c4ccccc34)nc(-c3cccc4oc5ccccc5c34)n2)cc1 ; c1ccc(-c2cc(-c3ccc(-c4nc(-c5ccccc5)nc(-c5cccc6c5oc5ccccc56)n4)c4ccccc34)c3c(c2)oc2ccccc23)cc1 ; c1ccc(-c2nc(-c3ccc(-c4ccc(-c5ccccc5)c5oc6ccccc6c45)c4ccccc34)nc(-c3cccc4c3oc3ccccc34)n2)cc1]

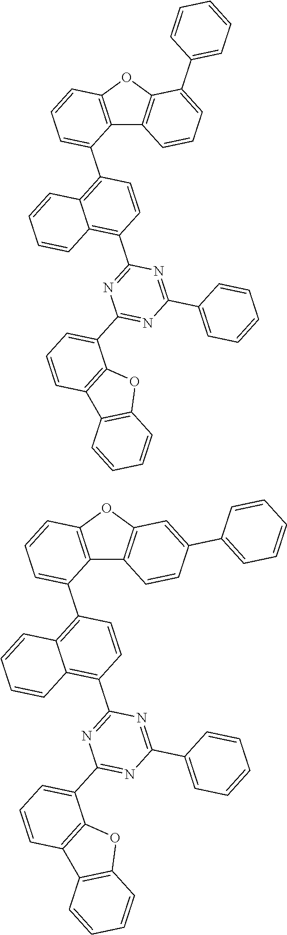

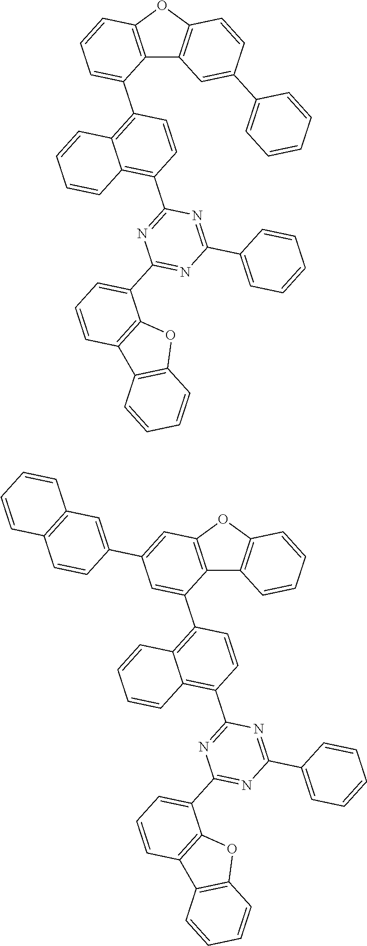 [SMILES: c1ccc(-c2ccc3oc4cccc(-c5ccc(-c6nc(-c7ccccc7)nc(-c7cccc8c7oc7ccccc78)n6)c6ccccc56)c4c3c2)cc1 ; c1ccc(-c2nc(-c3ccc(-c4cc(-c5ccc6ccccc6c5)cc5oc6ccccc6c45)c4ccccc34)nc(-c3cccc4c3oc3ccccc34)n2)cc1]

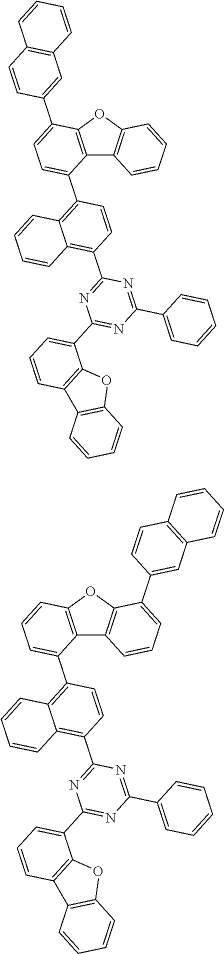 [SMILES: c1ccc(-c2nc(-c3ccc(-c4ccc(-c5ccc6ccccc6c5)c5oc6ccccc6c45)c4ccccc34)nc(-c3cccc4c3oc3ccccc34)n2)cc1 ; c1ccc(-c2nc(-c3ccc(-c4cccc5oc6c(-c7ccc8ccccc8c7)cccc6c45)c4ccccc34)nc(-c3cccc4c3oc3ccccc34)n2)cc1]

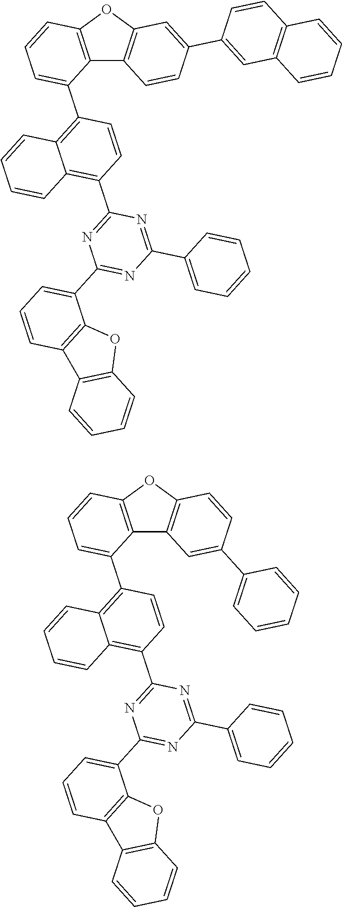 [SMILES: c1ccc(-c2nc(-c3ccc(-c4cccc5oc6cc(-c7ccc8ccccc8c7)ccc6c45)c4ccccc34)nc(-c3cccc4c3oc3ccccc34)n2)cc1 ; c1ccc(-c2ccc3oc4cccc(-c5ccc(-c6nc(-c7ccccc7)nc(-c7cccc8c7oc7ccccc78)n6)c6ccccc56)c4c3c2)cc1]

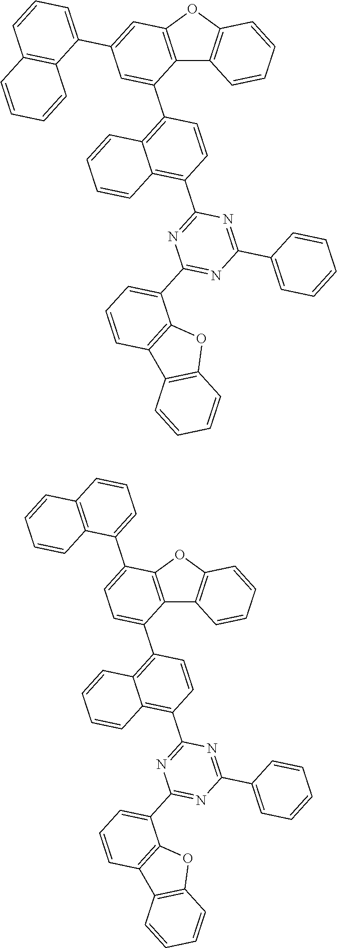 [SMILES: c1ccc(-c2nc(-c3ccc(-c4cc(-c5cccc6ccccc56)cc5oc6ccccc6c45)c4ccccc34)nc(-c3cccc4c3oc3ccccc34)n2)cc1 ; c1ccc(-c2nc(-c3ccc(-c4ccc(-c5cccc6ccccc56)c5oc6ccccc6c45)c4ccccc34)nc(-c3cccc4c3oc3ccccc34)n2)cc1]

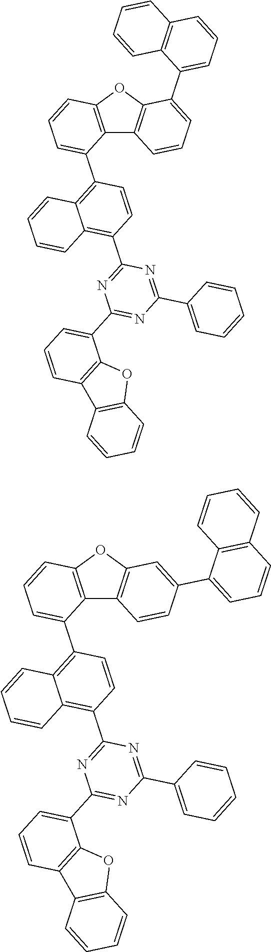 [SMILES: c1ccc(-c2nc(-c3ccc(-c4cccc5oc6c(-c7cccc8ccccc78)cccc6c45)c4ccccc34)nc(-c3cccc4c3oc3ccccc34)n2)cc1 ; c1ccc(-c2nc(-c3ccc(-c4cccc5oc6cc(-c7cccc8ccccc78)ccc6c45)c4ccccc34)nc(-c3cccc4c3oc3ccccc34)n2)cc1]

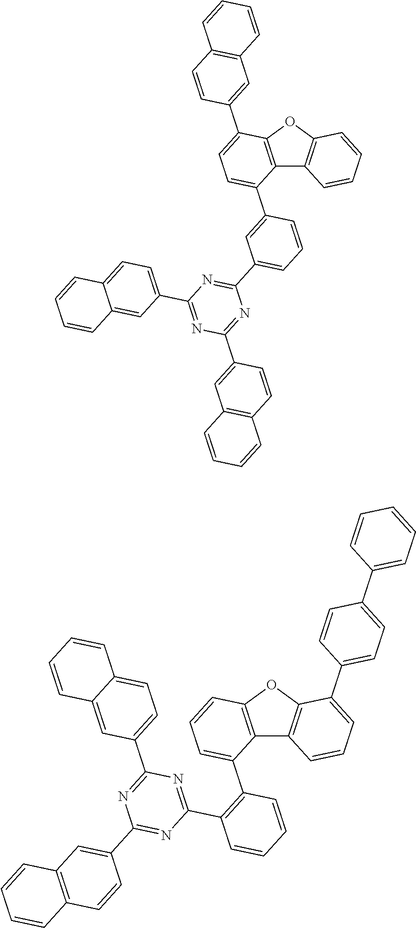 [SMILES: c1cc(-c2nc(-c3ccc4ccccc4c3)nc(-c3ccc4ccccc4c3)n2)cc(-c2ccc(-c3ccc4ccccc4c3)c3oc4ccccc4c23)c1 ; c1ccc(-c2ccc(-c3cccc4c3oc3cccc(-c5ccccc5-c5nc(-c6ccc7ccccc7c6)nc(-c6ccc7ccccc7c6)n5)c34)cc2)cc1]

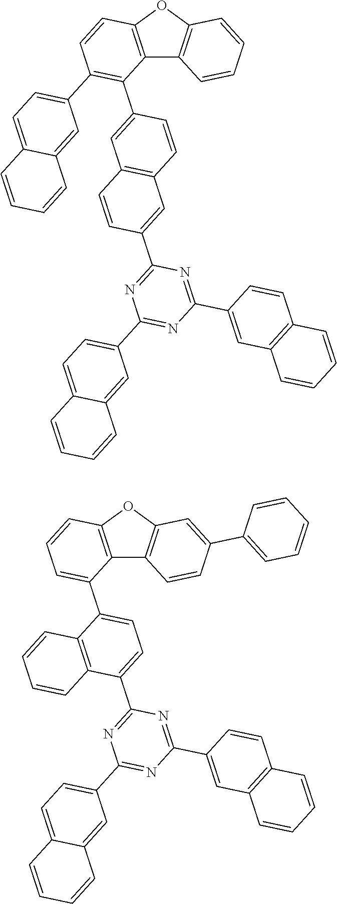 [SMILES: c1ccc2cc(-c3nc(-c4ccc5ccccc5c4)nc(-c4ccc5cc(-c6c(-c7ccc8ccccc8c7)ccc7oc8ccccc8c67)ccc5c4)n3)ccc2c1 ; c1ccc(-c2ccc3c(c2)oc2cccc(-c4ccc(-c5nc(-c6ccc7ccccc7c6)nc(-c6ccc7ccccc7c6)n5)c5ccccc45)c23)cc1]

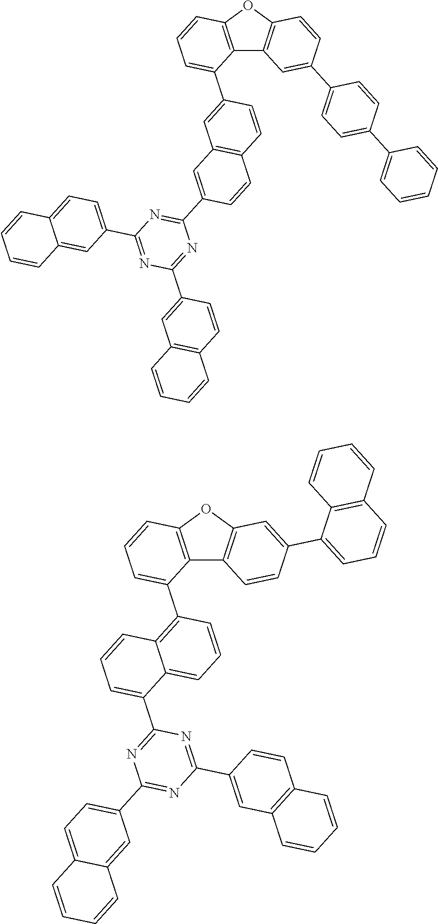 [SMILES: c1ccc(-c2ccc(-c3ccc4oc5cccc(-c6ccc7ccc(-c8nc(-c9ccc%10ccccc%10c9)nc(-c9ccc%10ccccc%10c9)n8)cc7c6)c5c4c3)cc2)cc1 ; c1ccc2cc(-c3nc(-c4ccc5ccccc5c4)nc(-c4cccc5c(-c6cccc7oc8cc(-c9cccc%10ccccc9%10)ccc8c67)cccc45)n3)ccc2c1]

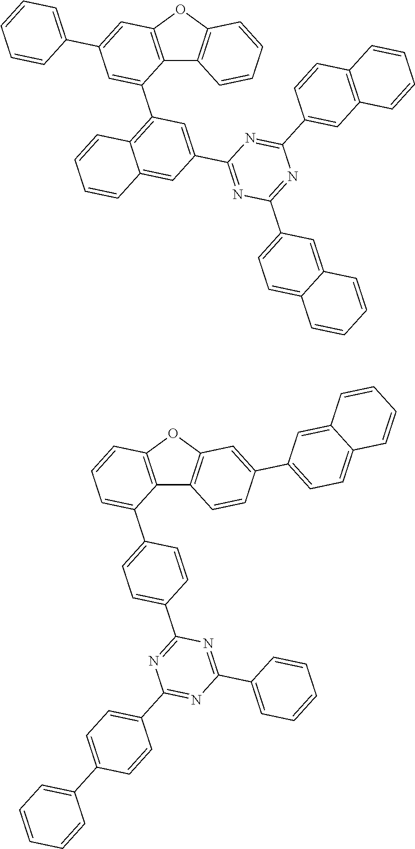 [SMILES: c1ccc(-c2cc(-c3cc(-c4nc(-c5ccc6ccccc6c5)nc(-c5ccc6ccccc6c5)n4)cc4ccccc34)c3c(c2)oc2ccccc23)cc1 ; c1ccc(-c2ccc(-c3nc(-c4ccccc4)nc(-c4ccc(-c5cccc6oc7cc(-c8ccc9ccccc9c8)ccc7c56)cc4)n3)cc2)cc1]

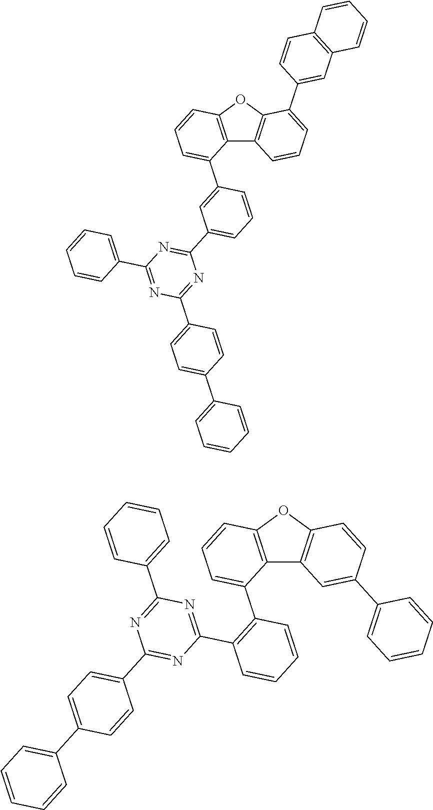 [SMILES: c1ccc(-c2ccc(-c3nc(-c4ccccc4)nc(-c4cccc(-c5cccc6oc7c(-c8ccc9ccccc9c8)cccc7c56)c4)n3)cc2)cc1 ; c1ccc(-c2ccc(-c3nc(-c4ccccc4)nc(-c4ccccc4-c4cccc5oc6ccc(-c7ccccc7)cc6c45)n3)cc2)cc1]

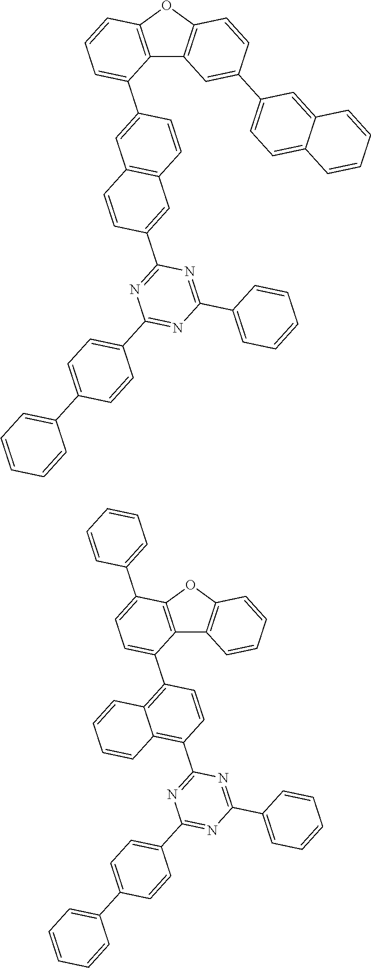 [SMILES: c1ccc(-c2ccc(-c3nc(-c4ccccc4)nc(-c4ccc5cc(-c6cccc7oc8ccc(-c9ccc%10ccccc%10c9)cc8c67)ccc5c4)n3)cc2)cc1 ; c1ccc(-c2ccc(-c3nc(-c4ccccc4)nc(-c4ccc(-c5ccc(-c6ccccc6)c6oc7ccccc7c56)c5ccccc45)n3)cc2)cc1]

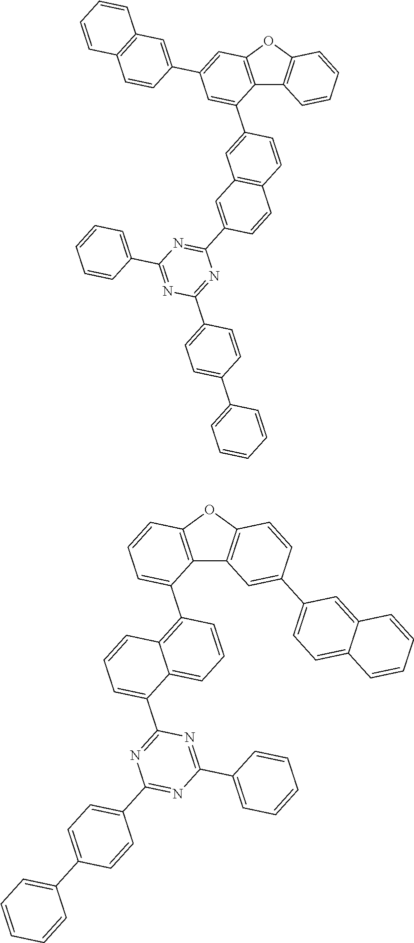 [SMILES: c1ccc(-c2ccc(-c3nc(-c4ccccc4)nc(-c4ccc5ccc(-c6cc(-c7ccc8ccccc8c7)cc7oc8ccccc8c67)cc5c4)n3)cc2)cc1 ; c1ccc(-c2ccc(-c3nc(-c4ccccc4)nc(-c4cccc5c(-c6cccc7oc8ccc(-c9ccc%10ccccc%10c9)cc8c67)cccc45)n3)cc2)cc1]

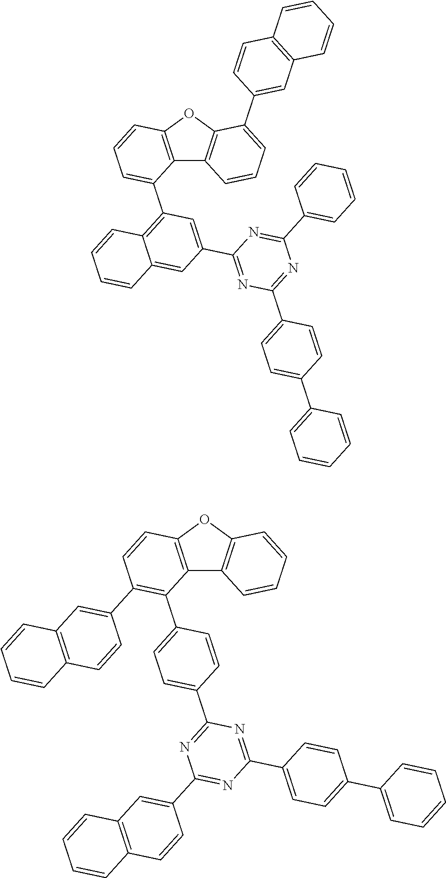 [SMILES: c1ccc(-c2ccc(-c3nc(-c4ccccc4)nc(-c4cc(-c5cccc6oc7c(-c8ccc9ccccc9c8)cccc7c56)c5ccccc5c4)n3)cc2)cc1 ; c1ccc(-c2ccc(-c3nc(-c4ccc(-c5c(-c6ccc7ccccc7c6)ccc6oc7ccccc7c56)cc4)nc(-c4ccc5ccccc5c4)n3)cc2)cc1]

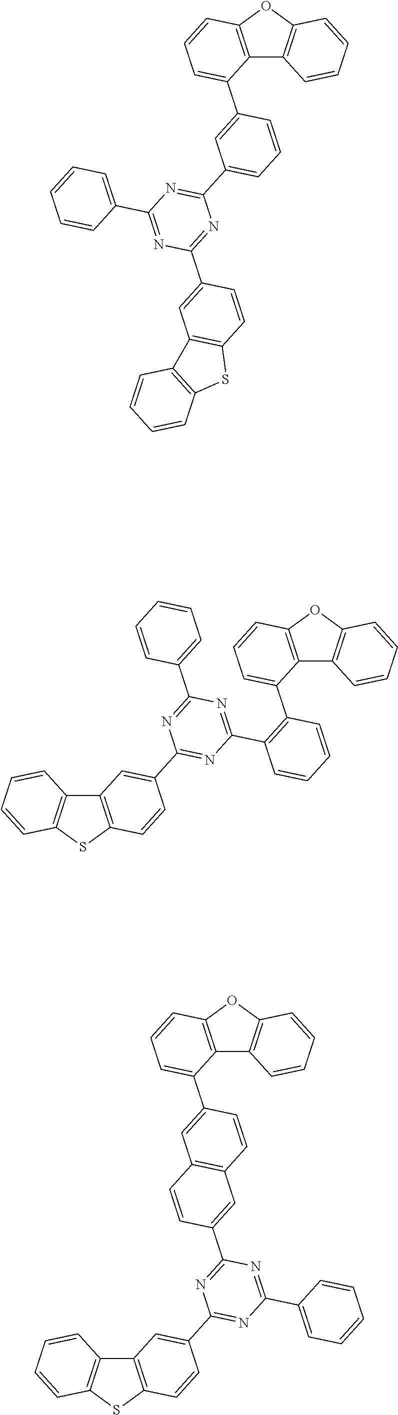 [SMILES: c1ccc(-c2nc(-c3cccc(-c4cccc5oc6ccccc6c45)c3)nc(-c3ccc4sc5ccccc5c4c3)n2)cc1 ; c1ccc(-c2nc(-c3ccc4sc5ccccc5c4c3)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4sc5ccccc5c4c3)n2)cc1]

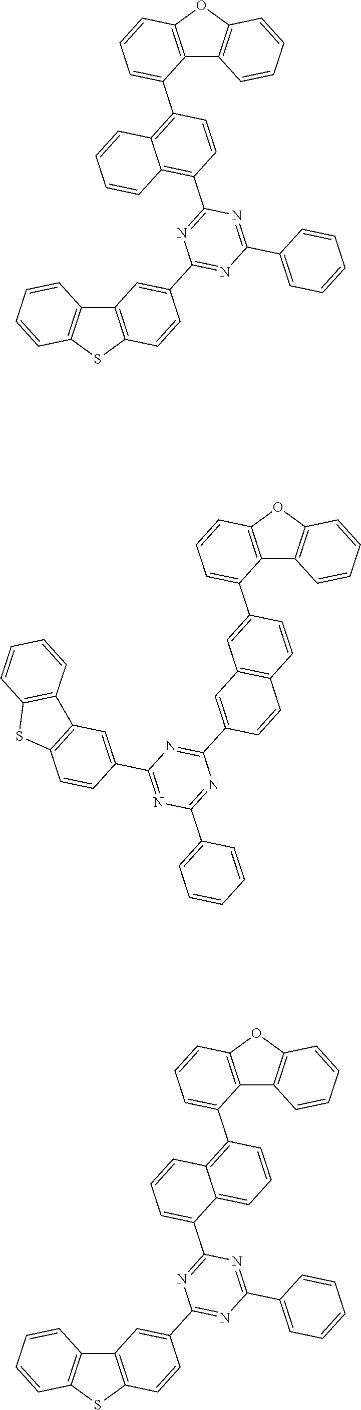 [SMILES: c1ccc(-c2nc(-c3ccc4sc5ccccc5c4c3)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4sc5ccccc5c4c3)n2)cc1 ; c1ccc(-c2nc(-c3ccc4sc5ccccc5c4c3)nc(-c3cccc4c(-c5cccc6oc7ccccc7c56)cccc34)n2)cc1]

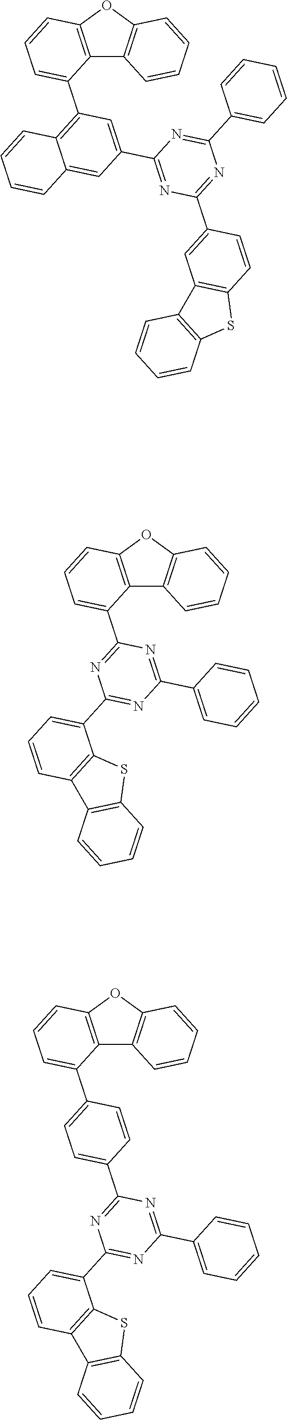 [SMILES: c1ccc(-c2nc(-c3cc(-c4cccc5oc6ccccc6c45)c4ccccc4c3)nc(-c3ccc4sc5ccccc5c4c3)n2)cc1 ; c1ccc(-c2nc(-c3cccc4c3sc3ccccc34)nc(-c3cccc4oc5ccccc5c34)n2)cc1 ; c1ccc(-c2nc(-c3ccc(-c4cccc5oc6ccccc6c45)cc3)nc(-c3cccc4c3sc3ccccc34)n2)cc1]

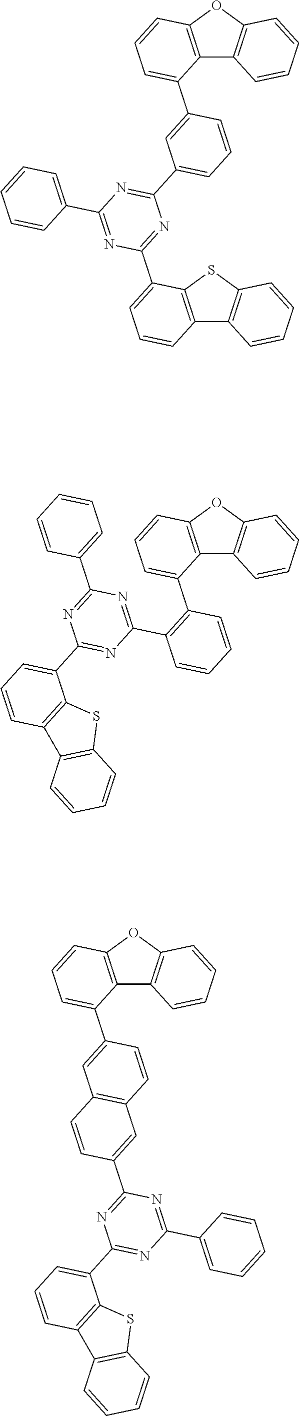 [SMILES: c1ccc(-c2nc(-c3cccc(-c4cccc5oc6ccccc6c45)c3)nc(-c3cccc4c3sc3ccccc34)n2)cc1 ; c1ccc(-c2nc(-c3ccccc3-c3cccc4oc5ccccc5c34)nc(-c3cccc4c3sc3ccccc34)n2)cc1 ; c1ccc(-c2nc(-c3ccc4cc(-c5cccc6oc7ccccc7c56)ccc4c3)nc(-c3cccc4c3sc3ccccc34)n2)cc1]

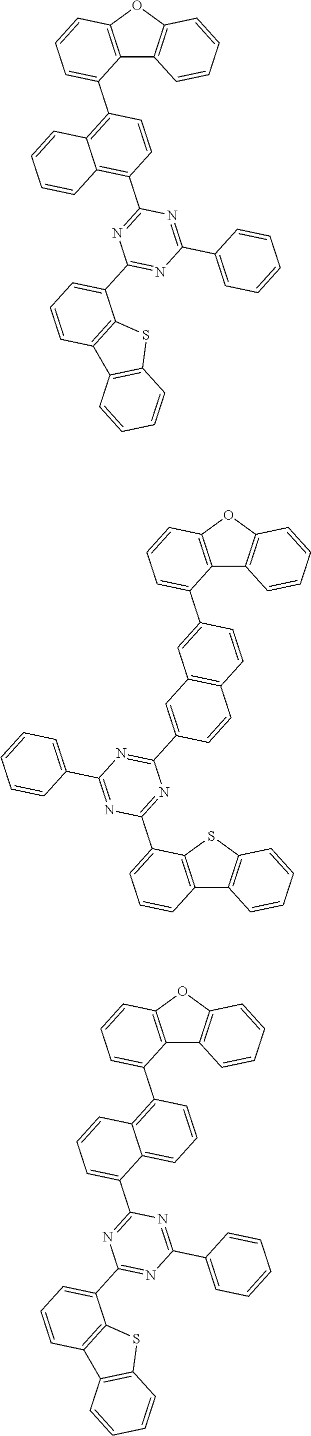 [SMILES: c1ccc(-c2nc(-c3ccc(-c4cccc5oc6ccccc6c45)c4ccccc34)nc(-c3cccc4c3sc3ccccc34)n2)cc1 ; c1ccc(-c2nc(-c3ccc4ccc(-c5cccc6oc7ccccc7c56)cc4c3)nc(-c3cccc4c3sc3ccccc34)n2)cc1 ; c1ccc(-c2nc(-c3cccc4c(-c5cccc6oc7ccccc7c56)cccc34)nc(-c3cccc4c3sc3ccccc34)n2)cc1]

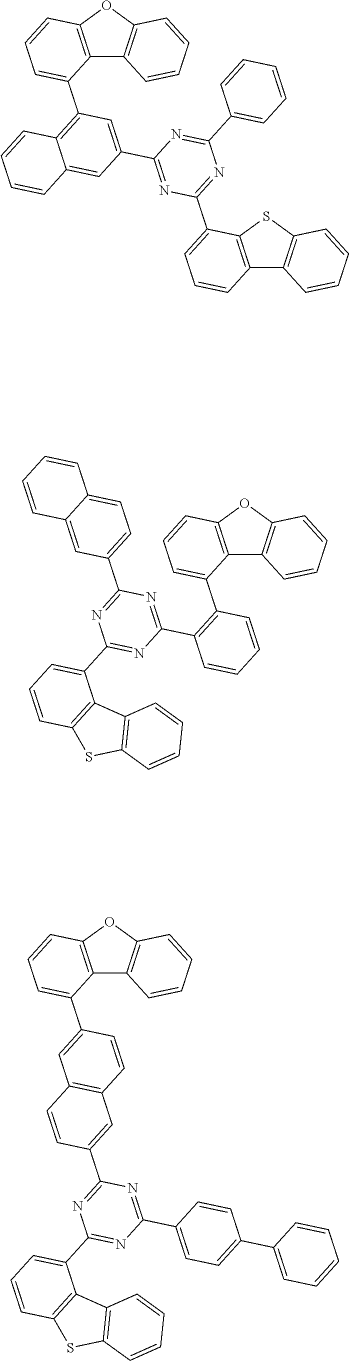 [SMILES: c1ccc(-c2nc(-c3cc(-c4cccc5oc6ccccc6c45)c4ccccc4c3)nc(-c3cccc4c3sc3ccccc34)n2)cc1 ; c1ccc(-c2cccc3oc4ccccc4c23)c(-c2nc(-c3ccc4ccccc4c3)nc(-c3cccc4sc5ccccc5c34)n2)c1 ; c1ccc(-c2ccc(-c3nc(-c4ccc5cc(-c6cccc7oc8ccccc8c67)ccc5c4)nc(-c4cccc5sc6ccccc6c45)n3)cc2)cc1]

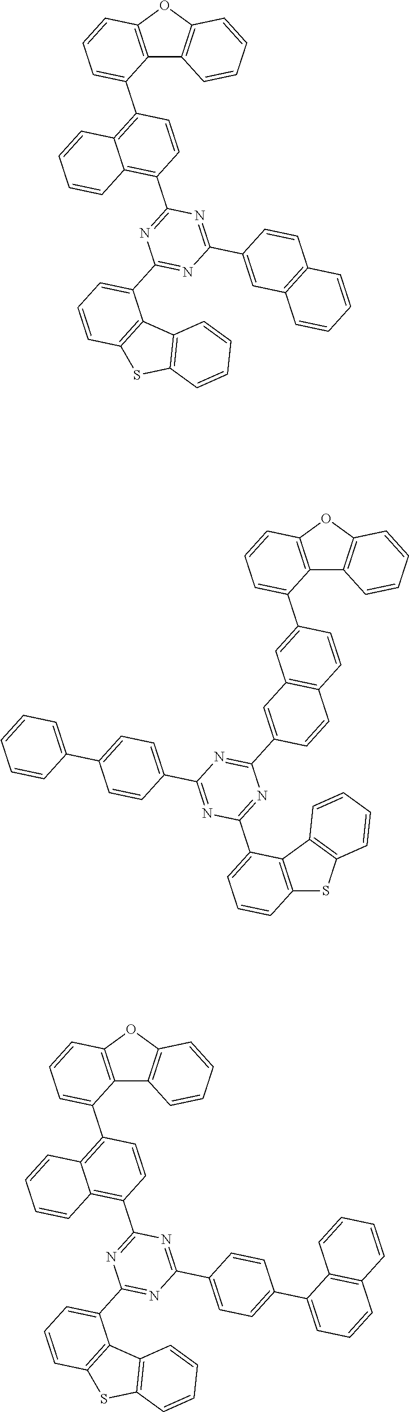

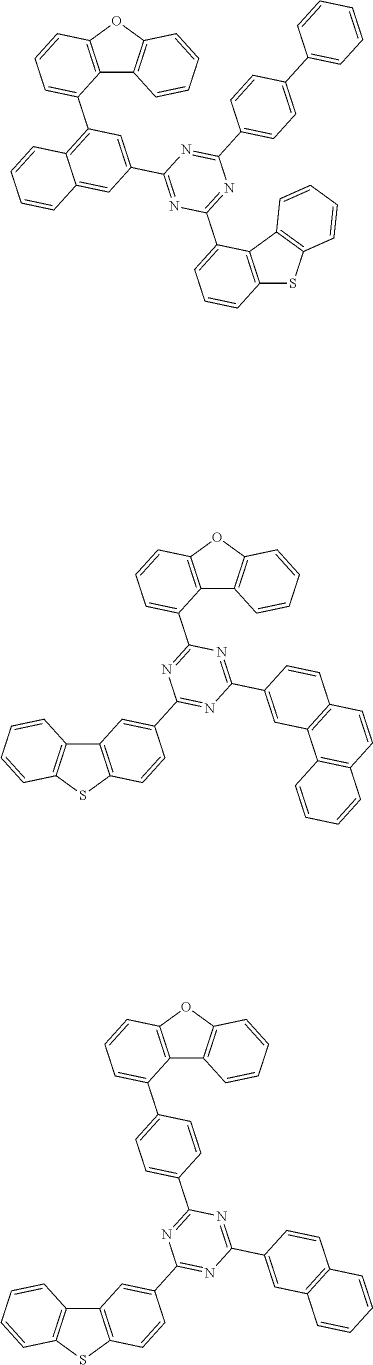 [SMILES: c1ccc(-c2ccc(-c3nc(-c4cc(-c5cccc6oc7ccccc7c56)c5ccccc5c4)nc(-c4cccc5sc6ccccc6c45)n3)cc2)cc1 ; c1ccc2c(c1)ccc1ccc(-c3nc(-c4ccc5sc6ccccc6c5c4)nc(-c4cccc5oc6ccccc6c45)n3)cc12 ; c1ccc2cc(-c3nc(-c4ccc(-c5cccc6oc7ccccc7c56)cc4)nc(-c4ccc5sc6ccccc6c5c4)n3)ccc2c1]

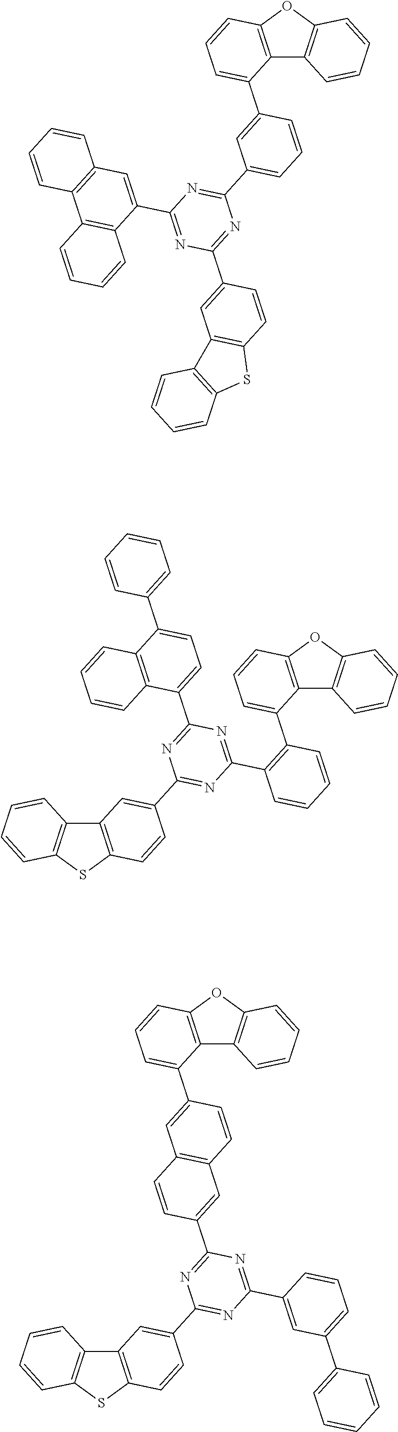 [SMILES: c1cc(-c2nc(-c3ccc4sc5ccccc5c4c3)nc(-c3cc4ccccc4c4ccccc34)n2)cc(-c2cccc3oc4ccccc4c23)c1 ; c1ccc(-c2ccc(-c3nc(-c4ccc5sc6ccccc6c5c4)nc(-c4ccccc4-c4cccc5oc6ccccc6c45)n3)c3ccccc23)cc1 ; c1ccc(-c2cccc(-c3nc(-c4ccc5cc(-c6cccc7oc8ccccc8c67)ccc5c4)nc(-c4ccc5sc6ccccc6c5c4)n3)c2)cc1]

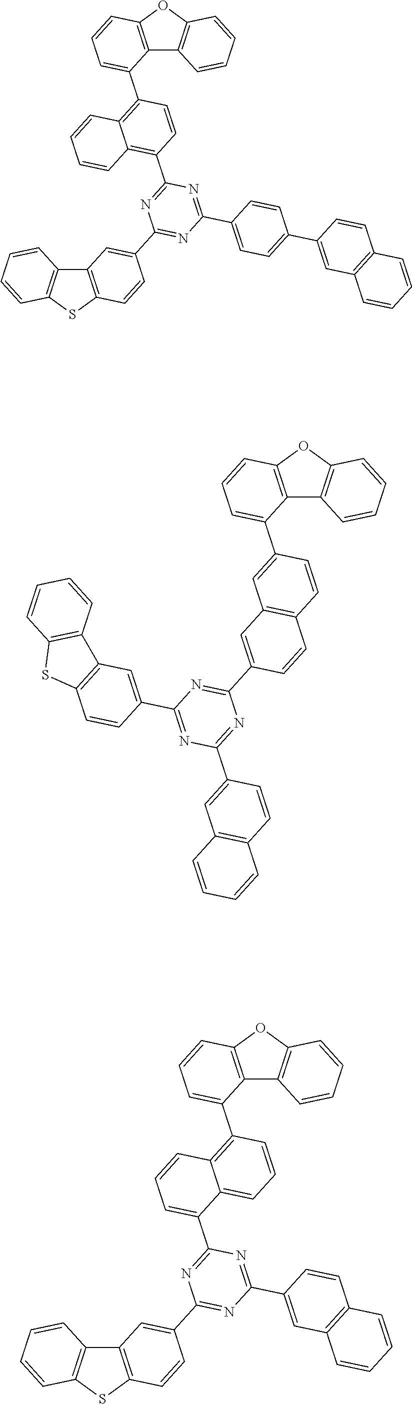 [SMILES: c1ccc2cc(-c3ccc(-c4nc(-c5ccc6sc7ccccc7c6c5)nc(-c5ccc(-c6cccc7oc8ccccc8c67)c6ccccc56)n4)cc3)ccc2c1 ; c1ccc2cc(-c3nc(-c4ccc5ccc(-c6cccc7oc8ccccc8c67)cc5c4)nc(-c4ccc5sc6ccccc6c5c4)n3)ccc2c1 ; c1ccc2cc(-c3nc(-c4ccc5sc6ccccc6c5c4)nc(-c4cccc5c(-c6cccc7oc8ccccc8c67)cccc45)n3)ccc2c1]

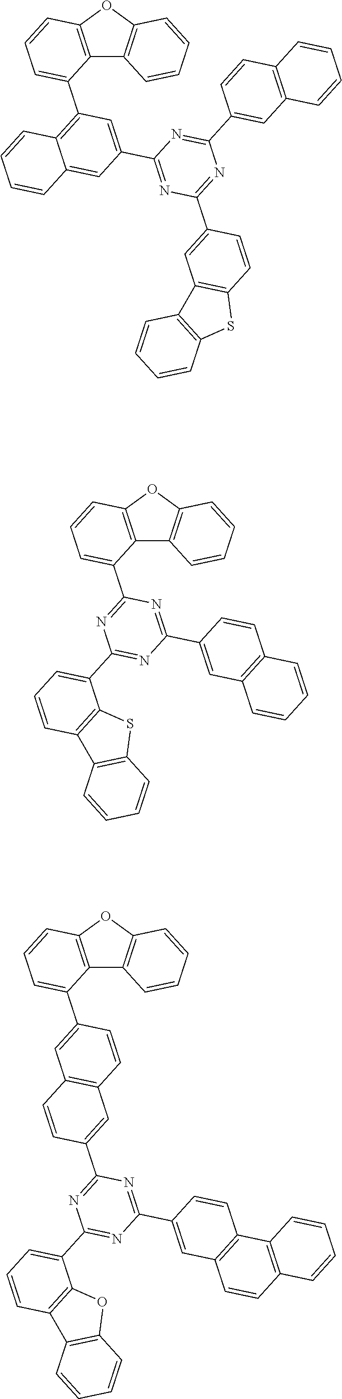 [SMILES: c1ccc2cc(-c3nc(-c4cc(-c5cccc6oc7ccccc7c56)c5ccccc5c4)nc(-c4ccc5sc6ccccc6c5c4)n3)ccc2c1 ; c1ccc2cc(-c3nc(-c4cccc5c4sc4ccccc45)nc(-c4cccc5oc6ccccc6c45)n3)ccc2c1 ; c1ccc2c(c1)ccc1cc(-c3nc(-c4ccc5cc(-c6cccc7oc8ccccc8c67)ccc5c4)nc(-c4cccc5c4oc4ccccc45)n3)ccc12]

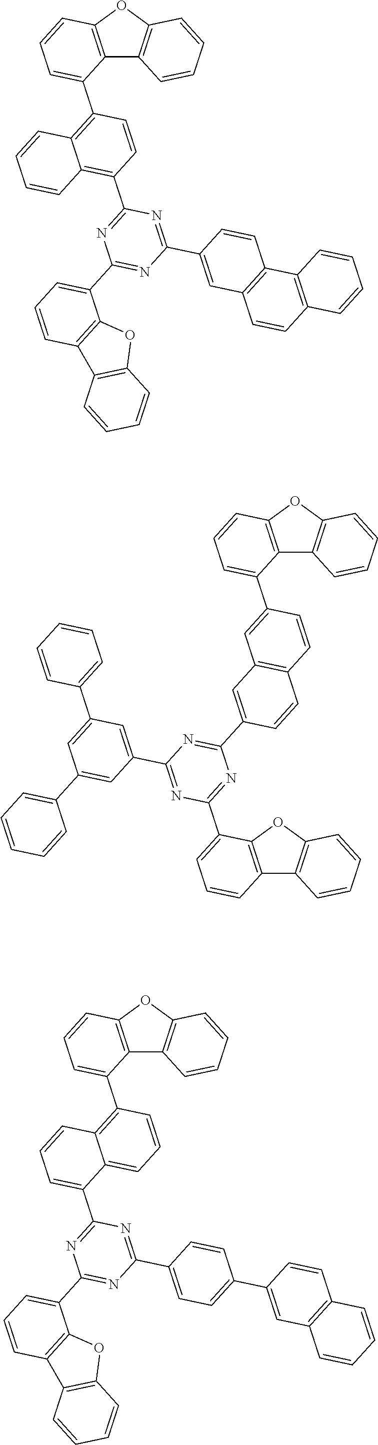 [SMILES: c1ccc2c(c1)ccc1cc(-c3nc(-c4ccc(-c5cccc6oc7ccccc7c56)c5ccccc45)nc(-c4cccc5c4oc4ccccc45)n3)ccc12 ; c1ccc(-c2cc(-c3ccccc3)cc(-c3nc(-c4ccc5ccc(-c6cccc7oc8ccccc8c67)cc5c4)nc(-c4cccc5c4oc4ccccc45)n3)c2)cc1 ; c1ccc2cc(-c3ccc(-c4nc(-c5cccc6c(-c7cccc8oc9ccccc9c78)cccc56)nc(-c5cccc6c5oc5ccccc56)n4)cc3)ccc2c1]

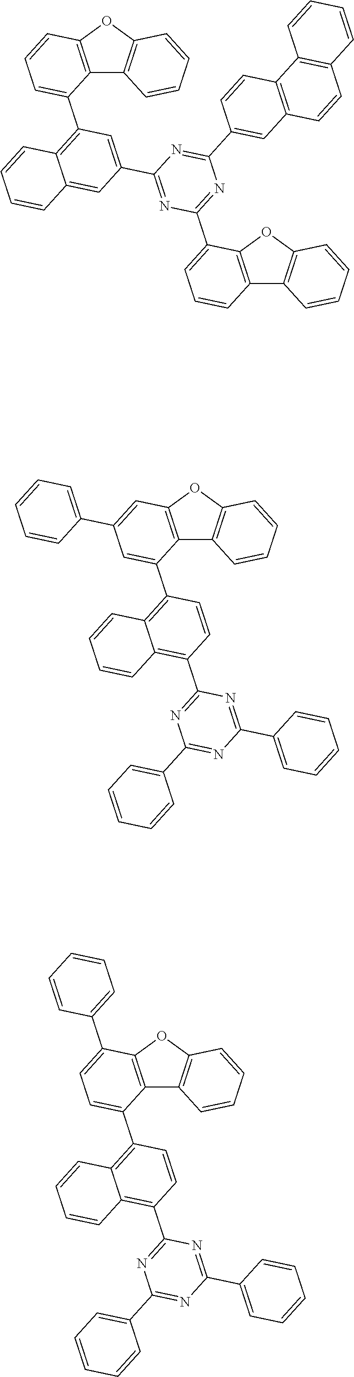 [SMILES: c1ccc2c(-c3cccc4oc5ccccc5c34)cc(-c3nc(-c4ccc5c(ccc6ccccc65)c4)nc(-c4cccc5c4oc4ccccc45)n3)cc2c1 ; c1ccc(-c2cc(-c3ccc(-c4nc(-c5ccccc5)nc(-c5ccccc5)n4)c4ccccc34)c3c(c2)oc2ccccc23)cc1 ; c1ccc(-c2nc(-c3ccccc3)nc(-c3ccc(-c4ccc(-c5ccccc5)c5oc6ccccc6c45)c4ccccc34)n2)cc1]

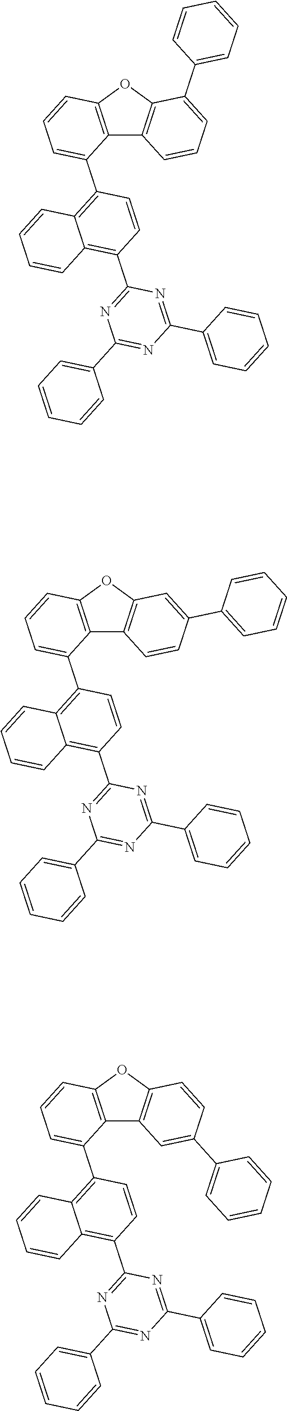 [SMILES: c1ccc(-c2nc(-c3ccccc3)nc(-c3ccc(-c4cccc5oc6c(-c7ccccc7)cccc6c45)c4ccccc34)n2)cc1 ; c1ccc(-c2ccc3c(c2)oc2cccc(-c4ccc(-c5nc(-c6ccccc6)nc(-c6ccccc6)n5)c5ccccc45)c23)cc1 ; c1ccc(-c2ccc3oc4cccc(-c5ccc(-c6nc(-c7ccccc7)nc(-c7ccccc7)n6)c6ccccc56)c4c3c2)cc1]

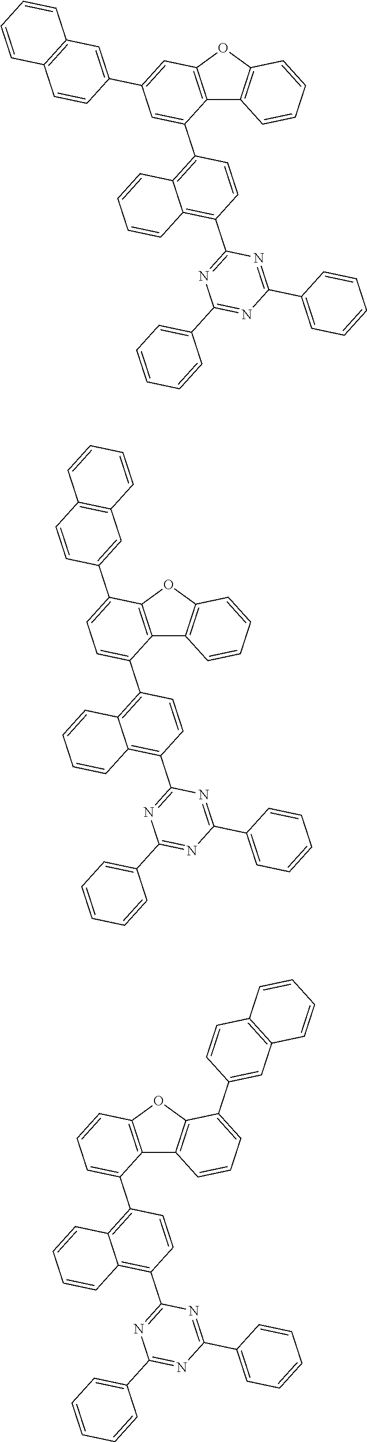 [SMILES: c1ccc(-c2nc(-c3ccccc3)nc(-c3ccc(-c4cc(-c5ccc6ccccc6c5)cc5oc6ccccc6c45)c4ccccc34)n2)cc1 ; c1ccc(-c2nc(-c3ccccc3)nc(-c3ccc(-c4ccc(-c5ccc6ccccc6c5)c5oc6ccccc6c45)c4ccccc34)n2)cc1 ; c1ccc(-c2nc(-c3ccccc3)nc(-c3ccc(-c4cccc5oc6c(-c7ccc8ccccc8c7)cccc6c45)c4ccccc34)n2)cc1]

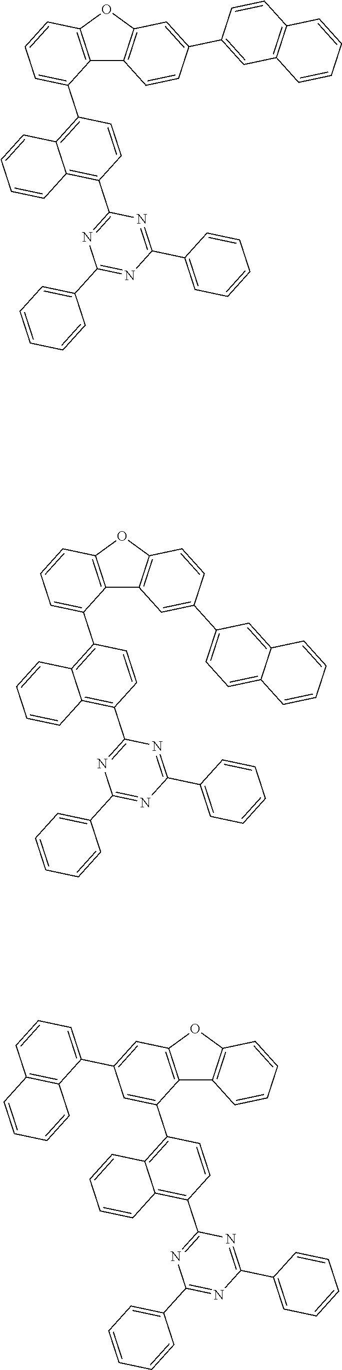 [SMILES: c1ccc(-c2nc(-c3ccccc3)nc(-c3ccc(-c4cccc5oc6cc(-c7ccc8ccccc8c7)ccc6c45)c4ccccc34)n2)cc1 ; c1ccc(-c2nc(-c3ccccc3)nc(-c3ccc(-c4cccc5oc6ccc(-c7ccc8ccccc8c7)cc6c45)c4ccccc34)n2)cc1 ; c1ccc(-c2nc(-c3ccccc3)nc(-c3ccc(-c4cc(-c5cccc6ccccc56)cc5oc6ccccc6c45)c4ccccc34)n2)cc1]

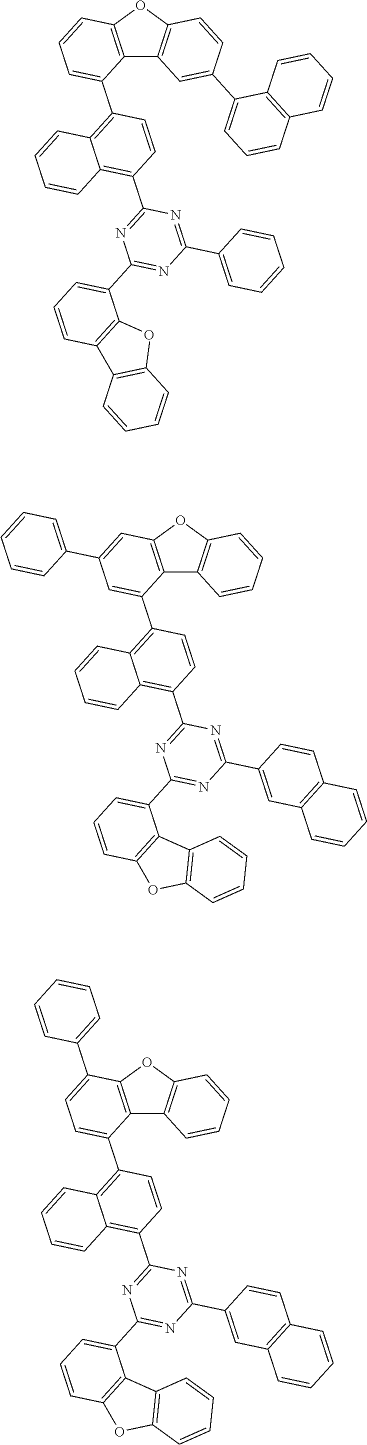 [SMILES: c1ccc(-c2nc(-c3ccc(-c4cccc5oc6ccc(-c7cccc8ccccc78)cc6c45)c4ccccc34)nc(-c3cccc4c3oc3ccccc34)n2)cc1 ; c1ccc(-c2cc(-c3ccc(-c4nc(-c5ccc6ccccc6c5)nc(-c5cccc6oc7ccccc7c56)n4)c4ccccc34)c3c(c2)oc2ccccc23)cc1 ; c1ccc(-c2ccc(-c3ccc(-c4nc(-c5ccc6ccccc6c5)nc(-c5cccc6oc7ccccc7c56)n4)c4ccccc34)c3c2oc2ccccc23)cc1]

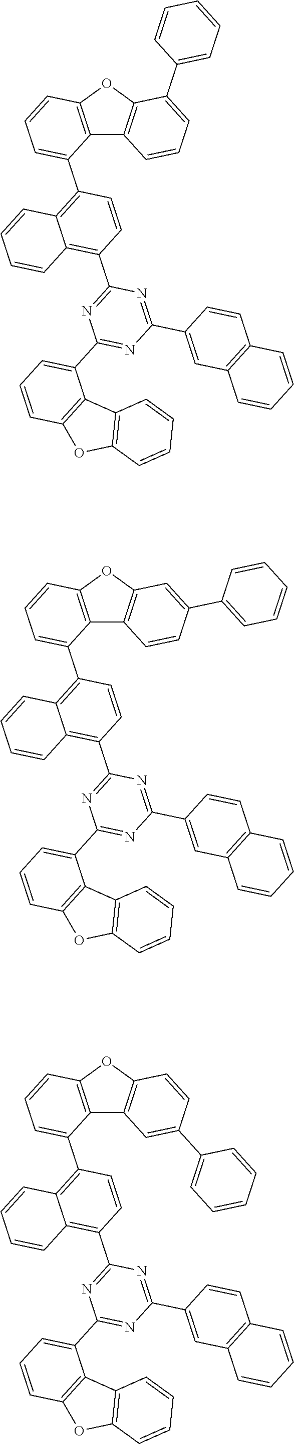 [SMILES: c1ccc(-c2cccc3c2oc2cccc(-c4ccc(-c5nc(-c6ccc7ccccc7c6)nc(-c6cccc7oc8ccccc8c67)n5)c5ccccc45)c23)cc1 ; c1ccc(-c2ccc3c(c2)oc2cccc(-c4ccc(-c5nc(-c6ccc7ccccc7c6)nc(-c6cccc7oc8ccccc8c67)n5)c5ccccc45)c23)cc1 ; c1ccc(-c2ccc3oc4cccc(-c5ccc(-c6nc(-c7ccc8ccccc8c7)nc(-c7cccc8oc9ccccc9c78)n6)c6ccccc56)c4c3c2)cc1]

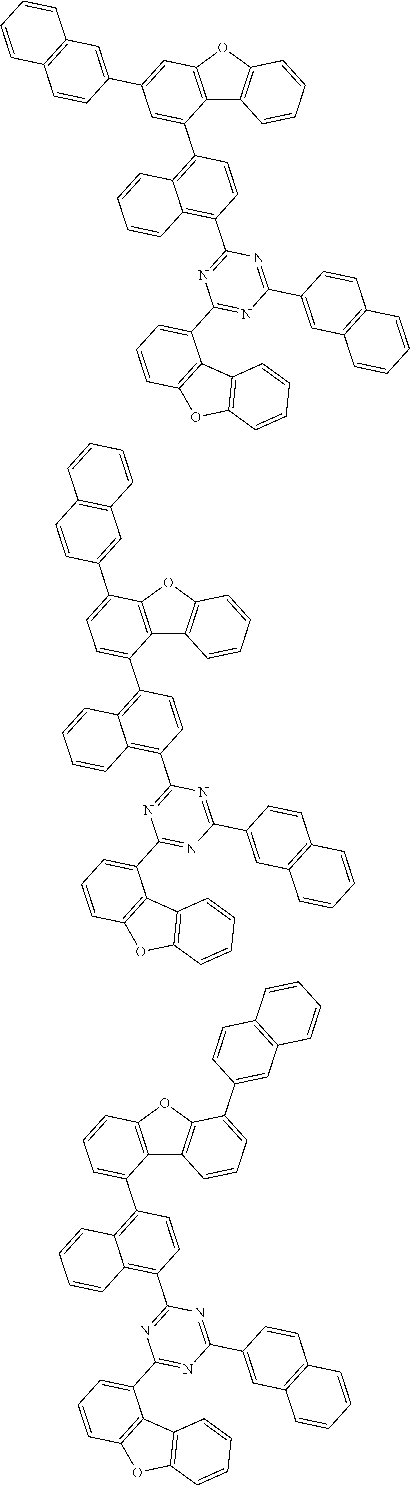 [SMILES: c1ccc2cc(-c3cc(-c4ccc(-c5nc(-c6ccc7ccccc7c6)nc(-c6cccc7oc8ccccc8c67)n5)c5ccccc45)c4c(c3)oc3ccccc34)ccc2c1 ; c1ccc2cc(-c3nc(-c4ccc(-c5ccc(-c6ccc7ccccc7c6)c6oc7ccccc7c56)c5ccccc45)nc(-c4cccc5oc6ccccc6c45)n3)ccc2c1 ; c1ccc2cc(-c3nc(-c4ccc(-c5cccc6oc7c(-c8ccc9ccccc9c8)cccc7c56)c5ccccc45)nc(-c4cccc5oc6ccccc6c45)n3)ccc2c1]

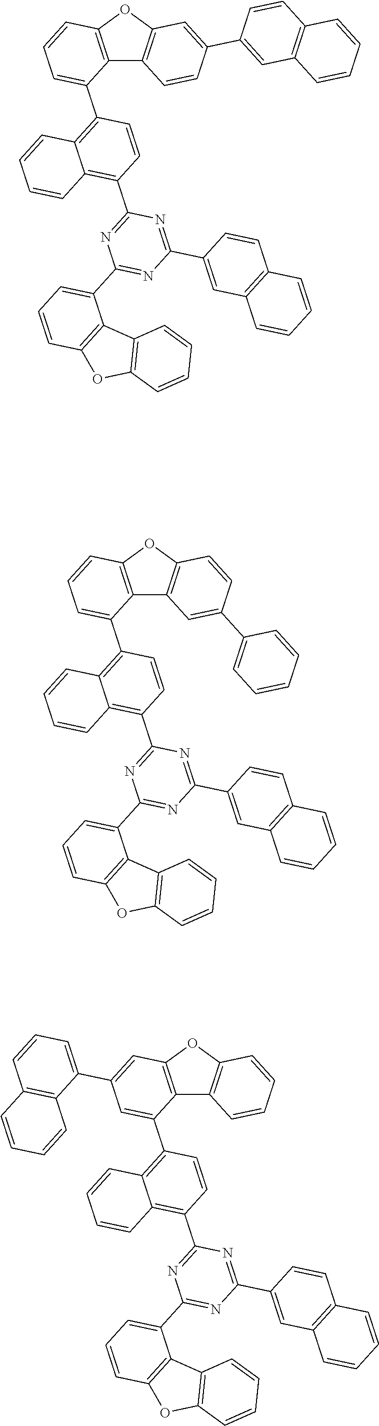 [SMILES: c1ccc2cc(-c3ccc4c(c3)oc3cccc(-c5ccc(-c6nc(-c7ccc8ccccc8c7)nc(-c7cccc8oc9ccccc9c78)n6)c6ccccc56)c34)ccc2c1 ; c1ccc(-c2ccc3oc4cccc(-c5ccc(-c6nc(-c7ccc8ccccc8c7)nc(-c7cccc8oc9ccccc9c78)n6)c6ccccc56)c4c3c2)cc1 ; c1ccc2cc(-c3nc(-c4ccc(-c5cc(-c6cccc7ccccc67)cc6oc7ccccc7c56)c5ccccc45)nc(-c4cccc5oc6ccccc6c45)n3)ccc2c1]

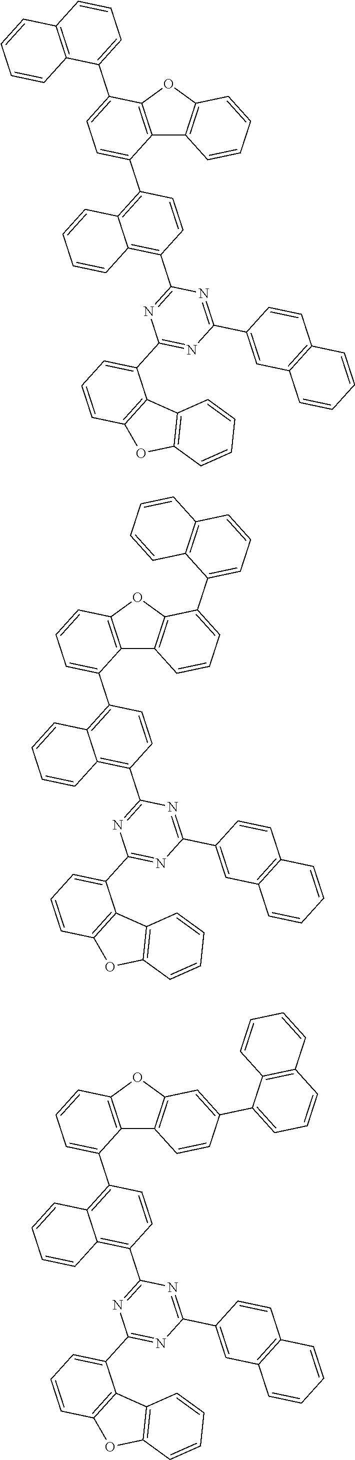

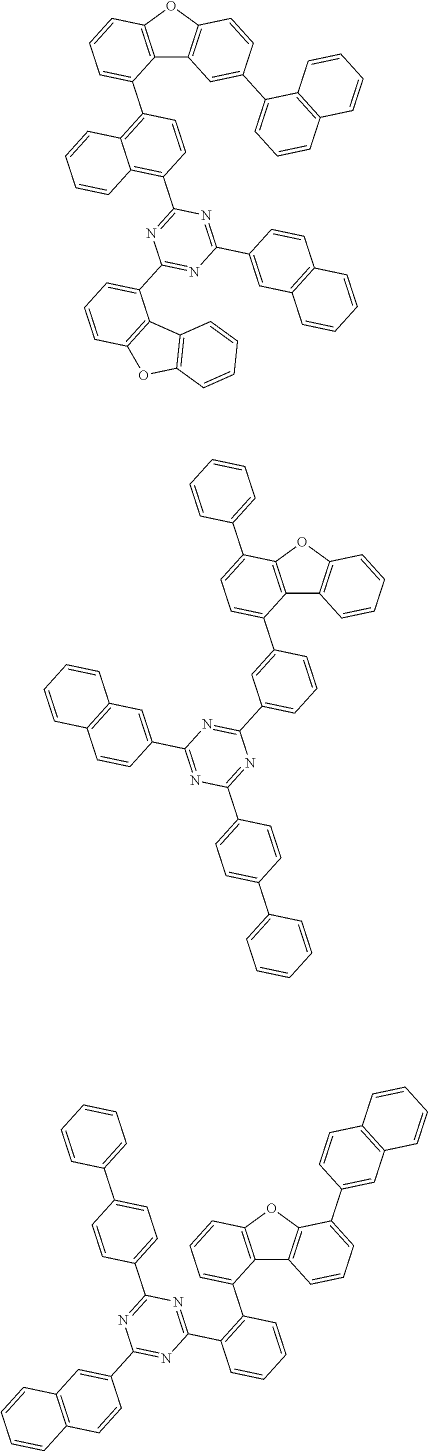 [SMILES: c1ccc2cc(-c3nc(-c4ccc(-c5cccc6oc7ccc(-c8cccc9ccccc89)cc7c56)c5ccccc45)nc(-c4cccc5oc6ccccc6c45)n3)ccc2c1 ; c1ccc(-c2ccc(-c3nc(-c4cccc(-c5ccc(-c6ccccc6)c6oc7ccccc7c56)c4)nc(-c4ccc5ccccc5c4)n3)cc2)cc1 ; c1ccc(-c2ccc(-c3nc(-c4ccc5ccccc5c4)nc(-c4ccccc4-c4cccc5oc6c(-c7ccc8ccccc8c7)cccc6c45)n3)cc2)cc1]

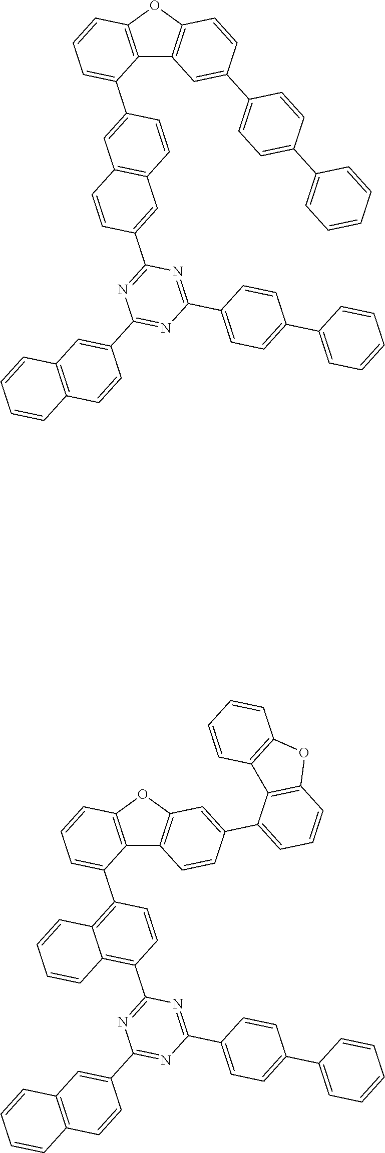 [SMILES: c1ccc(-c2ccc(-c3ccc4oc5cccc(-c6ccc7cc(-c8nc(-c9ccc(-c%10ccccc%10)cc9)nc(-c9ccc%10ccccc%10c9)n8)ccc7c6)c5c4c3)cc2)cc1 ; c1ccc(-c2ccc(-c3nc(-c4ccc5ccccc5c4)nc(-c4ccc(-c5cccc6oc7cc(-c8cccc9oc%10ccccc%10c89)ccc7c56)c5ccccc45)n3)cc2)cc1]

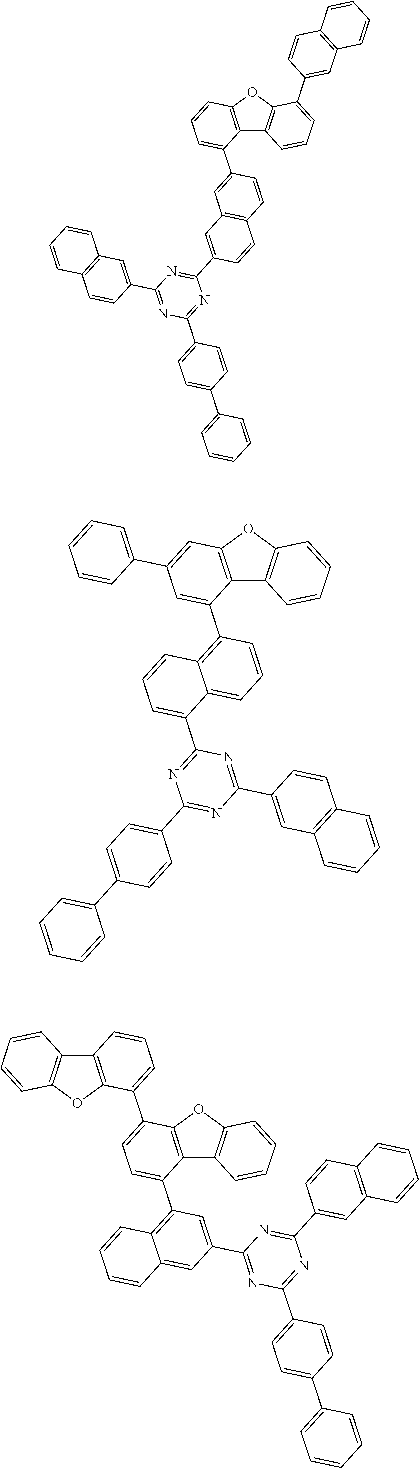 [SMILES: c1ccc(-c2ccc(-c3nc(-c4ccc5ccccc5c4)nc(-c4ccc5ccc(-c6cccc7oc8c(-c9ccc%10ccccc%10c9)cccc8c67)cc5c4)n3)cc2)cc1 ; c1ccc(-c2ccc(-c3nc(-c4ccc5ccccc5c4)nc(-c4cccc5c(-c6cc(-c7ccccc7)cc7oc8ccccc8c67)cccc45)n3)cc2)cc1 ; c1ccc(-c2ccc(-c3nc(-c4ccc5ccccc5c4)nc(-c4cc(-c5ccc(-c6cccc7c6oc6ccccc67)c6oc7ccccc7c56)c5ccccc5c4)n3)cc2)cc1]

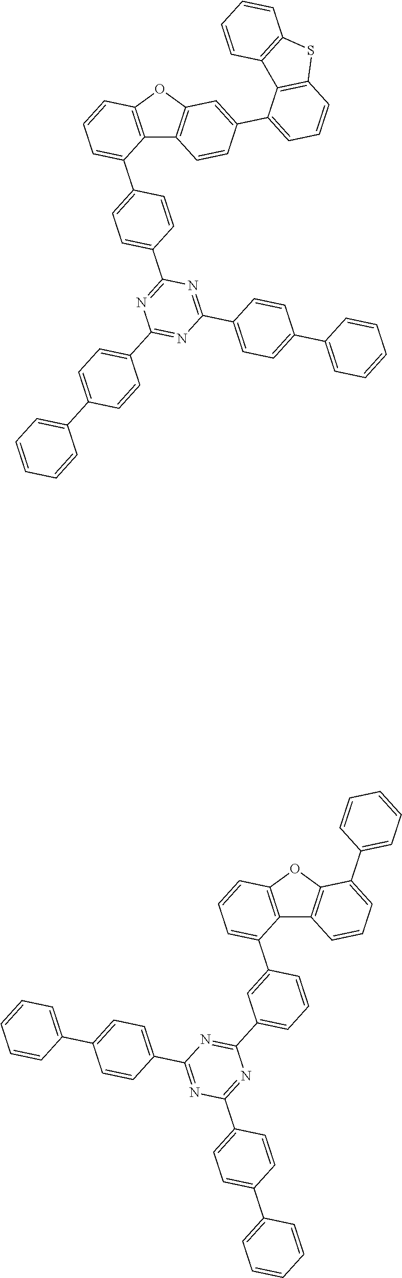 [SMILES: c1ccc(-c2ccc(-c3nc(-c4ccc(-c5ccccc5)cc4)nc(-c4ccc(-c5cccc6oc7cc(-c8cccc9sc%10ccccc%10c89)ccc7c56)cc4)n3)cc2)cc1 ; c1ccc(-c2ccc(-c3nc(-c4ccc(-c5ccccc5)cc4)nc(-c4cccc(-c5cccc6oc7c(-c8ccccc8)cccc7c56)c4)n3)cc2)cc1]

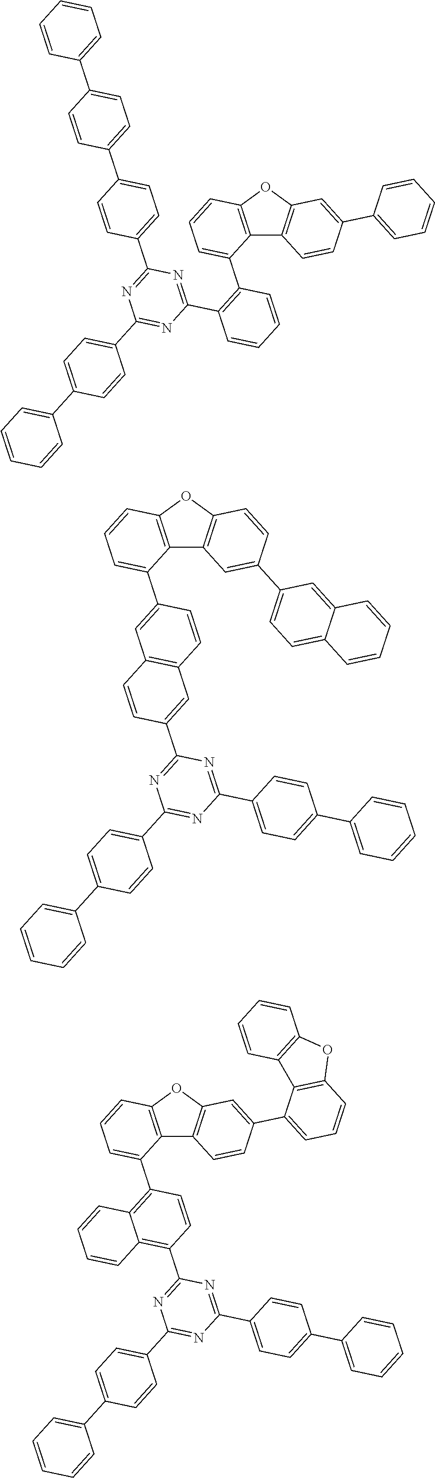 [SMILES: c1ccc(-c2ccc(-c3ccc(-c4nc(-c5ccc(-c6ccccc6)cc5)nc(-c5ccccc5-c5cccc6oc7cc(-c8ccccc8)ccc7c56)n4)cc3)cc2)cc1 ; c1ccc(-c2ccc(-c3nc(-c4ccc(-c5ccccc5)cc4)nc(-c4ccc5cc(-c6cccc7oc8ccc(-c9ccc%10ccccc%10c9)cc8c67)ccc5c4)n3)cc2)cc1 ; c1ccc(-c2ccc(-c3nc(-c4ccc(-c5ccccc5)cc4)nc(-c4ccc(-c5cccc6oc7cc(-c8cccc9oc%10ccccc%10c89)ccc7c56)c5ccccc45)n3)cc2)cc1]

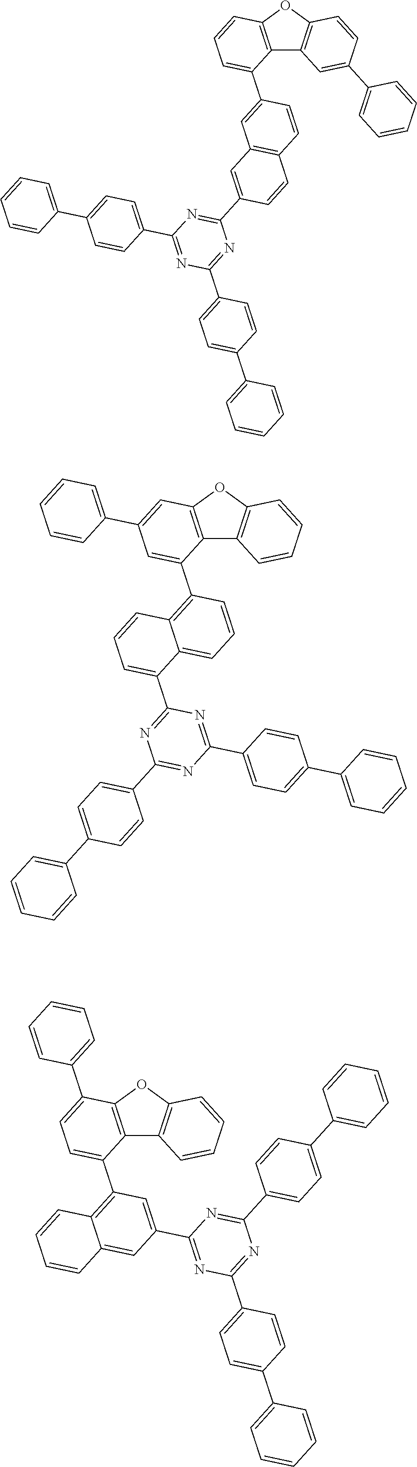 [SMILES: c1ccc(-c2ccc(-c3nc(-c4ccc(-c5ccccc5)cc4)nc(-c4ccc5ccc(-c6cccc7oc8ccc(-c9ccccc9)cc8c67)cc5c4)n3)cc2)cc1 ; c1ccc(-c2ccc(-c3nc(-c4ccc(-c5ccccc5)cc4)nc(-c4cccc5c(-c6cc(-c7ccccc7)cc7oc8ccccc8c67)cccc45)n3)cc2)cc1 ; c1ccc(-c2ccc(-c3nc(-c4ccc(-c5ccccc5)cc4)nc(-c4cc(-c5ccc(-c6ccccc6)c6oc7ccccc7c56)c5ccccc5c4)n3)cc2)cc1]

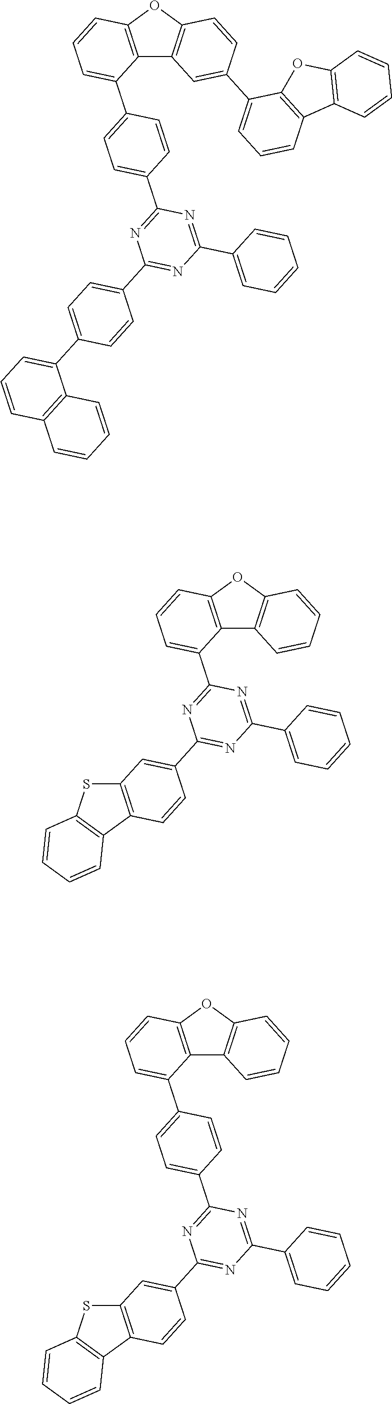

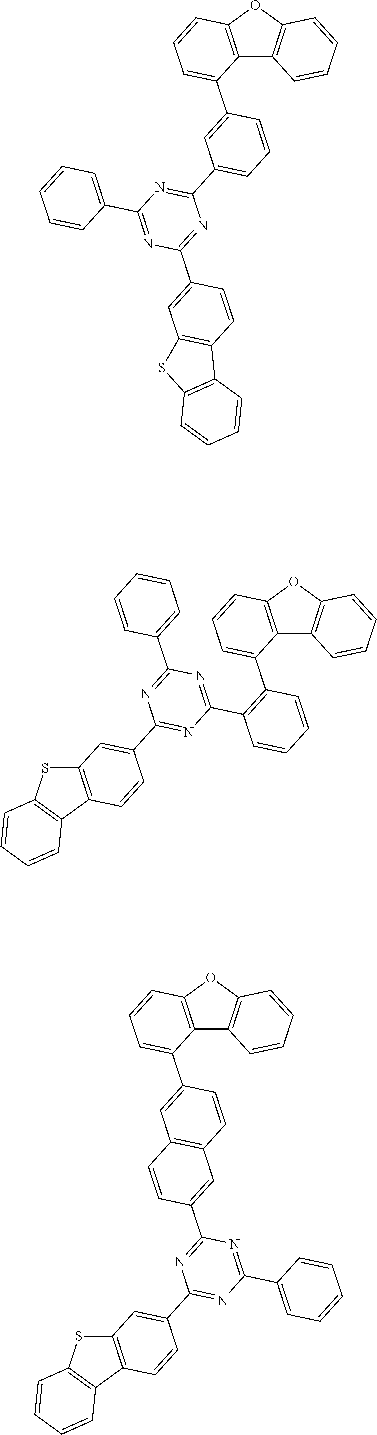 [SMILES: c1ccc(-c2nc(-c3cccc(-c4cccc5oc6ccccc6c45)c3)nc(-c3ccc4c(c3)sc3ccccc34)n2)cc1 ; c1ccc(-c2nc(-c3ccc4c(c3)sc3ccccc34)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4c(c3)sc3ccccc34)n2)cc1]

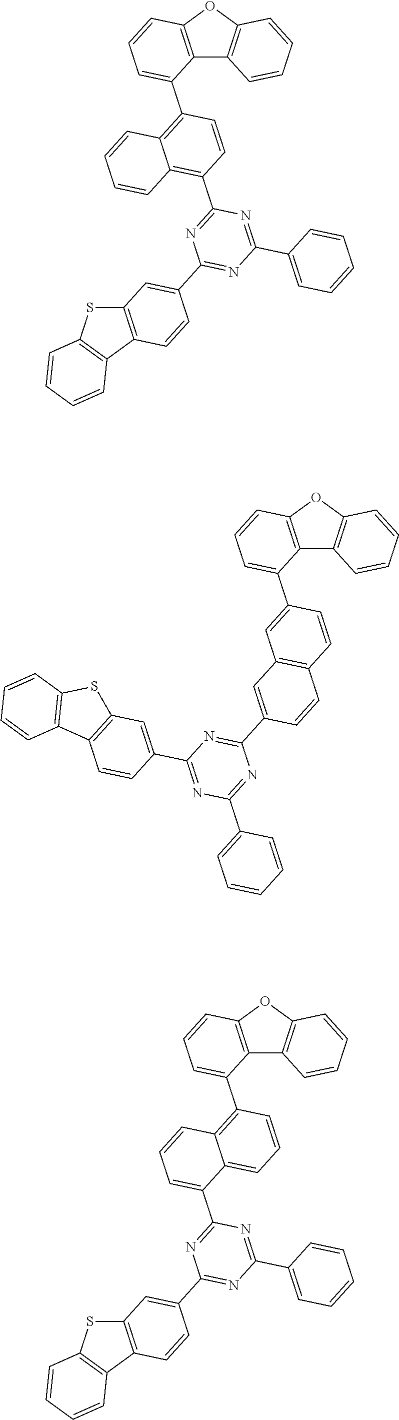 [SMILES: c1ccc(-c2nc(-c3ccc4c(c3)sc3ccccc34)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4c(c3)sc3ccccc34)n2)cc1 ; c1ccc(-c2nc(-c3ccc4c(c3)sc3ccccc34)nc(-c3cccc4c(-c5cccc6oc7ccccc7c56)cccc34)n2)cc1]

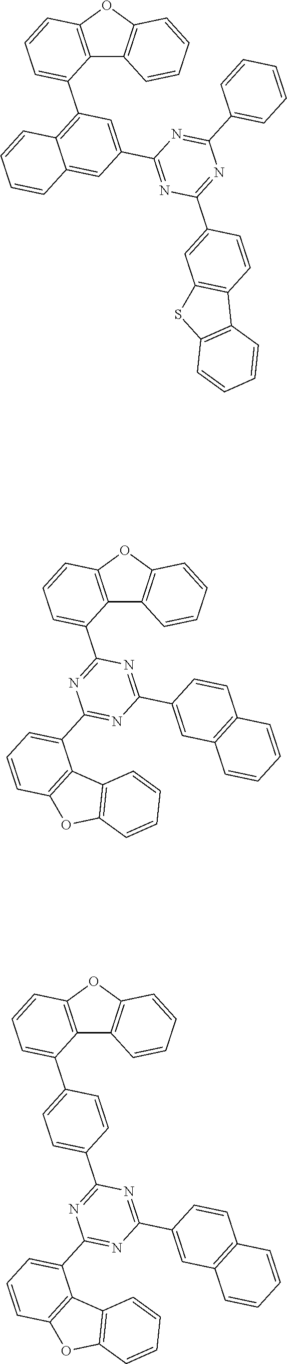 [SMILES: c1ccc(-c2nc(-c3cc(-c4cccc5oc6ccccc6c45)c4ccccc4c3)nc(-c3ccc4c(c3)sc3ccccc34)n2)cc1 ; c1ccc2cc(-c3nc(-c4cccc5oc6ccccc6c45)nc(-c4cccc5oc6ccccc6c45)n3)ccc2c1 ; c1ccc2cc(-c3nc(-c4ccc(-c5cccc6oc7ccccc7c56)cc4)nc(-c4cccc5oc6ccccc6c45)n3)ccc2c1]

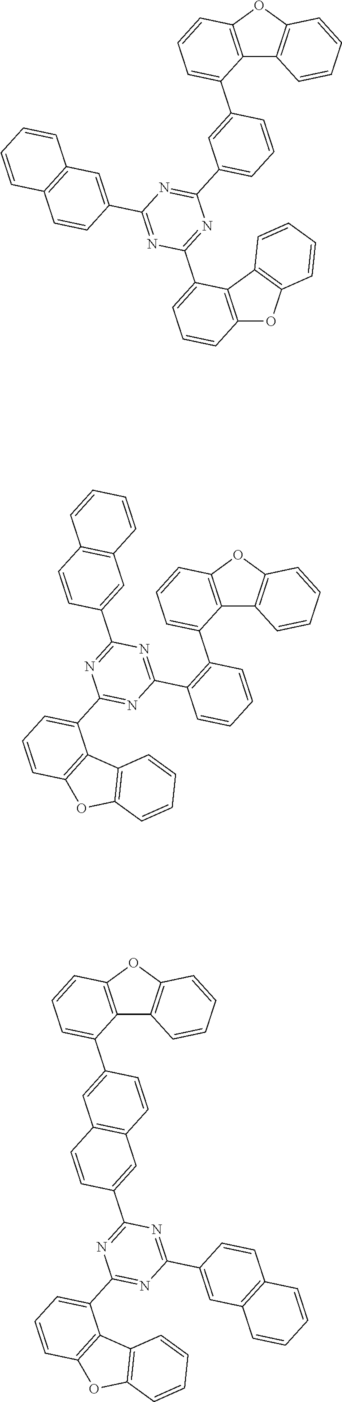 [SMILES: c1cc(-c2nc(-c3ccc4ccccc4c3)nc(-c3cccc4oc5ccccc5c34)n2)cc(-c2cccc3oc4ccccc4c23)c1 ; c1ccc(-c2cccc3oc4ccccc4c23)c(-c2nc(-c3ccc4ccccc4c3)nc(-c3cccc4oc5ccccc5c34)n2)c1 ; c1ccc2cc(-c3nc(-c4ccc5cc(-c6cccc7oc8ccccc8c67)ccc5c4)nc(-c4cccc5oc6ccccc6c45)n3)ccc2c1]

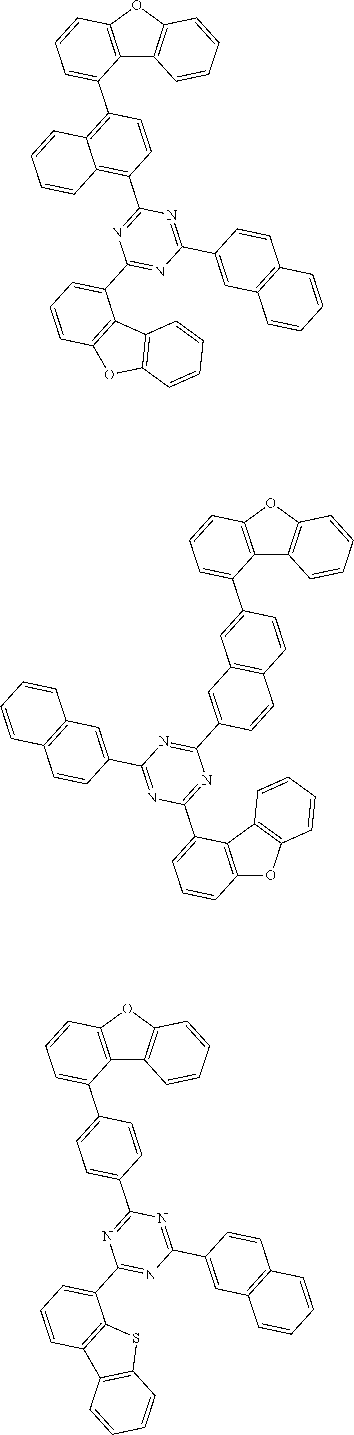 [SMILES: c1ccc2cc(-c3nc(-c4ccc(-c5cccc6oc7ccccc7c56)c5ccccc45)nc(-c4cccc5oc6ccccc6c45)n3)ccc2c1 ; c1ccc2cc(-c3nc(-c4ccc5ccc(-c6cccc7oc8ccccc8c67)cc5c4)nc(-c4cccc5oc6ccccc6c45)n3)ccc2c1 ; c1ccc2cc(-c3nc(-c4ccc(-c5cccc6oc7ccccc7c56)cc4)nc(-c4cccc5c4sc4ccccc45)n3)ccc2c1]

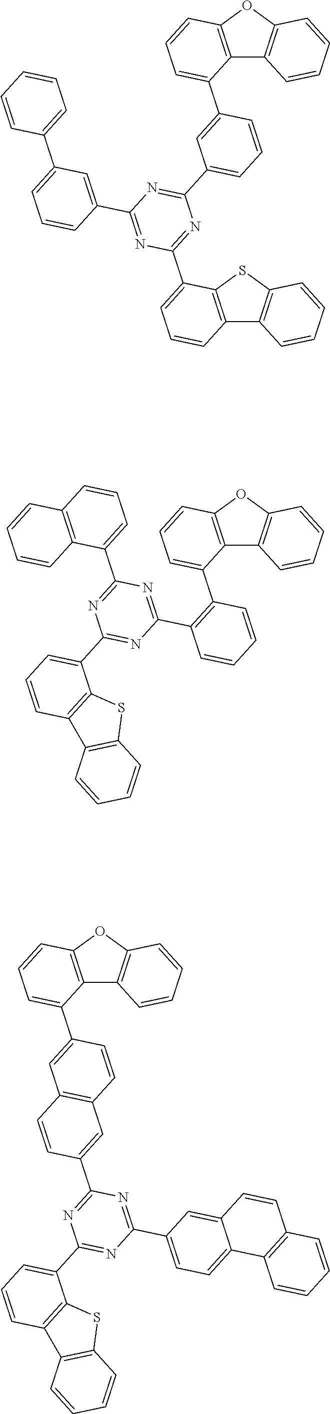 [SMILES: c1ccc(-c2cccc(-c3nc(-c4cccc(-c5cccc6oc7ccccc7c56)c4)nc(-c4cccc5c4sc4ccccc45)n3)c2)cc1 ; c1ccc(-c2cccc3oc4ccccc4c23)c(-c2nc(-c3cccc4ccccc34)nc(-c3cccc4c3sc3ccccc34)n2)c1 ; c1ccc2c(c1)ccc1cc(-c3nc(-c4ccc5cc(-c6cccc7oc8ccccc8c67)ccc5c4)nc(-c4cccc5c4sc4ccccc45)n3)ccc12]

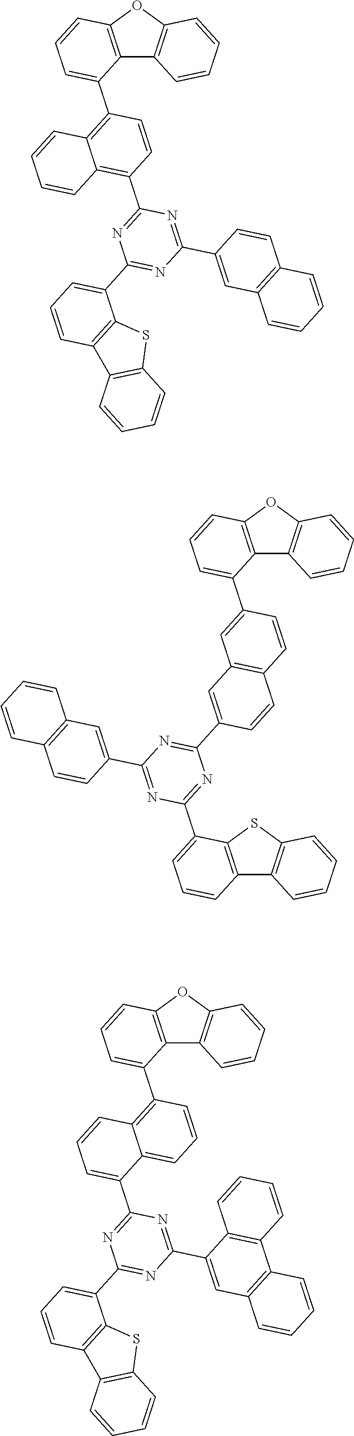 [SMILES: c1ccc2cc(-c3nc(-c4ccc(-c5cccc6oc7ccccc7c56)c5ccccc45)nc(-c4cccc5c4sc4ccccc45)n3)ccc2c1 ; c1ccc2cc(-c3nc(-c4ccc5ccc(-c6cccc7oc8ccccc8c67)cc5c4)nc(-c4cccc5c4sc4ccccc45)n3)ccc2c1 ; c1ccc2c(c1)cc(-c1nc(-c3cccc4c(-c5cccc6oc7ccccc7c56)cccc34)nc(-c3cccc4c3sc3ccccc34)n1)c1ccccc12]

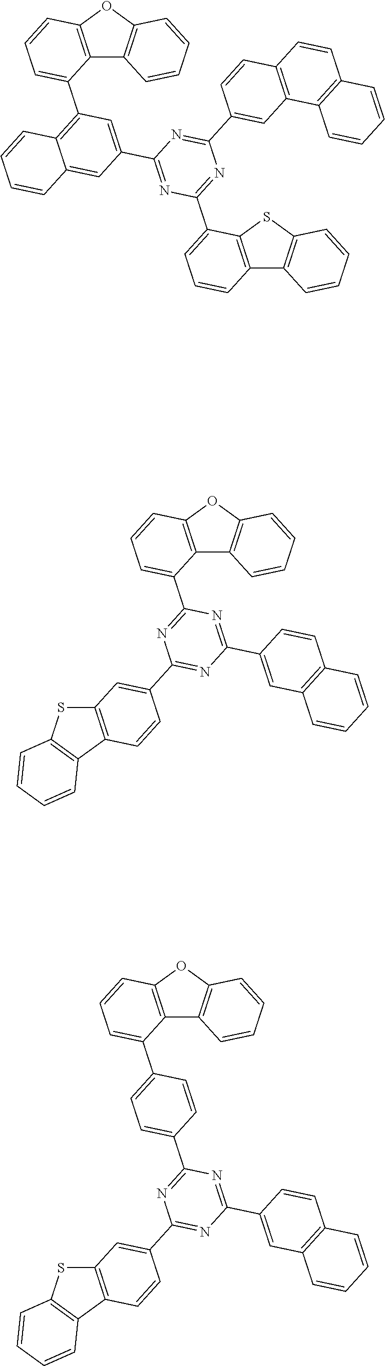 [SMILES: c1ccc2c(-c3cccc4oc5ccccc5c34)cc(-c3nc(-c4ccc5ccc6ccccc6c5c4)nc(-c4cccc5c4sc4ccccc45)n3)cc2c1 ; c1ccc2cc(-c3nc(-c4ccc5c(c4)sc4ccccc45)nc(-c4cccc5oc6ccccc6c45)n3)ccc2c1 ; c1ccc2cc(-c3nc(-c4ccc(-c5cccc6oc7ccccc7c56)cc4)nc(-c4ccc5c(c4)sc4ccccc45)n3)ccc2c1]

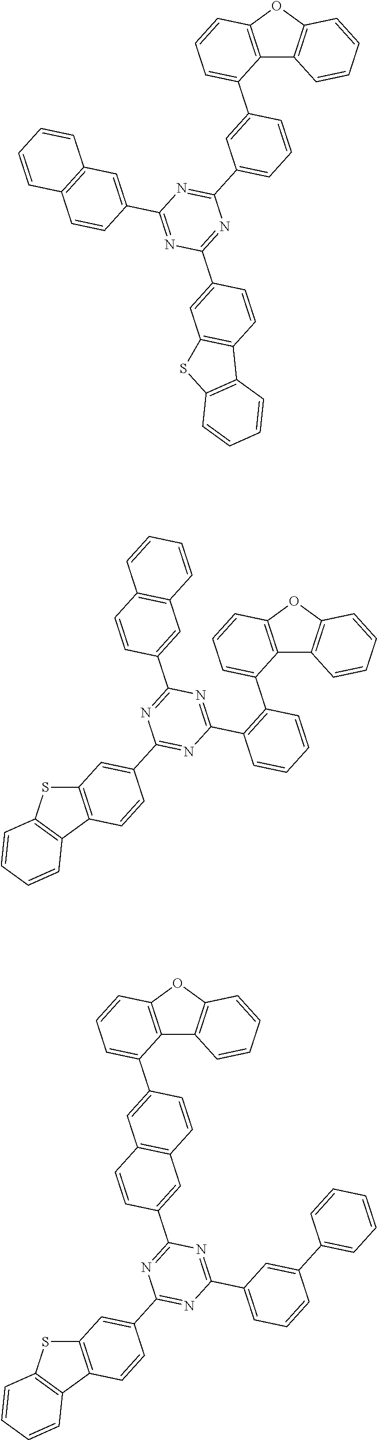 [SMILES: c1cc(-c2nc(-c3ccc4ccccc4c3)nc(-c3ccc4c(c3)sc3ccccc34)n2)cc(-c2cccc3oc4ccccc4c23)c1 ; c1ccc(-c2cccc3oc4ccccc4c23)c(-c2nc(-c3ccc4ccccc4c3)nc(-c3ccc4c(c3)sc3ccccc34)n2)c1 ; c1ccc(-c2cccc(-c3nc(-c4ccc5cc(-c6cccc7oc8ccccc8c67)ccc5c4)nc(-c4ccc5c(c4)sc4ccccc45)n3)c2)cc1]

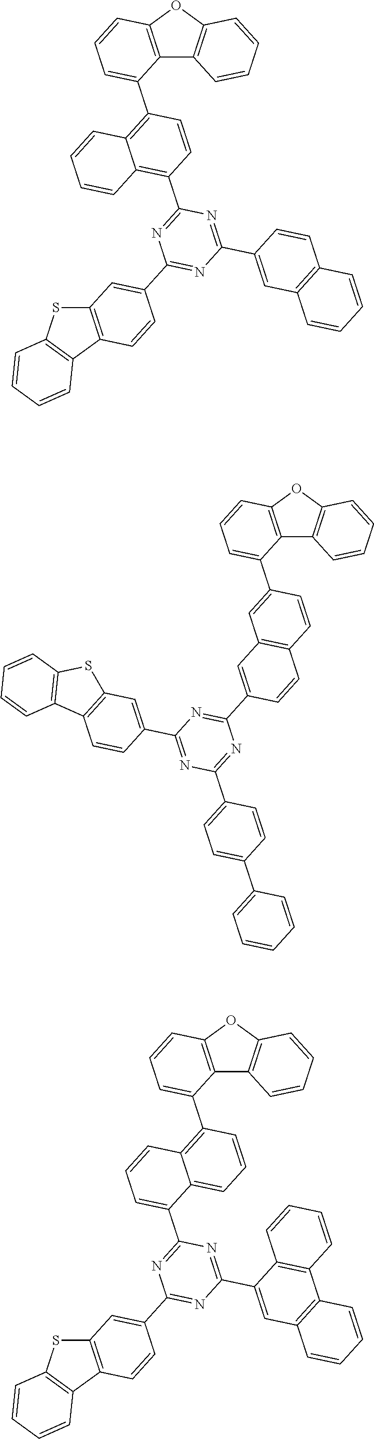 [SMILES: c1ccc2cc(-c3nc(-c4ccc5c(c4)sc4ccccc45)nc(-c4ccc(-c5cccc6oc7ccccc7c56)c5ccccc45)n3)ccc2c1 ; c1ccc(-c2ccc(-c3nc(-c4ccc5ccc(-c6cccc7oc8ccccc8c67)cc5c4)nc(-c4ccc5c(c4)sc4ccccc45)n3)cc2)cc1 ; c1ccc2c(c1)cc(-c1nc(-c3ccc4c(c3)sc3ccccc34)nc(-c3cccc4c(-c5cccc6oc7ccccc7c56)cccc34)n1)c1ccccc12]

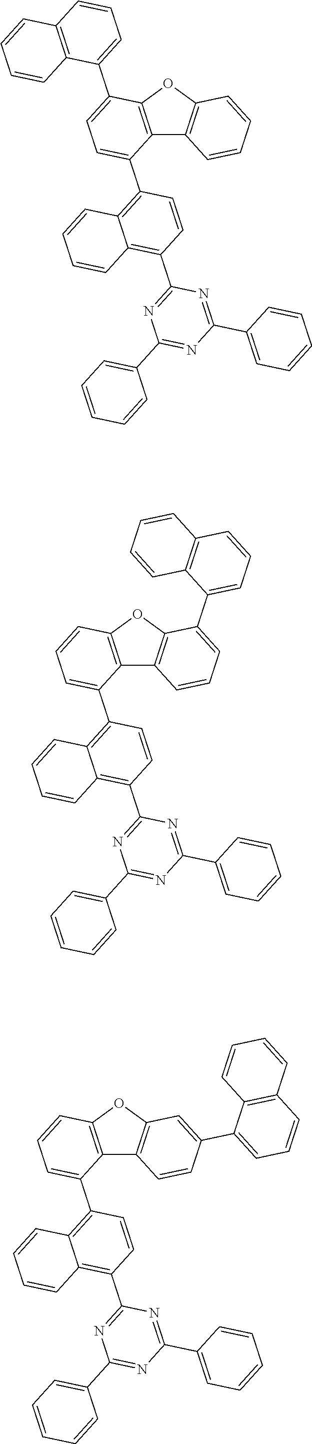 [SMILES: c1ccc(-c2nc(-c3ccccc3)nc(-c3ccc(-c4ccc(-c5cccc6ccccc56)c5oc6ccccc6c45)c4ccccc34)n2)cc1 ; c1ccc(-c2nc(-c3ccccc3)nc(-c3ccc(-c4cccc5oc6c(-c7cccc8ccccc78)cccc6c45)c4ccccc34)n2)cc1 ; c1ccc(-c2nc(-c3ccccc3)nc(-c3ccc(-c4cccc5oc6cc(-c7cccc8ccccc78)ccc6c45)c4ccccc34)n2)cc1]

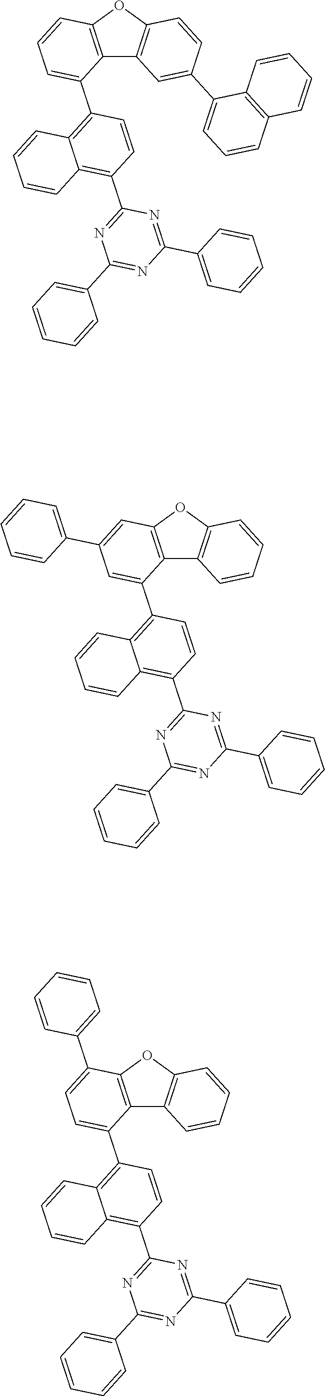 [SMILES: c1ccc(-c2nc(-c3ccccc3)nc(-c3ccc(-c4cccc5oc6ccc(-c7cccc8ccccc78)cc6c45)c4ccccc34)n2)cc1 ; c1ccc(-c2cc(-c3ccc(-c4nc(-c5ccccc5)nc(-c5ccccc5)n4)c4ccccc34)c3c(c2)oc2ccccc23)cc1 ; c1ccc(-c2nc(-c3ccccc3)nc(-c3ccc(-c4ccc(-c5ccccc5)c5oc6ccccc6c45)c4ccccc34)n2)cc1]

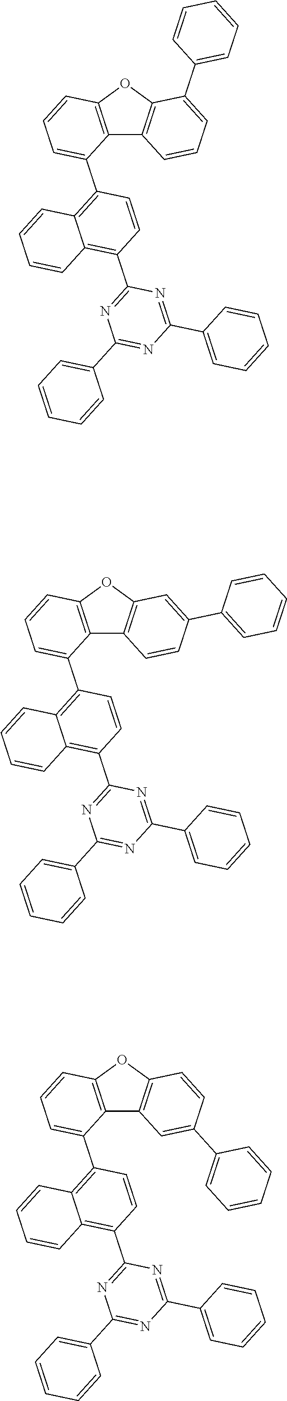 [SMILES: c1ccc(-c2nc(-c3ccccc3)nc(-c3ccc(-c4cccc5oc6c(-c7ccccc7)cccc6c45)c4ccccc34)n2)cc1 ; c1ccc(-c2ccc3c(c2)oc2cccc(-c4ccc(-c5nc(-c6ccccc6)nc(-c6ccccc6)n5)c5ccccc45)c23)cc1 ; c1ccc(-c2ccc3oc4cccc(-c5ccc(-c6nc(-c7ccccc7)nc(-c7ccccc7)n6)c6ccccc56)c4c3c2)cc1]

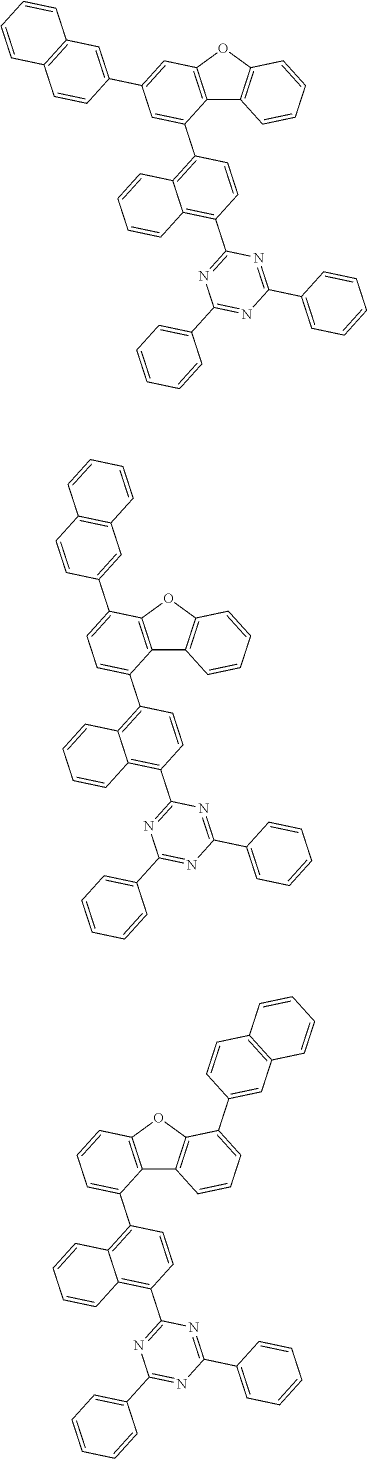 [SMILES: c1ccc(-c2nc(-c3ccccc3)nc(-c3ccc(-c4cc(-c5ccc6ccccc6c5)cc5oc6ccccc6c45)c4ccccc34)n2)cc1 ; c1ccc(-c2nc(-c3ccccc3)nc(-c3ccc(-c4ccc(-c5ccc6ccccc6c5)c5oc6ccccc6c45)c4ccccc34)n2)cc1 ; c1ccc(-c2nc(-c3ccccc3)nc(-c3ccc(-c4cccc5oc6c(-c7ccc8ccccc8c7)cccc6c45)c4ccccc34)n2)cc1]

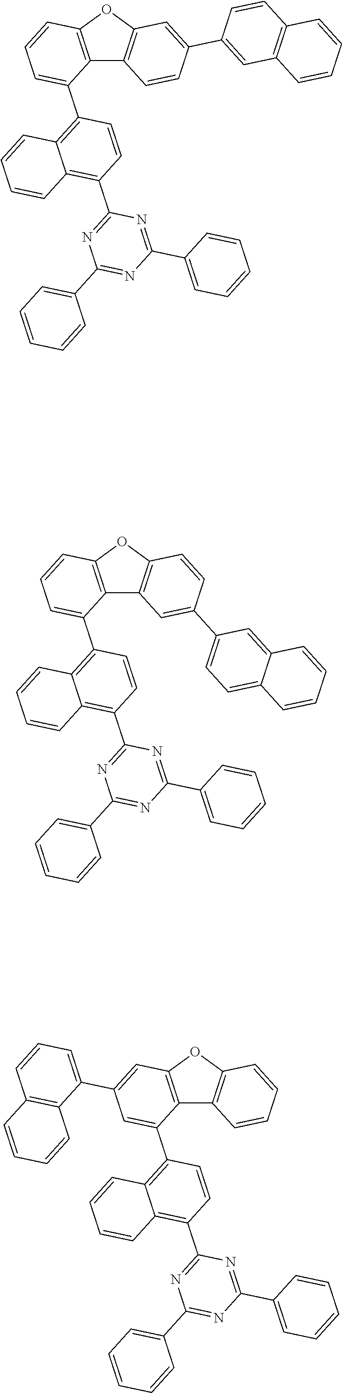 [SMILES: c1ccc(-c2nc(-c3ccccc3)nc(-c3ccc(-c4cccc5oc6cc(-c7ccc8ccccc8c7)ccc6c45)c4ccccc34)n2)cc1 ; c1ccc(-c2nc(-c3ccccc3)nc(-c3ccc(-c4cccc5oc6ccc(-c7ccc8ccccc8c7)cc6c45)c4ccccc34)n2)cc1 ; c1ccc(-c2nc(-c3ccccc3)nc(-c3ccc(-c4cc(-c5cccc6ccccc56)cc5oc6ccccc6c45)c4ccccc34)n2)cc1]

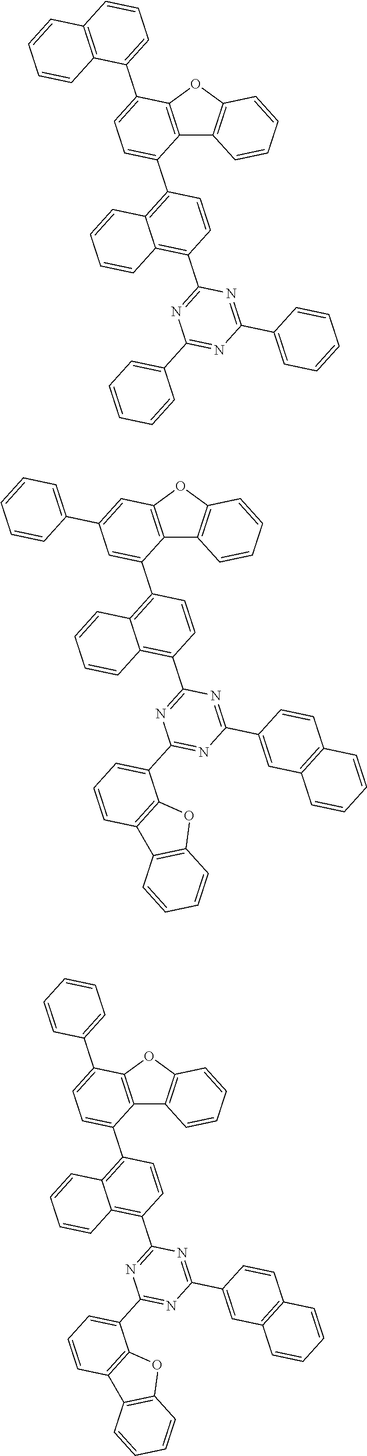 [SMILES: c1ccc(-c2nc(-c3ccccc3)nc(-c3ccc(-c4ccc(-c5cccc6ccccc56)c5oc6ccccc6c45)c4ccccc34)n2)cc1 ; c1ccc(-c2cc(-c3ccc(-c4nc(-c5ccc6ccccc6c5)nc(-c5cccc6c5oc5ccccc56)n4)c4ccccc34)c3c(c2)oc2ccccc23)cc1 ; c1ccc(-c2ccc(-c3ccc(-c4nc(-c5ccc6ccccc6c5)nc(-c5cccc6c5oc5ccccc56)n4)c4ccccc34)c3c2oc2ccccc23)cc1]

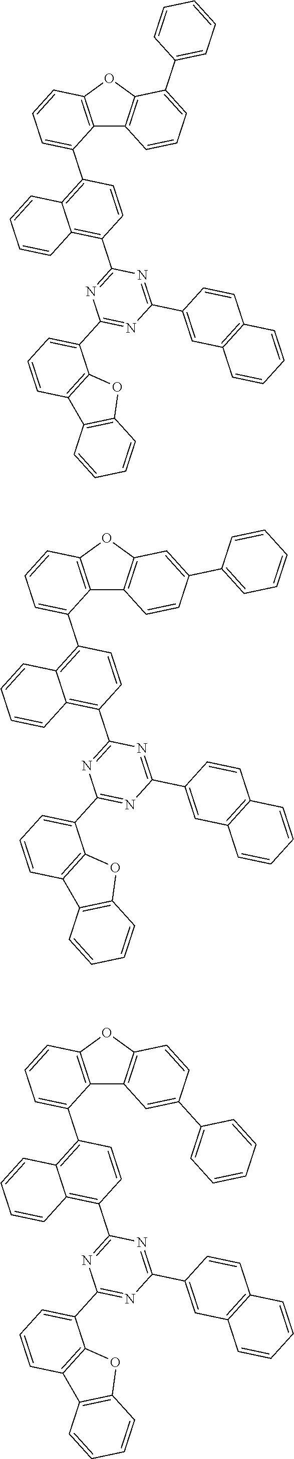 [SMILES: c1ccc(-c2cccc3c2oc2cccc(-c4ccc(-c5nc(-c6ccc7ccccc7c6)nc(-c6cccc7c6oc6ccccc67)n5)c5ccccc45)c23)cc1 ; c1ccc(-c2ccc3c(c2)oc2cccc(-c4ccc(-c5nc(-c6ccc7ccccc7c6)nc(-c6cccc7c6oc6ccccc67)n5)c5ccccc45)c23)cc1 ; c1ccc(-c2ccc3oc4cccc(-c5ccc(-c6nc(-c7ccc8ccccc8c7)nc(-c7cccc8c7oc7ccccc78)n6)c6ccccc56)c4c3c2)cc1]

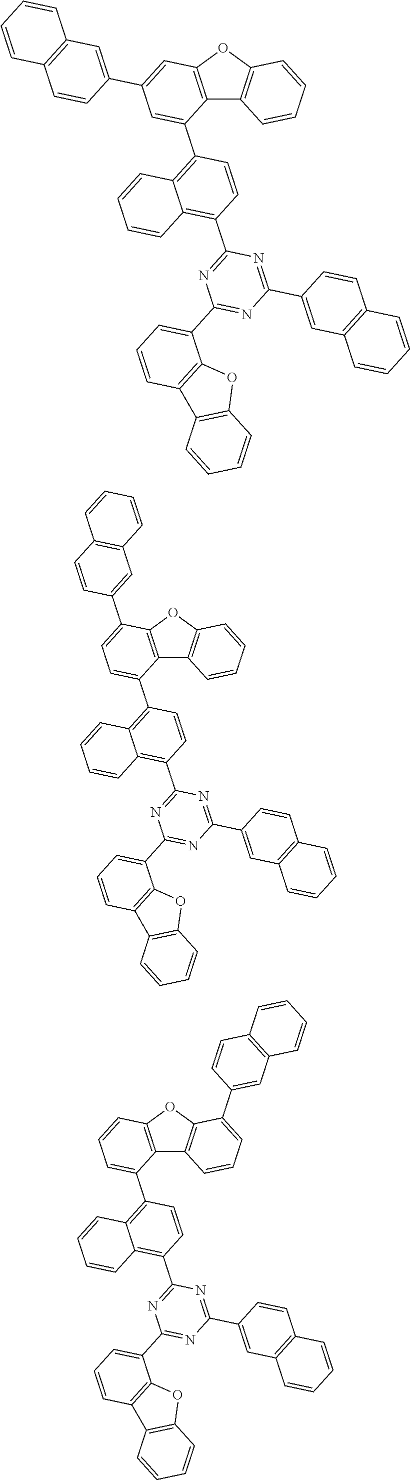 [SMILES: c1ccc2cc(-c3cc(-c4ccc(-c5nc(-c6ccc7ccccc7c6)nc(-c6cccc7c6oc6ccccc67)n5)c5ccccc45)c4c(c3)oc3ccccc34)ccc2c1 ; c1ccc2cc(-c3nc(-c4ccc(-c5ccc(-c6ccc7ccccc7c6)c6oc7ccccc7c56)c5ccccc45)nc(-c4cccc5c4oc4ccccc45)n3)ccc2c1 ; c1ccc2cc(-c3nc(-c4ccc(-c5cccc6oc7c(-c8ccc9ccccc9c8)cccc7c56)c5ccccc45)nc(-c4cccc5c4oc4ccccc45)n3)ccc2c1]

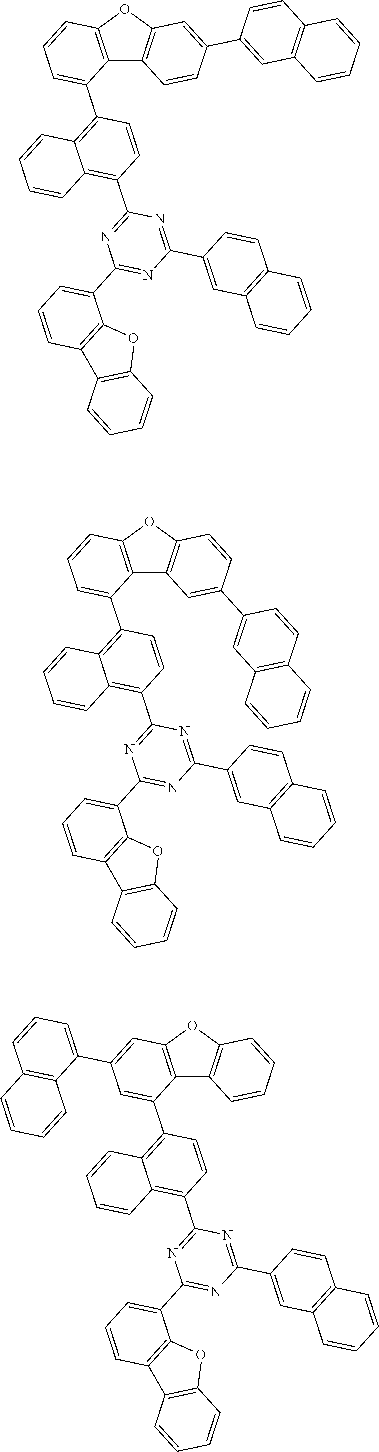 [SMILES: c1ccc2cc(-c3ccc4c(c3)oc3cccc(-c5ccc(-c6nc(-c7ccc8ccccc8c7)nc(-c7cccc8c7oc7ccccc78)n6)c6ccccc56)c34)ccc2c1 ; c1ccc2cc(-c3ccc4oc5cccc(-c6ccc(-c7nc(-c8ccc9ccccc9c8)nc(-c8cccc9c8oc8ccccc89)n7)c7ccccc67)c5c4c3)ccc2c1 ; c1ccc2cc(-c3nc(-c4ccc(-c5cc(-c6cccc7ccccc67)cc6oc7ccccc7c56)c5ccccc45)nc(-c4cccc5c4oc4ccccc45)n3)ccc2c1]

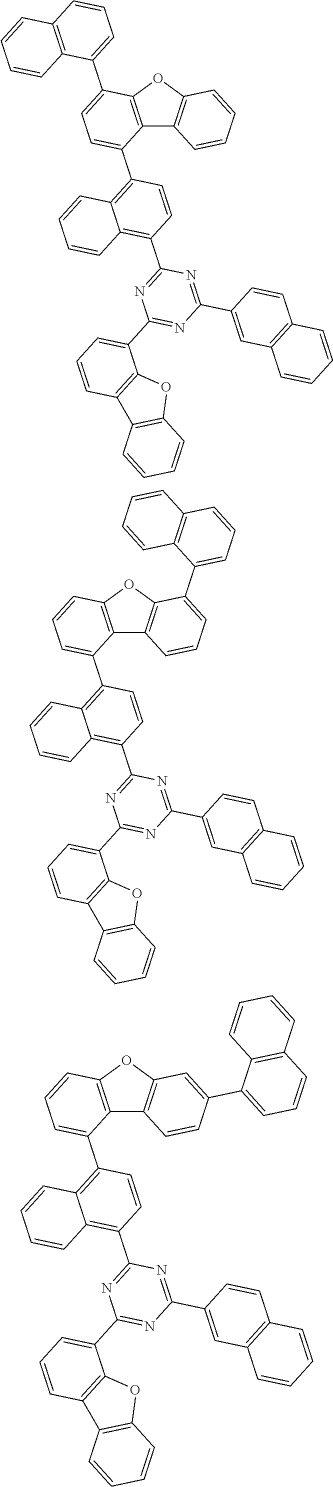

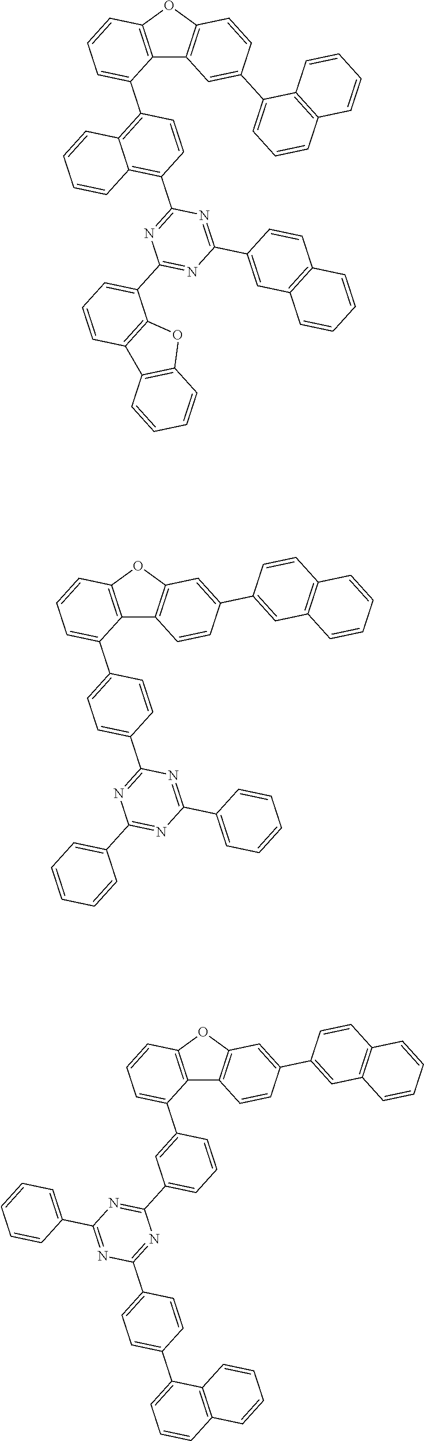 [SMILES: c1ccc2cc(-c3nc(-c4ccc(-c5cccc6oc7ccc(-c8cccc9ccccc89)cc7c56)c5ccccc45)nc(-c4cccc5c4oc4ccccc45)n3)ccc2c1 ; c1ccc(-c2nc(-c3ccccc3)nc(-c3ccc(-c4cccc5oc6cc(-c7ccc8ccccc8c7)ccc6c45)cc3)n2)cc1 ; c1ccc(-c2nc(-c3ccc(-c4cccc5ccccc45)cc3)nc(-c3cccc(-c4cccc5oc6cc(-c7ccc8ccccc8c7)ccc6c45)c3)n2)cc1]

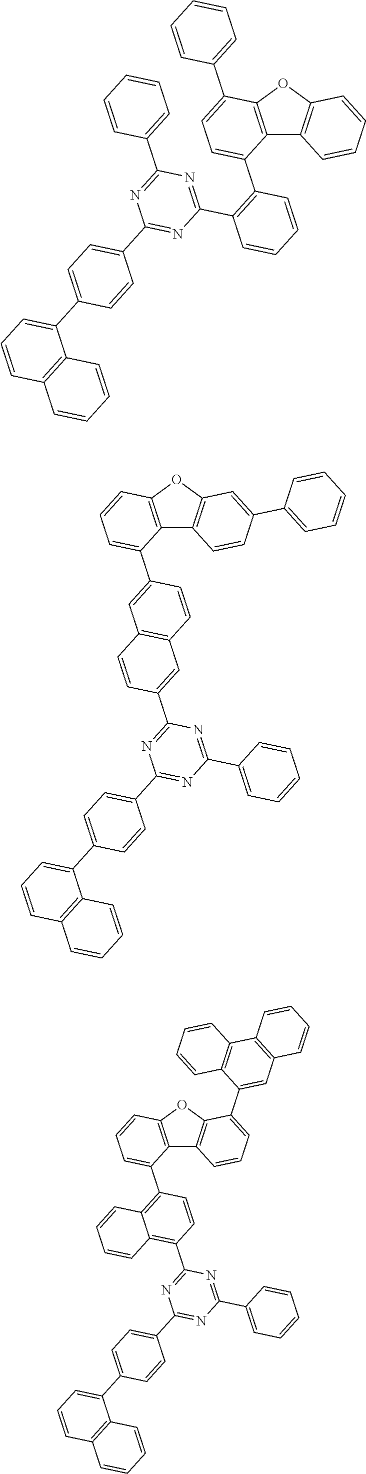 [SMILES: c1ccc(-c2nc(-c3ccc(-c4cccc5ccccc45)cc3)nc(-c3ccccc3-c3ccc(-c4ccccc4)c4oc5ccccc5c34)n2)cc1 ; c1ccc(-c2ccc3c(c2)oc2cccc(-c4ccc5cc(-c6nc(-c7ccccc7)nc(-c7ccc(-c8cccc9ccccc89)cc7)n6)ccc5c4)c23)cc1 ; c1ccc(-c2nc(-c3ccc(-c4cccc5ccccc45)cc3)nc(-c3ccc(-c4cccc5oc6c(-c7cc8ccccc8c8ccccc78)cccc6c45)c4ccccc34)n2)cc1]

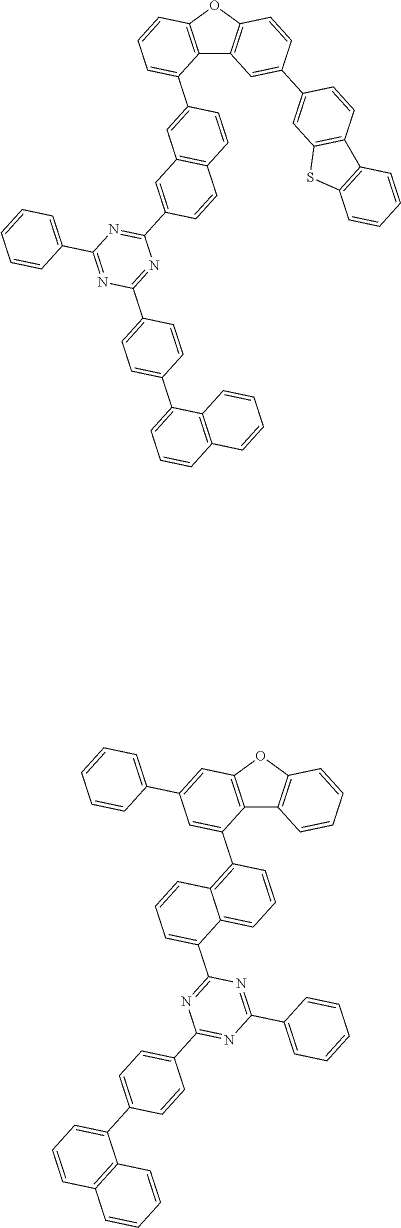 [SMILES: c1ccc(-c2nc(-c3ccc(-c4cccc5ccccc45)cc3)nc(-c3ccc4ccc(-c5cccc6oc7ccc(-c8ccc9c(c8)sc8ccccc89)cc7c56)cc4c3)n2)cc1 ; c1ccc(-c2cc(-c3cccc4c(-c5nc(-c6ccccc6)nc(-c6ccc(-c7cccc8ccccc78)cc6)n5)cccc34)c3c(c2)oc2ccccc23)cc1]

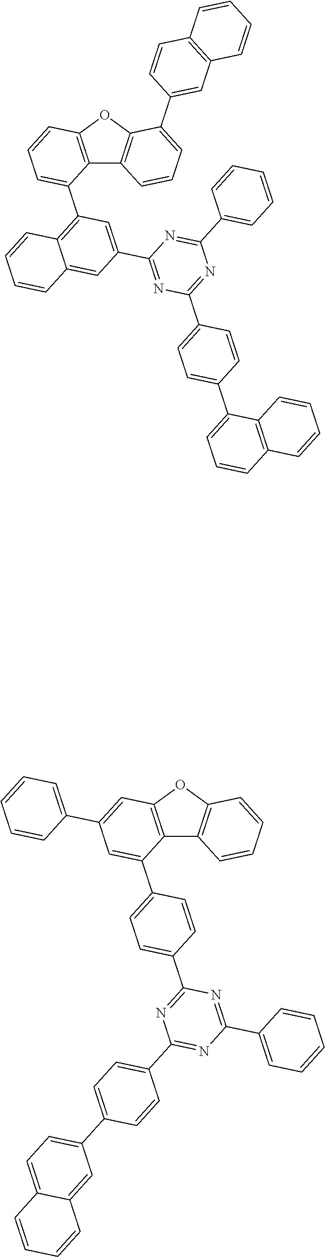 [SMILES: c1ccc(-c2nc(-c3ccc(-c4cccc5ccccc45)cc3)nc(-c3cc(-c4cccc5oc6c(-c7ccc8ccccc8c7)cccc6c45)c4ccccc4c3)n2)cc1 ; c1ccc(-c2cc(-c3ccc(-c4nc(-c5ccccc5)nc(-c5ccc(-c6ccc7ccccc7c6)cc5)n4)cc3)c3c(c2)oc2ccccc23)cc1]

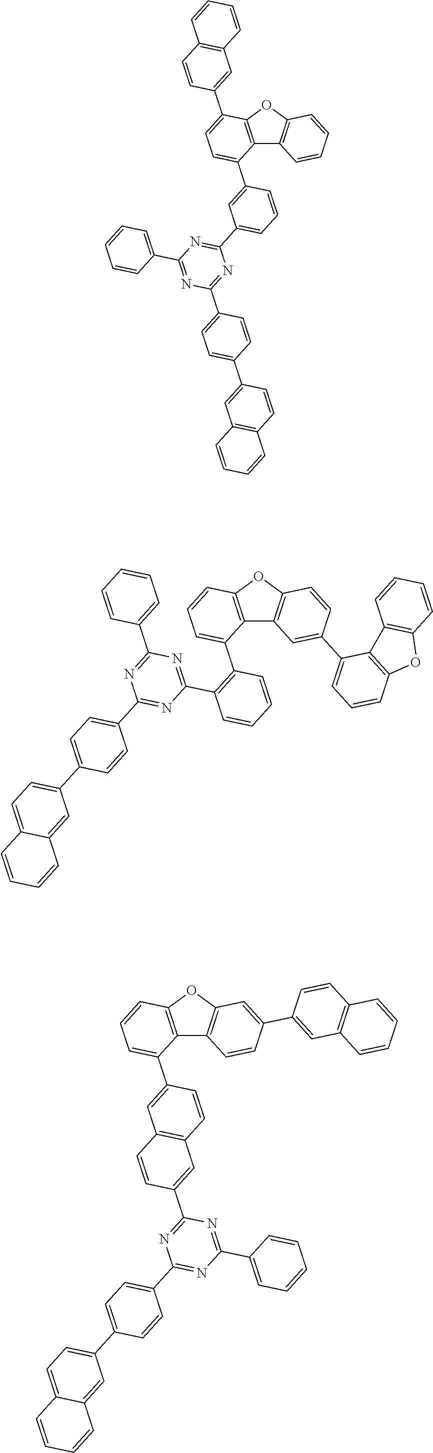 [SMILES: c1ccc(-c2nc(-c3ccc(-c4ccc5ccccc5c4)cc3)nc(-c3cccc(-c4ccc(-c5ccc6ccccc6c5)c5oc6ccccc6c45)c3)n2)cc1 ; c1ccc(-c2nc(-c3ccc(-c4ccc5ccccc5c4)cc3)nc(-c3ccccc3-c3cccc4oc5ccc(-c6cccc7oc8ccccc8c67)cc5c34)n2)cc1 ; c1ccc(-c2nc(-c3ccc(-c4ccc5ccccc5c4)cc3)nc(-c3ccc4cc(-c5cccc6oc7cc(-c8ccc9ccccc9c8)ccc7c56)ccc4c3)n2)cc1]

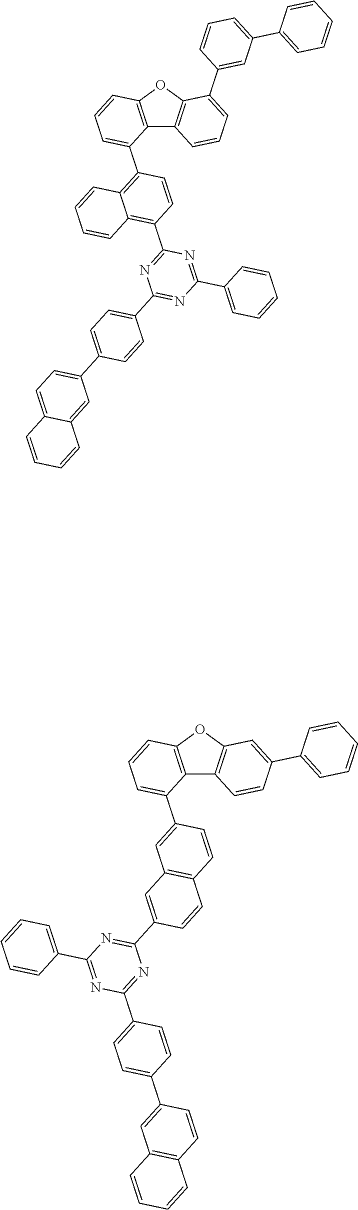 [SMILES: c1ccc(-c2cccc(-c3cccc4c3oc3cccc(-c5ccc(-c6nc(-c7ccccc7)nc(-c7ccc(-c8ccc9ccccc9c8)cc7)n6)c6ccccc56)c34)c2)cc1 ; c1ccc(-c2ccc3c(c2)oc2cccc(-c4ccc5ccc(-c6nc(-c7ccccc7)nc(-c7ccc(-c8ccc9ccccc9c8)cc7)n6)cc5c4)c23)cc1]

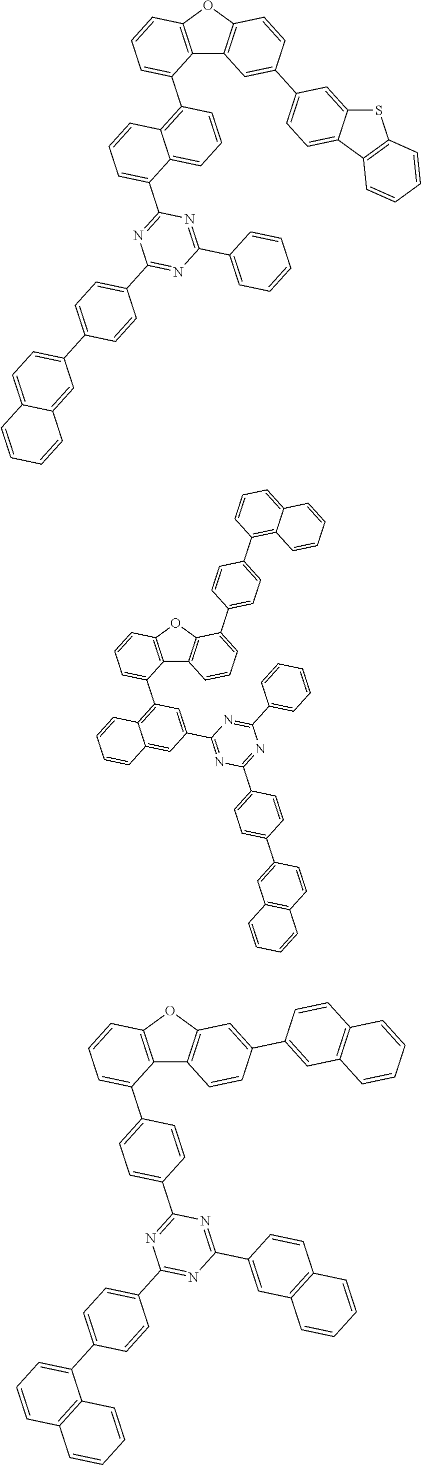 [SMILES: c1ccc(-c2nc(-c3ccc(-c4ccc5ccccc5c4)cc3)nc(-c3cccc4c(-c5cccc6oc7ccc(-c8ccc9c(c8)sc8ccccc89)cc7c56)cccc34)n2)cc1 ; c1ccc(-c2nc(-c3ccc(-c4ccc5ccccc5c4)cc3)nc(-c3cc(-c4cccc5oc6c(-c7ccc(-c8cccc9ccccc89)cc7)cccc6c45)c4ccccc4c3)n2)cc1 ; c1ccc2cc(-c3ccc4c(c3)oc3cccc(-c5ccc(-c6nc(-c7ccc(-c8cccc9ccccc89)cc7)nc(-c7ccc8ccccc8c7)n6)cc5)c34)ccc2c1]

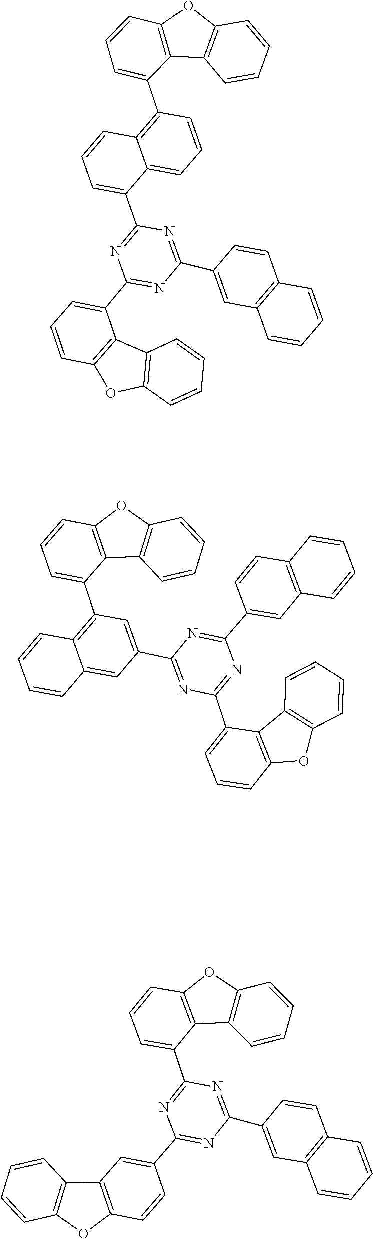 [SMILES: c1ccc2cc(-c3nc(-c4cccc5c(-c6cccc7oc8ccccc8c67)cccc45)nc(-c4cccc5oc6ccccc6c45)n3)ccc2c1 ; c1ccc2cc(-c3nc(-c4cc(-c5cccc6oc7ccccc7c56)c5ccccc5c4)nc(-c4cccc5oc6ccccc6c45)n3)ccc2c1 ; c1ccc2cc(-c3nc(-c4ccc5oc6ccccc6c5c4)nc(-c4cccc5oc6ccccc6c45)n3)ccc2c1]

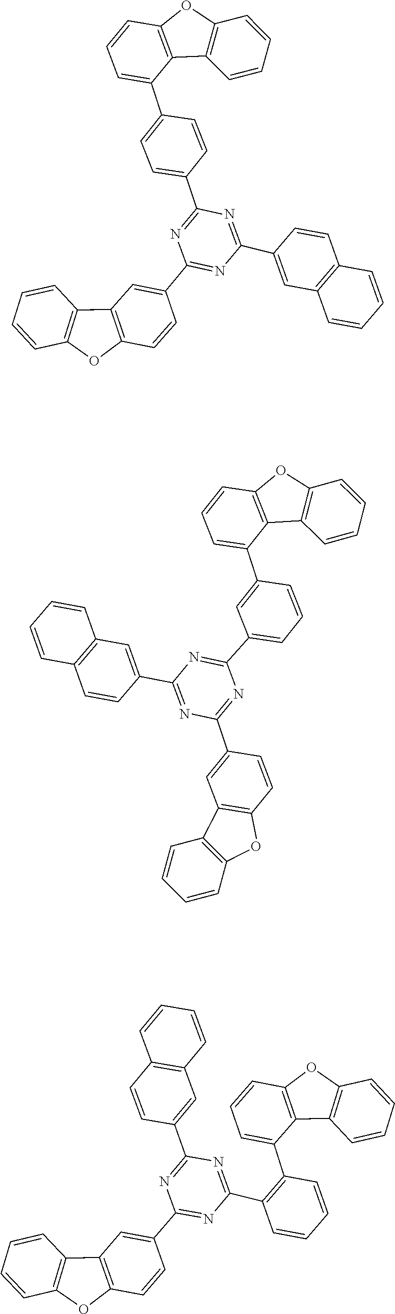 [SMILES: c1ccc2cc(-c3nc(-c4ccc(-c5cccc6oc7ccccc7c56)cc4)nc(-c4ccc5oc6ccccc6c5c4)n3)ccc2c1 ; c1cc(-c2nc(-c3ccc4ccccc4c3)nc(-c3ccc4oc5ccccc5c4c3)n2)cc(-c2cccc3oc4ccccc4c23)c1 ; c1ccc(-c2cccc3oc4ccccc4c23)c(-c2nc(-c3ccc4ccccc4c3)nc(-c3ccc4oc5ccccc5c4c3)n2)c1]

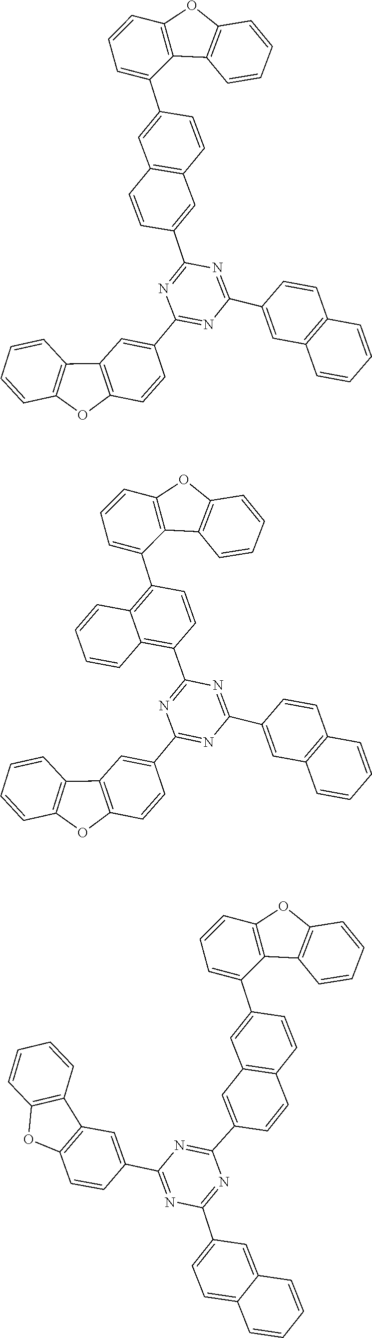 [SMILES: c1ccc2cc(-c3nc(-c4ccc5cc(-c6cccc7oc8ccccc8c67)ccc5c4)nc(-c4ccc5oc6ccccc6c5c4)n3)ccc2c1 ; c1ccc2cc(-c3nc(-c4ccc5oc6ccccc6c5c4)nc(-c4ccc(-c5cccc6oc7ccccc7c56)c5ccccc45)n3)ccc2c1 ; c1ccc2cc(-c3nc(-c4ccc5ccc(-c6cccc7oc8ccccc8c67)cc5c4)nc(-c4ccc5oc6ccccc6c5c4)n3)ccc2c1]

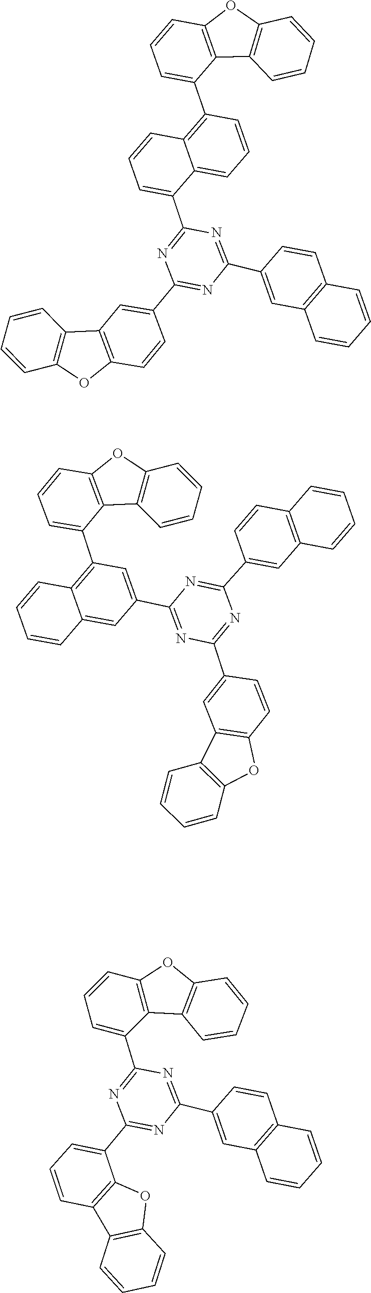 [SMILES: c1ccc2cc(-c3nc(-c4ccc5oc6ccccc6c5c4)nc(-c4cccc5c(-c6cccc7oc8ccccc8c67)cccc45)n3)ccc2c1 ; c1ccc2cc(-c3nc(-c4cc(-c5cccc6oc7ccccc7c56)c5ccccc5c4)nc(-c4ccc5oc6ccccc6c5c4)n3)ccc2c1 ; c1ccc2cc(-c3nc(-c4cccc5c4oc4ccccc45)nc(-c4cccc5oc6ccccc6c45)n3)ccc2c1]

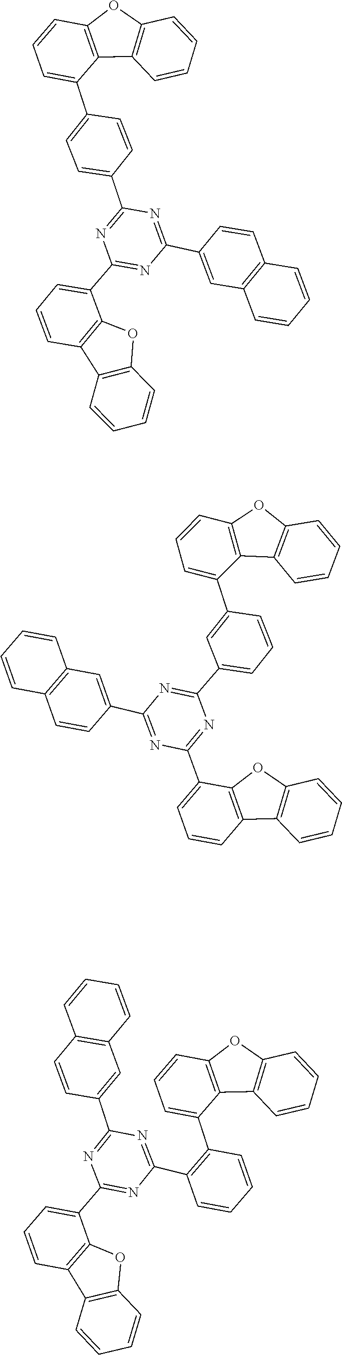 [SMILES: c1ccc2cc(-c3nc(-c4ccc(-c5cccc6oc7ccccc7c56)cc4)nc(-c4cccc5c4oc4ccccc45)n3)ccc2c1 ; c1cc(-c2nc(-c3ccc4ccccc4c3)nc(-c3cccc4c3oc3ccccc34)n2)cc(-c2cccc3oc4ccccc4c23)c1 ; c1ccc(-c2cccc3oc4ccccc4c23)c(-c2nc(-c3ccc4ccccc4c3)nc(-c3cccc4c3oc3ccccc34)n2)c1]

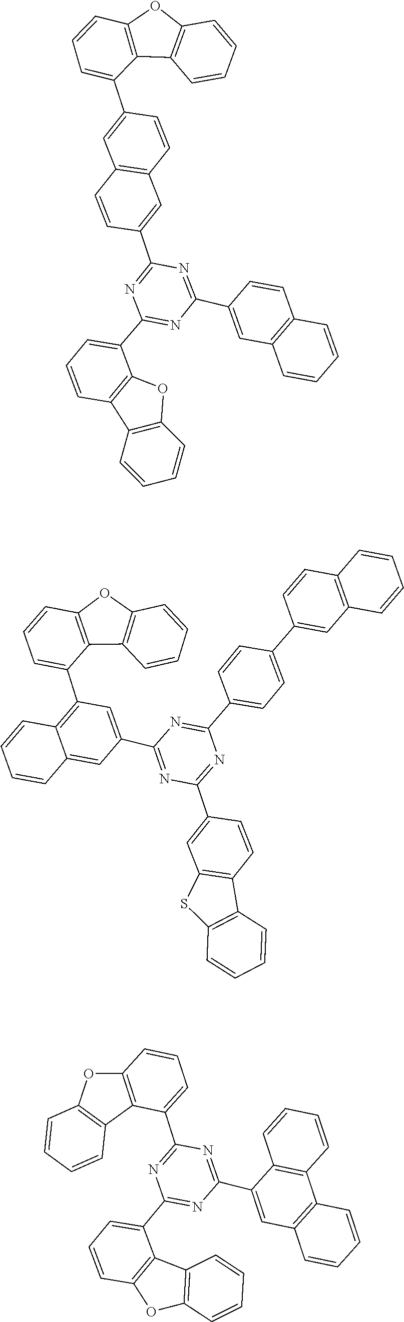 [SMILES: c1ccc2cc(-c3nc(-c4ccc5cc(-c6cccc7oc8ccccc8c67)ccc5c4)nc(-c4cccc5c4oc4ccccc45)n3)ccc2c1 ; c1ccc2cc(-c3ccc(-c4nc(-c5cc(-c6cccc7oc8ccccc8c67)c6ccccc6c5)nc(-c5ccc6c(c5)sc5ccccc56)n4)cc3)ccc2c1 ; c1ccc2c(c1)cc(-c1nc(-c3cccc4oc5ccccc5c34)nc(-c3cccc4oc5ccccc5c34)n1)c1ccccc12]

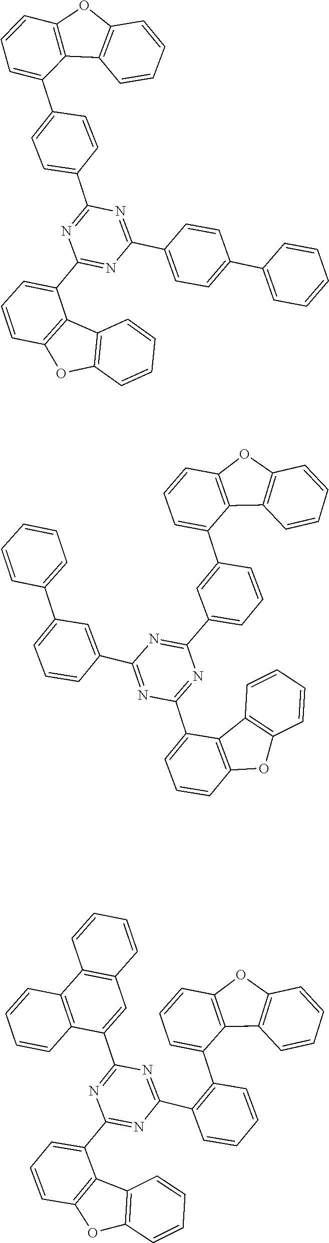 [SMILES: c1ccc(-c2ccc(-c3nc(-c4ccc(-c5cccc6oc7ccccc7c56)cc4)nc(-c4cccc5oc6ccccc6c45)n3)cc2)cc1 ; c1ccc(-c2cccc(-c3nc(-c4cccc(-c5cccc6oc7ccccc7c56)c4)nc(-c4cccc5oc6ccccc6c45)n3)c2)cc1 ; c1ccc(-c2cccc3oc4ccccc4c23)c(-c2nc(-c3cc4ccccc4c4ccccc34)nc(-c3cccc4oc5ccccc5c34)n2)c1]

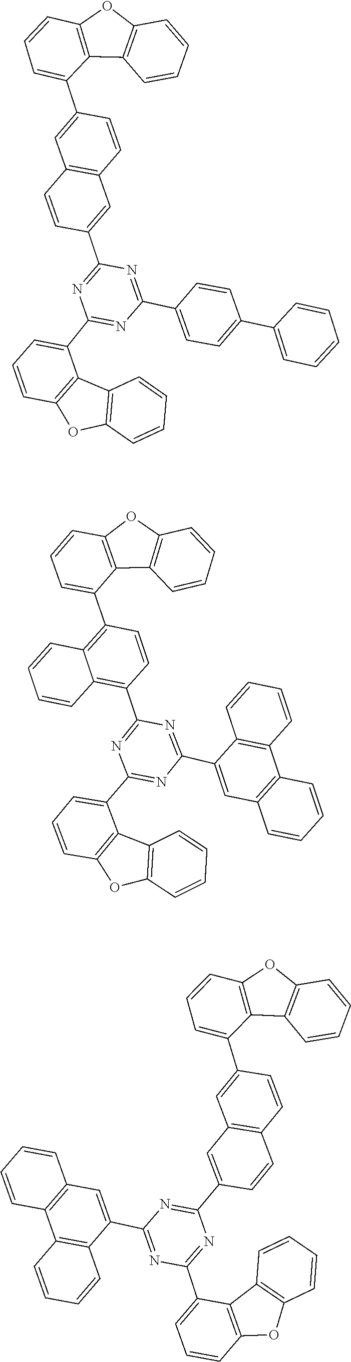 [SMILES: c1ccc(-c2ccc(-c3nc(-c4ccc5cc(-c6cccc7oc8ccccc8c67)ccc5c4)nc(-c4cccc5oc6ccccc6c45)n3)cc2)cc1 ; c1ccc2c(c1)cc(-c1nc(-c3ccc(-c4cccc5oc6ccccc6c45)c4ccccc34)nc(-c3cccc4oc5ccccc5c34)n1)c1ccccc12 ; c1ccc2c(c1)cc(-c1nc(-c3ccc4ccc(-c5cccc6oc7ccccc7c56)cc4c3)nc(-c3cccc4oc5ccccc5c34)n1)c1ccccc12]

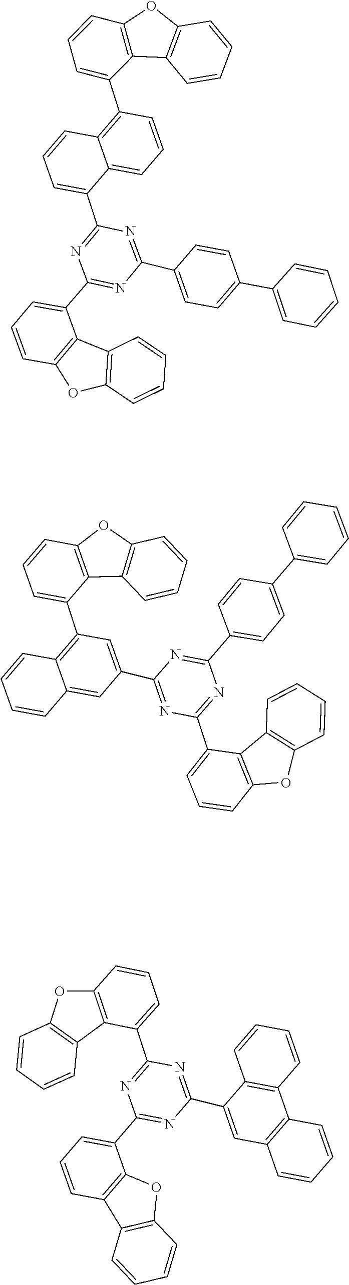 [SMILES: c1ccc(-c2ccc(-c3nc(-c4cccc5c(-c6cccc7oc8ccccc8c67)cccc45)nc(-c4cccc5oc6ccccc6c45)n3)cc2)cc1 ; c1ccc(-c2ccc(-c3nc(-c4cc(-c5cccc6oc7ccccc7c56)c5ccccc5c4)nc(-c4cccc5oc6ccccc6c45)n3)cc2)cc1 ; c1ccc2c(c1)cc(-c1nc(-c3cccc4c3oc3ccccc34)nc(-c3cccc4oc5ccccc5c34)n1)c1ccccc12]

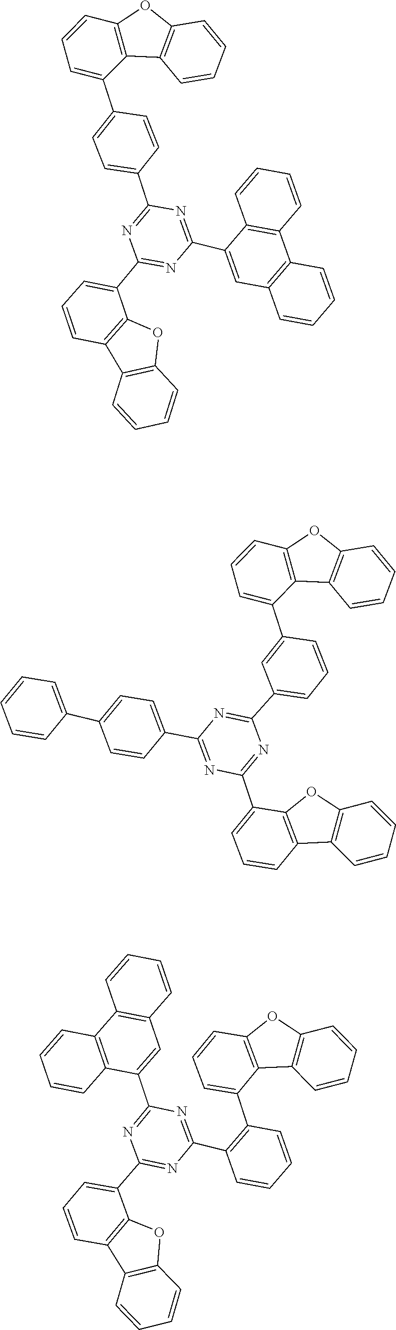 [SMILES: c1ccc2c(c1)cc(-c1nc(-c3ccc(-c4cccc5oc6ccccc6c45)cc3)nc(-c3cccc4c3oc3ccccc34)n1)c1ccccc12 ; c1ccc(-c2ccc(-c3nc(-c4cccc(-c5cccc6oc7ccccc7c56)c4)nc(-c4cccc5c4oc4ccccc45)n3)cc2)cc1 ; c1ccc(-c2cccc3oc4ccccc4c23)c(-c2nc(-c3cc4ccccc4c4ccccc34)nc(-c3cccc4c3oc3ccccc34)n2)c1]

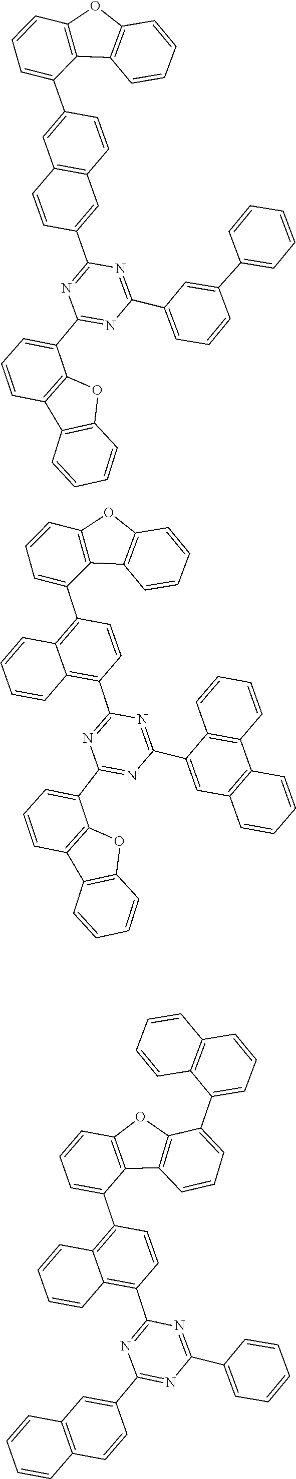

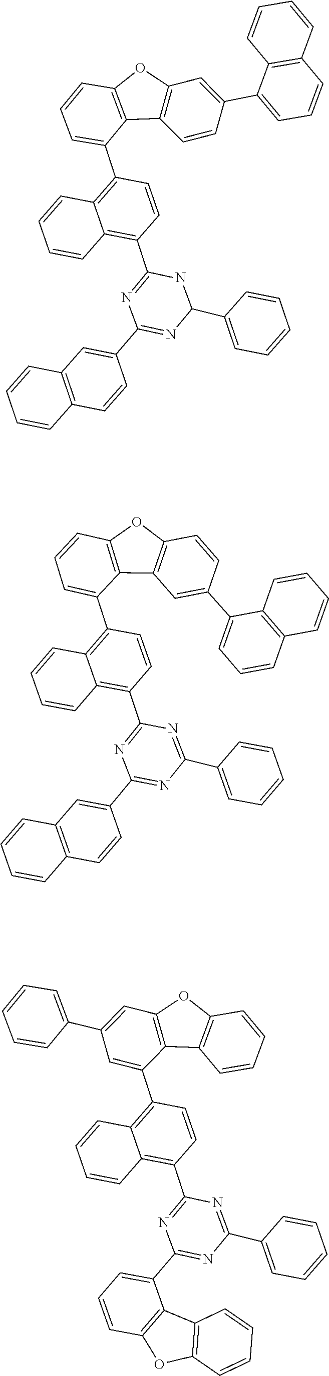

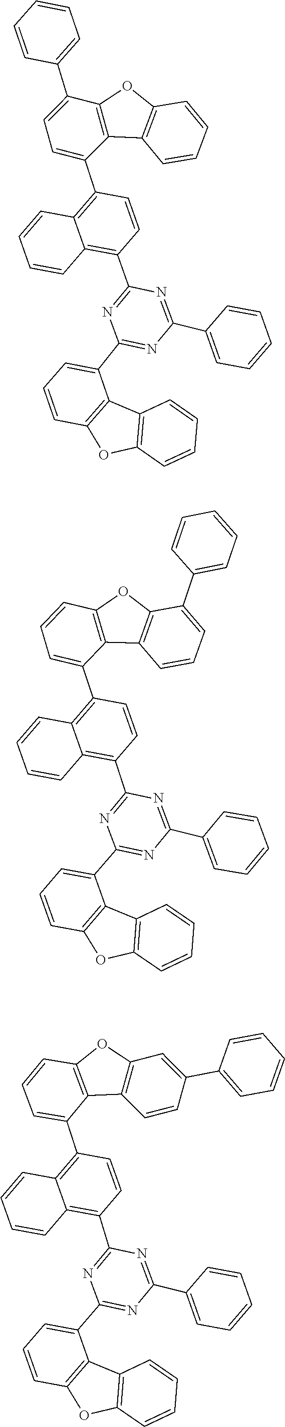 [SMILES: c1ccc(-c2nc(-c3ccc(-c4ccc(-c5ccccc5)c5oc6ccccc6c45)c4ccccc34)nc(-c3cccc4oc5ccccc5c34)n2)cc1 ; c1ccc(-c2nc(-c3ccc(-c4cccc5oc6c(-c7ccccc7)cccc6c45)c4ccccc34)nc(-c3cccc4oc5ccccc5c34)n2)cc1 ; c1ccc(-c2ccc3c(c2)oc2cccc(-c4ccc(-c5nc(-c6ccccc6)nc(-c6cccc7oc8ccccc8c67)n5)c5ccccc45)c23)cc1]

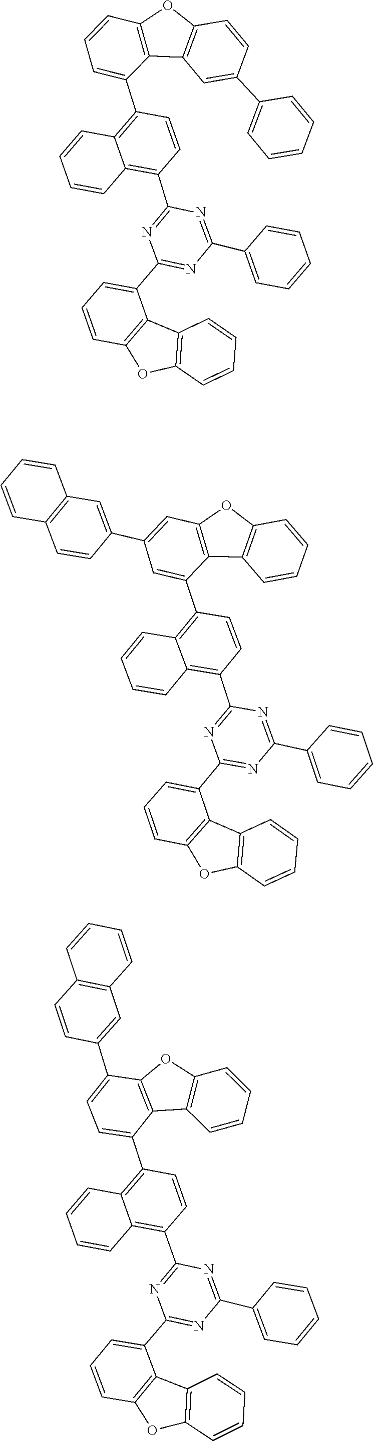 [SMILES: c1ccc(-c2ccc3oc4cccc(-c5ccc(-c6nc(-c7ccccc7)nc(-c7cccc8oc9ccccc9c78)n6)c6ccccc56)c4c3c2)cc1 ; c1ccc(-c2nc(-c3ccc(-c4cc(-c5ccc6ccccc6c5)cc5oc6ccccc6c45)c4ccccc34)nc(-c3cccc4oc5ccccc5c34)n2)cc1 ; c1ccc(-c2nc(-c3ccc(-c4ccc(-c5ccc6ccccc6c5)c5oc6ccccc6c45)c4ccccc34)nc(-c3cccc4oc5ccccc5c34)n2)cc1]

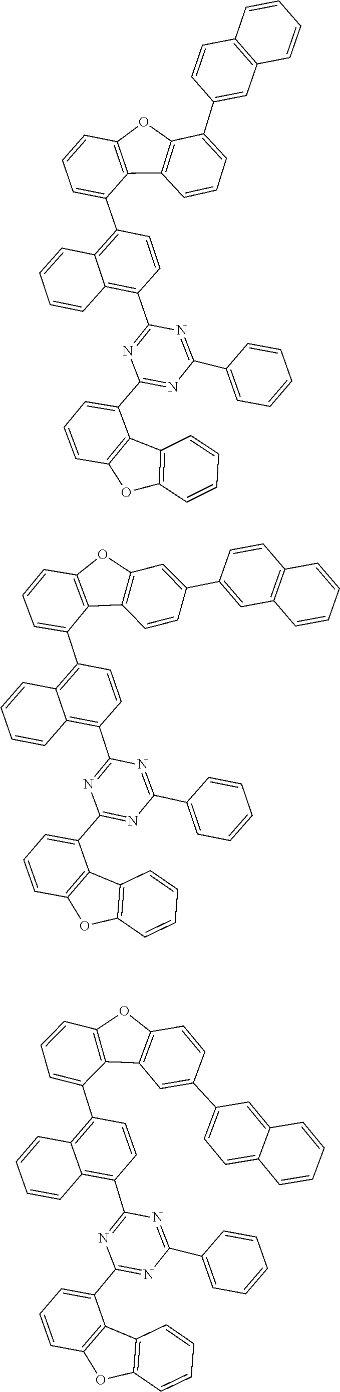 [SMILES: c1ccc(-c2nc(-c3ccc(-c4cccc5oc6c(-c7ccc8ccccc8c7)cccc6c45)c4ccccc34)nc(-c3cccc4oc5ccccc5c34)n2)cc1 ; c1ccc(-c2nc(-c3ccc(-c4cccc5oc6cc(-c7ccc8ccccc8c7)ccc6c45)c4ccccc34)nc(-c3cccc4oc5ccccc5c34)n2)cc1 ; c1ccc(-c2nc(-c3ccc(-c4cccc5oc6ccc(-c7ccc8ccccc8c7)cc6c45)c4ccccc34)nc(-c3cccc4oc5ccccc5c34)n2)cc1]

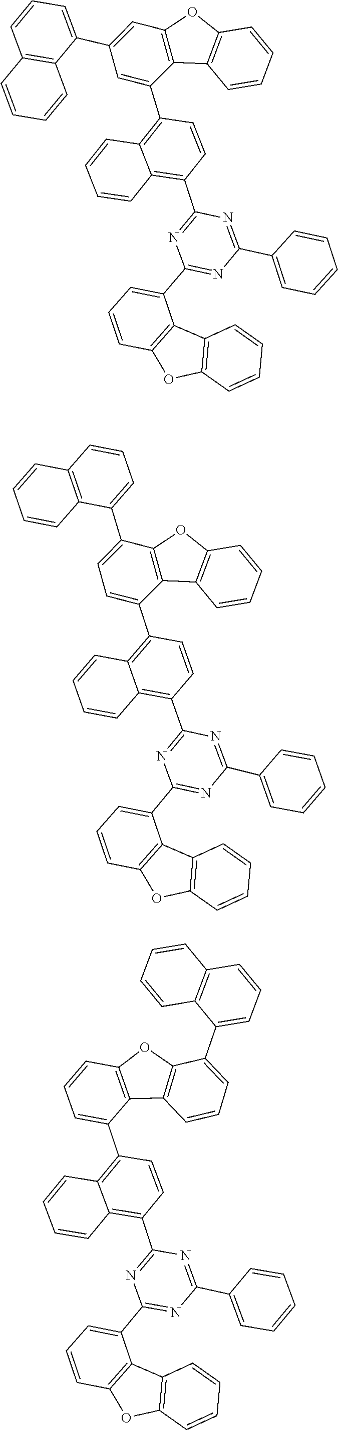

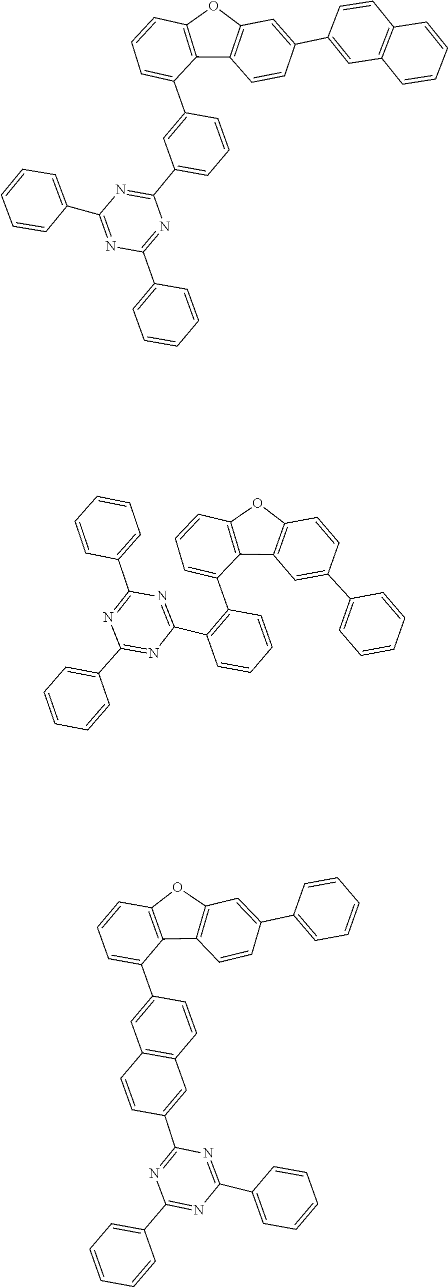 [SMILES: c1ccc(-c2nc(-c3ccccc3)nc(-c3cccc(-c4cccc5oc6cc(-c7ccc8ccccc8c7)ccc6c45)c3)n2)cc1 ; c1ccc(-c2ccc3oc4cccc(-c5ccccc5-c5nc(-c6ccccc6)nc(-c6ccccc6)n5)c4c3c2)cc1 ; c1ccc(-c2ccc3c(c2)oc2cccc(-c4ccc5cc(-c6nc(-c7ccccc7)nc(-c7ccccc7)n6)ccc5c4)c23)cc1]

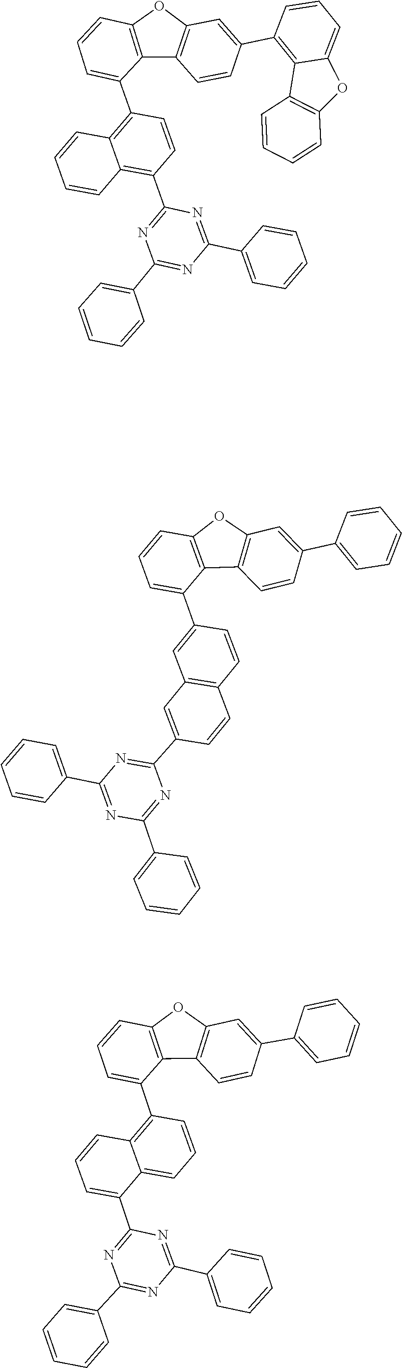 [SMILES: c1ccc(-c2nc(-c3ccccc3)nc(-c3ccc(-c4cccc5oc6cc(-c7cccc8oc9ccccc9c78)ccc6c45)c4ccccc34)n2)cc1 ; c1ccc(-c2ccc3c(c2)oc2cccc(-c4ccc5ccc(-c6nc(-c7ccccc7)nc(-c7ccccc7)n6)cc5c4)c23)cc1 ; c1ccc(-c2ccc3c(c2)oc2cccc(-c4cccc5c(-c6nc(-c7ccccc7)nc(-c7ccccc7)n6)cccc45)c23)cc1]

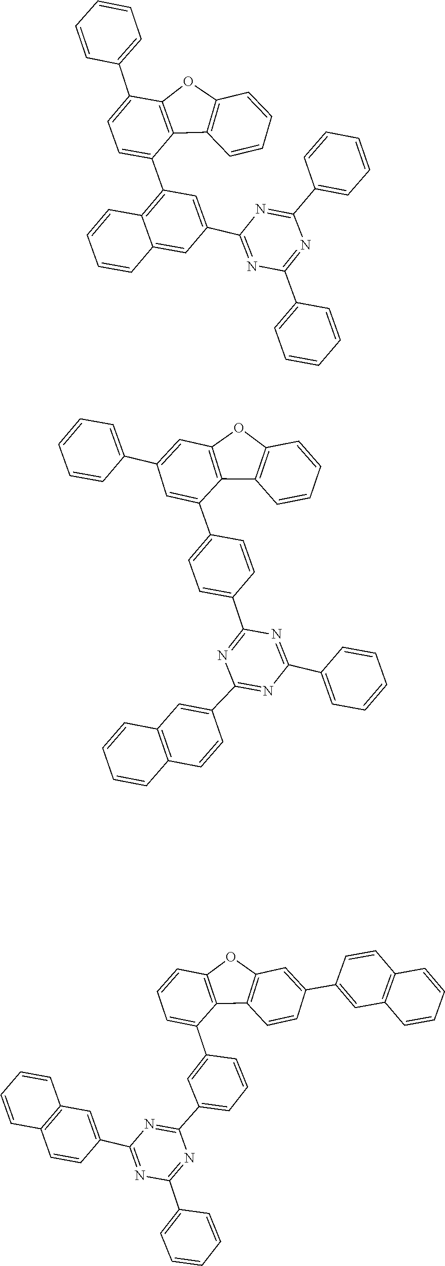 [SMILES: c1ccc(-c2nc(-c3ccccc3)nc(-c3cc(-c4ccc(-c5ccccc5)c5oc6ccccc6c45)c4ccccc4c3)n2)cc1 ; c1ccc(-c2cc(-c3ccc(-c4nc(-c5ccccc5)nc(-c5ccc6ccccc6c5)n4)cc3)c3c(c2)oc2ccccc23)cc1 ; c1ccc(-c2nc(-c3cccc(-c4cccc5oc6cc(-c7ccc8ccccc8c7)ccc6c45)c3)nc(-c3ccc4ccccc4c3)n2)cc1]

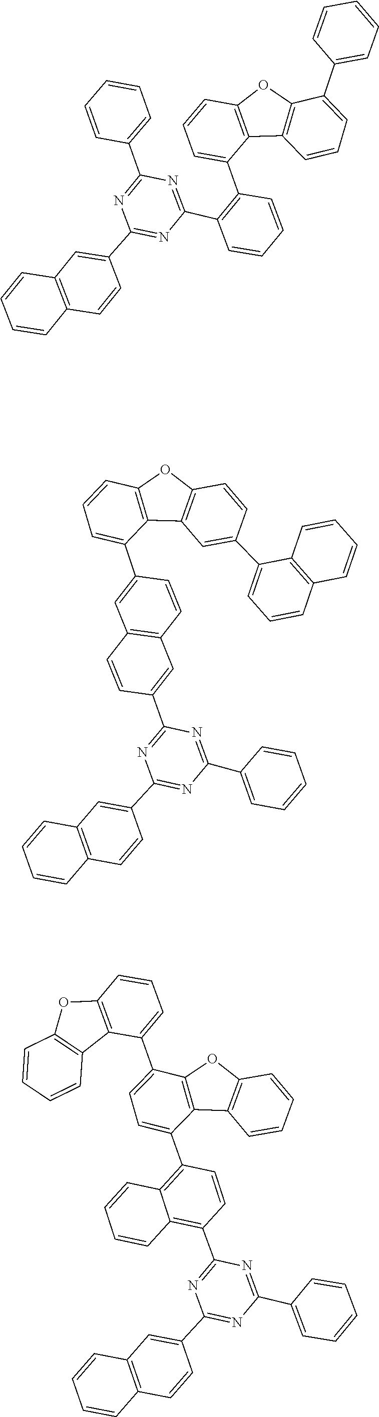 [SMILES: c1ccc(-c2nc(-c3ccc4ccccc4c3)nc(-c3ccccc3-c3cccc4oc5c(-c6ccccc6)cccc5c34)n2)cc1 ; c1ccc(-c2nc(-c3ccc4ccccc4c3)nc(-c3ccc4cc(-c5cccc6oc7ccc(-c8cccc9ccccc89)cc7c56)ccc4c3)n2)cc1 ; c1ccc(-c2nc(-c3ccc4ccccc4c3)nc(-c3ccc(-c4ccc(-c5cccc6oc7ccccc7c56)c5oc6ccccc6c45)c4ccccc34)n2)cc1]

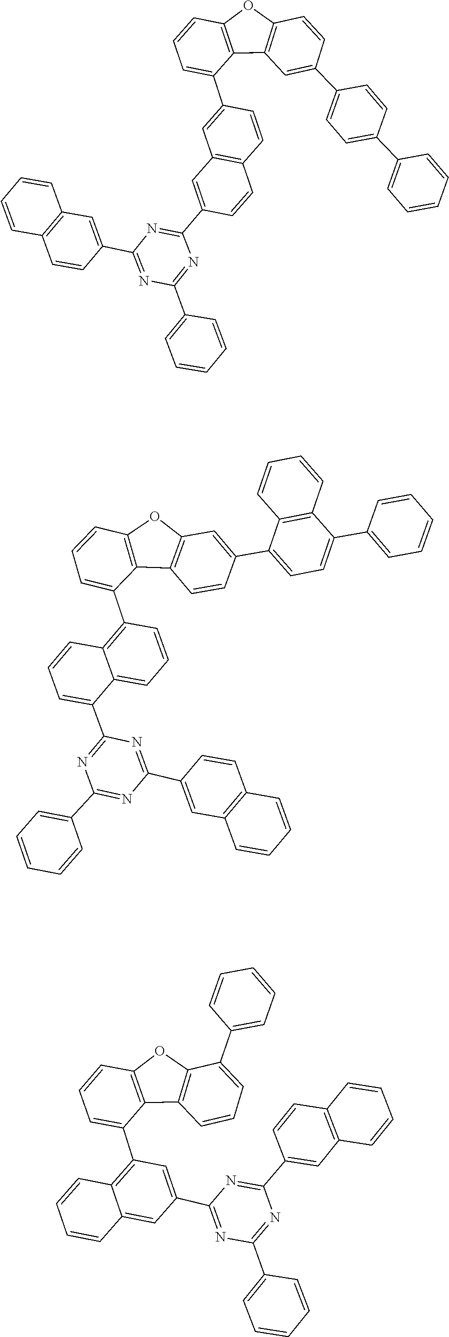 [SMILES: c1ccc(-c2ccc(-c3ccc4oc5cccc(-c6ccc7ccc(-c8nc(-c9ccccc9)nc(-c9ccc%10ccccc%10c9)n8)cc7c6)c5c4c3)cc2)cc1 ; c1ccc(-c2nc(-c3ccc4ccccc4c3)nc(-c3cccc4c(-c5cccc6oc7cc(-c8ccc(-c9ccccc9)c9ccccc89)ccc7c56)cccc34)n2)cc1 ; c1ccc(-c2nc(-c3ccc4ccccc4c3)nc(-c3cc(-c4cccc5oc6c(-c7ccccc7)cccc6c45)c4ccccc4c3)n2)cc1]

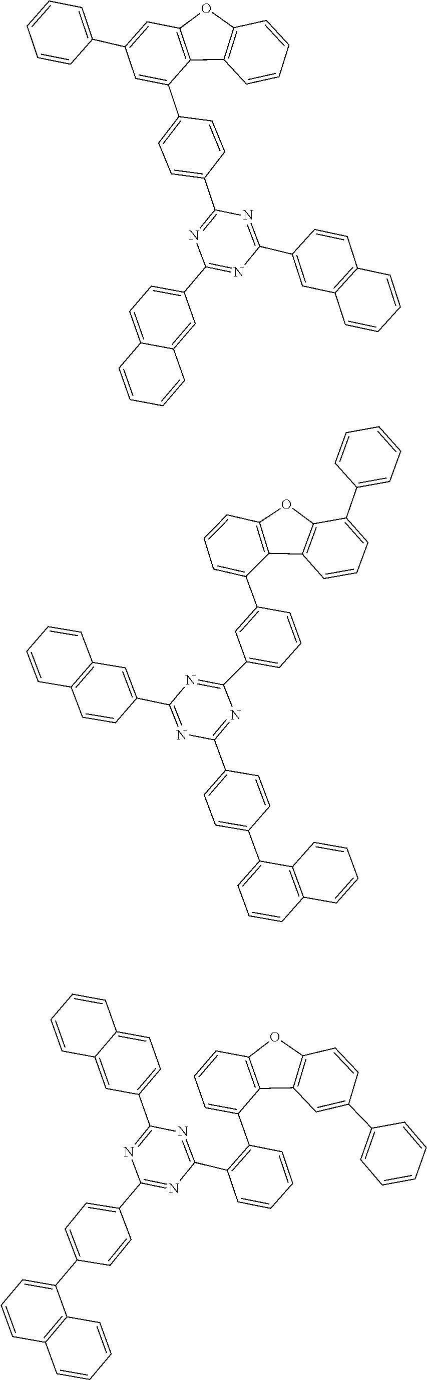 [SMILES: c1ccc(-c2cc(-c3ccc(-c4nc(-c5ccc6ccccc6c5)nc(-c5ccc6ccccc6c5)n4)cc3)c3c(c2)oc2ccccc23)cc1 ; c1ccc(-c2cccc3c2oc2cccc(-c4cccc(-c5nc(-c6ccc(-c7cccc8ccccc78)cc6)nc(-c6ccc7ccccc7c6)n5)c4)c23)cc1 ; c1ccc(-c2ccc3oc4cccc(-c5ccccc5-c5nc(-c6ccc(-c7cccc8ccccc78)cc6)nc(-c6ccc7ccccc7c6)n5)c4c3c2)cc1]

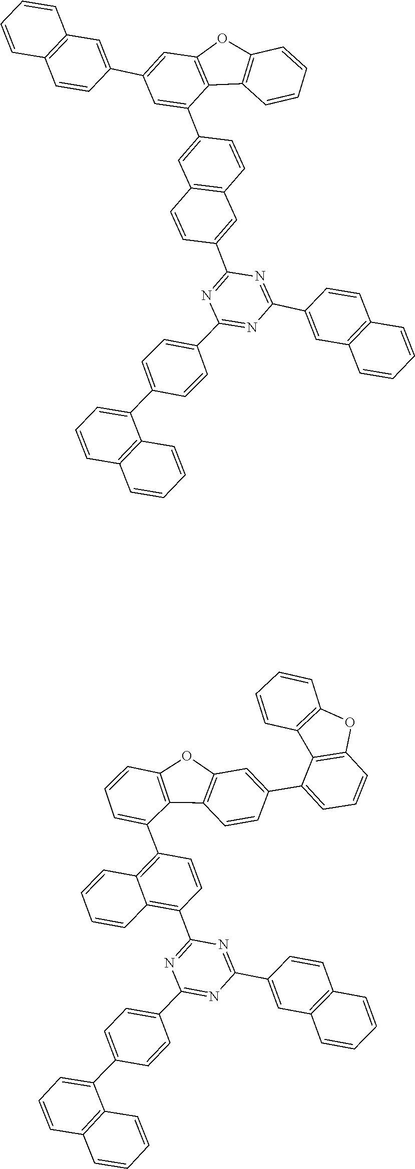 [SMILES: c1ccc2cc(-c3cc(-c4ccc5cc(-c6nc(-c7ccc(-c8cccc9ccccc89)cc7)nc(-c7ccc8ccccc8c7)n6)ccc5c4)c4c(c3)oc3ccccc34)ccc2c1 ; c1ccc2cc(-c3nc(-c4ccc(-c5cccc6ccccc56)cc4)nc(-c4ccc(-c5cccc6oc7cc(-c8cccc9oc%10ccccc%10c89)ccc7c56)c5ccccc45)n3)ccc2c1]

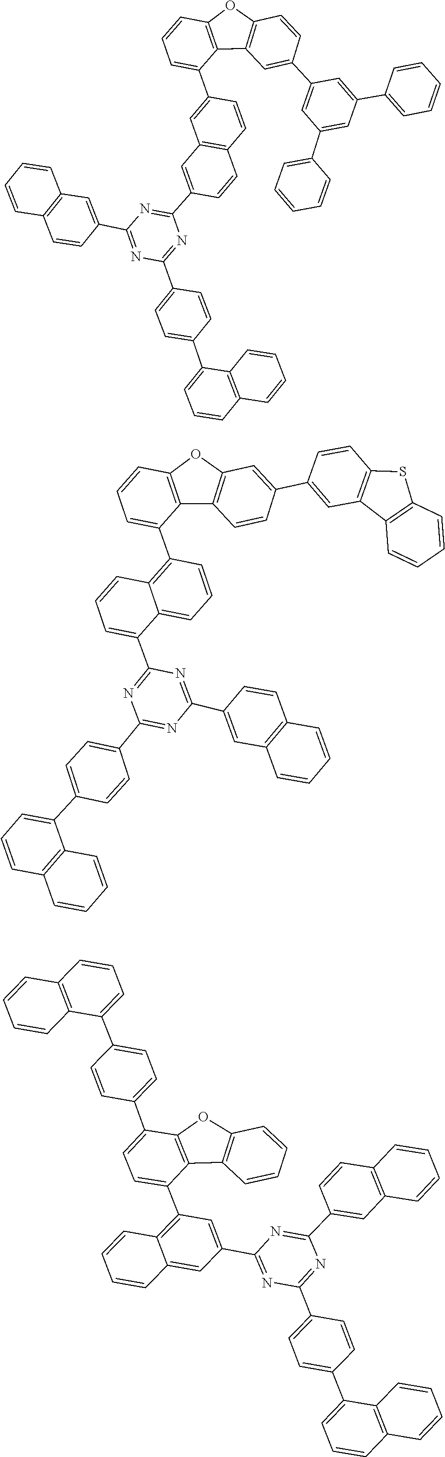

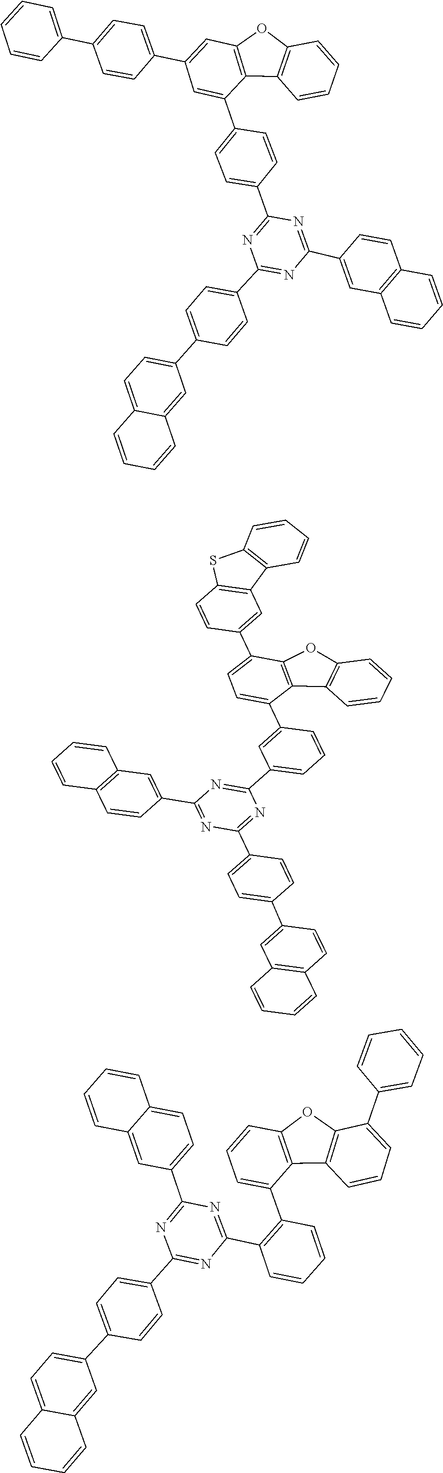 [SMILES: c1ccc(-c2ccc(-c3cc(-c4ccc(-c5nc(-c6ccc(-c7ccc8ccccc8c7)cc6)nc(-c6ccc7ccccc7c6)n5)cc4)c4c(c3)oc3ccccc34)cc2)cc1 ; c1cc(-c2nc(-c3ccc(-c4ccc5ccccc5c4)cc3)nc(-c3ccc4ccccc4c3)n2)cc(-c2ccc(-c3ccc4sc5ccccc5c4c3)c3oc4ccccc4c23)c1 ; c1ccc(-c2cccc3c2oc2cccc(-c4ccccc4-c4nc(-c5ccc(-c6ccc7ccccc7c6)cc5)nc(-c5ccc6ccccc6c5)n4)c23)cc1]

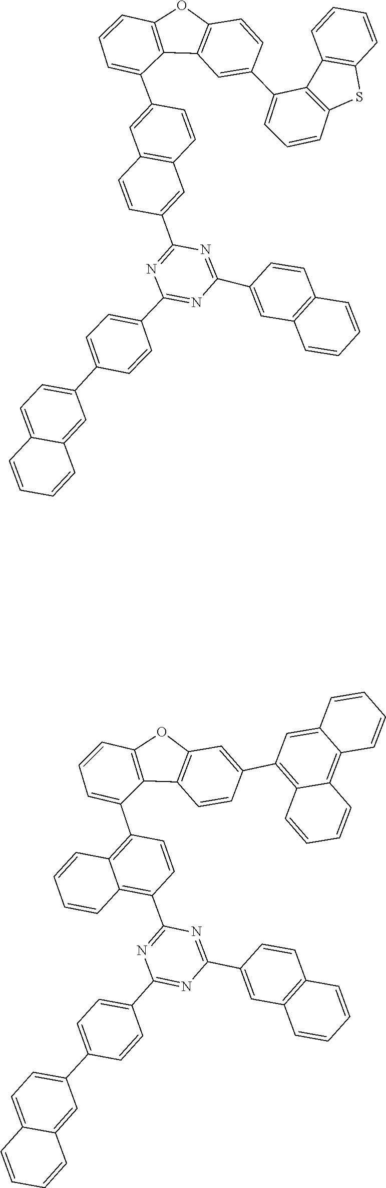 [SMILES: c1ccc2cc(-c3ccc(-c4nc(-c5ccc6ccccc6c5)nc(-c5ccc6cc(-c7cccc8oc9ccc(-c%10cccc%11sc%12ccccc%12c%10%11)cc9c78)ccc6c5)n4)cc3)ccc2c1 ; c1ccc2cc(-c3ccc(-c4nc(-c5ccc6ccccc6c5)nc(-c5ccc(-c6cccc7oc8cc(-c9cc%10ccccc%10c%10ccccc9%10)ccc8c67)c6ccccc56)n4)cc3)ccc2c1]

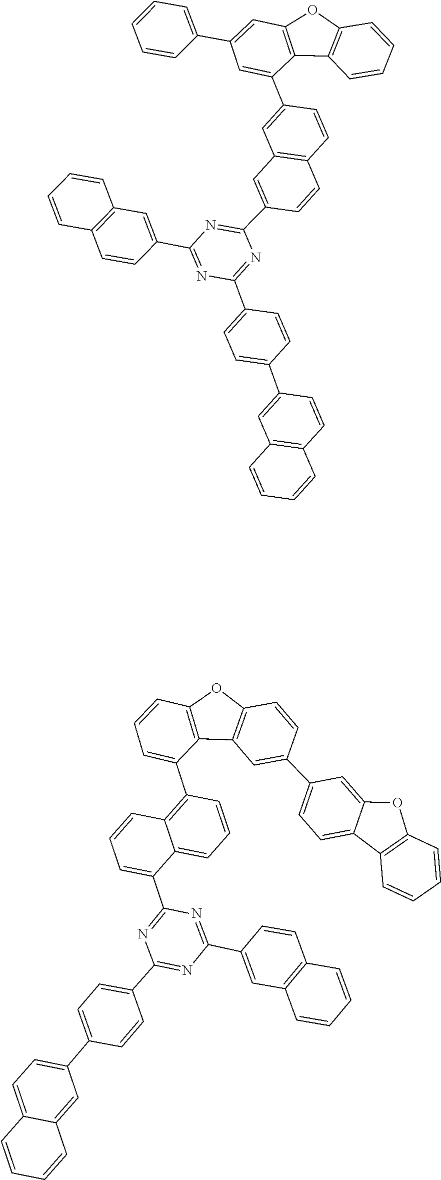 [SMILES: c1ccc(-c2cc(-c3ccc4ccc(-c5nc(-c6ccc(-c7ccc8ccccc8c7)cc6)nc(-c6ccc7ccccc7c6)n5)cc4c3)c3c(c2)oc2ccccc23)cc1 ; c1ccc2cc(-c3ccc(-c4nc(-c5ccc6ccccc6c5)nc(-c5cccc6c(-c7cccc8oc9ccc(-c%10ccc%11c(c%10)oc%10ccccc%10%11)cc9c78)cccc56)n4)cc3)ccc2c1]

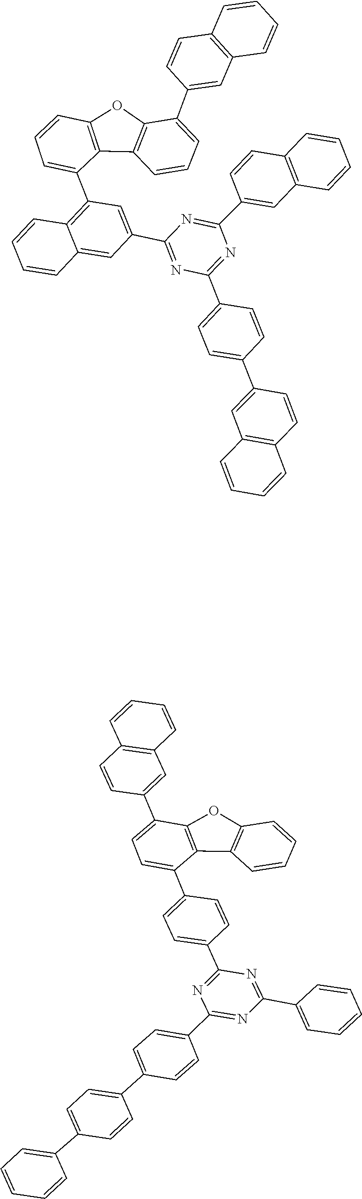 [SMILES: c1ccc2cc(-c3ccc(-c4nc(-c5ccc6ccccc6c5)nc(-c5cc(-c6cccc7oc8c(-c9ccc%10ccccc%10c9)cccc8c67)c6ccccc6c5)n4)cc3)ccc2c1 ; c1ccc(-c2ccc(-c3ccc(-c4nc(-c5ccccc5)nc(-c5ccc(-c6ccc(-c7ccc8ccccc8c7)c7oc8ccccc8c67)cc5)n4)cc3)cc2)cc1]

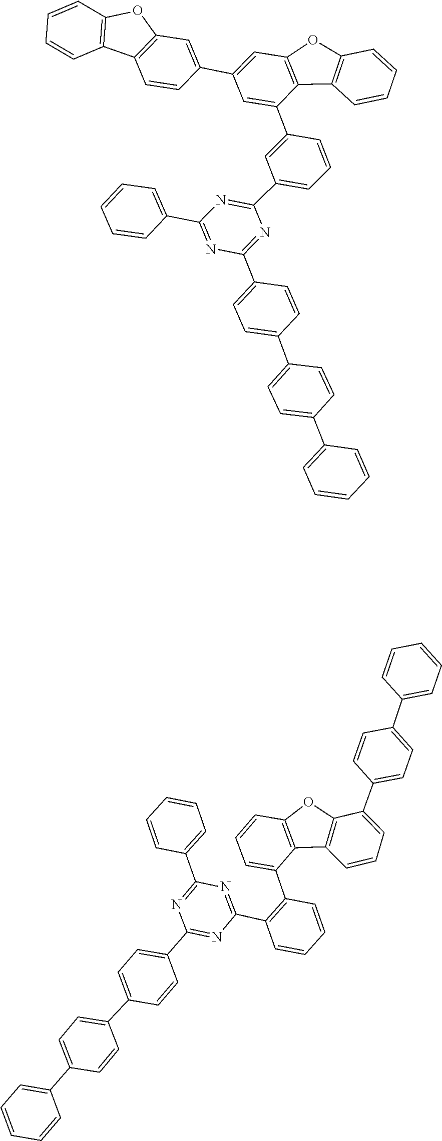 [SMILES: c1ccc(-c2ccc(-c3ccc(-c4nc(-c5ccccc5)nc(-c5cccc(-c6cc(-c7ccc8c(c7)oc7ccccc78)cc7oc8ccccc8c67)c5)n4)cc3)cc2)cc1 ; c1ccc(-c2ccc(-c3ccc(-c4nc(-c5ccccc5)nc(-c5ccccc5-c5cccc6oc7c(-c8ccc(-c9ccccc9)cc8)cccc7c56)n4)cc3)cc2)cc1]

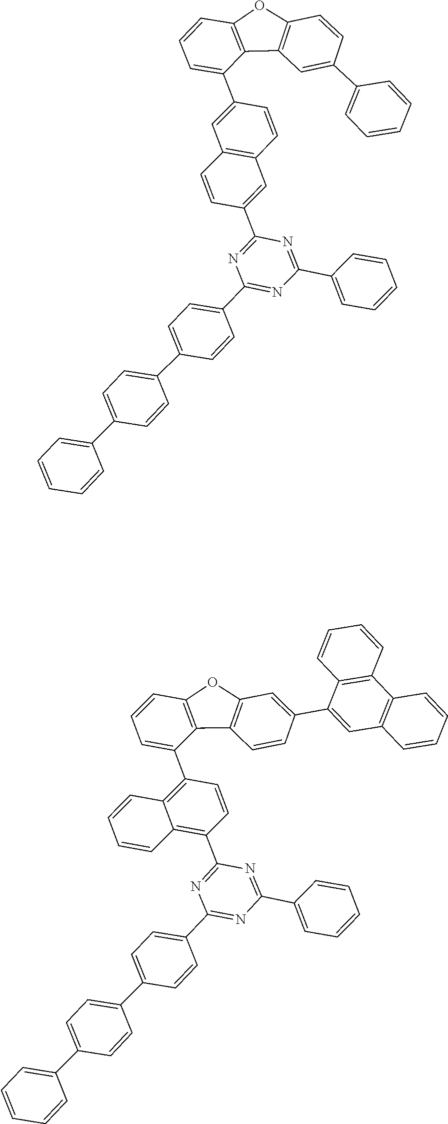 [SMILES: c1ccc(-c2ccc(-c3ccc(-c4nc(-c5ccccc5)nc(-c5ccc6cc(-c7cccc8oc9ccc(-c%10ccccc%10)cc9c78)ccc6c5)n4)cc3)cc2)cc1 ; c1ccc(-c2ccc(-c3ccc(-c4nc(-c5ccccc5)nc(-c5ccc(-c6cccc7oc8cc(-c9cc%10ccccc%10c%10ccccc9%10)ccc8c67)c6ccccc56)n4)cc3)cc2)cc1]

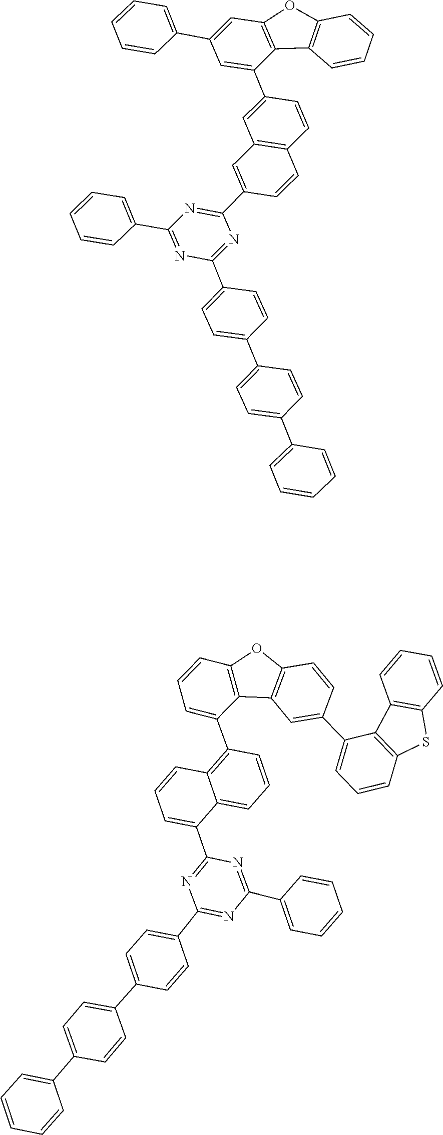 [SMILES: c1ccc(-c2ccc(-c3ccc(-c4nc(-c5ccccc5)nc(-c5ccc6ccc(-c7cc(-c8ccccc8)cc8oc9ccccc9c78)cc6c5)n4)cc3)cc2)cc1 ; c1ccc(-c2ccc(-c3ccc(-c4nc(-c5ccccc5)nc(-c5cccc6c(-c7cccc8oc9ccc(-c%10cccc%11sc%12ccccc%12c%10%11)cc9c78)cccc56)n4)cc3)cc2)cc1]

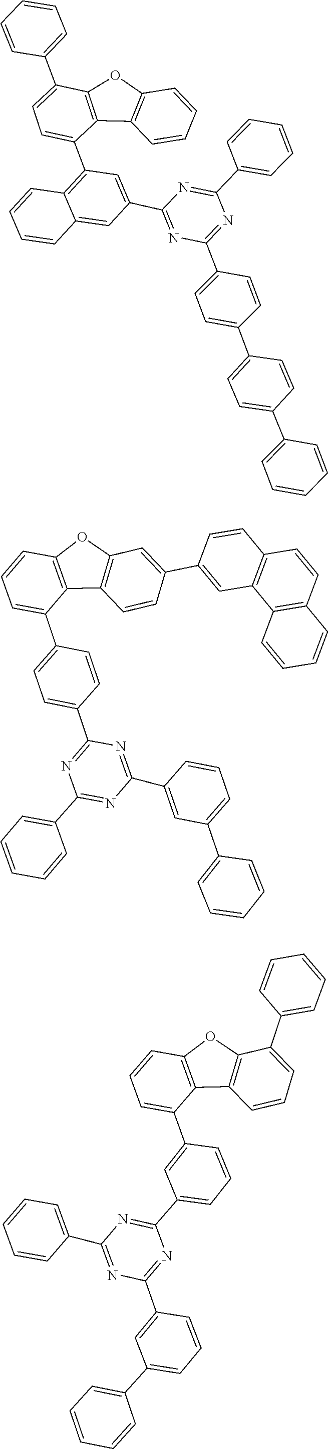 [SMILES: c1ccc(-c2ccc(-c3ccc(-c4nc(-c5ccccc5)nc(-c5cc(-c6ccc(-c7ccccc7)c7oc8ccccc8c67)c6ccccc6c5)n4)cc3)cc2)cc1 ; c1ccc(-c2cccc(-c3nc(-c4ccccc4)nc(-c4ccc(-c5cccc6oc7cc(-c8ccc9ccc%10ccccc%10c9c8)ccc7c56)cc4)n3)c2)cc1 ; c1ccc(-c2cccc(-c3nc(-c4ccccc4)nc(-c4cccc(-c5cccc6oc7c(-c8ccccc8)cccc7c56)c4)n3)c2)cc1]

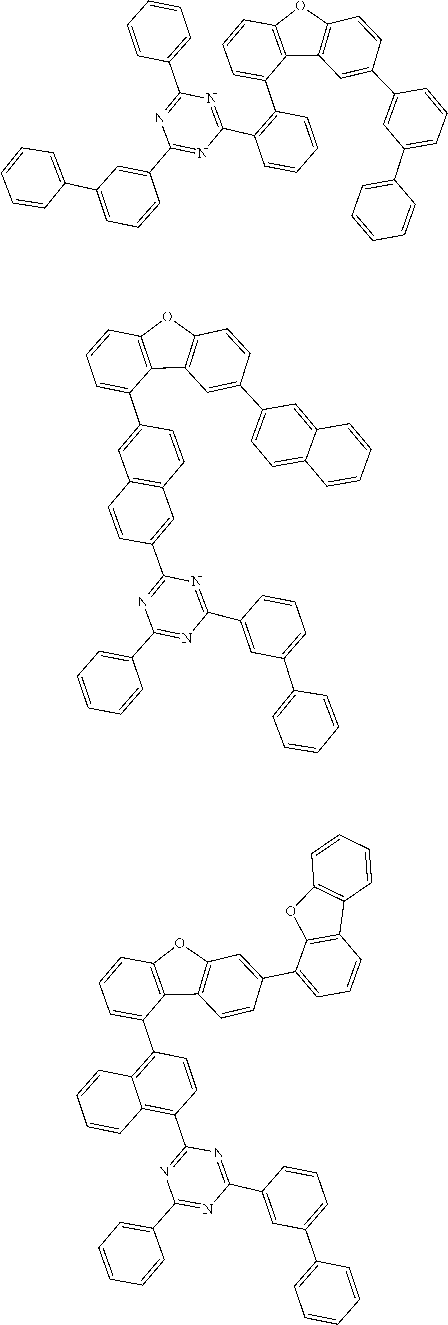 [SMILES: c1ccc(-c2cccc(-c3ccc4oc5cccc(-c6ccccc6-c6nc(-c7ccccc7)nc(-c7cccc(-c8ccccc8)c7)n6)c5c4c3)c2)cc1 ; c1ccc(-c2cccc(-c3nc(-c4ccccc4)nc(-c4ccc5cc(-c6cccc7oc8ccc(-c9ccc%10ccccc%10c9)cc8c67)ccc5c4)n3)c2)cc1 ; c1ccc(-c2cccc(-c3nc(-c4ccccc4)nc(-c4ccc(-c5cccc6oc7cc(-c8cccc9c8oc8ccccc89)ccc7c56)c5ccccc45)n3)c2)cc1]

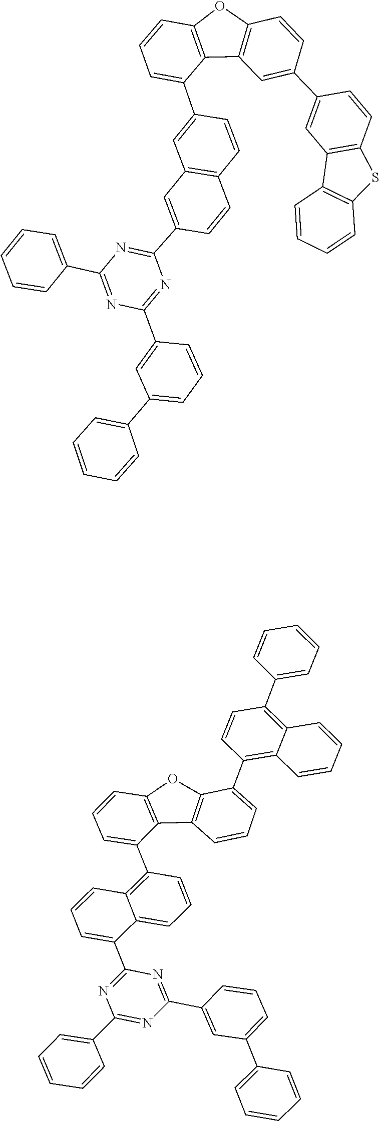 [SMILES: c1ccc(-c2cccc(-c3nc(-c4ccccc4)nc(-c4ccc5ccc(-c6cccc7oc8ccc(-c9ccc%10sc%11ccccc%11c%10c9)cc8c67)cc5c4)n3)c2)cc1 ; c1ccc(-c2cccc(-c3nc(-c4ccccc4)nc(-c4cccc5c(-c6cccc7oc8c(-c9ccc(-c%10ccccc%10)c%10ccccc9%10)cccc8c67)cccc45)n3)c2)cc1]

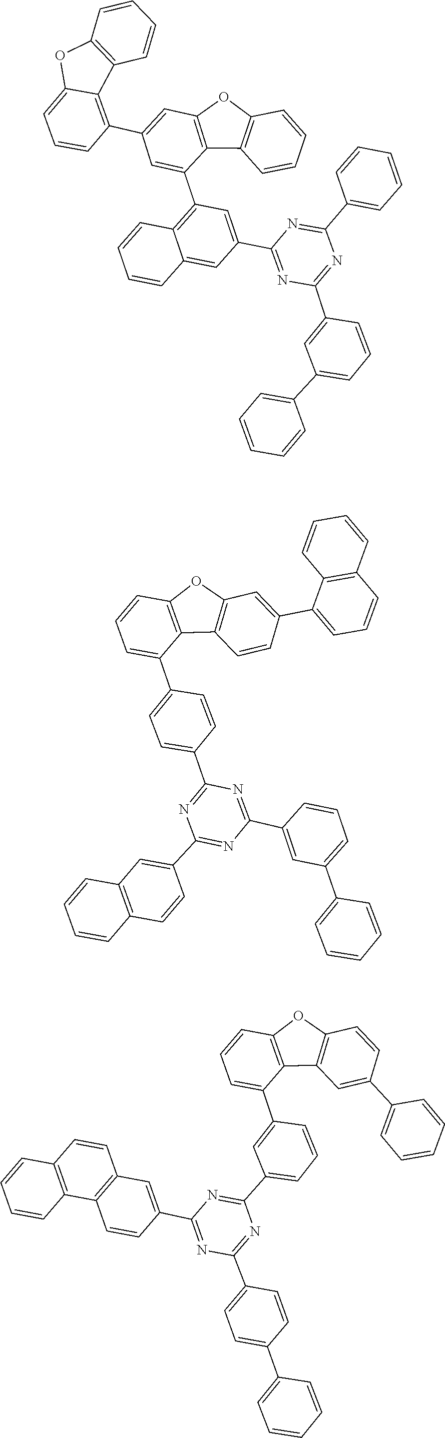 [SMILES: c1ccc(-c2cccc(-c3nc(-c4ccccc4)nc(-c4cc(-c5cc(-c6cccc7oc8ccccc8c67)cc6oc7ccccc7c56)c5ccccc5c4)n3)c2)cc1 ; c1ccc(-c2cccc(-c3nc(-c4ccc(-c5cccc6oc7cc(-c8cccc9ccccc89)ccc7c56)cc4)nc(-c4ccc5ccccc5c4)n3)c2)cc1 ; c1ccc(-c2ccc(-c3nc(-c4cccc(-c5cccc6oc7ccc(-c8ccccc8)cc7c56)c4)nc(-c4ccc5c(ccc6ccccc65)c4)n3)cc2)cc1]

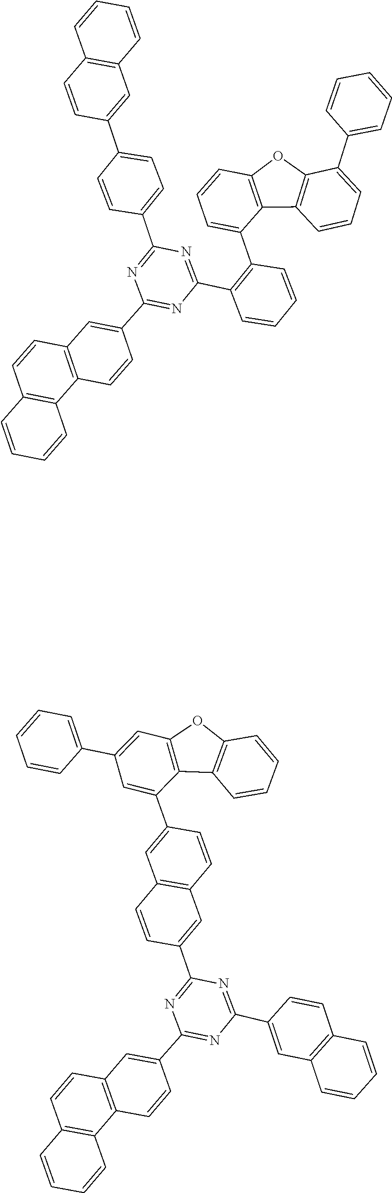 [SMILES: c1ccc(-c2cccc3c2oc2cccc(-c4ccccc4-c4nc(-c5ccc(-c6ccc7ccccc7c6)cc5)nc(-c5ccc6c(ccc7ccccc76)c5)n4)c23)cc1 ; c1ccc(-c2cc(-c3ccc4cc(-c5nc(-c6ccc7ccccc7c6)nc(-c6ccc7c(ccc8ccccc87)c6)n5)ccc4c3)c3c(c2)oc2ccccc23)cc1]

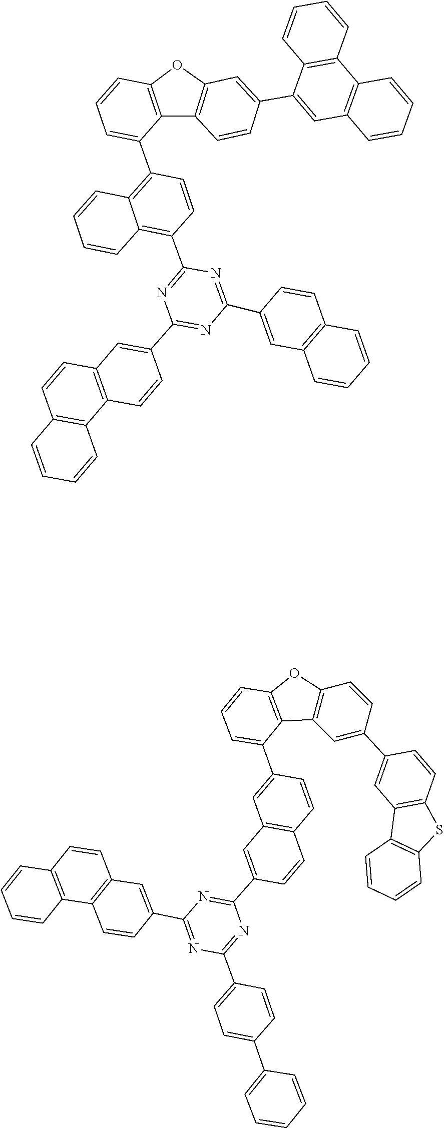 [SMILES: c1ccc2cc(-c3nc(-c4ccc5c(ccc6ccccc65)c4)nc(-c4ccc(-c5cccc6oc7cc(-c8cc9ccccc9c9ccccc89)ccc7c56)c5ccccc45)n3)ccc2c1 ; c1ccc(-c2ccc(-c3nc(-c4ccc5ccc(-c6cccc7oc8ccc(-c9ccc%10sc%11ccccc%11c%10c9)cc8c67)cc5c4)nc(-c4ccc5c(ccc6ccccc65)c4)n3)cc2)cc1]

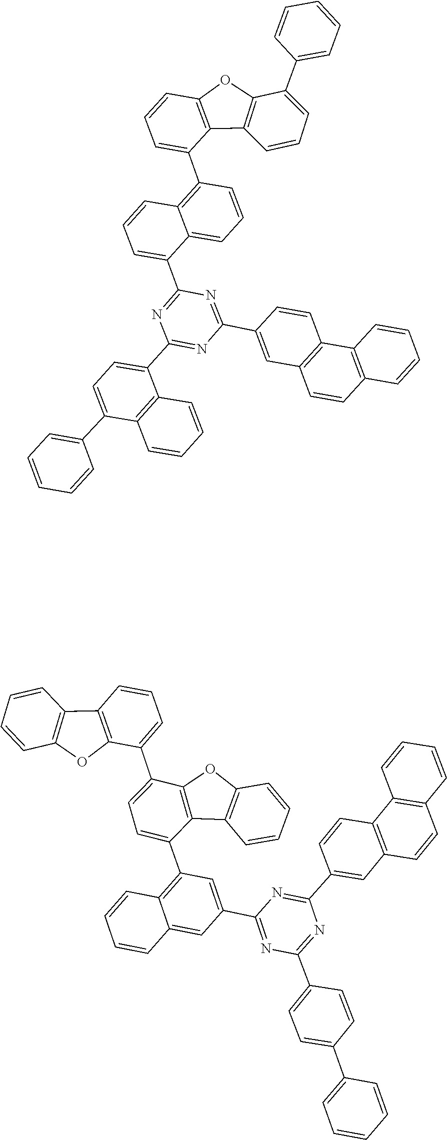 [SMILES: c1ccc(-c2ccc(-c3nc(-c4ccc5c(ccc6ccccc65)c4)nc(-c4cccc5c(-c6cccc7oc8c(-c9ccccc9)cccc8c67)cccc45)n3)c3ccccc23)cc1 ; c1ccc(-c2ccc(-c3nc(-c4cc(-c5ccc(-c6cccc7c6oc6ccccc67)c6oc7ccccc7c56)c5ccccc5c4)nc(-c4ccc5c(ccc6ccccc65)c4)n3)cc2)cc1]

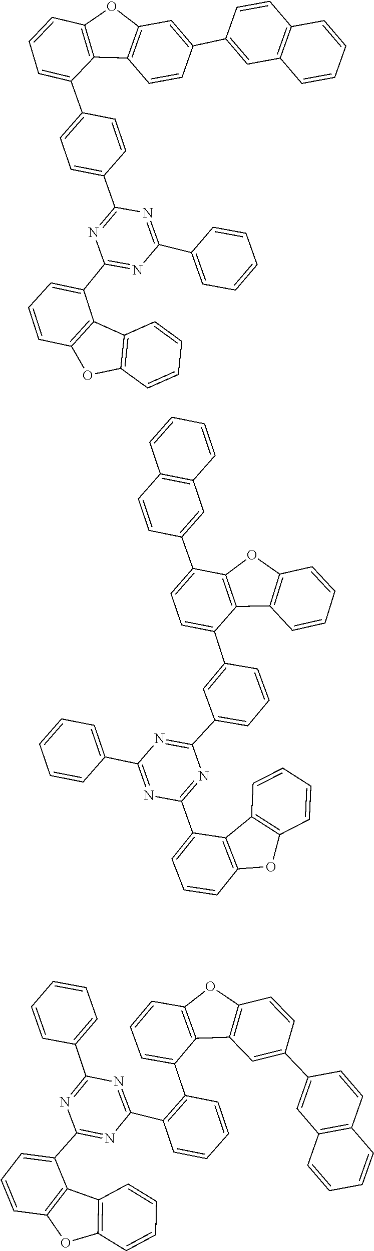

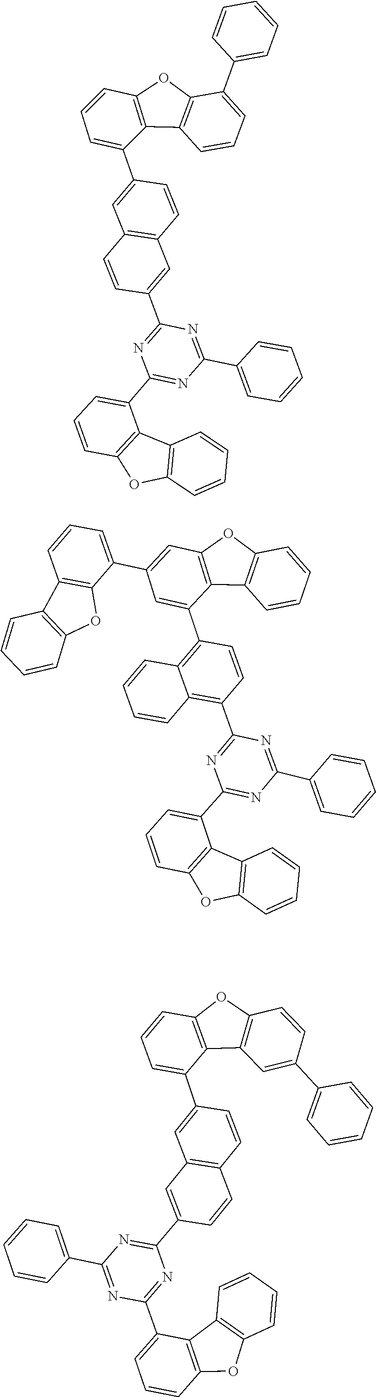 [SMILES: c1ccc(-c2nc(-c3ccc4cc(-c5cccc6oc7c(-c8ccccc8)cccc7c56)ccc4c3)nc(-c3cccc4oc5ccccc5c34)n2)cc1 ; c1ccc(-c2nc(-c3ccc(-c4cc(-c5cccc6c5oc5ccccc56)cc5oc6ccccc6c45)c4ccccc34)nc(-c3cccc4oc5ccccc5c34)n2)cc1 ; c1ccc(-c2ccc3oc4cccc(-c5ccc6ccc(-c7nc(-c8ccccc8)nc(-c8cccc9oc%10ccccc%10c89)n7)cc6c5)c4c3c2)cc1]

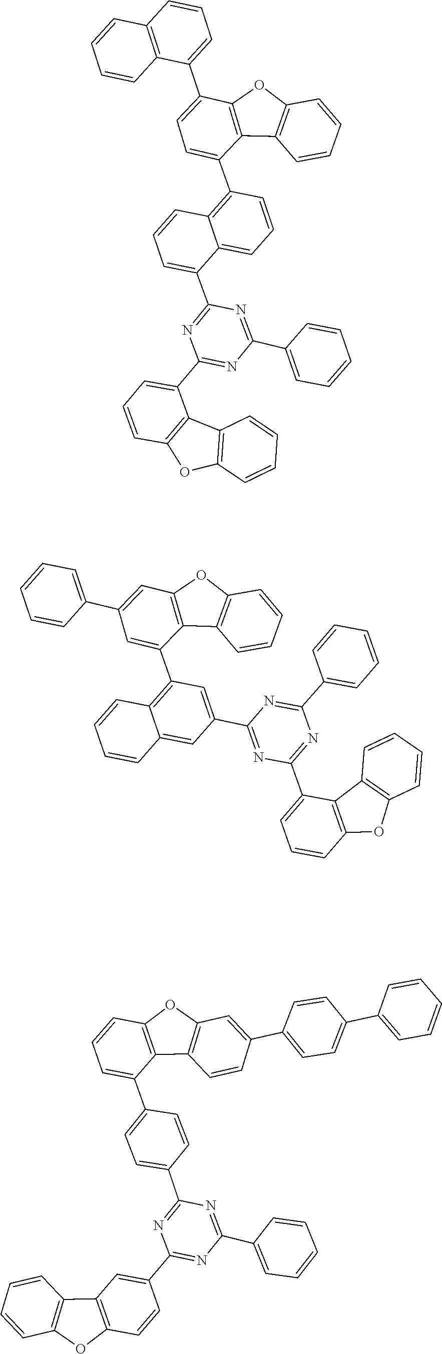 [SMILES: c1ccc(-c2nc(-c3cccc4c(-c5ccc(-c6cccc7ccccc67)c6oc7ccccc7c56)cccc34)nc(-c3cccc4oc5ccccc5c34)n2)cc1 ; c1ccc(-c2cc(-c3cc(-c4nc(-c5ccccc5)nc(-c5cccc6oc7ccccc7c56)n4)cc4ccccc34)c3c(c2)oc2ccccc23)cc1 ; c1ccc(-c2ccc(-c3ccc4c(c3)oc3cccc(-c5ccc(-c6nc(-c7ccccc7)nc(-c7ccc8oc9ccccc9c8c7)n6)cc5)c34)cc2)cc1]

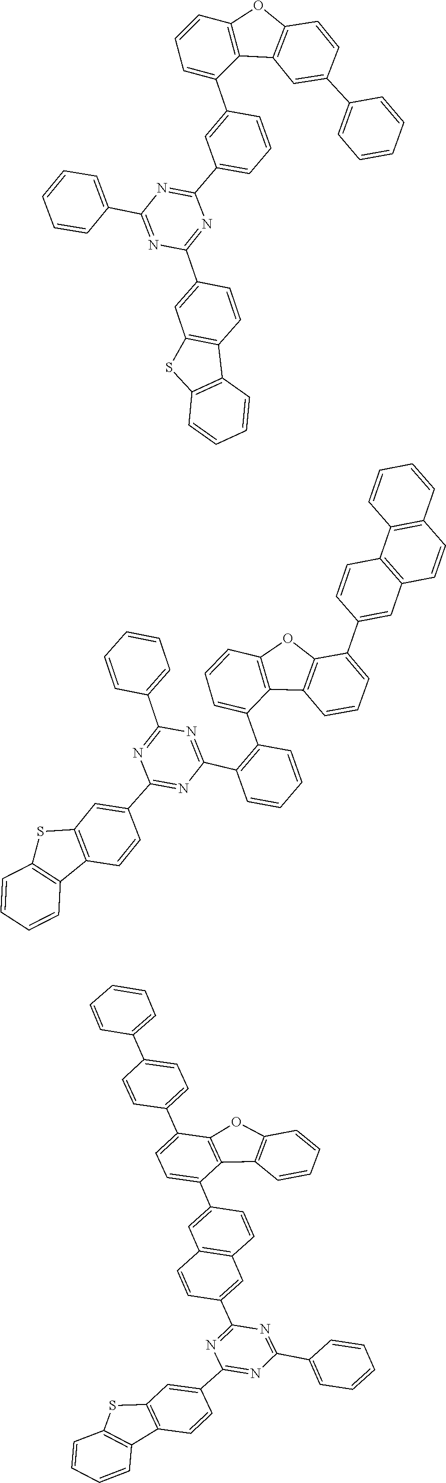 [SMILES: c1ccc(-c2ccc3oc4cccc(-c5cccc(-c6nc(-c7ccccc7)nc(-c7ccc8c(c7)sc7ccccc78)n6)c5)c4c3c2)cc1 ; c1ccc(-c2nc(-c3ccc4c(c3)sc3ccccc34)nc(-c3ccccc3-c3cccc4oc5c(-c6ccc7c(ccc8ccccc87)c6)cccc5c34)n2)cc1 ; c1ccc(-c2ccc(-c3ccc(-c4ccc5cc(-c6nc(-c7ccccc7)nc(-c7ccc8c(c7)sc7ccccc78)n6)ccc5c4)c4c3oc3ccccc34)cc2)cc1]

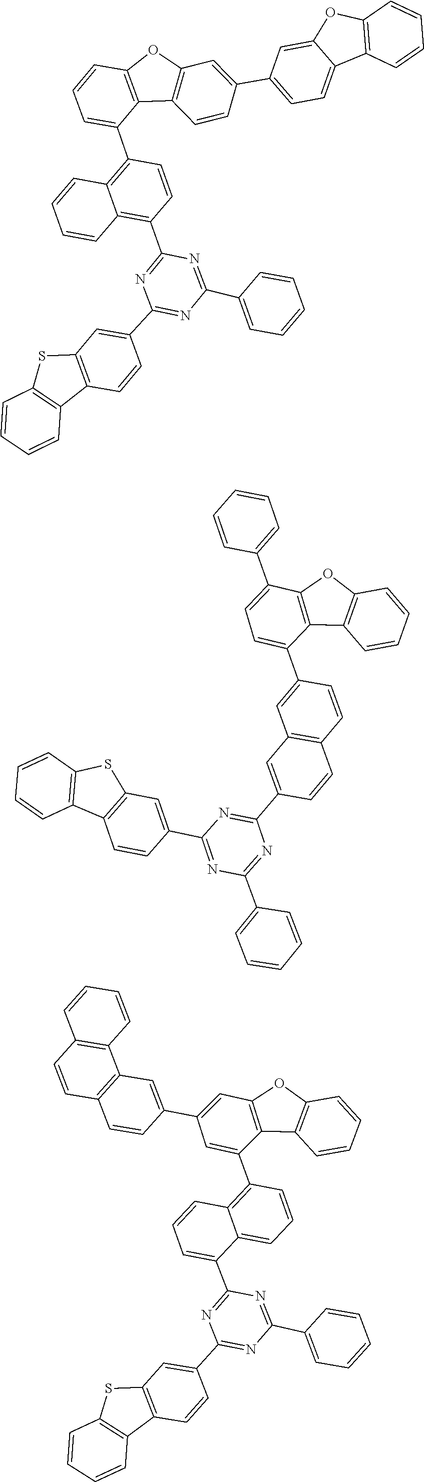 [SMILES: c1ccc(-c2nc(-c3ccc4c(c3)sc3ccccc34)nc(-c3ccc(-c4cccc5oc6cc(-c7ccc8c(c7)oc7ccccc78)ccc6c45)c4ccccc34)n2)cc1 ; c1ccc(-c2nc(-c3ccc4ccc(-c5ccc(-c6ccccc6)c6oc7ccccc7c56)cc4c3)nc(-c3ccc4c(c3)sc3ccccc34)n2)cc1 ; c1ccc(-c2nc(-c3ccc4c(c3)sc3ccccc34)nc(-c3cccc4c(-c5cc(-c6ccc7ccc8ccccc8c7c6)cc6oc7ccccc7c56)cccc34)n2)cc1]

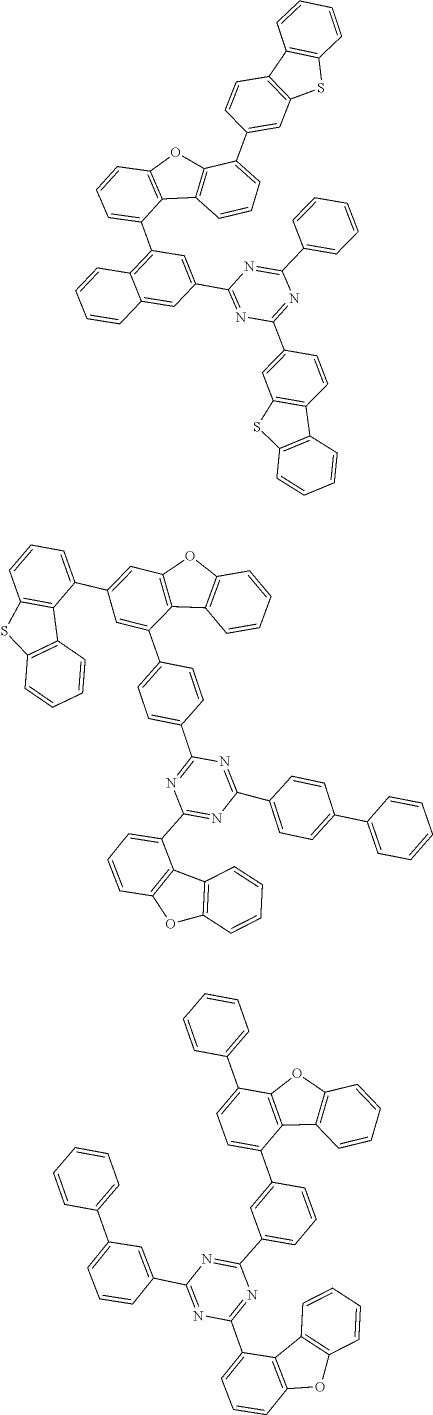 [SMILES: c1ccc(-c2nc(-c3cc(-c4cccc5oc6c(-c7ccc8c(c7)sc7ccccc78)cccc6c45)c4ccccc4c3)nc(-c3ccc4c(c3)sc3ccccc34)n2)cc1 ; c1ccc(-c2ccc(-c3nc(-c4ccc(-c5cc(-c6cccc7sc8ccccc8c67)cc6oc7ccccc7c56)cc4)nc(-c4cccc5oc6ccccc6c45)n3)cc2)cc1 ; c1ccc(-c2cccc(-c3nc(-c4cccc(-c5ccc(-c6ccccc6)c6oc7ccccc7c56)c4)nc(-c4cccc5oc6ccccc6c45)n3)c2)cc1]

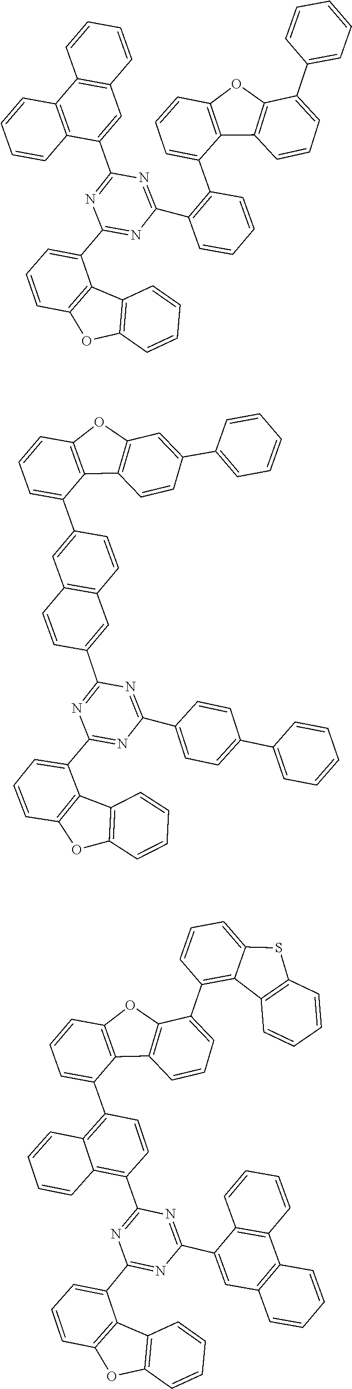 [SMILES: c1ccc(-c2cccc3c2oc2cccc(-c4ccccc4-c4nc(-c5cc6ccccc6c6ccccc56)nc(-c5cccc6oc7ccccc7c56)n4)c23)cc1 ; c1ccc(-c2ccc(-c3nc(-c4ccc5cc(-c6cccc7oc8cc(-c9ccccc9)ccc8c67)ccc5c4)nc(-c4cccc5oc6ccccc6c45)n3)cc2)cc1 ; c1ccc2c(c1)cc(-c1nc(-c3ccc(-c4cccc5oc6c(-c7cccc8sc9ccccc9c78)cccc6c45)c4ccccc34)nc(-c3cccc4oc5ccccc5c34)n1)c1ccccc12]

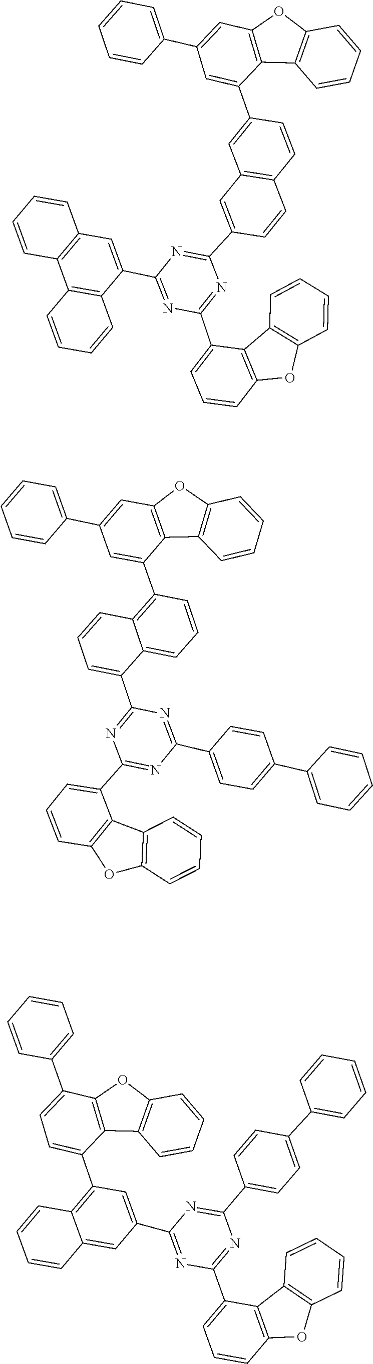 [SMILES: c1ccc(-c2cc(-c3ccc4ccc(-c5nc(-c6cc7ccccc7c7ccccc67)nc(-c6cccc7oc8ccccc8c67)n5)cc4c3)c3c(c2)oc2ccccc23)cc1 ; c1ccc(-c2ccc(-c3nc(-c4cccc5c(-c6cc(-c7ccccc7)cc7oc8ccccc8c67)cccc45)nc(-c4cccc5oc6ccccc6c45)n3)cc2)cc1 ; c1ccc(-c2ccc(-c3nc(-c4cc(-c5ccc(-c6ccccc6)c6oc7ccccc7c56)c5ccccc5c4)nc(-c4cccc5oc6ccccc6c45)n3)cc2)cc1]

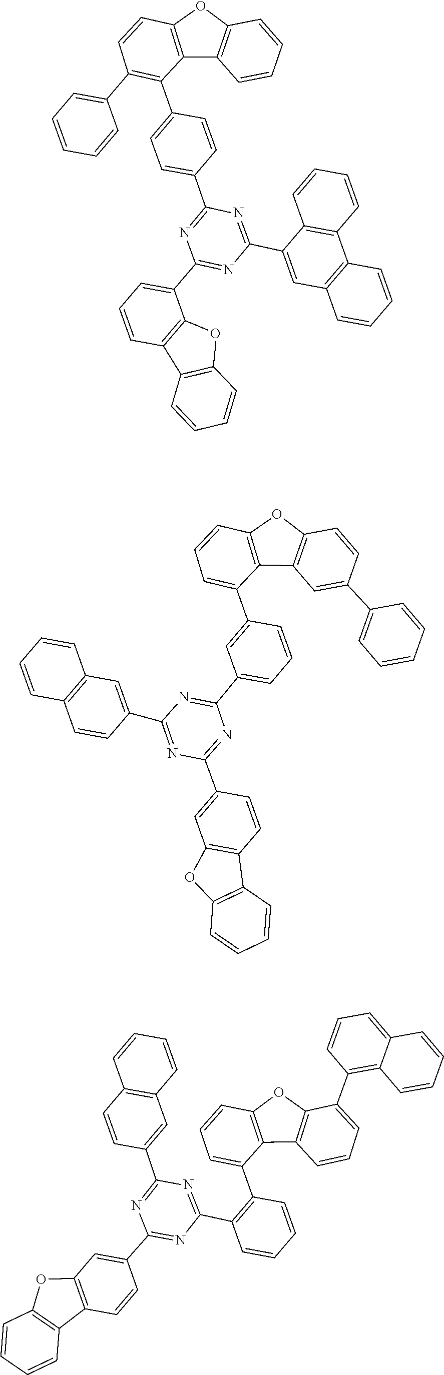 [SMILES: c1ccc(-c2ccc3oc4ccccc4c3c2-c2ccc(-c3nc(-c4cc5ccccc5c5ccccc45)nc(-c4cccc5c4oc4ccccc45)n3)cc2)cc1 ; c1ccc(-c2ccc3oc4cccc(-c5cccc(-c6nc(-c7ccc8ccccc8c7)nc(-c7ccc8c(c7)oc7ccccc78)n6)c5)c4c3c2)cc1 ; c1ccc(-c2cccc3oc4c(-c5cccc6ccccc56)cccc4c23)c(-c2nc(-c3ccc4ccccc4c3)nc(-c3ccc4c(c3)oc3ccccc34)n2)c1]

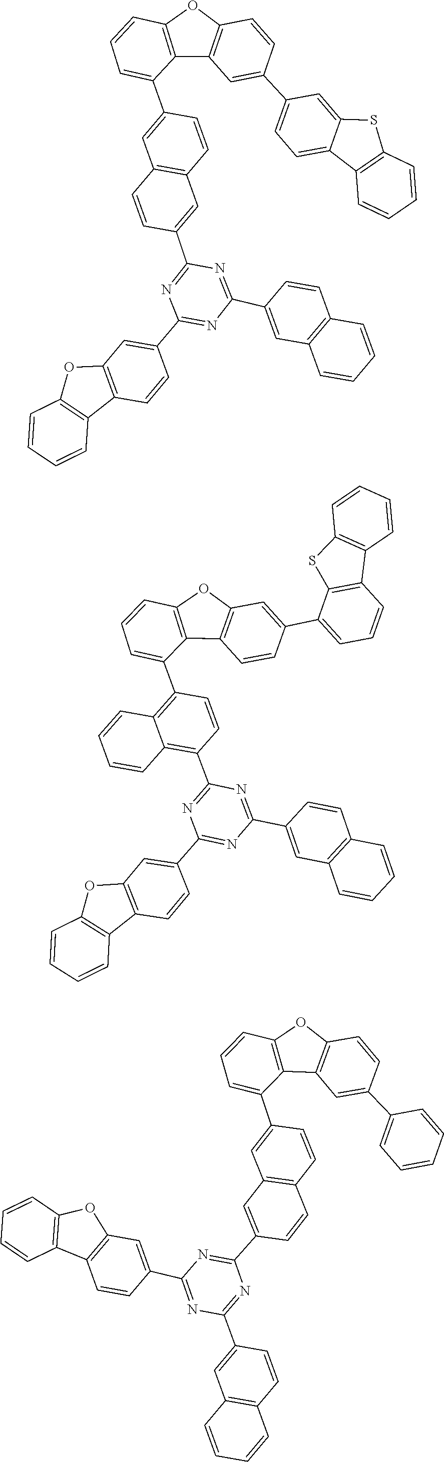 [SMILES: c1ccc2cc(-c3nc(-c4ccc5cc(-c6cccc7oc8ccc(-c9ccc%10c(c9)sc9ccccc9%10)cc8c67)ccc5c4)nc(-c4ccc5c(c4)oc4ccccc45)n3)ccc2c1 ; c1ccc2cc(-c3nc(-c4ccc5c(c4)oc4ccccc45)nc(-c4ccc(-c5cccc6oc7cc(-c8cccc9c8sc8ccccc89)ccc7c56)c5ccccc45)n3)ccc2c1 ; c1ccc(-c2ccc3oc4cccc(-c5ccc6ccc(-c7nc(-c8ccc9ccccc9c8)nc(-c8ccc9c(c8)oc8ccccc89)n7)cc6c5)c4c3c2)cc1]

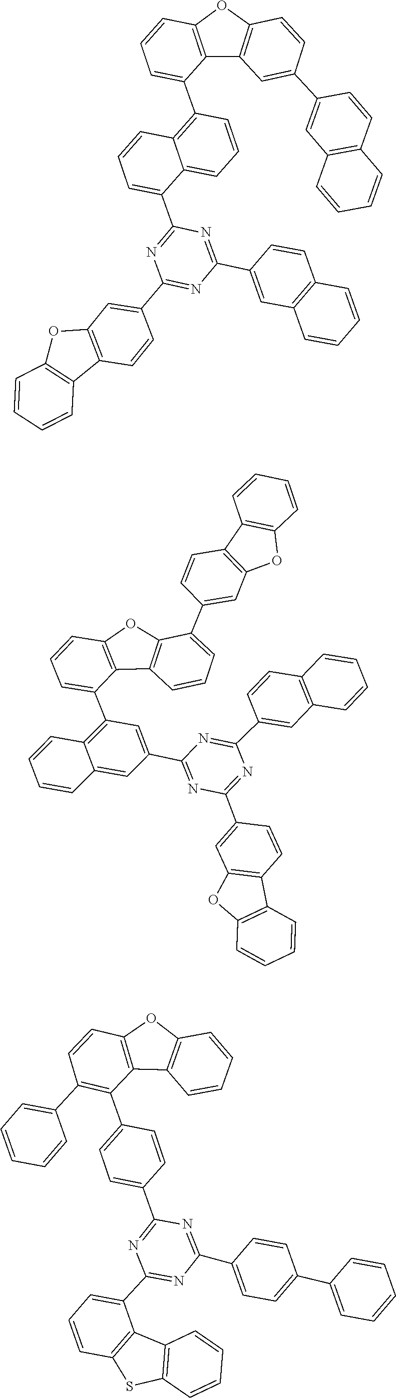 [SMILES: c1ccc2cc(-c3ccc4oc5cccc(-c6cccc7c(-c8nc(-c9ccc%10ccccc%10c9)nc(-c9ccc%10c(c9)oc9ccccc9%10)n8)cccc67)c5c4c3)ccc2c1 ; c1ccc2cc(-c3nc(-c4cc(-c5cccc6oc7c(-c8ccc9c(c8)oc8ccccc89)cccc7c56)c5ccccc5c4)nc(-c4ccc5c(c4)oc4ccccc45)n3)ccc2c1 ; c1ccc(-c2ccc(-c3nc(-c4ccc(-c5c(-c6ccccc6)ccc6oc7ccccc7c56)cc4)nc(-c4cccc5sc6ccccc6c45)n3)cc2)cc1]

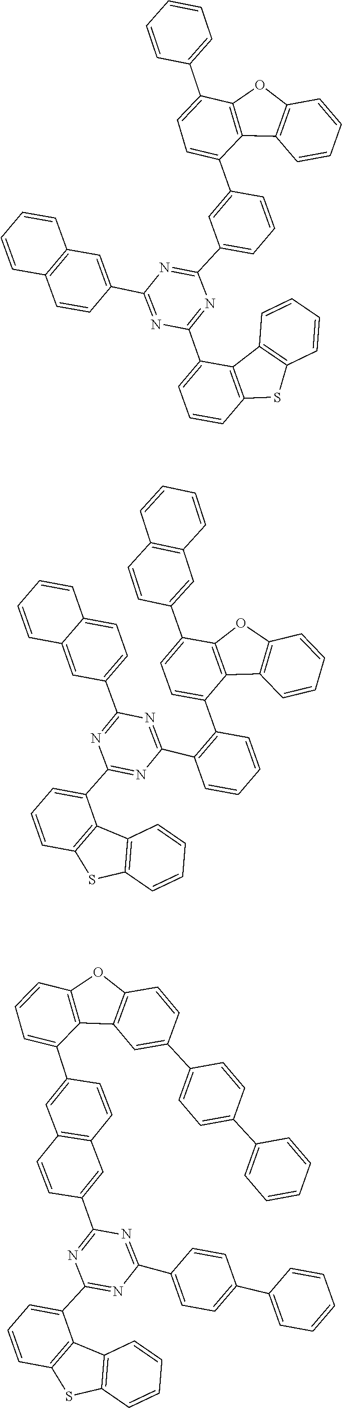 [SMILES: c1ccc(-c2ccc(-c3cccc(-c4nc(-c5ccc6ccccc6c5)nc(-c5cccc6sc7ccccc7c56)n4)c3)c3c2oc2ccccc23)cc1 ; c1ccc(-c2ccc(-c3ccc4ccccc4c3)c3oc4ccccc4c23)c(-c2nc(-c3ccc4ccccc4c3)nc(-c3cccc4sc5ccccc5c34)n2)c1 ; c1ccc(-c2ccc(-c3ccc4oc5cccc(-c6ccc7cc(-c8nc(-c9ccc(-c%10ccccc%10)cc9)nc(-c9cccc%10sc%11ccccc%11c9%10)n8)ccc7c6)c5c4c3)cc2)cc1]

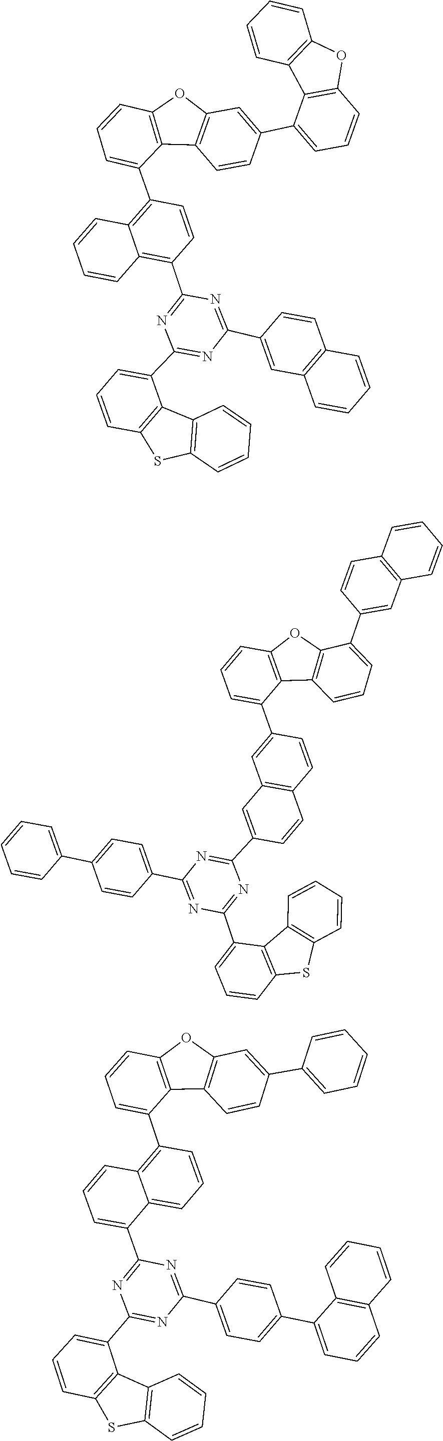 [SMILES: c1ccc2cc(-c3nc(-c4ccc(-c5cccc6oc7cc(-c8cccc9oc%10ccccc%10c89)ccc7c56)c5ccccc45)nc(-c4cccc5sc6ccccc6c45)n3)ccc2c1 ; c1ccc(-c2ccc(-c3nc(-c4ccc5ccc(-c6cccc7oc8c(-c9ccc%10ccccc%10c9)cccc8c67)cc5c4)nc(-c4cccc5sc6ccccc6c45)n3)cc2)cc1 ; c1ccc(-c2ccc3c(c2)oc2cccc(-c4cccc5c(-c6nc(-c7ccc(-c8cccc9ccccc89)cc7)nc(-c7cccc8sc9ccccc9c78)n6)cccc45)c23)cc1]

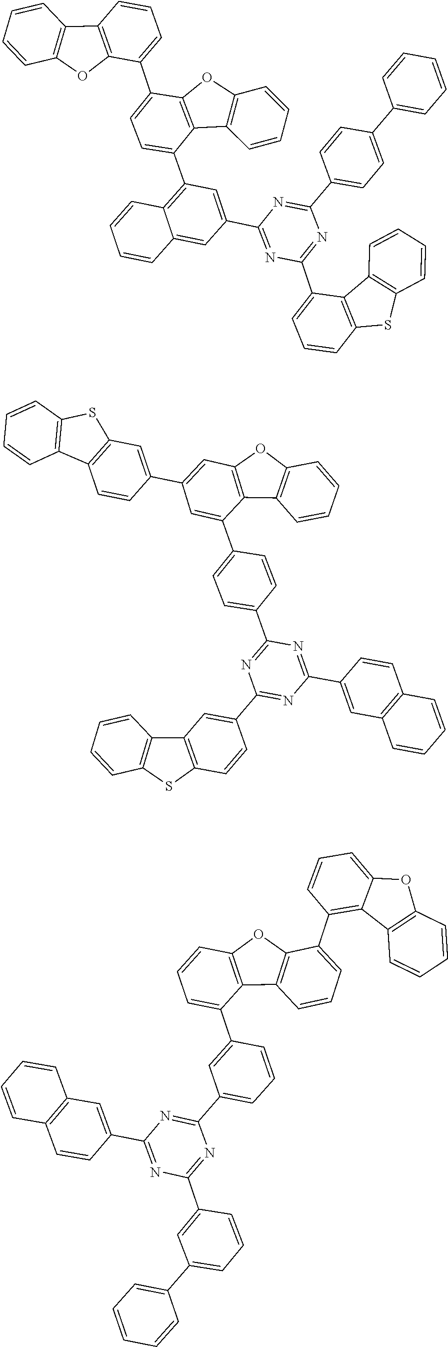 [SMILES: c1ccc(-c2ccc(-c3nc(-c4cc(-c5ccc(-c6cccc7c6oc6ccccc67)c6oc7ccccc7c56)c5ccccc5c4)nc(-c4cccc5sc6ccccc6c45)n3)cc2)cc1 ; c1ccc2cc(-c3nc(-c4ccc(-c5cc(-c6ccc7c(c6)sc6ccccc67)cc6oc7ccccc7c56)cc4)nc(-c4ccc5sc6ccccc6c5c4)n3)ccc2c1 ; c1ccc(-c2cccc(-c3nc(-c4cccc(-c5cccc6oc7c(-c8cccc9oc%10ccccc%10c89)cccc7c56)c4)nc(-c4ccc5ccccc5c4)n3)c2)cc1]

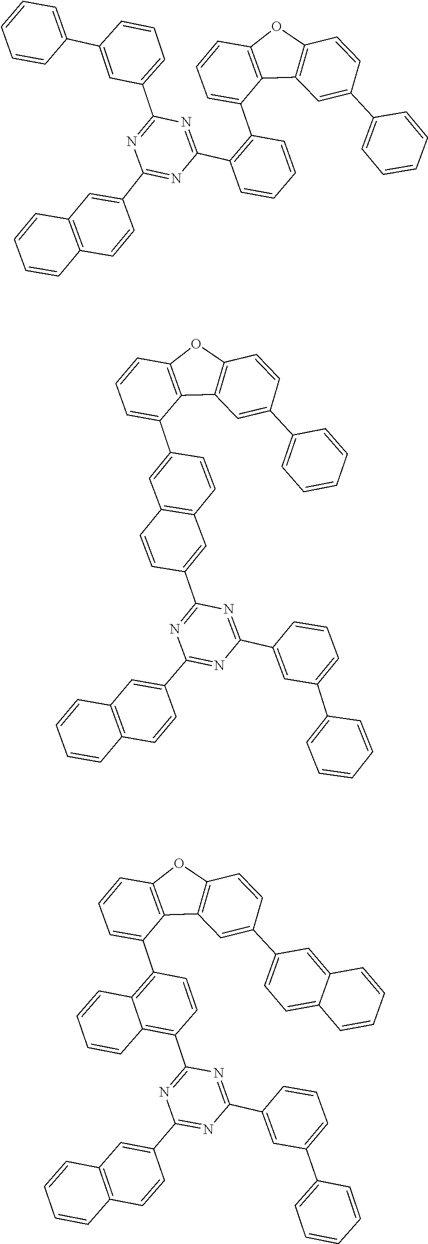 [SMILES: c1ccc(-c2cccc(-c3nc(-c4ccc5ccccc5c4)nc(-c4ccccc4-c4cccc5oc6ccc(-c7ccccc7)cc6c45)n3)c2)cc1 ; c1ccc(-c2cccc(-c3nc(-c4ccc5ccccc5c4)nc(-c4ccc5cc(-c6cccc7oc8ccc(-c9ccccc9)cc8c67)ccc5c4)n3)c2)cc1 ; c1ccc(-c2cccc(-c3nc(-c4ccc5ccccc5c4)nc(-c4ccc(-c5cccc6oc7ccc(-c8ccc9ccccc9c8)cc7c56)c5ccccc45)n3)c2)cc1]

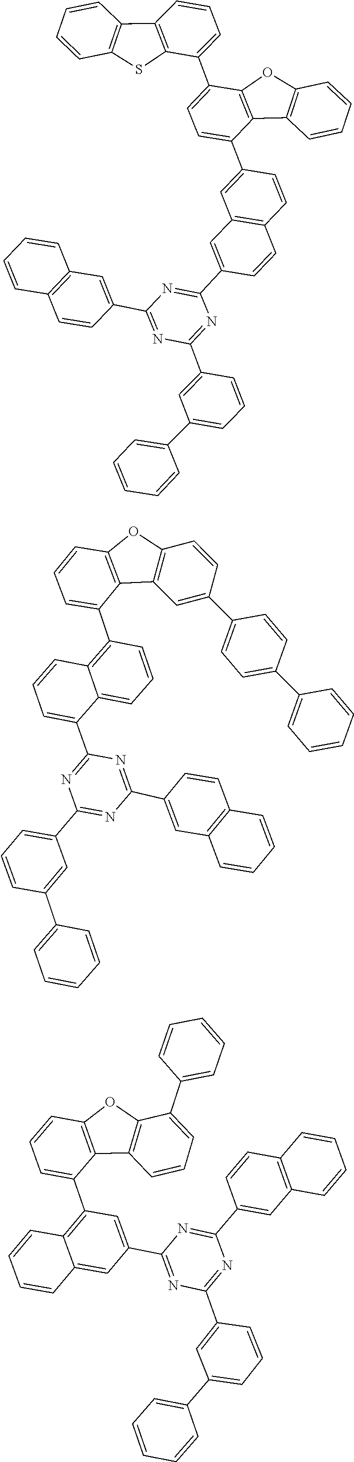

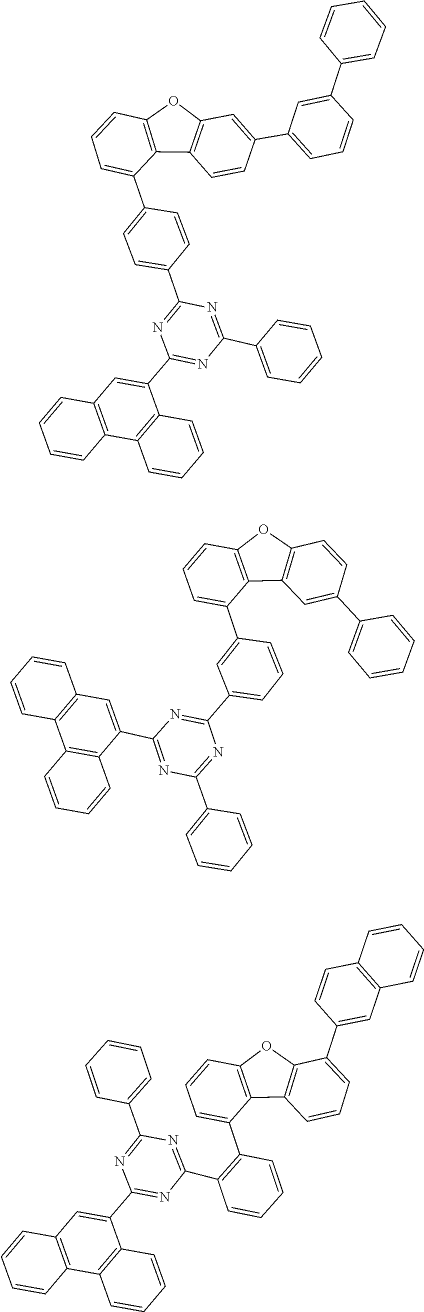 [SMILES: c1ccc(-c2cccc(-c3ccc4c(c3)oc3cccc(-c5ccc(-c6nc(-c7ccccc7)nc(-c7cc8ccccc8c8ccccc78)n6)cc5)c34)c2)cc1 ; c1ccc(-c2ccc3oc4cccc(-c5cccc(-c6nc(-c7ccccc7)nc(-c7cc8ccccc8c8ccccc78)n6)c5)c4c3c2)cc1 ; c1ccc(-c2nc(-c3ccccc3-c3cccc4oc5c(-c6ccc7ccccc7c6)cccc5c34)nc(-c3cc4ccccc4c4ccccc34)n2)cc1]

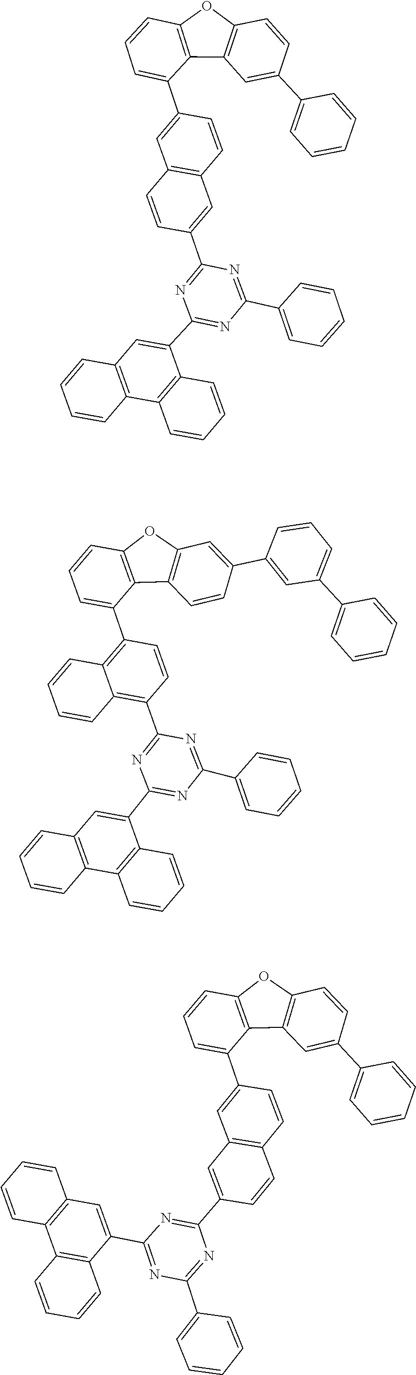 [SMILES: c1ccc(-c2ccc3oc4cccc(-c5ccc6cc(-c7nc(-c8ccccc8)nc(-c8cc9ccccc9c9ccccc89)n7)ccc6c5)c4c3c2)cc1 ; c1ccc(-c2cccc(-c3ccc4c(c3)oc3cccc(-c5ccc(-c6nc(-c7ccccc7)nc(-c7cc8ccccc8c8ccccc78)n6)c6ccccc56)c34)c2)cc1 ; c1ccc(-c2ccc3oc4cccc(-c5ccc6ccc(-c7nc(-c8ccccc8)nc(-c8cc9ccccc9c9ccccc89)n7)cc6c5)c4c3c2)cc1]

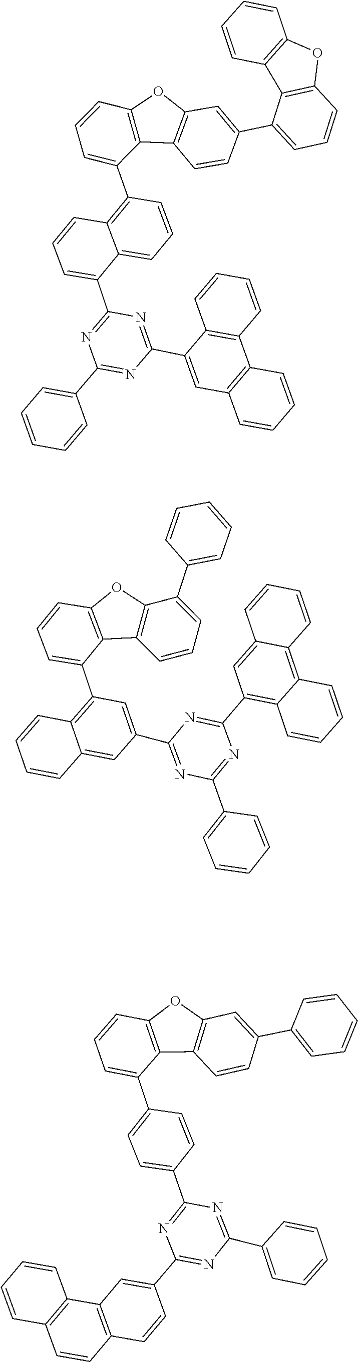 [SMILES: c1ccc(-c2nc(-c3cccc4c(-c5cccc6oc7cc(-c8cccc9oc%10ccccc%10c89)ccc7c56)cccc34)nc(-c3cc4ccccc4c4ccccc34)n2)cc1 ; c1ccc(-c2nc(-c3cc(-c4cccc5oc6c(-c7ccccc7)cccc6c45)c4ccccc4c3)nc(-c3cc4ccccc4c4ccccc34)n2)cc1 ; c1ccc(-c2ccc3c(c2)oc2cccc(-c4ccc(-c5nc(-c6ccccc6)nc(-c6ccc7ccc8ccccc8c7c6)n5)cc4)c23)cc1]

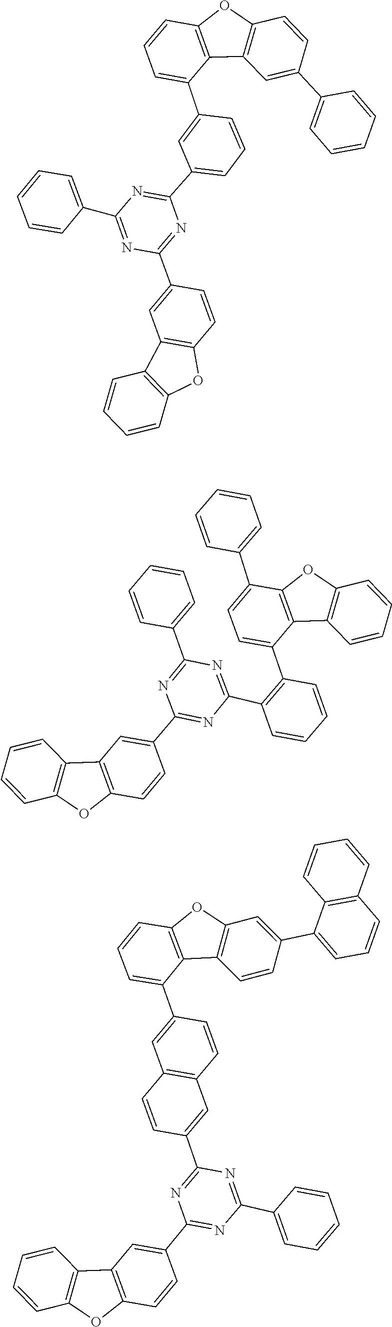 [SMILES: c1ccc(-c2ccc3oc4cccc(-c5cccc(-c6nc(-c7ccccc7)nc(-c7ccc8oc9ccccc9c8c7)n6)c5)c4c3c2)cc1 ; c1ccc(-c2nc(-c3ccc4oc5ccccc5c4c3)nc(-c3ccccc3-c3ccc(-c4ccccc4)c4oc5ccccc5c34)n2)cc1 ; c1ccc(-c2nc(-c3ccc4cc(-c5cccc6oc7cc(-c8cccc9ccccc89)ccc7c56)ccc4c3)nc(-c3ccc4oc5ccccc5c4c3)n2)cc1]

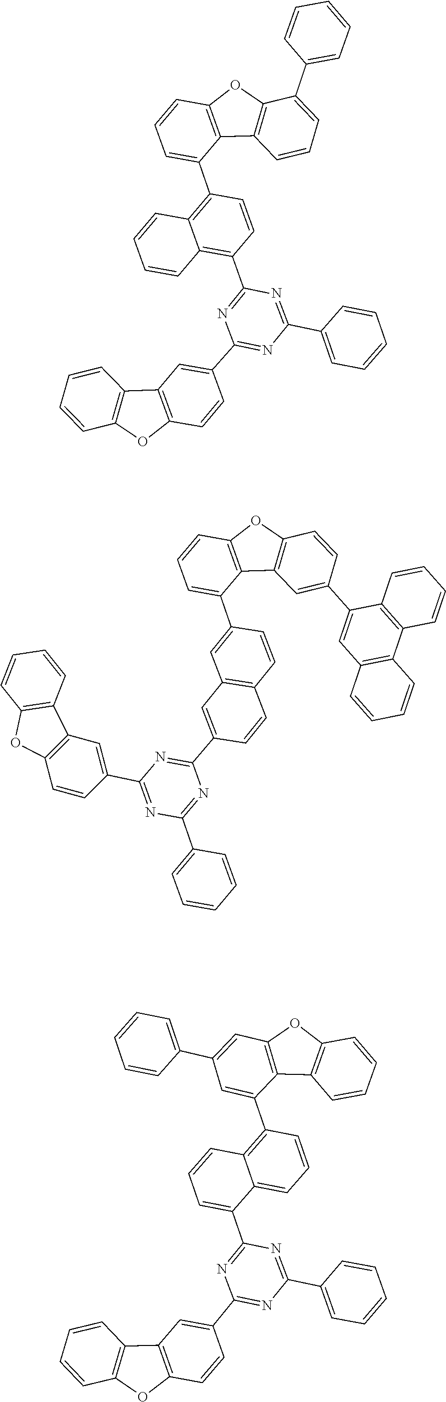 [SMILES: c1ccc(-c2nc(-c3ccc4oc5ccccc5c4c3)nc(-c3ccc(-c4cccc5oc6c(-c7ccccc7)cccc6c45)c4ccccc34)n2)cc1 ; c1ccc(-c2nc(-c3ccc4ccc(-c5cccc6oc7ccc(-c8cc9ccccc9c9ccccc89)cc7c56)cc4c3)nc(-c3ccc4oc5ccccc5c4c3)n2)cc1 ; c1ccc(-c2cc(-c3cccc4c(-c5nc(-c6ccccc6)nc(-c6ccc7oc8ccccc8c7c6)n5)cccc34)c3c(c2)oc2ccccc23)cc1]

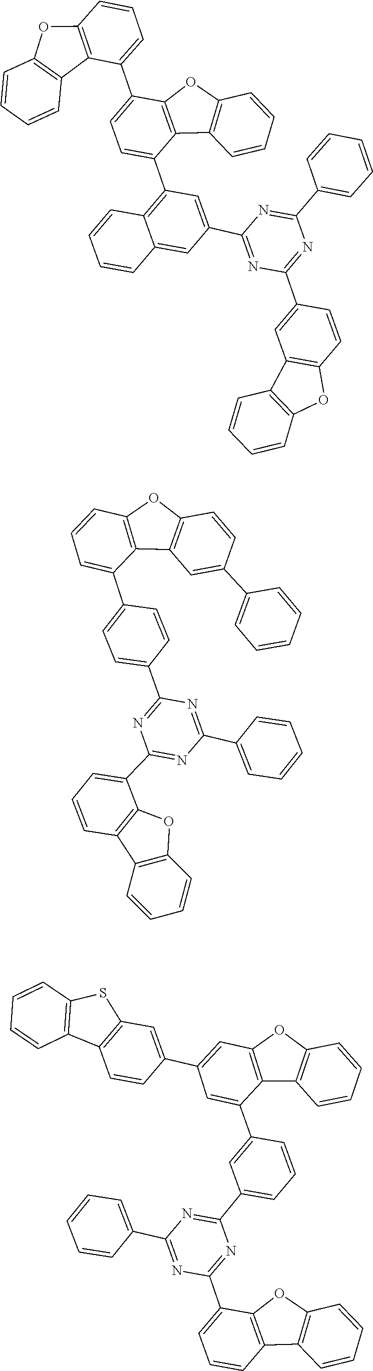 [SMILES: c1ccc(-c2nc(-c3cc(-c4ccc(-c5cccc6oc7ccccc7c56)c5oc6ccccc6c45)c4ccccc4c3)nc(-c3ccc4oc5ccccc5c4c3)n2)cc1 ; c1ccc(-c2ccc3oc4cccc(-c5ccc(-c6nc(-c7ccccc7)nc(-c7cccc8c7oc7ccccc78)n6)cc5)c4c3c2)cc1 ; c1ccc(-c2nc(-c3cccc(-c4cc(-c5ccc6c(c5)sc5ccccc56)cc5oc6ccccc6c45)c3)nc(-c3cccc4c3oc3ccccc34)n2)cc1]

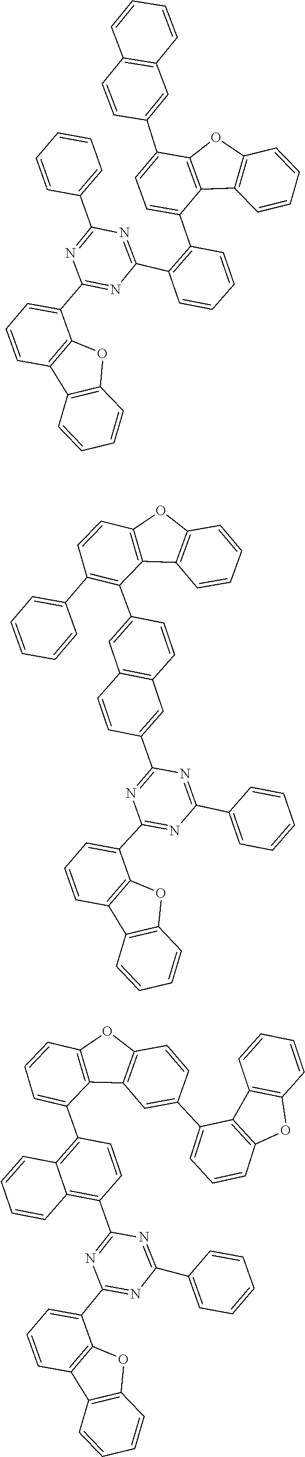 [SMILES: c1ccc(-c2nc(-c3ccccc3-c3ccc(-c4ccc5ccccc5c4)c4oc5ccccc5c34)nc(-c3cccc4c3oc3ccccc34)n2)cc1 ; c1ccc(-c2nc(-c3ccc4cc(-c5c(-c6ccccc6)ccc6oc7ccccc7c56)ccc4c3)nc(-c3cccc4c3oc3ccccc34)n2)cc1 ; c1ccc(-c2nc(-c3ccc(-c4cccc5oc6ccc(-c7cccc8oc9ccccc9c78)cc6c45)c4ccccc34)nc(-c3cccc4c3oc3ccccc34)n2)cc1]

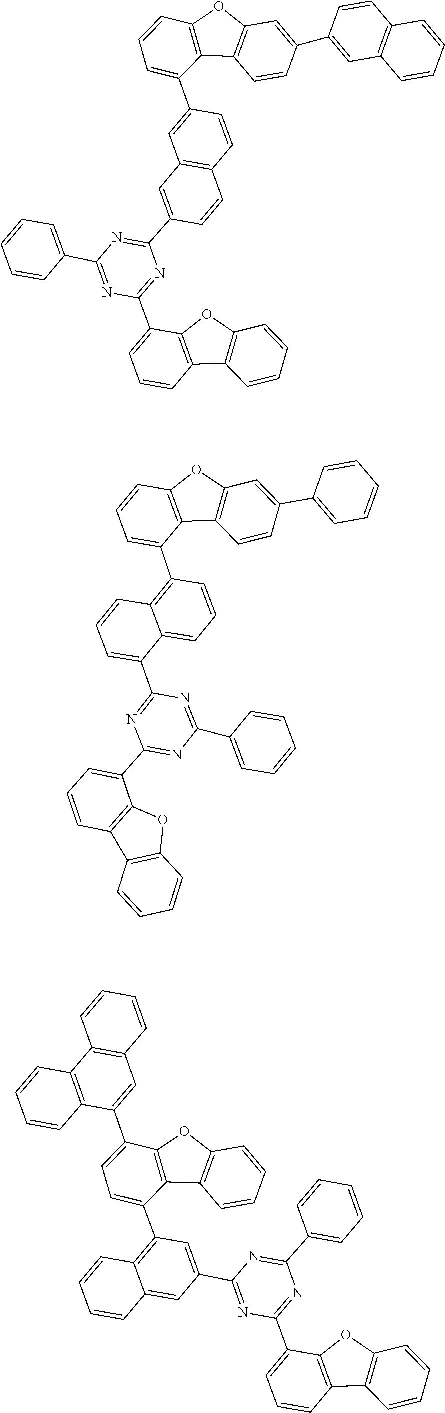 [SMILES: c1ccc(-c2nc(-c3ccc4ccc(-c5cccc6oc7cc(-c8ccc9ccccc9c8)ccc7c56)cc4c3)nc(-c3cccc4c3oc3ccccc34)n2)cc1 ; c1ccc(-c2ccc3c(c2)oc2cccc(-c4cccc5c(-c6nc(-c7ccccc7)nc(-c7cccc8c7oc7ccccc78)n6)cccc45)c23)cc1 ; c1ccc(-c2nc(-c3cc(-c4ccc(-c5cc6ccccc6c6ccccc56)c5oc6ccccc6c45)c4ccccc4c3)nc(-c3cccc4c3oc3ccccc34)n2)cc1]

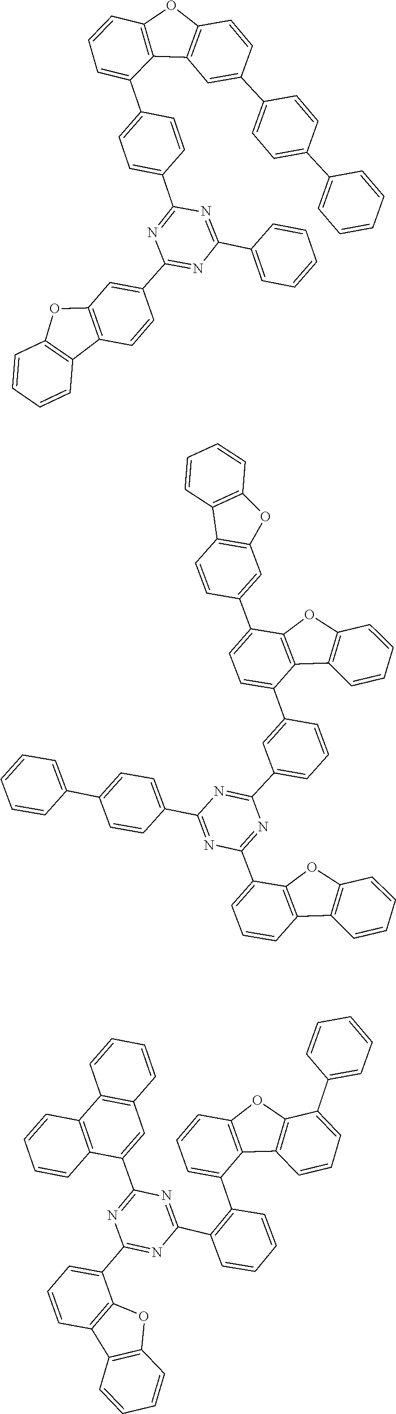 [SMILES: c1ccc(-c2ccc(-c3ccc4oc5cccc(-c6ccc(-c7nc(-c8ccccc8)nc(-c8ccc9c(c8)oc8ccccc89)n7)cc6)c5c4c3)cc2)cc1 ; c1ccc(-c2ccc(-c3nc(-c4cccc(-c5ccc(-c6ccc7c(c6)oc6ccccc67)c6oc7ccccc7c56)c4)nc(-c4cccc5c4oc4ccccc45)n3)cc2)cc1 ; c1ccc(-c2cccc3c2oc2cccc(-c4ccccc4-c4nc(-c5cc6ccccc6c6ccccc56)nc(-c5cccc6c5oc5ccccc56)n4)c23)cc1]

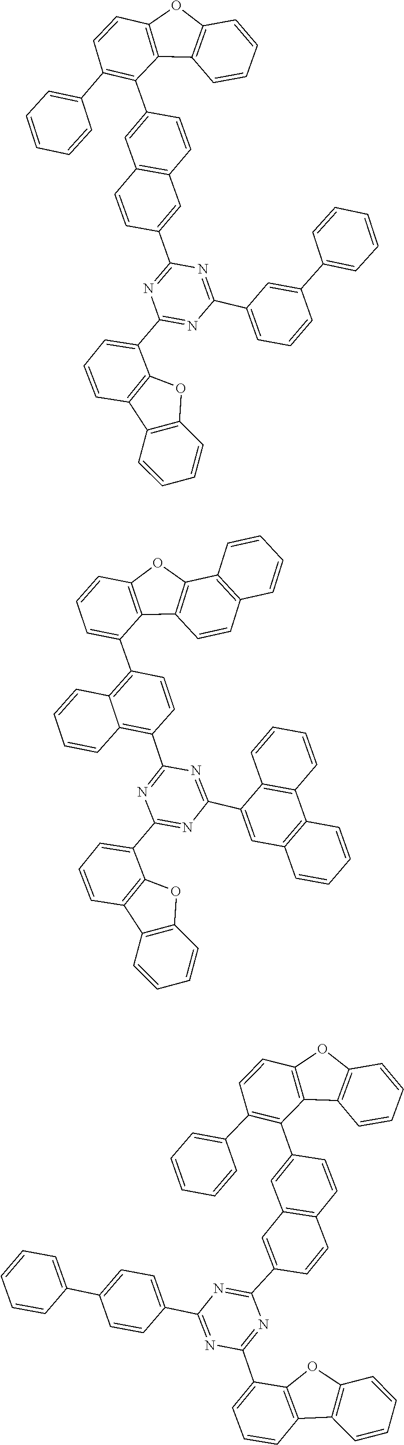 [SMILES: c1ccc(-c2cccc(-c3nc(-c4ccc5cc(-c6c(-c7ccccc7)ccc7oc8ccccc8c67)ccc5c4)nc(-c4cccc5c4oc4ccccc45)n3)c2)cc1 ; c1ccc2c(c1)cc(-c1nc(-c3ccc(-c4cccc5oc6c7ccccc7ccc6c45)c4ccccc34)nc(-c3cccc4c3oc3ccccc34)n1)c1ccccc12 ; c1ccc(-c2ccc(-c3nc(-c4ccc5ccc(-c6c(-c7ccccc7)ccc7oc8ccccc8c67)cc5c4)nc(-c4cccc5c4oc4ccccc45)n3)cc2)cc1]

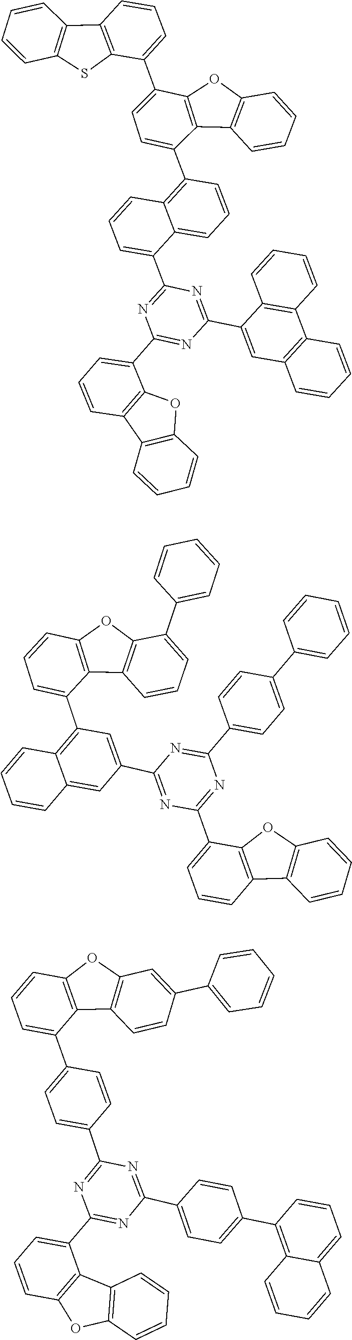 [SMILES: c1ccc2c(c1)cc(-c1nc(-c3cccc4c(-c5ccc(-c6cccc7c6sc6ccccc67)c6oc7ccccc7c56)cccc34)nc(-c3cccc4c3oc3ccccc34)n1)c1ccccc12 ; c1ccc(-c2ccc(-c3nc(-c4cc(-c5cccc6oc7c(-c8ccccc8)cccc7c56)c5ccccc5c4)nc(-c4cccc5c4oc4ccccc45)n3)cc2)cc1 ; c1ccc(-c2ccc3c(c2)oc2cccc(-c4ccc(-c5nc(-c6ccc(-c7cccc8ccccc78)cc6)nc(-c6cccc7oc8ccccc8c67)n5)cc4)c23)cc1]

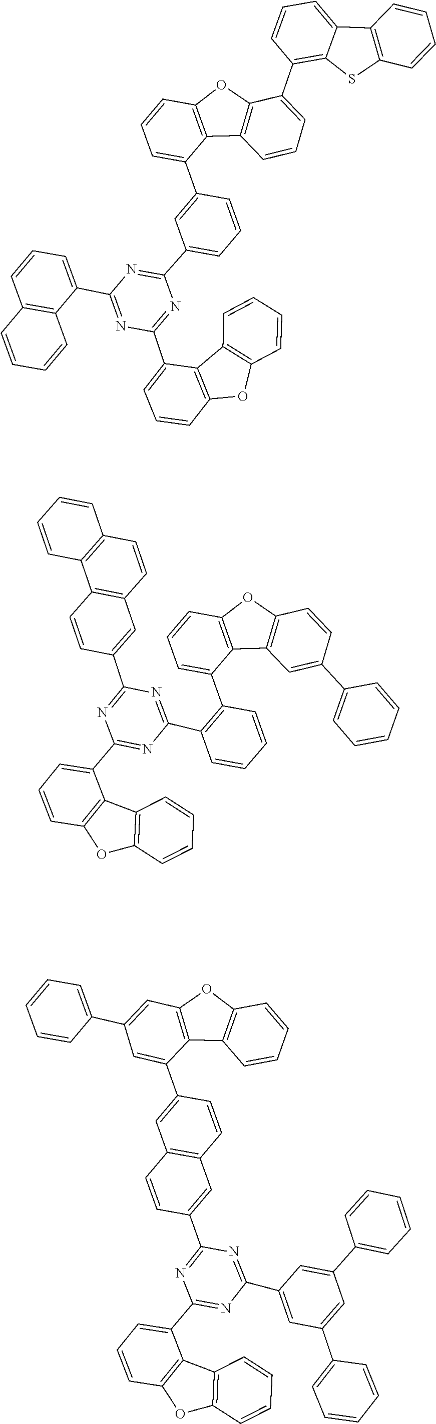 [SMILES: c1cc(-c2nc(-c3cccc4ccccc34)nc(-c3cccc4oc5ccccc5c34)n2)cc(-c2cccc3oc4c(-c5cccc6c5sc5ccccc56)cccc4c23)c1 ; c1ccc(-c2ccc3oc4cccc(-c5ccccc5-c5nc(-c6ccc7c(ccc8ccccc87)c6)nc(-c6cccc7oc8ccccc8c67)n5)c4c3c2)cc1 ; c1ccc(-c2cc(-c3ccccc3)cc(-c3nc(-c4ccc5cc(-c6cc(-c7ccccc7)cc7oc8ccccc8c67)ccc5c4)nc(-c4cccc5oc6ccccc6c45)n3)c2)cc1]

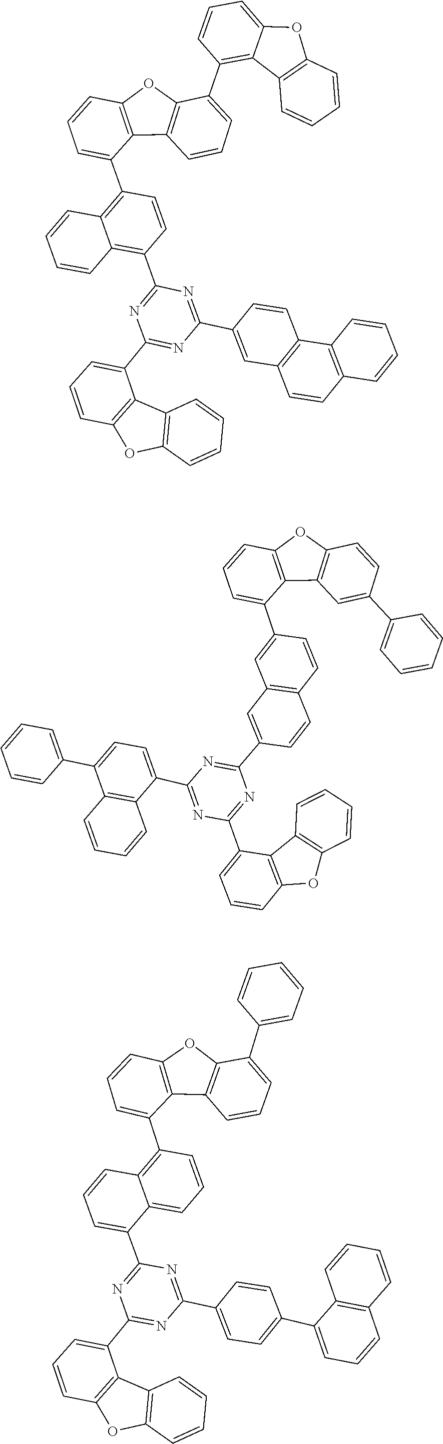 [SMILES: c1ccc2c(c1)ccc1cc(-c3nc(-c4ccc(-c5cccc6oc7c(-c8cccc9oc%10ccccc%10c89)cccc7c56)c5ccccc45)nc(-c4cccc5oc6ccccc6c45)n3)ccc12 ; c1ccc(-c2ccc3oc4cccc(-c5ccc6ccc(-c7nc(-c8ccc(-c9ccccc9)c9ccccc89)nc(-c8cccc9oc%10ccccc%10c89)n7)cc6c5)c4c3c2)cc1 ; c1ccc(-c2cccc3c2oc2cccc(-c4cccc5c(-c6nc(-c7ccc(-c8cccc9ccccc89)cc7)nc(-c7cccc8oc9ccccc9c78)n6)cccc45)c23)cc1]

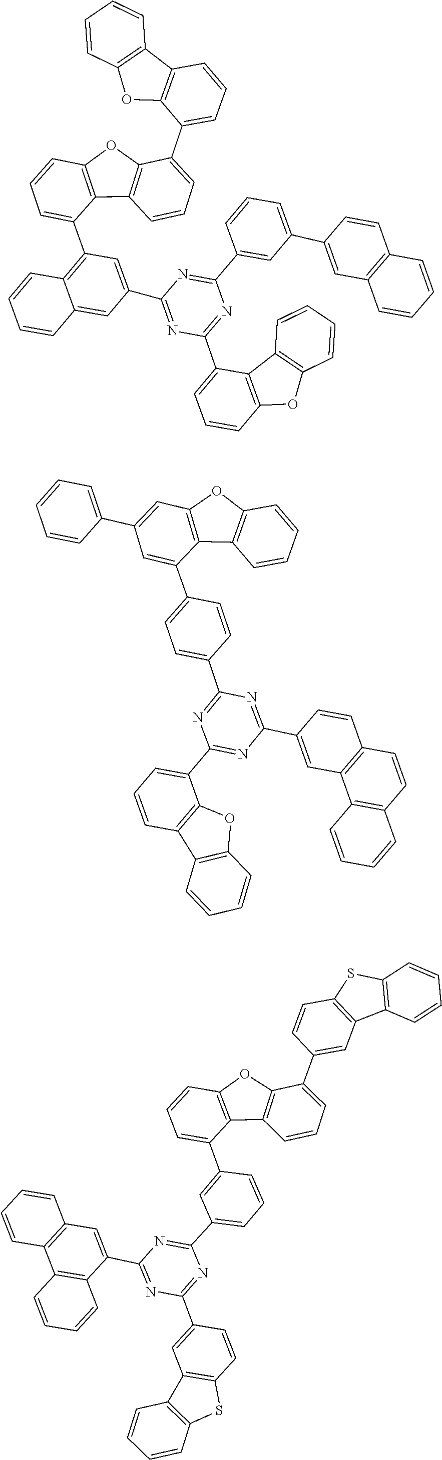 [SMILES: c1cc(-c2ccc3ccccc3c2)cc(-c2nc(-c3cc(-c4cccc5oc6c(-c7cccc8c7oc7ccccc78)cccc6c45)c4ccccc4c3)nc(-c3cccc4oc5ccccc5c34)n2)c1 ; c1ccc(-c2cc(-c3ccc(-c4nc(-c5ccc6ccc7ccccc7c6c5)nc(-c5cccc6c5oc5ccccc56)n4)cc3)c3c(c2)oc2ccccc23)cc1 ; c1cc(-c2nc(-c3ccc4sc5ccccc5c4c3)nc(-c3cc4ccccc4c4ccccc34)n2)cc(-c2cccc3oc4c(-c5ccc6sc7ccccc7c6c5)cccc4c23)c1]

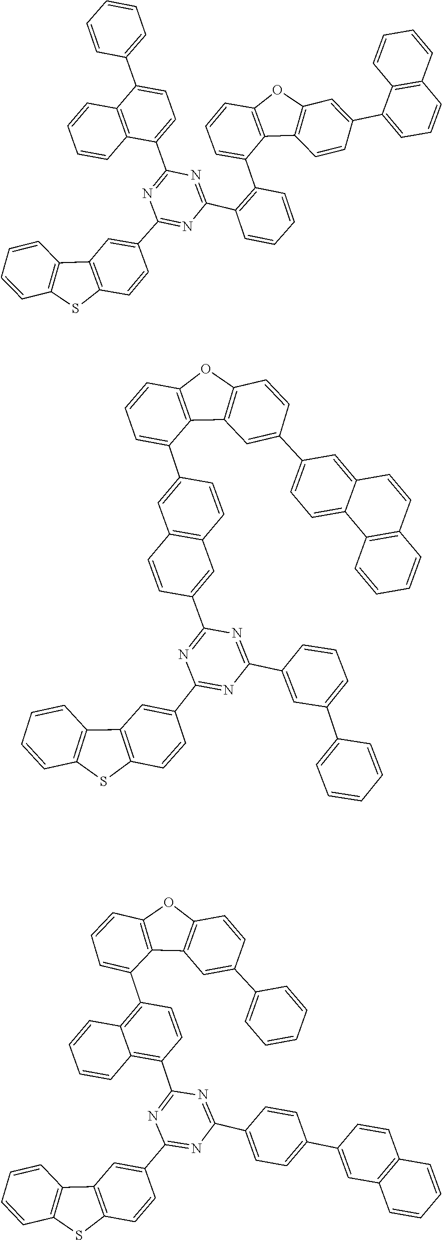 [SMILES: c1ccc(-c2ccc(-c3nc(-c4ccc5sc6ccccc6c5c4)nc(-c4ccccc4-c4cccc5oc6cc(-c7cccc8ccccc78)ccc6c45)n3)c3ccccc23)cc1 ; c1ccc(-c2cccc(-c3nc(-c4ccc5cc(-c6cccc7oc8ccc(-c9ccc%10c(ccc%11ccccc%11%10)c9)cc8c67)ccc5c4)nc(-c4ccc5sc6ccccc6c5c4)n3)c2)cc1 ; c1ccc(-c2ccc3oc4cccc(-c5ccc(-c6nc(-c7ccc(-c8ccc9ccccc9c8)cc7)nc(-c7ccc8sc9ccccc9c8c7)n6)c6ccccc56)c4c3c2)cc1]

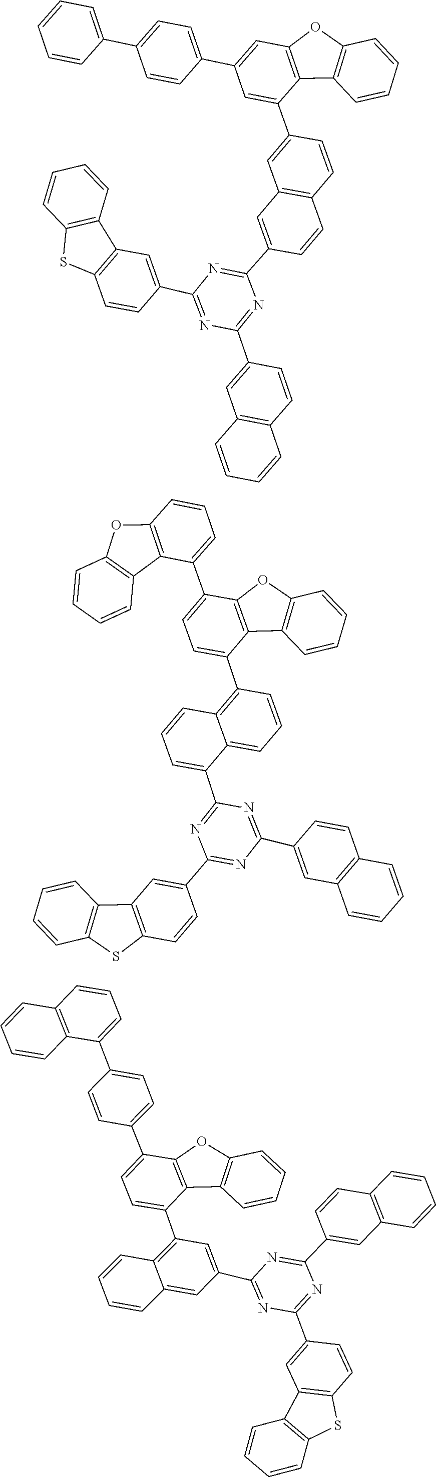

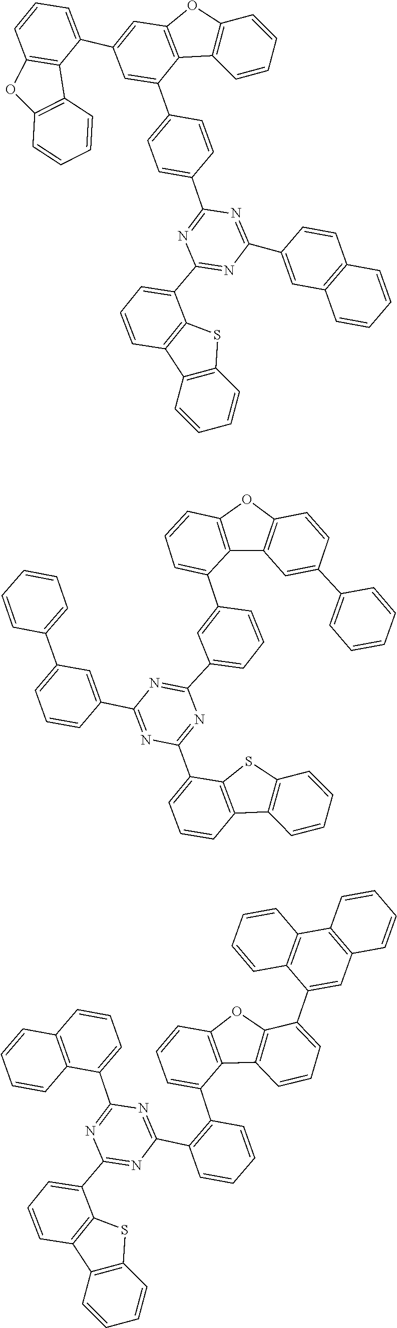 [SMILES: c1ccc2cc(-c3nc(-c4ccc(-c5cc(-c6cccc7oc8ccccc8c67)cc6oc7ccccc7c56)cc4)nc(-c4cccc5c4sc4ccccc45)n3)ccc2c1 ; c1ccc(-c2cccc(-c3nc(-c4cccc(-c5cccc6oc7ccc(-c8ccccc8)cc7c56)c4)nc(-c4cccc5c4sc4ccccc45)n3)c2)cc1 ; c1ccc(-c2cccc3oc4c(-c5cc6ccccc6c6ccccc56)cccc4c23)c(-c2nc(-c3cccc4ccccc34)nc(-c3cccc4c3sc3ccccc34)n2)c1]

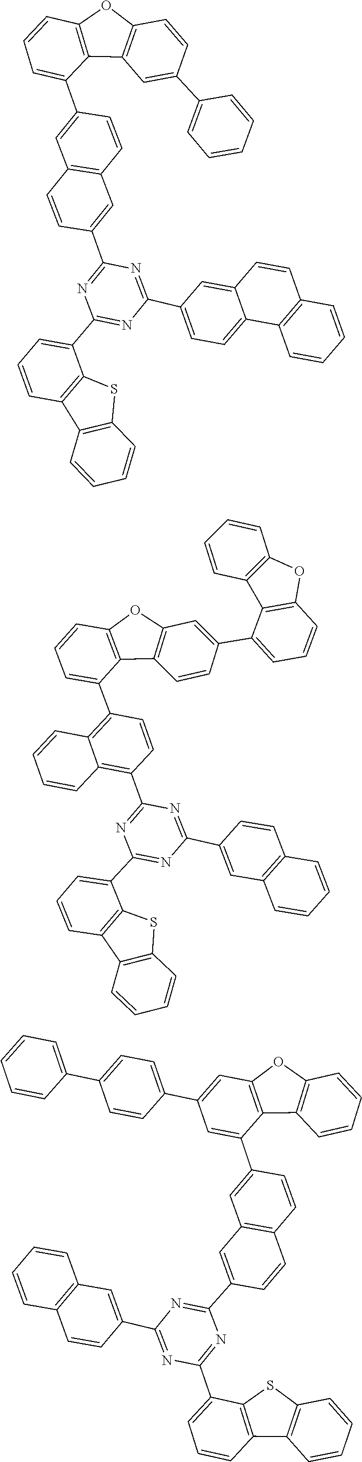

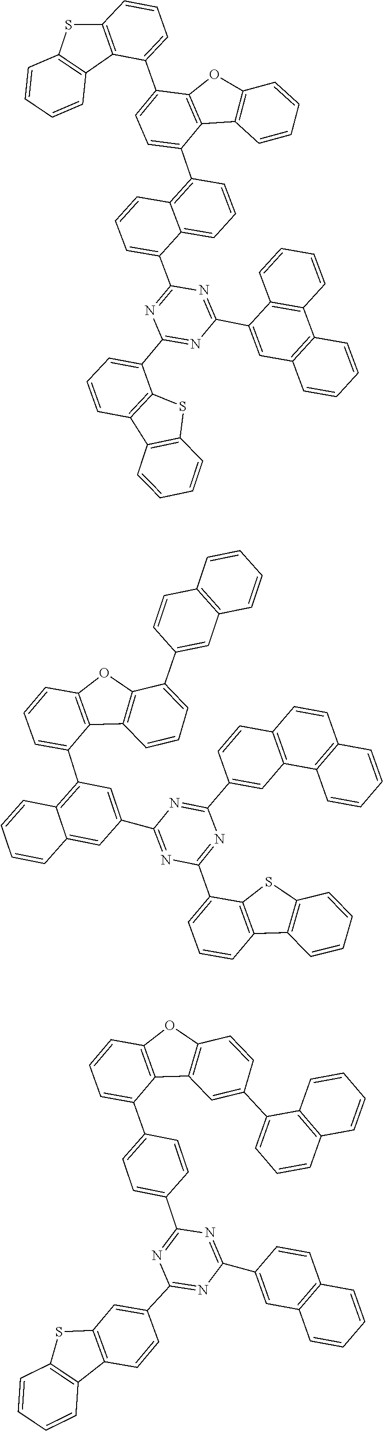 [SMILES: c1ccc2c(c1)cc(-c1nc(-c3cccc4c(-c5ccc(-c6cccc7sc8ccccc8c67)c6oc7ccccc7c56)cccc34)nc(-c3cccc4c3sc3ccccc34)n1)c1ccccc12 ; c1ccc2cc(-c3cccc4c3oc3cccc(-c5cc(-c6nc(-c7ccc8ccc9ccccc9c8c7)nc(-c7cccc8c7sc7ccccc78)n6)cc6ccccc56)c34)ccc2c1 ; c1ccc2cc(-c3nc(-c4ccc(-c5cccc6oc7ccc(-c8cccc9ccccc89)cc7c56)cc4)nc(-c4ccc5c(c4)sc4ccccc45)n3)ccc2c1]

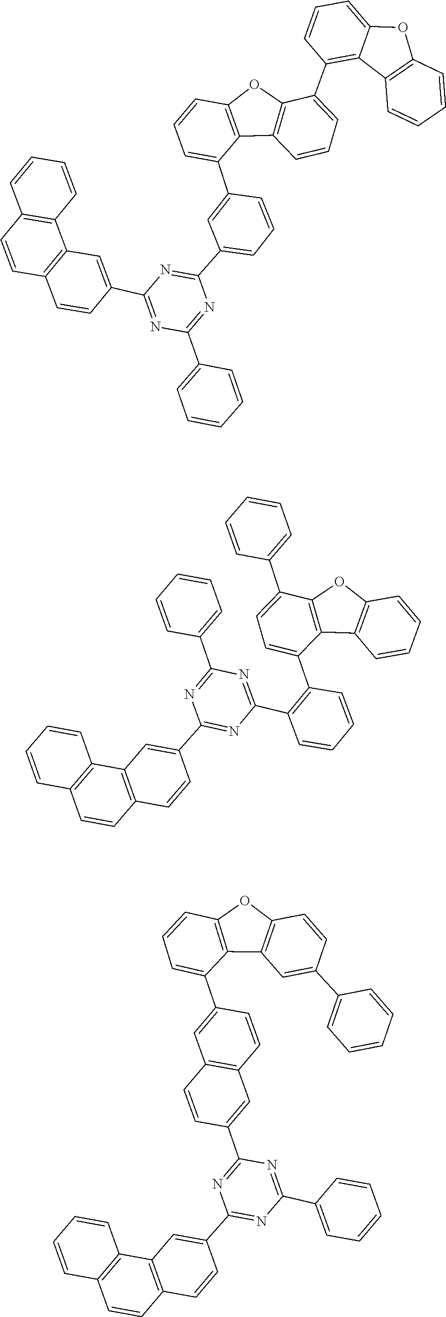 [SMILES: c1ccc(-c2nc(-c3cccc(-c4cccc5oc6c(-c7cccc8oc9ccccc9c78)cccc6c45)c3)nc(-c3ccc4ccc5ccccc5c4c3)n2)cc1 ; c1ccc(-c2nc(-c3ccc4ccc5ccccc5c4c3)nc(-c3ccccc3-c3ccc(-c4ccccc4)c4oc5ccccc5c34)n2)cc1 ; c1ccc(-c2ccc3oc4cccc(-c5ccc6cc(-c7nc(-c8ccccc8)nc(-c8ccc9ccc%10ccccc%10c9c8)n7)ccc6c5)c4c3c2)cc1]

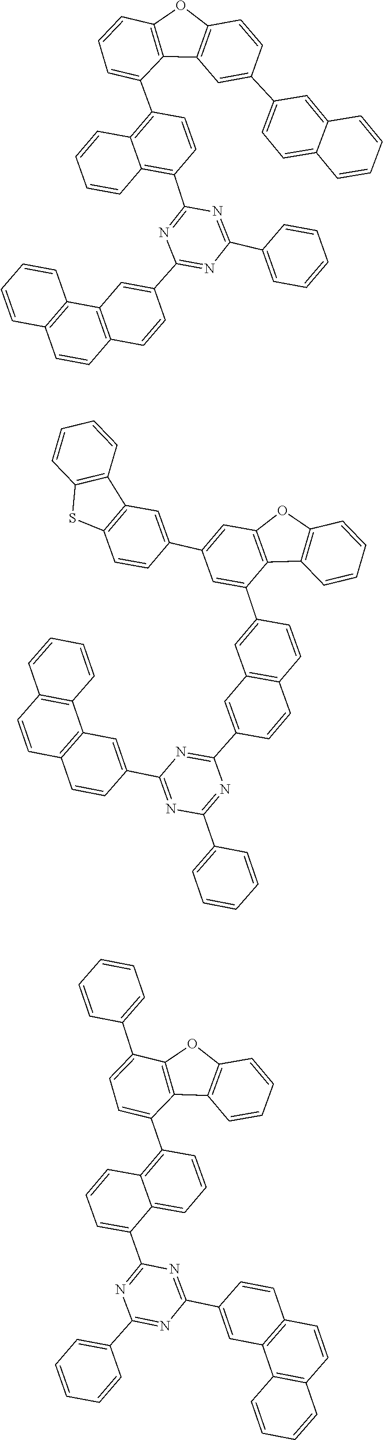 [SMILES: c1ccc(-c2nc(-c3ccc4ccc5ccccc5c4c3)nc(-c3ccc(-c4cccc5oc6ccc(-c7ccc8ccccc8c7)cc6c45)c4ccccc34)n2)cc1 ; c1ccc(-c2nc(-c3ccc4ccc(-c5cc(-c6ccc7sc8ccccc8c7c6)cc6oc7ccccc7c56)cc4c3)nc(-c3ccc4ccc5ccccc5c4c3)n2)cc1 ; c1ccc(-c2nc(-c3ccc4ccc5ccccc5c4c3)nc(-c3cccc4c(-c5ccc(-c6ccccc6)c6oc7ccccc7c56)cccc34)n2)cc1]

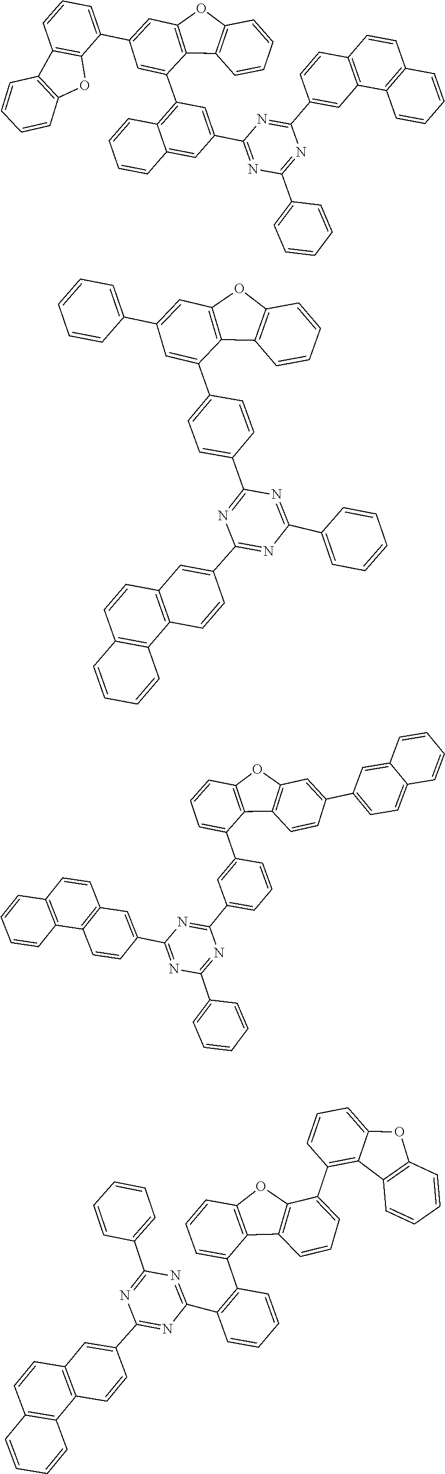 [SMILES: c1ccc(-c2nc(-c3cc(-c4cc(-c5cccc6c5oc5ccccc56)cc5oc6ccccc6c45)c4ccccc4c3)nc(-c3ccc4ccc5ccccc5c4c3)n2)cc1 ; c1ccc(-c2cc(-c3ccc(-c4nc(-c5ccccc5)nc(-c5ccc6c(ccc7ccccc76)c5)n4)cc3)c3c(c2)oc2ccccc23)cc1 ; c1ccc(-c2nc(-c3cccc(-c4cccc5oc6cc(-c7ccc8ccccc8c7)ccc6c45)c3)nc(-c3ccc4c(ccc5ccccc54)c3)n2)cc1 ; c1ccc(-c2nc(-c3ccc4c(ccc5ccccc54)c3)nc(-c3ccccc3-c3cccc4oc5c(-c6cccc7oc8ccccc8c67)cccc5c34)n2)cc1]

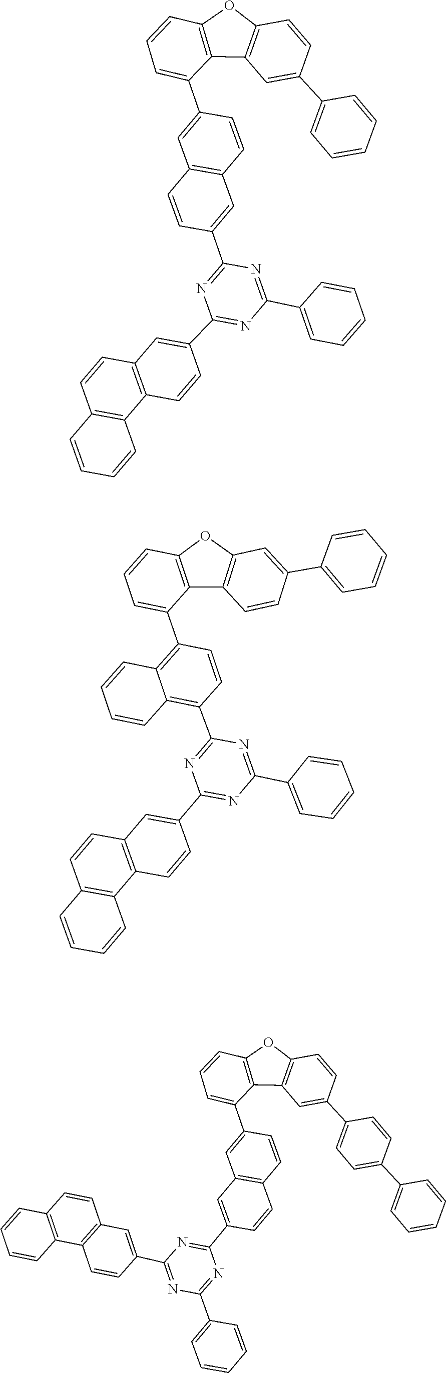 [SMILES: c1ccc(-c2ccc3oc4cccc(-c5ccc6cc(-c7nc(-c8ccccc8)nc(-c8ccc9c(ccc%10ccccc%109)c8)n7)ccc6c5)c4c3c2)cc1 ; c1ccc(-c2ccc3c(c2)oc2cccc(-c4ccc(-c5nc(-c6ccccc6)nc(-c6ccc7c(ccc8ccccc87)c6)n5)c5ccccc45)c23)cc1 ; c1ccc(-c2ccc(-c3ccc4oc5cccc(-c6ccc7ccc(-c8nc(-c9ccccc9)nc(-c9ccc%10c(ccc%11ccccc%11%10)c9)n8)cc7c6)c5c4c3)cc2)cc1]

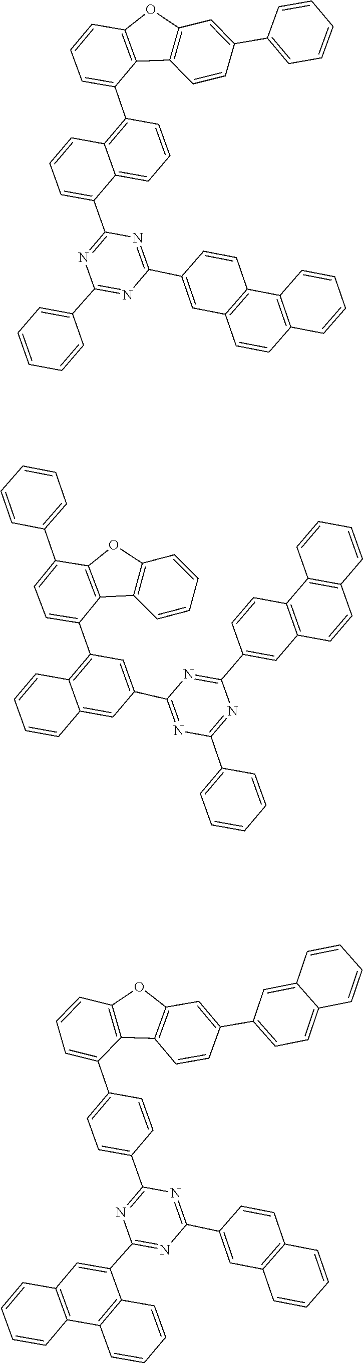 [SMILES: c1ccc(-c2ccc3c(c2)oc2cccc(-c4cccc5c(-c6nc(-c7ccccc7)nc(-c7ccc8c(ccc9ccccc98)c7)n6)cccc45)c23)cc1 ; c1ccc(-c2nc(-c3cc(-c4ccc(-c5ccccc5)c5oc6ccccc6c45)c4ccccc4c3)nc(-c3ccc4c(ccc5ccccc54)c3)n2)cc1 ; c1ccc2cc(-c3ccc4c(c3)oc3cccc(-c5ccc(-c6nc(-c7ccc8ccccc8c7)nc(-c7cc8ccccc8c8ccccc78)n6)cc5)c34)ccc2c1]

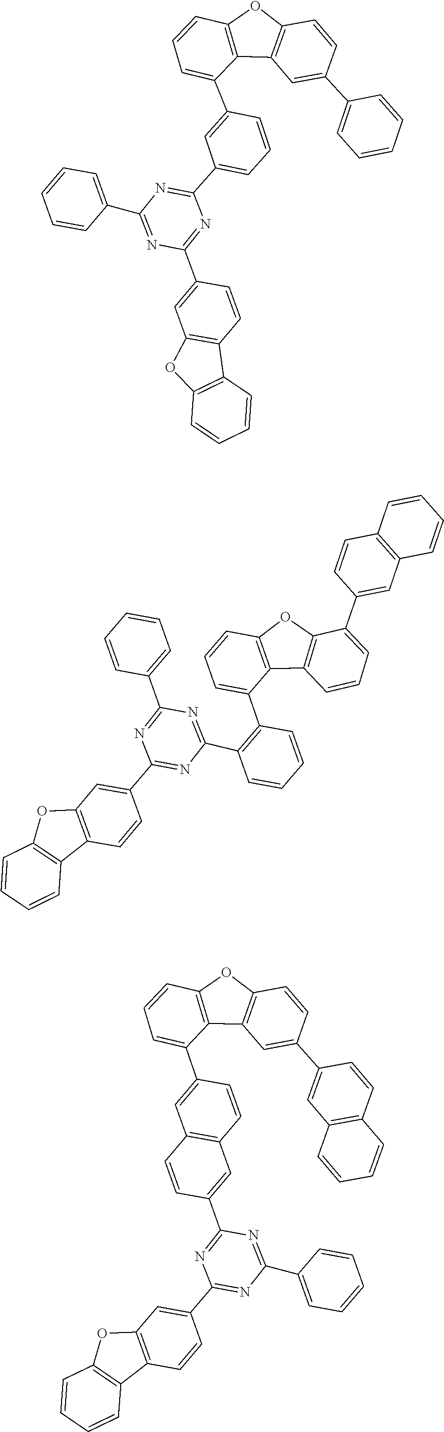 [SMILES: c1ccc(-c2ccc3oc4cccc(-c5cccc(-c6nc(-c7ccccc7)nc(-c7ccc8c(c7)oc7ccccc78)n6)c5)c4c3c2)cc1 ; c1ccc(-c2nc(-c3ccc4c(c3)oc3ccccc34)nc(-c3ccccc3-c3cccc4oc5c(-c6ccc7ccccc7c6)cccc5c34)n2)cc1 ; c1ccc(-c2nc(-c3ccc4cc(-c5cccc6oc7ccc(-c8ccc9ccccc9c8)cc7c56)ccc4c3)nc(-c3ccc4c(c3)oc3ccccc34)n2)cc1]

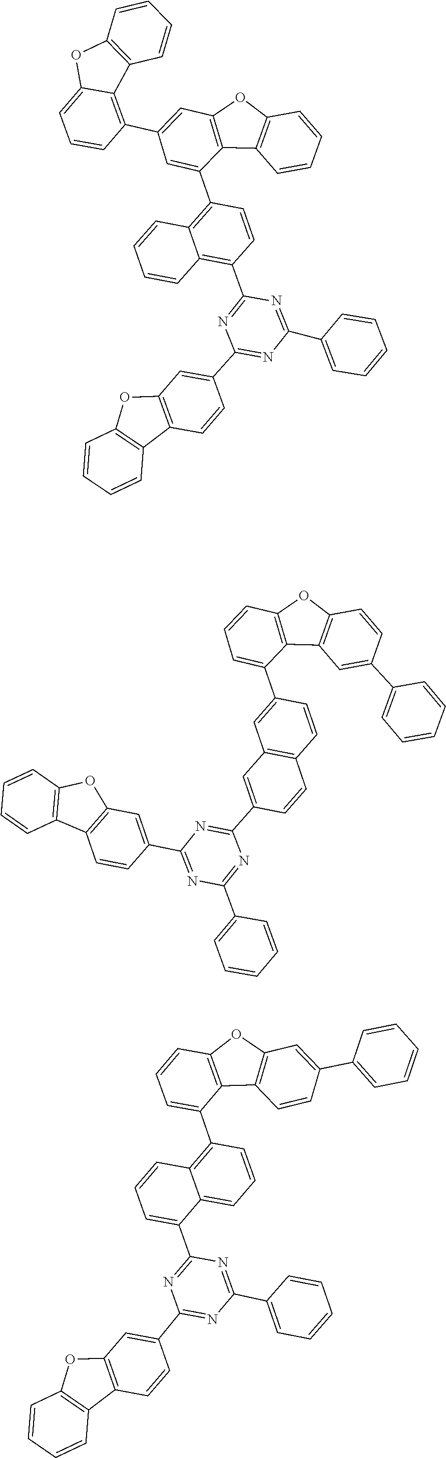 [SMILES: c1ccc(-c2nc(-c3ccc4c(c3)oc3ccccc34)nc(-c3ccc(-c4cc(-c5cccc6oc7ccccc7c56)cc5oc6ccccc6c45)c4ccccc34)n2)cc1 ; c1ccc(-c2ccc3oc4cccc(-c5ccc6ccc(-c7nc(-c8ccccc8)nc(-c8ccc9c(c8)oc8ccccc89)n7)cc6c5)c4c3c2)cc1 ; c1ccc(-c2ccc3c(c2)oc2cccc(-c4cccc5c(-c6nc(-c7ccccc7)nc(-c7ccc8c(c7)oc7ccccc78)n6)cccc45)c23)cc1]

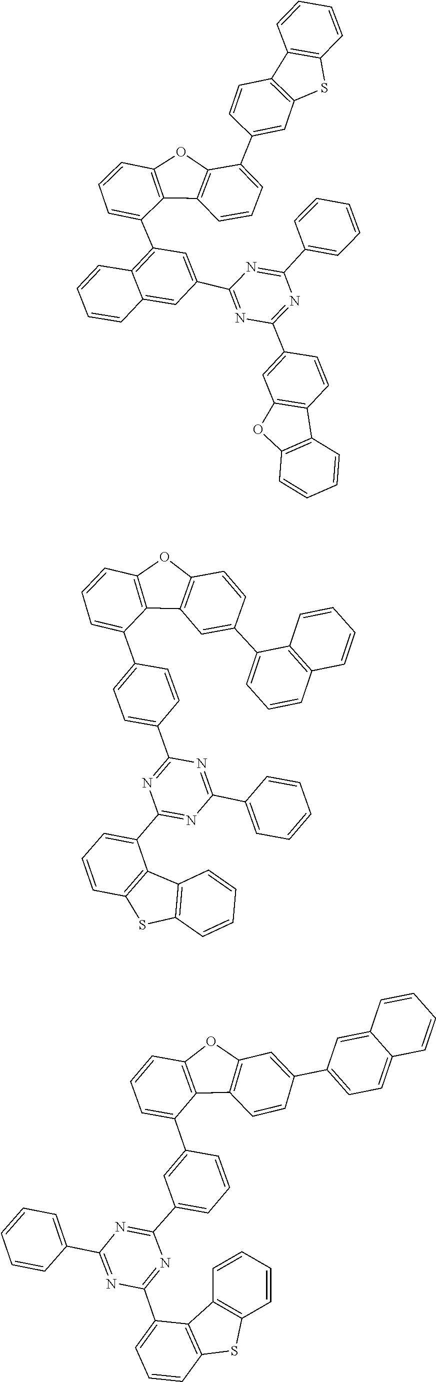 [SMILES: c1ccc(-c2nc(-c3cc(-c4cccc5oc6c(-c7ccc8c(c7)sc7ccccc78)cccc6c45)c4ccccc4c3)nc(-c3ccc4c(c3)oc3ccccc34)n2)cc1 ; c1ccc(-c2nc(-c3ccc(-c4cccc5oc6ccc(-c7cccc8ccccc78)cc6c45)cc3)nc(-c3cccc4sc5ccccc5c34)n2)cc1 ; c1ccc(-c2nc(-c3cccc(-c4cccc5oc6cc(-c7ccc8ccccc8c7)ccc6c45)c3)nc(-c3cccc4sc5ccccc5c34)n2)cc1]

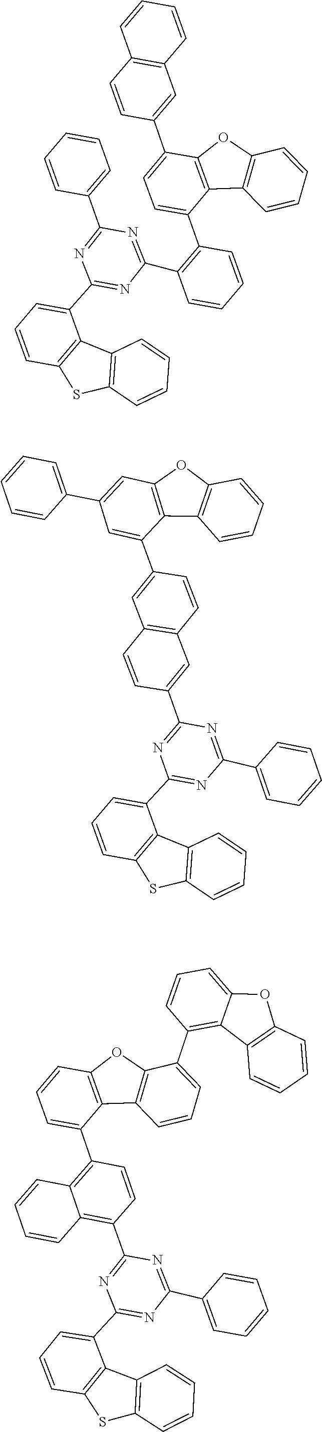 [SMILES: c1ccc(-c2nc(-c3ccccc3-c3ccc(-c4ccc5ccccc5c4)c4oc5ccccc5c34)nc(-c3cccc4sc5ccccc5c34)n2)cc1 ; c1ccc(-c2cc(-c3ccc4cc(-c5nc(-c6ccccc6)nc(-c6cccc7sc8ccccc8c67)n5)ccc4c3)c3c(c2)oc2ccccc23)cc1 ; c1ccc(-c2nc(-c3ccc(-c4cccc5oc6c(-c7cccc8oc9ccccc9c78)cccc6c45)c4ccccc34)nc(-c3cccc4sc5ccccc5c34)n2)cc1]

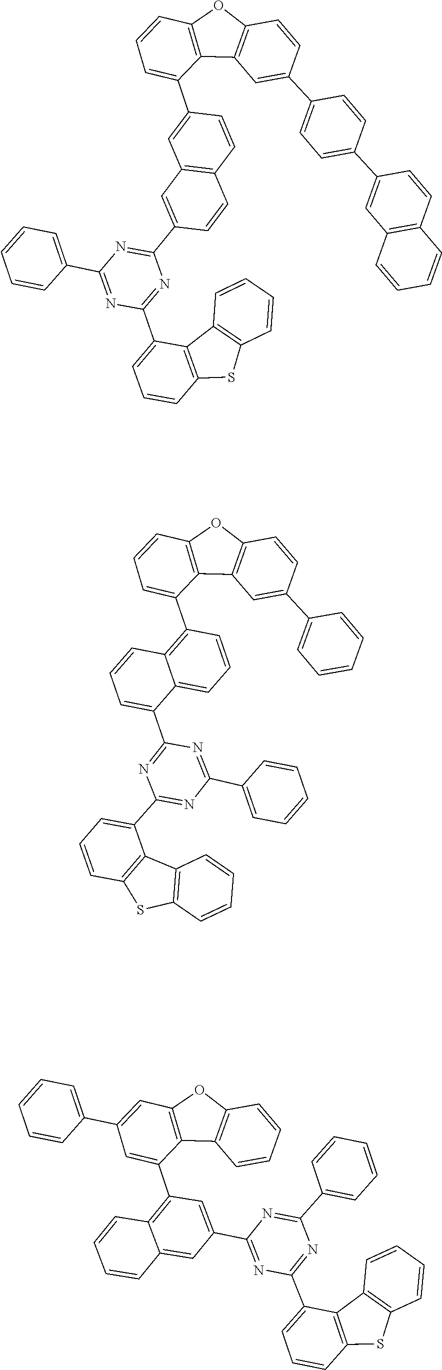 [SMILES: c1ccc(-c2nc(-c3ccc4ccc(-c5cccc6oc7ccc(-c8ccc(-c9ccc%10ccccc%10c9)cc8)cc7c56)cc4c3)nc(-c3cccc4sc5ccccc5c34)n2)cc1 ; c1ccc(-c2ccc3oc4cccc(-c5cccc6c(-c7nc(-c8ccccc8)nc(-c8cccc9sc%10ccccc%10c89)n7)cccc56)c4c3c2)cc1 ; c1ccc(-c2cc(-c3cc(-c4nc(-c5ccccc5)nc(-c5cccc6sc7ccccc7c56)n4)cc4ccccc34)c3c(c2)oc2ccccc23)cc1]

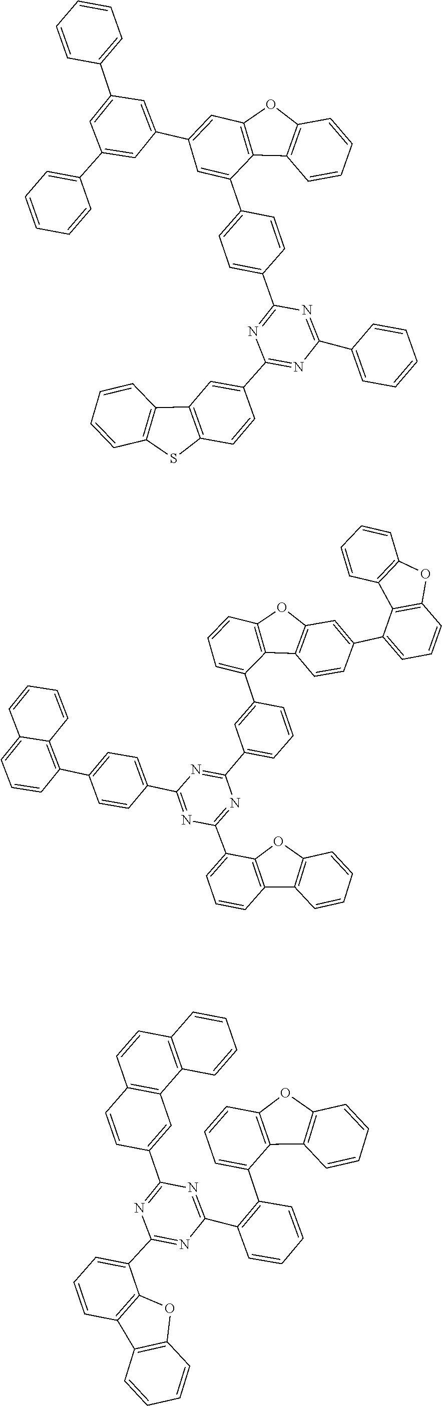 [SMILES: c1ccc(-c2cc(-c3ccccc3)cc(-c3cc(-c4ccc(-c5nc(-c6ccccc6)nc(-c6ccc7sc8ccccc8c7c6)n5)cc4)c4c(c3)oc3ccccc34)c2)cc1 ; c1cc(-c2nc(-c3ccc(-c4cccc5ccccc45)cc3)nc(-c3cccc4c3oc3ccccc34)n2)cc(-c2cccc3oc4cc(-c5cccc6oc7ccccc7c56)ccc4c23)c1 ; c1ccc(-c2cccc3oc4ccccc4c23)c(-c2nc(-c3ccc4ccc5ccccc5c4c3)nc(-c3cccc4c3oc3ccccc34)n2)c1]

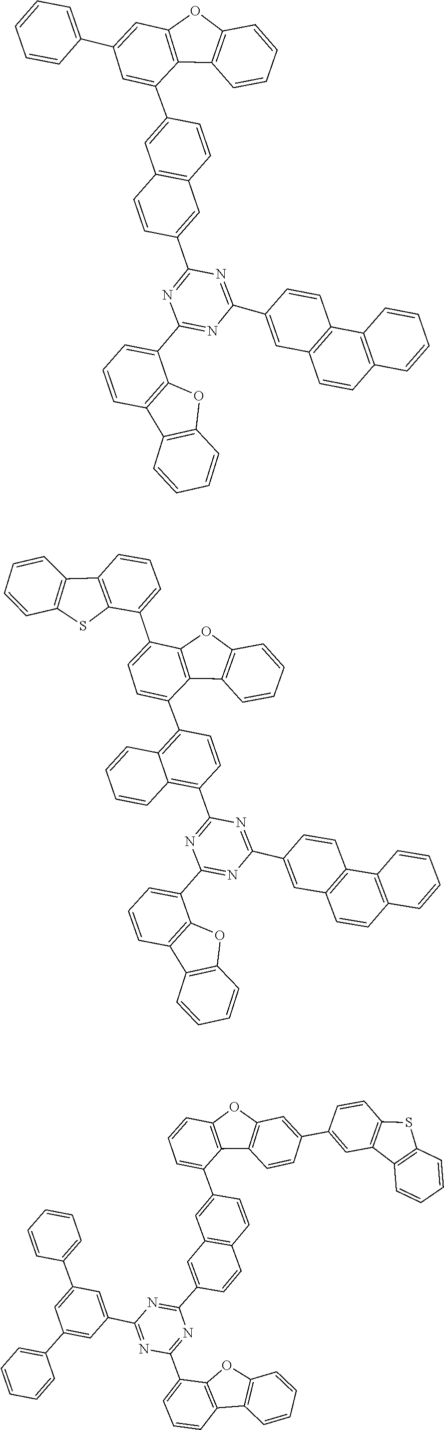 [SMILES: c1ccc(-c2cc(-c3ccc4cc(-c5nc(-c6ccc7c(ccc8ccccc87)c6)nc(-c6cccc7c6oc6ccccc67)n5)ccc4c3)c3c(c2)oc2ccccc23)cc1 ; c1ccc2c(c1)ccc1cc(-c3nc(-c4ccc(-c5ccc(-c6cccc7c6sc6ccccc67)c6oc7ccccc7c56)c5ccccc45)nc(-c4cccc5c4oc4ccccc45)n3)ccc12 ; c1ccc(-c2cc(-c3ccccc3)cc(-c3nc(-c4ccc5ccc(-c6cccc7oc8cc(-c9ccc%10sc%11ccccc%11c%10c9)ccc8c67)cc5c4)nc(-c4cccc5c4oc4ccccc45)n3)c2)cc1]

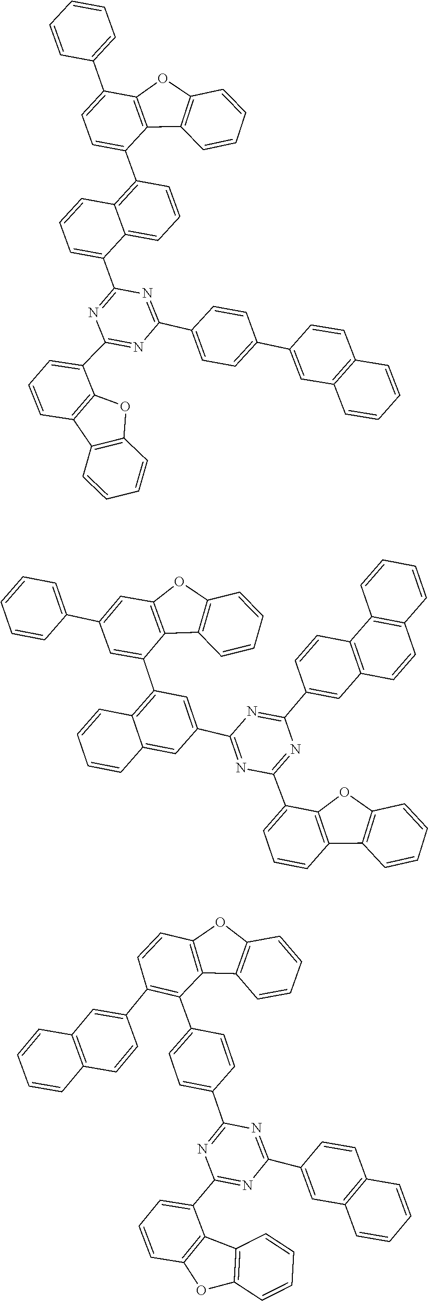 [SMILES: c1ccc(-c2ccc(-c3cccc4c(-c5nc(-c6ccc(-c7ccc8ccccc8c7)cc6)nc(-c6cccc7c6oc6ccccc67)n5)cccc34)c3c2oc2ccccc23)cc1 ; c1ccc(-c2cc(-c3cc(-c4nc(-c5ccc6c(ccc7ccccc76)c5)nc(-c5cccc6c5oc5ccccc56)n4)cc4ccccc34)c3c(c2)oc2ccccc23)cc1 ; c1ccc2cc(-c3nc(-c4ccc(-c5c(-c6ccc7ccccc7c6)ccc6oc7ccccc7c56)cc4)nc(-c4cccc5oc6ccccc6c45)n3)ccc2c1]

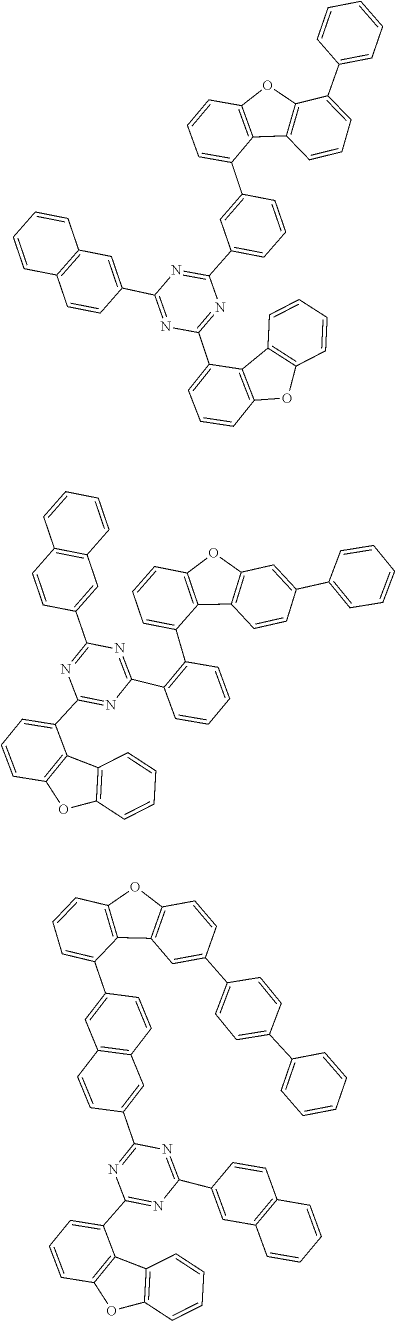 [SMILES: c1ccc(-c2cccc3c2oc2cccc(-c4cccc(-c5nc(-c6ccc7ccccc7c6)nc(-c6cccc7oc8ccccc8c67)n5)c4)c23)cc1 ; c1ccc(-c2ccc3c(c2)oc2cccc(-c4ccccc4-c4nc(-c5ccc6ccccc6c5)nc(-c5cccc6oc7ccccc7c56)n4)c23)cc1 ; c1ccc(-c2ccc(-c3ccc4oc5cccc(-c6ccc7cc(-c8nc(-c9ccc%10ccccc%10c9)nc(-c9cccc%10oc%11ccccc%11c9%10)n8)ccc7c6)c5c4c3)cc2)cc1]

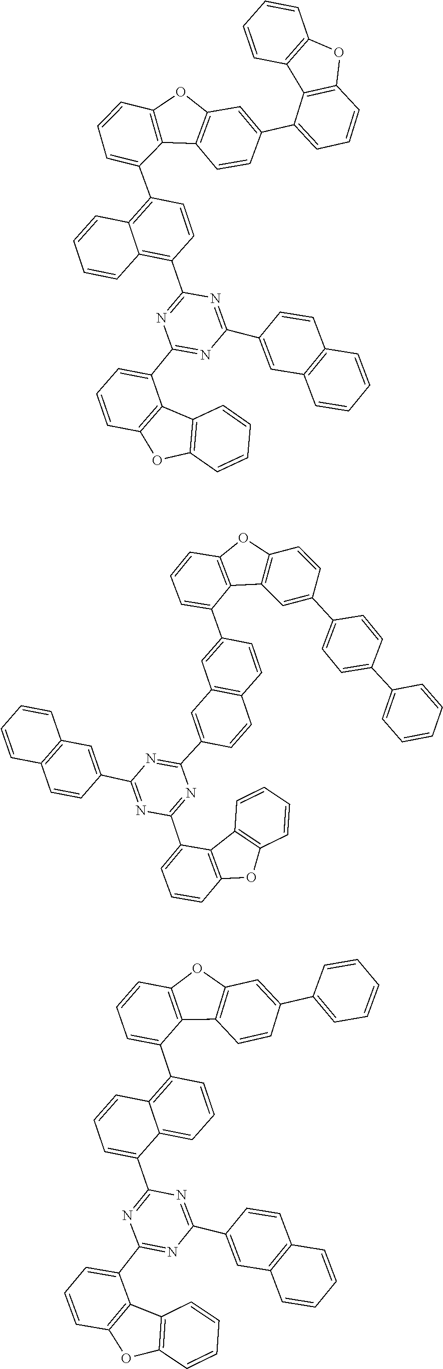 [SMILES: c1ccc2cc(-c3nc(-c4ccc(-c5cccc6oc7cc(-c8cccc9oc%10ccccc%10c89)ccc7c56)c5ccccc45)nc(-c4cccc5oc6ccccc6c45)n3)ccc2c1 ; c1ccc(-c2ccc(-c3ccc4oc5cccc(-c6ccc7ccc(-c8nc(-c9ccc%10ccccc%10c9)nc(-c9cccc%10oc%11ccccc%11c9%10)n8)cc7c6)c5c4c3)cc2)cc1 ; c1ccc(-c2ccc3c(c2)oc2cccc(-c4cccc5c(-c6nc(-c7ccc8ccccc8c7)nc(-c7cccc8oc9ccccc9c78)n6)cccc45)c23)cc1]

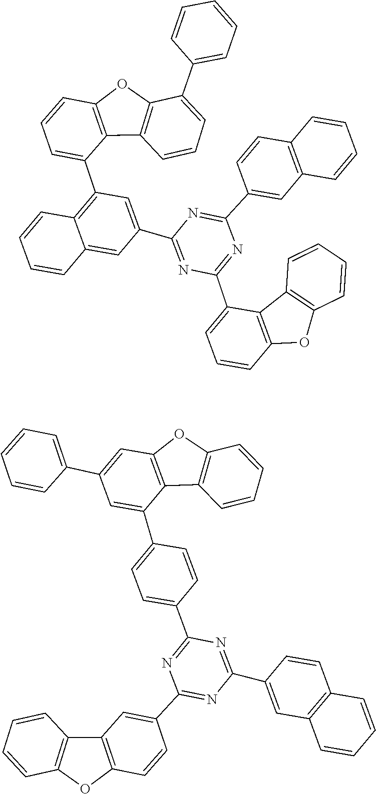 [SMILES: c1ccc(-c2cccc3c2oc2cccc(-c4cc(-c5nc(-c6ccc7ccccc7c6)nc(-c6cccc7oc8ccccc8c67)n5)cc5ccccc45)c23)cc1 ; c1ccc(-c2cc(-c3ccc(-c4nc(-c5ccc6ccccc6c5)nc(-c5ccc6oc7ccccc7c6c5)n4)cc3)c3c(c2)oc2ccccc23)cc1]

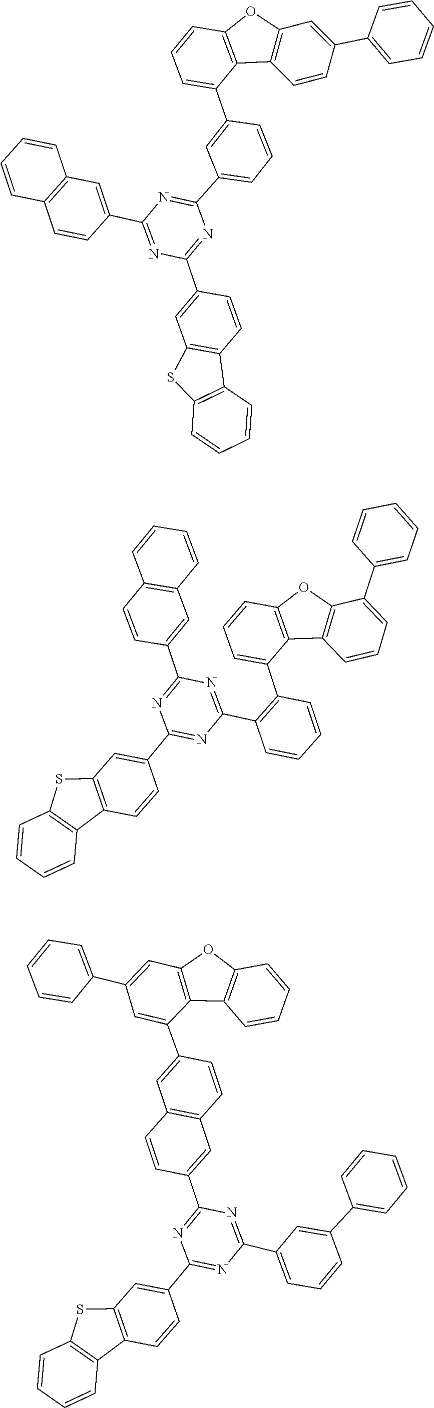 [SMILES: c1ccc(-c2ccc3c(c2)oc2cccc(-c4cccc(-c5nc(-c6ccc7ccccc7c6)nc(-c6ccc7c(c6)sc6ccccc67)n5)c4)c23)cc1 ; c1ccc(-c2cccc3c2oc2cccc(-c4ccccc4-c4nc(-c5ccc6ccccc6c5)nc(-c5ccc6c(c5)sc5ccccc56)n4)c23)cc1 ; c1ccc(-c2cccc(-c3nc(-c4ccc5cc(-c6cc(-c7ccccc7)cc7oc8ccccc8c67)ccc5c4)nc(-c4ccc5c(c4)sc4ccccc45)n3)c2)cc1]

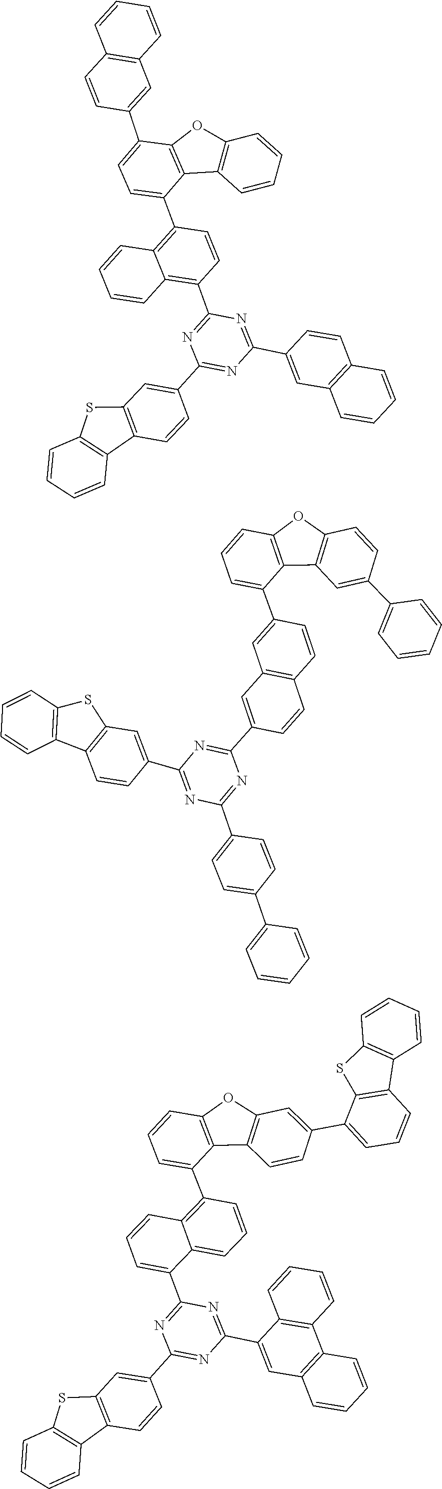 [SMILES: c1ccc2cc(-c3nc(-c4ccc5c(c4)sc4ccccc45)nc(-c4ccc(-c5ccc(-c6ccc7ccccc7c6)c6oc7ccccc7c56)c5ccccc45)n3)ccc2c1 ; c1ccc(-c2ccc(-c3nc(-c4ccc5ccc(-c6cccc7oc8ccc(-c9ccccc9)cc8c67)cc5c4)nc(-c4ccc5c(c4)sc4ccccc45)n3)cc2)cc1 ; c1ccc2c(c1)cc(-c1nc(-c3ccc4c(c3)sc3ccccc34)nc(-c3cccc4c(-c5cccc6oc7cc(-c8cccc9c8sc8ccccc89)ccc7c56)cccc34)n1)c1ccccc12]

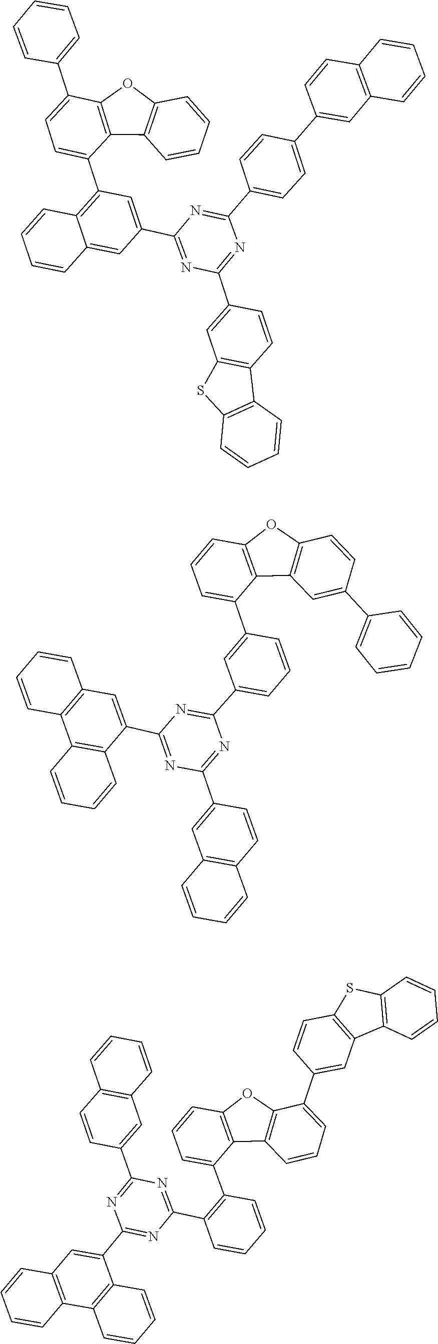 [SMILES: c1ccc(-c2ccc(-c3cc(-c4nc(-c5ccc(-c6ccc7ccccc7c6)cc5)nc(-c5ccc6c(c5)sc5ccccc56)n4)cc4ccccc34)c3c2oc2ccccc23)cc1 ; c1ccc(-c2ccc3oc4cccc(-c5cccc(-c6nc(-c7ccc8ccccc8c7)nc(-c7cc8ccccc8c8ccccc78)n6)c5)c4c3c2)cc1 ; c1ccc(-c2cccc3oc4c(-c5ccc6sc7ccccc7c6c5)cccc4c23)c(-c2nc(-c3ccc4ccccc4c3)nc(-c3cc4ccccc4c4ccccc34)n2)c1]

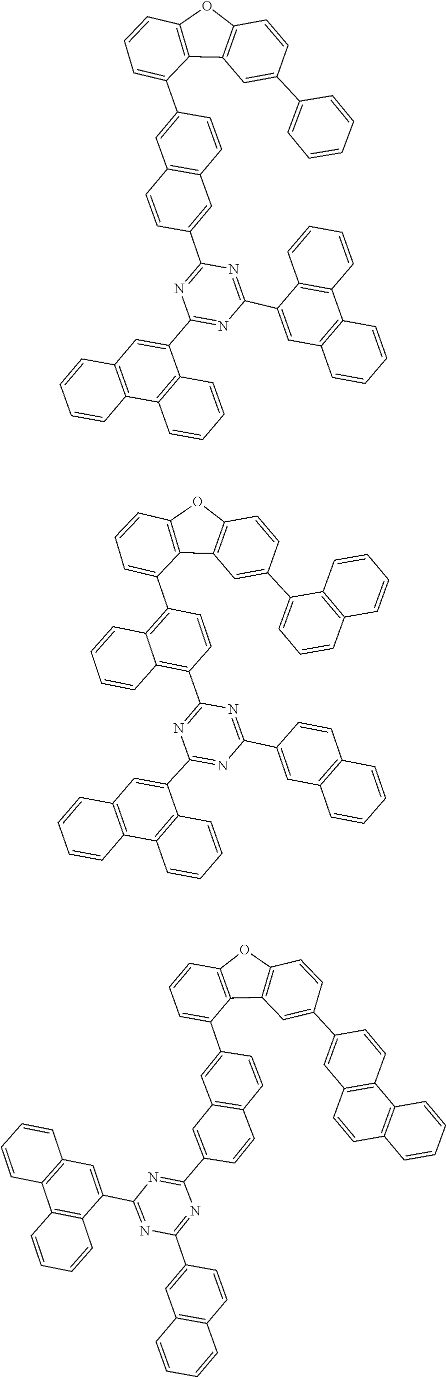 [SMILES: c1ccc(-c2ccc3oc4cccc(-c5ccc6cc(-c7nc(-c8cc9ccccc9c9ccccc89)nc(-c8cc9ccccc9c9ccccc89)n7)ccc6c5)c4c3c2)cc1 ; c1ccc2cc(-c3nc(-c4ccc(-c5cccc6oc7ccc(-c8cccc9ccccc89)cc7c56)c5ccccc45)nc(-c4cc5ccccc5c5ccccc45)n3)ccc2c1 ; c1ccc2cc(-c3nc(-c4ccc5ccc(-c6cccc7oc8ccc(-c9ccc%10c(ccc%11ccccc%11%10)c9)cc8c67)cc5c4)nc(-c4cc5ccccc5c5ccccc45)n3)ccc2c1]

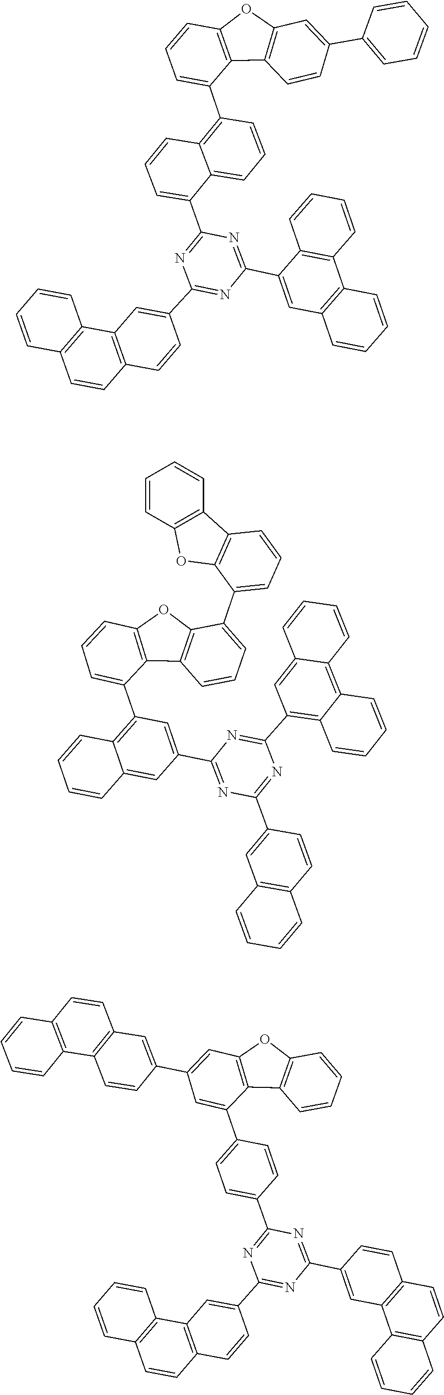 [SMILES: c1ccc(-c2ccc3c(c2)oc2cccc(-c4cccc5c(-c6nc(-c7ccc8ccc9ccccc9c8c7)nc(-c7cc8ccccc8c8ccccc78)n6)cccc45)c23)cc1 ; c1ccc2cc(-c3nc(-c4cc(-c5cccc6oc7c(-c8cccc9c8oc8ccccc89)cccc7c56)c5ccccc5c4)nc(-c4cc5ccccc5c5ccccc45)n3)ccc2c1 ; c1ccc2c(c1)ccc1cc(-c3cc(-c4ccc(-c5nc(-c6ccc7ccc8ccccc8c7c6)nc(-c6ccc7ccc8ccccc8c7c6)n5)cc4)c4c(c3)oc3ccccc34)ccc12]

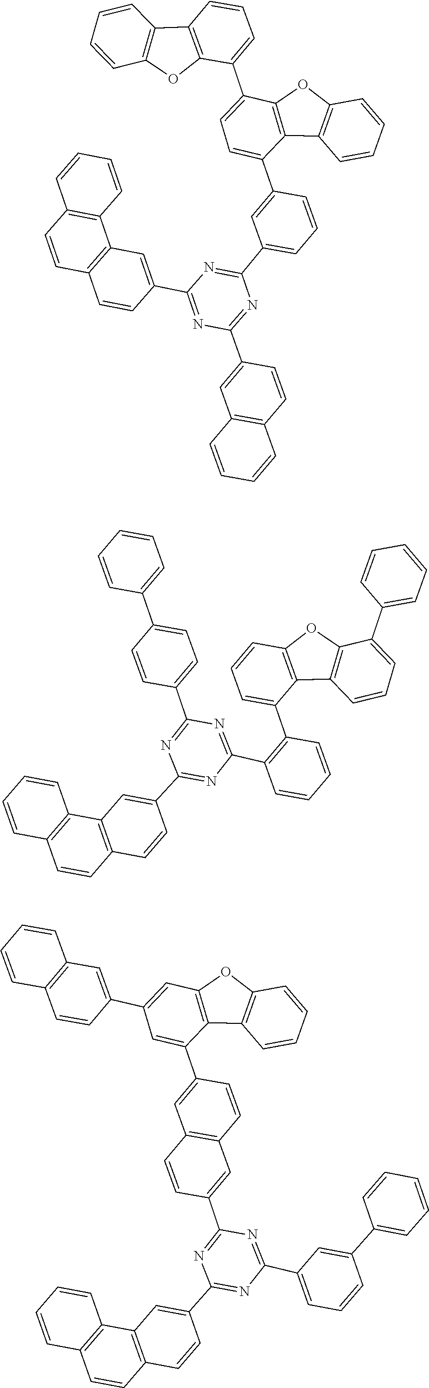 [SMILES: c1cc(-c2nc(-c3ccc4ccccc4c3)nc(-c3ccc4ccc5ccccc5c4c3)n2)cc(-c2ccc(-c3cccc4c3oc3ccccc34)c3oc4ccccc4c23)c1 ; c1ccc(-c2ccc(-c3nc(-c4ccc5ccc6ccccc6c5c4)nc(-c4ccccc4-c4cccc5oc6c(-c7ccccc7)cccc6c45)n3)cc2)cc1 ; c1ccc(-c2cccc(-c3nc(-c4ccc5cc(-c6cc(-c7ccc8ccccc8c7)cc7oc8ccccc8c67)ccc5c4)nc(-c4ccc5ccc6ccccc6c5c4)n3)c2)cc1]

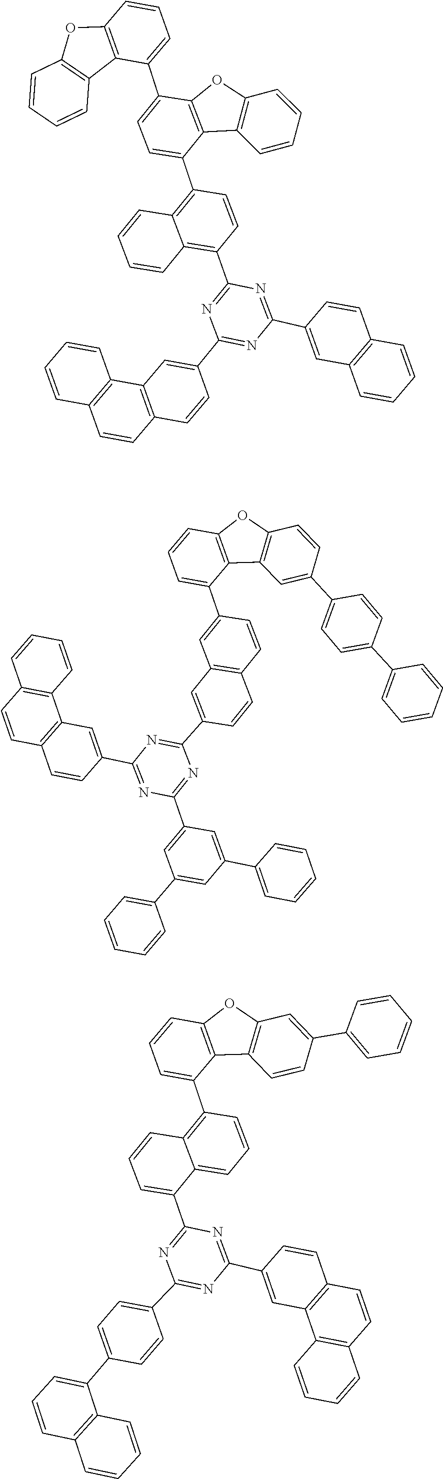 [SMILES: c1ccc2cc(-c3nc(-c4ccc5ccc6ccccc6c5c4)nc(-c4ccc(-c5ccc(-c6cccc7oc8ccccc8c67)c6oc7ccccc7c56)c5ccccc45)n3)ccc2c1 ; c1ccc(-c2ccc(-c3ccc4oc5cccc(-c6ccc7ccc(-c8nc(-c9cc(-c%10ccccc%10)cc(-c%10ccccc%10)c9)nc(-c9ccc%10ccc%11ccccc%11c%10c9)n8)cc7c6)c5c4c3)cc2)cc1 ; c1ccc(-c2ccc3c(c2)oc2cccc(-c4cccc5c(-c6nc(-c7ccc(-c8cccc9ccccc89)cc7)nc(-c7ccc8ccc9ccccc9c8c7)n6)cccc45)c23)cc1]

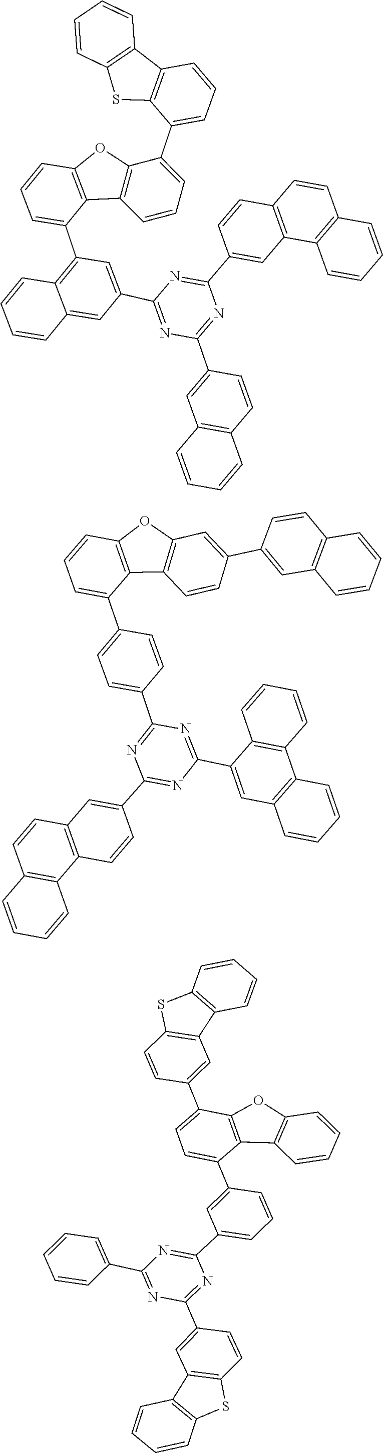

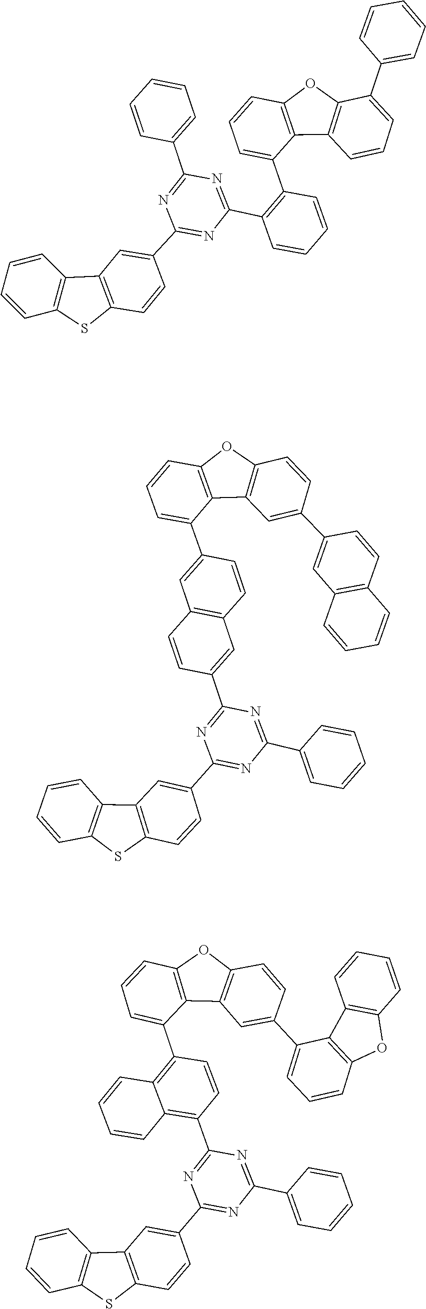 [SMILES: c1ccc(-c2nc(-c3ccc4sc5ccccc5c4c3)nc(-c3ccccc3-c3cccc4oc5c(-c6ccccc6)cccc5c34)n2)cc1 ; c1ccc(-c2nc(-c3ccc4cc(-c5cccc6oc7ccc(-c8ccc9ccccc9c8)cc7c56)ccc4c3)nc(-c3ccc4sc5ccccc5c4c3)n2)cc1 ; c1ccc(-c2nc(-c3ccc4sc5ccccc5c4c3)nc(-c3ccc(-c4cccc5oc6ccc(-c7cccc8oc9ccccc9c78)cc6c45)c4ccccc34)n2)cc1]

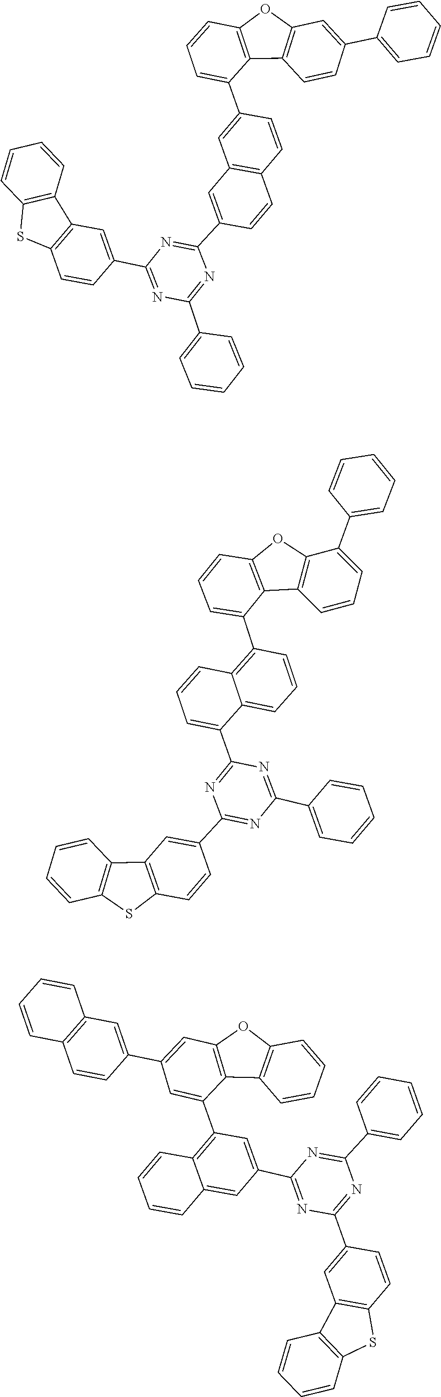 [SMILES: c1ccc(-c2ccc3c(c2)oc2cccc(-c4ccc5ccc(-c6nc(-c7ccccc7)nc(-c7ccc8sc9ccccc9c8c7)n6)cc5c4)c23)cc1 ; c1ccc(-c2nc(-c3ccc4sc5ccccc5c4c3)nc(-c3cccc4c(-c5cccc6oc7c(-c8ccccc8)cccc7c56)cccc34)n2)cc1 ; c1ccc(-c2nc(-c3cc(-c4cc(-c5ccc6ccccc6c5)cc5oc6ccccc6c45)c4ccccc4c3)nc(-c3ccc4sc5ccccc5c4c3)n2)cc1]

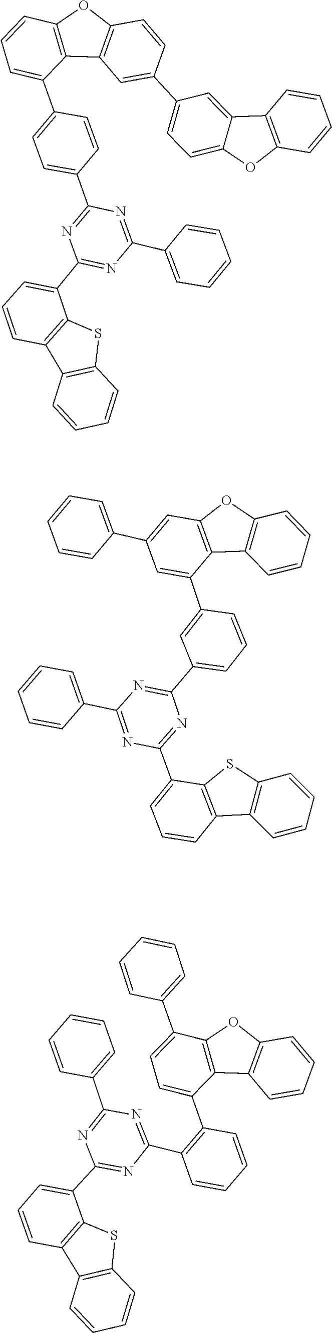 [SMILES: c1ccc(-c2nc(-c3ccc(-c4cccc5oc6ccc(-c7ccc8oc9ccccc9c8c7)cc6c45)cc3)nc(-c3cccc4c3sc3ccccc34)n2)cc1 ; c1ccc(-c2cc(-c3cccc(-c4nc(-c5ccccc5)nc(-c5cccc6c5sc5ccccc56)n4)c3)c3c(c2)oc2ccccc23)cc1 ; c1ccc(-c2nc(-c3ccccc3-c3ccc(-c4ccccc4)c4oc5ccccc5c34)nc(-c3cccc4c3sc3ccccc34)n2)cc1]

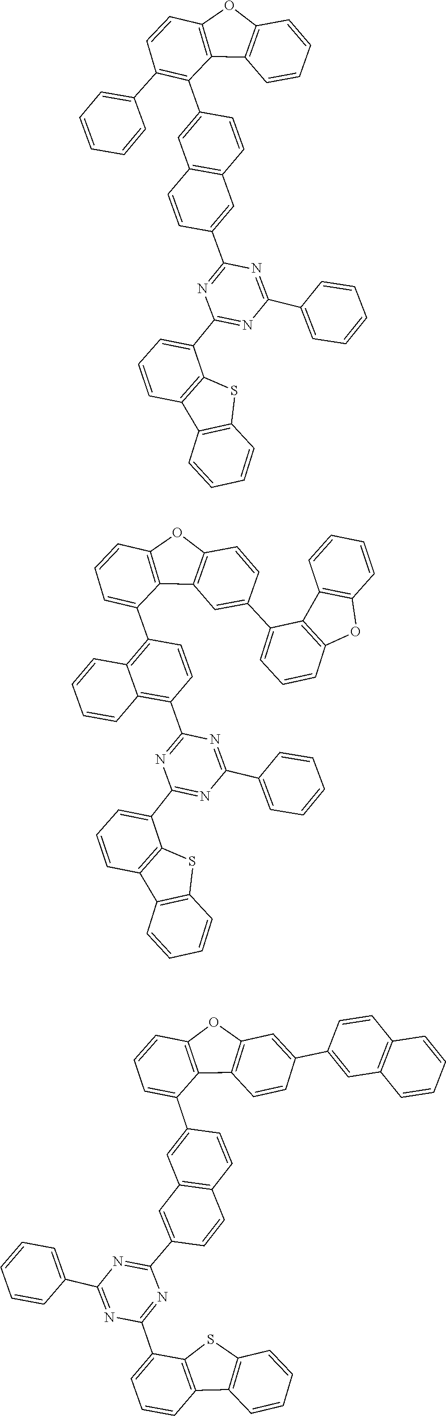 [SMILES: c1ccc(-c2nc(-c3ccc4cc(-c5c(-c6ccccc6)ccc6oc7ccccc7c56)ccc4c3)nc(-c3cccc4c3sc3ccccc34)n2)cc1 ; c1ccc(-c2nc(-c3ccc(-c4cccc5oc6ccc(-c7cccc8oc9ccccc9c78)cc6c45)c4ccccc34)nc(-c3cccc4c3sc3ccccc34)n2)cc1 ; c1ccc(-c2nc(-c3ccc4ccc(-c5cccc6oc7cc(-c8ccc9ccccc9c8)ccc7c56)cc4c3)nc(-c3cccc4c3sc3ccccc34)n2)cc1]

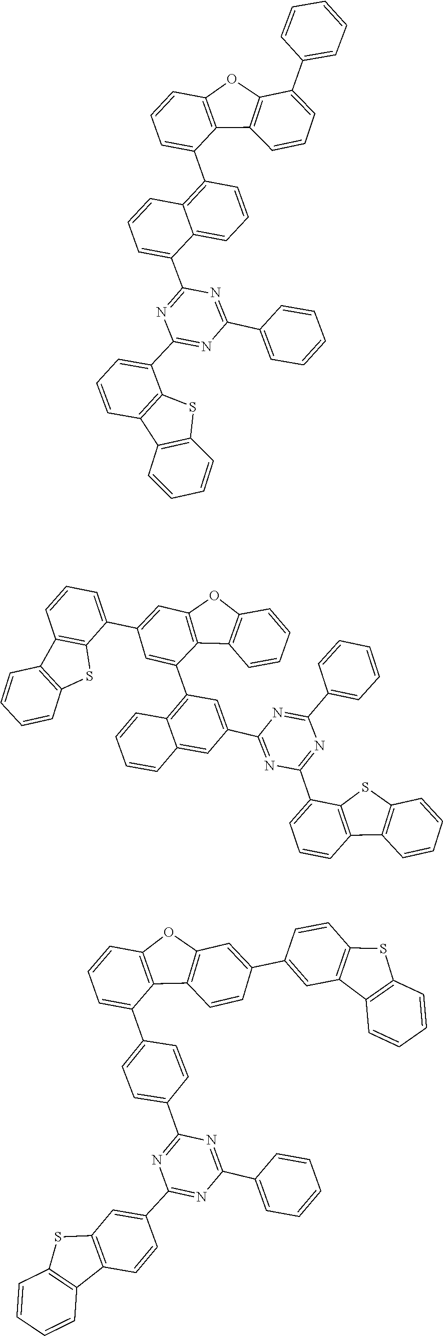 [SMILES: c1ccc(-c2nc(-c3cccc4c(-c5cccc6oc7c(-c8ccccc8)cccc7c56)cccc34)nc(-c3cccc4c3sc3ccccc34)n2)cc1 ; c1ccc(-c2nc(-c3cc(-c4cc(-c5cccc6c5sc5ccccc56)cc5oc6ccccc6c45)c4ccccc4c3)nc(-c3cccc4c3sc3ccccc34)n2)cc1 ; c1ccc(-c2nc(-c3ccc(-c4cccc5oc6cc(-c7ccc8sc9ccccc9c8c7)ccc6c45)cc3)nc(-c3ccc4c(c3)sc3ccccc34)n2)cc1]

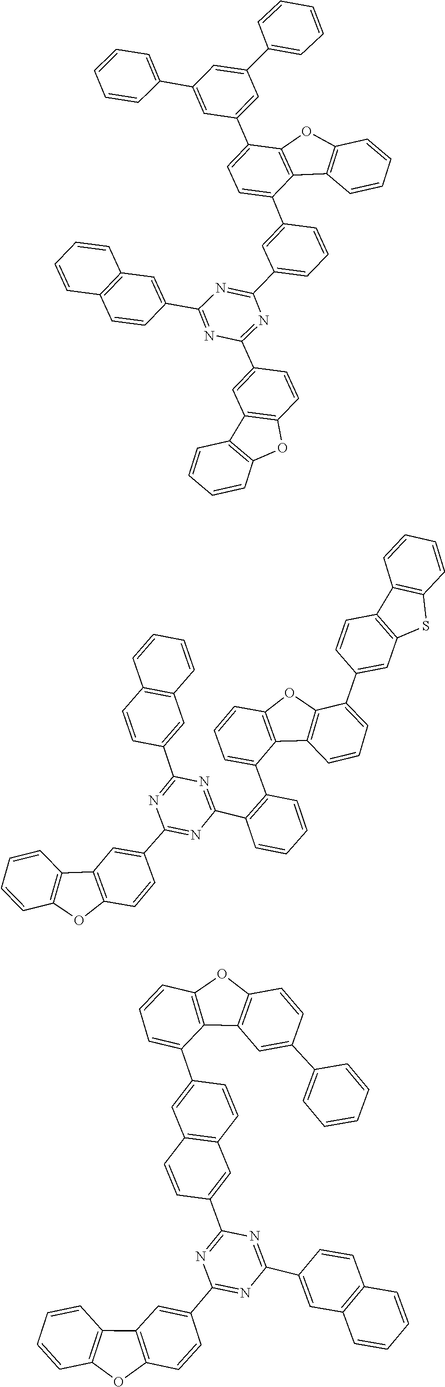 [SMILES: c1ccc(-c2cc(-c3ccccc3)cc(-c3ccc(-c4cccc(-c5nc(-c6ccc7ccccc7c6)nc(-c6ccc7oc8ccccc8c7c6)n5)c4)c4c3oc3ccccc34)c2)cc1 ; c1ccc(-c2cccc3oc4c(-c5ccc6c(c5)sc5ccccc56)cccc4c23)c(-c2nc(-c3ccc4ccccc4c3)nc(-c3ccc4oc5ccccc5c4c3)n2)c1 ; c1ccc(-c2ccc3oc4cccc(-c5ccc6cc(-c7nc(-c8ccc9ccccc9c8)nc(-c8ccc9oc%10ccccc%10c9c8)n7)ccc6c5)c4c3c2)cc1]

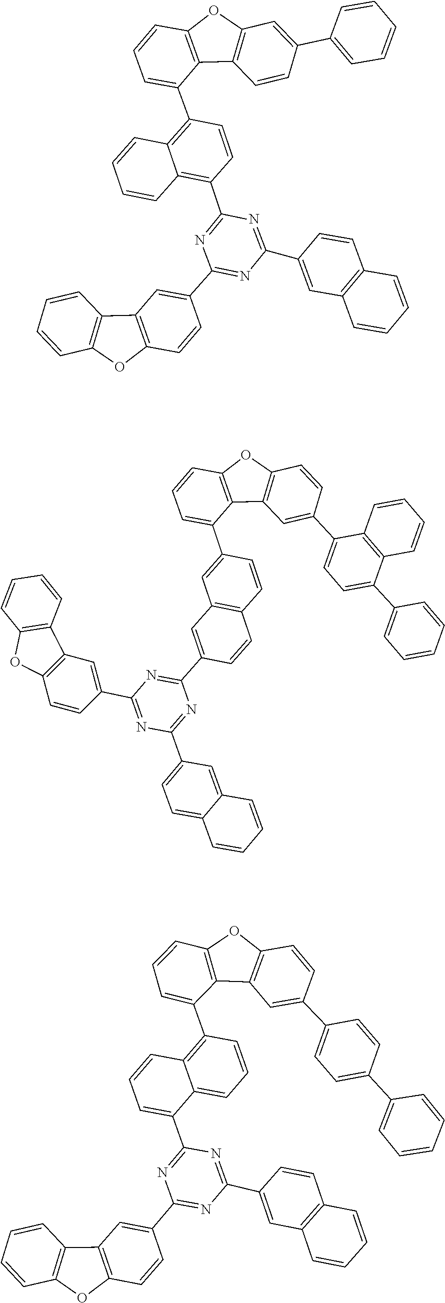 [SMILES: c1ccc(-c2ccc3c(c2)oc2cccc(-c4ccc(-c5nc(-c6ccc7ccccc7c6)nc(-c6ccc7oc8ccccc8c7c6)n5)c5ccccc45)c23)cc1 ; c1ccc(-c2ccc(-c3ccc4oc5cccc(-c6ccc7ccc(-c8nc(-c9ccc%10ccccc%10c9)nc(-c9ccc%10oc%11ccccc%11c%10c9)n8)cc7c6)c5c4c3)c3ccccc23)cc1 ; c1ccc(-c2ccc(-c3ccc4oc5cccc(-c6cccc7c(-c8nc(-c9ccc%10ccccc%10c9)nc(-c9ccc%10oc%11ccccc%11c%10c9)n8)cccc67)c5c4c3)cc2)cc1]

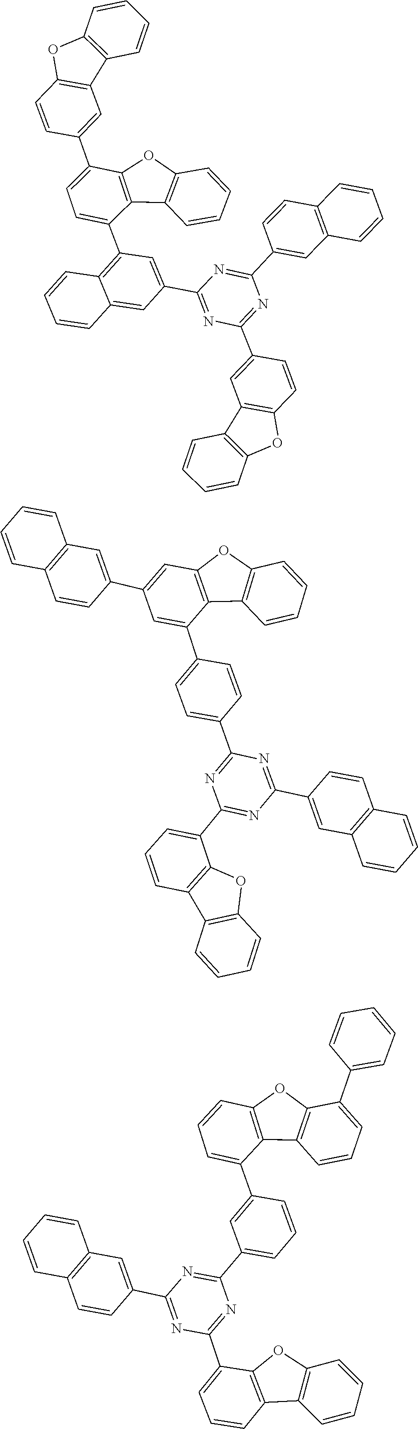 [SMILES: c1ccc2cc(-c3nc(-c4cc(-c5ccc(-c6ccc7oc8ccccc8c7c6)c6oc7ccccc7c56)c5ccccc5c4)nc(-c4ccc5oc6ccccc6c5c4)n3)ccc2c1 ; c1ccc2cc(-c3cc(-c4ccc(-c5nc(-c6ccc7ccccc7c6)nc(-c6cccc7c6oc6ccccc67)n5)cc4)c4c(c3)oc3ccccc34)ccc2c1 ; c1ccc(-c2cccc3c2oc2cccc(-c4cccc(-c5nc(-c6ccc7ccccc7c6)nc(-c6cccc7c6oc6ccccc67)n5)c4)c23)cc1]

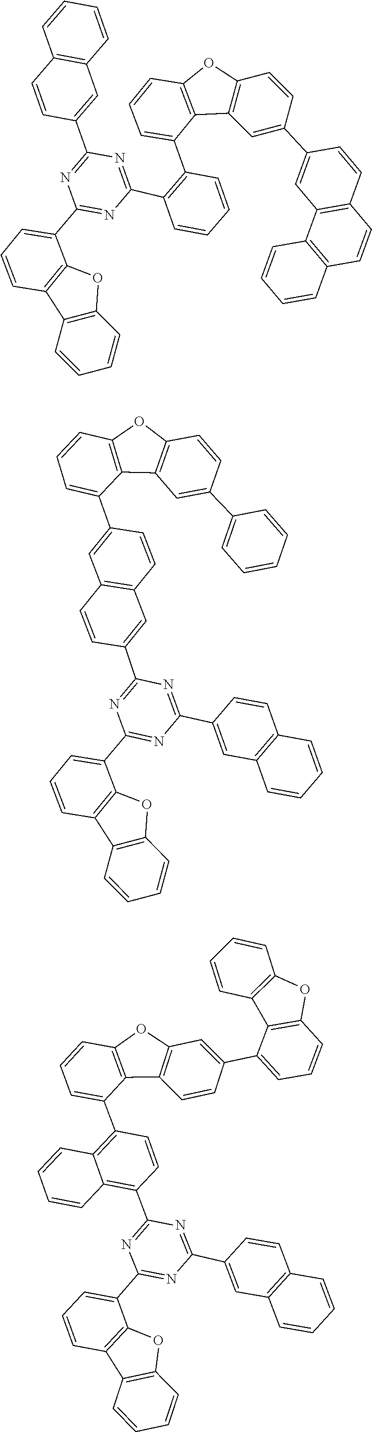 [SMILES: c1ccc(-c2cccc3oc4ccc(-c5ccc6ccc7ccccc7c6c5)cc4c23)c(-c2nc(-c3ccc4ccccc4c3)nc(-c3cccc4c3oc3ccccc34)n2)c1 ; c1ccc(-c2ccc3oc4cccc(-c5ccc6cc(-c7nc(-c8ccc9ccccc9c8)nc(-c8cccc9c8oc8ccccc89)n7)ccc6c5)c4c3c2)cc1 ; c1ccc2cc(-c3nc(-c4ccc(-c5cccc6oc7cc(-c8cccc9oc%10ccccc%10c89)ccc7c56)c5ccccc45)nc(-c4cccc5c4oc4ccccc45)n3)ccc2c1]

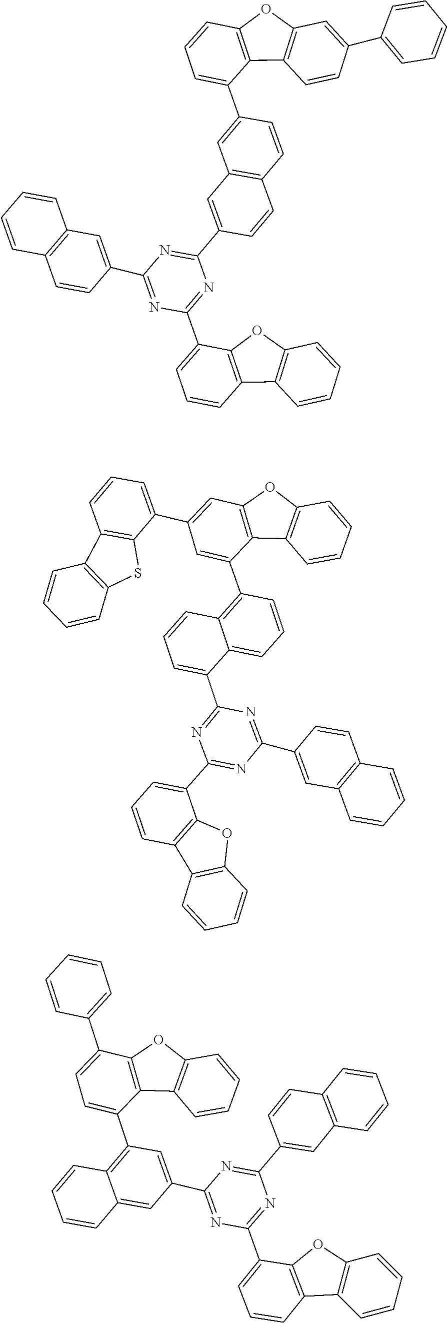 [SMILES: c1ccc(-c2ccc3c(c2)oc2cccc(-c4ccc5ccc(-c6nc(-c7ccc8ccccc8c7)nc(-c7cccc8c7oc7ccccc78)n6)cc5c4)c23)cc1 ; c1ccc2cc(-c3nc(-c4cccc5c(-c6cc(-c7cccc8c7sc7ccccc78)cc7oc8ccccc8c67)cccc45)nc(-c4cccc5c4oc4ccccc45)n3)ccc2c1 ; c1ccc(-c2ccc(-c3cc(-c4nc(-c5ccc6ccccc6c5)nc(-c5cccc6c5oc5ccccc56)n4)cc4ccccc34)c3c2oc2ccccc23)cc1]

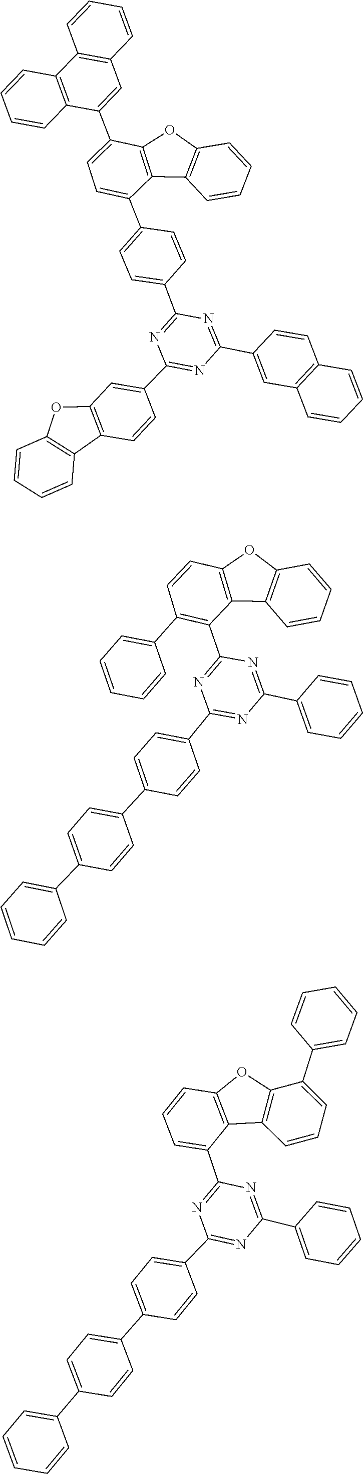 [SMILES: c1ccc2cc(-c3nc(-c4ccc(-c5ccc(-c6cc7ccccc7c7ccccc67)c6oc7ccccc7c56)cc4)nc(-c4ccc5c(c4)oc4ccccc45)n3)ccc2c1 ; c1ccc(-c2ccc(-c3ccc(-c4nc(-c5ccccc5)nc(-c5c(-c6ccccc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccccc8)cccc7c56)n4)cc3)cc2)cc1]

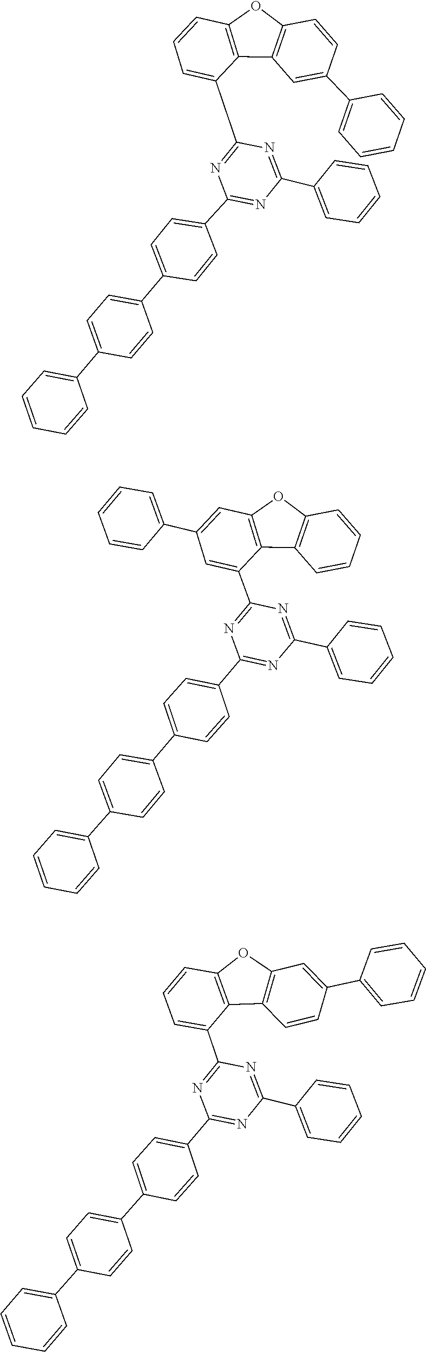 [SMILES: c1ccc(-c2ccc(-c3ccc(-c4nc(-c5ccccc5)nc(-c5cccc6oc7ccc(-c8ccccc8)cc7c56)n4)cc3)cc2)cc1 ; c1ccc(-c2ccc(-c3ccc(-c4nc(-c5ccccc5)nc(-c5cc(-c6ccccc6)cc6oc7ccccc7c56)n4)cc3)cc2)cc1 ; c1ccc(-c2ccc(-c3ccc(-c4nc(-c5ccccc5)nc(-c5cccc6oc7cc(-c8ccccc8)ccc7c56)n4)cc3)cc2)cc1]

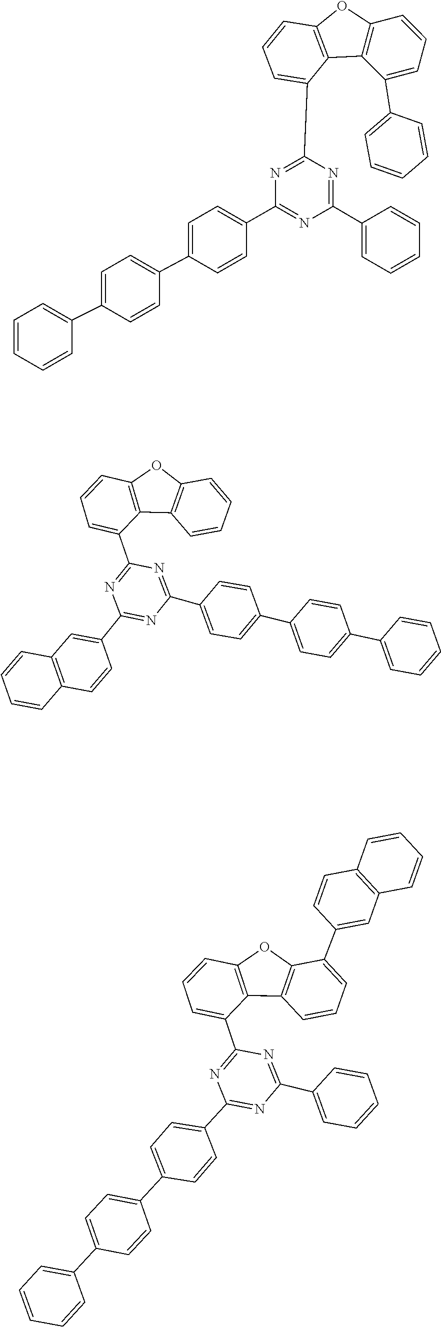 [SMILES: c1ccc(-c2ccc(-c3ccc(-c4nc(-c5ccccc5)nc(-c5cccc6oc7cccc(-c8ccccc8)c7c56)n4)cc3)cc2)cc1 ; c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3ccc(-c4nc(-c5ccccc5)nc(-c5cccc6oc7c(-c8ccc9ccccc9c8)cccc7c56)n4)cc3)cc2)cc1]

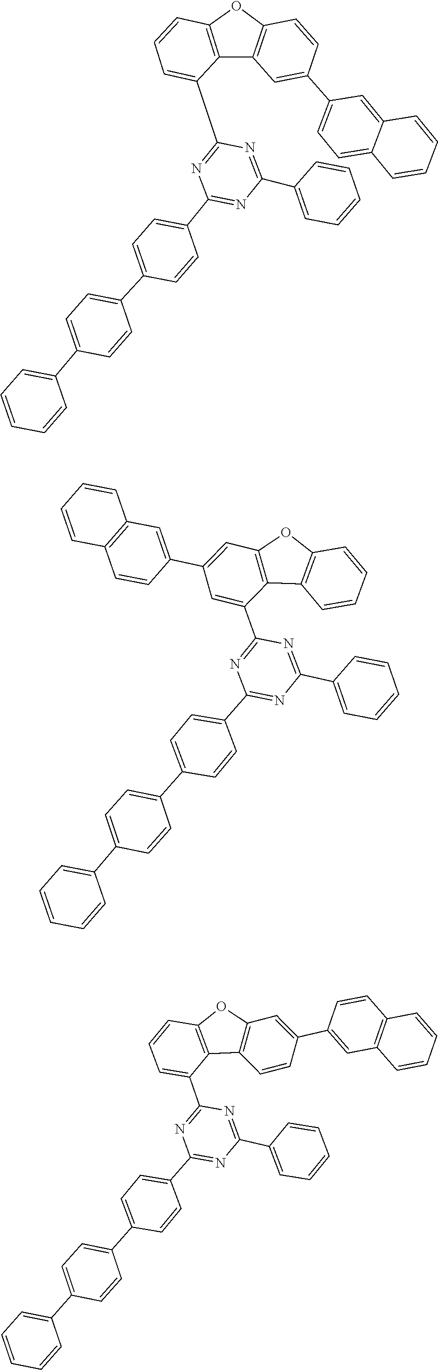 [SMILES: c1ccc(-c2ccc(-c3ccc(-c4nc(-c5ccccc5)nc(-c5cccc6oc7ccc(-c8ccc9ccccc9c8)cc7c56)n4)cc3)cc2)cc1 ; c1ccc(-c2ccc(-c3ccc(-c4nc(-c5ccccc5)nc(-c5cc(-c6ccc7ccccc7c6)cc6oc7ccccc7c56)n4)cc3)cc2)cc1 ; c1ccc(-c2ccc(-c3ccc(-c4nc(-c5ccccc5)nc(-c5cccc6oc7cc(-c8ccc9ccccc9c8)ccc7c56)n4)cc3)cc2)cc1]

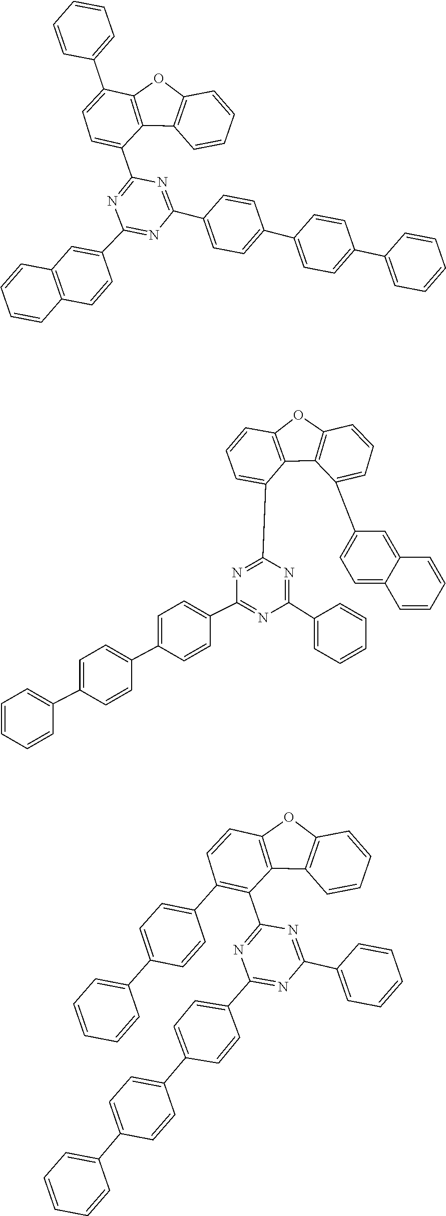 [SMILES: c1ccc(-c2ccc(-c3ccc(-c4nc(-c5ccc6ccccc6c5)nc(-c5ccc(-c6ccccc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9ccccc9c8)c7c56)n4)cc3)cc2)cc1 ; c1ccc(-c2ccc(-c3ccc(-c4nc(-c5ccccc5)nc(-c5c(-c6ccc(-c7ccccc7)cc6)ccc6oc7ccccc7c56)n4)cc3)cc2)cc1]

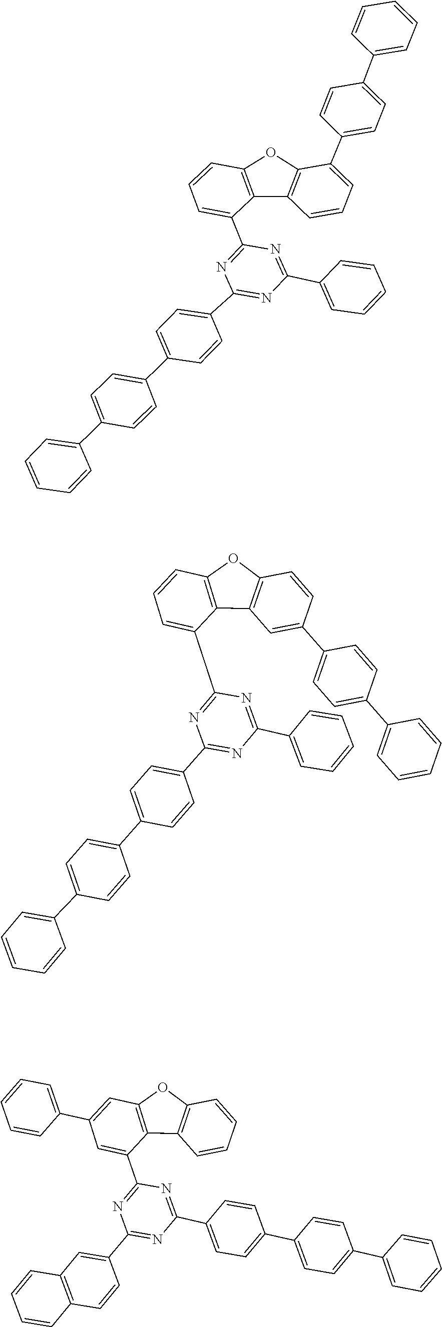 [SMILES: c1ccc(-c2ccc(-c3ccc(-c4nc(-c5ccccc5)nc(-c5cccc6oc7c(-c8ccc(-c9ccccc9)cc8)cccc7c56)n4)cc3)cc2)cc1 ; c1ccc(-c2ccc(-c3ccc(-c4nc(-c5ccccc5)nc(-c5cccc6oc7ccc(-c8ccc(-c9ccccc9)cc8)cc7c56)n4)cc3)cc2)cc1 ; c1ccc(-c2ccc(-c3ccc(-c4nc(-c5ccc6ccccc6c5)nc(-c5cc(-c6ccccc6)cc6oc7ccccc7c56)n4)cc3)cc2)cc1]

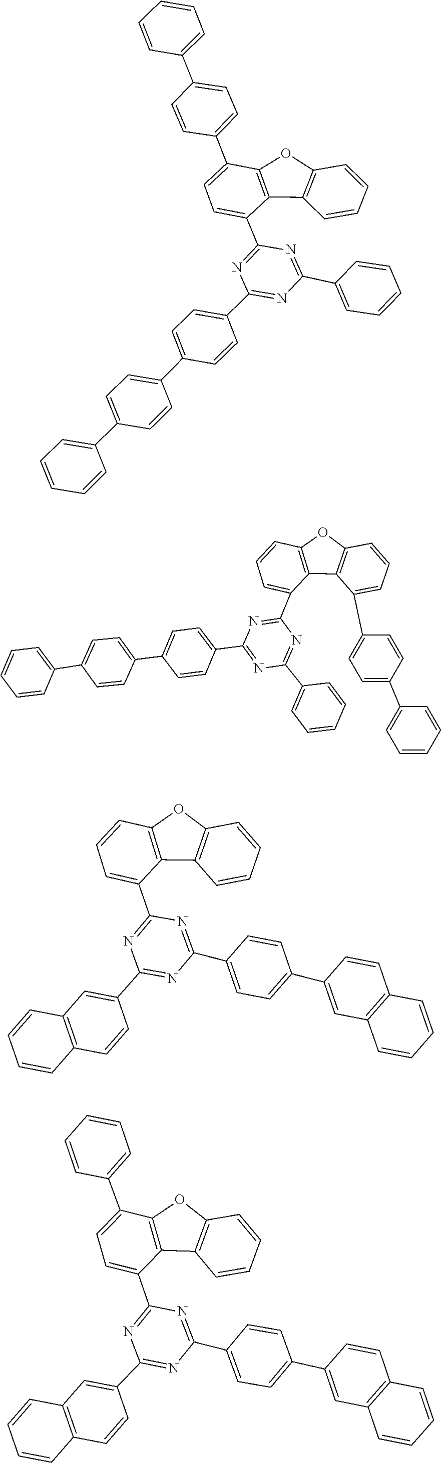 [SMILES: c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1 ; c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3nc(-c4ccc(-c5ccc6ccccc6c5)cc4)nc(-c4ccc5ccccc5c4)n3)c3c2oc2ccccc23)cc1]

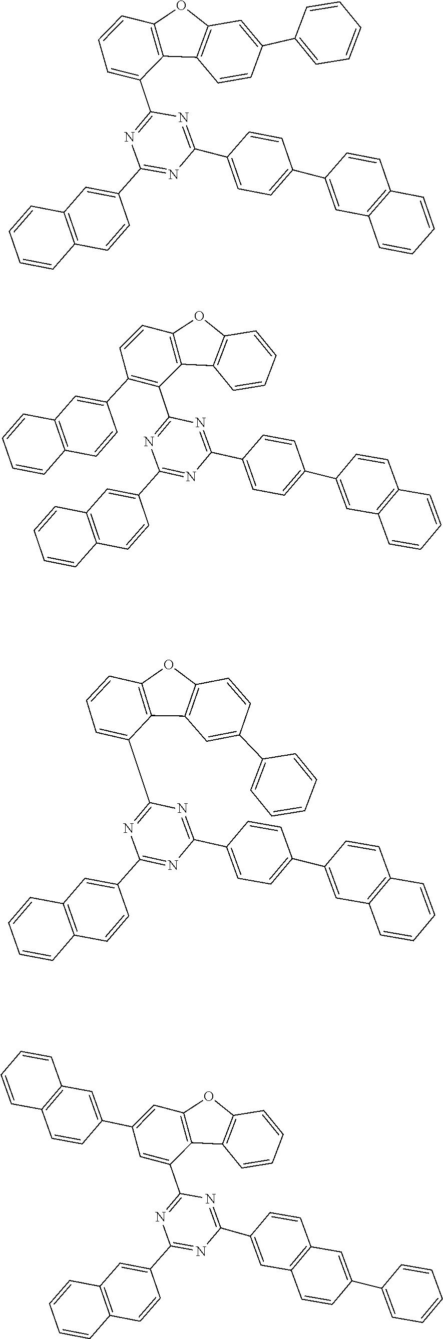 [SMILES: c1ccc(-c2ccc3c(c2)oc2cccc(-c4nc(-c5ccc(-c6ccc7ccccc7c6)cc5)nc(-c5ccc6ccccc6c5)n4)c23)cc1 ; c1ccc2cc(-c3ccc(-c4nc(-c5ccc6ccccc6c5)nc(-c5c(-c6ccc7ccccc7c6)ccc6oc7ccccc7c56)n4)cc3)ccc2c1 ; c1ccc(-c2ccc3oc4cccc(-c5nc(-c6ccc(-c7ccc8ccccc8c7)cc6)nc(-c6ccc7ccccc7c6)n5)c4c3c2)cc1 ; c1ccc(-c2ccc3cc(-c4nc(-c5ccc6ccccc6c5)nc(-c5cc(-c6ccc7ccccc7c6)cc6oc7ccccc7c56)n4)ccc3c2)cc1]

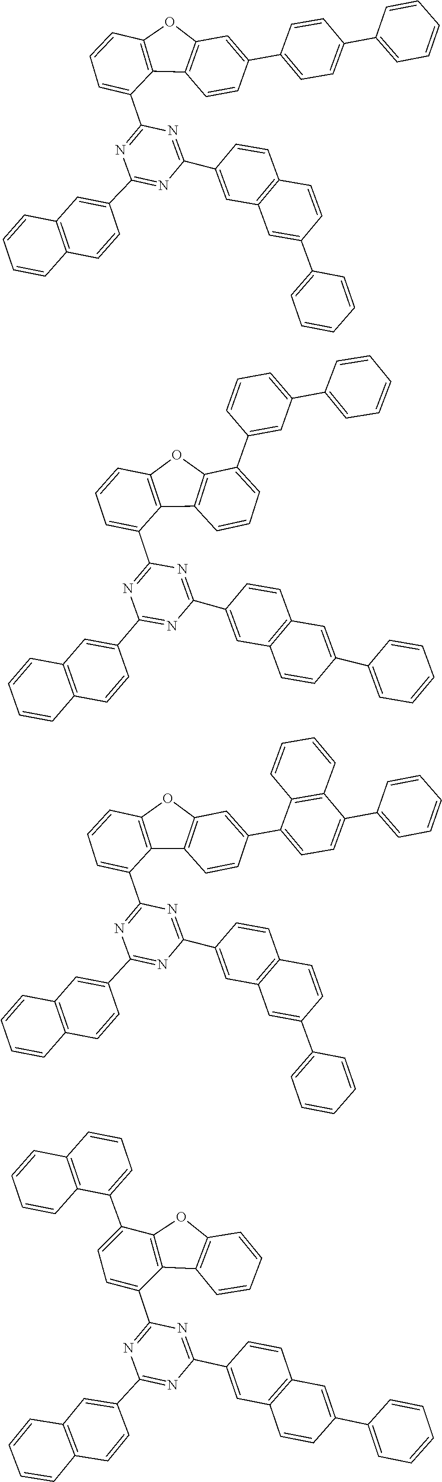

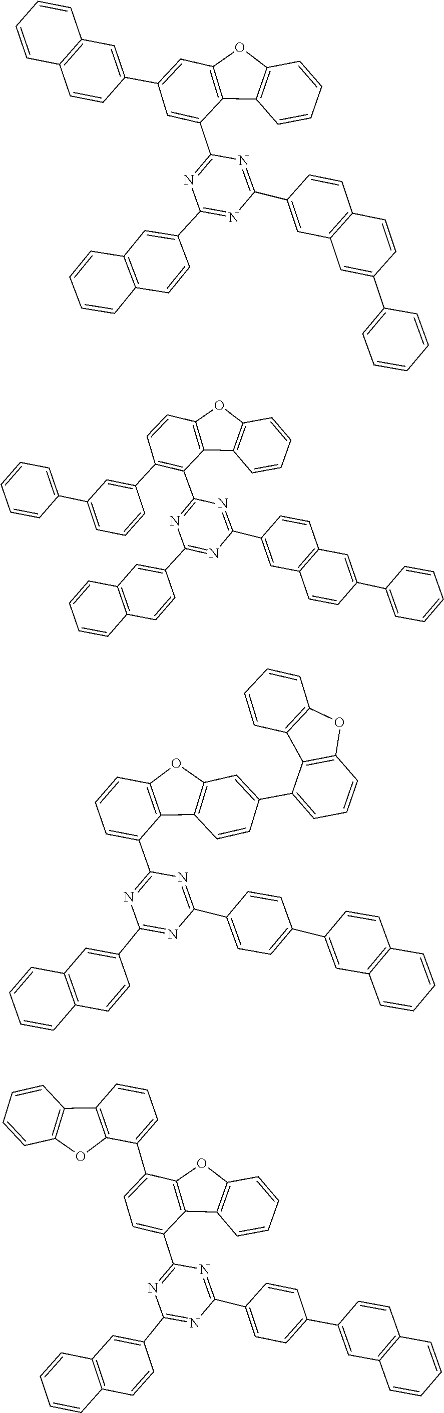 [SMILES: c1ccc(-c2ccc3ccc(-c4nc(-c5ccc6ccccc6c5)nc(-c5cc(-c6ccc7ccccc7c6)cc6oc7ccccc7c56)n4)cc3c2)cc1 ; c1ccc(-c2cccc(-c3ccc4oc5ccccc5c4c3-c3nc(-c4ccc5ccccc5c4)nc(-c4ccc5cc(-c6ccccc6)ccc5c4)n3)c2)cc1 ; c1ccc2cc(-c3ccc(-c4nc(-c5ccc6ccccc6c5)nc(-c5cccc6oc7cc(-c8cccc9oc%10ccccc%10c89)ccc7c56)n4)cc3)ccc2c1 ; c1ccc2cc(-c3ccc(-c4nc(-c5ccc6ccccc6c5)nc(-c5ccc(-c6cccc7c6oc6ccccc67)c6oc7ccccc7c56)n4)cc3)ccc2c1]

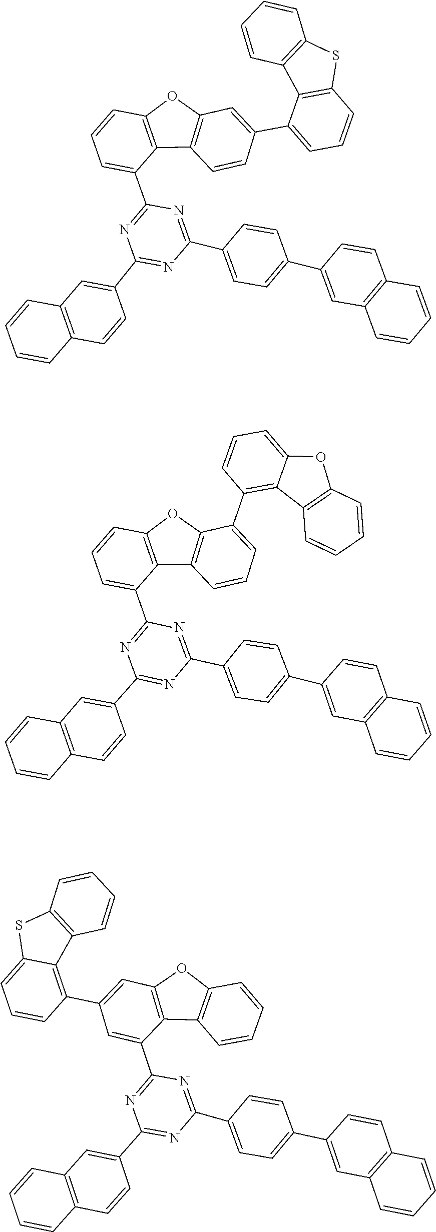 [SMILES: c1ccc2cc(-c3ccc(-c4nc(-c5ccc6ccccc6c5)nc(-c5cccc6oc7cc(-c8cccc9sc%10ccccc%10c89)ccc7c56)n4)cc3)ccc2c1 ; c1ccc2cc(-c3ccc(-c4nc(-c5ccc6ccccc6c5)nc(-c5cccc6oc7c(-c8cccc9oc%10ccccc%10c89)cccc7c56)n4)cc3)ccc2c1 ; c1ccc2cc(-c3ccc(-c4nc(-c5ccc6ccccc6c5)nc(-c5cc(-c6cccc7sc8ccccc8c67)cc6oc7ccccc7c56)n4)cc3)ccc2c1]

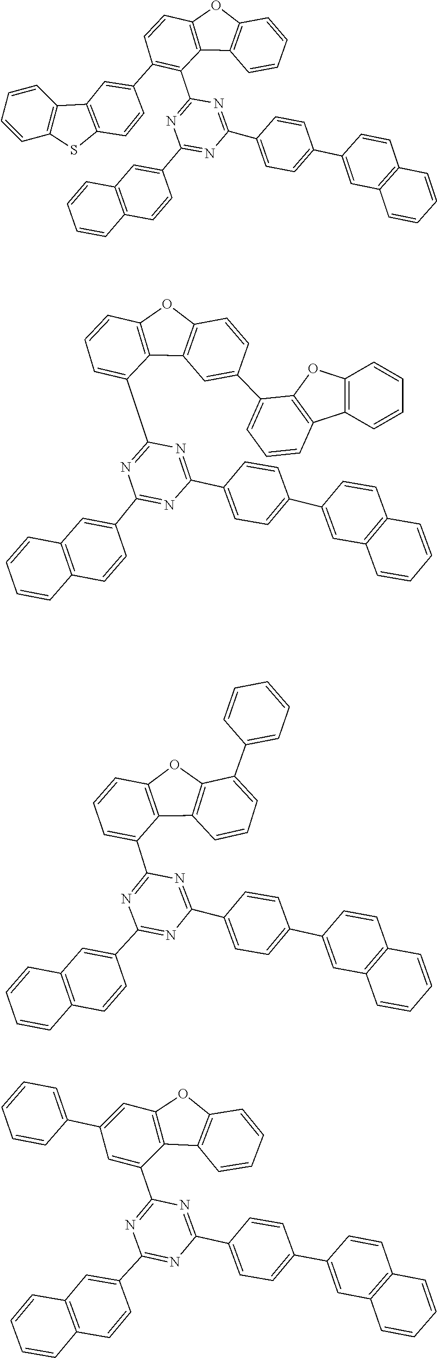 [SMILES: c1ccc2cc(-c3ccc(-c4nc(-c5ccc6ccccc6c5)nc(-c5c(-c6ccc7sc8ccccc8c7c6)ccc6oc7ccccc7c56)n4)cc3)ccc2c1 ; c1ccc2cc(-c3ccc(-c4nc(-c5ccc6ccccc6c5)nc(-c5cccc6oc7ccc(-c8cccc9c8oc8ccccc89)cc7c56)n4)cc3)ccc2c1 ; c1ccc(-c2cccc3c2oc2cccc(-c4nc(-c5ccc(-c6ccc7ccccc7c6)cc5)nc(-c5ccc6ccccc6c5)n4)c23)cc1 ; c1ccc(-c2cc(-c3nc(-c4ccc(-c5ccc6ccccc6c5)cc4)nc(-c4ccc5ccccc5c4)n3)c3c(c2)oc2ccccc23)cc1]

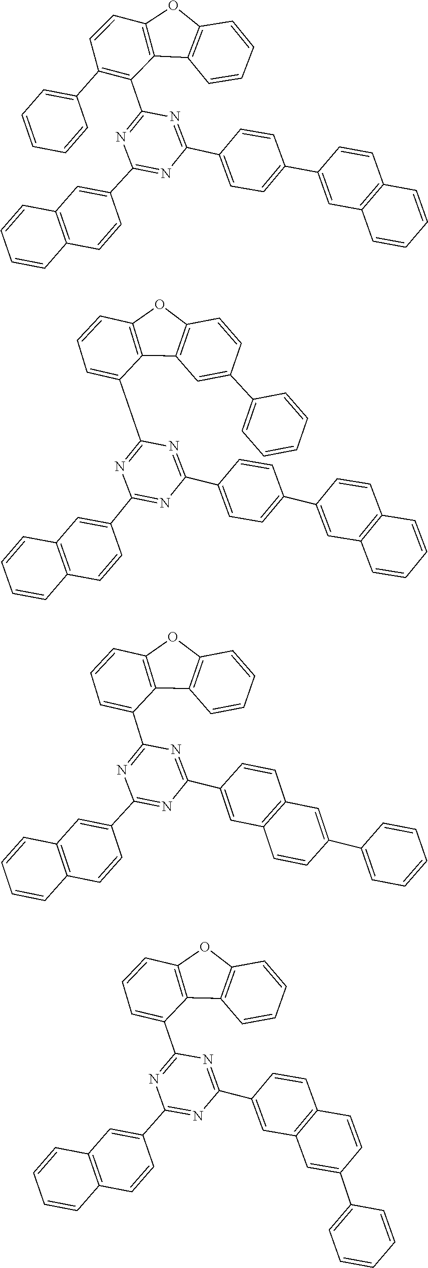 [SMILES: c1ccc(-c2ccc3oc4ccccc4c3c2-c2nc(-c3ccc(-c4ccc5ccccc5c4)cc3)nc(-c3ccc4ccccc4c3)n2)cc1 ; c1ccc(-c2ccc3oc4cccc(-c5nc(-c6ccc(-c7ccc8ccccc8c7)cc6)nc(-c6ccc7ccccc7c6)n5)c4c3c2)cc1 ; c1ccc(-c2ccc3cc(-c4nc(-c5ccc6ccccc6c5)nc(-c5cccc6oc7ccccc7c56)n4)ccc3c2)cc1 ; c1ccc(-c2ccc3ccc(-c4nc(-c5ccc6ccccc6c5)nc(-c5cccc6oc7ccccc7c56)n4)cc3c2)cc1]

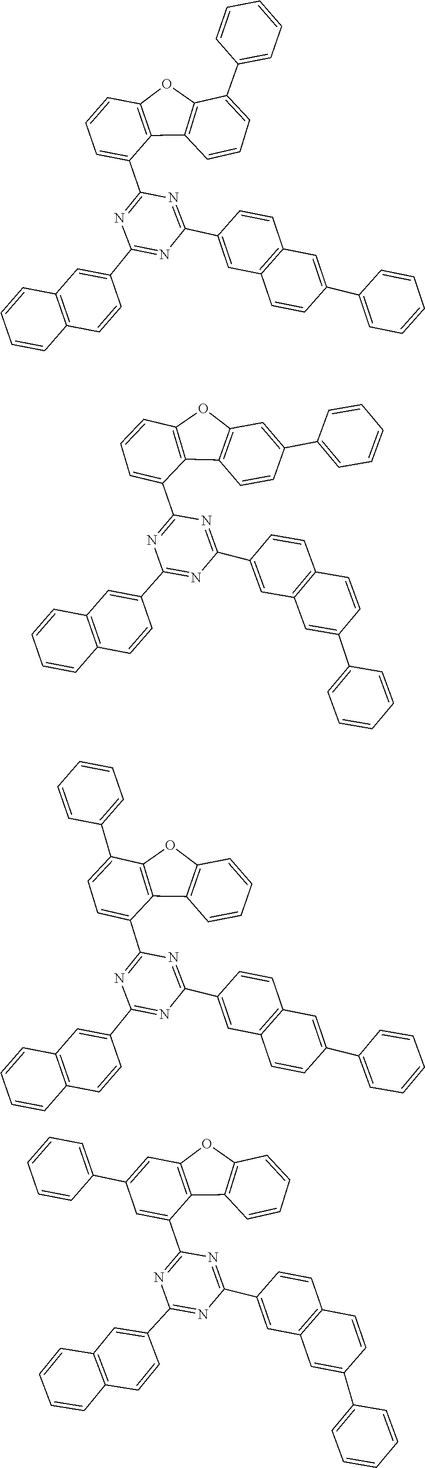

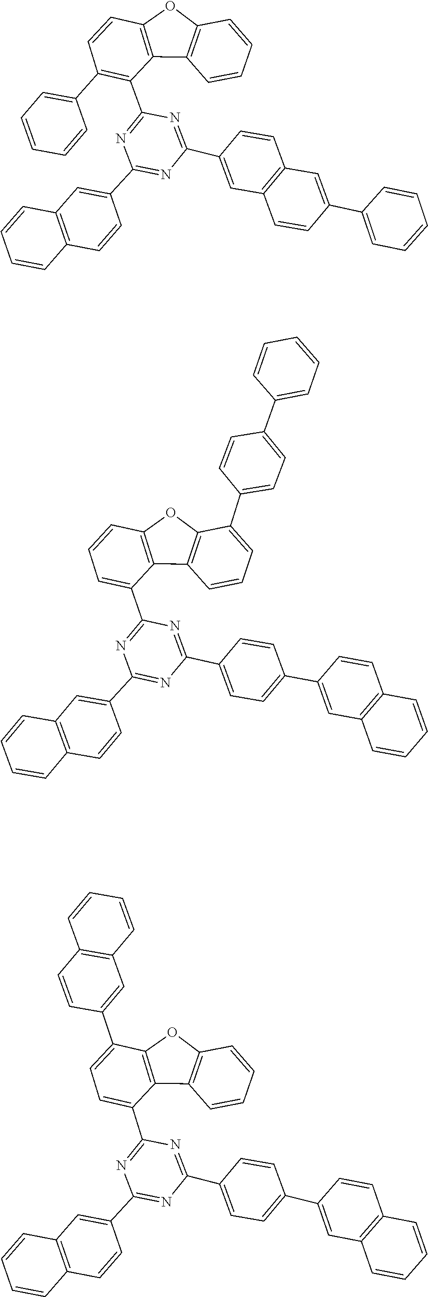 [SMILES: c1ccc(-c2ccc3cc(-c4nc(-c5ccc6ccccc6c5)nc(-c5c(-c6ccccc6)ccc6oc7ccccc7c56)n4)ccc3c2)cc1 ; c1ccc(-c2ccc(-c3cccc4c3oc3cccc(-c5nc(-c6ccc(-c7ccc8ccccc8c7)cc6)nc(-c6ccc7ccccc7c6)n5)c34)cc2)cc1 ; c1ccc2cc(-c3ccc(-c4nc(-c5ccc6ccccc6c5)nc(-c5ccc(-c6ccc7ccccc7c6)c6oc7ccccc7c56)n4)cc3)ccc2c1]

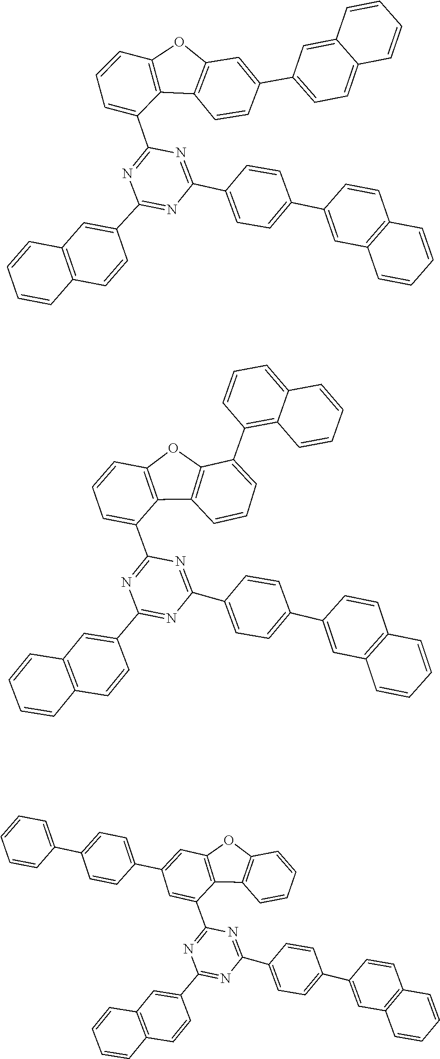 [SMILES: c1ccc2cc(-c3ccc(-c4nc(-c5ccc6ccccc6c5)nc(-c5cccc6oc7cc(-c8ccc9ccccc9c8)ccc7c56)n4)cc3)ccc2c1 ; c1ccc2cc(-c3ccc(-c4nc(-c5ccc6ccccc6c5)nc(-c5cccc6oc7c(-c8cccc9ccccc89)cccc7c56)n4)cc3)ccc2c1 ; c1ccc(-c2ccc(-c3cc(-c4nc(-c5ccc(-c6ccc7ccccc7c6)cc5)nc(-c5ccc6ccccc6c5)n4)c4c(c3)oc3ccccc34)cc2)cc1]

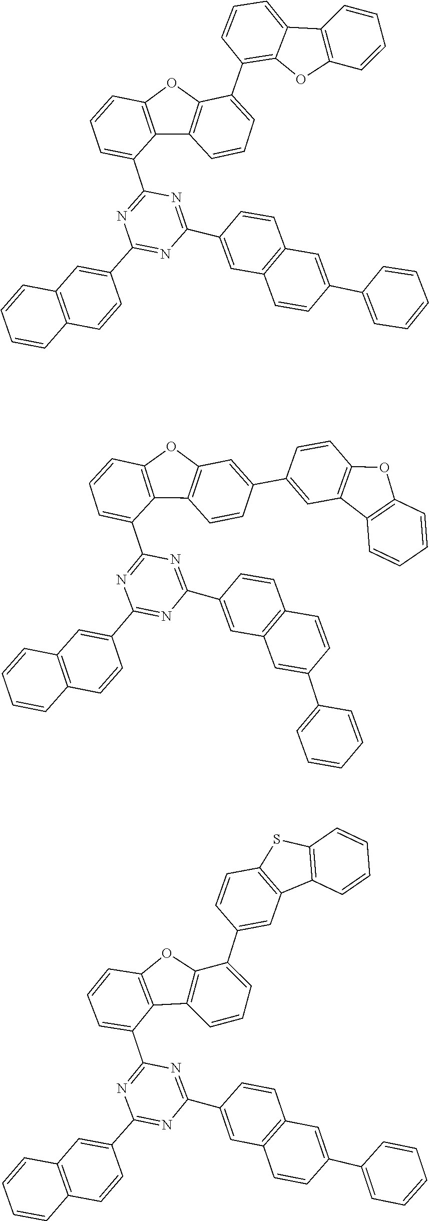 [SMILES: c1ccc(-c2ccc3cc(-c4nc(-c5ccc6ccccc6c5)nc(-c5cccc6oc7c(-c8cccc9c8oc8ccccc89)cccc7c56)n4)ccc3c2)cc1 ; c1ccc(-c2ccc3ccc(-c4nc(-c5ccc6ccccc6c5)nc(-c5cccc6oc7cc(-c8ccc9oc%10ccccc%10c9c8)ccc7c56)n4)cc3c2)cc1 ; c1ccc(-c2ccc3cc(-c4nc(-c5ccc6ccccc6c5)nc(-c5cccc6oc7c(-c8ccc9sc%10ccccc%10c9c8)cccc7c56)n4)ccc3c2)cc1]

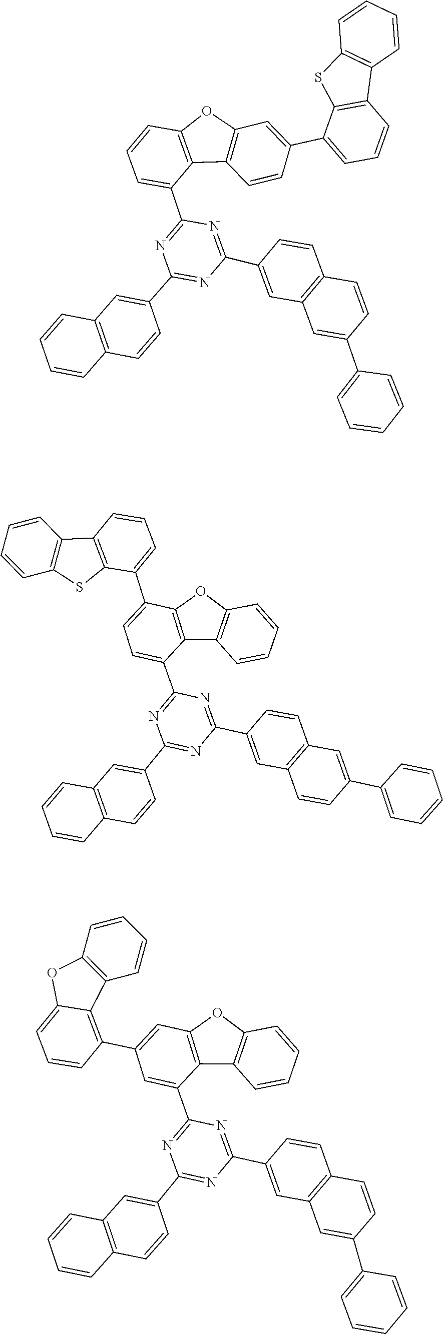 [SMILES: c1ccc(-c2ccc3ccc(-c4nc(-c5ccc6ccccc6c5)nc(-c5cccc6oc7cc(-c8cccc9c8sc8ccccc89)ccc7c56)n4)cc3c2)cc1 ; c1ccc(-c2ccc3cc(-c4nc(-c5ccc6ccccc6c5)nc(-c5ccc(-c6cccc7c6sc6ccccc67)c6oc7ccccc7c56)n4)ccc3c2)cc1 ; c1ccc(-c2ccc3ccc(-c4nc(-c5ccc6ccccc6c5)nc(-c5cc(-c6cccc7oc8ccccc8c67)cc6oc7ccccc7c56)n4)cc3c2)cc1]

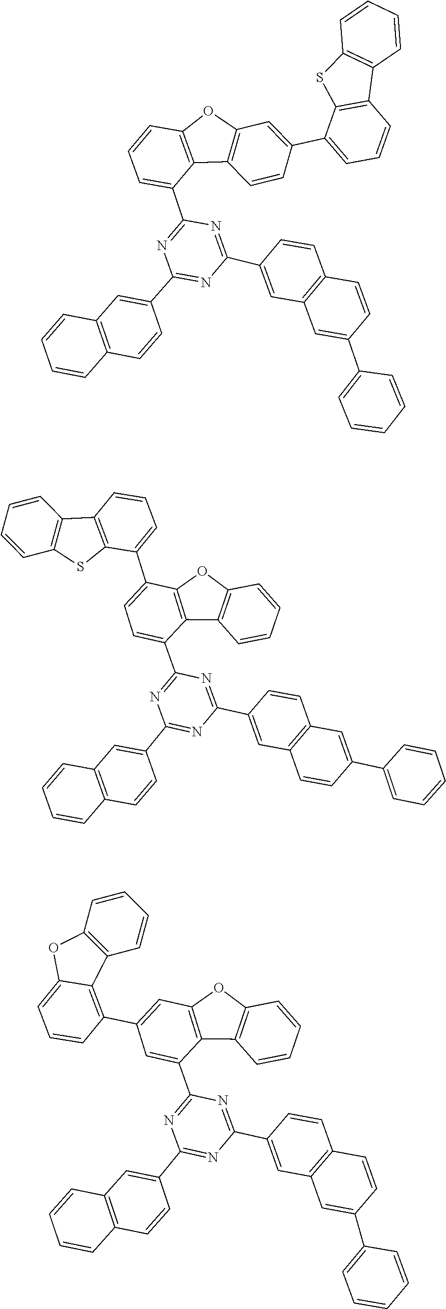 [SMILES: c1ccc(-c2ccc3ccc(-c4nc(-c5ccc6ccccc6c5)nc(-c5cccc6oc7cc(-c8cccc9c8sc8ccccc89)ccc7c56)n4)cc3c2)cc1 ; c1ccc(-c2ccc3cc(-c4nc(-c5ccc6ccccc6c5)nc(-c5ccc(-c6cccc7c6sc6ccccc67)c6oc7ccccc7c56)n4)ccc3c2)cc1 ; c1ccc(-c2ccc3ccc(-c4nc(-c5ccc6ccccc6c5)nc(-c5cc(-c6cccc7oc8ccccc8c67)cc6oc7ccccc7c56)n4)cc3c2)cc1]

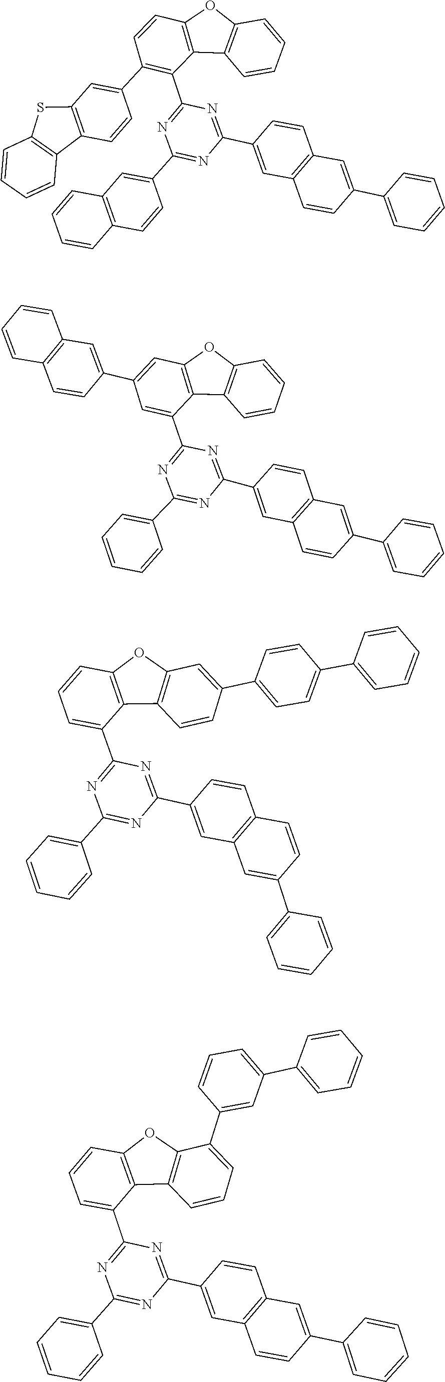 [SMILES: c1ccc(-c2ccc3cc(-c4nc(-c5ccc6ccccc6c5)nc(-c5c(-c6ccc7c(c6)sc6ccccc67)ccc6oc7ccccc7c56)n4)ccc3c2)cc1 ; c1ccc(-c2ccc3cc(-c4nc(-c5ccccc5)nc(-c5cc(-c6ccc7ccccc7c6)cc6oc7ccccc7c56)n4)ccc3c2)cc1 ; c1ccc(-c2ccc(-c3ccc4c(c3)oc3cccc(-c5nc(-c6ccccc6)nc(-c6ccc7ccc(-c8ccccc8)cc7c6)n5)c34)cc2)cc1 ; c1ccc(-c2cccc(-c3cccc4c3oc3cccc(-c5nc(-c6ccccc6)nc(-c6ccc7cc(-c8ccccc8)ccc7c6)n5)c34)c2)cc1]

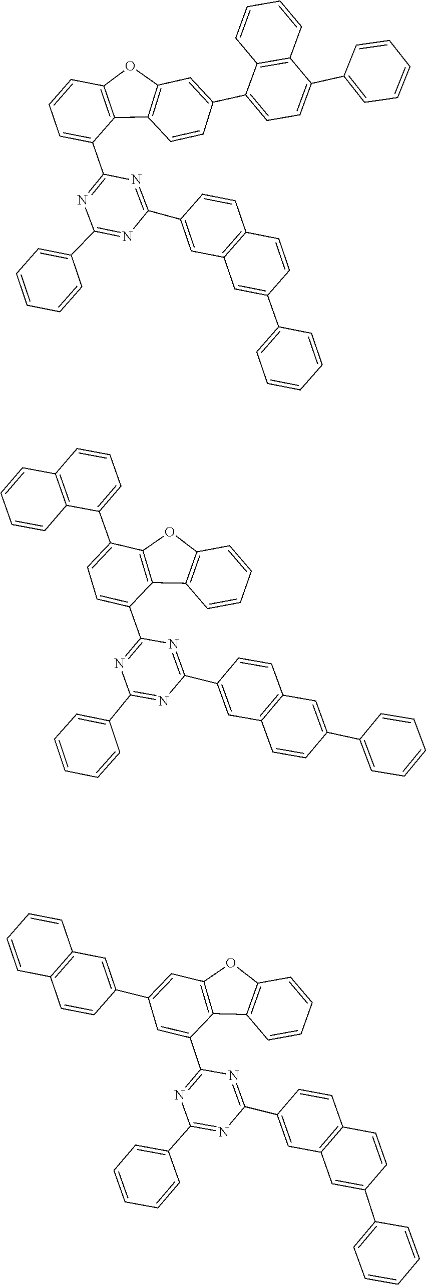 [SMILES: c1ccc(-c2ccc3ccc(-c4nc(-c5ccccc5)nc(-c5cccc6oc7cc(-c8ccc(-c9ccccc9)c9ccccc89)ccc7c56)n4)cc3c2)cc1 ; c1ccc(-c2ccc3cc(-c4nc(-c5ccccc5)nc(-c5ccc(-c6cccc7ccccc67)c6oc7ccccc7c56)n4)ccc3c2)cc1 ; c1ccc(-c2ccc3ccc(-c4nc(-c5ccccc5)nc(-c5cc(-c6ccc7ccccc7c6)cc6oc7ccccc7c56)n4)cc3c2)cc1]

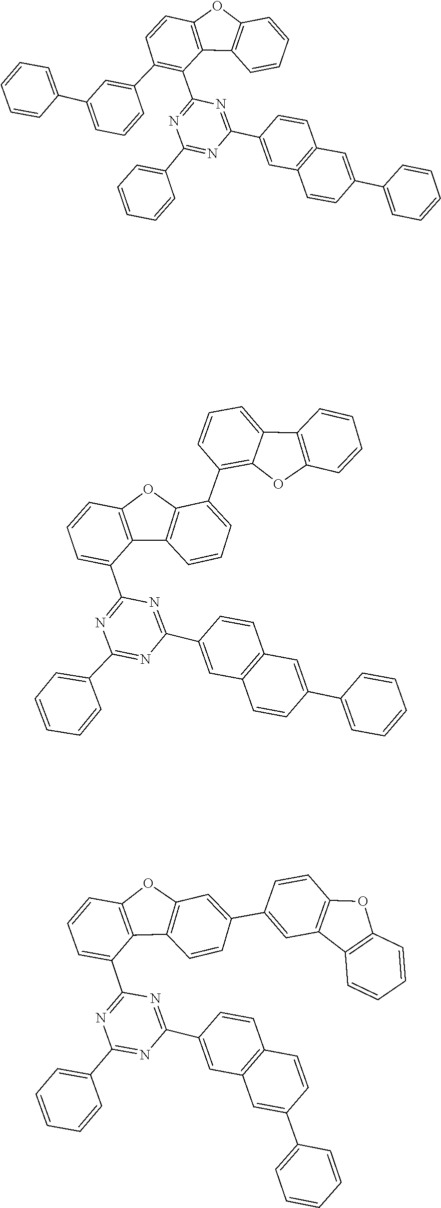 [SMILES: c1ccc(-c2cccc(-c3ccc4oc5ccccc5c4c3-c3nc(-c4ccccc4)nc(-c4ccc5cc(-c6ccccc6)ccc5c4)n3)c2)cc1 ; c1ccc(-c2ccc3cc(-c4nc(-c5ccccc5)nc(-c5cccc6oc7c(-c8cccc9c8oc8ccccc89)cccc7c56)n4)ccc3c2)cc1 ; c1ccc(-c2ccc3ccc(-c4nc(-c5ccccc5)nc(-c5cccc6oc7cc(-c8ccc9oc%10ccccc%10c9c8)ccc7c56)n4)cc3c2)cc1]

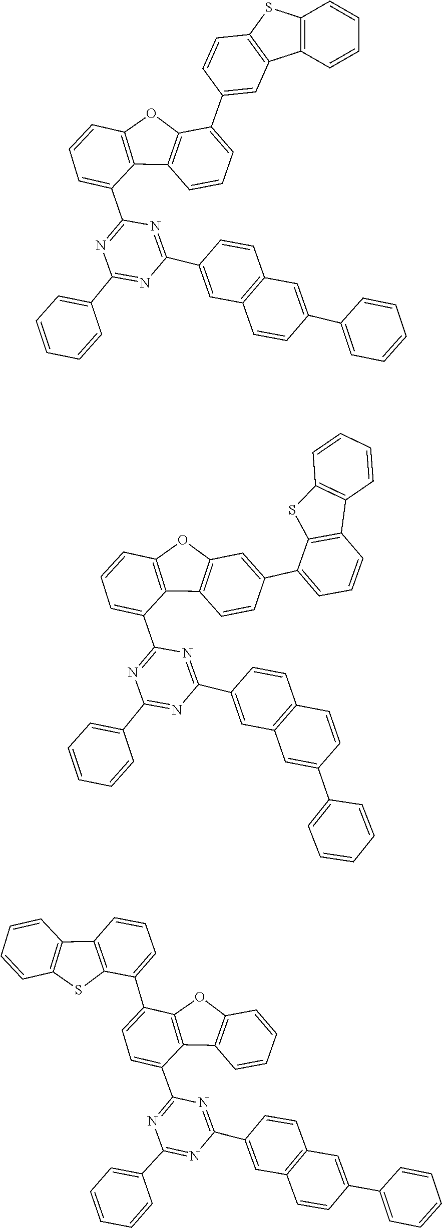 [SMILES: c1ccc(-c2ccc3cc(-c4nc(-c5ccccc5)nc(-c5cccc6oc7c(-c8ccc9sc%10ccccc%10c9c8)cccc7c56)n4)ccc3c2)cc1 ; c1ccc(-c2ccc3ccc(-c4nc(-c5ccccc5)nc(-c5cccc6oc7cc(-c8cccc9c8sc8ccccc89)ccc7c56)n4)cc3c2)cc1 ; c1ccc(-c2ccc3cc(-c4nc(-c5ccccc5)nc(-c5ccc(-c6cccc7c6sc6ccccc67)c6oc7ccccc7c56)n4)ccc3c2)cc1]

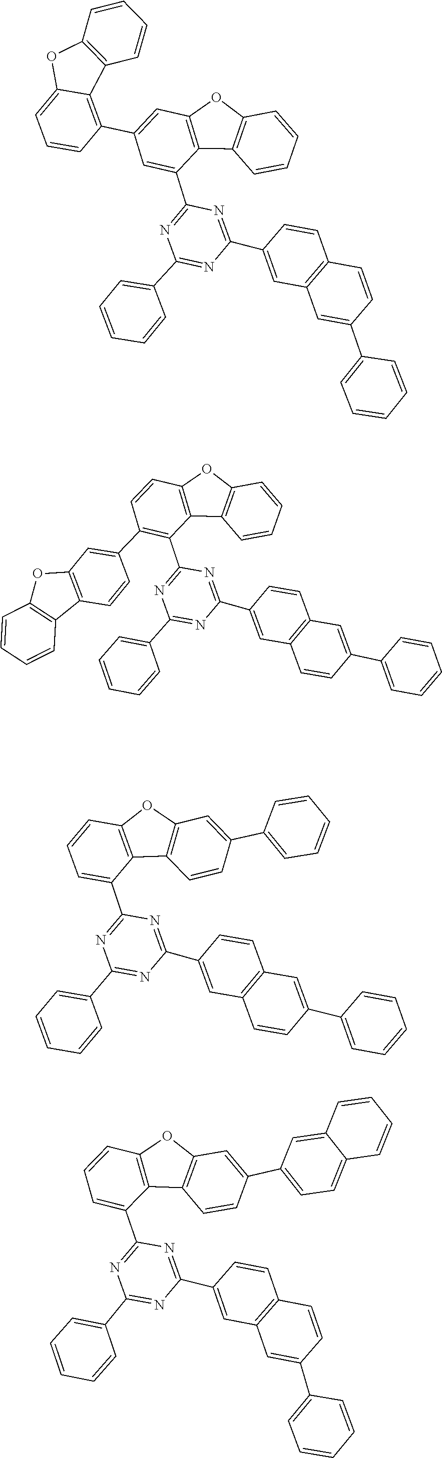

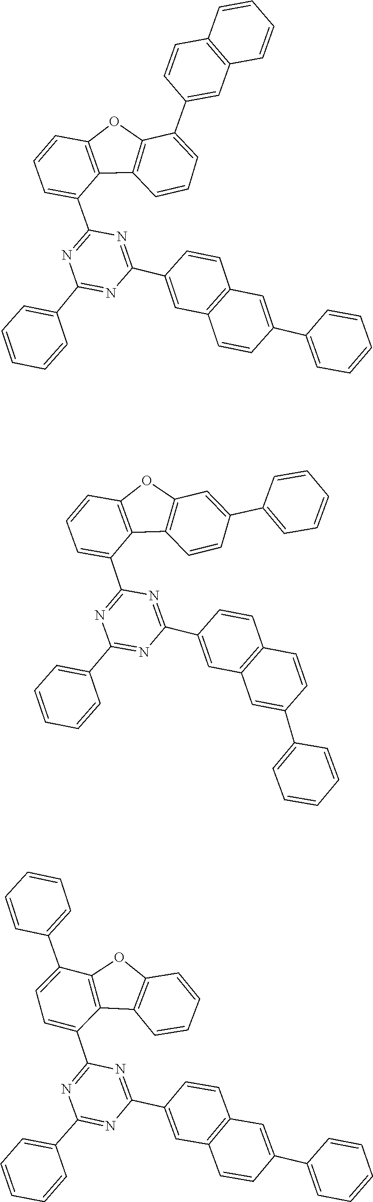 [SMILES: c1ccc(-c2ccc3cc(-c4nc(-c5ccccc5)nc(-c5cccc6oc7c(-c8ccc9ccccc9c8)cccc7c56)n4)ccc3c2)cc1 ; c1ccc(-c2ccc3ccc(-c4nc(-c5ccccc5)nc(-c5cccc6oc7cc(-c8ccccc8)ccc7c56)n4)cc3c2)cc1 ; c1ccc(-c2ccc3cc(-c4nc(-c5ccccc5)nc(-c5ccc(-c6ccccc6)c6oc7ccccc7c56)n4)ccc3c2)cc1]

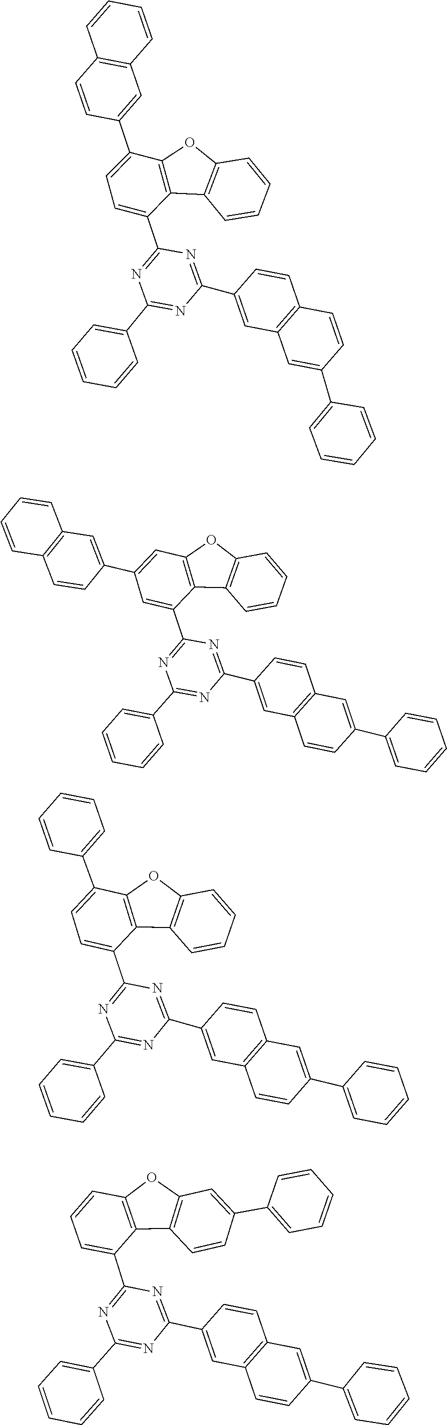

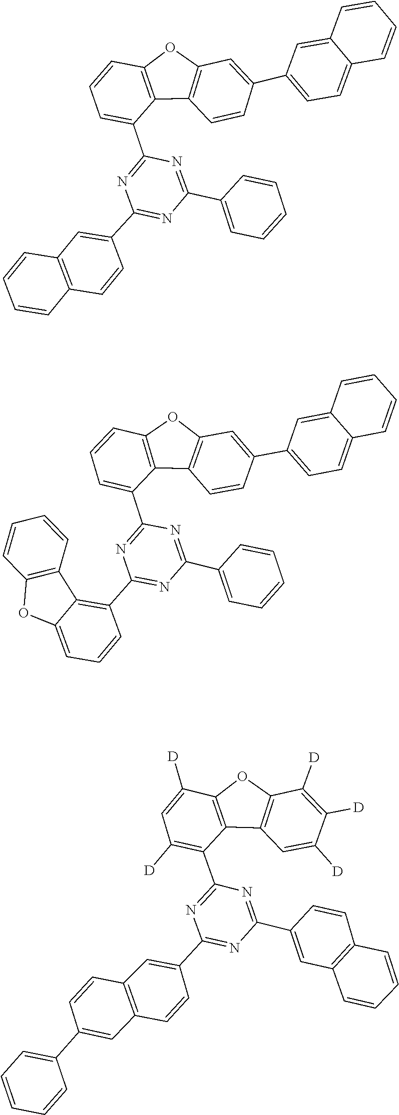 [SMILES: c1ccc(-c2nc(-c3ccc4ccccc4c3)nc(-c3cccc4oc5cc(-c6ccc7ccccc7c6)ccc5c34)n2)cc1 ; c1ccc(-c2nc(-c3cccc4oc5ccccc5c34)nc(-c3cccc4oc5cc(-c6ccc7ccccc7c6)ccc5c34)n2)cc1 ; [2H]c1cc2c(oc3c([2H])cc([2H])c(-c4nc(-c5ccc6ccccc6c5)nc(-c5ccc6cc(-c7ccccc7)ccc6c5)n4)c32)c([2H])c1[2H]]

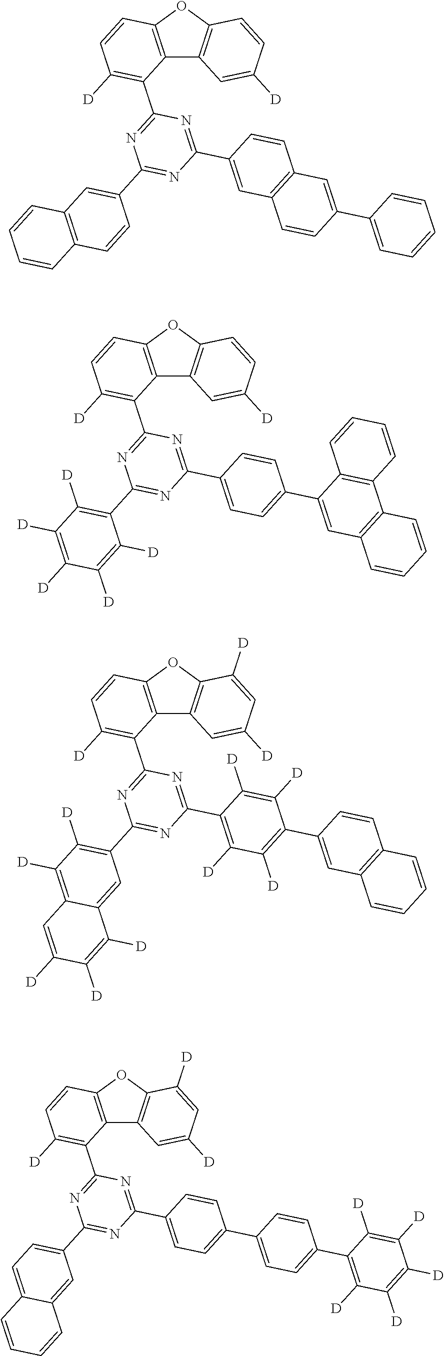 [SMILES: [2H]c1ccc2oc3ccc([2H])c(-c4nc(-c5ccc6ccccc6c5)nc(-c5ccc6cc(-c7ccccc7)ccc6c5)n4)c3c2c1 ; [2H]c1ccc2oc3ccc([2H])c(-c4nc(-c5ccc(-c6cc7ccccc7c7ccccc67)cc5)nc(-c5c([2H])c([2H])c([2H])c([2H])c5[2H])n4)c3c2c1 ; [2H]c1cc([2H])c2oc3ccc([2H])c(-c4nc(-c5cc6c([2H])c([2H])c([2H])cc6c([2H])c5[2H])nc(-c5c([2H])c([2H])c(-c6ccc7ccccc7c6)c([2H])c5[2H])n4)c3c2c1 ; [2H]c1cc([2H])c2oc3ccc([2H])c(-c4nc(-c5ccc(-c6ccc(-c7c([2H])c([2H])c([2H])c([2H])c7[2H])cc6)cc5)nc(-c5ccc6ccccc6c5)n4)c3c2c1]

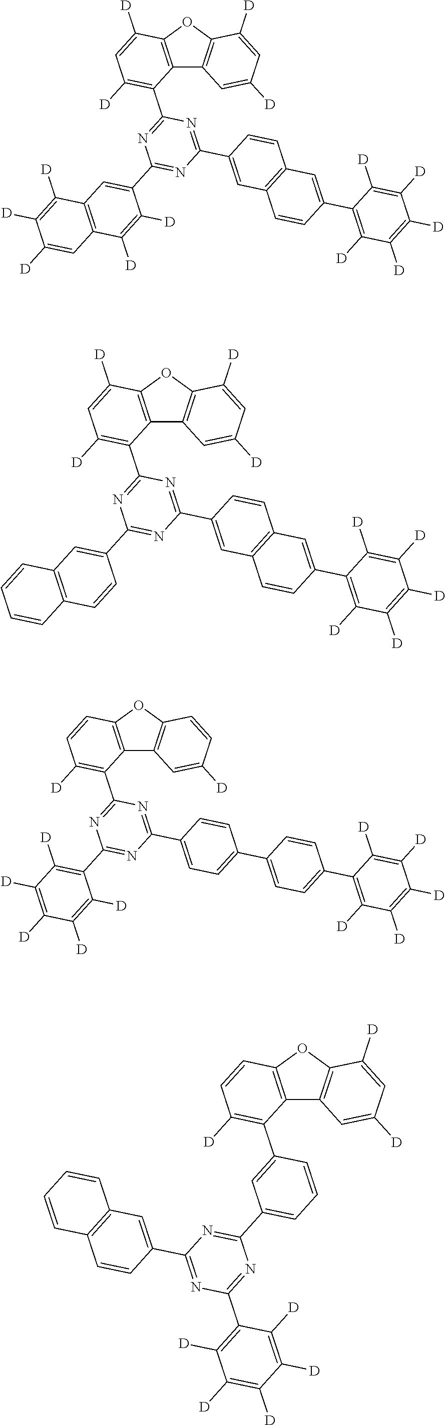

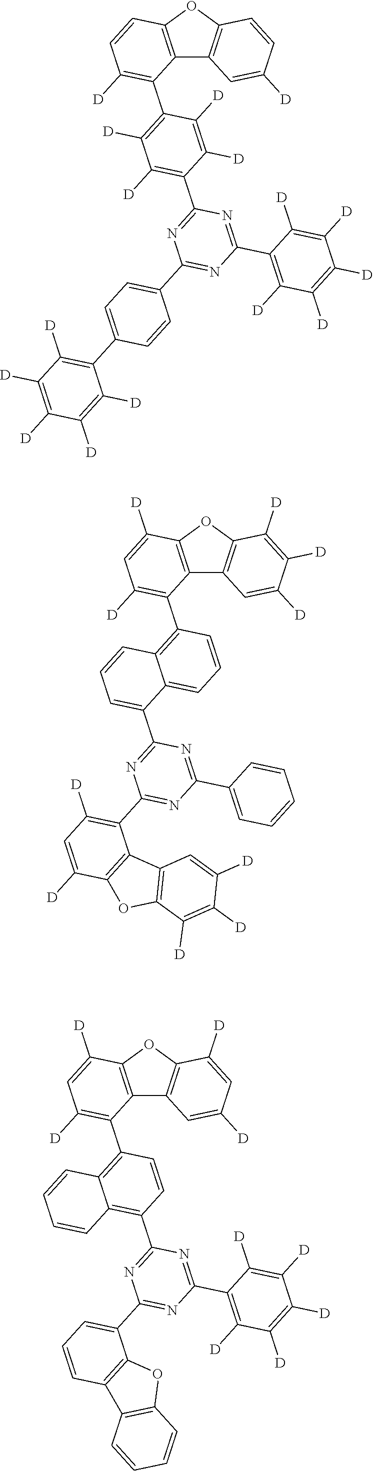 [SMILES: [2H]c1ccc2oc3ccc([2H])c(-c4c([2H])c([2H])c(-c5nc(-c6ccc(-c7c([2H])c([2H])c([2H])c([2H])c7[2H])cc6)nc(-c6c([2H])c([2H])c([2H])c([2H])c6[2H])n5)c([2H])c4[2H])c3c2c1 ; [2H]c1cc2c(oc3c([2H])cc([2H])c(-c4nc(-c5ccccc5)nc(-c5cccc6c(-c7c([2H])cc([2H])c8oc9c([2H])c([2H])c([2H])cc9c78)cccc56)n4)c32)c([2H])c1[2H] ; [2H]c1cc([2H])c2oc3c([2H])cc([2H])c(-c4ccc(-c5nc(-c6c([2H])c([2H])c([2H])c([2H])c6[2H])nc(-c6cccc7c6oc6ccccc67)n5)c5ccccc45)c3c2c1]

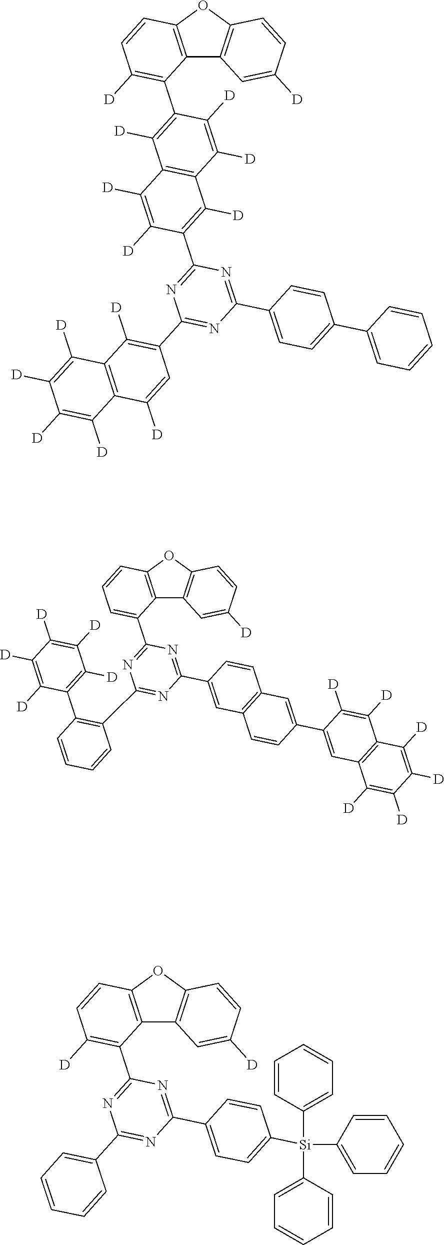 [SMILES: [2H]c1ccc2oc3ccc([2H])c(-c4c([2H])c([2H])c5c([2H])c(-c6nc(-c7ccc(-c8ccccc8)cc7)nc(-c7cc([2H])c8c([2H])c([2H])c([2H])c([2H])c8c7[2H])n6)c([2H])c([2H])c5c4[2H])c3c2c1 ; [2H]c1ccc2oc3cccc(-c4nc(-c5ccc6cc(-c7cc8c([2H])c([2H])c([2H])c([2H])c8c([2H])c7[2H])ccc6c5)nc(-c5ccccc5-c5c([2H])c([2H])c([2H])c([2H])c5[2H])n4)c3c2c1 ; [2H]c1ccc2oc3ccc([2H])c(-c4nc(-c5ccccc5)nc(-c5ccc([Si](c6ccccc6)(c6ccccc6)c6ccccc6)cc5)n4)c3c2c1]

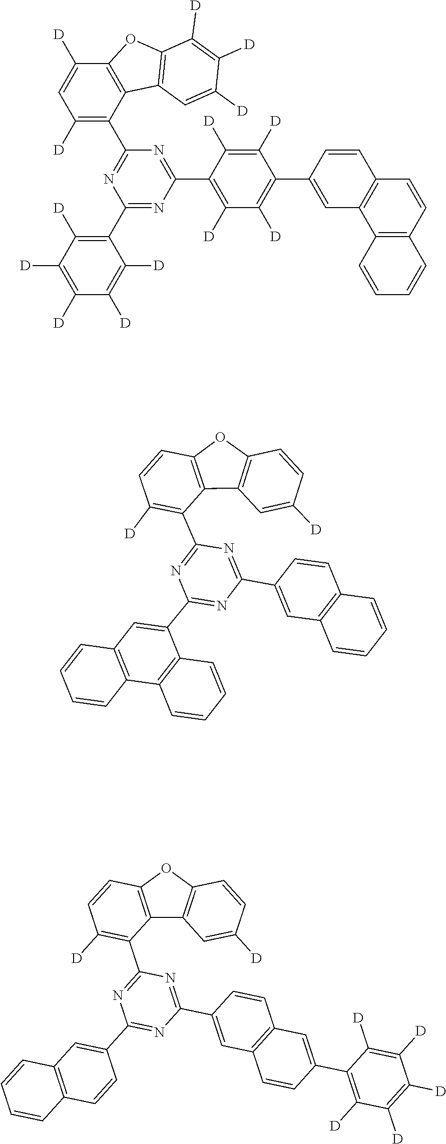 [SMILES: [2H]c1cc2c(oc3c([2H])cc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c32)c([2H])c1[2H] ; [2H]c1ccc2oc3ccc([2H])c(-c4nc(-c5ccc6ccccc6c5)nc(-c5cc6ccccc6c6ccccc56)n4)c3c2c1 ; [2H]c1ccc2oc3ccc([2H])c(-c4nc(-c5ccc6ccccc6c5)nc(-c5ccc6cc(-c7c([2H])c([2H])c([2H])c([2H])c7[2H])ccc6c5)n4)c3c2c1]

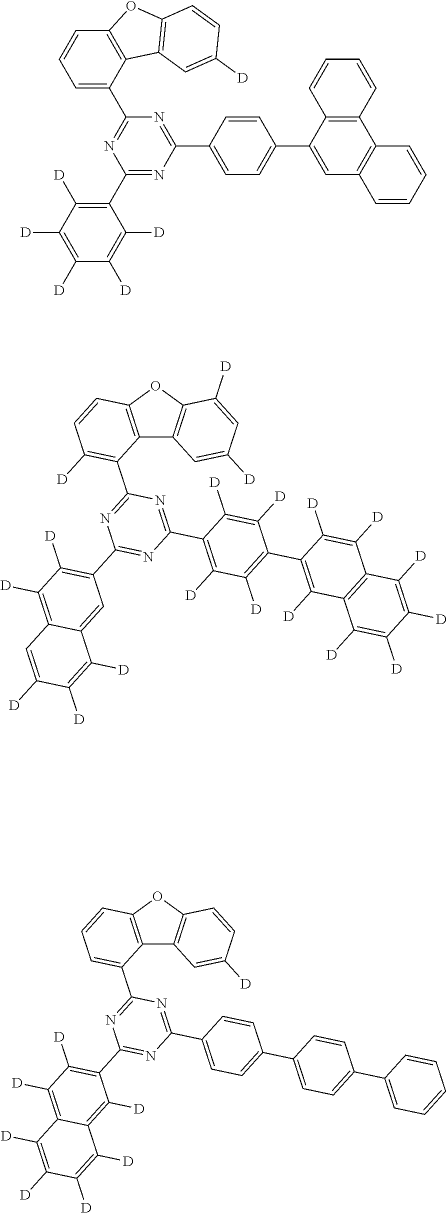 [SMILES: [2H]c1ccc2oc3cccc(-c4nc(-c5ccc(-c6cc7ccccc7c7ccccc67)cc5)nc(-c5c([2H])c([2H])c([2H])c([2H])c5[2H])n4)c3c2c1 ; [2H]c1cc([2H])c2oc3ccc([2H])c(-c4nc(-c5cc6c([2H])c([2H])c([2H])cc6c([2H])c5[2H])nc(-c5c([2H])c([2H])c(-c6c([2H])c([2H])c7c([2H])c([2H])c([2H])c([2H])c7c6[2H])c([2H])c5[2H])n4)c3c2c1 ; [2H]c1ccc2oc3cccc(-c4nc(-c5ccc(-c6ccc(-c7ccccc7)cc6)cc5)nc(-c5c([2H])c([2H])c6c([2H])c([2H])c([2H])c([2H])c6c5[2H])n4)c3c2c1]

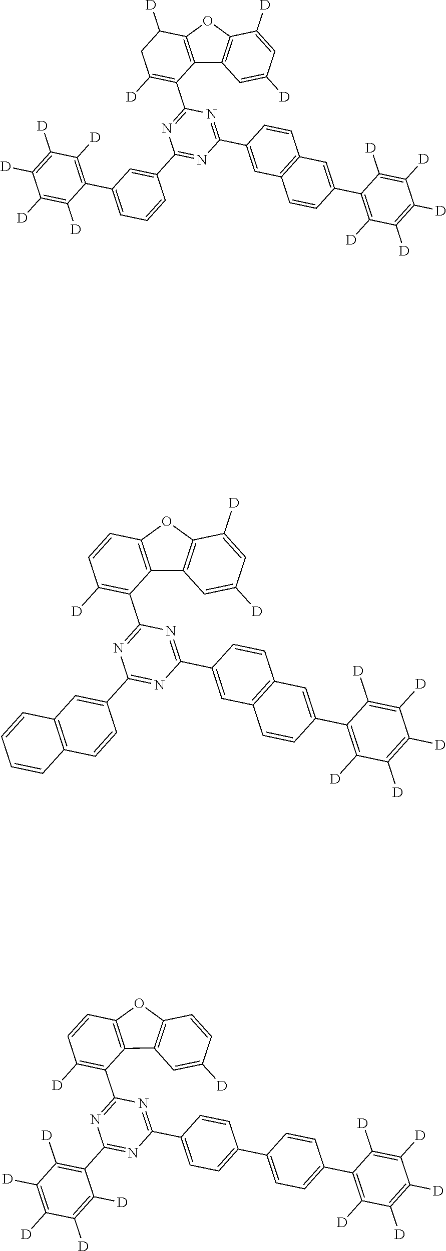 [SMILES: [2H]C1=C(c2nc(-c3cccc(-c4c([2H])c([2H])c([2H])c([2H])c4[2H])c3)nc(-c3ccc4cc(-c5c([2H])c([2H])c([2H])c([2H])c5[2H])ccc4c3)n2)c2c(oc3c([2H])cc([2H])cc23)C([2H])C1 ; [2H]c1cc([2H])c2oc3ccc([2H])c(-c4nc(-c5ccc6ccccc6c5)nc(-c5ccc6cc(-c7c([2H])c([2H])c([2H])c([2H])c7[2H])ccc6c5)n4)c3c2c1 ; [2H]c1ccc2oc3ccc([2H])c(-c4nc(-c5ccc(-c6ccc(-c7c([2H])c([2H])c([2H])c([2H])c7[2H])cc6)cc5)nc(-c5c([2H])c([2H])c([2H])c([2H])c5[2H])n4)c3c2c1]

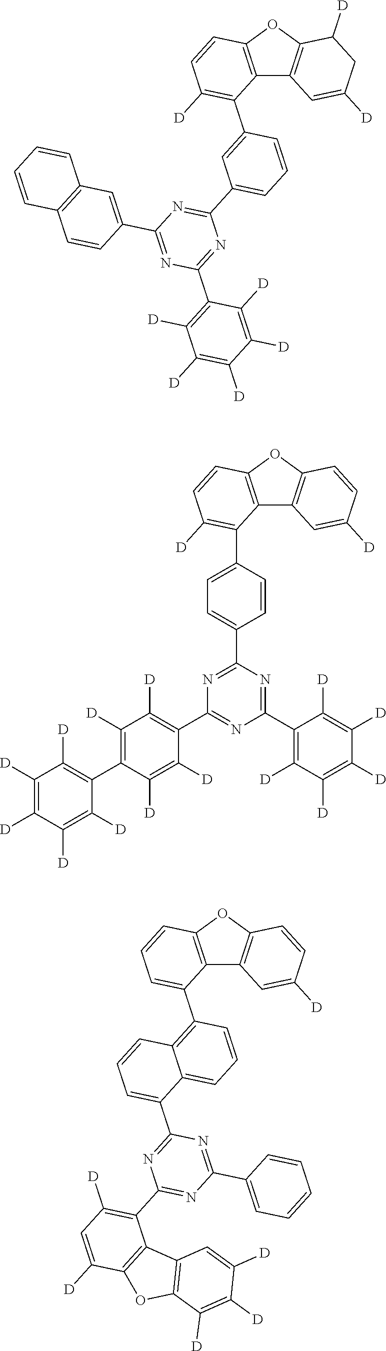 [SMILES: [2H]C1=Cc2c(oc3ccc([2H])c(-c4cccc(-c5nc(-c6ccc7ccccc7c6)nc(-c6c([2H])c([2H])c([2H])c([2H])c6[2H])n5)c4)c23)C([2H])C1 ; [2H]c1ccc2oc3ccc([2H])c(-c4ccc(-c5nc(-c6c([2H])c([2H])c([2H])c([2H])c6[2H])nc(-c6c([2H])c([2H])c(-c7c([2H])c([2H])c([2H])c([2H])c7[2H])c([2H])c6[2H])n5)cc4)c3c2c1 ; [2H]c1ccc2oc3cccc(-c4cccc5c(-c6nc(-c7ccccc7)nc(-c7c([2H])cc([2H])c8oc9c([2H])c([2H])c([2H])cc9c78)n6)cccc45)c3c2c1]

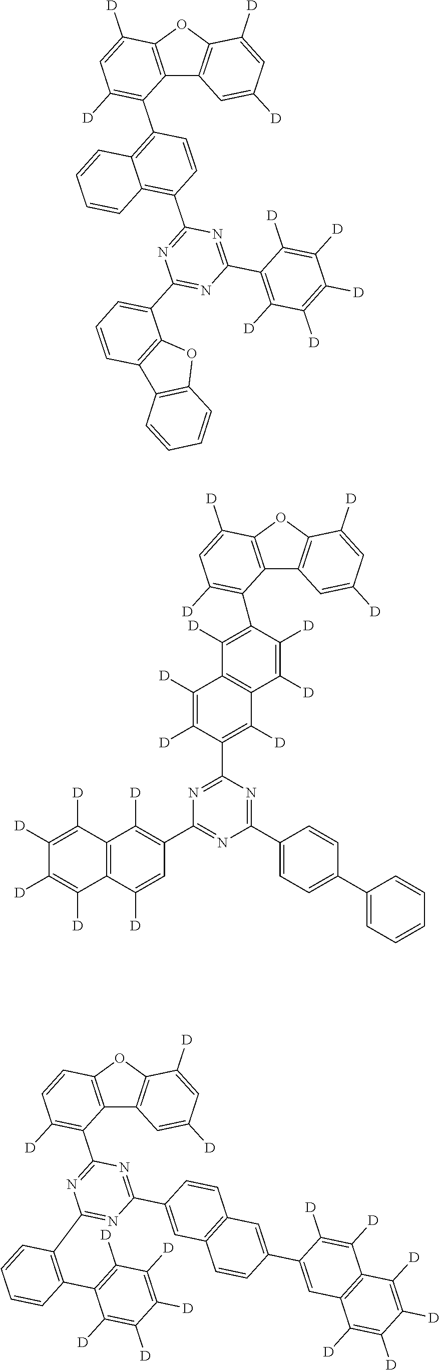 [SMILES: [2H]c1cc([2H])c2oc3c([2H])cc([2H])c(-c4ccc(-c5nc(-c6c([2H])c([2H])c([2H])c([2H])c6[2H])nc(-c6cccc7c6oc6ccccc67)n5)c5ccccc45)c3c2c1 ; [2H]c1cc([2H])c2oc3c([2H])cc([2H])c(-c4c([2H])c([2H])c5c([2H])c(-c6nc(-c7ccc(-c8ccccc8)cc7)nc(-c7cc([2H])c8c([2H])c([2H])c([2H])c([2H])c8c7[2H])n6)c([2H])c([2H])c5c4[2H])c3c2c1 ; [2H]c1cc([2H])c2oc3ccc([2H])c(-c4nc(-c5ccc6cc(-c7cc8c([2H])c([2H])c([2H])c([2H])c8c([2H])c7[2H])ccc6c5)nc(-c5ccccc5-c5c([2H])c([2H])c([2H])c([2H])c5[2H])n4)c3c2c1]

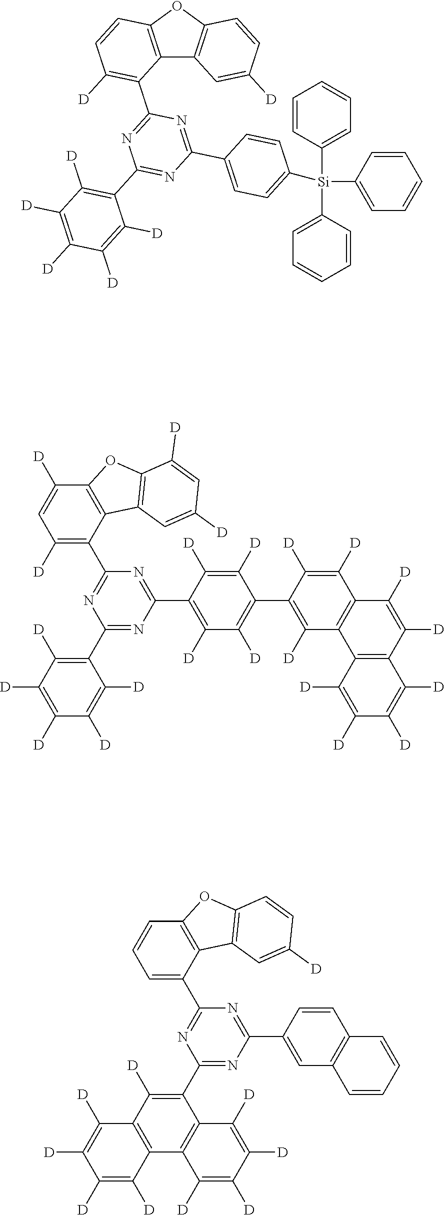 [SMILES: [2H]c1ccc2oc3ccc([2H])c(-c4nc(-c5ccc([Si](c6ccccc6)(c6ccccc6)c6ccccc6)cc5)nc(-c5c([2H])c([2H])c([2H])c([2H])c5[2H])n4)c3c2c1 ; [2H]c1cc([2H])c2oc3c([2H])cc([2H])c(-c4nc(-c5c([2H])c([2H])c([2H])c([2H])c5[2H])nc(-c5c([2H])c([2H])c(-c6c([2H])c([2H])c7c([2H])c([2H])c8c([2H])c([2H])c([2H])c([2H])c8c7c6[2H])c([2H])c5[2H])n4)c3c2c1 ; [2H]c1ccc2oc3cccc(-c4nc(-c5ccc6ccccc6c5)nc(-c5c([2H])c6c([2H])c([2H])c([2H])c([2H])c6c6c([2H])c([2H])c([2H])c([2H])c56)n4)c3c2c1]

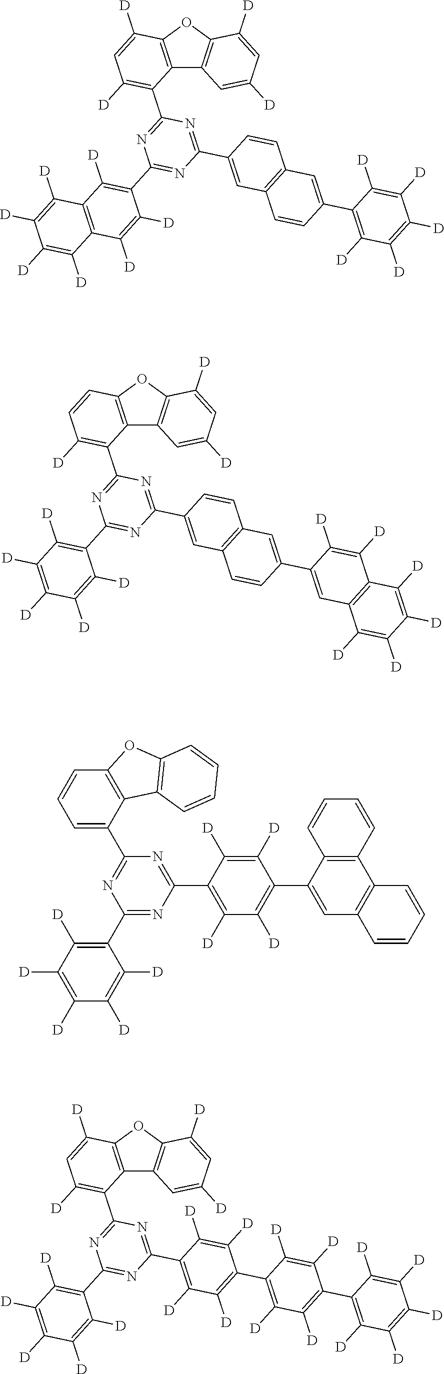

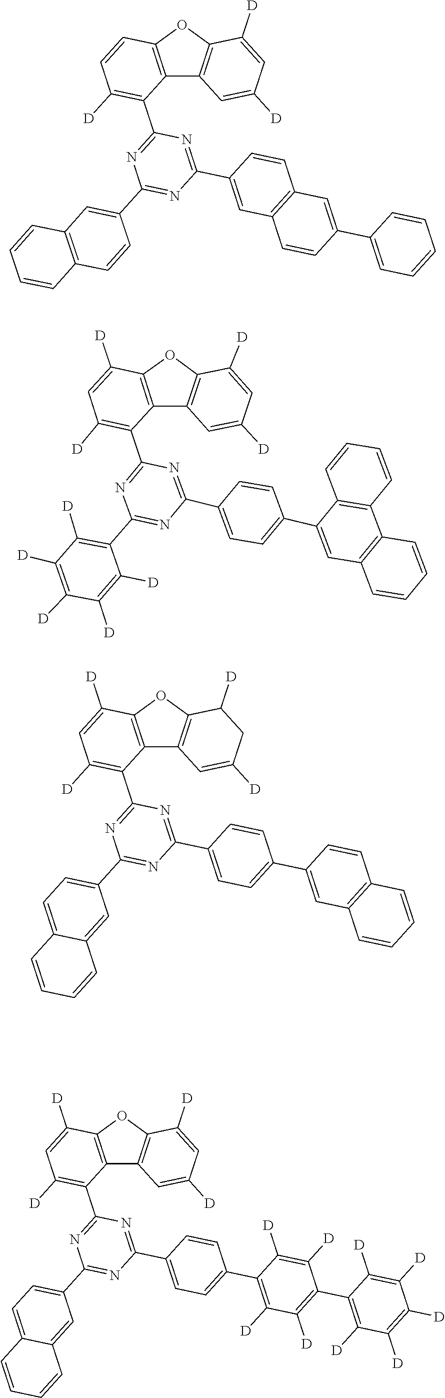 [SMILES: [2H]c1cc([2H])c2oc3ccc([2H])c(-c4nc(-c5ccc6ccccc6c5)nc(-c5ccc6cc(-c7ccccc7)ccc6c5)n4)c3c2c1 ; [2H]c1cc([2H])c2oc3c([2H])cc([2H])c(-c4nc(-c5ccc(-c6cc7ccccc7c7ccccc67)cc5)nc(-c5c([2H])c([2H])c([2H])c([2H])c5[2H])n4)c3c2c1 ; [2H]C1=Cc2c(oc3c([2H])cc([2H])c(-c4nc(-c5ccc(-c6ccc7ccccc7c6)cc5)nc(-c5ccc6ccccc6c5)n4)c23)C([2H])C1 ; [2H]c1cc([2H])c2oc3c([2H])cc([2H])c(-c4nc(-c5ccc(-c6c([2H])c([2H])c(-c7c([2H])c([2H])c([2H])c([2H])c7[2H])c([2H])c6[2H])cc5)nc(-c5ccc6ccccc6c5)n4)c3c2c1]

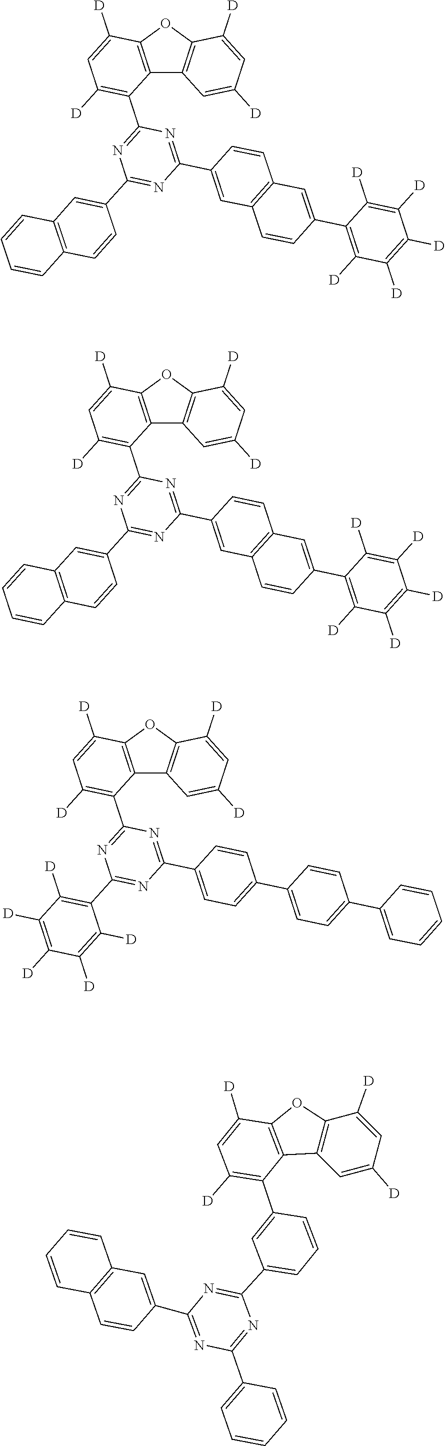 [SMILES: [2H]c1cc([2H])c2oc3c([2H])cc([2H])c(-c4nc(-c5ccc6ccccc6c5)nc(-c5ccc6cc(-c7c([2H])c([2H])c([2H])c([2H])c7[2H])ccc6c5)n4)c3c2c1 ; [2H]c1cc([2H])c2oc3c([2H])cc([2H])c(-c4nc(-c5ccc6ccccc6c5)nc(-c5ccc6cc(-c7c([2H])c([2H])c([2H])c([2H])c7[2H])ccc6c5)n4)c3c2c1 ; [2H]c1cc([2H])c2oc3c([2H])cc([2H])c(-c4nc(-c5ccc(-c6ccc(-c7ccccc7)cc6)cc5)nc(-c5c([2H])c([2H])c([2H])c([2H])c5[2H])n4)c3c2c1 ; [2H]c1cc([2H])c2oc3c([2H])cc([2H])c(-c4cccc(-c5nc(-c6ccccc6)nc(-c6ccc7ccccc7c6)n5)c4)c3c2c1]

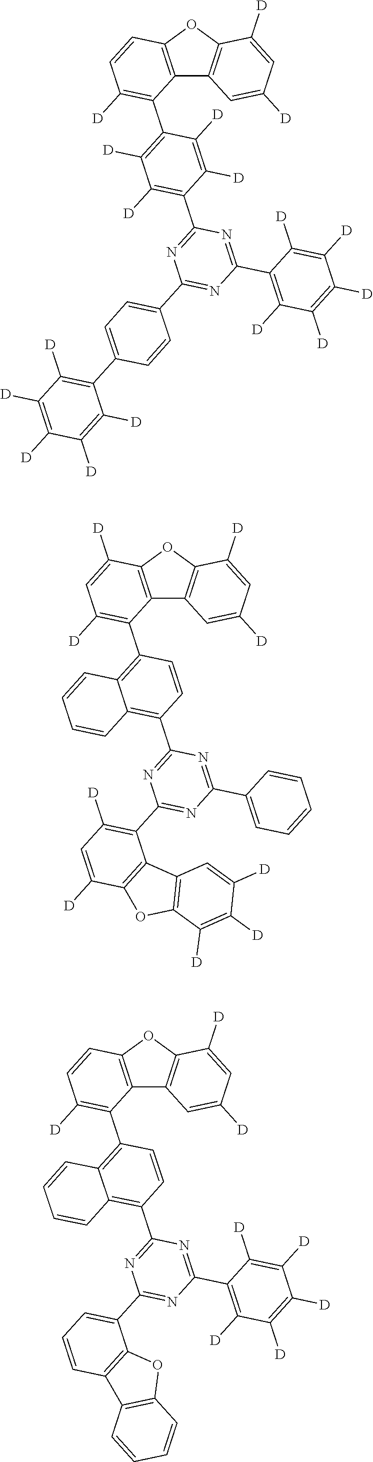 [SMILES: [2H]c1cc([2H])c2oc3ccc([2H])c(-c4c([2H])c([2H])c(-c5nc(-c6ccc(-c7c([2H])c([2H])c([2H])c([2H])c7[2H])cc6)nc(-c6c([2H])c([2H])c([2H])c([2H])c6[2H])n5)c([2H])c4[2H])c3c2c1 ; [2H]c1cc([2H])c2oc3c([2H])cc([2H])c(-c4ccc(-c5nc(-c6ccccc6)nc(-c6c([2H])cc([2H])c7oc8c([2H])c([2H])c([2H])cc8c67)n5)c5ccccc45)c3c2c1 ; [2H]c1cc([2H])c2oc3ccc([2H])c(-c4ccc(-c5nc(-c6c([2H])c([2H])c([2H])c([2H])c6[2H])nc(-c6cccc7c6oc6ccccc67)n5)c5ccccc45)c3c2c1]

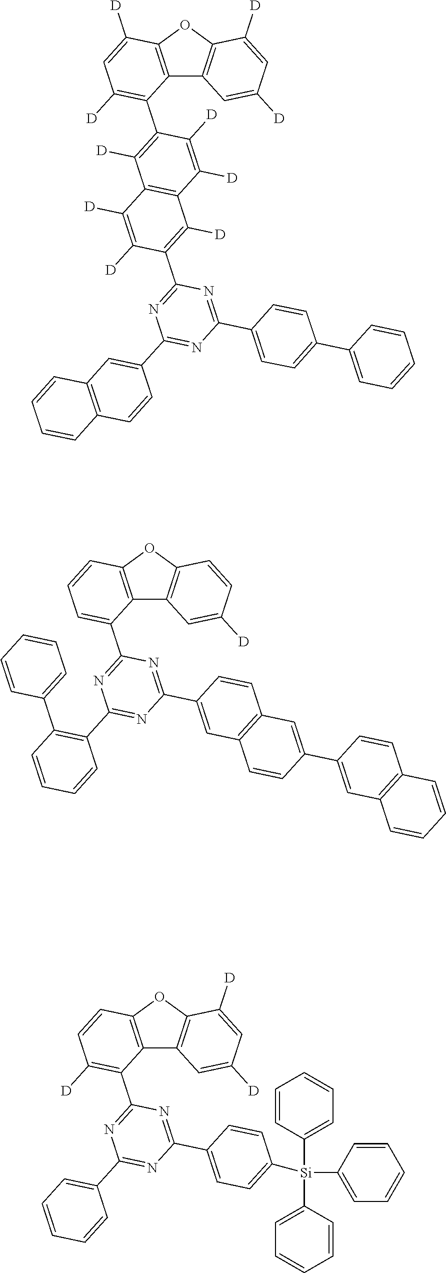 [SMILES: [2H]c1cc([2H])c2oc3c([2H])cc([2H])c(-c4c([2H])c([2H])c5c([2H])c(-c6nc(-c7ccc(-c8ccccc8)cc7)nc(-c7ccc8ccccc8c7)n6)c([2H])c([2H])c5c4[2H])c3c2c1 ; [2H]c1ccc2oc3cccc(-c4nc(-c5ccc6cc(-c7ccc8ccccc8c7)ccc6c5)nc(-c5ccccc5-c5ccccc5)n4)c3c2c1 ; [2H]c1cc([2H])c2oc3ccc([2H])c(-c4nc(-c5ccccc5)nc(-c5ccc([Si](c6ccccc6)(c6ccccc6)c6ccccc6)cc5)n4)c3c2c1]

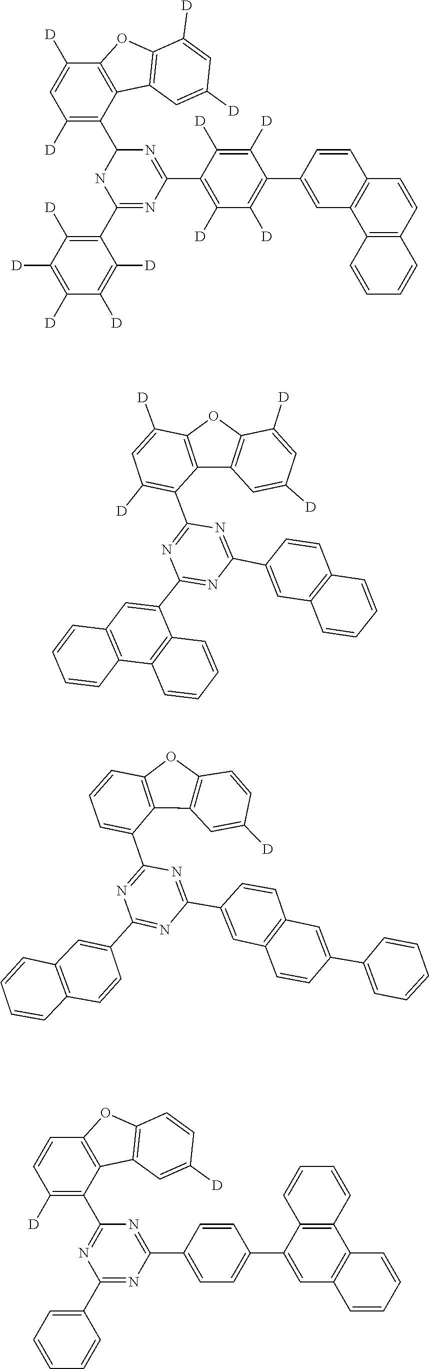 [SMILES: [2H]c1cc([2H])c2oc3c([2H])cc([2H])c(C4N=C(c5c([2H])c([2H])c(-c6ccc7ccc8ccccc8c7c6)c([2H])c5[2H])N=C(c5c([2H])c([2H])c([2H])c([2H])c5[2H])N4)c3c2c1 ; [2H]c1cc([2H])c2oc3c([2H])cc([2H])c(-c4nc(-c5ccc6ccccc6c5)nc(-c5cc6ccccc6c6ccccc56)n4)c3c2c1 ; [2H]c1ccc2oc3cccc(-c4nc(-c5ccc6ccccc6c5)nc(-c5ccc6cc(-c7ccccc7)ccc6c5)n4)c3c2c1 ; [2H]c1ccc2oc3ccc([2H])c(-c4nc(-c5ccccc5)nc(-c5ccc(-c6cc7ccccc7c7ccccc67)cc5)n4)c3c2c1]

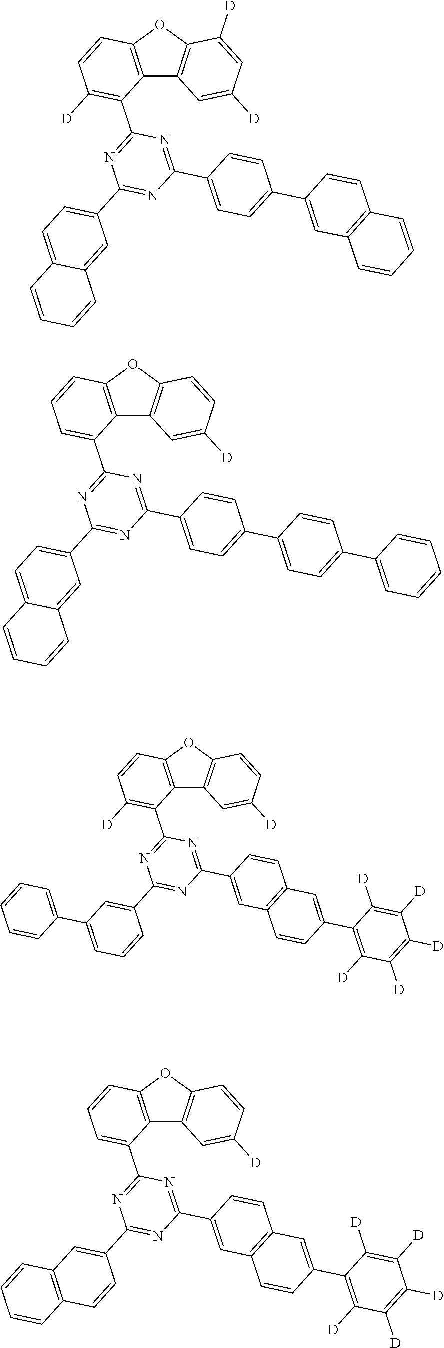 [SMILES: [2H]c1cc([2H])c2oc3ccc([2H])c(-c4nc(-c5ccc(-c6ccc7ccccc7c6)cc5)nc(-c5ccc6ccccc6c5)n4)c3c2c1 ; [2H]c1ccc2oc3cccc(-c4nc(-c5ccc(-c6ccc(-c7ccccc7)cc6)cc5)nc(-c5ccc6ccccc6c5)n4)c3c2c1 ; [2H]c1ccc2oc3ccc([2H])c(-c4nc(-c5cccc(-c6ccccc6)c5)nc(-c5ccc6cc(-c7c([2H])c([2H])c([2H])c([2H])c7[2H])ccc6c5)n4)c3c2c1 ; [2H]c1ccc2oc3cccc(-c4nc(-c5ccc6ccccc6c5)nc(-c5ccc6cc(-c7c([2H])c([2H])c([2H])c([2H])c7[2H])ccc6c5)n4)c3c2c1]

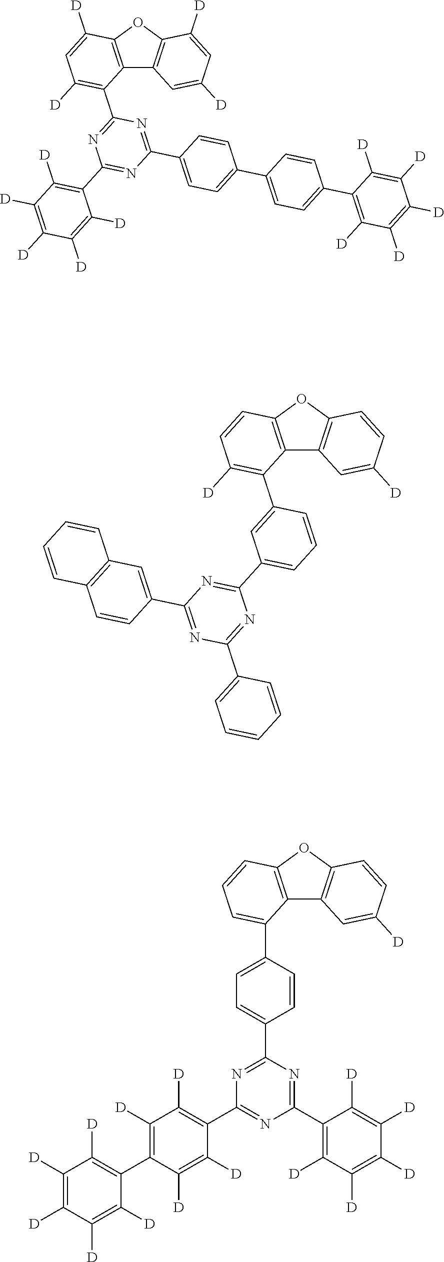 [SMILES: [2H]c1cc([2H])c2oc3c([2H])cc([2H])c(-c4nc(-c5ccc(-c6ccc(-c7c([2H])c([2H])c([2H])c([2H])c7[2H])cc6)cc5)nc(-c5c([2H])c([2H])c([2H])c([2H])c5[2H])n4)c3c2c1 ; [2H]c1ccc2oc3ccc([2H])c(-c4cccc(-c5nc(-c6ccccc6)nc(-c6ccc7ccccc7c6)n5)c4)c3c2c1 ; [2H]c1ccc2oc3cccc(-c4ccc(-c5nc(-c6c([2H])c([2H])c([2H])c([2H])c6[2H])nc(-c6c([2H])c([2H])c(-c7c([2H])c([2H])c([2H])c([2H])c7[2H])c([2H])c6[2H])n5)cc4)c3c2c1]

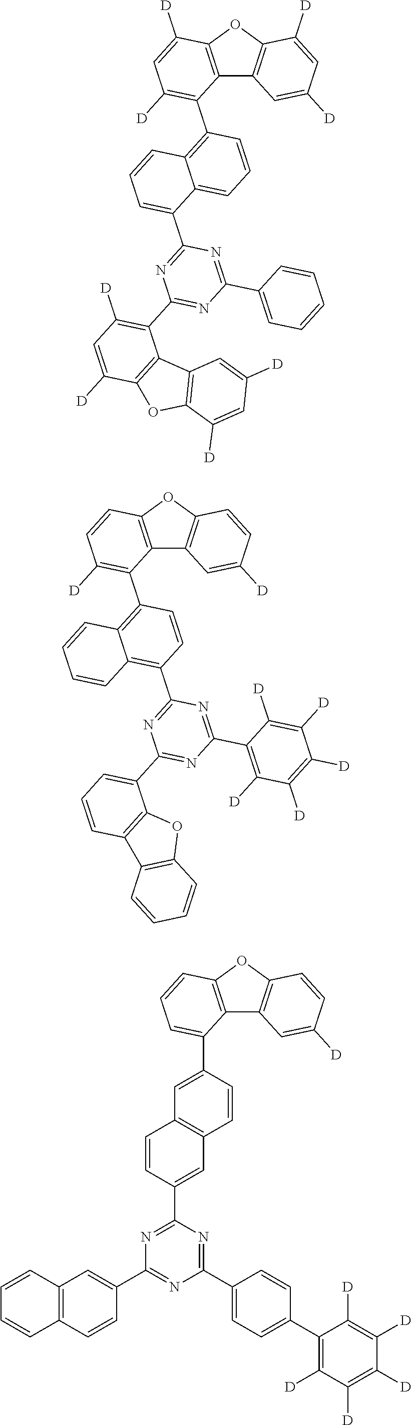 [SMILES: [2H]c1cc([2H])c2oc3c([2H])cc([2H])c(-c4nc(-c5ccccc5)nc(-c5cccc6c(-c7c([2H])cc([2H])c8oc9c([2H])cc([2H])cc9c78)cccc56)n4)c3c2c1 ; [2H]c1ccc2oc3ccc([2H])c(-c4ccc(-c5nc(-c6c([2H])c([2H])c([2H])c([2H])c6[2H])nc(-c6cccc7c6oc6ccccc67)n5)c5ccccc45)c3c2c1 ; [2H]c1ccc2oc3cccc(-c4ccc5cc(-c6nc(-c7ccc(-c8c([2H])c([2H])c([2H])c([2H])c8[2H])cc7)nc(-c7ccc8ccccc8c7)n6)ccc5c4)c3c2c1]

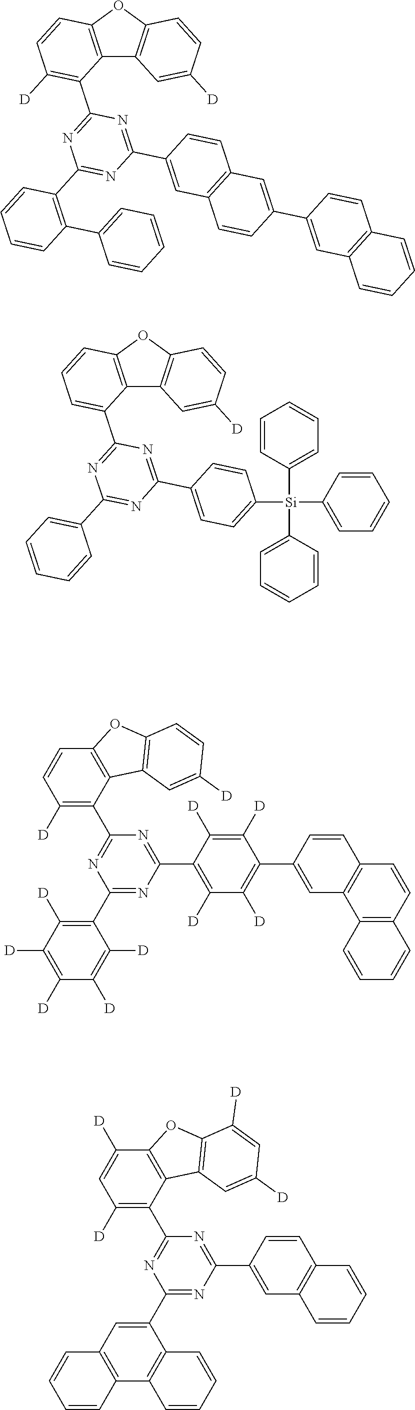 [SMILES: [2H]c1ccc2oc3ccc([2H])c(-c4nc(-c5ccc6cc(-c7ccc8ccccc8c7)ccc6c5)nc(-c5ccccc5-c5ccccc5)n4)c3c2c1 ; [2H]c1ccc2oc3cccc(-c4nc(-c5ccccc5)nc(-c5ccc([Si](c6ccccc6)(c6ccccc6)c6ccccc6)cc5)n4)c3c2c1 ; [2H]c1ccc2oc3ccc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c3c2c1 ; [2H]c1cc([2H])c2oc3c([2H])cc([2H])c(-c4nc(-c5ccc6ccccc6c5)nc(-c5cc6ccccc6c6ccccc56)n4)c3c2c1]

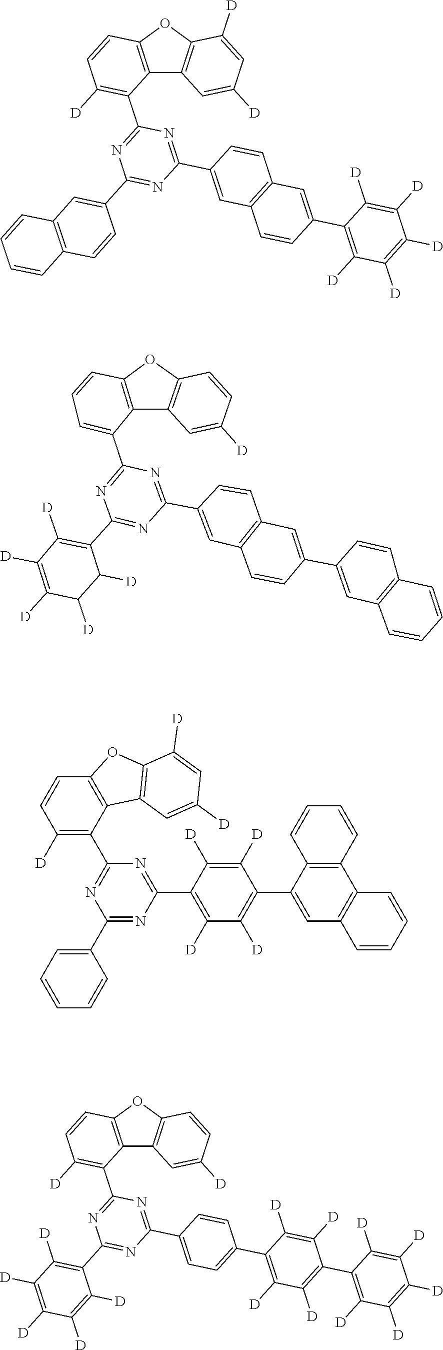

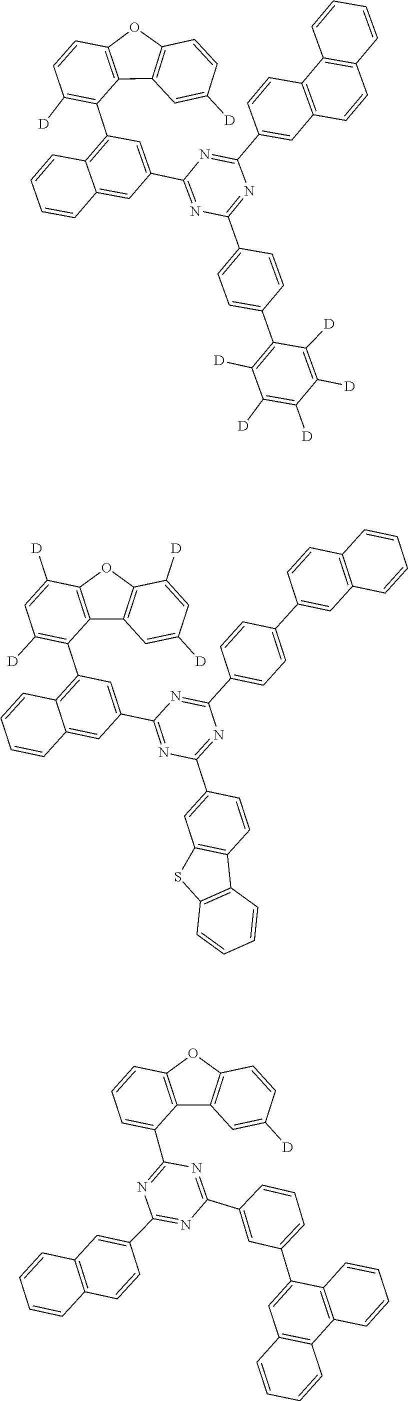 [SMILES: [2H]c1ccc2oc3ccc([2H])c(-c4cc(-c5nc(-c6ccc(-c7c([2H])c([2H])c([2H])c([2H])c7[2H])cc6)nc(-c6ccc7c(ccc8ccccc87)c6)n5)cc5ccccc45)c3c2c1 ; [2H]c1cc([2H])c2oc3c([2H])cc([2H])c(-c4cc(-c5nc(-c6ccc(-c7ccc8ccccc8c7)cc6)nc(-c6ccc7c(c6)sc6ccccc67)n5)cc5ccccc45)c3c2c1 ; [2H]c1ccc2oc3cccc(-c4nc(-c5cccc(-c6cc7ccccc7c7ccccc67)c5)nc(-c5ccc6ccccc6c5)n4)c3c2c1]

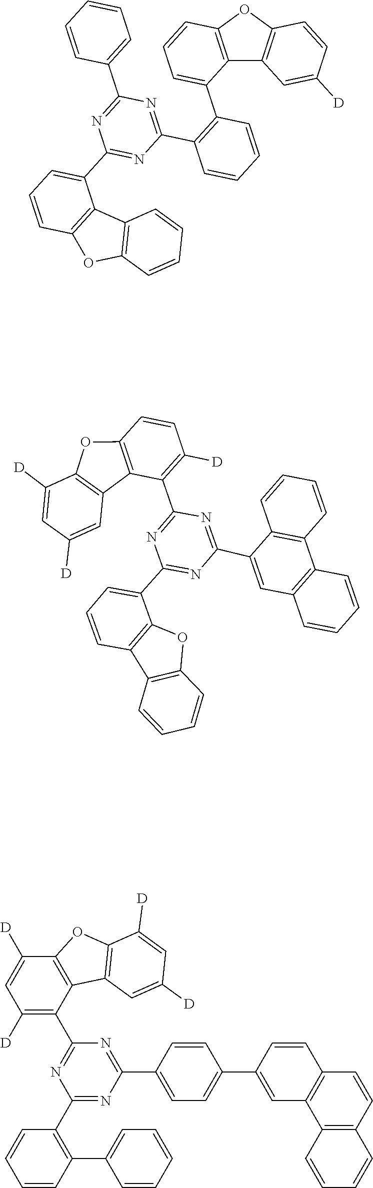 [SMILES: [2H]c1ccc2oc3cccc(-c4ccccc4-c4nc(-c5ccccc5)nc(-c5cccc6oc7ccccc7c56)n4)c3c2c1 ; [2H]c1cc([2H])c2oc3ccc([2H])c(-c4nc(-c5cc6ccccc6c6ccccc56)nc(-c5cccc6c5oc5ccccc56)n4)c3c2c1 ; [2H]c1cc([2H])c2oc3c([2H])cc([2H])c(-c4nc(-c5ccc(-c6ccc7ccc8ccccc8c7c6)cc5)nc(-c5ccccc5-c5ccccc5)n4)c3c2c1]

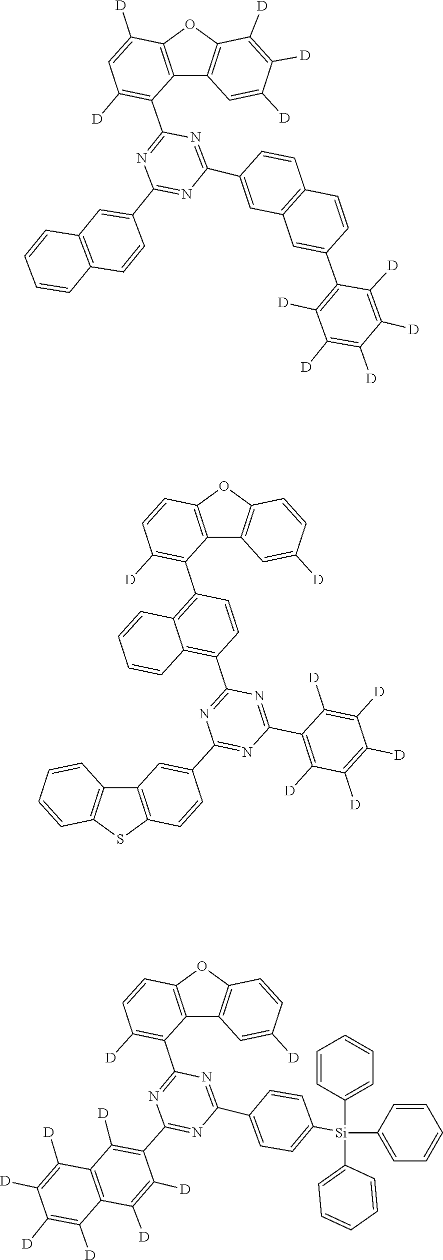 [SMILES: [2H]c1cc2c(oc3c([2H])cc([2H])c(-c4nc(-c5ccc6ccccc6c5)nc(-c5ccc6ccc(-c7c([2H])c([2H])c([2H])c([2H])c7[2H])cc6c5)n4)c32)c([2H])c1[2H] ; [2H]c1ccc2oc3ccc([2H])c(-c4ccc(-c5nc(-c6ccc7sc8ccccc8c7c6)nc(-c6c([2H])c([2H])c([2H])c([2H])c6[2H])n5)c5ccccc45)c3c2c1 ; [2H]c1ccc2oc3ccc([2H])c(-c4nc(-c5ccc([Si](c6ccccc6)(c6ccccc6)c6ccccc6)cc5)nc(-c5c([2H])c([2H])c6c([2H])c([2H])c([2H])c([2H])c6c5[2H])n4)c3c2c1]

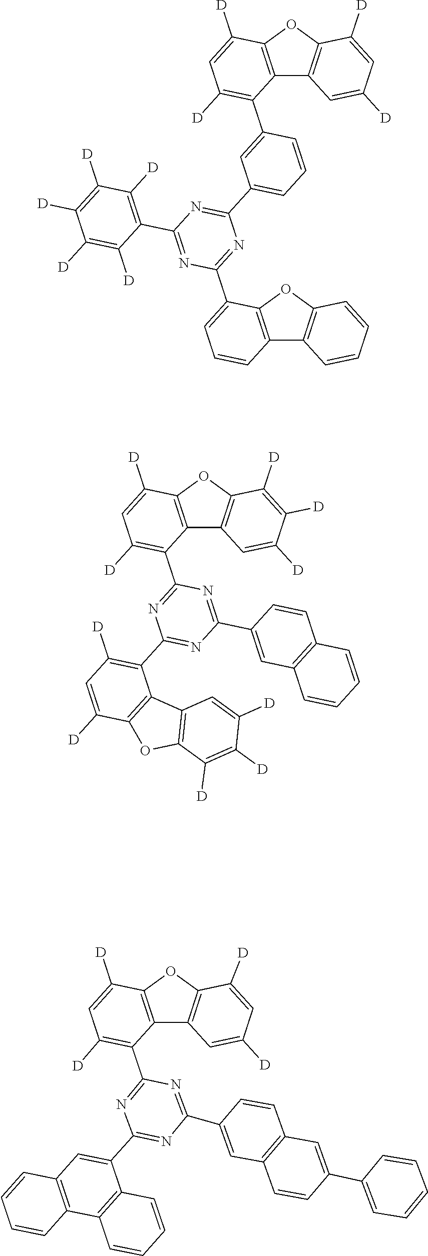 [SMILES: [2H]c1cc([2H])c2oc3c([2H])cc([2H])c(-c4cccc(-c5nc(-c6c([2H])c([2H])c([2H])c([2H])c6[2H])nc(-c6cccc7c6oc6ccccc67)n5)c4)c3c2c1 ; [2H]c1cc2c(oc3c([2H])cc([2H])c(-c4nc(-c5ccc6ccccc6c5)nc(-c5c([2H])cc([2H])c6oc7c([2H])c([2H])c([2H])cc7c56)n4)c32)c([2H])c1[2H] ; [2H]c1cc([2H])c2oc3c([2H])cc([2H])c(-c4nc(-c5ccc6cc(-c7ccccc7)ccc6c5)nc(-c5cc6ccccc6c6ccccc56)n4)c3c2c1]

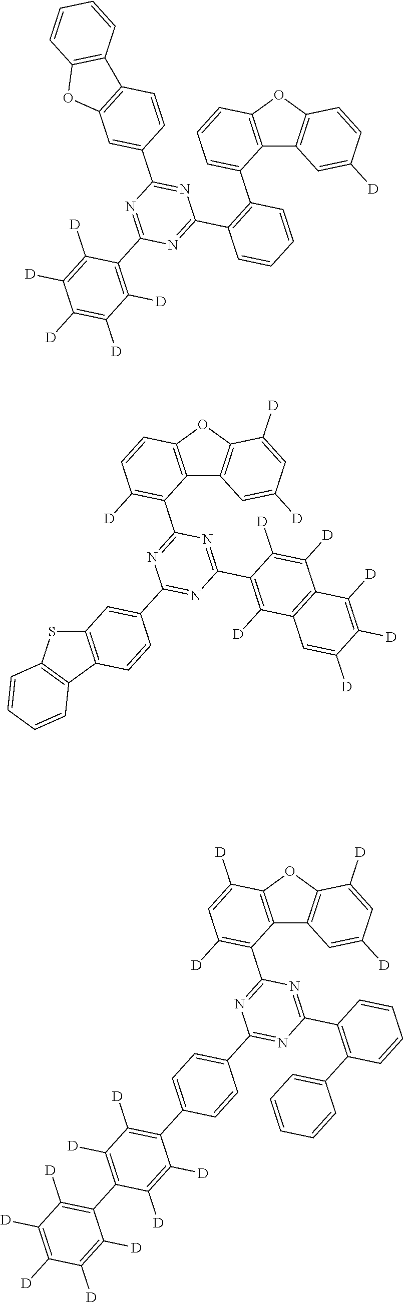 [SMILES: [2H]c1ccc2oc3cccc(-c4ccccc4-c4nc(-c5ccc6c(c5)oc5ccccc56)nc(-c5c([2H])c([2H])c([2H])c([2H])c5[2H])n4)c3c2c1 ; [2H]c1cc([2H])c2oc3ccc([2H])c(-c4nc(-c5ccc6c(c5)sc5ccccc56)nc(-c5c([2H])c([2H])c6c([2H])c([2H])c([2H])cc6c5[2H])n4)c3c2c1 ; [2H]c1cc([2H])c2oc3c([2H])cc([2H])c(-c4nc(-c5ccc(-c6c([2H])c([2H])c(-c7c([2H])c([2H])c([2H])c([2H])c7[2H])c([2H])c6[2H])cc5)nc(-c5ccccc5-c5ccccc5)n4)c3c2c1]

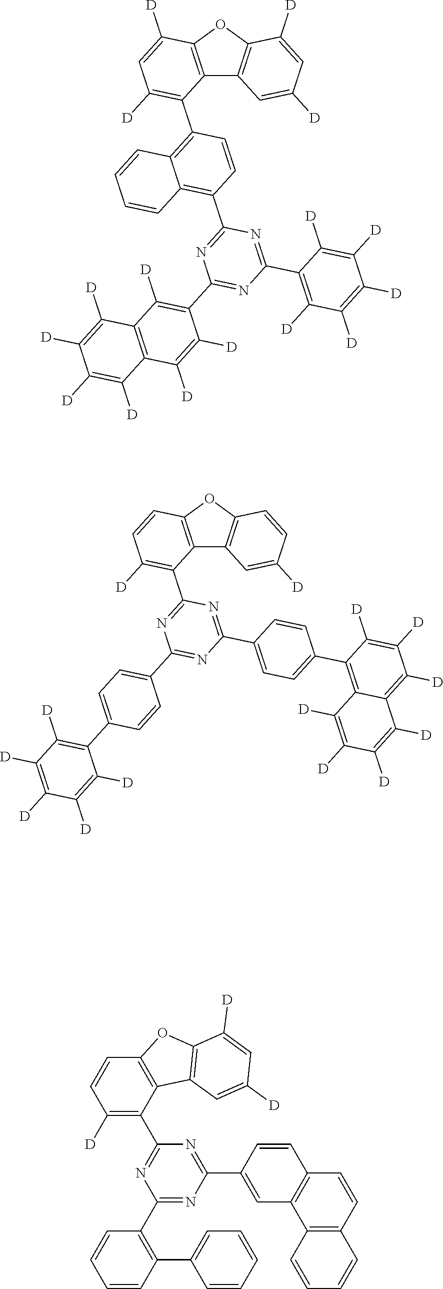 [SMILES: [2H]c1cc([2H])c2oc3c([2H])cc([2H])c(-c4ccc(-c5nc(-c6c([2H])c([2H])c([2H])c([2H])c6[2H])nc(-c6c([2H])c([2H])c7c([2H])c([2H])c([2H])c([2H])c7c6[2H])n5)c5ccccc45)c3c2c1 ; [2H]c1ccc2oc3ccc([2H])c(-c4nc(-c5ccc(-c6c([2H])c([2H])c([2H])c([2H])c6[2H])cc5)nc(-c5ccc(-c6c([2H])c([2H])c([2H])c7c([2H])c([2H])c([2H])c([2H])c67)cc5)n4)c3c2c1 ; [2H]c1cc([2H])c2oc3ccc([2H])c(-c4nc(-c5ccc6ccc7ccccc7c6c5)nc(-c5ccccc5-c5ccccc5)n4)c3c2c1]

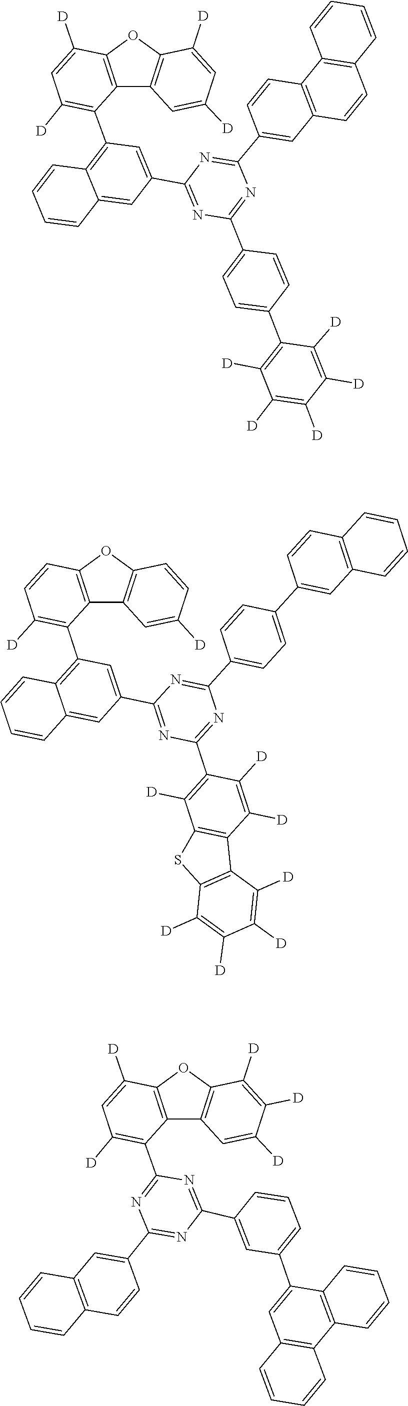 [SMILES: [2H]c1cc([2H])c2oc3c([2H])cc([2H])c(-c4cc(-c5nc(-c6ccc(-c7c([2H])c([2H])c([2H])c([2H])c7[2H])cc6)nc(-c6ccc7c(ccc8ccccc87)c6)n5)cc5ccccc45)c3c2c1 ; [2H]c1ccc2oc3ccc([2H])c(-c4cc(-c5nc(-c6ccc(-c7ccc8ccccc8c7)cc6)nc(-c6c([2H])c([2H])c7c(sc8c([2H])c([2H])c([2H])c([2H])c87)c6[2H])n5)cc5ccccc45)c3c2c1 ; [2H]c1cc2c(oc3c([2H])cc([2H])c(-c4nc(-c5cccc(-c6cc7ccccc7c7ccccc67)c5)nc(-c5ccc6ccccc6c5)n4)c32)c([2H])c1[2H]]

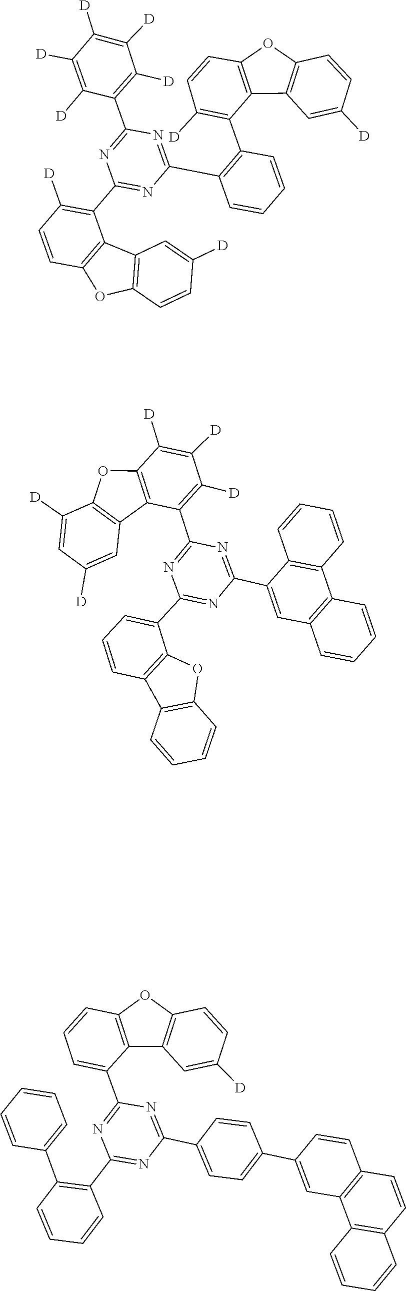 [SMILES: [2H]c1ccc2oc3ccc([2H])c(-c4nc(-c5ccccc5-c5c([2H])ccc6oc7ccc([2H])cc7c56)nc(-c5c([2H])c([2H])c([2H])c([2H])c5[2H])n4)c3c2c1 ; [2H]c1cc([2H])c2oc3c([2H])c([2H])c([2H])c(-c4nc(-c5cc6ccccc6c6ccccc56)nc(-c5cccc6c5oc5ccccc56)n4)c3c2c1 ; [2H]c1ccc2oc3cccc(-c4nc(-c5ccc(-c6ccc7ccc8ccccc8c7c6)cc5)nc(-c5ccccc5-c5ccccc5)n4)c3c2c1]

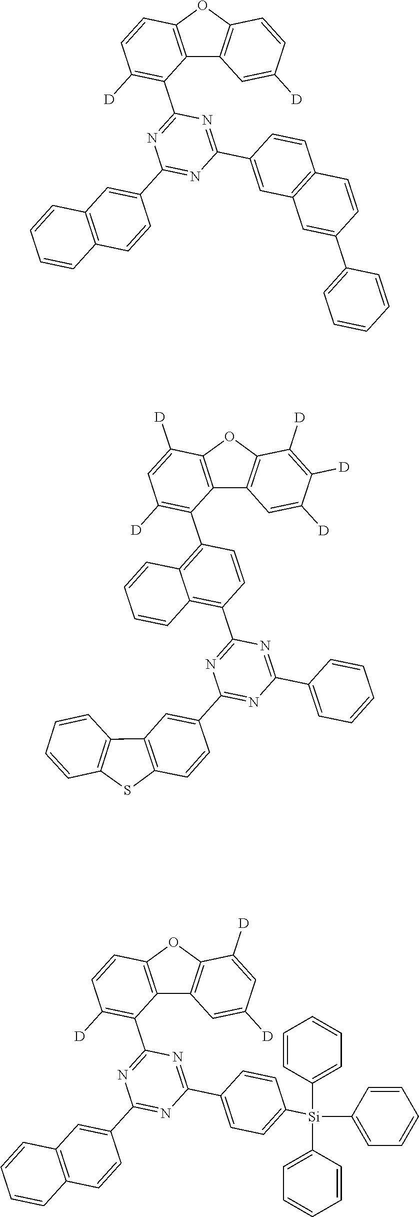 [SMILES: [2H]c1ccc2oc3ccc([2H])c(-c4nc(-c5ccc6ccccc6c5)nc(-c5ccc6ccc(-c7ccccc7)cc6c5)n4)c3c2c1 ; [2H]c1cc2c(oc3c([2H])cc([2H])c(-c4ccc(-c5nc(-c6ccccc6)nc(-c6ccc7sc8ccccc8c7c6)n5)c5ccccc45)c32)c([2H])c1[2H] ; [2H]c1cc([2H])c2oc3ccc([2H])c(-c4nc(-c5ccc([Si](c6ccccc6)(c6ccccc6)c6ccccc6)cc5)nc(-c5ccc6ccccc6c5)n4)c3c2c1]

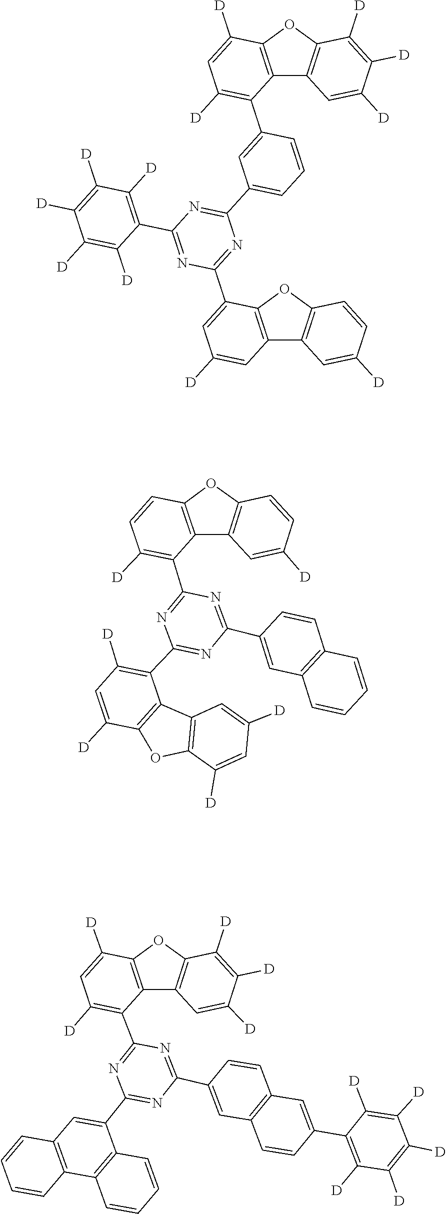 [SMILES: [2H]c1ccc2oc3c(-c4nc(-c5cccc(-c6c([2H])cc([2H])c7oc8c([2H])c([2H])c([2H])cc8c67)c5)nc(-c5c([2H])c([2H])c([2H])c([2H])c5[2H])n4)cc([2H])cc3c2c1 ; [2H]c1ccc2oc3ccc([2H])c(-c4nc(-c5ccc6ccccc6c5)nc(-c5c([2H])cc([2H])c6oc7c([2H])cc([2H])cc7c56)n4)c3c2c1 ; [2H]c1cc2c(oc3c([2H])cc([2H])c(-c4nc(-c5ccc6cc(-c7c([2H])c([2H])c([2H])c([2H])c7[2H])ccc6c5)nc(-c5cc6ccccc6c6ccccc56)n4)c32)c([2H])c1[2H]]

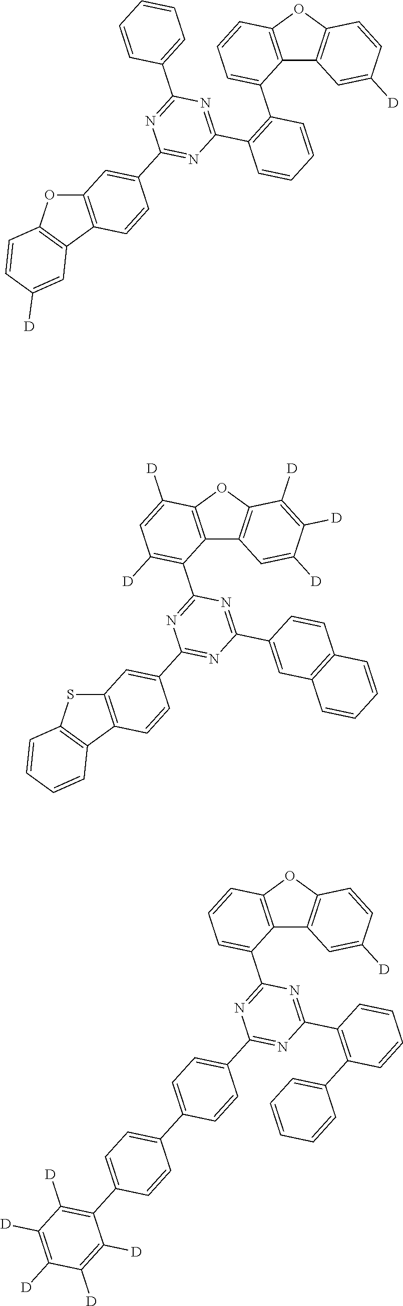 [SMILES: [2H]c1ccc2oc3cc(-c4nc(-c5ccccc5)nc(-c5ccccc5-c5cccc6oc7ccc([2H])cc7c56)n4)ccc3c2c1 ; [2H]c1cc2c(oc3c([2H])cc([2H])c(-c4nc(-c5ccc6ccccc6c5)nc(-c5ccc6c(c5)sc5ccccc56)n4)c32)c([2H])c1[2H] ; [2H]c1ccc2oc3cccc(-c4nc(-c5ccc(-c6ccc(-c7c([2H])c([2H])c([2H])c([2H])c7[2H])cc6)cc5)nc(-c5ccccc5-c5ccccc5)n4)c3c2c1]

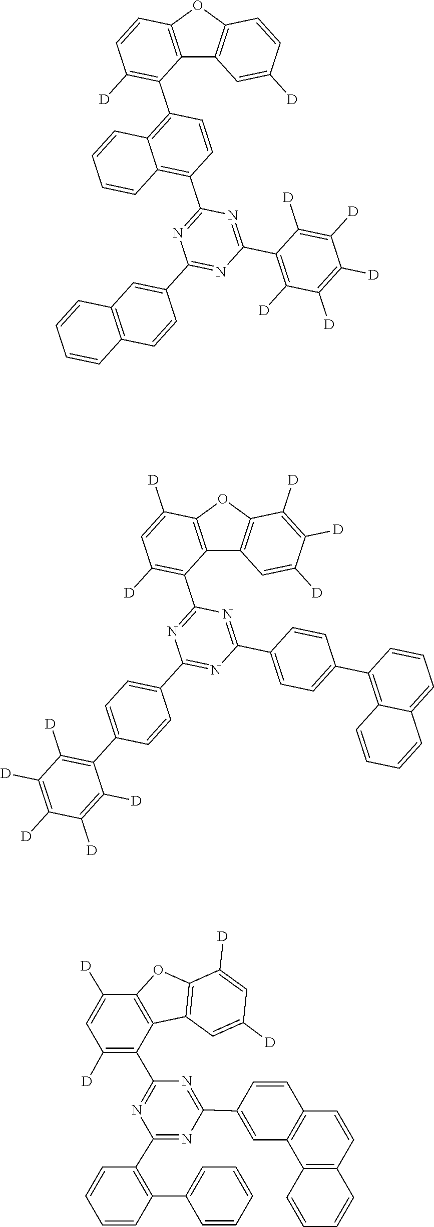 [SMILES: [2H]c1ccc2oc3ccc([2H])c(-c4ccc(-c5nc(-c6ccc7ccccc7c6)nc(-c6c([2H])c([2H])c([2H])c([2H])c6[2H])n5)c5ccccc45)c3c2c1 ; [2H]c1cc2c(oc3c([2H])cc([2H])c(-c4nc(-c5ccc(-c6c([2H])c([2H])c([2H])c([2H])c6[2H])cc5)nc(-c5ccc(-c6cccc7ccccc67)cc5)n4)c32)c([2H])c1[2H] ; [2H]c1cc([2H])c2oc3c([2H])cc([2H])c(-c4nc(-c5ccc6ccc7ccccc7c6c5)nc(-c5ccccc5-c5ccccc5)n4)c3c2c1]

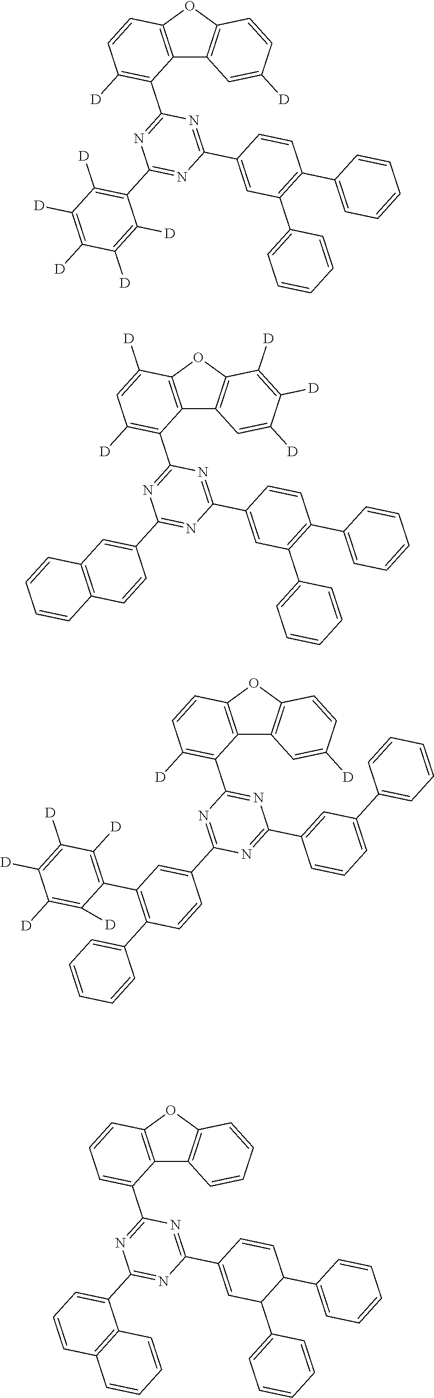 [SMILES: [2H]c1ccc2oc3ccc([2H])c(-c4nc(-c5ccc(-c6ccccc6)c(-c6ccccc6)c5)nc(-c5c([2H])c([2H])c([2H])c([2H])c5[2H])n4)c3c2c1 ; [2H]c1cc2c(oc3c([2H])cc([2H])c(-c4nc(-c5ccc(-c6ccccc6)c(-c6ccccc6)c5)nc(-c5ccc6ccccc6c5)n4)c32)c([2H])c1[2H] ; [2H]c1ccc2oc3ccc([2H])c(-c4nc(-c5cccc(-c6ccccc6)c5)nc(-c5ccc(-c6ccccc6)c(-c6c([2H])c([2H])c([2H])c([2H])c6[2H])c5)n4)c3c2c1 ; C1=CC(c2ccccc2)C(c2ccccc2)C=C1c1nc(-c2cccc3ccccc23)nc(-c2cccc3oc4ccccc4c23)n1]

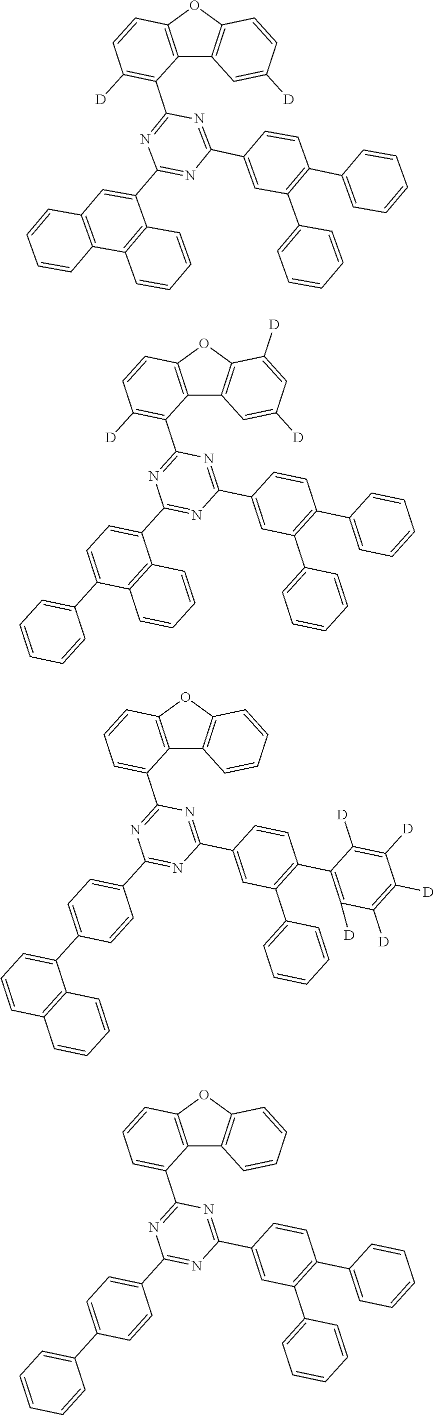

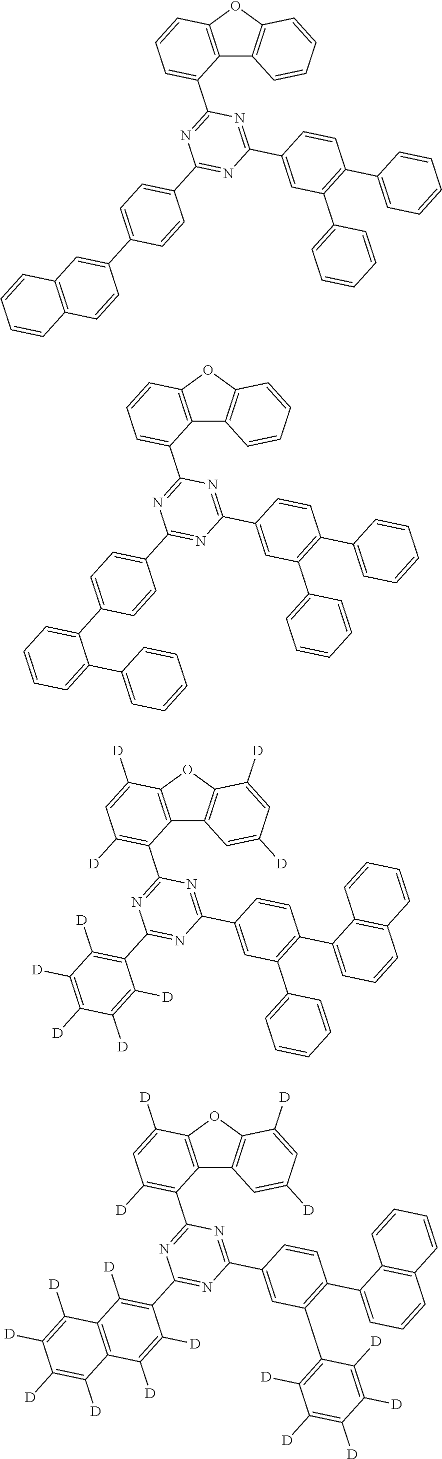

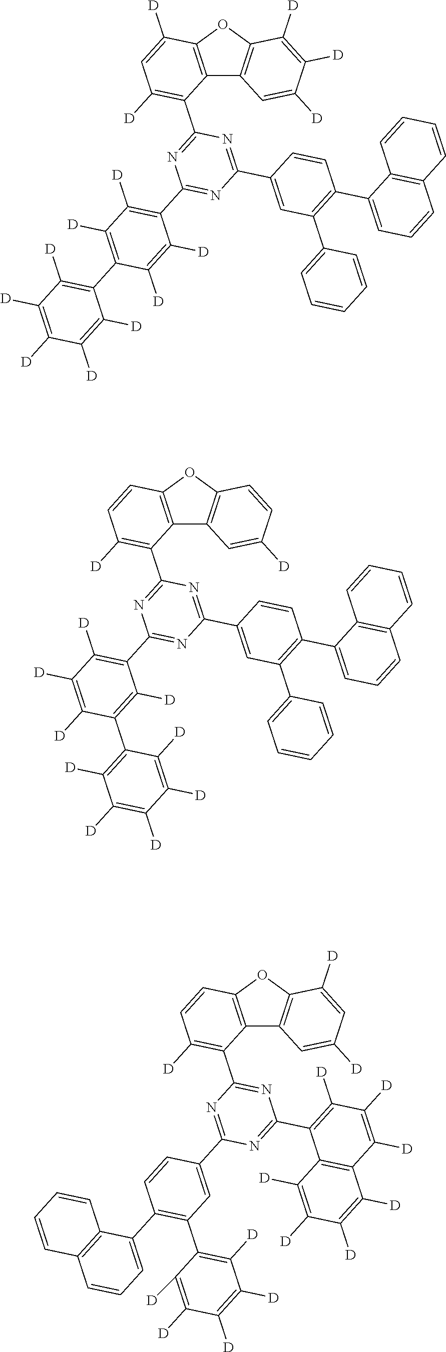 [SMILES: [2H]c1cc2c(oc3c([2H])cc([2H])c(-c4nc(-c5ccc(-c6cccc7ccccc67)c(-c6ccccc6)c5)nc(-c5c([2H])c([2H])c(-c6c([2H])c([2H])c([2H])c([2H])c6[2H])c([2H])c5[2H])n4)c32)c([2H])c1[2H] ; [2H]c1ccc2oc3ccc([2H])c(-c4nc(-c5ccc(-c6cccc7ccccc67)c(-c6ccccc6)c5)nc(-c5c([2H])c([2H])c([2H])c(-c6c([2H])c([2H])c([2H])c([2H])c6[2H])c5[2H])n4)c3c2c1 ; [2H]c1cc([2H])c2oc3ccc([2H])c(-c4nc(-c5ccc(-c6cccc7ccccc67)c(-c6c([2H])c([2H])c([2H])c([2H])c6[2H])c5)nc(-c5c([2H])c([2H])c([2H])c6c([2H])c([2H])c([2H])c([2H])c56)n4)c3c2c1]

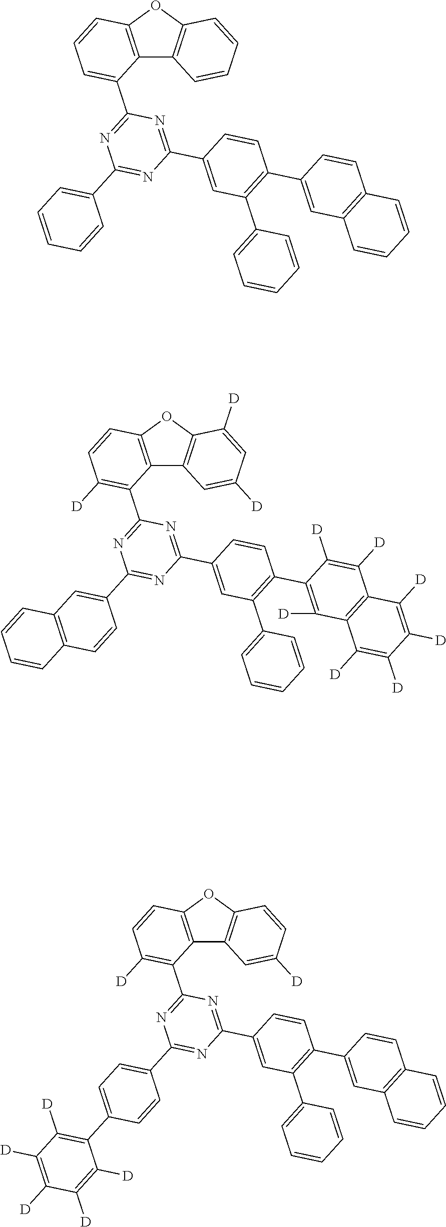 [SMILES: c1ccc(-c2nc(-c3ccc(-c4ccc5ccccc5c4)c(-c4ccccc4)c3)nc(-c3cccc4oc5ccccc5c34)n2)cc1 ; [2H]c1cc([2H])c2oc3ccc([2H])c(-c4nc(-c5ccc(-c6c([2H])c([2H])c7c([2H])c([2H])c([2H])c([2H])c7c6[2H])c(-c6ccccc6)c5)nc(-c5ccc6ccccc6c5)n4)c3c2c1 ; [2H]c1ccc2oc3ccc([2H])c(-c4nc(-c5ccc(-c6c([2H])c([2H])c([2H])c([2H])c6[2H])cc5)nc(-c5ccc(-c6ccc7ccccc7c6)c(-c6ccccc6)c5)n4)c3c2c1]

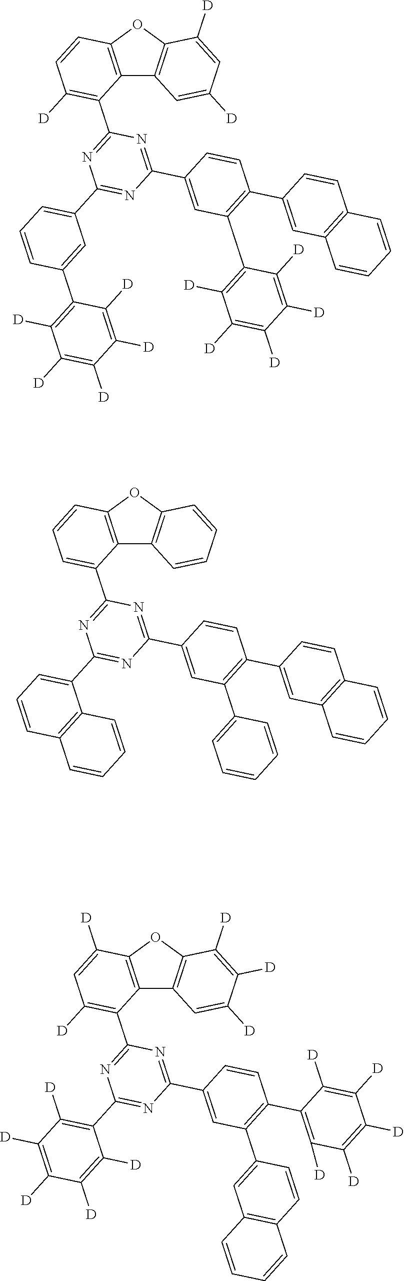

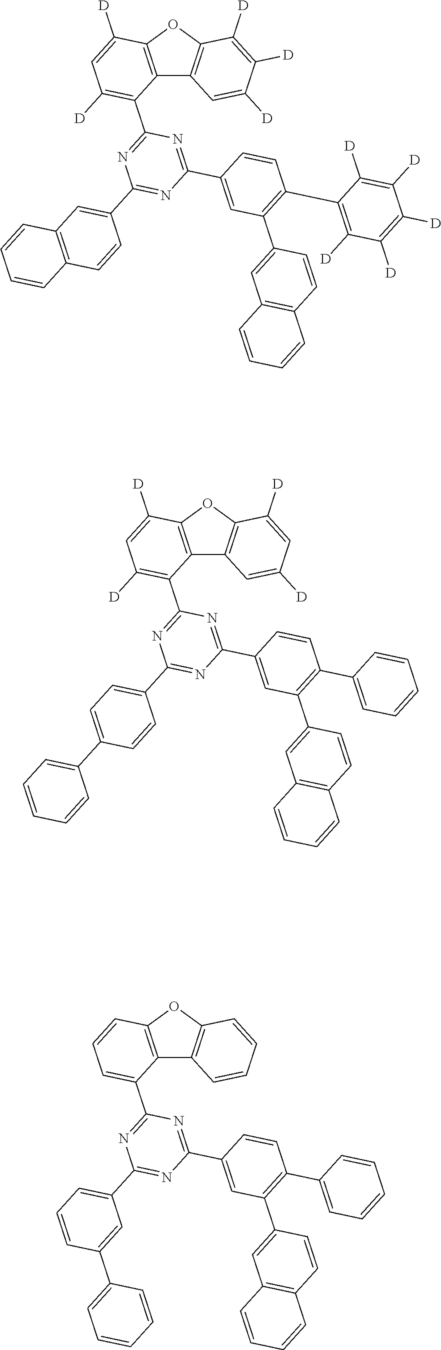

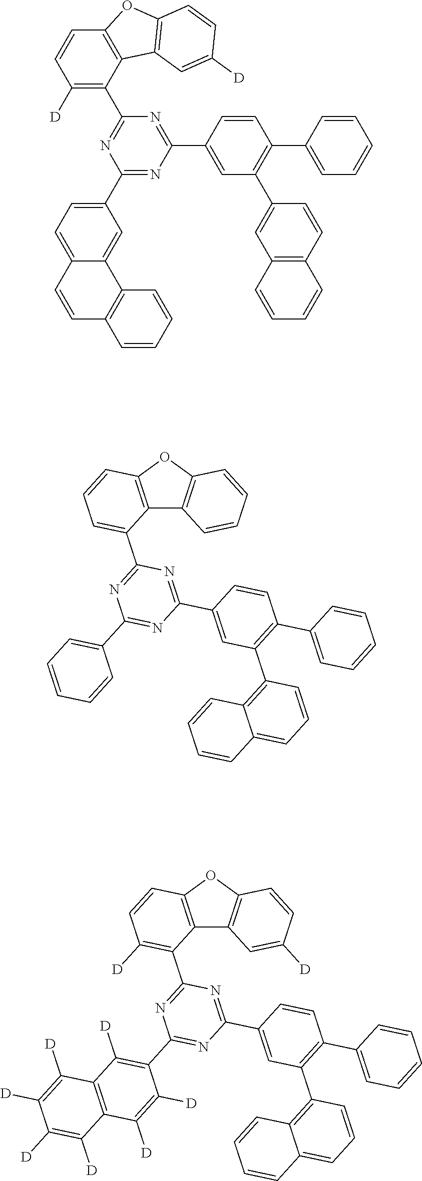 [SMILES: [2H]c1ccc2oc3ccc([2H])c(-c4nc(-c5ccc(-c6ccccc6)c(-c6ccc7ccccc7c6)c5)nc(-c5ccc6ccc7ccccc7c6c5)n4)c3c2c1 ; c1ccc(-c2nc(-c3ccc(-c4ccccc4)c(-c4cccc5ccccc45)c3)nc(-c3cccc4oc5ccccc5c34)n2)cc1 ; [2H]c1ccc2oc3ccc([2H])c(-c4nc(-c5ccc(-c6ccccc6)c(-c6cccc7ccccc67)c5)nc(-c5c([2H])c([2H])c6c([2H])c([2H])c([2H])c([2H])c6c5[2H])n4)c3c2c1]

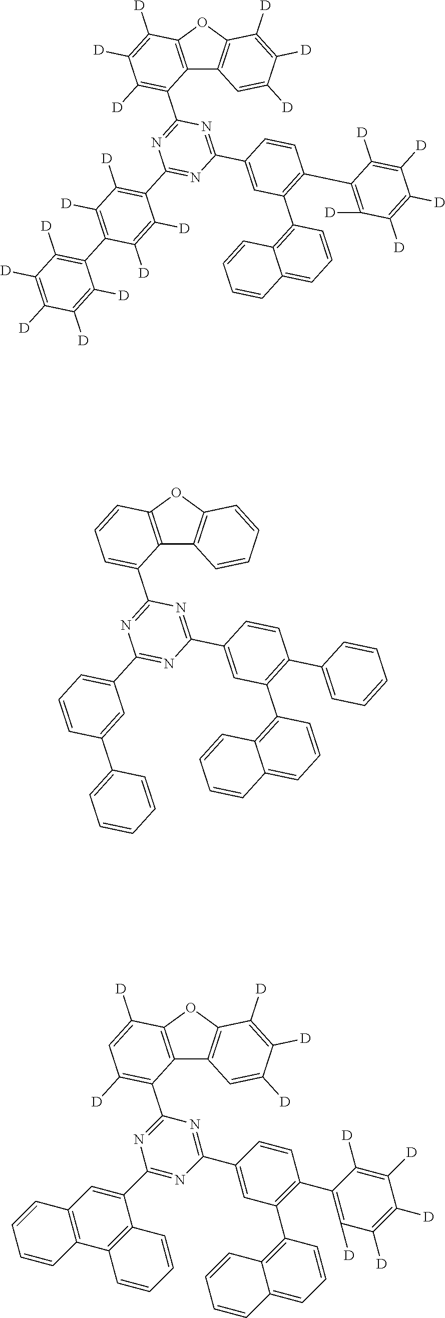 [SMILES: [2H]c1cc2c(oc3c([2H])c([2H])c([2H])c(-c4nc(-c5ccc(-c6c([2H])c([2H])c([2H])c([2H])c6[2H])c(-c6cccc7ccccc67)c5)nc(-c5c([2H])c([2H])c(-c6c([2H])c([2H])c([2H])c([2H])c6[2H])c([2H])c5[2H])n4)c32)c([2H])c1[2H] ; c1ccc(-c2cccc(-c3nc(-c4ccc(-c5ccccc5)c(-c5cccc6ccccc56)c4)nc(-c4cccc5oc6ccccc6c45)n3)c2)cc1 ; [2H]c1cc2c(oc3c([2H])cc([2H])c(-c4nc(-c5ccc(-c6c([2H])c([2H])c([2H])c([2H])c6[2H])c(-c6cccc7ccccc67)c5)nc(-c5cc6ccccc6c6ccccc56)n4)c32)c([2H])c1[2H]]

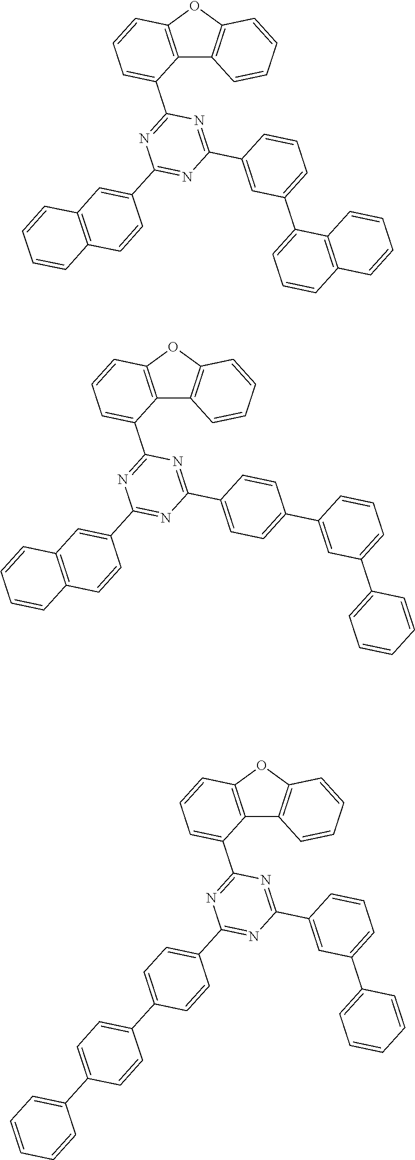 [SMILES: c1cc(-c2nc(-c3ccc4ccccc4c3)nc(-c3cccc4oc5ccccc5c34)n2)cc(-c2cccc3ccccc23)c1 ; c1ccc(-c2cccc(-c3ccc(-c4nc(-c5ccc6ccccc6c5)nc(-c5cccc6oc7ccccc7c56)n4)cc3)c2)cc1 ; c1ccc(-c2ccc(-c3ccc(-c4nc(-c5cccc(-c6ccccc6)c5)nc(-c5cccc6oc7ccccc7c56)n4)cc3)cc2)cc1]

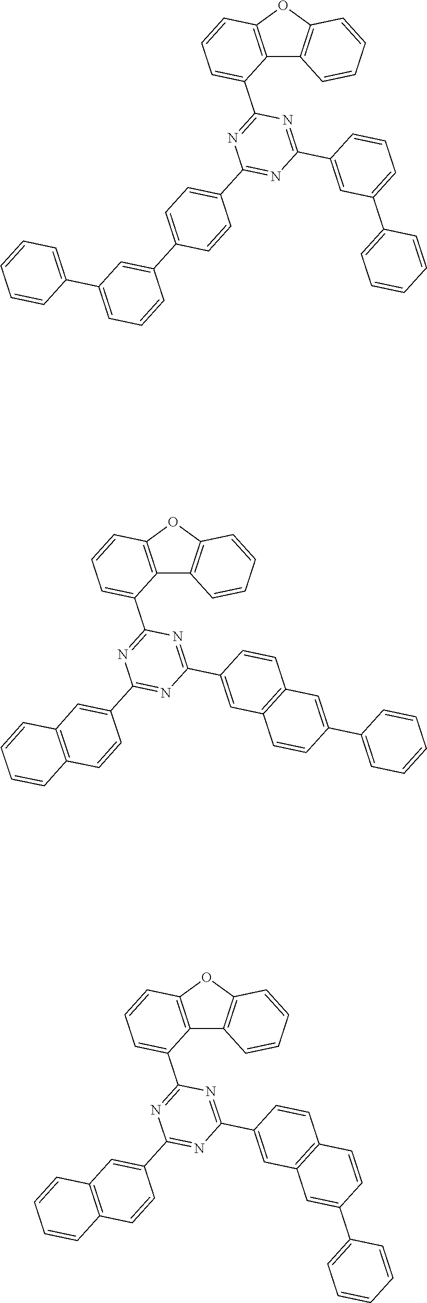 [SMILES: c1ccc(-c2cccc(-c3ccc(-c4nc(-c5cccc(-c6ccccc6)c5)nc(-c5cccc6oc7ccccc7c56)n4)cc3)c2)cc1 ; c1ccc(-c2ccc3cc(-c4nc(-c5ccc6ccccc6c5)nc(-c5cccc6oc7ccccc7c56)n4)ccc3c2)cc1 ; c1ccc(-c2ccc3ccc(-c4nc(-c5ccc6ccccc6c5)nc(-c5cccc6oc7ccccc7c56)n4)cc3c2)cc1]

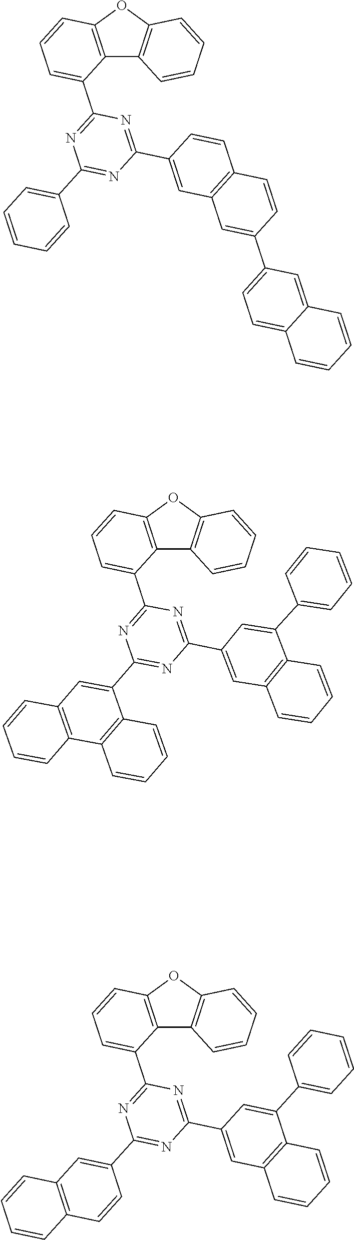 [SMILES: c1ccc(-c2nc(-c3ccc4ccc(-c5ccc6ccccc6c5)cc4c3)nc(-c3cccc4oc5ccccc5c34)n2)cc1 ; c1ccc(-c2cc(-c3nc(-c4cc5ccccc5c5ccccc45)nc(-c4cccc5oc6ccccc6c45)n3)cc3ccccc23)cc1 ; c1ccc(-c2cc(-c3nc(-c4ccc5ccccc5c4)nc(-c4cccc5oc6ccccc6c45)n3)cc3ccccc23)cc1]

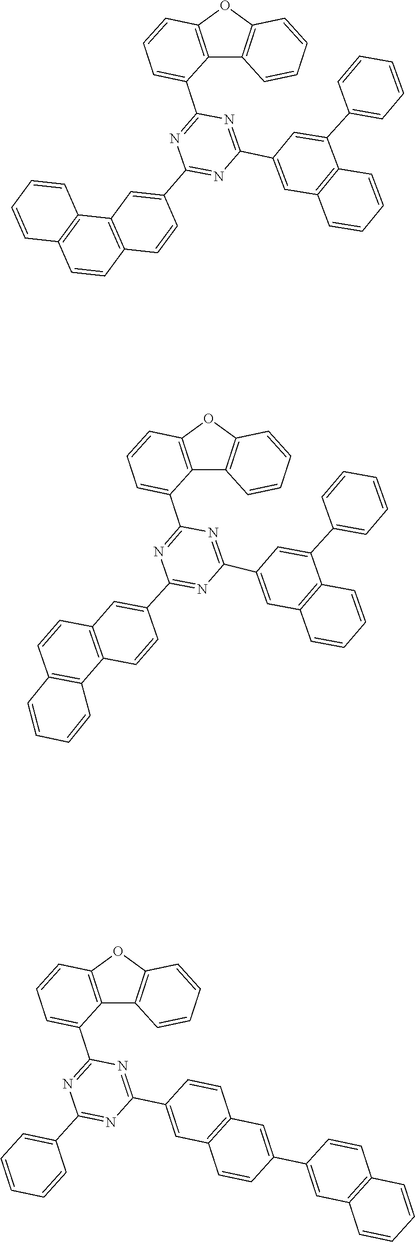 [SMILES: c1ccc(-c2cc(-c3nc(-c4ccc5ccc6ccccc6c5c4)nc(-c4cccc5oc6ccccc6c45)n3)cc3ccccc23)cc1 ; c1ccc(-c2cc(-c3nc(-c4ccc5c(ccc6ccccc65)c4)nc(-c4cccc5oc6ccccc6c45)n3)cc3ccccc23)cc1 ; c1ccc(-c2nc(-c3ccc4cc(-c5ccc6ccccc6c5)ccc4c3)nc(-c3cccc4oc5ccccc5c34)n2)cc1]

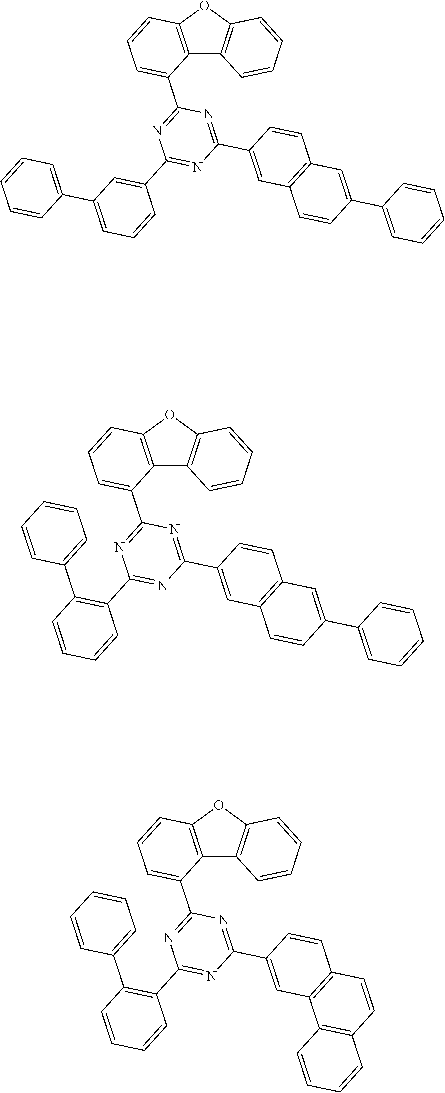 [SMILES: c1ccc(-c2cccc(-c3nc(-c4ccc5cc(-c6ccccc6)ccc5c4)nc(-c4cccc5oc6ccccc6c45)n3)c2)cc1 ; c1ccc(-c2ccc3cc(-c4nc(-c5ccccc5-c5ccccc5)nc(-c5cccc6oc7ccccc7c56)n4)ccc3c2)cc1 ; c1ccc(-c2ccccc2-c2nc(-c3ccc4ccc5ccccc5c4c3)nc(-c3cccc4oc5ccccc5c34)n2)cc1]

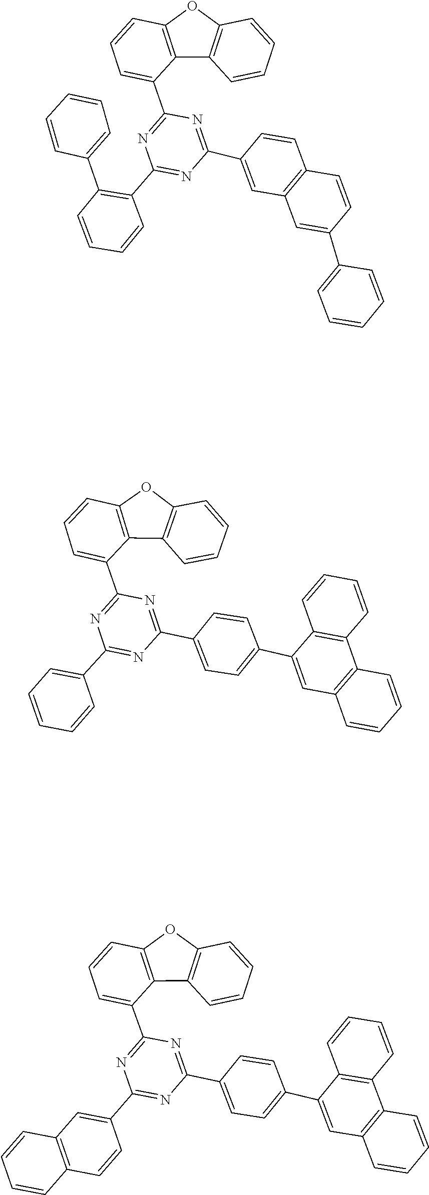 [SMILES: c1ccc(-c2ccc3ccc(-c4nc(-c5ccccc5-c5ccccc5)nc(-c5cccc6oc7ccccc7c56)n4)cc3c2)cc1 ; c1ccc(-c2nc(-c3ccc(-c4cc5ccccc5c5ccccc45)cc3)nc(-c3cccc4oc5ccccc5c34)n2)cc1 ; c1ccc2cc(-c3nc(-c4ccc(-c5cc6ccccc6c6ccccc56)cc4)nc(-c4cccc5oc6ccccc6c45)n3)ccc2c1]

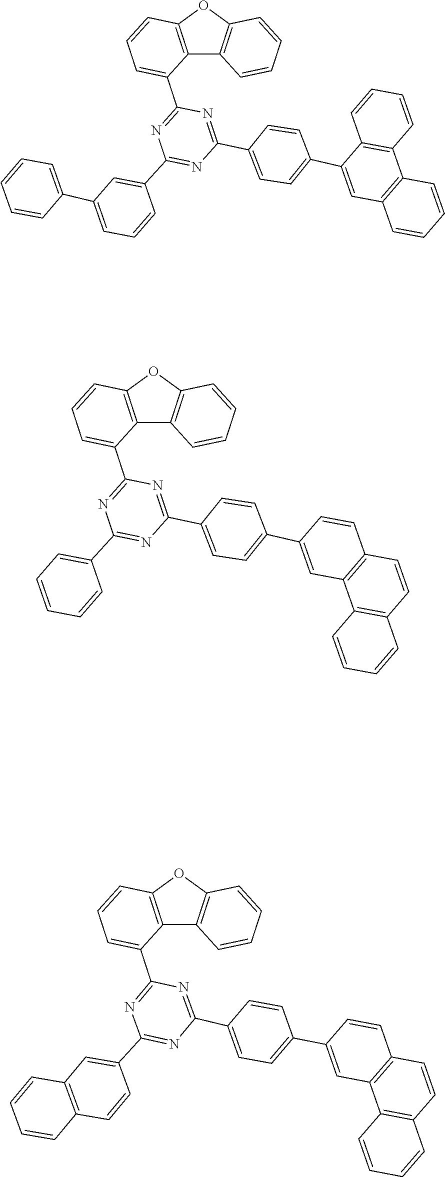 [SMILES: c1ccc(-c2cccc(-c3nc(-c4ccc(-c5cc6ccccc6c6ccccc56)cc4)nc(-c4cccc5oc6ccccc6c45)n3)c2)cc1 ; c1ccc(-c2nc(-c3ccc(-c4ccc5ccc6ccccc6c5c4)cc3)nc(-c3cccc4oc5ccccc5c34)n2)cc1 ; c1ccc2cc(-c3nc(-c4ccc(-c5ccc6ccc7ccccc7c6c5)cc4)nc(-c4cccc5oc6ccccc6c45)n3)ccc2c1]

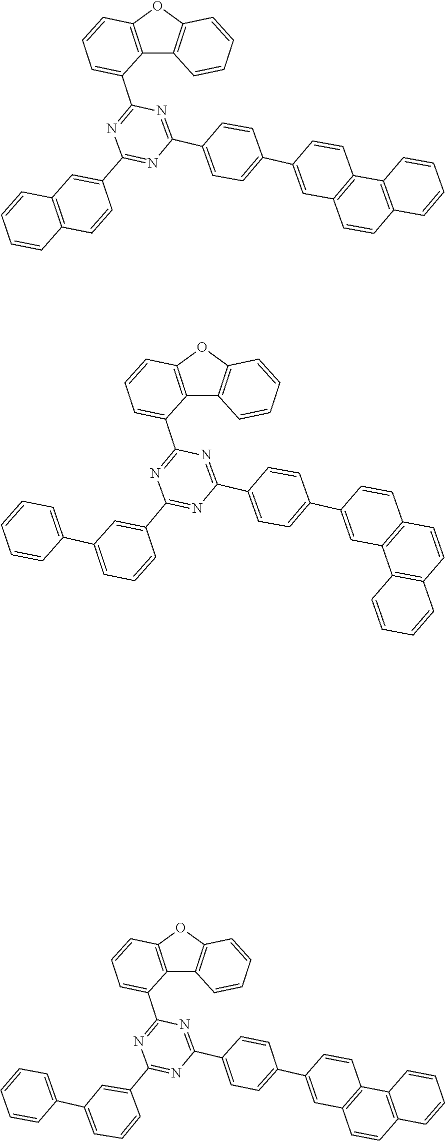 [SMILES: c1ccc2cc(-c3nc(-c4ccc(-c5ccc6c(ccc7ccccc76)c5)cc4)nc(-c4cccc5oc6ccccc6c45)n3)ccc2c1 ; c1ccc(-c2cccc(-c3nc(-c4ccc(-c5ccc6ccc7ccccc7c6c5)cc4)nc(-c4cccc5oc6ccccc6c45)n3)c2)cc1 ; c1ccc(-c2cccc(-c3nc(-c4ccc(-c5ccc6c(ccc7ccccc76)c5)cc4)nc(-c4cccc5oc6ccccc6c45)n3)c2)cc1]

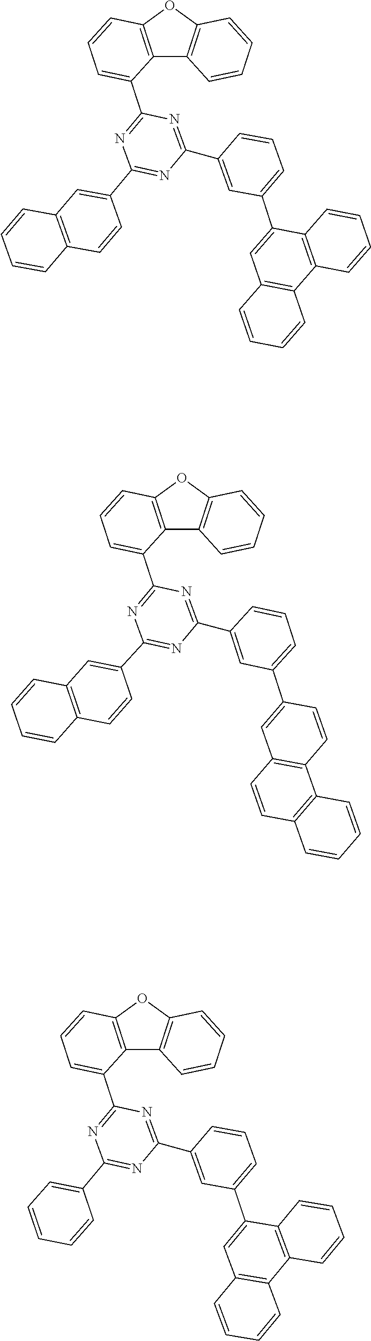 [SMILES: c1cc(-c2nc(-c3ccc4ccccc4c3)nc(-c3cccc4oc5ccccc5c34)n2)cc(-c2cc3ccccc3c3ccccc23)c1 ; c1cc(-c2ccc3c(ccc4ccccc43)c2)cc(-c2nc(-c3ccc4ccccc4c3)nc(-c3cccc4oc5ccccc5c34)n2)c1 ; c1ccc(-c2nc(-c3cccc(-c4cc5ccccc5c5ccccc45)c3)nc(-c3cccc4oc5ccccc5c34)n2)cc1]

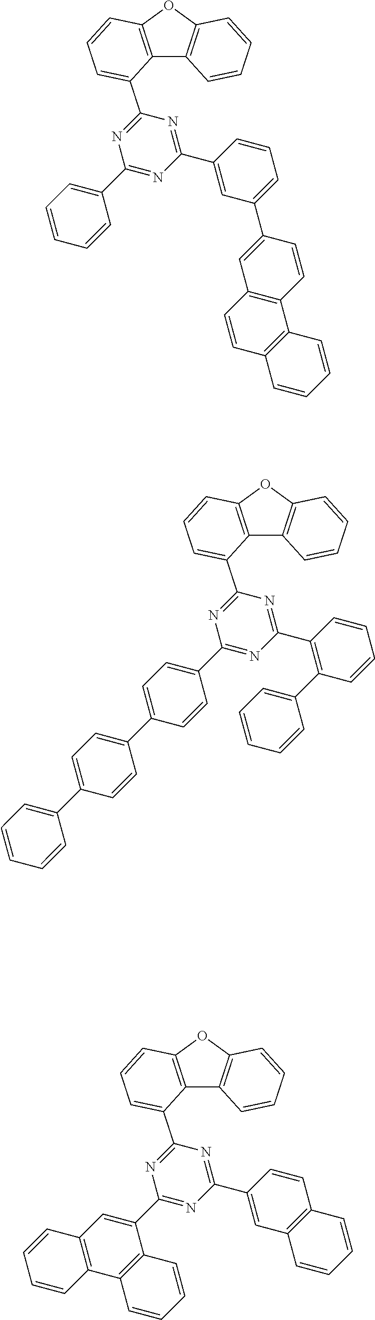 [SMILES: c1ccc(-c2nc(-c3cccc(-c4ccc5c(ccc6ccccc65)c4)c3)nc(-c3cccc4oc5ccccc5c34)n2)cc1 ; c1ccc(-c2ccc(-c3ccc(-c4nc(-c5ccccc5-c5ccccc5)nc(-c5cccc6oc7ccccc7c56)n4)cc3)cc2)cc1 ; c1ccc2cc(-c3nc(-c4cc5ccccc5c5ccccc45)nc(-c4cccc5oc6ccccc6c45)n3)ccc2c1]

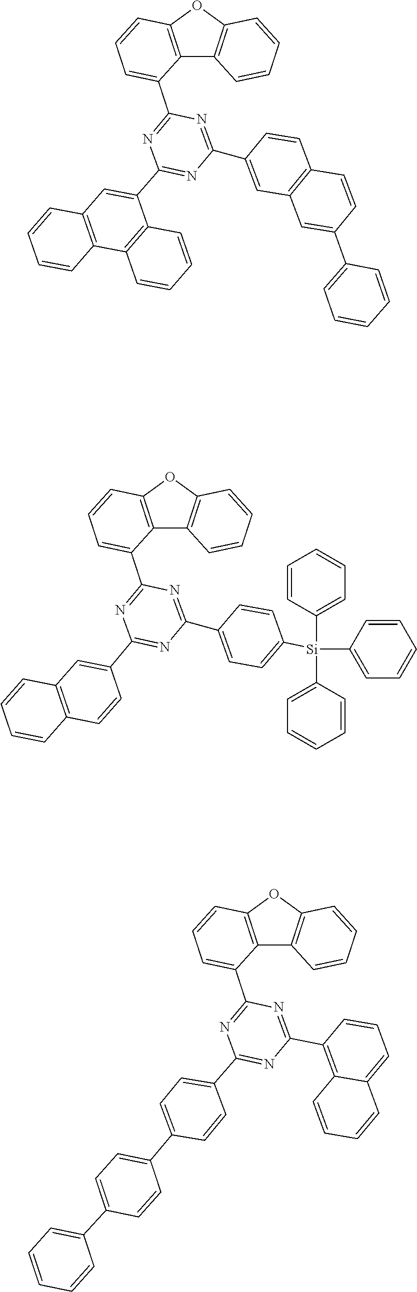 [SMILES: c1ccc(-c2ccc3ccc(-c4nc(-c5cc6ccccc6c6ccccc56)nc(-c5cccc6oc7ccccc7c56)n4)cc3c2)cc1 ; c1ccc([Si](c2ccccc2)(c2ccccc2)c2ccc(-c3nc(-c4ccc5ccccc5c4)nc(-c4cccc5oc6ccccc6c45)n3)cc2)cc1 ; c1ccc(-c2ccc(-c3ccc(-c4nc(-c5cccc6ccccc56)nc(-c5cccc6oc7ccccc7c56)n4)cc3)cc2)cc1]

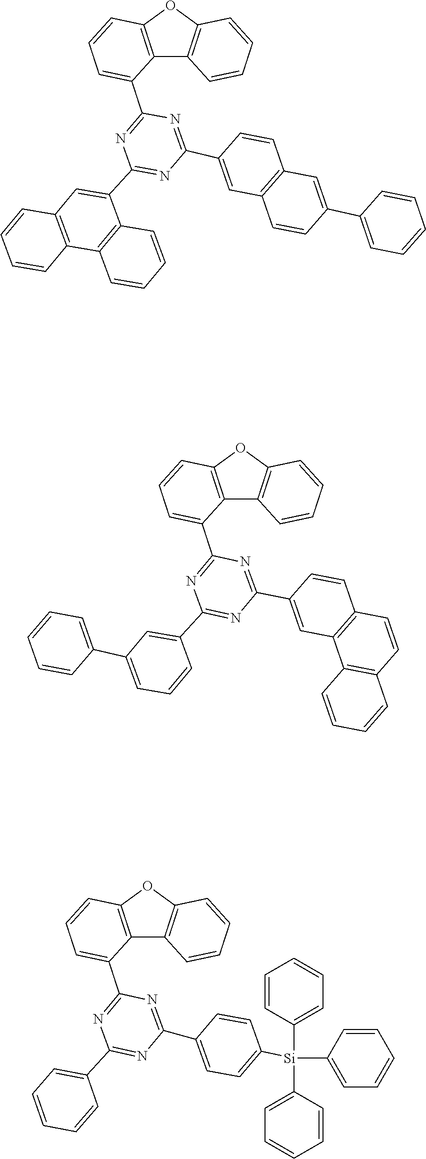 [SMILES: c1ccc(-c2ccc3cc(-c4nc(-c5cc6ccccc6c6ccccc56)nc(-c5cccc6oc7ccccc7c56)n4)ccc3c2)cc1 ; c1ccc(-c2cccc(-c3nc(-c4ccc5ccc6ccccc6c5c4)nc(-c4cccc5oc6ccccc6c45)n3)c2)cc1 ; c1ccc(-c2nc(-c3ccc([Si](c4ccccc4)(c4ccccc4)c4ccccc4)cc3)nc(-c3cccc4oc5ccccc5c34)n2)cc1]

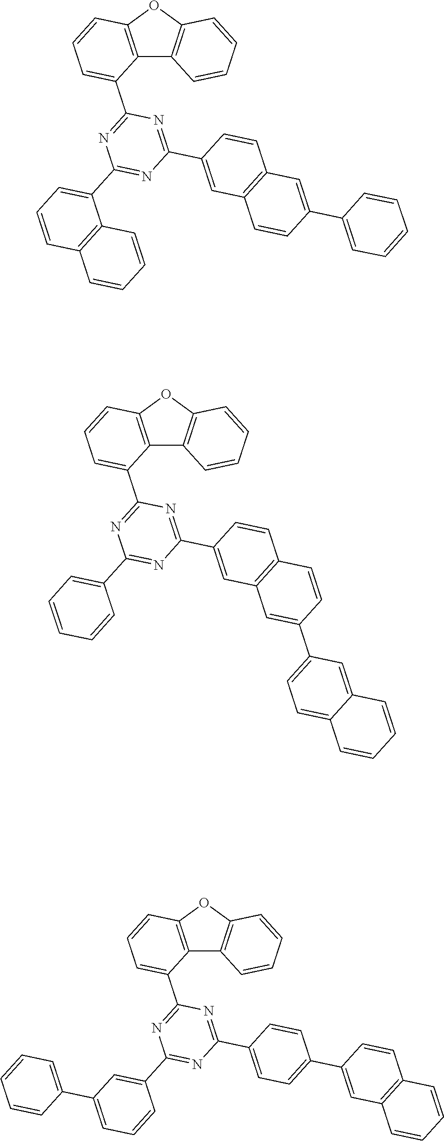 [SMILES: c1ccc(-c2ccc3cc(-c4nc(-c5cccc6ccccc56)nc(-c5cccc6oc7ccccc7c56)n4)ccc3c2)cc1 ; c1ccc(-c2nc(-c3ccc4ccc(-c5ccc6ccccc6c5)cc4c3)nc(-c3cccc4oc5ccccc5c34)n2)cc1 ; c1ccc(-c2cccc(-c3nc(-c4ccc(-c5ccc6ccccc6c5)cc4)nc(-c4cccc5oc6ccccc6c45)n3)c2)cc1]

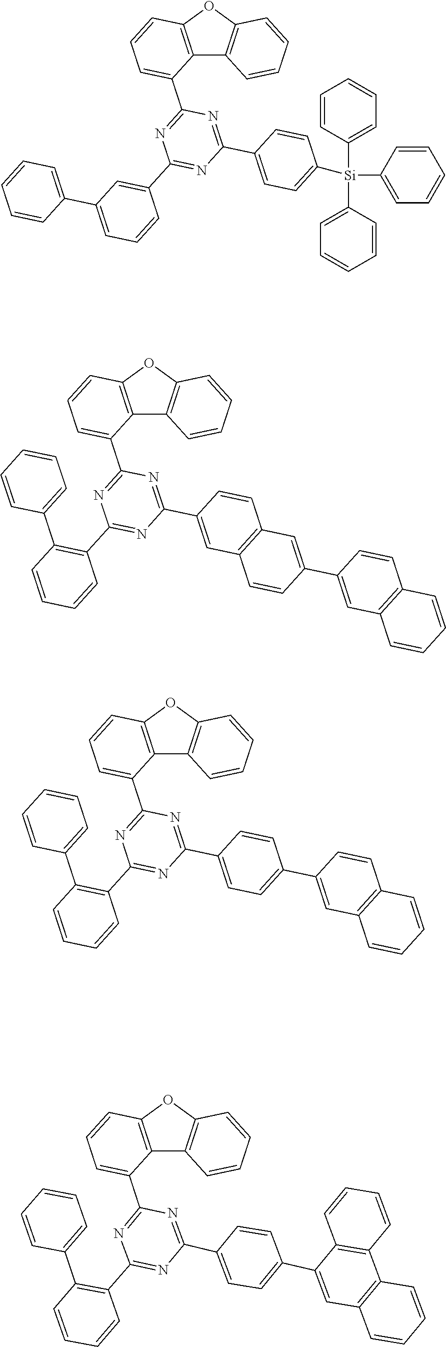 [SMILES: c1ccc(-c2cccc(-c3nc(-c4ccc([Si](c5ccccc5)(c5ccccc5)c5ccccc5)cc4)nc(-c4cccc5oc6ccccc6c45)n3)c2)cc1 ; c1ccc(-c2ccccc2-c2nc(-c3ccc4cc(-c5ccc6ccccc6c5)ccc4c3)nc(-c3cccc4oc5ccccc5c34)n2)cc1 ; c1ccc(-c2ccccc2-c2nc(-c3ccc(-c4ccc5ccccc5c4)cc3)nc(-c3cccc4oc5ccccc5c34)n2)cc1 ; c1ccc(-c2ccccc2-c2nc(-c3ccc(-c4cc5ccccc5c5ccccc45)cc3)nc(-c3cccc4oc5ccccc5c34)n2)cc1]

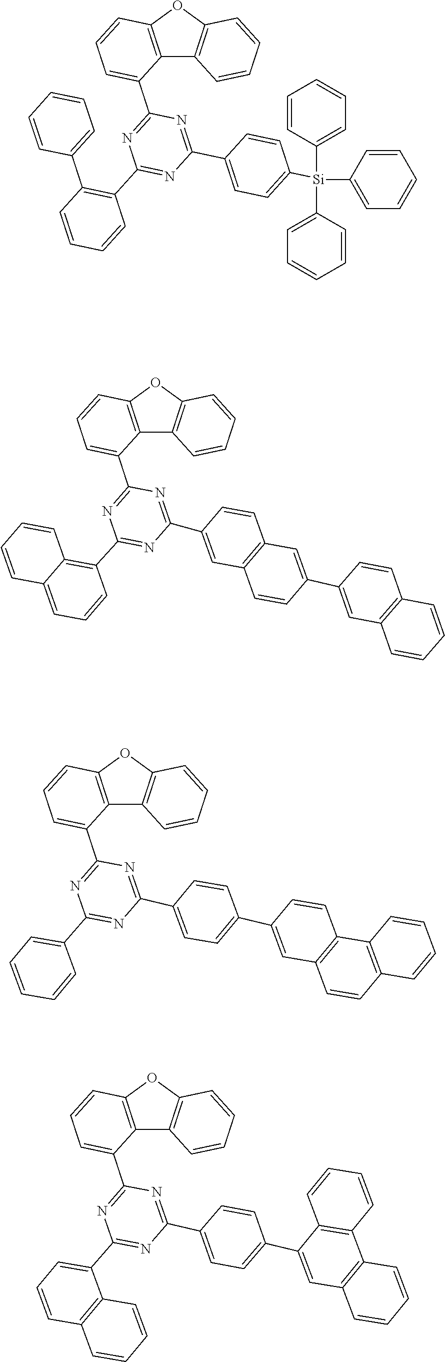 [SMILES: c1ccc(-c2ccccc2-c2nc(-c3ccc([Si](c4ccccc4)(c4ccccc4)c4ccccc4)cc3)nc(-c3cccc4oc5ccccc5c34)n2)cc1 ; c1ccc2cc(-c3ccc4cc(-c5nc(-c6cccc7ccccc67)nc(-c6cccc7oc8ccccc8c67)n5)ccc4c3)ccc2c1 ; c1ccc(-c2nc(-c3ccc(-c4ccc5c(ccc6ccccc65)c4)cc3)nc(-c3cccc4oc5ccccc5c34)n2)cc1 ; c1ccc2c(-c3nc(-c4ccc(-c5cc6ccccc6c6ccccc56)cc4)nc(-c4cccc5oc6ccccc6c45)n3)cccc2c1]

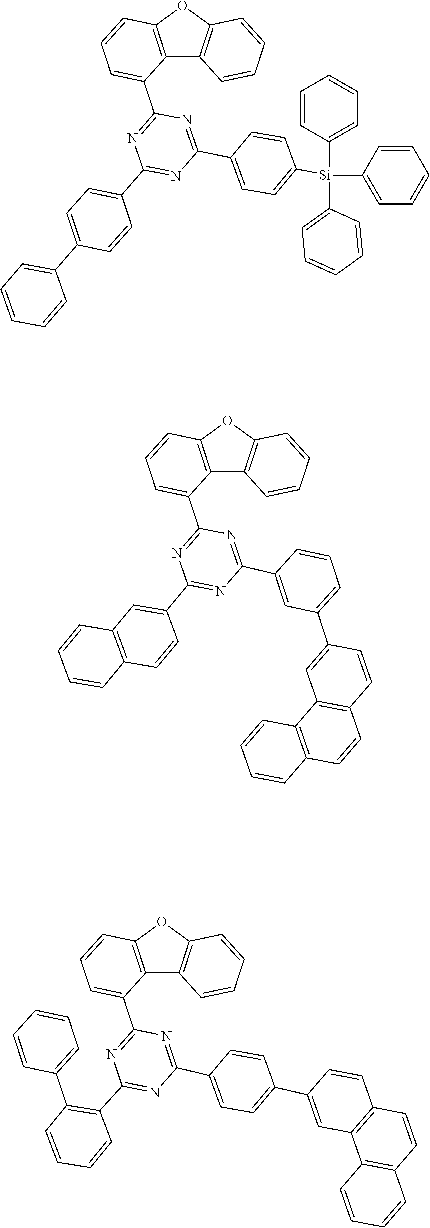 [SMILES: c1ccc(-c2ccc(-c3nc(-c4ccc([Si](c5ccccc5)(c5ccccc5)c5ccccc5)cc4)nc(-c4cccc5oc6ccccc6c45)n3)cc2)cc1 ; c1cc(-c2ccc3ccc4ccccc4c3c2)cc(-c2nc(-c3ccc4ccccc4c3)nc(-c3cccc4oc5ccccc5c34)n2)c1 ; c1ccc(-c2ccccc2-c2nc(-c3ccc(-c4ccc5ccc6ccccc6c5c4)cc3)nc(-c3cccc4oc5ccccc5c34)n2)cc1]

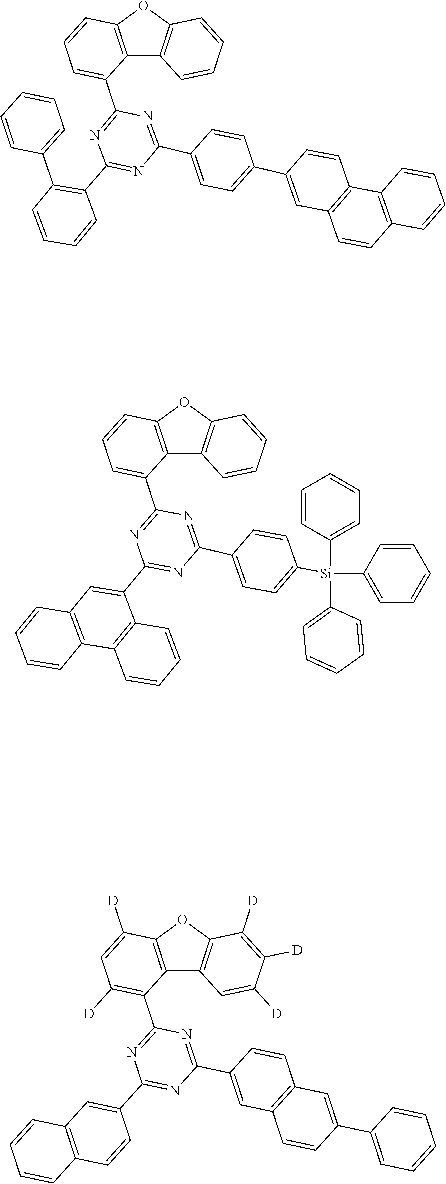 [SMILES: c1ccc(-c2ccccc2-c2nc(-c3ccc(-c4ccc5c(ccc6ccccc65)c4)cc3)nc(-c3cccc4oc5ccccc5c34)n2)cc1 ; c1ccc([Si](c2ccccc2)(c2ccccc2)c2ccc(-c3nc(-c4cc5ccccc5c5ccccc45)nc(-c4cccc5oc6ccccc6c45)n3)cc2)cc1 ; [2H]c1cc2c(oc3c([2H])cc([2H])c(-c4nc(-c5ccc6ccccc6c5)nc(-c5ccc6cc(-c7ccccc7)ccc6c5)n4)c32)c([2H])c1[2H]]

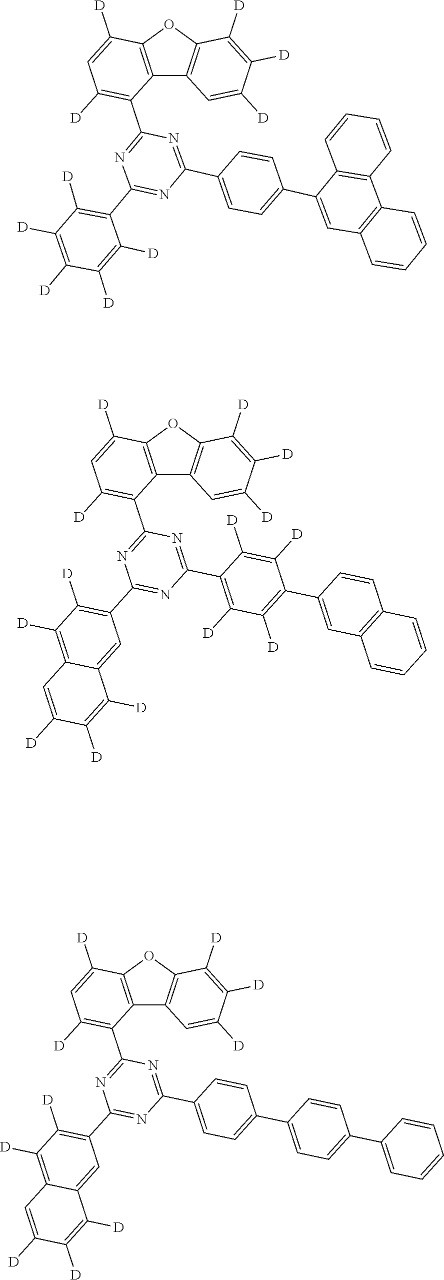 [SMILES: [2H]c1cc2c(oc3c([2H])cc([2H])c(-c4nc(-c5ccc(-c6cc7ccccc7c7ccccc67)cc5)nc(-c5c([2H])c([2H])c([2H])c([2H])c5[2H])n4)c32)c([2H])c1[2H] ; [2H]c1cc2c([2H])c([2H])c(-c3nc(-c4c([2H])c([2H])c(-c5ccc6ccccc6c5)c([2H])c4[2H])nc(-c4c([2H])cc([2H])c5oc6c([2H])c([2H])c([2H])cc6c45)n3)cc2c([2H])c1[2H] ; [2H]c1cc2c([2H])c([2H])c(-c3nc(-c4ccc(-c5ccc(-c6ccccc6)cc5)cc4)nc(-c4c([2H])cc([2H])c5oc6c([2H])c([2H])c([2H])cc6c45)n3)cc2c([2H])c1[2H]]

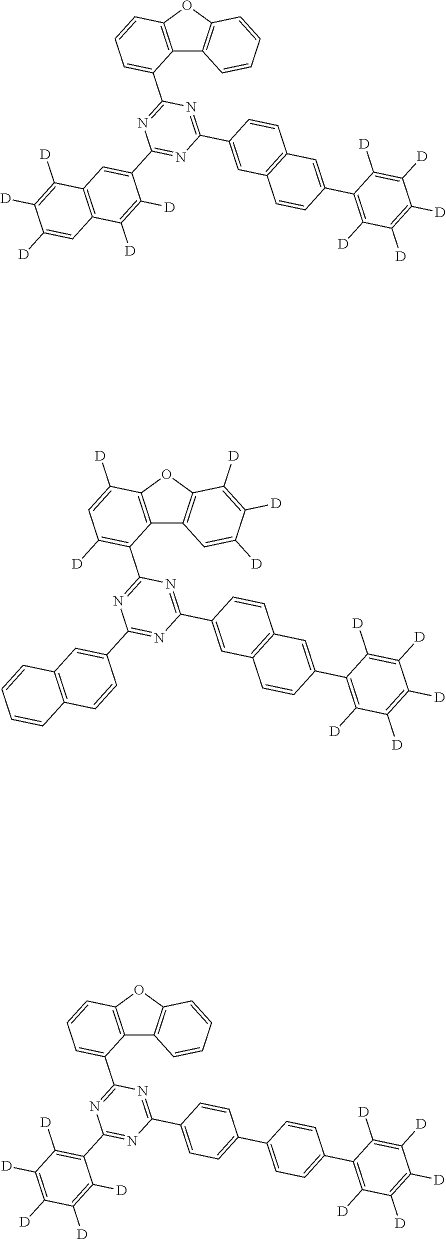 [SMILES: [2H]c1cc2c([2H])c([2H])c(-c3nc(-c4ccc5cc(-c6c([2H])c([2H])c([2H])c([2H])c6[2H])ccc5c4)nc(-c4cccc5oc6ccccc6c45)n3)cc2c([2H])c1[2H] ; [2H]c1cc2c(oc3c([2H])cc([2H])c(-c4nc(-c5ccc6ccccc6c5)nc(-c5ccc6cc(-c7c([2H])c([2H])c([2H])c([2H])c7[2H])ccc6c5)n4)c32)c([2H])c1[2H] ; [2H]c1c([2H])c([2H])c(-c2ccc(-c3ccc(-c4nc(-c5c([2H])c([2H])c([2H])c([2H])c5[2H])nc(-c5cccc6oc7ccccc7c56)n4)cc3)cc2)c([2H])c1[2H]]

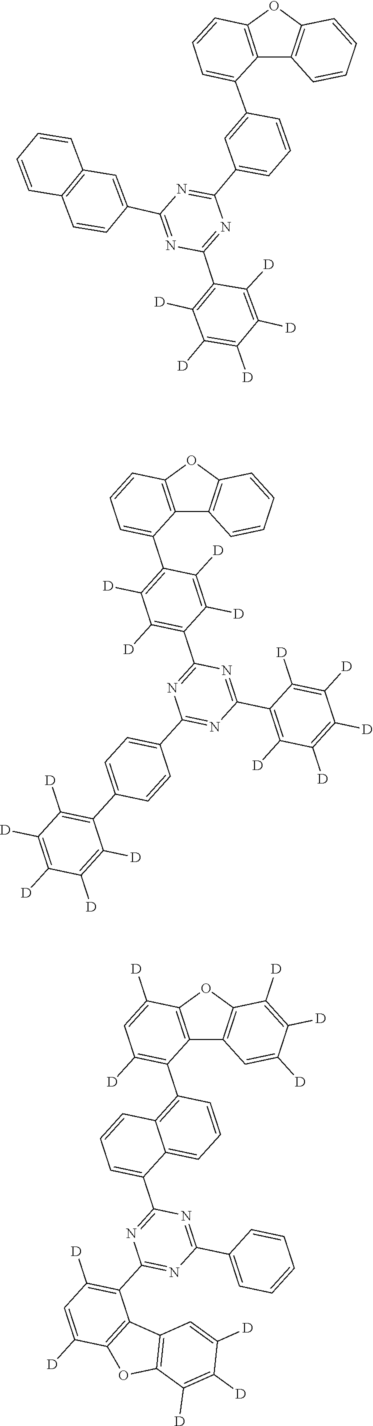 [SMILES: [2H]c1c([2H])c([2H])c(-c2nc(-c3cccc(-c4cccc5oc6ccccc6c45)c3)nc(-c3ccc4ccccc4c3)n2)c([2H])c1[2H] ; [2H]c1c([2H])c([2H])c(-c2ccc(-c3nc(-c4c([2H])c([2H])c([2H])c([2H])c4[2H])nc(-c4c([2H])c([2H])c(-c5cccc6oc7ccccc7c56)c([2H])c4[2H])n3)cc2)c([2H])c1[2H] ; [2H]c1cc2c(oc3c([2H])cc([2H])c(-c4nc(-c5ccccc5)nc(-c5cccc6c(-c7c([2H])cc([2H])c8oc9c([2H])c([2H])c([2H])cc9c78)cccc56)n4)c32)c([2H])c1[2H]]

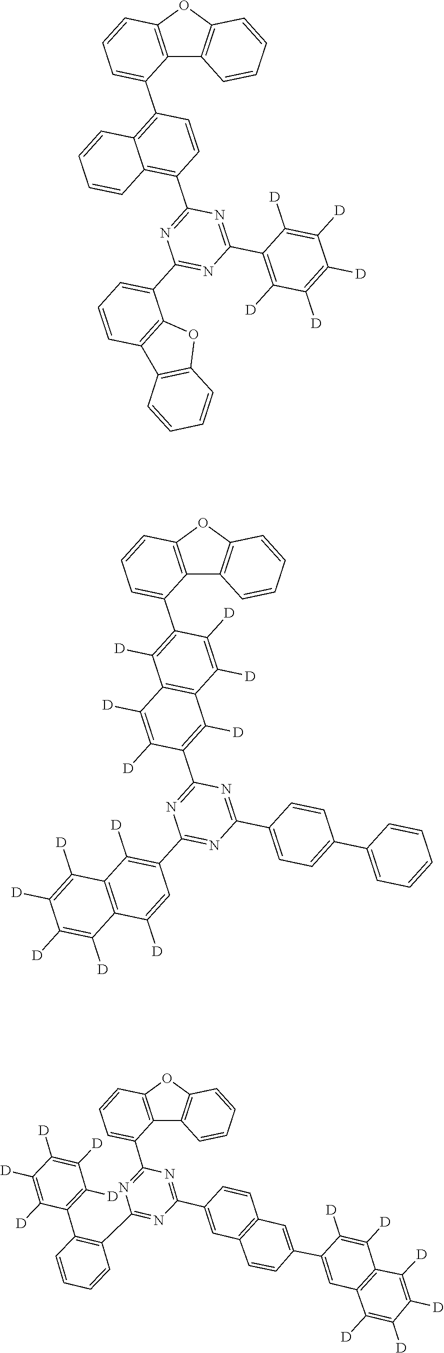 [SMILES: [2H]c1c([2H])c([2H])c(-c2nc(-c3ccc(-c4cccc5oc6ccccc6c45)c4ccccc34)nc(-c3cccc4c3oc3ccccc34)n2)c([2H])c1[2H] ; [2H]c1c([2H])c([2H])c2c([2H])c(-c3nc(-c4ccc(-c5ccccc5)cc4)nc(-c4c([2H])c([2H])c5c([2H])c(-c6cccc7oc8ccccc8c67)c([2H])c([2H])c5c4[2H])n3)cc([2H])c2c1[2H] ; [2H]c1c([2H])c([2H])c(-c2ccccc2-c2nc(-c3ccc4cc(-c5cc6c([2H])c([2H])c([2H])c([2H])c6c([2H])c5[2H])ccc4c3)nc(-c3cccc4oc5ccccc5c34)n2)c([2H])c1[2H]]

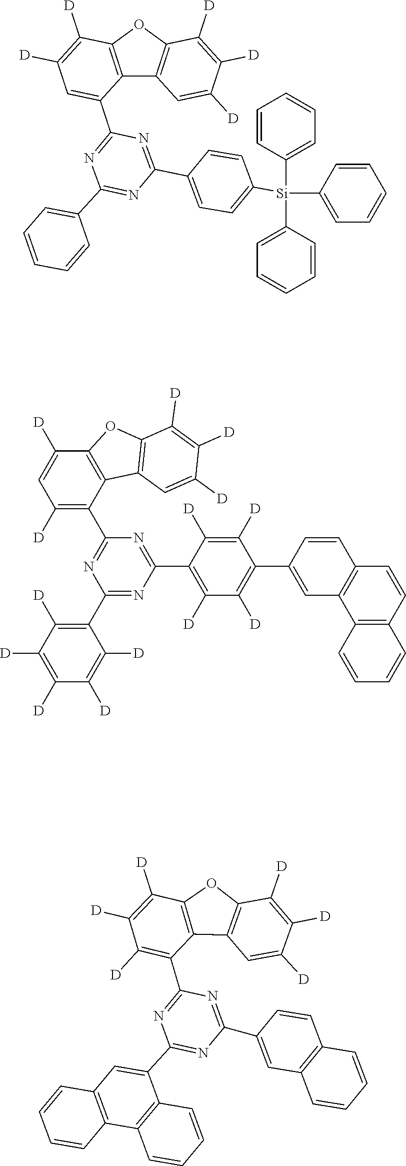 [SMILES: [2H]c1cc2c(oc3c([2H])c([2H])cc(-c4nc(-c5ccccc5)nc(-c5ccc([Si](c6ccccc6)(c6ccccc6)c6ccccc6)cc5)n4)c32)c([2H])c1[2H] ; [2H]c1cc2c(oc3c([2H])cc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c32)c([2H])c1[2H] ; [2H]c1cc2c(oc3c([2H])c([2H])c([2H])c(-c4nc(-c5ccc6ccccc6c5)nc(-c5cc6ccccc6c6ccccc56)n4)c32)c([2H])c1[2H]]

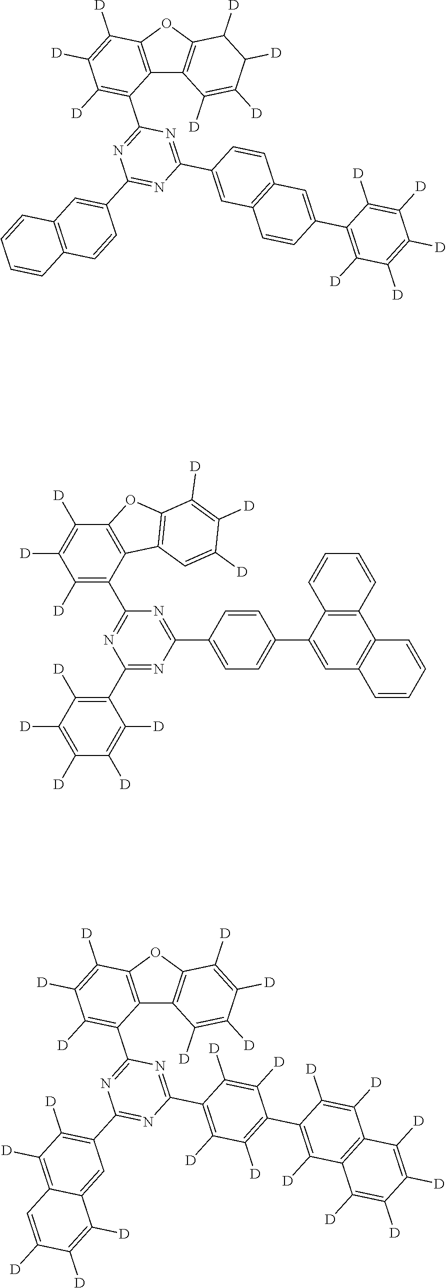 [SMILES: [2H]C1=C([2H])C([2H])C([2H])c2oc3c([2H])c([2H])c([2H])c(-c4nc(-c5ccc6ccccc6c5)nc(-c5ccc6cc(-c7c([2H])c([2H])c([2H])c([2H])c7[2H])ccc6c5)n4)c3c21 ; [2H]c1cc2c(oc3c([2H])c([2H])c([2H])c(-c4nc(-c5ccc(-c6cc7ccccc7c7ccccc67)cc5)nc(-c5c([2H])c([2H])c([2H])c([2H])c5[2H])n4)c32)c([2H])c1[2H] ; [2H]c1cc2c([2H])c([2H])c(-c3nc(-c4c([2H])c([2H])c(-c5c([2H])c([2H])c6c([2H])c([2H])c([2H])c([2H])c6c5[2H])c([2H])c4[2H])nc(-c4c([2H])c([2H])c([2H])c5oc6c([2H])c([2H])c([2H])c([2H])c6c45)n3)cc2c([2H])c1[2H]]

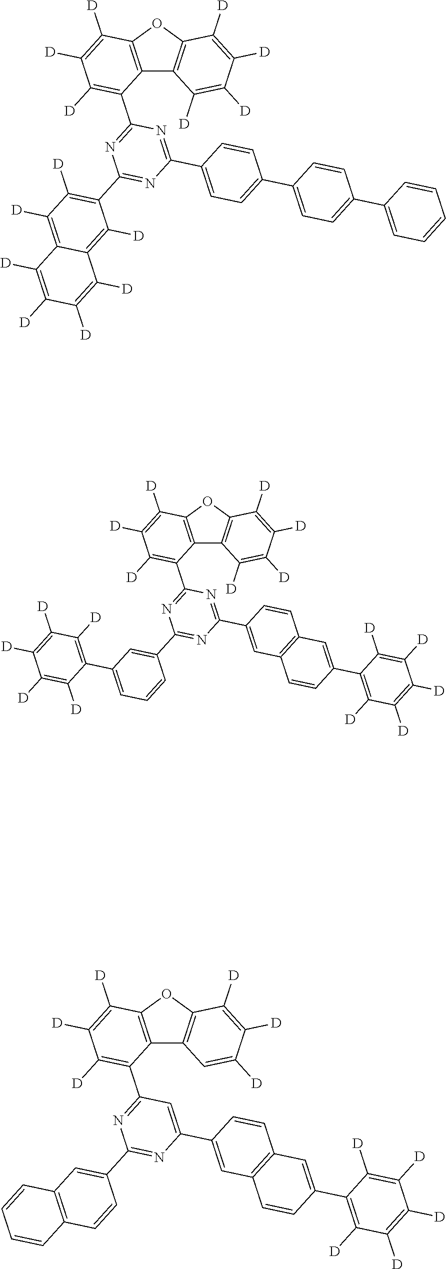 [SMILES: [2H]c1c([2H])c([2H])c2c(oc3c([2H])c([2H])c([2H])c(-c4nc(-c5ccc(-c6ccc(-c7ccccc7)cc6)cc5)nc(-c5c([2H])c([2H])c6c([2H])c([2H])c([2H])c([2H])c6c5[2H])n4)c32)c1[2H] ; [2H]c1c([2H])c([2H])c(-c2cccc(-c3nc(-c4ccc5cc(-c6c([2H])c([2H])c([2H])c([2H])c6[2H])ccc5c4)nc(-c4c([2H])c([2H])c([2H])c5oc6c([2H])c([2H])c([2H])c([2H])c6c45)n3)c2)c([2H])c1[2H] ; [2H]c1cc2c(oc3c([2H])c([2H])c([2H])c(-c4cc(-c5ccc6cc(-c7c([2H])c([2H])c([2H])c([2H])c7[2H])ccc6c5)nc(-c5ccc6ccccc6c5)n4)c32)c([2H])c1[2H]]

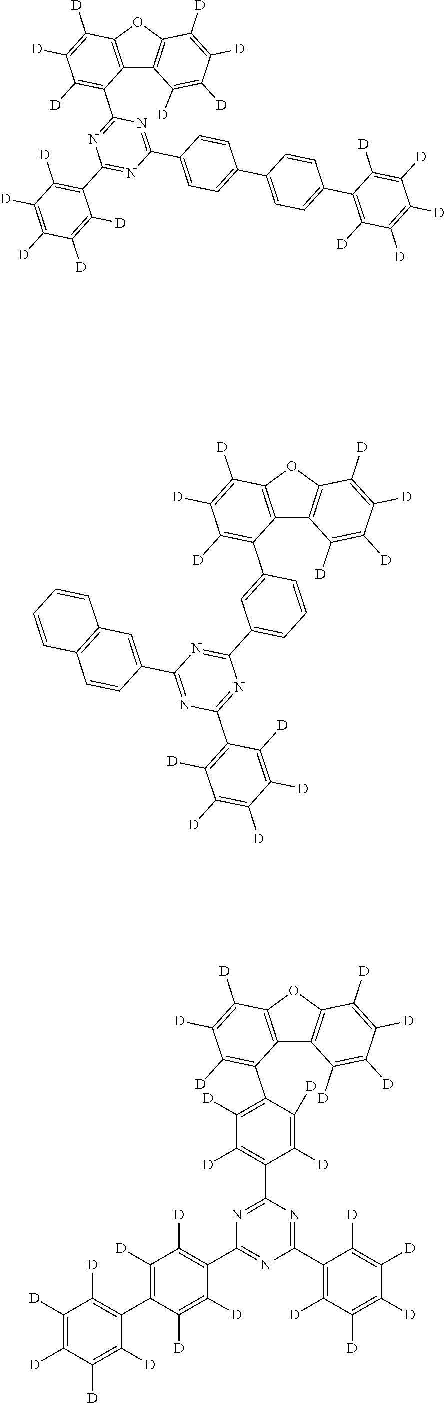

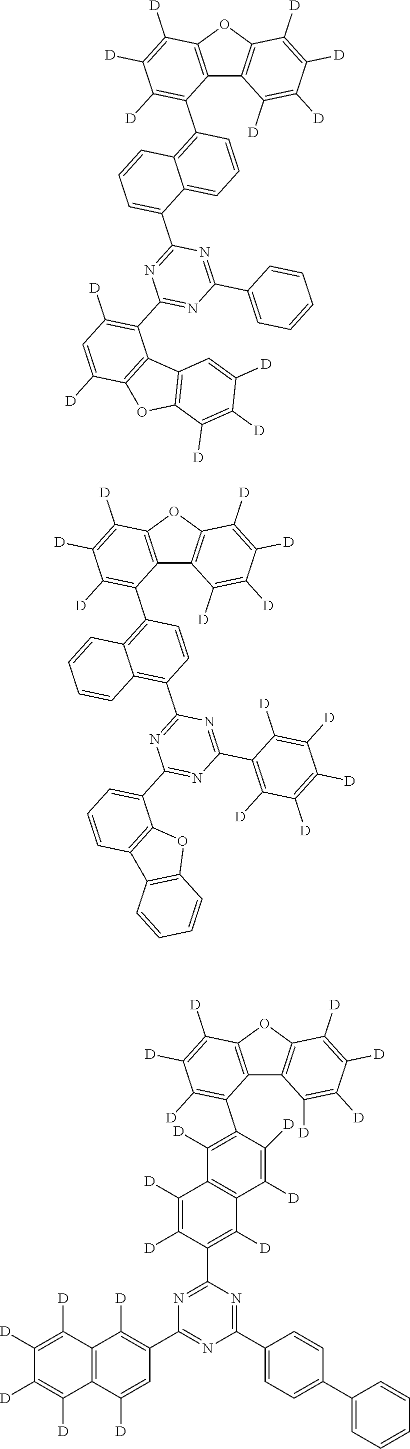 [SMILES: [2H]c1cc2c(oc3c([2H])cc([2H])c(-c4nc(-c5ccccc5)nc(-c5cccc6c(-c7c([2H])c([2H])c([2H])c8oc9c([2H])c([2H])c([2H])c([2H])c9c78)cccc56)n4)c32)c([2H])c1[2H] ; [2H]c1c([2H])c([2H])c(-c2nc(-c3ccc(-c4c([2H])c([2H])c([2H])c5oc6c([2H])c([2H])c([2H])c([2H])c6c45)c4ccccc34)nc(-c3cccc4c3oc3ccccc34)n2)c([2H])c1[2H] ; [2H]c1c([2H])c([2H])c2c(oc3c([2H])c([2H])c([2H])c(-c4c([2H])c([2H])c5c([2H])c(-c6nc(-c7ccc(-c8ccccc8)cc7)nc(-c7cc([2H])c8c([2H])c([2H])c([2H])c([2H])c8c7[2H])n6)c([2H])c([2H])c5c4[2H])c32)c1[2H]]

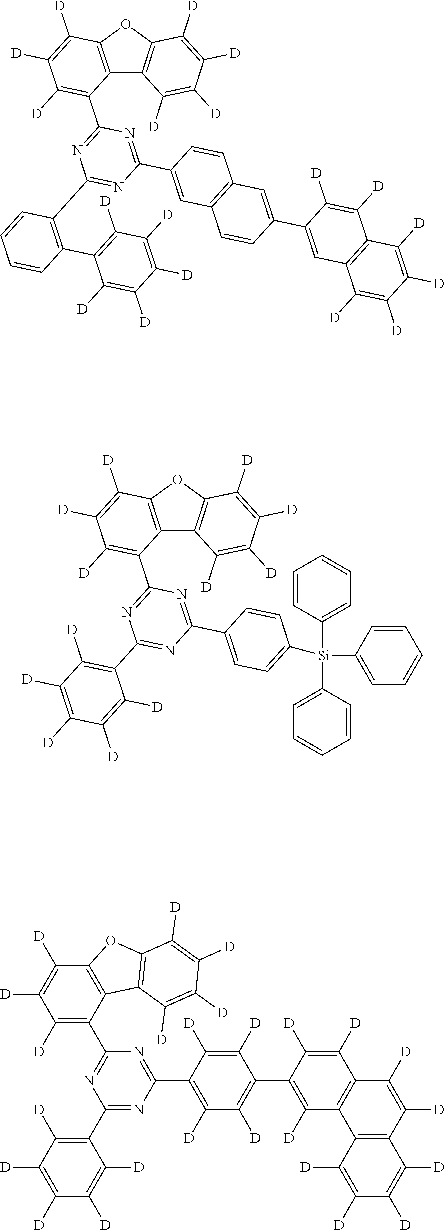

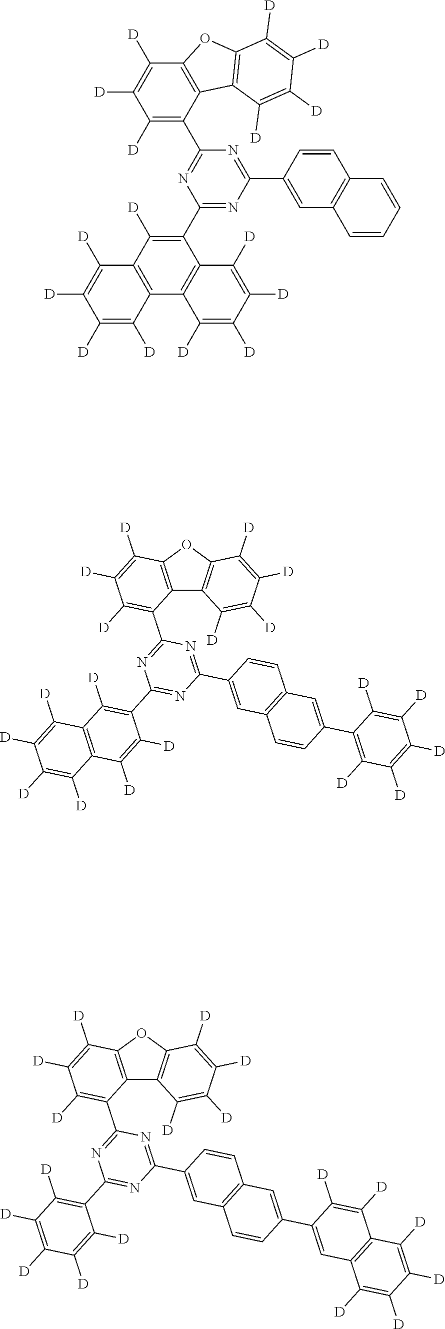

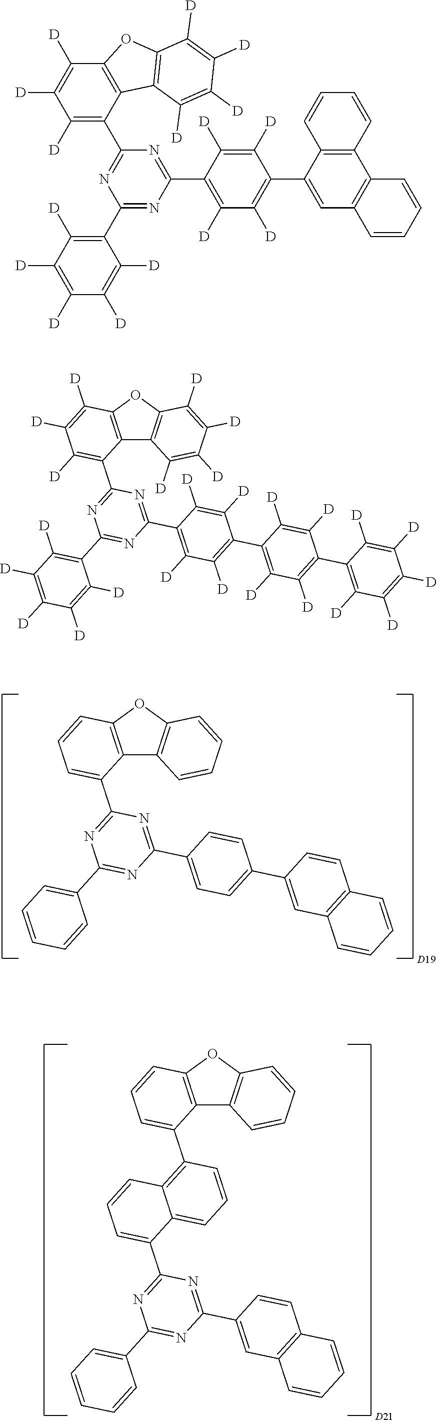

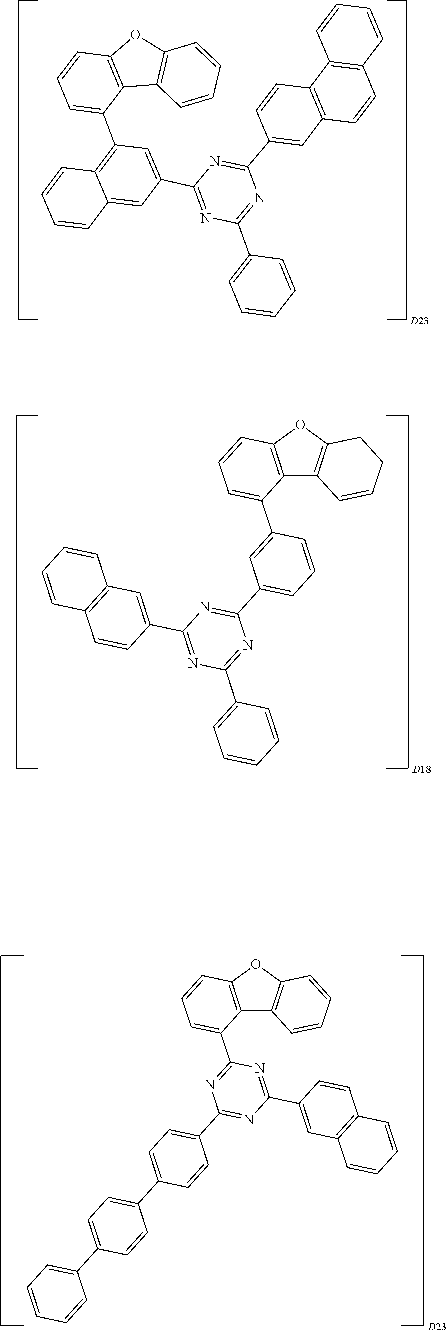

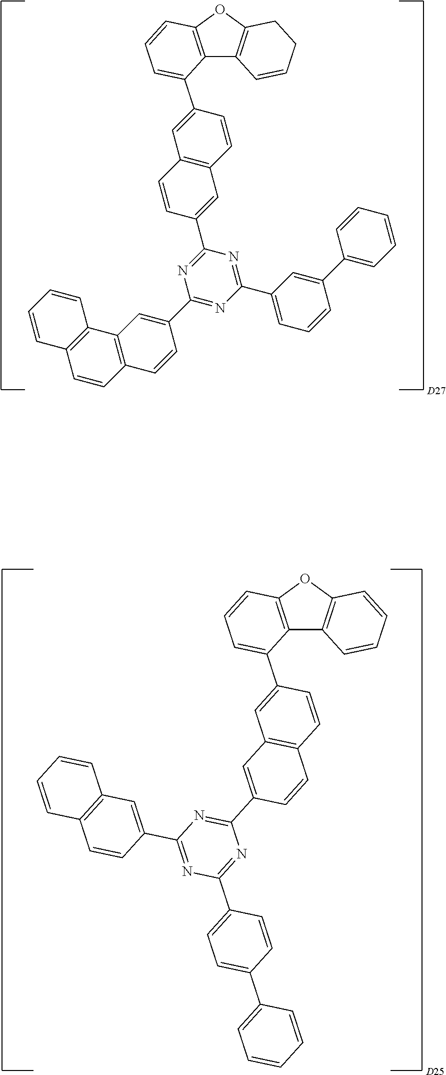

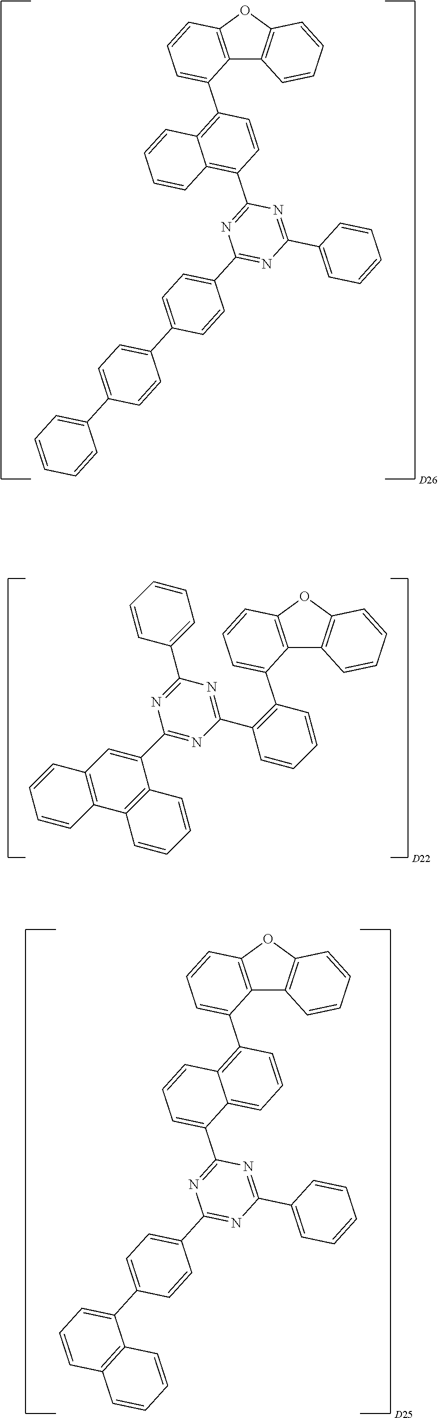

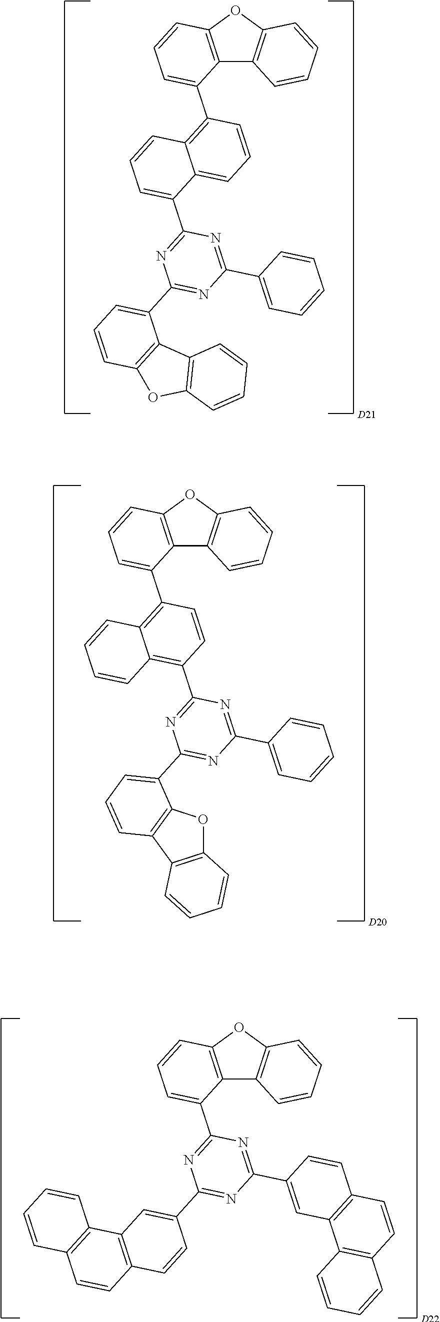

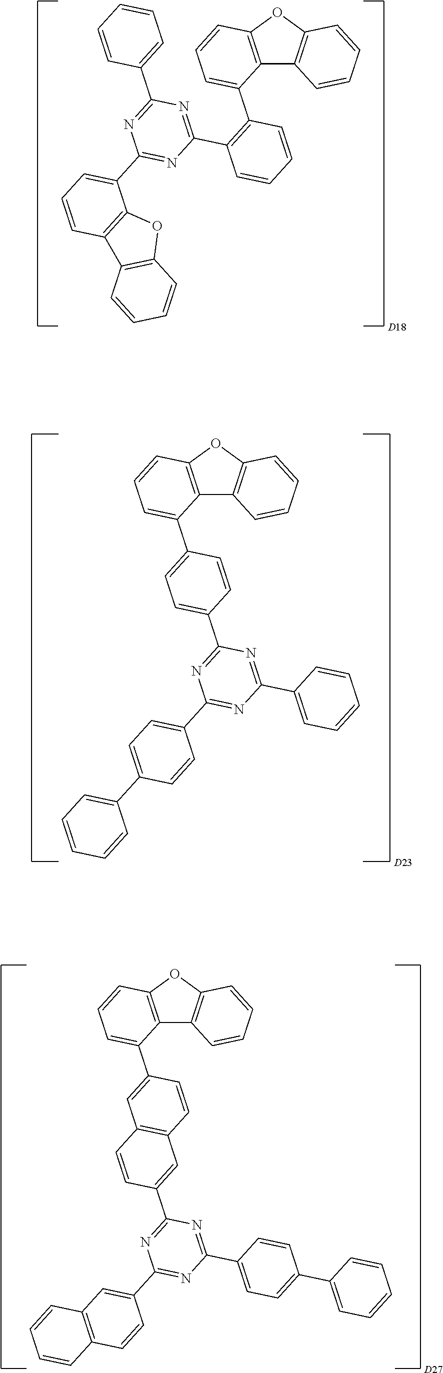

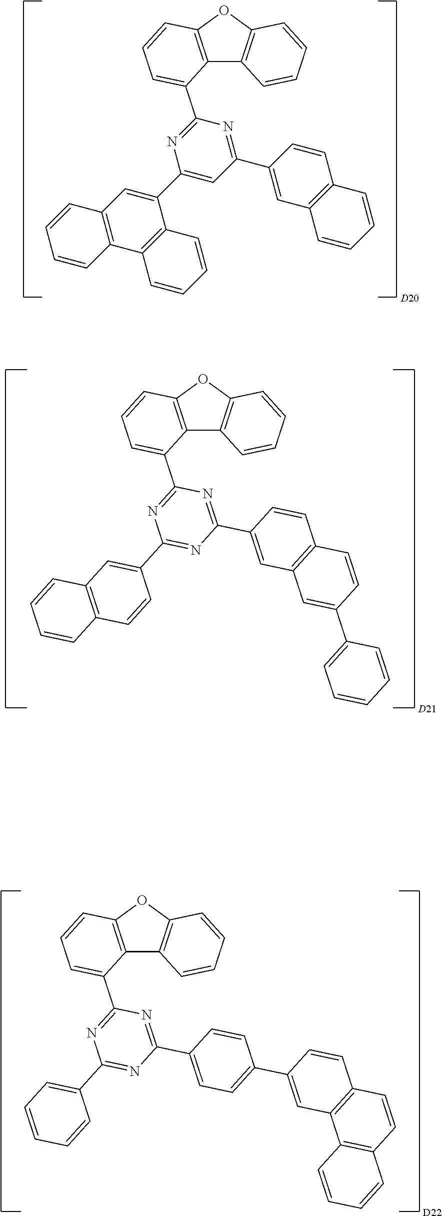

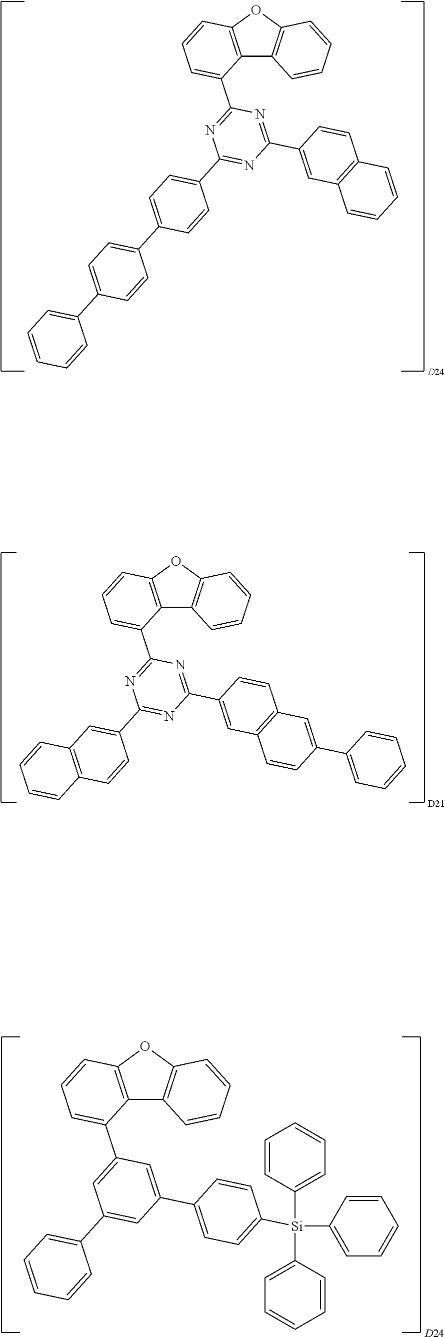

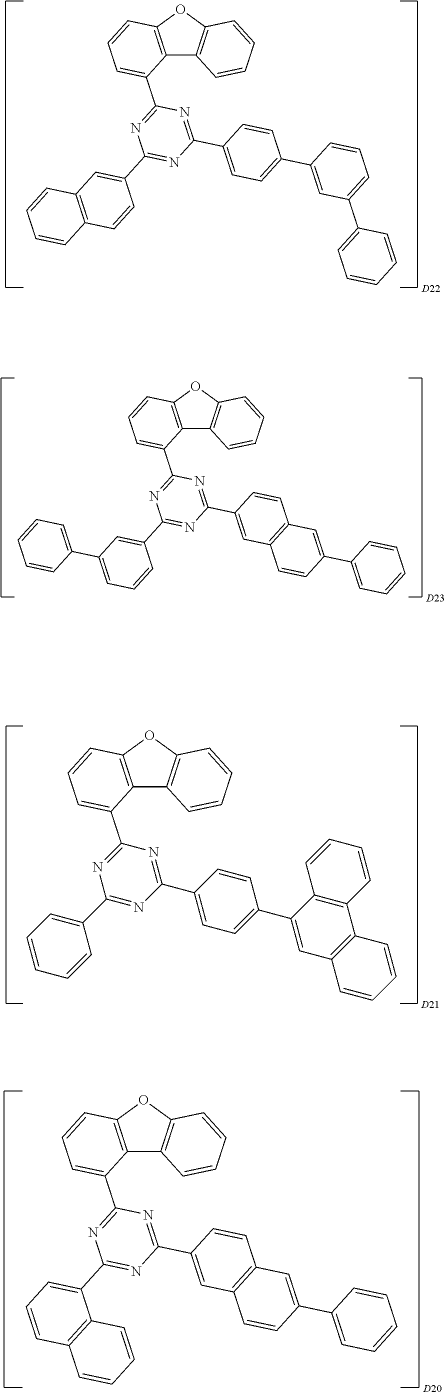

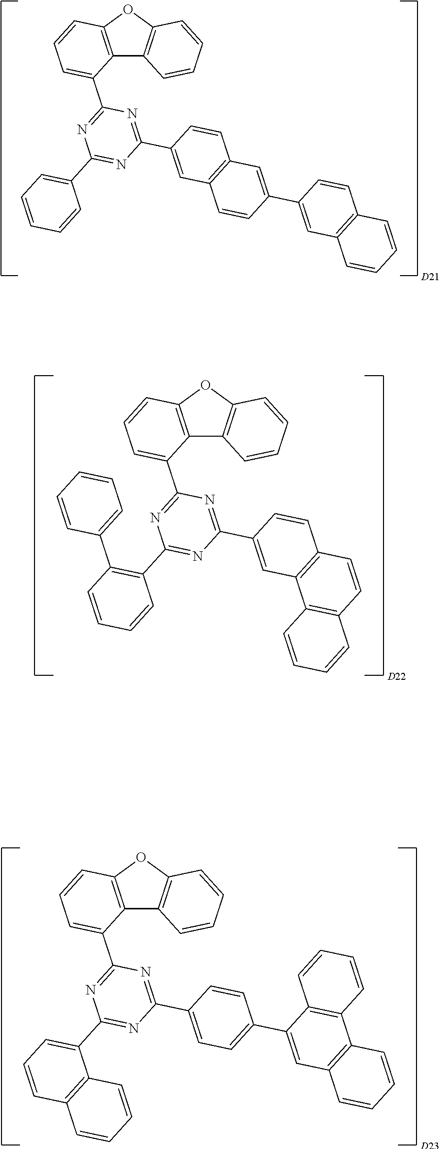

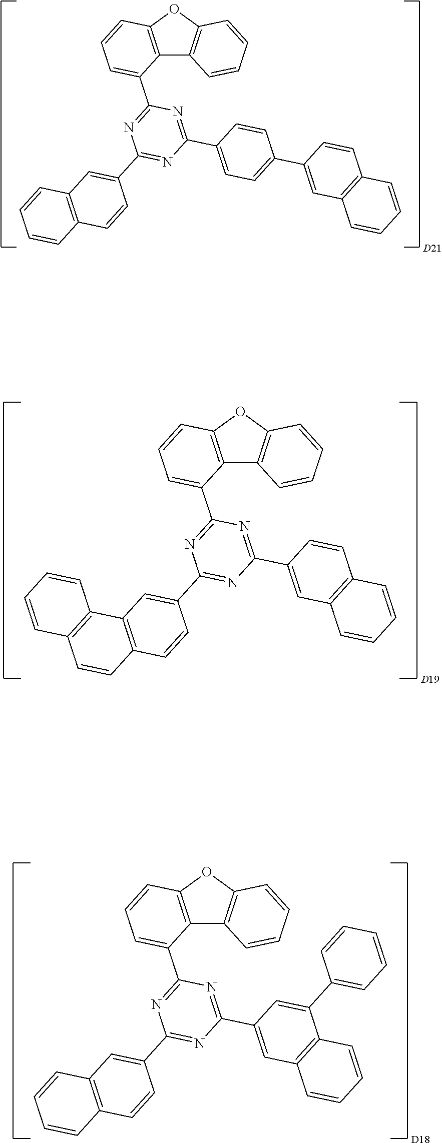

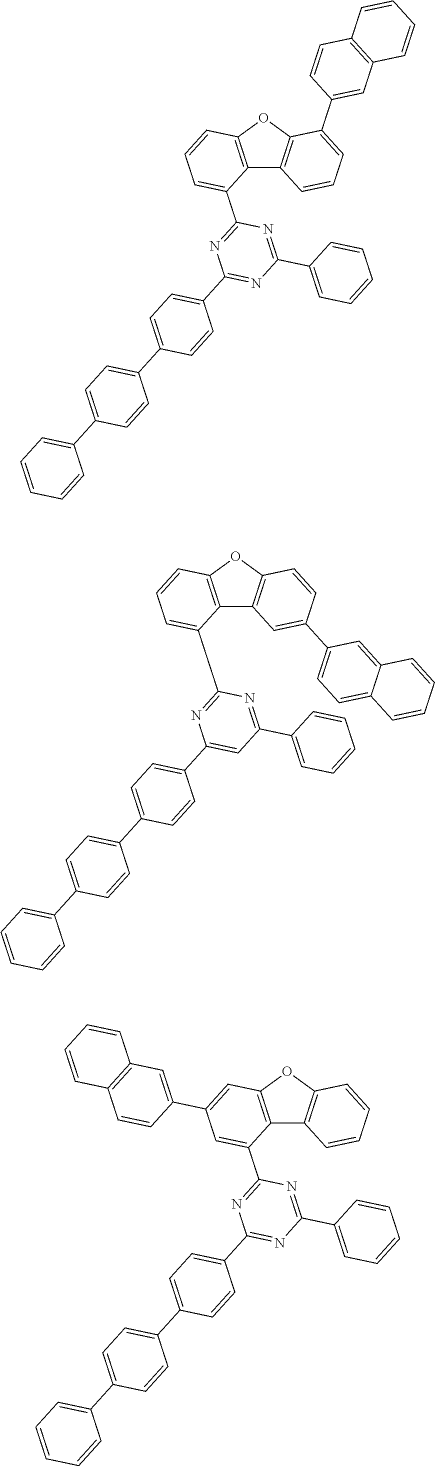

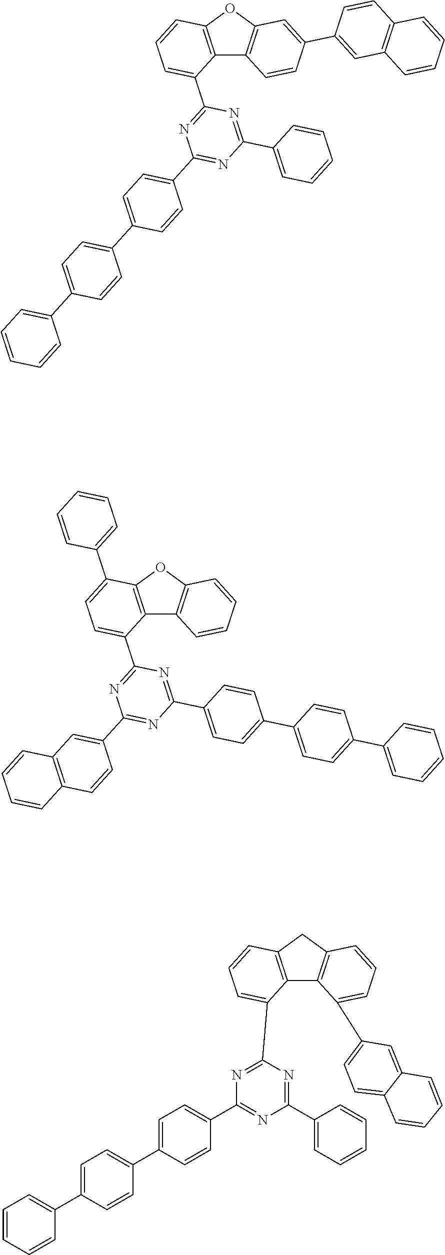

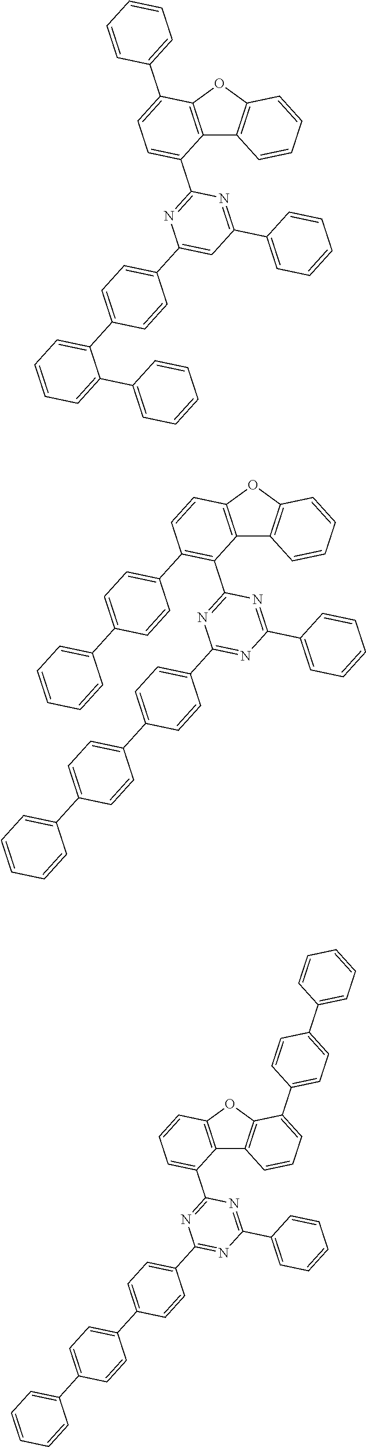

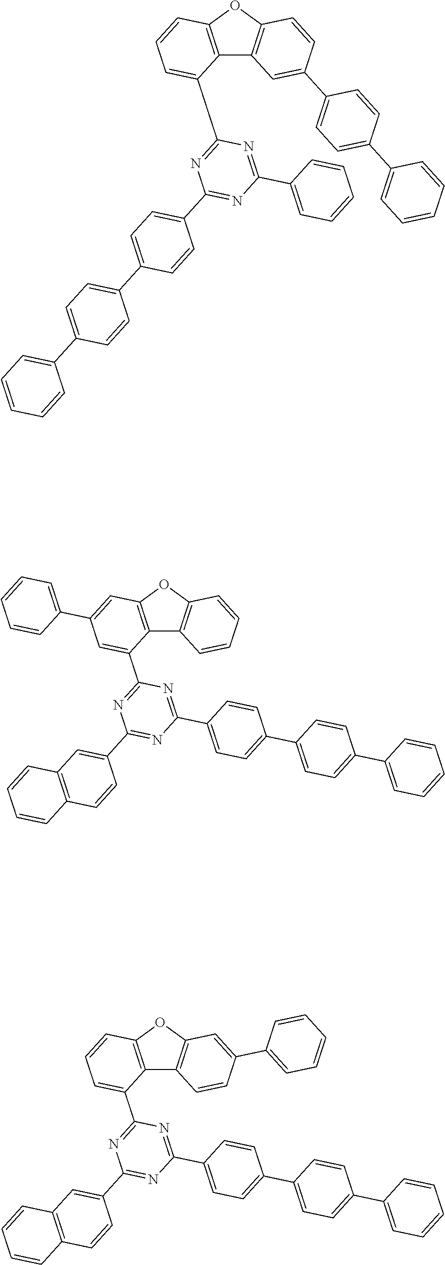

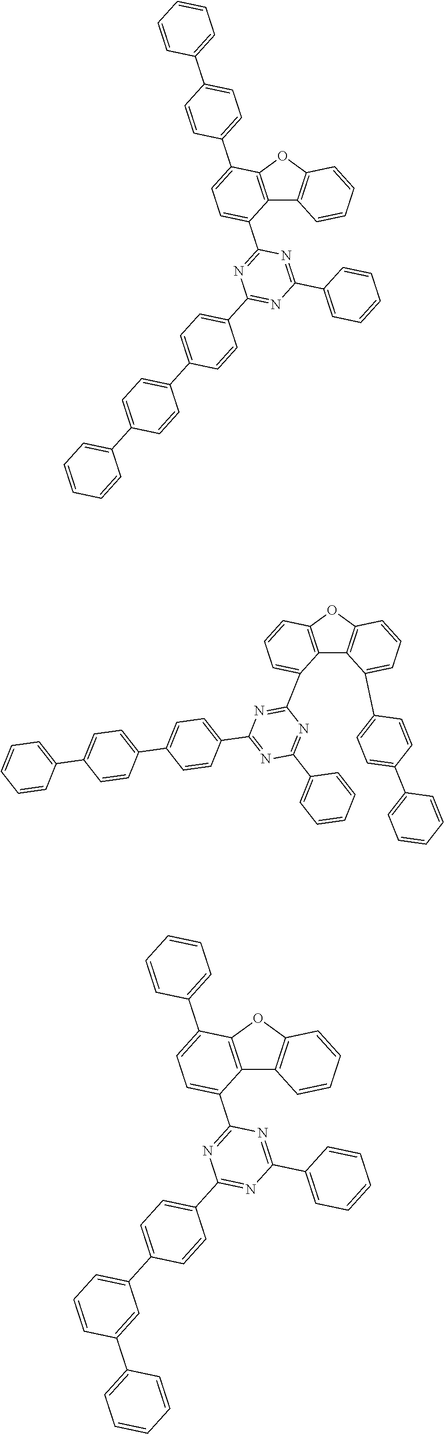 [SMILES: c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1 ; c1ccc(-c2cccc(-c3ccc(-c4nc(-c5ccccc5)nc(-c5ccc(-c6ccccc6)c6oc7ccccc7c56)n4)cc3)c2)cc1]

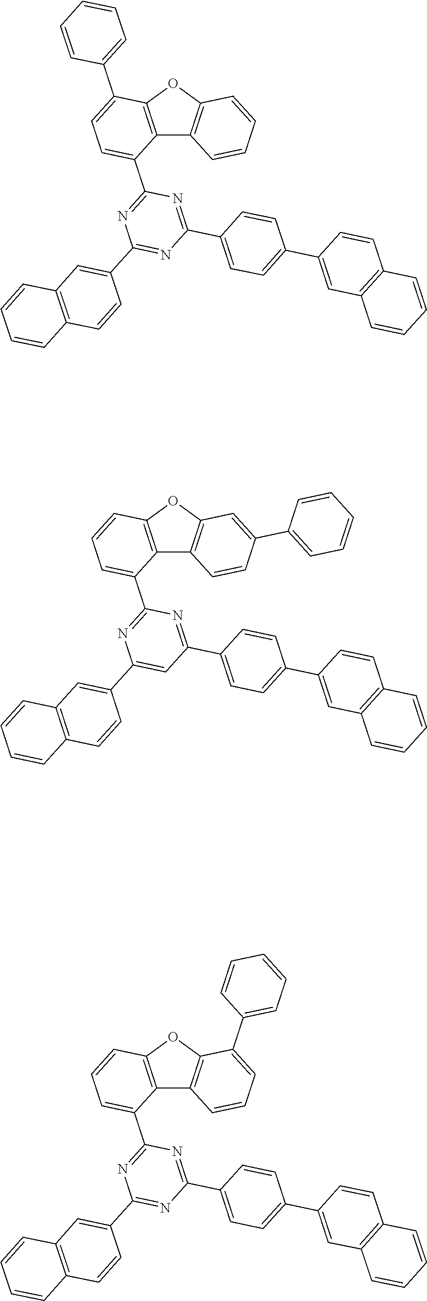 [SMILES: c1ccc(-c2ccc(-c3nc(-c4ccc(-c5ccc6ccccc6c5)cc4)nc(-c4ccc5ccccc5c4)n3)c3c2oc2ccccc23)cc1 ; c1ccc(-c2ccc3c(c2)oc2cccc(-c4nc(-c5ccc(-c6ccc7ccccc7c6)cc5)cc(-c5ccc6ccccc6c5)n4)c23)cc1 ; c1ccc(-c2cccc3c2oc2cccc(-c4nc(-c5ccc(-c6ccc7ccccc7c6)cc5)nc(-c5ccc6ccccc6c5)n4)c23)cc1]

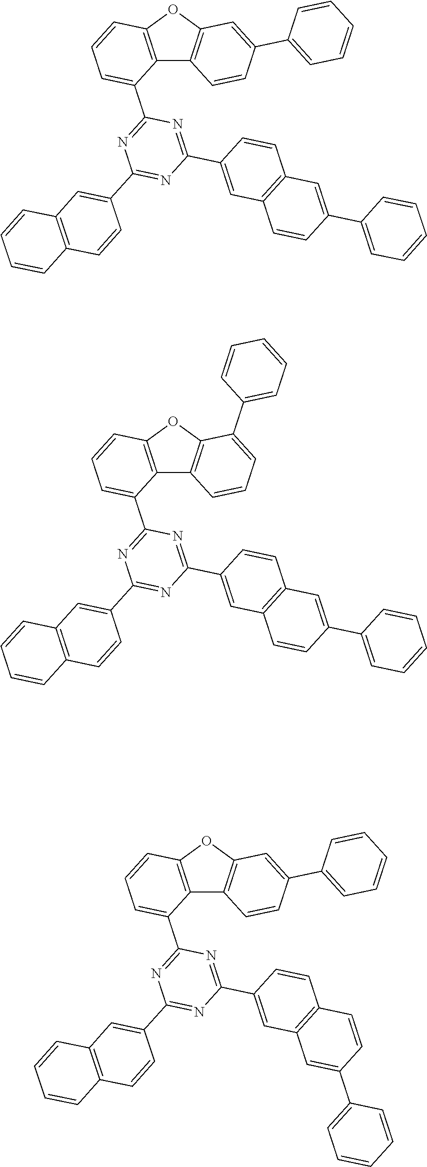 [SMILES: c1ccc(-c2ccc3cc(-c4nc(-c5ccc6ccccc6c5)nc(-c5cccc6oc7cc(-c8ccccc8)ccc7c56)n4)ccc3c2)cc1 ; c1ccc(-c2ccc3cc(-c4nc(-c5ccc6ccccc6c5)nc(-c5cccc6oc7c(-c8ccccc8)cccc7c56)n4)ccc3c2)cc1 ; c1ccc(-c2ccc3ccc(-c4nc(-c5ccc6ccccc6c5)nc(-c5cccc6oc7cc(-c8ccccc8)ccc7c56)n4)cc3c2)cc1]

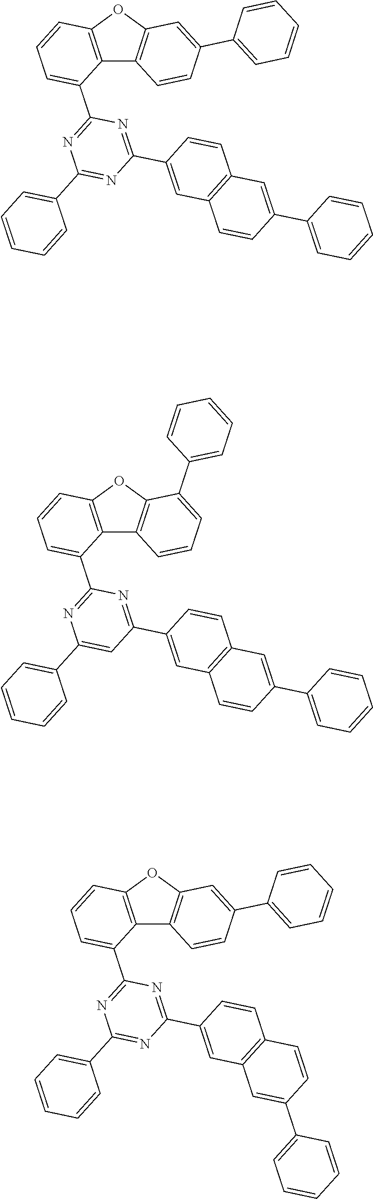 [SMILES: c1ccc(-c2ccc3cc(-c4nc(-c5ccccc5)nc(-c5cccc6oc7cc(-c8ccccc8)ccc7c56)n4)ccc3c2)cc1 ; c1ccc(-c2ccc3cc(-c4cc(-c5ccccc5)nc(-c5cccc6oc7c(-c8ccccc8)cccc7c56)n4)ccc3c2)cc1 ; c1ccc(-c2ccc3ccc(-c4nc(-c5ccccc5)nc(-c5cccc6oc7cc(-c8ccccc8)ccc7c56)n4)cc3c2)cc1]

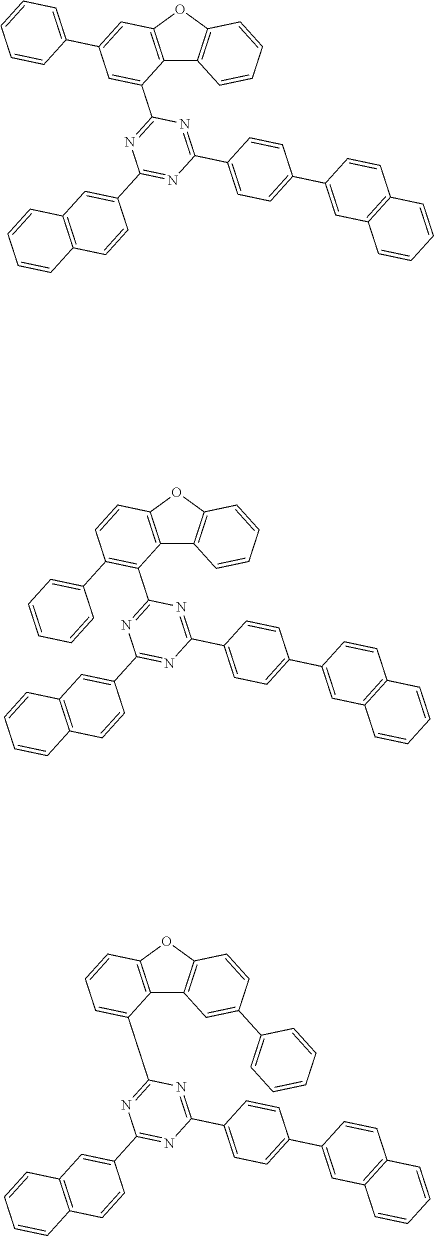 [SMILES: c1ccc(-c2cc(-c3nc(-c4ccc(-c5ccc6ccccc6c5)cc4)nc(-c4ccc5ccccc5c4)n3)c3c(c2)oc2ccccc23)cc1 ; c1ccc(-c2ccc3oc4ccccc4c3c2-c2nc(-c3ccc(-c4ccc5ccccc5c4)cc3)nc(-c3ccc4ccccc4c3)n2)cc1 ; c1ccc(-c2ccc3oc4cccc(-c5nc(-c6ccc(-c7ccc8ccccc8c7)cc6)nc(-c6ccc7ccccc7c6)n5)c4c3c2)cc1]

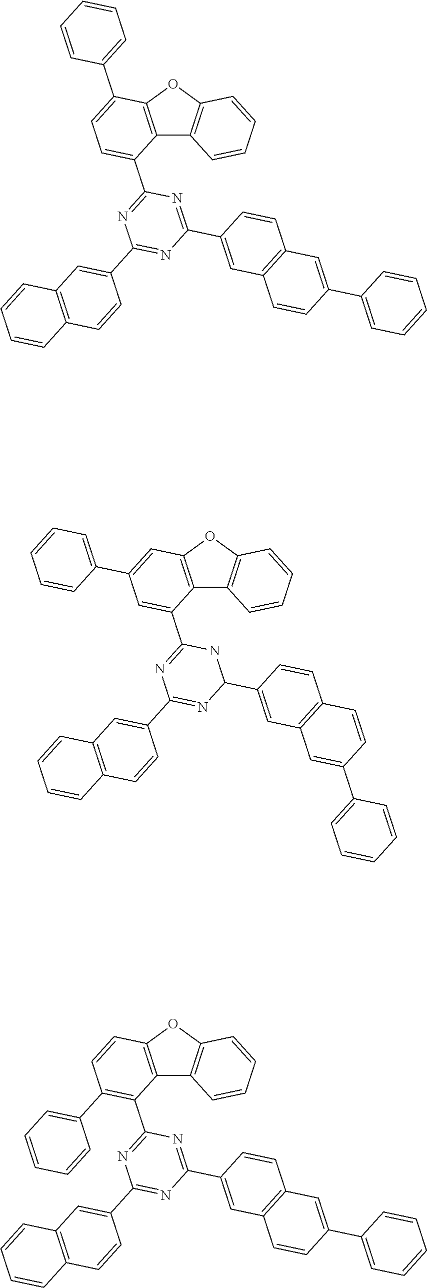

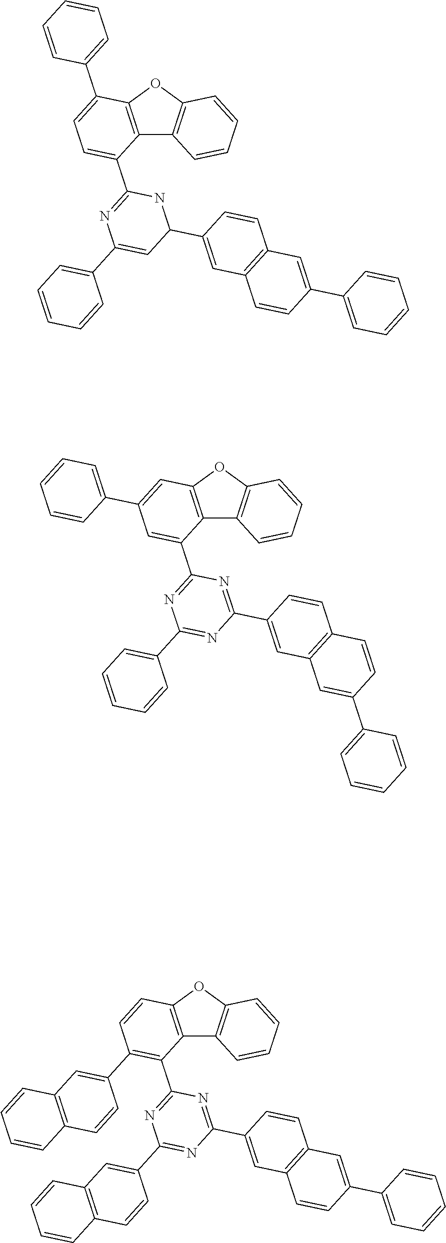

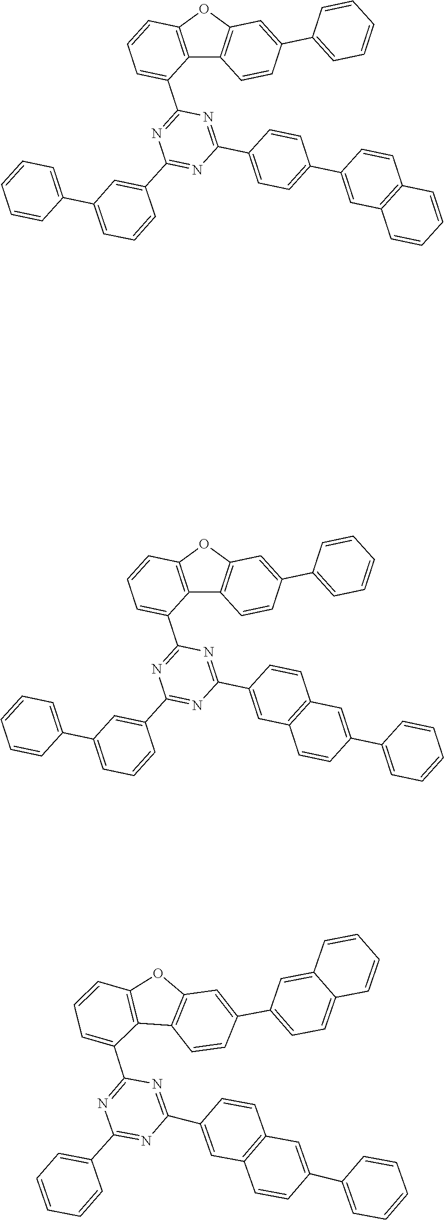 [SMILES: c1ccc(-c2cccc(-c3nc(-c4ccc(-c5ccc6ccccc6c5)cc4)nc(-c4cccc5oc6cc(-c7ccccc7)ccc6c45)n3)c2)cc1 ; c1ccc(-c2cccc(-c3nc(-c4ccc5cc(-c6ccccc6)ccc5c4)nc(-c4cccc5oc6cc(-c7ccccc7)ccc6c45)n3)c2)cc1 ; c1ccc(-c2ccc3cc(-c4nc(-c5ccccc5)nc(-c5cccc6oc7cc(-c8ccc9ccccc9c8)ccc7c56)n4)ccc3c2)cc1]

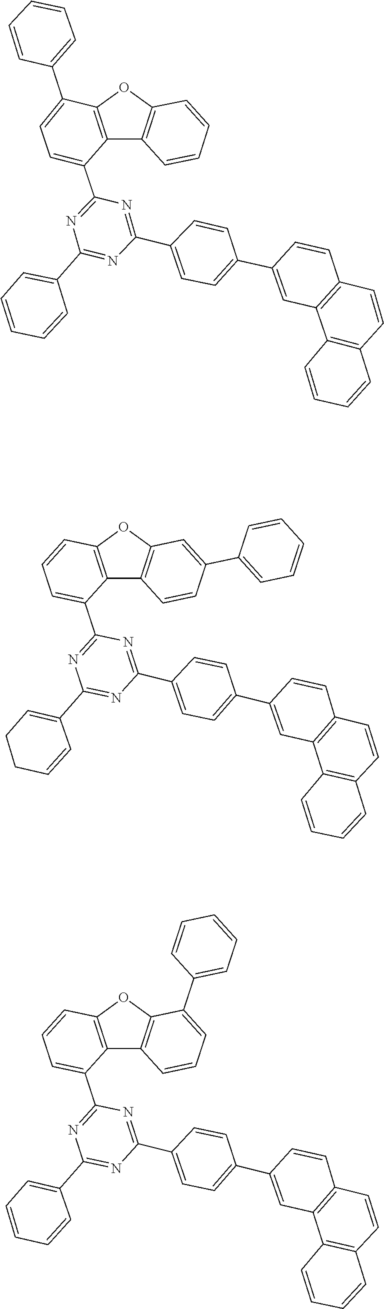 [SMILES: c1ccc(-c2nc(-c3ccc(-c4ccc5ccc6ccccc6c5c4)cc3)nc(-c3ccc(-c4ccccc4)c4oc5ccccc5c34)n2)cc1 ; C1=CC(c2nc(-c3ccc(-c4ccc5ccc6ccccc6c5c4)cc3)nc(-c3cccc4oc5cc(-c6ccccc6)ccc5c34)n2)=CCC1 ; c1ccc(-c2nc(-c3ccc(-c4ccc5ccc6ccccc6c5c4)cc3)nc(-c3cccc4oc5c(-c6ccccc6)cccc5c34)n2)cc1]

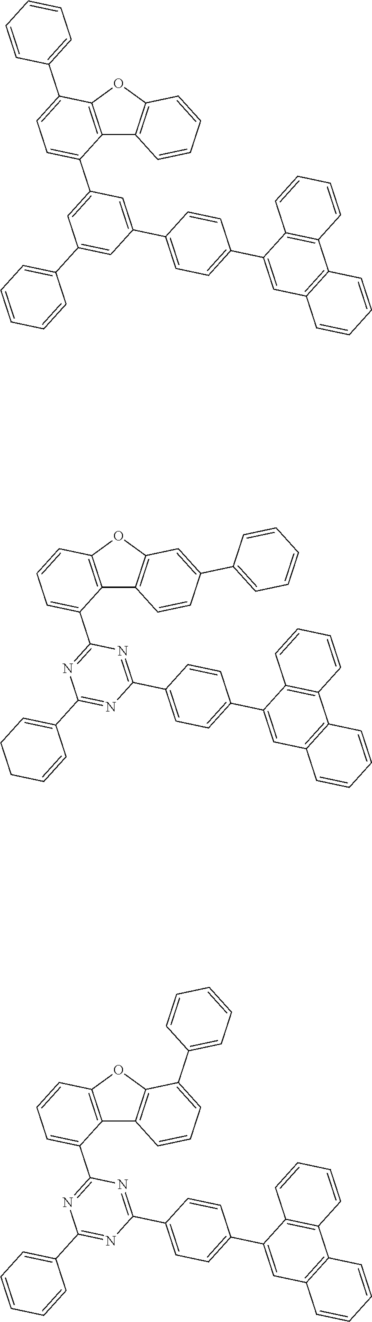 [SMILES: c1ccc(-c2cc(-c3ccc(-c4cc5ccccc5c5ccccc45)cc3)cc(-c3ccc(-c4ccccc4)c4oc5ccccc5c34)c2)cc1 ; C1=CC(c2nc(-c3ccc(-c4cc5ccccc5c5ccccc45)cc3)nc(-c3cccc4oc5cc(-c6ccccc6)ccc5c34)n2)=CCC1 ; c1ccc(-c2nc(-c3ccc(-c4cc5ccccc5c5ccccc45)cc3)nc(-c3cccc4oc5c(-c6ccccc6)cccc5c34)n2)cc1]

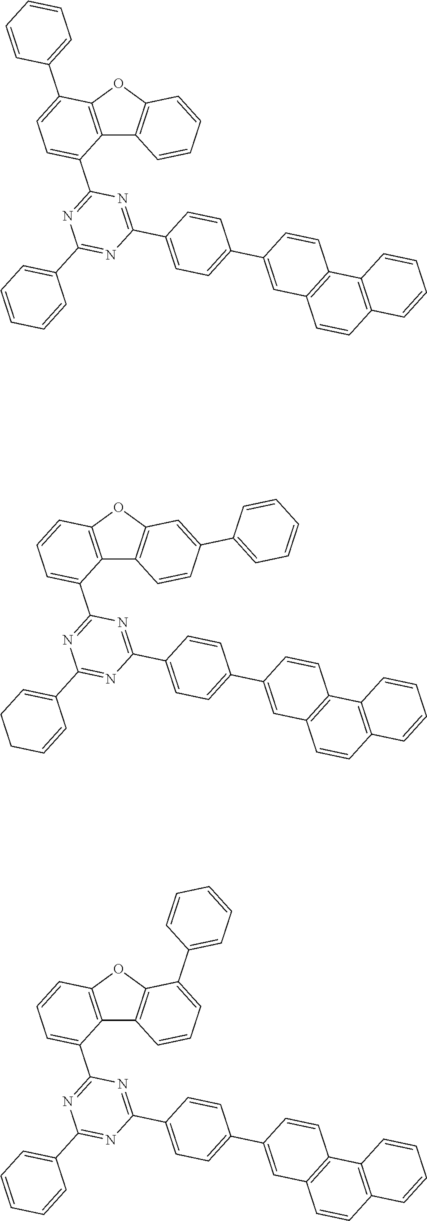 [SMILES: c1ccc(-c2nc(-c3ccc(-c4ccc5c(ccc6ccccc65)c4)cc3)nc(-c3ccc(-c4ccccc4)c4oc5ccccc5c34)n2)cc1 ; C1=CC(c2nc(-c3ccc(-c4ccc5c(ccc6ccccc65)c4)cc3)nc(-c3cccc4oc5cc(-c6ccccc6)ccc5c34)n2)=CCC1 ; c1ccc(-c2nc(-c3ccc(-c4ccc5c(ccc6ccccc65)c4)cc3)nc(-c3cccc4oc5c(-c6ccccc6)cccc5c34)n2)cc1]

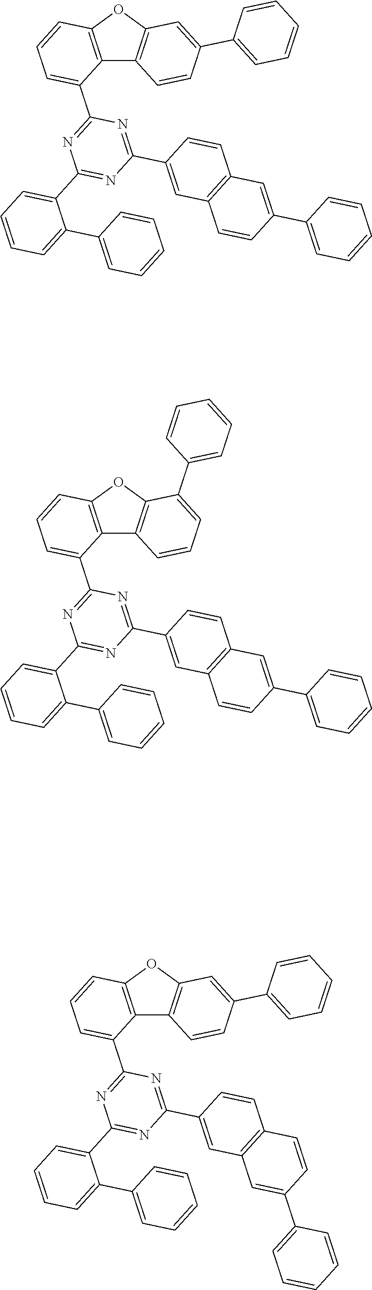 [SMILES: c1ccc(-c2ccc3cc(-c4nc(-c5ccccc5-c5ccccc5)nc(-c5cccc6oc7cc(-c8ccccc8)ccc7c56)n4)ccc3c2)cc1 ; c1ccc(-c2ccc3cc(-c4nc(-c5ccccc5-c5ccccc5)nc(-c5cccc6oc7c(-c8ccccc8)cccc7c56)n4)ccc3c2)cc1 ; c1ccc(-c2ccc3ccc(-c4nc(-c5ccccc5-c5ccccc5)nc(-c5cccc6oc7cc(-c8ccccc8)ccc7c56)n4)cc3c2)cc1]

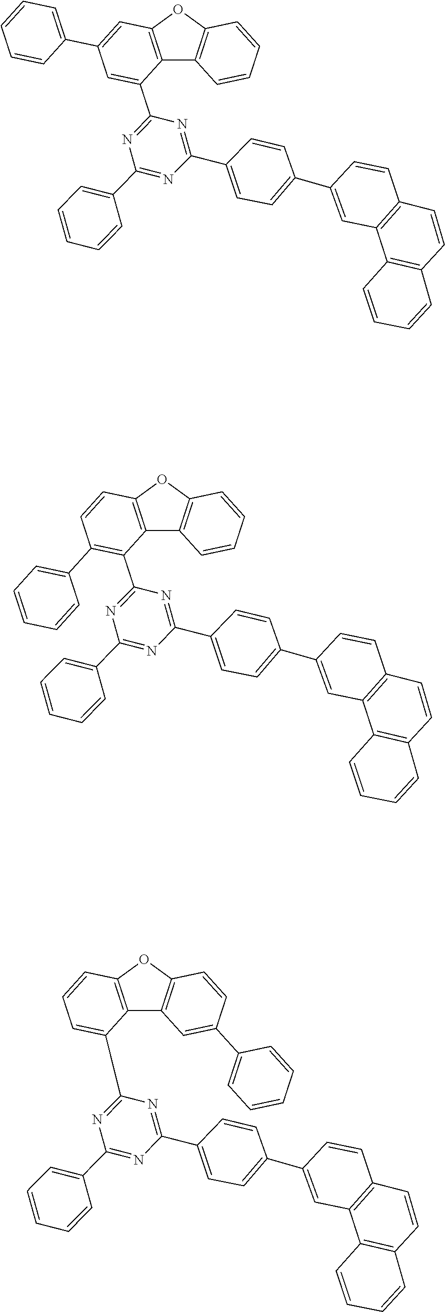 [SMILES: c1ccc(-c2cc(-c3nc(-c4ccccc4)nc(-c4ccc(-c5ccc6ccc7ccccc7c6c5)cc4)n3)c3c(c2)oc2ccccc23)cc1 ; c1ccc(-c2nc(-c3ccc(-c4ccc5ccc6ccccc6c5c4)cc3)nc(-c3c(-c4ccccc4)ccc4oc5ccccc5c34)n2)cc1 ; c1ccc(-c2ccc3oc4cccc(-c5nc(-c6ccccc6)nc(-c6ccc(-c7ccc8ccc9ccccc9c8c7)cc6)n5)c4c3c2)cc1]

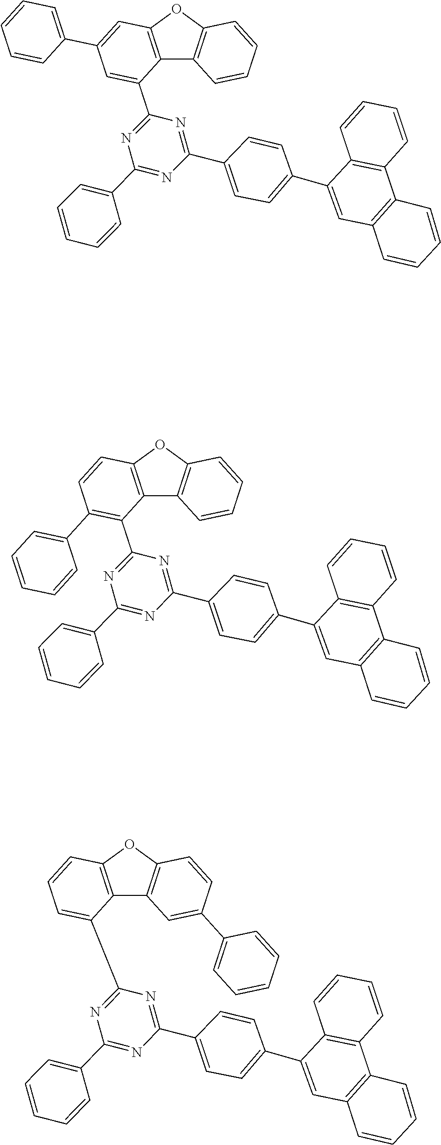 [SMILES: c1ccc(-c2cc(-c3nc(-c4ccccc4)nc(-c4ccc(-c5cc6ccccc6c6ccccc56)cc4)n3)c3c(c2)oc2ccccc23)cc1 ; c1ccc(-c2nc(-c3ccc(-c4cc5ccccc5c5ccccc45)cc3)nc(-c3c(-c4ccccc4)ccc4oc5ccccc5c34)n2)cc1 ; c1ccc(-c2ccc3oc4cccc(-c5nc(-c6ccccc6)nc(-c6ccc(-c7cc8ccccc8c8ccccc78)cc6)n5)c4c3c2)cc1]

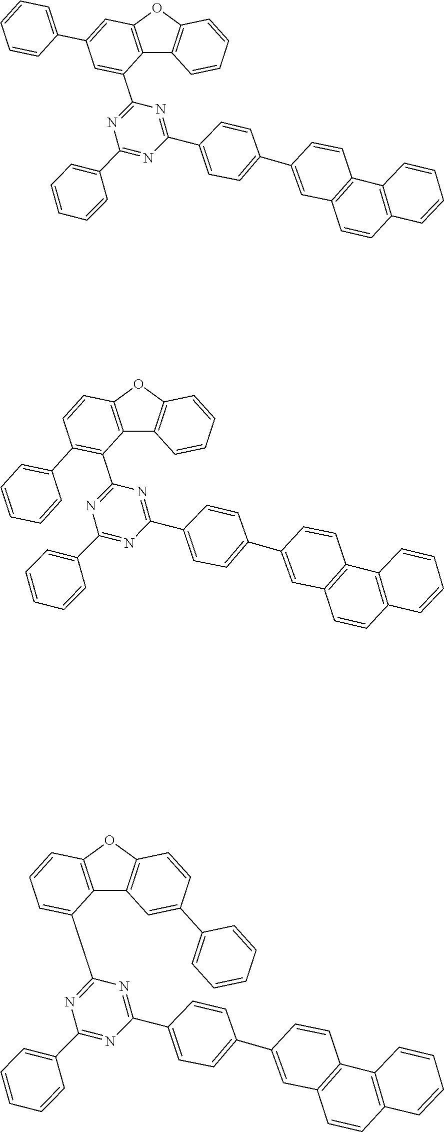 [SMILES: c1ccc(-c2cc(-c3nc(-c4ccccc4)nc(-c4ccc(-c5ccc6c(ccc7ccccc76)c5)cc4)n3)c3c(c2)oc2ccccc23)cc1 ; c1ccc(-c2nc(-c3ccc(-c4ccc5c(ccc6ccccc65)c4)cc3)nc(-c3c(-c4ccccc4)ccc4oc5ccccc5c34)n2)cc1 ; c1ccc(-c2ccc3oc4cccc(-c5nc(-c6ccccc6)nc(-c6ccc(-c7ccc8c(ccc9ccccc98)c7)cc6)n5)c4c3c2)cc1]

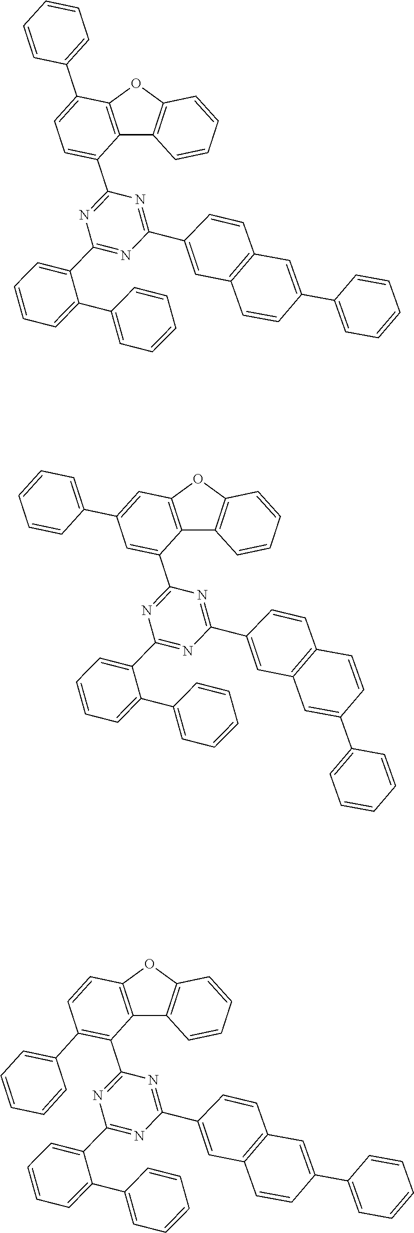 [SMILES: c1ccc(-c2ccc3cc(-c4nc(-c5ccccc5-c5ccccc5)nc(-c5ccc(-c6ccccc6)c6oc7ccccc7c56)n4)ccc3c2)cc1 ; c1ccc(-c2ccc3ccc(-c4nc(-c5ccccc5-c5ccccc5)nc(-c5cc(-c6ccccc6)cc6oc7ccccc7c56)n4)cc3c2)cc1 ; c1ccc(-c2ccc3cc(-c4nc(-c5ccccc5-c5ccccc5)nc(-c5c(-c6ccccc6)ccc6oc7ccccc7c56)n4)ccc3c2)cc1]

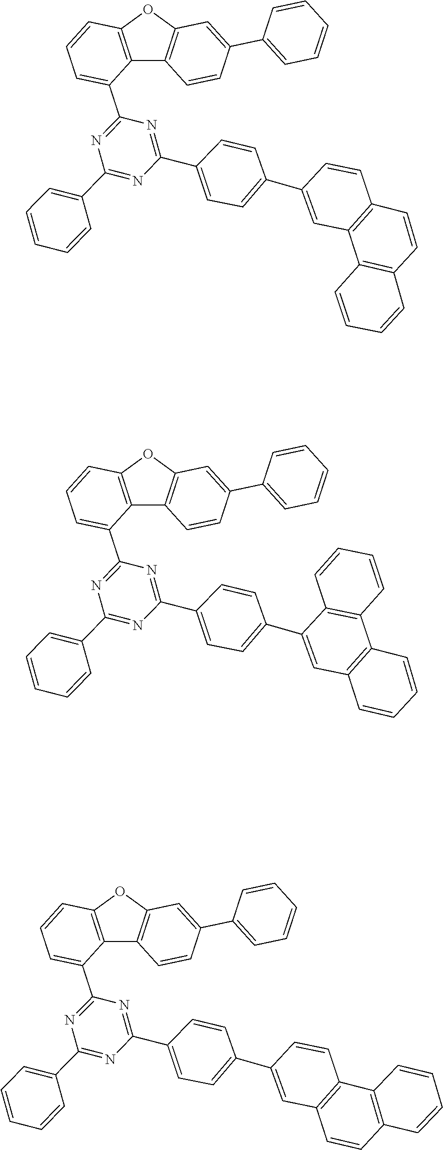 [SMILES: c1ccc(-c2ccc3c(c2)oc2cccc(-c4nc(-c5ccccc5)nc(-c5ccc(-c6ccc7ccc8ccccc8c7c6)cc5)n4)c23)cc1 ; c1ccc(-c2ccc3c(c2)oc2cccc(-c4nc(-c5ccccc5)nc(-c5ccc(-c6cc7ccccc7c7ccccc67)cc5)n4)c23)cc1 ; c1ccc(-c2ccc3c(c2)oc2cccc(-c4nc(-c5ccccc5)nc(-c5ccc(-c6ccc7c(ccc8ccccc87)c6)cc5)n4)c23)cc1]

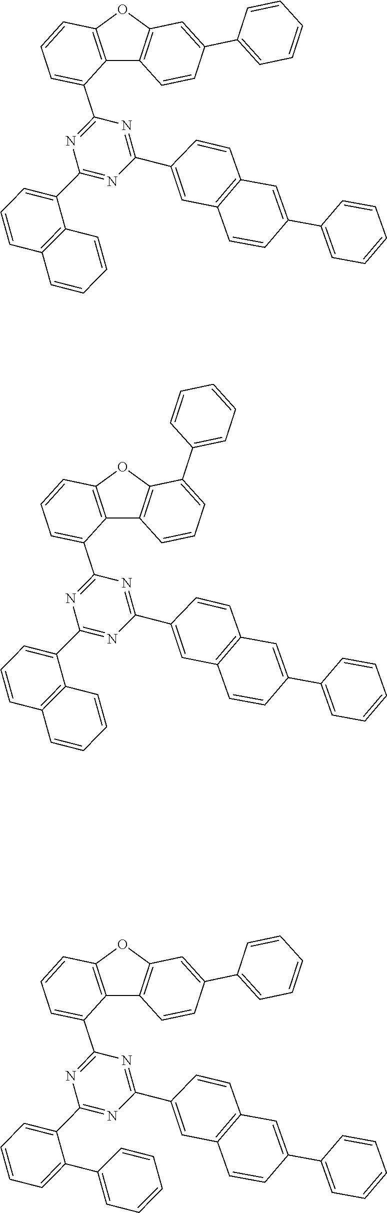 [SMILES: c1ccc(-c2ccc3cc(-c4nc(-c5cccc6ccccc56)nc(-c5cccc6oc7cc(-c8ccccc8)ccc7c56)n4)ccc3c2)cc1 ; c1ccc(-c2ccc3cc(-c4nc(-c5cccc6ccccc56)nc(-c5cccc6oc7c(-c8ccccc8)cccc7c56)n4)ccc3c2)cc1 ; c1ccc(-c2ccc3cc(-c4nc(-c5ccccc5-c5ccccc5)nc(-c5cccc6oc7cc(-c8ccccc8)ccc7c56)n4)ccc3c2)cc1]

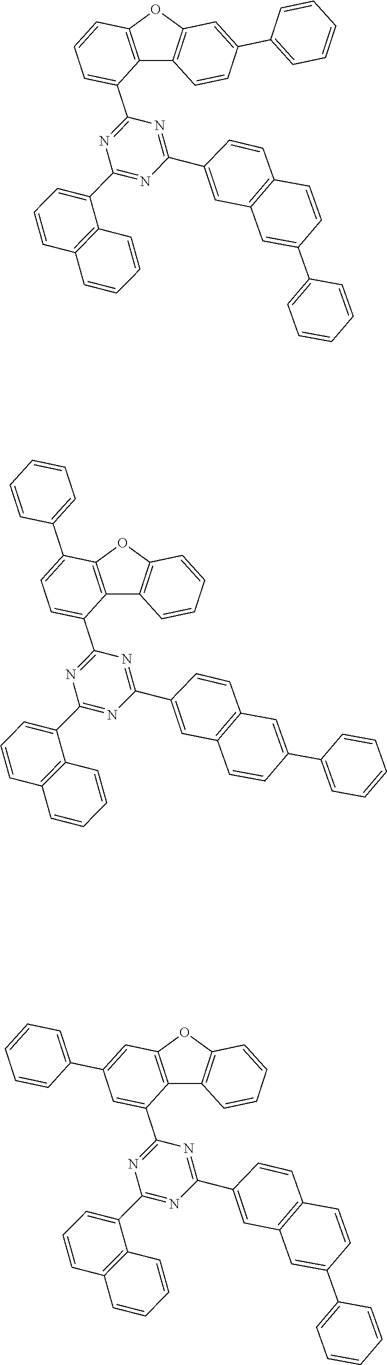 [SMILES: c1ccc(-c2ccc3ccc(-c4nc(-c5cccc6ccccc56)nc(-c5cccc6oc7cc(-c8ccccc8)ccc7c56)n4)cc3c2)cc1 ; c1ccc(-c2ccc3cc(-c4nc(-c5cccc6ccccc56)nc(-c5ccc(-c6ccccc6)c6oc7ccccc7c56)n4)ccc3c2)cc1 ; c1ccc(-c2ccc3ccc(-c4nc(-c5cccc6ccccc56)nc(-c5cc(-c6ccccc6)cc6oc7ccccc7c56)n4)cc3c2)cc1]

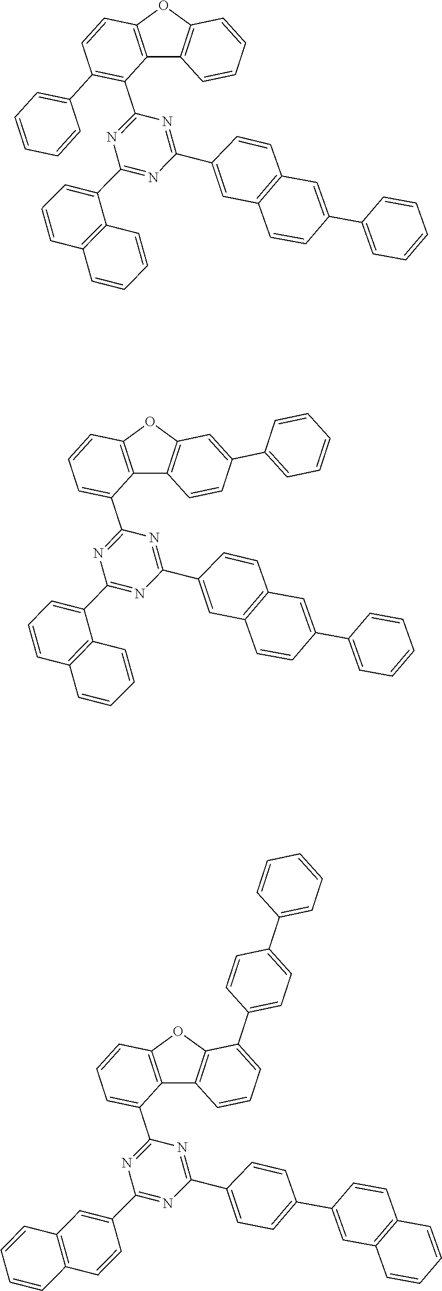 [SMILES: c1ccc(-c2ccc3cc(-c4nc(-c5cccc6ccccc56)nc(-c5c(-c6ccccc6)ccc6oc7ccccc7c56)n4)ccc3c2)cc1 ; c1ccc(-c2ccc3cc(-c4nc(-c5cccc6ccccc56)nc(-c5cccc6oc7cc(-c8ccccc8)ccc7c56)n4)ccc3c2)cc1 ; c1ccc(-c2ccc(-c3cccc4c3oc3cccc(-c5nc(-c6ccc(-c7ccc8ccccc8c7)cc6)nc(-c6ccc7ccccc7c6)n5)c34)cc2)cc1]

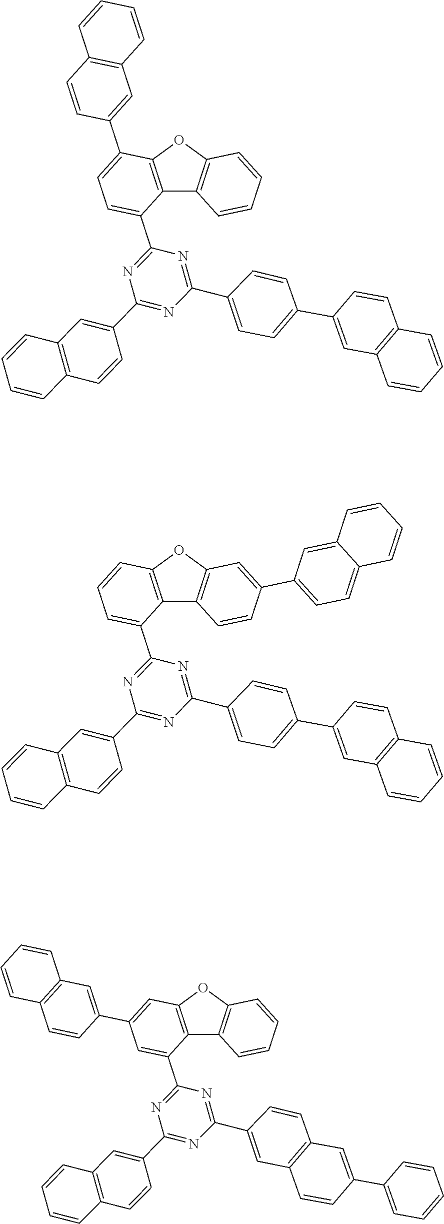 [SMILES: c1ccc2cc(-c3ccc(-c4nc(-c5ccc6ccccc6c5)nc(-c5ccc(-c6ccc7ccccc7c6)c6oc7ccccc7c56)n4)cc3)ccc2c1 ; c1ccc2cc(-c3ccc(-c4nc(-c5ccc6ccccc6c5)nc(-c5cccc6oc7cc(-c8ccc9ccccc9c8)ccc7c56)n4)cc3)ccc2c1 ; c1ccc(-c2ccc3cc(-c4nc(-c5ccc6ccccc6c5)nc(-c5cc(-c6ccc7ccccc7c6)cc6oc7ccccc7c56)n4)ccc3c2)cc1]

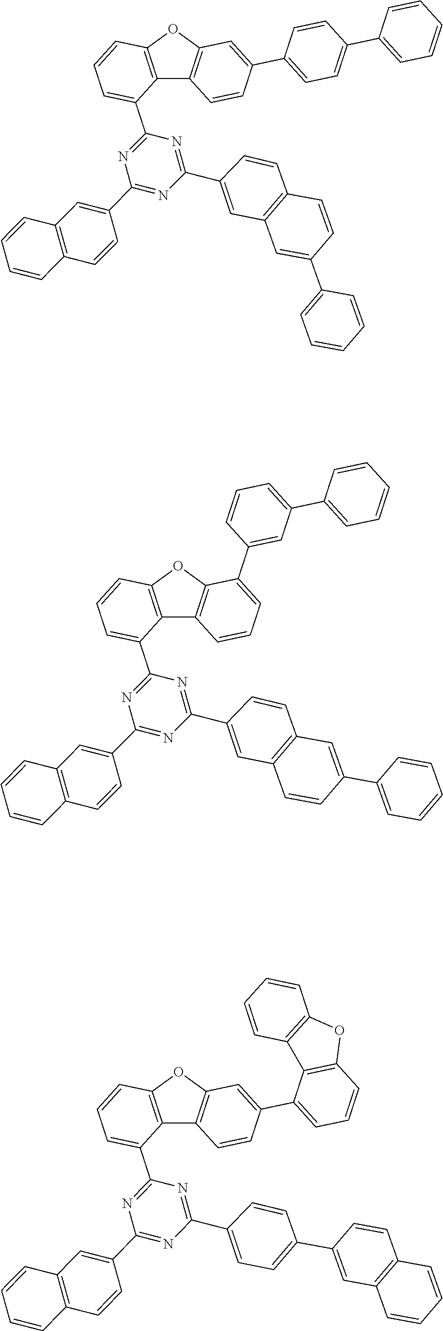 [SMILES: c1ccc(-c2ccc(-c3ccc4c(c3)oc3cccc(-c5nc(-c6ccc7ccccc7c6)nc(-c6ccc7ccc(-c8ccccc8)cc7c6)n5)c34)cc2)cc1 ; c1ccc(-c2cccc(-c3cccc4c3oc3cccc(-c5nc(-c6ccc7ccccc7c6)nc(-c6ccc7cc(-c8ccccc8)ccc7c6)n5)c34)c2)cc1 ; c1ccc2cc(-c3ccc(-c4nc(-c5ccc6ccccc6c5)nc(-c5cccc6oc7cc(-c8cccc9oc%10ccccc%10c89)ccc7c56)n4)cc3)ccc2c1]

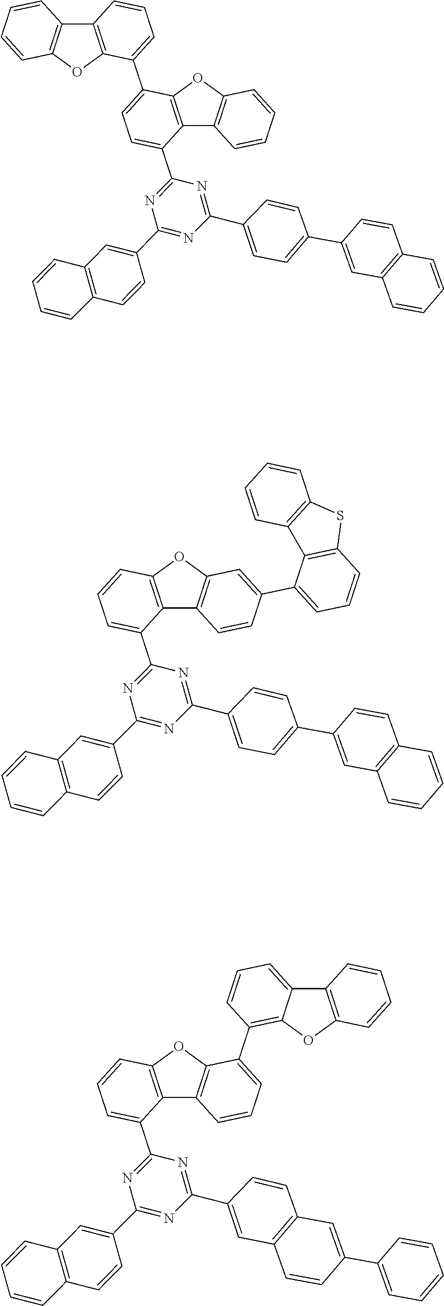 [SMILES: c1ccc2cc(-c3ccc(-c4nc(-c5ccc6ccccc6c5)nc(-c5ccc(-c6cccc7c6oc6ccccc67)c6oc7ccccc7c56)n4)cc3)ccc2c1 ; c1ccc2cc(-c3ccc(-c4nc(-c5ccc6ccccc6c5)nc(-c5cccc6oc7cc(-c8cccc9sc%10ccccc%10c89)ccc7c56)n4)cc3)ccc2c1 ; c1ccc(-c2ccc3cc(-c4nc(-c5ccc6ccccc6c5)nc(-c5cccc6oc7c(-c8cccc9c8oc8ccccc89)cccc7c56)n4)ccc3c2)cc1]

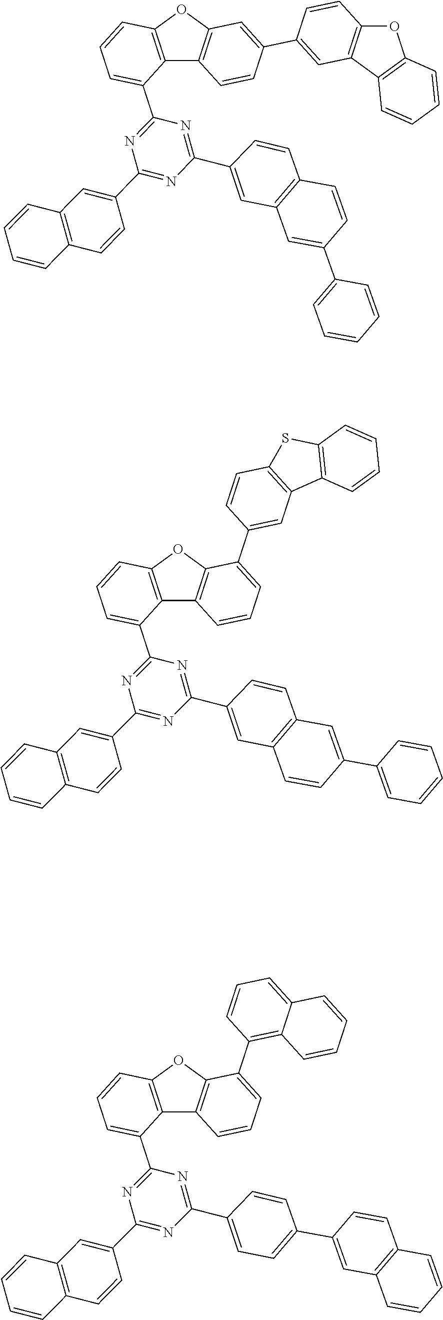 [SMILES: c1ccc(-c2ccc3ccc(-c4nc(-c5ccc6ccccc6c5)nc(-c5cccc6oc7cc(-c8ccc9oc%10ccccc%10c9c8)ccc7c56)n4)cc3c2)cc1 ; c1ccc(-c2ccc3cc(-c4nc(-c5ccc6ccccc6c5)nc(-c5cccc6oc7c(-c8ccc9sc%10ccccc%10c9c8)cccc7c56)n4)ccc3c2)cc1 ; c1ccc2cc(-c3ccc(-c4nc(-c5ccc6ccccc6c5)nc(-c5cccc6oc7c(-c8cccc9ccccc89)cccc7c56)n4)cc3)ccc2c1]

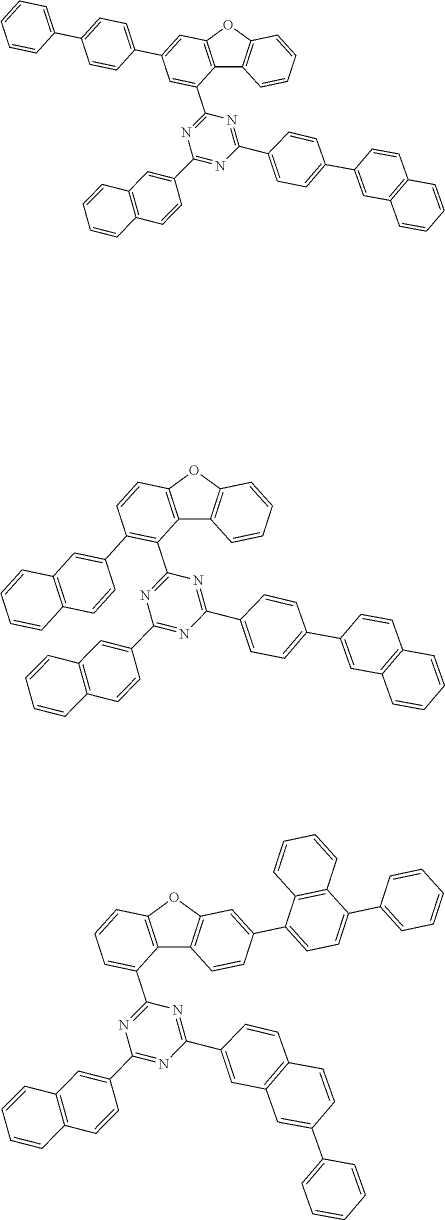 [SMILES: c1ccc(-c2ccc(-c3cc(-c4nc(-c5ccc(-c6ccc7ccccc7c6)cc5)nc(-c5ccc6ccccc6c5)n4)c4c(c3)oc3ccccc34)cc2)cc1 ; c1ccc2cc(-c3ccc(-c4nc(-c5ccc6ccccc6c5)nc(-c5c(-c6ccc7ccccc7c6)ccc6oc7ccccc7c56)n4)cc3)ccc2c1 ; c1ccc(-c2ccc3ccc(-c4nc(-c5ccc6ccccc6c5)nc(-c5cccc6oc7cc(-c8ccc(-c9ccccc9)c9ccccc89)ccc7c56)n4)cc3c2)cc1]

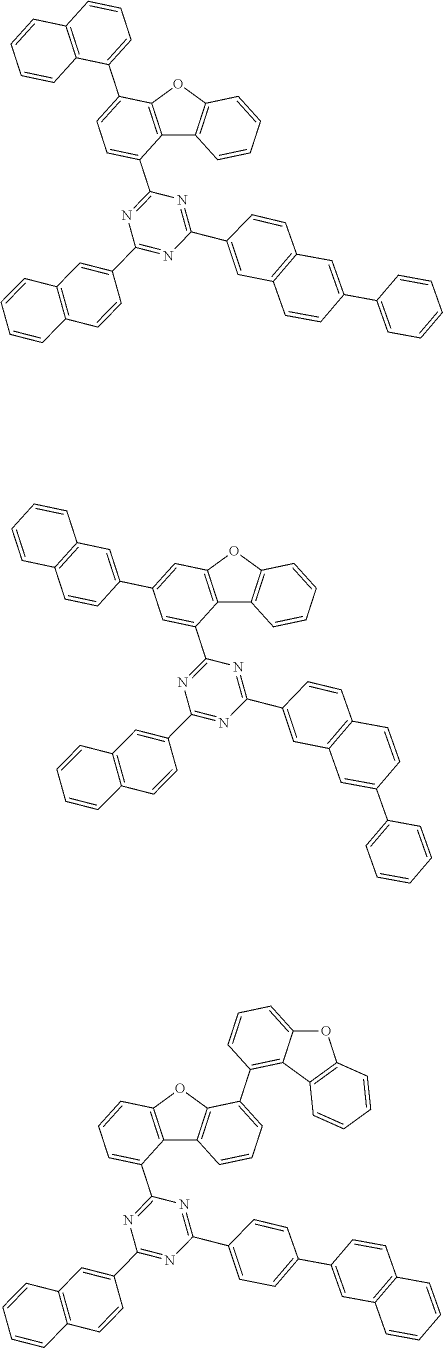 [SMILES: c1ccc(-c2ccc3cc(-c4nc(-c5ccc6ccccc6c5)nc(-c5ccc(-c6cccc7ccccc67)c6oc7ccccc7c56)n4)ccc3c2)cc1 ; c1ccc(-c2ccc3ccc(-c4nc(-c5ccc6ccccc6c5)nc(-c5cc(-c6ccc7ccccc7c6)cc6oc7ccccc7c56)n4)cc3c2)cc1 ; c1ccc2cc(-c3ccc(-c4nc(-c5ccc6ccccc6c5)nc(-c5cccc6oc7c(-c8cccc9oc%10ccccc%10c89)cccc7c56)n4)cc3)ccc2c1]

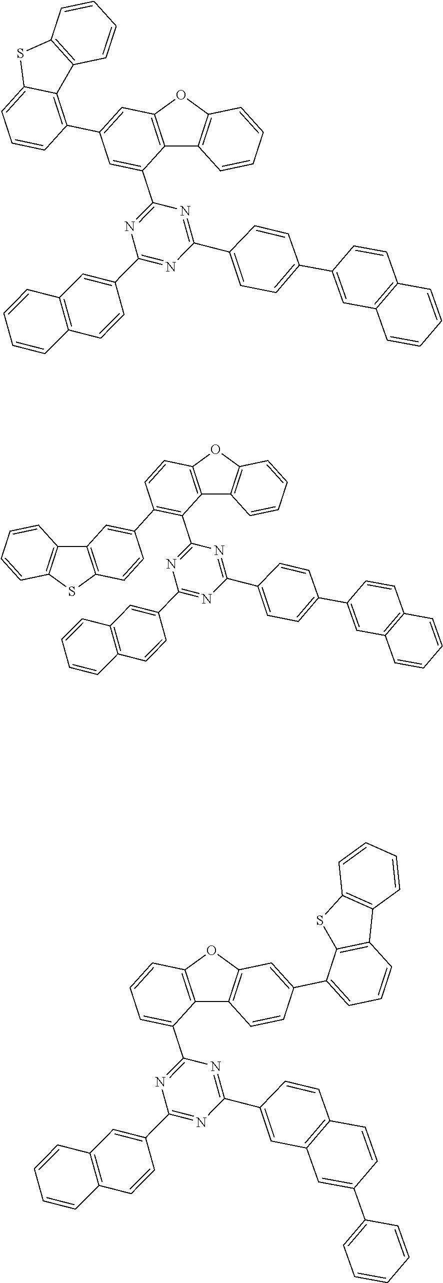 [SMILES: c1ccc2cc(-c3ccc(-c4nc(-c5ccc6ccccc6c5)nc(-c5cc(-c6cccc7sc8ccccc8c67)cc6oc7ccccc7c56)n4)cc3)ccc2c1 ; c1ccc2cc(-c3ccc(-c4nc(-c5ccc6ccccc6c5)nc(-c5c(-c6ccc7sc8ccccc8c7c6)ccc6oc7ccccc7c56)n4)cc3)ccc2c1 ; c1ccc(-c2ccc3ccc(-c4nc(-c5ccc6ccccc6c5)nc(-c5cccc6oc7cc(-c8cccc9c8sc8ccccc89)ccc7c56)n4)cc3c2)cc1]

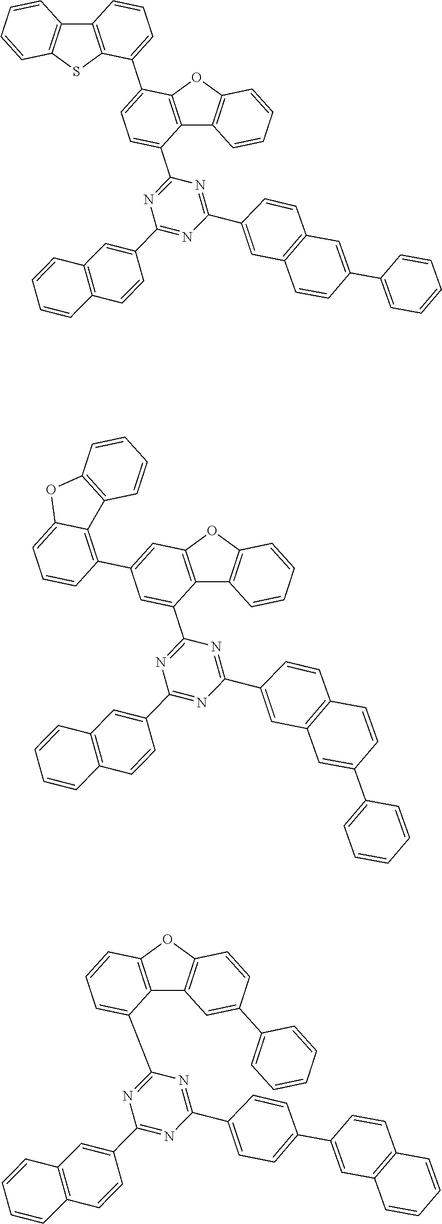 [SMILES: c1ccc(-c2ccc3cc(-c4nc(-c5ccc6ccccc6c5)nc(-c5ccc(-c6cccc7c6sc6ccccc67)c6oc7ccccc7c56)n4)ccc3c2)cc1 ; c1ccc(-c2ccc3ccc(-c4nc(-c5ccc6ccccc6c5)nc(-c5cc(-c6cccc7oc8ccccc8c67)cc6oc7ccccc7c56)n4)cc3c2)cc1 ; c1ccc(-c2ccc3oc4cccc(-c5nc(-c6ccc(-c7ccc8ccccc8c7)cc6)nc(-c6ccc7ccccc7c6)n5)c4c3c2)cc1]

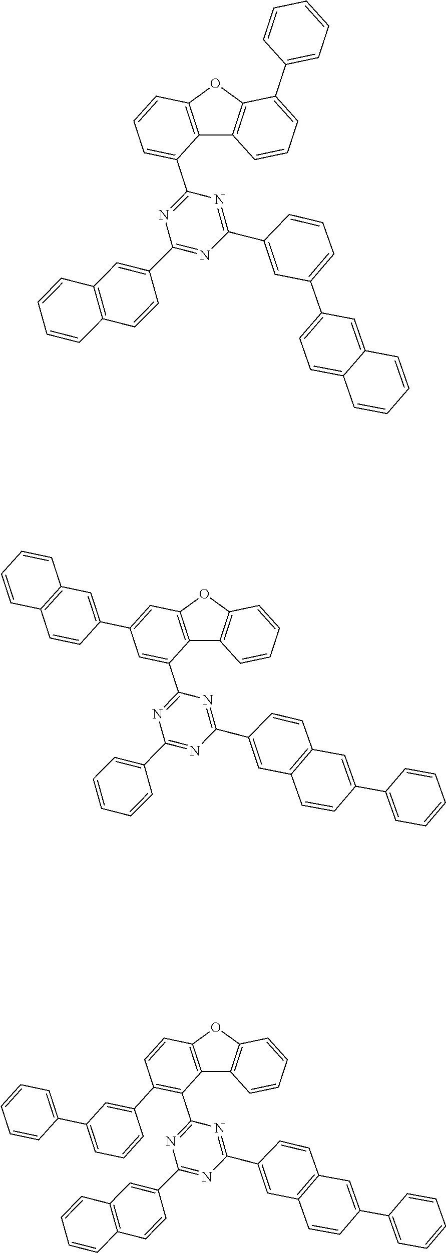 [SMILES: c1ccc(-c2cccc3c2oc2cccc(-c4nc(-c5cccc(-c6ccc7ccccc7c6)c5)nc(-c5ccc6ccccc6c5)n4)c23)cc1 ; c1ccc(-c2ccc3cc(-c4nc(-c5ccccc5)nc(-c5cc(-c6ccc7ccccc7c6)cc6oc7ccccc7c56)n4)ccc3c2)cc1 ; c1ccc(-c2cccc(-c3ccc4oc5ccccc5c4c3-c3nc(-c4ccc5ccccc5c4)nc(-c4ccc5cc(-c6ccccc6)ccc5c4)n3)c2)cc1]

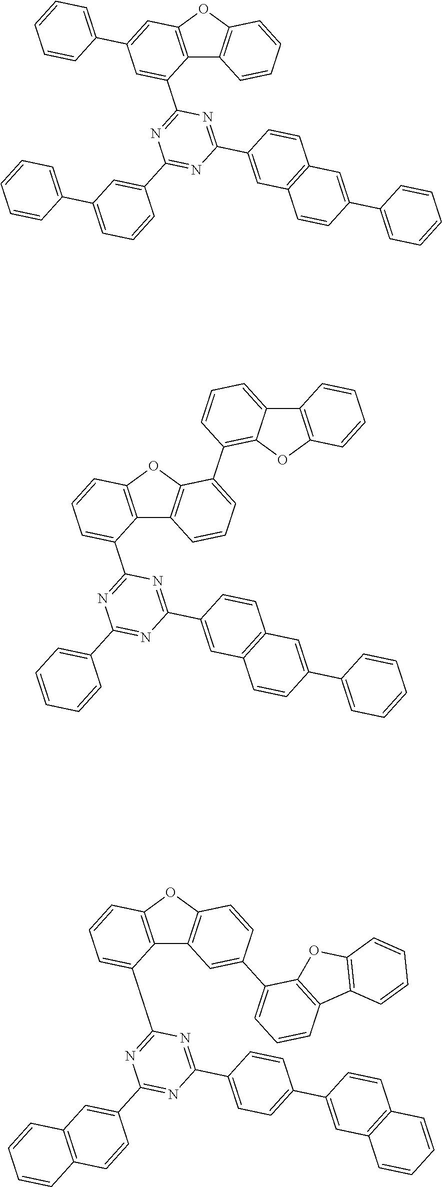 [SMILES: c1ccc(-c2cccc(-c3nc(-c4ccc5cc(-c6ccccc6)ccc5c4)nc(-c4cc(-c5ccccc5)cc5oc6ccccc6c45)n3)c2)cc1 ; c1ccc(-c2ccc3cc(-c4nc(-c5ccccc5)nc(-c5cccc6oc7c(-c8cccc9c8oc8ccccc89)cccc7c56)n4)ccc3c2)cc1 ; c1ccc2cc(-c3ccc(-c4nc(-c5ccc6ccccc6c5)nc(-c5cccc6oc7ccc(-c8cccc9c8oc8ccccc89)cc7c56)n4)cc3)ccc2c1]

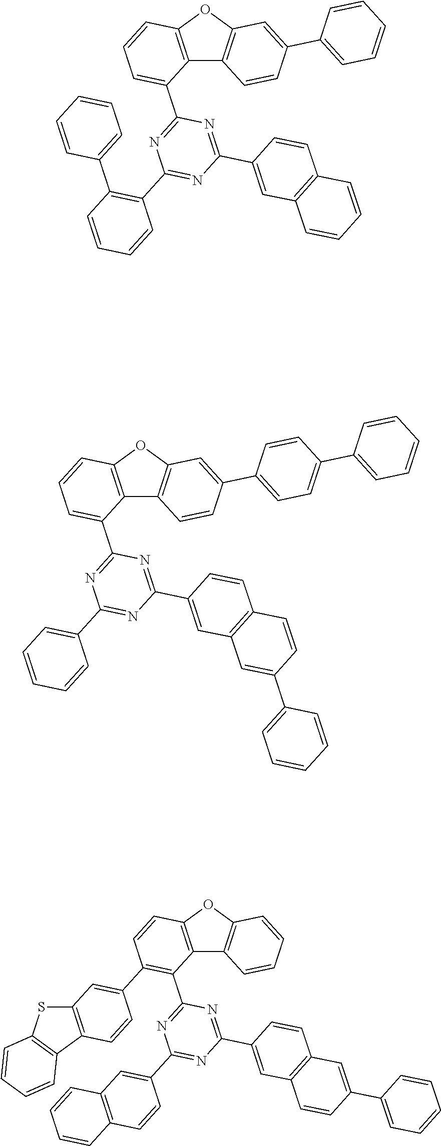 [SMILES: c1ccc(-c2ccc3c(c2)oc2cccc(-c4nc(-c5ccc6ccccc6c5)nc(-c5ccccc5-c5ccccc5)n4)c23)cc1 ; c1ccc(-c2ccc(-c3ccc4c(c3)oc3cccc(-c5nc(-c6ccccc6)nc(-c6ccc7ccc(-c8ccccc8)cc7c6)n5)c34)cc2)cc1 ; c1ccc(-c2ccc3cc(-c4nc(-c5ccc6ccccc6c5)nc(-c5c(-c6ccc7c(c6)sc6ccccc67)ccc6oc7ccccc7c56)n4)ccc3c2)cc1]

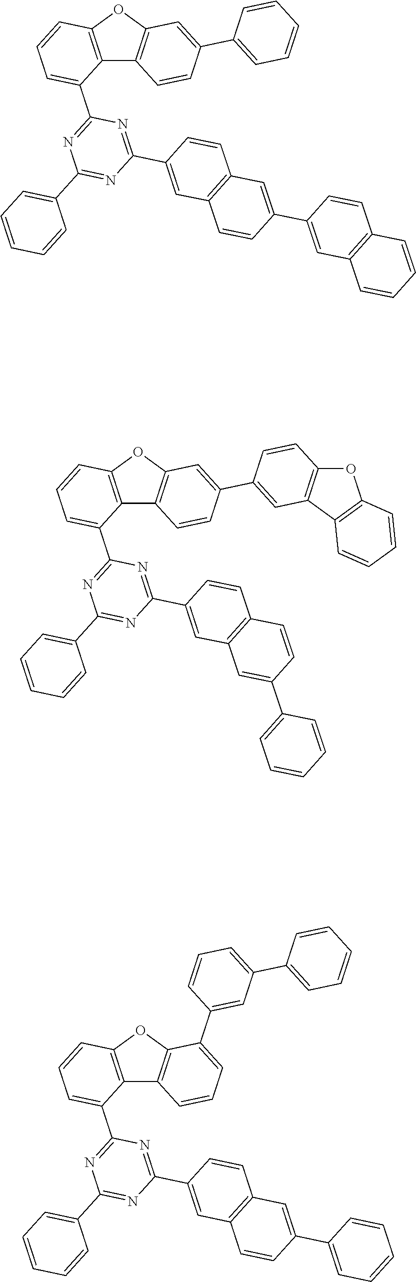 [SMILES: c1ccc(-c2ccc3c(c2)oc2cccc(-c4nc(-c5ccccc5)nc(-c5ccc6cc(-c7ccc8ccccc8c7)ccc6c5)n4)c23)cc1 ; c1ccc(-c2ccc3ccc(-c4nc(-c5ccccc5)nc(-c5cccc6oc7cc(-c8ccc9oc%10ccccc%10c9c8)ccc7c56)n4)cc3c2)cc1 ; c1ccc(-c2cccc(-c3cccc4c3oc3cccc(-c5nc(-c6ccccc6)nc(-c6ccc7cc(-c8ccccc8)ccc7c6)n5)c34)c2)cc1]

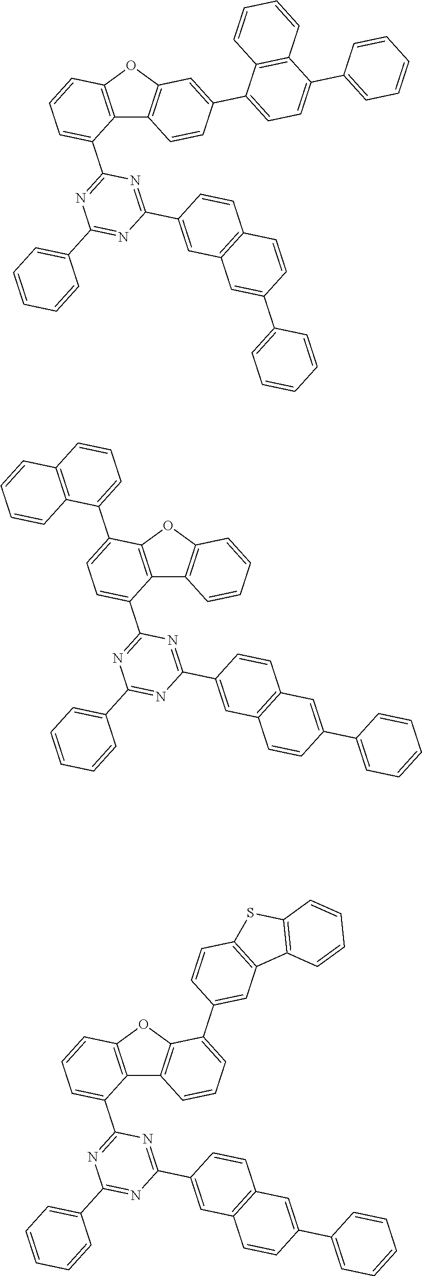 [SMILES: c1ccc(-c2ccc3ccc(-c4nc(-c5ccccc5)nc(-c5cccc6oc7cc(-c8ccc(-c9ccccc9)c9ccccc89)ccc7c56)n4)cc3c2)cc1 ; c1ccc(-c2ccc3cc(-c4nc(-c5ccccc5)nc(-c5ccc(-c6cccc7ccccc67)c6oc7ccccc7c56)n4)ccc3c2)cc1 ; c1ccc(-c2ccc3cc(-c4nc(-c5ccccc5)nc(-c5cccc6oc7c(-c8ccc9sc%10ccccc%10c9c8)cccc7c56)n4)ccc3c2)cc1]

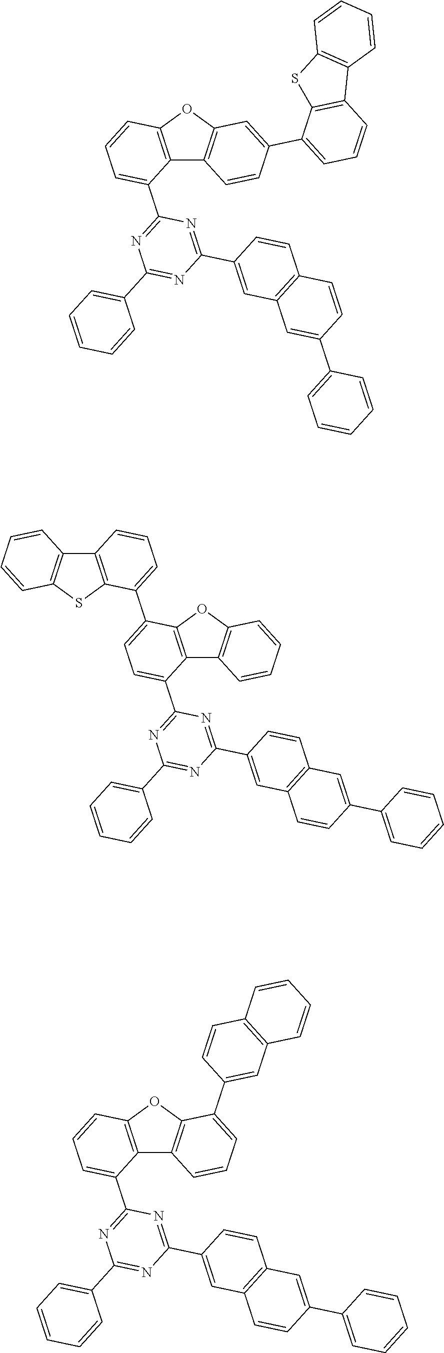 [SMILES: c1ccc(-c2ccc3ccc(-c4nc(-c5ccccc5)nc(-c5cccc6oc7cc(-c8cccc9c8sc8ccccc89)ccc7c56)n4)cc3c2)cc1 ; c1ccc(-c2ccc3cc(-c4nc(-c5ccccc5)nc(-c5ccc(-c6cccc7c6sc6ccccc67)c6oc7ccccc7c56)n4)ccc3c2)cc1 ; c1ccc(-c2ccc3cc(-c4nc(-c5ccccc5)nc(-c5cccc6oc7c(-c8ccc9ccccc9c8)cccc7c56)n4)ccc3c2)cc1]

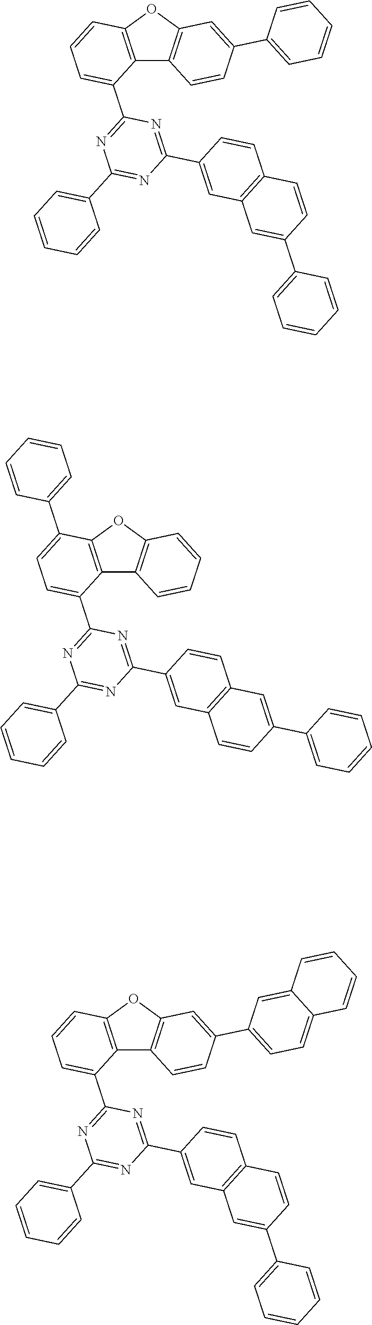 [SMILES: c1ccc(-c2ccc3ccc(-c4nc(-c5ccccc5)nc(-c5cccc6oc7cc(-c8ccccc8)ccc7c56)n4)cc3c2)cc1 ; c1ccc(-c2ccc3cc(-c4nc(-c5ccccc5)nc(-c5ccc(-c6ccccc6)c6oc7ccccc7c56)n4)ccc3c2)cc1 ; c1ccc(-c2ccc3ccc(-c4nc(-c5ccccc5)nc(-c5cccc6oc7cc(-c8ccc9ccccc9c8)ccc7c56)n4)cc3c2)cc1]

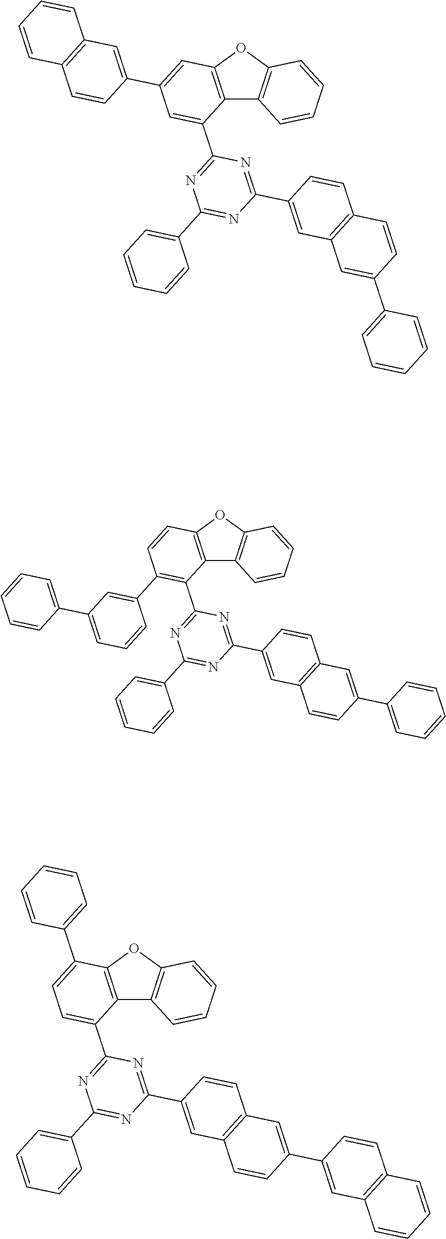 [SMILES: c1ccc(-c2ccc3ccc(-c4nc(-c5ccccc5)nc(-c5cc(-c6ccc7ccccc7c6)cc6oc7ccccc7c56)n4)cc3c2)cc1 ; c1ccc(-c2cccc(-c3ccc4oc5ccccc5c4c3-c3nc(-c4ccccc4)nc(-c4ccc5cc(-c6ccccc6)ccc5c4)n3)c2)cc1 ; c1ccc(-c2nc(-c3ccc4cc(-c5ccc6ccccc6c5)ccc4c3)nc(-c3ccc(-c4ccccc4)c4oc5ccccc5c34)n2)cc1]

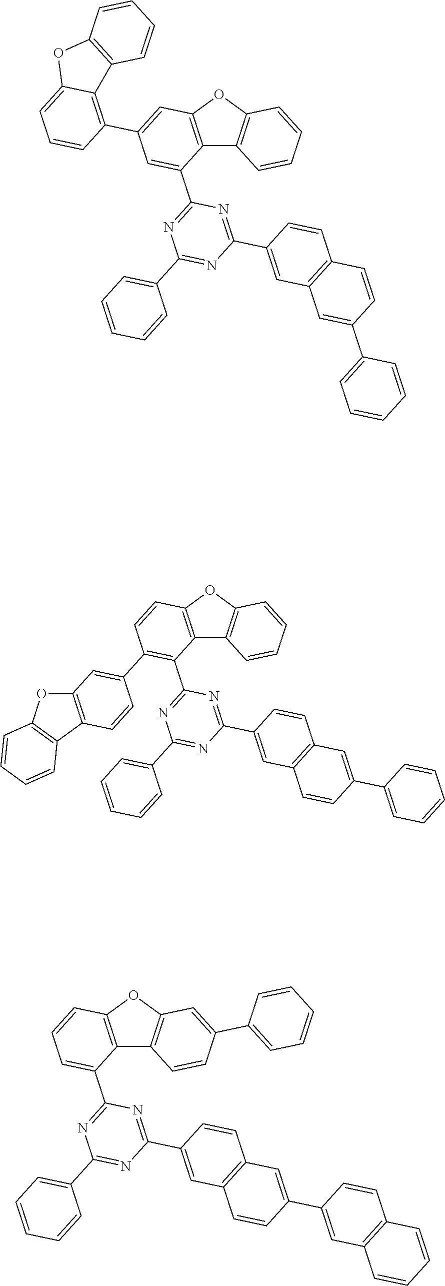 [SMILES: c1ccc(-c2ccc3ccc(-c4nc(-c5ccccc5)nc(-c5cc(-c6cccc7oc8ccccc8c67)cc6oc7ccccc7c56)n4)cc3c2)cc1 ; c1ccc(-c2ccc3cc(-c4nc(-c5ccccc5)nc(-c5c(-c6ccc7c(c6)oc6ccccc67)ccc6oc7ccccc7c56)n4)ccc3c2)cc1 ; c1ccc(-c2ccc3c(c2)oc2cccc(-c4nc(-c5ccccc5)nc(-c5ccc6cc(-c7ccc8ccccc8c7)ccc6c5)n4)c23)cc1]

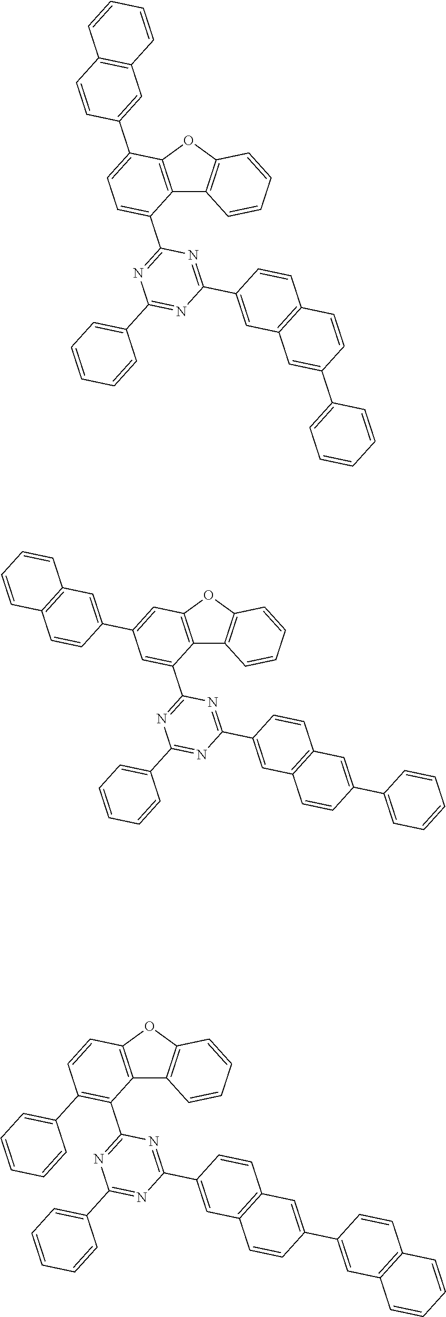 [SMILES: c1ccc(-c2ccc3ccc(-c4nc(-c5ccccc5)nc(-c5ccc(-c6ccc7ccccc7c6)c6oc7ccccc7c56)n4)cc3c2)cc1 ; c1ccc(-c2ccc3cc(-c4nc(-c5ccccc5)nc(-c5cc(-c6ccc7ccccc7c6)cc6oc7ccccc7c56)n4)ccc3c2)cc1 ; c1ccc(-c2nc(-c3ccc4cc(-c5ccc6ccccc6c5)ccc4c3)nc(-c3c(-c4ccccc4)ccc4oc5ccccc5c34)n2)cc1]

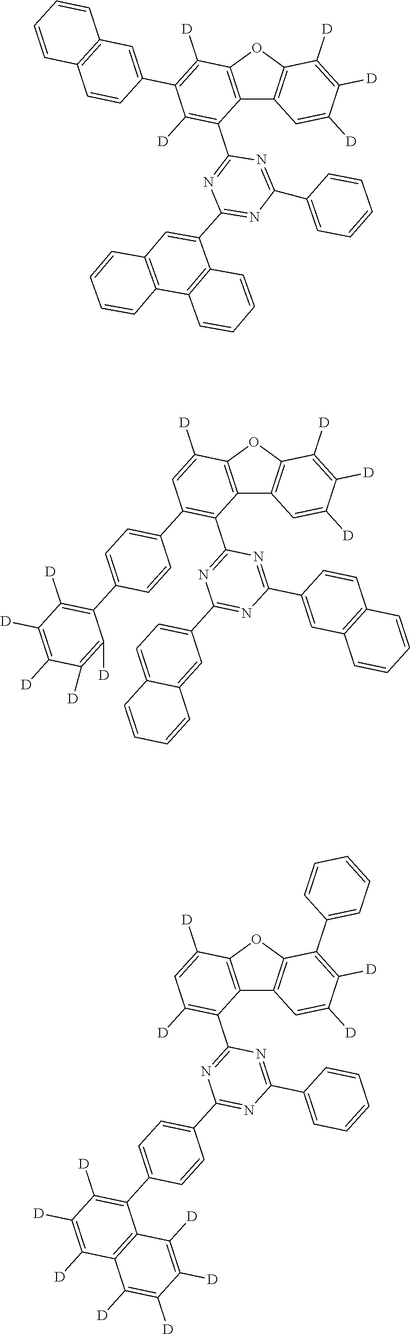 [SMILES: [2H]c1cc2c(oc3c([2H])c(-c4ccc5ccccc5c4)c([2H])c(-c4nc(-c5ccccc5)nc(-c5cc6ccccc6c6ccccc56)n4)c32)c([2H])c1[2H] ; [2H]c1cc2c(oc3c([2H])cc(-c4ccc(-c5c([2H])c([2H])c([2H])c([2H])c5[2H])cc4)c(-c4nc(-c5ccc6ccccc6c5)nc(-c5ccc6ccccc6c5)n4)c32)c([2H])c1[2H] ; [2H]c1cc2c(oc3c([2H])cc([2H])c(-c4nc(-c5ccccc5)nc(-c5ccc(-c6c([2H])c([2H])c([2H])c7c([2H])c([2H])c([2H])c([2H])c67)cc5)n4)c32)c(-c2ccccc2)c1[2H]]

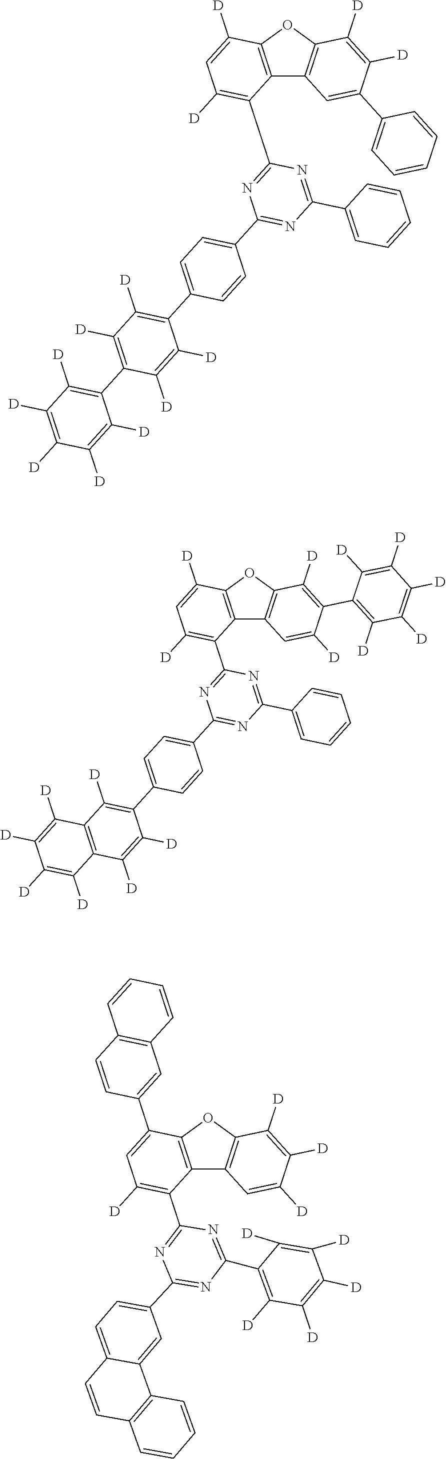 [SMILES: [2H]c1cc([2H])c2oc3c([2H])c([2H])c(-c4ccccc4)cc3c2c1-c1nc(-c2ccccc2)nc(-c2ccc(-c3c([2H])c([2H])c(-c4c([2H])c([2H])c([2H])c([2H])c4[2H])c([2H])c3[2H])cc2)n1 ; [2H]c1cc2c(oc3c([2H])cc([2H])c(-c4nc(-c5ccccc5)nc(-c5ccc(-c6c([2H])c([2H])c7c([2H])c([2H])c([2H])c([2H])c7c6[2H])cc5)n4)c32)c([2H])c1-c1c([2H])c([2H])c([2H])c([2H])c1[2H] ; [2H]c1cc2c(oc3c(-c4ccc5ccccc5c4)cc([2H])c(-c4nc(-c5ccc6ccc7ccccc7c6c5)nc(-c5c([2H])c([2H])c([2H])c([2H])c5[2H])n4)c32)c([2H])c1[2H]]

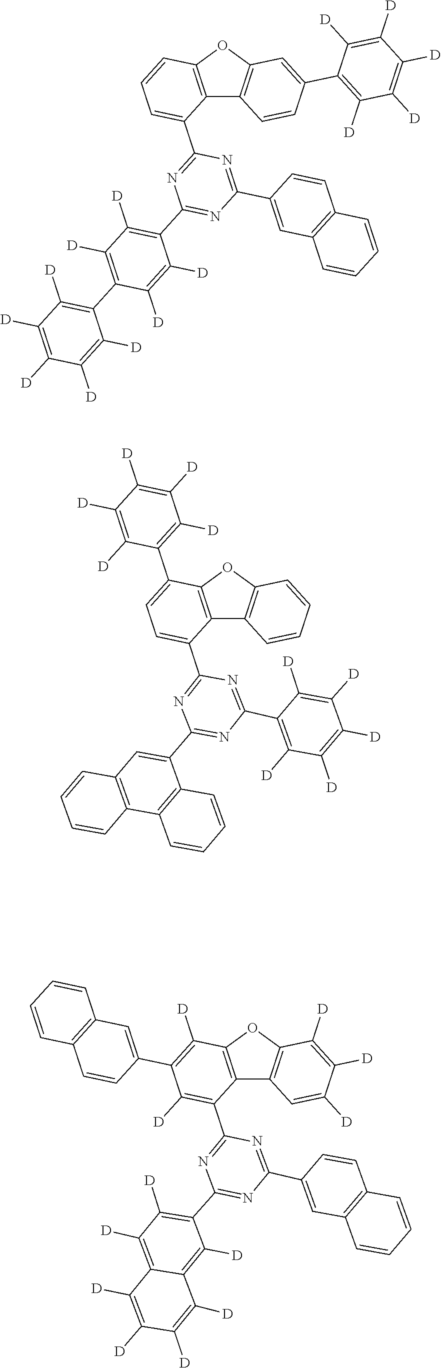 [SMILES: [2H]c1c([2H])c([2H])c(-c2ccc3c(c2)oc2cccc(-c4nc(-c5ccc6ccccc6c5)nc(-c5c([2H])c([2H])c(-c6c([2H])c([2H])c([2H])c([2H])c6[2H])c([2H])c5[2H])n4)c23)c([2H])c1[2H] ; [2H]c1c([2H])c([2H])c(-c2nc(-c3cc4ccccc4c4ccccc34)nc(-c3ccc(-c4c([2H])c([2H])c([2H])c([2H])c4[2H])c4oc5ccccc5c34)n2)c([2H])c1[2H] ; [2H]c1cc2c(oc3c([2H])c(-c4ccc5ccccc5c4)c([2H])c(-c4nc(-c5ccc6ccccc6c5)nc(-c5c([2H])c([2H])c6c([2H])c([2H])c([2H])c([2H])c6c5[2H])n4)c32)c([2H])c1[2H]]

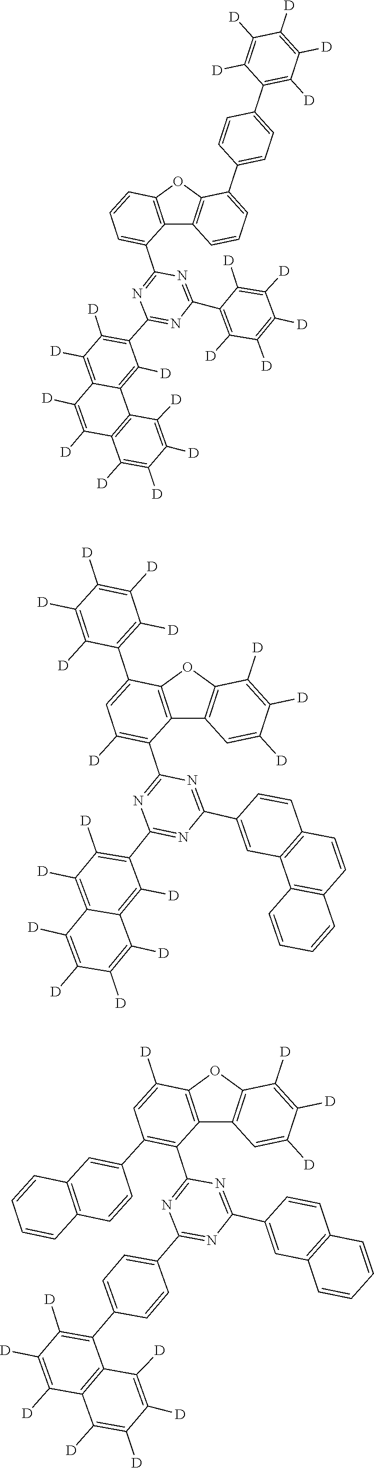

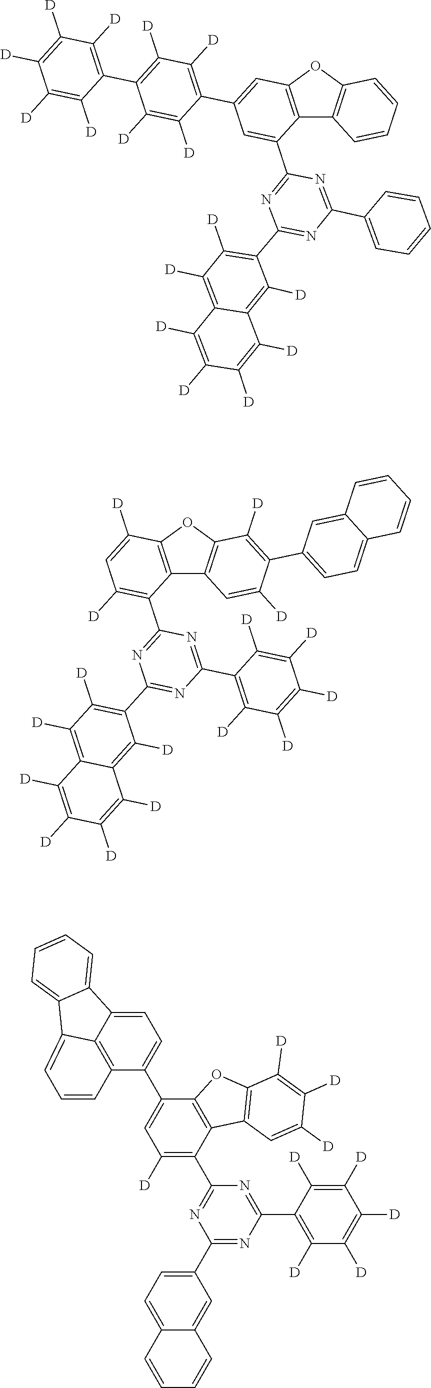 [SMILES: [2H]c1c([2H])c([2H])c(-c2c([2H])c([2H])c(-c3cc(-c4nc(-c5ccccc5)nc(-c5c([2H])c([2H])c6c([2H])c([2H])c([2H])c([2H])c6c5[2H])n4)c4c(c3)oc3ccccc34)c([2H])c2[2H])c([2H])c1[2H] ; [2H]c1cc2c(oc3c([2H])cc([2H])c(-c4nc(-c5c([2H])c([2H])c([2H])c([2H])c5[2H])nc(-c5c([2H])c([2H])c6c([2H])c([2H])c([2H])c([2H])c6c5[2H])n4)c32)c([2H])c1-c1ccc2ccccc2c1 ; [2H]c1cc2c(oc3c(-c4ccc5c6c(cccc46)-c4ccccc4-5)cc([2H])c(-c4nc(-c5ccc6ccccc6c5)nc(-c5c([2H])c([2H])c([2H])c([2H])c5[2H])n4)c32)c([2H])c1[2H]]

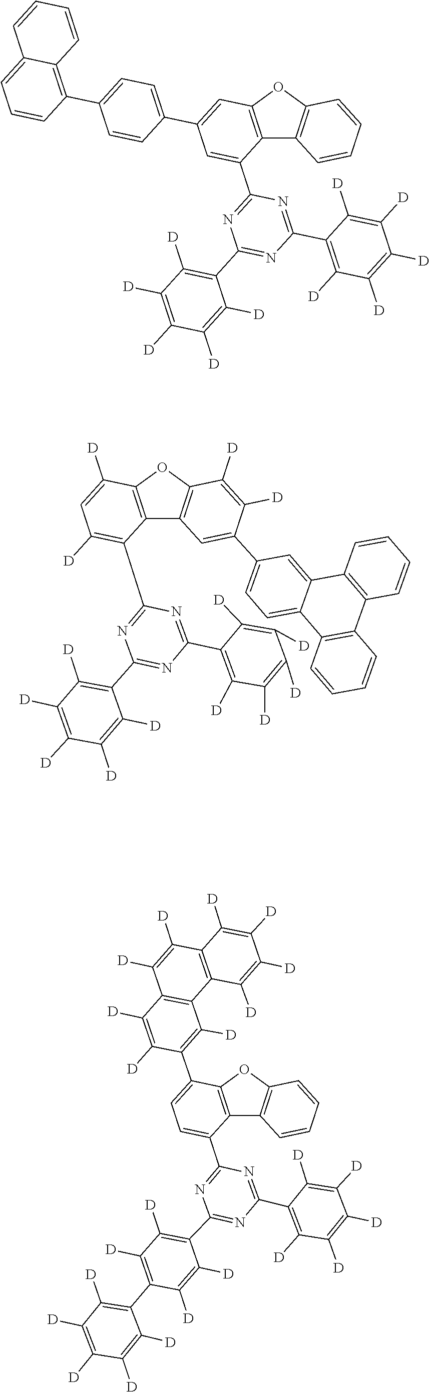

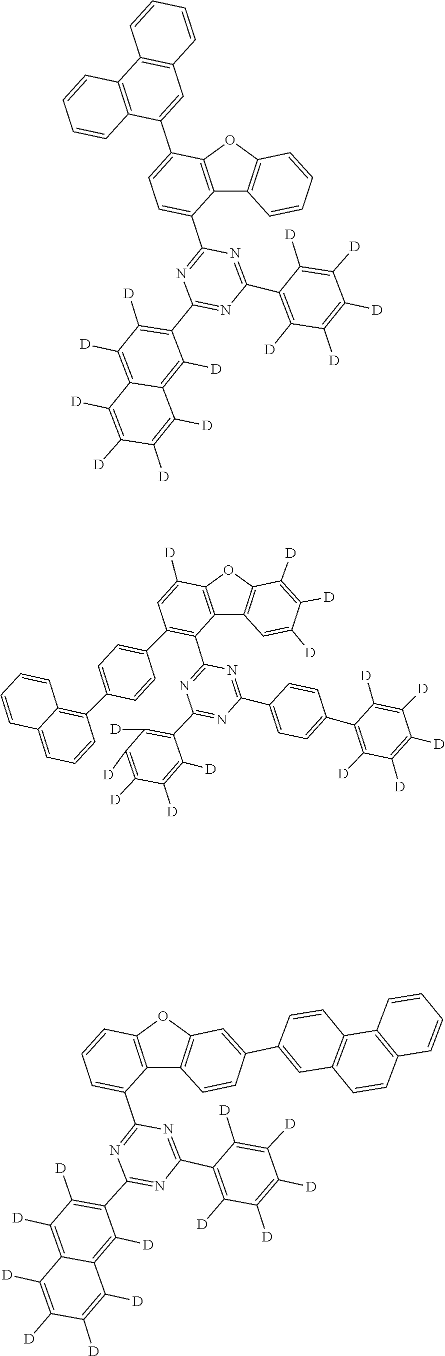 [SMILES: [2H]c1c([2H])c([2H])c(-c2nc(-c3c([2H])c([2H])c4c([2H])c([2H])c([2H])c([2H])c4c3[2H])nc(-c3ccc(-c4cc5ccccc5c5ccccc45)c4oc5ccccc5c34)n2)c([2H])c1[2H] ; [2H]c1cc2c(oc3c([2H])cc(-c4ccc(-c5cccc6ccccc56)cc4)c(-c4nc(-c5ccc(-c6c([2H])c([2H])c([2H])c([2H])c6[2H])cc5)nc(-c5c([2H])c([2H])c([2H])c([2H])c5[2H])n4)c32)c([2H])c1[2H] ; [2H]c1c([2H])c([2H])c(-c2nc(-c3c([2H])c([2H])c4c([2H])c([2H])c([2H])c([2H])c4c3[2H])nc(-c3cccc4oc5cc(-c6ccc7c(ccc8ccccc87)c6)ccc5c34)n2)c([2H])c1[2H]]

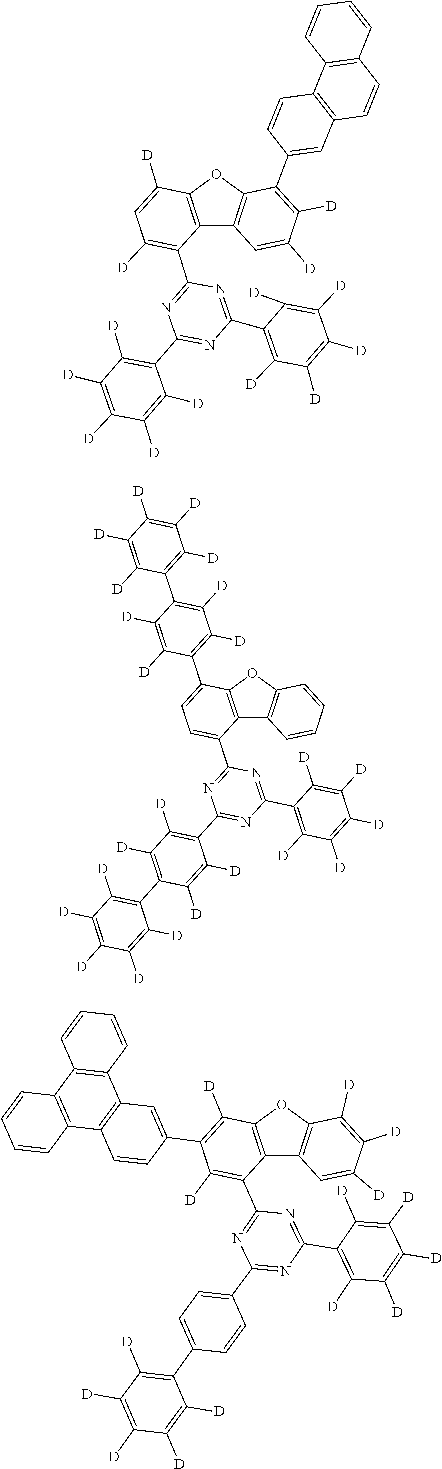

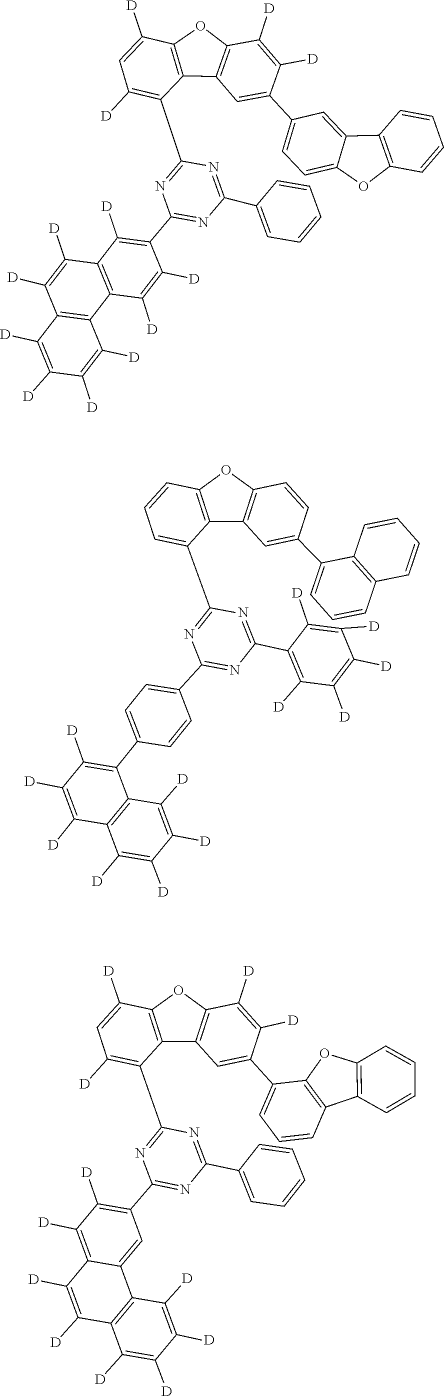 [SMILES: [2H]c1cc([2H])c2oc3c([2H])c([2H])c(-c4ccc5oc6ccccc6c5c4)cc3c2c1-c1nc(-c2ccccc2)nc(-c2c([2H])c([2H])c3c(c2[2H])c([2H])c([2H])c2c([2H])c([2H])c([2H])c([2H])c23)n1 ; [2H]c1c([2H])c([2H])c(-c2nc(-c3ccc(-c4c([2H])c([2H])c([2H])c5c([2H])c([2H])c([2H])c([2H])c45)cc3)nc(-c3cccc4oc5ccc(-c6cccc7ccccc67)cc5c34)n2)c([2H])c1[2H] ; [2H]c1cc([2H])c2oc3c([2H])c([2H])c(-c4cccc5c4oc4ccccc45)cc3c2c1-c1nc(-c2ccccc2)nc(-c2cc3c(c([2H])c2[2H])c([2H])c([2H])c2c([2H])c([2H])c([2H])c([2H])c23)n1]

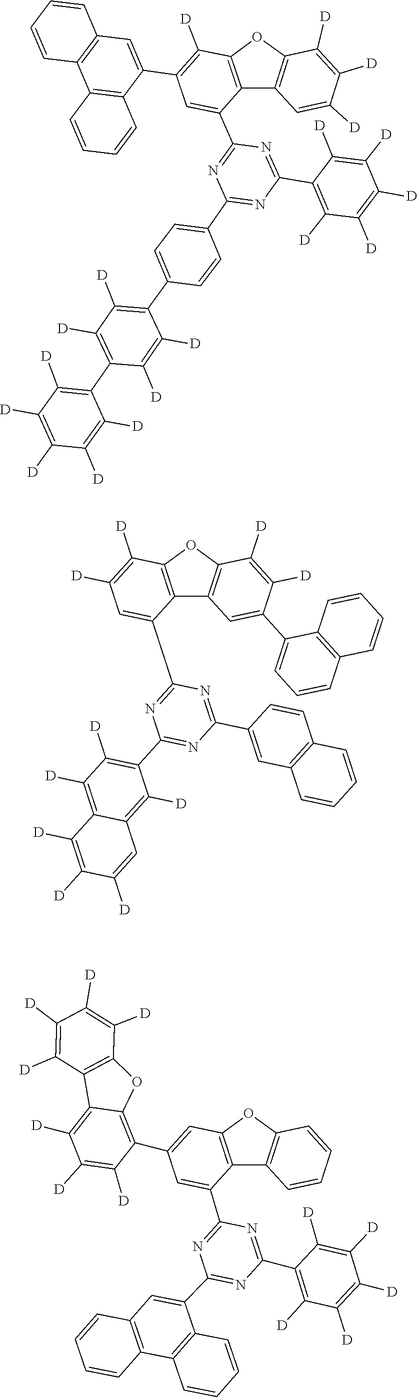 [SMILES: [2H]c1cc2c(oc3c([2H])c(-c4cc5ccccc5c5ccccc45)cc(-c4nc(-c5ccc(-c6c([2H])c([2H])c(-c7c([2H])c([2H])c([2H])c([2H])c7[2H])c([2H])c6[2H])cc5)nc(-c5c([2H])c([2H])c([2H])c([2H])c5[2H])n4)c32)c([2H])c1[2H] ; [2H]c1cc2c([2H])c(-c3nc(-c4ccc5ccccc5c4)nc(-c4cc([2H])c([2H])c5oc6c([2H])c([2H])c(-c7cccc8ccccc78)cc6c45)n3)c([2H])c([2H])c2c([2H])c1[2H] ; [2H]c1c([2H])c([2H])c(-c2nc(-c3cc4ccccc4c4ccccc34)nc(-c3cc(-c4c([2H])c([2H])c([2H])c5c4oc4c([2H])c([2H])c([2H])c([2H])c45)cc4oc5ccccc5c34)n2)c([2H])c1[2H]]

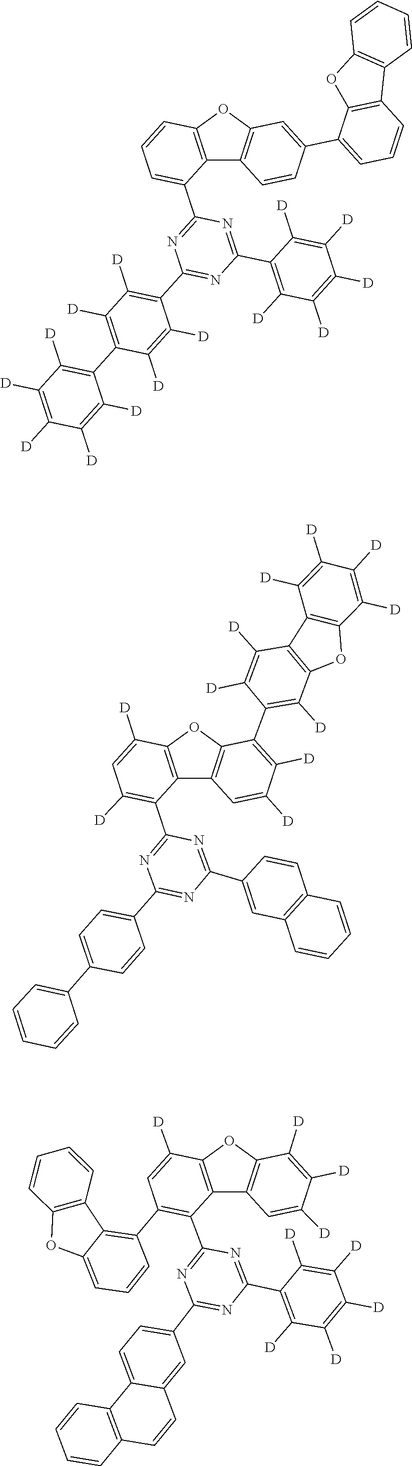 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3cccc4oc5cc(-c6cccc7c6oc6ccccc67)ccc5c34)n2)c([2H])c1[2H] ; [2H]c1cc2c(oc3c([2H])cc([2H])c(-c4nc(-c5ccc(-c6ccccc6)cc5)nc(-c5ccc6ccccc6c5)n4)c32)c(-c2c([2H])c([2H])c3c(oc4c([2H])c([2H])c([2H])c([2H])c43)c2[2H])c1[2H] ; [2H]c1cc2c(oc3c([2H])cc(-c4cccc5oc6ccccc6c45)c(-c4nc(-c5ccc6c(ccc7ccccc76)c5)nc(-c5c([2H])c([2H])c([2H])c([2H])c5[2H])n4)c32)c([2H])c1[2H]]

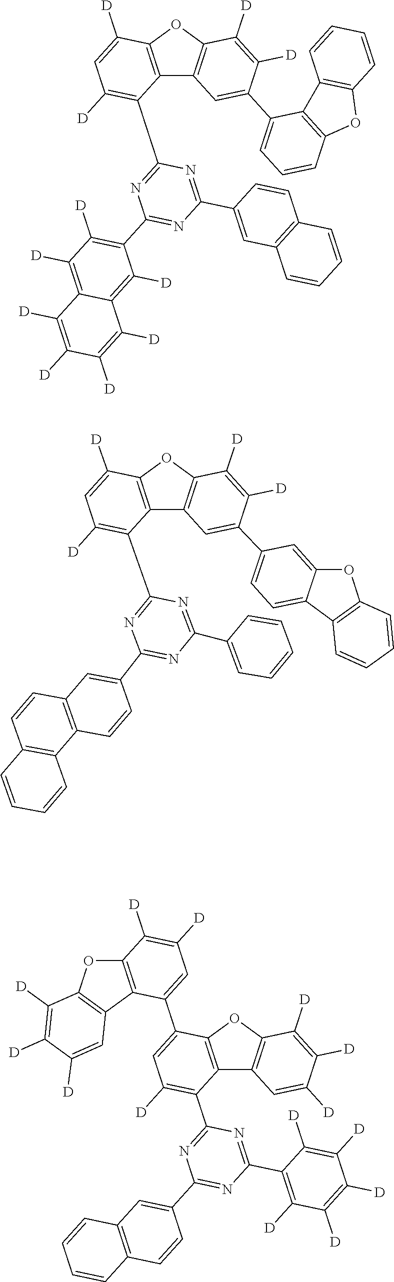 [SMILES: [2H]c1cc([2H])c2oc3c([2H])c([2H])c(-c4cccc5oc6ccccc6c45)cc3c2c1-c1nc(-c2ccc3ccccc3c2)nc(-c2c([2H])c([2H])c3c([2H])c([2H])c([2H])c([2H])c3c2[2H])n1 ; [2H]c1cc([2H])c2oc3c([2H])c([2H])c(-c4ccc5c(c4)oc4ccccc45)cc3c2c1-c1nc(-c2ccccc2)nc(-c2ccc3c(ccc4ccccc43)c2)n1 ; [2H]c1cc2c(oc3c([2H])c([2H])cc(-c4cc([2H])c(-c5nc(-c6ccc7ccccc7c6)nc(-c6c([2H])c([2H])c([2H])c([2H])c6[2H])n5)c5c4oc4c([2H])c([2H])c([2H])cc45)c32)c([2H])c1[2H]]

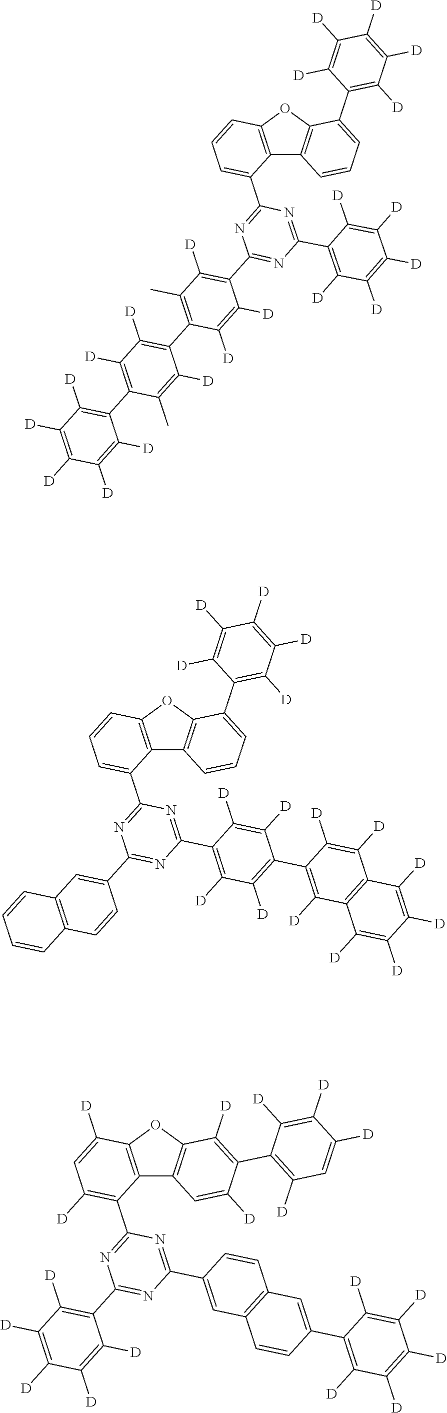

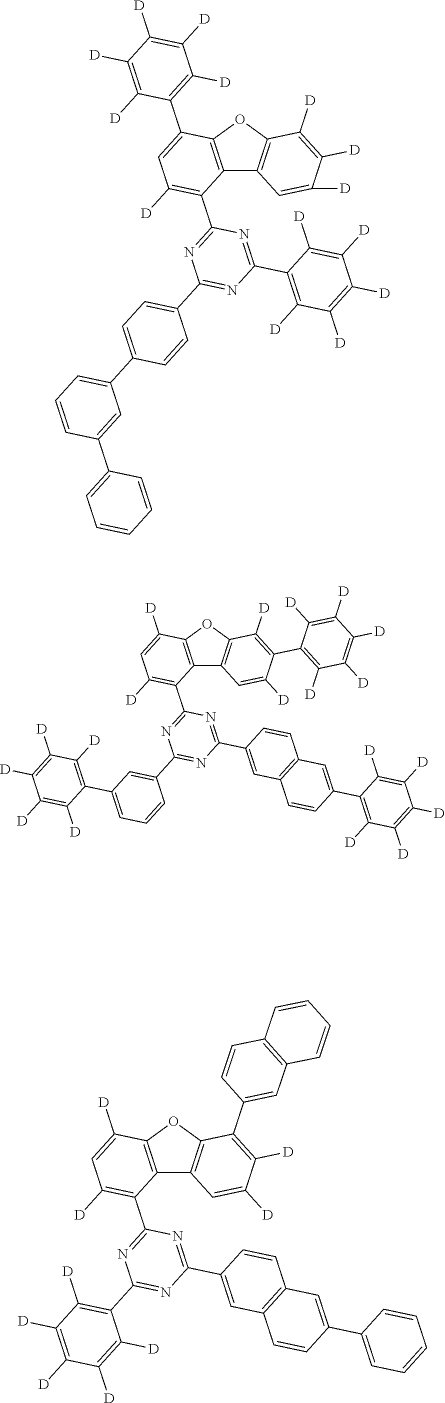

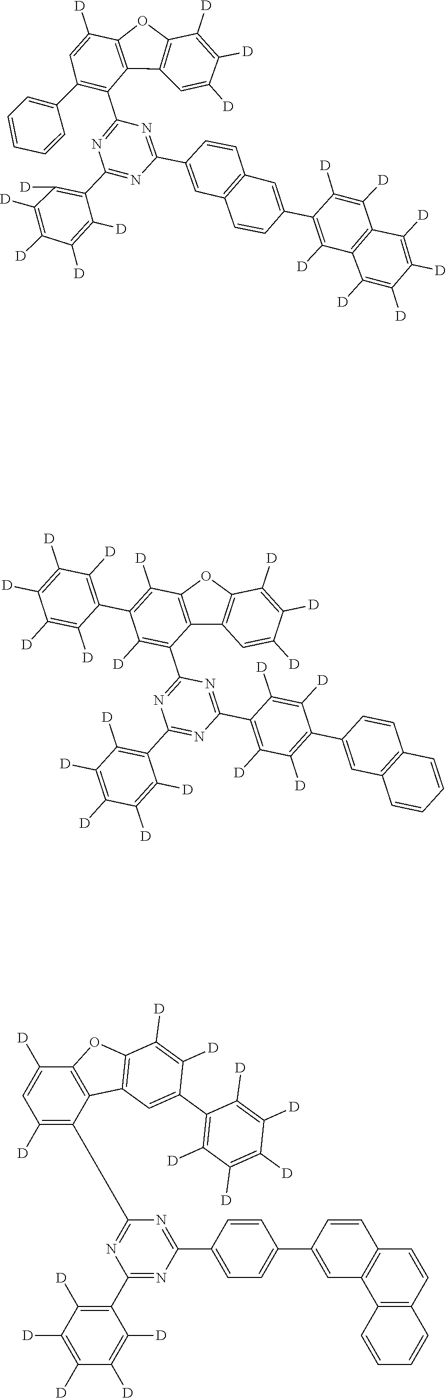

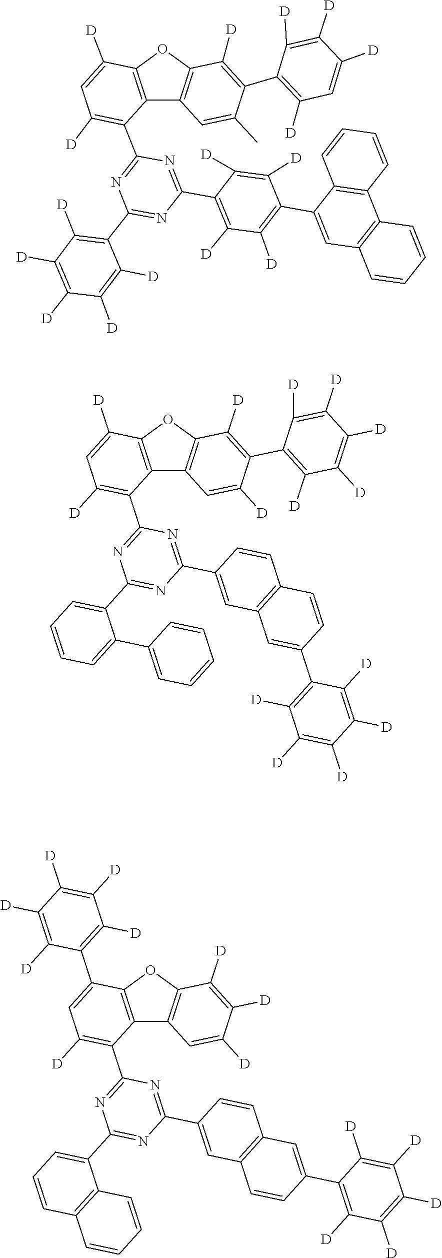

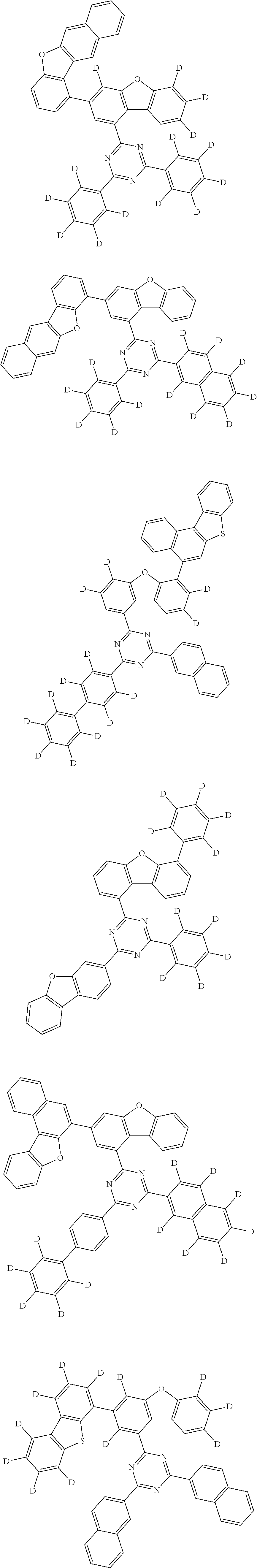

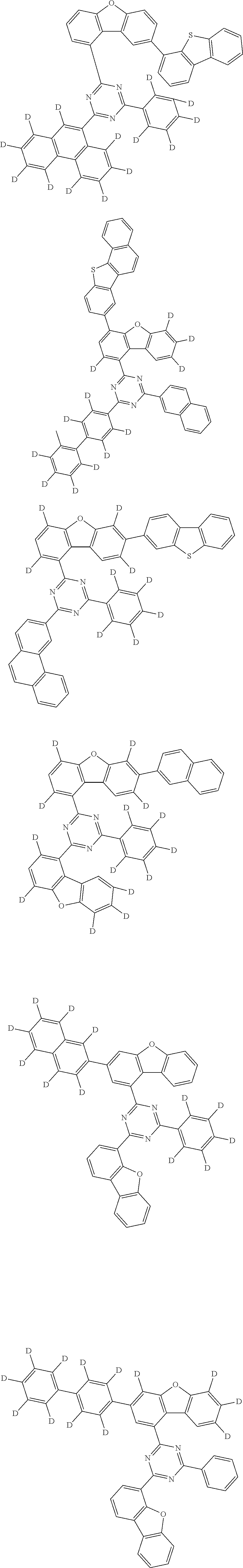

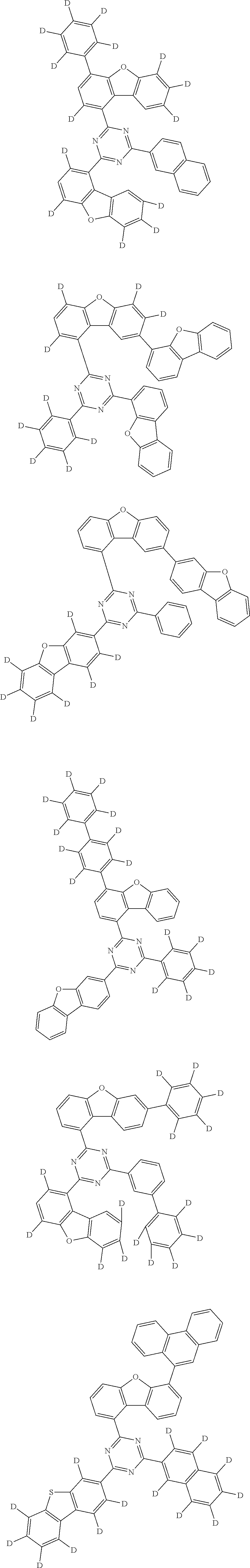

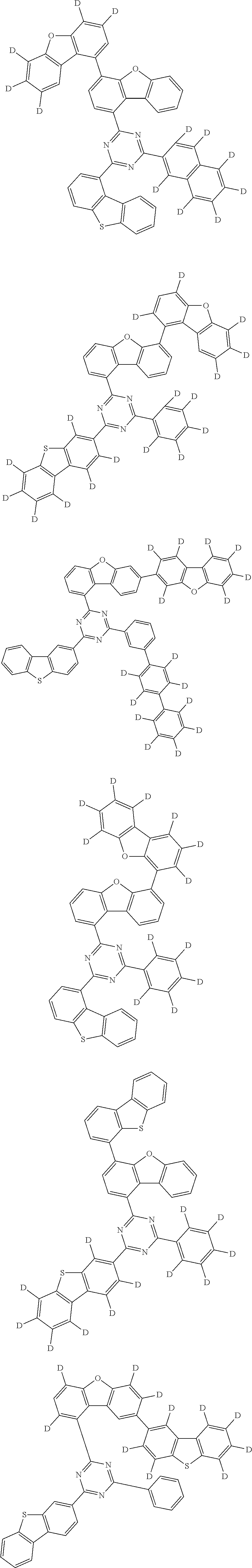

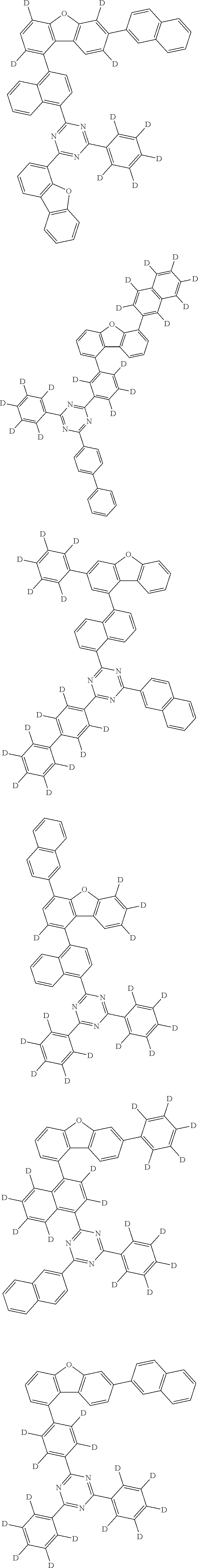

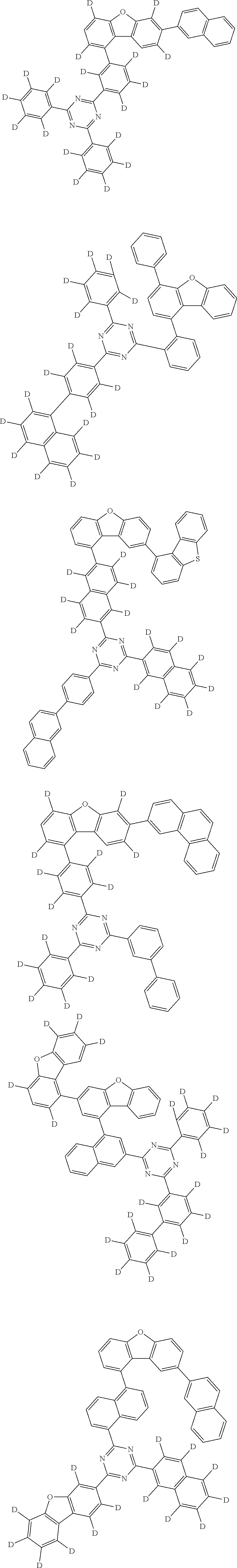

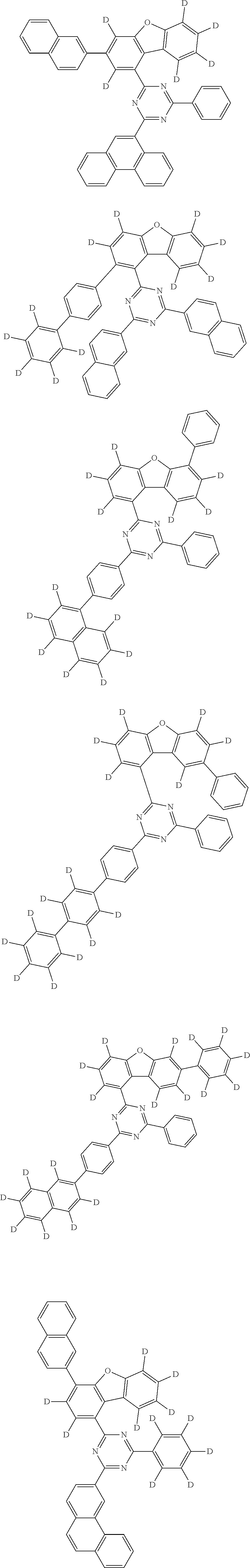

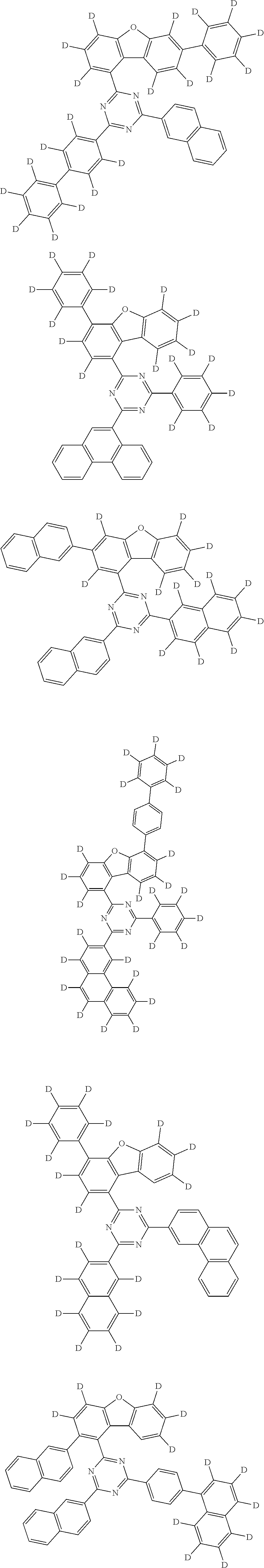

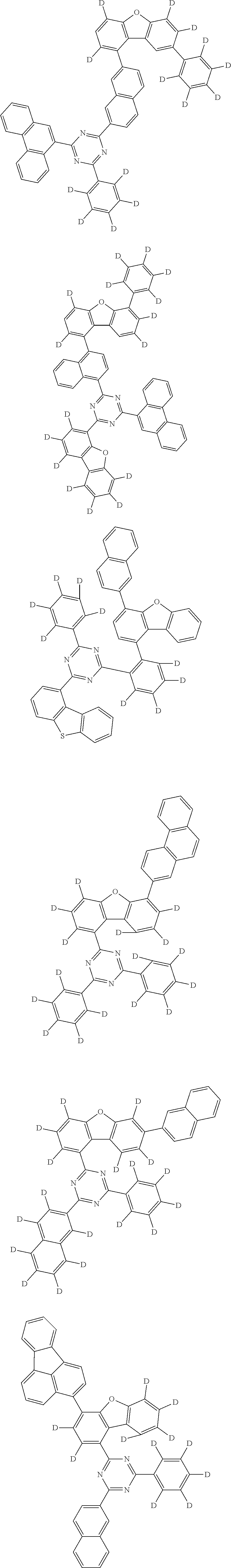

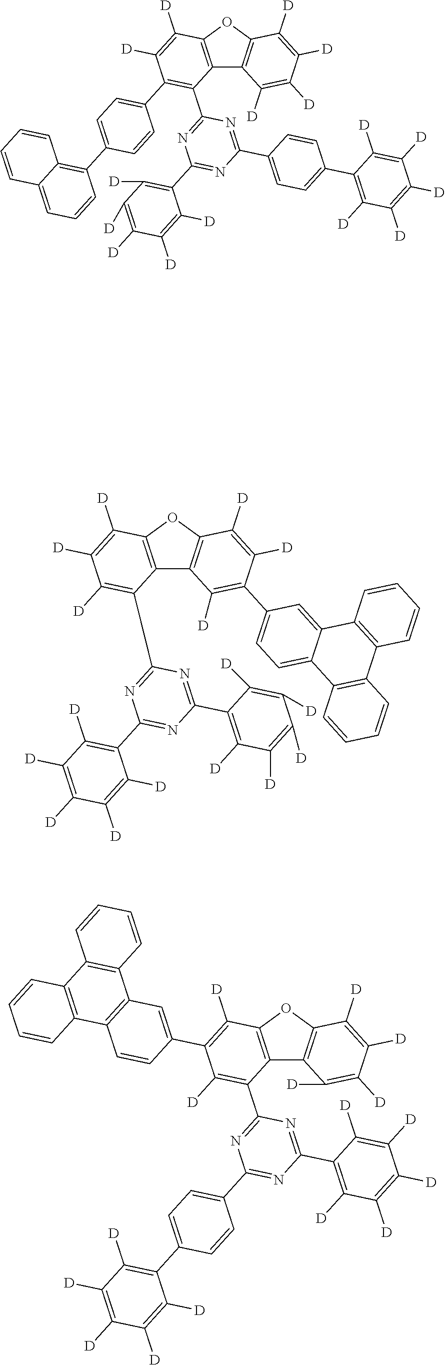

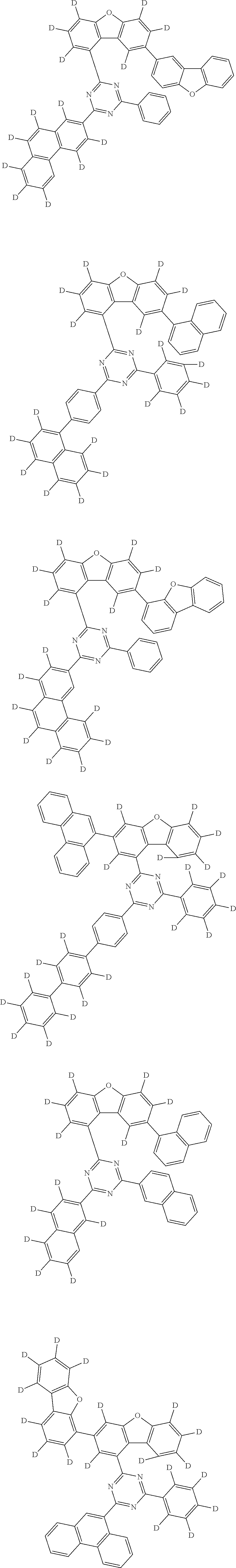

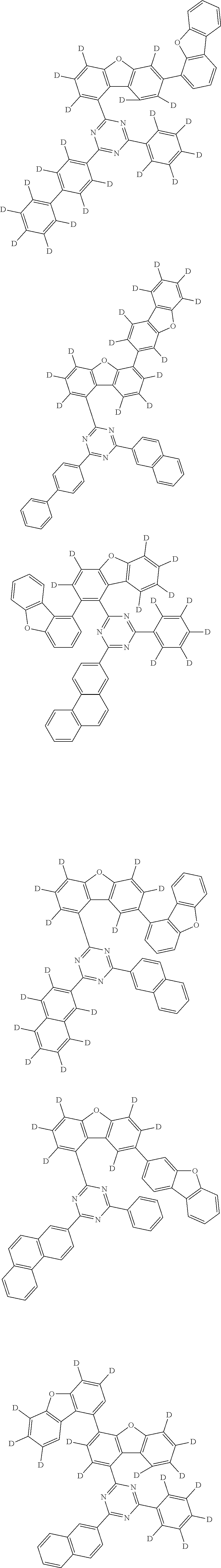

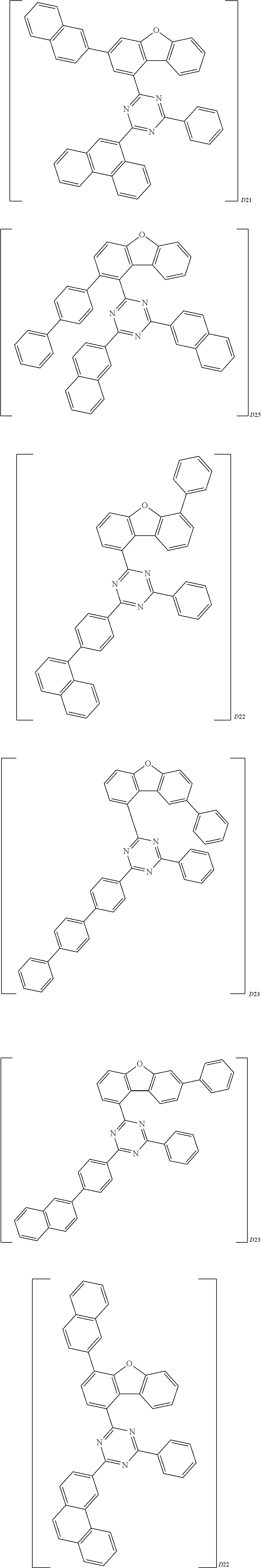

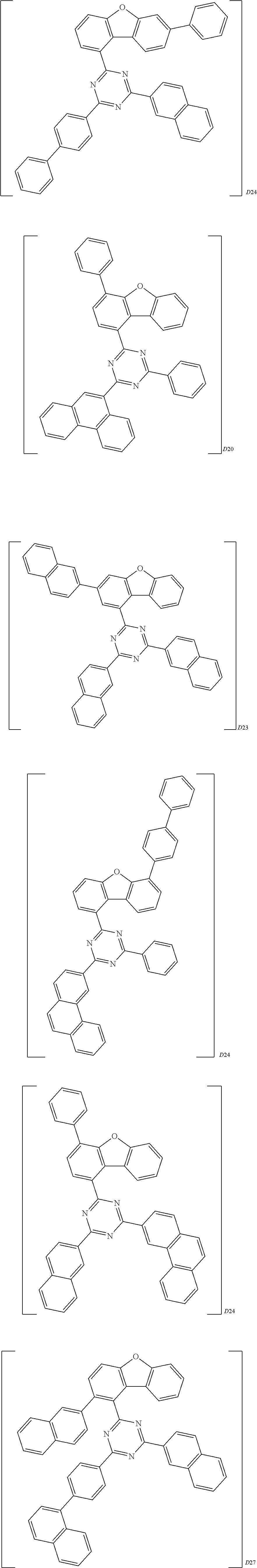

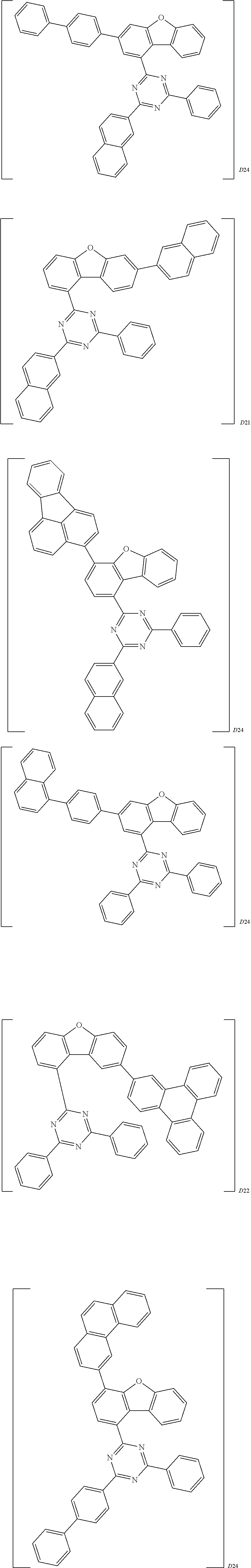

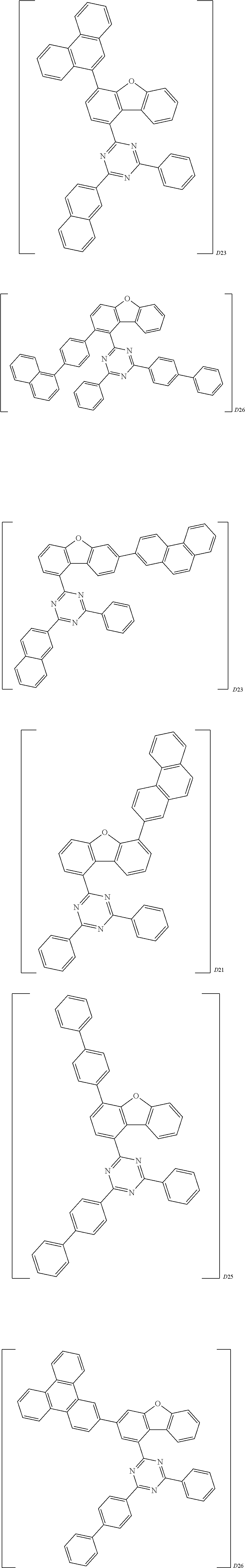

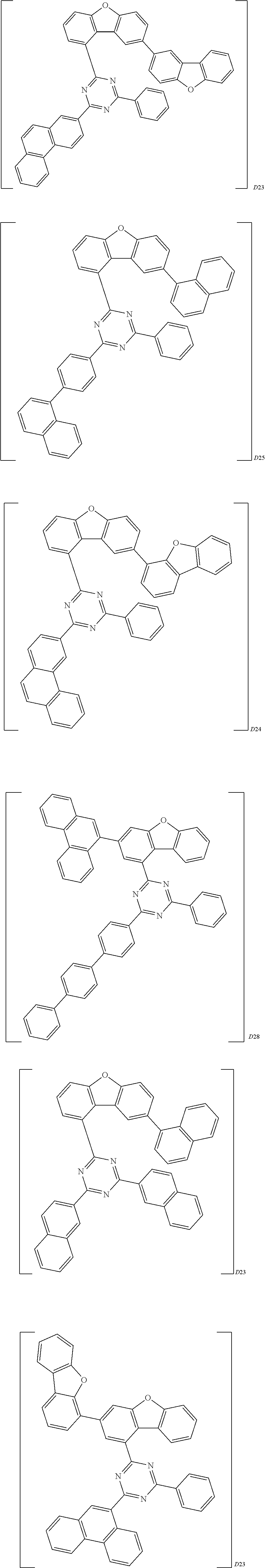

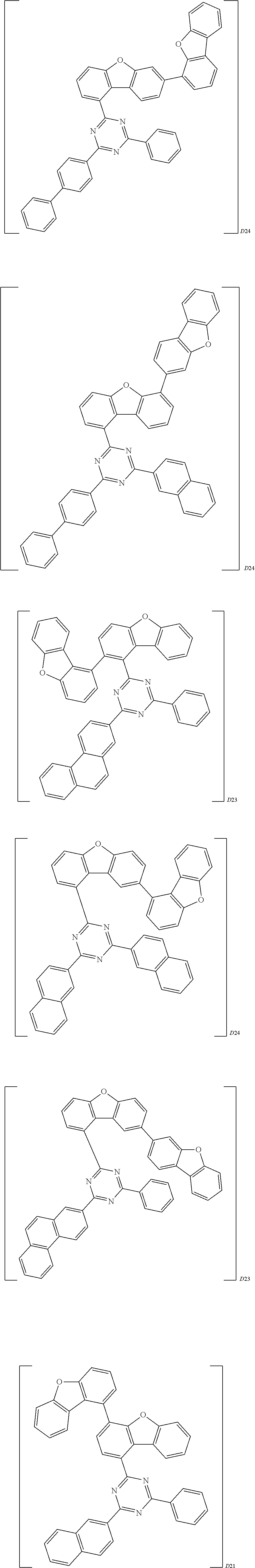

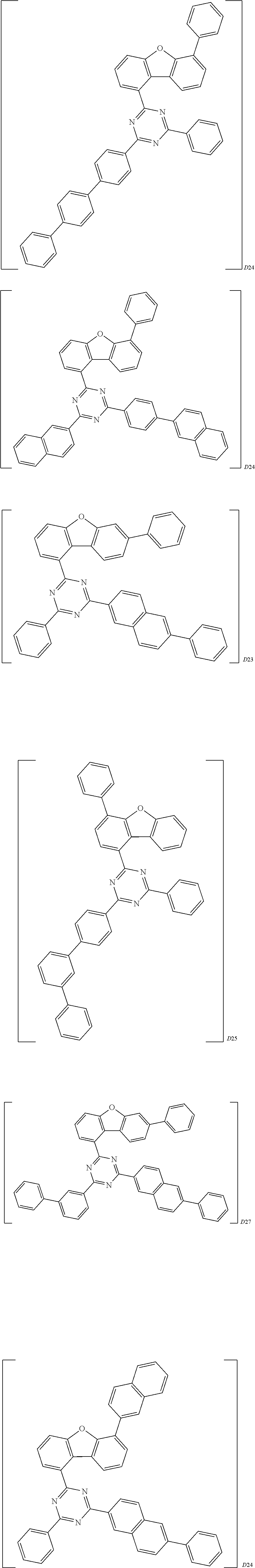

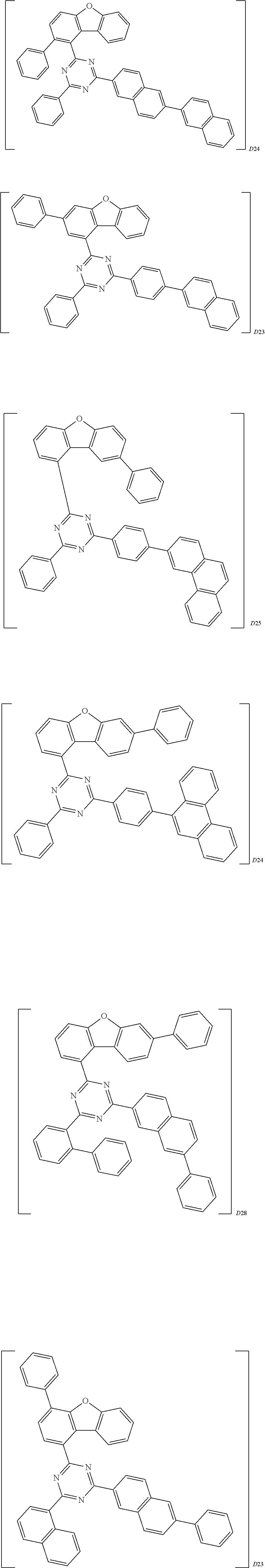

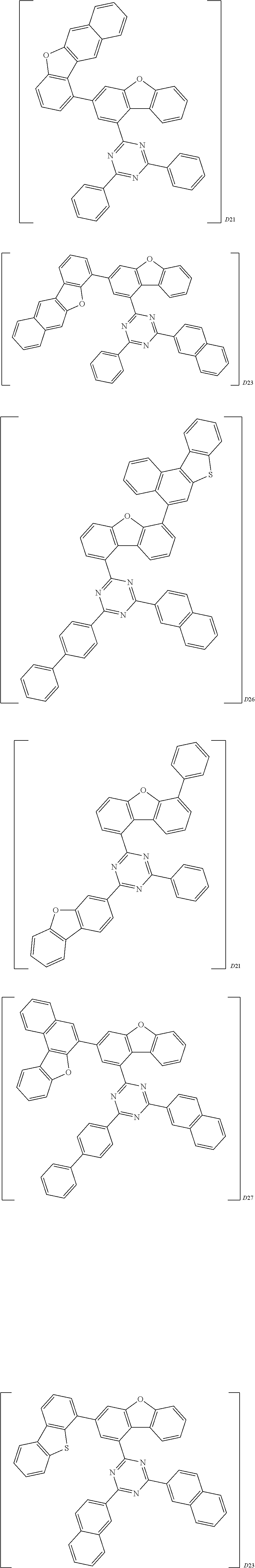

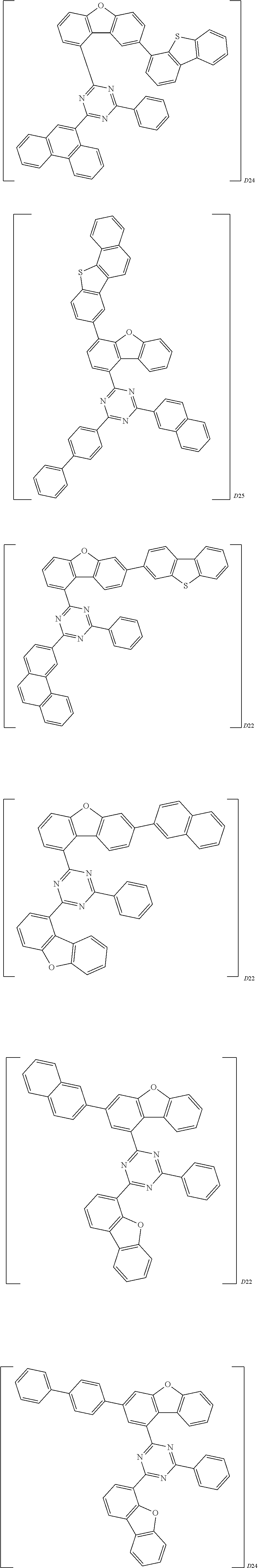

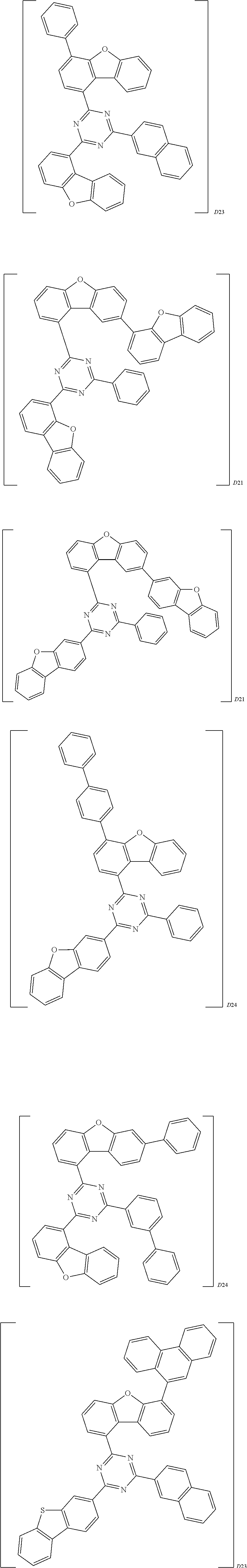

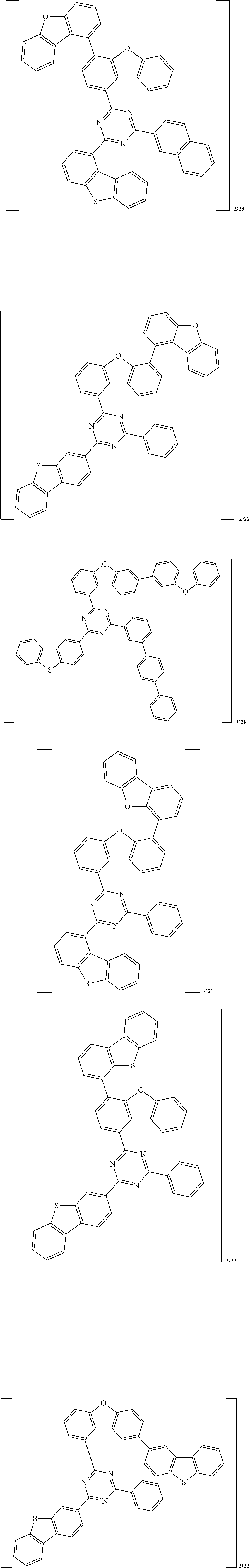

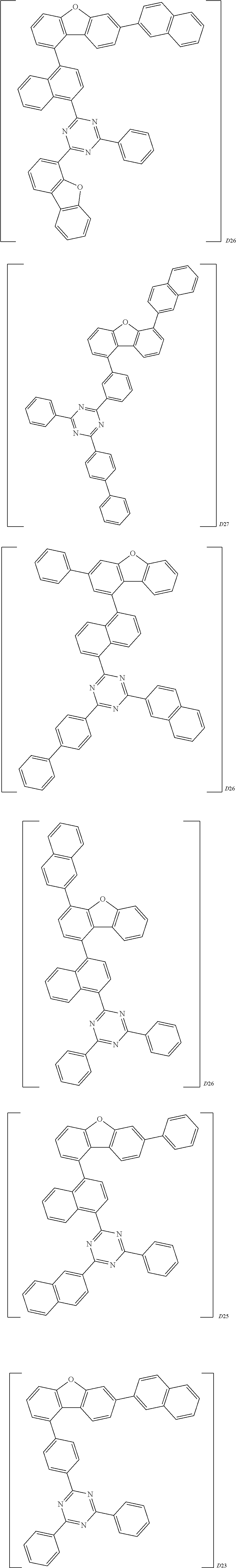

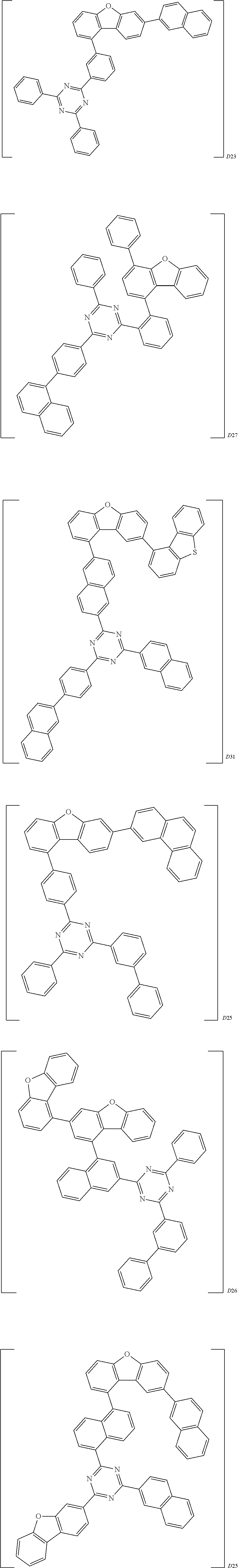

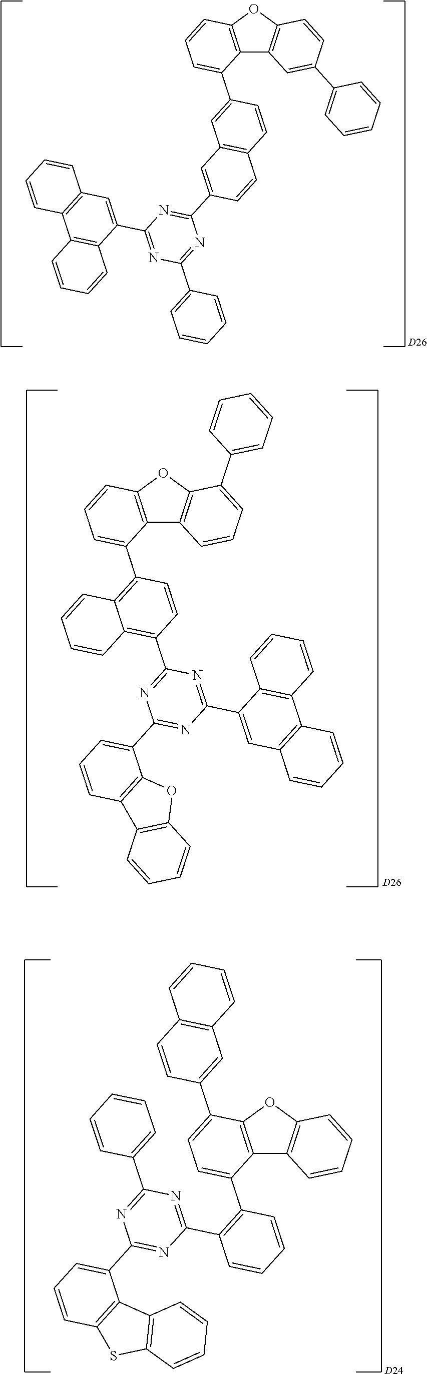

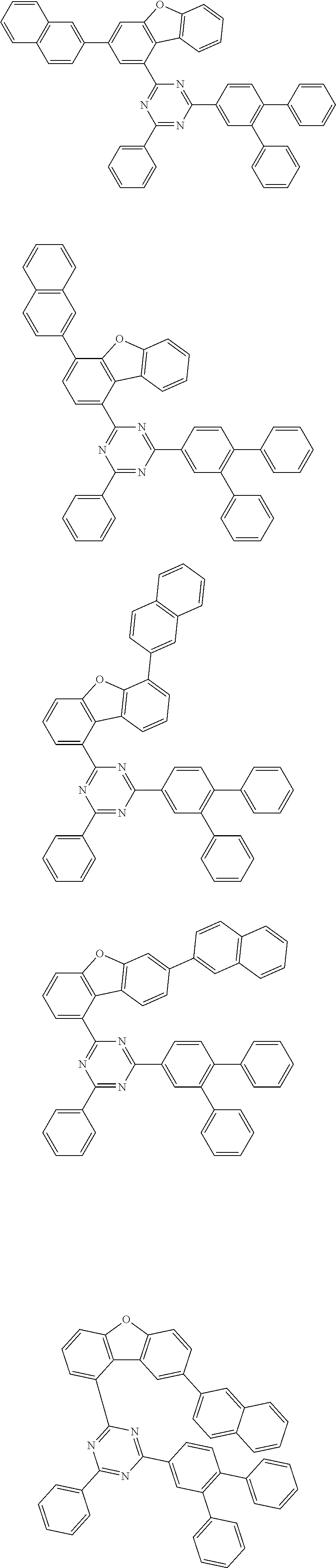 [SMILES: c1ccc(-c2nc(-c3ccc(-c4ccccc4)c(-c4ccccc4)c3)nc(-c3cc(-c4ccc5ccccc5c4)cc4oc5ccccc5c34)n2)cc1 ; c1ccc(-c2nc(-c3ccc(-c4ccccc4)c(-c4ccccc4)c3)nc(-c3ccc(-c4ccc5ccccc5c4)c4oc5ccccc5c34)n2)cc1 ; c1ccc(-c2nc(-c3ccc(-c4ccccc4)c(-c4ccccc4)c3)nc(-c3cccc4oc5c(-c6ccc7ccccc7c6)cccc5c34)n2)cc1 ; c1ccc(-c2nc(-c3ccc(-c4ccccc4)c(-c4ccccc4)c3)nc(-c3cccc4oc5cc(-c6ccc7ccccc7c6)ccc5c34)n2)cc1 ; c1ccc(-c2nc(-c3ccc(-c4ccccc4)c(-c4ccccc4)c3)nc(-c3cccc4oc5ccc(-c6ccc7ccccc7c6)cc5c34)n2)cc1]

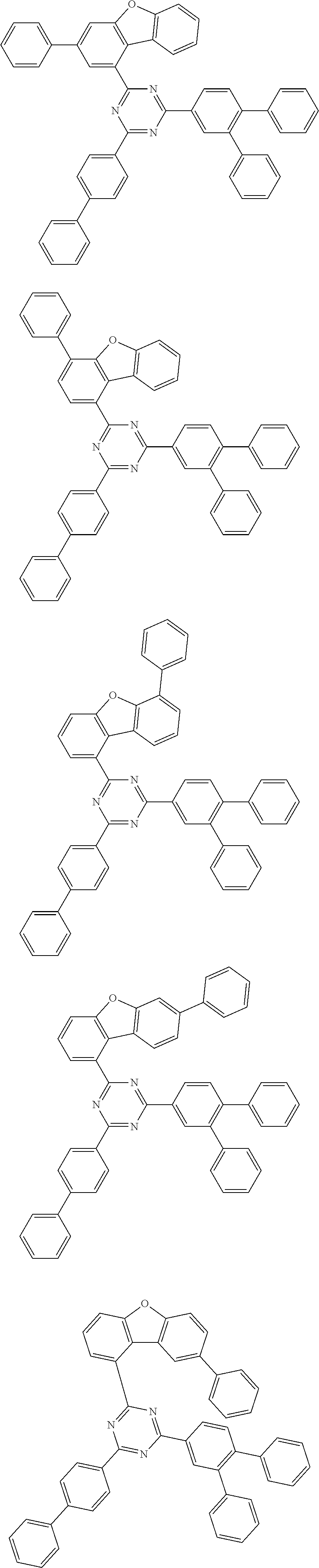

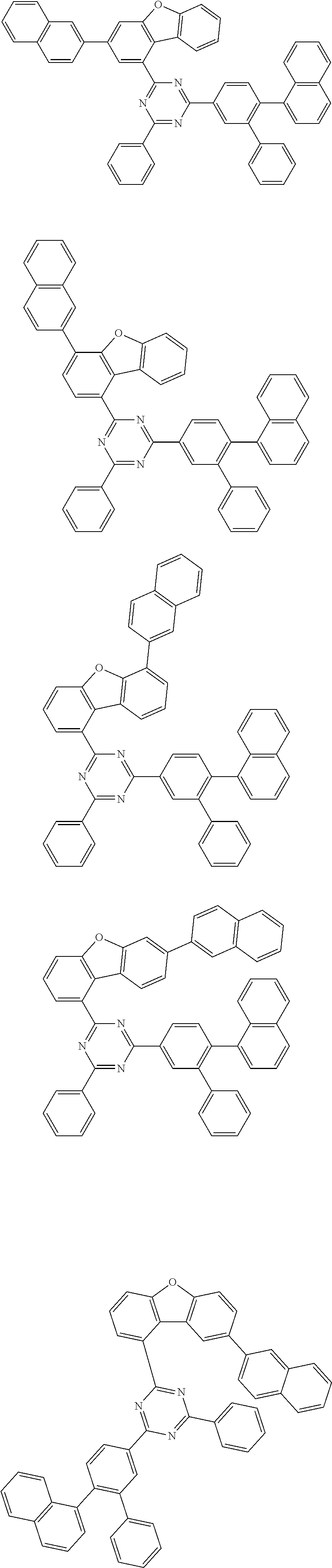 [SMILES: c1ccc(-c2nc(-c3ccc(-c4cccc5ccccc45)c(-c4ccccc4)c3)nc(-c3cc(-c4ccc5ccccc5c4)cc4oc5ccccc5c34)n2)cc1 ; c1ccc(-c2nc(-c3ccc(-c4cccc5ccccc45)c(-c4ccccc4)c3)nc(-c3ccc(-c4ccc5ccccc5c4)c4oc5ccccc5c34)n2)cc1 ; c1ccc(-c2nc(-c3ccc(-c4cccc5ccccc45)c(-c4ccccc4)c3)nc(-c3cccc4oc5c(-c6ccc7ccccc7c6)cccc5c34)n2)cc1 ; c1ccc(-c2nc(-c3ccc(-c4cccc5ccccc45)c(-c4ccccc4)c3)nc(-c3cccc4oc5cc(-c6ccc7ccccc7c6)ccc5c34)n2)cc1 ; c1ccc(-c2nc(-c3ccc(-c4cccc5ccccc45)c(-c4ccccc4)c3)nc(-c3cccc4oc5ccc(-c6ccc7ccccc7c6)cc5c34)n2)cc1]

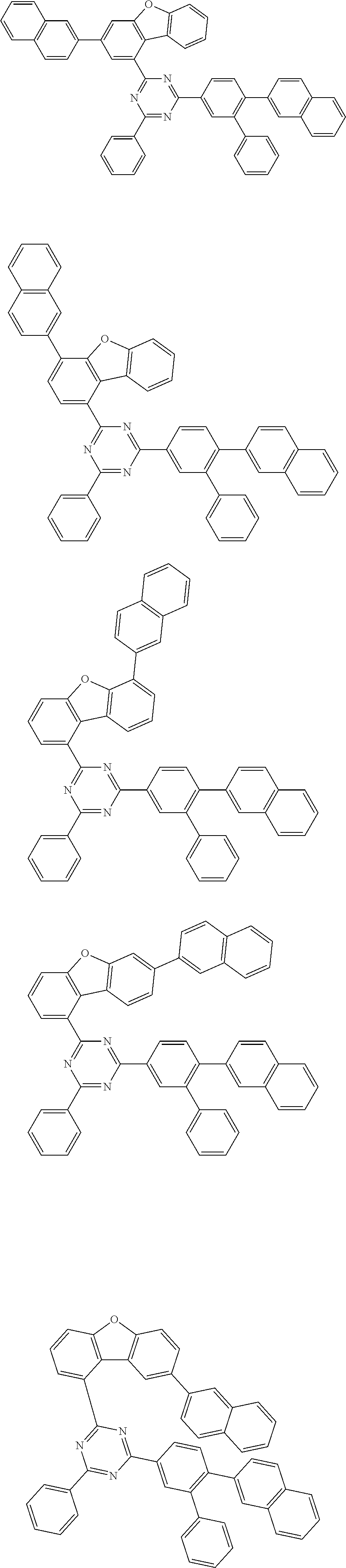 [SMILES: c1ccc(-c2nc(-c3ccc(-c4ccc5ccccc5c4)c(-c4ccccc4)c3)nc(-c3cc(-c4ccc5ccccc5c4)cc4oc5ccccc5c34)n2)cc1 ; c1ccc(-c2nc(-c3ccc(-c4ccc5ccccc5c4)c(-c4ccccc4)c3)nc(-c3ccc(-c4ccc5ccccc5c4)c4oc5ccccc5c34)n2)cc1 ; c1ccc(-c2nc(-c3ccc(-c4ccc5ccccc5c4)c(-c4ccccc4)c3)nc(-c3cccc4oc5c(-c6ccc7ccccc7c6)cccc5c34)n2)cc1 ; c1ccc(-c2nc(-c3ccc(-c4ccc5ccccc5c4)c(-c4ccccc4)c3)nc(-c3cccc4oc5cc(-c6ccc7ccccc7c6)ccc5c34)n2)cc1 ; c1ccc(-c2nc(-c3ccc(-c4ccc5ccccc5c4)c(-c4ccccc4)c3)nc(-c3cccc4oc5ccc(-c6ccc7ccccc7c6)cc5c34)n2)cc1]

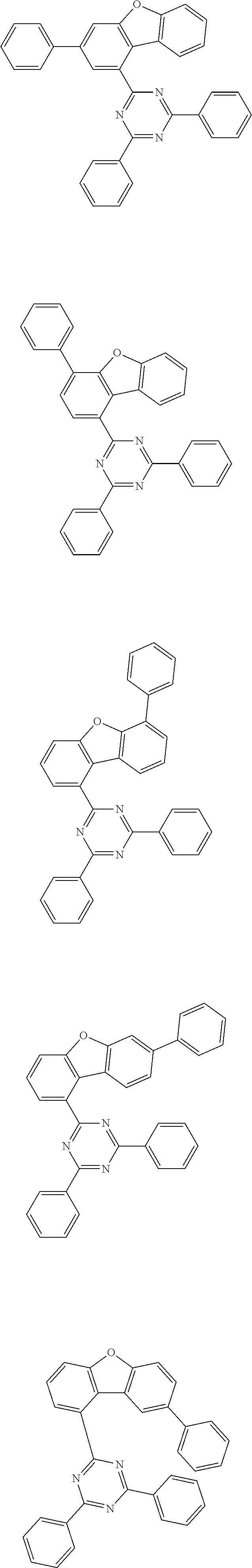 [SMILES: c1ccc(-c2cc(-c3nc(-c4ccccc4)nc(-c4ccccc4)n3)c3c(c2)oc2ccccc23)cc1 ; c1ccc(-c2nc(-c3ccccc3)nc(-c3ccc(-c4ccccc4)c4oc5ccccc5c34)n2)cc1 ; c1ccc(-c2nc(-c3ccccc3)nc(-c3cccc4oc5c(-c6ccccc6)cccc5c34)n2)cc1 ; c1ccc(-c2ccc3c(c2)oc2cccc(-c4nc(-c5ccccc5)nc(-c5ccccc5)n4)c23)cc1 ; c1ccc(-c2ccc3oc4cccc(-c5nc(-c6ccccc6)nc(-c6ccccc6)n5)c4c3c2)cc1]

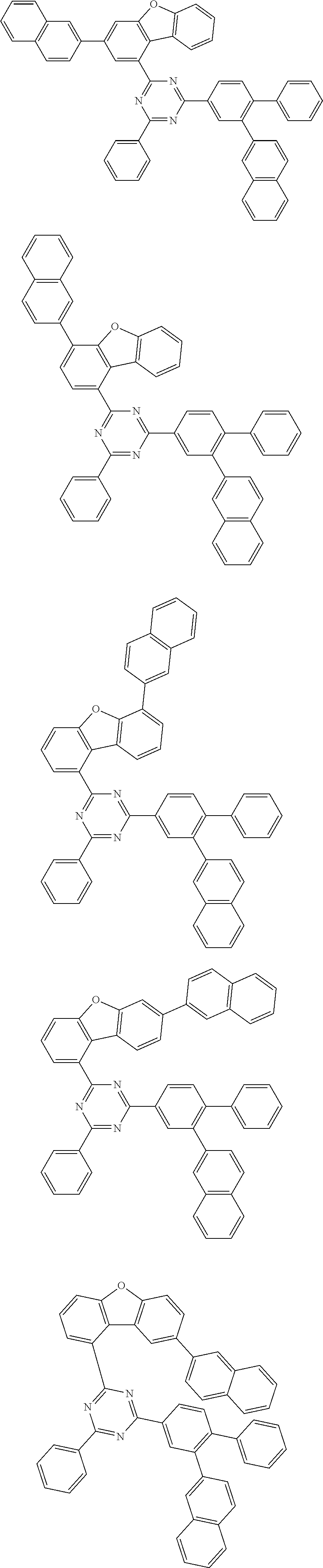

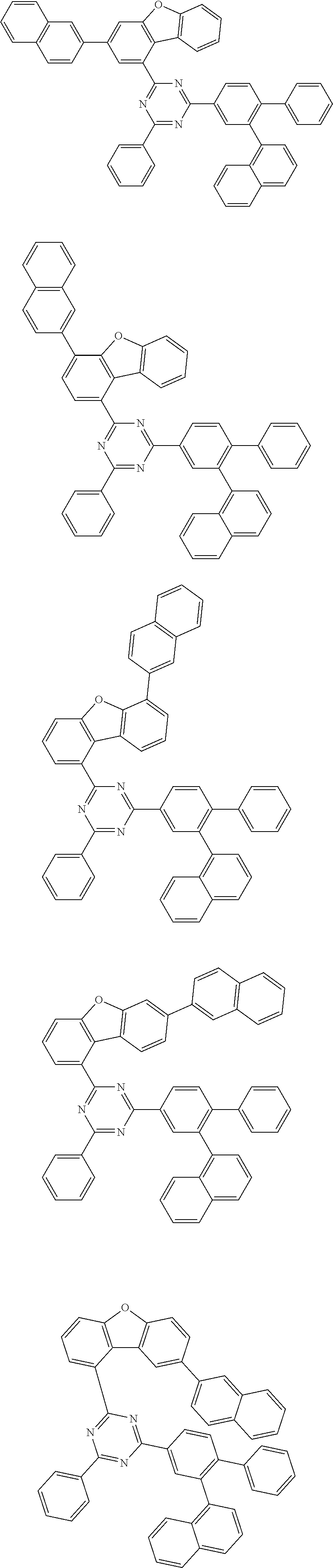

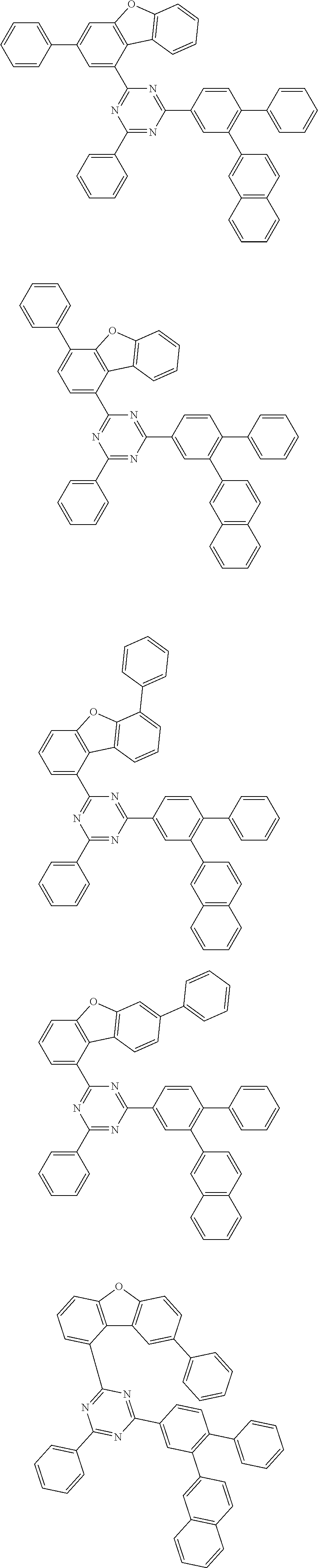

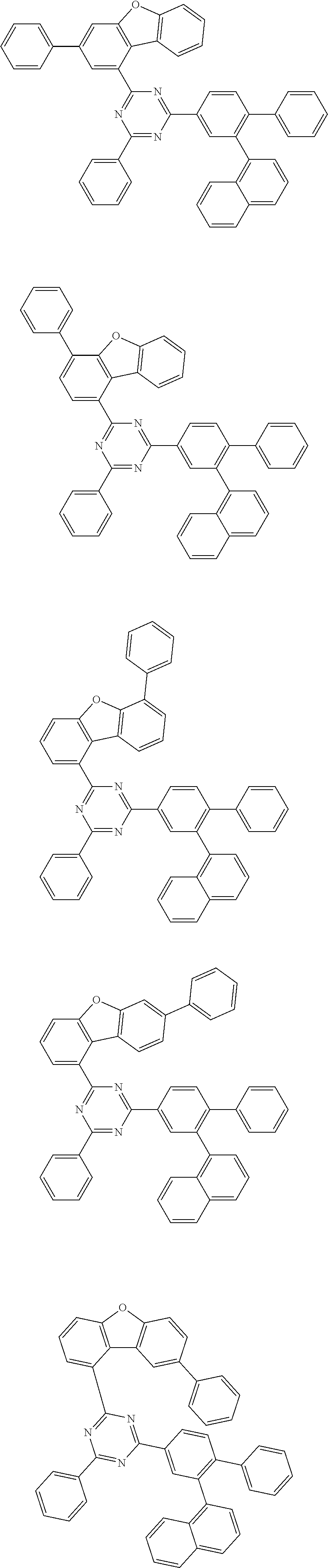 [SMILES: c1ccc(-c2cc(-c3nc(-c4ccccc4)nc(-c4ccc(-c5ccccc5)c(-c5cccc6ccccc56)c4)n3)c3c(c2)oc2ccccc23)cc1 ; c1ccc(-c2nc(-c3ccc(-c4ccccc4)c(-c4cccc5ccccc45)c3)nc(-c3ccc(-c4ccccc4)c4oc5ccccc5c34)n2)cc1 ; c1ccc(-c2nc(-c3ccc(-c4ccccc4)c(-c4cccc5ccccc45)c3)nc(-c3cccc4oc5c(-c6ccccc6)cccc5c34)n2)cc1 ; c1ccc(-c2ccc3c(c2)oc2cccc(-c4nc(-c5ccccc5)nc(-c5ccc(-c6ccccc6)c(-c6cccc7ccccc67)c5)n4)c23)cc1 ; c1ccc(-c2ccc3oc4cccc(-c5nc(-c6ccccc6)nc(-c6ccc(-c7ccccc7)c(-c7cccc8ccccc78)c6)n5)c4c3c2)cc1]

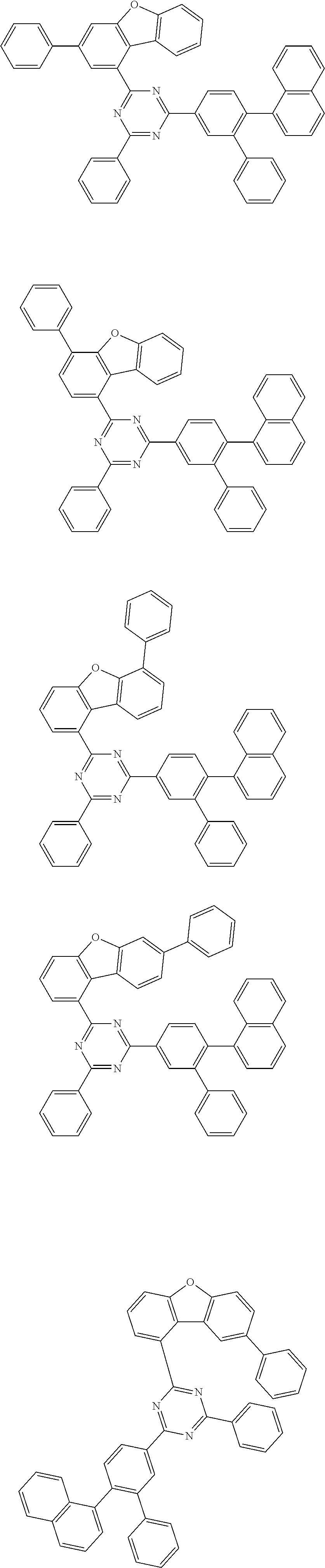 [SMILES: c1ccc(-c2cc(-c3nc(-c4ccccc4)nc(-c4ccc(-c5cccc6ccccc56)c(-c5ccccc5)c4)n3)c3c(c2)oc2ccccc23)cc1 ; c1ccc(-c2nc(-c3ccc(-c4cccc5ccccc45)c(-c4ccccc4)c3)nc(-c3ccc(-c4ccccc4)c4oc5ccccc5c34)n2)cc1 ; c1ccc(-c2nc(-c3ccc(-c4cccc5ccccc45)c(-c4ccccc4)c3)nc(-c3cccc4oc5c(-c6ccccc6)cccc5c34)n2)cc1 ; c1ccc(-c2ccc3c(c2)oc2cccc(-c4nc(-c5ccccc5)nc(-c5ccc(-c6cccc7ccccc67)c(-c6ccccc6)c5)n4)c23)cc1 ; c1ccc(-c2ccc3oc4cccc(-c5nc(-c6ccccc6)nc(-c6ccc(-c7cccc8ccccc78)c(-c7ccccc7)c6)n5)c4c3c2)cc1]

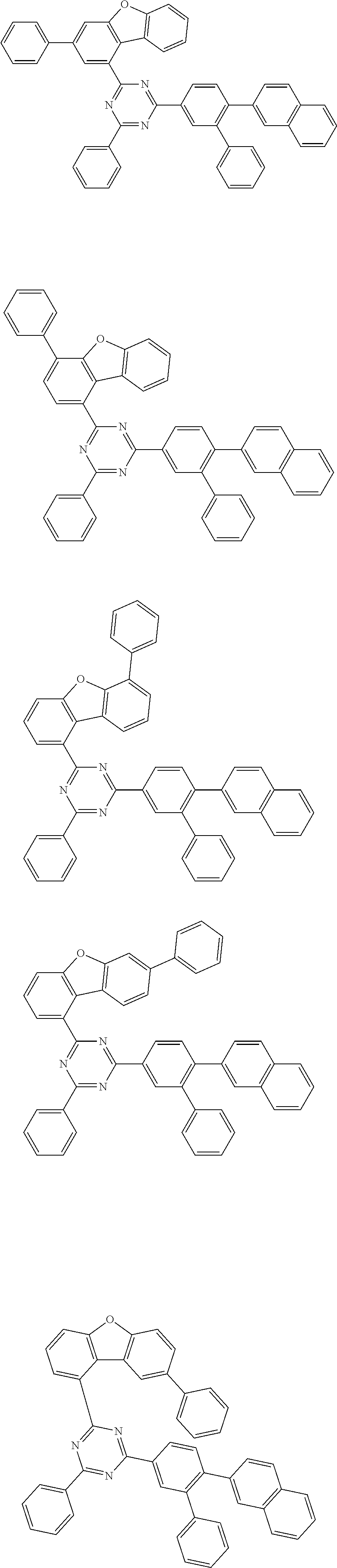 [SMILES: c1ccc(-c2cc(-c3nc(-c4ccccc4)nc(-c4ccc(-c5ccc6ccccc6c5)c(-c5ccccc5)c4)n3)c3c(c2)oc2ccccc23)cc1 ; c1ccc(-c2nc(-c3ccc(-c4ccc5ccccc5c4)c(-c4ccccc4)c3)nc(-c3ccc(-c4ccccc4)c4oc5ccccc5c34)n2)cc1 ; c1ccc(-c2nc(-c3ccc(-c4ccc5ccccc5c4)c(-c4ccccc4)c3)nc(-c3cccc4oc5c(-c6ccccc6)cccc5c34)n2)cc1 ; c1ccc(-c2ccc3c(c2)oc2cccc(-c4nc(-c5ccccc5)nc(-c5ccc(-c6ccc7ccccc7c6)c(-c6ccccc6)c5)n4)c23)cc1 ; c1ccc(-c2ccc3oc4cccc(-c5nc(-c6ccccc6)nc(-c6ccc(-c7ccc8ccccc8c7)c(-c7ccccc7)c6)n5)c4c3c2)cc1]

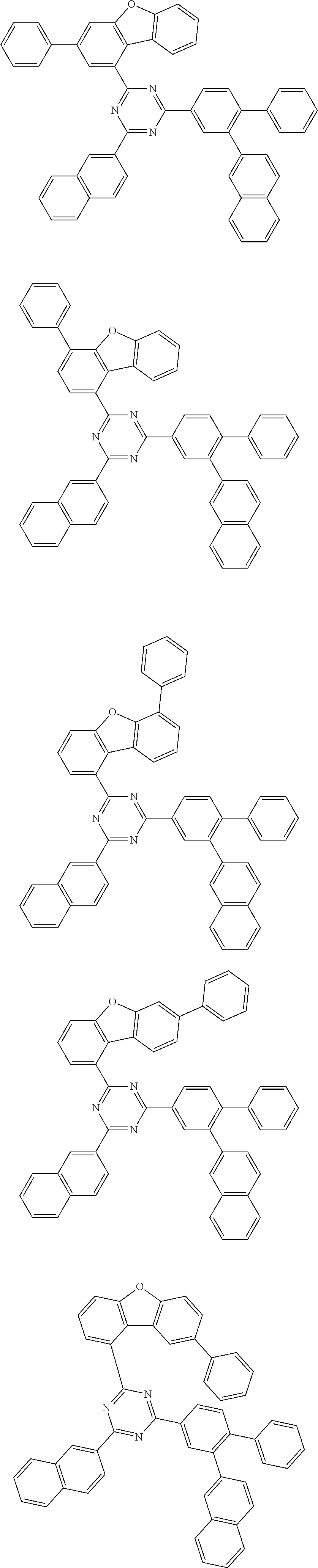

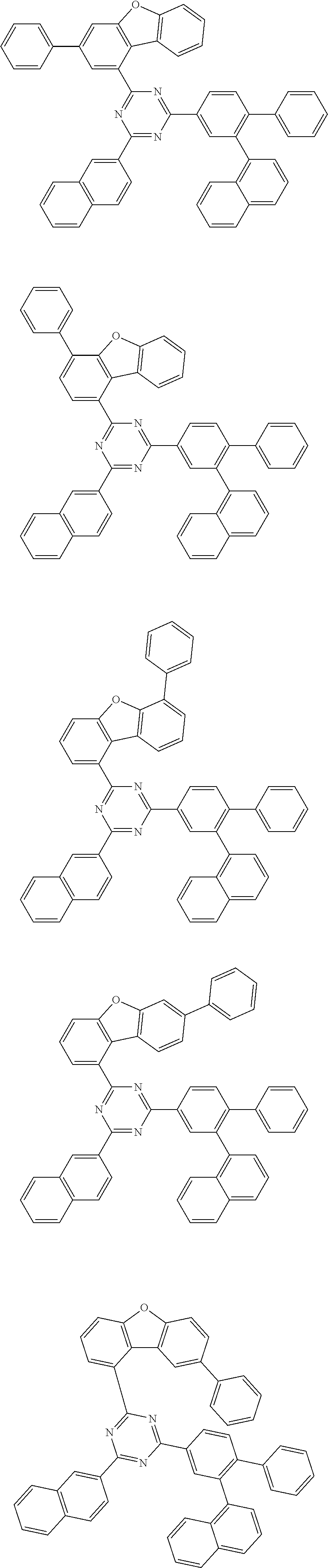 [SMILES: c1ccc(-c2cc(-c3nc(-c4ccc(-c5ccccc5)c(-c5cccc6ccccc56)c4)nc(-c4ccc5ccccc5c4)n3)c3c(c2)oc2ccccc23)cc1 ; c1ccc(-c2ccc(-c3nc(-c4ccc5ccccc5c4)nc(-c4ccc(-c5ccccc5)c5oc6ccccc6c45)n3)cc2-c2cccc3ccccc23)cc1 ; c1ccc(-c2ccc(-c3nc(-c4ccc5ccccc5c4)nc(-c4cccc5oc6c(-c7ccccc7)cccc6c45)n3)cc2-c2cccc3ccccc23)cc1 ; c1ccc(-c2ccc3c(c2)oc2cccc(-c4nc(-c5ccc(-c6ccccc6)c(-c6cccc7ccccc67)c5)nc(-c5ccc6ccccc6c5)n4)c23)cc1 ; c1ccc(-c2ccc3oc4cccc(-c5nc(-c6ccc(-c7ccccc7)c(-c7cccc8ccccc78)c6)nc(-c6ccc7ccccc7c6)n5)c4c3c2)cc1]

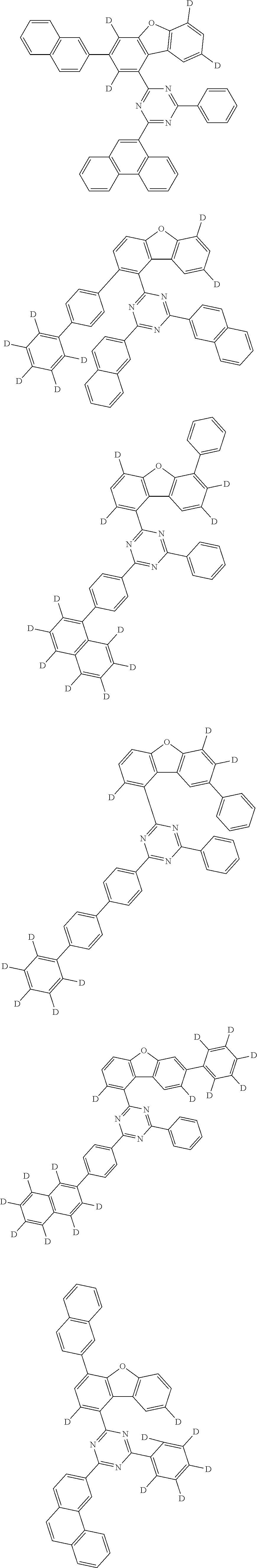

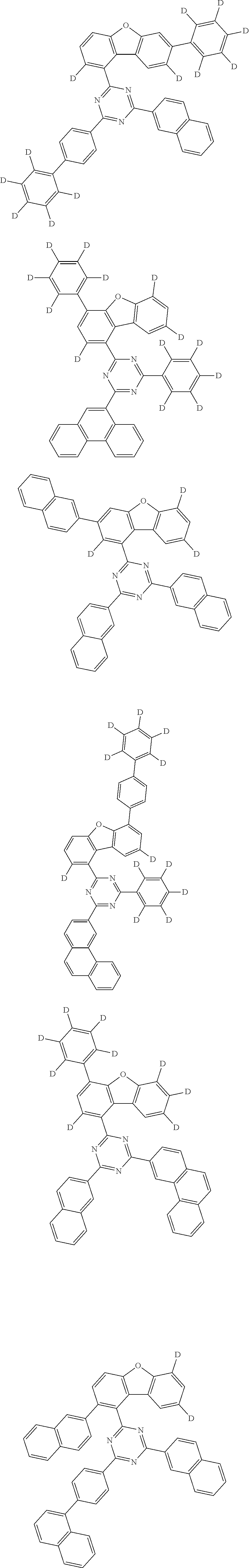

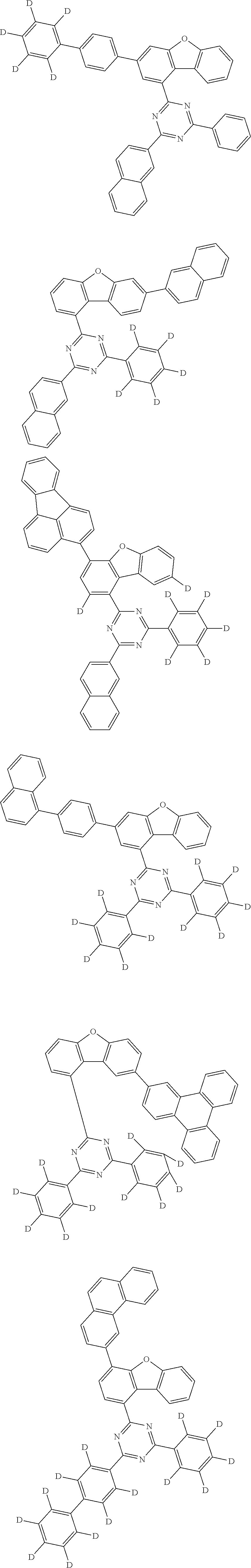

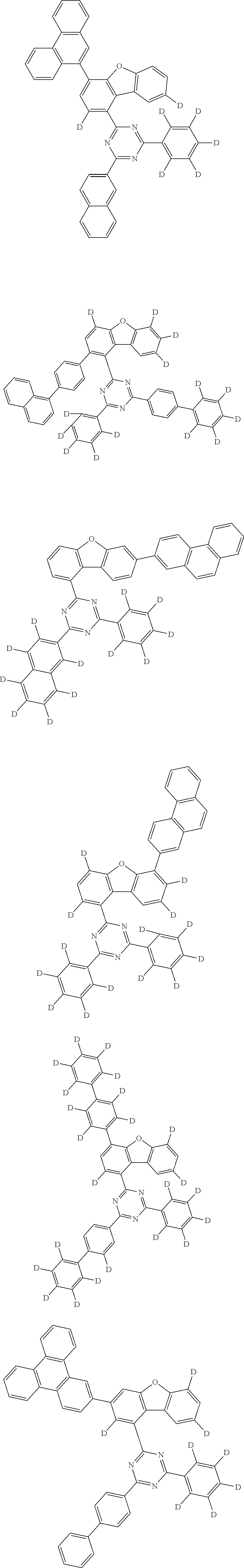

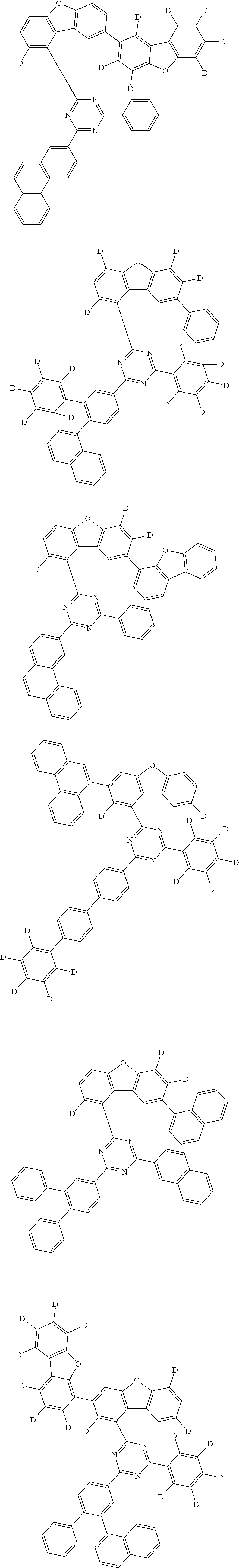

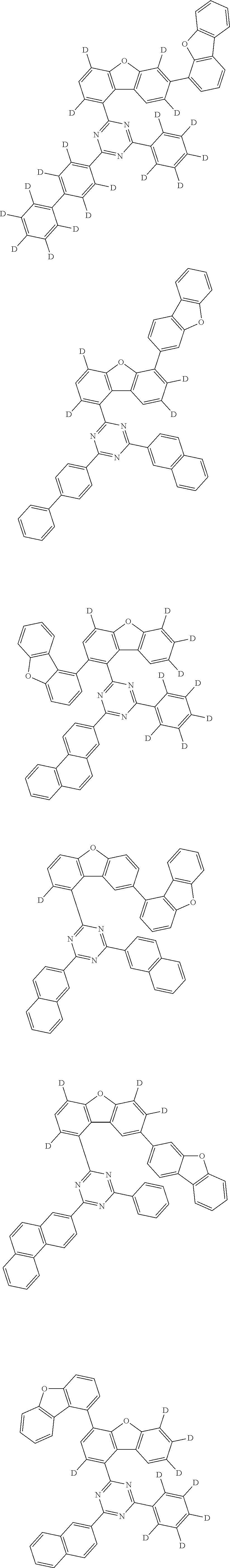

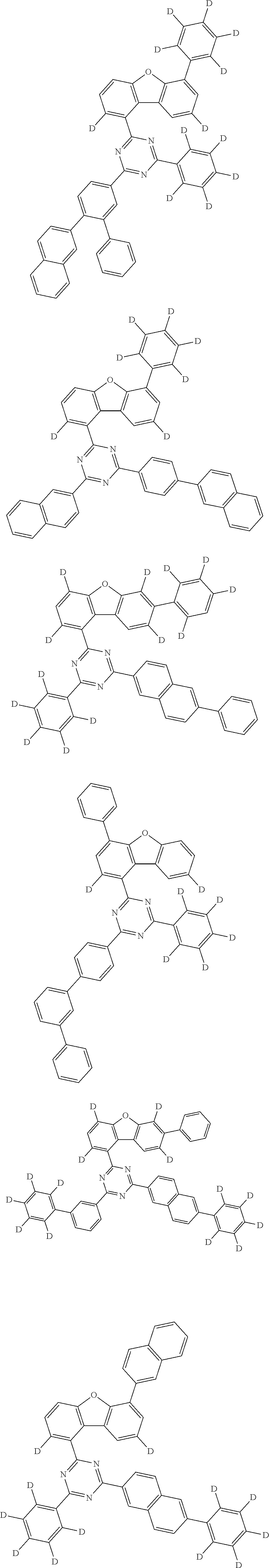

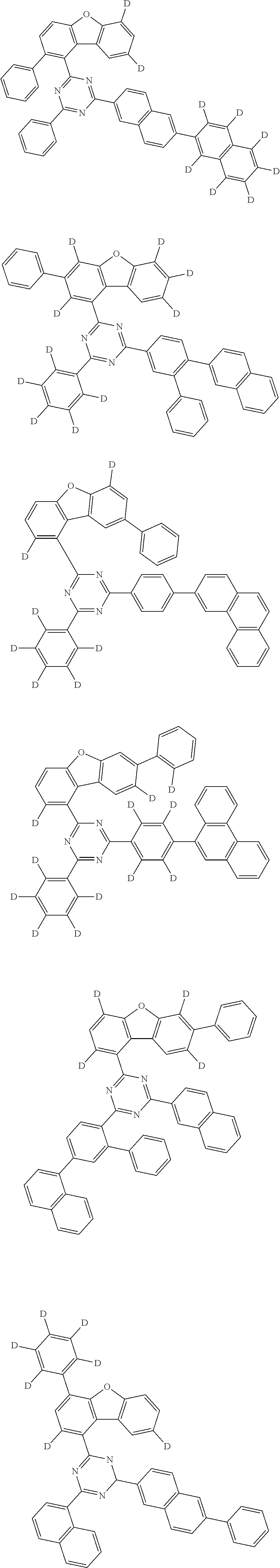

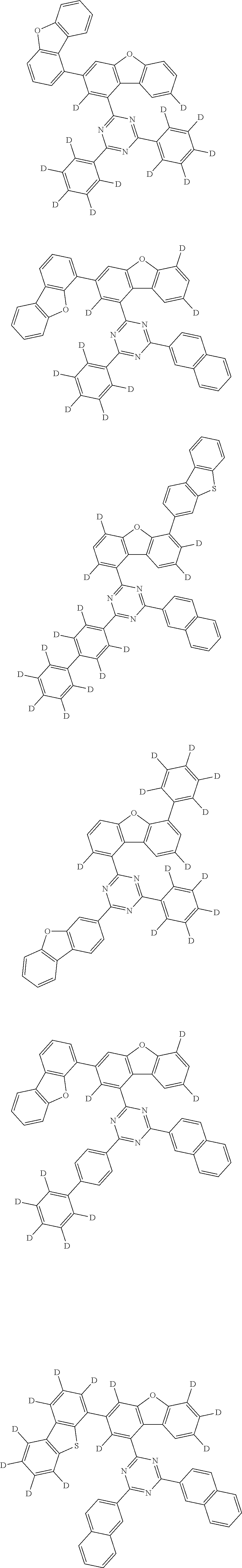

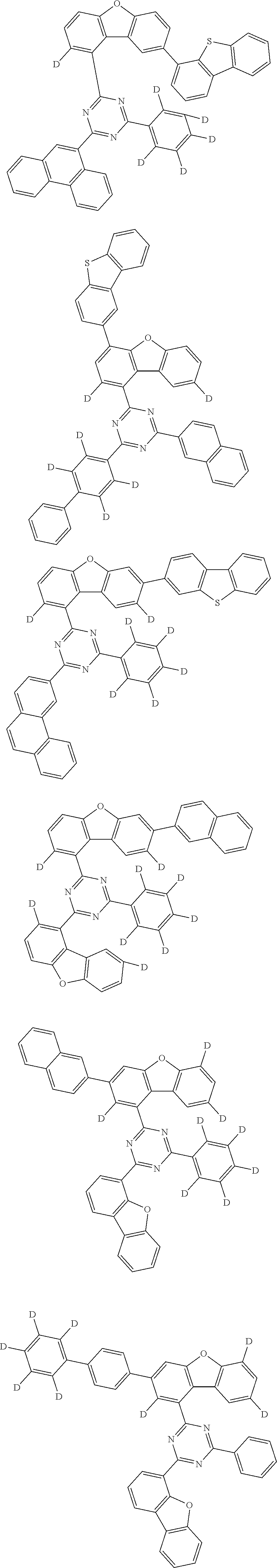

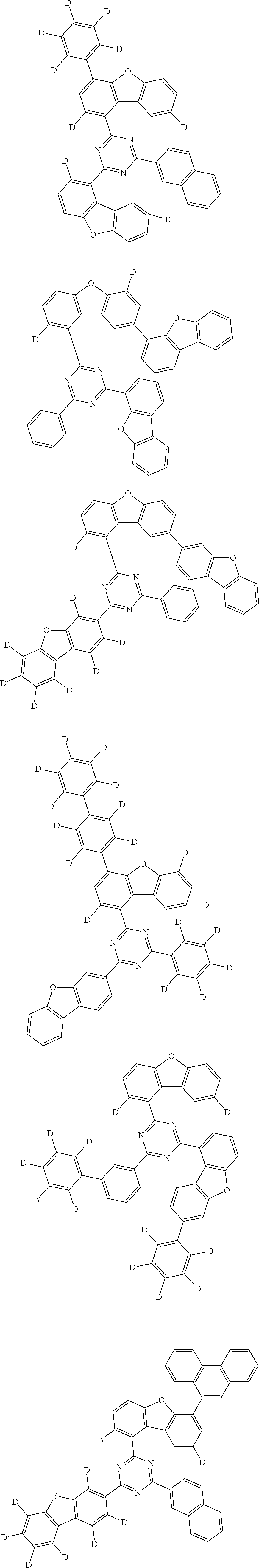

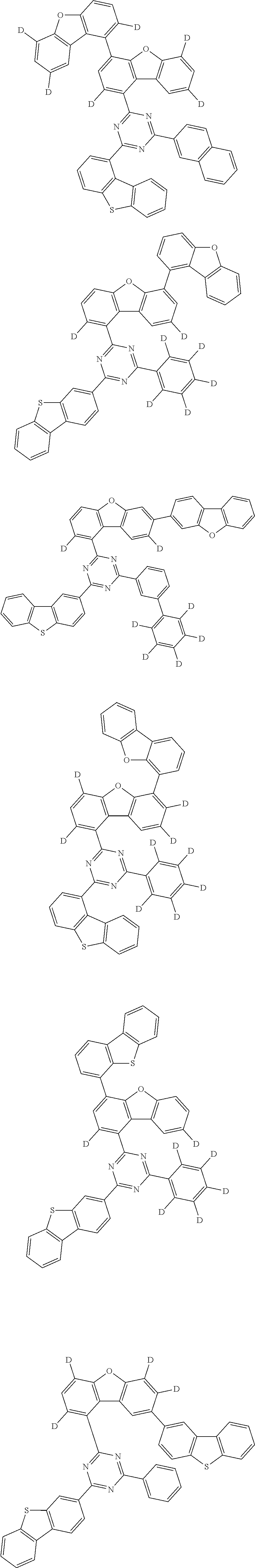

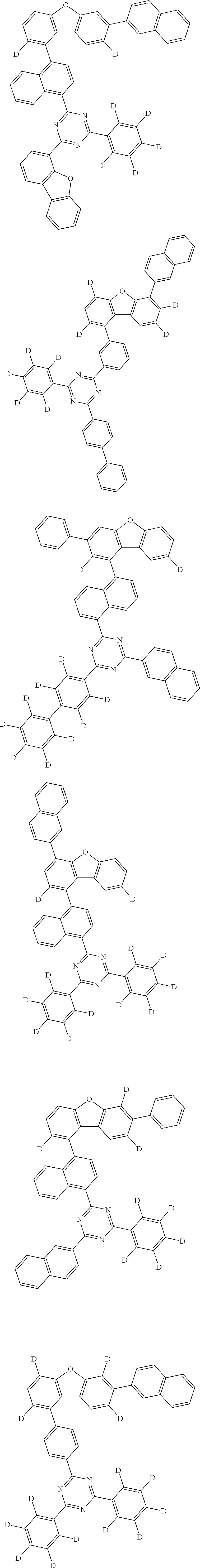

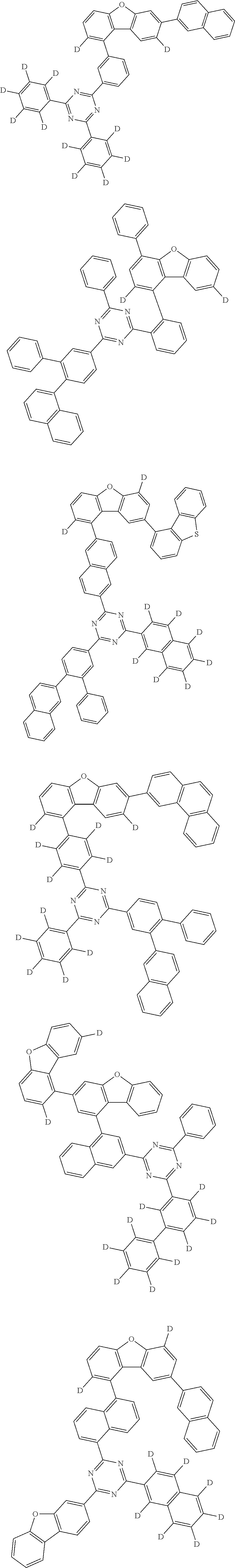

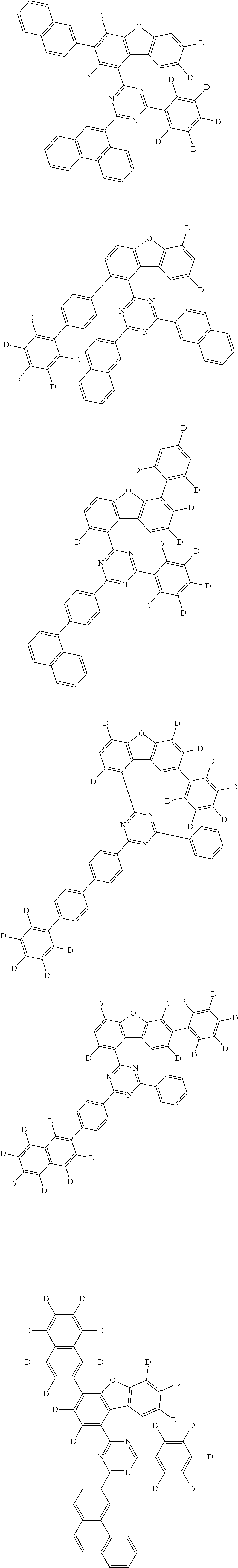

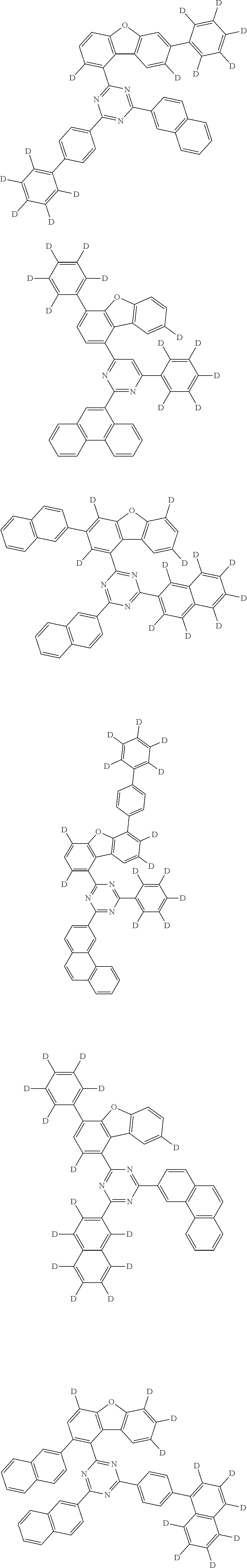

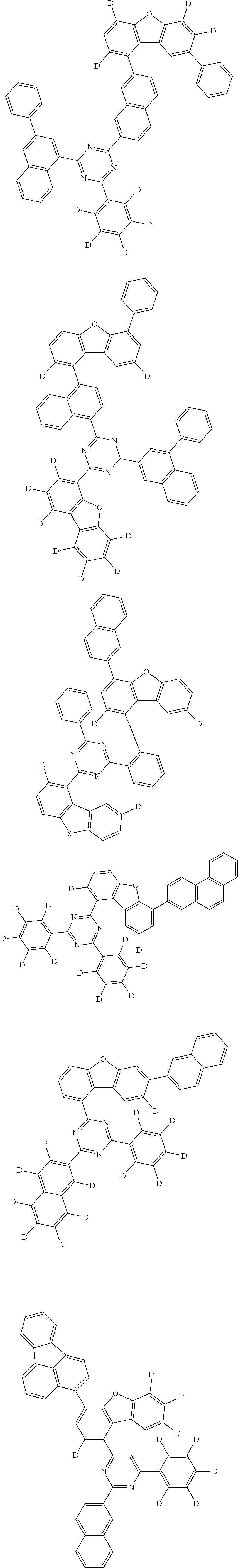

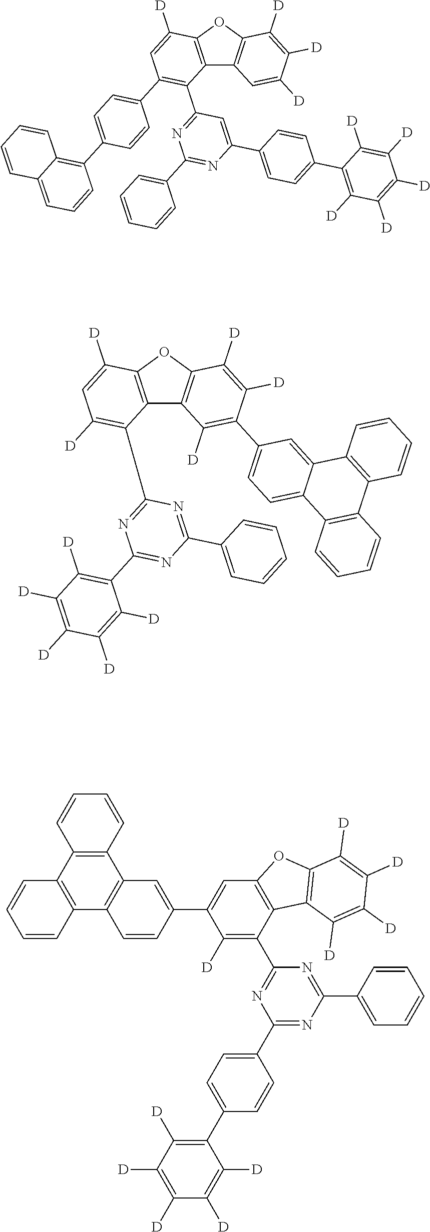 [SMILES: [2H]c1cc2c(oc3c([2H])cc(-c4ccc(-c5cccc6ccccc56)cc4)c(-c4cc(-c5ccc(-c6c([2H])c([2H])c([2H])c([2H])c6[2H])cc5)nc(-c5ccccc5)n4)c32)c([2H])c1[2H] ; [2H]c1cc([2H])c2oc3c([2H])c([2H])c(-c4ccc5c6ccccc6c6ccccc6c5c4)c([2H])c3c2c1-c1nc(-c2ccccc2)nc(-c2c([2H])c([2H])c([2H])c([2H])c2[2H])n1 ; [2H]c1c([2H])c([2H])c(-c2ccc(-c3nc(-c4ccccc4)nc(-c4c([2H])c(-c5ccc6c7ccccc7c7ccccc7c6c5)cc5oc6c([2H])c([2H])c([2H])c([2H])c6c45)n3)cc2)c([2H])c1[2H]]

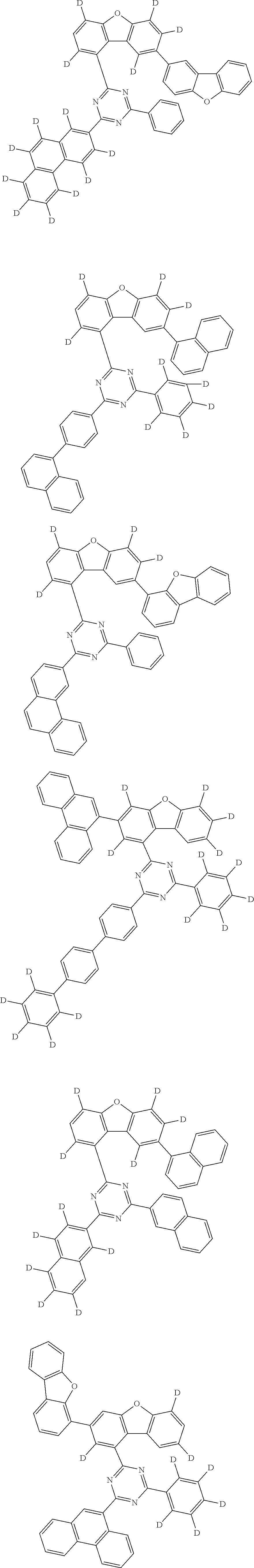

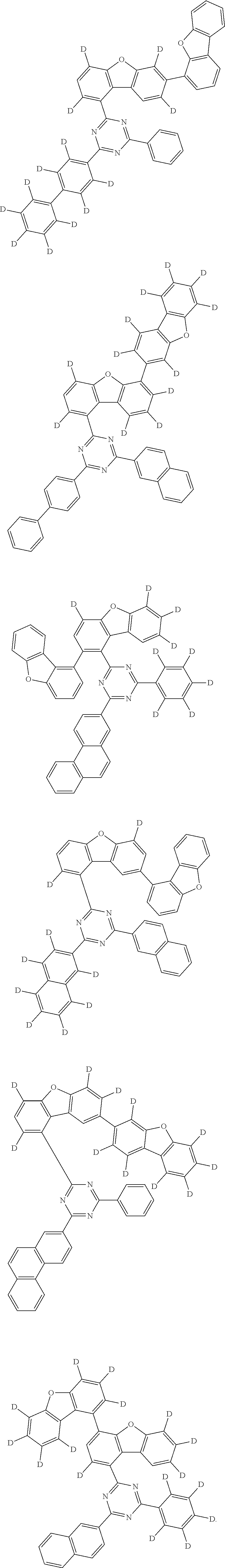

Among the above listed compounds, the compound represented by [structural formula]_{Dn} is a compound of which the corresponding ‘structural formula’ is substituted with n deuteriums.

That is,

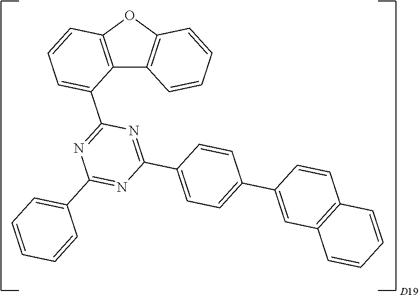

means that

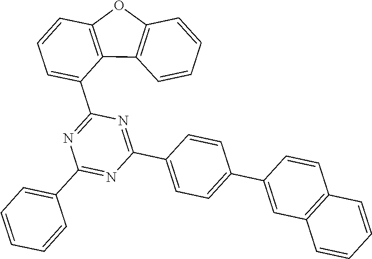 [SMILES: c1ccc(-c2nc(-c3ccc(-c4ccc5ccccc5c4)cc3)nc(-c3cccc4oc5ccccc5c34)n2)cc1]

is substituted with 19 deuteriums.

Further, according to the present disclosure, there is provided a method for preparing the compound represented by Chemical Formula 1.

In one example, the compound represented by Chemical Formula 1 can be prepared by a preparation method as shown in the following Reaction Scheme 1.

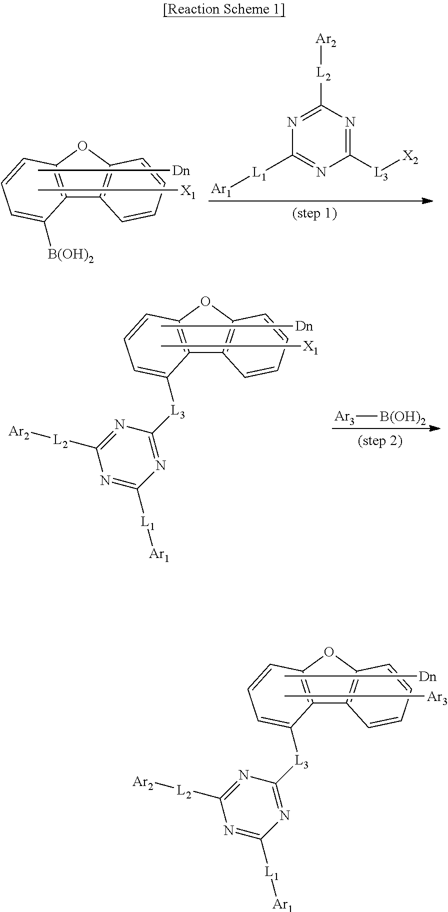

wherein, the definition of the remaining substituents except for X₁ and X₂ are the same as defined in the Chemical Formula 1, and X₁ and X₂ are each independently halogen, preferably chloro or bromo.

The Reaction Scheme 1 is a Suzuki coupling reaction, which is preferably carried out in the presence of a palladium catalyst and a base, and a reactive group for the Suzuki coupling reaction can be changed as known in the art. Further, step 1 and step 2 can be performed by changing the order, if necessary.

Moreover, when the compound represented by Chemical Formula 1 contains deuterium, the target compound can be prepared using a precursor containing deuterium, or alternatively, after preparing a compound containing no deuterium, the target compound can be obtained through a deuterium substitution reaction.

The preparation method can be further embodied in Preparation Examples described hereinafter.

Specifically, Chemical Formula 2 may be represented by any one of the following Chemical Formulas 2-1 to 2-3:

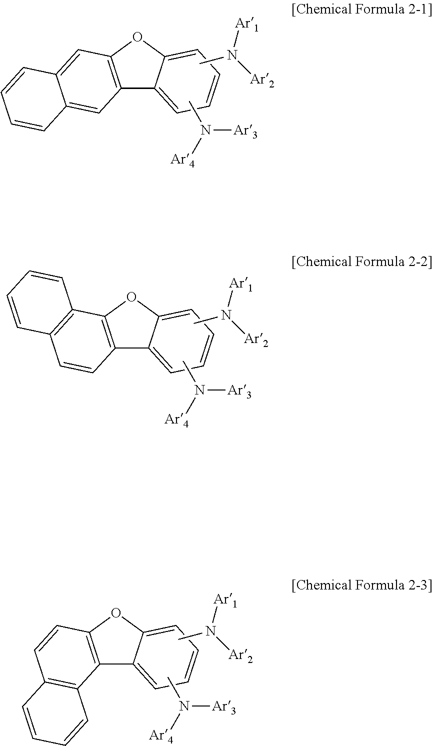

in Chemical Formulas 2-1 to 2-3,
Ar′₁ to Ar′₄ are the same as defined in Chemical Formula 2.

Preferably, Ar′₁ to Ar′₄ are each independently a substituted or unsubstituted C₆₋₂₀ aryl; or a substituted or unsubstituted C₂₋₂₀ heteroaryl containing one or more heteroatoms selected from the group consisting of N, O and S.

More preferably, Ar′₁ to Ar′₄ are each independently phenyl; biphenylyl; terphenylyl; naphthyl; phenylnaphthyl; naphthylphenyl; naphthylbiphenylyl; phenylnaphthylphenyl; phenylterphenylyl; phenanthrenyl; dibenzofuranyl; or dibenzothiophenyl.

Wherein, phenylnaphthyl means naphthyl substituted with one phenyl, naphthylphenyl means phenyl substituted with one naphthyl, naphthylbiphenylyl means biphenylyl substituted with one naphthyl, phenylnaphthylphenyl means phenyl substituted with one phenylnaphthyl, and phenylterphenylyl means terphenylyl substituted with one phenyl.

Representative examples of the compound represented by Chemical Formula 2 are as follows:

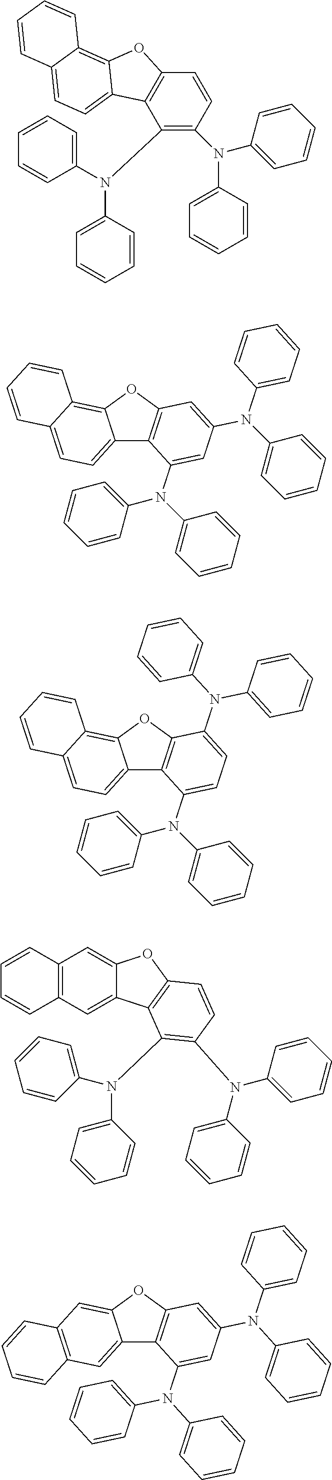

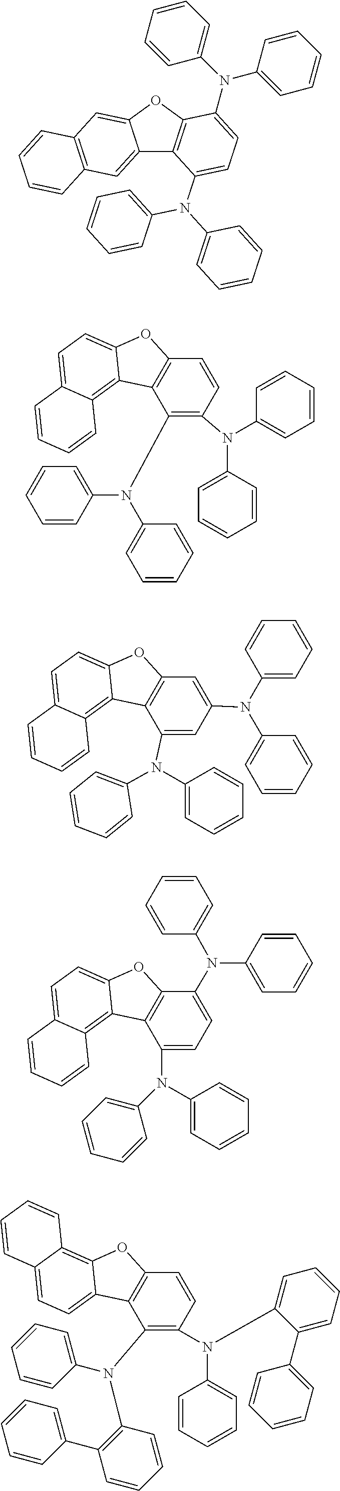

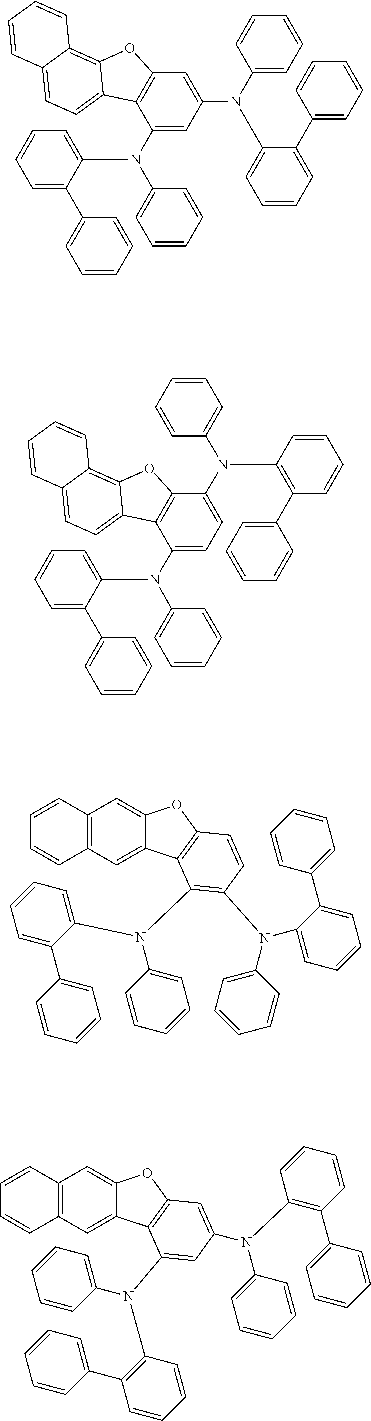 [SMILES: c1ccc(-c2ccccc2N(c2ccccc2)c2cc(N(c3ccccc3)c3ccccc3-c3ccccc3)c3c(c2)oc2c4ccccc4ccc23)cc1 ; c1ccc(-c2ccccc2N(c2ccccc2)c2ccc(N(c3ccccc3)c3ccccc3-c3ccccc3)c3c2oc2c4ccccc4ccc23)cc1 ; c1ccc(-c2ccccc2N(c2ccccc2)c2ccc3oc4cc5ccccc5cc4c3c2N(c2ccccc2)c2ccccc2-c2ccccc2)cc1 ; c1ccc(-c2ccccc2N(c2ccccc2)c2cc(N(c3ccccc3)c3ccccc3-c3ccccc3)c3c(c2)oc2cc4ccccc4cc23)cc1]

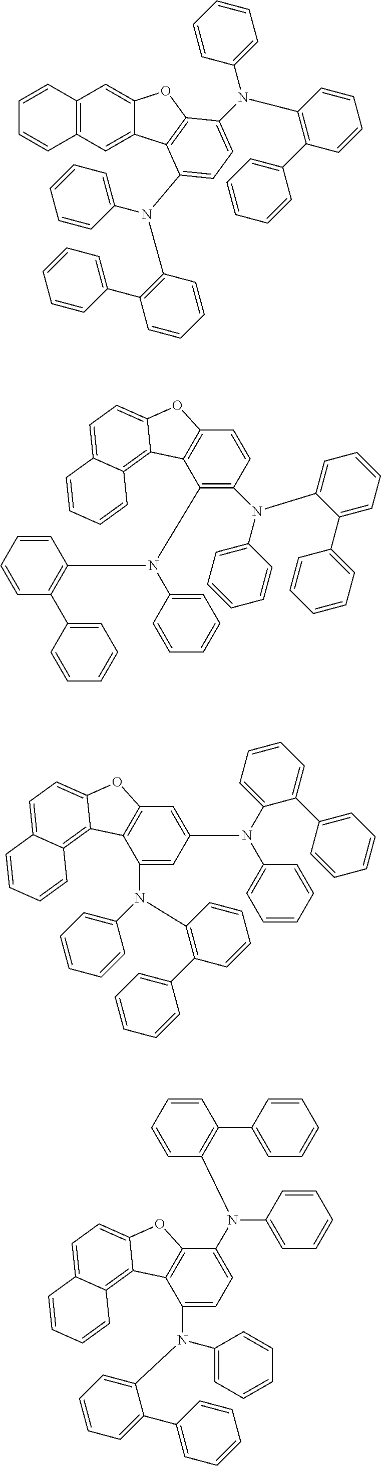 [SMILES: c1ccc(-c2ccccc2N(c2ccccc2)c2ccc(N(c3ccccc3)c3ccccc3-c3ccccc3)c3c2oc2cc4ccccc4cc23)cc1 ; c1ccc(-c2ccccc2N(c2ccccc2)c2ccc3oc4ccc5ccccc5c4c3c2N(c2ccccc2)c2ccccc2-c2ccccc2)cc1 ; c1ccc(-c2ccccc2N(c2ccccc2)c2cc(N(c3ccccc3)c3ccccc3-c3ccccc3)c3c(c2)oc2ccc4ccccc4c23)cc1 ; c1ccc(-c2ccccc2N(c2ccccc2)c2ccc(N(c3ccccc3)c3ccccc3-c3ccccc3)c3c2oc2ccc4ccccc4c23)cc1]

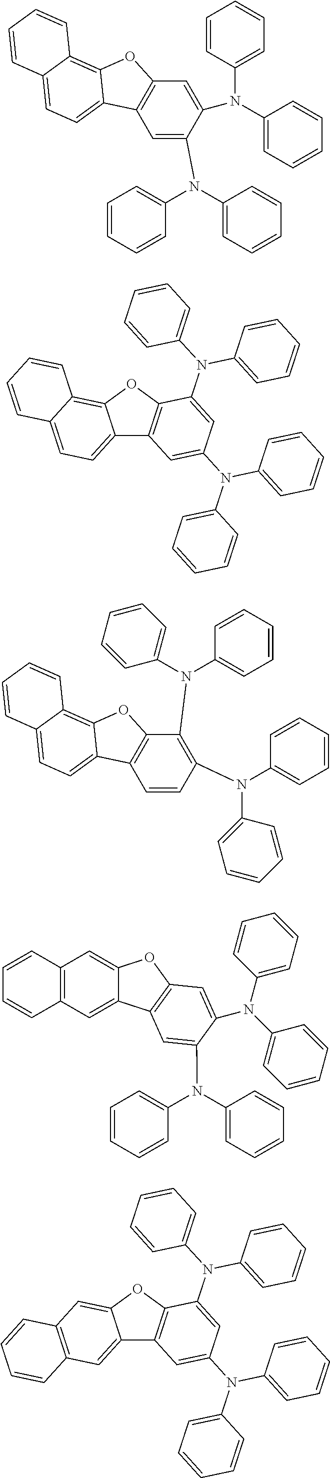

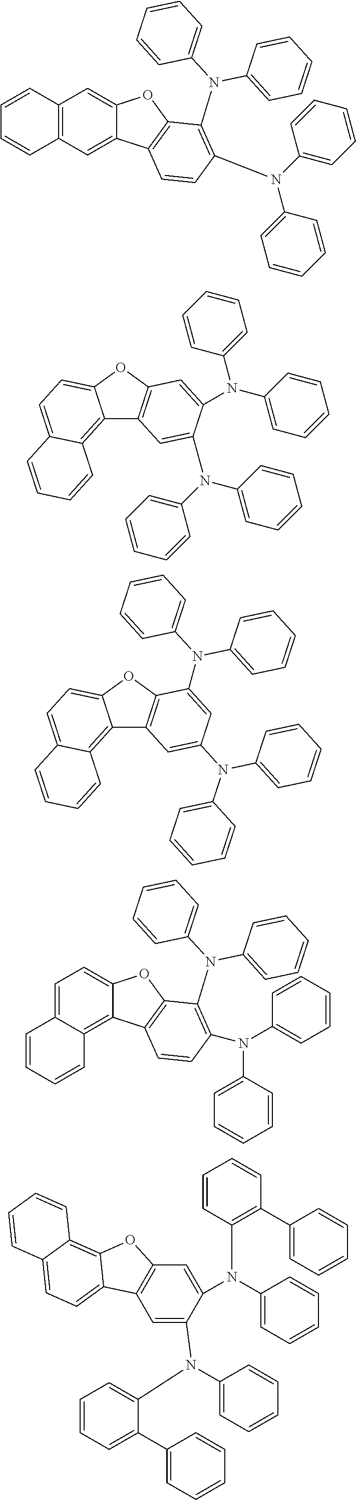

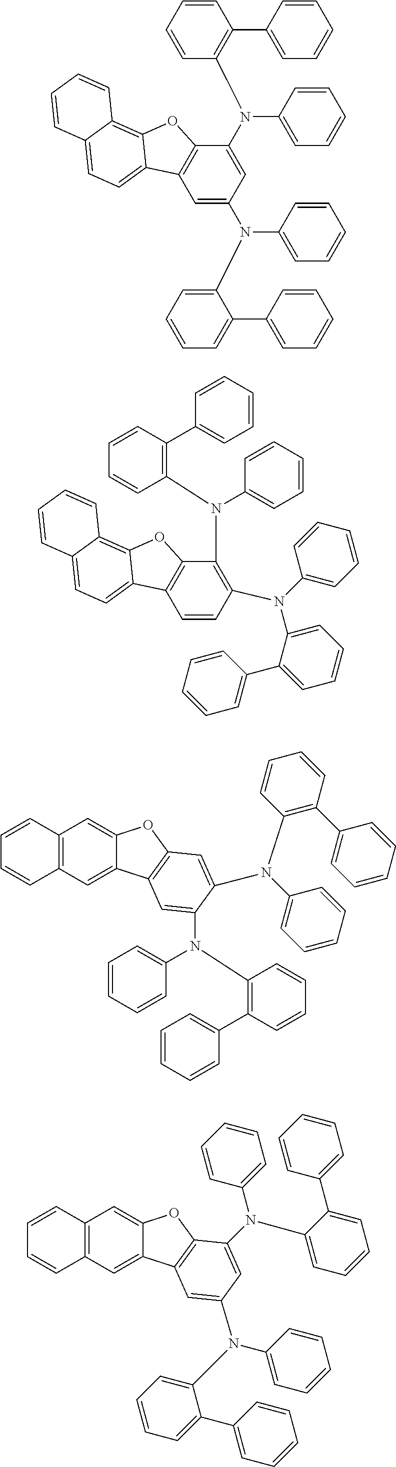

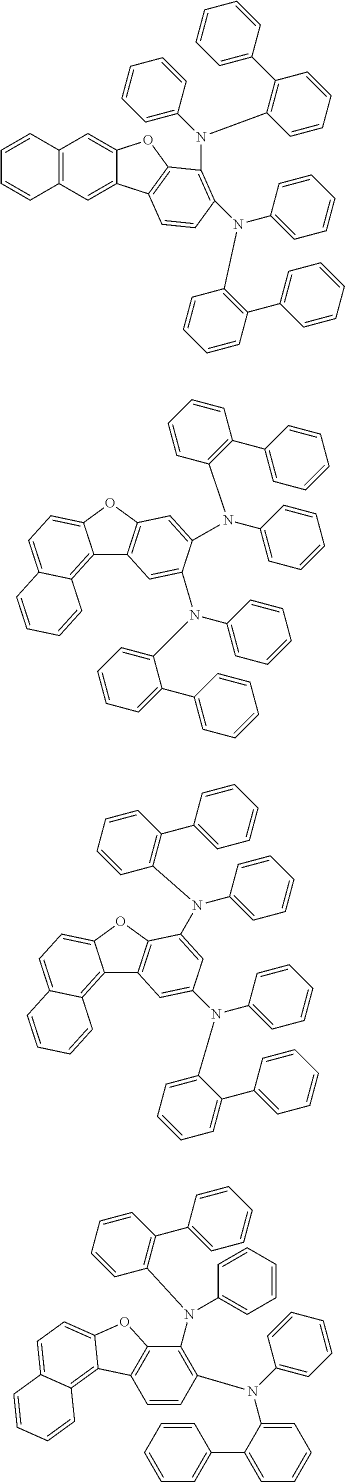

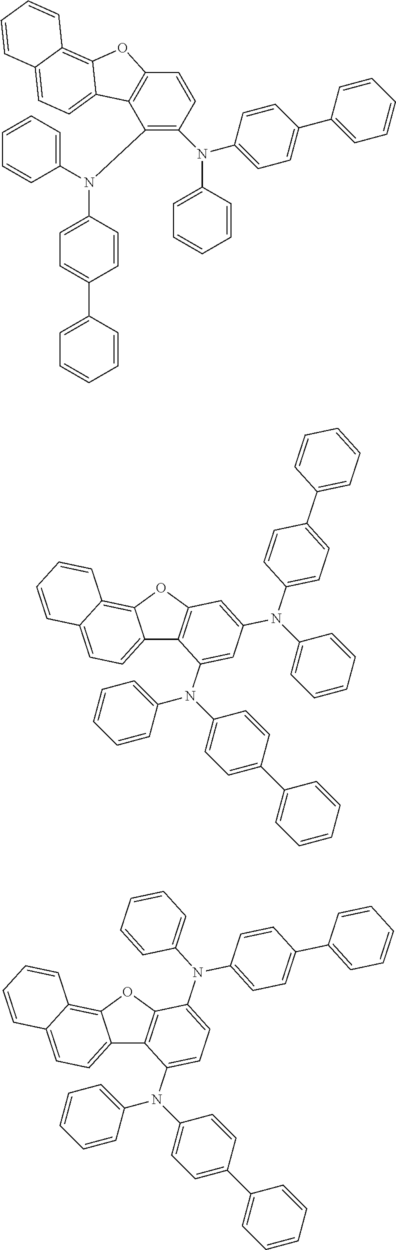 [SMILES: c1ccc(-c2ccc(N(c3ccccc3)c3ccc4oc5c6ccccc6ccc5c4c3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4c(c3)oc3c5ccccc5ccc34)cc2)cc1 ; c1ccc(-c2ccc(N(c3ccccc3)c3ccc(N(c4ccccc4)c4ccc(-c5ccccc5)cc4)c4c3oc3c5ccccc5ccc34)cc2)cc1]

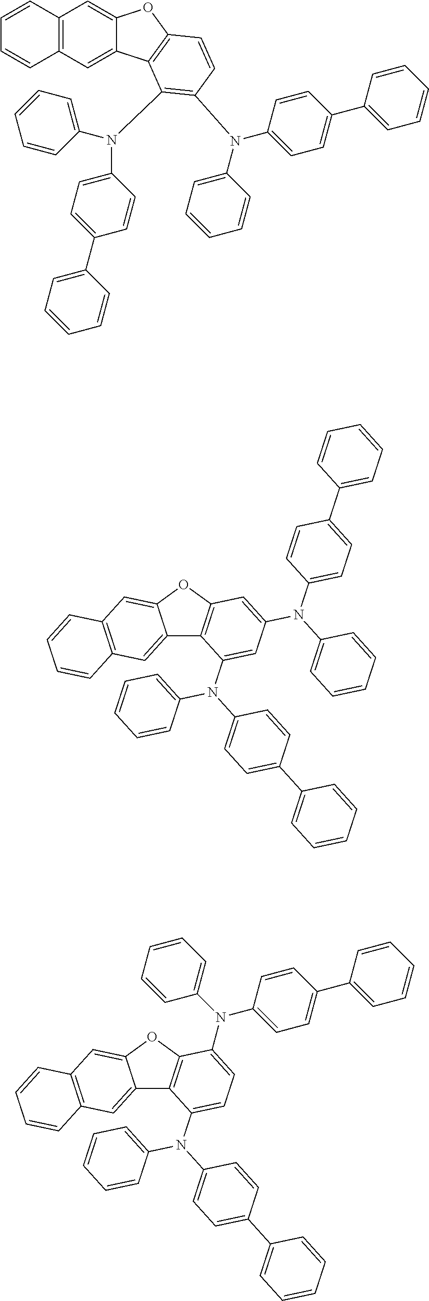 [SMILES: c1ccc(-c2ccc(N(c3ccccc3)c3ccc4oc5cc6ccccc6cc5c4c3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4c(c3)oc3cc5ccccc5cc34)cc2)cc1 ; c1ccc(-c2ccc(N(c3ccccc3)c3ccc(N(c4ccccc4)c4ccc(-c5ccccc5)cc4)c4c3oc3cc5ccccc5cc34)cc2)cc1]

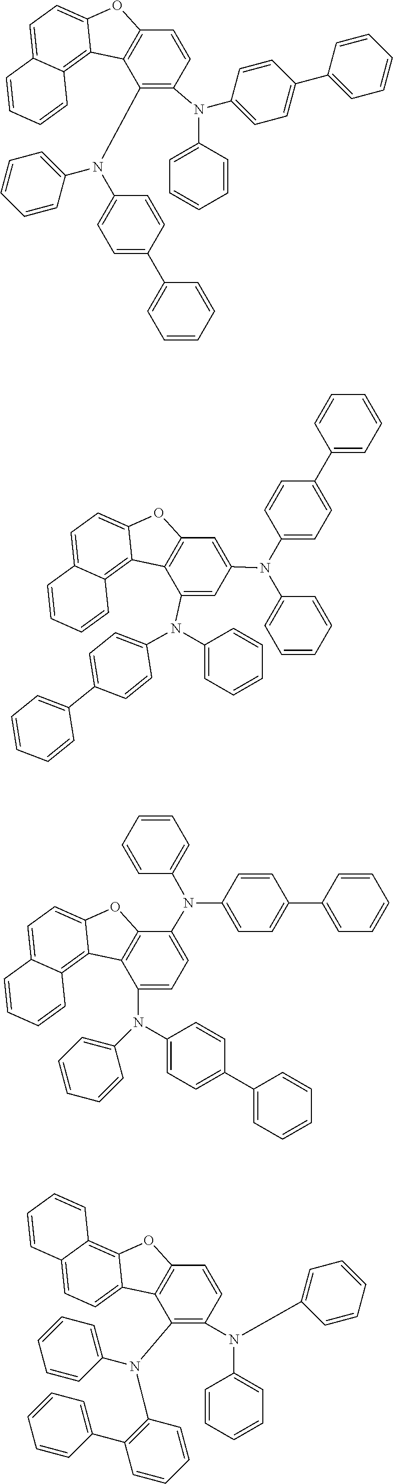 [SMILES: c1ccc(-c2ccc(N(c3ccccc3)c3ccc4oc5ccc6ccccc6c5c4c3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4c(c3)oc3ccc5ccccc5c34)cc2)cc1 ; c1ccc(-c2ccc(N(c3ccccc3)c3ccc(N(c4ccccc4)c4ccc(-c5ccccc5)cc4)c4c3oc3ccc5ccccc5c34)cc2)cc1 ; c1ccc(-c2ccccc2N(c2ccccc2)c2c(N(c3ccccc3)c3ccccc3)ccc3oc4c5ccccc5ccc4c23)cc1]

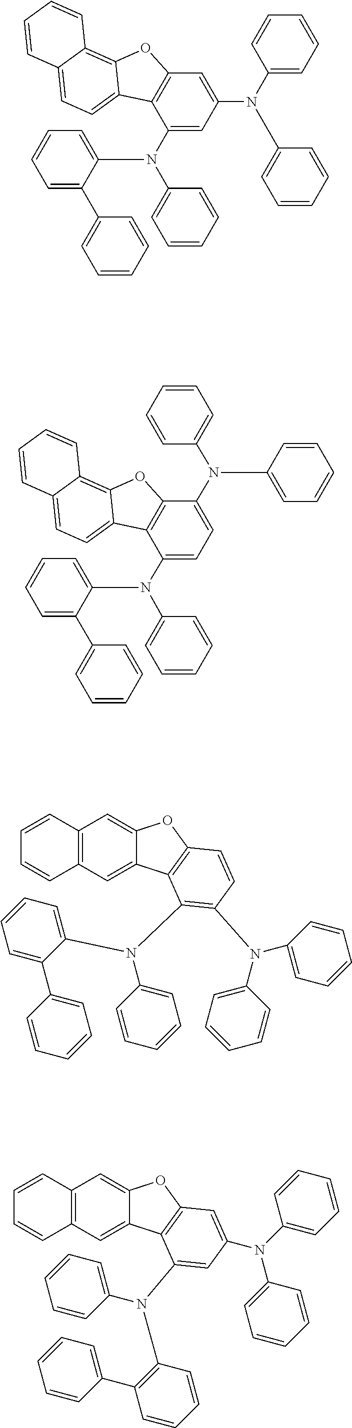 [SMILES: c1ccc(-c2ccccc2N(c2ccccc2)c2cc(N(c3ccccc3)c3ccccc3)cc3oc4c5ccccc5ccc4c23)cc1 ; c1ccc(-c2ccccc2N(c2ccccc2)c2ccc(N(c3ccccc3)c3ccccc3)c3oc4c5ccccc5ccc4c23)cc1 ; c1ccc(-c2ccccc2N(c2ccccc2)c2c(N(c3ccccc3)c3ccccc3)ccc3oc4cc5ccccc5cc4c23)cc1 ; c1ccc(-c2ccccc2N(c2ccccc2)c2cc(N(c3ccccc3)c3ccccc3)cc3oc4cc5ccccc5cc4c23)cc1]

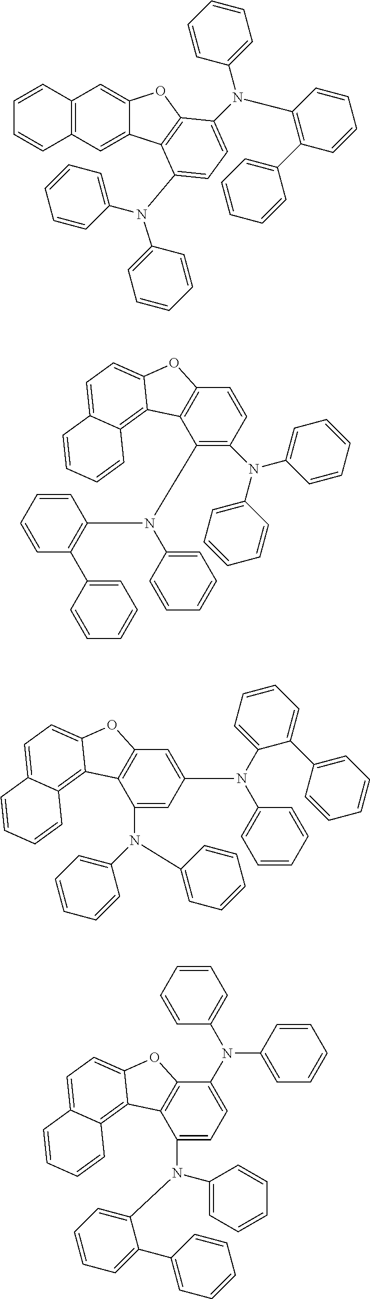 [SMILES: c1ccc(-c2ccccc2N(c2ccccc2)c2ccc(N(c3ccccc3)c3ccccc3)c3c2oc2cc4ccccc4cc23)cc1 ; c1ccc(-c2ccccc2N(c2ccccc2)c2c(N(c3ccccc3)c3ccccc3)ccc3oc4ccc5ccccc5c4c23)cc1 ; c1ccc(-c2ccccc2N(c2ccccc2)c2cc(N(c3ccccc3)c3ccccc3)c3c(c2)oc2ccc4ccccc4c23)cc1 ; c1ccc(-c2ccccc2N(c2ccccc2)c2ccc(N(c3ccccc3)c3ccccc3)c3oc4ccc5ccccc5c4c23)cc1]

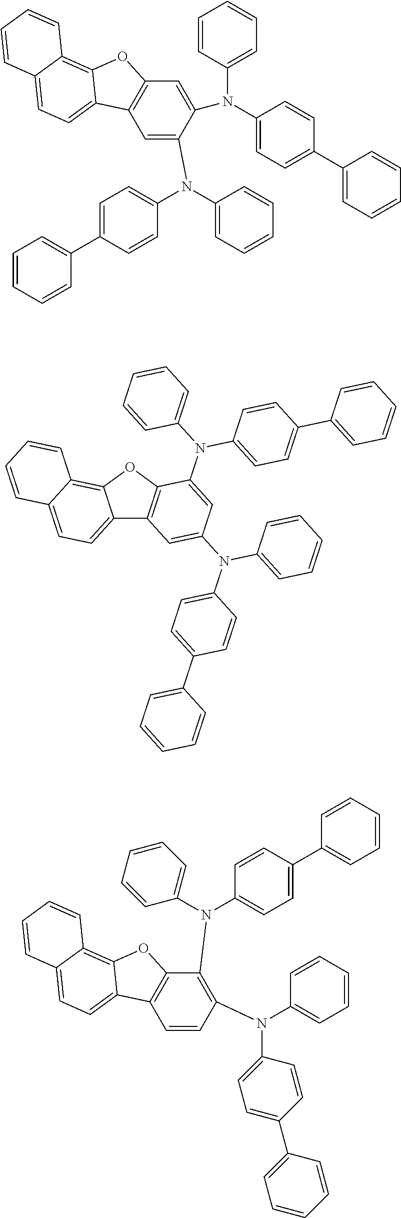 [SMILES: c1ccc(-c2ccc(N(c3ccccc3)c3cc4oc5c6ccccc6ccc5c4cc3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4oc5c6ccccc6ccc5c4c3)cc2)cc1 ; c1ccc(-c2ccc(N(c3ccccc3)c3ccc4c(oc5c6ccccc6ccc45)c3N(c3ccccc3)c3ccc(-c4ccccc4)cc3)cc2)cc1]

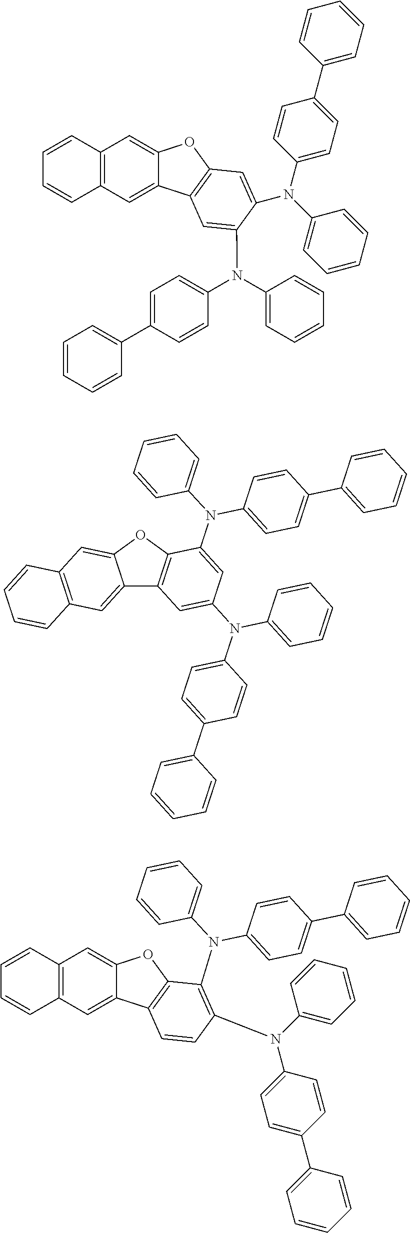

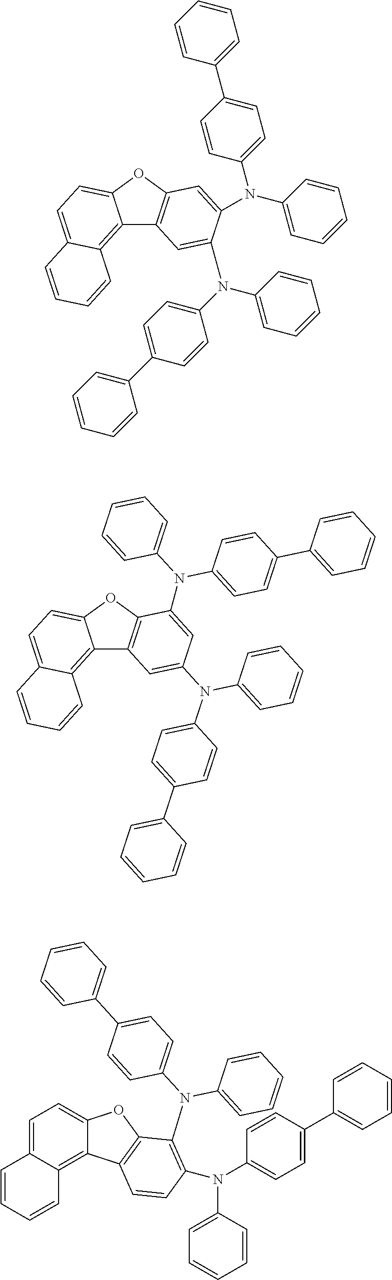 [SMILES: c1ccc(-c2ccc(N(c3ccccc3)c3cc4oc5ccc6ccccc6c5c4cc3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4oc5ccc6ccccc6c5c4c3)cc2)cc1 ; c1ccc(-c2ccc(N(c3ccccc3)c3ccc4c(oc5ccc6ccccc6c54)c3N(c3ccccc3)c3ccc(-c4ccccc4)cc3)cc2)cc1]

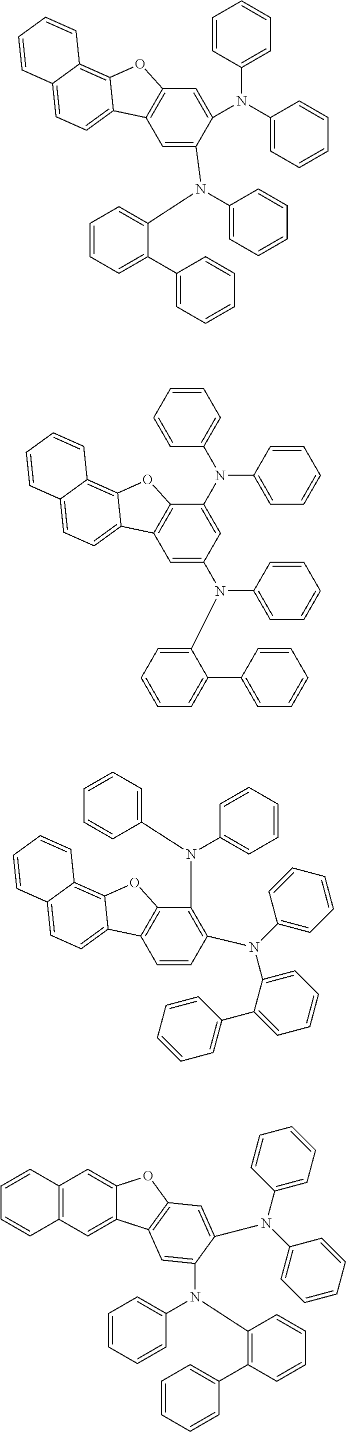 [SMILES: c1ccc(-c2ccccc2N(c2ccccc2)c2cc3c(cc2N(c2ccccc2)c2ccccc2)oc2c4ccccc4ccc32)cc1 ; c1ccc(-c2ccccc2N(c2ccccc2)c2cc(N(c3ccccc3)c3ccccc3)c3oc4c5ccccc5ccc4c3c2)cc1 ; c1ccc(-c2ccccc2N(c2ccccc2)c2ccc3c(oc4c5ccccc5ccc34)c2N(c2ccccc2)c2ccccc2)cc1 ; c1ccc(-c2ccccc2N(c2ccccc2)c2cc3c(cc2N(c2ccccc2)c2ccccc2)oc2cc4ccccc4cc23)cc1]

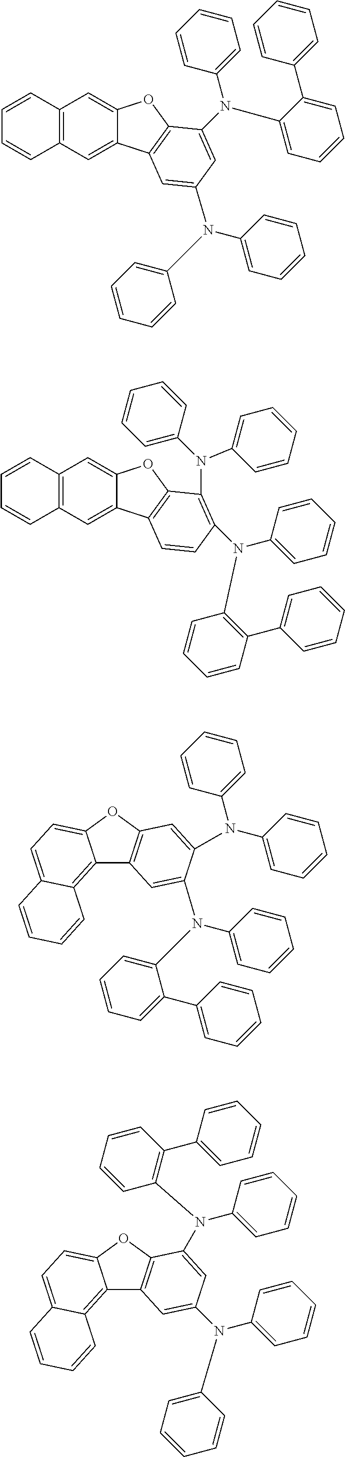 [SMILES: c1ccc(-c2ccccc2N(c2ccccc2)c2cc(N(c3ccccc3)c3ccccc3)cc3c2oc2cc4ccccc4cc23)cc1 ; c1ccc(-c2ccccc2N(c2ccccc2)c2ccc3c(oc4cc5ccccc5cc43)c2N(c2ccccc2)c2ccccc2)cc1 ; c1ccc(-c2ccccc2N(c2ccccc2)c2cc3c(cc2N(c2ccccc2)c2ccccc2)oc2ccc4ccccc4c23)cc1 ; c1ccc(-c2ccccc2N(c2ccccc2)c2cc(N(c3ccccc3)c3ccccc3)cc3c2oc2ccc4ccccc4c23)cc1]

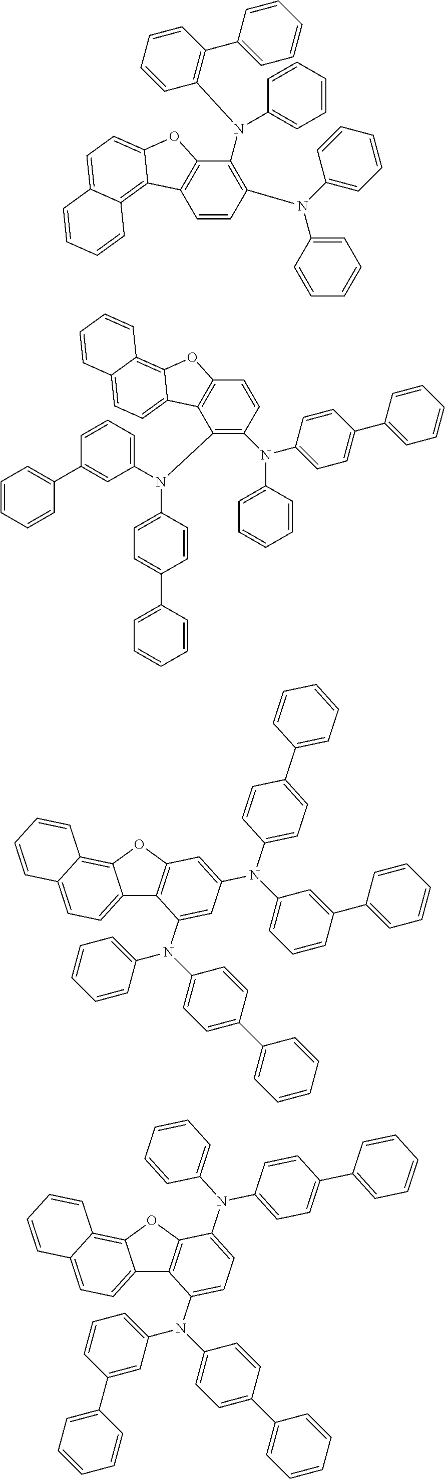 [SMILES: c1ccc(-c2ccccc2N(c2ccccc2)c2c(N(c3ccccc3)c3ccccc3)ccc3c2oc2ccc4ccccc4c23)cc1 ; c1ccc(-c2ccc(N(c3ccccc3)c3ccc4oc5c6ccccc6ccc5c4c3N(c3ccc(-c4ccccc4)cc3)c3cccc(-c4ccccc4)c3)cc2)cc1 ; c1ccc(-c2ccc(N(c3cccc(-c4ccccc4)c3)c3cc(N(c4ccccc4)c4ccc(-c5ccccc5)cc4)c4c(c3)oc3c5ccccc5ccc34)cc2)cc1 ; c1ccc(-c2ccc(N(c3ccccc3)c3ccc(N(c4ccc(-c5ccccc5)cc4)c4cccc(-c5ccccc5)c4)c4c3oc3c5ccccc5ccc34)cc2)cc1]

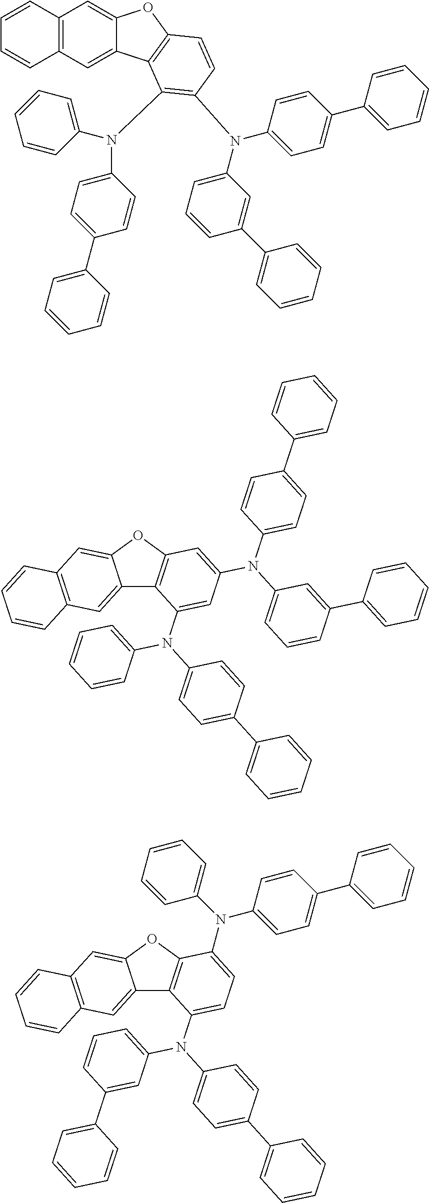 [SMILES: c1ccc(-c2ccc(N(c3cccc(-c4ccccc4)c3)c3ccc4oc5cc6ccccc6cc5c4c3N(c3ccccc3)c3ccc(-c4ccccc4)cc3)cc2)cc1 ; c1ccc(-c2ccc(N(c3cccc(-c4ccccc4)c3)c3cc(N(c4ccccc4)c4ccc(-c5ccccc5)cc4)c4c(c3)oc3cc5ccccc5cc34)cc2)cc1 ; c1ccc(-c2ccc(N(c3ccccc3)c3ccc(N(c4ccc(-c5ccccc5)cc4)c4cccc(-c5ccccc5)c4)c4c3oc3cc5ccccc5cc34)cc2)cc1]

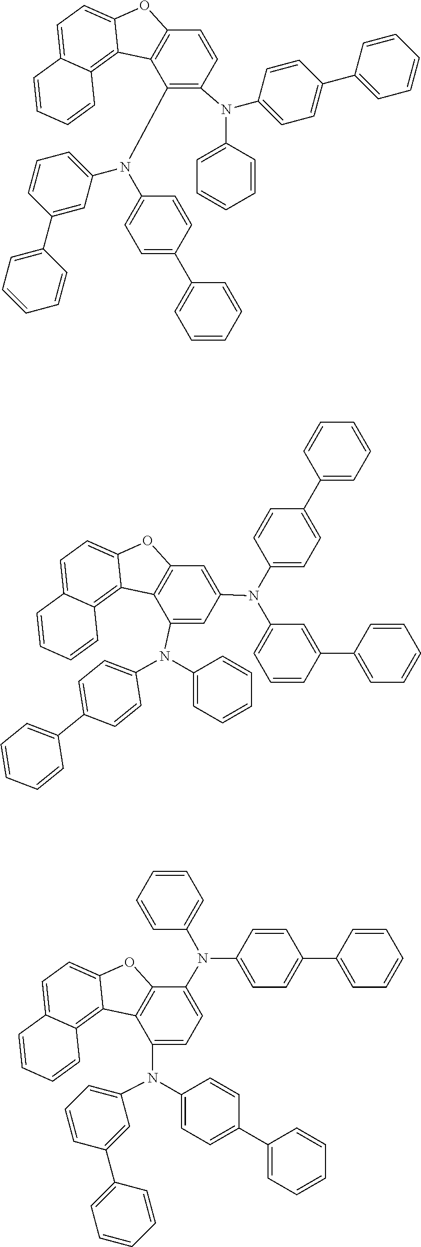 [SMILES: c1ccc(-c2ccc(N(c3ccccc3)c3ccc4oc5ccc6ccccc6c5c4c3N(c3ccc(-c4ccccc4)cc3)c3cccc(-c4ccccc4)c3)cc2)cc1 ; c1ccc(-c2ccc(N(c3cccc(-c4ccccc4)c3)c3cc(N(c4ccccc4)c4ccc(-c5ccccc5)cc4)c4c(c3)oc3ccc5ccccc5c34)cc2)cc1 ; c1ccc(-c2ccc(N(c3ccccc3)c3ccc(N(c4ccc(-c5ccccc5)cc4)c4cccc(-c5ccccc5)c4)c4c3oc3ccc5ccccc5c34)cc2)cc1]

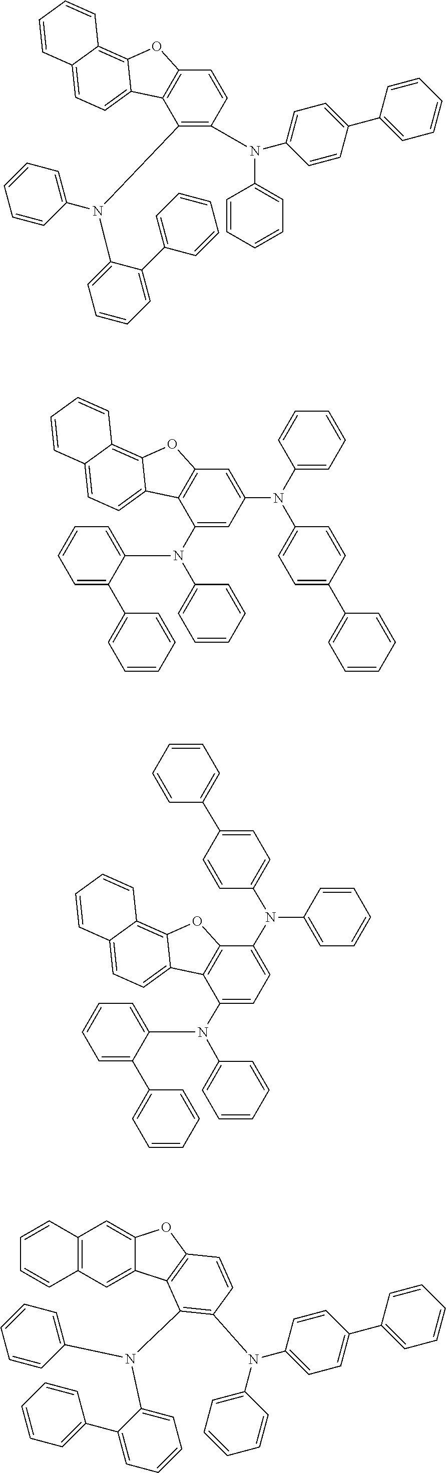 [SMILES: c1ccc(-c2ccc(N(c3ccccc3)c3ccc4oc5c6ccccc6ccc5c4c3N(c3ccccc3)c3ccccc3-c3ccccc3)cc2)cc1 ; c1ccc(-c2ccc(N(c3ccccc3)c3cc(N(c4ccccc4)c4ccccc4-c4ccccc4)c4c(c3)oc3c5ccccc5ccc34)cc2)cc1 ; c1ccc(-c2ccc(N(c3ccccc3)c3ccc(N(c4ccccc4)c4ccccc4-c4ccccc4)c4c3oc3c5ccccc5ccc34)cc2)cc1 ; c1ccc(-c2ccc(N(c3ccccc3)c3ccc4oc5cc6ccccc6cc5c4c3N(c3ccccc3)c3ccccc3-c3ccccc3)cc2)cc1]

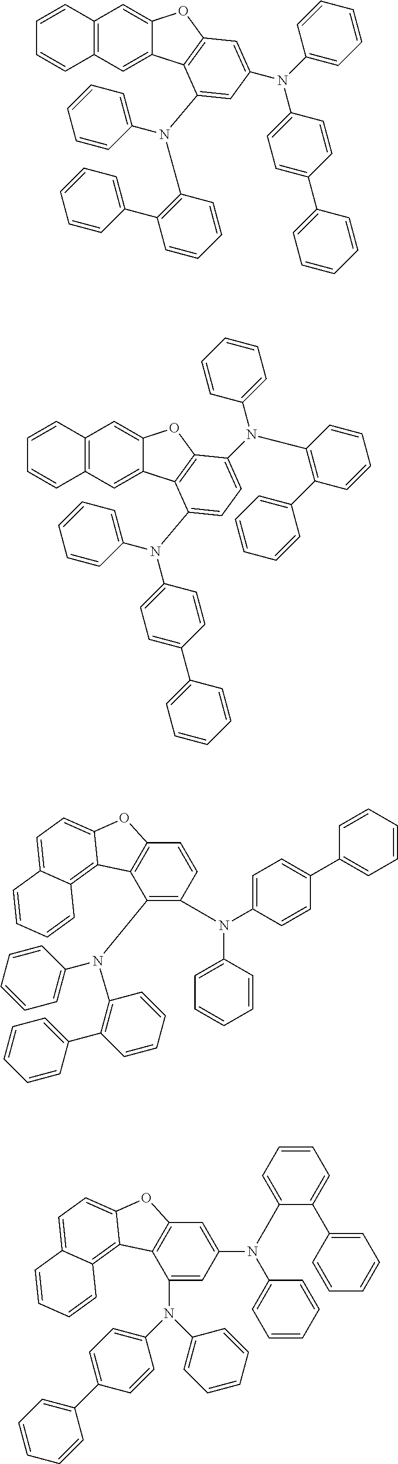 [SMILES: c1ccc(-c2ccc(N(c3ccccc3)c3cc(N(c4ccccc4)c4ccccc4-c4ccccc4)c4c(c3)oc3cc5ccccc5cc34)cc2)cc1 ; c1ccc(-c2ccc(N(c3ccccc3)c3ccc(N(c4ccccc4)c4ccccc4-c4ccccc4)c4oc5cc6ccccc6cc5c34)cc2)cc1 ; c1ccc(-c2ccc(N(c3ccccc3)c3ccc4oc5ccc6ccccc6c5c4c3N(c3ccccc3)c3ccccc3-c3ccccc3)cc2)cc1 ; c1ccc(-c2ccc(N(c3ccccc3)c3cc(N(c4ccccc4)c4ccccc4-c4ccccc4)cc4oc5ccc6ccccc6c5c34)cc2)cc1]

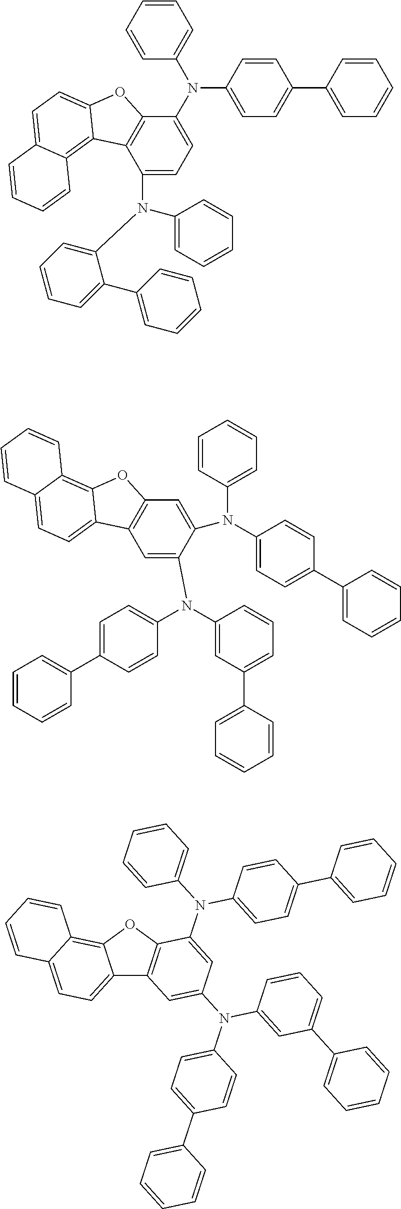 [SMILES: c1ccc(-c2ccc(N(c3ccccc3)c3ccc(N(c4ccccc4)c4ccccc4-c4ccccc4)c4c3oc3ccc5ccccc5c34)cc2)cc1 ; c1ccc(-c2ccc(N(c3ccccc3)c3cc4oc5c6ccccc6ccc5c4cc3N(c3ccc(-c4ccccc4)cc3)c3cccc(-c4ccccc4)c3)cc2)cc1 ; c1ccc(-c2ccc(N(c3cccc(-c4ccccc4)c3)c3cc(N(c4ccccc4)c4ccc(-c5ccccc5)cc4)c4oc5c6ccccc6ccc5c4c3)cc2)cc1]

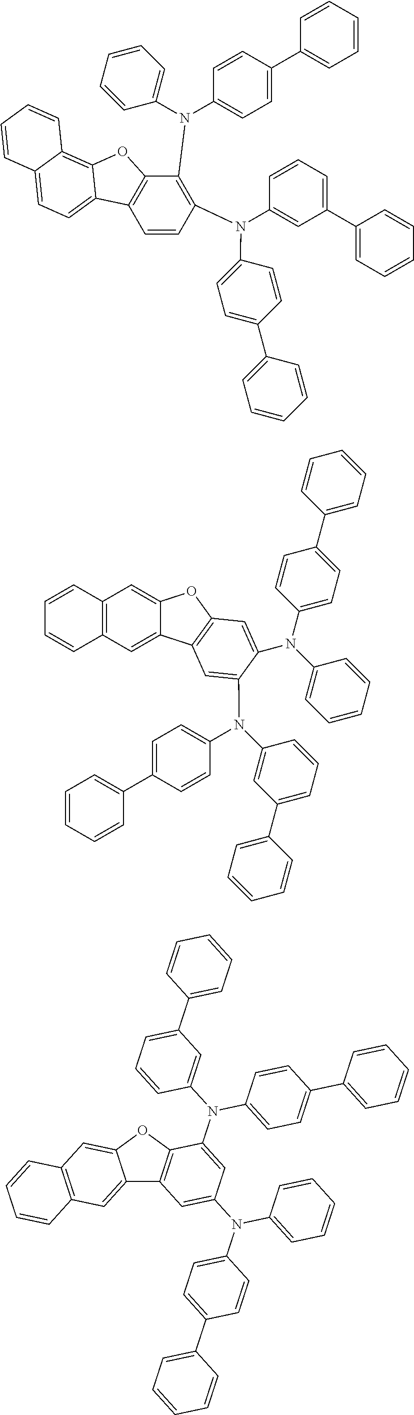

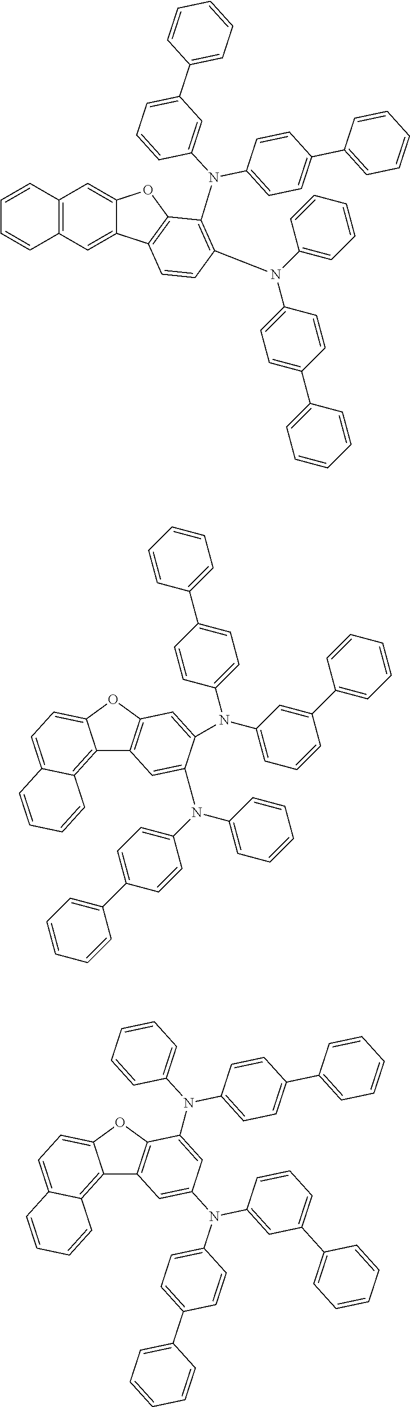 [SMILES: c1ccc(-c2ccc(N(c3ccccc3)c3ccc4c(oc5cc6ccccc6cc54)c3N(c3ccc(-c4ccccc4)cc3)c3cccc(-c4ccccc4)c3)cc2)cc1 ; c1ccc(-c2ccc(N(c3cccc(-c4ccccc4)c3)c3cc4oc5ccc6ccccc6c5c4cc3N(c3ccccc3)c3ccc(-c4ccccc4)cc3)cc2)cc1 ; c1ccc(-c2ccc(N(c3cccc(-c4ccccc4)c3)c3cc(N(c4ccccc4)c4ccc(-c5ccccc5)cc4)c4oc5ccc6ccccc6c5c4c3)cc2)cc1]

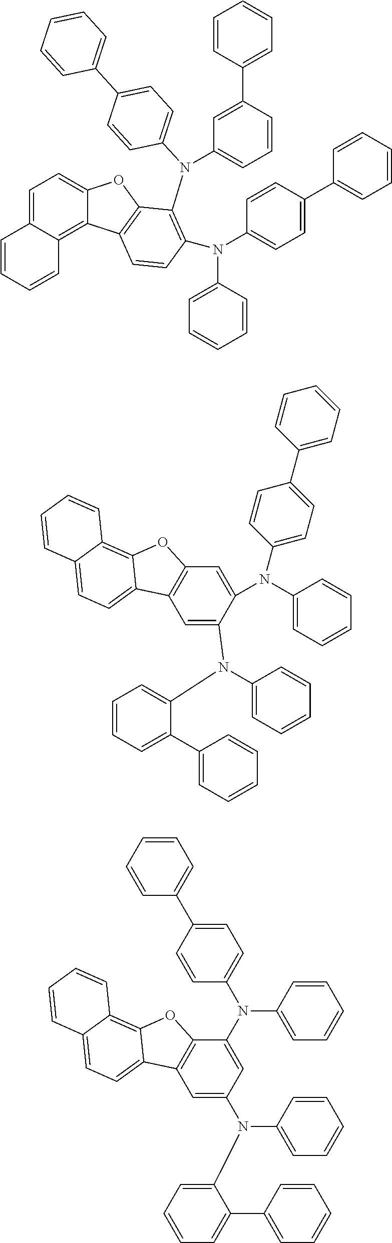 [SMILES: c1ccc(-c2ccc(N(c3ccccc3)c3ccc4c(oc5ccc6ccccc6c54)c3N(c3ccc(-c4ccccc4)cc3)c3cccc(-c4ccccc4)c3)cc2)cc1 ; c1ccc(-c2ccc(N(c3ccccc3)c3cc4oc5c6ccccc6ccc5c4cc3N(c3ccccc3)c3ccccc3-c3ccccc3)cc2)cc1 ; c1ccc(-c2ccc(N(c3ccccc3)c3cc(N(c4ccccc4)c4ccccc4-c4ccccc4)cc4c3oc3c5ccccc5ccc43)cc2)cc1]

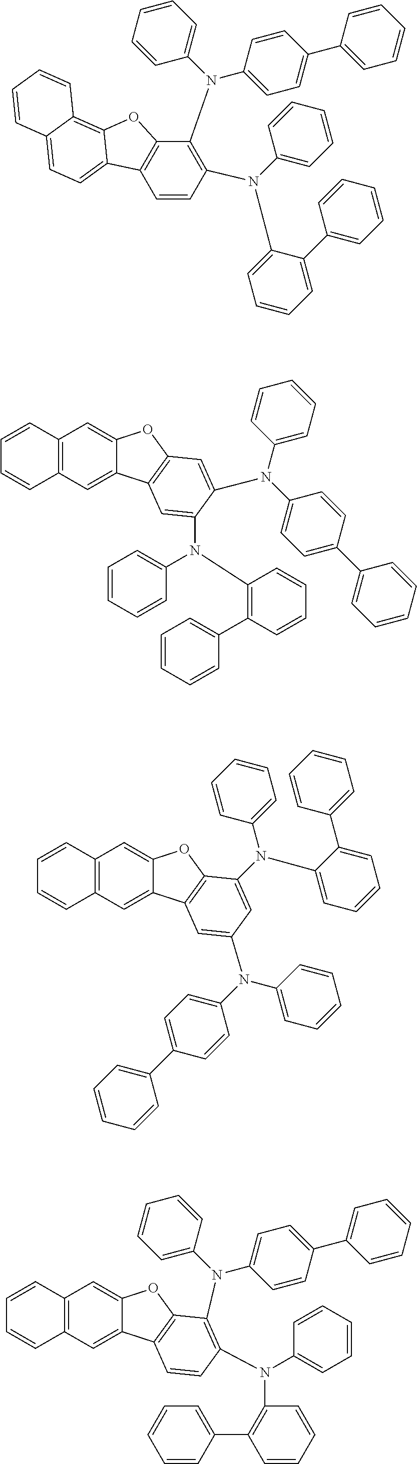 [SMILES: c1ccc(-c2ccc(N(c3ccccc3)c3c(N(c4ccccc4)c4ccccc4-c4ccccc4)ccc4c3oc3c5ccccc5ccc43)cc2)cc1 ; c1ccc(-c2ccc(N(c3ccccc3)c3cc4oc5cc6ccccc6cc5c4cc3N(c3ccccc3)c3ccccc3-c3ccccc3)cc2)cc1 ; c1ccc(-c2ccc(N(c3ccccc3)c3cc(N(c4ccccc4)c4ccccc4-c4ccccc4)c4oc5cc6ccccc6cc5c4c3)cc2)cc1 ; c1ccc(-c2ccc(N(c3ccccc3)c3c(N(c4ccccc4)c4ccccc4-c4ccccc4)ccc4c3oc3cc5ccccc5cc34)cc2)cc1]

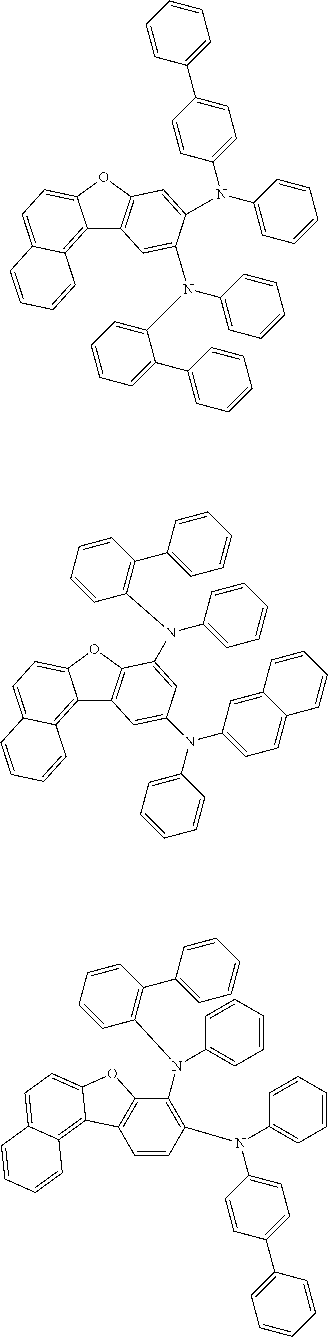 [SMILES: c1ccc(-c2ccc(N(c3ccccc3)c3cc4oc5ccc6ccccc6c5c4cc3N(c3ccccc3)c3ccccc3-c3ccccc3)cc2)cc1 ; c1ccc(-c2ccccc2N(c2ccccc2)c2cc(N(c3ccccc3)c3ccc4ccccc4c3)cc3c2oc2ccc4ccccc4c23)cc1 ; c1ccc(-c2ccc(N(c3ccccc3)c3ccc4c(oc5ccc6ccccc6c54)c3N(c3ccccc3)c3ccccc3-c3ccccc3)cc2)cc1]

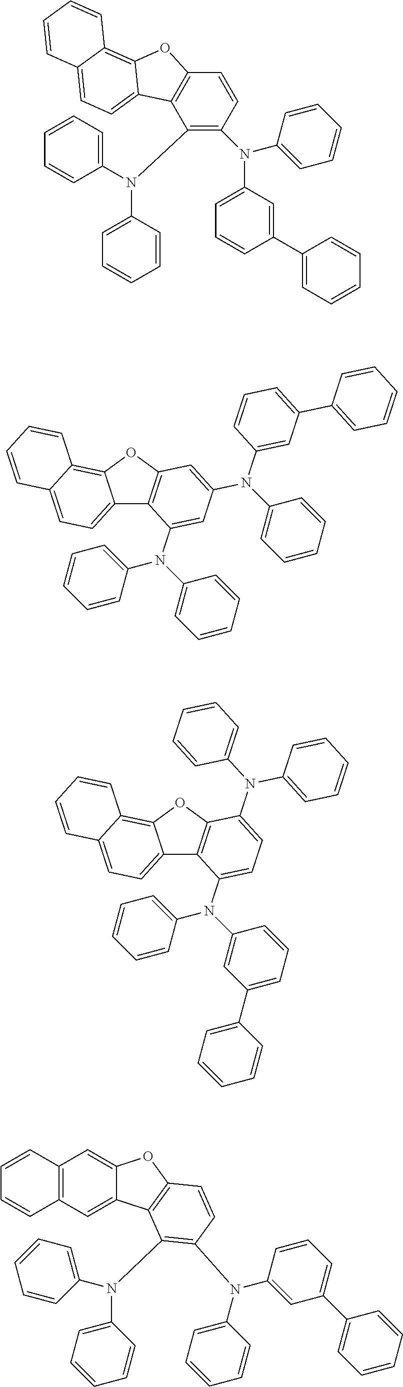 [SMILES: c1ccc(-c2cccc(N(c3ccccc3)c3ccc4oc5c6ccccc6ccc5c4c3N(c3ccccc3)c3ccccc3)c2)cc1 ; c1ccc(-c2cccc(N(c3ccccc3)c3cc(N(c4ccccc4)c4ccccc4)c4c(c3)oc3c5ccccc5ccc34)c2)cc1 ; c1ccc(-c2cccc(N(c3ccccc3)c3ccc(N(c4ccccc4)c4ccccc4)c4oc5c6ccccc6ccc5c34)c2)cc1 ; c1ccc(-c2cccc(N(c3ccccc3)c3ccc4oc5cc6ccccc6cc5c4c3N(c3ccccc3)c3ccccc3)c2)cc1]

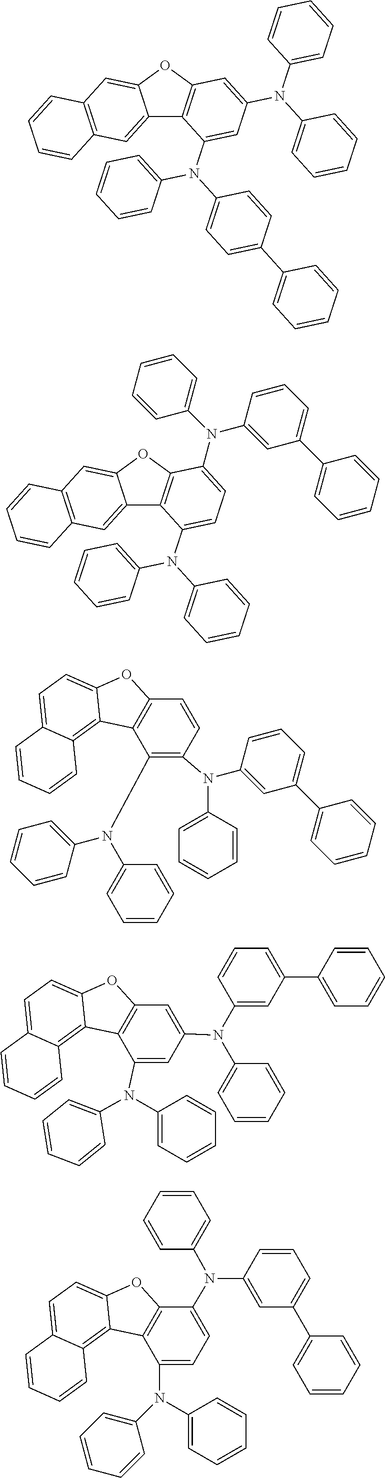

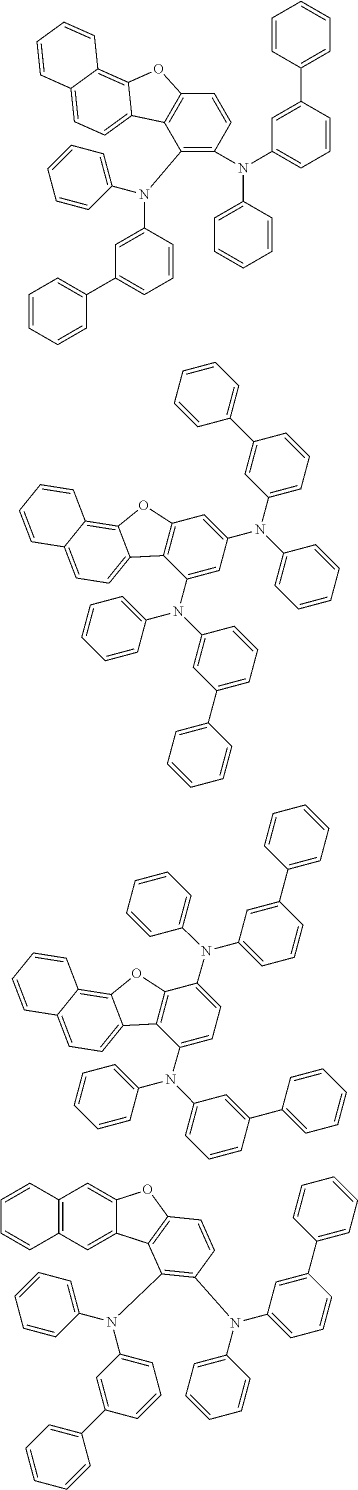 [SMILES: c1ccc(-c2cccc(N(c3ccccc3)c3ccc4oc5c6ccccc6ccc5c4c3N(c3ccccc3)c3cccc(-c4ccccc4)c3)c2)cc1 ; c1ccc(-c2cccc(N(c3ccccc3)c3cc(N(c4ccccc4)c4cccc(-c5ccccc5)c4)c4c(c3)oc3c5ccccc5ccc34)c2)cc1 ; c1ccc(-c2cccc(N(c3ccccc3)c3ccc(N(c4ccccc4)c4cccc(-c5ccccc5)c4)c4c3oc3c5ccccc5ccc34)c2)cc1 ; c1ccc(-c2cccc(N(c3ccccc3)c3ccc4oc5cc6ccccc6cc5c4c3N(c3ccccc3)c3cccc(-c4ccccc4)c3)c2)cc1]

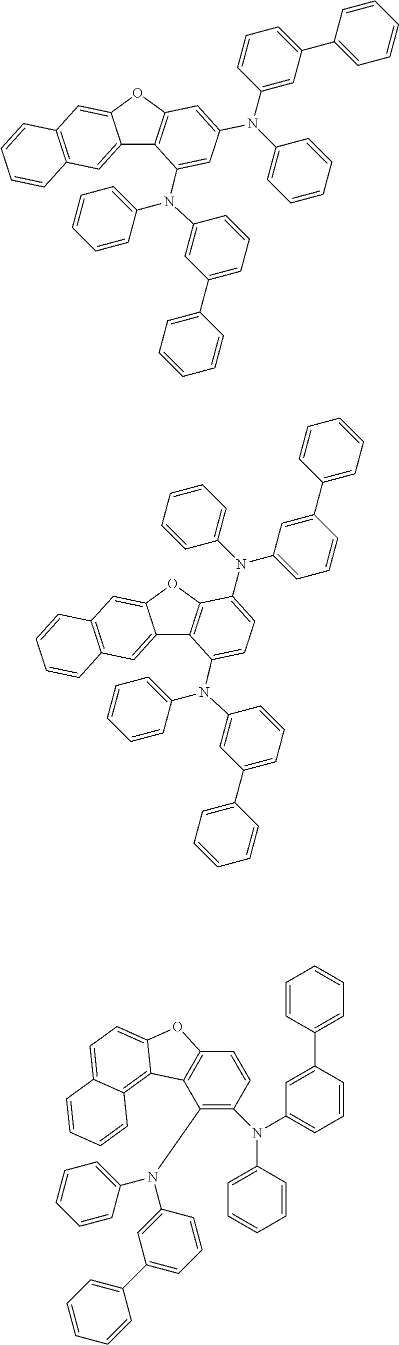 [SMILES: c1ccc(-c2cccc(N(c3ccccc3)c3cc(N(c4ccccc4)c4cccc(-c5ccccc5)c4)c4c(c3)oc3cc5ccccc5cc34)c2)cc1 ; c1ccc(-c2cccc(N(c3ccccc3)c3ccc(N(c4ccccc4)c4cccc(-c5ccccc5)c4)c4c3oc3cc5ccccc5cc34)c2)cc1 ; c1ccc(-c2cccc(N(c3ccccc3)c3ccc4oc5ccc6ccccc6c5c4c3N(c3ccccc3)c3cccc(-c4ccccc4)c3)c2)cc1]

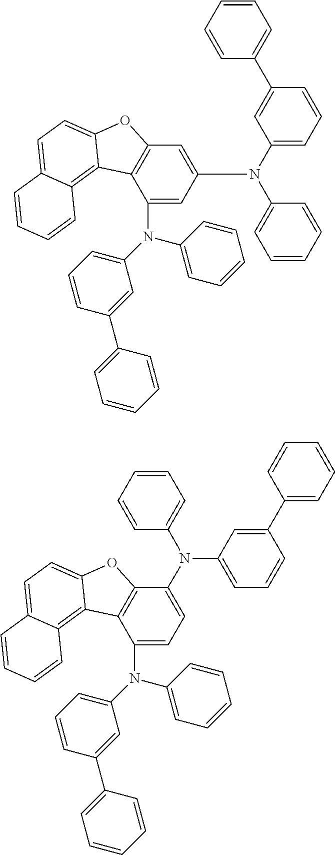 [SMILES: c1ccc(-c2cccc(N(c3ccccc3)c3cc(N(c4ccccc4)c4cccc(-c5ccccc5)c4)c4c(c3)oc3ccc5ccccc5c34)c2)cc1 ; c1ccc(-c2cccc(N(c3ccccc3)c3ccc(N(c4ccccc4)c4cccc(-c5ccccc5)c4)c4c3oc3ccc5ccccc5c34)c2)cc1]

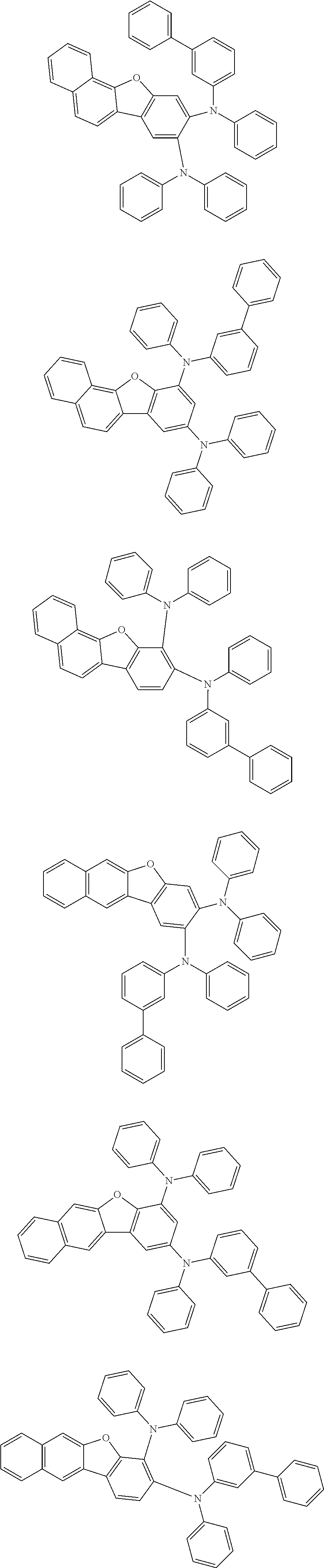 [SMILES: c1ccc(-c2cccc(N(c3ccccc3)c3cc4oc5c6ccccc6ccc5c4cc3N(c3ccccc3)c3ccccc3)c2)cc1 ; c1ccc(-c2cccc(N(c3ccccc3)c3cc(N(c4ccccc4)c4ccccc4)cc4c3oc3c5ccccc5ccc43)c2)cc1 ; c1ccc(-c2cccc(N(c3ccccc3)c3ccc4c(oc5c6ccccc6ccc45)c3N(c3ccccc3)c3ccccc3)c2)cc1 ; c1ccc(-c2cccc(N(c3ccccc3)c3cc4c(cc3N(c3ccccc3)c3ccccc3)oc3cc5ccccc5cc34)c2)cc1 ; c1ccc(-c2cccc(N(c3ccccc3)c3cc(N(c4ccccc4)c4ccccc4)c4oc5cc6ccccc6cc5c4c3)c2)cc1 ; c1ccc(-c2cccc(N(c3ccccc3)c3ccc4c(oc5cc6ccccc6cc54)c3N(c3ccccc3)c3ccccc3)c2)cc1]

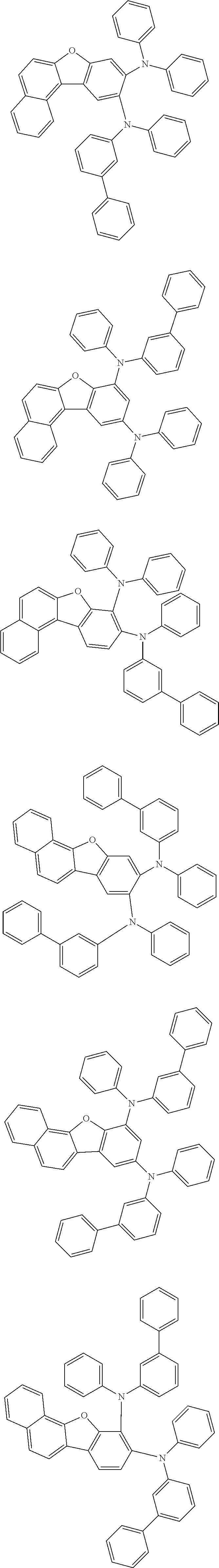

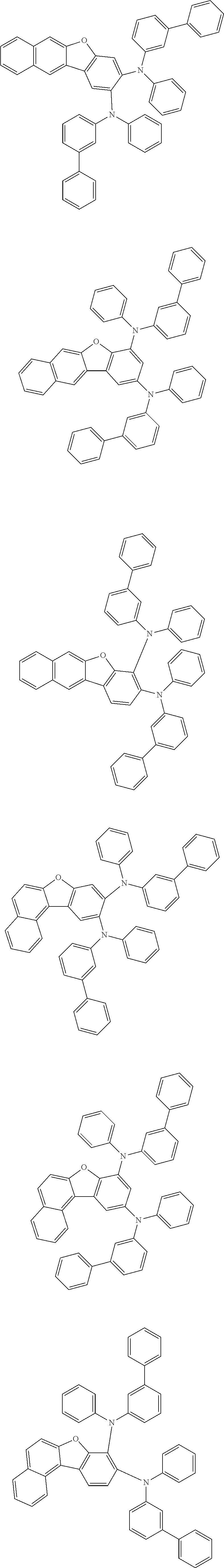

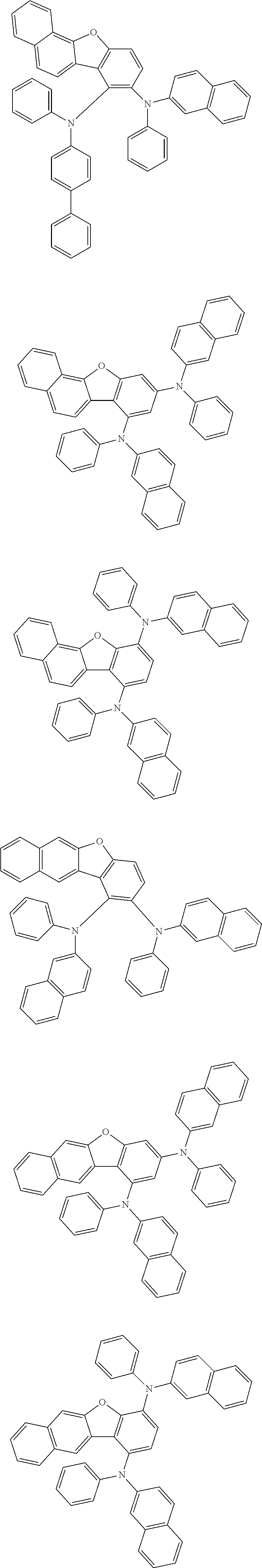 [SMILES: c1ccc(-c2ccc(N(c3ccccc3)c3c(N(c4ccccc4)c4ccc5ccccc5c4)ccc4oc5c6ccccc6ccc5c34)cc2)cc1 ; c1ccc(N(c2ccc3ccccc3c2)c2cc(N(c3ccccc3)c3ccc4ccccc4c3)c3c(c2)oc2c4ccccc4ccc23)cc1 ; c1ccc(N(c2ccc3ccccc3c2)c2ccc(N(c3ccccc3)c3ccc4ccccc4c3)c3c2oc2c4ccccc4ccc23)cc1 ; c1ccc(N(c2ccc3ccccc3c2)c2ccc3oc4cc5ccccc5cc4c3c2N(c2ccccc2)c2ccc3ccccc3c2)cc1 ; c1ccc(N(c2ccc3ccccc3c2)c2cc(N(c3ccccc3)c3ccc4ccccc4c3)c3c(c2)oc2cc4ccccc4cc23)cc1 ; c1ccc(N(c2ccc3ccccc3c2)c2ccc(N(c3ccccc3)c3ccc4ccccc4c3)c3c2oc2cc4ccccc4cc23)cc1]

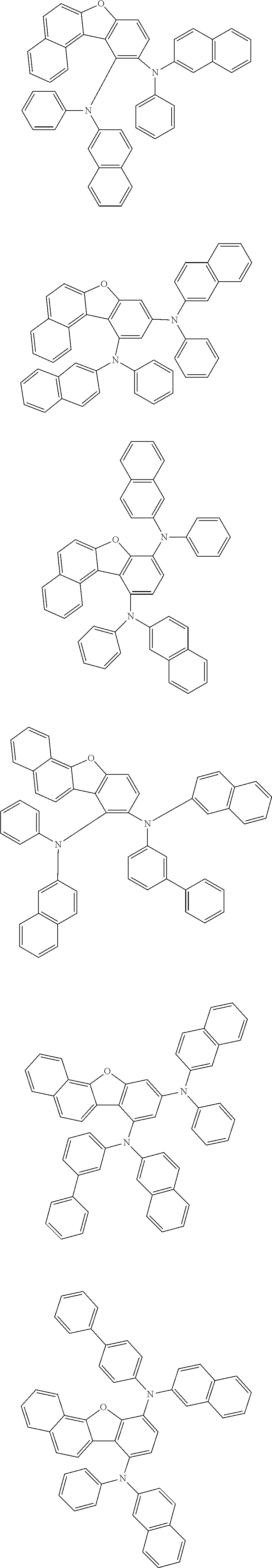

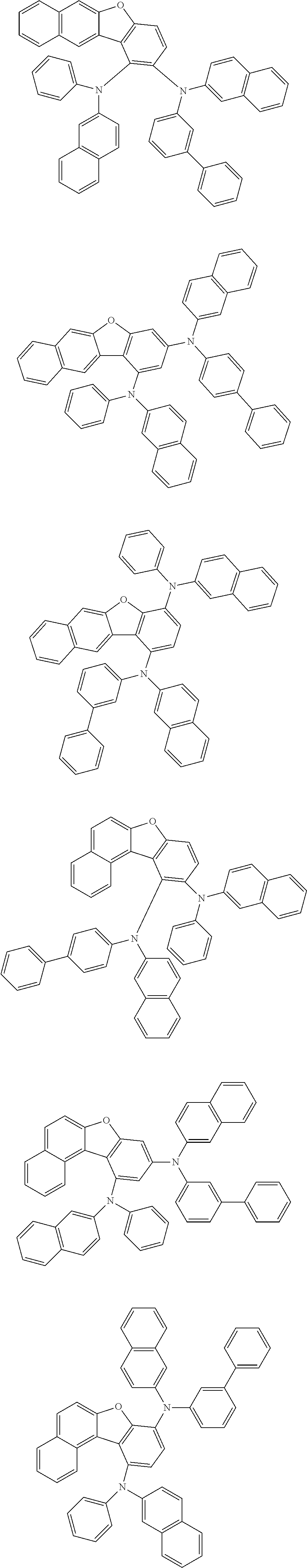

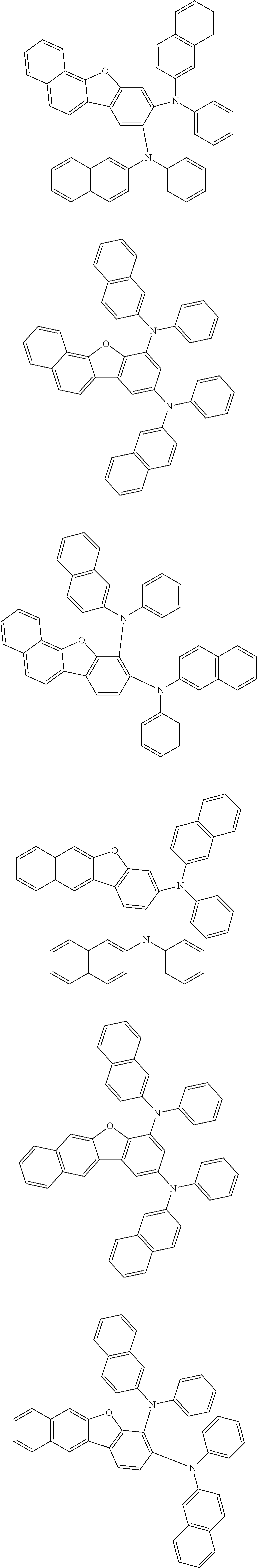 [SMILES: c1ccc(N(c2ccc3ccccc3c2)c2cc3oc4c5ccccc5ccc4c3cc2N(c2ccccc2)c2ccc3ccccc3c2)cc1 ; c1ccc(N(c2ccc3ccccc3c2)c2cc(N(c3ccccc3)c3ccc4ccccc4c3)c3oc4c5ccccc5ccc4c3c2)cc1 ; c1ccc(N(c2ccc3ccccc3c2)c2ccc3c(oc4c5ccccc5ccc34)c2N(c2ccccc2)c2ccc3ccccc3c2)cc1 ; c1ccc(N(c2ccc3ccccc3c2)c2cc3oc4cc5ccccc5cc4c3cc2N(c2ccccc2)c2ccc3ccccc3c2)cc1 ; c1ccc(N(c2ccc3ccccc3c2)c2cc(N(c3ccccc3)c3ccc4ccccc4c3)c3oc4cc5ccccc5cc4c3c2)cc1 ; c1ccc(N(c2ccc3ccccc3c2)c2ccc3c(oc4cc5ccccc5cc43)c2N(c2ccccc2)c2ccc3ccccc3c2)cc1]

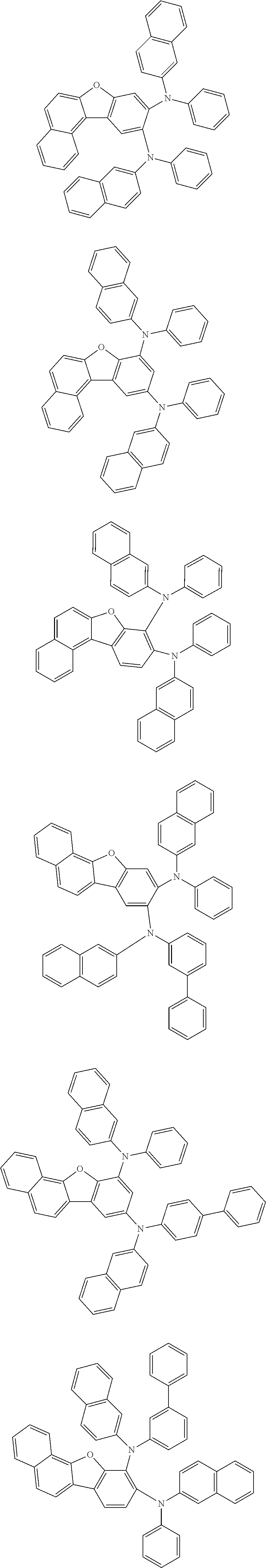

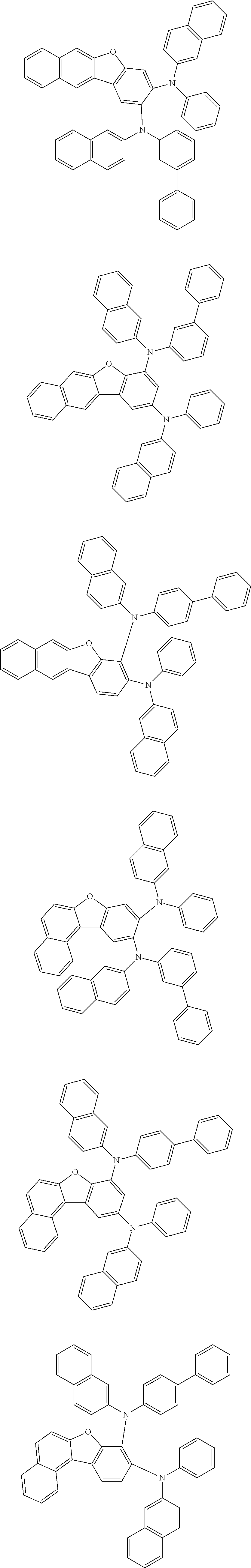

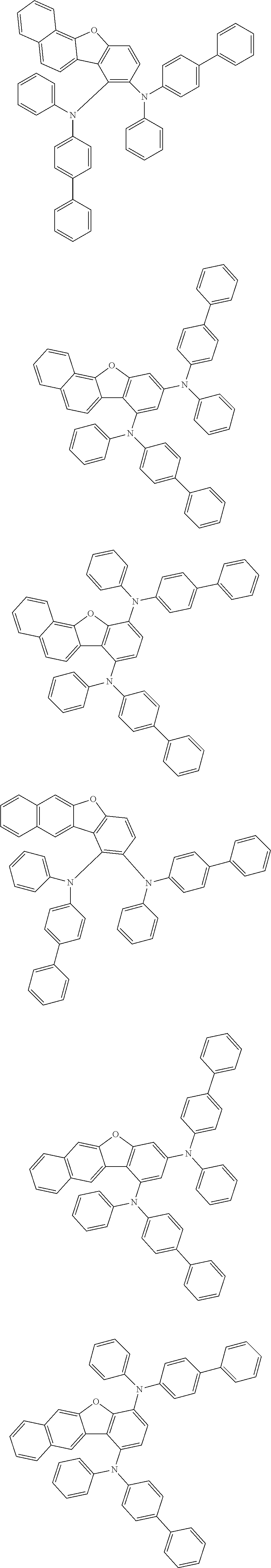

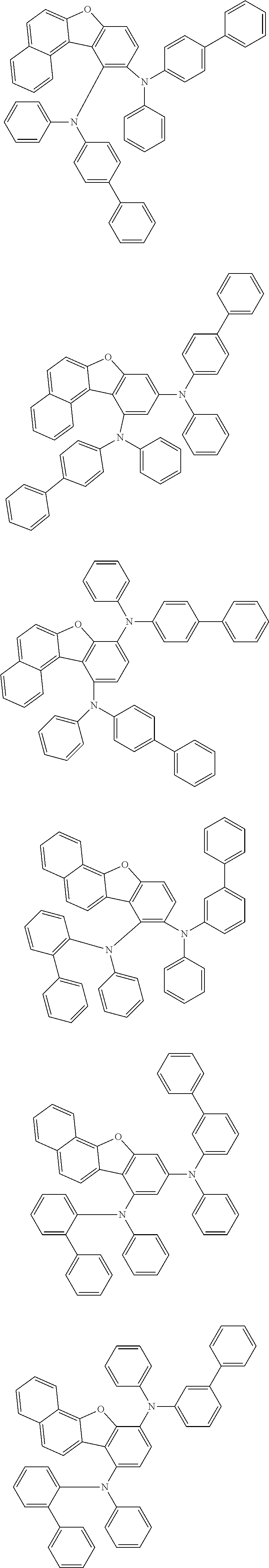

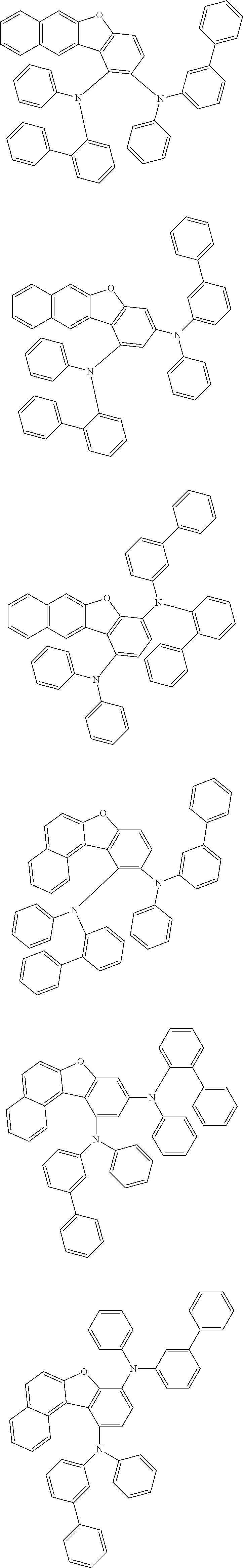

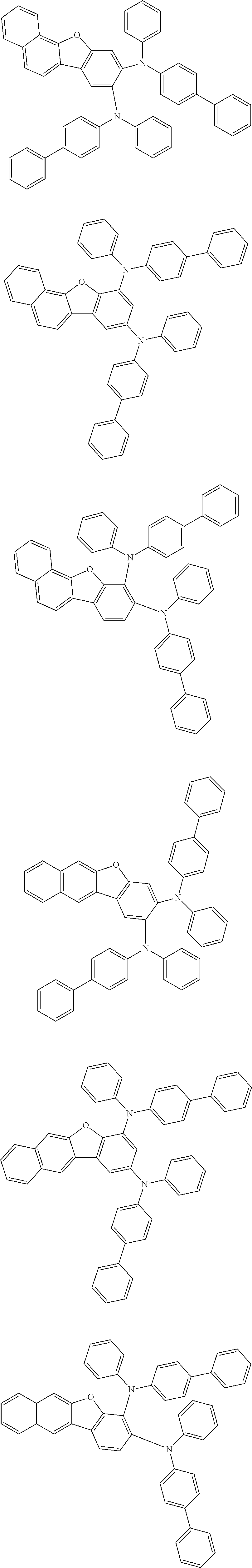

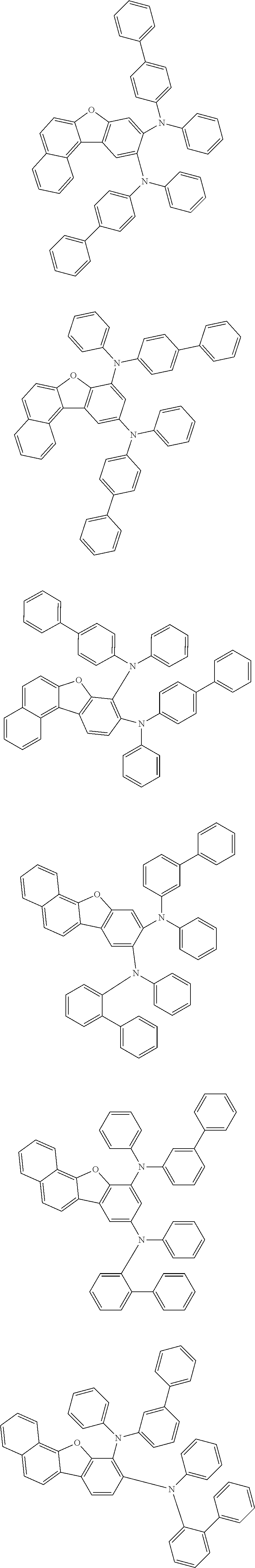

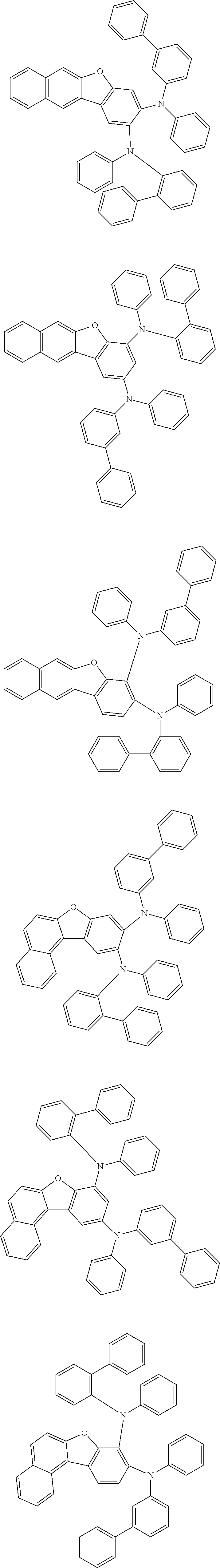

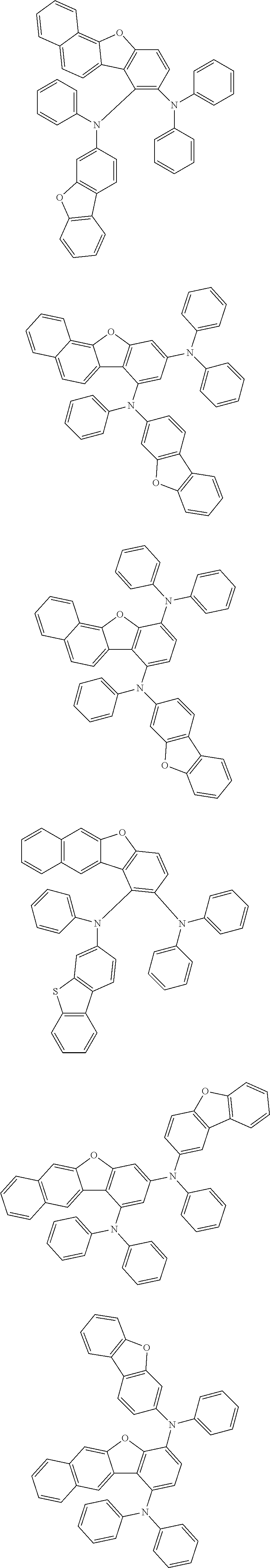 [SMILES: c1ccc(N(c2ccccc2)c2ccc3oc4c5ccccc5ccc4c3c2N(c2ccccc2)c2ccc3c(c2)oc2ccccc23)cc1 ; c1ccc(N(c2ccccc2)c2cc(N(c3ccccc3)c3ccc4c(c3)oc3ccccc34)c3c(c2)oc2c4ccccc4ccc23)cc1 ; c1ccc(N(c2ccccc2)c2ccc(N(c3ccccc3)c3ccc4c(c3)oc3ccccc34)c3c2oc2c4ccccc4ccc23)cc1 ; c1ccc(N(c2ccccc2)c2ccc3oc4cc5ccccc5cc4c3c2N(c2ccccc2)c2ccc3c(c2)sc2ccccc23)cc1 ; c1ccc(N(c2cc(N(c3ccccc3)c3ccccc3)c3c(c2)oc2cc4ccccc4cc23)c2ccc3oc4ccccc4c3c2)cc1 ; c1ccc(N(c2ccc3c(c2)oc2ccccc23)c2ccc(N(c3ccccc3)c3ccccc3)c3c2oc2cc4ccccc4cc23)cc1]

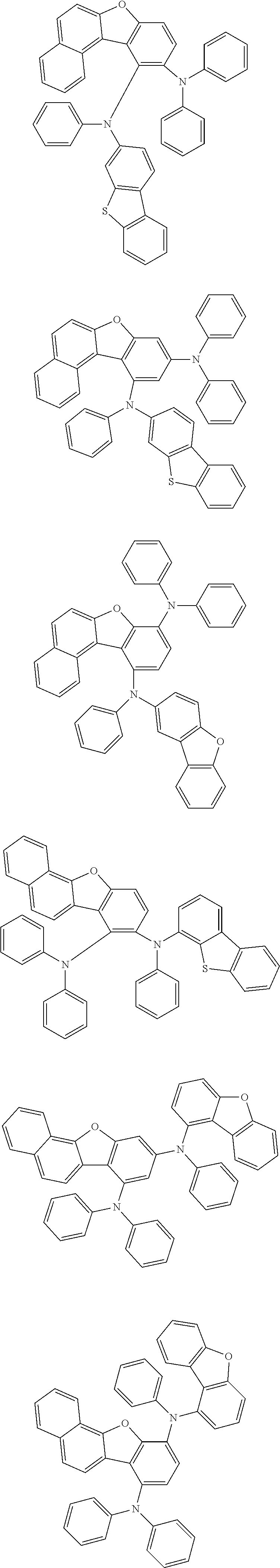 [SMILES: c1ccc(N(c2ccccc2)c2ccc3oc4ccc5ccccc5c4c3c2N(c2ccccc2)c2ccc3c(c2)sc2ccccc23)cc1 ; c1ccc(N(c2ccccc2)c2cc(N(c3ccccc3)c3ccc4c(c3)sc3ccccc34)c3c(c2)oc2ccc4ccccc4c23)cc1 ; c1ccc(N(c2ccccc2)c2ccc(N(c3ccccc3)c3ccc4oc5ccccc5c4c3)c3c2oc2ccc4ccccc4c23)cc1 ; c1ccc(N(c2ccc3oc4c5ccccc5ccc4c3c2N(c2ccccc2)c2ccccc2)c2cccc3c2sc2ccccc23)cc1 ; c1ccc(N(c2cc(N(c3ccccc3)c3ccccc3)c3c(c2)oc2c4ccccc4ccc23)c2cccc3oc4ccccc4c23)cc1 ; c1ccc(N(c2ccc(N(c3ccccc3)c3ccccc3)c3c2oc2c4ccccc4ccc23)c2cccc3oc4ccccc4c23)cc1]

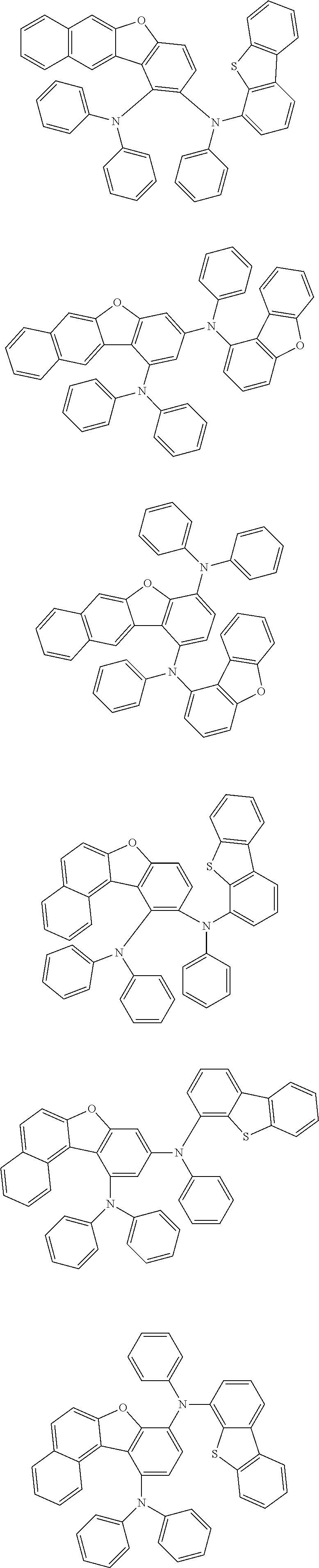 [SMILES: c1ccc(N(c2ccc3oc4cc5ccccc5cc4c3c2N(c2ccccc2)c2ccccc2)c2cccc3c2sc2ccccc23)cc1 ; c1ccc(N(c2cc(N(c3ccccc3)c3ccccc3)c3c(c2)oc2cc4ccccc4cc23)c2cccc3oc4ccccc4c23)cc1 ; c1ccc(N(c2ccccc2)c2ccc(N(c3ccccc3)c3cccc4oc5ccccc5c34)c3c2oc2cc4ccccc4cc23)cc1 ; c1ccc(N(c2ccc3oc4ccc5ccccc5c4c3c2N(c2ccccc2)c2ccccc2)c2cccc3c2sc2ccccc23)cc1 ; c1ccc(N(c2cc(N(c3ccccc3)c3ccccc3)c3c(c2)oc2ccc4ccccc4c23)c2cccc3c2sc2ccccc23)cc1 ; c1ccc(N(c2ccc(N(c3ccccc3)c3ccccc3)c3c2oc2ccc4ccccc4c23)c2cccc3c2sc2ccccc23)cc1]

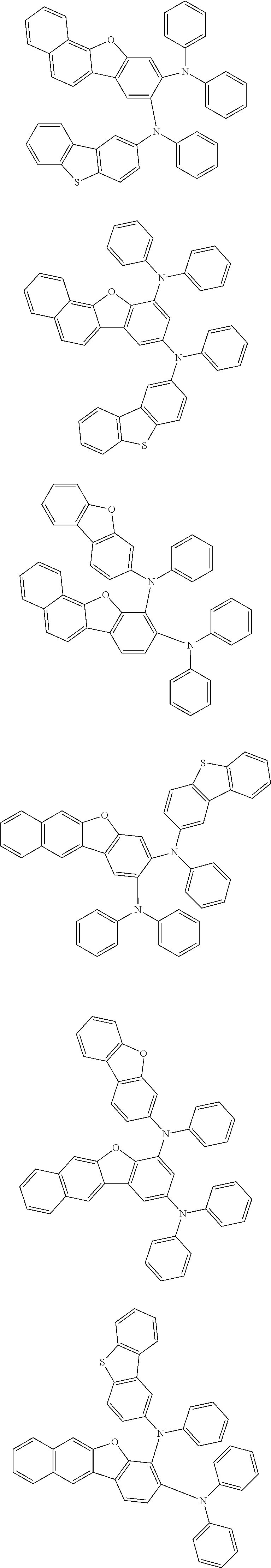 [SMILES: c1ccc(N(c2ccccc2)c2cc3oc4c5ccccc5ccc4c3cc2N(c2ccccc2)c2ccc3sc4ccccc4c3c2)cc1 ; c1ccc(N(c2ccc3sc4ccccc4c3c2)c2cc(N(c3ccccc3)c3ccccc3)c3oc4c5ccccc5ccc4c3c2)cc1 ; c1ccc(N(c2ccccc2)c2ccc3c(oc4c5ccccc5ccc34)c2N(c2ccccc2)c2ccc3c(c2)oc2ccccc23)cc1 ; c1ccc(N(c2ccc3sc4ccccc4c3c2)c2cc3oc4cc5ccccc5cc4c3cc2N(c2ccccc2)c2ccccc2)cc1 ; c1ccc(N(c2ccccc2)c2cc(N(c3ccccc3)c3ccc4c(c3)oc3ccccc34)c3oc4cc5ccccc5cc4c3c2)cc1 ; c1ccc(N(c2ccccc2)c2ccc3c(oc4cc5ccccc5cc43)c2N(c2ccccc2)c2ccc3sc4ccccc4c3c2)cc1]

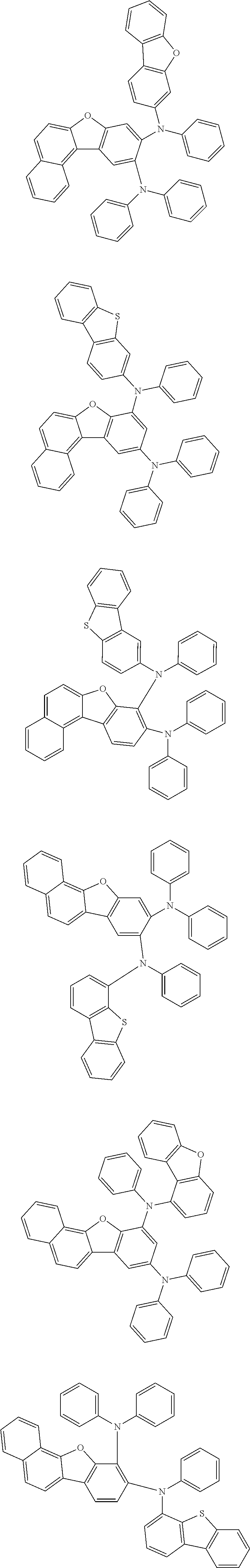 [SMILES: c1ccc(N(c2ccc3c(c2)oc2ccccc23)c2cc3oc4ccc5ccccc5c4c3cc2N(c2ccccc2)c2ccccc2)cc1 ; c1ccc(N(c2ccccc2)c2cc(N(c3ccccc3)c3ccc4c(c3)sc3ccccc34)c3oc4ccc5ccccc5c4c3c2)cc1 ; c1ccc(N(c2ccccc2)c2ccc3c(oc4ccc5ccccc5c43)c2N(c2ccccc2)c2ccc3sc4ccccc4c3c2)cc1 ; c1ccc(N(c2ccccc2)c2cc3oc4c5ccccc5ccc4c3cc2N(c2ccccc2)c2cccc3c2sc2ccccc23)cc1 ; c1ccc(N(c2ccccc2)c2cc(N(c3ccccc3)c3cccc4oc5ccccc5c34)c3oc4c5ccccc5ccc4c3c2)cc1 ; c1ccc(N(c2ccccc2)c2c(N(c3ccccc3)c3cccc4c3sc3ccccc34)ccc3c2oc2c4ccccc4ccc32)cc1]

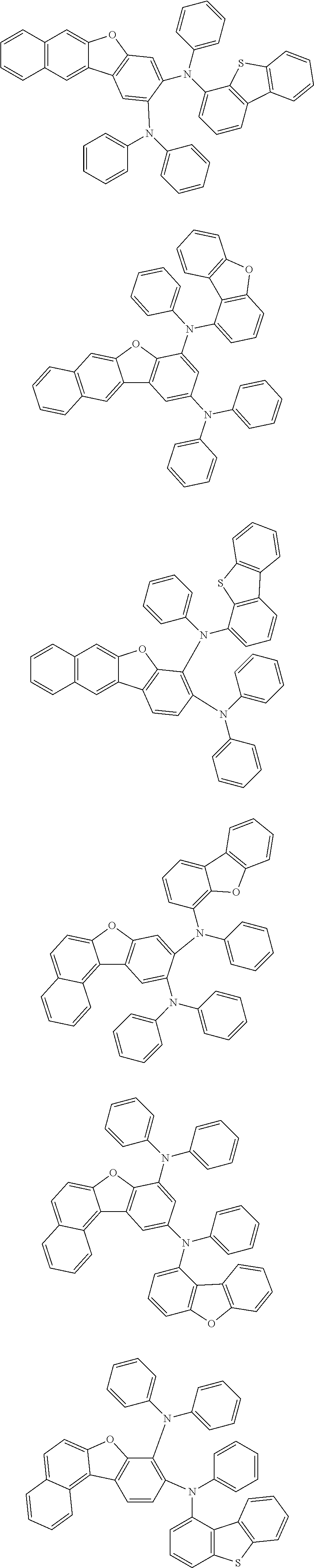

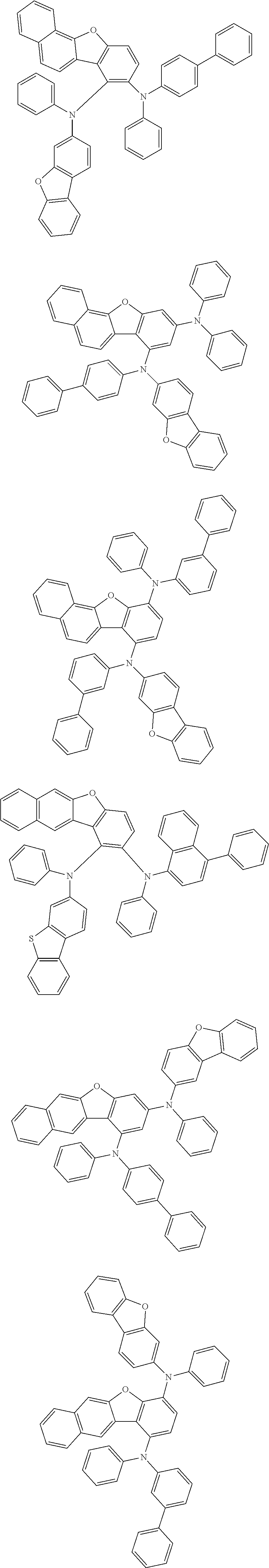

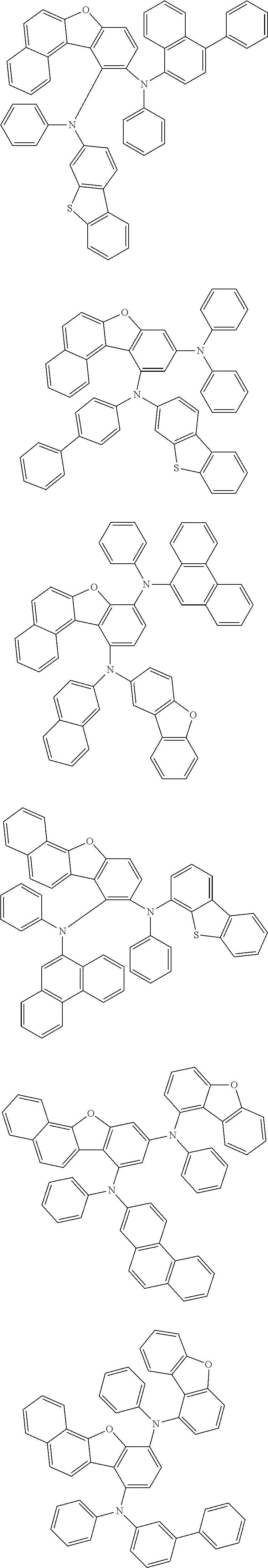

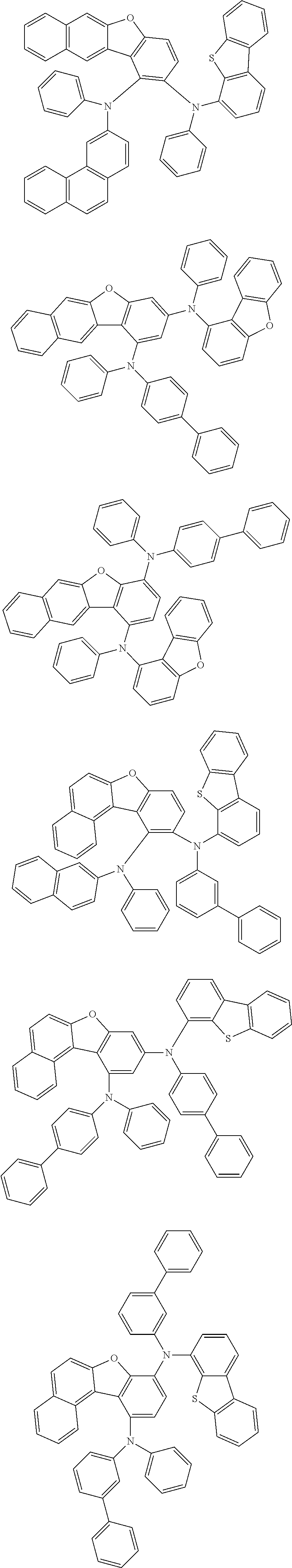

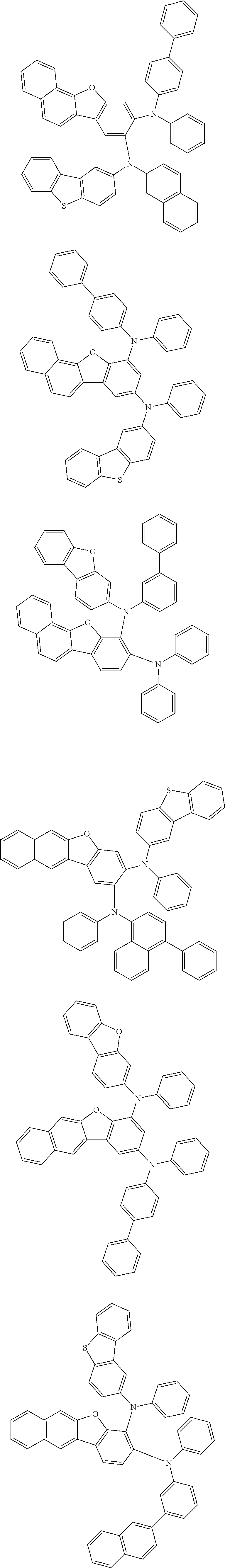

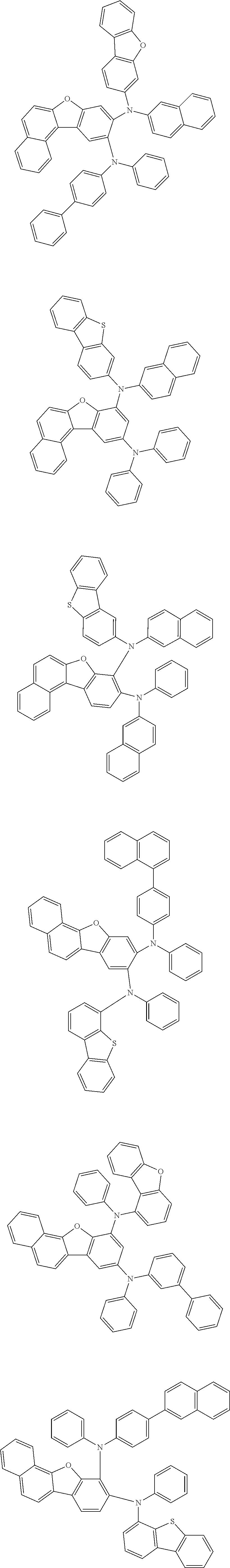

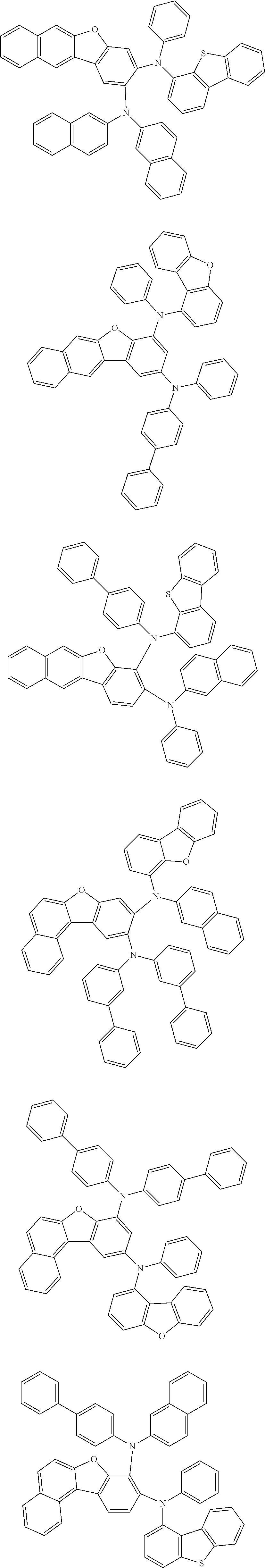

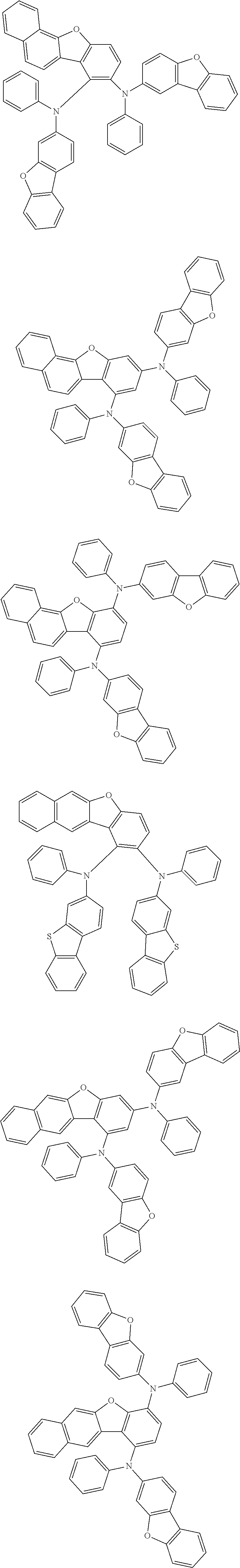

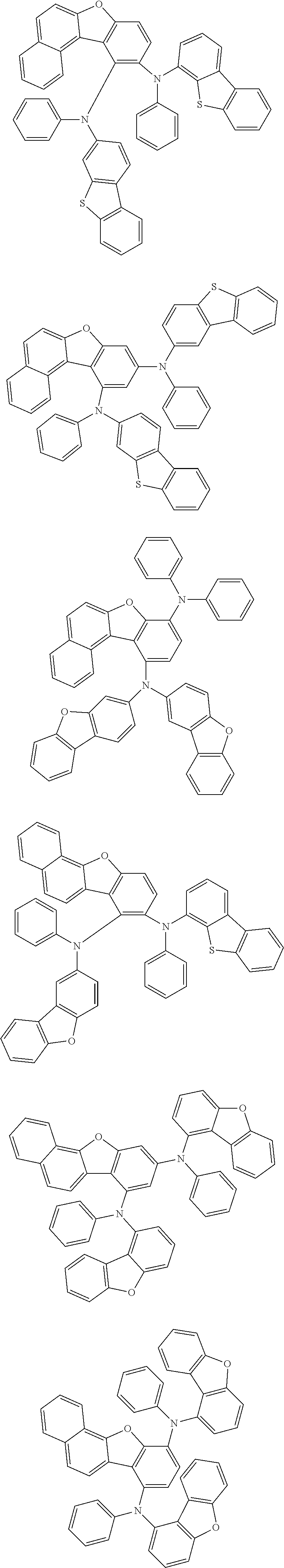

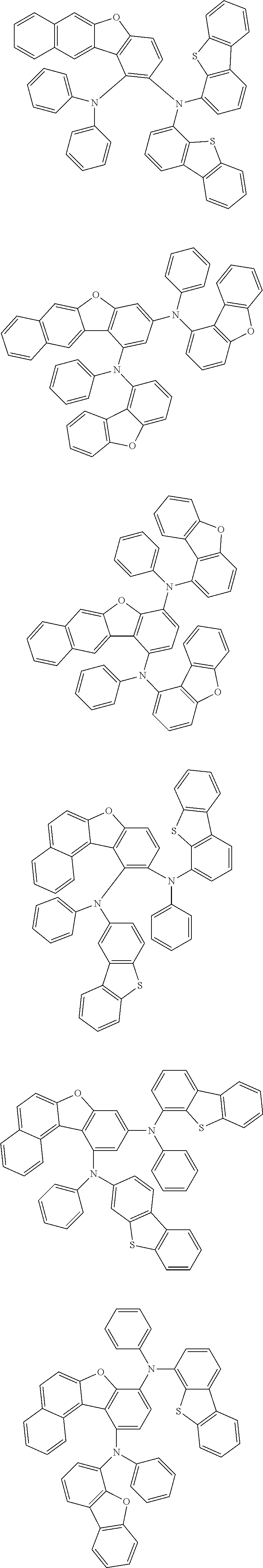

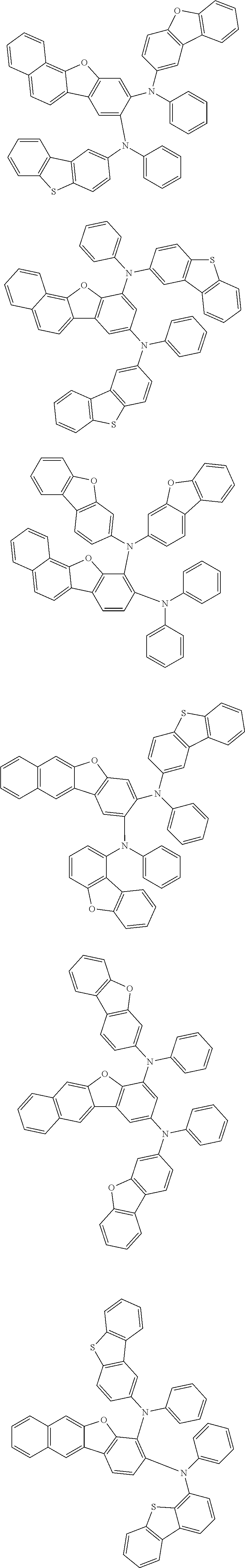

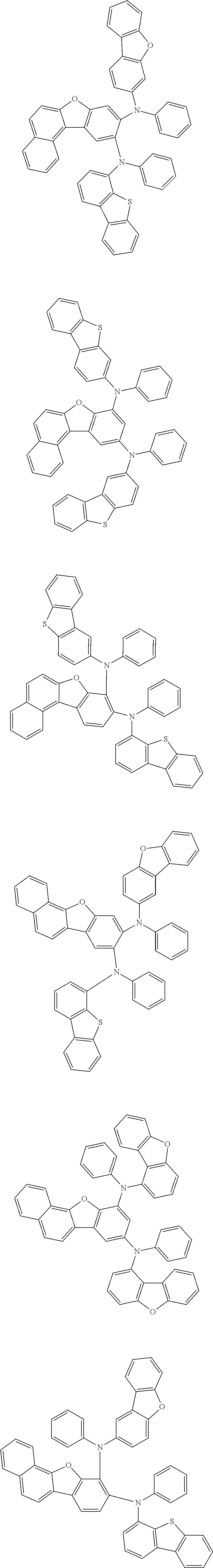

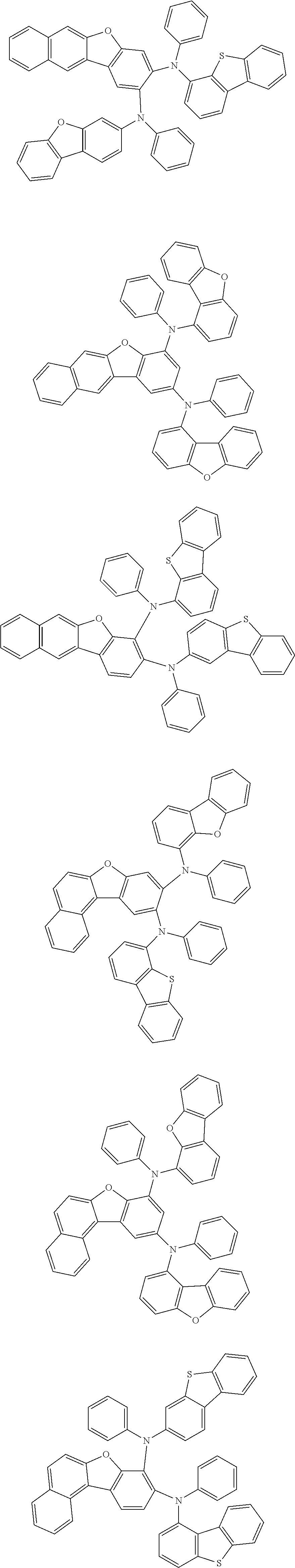

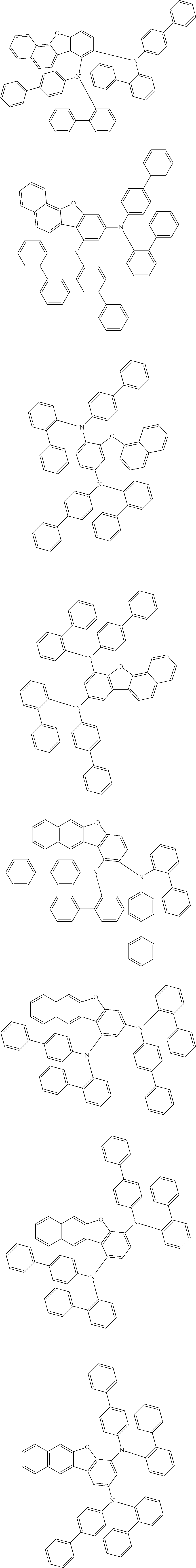

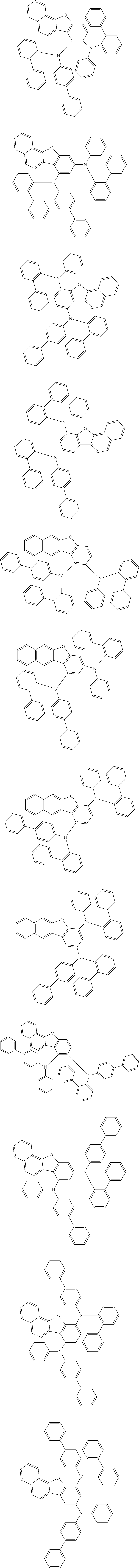

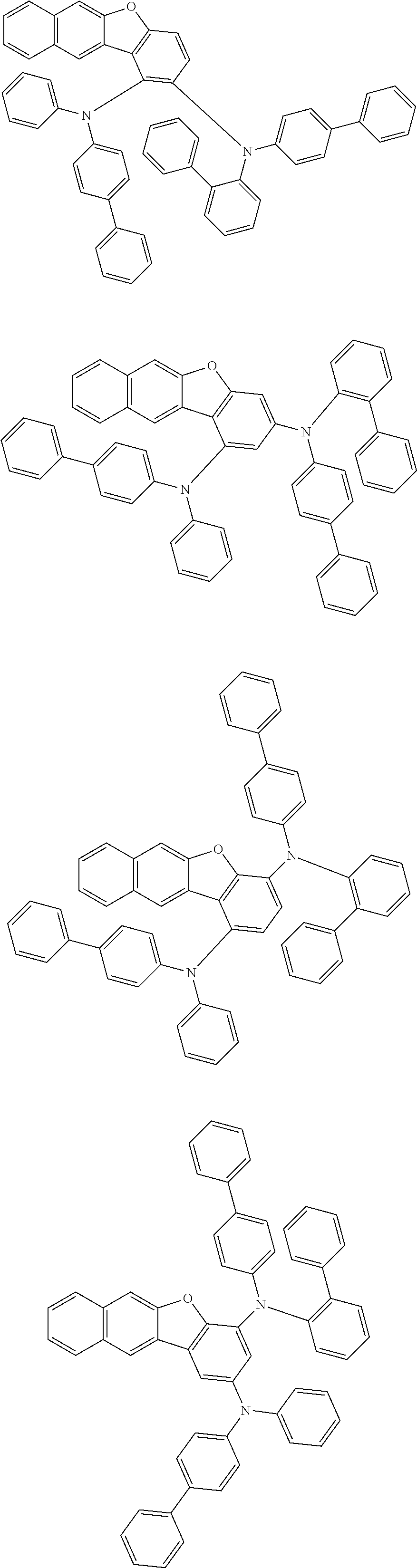 [SMILES: c1ccc(-c2ccc(N(c3ccccc3-c3ccccc3)c3ccc4oc5cc6ccccc6cc5c4c3N(c3ccccc3)c3ccc(-c4ccccc4)cc3)cc2)cc1 ; c1ccc(-c2ccc(N(c3cc(N(c4ccccc4)c4ccc(-c5ccccc5)cc4)c4c(c3)oc3cc5ccccc5cc34)c3ccccc3-c3ccccc3)cc2)cc1 ; c1ccc(-c2ccc(N(c3ccccc3-c3ccccc3)c3ccc(N(c4ccccc4)c4ccc(-c5ccccc5)cc4)c4c3oc3cc5ccccc5cc34)cc2)cc1 ; c1ccc(-c2ccc(N(c3ccccc3)c3cc(N(c4ccc(-c5ccccc5)cc4)c4ccccc4-c4ccccc4)c4oc5cc6ccccc6cc5c4c3)cc2)cc1]

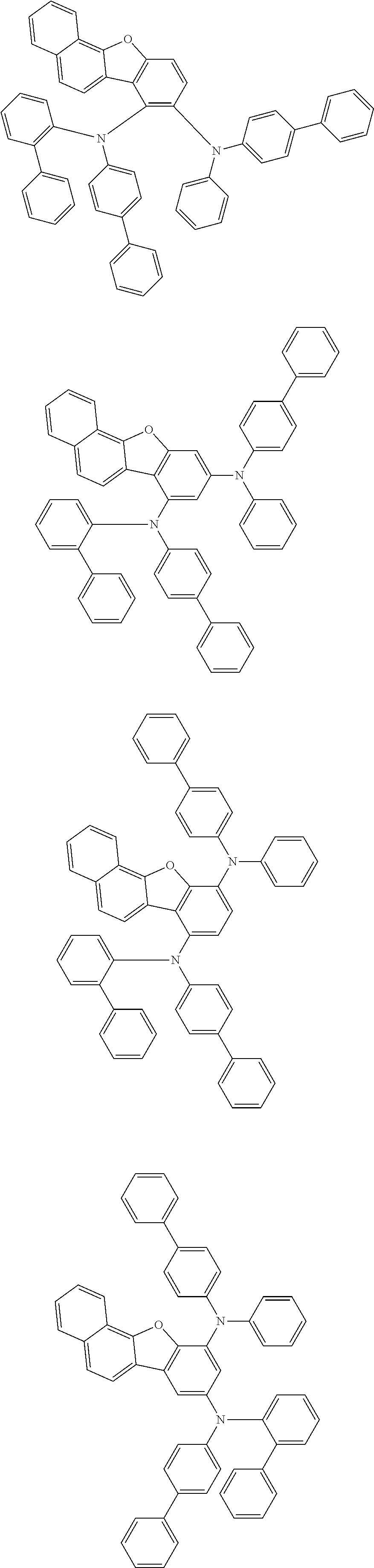 [SMILES: c1ccc(-c2ccc(N(c3ccccc3)c3ccc4oc5c6ccccc6ccc5c4c3N(c3ccc(-c4ccccc4)cc3)c3ccccc3-c3ccccc3)cc2)cc1 ; c1ccc(-c2ccc(N(c3ccccc3)c3cc(N(c4ccc(-c5ccccc5)cc4)c4ccccc4-c4ccccc4)c4c(c3)oc3c5ccccc5ccc34)cc2)cc1 ; c1ccc(-c2ccc(N(c3ccccc3)c3ccc(N(c4ccc(-c5ccccc5)cc4)c4ccccc4-c4ccccc4)c4c3oc3c5ccccc5ccc34)cc2)cc1 ; c1ccc(-c2ccc(N(c3cc(N(c4ccccc4)c4ccc(-c5ccccc5)cc4)c4oc5c6ccccc6ccc5c4c3)c3ccccc3-c3ccccc3)cc2)cc1]

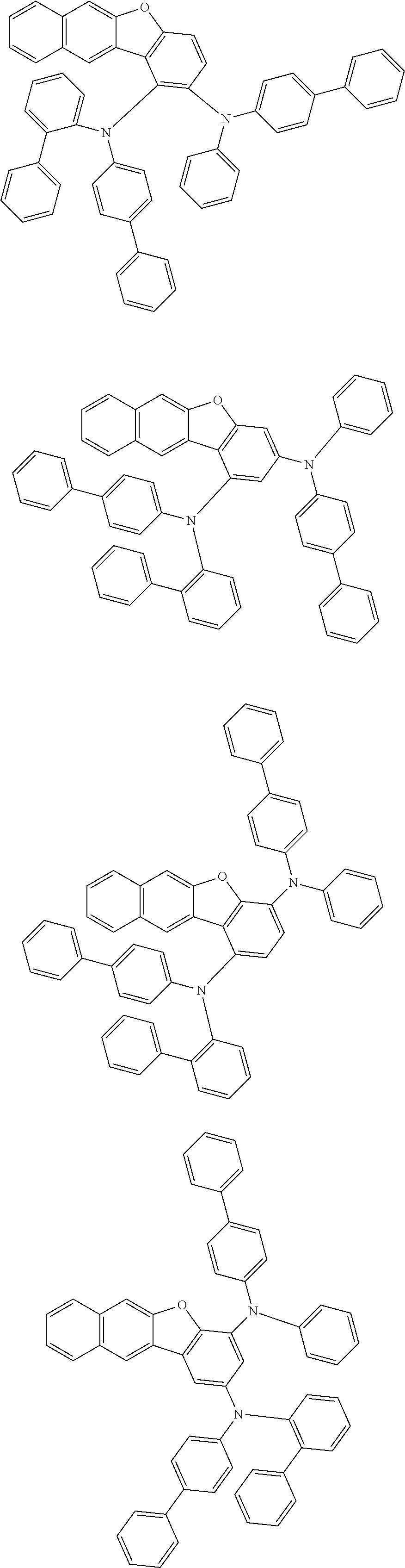

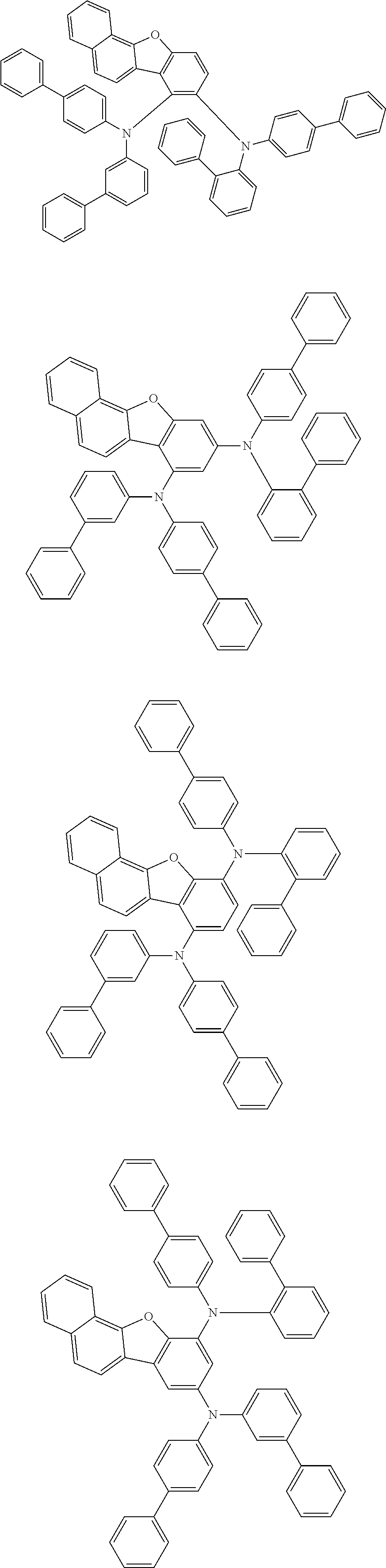 [SMILES: c1ccc(-c2ccc(N(c3ccccc3-c3ccccc3)c3ccc4oc5c6ccccc6ccc5c4c3N(c3ccc(-c4ccccc4)cc3)c3cccc(-c4ccccc4)c3)cc2)cc1 ; c1ccc(-c2ccc(N(c3cc(N(c4ccc(-c5ccccc5)cc4)c4cccc(-c5ccccc5)c4)c4c(c3)oc3c5ccccc5ccc34)c3ccccc3-c3ccccc3)cc2)cc1 ; c1ccc(-c2ccc(N(c3ccccc3-c3ccccc3)c3ccc(N(c4ccc(-c5ccccc5)cc4)c4cccc(-c5ccccc5)c4)c4c3oc3c5ccccc5ccc34)cc2)cc1 ; c1ccc(-c2ccc(N(c3cccc(-c4ccccc4)c3)c3cc(N(c4ccc(-c5ccccc5)cc4)c4ccccc4-c4ccccc4)c4oc5c6ccccc6ccc5c4c3)cc2)cc1]

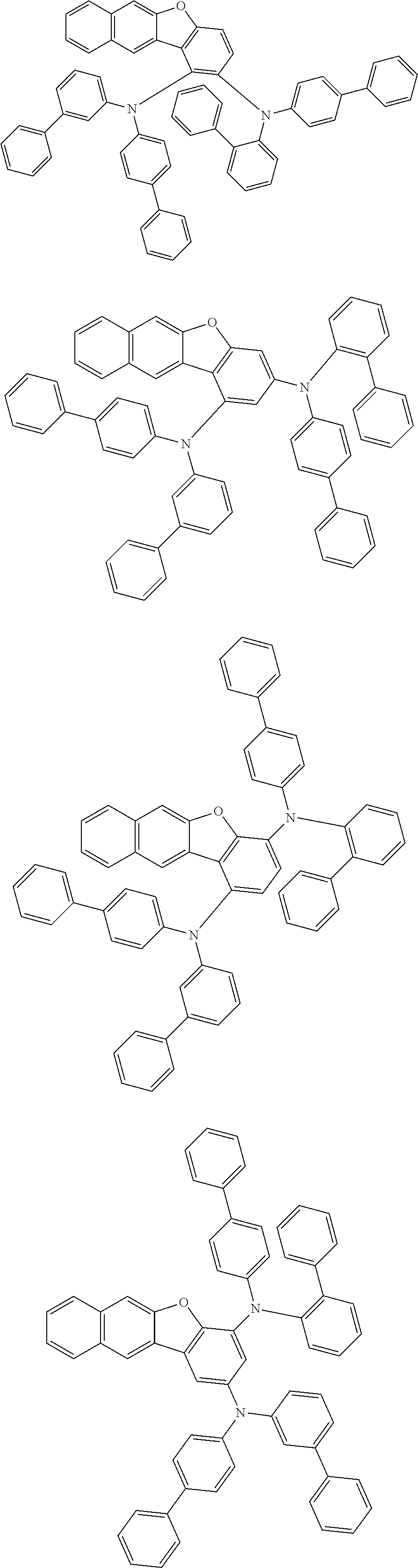 [SMILES: c1ccc(-c2ccc(N(c3ccccc3-c3ccccc3)c3ccc4oc5cc6ccccc6cc5c4c3N(c3ccc(-c4ccccc4)cc3)c3cccc(-c4ccccc4)c3)cc2)cc1 ; c1ccc(-c2ccc(N(c3cc(N(c4ccc(-c5ccccc5)cc4)c4cccc(-c5ccccc5)c4)c4c(c3)oc3cc5ccccc5cc34)c3ccccc3-c3ccccc3)cc2)cc1 ; c1ccc(-c2ccc(N(c3ccccc3-c3ccccc3)c3ccc(N(c4ccc(-c5ccccc5)cc4)c4cccc(-c5ccccc5)c4)c4c3oc3cc5ccccc5cc34)cc2)cc1 ; c1ccc(-c2ccc(N(c3cccc(-c4ccccc4)c3)c3cc(N(c4ccc(-c5ccccc5)cc4)c4ccccc4-c4ccccc4)c4oc5cc6ccccc6cc5c4c3)cc2)cc1]

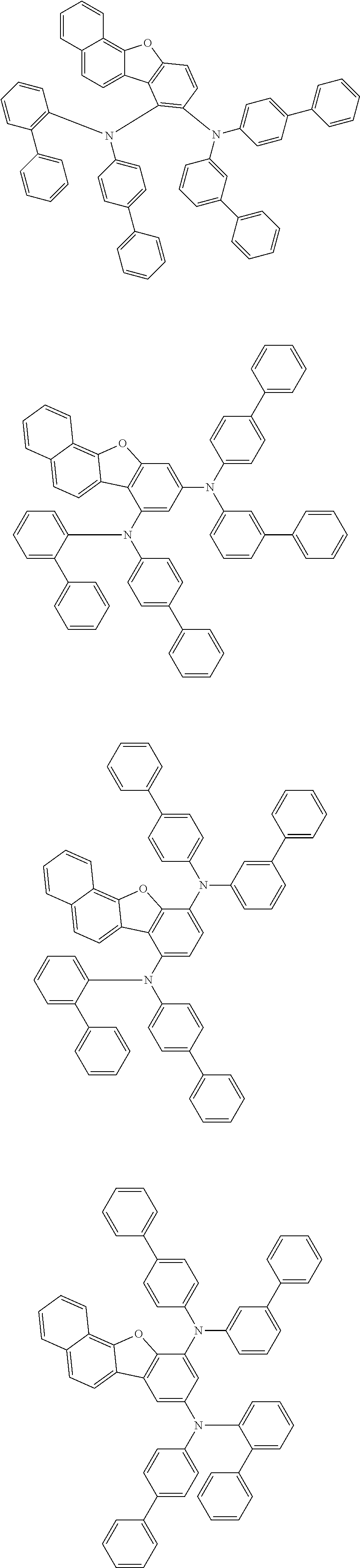 [SMILES: c1ccc(-c2ccc(N(c3cccc(-c4ccccc4)c3)c3ccc4oc5c6ccccc6ccc5c4c3N(c3ccc(-c4ccccc4)cc3)c3ccccc3-c3ccccc3)cc2)cc1 ; c1ccc(-c2ccc(N(c3cccc(-c4ccccc4)c3)c3cc(N(c4ccc(-c5ccccc5)cc4)c4ccccc4-c4ccccc4)c4c(c3)oc3c5ccccc5ccc34)cc2)cc1 ; c1ccc(-c2ccc(N(c3cccc(-c4ccccc4)c3)c3ccc(N(c4ccc(-c5ccccc5)cc4)c4ccccc4-c4ccccc4)c4c3oc3c5ccccc5ccc34)cc2)cc1 ; c1ccc(-c2ccc(N(c3cc(N(c4ccc(-c5ccccc5)cc4)c4cccc(-c5ccccc5)c4)c4oc5c6ccccc6ccc5c4c3)c3ccccc3-c3ccccc3)cc2)cc1]

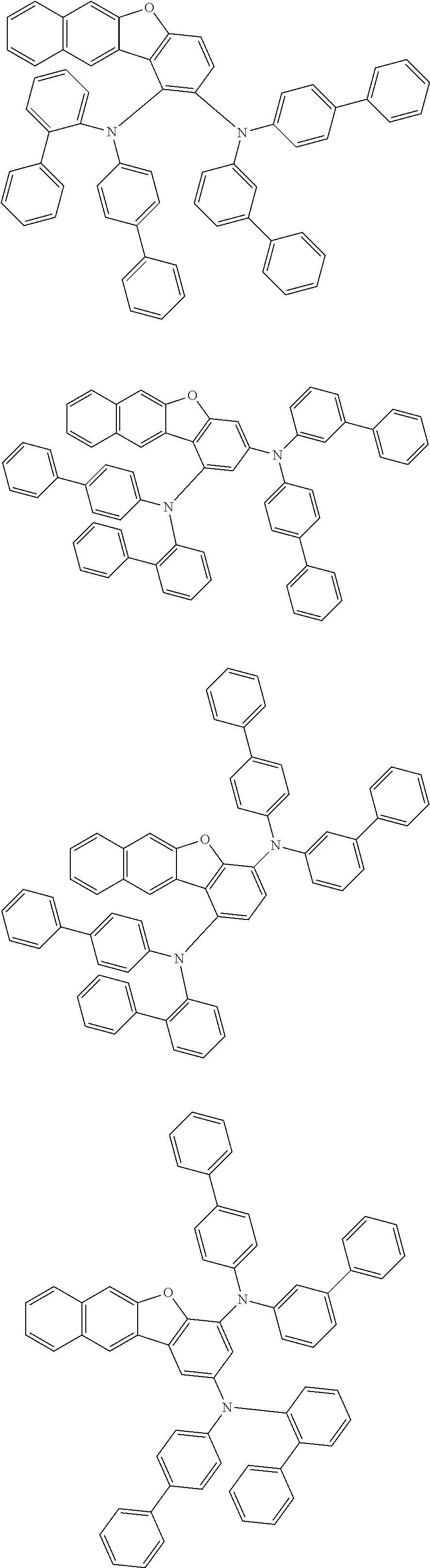 [SMILES: c1ccc(-c2ccc(N(c3cccc(-c4ccccc4)c3)c3ccc4oc5cc6ccccc6cc5c4c3N(c3ccc(-c4ccccc4)cc3)c3ccccc3-c3ccccc3)cc2)cc1 ; c1ccc(-c2ccc(N(c3cccc(-c4ccccc4)c3)c3cc(N(c4ccc(-c5ccccc5)cc4)c4ccccc4-c4ccccc4)c4c(c3)oc3cc5ccccc5cc34)cc2)cc1 ; c1ccc(-c2ccc(N(c3cccc(-c4ccccc4)c3)c3ccc(N(c4ccc(-c5ccccc5)cc4)c4ccccc4-c4ccccc4)c4c3oc3cc5ccccc5cc34)cc2)cc1 ; c1ccc(-c2ccc(N(c3cc(N(c4ccc(-c5ccccc5)cc4)c4cccc(-c5ccccc5)c4)c4oc5cc6ccccc6cc5c4c3)c3ccccc3-c3ccccc3)cc2)cc1]

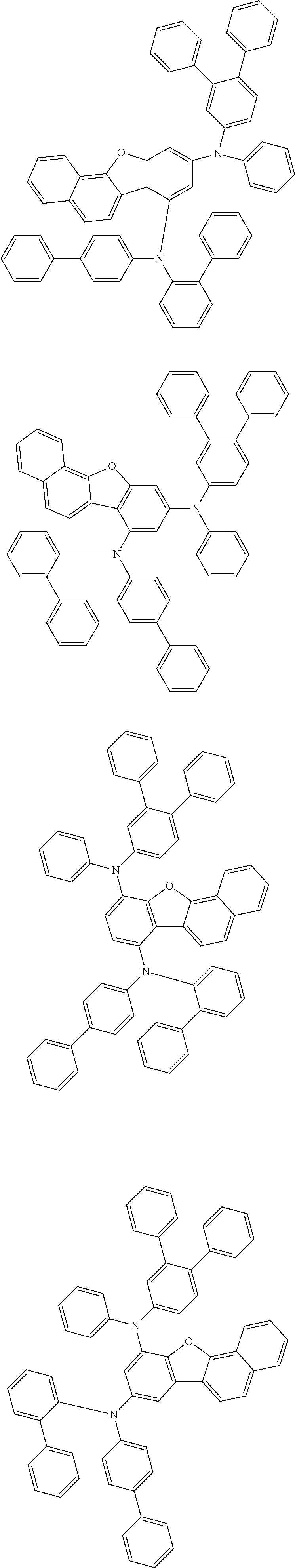 [SMILES: c1ccc(-c2ccc(N(c3ccccc3-c3ccccc3)c3cc(N(c4ccccc4)c4ccc(-c5ccccc5)c(-c5ccccc5)c4)cc4oc5c6ccccc6ccc5c34)cc2)cc1 ; c1ccc(-c2ccc(N(c3ccccc3-c3ccccc3)c3cc(N(c4ccccc4)c4ccc(-c5ccccc5)c(-c5ccccc5)c4)cc4oc5c6ccccc6ccc5c34)cc2)cc1 ; c1ccc(-c2ccc(N(c3ccccc3-c3ccccc3)c3ccc(N(c4ccccc4)c4ccc(-c5ccccc5)c(-c5ccccc5)c4)c4oc5c6ccccc6ccc5c34)cc2)cc1 ; c1ccc(-c2ccc(N(c3cc(N(c4ccccc4)c4ccc(-c5ccccc5)c(-c5ccccc5)c4)c4oc5c6ccccc6ccc5c4c3)c3ccccc3-c3ccccc3)cc2)cc1]

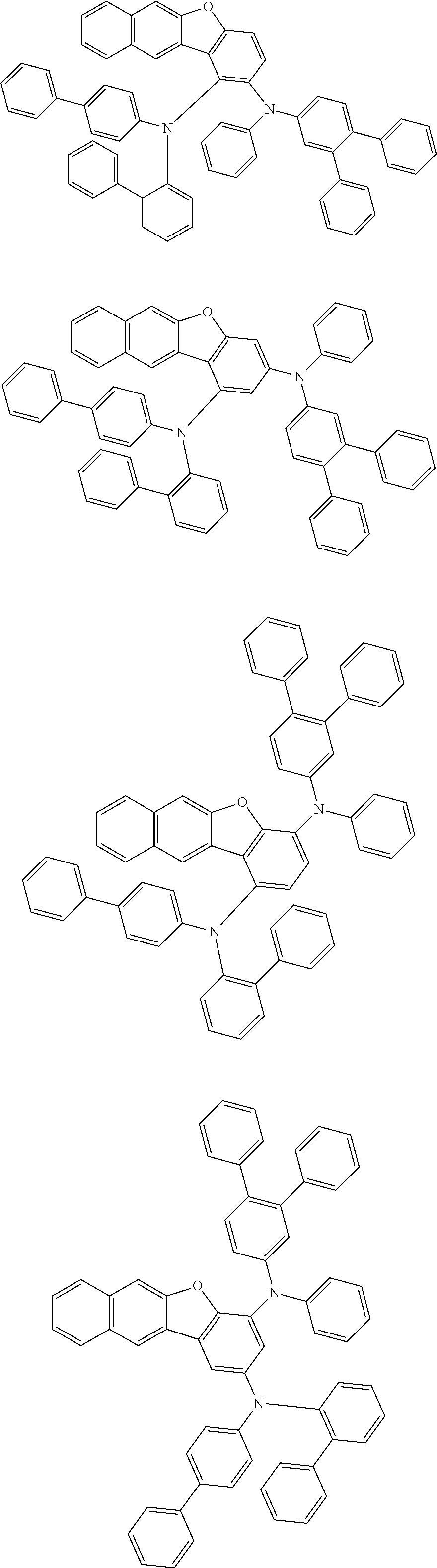 [SMILES: c1ccc(-c2ccc(N(c3ccccc3-c3ccccc3)c3c(N(c4ccccc4)c4ccc(-c5ccccc5)c(-c5ccccc5)c4)ccc4oc5cc6ccccc6cc5c34)cc2)cc1 ; c1ccc(-c2ccc(N(c3ccccc3-c3ccccc3)c3cc(N(c4ccccc4)c4ccc(-c5ccccc5)c(-c5ccccc5)c4)cc4oc5cc6ccccc6cc5c34)cc2)cc1 ; c1ccc(-c2ccc(N(c3ccccc3-c3ccccc3)c3ccc(N(c4ccccc4)c4ccc(-c5ccccc5)c(-c5ccccc5)c4)c4oc5cc6ccccc6cc5c34)cc2)cc1 ; c1ccc(-c2ccc(N(c3cc(N(c4ccccc4)c4ccc(-c5ccccc5)c(-c5ccccc5)c4)c4oc5cc6ccccc6cc5c4c3)c3ccccc3-c3ccccc3)cc2)cc1]

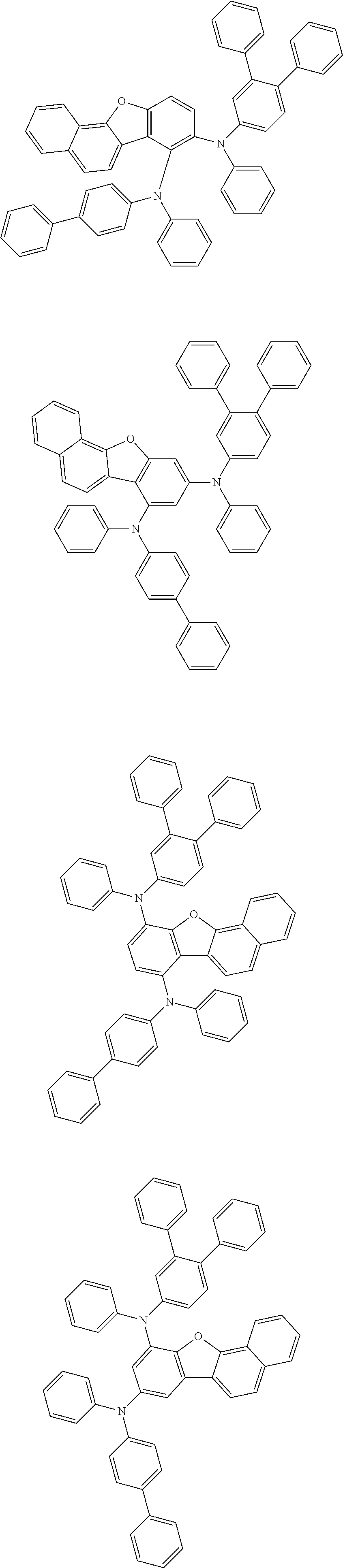 [SMILES: c1ccc(-c2ccc(N(c3ccccc3)c3c(N(c4ccccc4)c4ccc(-c5ccccc5)c(-c5ccccc5)c4)ccc4oc5c6ccccc6ccc5c34)cc2)cc1 ; c1ccc(-c2ccc(N(c3ccccc3)c3cc(N(c4ccccc4)c4ccc(-c5ccccc5)c(-c5ccccc5)c4)cc4oc5c6ccccc6ccc5c34)cc2)cc1 ; c1ccc(-c2ccc(N(c3ccccc3)c3ccc(N(c4ccccc4)c4ccc(-c5ccccc5)c(-c5ccccc5)c4)c4oc5c6ccccc6ccc5c34)cc2)cc1 ; c1ccc(-c2ccc(N(c3ccccc3)c3cc(N(c4ccccc4)c4ccc(-c5ccccc5)c(-c5ccccc5)c4)c4oc5c6ccccc6ccc5c4c3)cc2)cc1]

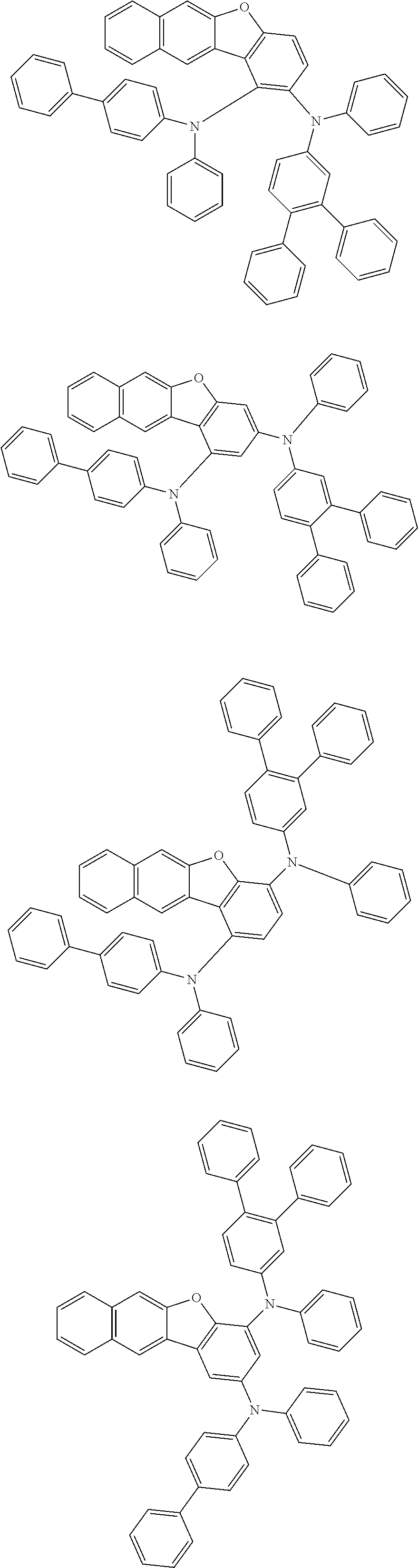 [SMILES: c1ccc(-c2ccc(N(c3ccccc3)c3c(N(c4ccccc4)c4ccc(-c5ccccc5)c(-c5ccccc5)c4)ccc4oc5cc6ccccc6cc5c34)cc2)cc1 ; c1ccc(-c2ccc(N(c3ccccc3)c3cc(N(c4ccccc4)c4ccc(-c5ccccc5)c(-c5ccccc5)c4)cc4oc5cc6ccccc6cc5c34)cc2)cc1 ; c1ccc(-c2ccc(N(c3ccccc3)c3ccc(N(c4ccccc4)c4ccc(-c5ccccc5)c(-c5ccccc5)c4)c4oc5cc6ccccc6cc5c34)cc2)cc1 ; c1ccc(-c2ccc(N(c3ccccc3)c3cc(N(c4ccccc4)c4ccc(-c5ccccc5)c(-c5ccccc5)c4)c4oc5cc6ccccc6cc5c4c3)cc2)cc1]

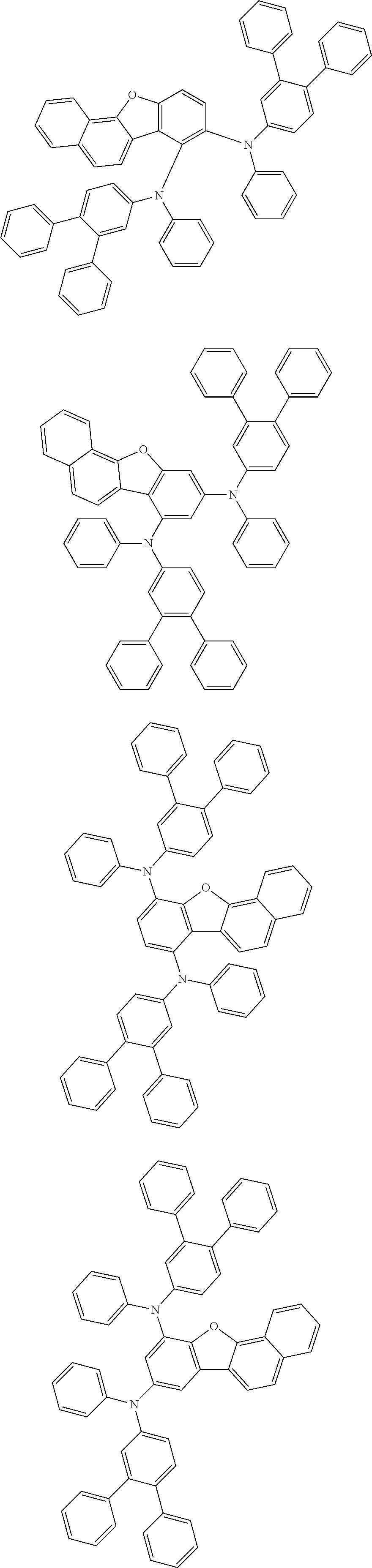

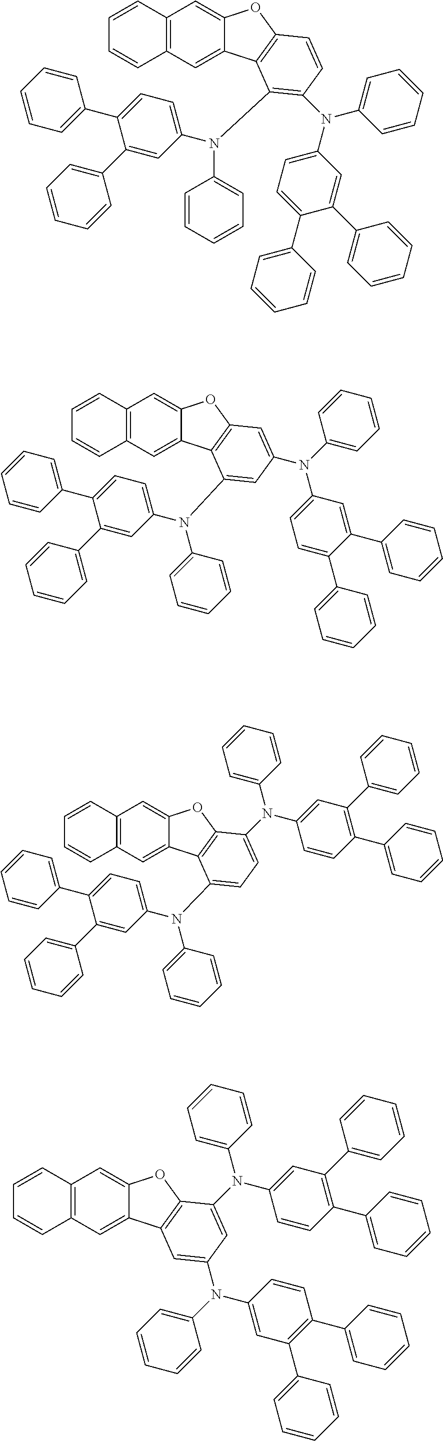 [SMILES: c1ccc(-c2ccc(N(c3ccccc3)c3ccc4oc5cc6ccccc6cc5c4c3N(c3ccccc3)c3ccc(-c4ccccc4)c(-c4ccccc4)c3)cc2-c2ccccc2)cc1 ; c1ccc(-c2ccc(N(c3ccccc3)c3cc(N(c4ccccc4)c4ccc(-c5ccccc5)c(-c5ccccc5)c4)c4c(c3)oc3cc5ccccc5cc34)cc2-c2ccccc2)cc1 ; c1ccc(-c2ccc(N(c3ccccc3)c3ccc(N(c4ccccc4)c4ccc(-c5ccccc5)c(-c5ccccc5)c4)c4c3oc3cc5ccccc5cc34)cc2-c2ccccc2)cc1 ; c1ccc(-c2ccc(N(c3ccccc3)c3cc(N(c4ccccc4)c4ccc(-c5ccccc5)c(-c5ccccc5)c4)c4oc5cc6ccccc6cc5c4c3)cc2-c2ccccc2)cc1]

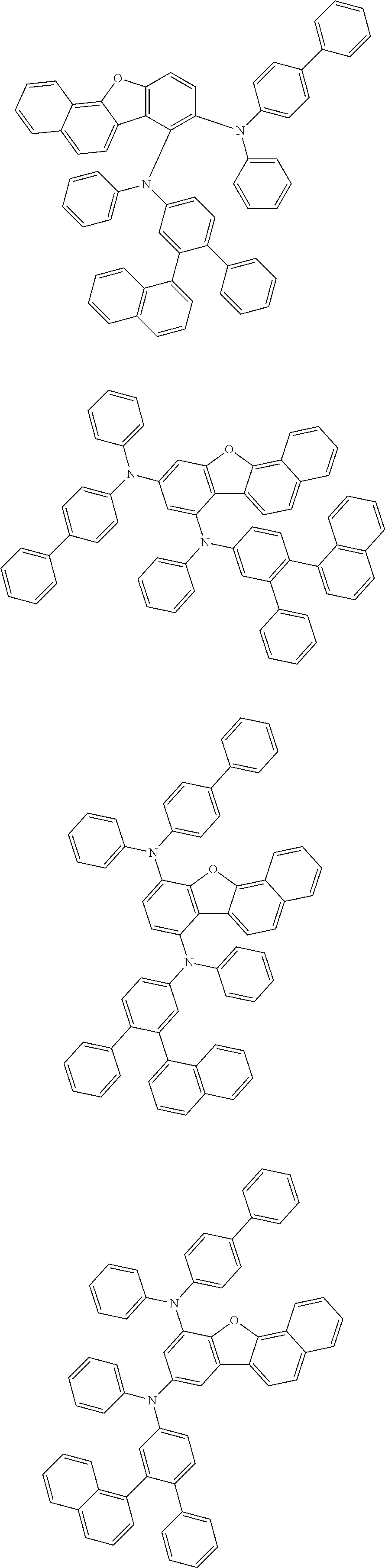 [SMILES: c1ccc(-c2ccc(N(c3ccccc3)c3ccc4oc5c6ccccc6ccc5c4c3N(c3ccccc3)c3ccc(-c4ccccc4)c(-c4cccc5ccccc45)c3)cc2)cc1 ; c1ccc(-c2ccc(N(c3ccccc3)c3cc(N(c4ccccc4)c4ccc(-c5cccc6ccccc56)c(-c5ccccc5)c4)c4c(c3)oc3c5ccccc5ccc34)cc2)cc1 ; c1ccc(-c2ccc(N(c3ccccc3)c3ccc(N(c4ccccc4)c4ccc(-c5ccccc5)c(-c5cccc6ccccc56)c4)c4c3oc3c5ccccc5ccc34)cc2)cc1 ; c1ccc(-c2ccc(N(c3ccccc3)c3cc(N(c4ccccc4)c4ccc(-c5ccccc5)c(-c5cccc6ccccc56)c4)cc4c3oc3c5ccccc5ccc43)cc2)cc1]

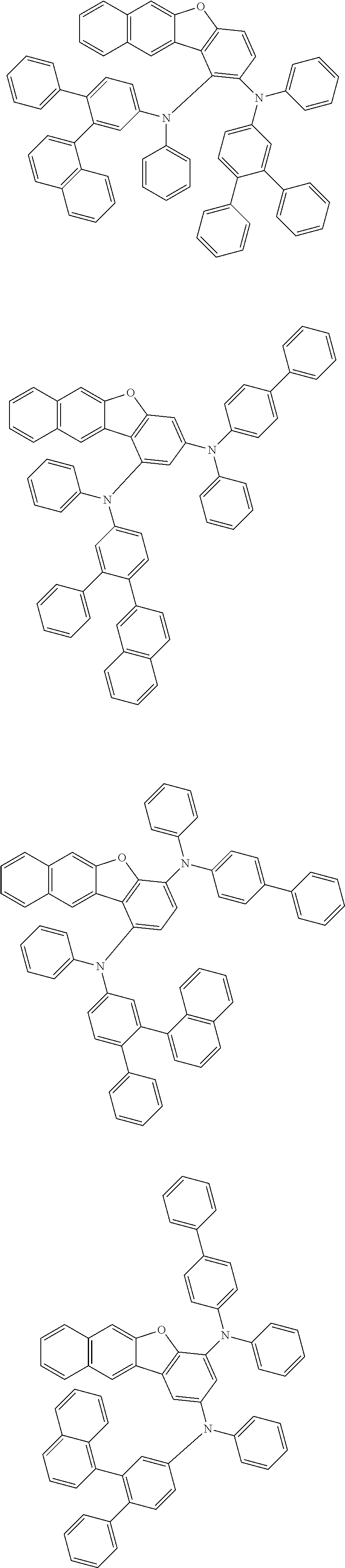 [SMILES: c1ccc(-c2ccc(N(c3ccccc3)c3ccc4oc5cc6ccccc6cc5c4c3N(c3ccccc3)c3ccc(-c4ccccc4)c(-c4cccc5ccccc45)c3)cc2-c2ccccc2)cc1 ; c1ccc(-c2ccc(N(c3ccccc3)c3cc(N(c4ccccc4)c4ccc(-c5ccc6ccccc6c5)c(-c5ccccc5)c4)c4c(c3)oc3cc5ccccc5cc34)cc2)cc1 ; c1ccc(-c2ccc(N(c3ccccc3)c3ccc(N(c4ccccc4)c4ccc(-c5ccccc5)c(-c5cccc6ccccc56)c4)c4c3oc3cc5ccccc5cc34)cc2)cc1 ; c1ccc(-c2ccc(N(c3ccccc3)c3cc(N(c4ccccc4)c4ccc(-c5ccccc5)c(-c5cccc6ccccc56)c4)cc4c3oc3cc5ccccc5cc34)cc2)cc1]

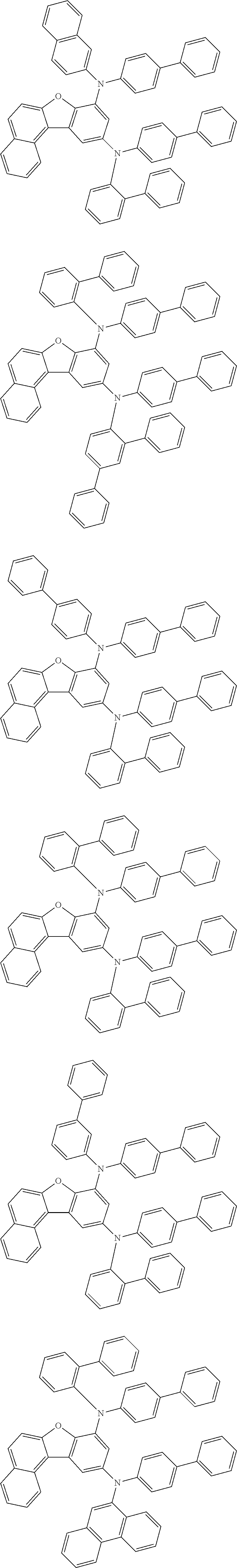

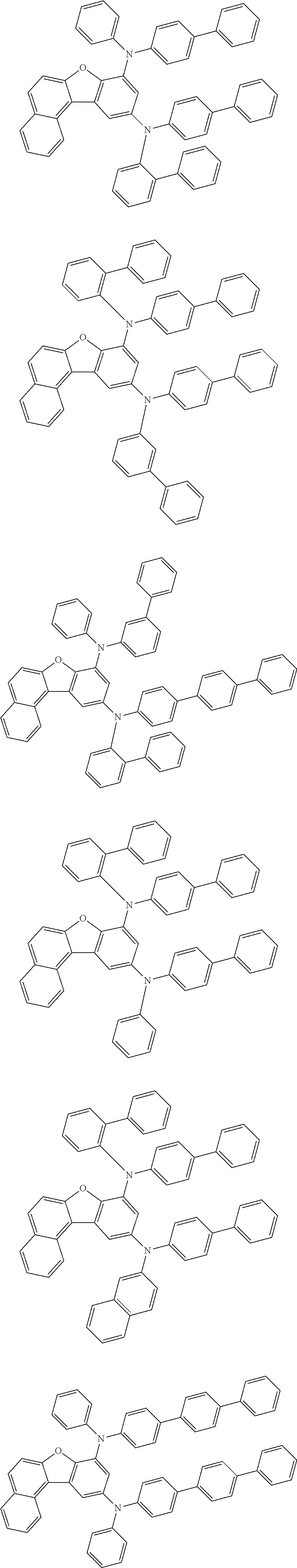

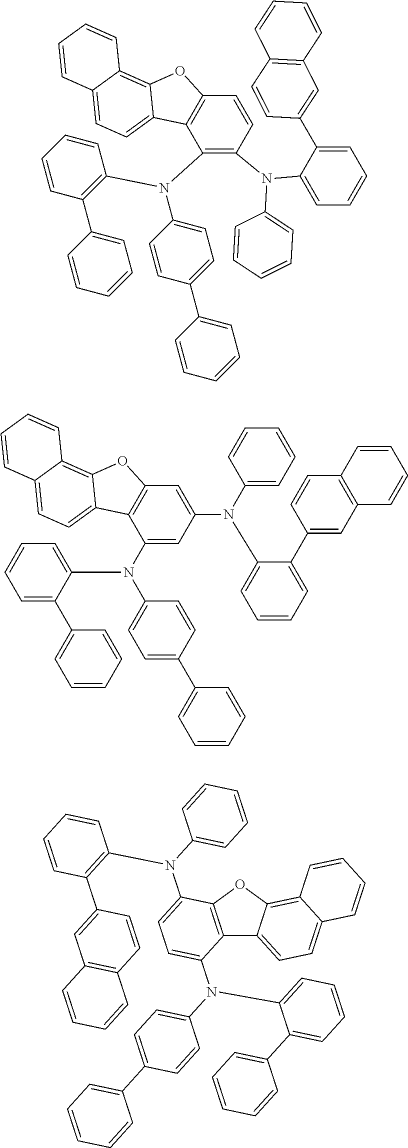

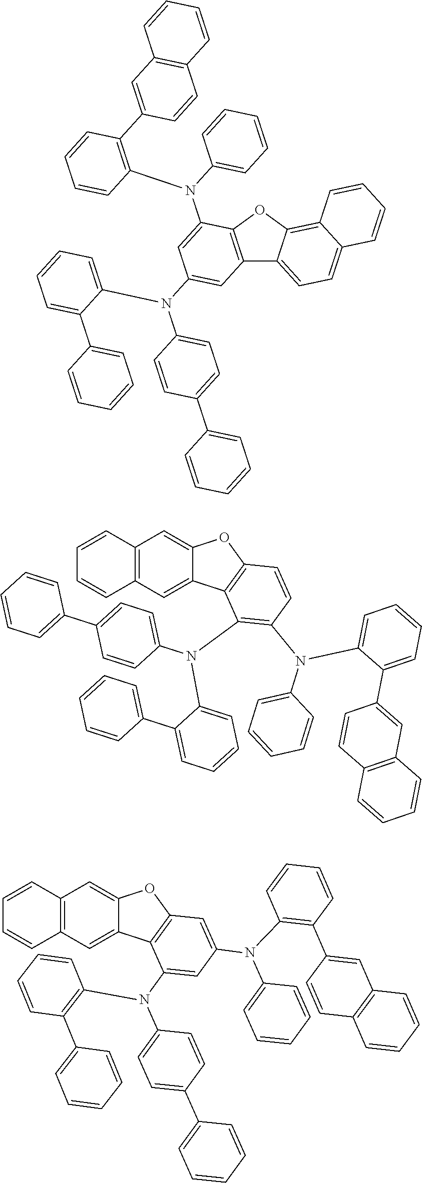

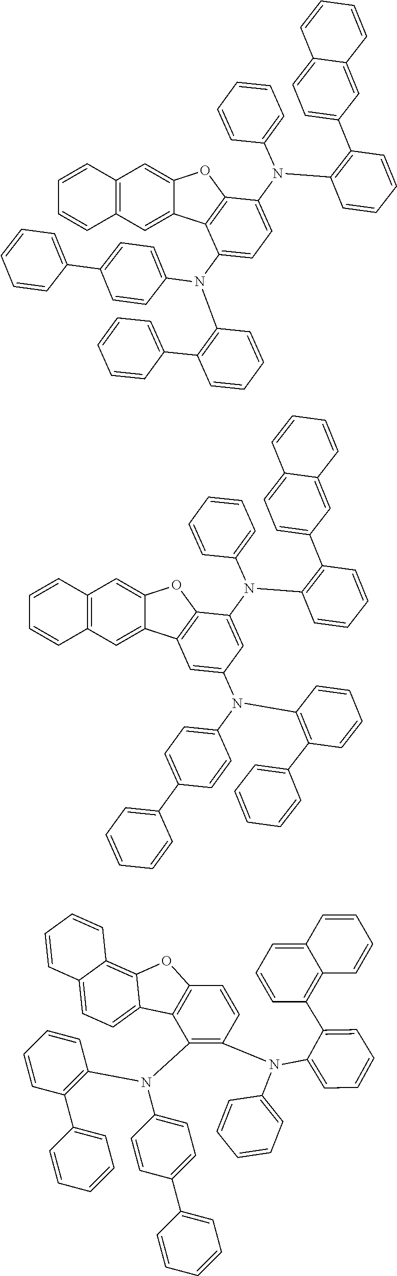 [SMILES: c1ccc(-c2ccc(N(c3ccccc3-c3ccccc3)c3ccc(N(c4ccccc4)c4ccccc4-c4ccc5ccccc5c4)c4oc5cc6ccccc6cc5c34)cc2)cc1 ; c1ccc(-c2ccc(N(c3cc(N(c4ccccc4)c4ccccc4-c4ccc5ccccc5c4)c4oc5cc6ccccc6cc5c4c3)c3ccccc3-c3ccccc3)cc2)cc1 ; c1ccc(-c2ccc(N(c3ccccc3-c3ccccc3)c3c(N(c4ccccc4)c4ccccc4-c4cccc5ccccc45)ccc4oc5c6ccccc6ccc5c34)cc2)cc1]

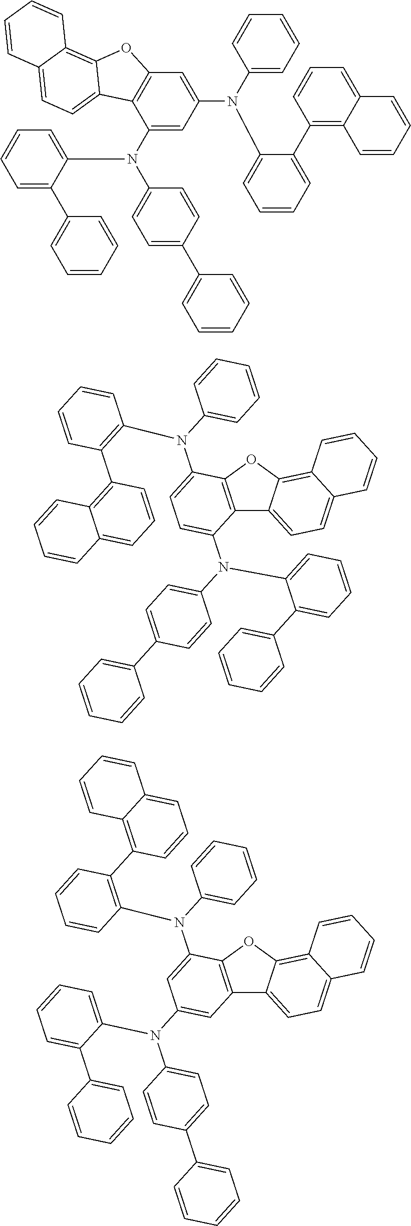

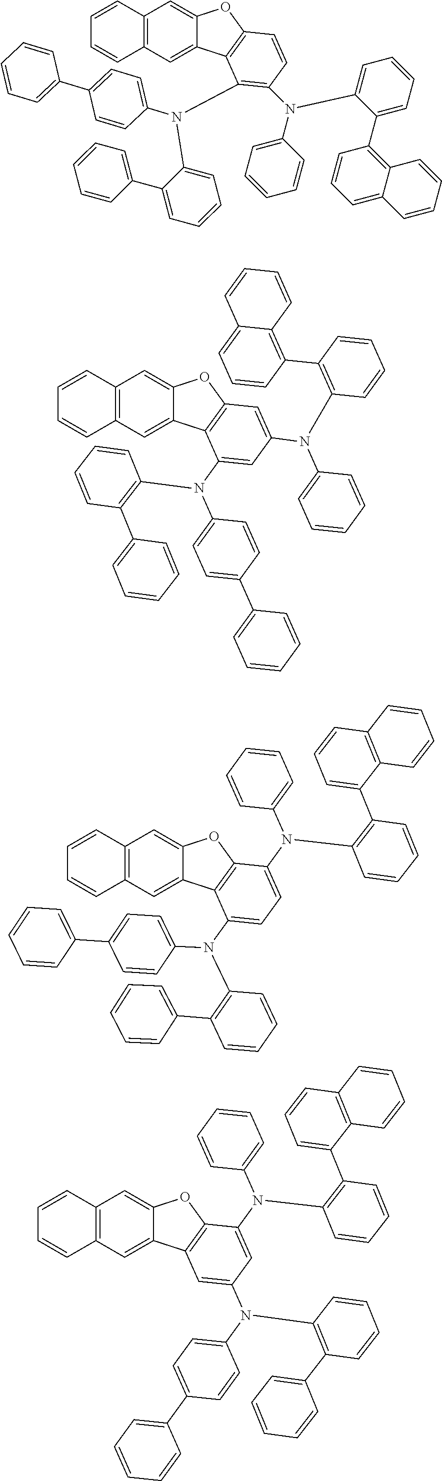 [SMILES: c1ccc(-c2ccc(N(c3ccccc3-c3ccccc3)c3c(N(c4ccccc4)c4ccccc4-c4cccc5ccccc45)ccc4oc5cc6ccccc6cc5c34)cc2)cc1 ; c1ccc(-c2ccc(N(c3ccccc3-c3ccccc3)c3cc(N(c4ccccc4)c4ccccc4-c4cccc5ccccc45)cc4oc5cc6ccccc6cc5c34)cc2)cc1 ; c1ccc(-c2ccc(N(c3ccccc3-c3ccccc3)c3ccc(N(c4ccccc4)c4ccccc4-c4cccc5ccccc45)c4oc5cc6ccccc6cc5c34)cc2)cc1 ; c1ccc(-c2ccc(N(c3cc(N(c4ccccc4)c4ccccc4-c4cccc5ccccc45)c4oc5cc6ccccc6cc5c4c3)c3ccccc3-c3ccccc3)cc2)cc1]

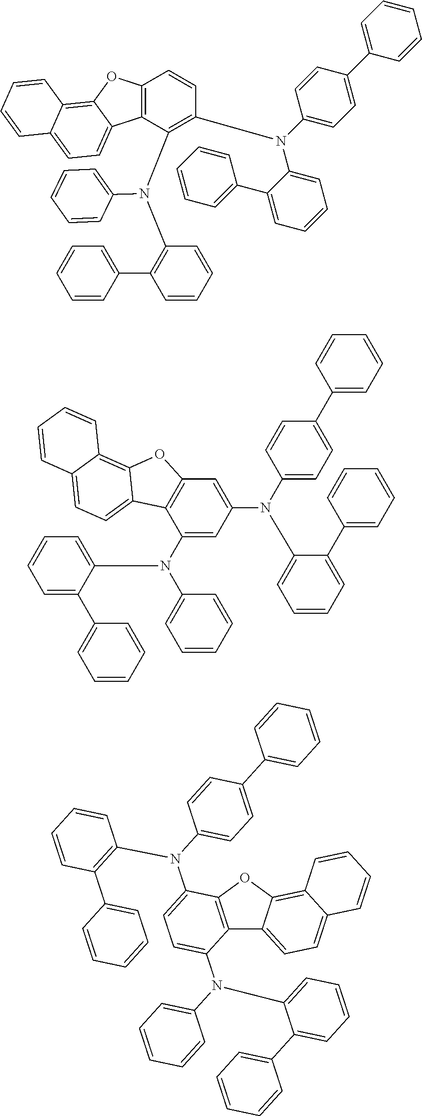 [SMILES: c1ccc(-c2ccc(N(c3ccccc3-c3ccccc3)c3ccc4oc5c6ccccc6ccc5c4c3N(c3ccccc3)c3ccccc3-c3ccccc3)cc2)cc1 ; c1ccc(-c2ccc(N(c3cc(N(c4ccccc4)c4ccccc4-c4ccccc4)c4c(c3)oc3c5ccccc5ccc34)c3ccccc3-c3ccccc3)cc2)cc1 ; c1ccc(-c2ccc(N(c3ccccc3-c3ccccc3)c3ccc(N(c4ccccc4)c4ccccc4-c4ccccc4)c4c3oc3c5ccccc5ccc34)cc2)cc1]

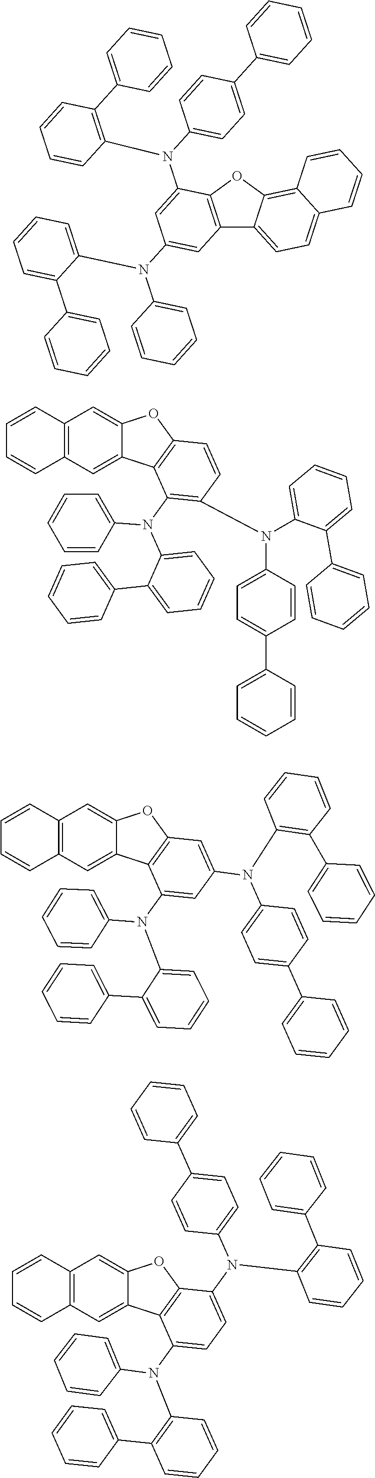

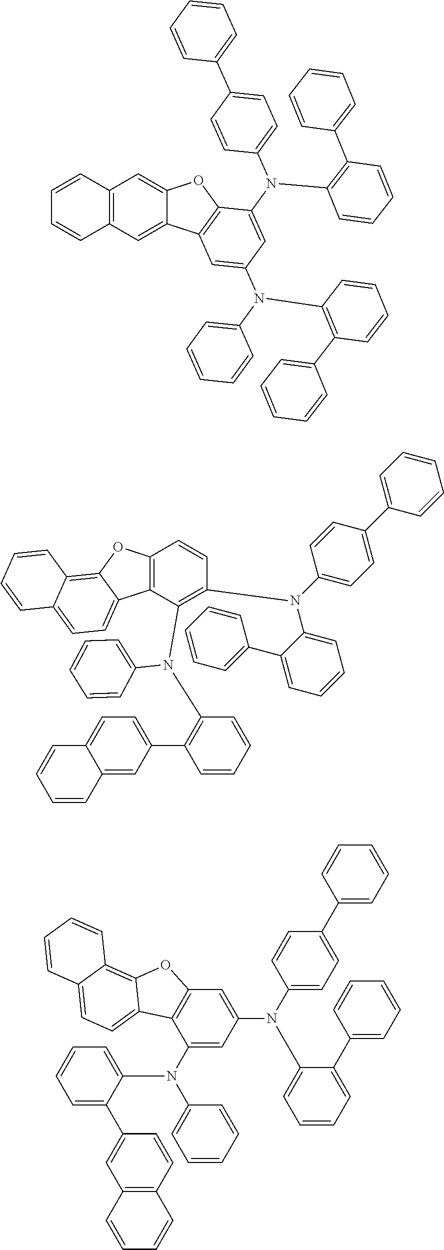 [SMILES: c1ccc(-c2ccc(N(c3ccccc3-c3ccccc3)c3cc(N(c4ccccc4)c4ccccc4-c4ccccc4)cc4c3oc3cc5ccccc5cc34)cc2)cc1 ; c1ccc(-c2ccc(N(c3ccccc3-c3ccccc3)c3ccc4oc5c6ccccc6ccc5c4c3N(c3ccccc3)c3ccccc3-c3ccc4ccccc4c3)cc2)cc1 ; c1ccc(-c2ccc(N(c3cc(N(c4ccccc4)c4ccccc4-c4ccc5ccccc5c4)c4c(c3)oc3c5ccccc5ccc34)c3ccccc3-c3ccccc3)cc2)cc1]

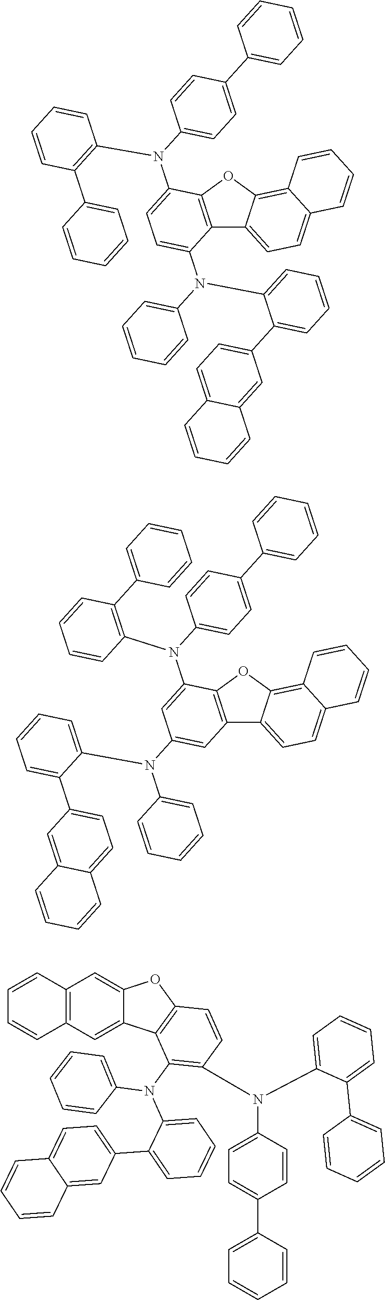 [SMILES: c1ccc(-c2ccc(N(c3ccccc3-c3ccccc3)c3ccc(N(c4ccccc4)c4ccccc4-c4ccc5ccccc5c4)c4c3oc3c5ccccc5ccc34)cc2)cc1 ; c1ccc(-c2ccc(N(c3ccccc3-c3ccccc3)c3cc(N(c4ccccc4)c4ccccc4-c4ccc5ccccc5c4)cc4c3oc3c5ccccc5ccc43)cc2)cc1 ; c1ccc(-c2ccc(N(c3ccccc3-c3ccccc3)c3ccc4oc5cc6ccccc6cc5c4c3N(c3ccccc3)c3ccccc3-c3ccc4ccccc4c3)cc2)cc1]

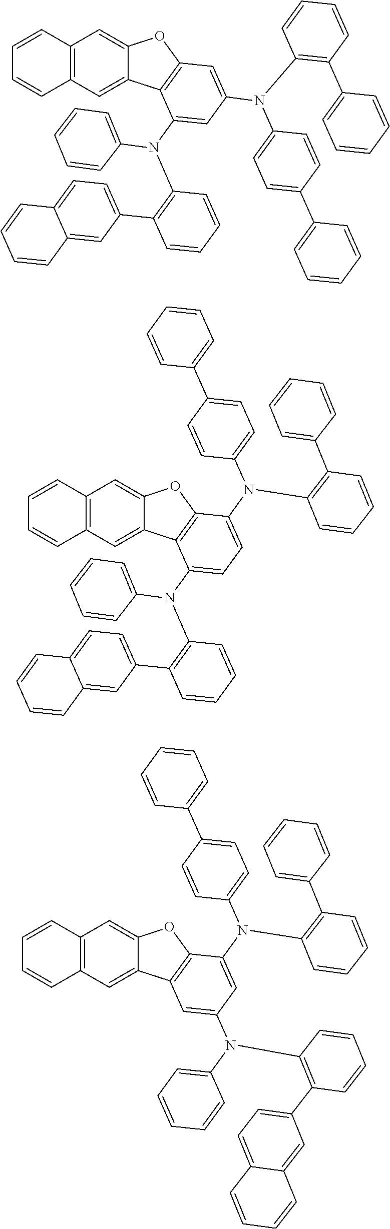 [SMILES: c1ccc(-c2ccc(N(c3cc(N(c4ccccc4)c4ccccc4-c4ccc5ccccc5c4)c4c(c3)oc3cc5ccccc5cc34)c3ccccc3-c3ccccc3)cc2)cc1 ; c1ccc(-c2ccc(N(c3ccccc3-c3ccccc3)c3ccc(N(c4ccccc4)c4ccccc4-c4ccc5ccccc5c4)c4c3oc3cc5ccccc5cc34)cc2)cc1 ; c1ccc(-c2ccc(N(c3ccccc3-c3ccccc3)c3cc(N(c4ccccc4)c4ccccc4-c4ccc5ccccc5c4)cc4c3oc3cc5ccccc5cc34)cc2)cc1]

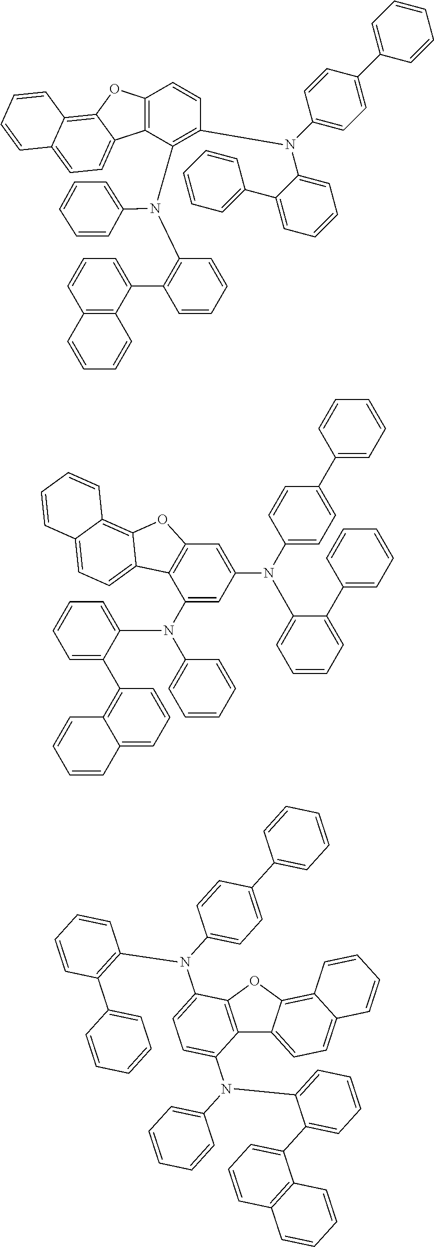 [SMILES: c1ccc(-c2ccc(N(c3ccccc3-c3ccccc3)c3ccc4oc5c6ccccc6ccc5c4c3N(c3ccccc3)c3ccccc3-c3cccc4ccccc34)cc2)cc1 ; c1ccc(-c2ccc(N(c3cc(N(c4ccccc4)c4ccccc4-c4cccc5ccccc45)c4c(c3)oc3c5ccccc5ccc34)c3ccccc3-c3ccccc3)cc2)cc1 ; c1ccc(-c2ccc(N(c3ccccc3-c3ccccc3)c3ccc(N(c4ccccc4)c4ccccc4-c4cccc5ccccc45)c4c3oc3c5ccccc5ccc34)cc2)cc1]

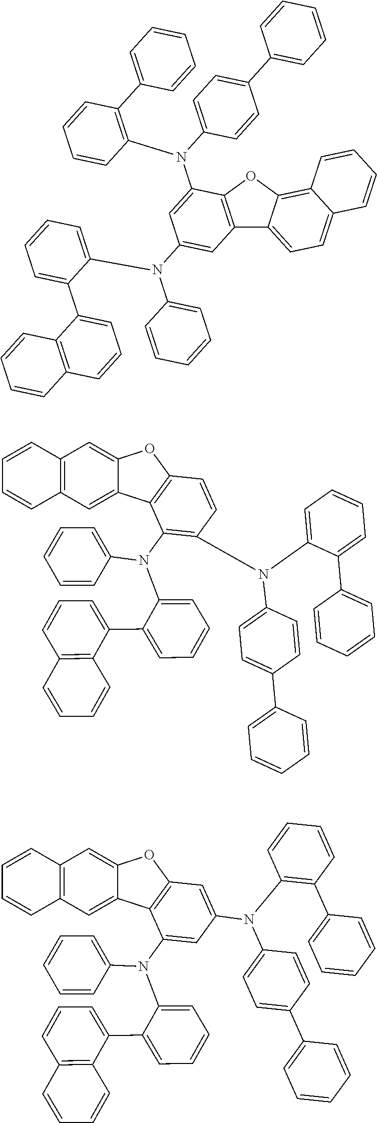 [SMILES: c1ccc(-c2ccc(N(c3ccccc3-c3ccccc3)c3cc(N(c4ccccc4)c4ccccc4-c4cccc5ccccc45)cc4c3oc3c5ccccc5ccc43)cc2)cc1 ; c1ccc(-c2ccc(N(c3ccccc3-c3ccccc3)c3ccc4oc5cc6ccccc6cc5c4c3N(c3ccccc3)c3ccccc3-c3cccc4ccccc34)cc2)cc1 ; c1ccc(-c2ccc(N(c3cc(N(c4ccccc4)c4ccccc4-c4cccc5ccccc45)c4c(c3)oc3cc5ccccc5cc34)c3ccccc3-c3ccccc3)cc2)cc1]

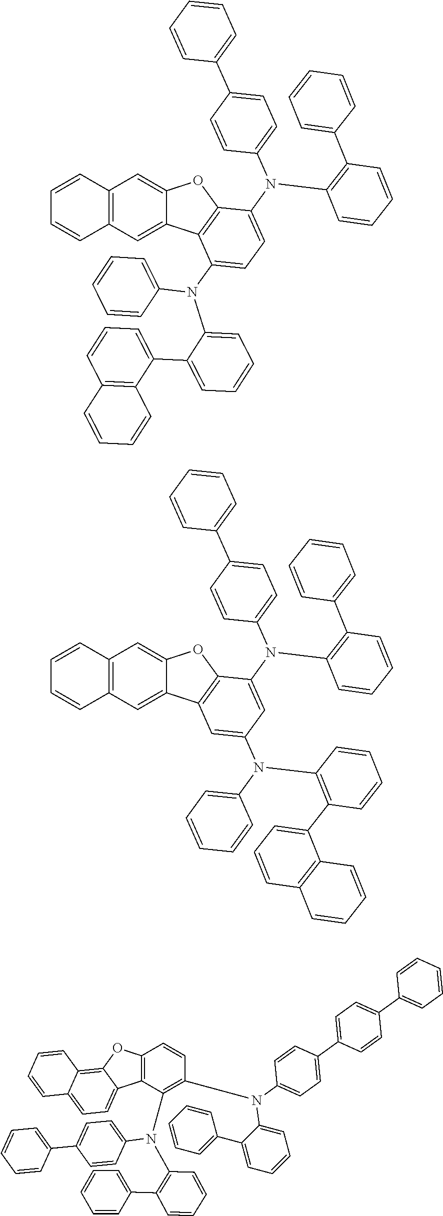 [SMILES: c1ccc(-c2ccc(N(c3ccccc3-c3ccccc3)c3ccc(N(c4ccccc4)c4ccccc4-c4cccc5ccccc45)c4c3oc3cc5ccccc5cc34)cc2)cc1 ; c1ccc(-c2ccc(N(c3ccccc3-c3ccccc3)c3cc(N(c4ccccc4)c4ccccc4-c4cccc5ccccc45)cc4c3oc3cc5ccccc5cc34)cc2)cc1 ; c1ccc(-c2ccc(-c3ccc(N(c4ccccc4-c4ccccc4)c4ccc5oc6c7ccccc7ccc6c5c4N(c4ccc(-c5ccccc5)cc4)c4ccccc4-c4ccccc4)cc3)cc2)cc1]

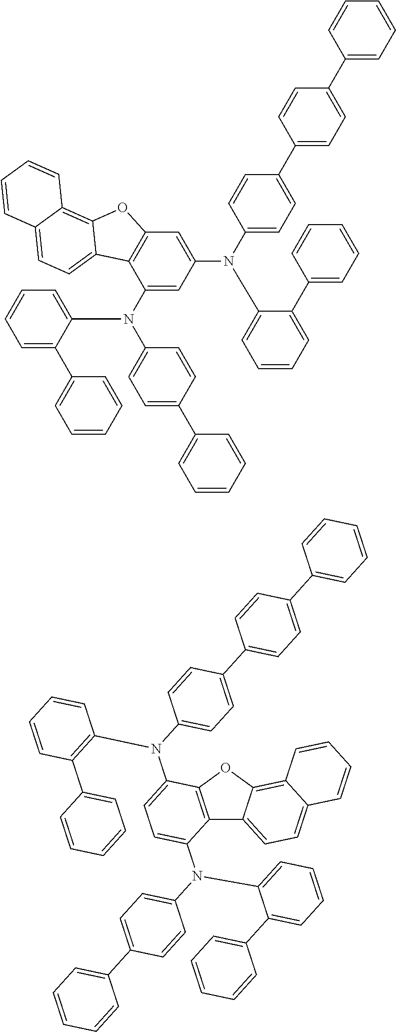 [SMILES: c1ccc(-c2ccc(-c3ccc(N(c4cc(N(c5ccc(-c6ccccc6)cc5)c5ccccc5-c5ccccc5)c5c(c4)oc4c6ccccc6ccc45)c4ccccc4-c4ccccc4)cc3)cc2)cc1 ; c1ccc(-c2ccc(-c3ccc(N(c4ccccc4-c4ccccc4)c4ccc(N(c5ccc(-c6ccccc6)cc5)c5ccccc5-c5ccccc5)c5c4oc4c6ccccc6ccc45)cc3)cc2)cc1]

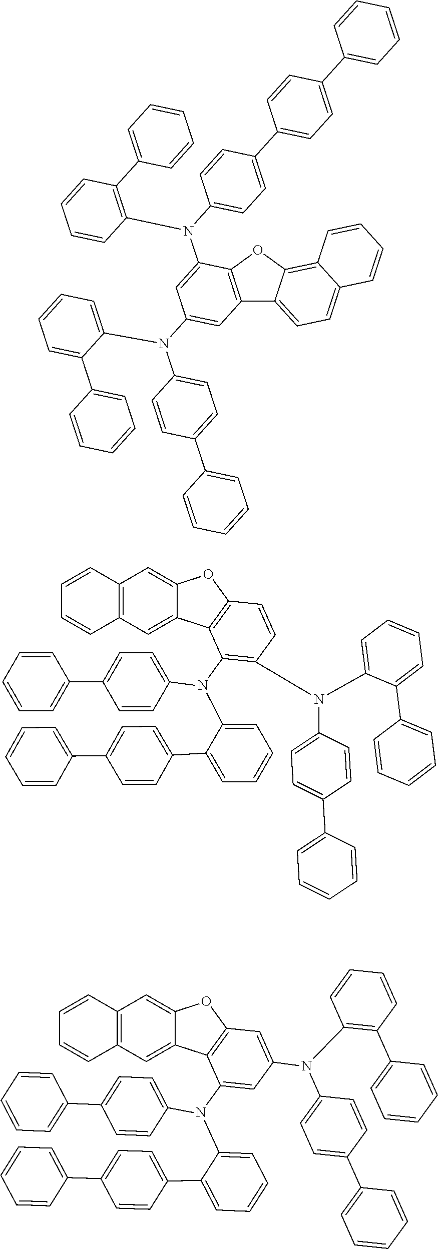

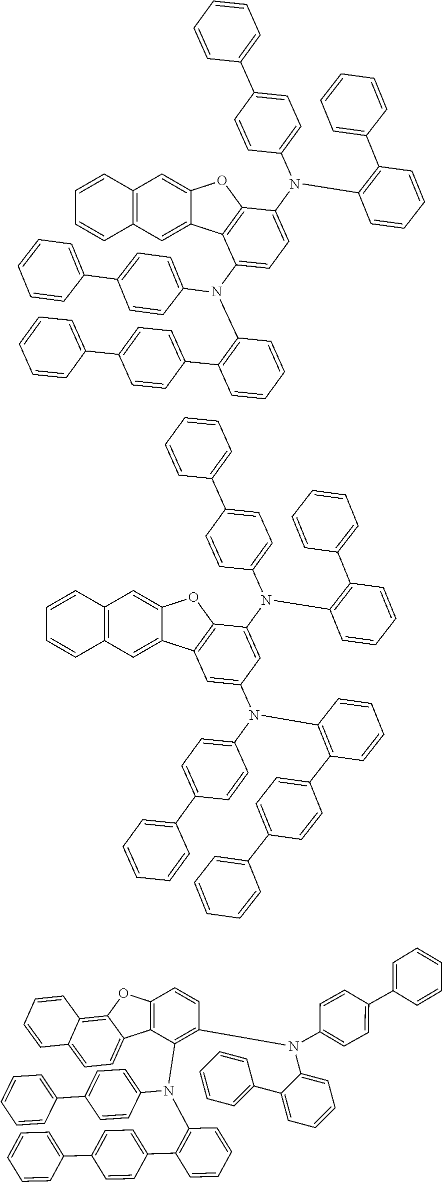

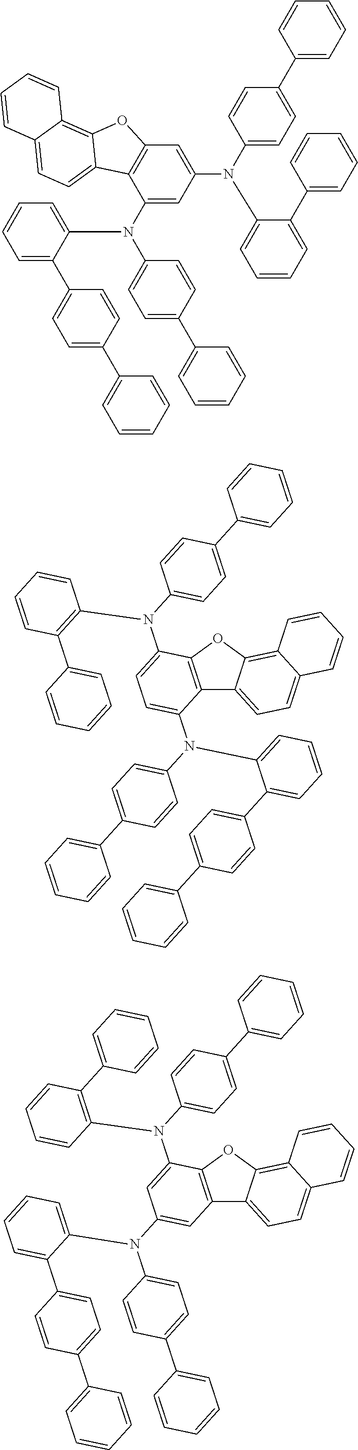 [SMILES: c1ccc(-c2ccc(-c3ccccc3N(c3ccc(-c4ccccc4)cc3)c3cc(N(c4ccc(-c5ccccc5)cc4)c4ccccc4-c4ccccc4)cc4oc5c6ccccc6ccc5c34)cc2)cc1 ; c1ccc(-c2ccc(-c3ccccc3N(c3ccc(-c4ccccc4)cc3)c3ccc(N(c4ccc(-c5ccccc5)cc4)c4ccccc4-c4ccccc4)c4oc5c6ccccc6ccc5c34)cc2)cc1 ; c1ccc(-c2ccc(-c3ccccc3N(c3ccc(-c4ccccc4)cc3)c3cc(N(c4ccc(-c5ccccc5)cc4)c4ccccc4-c4ccccc4)c4oc5c6ccccc6ccc5c4c3)cc2)cc1]

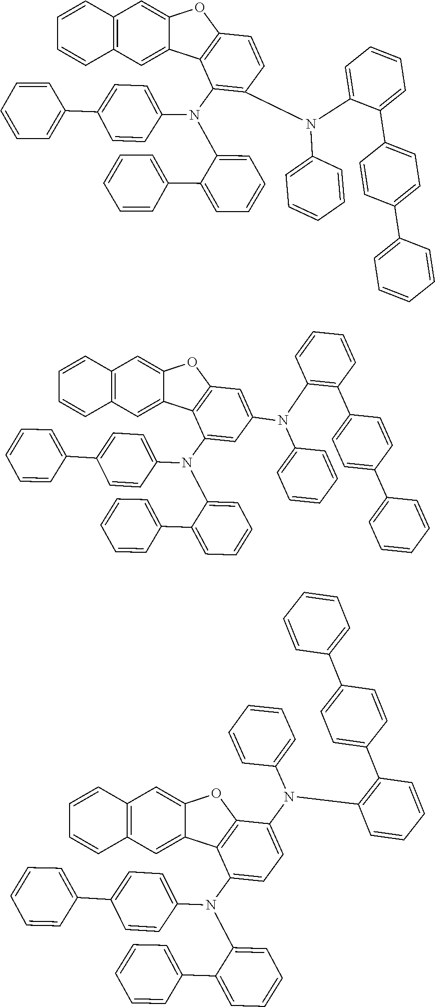 [SMILES: c1ccc(-c2ccc(-c3ccccc3N(c3ccccc3)c3ccc4oc5cc6ccccc6cc5c4c3N(c3ccc(-c4ccccc4)cc3)c3ccccc3-c3ccccc3)cc2)cc1 ; c1ccc(-c2ccc(-c3ccccc3N(c3ccccc3)c3cc(N(c4ccc(-c5ccccc5)cc4)c4ccccc4-c4ccccc4)c4c(c3)oc3cc5ccccc5cc34)cc2)cc1 ; c1ccc(-c2ccc(-c3ccccc3N(c3ccccc3)c3ccc(N(c4ccc(-c5ccccc5)cc4)c4ccccc4-c4ccccc4)c4c3oc3cc5ccccc5cc34)cc2)cc1]

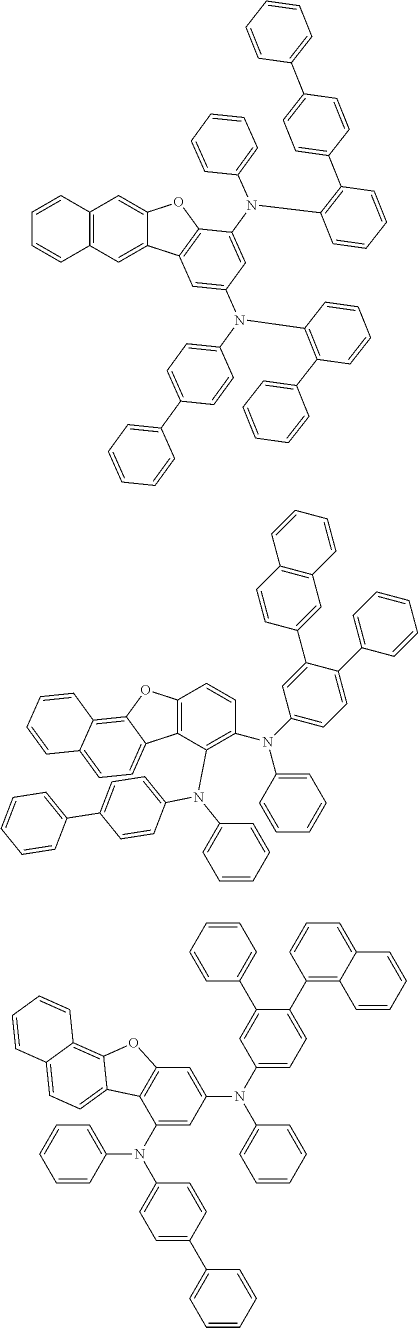

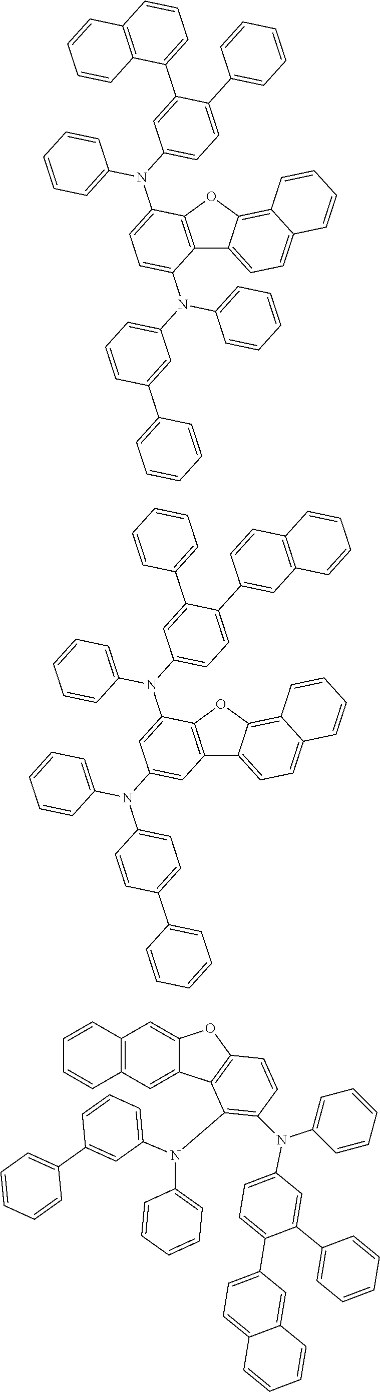

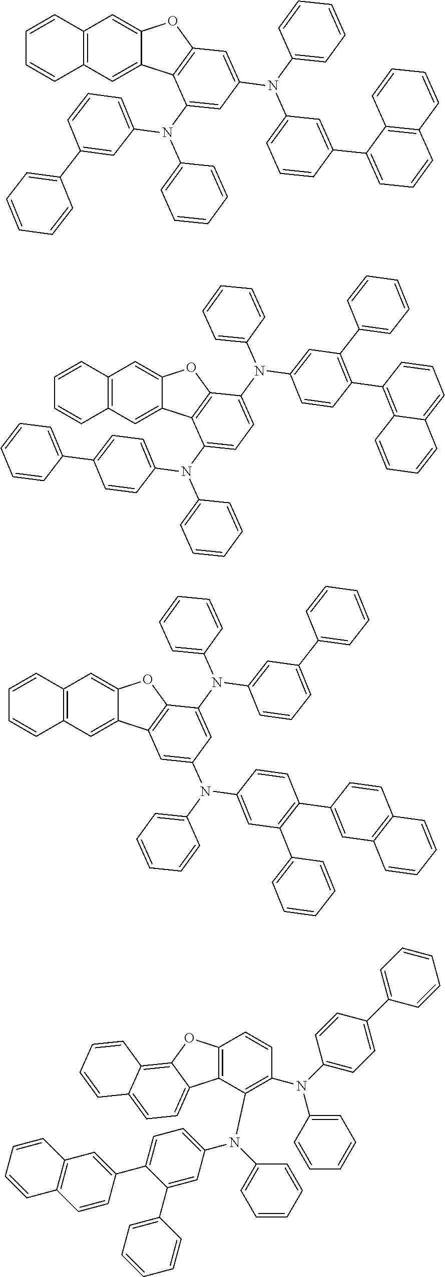 [SMILES: c1ccc(-c2cccc(N(c3ccccc3)c3cc(N(c4ccccc4)c4cccc(-c5cccc6ccccc56)c4)cc4oc5cc6ccccc6cc5c34)c2)cc1 ; c1ccc(-c2ccc(N(c3ccccc3)c3ccc(N(c4ccccc4)c4ccc(-c5cccc6ccccc56)c(-c5ccccc5)c4)c4oc5cc6ccccc6cc5c34)cc2)cc1 ; c1ccc(-c2cccc(N(c3ccccc3)c3cc(N(c4ccccc4)c4ccc(-c5ccc6ccccc6c5)c(-c5ccccc5)c4)cc4c3oc3cc5ccccc5cc34)c2)cc1 ; c1ccc(-c2ccc(N(c3ccccc3)c3ccc4oc5c6ccccc6ccc5c4c3N(c3ccccc3)c3ccc(-c4ccc5ccccc5c4)c(-c4ccccc4)c3)cc2)cc1]

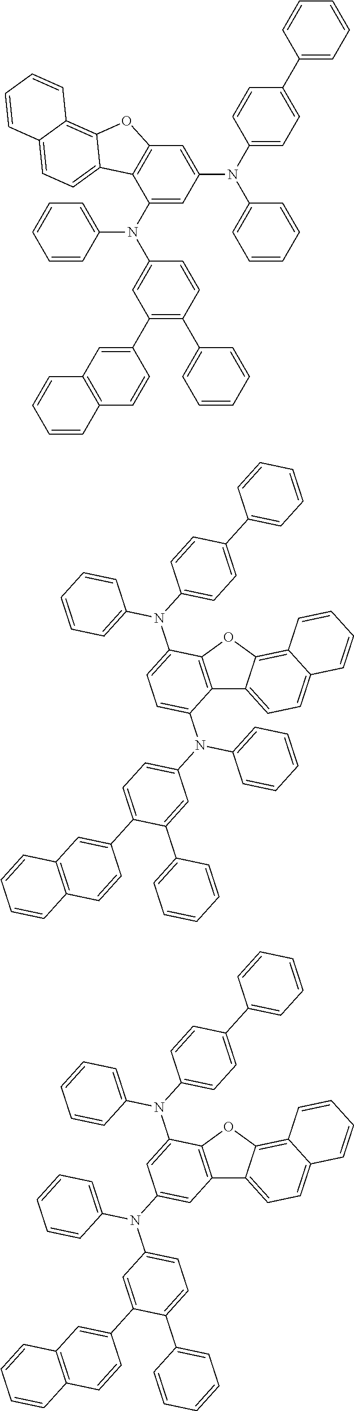 [SMILES: c1ccc(-c2ccc(N(c3ccccc3)c3cc(N(c4ccccc4)c4ccc(-c5ccccc5)c(-c5ccc6ccccc6c5)c4)c4c(c3)oc3c5ccccc5ccc34)cc2)cc1 ; c1ccc(-c2ccc(N(c3ccccc3)c3ccc(N(c4ccccc4)c4ccc(-c5ccc6ccccc6c5)c(-c5ccccc5)c4)c4c3oc3c5ccccc5ccc34)cc2)cc1 ; c1ccc(-c2ccc(N(c3ccccc3)c3cc(N(c4ccccc4)c4ccc(-c5ccccc5)c(-c5ccc6ccccc6c5)c4)cc4c3oc3c5ccccc5ccc43)cc2)cc1]

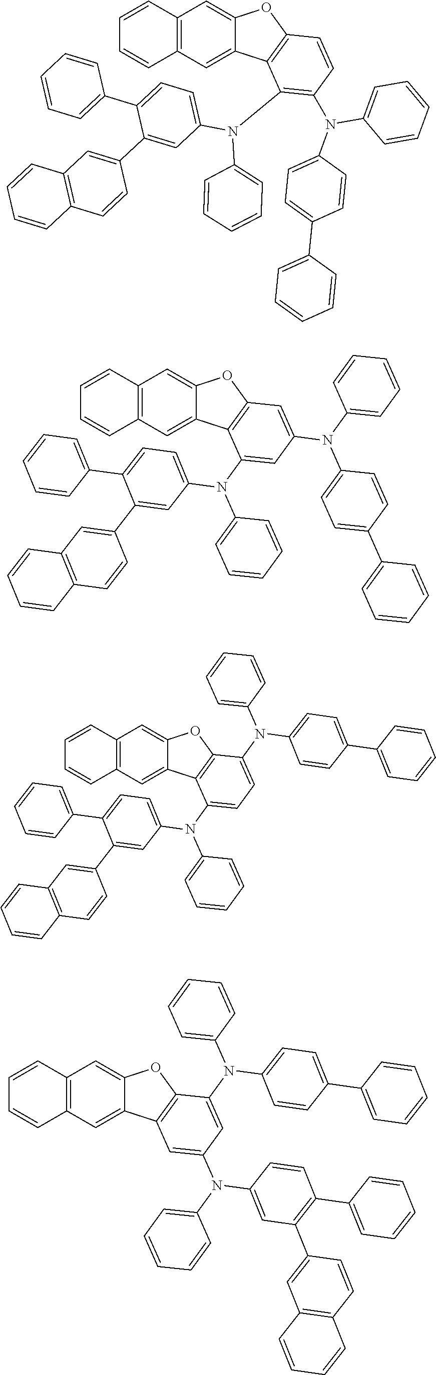 [SMILES: c1ccc(-c2ccc(N(c3ccccc3)c3ccc4oc5cc6ccccc6cc5c4c3N(c3ccccc3)c3ccc(-c4ccccc4)c(-c4ccc5ccccc5c4)c3)cc2)cc1 ; c1ccc(-c2ccc(N(c3ccccc3)c3cc(N(c4ccccc4)c4ccc(-c5ccccc5)c(-c5ccc6ccccc6c5)c4)c4c(c3)oc3cc5ccccc5cc34)cc2)cc1 ; c1ccc(-c2ccc(N(c3ccccc3)c3ccc(N(c4ccccc4)c4ccc(-c5ccccc5)c(-c5ccc6ccccc6c5)c4)c4c3oc3cc5ccccc5cc34)cc2)cc1 ; c1ccc(-c2ccc(N(c3ccccc3)c3cc(N(c4ccccc4)c4ccc(-c5ccccc5)c(-c5ccc6ccccc6c5)c4)cc4c3oc3cc5ccccc5cc34)cc2)cc1]

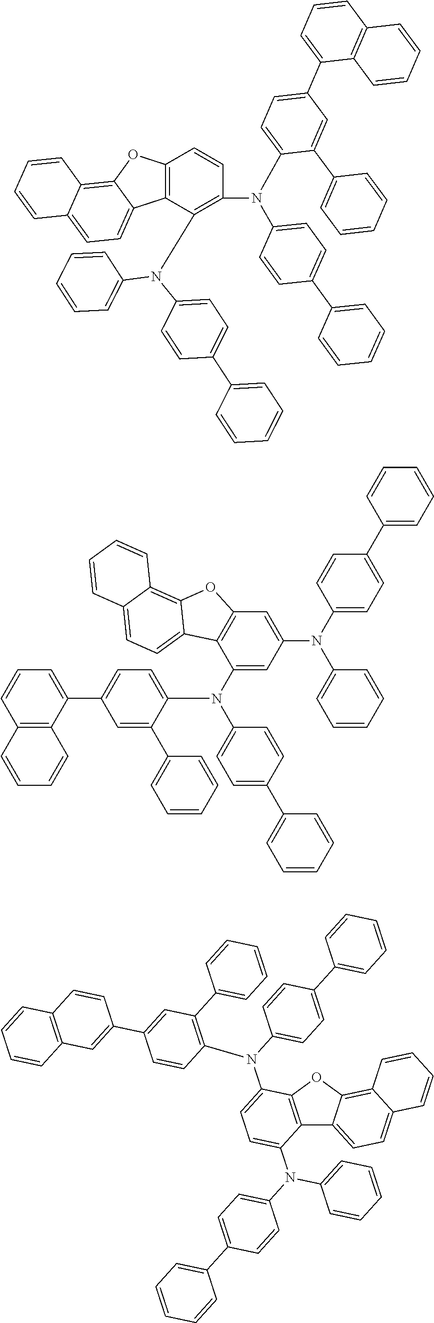 [SMILES: c1ccc(-c2ccc(N(c3ccc(-c4cccc5ccccc45)cc3-c3ccccc3)c3ccc4oc5c6ccccc6ccc5c4c3N(c3ccccc3)c3ccc(-c4ccccc4)cc3)cc2)cc1 ; c1ccc(-c2ccc(N(c3ccccc3)c3cc(N(c4ccc(-c5ccccc5)cc4)c4ccc(-c5cccc6ccccc56)cc4-c4ccccc4)c4c(c3)oc3c5ccccc5ccc34)cc2)cc1 ; c1ccc(-c2ccc(N(c3ccc(-c4ccc5ccccc5c4)cc3-c3ccccc3)c3ccc(N(c4ccccc4)c4ccc(-c5ccccc5)cc4)c4c3oc3c5ccccc5ccc34)cc2)cc1]

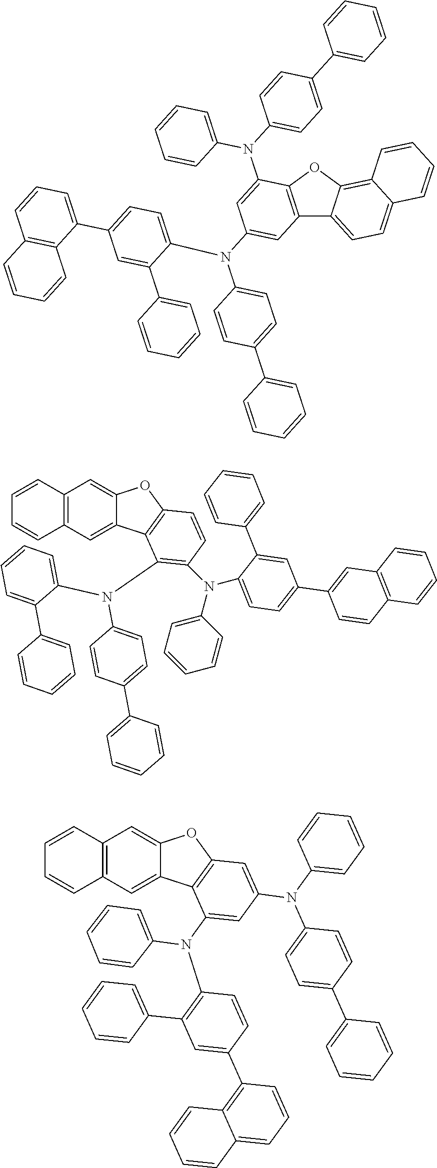

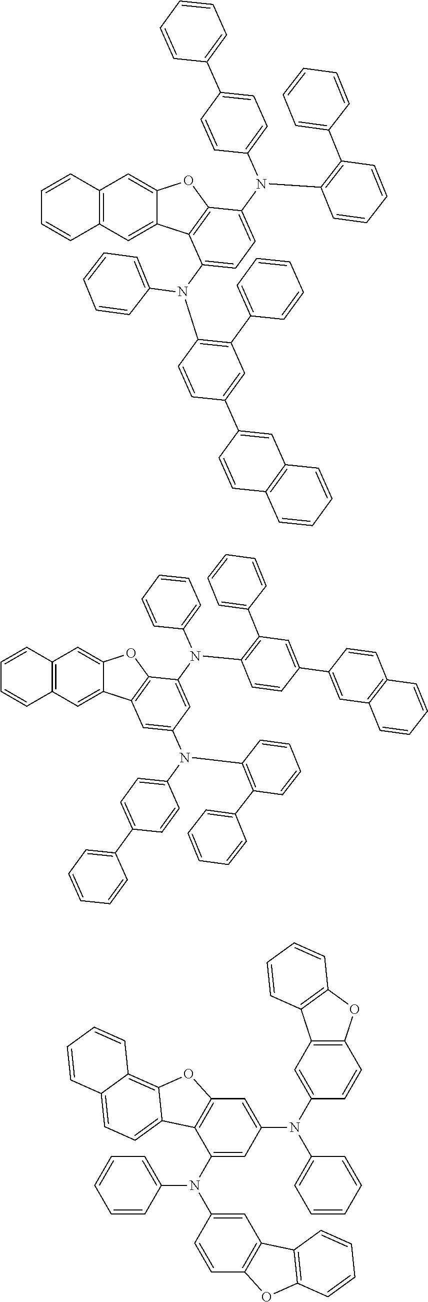

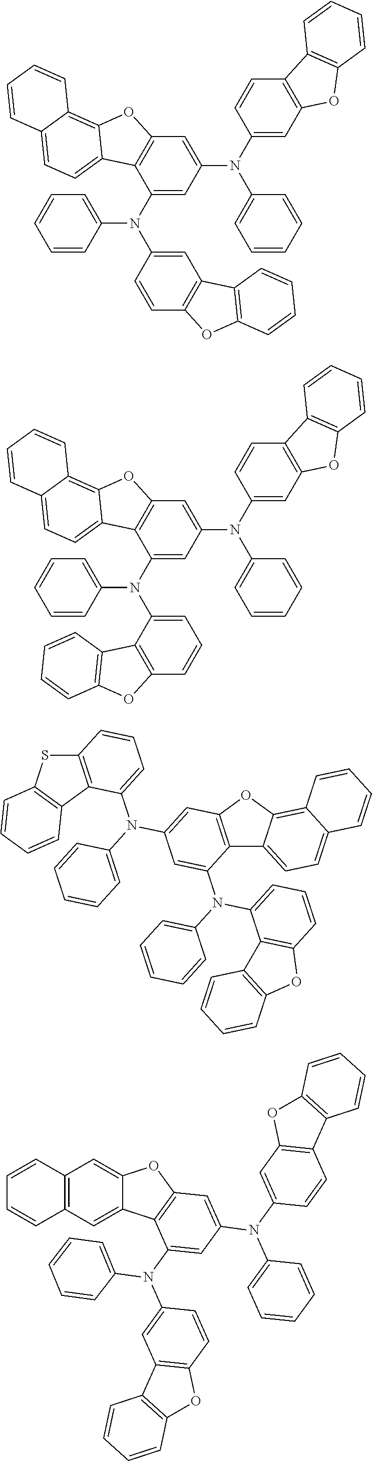

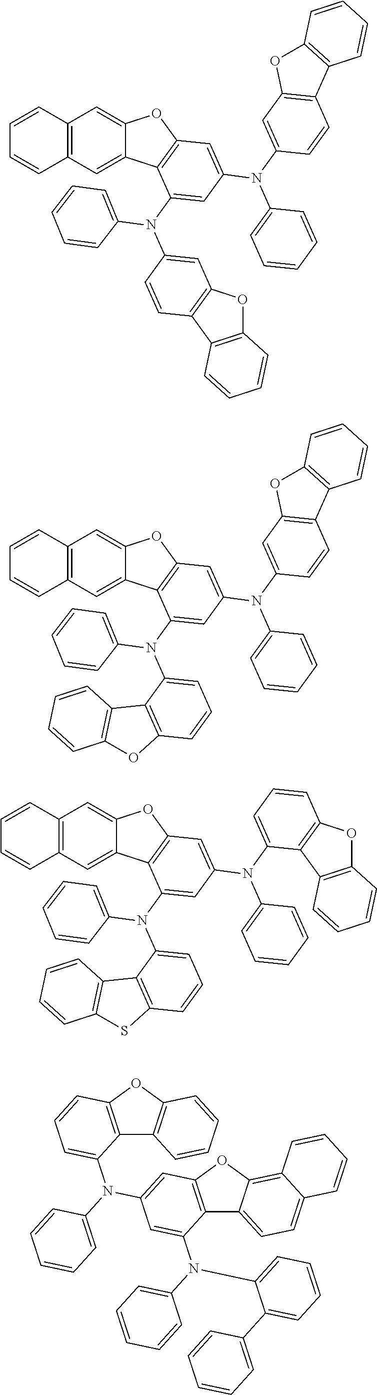 [SMILES: c1ccc(N(c2ccc3c(c2)oc2ccccc23)c2cc(N(c3ccccc3)c3ccc4c(c3)oc3ccccc34)c3c(c2)oc2cc4ccccc4cc23)cc1 ; c1ccc(N(c2ccc3c(c2)oc2ccccc23)c2cc(N(c3ccccc3)c3cccc4oc5ccccc5c34)c3c(c2)oc2cc4ccccc4cc23)cc1 ; c1ccc(N(c2cc(N(c3ccccc3)c3cccc4sc5ccccc5c34)c3c(c2)oc2cc4ccccc4cc23)c2cccc3oc4ccccc4c23)cc1 ; c1ccc(-c2ccccc2N(c2ccccc2)c2cc(N(c3ccccc3)c3cccc4oc5ccccc5c34)cc3oc4c5ccccc5ccc4c23)cc1]

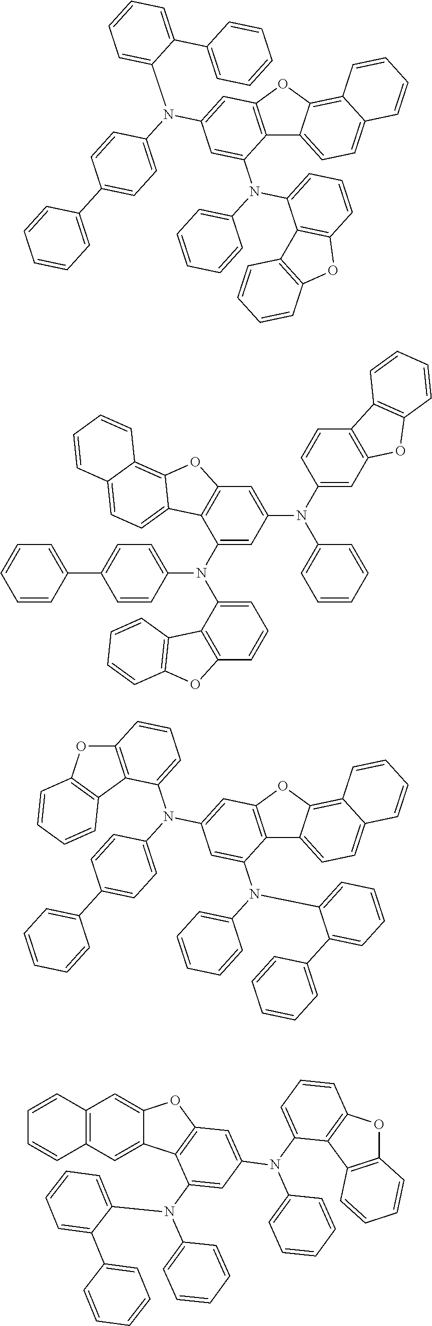

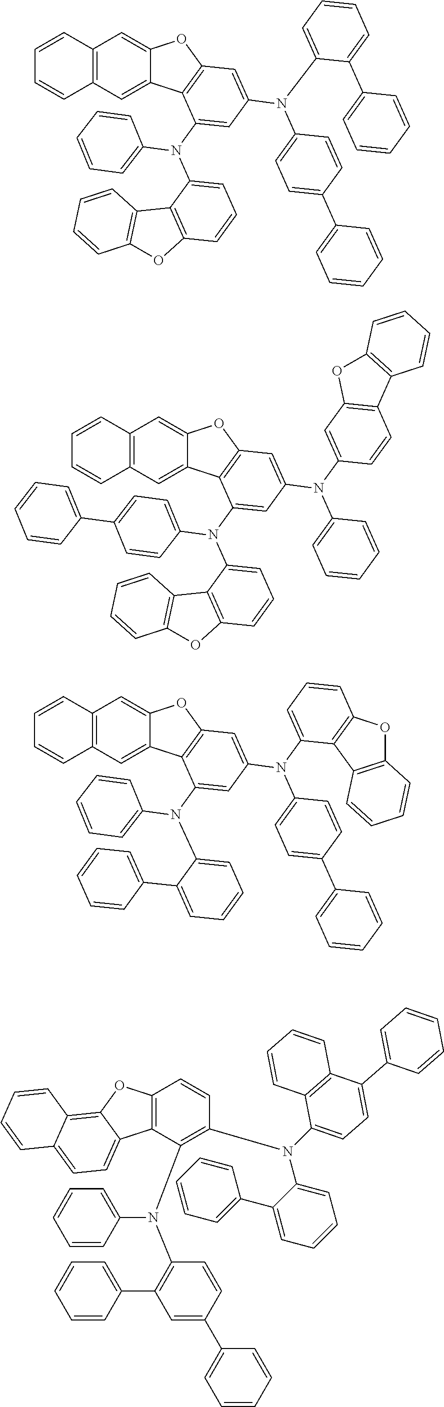

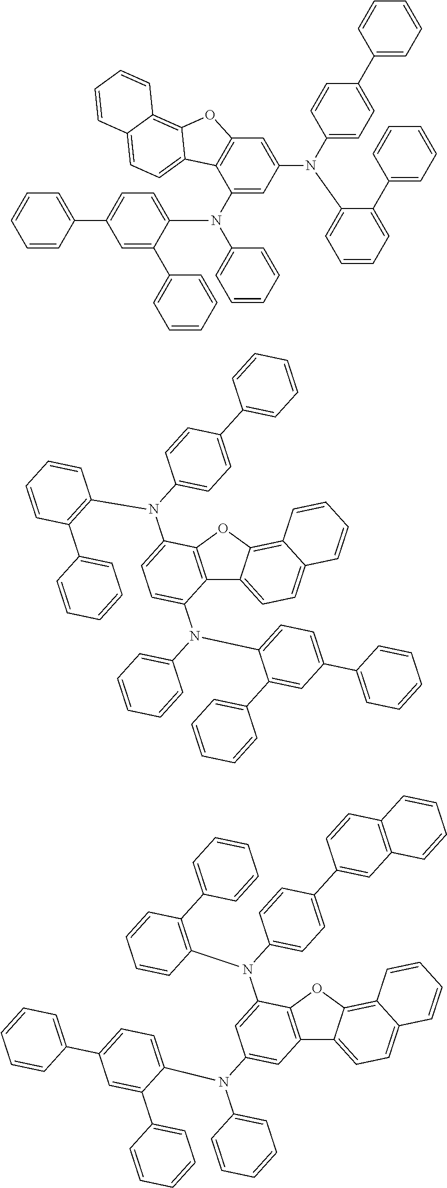 [SMILES: c1ccc(-c2ccc(N(c3cc(N(c4ccccc4)c4ccc(-c5ccccc5)cc4-c4ccccc4)c4c(c3)oc3c5ccccc5ccc34)c3ccccc3-c3ccccc3)cc2)cc1 ; c1ccc(-c2ccc(N(c3ccccc3-c3ccccc3)c3ccc(N(c4ccccc4)c4ccc(-c5ccccc5)cc4-c4ccccc4)c4c3oc3c5ccccc5ccc34)cc2)cc1 ; c1ccc(-c2ccc(N(c3ccccc3)c3cc(N(c4ccc(-c5ccc6ccccc6c5)cc4)c4ccccc4-c4ccccc4)c4oc5c6ccccc6ccc5c4c3)c(-c3ccccc3)c2)cc1]

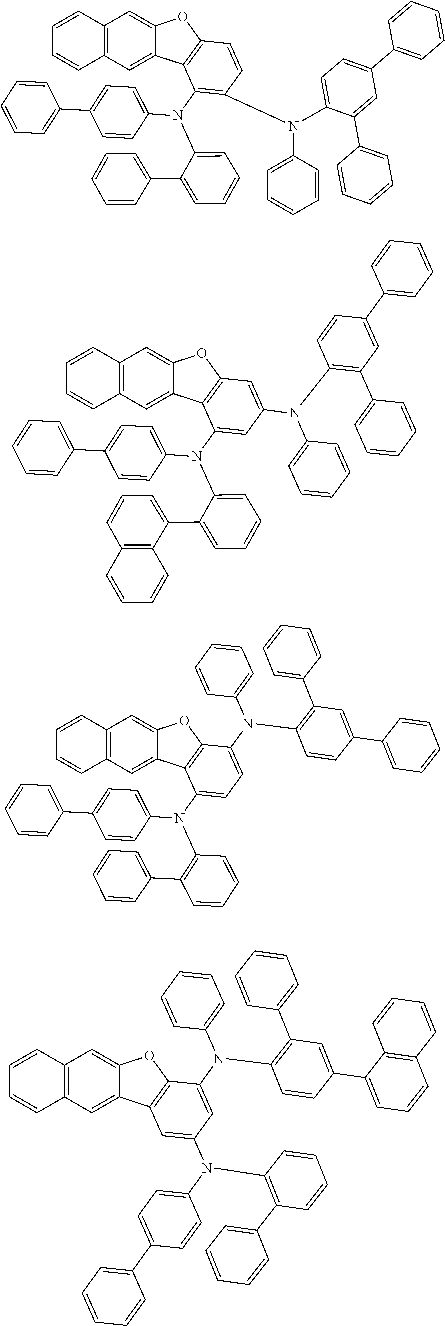

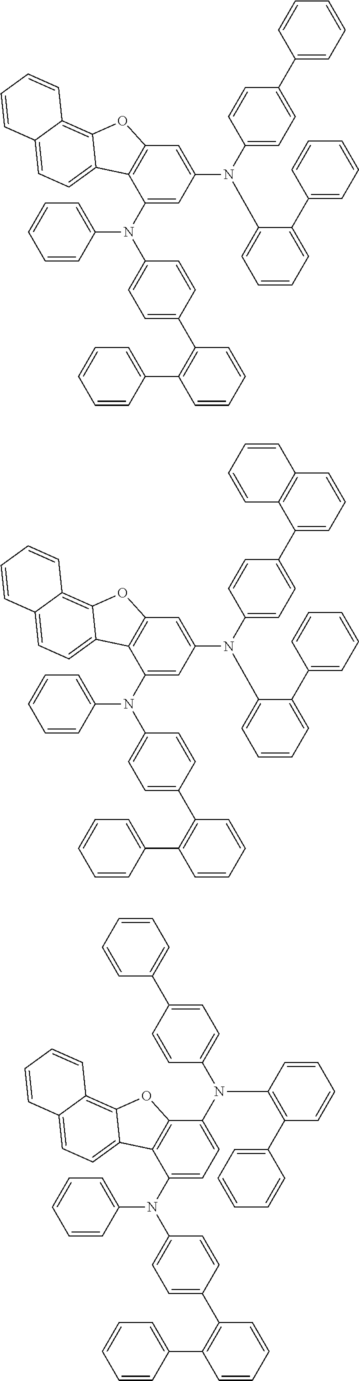

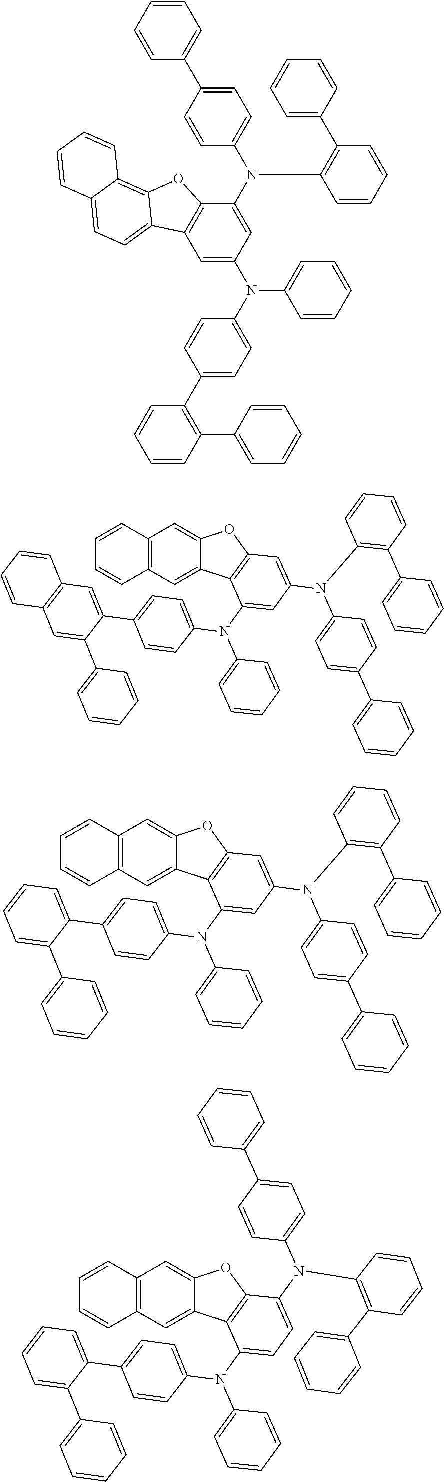

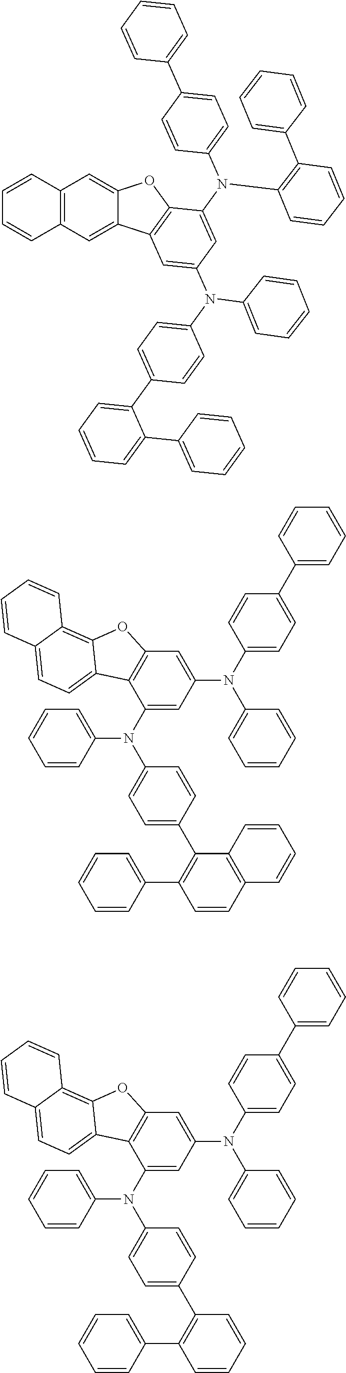 [SMILES: c1ccc(-c2ccc(N(c3ccccc3-c3ccccc3)c3cc(N(c4ccccc4)c4ccc(-c5ccccc5-c5ccccc5)cc4)cc4c3oc3cc5ccccc5cc34)cc2)cc1 ; c1ccc(-c2ccc(N(c3ccccc3)c3cc(N(c4ccccc4)c4ccc(-c5c(-c6ccccc6)ccc6ccccc56)cc4)c4c(c3)oc3c5ccccc5ccc34)cc2)cc1 ; c1ccc(-c2ccc(N(c3ccccc3)c3cc(N(c4ccccc4)c4ccc(-c5ccccc5-c5ccccc5)cc4)c4c(c3)oc3c5ccccc5ccc34)cc2)cc1]

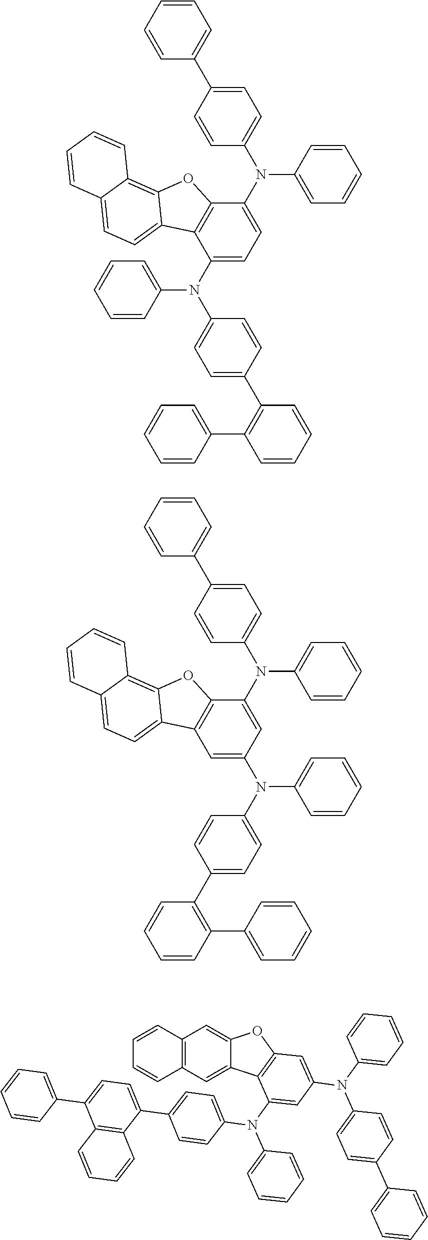

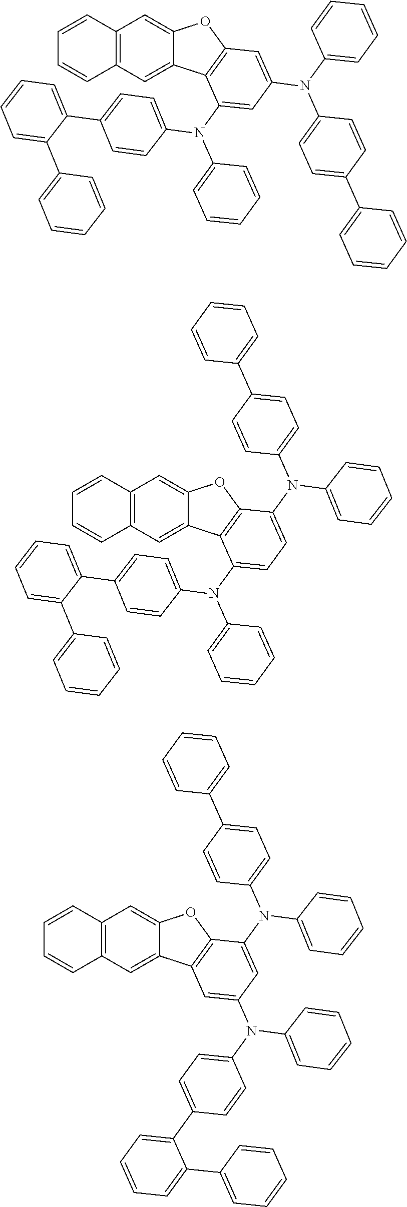 [SMILES: c1ccc(-c2ccc(N(c3ccccc3)c3cc(N(c4ccccc4)c4ccc(-c5ccccc5-c5ccccc5)cc4)c4c(c3)oc3cc5ccccc5cc34)cc2)cc1 ; c1ccc(-c2ccc(N(c3ccccc3)c3ccc(N(c4ccccc4)c4ccc(-c5ccccc5-c5ccccc5)cc4)c4c3oc3cc5ccccc5cc34)cc2)cc1 ; c1ccc(-c2ccc(N(c3ccccc3)c3cc(N(c4ccccc4)c4ccc(-c5ccccc5-c5ccccc5)cc4)cc4c3oc3cc5ccccc5cc34)cc2)cc1]

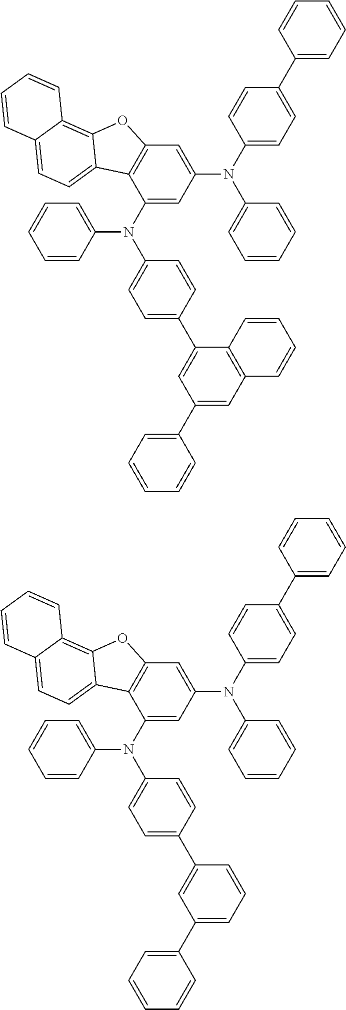 [SMILES: c1ccc(-c2ccc(N(c3ccccc3)c3cc(N(c4ccccc4)c4ccc(-c5cc(-c6ccccc6)cc6ccccc56)cc4)c4c(c3)oc3c5ccccc5ccc34)cc2)cc1 ; c1ccc(-c2ccc(N(c3ccccc3)c3cc(N(c4ccccc4)c4ccc(-c5cccc(-c6ccccc6)c5)cc4)c4c(c3)oc3c5ccccc5ccc34)cc2)cc1]

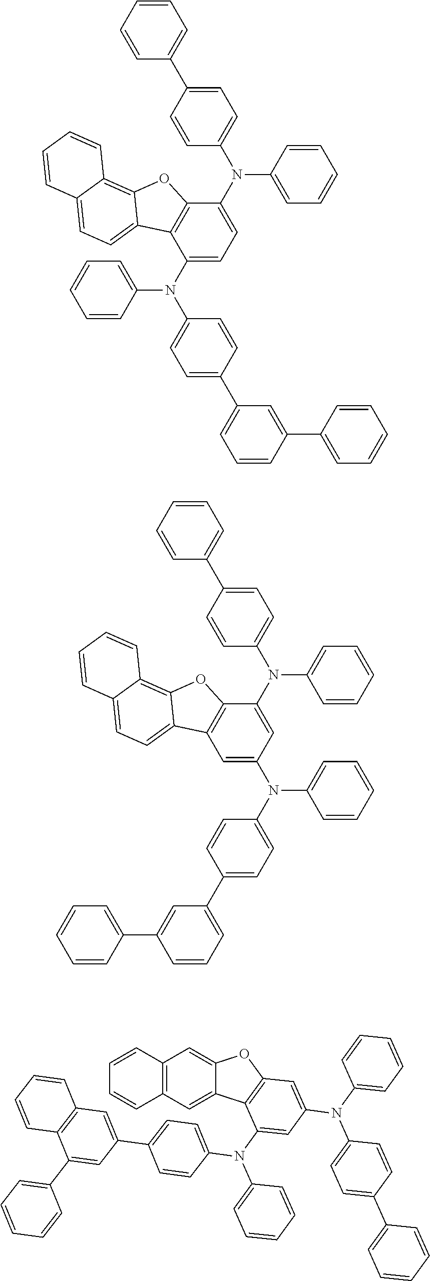 [SMILES: c1ccc(-c2ccc(N(c3ccccc3)c3ccc(N(c4ccccc4)c4ccc(-c5cccc(-c6ccccc6)c5)cc4)c4c3oc3c5ccccc5ccc34)cc2)cc1 ; c1ccc(-c2ccc(N(c3ccccc3)c3cc(N(c4ccccc4)c4ccc(-c5cccc(-c6ccccc6)c5)cc4)cc4c3oc3c5ccccc5ccc43)cc2)cc1 ; c1ccc(-c2ccc(N(c3ccccc3)c3cc(N(c4ccccc4)c4ccc(-c5cc(-c6ccccc6)c6ccccc6c5)cc4)c4c(c3)oc3cc5ccccc5cc34)cc2)cc1]

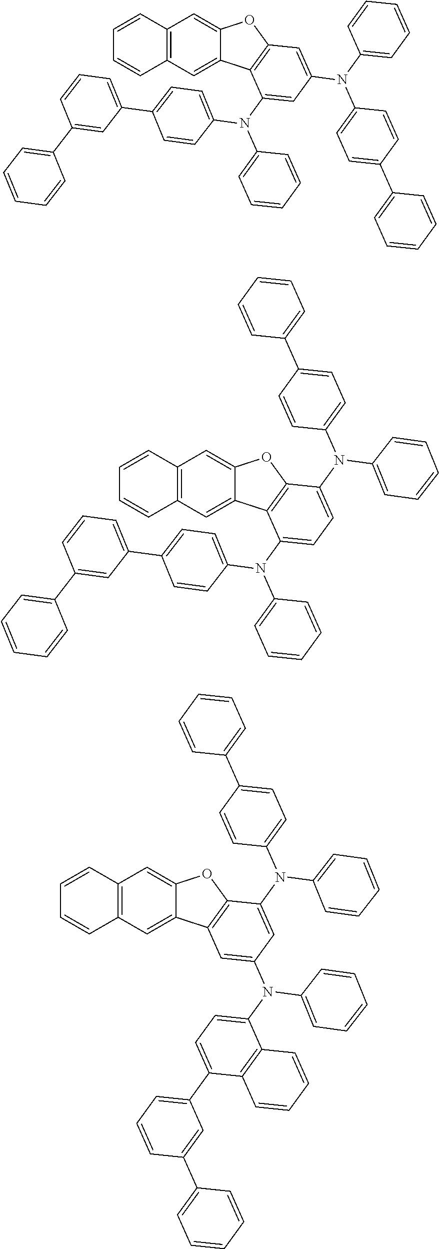 [SMILES: c1ccc(-c2ccc(N(c3ccccc3)c3cc(N(c4ccccc4)c4ccc(-c5cccc(-c6ccccc6)c5)cc4)c4c(c3)oc3cc5ccccc5cc34)cc2)cc1 ; c1ccc(-c2ccc(N(c3ccccc3)c3ccc(N(c4ccccc4)c4ccc(-c5cccc(-c6ccccc6)c5)cc4)c4c3oc3cc5ccccc5cc34)cc2)cc1 ; c1ccc(-c2ccc(N(c3ccccc3)c3cc(N(c4ccccc4)c4ccc(-c5cccc(-c6ccccc6)c5)c5ccccc45)cc4c3oc3cc5ccccc5cc34)cc2)cc1]

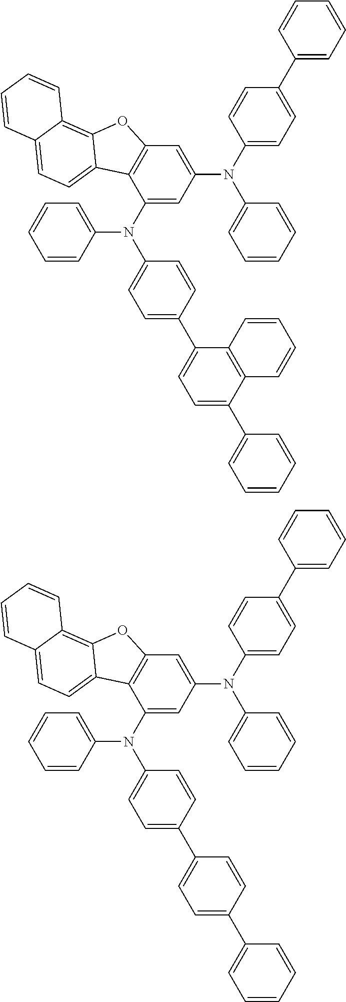

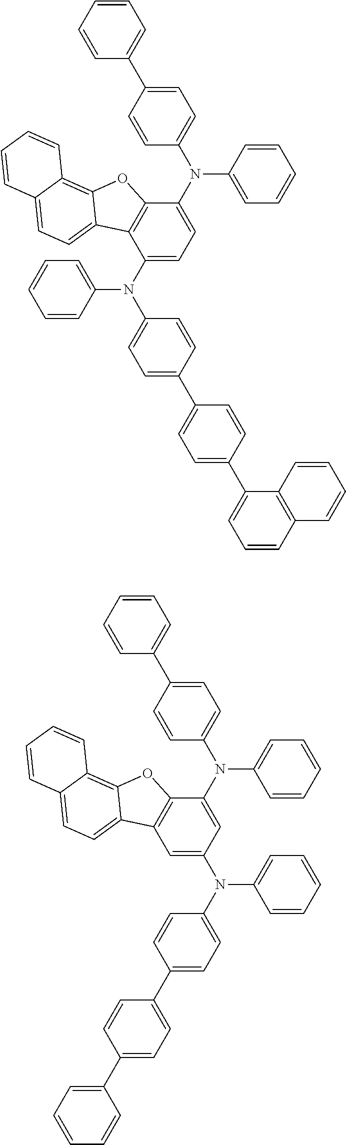 [SMILES: c1ccc(-c2ccc(N(c3ccccc3)c3ccc(N(c4ccccc4)c4ccc(-c5ccc(-c6cccc7ccccc67)cc5)cc4)c4c3oc3c5ccccc5ccc34)cc2)cc1 ; c1ccc(-c2ccc(-c3ccc(N(c4ccccc4)c4cc(N(c5ccccc5)c5ccc(-c6ccccc6)cc5)c5oc6c7ccccc7ccc6c5c4)cc3)cc2)cc1]

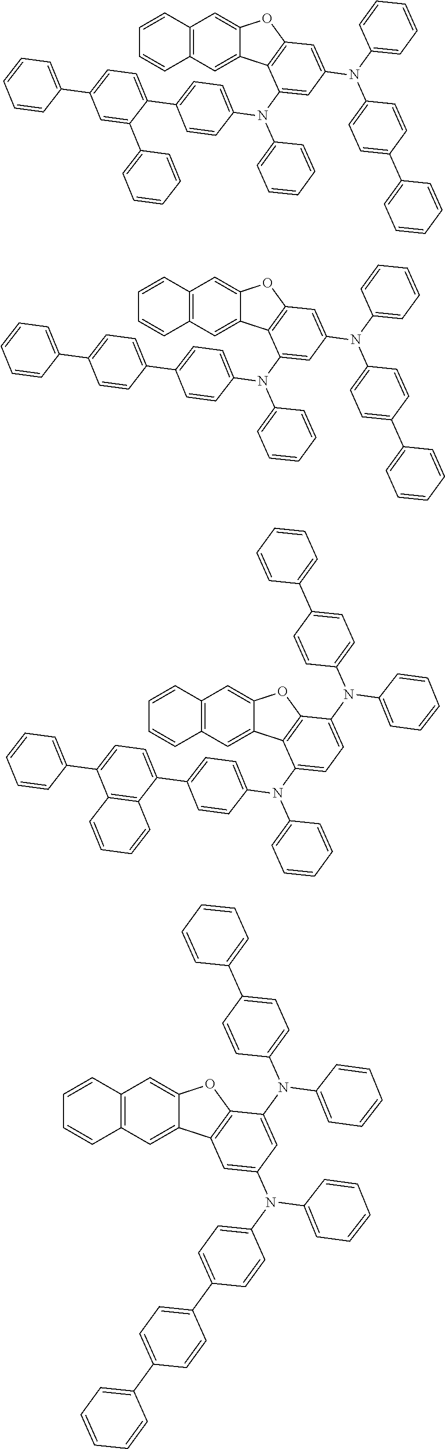

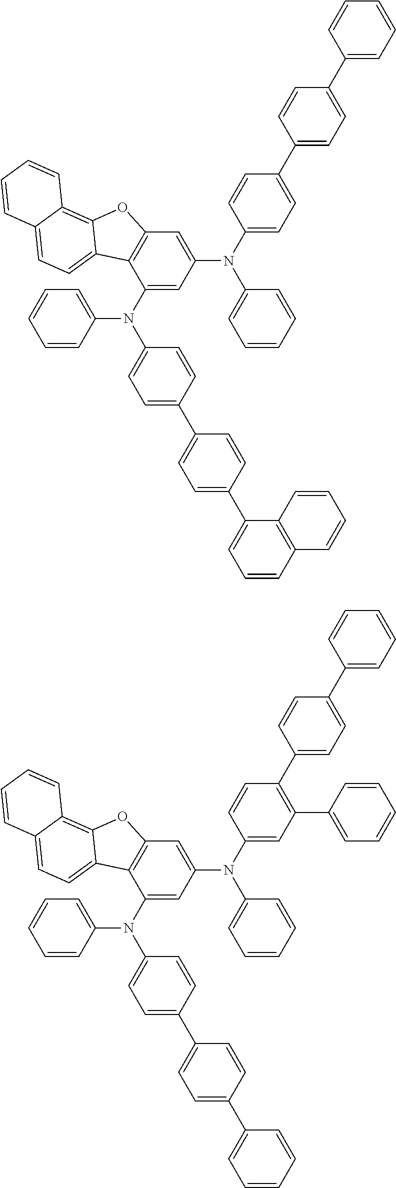 [SMILES: c1ccc(-c2ccc(-c3ccc(N(c4ccccc4)c4cc(N(c5ccccc5)c5ccc(-c6ccc(-c7cccc8ccccc78)cc6)cc5)c5c(c4)oc4c6ccccc6ccc45)cc3)cc2)cc1 ; c1ccc(-c2ccc(-c3ccc(N(c4ccccc4)c4cc(N(c5ccccc5)c5ccc(-c6ccc(-c7ccccc7)cc6)c(-c6ccccc6)c5)cc5oc6c7ccccc7ccc6c45)cc3)cc2)cc1]

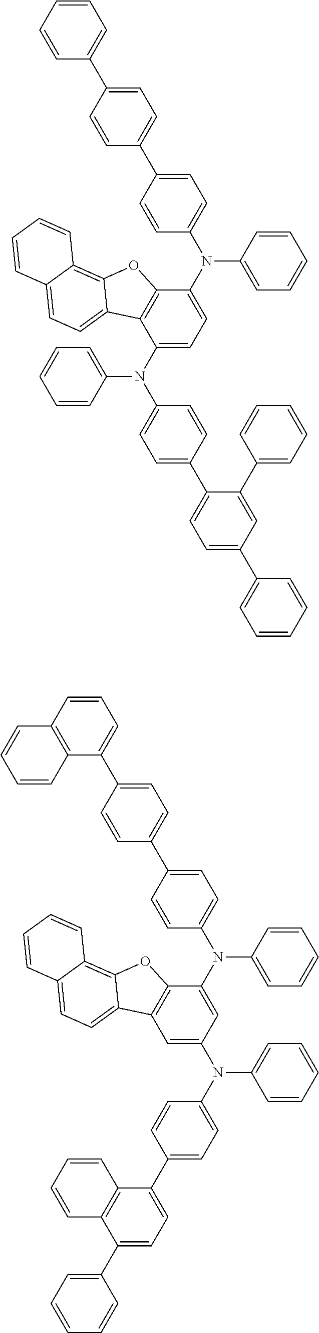 [SMILES: c1ccc(-c2ccc(-c3ccc(N(c4ccccc4)c4ccc(N(c5ccccc5)c5ccc(-c6ccc(-c7ccccc7)cc6-c6ccccc6)cc5)c5c4oc4c6ccccc6ccc45)cc3)cc2)cc1 ; c1ccc(-c2ccc(-c3ccc(N(c4ccccc4)c4cc(N(c5ccccc5)c5ccc(-c6ccc(-c7cccc8ccccc78)cc6)cc5)c5oc6c7ccccc7ccc6c5c4)cc3)c3ccccc23)cc1]

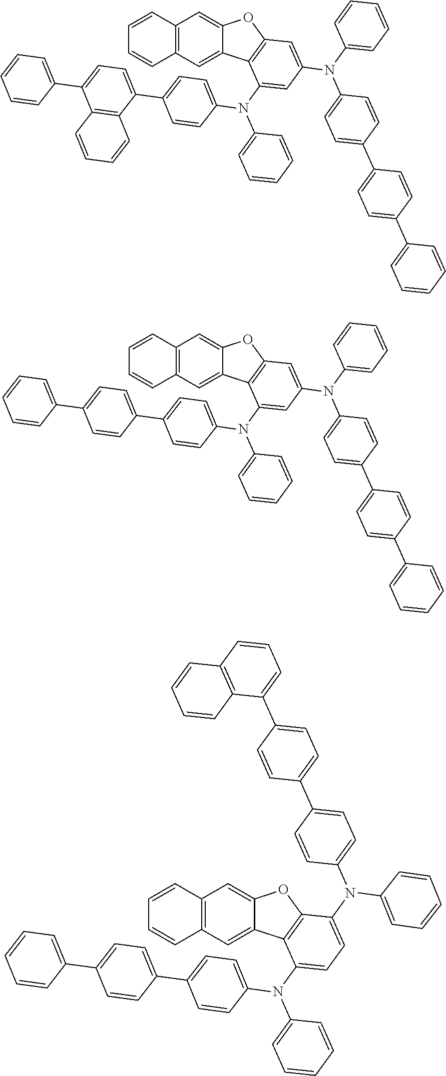 [SMILES: c1ccc(-c2ccc(-c3ccc(N(c4ccccc4)c4cc(N(c5ccccc5)c5ccc(-c6ccc(-c7ccccc7)c7ccccc67)cc5)c5c(c4)oc4cc6ccccc6cc45)cc3)cc2)cc1 ; c1ccc(-c2ccc(-c3ccc(N(c4ccccc4)c4cc(N(c5ccccc5)c5ccc(-c6ccc(-c7ccccc7)cc6)cc5)c5c(c4)oc4cc6ccccc6cc45)cc3)cc2)cc1 ; c1ccc(-c2ccc(-c3ccc(N(c4ccccc4)c4ccc(N(c5ccccc5)c5ccc(-c6ccc(-c7cccc8ccccc78)cc6)cc5)c5oc6cc7ccccc7cc6c45)cc3)cc2)cc1]

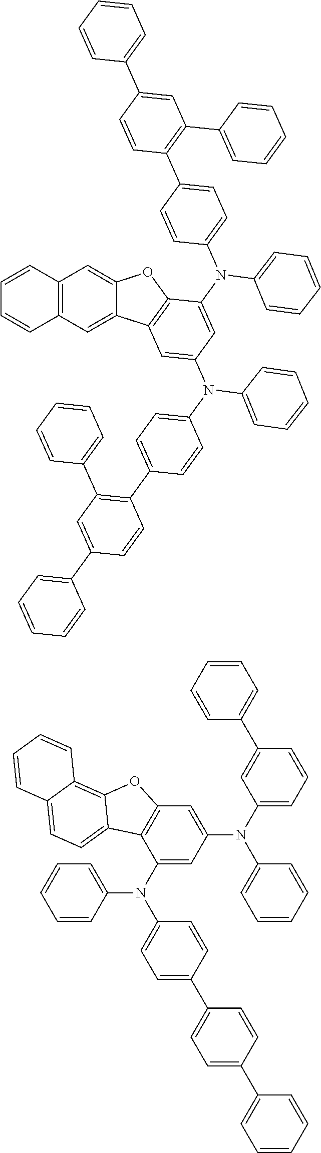 [SMILES: c1ccc(-c2ccc(-c3ccc(N(c4ccccc4)c4cc(N(c5ccccc5)c5ccc(-c6ccc(-c7ccccc7)cc6-c6ccccc6)cc5)c5oc6cc7ccccc7cc6c5c4)cc3)c(-c3ccccc3)c2)cc1 ; c1ccc(-c2ccc(-c3ccc(N(c4ccccc4)c4cc(N(c5ccccc5)c5cccc(-c6ccccc6)c5)cc5oc6c7ccccc7ccc6c45)cc3)cc2)cc1]

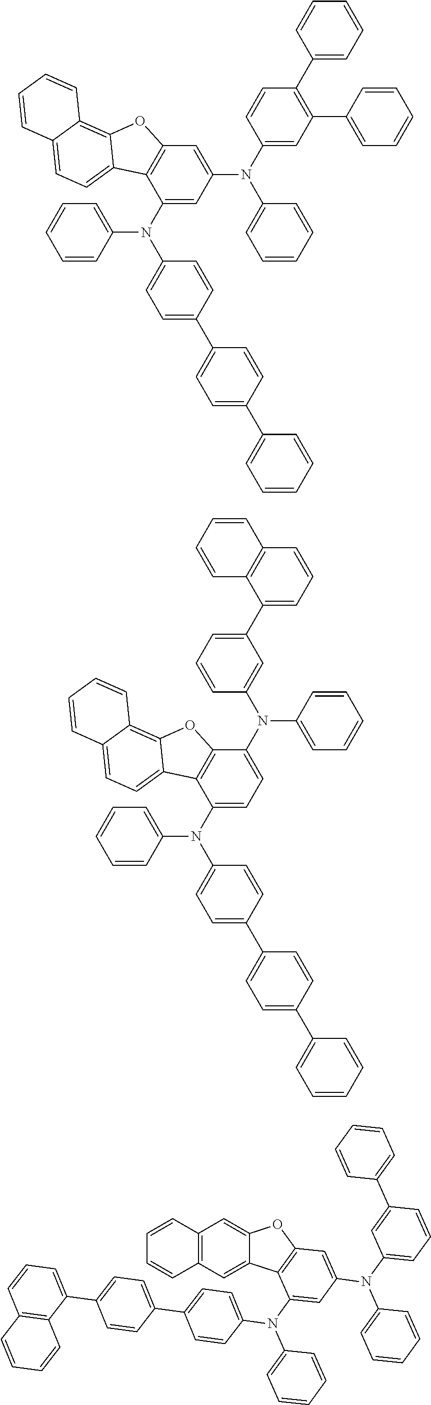

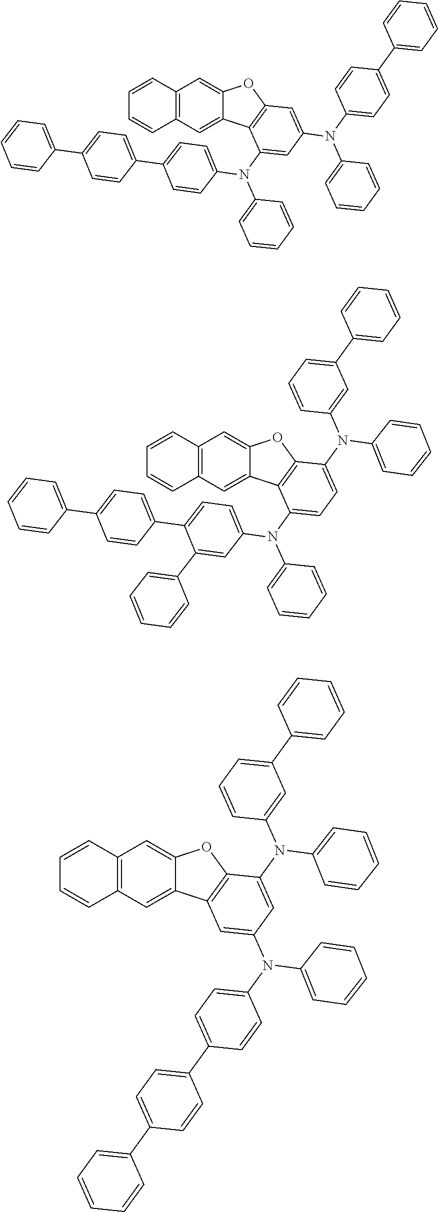 [SMILES: c1ccc(-c2ccc(-c3ccc(N(c4ccccc4)c4cc(N(c5ccccc5)c5ccc(-c6ccccc6)cc5)cc5oc6cc7ccccc7cc6c45)cc3)cc2)cc1 ; c1ccc(-c2ccc(-c3ccc(N(c4ccccc4)c4ccc(N(c5ccccc5)c5cccc(-c6ccccc6)c5)c5oc6cc7ccccc7cc6c45)cc3-c3ccccc3)cc2)cc1 ; c1ccc(-c2ccc(-c3ccc(N(c4ccccc4)c4cc(N(c5ccccc5)c5cccc(-c6ccccc6)c5)c5oc6cc7ccccc7cc6c5c4)cc3)cc2)cc1]

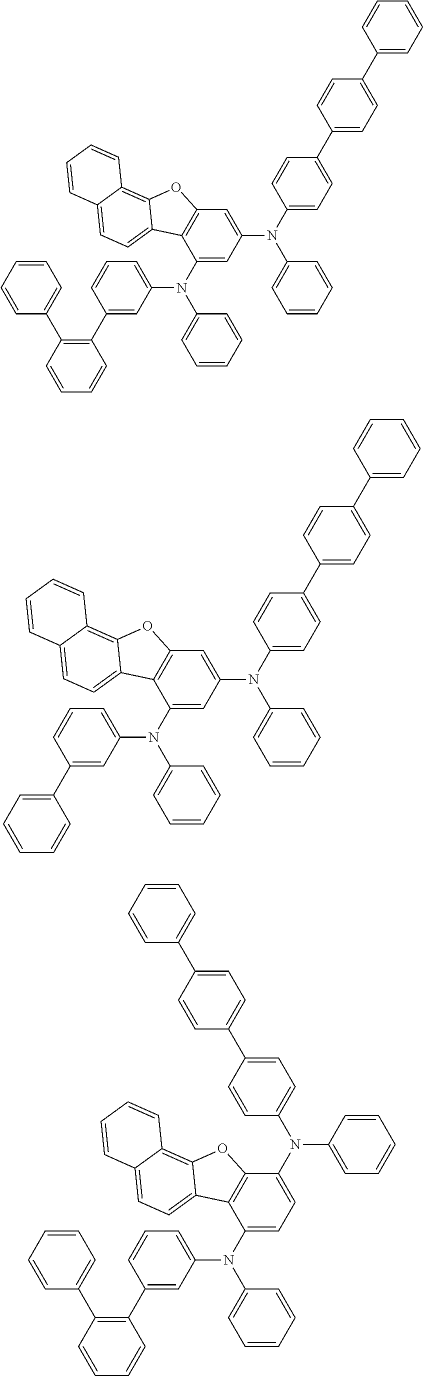 [SMILES: c1ccc(-c2ccc(-c3ccc(N(c4ccccc4)c4cc(N(c5ccccc5)c5cccc(-c6ccccc6-c6ccccc6)c5)c5c(c4)oc4c6ccccc6ccc45)cc3)cc2)cc1 ; c1ccc(-c2ccc(-c3ccc(N(c4ccccc4)c4cc(N(c5ccccc5)c5cccc(-c6ccccc6)c5)c5c(c4)oc4c6ccccc6ccc45)cc3)cc2)cc1 ; c1ccc(-c2ccc(-c3ccc(N(c4ccccc4)c4ccc(N(c5ccccc5)c5cccc(-c6ccccc6-c6ccccc6)c5)c5c4oc4c6ccccc6ccc45)cc3)cc2)cc1]

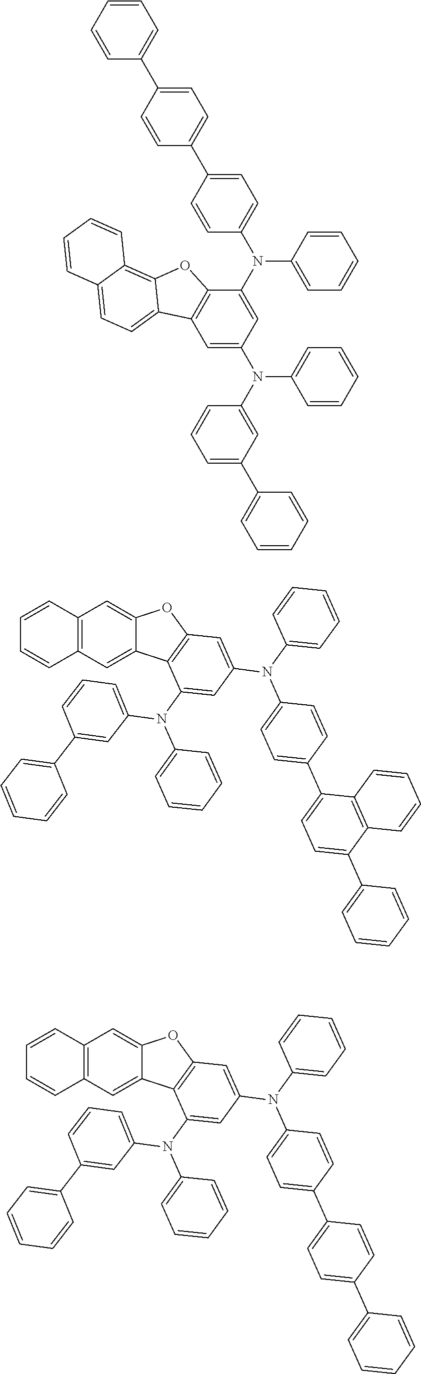

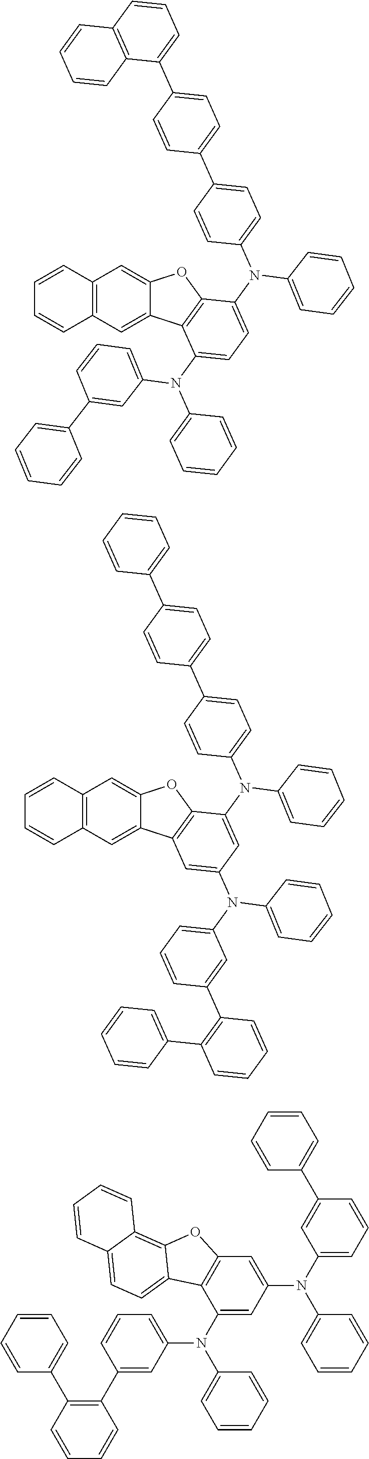

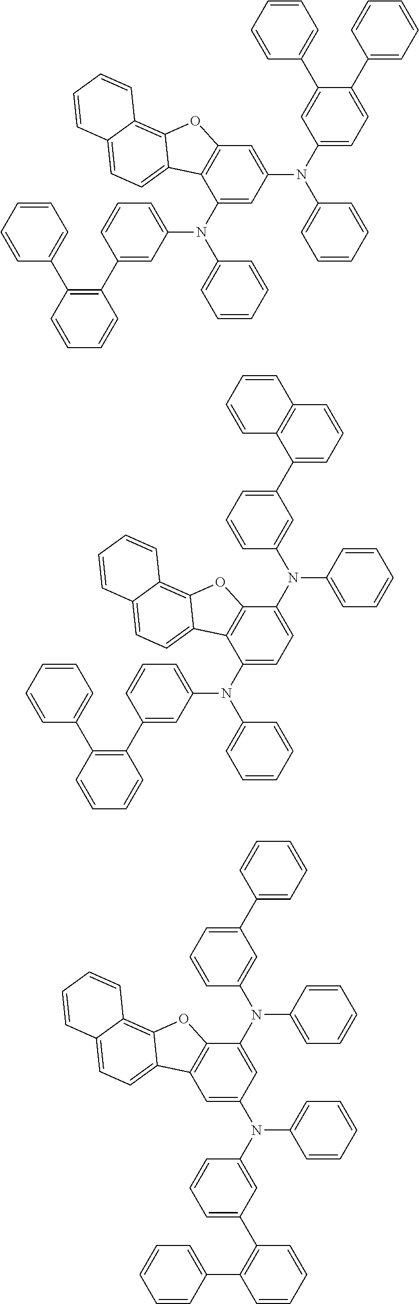 [SMILES: c1ccc(-c2ccccc2-c2cccc(N(c3ccccc3)c3cc(N(c4ccccc4)c4ccc(-c5ccccc5)c(-c5ccccc5)c4)cc4oc5c6ccccc6ccc5c34)c2)cc1 ; c1ccc(-c2ccccc2-c2cccc(N(c3ccccc3)c3ccc(N(c4ccccc4)c4cccc(-c5cccc6ccccc56)c4)c4oc5c6ccccc6ccc5c34)c2)cc1 ; c1ccc(-c2cccc(N(c3ccccc3)c3cc(N(c4ccccc4)c4cccc(-c5ccccc5-c5ccccc5)c4)cc4c3oc3c5ccccc5ccc43)c2)cc1]

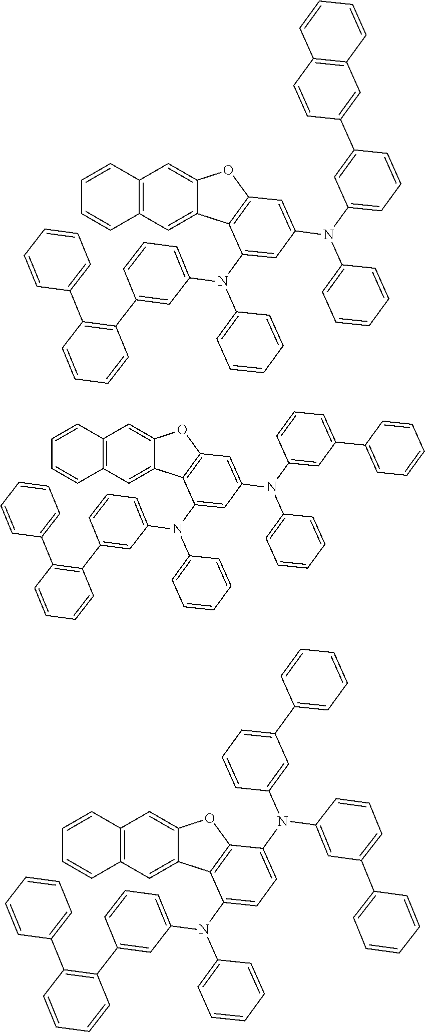

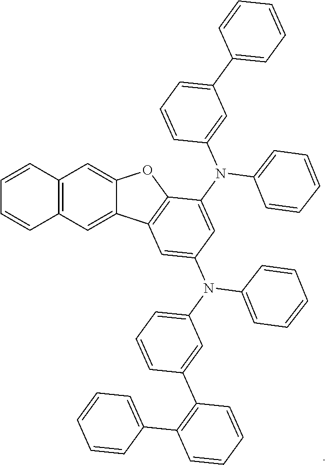 [SMILES: c1ccc(-c2cccc(N(c3ccccc3)c3cc(N(c4ccccc4)c4cccc(-c5ccccc5-c5ccccc5)c4)cc4c3oc3cc5ccccc5cc34)c2)cc1]

In addition, according to the present disclosure, there is provided a method for preparing the compound represented by Chemical Formula 2.

In one example, the compound represented by Chemical Formula 2 can be prepared by a preparation method as shown in the following Reaction Scheme 2.

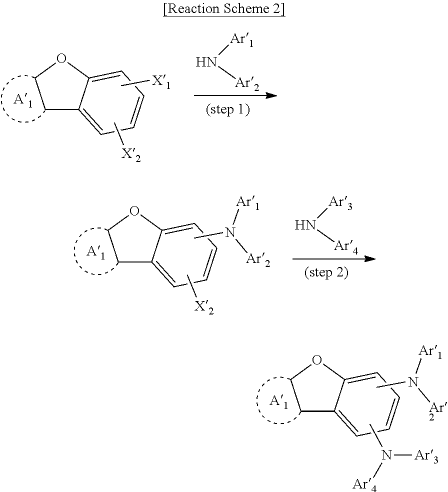

wherein, the definition of the remaining substituents except for X′₁ and X′₂ are the same as defined in the Chemical Formula 2, and X′₁ and X′₂ are each independently halogen, preferably chloro or bromo.

The Reaction Scheme 2 is an amine substitution reaction, which is preferably carried out in the presence of a palladium catalyst and a base, and a reactive group for the amine substitution reaction can be changed as known in the art.

The preparation method can be further embodied in Preparation Examples described hereinafter.

In the light emitting layer, the weight ratio between the compound represented by Chemical Formula 1 and the compound represented by Chemical Formula 2 is 1:99 to 99:1, 5:95 to 95:5, or 10:90 to 90:10.

The dopant material is not particularly limited as long as it is a material used for the organic light emitting device. As an example, an aromatic amine derivative, a styrylamine compound, a boron complex, a fluoranthene compound, a metal complex, and the like can be mentioned. Specific examples of the aromatic amine derivatives include substituted or unsubstituted fused aromatic ring derivatives having an arylamino group, examples thereof include pyrene, anthracene, chrysene, and periflanthene having the arylamino group, and the like. The styrylamine compound is a compound of an arylamine, which is unsubstituted or substituted with one or two or more substituent groups selected from the group consisting of an aryl group, a silyl group, an alkyl group, a cycloalkyl group, and an arylamino group, is substituted with at least one arylvinyl group. Specific examples thereof include styrylamine, styryldiamine, styryltriamine, styryltetramine, and the like, but are not limited thereto. Further, examples of the metal complex include an iridium complex, a platinum complex, and the like, but are not limited thereto.

Specific examples of the dopant material may include the following compounds, but are not limited thereto:

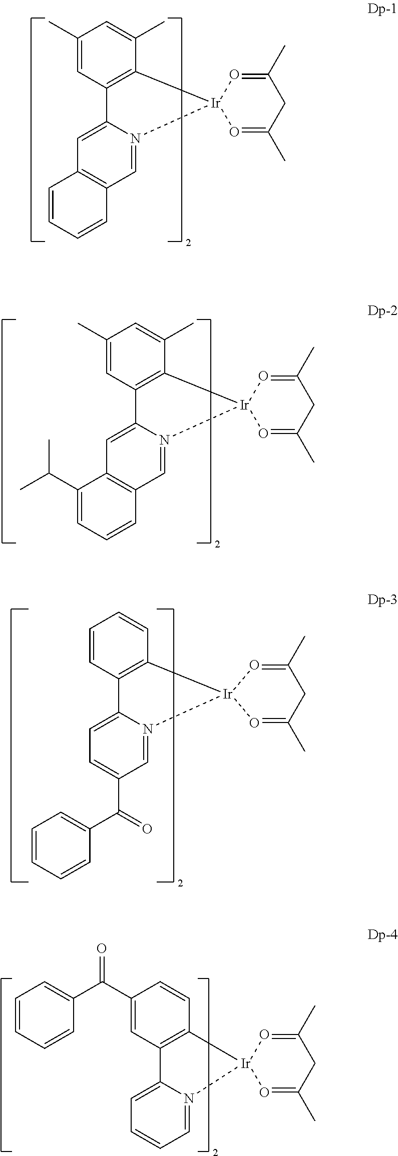

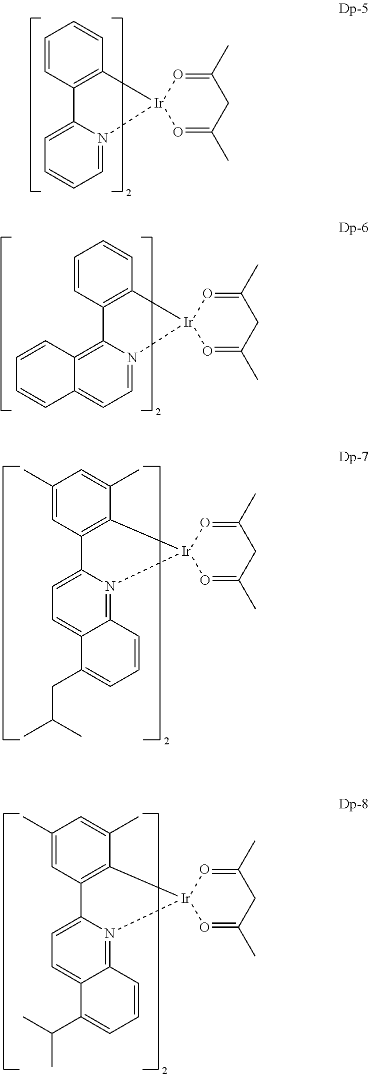

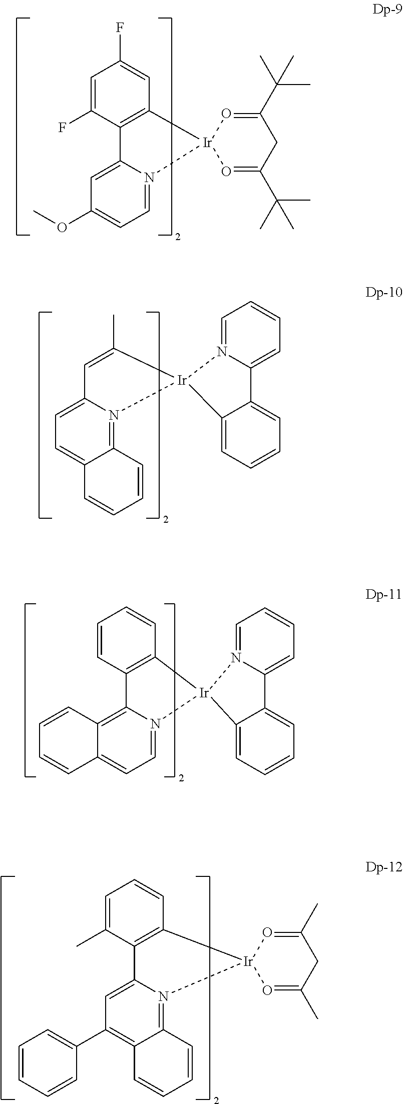

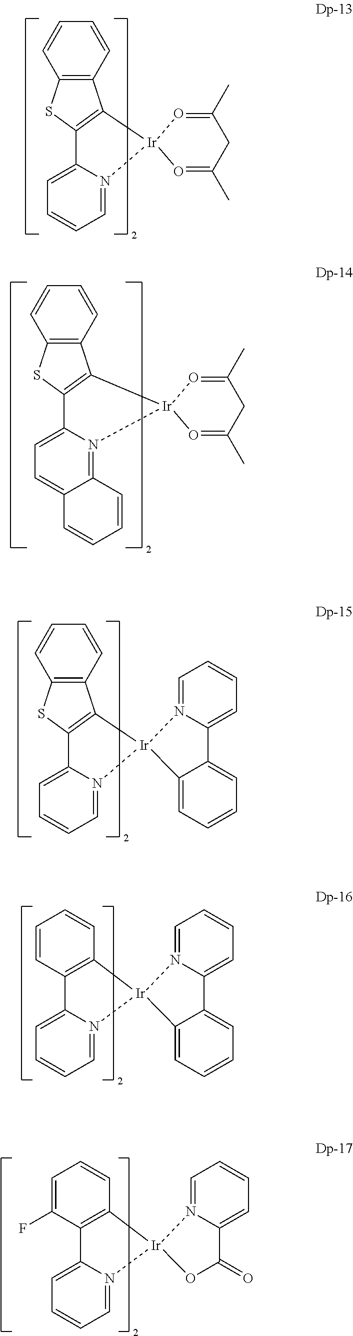

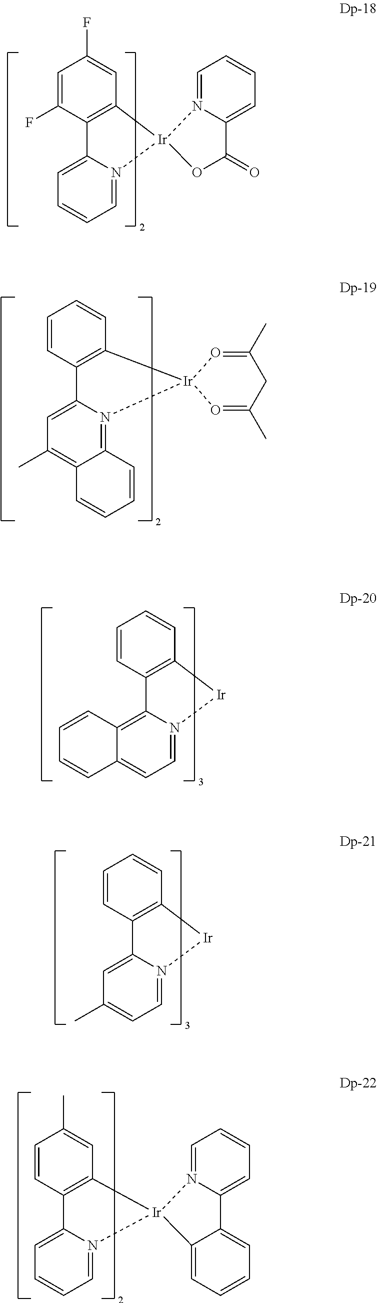

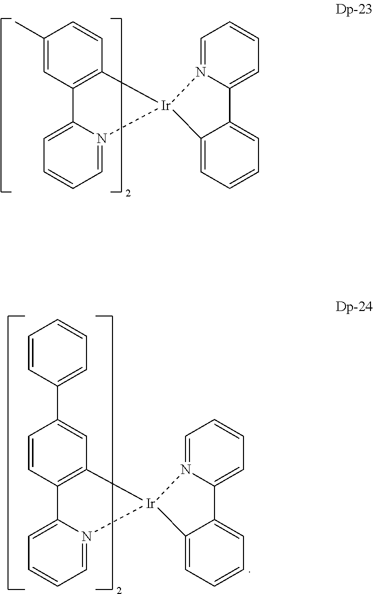

Hole Transport Layer

The organic light emitting device according to the present disclosure may include a hole transport layer between the light emitting layer and the anode.

The hole transport layer is a layer that receives holes from a hole injection layer and transports the holes to the light emitting layer. The hole transport material is suitably a material having large mobility to the holes, which may receive holes from the anode or the hole injection layer and transfer the holes to the light emitting layer.

Specific examples of the hole transport material include an arylamine-based organic material, a conductive polymer, a block copolymer in which a conjugate portion and a non-conjugate portion are present together, and the like, but are not limited thereto.

Hole Injection Layer

The organic light emitting device according to the present disclosure may further include a hole injection layer between the anode and the hole transport layer, if necessary.

The hole injection layer is a layer injecting holes from an electrode, and the hole injection material is preferably a compound which has a capability of transporting the holes, has a hole injection effect in the anode and an excellent hole injection effect to the light emitting layer or the light emitting material, prevents movement of an exciton generated in the light emitting layer to the electron injection layer or the electron injection material, and is excellent in the ability to form a thin film. Further, it is preferable that a HOMO (highest occupied molecular orbital) of the hole injection material is between the work function of the anode material and a HOMO of a peripheral organic material layer.

Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

Electron Blocking Layer

The organic light emitting device according to the present disclosure includes an electron blocking layer between the hole transport layer and the light emitting layer, if necessary.

The electron blocking layer prevents the electrons injected from the cathode from being transferred to the hole transport layer without being recombined in the light emitting layer, which may also be referred to as an electron inhibition layer. The electron blocking layer is preferably a material having the smaller electron affinity than the electron transport layer.

Electron Transport Layer

The organic light emitting device according to the present disclosure may include an electron transport layer between the light emitting layer and the cathode.

The electron transport layer is a layer that receives the electrons from the electron injection layer formed on the cathode or the cathode and transports the electrons to the light emitting layer, and that suppress the transfer of holes from the light emitting layer, and an electron transport material is suitably a material which may receive electrons well from a cathode and transfer the electrons to a light emitting layer, and has a large mobility for electrons.

Specific examples of the electron transport material include: an Al complex of 8-hydroxyquinoline; a complex including Alq3; an organic radical compound; a hydroxyflavone-metal complex, and the like, but are not limited thereto. The electron transport layer may be used with any desired cathode material, as used according to a conventional technique. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

Electron Injection Layer

The organic light emitting device according to the present disclosure may further include an electron injection layer between the electron transport layer and the cathode, if necessary.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film.

Specific examples of the materials that can be used as the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

According to one embodiment of the present disclosure, the electron transport material and the electron injection material may be simultaneously deposited to produce a single layer of the electron injection and transport layer.

Hole Blocking Layer

The organic light emitting device according to the present disclosure may include a hole blocking layer between the electron transport layer and the light emitting layer, if necessary.

The hole blocking layer prevents the holes injected from the anode from being transferred to the electron transport layer without being recombined in the light emitting layer, and the hole blocking layer is preferably a material having high ionization energy.

Organic Light Emitting Device

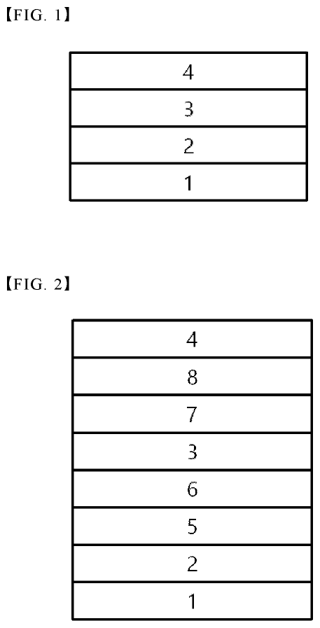

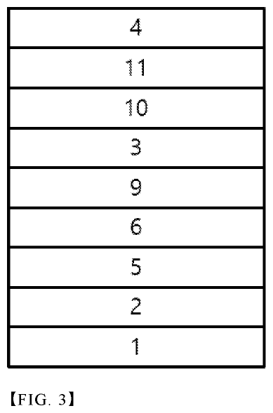

The structure of the organic light emitting device according to the present disclosure is illustrated in FIG. 1. FIG. 1 shows an example of an organic light emitting device comprising a substrate 1, an anode 2, a light emitting layer 3, and a cathode 4. Also, FIG. 2 shows an example of an organic light emitting device comprising a substrate 1, an anode 2, a hole injection layer 5, a hole transport layer 6, a light emitting layer 3, an electron transport layer 7, an electron injection layer 8 and a cathode 4. In addition, FIG. 3 shows an example of an organic light emitting device comprising a substrate 1, an anode 2, a hole injection layer 5, a hole transport layer 6, an electron blocking layer 9, a light emitting layer 3, a hole blocking layer 10, an electron injection and transport layer 11, and a cathode 4.

The organic light emitting device according to the present disclosure can be manufactured by sequentially stacking the above-described structures. In this case, the organic light emitting device may be manufactured by depositing a metal, metal oxides having conductivity, or an alloy thereof on the substrate by using a PVD (physical vapor deposition) method such as a sputtering method or an e-beam evaporation method to form the anode, forming the respective layers described above thereon, and then depositing a material that can be used as the cathode thereon.

In addition to such a method, the organic light emitting device may be manufactured by sequentially depositing from the cathode material to the anode material on a substrate in the reverse order of the above-mentioned configuration. (WO 2003/012890) Further, the light emitting layer may be formed by subjecting hosts and dopants to a vacuum deposition method and a solution coating method. Herein, the solution coating method means a spin coating, a dip coating, a doctor blading, an inkjet printing, a screen printing, a spray method, a roll coating, or the like, but is not limited thereto.

Meanwhile, the organic light emitting device according to the present disclosure may be a bottom emission device, a top emission device, or a double-sided light emitting device depending on the materials used.

The preparation of the organic light emitting device according to the present disclosure will be described in detail in the following examples. However, these examples are presented for illustrative purposes only, and are not intended to limit the scope of the present disclosure.

### PREPARATION EXAMPLES

### Preparation Example 1: Preparation of Compound of Chemical Formula 1

### Preparation Example 1-1

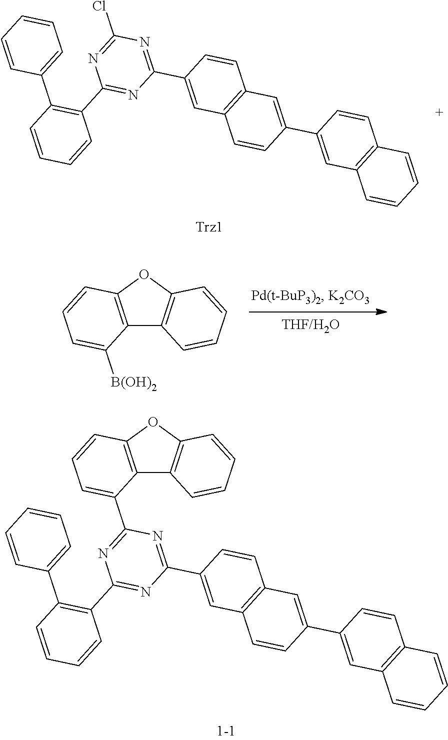 [SMILES: Clc1nc(-c2ccc3cc(-c4ccc5ccccc5c4)ccc3c2)nc(-c2ccccc2-c2ccccc2)n1 ; OB(O)c1cccc2oc3ccccc3c12 ; c1ccc(-c2ccccc2-c2nc(-c3ccc4cc(-c5ccc6ccccc6c5)ccc4c3)nc(-c3cccc4oc5ccccc5c34)n2)cc1]

Trz1 (15 g, 28.8 mmol) and dibenzo[b,d]furan-1-ylboronic acid (6.4 g, 30.3 mmol) were added to 300 ml of THF, and the mixture was stirred and refluxed. Then, potassium carbonate (12 g, 86.5 mmol) was dissolved in 36 ml of water and added thereto, and the mixture was sufficiently stirred, and then bis(tri-tert-butylphosphine)palladium(0) (0.1 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.2 g of Compound 1-1 (Yield: 65%, MS: [M+H]⁺=652)

### Preparation Example 1-2

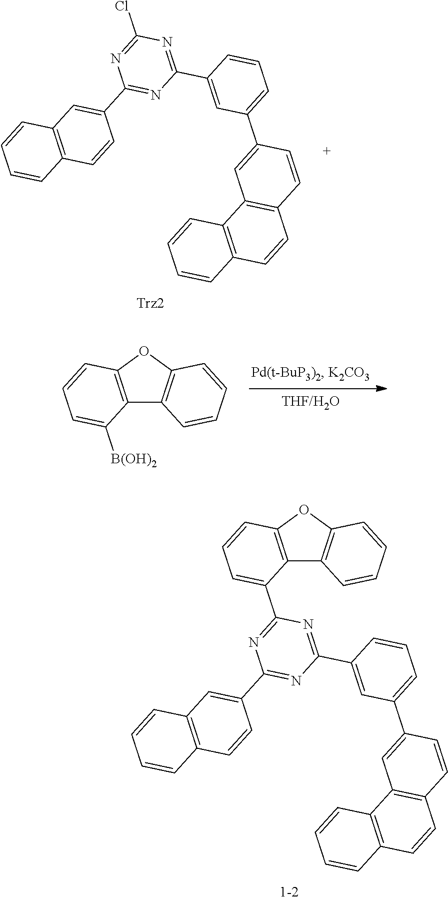 [SMILES: Clc1nc(-c2cccc(-c3ccc4ccc5ccccc5c4c3)c2)nc(-c2ccc3ccccc3c2)n1 ; OB(O)c1cccc2oc3ccccc3c12 ; c1cc(-c2ccc3ccc4ccccc4c3c2)cc(-c2nc(-c3ccc4ccccc4c3)nc(-c3cccc4oc5ccccc5c34)n2)c1]

Trz2 (15 g, 30.4 mmol) and dibenzo[b,d]furan-1-ylboronic acid (6.8 g, 31.9 mmol) were added to 300 ml of THF, and the mixture was stirred and refluxed. Then, potassium carbonate (12.6 g, 91.1 mmol) was dissolved in 38 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 14 g of Compound 1-2 (Yield: 74%, MS: [M+H]+=626)

### Preparation Example 1-3

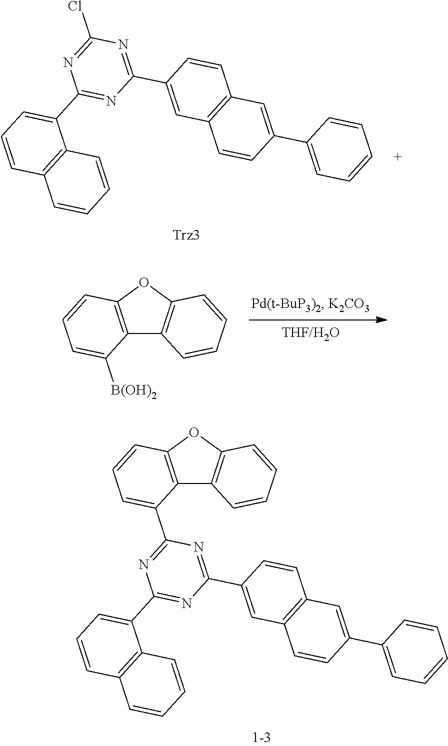 [SMILES: Clc1nc(-c2ccc3cc(-c4ccccc4)ccc3c2)nc(-c2cccc3ccccc23)n1 ; OB(O)c1cccc2oc3ccccc3c12 ; c1ccc(-c2ccc3cc(-c4nc(-c5cccc6ccccc56)nc(-c5cccc6oc7ccccc7c56)n4)ccc3c2)cc1]

Trz3 (15 g, 33.8 mmol) and dibenzo[b,d]furan-1-ylboronic acid (7.5 g, 35.5 mmol) were added to 300 ml of THF, and the mixture was stirred and refluxed. Then, potassium carbonate (14 g, 101.4 mmol) was dissolved in 42 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.4 g of Compound 1-3 (Yield: 69%, MS: [M+H]+=576)

### Preparation Example 1-4

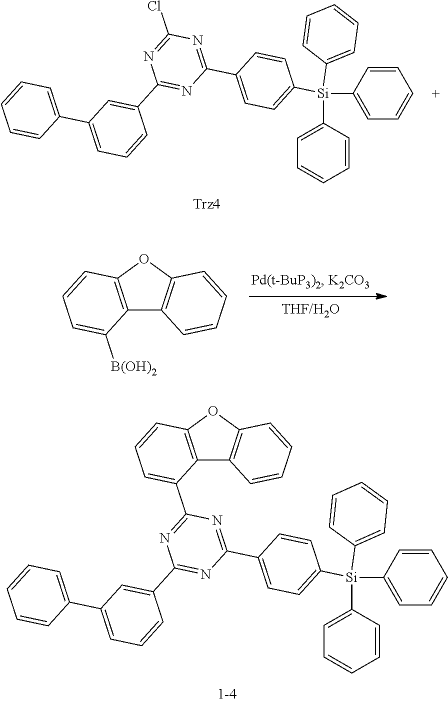 [SMILES: Clc1nc(-c2ccc([Si](c3ccccc3)(c3ccccc3)c3ccccc3)cc2)nc(-c2cccc(-c3ccccc3)c2)n1 ; OB(O)c1cccc2oc3ccccc3c12 ; c1ccc(-c2cccc(-c3nc(-c4ccc([Si](c5ccccc5)(c5ccccc5)c5ccccc5)cc4)nc(-c4cccc5oc6ccccc6c45)n3)c2)cc1]

Trz4 (15 g, 24.9 mmol) and dibenzo[b,d]furan-1-ylboronic acid (5.5 g, 26.2 mmol) were added to 300 ml of THF, and the mixture was stirred and refluxed. Then, potassium carbonate (10.3 g, 74.7 mmol) was dissolved in 31 ml of water and added thereto, and the mixture was sufficiently stirred, and then bis(tri-tert-butylphosphine)palladium(0) (0.1 g, 0.2 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.6 g of Compound 1-4 (Yield: 69%, MS: [M+H]+=734)

### Preparation Example 1-5

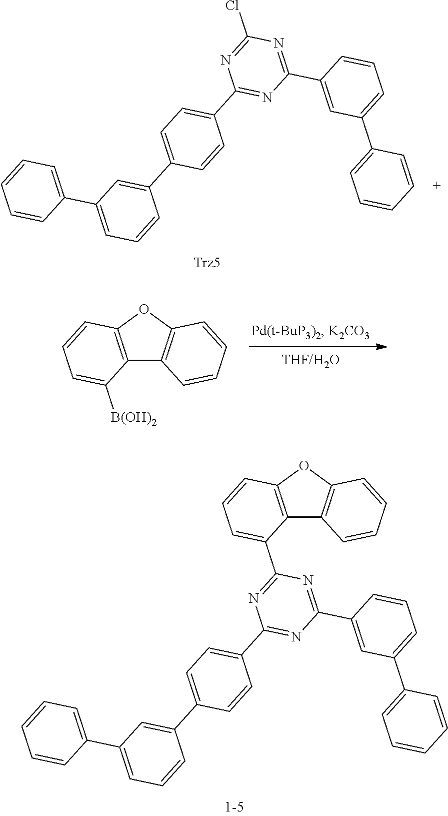 [SMILES: Clc1nc(-c2ccc(-c3cccc(-c4ccccc4)c3)cc2)nc(-c2cccc(-c3ccccc3)c2)n1 ; OB(O)c1cccc2oc3ccccc3c12 ; c1ccc(-c2cccc(-c3ccc(-c4nc(-c5cccc(-c6ccccc6)c5)nc(-c5cccc6oc7ccccc7c56)n4)cc3)c2)cc1]

Trz5 (15 g, 30.2 mmol) and dibenzo[b,d]furan-1-ylboronic acid (6.7 g, 31.8 mmol) were added to 300 ml of THF, and the mixture was stirred and refluxed. Then, potassium carbonate (12.5 g, 90.7 mmol) was dissolved in 38 ml of water and added thereto, and the mixture was sufficiently stirred, and then bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.5 g of Compound 1-5 (Yield: 66%, MS: [M+H]+=629)

### Preparation Example 1-6

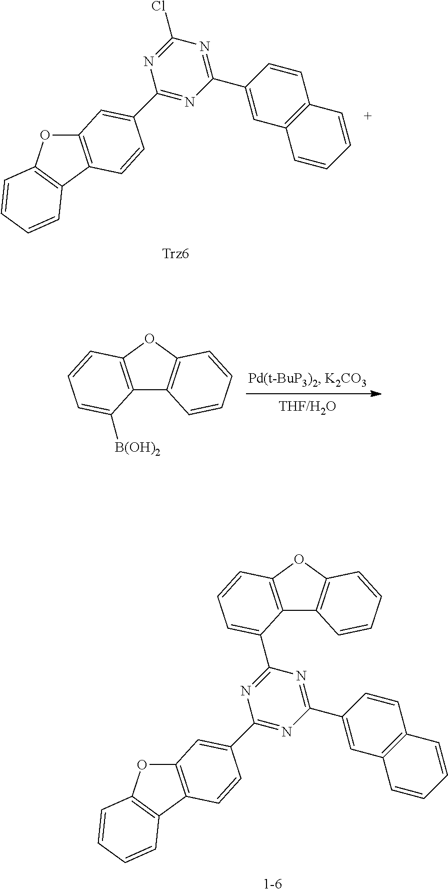 [SMILES: Clc1nc(-c2ccc3ccccc3c2)nc(-c2ccc3c(c2)oc2ccccc23)n1 ; OB(O)c1cccc2oc3ccccc3c12 ; c1ccc2cc(-c3nc(-c4ccc5c(c4)oc4ccccc45)nc(-c4cccc5oc6ccccc6c45)n3)ccc2c1]

Trz6 (15 g, 36.8 mmol) and dibenzo[b,d]furan-1-ylboronic acid (8.2 g, 38.6 mmol) were added to 300 ml of THF, and the mixture was stirred and refluxed. Then, potassium carbonate (15.2 g, 110.3 mmol) was dissolved in 46 ml of water and added thereto, and the mixture was sufficiently stirred, and then bis(tri-tert-butylphosphine)palladium(0) (0.2 g, 0.4 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 14.9 g of Compound 1-6 (Yield: 75%, MS: [M+H]+=540)

### Preparation Example 1-7

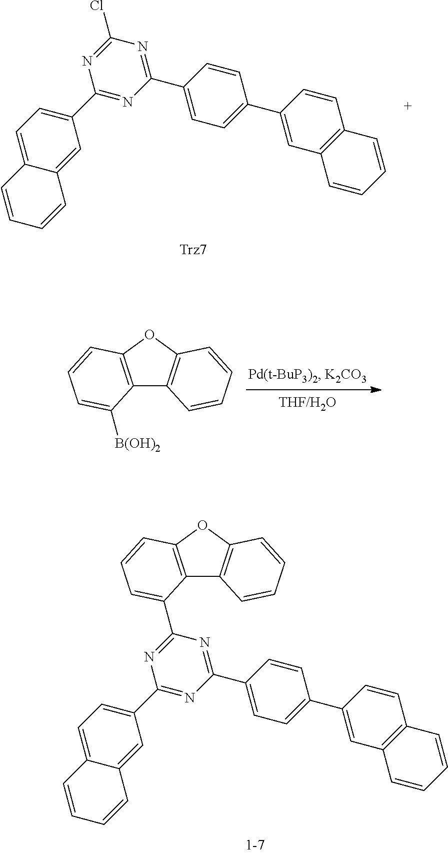 [SMILES: Clc1nc(-c2ccc(-c3ccc4ccccc4c3)cc2)nc(-c2ccc3ccccc3c2)n1 ; OB(O)c1cccc2oc3ccccc3c12 ; c1ccc2cc(-c3ccc(-c4nc(-c5ccc6ccccc6c5)nc(-c5cccc6oc7ccccc7c56)n4)cc3)ccc2c1]

Trz7 (15 g, 33.8 mmol) and dibenzo[b,d]furan-1-ylboronic acid (7.5 g, 35.5 mmol) were added to 300 ml of THF, and the mixture was stirred and refluxed. Then, potassium carbonate (14 g, 101.4 mmol) was dissolved in 42 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.6 g of Compound 1-7 (Yield: 70%, MS: [M+H]+=576)

### Preparation Example 1-8

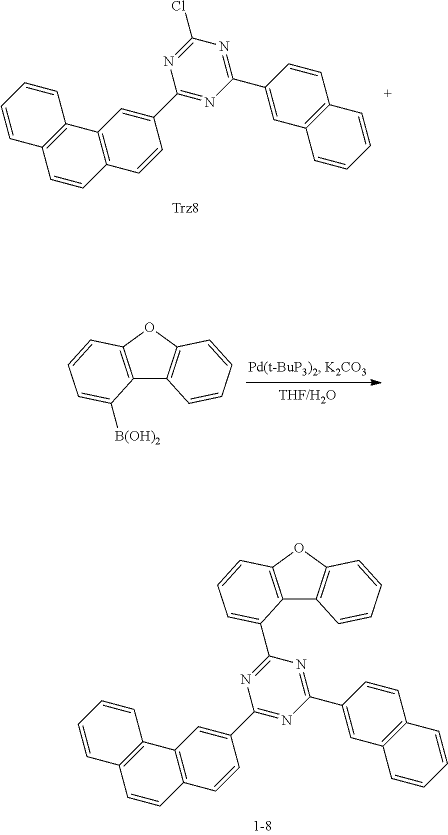 [SMILES: Clc1nc(-c2ccc3ccccc3c2)nc(-c2ccc3ccc4ccccc4c3c2)n1 ; OB(O)c1cccc2oc3ccccc3c12 ; c1ccc2cc(-c3nc(-c4ccc5ccc6ccccc6c5c4)nc(-c4cccc5oc6ccccc6c45)n3)ccc2c1]

Trz8 (15 g, 35.9 mmol) and dibenzo[b,d]furan-1-ylboronic acid (8 g, 37.7 mmol) were added to 300 ml of THF, and the mixture was stirred and refluxed. Then, potassium carbonate (14.9 g, 107.7 mmol) was dissolved in 45 ml of water and added thereto, and the mixture was sufficiently stirred, and then bis(tri-tert-butylphosphine)palladium(0) (0.2 g, 0.4 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.8 g of Compound 1-8 (Yield: 70%, MS: [M+H]+=550)

### Preparation Example 1-9

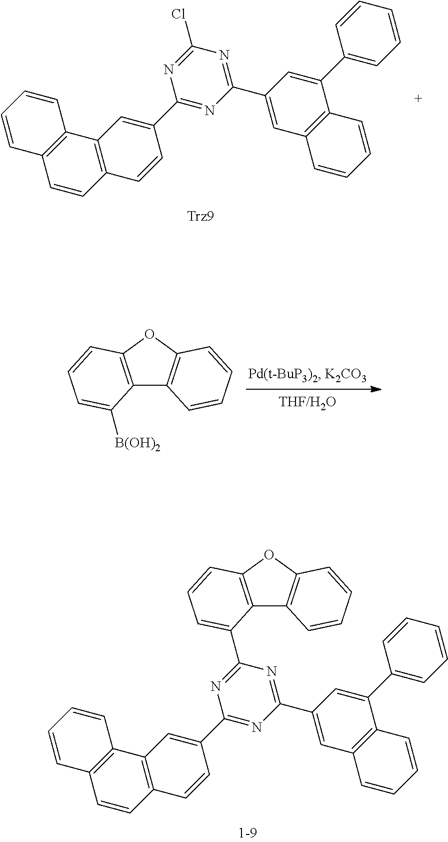 [SMILES: Clc1nc(-c2cc(-c3ccccc3)c3ccccc3c2)nc(-c2ccc3ccc4ccccc4c3c2)n1 ; OB(O)c1cccc2oc3ccccc3c12 ; c1ccc(-c2cc(-c3nc(-c4ccc5ccc6ccccc6c5c4)nc(-c4cccc5oc6ccccc6c45)n3)cc3ccccc23)cc1]

Trz9 (15 g, 30.4 mmol) and dibenzo[b,d]furan-1-ylboronic acid (6.8 g, 31.9 mmol) were added to 300 ml of THF, and the mixture was stirred and refluxed. Then, potassium carbonate (12.6 g, 91.1 mmol) was dissolved in 38 ml of water and added thereto, and the mixture was sufficiently stirred, and then bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.7 g of Compound 1-9 (Yield: 72%, MS: [M+H]+=626)

### Preparation Example 1-10

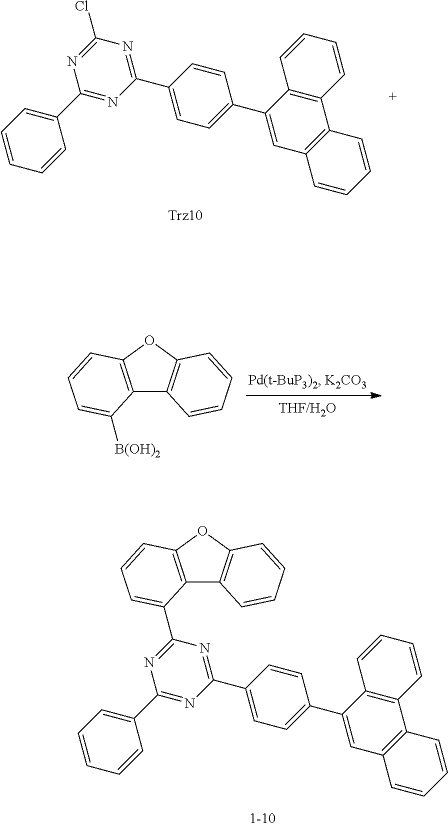 [SMILES: Clc1nc(-c2ccccc2)nc(-c2ccc(-c3cc4ccccc4c4ccccc34)cc2)n1 ; OB(O)c1cccc2oc3ccccc3c12 ; c1ccc(-c2nc(-c3ccc(-c4cc5ccccc5c5ccccc45)cc3)nc(-c3cccc4oc5ccccc5c34)n2)cc1]

Trz10 (15 g, 33.8 mmol) and dibenzo[b,d]furan-1-ylboronic acid (7.5 g, 35.5 mmol) were added to 300 ml of THF, and the mixture was stirred and refluxed. Then, potassium carbonate (14 g, 101.4 mmol) was dissolved in 42 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 14.2 g of Compound 1-10 (Yield: 73%, MS: [M+H]+=576)

### Preparation Example 1-11

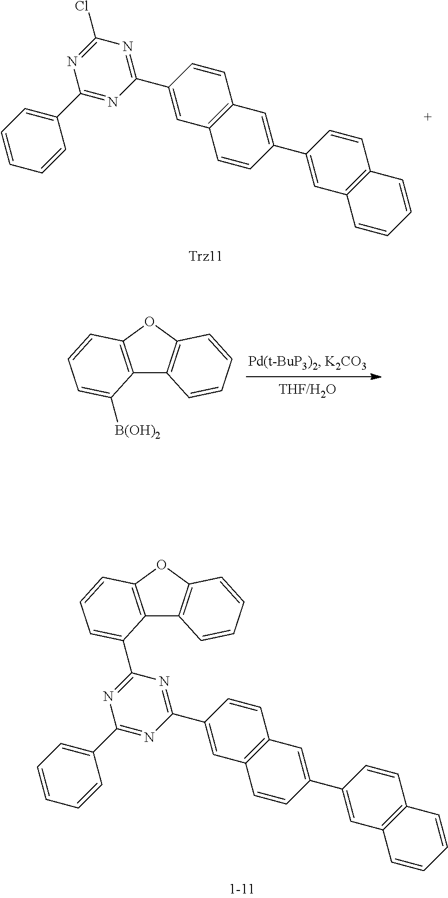 [SMILES: Clc1nc(-c2ccccc2)nc(-c2ccc3cc(-c4ccc5ccccc5c4)ccc3c2)n1 ; OB(O)c1cccc2oc3ccccc3c12 ; c1ccc(-c2nc(-c3ccc4cc(-c5ccc6ccccc6c5)ccc4c3)nc(-c3cccc4oc5ccccc5c34)n2)cc1]

Trz11 (15 g, 33.8 mmol) and dibenzo[b,d]furan-1-ylboronic acid (7.5 g, 35.5 mmol) were added to 300 ml of THF, and the mixture was stirred and refluxed. Then, potassium carbonate (14 g, 101.4 mmol) was dissolved in 42 ml of water and added thereto, and the mixture was sufficiently stirred, and then bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.4 g of Compound 1-11 (Yield: 69%, MS: [M+H]+=576)

### Preparation Example 1-12

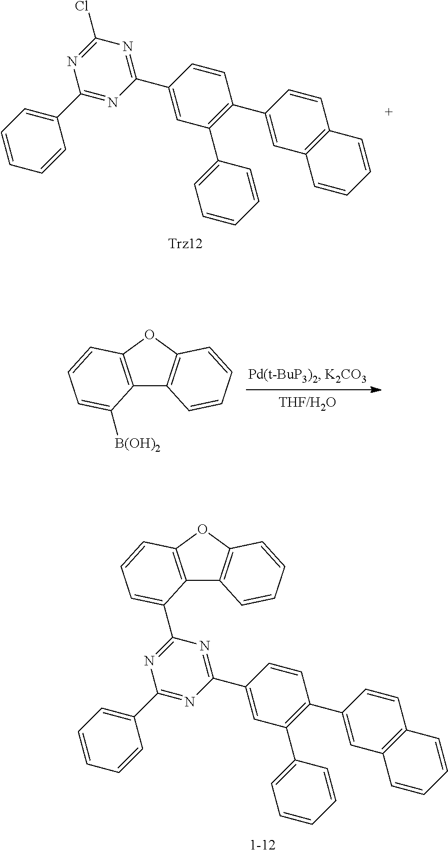 [SMILES: Clc1nc(-c2ccccc2)nc(-c2ccc(-c3ccc4ccccc4c3)c(-c3ccccc3)c2)n1 ; OB(O)c1cccc2oc3ccccc3c12 ; c1ccc(-c2nc(-c3ccc(-c4ccc5ccccc5c4)c(-c4ccccc4)c3)nc(-c3cccc4oc5ccccc5c34)n2)cc1]

Trz12 (15 g, 31.9 mmol) and dibenzo[b,d]furan-1-ylboronic acid (7.1 g, 33.5 mmol) were added to 300 ml of THF, and the mixture was stirred and refluxed. Then, potassium carbonate (13.2 g, 95.8 mmol) was dissolved in 40 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 14.2 g of Compound 1-12 (Yield: 74%, MS: [M+H]+=602)

### Preparation Example 1-13

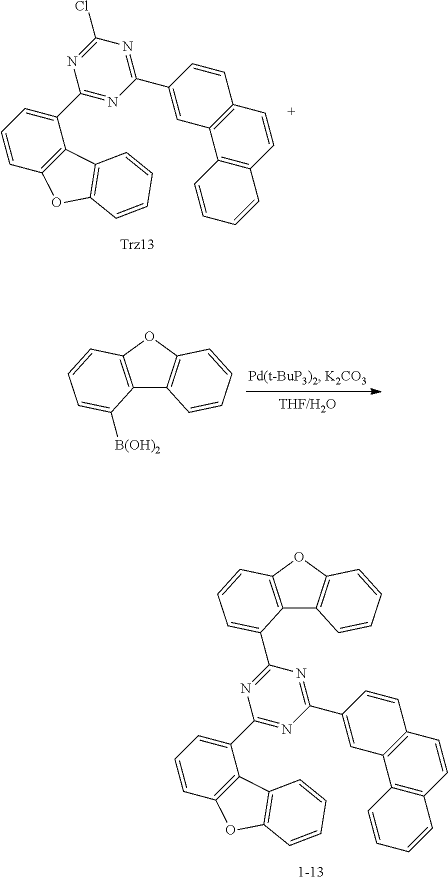 [SMILES: Clc1nc(-c2ccc3ccc4ccccc4c3c2)nc(-c2cccc3oc4ccccc4c23)n1 ; OB(O)c1cccc2oc3ccccc3c12 ; c1ccc2c(c1)ccc1ccc(-c3nc(-c4cccc5oc6ccccc6c45)nc(-c4cccc5oc6ccccc6c45)n3)cc12]

Trz13 (15 g, 32.8 mmol) and dibenzo[b,d]furan-1-ylboronic acid (7.3 g, 34.4 mmol) were added to 300 ml of THF, and the mixture was stirred and refluxed. Then, potassium carbonate (13.6 g, 98.3 mmol) was dissolved in 41 ml of water and added thereto, and the mixture was sufficiently stirred, and then bis(tri-tert-butylphosphine)palladium(0) (0.2 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 14.3 g of Compound 1-13 (Yield: 74%, MS: [M+H]+=590)

### Preparation Example 1-14

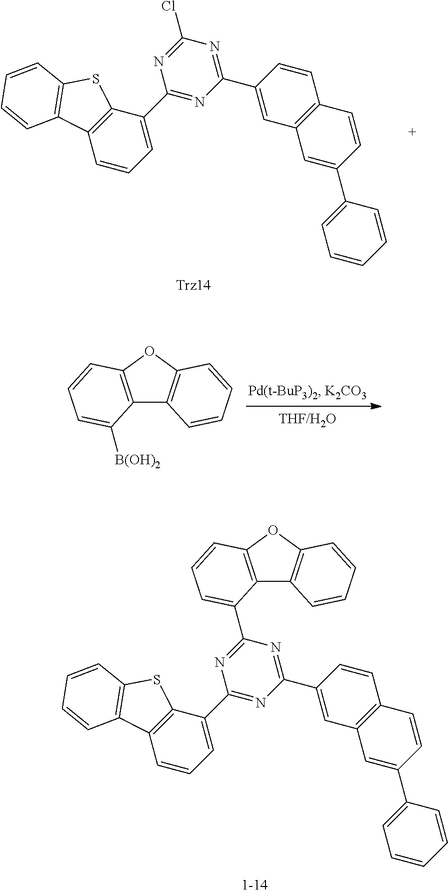 [SMILES: Clc1nc(-c2ccc3ccc(-c4ccccc4)cc3c2)nc(-c2cccc3c2sc2ccccc23)n1 ; OB(O)c1cccc2oc3ccccc3c12 ; c1ccc(-c2ccc3ccc(-c4nc(-c5cccc6c5sc5ccccc56)nc(-c5cccc6oc7ccccc7c56)n4)cc3c2)cc1]

Trz14 (15 g, 30 mmol) and dibenzo[b,d]furan-1-ylboronic acid (6.7 g, 31.5 mmol) were added to 300 ml of THF, and the mixture was stirred and refluxed. Then, potassium carbonate (12.4 g, 90 mmol) was dissolved in 37 ml of water and added thereto, and the mixture was sufficiently stirred, and then bis(tri-tert-butylphosphine)palladium(0) (0.2 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 14 g of Compound 1-14 (Yield: 74%, MS: [M+H]+=632)

### Preparation Example 1-15

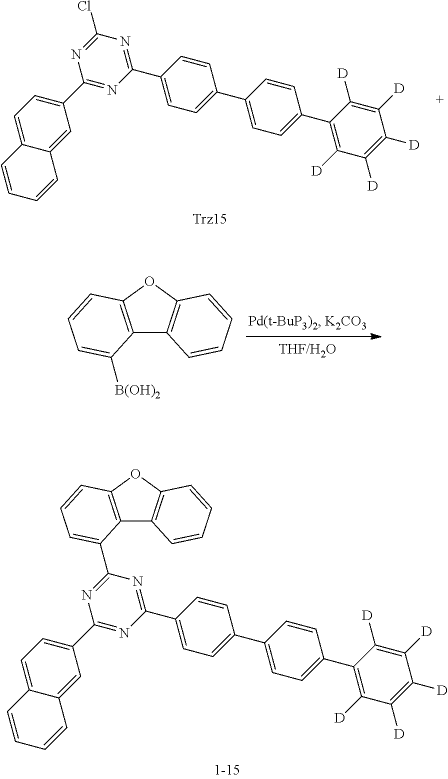 [SMILES: [2H]c1c([2H])c([2H])c(-c2ccc(-c3ccc(-c4nc(Cl)nc(-c5ccc6ccccc6c5)n4)cc3)cc2)c([2H])c1[2H] ; OB(O)c1cccc2oc3ccccc3c12 ; [2H]c1c([2H])c([2H])c(-c2ccc(-c3ccc(-c4nc(-c5ccc6ccccc6c5)nc(-c5cccc6oc7ccccc7c56)n4)cc3)cc2)c([2H])c1[2H]]

Trz15 (15 g, 31.6 mmol) and dibenzo[b,d]furan-1-ylboronic acid (7 g, 33.2 mmol) were added to 300 ml of THF, and the mixture was stirred and refluxed. Then, potassium carbonate (13.1 g, 94.7 mmol) was dissolved in 39 ml of water and added thereto, and the mixture was sufficiently stirred, and then bis(tri-tert-butylphosphine)palladium(0) (0.2 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 14.2 g of Compound 1-15 (Yield: 74%, MS: [M+H]+=607)

### Preparation Example 1-16

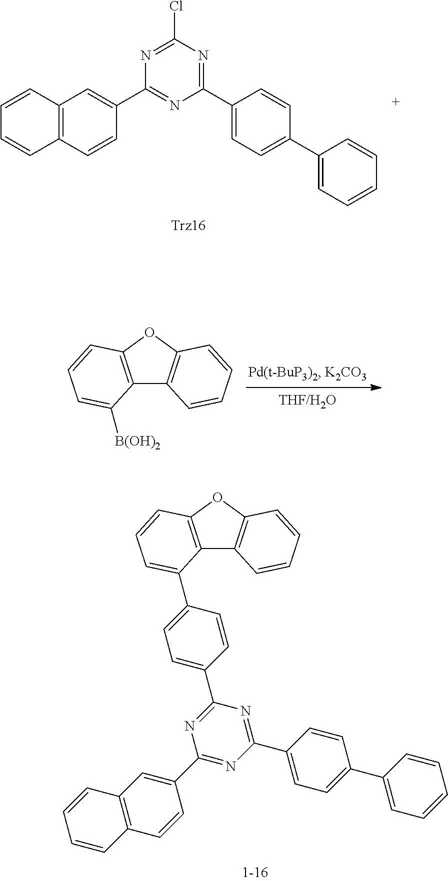 [SMILES: Clc1nc(-c2ccc(-c3ccccc3)cc2)nc(-c2ccc3ccccc3c2)n1 ; OB(O)c1cccc2oc3ccccc3c12 ; c1ccc(-c2ccc(-c3nc(-c4ccc(-c5cccc6oc7ccccc7c56)cc4)nc(-c4ccc5ccccc5c4)n3)cc2)cc1]

Trz16 (15 g, 31.9 mmol) and dibenzo[b,d]furan-1-ylboronic acid (7.1 g, 33.5 mmol) were added to 300 ml of THF, and the mixture was stirred and refluxed. Then, potassium carbonate (13.2 g, 95.8 mmol) was dissolved in 40 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.7 g of Compound 1-16 (Yield: 66%, MS: [M+H]+=602)

### Preparation Example 1-17

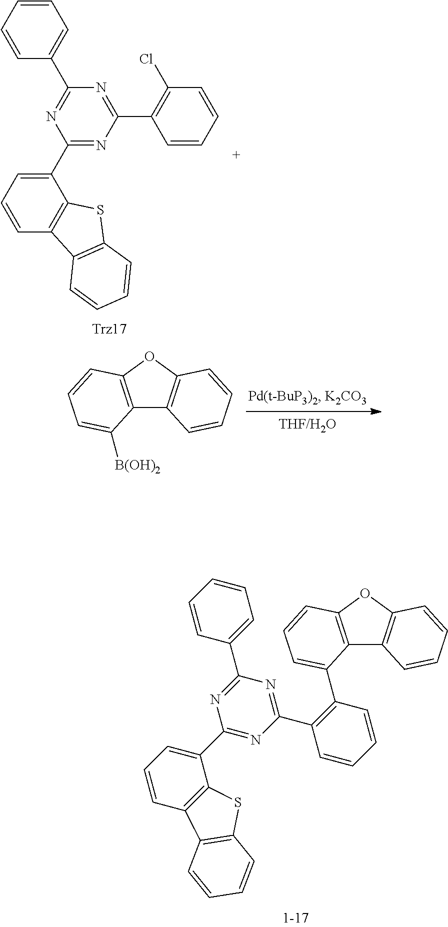 [SMILES: Clc1ccccc1-c1nc(-c2ccccc2)nc(-c2cccc3c2sc2ccccc23)n1 ; OB(O)c1cccc2oc3ccccc3c12 ; c1ccc(-c2nc(-c3ccccc3-c3cccc4oc5ccccc5c34)nc(-c3cccc4c3sc3ccccc34)n2)cc1]

Trz17 (15 g, 33.3 mmol) and dibenzo[b,d]furan-1-ylboronic acid (7.4 g, 35 mmol) were added to 300 ml of THF, and the mixture was stirred and refluxed. Then, potassium carbonate (13.8 g, 100 mmol) was dissolved in 41 ml of water and added thereto, and the mixture was sufficiently stirred, and then bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.9 g of Compound 1-17 (Yield: 72%, MS: [M+H]+=582)

### Preparation Example 1-18

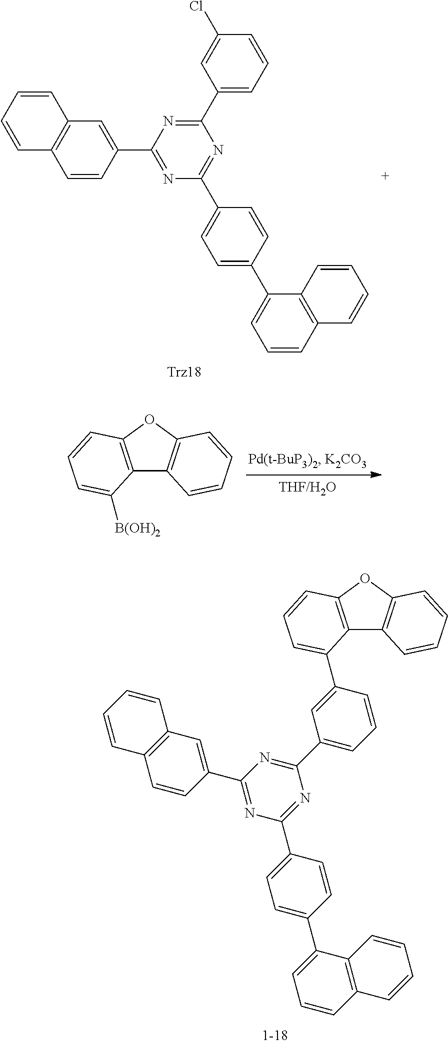 [SMILES: Clc1cccc(-c2nc(-c3ccc(-c4cccc5ccccc45)cc3)nc(-c3ccc4ccccc4c3)n2)c1 ; OB(O)c1cccc2oc3ccccc3c12 ; c1cc(-c2nc(-c3ccc(-c4cccc5ccccc45)cc3)nc(-c3ccc4ccccc4c3)n2)cc(-c2cccc3oc4ccccc4c23)c1]

Trz18 (15 g, 28.8 mmol) and dibenzo[b,d]furan-1-ylboronic acid (6.4 g, 30.3 mmol) were added to 300 ml of THF, and the mixture was stirred and refluxed. Then, potassium carbonate (12 g, 86.5 mmol) was dissolved in 36 ml of water and added thereto, and the mixture was sufficiently stirred, and then bis(tri-tert-butylphosphine)palladium(0) (0.1 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.3 g of Compound 1-18 (Yield: 71%, MS: [M+H]+=652)

### Preparation Example 1-19

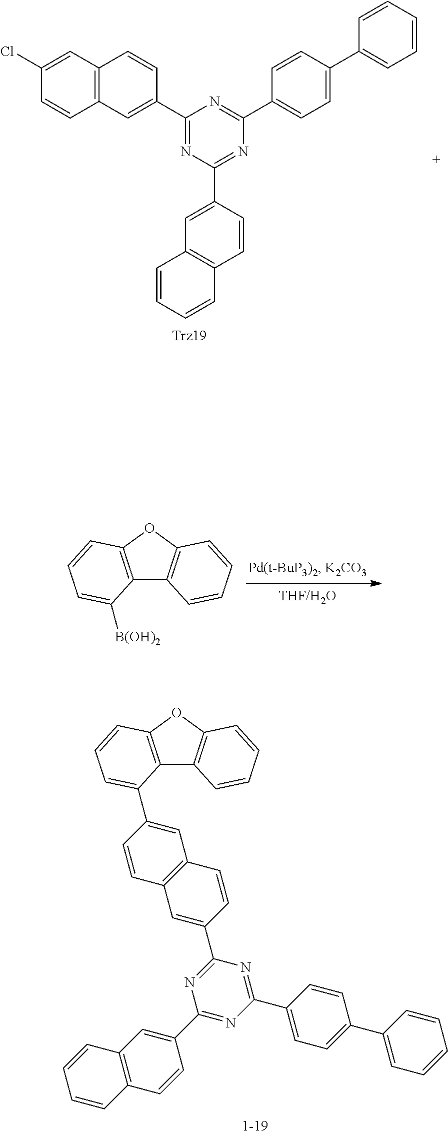 [SMILES: Clc1ccc2cc(-c3nc(-c4ccc(-c5ccccc5)cc4)nc(-c4ccc5ccccc5c4)n3)ccc2c1 ; OB(O)c1cccc2oc3ccccc3c12 ; c1ccc(-c2ccc(-c3nc(-c4ccc5ccccc5c4)nc(-c4ccc5cc(-c6cccc7oc8ccccc8c67)ccc5c4)n3)cc2)cc1]

Trz19 (15 g, 28.8 mmol) and dibenzo[b,d]furan-1-ylboronic acid (6.4 g, 30.3 mmol) were added to 300 ml of THF, and the mixture was stirred and refluxed. Then, potassium carbonate (12 g, 86.5 mmol) was dissolved in 36 ml of water and added thereto, and the mixture was sufficiently stirred, and then bis(tri-tert-butylphosphine)palladium(0) (0.1 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.7 g of Compound 1-19 (Yield: 73%, MS: [M+H]+=652)

### Preparation Example 1-20

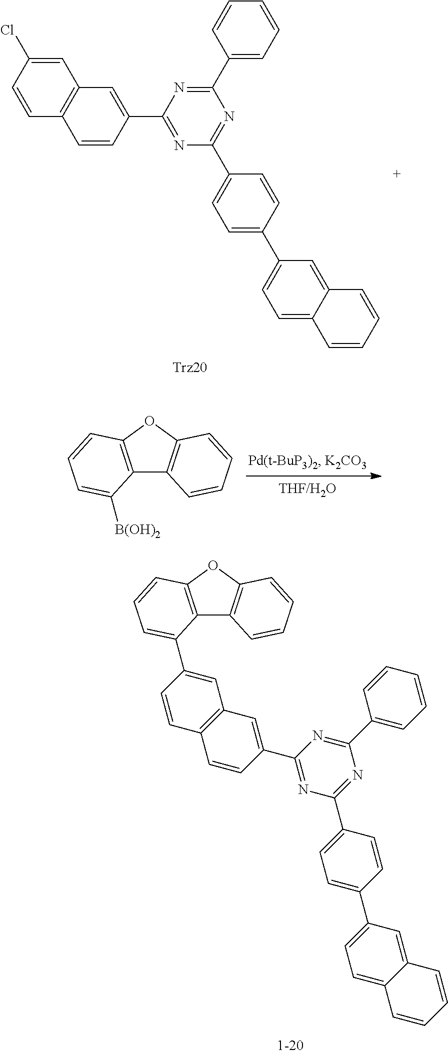 [SMILES: Clc1ccc2ccc(-c3nc(-c4ccccc4)nc(-c4ccc(-c5ccc6ccccc6c5)cc4)n3)cc2c1 ; OB(O)c1cccc2oc3ccccc3c12 ; c1ccc(-c2nc(-c3ccc(-c4ccc5ccccc5c4)cc3)nc(-c3ccc4ccc(-c5cccc6oc7ccccc7c56)cc4c3)n2)cc1]

Trz20 (15 g, 28.8 mmol) and dibenzo[b,d]furan-1-ylboronic acid (6.4 g, 30.3 mmol) were added to 300 ml of THF, and the mixture was stirred and refluxed. Then, potassium carbonate (12 g, 86.5 mmol) was dissolved in 36 ml of water and added thereto, and the mixture was sufficiently stirred, and then bis(tri-tert-butylphosphine)palladium(0) (0.1 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.6 g of Compound 1-20 (Yield: 67%, MS: [M+H]+=652)

### Preparation Example 1-21

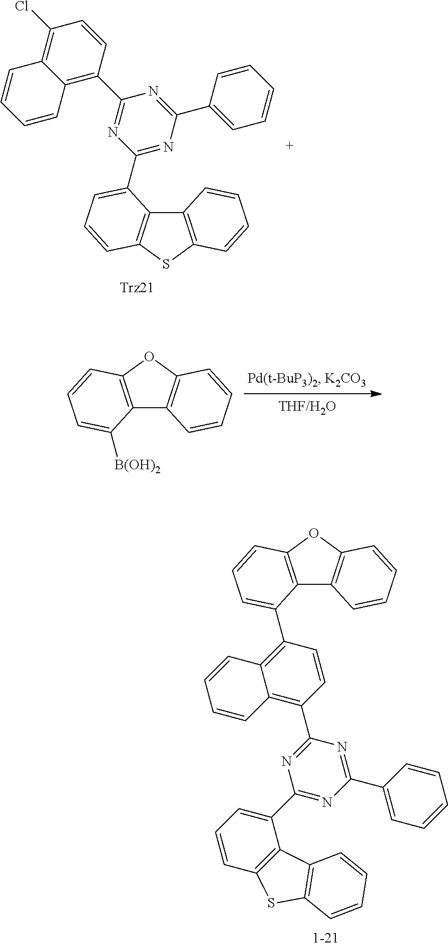 [SMILES: Clc1ccc(-c2nc(-c3ccccc3)nc(-c3cccc4sc5ccccc5c34)n2)c2ccccc12 ; OB(O)c1cccc2oc3ccccc3c12 ; c1ccc(-c2nc(-c3ccc(-c4cccc5oc6ccccc6c45)c4ccccc34)nc(-c3cccc4sc5ccccc5c34)n2)cc1]

Trz21 (15 g, 30 mmol) and dibenzo[b,d]furan-1-ylboronic acid (6.7 g, 31.5 mmol) were added to 300 ml of THF, and the mixture was stirred and refluxed. Then, potassium carbonate (12.4 g, 90 mmol) was dissolved in 37 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 14.2 g of Compound 1-21 (Yield: 75%, MS: [M+H]+=632)

### Preparation Example 1-22

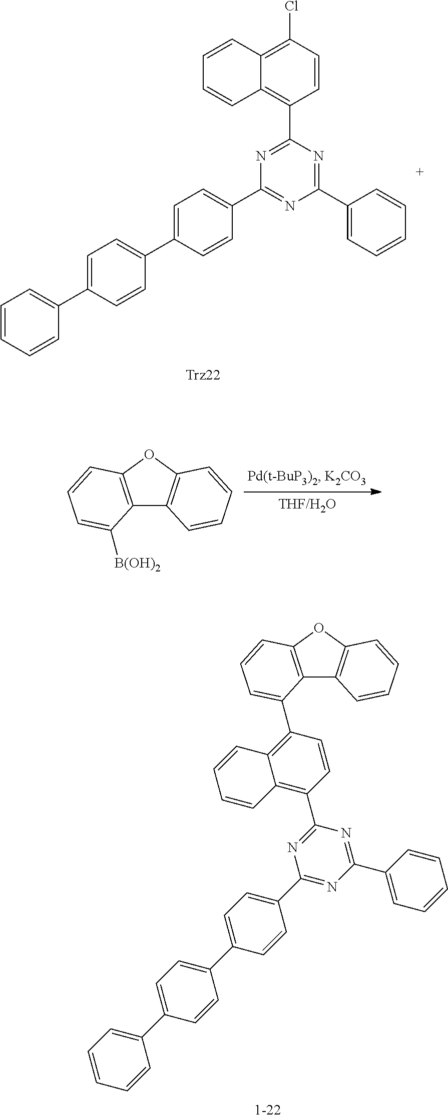 [SMILES: Clc1ccc(-c2nc(-c3ccccc3)nc(-c3ccc(-c4ccc(-c5ccccc5)cc4)cc3)n2)c2ccccc12 ; OB(O)c1cccc2oc3ccccc3c12 ; c1ccc(-c2ccc(-c3ccc(-c4nc(-c5ccccc5)nc(-c5ccc(-c6cccc7oc8ccccc8c67)c6ccccc56)n4)cc3)cc2)cc1]

Trz22 (15 g, 27.5 mmol) and dibenzo[b,d]furan-1-ylboronic acid (6.1 g, 28.8 mmol) were added to 300 ml of THF, and the mixture was stirred and refluxed. Then, potassium carbonate (11.4 g, 82.4 mmol) was dissolved in 34 ml of water and added thereto, and the mixture was sufficiently stirred, and then bis(tri-tert-butylphosphine)palladium(0) (0.1 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 14 g of Compound 1-22 (Yield: 75%, MS: [M+H]+=678)

### Preparation Example 1-23

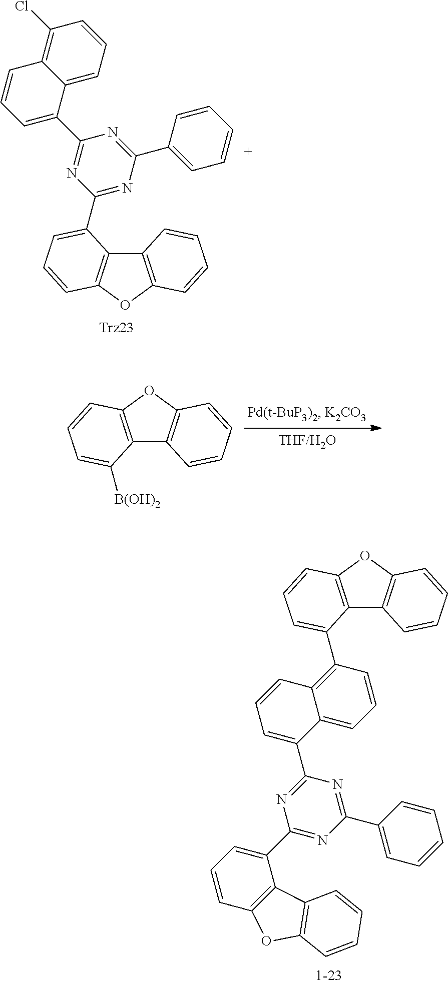 [SMILES: Clc1cccc2c(-c3nc(-c4ccccc4)nc(-c4cccc5oc6ccccc6c45)n3)cccc12 ; OB(O)c1cccc2oc3ccccc3c12 ; c1ccc(-c2nc(-c3cccc4c(-c5cccc6oc7ccccc7c56)cccc34)nc(-c3cccc4oc5ccccc5c34)n2)cc1]

Trz23 (15 g, 31 mmol) and dibenzo[b,d]furan-1-ylboronic acid (6.9 g, 32.5 mmol) were added to 300 ml of THF, and the mixture was stirred and refluxed. Then, potassium carbonate (12.9 g, 93 mmol) was dissolved in 39 ml of water and added thereto, and the mixture was sufficiently stirred, and then bis(tri-tert-butylphosphine)palladium(0) (0.2 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13 g of Compound 1-23 (Yield: 68%, MS: [M+H]+=616)

### Preparation Example 1-24

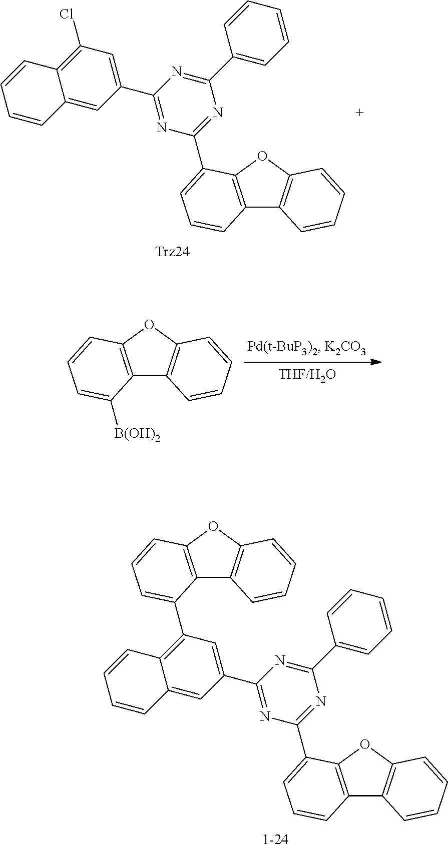 [SMILES: Clc1cc(-c2nc(-c3ccccc3)nc(-c3cccc4c3oc3ccccc34)n2)cc2ccccc12 ; OB(O)c1cccc2oc3ccccc3c12 ; c1ccc(-c2nc(-c3cc(-c4cccc5oc6ccccc6c45)c4ccccc4c3)nc(-c3cccc4c3oc3ccccc34)n2)cc1]

Trz24 (15 g, 31 mmol) and dibenzo[b,d]furan-1-ylboronic acid (6.9 g, 32.5 mmol) were added to 300 ml of THF, and the mixture was stirred and refluxed. Then, potassium carbonate (12.9 g, 93 mmol) was dissolved in 39 ml of water and added thereto, and the mixture was sufficiently stirred, and then bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.3 g of Compound 1-24 (Yield: 70%, MS: [M+H]+=616)

### Preparation Example 1-25

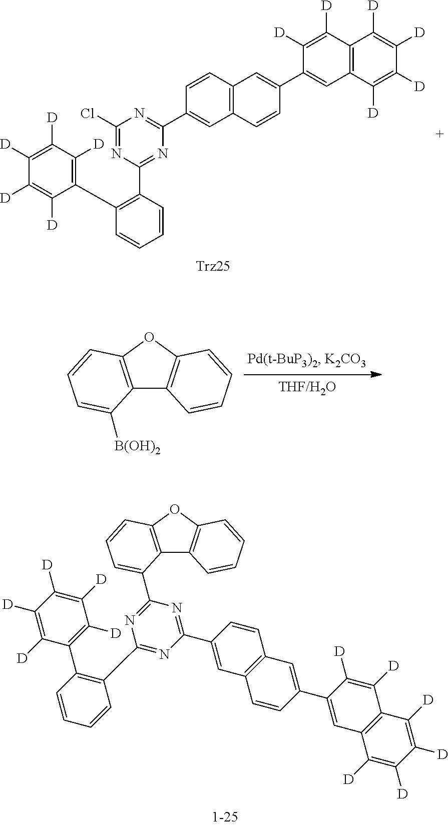 [SMILES: [2H]c1c([2H])c([2H])c(-c2ccccc2-c2nc(Cl)nc(-c3ccc4cc(-c5cc6c([2H])c([2H])c([2H])c([2H])c6c([2H])c5[2H])ccc4c3)n2)c([2H])c1[2H] ; OB(O)c1cccc2oc3ccccc3c12 ; [2H]c1c([2H])c([2H])c(-c2ccccc2-c2nc(-c3ccc4cc(-c5cc6c([2H])c([2H])c([2H])c([2H])c6c([2H])c5[2H])ccc4c3)nc(-c3cccc4oc5ccccc5c34)n2)c([2H])c1[2H]]

Trz25 (15 g, 28.2 mmol) and dibenzo[b,d]furan-1-ylboronic acid (6.3 g, 29.7 mmol) were added to 300 ml of THF, and the mixture was stirred and refluxed. Then, potassium carbonate (11.7 g, 84.7 mmol) was dissolved in 35 ml of water and added thereto, and the mixture was sufficiently stirred, and then bis(tri-tert-butylphosphine)palladium(0) (0.1 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.9 g of Compound 1-25 (Yield: 69%, MS: [M+H]+=663)

### Preparation Example 1-26

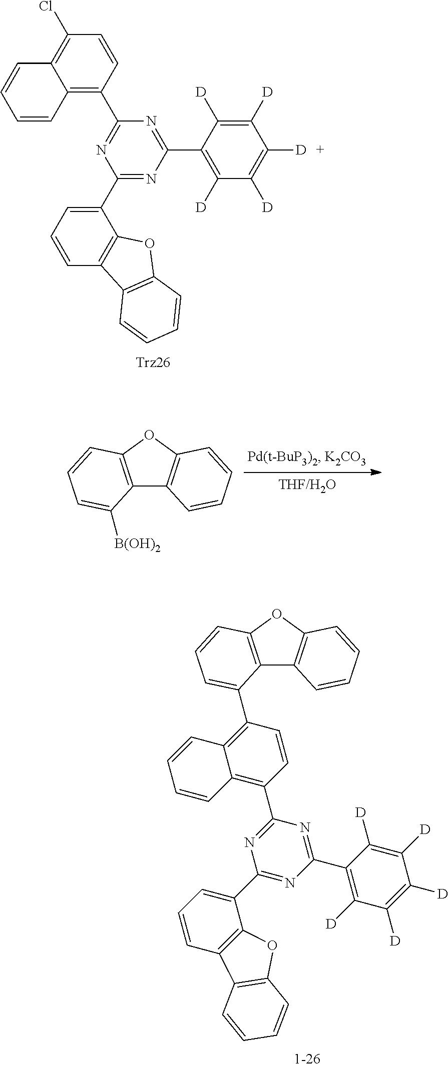 [SMILES: [2H]c1c([2H])c([2H])c(-c2nc(-c3ccc(Cl)c4ccccc34)nc(-c3cccc4c3oc3ccccc34)n2)c([2H])c1[2H] ; OB(O)c1cccc2oc3ccccc3c12 ; [2H]c1c([2H])c([2H])c(-c2nc(-c3ccc(-c4cccc5oc6ccccc6c45)c4ccccc34)nc(-c3cccc4c3oc3ccccc34)n2)c([2H])c1[2H]]

Trz26 (15 g, 30.7 mmol) and dibenzo[b,d]furan-1-ylboronic acid (6.8 g, 32.2 mmol) were added to 300 ml of THF, and the mixture was stirred and refluxed. Then, potassium carbonate (12.7 g, 92 mmol) was dissolved in 38 ml of water and added thereto, and the mixture was sufficiently stirred, and then bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 14.3 g of Compound 1-26 (Yield: 75%, MS: [M+H]+=621)

### Preparation Example 1-27

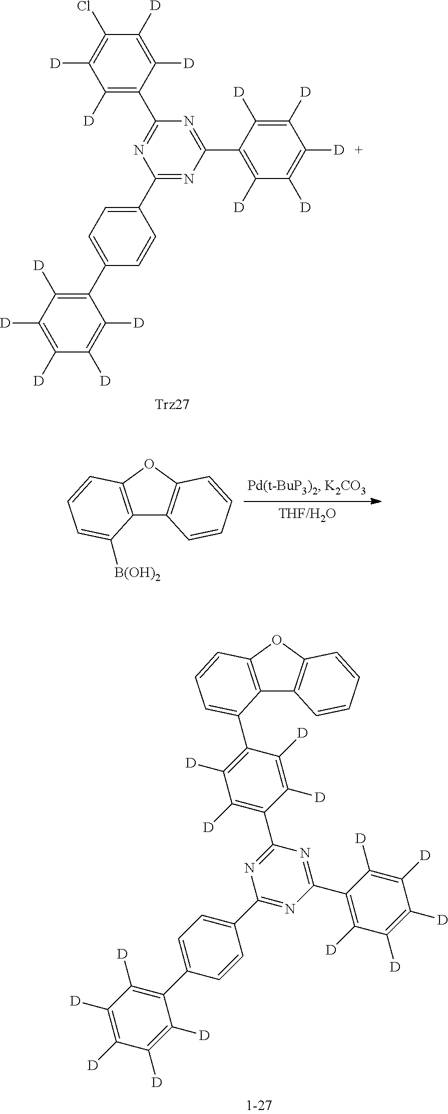 [SMILES: [2H]c1c([2H])c([2H])c(-c2ccc(-c3nc(-c4c([2H])c([2H])c([2H])c([2H])c4[2H])nc(-c4c([2H])c([2H])c(Cl)c([2H])c4[2H])n3)cc2)c([2H])c1[2H] ; OB(O)c1cccc2oc3ccccc3c12 ; [2H]c1c([2H])c([2H])c(-c2ccc(-c3nc(-c4c([2H])c([2H])c([2H])c([2H])c4[2H])nc(-c4c([2H])c([2H])c(-c5cccc6oc7ccccc7c56)c([2H])c4[2H])n3)cc2)c([2H])c1[2H]]

Trz27 (15 g, 34.6 mmol) and dibenzo[b,d]furan-1-ylboronic acid (7.7 g, 36.3 mmol) were added to 300 ml of THF, and the mixture was stirred and refluxed. Then, potassium carbonate (14.3 g, 103.7 mmol) was dissolved in 43 ml of water and added thereto, and the mixture was sufficiently stirred, and then bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.9 g of Compound 1-27 (Yield: 71%, MS: [M+H]+=566)

### Preparation Example 1-28

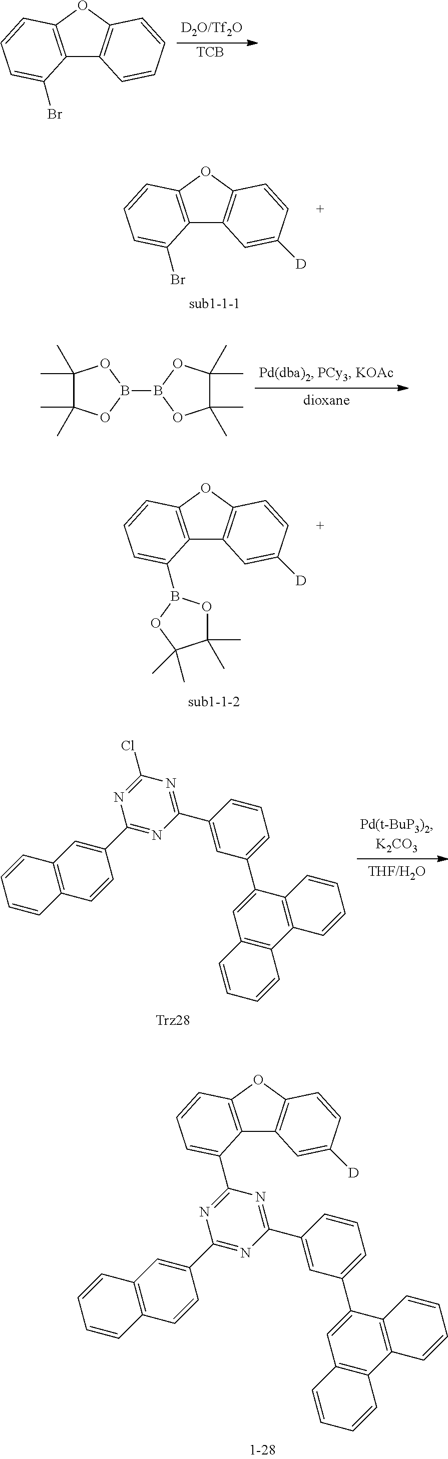

Trifluoromethanesulfonic anhydride (24 g, 85 mmol) and deuterium oxide (8.5 g, 424.9 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After the reaction for 5 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.7 g of sub1-1-1. (Yield: 38%, MS: [M+H]⁺=248)

sub1-1-1 (15 g, 60.5 mmol) and bis(pinacolato)diboron (16.9 g, 66.5 mmol) were added to 300 ml of 1,4-dioxane, and the mixture was stirred and refluxed. Then, potassium acetate (8.9 g, 90.7 mmol) was added thereto, and the mixture was sufficiently stirred, and then bis(dibenzylideneacetone)palladium(0) (1 g, 1.8 mmol) and tricyclohexylphosphine (1 g, 3.6 mmol) were added. After the reaction for 6 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.4 g of sub1-1-2. (Yield: 75%, MS: [M+H]+=296)

sub1-1-2 (15 g, 50.8 mmol) and Trz28 (26.4 g, 53.4 mmol) were added to 300 ml of THF, and the mixture was stirred and refluxed. Then, potassium carbonate (21.1 g, 152.5 mmol) was dissolved in 63 ml of water and added thereto, and the mixture was sufficiently stirred, and then bis(tri-tert-butylphosphine)palladium(0) (0.3 g, 0.5 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 21 g of Compound 1-28. (Yield: 66%, MS: [M+H]+=627)

### Preparation Example 1-29

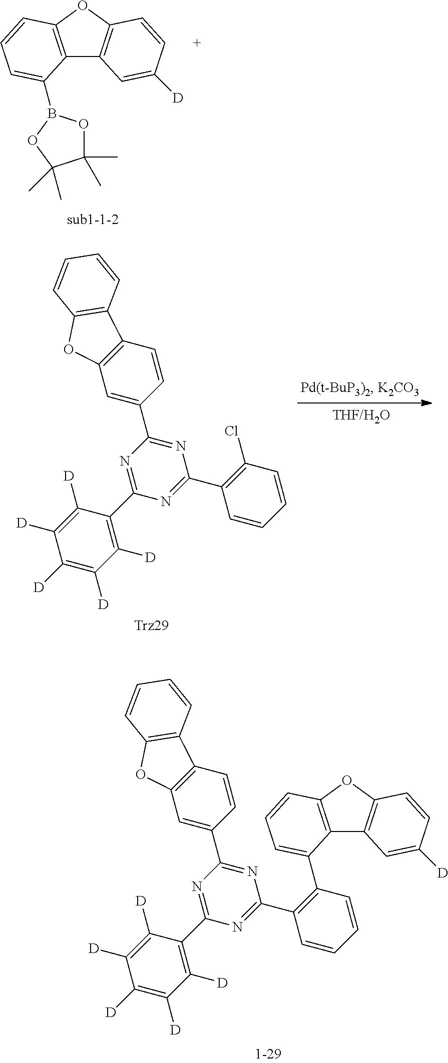 [SMILES: [2H]c1ccc2oc3cccc(B4OC(C)(C)C(C)(C)O4)c3c2c1 ; [2H]c1c([2H])c([2H])c(-c2nc(-c3ccc4c(c3)oc3ccccc34)nc(-c3ccccc3Cl)n2)c([2H])c1[2H] ; [2H]c1ccc2oc3cccc(-c4ccccc4-c4nc(-c5ccc6c(c5)oc5ccccc56)nc(-c5c([2H])c([2H])c([2H])c([2H])c5[2H])n4)c3c2c1]

sub1-1-2 (15 g, 50.8 mmol) and Trz29 (23.4 g, 53.4 mmol) were added to 300 ml of THF, and the mixture was stirred and refluxed. Then, potassium carbonate (21.1 g, 152.5 mmol) was dissolved in 63 ml of water and added thereto, and the mixture was sufficiently stirred, and then bis(tri-tert-butylphosphine)palladium(0) (0.3 g, 0.5 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 19.4 g of Compound 1-29. (Yield: 67%, MS: [M+H]+=572)

### Preparation Example 1-30

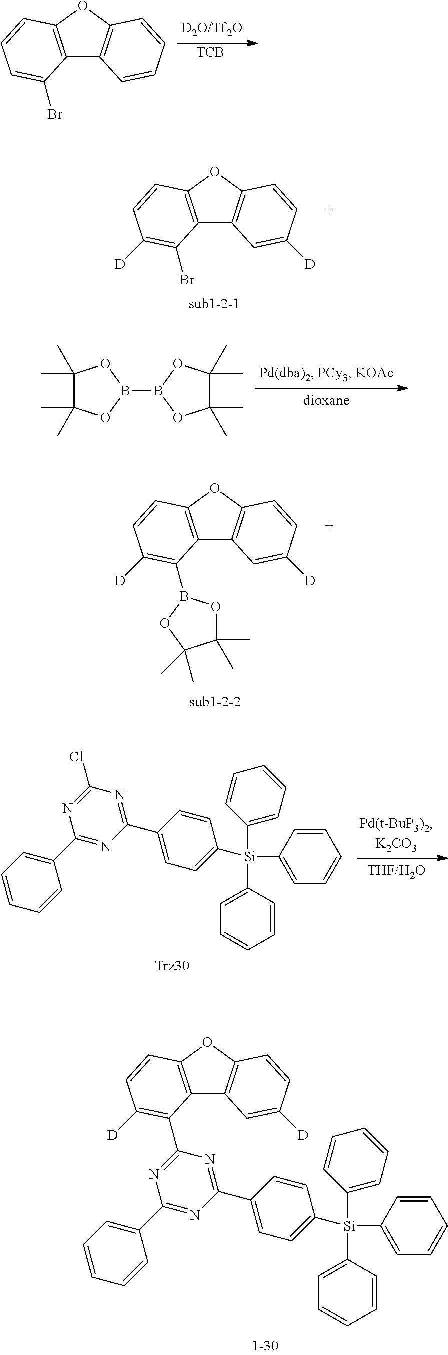

Trifluoromethanesulfonic anhydride (48 g, 170 mmol) and deuterium oxide (17 g, 849.9 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6 g of sub1-2-1. (Yield: 40%, MS: [M+H]+=249)

sub1-2-1 (15 g, 60.2 mmol) and bis(pinacolato)diboron (16.8 g, 66.2 mmol) were added to 300 ml of 1,4-dioxane, and the mixture was stirred and refluxed. Then, potassium acetate (8.9 g, 90.3 mmol) was added thereto, and the mixture was sufficiently stirred, and then bis(dibenzylideneacetone)palladium(0) (1 g, 1.8 mmol) and tricyclohexylphosphine (1 g, 3.6 mmol) were added. After the reaction for 4 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.5 g of sub1-2-2. (Yield: 70%, MS: [M+H]+=297)

sub1-2-2 (15 g, 50.6 mmol) and Trz30 (28 g, 53.2 mmol) were added to 300 ml of THF, and the mixture was stirred and refluxed. Then, potassium carbonate (21 g, 151.9 mmol) was dissolved in 63 ml of water and added thereto, and the mixture was sufficiently stirred, and then bis(tri-tert-butylphosphine)palladium(0) (0.3 g, 0.5 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 23.4 g of Compound 1-30. (Yield: 70%, MS: [M+H]+=660)

### Preparation Example 1-31

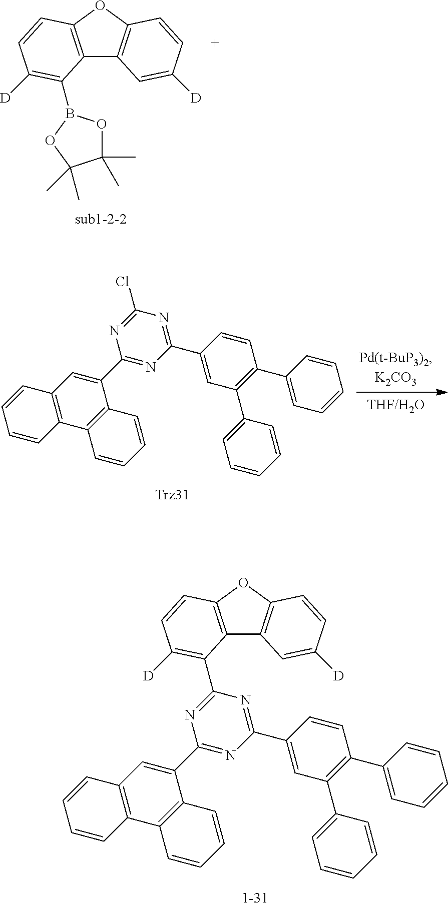 [SMILES: [2H]c1ccc2oc3ccc([2H])c(B4OC(C)(C)C(C)(C)O4)c3c2c1 ; Clc1nc(-c2ccc(-c3ccccc3)c(-c3ccccc3)c2)nc(-c2cc3ccccc3c3ccccc23)n1 ; [2H]c1ccc2oc3ccc([2H])c(-c4nc(-c5ccc(-c6ccccc6)c(-c6ccccc6)c5)nc(-c5cc6ccccc6c6ccccc56)n4)c3c2c1]

sub1-2-2 (15 g, 50.6 mmol) and Trz31 (21.9 g, 53.2 mmol) were added to 300 ml of THF, and the mixture was stirred and refluxed. Then, potassium carbonate (21 g, 151.9 mmol) was dissolved in 63 ml of water and added thereto, and the mixture was sufficiently stirred, and then bis(tri-tert-butylphosphine)palladium(0) (0.3 g, 0.5 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 22.5 g of Compound 1-31. (Yield: 68%, MS: [M+H]+=654)

### Preparation Example 1-32

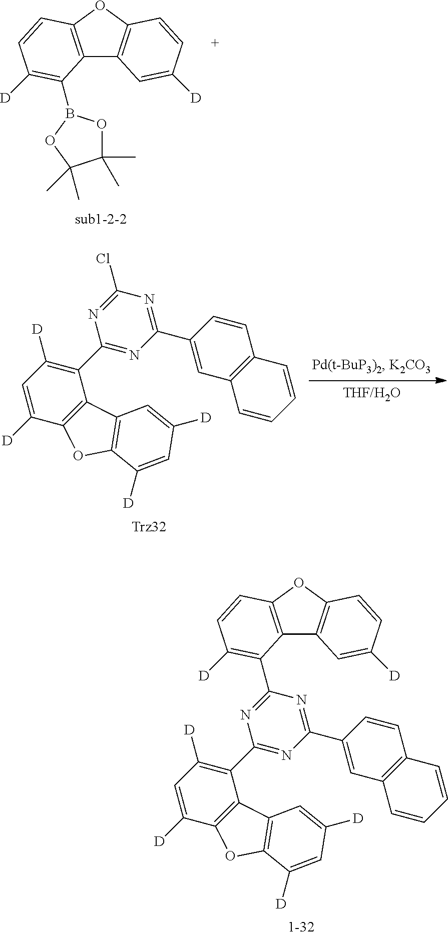 [SMILES: [2H]c1ccc2oc3ccc([2H])c(B4OC(C)(C)C(C)(C)O4)c3c2c1 ; [2H]c1cc([2H])c2oc3c([2H])cc([2H])c(-c4nc(Cl)nc(-c5ccc6ccccc6c5)n4)c3c2c1 ; [2H]c1ccc2oc3ccc([2H])c(-c4nc(-c5ccc6ccccc6c5)nc(-c5c([2H])cc([2H])c6oc7c([2H])cc([2H])cc7c56)n4)c3c2c1]

sub1-2-2 (15 g, 50.6 mmol) and Trz32 (21.9 g, 53.2 mmol) were added to 300 ml of THF, and the mixture was stirred and refluxed. Then, potassium carbonate (21 g, 151.9 mmol) was dissolved in 63 ml of water and added thereto, and the mixture was sufficiently stirred, and then bis(tri-tert-butylphosphine)palladium(0) (0.3 g, 0.5 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 17.9 g of Compound 1-32. (Yield: 65%, MS: [M+H]+=546)

### Preparation Example 1-33

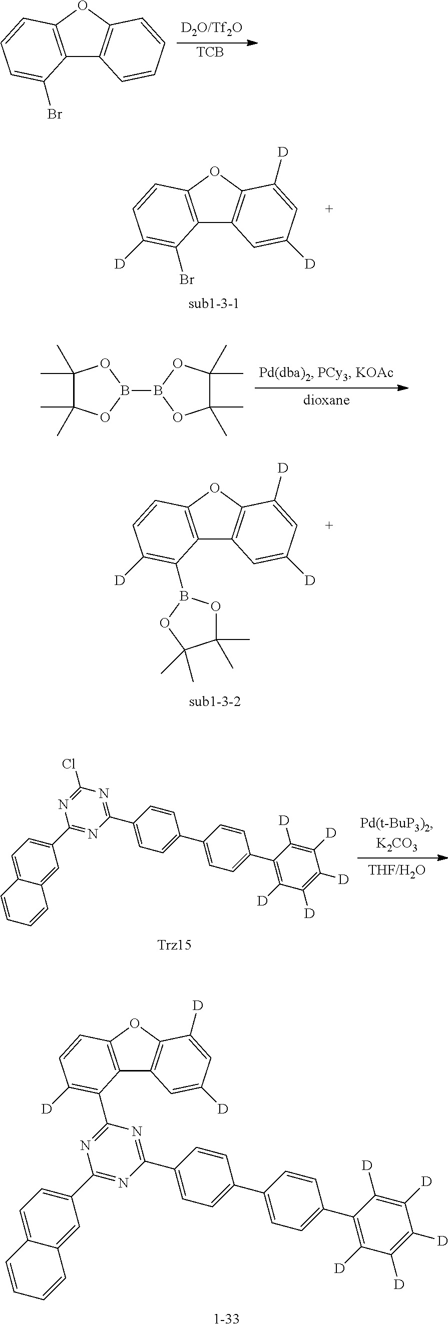

Trifluoromethanesulfonic anhydride (71.9 g, 255 mmol) and deuterium oxide (25.5 g, 1274.8 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After the reaction for 14 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.3 g of sub1-3-1. (Yield: 42%, MS: [M+H]+=250)

sub1-3-1 (15 g, 60 mmol) and bis(pinacolato)diboron (16.8 g, 66 mmol) were added to 300 ml of 1,4-dioxane, and the mixture was stirred and refluxed. Then, potassium acetate (8.8 g, 90 mmol) was added thereto, and the mixture was sufficiently stirred, and then bis(dibenzylideneacetone)palladium(0) (1 g, 1.8 mmol) and tricyclohexylphosphine (1 g, 3.6 mmol) were added. After the reaction for 6 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.4 g of sub1-3-2. (Yield: 64%, MS: [M+H]+=298)

sub1-3-2 (15 g, 50.5 mmol) and Trz15 (25.2 g, 53 mmol) were added to 300 ml of THF, and the mixture was stirred and refluxed. Then, potassium carbonate (20.9 g, 151.4 mmol) was dissolved in 63 ml of water and added thereto, and the mixture was sufficiently stirred, and then bis(tri-tert-butylphosphine)palladium(0) (0.3 g, 0.5 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 23.1 g of Compound 1-33. (Yield: 75%, MS: [M+H]+=610)

### Preparation Example 1-34

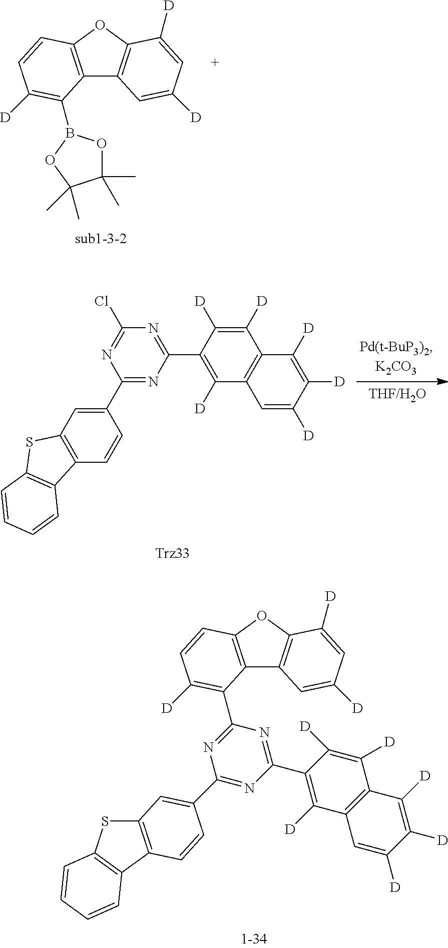 [SMILES: [2H]c1cc([2H])c2oc3ccc([2H])c(B4OC(C)(C)C(C)(C)O4)c3c2c1 ; [2H]c1cc2c([2H])c(-c3nc(Cl)nc(-c4ccc5c(c4)sc4ccccc45)n3)c([2H])c([2H])c2c([2H])c1[2H] ; [2H]c1cc([2H])c2oc3ccc([2H])c(-c4nc(-c5ccc6c(c5)sc5ccccc56)nc(-c5c([2H])c([2H])c6c([2H])c([2H])c([2H])cc6c5[2H])n4)c3c2c1]

sub1-3-2 (15 g, 50.5 mmol) and Trz33 (22.8 g, 53 mmol) were added to 300 ml of THF, and the mixture was stirred and refluxed. Then, potassium carbonate (20.9 g, 151.4 mmol) was dissolved in 63 ml of water and added thereto, and the mixture was sufficiently stirred, and then bis(tri-tert-butylphosphine)palladium(0) (0.3 g, 0.5 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 18.5 g of Compound 1-34. (Yield: 65%, MS: [M+H]+=565)

### Preparation Example 1-35

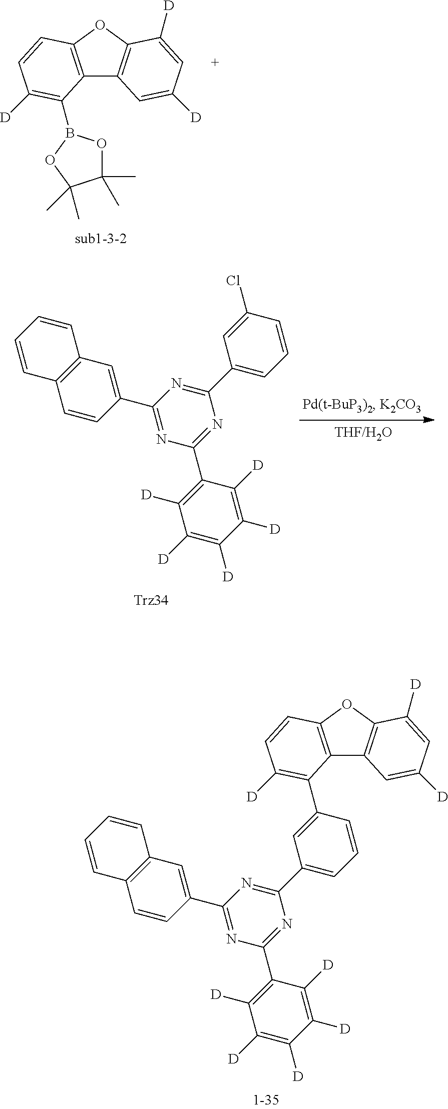 [SMILES: [2H]c1cc([2H])c2oc3ccc([2H])c(B4OC(C)(C)C(C)(C)O4)c3c2c1 ; [2H]c1c([2H])c([2H])c(-c2nc(-c3cccc(Cl)c3)nc(-c3ccc4ccccc4c3)n2)c([2H])c1[2H] ; [2H]c1cc([2H])c2oc3ccc([2H])c(-c4cccc(-c5nc(-c6ccc7ccccc7c6)nc(-c6c([2H])c([2H])c([2H])c([2H])c6[2H])n5)c4)c3c2c1]

sub1-3-2 (15 g, 50.5 mmol) and Trz34 (21.1 g, 53 mmol) were added to 300 ml of THF, and the mixture was stirred and refluxed. Then, potassium carbonate (20.9 g, 151.4 mmol) was dissolved in 63 ml of water and added thereto, and the mixture was sufficiently stirred, and then bis(tri-tert-butylphosphine)palladium(0) (0.3 g, 0.5 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 17.8 g of Compound 1-35. (Yield: 66%, MS: [M+H]+=534)

### Preparation Example 1-36

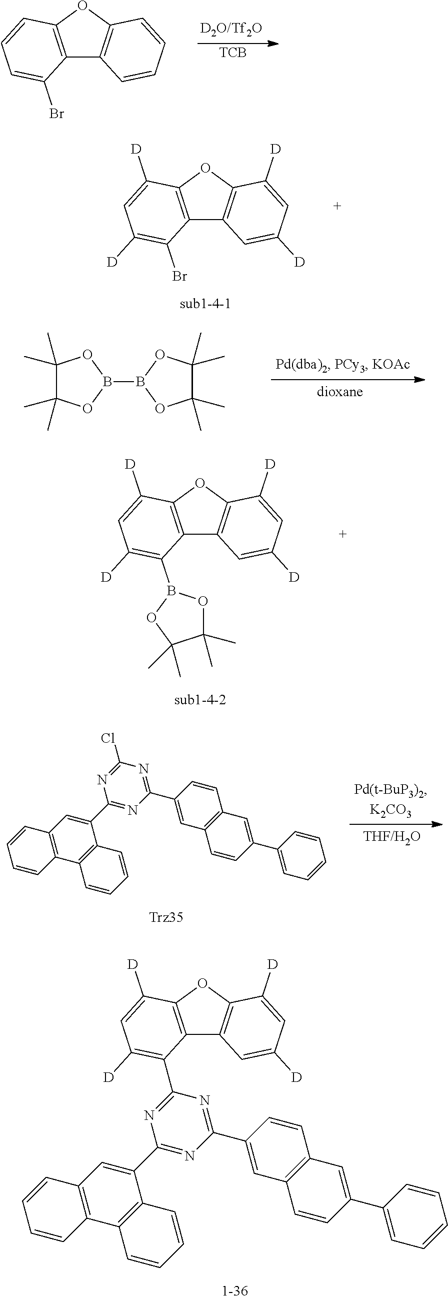

Trifluoromethanesulfonic anhydride (95.9 g, 340 mmol) and deuterium oxide (34 g, 1699.8 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After the reaction for 20 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.6 g of sub1-4-1. (Yield: 37%, MS: [M+H]+=251)

sub1-4-1 (15 g, 59.7 mmol) and bis(pinacolato)diboron (16.7 g, 65.7 mmol) were added to 300 ml of 1,4-dioxane, and the mixture was stirred and refluxed. Then, potassium acetate (8.8 g, 89.6 mmol) was added thereto, and the mixture was sufficiently stirred, and then bis(dibenzylideneacetone)palladium(0) (1 g, 1.8 mmol) and tricyclohexylphosphine (1 g, 3.6 mmol) were added. After the reaction for 5 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.5 g of sub1-4-2. (Yield: 70%, MS: [M+H]+=299)

sub1-4-2 (15 g, 50.3 mmol) and Trz35 (26.1 g, 52.8 mmol) were added to 300 ml of THF, and the mixture was stirred and refluxed. Then, potassium carbonate (20.9 g, 150.9 mmol) was dissolved in 63 ml of water and added thereto, and the mixture was sufficiently stirred, and then bis(tri-tert-butylphosphine)palladium(0) (0.3 g, 0.5 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 21.5 g of Compound 1-36. (Yield: 68%, MS: [M+H]+=631)

### Preparation Example 1-37

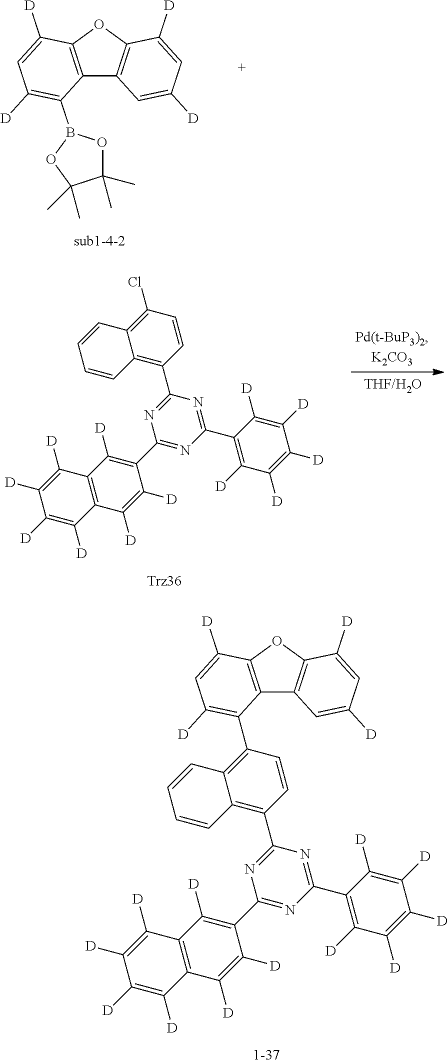 [SMILES: [2H]c1cc([2H])c2oc3c([2H])cc([2H])c(B4OC(C)(C)C(C)(C)O4)c3c2c1 ; [2H]c1c([2H])c([2H])c(-c2nc(-c3c([2H])c([2H])c4c([2H])c([2H])c([2H])c([2H])c4c3[2H])nc(-c3ccc(Cl)c4ccccc34)n2)c([2H])c1[2H] ; [2H]c1cc([2H])c2oc3c([2H])cc([2H])c(-c4ccc(-c5nc(-c6c([2H])c([2H])c([2H])c([2H])c6[2H])nc(-c6c([2H])c([2H])c7c([2H])c([2H])c([2H])c([2H])c7c6[2H])n5)c5ccccc45)c3c2c1]

sub1-4-2 (15 g, 50.3 mmol) and Trz36 (24.1 g, 52.8 mmol) were added to 300 ml of THF, and the mixture was stirred and refluxed. Then, potassium carbonate (20.9 g, 150.9 mmol) was dissolved in 63 ml of water and added thereto, and the mixture was sufficiently stirred, and then bis(tri-tert-butylphosphine)palladium(0) (0.3 g, 0.5 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 20.2 g of Compound 1-37. (Yield: 68%, MS: [M+H]+=592)

### Preparation Example 1-38

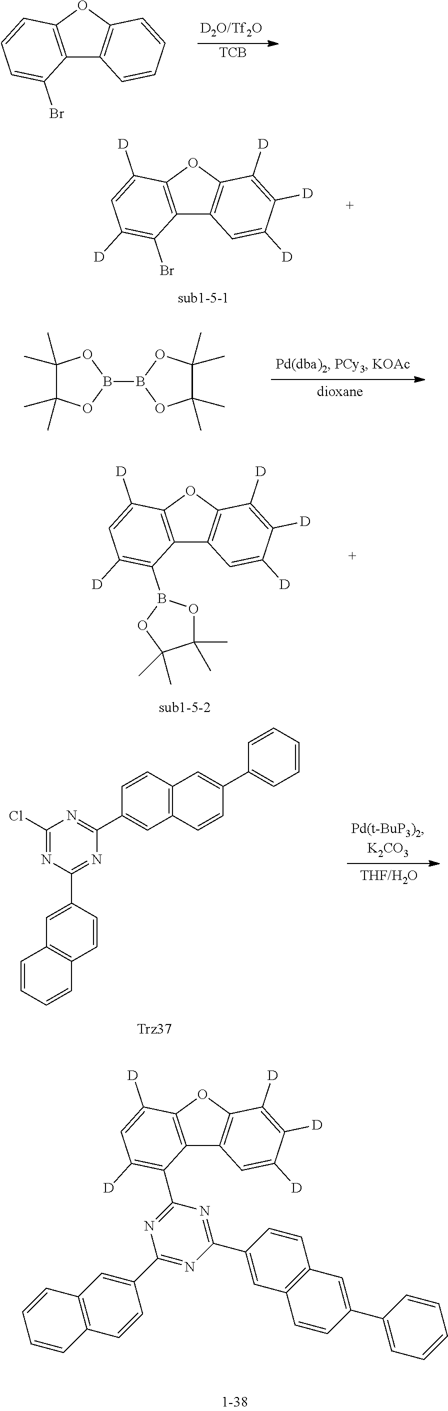

Trifluoromethanesulfonic anhydride (119.9 g, 424.9 mmol) and deuterium oxide (42.6 g, 2124.7 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After the reaction for 24 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.9 g of sub1-5-1. (Yield: 39%, MS: [M+H]+=252)

sub1-5-1 (15 g, 59.5 mmol) and bis(pinacolato)diboron (16.6 g, 65.4 mmol) were added to 300 ml of 1,4-dioxane, and the mixture was stirred and refluxed. Then, potassium acetate (8.8 g, 89.2 mmol) was added thereto, and the mixture was sufficiently stirred, and then bis(dibenzylideneacetone)palladium(0) (1 g, 1.8 mmol) and tricyclohexylphosphine (1 g, 3.6 mmol) were added. After the reaction for 4 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.2 g of sub1-5-2. (Yield: 63%, MS: [M+H]+=300)

sub1-5-2 (15 g, 50.1 mmol) and Trz37 (23.4 g, 52.6 mmol) were added to 300 ml of THF, and the mixture was stirred and refluxed. Then, potassium carbonate (20.8 g, 150.4 mmol) was dissolved in 62 ml of water and added thereto, and the mixture was sufficiently stirred, and then bis(tri-tert-butylphosphine)palladium(0) (0.3 g, 0.5 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 20.1 g of Compound 1-38. (Yield: 69%, MS: [M+H]+=581)

### Preparation Example 1-39

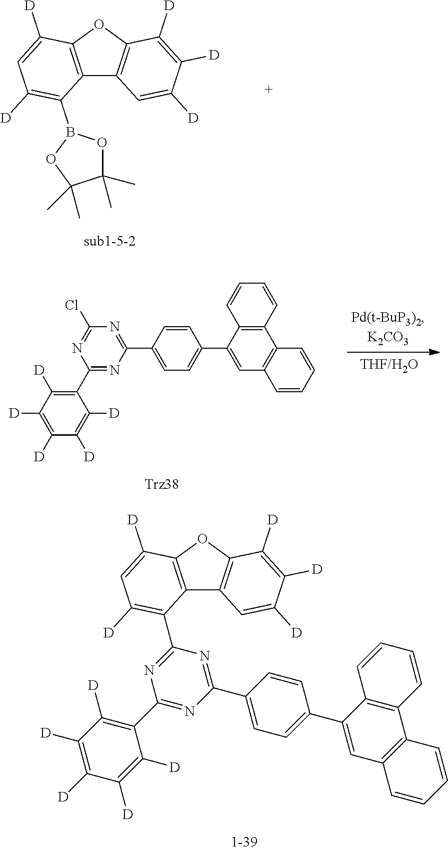 [SMILES: [2H]c1cc2c(oc3c([2H])cc([2H])c(B4OC(C)(C)C(C)(C)O4)c32)c([2H])c1[2H] ; [2H]c1c([2H])c([2H])c(-c2nc(Cl)nc(-c3ccc(-c4cc5ccccc5c5ccccc45)cc3)n2)c([2H])c1[2H] ; [2H]c1cc2c(oc3c([2H])cc([2H])c(-c4nc(-c5ccc(-c6cc7ccccc7c7ccccc67)cc5)nc(-c5c([2H])c([2H])c([2H])c([2H])c5[2H])n4)c32)c([2H])c1[2H]]

sub1-5-2 (15 g, 50.1 mmol) and Trz38 (23.6 g, 52.6 mmol) were added to 300 ml of THF, and the mixture was stirred and refluxed. Then, potassium carbonate (20.8 g, 150.4 mmol) was dissolved in 62 ml of water and added thereto, and the mixture was sufficiently stirred, and then bis(tri-tert-butylphosphine)palladium(0) (0.3 g, 0.5 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 20.2 g of Compound 1-39. (Yield: 69%, MS: [M+H]+=586)

### Preparation Example 1-40

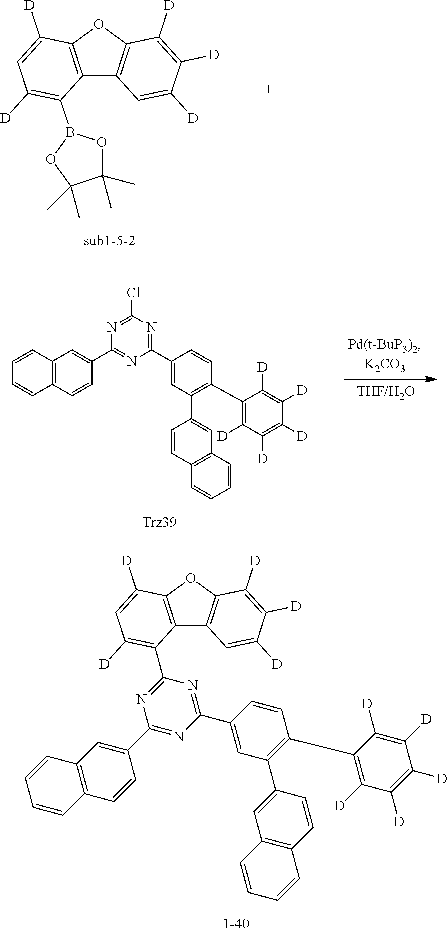 [SMILES: [2H]c1cc2c(oc3c([2H])cc([2H])c(B4OC(C)(C)C(C)(C)O4)c32)c([2H])c1[2H] ; [2H]c1c([2H])c([2H])c(-c2ccc(-c3nc(Cl)nc(-c4ccc5ccccc5c4)n3)cc2-c2ccc3ccccc3c2)c([2H])c1[2H] ; [2H]c1cc2c(oc3c([2H])cc([2H])c(-c4nc(-c5ccc(-c6c([2H])c([2H])c([2H])c([2H])c6[2H])c(-c6ccc7ccccc7c6)c5)nc(-c5ccc6ccccc6c5)n4)c32)c([2H])c1[2H]]

sub1-5-2 (15 g, 50.1 mmol) and Trz39 (27.6 g, 52.6 mmol) were added to 300 ml of THF, and the mixture was stirred and refluxed. Then, potassium carbonate (20.8 g, 150.4 mmol) was dissolved in 62 ml of water and added thereto, and the mixture was sufficiently stirred, and then bis(tri-tert-butylphosphine)palladium(0) (0.3 g, 0.5 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 22.5 g of Compound 1-40. (Yield: 68%, MS: [M+H]+=662)

### Preparation Example 1-41

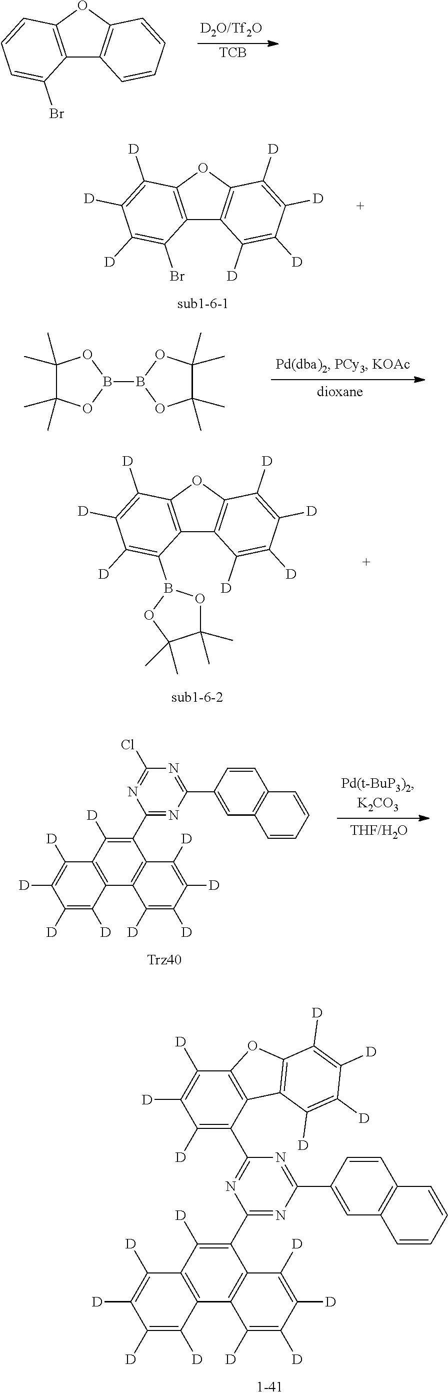

Trifluoromethanesulfonic anhydride (167.8 g, 594.9 mmol) and deuterium oxide (59.6 g, 2974.6 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After the reaction for 36 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.1 g of sub1-6-1. (Yield: 40%, MS: [M+H]+=254)

sub1-6-1 (15 g, 59 mmol) and bis(pinacolato)diboron (16.5 g, 64.9 mmol) were added to 300 ml of 1,4-dioxane, and the mixture was stirred and refluxed. Then, potassium acetate (8.7 g, 88.5 mmol) was added thereto, and the mixture was sufficiently stirred, and then bis(dibenzylideneacetone)palladium(0) (1 g, 1.8 mmol) and tricyclohexylphosphine (1 g, 3.5 mmol) were added. After the reaction for 4 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.6 g of sub1-6-2. (Yield: 65%, MS: [M+H]+=302)

sub1-6-2 (15 g, 49.8 mmol) and Trz40 (22.3 g, 52.3 mmol) were added to 300 ml of THF, and the mixture was stirred and refluxed. Then, potassium carbonate (20.6 g, 149.4 mmol) was dissolved in 62 ml of water and added thereto, and the mixture was sufficiently stirred, and then bis(tri-tert-butylphosphine)palladium(0) (0.3 g, 0.5 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 20.3 g of Compound 1-41. (Yield: 72%, MS: [M+H]+=566)

### Preparation Example 1-42

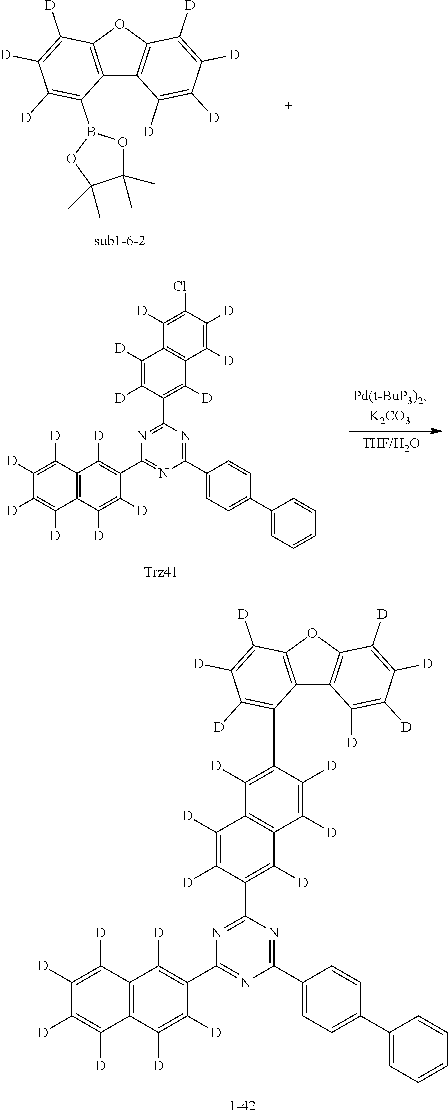 [SMILES: [2H]c1c([2H])c([2H])c2c(oc3c([2H])c([2H])c([2H])c(B4OC(C)(C)C(C)(C)O4)c32)c1[2H] ; [2H]c1c([2H])c([2H])c2c([2H])c(-c3nc(-c4ccc(-c5ccccc5)cc4)nc(-c4c([2H])c([2H])c5c([2H])c(Cl)c([2H])c([2H])c5c4[2H])n3)c([2H])c([2H])c2c1[2H] ; [2H]c1c([2H])c([2H])c2c(oc3c([2H])c([2H])c([2H])c(-c4c([2H])c([2H])c5c([2H])c(-c6nc(-c7ccc(-c8ccccc8)cc7)nc(-c7c([2H])c([2H])c8c([2H])c([2H])c([2H])c([2H])c8c7[2H])n6)c([2H])c([2H])c5c4[2H])c32)c1[2H]]

sub1-6-2 (15 g, 49.8 mmol) and Trz41 (27.9 g, 52.3 mmol) were added to 300 ml of THF, and the mixture was stirred and refluxed. Then, potassium carbonate (20.6 g, 149.4 mmol) was dissolved in 62 ml of water and added thereto, and the mixture was sufficiently stirred, and then bis(tri-tert-butylphosphine)palladium(0) (0.3 g, 0.5 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 24.7 g of Compound 1-42. (Yield: 74%, MS: [M+H]+=672)

### Preparation Example 1-43

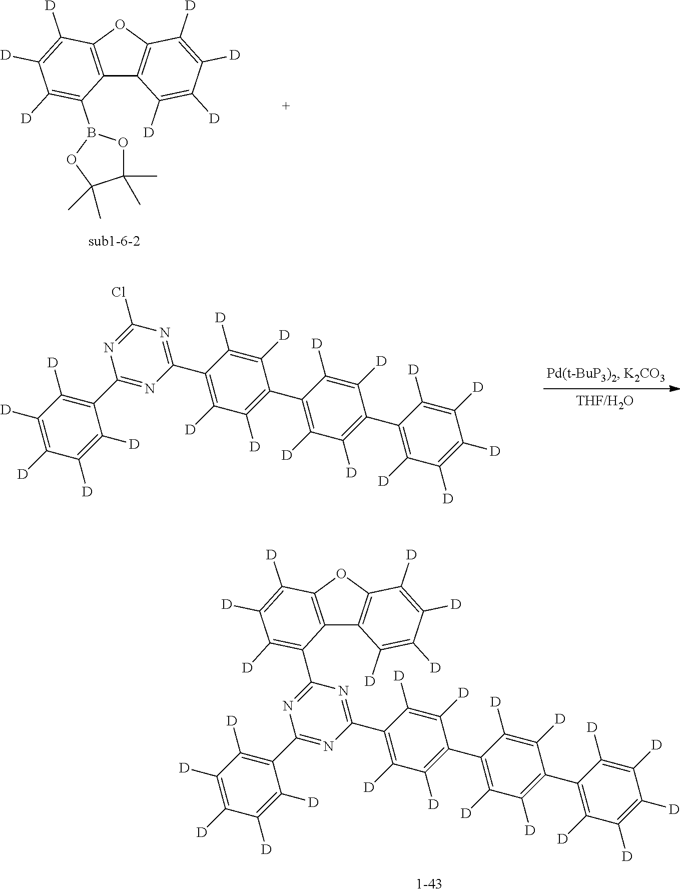 [SMILES: [2H]c1c([2H])c([2H])c2c(oc3c([2H])c([2H])c([2H])c(B4OC(C)(C)C(C)(C)O4)c32)c1[2H] ; [2H]c1c([2H])c([2H])c(-c2nc(Cl)nc(-c3c([2H])c([2H])c(-c4c([2H])c([2H])c(-c5c([2H])c([2H])c([2H])c([2H])c5[2H])c([2H])c4[2H])c([2H])c3[2H])n2)c([2H])c1[2H] ; [2H]c1c([2H])c([2H])c(-c2nc(-c3c([2H])c([2H])c(-c4c([2H])c([2H])c(-c5c([2H])c([2H])c([2H])c([2H])c5[2H])c([2H])c4[2H])c([2H])c3[2H])nc(-c3c([2H])c([2H])c([2H])c4oc5c([2H])c([2H])c([2H])c([2H])c5c34)n2)c([2H])c1[2H]]

sub1-6-2 (15 g, 49.8 mmol) and Trz42 (22.9 g, 52.3 mmol) were added to 300 ml of THF, and the mixture was stirred and refluxed. Then, potassium carbonate (20.6 g, 149.4 mmol) was dissolved in 62 ml of water and added thereto, and the mixture was sufficiently stirred, and then bis(tri-tert-butylphosphine)palladium(0) (0.3 g, 0.5 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 18.7 g of Compound 1-43. (Yield: 65%, MS: [M+H]+=577)

### Preparation Example 1-44

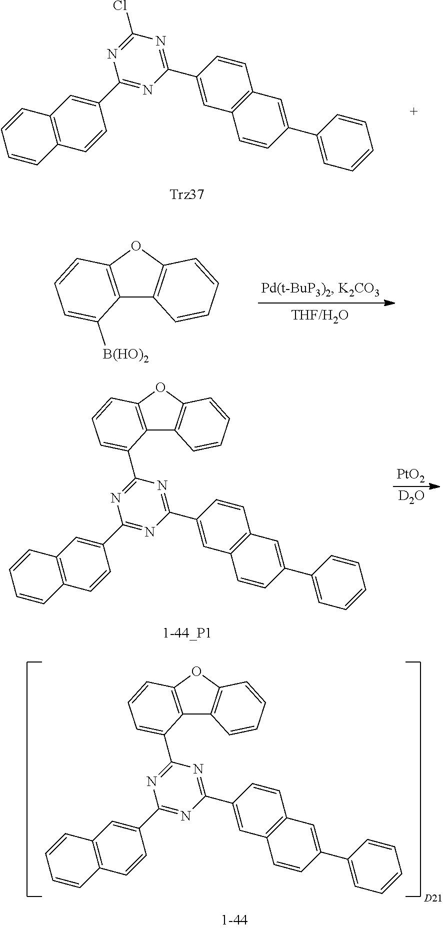

Trz37 (15 g, 33.8 mmol) and dibenzo[b,d]furan-1-ylboronic acid (7.5 g, 35.5 mmol) were added to 300 ml of THF, and the mixture was stirred and refluxed. Then, potassium carbonate (14 g, 101.4 mmol) was dissolved in 42 ml of water and added thereto, and the mixture was sufficiently stirred, and then bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.8 g of Compound 1-44_P1. (Yield: 66%, MS: [M+H]+=576)

Compound 1-44_P1 (10 g, 17.4 mmol), PtO₂ (1.2 g, 5.2 mmol) and D₂O (87 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 4.1 g of Compound 1-44. (Yield: 40%, MS: [M+H]⁺=598)

### Preparation Example 1-45

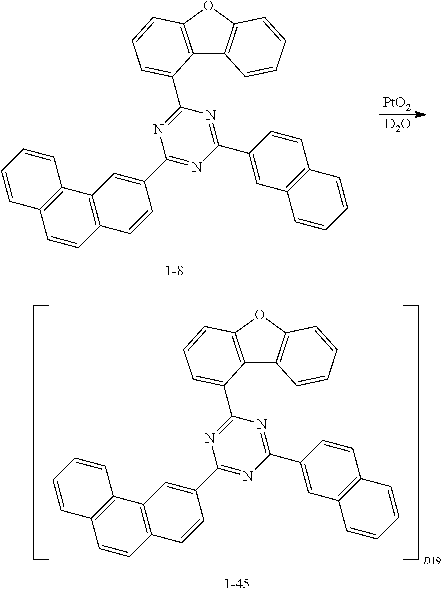

Compound 1-8 (10 g, 18.2 mmol), PtO₂ (1.2 g, 5.5 mmol) and D₂O (91 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 4.1 g of Compound 1-45. (Yield: 40%, MS: [M+H]+=570)

### Preparation Example 1-46

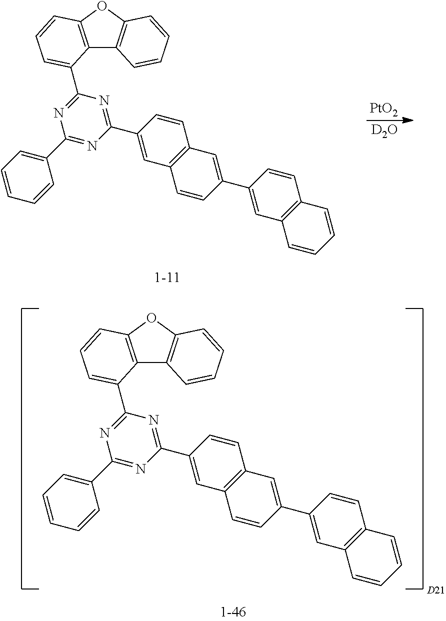

Compound 1-11 (10 g, 17.4 mmol), PtO₂ (1.2 g, 5.2 mmol) and D₂O (87 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 4.5 g of Compound 1-46. (Yield: 43%, MS: [M+H]+=598)

### Preparation Example 1-47

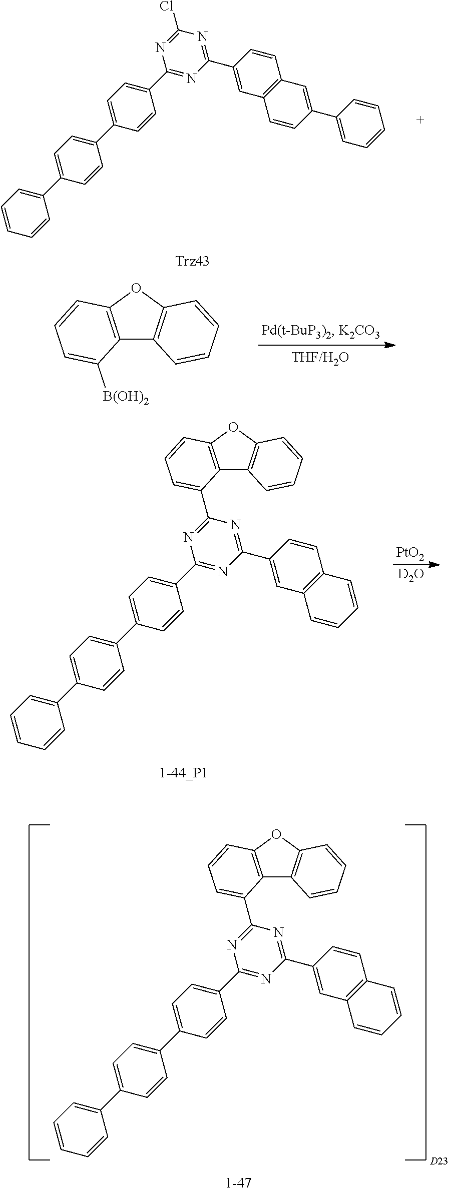

Trz43 (15 g, 31.9 mmol) and dibenzo[b,d]furan-1-ylboronic acid (7.1 g, 33.5 mmol) were added to 300 ml of THF, and the mixture was stirred and refluxed. Then, potassium carbonate (13.2 g, 95.8 mmol) was dissolved in 40 ml of water and added thereto, and the mixture was sufficiently stirred, and then bis(tri-tert-butylphosphine)palladium(0) (0.2 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 14.2 g of Compound 1-47_P1. (Yield: 74%, MS: [M+H]+=602)

Compound 1-47_P1 (10 g, 16.6 mmol), PtO₂ (1.1 g, 5 mmol) and D₂O (83 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 4.5 g of Compound 1-47. (Yield: 43%, MS: [M+H]+=626)

### Preparation Example 1-48

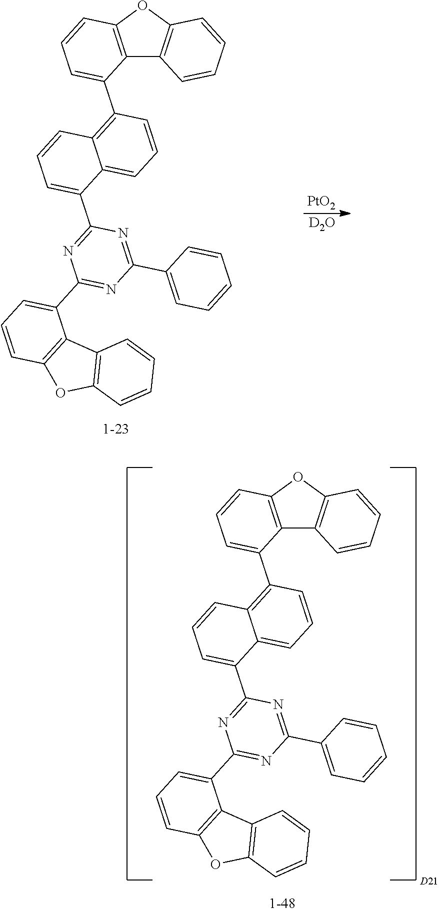

Compound 1-23 (10 g, 16.2 mmol), PtO₂ (1.1 g, 4.9 mmol) and D₂O (81 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 5 g of Compound 1-48. (Yield: 48%, MS: [M+H]+=638)

### Preparation Example 1-49

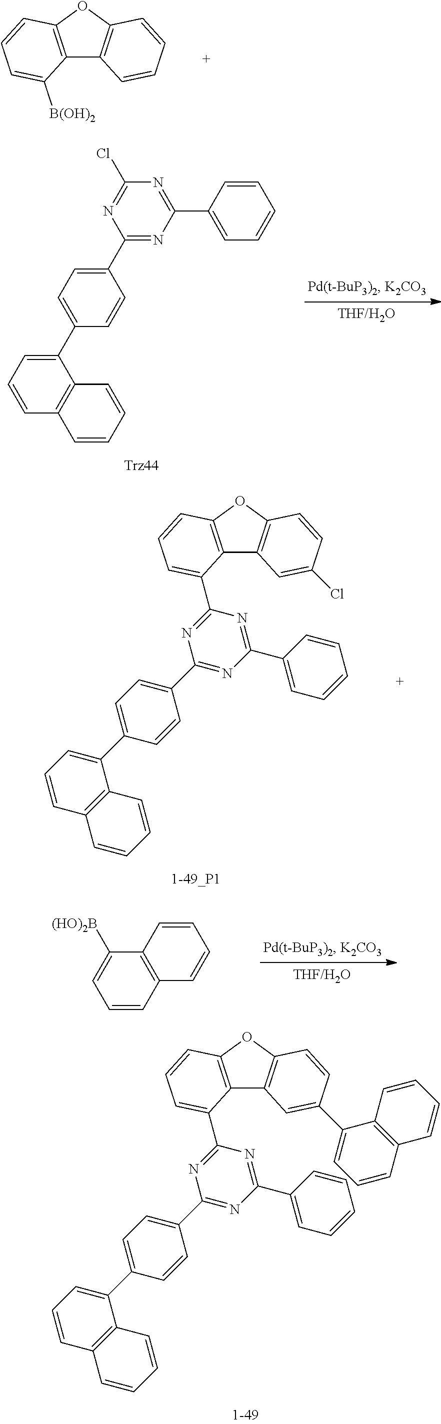 [SMILES: OB(O)c1cccc2oc3ccccc3c12 ; Clc1nc(-c2ccccc2)nc(-c2ccc(-c3cccc4ccccc34)cc2)n1 ; Clc1ccc2oc3cccc(-c4nc(-c5ccccc5)nc(-c5ccc(-c6cccc7ccccc67)cc5)n4)c3c2c1 ; OB(O)c1cccc2ccccc12 ; c1ccc(-c2nc(-c3ccc(-c4cccc5ccccc45)cc3)nc(-c3cccc4oc5ccc(-c6cccc7ccccc67)cc5c34)n2)cc1]

(8-Chlorodibenzo[b,d]furan-1-yl)boronic acid (15 g, 60.9 mmol) and Trz44 (25.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 23.5 g of Compound 1-49_P1. (Yield: 69%, MS: [M+H]+=560)

Compound 1-49_P1 (15 g, 26.8 mmol) and naphthalen-1-ylboronic acid (4.8 g, 28.1 mmol) were added to 300 ml of THF, and the mixture was stirred and refluxed. Then, potassium carbonate (11.1 g, 80.3 mmol) was dissolved in 33 ml of water and added thereto, and the mixture was sufficiently stirred, and then bis(tri-tert-butylphosphine)palladium(0) (0.1 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.7 g of Compound 1-49. (Yield: 73%, MS: [M+H]+=652)

### Preparation Example 1-50

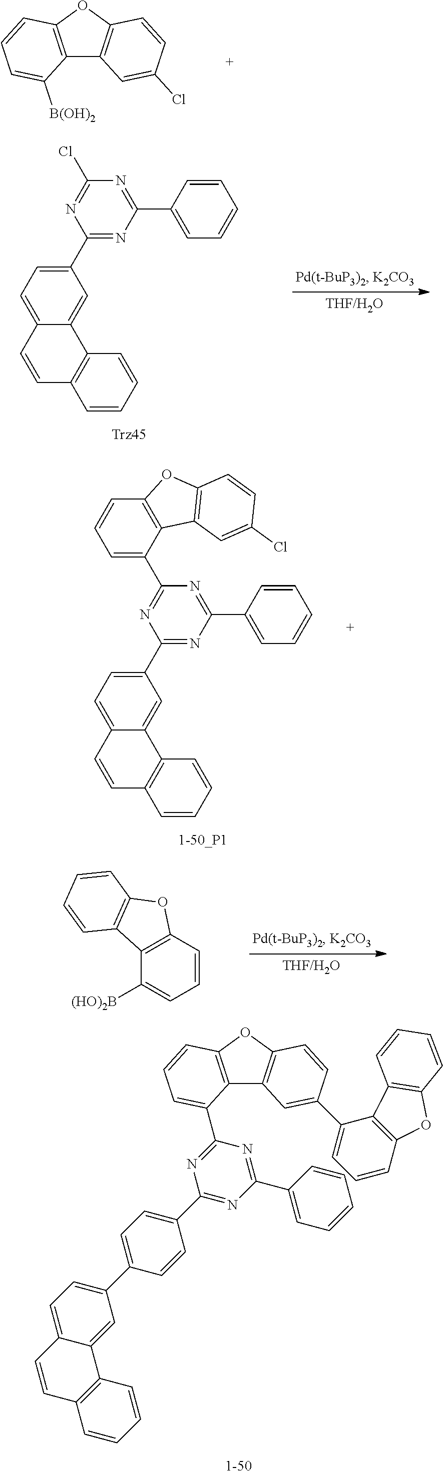

(8-Chlorodibenzo[b,d]furan-1-yl)boronic acid (15 g, 60.9 mmol) and Trz45 (23.5 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 24 g of Compound 1-50_P1. (Yield: 74%, MS: [M+H]+=534)

Compound 1-50_P1 (15 g, 28.1 mmol) and dibenzo[b,d]furan-1-ylboronic acid (6.3 g, 29.5 mmol) were added to 300 ml of THF, and the mixture was stirred and refluxed. Then, potassium carbonate (11.7 g, 84.4 mmol) was dissolved in 35 ml of water and added thereto, and the mixture was sufficiently stirred, and then bis(tri-tert-butylphosphine)palladium(0) (0.1 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.4 g of Compound 1-50. (Yield: 66%, MS: [M+H]+=666)

### Preparation Example 1-51

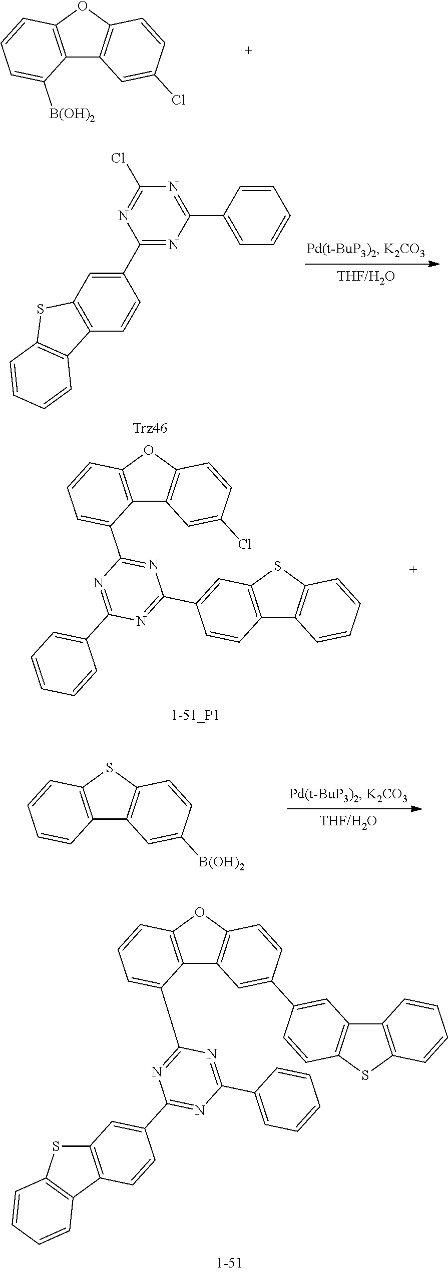 [SMILES: OB(O)c1cccc2oc3ccc(Cl)cc3c12 ; Clc1nc(-c2ccccc2)nc(-c2ccc3c(c2)sc2ccccc23)n1 ; Clc1ccc2oc3cccc(-c4nc(-c5ccccc5)nc(-c5ccc6c(c5)sc5ccccc56)n4)c3c2c1 ; OB(O)c1ccc2sc3ccccc3c2c1 ; c1ccc(-c2nc(-c3ccc4c(c3)sc3ccccc34)nc(-c3cccc4oc5ccc(-c6ccc7sc8ccccc8c7c6)cc5c34)n2)cc1]

(8-Chlorodibenzo[b,d]furan-1-yl)boronic acid (15 g, 60.9 mmol) and Trz46 (23.9 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 23.6 g of Compound 1-51_P1. (Yield: 72%, MS: [M+H]+=540)

Compound 1-51_P1 (15 g, 27.8 mmol) and dibenzo[b,d]thiophen-2-ylboronic acid (6.7 g, 29.2 mmol) were added to 300 ml of THF, and the mixture was stirred and refluxed. Then, potassium carbonate (11.5 g, 83.3 mmol) was dissolved in 35 ml of water and added thereto, and the mixture was sufficiently stirred, and then bis(tri-tert-butylphosphine)palladium(0) (0.1 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.4 g of Compound 1-51. (Yield: 65%, MS: [M+H]+=688)

### Preparation Example 1-52

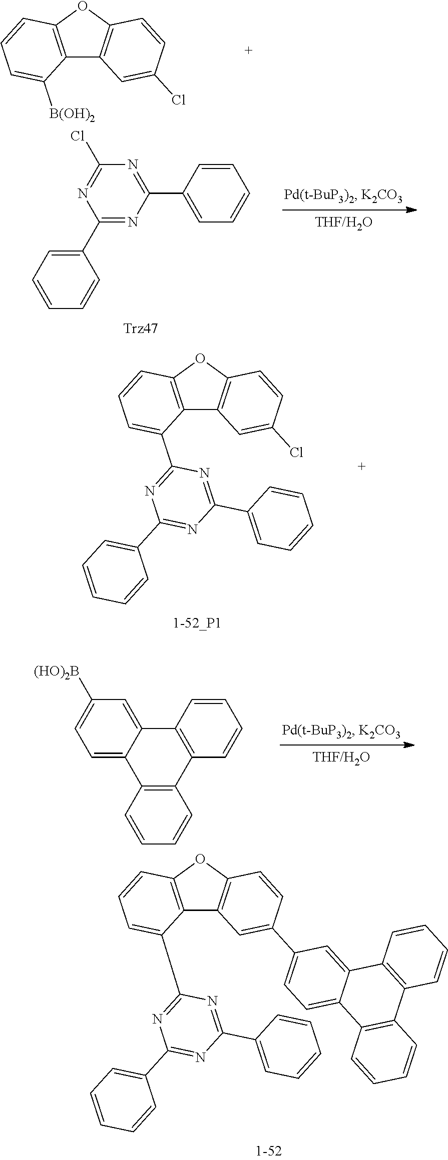

(8-Chlorodibenzo[b,d]furan-1-yl)boronic acid (15 g, 60.9 mmol) and Trz47 (17.1 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 17.9 g of Compound 1-52_P1. (Yield: 68%, MS: [M+H]+=434)

Compound 1-52_P1 (15 g, 34.6 mmol) and triphenylen-2-ylboronic acid (9.9 g, 36.3 mmol) were added to 300 ml of THF, and the mixture was stirred and refluxed. Then, potassium carbonate (14.3 g, 103.7 mmol) was dissolved in 43 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 14.3 g of Compound 1-52. (Yield: 66%, MS: [M+H]+=626)

### Preparation Example 1-53

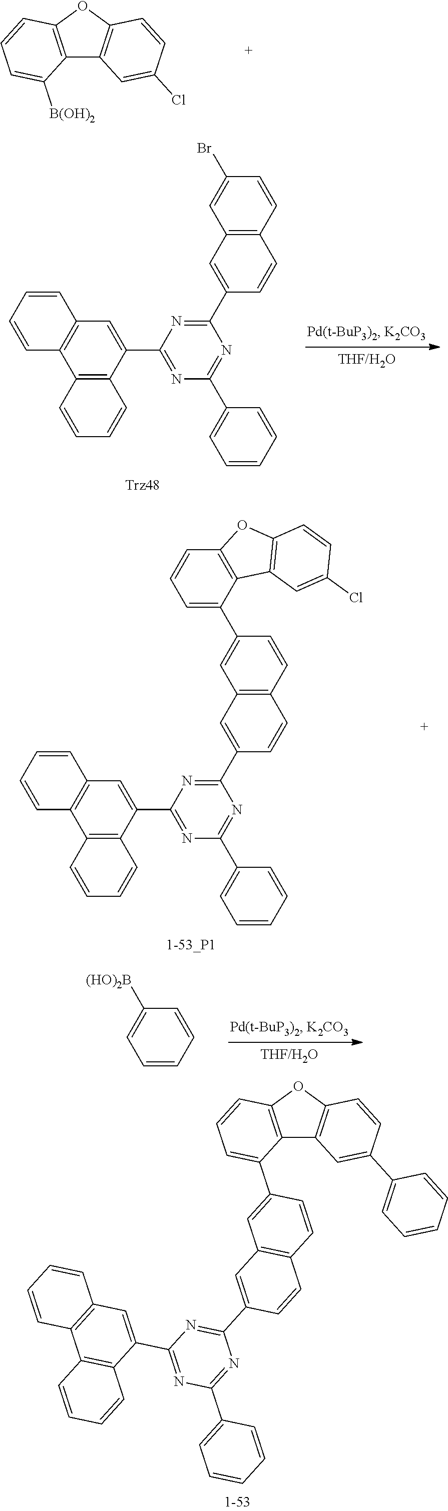 [SMILES: OB(O)c1cccc2oc3ccc(Cl)cc3c12 ; Brc1ccc2ccc(-c3nc(-c4ccccc4)nc(-c4cc5ccccc5c5ccccc45)n3)cc2c1 ; Clc1ccc2oc3cccc(-c4ccc5ccc(-c6nc(-c7ccccc7)nc(-c7cc8ccccc8c8ccccc78)n6)cc5c4)c3c2c1 ; OB(O)c1ccccc1 ; c1ccc(-c2ccc3oc4cccc(-c5ccc6ccc(-c7nc(-c8ccccc8)nc(-c8cc9ccccc9c9ccccc89)n7)cc6c5)c4c3c2)cc1]

(8-Chlorodibenzo[b,d]furan-1-yl)boronic acid (15 g, 60.9 mmol) and Trz48 (34.4 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 30.1 g of Compound 1-53_P1. (Yield: 75%, MS: [M+H]+=660)

Compound 1-53_P1 (15 g, 22.7 mmol) and phenylboronic acid (2.9 g, 23.9 mmol) were added to 300 ml of THF, and the mixture was stirred and refluxed. Then, potassium carbonate (9.4 g, 68.2 mmol) was dissolved in 28 ml of water and added thereto, and the mixture was sufficiently stirred, and then bis(tri-tert-butylphosphine)palladium(0) (0.1 g, 0.2 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 10.7 g of Compound 1-53. (Yield: 67%, MS: [M+H]+=702)

### Preparation Example 1-54

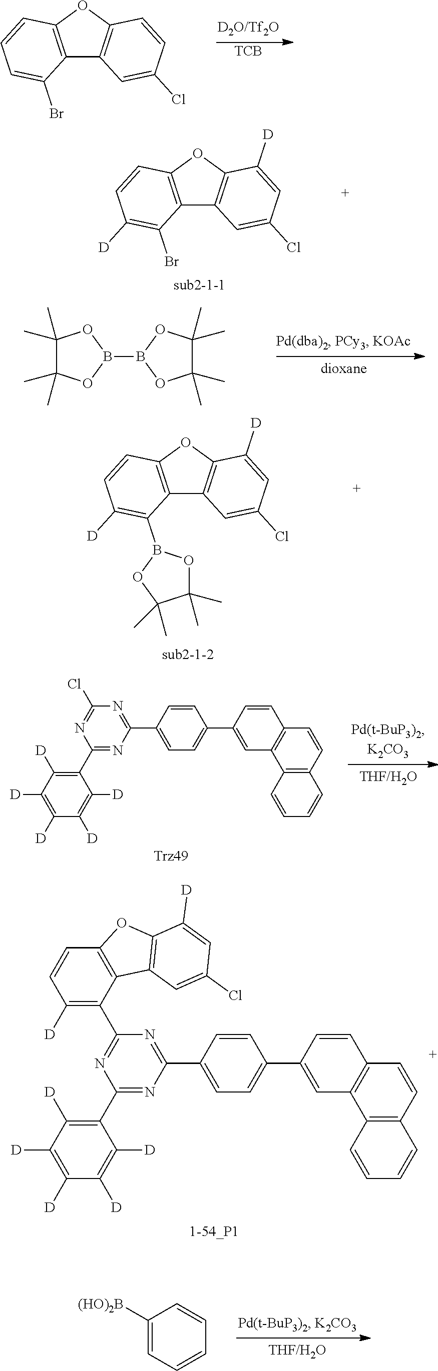

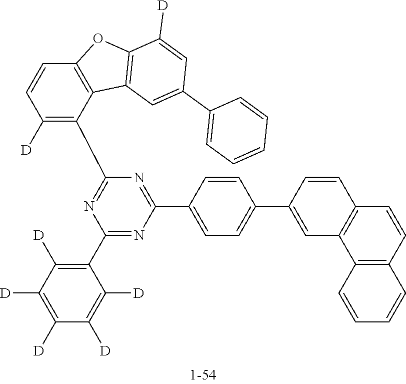 [SMILES: [2H]c1ccc2oc3c([2H])cc(-c4ccccc4)cc3c2c1-c1nc(-c2ccc(-c3ccc4ccc5ccccc5c4c3)cc2)nc(-c2c([2H])c([2H])c([2H])c([2H])c2[2H])n1]

Trifluoromethanesulfonic anhydride (30.1 g, 106.6 mmol) and deuterium oxide (10.7 g, 532.8 mmol) were added at 0° C. and stirred for 5 hours to prepare a solution. 1-Bromo-8-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-8-chlorodibenzo[b,d]furan and 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.5 g of sub2-1-1. (Yield: 43%, MS: [M+H]+=283)

Sub2-1-1 (15 g, 52.9 mmol) and bis(pinacolato)diboron (14.8 g, 58.2 mmol) were added to 300 ml of 1,4-dioxane, and the mixture was stirred and refluxed. Then, potassium acetate (7.8 g, 79.4 mmol) was added thereto, and the mixture was sufficiently stirred, and then bis(dibenzylideneacetone)palladium(0) (0.9 g, 1.6 mmol) and tricyclohexylphosphine (0.9 g, 3.2 mmol) were added. After the reaction for 5 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.5 g of sub2-1-2. (Yield: 66%, MS: [M+H]+=331)

Sub2-1-2 (15 g, 45.4 mmol) and Trz49 (21.4 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred, and then bis(tri-tert-butylphosphine)palladium(0) (0.2 g, 0.5 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 18.2 g of Compound 1-54_P1. (Yield: 65%, MS: [M+H]+=617)

Compound 1-54_P1 (15 g, 24.3 mmol) and phenylboronic acid (3.1 g, 25.5 mmol) were added to 300 ml of THF, and the mixture was stirred and refluxed. Then, potassium carbonate (10.1 g, 72.9 mmol) was dissolved in 30 ml of water and added thereto, and the mixture was sufficiently stirred, and then bis(tri-tert-butylphosphine)palladium(0) (0.1 g, 0.2 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11 g of Compound 1-54. (Yield: 69%, MS: [M+H]+=659)

### Preparation Example 1-55

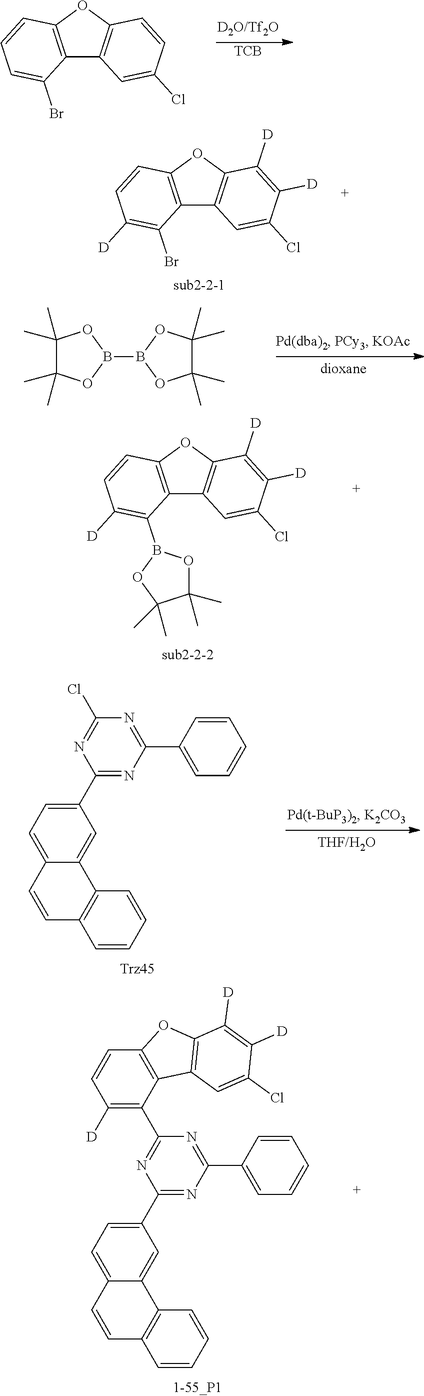

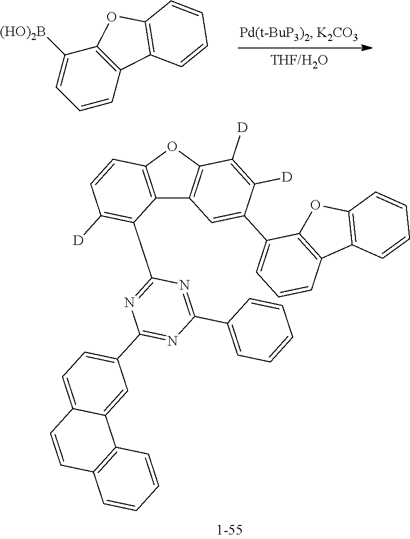 [SMILES: OB(O)c1cccc2c1oc1ccccc12 ; [2H]c1ccc2oc3c([2H])c([2H])c(-c4cccc5c4oc4ccccc45)cc3c2c1-c1nc(-c2ccccc2)nc(-c2ccc3ccc4ccccc4c3c2)n1]

Trifluoromethanesulfonic anhydride (45.1 g, 159.8 mmol) and deuterium oxide (16 g, 799.2 mmol) were added at 0° C. and stirred for 5 hours to prepare a solution. 1-Bromo-8-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-8-chlorodibenzo[b,d]furan and 1,2,4-trichlorobenzene, and the mixture was stirred while heating to 140° C. and then keeping that temperature. After the reaction for 7 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.6 g of sub2-2-1. (Yield: 37%, MS: [M+H]+=284)

Sub2-2-1 (15 g, 52.7 mmol) and bis(pinacolato)diboron (14.7 g, 58 mmol) were added to 300 ml of 1,4-dioxane, and the mixture was stirred and refluxed. Then, potassium acetate (7.8 g, 79.1 mmol) was added thereto, and the mixture was sufficiently stirred, and then bis(dibenzylideneacetone)palladium(0) (0.9 g, 1.6 mmol) and tricyclohexylphosphine (0.9 g, 3.2 mmol) were added. After the reaction for 6 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 10.1 g of sub2-2-2. (Yield: 58%, MS: [M+H]+=332)

Sub2-2-2 (15 g, 45.2 mmol) and Trz45 (17.5 g, 47.5 mmol) were added to 300 ml of THF, and the mixture was stirred and refluxed. Then, potassium carbonate (18.8 g, 135.7 mmol) was dissolved in 56 ml of water and added thereto, and the mixture was sufficiently stirred, and then bis(tri-tert-butylphosphine)palladium(0) (0.2 g, 0.5 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 17 g of Compound 1-55_P1. (Yield: 70%, MS: [M+H]+=537)

Compound 1-55_P1 (15 g, 24.3 mmol) and phenylboronic acid (3.1 g, 25.5 mmol) were added to 300 ml of THF, and the mixture was stirred and refluxed. Then, potassium carbonate (10.1 g, 72.9 mmol) was dissolved in 30 ml of water and added thereto, and the mixture was sufficiently stirred, and then bis(tri-tert-butylphosphine)palladium(0) (0.1 g, 0.2 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11 g of Compound 1-55. (Yield: 69%, MS: [M+H]+=659)

### Preparation Example 1-56

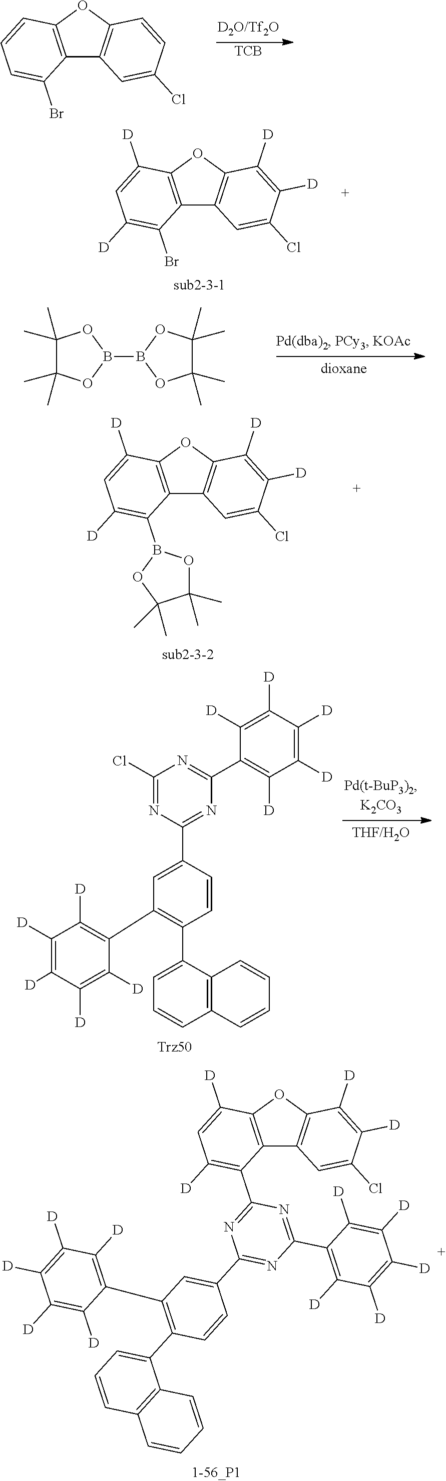

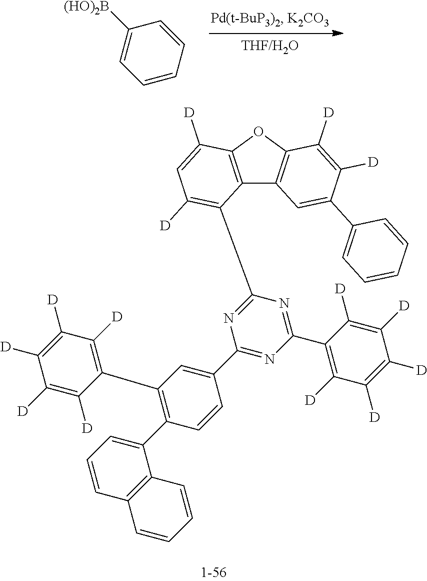

Trifluoromethanesulfonic anhydride (60.1 g, 213.1 mmol) and deuterium oxide (21.4 g, 1065.6 mmol) were added at 0° C. and stirred for 5 hours to prepare a solution. 1-Bromo-8-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-8-chlorodibenzo[b,d]furan and 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.4 g of sub2-3-1. (Yield: 42%, MS: [M+H]+=285)

Sub2-3-1 (15 g, 52.5 mmol) and bis(pinacolato)diboron (14.7 g, 57.8 mmol) were added to 300 ml of 1,4-dioxane, and the mixture was stirred and refluxed. Then, potassium acetate (7.7 g, 78.8 mmol) was added thereto, and the mixture was sufficiently stirred, and then bis(dibenzylideneacetone)palladium(0) (0.9 g, 1.6 mmol) and tricyclohexylphosphine (0.9 g, 3.2 mmol) were added. After the reaction for 6 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12 g of sub2-3-2. (Yield: 69%, MS: [M+H]+=333)

Sub2-3-2 (15 g, 45.1 mmol) and Trz50 (22.7 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred, and then bis(tri-tert-butylphosphine)palladium(0) (0.2 g, 0.5 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 20.2 g of Compound 1-56_P1. (Yield: 69%, MS: [M+H]+=650)

Compound 1-56_P1 (15 g, 23.1 mmol) and phenylboronic acid (2.9 g, 24.2 mmol) were added to 300 ml of THF, and the mixture was stirred and refluxed. Then, potassium carbonate (9.6 g, 69.2 mmol) was dissolved in 29 ml of water and added thereto, and the mixture was sufficiently stirred, and then bis(tri-tert-butylphosphine)palladium(0) (0.1 g, 0.2 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 10.5 g of Compound 1-56. (Yield: 66%, MS: [M+H]+=692)

### Preparation Example 1-57

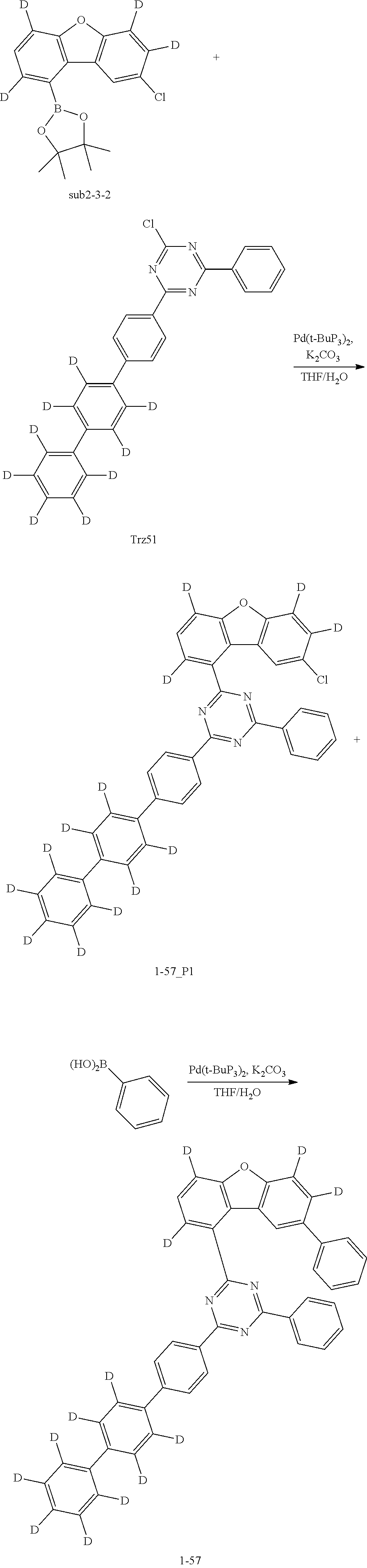 [SMILES: [2H]c1cc([2H])c2oc3c([2H])c([2H])c(Cl)cc3c2c1B1OC(C)(C)C(C)(C)O1 ; [2H]c1c([2H])c([2H])c(-c2c([2H])c([2H])c(-c3ccc(-c4nc(Cl)nc(-c5ccccc5)n4)cc3)c([2H])c2[2H])c([2H])c1[2H] ; [2H]c1cc([2H])c2oc3c([2H])c([2H])c(Cl)cc3c2c1-c1nc(-c2ccccc2)nc(-c2ccc(-c3c([2H])c([2H])c(-c4c([2H])c([2H])c([2H])c([2H])c4[2H])c([2H])c3[2H])cc2)n1 ; OB(O)c1ccccc1 ; [2H]c1cc([2H])c2oc3c([2H])c([2H])c(-c4ccccc4)cc3c2c1-c1nc(-c2ccccc2)nc(-c2ccc(-c3c([2H])c([2H])c(-c4c([2H])c([2H])c([2H])c([2H])c4[2H])c([2H])c3[2H])cc2)n1]

Sub2-3-2 (15 g, 45.1 mmol) and Trz51 (20.3 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred, and then bis(tri-tert-butylphosphine)palladium(0) (0.2 g, 0.5 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 20.2 g of Compound 1-57_P1. (Yield: 75%, MS: [M+H]+=599)

Compound 1-57_P1 (15 g, 25 mmol) and phenylboronic acid (3.2 g, 26.3 mmol) were added to 300 ml of THF, and the mixture was stirred and refluxed. Then, potassium carbonate (10.4 g, 75.1 mmol) was dissolved in 31 ml of water and added thereto, and the mixture was sufficiently stirred, and then bis(tri-tert-butylphosphine)palladium(0) (0.1 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.9 g of Compound 1-57. (Yield: 74%, MS: [M+H]+=641)

### Preparation Example 1-58

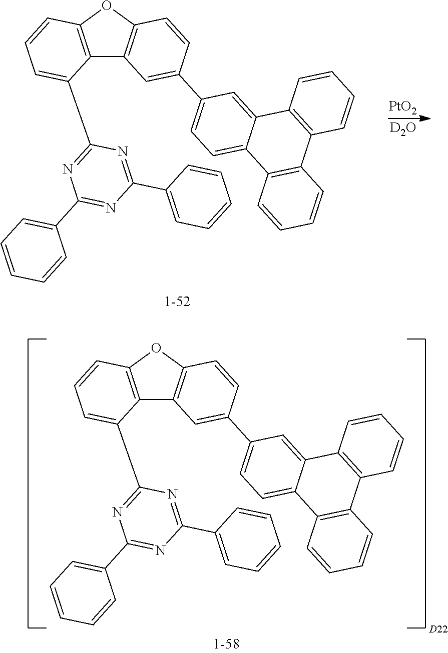

Compound 1-52 (10 g, 16 mmol), PtO₂ (1.1 g, 4.8 mmol) and D₂O (80 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 3.9 g of Compound 1-58. (Yield: 38%, MS: [M+H]+=649)

### Preparation Example 1-59

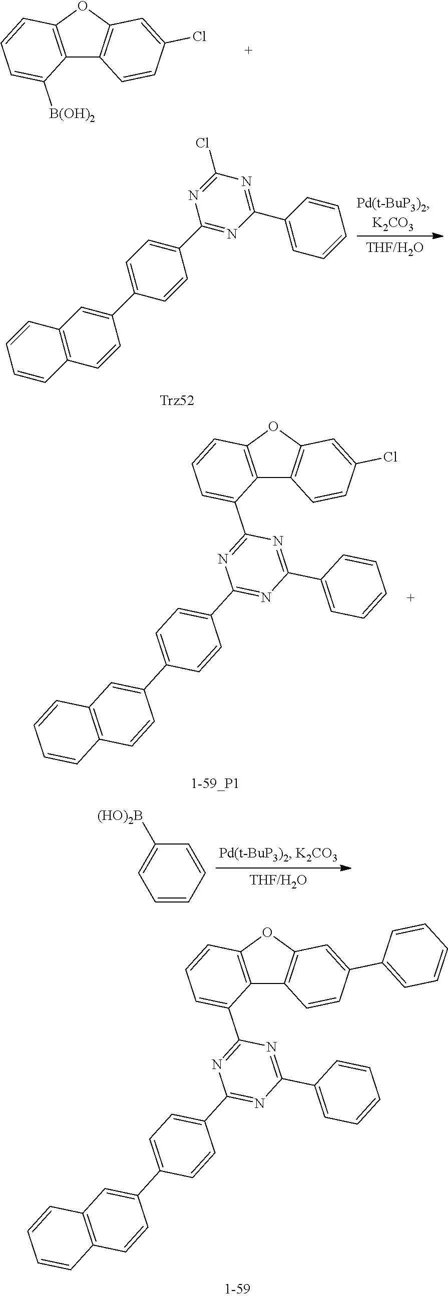 [SMILES: OB(O)c1cccc2oc3cc(Cl)ccc3c12 ; Clc1nc(-c2ccccc2)nc(-c2ccc(-c3ccc4ccccc4c3)cc2)n1 ; Clc1ccc2c(c1)oc1cccc(-c3nc(-c4ccccc4)nc(-c4ccc(-c5ccc6ccccc6c5)cc4)n3)c12 ; OB(O)c1ccccc1 ; c1ccc(-c2ccc3c(c2)oc2cccc(-c4nc(-c5ccccc5)nc(-c5ccc(-c6ccc7ccccc7c6)cc5)n4)c23)cc1]

(7-Chlorodibenzo[b,d]furan-1-yl)boronic acid (15 g, 60.9 mmol) and Trz52 (25.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 25.2 g of Compound 1-59_P1. (Yield: 74%, MS: [M+H]+=560)

Compound 1-59_P1 (15 g, 26.8 mmol) and phenylboronic acid (3.4 g, 28.1 mmol) were added to 300 ml of THF, and the mixture was stirred and refluxed. Then, potassium carbonate (11.1 g, 80.3 mmol) was dissolved in 33 ml of water and added thereto, and the mixture was sufficiently stirred, and then bis(tri-tert-butylphosphine)palladium(0) (0.1 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.6 g of Compound 1-59. (Yield: 72%, MS: [M+H]+=602)

### Preparation Example 1-60

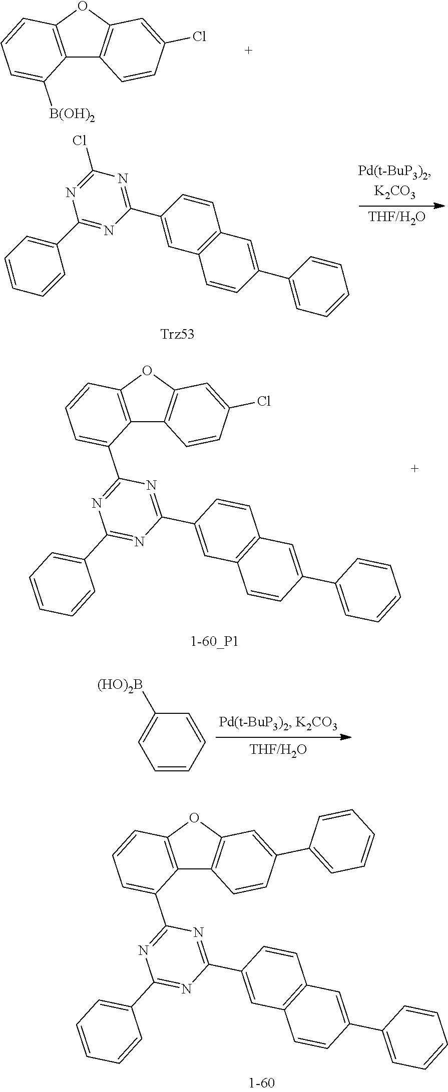

(7-Chlorodibenzo[b,d]furan-1-yl)boronic acid (15 g, 60.9 mmol) and Trz53 (25.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 25.2 g of Compound 1-60_P1. (Yield: 74%, MS: [M+H]+=560)

Compound 1-60_P1 (15 g, 26.8 mmol) and phenylboronic acid (3.4 g, 28.1 mmol) were added to 300 ml of THF, and the mixture was stirred and refluxed. Then, potassium carbonate (11.1 g, 80.3 mmol) was dissolved in 33 ml of water and added thereto, and the mixture was sufficiently stirred, and then bis(tri-tert-butylphosphine)palladium(0) (0.1 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.1 g of Compound 1-60. (Yield: 75%, MS: [M+H]+=602)

### Preparation Example 1-61

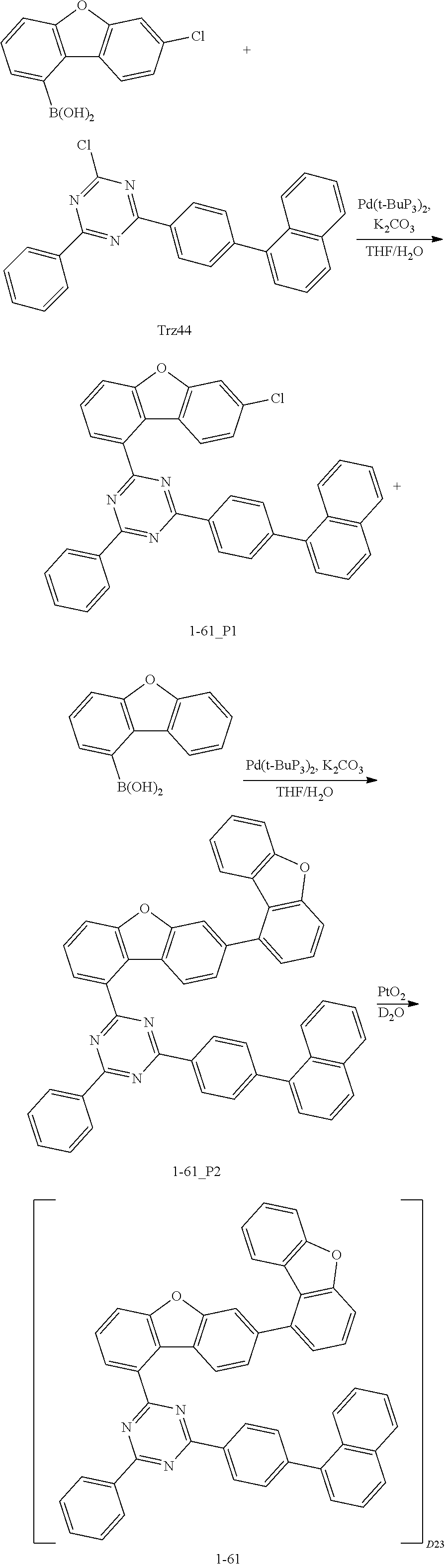

(7-Chlorodibenzo[b,d]furan-1-yl)boronic acid (15 g, 60.9 mmol) and Trz44 (25.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 25.2 g of Compound 1-61_P1. (Yield: 74%, MS: [M+H]+=560)

Compound 1-61_P1 (15 g, 26.8 mmol) and dibenzo[b,d]furan-1-ylboronic acid (6 g, 28.1 mmol) were added to 300 ml of THF, and the mixture was stirred and refluxed. Then, potassium carbonate (11.1 g, 80.3 mmol) was dissolved in 33 ml of water and added thereto, and the mixture was sufficiently stirred, and then bis(tri-tert-butylphosphine)palladium(0) (0.1 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.9 g of Compound 1-61_P2. (Yield: 75%, MS: [M+H]+=692)

Compound 1-61_P2 (10 g, 14.5 mmol), PtO₂ (1 g, 4.3 mmol) and D₂O (72 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 3.8 g of Compound 1-61. (Yield: 37%, MS: [M+H]+=716)

### Preparation Example 1-62

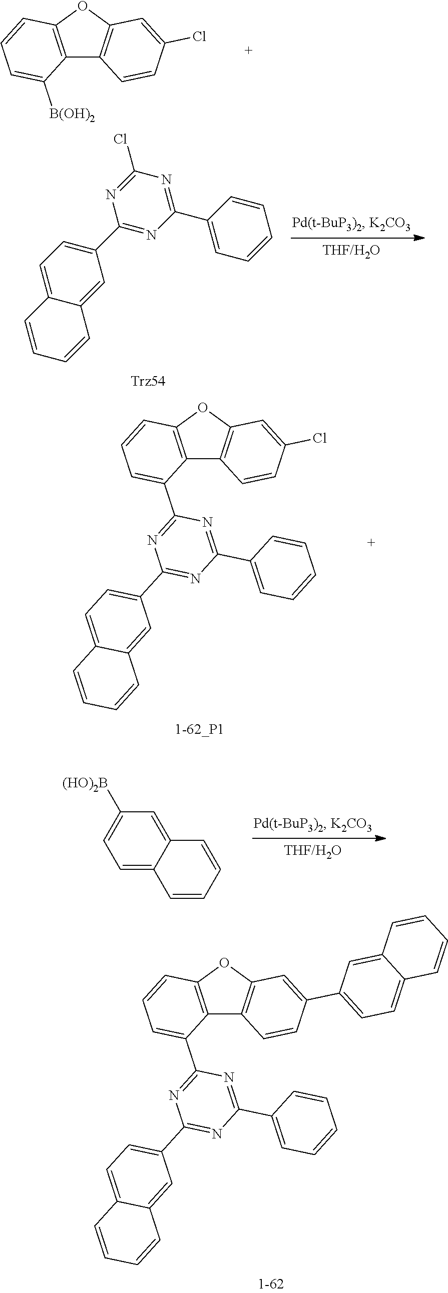 [SMILES: OB(O)c1cccc2oc3cc(Cl)ccc3c12 ; Clc1nc(-c2ccccc2)nc(-c2ccc3ccccc3c2)n1 ; Clc1ccc2c(c1)oc1cccc(-c3nc(-c4ccccc4)nc(-c4ccc5ccccc5c4)n3)c12 ; OB(O)c1ccc2ccccc2c1 ; c1ccc(-c2nc(-c3ccc4ccccc4c3)nc(-c3cccc4oc5cc(-c6ccc7ccccc7c6)ccc5c34)n2)cc1]

(7-Chlorodibenzo[b,d]furan-1-yl)boronic acid (15 g, 60.9 mmol) and Trz54 (20.3 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 21.8 g of Compound 1-62_P1. (Yield: 74%, MS: [M+H]+=484)

Compound 1-62_P1 (15 g, 31 mmol) and naphthalen-2-ylboronic acid (5.6 g, 32.5 mmol) were added to 300 ml of THF, and the mixture was stirred and refluxed. Then, potassium carbonate (12.9 g, 93 mmol) was dissolved in 39 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.3 g of Compound 1-62. (Yield: 69%, MS: [M+H]+=576)

### Preparation Example 1-63

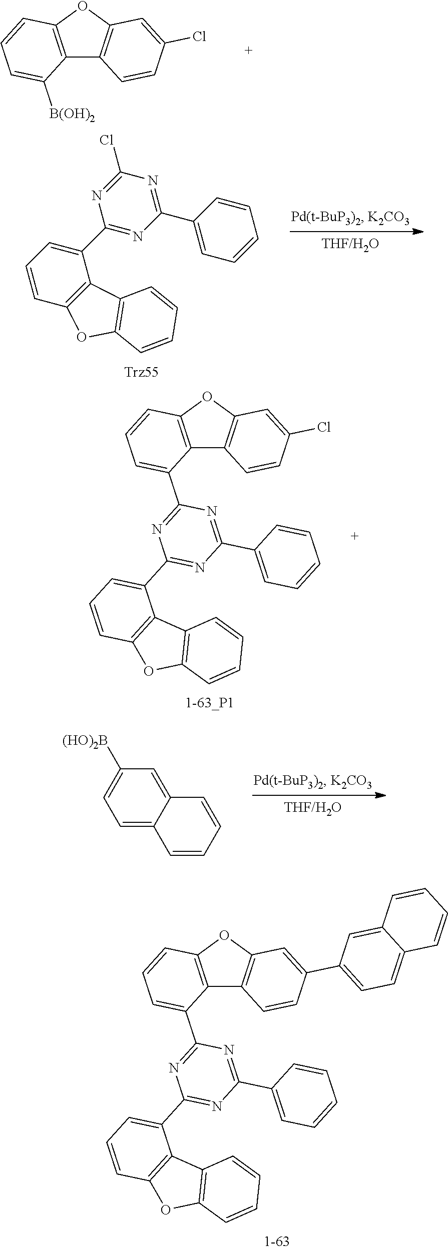 [SMILES: OB(O)c1cccc2oc3cc(Cl)ccc3c12 ; Clc1nc(-c2ccccc2)nc(-c2cccc3oc4ccccc4c23)n1 ; Clc1ccc2c(c1)oc1cccc(-c3nc(-c4ccccc4)nc(-c4cccc5oc6ccccc6c45)n3)c12 ; OB(O)c1ccc2ccccc2c1 ; c1ccc(-c2nc(-c3cccc4oc5ccccc5c34)nc(-c3cccc4oc5cc(-c6ccc7ccccc7c6)ccc5c34)n2)cc1]

(7-Chlorodibenzo[b,d]furan-1-yl)boronic acid (15 g, 60.9 mmol) and Trz55 (22.9 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 21 g of Compound 1-63_P1. (Yield: 66%, MS: [M+H]+=524)

Compound 1-63_P1 (15 g, 28.6 mmol) and naphthalen-2-ylboronic acid (5.2 g, 30.1 mmol) were added to 300 ml of THF, and the mixture was stirred and refluxed. Then, potassium carbonate (11.9 g, 85.9 mmol) was dissolved in 36 ml of water and added thereto, and the mixture was sufficiently stirred, and then bis(tri-tert-butylphosphine)palladium(0) (0.1 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.4 g of Compound 1-63. (Yield: 65%, MS: [M+H]+=616)

### Preparation Example 1-64

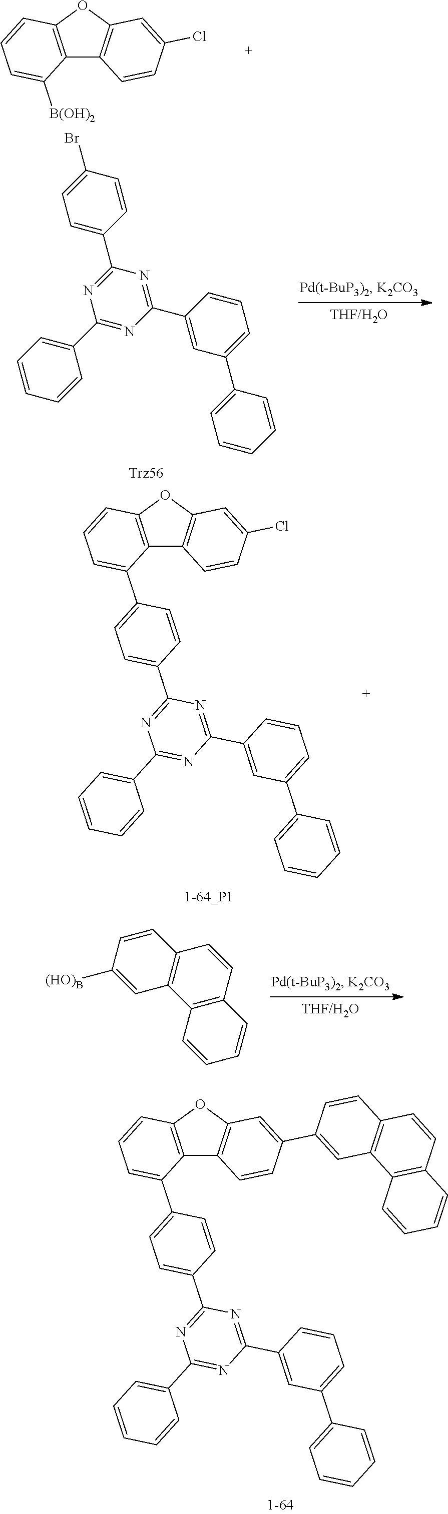

(7-Chlorodibenzo[b,d]furan-1-yl)boronic acid (15 g, 60.9 mmol) and Trz56 (29.7 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 23.9 g of Compound 1-64_P1. (Yield: 67%, MS: [M+H]+=586)

Compound 1-64_P1 (15 g, 25.6 mmol) and phenanthren-3-ylboronic acid (6 g, 26.9 mmol) were added to 300 ml of THF, and the mixture was stirred and refluxed. Then, potassium carbonate (10.6 g, 76.8 mmol) was dissolved in 32 ml of water and added thereto, and the mixture was sufficiently stirred, and then bis(tri-tert-butylphosphine)palladium(0) (0.1 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.3 g of Compound 1-64. (Yield: 66%, MS: [M+H]+=728)

### Preparation Example 1-65

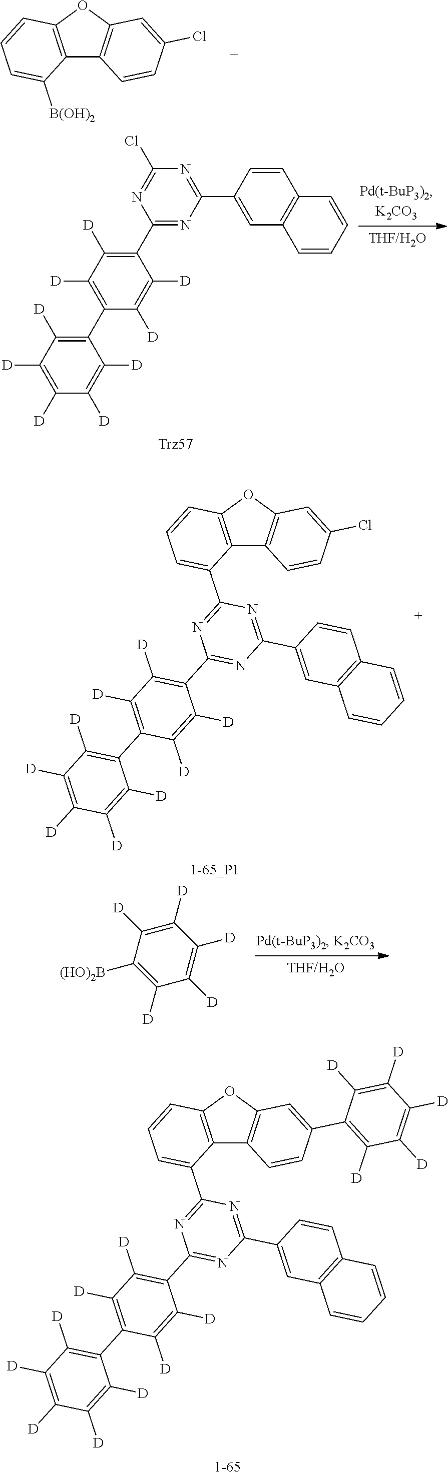 [SMILES: OB(O)c1cccc2oc3cc(Cl)ccc3c12 ; [2H]c1c([2H])c([2H])c(-c2c([2H])c([2H])c(-c3nc(Cl)nc(-c4ccc5ccccc5c4)n3)c([2H])c2[2H])c([2H])c1[2H] ; [2H]c1c([2H])c([2H])c(-c2c([2H])c([2H])c(-c3nc(-c4ccc5ccccc5c4)nc(-c4cccc5oc6cc(Cl)ccc6c45)n3)c([2H])c2[2H])c([2H])c1[2H] ; [2H]c1c([2H])c([2H])c(B(O)O)c([2H])c1[2H] ; [2H]c1c([2H])c([2H])c(-c2ccc3c(c2)oc2cccc(-c4nc(-c5ccc6ccccc6c5)nc(-c5c([2H])c([2H])c(-c6c([2H])c([2H])c([2H])c([2H])c6[2H])c([2H])c5[2H])n4)c23)c([2H])c1[2H]]

(7-Chlorodibenzo[b,d]furan-1-yl)boronic acid (15 g, 60.9 mmol) and Trz57 (25.8 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 24.6 g of Compound 1-65_P1. (Yield: 71%, MS: [M+H]+=569)

Compound 1-65_P1 (15 g, 26.4 mmol) and (phenyl-d5)boronic acid (3.5 g, 27.7 mmol) were added to 300 ml of THF, and the mixture was stirred and refluxed. Then, potassium carbonate (10.9 g, 79.1 mmol) was dissolved in 33 ml of water and added thereto, and the mixture was sufficiently stirred, and then bis(tri-tert-butylphosphine)palladium(0) (0.1 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.7 g of Compound 1-65. (Yield: 72%, MS: [M+H]+=616)

### Preparation Example 1-66

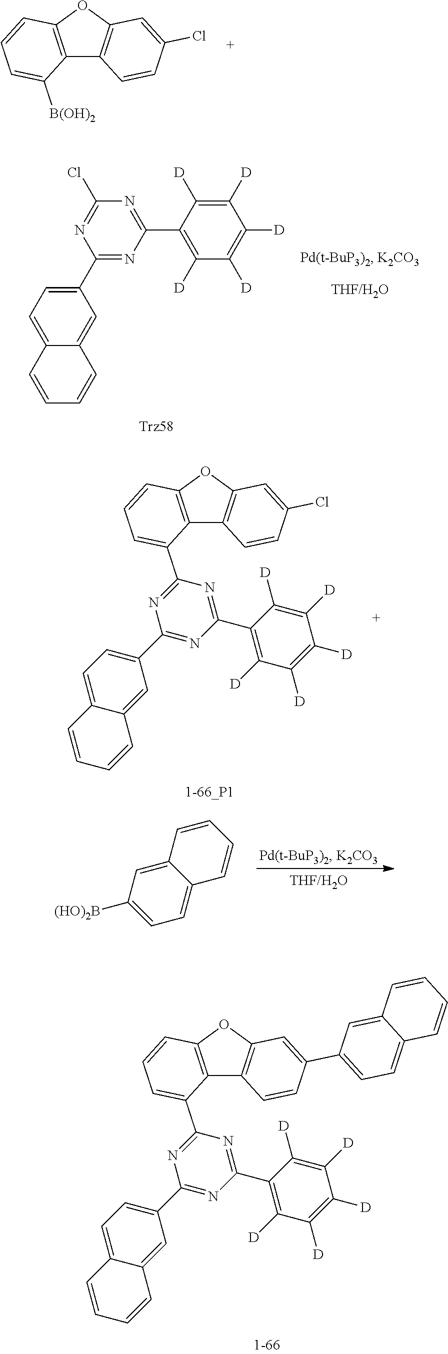 [SMILES: OB(O)c1cccc2oc3cc(Cl)ccc3c12 ; [2H]c1c([2H])c([2H])c(-c2nc(Cl)nc(-c3ccc4ccccc4c3)n2)c([2H])c1[2H] ; [2H]c1c([2H])c([2H])c(-c2nc(-c3ccc4ccccc4c3)nc(-c3cccc4oc5cc(Cl)ccc5c34)n2)c([2H])c1[2H] ; OB(O)c1ccc2ccccc2c1 ; [2H]c1c([2H])c([2H])c(-c2nc(-c3ccc4ccccc4c3)nc(-c3cccc4oc5cc(-c6ccc7ccccc7c6)ccc5c34)n2)c([2H])c1[2H]]

(7-Chlorodibenzo[b,d]furan-1-yl)boronic acid (15 g, 60.9 mmol) and Trz58 (20.6 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 20.2 g of Compound 1-66_P1. (Yield: 68%, MS: [M+H]+=489)

Compound 1-66_P1 (15 g, 30.7 mmol) and naphthalen-2-ylboronic acid (5.5 g, 32.2 mmol) were added to 300 ml of THF, and the mixture was stirred and refluxed. Then, potassium carbonate (12.7 g, 92 mmol) was dissolved in 38 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.6 g of Compound 1-66. (Yield: 71%, MS: [M+H]+=581)

### Preparation Example 1-67

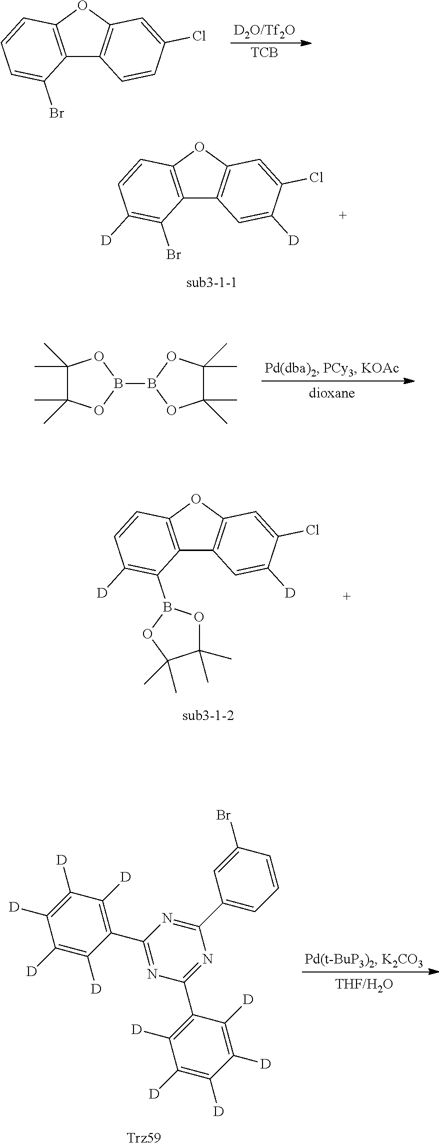

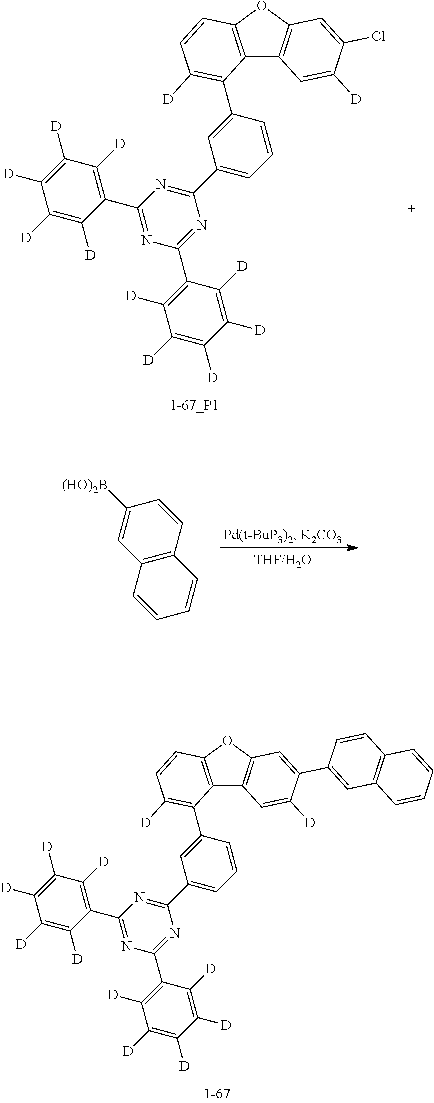 [SMILES: [2H]c1cc2c(cc1Cl)oc1ccc([2H])c(-c3cccc(-c4nc(-c5c([2H])c([2H])c([2H])c([2H])c5[2H])nc(-c5c([2H])c([2H])c([2H])c([2H])c5[2H])n4)c3)c12 ; OB(O)c1ccc2ccccc2c1 ; [2H]c1cc2c(cc1-c1ccc3ccccc3c1)oc1ccc([2H])c(-c3cccc(-c4nc(-c5c([2H])c([2H])c([2H])c([2H])c5[2H])nc(-c5c([2H])c([2H])c([2H])c([2H])c5[2H])n4)c3)c12]

Trifluoromethanesulfonic anhydride (30.1 g, 106.6 mmol) and deuterium oxide (10.7 g, 532.8 mmol) were added at 0° C. and stirred for 5 hours to prepare a solution. 1-bromo-7-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-7-chlorodibenzo[b,d]furan and 1,2,4-trichlorobenzene, and the mixture was stirred while heating to 140° C. and then keeping that temperature. After the reaction for 3 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6 g of sub3-1-1. (Yield: 40%, MS: [M+H]+=283)

Sub3-1-1 (15 g, 52.9 mmol) and bis(pinacolato)diboron (14.8 g, 58.2 mmol) were added to 300 ml of 1,4-dioxane, and the mixture was stirred and refluxed. Then, potassium acetate (7.8 g, 79.4 mmol) was added thereto, and the mixture was sufficiently stirred, and then bis(dibenzylideneacetone)palladium(0) (0.9 g, 1.6 mmol) and tricyclohexylphosphine (0.9 g, 3.2 mmol) were added. After the reaction for 4 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.4 g of sub3-1-2. (Yield: 65%, MS: [M+H]+=331)

Sub3-1-2 (15 g, 45.4 mmol) and Trz59 (19 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred, and then bis(tri-tert-butylphosphine)palladium(0) (0.2 g, 0.5 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 17.3 g of Compound 1-67_P1. (Yield: 73%, MS: [M+H]+=522)

Compound 1-67_P1 (15 g, 28.7 mmol) and naphthalen-2-ylboronic acid (5.2 g, 30.2 mmol) were added to 300 ml of THF, and the mixture was stirred and refluxed. Then, potassium carbonate (11.9 g, 86.2 mmol) was dissolved in 36 ml of water and added thereto, and the mixture was sufficiently stirred, and then bis(tri-tert-butylphosphine)palladium(0) (0.1 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.5 g of Compound 1-67. (Yield: 71%, MS: [M+H]+=614)

### Preparation Example 1-68

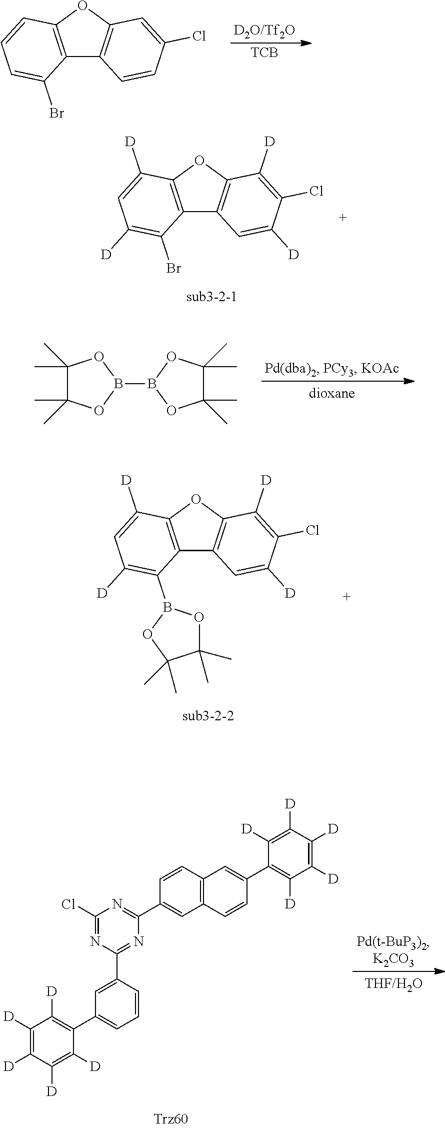

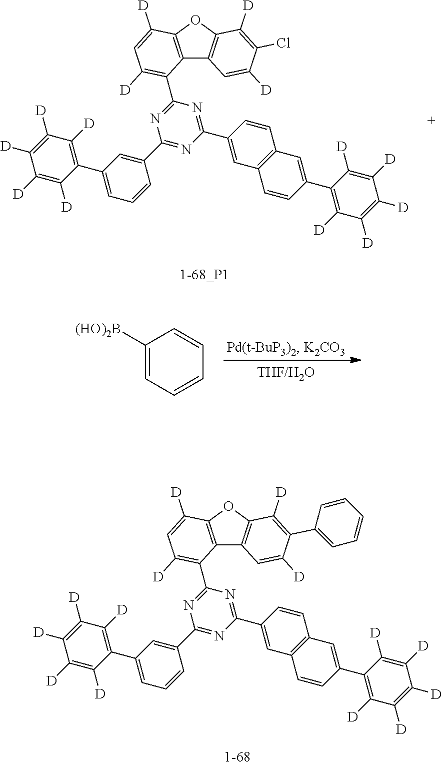 [SMILES: [2H]c1cc2c(oc3c([2H])cc([2H])c(-c4nc(-c5cccc(-c6c([2H])c([2H])c([2H])c([2H])c6[2H])c5)nc(-c5ccc6cc(-c7c([2H])c([2H])c([2H])c([2H])c7[2H])ccc6c5)n4)c32)c([2H])c1Cl ; OB(O)c1ccccc1 ; [2H]c1cc2c(oc3c([2H])cc([2H])c(-c4nc(-c5cccc(-c6c([2H])c([2H])c([2H])c([2H])c6[2H])c5)nc(-c5ccc6cc(-c7c([2H])c([2H])c([2H])c([2H])c7[2H])ccc6c5)n4)c32)c([2H])c1-c1ccccc1]

Trifluoromethanesulfonic anhydride (60.1 g, 213.1 mmol) and deuterium oxide (21.4 g, 1065.6 mmol) were added at 0° C. and stirred for 5 hours to prepare a solution. 1-Bromo-7-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-7-chlorodibenzo[b,d]furan and 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.7 g of sub3-2-1. (Yield: 44%, MS: [M+H]+=285)

Sub3-2-1 (15 g, 52.5 mmol) and bis(pinacolato)diboron (14.7 g, 57.8 mmol) were added to 300 ml of 1,4-dioxane, and the mixture was stirred and refluxed. Then, potassium acetate (7.7 g, 78.8 mmol) was added thereto, and the mixture was sufficiently stirred, and then bis(dibenzylideneacetone)palladium(0) (0.9 g, 1.6 mmol) and tricyclohexylphosphine (0.9 g, 3.2 mmol) were added. After the reaction for 6 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.7 g of sub3-2-2. (Yield: 67%, MS: [M+H]+=333)

Sub3-2-2 (15 g, 45.1 mmol) and Trz60 (22.7 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred, and then bis(tri-tert-butylphosphine)palladium(0) (0.2 g, 0.5 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 19.9 g of Compound 1-68_P1. (Yield: 68%, MS: [M+H]+=650)

Compound 1-68_P1 (15 g, 23.1 mmol) and phenylboronic acid (3 g, 24.2 mmol) were added to 300 ml of THF, and the mixture was stirred and refluxed. Then, potassium carbonate (9.6 g, 69.2 mmol) was dissolved in 29 ml of water and added thereto, and the mixture was sufficiently stirred, and then bis(tri-tert-butylphosphine)palladium(0) (0.1 g, 0.2 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.6 g of Compound 1-68. (Yield: 73%, MS: [M+H]+=692)

### Preparation Example 1-69

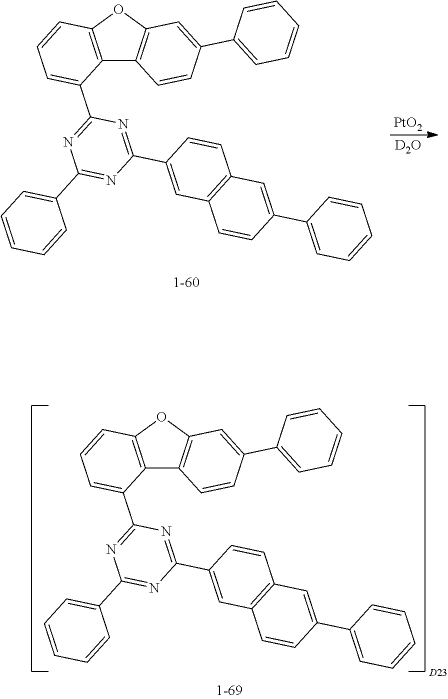

Compound 1-60 (10 g, 16.6 mmol), PtO₂ (1.1 g, 5 mmol) and D₂O (83 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 3.1 g of Compound 1-69. (Yield: 30%, MS: [M+H]+=626)

### Preparation Example 1-70

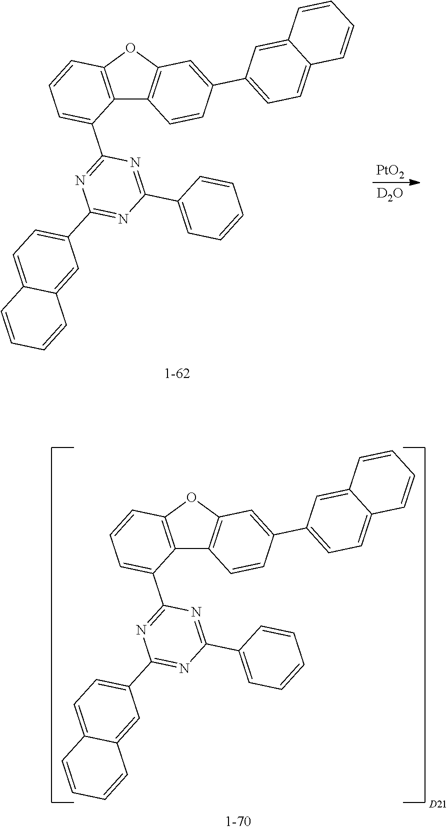

Compound 1-62 (10 g, 17.4 mmol), PtO₂ (1.2 g, 5.2 mmol) and D₂O (87 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 3.9 g of Compound 1-70. (Yield: 38%, MS: [M+H]+=598)

### Preparation Example 1-71

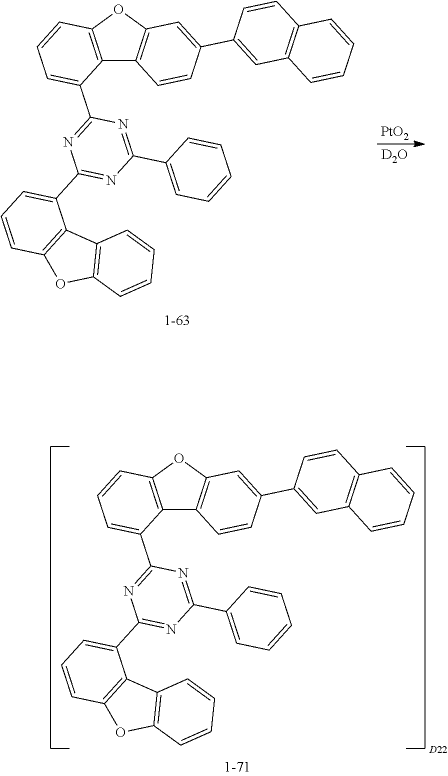

Compound 1-63 (10 g, 16.2 mmol), PtO₂ (1.1 g, 4.9 mmol) and D₂O (81 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 4.7 g of Compound 1-71. (Yield: 45%, MS: [M+H]+=639)

### Preparation Example 1-72

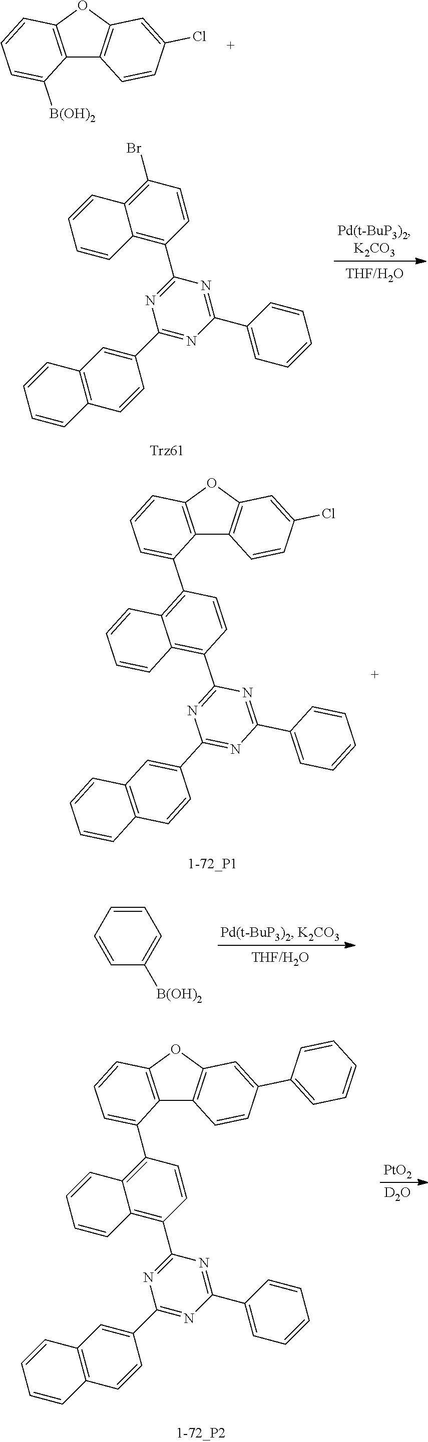

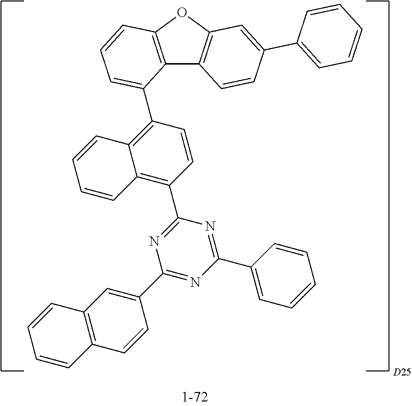

(7-Chlorodibenzo[b,d]furan-1-yl)boronic acid (15 g, 60.9 mmol) and Trz61 (31.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 24.5 g of Compound 1-72_P1. (Yield: 66%, MS: [M+H]+=610)

Compound 1-72_P1 (15 g, 24.6 mmol) and phenylboronic acid (3.1 g, 25.8 mmol) were added to 300 ml of THF, and the mixture was stirred and refluxed. Then, potassium carbonate (10.2 g, 73.8 mmol) was dissolved in 31 ml of water and added thereto, and the mixture was sufficiently stirred, and then bis(tri-tert-butylphosphine)palladium(0) (0.1 g, 0.2 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 10.6 g of Compound 1-72_P2. (Yield: 66%, MS: [M+H]+=652)

Compound 1-72_P2 (10 g, 15.3 mmol), PtO₂ (1 g, 4.6 mmol) and D₂O (77 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 4.6 g of Compound 1-72. (Yield: 44%, MS: [M+H]+=678)

### Preparation Example 1-73

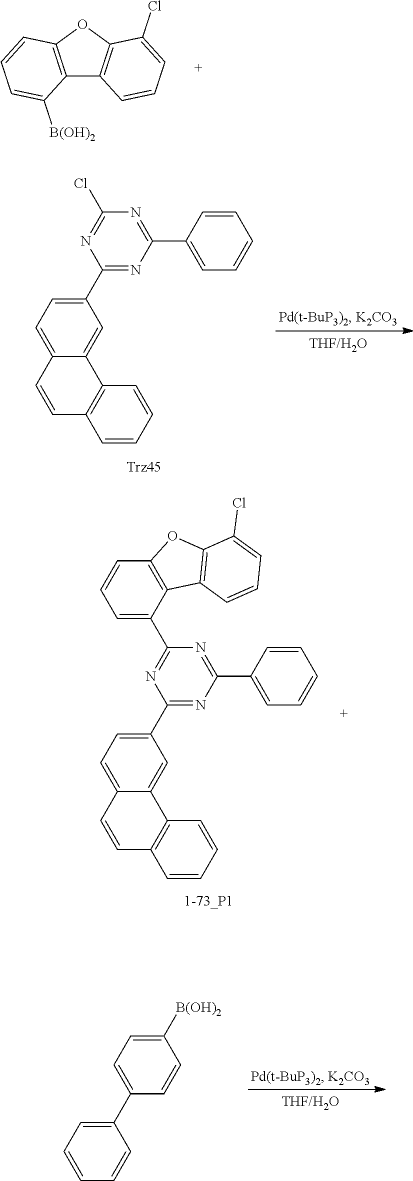 [SMILES: OB(O)c1cccc2oc3c(Cl)cccc3c12 ; Clc1nc(-c2ccccc2)nc(-c2ccc3ccc4ccccc4c3c2)n1 ; Clc1cccc2c1oc1cccc(-c3nc(-c4ccccc4)nc(-c4ccc5ccc6ccccc6c5c4)n3)c12 ; OB(O)c1ccc(-c2ccccc2)cc1]

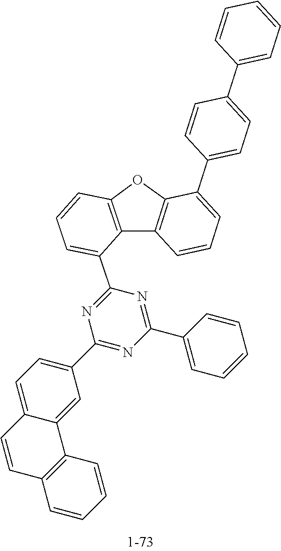 [SMILES: c1ccc(-c2ccc(-c3cccc4c3oc3cccc(-c5nc(-c6ccccc6)nc(-c6ccc7ccc8ccccc8c7c6)n5)c34)cc2)cc1]

(6-Chlorodibenzo[b,d]furan-1-yl)boronic acid (15 g, 60.9 mmol) and Trz45 (23.5 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 23.4 g of Compound 1-73_P1. (Yield: 72%, MS: [M+H]+=534)

Compound 1-73_P1 (15 g, 28.1 mmol) and [1,1′-biphenyl]-4-ylboronic acid (5.8 g, 29.5 mmol) were added to 300 ml of THF, and the mixture was stirred and refluxed. Then, potassium carbonate (11.6 g, 84.3 mmol) was dissolved in 35 ml of water and added thereto, and the mixture was sufficiently stirred, and then bis(tri-tert-butylphosphine)palladium(0) (0.1 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.4 g of Compound 1-73. (Yield: 73%, MS: [M+H]+=652)

### Preparation Example 1-74

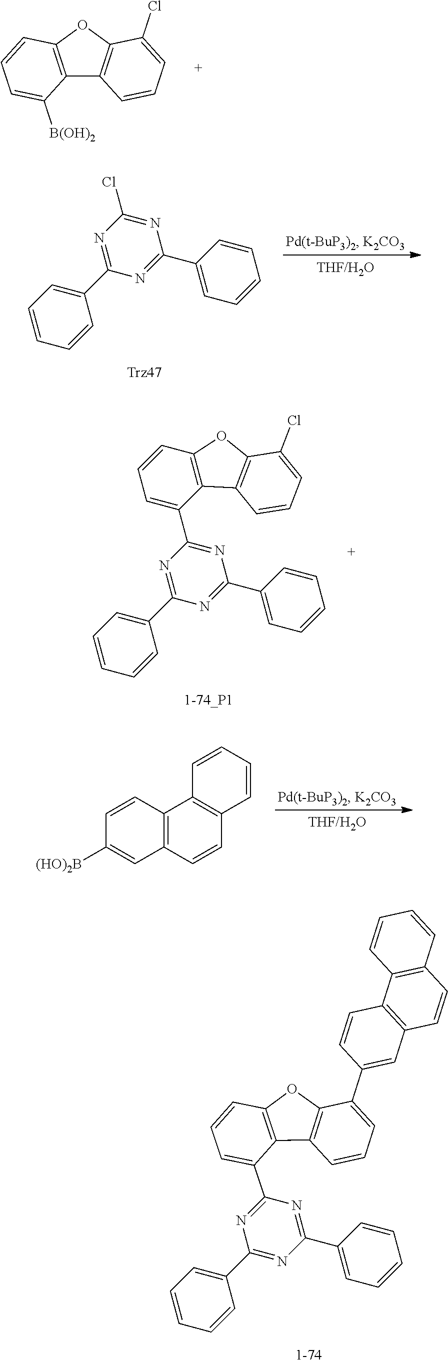 [SMILES: OB(O)c1cccc2oc3c(Cl)cccc3c12 ; Clc1nc(-c2ccccc2)nc(-c2ccccc2)n1 ; Clc1cccc2c1oc1cccc(-c3nc(-c4ccccc4)nc(-c4ccccc4)n3)c12 ; OB(O)c1ccc2c(ccc3ccccc32)c1 ; c1ccc(-c2nc(-c3ccccc3)nc(-c3cccc4oc5c(-c6ccc7c(ccc8ccccc87)c6)cccc5c34)n2)cc1]

(6-Chlorodibenzo[b,d]furan-1-yl)boronic acid (15 g, 60.9 mmol) and Trz47 (17.1 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 17.4 g of Compound 1-74_P1. (Yield: 66%, MS: [M+H]+=434)

Compound 1-74_P1 (15 g, 34.6 mmol) and phenanthren-2-ylboronic acid (8.1 g, 36.3 mmol) were added to 300 ml of THF, and the mixture was stirred and refluxed. Then, potassium carbonate (14.3 g, 103.7 mmol) was dissolved in 43 ml of water and added thereto, and the mixture was sufficiently stirred, and then bis(tri-tert-butylphosphine)palladium(0) (0.2 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.3 g of Compound 1-74. (Yield: 67%, MS: [M+H]+=576)

### Preparation Example 1-75

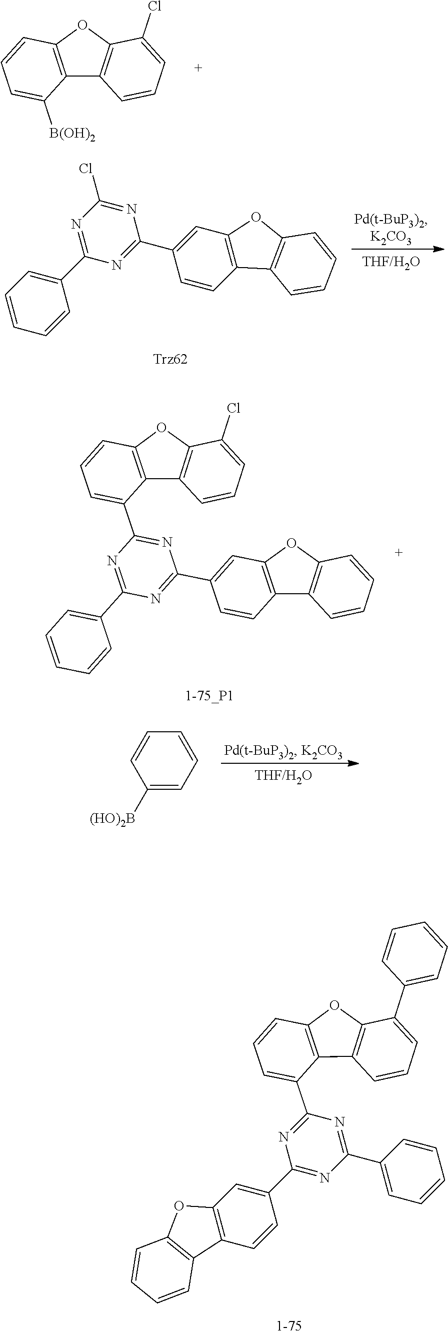 [SMILES: OB(O)c1cccc2oc3c(Cl)cccc3c12 ; Clc1nc(-c2ccccc2)nc(-c2ccc3c(c2)oc2ccccc23)n1 ; Clc1cccc2c1oc1cccc(-c3nc(-c4ccccc4)nc(-c4ccc5c(c4)oc4ccccc45)n3)c12 ; OB(O)c1ccccc1 ; c1ccc(-c2nc(-c3ccc4c(c3)oc3ccccc34)nc(-c3cccc4oc5c(-c6ccccc6)cccc5c34)n2)cc1]

(6-Chlorodibenzo[b,d]furan-1-yl)boronic acid (15 g, 60.9 mmol) and Trz62 (22.9 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 21.7 g of Compound 1-75_P1. (Yield: 68%, MS: [M+H]+=524)

Compound 1-75_P1 (15 g, 28.6 mmol) and phenylboronic acid (3.7 g, 30.1 mmol) were added to 300 ml of THF, and the mixture was stirred and refluxed. Then, potassium carbonate (11.9 g, 85.9 mmol) was dissolved in 36 ml of water and added thereto, and the mixture was sufficiently stirred, and then bis(tri-tert-butylphosphine)palladium(0) (0.1 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.8 g of Compound 1-75. (Yield: 73%, MS: [M+H]+=566)

### Preparation Example 1-76

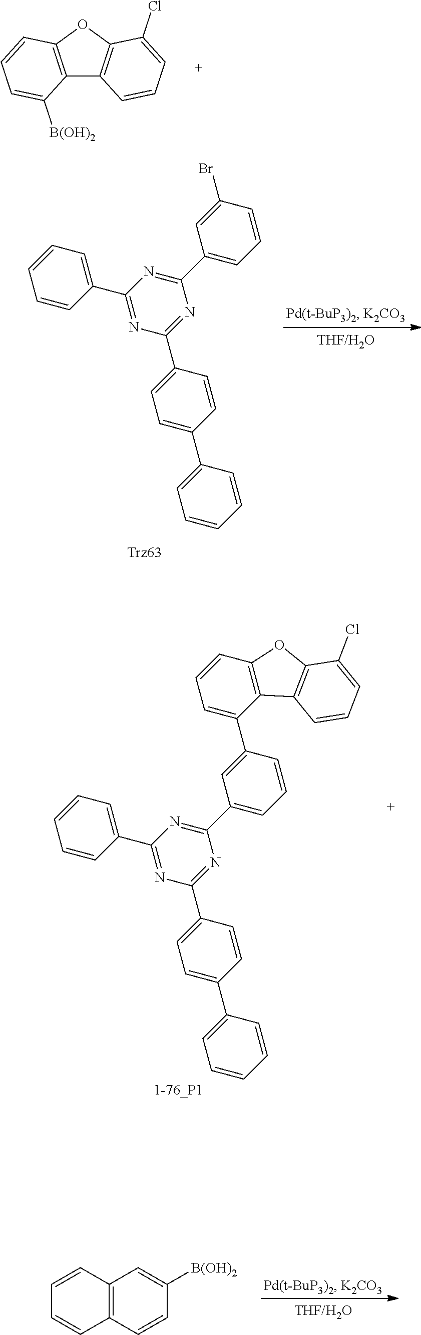 [SMILES: OB(O)c1cccc2oc3c(Cl)cccc3c12 ; Brc1cccc(-c2nc(-c3ccccc3)nc(-c3ccc(-c4ccccc4)cc3)n2)c1 ; Clc1cccc2c1oc1cccc(-c3cccc(-c4nc(-c5ccccc5)nc(-c5ccc(-c6ccccc6)cc5)n4)c3)c12 ; OB(O)c1ccc2ccccc2c1]

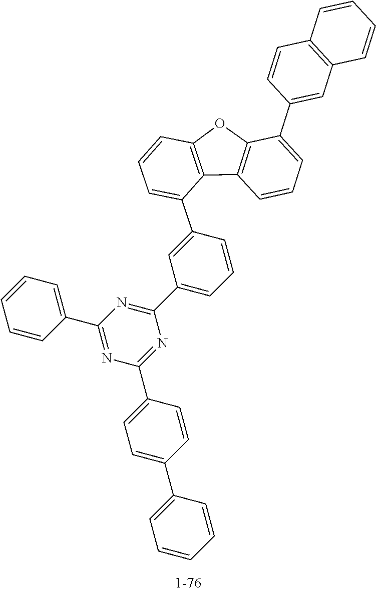 [SMILES: c1ccc(-c2ccc(-c3nc(-c4ccccc4)nc(-c4cccc(-c5cccc6oc7c(-c8ccc9ccccc9c8)cccc7c56)c4)n3)cc2)cc1]

(6-Chlorodibenzo[b,d]furan-1-yl)boronic acid (15 g, 60.9 mmol) and Trz63 (29.7 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 23.9 g of Compound 1-76_P1. (Yield: 67%, MS: [M+H]+=586)

Compound 1-76_P1 (15 g, 25.6 mmol) and naphthalen-2-ylboronic acid (4.6 g, 26.9 mmol) were added to 300 ml of THF, and the mixture was stirred and refluxed. Then, potassium carbonate (10.6 g, 76.8 mmol) was dissolved in 32 ml of water and added thereto, and the mixture was sufficiently stirred, and then bis(tri-tert-butylphosphine)palladium(0) (0.1 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.8 g of Compound 1-76. (Yield: 74%, MS: [M+H]+=678)

### Preparation Example 1-77

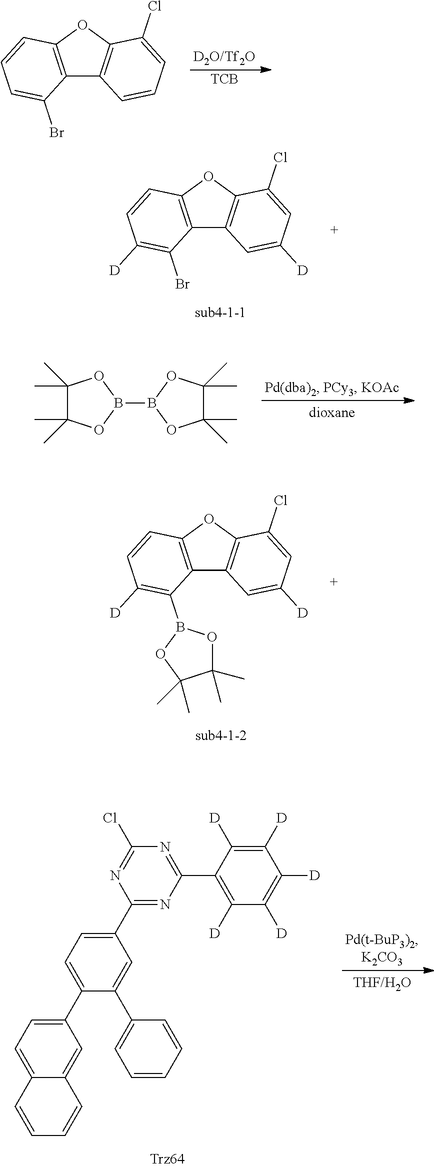

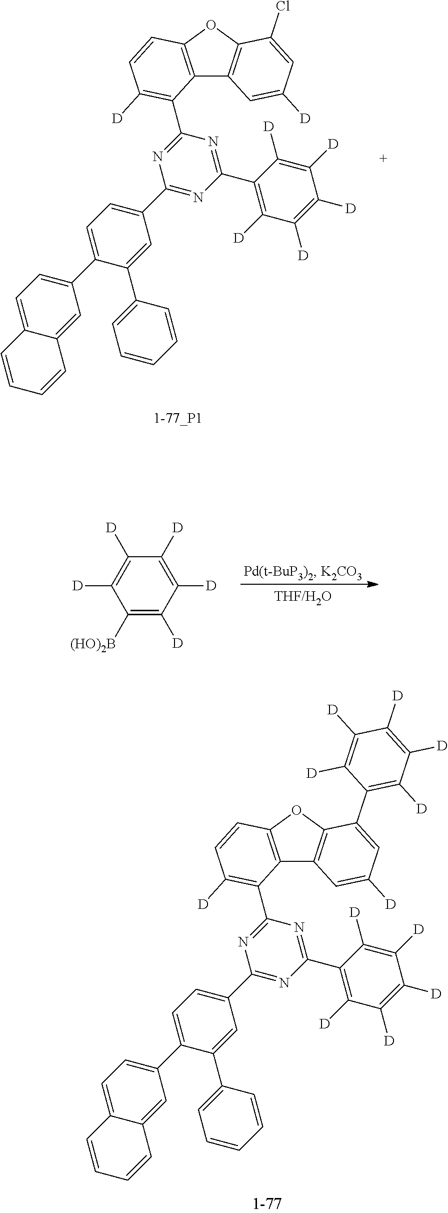 [SMILES: [2H]c1cc(Cl)c2oc3ccc([2H])c(-c4nc(-c5ccc(-c6ccc7ccccc7c6)c(-c6ccccc6)c5)nc(-c5c([2H])c([2H])c([2H])c([2H])c5[2H])n4)c3c2c1 ; [2H]c1c([2H])c([2H])c(B(O)O)c([2H])c1[2H] ; [2H]c1cc(-c2c([2H])c([2H])c([2H])c([2H])c2[2H])c2oc3ccc([2H])c(-c4nc(-c5ccc(-c6ccc7ccccc7c6)c(-c6ccccc6)c5)nc(-c5c([2H])c([2H])c([2H])c([2H])c5[2H])n4)c3c2c1]

Trifluoromethanesulfonic anhydride (30.1 g, 106.6 mmol) and deuterium oxide (10.7 g, 532.8 mmol) were added at 0° C. and stirred for 5 hours to prepare a solution. 1-Bromo-6-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-6-chlorodibenzo[b,d]furan and 1,2,4-trichlorobenzene, and the mixture was stirred while heating to 140° C. and then keeping that temperature. After the reaction for 3 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.8 g of sub4-1-1. (Yield: 45%, MS: [M+H]+=283)

Sub4-1-1 (15 g, 52.9 mmol) and bis(pinacolato)diboron (14.8 g, 58.2 mmol) were added to 300 ml of 1,4-dioxane, and the mixture was stirred and refluxed. Then, potassium acetate (7.8 g, 79.4 mmol) was added thereto, and the mixture was sufficiently stirred, and then bis(dibenzylideneacetone)palladium(0) (0.9 g, 1.6 mmol) and tricyclohexylphosphine (0.9 g, 3.2 mmol) were added. After the reaction for 6 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.1 g of sub4-1-2. (Yield: 75%, MS: [M+H]+=331)

Sub4-1-2 (15 g, 45.4 mmol) and Trz64 (22.6 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred, and then bis(tri-tert-butylphosphine)palladium(0) (0.2 g, 0.5 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 20.4 g of Compound 1-77_P1. (Yield: 70%, MS: [M+H]+=643)

Compound 1-77_P1 (15 g, 23.3 mmol) and (phenyl-d5)boronic acid (3.1 g, 24.5 mmol) were added to 300 ml of THF, and the mixture was stirred and refluxed. Then, potassium carbonate (9.7 g, 70 mmol) was dissolved in 29 ml of water and added thereto, and the mixture was sufficiently stirred, and then bis(tri-tert-butylphosphine)palladium(0) (0.1 g, 0.2 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.7 g of Compound 1-77. (Yield: 73%, MS: [M+H]+=690)

### Preparation Example 1-78

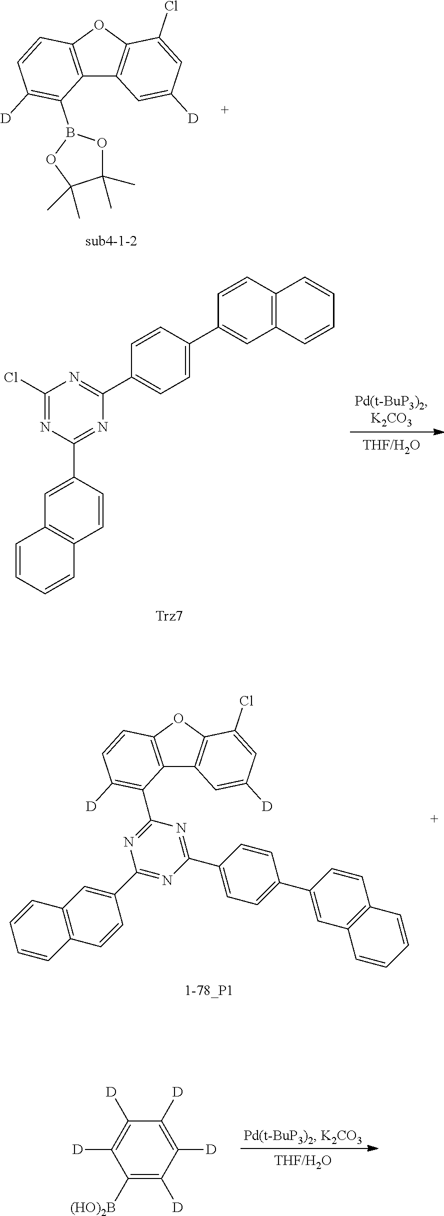 [SMILES: [2H]c1cc(Cl)c2oc3ccc([2H])c(B4OC(C)(C)C(C)(C)O4)c3c2c1 ; Clc1nc(-c2ccc(-c3ccc4ccccc4c3)cc2)nc(-c2ccc3ccccc3c2)n1 ; [2H]c1cc(Cl)c2oc3ccc([2H])c(-c4nc(-c5ccc(-c6ccc7ccccc7c6)cc5)nc(-c5ccc6ccccc6c5)n4)c3c2c1 ; [2H]c1c([2H])c([2H])c(B(O)O)c([2H])c1[2H]]

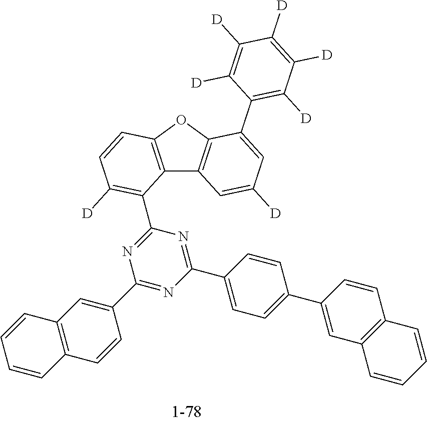 [SMILES: [2H]c1cc(-c2c([2H])c([2H])c([2H])c([2H])c2[2H])c2oc3ccc([2H])c(-c4nc(-c5ccc(-c6ccc7ccccc7c6)cc5)nc(-c5ccc6ccccc6c5)n4)c3c2c1]

Sub4-1-2 (15 g, 45.4 mmol) and Trz7 (21.1 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred, and then bis(tri-tert-butylphosphine)palladium(0) (0.2 g, 0.5 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 18 g of Compound 1-78_P1. (Yield: 65%, MS: [M+H]+=612)

Compound 1-78_P1 (15 g, 24.5 mmol) and (phenyl-d5)boronic acid (3.3 g, 25.7 mmol) were added to 300 ml of THF, and the mixture was stirred and refluxed. Then, potassium carbonate (10.2 g, 73.5 mmol) was dissolved in 30 ml of water and added thereto, and the mixture was sufficiently stirred, and then bis(tri-tert-butylphosphine)palladium(0) (0.1 g, 0.2 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.1 g of Compound 1-78. (Yield: 69%, MS: [M+H]+=659)

### Preparation Example 1-79

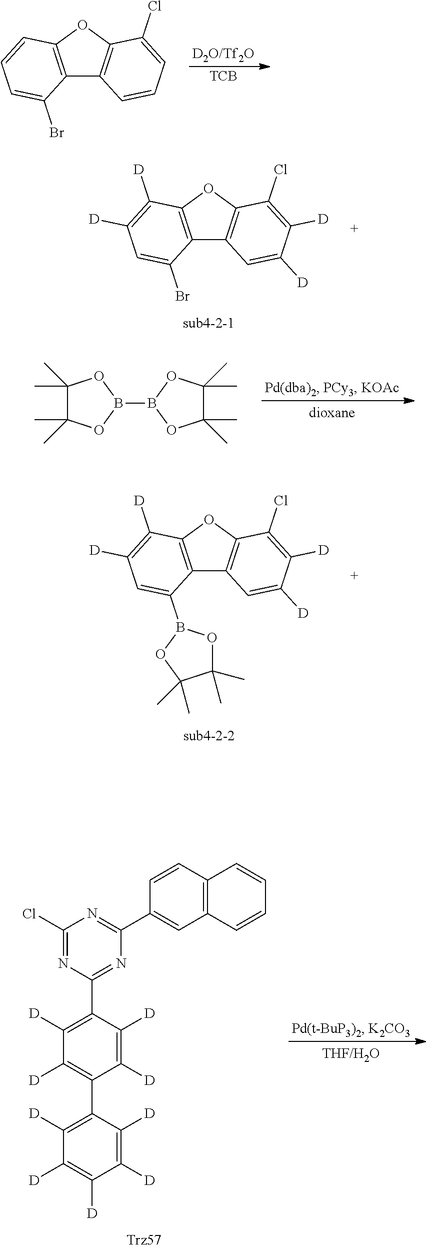

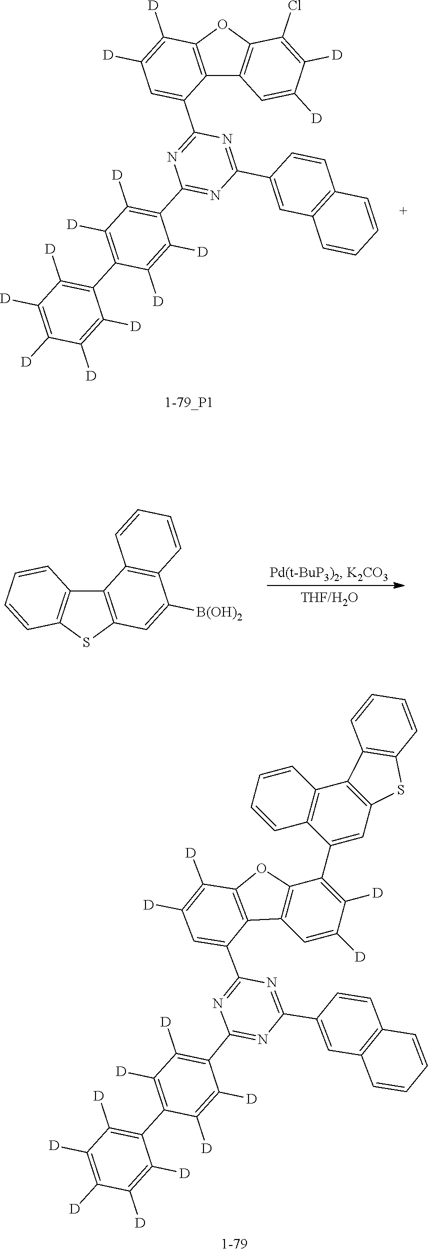 [SMILES: [2H]c1cc2c(oc3c([2H])c([2H])cc(-c4nc(-c5ccc6ccccc6c5)nc(-c5c([2H])c([2H])c(-c6c([2H])c([2H])c([2H])c([2H])c6[2H])c([2H])c5[2H])n4)c32)c(Cl)c1[2H] ; OB(O)c1cc2sc3ccccc3c2c2ccccc12 ; [2H]c1cc2c(oc3c([2H])c([2H])cc(-c4nc(-c5ccc6ccccc6c5)nc(-c5c([2H])c([2H])c(-c6c([2H])c([2H])c([2H])c([2H])c6[2H])c([2H])c5[2H])n4)c32)c(-c2cc3sc4ccccc4c3c3ccccc23)c1[2H]]

Trifluoromethanesulfonic anhydride (60.1 g, 213.1 mmol) and deuterium oxide (21.4 g, 1065.6 mmol) were added at 0° C. and stirred for 5 hours to prepare a solution. 1-Bromo-6-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-6-chlorodibenzo[b,d]furan and 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.5 g of sub4-2-1. (Yield: 43%, MS: [M+H]+=285)

Sub4-2-1 (15 g, 52.5 mmol) and bis(pinacolato)diboron (14.7 g, 57.8 mmol) were added to 300 ml of 1,4-dioxane, and the mixture was stirred and refluxed. Then, potassium acetate (7.7 g, 78.8 mmol) was added thereto, and the mixture was sufficiently stirred, and then bis(dibenzylideneacetone)palladium(0) (0.9 g, 1.6 mmol) and tricyclohexylphosphine (0.9 g, 3.2 mmol) were added. After the reaction for 5 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.1 g of sub4-2-2. (Yield: 75%, MS: [M+H]+=333)

Sub4-2-2 (15 g, 45.1 mmol) and Trz57 (19.1 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred, and then bis(tri-tert-butylphosphine)palladium(0) (0.2 g, 0.5 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 19.1 g of Compound 1-79_P1. (Yield: 74%, MS: [M+H]+=573)

Compound 1-79_P1 (15 g, 26.2 mmol) and benzo[b]naphtho[1,2-d]thiophen-5-ylboronic acid (7.6 g, 27.5 mmol) were added to 300 ml of THF, and the mixture was stirred and refluxed. Then, potassium carbonate (10.9 g, 78.5 mmol) was dissolved in 33 ml of water and added thereto, and the mixture was sufficiently stirred, and then bis(tri-tert-butylphosphine)palladium(0) (0.1 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.7 g of Compound 1-79. (Yield: 68%, MS: [M+H]+=771)

### Preparation Example 1-80

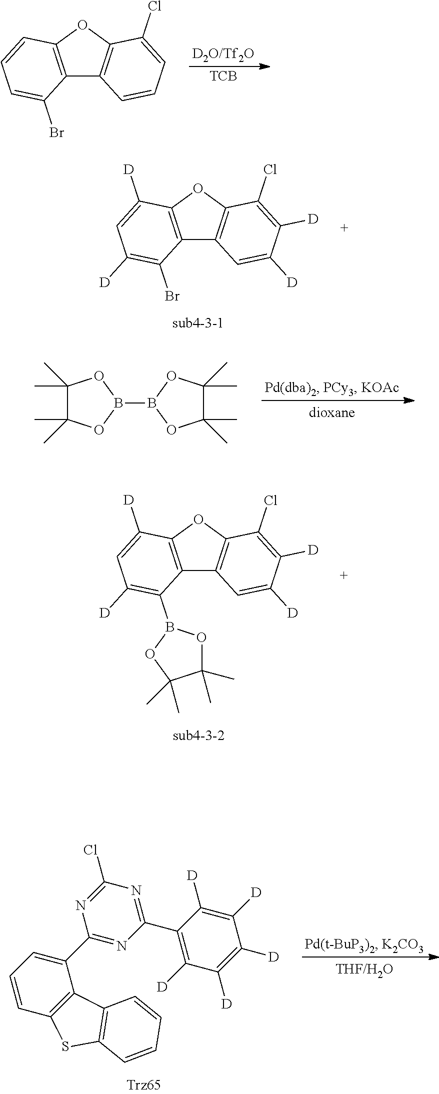

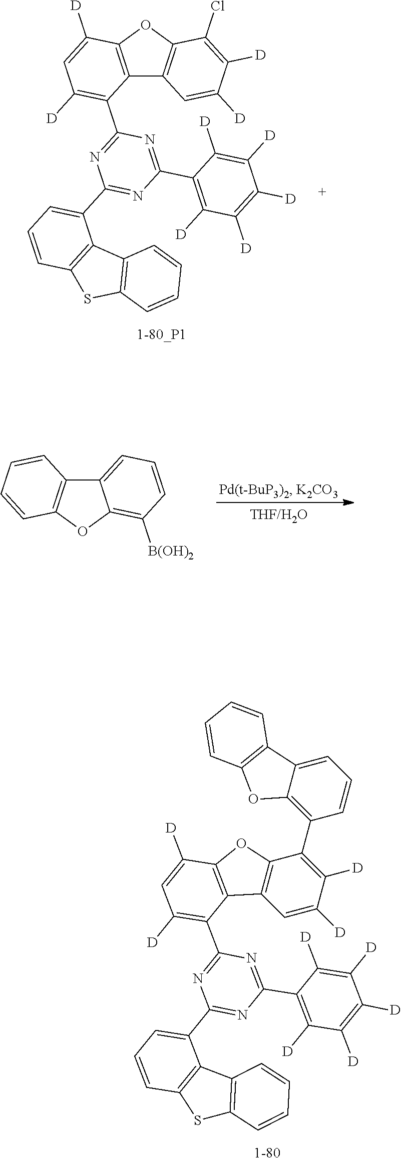 [SMILES: [2H]c1cc2c(oc3c([2H])cc([2H])c(-c4nc(-c5c([2H])c([2H])c([2H])c([2H])c5[2H])nc(-c5cccc6sc7ccccc7c56)n4)c32)c(Cl)c1[2H] ; OB(O)c1cccc2c1oc1ccccc12 ; [2H]c1cc2c(oc3c([2H])cc([2H])c(-c4nc(-c5c([2H])c([2H])c([2H])c([2H])c5[2H])nc(-c5cccc6sc7ccccc7c56)n4)c32)c(-c2cccc3c2oc2ccccc23)c1[2H]]

Trifluoromethanesulfonic anhydride (60.1 g, 213.1 mmol) and deuterium oxide (21.4 g, 1065.6 mmol) were added at 0° C. and stirred for 5 hours to prepare a solution. 1-Bromo-6-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-6-chlorodibenzo[b,d]furan and 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.4 g of sub4-3-1. (Yield: 42%, MS: [M+H]+=285)

Sub4-3-1 (15 g, 52.5 mmol) and bis(pinacolato)diboron (14.7 g, 57.8 mmol) were added to 300 ml of 1,4-dioxane, and the mixture was stirred and refluxed. Then, potassium acetate (7.7 g, 78.8 mmol) was added thereto, and the mixture was sufficiently stirred, and then bis(dibenzylideneacetone)palladium(0) (0.9 g, 1.6 mmol) and tricyclohexylphosphine (0.9 g, 3.2 mmol) were added. After the reaction for 6 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 10.8 g of sub4-3-2. (Yield: 62%, MS: [M+H]+=333)

Sub4-3-2 (15 g, 45.1 mmol) and Trz65 (17.9 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred, and then bis(tri-tert-butylphosphine)palladium(0) (0.2 g, 0.5 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 18 g of Compound 1-80_P1. (Yield: 73%, MS: [M+H]+=549)

Compound 1-80_P1 (15 g, 27.4 mmol) and dibenzo[b,d]furan-4-ylboronic acid (6.1 g, 28.7 mmol) were added to 300 ml of THF, and the mixture was stirred and refluxed. Then, potassium carbonate (11.3 g, 82.1 mmol) was dissolved in 34 ml of water and added thereto, and the mixture was sufficiently stirred, and then bis(tri-tert-butylphosphine)palladium(0) (0.1 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.3 g of Compound 1-80. (Yield: 66%, MS: [M+H]+=681)

### Preparation Example 1-81

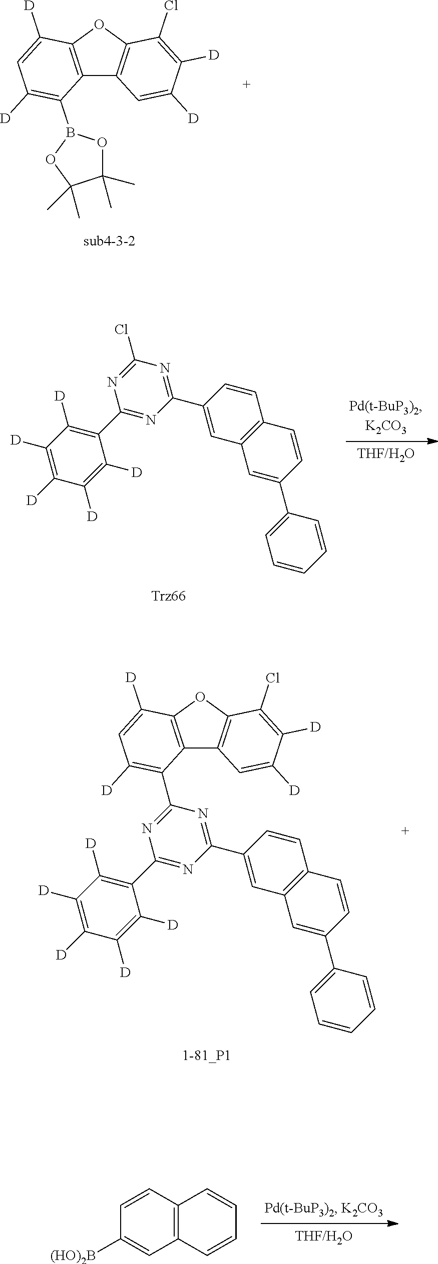 [SMILES: [2H]c1cc2c(oc3c([2H])cc([2H])c(B4OC(C)(C)C(C)(C)O4)c32)c(Cl)c1[2H] ; [2H]c1c([2H])c([2H])c(-c2nc(Cl)nc(-c3ccc4ccc(-c5ccccc5)cc4c3)n2)c([2H])c1[2H] ; [2H]c1cc2c(oc3c([2H])cc([2H])c(-c4nc(-c5ccc6ccc(-c7ccccc7)cc6c5)nc(-c5c([2H])c([2H])c([2H])c([2H])c5[2H])n4)c32)c(Cl)c1[2H] ; OB(O)c1ccc2ccccc2c1]

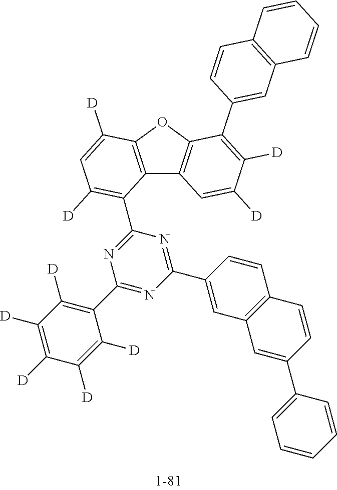 [SMILES: [2H]c1cc2c(oc3c([2H])cc([2H])c(-c4nc(-c5ccc6ccc(-c7ccccc7)cc6c5)nc(-c5c([2H])c([2H])c([2H])c([2H])c5[2H])n4)c32)c(-c2ccc3ccccc3c2)c1[2H]]

Sub4-3-2 (15 g, 45.1 mmol) and Trz66 (18.9 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred, and then bis(tri-tert-butylphosphine)palladium(0) (0.2 g, 0.5 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 19 g of Compound 1-81_P1. (Yield: 74%, MS: [M+H]+=569)

Compound 1-81_P1 (15 g, 26.4 mmol) and naphthalen-2-ylboronic acid (4.8 g, 27.7 mmol) were added to 300 ml of THF, and the mixture was stirred and refluxed. Then, potassium carbonate (10.9 g, 79.1 mmol) was dissolved in 33 ml of water and added thereto, and the mixture was sufficiently stirred, and then bis(tri-tert-butylphosphine)palladium(0) (0.1 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.5 g of Compound 1-81. (Yield: 72%, MS: [M+H]+=661)

### Preparation Example 1-82

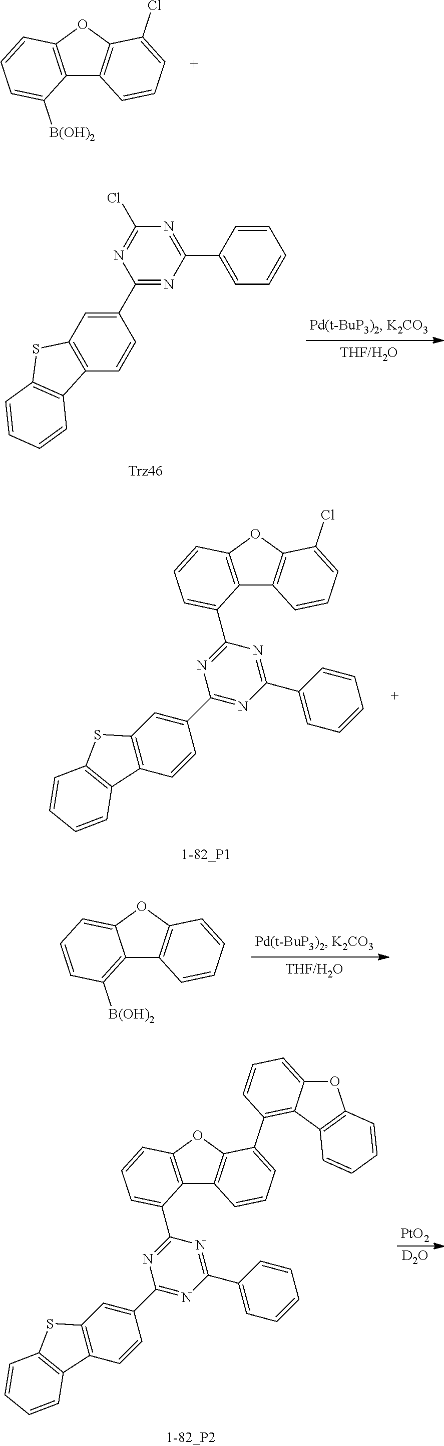

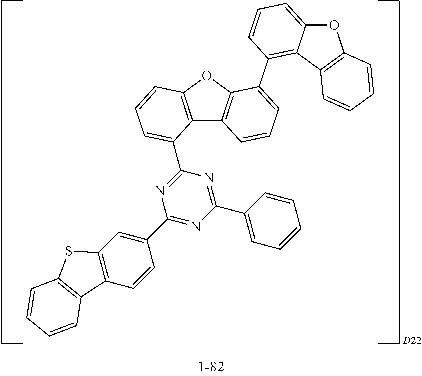

(6-Chlorodibenzo[b,d]furan-1-yl)boronic acid (15 g, 60.9 mmol) and Trz46 (23.9 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 23.3 g of Compound 1-82_P1. (Yield: 71%, MS: [M+H]+=540)

Compound 1-82_P1 (15 g, 23.3 mmol) and (phenyl-d5)boronic acid (3.1 g, 24.5 mmol) were added to 300 ml of THF, and the mixture was stirred and refluxed. Then, potassium carbonate (9.7 g, 70 mmol) was dissolved in 29 ml of water and added thereto, and the mixture was sufficiently stirred, and then bis(tri-tert-butylphosphine)palladium(0) (0.1 g, 0.2 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.3 g of Compound 1-82_P2. (Yield: 70%, MS: [M+H]+=690)

Compound 1-82_P2 (10 g, 14.9 mmol), PtO₂ (1 g, 4.5 mmol) and D₂O (74 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 3.8 g of Compound 1-82. (Yield: 37%, MS: [M+H]+=695)

### Preparation Example 1-83

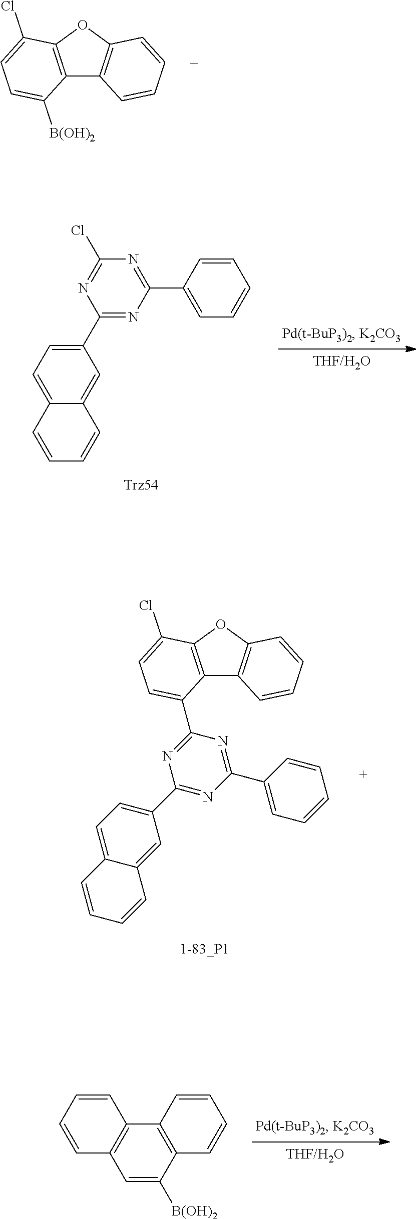 [SMILES: OB(O)c1ccc(Cl)c2oc3ccccc3c12 ; Clc1nc(-c2ccccc2)nc(-c2ccc3ccccc3c2)n1 ; Clc1ccc(-c2nc(-c3ccccc3)nc(-c3ccc4ccccc4c3)n2)c2c1oc1ccccc12 ; OB(O)c1cc2ccccc2c2ccccc12]

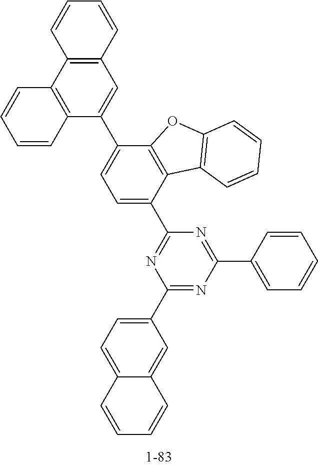 [SMILES: c1ccc(-c2nc(-c3ccc4ccccc4c3)nc(-c3ccc(-c4cc5ccccc5c5ccccc45)c4oc5ccccc5c34)n2)cc1]

(4-Chlorodibenzo[b,d]furan-1-yl)boronic acid (15 g, 60.9 mmol) and Trz54 (20.3 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 19.1 g of Compound 1-83_P1. (Yield: 65%, MS: [M+H]+=484)

Compound 1-83_P1 (15 g, 31 mmol) and phenanthren-9-ylboronic acid (7.2 g, 32.6 mmol) were added to 300 ml of THF, and the mixture was stirred and refluxed. Then, potassium carbonate (12.9 g, 93.1 mmol) was dissolved in 39 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 14.2 g of Compound 1-83. (Yield: 73%, MS: [M+H]+=626)

### Preparation Example 1-84

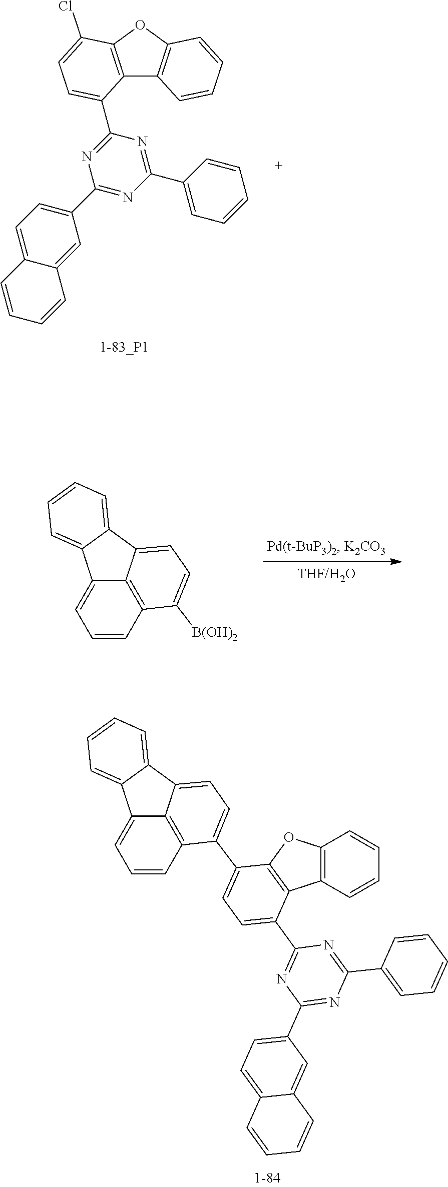 [SMILES: Clc1ccc(-c2nc(-c3ccccc3)nc(-c3ccc4ccccc4c3)n2)c2c1oc1ccccc12 ; OB(O)c1ccc2c3c(cccc13)-c1ccccc1-2 ; c1ccc(-c2nc(-c3ccc4ccccc4c3)nc(-c3ccc(-c4ccc5c6c(cccc46)-c4ccccc4-5)c4oc5ccccc5c34)n2)cc1]

Compound 1-83_P1 (15 g, 31 mmol) and fluoranthen-3-ylboronic acid (8 g, 32.6 mmol) were added to 300 ml of THF, and the mixture was stirred and refluxed. Then, potassium carbonate (12.9 g, 93.1 mmol) was dissolved in 39 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.3 g of Compound 1-84. (Yield: 66%, MS: [M+H]+=650)

### Preparation Example 1-85

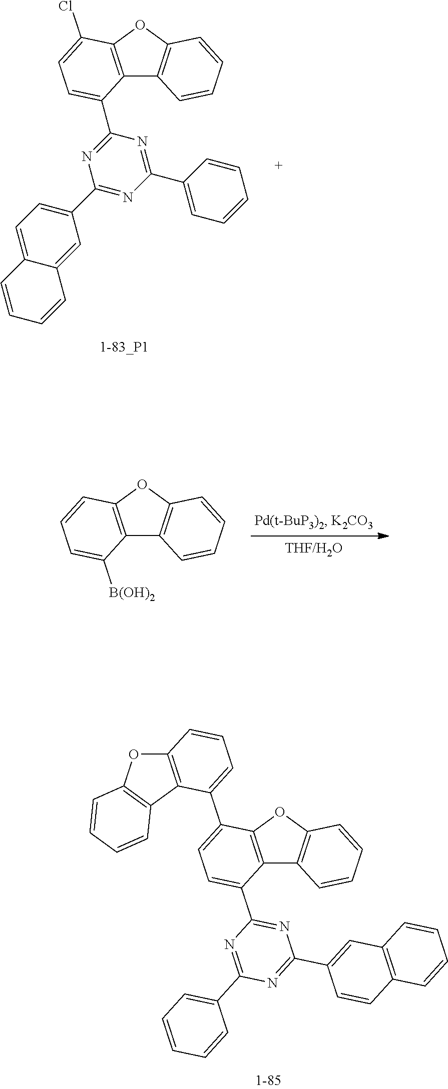 [SMILES: Clc1ccc(-c2nc(-c3ccccc3)nc(-c3ccc4ccccc4c3)n2)c2c1oc1ccccc12 ; OB(O)c1cccc2oc3ccccc3c12 ; c1ccc(-c2nc(-c3ccc4ccccc4c3)nc(-c3ccc(-c4cccc5oc6ccccc6c45)c4oc5ccccc5c34)n2)cc1]

Compound 1-83_P1 (15 g, 31 mmol) and dibenzo[b,d]furan-1-ylboronic acid (6.9 g, 32.6 mmol) were added to 300 ml of THF, and the mixture was stirred and refluxed. Then, potassium carbonate (12.9 g, 93.1 mmol) was dissolved in 39 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.8 g of Compound 1-85. (Yield: 72%, MS: [M+H]+=616)

### Preparation Example 1-86

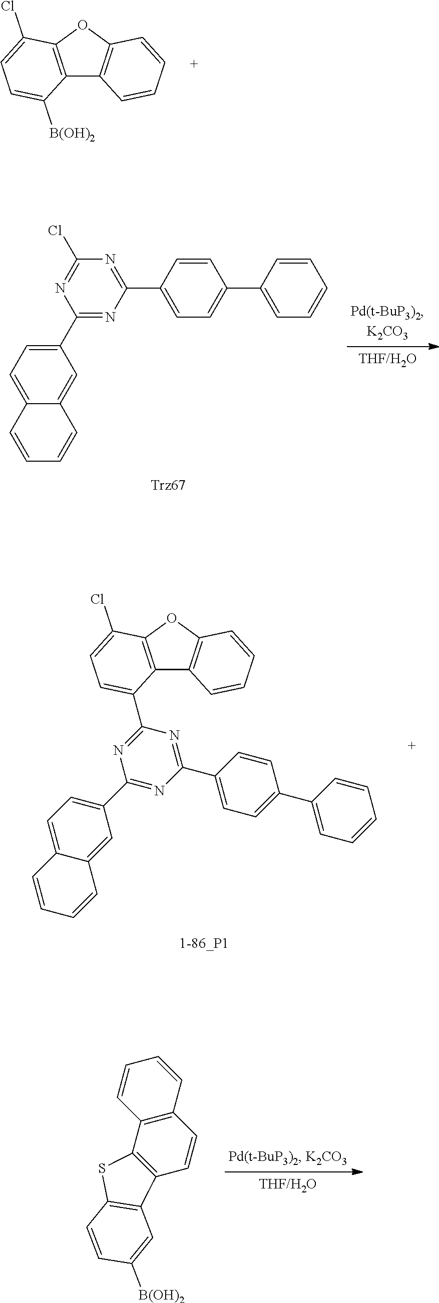 [SMILES: OB(O)c1ccc(Cl)c2oc3ccccc3c12 ; Clc1nc(-c2ccc(-c3ccccc3)cc2)nc(-c2ccc3ccccc3c2)n1 ; Clc1ccc(-c2nc(-c3ccc(-c4ccccc4)cc3)nc(-c3ccc4ccccc4c3)n2)c2c1oc1ccccc12 ; OB(O)c1ccc2sc3c4ccccc4ccc3c2c1]

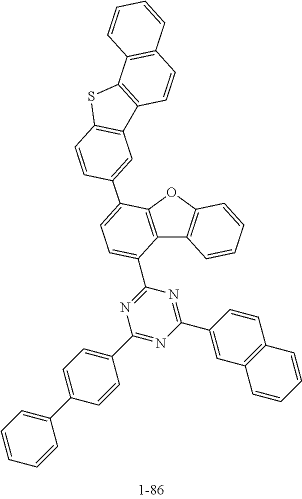 [SMILES: c1ccc(-c2ccc(-c3nc(-c4ccc5ccccc5c4)nc(-c4ccc(-c5ccc6sc7c8ccccc8ccc7c6c5)c5oc6ccccc6c45)n3)cc2)cc1]

(4-Chlorodibenzo[b,d]furan-1-yl)boronic acid (15 g, 60.9 mmol) and Trz67 (25.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 25.5 g of Compound 1-86_P1. (Yield: 75%, MS: [M+H]+=560)

Compound 1-86_P1 (15 g, 26.8 mmol) and benzo[b]naphtho[2,1-d]thiophen-8-ylboronic acid (7.8 g, 28.1 mmol) were added to 300 ml of THF, and the mixture was stirred and refluxed. Then, potassium carbonate (11.1 g, 80.3 mmol) was dissolved in 33 ml of water and added thereto, and the mixture was sufficiently stirred, and then bis(tri-tert-butylphosphine)palladium(0) (0.1 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.4 g of Compound 1-86. (Yield: 66%, MS: [M+H]+=758)

### Preparation Example 1-87

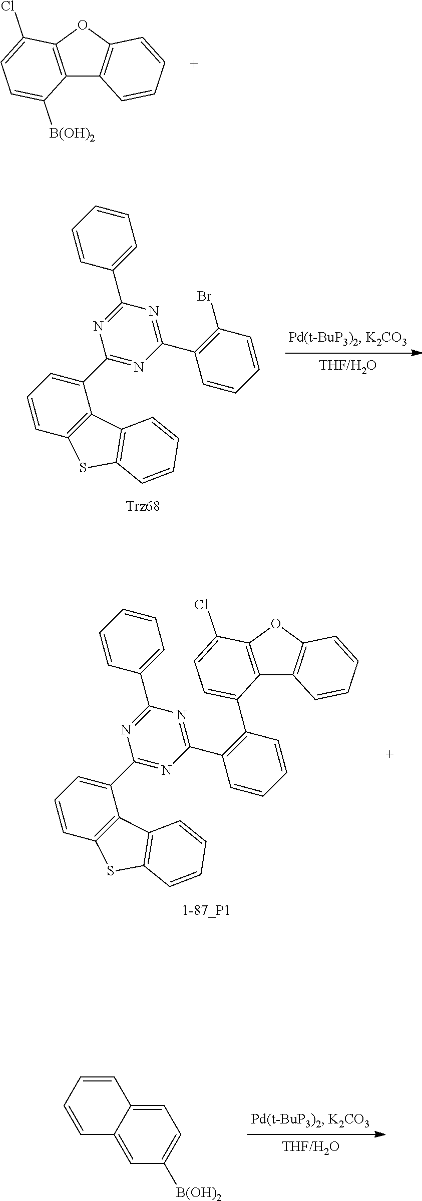 [SMILES: OB(O)c1ccc(Cl)c2oc3ccccc3c12 ; Brc1ccccc1-c1nc(-c2ccccc2)nc(-c2cccc3sc4ccccc4c23)n1 ; Clc1ccc(-c2ccccc2-c2nc(-c3ccccc3)nc(-c3cccc4sc5ccccc5c34)n2)c2c1oc1ccccc12 ; OB(O)c1ccc2ccccc2c1]

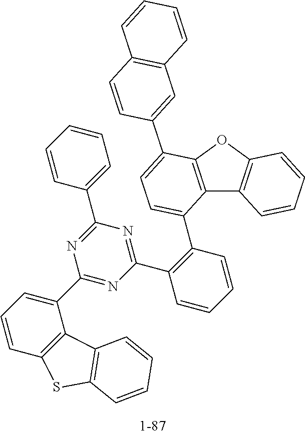 [SMILES: c1ccc(-c2nc(-c3ccccc3-c3ccc(-c4ccc5ccccc5c4)c4oc5ccccc5c34)nc(-c3cccc4sc5ccccc5c34)n2)cc1]

(4-Chlorodibenzo[b,d]furan-1-yl)boronic acid (15 g, 60.9 mmol) and Trz68 (31.6 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 27.7 g of Compound 1-87_P1. (Yield: 74%, MS: [M+H]+=616)

Compound 1-87_P1 (15 g, 24.3 mmol) and naphthalen-2-ylboronic acid (4.4 g, 25.6 mmol) were added to 300 ml of THF, and the mixture was stirred and refluxed. Then, potassium carbonate (10.1 g, 73 mmol) was dissolved in 30 ml of water and added thereto, and the mixture was sufficiently stirred, and then bis(tri-tert-butylphosphine)palladium(0) (0.1 g, 0.2 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.4 g of Compound 1-87. (Yield: 72%, MS: [M+H]+=708)

### Preparation Example 1-88

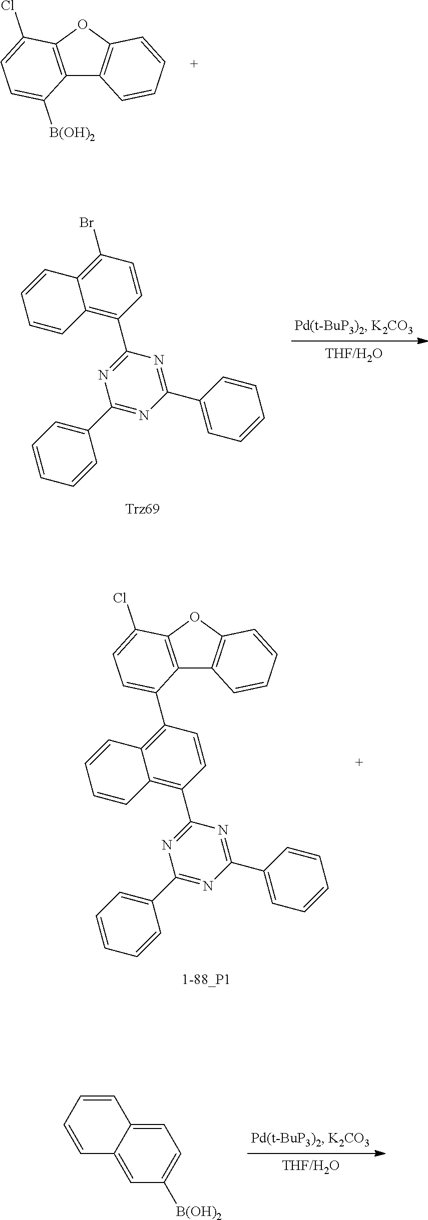 [SMILES: OB(O)c1ccc(Cl)c2oc3ccccc3c12 ; Brc1ccc(-c2nc(-c3ccccc3)nc(-c3ccccc3)n2)c2ccccc12 ; Clc1ccc(-c2ccc(-c3nc(-c4ccccc4)nc(-c4ccccc4)n3)c3ccccc23)c2c1oc1ccccc12 ; OB(O)c1ccc2ccccc2c1]

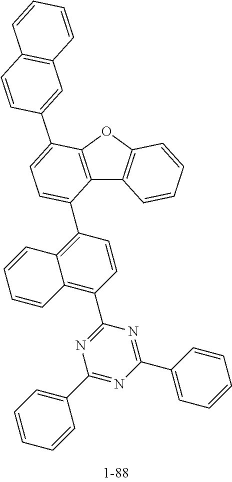 [SMILES: c1ccc(-c2nc(-c3ccccc3)nc(-c3ccc(-c4ccc(-c5ccc6ccccc6c5)c5oc6ccccc6c45)c4ccccc34)n2)cc1]

(4-Chlorodibenzo[b,d]furan-1-yl)boronic acid (15 g, 60.9 mmol) and Trz69 (28 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 24.2 g of Compound 1-88_P1. (Yield: 71%, MS: [M+H]+=560)

Compound 1-88_P1 (15 g, 26.8 mmol) and naphthalen-2-ylboronic acid (4.8 g, 28.1 mmol) were added to 300 ml of THF, and the mixture was stirred and refluxed. Then, potassium carbonate (11.1 g, 80.3 mmol) was dissolved in 33 ml of water and added thereto, and the mixture was sufficiently stirred, and then bis(tri-tert-butylphosphine)palladium(0) (0.1 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.5 g of Compound 1-88. (Yield: 66%, MS: [M+H]+=652)

### Preparation Example 1-89

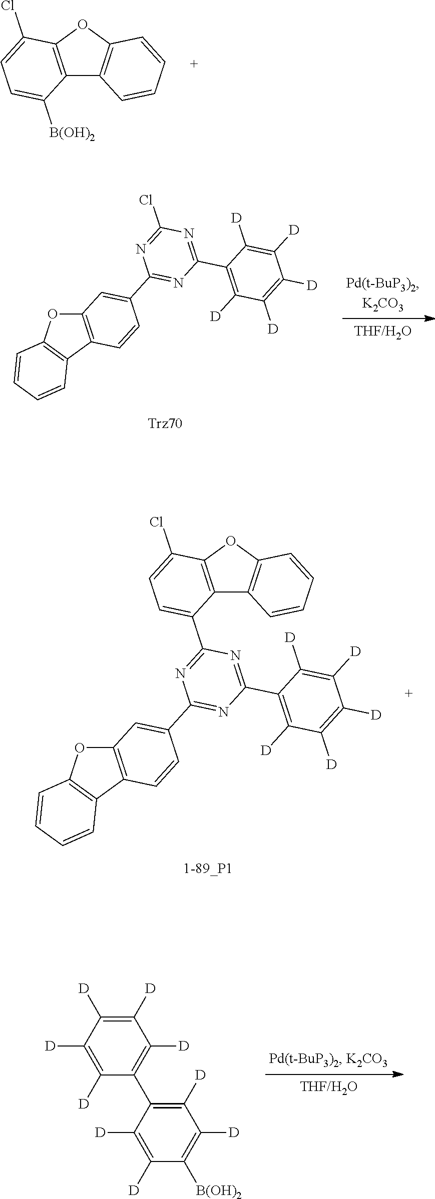 [SMILES: OB(O)c1ccc(Cl)c2oc3ccccc3c12 ; [2H]c1c([2H])c([2H])c(-c2nc(Cl)nc(-c3ccc4c(c3)oc3ccccc34)n2)c([2H])c1[2H] ; [2H]c1c([2H])c([2H])c(-c2nc(-c3ccc4c(c3)oc3ccccc34)nc(-c3ccc(Cl)c4oc5ccccc5c34)n2)c([2H])c1[2H] ; [2H]c1c([2H])c([2H])c(-c2c([2H])c([2H])c(B(O)O)c([2H])c2[2H])c([2H])c1[2H]]

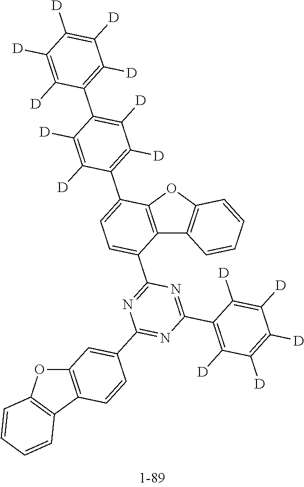 [SMILES: [2H]c1c([2H])c([2H])c(-c2nc(-c3ccc4c(c3)oc3ccccc34)nc(-c3ccc(-c4c([2H])c([2H])c(-c5c([2H])c([2H])c([2H])c([2H])c5[2H])c([2H])c4[2H])c4oc5ccccc5c34)n2)c([2H])c1[2H]]

(4-Chlorodibenzo[b,d]furan-1-yl)boronic acid (15 g, 60.9 mmol) and Trz70 (23.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 21.5 g of Compound 1-89_P1. (Yield: 67%, MS: [M+H]+=529)

Compound 1-89_P1 (15 g, 28.4 mmol) and ([1,1′-biphenyl]-4-yl-d9)boronic acid (6.2 g, 29.8 mmol) were added to 300 ml of THF, and the mixture was stirred and refluxed. Then, potassium carbonate (11.8 g, 85.1 mmol) was dissolved in 35 ml of water and added thereto, and the mixture was sufficiently stirred, and then bis(tri-tert-butylphosphine)palladium(0) (0.1 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.3 g of Compound 1-89. (Yield: 66%, MS: [M+H]+=656)

### Preparation Example 1-90

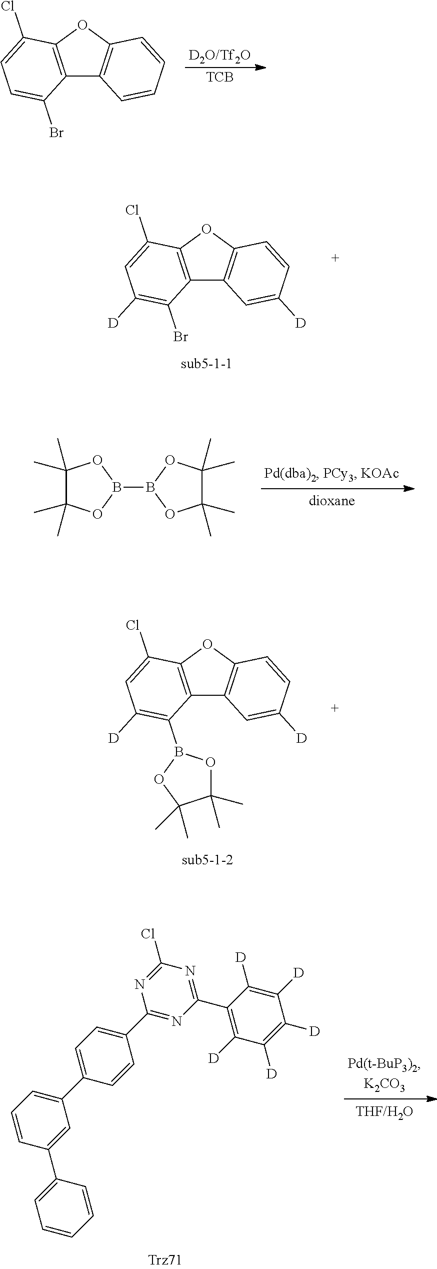

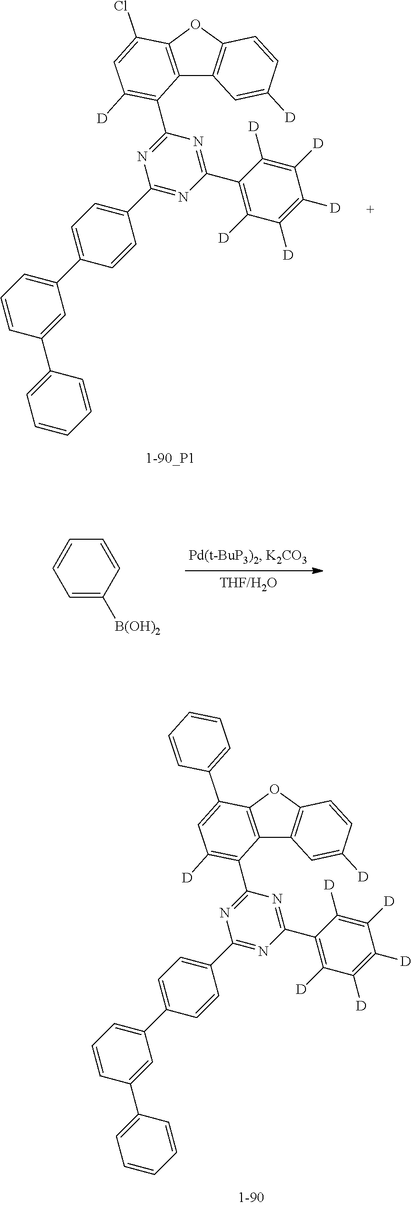 [SMILES: [2H]c1ccc2oc3c(Cl)cc([2H])c(-c4nc(-c5ccc(-c6cccc(-c7ccccc7)c6)cc5)nc(-c5c([2H])c([2H])c([2H])c([2H])c5[2H])n4)c3c2c1 ; OB(O)c1ccccc1 ; [2H]c1ccc2oc3c(-c4ccccc4)cc([2H])c(-c4nc(-c5ccc(-c6cccc(-c7ccccc7)c6)cc5)nc(-c5c([2H])c([2H])c([2H])c([2H])c5[2H])n4)c3c2c1]

Trifluoromethanesulfonic anhydride (30.1 g, 106.6 mmol) and deuterium oxide (10.7 g, 532.8 mmol) were added at 0° C. and stirred for 5 hours to prepare a solution. 1-Bromo-4-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-4-chlorodibenzo[b,d]furan and 1,2,4-trichlorobenzene, and the mixture was stirred while heating to 140° C. and then keeping that temperature. After the reaction for 3 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.5 g of sub5-1-1. (Yield: 43%, MS: [M+H]+=283)

Sub5-1-1 (15 g, 52.9 mmol) and bis(pinacolato)diboron (14.8 g, 58.2 mmol) were added to 300 ml of 1,4-dioxane, and the mixture was stirred and refluxed. Then, potassium acetate (7.8 g, 79.4 mmol) was added thereto, and the mixture was sufficiently stirred, and then bis(dibenzylideneacetone)palladium(0) (0.9 g, 1.6 mmol) and tricyclohexylphosphine (0.9 g, 3.2 mmol) were added. After the reaction for 6 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 10.8 g of sub5-1-2. (Yield: 62%, MS: [M+H]+=331)

Sub5-1-2 (15 g, 45.4 mmol) and Trz71 (20.2 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred, and then bis(tri-tert-butylphosphine)palladium(0) (0.2 g, 0.5 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 19.9 g of Compound 1-90_P1. (Yield: 74%, MS: [M+H]+=594)

Compound 1-90_P1 (15 g, 25.3 mmol) and phenylboronic acid (3.2 g, 26.6 mmol) were added to 300 ml of THF, and the mixture was stirred and refluxed. Then, potassium carbonate (10.5 g, 75.9 mmol) was dissolved in 31 ml of water and added thereto, and the mixture was sufficiently stirred, and then bis(tri-tert-butylphosphine)palladium(0) (0.1 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.7 g of Compound 1-90. (Yield: 73%, MS: [M+H]+=635)

### Preparation Example 1-91

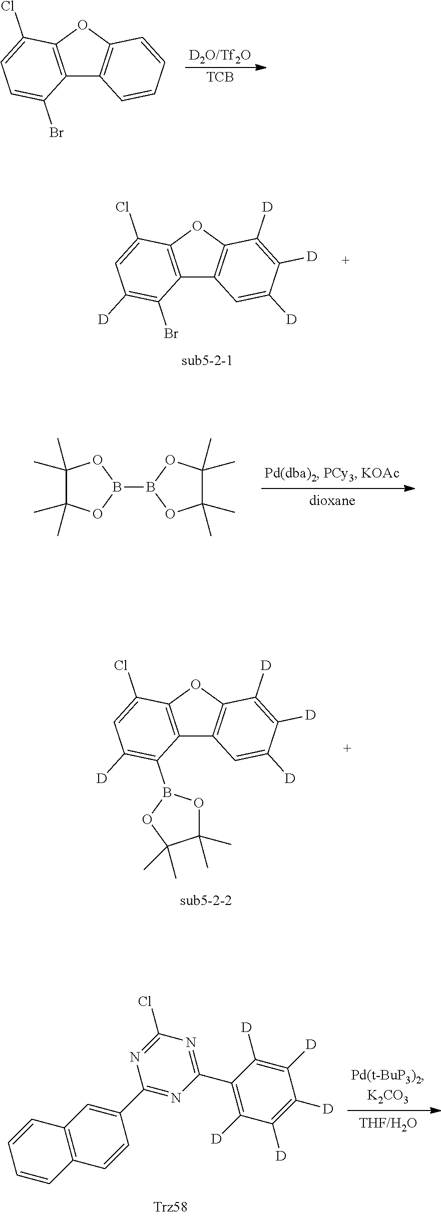

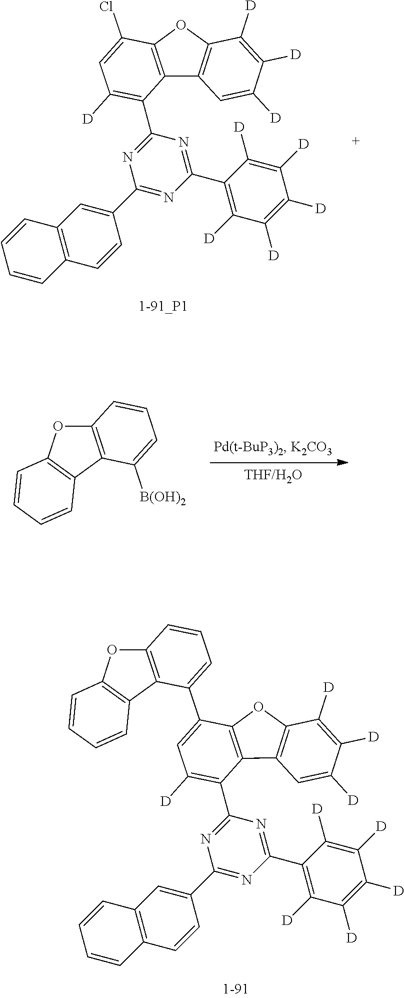 [SMILES: [2H]c1cc2c(oc3c(Cl)cc([2H])c(-c4nc(-c5ccc6ccccc6c5)nc(-c5c([2H])c([2H])c([2H])c([2H])c5[2H])n4)c32)c([2H])c1[2H] ; OB(O)c1cccc2oc3ccccc3c12 ; [2H]c1cc2c(oc3c(-c4cccc5oc6ccccc6c45)cc([2H])c(-c4nc(-c5ccc6ccccc6c5)nc(-c5c([2H])c([2H])c([2H])c([2H])c5[2H])n4)c32)c([2H])c1[2H]]

Trifluoromethanesulfonic anhydride (60.1 g, 213.1 mmol) and deuterium oxide (21.4 g, 1065.6 mmol) were added at 0° C. and stirred for 5 hours to prepare a solution. 1-Bromo-4-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-4-chlorodibenzo[b,d]furan and 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.3 g of sub5-2-1. (Yield: 35%, MS: [M+H]+=285)

Sub5-2-1 (15 g, 52.5 mmol) and bis(pinacolato)diboron (14.7 g, 57.8 mmol) were added to 300 ml of 1,4-dioxane, and the mixture was stirred and refluxed. Then, potassium acetate (7.7 g, 78.8 mmol) was added thereto, and the mixture was sufficiently stirred, and then bis(dibenzylideneacetone)palladium(0) (0.9 g, 1.6 mmol) and tricyclohexylphosphine (0.9 g, 3.2 mmol) were added. After the reaction for 6 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11 g of sub5-2-2. (Yield: 63%, MS: [M+H]+=333)

Sub5-2-2 (15 g, 45.1 mmol) and Trz58 (15.8 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred, and then bis(tri-tert-butylphosphine)palladium(0) (0.2 g, 0.5 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 16.6 g of Compound 1-91_P1. (Yield: 75%, MS: [M+H]+=493)

Compound 1-91_P1 (15 g, 30.4 mmol) and dibenzo[b,d]furan-1-ylboronic acid (6.8 g, 31.9 mmol) were added to 300 ml of THF, and the mixture was stirred and refluxed. Then, potassium carbonate (12.6 g, 91.3 mmol) was dissolved in 38 ml of water and added thereto, and the mixture was sufficiently stirred, and then bis(tri-tert-butylphosphine)palladium(0) (0.2 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.1 g of Compound 1-91. (Yield: 69%, MS: [M+H]+=625)

### Preparation Example 1-92

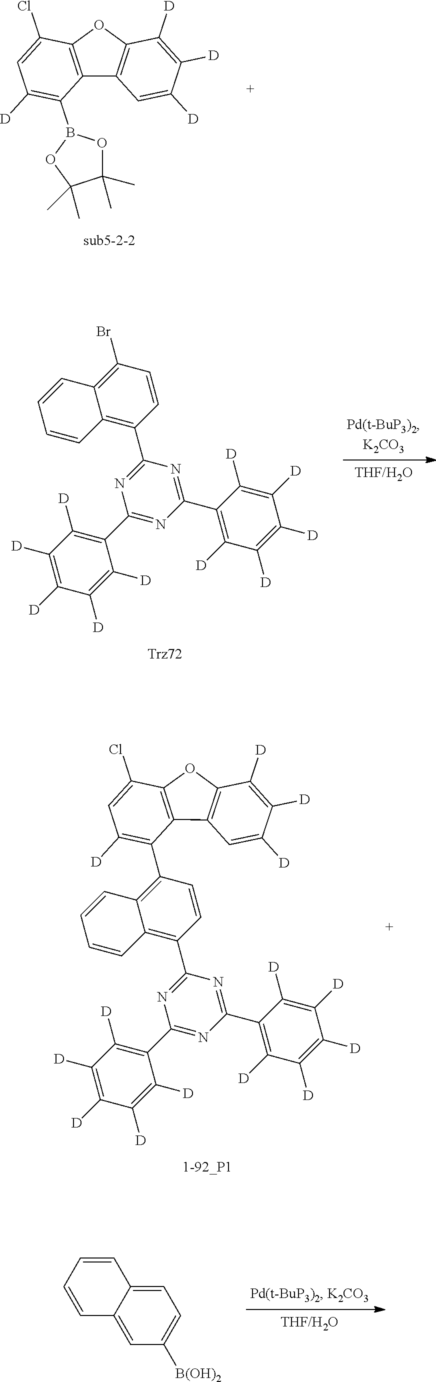 [SMILES: [2H]c1cc2c(oc3c(Cl)cc([2H])c(B4OC(C)(C)C(C)(C)O4)c32)c([2H])c1[2H] ; [2H]c1c([2H])c([2H])c(-c2nc(-c3c([2H])c([2H])c([2H])c([2H])c3[2H])nc(-c3ccc(Br)c4ccccc34)n2)c([2H])c1[2H] ; [2H]c1cc2c(oc3c(Cl)cc([2H])c(-c4ccc(-c5nc(-c6c([2H])c([2H])c([2H])c([2H])c6[2H])nc(-c6c([2H])c([2H])c([2H])c([2H])c6[2H])n5)c5ccccc45)c32)c([2H])c1[2H] ; OB(O)c1ccc2ccccc2c1]

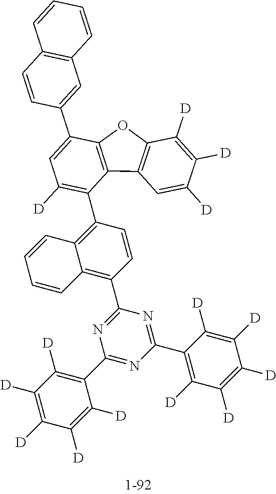 [SMILES: [2H]c1cc2c(oc3c(-c4ccc5ccccc5c4)cc([2H])c(-c4ccc(-c5nc(-c6c([2H])c([2H])c([2H])c([2H])c6[2H])nc(-c6c([2H])c([2H])c([2H])c([2H])c6[2H])n5)c5ccccc45)c32)c([2H])c1[2H]]

Sub5-2-2 (15 g, 45.1 mmol) and Trz72 (21.2 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred, and then bis(tri-tert-butylphosphine)palladium(0) (0.2 g, 0.5 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 18.4 g of Compound 1-92_P1. (Yield: 71%, MS: [M+H]+=574)

Compound 1-92_P1 (15 g, 26.1 mmol) and naphthalen-2-ylboronic acid (4.7 g, 27.4 mmol) were added to 300 ml of THF, and the mixture was stirred and refluxed. Then, potassium carbonate (10.8 g, 78.4 mmol) was dissolved in 32 ml of water and added thereto, and the mixture was sufficiently stirred, and then bis(tri-tert-butylphosphine)palladium(0) (0.1 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.6 g of Compound 1-92. (Yield: 67%, MS: [M+H]+=666)

### Preparation Example 1-93

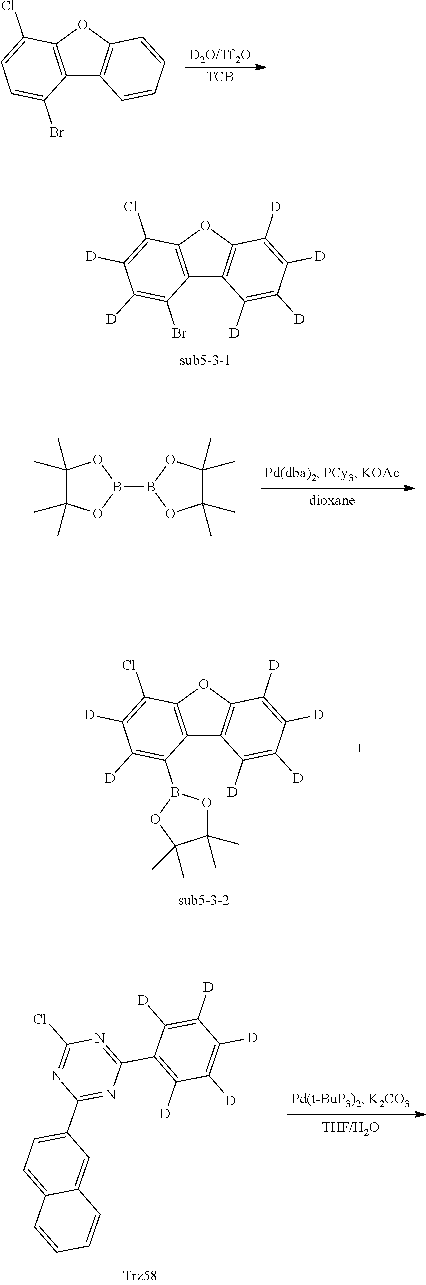

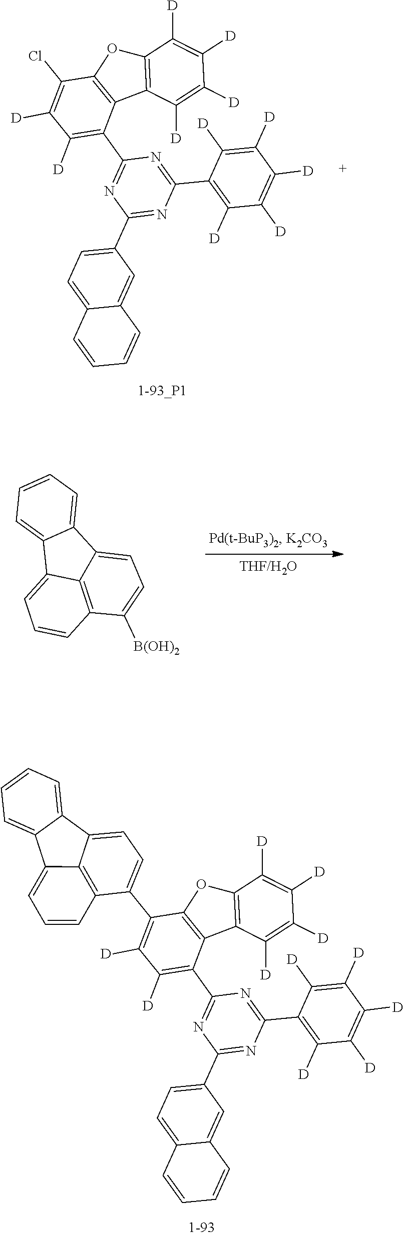 [SMILES: [2H]c1c([2H])c([2H])c(-c2nc(-c3ccc4ccccc4c3)nc(-c3c([2H])c([2H])c(Cl)c4oc5c([2H])c([2H])c([2H])c([2H])c5c34)n2)c([2H])c1[2H] ; OB(O)c1ccc2c3c(cccc13)-c1ccccc1-2 ; [2H]c1c([2H])c([2H])c(-c2nc(-c3ccc4ccccc4c3)nc(-c3c([2H])c([2H])c(-c4ccc5c6c(cccc46)-c4ccccc4-5)c4oc5c([2H])c([2H])c([2H])c([2H])c5c34)n2)c([2H])c1[2H]]

Trifluoromethanesulfonic anhydride (90.2 g, 319.7 mmol) and deuterium oxide (32 g, 1598.4 mmol) were added at 0° C. and stirred for 5 hours to prepare a solution. 1-bromo-4-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-4-chlorodibenzo[b,d]furan and 1,2,4-trichlorobenzene, and the mixture was stirred while heating to 140° C. and then keeping that temperature. After the reaction for 18 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.8 g of sub5-3-1. (Yield: 38%, MS: [M+H]+=287)

Sub5-3-1 (15 g, 52.2 mmol) and bis(pinacolato)diboron (14.6 g, 57.4 mmol) were added to 300 ml of 1,4-dioxane, and the mixture was stirred and refluxed. Then, potassium acetate (7.7 g, 78.2 mmol) was added thereto, and the mixture was sufficiently stirred, and then bis(dibenzylideneacetone)palladium(0) (0.9 g, 1.6 mmol) and tricyclohexylphosphine (0.9 g, 3.1 mmol) were added. After the reaction for 6 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.9 g of sub5-3-2. (Yield: 74%, MS: [M+H]+=335)

Sub5-3-2 (15 g, 44.8 mmol) and Trz58 (15.7 g, 47.1 mmol) were added to 300 ml of THF, and the mixture was stirred and refluxed. Then, potassium carbonate (18.6 g, 134.5 mmol) was dissolved in 56 ml of water and added thereto, and the mixture was sufficiently stirred, and then bis(tri-tert-butylphosphine)palladium(0) (0.2 g, 0.4 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 16.2 g of Compound 1-93_P1. (Yield: 73%, MS: [M+H]+=495)

Compound 1-93_P1 (15 g, 30.3 mmol) and fluoranthen-3-ylboronic acid (7.8 g, 31.8 mmol) were added to 300 ml of THF, and the mixture was stirred and refluxed. Then, potassium carbonate (12.6 g, 90.9 mmol) was dissolved in 38 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 15 g of Compound 1-93. (Yield: 75%, MS: [M+H]+=661)

### Preparation Example 1-94

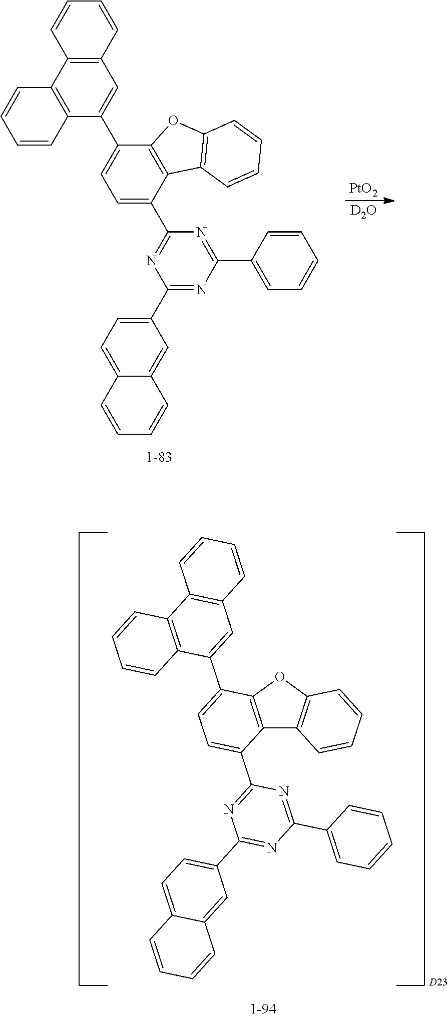

Compound 1-83 (10 g, 16 mmol), PtO₂ (1.1 g, 4.8 mmol) and D₂O (80 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 3.9 g of Compound 1-94. (Yield: 38%, MS: [M+H]+=650)

### Preparation Example 1-95

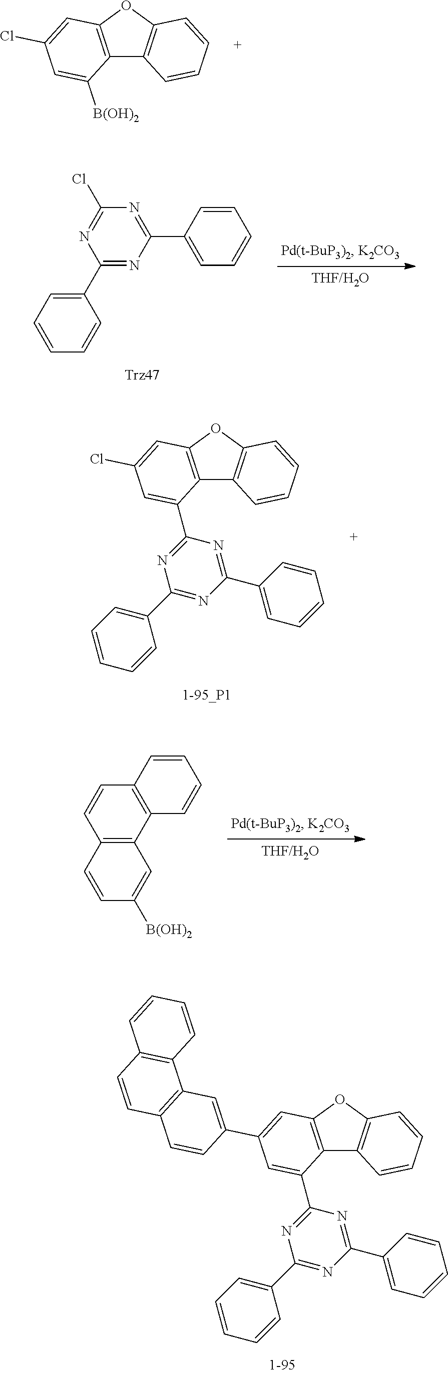 [SMILES: OB(O)c1cc(Cl)cc2oc3ccccc3c12 ; Clc1nc(-c2ccccc2)nc(-c2ccccc2)n1 ; Clc1cc(-c2nc(-c3ccccc3)nc(-c3ccccc3)n2)c2c(c1)oc1ccccc12 ; OB(O)c1ccc2ccc3ccccc3c2c1 ; c1ccc(-c2nc(-c3ccccc3)nc(-c3cc(-c4ccc5ccc6ccccc6c5c4)cc4oc5ccccc5c34)n2)cc1]

(3-Chlorodibenzo[b,d]furan-1-yl)boronic acid (15 g, 60.9 mmol) and Trz47 (17.1 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 19 g of Compound 1-95_P1. (Yield: 72%, MS: [M+H]+=434)

Compound 1-95_P1 (15 g, 34.6 mmol) and phenanthren-3-ylboronic acid (8.1 g, 36.3 mmol) were added to 300 ml of THF, and the mixture was stirred and refluxed. Then, potassium carbonate (14.3 g, 103.7 mmol) was dissolved in 43 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 14.1 g of Compound 1-95. (Yield: 71%, MS: [M+H]+=576)

### Preparation Example 1-96

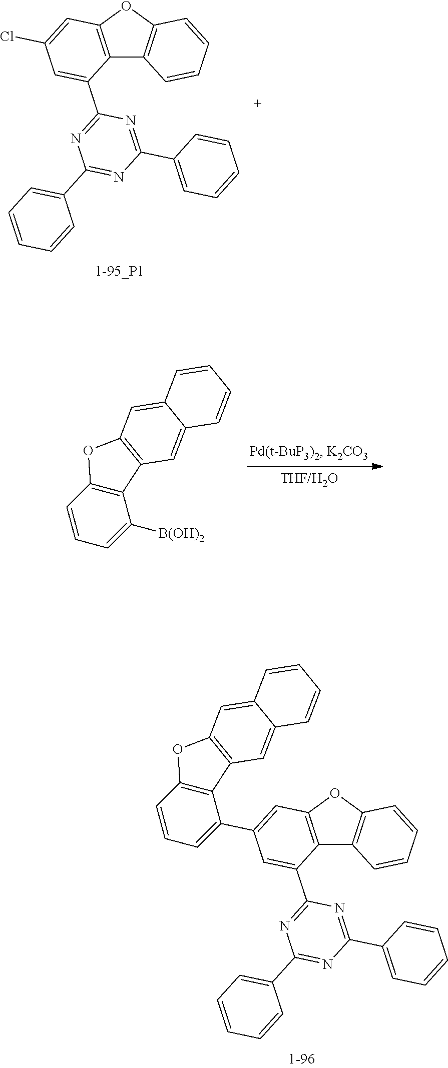 [SMILES: Clc1cc(-c2nc(-c3ccccc3)nc(-c3ccccc3)n2)c2c(c1)oc1ccccc12 ; OB(O)c1cccc2oc3cc4ccccc4cc3c12 ; c1ccc(-c2nc(-c3ccccc3)nc(-c3cc(-c4cccc5oc6cc7ccccc7cc6c45)cc4oc5ccccc5c34)n2)cc1]

Compound 1-96_P1 (15 g, 34.6 mmol) and naphtho[2,3-b]benzofuran-1-ylboronic acid (9.5 g, 36.3 mmol) were added to 300 ml of THF, and the mixture was stirred and refluxed. Then, potassium carbonate (14.3 g, 103.7 mmol) was dissolved in 43 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 16 g of Compound 1-96. (Yield: 75%, MS: [M+H]+=616)

### Preparation Example 1-97

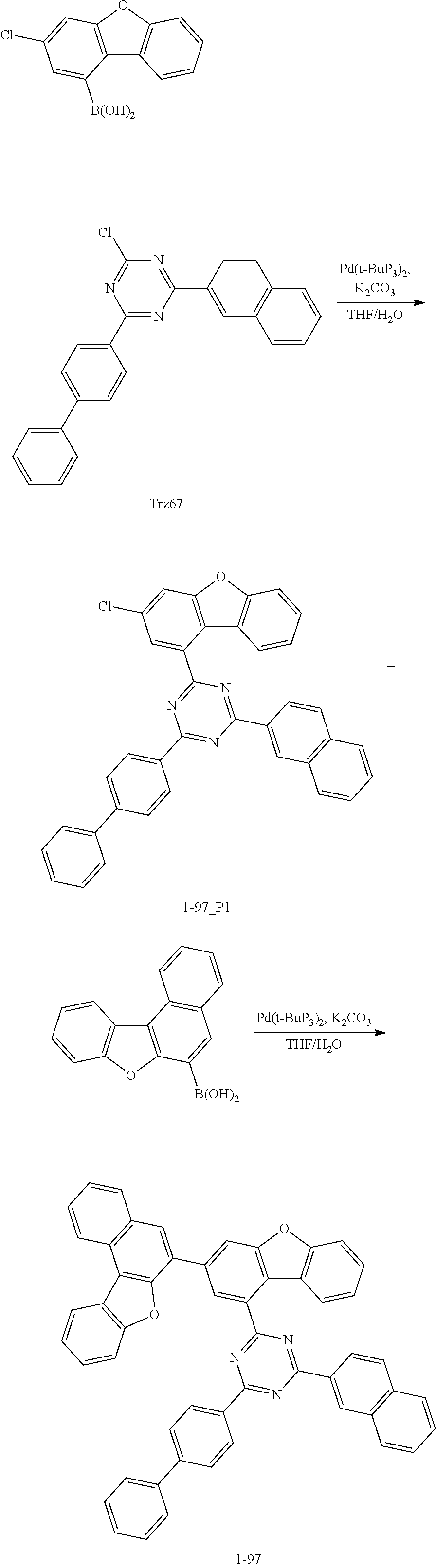 [SMILES: OB(O)c1cc(Cl)cc2oc3ccccc3c12 ; Clc1nc(-c2ccc(-c3ccccc3)cc2)nc(-c2ccc3ccccc3c2)n1 ; Clc1cc(-c2nc(-c3ccc(-c4ccccc4)cc3)nc(-c3ccc4ccccc4c3)n2)c2c(c1)oc1ccccc12 ; OB(O)c1cc2ccccc2c2c1oc1ccccc12 ; c1ccc(-c2ccc(-c3nc(-c4ccc5ccccc5c4)nc(-c4cc(-c5cc6ccccc6c6c5oc5ccccc56)cc5oc6ccccc6c45)n3)cc2)cc1]

(3-chlorodibenzo[b,d]furan-1-yl)boronic acid (15 g, 60.9 mmol) and Trz67 (25.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 25.2 g of Compound 1-97_P1. (Yield: 74%, MS: [M+H]+=560)

Compound 1-97_P1 (15 g, 26.8 mmol) and naphtho[2,1-b]benzofuran-6-ylboronic acid (7.4 g, 28.1 mmol) were added to 300 ml of THF, and the mixture was stirred and refluxed. Then, potassium carbonate (11.1 g, 80.3 mmol) was dissolved in 33 ml of water and added thereto, and the mixture was sufficiently stirred, and then bis(tri-tert-butylphosphine)palladium(0) (0.1 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 14.5 g of Compound 1-97. (Yield: 73%, MS: [M+H]+=742)

### Preparation Example 1-98

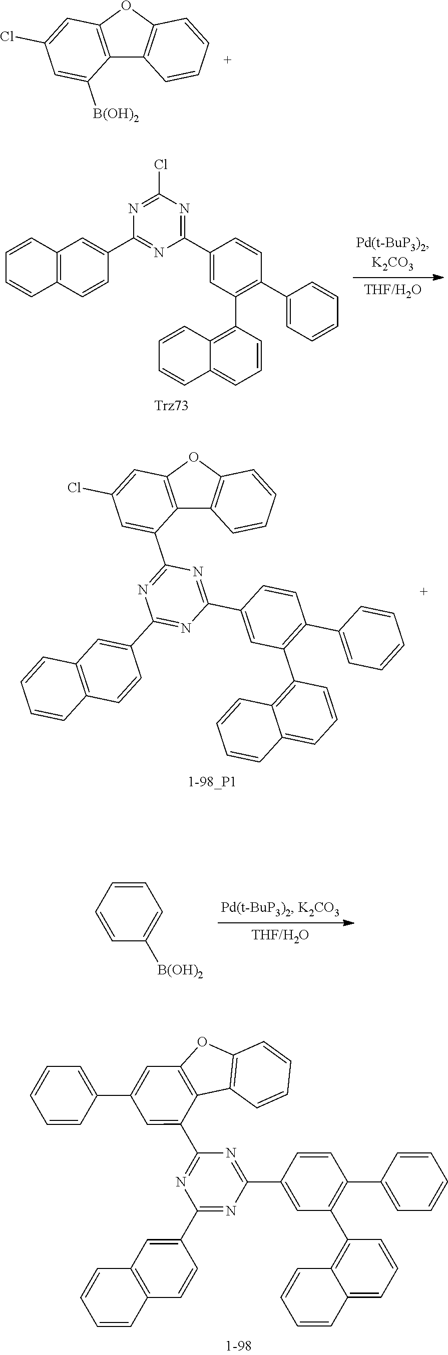 [SMILES: OB(O)c1cc(Cl)cc2oc3ccccc3c12 ; Clc1nc(-c2ccc(-c3ccccc3)c(-c3cccc4ccccc34)c2)nc(-c2ccc3ccccc3c2)n1 ; Clc1cc(-c2nc(-c3ccc(-c4ccccc4)c(-c4cccc5ccccc45)c3)nc(-c3ccc4ccccc4c3)n2)c2c(c1)oc1ccccc12 ; OB(O)c1ccccc1 ; c1ccc(-c2cc(-c3nc(-c4ccc(-c5ccccc5)c(-c5cccc6ccccc56)c4)nc(-c4ccc5ccccc5c4)n3)c3c(c2)oc2ccccc23)cc1]

(3-Chlorodibenzo[b,d]furan-1-yl)boronic acid (15 g, 60.9 mmol) and Trz73 (33.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 29.6 g of Compound 1-98_P1. (Yield: 71%, MS: [M+H]+=686)

Compound 1-98_P1 (15 g, 21.9 mmol) and phenylboronic acid (2.8 g, 23 mmol) were added to 300 ml of THF, and the mixture was stirred and refluxed. Then, potassium carbonate (9.1 g, 65.6 mmol) was dissolved in 27 ml of water and added thereto, and the mixture was sufficiently stirred, and then bis(tri-tert-butylphosphine)palladium(0) (0.1 g, 0.2 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.3 g of Compound 1-98. (Yield: 68%, MS: [M+H]+=758)

### Preparation Example 1-99

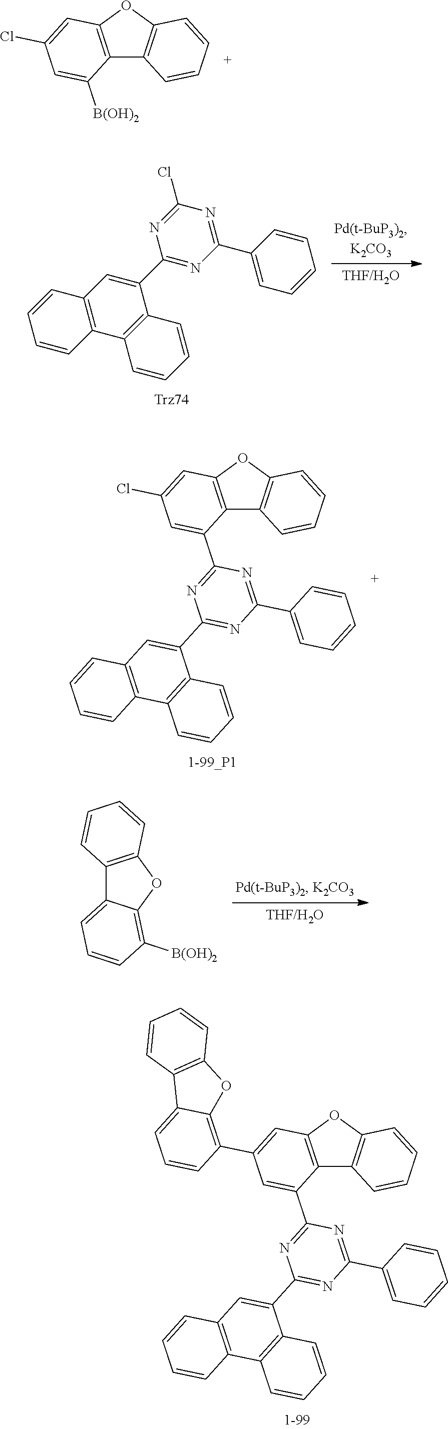 [SMILES: OB(O)c1cc(Cl)cc2oc3ccccc3c12 ; Clc1nc(-c2ccccc2)nc(-c2cc3ccccc3c3ccccc23)n1 ; Clc1cc(-c2nc(-c3ccccc3)nc(-c3cc4ccccc4c4ccccc34)n2)c2c(c1)oc1ccccc12 ; OB(O)c1cccc2c1oc1ccccc12 ; c1ccc(-c2nc(-c3cc4ccccc4c4ccccc34)nc(-c3cc(-c4cccc5c4oc4ccccc45)cc4oc5ccccc5c34)n2)cc1]

(3-Chlorodibenzo[b,d]furan-1-yl)boronic acid (15 g, 60.9 mmol) and Trz74 (23.5 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 22.7 g of Compound 1-99_P1. (Yield: 70%, MS: [M+H]+=534)

Compound 1-99_P1 (15 g, 28.1 mmol) and dibenzo[b,d]furan-4-ylboronic acid (6.3 g, 29.5 mmol) were added to 300 ml of THF, and the mixture was stirred and refluxed. Then, potassium carbonate (11.6 g, 84.3 mmol) was dissolved in 35 ml of water and added thereto, and the mixture was sufficiently stirred, and then bis(tri-tert-butylphosphine)palladium(0) (0.1 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.9 g of Compound 1-99. (Yield: 69%, MS: [M+H]+=666)

### Preparation Example 1-100

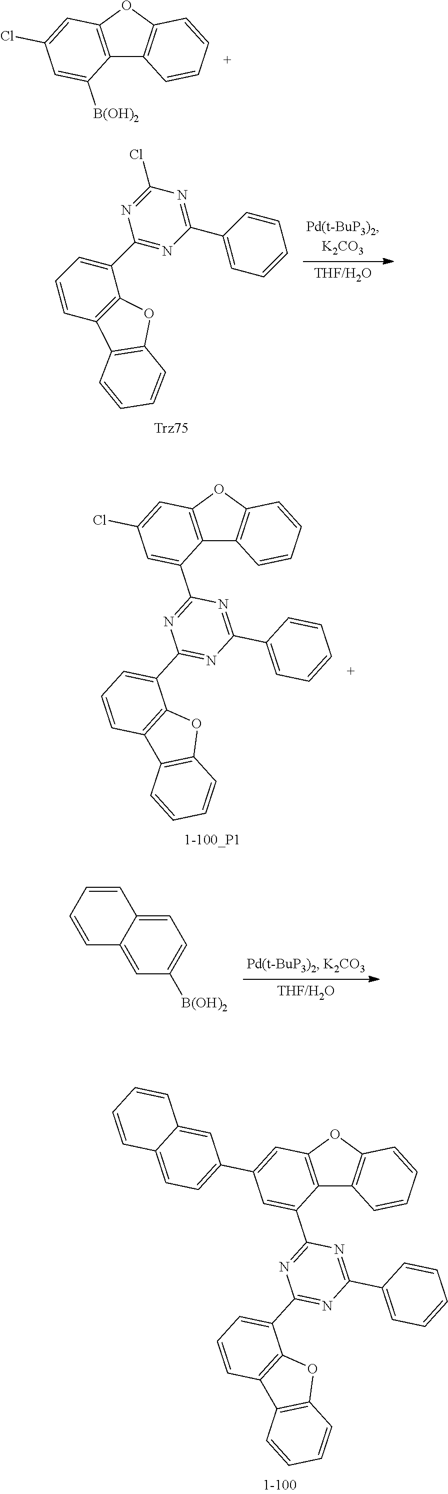 [SMILES: OB(O)c1cc(Cl)cc2oc3ccccc3c12 ; Clc1nc(-c2ccccc2)nc(-c2cccc3c2oc2ccccc23)n1 ; Clc1cc(-c2nc(-c3ccccc3)nc(-c3cccc4c3oc3ccccc34)n2)c2c(c1)oc1ccccc12 ; OB(O)c1ccc2ccccc2c1 ; c1ccc(-c2nc(-c3cccc4c3oc3ccccc34)nc(-c3cc(-c4ccc5ccccc5c4)cc4oc5ccccc5c34)n2)cc1]

(3-Chlorodibenzo[b,d]furan-1-yl)boronic acid (15 g, 60.9 mmol) and Trz75 (22.9 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 21.3 g of Compound 1-100_P1. (Yield: 67%, MS: [M+H]+=524)

Compound 1-100_P1 (15 g, 28.6 mmol) and naphthalen-2-ylboronic acid (5.2 g, 30.1 mmol) were added to 300 ml of THF, and the mixture was stirred and refluxed. Then, potassium carbonate (11.9 g, 85.9 mmol) was dissolved in 36 ml of water and added thereto, and the mixture was sufficiently stirred, and then bis(tri-tert-butylphosphine)palladium(0) (0.1 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.7 g of Compound 1-100. (Yield: 72%, MS: [M+H]+=616)

### Preparation Example 1-101

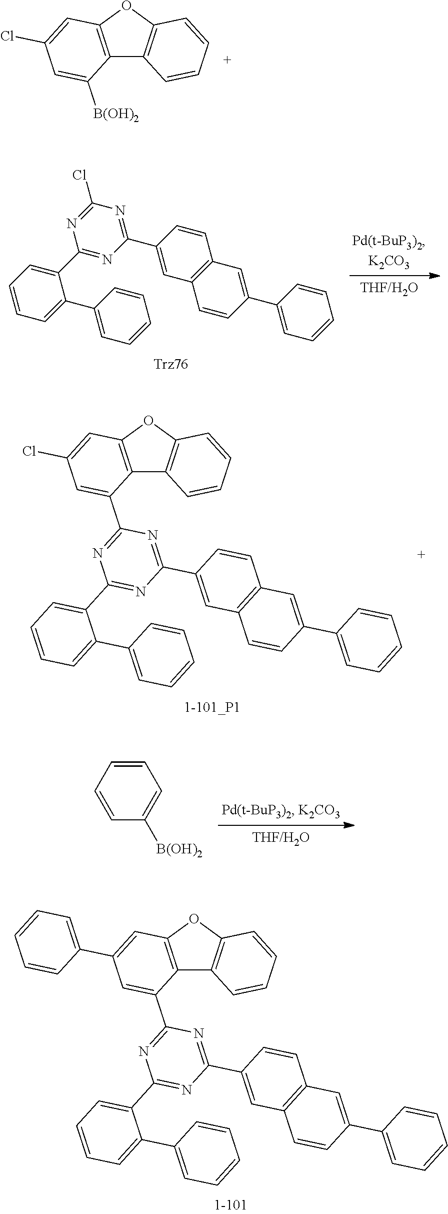 [SMILES: OB(O)c1cc(Cl)cc2oc3ccccc3c12 ; Clc1nc(-c2ccc3cc(-c4ccccc4)ccc3c2)nc(-c2ccccc2-c2ccccc2)n1 ; Clc1cc(-c2nc(-c3ccc4cc(-c5ccccc5)ccc4c3)nc(-c3ccccc3-c3ccccc3)n2)c2c(c1)oc1ccccc12 ; OB(O)c1ccccc1 ; c1ccc(-c2ccc3cc(-c4nc(-c5ccccc5-c5ccccc5)nc(-c5cc(-c6ccccc6)cc6oc7ccccc7c56)n4)ccc3c2)cc1]

(3-Chlorodibenzo[b,d]furan-1-yl)boronic acid (15 g, 60.9 mmol) and Trz76 (30 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 25.5 g of Compound 1-101_P1. (Yield: 66%, MS: [M+H]+=636)

Compound 1-101_P1 (15 g, 23.6 mmol) and phenylboronic acid (3 g, 24.8 mmol) were added to 300 ml of THF, and the mixture was stirred and refluxed. Then, potassium carbonate (9.8 g, 70.7 mmol) was dissolved in 29 ml of water and added thereto, and the mixture was sufficiently stirred, and then bis(tri-tert-butylphosphine)palladium(0) (0.1 g, 0.2 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 10.7 g of Compound 1-101. (Yield: 67%, MS: [M+H]+=678)

### Preparation Example 1-102

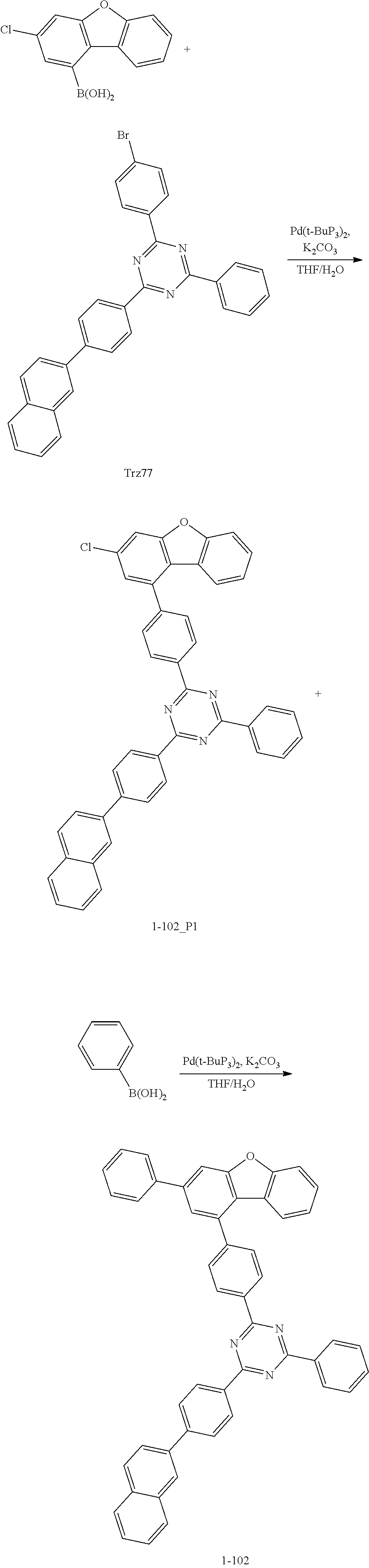 [SMILES: OB(O)c1cc(Cl)cc2oc3ccccc3c12 ; Brc1ccc(-c2nc(-c3ccccc3)nc(-c3ccc(-c4ccc5ccccc5c4)cc3)n2)cc1 ; Clc1cc(-c2ccc(-c3nc(-c4ccccc4)nc(-c4ccc(-c5ccc6ccccc6c5)cc4)n3)cc2)c2c(c1)oc1ccccc12 ; OB(O)c1ccccc1 ; c1ccc(-c2cc(-c3ccc(-c4nc(-c5ccccc5)nc(-c5ccc(-c6ccc7ccccc7c6)cc5)n4)cc3)c3c(c2)oc2ccccc23)cc1]

(3-Chlorodibenzo[b,d]furan-1-yl)boronic acid (15 g, 60.9 mmol) and Trz77 (32.9 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 26.3 g of Compound 1-102_P1. (Yield: 68%, MS: [M+H]+=636)

Compound 1-102_P1 (15 g, 23.6 mmol) and phenylboronic acid (3 g, 24.8 mmol) were added to 300 ml of THF, and the mixture was stirred and refluxed. Then, potassium carbonate (9.8 g, 70.7 mmol) was dissolved in 29 ml of water and added thereto, and the mixture was sufficiently stirred, and then bis(tri-tert-butylphosphine)palladium(0) (0.1 g, 0.2 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 10.5 g of Compound 1-102. (Yield: 66%, MS: [M+H]+=678)

### Preparation Example 1-103

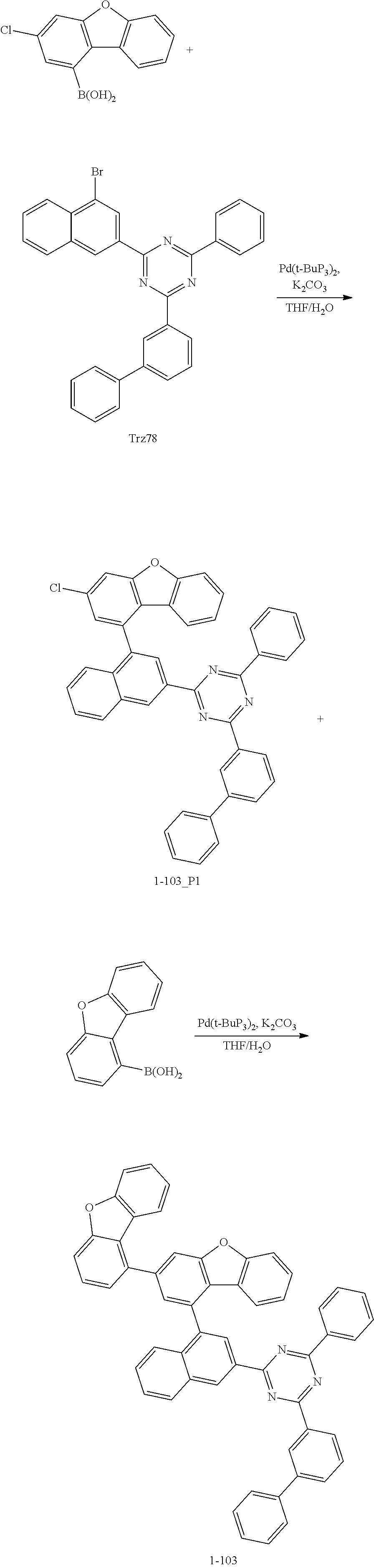 [SMILES: OB(O)c1cc(Cl)cc2oc3ccccc3c12 ; Brc1cc(-c2nc(-c3ccccc3)nc(-c3cccc(-c4ccccc4)c3)n2)cc2ccccc12 ; Clc1cc(-c2cc(-c3nc(-c4ccccc4)nc(-c4cccc(-c5ccccc5)c4)n3)cc3ccccc23)c2c(c1)oc1ccccc12 ; OB(O)c1cccc2oc3ccccc3c12 ; c1ccc(-c2cccc(-c3nc(-c4ccccc4)nc(-c4cc(-c5cc(-c6cccc7oc8ccccc8c67)cc6oc7ccccc7c56)c5ccccc5c4)n3)c2)cc1]

(3-Chlorodibenzo[b,d]furan-1-yl)boronic acid (15 g, 60.9 mmol) and Trz78 (32.9 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 25.9 g of Compound 1-103_P1. (Yield: 67%, MS: [M+H]+=636)

Compound 1-103_P1 (15 g, 23.6 mmol) and dibenzo[b,d]furan-1-ylboronic acid (5.2 g, 24.8 mmol) were added to 300 ml of THF, and the mixture was stirred and refluxed. Then, potassium carbonate (9.8 g, 70.7 mmol) was dissolved in 29 ml of water and added thereto, and the mixture was sufficiently stirred, and then bis(tri-tert-butylphosphine)palladium(0) (0.1 g, 0.2 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.1 g of Compound 1-103. (Yield: 67%, MS: [M+H]+=768)

### Preparation Example 1-104

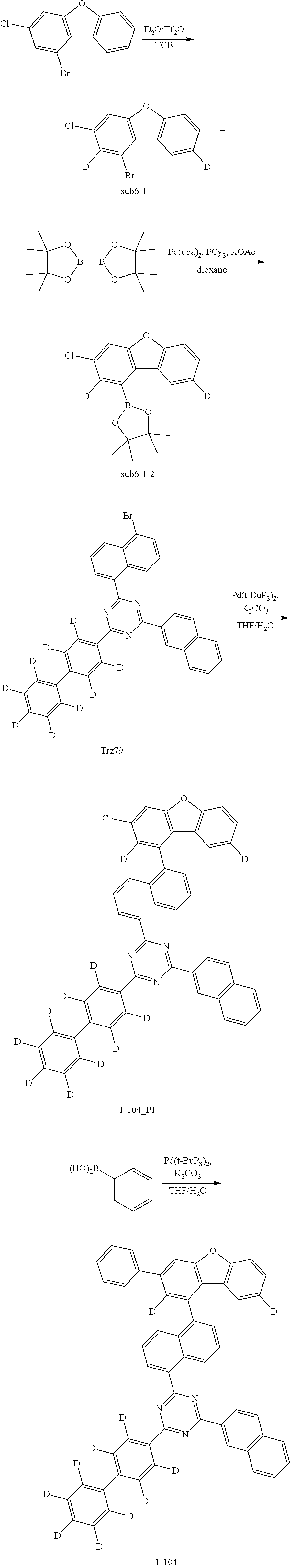

Trifluoromethanesulfonic anhydride (30.1 g, 106.6 mmol) and deuterium oxide (10.7 g, 532.8 mmol) were added at 0° C. and stirred for 5 hours to prepare a solution. 1-Bromo-3-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-3-chlorodibenzo[b,d]furan and 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6 g of sub6-1-1. (Yield: 40%, MS: [M+H]+=283)

Sub6-1-1 (15 g, 52.9 mmol) and bis(pinacolato)diboron (14.8 g, 58.2 mmol) were added to 300 ml of 1,4-dioxane, and the mixture was stirred and refluxed. Then, potassium acetate (7.8 g, 79.4 mmol) was added thereto, and the mixture was sufficiently stirred, and then bis(dibenzylideneacetone)palladium(0) (0.9 g, 1.6 mmol) and tricyclohexylphosphine (0.9 g, 3.2 mmol) were added. After the reaction for 6 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 9.8 g of sub6-1-2. (Yield: 56%, MS: [M+H]+=331)

Sub6-1-2 (15 g, 45.4 mmol) and Trz79 (27.3 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred, and then bis(tri-tert-butylphosphine)palladium(0) (0.2 g, 0.5 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 21.8 g of Compound 1-104_P1. (Yield: 69%, MS: [M+H]+=698)

Compound 1-104_P1 (15 g, 21.5 mmol) and phenylboronic acid (2.8 g, 22.6 mmol) were added to 300 ml of THF, and the mixture was stirred and refluxed. Then, potassium carbonate (8.9 g, 64.5 mmol) was dissolved in 27 ml of water and added thereto, and the mixture was sufficiently stirred, and then bis(tri-tert-butylphosphine)palladium(0) (0.1 g, 0.2 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.6 g of Compound 1-104. (Yield: 73%, MS: [M+H]+=739)

### Preparation Example 1-105

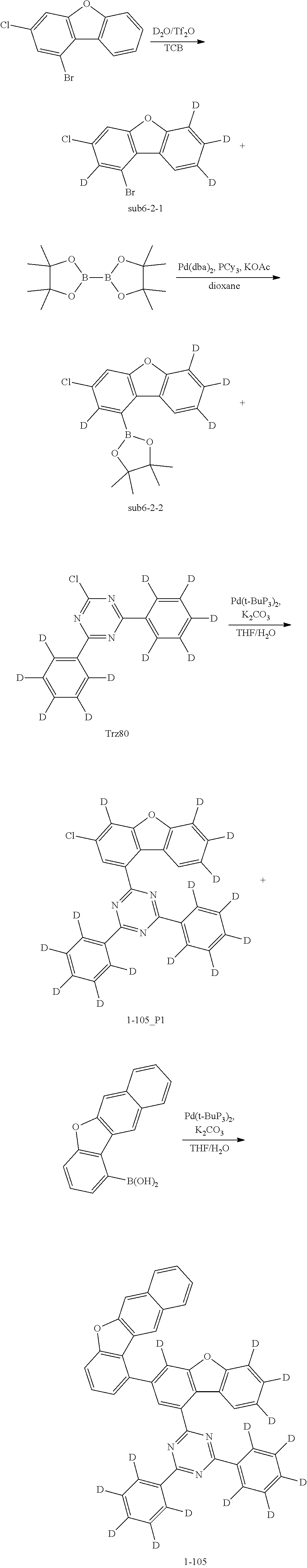

Trifluoromethanesulfonic anhydride (60.1 g, 213.1 mmol) and deuterium oxide (21.4 g, 1065.6 mmol) were added at 0° C. and stirred for 5 hours to prepare a solution. 1-Bromo-3-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-3-chlorodibenzo[b,d]furan and 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.2 g of sub6-2-1. (Yield: 41%, MS: [M+H]+=285)

Sub6-2-1 (15 g, 52.9 mmol) and bis(pinacolato)diboron (14.8 g, 58.2 mmol) were added to 300 ml of 1,4-dioxane, and the mixture was stirred and refluxed. Then, potassium acetate (7.8 g, 79.4 mmol) was added thereto, and the mixture was sufficiently stirred, and then bis(dibenzylideneacetone)palladium(0) (0.9 g, 1.6 mmol) and tricyclohexylphosphine (0.9 g, 3.2 mmol) were added. After the reaction for 6 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 10.8 g of sub6-2-2. (Yield: 62%, MS: [M+H]+=331)

Sub6-2-2 (15 g, 45.1 mmol) and Trz80 (13.2 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred, and then bis(tri-tert-butylphosphine)palladium(0) (0.2 g, 0.5 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.1 g of Compound 1-105_P1. (Yield: 65%, MS: [M+H]+=448)

Compound 1-105_P1 (15 g, 33.5 mmol) and naphtho[2,3-b]benzofuran-1-ylboronic acid (9.2 g, 35.2 mmol) were added to 300 ml of THF, and the mixture was stirred and refluxed. Then, potassium carbonate (13.9 g, 100.5 mmol) was dissolved in 42 ml of water and added thereto, and the mixture was sufficiently stirred, and then bis(tri-tert-butylphosphine)palladium(0) (0.2 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 15.6 g of Compound 1-105. (Yield: 74%, MS: [M+H]+=630)

### Preparation Example 1-106

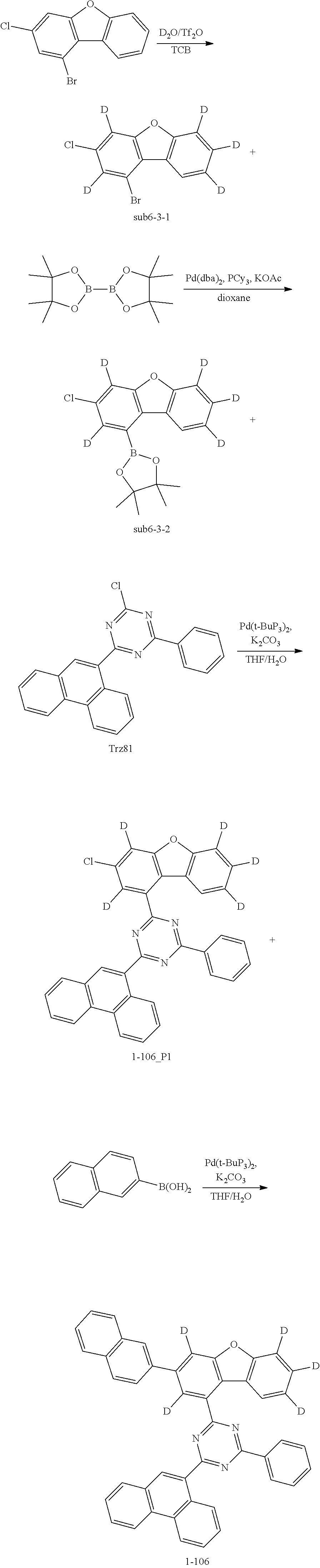

Trifluoromethanesulfonic anhydride (75.2 g, 266.4 mmol) and deuterium oxide (26.7 g, 1332 mmol) were added at 0° C. and stirred for 5 hours to prepare a solution. 1-bromo-3-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-3-chlorodibenzo[b,d]furan and 1,2,4-trichlorobenzene, and the mixture was stirred while heating to 140° C. and then keeping that temperature. After the reaction for 12 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.6 g of sub6-3-1. (Yield: 37%, MS: [M+H]+=286)

Sub6-3-1 (15 g, 52.3 mmol) and bis(pinacolato)diboron (14.6 g, 57.6 mmol) were added to 300 ml of 1,4-dioxane, and the mixture was stirred and refluxed. Then, potassium acetate (7.7 g, 78.5 mmol) was added thereto, and the mixture was sufficiently stirred, and then bis(dibenzylideneacetone)palladium(0) (0.9 g, 1.6 mmol) and tricyclohexylphosphine (0.9 g, 3.1 mmol) were added. After the reaction for 6 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12 g of sub6-3-2. (Yield: 69%, MS: [M+H]+=334)

Sub6-3-2 (15 g, 45 mmol) and Trz81 (17.4 g, 47.2 mmol) were added to 300 ml of THF, and the mixture was stirred and refluxed. Then, potassium carbonate (18.6 g, 134.9 mmol) was dissolved in 56 ml of water and added thereto, and the mixture was sufficiently stirred, and then bis(tri-tert-butylphosphine)palladium(0) (0.2 g, 0.4 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 17.2 g of Compound 1-106_P1. (Yield: 71%, MS: [M+H]+=539.

Compound 1-106_P1 (15 g, 27.8 mmol) and naphthalen-2-ylboronic acid (5 g, 29.2 mmol) were added to 300 ml of THF, and the mixture was stirred and refluxed. Then, potassium carbonate (11.5 g, 83.5 mmol) was dissolved in 35 ml of water and added thereto, and the mixture was sufficiently stirred, and then bis(tri-tert-butylphosphine)palladium(0) (0.1 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.3 g of Compound 1-106. (Yield: 70%, MS: [M+H]+=631)

### Preparation Example 1-107

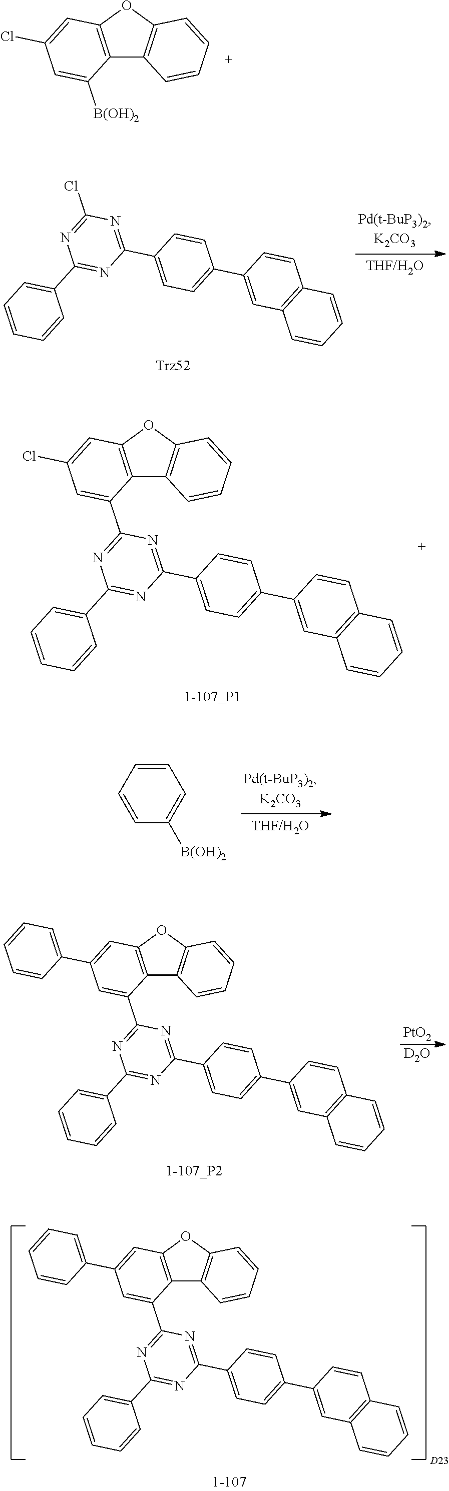

(3-Chlorodibenzo[b,d]furan-1-yl)boronic acid (15 g, 60.9 mmol) and Trz52 (25.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 22.8 g of Compound 1-107_P1. (Yield: 67%, MS: [M+H]+=560)

Compound 1-107_P1 (15 g, 26.8 mmol) and phenylboronic acid (3.4 g, 28.1 mmol) were added to 300 ml of THF, and the mixture was stirred and refluxed. Then, potassium carbonate (11.1 g, 80.3 mmol) was dissolved in 33 ml of water and added thereto, and the mixture was sufficiently stirred, and then bis(tri-tert-butylphosphine)palladium(0) (0.1 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 10.9 g of Compound 1-107_P2. (Yield: 68%, MS: [M+H]+=602)

Compound 1-107_P2 (10 g, 16.6 mmol), PtO₂ (1.1 g, 5 mmol) and D₂O (83 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 4 g of Compound 1-107. (Yield: 39%, MS: [M+H]+=626)

### Preparation Example 1-108

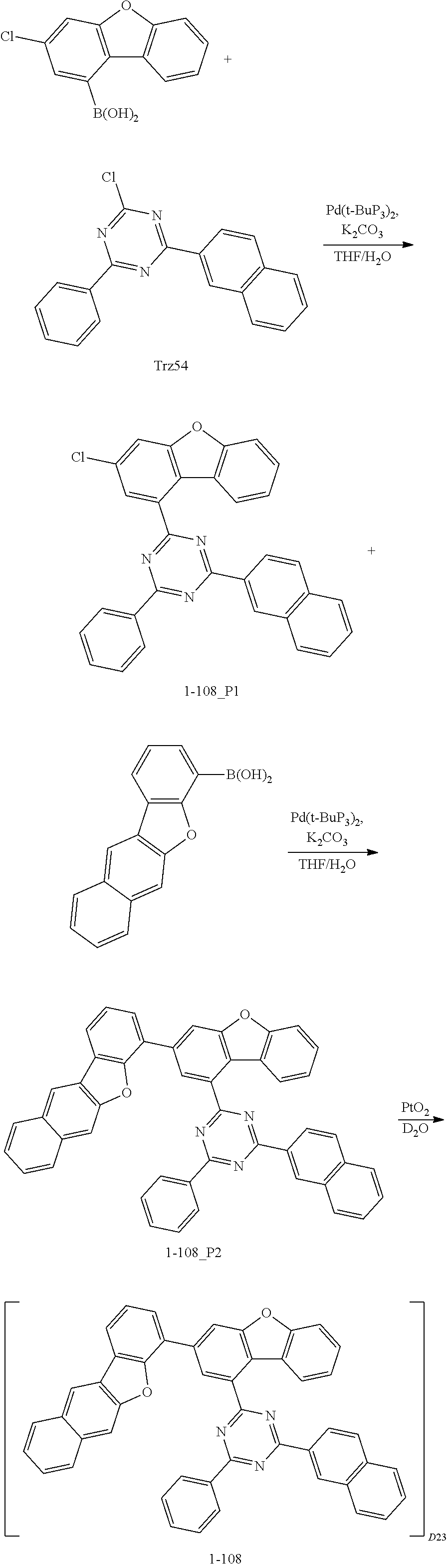

(3-Chlorodibenzo[b,d]furan-1-yl)boronic acid (15 g, 60.9 mmol) and Trz54 (20.3 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 21.2 g of Compound 1-108_P1. (Yield: 72%, MS: [M+H]+=484)

Compound 1-108_P1 (15 g, 31 mmol) and naphtho[2,3-b]benzofuran-4-ylboronic acid (8.5 g, 32.5 mmol) were added to 300 ml of THF, and the mixture was stirred and refluxed. Then, potassium carbonate (12.9 g, 93 mmol) was dissolved in 39 ml of water and added thereto, and the mixture was sufficiently stirred, and then bis(tri-tert-butylphosphine)palladium(0) (0.2 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 14.2 g of Compound 1-108_P2. (Yield: 69%, MS: [M+H]+=666)

Compound 1-108_P2 (10 g, 15 mmol), PtO₂ (1 g, 4.5 mmol) and D₂O (75 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 3.8 g of Compound 1-108. (Yield: 37%, MS: [M+H]+=690)

### Preparation Example 1-109

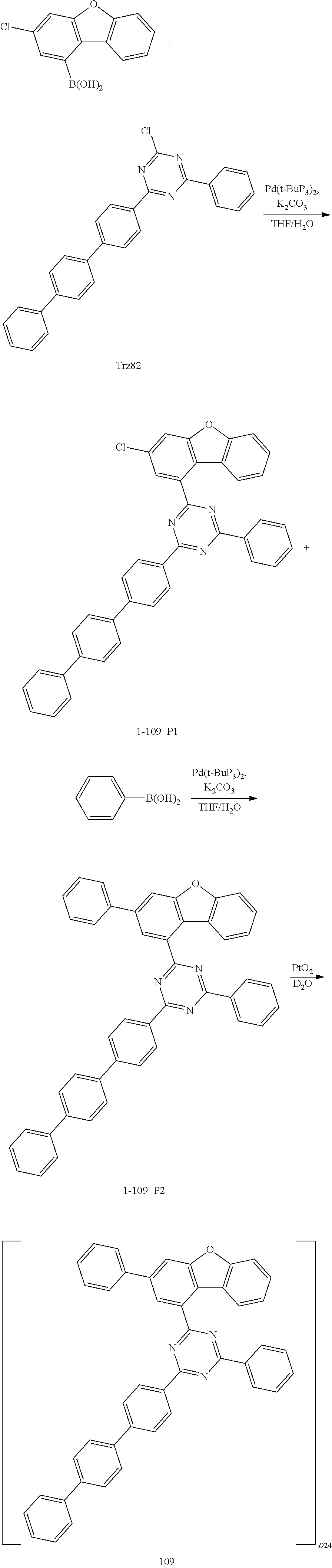

(3-Chlorodibenzo[b,d]furan-1-yl)boronic acid (15 g, 60.9 mmol) and Trz82 (26.8 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 25.3 g of Compound 1-109_P1. (Yield: 71%, MS: [M+H]+=586)

Compound 1-109_P1 (15 g, 25.6 mmol) and phenylboronic acid (3.3 g, 26.9 mmol) were added to 300 ml of THF, and the mixture was stirred and refluxed. Then, potassium carbonate (10.6 g, 76.8 mmol) was dissolved in 32 ml of water and added thereto, and the mixture was sufficiently stirred, and then bis(tri-tert-butylphosphine)palladium(0) (0.1 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12 g of Compound 1-109_P2. (Yield: 75%, MS: [M+H]+=628)

Compound 1-109_P2 (10 g, 15.9 mmol), PtO₂ (1.1 g, 4.8 mmol) and D₂O (80 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 4 g of Compound 1-109. (Yield: 39%, MS: [M+H]+=653)

### Preparation Example 1-110

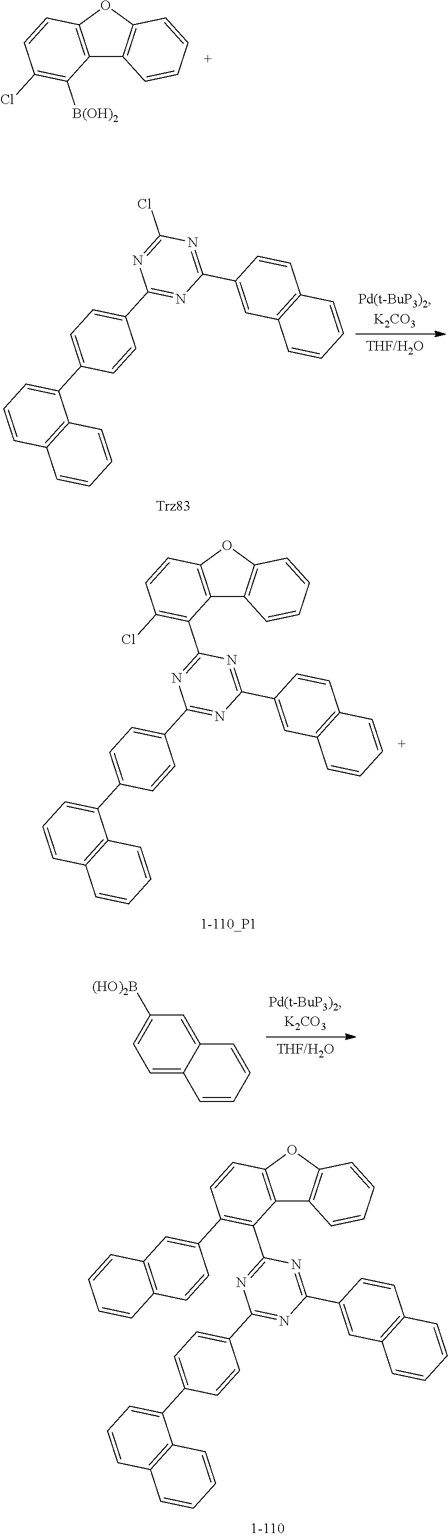 [SMILES: OB(O)c1c(Cl)ccc2oc3ccccc3c12 ; Clc1nc(-c2ccc(-c3cccc4ccccc34)cc2)nc(-c2ccc3ccccc3c2)n1 ; Clc1ccc2oc3ccccc3c2c1-c1nc(-c2ccc(-c3cccc4ccccc34)cc2)nc(-c2ccc3ccccc3c2)n1 ; OB(O)c1ccc2ccccc2c1 ; c1ccc2cc(-c3nc(-c4ccc(-c5cccc6ccccc56)cc4)nc(-c4c(-c5ccc6ccccc6c5)ccc5oc6ccccc6c45)n3)ccc2c1]

(2-Chlorodibenzo[b,d]furan-1-yl)boronic acid (15 g, 60.9 mmol) and Trz83 (28.4 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 24.8 g of Compound 1-110_P1. (Yield: 67%, MS: [M+H]+=610)

Compound 1-110_P1 (15 g, 24.6 mmol) and naphthalen-2-ylboronic acid (4.4 g, 25.8 mmol) were added to 300 ml of THF, and the mixture was stirred and refluxed. Then, potassium carbonate (10.2 g, 73.8 mmol) was dissolved in 31 ml of water and added thereto, and the mixture was sufficiently stirred, and then bis(tri-tert-butylphosphine)palladium(0) (0.1 g, 0.2 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.1 g of Compound 1-110. (Yield: 70%, MS: [M+H]+=702)

### Preparation Example 1-111

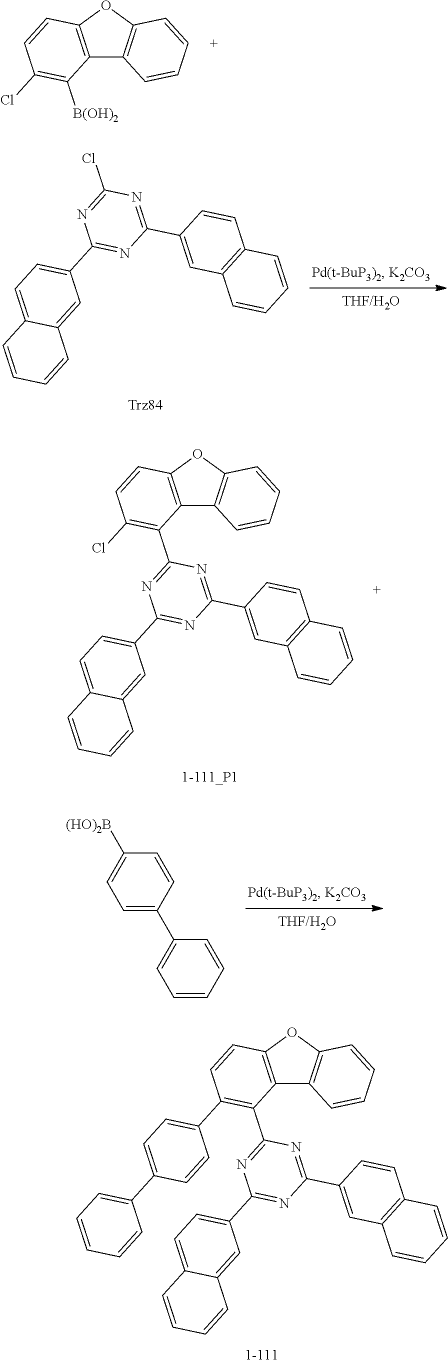 [SMILES: OB(O)c1c(Cl)ccc2oc3ccccc3c12 ; Clc1nc(-c2ccc3ccccc3c2)nc(-c2ccc3ccccc3c2)n1 ; Clc1ccc2oc3ccccc3c2c1-c1nc(-c2ccc3ccccc3c2)nc(-c2ccc3ccccc3c2)n1 ; OB(O)c1ccc(-c2ccccc2)cc1 ; c1ccc(-c2ccc(-c3ccc4oc5ccccc5c4c3-c3nc(-c4ccc5ccccc5c4)nc(-c4ccc5ccccc5c4)n3)cc2)cc1]

(2-Chlorodibenzo[b,d]furan-1-yl)boronic acid (15 g, 60.9 mmol) and Trz84 (23.5 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 23.4 g of Compound 1-111_P1. (Yield: 72%, MS: [M+H]+=534)

Compound 1-111_P1 (15 g, 28.1 mmol) and [1,1′-biphenyl]-4-ylboronic acid (5.8 g, 29.5 mmol) were added to 300 ml of THF, and the mixture was stirred and refluxed. Then, potassium carbonate (11.6 g, 84.3 mmol) was dissolved in 35 ml of water and added thereto, and the mixture was sufficiently stirred, and then bis(tri-tert-butylphosphine)palladium(0) (0.1 g, 0.3 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.1 g of Compound 1-111. (Yield: 66%, MS: [M+H]+=652)

### Preparation Example 1-112

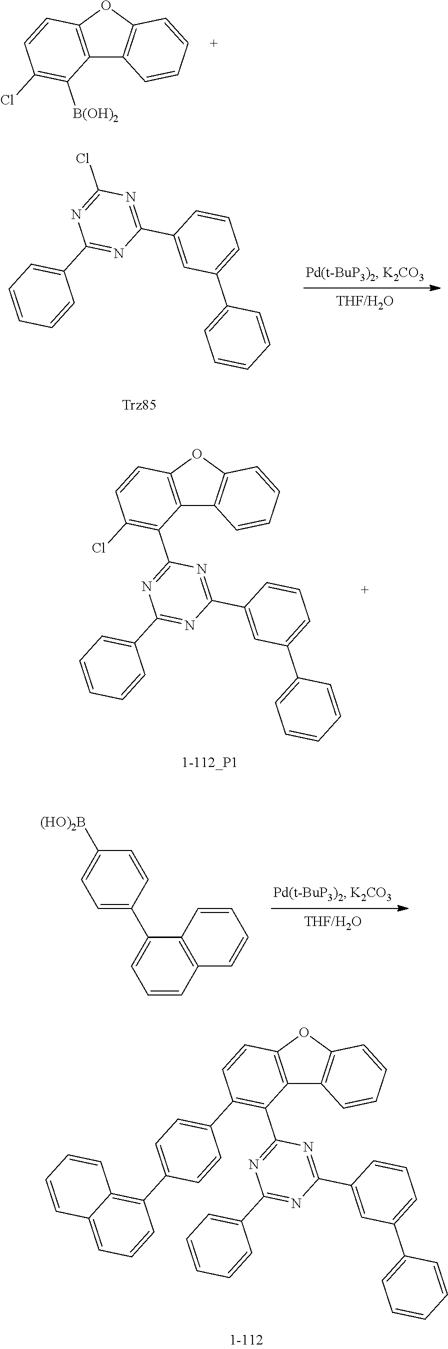

(2-Chlorodibenzo[b,d]furan-1-yl)boronic acid (15 g, 60.9 mmol) and Trz85 (22 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 20.1 g of Compound 1-112_P1. (Yield: 65%, MS: [M+H]+=510)

Compound 1-112_P1 (15 g, 29.4 mmol) and (4-(naphthalen-1-yl)phenyl)boronic acid (7.7 g, 30.9 mmol) were added to 300 ml of THF, and the mixture was stirred and refluxed. Then, potassium carbonate (12.2 g, 88.2 mmol) was dissolved in 37 ml of water and added thereto, and the mixture was sufficiently stirred, and then bis(tri-tert-butylphosphine)palladium(0) (0.2 g, 0.3 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 12.9 g of Compound 1-112. (Yield: 65%, MS: [M+H]+=678)

### Preparation Example 1-113

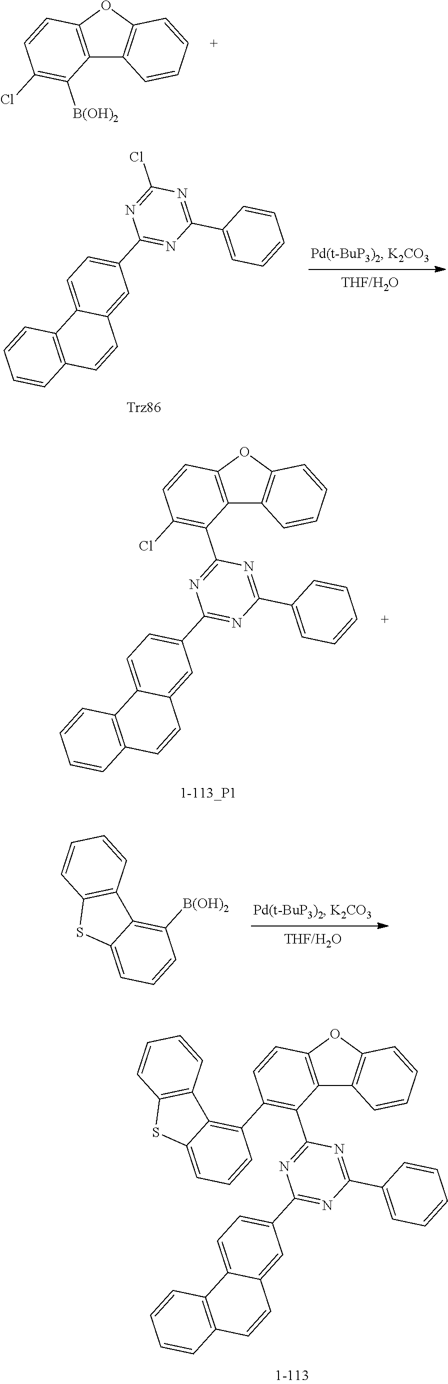 [SMILES: OB(O)c1c(Cl)ccc2oc3ccccc3c12 ; Clc1nc(-c2ccccc2)nc(-c2ccc3c(ccc4ccccc43)c2)n1 ; Clc1ccc2oc3ccccc3c2c1-c1nc(-c2ccccc2)nc(-c2ccc3c(ccc4ccccc43)c2)n1 ; OB(O)c1cccc2sc3ccccc3c12 ; c1ccc(-c2nc(-c3ccc4c(ccc5ccccc54)c3)nc(-c3c(-c4cccc5sc6ccccc6c45)ccc4oc5ccccc5c34)n2)cc1]

(2-Chlorodibenzo[b,d]furan-1-yl)boronic acid (15 g, 60.9 mmol) and Trz86 (23.5 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 23 g of Compound 1-113_P1. (Yield: 71%, MS: [M+H]+=534)

Compound 1-113_P1 (15 g, 28.1 mmol) and dibenzo[b,d]thiophen-1-ylboronic acid (6.7 g, 29.5 mmol) were added to 300 ml of THF, and the mixture was stirred and refluxed. Then, potassium carbonate (11.6 g, 84.3 mmol) was dissolved in 35 ml of water and added thereto, and the mixture was sufficiently stirred, and then bis(tri-tert-butylphosphine)palladium(0) (0.1 g, 0.3 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 13.4 g of Compound 1-113. (Yield: 70%, MS: [M+H]+=682)

### Preparation Example 1-114

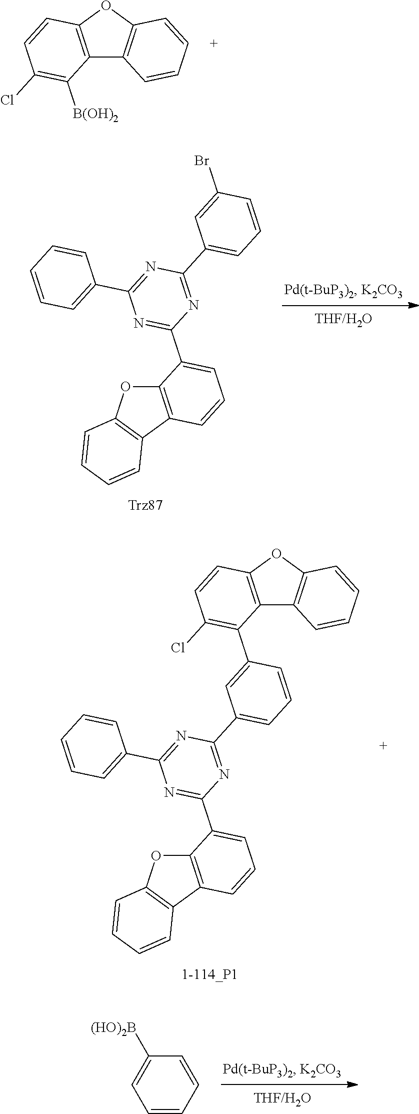 [SMILES: OB(O)c1c(Cl)ccc2oc3ccccc3c12 ; Brc1cccc(-c2nc(-c3ccccc3)nc(-c3cccc4c3oc3ccccc34)n2)c1 ; Clc1ccc2oc3ccccc3c2c1-c1cccc(-c2nc(-c3ccccc3)nc(-c3cccc4c3oc3ccccc34)n2)c1 ; OB(O)c1ccccc1]

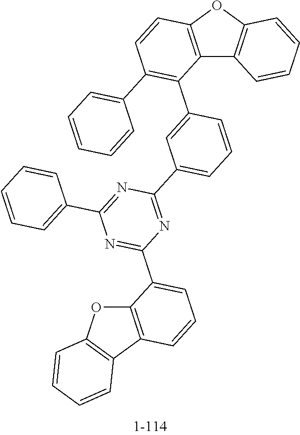 [SMILES: c1ccc(-c2nc(-c3cccc(-c4c(-c5ccccc5)ccc5oc6ccccc6c45)c3)nc(-c3cccc4c3oc3ccccc34)n2)cc1]

(2-Chlorodibenzo[b,d]furan-1-yl)boronic acid (15 g, 60.9 mmol) and Trz87 (30.6 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred, and then bis(tri-tert-butylphosphine)palladium(0) (0.3 g, 0.6 mmol) was added. After the reaction for 5 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 27 g of Compound 1-114_P1. (Yield: 74%, MS: [M+H]+=600)

Compound 1-114_P1 (15 g, 25 mmol) and phenylboronic acid (3.2 g, 26.2 mmol) were added to 300 ml of THF, and the mixture was stirred and refluxed. Then, potassium carbonate (10.4 g, 75 mmol) was dissolved in 31 ml of water and added thereto, and the mixture was sufficiently stirred, and then bis(tri-tert-butylphosphine)palladium(0) (0.1 g, 0.2 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11.5 g of Compound 1-114. (Yield: 72%, MS: [M+H]+=642)

### Preparation Example 1-115

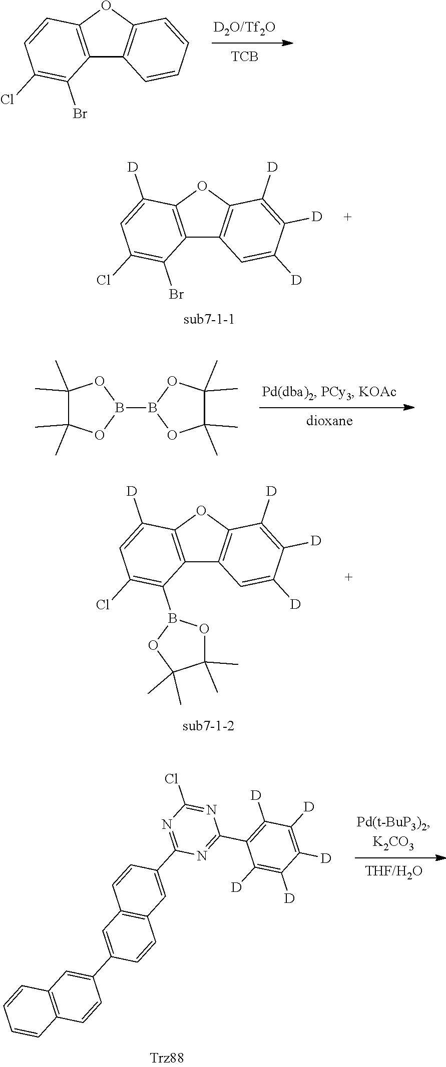

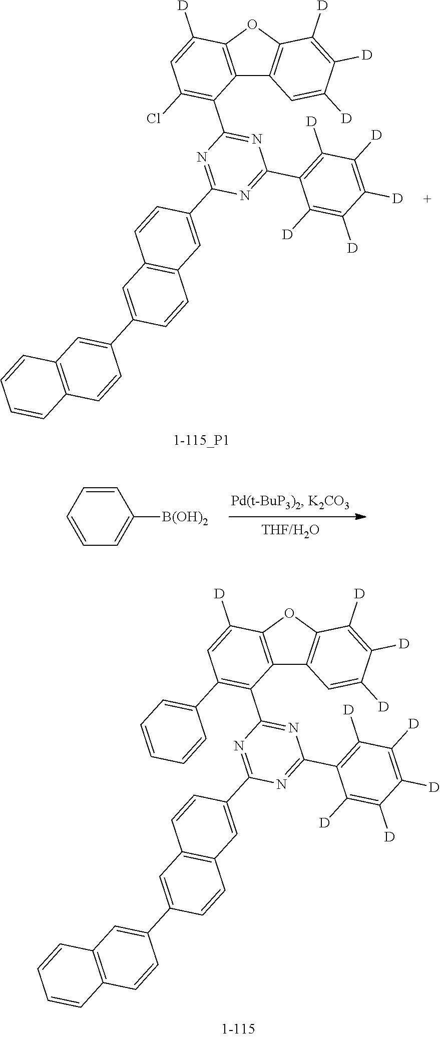 [SMILES: [2H]c1cc2c(oc3c([2H])cc(Cl)c(-c4nc(-c5ccc6cc(-c7ccc8ccccc8c7)ccc6c5)nc(-c5c([2H])c([2H])c([2H])c([2H])c5[2H])n4)c32)c([2H])c1[2H] ; OB(O)c1ccccc1 ; [2H]c1cc2c(oc3c([2H])cc(-c4ccccc4)c(-c4nc(-c5ccc6cc(-c7ccc8ccccc8c7)ccc6c5)nc(-c5c([2H])c([2H])c([2H])c([2H])c5[2H])n4)c32)c([2H])c1[2H]]

Trifluoromethanesulfonic anhydride (60.1 g, 213.1 mmol) and deuterium oxide (21.4 g, 1065.6 mmol) were added at 0° C. and stirred for 5 hours to prepare a solution. 1-Bromo-2-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-2-chlorodibenzo[b,d]furan and 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.1 g of sub7-1-1. (Yield: 40%, MS: [M+H]+=285)

Sub7-1-1 (15 g, 52.5 mmol) and bis(pinacolato)diboron (14.7 g, 57.8 mmol) were added to 300 ml of 1,4-dioxane, and the mixture was stirred and refluxed. Then, potassium acetate (7.7 g, 78.8 mmol) was added thereto, and the mixture was sufficiently stirred, and then bis(dibenzylideneacetone)palladium(0) (0.9 g, 1.6 mmol) and tricyclohexylphosphine (0.9 g, 3.2 mmol) were added. After the reaction for 5 hours, the reaction mixture was cooled to room temperature, and then the organic layer was separated using chloroform and water, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 10.5 g of sub7-1-2. (Yield: 60%, MS: [M+H]+=333)

Sub7-1-2 (15 g, 45.1 mmol) and Trz88 (21.3 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred, and then bis(tri-tert-butylphosphine)palladium(0) (0.2 g, 0.5 mmol) was added. After the reaction for 4 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 18.1 g of Compound 1-115_P1. (Yield: 65%, MS: [M+H]+=619)

Compound 1-115_P1 (15 g, 24.2 mmol) and phenylboronic acid (3.1 g, 25.4 mmol) were added to 300 ml of THF, and the mixture was stirred and refluxed. Then, potassium carbonate (10 g, 72.7 mmol) was dissolved in 30 ml of water and added thereto, and the mixture was sufficiently stirred, and then bis(tri-tert-butylphosphine)palladium(0) (0.1 g, 0.2 mmol) was added. After the reaction for 3 hours, the reaction mixture was cooled to room temperature, and then the organic layer and the aqueous layer were separated, and the organic layer was distilled. This was dissolved again in chloroform and washed twice with water. The organic layer was then separated, anhydrous magnesium sulfate was added, stirred, then filtered, and the filtrate was distilled under reduced pressure. The concentrated compound was purified by a silica gel column chromatography to give 11 g of Compound 1-115. (Yield: 69%, MS: [M+H]+=661)

### Preparation Example 1-116

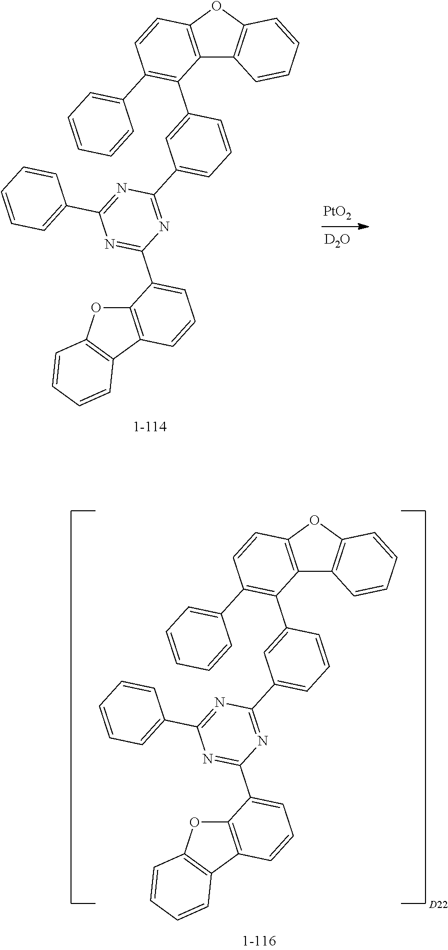

Compound 1-114 (10 g, 15.6 mmol), PtO₂ (1.1 g, 4.7 mmol) and D₂O (78 ml) were added to a shaker tube, and then the tube was sealed and heated at 250° C. and 600 psi for 12 hours. When the reaction was completed, chloroform was added, and the reaction solution was transferred to a separatory funnel, and extracted. The extract was dried over MgSO₄ and concentrated, and then the sample was purified by silica gel column chromatography to prepare 4.3 g of Compound 1-116. (Yield: 42%, MS: [M+H]+=665)

### Preparation Example 2: Preparation of Compound of Chemical Formula 2

### Preparation Example 2-1

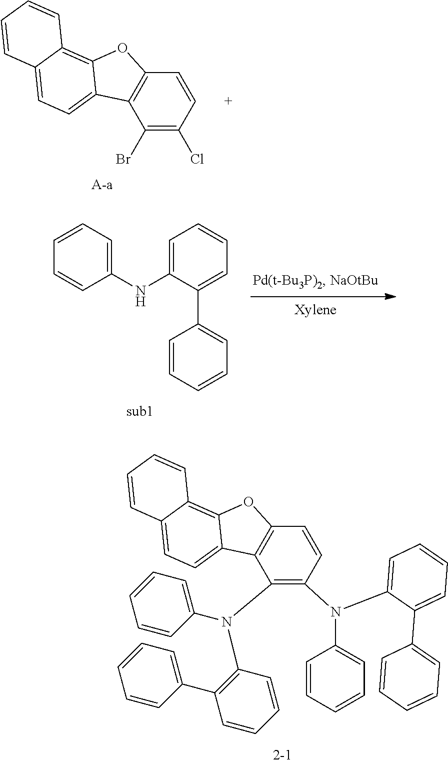 [SMILES: Clc1ccc2oc3c4ccccc4ccc3c2c1Br ; c1ccc(Nc2ccccc2-c2ccccc2)cc1 ; c1ccc(-c2ccccc2N(c2ccccc2)c2ccc3oc4c5ccccc5ccc4c3c2N(c2ccccc2)c2ccccc2-c2ccccc2)cc1]

Compound A-a (10 g, 30.2 mmol), sub1 (15.2 g, 61.8 mmol) and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.4 g of Compound 2-1. (Yield: 68%, MS: [M+H]+=705)

### Preparation Example 2-2

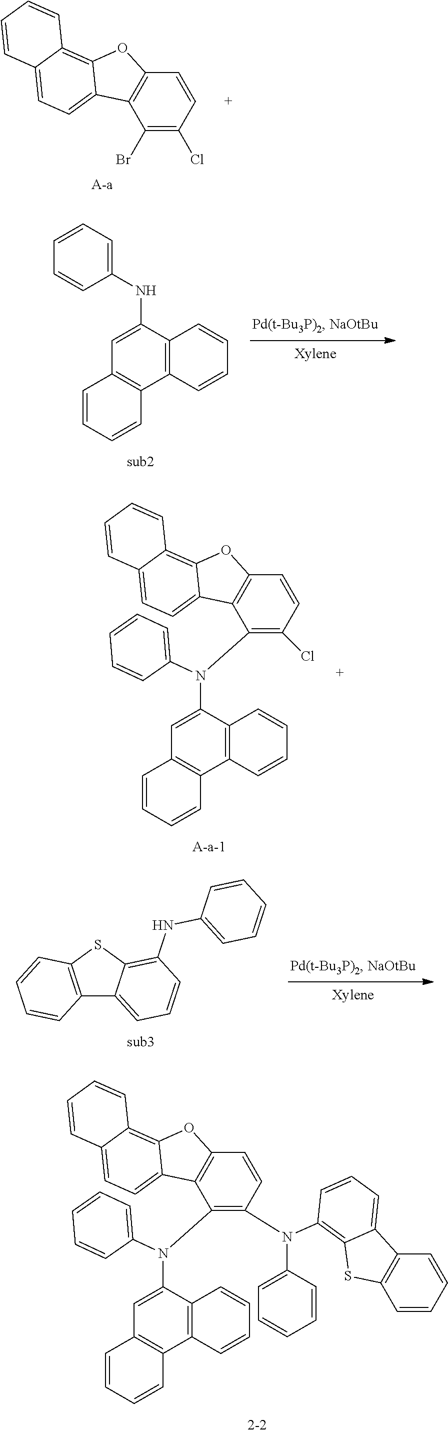 [SMILES: Clc1ccc2oc3c4ccccc4ccc3c2c1Br ; c1ccc(Nc2cc3ccccc3c3ccccc23)cc1 ; Clc1ccc2oc3c4ccccc4ccc3c2c1N(c1ccccc1)c1cc2ccccc2c2ccccc12 ; c1ccc(Nc2cccc3c2sc2ccccc23)cc1 ; c1ccc(N(c2ccc3oc4c5ccccc5ccc4c3c2N(c2ccccc2)c2cc3ccccc3c3ccccc23)c2cccc3c2sc2ccccc23)cc1]

Compound A-a (10 g, 30.2 mmol), sub2 (8.2 g, 30.5 mmol) and sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.4 g of Compound A-a-1. (Yield: 60%, MS: [M+H]+=520)

Compound A-a-1 (10 g, 19.2 mmol), sub3 (5.4 g, 19.6 mmol) and sodium tert-butoxide (2.4 g, 25 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.6 g of Compound 2-2. (Yield: 52%, MS: [M+H]+=759)

### Preparation Example 2-3

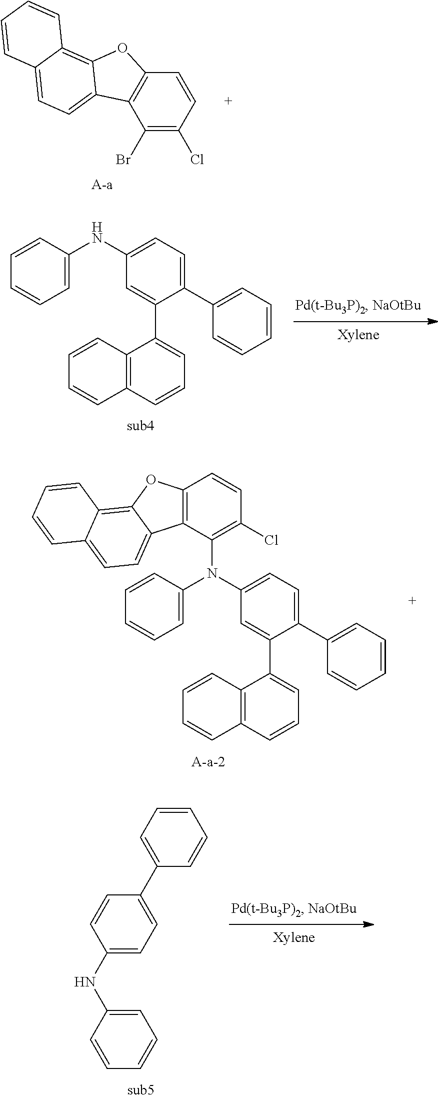 [SMILES: Clc1ccc2oc3c4ccccc4ccc3c2c1Br ; c1ccc(Nc2ccc(-c3ccccc3)c(-c3cccc4ccccc34)c2)cc1 ; Clc1ccc2oc3c4ccccc4ccc3c2c1N(c1ccccc1)c1ccc(-c2ccccc2)c(-c2cccc3ccccc23)c1 ; c1ccc(Nc2ccc(-c3ccccc3)cc2)cc1]

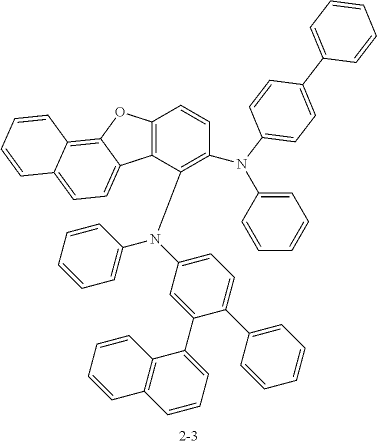 [SMILES: c1ccc(-c2ccc(N(c3ccccc3)c3ccc4oc5c6ccccc6ccc5c4c3N(c3ccccc3)c3ccc(-c4ccccc4)c(-c4cccc5ccccc45)c3)cc2)cc1]

Compound A-a (10 g, 30.2 mmol), sub4 (11.8 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.5 g of Compound A-a-2. (Yield: 72%, MS: [M+H]+=622)

Compound A-a-2 (10 g, 16.1 mmol), sub5 (4.1 g, 16.9 mmol) and sodium tert-butoxide (2 g, 20.9 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.4 g of Compound 2-3. (Yield: 63%, MS: [M+H]+=831)

### Preparation Example 2-4

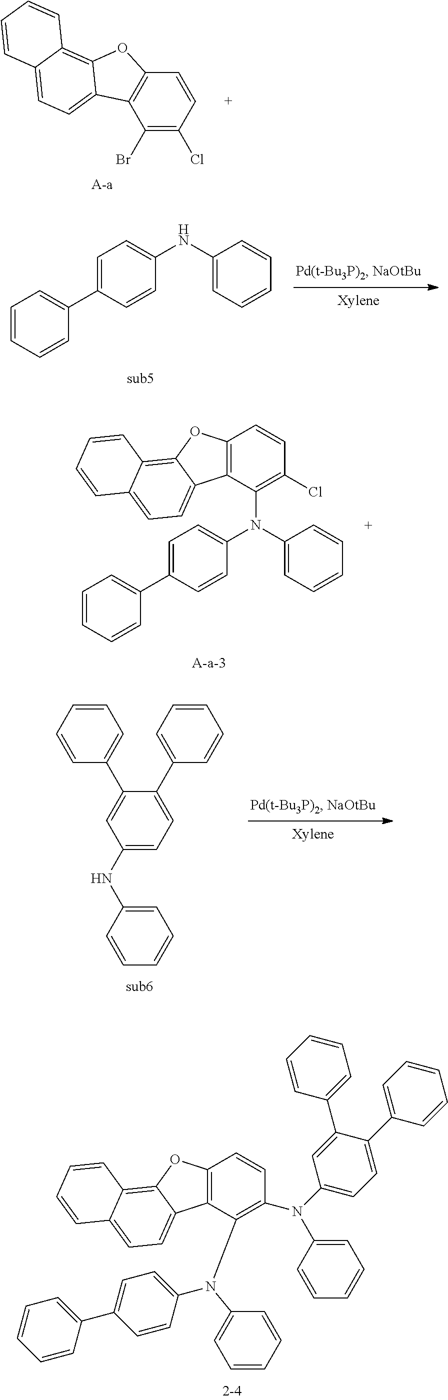 [SMILES: Clc1ccc2oc3c4ccccc4ccc3c2c1Br ; c1ccc(Nc2ccc(-c3ccccc3)cc2)cc1 ; Clc1ccc2oc3c4ccccc4ccc3c2c1N(c1ccccc1)c1ccc(-c2ccccc2)cc1 ; c1ccc(Nc2ccc(-c3ccccc3)c(-c3ccccc3)c2)cc1 ; c1ccc(-c2ccc(N(c3ccccc3)c3c(N(c4ccccc4)c4ccc(-c5ccccc5)c(-c5ccccc5)c4)ccc4oc5c6ccccc6ccc5c34)cc2)cc1]

Compound A-a (10 g, 30.2 mmol), sub5 (7.8 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.7 g of Compound A-a-3. (Yield: 65%, MS: [M+H]+=496)

Compound A-a-3 (10 g, 20.2 mmol), sub6 (6.8 g, 21.2 mmol) and sodium tert-butoxide (2.5 g, 26.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.2 g of Compound 2-4. (Yield: 65%, MS: [M+H]+=781)

### Preparation Example 2-5

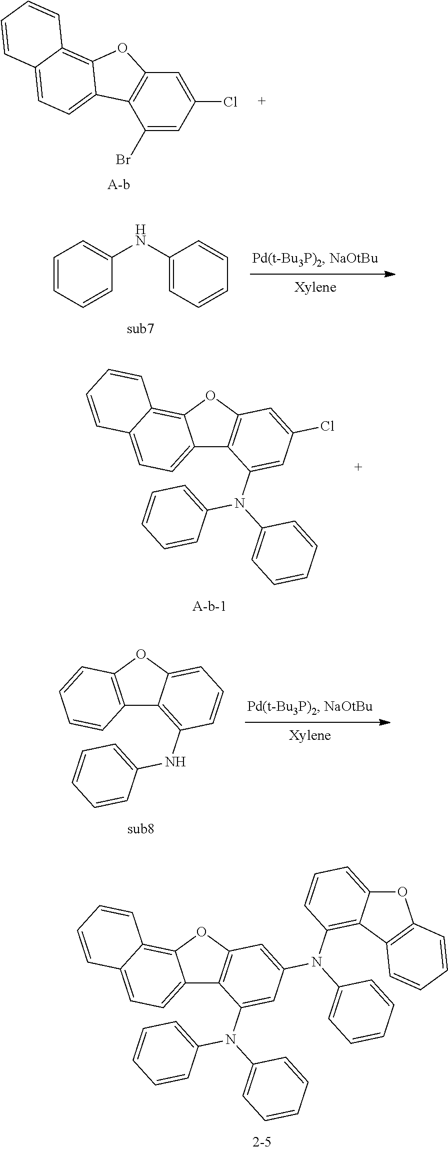 [SMILES: Clc1cc(Br)c2c(c1)oc1c3ccccc3ccc12 ; c1ccc(Nc2ccccc2)cc1 ; Clc1cc(N(c2ccccc2)c2ccccc2)c2c(c1)oc1c3ccccc3ccc12 ; c1ccc(Nc2cccc3oc4ccccc4c23)cc1 ; c1ccc(N(c2cc(N(c3ccccc3)c3ccccc3)c3c(c2)oc2c4ccccc4ccc23)c2cccc3oc4ccccc4c23)cc1]

Compound A-b (10 g, 30.2 mmol), sub7 (5.2 g, 30.5 mmol) and sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.1 g of Compound A-b-1. (Yield: 56%, MS: [M+H]+=420)

Compound A-b-1 (10 g, 23.8 mmol), sub8 (6.3 g, 24.3 mmol) and sodium tert-butoxide (3 g, 31 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.8 g of Compound 2-5. (Yield: 51%, MS: [M+H]+=643)

### Preparation Example 2-6

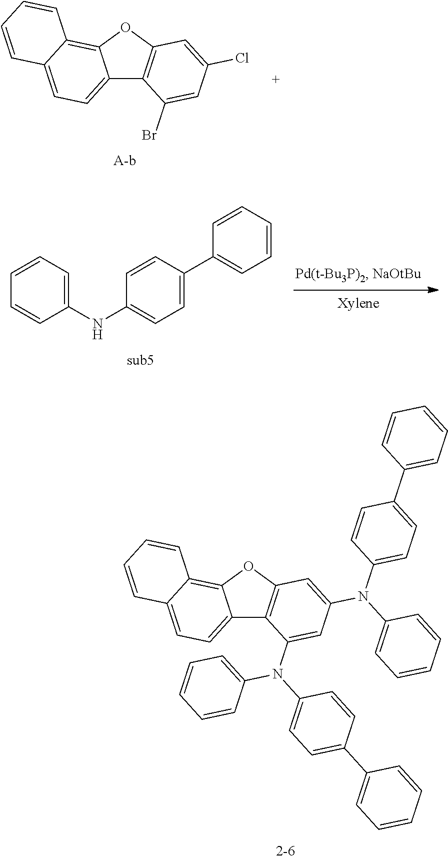 [SMILES: Clc1cc(Br)c2c(c1)oc1c3ccccc3ccc12 ; c1ccc(Nc2ccc(-c3ccccc3)cc2)cc1 ; c1ccc(-c2ccc(N(c3ccccc3)c3cc(N(c4ccccc4)c4ccc(-c5ccccc5)cc4)c4c(c3)oc3c5ccccc5ccc34)cc2)cc1]

Compound A-b (10 g, 30.2 mmol), sub5 (15.2 g, 61.8 mmol) and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11 g of Compound 2-6. (Yield: 52%, MS: [M+H]+=705)

### Preparation Example 2-7

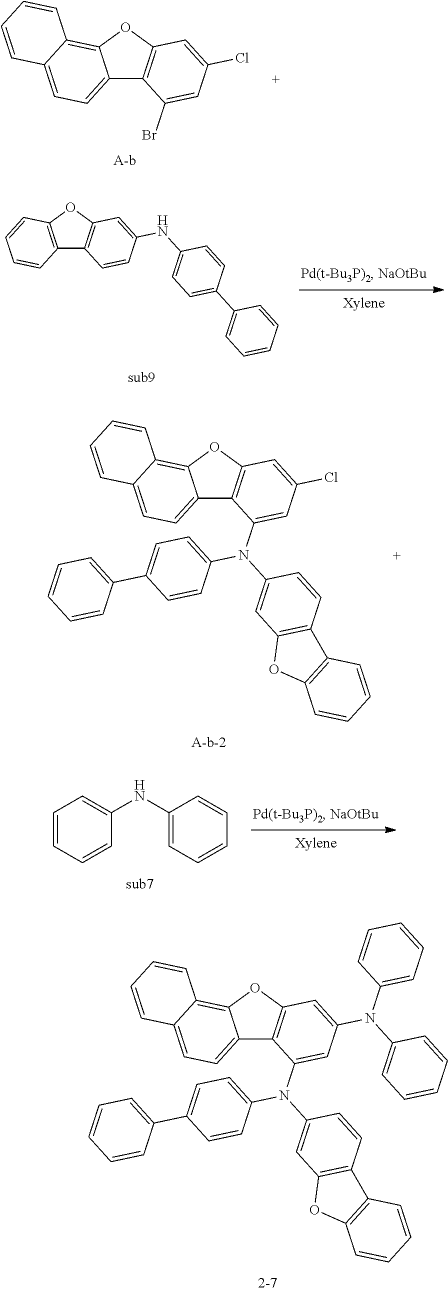 [SMILES: Clc1cc(Br)c2c(c1)oc1c3ccccc3ccc12 ; c1ccc(-c2ccc(Nc3ccc4c(c3)oc3ccccc34)cc2)cc1 ; Clc1cc(N(c2ccc(-c3ccccc3)cc2)c2ccc3c(c2)oc2ccccc23)c2c(c1)oc1c3ccccc3ccc12 ; c1ccc(Nc2ccccc2)cc1 ; c1ccc(-c2ccc(N(c3ccc4c(c3)oc3ccccc34)c3cc(N(c4ccccc4)c4ccccc4)cc4oc5c6ccccc6ccc5c34)cc2)cc1]

Compound A-b (10 g, 30.2 mmol), sub9 (10.2 g, 30.5 mmol) and sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.8 g of Compound A-b-2. (Yield: 61%, MS: [M+H]+=586)

Compound A-b-2 (10 g, 17.1 mmol), sub7 (2.9 g, 17.4 mmol) and sodium tert-butoxide (2.1 g, 22.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.4 g of Compound 2-7. (Yield: 60%, MS: [M+H]+=719)

### Preparation Example 2-8

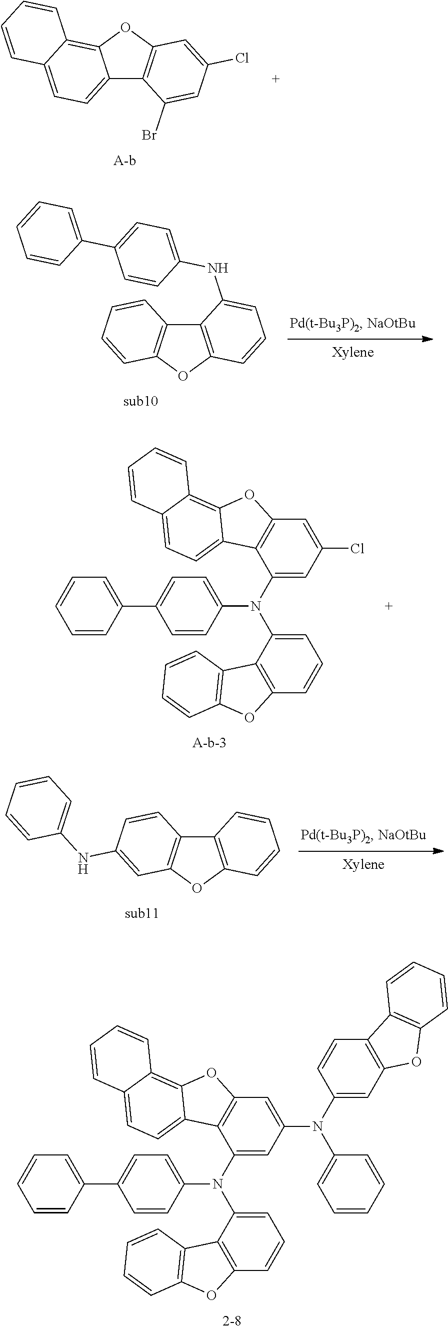 [SMILES: Clc1cc(Br)c2c(c1)oc1c3ccccc3ccc12 ; c1ccc(-c2ccc(Nc3cccc4oc5ccccc5c34)cc2)cc1 ; Clc1cc(N(c2ccc(-c3ccccc3)cc2)c2cccc3oc4ccccc4c23)c2c(c1)oc1c3ccccc3ccc12 ; c1ccc(Nc2ccc3c(c2)oc2ccccc23)cc1 ; c1ccc(-c2ccc(N(c3cccc4oc5ccccc5c34)c3cc(N(c4ccccc4)c4ccc5c(c4)oc4ccccc45)cc4oc5c6ccccc6ccc5c34)cc2)cc1]

Compound A-b (10 g, 30.2 mmol), sub10 (10.6 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.8 g of Compound A-b-3. (Yield: 67%, MS: [M+H]+=586)

Compound A-b-3 (10 g, 17.1 mmol), sub11 (4.6 g, 17.9 mmol) and sodium tert-butoxide (2.1 g, 22.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.3 g of Compound 2-8. (Yield: 60%, MS: [M+H]+=809)

### Preparation Example 2-9

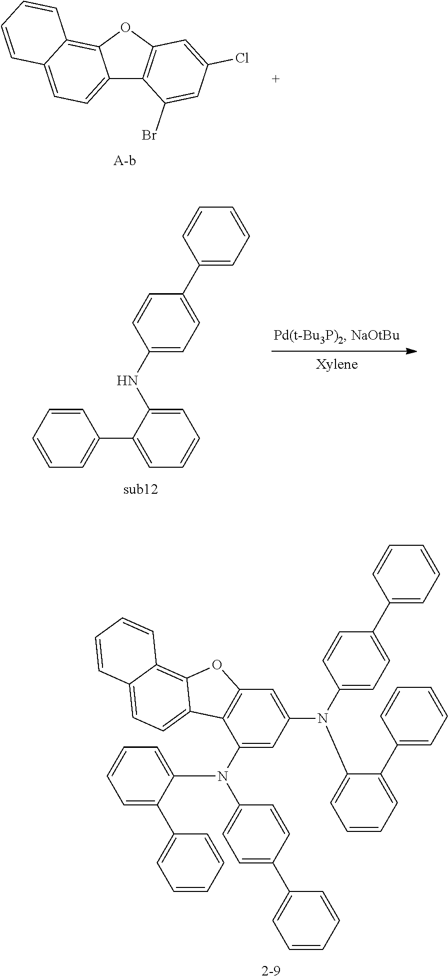 [SMILES: Clc1cc(Br)c2c(c1)oc1c3ccccc3ccc12 ; c1ccc(-c2ccc(Nc3ccccc3-c3ccccc3)cc2)cc1 ; c1ccc(-c2ccc(N(c3cc(N(c4ccc(-c5ccccc5)cc4)c4ccccc4-c4ccccc4)c4c(c3)oc3c5ccccc5ccc34)c3ccccc3-c3ccccc3)cc2)cc1]

Compound A-b (10 g, 30.2 mmol), sub12 (19.9 g, 61.8 mmol) and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 17.6 g of Compound 2-9. (Yield: 68%, MS: [M+H]+=857)

### Preparation Example 2-10

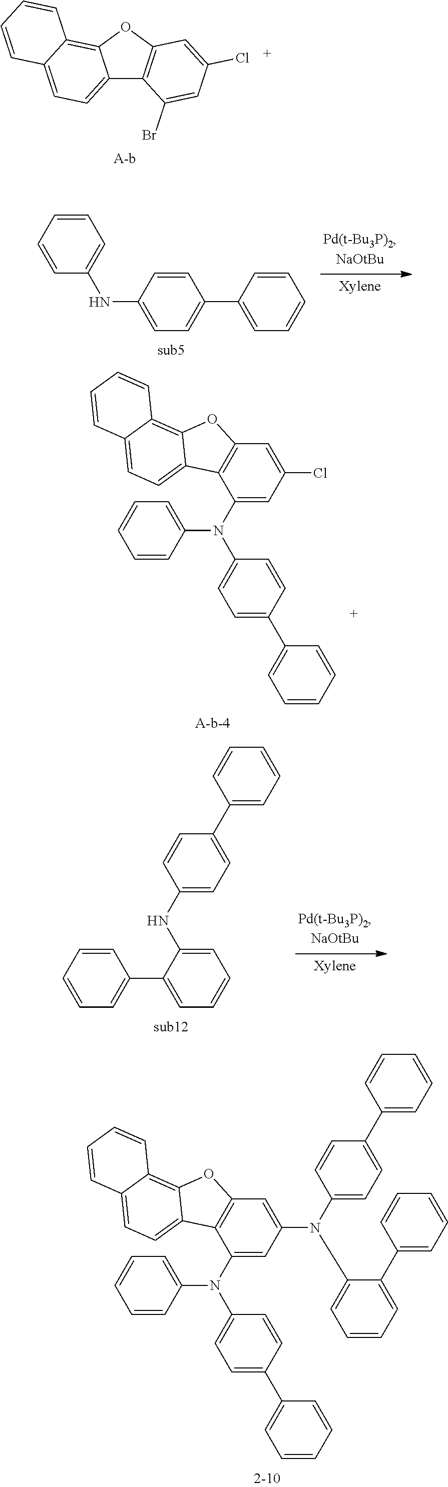 [SMILES: Clc1cc(Br)c2c(c1)oc1c3ccccc3ccc12 ; c1ccc(Nc2ccc(-c3ccccc3)cc2)cc1 ; Clc1cc(N(c2ccccc2)c2ccc(-c3ccccc3)cc2)c2c(c1)oc1c3ccccc3ccc12 ; c1ccc(-c2ccc(Nc3ccccc3-c3ccccc3)cc2)cc1 ; c1ccc(-c2ccc(N(c3cc(N(c4ccccc4)c4ccc(-c5ccccc5)cc4)c4c(c3)oc3c5ccccc5ccc34)c3ccccc3-c3ccccc3)cc2)cc1]

Compound A-b (10 g, 30.2 mmol), sub5 (7.8 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.3 g of Compound A-b-4. (Yield: 62%, MS: [M+H]+=496)

Compound A-b-4 (10 g, 20.2 mmol), sub12 (6.8 g, 21.2 mmol) and sodium tert-butoxide (2.5 g, 26.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.4 g of Compound 2-10. (Yield: 60%, MS: [M+H]+=781)

### Preparation Example 2-11

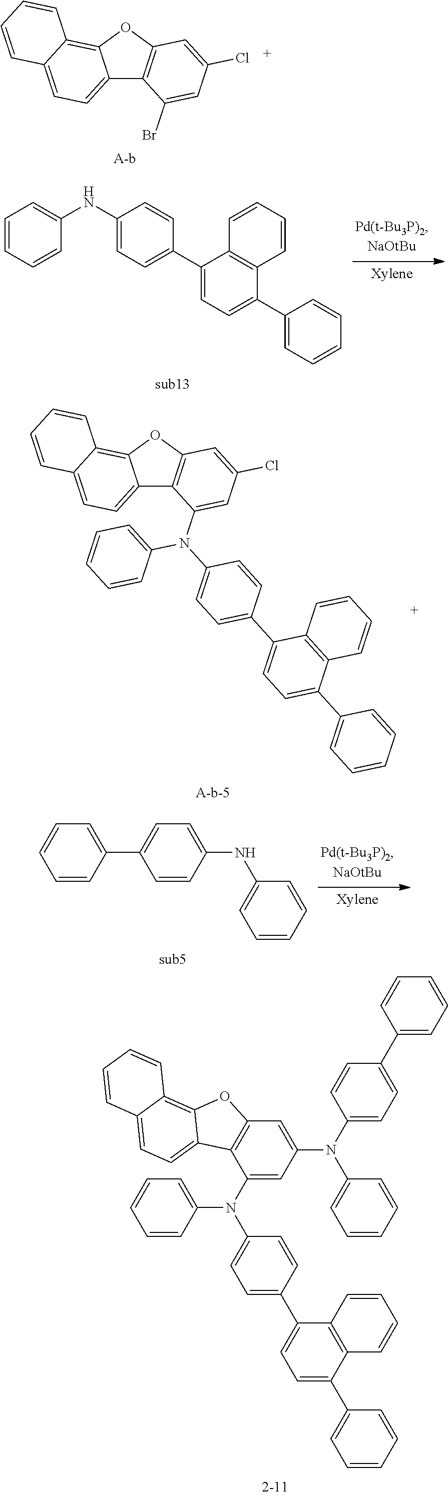 [SMILES: Clc1cc(Br)c2c(c1)oc1c3ccccc3ccc12 ; c1ccc(Nc2ccc(-c3ccc(-c4ccccc4)c4ccccc34)cc2)cc1 ; Clc1cc(N(c2ccccc2)c2ccc(-c3ccc(-c4ccccc4)c4ccccc34)cc2)c2c(c1)oc1c3ccccc3ccc12 ; c1ccc(Nc2ccc(-c3ccccc3)cc2)cc1 ; c1ccc(-c2ccc(N(c3ccccc3)c3cc(N(c4ccccc4)c4ccc(-c5ccc(-c6ccccc6)c6ccccc56)cc4)c4c(c3)oc3c5ccccc5ccc34)cc2)cc1]

Compound A-b (10 g, 30.2 mmol), sub13 (11.8 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.6 g of Compound A-b-5. (Yield: 62%, MS: [M+H]+=622)

Compound A-b-5 (10 g, 16.1 mmol), sub5 (4.1 g, 16.9 mmol) and sodium tert-butoxide (2 g, 20.9 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.1 g of Compound 2-11. (Yield: 61%, MS: [M+H]+=831)

### Preparation Example 2-12

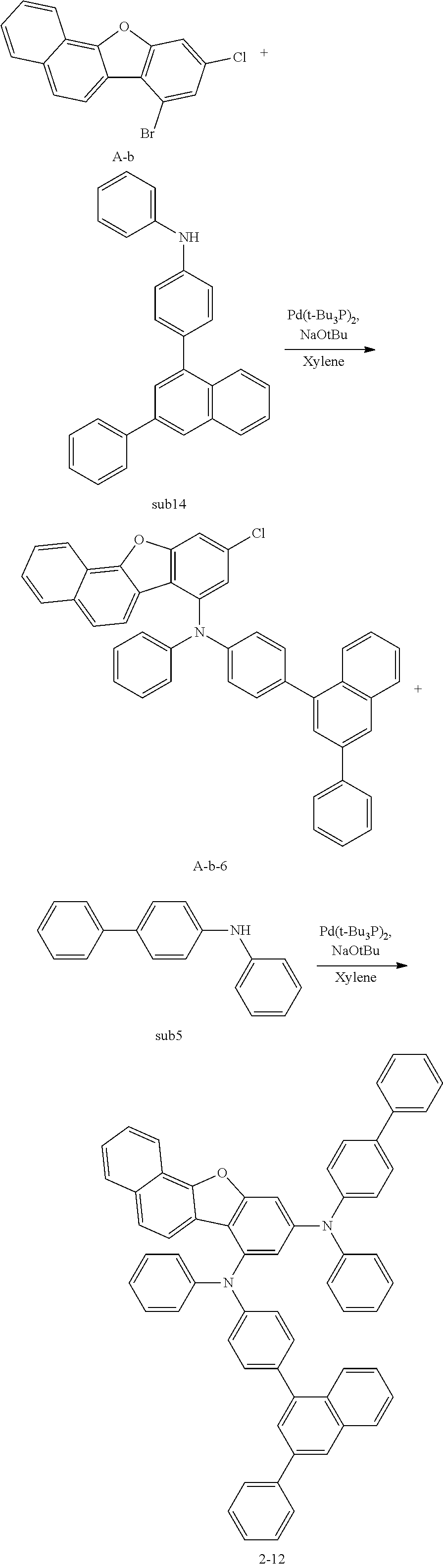 [SMILES: Clc1cc(Br)c2c(c1)oc1c3ccccc3ccc12 ; c1ccc(Nc2ccc(-c3cc(-c4ccccc4)cc4ccccc34)cc2)cc1 ; Clc1cc(N(c2ccccc2)c2ccc(-c3cc(-c4ccccc4)cc4ccccc34)cc2)c2c(c1)oc1c3ccccc3ccc12 ; c1ccc(Nc2ccc(-c3ccccc3)cc2)cc1 ; c1ccc(-c2ccc(N(c3ccccc3)c3cc(N(c4ccccc4)c4ccc(-c5cc(-c6ccccc6)cc6ccccc56)cc4)c4c(c3)oc3c5ccccc5ccc34)cc2)cc1]

Compound A-b (10 g, 30.2 mmol), sub14 (11.8 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.1 g of Compound A-b-6. (Yield: 70%, MS: [M+H]+=622)

Compound A-b-6 (10 g, 16.1 mmol), sub5 (4.1 g, 16.9 mmol) and sodium tert-butoxide (2 g, 20.9 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8 g of Compound 2-12. (Yield: 60%, MS: [M+H]+=831)

### Preparation Example 2-13

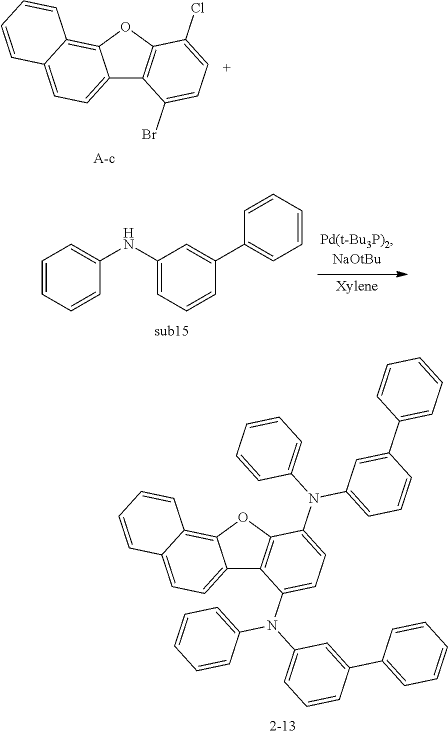 [SMILES: Clc1ccc(Br)c2c1oc1c3ccccc3ccc12 ; c1ccc(Nc2cccc(-c3ccccc3)c2)cc1 ; c1ccc(-c2cccc(N(c3ccccc3)c3ccc(N(c4ccccc4)c4cccc(-c5ccccc5)c4)c4c3oc3c5ccccc5ccc34)c2)cc1]

Compound A-c (10 g, 30.2 mmol), sub15 (15.2 g, 61.8 mmol) and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.7 g of Compound 2-13. (Yield: 69%, MS: [M+H]+=705)

### Preparation Example 2-14

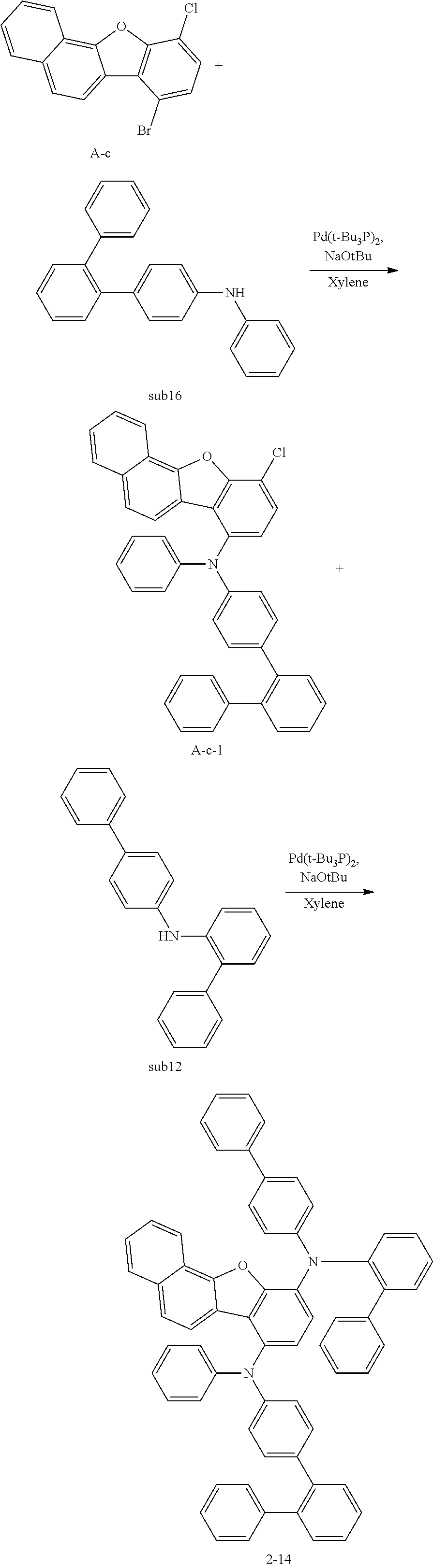 [SMILES: Clc1ccc(Br)c2c1oc1c3ccccc3ccc12 ; c1ccc(Nc2ccc(-c3ccccc3-c3ccccc3)cc2)cc1 ; Clc1ccc(N(c2ccccc2)c2ccc(-c3ccccc3-c3ccccc3)cc2)c2c1oc1c3ccccc3ccc12 ; c1ccc(-c2ccc(Nc3ccccc3-c3ccccc3)cc2)cc1 ; c1ccc(-c2ccc(N(c3ccccc3-c3ccccc3)c3ccc(N(c4ccccc4)c4ccc(-c5ccccc5-c5ccccc5)cc4)c4c3oc3c5ccccc5ccc34)cc2)cc1]

Compound A-c (10 g, 30.2 mmol), sub16 (10.2 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.7 g of Compound A-c-1. (Yield: 62%, MS: [M+H]+=572)

Compound A-c-1 (10 g, 17.5 mmol), sub12 (5.9 g, 18.4 mmol) and sodium tert-butoxide (2.2 g, 22.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9 g of Compound 2-14. (Yield: 60%, MS: [M+H]+=857)

### Preparation Example 2-15

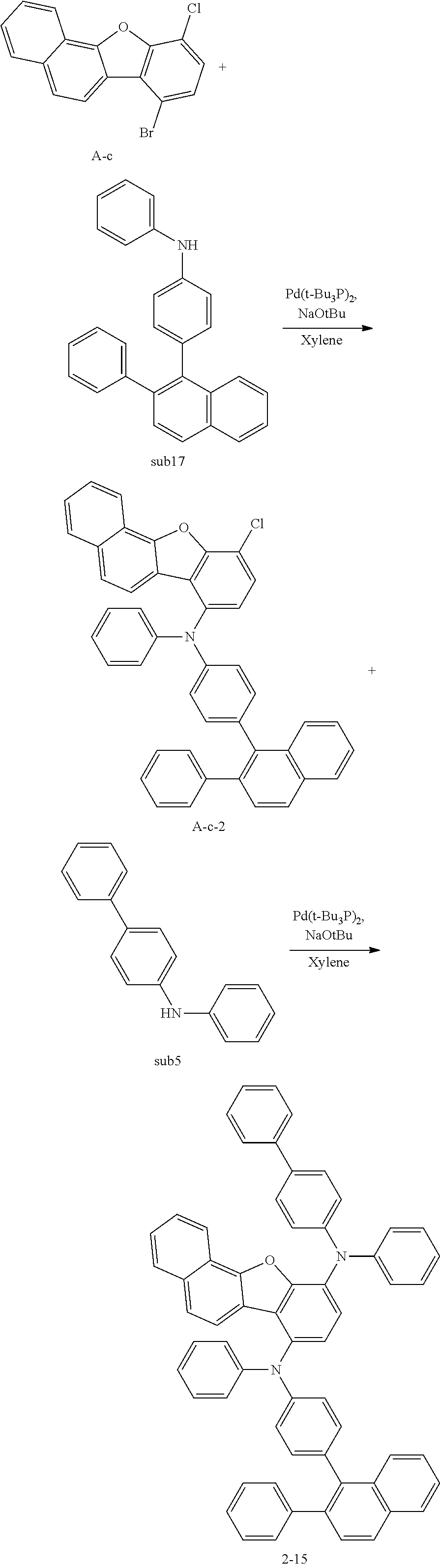 [SMILES: Clc1ccc(Br)c2c1oc1c3ccccc3ccc12 ; c1ccc(Nc2ccc(-c3c(-c4ccccc4)ccc4ccccc34)cc2)cc1 ; Clc1ccc(N(c2ccccc2)c2ccc(-c3c(-c4ccccc4)ccc4ccccc34)cc2)c2c1oc1c3ccccc3ccc12 ; c1ccc(Nc2ccc(-c3ccccc3)cc2)cc1 ; c1ccc(-c2ccc(N(c3ccccc3)c3ccc(N(c4ccccc4)c4ccc(-c5c(-c6ccccc6)ccc6ccccc56)cc4)c4c3oc3c5ccccc5ccc34)cc2)cc1]

Compound A-c (10 g, 30.2 mmol), sub17 (11.8 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.6 g of Compound A-c-2. (Yield: 62%, MS: [M+H]+=622)

Compound A-c-2 (10 g, 16.1 mmol), sub5 (4.1 g, 16.9 mmol) and sodium tert-butoxide (2 g, 20.9 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.1 g of Compound 2-15. (Yield: 68%, MS: [M+H]+=831)

### Preparation Example 2-16

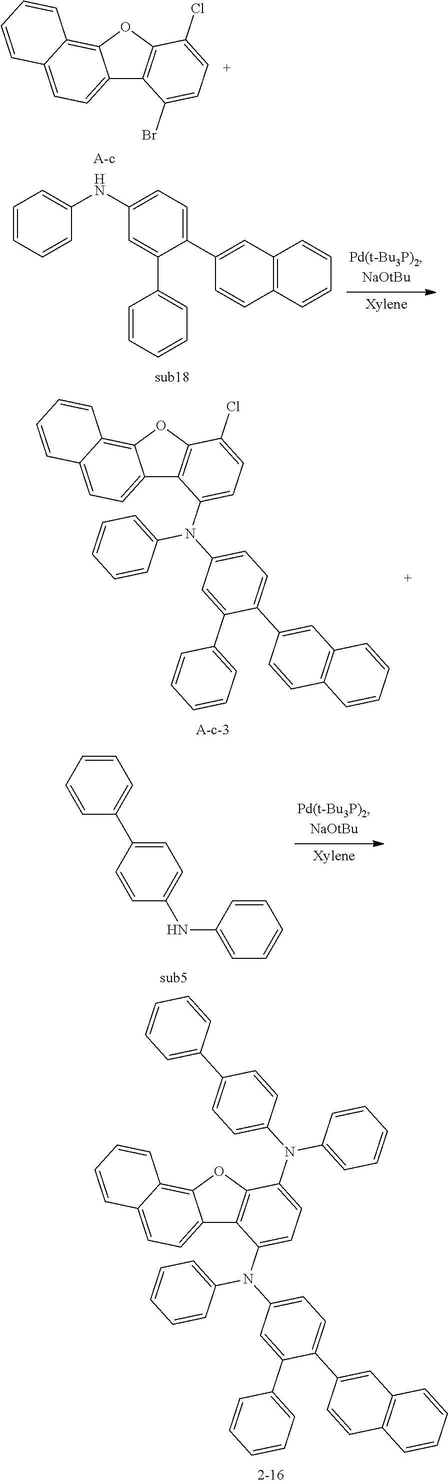 [SMILES: Clc1ccc(Br)c2c1oc1c3ccccc3ccc12 ; c1ccc(Nc2ccc(-c3ccc4ccccc4c3)c(-c3ccccc3)c2)cc1 ; Clc1ccc(N(c2ccccc2)c2ccc(-c3ccc4ccccc4c3)c(-c3ccccc3)c2)c2c1oc1c3ccccc3ccc12 ; c1ccc(Nc2ccc(-c3ccccc3)cc2)cc1 ; c1ccc(-c2ccc(N(c3ccccc3)c3ccc(N(c4ccccc4)c4ccc(-c5ccc6ccccc6c5)c(-c5ccccc5)c4)c4c3oc3c5ccccc5ccc34)cc2)cc1]

Compound A-c (10 g, 30.2 mmol), sub18 (11.8 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.5 g of Compound A-c-3. (Yield: 72%, MS: [M+H]+=622)

Compound A-c-3 (10 g, 16.1 mmol), sub5 (4.1 g, 16.9 mmol) and sodium tert-butoxide (2 g, 20.9 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.9 g of Compound 2-16. (Yield: 67%, MS: [M+H]+=831)

### Preparation Example 2-17

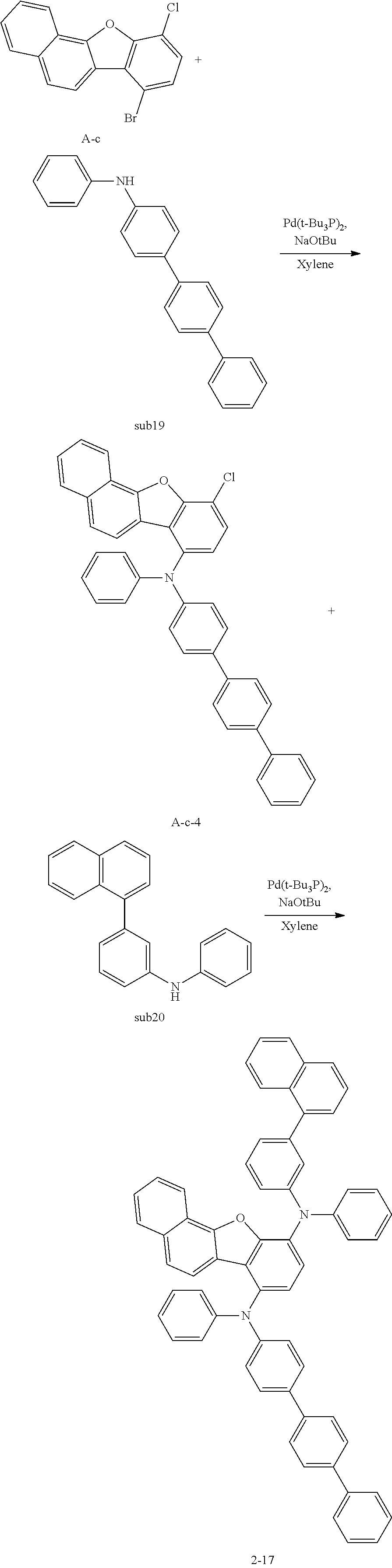 [SMILES: Clc1ccc(Br)c2c1oc1c3ccccc3ccc12 ; c1ccc(Nc2ccc(-c3ccc(-c4ccccc4)cc3)cc2)cc1 ; Clc1ccc(N(c2ccccc2)c2ccc(-c3ccc(-c4ccccc4)cc3)cc2)c2c1oc1c3ccccc3ccc12 ; c1ccc(Nc2cccc(-c3cccc4ccccc34)c2)cc1 ; c1ccc(-c2ccc(-c3ccc(N(c4ccccc4)c4ccc(N(c5ccccc5)c5cccc(-c6cccc7ccccc67)c5)c5oc6c7ccccc7ccc6c45)cc3)cc2)cc1]

Compound A-c (10 g, 30.2 mmol), sub19 (10.2 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.7 g of Compound A-c-4. (Yield: 62%, MS: [M+H]+=572)

Compound A-c-4 (10 g, 17.5 mmol), sub20 (5.4 g, 18.4 mmol) and sodium tert-butoxide (2.2 g, 22.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.9 g of Compound 2-17. (Yield: 75%, MS: [M+H]+=831)

### Preparation Example 2-18

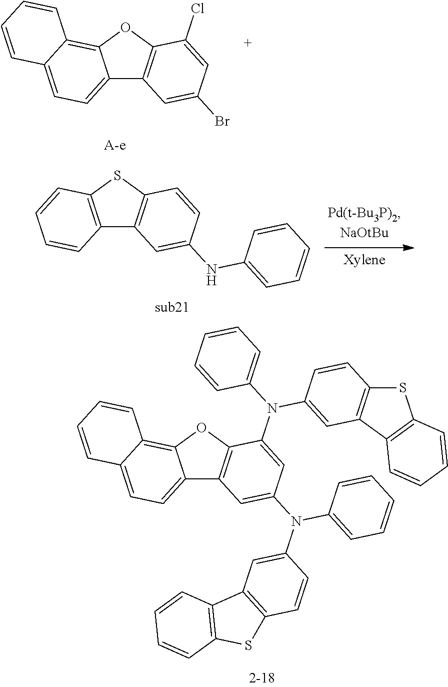 [SMILES: Clc1cc(Br)cc2c1oc1c3ccccc3ccc21 ; c1ccc(Nc2ccc3sc4ccccc4c3c2)cc1 ; c1ccc(N(c2ccc3sc4ccccc4c3c2)c2cc(N(c3ccccc3)c3ccc4sc5ccccc5c4c3)c3oc4c5ccccc5ccc4c3c2)cc1]

Compound A-e (10 g, 30.2 mmol), sub21 (17 g, 61.8 mmol) and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.3 g of Compound 2-18. (Yield: 62%, MS: [M+H]+=765)

### Preparation Example 2-19

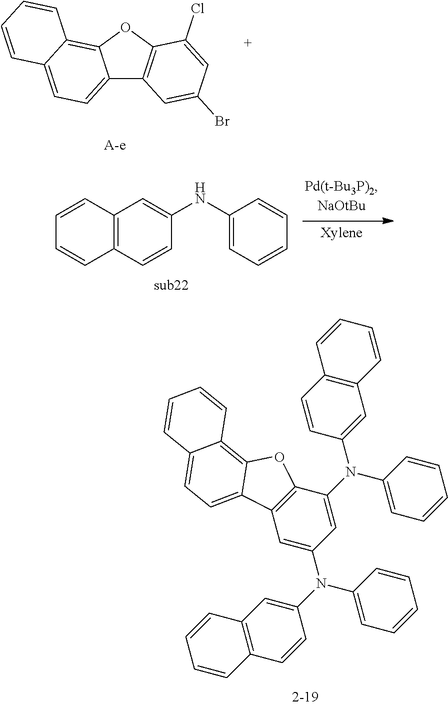 [SMILES: Clc1cc(Br)cc2c1oc1c3ccccc3ccc21 ; c1ccc(Nc2ccc3ccccc3c2)cc1 ; c1ccc(N(c2ccc3ccccc3c2)c2cc(N(c3ccccc3)c3ccc4ccccc4c3)c3oc4c5ccccc5ccc4c3c2)cc1]

Compound A-e (10 g, 30.2 mmol), sub22 (13.6 g, 61.8 mmol) and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.4 g of Compound 2-19. (Yield: 63%, MS: [M+H]+=653)

### Preparation Example 2-20

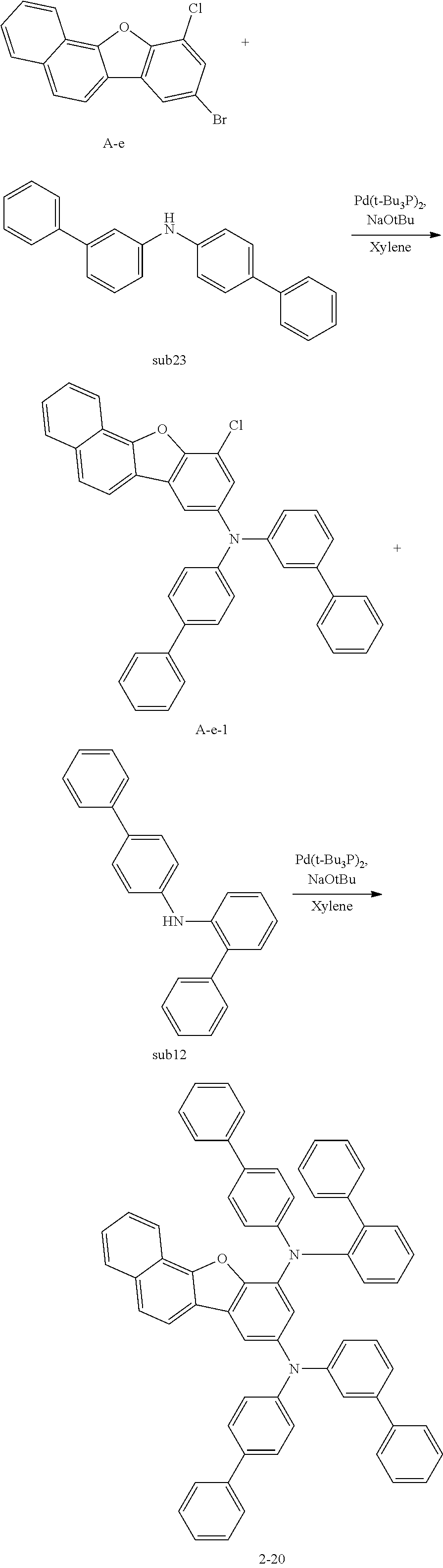

Compound A-e (10 g, 30.2 mmol), sub23 (10.2 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.7 g of Compound A-e-1. (Yield: 62%, MS: [M+H]+=572)

Compound A-e-1 (10 g, 17.5 mmol), sub12 (5.9 g, 18.4 mmol) and sodium tert-butoxide (2.2 g, 22.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.3 g of Compound 2-20. (Yield: 62%, MS: [M+H]+=857)

### Preparation Example 2-21

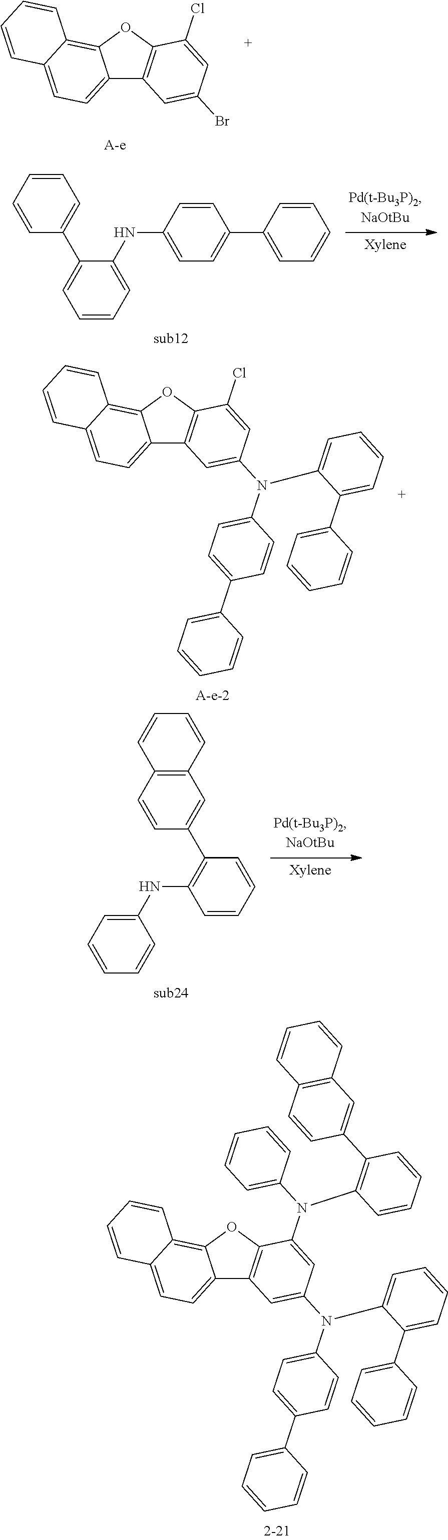

Compound A-e (10 g, 30.2 mmol), sub12 (10.2 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.9 g of Compound A-e-2. (Yield: 63%, MS: [M+H]+=572)

Compound A-e-2 (10 g, 17.5 mmol), sub24 (5.4 g, 18.4 mmol) and sodium tert-butoxide (2.2 g, 22.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.6 g of Compound 2-21. (Yield: 73%, MS: [M+H]+=831)

### Preparation Example 2-22

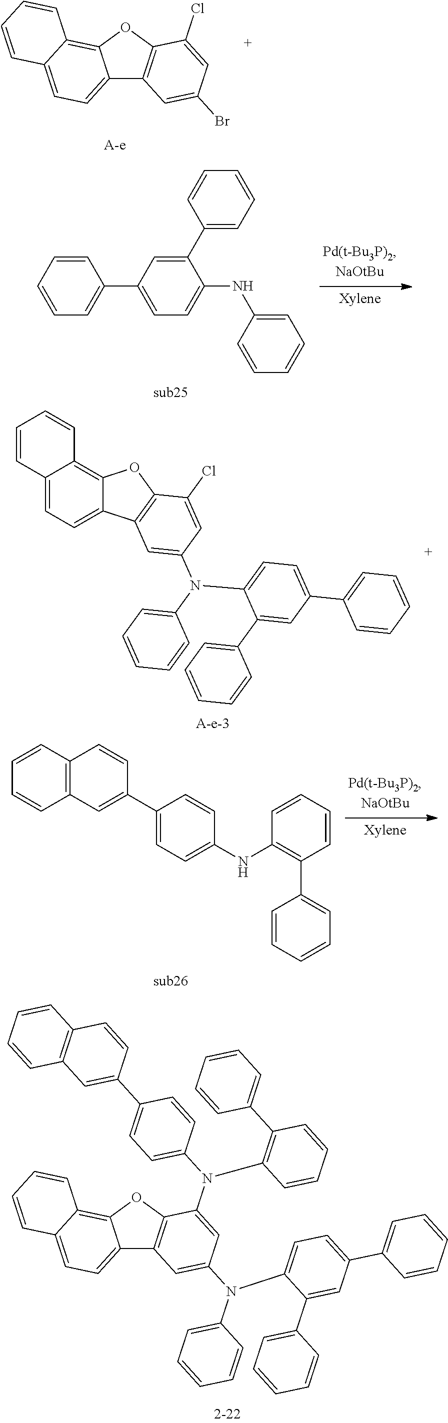 [SMILES: Clc1cc(Br)cc2c1oc1c3ccccc3ccc21 ; c1ccc(Nc2ccc(-c3ccccc3)cc2-c2ccccc2)cc1 ; Clc1cc(N(c2ccccc2)c2ccc(-c3ccccc3)cc2-c2ccccc2)cc2c1oc1c3ccccc3ccc21 ; c1ccc(-c2ccccc2Nc2ccc(-c3ccc4ccccc4c3)cc2)cc1 ; c1ccc(-c2ccc(N(c3ccccc3)c3cc(N(c4ccc(-c5ccc6ccccc6c5)cc4)c4ccccc4-c4ccccc4)c4oc5c6ccccc6ccc5c4c3)c(-c3ccccc3)c2)cc1]

Compound A-e (10 g, 30.2 mmol), sub25 (10.2 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.6 g of Compound A-e-3. (Yield: 73%, MS: [M+H]+=572)

Compound A-e-3 (10 g, 17.5 mmol), sub26 (6.8 g, 18.4 mmol) and sodium tert-butoxide (2.2 g, 22.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.7 g of Compound 2-22. (Yield: 74%, MS: [M+H]+=907)

### Preparation Example 2-23

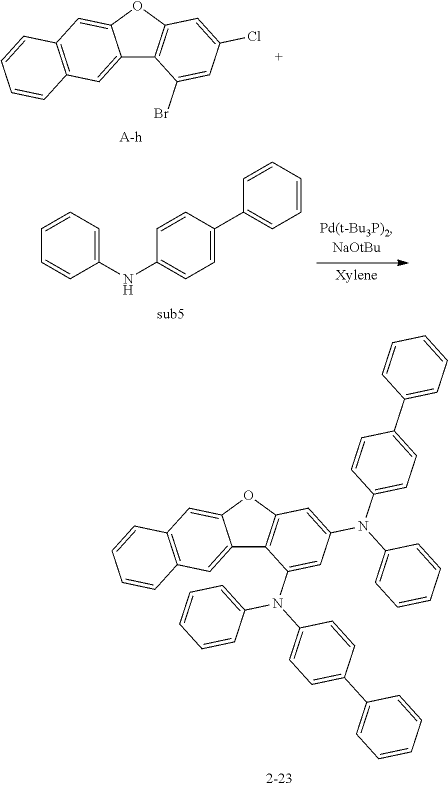 [SMILES: Clc1cc(Br)c2c(c1)oc1cc3ccccc3cc12 ; c1ccc(Nc2ccc(-c3ccccc3)cc2)cc1 ; c1ccc(-c2ccc(N(c3ccccc3)c3cc(N(c4ccccc4)c4ccc(-c5ccccc5)cc4)c4c(c3)oc3cc5ccccc5cc34)cc2)cc1]

Compound A-h (10 g, 30.2 mmol), sub5 (15.2 g, 61.8 mmol) and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11 g of Compound 2-23. (Yield: 52%, MS: [M+H]+=705)

### Preparation Example 2-24

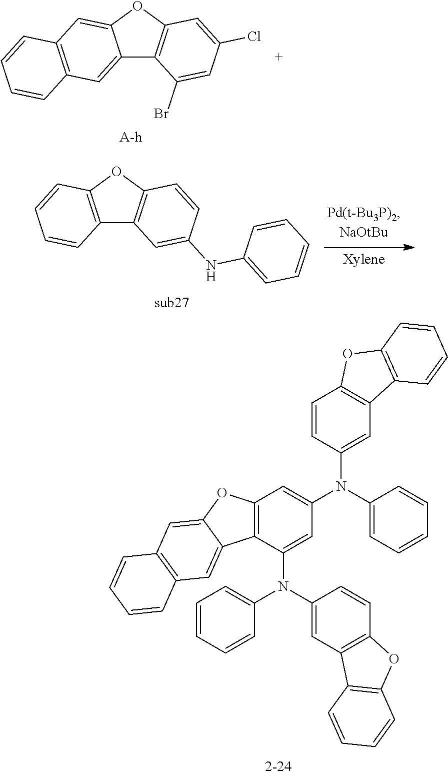 [SMILES: Clc1cc(Br)c2c(c1)oc1cc3ccccc3cc12 ; c1ccc(Nc2ccc3oc4ccccc4c3c2)cc1 ; c1ccc(N(c2cc(N(c3ccccc3)c3ccc4oc5ccccc5c4c3)c3c(c2)oc2cc4ccccc4cc23)c2ccc3oc4ccccc4c3c2)cc1]

Compound A-h (10 g, 30.2 mmol), sub27 (16 g, 61.8 mmol) and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.4 g of Compound 2-24. (Yield: 65%, MS: [M+H]+=733)

### Preparation Example 2-25

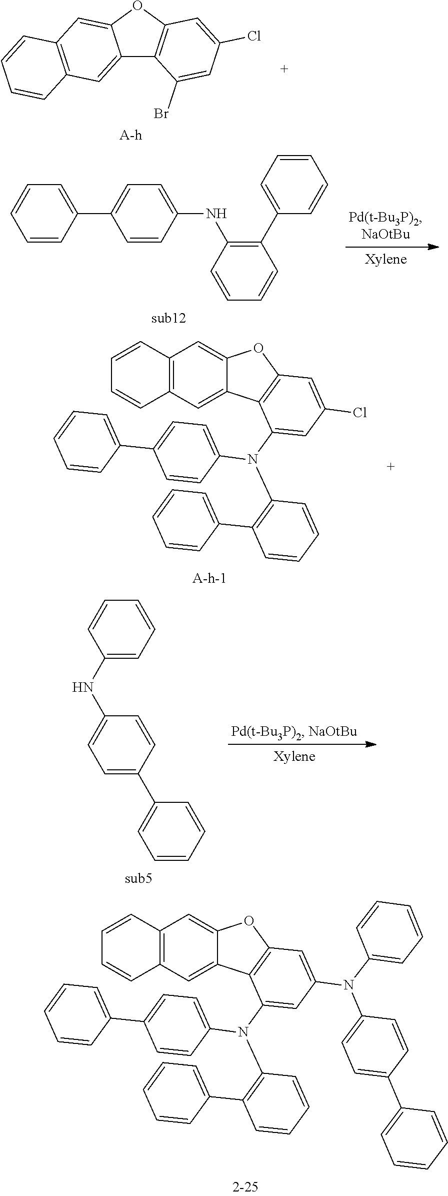 [SMILES: Clc1cc(Br)c2c(c1)oc1cc3ccccc3cc12 ; c1ccc(-c2ccc(Nc3ccccc3-c3ccccc3)cc2)cc1 ; Clc1cc(N(c2ccc(-c3ccccc3)cc2)c2ccccc2-c2ccccc2)c2c(c1)oc1cc3ccccc3cc12 ; c1ccc(Nc2ccc(-c3ccccc3)cc2)cc1 ; c1ccc(-c2ccc(N(c3ccccc3)c3cc(N(c4ccc(-c5ccccc5)cc4)c4ccccc4-c4ccccc4)c4c(c3)oc3cc5ccccc5cc34)cc2)cc1]

Compound A-h (10 g, 30.2 mmol), sub12 (10.2 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.4 g of Compound A-h-1. (Yield: 66%, MS: [M+H]+=572)

Compound A-h-1 (10 g, 17.5 mmol), sub5 (4.5 g, 18.4 mmol) and sodium tert-butoxide (2.2 g, 22.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.2 g of Compound 2-25. (Yield: 60%, MS: [M+H]+=781)

### Preparation Example 2-26

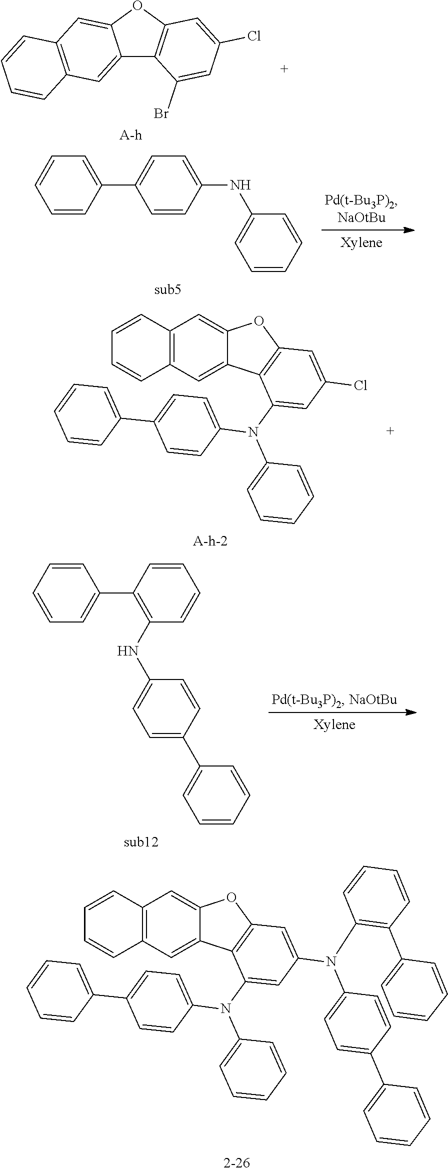

Compound A-h (10 g, 30.2 mmol), sub5 (7.8 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.9 g of Compound A-h-2. (Yield: 73%, MS: [M+H]+=496)

Compound A-h-2 (10 g, 20.2 mmol), sub12 (6.8 g, 21.2 mmol) and sodium tert-butoxide (2.5 g, 26.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.2 g of Compound 2-26. (Yield: 71%, MS: [M+H]+=781)

### Preparation Example 2-27

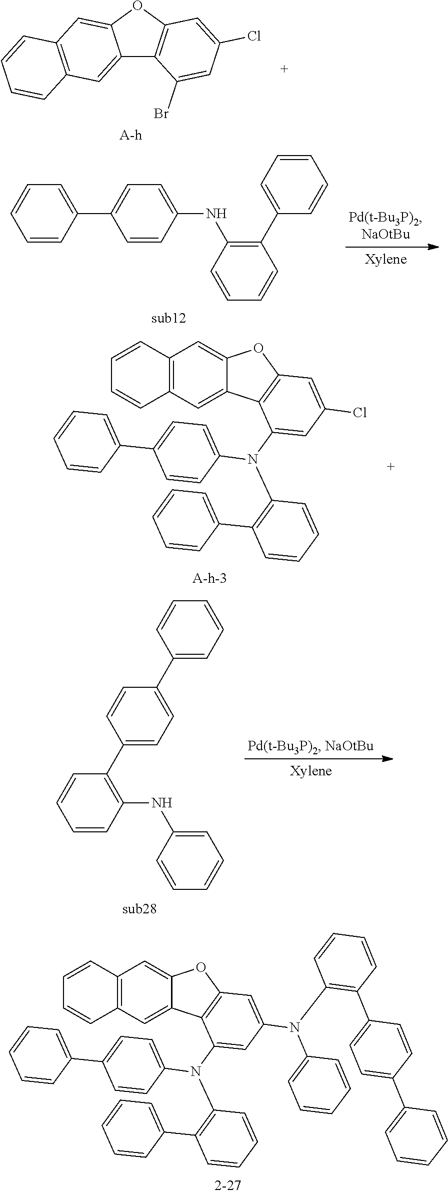 [SMILES: Clc1cc(Br)c2c(c1)oc1cc3ccccc3cc12 ; c1ccc(-c2ccc(Nc3ccccc3-c3ccccc3)cc2)cc1 ; Clc1cc(N(c2ccc(-c3ccccc3)cc2)c2ccccc2-c2ccccc2)c2c(c1)oc1cc3ccccc3cc12 ; c1ccc(Nc2ccccc2-c2ccc(-c3ccccc3)cc2)cc1 ; c1ccc(-c2ccc(-c3ccccc3N(c3ccccc3)c3cc(N(c4ccc(-c5ccccc5)cc4)c4ccccc4-c4ccccc4)c4c(c3)oc3cc5ccccc5cc34)cc2)cc1]

Compound A-h (10 g, 30.2 mmol), sub12 (10.2 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.7 g of Compound A-h-3. (Yield: 62%, MS: [M+H]+=572)

Compound A-h-3 (10 g, 17.5 mmol), sub28 (5.9 g, 18.4 mmol) and sodium tert-butoxide (2.2 g, 22.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.9 g of Compound 2-27. (Yield: 66%, MS: [M+H]+=857)

### Preparation Example 2-28

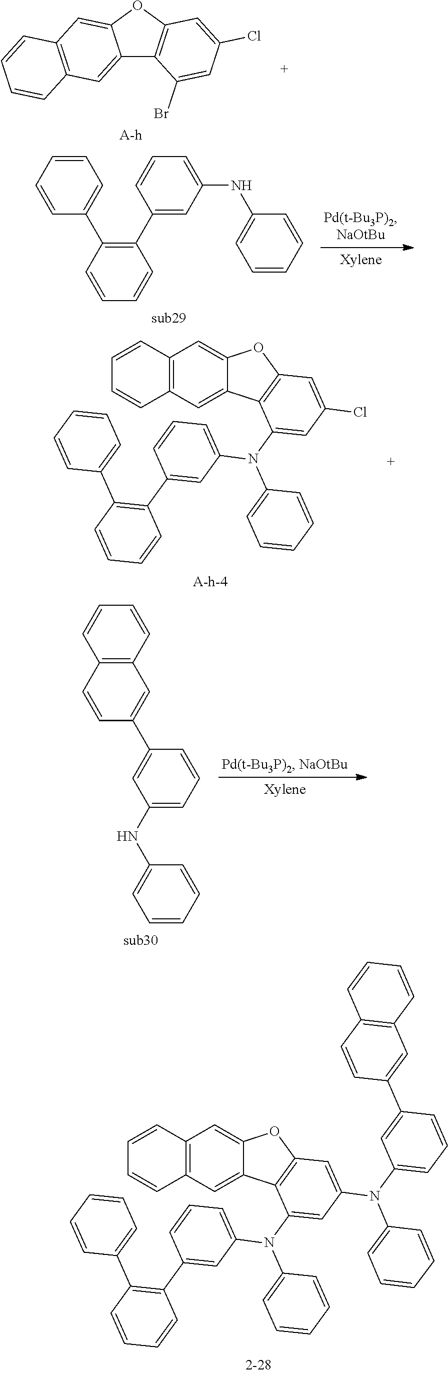

Compound A-h (10 g, 30.2 mmol), sub29 (10.2 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.9 g of Compound A-h-4. (Yield: 69%, MS: [M+H]+=572)

Compound A-h-4 (10 g, 17.5 mmol), sub30 (5.4 g, 18.4 mmol) and sodium tert-butoxide (2.2 g, 22.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.7 g of Compound 2-28. (Yield: 60%, MS: [M+H]+=831)

### Preparation Example 2-29

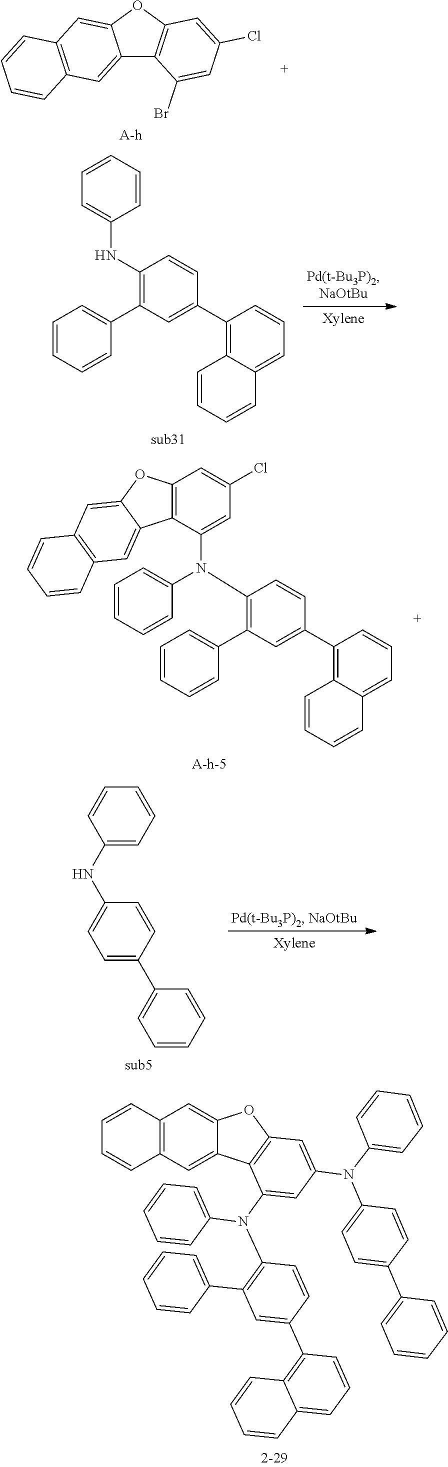 [SMILES: Clc1cc(Br)c2c(c1)oc1cc3ccccc3cc12 ; c1ccc(Nc2ccc(-c3cccc4ccccc34)cc2-c2ccccc2)cc1 ; Clc1cc(N(c2ccccc2)c2ccc(-c3cccc4ccccc34)cc2-c2ccccc2)c2c(c1)oc1cc3ccccc3cc12 ; c1ccc(Nc2ccc(-c3ccccc3)cc2)cc1 ; c1ccc(-c2ccc(N(c3ccccc3)c3cc(N(c4ccccc4)c4ccc(-c5cccc6ccccc56)cc4-c4ccccc4)c4c(c3)oc3cc5ccccc5cc34)cc2)cc1]

Compound A-h (10 g, 30.2 mmol), sub31 (11.8 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.4 g of Compound A-h-5. (Yield: 61%, MS: [M+H]+=622)

Compound A-h-5 (10 g, 16.1 mmol), sub5 (4.1 g, 16.9 mmol) and sodium tert-butoxide (2 g, 20.9 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.1 g of Compound 2-29. (Yield: 68%, MS: [M+H]+=831)

### Preparation Example 2-30

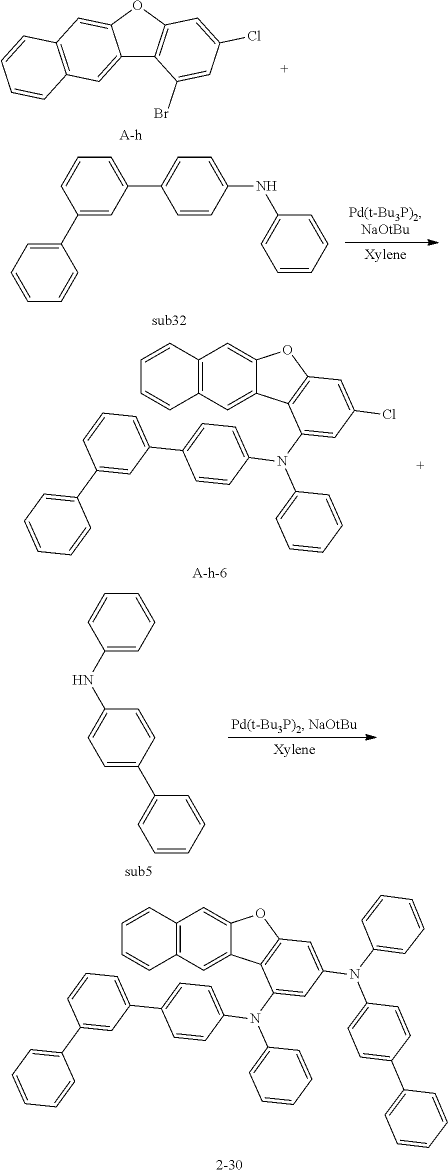

Compound A-h (10 g, 30.2 mmol), sub32 (10.2 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.7 g of Compound A-h-6. (Yield: 74%, MS: [M+H]+=572)

Compound A-h-6 (10 g, 17.5 mmol), sub5 (4.5 g, 18.4 mmol) and sodium tert-butoxide (2.2 g, 22.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.1 g of Compound 2-30. (Yield: 67%, MS: [M+H]+=781)

### Preparation Example 2-31

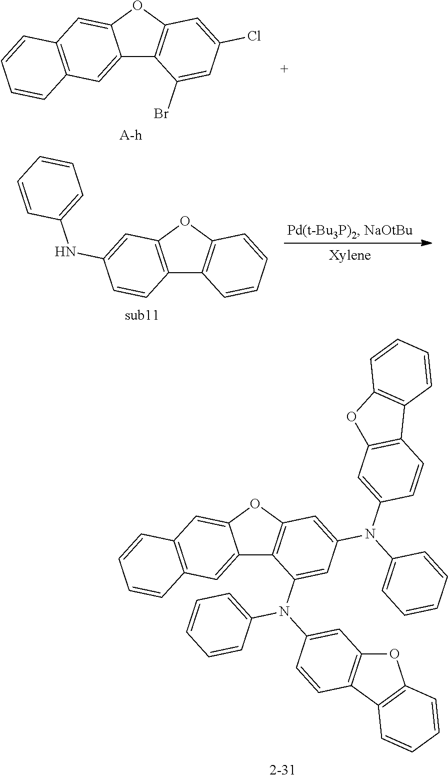 [SMILES: Clc1cc(Br)c2c(c1)oc1cc3ccccc3cc12 ; c1ccc(Nc2ccc3c(c2)oc2ccccc23)cc1 ; c1ccc(N(c2ccc3c(c2)oc2ccccc23)c2cc(N(c3ccccc3)c3ccc4c(c3)oc3ccccc34)c3c(c2)oc2cc4ccccc4cc23)cc1]

Compound A-h (10 g, 30.2 mmol), sub11 (15.7 g, 61.8 mmol) and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.1 g of Compound 2-31. (Yield: 73%, MS: [M+H]+=734)

### Preparation Example 2-32

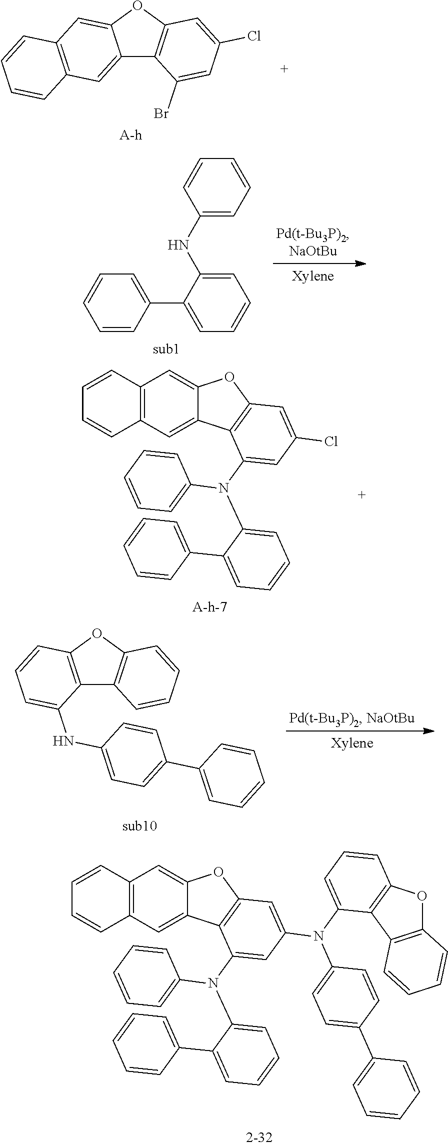

Compound A-h (10 g, 30.2 mmol), sub1 (7.8 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.2 g of Compound A-h-7. (Yield: 68%, MS: [M+H]+=496)

Compound A-h-7 (10 g, 20.2 mmol), sub10 (7.1 g, 21.2 mmol) and sodium tert-butoxide (2.5 g, 26.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.7 g of Compound 2-32. (Yield: 73%, MS: [M+H]+=795)

### Preparation Example 2-33

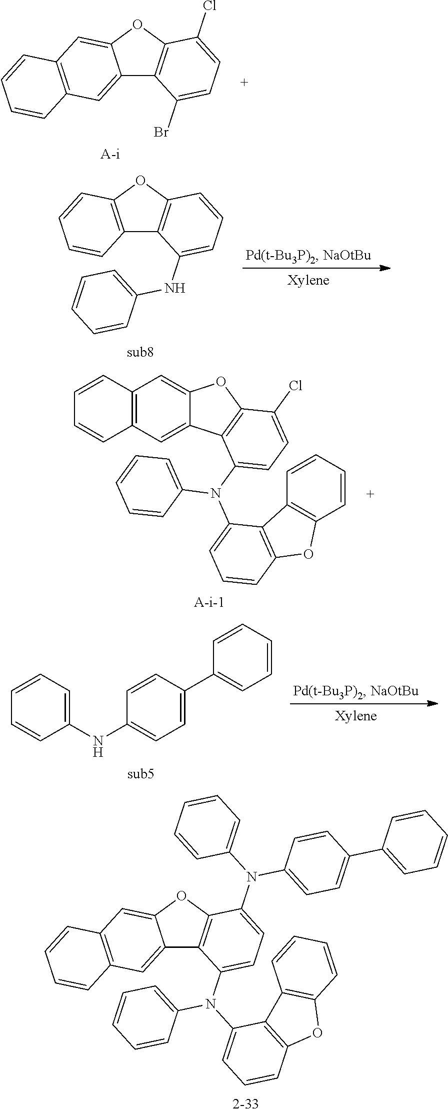

Compound A-i (10 g, 30.2 mmol), sub8 (7.9 g, 30.5 mmol) and sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.6 g of Compound A-i-1. (Yield: 56%, MS: [M+H]+=510)

Compound A-i-1 (10 g, 19.6 mmol), sub5 (4.9 g, 20 mmol) and sodium tert-butoxide (2.4 g, 25.5 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.2 g of Compound 2-33. (Yield: 65%, MS: [M+H]+=719)

### Preparation Example 2-34

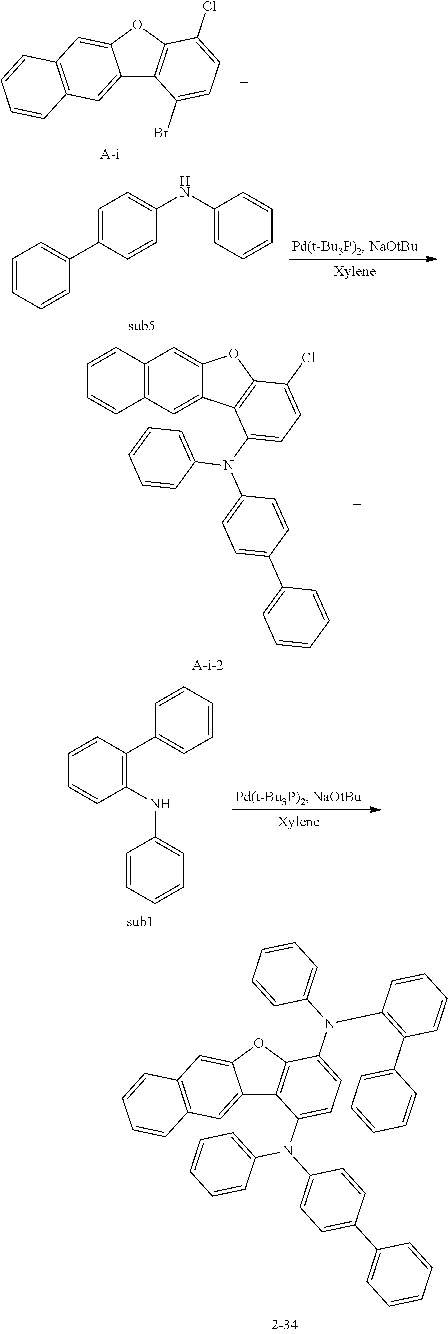 [SMILES: Clc1ccc(Br)c2c1oc1cc3ccccc3cc12 ; c1ccc(Nc2ccc(-c3ccccc3)cc2)cc1 ; Clc1ccc(N(c2ccccc2)c2ccc(-c3ccccc3)cc2)c2c1oc1cc3ccccc3cc12 ; c1ccc(Nc2ccccc2-c2ccccc2)cc1 ; c1ccc(-c2ccc(N(c3ccccc3)c3ccc(N(c4ccccc4)c4ccccc4-c4ccccc4)c4oc5cc6ccccc6cc5c34)cc2)cc1]

Compound A-i (10 g, 30.2 mmol), sub5 (7.5 g, 30.5 mmol) and sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.8 g of Compound A-i-2. (Yield: 59%, MS: [M+H]+=496)

Compound A-i-2 (10 g, 20.2 mmol), sub1 (5 g, 20.6 mmol) and sodium tert-butoxide (2.5 g, 26.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.5 g of Compound 2-34. (Yield: 53%, MS: [M+H]+=705)

### Preparation Example 2-35

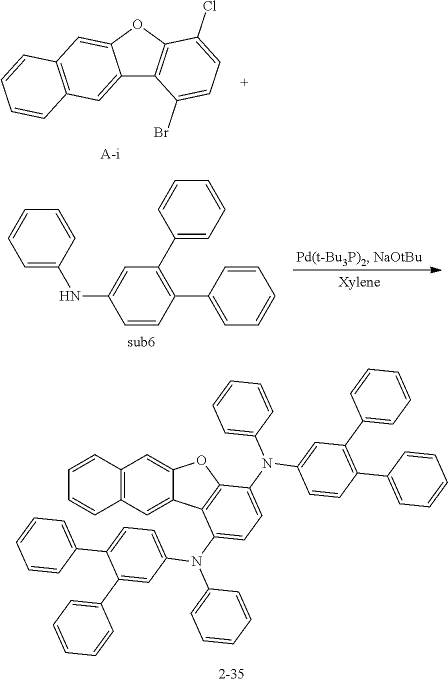 [SMILES: Clc1ccc(Br)c2c1oc1cc3ccccc3cc12 ; c1ccc(Nc2ccc(-c3ccccc3)c(-c3ccccc3)c2)cc1 ; c1ccc(-c2ccc(N(c3ccccc3)c3ccc(N(c4ccccc4)c4ccc(-c5ccccc5)c(-c5ccccc5)c4)c4c3oc3cc5ccccc5cc34)cc2-c2ccccc2)cc1]

Compound A-i (10 g, 30.2 mmol), sub6 (19.9 g, 61.8 mmol) and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 17.3 g of Compound 2-35. (Yield: 67%, MS: [M+H]+=858)

### Preparation Example 2-36

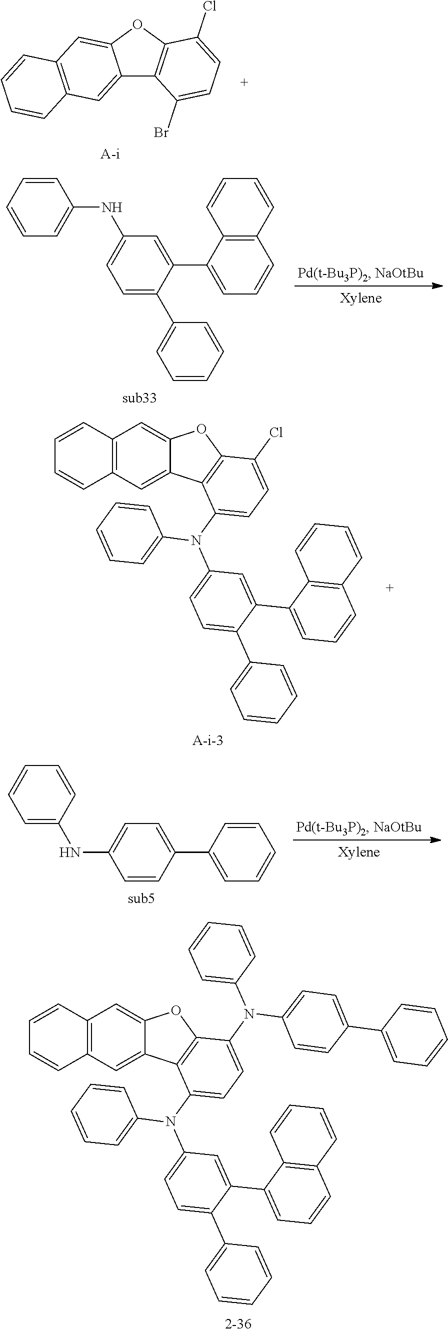

Compound A-i (10 g, 30.2 mmol), sub33 (11.8 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.7 g of Compound A-i-3. (Yield: 68%, MS: [M+H]+=622)

Compound A-i-3 (10 g, 16.1 mmol), sub5 (4.1 g, 16.9 mmol) and sodium tert-butoxide (2 g, 20.9 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.3 g of Compound 2-36. (Yield: 62%, MS: [M+H]+=831)

### Preparation Example 2-37

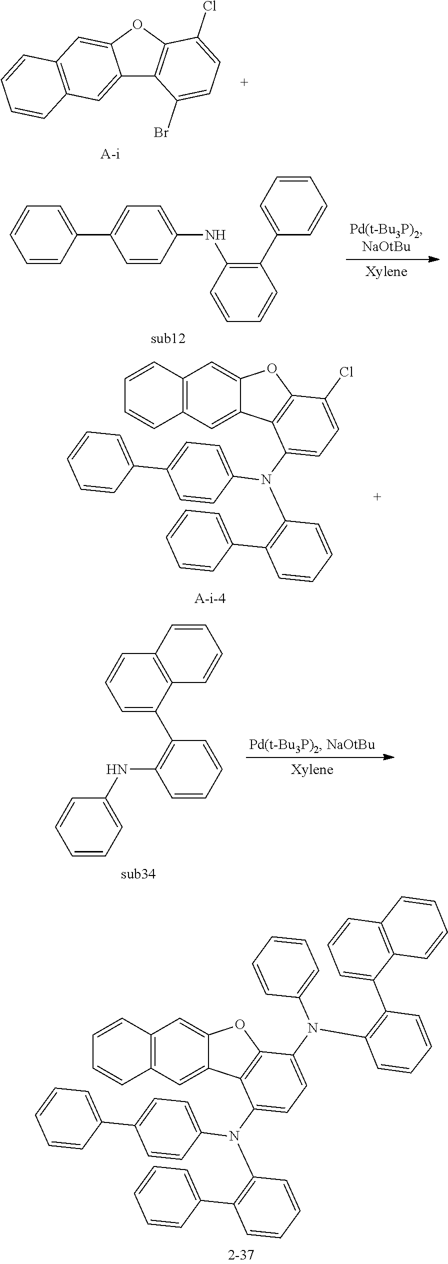

Compound A-i (10 g, 30.2 mmol), sub12 (10.2 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.7 g of Compound A-i-4. (Yield: 62%, MS: [M+H]+=572)

Compound A-i-4 (10 g, 17.5 mmol), sub34 (5.4 g, 18.4 mmol) and sodium tert-butoxide (2.2 g, 22.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9 g of Compound 2-37. (Yield: 62%, MS: [M+H]+=831)

### Preparation Example 2-38

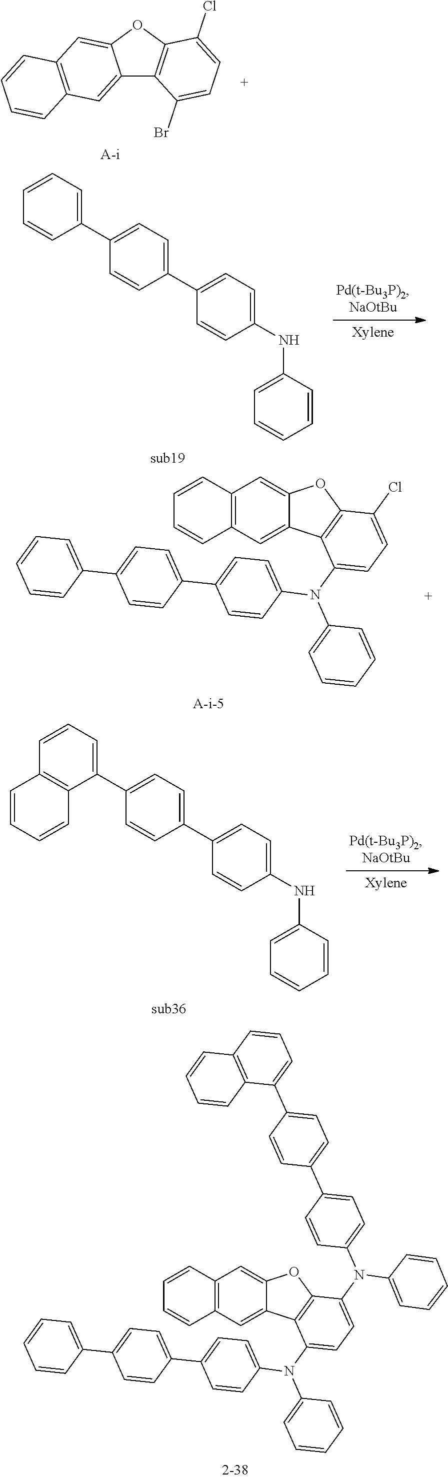

Compound A-i (10 g, 30.2 mmol), sub19 (10.2 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.4 g of Compound A-i-5. (Yield: 72%, MS: [M+H]+=572)

Compound A-i-5 (10 g, 17.5 mmol), sub35 (6.8 g, 18.4 mmol) and sodium tert-butoxide (2.2 g, 22.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.9 g of Compound 2-38. (Yield: 75%, MS: [M+H]+=907)

### Preparation Example 2-39

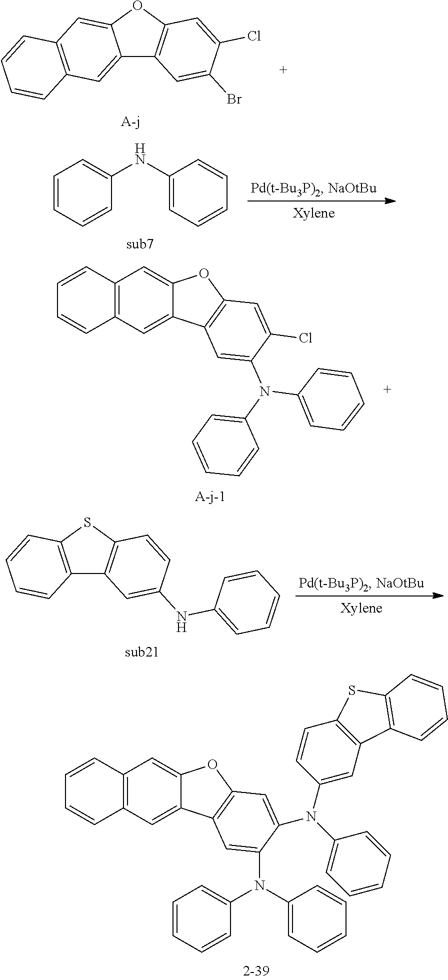

Compound A-j (10 g, 30.2 mmol), sub7 (5.2 g, 30.5 mmol) and sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.2 g of Compound A-j-1. (Yield: 65%, MS: [M+H]+=420)

Compound A-j-1 (10 g, 23.8 mmol), sub21 (6.7 g, 24.3 mmol) and sodium tert-butoxide (3 g, 31 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.2 g of Compound 2-39. (Yield: 65%, MS: [M+H]+=659)

### Preparation Example 2-40

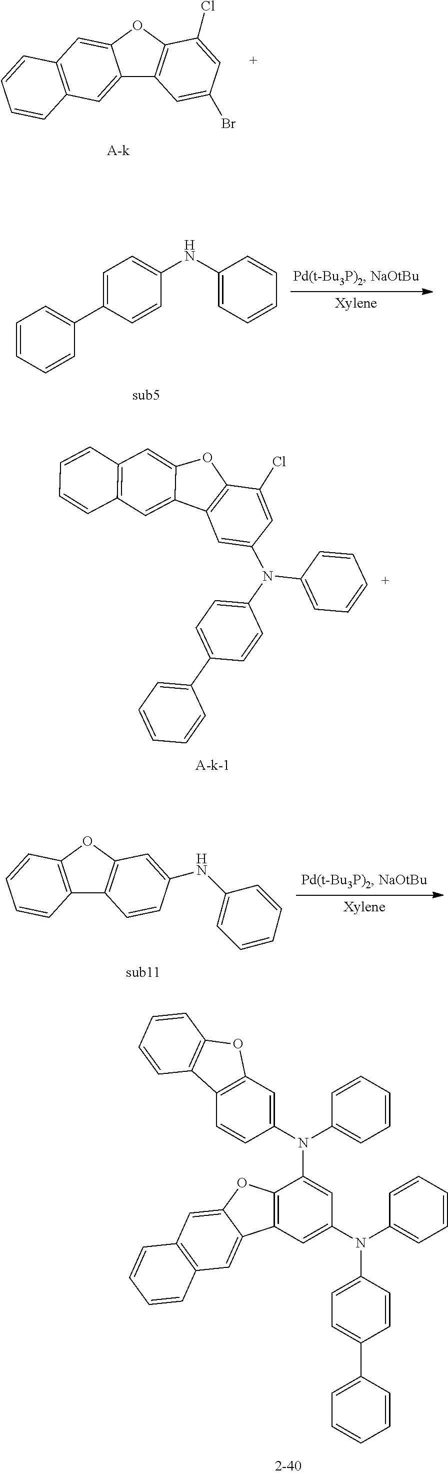 [SMILES: Clc1cc(Br)cc2c1oc1cc3ccccc3cc12 ; c1ccc(Nc2ccc(-c3ccccc3)cc2)cc1 ; Clc1cc(N(c2ccccc2)c2ccc(-c3ccccc3)cc2)cc2c1oc1cc3ccccc3cc12 ; c1ccc(Nc2ccc3c(c2)oc2ccccc23)cc1 ; c1ccc(-c2ccc(N(c3ccccc3)c3cc(N(c4ccccc4)c4ccc5c(c4)oc4ccccc45)c4oc5cc6ccccc6cc5c4c3)cc2)cc1]

Compound A-k (10 g, 30.2 mmol), sub5 (7.5 g, 30.5 mmol) and sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added 20 thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.7 g of Compound A-k-1. (Yield: 65%, MS: [M+H]+=496)

Compound A-k-1 (10 g, 20.2 mmol), sub11 (5.3 g, 20.6 mmol) and sodium tert-butoxide (2.5 g, 26.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.4 g of Compound 2-40. (Yield: 65%, MS: [M+H]+=719)

### Preparation Example 2-41

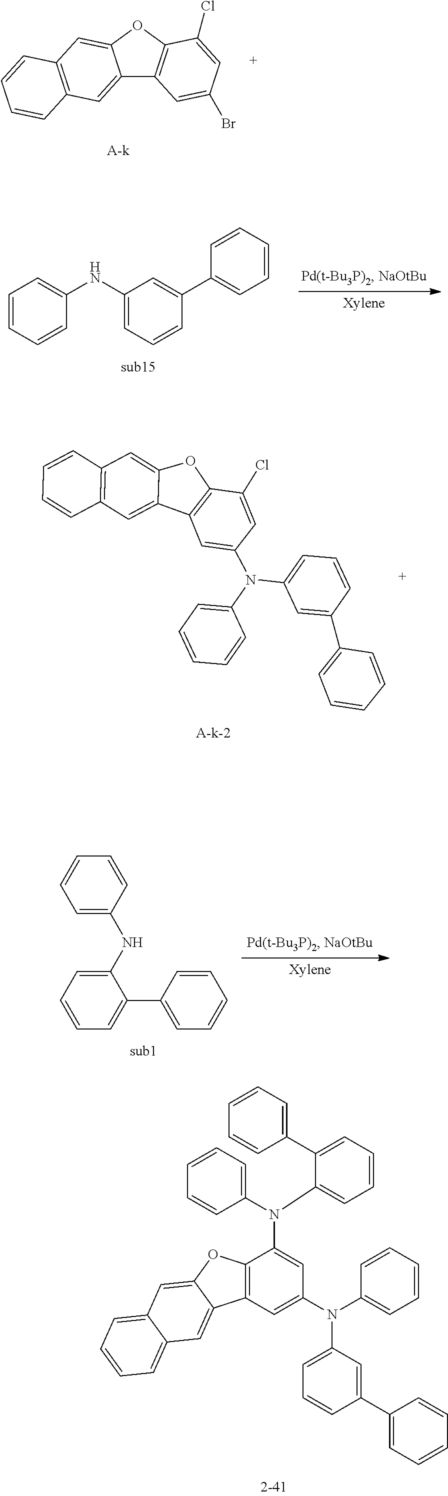 [SMILES: Clc1cc(Br)cc2c1oc1cc3ccccc3cc12 ; c1ccc(Nc2cccc(-c3ccccc3)c2)cc1 ; Clc1cc(N(c2ccccc2)c2cccc(-c3ccccc3)c2)cc2c1oc1cc3ccccc3cc12 ; c1ccc(Nc2ccccc2-c2ccccc2)cc1 ; c1ccc(-c2cccc(N(c3ccccc3)c3cc(N(c4ccccc4)c4ccccc4-c4ccccc4)c4oc5cc6ccccc6cc5c4c3)c2)cc1]

Compound A-k (10 g, 30.2 mmol), sub15 (7.5 g, 30.5 mmol) and sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.7 g of Compound A-k-2. (Yield: 58%, MS: [M+H]+=496)

Compound A-k-2 (10 g, 20.2 mmol), sub1 (5 g, 20.6 mmol) and sodium tert-butoxide (2.5 g, 26.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.7 g of Compound 2-41. (Yield: 54%, MS: [M+H]+=705)

### Preparation Example 2-42

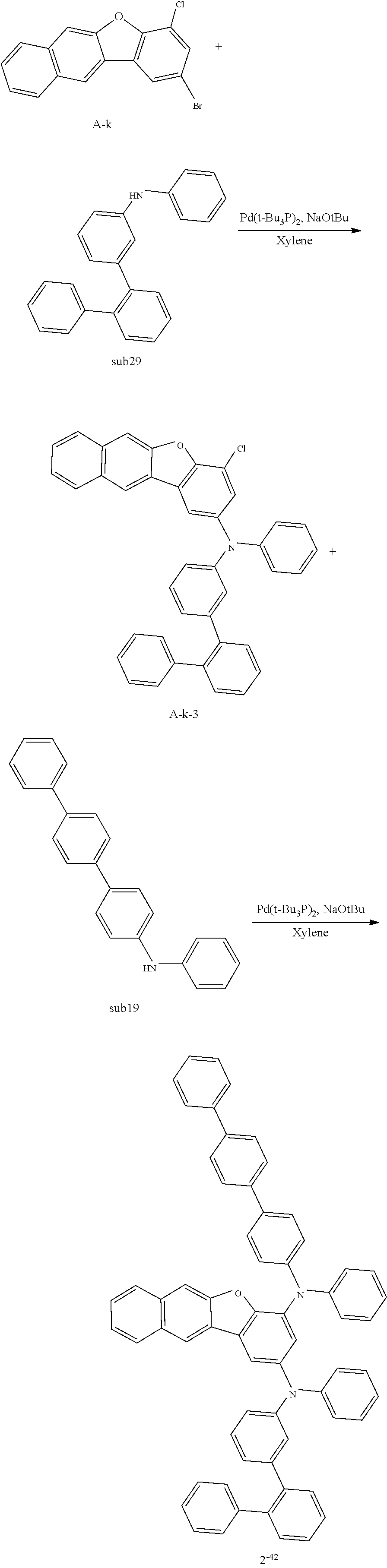 [SMILES: Clc1cc(Br)cc2c1oc1cc3ccccc3cc12 ; c1ccc(Nc2cccc(-c3ccccc3-c3ccccc3)c2)cc1 ; Clc1cc(N(c2ccccc2)c2cccc(-c3ccccc3-c3ccccc3)c2)cc2c1oc1cc3ccccc3cc12 ; c1ccc(Nc2ccc(-c3ccc(-c4ccccc4)cc3)cc2)cc1 ; c1ccc(-c2ccc(-c3ccc(N(c4ccccc4)c4cc(N(c5ccccc5)c5cccc(-c6ccccc6-c6ccccc6)c5)cc5c4oc4cc6ccccc6cc45)cc3)cc2)cc1]

Compound A-k (10 g, 30.2 mmol), sub29 (10.2 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.3 g of Compound A-k-3. (Yield: 60%, MS: [M+H]+=572)

Compound A-k-3 (10 g, 17.5 mmol), sub19 (5.9 g, 18.4 mmol) and sodium tert-butoxide (2.2 g, 22.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.1 g of Compound 2-42. (Yield: 74%, MS: [M+H]+=857)

### Preparation Example 2-43

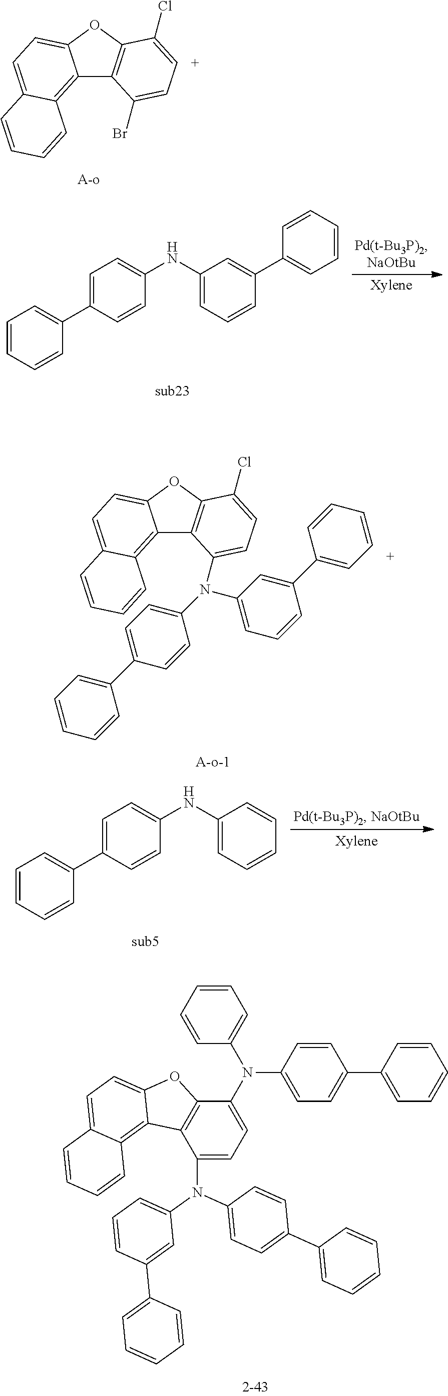 [SMILES: Clc1ccc(Br)c2c1oc1ccc3ccccc3c12 ; c1ccc(-c2ccc(Nc3cccc(-c4ccccc4)c3)cc2)cc1 ; Clc1ccc(N(c2ccc(-c3ccccc3)cc2)c2cccc(-c3ccccc3)c2)c2c1oc1ccc3ccccc3c12 ; c1ccc(Nc2ccc(-c3ccccc3)cc2)cc1 ; c1ccc(-c2ccc(N(c3ccccc3)c3ccc(N(c4ccc(-c5ccccc5)cc4)c4cccc(-c5ccccc5)c4)c4c3oc3ccc5ccccc5c34)cc2)cc1]

Compound A-o (10 g, 30.2 mmol), sub23 (9.8 g, 30.5 mmol) and sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.5 g of Compound A-o-1. (Yield: 61%, MS: [M+H]+=572)

Compound A-o-1 (10 g, 17.5 mmol), sub5 (4.4 g, 17.8 mmol) and sodium tert-butoxide (2.2 g, 22.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.6 g of Compound 2-43. (Yield: 63%, MS: [M+H]+=781)

### Preparation Example 2-44

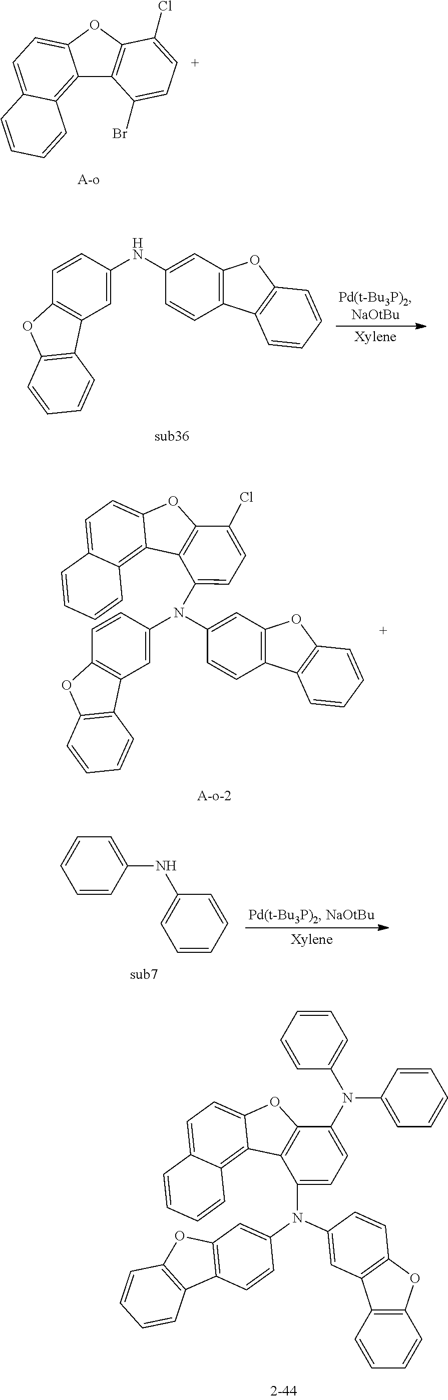 [SMILES: Clc1ccc(Br)c2c1oc1ccc3ccccc3c12 ; c1ccc2c(c1)oc1cc(Nc3ccc4oc5ccccc5c4c3)ccc12 ; Clc1ccc(N(c2ccc3c(c2)oc2ccccc23)c2ccc3oc4ccccc4c3c2)c2c1oc1ccc3ccccc3c12 ; c1ccc(Nc2ccccc2)cc1 ; c1ccc(N(c2ccccc2)c2ccc(N(c3ccc4c(c3)oc3ccccc34)c3ccc4oc5ccccc5c4c3)c3c2oc2ccc4ccccc4c23)cc1]

Compound A-o (10 g, 30.2 mmol), sub36 (10.6 g, 30.5 mmol) and sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 11.4 g of Compound A-o-2. (Yield: 63%, MS: [M+H]+=600)

Compound A-o-2 (10 g, 16.7 mmol), sub7 (2.9 g, 17 mmol) and sodium tert-butoxide (2.1 g, 21.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 6.3 g of Compound 2-44. (Yield: 52%, MS: [M+H]+=733)

### Preparation Example 2-45

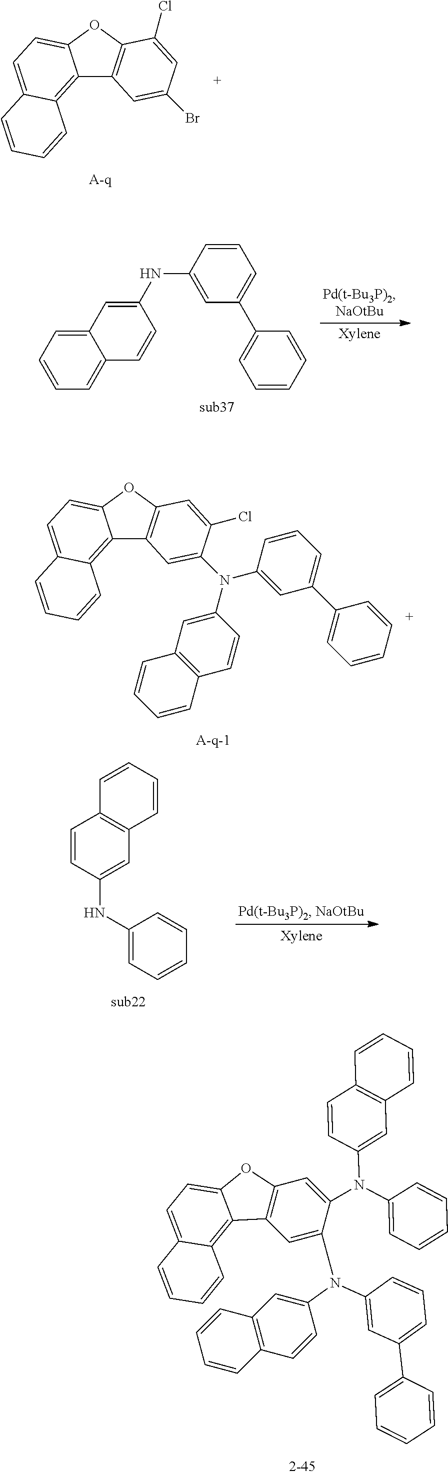 [SMILES: Clc1cc(Br)cc2c1oc1ccc3ccccc3c12 ; c1ccc(-c2cccc(Nc3ccc4ccccc4c3)c2)cc1 ; Clc1cc2oc3ccc4ccccc4c3c2cc1N(c1cccc(-c2ccccc2)c1)c1ccc2ccccc2c1 ; c1ccc(Nc2ccc3ccccc3c2)cc1 ; c1ccc(-c2cccc(N(c3ccc4ccccc4c3)c3cc4c(cc3N(c3ccccc3)c3ccc5ccccc5c3)oc3ccc5ccccc5c34)c2)cc1]

Compound A-q (10 g, 30.2 mmol), sub37 (13.1 g, 33.2 mmol) and sodium tert-butoxide (19.2 g, 90.5 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.9 g of Compound A-q-1. (Yield: 54%, MS: [M+H]+=546)

Compound A-q-1 (10 g, 18.3 mmol), sub22 (4.1 g, 18.7 mmol) and sodium tert-butoxide (2.3 g, 23.8 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.3 g of Compound 2-45. (Yield: 70%, MS: [M+H]+=729)

### Preparation Example 2-46

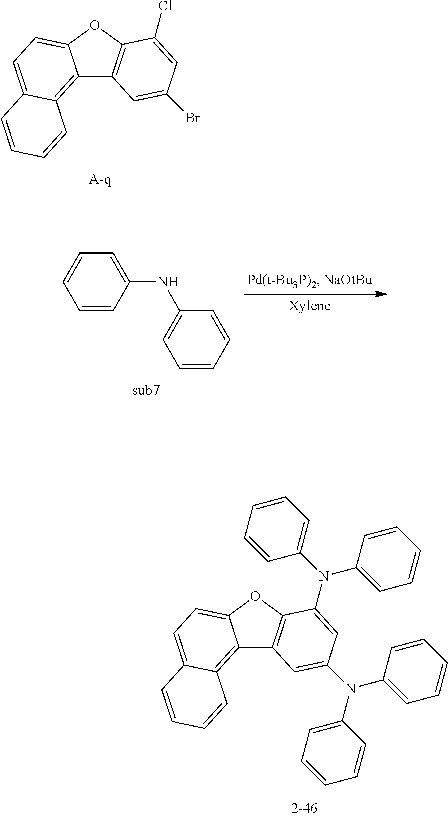 [SMILES: Clc1cc(Br)cc2c1oc1ccc3ccccc3c12 ; c1ccc(Nc2ccccc2)cc1 ; c1ccc(N(c2ccccc2)c2cc(N(c3ccccc3)c3ccccc3)c3oc4ccc5ccccc5c4c3c2)cc1]

Compound A-q (10 g, 30.2 mmol), sub7 (10.5 g, 61.8 mmol) and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.3 g of Compound 2-46. (Yield: 56%, MS: [M+H]+=553)

### Preparation Example 2-47

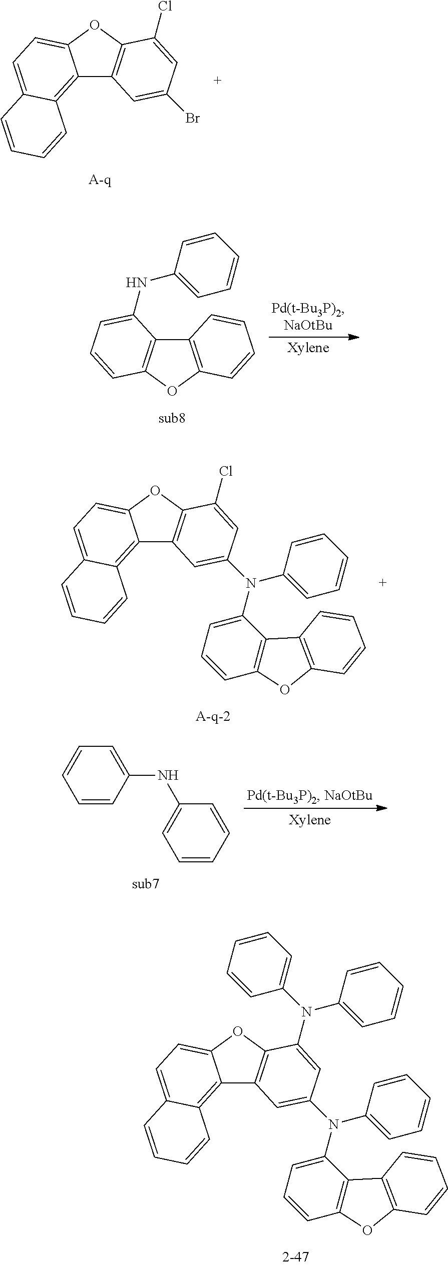 [SMILES: Clc1cc(Br)cc2c1oc1ccc3ccccc3c12 ; c1ccc(Nc2cccc3oc4ccccc4c23)cc1 ; Clc1cc(N(c2ccccc2)c2cccc3oc4ccccc4c23)cc2c1oc1ccc3ccccc3c12 ; c1ccc(Nc2ccccc2)cc1 ; c1ccc(N(c2ccccc2)c2cc(N(c3ccccc3)c3cccc4oc5ccccc5c34)cc3c2oc2ccc4ccccc4c23)cc1]

Compound A-q (10 g, 30.2 mmol), sub8 (7.9 g, 30.5 mmol) and sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9.8 g of Compound A-q-2. (Yield: 64%, MS: [M+H]+=510)

Compound A-q-2 (10 g, 19.6 mmol), sub7 (3.4 g, 20 mmol) and sodium tert-butoxide (2.4 g, 25.5 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 7.9 g of Compound 2-47. (Yield: 63%, MS: [M+H]+=643)

### Preparation Example 2-48

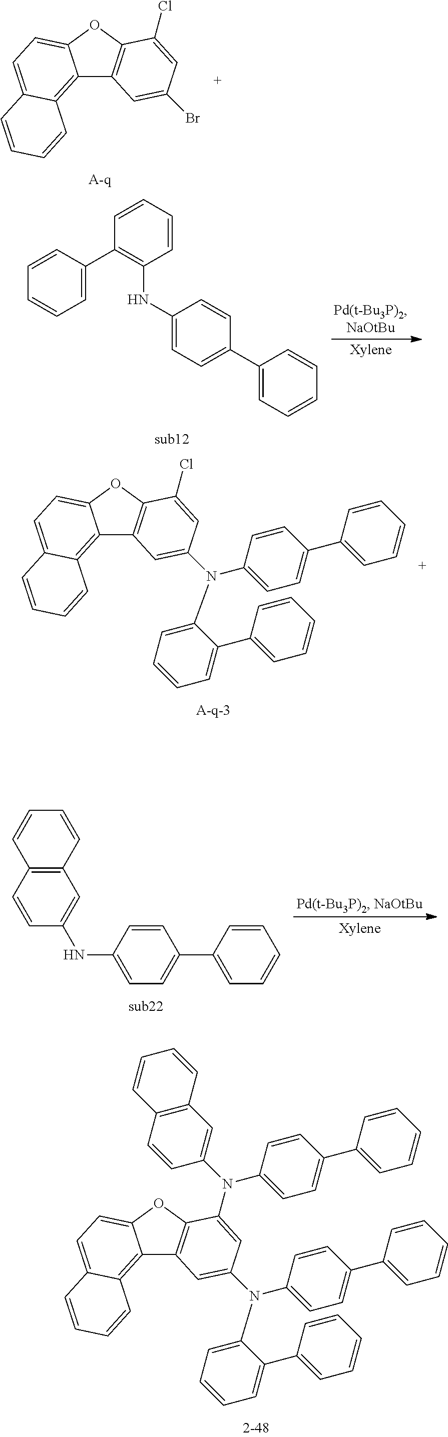 [SMILES: Clc1cc(Br)cc2c1oc1ccc3ccccc3c12 ; c1ccc(-c2ccc(Nc3ccccc3-c3ccccc3)cc2)cc1 ; Clc1cc(N(c2ccc(-c3ccccc3)cc2)c2ccccc2-c2ccccc2)cc2c1oc1ccc3ccccc3c12 ; c1ccc(-c2ccc(Nc3ccc4ccccc4c3)cc2)cc1 ; c1ccc(-c2ccc(N(c3cc(N(c4ccc(-c5ccccc5)cc4)c4ccc5ccccc5c4)c4oc5ccc6ccccc6c5c4c3)c3ccccc3-c3ccccc3)cc2)cc1]

Compound A-q (10 g, 30.2 mmol), sub12 (10.2 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.6 g of Compound A-q-3. (Yield: 73%, MS: [M+H]+=572)

Compound A-q-3 (10 g, 17.5 mmol), sub22 (5.4 g, 18.4 mmol) and sodium tert-butoxide (2.2 g, 22.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10.2 g of Compound 2-48. (Yield: 70%, MS: [M+H]+=831)

### Preparation Example 2-49

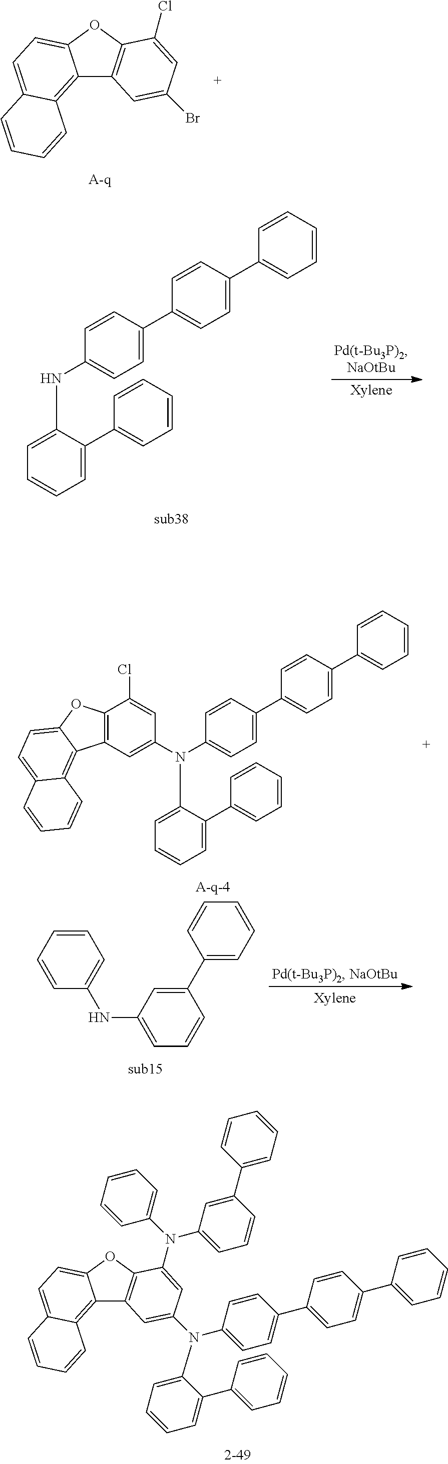 [SMILES: Clc1cc(Br)cc2c1oc1ccc3ccccc3c12 ; c1ccc(-c2ccc(-c3ccc(Nc4ccccc4-c4ccccc4)cc3)cc2)cc1 ; Clc1cc(N(c2ccc(-c3ccc(-c4ccccc4)cc3)cc2)c2ccccc2-c2ccccc2)cc2c1oc1ccc3ccccc3c12 ; c1ccc(Nc2cccc(-c3ccccc3)c2)cc1 ; c1ccc(-c2ccc(-c3ccc(N(c4cc(N(c5ccccc5)c5cccc(-c6ccccc6)c5)c5oc6ccc7ccccc7c6c5c4)c4ccccc4-c4ccccc4)cc3)cc2)cc1]

Compound A-q (10 g, 30.2 mmol), sub38 (12.6 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.6 g of Compound A-q-4. (Yield: 75%, MS: [M+H]+=648)

Compound A-q-4 (10 g, 15.4 mmol), sub15 (4 g, 16.2 mmol) and sodium tert-butoxide (1.9 g, 20.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 8.2 g of Compound 2-49. (Yield: 62%, MS: [M+H]+=857)

### Preparation Example 2-50

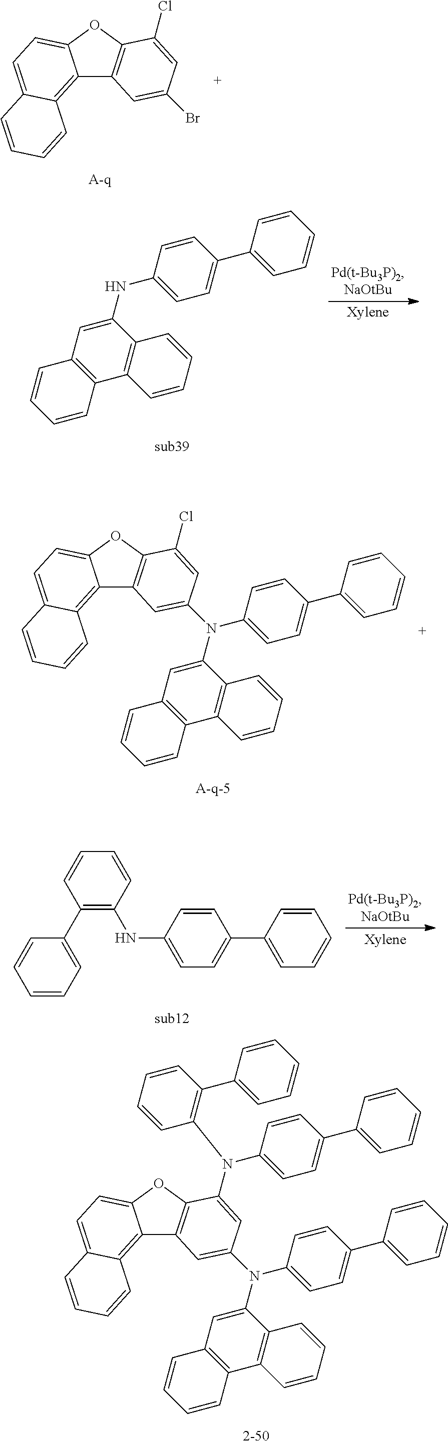 [SMILES: Clc1cc(Br)cc2c1oc1ccc3ccccc3c12 ; c1ccc(-c2ccc(Nc3cc4ccccc4c4ccccc34)cc2)cc1 ; Clc1cc(N(c2ccc(-c3ccccc3)cc2)c2cc3ccccc3c3ccccc23)cc2c1oc1ccc3ccccc3c12 ; c1ccc(-c2ccc(Nc3ccccc3-c3ccccc3)cc2)cc1 ; c1ccc(-c2ccc(N(c3cc(N(c4ccc(-c5ccccc5)cc4)c4ccccc4-c4ccccc4)c4oc5ccc6ccccc6c5c4c3)c3cc4ccccc4c4ccccc34)cc2)cc1]

Compound A-q (10 g, 30.2 mmol), sub39 (10.9 g, 31.7 mmol) and sodium tert-butoxide (3.8 g, 39.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 12.7 g of Compound A-q-5. (Yield: 71%, MS: [M+H]+=596)

Compound A-q-5 (10 g, 16.8 mmol), sub12 (5.7 g, 17.6 mmol) and sodium tert-butoxide (2.1 g, 21.8 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 9 g of Compound 2-50. (Yield: 61%, MS: [M+H]+=881)

### Preparation Example 2-51

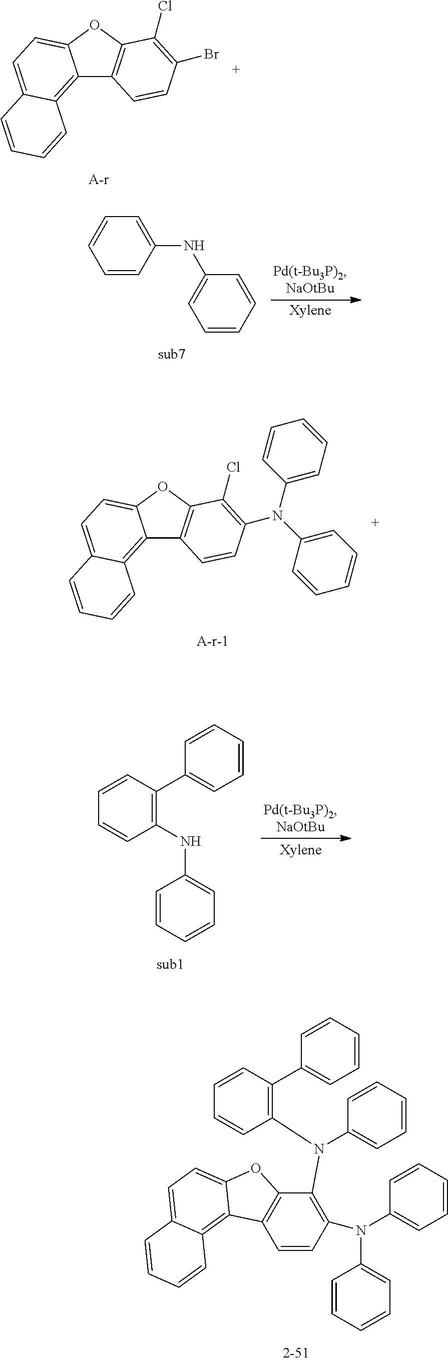 [SMILES: Clc1c(Br)ccc2c1oc1ccc3ccccc3c12 ; c1ccc(Nc2ccccc2)cc1 ; Clc1c(N(c2ccccc2)c2ccccc2)ccc2c1oc1ccc3ccccc3c12 ; c1ccc(Nc2ccccc2-c2ccccc2)cc1 ; c1ccc(-c2ccccc2N(c2ccccc2)c2c(N(c3ccccc3)c3ccccc3)ccc3c2oc2ccc4ccccc4c23)cc1]

Compound A-r (10 g, 30.2 mmol), sub7 (5.2 g, 30.5 mmol) and sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 6.6 g of Compound A-r-1. (Yield: 52%, MS: [M+H]+=420)

Compound A-r-1 (10 g, 23.8 mmol), sub1 (6 g, 24.3 mmol) and sodium tert-butoxide (3 g, 31 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 10 g of Compound 2-51. (Yield: 67%, MS: [M+H]+=629)

### Preparation Example 2-52

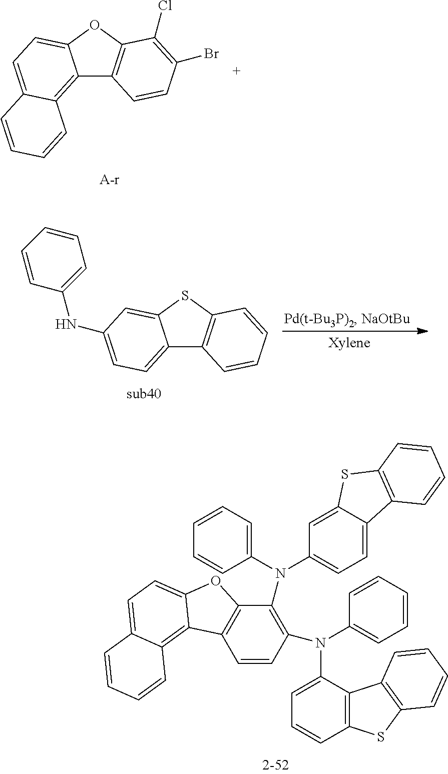 [SMILES: Clc1c(Br)ccc2c1oc1ccc3ccccc3c12 ; c1ccc(Nc2ccc3c(c2)sc2ccccc23)cc1 ; c1ccc(N(c2ccc3c(c2)sc2ccccc23)c2c(N(c3ccccc3)c3cccc4sc5ccccc5c34)ccc3c2oc2ccc4ccccc4c23)cc1]

Compound A-r (10 g, 30.2 mmol), sub40 (17 g, 61.8 mmol) and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. When the reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.9 g of Compound 2-52. (Yield: 69%, MS: [M+H]+=765)

### EXAMPLES

### Example 1

A glass substrate on which a thin film of ITO (indium tin oxide) was coated in a thickness of 1,000 Å was put into distilled water containing the detergent dissolved therein and washed by the ultrasonic wave. In this case, the used detergent was a product commercially available from Fisher Co. and the distilled water was one which had been twice filtered by using a filter commercially available from Millipore Co. The ITO was washed for 30 minutes, and ultrasonic washing was then repeated twice for 10 minutes by using distilled water. After the washing with distilled water was completed, the substrate was ultrasonically washed with isopropyl alcohol, acetone, and methanol solvent, and dried, after which it was transported to a plasma cleaner. Then, the substrate was cleaned with oxygen plasma for 5 minutes, and then transferred to a vacuum evaporator.

On the ITO transparent electrode thus prepared, the following compound HI-1 was formed in a thickness of 1150 Å as a hole injection layer, but the following compound A-1 was p-doped at a concentration of 1.5 wt. %. The following compound HT-1 was vacuum deposited on the hole injection layer to form a hole transport layer with a film thickness of 800 Å. Then, the following compound EB-1 was vacuum deposited on the hole transport layer to a film thickness of 150 Å to form an electron blocking layer. Then, Compound 1-1, Compound 2-1 and Compound Dp-7 prepared in Preparation Examples were vacuum deposited in a weight ratio of 49:49:2 on the EB-1 deposited film to form a red light emitting layer with a film thickness of 400 Å. The following compound HB-1 was vacuum deposited on the light emitting layer to a film thickness of 30 Å to form a hole blocking layer. The following compound ET-1 and the following compound LiQ were vacuum deposited in a weight ratio of 2:1 on the hole blocking layer to form an electron injection and transport layer with a film thickness of 300 Å. Lithium fluoride (LiF) and aluminum were sequentially deposited to have a thickness of 12 Å and 1,000 Å, respectively, on the electron injection and transport layer, thereby forming a cathode.

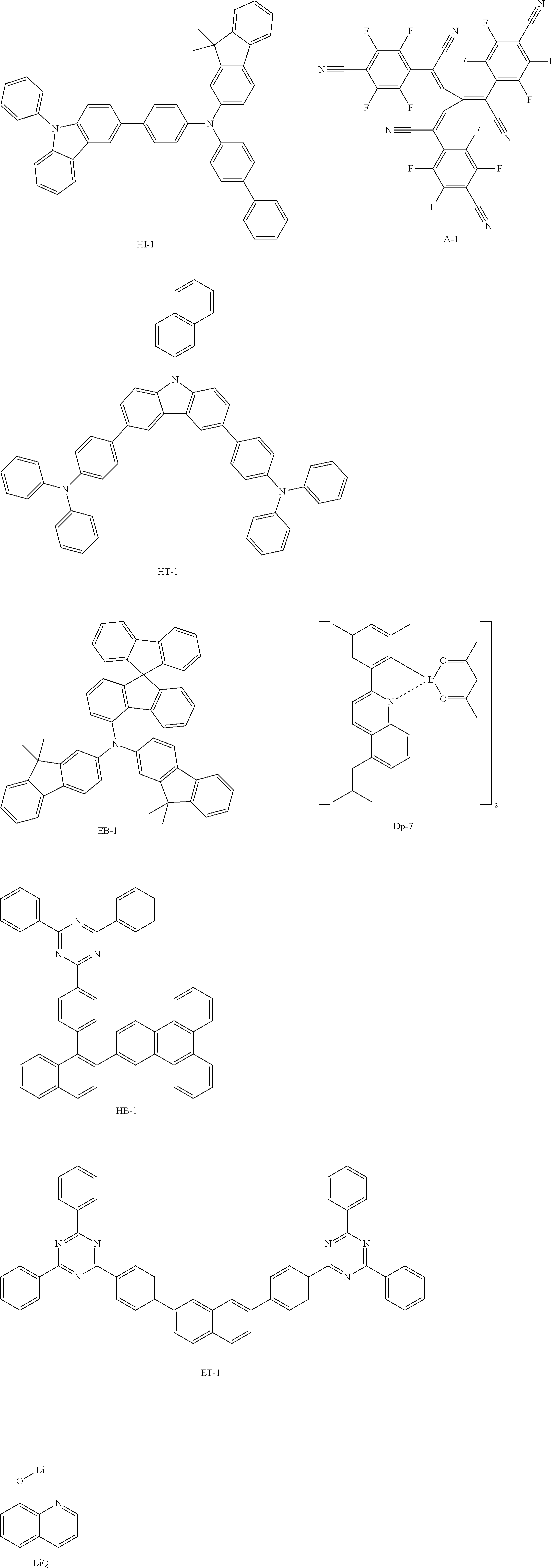

In the above-mentioned processes, the deposition rates of the organic materials were maintained at 0.4-0.7 Å/sec, the deposition rates of lithium fluoride and the aluminum of the cathode were maintained at 0.3 Å/sec and 2 Å/sec, respectively, and the degree of vacuum during the deposition was maintained at 2×10⁻⁷˜ 5×10⁻⁶ torr, thereby manufacturing an organic light emitting device.

### Examples 2 to 580

The organic light emitting devices were manufactured in the same manner as in Example 1, except that in the formation of the light emitting layer, the compounds shown in Table 1 below were used instead of Compound 1-1 and Compound 2-2 as the first and second hosts.

### Comparative Examples 1 to 60

The organic light emitting devices were manufactured in the same manner as in Example 1, except that in the formation of the light emitting layer, the compounds shown in Table 2 below were used instead of Compound 1-1 and Compound 2-2 as the first and second hosts. The structures of Compounds A-1 to A-12 of Table 2 are as follows.

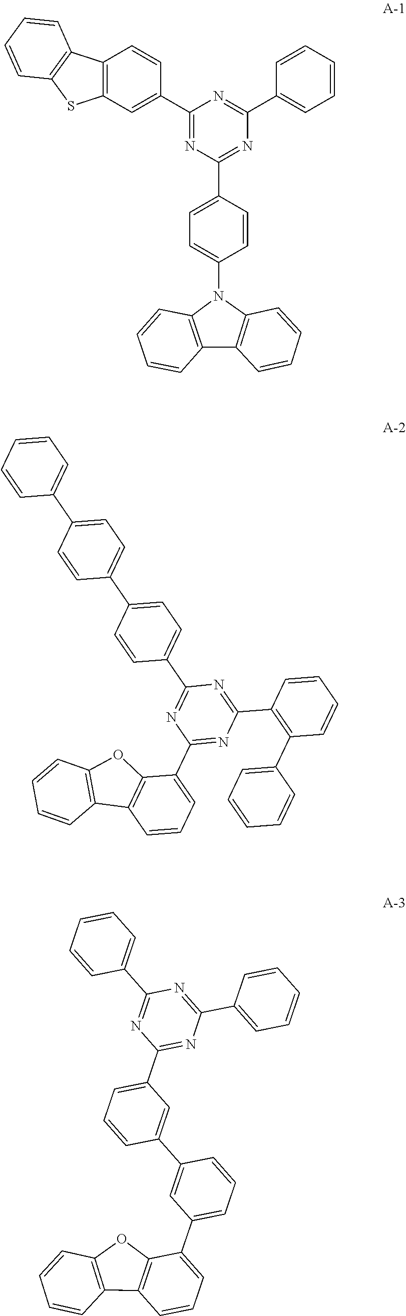 [SMILES: c1ccc(-c2nc(-c3ccc(-n4c5ccccc5c5ccccc54)cc3)nc(-c3ccc4c(c3)sc3ccccc34)n2)cc1 ; c1ccc(-c2ccc(-c3ccc(-c4nc(-c5ccccc5-c5ccccc5)nc(-c5cccc6c5oc5ccccc56)n4)cc3)cc2)cc1 ; c1ccc(-c2nc(-c3ccccc3)nc(-c3cccc(-c4cccc(-c5cccc6c5oc5ccccc56)c4)c3)n2)cc1]

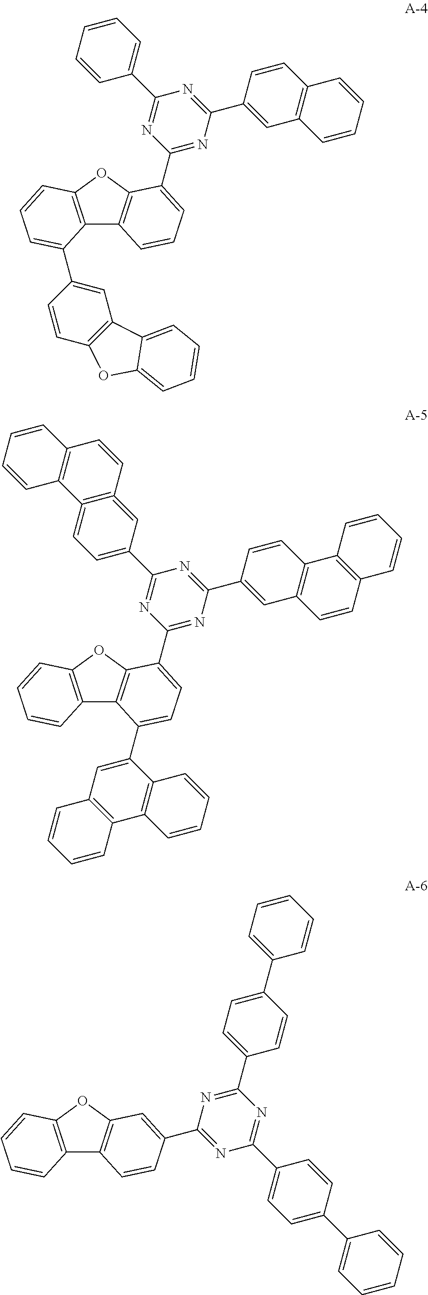

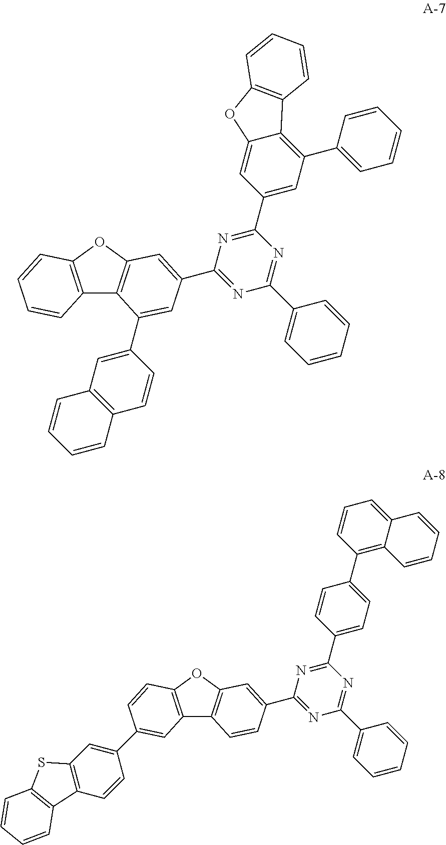 [SMILES: c1ccc(-c2nc(-c3cc(-c4ccccc4)c4c(c3)oc3ccccc34)nc(-c3cc(-c4ccc5ccccc5c4)c4c(c3)oc3ccccc34)n2)cc1 ; c1ccc(-c2nc(-c3ccc(-c4cccc5ccccc45)cc3)nc(-c3ccc4c(c3)oc3ccc(-c5ccc6c(c5)sc5ccccc56)cc34)n2)cc1]

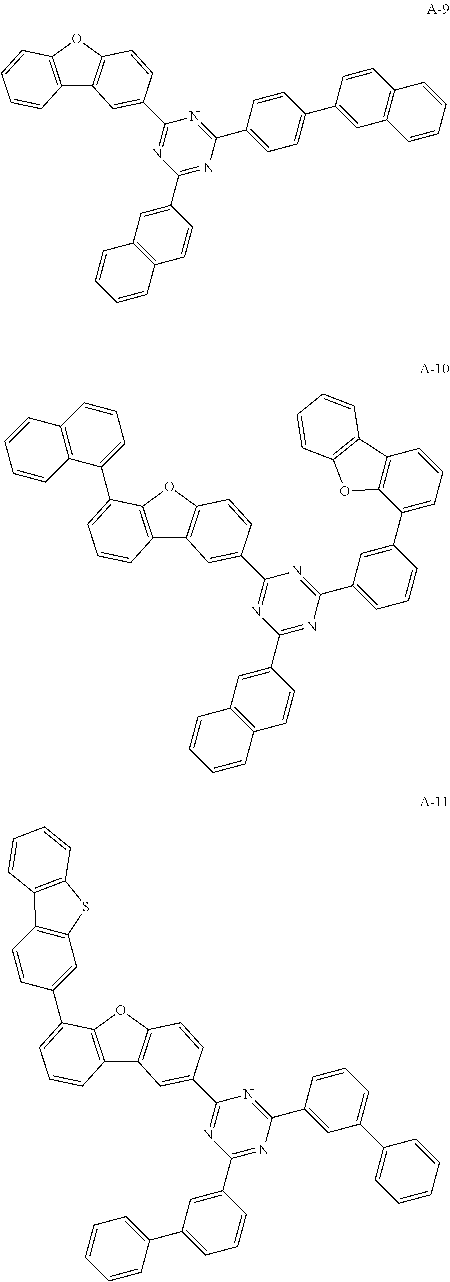 [SMILES: c1ccc2cc(-c3ccc(-c4nc(-c5ccc6ccccc6c5)nc(-c5ccc6oc7ccccc7c6c5)n4)cc3)ccc2c1 ; c1cc(-c2nc(-c3ccc4ccccc4c3)nc(-c3ccc4oc5c(-c6cccc7ccccc67)cccc5c4c3)n2)cc(-c2cccc3c2oc2ccccc23)c1 ; c1ccc(-c2cccc(-c3nc(-c4cccc(-c5ccccc5)c4)nc(-c4ccc5oc6c(-c7ccc8c(c7)sc7ccccc78)cccc6c5c4)n3)c2)cc1]

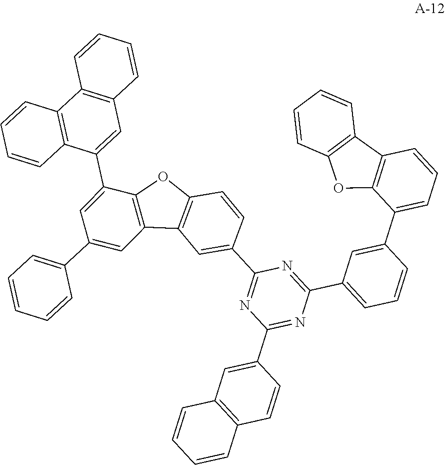 [SMILES: c1ccc(-c2cc(-c3cc4ccccc4c4ccccc34)c3oc4ccc(-c5nc(-c6cccc(-c7cccc8c7oc7ccccc78)c6)nc(-c6ccc7ccccc7c6)n5)cc4c3c2)cc1]

### Comparative Examples 61 to 156

The organic light emitting devices were manufactured in the same manner as in Example 1, except that in the formation of the light emitting layer, the compounds shown in Table 3 below were used instead of Compound 1-1 and Compound 2-2 as the first and second hosts. The structures of Compounds B-1 to B-12 in Table 3 are as follows.

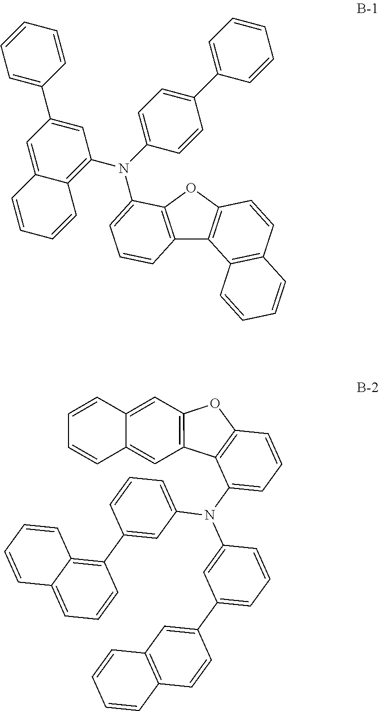 [SMILES: c1ccc(-c2ccc(N(c3cc(-c4ccccc4)cc4ccccc34)c3cccc4c3oc3ccc5ccccc5c34)cc2)cc1 ; c1cc(-c2ccc3ccccc3c2)cc(N(c2cccc(-c3cccc4ccccc34)c2)c2cccc3oc4cc5ccccc5cc4c23)c1]

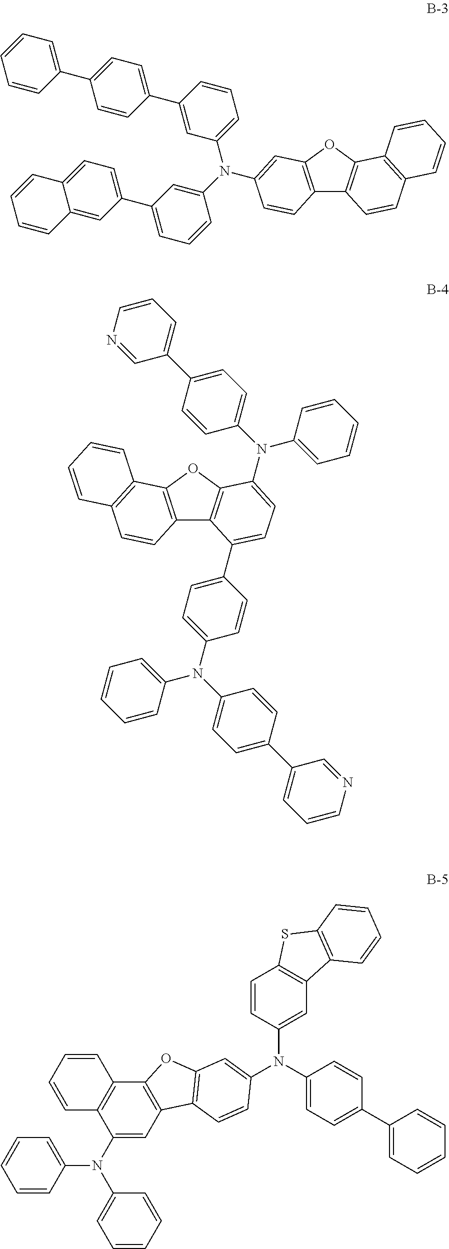 [SMILES: c1ccc(-c2ccc(-c3cccc(N(c4cccc(-c5ccc6ccccc6c5)c4)c4ccc5c(c4)oc4c6ccccc6ccc54)c3)cc2)cc1 ; c1ccc(N(c2ccc(-c3cccnc3)cc2)c2ccc(-c3ccc(N(c4ccccc4)c4ccc(-c5cccnc5)cc4)c4oc5c6ccccc6ccc5c34)cc2)cc1 ; c1ccc(-c2ccc(N(c3ccc4c(c3)oc3c5ccccc5c(N(c5ccccc5)c5ccccc5)cc43)c3ccc4sc5ccccc5c4c3)cc2)cc1]

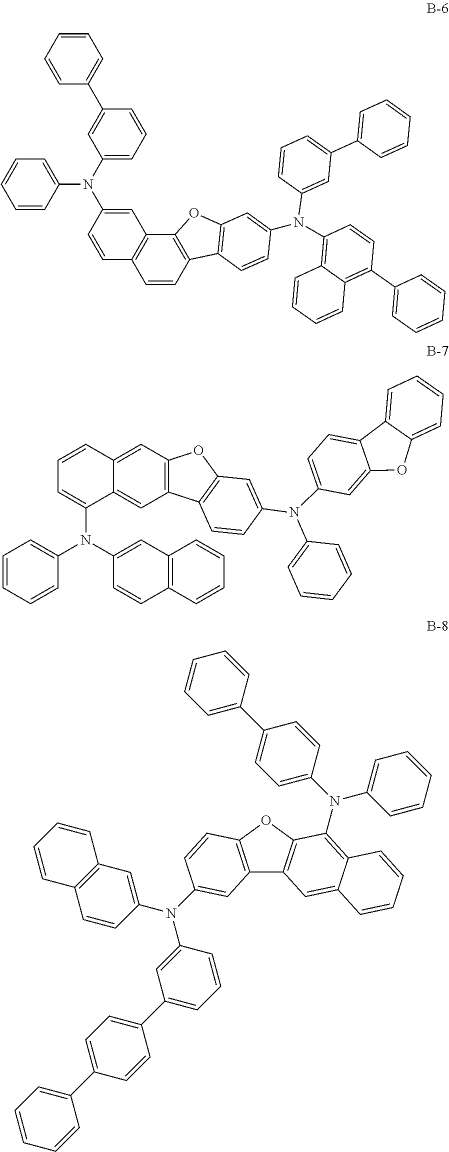

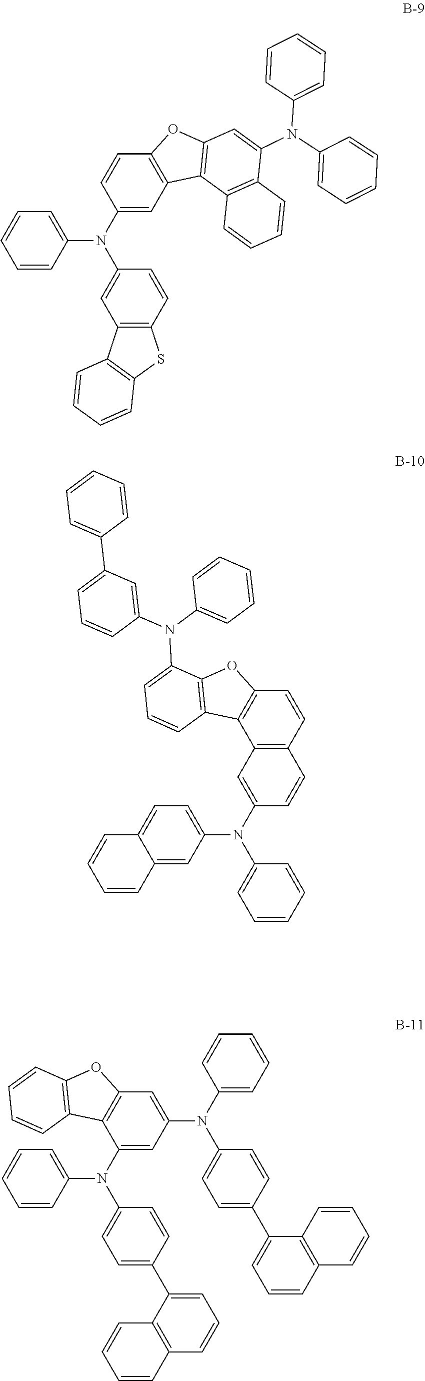

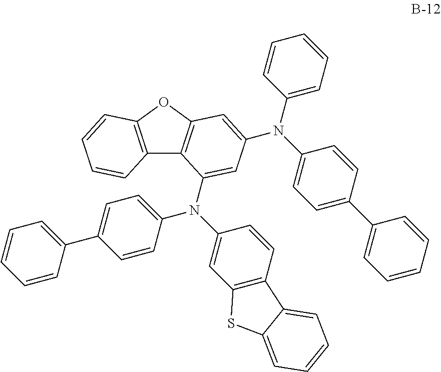 [SMILES: c1ccc(-c2ccc(N(c3ccccc3)c3cc(N(c4ccc(-c5ccccc5)cc4)c4ccc5c(c4)sc4ccccc45)c4c(c3)oc3ccccc34)cc2)cc1]

### Experimental Example

The voltage and efficiency were measured (15 mA/cm²) by applying a current to the organic light emitting devices manufactured in Examples 1 to 580 and Comparative Examples 1 to 156, and the results are shown in Tables 1 to 3 below. Lifetime T95 was measured based on 7000 nits, and means the time (hr) required for the lifetime to be reduced to 95% of the initial lifetime.

TABLE 1

| | | | Driving | Efficiency | Lifespan | Emission |
|---|---|---|---|---|---|---|
| Category | 1st host | 2nd host | voltage(V) | (cd/A) | T95(hr) | color |
| Example 1 | Compound | Compound | 3.58 | 22.74 | 231 | Red |
| | 1-1 | 2-1 | | | | |
| Example 2 | | Compound | 3.55 | 10.94 | 223 | Red |
| | | 2-11 | | | | |
| Example 3 | | Compound | 3.60 | 17.18 | 235 | Red |
| | | 2-21 | | | | |
| Example 4 | | Compound | 3.57 | 12.18 | 239 | Red |
| | | 2-31 | | | | |
| Example 5 | | Compound | 3.58 | 22.62 | 250 | Red |
| | | 2-41 | | | | |
| Example 6 | Compound | Compound | 3.55 | 13.77 | 234 | Red |
| | 1-2 | 2-2 | | | | |
| Example 7 | | Compound | 3.61 | 22.61 | 240 | Red |
| | | 2-12 | | | | |
| Example 8 | | Compound | 3.55 | 22.67 | 244 | Red |
| | | 2-22 | | | | |
| Example 9 | | Compound | 3.52 | 12.01 | 235 | Red |
| | | 2-32 | | | | |
| Example | | Compound | 3.59 | 20.03 | 233 | Red |
| 10 | | 2-42 | | | | |
| Example | Compound | Compound | 3.45 | 22.74 | 231 | Red |
| 11 | 1-3 | 2-3 | | | | |
| Example | | Compound | 3.52 | 22.82 | 223 | Red |
| 12 | | 2-13 | | | | |
| Example | | Compound | 3.47 | 22.83 | 235 | Red |
| 13 | | 2-23 | | | | |
| Example | | Compound | 3.54 | 22.59 | 239 | Red |
| 14 | | 2-33 | | | | |
| Example | | Compound | 3.45 | 22.68 | 250 | Red |
| 15 | | 2-43 | | | | |
| Example | Compound | Compound | 3.52 | 23.67 | 257 | Red |
| 16 | 1-4 | 2-4 | | | | |
| Example | | Compound | 3.49 | 23.30 | 257 | Red |
| 17 | | 2-14 | | | | |
| Example | | Compound | 3.51 | 23.94 | 270 | Red |
| 18 | | 2-24 | | | | |
| Example | | Compound | 3.47 | 22.98 | 260 | Red |
| 19 | | 2-34 | | | | |
| Example | | Compound | 3.51 | 23.29 | 254 | Red |
| 20 | | 2-44 | | | | |
| Example | Compound | Compound | 3.51 | 23.14 | 273 | Red |
| 21 | 1-5 | 2-5 | | | | |
| Example | | Compound | 3.46 | 22.81 | 248 | Red |
| 22 | | 2-15 | | | | |
| Example | | Compound | 3.54 | 23.26 | 266 | Red |
| 23 | | 2-25 | | | | |
| Example | | Compound | 3.50 | 23.30 | 263 | Red |
| 24 | | 2-35 | | | | |
| Example | | Compound | 3.53 | 23.85 | 267 | Red |
| 25 | | 2-45 | | | | |
| Example | Compound | Compound | 3.56 | 22.50 | 251 | Red |
| 26 | 1-6 | 2-6 | | | | |
| Example | | Compound | 3.56 | 22.57 | 229 | Red |
| 27 | | 2-16 | | | | |
| Example | | Compound | 3.54 | 13.45 | 230 | Red |
| 28 | | 2-26 | | | | |
| Example | | Compound | 3.54 | 15.64 | 239 | Red |
| 29 | | 2-36 | | | | |
| Example | | Compound | 3.57 | 12.16 | 248 | Red |
| 30 | | 2-46 | | | | |
| Example | Compound | Compound | 3.59 | 22.56 | 226 | Red |
| 31 | 1-7 | 2-7 | | | | |
| Example | | Compound | 3.60 | 20.25 | 220 | Red |
| 32 | | 2-17 | | | | |
| Example | | Compound | 3.60 | 19.22 | 228 | Red |
| 33 | | 2-27 | | | | |
| Example | | Compound | 3.65 | 15.90 | 217 | Red |
| 34 | | 2-37 | | | | |
| Example | | Compound | 3.65 | 17.14 | 223 | Red |
| 35 | | 2-47 | | | | |
| Example | Compound | Compound | 3.66 | 21.22 | 219 | Red |
| 36 | 1-8 | 2-8 | | | | |
| Example | | Compound | 3.67 | 21.70 | 225 | Red |
| 37 | | 2-18 | | | | |
| Example | | Compound | 3.60 | 20.27 | 214 | Red |
| 38 | | 2-28 | | | | |
| Example | | Compound | 3.62 | 18.54 | 224 | Red |
| 39 | | 2-38 | | | | |
| Example | | Compound | 3.64 | 21.14 | 218 | Red |
| 40 | | 2-48 | | | | |
| Example | Compound | Compound | 3.68 | 20.39 | 206 | Red |
| 41 | 1-9 | 2-9 | | | | |
| Example | | Compound | 3.77 | 21.15 | 192 | Red |
| 42 | | 2-19 | | | | |
| Example | | Compound | 3.70 | 20.33 | 190 | Red |
| 43 | | 2-29 | | | | |
| Example | | Compound | 3.68 | 20.17 | 198 | Red |
| 44 | | 2-39 | | | | |
| Example | | Compound | 3.72 | 20.42 | 193 | Red |
| 45 | | 2-49 | | | | |
| Example | Compound | Compound | 3.52 | 22.59 | 245 | Red |
| 46 | 1-10 | 2-10 | | | | |
| Example | | Compound | 3.53 | 22.65 | 224 | Red |
| 47 | | 2-20 | | | | |
| Example | | Compound | 3.54 | 22.58 | 228 | Red |
| 48 | | 2-30 | | | | |
| Example | | Compound | 3.53 | 22.78 | 225 | Red |
| 49 | | 2-40 | | | | |
| Example | | Compound | 3.47 | 22.61 | 230 | Red |
| 50 | | 2-50 | | | | |
| Example | Compound | Compound | 3.49 | 22.63 | 248 | Red |
| 51 | 1-11 | 2-1 | | | | |
| Example | | Compound | 3.47 | 22.80 | 243 | Red |
| 52 | | 2-11 | | | | |
| Example | | Compound | 3.51 | 22.71 | 242 | Red |
| 53 | | 2-21 | | | | |
| Example | | Compound | 3.50 | 22.50 | 242 | Red |
| 54 | | 2-31 | | | | |
| Example | | Compound | 3.47 | 22.59 | 247 | Red |
| 55 | | 2-41 | | | | |
| Example | Compound | Compound | 3.77 | 21.19 | 198 | Red |
| 56 | 1-12 | 2-2 | | | | |
| Example | | Compound | 3.69 | 21.10 | 207 | Red |
| 57 | | 2-12 | | | | |
| Example | | Compound | 3.70 | 20.93 | 196 | Red |
| 58 | | 2-22 | | | | |
| Example | | Compound | 3.70 | 20.20 | 205 | Red |
| 59 | | 2-32 | | | | |
| Example | | Compound | 3.70 | 20.98 | 198 | Red |
| 60 | | 2-42 | | | | |
| Example | Compound | Compound | 3.68 | 20.73 | 190 | Red |
| 61 | 1-13 | 2-3 | | | | |
| Example | | Compound | 3.77 | 20.92 | 206 | Red |
| 62 | | 2-13 | | | | |
| Example | | Compound | 3.77 | 21.34 | 204 | Red |
| 63 | | 2-23 | | | | |
| Example | | Compound | 3.69 | 21.25 | 200 | Red |
| 64 | | 2-33 | | | | |
| Example | | Compound | 3.65 | 21.15 | 207 | Red |
| 65 | | 2-43 | | | | |
| Example | Compound | Compound | 3.67 | 20.61 | 226 | Red |
| 66 | 1-14 | 2-4 | | | | |
| Example | | Compound | 3.66 | 19.15 | 216 | Red |
| 67 | | 2-14 | | | | |
| Example | | Compound | 3.67 | 22.64 | 223 | Red |
| 68 | | 2-24 | | | | |
| Example | | Compound | 3.62 | 15.17 | 219 | Red |
| 69 | | 2-34 | | | | |
| Example | | Compound | 3.67 | 16.95 | 213 | Red |
| 70 | | 2-44 | | | | |
| Example | Compound | Compound | 3.65 | 18.51 | 211 | Red |
| 71 | 1-15 | 2-5 | | | | |
| Example | | Compound | 3.62 | 17.30 | 223 | Red |
| 72 | | 2-15 | | | | |
| Example | | Compound | 3.62 | 18.52 | 216 | Red |
| 73 | | 2-25 | | | | |
| Example | | Compound | 3.65 | 19.20 | 214 | Red |
| 74 | | 2-35 | | | | |
| Example | | Compound | 3.59 | 22.33 | 220 | Red |
| 75 | | 2-45 | | | | |
| Example | Compound | Compound | 3.54 | 22.61 | 238 | Red |
| 76 | 1-16 | 2-6 | | | | |
| Example | | Compound | 3.47 | 22.57 | 235 | Red |
| 77 | | 2-16 | | | | |
| Example | | Compound | 3.52 | 22.51 | 237 | Red |
| 78 | | 2-26 | | | | |
| Example | | Compound | 3.53 | 22.58 | 225 | Red |
| 79 | | 2-36 | | | | |
| Example | | Compound | 3.47 | 22.59 | 240 | Red |
| 80 | | 2-46 | | | | |
| Example | Compound | Compound | 3.54 | 22.83 | 239 | Red |
| 81 | 1-17 | 2-7 | | | | |
| Example | | Compound | 3.52 | 22.59 | 239 | Red |
| 82 | | 2-17 | | | | |
| Example | | Compound | 3.51 | 22.60 | 228 | Red |
| 83 | | 2-27 | | | | |
| Example | | Compound | 3.53 | 22.89 | 235 | Red |
| 84 | | 2-37 | | | | |
| Example | | Compound | 3.53 | 22.89 | 229 | Red |
| 85 | | 2-47 | | | | |
| Example | Compound | Compound | 3.53 | 22.61 | 238 | Red |
| 86 | 1-18 | 2-8 | | | | |
| Example | | Compound | 3.57 | 17.37 | 235 | Red |
| 87 | | 2-18 | | | | |
| Example | | Compound | 3.56 | 19.39 | 237 | Red |
| 88 | | 2-28 | | | | |
| Example | | Compound | 3.61 | 19.31 | 225 | Red |
| 89 | | 2-38 | | | | |
| Example | | Compound | 3.54 | 22.58 | 240 | Red |
| 90 | | 2-48 | | | | |
| Example | Compound | Compound | 3.60 | 15.09 | 239 | Red |
| 91 | 1-19 | 2-9 | | | | |
| Example | | Compound | 3.57 | 18.60 | 239 | Red |
| 92 | | 2-19 | | | | |
| Example | | Compound | 3.58 | 21.01 | 228 | Red |
| 93 | | 2-29 | | | | |
| Example | | Compound | 3.58 | 13.21 | 235 | Red |
| 94 | | 2-39 | | | | |
| Example | | Compound | 3.57 | 15.54 | 229 | Red |
| 95 | | 2-49 | | | | |
| Example | Compound | Compound | 3.47 | 22.98 | 258 | Red |
| 96 | 1-20 | 2-10 | | | | |
| Example | | Compound | 3.50 | 23.23 | 255 | Red |
| 97 | | 2-20 | | | | |
| Example | | Compound | 3.51 | 23.56 | 272 | Red |
| 98 | | 2-30 | | | | |
| Example | | Compound | 3.51 | 22.83 | 263 | Red |
| 99 | | 2-40 | | | | |
| Example | | Compound | 3.52 | 23.33 | 270 | Red |
| 100 | | 2-50 | | | | |
| Example | Compound | Compound | 3.54 | 22.97 | 257 | Red |
| 101 | 1-21 | 2-1 | | | | |
| Example | | Compound | 3.47 | 23.67 | 264 | Red |
| 102 | | 2-11 | | | | |
| Example | | Compound | 3.48 | 23.77 | 248 | Red |
| 103 | | 2-21 | | | | |
| Example | | Compound | 3.46 | 23.61 | 248 | Red |
| 104 | | 2-31 | | | | |
| Example | | Compound | 3.48 | 22.85 | 260 | Red |
| 105 | | 2-41 | | | | |
| Example | Compound | Compound | 3.52 | 22.54 | 273 | Red |
| 106 | 1-22 | 2-2 | | | | |
| Example | | Compound | 3.48 | 22.79 | 269 | Red |
| 107 | | 2-12 | | | | |
| Example | | Compound | 3.54 | 22.75 | 272 | Red |
| 108 | | 2-22 | | | | |
| Example | | Compound | 3.54 | 22.67 | 247 | Red |
| 109 | | 2-32 | | | | |
| Example | | Compound | 3.52 | 22.77 | 269 | Red |
| 110 | | 2-42 | | | | |
| Example | Compound | Compound | 3.54 | 22.71 | 253 | Red |
| 111 | 1-23 | 2-3 | | | | |
| Example | | Compound | 3.53 | 22.78 | 271 | Red |
| 112 | | 2-13 | | | | |
| Example | | Compound | 3.53 | 22.56 | 272 | Red |
| 113 | | 2-23 | | | | |
| Example | | Compound | 3.48 | 22.86 | 266 | Red |
| 114 | | 2-33 | | | | |
| Example | | Compound | 3.46 | 22.90 | 262 | Red |
| 115 | | 2-43 | | | | |
| Example | Compound | Compound | 3.51 | 22.63 | 250 | Red |
| 116 | 1-24 | 2-4 | | | | |
| Example | | Compound | 3.45 | 22.72 | 223 | Red |
| 117 | | 2-14 | | | | |
| Example | | Compound | 3.47 | 22.88 | 242 | Red |
| 118 | | 2-24 | | | | |
| Example | | Compound | 3.46 | 22.86 | 225 | Red |
| 119 | | 2-34 | | | | |
| Example | | Compound | 3.47 | 22.51 | 230 | Red |
| 120 | | 2-44 | | | | |
| Example | Compound | Compound | 3.69 | 20.45 | 206 | Red |
| 121 | 1-25 | 2-5 | | | | |
| Example | | Compound | 3.66 | 20.67 | 205 | Red |
| 122 | | 2-15 | | | | |
| Example | | Compound | 3.76 | 21.23 | 205 | Red |
| 123 | | 2-25 | | | | |
| Example | | Compound | 3.77 | 21.17 | 197 | Red |
| 124 | | 2-35 | | | | |
| Example | | Compound | 3.68 | 20.10 | 203 | Red |
| 125 | | 2-45 | | | | |
| Example | Compound | Compound | 3.75 | 20.50 | 193 | Red |
| 126 | 1-26 | 2-6 | | | | |
| Example | | Compound | 3.74 | 20.38 | 208 | Red |
| 127 | | 2-16 | | | | |
| Example | | Compound | 3.65 | 21.14 | 206 | Red |
| 128 | | 2-26 | | | | |
| Example | | Compound | 3.70 | 20.65 | 197 | Red |
| 129 | | 2-36 | | | | |
| Example | | Compound | 3.71 | 21.49 | 196 | Red |
| 130 | | 2-46 | | | | |
| Example | Compound | Compound | 3.66 | 12.82 | 226 | Red |
| 131 | 1-27 | 2-7 | | | | |
| Example | | Compound | 3.61 | 11.44 | 217 | Red |
| 132 | | 2-17 | | | | |
| Example | | Compound | 3.59 | 19.04 | 226 | Red |
| 133 | | 2-27 | | | | |
| Example | | Compound | 3.66 | 19.84 | 219 | Red |
| 134 | | 2-37 | | | | |
| Example | | Compound | 3.66 | 15.93 | 213 | Red |
| 135 | | 2-47 | | | | |
| Example | Compound | Compound | 3.59 | 20.43 | 226 | Red |
| 136 | 1-28 | 2-8 | | | | |
| Example | | Compound | 3.64 | 12.56 | 228 | Red |
| 137 | | 2-18 | | | | |
| Example | | Compound | 3.61 | 15.51 | 221 | Red |
| 138 | | 2-28 | | | | |
| Example | | Compound | 3.66 | 12.42 | 222 | Red |
| 139 | | 2-38 | | | | |
| Example | | Compound | 3.66 | 12.91 | 214 | Red |
| 140 | | 2-48 | | | | |
| Example | Compound | Compound | 3.59 | 22.79 | 225 | Red |
| 141 | 1-29 | 2-9 | | | | |
| Example | | Compound | 3.58 | 20.25 | 244 | Red |
| 142 | | 2-19 | | | | |
| Example | | Compound | 3.54 | 14.62 | 229 | Red |
| 143 | | 2-29 | | | | |
| Example | | Compound | 3.61 | 16.42 | 236 | Red |
| 144 | | 2-39 | | | | |
| Example | | Compound | 3.59 | 11.47 | 251 | Red |
| 145 | | 2-49 | | | | |
| Example | Compound | Compound | 3.52 | 16.20 | 245 | Red |
| 146 | 1-30 | 2-10 | | | | |
| Example | | Compound | 3.59 | 17.19 | 249 | Red |
| 147 | | 2-20 | | | | |
| Example | | Compound | 3.60 | 22.77 | 238 | Red |
| 148 | | 2-30 | | | | |
| Example | | Compound | 3.55 | 13.69 | 250 | Red |
| 149 | | 2-40 | | | | |
| Example | | Compound | 3.55 | 20.14 | 245 | Red |
| 150 | | 2-50 | | | | |
| Example | Compound | Compound | 3.72 | 20.73 | 197 | Red |
| 151 | 1-31 | 2-1 | | | | |
| Example | | Compound | 3.72 | 21.20 | 199 | Red |
| 152 | | 2-11 | | | | |
| Example | | Compound | 3.71 | 21.38 | 190 | Red |
| 153 | | 2-21 | | | | |
| Example | | Compound | 3.75 | 20.95 | 206 | Red |
| 154 | | 2-31 | | | | |
| Example | | Compound | 3.76 | 20.50 | 200 | Red |
| 155 | | 2-41 | | | | |
| Example | Compound | Compound | 3.67 | 20.40 | 191 | Red |
| 156 | 1-32 | 2-2 | | | | |
| Example | | Compound | 3.78 | 21.14 | 200 | Red |
| 157 | | 2-12 | | | | |
| Example | | Compound | 3.65 | 20.55 | 198 | Red |
| 158 | | 2-22 | | | | |
| Example | | Compound | 3.77 | 20.11 | 205 | Red |
| 159 | | 2-32 | | | | |
| Example | | Compound | 3.76 | 21.20 | 204 | Red |
| 160 | | 2-42 | | | | |
| Example | Compound | Compound | 3.67 | 18.33 | 223 | Red |
| 161 | 1-33 | 2-3 | | | | |
| Example | | Compound | 3.67 | 20.25 | 212 | Red |
| 162 | | 2-13 | | | | |
| Example | | Compound | 3.60 | 14.62 | 220 | Red |
| 163 | | 2-23 | | | | |
| Example | | Compound | 3.63 | 16.42 | 221 | Red |
| 164 | | 2-33 | | | | |
| Example | | Compound | 3.59 | 11.47 | 214 | Red |
| 165 | | 2-43 | | | | |
| Example | Compound | Compound | 3.52 | 23.67 | 257 | Red |
| 166 | 1-34 | 2-4 | | | | |
| Example | | Compound | 3.49 | 23.30 | 257 | Red |
| 167 | | 2-14 | | | | |
| Example | | Compound | 3.51 | 23.94 | 270 | Red |
| 168 | | 2-24 | | | | |
| Example | | Compound | 3.47 | 22.98 | 260 | Red |
| 169 | | 2-34 | | | | |
| Example | | Compound | 3.51 | 23.29 | 254 | Red |
| 170 | | 2-44 | | | | |
| Example | Compound | Compound | 3.93 | 17.00 | 163 | Red |
| 171 | 1-35 | 2-5 | | | | |
| Example | | Compound | 3.89 | 16.97 | 156 | Red |
| 172 | | 2-15 | | | | |
| Example | | Compound | 3.94 | 17.15 | 162 | Red |
| 173 | | 2-25 | | | | |
| Example | | Compound | 3.89 | 17.41 | 167 | Red |
| 174 | | 2-35 | | | | |
| Example | | Compound | 3.91 | 17.64 | 160 | Red |
| 175 | | 2-45 | | | | |
| Example | Compound | Compound | 3.51 | 22.76 | 230 | Red |
| 176 | 1-36 | 2-6 | | | | |
| Example | | Compound | 3.51 | 22.63 | 224 | Red |
| 177 | | 2-16 | | | | |
| Example | | Compound | 3.47 | 22.62 | 223 | Red |
| 178 | | 2-26 | | | | |
| Example | | Compound | 3.51 | 22.80 | 248 | Red |
| 179 | | 2-36 | | | | |
| Example | | Compound | 3.54 | 22.66 | 224 | Red |
| 180 | | 2-46 | | | | |
| Example | Compound | Compound | 3.56 | 22.54 | 247 | Red |
| 181 | 1-37 | 2-7 | | | | |
| Example | | Compound | 3.57 | 11.68 | 250 | Red |
| 182 | | 2-17 | | | | |
| Example | | Compound | 3.57 | 12.25 | 226 | Red |
| 183 | | 2-27 | | | | |
| Example | | Compound | 3.59 | 15.55 | 236 | Red |
| 184 | | 2-37 | | | | |
| Example | | Compound | 3.54 | 19.92 | 240 | Red |
| 185 | | 2-47 | | | | |
| Example | Compound | Compound | 3.61 | 14.20 | 230 | Red |
| 186 | 1-38 | 2-8 | | | | |
| Example | | Compound | 3.60 | 12.54 | 224 | Red |
| 187 | | 2-18 | | | | |
| Example | | Compound | 3.53 | 14.14 | 223 | Red |
| 188 | | 2-28 | | | | |
| Example | | Compound | 3.57 | 21.49 | 248 | Red |
| 189 | | 2-38 | | | | |
| Example | | Compound | 3.60 | 18.98 | 224 | Red |
| 190 | | 2-48 | | | | |
| Example | Compound | Compound | 3.46 | 23.17 | 248 | Red |
| 191 | 1-39 | 2-9 | | | | |
| Example | | Compound | 3.51 | 23.06 | 250 | Red |
| 192 | | 2-19 | | | | |
| Example | | Compound | 3.46 | 23.32 | 247 | Red |
| 193 | | 2-29 | | | | |
| Example | | Compound | 3.45 | 22.83 | 267 | Red |
| 194 | | 2-39 | | | | |
| Example | | Compound | 3.47 | 23.04 | 262 | Red |
| 195 | | 2-49 | | | | |
| Example | Compound | Compound | 3.50 | 23.06 | 268 | Red |
| 196 | 1-40 | 2-10 | | | | |
| Example | | Compound | 3.53 | 23.90 | 248 | Red |
| 197 | | 2-20 | | | | |
| Example | | Compound | 3.48 | 23.92 | 273 | Red |
| 198 | | 2-30 | | | | |
| Example | | Compound | 3.50 | 23.30 | 256 | Red |
| 199 | | 2-40 | | | | |
| Example | | Compound | 3.49 | 23.65 | 246 | Red |
| 200 | | 2-51 | | | | |
| Example | Compound | Compound | 3.47 | 22.70 | 245 | Red |
| 201 | 1-41 | 2-1 | | | | |
| Example | | Compound | 3.52 | 22.61 | 235 | Red |
| 202 | | 2-11 | | | | |
| Example | | Compound | 3.51 | 22.79 | 231 | Red |
| 203 | | 2-21 | | | | |
| Example | | Compound | 3.53 | 22.63 | 238 | Red |
| 204 | | 2-31 | | | | |
| Example | | Compound | 3.46 | 22.86 | 225 | Red |
| 205 | | 2-41 | | | | |
| Example | Compound | Compound | 3.52 | 22.54 | 251 | Red |
| 206 | 1-42 | 2-2 | | | | |
| Example | | Compound | 3.51 | 22.56 | 229 | Red |
| 207 | | 2-12 | | | | |
| Example | | Compound | 3.51 | 22.62 | 225 | Red |
| 208 | | 2-22 | | | | |
| Example | | Compound | 3.53 | 22.89 | 249 | Red |
| 209 | | 2-32 | | | | |
| Example | | Compound | 3.52 | 22.65 | 236 | Red |
| 210 | | 2-42 | | | | |
| Example | Compound | Compound | 3.60 | 14.63 | 219 | Red |
| 211 | 1-43 | 2-3 | | | | |
| Example | | Compound | 3.62 | 19.89 | 212 | Red |
| 212 | | 2-13 | | | | |
| Example | | Compound | 3.66 | 18.87 | 223 | Red |
| 213 | | 2-23 | | | | |
| Example | | Compound | 3.67 | 12.12 | 215 | Red |
| 214 | | 2-33 | | | | |
| Example | | Compound | 3.63 | 17.28 | 222 | Red |
| 215 | | 2-43 | | | | |
| Example | Compound | Compound | 3.49 | 22.84 | 246 | Red |
| 216 | 1-44 | 2-4 | | | | |
| Example | | Compound | 3.48 | 22.74 | 259 | Red |
| 217 | | 2-14 | | | | |
| Example | | Compound | 3.45 | 22.79 | 255 | Red |
| 218 | | 2-24 | | | | |
| Example | | Compound | 3.47 | 22.50 | 252 | Red |
| 219 | | 2-34 | | | | |
| Example | | Compound | 3.50 | 22.55 | 251 | Red |
| 220 | | 2-44 | | | | |
| Example | Compound | Compound | 3.48 | 22.52 | 258 | Red |
| 221 | 1-45 | 2-5 | | | | |
| Example | | Compound | 3.53 | 22.61 | 252 | Red |
| 222 | | 2-15 | | | | |
| Example | | Compound | 3.48 | 22.88 | 259 | Red |
| 223 | | 2-25 | | | | |
| Example | | Compound | 3.46 | 22.68 | 263 | Red |
| 224 | | 2-35 | | | | |
| Example | | Compound | 3.53 | 22.67 | 264 | Red |
| 225 | | 2-45 | | | | |
| Example | Compound | Compound | 3.49 | 22.84 | 249 | Red |
| 226 | 1-46 | 2-6 | | | | |
| Example | | Compound | 3.48 | 22.74 | 227 | Red |
| 227 | | 2-16 | | | | |
| Example | | Compound | 3.45 | 22.79 | 223 | Red |
| 228 | | 2-26 | | | | |
| Example | | Compound | 3.47 | 22.50 | 224 | Red |
| 229 | | 2-36 | | | | |
| Example | | Compound | 3.50 | 22.55 | 243 | Red |
| 230 | | 2-46 | | | | |
| Example | Compound | Compound | 3.48 | 22.52 | 250 | Red |
| 231 | 1-47 | 2-7 | | | | |
| Example | | Compound | 3.53 | 22.61 | 225 | Red |
| 232 | | 2-17 | | | | |
| Example | | Compound | 3.48 | 22.88 | 225 | Red |
| 233 | | 2-27 | | | | |
| Example | | Compound | 3.46 | 22.68 | 226 | Red |
| 234 | | 2-37 | | | | |
| Example | | Compound | 3.53 | 22.67 | 248 | Red |
| 235 | | 2-47 | | | | |
| Example | Compound | Compound | 3.62 | 19.23 | 225 | Red |
| 236 | 1-48 | 2-8 | | | | |
| Example | | Compound | 3.63 | 13.11 | 212 | Red |
| 237 | | 2-18 | | | | |
| Example | | Compound | 3.66 | 11.02 | 220 | Red |
| 238 | | 2-28 | | | | |
| Example | | Compound | 3.60 | 17.75 | 216 | Red |
| 239 | | 2-38 | | | | |
| Example | | Compound | 3.63 | 12.99 | 226 | Red |
| 240 | | 2-48 | | | | |
| Example | Compound | Compound | 3.61 | 19.47 | 214 | Red |
| 241 | 1-49 | 2-9 | | | | |
| Example | | Compound | 3.66 | 16.80 | 216 | Red |
| 242 | | 2-19 | | | | |
| Example | | Compound | 3.63 | 17.81 | 215 | Red |
| 243 | | 2-29 | | | | |
| Example | | Compound | 3.62 | 13.16 | 222 | Red |
| 244 | | 2-39 | | | | |
| Example | | Compound | 3.64 | 12.97 | 219 | Red |
| 245 | | 2-49 | | | | |
| Example | Compound | Compound | 3.66 | 21.07 | 197 | Red |
| 246 | 1-50 | 2-10 | | | | |
| Example | | Compound | 3.73 | 20.98 | 197 | Red |
| 247 | | 2-20 | | | | |
| Example | | Compound | 3.78 | 21.40 | 208 | Red |
| 248 | | 2-30 | | | | |
| Example | | Compound | 3.71 | 20.46 | 190 | Red |
| 249 | | 2-40 | | | | |
| Example | | Compound | 3.68 | 20.62 | 195 | Red |
| 250 | | 2-50 | | | | |
| Example | Compound | Compound | 3.76 | 21.43 | 198 | Red |
| 251 | 1-51 | 2-1 | | | | |
| Example | | Compound | 3.69 | 21.44 | 195 | Red |
| 252 | | 2-11 | | | | |
| Example | | Compound | 3.71 | 21.07 | 206 | Red |
| 253 | | 2-21 | | | | |
| Example | | Compound | 3.73 | 20.50 | 191 | Red |
| 254 | | 2-31 | | | | |
| Example | | Compound | 3.75 | 20.23 | 200 | Red |
| 255 | | 2-41 | | | | |
| Example | Compound | Compound | 3.64 | 20.39 | 219 | Red |
| 256 | 1-52 | 2-2 | | | | |
| Example | | Compound | 3.60 | 13.37 | 213 | Red |
| 257 | | 2-12 | | | | |
| Example | | Compound | 3.64 | 17.63 | 218 | Red |
| 258 | | 2-22 | | | | |
| Example | | Compound | 3.67 | 19.49 | 219 | Red |
| 259 | | 2-32 | | | | |
| Example | | Compound | 3.67 | 11.18 | 213 | Red |
| 260 | | 2-42 | | | | |
| Example | Compound | Compound | 3.52 | 22.83 | 235 | Red |
| 261 | 1-53 | 2-3 | | | | |
| Example | | Compound | 3.51 | 22.81 | 242 | Red |
| 262 | | 2-13 | | | | |
| Example | | Compound | 3.51 | 22.70 | 238 | Red |
| 263 | | 2-23 | | | | |
| Example | | Compound | 3.48 | 22.57 | 249 | Red |
| 264 | | 2-33 | | | | |
| Example | | Compound | 3.54 | 22.77 | 234 | Red |
| 265 | | 2-43 | | | | |
| Example | Compound | Compound | 3.51 | 22.54 | 235 | Red |
| 266 | 1-54 | 2-4 | | | | |
| Example | | Compound | 3.51 | 22.67 | 240 | Red |
| 267 | | 2-14 | | | | |
| Example | | Compound | 3.52 | 22.70 | 246 | Red |
| 268 | | 2-24 | | | | |
| Example | | Compound | 3.49 | 22.60 | 243 | Red |
| 269 | | 2-34 | | | | |
| Example | | Compound | 3.47 | 22.59 | 232 | Red |
| 270 | | 2-44 | | | | |
| Example | Compound | Compound | 3.75 | 20.72 | 193 | Red |
| 271 | 1-55 | 2-5 | | | | |
| Example | | Compound | 3.78 | 20.84 | 208 | Red |
| 272 | | 2-15 | | | | |
| Example | | Compound | 3.72 | 21.02 | 205 | Red |
| 273 | | 2-25 | | | | |
| Example | | Compound | 3.70 | 21.30 | 208 | Red |
| 274 | | 2-35 | | | | |
| Example | | Compound | 3.72 | 20.76 | 207 | Red |
| 275 | | 2-45 | | | | |
| Example | Compound | Compound | 3.70 | 20.67 | 202 | Red |
| 276 | 1-56 | 2-6 | | | | |
| Example | | Compound | 3.70 | 20.63 | 201 | Red |
| 277 | | 2-16 | | | | |
| Example | | Compound | 3.70 | 21.20 | 194 | Red |
| 278 | | 2-26 | | | | |
| Example | | Compound | 3.77 | 20.13 | 201 | Red |
| 279 | | 2-36 | | | | |
| Example | | Compound | 3.74 | 21.21 | 200 | Red |
| 280 | | 2-46 | | | | |
| Example | Compound | Compound | 3.62 | 11.49 | 220 | Red |
| 281 | 1-57 | 2-7 | | | | |
| Example | | Compound | 3.64 | 15.64 | 228 | Red |
| 282 | | 2-17 | | | | |
| Example | | Compound | 3.63 | 18.28 | 212 | Red |
| 283 | | 2-27 | | | | |
| Example | | Compound | 3.59 | 19.21 | 222 | Red |
| 284 | | 2-37 | | | | |
| Example | | Compound | 3.63 | 13.86 | 224 | Red |
| 285 | | 2-47 | | | | |
| Example | Compound | Compound | 3.65 | 16.15 | 211 | Red |
| 286 | 1-58 | 2-8 | | | | |
| Example | | Compound | 3.63 | 10.96 | 227 | Red |
| 287 | | 2-18 | | | | |
| Example | | Compound | 3.63 | 15.13 | 211 | Red |
| 288 | | 2-28 | | | | |
| Example | | Compound | 3.61 | 12.39 | 227 | Red |
| 289 | | 2-38 | | | | |
| Example | | Compound | 3.62 | 22.74 | 225 | Red |
| 290 | | 2-48 | | | | |
| Example | Compound | Compound | 3.70 | 21.05 | 205 | Red |
| 291 | 1-59 | 2-9 | | | | |
| Example | | Compound | 3.78 | 21.06 | 198 | Red |
| 292 | | 2-19 | | | | |
| Example | | Compound | 3.72 | 21.49 | 192 | Red |
| 293 | | 2-29 | | | | |
| Example | | Compound | 3.73 | 20.84 | 200 | Red |
| 294 | | 2-39 | | | | |
| Example | | Compound | 3.71 | 21.18 | 204 | Red |
| 295 | | 2-49 | | | | |
| Example | Compound | Compound | 3.74 | 21.43 | 194 | Red |
| 296 | 1-60 | 2-10 | | | | |
| Example | | Compound | 3.72 | 21.18 | 192 | Red |
| 297 | | 2-20 | | | | |
| Example | | Compound | 3.65 | 20.94 | 196 | Red |
| 298 | | 2-30 | | | | |
| Example | | Compound | 3.74 | 20.11 | 192 | Red |
| 299 | | 2-40 | | | | |
| Example | | Compound | 3.67 | 20.27 | 201 | Red |
| 300 | | 2-50 | | | | |
| Example | Compound | Compound | 3.51 | 22.50 | 247 | Red |
| 301 | 1-61 | 2-1 | | | | |
| Example | | Compound | 3.47 | 22.80 | 229 | Red |
| 302 | | 2-11 | | | | |
| Example | | Compound | 3.50 | 22.51 | 223 | Red |
| 303 | | 2-21 | | | | |
| Example | | Compound | 3.45 | 22.72 | 236 | Red |
| 304 | | 2-31 | | | | |
| Example | | Compound | 3.50 | 22.60 | 239 | Red |
| 305 | | 2-41 | | | | |
| Example | Compound | Compound | 3.50 | 22.55 | 223 | Red |
| 306 | 1-62 | 2-2 | | | | |
| Example | | Compound | 3.51 | 22.78 | 228 | Red |
| 307 | | 2-12 | | | | |
| Example | | Compound | 3.53 | 22.70 | 232 | Red |
| 308 | | 2-22 | | | | |
| Example | | Compound | 3.52 | 22.83 | 227 | Red |
| 309 | | 2-32 | | | | |
| Example | | Compound | 3.48 | 22.51 | 223 | Red |
| 310 | | 2-42 | | | | |
| Example | Compound | Compound | 3.70 | 21.50 | 195 | Red |
| 311 | 1-63 | 2-3 | | | | |
| Example | | Compound | 3.66 | 20.67 | 191 | Red |
| 312 | | 2-13 | | | | |
| Example | | Compound | 3.77 | 20.80 | 207 | Red |
| 313 | | 2-23 | | | | |
| Example | | Compound | 3.75 | 20.65 | 204 | Red |
| 314 | | 2-33 | | | | |
| Example | | Compound | 3.69 | 20.37 | 208 | Red |
| 315 | | 2-43 | | | | |
| Example | Compound | Compound | 3.74 | 21.37 | 195 | Red |
| 316 | 1-64 | 2-4 | | | | |
| Example | | Compound | 3.76 | 20.33 | 193 | Red |
| 317 | | 2-14 | | | | |
| Example | | Compound | 3.72 | 20.27 | 190 | Red |
| 318 | | 2-24 | | | | |
| Example | | Compound | 3.76 | 20.07 | 203 | Red |
| 319 | | 2-34 | | | | |
| Example | | Compound | 3.75 | 21.23 | 196 | Red |
| 320 | | 2-44 | | | | |
| Example | Compound | Compound | 3.66 | 12.18 | 221 | Red |
| 321 | 1-65 | 2-5 | | | | |
| Example | | Compound | 3.65 | 16.38 | 213 | Red |
| 322 | | 2-15 | | | | |
| Example | | Compound | 3.61 | 12.68 | 226 | Red |
| 323 | | 2-25 | | | | |
| Example | | Compound | 3.59 | 15.68 | 213 | Red |
| 324 | | 2-35 | | | | |
| Example | | Compound | 3.59 | 18.52 | 220 | Red |
| 325 | | 2-45 | | | | |
| Example | Compound | Compound | 3.64 | 16.65 | 215 | Red |
| 326 | 1-66 | 2-6 | | | | |
| Example | | Compound | 3.64 | 20.38 | 227 | Red |
| 327 | | 2-16 | | | | |
| Example | | Compound | 3.63 | 20.99 | 227 | Red |
| 328 | | 2-26 | | | | |
| Example | | Compound | 3.60 | 20.78 | 226 | Red |
| 329 | | 2-36 | | | | |
| Example | | Compound | 3.62 | 21.16 | 215 | Red |
| 330 | | 2-46 | | | | |
| Example | Compound | Compound | 3.54 | 22.65 | 244 | Red |
| 331 | 1-67 | 2-7 | | | | |
| Example | | Compound | 3.47 | 22.68 | 249 | Red |
| 332 | | 2-17 | | | | |
| Example | | Compound | 3.46 | 22.66 | 225 | Red |
| 333 | | 2-27 | | | | |
| Example | | Compound | 3.52 | 22.86 | 246 | Red |
| 334 | | 2-37 | | | | |
| Example | | Compound | 3.53 | 22.87 | 239 | Red |
| 335 | | 2-47 | | | | |
| Example | Compound | Compound | 3.49 | 22.76 | 237 | Red |
| 336 | 1-68 | 2-8 | | | | |
| Example | | Compound | 3.54 | 22.75 | 251 | Red |
| 337 | | 2-18 | | | | |
| Example | | Compound | 3.46 | 22.77 | 226 | Red |
| 338 | | 2-28 | | | | |
| Example | | Compound | 3.50 | 22.81 | 235 | Red |
| 339 | | 2-38 | | | | |
| Example | | Compound | 3.53 | 22.62 | 235 | Red |
| 340 | | 2-48 | | | | |
| Example | Compound | Compound | 3.53 | 22.65 | 244 | Red |
| 341 | 1-69 | 2-9 | | | | |
| Example | | Compound | 3.53 | 11.61 | 249 | Red |
| 342 | | 2-19 | | | | |
| Example | | Compound | 3.55 | 20.75 | 225 | Red |
| 343 | | 2-29 | | | | |
| Example | | Compound | 3.54 | 12.30 | 246 | Red |
| 344 | | 2-39 | | | | |
| Example | | Compound | 3.61 | 20.69 | 239 | Red |
| 345 | | 2-49 | | | | |
| Example | Compound | Compound | 3.58 | 11.26 | 237 | Red |
| 346 | 1-70 | 2-10 | | | | |
| Example | | Compound | 3.61 | 13.11 | 251 | Red |
| 347 | | 2-20 | | | | |
| Example | | Compound | 3.57 | 14.83 | 226 | Red |
| 348 | | 2-30 | | | | |
| Example | | Compound | 3.53 | 13.95 | 235 | Red |
| 349 | | 2-40 | | | | |
| Example | | Compound | 3.52 | 14.73 | 235 | Red |
| 350 | | 2-50 | | | | |
| Example | Compound | Compound | 3.46 | 23.54 | 246 | Red |
| 351 | 1-71 | 2-1 | | | | |
| Example | | Compound | 3.52 | 23.80 | 258 | Red |
| 352 | | 2-11 | | | | |
| Example | | Compound | 3.46 | 23.57 | 268 | Red |
| 353 | | 2-21 | | | | |
| Example | | Compound | 3.49 | 23.11 | 252 | Red |
| 354 | | 2-31 | | | | |
| Example | | Compound | 3.50 | 23.77 | 257 | Red |
| 355 | | 2-41 | | | | |
| Example | Compound | Compound | 3.45 | 23.73 | 260 | Red |
| 356 | 1-72 | 2-2 | | | | |
| Example | | Compound | 3.46 | 23.81 | 252 | Red |
| 357 | | 2-12 | | | | |
| Example | | Compound | 3.47 | 23.89 | 252 | Red |
| 358 | | 2-22 | | | | |
| Example | | Compound | 3.53 | 23.58 | 264 | Red |
| 359 | | 2-32 | | | | |
| Example | | Compound | 3.51 | 23.62 | 255 | Red |
| 360 | | 2-42 | | | | |
| Example | Compound | Compound | 3.61 | 22.62 | 251 | Red |
| 361 | 1-73 | 2-3 | | | | |
| Example | | Compound | 3.53 | 18.45 | 231 | Red |
| 362 | | 2-13 | | | | |
| Example | | Compound | 3.53 | 18.73 | 237 | Red |
| 363 | | 2-23 | | | | |
| Example | | Compound | 3.52 | 20.01 | 239 | Red |
| 364 | | 2-33 | | | | |
| Example | | Compound | 3.59 | 20.51 | 227 | Red |
| 365 | | 2-43 | | | | |
| Example | Compound | Compound | 3.60 | 21.18 | 242 | Red |
| 366 | 1-74 | 2-4 | | | | |
| Example | | Compound | 3.54 | 13.94 | 235 | Red |
| 367 | | 2-14 | | | | |
| Example | | Compound | 3.56 | 21.04 | 235 | Red |
| 368 | | 2-24 | | | | |
| Example | | Compound | 3.52 | 15.48 | 235 | Red |
| 369 | | 2-34 | | | | |
| Example | | Compound | 3.55 | 16.36 | 223 | Red |
| 370 | | 2-44 | | | | |
| Example | Compound | Compound | 3.46 | 22.62 | 246 | Red |
| 371 | 1-75 | 2-5 | | | | |
| Example | | Compound | 3.52 | 22.59 | 258 | Red |
| 372 | | 2-15 | | | | |
| Example | | Compound | 3.46 | 22.86 | 268 | Red |
| 373 | | 2-25 | | | | |
| Example | | Compound | 3.49 | 22.66 | 252 | Red |
| 374 | | 2-35 | | | | |
| Example | | Compound | 3.50 | 22.67 | 257 | Red |
| 375 | | 2-45 | | | | |
| Example | Compound | Compound | 3.45 | 22.53 | 260 | Red |
| 376 | 1-76 | 2-6 | | | | |
| Example | | Compound | 3.46 | 22.76 | 252 | Red |
| 377 | | 2-16 | | | | |
| Example | | Compound | 3.47 | 22.84 | 252 | Red |
| 378 | | 2-26 | | | | |
| Example | | Compound | 3.53 | 22.89 | 264 | Red |
| 379 | | 2-36 | | | | |
| Example | | Compound | 3.51 | 22.82 | 255 | Red |
| 380 | | 2-46 | | | | |
| Example | Compound | Compound | 3.45 | 22.71 | 226 | Red |
| 381 | 1-77 | 2-7 | | | | |
| Example | | Compound | 3.51 | 22.78 | 223 | Red |
| 382 | | 2-17 | | | | |
| Example | | Compound | 3.54 | 22.81 | 225 | Red |
| 383 | | 2-27 | | | | |
| Example | | Compound | 3.48 | 22.80 | 239 | Red |
| 384 | | 2-37 | | | | |
| Example | | Compound | 3.50 | 22.65 | 230 | Red |
| 385 | | 2-47 | | | | |
| Example | Compound | Compound | 3.47 | 22.53 | 247 | Red |
| 386 | 1-78 | 2-8 | | | | |
| Example | | Compound | 3.47 | 22.84 | 237 | Red |
| 387 | | 2-18 | | | | |
| Example | | Compound | 3.49 | 22.90 | 225 | Red |
| 388 | | 2-28 | | | | |
| Example | | Compound | 3.51 | 22.79 | 242 | Red |
| 389 | | 2-38 | | | | |
| Example | | Compound | 3.45 | 22.50 | 224 | Red |
| 380 | | 2-48 | | | | |
| Example | Compound | Compound | 3.54 | 22.71 | 226 | Red |
| 391 | 1-79 | 2-9 | | | | |
| Example | | Compound | 3.59 | 18.77 | 223 | Red |
| 392 | | 2-19 | | | | |
| Example | | Compound | 3.60 | 20.39 | 225 | Red |
| 393 | | 2-29 | | | | |
| Example | | Compound | 3.59 | 22.03 | 239 | Red |
| 394 | | 2-39 | | | | |
| Example | | Compound | 3.57 | 16.42 | 230 | Red |
| 395 | | 2-49 | | | | |
| Example | Compound | Compound | 3.56 | 11.45 | 247 | Red |
| 396 | 1-80 | 2-10 | | | | |
| Example | | Compound | 3.57 | 18.51 | 237 | Red |
| 397 | | 2-20 | | | | |
| Example | | Compound | 3.61 | 20.26 | 225 | Red |
| 398 | | 2-30 | | | | |
| Example | | Compound | 3.61 | 21.32 | 242 | Red |
| 399 | | 2-40 | | | | |
| Example | | Compound | 3.59 | 11.92 | 224 | Red |
| 400 | | 2-52 | | | | |
| Example | Compound | Compound | 3.67 | 20.91 | 205 | Red |
| 401 | 1-81 | 2-1 | | | | |
| Example | | Compound | 3.68 | 20.91 | 203 | Red |
| 402 | | 2-11 | | | | |
| Example | | Compound | 3.68 | 20.11 | 193 | Red |
| 403 | | 2-21 | | | | |
| Example | | Compound | 3.66 | 20.26 | 202 | Red |
| 404 | | 2-31 | | | | |
| Example | | Compound | 3.71 | 20.95 | 193 | Red |
| 405 | | 2-41 | | | | |
| Example | Compound | Compound | 3.70 | 20.49 | 208 | Red |
| 406 | 1-82 | 2-2 | | | | |
| Example | | Compound | 3.77 | 20.45 | 193 | Red |
| 407 | | 2-12 | | | | |
| Example | | Compound | 3.76 | 20.31 | 207 | Red |
| 408 | | 2-22 | | | | |
| Example | | Compound | 3.78 | 20.15 | 207 | Red |
| 409 | | 2-32 | | | | |
| Example | | Compound | 3.77 | 21.14 | 205 | Red |
| 410 | | 2-42 | | | | |
| Example | Compound | Compound | 3.67 | 19.60 | 228 | Red |
| 411 | 1-83 | 2-3 | | | | |
| Example | | Compound | 3.64 | 19.08 | 214 | Red |
| 412 | | 2-13 | | | | |
| Example | | Compound | 3.61 | 13.86 | 212 | Red |
| 413 | | 2-23 | | | | |
| Example | | Compound | 3.63 | 20.81 | 227 | Red |
| 414 | | 2-33 | | | | |
| Example | | Compound | 3.61 | 21.83 | 220 | Red |
| 415 | | 2-43 | | | | |
| Example | Compound | Compound | 3.61 | 14.40 | 218 | Red |
| 416 | 1-84 | 2-4 | | | | |
| Example | | Compound | 3.59 | 13.51 | 224 | Red |
| 417 | | 2-14 | | | | |
| Example | | Compound | 3.61 | 16.46 | 227 | Red |
| 418 | | 2-24 | | | | |
| Example | | Compound | 3.59 | 18.40 | 223 | Red |
| 419 | | 2-34 | | | | |
| Example | | Compound | 3.59 | 12.11 | 213 | Red |
| 420 | | 2-44 | | | | |
| Example | Compound | Compound | 3.53 | 22.69 | 239 | Red |
| 421 | 1-85 | 2-5 | | | | |
| Example | | Compound | 3.47 | 22.52 | 248 | Red |
| 422 | | 2-15 | | | | |
| Example | | Compound | 3.52 | 22.52 | 245 | Red |
| 423 | | 2-25 | | | | |
| Example | | Compound | 3.50 | 22.84 | 232 | Red |
| 424 | | 2-35 | | | | |
| Example | | Compound | 3.46 | 22.74 | 238 | Red |
| 425 | | 2-45 | | | | |
| Example | Compound | Compound | 3.46 | 22.86 | 245 | Red |
| 426 | 1-86 | 2-6 | | | | |
| Example | | Compound | 3.52 | 22.88 | 229 | Red |
| 427 | | 2-16 | | | | |
| Example | | Compound | 3.53 | 22.90 | 250 | Red |
| 428 | | 2-26 | | | | |
| Example | | Compound | 3.54 | 22.83 | 234 | Red |
| 429 | | 2-36 | | | | |
| Example | | Compound | 3.46 | 22.90 | 246 | Red |
| 430 | | 2-46 | | | | |
| Example | Compound | Compound | 3.53 | 23.37 | 271 | Red |
| 431 | 1-87 | 2-7 | | | | |
| Example | | Compound | 3.47 | 23.64 | 254 | Red |
| 432 | | 2-17 | | | | |
| Example | | Compound | 3.52 | 23.80 | 256 | Red |
| 433 | | 2-27 | | | | |
| Example | | Compound | 3.50 | 22.86 | 255 | Red |
| 434 | | 2-37 | | | | |
| Example | | Compound | 3.46 | 23.28 | 257 | Red |
| 435 | | 2-47 | | | | |
| Example | Compound | Compound | 3.46 | 23.61 | 252 | Red |
| 436 | 1-88 | 2-8 | | | | |
| Example | | Compound | 3.52 | 23.12 | 267 | Red |
| 437 | | 2-18 | | | | |
| Example | | Compound | 3.53 | 23.16 | 269 | Red |
| 438 | | 2-28 | | | | |
| Example | | Compound | 3.54 | 23.43 | 260 | Red |
| 439 | | 2-38 | | | | |
| Example | | Compound | 3.46 | 23.43 | 266 | Red |
| 440 | | 2-48 | | | | |
| Example | Compound | Compound | 3.53 | 22.69 | 271 | Red |
| 441 | 1-89 | 2-9 | | | | |
| Example | | Compound | 3.47 | 22.52 | 254 | Red |
| 442 | | 2-19 | | | | |
| Example | | Compound | 3.52 | 22.52 | 256 | Red |
| 443 | | 2-29 | | | | |
| Example | | Compound | 3.50 | 22.84 | 255 | Red |
| 444 | | 2-39 | | | | |
| Example | | Compound | 3.46 | 22.74 | 257 | Red |
| 445 | | 2-49 | | | | |
| Example | Compound | Compound | 3.46 | 22.86 | 252 | Red |
| 446 | 1-90 | 2-10 | | | | |
| Example | | Compound | 3.52 | 22.88 | 267 | Red |
| 447 | | 2-20 | | | | |
| Example | | Compound | 3.53 | 22.90 | 269 | Red |
| 448 | | 2-30 | | | | |
| Example | | Compound | 3.54 | 22.83 | 260 | Red |
| 449 | | 2-40 | | | | |
| Example | | Compound | 3.46 | 22.90 | 266 | Red |
| 450 | | 2-50 | | | | |
| Example | Compound | Compound | 3.63 | 14.38 | 213 | Red |
| 451 | 1-91 | 2-1 | | | | |
| Example | | Compound | 3.60 | 19.80 | 221 | Red |
| 452 | | 2-11 | | | | |
| Example | | Compound | 3.59 | 11.41 | 224 | Red |
| 453 | | 2-21 | | | | |
| Example | | Compound | 3.63 | 21.86 | 211 | Red |
| 454 | | 2-31 | | | | |
| Example | | Compound | 3.61 | 21.33 | 221 | Red |
| 455 | | 2-41 | | | | |
| Example | Compound | Compound | 3.60 | 20.93 | 216 | Red |
| 456 | 1-92 | 2-2 | | | | |
| Example | | Compound | 3.65 | 20.32 | 214 | Red |
| 457 | | 2-12 | | | | |
| Example | | Compound | 3.65 | 12.69 | 223 | Red |
| 458 | | 2-22 | | | | |
| Example | | Compound | 3.65 | 15.54 | 226 | Red |
| 459 | | 2-32 | | | | |
| Example | | Compound | 3.65 | 13.40 | 222 | Red |
| 460 | | 2-42 | | | | |
| Example | Compound | Compound | 3.47 | 22.86 | 245 | Red |
| 461 | 1-93 | 2-3 | | | | |
| Example | | Compound | 3.48 | 22.57 | 250 | Red |
| 462 | | 2-13 | | | | |
| Example | | Compound | 3.47 | 22.85 | 225 | Red |
| 463 | | 2-23 | | | | |
| Example | | Compound | 3.48 | 22.51 | 247 | Red |
| 464 | | 2-33 | | | | |
| Example | | Compound | 3.49 | 22.77 | 235 | Red |
| 465 | | 2-43 | | | | |
| Example | Compound | Compound | 3.49 | 22.76 | 246 | Red |
| 466 | 1-94 | 2-4 | | | | |
| Example | | Compound | 3.48 | 22.80 | 241 | Red |
| 467 | | 2-14 | | | | |
| Example | | Compound | 3.54 | 22.88 | 247 | Red |
| 468 | | 2-24 | | | | |
| Example | | Compound | 3.45 | 22.62 | 227 | Red |
| 469 | | 2-34 | | | | |
| Example | | Compound | 3.51 | 22.84 | 236 | Red |
| 470 | | 2-44 | | | | |
| Example | Compound | Compound | 3.47 | 23.21 | 258 | Red |
| 471 | 1-95 | 2-5 | | | | |
| Example | | Compound | 3.48 | 23.61 | 245 | Red |
| 472 | | 2-15 | | | | |
| Example | | Compound | 3.47 | 22.93 | 269 | Red |
| 473 | | 2-25 | | | | |
| Example | | Compound | 3.48 | 23.95 | 256 | Red |
| 474 | | 2-35 | | | | |
| Example | | Compound | 3.49 | 23.72 | 263 | Red |
| 475 | | 2-45 | | | | |
| Example | Compound | Compound | 3.49 | 23.93 | 246 | Red |
| 476 | 1-96 | 2-6 | | | | |
| Example | | Compound | 3.48 | 23.13 | 245 | Red |
| 477 | | 2-16 | | | | |
| Example | | Compound | 3.54 | 22.81 | 269 | Red |
| 478 | | 2-26 | | | | |
| Example | | Compound | 3.45 | 23.83 | 264 | Red |
| 479 | | 2-36 | | | | |
| Example | | Compound | 3.51 | 23.57 | 253 | Red |
| 480 | | 2-46 | | | | |
| Example | Compound | Compound | 3.47 | 22.86 | 258 | Red |
| 481 | 1-97 | 2-7 | | | | |
| Example | | Compound | 3.48 | 22.57 | 245 | Red |
| 482 | | 2-17 | | | | |
| Example | | Compound | 3.47 | 22.85 | 269 | Red |
| 483 | | 2-27 | | | | |
| Example | | Compound | 3.48 | 22.51 | 256 | Red |
| 484 | | 2-37 | | | | |
| Example | | Compound | 3.49 | 22.77 | 263 | Red |
| 485 | | 2-47 | | | | |
| Example | Compound | Compound | 3.49 | 22.76 | 246 | Red |
| 486 | 1-98 | 2-8 | | | | |
| Example | | Compound | 3.48 | 22.80 | 245 | Red |
| 487 | | 2-18 | | | | |
| Example | | Compound | 3.54 | 22.88 | 269 | Red |
| 488 | | 2-28 | | | | |
| Example | | Compound | 3.45 | 22.62 | 264 | Red |
| 489 | | 2-38 | | | | |
| Example | | Compound | 3.51 | 22.84 | 253 | Red |
| 490 | | 2-48 | | | | |
| Example | Compound | Compound | 3.67 | 20.13 | 223 | Red |
| 491 | 1-99 | 2-9 | | | | |
| Example | | Compound | 3.60 | 17.27 | 228 | Red |
| 492 | | 2-19 | | | | |
| Example | | Compound | 3.62 | 18.21 | 224 | Red |
| 493 | | 2-29 | | | | |
| Example | | Compound | 3.60 | 19.68 | 212 | Red |
| 494 | | 2-39 | | | | |
| Example | | Compound | 3.59 | 15.00 | 226 | Red |
| 495 | | 2-49 | | | | |
| Example | Compound | Compound | 3.65 | 17.75 | 224 | Red |
| 496 | 1-100 | 2-10 | | | | |
| Example | | Compound | 3.64 | 12.54 | 226 | Red |
| 497 | | 2-20 | | | | |
| Example | | Compound | 3.60 | 11.63 | 217 | Red |
| 498 | | 2-30 | | | | |
| Example | | Compound | 3.62 | 14.01 | 219 | Red |
| 499 | | 2-40 | | | | |
| Example | | Compound | 3.65 | 15.82 | 218 | Red |
| 500 | | 2-50 | | | | |
| Example | Compound | Compound | 3.46 | 22.71 | 238 | Red |
| 501 | 1-101 | 2-1 | | | | |
| Example | | Compound | 3.49 | 22.57 | 228 | Red |
| 502 | | 2-11 | | | | |
| Example | | Compound | 3.49 | 22.59 | 249 | Red |
| 503 | | 2-21 | | | | |
| Example | | Compound | 3.47 | 22.70 | 227 | Red |
| 504 | | 2-31 | | | | |
| Example | | Compound | 3.45 | 22.59 | 231 | Red |
| 505 | | 2-41 | | | | |
| Example | Compound | Compound | 3.46 | 22.69 | 225 | Red |
| 506 | 1-102 | 2-2 | | | | |
| Example | | Compound | 3.45 | 22.74 | 232 | Red |
| 507 | | 2-12 | | | | |
| Example | | Compound | 3.45 | 22.88 | 224 | Red |
| 508 | | 2-22 | | | | |
| Example | | Compound | 3.50 | 22.67 | 226 | Red |
| 509 | | 2-32 | | | | |
| Example | | Compound | 3.50 | 22.90 | 225 | Red |
| 510 | | 2-42 | | | | |
| Example | Compound | Compound | 3.75 | 21.48 | 197 | Red |
| 511 | 1-103 | 2-3 | | | | |
| Example | | Compound | 3.73 | 20.54 | 199 | Red |
| 512 | | 2-13 | | | | |
| Example | | Compound | 3.68 | 20.69 | 198 | Red |
| 513 | | 2-23 | | | | |
| Example | | Compound | 3.76 | 21.00 | 190 | Red |
| 514 | | 2-33 | | | | |
| Example | | Compound | 3.68 | 20.76 | 198 | Red |
| 515 | | 2-43 | | | | |
| Example | Compound | Compound | 3.71 | 20.56 | 205 | Red |
| 516 | 1-104 | 2-4 | | | | |
| Example | | Compound | 3.77 | 20.19 | 200 | Red |
| 517 | | 2-14 | | | | |
| Example | | Compound | 3.78 | 20.07 | 197 | Red |
| 518 | | 2-24 | | | | |
| Example | | Compound | 3.70 | 21.12 | 195 | Red |
| 519 | | 2-34 | | | | |
| Example | | Compound | 3.65 | 20.41 | 195 | Red |
| 520 | | 2-44 | | | | |
| Example | Compound | Compound | 3.46 | 23.44 | 262 | Red |
| 521 | | 2-5 | | | | |
| Example | 1-105 | Compound | 3.49 | 22.91 | 272 | Red |
| 522 | | 2-15 | | | | |
| Example | | Compound | 3.49 | 23.85 | 264 | Red |
| 523 | | 2-25 | | | | |
| Example | | Compound | 3.47 | 23.92 | 252 | Red |
| 524 | | 2-35 | | | | |
| Example | | Compound | 3.45 | 23.46 | 258 | Red |
| 525 | | 2-45 | | | | |
| Example | Compound | Compound | 3.46 | 23.91 | 259 | Red |
| 526 | 1-106 | 2-6 | | | | |
| Example | | Compound | 3.45 | 23.57 | 266 | Red |
| 527 | | 2-16 | | | | |
| Example | | Compound | 3.45 | 23.40 | 258 | Red |
| 528 | | 2-26 | | | | |
| Example | | Compound | 3.50 | 23.78 | 273 | Red |
| 529 | | 2-36 | | | | |
| Example | | Compound | 3.50 | 23.47 | 262 | Red |
| 530 | | 2-46 | | | | |
| Example | Compound | Compound | 3.53 | 22.71 | 238 | Red |
| 531 | 1-107 | 2-7 | | | | |
| Example | | Compound | 3.53 | 17.38 | 228 | Red |
| 532 | | 2-17 | | | | |
| Example | | Compound | 3.54 | 20.69 | 249 | Red |
| 533 | | 2-27 | | | | |
| Example | | Compound | 3.52 | 13.48 | 227 | Red |
| 534 | | 2-37 | | | | |
| Example | | Compound | 3.61 | 12.44 | 231 | Red |
| 535 | | 2-47 | | | | |
| Example | Compound | Compound | 3.57 | 20.67 | 225 | Red |
| 536 | 1-108 | 2-8 | | | | |
| Example | | Compound | 3.61 | 15.92 | 232 | Red |
| 537 | | 2-18 | | | | |
| Example | | Compound | 3.57 | 21.18 | 224 | Red |
| 538 | | 2-28 | | | | |
| Example | | Compound | 3.53 | 19.95 | 226 | Red |
| 539 | | 2-38 | | | | |
| Example | | Compound | 3.57 | 15.95 | 225 | Red |
| 540 | | 2-48 | | | | |
| Example | Compound | Compound | 3.53 | 22.79 | 265 | Red |
| 541 | 1-109 | 2-9 | | | | |
| Example | | Compound | 3.46 | 22.76 | 257 | Red |
| 542 | | 2-19 | | | | |
| Example | | Compound | 3.50 | 22.76 | 262 | Red |
| 543 | | 2-29 | | | | |
| Example | | Compound | 3.46 | 22.68 | 268 | Red |
| 544 | | 2-39 | | | | |
| Example | | Compound | 3.50 | 22.77 | 256 | Red |
| 545 | | 2-49 | | | | |
| Example | Compound | Compound | 3.54 | 22.85 | 255 | Red |
| 546 | 1-110 | 2-10 | | | | |
| Example | | Compound | 3.51 | 22.78 | 255 | Red |
| 547 | | 2-20 | | | | |
| Example | | Compound | 3.45 | 22.77 | 270 | Red |
| 548 | | 2-30 | | | | |
| Example | | Compound | 3.50 | 22.58 | 248 | Red |
| 549 | | 2-40 | | | | |
| Example | | Compound | 3.46 | 22.50 | 245 | Red |
| 550 | | 2-50 | | | | |
| Example | Compound | Compound | 3.53 | 22.79 | 226 | Red |
| 551 | 1-111 | 2-1 | | | | |
| Example | | Compound | 3.46 | 22.76 | 245 | Red |
| 552 | | 2-11 | | | | |
| Example | | Compound | 3.50 | 22.76 | 234 | Red |
| 553 | | 2-21 | | | | |
| Example | | Compound | 3.46 | 22.68 | 242 | Red |
| 554 | | 2-31 | | | | |
| Example | | Compound | 3.50 | 22.77 | 240 | Red |
| 555 | | 2-41 | | | | |
| Example | Compound | Compound | 3.54 | 22.85 | 229 | Red |
| 556 | 1-112 | 2-2 | | | | |
| Example | | Compound | 3.51 | 22.78 | 240 | Red |
| 557 | | 2-12 | | | | |
| Example | | Compound | 3.45 | 22.77 | 251 | Red |
| 558 | | 2-22 | | | | |
| Example | | Compound | 3.50 | 22.58 | 240 | Red |
| 559 | | 2-32 | | | | |
| Example | | Compound | 3.46 | 22.50 | 242 | Red |
| 560 | | 2-42 | | | | |
| Example | Compound | Compound | 3.70 | 20.47 | 194 | Red |
| 561 | 1-113 | 2-3 | | | | |
| Example | | Compound | 3.77 | 20.69 | 203 | Red |
| 562 | | 2-13 | | | | |
| Example | | Compound | 3.73 | 21.33 | 197 | Red |
| 563 | | 2-23 | | | | |
| Example | | Compound | 3.74 | 20.94 | 207 | Red |
| 564 | | 2-33 | | | | |
| Example | | Compound | 3.77 | 20.60 | 198 | Red |
| 565 | | 2-43 | | | | |
| Example | Compound | Compound | 3.72 | 20.04 | 205 | Red |
| 566 | 1-114 | 2-4 | | | | |
| Example | | Compound | 3.69 | 20.35 | 206 | Red |
| 567 | | 2-14 | | | | |
| Example | | Compound | 3.69 | 20.34 | 207 | Red |
| 568 | | 2-24 | | | | |
| Example | | Compound | 3.74 | 21.07 | 194 | Red |
| 569 | | 2-34 | | | | |
| Example | | Compound | 3.76 | 21.44 | 190 | Red |
| 570 | | 2-44 | | | | |
| Example | Compound | Compound | 3.60 | 13.99 | 216 | Red |
| 571 | 1-115 | 2-5 | | | | |
| Example | | Compound | 3.62 | 16.54 | 228 | Red |
| 572 | | 2-15 | | | | |
| Example | | Compound | 3.63 | 19.39 | 219 | Red |
| 573 | | 2-25 | | | | |
| Example | | Compound | 3.60 | 21.74 | 227 | Red |
| 574 | | 2-35 | | | | |
| Example | | Compound | 3.62 | 21.52 | 225 | Red |
| 575 | | 2-45 | | | | |
| Example | Compound | Compound | 3.64 | 15.47 | 218 | Red |
| 576 | 1-116 | 2-6 | | | | |
| Example | | Compound | 3.66 | 21.77 | 222 | Red |
| 577 | | 2-16 | | | | |
| Example | | Compound | 3.59 | 22.05 | 224 | Red |
| 578 | | 2-26 | | | | |
| Example | | Compound | 3.66 | 20.10 | 211 | Red |
| 579 | | 2-36 | | | | |
| Example | | Compound | 3.64 | 12.73 | 218 | Red |
| 580 | | 2-46 | | | | |

TABLE 2

| | | | Driving | Efficiency | Lifespan | Emission |
|---|---|---|---|---|---|---|
| Category | 1st host | 2nd host | voltage(V) | (cd/A) | T95(hr) | color |
| Comparative | Compound | Compound | 4.11 | 16.54 | 124 | Red |
| Example 1 | A-1 | 2-1 | | | | |
| Comparative | | Compound | 4.05 | 15.02 | 107 | Red |
| Example 2 | | 2-20 | | | | |
| Comparative | | Compound | 4.15 | 14.57 | 115 | Red |
| Example 3 | | 2-29 | | | | |
| Comparative | | Compound | 4.09 | 15.45 | 122 | Red |
| Example 4 | | 2-37 | | | | |
| Comparative | | Compound | 4.12 | 14.58 | 100 | Red |
| Example 5 | | 2-51 | | | | |
| Comparative | Compound | Compound | 4.15 | 15.54 | 95 | Red |
| Example 6 | A-2 | 2-2 | | | | |
| Comparative | | Compound | 4.09 | 14.84 | 118 | Red |
| Example 7 | | 2-23 | | | | |
| Comparative | | Compound | 4.12 | 15.95 | 113 | Red |
| Example 8 | | 2-30 | | | | |
| Comparative | | Compound | 4.17 | 14.76 | 94 | Red |
| Example 9 | | 2-38 | | | | |
| Comparative | | Compound | 4.08 | 15.08 | 96 | Red |
| Example 10 | | 2-52 | | | | |
| Comparative | Compound | Compound | 4.22 | 16.54 | 92 | Red |
| Example 11 | A-3 | 2-3 | | | | |
| Comparative | | Compound | 4.12 | 15.02 | 89 | Red |
| Example 12 | | 2-16 | | | | |
| Comparative | | Compound | 4.19 | 14.57 | 75 | Red |
| Example 13 | | 2-31 | | | | |
| Comparative | | Compound | 4.21 | 15.45 | 90 | Red |
| Example 14 | | 2-39 | | | | |
| Comparative | | Compound | 4.23 | 14.58 | 82 | Red |
| Example 15 | | 2-46 | | | | |
| Comparative | Compound | Compound | 4.23 | 15.54 | 91 | Red |
| Example 16 | A-4 | 2-4 | | | | |
| Comparative | | Compound | 4.15 | 14.84 | 94 | Red |
| Example 17 | | 2-21 | | | | |
| Comparative | | Compound | 4.17 | 15.95 | 89 | Red |
| Example 18 | | 2-32 | | | | |
| Comparative | | Compound | 4.10 | 14.76 | 88 | Red |
| Example 19 | | 2-40 | | | | |
| Comparative | | Compound | 4.16 | 15.08 | 86 | Red |
| Example 20 | | 2-52 | | | | |
| Comparative | Compound | Compound | 4.11 | 16.94 | 123 | Red |
| Example 21 | A-5 | 2-5 | | | | |
| Comparative | | Compound | 4.05 | 16.84 | 143 | Red |
| Example 22 | | 2-19 | | | | |
| Comparative | | Compound | 4.15 | 16.42 | 127 | Red |
| Example 23 | | 2-33 | | | | |
| Comparative | | Compound | 4.09 | 16.82 | 134 | Red |
| Example 24 | | 2-41 | | | | |
| Comparative | | Compound | 4.12 | 17.13 | 145 | Red |
| Example 25 | | 2-51 | | | | |
| Comparative | Compound | Compound | 4.15 | 17.11 | 141 | Red |
| Example 26 | A-6 | 2-6 | | | | |
| Comparative | | Compound | 4.09 | 16.83 | 129 | Red |
| Example 27 | | 2-17 | | | | |
| Comparative | | Compound | 4.12 | 16.80 | 137 | Red |
| Example 28 | | 2-34 | | | | |
| Comparative | | Compound | 4.17 | 16.68 | 138 | Red |
| Example 29 | | 2-42 | | | | |
| Comparative | | Compound | 4.08 | 16.98 | 146 | Red |
| Example 30 | | 2-49 | | | | |
| Comparative | Compound | Compound | 4.11 | 16.54 | 124 | Red |
| Example 31 | A-7 | 2-7 | | | | |
| Comparative | | Compound | 4.05 | 15.02 | 107 | Red |
| Example 32 | | 2-15 | | | | |
| Comparative | | Compound | 4.15 | 14.57 | 115 | Red |
| Example 33 | | 2-22 | | | | |
| Comparative | | Compound | 4.09 | 15.45 | 122 | Red |
| Example 34 | | 2-39 | | | | |
| Comparative | | Compound | 4.12 | 14.58 | 100 | Red |
| Example 35 | | 2-47 | | | | |
| Comparative | Compound | Compound | 4.15 | 15.54 | 95 | Red |
| Example 36 | A-8 | 2-8 | | | | |
| Comparative | | Compound | 4.09 | 14.84 | 118 | Red |
| Example 37 | | 2-16 | | | | |
| Comparative | | Compound | 4.12 | 15.95 | 113 | Red |
| Example 38 | | 2-25 | | | | |
| Comparative | | Compound | 4.17 | 14.76 | 94 | Red |
| Example 39 | | 2-34 | | | | |
| Comparative | | Compound | 4.08 | 15.08 | 96 | Red |
| Example 40 | | 2-48 | | | | |
| Comparative | Compound | Compound | 3.92 | 16.94 | 123 | Red |
| Example 41 | A-9 | 2-9 | | | | |
| Comparative | | Compound | 3.93 | 16.84 | 143 | Red |
| Example 42 | | 2-18 | | | | |
| Comparative | | Compound | 3.91 | 16.42 | 127 | Red |
| Example 43 | | 2-22 | | | | |
| Comparative | | Compound | 3.91 | 16.82 | 134 | Red |
| Example 44 | | 2-35 | | | | |
| Comparative | | Compound | 3.90 | 17.13 | 145 | Red |
| Example 45 | | 2-50 | | | | |
| Comparative | Compound | Compound | 3.90 | 17.11 | 141 | Red |
| Example 46 | A-10 | 2-10 | | | | |
| Comparative | | Compound | 3.91 | 16.83 | 129 | Red |
| Example 47 | | 2-14 | | | | |
| Comparative | | Compound | 3.89 | 16.80 | 137 | Red |
| Example 48 | | 2-22 | | | | |
| Comparative | | Compound | 3.89 | 16.68 | 138 | Red |
| Example 49 | | 2-39 | | | | |
| Comparative | | Compound | 3.88 | 16.98 | 146 | Red |
| Example 50 | | 2-45 | | | | |
| Comparative | Compound | Compound | 4.07 | 15.08 | 97 | Red |
| Example 51 | A-11 | 2-3 | | | | |
| Comparative | | Compound | 4.10 | 16.01 | 113 | Red |
| Example 52 | | 2-15 | | | | |
| Comparative | | Compound | 4.10 | 16.14 | 105 | Red |
| Example 53 | | 2-27 | | | | |
| Comparative | | Compound | 4.17 | 14.56 | 121 | Red |
| Example 54 | | 2-36 | | | | |
| Comparative | | Compound | 4.16 | 16.19 | 93 | Red |
| Example 55 | | 2-50 | | | | |
| Comparative | Compound | Compound | 4.14 | 16.19 | 125 | Red |
| Example 56 | A-12 | 2-12 | | | | |
| Comparative | | Compound | 4.10 | 16.60 | 101 | Red |
| Example 57 | | 2-26 | | | | |
| Comparative | | Compound | 4.05 | 16.49 | 113 | Red |
| Example 58 | | 2-31 | | | | |
| Comparative | | Compound | 4.09 | 15.69 | 108 | Red |
| Example 59 | | 2-47 | | | | |
| Comparative | | Compound | 4.07 | 15.95 | 105 | Red |
| Example 60 | | 2-51 | | | | |

TABLE 3

| | | | Driving | Efficiency | Lifespan | Emission |
|---|---|---|---|---|---|---|
| Category | 1st host | 2nd host | voltage(V) | (cd/A) | T95(hr) | color |
| Comparative | Compound | Compound | 4.11 | 15.05 | 74 | Red |
| Example 61 | 1-1 | B-1 | | | | |
| Comparative | Compound | | 4.09 | 14.96 | 78 | Red |
| Example 62 | 1-7 | | | | | |
| Comparative | Compound | | 4.09 | 15.02 | 85 | Red |
| Example 63 | 1-16 | | | | | |
| Comparative | Compound | | 4.18 | 15.12 | 93 | Red |
| Example 64 | 1-28 | | | | | |
| Comparative | Compound | | 4.13 | 15.73 | 78 | Red |
| Example 65 | 1-35 | | | | | |
| Comparative | Compound | | 4.23 | 15.70 | 74 | Red |
| Example 66 | 1-43 | | | | | |
| Comparative | Compound | | 4.12 | 15.81 | 96 | Red |
| Example 67 | 1-57 | | | | | |
| Comparative | Compound | | 4.20 | 15.54 | 73 | Red |
| Example 68 | 1-72 | | | | | |
| Comparative | Compound | Compound | 4.11 | 15.05 | 74 | Red |
| Example 69 | 1-2 | B-2 | | | | |
| Comparative | Compound | | 4.09 | 14.96 | 78 | Red |
| Example 70 | 1-10 | | | | | |
| Comparative | Compound | | 4.09 | 15.02 | 85 | Red |
| Example 71 | 1-19 | | | | | |
| Comparative | Compound | | 4.18 | 15.12 | 93 | Red |
| Example 72 | 1-36 | | | | | |
| Comparative | Compound | | 4.13 | 15.73 | 78 | Red |
| Example 73 | 1-51 | | | | | |
| Comparative | Compound | | 4.23 | 15.70 | 74 | Red |
| Example 74 | 1-74 | | | | | |
| Comparative | Compound | | 4.12 | 15.81 | 96 | Red |
| Example 75 | 1-96 | | | | | |
| Comparative | Compound | | 4.20 | 15.54 | 73 | Red |
| Example 76 | 1-108 | | | | | |
| Comparative | Compound | Compound | 4.17 | 15.05 | 99 | Red |
| Example 77 | 1-3 | B-3 | | | | |
| Comparative | Compound | | 4.11 | 14.96 | 121 | Red |
| Example 78 | 1-12 | | | | | |
| Comparative | Compound | | 4.17 | 15.02 | 105 | Red |
| Example 79 | 1-24 | | | | | |
| Comparative | Compound | | 4.14 | 15.12 | 121 | Red |
| Example 80 | 1-57 | | | | | |
| Comparative | Compound | | 4.12 | 15.73 | 124 | Red |
| Example 81 | 1-72 | | | | | |
| Comparative | Compound | | 4.13 | 15.70 | 126 | Red |
| Example 82 | 1-80 | | | | | |
| Comparative | Compound | | 4.09 | 15.81 | 109 | Red |
| Example 83 | 1-94 | | | | | |
| Comparative | Compound | | 4.16 | 15.54 | 123 | Red |
| Example 84 | 1-106 | | | | | |
| Comparative | Compound | Compound | 4.11 | 15.05 | 74 | Red |
| Example 85 | 1-4 | B-4 | | | | |
| Comparative | Compound | | 4.09 | 14.96 | 78 | Red |
| Example 86 | 1-11 | | | | | |
| Comparative | Compound | | 4.09 | 15.02 | 85 | Red |
| Example 87 | 1-23 | | | | | |
| Comparative | Compound | | 4.18 | 15.12 | 93 | Red |
| Example 88 | 1-46 | | | | | |
| Comparative | Compound | | 4.13 | 15.73 | 78 | Red |
| Example 89 | 1-54 | | | | | |
| Comparative | Compound | | 4.23 | 15.70 | 74 | Red |
| Example 90 | 1-83 | | | | | |
| Comparative | Compound | | 4.12 | 15.81 | 96 | Red |
| Example 91 | 1-99 | | | | | |
| Comparative | Compound | | 4.20 | 15.54 | 73 | Red |
| Example 92 | 1-105 | | | | | |
| Comparative | Compound | Compound | 4.17 | 15.05 | 99 | Red |
| Example 93 | 1-5 | B-5 | | | | |
| Comparative | Compound | | 4.11 | 14.96 | 121 | Red |
| Example 94 | 1-14 | | | | | |
| Comparative | Compound | | 4.17 | 15.02 | 105 | Red |
| Example 95 | 1-30 | | | | | |
| Comparative | Compound | | 4.14 | 15.12 | 121 | Red |
| Example 96 | 1-43 | | | | | |
| Comparative | Compound | | 4.12 | 15.73 | 124 | Red |
| Example 97 | 1-55 | | | | | |
| Comparative | Compound | | 4.13 | 15.70 | 126 | Red |
| Example 98 | 1-62 | | | | | |
| Comparative | Compound | | 4.09 | 15.81 | 109 | Red |
| Example 99 | 1-70 | | | | | |
| Comparative | Compound | | 4.16 | 15.54 | 123 | Red |
| Example 100 | 1-97 | | | | | |
| Comparative | Compound | Compound | 4.17 | 17.20 | 138 | Red |
| Example 101 | 1-6 | B-6 | | | | |
| Comparative | Compound | | 4.11 | 17.18 | 133 | Red |
| Example 102 | 1-13 | | | | | |
| Comparative | Compound | | 4.17 | 16.45 | 143 | Red |
| Example 103 | 1-21 | | | | | |
| Comparative | Compound | | 4.14 | 16.65 | 142 | Red |
| Example 104 | 1-42 | | | | | |
| Comparative | Compound | | 4.12 | 16.63 | 121 | Red |
| Example 105 | 1-50 | | | | | |
| Comparative | Compound | | 4.13 | 17.04 | 140 | Red |
| Example 106 | 1-71 | | | | | |
| Comparative | Compound | | 4.09 | 16.42 | 127 | Red |
| Example 107 | 1-87 | | | | | |
| Comparative | Compound | | 4.16 | 16.60 | 145 | Red |
| Example 108 | 1-99 | | | | | |
| Comparative | Compound | Compound | 3.96 | 17.16 | 149 | Red |
| Example 109 | 1-7 | B-7 | | | | |
| Comparative | Compound | | 3.94 | 17.02 | 162 | Red |
| Example 110 | 1-26 | | | | | |
| Comparative | Compound | | 3.93 | 17.38 | 145 | Red |
| Example 111 | 1-35 | | | | | |
| Comparative | Compound | | 3.92 | 17.80 | 146 | Red |
| Example 112 | 1-44 | | | | | |
| Comparative | Compound | | 3.90 | 17.03 | 162 | Red |
| Example 113 | 1-56 | | | | | |
| Comparative | Compound | | 3.93 | 17.20 | 151 | Red |
| Example 114 | 1-78 | | | | | |
| Comparative | Compound | | 3.94 | 17.20 | 157 | Red |
| Example 115 | 1-83 | | | | | |
| Comparative | Compound | | 3.90 | 17.17 | 149 | Red |
| Example 116 | 1-92 | | | | | |
| Comparative | Compound | Compound | 4.15 | 15.38 | 104 | Red |
| Example 117 | 1-18 | B-8 | | | | |
| Comparative | Compound | | 4.11 | 14.68 | 99 | Red |
| Example 118 | 1-27 | | | | | |
| Comparative | Compound | | 4.07 | 15.25 | 112 | Red |
| Example 119 | 1-39 | | | | | |
| Comparative | Compound | | 4.08 | 15.82 | 109 | Red |
| Example 120 | 1-48 | | | | | |
| Comparative | Compound | | 4.13 | 15.75 | 101 | Red |
| Example 121 | 1-78 | | | | | |
| Comparative | Compound | | 4.13 | 14.64 | 102 | Red |
| Example 122 | 1-85 | | | | | |
| Comparative | Compound | | 4.05 | 15.11 | 94 | Red |
| Example 123 | 1-92 | | | | | |
| Comparative | Compound | | 4.07 | 15.44 | 109 | Red |
| Example 124 | 1-103 | | | | | |
| Comparative | Compound | Compound | 4.12 | 17.42 | 160 | Red |
| Example 125 | 1-9 | B-9 | | | | |
| Comparative | Compound | | 3.93 | 16.94 | 148 | Red |
| Example 126 | 1-28 | | | | | |
| Comparative | Compound | | 3.92 | 17.51 | 168 | Red |
| Example 127 | 1-32 | | | | | |
| Comparative | Compound | | 3.91 | 17.30 | 146 | Red |
| Example 128 | 1-50 | | | | | |
| Comparative | Compound | | 3.89 | 16.97 | 147 | Red |
| Example 129 | 1-61 | | | | | |
| Comparative | Compound | | 3.93 | 17.06 | 170 | Red |
| Example 130 | 1-76 | | | | | |
| Comparative | Compound | | 3.94 | 17.76 | 148 | Red |
| Example 131 | 1-85 | | | | | |
| Comparative | Compound | | 3.90 | 17.75 | 170 | Red |
| Example 132 | 1-104 | | | | | |
| Comparative | Compound | Compound | 4.12 | 16.76 | 138 | Red |
| Example 133 | 1-11 | B-10 | | | | |
| Comparative | Compound | | 4.09 | 16.47 | 131 | Red |
| Example 134 | 1-35 | | | | | |
| Comparative | Compound | | 4.15 | 17.20 | 121 | Red |
| Example 135 | 1-46 | | | | | |
| Comparative | Compound | | 4.15 | 16.63 | 137 | Red |
| Example 136 | 1-55 | | | | | |
| Comparative | Compound | | 4.15 | 16.96 | 136 | Red |
| Example 137 | 1-69 | | | | | |
| Comparative | Compound | | 4.13 | 16.55 | 134 | Red |
| Example 138 | 1-89 | | | | | |
| Comparative | Compound | | 4.14 | 16.52 | 125 | Red |
| Example 139 | 1-98 | | | | | |
| Comparative | Compound | | 4.07 | 16.53 | 146 | Red |
| Example 140 | 1-101 | | | | | |
| Comparative | Compound | Compound | 4.16 | 14.99 | 91 | Red |
| Example 141 | 1-3 | B-11 | | | | |
| Comparative | Compound | | 4.09 | 15.31 | 86 | Red |
| Example 142 | 1-14 | | | | | |
| Comparative | Compound | | 4.09 | 15.19 | 73 | Red |
| Example 143 | 1-37 | | | | | |
| Comparative | Compound | | 4.11 | 15.48 | 82 | Red |
| Example 144 | 1-59 | | | | | |
| Comparative | Compound | | 4.17 | 14.69 | 73 | Red |
| Example 145 | 1-67 | | | | | |
| Comparative | Compound | | 4.20 | 14.76 | 90 | Red |
| Example 146 | 1-83 | | | | | |
| Comparative | Compound | | 4.20 | 15.68 | 80 | Red |
| Example 147 | 1-91 | | | | | |
| Comparative | Compound | | 4.16 | 14.98 | 96 | Red |
| Example 148 | 1-100 | | | | | |
| Comparative | Compound | Compound | 4.13 | 16.17 | 92 | Red |
| Example 149 | 1-7 | B-12 | | | | |
| Comparative | Compound | | 4.12 | 16.33 | 102 | Red |
| Example 150 | 1-19 | | | | | |
| Comparative | Compound | | 4.10 | 15.53 | 121 | Red |
| Example 151 | 1-35 | | | | | |
| Comparative | Compound | | 4.15 | 16.25 | 98 | Red |
| Example 152 | 1-52 | | | | | |
| Comparative | Compound | | 4.14 | 15.55 | 96 | Red |
| Example 153 | 1-66 | | | | | |
| Comparative | Compound | | 4.11 | 14.60 | 106 | Red |
| Example 154 | 1-78 | | | | | |
| Comparative | Compound | | 4.16 | 15.13 | 98 | Red |
| Example 155 | 1-85 | | | | | |
| Comparative | Compound | | 4.14 | 15.50 | 100 | Red |
| Example 156 | 1-92 | | | | | |

When a current was applied to the organic light emitting devices manufactured in Examples 1 to 580 and Comparative Examples 1 to 156, the results shown in Tables 1 to 3 were obtained.

Referring to Table 1, it can be confirmed that the organic light emitting devices of the present disclosure, in which the compounds of Chemical Formulas 1 and 2 are co-deposited as a host for the light emitting layer, have a low driving voltage and high efficiency and lifetime. Meanwhile, referring to Table 2, it can be confirmed that when Compounds A-1 to A-12 are used instead of the compound of Chemical Formula 1 as the first host, the driving voltage increases and the efficiency and lifetimes decrease as compared with the devices of Examples, as can be seen in Table 2. Further, referring to Table 3, it can be confirmed that even when Compounds B-1 to B-12 are used instead of the compound of Chemical Formula 2, the driving voltage, efficiency, and lifetime characteristics of the device are also inferior.

From the above results, it can be confirmed that when the compounds of Chemical Formulas 1 and 2 are co-deposited as the first and second hosts, it is advantageous for energy transfer to the dopant in the red light emitting layer as compared with the combination of the compounds of Comparative Examples, thereby capable of improving the driving voltage, luminous efficiency, and lifetime characteristics of the organic light emitting device.

### DESCRIPTION OF REFERENCE NUMERALS

1: substrate
2: anode
3: light emitting layer
4: cathode
5: hole injection layer
6: hole transport layer
7: electron transport layer
8: electron injection layer
9: electron blocking layer
10: hole blocking layer
11: electron injection and transport layer

1. An organic light emitting device comprising:
an anode;
a cathode; and
a light emitting layer between the anode and the cathode,
wherein the light emitting layer includes a compound represented by the following Chemical Formula 1 and a compound represented by the following Chemical Formula 2, 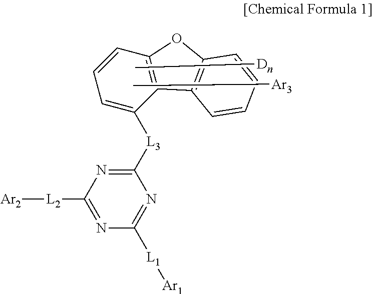
in Chemical Formula 1,
L₁ to L₃ are a single bond; or a substituted or unsubstituted C₆₋₆₀ arylene,
Ar₁ and Ar₂ are each independently a substituted or unsubstituted C₆₋₆₀ aryl; or a substituted or unsubstituted C₂₋₆₀ heteroaryl containing one or more selected from the group consisting of N, O and S,
Ar₃ is hydrogen; deuterium; a substituted or unsubstituted C₆₋₆₀ aryl; or a substituted or unsubstituted C₂₋₆₀ heteroaryl containing one or more selected from the group consisting of N, O and S,
D is deuterium, and
n is an integer of 0 to 6, 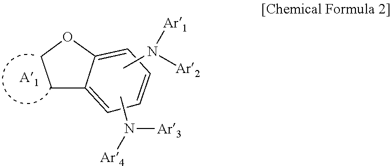
in Chemical Formula 2,
A′₁ is a naphthalene ring, and
Ar′₁ to Ar′₄ are each independently a substituted or unsubstituted C₆₋₆₀ aryl; or a substituted or unsubstituted C₂₋₆₀ heteroaryl containing one or more heteroatoms selected from the group consisting of N, O and S.

2. The organic light emitting device according to claim 1, wherein
L₃ is a single bond; phenylene unsubstituted or substituted with one or more deuteriums; or naphthalenediyl unsubstituted or substituted with one or more deuteriums.

3. The organic light emitting device according to claim 1, wherein
L₁ and L₂ are each independently a single bond; phenylene unsubstituted or substituted with one or more deuteriums; biphenyldiyl unsubstituted or substituted with one or more deuteriums; or naphthalenediyl unsubstituted or substituted with one or more deuteriums.

4. The organic light emitting device according to claim 1, wherein
Ar₁ and Ar₂ are each independently phenyl unsubstituted or substituted with one or more deuteriums; phenyl substituted with triphenylsilyl; biphenylyl unsubstituted or substituted with one or more deuteriums; terphenylyl unsubstituted or substituted with one or more deuteriums; naphthyl unsubstituted or substituted with one or more deuteriums; phenanthrenyl unsubstituted or substituted with one or more deuteriums; dibenzofuranyl unsubstituted or substituted with one or more deuteriums; or dibenzothiophenyl unsubstituted or substituted with one or more deuteriums.

5. The organic light emitting device according to claim 1, wherein:
Ar₃ is hydrogen; deuterium; phenyl unsubstituted or substituted with one or more deuteriums; biphenylyl unsubstituted or substituted with one or more deuteriums; terphenylyl unsubstituted or substituted with one or more deuteriums; naphthyl unsubstituted or substituted with one or more deuteriums; phenanthrenyl unsubstituted or substituted with one or more deuteriums; fluoranthenyl unsubstituted or substituted with one or more deuteriums; phenylnaphthyl unsubstituted or substituted with one or more deuteriums; naphthylphenyl unsubstituted or substituted with one or more deuteriums; triphenylenyl unsubstituted or substituted with one or more deuteriums; dibenzofuranyl unsubstituted or substituted with one or more deuteriums; dibenzothiophenyl unsubstituted or substituted with one or more deuteriums; benzonaphthofuranyl unsubstituted or substituted with one or more deuteriums; or benzonaphthothiophenyl unsubstituted or substituted with one or more deuteriums.

6. The organic light emitting device according to claim 1, wherein
the compound represented by Chemical Formulas 1 is one selected from the following:

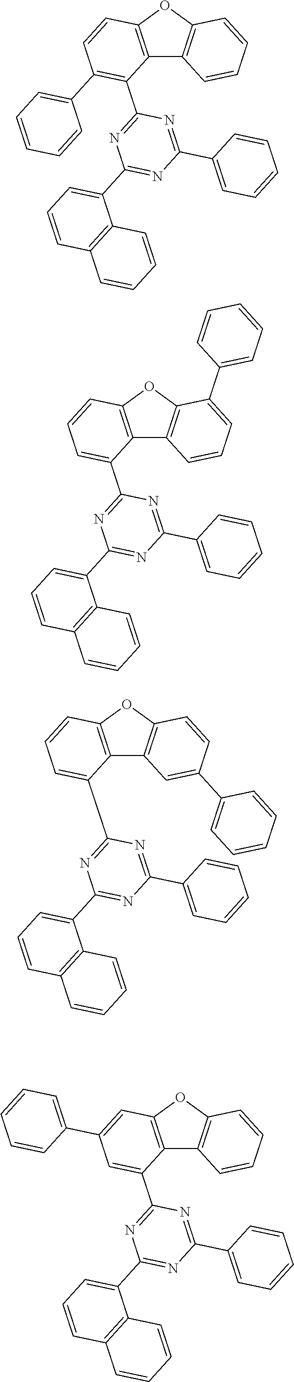

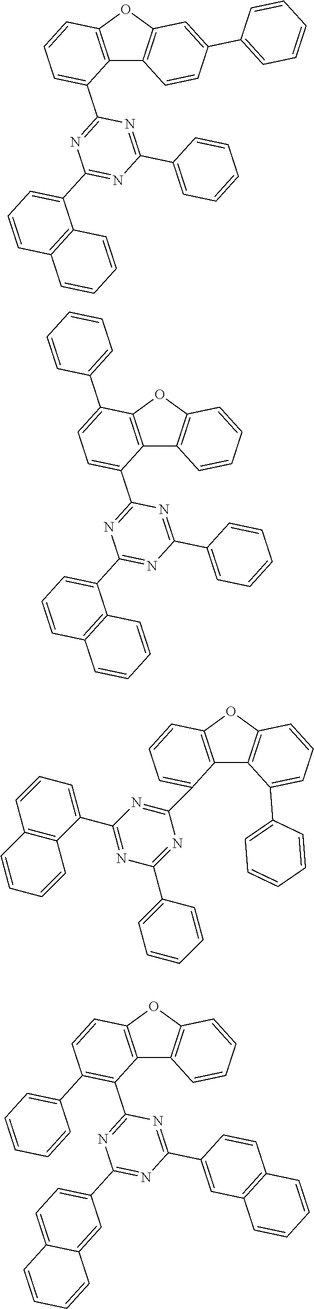

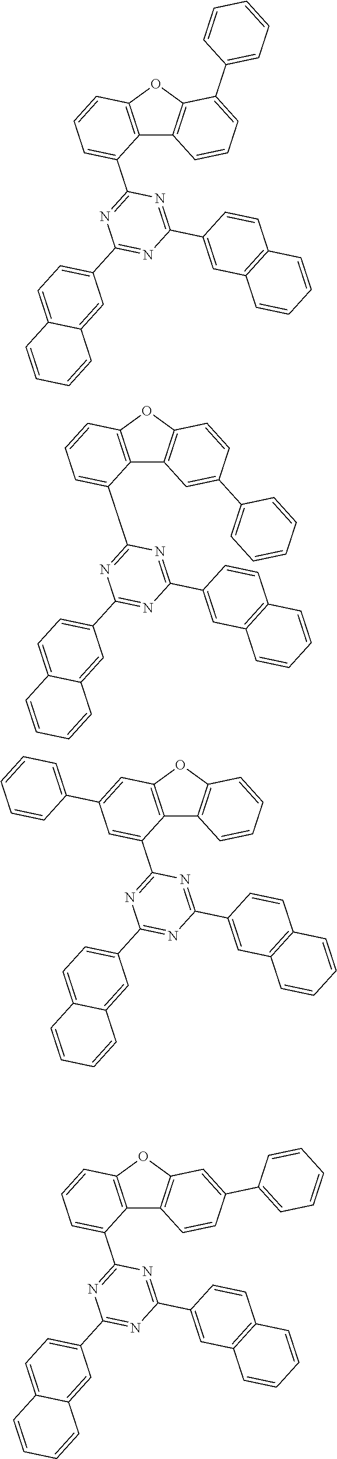

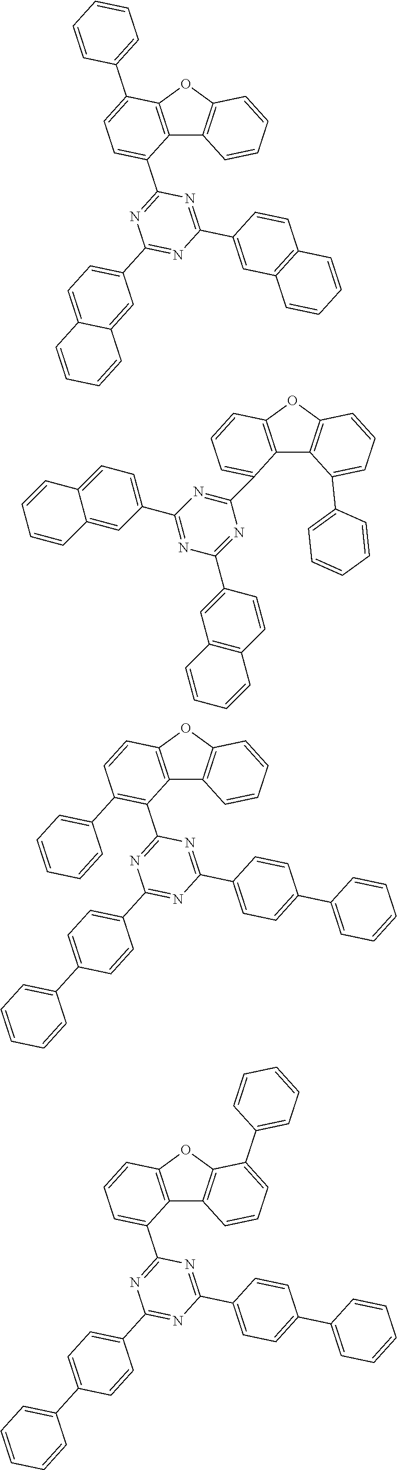

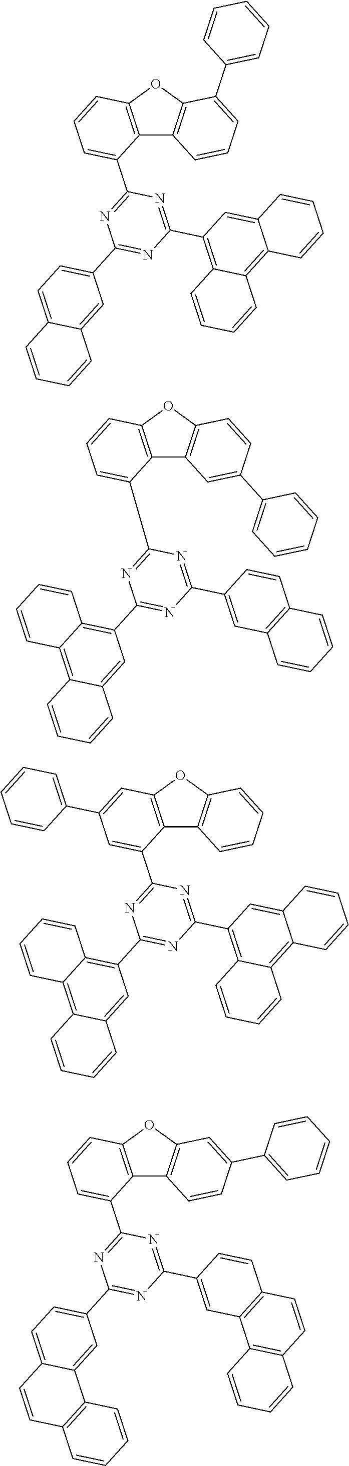

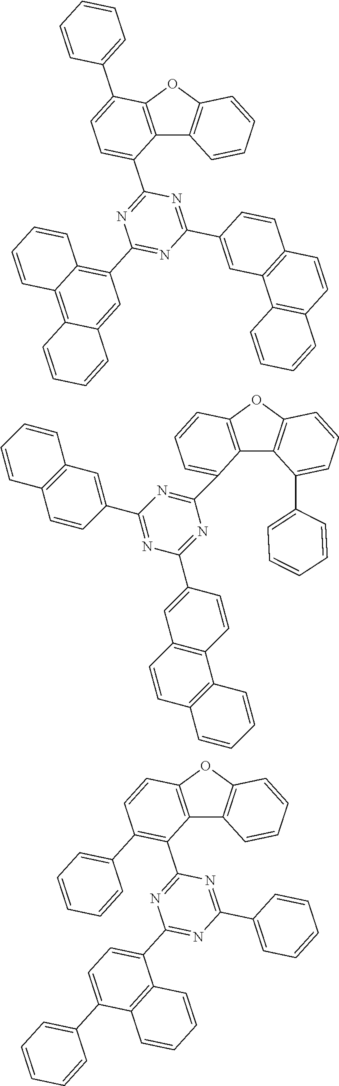

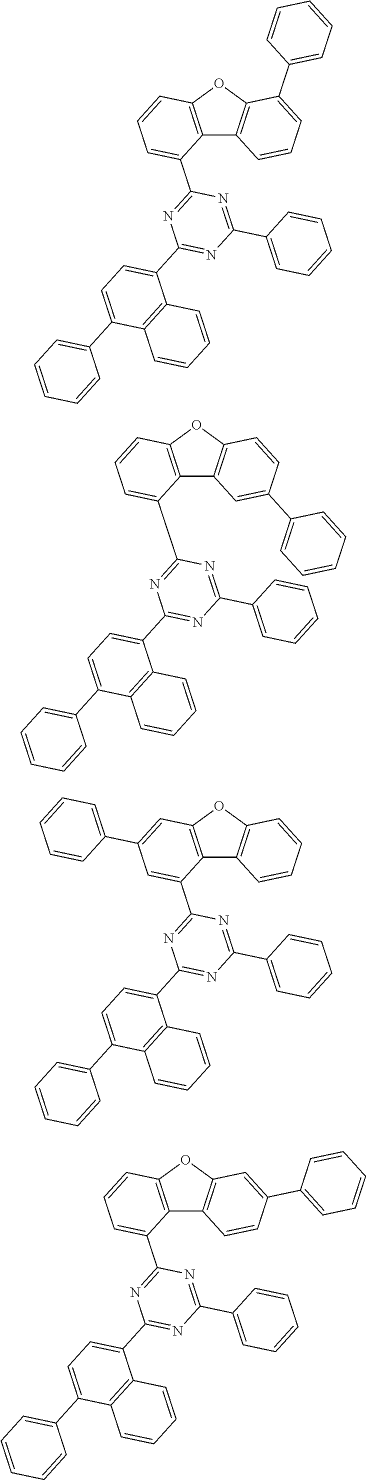

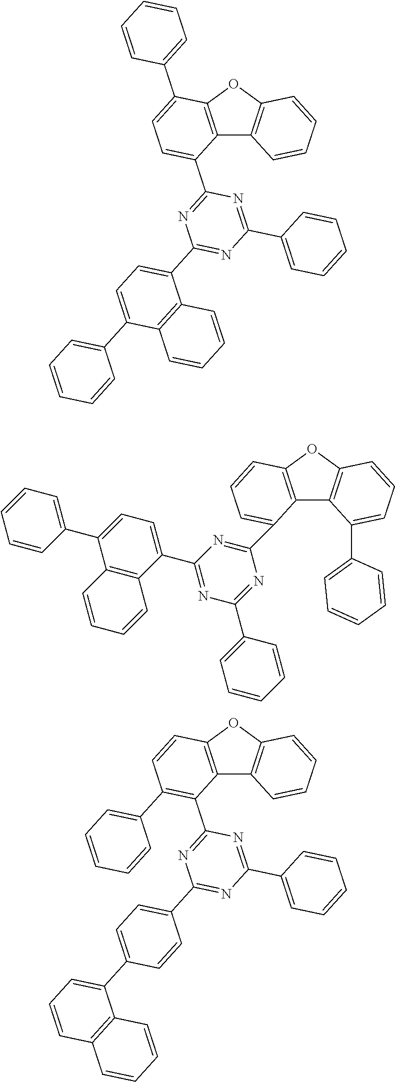

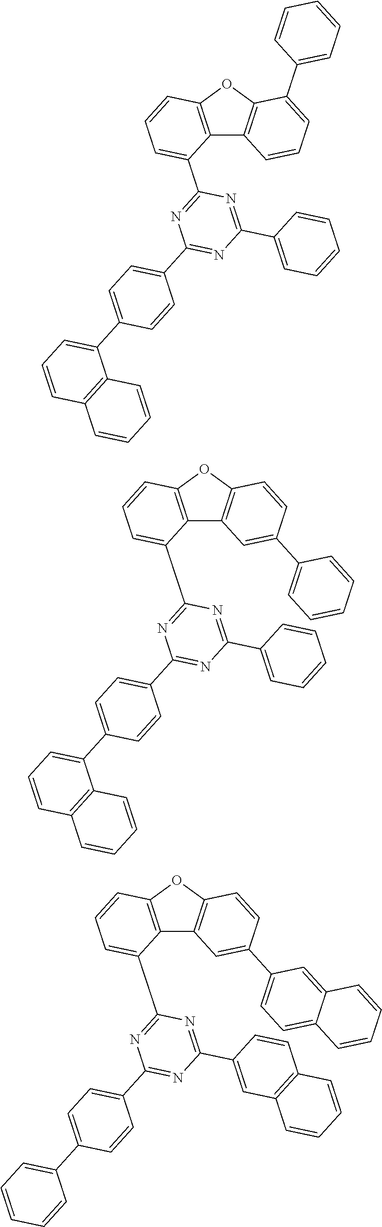

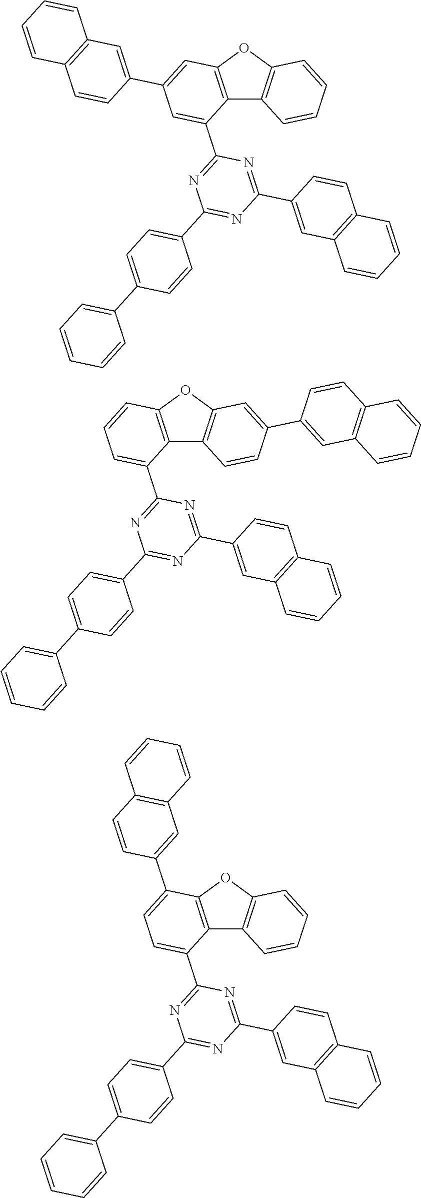

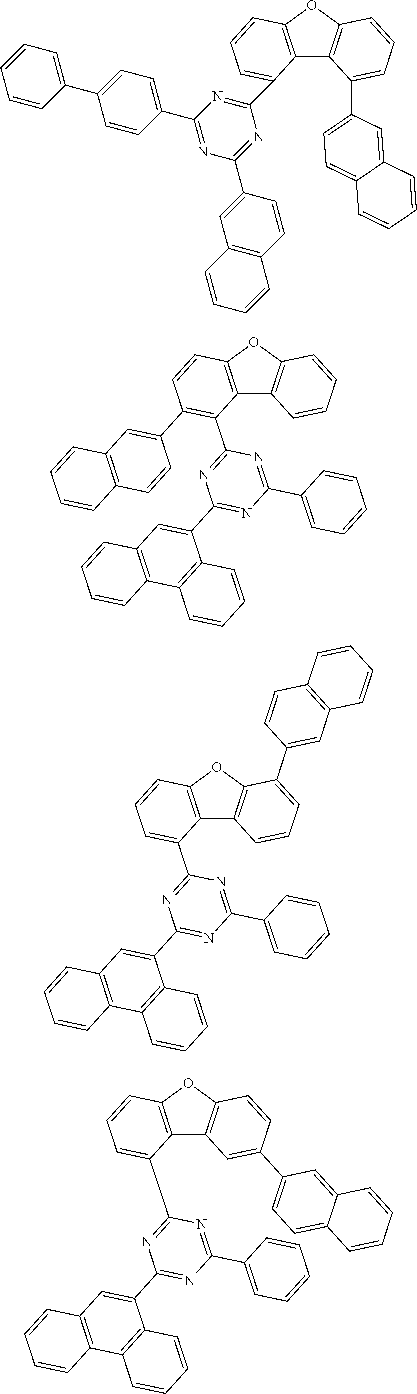

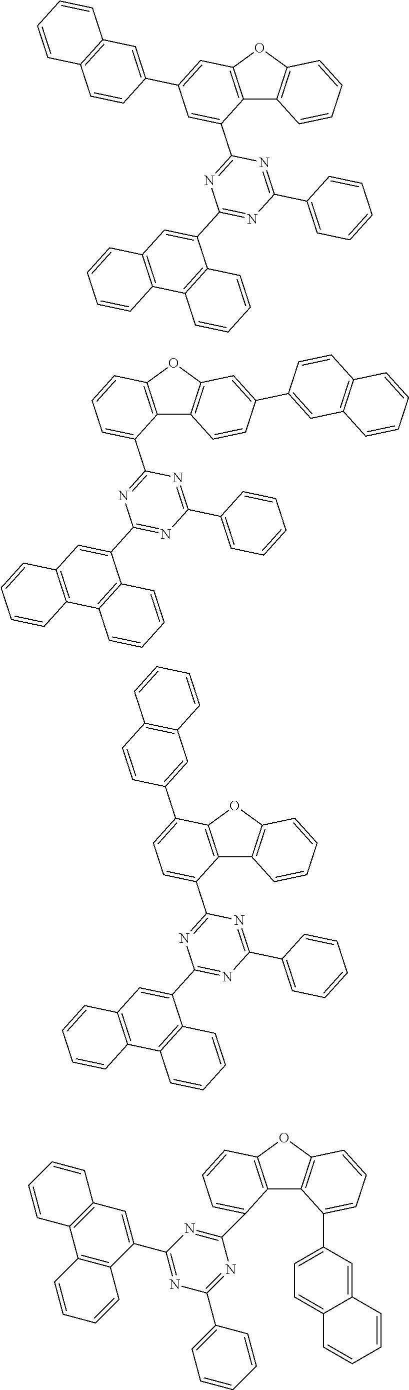

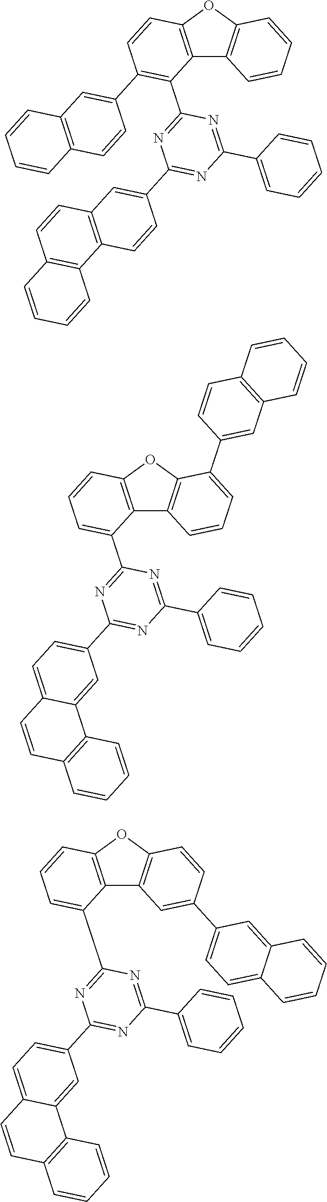

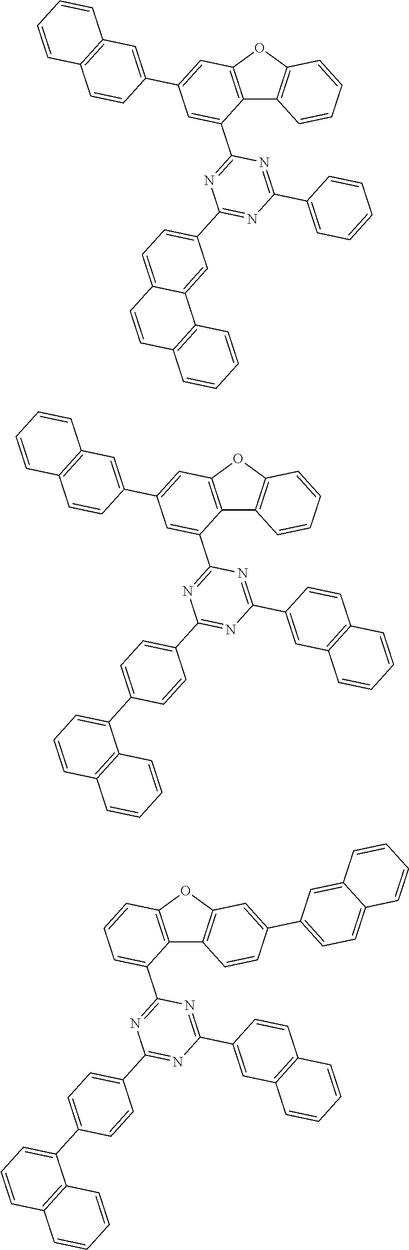 [SMILES: c1ccc(-c2nc(-c3ccc4ccc5ccccc5c4c3)nc(-c3cc(-c4ccc5ccccc5c4)cc4oc5ccccc5c34)n2)cc1 ; c1ccc2cc(-c3cc(-c4nc(-c5ccc(-c6cccc7ccccc67)cc5)nc(-c5ccc6ccccc6c5)n4)c4c(c3)oc3ccccc34)ccc2c1 ; c1ccc2cc(-c3ccc4c(c3)oc3cccc(-c5nc(-c6ccc(-c7cccc8ccccc78)cc6)nc(-c6ccc7ccccc7c6)n5)c34)ccc2c1]

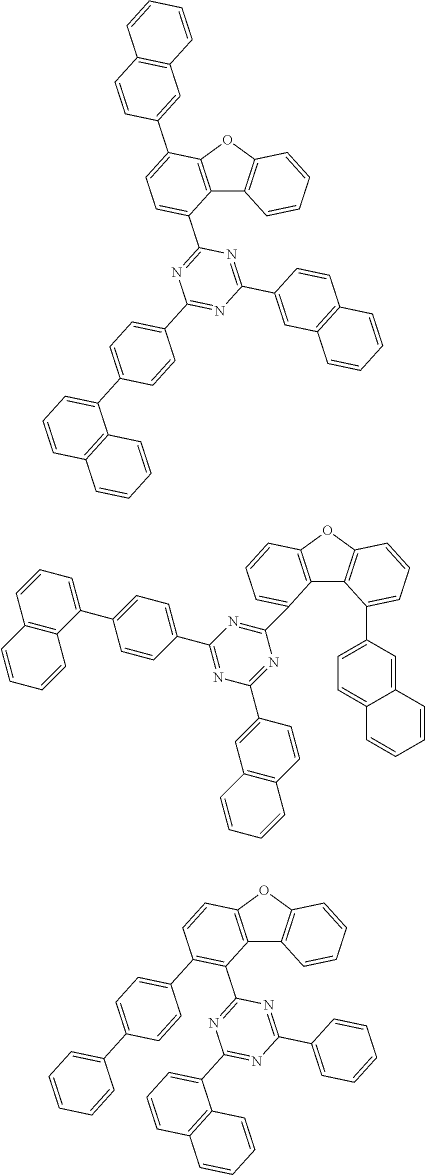 [SMILES: c1ccc2cc(-c3nc(-c4ccc(-c5cccc6ccccc56)cc4)nc(-c4ccc(-c5ccc6ccccc6c5)c5oc6ccccc6c45)n3)ccc2c1 ; c1ccc2cc(-c3nc(-c4ccc(-c5cccc6ccccc56)cc4)nc(-c4cccc5oc6cccc(-c7ccc8ccccc8c7)c6c45)n3)ccc2c1 ; c1ccc(-c2ccc(-c3ccc4oc5ccccc5c4c3-c3nc(-c4ccccc4)nc(-c4cccc5ccccc45)n3)cc2)cc1]

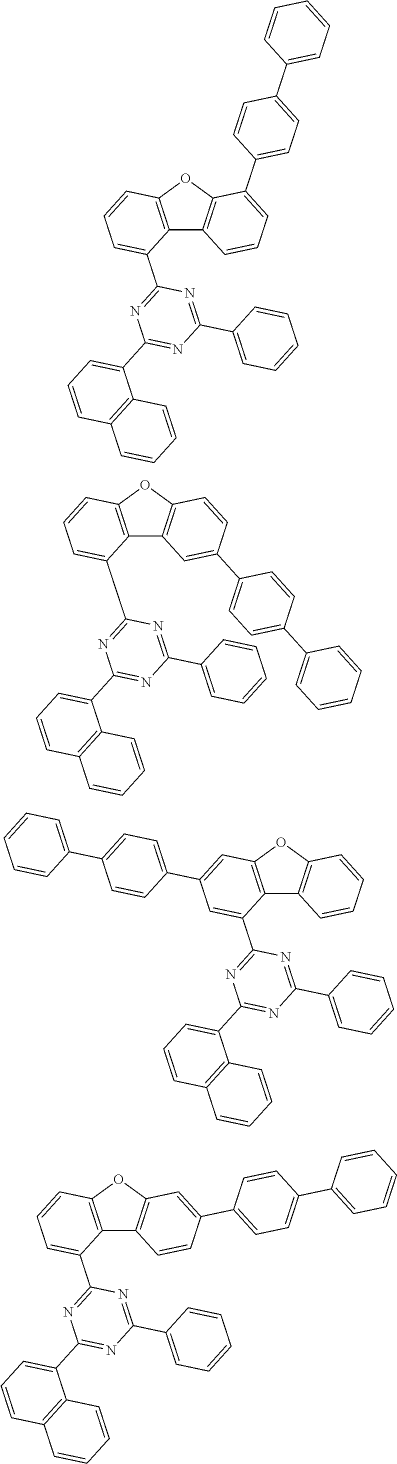

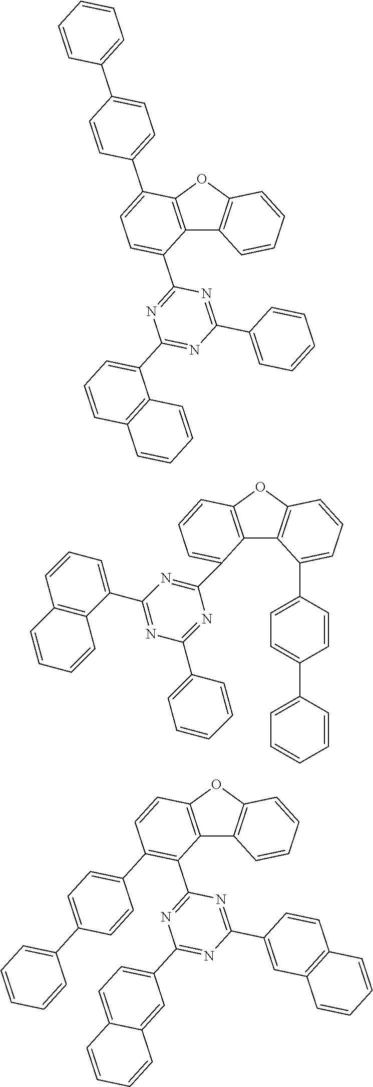

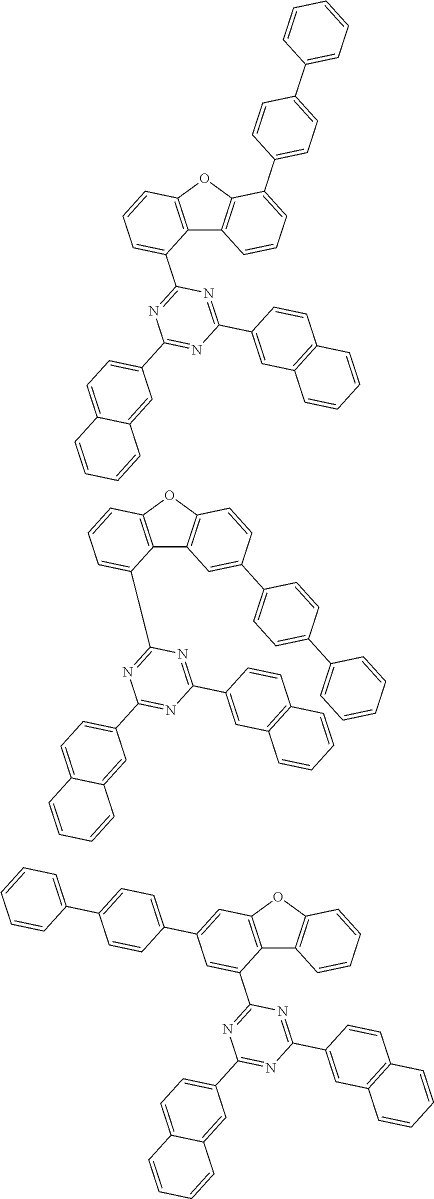

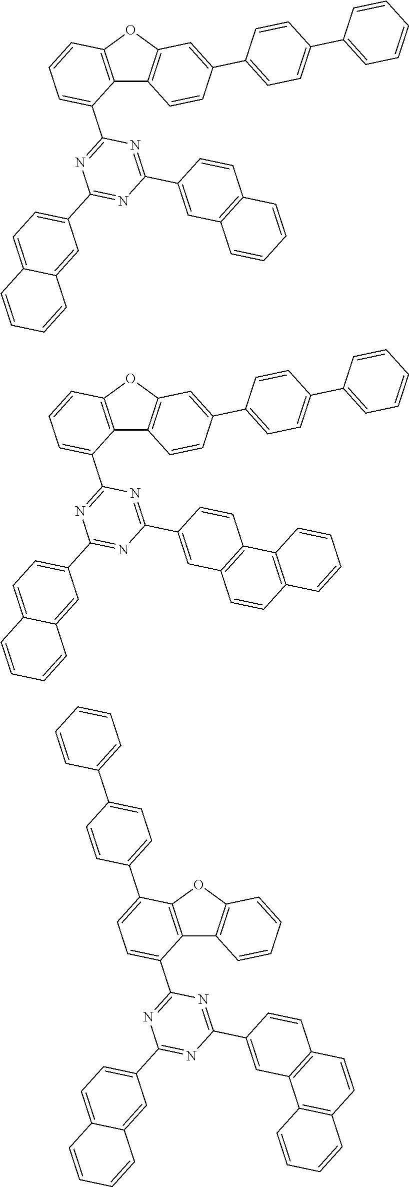

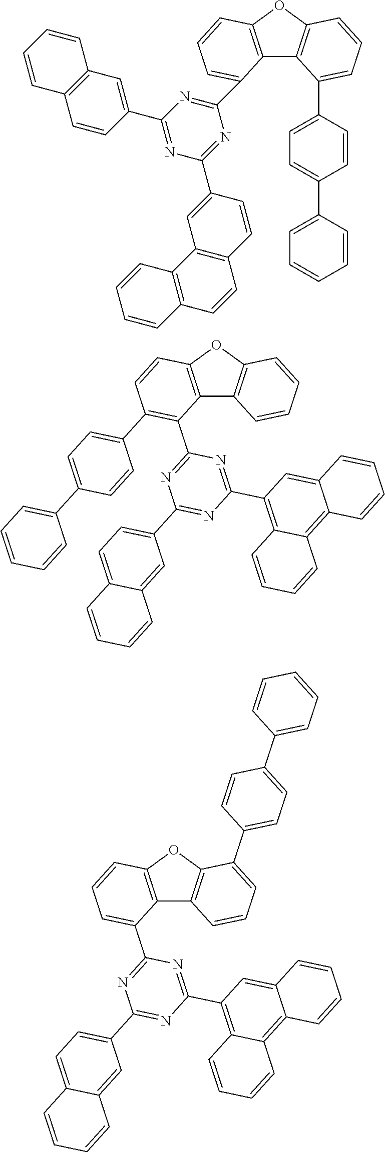

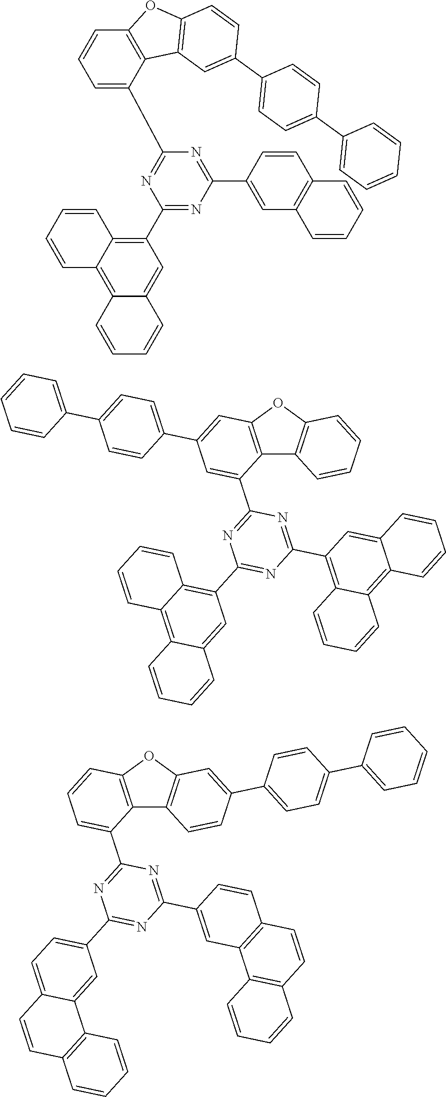

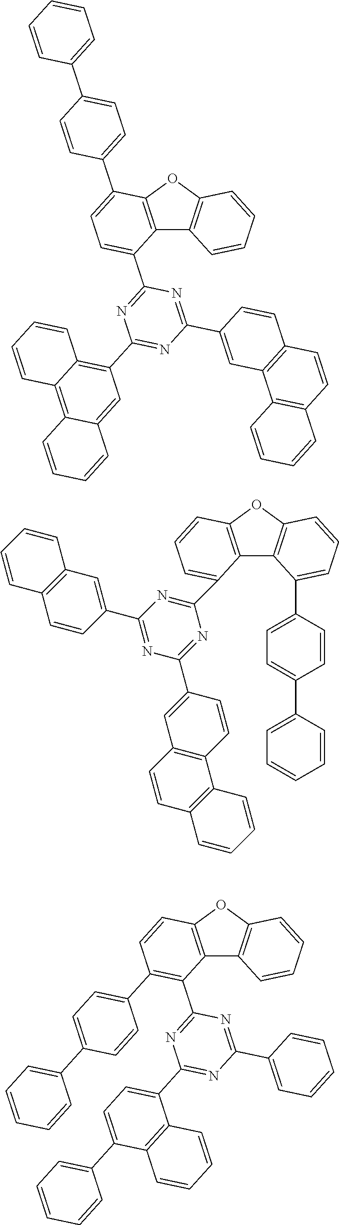

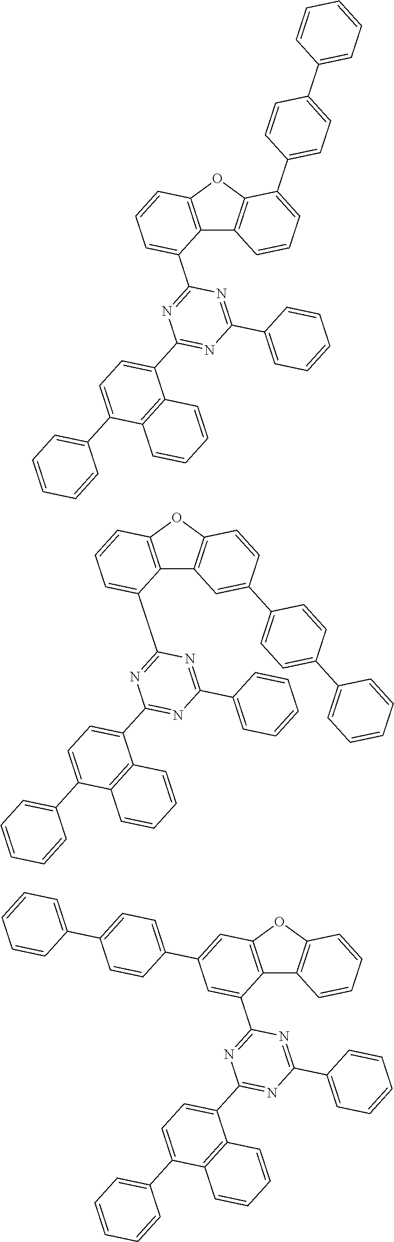

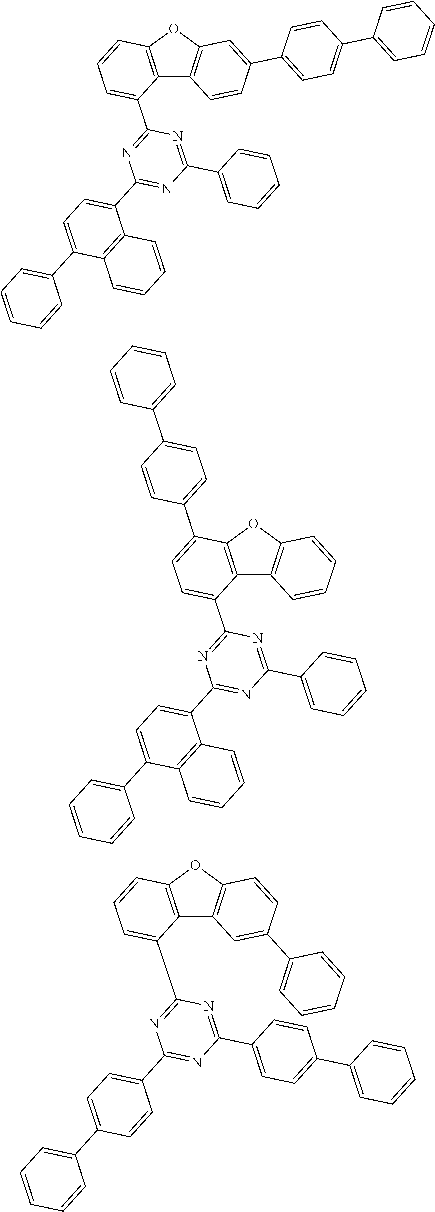

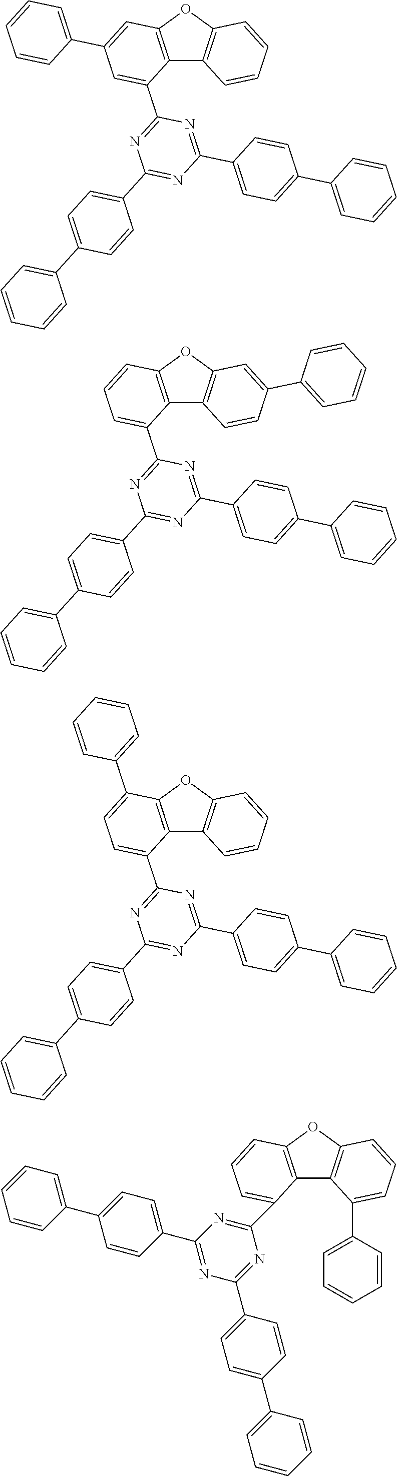 [SMILES: c1ccc(-c2ccc(-c3nc(-c4ccc(-c5ccccc5)cc4)nc(-c4cc(-c5ccccc5)cc5oc6ccccc6c45)n3)cc2)cc1 ; c1ccc(-c2ccc(-c3nc(-c4ccc(-c5ccccc5)cc4)nc(-c4cccc5oc6cc(-c7ccccc7)ccc6c45)n3)cc2)cc1 ; c1ccc(-c2ccc(-c3nc(-c4ccc(-c5ccccc5)cc4)nc(-c4ccc(-c5ccccc5)c5oc6ccccc6c45)n3)cc2)cc1 ; c1ccc(-c2ccc(-c3nc(-c4ccc(-c5ccccc5)cc4)nc(-c4cccc5oc6cccc(-c7ccccc7)c6c45)n3)cc2)cc1]

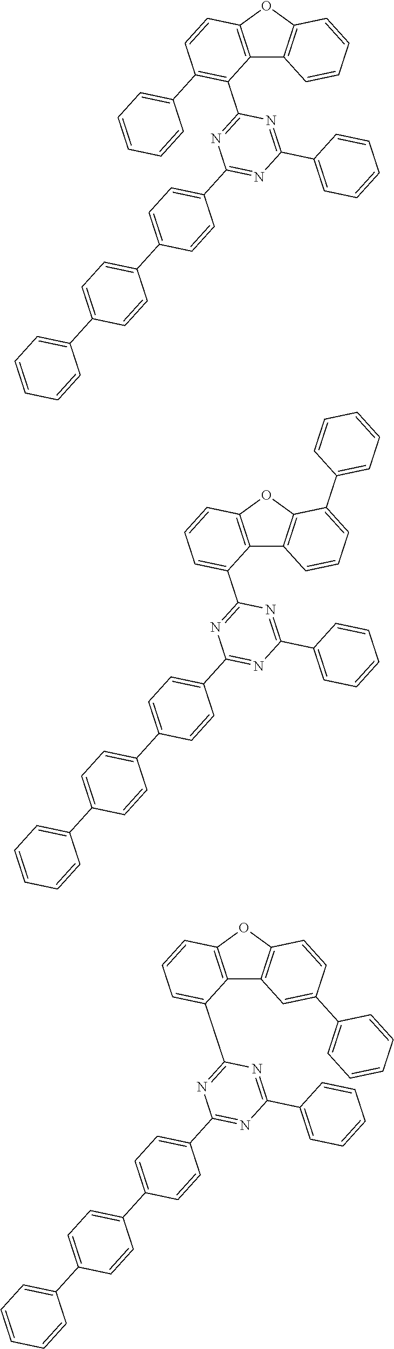 [SMILES: c1ccc(-c2ccc(-c3ccc(-c4nc(-c5ccccc5)nc(-c5c(-c6ccccc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccccc8)cccc7c56)n4)cc3)cc2)cc1 ; c1ccc(-c2ccc(-c3ccc(-c4nc(-c5ccccc5)nc(-c5cccc6oc7ccc(-c8ccccc8)cc7c56)n4)cc3)cc2)cc1]

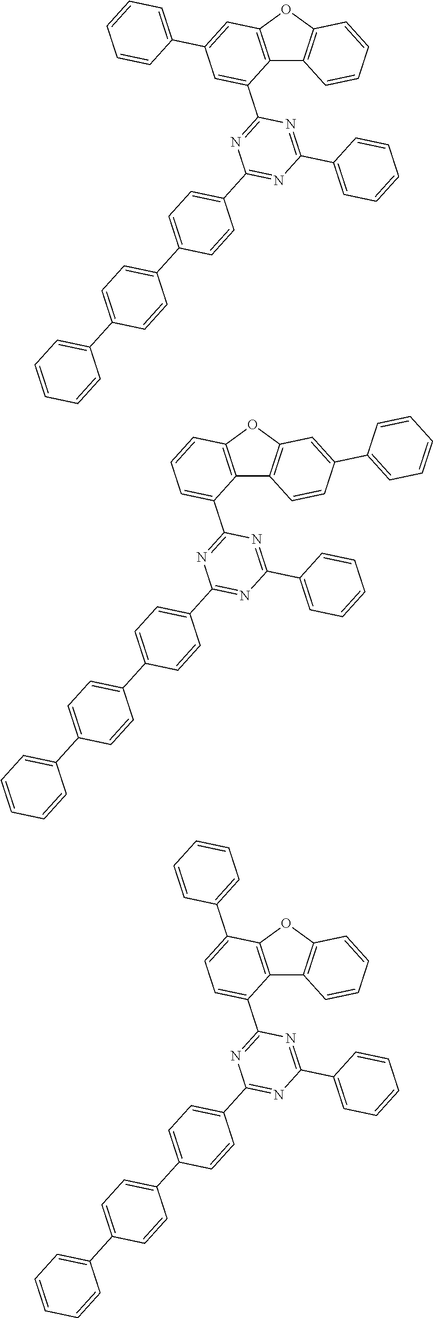 [SMILES: c1ccc(-c2ccc(-c3ccc(-c4nc(-c5ccccc5)nc(-c5cc(-c6ccccc6)cc6oc7ccccc7c56)n4)cc3)cc2)cc1 ; c1ccc(-c2ccc(-c3ccc(-c4nc(-c5ccccc5)nc(-c5cccc6oc7cc(-c8ccccc8)ccc7c56)n4)cc3)cc2)cc1 ; c1ccc(-c2ccc(-c3ccc(-c4nc(-c5ccccc5)nc(-c5ccc(-c6ccccc6)c6oc7ccccc7c56)n4)cc3)cc2)cc1]

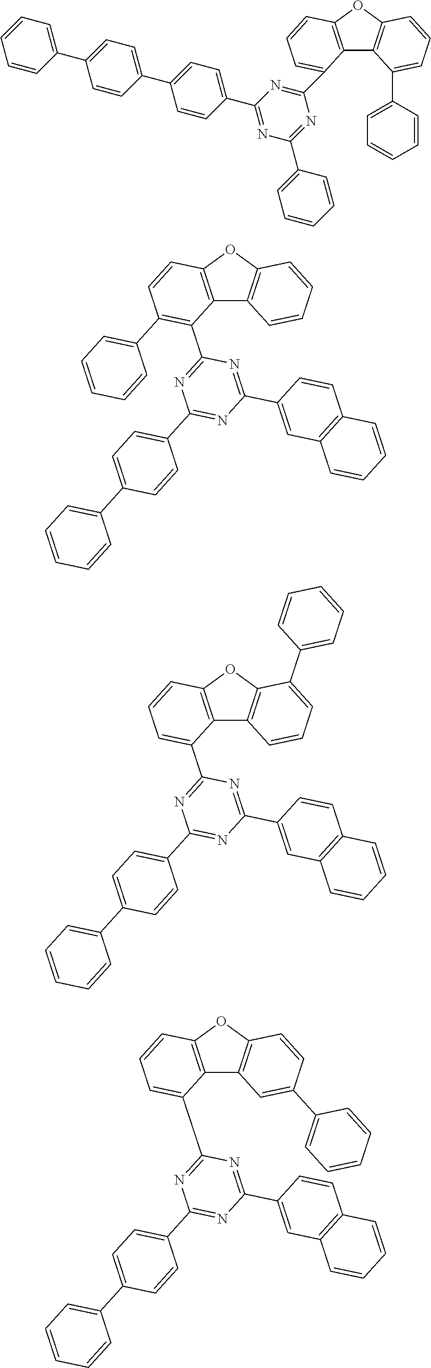

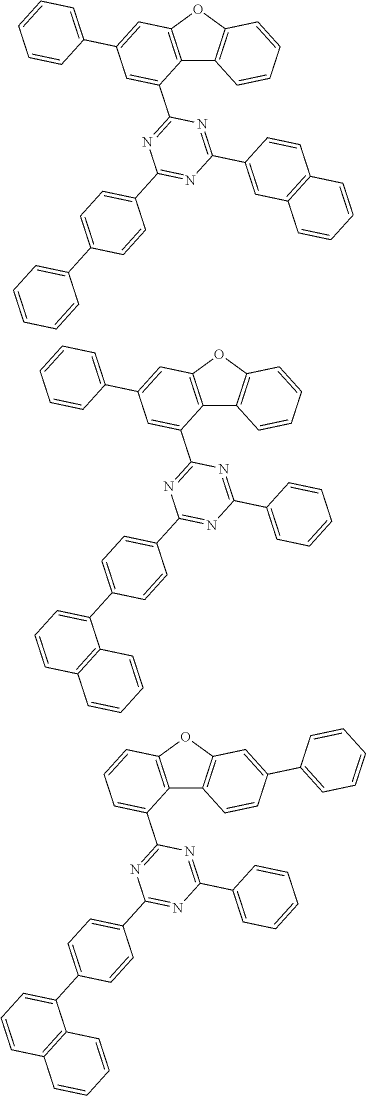

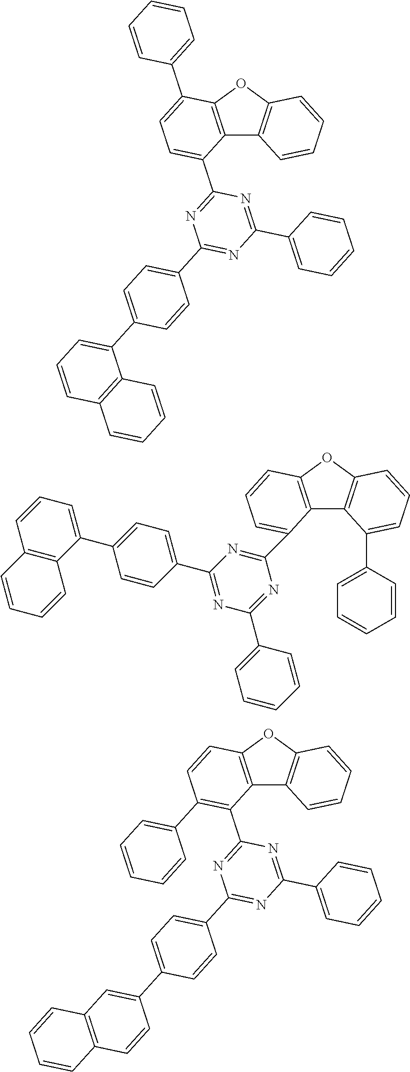

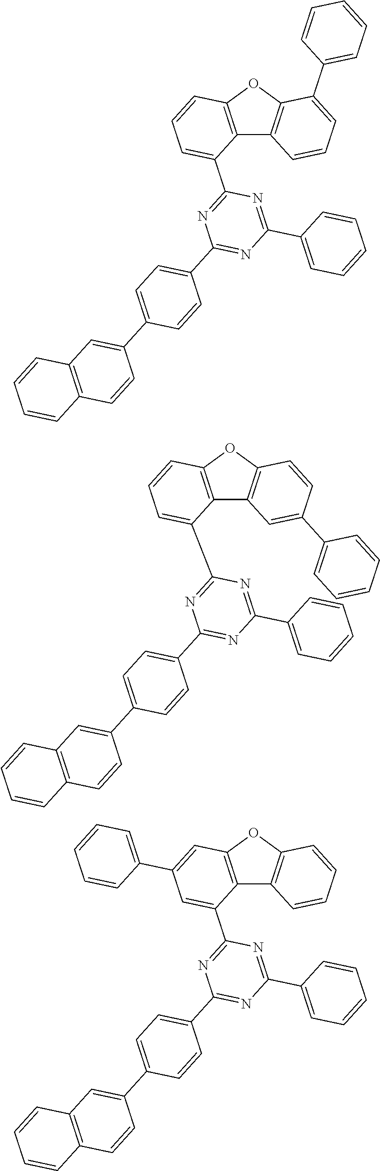

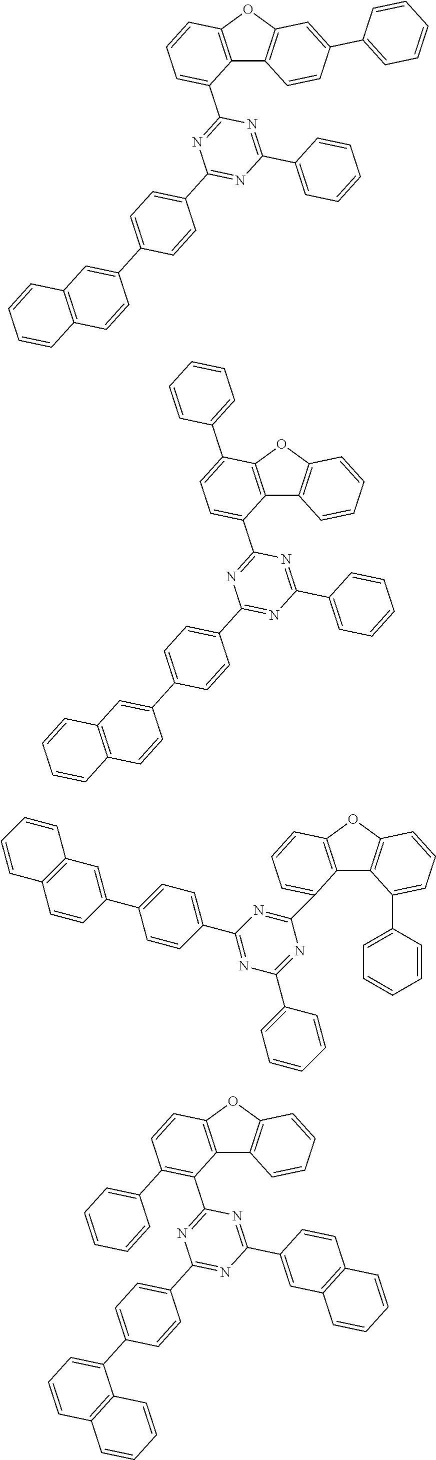

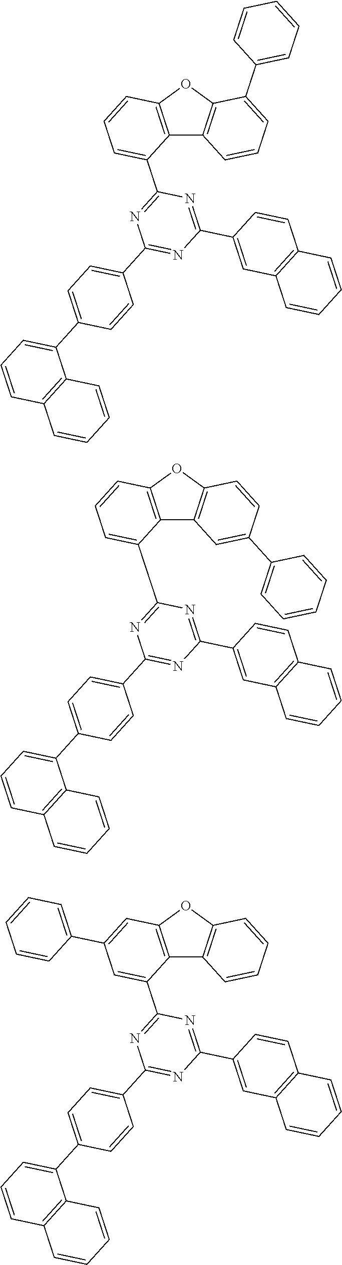 [SMILES: c1ccc(-c2cccc3c2oc2cccc(-c4nc(-c5ccc(-c6cccc7ccccc67)cc5)nc(-c5ccc6ccccc6c5)n4)c23)cc1 ; c1ccc(-c2ccc3oc4cccc(-c5nc(-c6ccc(-c7cccc8ccccc78)cc6)nc(-c6ccc7ccccc7c6)n5)c4c3c2)cc1 ; c1ccc(-c2cc(-c3nc(-c4ccc(-c5cccc6ccccc56)cc4)nc(-c4ccc5ccccc5c4)n3)c3c(c2)oc2ccccc23)cc1]

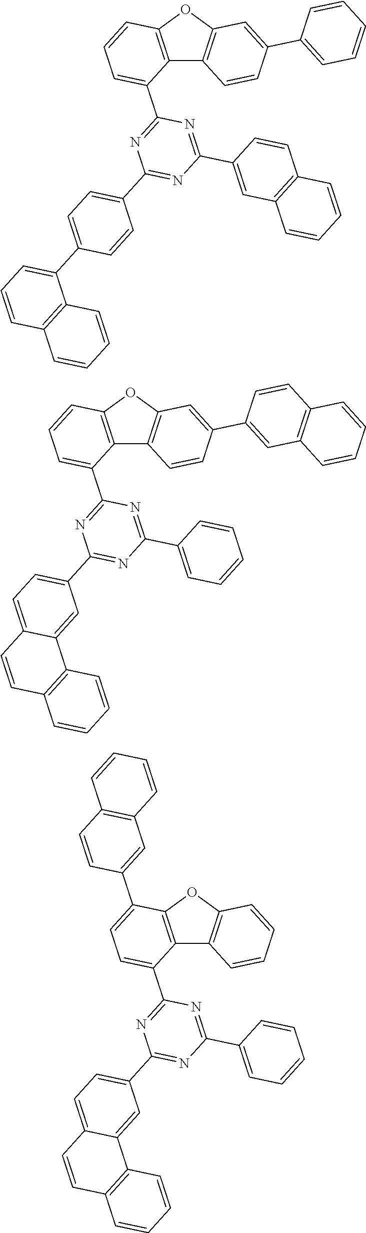

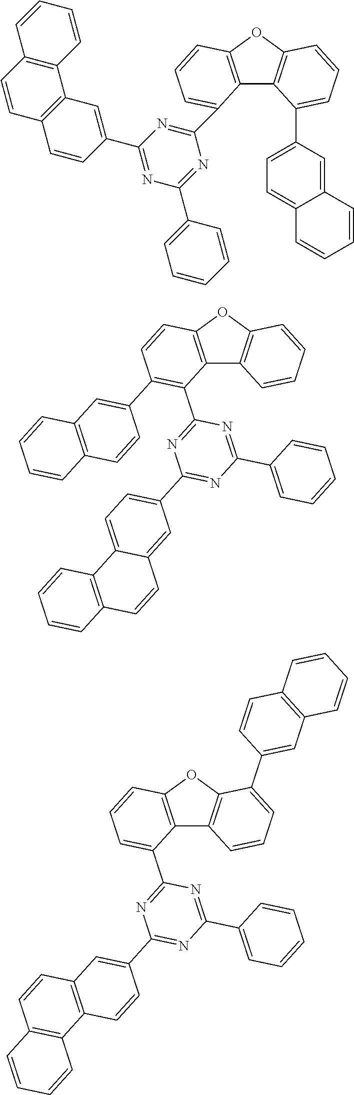

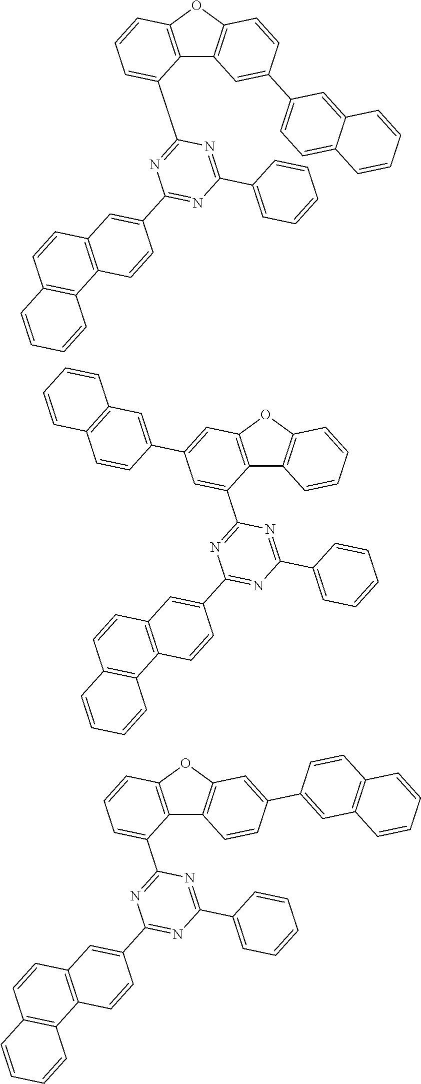

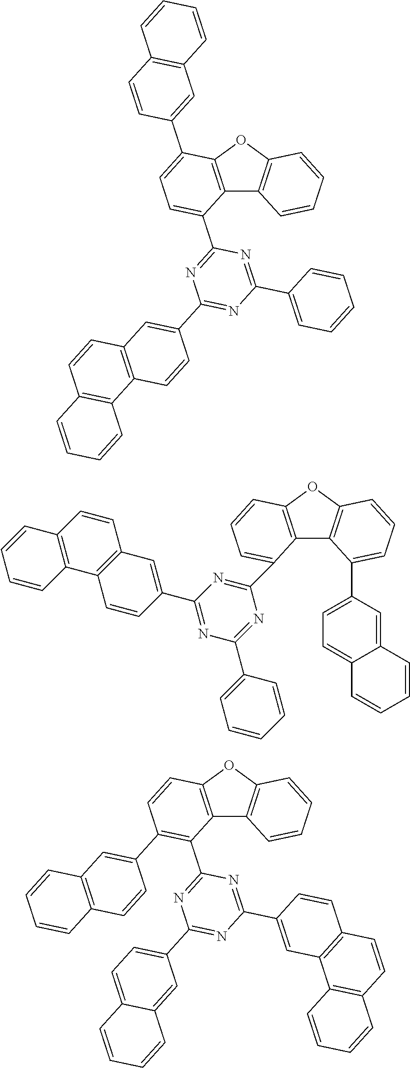

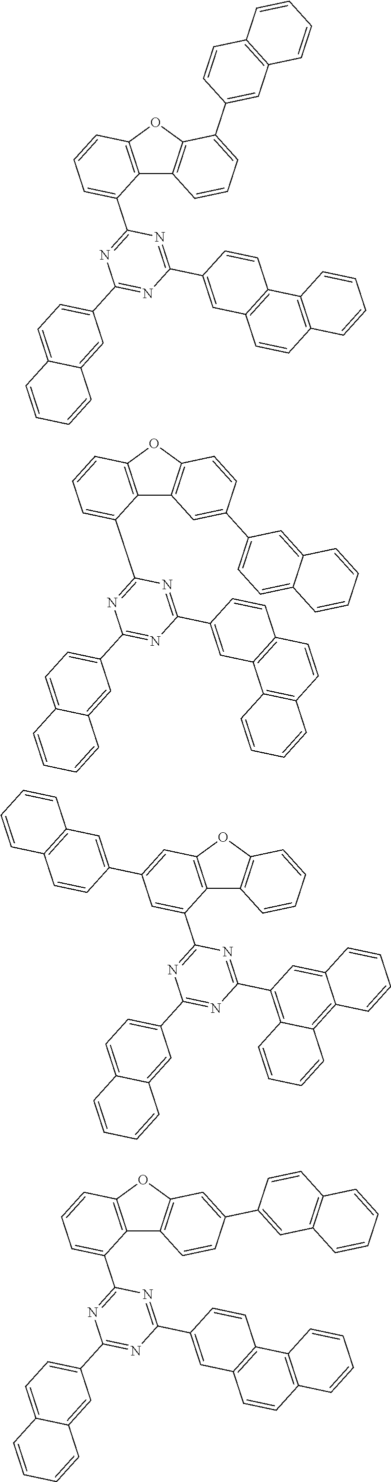

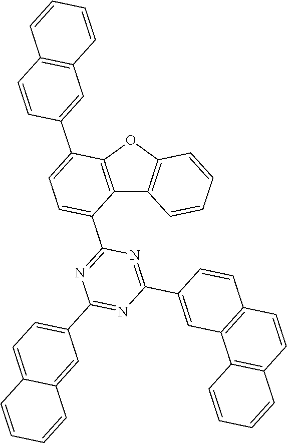 [SMILES: c1ccc2cc(-c3nc(-c4ccc5ccc6ccccc6c5c4)nc(-c4ccc(-c5ccc6ccccc6c5)c5oc6ccccc6c45)n3)ccc2c1]

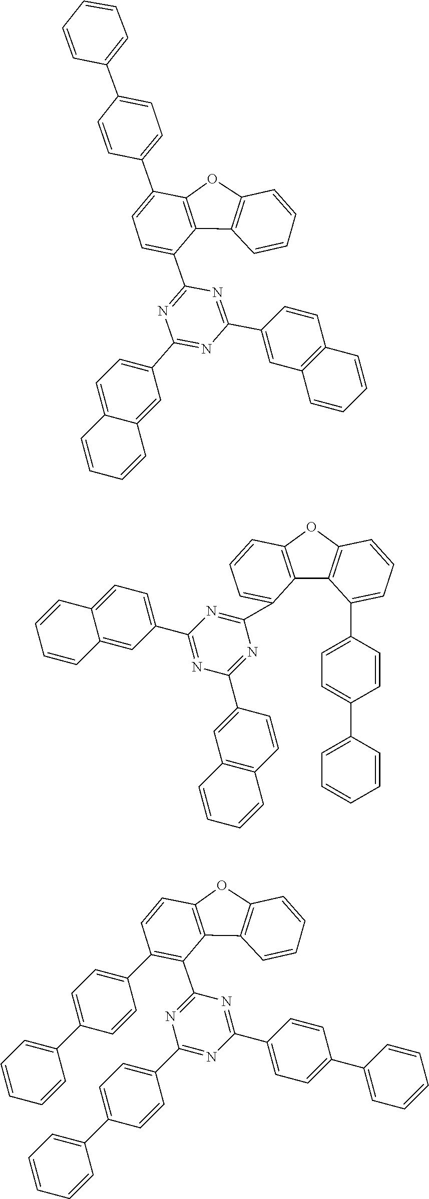 [SMILES: c1ccc(-c2ccc(-c3ccc(-c4nc(-c5ccc6ccccc6c5)nc(-c5ccc6ccccc6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccccc8c7)n6)c5c34)cc2)cc1 ; c1ccc(-c2ccc(-c3nc(-c4ccc(-c5ccccc5)cc4)nc(-c4c(-c5ccc(-c6ccccc6)cc5)ccc5oc6ccccc6c45)n3)cc2)cc1]

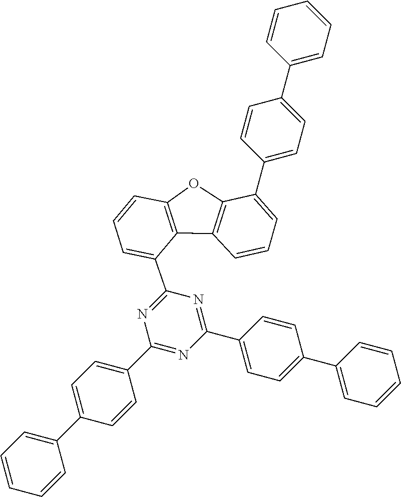 [SMILES: c1ccc(-c2ccc(-c3nc(-c4ccc(-c5ccccc5)cc4)nc(-c4cccc5oc6c(-c7ccc(-c8ccccc8)cc7)cccc6c45)n3)cc2)cc1]

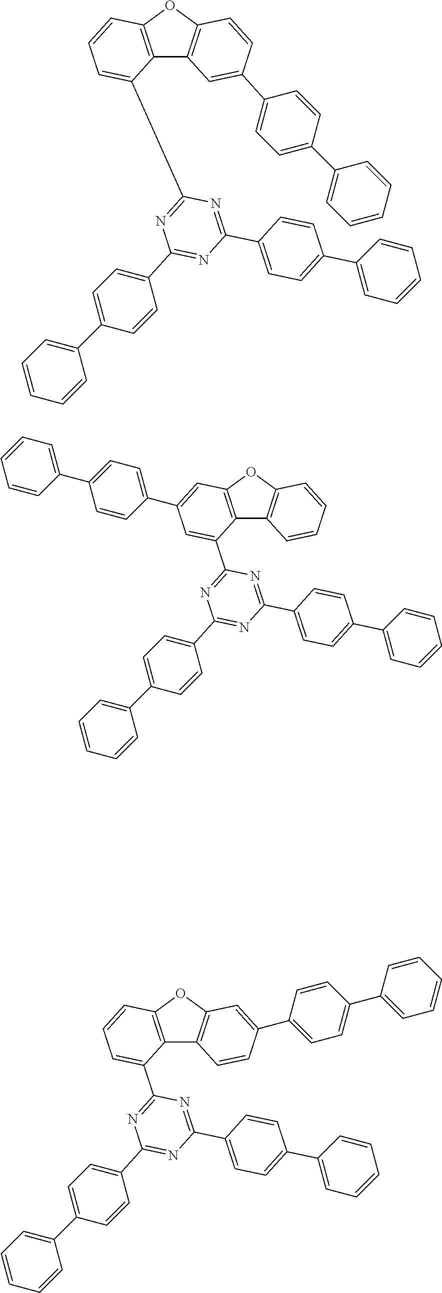

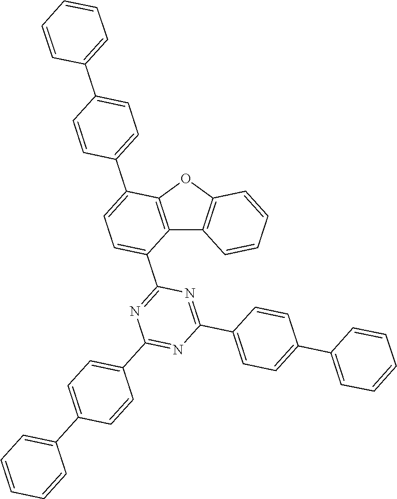 [SMILES: c1ccc(-c2ccc(-c3nc(-c4ccc(-c5ccccc5)cc4)nc(-c4ccc(-c5ccc(-c6ccccc6)cc5)c5oc6ccccc6c45)n3)cc2)cc1]

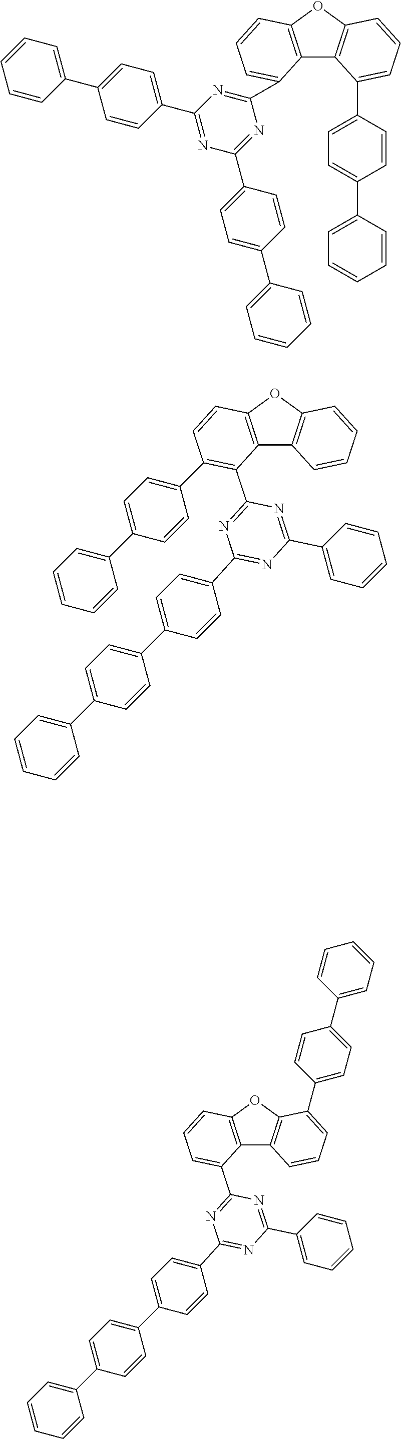 [SMILES: c1ccc(-c2ccc(-c3nc(-c4ccc(-c5ccccc5)cc4)nc(-c4cccc5oc6cccc(-c7ccc(-c8ccccc8)cc7)c6c45)n3)cc2)cc1 ; c1ccc(-c2ccc(-c3ccc(-c4nc(-c5ccccc5)nc(-c5c(-c6ccc(-c7ccccc7)cc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccc(-c9ccccc9)cc8)cccc7c56)n4)cc3)cc2)cc1]

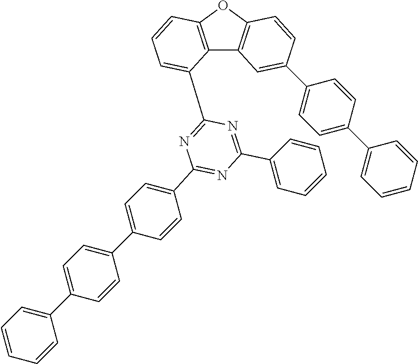 [SMILES: c1ccc(-c2ccc(-c3ccc(-c4nc(-c5ccccc5)nc(-c5cccc6oc7ccc(-c8ccc(-c9ccccc9)cc8)cc7c56)n4)cc3)cc2)cc1]

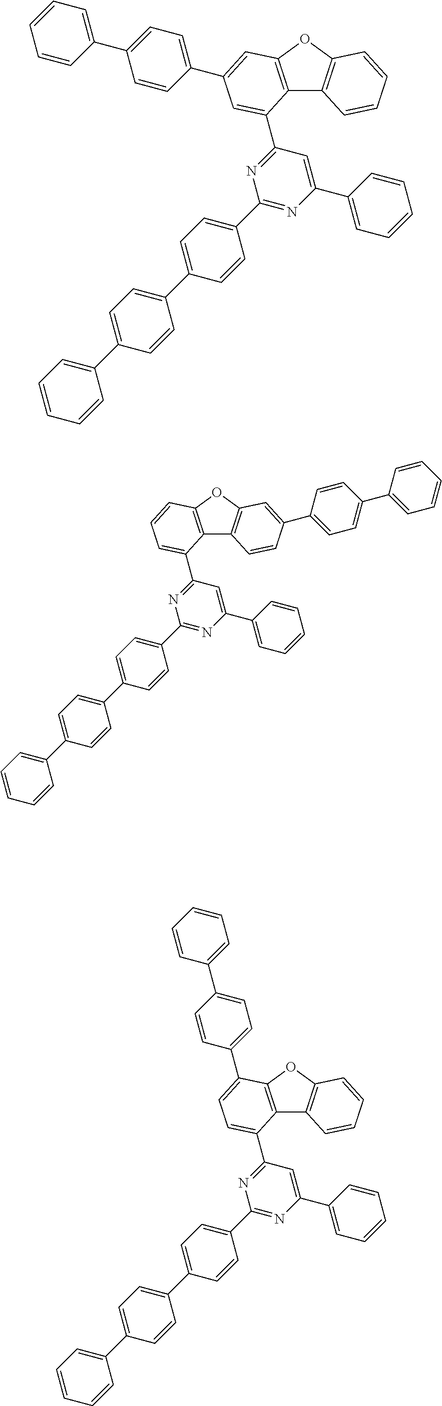 [SMILES: c1ccc(-c2ccc(-c3ccc(-c4nc(-c5ccccc5)cc(-c5cc(-c6ccc(-c7ccccc7)cc6)cc6oc7ccccc7c56)n4)cc3)cc2)cc1 ; c1ccc(-c2ccc(-c3ccc(-c4nc(-c5ccccc5)cc(-c5cccc6oc7cc(-c8ccc(-c9ccccc9)cc8)ccc7c56)n4)cc3)cc2)cc1 ; c1ccc(-c2ccc(-c3ccc(-c4nc(-c5ccccc5)cc(-c5ccc(-c6ccc(-c7ccccc7)cc6)c6oc7ccccc7c56)n4)cc3)cc2)cc1]

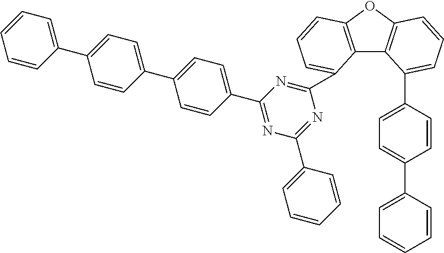 [SMILES: c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1]

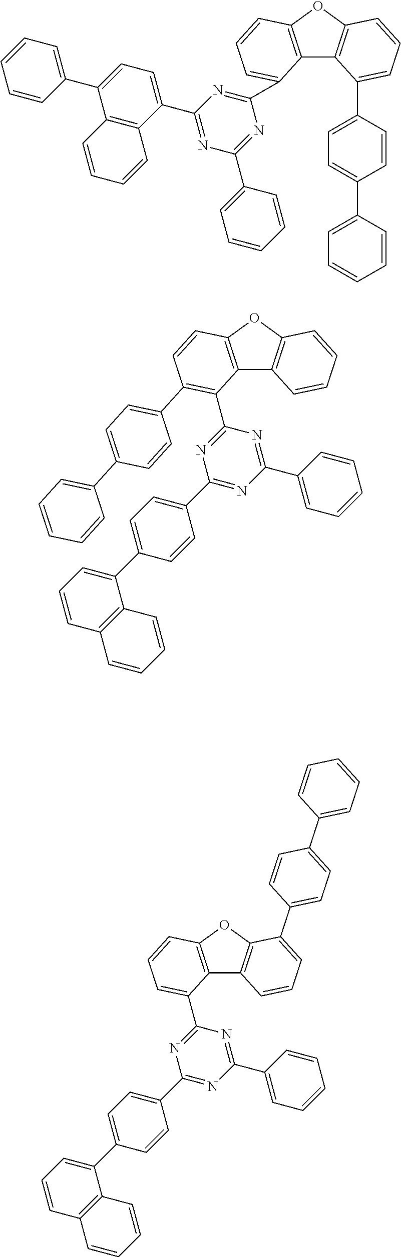 [SMILES: c1ccc(-c2ccc(-c3cccc4oc5cccc(-c6nc(-c7ccccc7)nc(-c7ccc(-c8ccccc8)c8ccccc78)n6)c5c34)cc2)cc1 ; c1ccc(-c2ccc(-c3ccc4oc5ccccc5c4c3-c3nc(-c4ccccc4)nc(-c4ccc(-c5cccc6ccccc56)cc4)n3)cc2)cc1 ; c1ccc(-c2ccc(-c3cccc4c3oc3cccc(-c5nc(-c6ccccc6)nc(-c6ccc(-c7cccc8ccccc78)cc6)n5)c34)cc2)cc1]

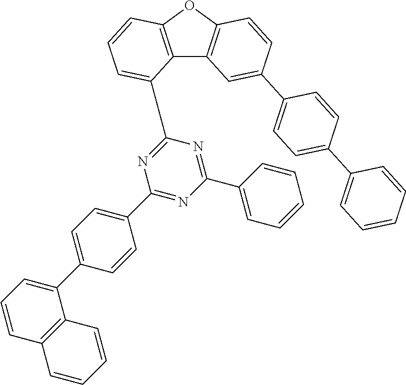 [SMILES: c1ccc(-c2ccc(-c3ccc4oc5cccc(-c6nc(-c7ccccc7)nc(-c7ccc(-c8cccc9ccccc89)cc7)n6)c5c4c3)cc2)cc1]

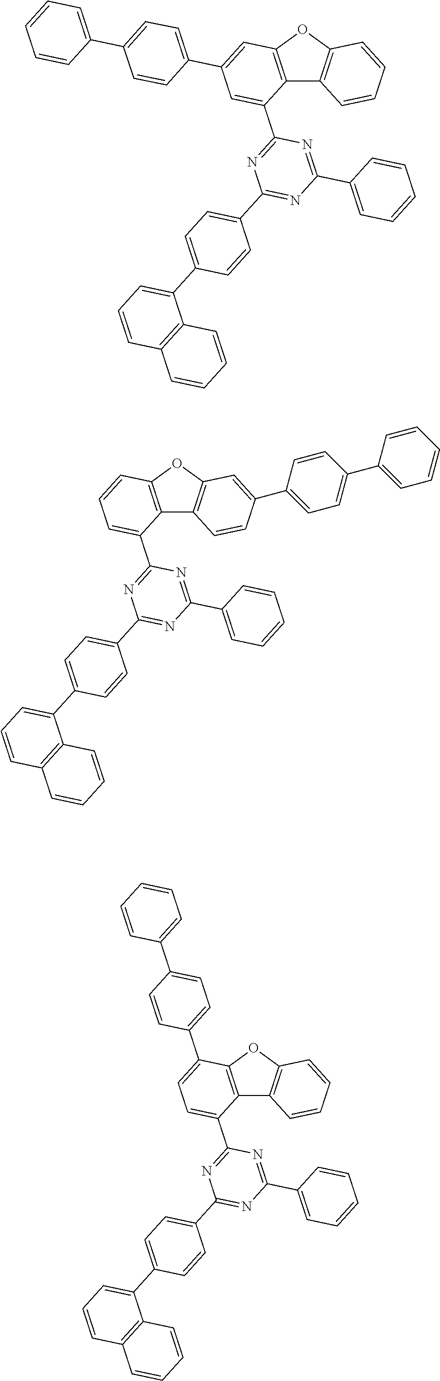 [SMILES: c1ccc(-c2ccc(-c3cc(-c4nc(-c5ccccc5)nc(-c5ccc(-c6cccc7ccccc67)cc5)n4)c4c(c3)oc3ccccc34)cc2)cc1 ; c1ccc(-c2ccc(-c3ccc4c(c3)oc3cccc(-c5nc(-c6ccccc6)nc(-c6ccc(-c7cccc8ccccc78)cc6)n5)c34)cc2)cc1 ; c1ccc(-c2ccc(-c3ccc(-c4nc(-c5ccccc5)nc(-c5ccc(-c6cccc7ccccc67)cc5)n4)c4c3oc3ccccc34)cc2)cc1]

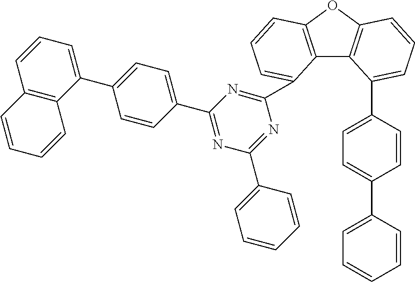 [SMILES: c1ccc(-c2ccc(-c3cccc4oc5cccc(-c6nc(-c7ccccc7)nc(-c7ccc(-c8cccc9ccccc89)cc7)n6)c5c34)cc2)cc1]

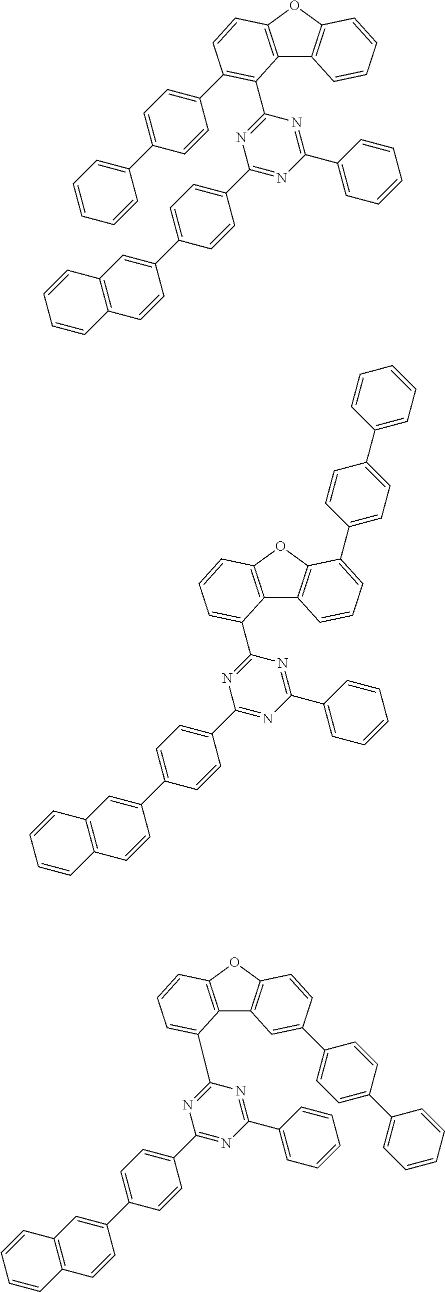 [SMILES: c1ccc(-c2ccc(-c3ccc4oc5ccccc5c4c3-c3nc(-c4ccccc4)nc(-c4ccc(-c5ccc6ccccc6c5)cc4)n3)cc2)cc1 ; c1ccc(-c2ccc(-c3cccc4c3oc3cccc(-c5nc(-c6ccccc6)nc(-c6ccc(-c7ccc8ccccc8c7)cc6)n5)c34)cc2)cc1 ; c1ccc(-c2ccc(-c3ccc4oc5cccc(-c6nc(-c7ccccc7)nc(-c7ccc(-c8ccc9ccccc9c8)cc7)n6)c5c4c3)cc2)cc1]

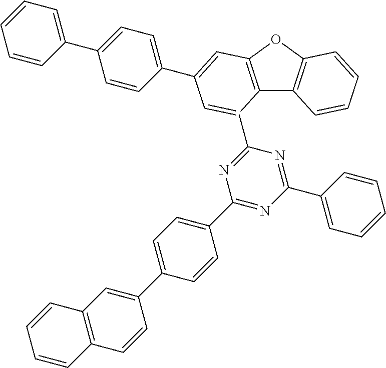

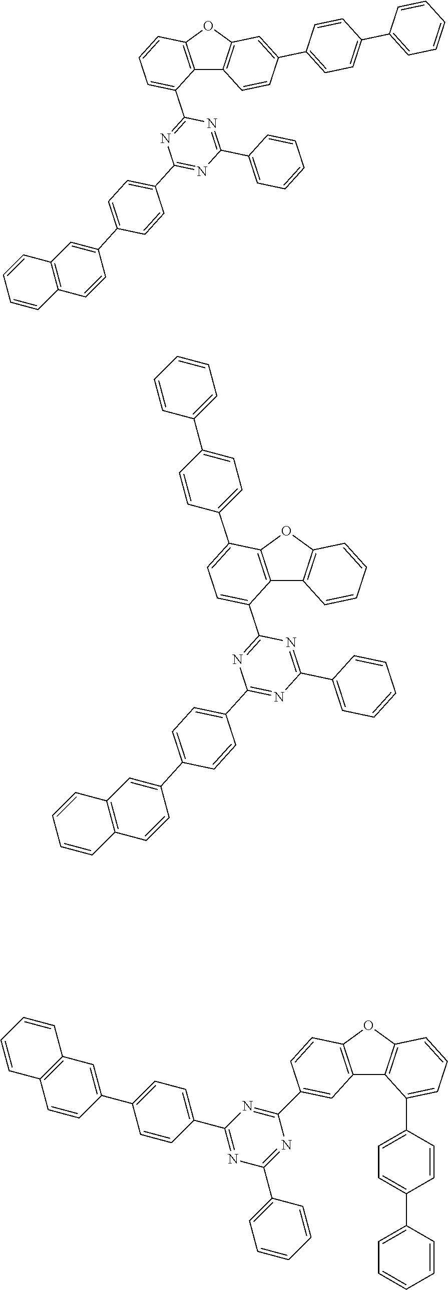 [SMILES: c1ccc(-c2ccc(-c3ccc4c(c3)oc3cccc(-c5nc(-c6ccccc6)nc(-c6ccc(-c7ccc8ccccc8c7)cc6)n5)c34)cc2)cc1 ; c1ccc(-c2ccc(-c3ccc(-c4nc(-c5ccccc5)nc(-c5ccc(-c6ccc7ccccc7c6)cc5)n4)c4c3oc3ccccc34)cc2)cc1 ; c1ccc(-c2ccc(-c3cccc4oc5ccc(-c6nc(-c7ccccc7)nc(-c7ccc(-c8ccc9ccccc9c8)cc7)n6)cc5c34)cc2)cc1]

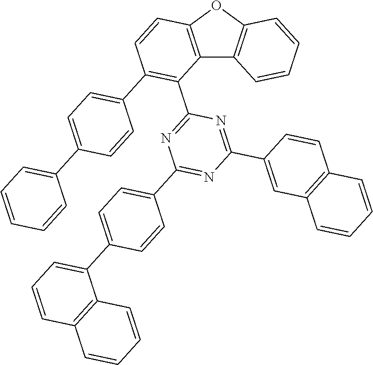 [SMILES: c1ccc(-c2ccc(-c3ccc4oc5ccccc5c4c3-c3nc(-c4ccc(-c5cccc6ccccc56)cc4)nc(-c4ccc5ccccc5c4)n3)cc2)cc1]

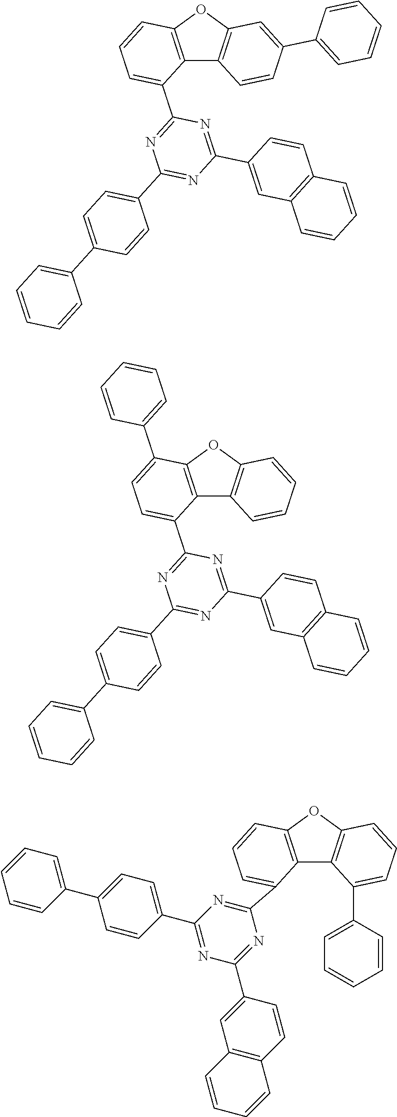 [SMILES: c1ccc(-c2ccc(-c3nc(-c4ccc5ccccc5c4)nc(-c4cccc5oc6cc(-c7ccccc7)ccc6c45)n3)cc2)cc1 ; c1ccc(-c2ccc(-c3nc(-c4ccc5ccccc5c4)nc(-c4ccc(-c5ccccc5)c5oc6ccccc6c45)n3)cc2)cc1 ; c1ccc(-c2ccc(-c3nc(-c4ccc5ccccc5c4)nc(-c4cccc5oc6cccc(-c7ccccc7)c6c45)n3)cc2)cc1]

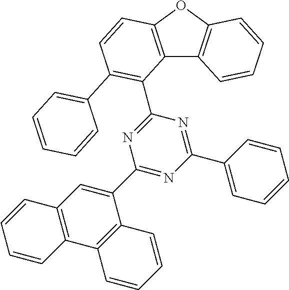 [SMILES: c1ccc(-c2nc(-c3cc4ccccc4c4ccccc34)nc(-c3c(-c4ccccc4)ccc4oc5ccccc5c34)n2)cc1]

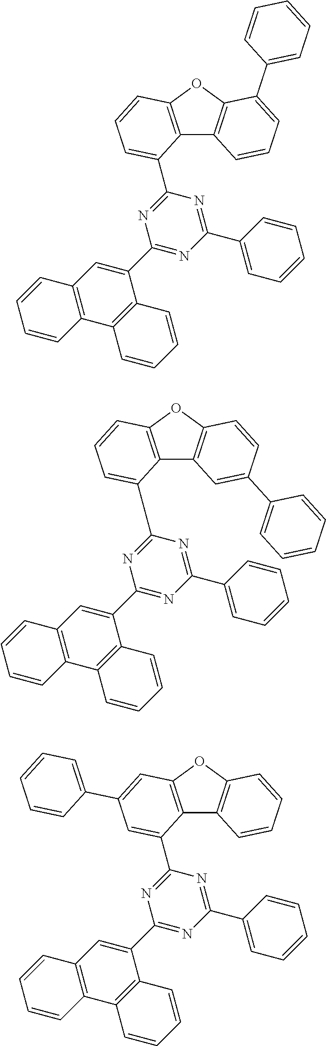

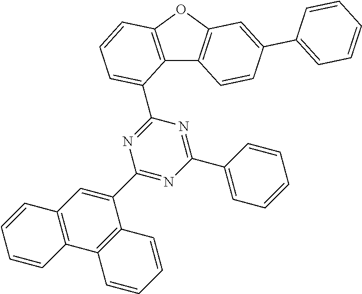 [SMILES: c1ccc(-c2ccc3c(c2)oc2cccc(-c4nc(-c5ccccc5)nc(-c5cc6ccccc6c6ccccc56)n4)c23)cc1]

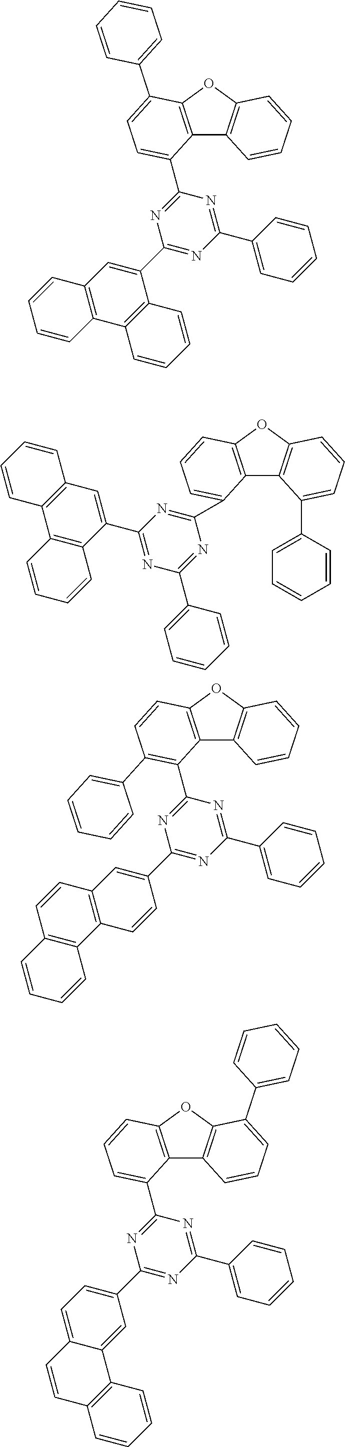

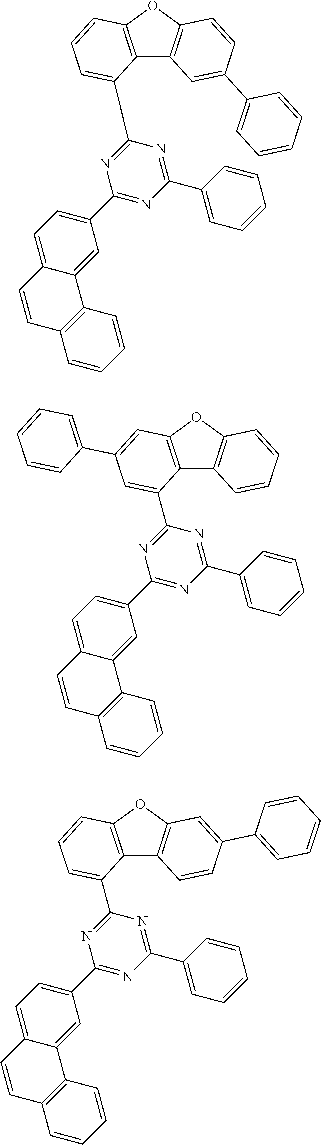 [SMILES: c1ccc(-c2ccc3oc4cccc(-c5nc(-c6ccccc6)nc(-c6ccc7ccc8ccccc8c7c6)n5)c4c3c2)cc1 ; c1ccc(-c2cc(-c3nc(-c4ccccc4)nc(-c4ccc5ccc6ccccc6c5c4)n3)c3c(c2)oc2ccccc23)cc1 ; c1ccc(-c2ccc3c(c2)oc2cccc(-c4nc(-c5ccccc5)nc(-c5ccc6ccc7ccccc7c6c5)n4)c23)cc1]

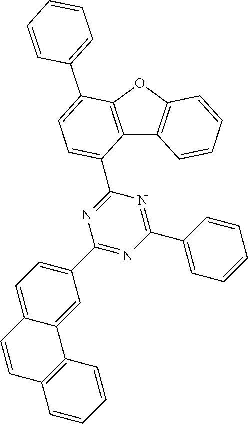 [SMILES: c1ccc(-c2nc(-c3ccc4ccc5ccccc5c4c3)nc(-c3ccc(-c4ccccc4)c4oc5ccccc5c34)n2)cc1]

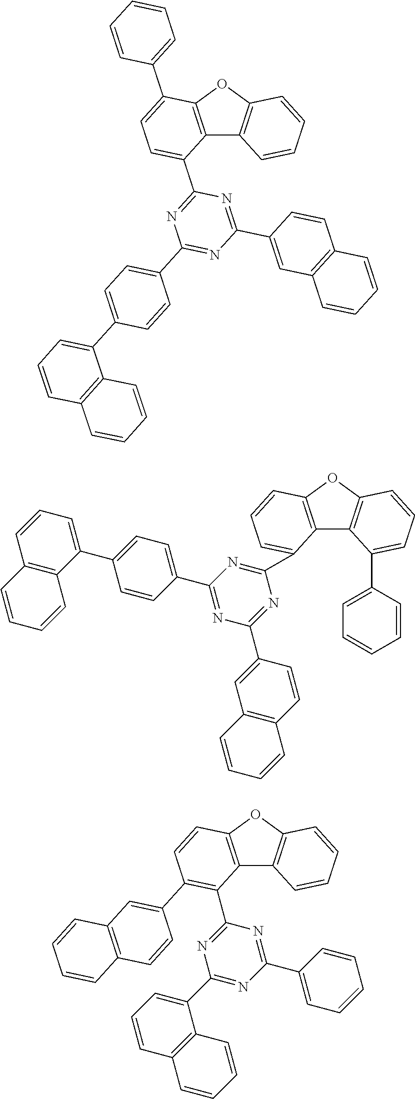

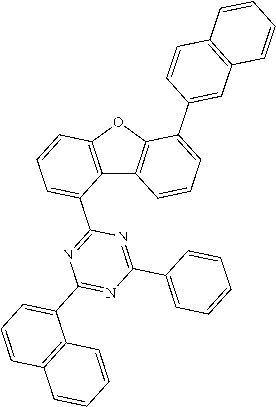 [SMILES: c1ccc(-c2nc(-c3cccc4ccccc34)nc(-c3cccc4oc5c(-c6ccc7ccccc7c6)cccc5c34)n2)cc1]

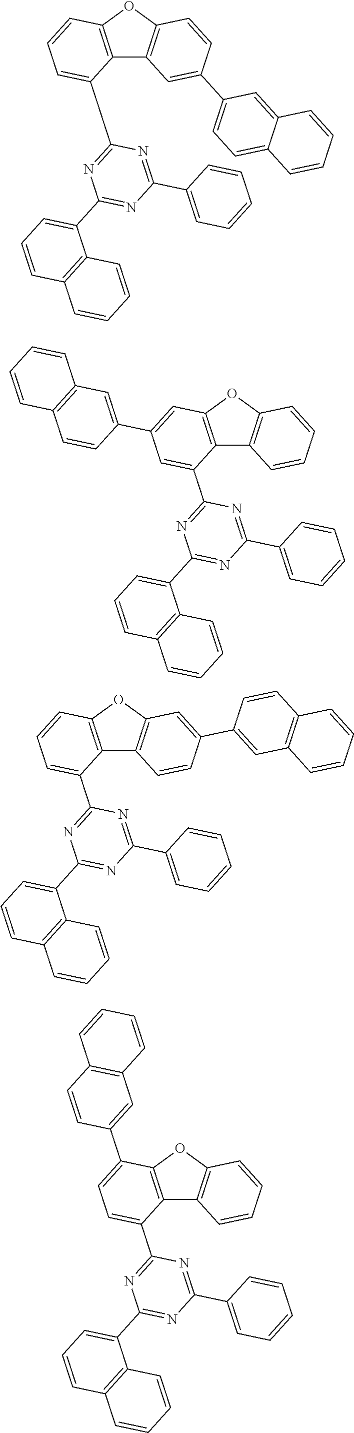

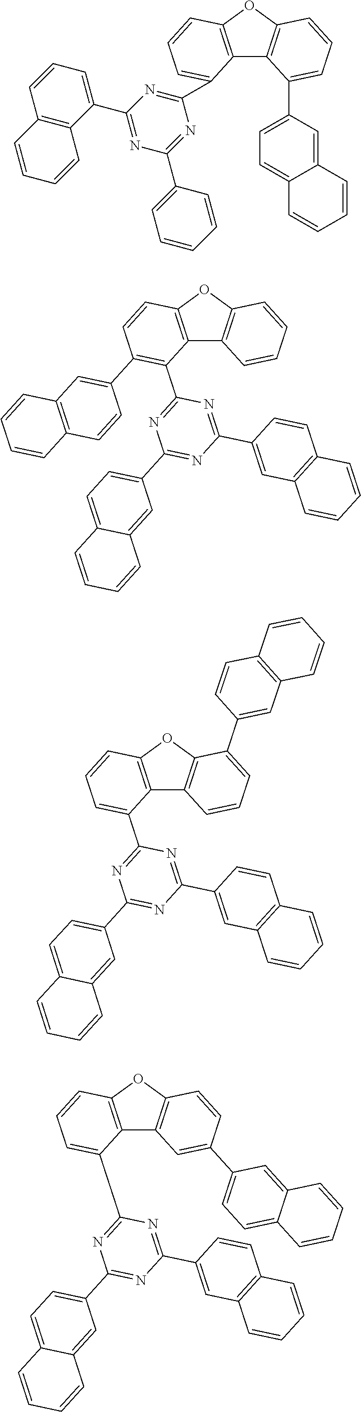

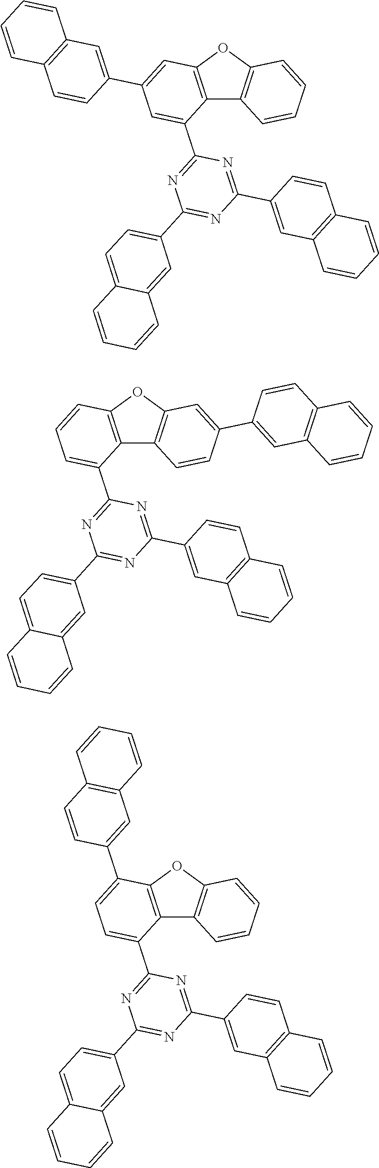

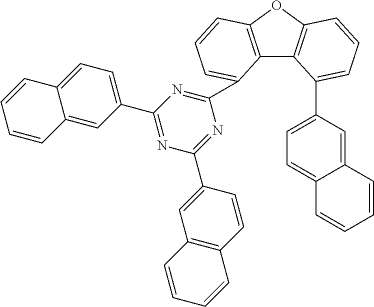 [SMILES: c1ccc2cc(-c3nc(-c4ccc5ccccc5c4)nc(-c4cccc5oc6cccc(-c7ccc8ccccc8c7)c6c45)n3)ccc2c1]

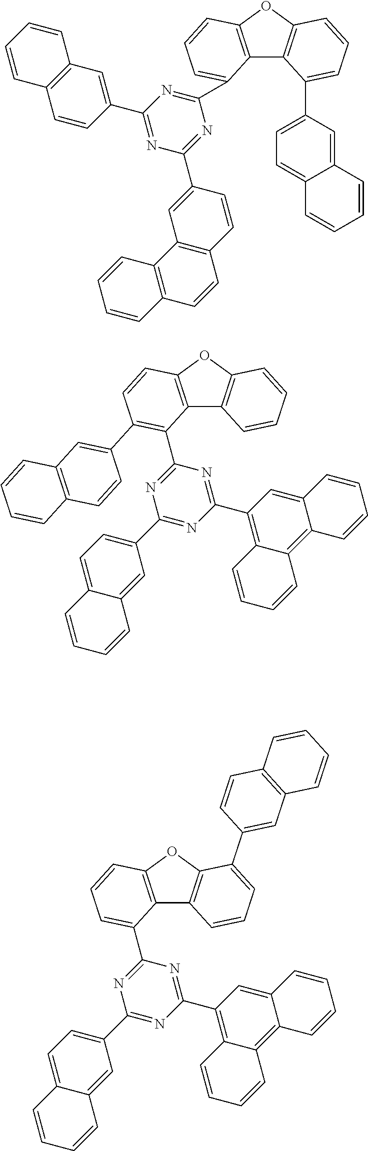 [SMILES: c1ccc2cc(-c3nc(-c4ccc5ccc6ccccc6c5c4)nc(-c4cccc5oc6cccc(-c7ccc8ccccc8c7)c6c45)n3)ccc2c1 ; c1ccc2cc(-c3nc(-c4cc5ccccc5c5ccccc45)nc(-c4c(-c5ccc6ccccc6c5)ccc5oc6ccccc6c45)n3)ccc2c1 ; c1ccc2cc(-c3nc(-c4cc5ccccc5c5ccccc45)nc(-c4cccc5oc6c(-c7ccc8ccccc8c7)cccc6c45)n3)ccc2c1]

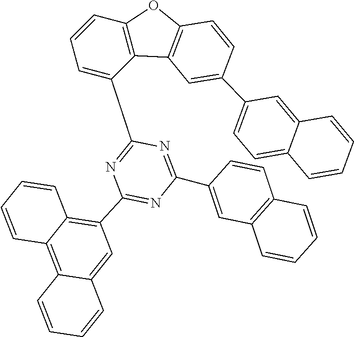 [SMILES: c1ccc2cc(-c3ccc4oc5cccc(-c6nc(-c7ccc8ccccc8c7)nc(-c7cc8ccccc8c8ccccc78)n6)c5c4c3)ccc2c1]

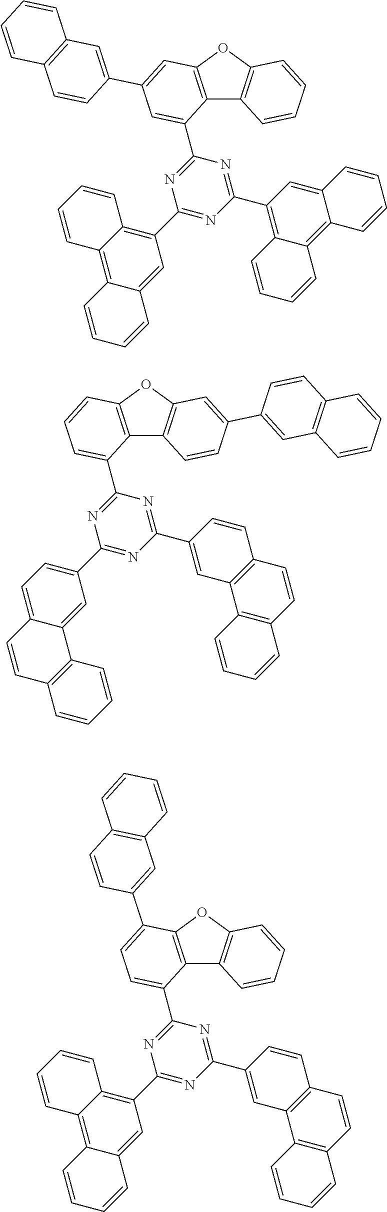

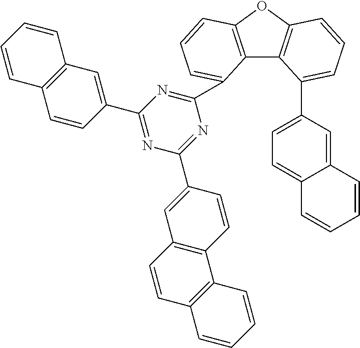 [SMILES: c1ccc2cc(-c3nc(-c4ccc5c(ccc6ccccc65)c4)nc(-c4cccc5oc6cccc(-c7ccc8ccccc8c7)c6c45)n3)ccc2c1]

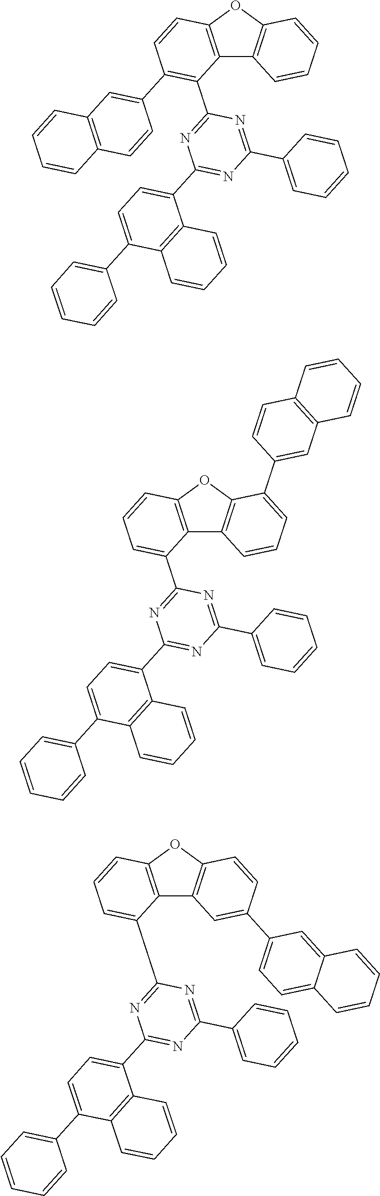 [SMILES: c1ccc(-c2nc(-c3ccc(-c4ccccc4)c4ccccc34)nc(-c3c(-c4ccc5ccccc5c4)ccc4oc5ccccc5c34)n2)cc1 ; c1ccc(-c2nc(-c3ccc(-c4ccccc4)c4ccccc34)nc(-c3cccc4oc5c(-c6ccc7ccccc7c6)cccc5c34)n2)cc1 ; c1ccc(-c2nc(-c3ccc(-c4ccccc4)c4ccccc34)nc(-c3cccc4oc5ccc(-c6ccc7ccccc7c6)cc5c34)n2)cc1]

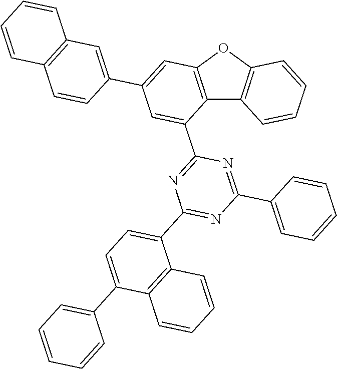 [SMILES: c1ccc(-c2nc(-c3ccc(-c4ccccc4)c4ccccc34)nc(-c3cc(-c4ccc5ccccc5c4)cc4oc5ccccc5c34)n2)cc1]

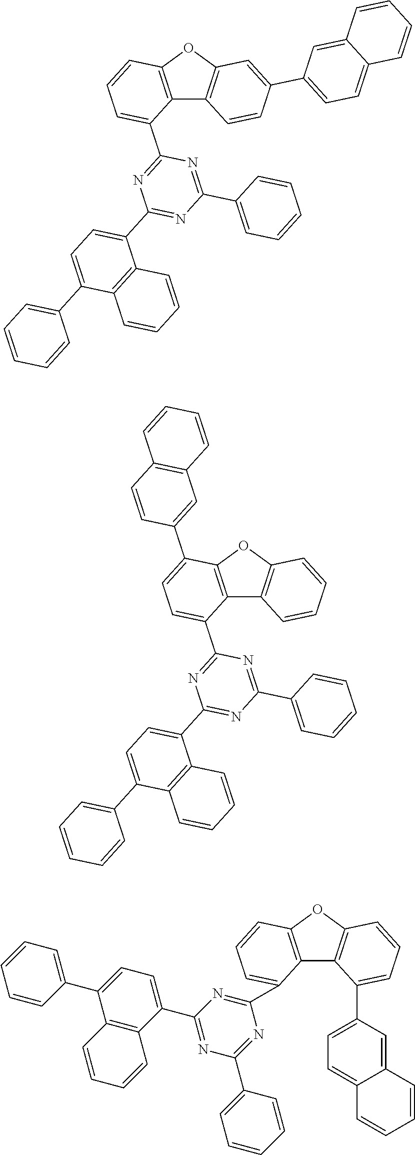 [SMILES: c1ccc(-c2nc(-c3ccc(-c4ccccc4)c4ccccc34)nc(-c3cccc4oc5cc(-c6ccc7ccccc7c6)ccc5c34)n2)cc1 ; c1ccc(-c2nc(-c3ccc(-c4ccccc4)c4ccccc34)nc(-c3ccc(-c4ccc5ccccc5c4)c4oc5ccccc5c34)n2)cc1 ; c1ccc(-c2nc(-c3ccc(-c4ccccc4)c4ccccc34)nc(-c3cccc4oc5cccc(-c6ccc7ccccc7c6)c5c34)n2)cc1]

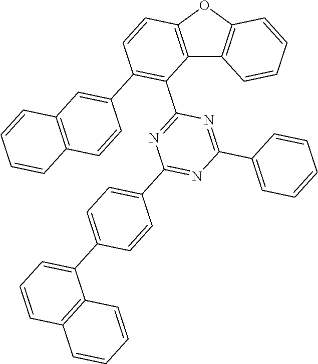 [SMILES: c1ccc(-c2nc(-c3ccc(-c4cccc5ccccc45)cc3)nc(-c3c(-c4ccc5ccccc5c4)ccc4oc5ccccc5c34)n2)cc1]

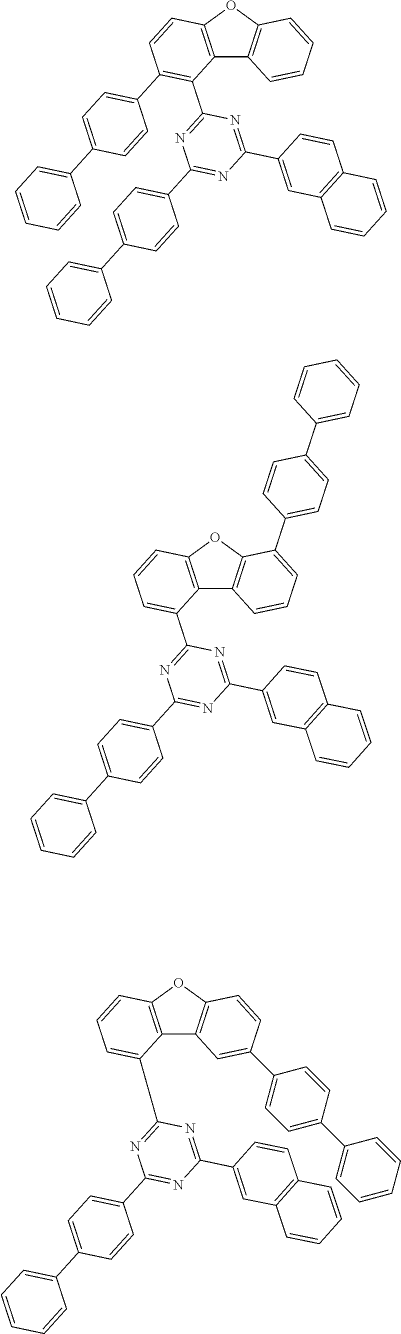 [SMILES: c1ccc(-c2ccc(-c3nc(-c4ccc5ccccc5c4)nc(-c4c(-c5ccc(-c6ccccc6)cc5)ccc5oc6ccccc6c45)n3)cc2)cc1 ; c1ccc(-c2ccc(-c3nc(-c4ccc5ccccc5c4)nc(-c4cccc5oc6c(-c7ccc(-c8ccccc8)cc7)cccc6c45)n3)cc2)cc1 ; c1ccc(-c2ccc(-c3ccc4oc5cccc(-c6nc(-c7ccc(-c8ccccc8)cc7)nc(-c7ccc8ccccc8c7)n6)c5c4c3)cc2)cc1]

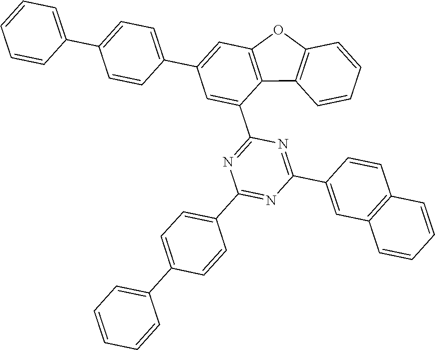 [SMILES: c1ccc(-c2ccc(-c3cc(-c4nc(-c5ccc(-c6ccccc6)cc5)nc(-c5ccc6ccccc6c5)n4)c4c(c3)oc3ccccc34)cc2)cc1]

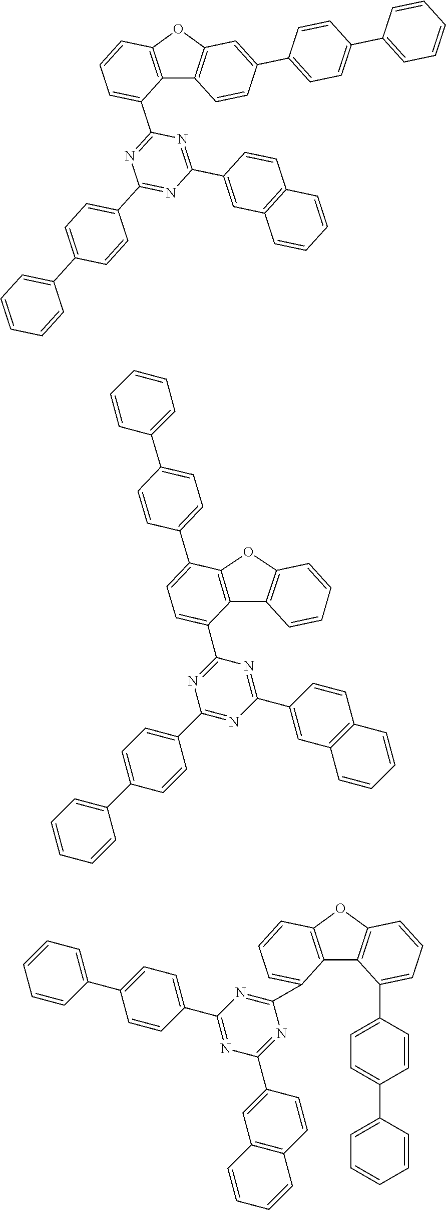 [SMILES: c1ccc(-c2ccc(-c3ccc4c(c3)oc3cccc(-c5nc(-c6ccc(-c7ccccc7)cc6)nc(-c6ccc7ccccc7c6)n5)c34)cc2)cc1 ; c1ccc(-c2ccc(-c3nc(-c4ccc5ccccc5c4)nc(-c4ccc(-c5ccc(-c6ccccc6)cc5)c5oc6ccccc6c45)n3)cc2)cc1 ; c1ccc(-c2ccc(-c3nc(-c4ccc5ccccc5c4)nc(-c4cccc5oc6cccc(-c7ccc(-c8ccccc8)cc7)c6c45)n3)cc2)cc1]

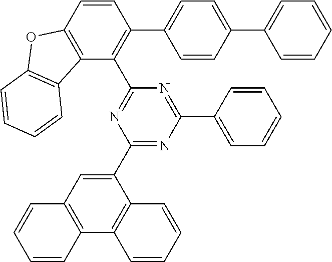 [SMILES: c1ccc(-c2ccc(-c3ccc4oc5ccccc5c4c3-c3nc(-c4ccccc4)nc(-c4cc5ccccc5c5ccccc45)n3)cc2)cc1]

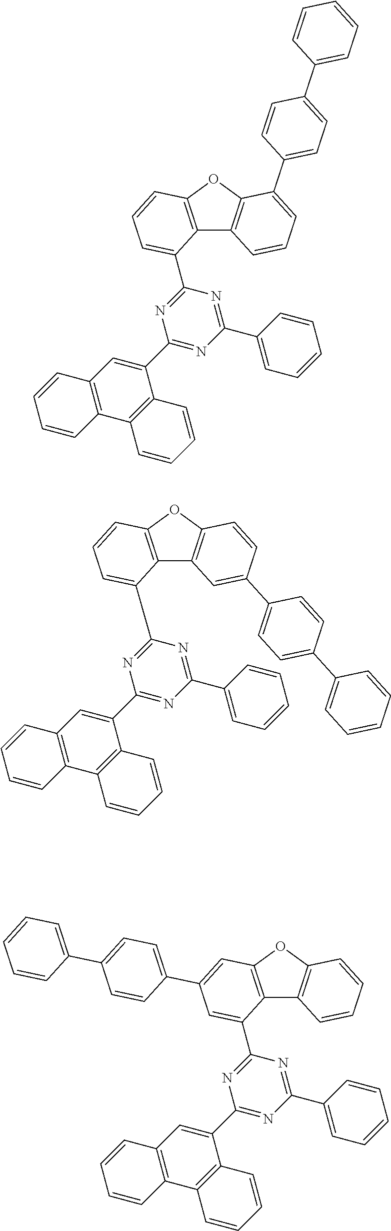

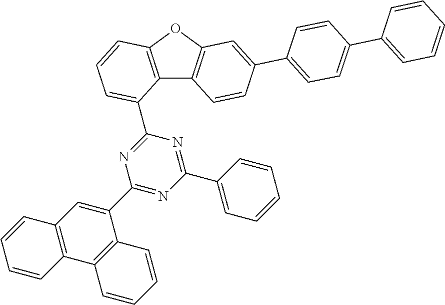 [SMILES: c1ccc(-c2ccc(-c3ccc4c(c3)oc3cccc(-c5nc(-c6ccccc6)nc(-c6cc7ccccc7c7ccccc67)n5)c34)cc2)cc1]

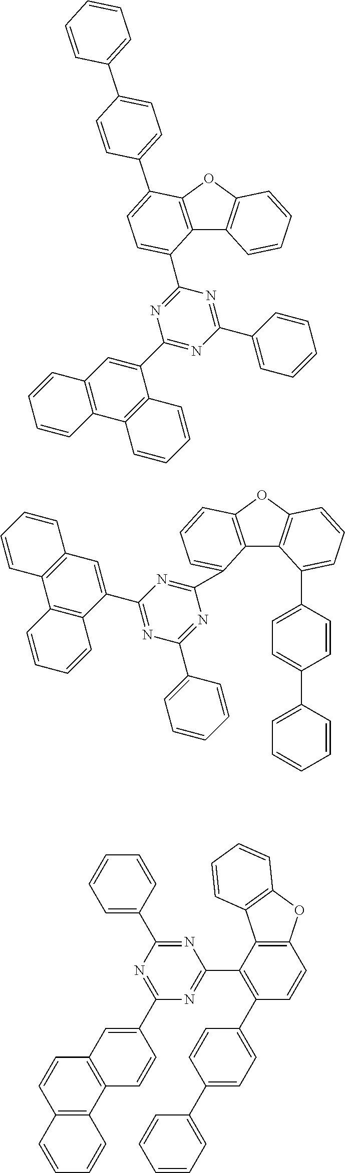 [SMILES: c1ccc(-c2ccc(-c3ccc(-c4nc(-c5ccccc5)nc(-c5cc6ccccc6c6ccccc56)n4)c4c3oc3ccccc34)cc2)cc1 ; c1ccc(-c2ccc(-c3cccc4oc5cccc(-c6nc(-c7ccccc7)nc(-c7cc8ccccc8c8ccccc78)n6)c5c34)cc2)cc1 ; c1ccc(-c2ccc(-c3ccc4oc5ccccc5c4c3-c3nc(-c4ccccc4)nc(-c4ccc5c(ccc6ccccc65)c4)n3)cc2)cc1]

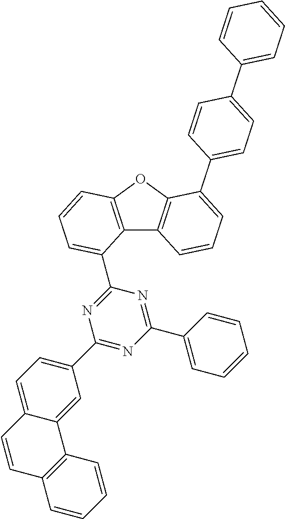 [SMILES: c1ccc(-c2ccc(-c3cccc4c3oc3cccc(-c5nc(-c6ccccc6)nc(-c6ccc7ccc8ccccc8c7c6)n5)c34)cc2)cc1]

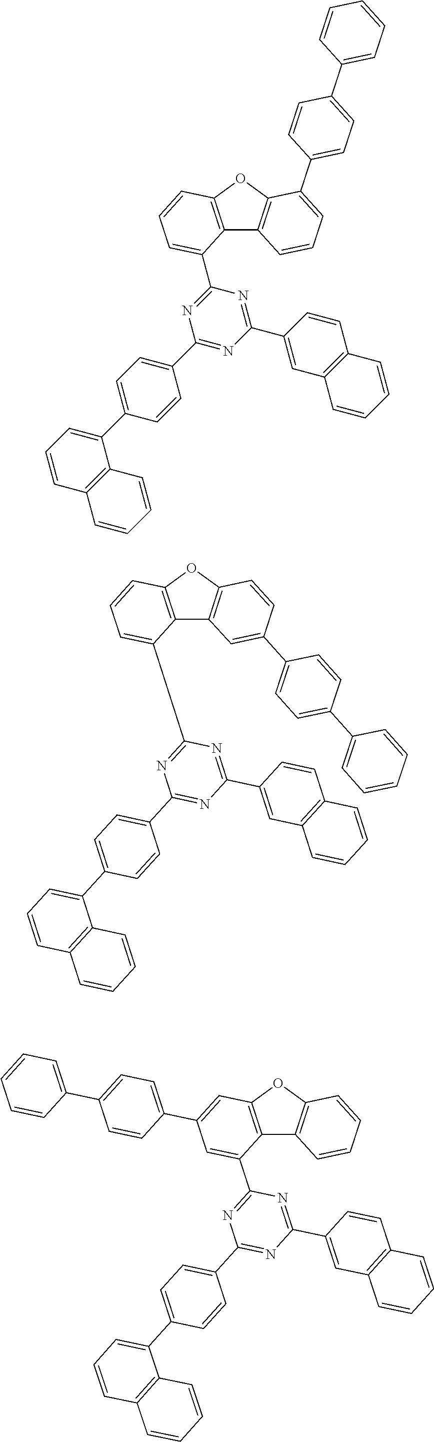 [SMILES: c1ccc(-c2ccc(-c3cccc4c3oc3cccc(-c5nc(-c6ccc(-c7cccc8ccccc78)cc6)nc(-c6ccc7ccccc7c6)n5)c34)cc2)cc1 ; c1ccc(-c2ccc(-c3ccc4oc5cccc(-c6nc(-c7ccc(-c8cccc9ccccc89)cc7)nc(-c7ccc8ccccc8c7)n6)c5c4c3)cc2)cc1 ; c1ccc(-c2ccc(-c3cc(-c4nc(-c5ccc(-c6cccc7ccccc67)cc5)nc(-c5ccc6ccccc6c5)n4)c4c(c3)oc3ccccc34)cc2)cc1]

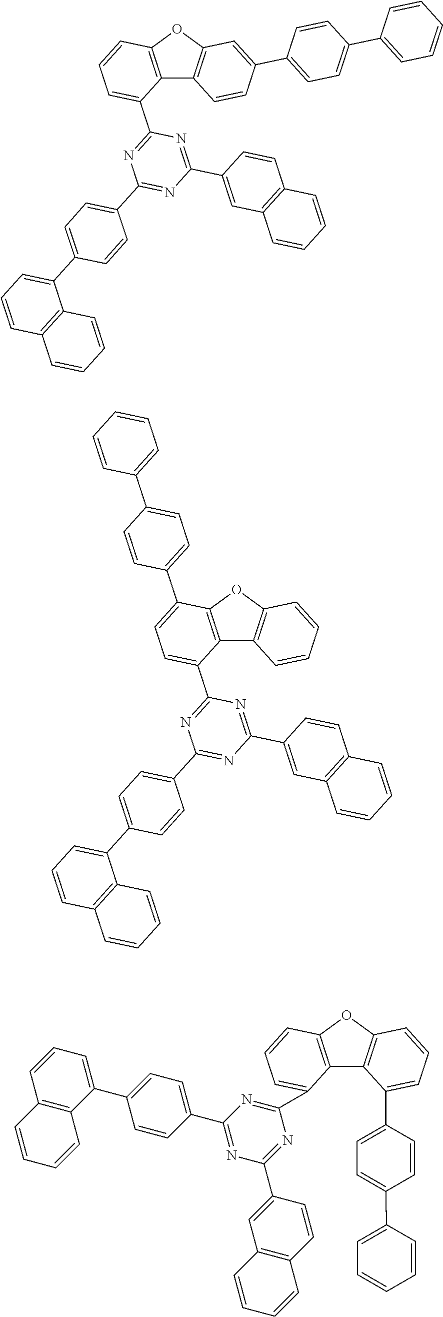 [SMILES: c1ccc(-c2ccc(-c3ccc4c(c3)oc3cccc(-c5nc(-c6ccc(-c7cccc8ccccc78)cc6)nc(-c6ccc7ccccc7c6)n5)c34)cc2)cc1 ; c1ccc(-c2ccc(-c3ccc(-c4nc(-c5ccc(-c6cccc7ccccc67)cc5)nc(-c5ccc6ccccc6c5)n4)c4c3oc3ccccc34)cc2)cc1 ; c1ccc(-c2ccc(-c3cccc4oc5cccc(-c6nc(-c7ccc(-c8cccc9ccccc89)cc7)nc(-c7ccc8ccccc8c7)n6)c5c34)cc2)cc1]

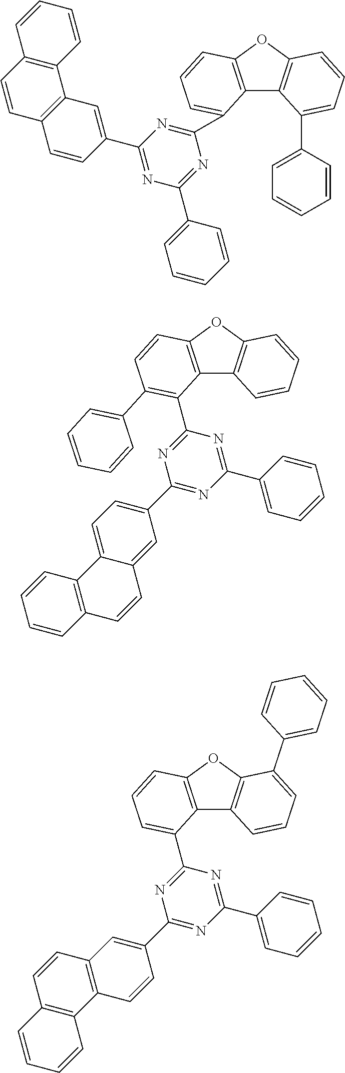

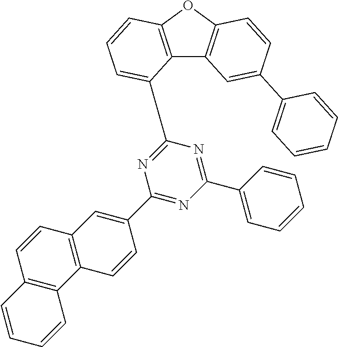 [SMILES: c1ccc(-c2ccc3oc4cccc(-c5nc(-c6ccccc6)nc(-c6ccc7c(ccc8ccccc87)c6)n5)c4c3c2)cc1]

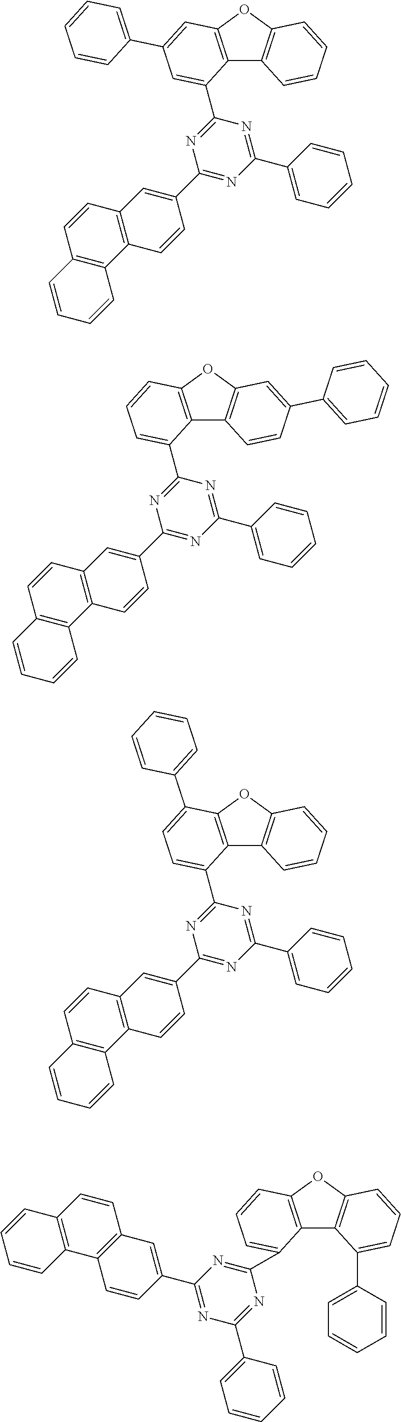 [SMILES: c1ccc(-c2cc(-c3nc(-c4ccccc4)nc(-c4ccc5c(ccc6ccccc65)c4)n3)c3c(c2)oc2ccccc23)cc1 ; c1ccc(-c2ccc3c(c2)oc2cccc(-c4nc(-c5ccccc5)nc(-c5ccc6c(ccc7ccccc76)c5)n4)c23)cc1 ; c1ccc(-c2nc(-c3ccc4c(ccc5ccccc54)c3)nc(-c3ccc(-c4ccccc4)c4oc5ccccc5c34)n2)cc1 ; c1ccc(-c2nc(-c3ccc4c(ccc5ccccc54)c3)nc(-c3cccc4oc5cccc(-c6ccccc6)c5c34)n2)cc1]

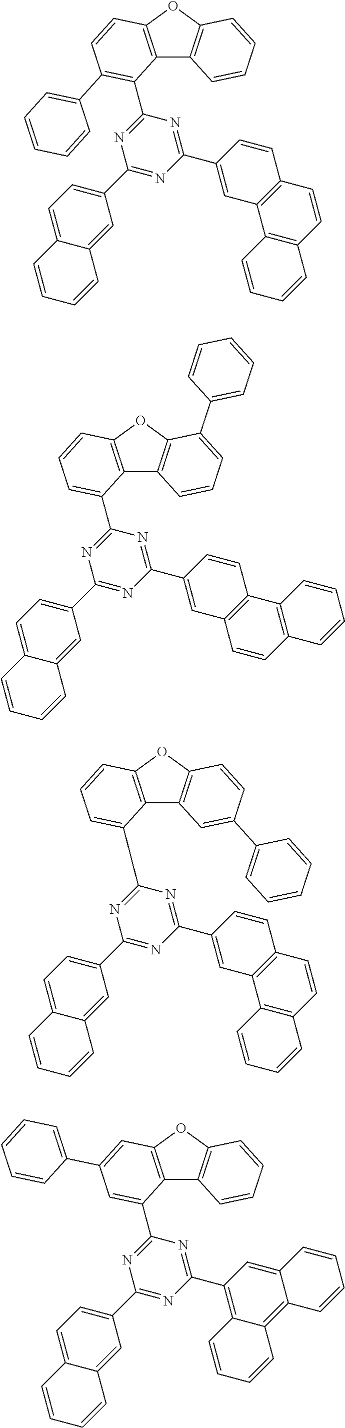 [SMILES: c1ccc(-c2ccc3oc4ccccc4c3c2-c2nc(-c3ccc4ccccc4c3)nc(-c3ccc4ccc5ccccc5c4c3)n2)cc1 ; c1ccc(-c2cccc3c2oc2cccc(-c4nc(-c5ccc6ccccc6c5)nc(-c5ccc6c(ccc7ccccc76)c5)n4)c23)cc1 ; c1ccc(-c2ccc3oc4cccc(-c5nc(-c6ccc7ccccc7c6)nc(-c6ccc7ccc8ccccc8c7c6)n5)c4c3c2)cc1 ; c1ccc(-c2cc(-c3nc(-c4ccc5ccccc5c4)nc(-c4cc5ccccc5c5ccccc45)n3)c3c(c2)oc2ccccc23)cc1]

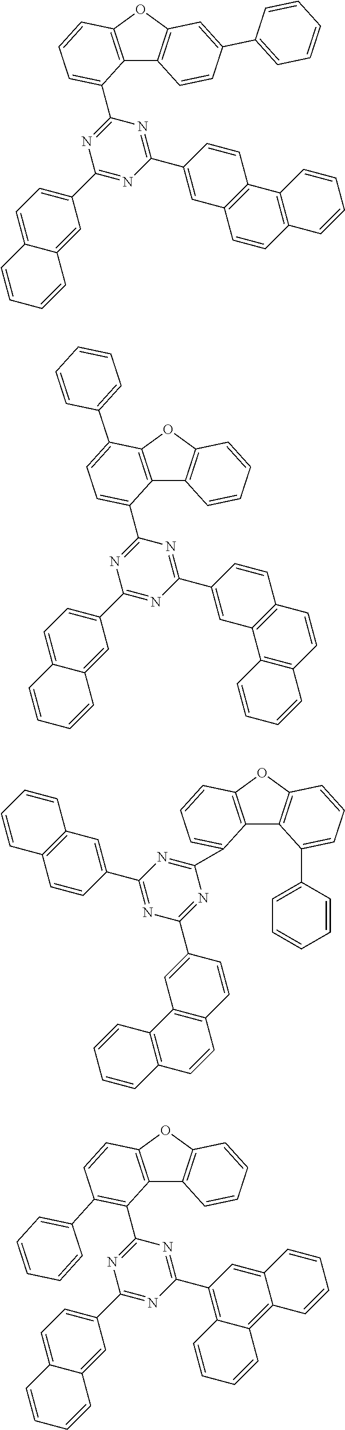 [SMILES: c1ccc(-c2ccc3c(c2)oc2cccc(-c4nc(-c5ccc6ccccc6c5)nc(-c5ccc6c(ccc7ccccc76)c5)n4)c23)cc1 ; c1ccc(-c2ccc(-c3nc(-c4ccc5ccccc5c4)nc(-c4ccc5ccc6ccccc6c5c4)n3)c3c2oc2ccccc23)cc1 ; c1ccc(-c2cccc3oc4cccc(-c5nc(-c6ccc7ccccc7c6)nc(-c6ccc7ccc8ccccc8c7c6)n5)c4c23)cc1 ; c1ccc(-c2ccc3oc4ccccc4c3c2-c2nc(-c3ccc4ccccc4c3)nc(-c3cc4ccccc4c4ccccc34)n2)cc1]

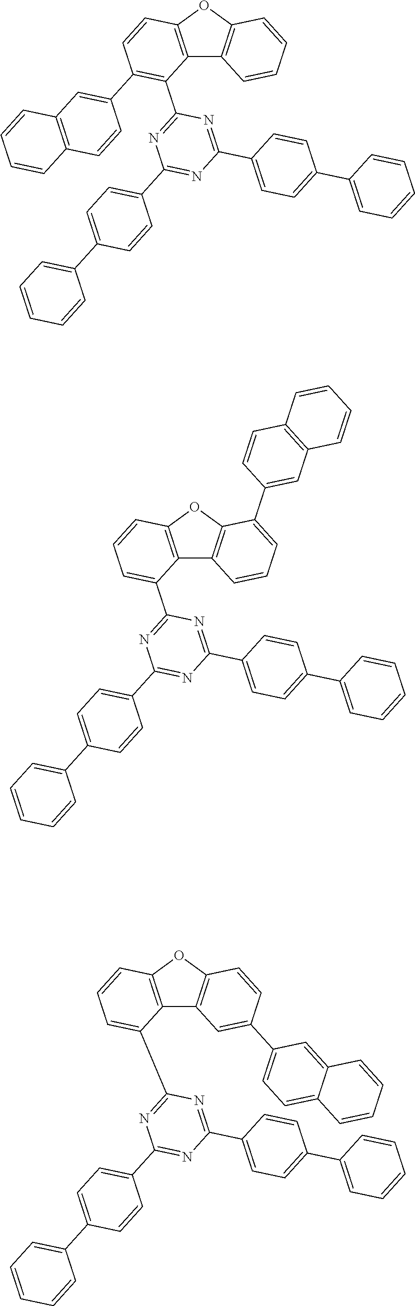 [SMILES: c1ccc(-c2ccc(-c3nc(-c4ccc(-c5ccccc5)cc4)nc(-c4c(-c5ccc6ccccc6c5)ccc5oc6ccccc6c45)n3)cc2)cc1 ; c1ccc(-c2ccc(-c3nc(-c4ccc(-c5ccccc5)cc4)nc(-c4cccc5oc6c(-c7ccc8ccccc8c7)cccc6c45)n3)cc2)cc1 ; c1ccc(-c2ccc(-c3nc(-c4ccc(-c5ccccc5)cc4)nc(-c4cccc5oc6ccc(-c7ccc8ccccc8c7)cc6c45)n3)cc2)cc1]

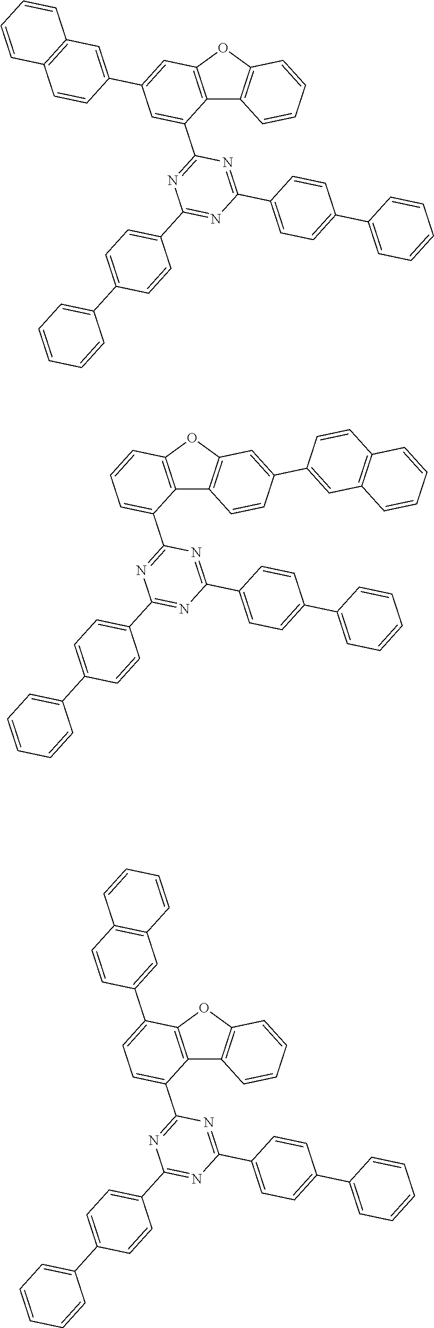 [SMILES: c1ccc(-c2ccc(-c3nc(-c4ccc(-c5ccccc5)cc4)nc(-c4cc(-c5ccc6ccccc6c5)cc5oc6ccccc6c45)n3)cc2)cc1 ; c1ccc(-c2ccc(-c3nc(-c4ccc(-c5ccccc5)cc4)nc(-c4cccc5oc6cc(-c7ccc8ccccc8c7)ccc6c45)n3)cc2)cc1 ; c1ccc(-c2ccc(-c3nc(-c4ccc(-c5ccccc5)cc4)nc(-c4ccc(-c5ccc6ccccc6c5)c5oc6ccccc6c45)n3)cc2)cc1]

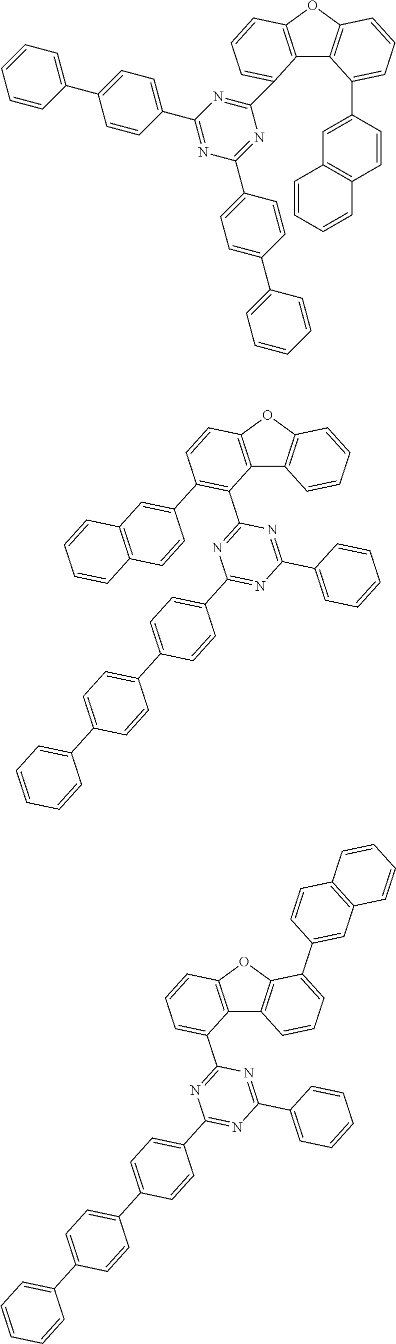 [SMILES: c1ccc(-c2ccc(-c3nc(-c4ccc(-c5ccccc5)cc4)nc(-c4cccc5oc6cccc(-c7ccc8ccccc8c7)c6c45)n3)cc2)cc1 ; c1ccc(-c2ccc(-c3ccc(-c4nc(-c5ccccc5)nc(-c5c(-c6ccc7ccccc7c6)ccc6oc7ccccc7c56)n4)cc3)cc2)cc1 ; c1ccc(-c2ccc(-c3ccc(-c4nc(-c5ccccc5)nc(-c5cccc6oc7c(-c8ccc9ccccc9c8)cccc7c56)n4)cc3)cc2)cc1]

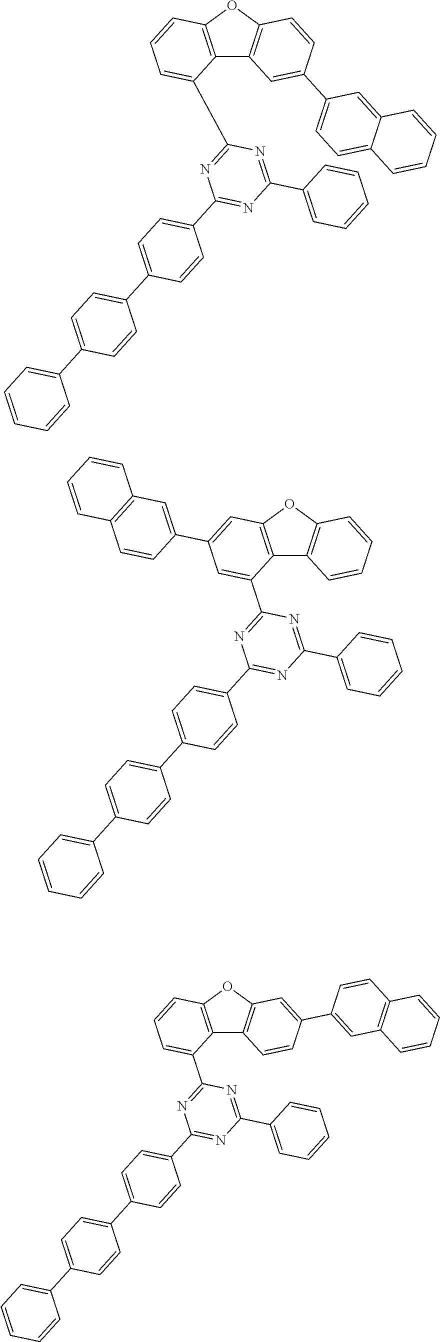 [SMILES: c1ccc(-c2ccc(-c3ccc(-c4nc(-c5ccccc5)nc(-c5cccc6oc7ccc(-c8ccc9ccccc9c8)cc7c56)n4)cc3)cc2)cc1 ; c1ccc(-c2ccc(-c3ccc(-c4nc(-c5ccccc5)nc(-c5cc(-c6ccc7ccccc7c6)cc6oc7ccccc7c56)n4)cc3)cc2)cc1 ; c1ccc(-c2ccc(-c3ccc(-c4nc(-c5ccccc5)nc(-c5cccc6oc7cc(-c8ccc9ccccc9c8)ccc7c56)n4)cc3)cc2)cc1]

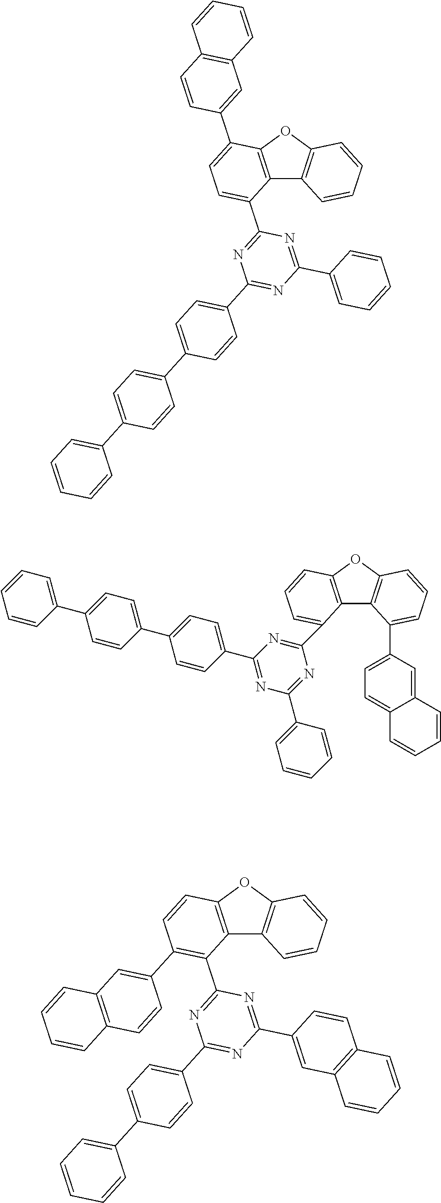 [SMILES: c1ccc(-c2ccc(-c3ccc(-c4nc(-c5ccccc5)nc(-c5ccc(-c6ccc7ccccc7c6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9ccccc9c8)c7c56)n4)cc3)cc2)cc1 ; c1ccc(-c2ccc(-c3nc(-c4ccc5ccccc5c4)nc(-c4c(-c5ccc6ccccc6c5)ccc5oc6ccccc6c45)n3)cc2)cc1]

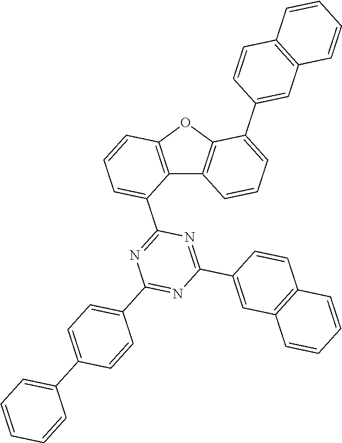 [SMILES: c1ccc(-c2ccc(-c3nc(-c4ccc5ccccc5c4)nc(-c4cccc5oc6c(-c7ccc8ccccc8c7)cccc6c45)n3)cc2)cc1]

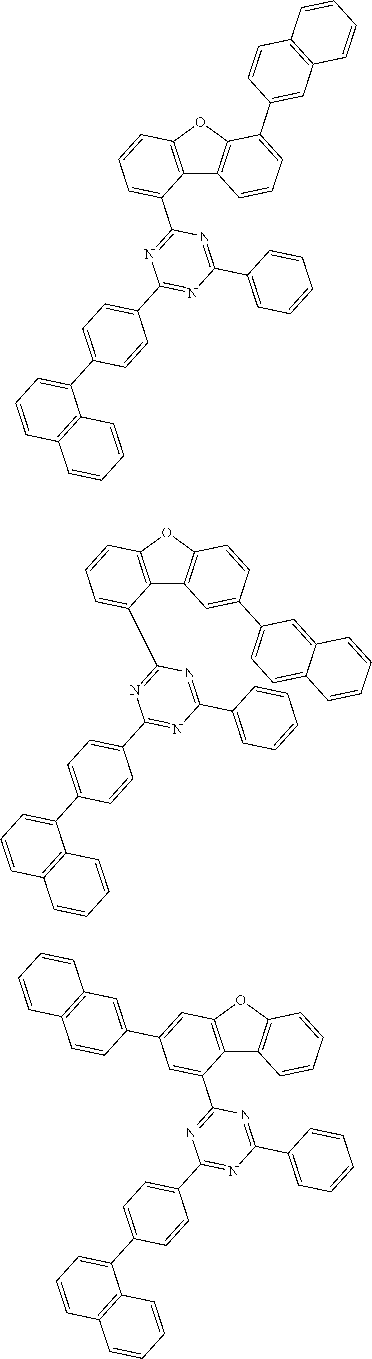

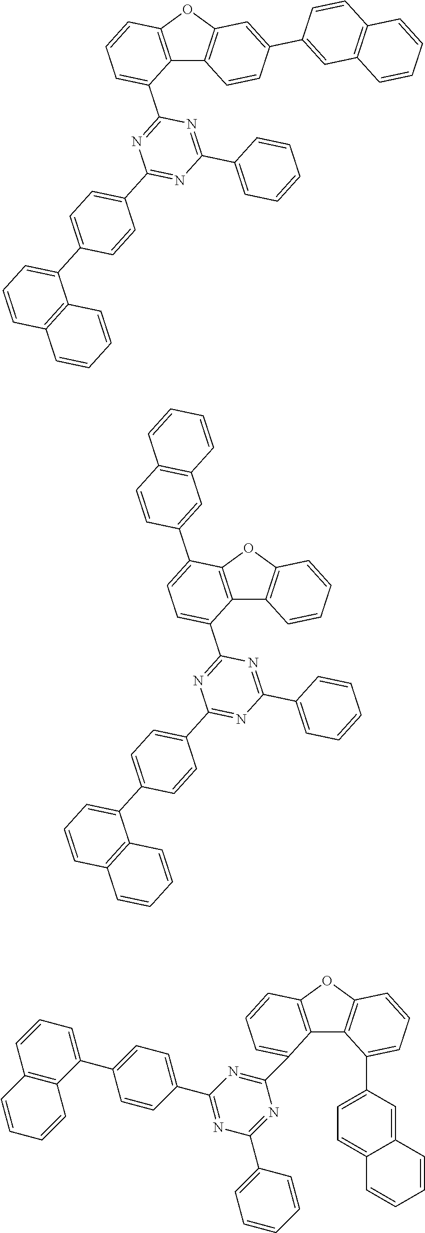 [SMILES: c1ccc(-c2nc(-c3ccc(-c4cccc5ccccc45)cc3)nc(-c3cccc4oc5cc(-c6ccc7ccccc7c6)ccc5c34)n2)cc1 ; c1ccc(-c2nc(-c3ccc(-c4cccc5ccccc45)cc3)nc(-c3ccc(-c4ccc5ccccc5c4)c4oc5ccccc5c34)n2)cc1 ; c1ccc(-c2nc(-c3ccc(-c4cccc5ccccc45)cc3)nc(-c3cccc4oc5cccc(-c6ccc7ccccc7c6)c5c34)n2)cc1]

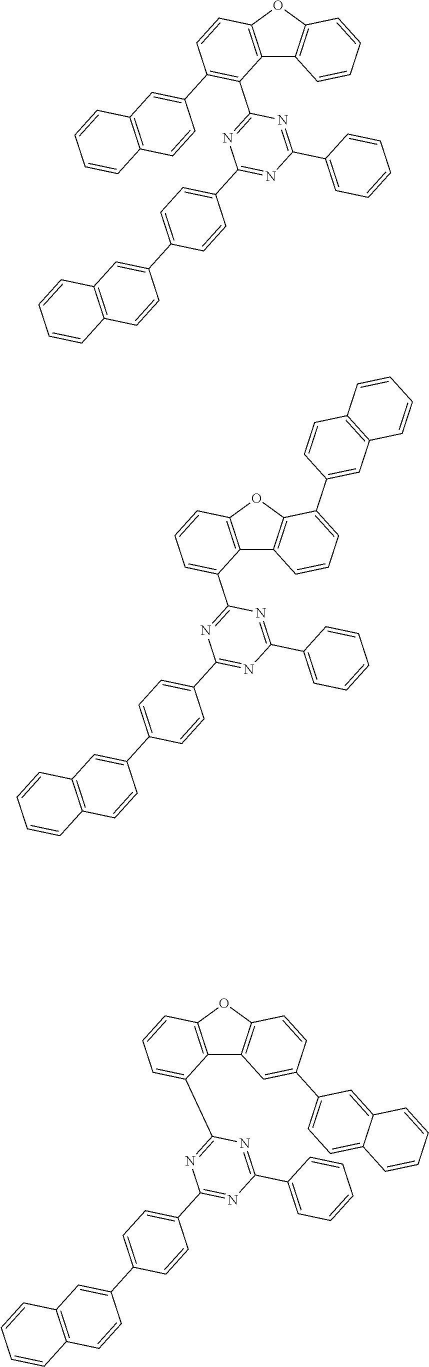 [SMILES: c1ccc(-c2nc(-c3ccc(-c4ccc5ccccc5c4)cc3)nc(-c3c(-c4ccc5ccccc5c4)ccc4oc5ccccc5c34)n2)cc1 ; c1ccc(-c2nc(-c3ccc(-c4ccc5ccccc5c4)cc3)nc(-c3cccc4oc5c(-c6ccc7ccccc7c6)cccc5c34)n2)cc1 ; c1ccc(-c2nc(-c3ccc(-c4ccc5ccccc5c4)cc3)nc(-c3cccc4oc5ccc(-c6ccc7ccccc7c6)cc5c34)n2)cc1]

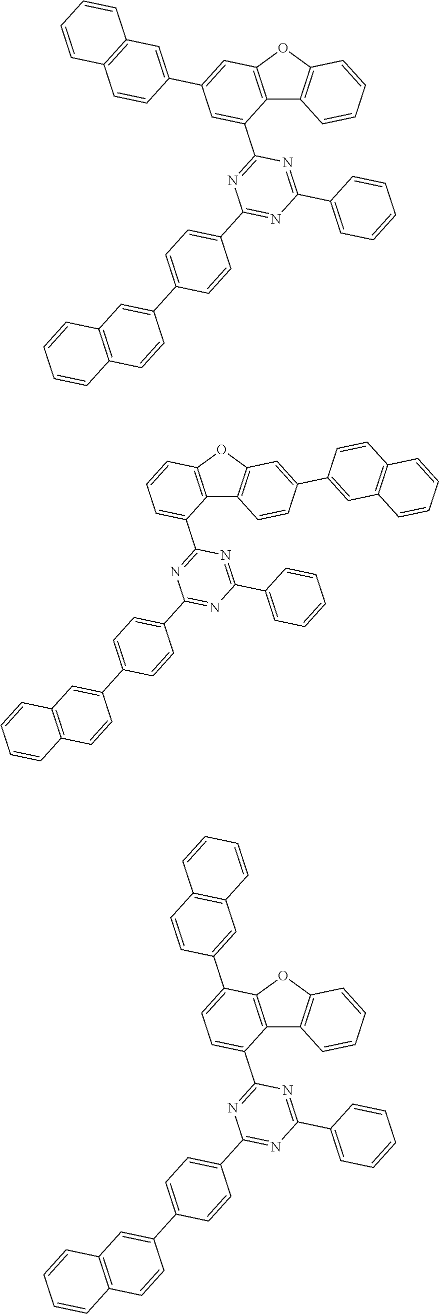 [SMILES: c1ccc(-c2nc(-c3ccc(-c4ccc5ccccc5c4)cc3)nc(-c3cc(-c4ccc5ccccc5c4)cc4oc5ccccc5c34)n2)cc1 ; c1ccc(-c2nc(-c3ccc(-c4ccc5ccccc5c4)cc3)nc(-c3cccc4oc5cc(-c6ccc7ccccc7c6)ccc5c34)n2)cc1 ; c1ccc(-c2nc(-c3ccc(-c4ccc5ccccc5c4)cc3)nc(-c3ccc(-c4ccc5ccccc5c4)c4oc5ccccc5c34)n2)cc1]

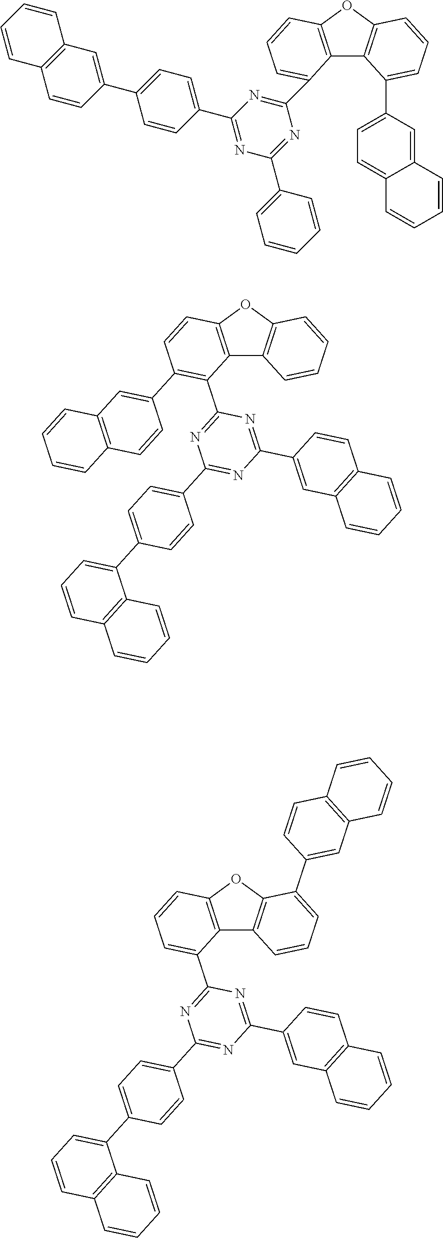 [SMILES: c1ccc(-c2nc(-c3ccc(-c4ccc5ccccc5c4)cc3)nc(-c3cccc4oc5cccc(-c6ccc7ccccc7c6)c5c34)n2)cc1 ; c1ccc2cc(-c3nc(-c4ccc(-c5cccc6ccccc56)cc4)nc(-c4c(-c5ccc6ccccc6c5)ccc5oc6ccccc6c45)n3)ccc2c1 ; c1ccc2cc(-c3nc(-c4ccc(-c5cccc6ccccc56)cc4)nc(-c4cccc5oc6c(-c7ccc8ccccc8c7)cccc6c45)n3)ccc2c1]

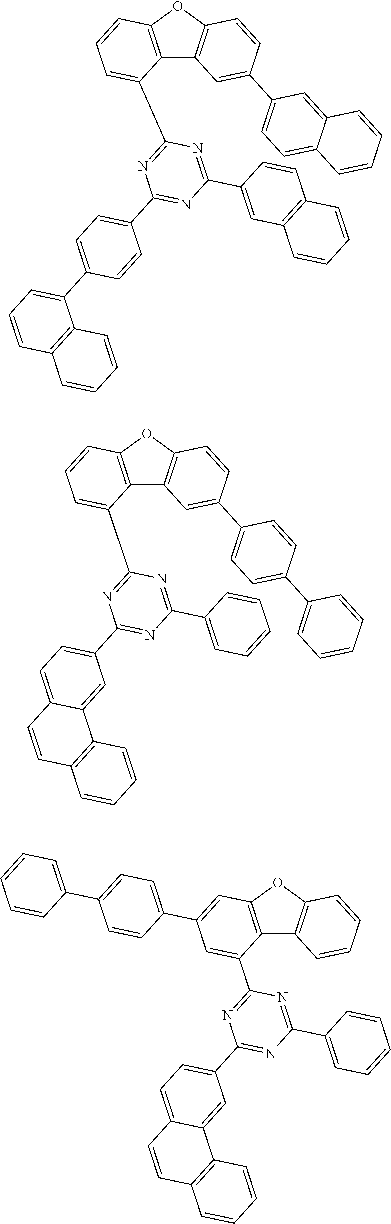 [SMILES: c1ccc2cc(-c3ccc4oc5cccc(-c6nc(-c7ccc(-c8cccc9ccccc89)cc7)nc(-c7ccc8ccccc8c7)n6)c5c4c3)ccc2c1 ; c1ccc(-c2ccc(-c3ccc4oc5cccc(-c6nc(-c7ccccc7)nc(-c7ccc8ccc9ccccc9c8c7)n6)c5c4c3)cc2)cc1 ; c1ccc(-c2ccc(-c3cc(-c4nc(-c5ccccc5)nc(-c5ccc6ccc7ccccc7c6c5)n4)c4c(c3)oc3ccccc34)cc2)cc1]

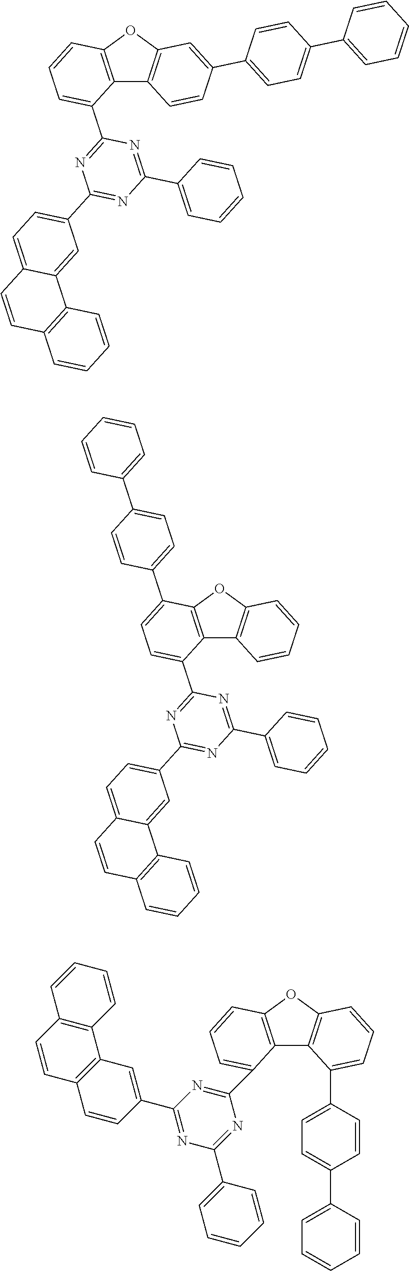 [SMILES: c1ccc(-c2ccc(-c3ccc4c(c3)oc3cccc(-c5nc(-c6ccccc6)nc(-c6ccc7ccc8ccccc8c7c6)n5)c34)cc2)cc1 ; c1ccc(-c2ccc(-c3ccc(-c4nc(-c5ccccc5)nc(-c5ccc6ccc7ccccc7c6c5)n4)c4c3oc3ccccc34)cc2)cc1 ; c1ccc(-c2ccc(-c3cccc4oc5cccc(-c6nc(-c7ccccc7)nc(-c7ccc8ccc9ccccc9c8c7)n6)c5c34)cc2)cc1]

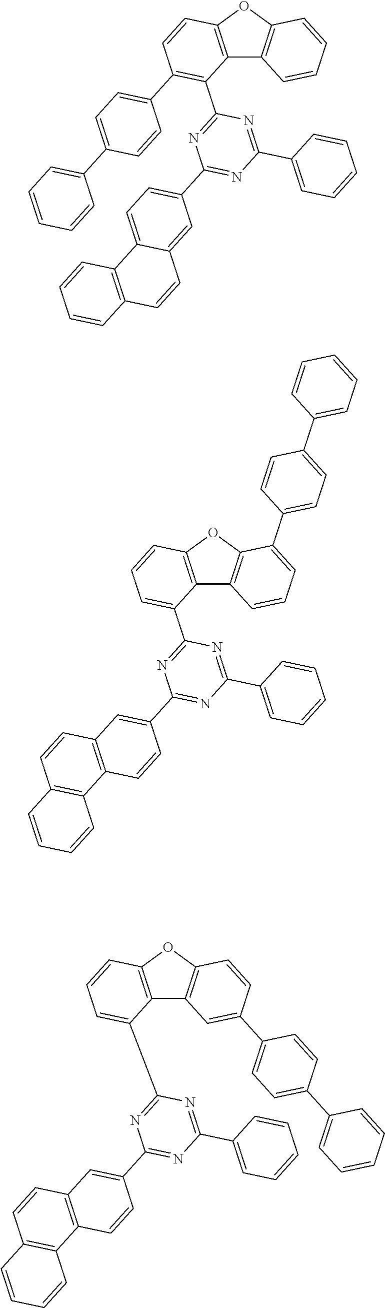 [SMILES: c1ccc(-c2ccc(-c3ccc4oc5ccccc5c4c3-c3nc(-c4ccccc4)nc(-c4ccc5c(ccc6ccccc65)c4)n3)cc2)cc1 ; c1ccc(-c2ccc(-c3cccc4c3oc3cccc(-c5nc(-c6ccccc6)nc(-c6ccc7c(ccc8ccccc87)c6)n5)c34)cc2)cc1 ; c1ccc(-c2ccc(-c3ccc4oc5cccc(-c6nc(-c7ccccc7)nc(-c7ccc8c(ccc9ccccc98)c7)n6)c5c4c3)cc2)cc1]

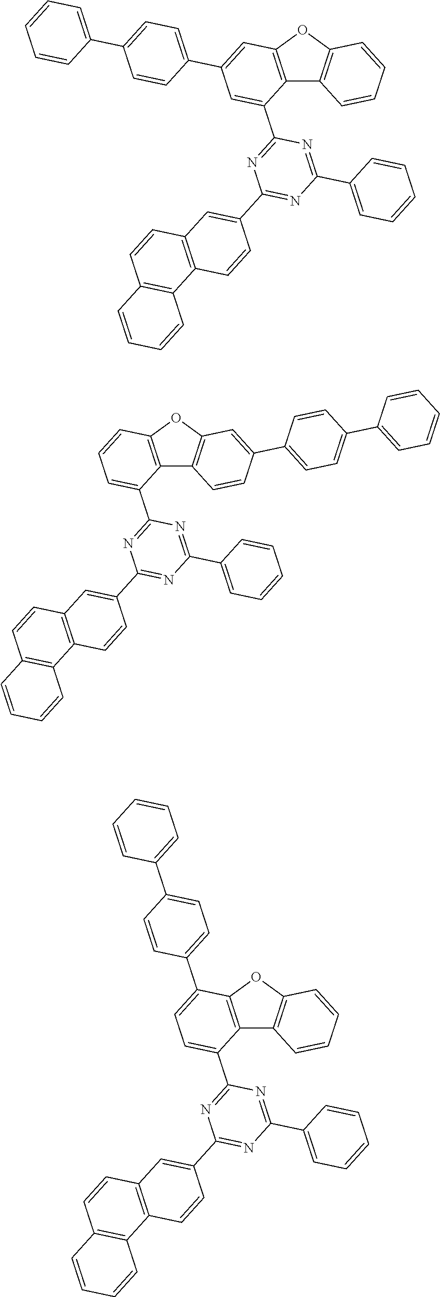 [SMILES: c1ccc(-c2ccc(-c3cc(-c4nc(-c5ccccc5)nc(-c5ccc6c(ccc7ccccc76)c5)n4)c4c(c3)oc3ccccc34)cc2)cc1 ; c1ccc(-c2ccc(-c3ccc4c(c3)oc3cccc(-c5nc(-c6ccccc6)nc(-c6ccc7c(ccc8ccccc87)c6)n5)c34)cc2)cc1 ; c1ccc(-c2ccc(-c3ccc(-c4nc(-c5ccccc5)nc(-c5ccc6c(ccc7ccccc76)c5)n4)c4c3oc3ccccc34)cc2)cc1]

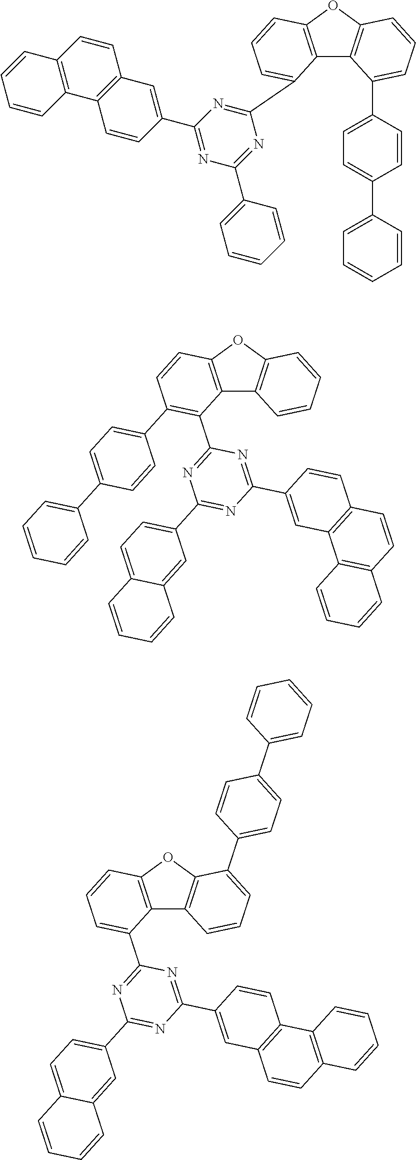 [SMILES: c1ccc(-c2ccc(-c3cccc4oc5cccc(-c6nc(-c7ccccc7)nc(-c7ccc8c(ccc9ccccc98)c7)n6)c5c34)cc2)cc1 ; c1ccc(-c2ccc(-c3ccc4oc5ccccc5c4c3-c3nc(-c4ccc5ccccc5c4)nc(-c4ccc5ccc6ccccc6c5c4)n3)cc2)cc1 ; c1ccc(-c2ccc(-c3cccc4c3oc3cccc(-c5nc(-c6ccc7ccccc7c6)nc(-c6ccc7c(ccc8ccccc87)c6)n5)c34)cc2)cc1]

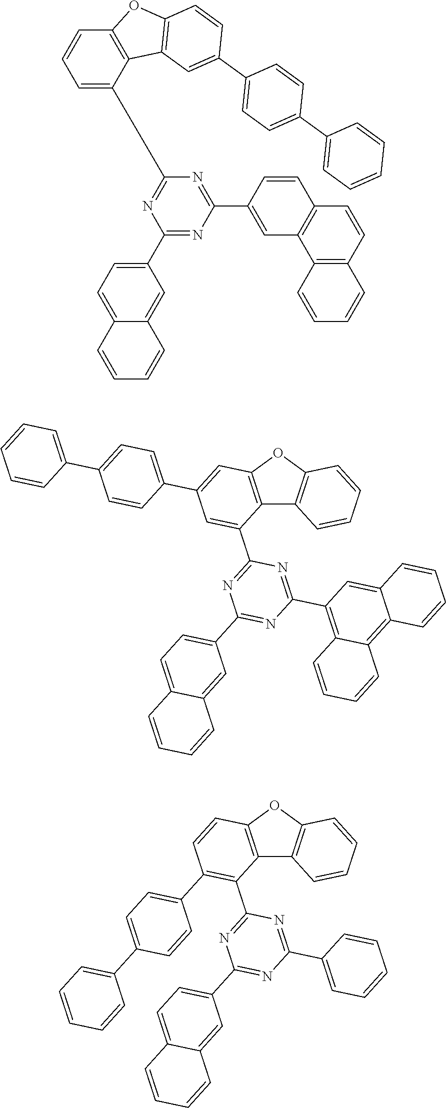 [SMILES: c1ccc(-c2ccc(-c3ccc4oc5cccc(-c6nc(-c7ccc8ccccc8c7)nc(-c7ccc8ccc9ccccc9c8c7)n6)c5c4c3)cc2)cc1 ; c1ccc(-c2ccc(-c3cc(-c4nc(-c5ccc6ccccc6c5)nc(-c5cc6ccccc6c6ccccc56)n4)c4c(c3)oc3ccccc34)cc2)cc1 ; c1ccc(-c2ccc(-c3ccc4oc5ccccc5c4c3-c3nc(-c4ccccc4)nc(-c4ccc5ccccc5c4)n3)cc2)cc1]

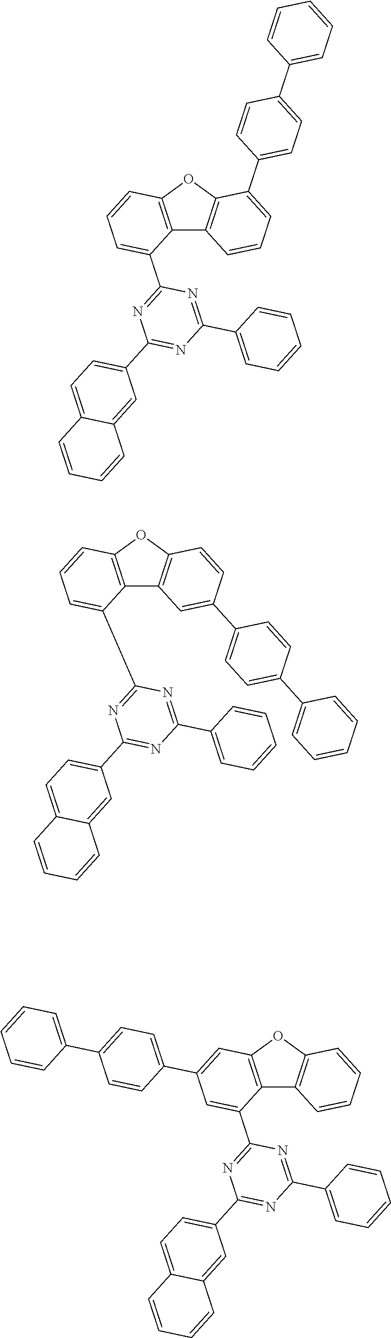 [SMILES: c1ccc(-c2ccc(-c3cccc4c3oc3cccc(-c5nc(-c6ccccc6)nc(-c6ccc7ccccc7c6)n5)c34)cc2)cc1 ; c1ccc(-c2ccc(-c3ccc4oc5cccc(-c6nc(-c7ccccc7)nc(-c7ccc8ccccc8c7)n6)c5c4c3)cc2)cc1 ; c1ccc(-c2ccc(-c3cc(-c4nc(-c5ccccc5)nc(-c5ccc6ccccc6c5)n4)c4c(c3)oc3ccccc34)cc2)cc1]

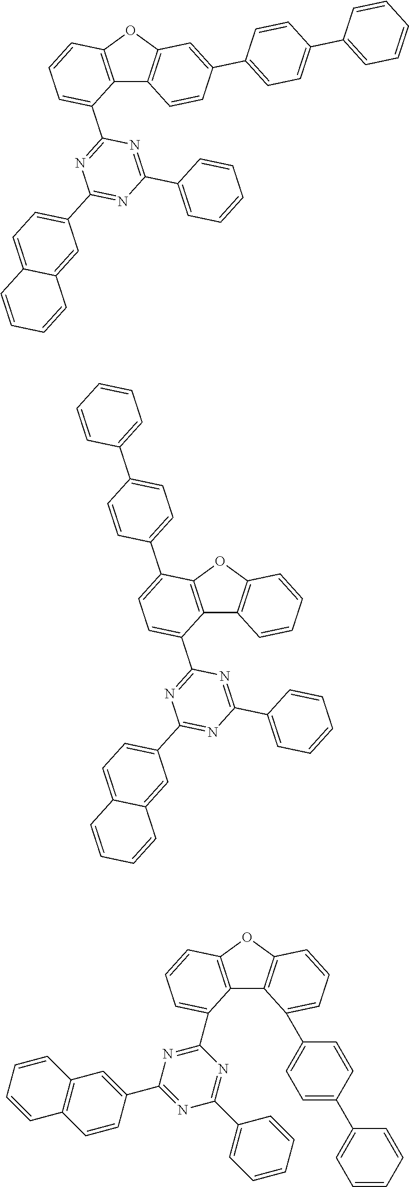 [SMILES: c1ccc(-c2ccc(-c3ccc4c(c3)oc3cccc(-c5nc(-c6ccccc6)nc(-c6ccc7ccccc7c6)n5)c34)cc2)cc1 ; c1ccc(-c2ccc(-c3ccc(-c4nc(-c5ccccc5)nc(-c5ccc6ccccc6c5)n4)c4c3oc3ccccc34)cc2)cc1 ; c1ccc(-c2ccc(-c3cccc4oc5cccc(-c6nc(-c7ccccc7)nc(-c7ccc8ccccc8c7)n6)c5c34)cc2)cc1]

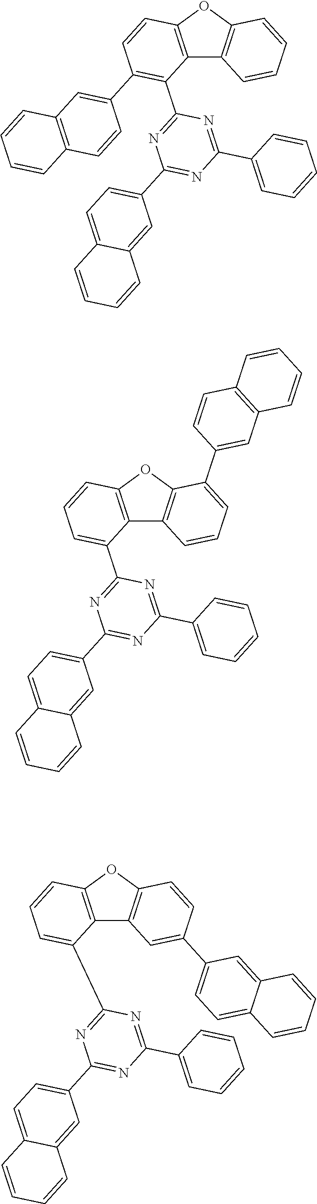 [SMILES: c1ccc(-c2nc(-c3ccc4ccccc4c3)nc(-c3c(-c4ccc5ccccc5c4)ccc4oc5ccccc5c34)n2)cc1 ; c1ccc(-c2nc(-c3ccc4ccccc4c3)nc(-c3cccc4oc5c(-c6ccc7ccccc7c6)cccc5c34)n2)cc1 ; c1ccc(-c2nc(-c3ccc4ccccc4c3)nc(-c3cccc4oc5ccc(-c6ccc7ccccc7c6)cc5c34)n2)cc1]

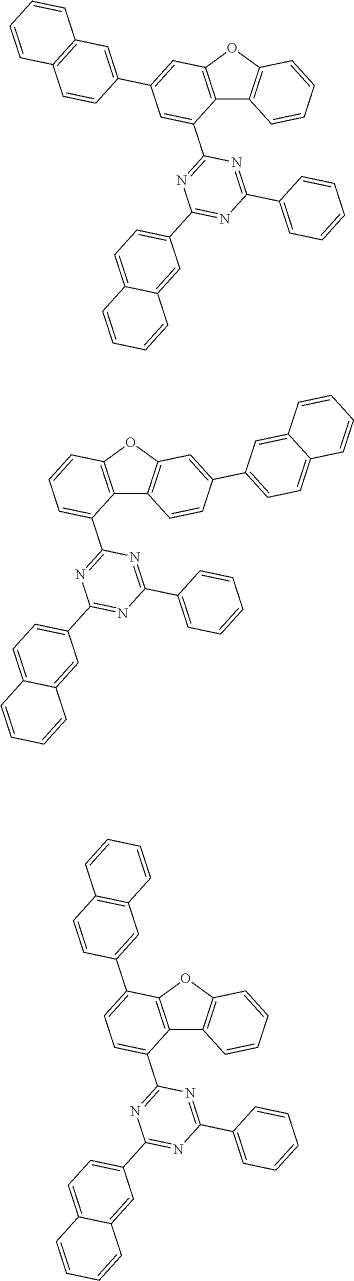 [SMILES: c1ccc(-c2nc(-c3ccc4ccccc4c3)nc(-c3cc(-c4ccc5ccccc5c4)cc4oc5ccccc5c34)n2)cc1 ; c1ccc(-c2nc(-c3ccc4ccccc4c3)nc(-c3cccc4oc5cc(-c6ccc7ccccc7c6)ccc5c34)n2)cc1 ; c1ccc(-c2nc(-c3ccc4ccccc4c3)nc(-c3ccc(-c4ccc5ccccc5c4)c4oc5ccccc5c34)n2)cc1]

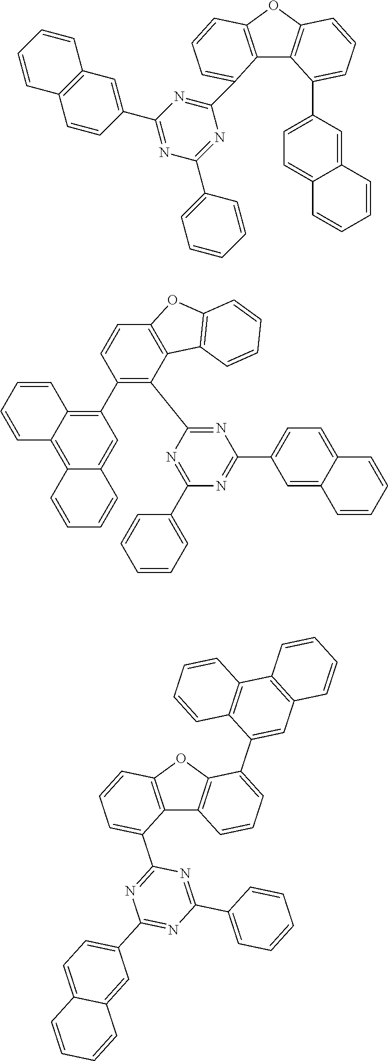

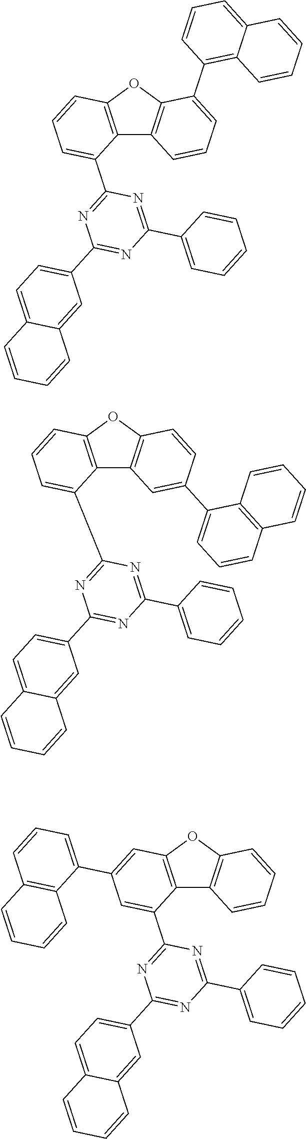

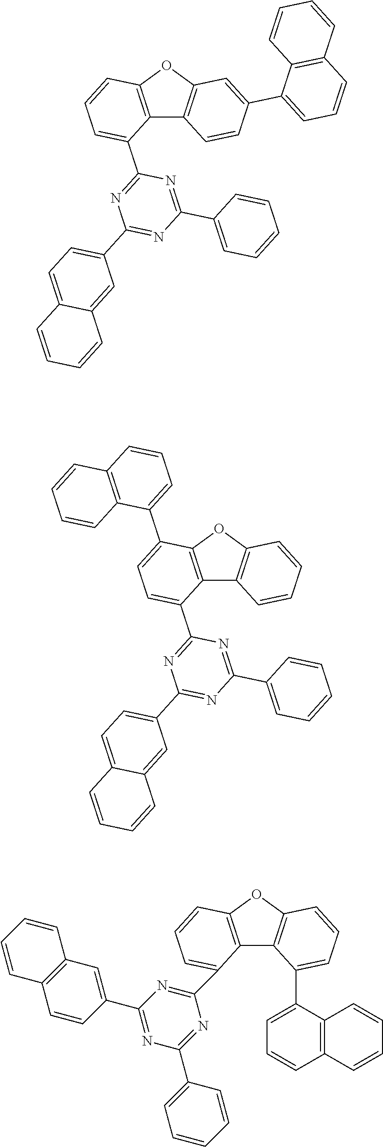 [SMILES: c1ccc(-c2nc(-c3ccc4ccccc4c3)nc(-c3cccc4oc5cc(-c6cccc7ccccc67)ccc5c34)n2)cc1 ; c1ccc(-c2nc(-c3ccc4ccccc4c3)nc(-c3ccc(-c4cccc5ccccc45)c4oc5ccccc5c34)n2)cc1 ; c1ccc(-c2nc(-c3ccc4ccccc4c3)nc(-c3cccc4oc5cccc(-c6cccc7ccccc67)c5c34)n2)cc1]

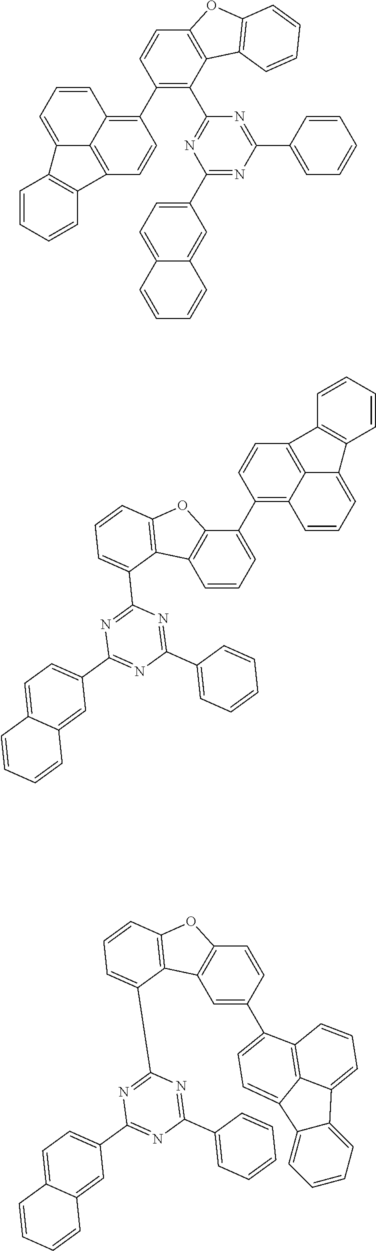 [SMILES: c1ccc(-c2nc(-c3ccc4ccccc4c3)nc(-c3c(-c4ccc5c6c(cccc46)-c4ccccc4-5)ccc4oc5ccccc5c34)n2)cc1 ; c1ccc(-c2nc(-c3ccc4ccccc4c3)nc(-c3cccc4oc5c(-c6ccc7c8c(cccc68)-c6ccccc6-7)cccc5c34)n2)cc1 ; c1ccc(-c2nc(-c3ccc4ccccc4c3)nc(-c3cccc4oc5ccc(-c6ccc7c8c(cccc68)-c6ccccc6-7)cc5c34)n2)cc1]

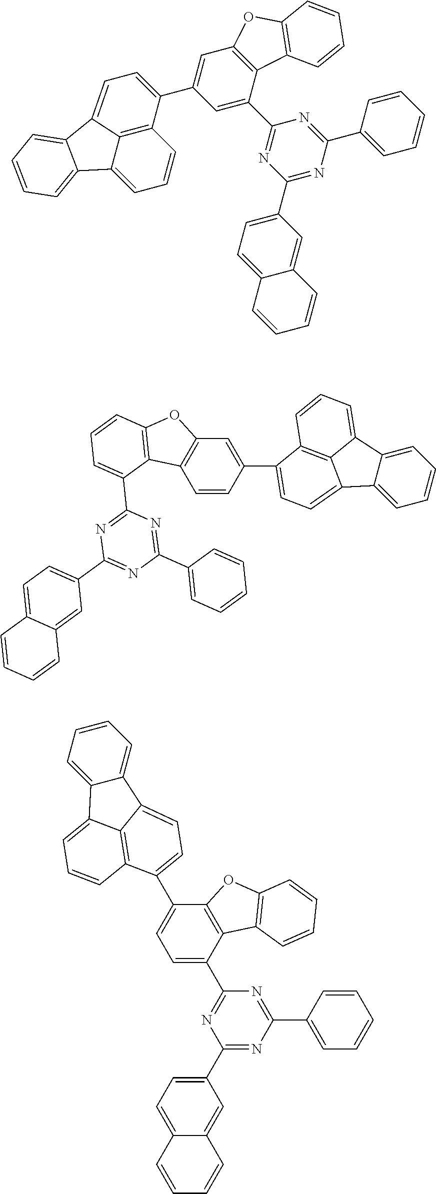 [SMILES: c1ccc(-c2nc(-c3ccc4ccccc4c3)nc(-c3cc(-c4ccc5c6c(cccc46)-c4ccccc4-5)cc4oc5ccccc5c34)n2)cc1 ; c1ccc(-c2nc(-c3ccc4ccccc4c3)nc(-c3cccc4oc5cc(-c6ccc7c8c(cccc68)-c6ccccc6-7)ccc5c34)n2)cc1 ; c1ccc(-c2nc(-c3ccc4ccccc4c3)nc(-c3ccc(-c4ccc5c6c(cccc46)-c4ccccc4-5)c4oc5ccccc5c34)n2)cc1]

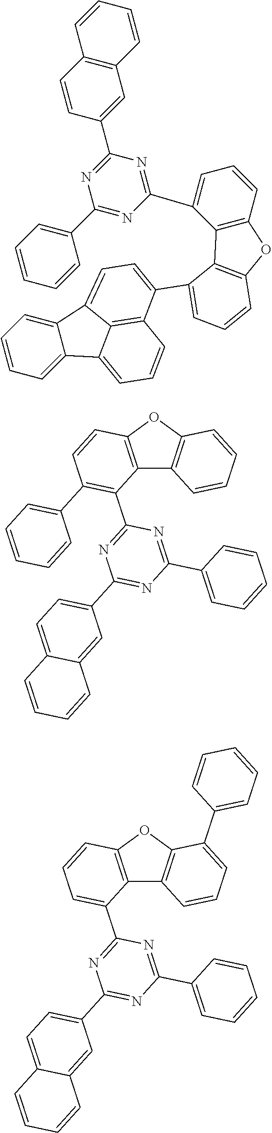

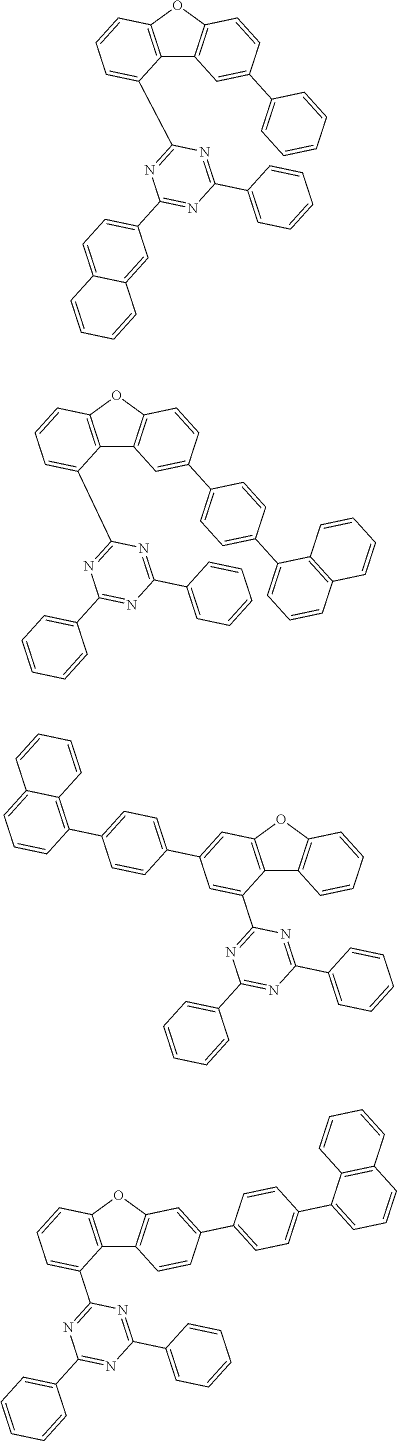 [SMILES: c1ccc(-c2ccc3oc4cccc(-c5nc(-c6ccccc6)nc(-c6ccc7ccccc7c6)n5)c4c3c2)cc1 ; c1ccc(-c2nc(-c3ccccc3)nc(-c3cccc4oc5ccc(-c6ccc(-c7cccc8ccccc78)cc6)cc5c34)n2)cc1 ; c1ccc(-c2nc(-c3ccccc3)nc(-c3cc(-c4ccc(-c5cccc6ccccc56)cc4)cc4oc5ccccc5c34)n2)cc1 ; c1ccc(-c2nc(-c3ccccc3)nc(-c3cccc4oc5cc(-c6ccc(-c7cccc8ccccc78)cc6)ccc5c34)n2)cc1]

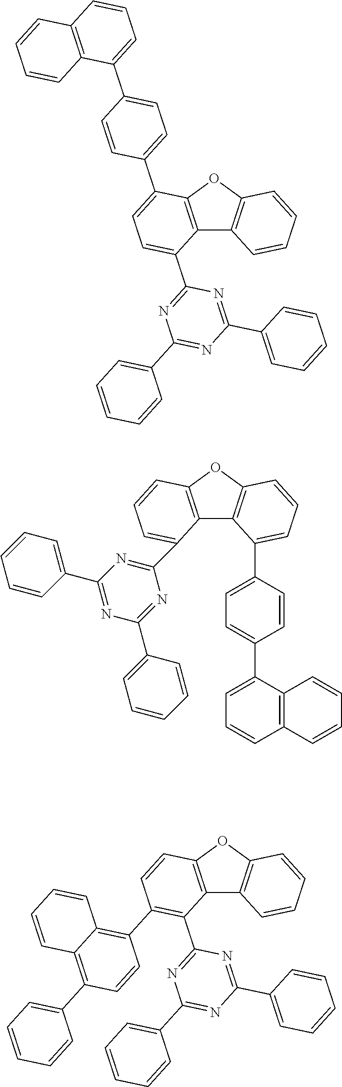 [SMILES: c1ccc(-c2nc(-c3ccccc3)nc(-c3ccc(-c4ccc(-c5cccc6ccccc56)cc4)c4oc5ccccc5c34)n2)cc1 ; c1ccc(-c2nc(-c3ccccc3)nc(-c3cccc4oc5cccc(-c6ccc(-c7cccc8ccccc78)cc6)c5c34)n2)cc1 ; c1ccc(-c2nc(-c3ccccc3)nc(-c3c(-c4ccc(-c5ccccc5)c5ccccc45)ccc4oc5ccccc5c34)n2)cc1]

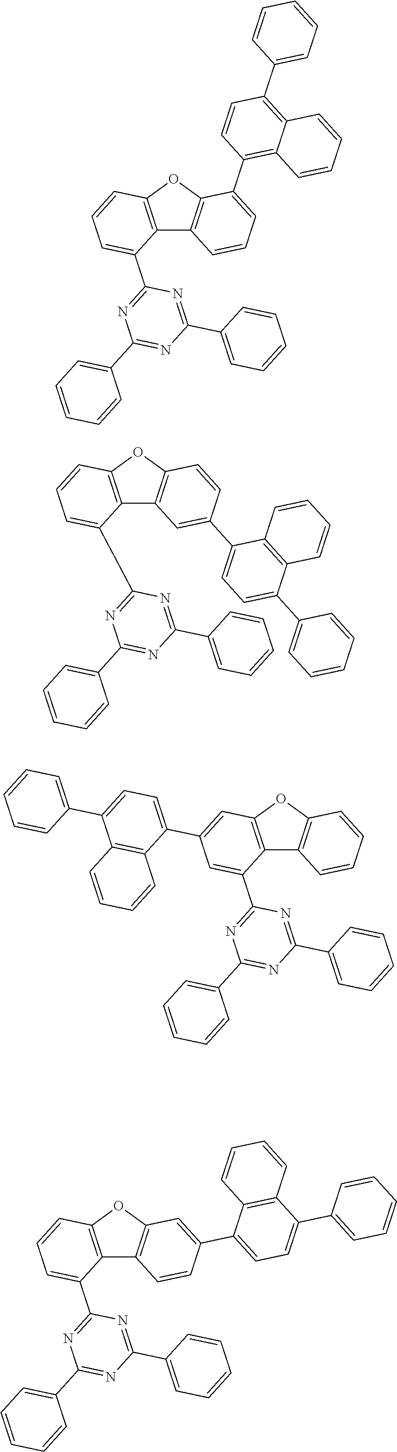

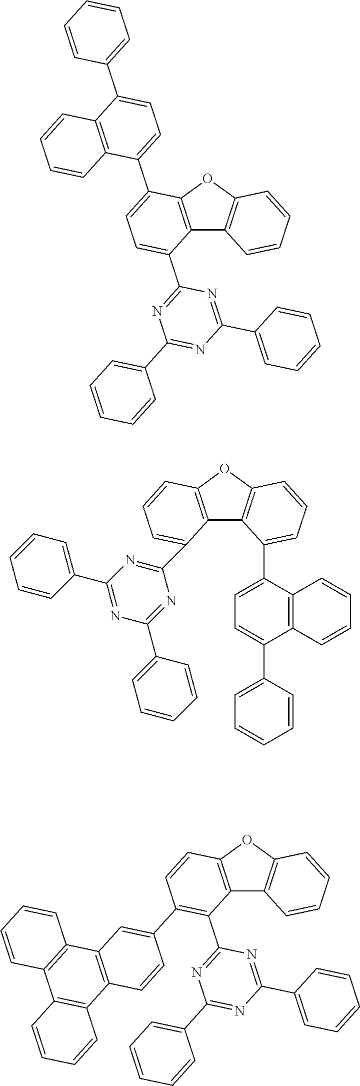 [SMILES: c1ccc(-c2nc(-c3ccccc3)nc(-c3ccc(-c4ccc(-c5ccccc5)c5ccccc45)c4oc5ccccc5c34)n2)cc1 ; c1ccc(-c2nc(-c3ccccc3)nc(-c3cccc4oc5cccc(-c6ccc(-c7ccccc7)c7ccccc67)c5c34)n2)cc1 ; c1ccc(-c2nc(-c3ccccc3)nc(-c3c(-c4ccc5c6ccccc6c6ccccc6c5c4)ccc4oc5ccccc5c34)n2)cc1]

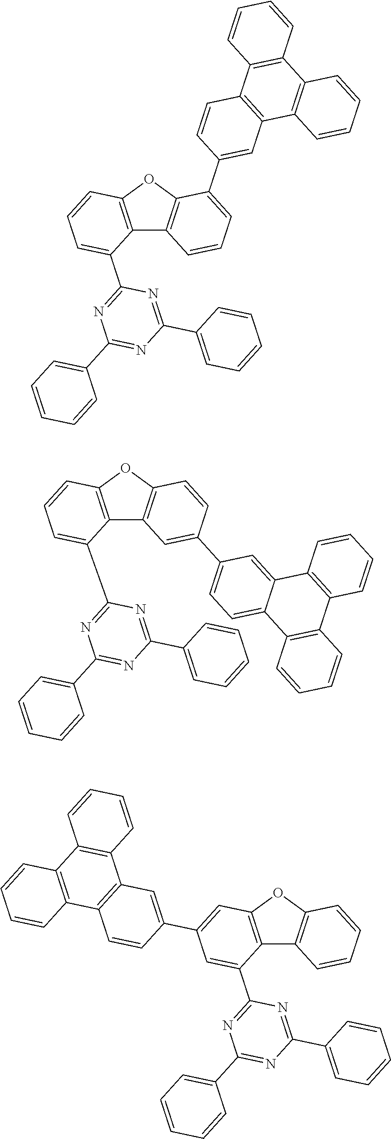 [SMILES: c1ccc(-c2nc(-c3ccccc3)nc(-c3cccc4oc5c(-c6ccc7c8ccccc8c8ccccc8c7c6)cccc5c34)n2)cc1 ; c1ccc(-c2nc(-c3ccccc3)nc(-c3cccc4oc5ccc(-c6ccc7c8ccccc8c8ccccc8c7c6)cc5c34)n2)cc1 ; c1ccc(-c2nc(-c3ccccc3)nc(-c3cc(-c4ccc5c6ccccc6c6ccccc6c5c4)cc4oc5ccccc5c34)n2)cc1]

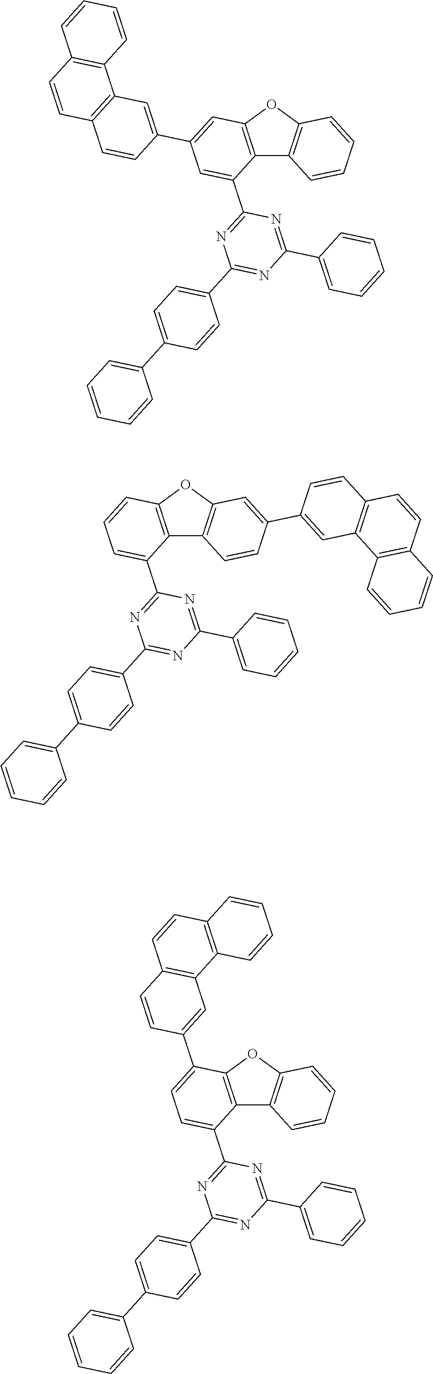 [SMILES: c1ccc(-c2ccc(-c3nc(-c4ccccc4)nc(-c4cc(-c5ccc6ccc7ccccc7c6c5)cc5oc6ccccc6c45)n3)cc2)cc1 ; c1ccc(-c2ccc(-c3nc(-c4ccccc4)nc(-c4cccc5oc6cc(-c7ccc8ccc9ccccc9c8c7)ccc6c45)n3)cc2)cc1 ; c1ccc(-c2ccc(-c3nc(-c4ccccc4)nc(-c4ccc(-c5ccc6ccc7ccccc7c6c5)c5oc6ccccc6c45)n3)cc2)cc1]

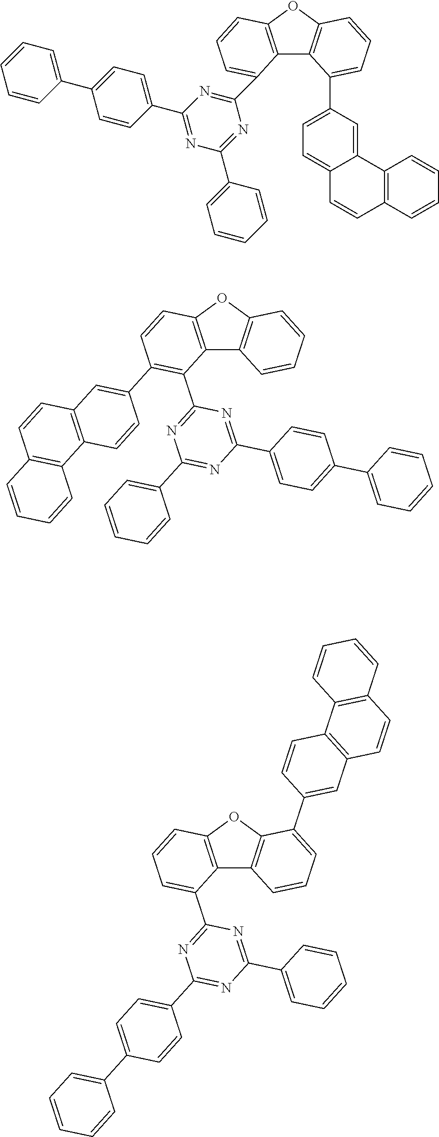 [SMILES: c1ccc(-c2ccc(-c3nc(-c4ccccc4)nc(-c4cccc5oc6cccc(-c7ccc8ccc9ccccc9c8c7)c6c45)n3)cc2)cc1 ; c1ccc(-c2ccc(-c3nc(-c4ccccc4)nc(-c4c(-c5ccc6c(ccc7ccccc76)c5)ccc5oc6ccccc6c45)n3)cc2)cc1 ; c1ccc(-c2ccc(-c3nc(-c4ccccc4)nc(-c4cccc5oc6c(-c7ccc8c(ccc9ccccc98)c7)cccc6c45)n3)cc2)cc1]

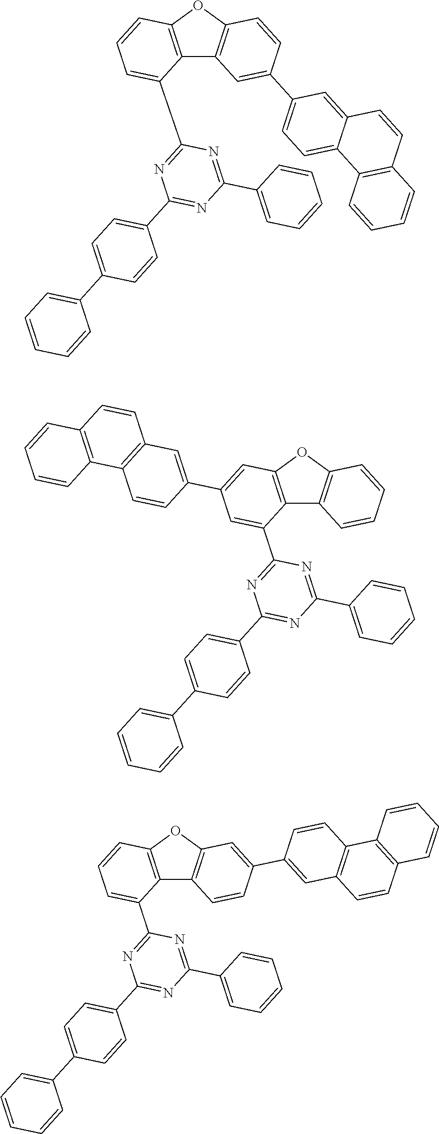 [SMILES: c1ccc(-c2ccc(-c3nc(-c4ccccc4)nc(-c4cccc5oc6ccc(-c7ccc8c(ccc9ccccc98)c7)cc6c45)n3)cc2)cc1 ; c1ccc(-c2ccc(-c3nc(-c4ccccc4)nc(-c4cc(-c5ccc6c(ccc7ccccc76)c5)cc5oc6ccccc6c45)n3)cc2)cc1 ; c1ccc(-c2ccc(-c3nc(-c4ccccc4)nc(-c4cccc5oc6cc(-c7ccc8c(ccc9ccccc98)c7)ccc6c45)n3)cc2)cc1]

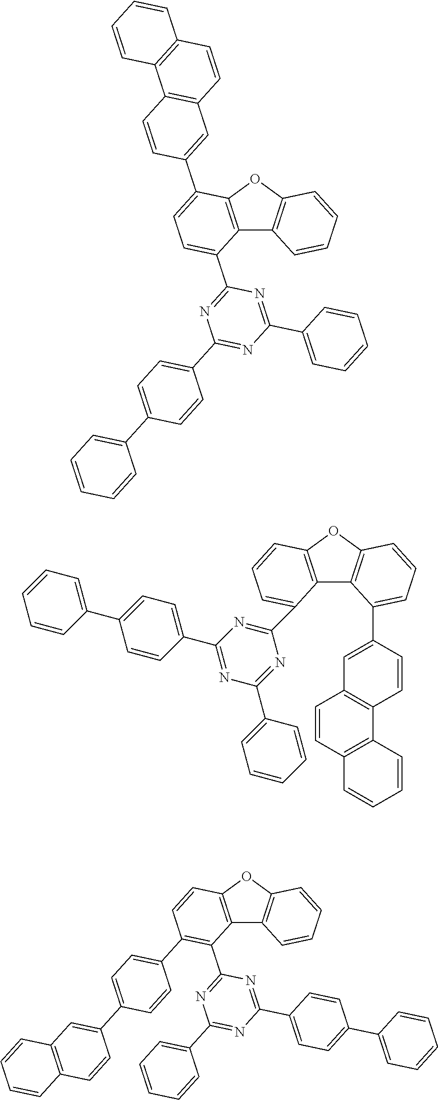 [SMILES: c1ccc(-c2ccc(-c3nc(-c4ccccc4)nc(-c4ccc(-c5ccc6c(ccc7ccccc76)c5)c5oc6ccccc6c45)n3)cc2)cc1 ; c1ccc(-c2ccc(-c3nc(-c4ccccc4)nc(-c4cccc5oc6cccc(-c7ccc8c(ccc9ccccc98)c7)c6c45)n3)cc2)cc1 ; c1ccc(-c2ccc(-c3nc(-c4ccccc4)nc(-c4c(-c5ccc(-c6ccc7ccccc7c6)cc5)ccc5oc6ccccc6c45)n3)cc2)cc1]

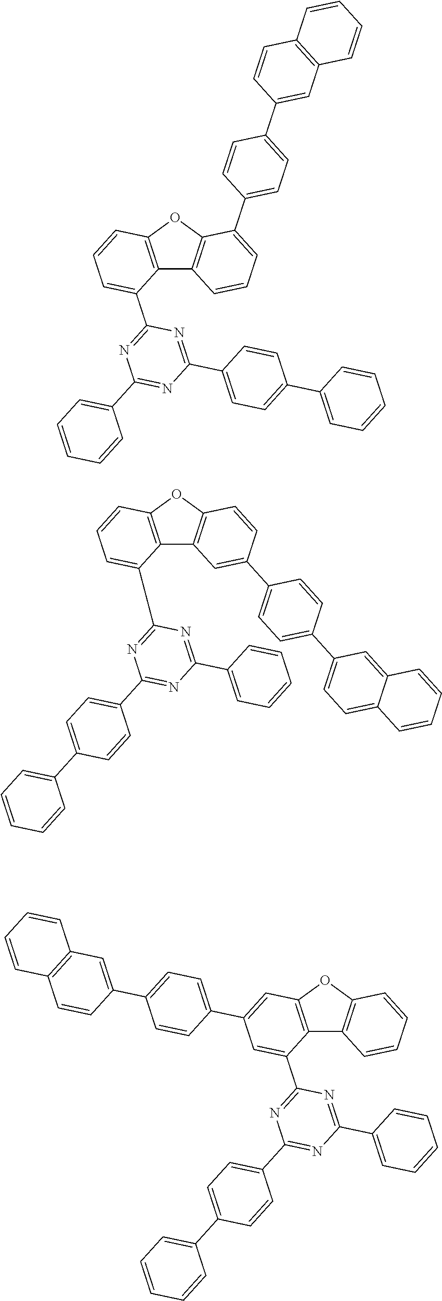 [SMILES: c1ccc(-c2ccc(-c3nc(-c4ccccc4)nc(-c4cccc5oc6c(-c7ccc(-c8ccc9ccccc9c8)cc7)cccc6c45)n3)cc2)cc1 ; c1ccc(-c2ccc(-c3nc(-c4ccccc4)nc(-c4cccc5oc6ccc(-c7ccc(-c8ccc9ccccc9c8)cc7)cc6c45)n3)cc2)cc1 ; c1ccc(-c2ccc(-c3nc(-c4ccccc4)nc(-c4cc(-c5ccc(-c6ccc7ccccc7c6)cc5)cc5oc6ccccc6c45)n3)cc2)cc1]

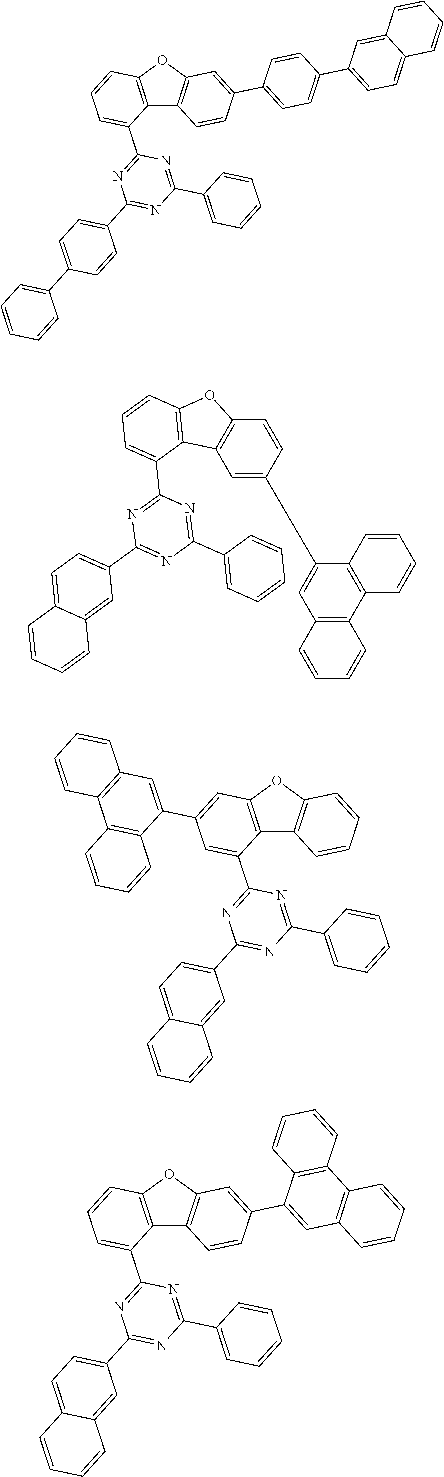 [SMILES: c1ccc(-c2ccc(-c3nc(-c4ccccc4)nc(-c4cccc5oc6cc(-c7ccc(-c8ccc9ccccc9c8)cc7)ccc6c45)n3)cc2)cc1 ; c1ccc(-c2nc(-c3ccc4ccccc4c3)nc(-c3cccc4oc5ccc(-c6cc7ccccc7c7ccccc67)cc5c34)n2)cc1 ; c1ccc(-c2nc(-c3ccc4ccccc4c3)nc(-c3cc(-c4cc5ccccc5c5ccccc45)cc4oc5ccccc5c34)n2)cc1 ; c1ccc(-c2nc(-c3ccc4ccccc4c3)nc(-c3cccc4oc5cc(-c6cc7ccccc7c7ccccc67)ccc5c34)n2)cc1]

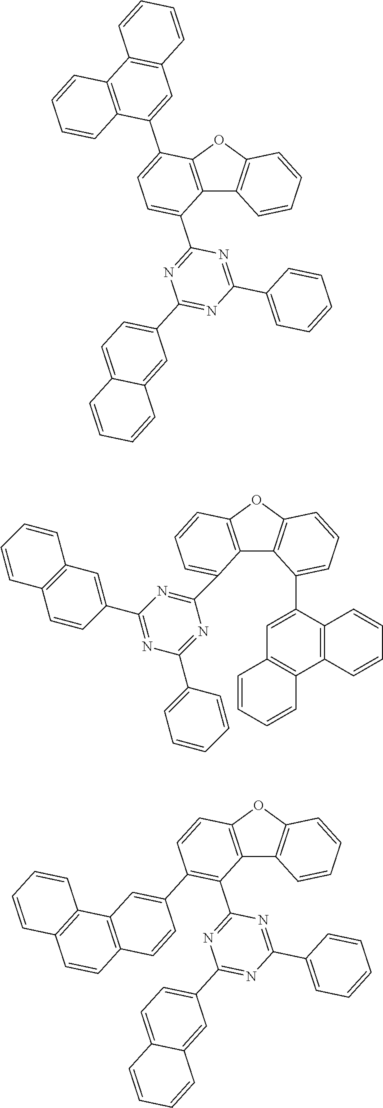 [SMILES: c1ccc(-c2nc(-c3ccc4ccccc4c3)nc(-c3ccc(-c4cc5ccccc5c5ccccc45)c4oc5ccccc5c34)n2)cc1 ; c1ccc(-c2nc(-c3ccc4ccccc4c3)nc(-c3cccc4oc5cccc(-c6cc7ccccc7c7ccccc67)c5c34)n2)cc1 ; c1ccc(-c2nc(-c3ccc4ccccc4c3)nc(-c3c(-c4ccc5ccc6ccccc6c5c4)ccc4oc5ccccc5c34)n2)cc1]

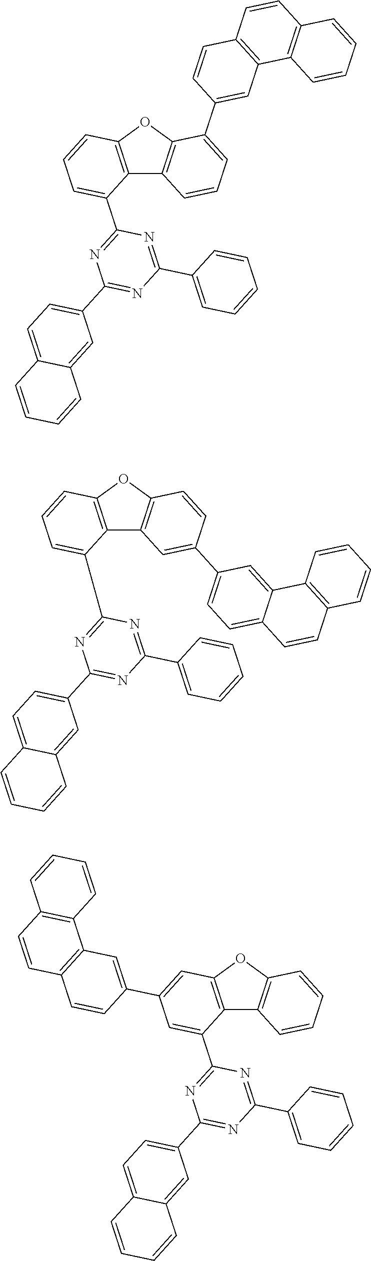

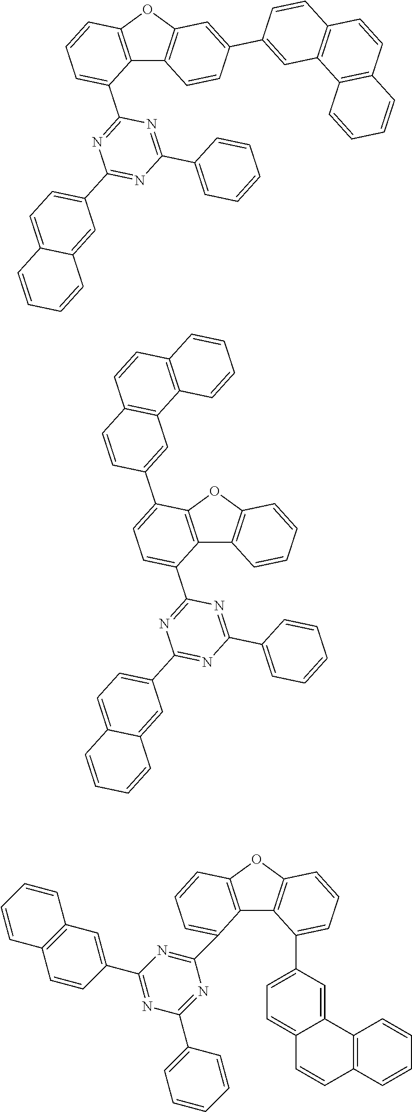 [SMILES: c1ccc(-c2nc(-c3ccc4ccccc4c3)nc(-c3cccc4oc5cc(-c6ccc7ccc8ccccc8c7c6)ccc5c34)n2)cc1 ; c1ccc(-c2nc(-c3ccc4ccccc4c3)nc(-c3ccc(-c4ccc5ccc6ccccc6c5c4)c4oc5ccccc5c34)n2)cc1 ; c1ccc(-c2nc(-c3ccc4ccccc4c3)nc(-c3cccc4oc5cccc(-c6ccc7ccc8ccccc8c7c6)c5c34)n2)cc1]

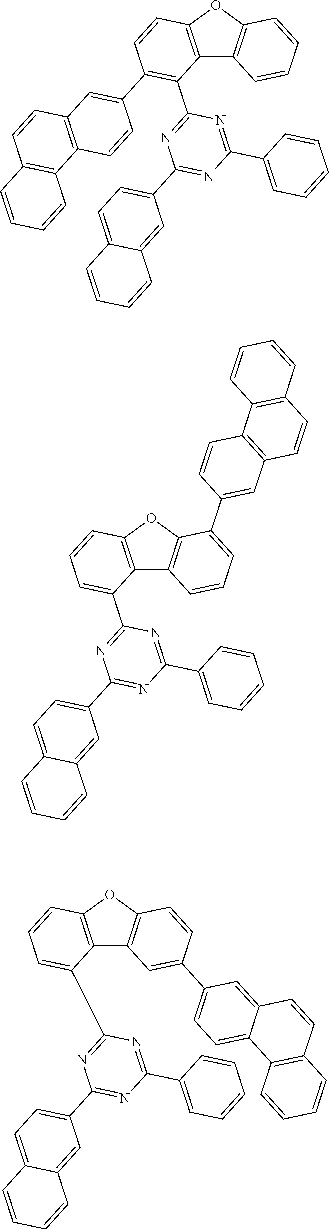 [SMILES: c1ccc(-c2nc(-c3ccc4ccccc4c3)nc(-c3c(-c4ccc5c(ccc6ccccc65)c4)ccc4oc5ccccc5c34)n2)cc1 ; c1ccc(-c2nc(-c3ccc4ccccc4c3)nc(-c3cccc4oc5c(-c6ccc7c(ccc8ccccc87)c6)cccc5c34)n2)cc1 ; c1ccc(-c2nc(-c3ccc4ccccc4c3)nc(-c3cccc4oc5ccc(-c6ccc7c(ccc8ccccc87)c6)cc5c34)n2)cc1]

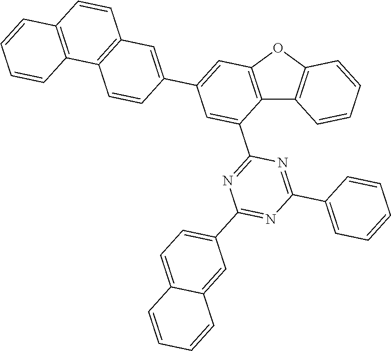 [SMILES: c1ccc(-c2nc(-c3ccc4ccccc4c3)nc(-c3cc(-c4ccc5c(ccc6ccccc65)c4)cc4oc5ccccc5c34)n2)cc1]

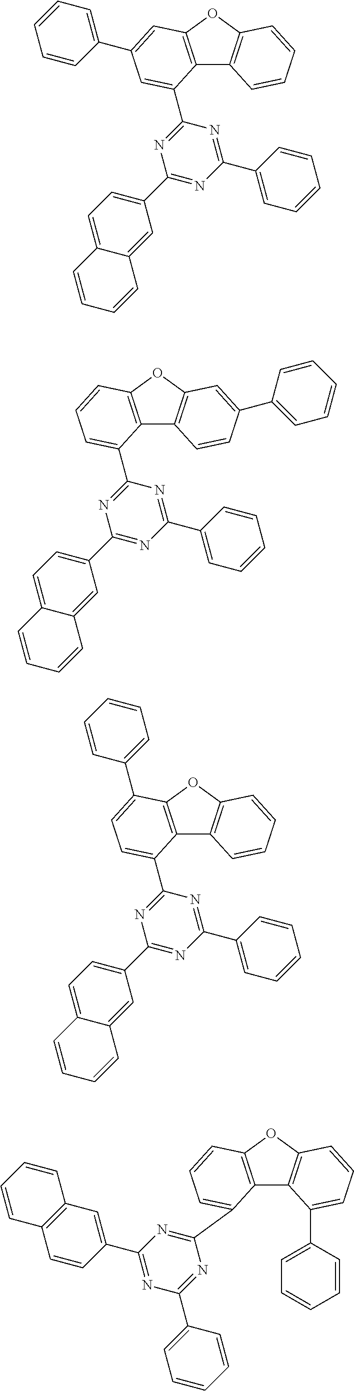 [SMILES: c1ccc(-c2cc(-c3nc(-c4ccccc4)nc(-c4ccc5ccccc5c4)n3)c3c(c2)oc2ccccc23)cc1 ; c1ccc(-c2ccc3c(c2)oc2cccc(-c4nc(-c5ccccc5)nc(-c5ccc6ccccc6c5)n4)c23)cc1 ; c1ccc(-c2nc(-c3ccc4ccccc4c3)nc(-c3ccc(-c4ccccc4)c4oc5ccccc5c34)n2)cc1 ; c1ccc(-c2nc(-c3ccc4ccccc4c3)nc(-c3cccc4oc5cccc(-c6ccccc6)c5c34)n2)cc1]

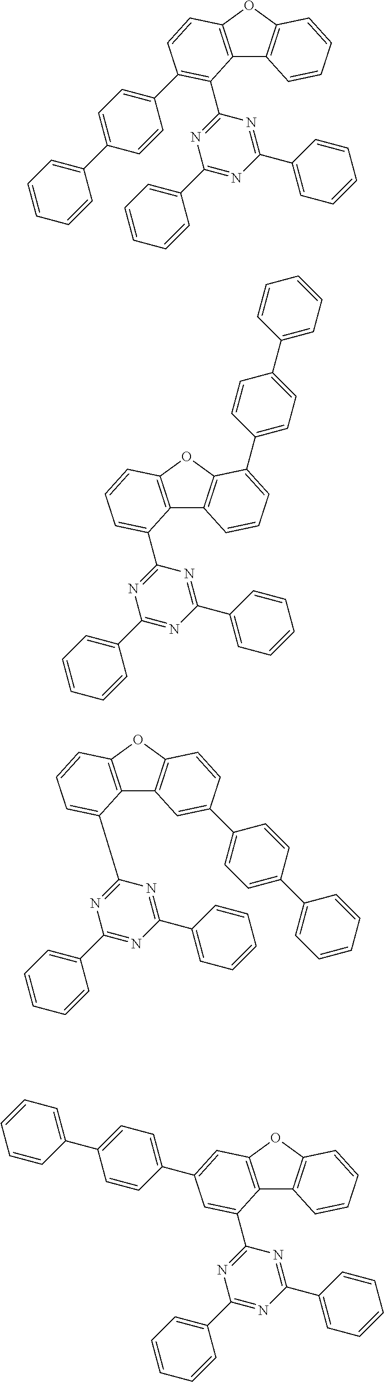 [SMILES: c1ccc(-c2ccc(-c3ccc4oc5ccccc5c4c3-c3nc(-c4ccccc4)nc(-c4ccccc4)n3)cc2)cc1 ; c1ccc(-c2ccc(-c3cccc4c3oc3cccc(-c5nc(-c6ccccc6)nc(-c6ccccc6)n5)c34)cc2)cc1 ; c1ccc(-c2ccc(-c3ccc4oc5cccc(-c6nc(-c7ccccc7)nc(-c7ccccc7)n6)c5c4c3)cc2)cc1 ; c1ccc(-c2ccc(-c3cc(-c4nc(-c5ccccc5)nc(-c5ccccc5)n4)c4c(c3)oc3ccccc34)cc2)cc1]

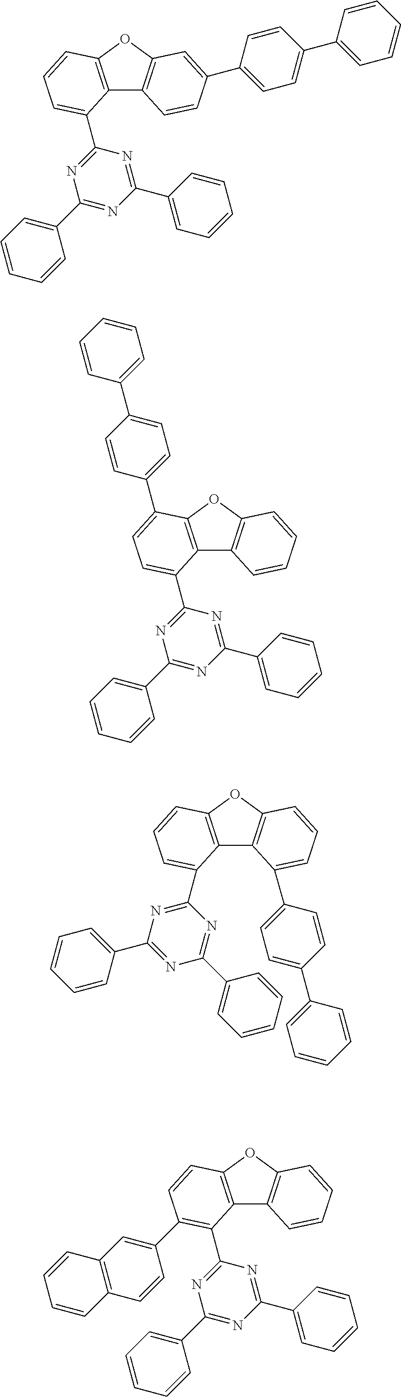 [SMILES: c1ccc(-c2ccc(-c3ccc4c(c3)oc3cccc(-c5nc(-c6ccccc6)nc(-c6ccccc6)n5)c34)cc2)cc1 ; c1ccc(-c2ccc(-c3ccc(-c4nc(-c5ccccc5)nc(-c5ccccc5)n4)c4c3oc3ccccc34)cc2)cc1 ; c1ccc(-c2ccc(-c3cccc4oc5cccc(-c6nc(-c7ccccc7)nc(-c7ccccc7)n6)c5c34)cc2)cc1 ; c1ccc(-c2nc(-c3ccccc3)nc(-c3c(-c4ccc5ccccc5c4)ccc4oc5ccccc5c34)n2)cc1]

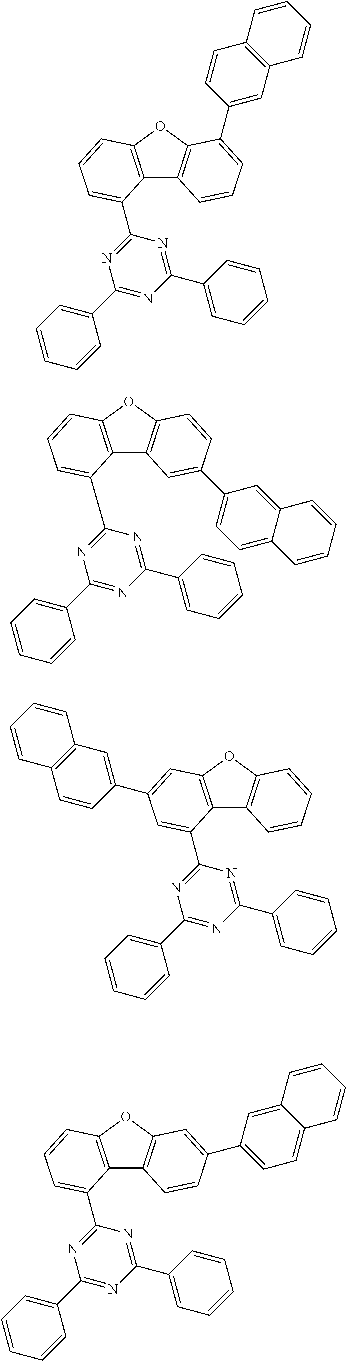

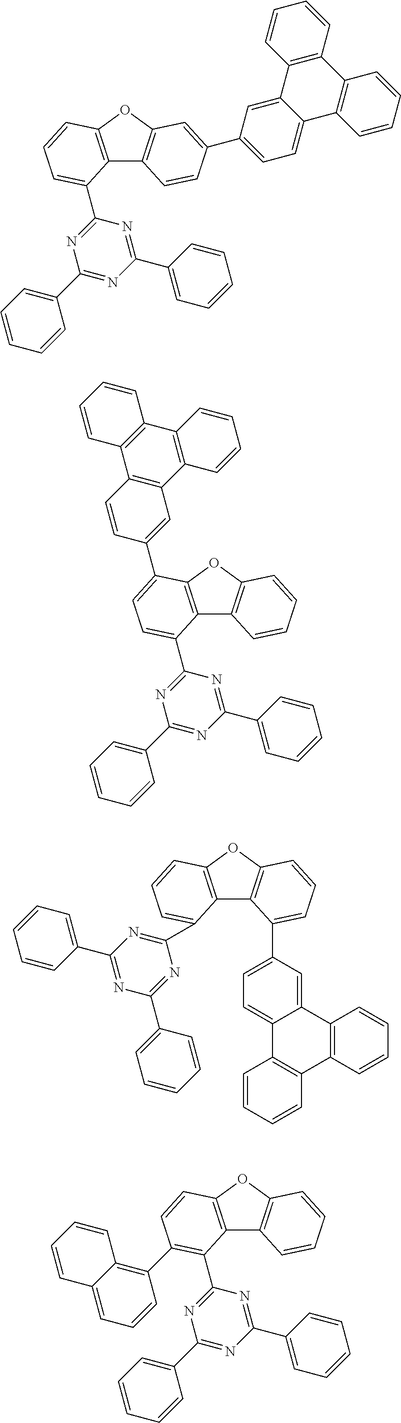 [SMILES: c1ccc(-c2nc(-c3ccccc3)nc(-c3cccc4oc5cc(-c6ccc7c8ccccc8c8ccccc8c7c6)ccc5c34)n2)cc1 ; c1ccc(-c2nc(-c3ccccc3)nc(-c3ccc(-c4ccc5c6ccccc6c6ccccc6c5c4)c4oc5ccccc5c34)n2)cc1 ; c1ccc(-c2nc(-c3ccccc3)nc(-c3cccc4oc5cccc(-c6ccc7c8ccccc8c8ccccc8c7c6)c5c34)n2)cc1 ; c1ccc(-c2nc(-c3ccccc3)nc(-c3c(-c4cccc5ccccc45)ccc4oc5ccccc5c34)n2)cc1]

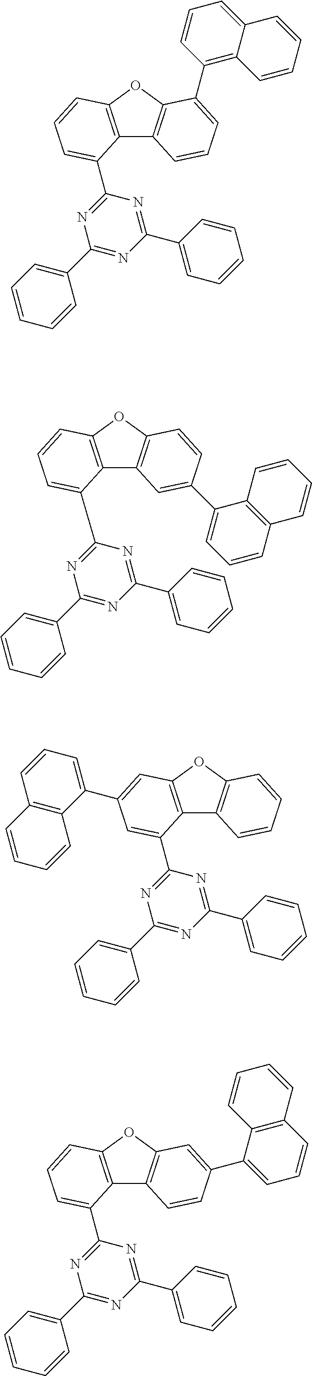 [SMILES: c1ccc(-c2nc(-c3ccccc3)nc(-c3cccc4oc5c(-c6cccc7ccccc67)cccc5c34)n2)cc1 ; c1ccc(-c2nc(-c3ccccc3)nc(-c3cccc4oc5ccc(-c6cccc7ccccc67)cc5c34)n2)cc1 ; c1ccc(-c2nc(-c3ccccc3)nc(-c3cc(-c4cccc5ccccc45)cc4oc5ccccc5c34)n2)cc1 ; c1ccc(-c2nc(-c3ccccc3)nc(-c3cccc4oc5cc(-c6cccc7ccccc67)ccc5c34)n2)cc1]

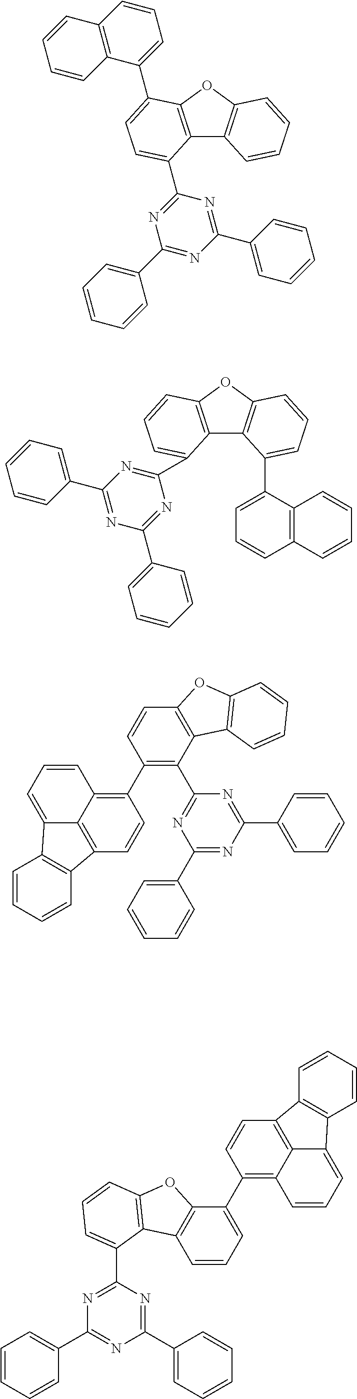 [SMILES: c1ccc(-c2nc(-c3ccccc3)nc(-c3ccc(-c4cccc5ccccc45)c4oc5ccccc5c34)n2)cc1 ; c1ccc(-c2nc(-c3ccccc3)nc(-c3cccc4oc5cccc(-c6cccc7ccccc67)c5c34)n2)cc1 ; c1ccc(-c2nc(-c3ccccc3)nc(-c3c(-c4ccc5c6c(cccc46)-c4ccccc4-5)ccc4oc5ccccc5c34)n2)cc1 ; c1ccc(-c2nc(-c3ccccc3)nc(-c3cccc4oc5c(-c6ccc7c8c(cccc68)-c6ccccc6-7)cccc5c34)n2)cc1]

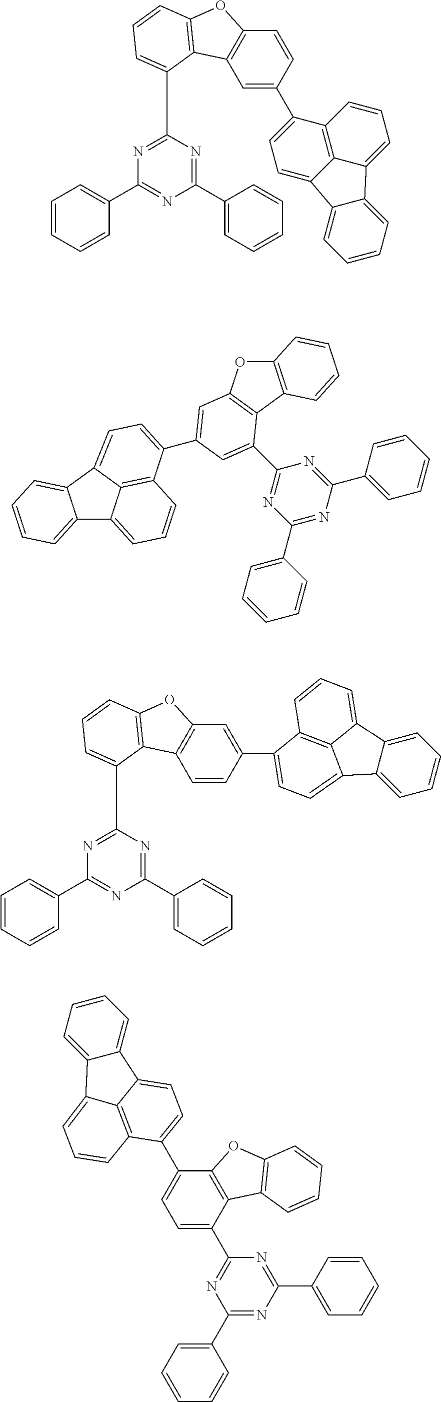 [SMILES: c1ccc(-c2nc(-c3ccccc3)nc(-c3cccc4oc5ccc(-c6ccc7c8c(cccc68)-c6ccccc6-7)cc5c34)n2)cc1 ; c1ccc(-c2nc(-c3ccccc3)nc(-c3cc(-c4ccc5c6c(cccc46)-c4ccccc4-5)cc4oc5ccccc5c34)n2)cc1 ; c1ccc(-c2nc(-c3ccccc3)nc(-c3cccc4oc5cc(-c6ccc7c8c(cccc68)-c6ccccc6-7)ccc5c34)n2)cc1 ; c1ccc(-c2nc(-c3ccccc3)nc(-c3ccc(-c4ccc5c6c(cccc46)-c4ccccc4-5)c4oc5ccccc5c34)n2)cc1]

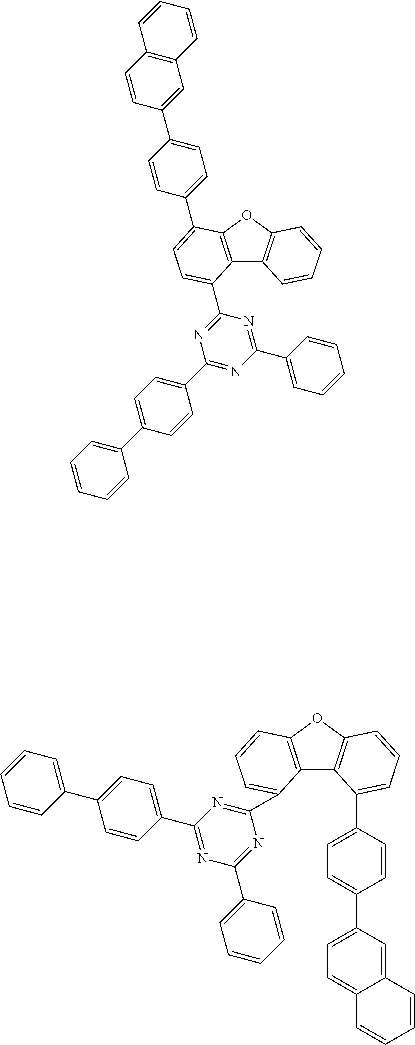 [SMILES: c1ccc(-c2ccc(-c3nc(-c4ccccc4)nc(-c4ccc(-c5ccc(-c6ccc7ccccc7c6)cc5)c5oc6ccccc6c45)n3)cc2)cc1 ; c1ccc(-c2ccc(-c3nc(-c4ccccc4)nc(-c4cccc5oc6cccc(-c7ccc(-c8ccc9ccccc9c8)cc7)c6c45)n3)cc2)cc1]

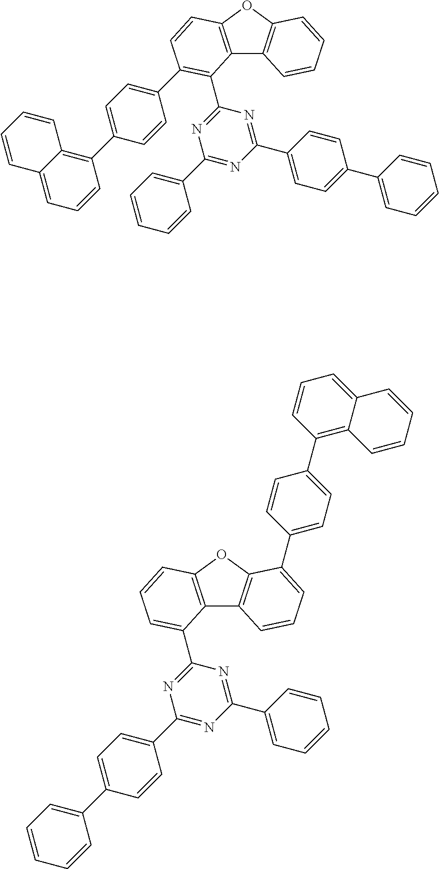 [SMILES: c1ccc(-c2ccc(-c3nc(-c4ccccc4)nc(-c4c(-c5ccc(-c6cccc7ccccc67)cc5)ccc5oc6ccccc6c45)n3)cc2)cc1 ; c1ccc(-c2ccc(-c3nc(-c4ccccc4)nc(-c4cccc5oc6c(-c7ccc(-c8cccc9ccccc89)cc7)cccc6c45)n3)cc2)cc1]

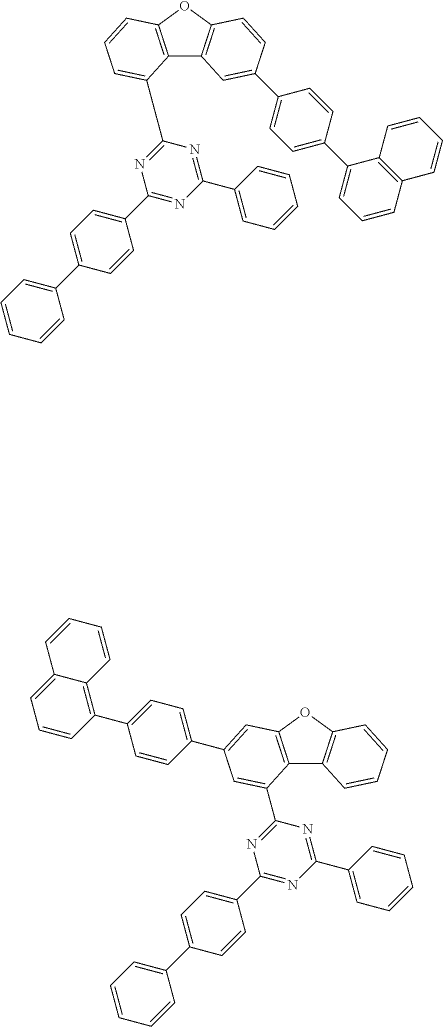 [SMILES: c1ccc(-c2ccc(-c3nc(-c4ccccc4)nc(-c4cccc5oc6ccc(-c7ccc(-c8cccc9ccccc89)cc7)cc6c45)n3)cc2)cc1 ; c1ccc(-c2ccc(-c3nc(-c4ccccc4)nc(-c4cc(-c5ccc(-c6cccc7ccccc67)cc5)cc5oc6ccccc6c45)n3)cc2)cc1]

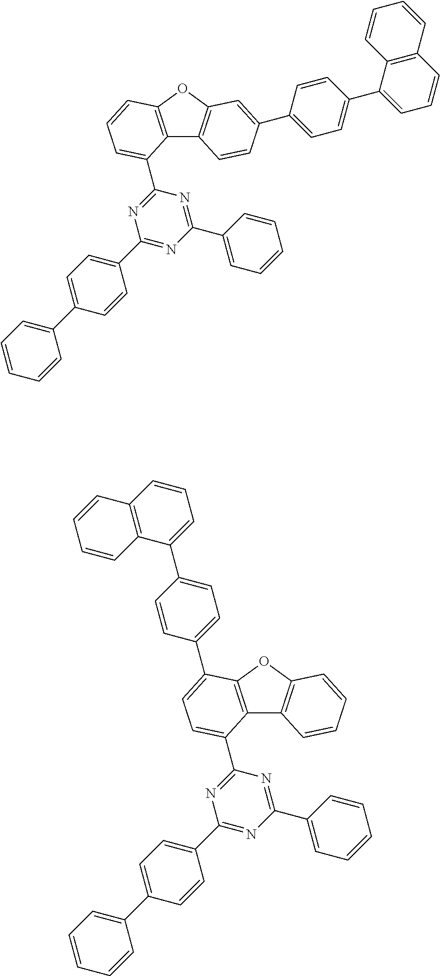 [SMILES: c1ccc(-c2ccc(-c3nc(-c4ccccc4)nc(-c4cccc5oc6cc(-c7ccc(-c8cccc9ccccc89)cc7)ccc6c45)n3)cc2)cc1 ; c1ccc(-c2ccc(-c3nc(-c4ccccc4)nc(-c4ccc(-c5ccc(-c6cccc7ccccc67)cc5)c5oc6ccccc6c45)n3)cc2)cc1]

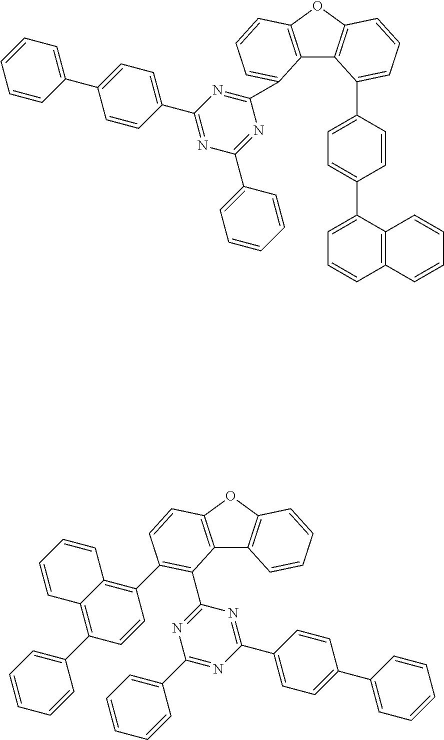 [SMILES: c1ccc(-c2ccc(-c3nc(-c4ccccc4)nc(-c4cccc5oc6cccc(-c7ccc(-c8cccc9ccccc89)cc7)c6c45)n3)cc2)cc1 ; c1ccc(-c2ccc(-c3nc(-c4ccccc4)nc(-c4c(-c5ccc(-c6ccccc6)c6ccccc56)ccc5oc6ccccc6c45)n3)cc2)cc1]

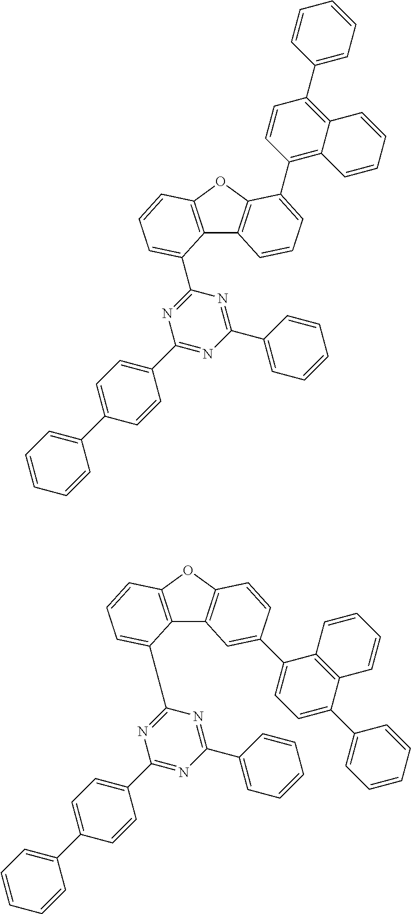 [SMILES: c1ccc(-c2ccc(-c3nc(-c4ccccc4)nc(-c4cccc5oc6c(-c7ccc(-c8ccccc8)c8ccccc78)cccc6c45)n3)cc2)cc1 ; c1ccc(-c2ccc(-c3nc(-c4ccccc4)nc(-c4cccc5oc6ccc(-c7ccc(-c8ccccc8)c8ccccc78)cc6c45)n3)cc2)cc1]

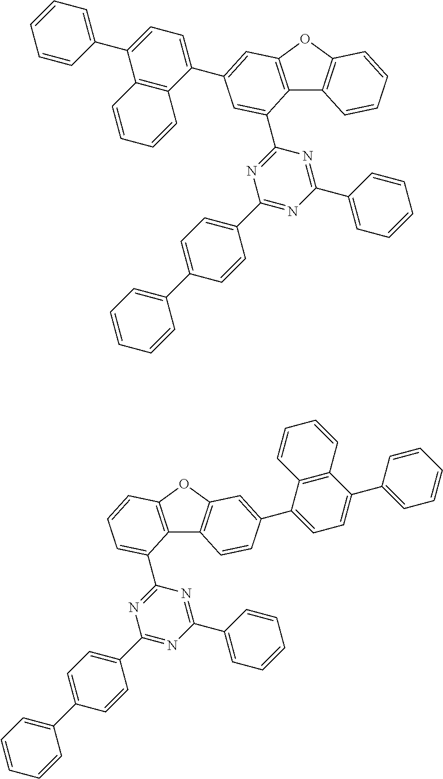 [SMILES: c1ccc(-c2ccc(-c3nc(-c4ccccc4)nc(-c4cc(-c5ccc(-c6ccccc6)c6ccccc56)cc5oc6ccccc6c45)n3)cc2)cc1 ; c1ccc(-c2ccc(-c3nc(-c4ccccc4)nc(-c4cccc5oc6cc(-c7ccc(-c8ccccc8)c8ccccc78)ccc6c45)n3)cc2)cc1]

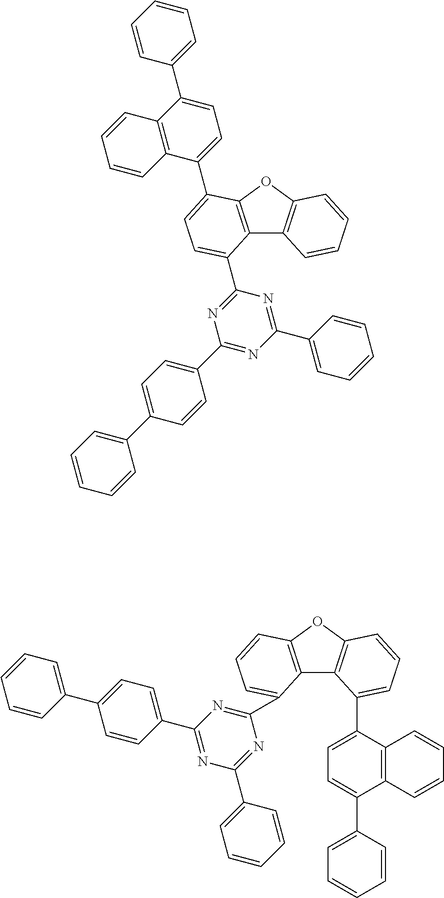 [SMILES: c1ccc(-c2ccc(-c3nc(-c4ccccc4)nc(-c4ccc(-c5ccc(-c6ccccc6)c6ccccc56)c5oc6ccccc6c45)n3)cc2)cc1 ; c1ccc(-c2ccc(-c3nc(-c4ccccc4)nc(-c4cccc5oc6cccc(-c7ccc(-c8ccccc8)c8ccccc78)c6c45)n3)cc2)cc1]

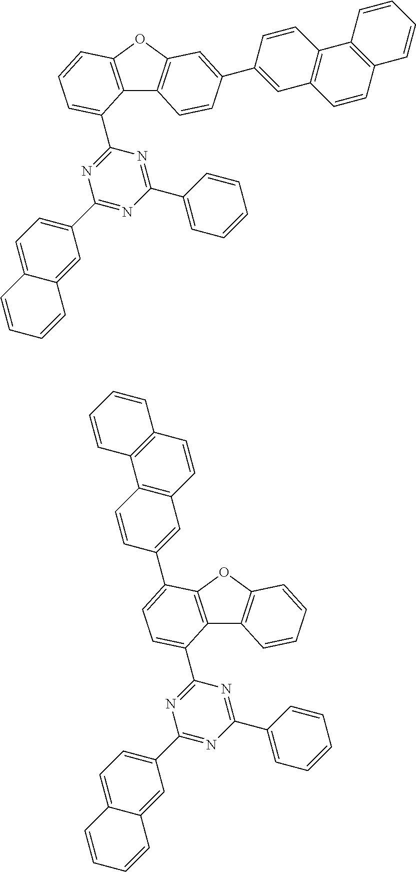 [SMILES: c1ccc(-c2nc(-c3ccc4ccccc4c3)nc(-c3cccc4oc5cc(-c6ccc7c(ccc8ccccc87)c6)ccc5c34)n2)cc1 ; c1ccc(-c2nc(-c3ccc4ccccc4c3)nc(-c3ccc(-c4ccc5c(ccc6ccccc65)c4)c4oc5ccccc5c34)n2)cc1]

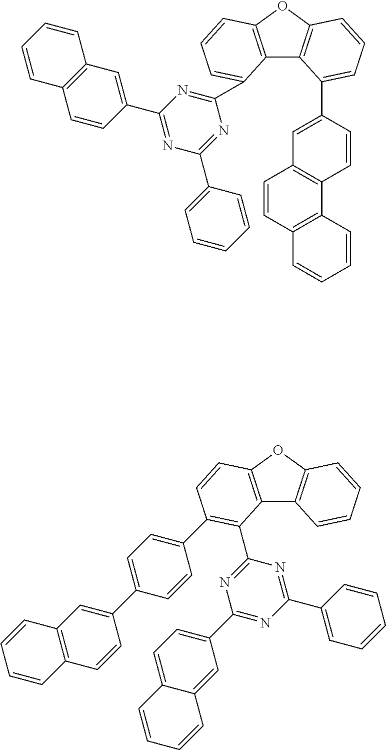 [SMILES: c1ccc(-c2nc(-c3ccc4ccccc4c3)nc(-c3cccc4oc5cccc(-c6ccc7c(ccc8ccccc87)c6)c5c34)n2)cc1 ; c1ccc(-c2nc(-c3ccc4ccccc4c3)nc(-c3c(-c4ccc(-c5ccc6ccccc6c5)cc4)ccc4oc5ccccc5c34)n2)cc1]

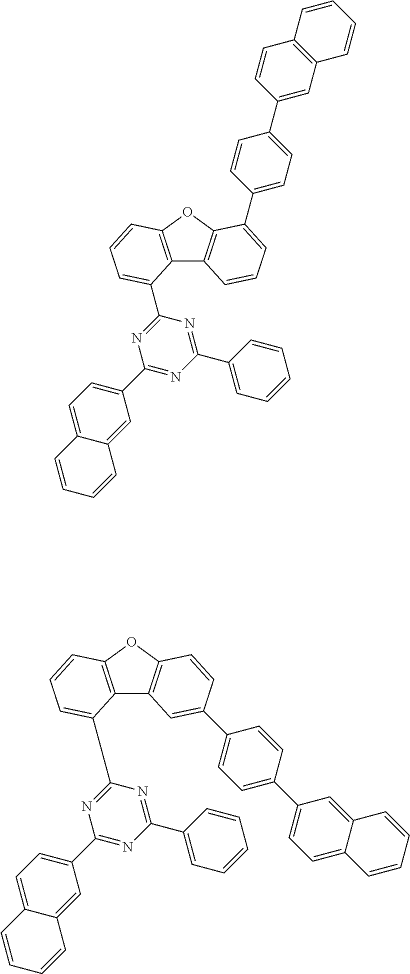 [SMILES: c1ccc(-c2nc(-c3ccc4ccccc4c3)nc(-c3cccc4oc5c(-c6ccc(-c7ccc8ccccc8c7)cc6)cccc5c34)n2)cc1 ; c1ccc(-c2nc(-c3ccc4ccccc4c3)nc(-c3cccc4oc5ccc(-c6ccc(-c7ccc8ccccc8c7)cc6)cc5c34)n2)cc1]

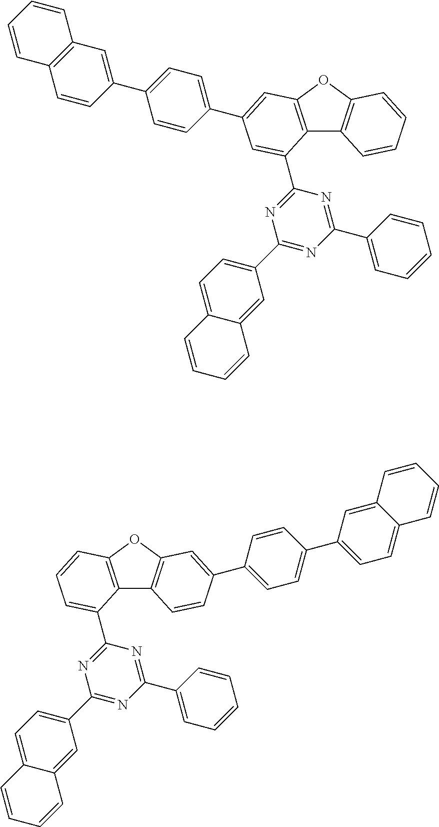 [SMILES: c1ccc(-c2nc(-c3ccc4ccccc4c3)nc(-c3cc(-c4ccc(-c5ccc6ccccc6c5)cc4)cc4oc5ccccc5c34)n2)cc1 ; c1ccc(-c2nc(-c3ccc4ccccc4c3)nc(-c3cccc4oc5cc(-c6ccc(-c7ccc8ccccc8c7)cc6)ccc5c34)n2)cc1]

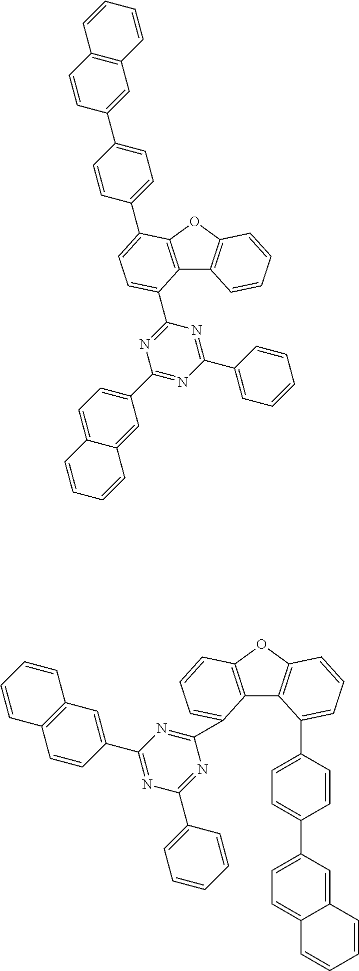 [SMILES: c1ccc(-c2nc(-c3ccc4ccccc4c3)nc(-c3ccc(-c4ccc(-c5ccc6ccccc6c5)cc4)c4oc5ccccc5c34)n2)cc1 ; c1ccc(-c2nc(-c3ccc4ccccc4c3)nc(-c3cccc4oc5cccc(-c6ccc(-c7ccc8ccccc8c7)cc6)c5c34)n2)cc1]

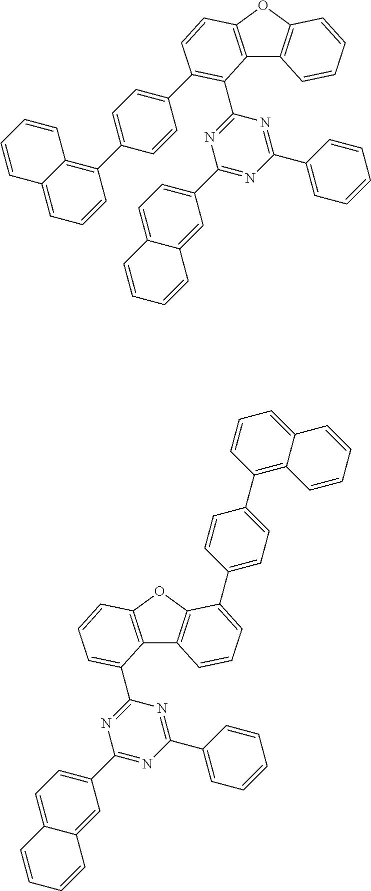 [SMILES: c1ccc(-c2nc(-c3ccc4ccccc4c3)nc(-c3c(-c4ccc(-c5cccc6ccccc56)cc4)ccc4oc5ccccc5c34)n2)cc1 ; c1ccc(-c2nc(-c3ccc4ccccc4c3)nc(-c3cccc4oc5c(-c6ccc(-c7cccc8ccccc78)cc6)cccc5c34)n2)cc1]

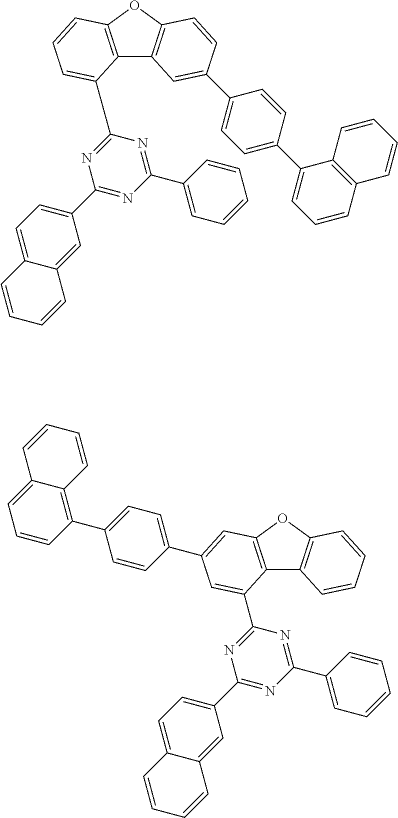 [SMILES: c1ccc(-c2nc(-c3ccc4ccccc4c3)nc(-c3cccc4oc5ccc(-c6ccc(-c7cccc8ccccc78)cc6)cc5c34)n2)cc1 ; c1ccc(-c2nc(-c3ccc4ccccc4c3)nc(-c3cc(-c4ccc(-c5cccc6ccccc56)cc4)cc4oc5ccccc5c34)n2)cc1]

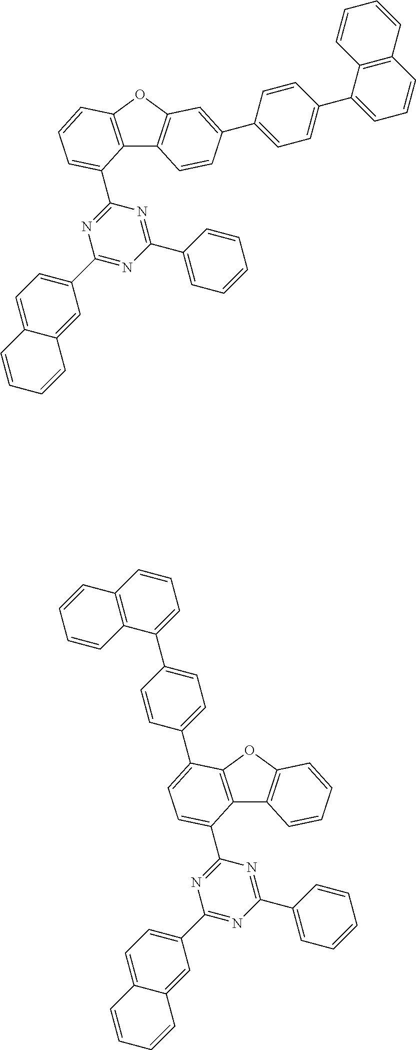 [SMILES: c1ccc(-c2nc(-c3ccc4ccccc4c3)nc(-c3cccc4oc5cc(-c6ccc(-c7cccc8ccccc78)cc6)ccc5c34)n2)cc1 ; c1ccc(-c2nc(-c3ccc4ccccc4c3)nc(-c3ccc(-c4ccc(-c5cccc6ccccc56)cc4)c4oc5ccccc5c34)n2)cc1]

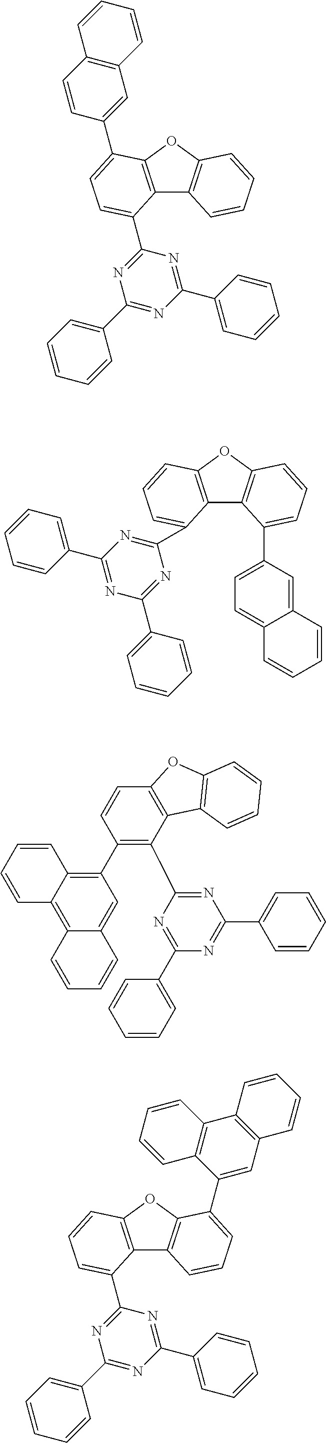 [SMILES: c1ccc(-c2nc(-c3ccccc3)nc(-c3ccc(-c4ccc5ccccc5c4)c4oc5ccccc5c34)n2)cc1 ; c1ccc(-c2nc(-c3ccccc3)nc(-c3cccc4oc5cccc(-c6ccc7ccccc7c6)c5c34)n2)cc1 ; c1ccc(-c2nc(-c3ccccc3)nc(-c3c(-c4cc5ccccc5c5ccccc45)ccc4oc5ccccc5c34)n2)cc1 ; c1ccc(-c2nc(-c3ccccc3)nc(-c3cccc4oc5c(-c6cc7ccccc7c7ccccc67)cccc5c34)n2)cc1]

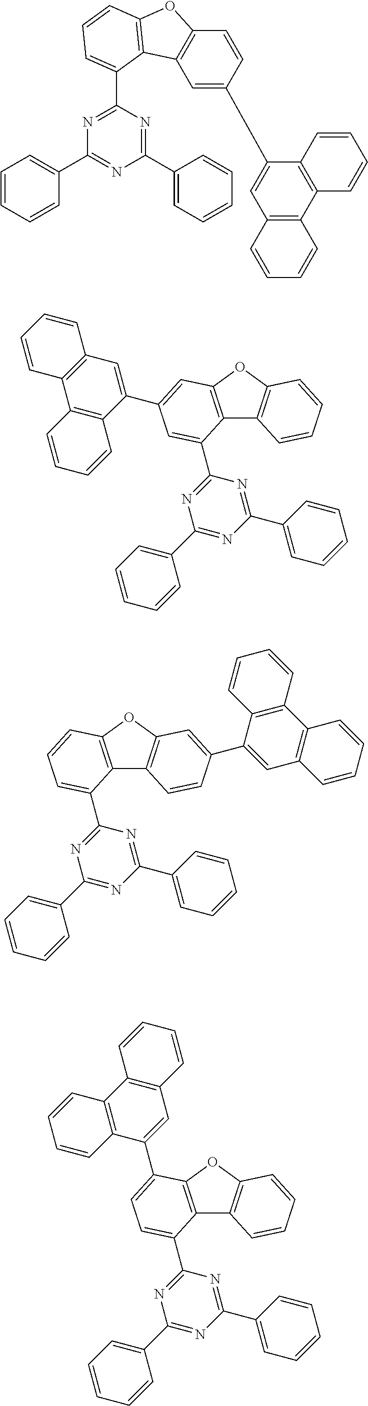 [SMILES: c1ccc(-c2nc(-c3ccccc3)nc(-c3cccc4oc5ccc(-c6cc7ccccc7c7ccccc67)cc5c34)n2)cc1 ; c1ccc(-c2nc(-c3ccccc3)nc(-c3cc(-c4cc5ccccc5c5ccccc45)cc4oc5ccccc5c34)n2)cc1 ; c1ccc(-c2nc(-c3ccccc3)nc(-c3cccc4oc5cc(-c6cc7ccccc7c7ccccc67)ccc5c34)n2)cc1 ; c1ccc(-c2nc(-c3ccccc3)nc(-c3ccc(-c4cc5ccccc5c5ccccc45)c4oc5ccccc5c34)n2)cc1]

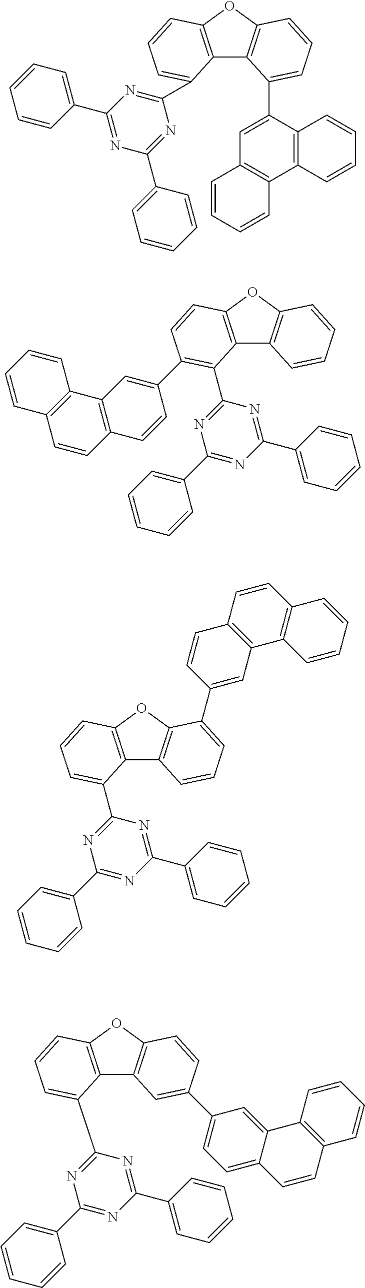 [SMILES: c1ccc(-c2nc(-c3ccccc3)nc(-c3cccc4oc5cccc(-c6cc7ccccc7c7ccccc67)c5c34)n2)cc1 ; c1ccc(-c2nc(-c3ccccc3)nc(-c3c(-c4ccc5ccc6ccccc6c5c4)ccc4oc5ccccc5c34)n2)cc1 ; c1ccc(-c2nc(-c3ccccc3)nc(-c3cccc4oc5c(-c6ccc7ccc8ccccc8c7c6)cccc5c34)n2)cc1 ; c1ccc(-c2nc(-c3ccccc3)nc(-c3cccc4oc5ccc(-c6ccc7ccc8ccccc8c7c6)cc5c34)n2)cc1]

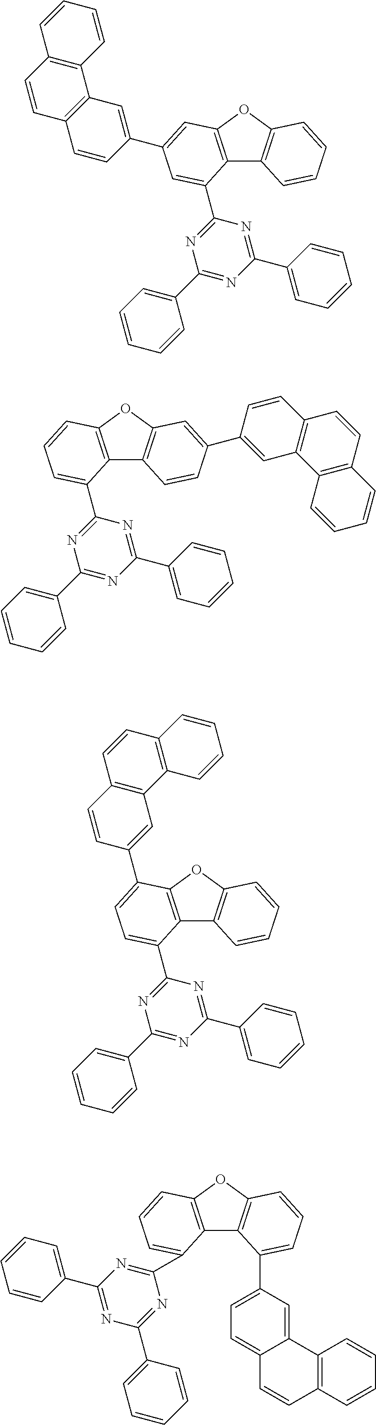 [SMILES: c1ccc(-c2nc(-c3ccccc3)nc(-c3cc(-c4ccc5ccc6ccccc6c5c4)cc4oc5ccccc5c34)n2)cc1 ; c1ccc(-c2nc(-c3ccccc3)nc(-c3cccc4oc5cc(-c6ccc7ccc8ccccc8c7c6)ccc5c34)n2)cc1 ; c1ccc(-c2nc(-c3ccccc3)nc(-c3ccc(-c4ccc5ccc6ccccc6c5c4)c4oc5ccccc5c34)n2)cc1 ; c1ccc(-c2nc(-c3ccccc3)nc(-c3cccc4oc5cccc(-c6ccc7ccc8ccccc8c7c6)c5c34)n2)cc1]

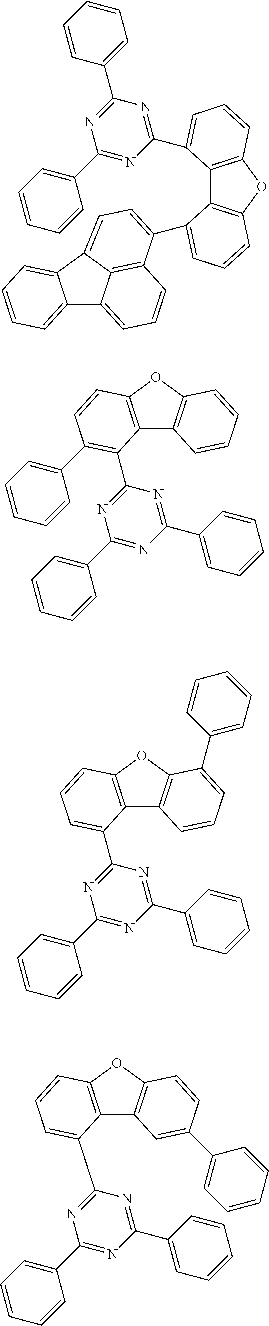 [SMILES: c1ccc(-c2nc(-c3ccccc3)nc(-c3cccc4oc5cccc(-c6ccc7c8c(cccc68)-c6ccccc6-7)c5c34)n2)cc1 ; c1ccc(-c2nc(-c3ccccc3)nc(-c3c(-c4ccccc4)ccc4oc5ccccc5c34)n2)cc1 ; c1ccc(-c2nc(-c3ccccc3)nc(-c3cccc4oc5c(-c6ccccc6)cccc5c34)n2)cc1 ; c1ccc(-c2ccc3oc4cccc(-c5nc(-c6ccccc6)nc(-c6ccccc6)n5)c4c3c2)cc1]

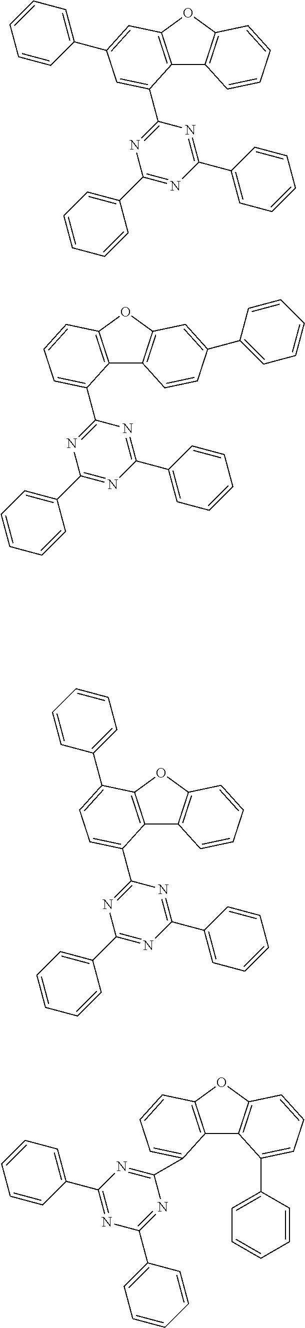 [SMILES: c1ccc(-c2cc(-c3nc(-c4ccccc4)nc(-c4ccccc4)n3)c3c(c2)oc2ccccc23)cc1 ; c1ccc(-c2ccc3c(c2)oc2cccc(-c4nc(-c5ccccc5)nc(-c5ccccc5)n4)c23)cc1 ; c1ccc(-c2nc(-c3ccccc3)nc(-c3ccc(-c4ccccc4)c4oc5ccccc5c34)n2)cc1 ; c1ccc(-c2nc(-c3ccccc3)nc(-c3cccc4oc5cccc(-c6ccccc6)c5c34)n2)cc1]

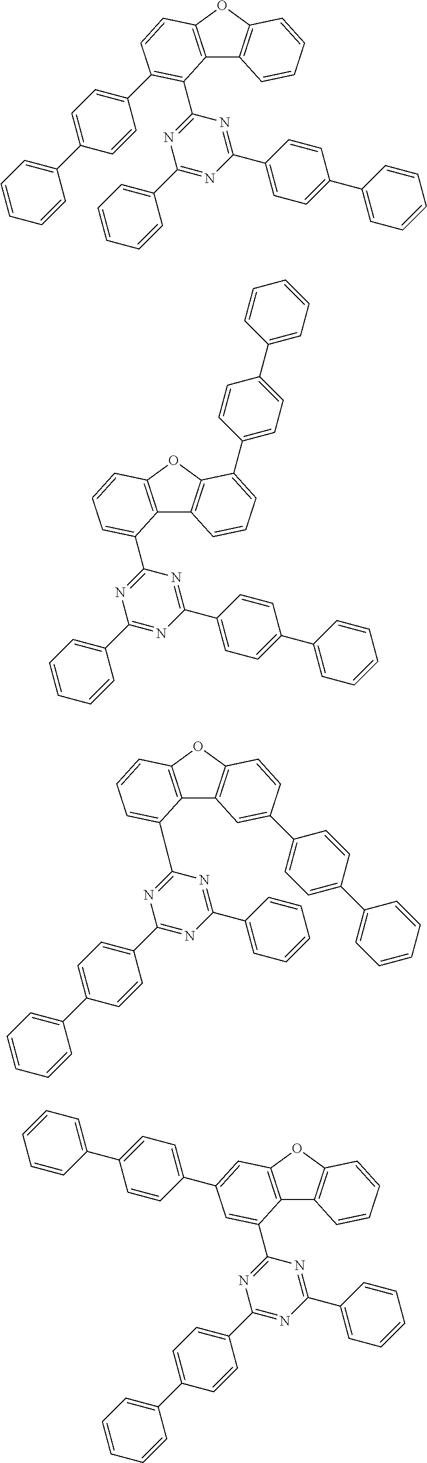

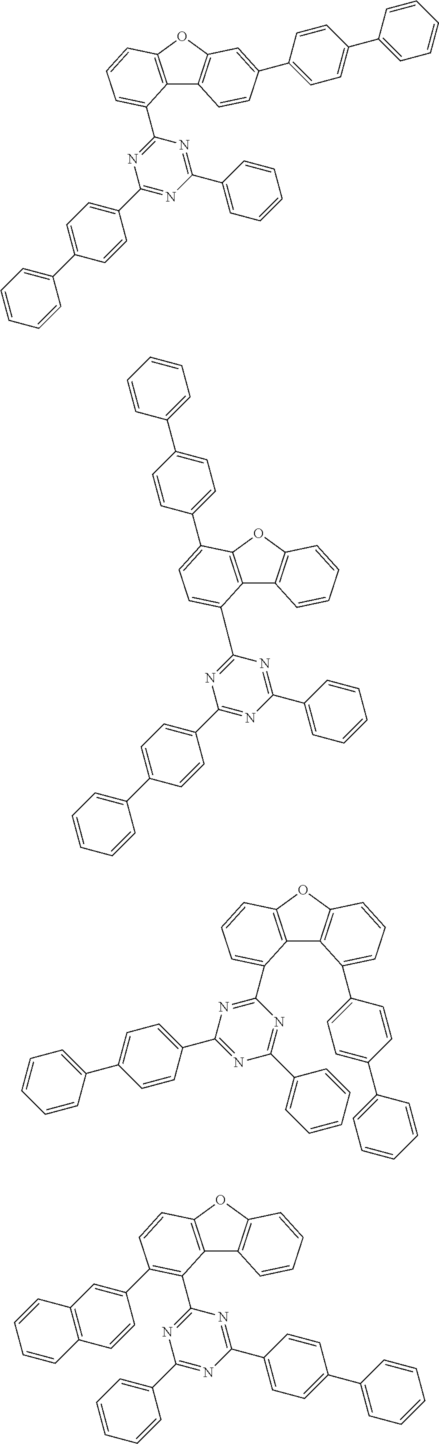 [SMILES: c1ccc(-c2ccc(-c3ccc4c(c3)oc3cccc(-c5nc(-c6ccccc6)nc(-c6ccc(-c7ccccc7)cc6)n5)c34)cc2)cc1 ; c1ccc(-c2ccc(-c3nc(-c4ccccc4)nc(-c4ccc(-c5ccc(-c6ccccc6)cc5)c5oc6ccccc6c45)n3)cc2)cc1 ; c1ccc(-c2ccc(-c3nc(-c4ccccc4)nc(-c4cccc5oc6cccc(-c7ccc(-c8ccccc8)cc7)c6c45)n3)cc2)cc1 ; c1ccc(-c2ccc(-c3nc(-c4ccccc4)nc(-c4c(-c5ccc6ccccc6c5)ccc5oc6ccccc6c45)n3)cc2)cc1]

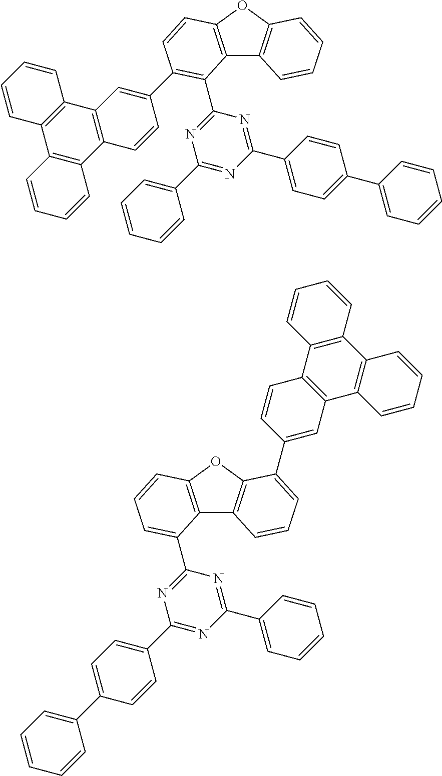 [SMILES: c1ccc(-c2ccc(-c3nc(-c4ccccc4)nc(-c4c(-c5ccc6c7ccccc7c7ccccc7c6c5)ccc5oc6ccccc6c45)n3)cc2)cc1 ; c1ccc(-c2ccc(-c3nc(-c4ccccc4)nc(-c4cccc5oc6c(-c7ccc8c9ccccc9c9ccccc9c8c7)cccc6c45)n3)cc2)cc1]

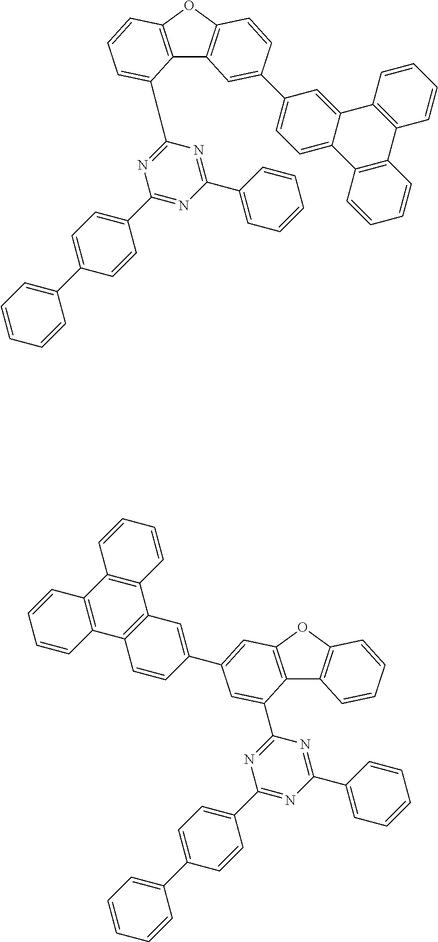 [SMILES: c1ccc(-c2ccc(-c3nc(-c4ccccc4)nc(-c4cccc5oc6ccc(-c7ccc8c9ccccc9c9ccccc9c8c7)cc6c45)n3)cc2)cc1 ; c1ccc(-c2ccc(-c3nc(-c4ccccc4)nc(-c4cc(-c5ccc6c7ccccc7c7ccccc7c6c5)cc5oc6ccccc6c45)n3)cc2)cc1]

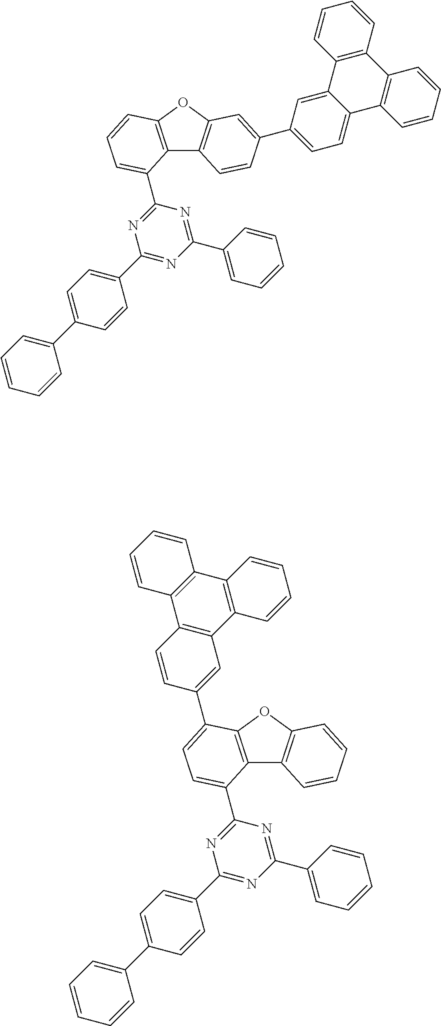 [SMILES: c1ccc(-c2ccc(-c3nc(-c4ccccc4)nc(-c4cccc5oc6cc(-c7ccc8c9ccccc9c9ccccc9c8c7)ccc6c45)n3)cc2)cc1 ; c1ccc(-c2ccc(-c3nc(-c4ccccc4)nc(-c4ccc(-c5ccc6c7ccccc7c7ccccc7c6c5)c5oc6ccccc6c45)n3)cc2)cc1]

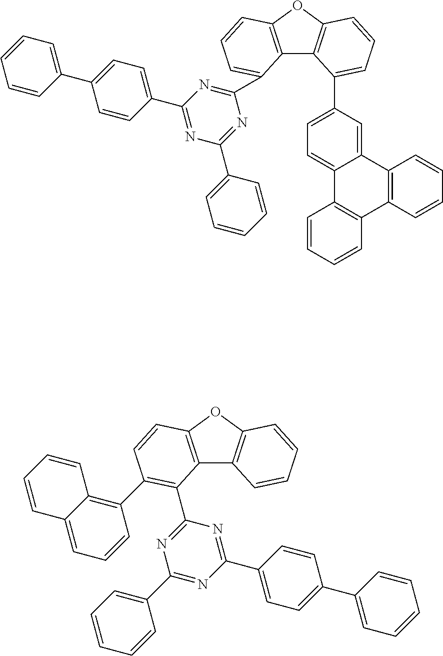 [SMILES: c1ccc(-c2ccc(-c3nc(-c4ccccc4)nc(-c4cccc5oc6cccc(-c7ccc8c9ccccc9c9ccccc9c8c7)c6c45)n3)cc2)cc1 ; c1ccc(-c2ccc(-c3nc(-c4ccccc4)nc(-c4c(-c5cccc6ccccc56)ccc5oc6ccccc6c45)n3)cc2)cc1]

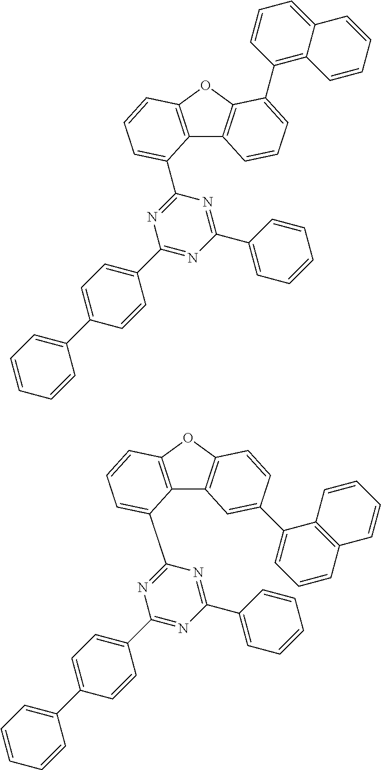 [SMILES: c1ccc(-c2ccc(-c3nc(-c4ccccc4)nc(-c4cccc5oc6c(-c7cccc8ccccc78)cccc6c45)n3)cc2)cc1 ; c1ccc(-c2ccc(-c3nc(-c4ccccc4)nc(-c4cccc5oc6ccc(-c7cccc8ccccc78)cc6c45)n3)cc2)cc1]

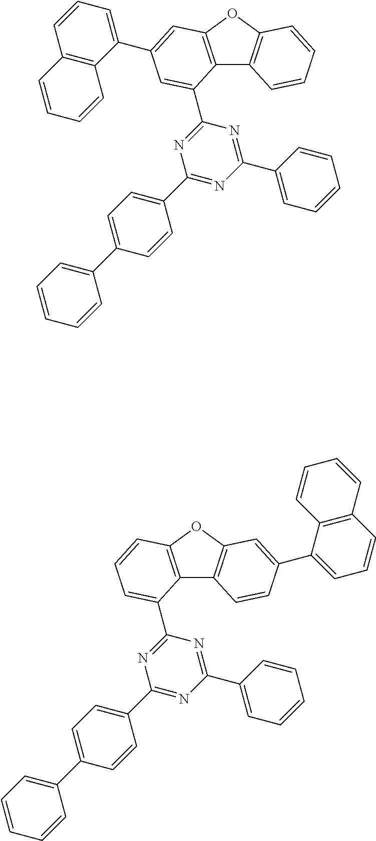 [SMILES: c1ccc(-c2ccc(-c3nc(-c4ccccc4)nc(-c4cc(-c5cccc6ccccc56)cc5oc6ccccc6c45)n3)cc2)cc1 ; c1ccc(-c2ccc(-c3nc(-c4ccccc4)nc(-c4cccc5oc6cc(-c7cccc8ccccc78)ccc6c45)n3)cc2)cc1]

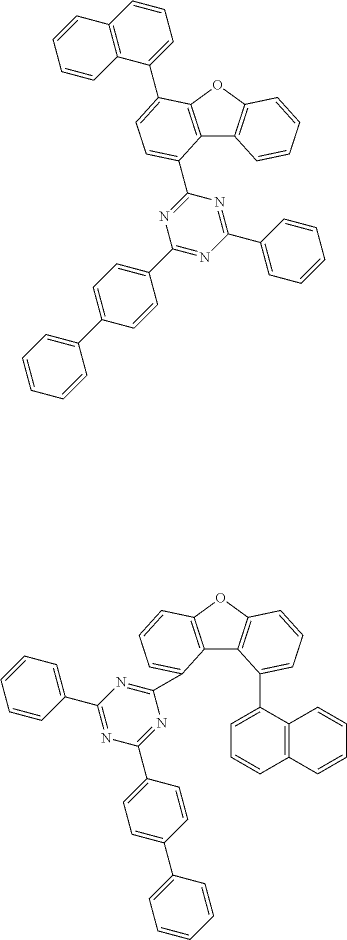 [SMILES: c1ccc(-c2ccc(-c3nc(-c4ccccc4)nc(-c4ccc(-c5cccc6ccccc56)c5oc6ccccc6c45)n3)cc2)cc1 ; c1ccc(-c2ccc(-c3nc(-c4ccccc4)nc(-c4cccc5oc6cccc(-c7cccc8ccccc78)c6c45)n3)cc2)cc1]

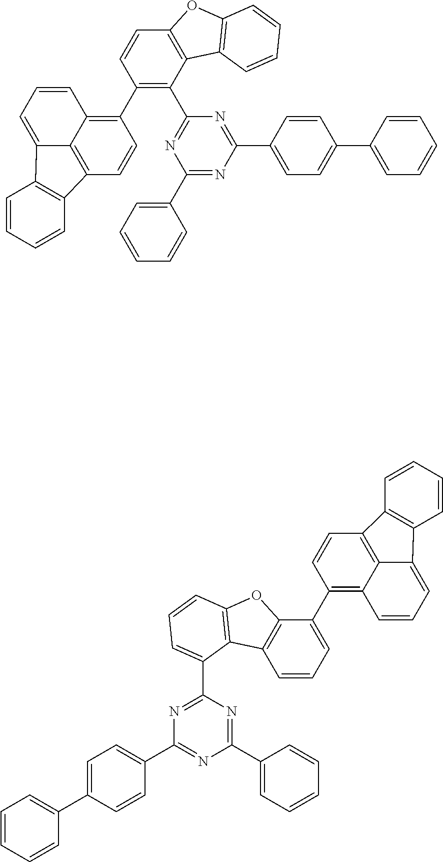 [SMILES: c1ccc(-c2ccc(-c3nc(-c4ccccc4)nc(-c4c(-c5ccc6c7c(cccc57)-c5ccccc5-6)ccc5oc6ccccc6c45)n3)cc2)cc1 ; c1ccc(-c2ccc(-c3nc(-c4ccccc4)nc(-c4cccc5oc6c(-c7ccc8c9c(cccc79)-c7ccccc7-8)cccc6c45)n3)cc2)cc1]

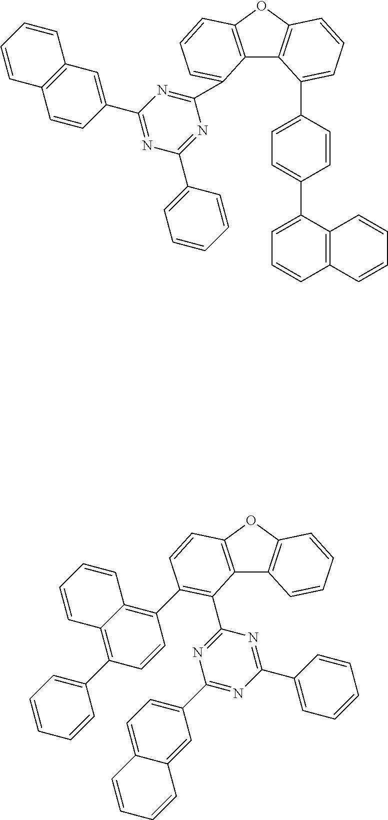 [SMILES: c1ccc(-c2nc(-c3ccc4ccccc4c3)nc(-c3cccc4oc5cccc(-c6ccc(-c7cccc8ccccc78)cc6)c5c34)n2)cc1 ; c1ccc(-c2nc(-c3ccc4ccccc4c3)nc(-c3c(-c4ccc(-c5ccccc5)c5ccccc45)ccc4oc5ccccc5c34)n2)cc1]

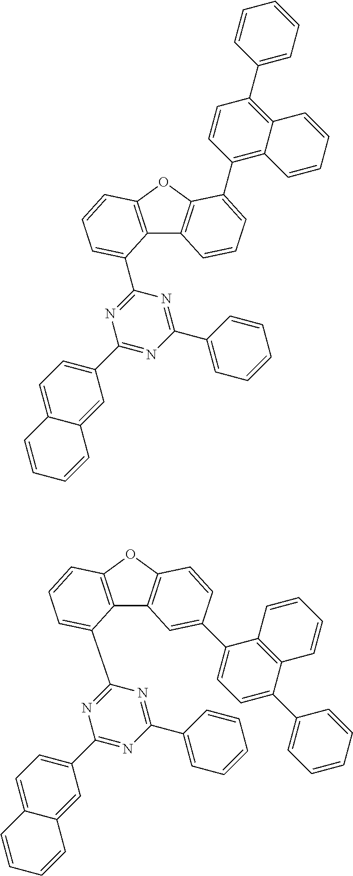 [SMILES: c1ccc(-c2nc(-c3ccc4ccccc4c3)nc(-c3cccc4oc5c(-c6ccc(-c7ccccc7)c7ccccc67)cccc5c34)n2)cc1 ; c1ccc(-c2nc(-c3ccc4ccccc4c3)nc(-c3cccc4oc5ccc(-c6ccc(-c7ccccc7)c7ccccc67)cc5c34)n2)cc1]

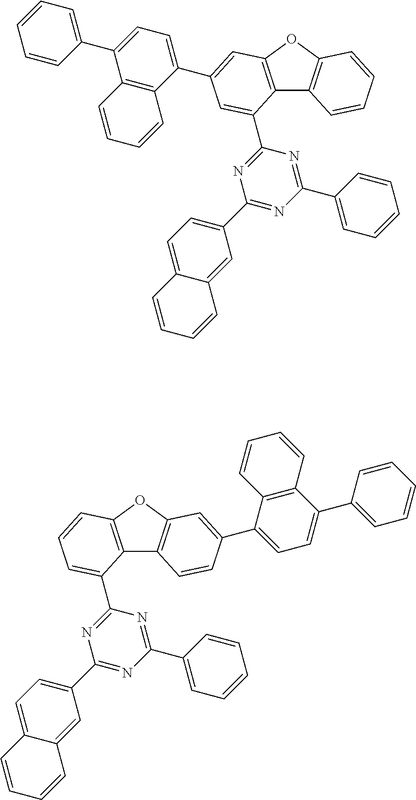 [SMILES: c1ccc(-c2nc(-c3ccc4ccccc4c3)nc(-c3cc(-c4ccc(-c5ccccc5)c5ccccc45)cc4oc5ccccc5c34)n2)cc1 ; c1ccc(-c2nc(-c3ccc4ccccc4c3)nc(-c3cccc4oc5cc(-c6ccc(-c7ccccc7)c7ccccc67)ccc5c34)n2)cc1]

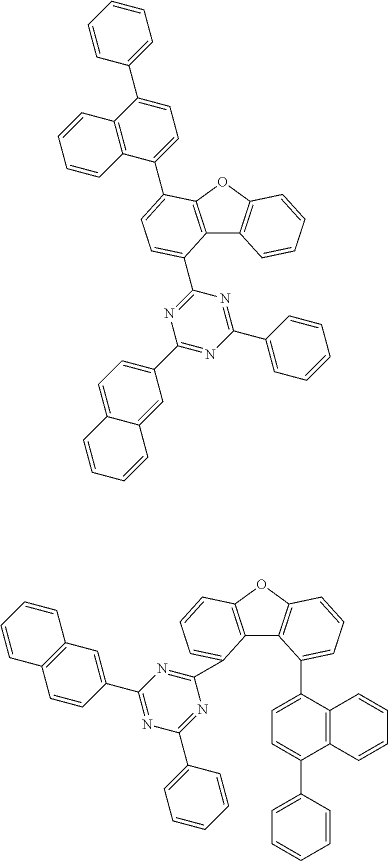 [SMILES: c1ccc(-c2nc(-c3ccc4ccccc4c3)nc(-c3ccc(-c4ccc(-c5ccccc5)c5ccccc45)c4oc5ccccc5c34)n2)cc1 ; c1ccc(-c2nc(-c3ccc4ccccc4c3)nc(-c3cccc4oc5cccc(-c6ccc(-c7ccccc7)c7ccccc67)c5c34)n2)cc1]

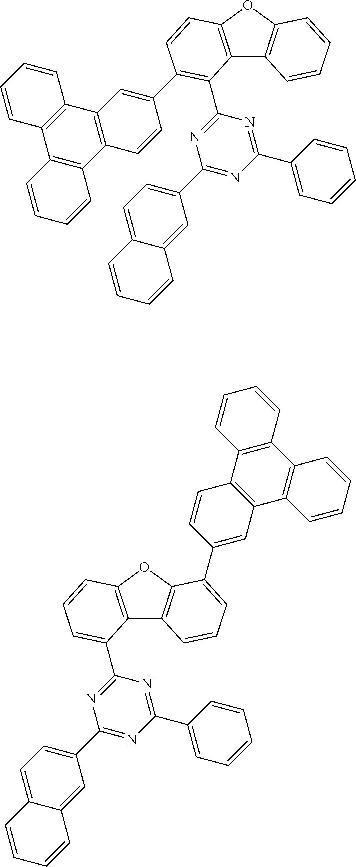 [SMILES: c1ccc(-c2nc(-c3ccc4ccccc4c3)nc(-c3c(-c4ccc5c6ccccc6c6ccccc6c5c4)ccc4oc5ccccc5c34)n2)cc1 ; c1ccc(-c2nc(-c3ccc4ccccc4c3)nc(-c3cccc4oc5c(-c6ccc7c8ccccc8c8ccccc8c7c6)cccc5c34)n2)cc1]

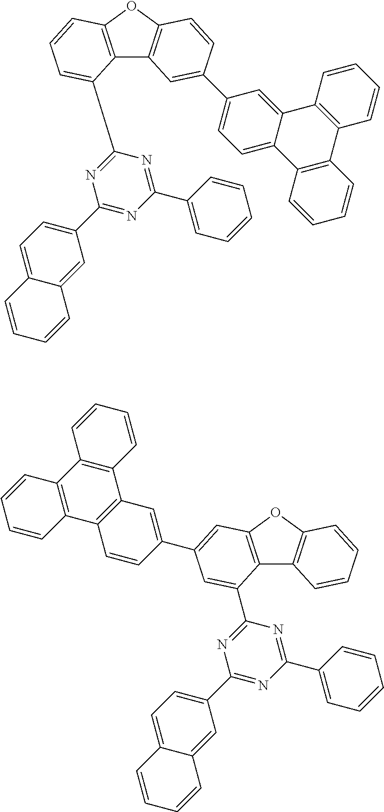 [SMILES: c1ccc(-c2nc(-c3ccc4ccccc4c3)nc(-c3cccc4oc5ccc(-c6ccc7c8ccccc8c8ccccc8c7c6)cc5c34)n2)cc1 ; c1ccc(-c2nc(-c3ccc4ccccc4c3)nc(-c3cc(-c4ccc5c6ccccc6c6ccccc6c5c4)cc4oc5ccccc5c34)n2)cc1]

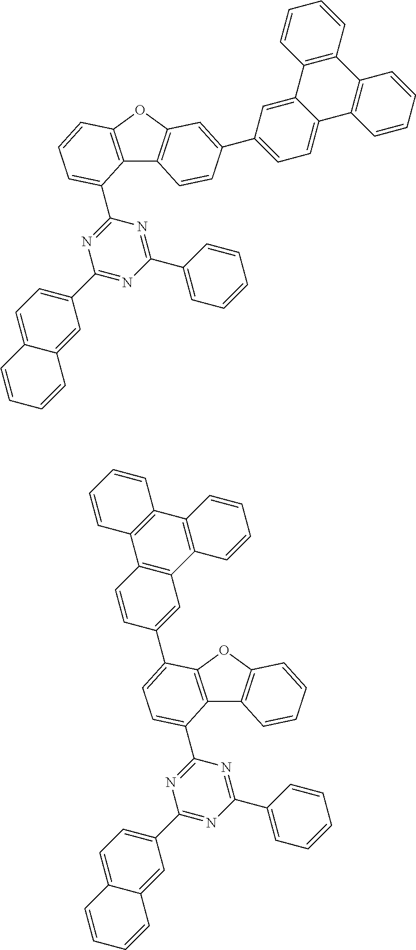 [SMILES: c1ccc(-c2nc(-c3ccc4ccccc4c3)nc(-c3cccc4oc5cc(-c6ccc7c8ccccc8c8ccccc8c7c6)ccc5c34)n2)cc1 ; c1ccc(-c2nc(-c3ccc4ccccc4c3)nc(-c3ccc(-c4ccc5c6ccccc6c6ccccc6c5c4)c4oc5ccccc5c34)n2)cc1]

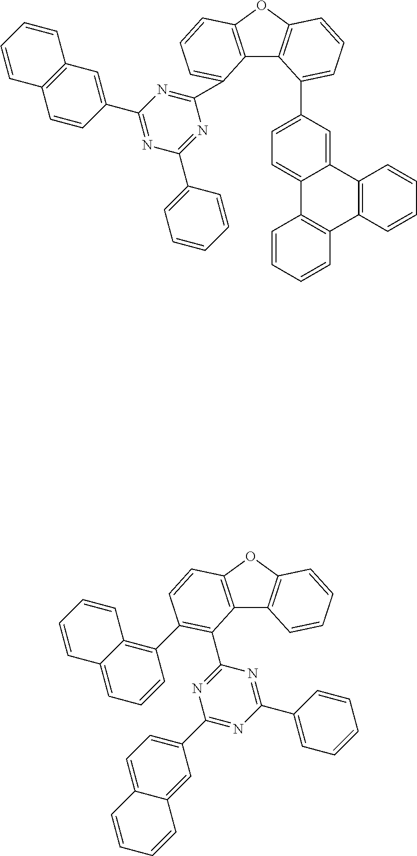 [SMILES: c1ccc(-c2nc(-c3ccc4ccccc4c3)nc(-c3cccc4oc5cccc(-c6ccc7c8ccccc8c8ccccc8c7c6)c5c34)n2)cc1 ; c1ccc(-c2nc(-c3ccc4ccccc4c3)nc(-c3c(-c4cccc5ccccc45)ccc4oc5ccccc5c34)n2)cc1]

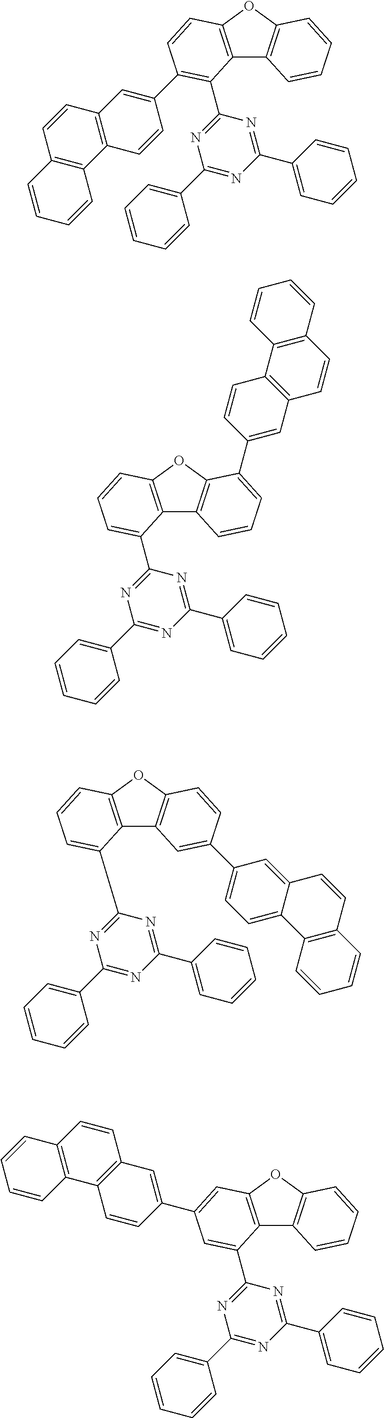 [SMILES: c1ccc(-c2nc(-c3ccccc3)nc(-c3c(-c4ccc5c(ccc6ccccc65)c4)ccc4oc5ccccc5c34)n2)cc1 ; c1ccc(-c2nc(-c3ccccc3)nc(-c3cccc4oc5c(-c6ccc7c(ccc8ccccc87)c6)cccc5c34)n2)cc1 ; c1ccc(-c2nc(-c3ccccc3)nc(-c3cccc4oc5ccc(-c6ccc7c(ccc8ccccc87)c6)cc5c34)n2)cc1 ; c1ccc(-c2nc(-c3ccccc3)nc(-c3cc(-c4ccc5c(ccc6ccccc65)c4)cc4oc5ccccc5c34)n2)cc1]

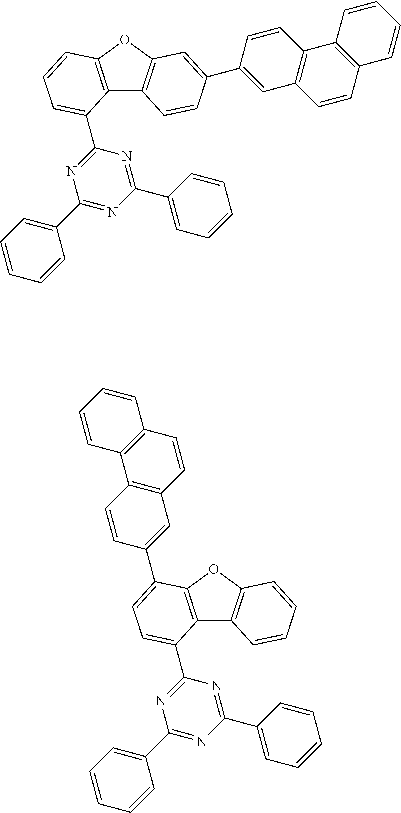 [SMILES: c1ccc(-c2nc(-c3ccccc3)nc(-c3cccc4oc5cc(-c6ccc7c(ccc8ccccc87)c6)ccc5c34)n2)cc1 ; c1ccc(-c2nc(-c3ccccc3)nc(-c3ccc(-c4ccc5c(ccc6ccccc65)c4)c4oc5ccccc5c34)n2)cc1]

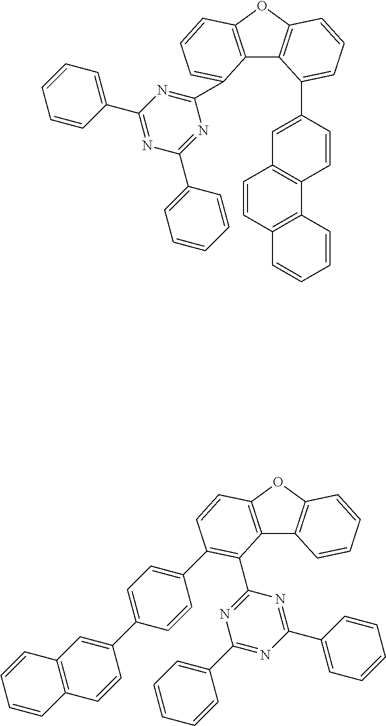 [SMILES: c1ccc(-c2nc(-c3ccccc3)nc(-c3cccc4oc5cccc(-c6ccc7c(ccc8ccccc87)c6)c5c34)n2)cc1 ; c1ccc(-c2nc(-c3ccccc3)nc(-c3c(-c4ccc(-c5ccc6ccccc6c5)cc4)ccc4oc5ccccc5c34)n2)cc1]

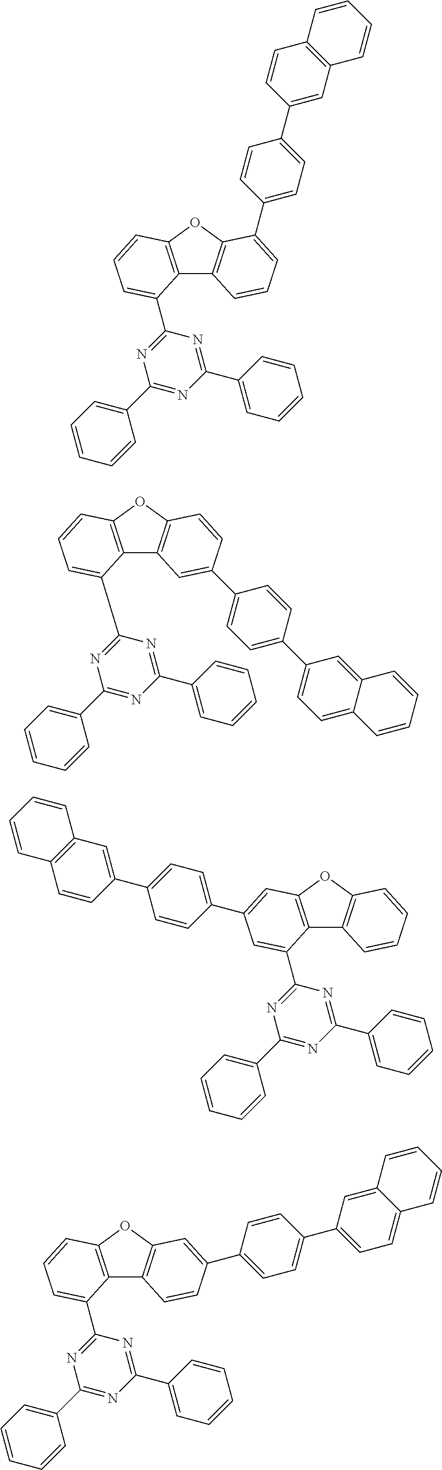

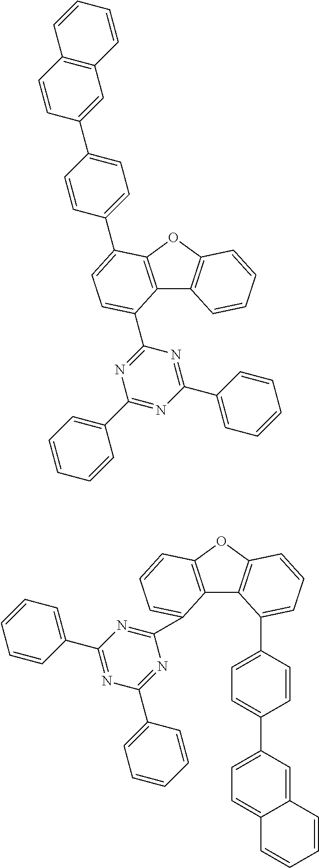 [SMILES: c1ccc(-c2nc(-c3ccccc3)nc(-c3ccc(-c4ccc(-c5ccc6ccccc6c5)cc4)c4oc5ccccc5c34)n2)cc1 ; c1ccc(-c2nc(-c3ccccc3)nc(-c3cccc4oc5cccc(-c6ccc(-c7ccc8ccccc8c7)cc6)c5c34)n2)cc1]

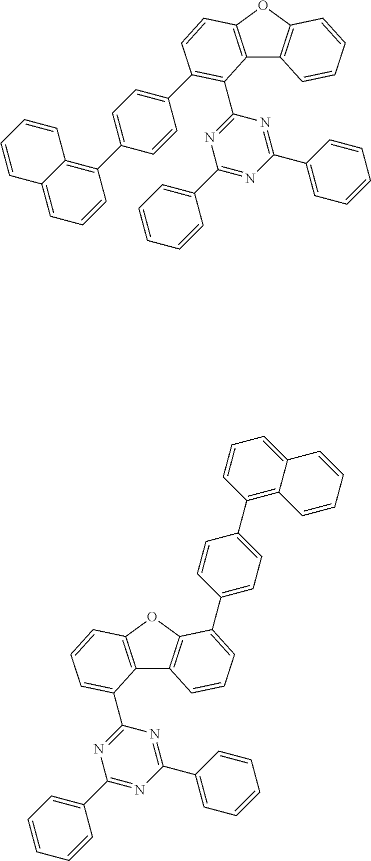 [SMILES: c1ccc(-c2nc(-c3ccccc3)nc(-c3c(-c4ccc(-c5cccc6ccccc56)cc4)ccc4oc5ccccc5c34)n2)cc1 ; c1ccc(-c2nc(-c3ccccc3)nc(-c3cccc4oc5c(-c6ccc(-c7cccc8ccccc78)cc6)cccc5c34)n2)cc1]

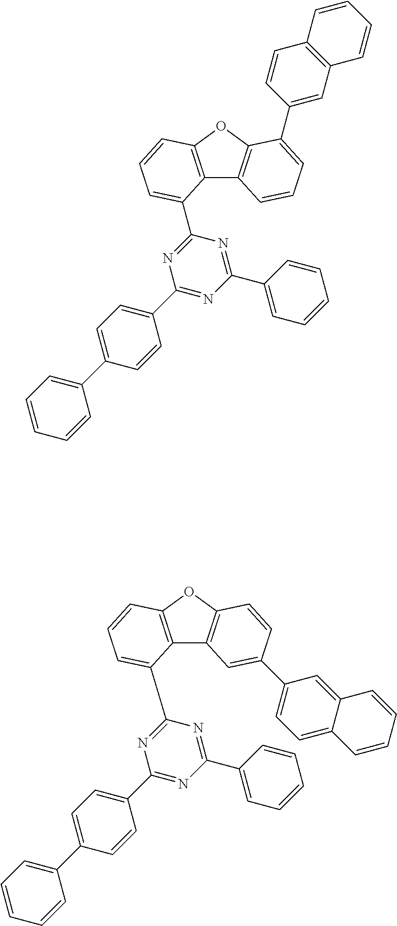 [SMILES: c1ccc(-c2ccc(-c3nc(-c4ccccc4)nc(-c4cccc5oc6c(-c7ccc8ccccc8c7)cccc6c45)n3)cc2)cc1 ; c1ccc(-c2ccc(-c3nc(-c4ccccc4)nc(-c4cccc5oc6ccc(-c7ccc8ccccc8c7)cc6c45)n3)cc2)cc1]

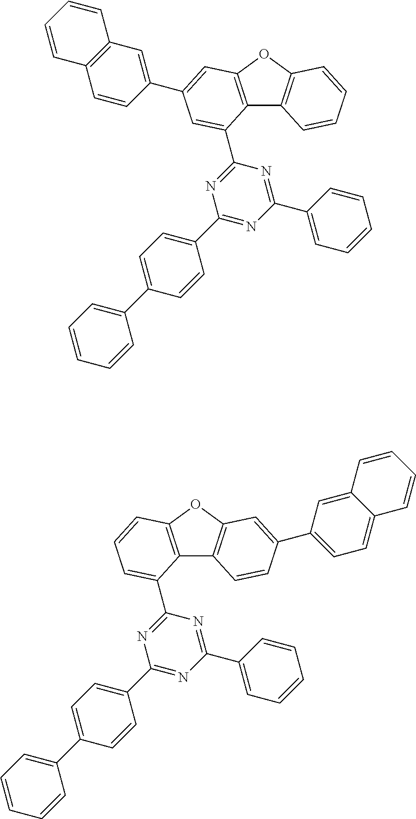 [SMILES: c1ccc(-c2ccc(-c3nc(-c4ccccc4)nc(-c4cc(-c5ccc6ccccc6c5)cc5oc6ccccc6c45)n3)cc2)cc1 ; c1ccc(-c2ccc(-c3nc(-c4ccccc4)nc(-c4cccc5oc6cc(-c7ccc8ccccc8c7)ccc6c45)n3)cc2)cc1]

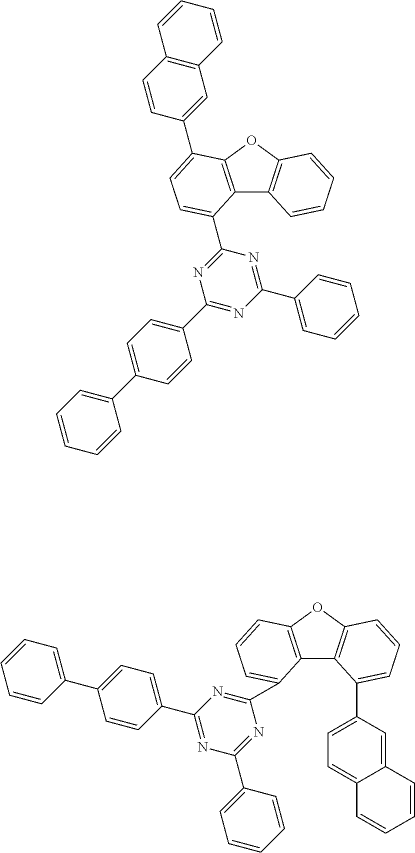 [SMILES: c1ccc(-c2ccc(-c3nc(-c4ccccc4)nc(-c4ccc(-c5ccc6ccccc6c5)c5oc6ccccc6c45)n3)cc2)cc1 ; c1ccc(-c2ccc(-c3nc(-c4ccccc4)nc(-c4cccc5oc6cccc(-c7ccc8ccccc8c7)c6c45)n3)cc2)cc1]

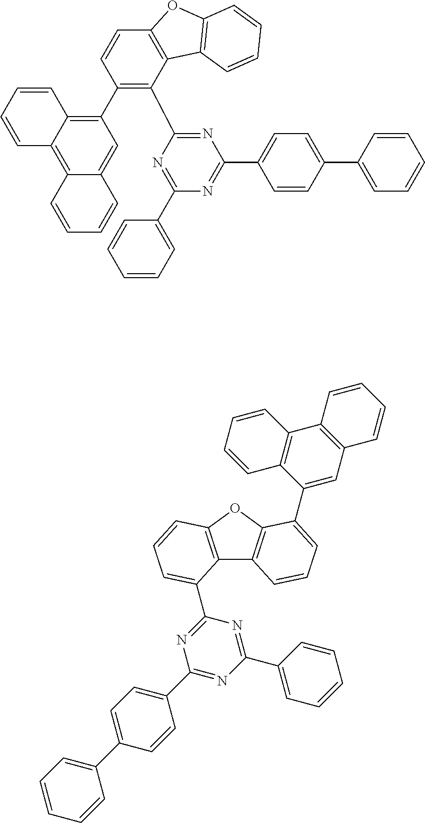 [SMILES: c1ccc(-c2ccc(-c3nc(-c4ccccc4)nc(-c4c(-c5cc6ccccc6c6ccccc56)ccc5oc6ccccc6c45)n3)cc2)cc1 ; c1ccc(-c2ccc(-c3nc(-c4ccccc4)nc(-c4cccc5oc6c(-c7cc8ccccc8c8ccccc78)cccc6c45)n3)cc2)cc1]

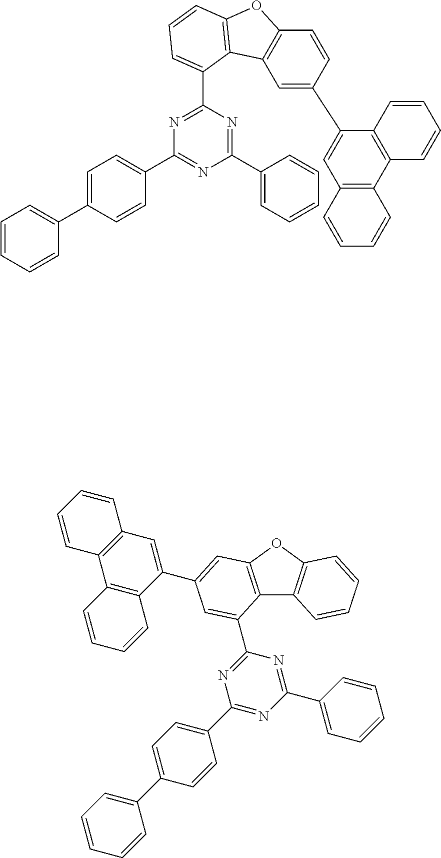 [SMILES: c1ccc(-c2ccc(-c3nc(-c4ccccc4)nc(-c4cccc5oc6ccc(-c7cc8ccccc8c8ccccc78)cc6c45)n3)cc2)cc1 ; c1ccc(-c2ccc(-c3nc(-c4ccccc4)nc(-c4cc(-c5cc6ccccc6c6ccccc56)cc5oc6ccccc6c45)n3)cc2)cc1]

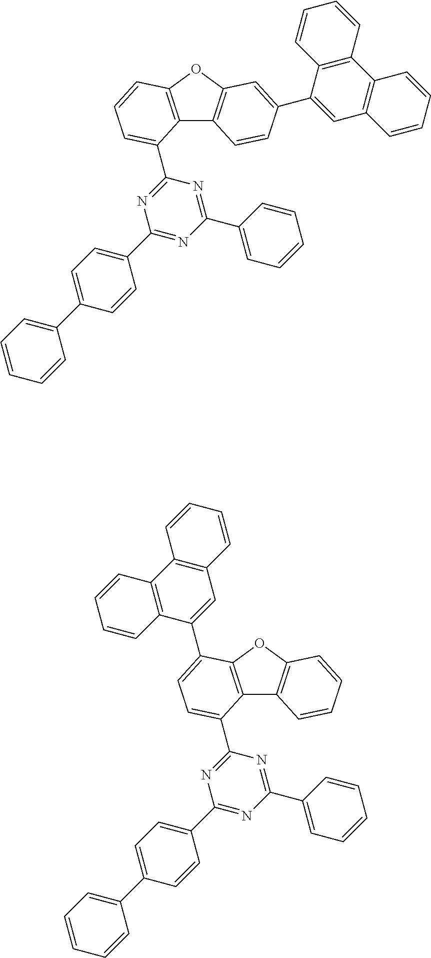 [SMILES: c1ccc(-c2ccc(-c3nc(-c4ccccc4)nc(-c4cccc5oc6cc(-c7cc8ccccc8c8ccccc78)ccc6c45)n3)cc2)cc1 ; c1ccc(-c2ccc(-c3nc(-c4ccccc4)nc(-c4ccc(-c5cc6ccccc6c6ccccc56)c5oc6ccccc6c45)n3)cc2)cc1]

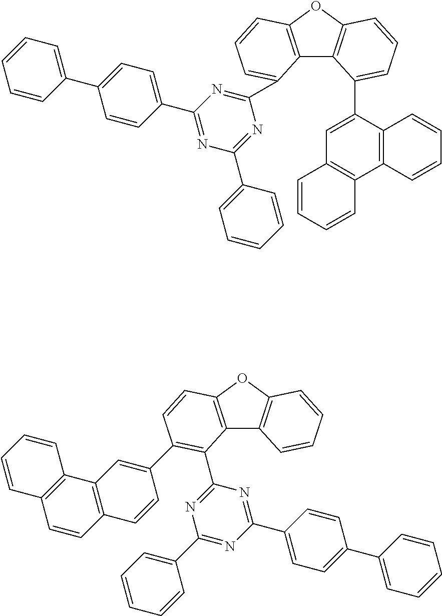 [SMILES: c1ccc(-c2ccc(-c3nc(-c4ccccc4)nc(-c4cccc5oc6cccc(-c7cc8ccccc8c8ccccc78)c6c45)n3)cc2)cc1 ; c1ccc(-c2ccc(-c3nc(-c4ccccc4)nc(-c4c(-c5ccc6ccc7ccccc7c6c5)ccc5oc6ccccc6c45)n3)cc2)cc1]

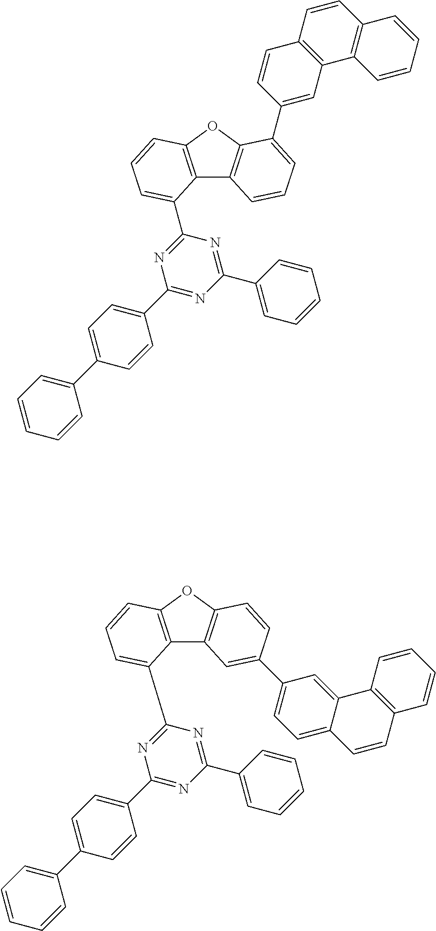 [SMILES: c1ccc(-c2ccc(-c3nc(-c4ccccc4)nc(-c4cccc5oc6c(-c7ccc8ccc9ccccc9c8c7)cccc6c45)n3)cc2)cc1 ; c1ccc(-c2ccc(-c3nc(-c4ccccc4)nc(-c4cccc5oc6ccc(-c7ccc8ccc9ccccc9c8c7)cc6c45)n3)cc2)cc1]

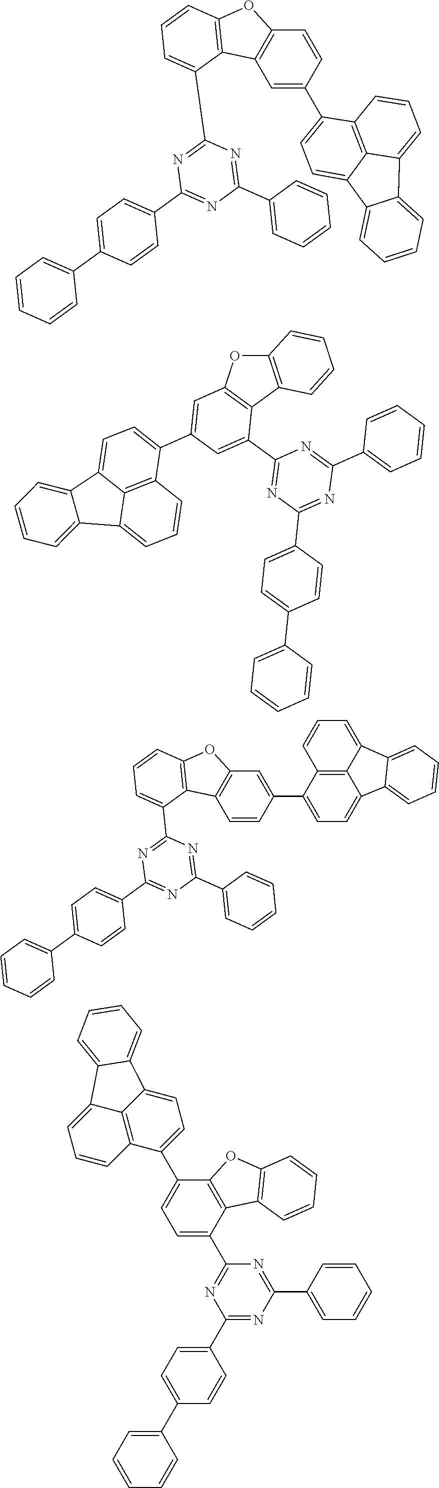

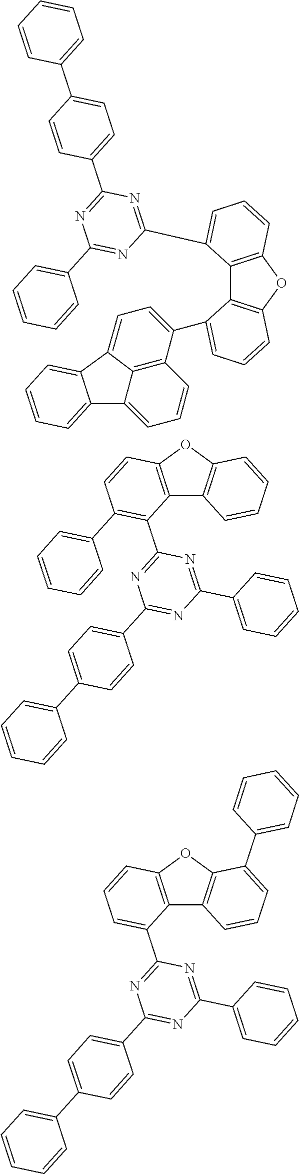

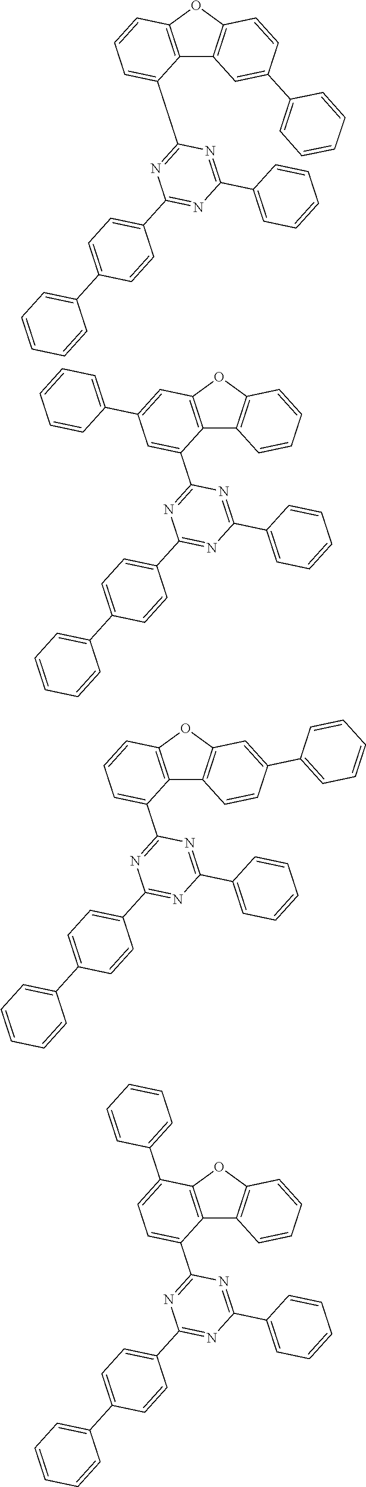

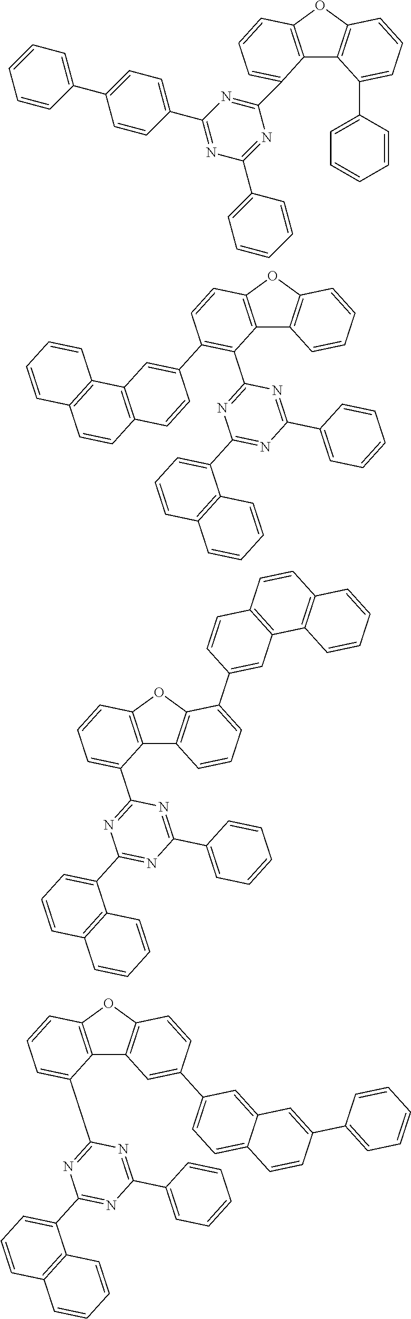

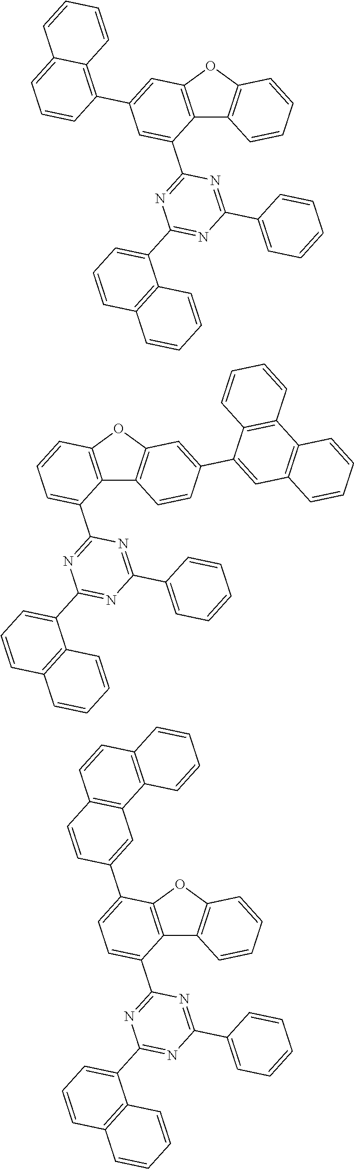

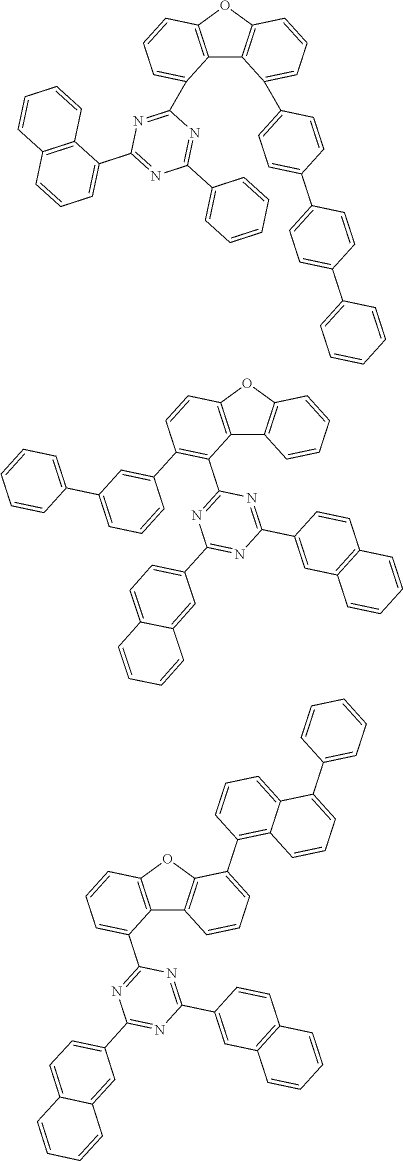

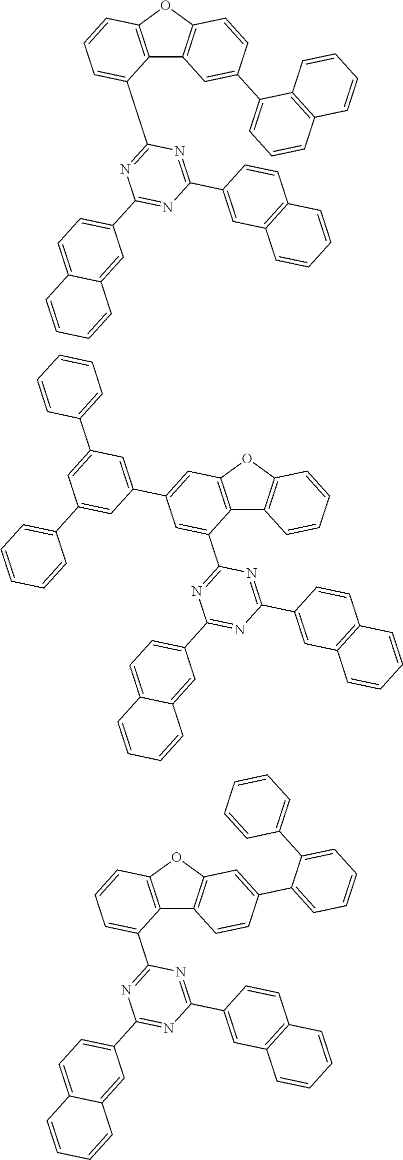

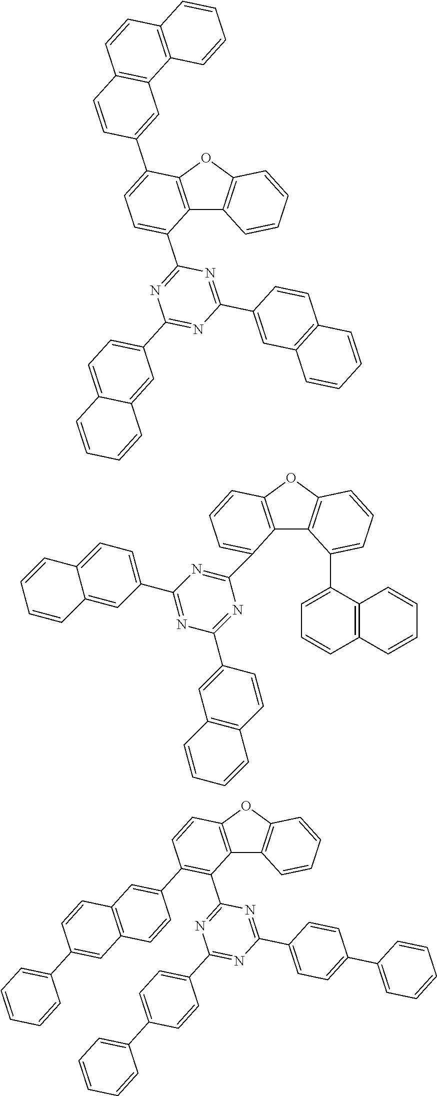

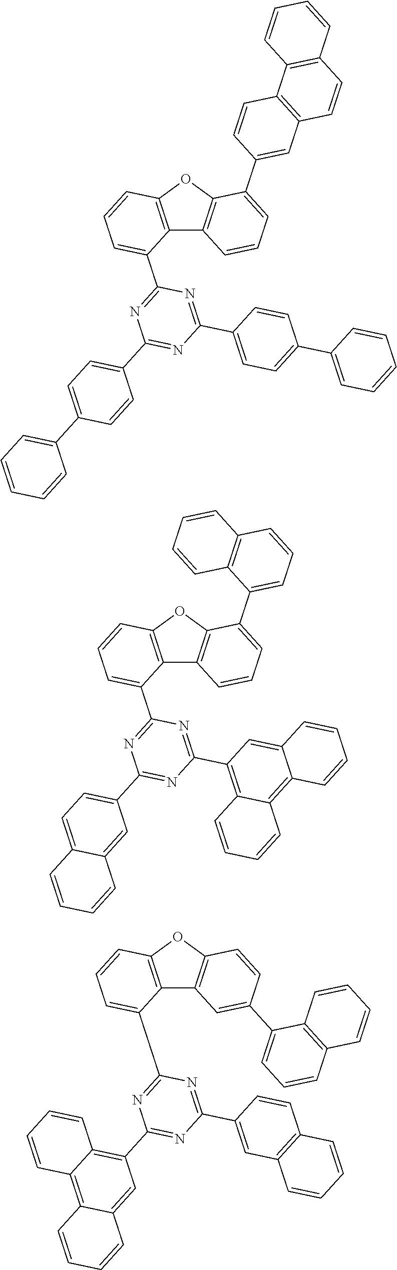

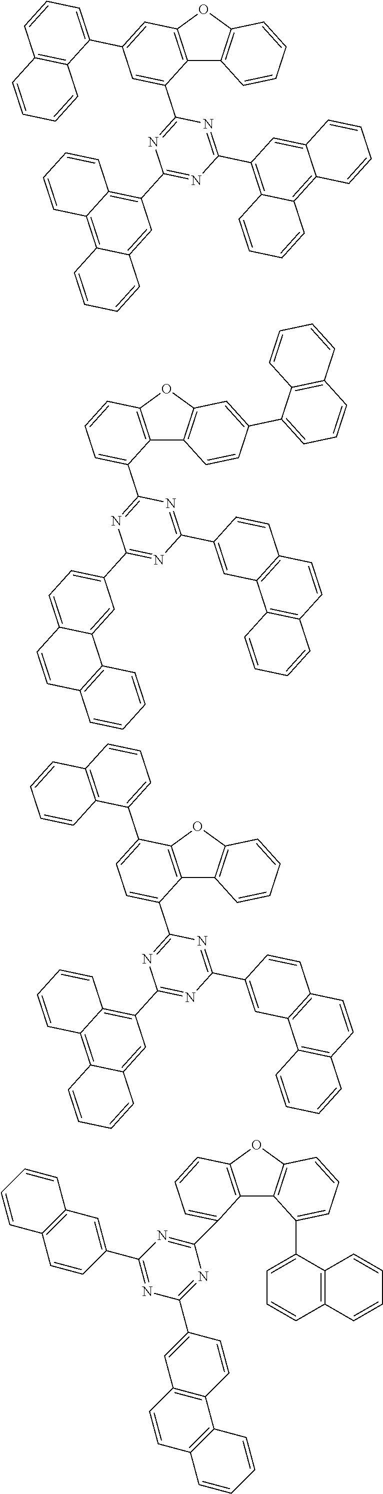

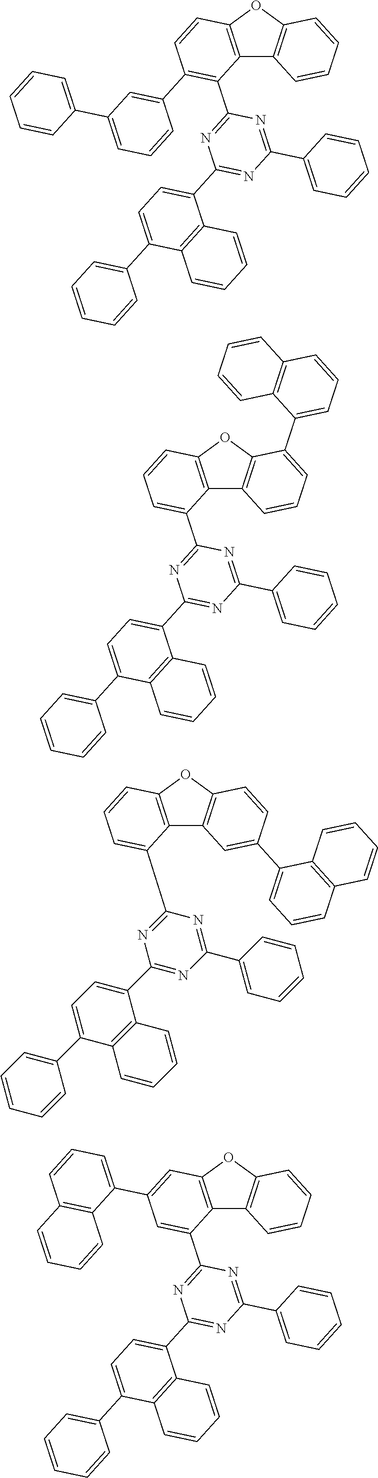

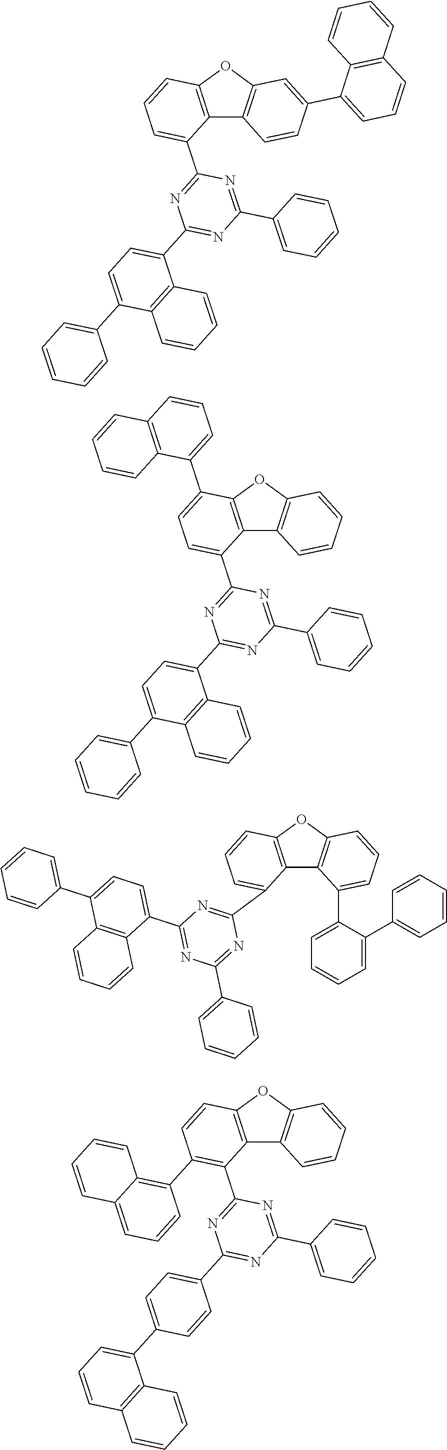

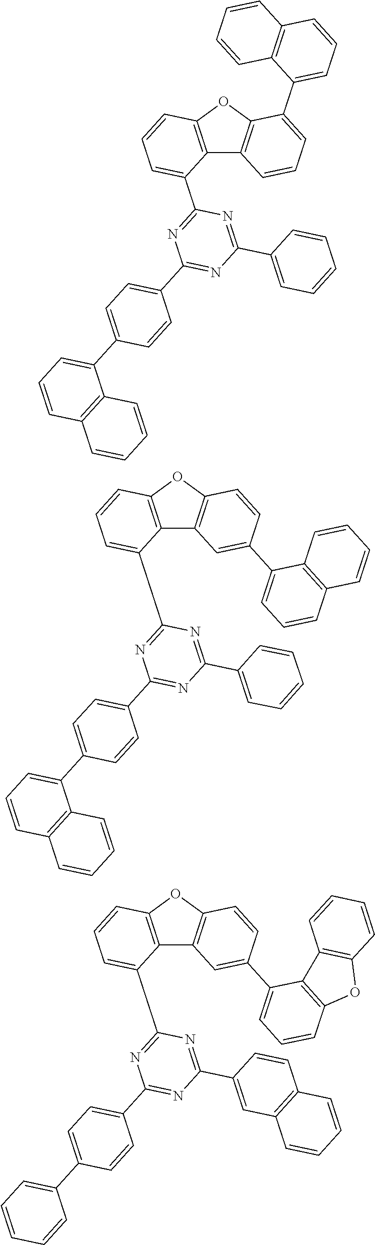

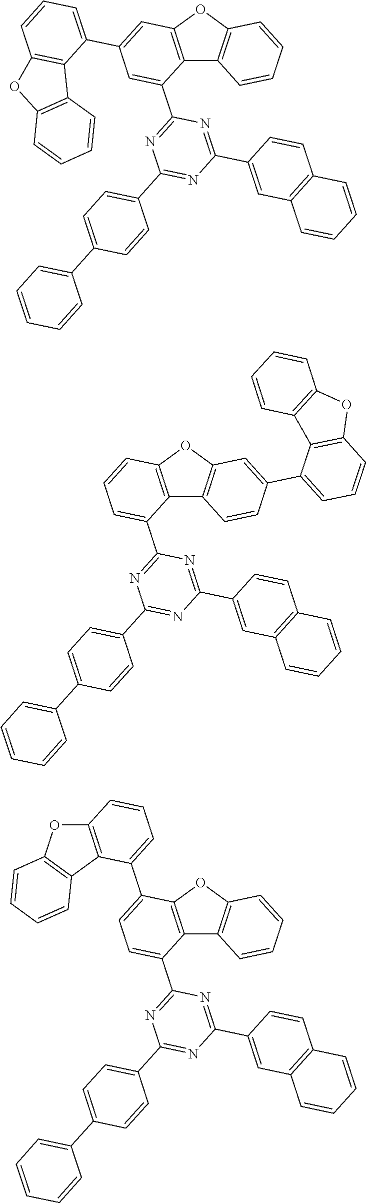 [SMILES: c1ccc(-c2ccc(-c3nc(-c4ccc5ccccc5c4)nc(-c4cc(-c5cccc6oc7ccccc7c56)cc5oc6ccccc6c45)n3)cc2)cc1 ; c1ccc(-c2ccc(-c3nc(-c4ccc5ccccc5c4)nc(-c4cccc5oc6cc(-c7cccc8oc9ccccc9c78)ccc6c45)n3)cc2)cc1 ; c1ccc(-c2ccc(-c3nc(-c4ccc5ccccc5c4)nc(-c4ccc(-c5cccc6oc7ccccc7c56)c5oc6ccccc6c45)n3)cc2)cc1]

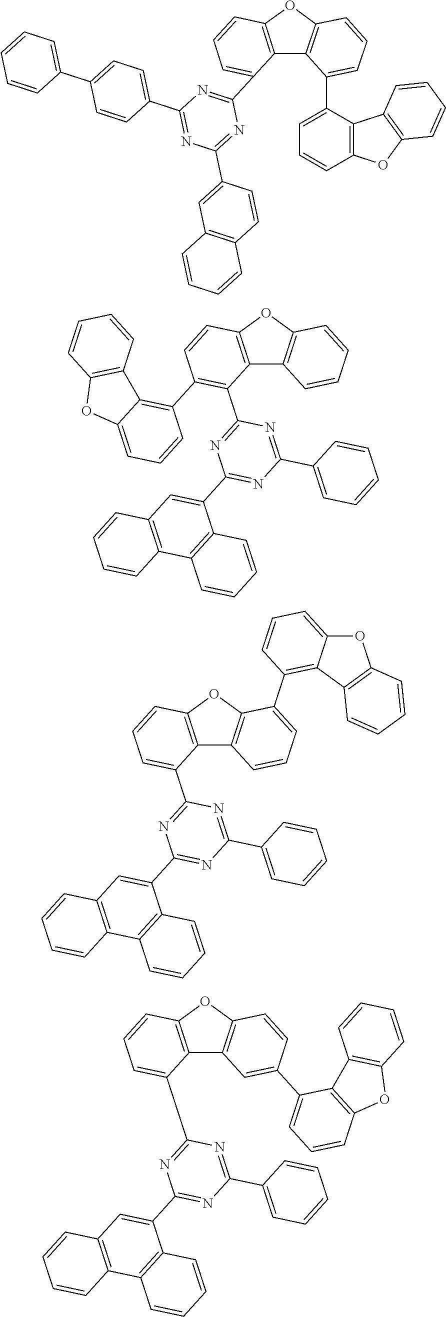

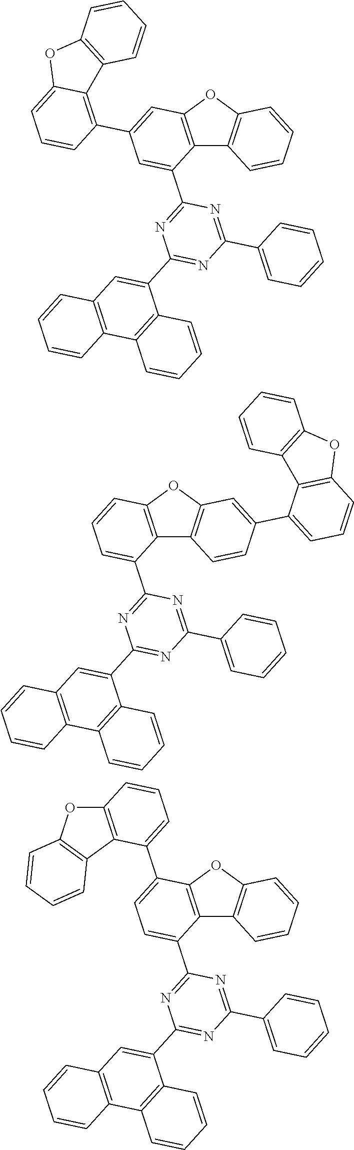

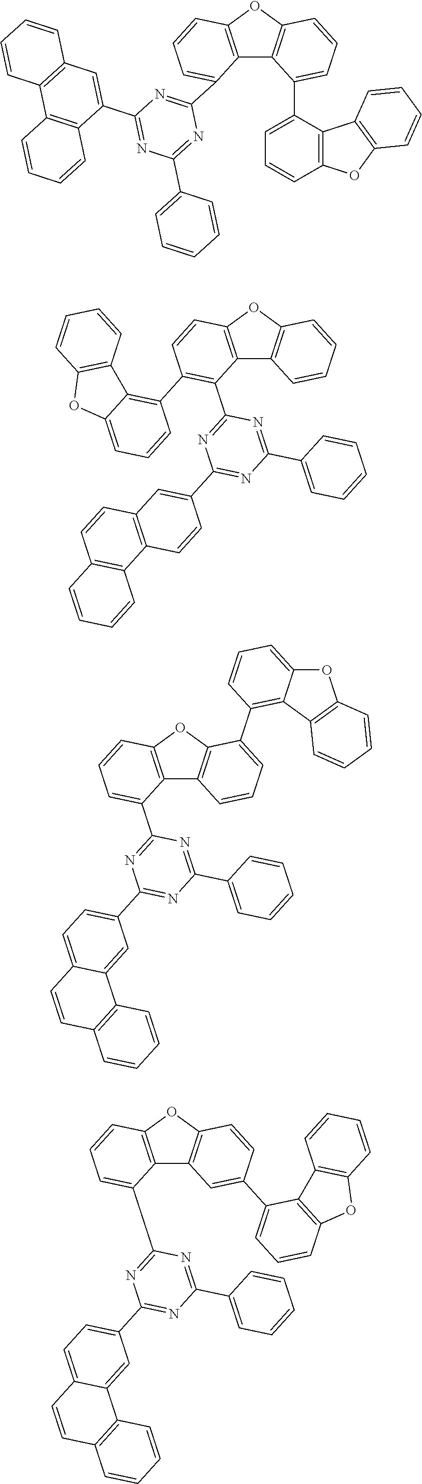 [SMILES: c1ccc(-c2nc(-c3cc4ccccc4c4ccccc34)nc(-c3cccc4oc5cccc(-c6cccc7oc8ccccc8c67)c5c34)n2)cc1 ; c1ccc(-c2nc(-c3ccc4c(ccc5ccccc54)c3)nc(-c3c(-c4cccc5oc6ccccc6c45)ccc4oc5ccccc5c34)n2)cc1 ; c1ccc(-c2nc(-c3ccc4ccc5ccccc5c4c3)nc(-c3cccc4oc5c(-c6cccc7oc8ccccc8c67)cccc5c34)n2)cc1 ; c1ccc(-c2nc(-c3ccc4ccc5ccccc5c4c3)nc(-c3cccc4oc5ccc(-c6cccc7oc8ccccc8c67)cc5c34)n2)cc1]

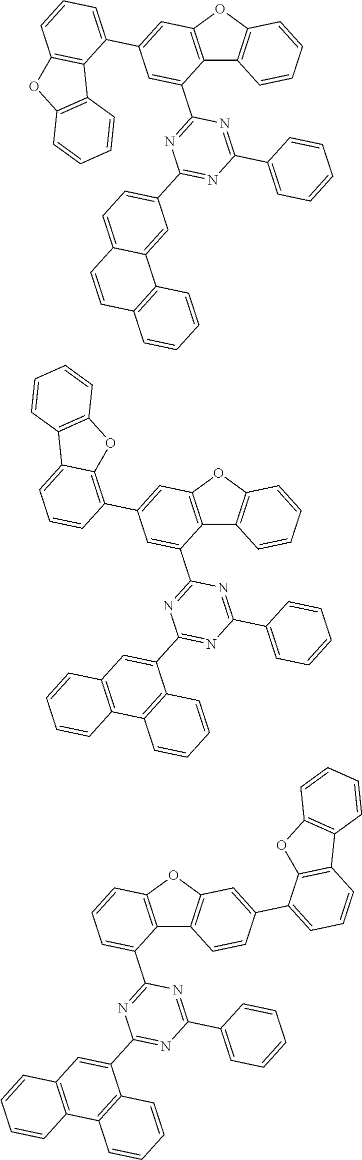

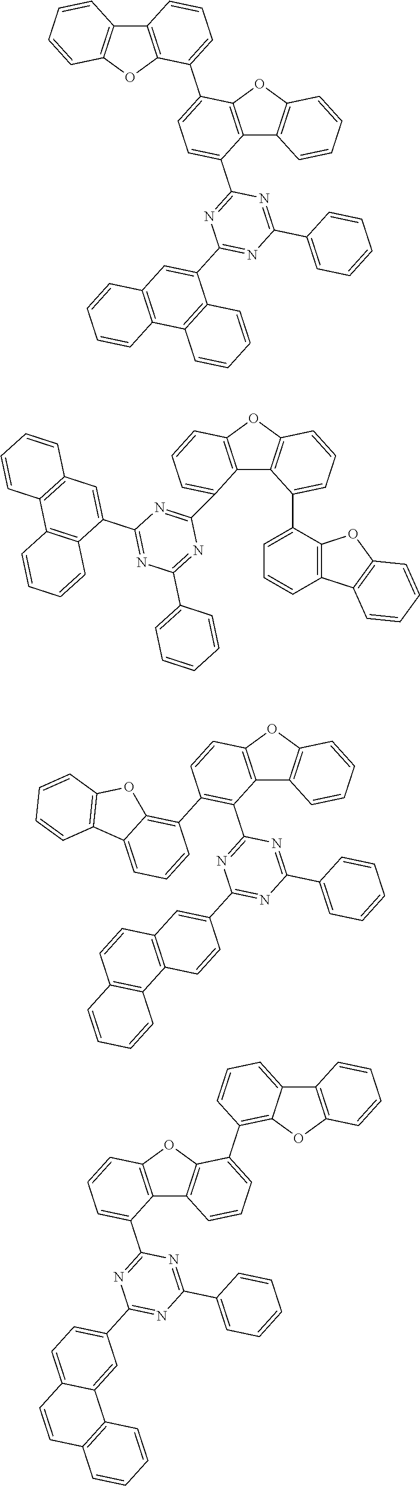 [SMILES: c1ccc(-c2nc(-c3cc4ccccc4c4ccccc34)nc(-c3ccc(-c4cccc5c4oc4ccccc45)c4oc5ccccc5c34)n2)cc1 ; c1ccc(-c2nc(-c3cc4ccccc4c4ccccc34)nc(-c3cccc4oc5cccc(-c6cccc7c6oc6ccccc67)c5c34)n2)cc1 ; c1ccc(-c2nc(-c3ccc4c(ccc5ccccc54)c3)nc(-c3c(-c4cccc5c4oc4ccccc45)ccc4oc5ccccc5c34)n2)cc1 ; c1ccc(-c2nc(-c3ccc4ccc5ccccc5c4c3)nc(-c3cccc4oc5c(-c6cccc7c6oc6ccccc67)cccc5c34)n2)cc1]

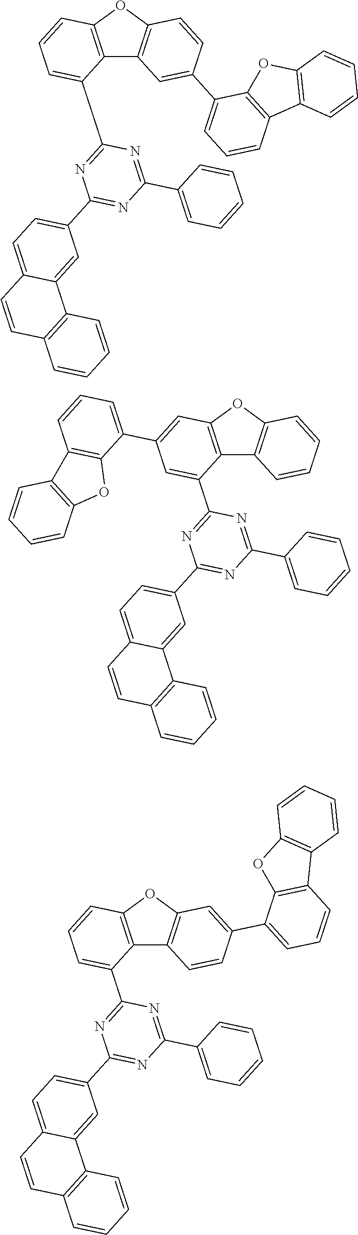

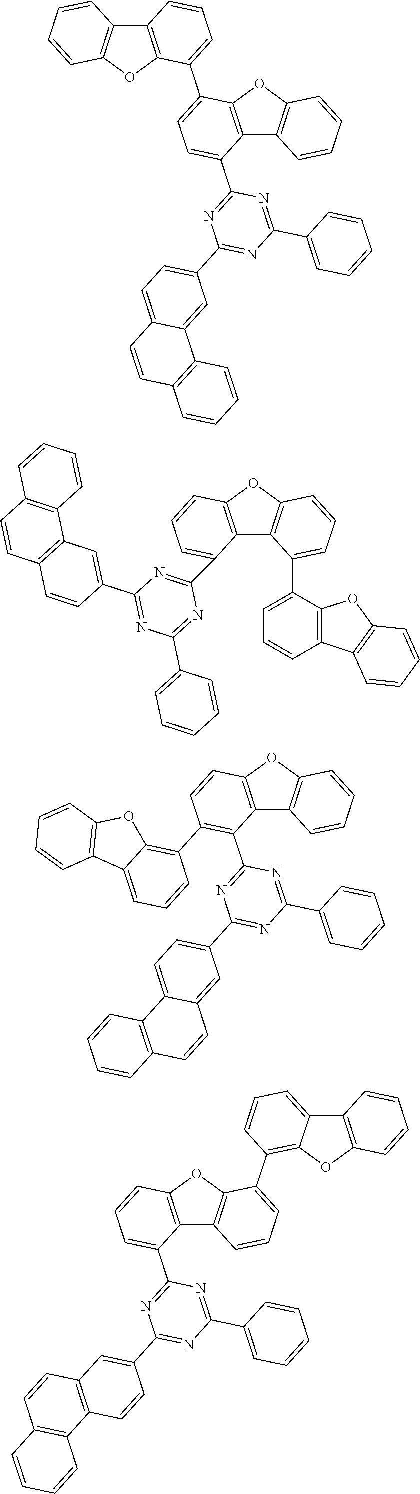

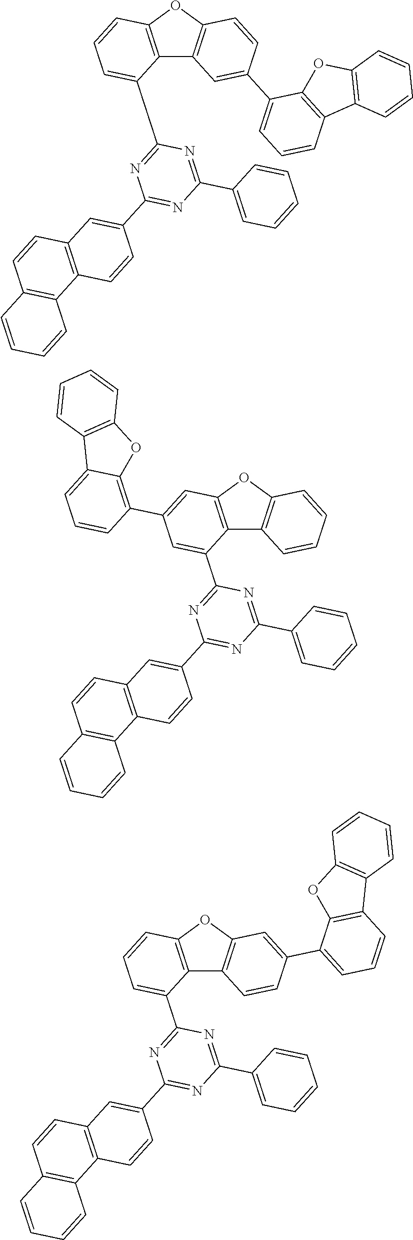

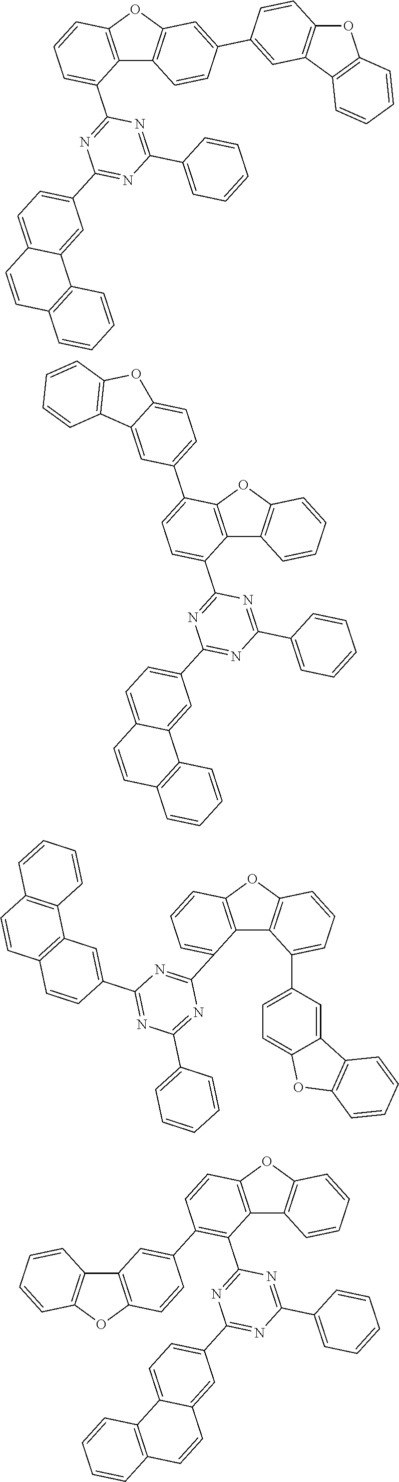

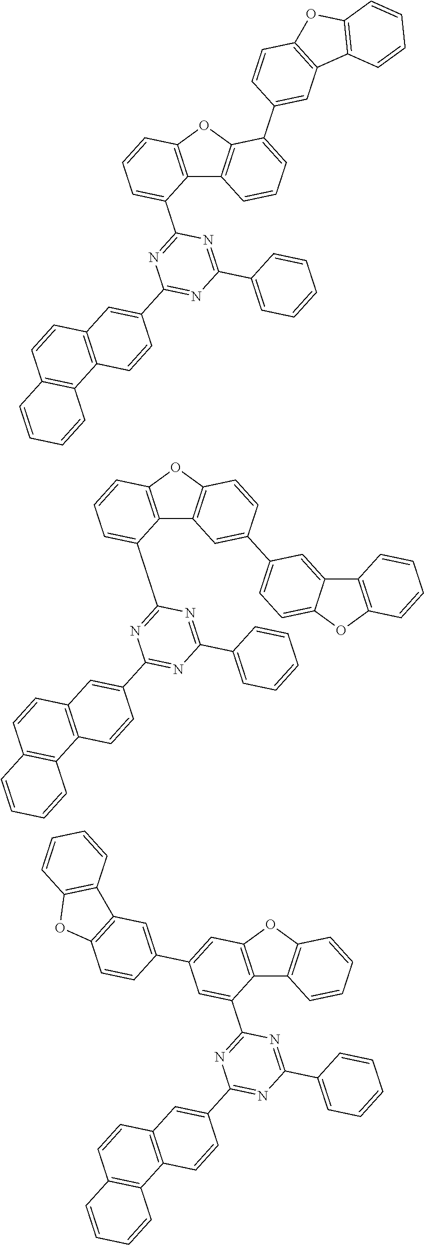

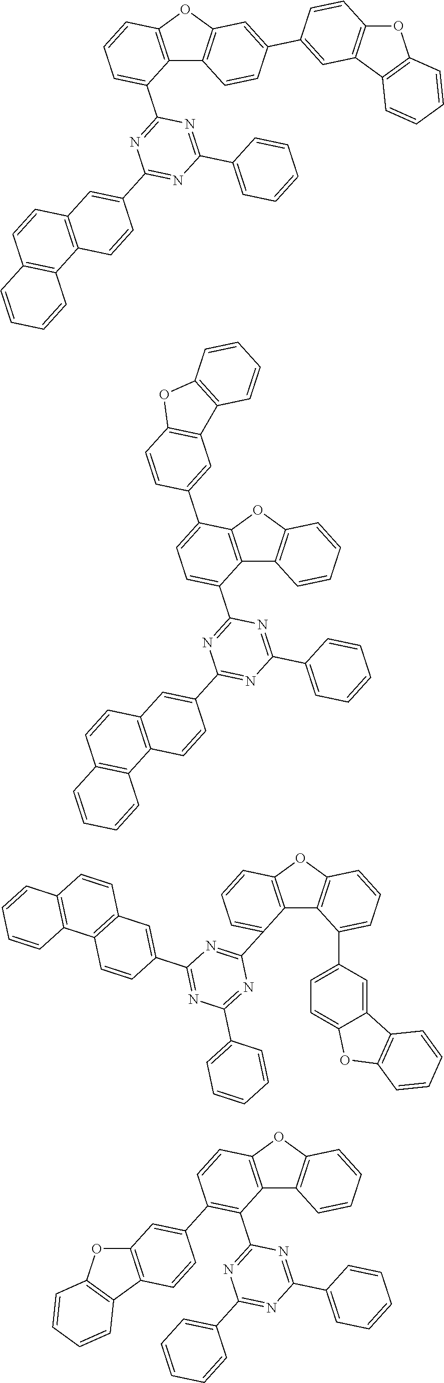 [SMILES: c1ccc(-c2nc(-c3ccc4c(ccc5ccccc54)c3)nc(-c3cccc4oc5cc(-c6ccc7oc8ccccc8c7c6)ccc5c34)n2)cc1 ; c1ccc(-c2nc(-c3ccc4c(ccc5ccccc54)c3)nc(-c3ccc(-c4ccc5oc6ccccc6c5c4)c4oc5ccccc5c34)n2)cc1 ; c1ccc(-c2nc(-c3ccc4c(ccc5ccccc54)c3)nc(-c3cccc4oc5cccc(-c6ccc7oc8ccccc8c7c6)c5c34)n2)cc1 ; c1ccc(-c2nc(-c3ccccc3)nc(-c3c(-c4ccc5c(c4)oc4ccccc45)ccc4oc5ccccc5c34)n2)cc1]

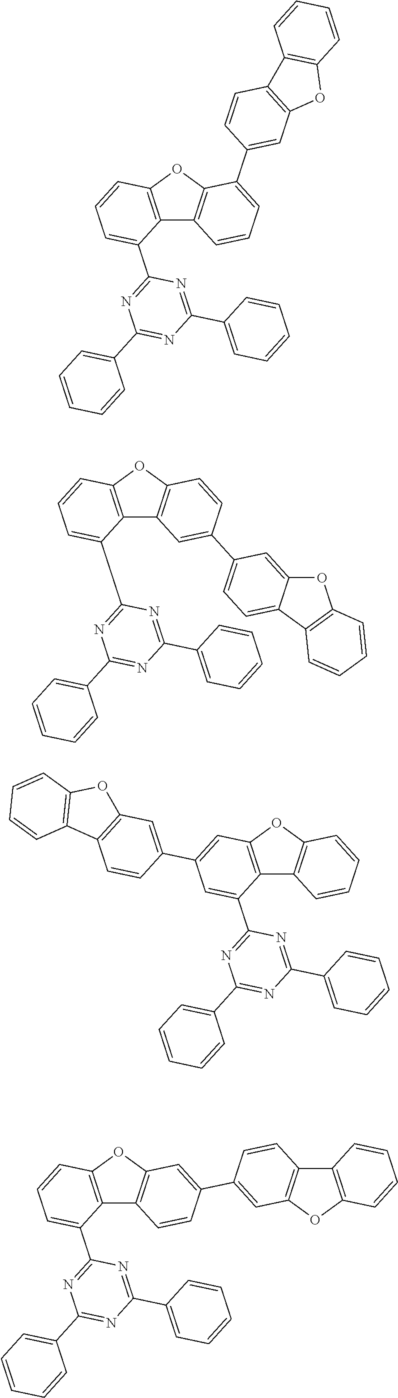

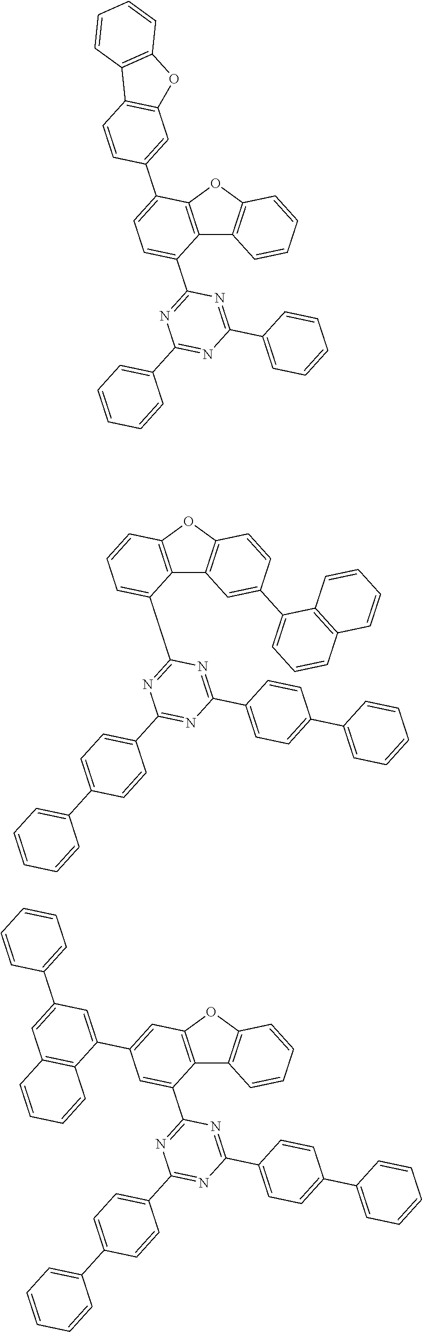

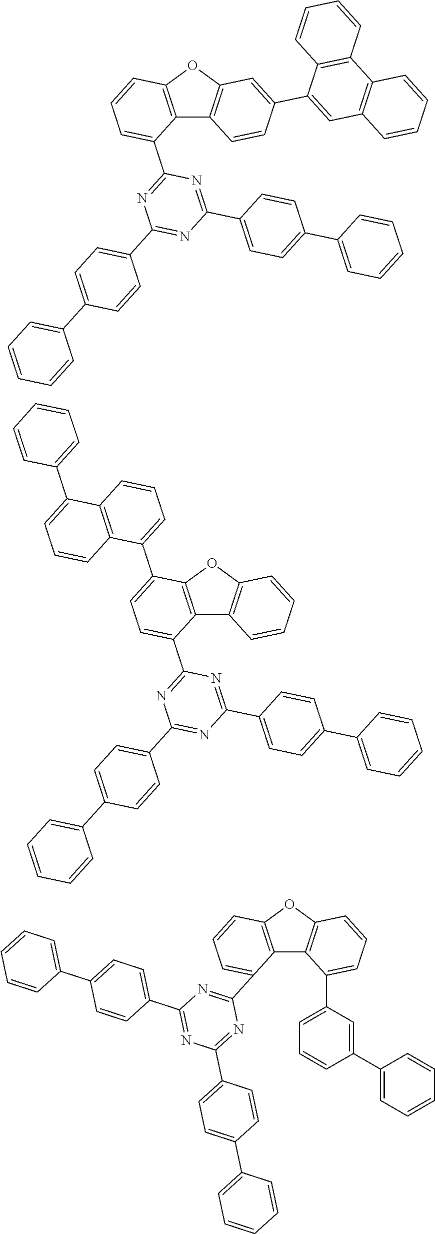

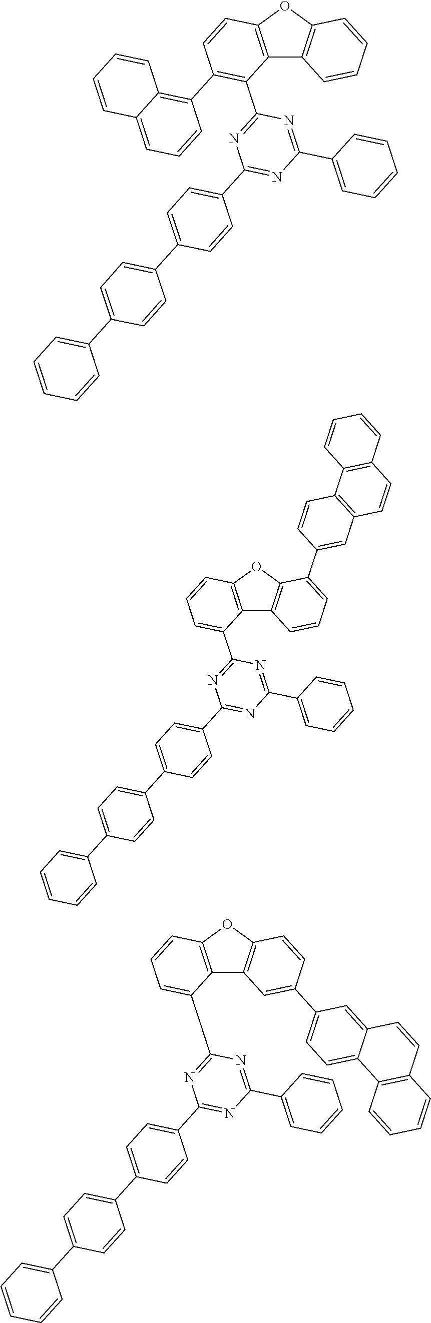 [SMILES: c1ccc(-c2ccc(-c3ccc(-c4nc(-c5ccccc5)nc(-c5c(-c6cccc7ccccc67)ccc6oc7ccccc7c56)n4)cc3)cc2)cc1 ; c1ccc(-c2ccc(-c3ccc(-c4nc(-c5ccccc5)nc(-c5cccc6oc7c(-c8ccc9c(ccc%10ccccc%109)c8)cccc7c56)n4)cc3)cc2)cc1 ; c1ccc(-c2ccc(-c3ccc(-c4nc(-c5ccccc5)nc(-c5cccc6oc7ccc(-c8ccc9c(ccc%10ccccc%109)c8)cc7c56)n4)cc3)cc2)cc1]

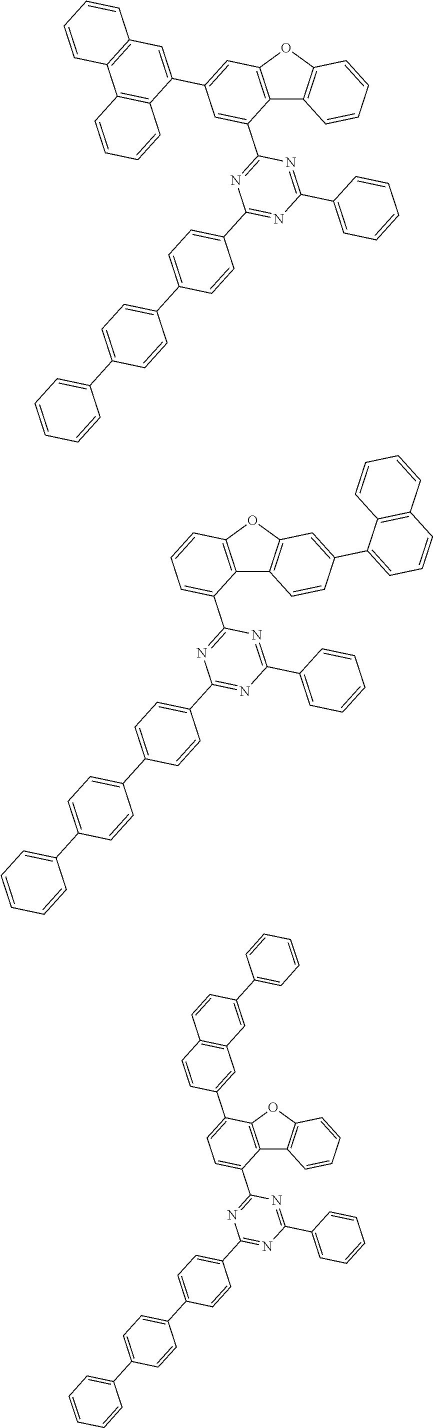 [SMILES: c1ccc(-c2ccc(-c3ccc(-c4nc(-c5ccccc5)nc(-c5cc(-c6cc7ccccc7c7ccccc67)cc6oc7ccccc7c56)n4)cc3)cc2)cc1 ; c1ccc(-c2ccc(-c3ccc(-c4nc(-c5ccccc5)nc(-c5cccc6oc7cc(-c8cccc9ccccc89)ccc7c56)n4)cc3)cc2)cc1 ; c1ccc(-c2ccc(-c3ccc(-c4nc(-c5ccccc5)nc(-c5ccc(-c6ccc7ccc(-c8ccccc8)cc7c6)c6oc7ccccc7c56)n4)cc3)cc2)cc1]

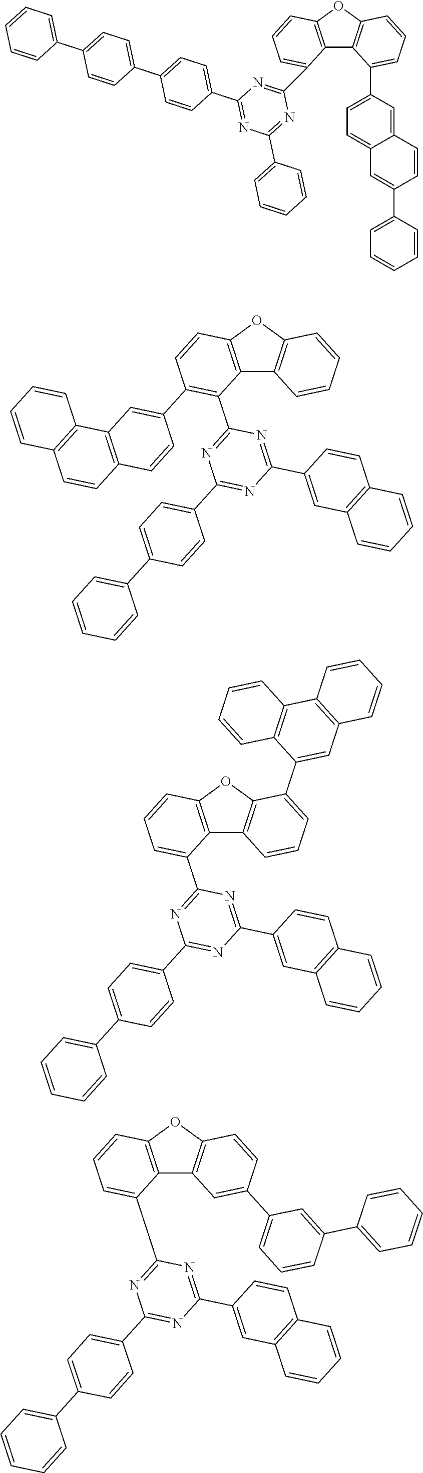

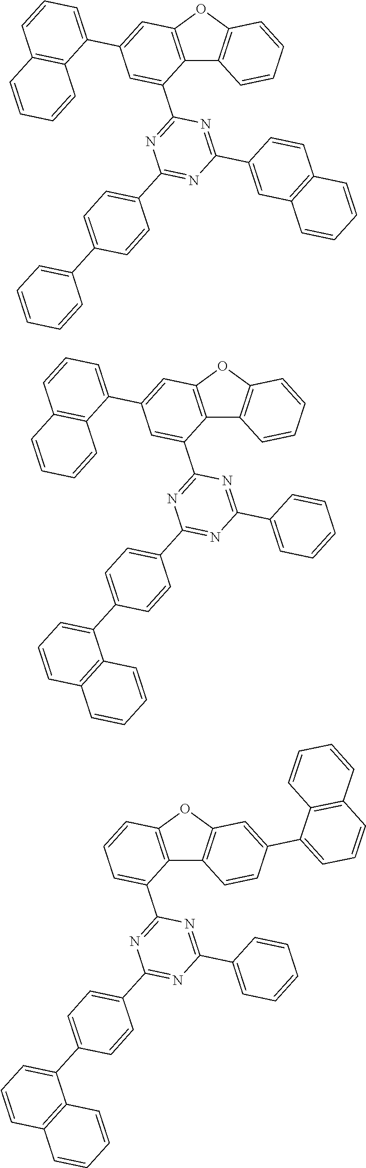

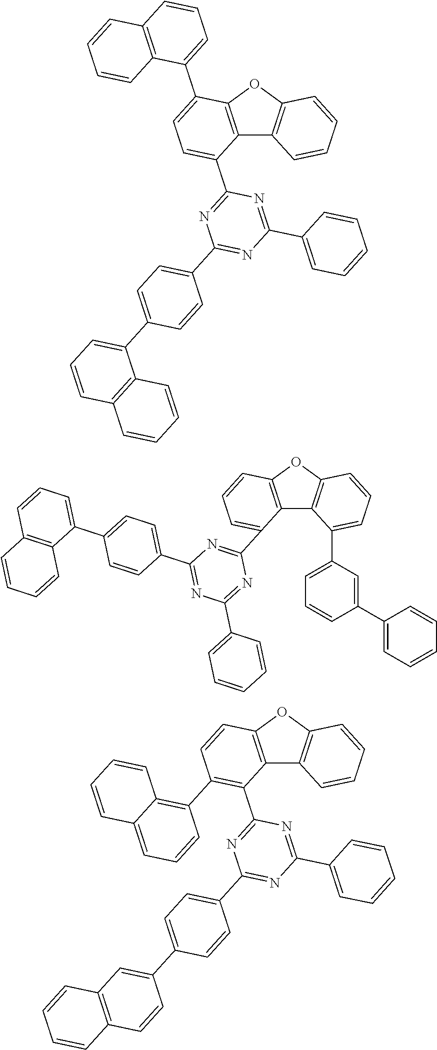

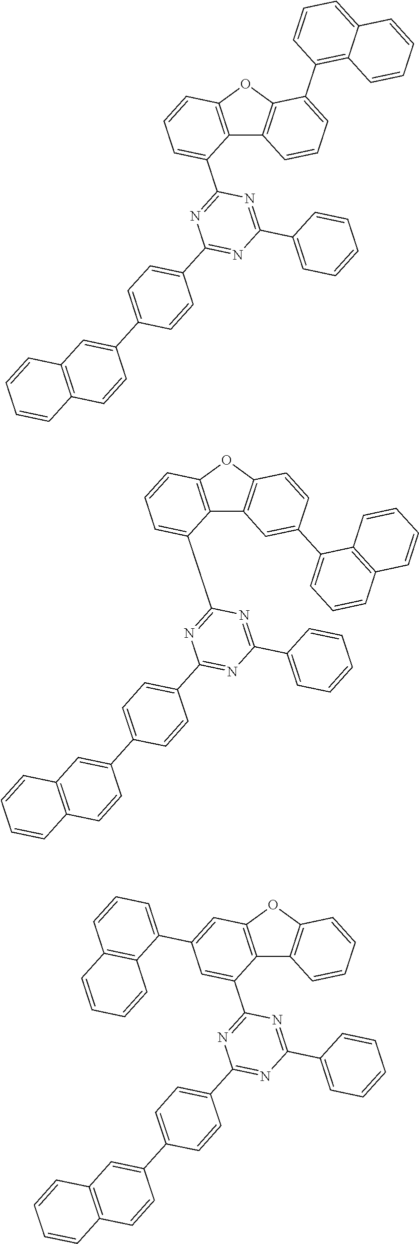 [SMILES: c1ccc(-c2nc(-c3ccc(-c4ccc5ccccc5c4)cc3)nc(-c3cccc4oc5c(-c6cccc7ccccc67)cccc5c34)n2)cc1 ; c1ccc(-c2nc(-c3ccc(-c4ccc5ccccc5c4)cc3)nc(-c3cccc4oc5ccc(-c6cccc7ccccc67)cc5c34)n2)cc1 ; c1ccc(-c2nc(-c3ccc(-c4ccc5ccccc5c4)cc3)nc(-c3cc(-c4cccc5ccccc45)cc4oc5ccccc5c34)n2)cc1]

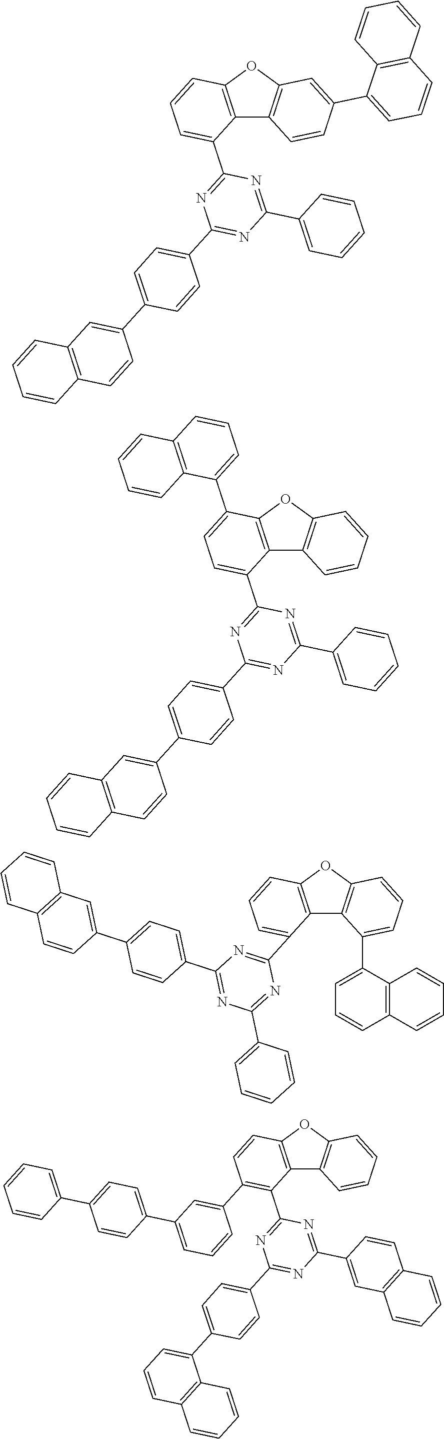

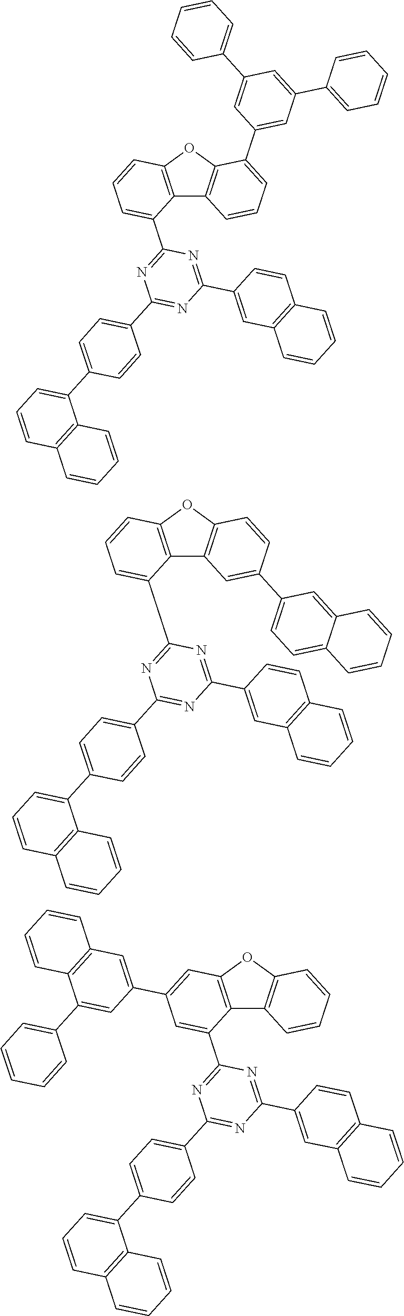

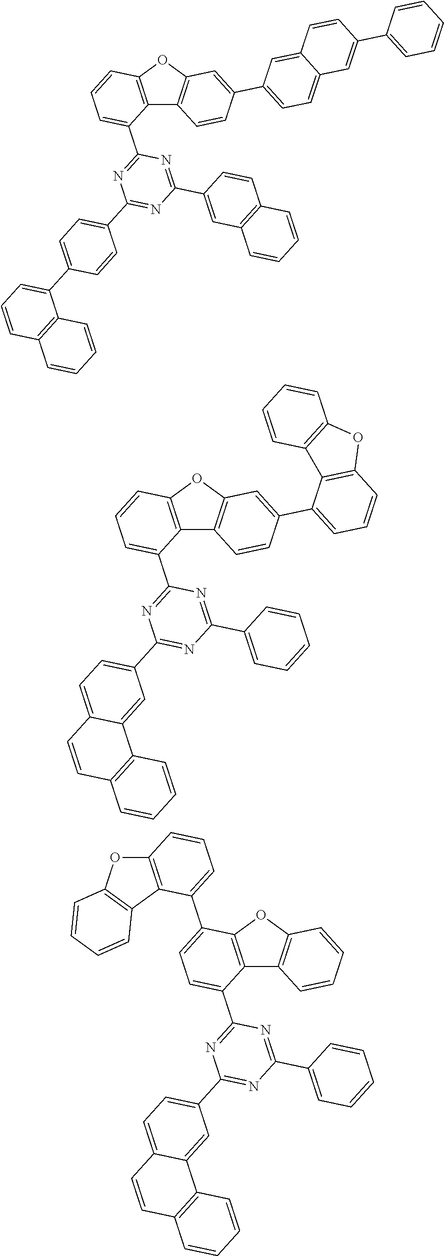

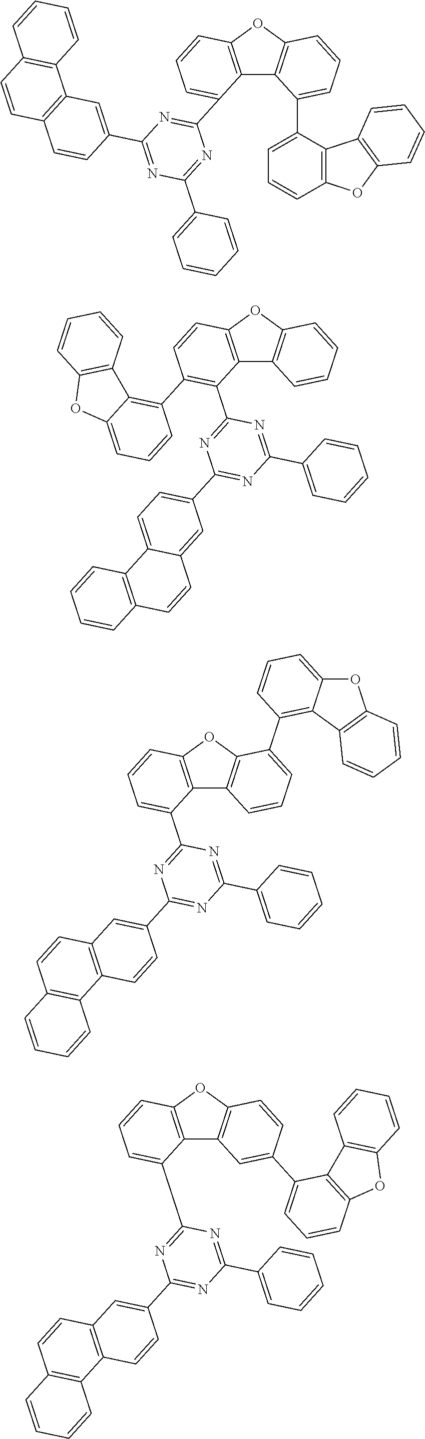 [SMILES: c1ccc(-c2nc(-c3ccc4ccc5ccccc5c4c3)nc(-c3cccc4oc5cccc(-c6cccc7oc8ccccc8c67)c5c34)n2)cc1 ; c1ccc(-c2nc(-c3ccc4c(ccc5ccccc54)c3)nc(-c3c(-c4cccc5oc6ccccc6c45)ccc4oc5ccccc5c34)n2)cc1 ; c1ccc(-c2nc(-c3ccc4c(ccc5ccccc54)c3)nc(-c3cccc4oc5c(-c6cccc7oc8ccccc8c67)cccc5c34)n2)cc1 ; c1ccc(-c2nc(-c3ccc4c(ccc5ccccc54)c3)nc(-c3cccc4oc5ccc(-c6cccc7oc8ccccc8c67)cc5c34)n2)cc1]

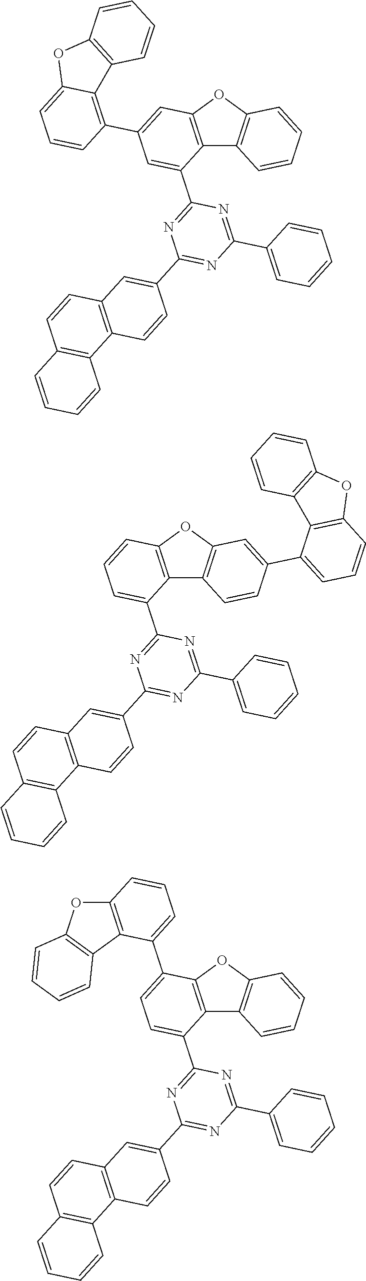 [SMILES: c1ccc(-c2nc(-c3ccc4c(ccc5ccccc54)c3)nc(-c3cc(-c4cccc5oc6ccccc6c45)cc4oc5ccccc5c34)n2)cc1 ; c1ccc(-c2nc(-c3ccc4c(ccc5ccccc54)c3)nc(-c3cccc4oc5cc(-c6cccc7oc8ccccc8c67)ccc5c34)n2)cc1 ; c1ccc(-c2nc(-c3ccc4c(ccc5ccccc54)c3)nc(-c3ccc(-c4cccc5oc6ccccc6c45)c4oc5ccccc5c34)n2)cc1]

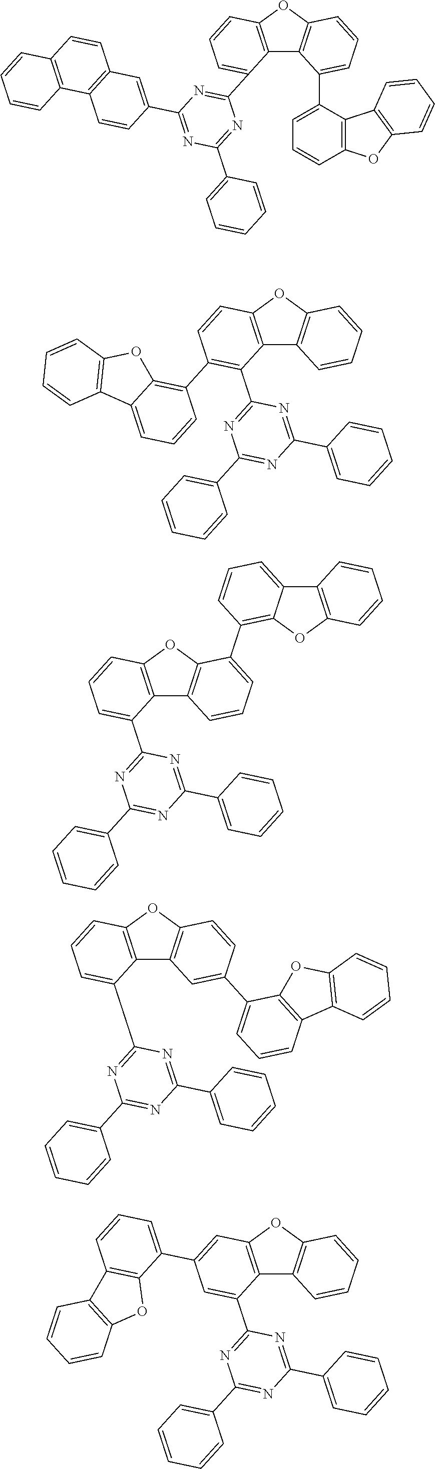

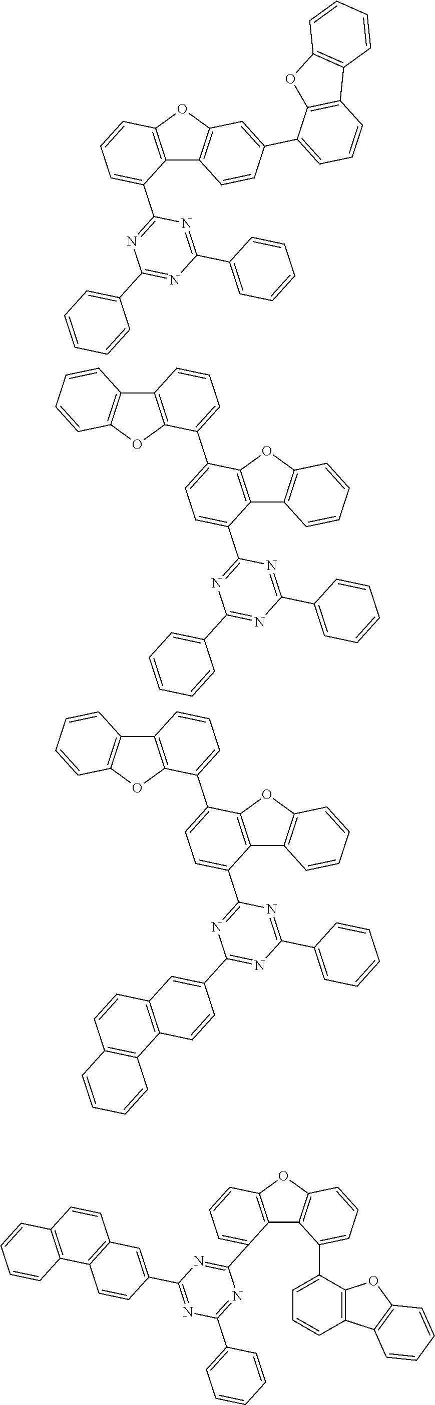

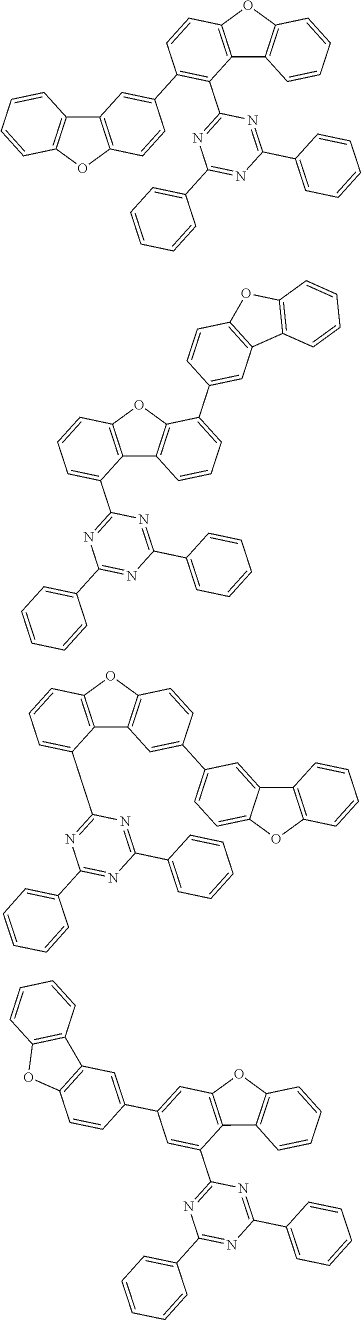

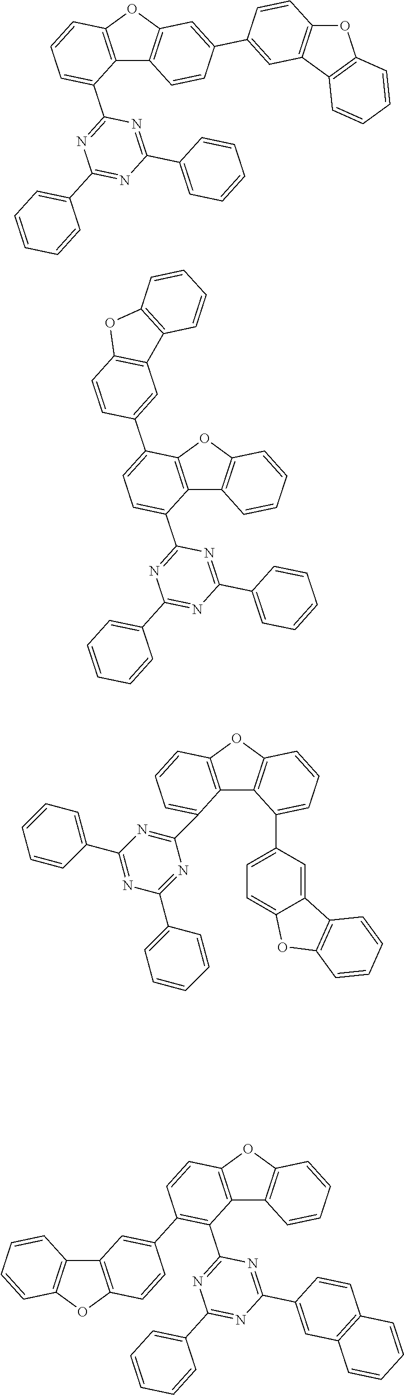 [SMILES: c1ccc(-c2nc(-c3ccccc3)nc(-c3cccc4oc5cc(-c6ccc7oc8ccccc8c7c6)ccc5c34)n2)cc1 ; c1ccc(-c2nc(-c3ccccc3)nc(-c3ccc(-c4ccc5oc6ccccc6c5c4)c4oc5ccccc5c34)n2)cc1 ; c1ccc(-c2nc(-c3ccccc3)nc(-c3cccc4oc5cccc(-c6ccc7oc8ccccc8c7c6)c5c34)n2)cc1 ; c1ccc(-c2nc(-c3ccc4ccccc4c3)nc(-c3c(-c4ccc5oc6ccccc6c5c4)ccc4oc5ccccc5c34)n2)cc1]

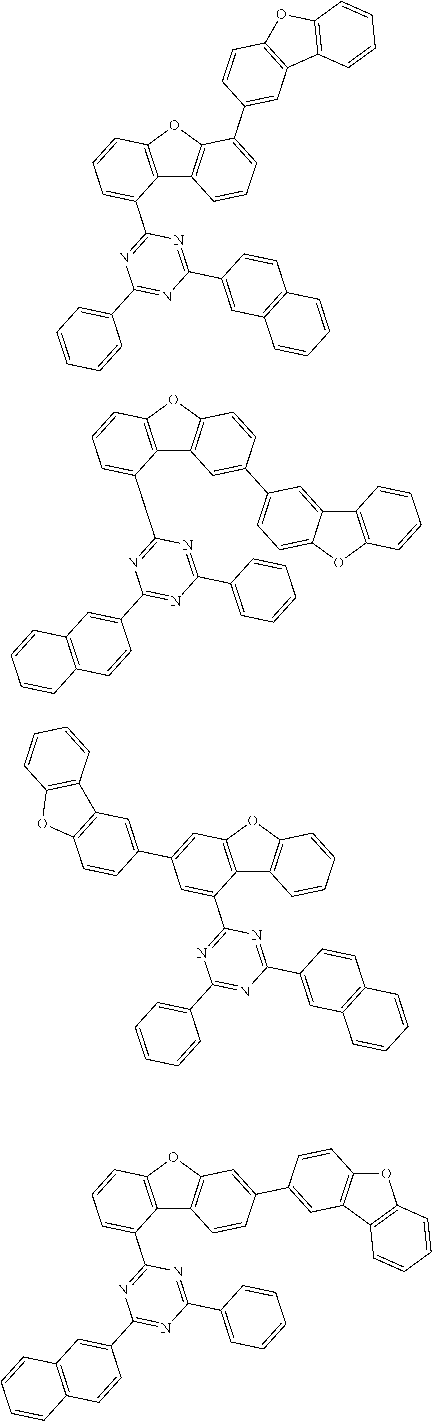

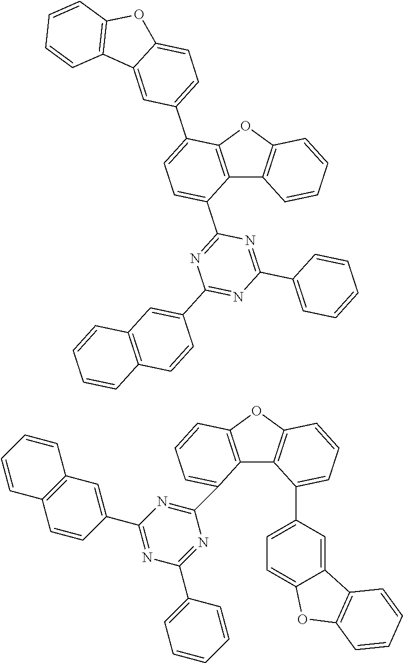 [SMILES: c1ccc(-c2nc(-c3ccc4ccccc4c3)nc(-c3ccc(-c4ccc5oc6ccccc6c5c4)c4oc5ccccc5c34)n2)cc1 ; c1ccc(-c2nc(-c3ccc4ccccc4c3)nc(-c3cccc4oc5cccc(-c6ccc7oc8ccccc8c7c6)c5c34)n2)cc1]

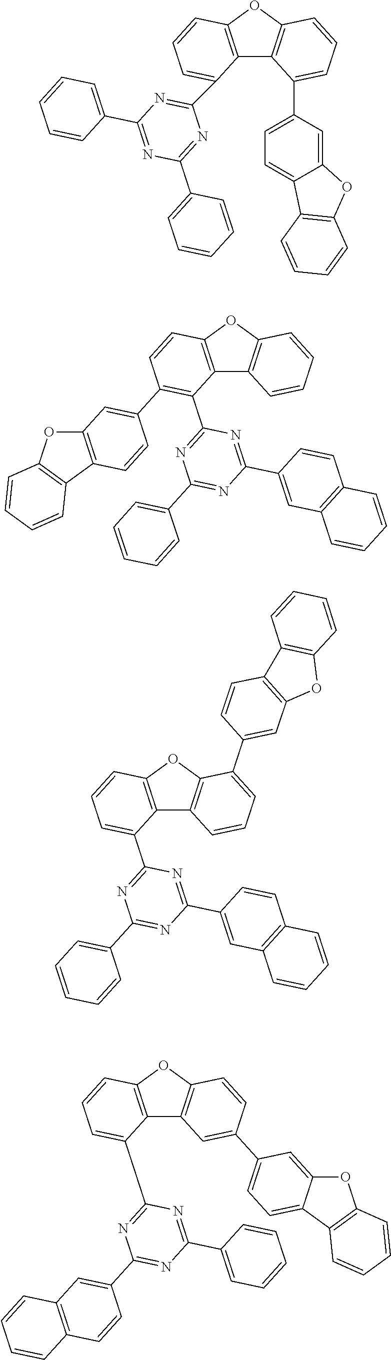 [SMILES: c1ccc(-c2nc(-c3ccccc3)nc(-c3cccc4oc5cccc(-c6ccc7c(c6)oc6ccccc67)c5c34)n2)cc1 ; c1ccc(-c2nc(-c3ccc4ccccc4c3)nc(-c3c(-c4ccc5c(c4)oc4ccccc45)ccc4oc5ccccc5c34)n2)cc1 ; c1ccc(-c2nc(-c3ccc4ccccc4c3)nc(-c3cccc4oc5c(-c6ccc7c(c6)oc6ccccc67)cccc5c34)n2)cc1 ; c1ccc(-c2nc(-c3ccc4ccccc4c3)nc(-c3cccc4oc5ccc(-c6ccc7c(c6)oc6ccccc67)cc5c34)n2)cc1]

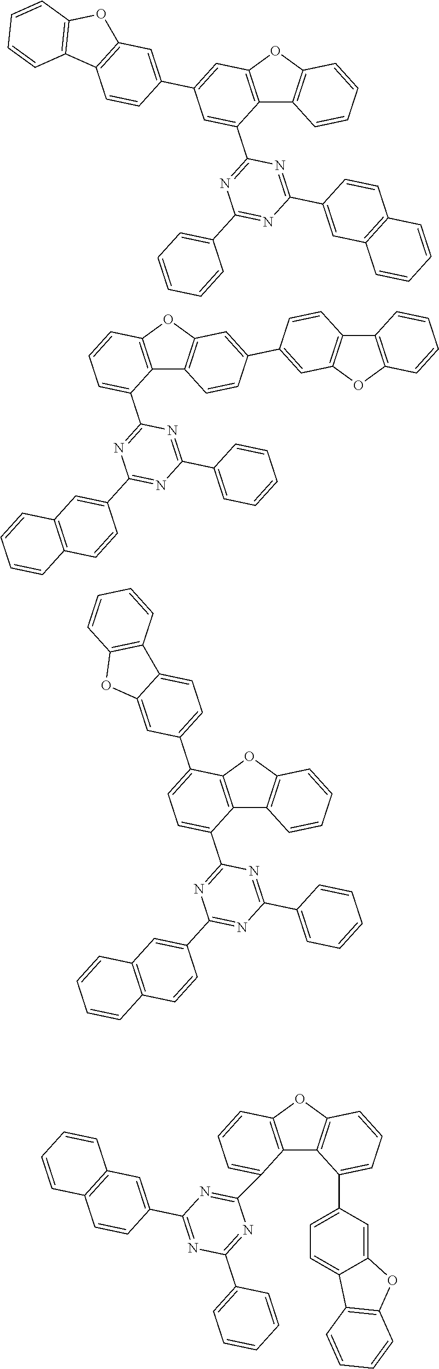

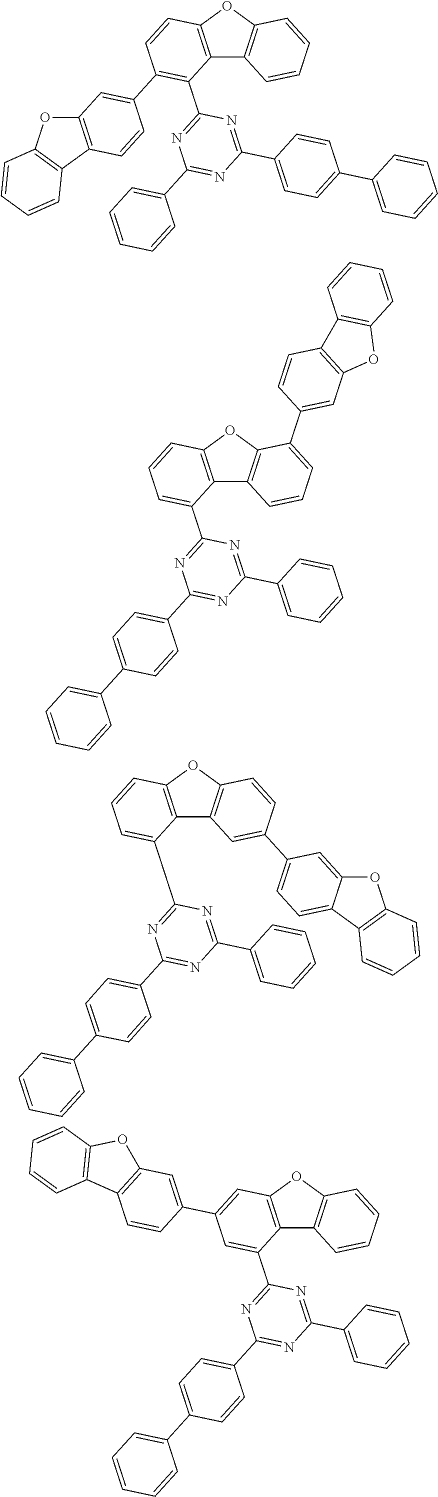

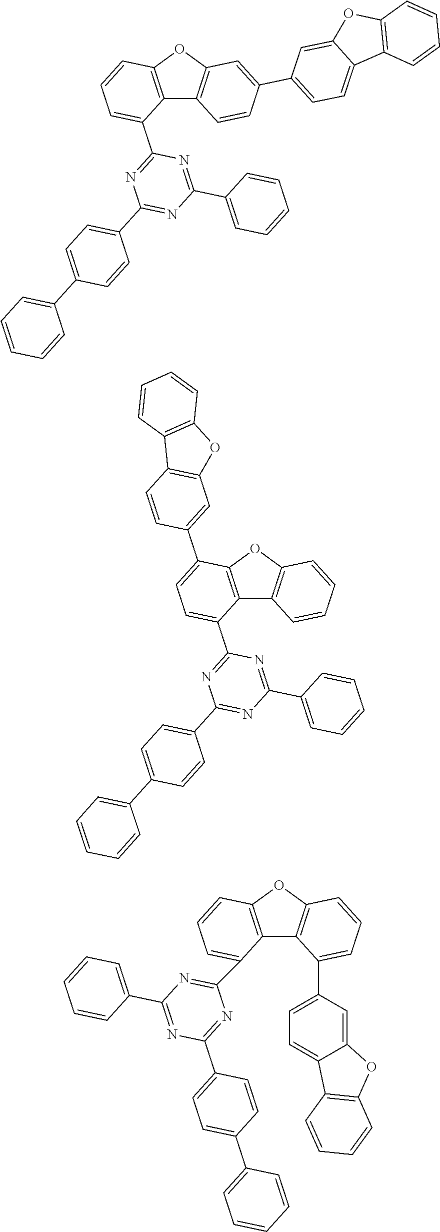 [SMILES: c1ccc(-c2ccc(-c3nc(-c4ccccc4)nc(-c4cccc5oc6cc(-c7ccc8c(c7)oc7ccccc78)ccc6c45)n3)cc2)cc1 ; c1ccc(-c2ccc(-c3nc(-c4ccccc4)nc(-c4ccc(-c5ccc6c(c5)oc5ccccc56)c5oc6ccccc6c45)n3)cc2)cc1 ; c1ccc(-c2ccc(-c3nc(-c4ccccc4)nc(-c4cccc5oc6cccc(-c7ccc8c(c7)oc7ccccc78)c6c45)n3)cc2)cc1]

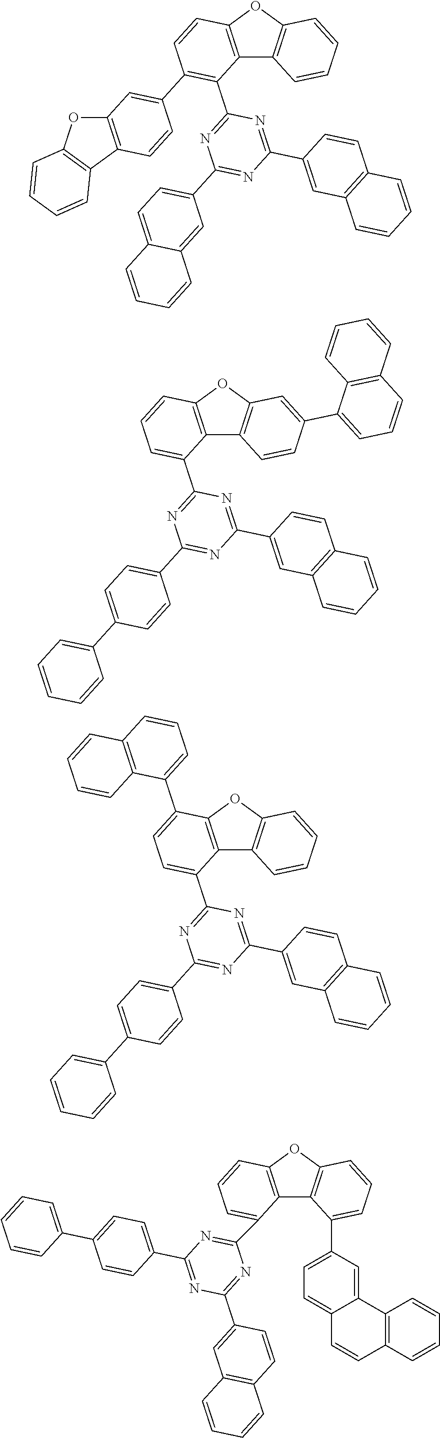

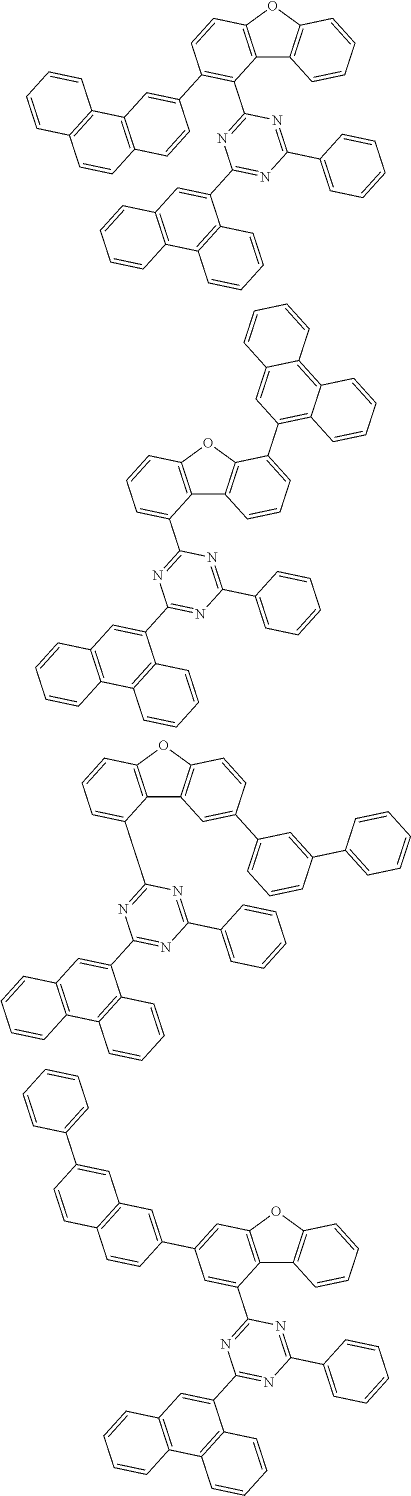

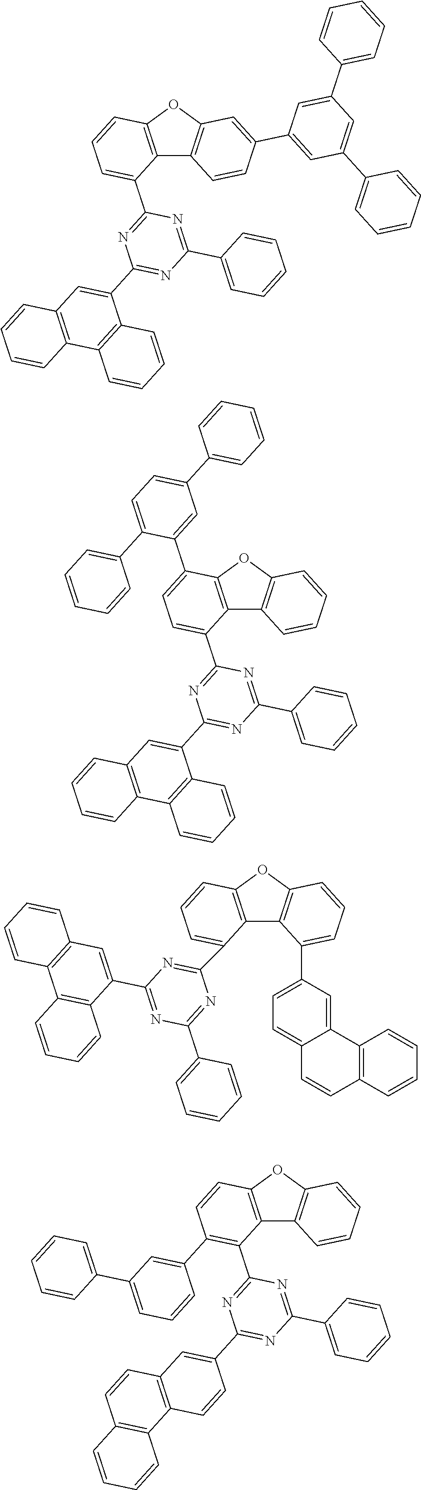

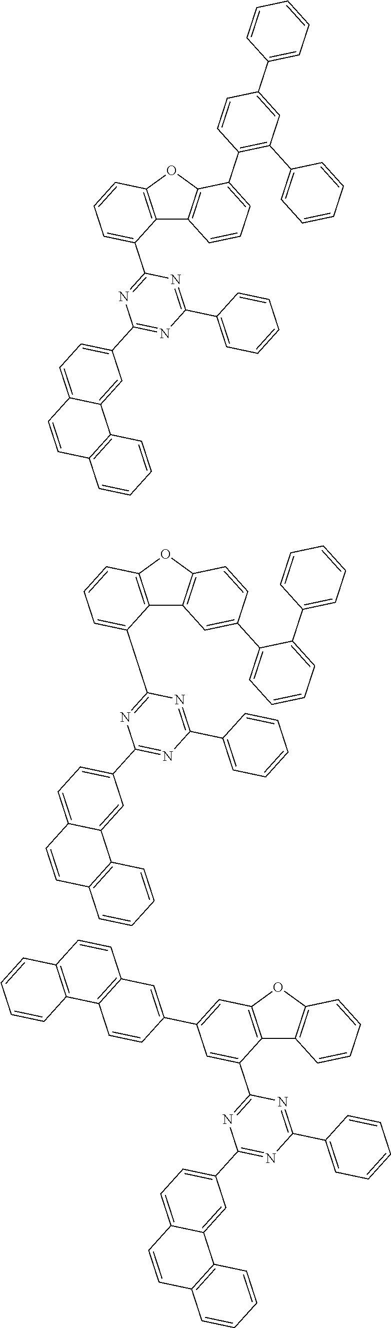

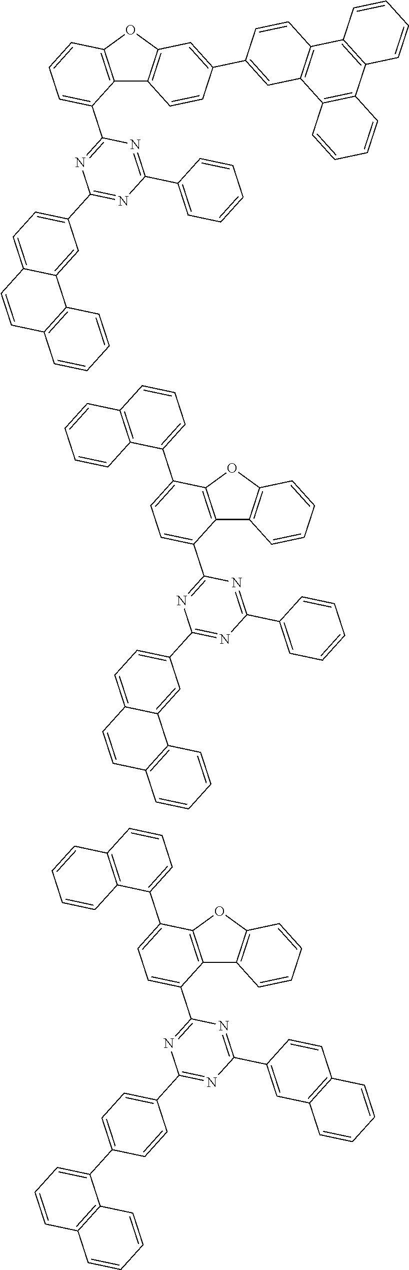

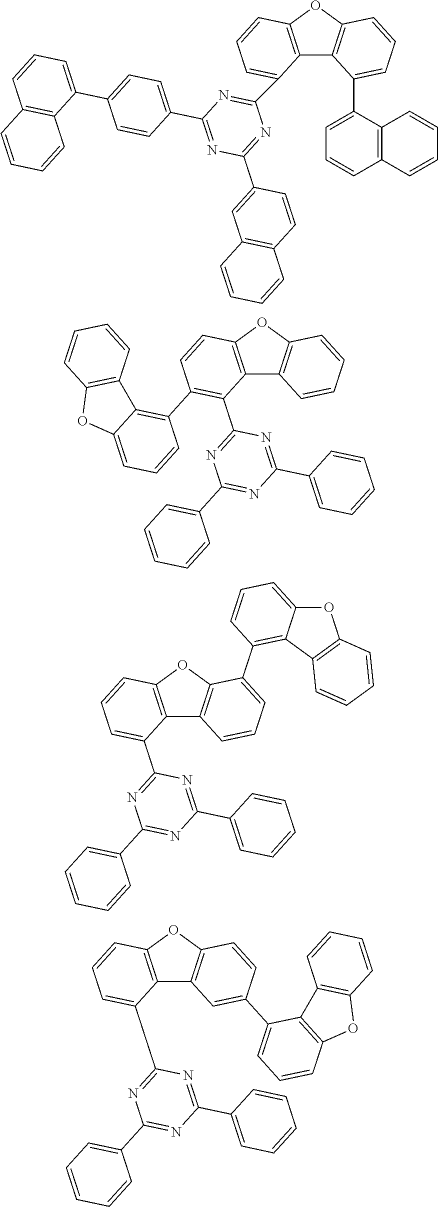

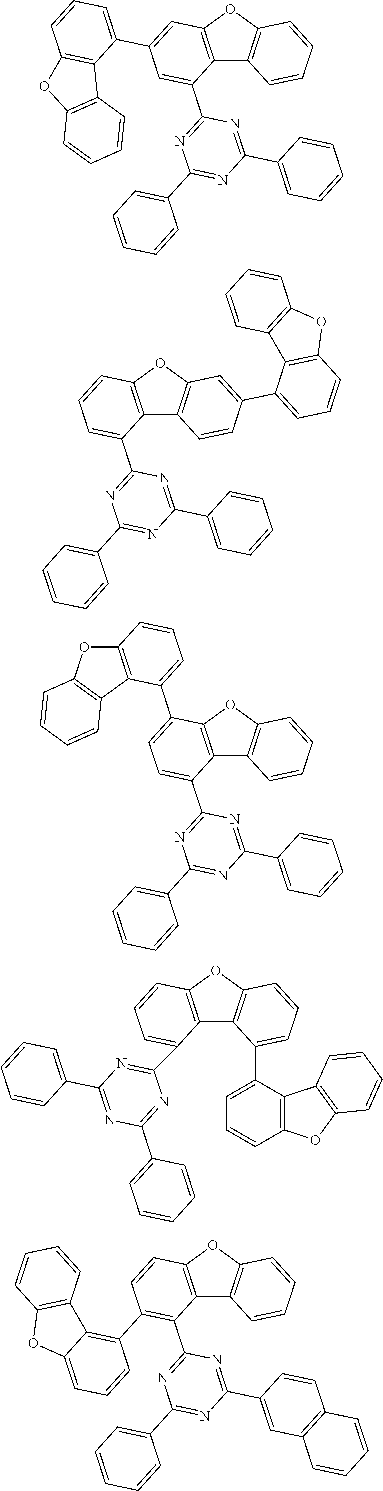

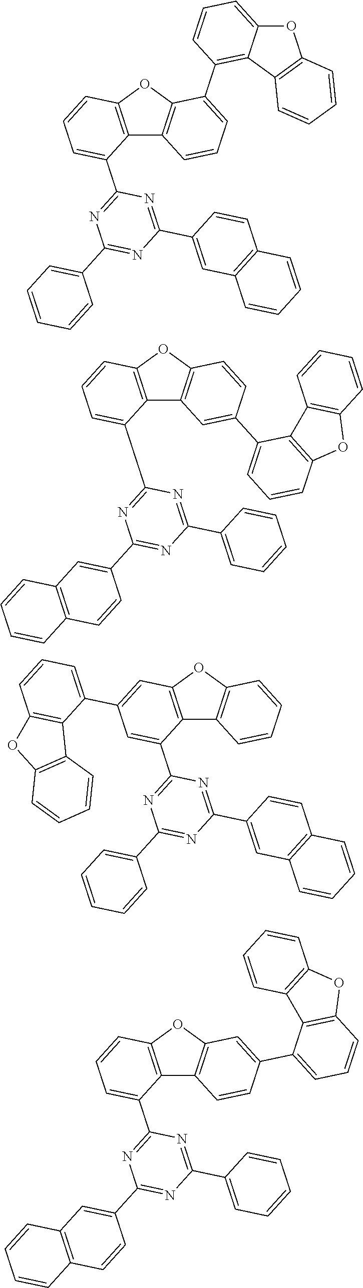

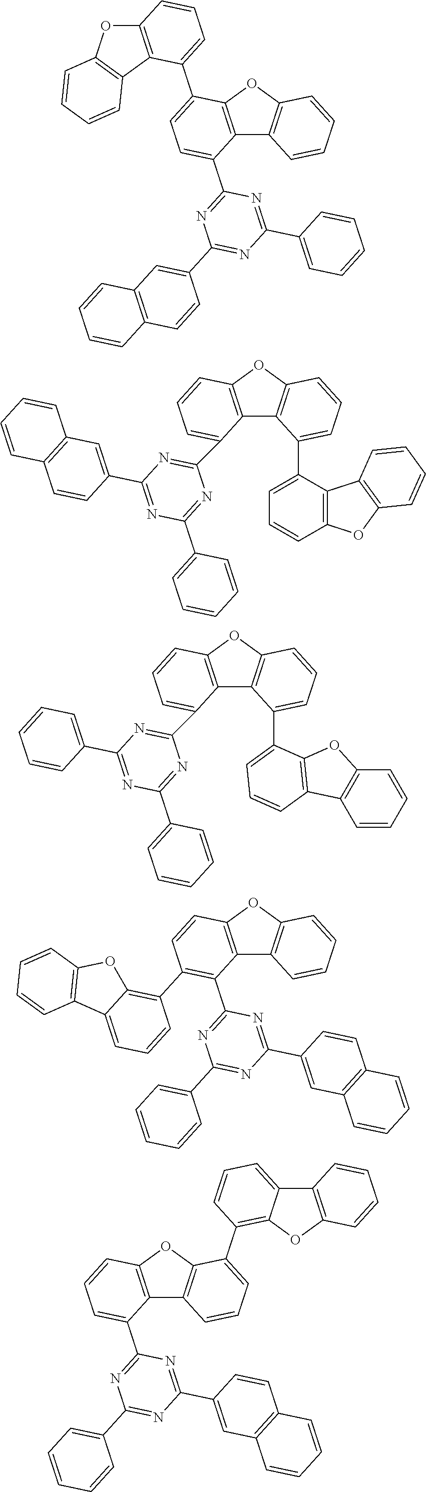

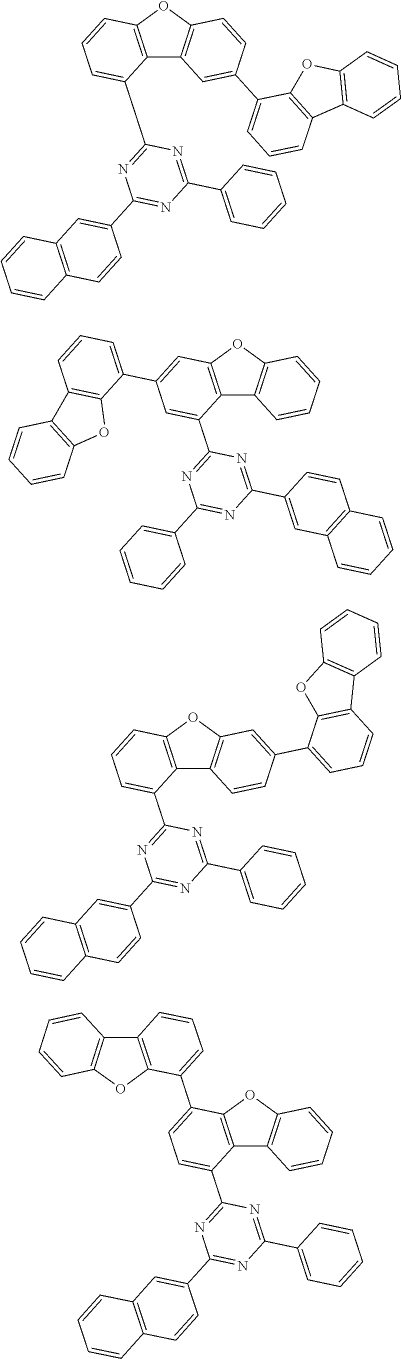

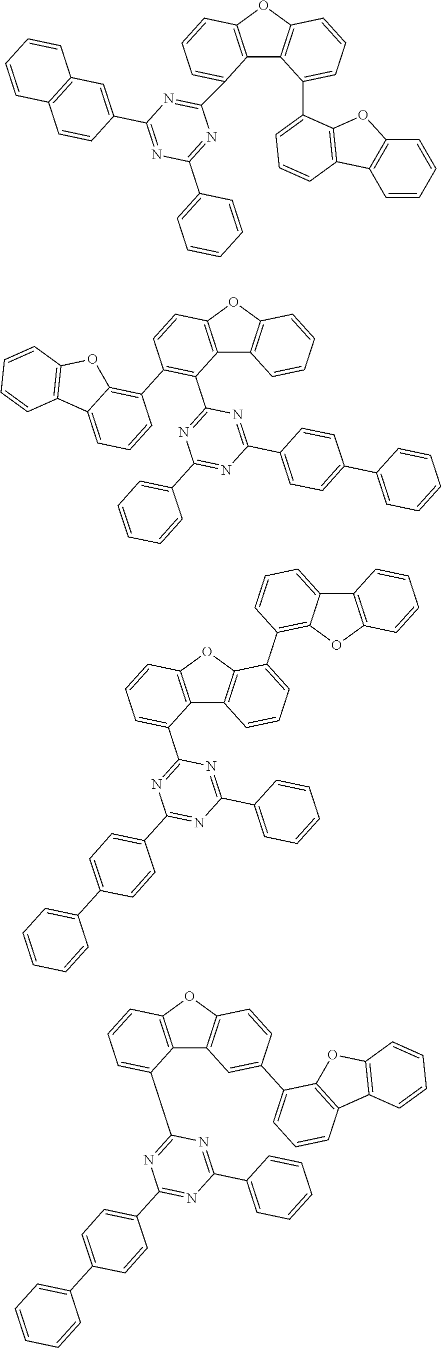 [SMILES: c1ccc(-c2nc(-c3ccc4ccccc4c3)nc(-c3cccc4oc5cccc(-c6cccc7c6oc6ccccc67)c5c34)n2)cc1 ; c1ccc(-c2ccc(-c3nc(-c4ccccc4)nc(-c4c(-c5cccc6c5oc5ccccc56)ccc5oc6ccccc6c45)n3)cc2)cc1 ; c1ccc(-c2ccc(-c3nc(-c4ccccc4)nc(-c4cccc5oc6c(-c7cccc8c7oc7ccccc78)cccc6c45)n3)cc2)cc1 ; c1ccc(-c2ccc(-c3nc(-c4ccccc4)nc(-c4cccc5oc6ccc(-c7cccc8c7oc7ccccc78)cc6c45)n3)cc2)cc1]

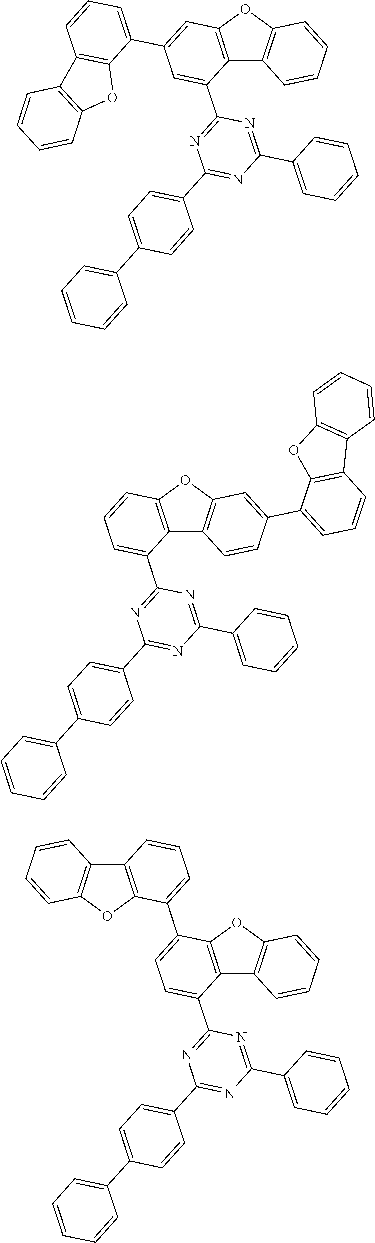 [SMILES: c1ccc(-c2ccc(-c3nc(-c4ccccc4)nc(-c4cc(-c5cccc6c5oc5ccccc56)cc5oc6ccccc6c45)n3)cc2)cc1 ; c1ccc(-c2ccc(-c3nc(-c4ccccc4)nc(-c4cccc5oc6cc(-c7cccc8c7oc7ccccc78)ccc6c45)n3)cc2)cc1 ; c1ccc(-c2ccc(-c3nc(-c4ccccc4)nc(-c4ccc(-c5cccc6c5oc5ccccc56)c5oc6ccccc6c45)n3)cc2)cc1]

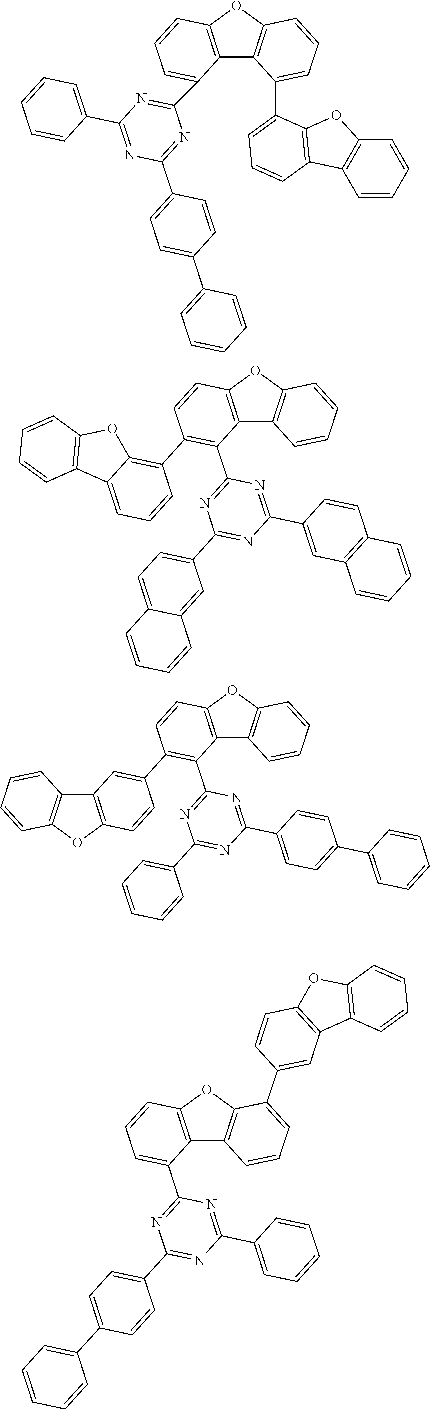

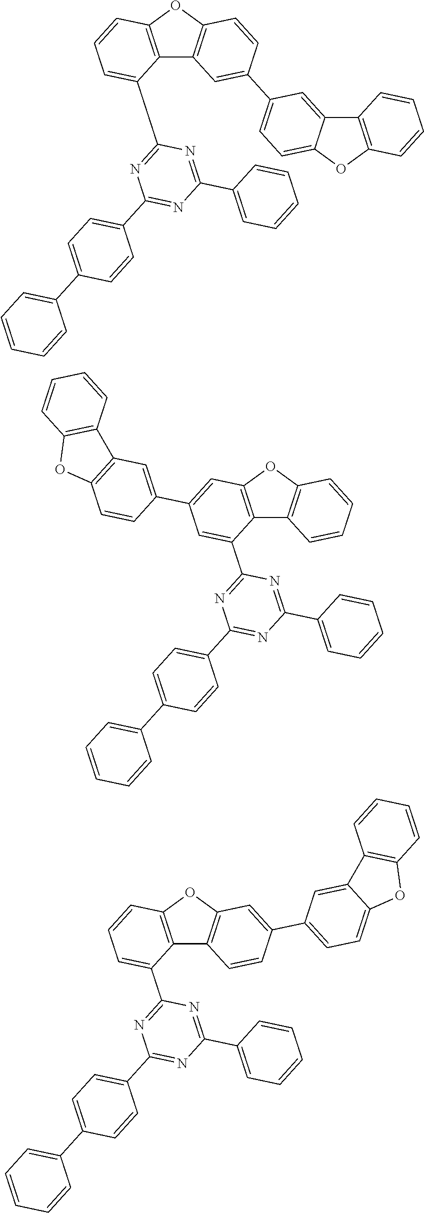

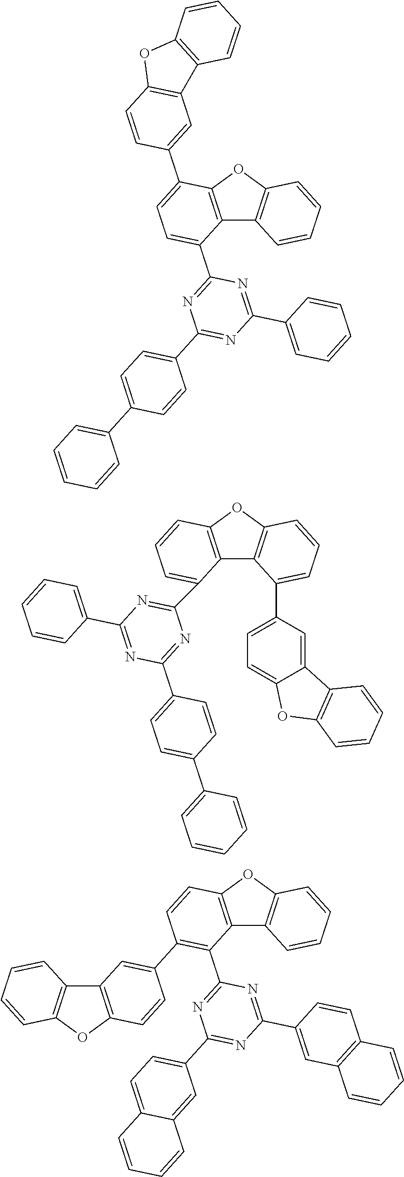

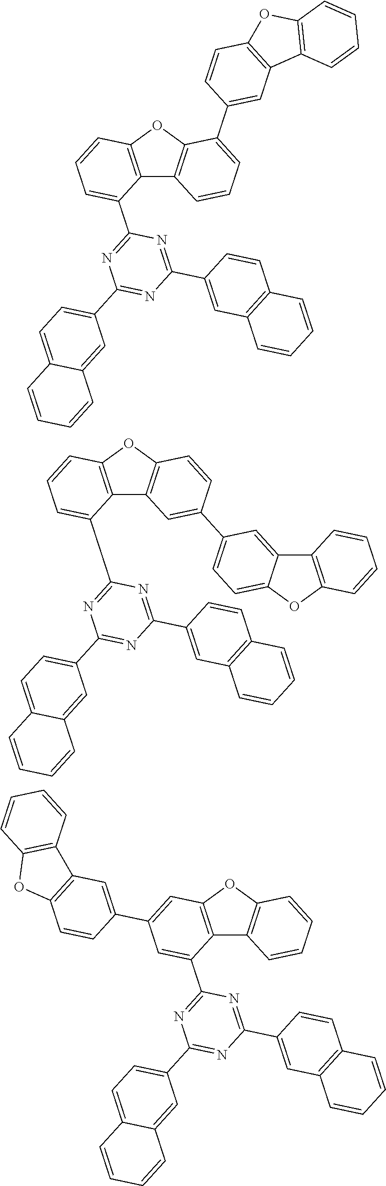

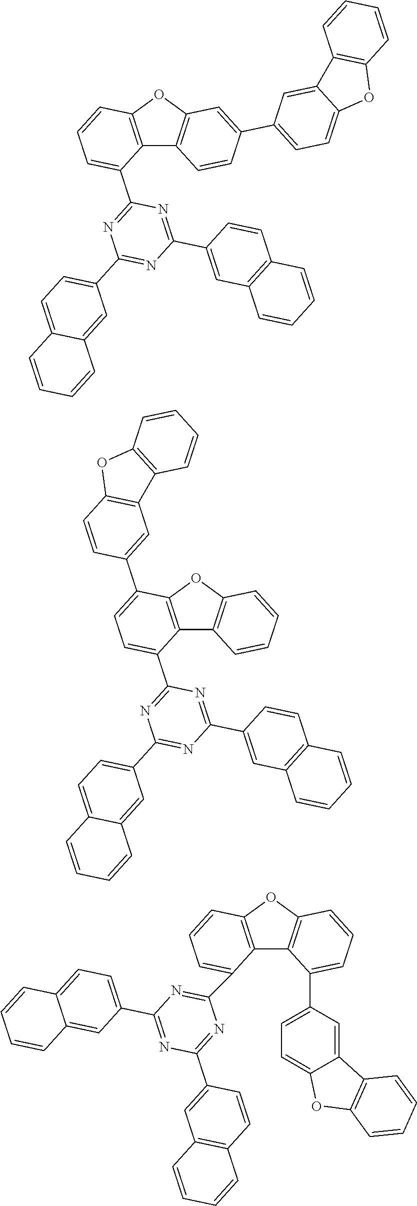

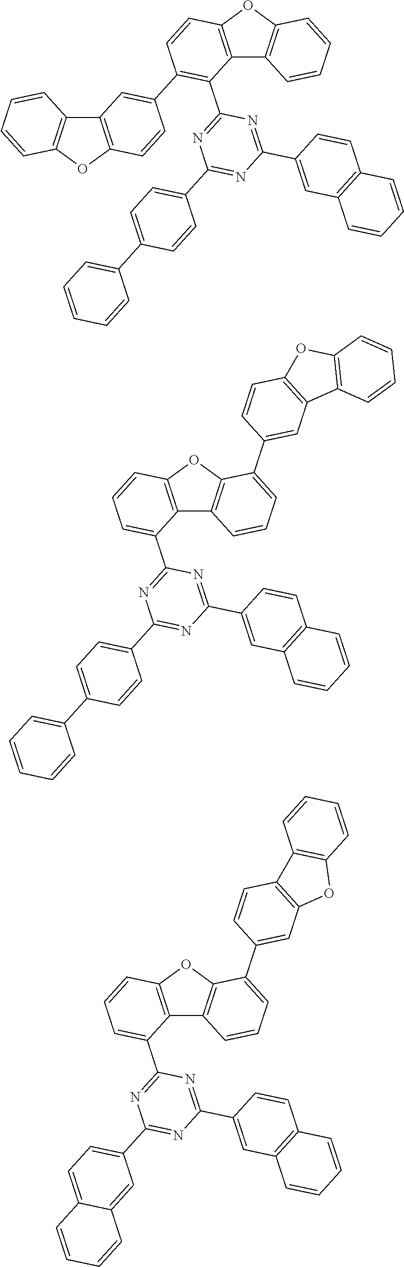 [SMILES: c1ccc(-c2ccc(-c3nc(-c4ccc5ccccc5c4)nc(-c4c(-c5ccc6oc7ccccc7c6c5)ccc5oc6ccccc6c45)n3)cc2)cc1 ; c1ccc(-c2ccc(-c3nc(-c4ccc5ccccc5c4)nc(-c4cccc5oc6c(-c7ccc8oc9ccccc9c8c7)cccc6c45)n3)cc2)cc1 ; c1ccc2cc(-c3nc(-c4ccc5ccccc5c4)nc(-c4cccc5oc6c(-c7ccc8c(c7)oc7ccccc78)cccc6c45)n3)ccc2c1]

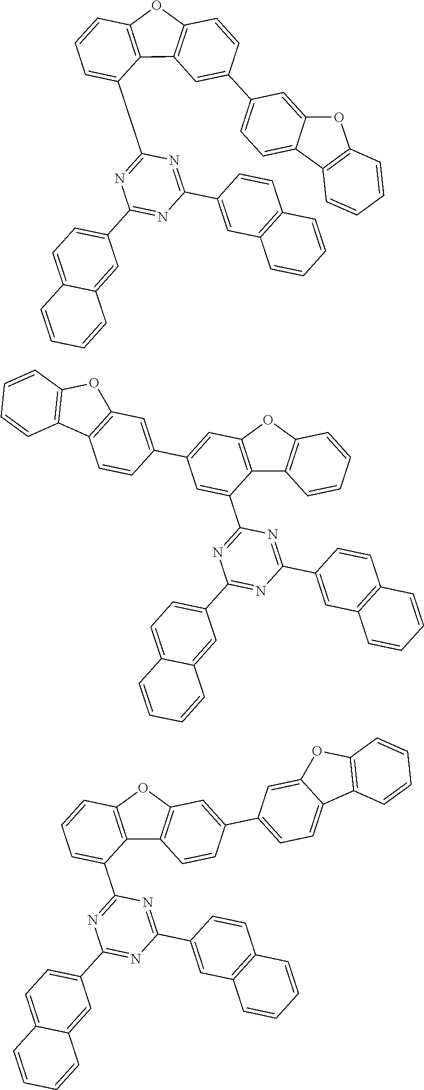

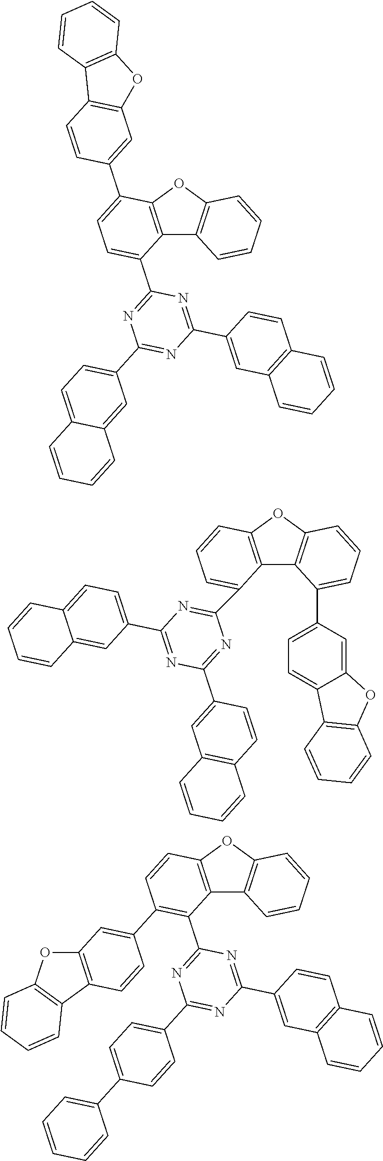

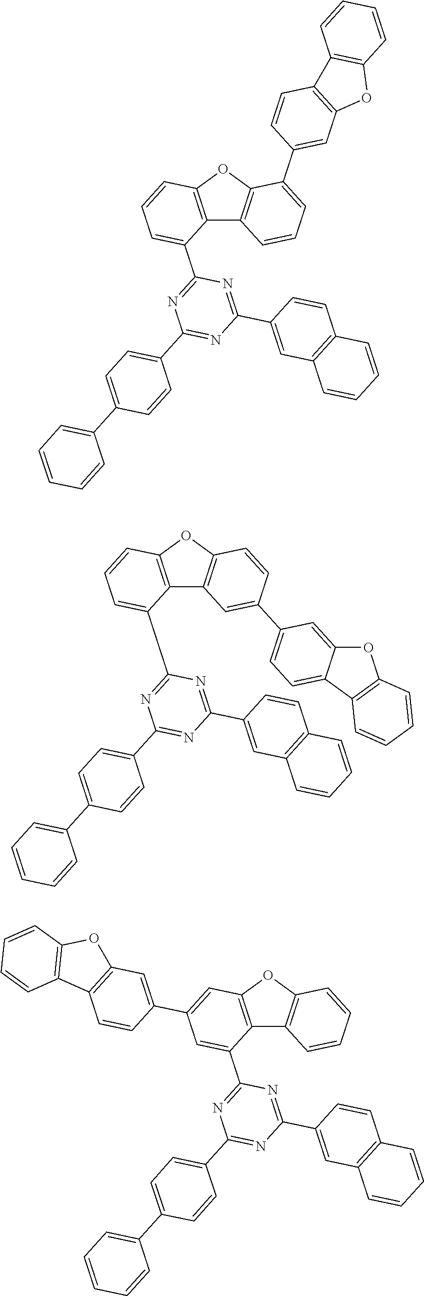

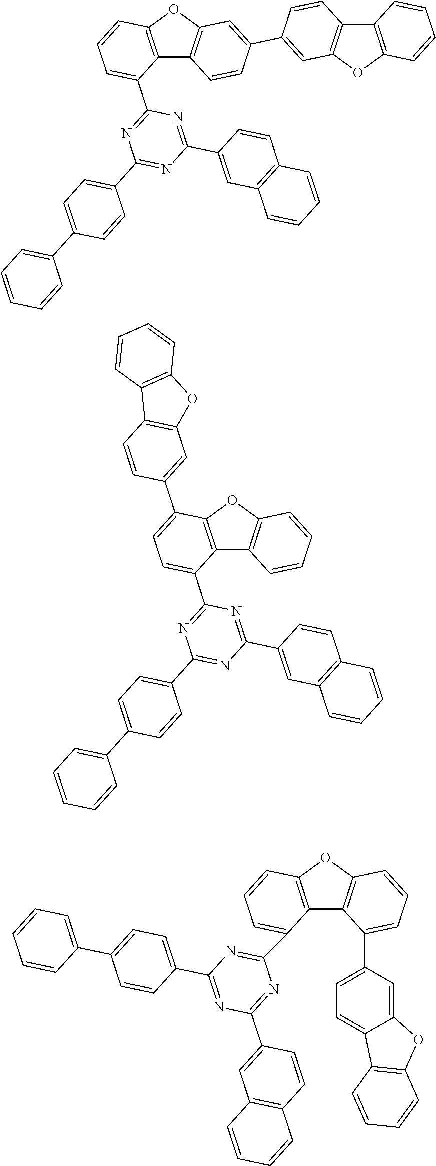 [SMILES: c1ccc(-c2ccc(-c3nc(-c4ccc5ccccc5c4)nc(-c4cccc5oc6cc(-c7ccc8c(c7)oc7ccccc78)ccc6c45)n3)cc2)cc1 ; c1ccc(-c2ccc(-c3nc(-c4ccc5ccccc5c4)nc(-c4ccc(-c5ccc6c(c5)oc5ccccc56)c5oc6ccccc6c45)n3)cc2)cc1 ; c1ccc(-c2ccc(-c3nc(-c4ccc5ccccc5c4)nc(-c4cccc5oc6cccc(-c7ccc8c(c7)oc7ccccc78)c6c45)n3)cc2)cc1]

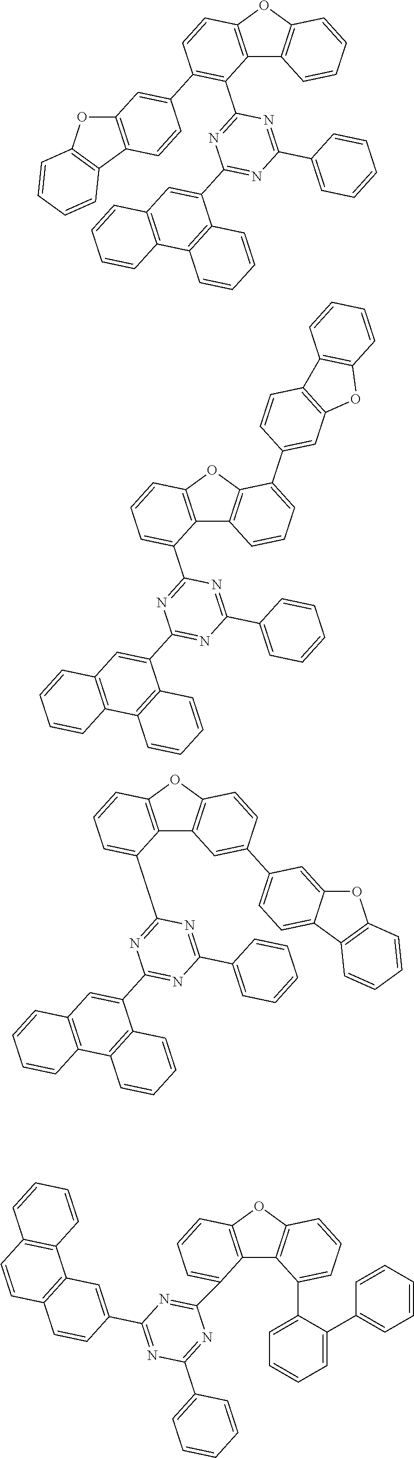

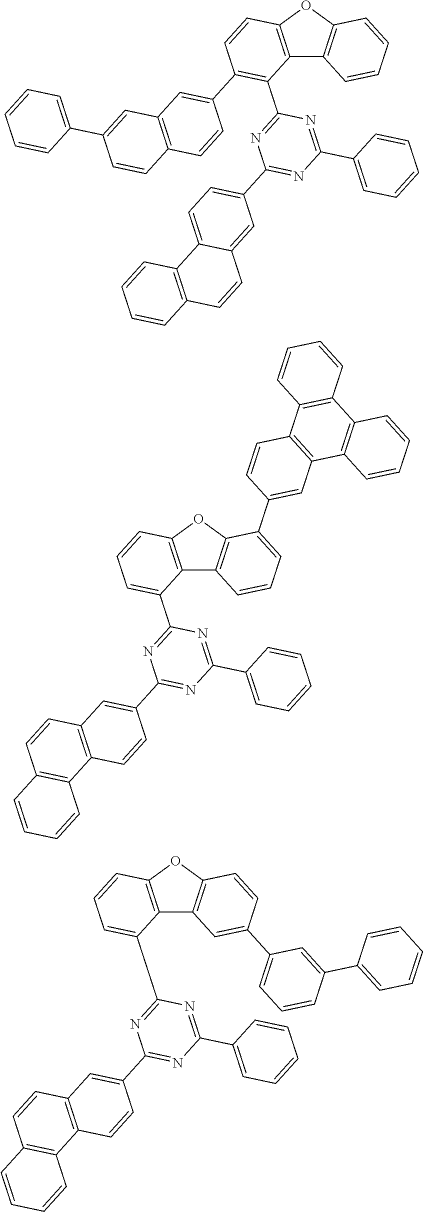 [SMILES: c1ccc(-c2ccc3ccc(-c4ccc5oc6ccccc6c5c4-c4nc(-c5ccccc5)nc(-c5ccc6c(ccc7ccccc76)c5)n4)cc3c2)cc1 ; c1ccc(-c2nc(-c3ccc4c(ccc5ccccc54)c3)nc(-c3cccc4oc5c(-c6ccc7c8ccccc8c8ccccc8c7c6)cccc5c34)n2)cc1 ; c1ccc(-c2cccc(-c3ccc4oc5cccc(-c6nc(-c7ccccc7)nc(-c7ccc8c(ccc9ccccc98)c7)n6)c5c4c3)c2)cc1]

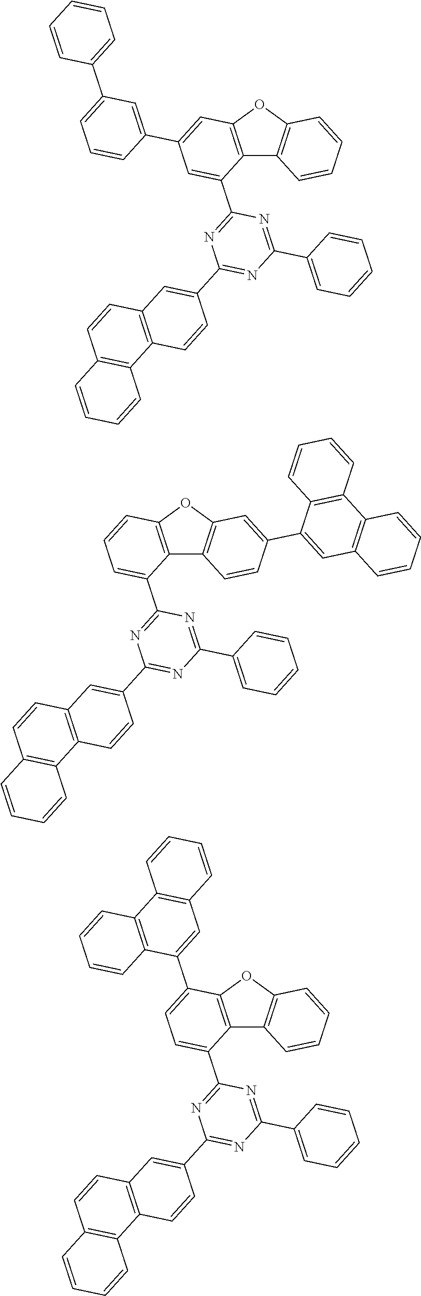 [SMILES: c1ccc(-c2cccc(-c3cc(-c4nc(-c5ccccc5)nc(-c5ccc6c(ccc7ccccc76)c5)n4)c4c(c3)oc3ccccc34)c2)cc1 ; c1ccc(-c2nc(-c3ccc4c(ccc5ccccc54)c3)nc(-c3cccc4oc5cc(-c6cc7ccccc7c7ccccc67)ccc5c34)n2)cc1 ; c1ccc(-c2nc(-c3ccc4c(ccc5ccccc54)c3)nc(-c3ccc(-c4cc5ccccc5c5ccccc45)c4oc5ccccc5c34)n2)cc1]

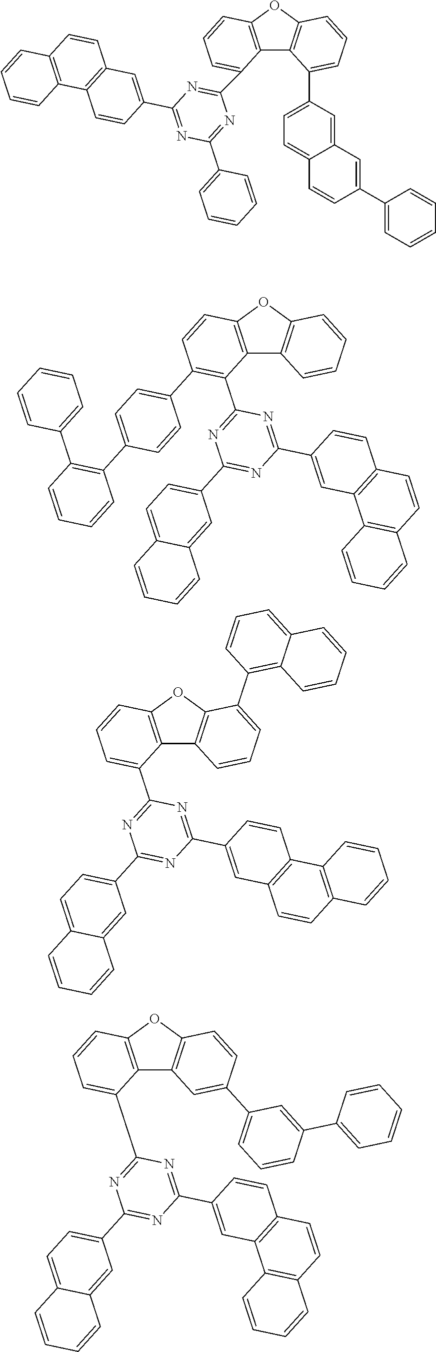

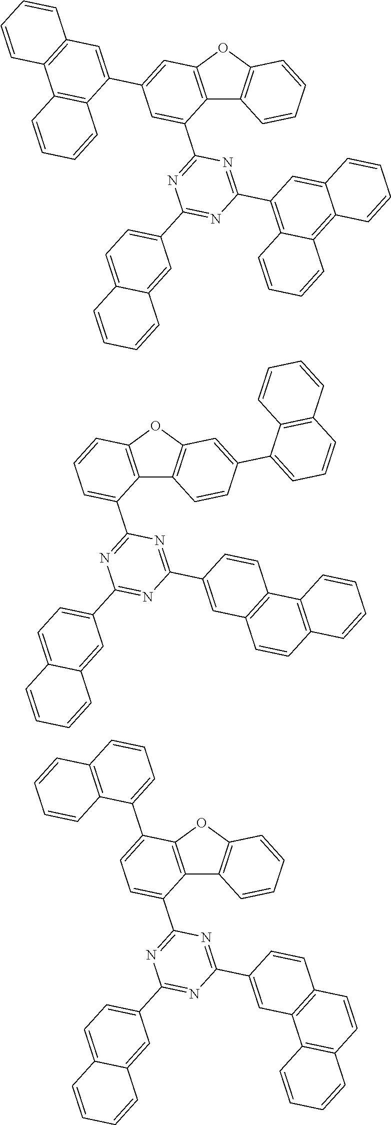

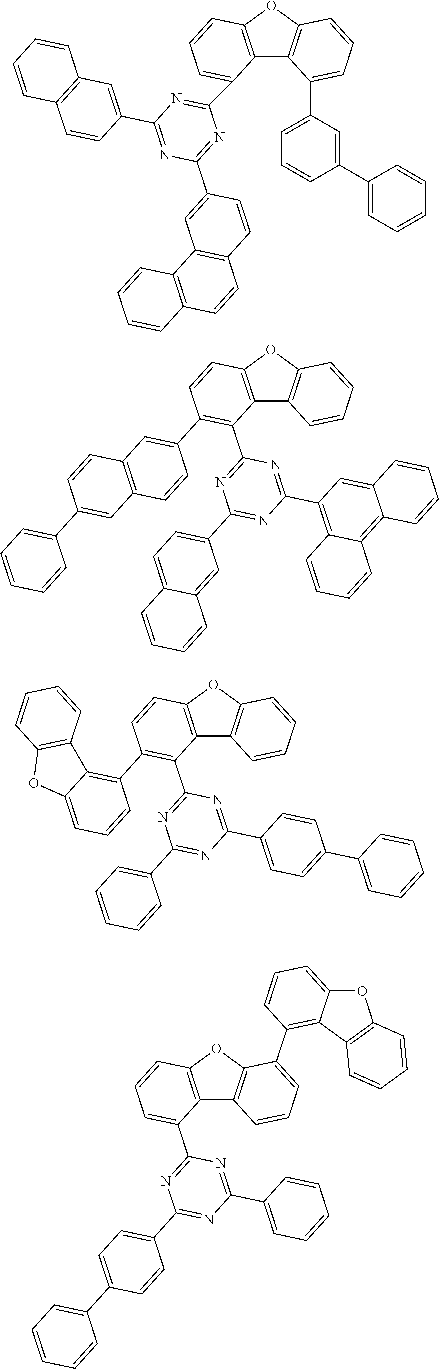

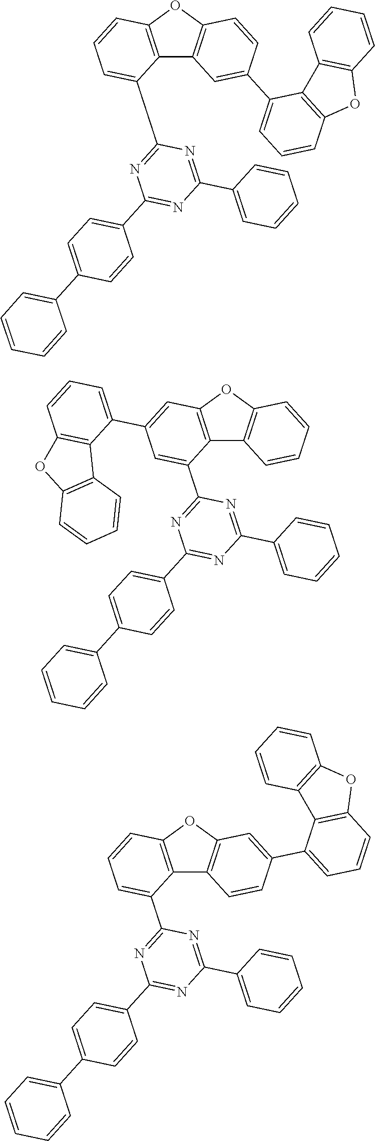

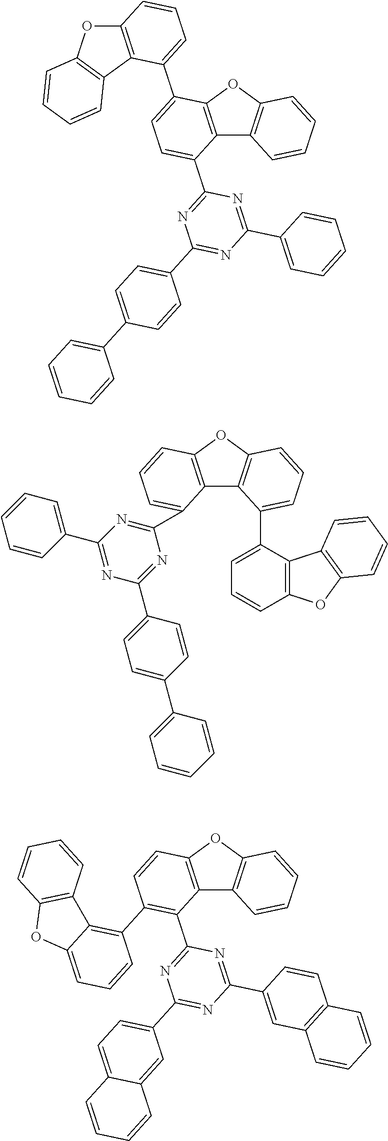 [SMILES: c1ccc(-c2ccc(-c3nc(-c4ccccc4)nc(-c4ccc(-c5cccc6oc7ccccc7c56)c5oc6ccccc6c45)n3)cc2)cc1 ; c1ccc(-c2ccc(-c3nc(-c4ccccc4)nc(-c4cccc5oc6cccc(-c7cccc8oc9ccccc9c78)c6c45)n3)cc2)cc1 ; c1ccc2cc(-c3nc(-c4ccc5ccccc5c4)nc(-c4c(-c5cccc6oc7ccccc7c56)ccc5oc6ccccc6c45)n3)ccc2c1]

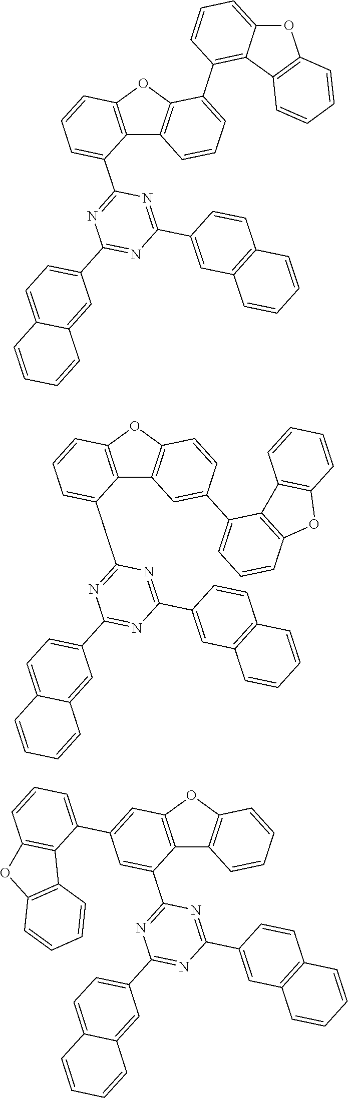

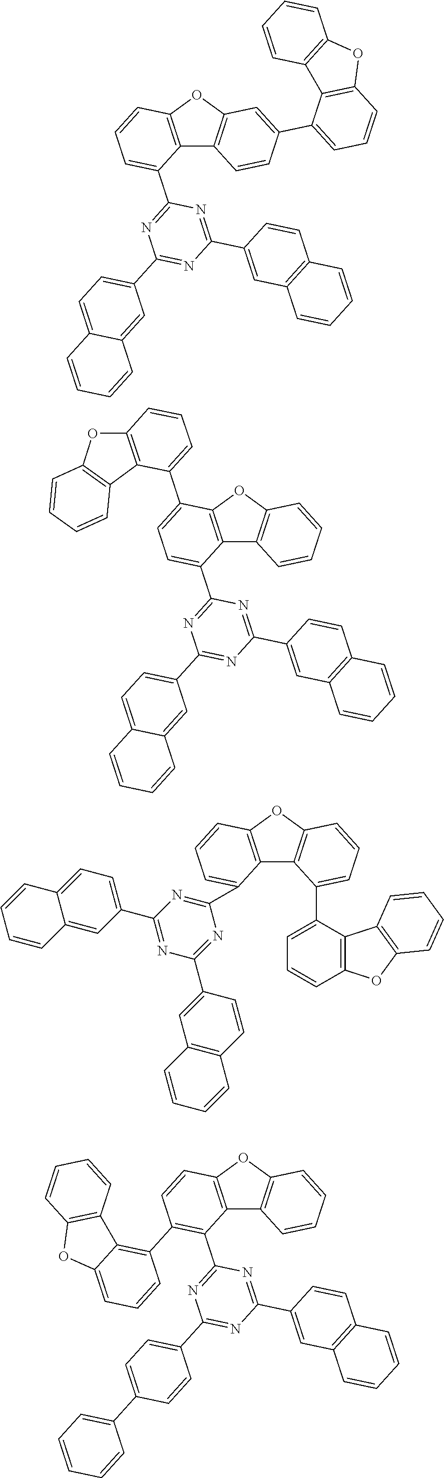

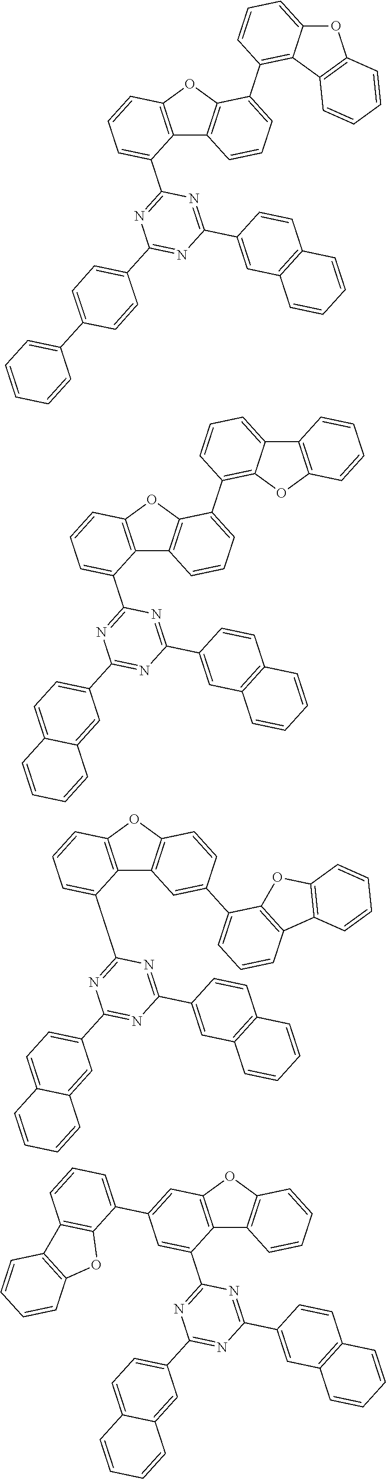

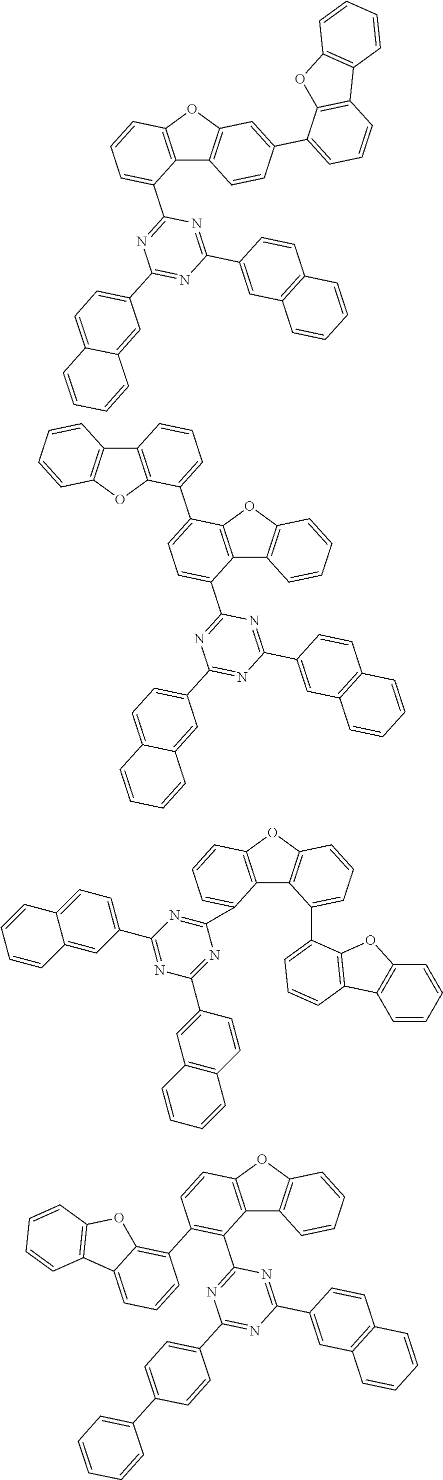

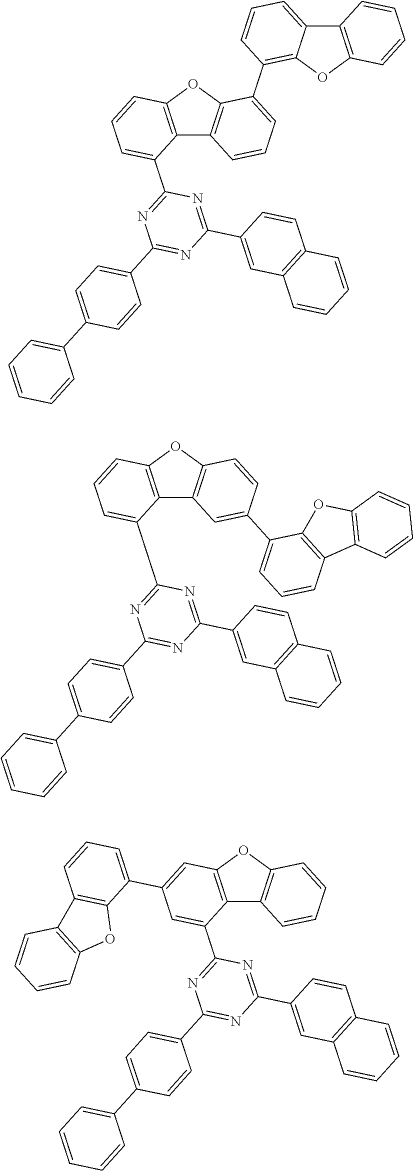 [SMILES: c1ccc(-c2ccc(-c3nc(-c4ccc5ccccc5c4)nc(-c4cccc5oc6c(-c7cccc8c7oc7ccccc78)cccc6c45)n3)cc2)cc1 ; c1ccc(-c2ccc(-c3nc(-c4ccc5ccccc5c4)nc(-c4cccc5oc6ccc(-c7cccc8c7oc7ccccc78)cc6c45)n3)cc2)cc1 ; c1ccc(-c2ccc(-c3nc(-c4ccc5ccccc5c4)nc(-c4cc(-c5cccc6c5oc5ccccc56)cc5oc6ccccc6c45)n3)cc2)cc1]

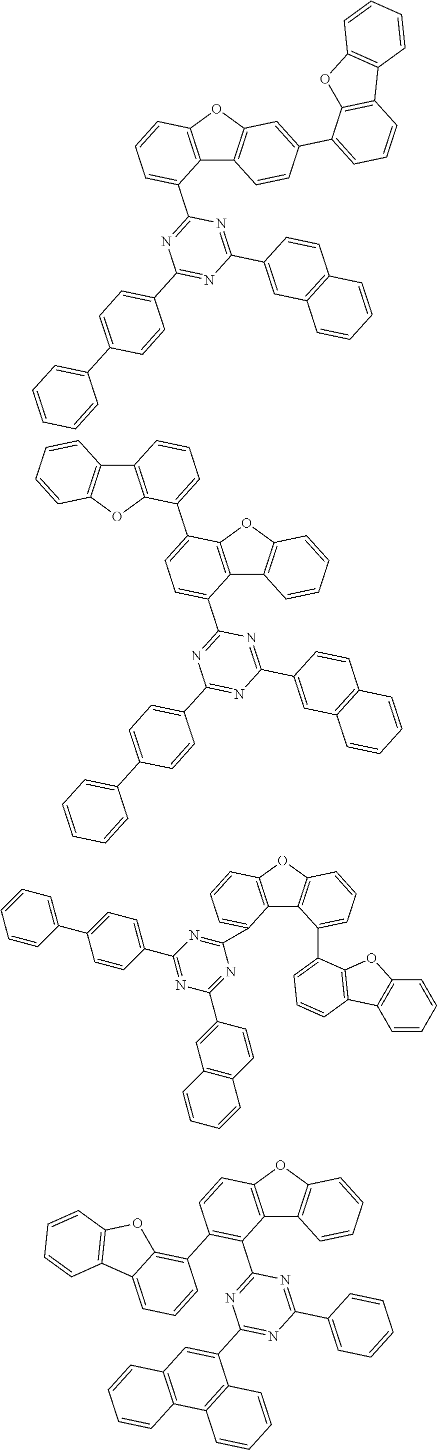

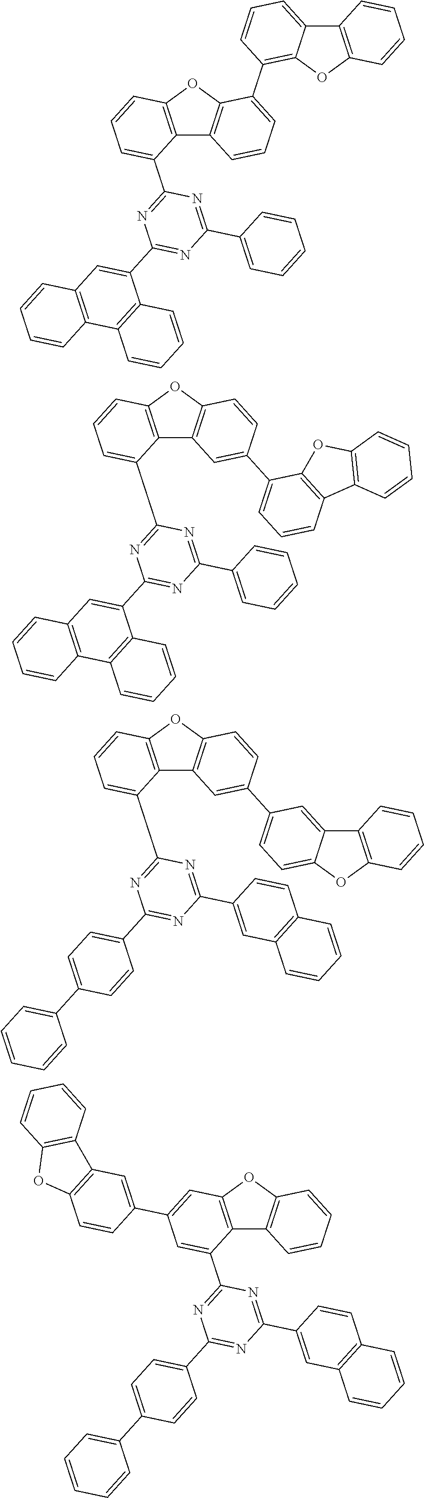

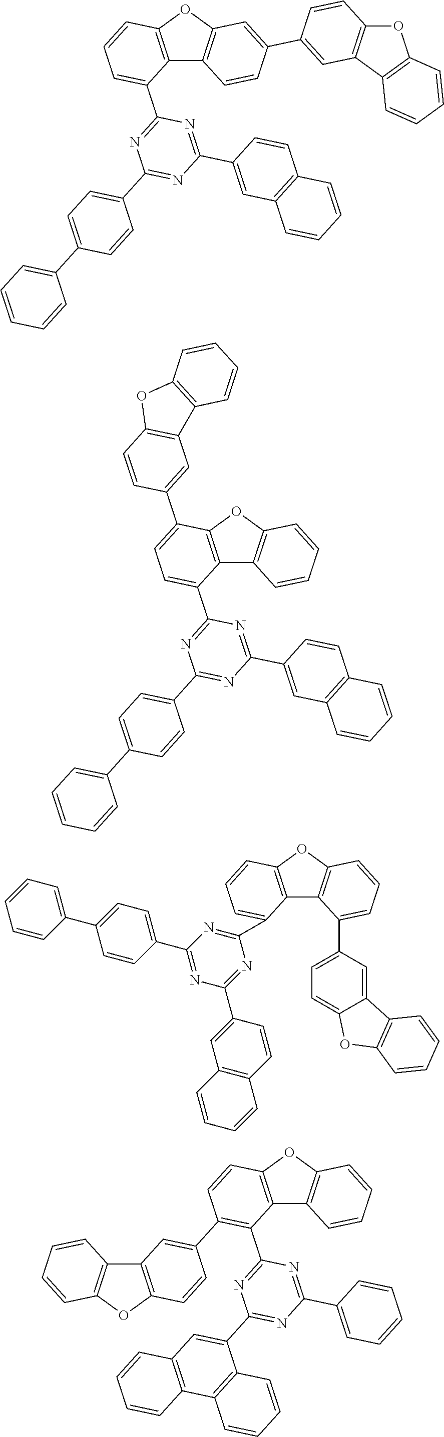

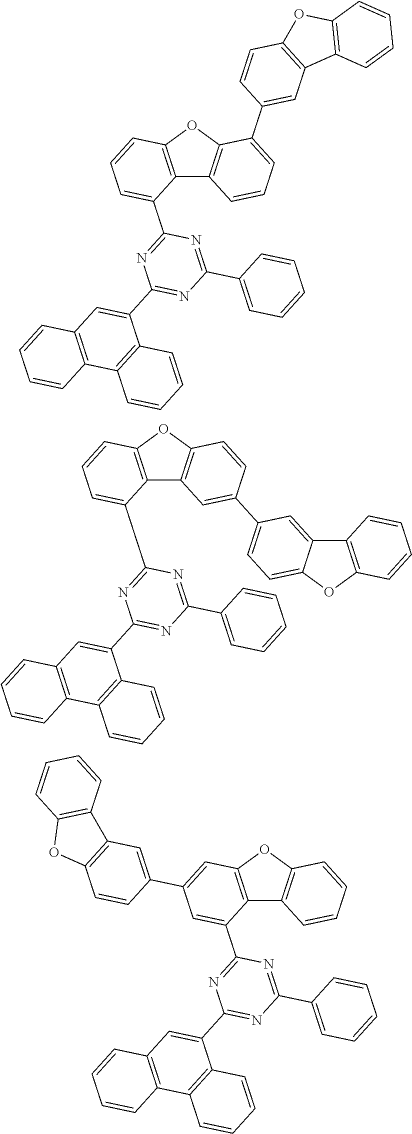

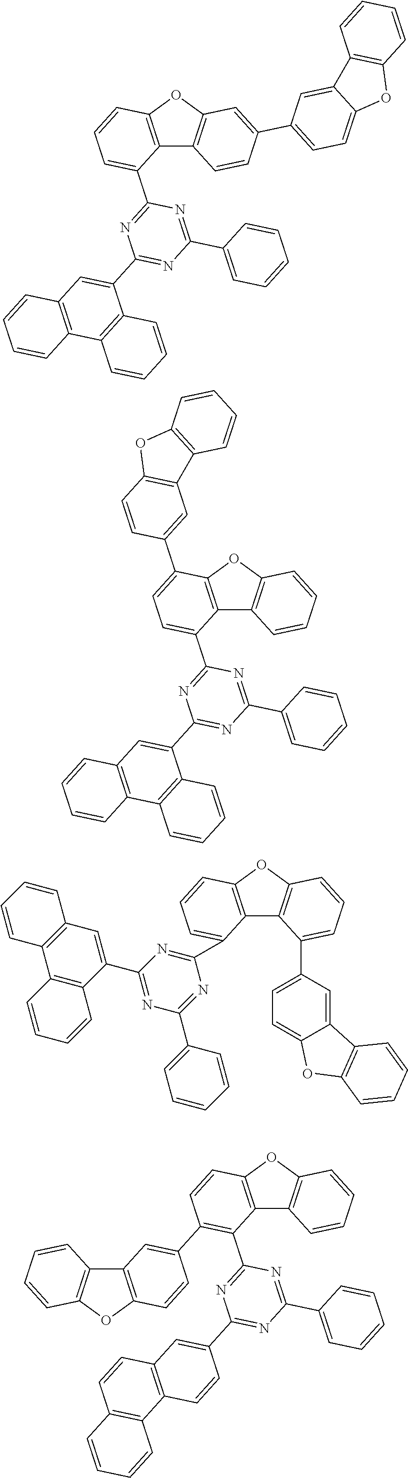

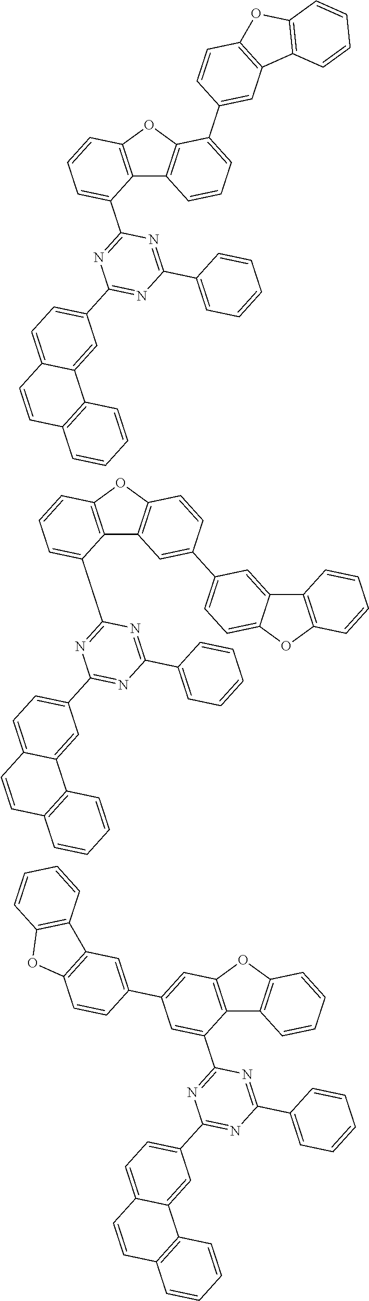

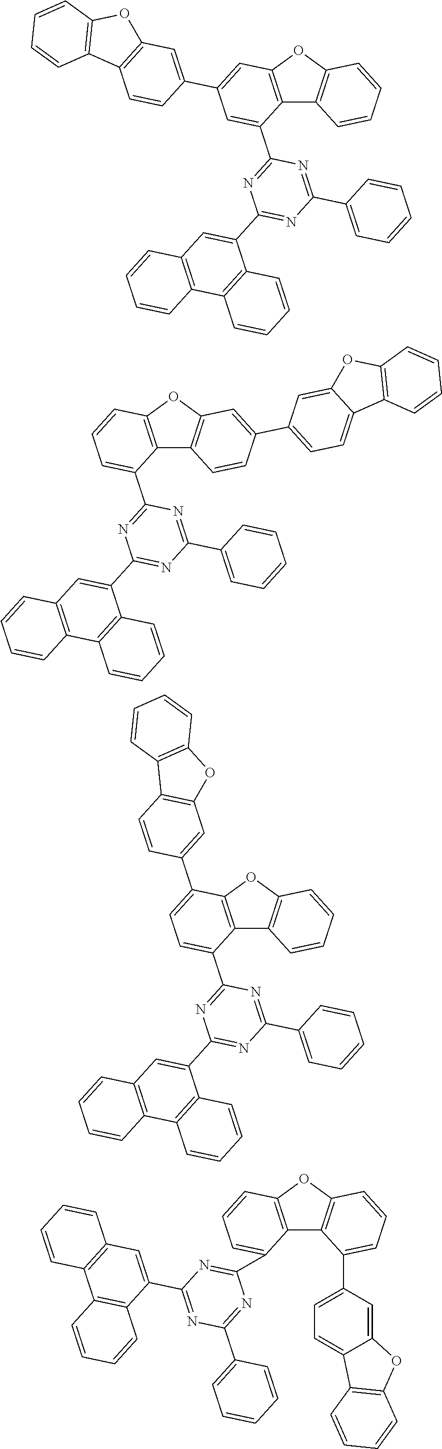

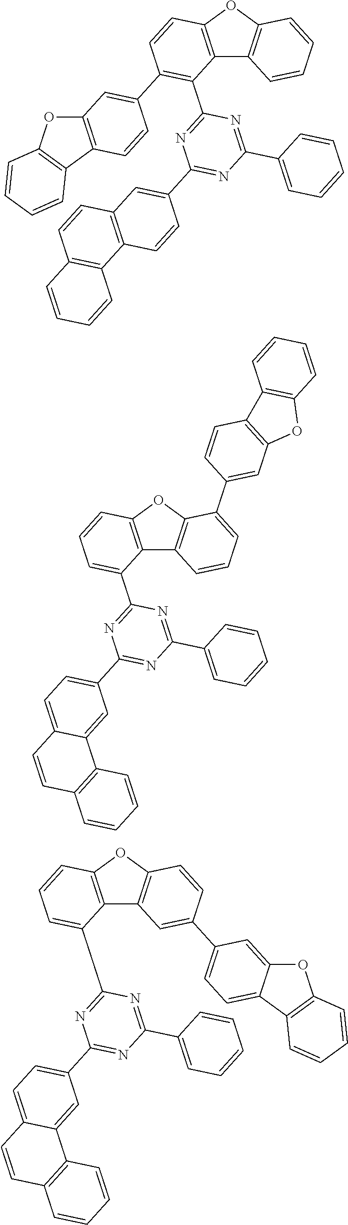

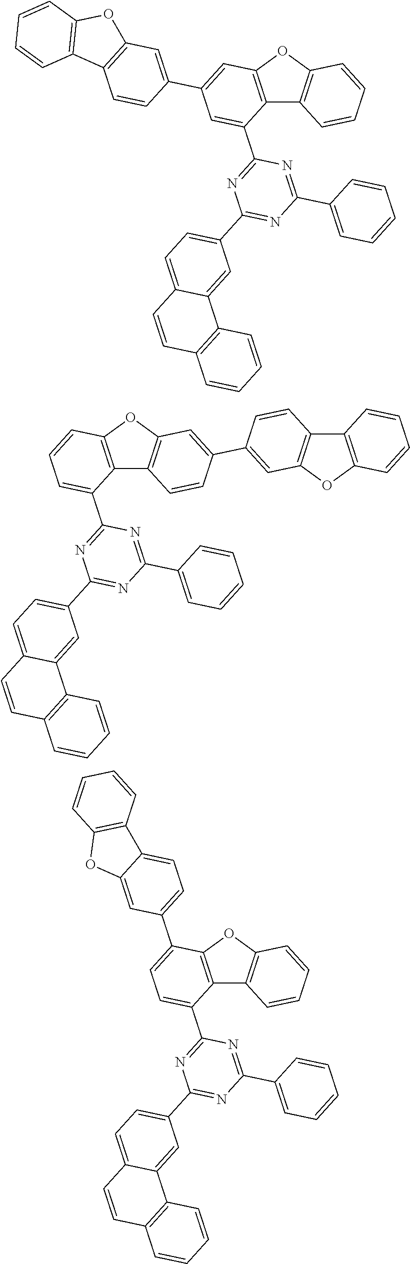

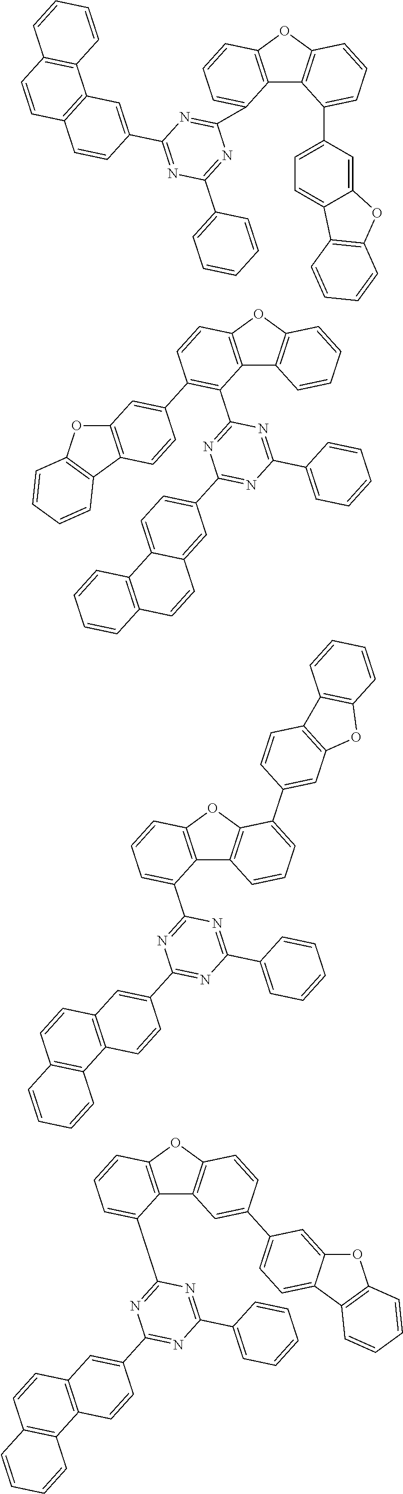

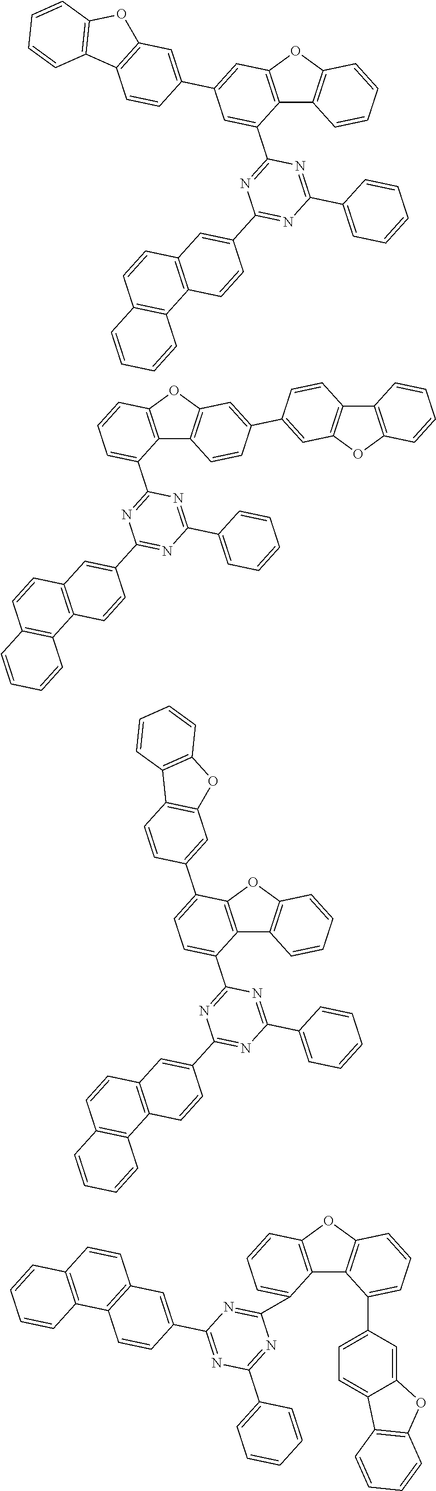

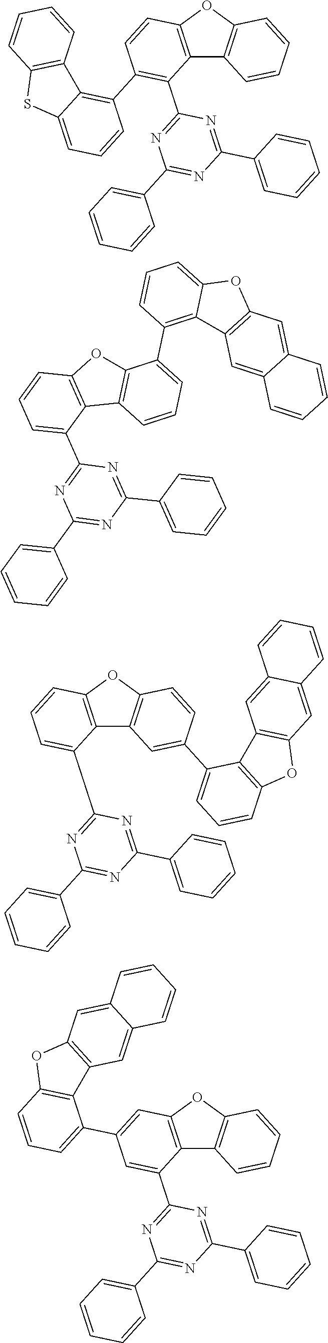

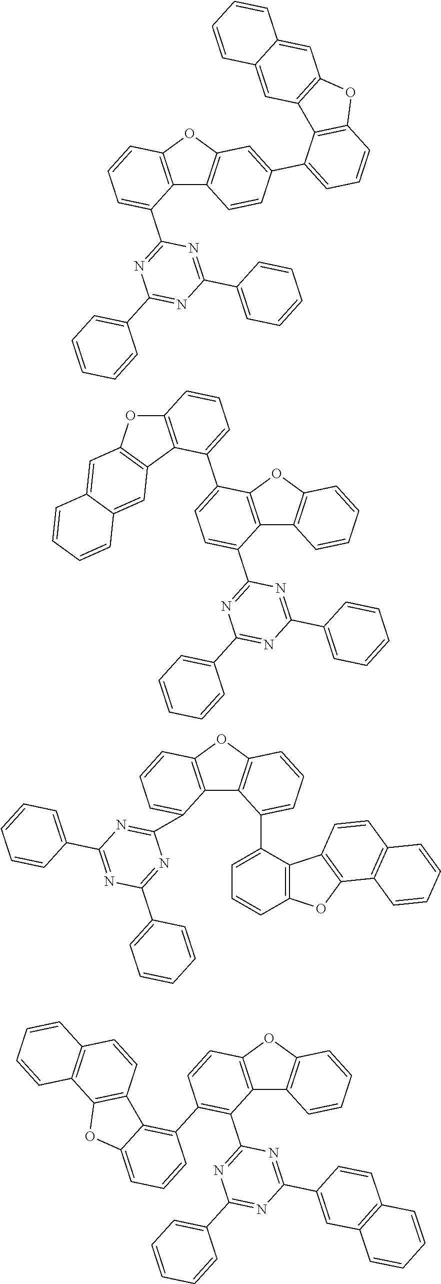

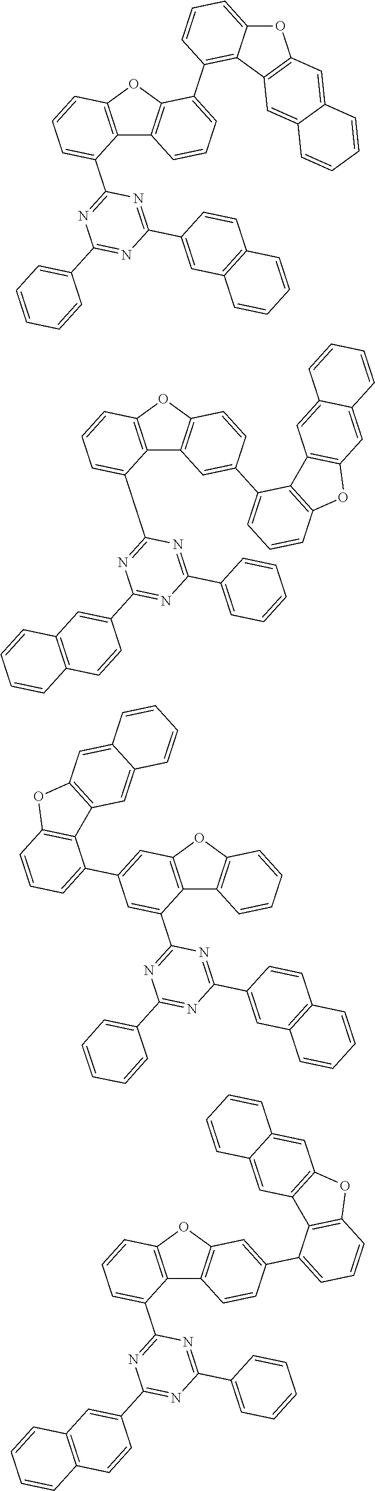

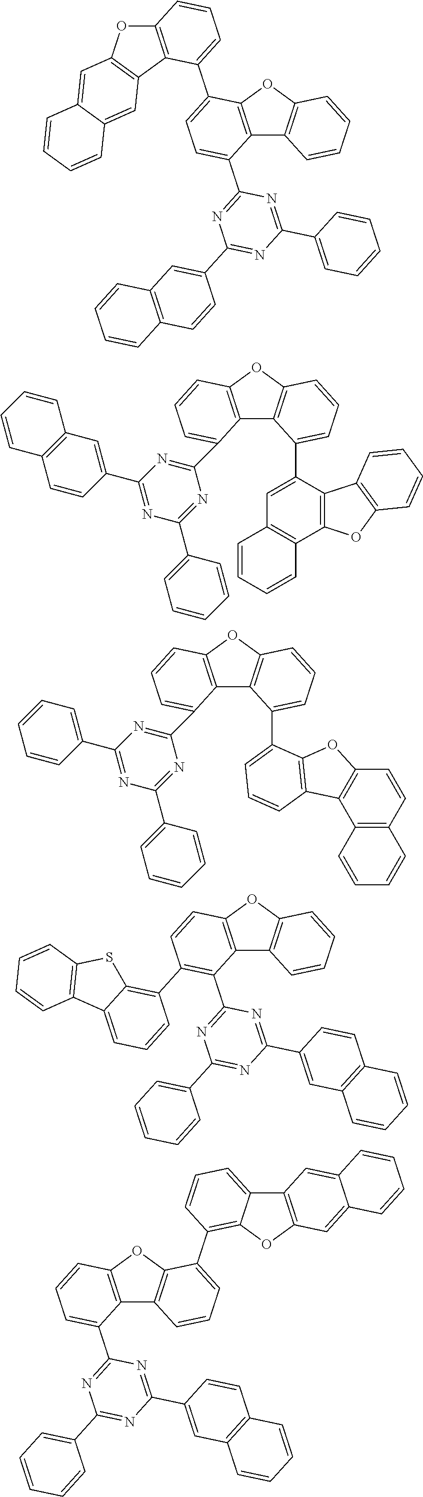

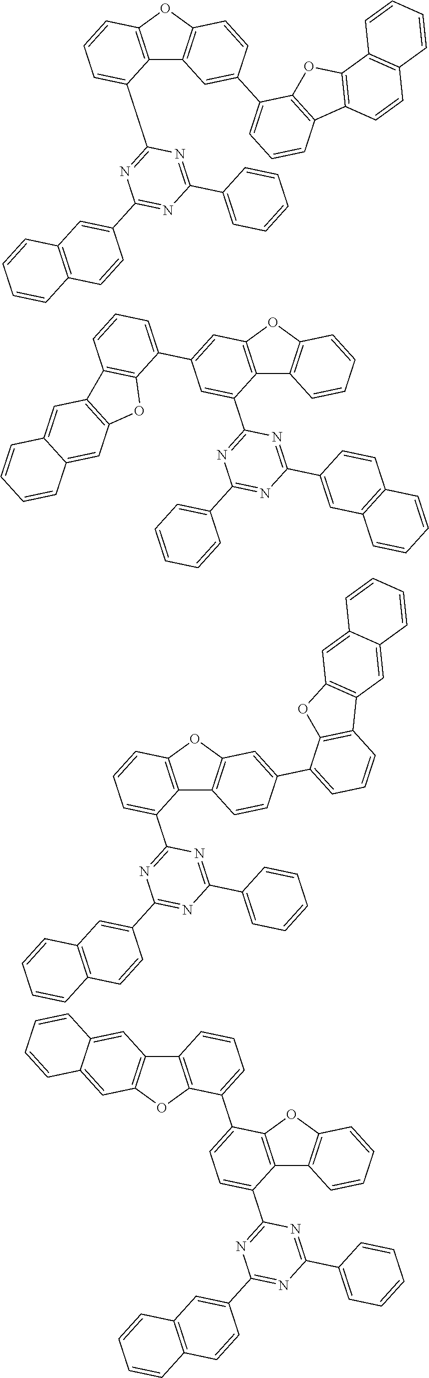

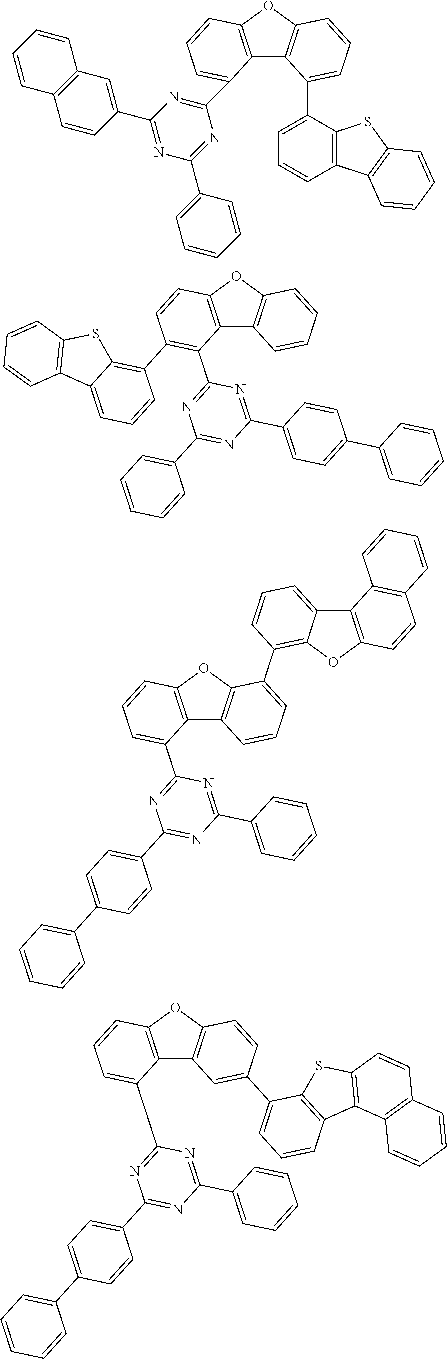

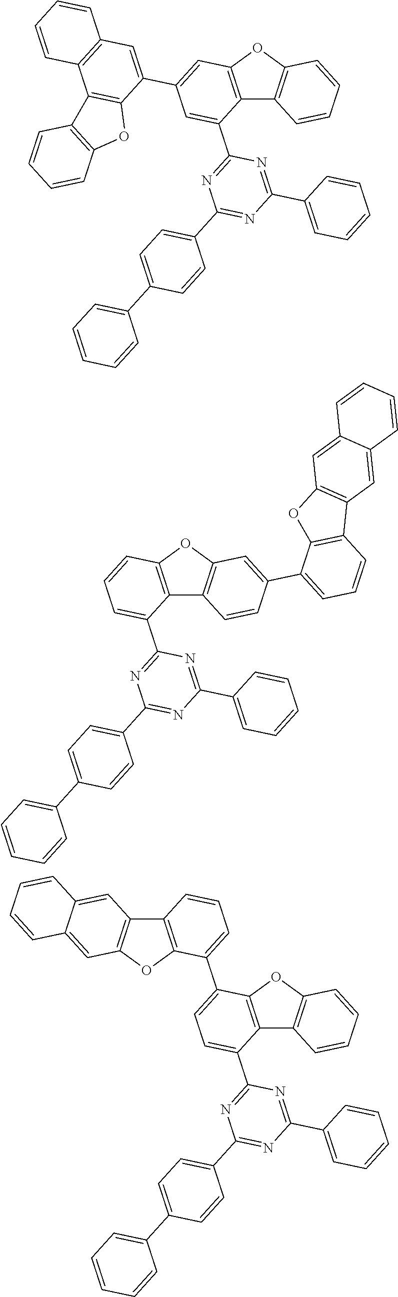

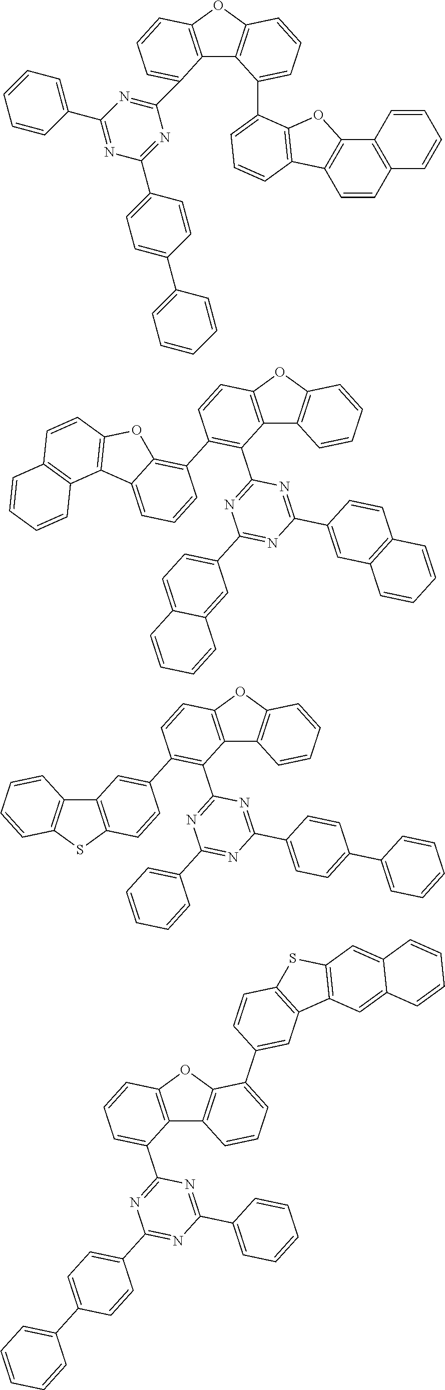

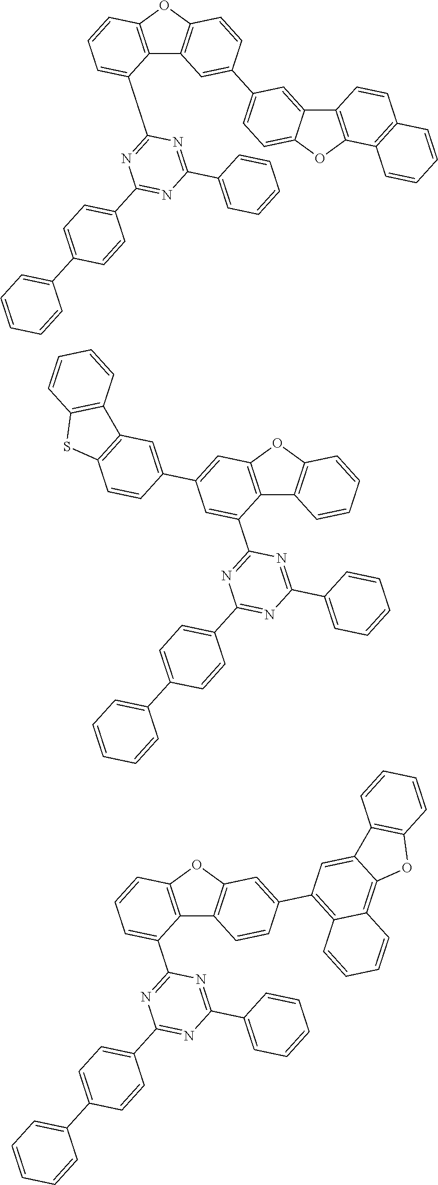

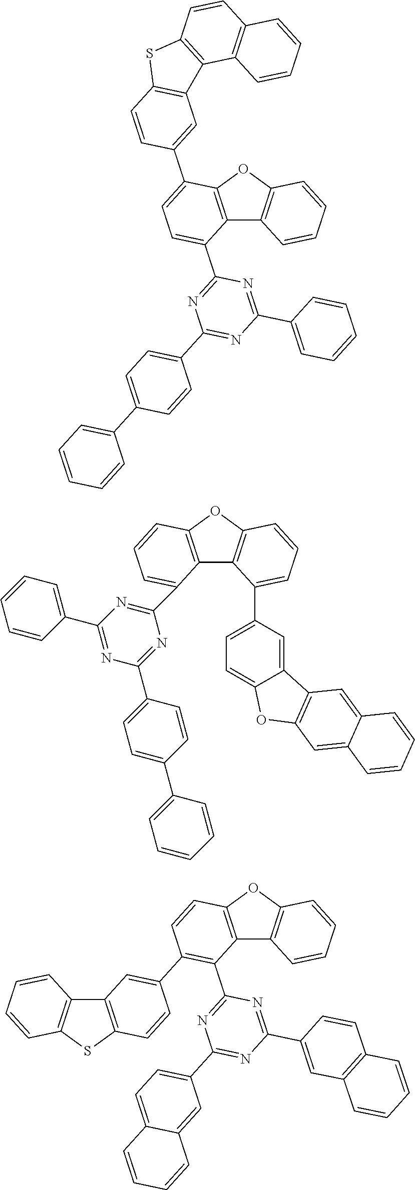

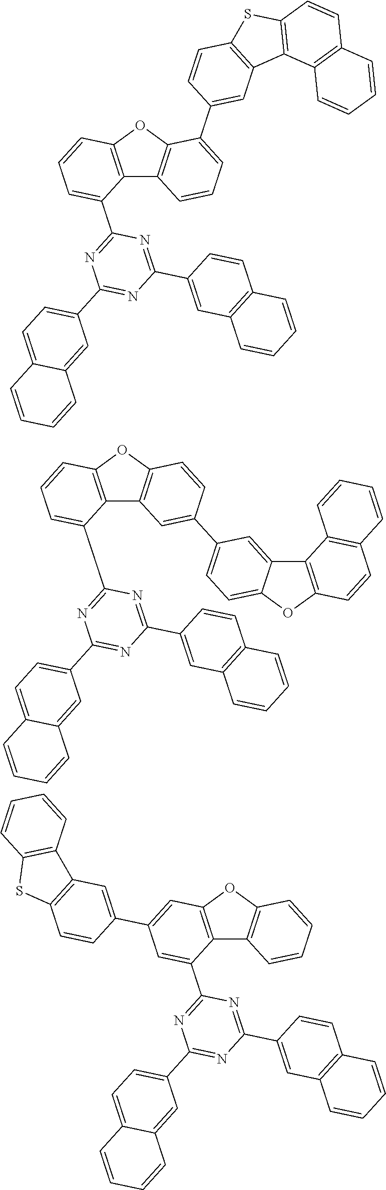

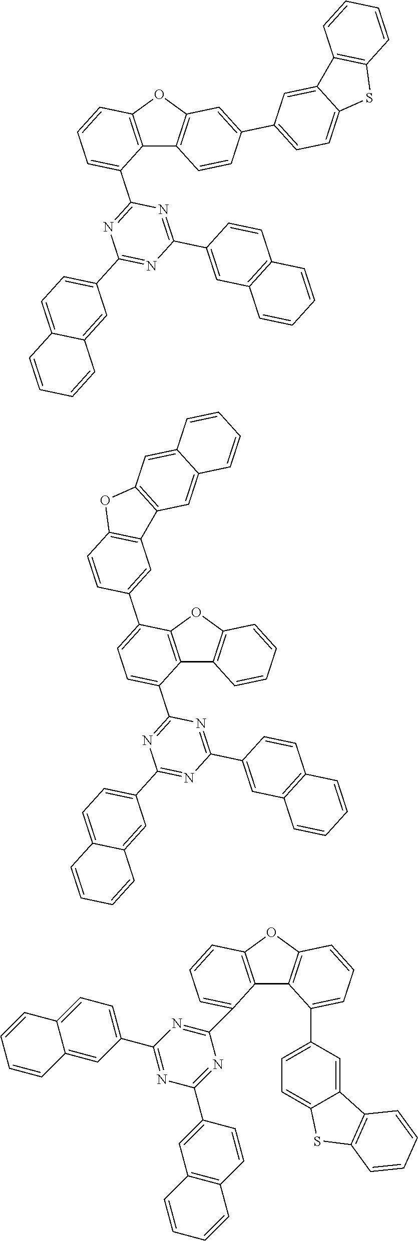 [SMILES: c1ccc2cc(-c3nc(-c4ccc5ccccc5c4)nc(-c4cccc5oc6cc(-c7ccc8sc9ccccc9c8c7)ccc6c45)n3)ccc2c1 ; c1ccc2cc(-c3nc(-c4ccc5ccccc5c4)nc(-c4ccc(-c5ccc6oc7cc8ccccc8cc7c6c5)c5oc6ccccc6c45)n3)ccc2c1 ; c1ccc2cc(-c3nc(-c4ccc5ccccc5c4)nc(-c4cccc5oc6cccc(-c7ccc8sc9ccccc9c8c7)c6c45)n3)ccc2c1]

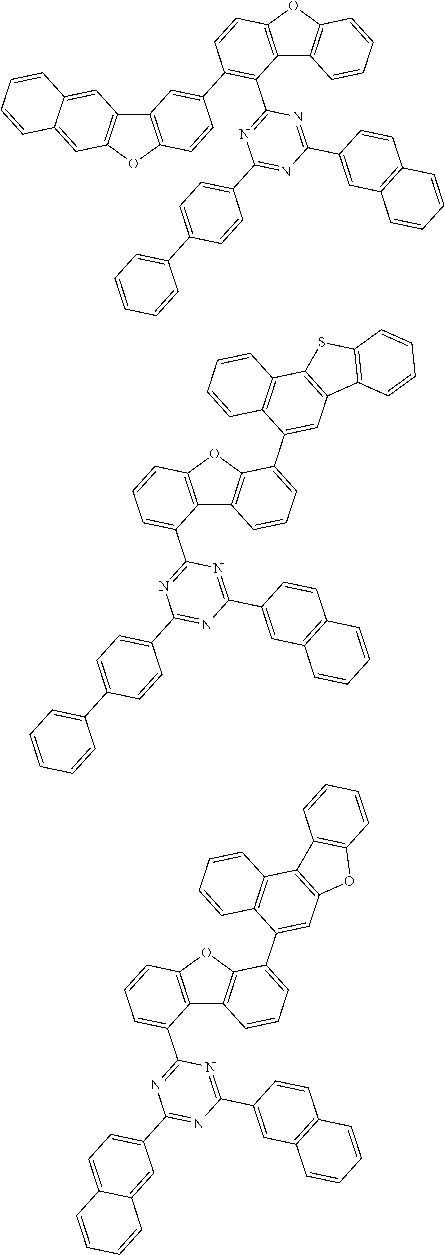 [SMILES: c1ccc(-c2ccc(-c3nc(-c4ccc5ccccc5c4)nc(-c4c(-c5ccc6oc7cc8ccccc8cc7c6c5)ccc5oc6ccccc6c45)n3)cc2)cc1 ; c1ccc(-c2ccc(-c3nc(-c4ccc5ccccc5c4)nc(-c4cccc5oc6c(-c7cc8c9ccccc9sc8c8ccccc78)cccc6c45)n3)cc2)cc1 ; c1ccc2cc(-c3nc(-c4ccc5ccccc5c4)nc(-c4cccc5oc6c(-c7cc8oc9ccccc9c8c8ccccc78)cccc6c45)n3)ccc2c1]

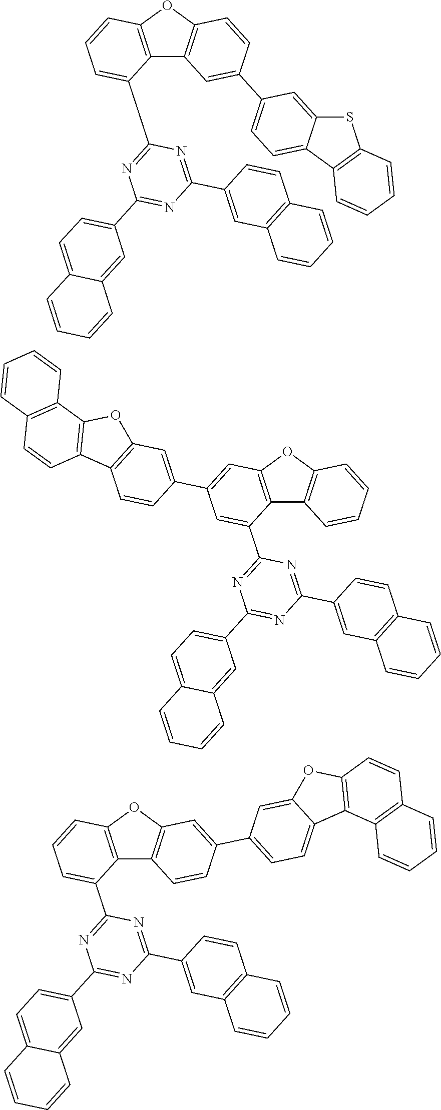

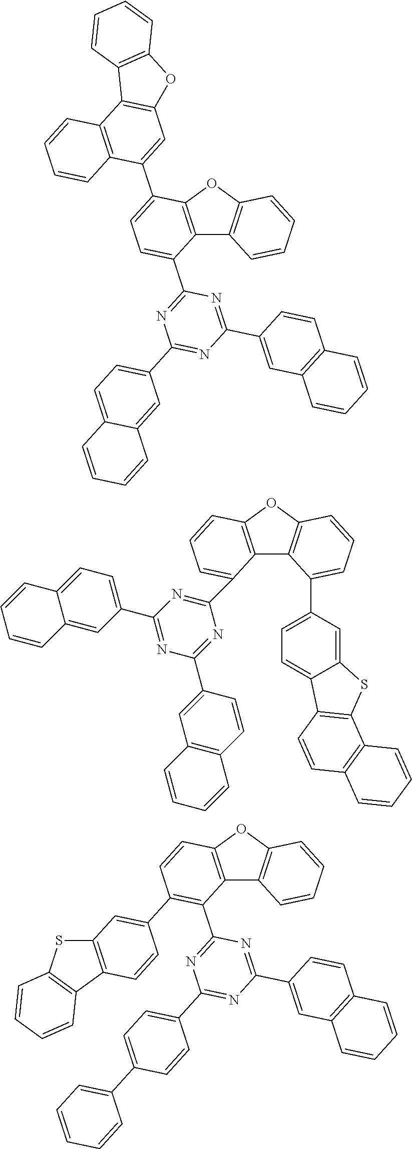

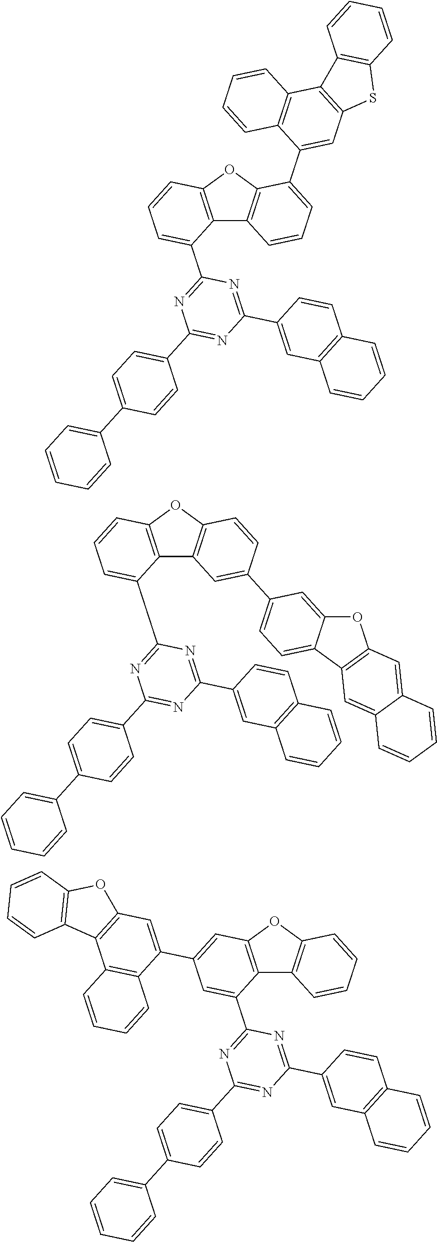

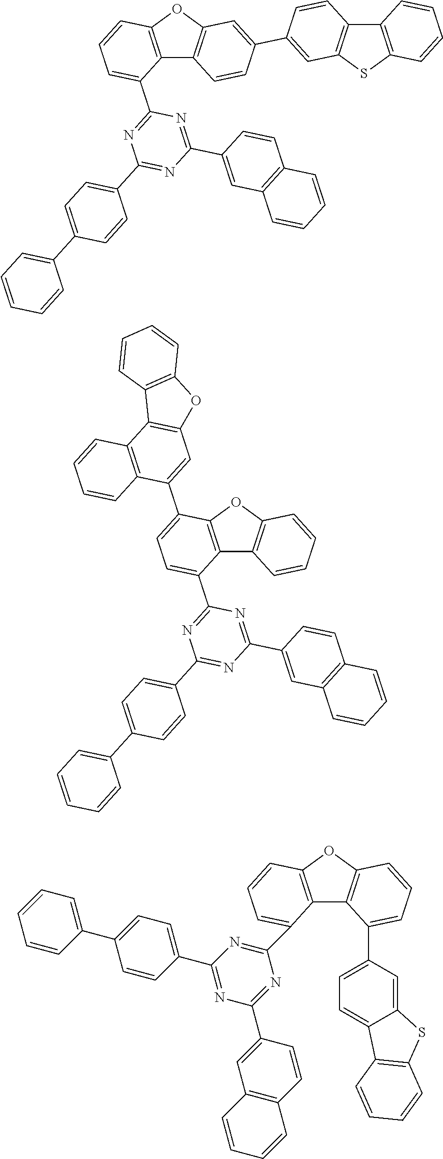 [SMILES: c1ccc(-c2ccc(-c3nc(-c4ccc5ccccc5c4)nc(-c4cccc5oc6cc(-c7ccc8c(c7)sc7ccccc78)ccc6c45)n3)cc2)cc1 ; c1ccc(-c2ccc(-c3nc(-c4ccc5ccccc5c4)nc(-c4ccc(-c5cc6oc7ccccc7c6c6ccccc56)c5oc6ccccc6c45)n3)cc2)cc1 ; c1ccc(-c2ccc(-c3nc(-c4ccc5ccccc5c4)nc(-c4cccc5oc6cccc(-c7ccc8c(c7)sc7ccccc78)c6c45)n3)cc2)cc1]

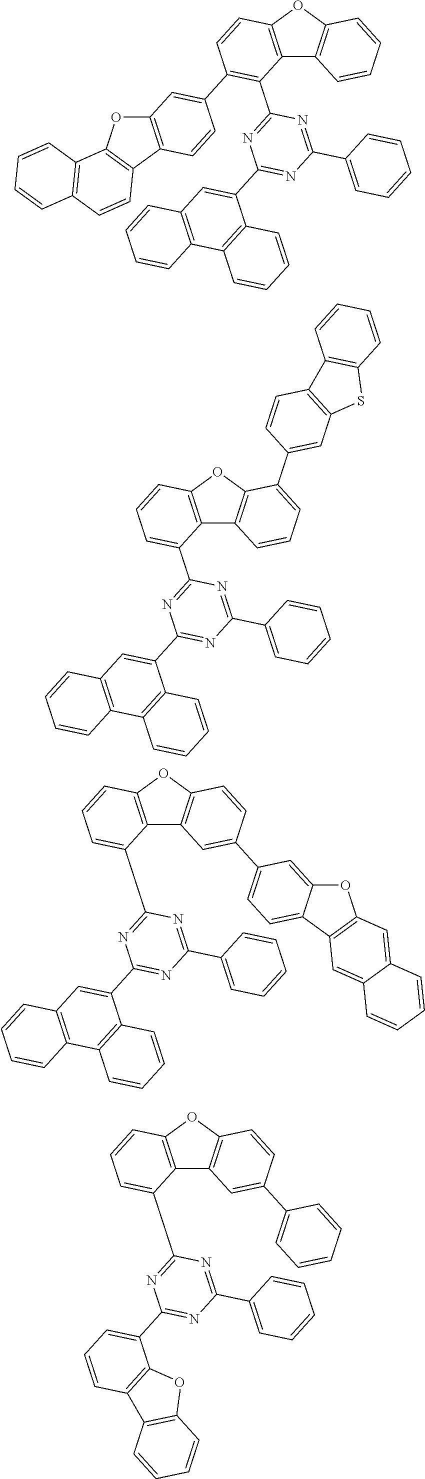

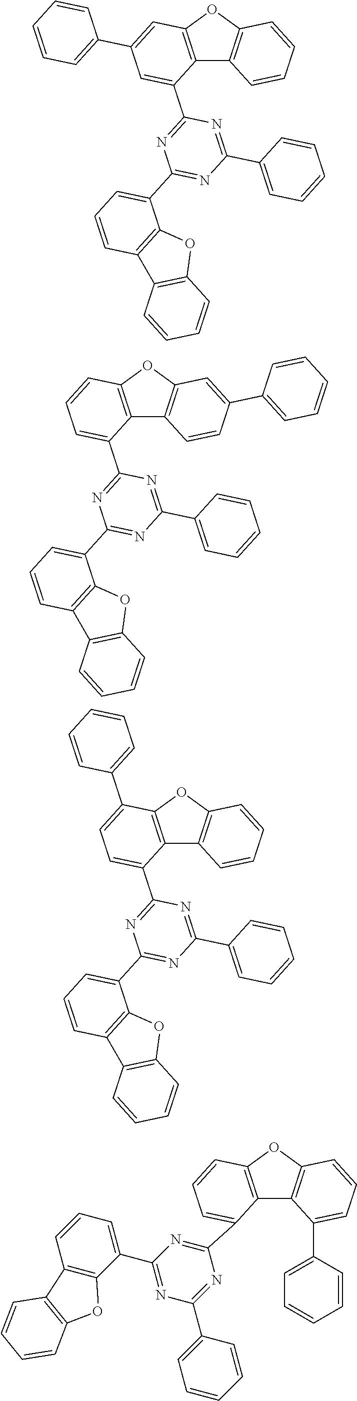

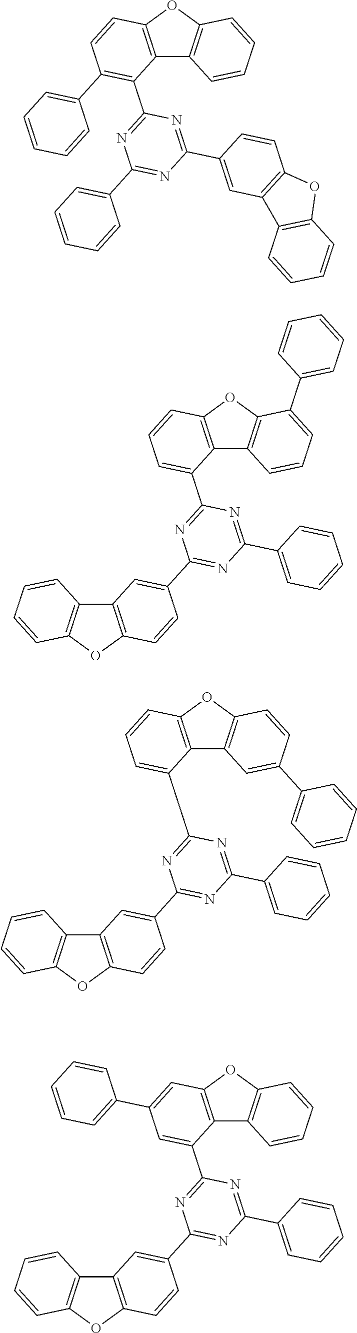

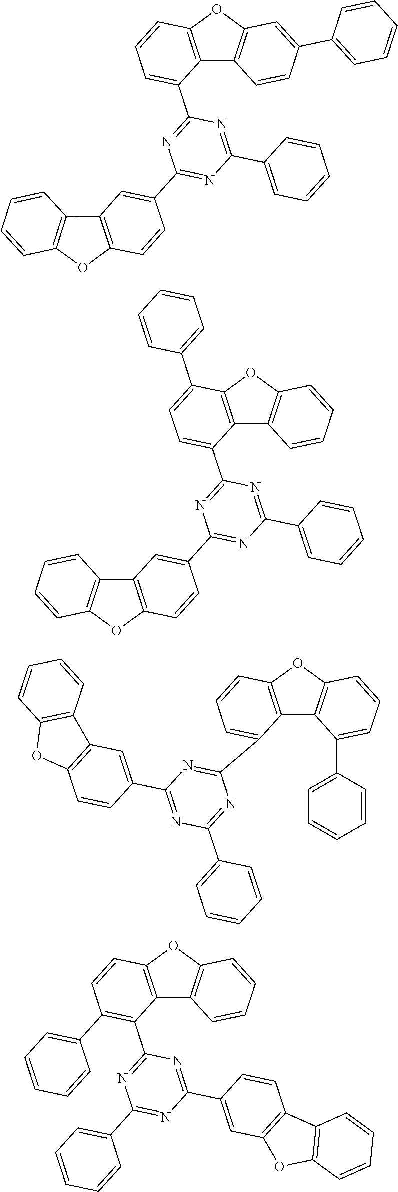

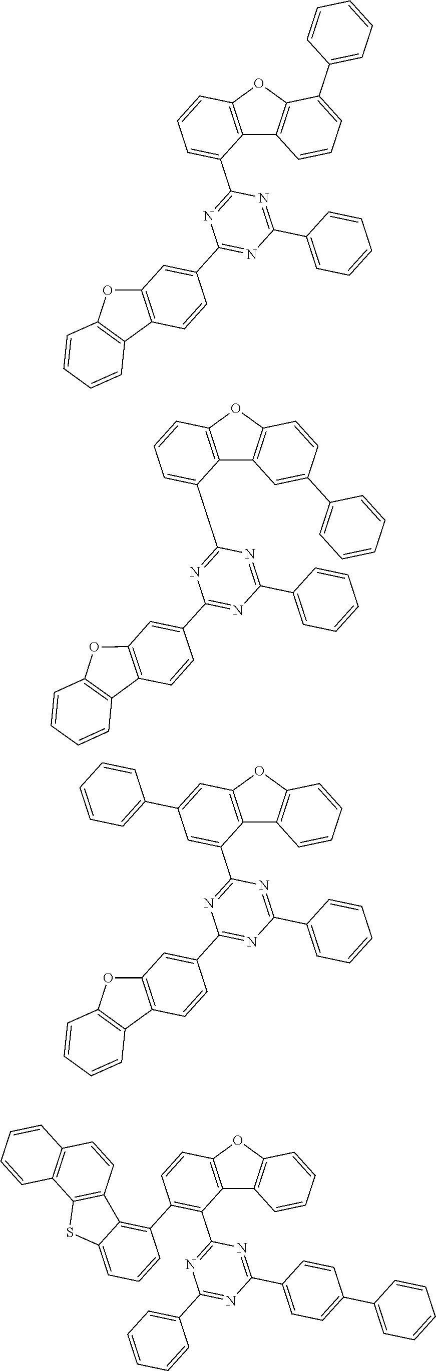

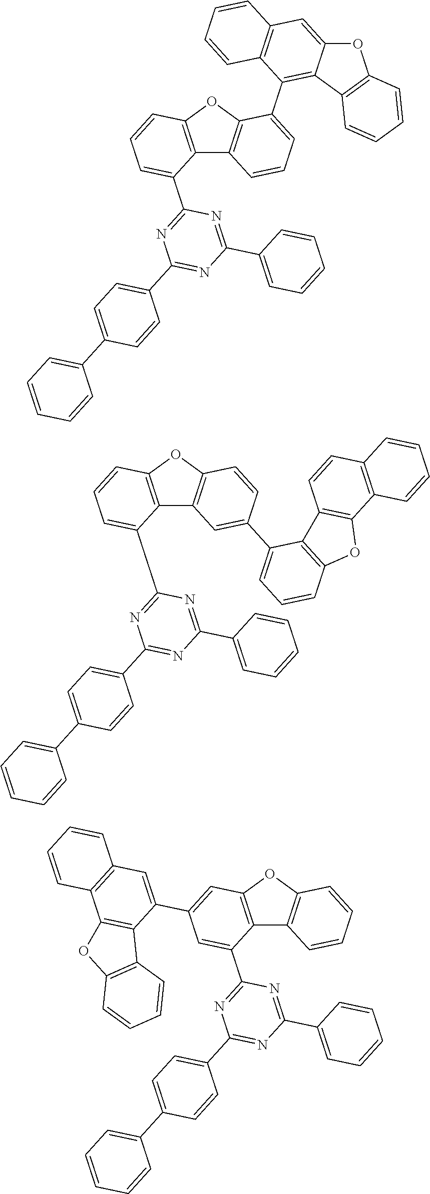

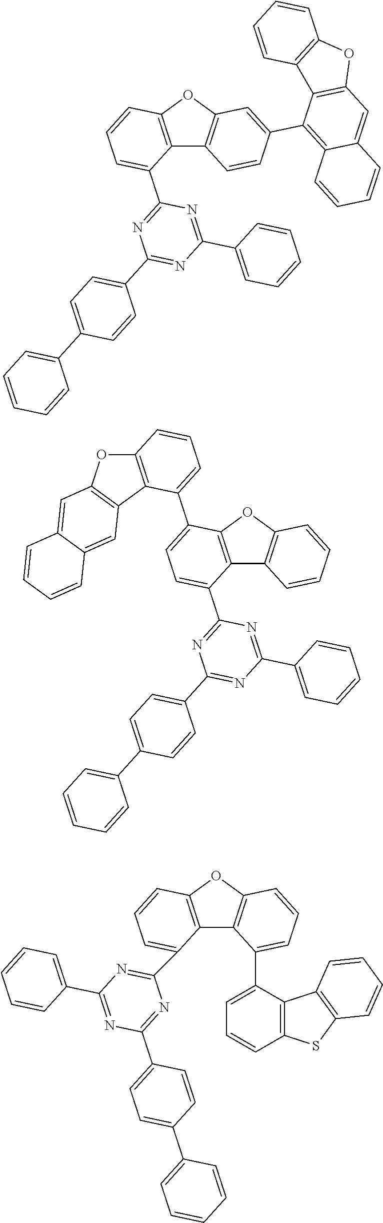 [SMILES: c1ccc(-c2ccc(-c3nc(-c4ccccc4)nc(-c4cccc5oc6cc(-c7c8ccccc8cc8oc9ccccc9c78)ccc6c45)n3)cc2)cc1 ; c1ccc(-c2ccc(-c3nc(-c4ccccc4)nc(-c4ccc(-c5cccc6oc7cc8ccccc8cc7c56)c5oc6ccccc6c45)n3)cc2)cc1 ; c1ccc(-c2ccc(-c3nc(-c4ccccc4)nc(-c4cccc5oc6cccc(-c7cccc8sc9ccccc9c78)c6c45)n3)cc2)cc1]

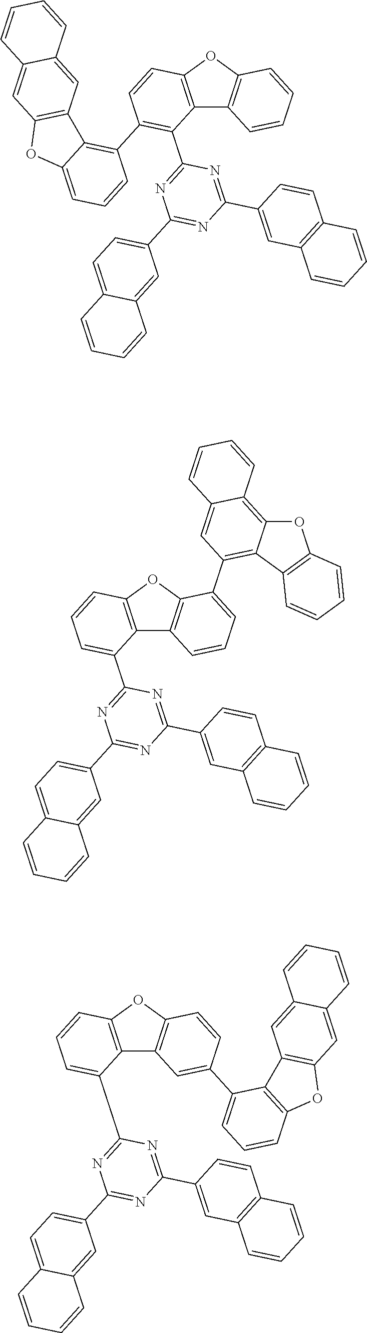 [SMILES: c1ccc2cc(-c3nc(-c4ccc5ccccc5c4)nc(-c4c(-c5cccc6oc7cc8ccccc8cc7c56)ccc5oc6ccccc6c45)n3)ccc2c1 ; c1ccc2cc(-c3nc(-c4ccc5ccccc5c4)nc(-c4cccc5oc6c(-c7cc8ccccc8c8oc9ccccc9c78)cccc6c45)n3)ccc2c1 ; c1ccc2cc(-c3nc(-c4ccc5ccccc5c4)nc(-c4cccc5oc6ccc(-c7cccc8oc9cc%10ccccc%10cc9c78)cc6c45)n3)ccc2c1]

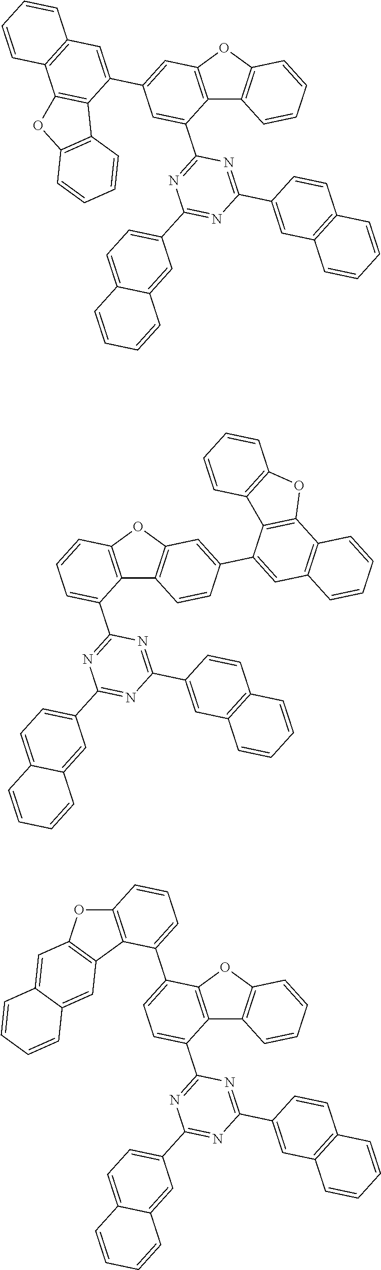 [SMILES: c1ccc2cc(-c3nc(-c4ccc5ccccc5c4)nc(-c4cc(-c5cc6ccccc6c6oc7ccccc7c56)cc5oc6ccccc6c45)n3)ccc2c1 ; c1ccc2cc(-c3nc(-c4ccc5ccccc5c4)nc(-c4cccc5oc6cc(-c7cc8ccccc8c8oc9ccccc9c78)ccc6c45)n3)ccc2c1 ; c1ccc2cc(-c3nc(-c4ccc5ccccc5c4)nc(-c4ccc(-c5cccc6oc7cc8ccccc8cc7c56)c5oc6ccccc6c45)n3)ccc2c1]

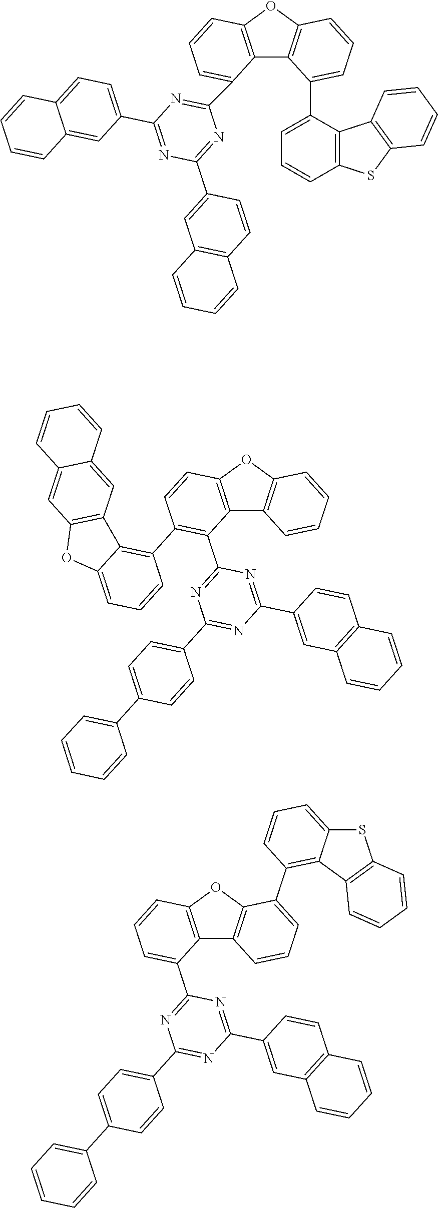 [SMILES: c1ccc2cc(-c3nc(-c4ccc5ccccc5c4)nc(-c4cccc5oc6cccc(-c7cccc8sc9ccccc9c78)c6c45)n3)ccc2c1 ; c1ccc(-c2ccc(-c3nc(-c4ccc5ccccc5c4)nc(-c4c(-c5cccc6oc7cc8ccccc8cc7c56)ccc5oc6ccccc6c45)n3)cc2)cc1 ; c1ccc(-c2ccc(-c3nc(-c4ccc5ccccc5c4)nc(-c4cccc5oc6c(-c7cccc8sc9ccccc9c78)cccc6c45)n3)cc2)cc1]

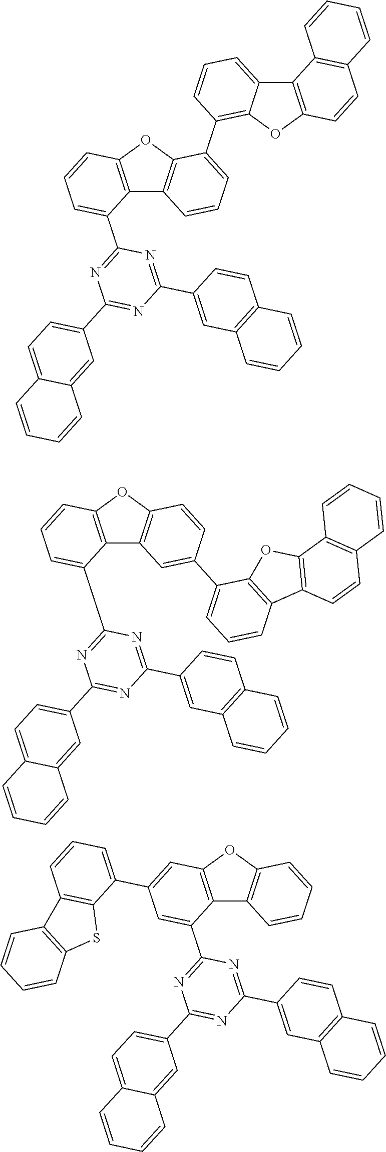

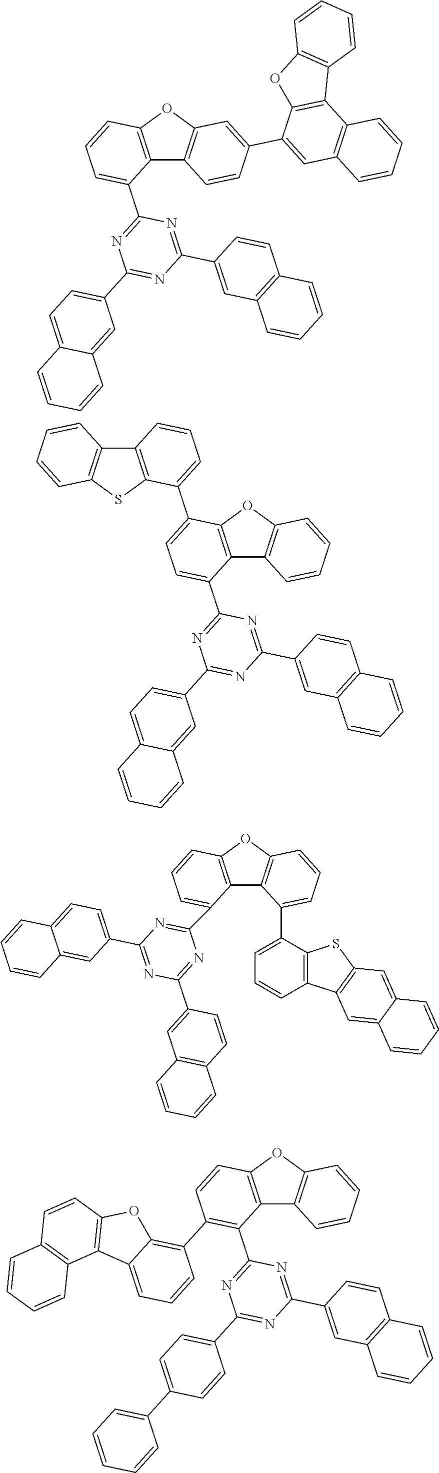

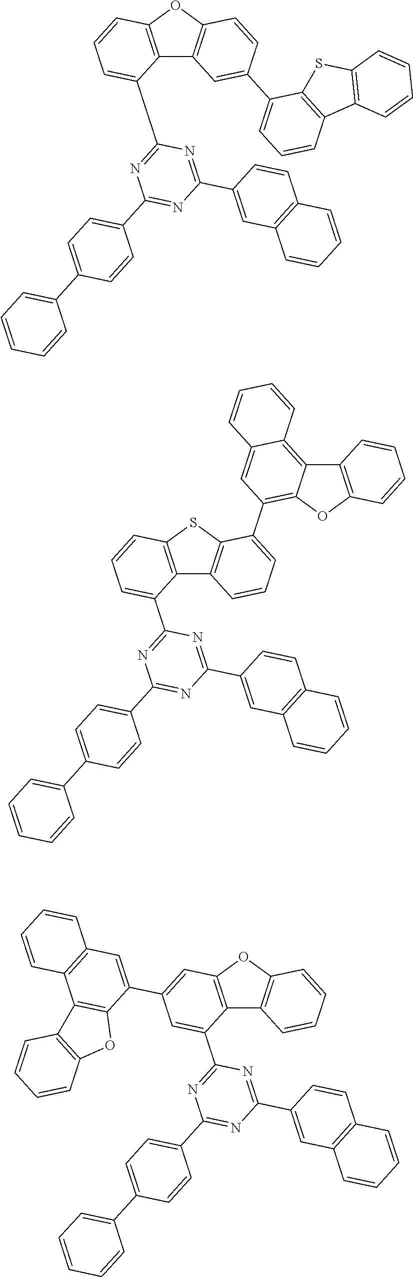 [SMILES: c1ccc(-c2ccc(-c3nc(-c4ccc5ccccc5c4)nc(-c4cccc5oc6ccc(-c7cccc8c7sc7ccccc78)cc6c45)n3)cc2)cc1 ; c1ccc(-c2ccc(-c3nc(-c4ccc5ccccc5c4)nc(-c4cccc5sc6c(-c7cc8ccccc8c8c7oc7ccccc78)cccc6c45)n3)cc2)cc1 ; c1ccc(-c2ccc(-c3nc(-c4ccc5ccccc5c4)nc(-c4cc(-c5cc6ccccc6c6c5oc5ccccc56)cc5oc6ccccc6c45)n3)cc2)cc1]

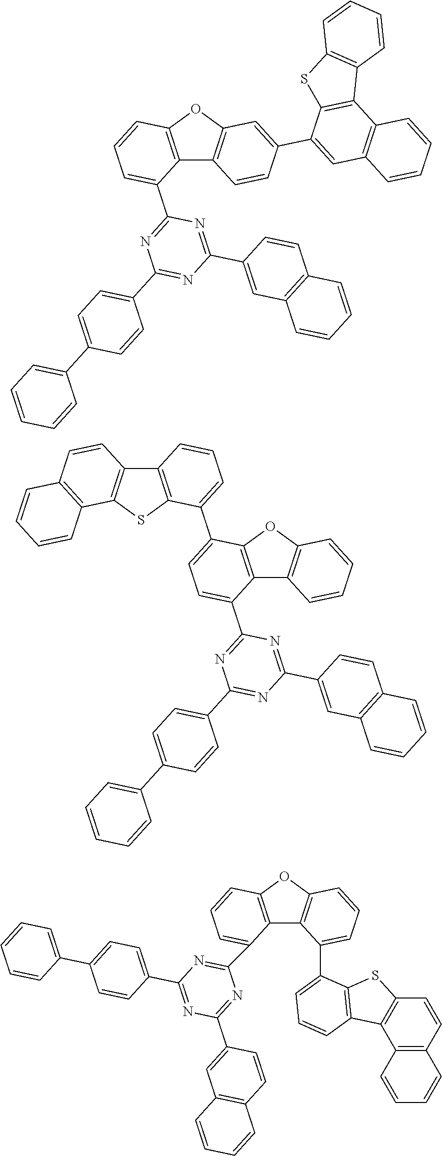

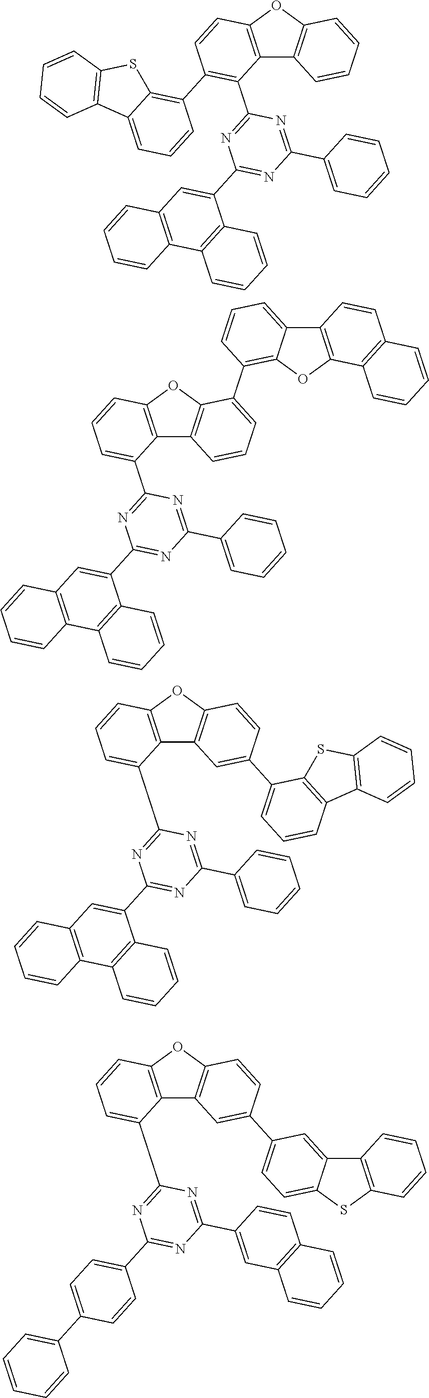

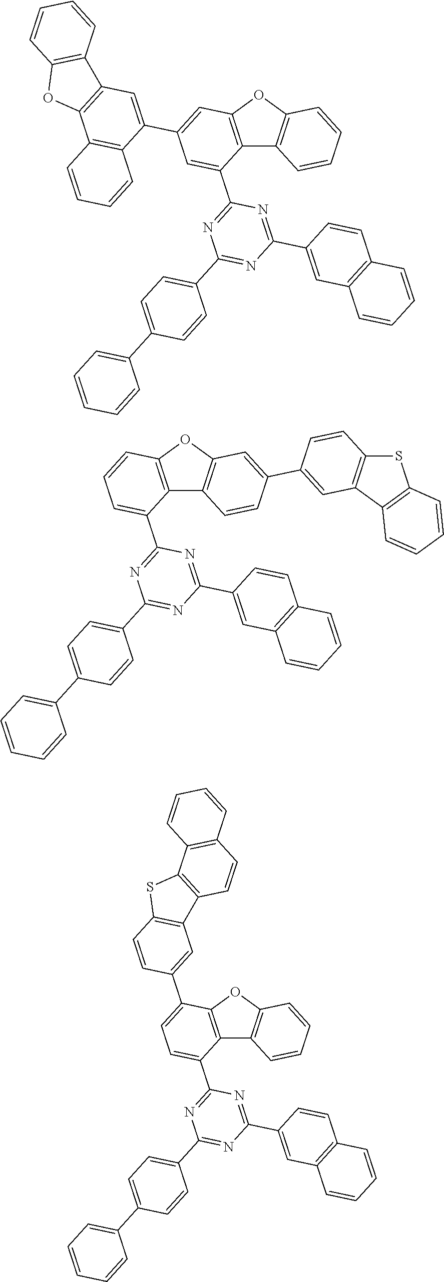

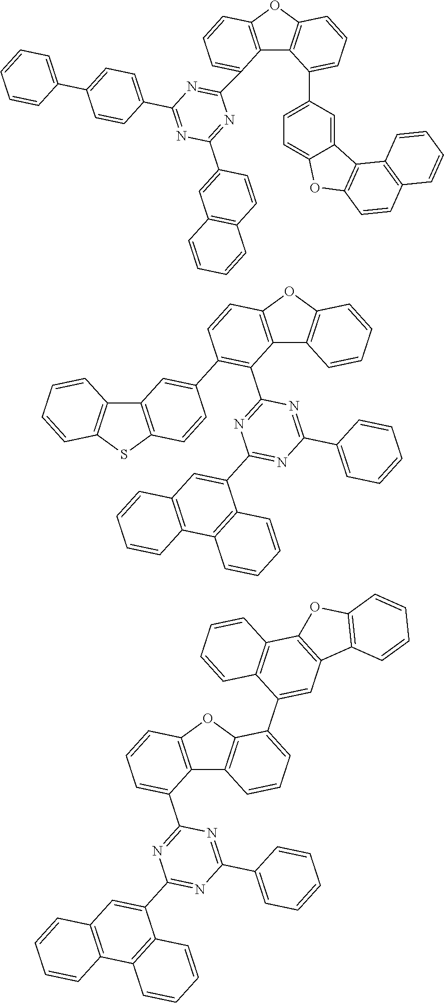

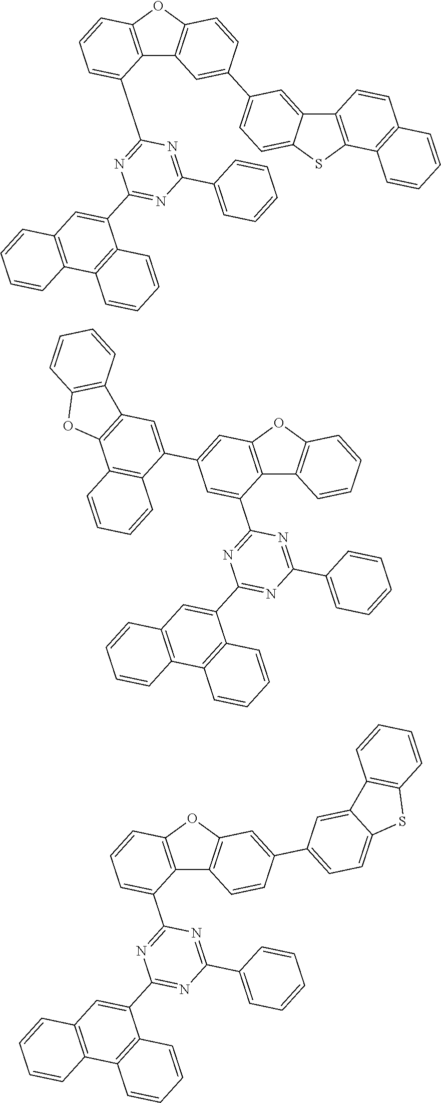

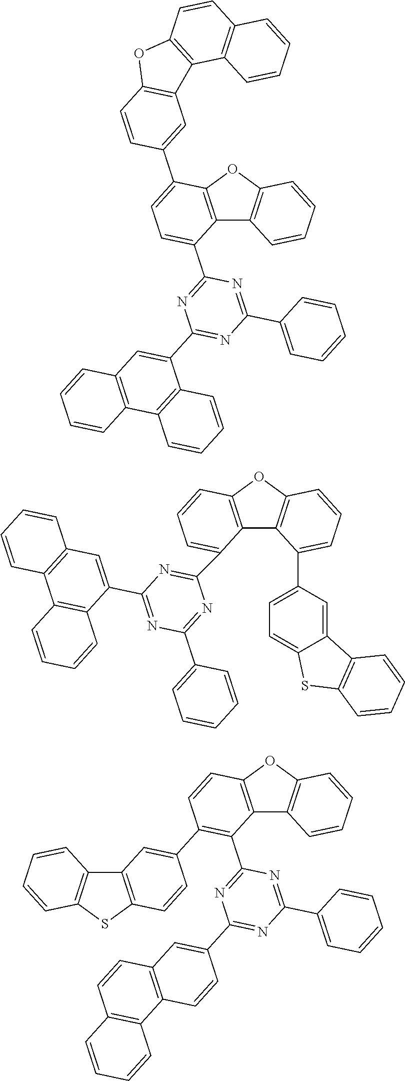

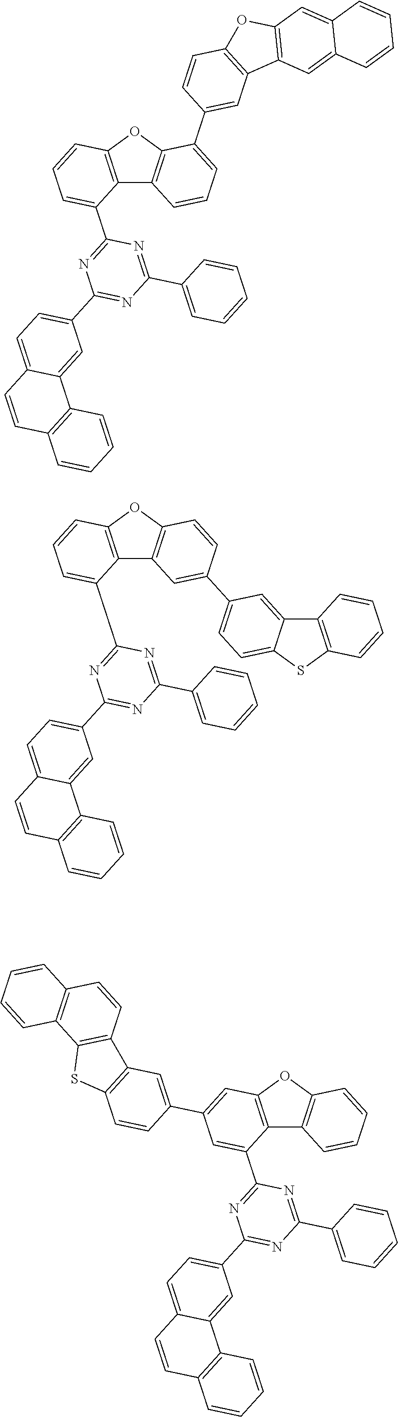 [SMILES: c1ccc(-c2nc(-c3ccc4ccc5ccccc5c4c3)nc(-c3cccc4oc5c(-c6ccc7oc8cc9ccccc9cc8c7c6)cccc5c34)n2)cc1 ; c1ccc(-c2nc(-c3ccc4ccc5ccccc5c4c3)nc(-c3cccc4oc5ccc(-c6ccc7sc8ccccc8c7c6)cc5c34)n2)cc1 ; c1ccc(-c2nc(-c3ccc4ccc5ccccc5c4c3)nc(-c3cc(-c4ccc5sc6c7ccccc7ccc6c5c4)cc4oc5ccccc5c34)n2)cc1]

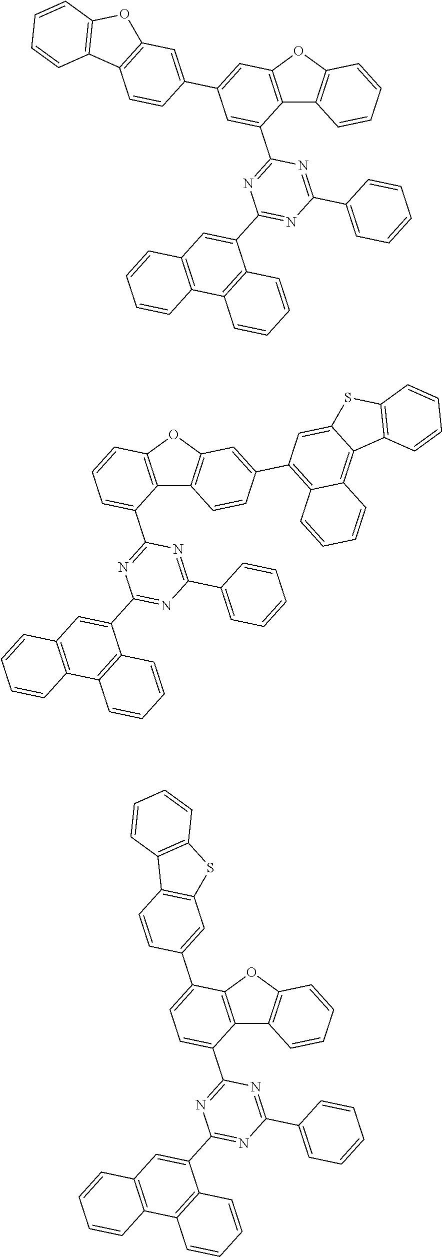 [SMILES: c1ccc(-c2nc(-c3cc4ccccc4c4ccccc34)nc(-c3cc(-c4ccc5c(c4)oc4ccccc45)cc4oc5ccccc5c34)n2)cc1 ; c1ccc(-c2nc(-c3cc4ccccc4c4ccccc34)nc(-c3cccc4oc5cc(-c6cc7sc8ccccc8c7c7ccccc67)ccc5c34)n2)cc1 ; c1ccc(-c2nc(-c3cc4ccccc4c4ccccc34)nc(-c3ccc(-c4ccc5c(c4)sc4ccccc45)c4oc5ccccc5c34)n2)cc1]

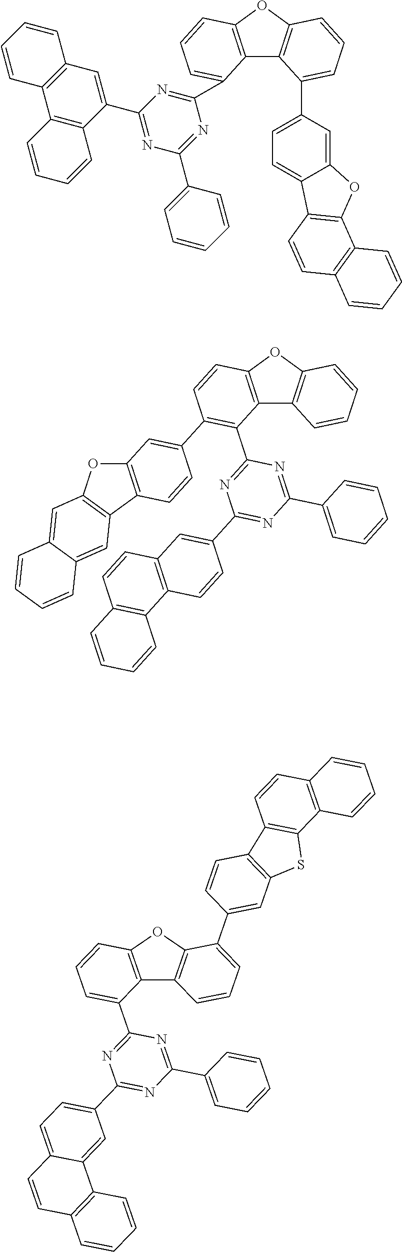 [SMILES: c1ccc(-c2nc(-c3cc4ccccc4c4ccccc34)nc(-c3cccc4oc5cccc(-c6ccc7c(c6)oc6c8ccccc8ccc76)c5c34)n2)cc1 ; c1ccc(-c2nc(-c3ccc4c(ccc5ccccc54)c3)nc(-c3c(-c4ccc5c(c4)oc4cc6ccccc6cc45)ccc4oc5ccccc5c34)n2)cc1 ; c1ccc(-c2nc(-c3ccc4ccc5ccccc5c4c3)nc(-c3cccc4oc5c(-c6ccc7c(c6)sc6c8ccccc8ccc76)cccc5c34)n2)cc1]

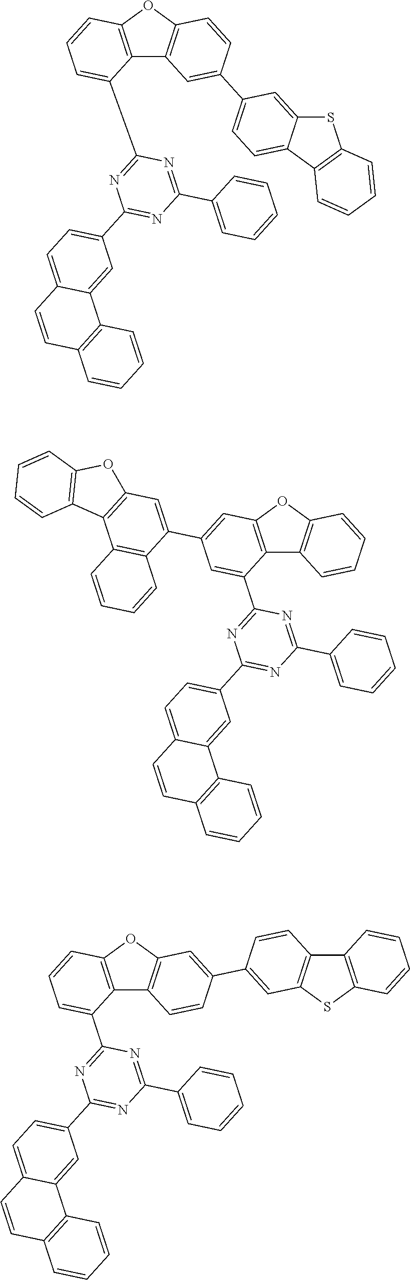 [SMILES: c1ccc(-c2nc(-c3ccc4ccc5ccccc5c4c3)nc(-c3cccc4oc5ccc(-c6ccc7c(c6)sc6ccccc67)cc5c34)n2)cc1 ; c1ccc(-c2nc(-c3ccc4ccc5ccccc5c4c3)nc(-c3cc(-c4cc5oc6ccccc6c5c5ccccc45)cc4oc5ccccc5c34)n2)cc1 ; c1ccc(-c2nc(-c3ccc4ccc5ccccc5c4c3)nc(-c3cccc4oc5cc(-c6ccc7c(c6)sc6ccccc67)ccc5c34)n2)cc1]

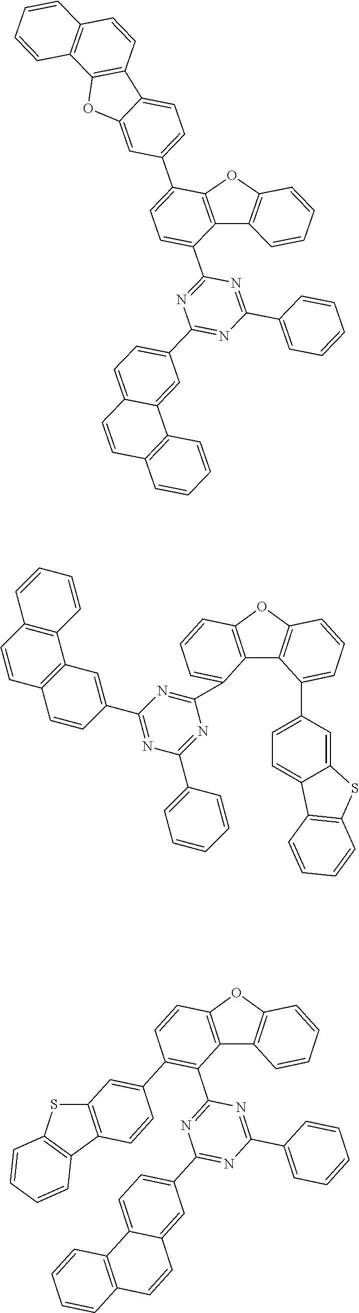 [SMILES: c1ccc(-c2nc(-c3ccc4ccc5ccccc5c4c3)nc(-c3ccc(-c4ccc5c(c4)oc4c6ccccc6ccc54)c4oc5ccccc5c34)n2)cc1 ; c1ccc(-c2nc(-c3ccc4ccc5ccccc5c4c3)nc(-c3cccc4oc5cccc(-c6ccc7c(c6)sc6ccccc67)c5c34)n2)cc1 ; c1ccc(-c2nc(-c3ccc4c(ccc5ccccc54)c3)nc(-c3c(-c4ccc5c(c4)sc4ccccc45)ccc4oc5ccccc5c34)n2)cc1]

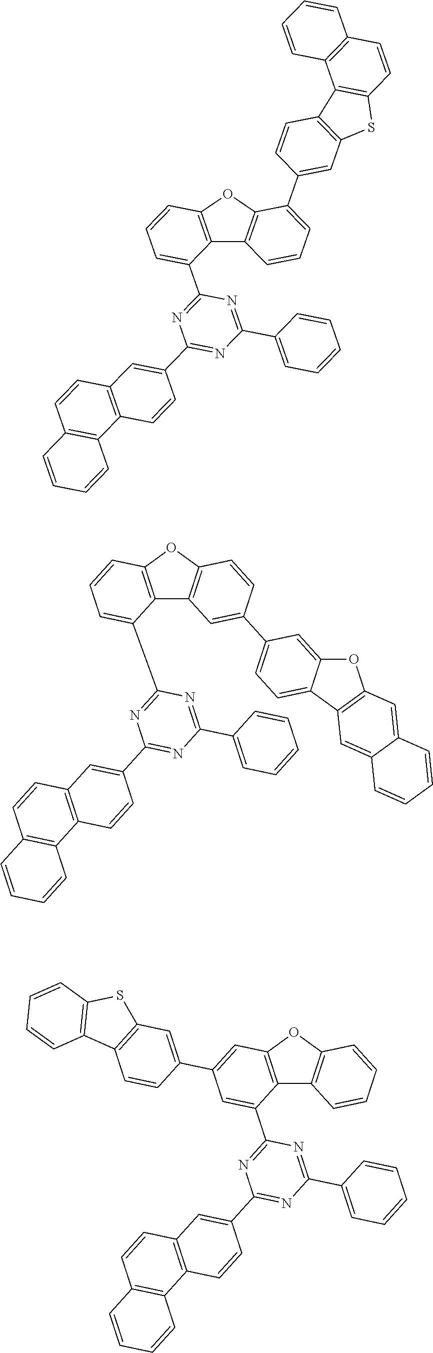 [SMILES: c1ccc(-c2nc(-c3ccc4c(ccc5ccccc54)c3)nc(-c3cccc4oc5c(-c6ccc7c(c6)sc6ccc8ccccc8c67)cccc5c34)n2)cc1 ; c1ccc(-c2nc(-c3ccc4c(ccc5ccccc54)c3)nc(-c3cccc4oc5ccc(-c6ccc7c(c6)oc6cc8ccccc8cc67)cc5c34)n2)cc1 ; c1ccc(-c2nc(-c3ccc4c(ccc5ccccc54)c3)nc(-c3cc(-c4ccc5c(c4)sc4ccccc45)cc4oc5ccccc5c34)n2)cc1]

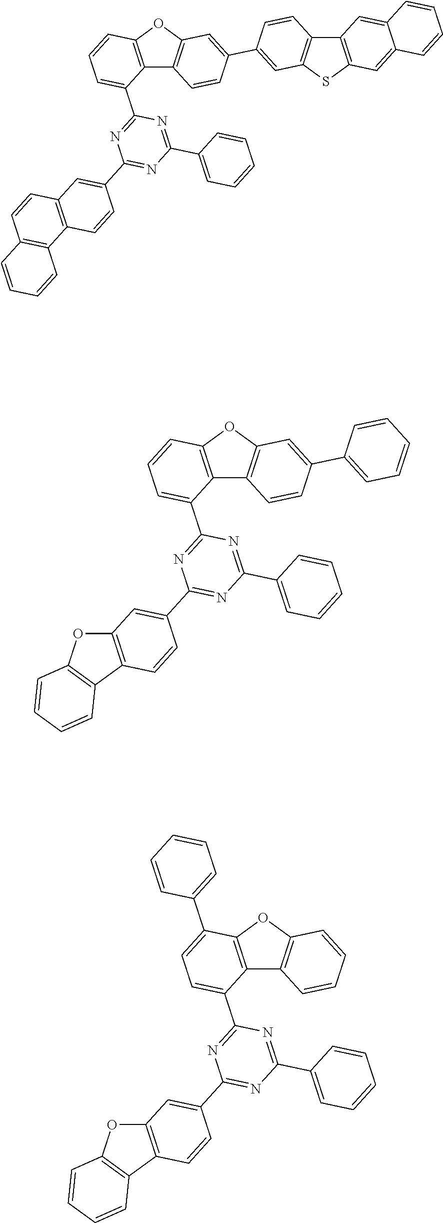 [SMILES: c1ccc(-c2nc(-c3ccc4c(ccc5ccccc54)c3)nc(-c3cccc4oc5cc(-c6ccc7c(c6)sc6cc8ccccc8cc67)ccc5c34)n2)cc1 ; c1ccc(-c2ccc3c(c2)oc2cccc(-c4nc(-c5ccccc5)nc(-c5ccc6c(c5)oc5ccccc56)n4)c23)cc1 ; c1ccc(-c2nc(-c3ccc4c(c3)oc3ccccc34)nc(-c3ccc(-c4ccccc4)c4oc5ccccc5c34)n2)cc1]

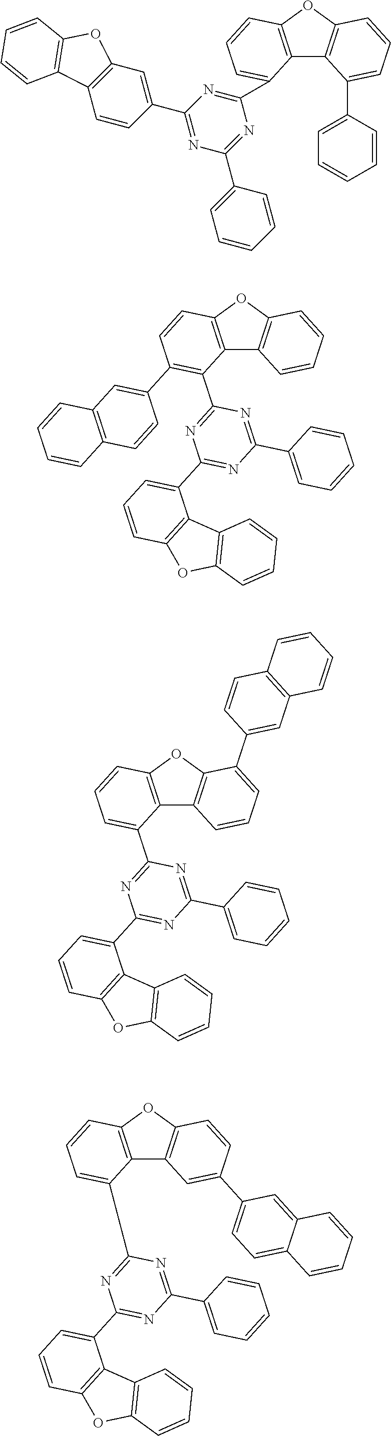 [SMILES: c1ccc(-c2nc(-c3ccc4c(c3)oc3ccccc34)nc(-c3cccc4oc5cccc(-c6ccccc6)c5c34)n2)cc1 ; c1ccc(-c2nc(-c3cccc4oc5ccccc5c34)nc(-c3c(-c4ccc5ccccc5c4)ccc4oc5ccccc5c34)n2)cc1 ; c1ccc(-c2nc(-c3cccc4oc5ccccc5c34)nc(-c3cccc4oc5c(-c6ccc7ccccc7c6)cccc5c34)n2)cc1 ; c1ccc(-c2nc(-c3cccc4oc5ccccc5c34)nc(-c3cccc4oc5ccc(-c6ccc7ccccc7c6)cc5c34)n2)cc1]

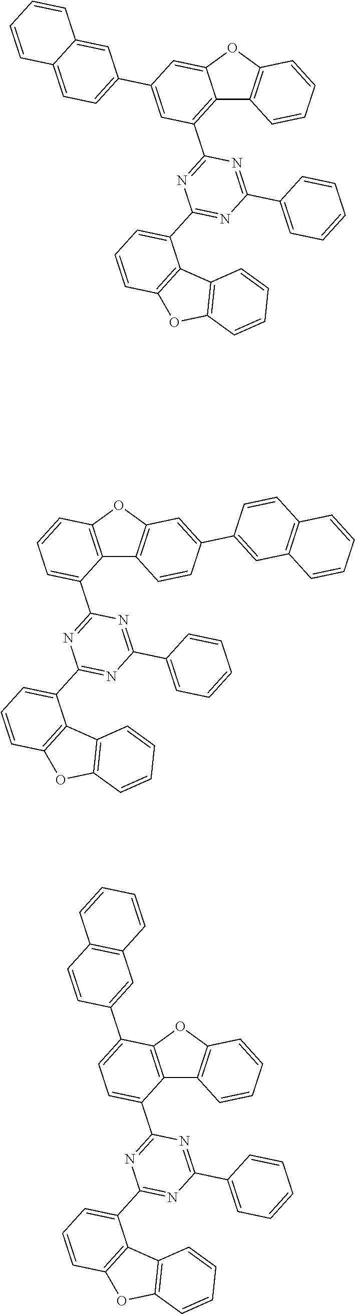 [SMILES: c1ccc(-c2nc(-c3cccc4oc5ccccc5c34)nc(-c3cc(-c4ccc5ccccc5c4)cc4oc5ccccc5c34)n2)cc1 ; c1ccc(-c2nc(-c3cccc4oc5ccccc5c34)nc(-c3cccc4oc5cc(-c6ccc7ccccc7c6)ccc5c34)n2)cc1 ; c1ccc(-c2nc(-c3cccc4oc5ccccc5c34)nc(-c3ccc(-c4ccc5ccccc5c4)c4oc5ccccc5c34)n2)cc1]

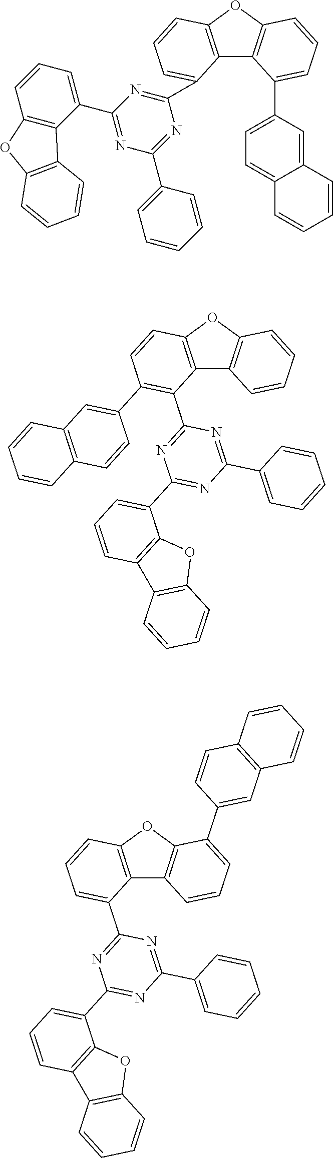 [SMILES: c1ccc(-c2nc(-c3cccc4oc5ccccc5c34)nc(-c3cccc4oc5cccc(-c6ccc7ccccc7c6)c5c34)n2)cc1 ; c1ccc(-c2nc(-c3cccc4c3oc3ccccc34)nc(-c3c(-c4ccc5ccccc5c4)ccc4oc5ccccc5c34)n2)cc1 ; c1ccc(-c2nc(-c3cccc4c3oc3ccccc34)nc(-c3cccc4oc5c(-c6ccc7ccccc7c6)cccc5c34)n2)cc1]

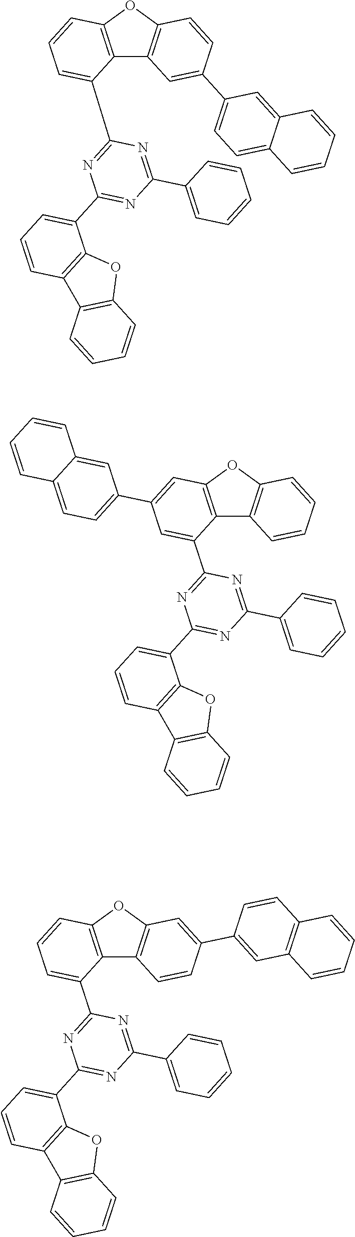 [SMILES: c1ccc(-c2nc(-c3cccc4c3oc3ccccc34)nc(-c3cccc4oc5ccc(-c6ccc7ccccc7c6)cc5c34)n2)cc1 ; c1ccc(-c2nc(-c3cccc4c3oc3ccccc34)nc(-c3cc(-c4ccc5ccccc5c4)cc4oc5ccccc5c34)n2)cc1 ; c1ccc(-c2nc(-c3cccc4c3oc3ccccc34)nc(-c3cccc4oc5cc(-c6ccc7ccccc7c6)ccc5c34)n2)cc1]

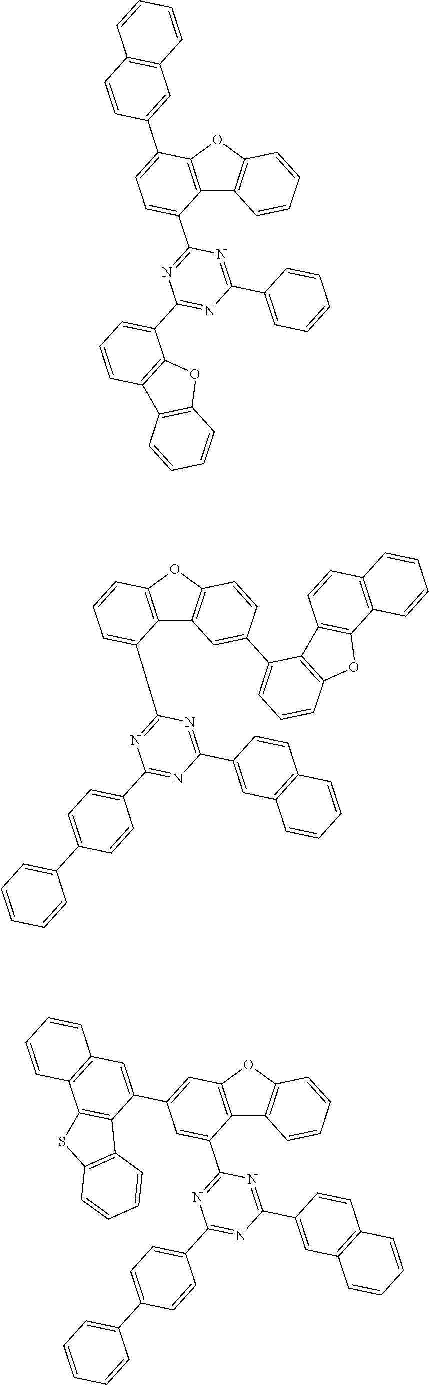 [SMILES: c1ccc(-c2nc(-c3cccc4c3oc3ccccc34)nc(-c3ccc(-c4ccc5ccccc5c4)c4oc5ccccc5c34)n2)cc1 ; c1ccc(-c2ccc(-c3nc(-c4ccc5ccccc5c4)nc(-c4cccc5oc6ccc(-c7cccc8oc9c%10ccccc%10ccc9c78)cc6c45)n3)cc2)cc1 ; c1ccc(-c2ccc(-c3nc(-c4ccc5ccccc5c4)nc(-c4cc(-c5cc6ccccc6c6sc7ccccc7c56)cc5oc6ccccc6c45)n3)cc2)cc1]

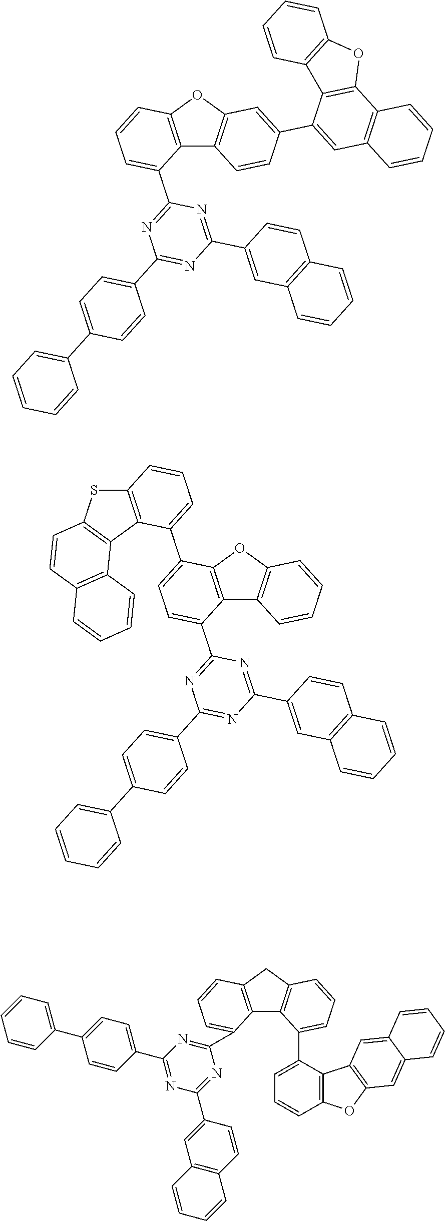 [SMILES: c1ccc(-c2ccc(-c3nc(-c4ccc5ccccc5c4)nc(-c4cccc5oc6cc(-c7cc8ccccc8c8oc9ccccc9c78)ccc6c45)n3)cc2)cc1 ; c1ccc(-c2ccc(-c3nc(-c4ccc5ccccc5c4)nc(-c4ccc(-c5cccc6sc7ccc8ccccc8c7c56)c5oc6ccccc6c45)n3)cc2)cc1 ; c1ccc(-c2ccc(-c3nc(-c4ccc5ccccc5c4)nc(-c4cccc5c4-c4c(cccc4-c4cccc6oc7cc8ccccc8cc7c46)C5)n3)cc2)cc1]

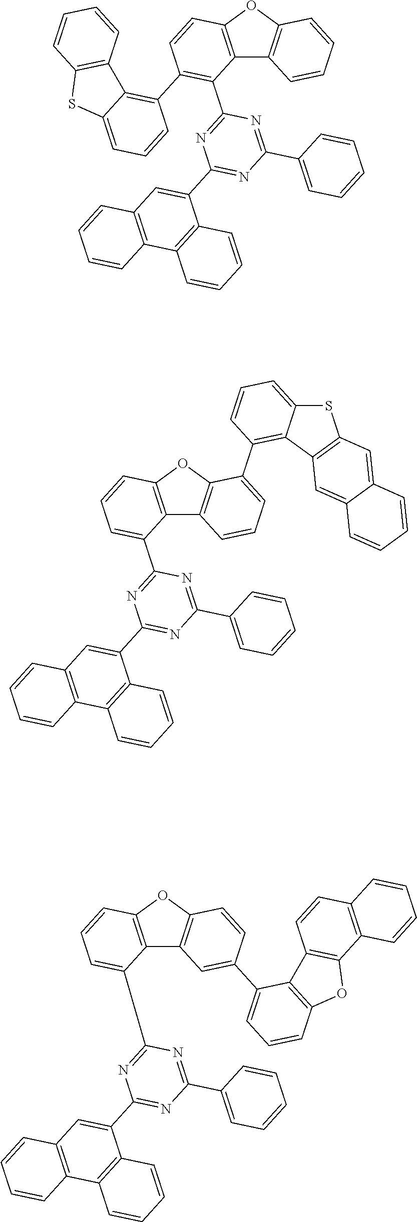 [SMILES: c1ccc(-c2nc(-c3cc4ccccc4c4ccccc34)nc(-c3c(-c4cccc5sc6ccccc6c45)ccc4oc5ccccc5c34)n2)cc1 ; c1ccc(-c2nc(-c3cc4ccccc4c4ccccc34)nc(-c3cccc4oc5c(-c6cccc7sc8cc9ccccc9cc8c67)cccc5c34)n2)cc1 ; c1ccc(-c2nc(-c3cc4ccccc4c4ccccc34)nc(-c3cccc4oc5ccc(-c6cccc7oc8c9ccccc9ccc8c67)cc5c34)n2)cc1]

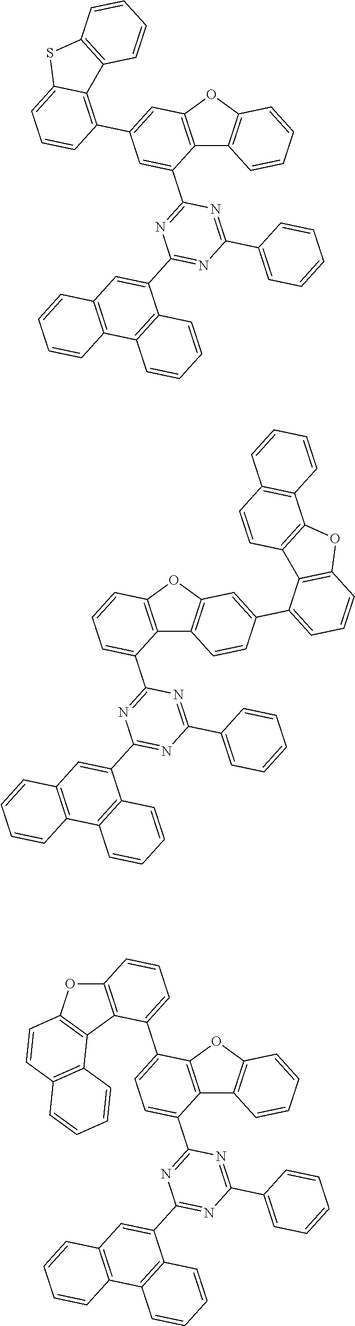 [SMILES: c1ccc(-c2nc(-c3cc4ccccc4c4ccccc34)nc(-c3cc(-c4cccc5sc6ccccc6c45)cc4oc5ccccc5c34)n2)cc1 ; c1ccc(-c2nc(-c3cc4ccccc4c4ccccc34)nc(-c3cccc4oc5cc(-c6cccc7oc8c9ccccc9ccc8c67)ccc5c34)n2)cc1 ; c1ccc(-c2nc(-c3cc4ccccc4c4ccccc34)nc(-c3ccc(-c4cccc5oc6ccc7ccccc7c6c45)c4oc5ccccc5c34)n2)cc1]

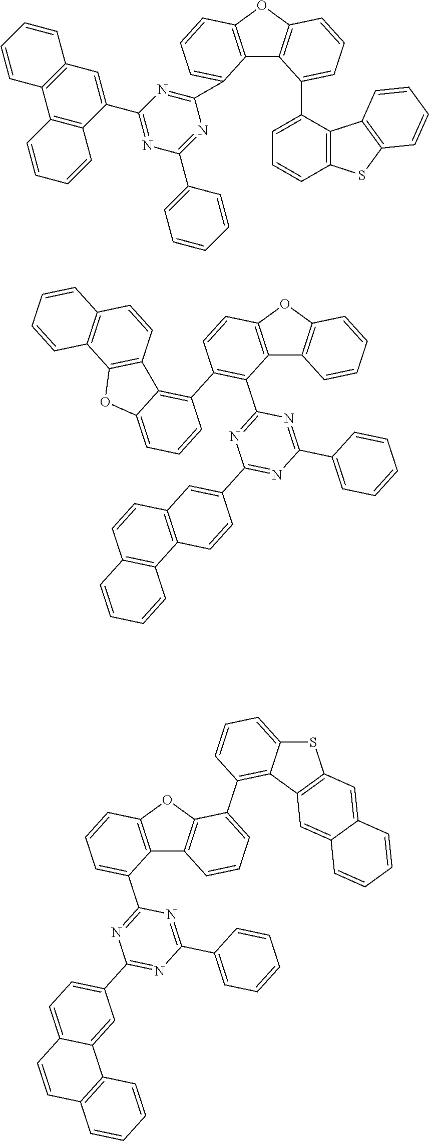 [SMILES: c1ccc(-c2nc(-c3cc4ccccc4c4ccccc34)nc(-c3cccc4oc5cccc(-c6cccc7sc8ccccc8c67)c5c34)n2)cc1 ; c1ccc(-c2nc(-c3ccc4c(ccc5ccccc54)c3)nc(-c3c(-c4cccc5oc6c7ccccc7ccc6c45)ccc4oc5ccccc5c34)n2)cc1 ; c1ccc(-c2nc(-c3ccc4ccc5ccccc5c4c3)nc(-c3cccc4oc5c(-c6cccc7sc8cc9ccccc9cc8c67)cccc5c34)n2)cc1]

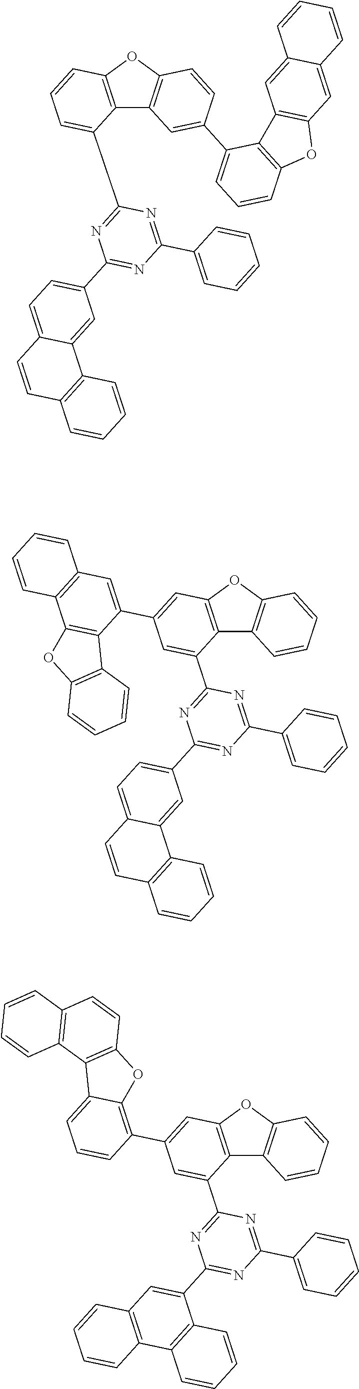 [SMILES: c1ccc(-c2nc(-c3ccc4ccc5ccccc5c4c3)nc(-c3cccc4oc5ccc(-c6cccc7oc8cc9ccccc9cc8c67)cc5c34)n2)cc1 ; c1ccc(-c2nc(-c3ccc4ccc5ccccc5c4c3)nc(-c3cc(-c4cc5ccccc5c5oc6ccccc6c45)cc4oc5ccccc5c34)n2)cc1 ; c1ccc(-c2nc(-c3cc4ccccc4c4ccccc34)nc(-c3cc(-c4cccc5c4oc4ccc6ccccc6c45)cc4oc5ccccc5c34)n2)cc1]

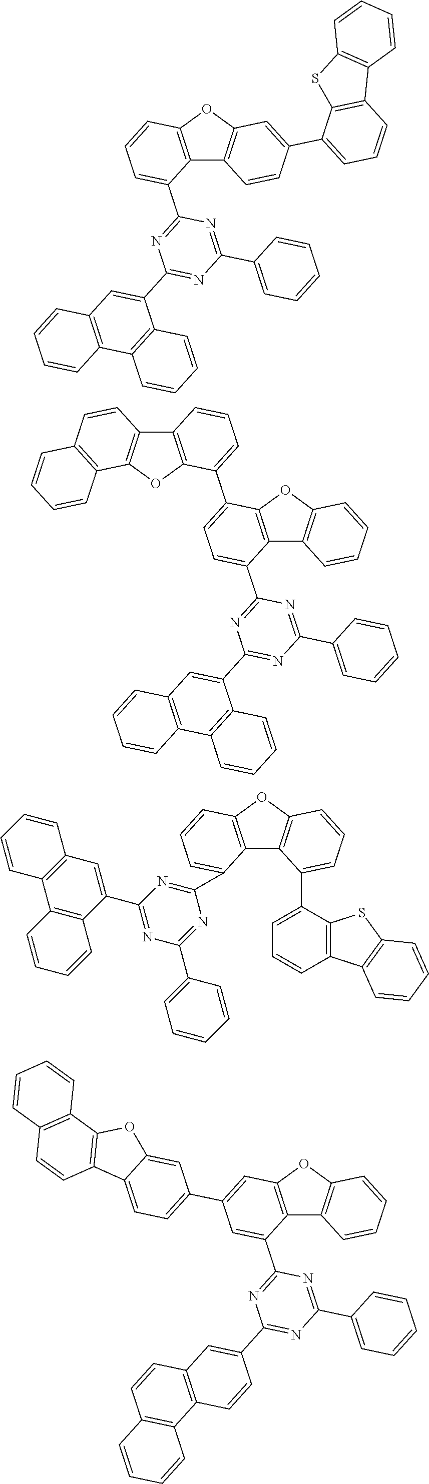

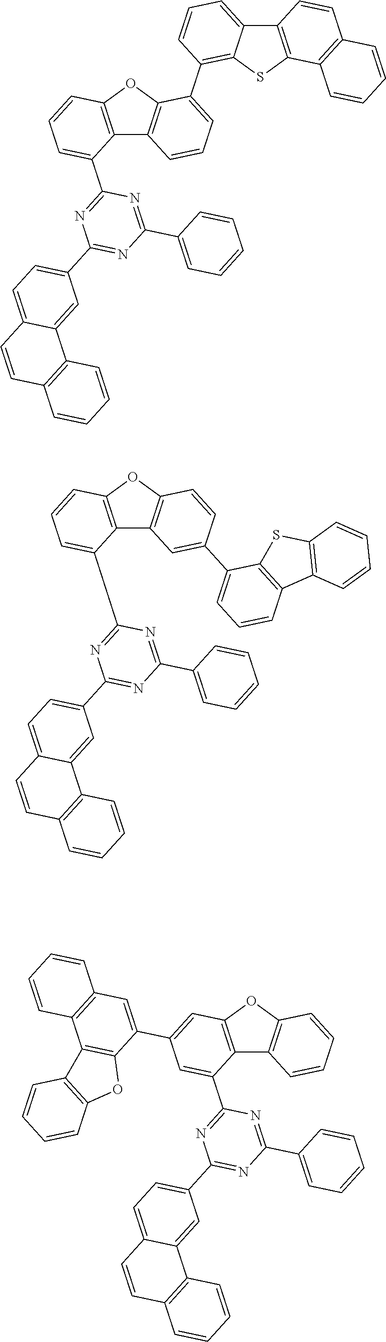 [SMILES: c1ccc(-c2nc(-c3ccc4ccc5ccccc5c4c3)nc(-c3cccc4oc5c(-c6cccc7c6sc6c8ccccc8ccc76)cccc5c34)n2)cc1 ; c1ccc(-c2nc(-c3ccc4ccc5ccccc5c4c3)nc(-c3cccc4oc5ccc(-c6cccc7c6sc6ccccc67)cc5c34)n2)cc1 ; c1ccc(-c2nc(-c3ccc4ccc5ccccc5c4c3)nc(-c3cc(-c4cc5ccccc5c5c4oc4ccccc45)cc4oc5ccccc5c34)n2)cc1]

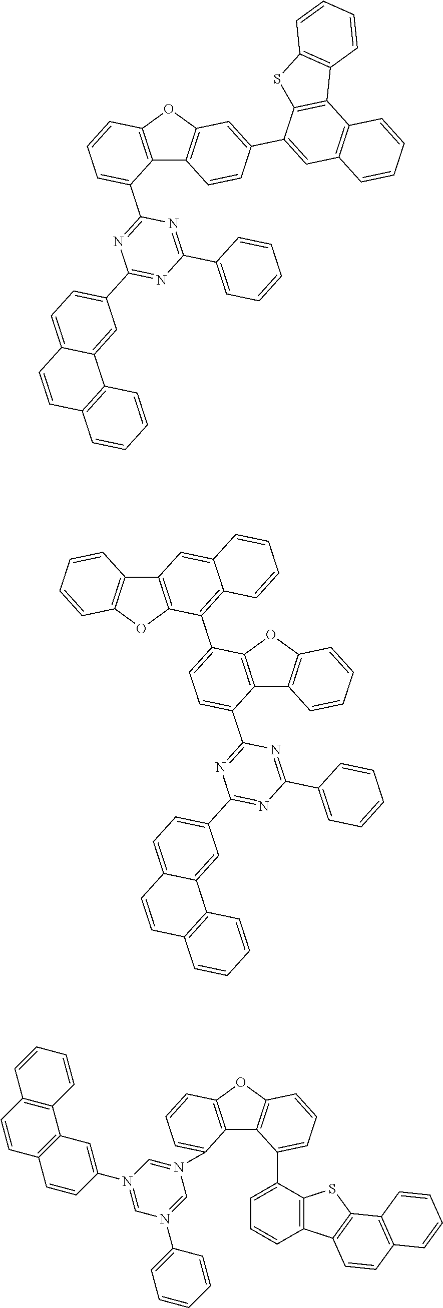

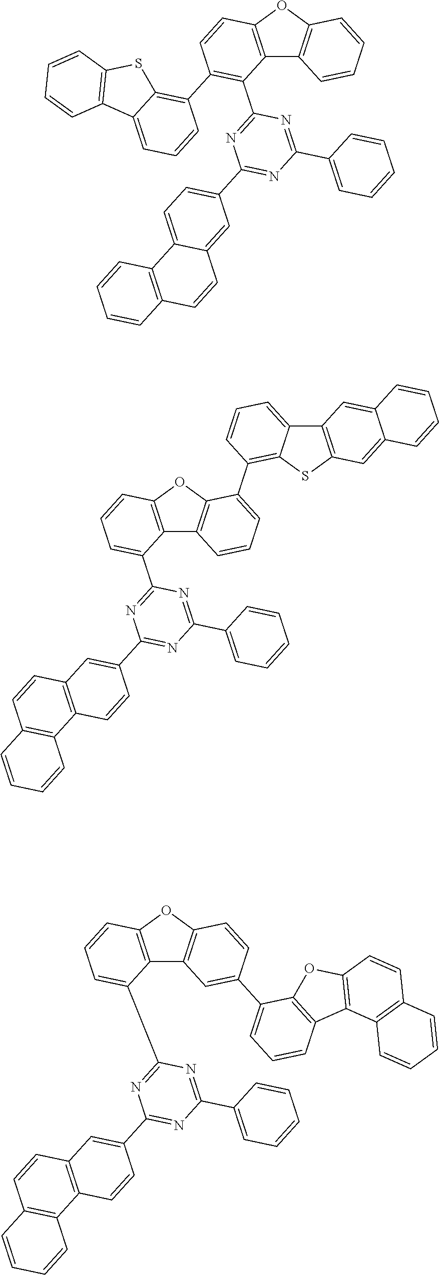 [SMILES: c1ccc(-c2nc(-c3ccc4c(ccc5ccccc54)c3)nc(-c3c(-c4cccc5c4sc4ccccc45)ccc4oc5ccccc5c34)n2)cc1 ; c1ccc(-c2nc(-c3ccc4c(ccc5ccccc54)c3)nc(-c3cccc4oc5c(-c6cccc7c6sc6cc8ccccc8cc67)cccc5c34)n2)cc1 ; c1ccc(-c2nc(-c3ccc4c(ccc5ccccc54)c3)nc(-c3cccc4oc5ccc(-c6cccc7c6oc6ccc8ccccc8c67)cc5c34)n2)cc1]

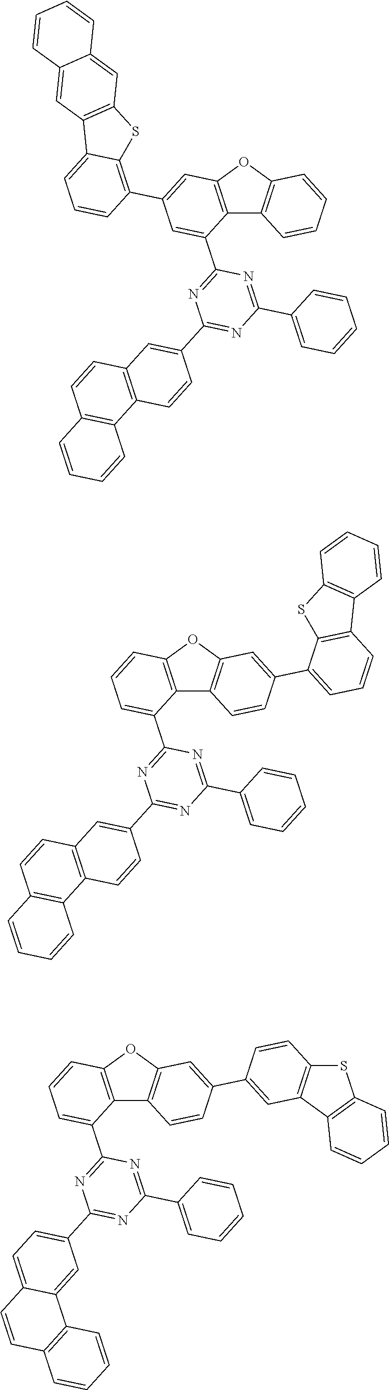 [SMILES: c1ccc(-c2nc(-c3ccc4c(ccc5ccccc54)c3)nc(-c3cc(-c4cccc5c4sc4cc6ccccc6cc45)cc4oc5ccccc5c34)n2)cc1 ; c1ccc(-c2nc(-c3ccc4c(ccc5ccccc54)c3)nc(-c3cccc4oc5cc(-c6cccc7c6sc6ccccc67)ccc5c34)n2)cc1 ; c1ccc(-c2nc(-c3ccc4ccc5ccccc5c4c3)nc(-c3cccc4oc5cc(-c6ccc7sc8ccccc8c7c6)ccc5c34)n2)cc1]

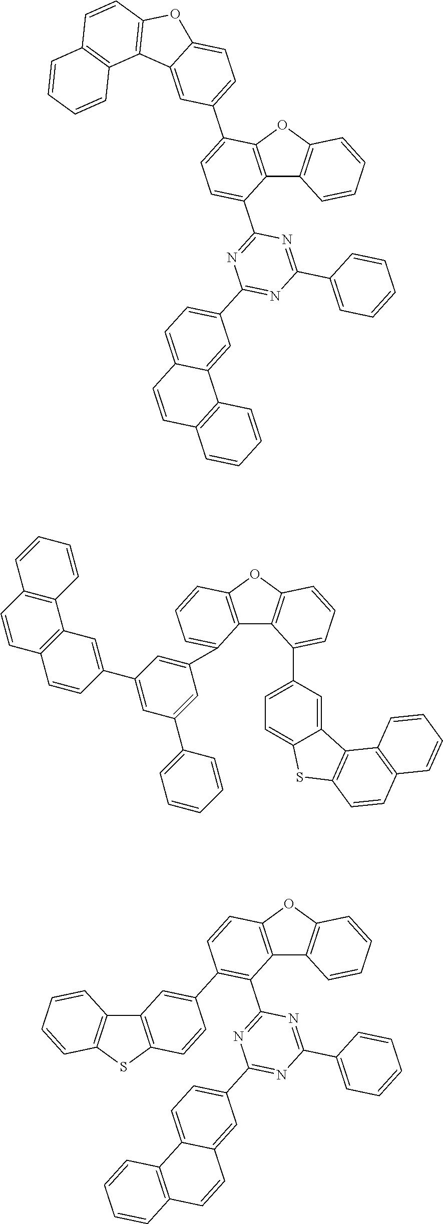 [SMILES: c1ccc(-c2nc(-c3ccc4ccc5ccccc5c4c3)nc(-c3ccc(-c4ccc5oc6ccc7ccccc7c6c5c4)c4oc5ccccc5c34)n2)cc1 ; c1ccc(-c2cc(-c3ccc4ccc5ccccc5c4c3)cc(-c3cccc4oc5cccc(-c6ccc7sc8ccc9ccccc9c8c7c6)c5c34)c2)cc1 ; c1ccc(-c2nc(-c3ccc4c(ccc5ccccc54)c3)nc(-c3c(-c4ccc5sc6ccccc6c5c4)ccc4oc5ccccc5c34)n2)cc1]

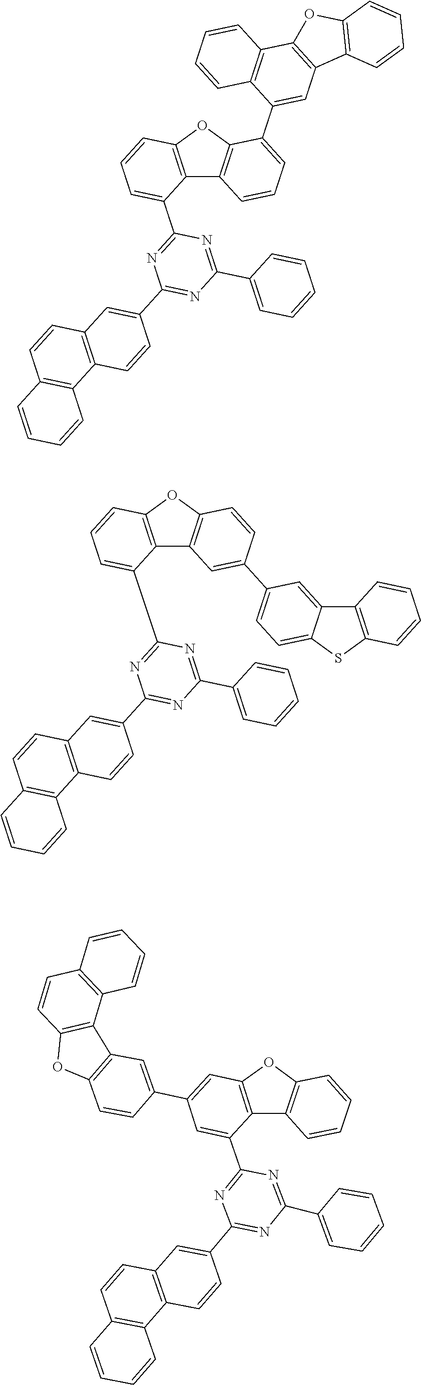 [SMILES: c1ccc(-c2nc(-c3ccc4c(ccc5ccccc54)c3)nc(-c3cccc4oc5c(-c6cc7c8ccccc8oc7c7ccccc67)cccc5c34)n2)cc1 ; c1ccc(-c2nc(-c3ccc4c(ccc5ccccc54)c3)nc(-c3cccc4oc5ccc(-c6ccc7sc8ccccc8c7c6)cc5c34)n2)cc1 ; c1ccc(-c2nc(-c3ccc4c(ccc5ccccc54)c3)nc(-c3cc(-c4ccc5oc6ccc7ccccc7c6c5c4)cc4oc5ccccc5c34)n2)cc1]

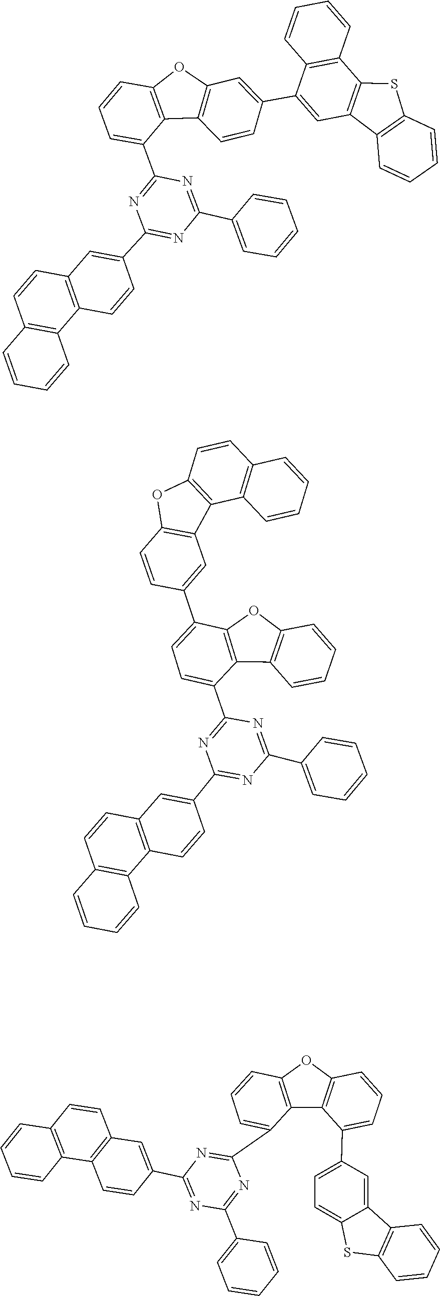 [SMILES: c1ccc(-c2nc(-c3ccc4c(ccc5ccccc54)c3)nc(-c3cccc4oc5cc(-c6cc7c8ccccc8sc7c7ccccc67)ccc5c34)n2)cc1 ; c1ccc(-c2nc(-c3ccc4c(ccc5ccccc54)c3)nc(-c3ccc(-c4ccc5oc6ccc7ccccc7c6c5c4)c4oc5ccccc5c34)n2)cc1 ; c1ccc(-c2nc(-c3ccc4c(ccc5ccccc54)c3)nc(-c3cccc4oc5cccc(-c6ccc7sc8ccccc8c7c6)c5c34)n2)cc1]

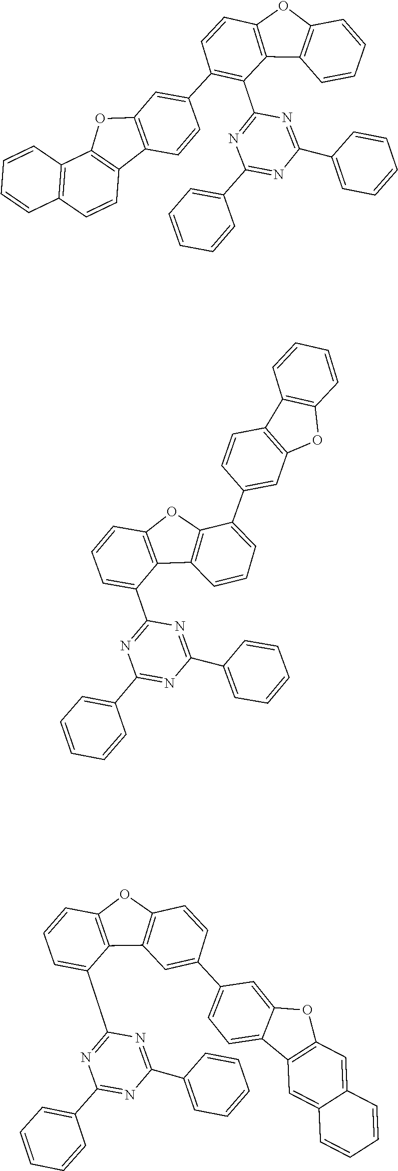 [SMILES: c1ccc(-c2nc(-c3ccccc3)nc(-c3c(-c4ccc5c(c4)oc4c6ccccc6ccc54)ccc4oc5ccccc5c34)n2)cc1 ; c1ccc(-c2nc(-c3ccccc3)nc(-c3cccc4oc5c(-c6ccc7c(c6)oc6ccccc67)cccc5c34)n2)cc1 ; c1ccc(-c2nc(-c3ccccc3)nc(-c3cccc4oc5ccc(-c6ccc7c(c6)oc6cc8ccccc8cc67)cc5c34)n2)cc1]

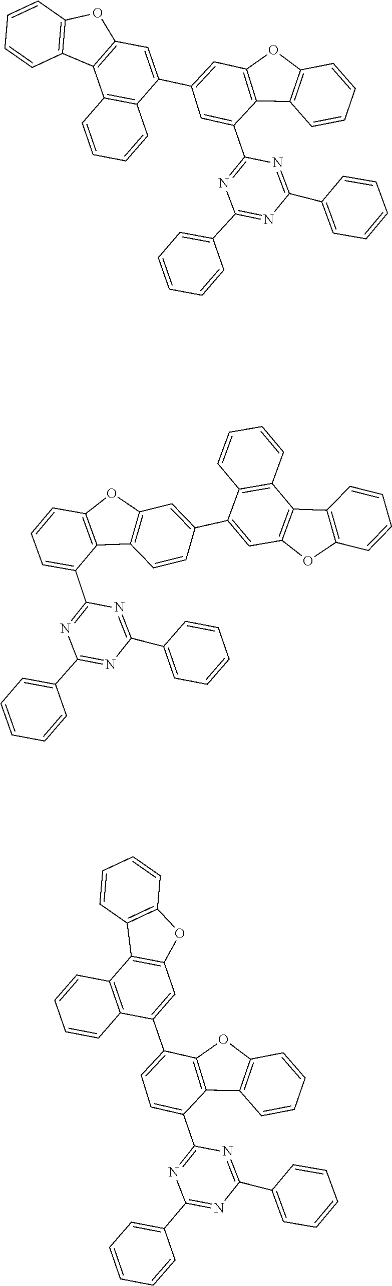 [SMILES: c1ccc(-c2nc(-c3ccccc3)nc(-c3cc(-c4cc5oc6ccccc6c5c5ccccc45)cc4oc5ccccc5c34)n2)cc1 ; c1ccc(-c2nc(-c3ccccc3)nc(-c3cccc4oc5cc(-c6cc7oc8ccccc8c7c7ccccc67)ccc5c34)n2)cc1 ; c1ccc(-c2nc(-c3ccccc3)nc(-c3ccc(-c4cc5oc6ccccc6c5c5ccccc45)c4oc5ccccc5c34)n2)cc1]

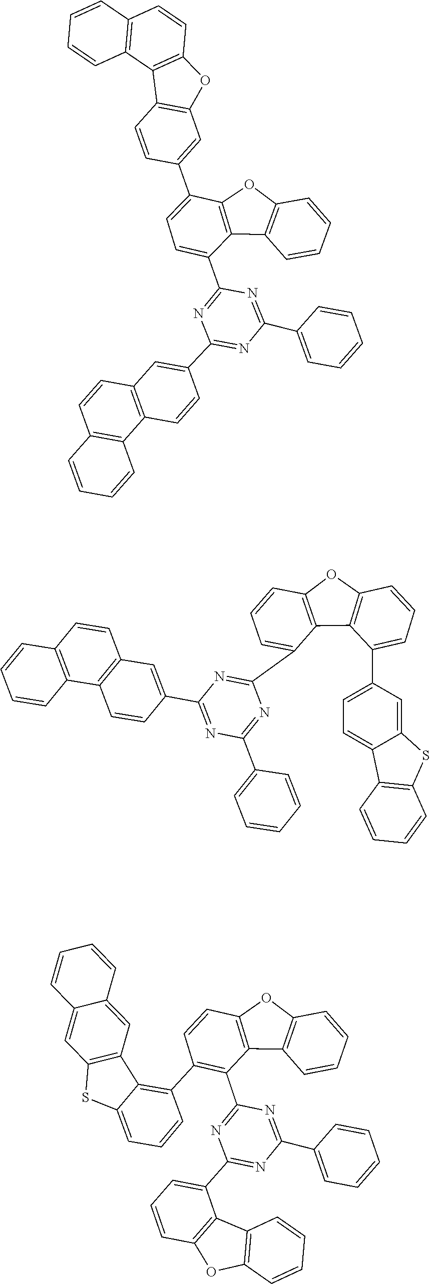 [SMILES: c1ccc(-c2nc(-c3ccc4c(ccc5ccccc54)c3)nc(-c3ccc(-c4ccc5c(c4)oc4ccc6ccccc6c45)c4oc5ccccc5c34)n2)cc1 ; c1ccc(-c2nc(-c3ccc4c(ccc5ccccc54)c3)nc(-c3cccc4oc5cccc(-c6ccc7c(c6)sc6ccccc67)c5c34)n2)cc1 ; c1ccc(-c2nc(-c3cccc4oc5ccccc5c34)nc(-c3c(-c4cccc5sc6cc7ccccc7cc6c45)ccc4oc5ccccc5c34)n2)cc1]

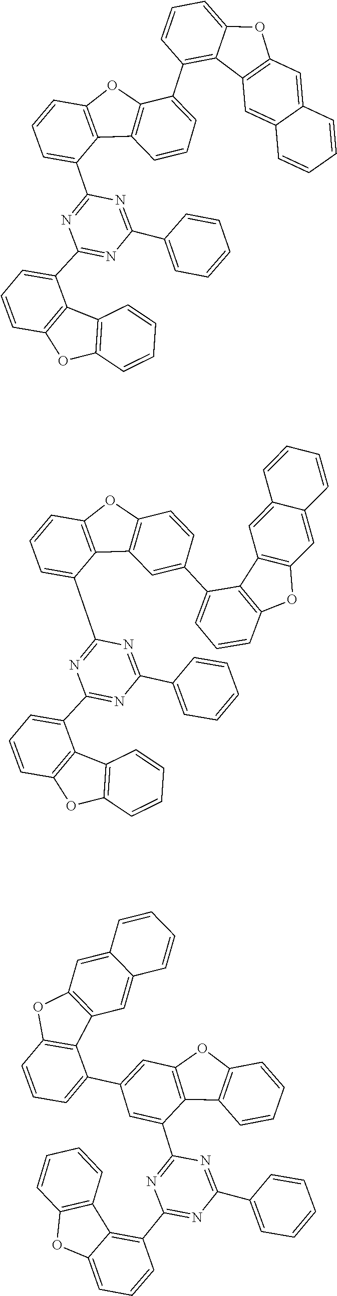 [SMILES: c1ccc(-c2nc(-c3cccc4oc5ccccc5c34)nc(-c3cccc4oc5c(-c6cccc7oc8cc9ccccc9cc8c67)cccc5c34)n2)cc1 ; c1ccc(-c2nc(-c3cccc4oc5ccccc5c34)nc(-c3cccc4oc5ccc(-c6cccc7oc8cc9ccccc9cc8c67)cc5c34)n2)cc1 ; c1ccc(-c2nc(-c3cccc4oc5ccccc5c34)nc(-c3cc(-c4cccc5oc6cc7ccccc7cc6c45)cc4oc5ccccc5c34)n2)cc1]

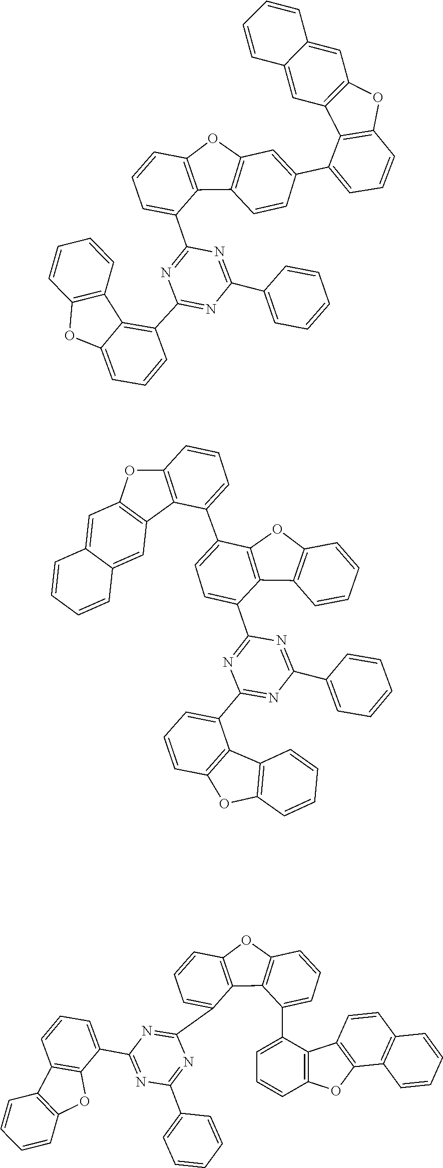 [SMILES: c1ccc(-c2nc(-c3cccc4oc5ccccc5c34)nc(-c3cccc4oc5cc(-c6cccc7oc8cc9ccccc9cc8c67)ccc5c34)n2)cc1 ; c1ccc(-c2nc(-c3cccc4oc5ccccc5c34)nc(-c3ccc(-c4cccc5oc6cc7ccccc7cc6c45)c4oc5ccccc5c34)n2)cc1 ; c1ccc(-c2nc(-c3cccc4c3oc3ccccc34)nc(-c3cccc4oc5cccc(-c6cccc7oc8c9ccccc9ccc8c67)c5c34)n2)cc1]

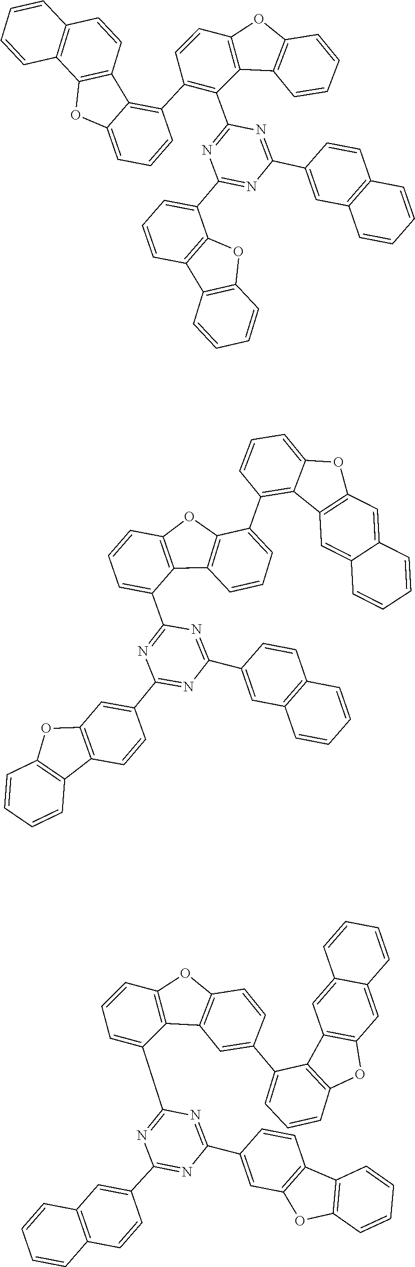 [SMILES: c1ccc2cc(-c3nc(-c4cccc5c4oc4ccccc45)nc(-c4c(-c5cccc6oc7c8ccccc8ccc7c56)ccc5oc6ccccc6c45)n3)ccc2c1 ; c1ccc2cc(-c3nc(-c4ccc5c(c4)oc4ccccc45)nc(-c4cccc5oc6c(-c7cccc8oc9cc%10ccccc%10cc9c78)cccc6c45)n3)ccc2c1 ; c1ccc2cc(-c3nc(-c4ccc5c(c4)oc4ccccc45)nc(-c4cccc5oc6ccc(-c7cccc8oc9cc%10ccccc%10cc9c78)cc6c45)n3)ccc2c1]

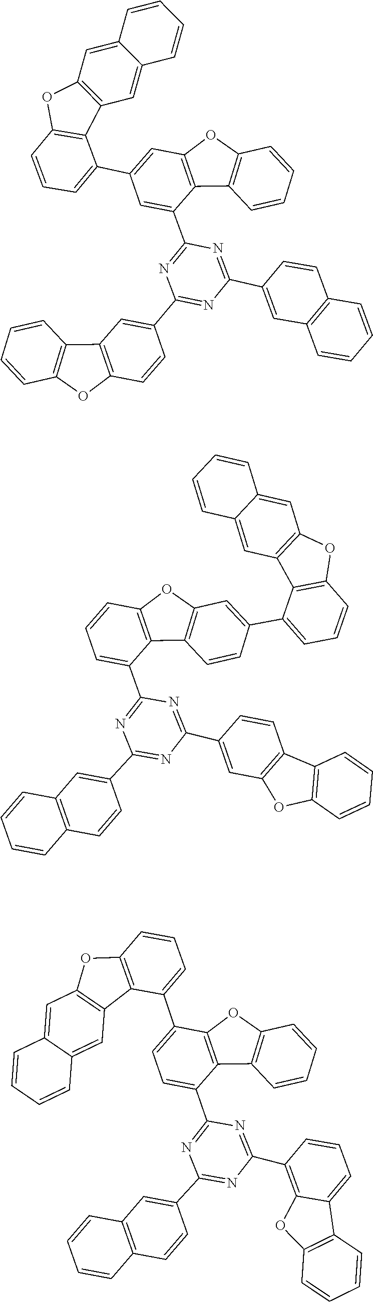 [SMILES: c1ccc2cc(-c3nc(-c4ccc5oc6ccccc6c5c4)nc(-c4cc(-c5cccc6oc7cc8ccccc8cc7c56)cc5oc6ccccc6c45)n3)ccc2c1 ; c1ccc2cc(-c3nc(-c4ccc5c(c4)oc4ccccc45)nc(-c4cccc5oc6cc(-c7cccc8oc9cc%10ccccc%10cc9c78)ccc6c45)n3)ccc2c1 ; c1ccc2cc(-c3nc(-c4cccc5c4oc4ccccc45)nc(-c4ccc(-c5cccc6oc7cc8ccccc8cc7c56)c5oc6ccccc6c45)n3)ccc2c1]

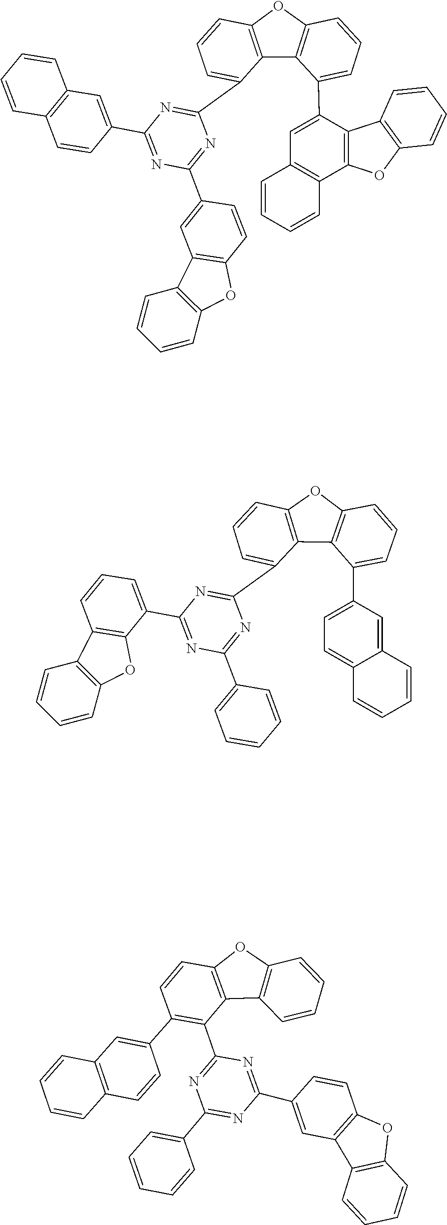 [SMILES: c1ccc2cc(-c3nc(-c4ccc5oc6ccccc6c5c4)nc(-c4cccc5oc6cccc(-c7cc8ccccc8c8oc9ccccc9c78)c6c45)n3)ccc2c1 ; c1ccc(-c2nc(-c3cccc4c3oc3ccccc34)nc(-c3cccc4oc5cccc(-c6ccc7ccccc7c6)c5c34)n2)cc1 ; c1ccc(-c2nc(-c3ccc4oc5ccccc5c4c3)nc(-c3c(-c4ccc5ccccc5c4)ccc4oc5ccccc5c34)n2)cc1]

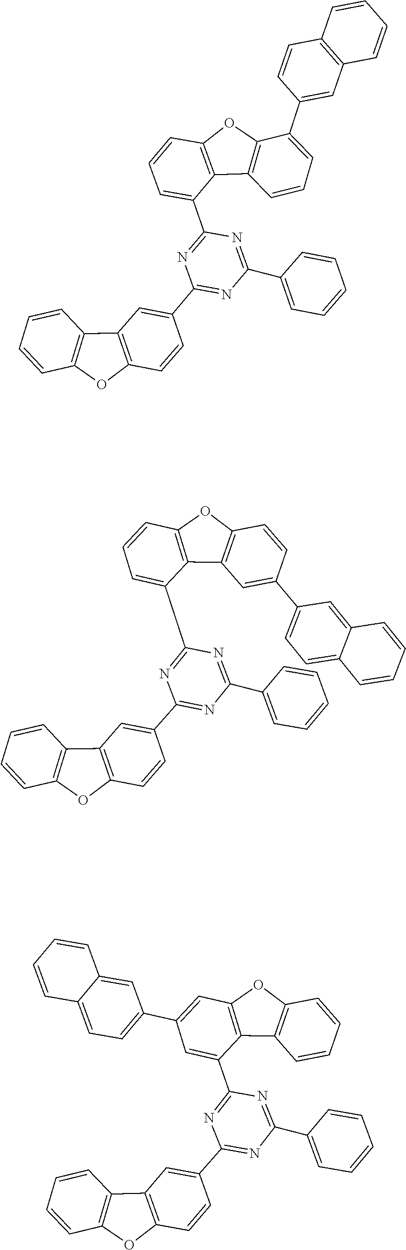 [SMILES: c1ccc(-c2nc(-c3ccc4oc5ccccc5c4c3)nc(-c3cccc4oc5c(-c6ccc7ccccc7c6)cccc5c34)n2)cc1 ; c1ccc(-c2nc(-c3ccc4oc5ccccc5c4c3)nc(-c3cccc4oc5ccc(-c6ccc7ccccc7c6)cc5c34)n2)cc1 ; c1ccc(-c2nc(-c3ccc4oc5ccccc5c4c3)nc(-c3cc(-c4ccc5ccccc5c4)cc4oc5ccccc5c34)n2)cc1]

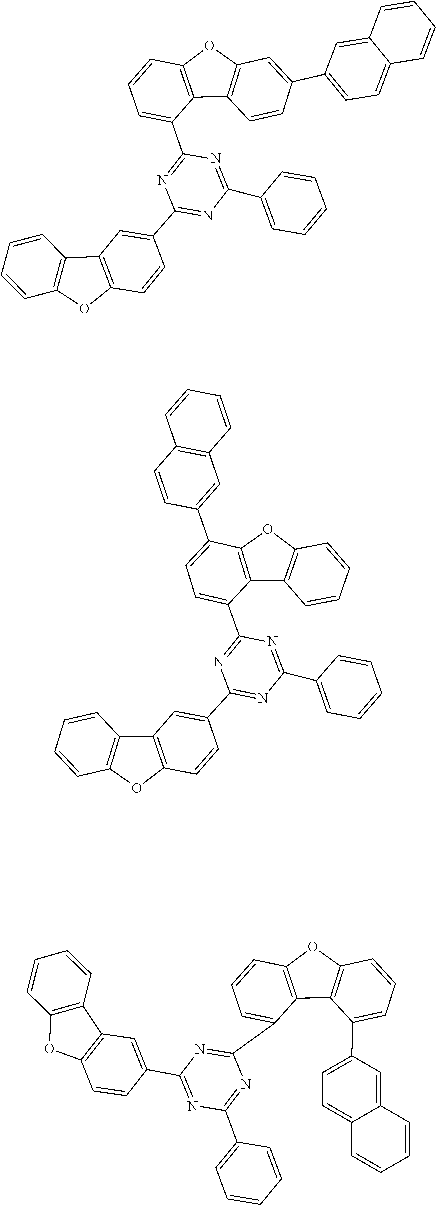 [SMILES: c1ccc(-c2nc(-c3ccc4oc5ccccc5c4c3)nc(-c3cccc4oc5cc(-c6ccc7ccccc7c6)ccc5c34)n2)cc1 ; c1ccc(-c2nc(-c3ccc4oc5ccccc5c4c3)nc(-c3ccc(-c4ccc5ccccc5c4)c4oc5ccccc5c34)n2)cc1 ; c1ccc(-c2nc(-c3ccc4oc5ccccc5c4c3)nc(-c3cccc4oc5cccc(-c6ccc7ccccc7c6)c5c34)n2)cc1]

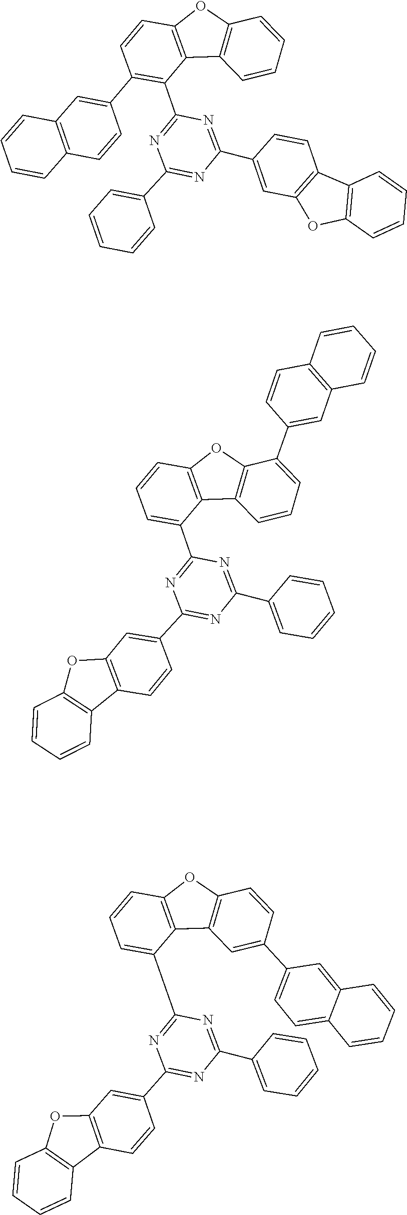 [SMILES: c1ccc(-c2nc(-c3ccc4c(c3)oc3ccccc34)nc(-c3c(-c4ccc5ccccc5c4)ccc4oc5ccccc5c34)n2)cc1 ; c1ccc(-c2nc(-c3ccc4c(c3)oc3ccccc34)nc(-c3cccc4oc5c(-c6ccc7ccccc7c6)cccc5c34)n2)cc1 ; c1ccc(-c2nc(-c3ccc4c(c3)oc3ccccc34)nc(-c3cccc4oc5ccc(-c6ccc7ccccc7c6)cc5c34)n2)cc1]

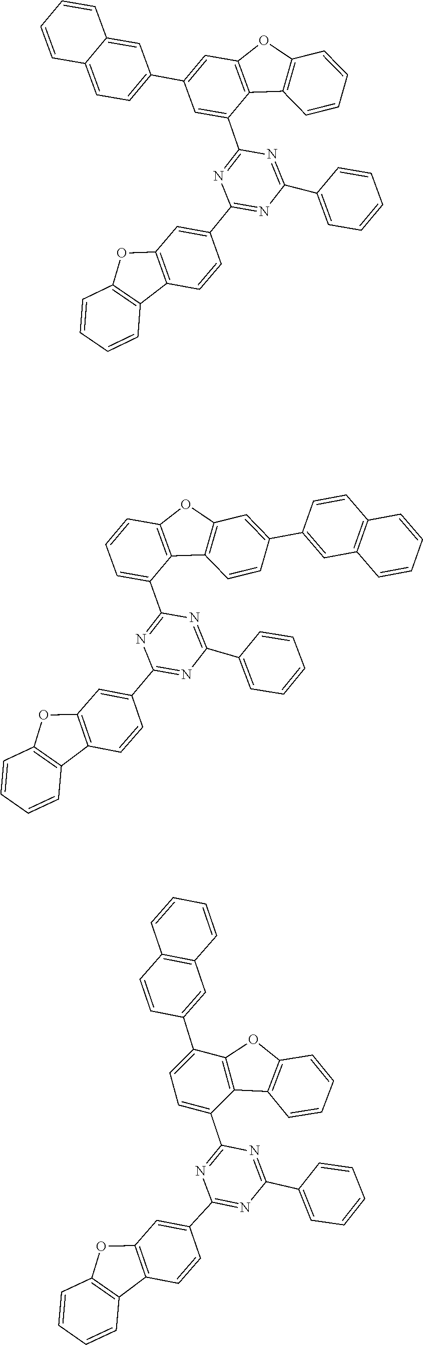 [SMILES: c1ccc(-c2nc(-c3ccc4c(c3)oc3ccccc34)nc(-c3cc(-c4ccc5ccccc5c4)cc4oc5ccccc5c34)n2)cc1 ; c1ccc(-c2nc(-c3ccc4c(c3)oc3ccccc34)nc(-c3cccc4oc5cc(-c6ccc7ccccc7c6)ccc5c34)n2)cc1 ; c1ccc(-c2nc(-c3ccc4c(c3)oc3ccccc34)nc(-c3ccc(-c4ccc5ccccc5c4)c4oc5ccccc5c34)n2)cc1]

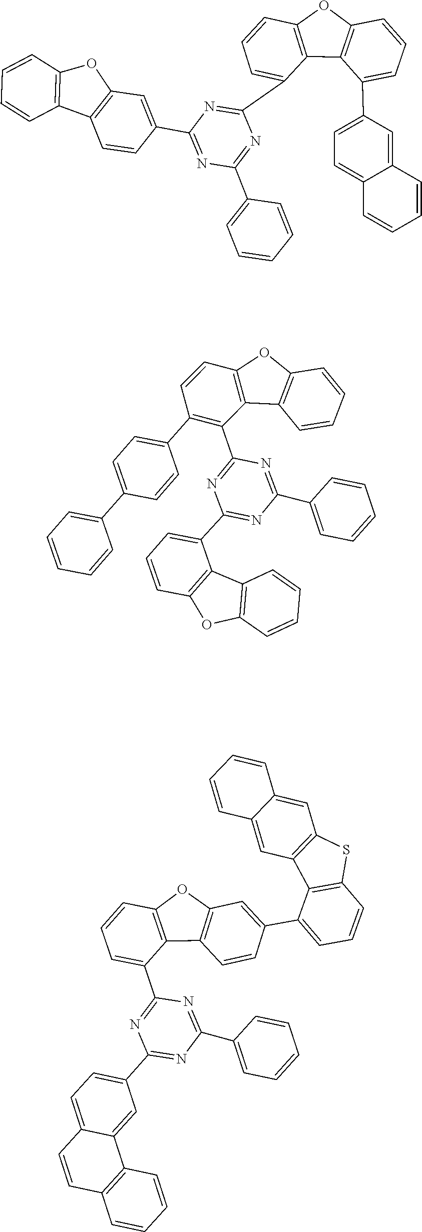 [SMILES: c1ccc(-c2nc(-c3ccc4c(c3)oc3ccccc34)nc(-c3cccc4oc5cccc(-c6ccc7ccccc7c6)c5c34)n2)cc1 ; c1ccc(-c2ccc(-c3ccc4oc5ccccc5c4c3-c3nc(-c4ccccc4)nc(-c4cccc5oc6ccccc6c45)n3)cc2)cc1 ; c1ccc(-c2nc(-c3ccc4ccc5ccccc5c4c3)nc(-c3cccc4oc5cc(-c6cccc7sc8cc9ccccc9cc8c67)ccc5c34)n2)cc1]

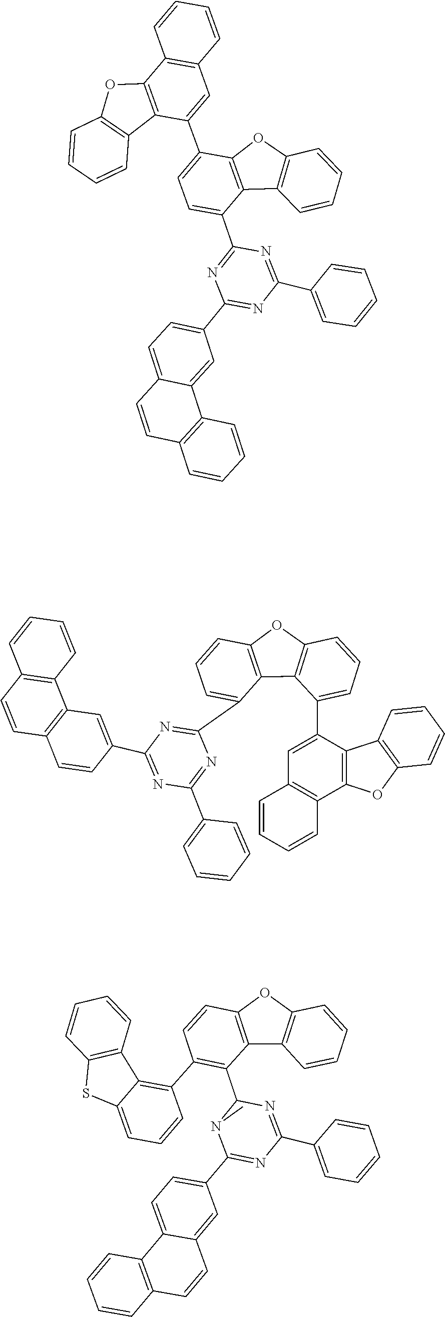

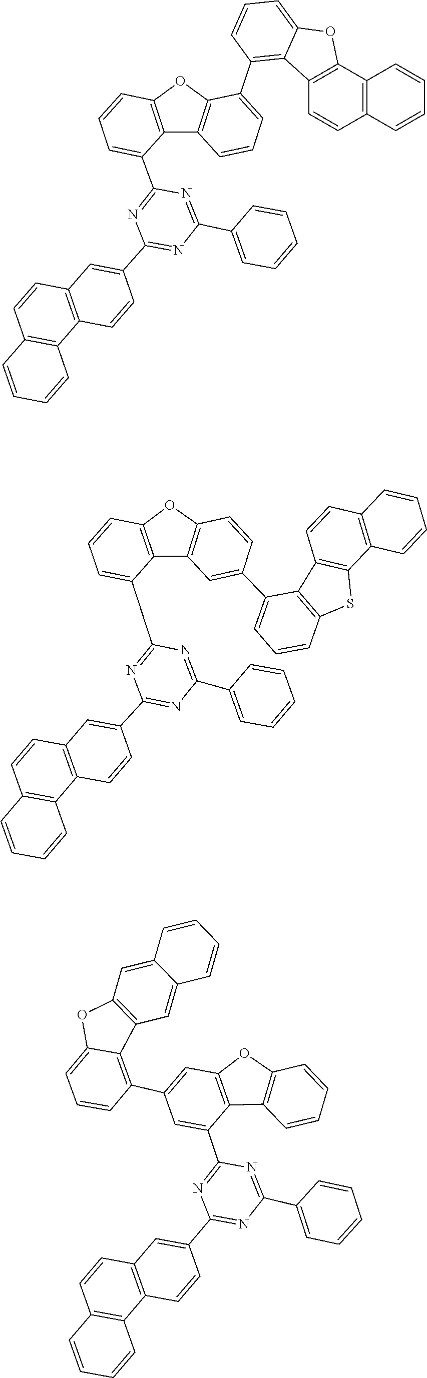 [SMILES: c1ccc(-c2nc(-c3ccc4c(ccc5ccccc54)c3)nc(-c3cccc4oc5c(-c6cccc7oc8c9ccccc9ccc8c67)cccc5c34)n2)cc1 ; c1ccc(-c2nc(-c3ccc4c(ccc5ccccc54)c3)nc(-c3cccc4oc5ccc(-c6cccc7sc8c9ccccc9ccc8c67)cc5c34)n2)cc1 ; c1ccc(-c2nc(-c3ccc4c(ccc5ccccc54)c3)nc(-c3cc(-c4cccc5oc6cc7ccccc7cc6c45)cc4oc5ccccc5c34)n2)cc1]

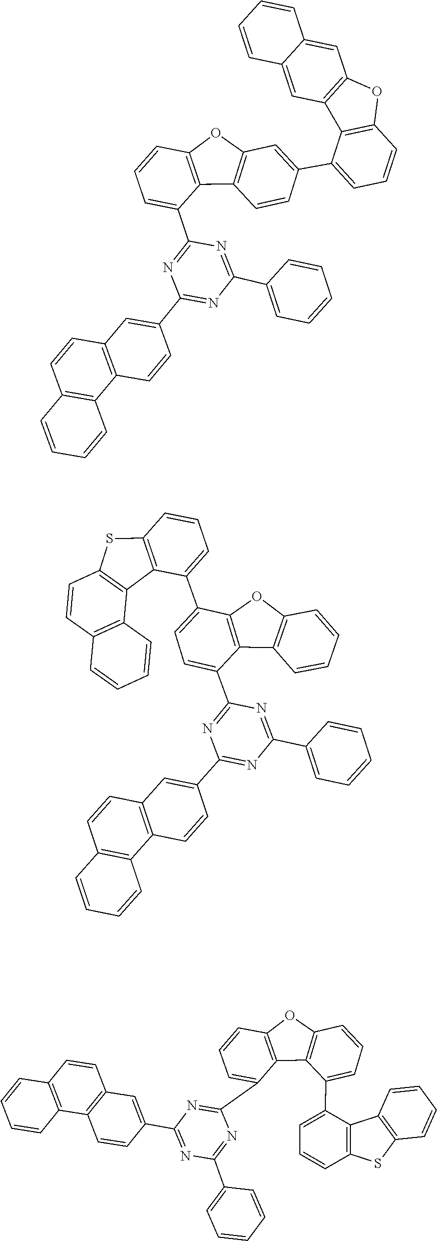 [SMILES: c1ccc(-c2nc(-c3ccc4c(ccc5ccccc54)c3)nc(-c3cccc4oc5cc(-c6cccc7oc8cc9ccccc9cc8c67)ccc5c34)n2)cc1 ; c1ccc(-c2nc(-c3ccc4c(ccc5ccccc54)c3)nc(-c3ccc(-c4cccc5sc6ccc7ccccc7c6c45)c4oc5ccccc5c34)n2)cc1 ; c1ccc(-c2nc(-c3ccc4c(ccc5ccccc54)c3)nc(-c3cccc4oc5cccc(-c6cccc7sc8ccccc8c67)c5c34)n2)cc1]

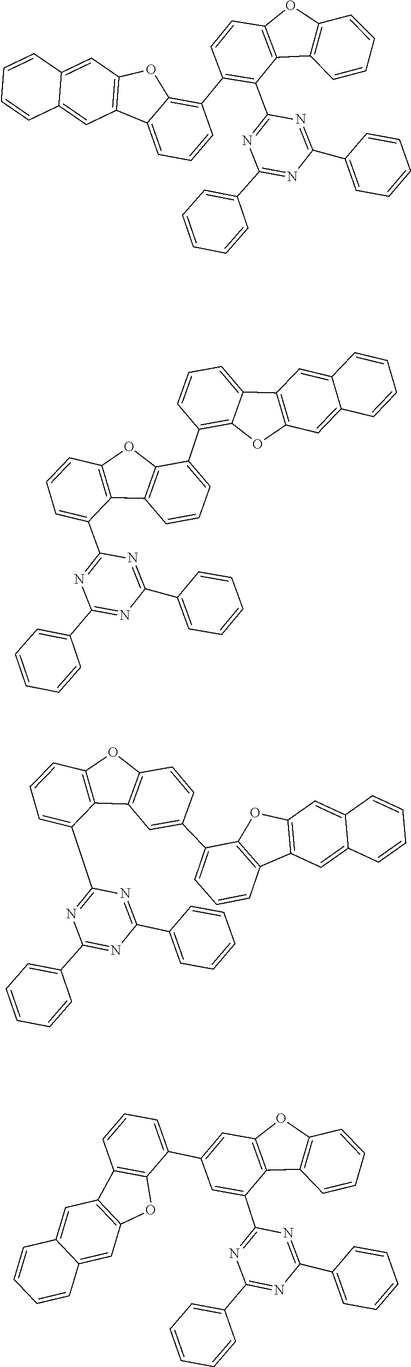 [SMILES: c1ccc(-c2nc(-c3ccccc3)nc(-c3c(-c4cccc5c4oc4cc6ccccc6cc45)ccc4oc5ccccc5c34)n2)cc1 ; c1ccc(-c2nc(-c3ccccc3)nc(-c3cccc4oc5c(-c6cccc7c6oc6cc8ccccc8cc67)cccc5c34)n2)cc1 ; c1ccc(-c2nc(-c3ccccc3)nc(-c3cccc4oc5ccc(-c6cccc7c6oc6cc8ccccc8cc67)cc5c34)n2)cc1 ; c1ccc(-c2nc(-c3ccccc3)nc(-c3cc(-c4cccc5c4oc4cc6ccccc6cc45)cc4oc5ccccc5c34)n2)cc1]

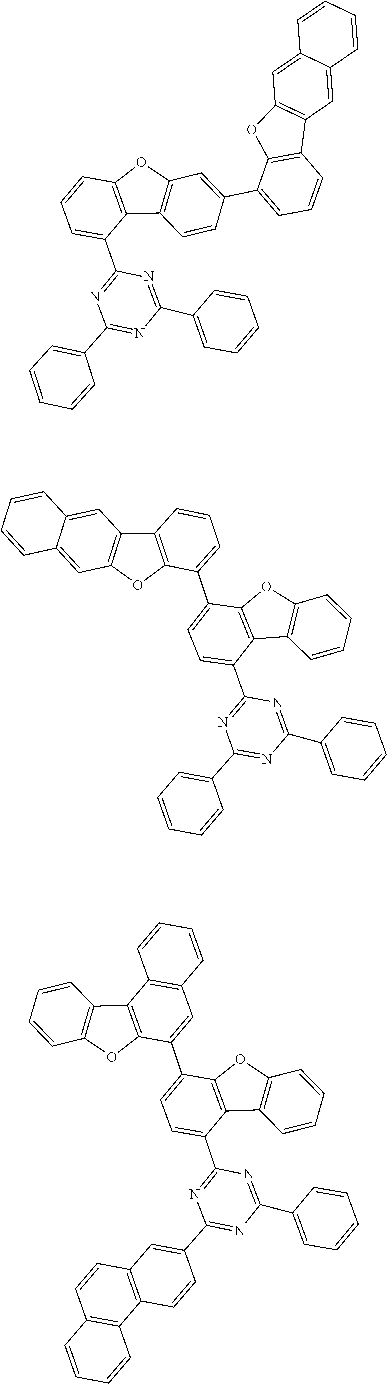 [SMILES: c1ccc(-c2nc(-c3ccccc3)nc(-c3cccc4oc5cc(-c6cccc7c6oc6cc8ccccc8cc67)ccc5c34)n2)cc1 ; c1ccc(-c2nc(-c3ccccc3)nc(-c3ccc(-c4cccc5c4oc4cc6ccccc6cc45)c4oc5ccccc5c34)n2)cc1 ; c1ccc(-c2nc(-c3ccc4c(ccc5ccccc54)c3)nc(-c3ccc(-c4cc5ccccc5c5c4oc4ccccc45)c4oc5ccccc5c34)n2)cc1]

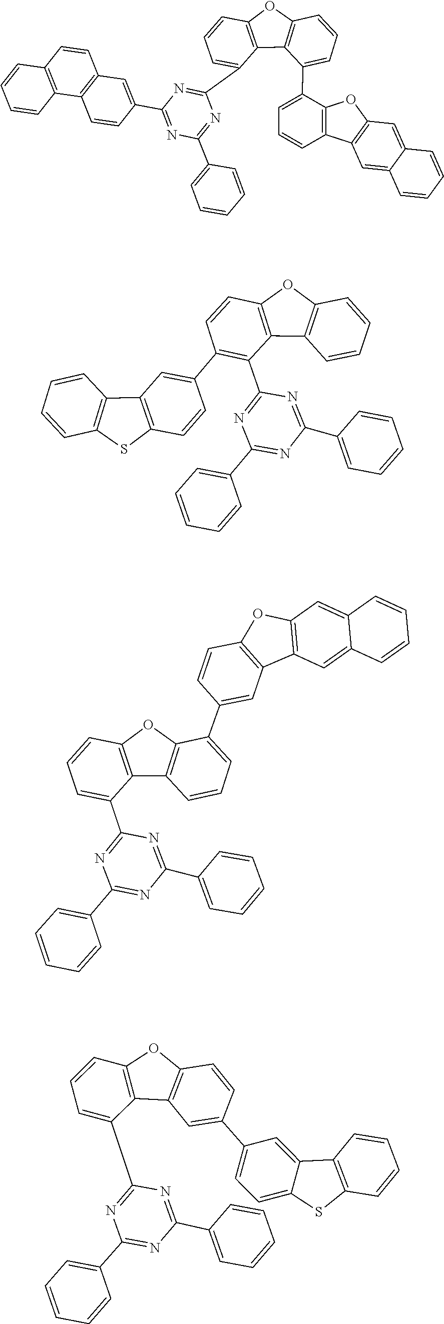 [SMILES: c1ccc(-c2nc(-c3ccc4c(ccc5ccccc54)c3)nc(-c3cccc4oc5cccc(-c6cccc7c6oc6cc8ccccc8cc67)c5c34)n2)cc1 ; c1ccc(-c2nc(-c3ccccc3)nc(-c3c(-c4ccc5sc6ccccc6c5c4)ccc4oc5ccccc5c34)n2)cc1 ; c1ccc(-c2nc(-c3ccccc3)nc(-c3cccc4oc5c(-c6ccc7oc8cc9ccccc9cc8c7c6)cccc5c34)n2)cc1 ; c1ccc(-c2nc(-c3ccccc3)nc(-c3cccc4oc5ccc(-c6ccc7sc8ccccc8c7c6)cc5c34)n2)cc1]

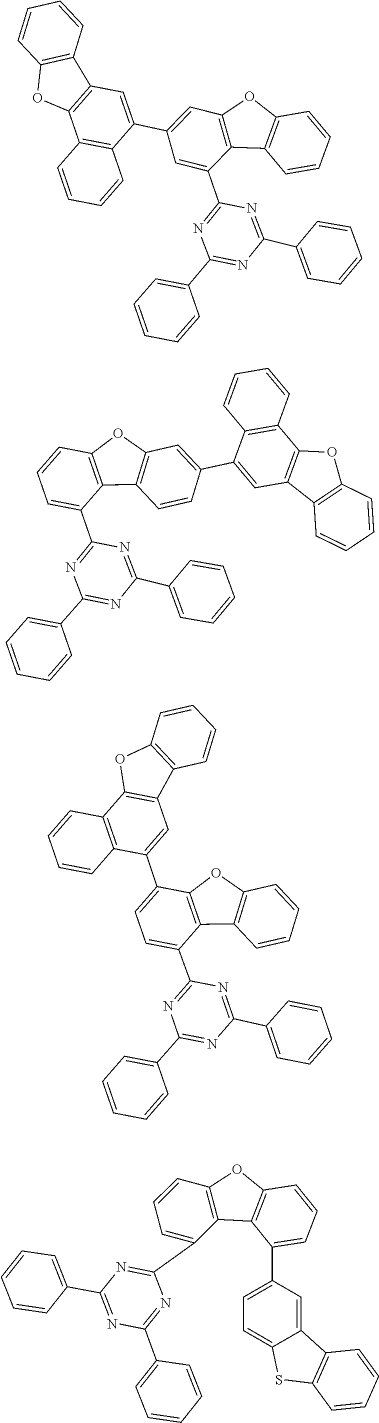 [SMILES: c1ccc(-c2nc(-c3ccccc3)nc(-c3cc(-c4cc5c6ccccc6oc5c5ccccc45)cc4oc5ccccc5c34)n2)cc1 ; c1ccc(-c2nc(-c3ccccc3)nc(-c3cccc4oc5cc(-c6cc7c8ccccc8oc7c7ccccc67)ccc5c34)n2)cc1 ; c1ccc(-c2nc(-c3ccccc3)nc(-c3ccc(-c4cc5c6ccccc6oc5c5ccccc45)c4oc5ccccc5c34)n2)cc1 ; c1ccc(-c2nc(-c3ccccc3)nc(-c3cccc4oc5cccc(-c6ccc7sc8ccccc8c7c6)c5c34)n2)cc1]

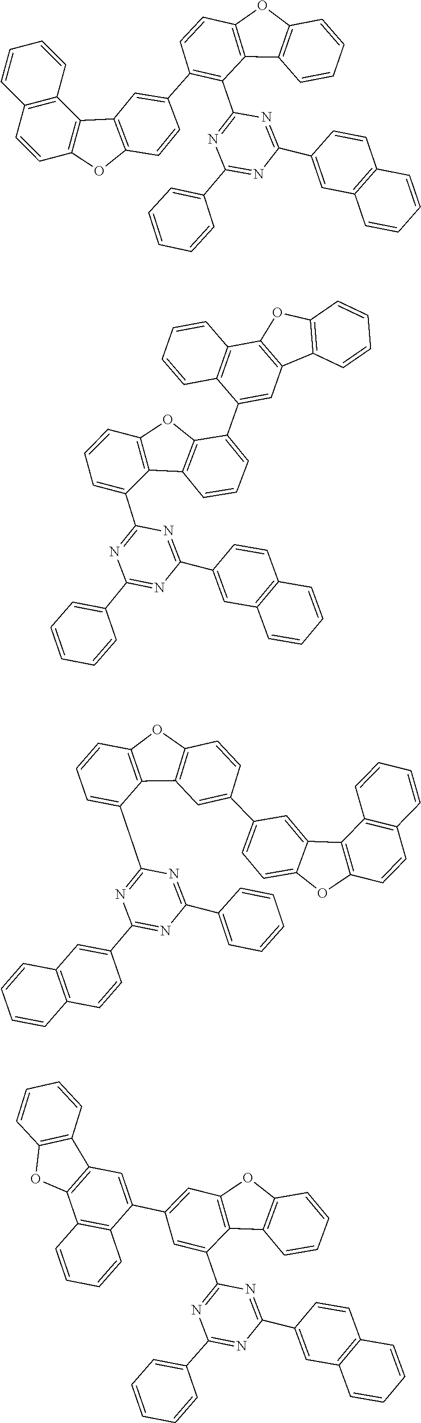 [SMILES: c1ccc(-c2nc(-c3ccc4ccccc4c3)nc(-c3c(-c4ccc5oc6ccc7ccccc7c6c5c4)ccc4oc5ccccc5c34)n2)cc1 ; c1ccc(-c2nc(-c3ccc4ccccc4c3)nc(-c3cccc4oc5c(-c6cc7c8ccccc8oc7c7ccccc67)cccc5c34)n2)cc1 ; c1ccc(-c2nc(-c3ccc4ccccc4c3)nc(-c3cccc4oc5ccc(-c6ccc7oc8ccc9ccccc9c8c7c6)cc5c34)n2)cc1 ; c1ccc(-c2nc(-c3ccc4ccccc4c3)nc(-c3cc(-c4cc5c6ccccc6oc5c5ccccc45)cc4oc5ccccc5c34)n2)cc1]

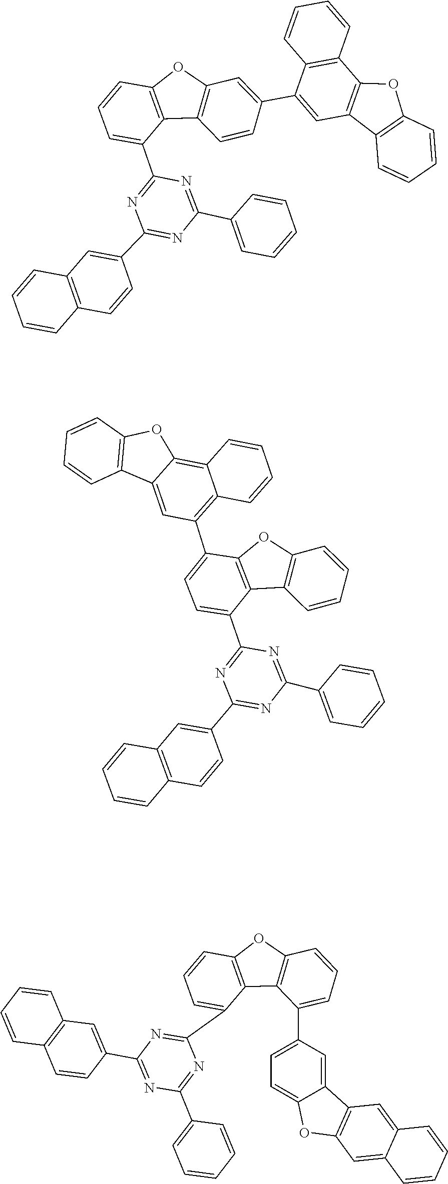 [SMILES: c1ccc(-c2nc(-c3ccc4ccccc4c3)nc(-c3cccc4oc5cc(-c6cc7c8ccccc8oc7c7ccccc67)ccc5c34)n2)cc1 ; c1ccc(-c2nc(-c3ccc4ccccc4c3)nc(-c3ccc(-c4cc5c6ccccc6oc5c5ccccc45)c4oc5ccccc5c34)n2)cc1 ; c1ccc(-c2nc(-c3ccc4ccccc4c3)nc(-c3cccc4oc5cccc(-c6ccc7oc8cc9ccccc9cc8c7c6)c5c34)n2)cc1]

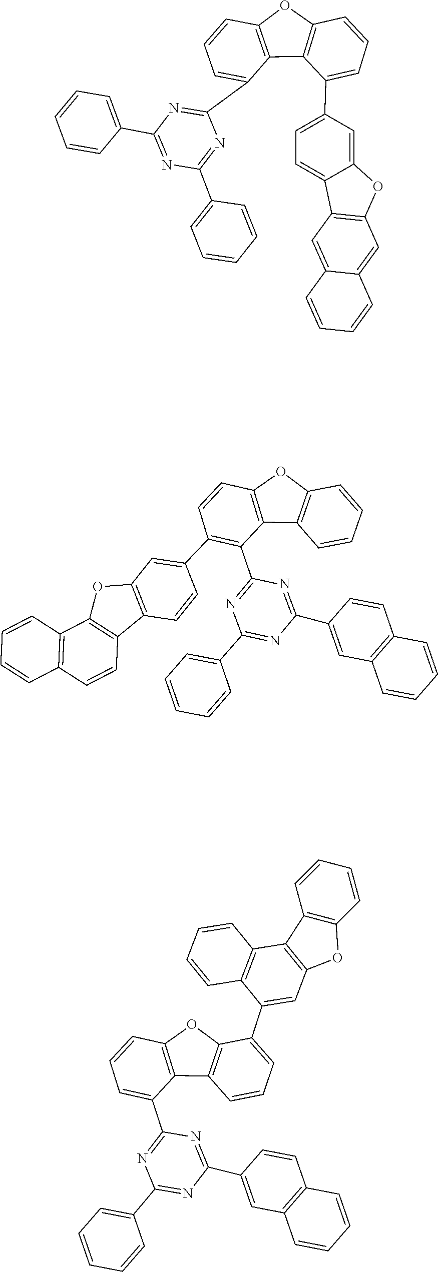 [SMILES: c1ccc(-c2nc(-c3ccccc3)nc(-c3cccc4oc5cccc(-c6ccc7c(c6)oc6cc8ccccc8cc67)c5c34)n2)cc1 ; c1ccc(-c2nc(-c3ccc4ccccc4c3)nc(-c3c(-c4ccc5c(c4)oc4c6ccccc6ccc54)ccc4oc5ccccc5c34)n2)cc1 ; c1ccc(-c2nc(-c3ccc4ccccc4c3)nc(-c3cccc4oc5c(-c6cc7oc8ccccc8c7c7ccccc67)cccc5c34)n2)cc1]

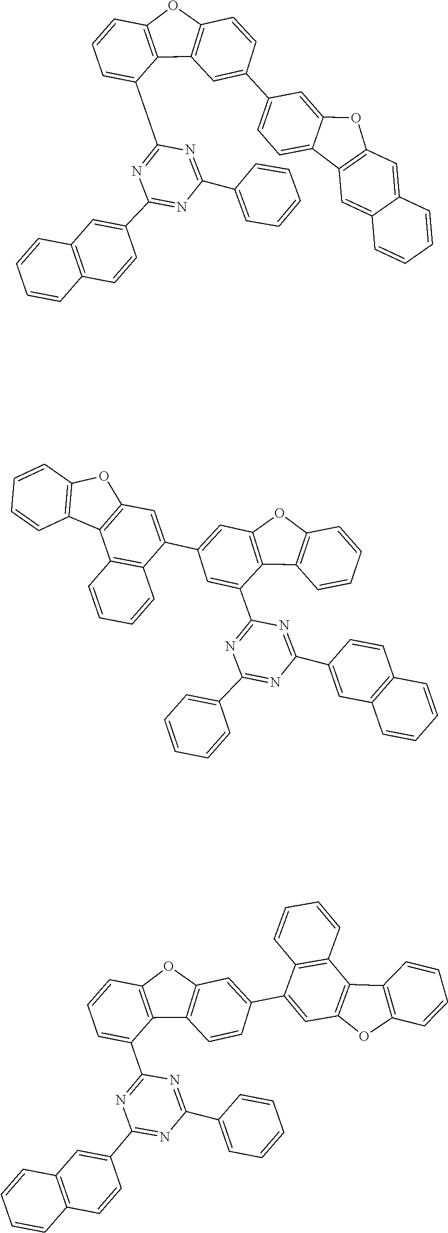 [SMILES: c1ccc(-c2nc(-c3ccc4ccccc4c3)nc(-c3cccc4oc5ccc(-c6ccc7c(c6)oc6cc8ccccc8cc67)cc5c34)n2)cc1 ; c1ccc(-c2nc(-c3ccc4ccccc4c3)nc(-c3cc(-c4cc5oc6ccccc6c5c5ccccc45)cc4oc5ccccc5c34)n2)cc1 ; c1ccc(-c2nc(-c3ccc4ccccc4c3)nc(-c3cccc4oc5cc(-c6cc7oc8ccccc8c7c7ccccc67)ccc5c34)n2)cc1]

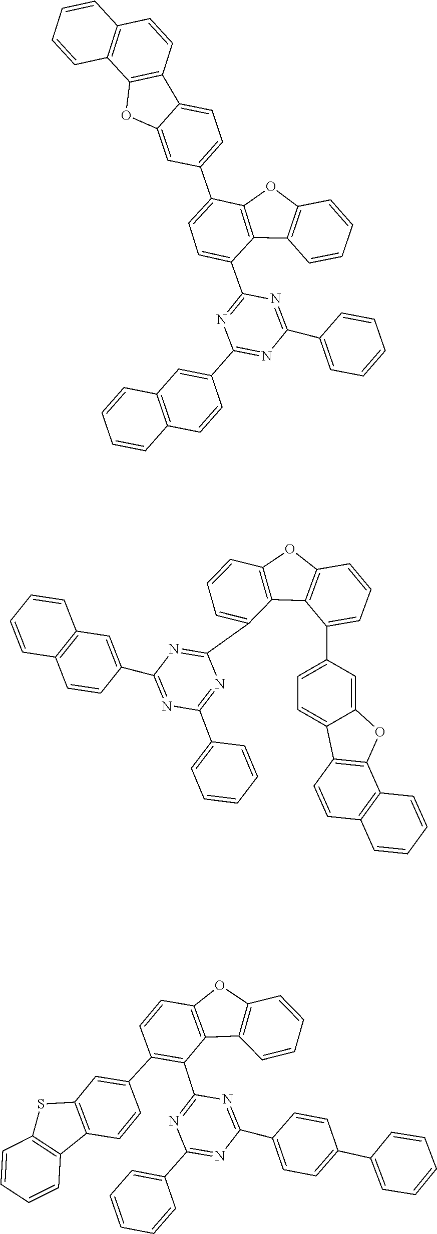 [SMILES: c1ccc(-c2nc(-c3ccc4ccccc4c3)nc(-c3ccc(-c4ccc5c(c4)oc4c6ccccc6ccc54)c4oc5ccccc5c34)n2)cc1 ; c1ccc(-c2nc(-c3ccc4ccccc4c3)nc(-c3cccc4oc5cccc(-c6ccc7c(c6)oc6c8ccccc8ccc76)c5c34)n2)cc1 ; c1ccc(-c2ccc(-c3nc(-c4ccccc4)nc(-c4c(-c5ccc6c(c5)sc5ccccc56)ccc5oc6ccccc6c45)n3)cc2)cc1]

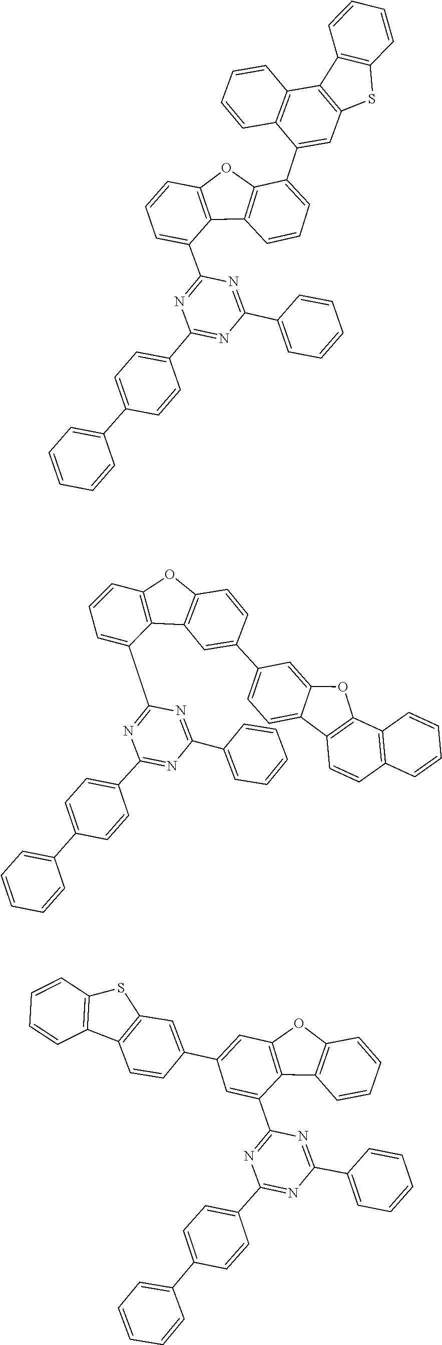 [SMILES: c1ccc(-c2ccc(-c3nc(-c4ccccc4)nc(-c4cccc5oc6c(-c7cc8sc9ccccc9c8c8ccccc78)cccc6c45)n3)cc2)cc1 ; c1ccc(-c2ccc(-c3nc(-c4ccccc4)nc(-c4cccc5oc6ccc(-c7ccc8c(c7)oc7c9ccccc9ccc87)cc6c45)n3)cc2)cc1 ; c1ccc(-c2ccc(-c3nc(-c4ccccc4)nc(-c4cc(-c5ccc6c(c5)sc5ccccc56)cc5oc6ccccc6c45)n3)cc2)cc1]

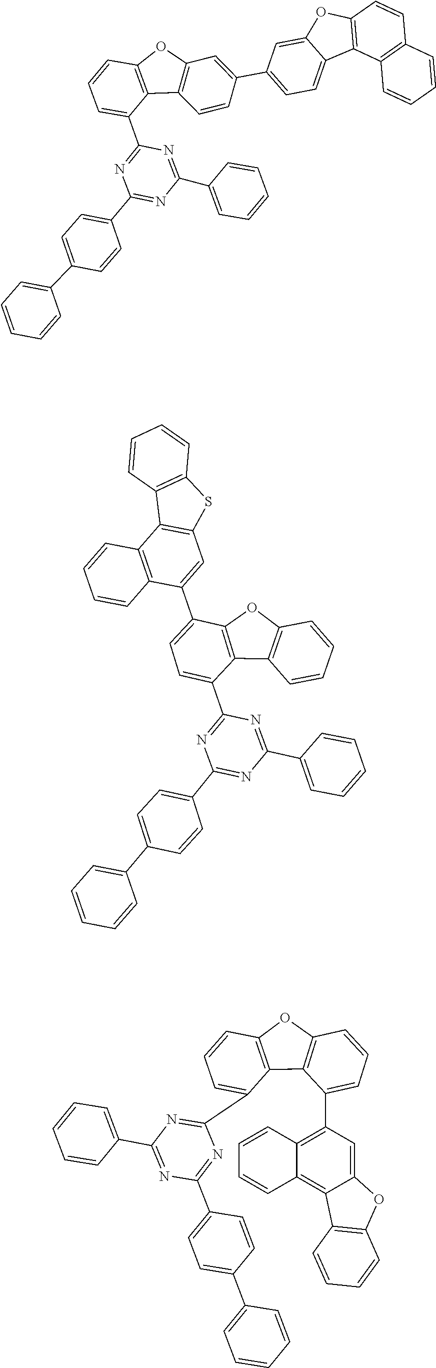 [SMILES: c1ccc(-c2ccc(-c3nc(-c4ccccc4)nc(-c4cccc5oc6cc(-c7ccc8c(c7)oc7ccc9ccccc9c78)ccc6c45)n3)cc2)cc1 ; c1ccc(-c2ccc(-c3nc(-c4ccccc4)nc(-c4ccc(-c5cc6sc7ccccc7c6c6ccccc56)c5oc6ccccc6c45)n3)cc2)cc1 ; c1ccc(-c2ccc(-c3nc(-c4ccccc4)nc(-c4cccc5oc6cccc(-c7cc8oc9ccccc9c8c8ccccc78)c6c45)n3)cc2)cc1]

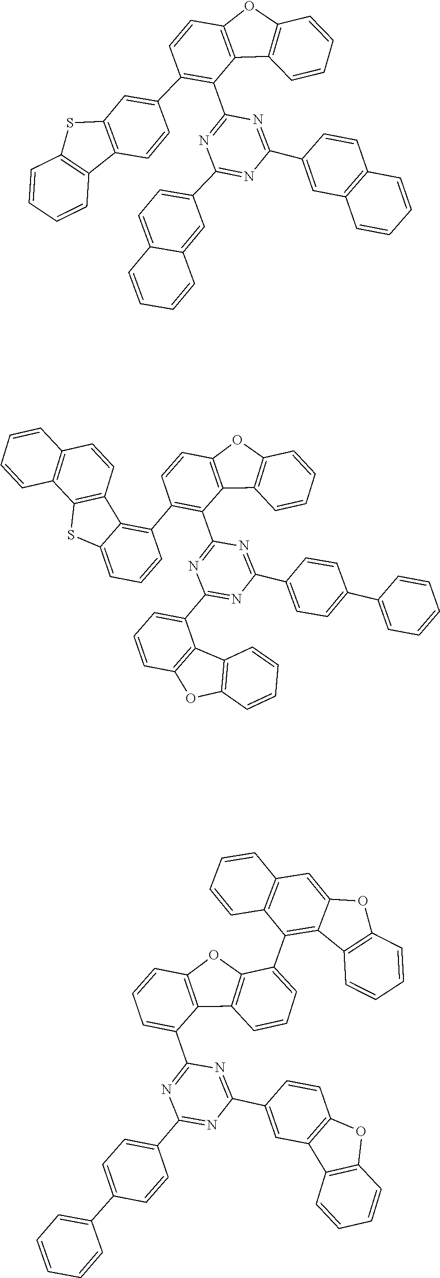 [SMILES: c1ccc2cc(-c3nc(-c4ccc5ccccc5c4)nc(-c4c(-c5ccc6c(c5)sc5ccccc56)ccc5oc6ccccc6c45)n3)ccc2c1 ; c1ccc(-c2ccc(-c3nc(-c4cccc5oc6ccccc6c45)nc(-c4c(-c5cccc6sc7c8ccccc8ccc7c56)ccc5oc6ccccc6c45)n3)cc2)cc1 ; c1ccc(-c2ccc(-c3nc(-c4ccc5oc6ccccc6c5c4)nc(-c4cccc5oc6c(-c7c8ccccc8cc8oc9ccccc9c78)cccc6c45)n3)cc2)cc1]

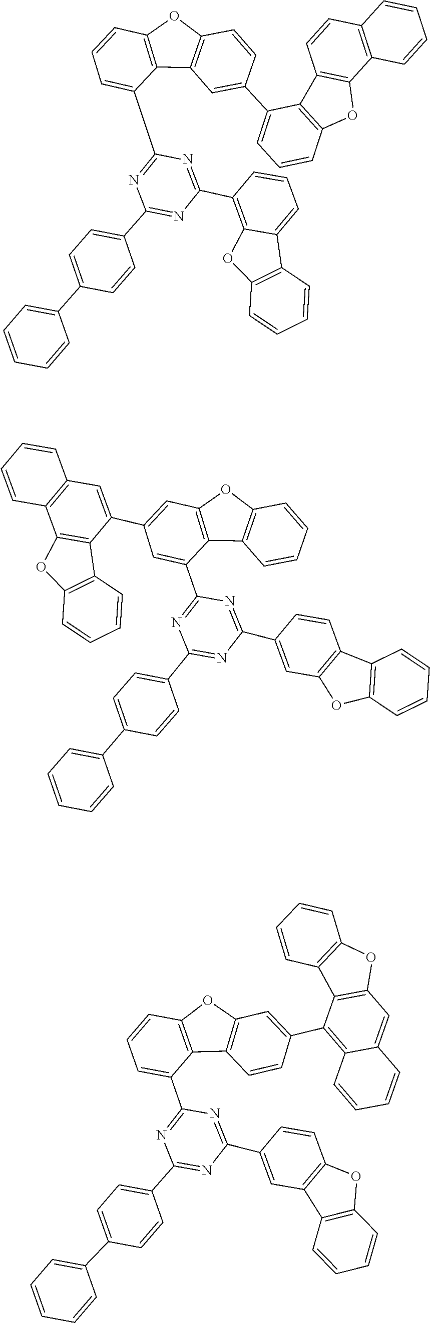 [SMILES: c1ccc(-c2ccc(-c3nc(-c4cccc5c4oc4ccccc45)nc(-c4cccc5oc6ccc(-c7cccc8oc9c%10ccccc%10ccc9c78)cc6c45)n3)cc2)cc1 ; c1ccc(-c2ccc(-c3nc(-c4ccc5c(c4)oc4ccccc45)nc(-c4cc(-c5cc6ccccc6c6oc7ccccc7c56)cc5oc6ccccc6c45)n3)cc2)cc1 ; c1ccc(-c2ccc(-c3nc(-c4ccc5oc6ccccc6c5c4)nc(-c4cccc5oc6cc(-c7c8ccccc8cc8oc9ccccc9c78)ccc6c45)n3)cc2)cc1]

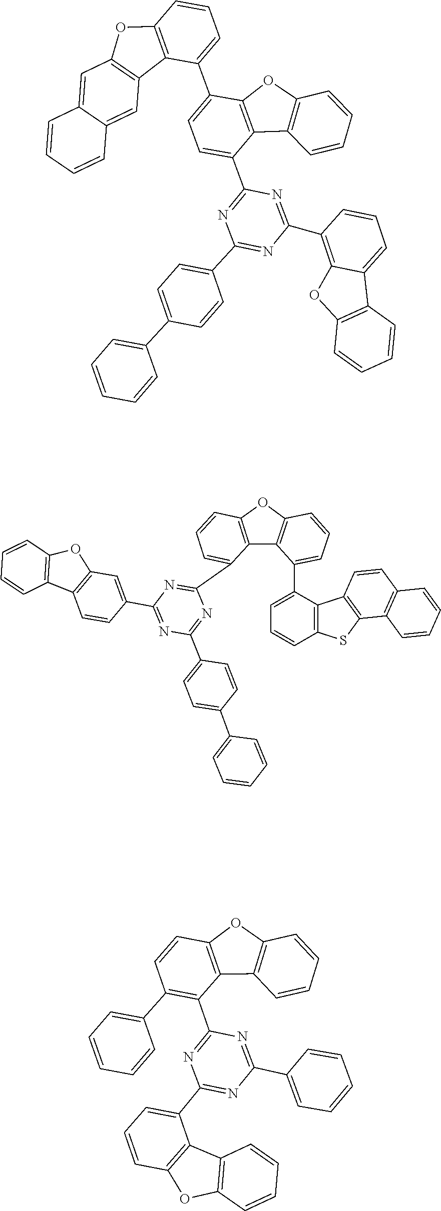 [SMILES: c1ccc(-c2ccc(-c3nc(-c4cccc5c4oc4ccccc45)nc(-c4ccc(-c5cccc6oc7cc8ccccc8cc7c56)c5oc6ccccc6c45)n3)cc2)cc1 ; c1ccc(-c2ccc(-c3nc(-c4ccc5c(c4)oc4ccccc45)nc(-c4cccc5oc6cccc(-c7cccc8sc9c%10ccccc%10ccc9c78)c6c45)n3)cc2)cc1 ; c1ccc(-c2nc(-c3cccc4oc5ccccc5c34)nc(-c3c(-c4ccccc4)ccc4oc5ccccc5c34)n2)cc1]

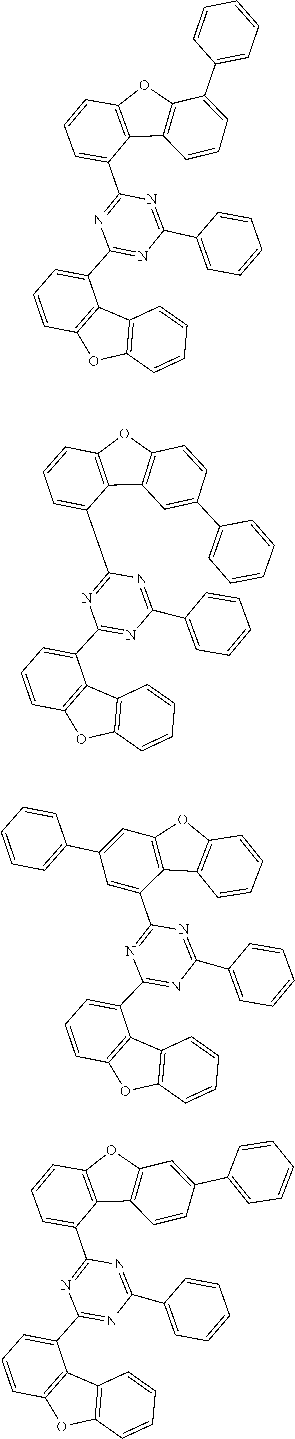 [SMILES: c1ccc(-c2nc(-c3cccc4oc5ccccc5c34)nc(-c3cccc4oc5c(-c6ccccc6)cccc5c34)n2)cc1 ; c1ccc(-c2ccc3oc4cccc(-c5nc(-c6ccccc6)nc(-c6cccc7oc8ccccc8c67)n5)c4c3c2)cc1 ; c1ccc(-c2cc(-c3nc(-c4ccccc4)nc(-c4cccc5oc6ccccc6c45)n3)c3c(c2)oc2ccccc23)cc1 ; c1ccc(-c2ccc3c(c2)oc2cccc(-c4nc(-c5ccccc5)nc(-c5cccc6oc7ccccc7c56)n4)c23)cc1]

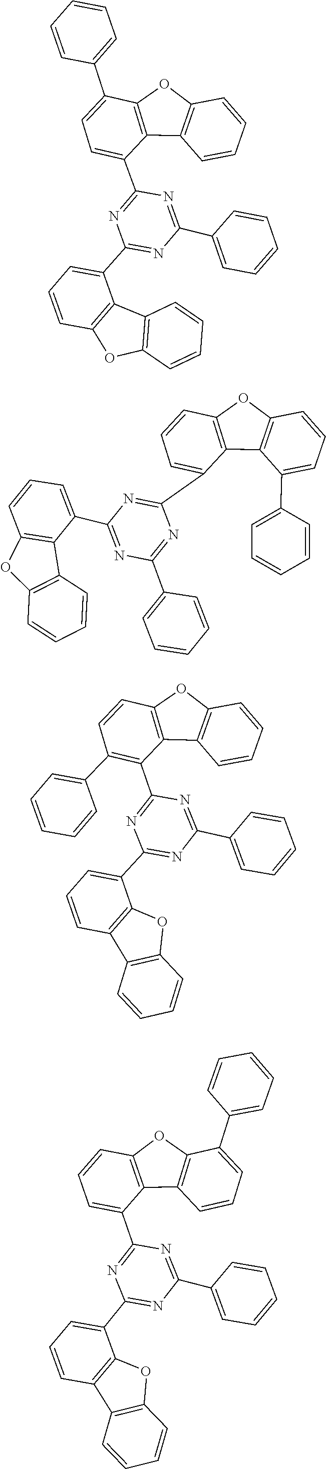

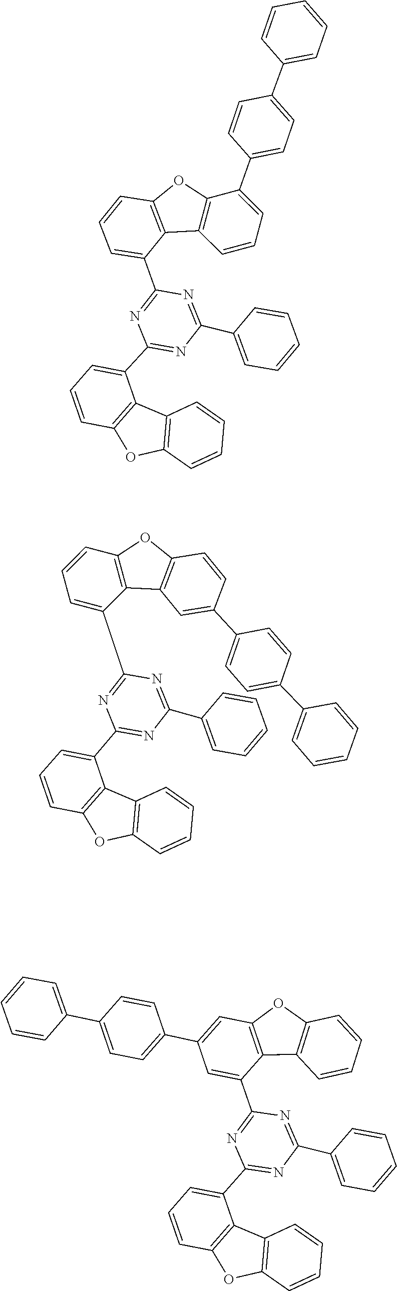 [SMILES: c1ccc(-c2ccc(-c3cccc4c3oc3cccc(-c5nc(-c6ccccc6)nc(-c6cccc7oc8ccccc8c67)n5)c34)cc2)cc1 ; c1ccc(-c2ccc(-c3ccc4oc5cccc(-c6nc(-c7ccccc7)nc(-c7cccc8oc9ccccc9c78)n6)c5c4c3)cc2)cc1 ; c1ccc(-c2ccc(-c3cc(-c4nc(-c5ccccc5)nc(-c5cccc6oc7ccccc7c56)n4)c4c(c3)oc3ccccc34)cc2)cc1]

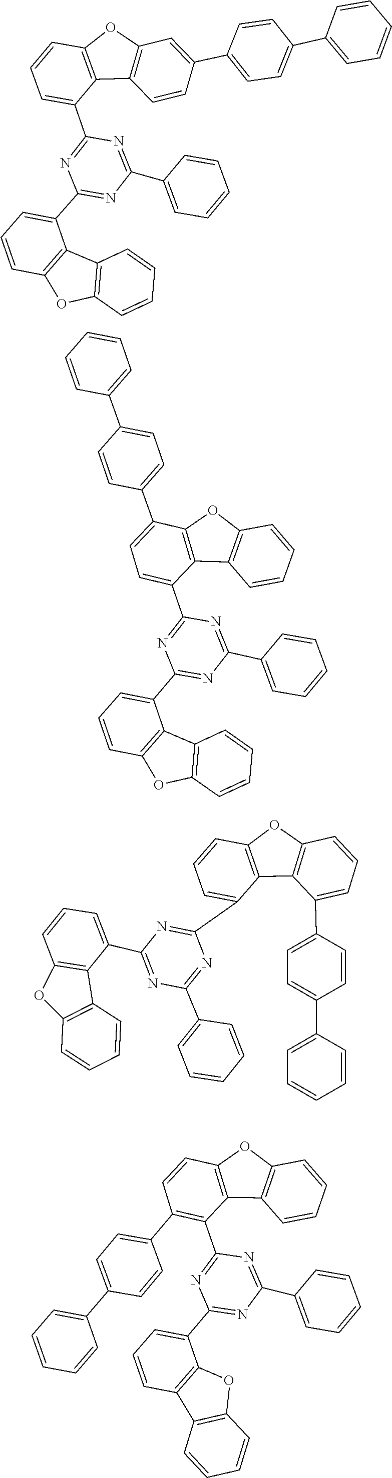

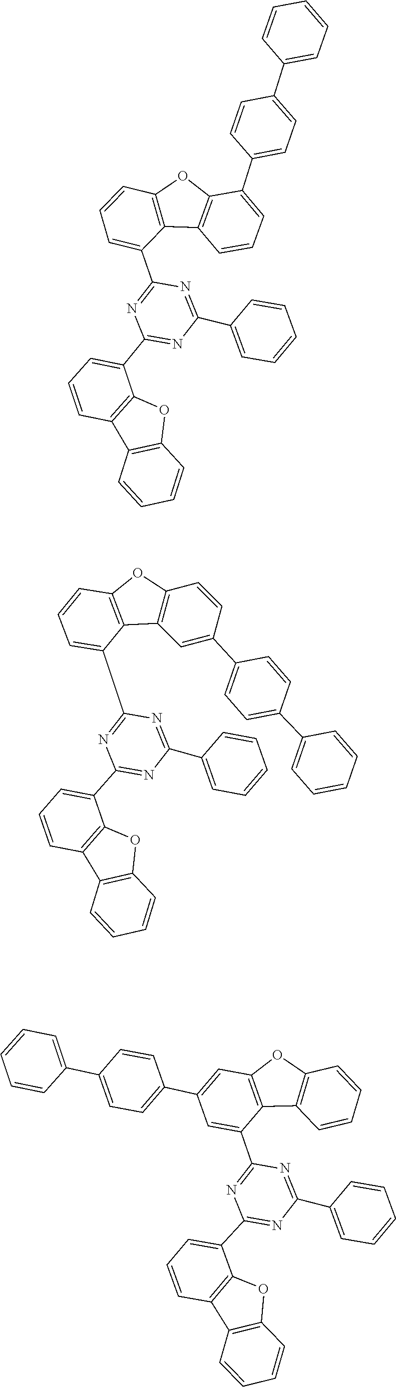 [SMILES: c1ccc(-c2ccc(-c3cccc4c3oc3cccc(-c5nc(-c6ccccc6)nc(-c6cccc7c6oc6ccccc67)n5)c34)cc2)cc1 ; c1ccc(-c2ccc(-c3ccc4oc5cccc(-c6nc(-c7ccccc7)nc(-c7cccc8c7oc7ccccc78)n6)c5c4c3)cc2)cc1 ; c1ccc(-c2ccc(-c3cc(-c4nc(-c5ccccc5)nc(-c5cccc6c5oc5ccccc56)n4)c4c(c3)oc3ccccc34)cc2)cc1]

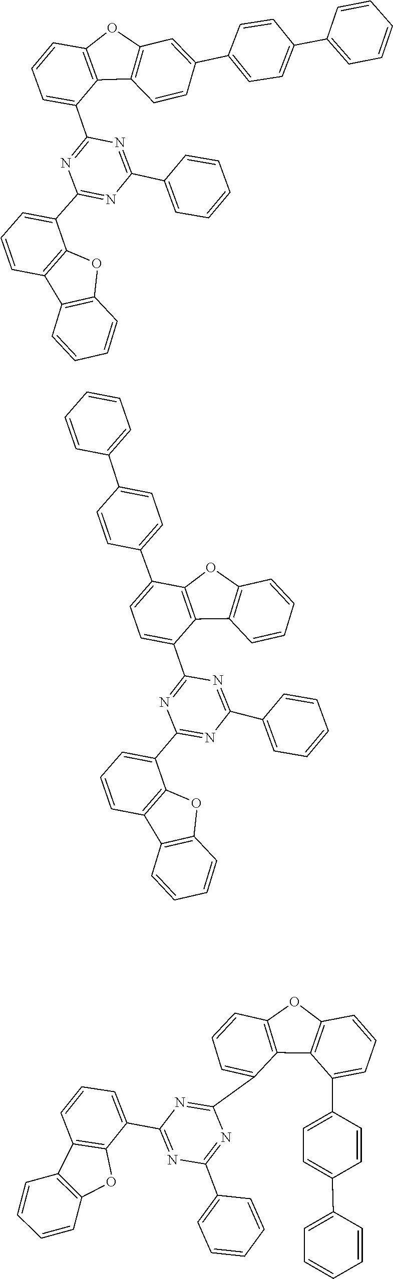

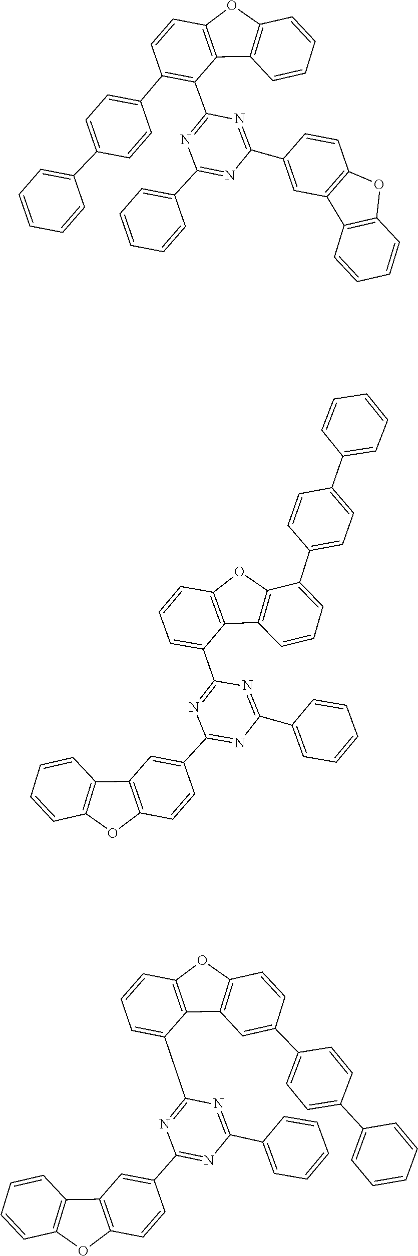 [SMILES: c1ccc(-c2ccc(-c3ccc4oc5ccccc5c4c3-c3nc(-c4ccccc4)nc(-c4ccc5oc6ccccc6c5c4)n3)cc2)cc1 ; c1ccc(-c2ccc(-c3cccc4c3oc3cccc(-c5nc(-c6ccccc6)nc(-c6ccc7oc8ccccc8c7c6)n5)c34)cc2)cc1 ; c1ccc(-c2ccc(-c3ccc4oc5cccc(-c6nc(-c7ccccc7)nc(-c7ccc8oc9ccccc9c8c7)n6)c5c4c3)cc2)cc1]

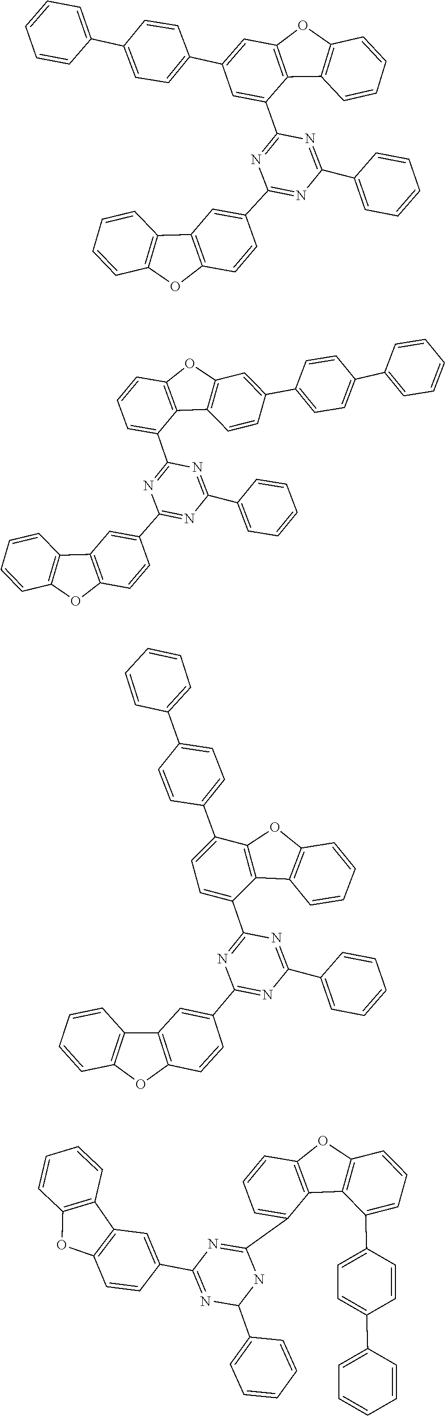

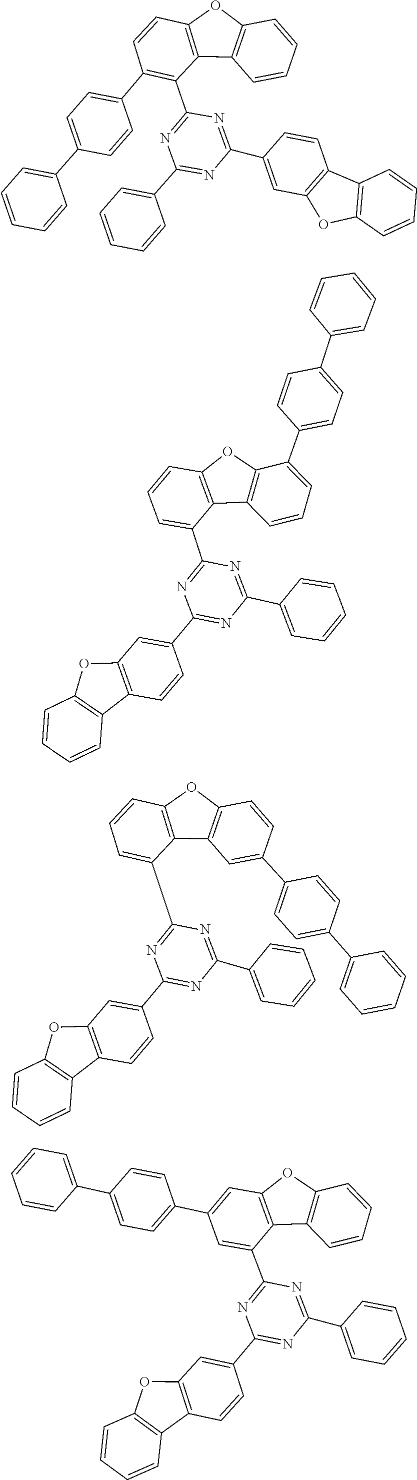

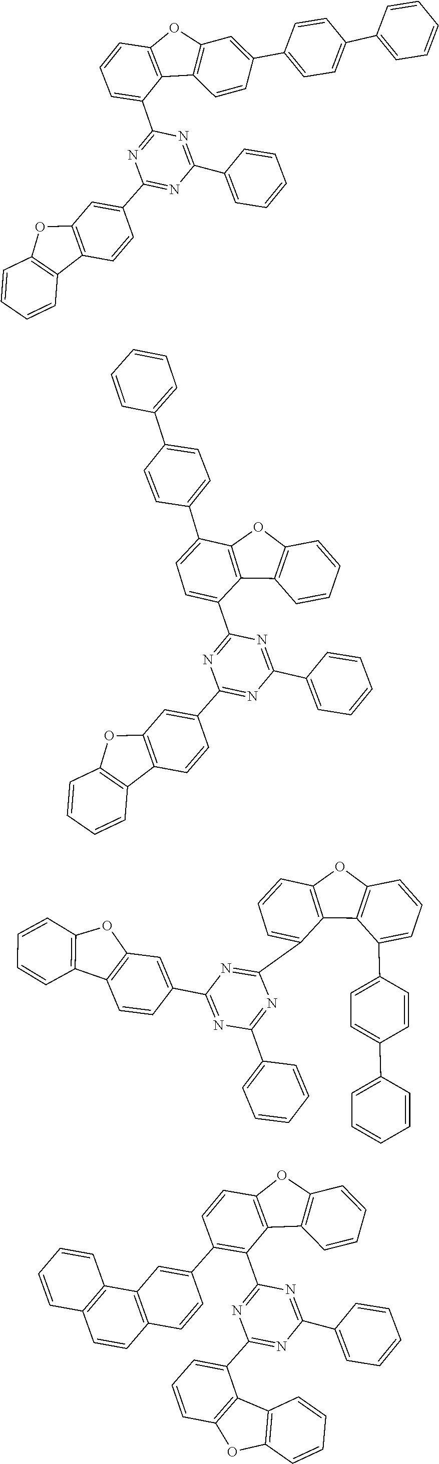 [SMILES: c1ccc(-c2ccc(-c3ccc4c(c3)oc3cccc(-c5nc(-c6ccccc6)nc(-c6ccc7c(c6)oc6ccccc67)n5)c34)cc2)cc1 ; c1ccc(-c2ccc(-c3ccc(-c4nc(-c5ccccc5)nc(-c5ccc6c(c5)oc5ccccc56)n4)c4c3oc3ccccc34)cc2)cc1 ; c1ccc(-c2ccc(-c3cccc4oc5cccc(-c6nc(-c7ccccc7)nc(-c7ccc8c(c7)oc7ccccc78)n6)c5c34)cc2)cc1 ; c1ccc(-c2nc(-c3cccc4oc5ccccc5c34)nc(-c3c(-c4ccc5ccc6ccccc6c5c4)ccc4oc5ccccc5c34)n2)cc1]

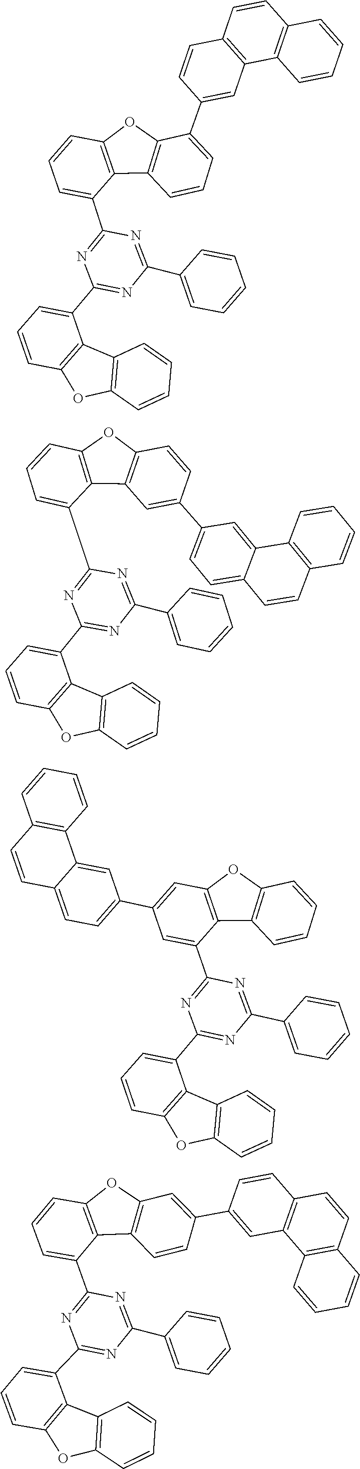

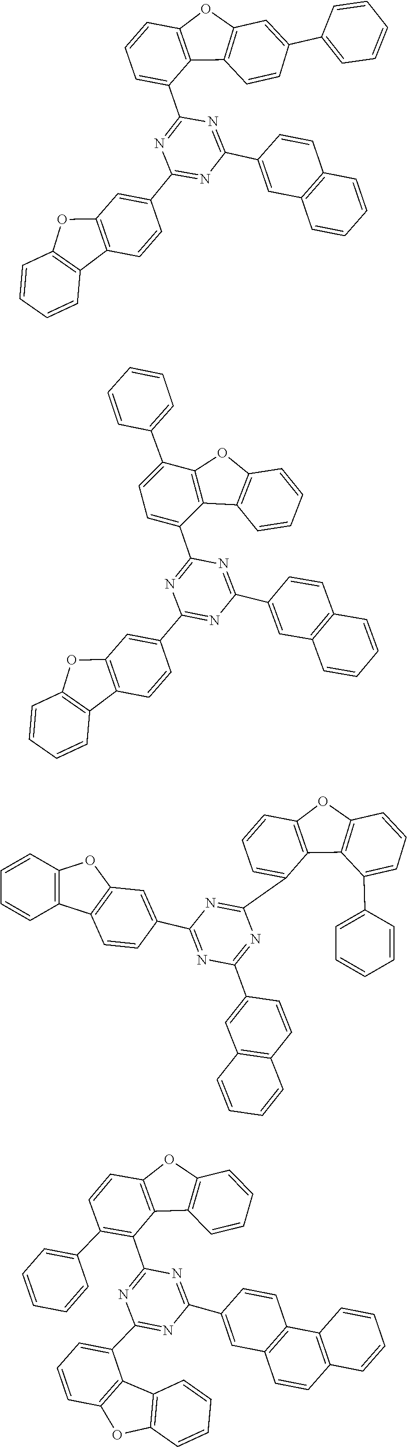 [SMILES: c1ccc(-c2ccc3c(c2)oc2cccc(-c4nc(-c5ccc6ccccc6c5)nc(-c5ccc6c(c5)oc5ccccc56)n4)c23)cc1 ; c1ccc(-c2ccc(-c3nc(-c4ccc5ccccc5c4)nc(-c4ccc5c(c4)oc4ccccc45)n3)c3c2oc2ccccc23)cc1 ; c1ccc(-c2cccc3oc4cccc(-c5nc(-c6ccc7ccccc7c6)nc(-c6ccc7c(c6)oc6ccccc67)n5)c4c23)cc1 ; c1ccc(-c2ccc3oc4ccccc4c3c2-c2nc(-c3ccc4c(ccc5ccccc54)c3)nc(-c3cccc4oc5ccccc5c34)n2)cc1]

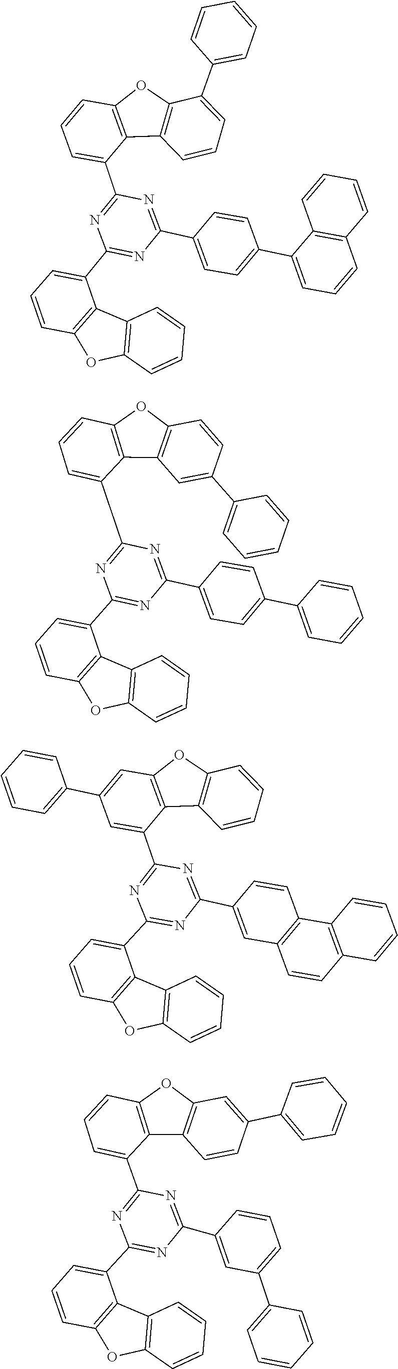

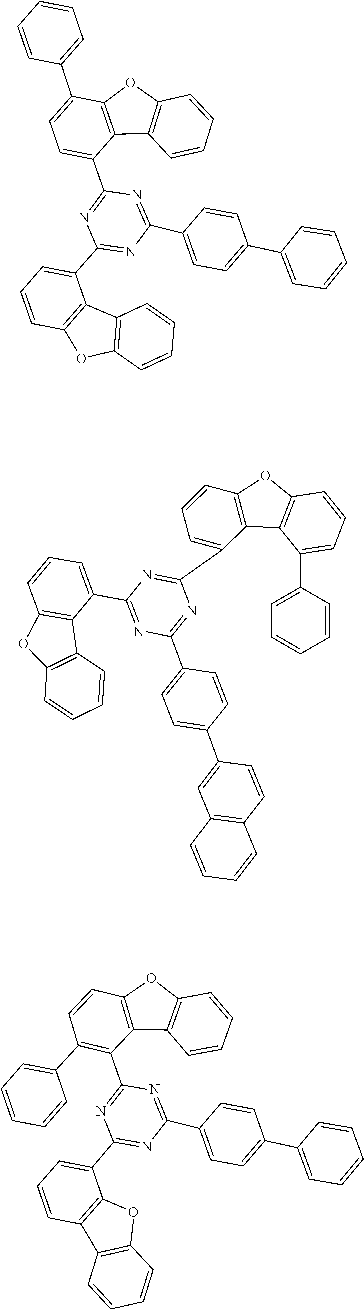 [SMILES: c1ccc(-c2ccc(-c3nc(-c4cccc5oc6ccccc6c45)nc(-c4ccc(-c5ccccc5)c5oc6ccccc6c45)n3)cc2)cc1 ; c1ccc(-c2cccc3oc4cccc(-c5nc(-c6ccc(-c7ccc8ccccc8c7)cc6)nc(-c6cccc7oc8ccccc8c67)n5)c4c23)cc1 ; c1ccc(-c2ccc(-c3nc(-c4cccc5c4oc4ccccc45)nc(-c4c(-c5ccccc5)ccc5oc6ccccc6c45)n3)cc2)cc1]

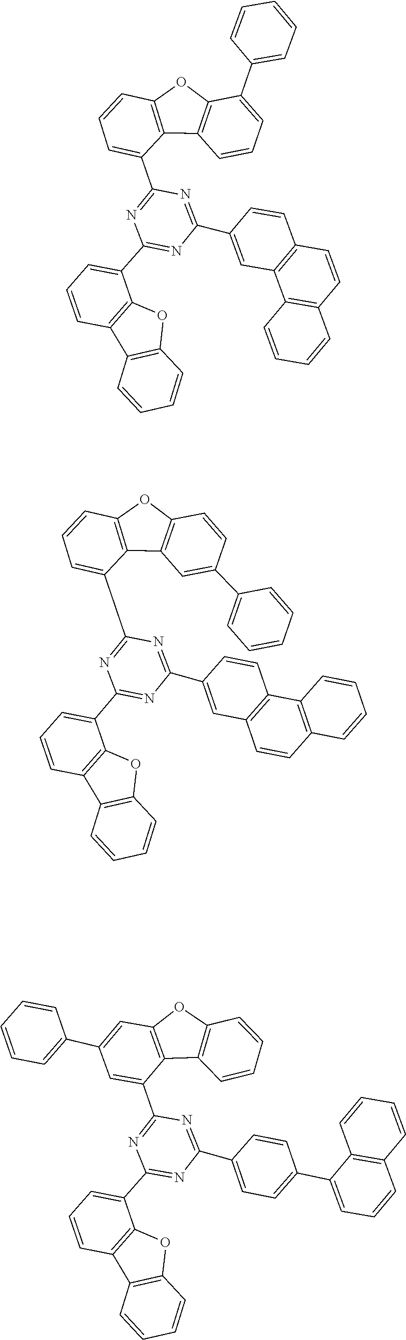 [SMILES: c1ccc(-c2cccc3c2oc2cccc(-c4nc(-c5ccc6ccc7ccccc7c6c5)nc(-c5cccc6c5oc5ccccc56)n4)c23)cc1 ; c1ccc(-c2ccc3oc4cccc(-c5nc(-c6ccc7c(ccc8ccccc87)c6)nc(-c6cccc7c6oc6ccccc67)n5)c4c3c2)cc1 ; c1ccc(-c2cc(-c3nc(-c4ccc(-c5cccc6ccccc56)cc4)nc(-c4cccc5c4oc4ccccc45)n3)c3c(c2)oc2ccccc23)cc1]

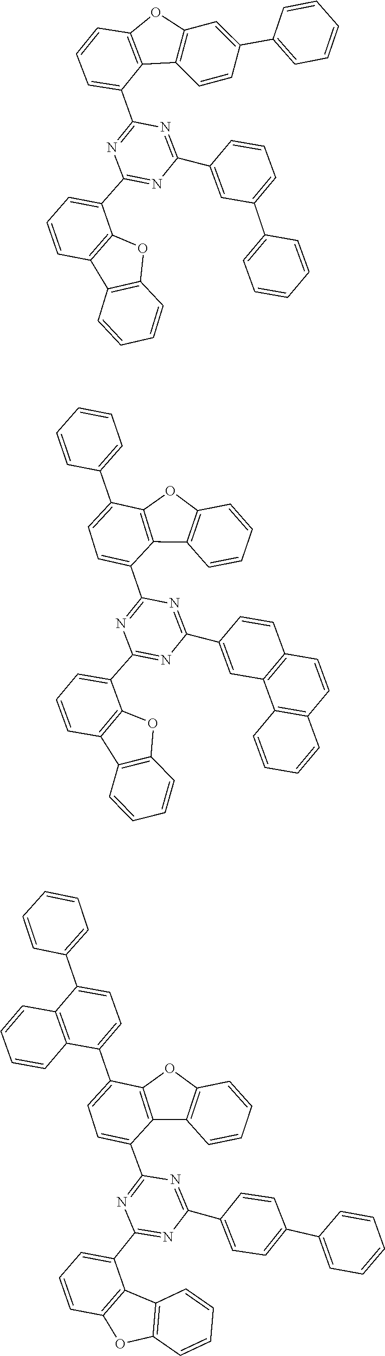 [SMILES: c1ccc(-c2cccc(-c3nc(-c4cccc5c4oc4ccccc45)nc(-c4cccc5oc6cc(-c7ccccc7)ccc6c45)n3)c2)cc1 ; c1ccc(-c2ccc(-c3nc(-c4ccc5ccc6ccccc6c5c4)nc(-c4cccc5c4oc4ccccc45)n3)c3c2oc2ccccc23)cc1 ; c1ccc(-c2ccc(-c3nc(-c4cccc5oc6ccccc6c45)nc(-c4ccc(-c5ccc(-c6ccccc6)c6ccccc56)c5oc6ccccc6c45)n3)cc2)cc1]

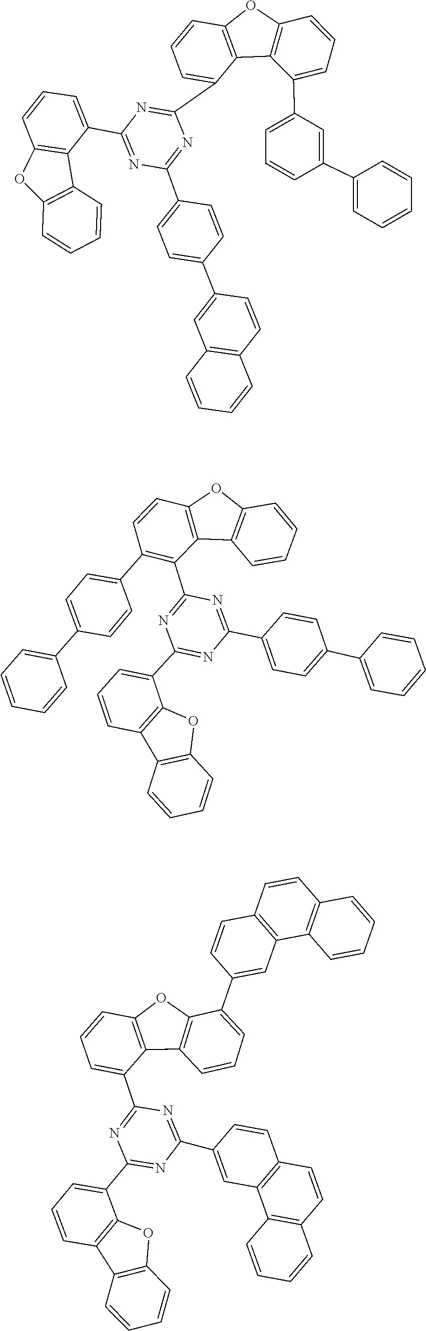 [SMILES: c1ccc(-c2cccc(-c3cccc4oc5cccc(-c6nc(-c7ccc(-c8ccc9ccccc9c8)cc7)nc(-c7cccc8oc9ccccc9c78)n6)c5c34)c2)cc1 ; c1ccc(-c2ccc(-c3nc(-c4cccc5c4oc4ccccc45)nc(-c4c(-c5ccc(-c6ccccc6)cc5)ccc5oc6ccccc6c45)n3)cc2)cc1 ; c1ccc2c(c1)ccc1ccc(-c3nc(-c4cccc5c4oc4ccccc45)nc(-c4cccc5oc6c(-c7ccc8ccc9ccccc9c8c7)cccc6c45)n3)cc12]

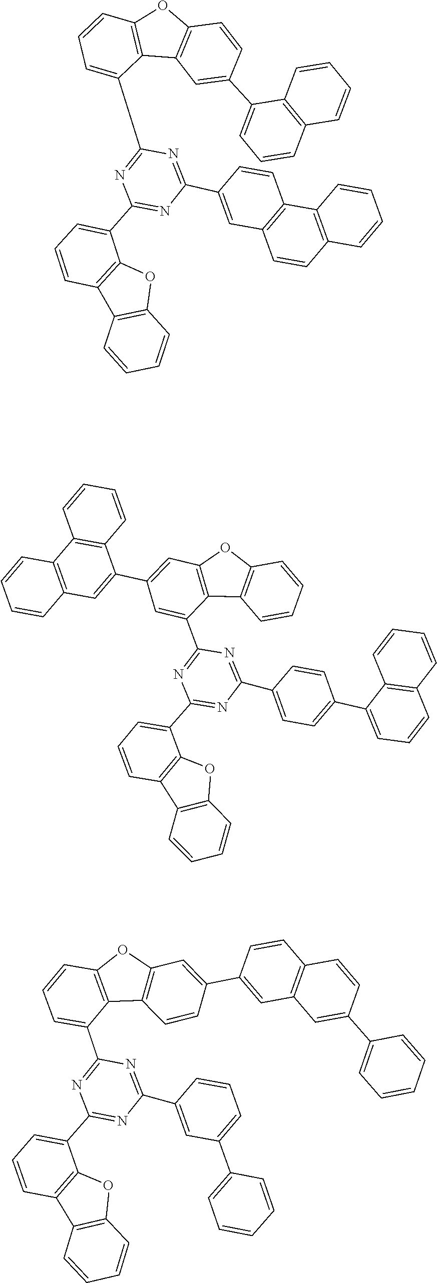 [SMILES: c1ccc2c(-c3ccc4oc5cccc(-c6nc(-c7ccc8c(ccc9ccccc98)c7)nc(-c7cccc8c7oc7ccccc78)n6)c5c4c3)cccc2c1 ; c1ccc2c(-c3ccc(-c4nc(-c5cccc6c5oc5ccccc56)nc(-c5cc(-c6cc7ccccc7c7ccccc67)cc6oc7ccccc7c56)n4)cc3)cccc2c1 ; c1ccc(-c2cccc(-c3nc(-c4cccc5c4oc4ccccc45)nc(-c4cccc5oc6cc(-c7ccc8ccc(-c9ccccc9)cc8c7)ccc6c45)n3)c2)cc1]

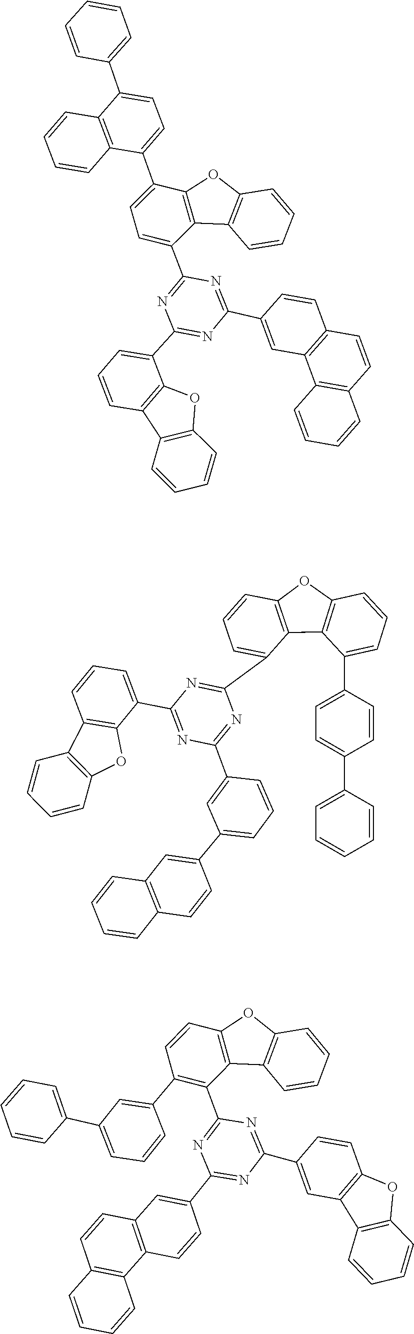 [SMILES: c1ccc(-c2ccc(-c3ccc(-c4nc(-c5ccc6ccc7ccccc7c6c5)nc(-c5cccc6c5oc5ccccc56)n4)c4c3oc3ccccc34)c3ccccc23)cc1 ; c1ccc(-c2ccc(-c3cccc4oc5cccc(-c6nc(-c7cccc(-c8ccc9ccccc9c8)c7)nc(-c7cccc8c7oc7ccccc78)n6)c5c34)cc2)cc1 ; c1ccc(-c2cccc(-c3ccc4oc5ccccc5c4c3-c3nc(-c4ccc5c(ccc6ccccc65)c4)nc(-c4ccc5oc6ccccc6c5c4)n3)c2)cc1]

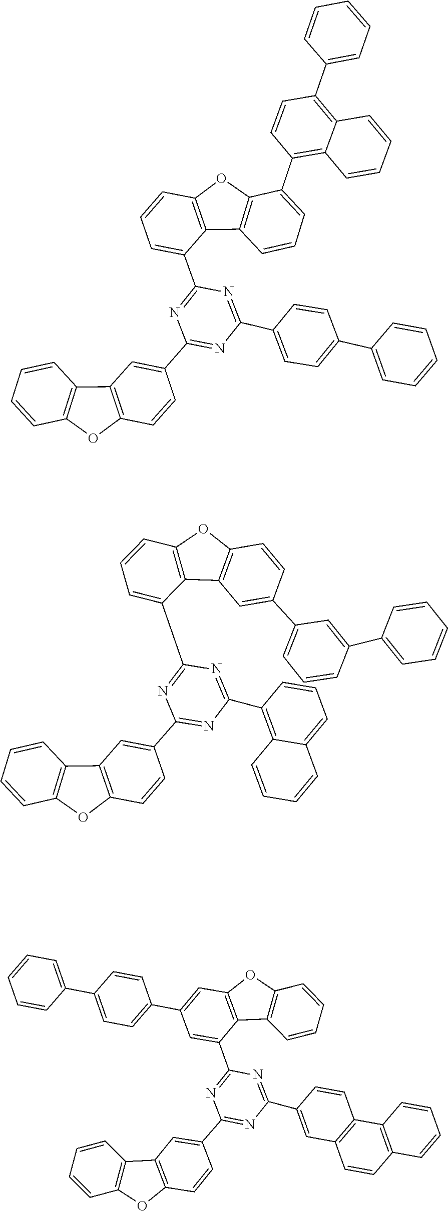 [SMILES: c1ccc(-c2ccc(-c3nc(-c4ccc5oc6ccccc6c5c4)nc(-c4cccc5oc6c(-c7ccc(-c8ccccc8)c8ccccc78)cccc6c45)n3)cc2)cc1 ; c1ccc(-c2cccc(-c3ccc4oc5cccc(-c6nc(-c7ccc8oc9ccccc9c8c7)nc(-c7cccc8ccccc78)n6)c5c4c3)c2)cc1 ; c1ccc(-c2ccc(-c3cc(-c4nc(-c5ccc6c(ccc7ccccc76)c5)nc(-c5ccc6oc7ccccc7c6c5)n4)c4c(c3)oc3ccccc34)cc2)cc1]

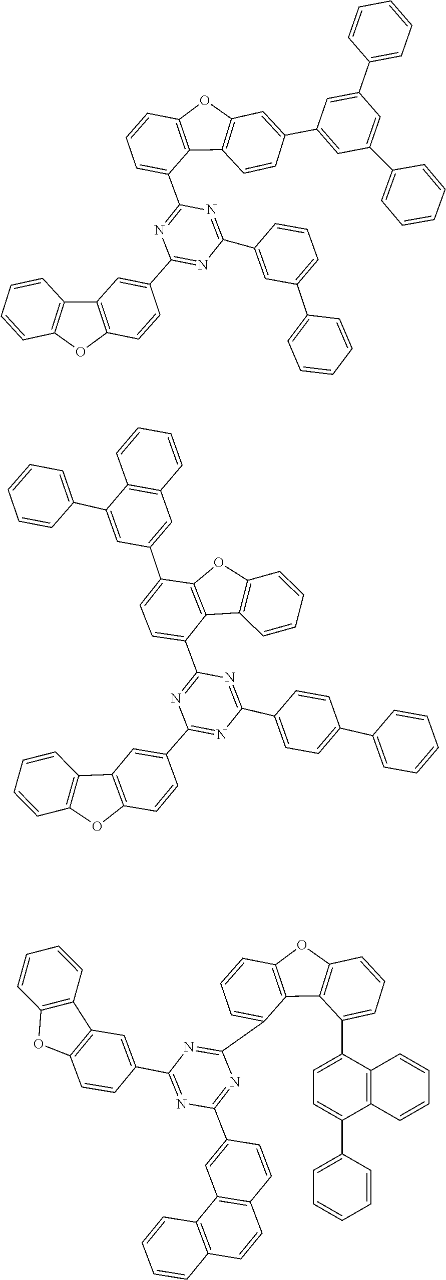 [SMILES: c1ccc(-c2cc(-c3ccccc3)cc(-c3ccc4c(c3)oc3cccc(-c5nc(-c6cccc(-c7ccccc7)c6)nc(-c6ccc7oc8ccccc8c7c6)n5)c34)c2)cc1 ; c1ccc(-c2ccc(-c3nc(-c4ccc5oc6ccccc6c5c4)nc(-c4ccc(-c5cc(-c6ccccc6)c6ccccc6c5)c5oc6ccccc6c45)n3)cc2)cc1 ; c1ccc(-c2ccc(-c3cccc4oc5cccc(-c6nc(-c7ccc8ccc9ccccc9c8c7)nc(-c7ccc8oc9ccccc9c8c7)n6)c5c34)c3ccccc23)cc1]

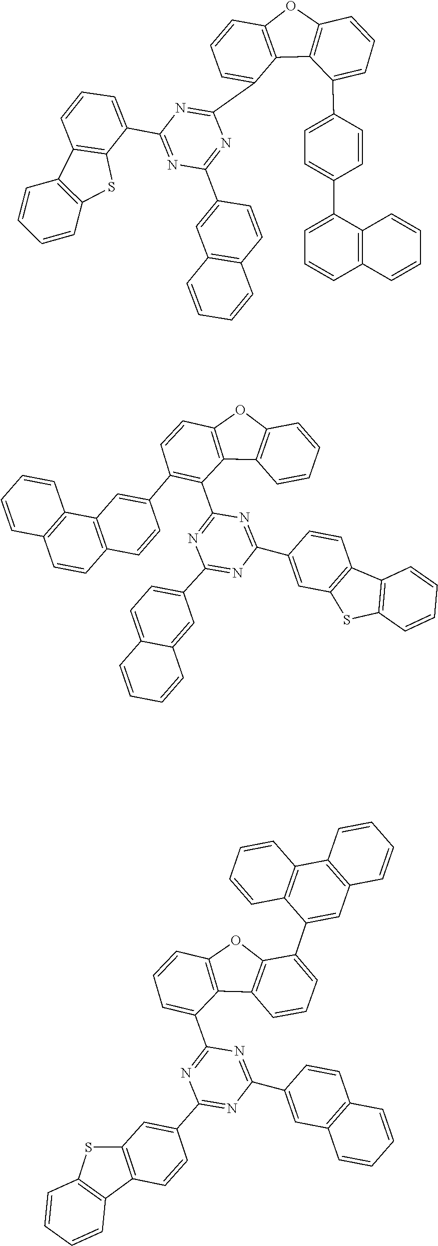 [SMILES: c1ccc2cc(-c3nc(-c4cccc5c4sc4ccccc45)nc(-c4cccc5oc6cccc(-c7ccc(-c8cccc9ccccc89)cc7)c6c45)n3)ccc2c1 ; c1ccc2cc(-c3nc(-c4ccc5c(c4)sc4ccccc45)nc(-c4c(-c5ccc6ccc7ccccc7c6c5)ccc5oc6ccccc6c45)n3)ccc2c1 ; c1ccc2cc(-c3nc(-c4ccc5c(c4)sc4ccccc45)nc(-c4cccc5oc6c(-c7cc8ccccc8c8ccccc78)cccc6c45)n3)ccc2c1]

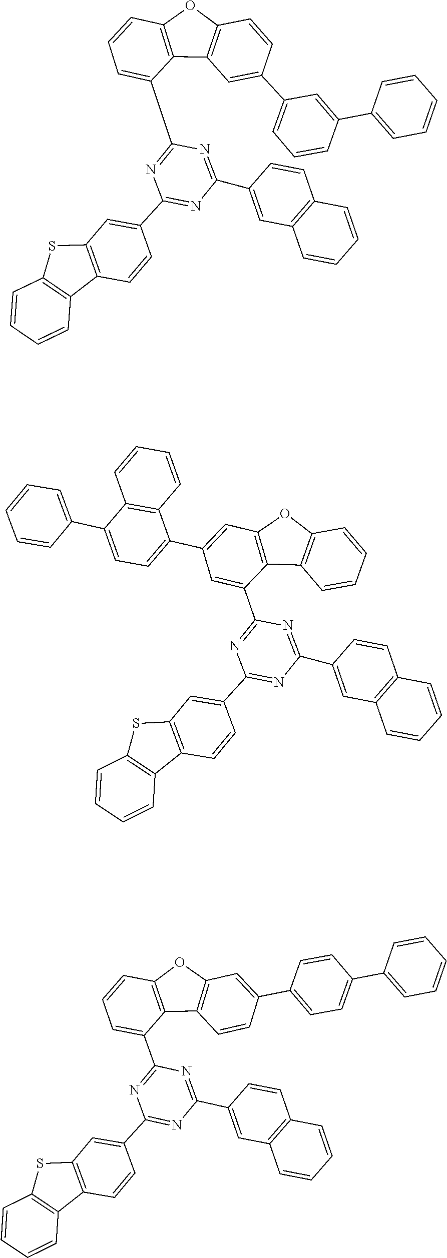 [SMILES: c1ccc(-c2cccc(-c3ccc4oc5cccc(-c6nc(-c7ccc8ccccc8c7)nc(-c7ccc8c(c7)sc7ccccc78)n6)c5c4c3)c2)cc1 ; c1ccc(-c2ccc(-c3cc(-c4nc(-c5ccc6ccccc6c5)nc(-c5ccc6c(c5)sc5ccccc56)n4)c4c(c3)oc3ccccc34)c3ccccc23)cc1 ; c1ccc(-c2ccc(-c3ccc4c(c3)oc3cccc(-c5nc(-c6ccc7ccccc7c6)nc(-c6ccc7c(c6)sc6ccccc67)n5)c34)cc2)cc1]

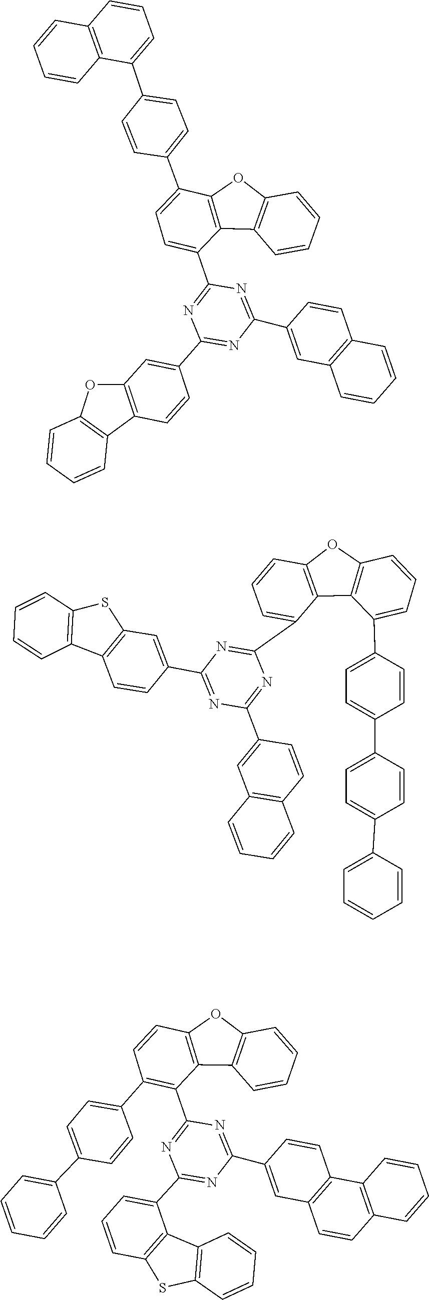 [SMILES: c1ccc2cc(-c3nc(-c4ccc5c(c4)oc4ccccc45)nc(-c4ccc(-c5ccc(-c6cccc7ccccc67)cc5)c5oc6ccccc6c45)n3)ccc2c1 ; c1ccc(-c2ccc(-c3ccc(-c4cccc5oc6cccc(-c7nc(-c8ccc9ccccc9c8)nc(-c8ccc9c(c8)sc8ccccc89)n7)c6c45)cc3)cc2)cc1 ; c1ccc(-c2ccc(-c3ccc4oc5ccccc5c4c3-c3nc(-c4ccc5c(ccc6ccccc65)c4)nc(-c4cccc5sc6ccccc6c45)n3)cc2)cc1]

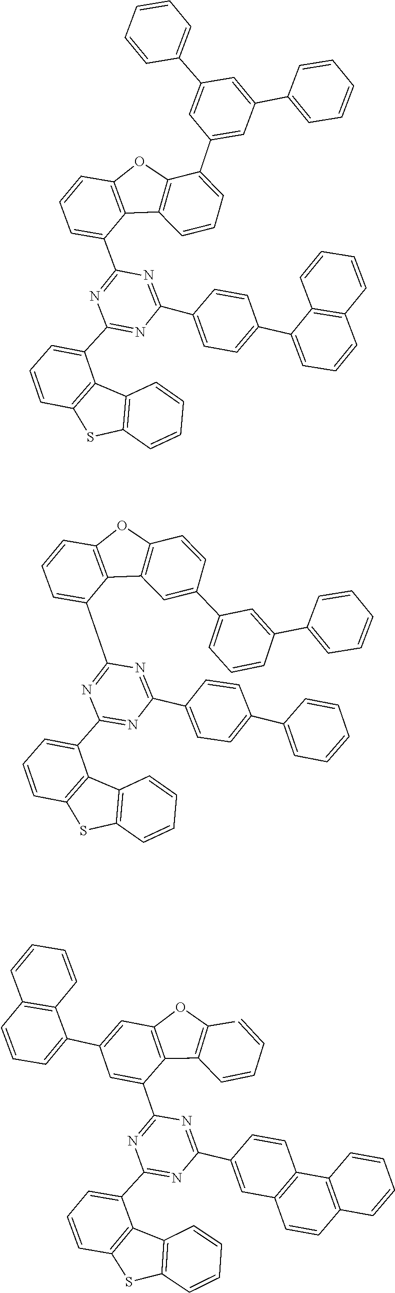 [SMILES: c1ccc(-c2cc(-c3ccccc3)cc(-c3cccc4c3oc3cccc(-c5nc(-c6ccc(-c7cccc8ccccc78)cc6)nc(-c6cccc7sc8ccccc8c67)n5)c34)c2)cc1 ; c1ccc(-c2ccc(-c3nc(-c4cccc5oc6ccc(-c7cccc(-c8ccccc8)c7)cc6c45)nc(-c4cccc5sc6ccccc6c45)n3)cc2)cc1 ; C=C/C=C\c1c(CC)oc2cc(-c3cccc4ccccc34)cc(-c3nc(-c4ccc5c(ccc6ccccc65)c4)nc(-c4cccc5sc6ccccc6c45)n3)c12]

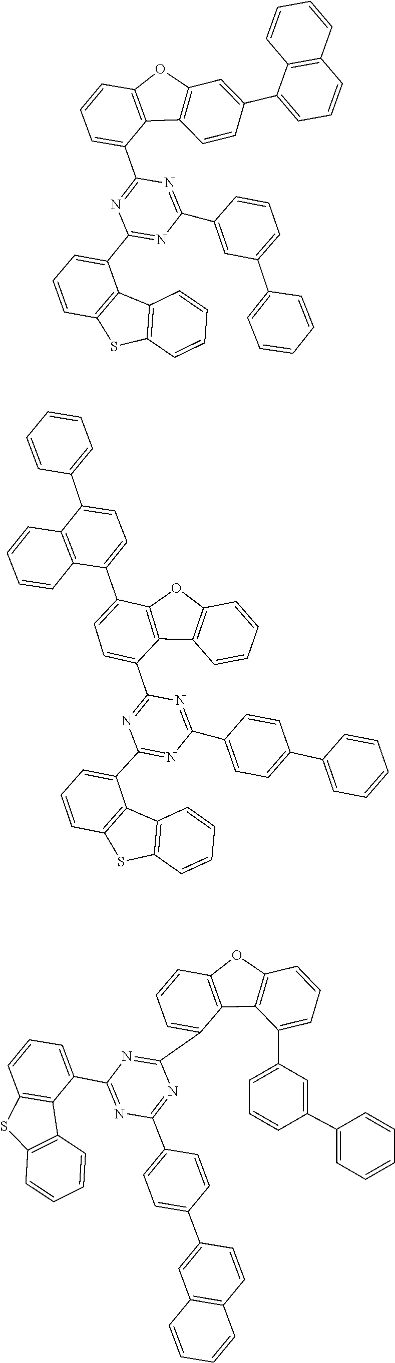 [SMILES: c1ccc(-c2cccc(-c3nc(-c4cccc5oc6cc(-c7cccc8ccccc78)ccc6c45)nc(-c4cccc5sc6ccccc6c45)n3)c2)cc1 ; c1ccc(-c2ccc(-c3nc(-c4cccc5sc6ccccc6c45)nc(-c4ccc(-c5ccc(-c6ccccc6)c6ccccc56)c5oc6ccccc6c45)n3)cc2)cc1 ; c1ccc(-c2cccc(-c3cccc4oc5cccc(-c6nc(-c7ccc(-c8ccc9ccccc9c8)cc7)nc(-c7cccc8sc9ccccc9c78)n6)c5c34)c2)cc1]

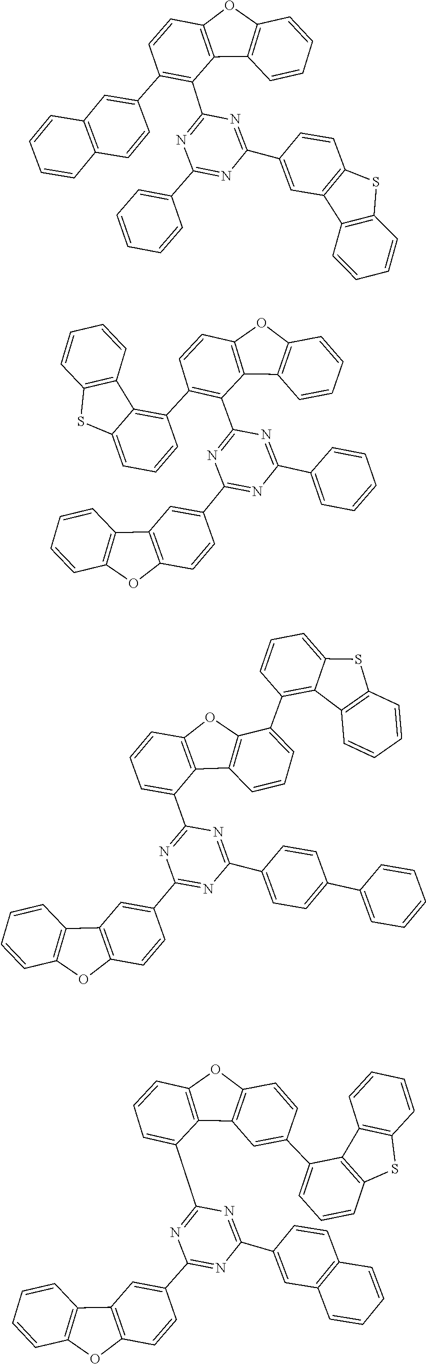 [SMILES: c1ccc(-c2nc(-c3ccc4sc5ccccc5c4c3)nc(-c3c(-c4ccc5ccccc5c4)ccc4oc5ccccc5c34)n2)cc1 ; c1ccc(-c2nc(-c3ccc4oc5ccccc5c4c3)nc(-c3c(-c4cccc5sc6ccccc6c45)ccc4oc5ccccc5c34)n2)cc1 ; c1ccc(-c2ccc(-c3nc(-c4ccc5oc6ccccc6c5c4)nc(-c4cccc5oc6c(-c7cccc8sc9ccccc9c78)cccc6c45)n3)cc2)cc1 ; c1ccc2cc(-c3nc(-c4ccc5oc6ccccc6c5c4)nc(-c4cccc5oc6ccc(-c7cccc8sc9ccccc9c78)cc6c45)n3)ccc2c1]

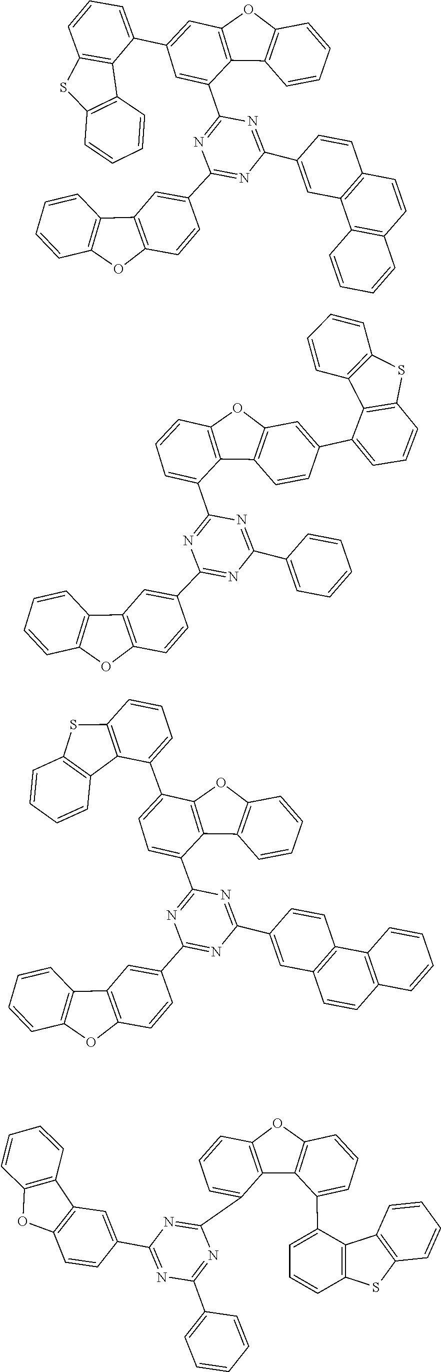

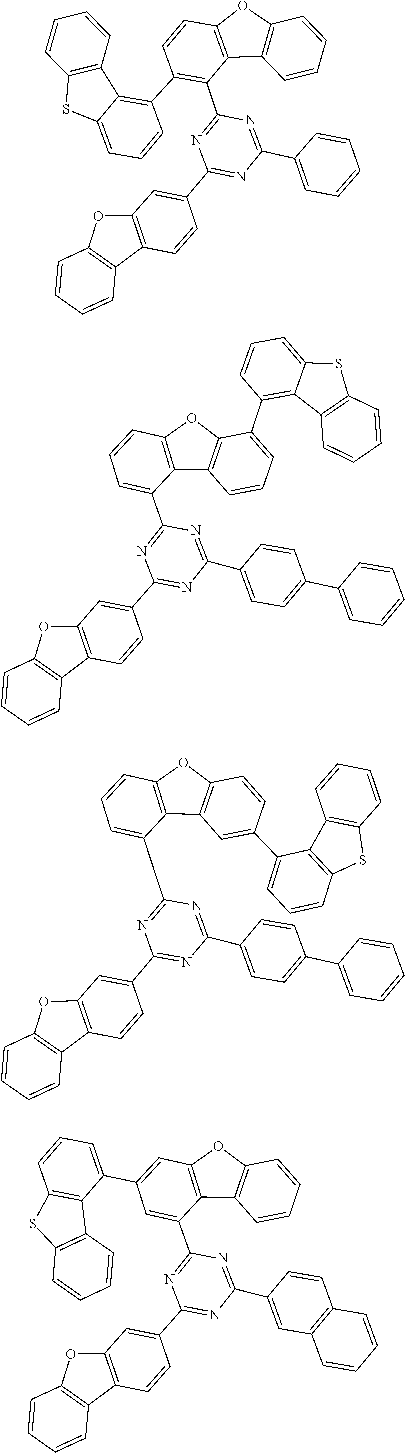 [SMILES: c1ccc(-c2nc(-c3ccc4c(c3)oc3ccccc34)nc(-c3c(-c4cccc5sc6ccccc6c45)ccc4oc5ccccc5c34)n2)cc1 ; c1ccc(-c2ccc(-c3nc(-c4ccc5c(c4)oc4ccccc45)nc(-c4cccc5oc6c(-c7cccc8sc9ccccc9c78)cccc6c45)n3)cc2)cc1 ; c1ccc(-c2ccc(-c3nc(-c4ccc5c(c4)oc4ccccc45)nc(-c4cccc5oc6ccc(-c7cccc8sc9ccccc9c78)cc6c45)n3)cc2)cc1 ; c1ccc2cc(-c3nc(-c4ccc5c(c4)oc4ccccc45)nc(-c4cc(-c5cccc6sc7ccccc7c56)cc5oc6ccccc6c45)n3)ccc2c1]

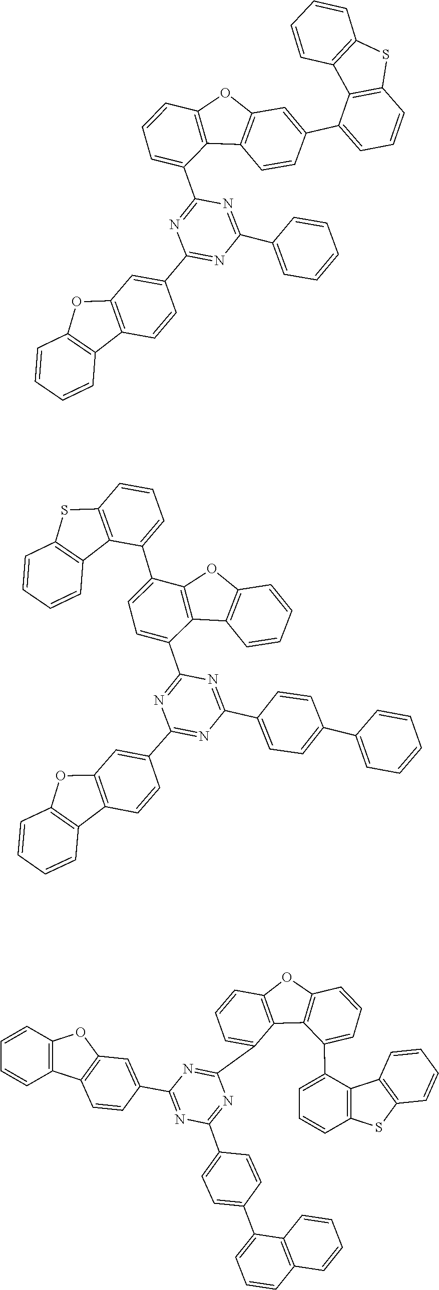 [SMILES: c1ccc(-c2nc(-c3ccc4c(c3)oc3ccccc34)nc(-c3cccc4oc5cc(-c6cccc7sc8ccccc8c67)ccc5c34)n2)cc1 ; c1ccc(-c2ccc(-c3nc(-c4ccc5c(c4)oc4ccccc45)nc(-c4ccc(-c5cccc6sc7ccccc7c56)c5oc6ccccc6c45)n3)cc2)cc1 ; c1ccc2c(-c3ccc(-c4nc(-c5ccc6c(c5)oc5ccccc56)nc(-c5cccc6oc7cccc(-c8cccc9sc%10ccccc%10c89)c7c56)n4)cc3)cccc2c1]

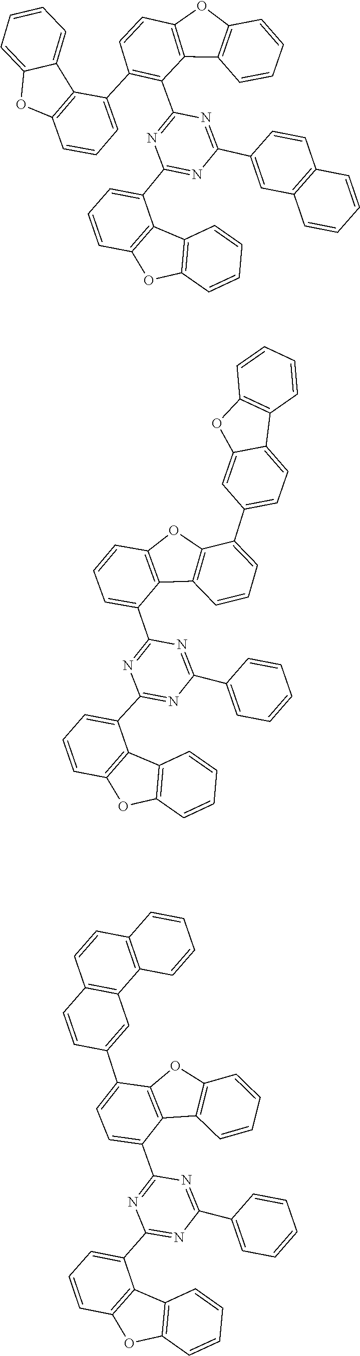 [SMILES: c1ccc2cc(-c3nc(-c4cccc5oc6ccccc6c45)nc(-c4c(-c5cccc6oc7ccccc7c56)ccc5oc6ccccc6c45)n3)ccc2c1 ; c1ccc(-c2nc(-c3cccc4oc5ccccc5c34)nc(-c3cccc4oc5c(-c6ccc7c(c6)oc6ccccc67)cccc5c34)n2)cc1 ; c1ccc(-c2nc(-c3cccc4oc5ccccc5c34)nc(-c3ccc(-c4ccc5ccc6ccccc6c5c4)c4oc5ccccc5c34)n2)cc1]

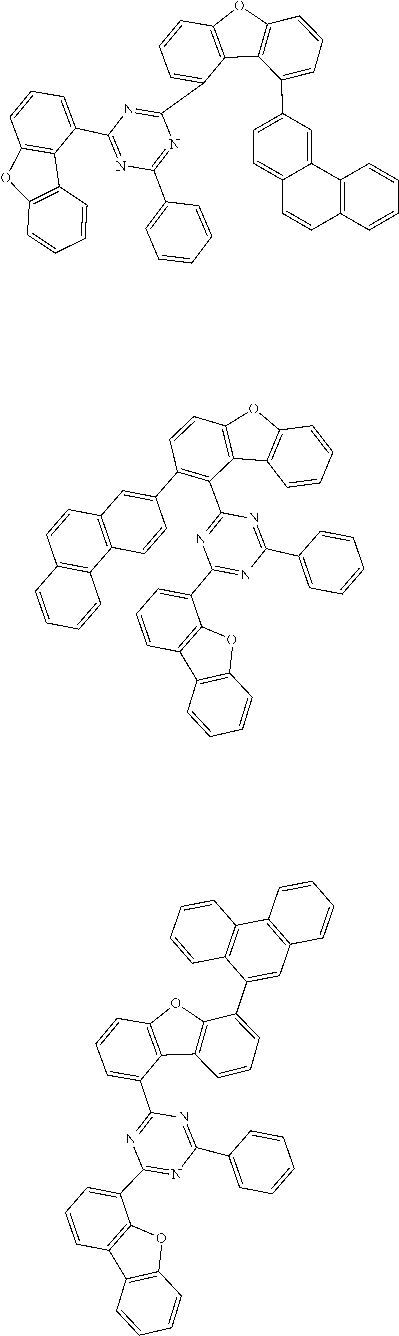 [SMILES: c1ccc(-c2nc(-c3cccc4oc5ccccc5c34)nc(-c3cccc4oc5cccc(-c6ccc7ccc8ccccc8c7c6)c5c34)n2)cc1 ; c1ccc(-c2nc(-c3cccc4c3oc3ccccc34)nc(-c3c(-c4ccc5c(ccc6ccccc65)c4)ccc4oc5ccccc5c34)n2)cc1 ; c1ccc(-c2nc(-c3cccc4c3oc3ccccc34)nc(-c3cccc4oc5c(-c6cc7ccccc7c7ccccc67)cccc5c34)n2)cc1]

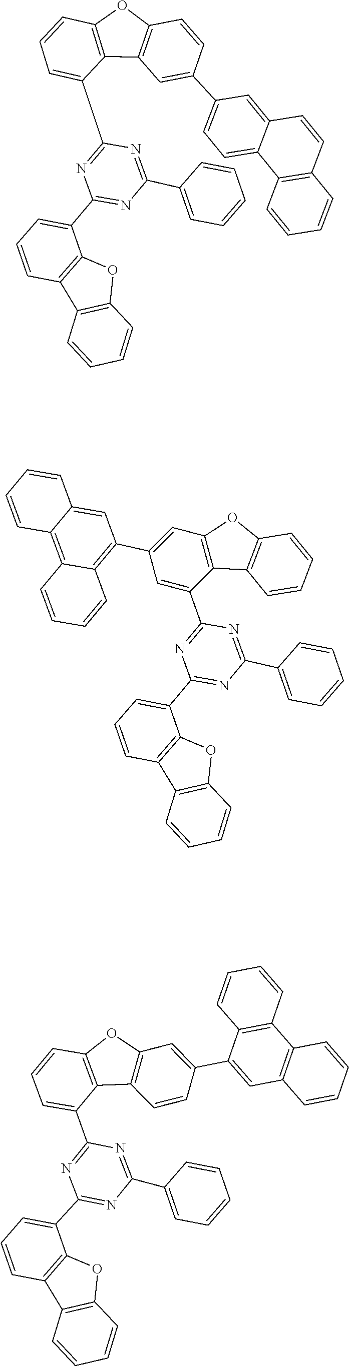 [SMILES: c1ccc(-c2nc(-c3cccc4c3oc3ccccc34)nc(-c3cccc4oc5ccc(-c6ccc7c(ccc8ccccc87)c6)cc5c34)n2)cc1 ; c1ccc(-c2nc(-c3cccc4c3oc3ccccc34)nc(-c3cc(-c4cc5ccccc5c5ccccc45)cc4oc5ccccc5c34)n2)cc1 ; c1ccc(-c2nc(-c3cccc4c3oc3ccccc34)nc(-c3cccc4oc5cc(-c6cc7ccccc7c7ccccc67)ccc5c34)n2)cc1]

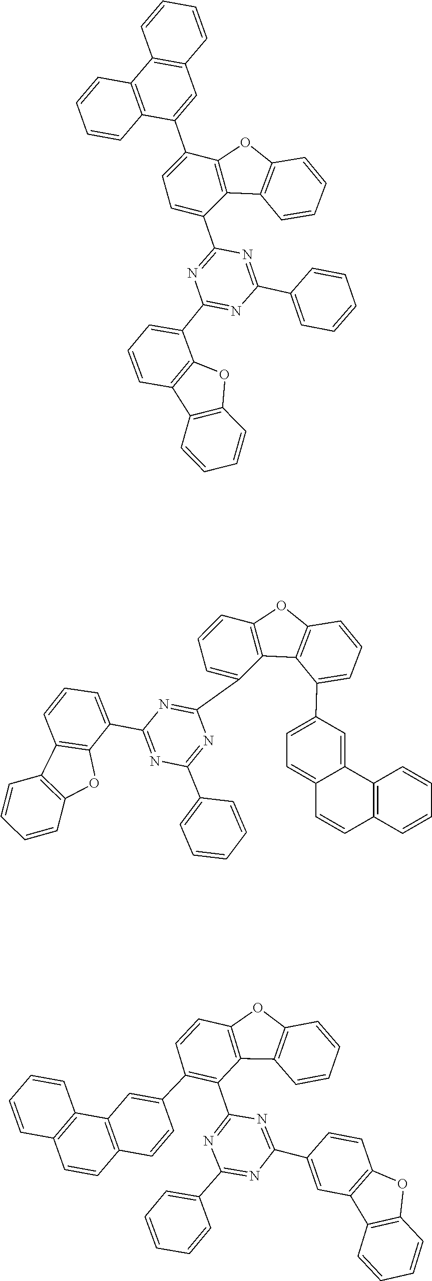 [SMILES: c1ccc(-c2nc(-c3cccc4c3oc3ccccc34)nc(-c3ccc(-c4cc5ccccc5c5ccccc45)c4oc5ccccc5c34)n2)cc1 ; c1ccc(-c2nc(-c3cccc4c3oc3ccccc34)nc(-c3cccc4oc5cccc(-c6ccc7ccc8ccccc8c7c6)c5c34)n2)cc1 ; c1ccc(-c2nc(-c3ccc4oc5ccccc5c4c3)nc(-c3c(-c4ccc5ccc6ccccc6c5c4)ccc4oc5ccccc5c34)n2)cc1]

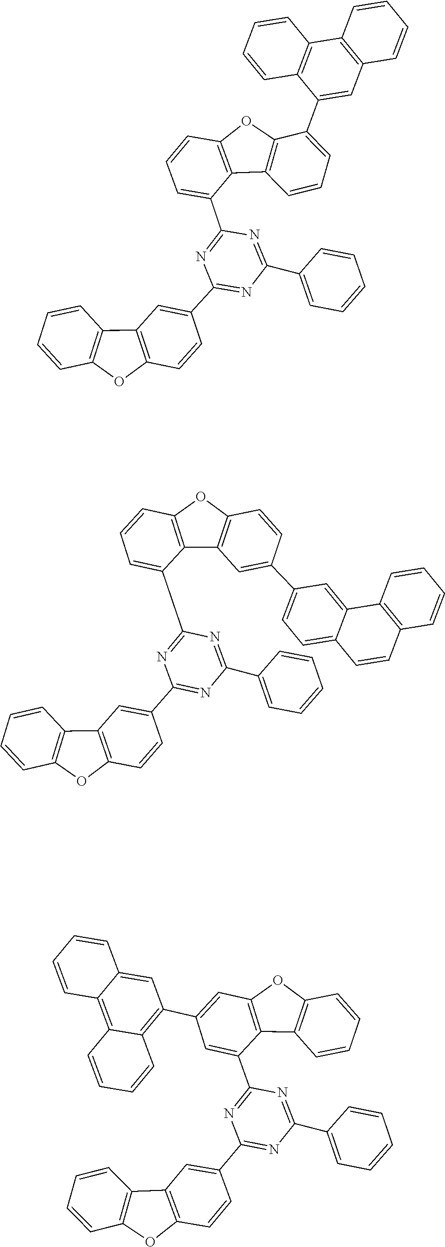 [SMILES: c1ccc(-c2nc(-c3ccc4oc5ccccc5c4c3)nc(-c3cccc4oc5c(-c6cc7ccccc7c7ccccc67)cccc5c34)n2)cc1 ; c1ccc(-c2nc(-c3ccc4oc5ccccc5c4c3)nc(-c3cccc4oc5ccc(-c6ccc7ccc8ccccc8c7c6)cc5c34)n2)cc1 ; c1ccc(-c2nc(-c3ccc4oc5ccccc5c4c3)nc(-c3cc(-c4cc5ccccc5c5ccccc45)cc4oc5ccccc5c34)n2)cc1]

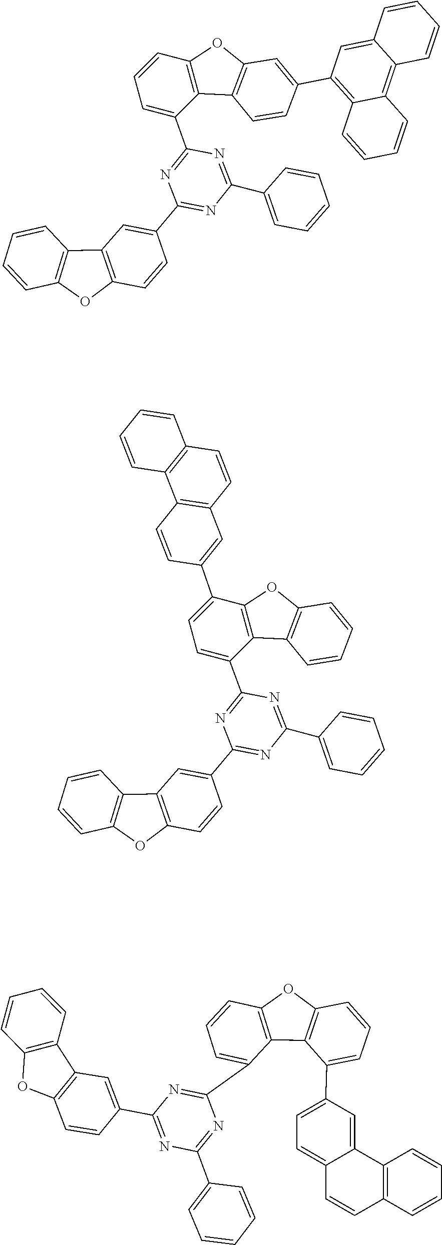 [SMILES: c1ccc(-c2nc(-c3ccc4oc5ccccc5c4c3)nc(-c3cccc4oc5cc(-c6cc7ccccc7c7ccccc67)ccc5c34)n2)cc1 ; c1ccc(-c2nc(-c3ccc4oc5ccccc5c4c3)nc(-c3ccc(-c4ccc5c(ccc6ccccc65)c4)c4oc5ccccc5c34)n2)cc1 ; c1ccc(-c2nc(-c3ccc4oc5ccccc5c4c3)nc(-c3cccc4oc5cccc(-c6ccc7ccc8ccccc8c7c6)c5c34)n2)cc1]

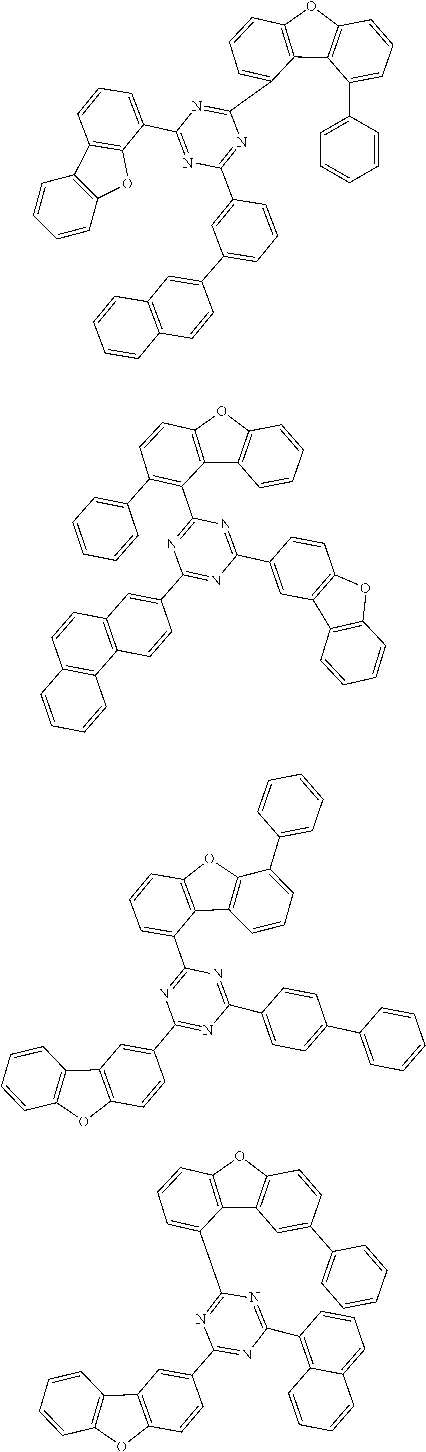 [SMILES: c1ccc(-c2cccc3oc4cccc(-c5nc(-c6cccc(-c7ccc8ccccc8c7)c6)nc(-c6cccc7c6oc6ccccc67)n5)c4c23)cc1 ; c1ccc(-c2ccc3oc4ccccc4c3c2-c2nc(-c3ccc4c(ccc5ccccc54)c3)nc(-c3ccc4oc5ccccc5c4c3)n2)cc1 ; c1ccc(-c2ccc(-c3nc(-c4ccc5oc6ccccc6c5c4)nc(-c4cccc5oc6c(-c7ccccc7)cccc6c45)n3)cc2)cc1 ; c1ccc(-c2ccc3oc4cccc(-c5nc(-c6ccc7oc8ccccc8c7c6)nc(-c6cccc7ccccc67)n5)c4c3c2)cc1]

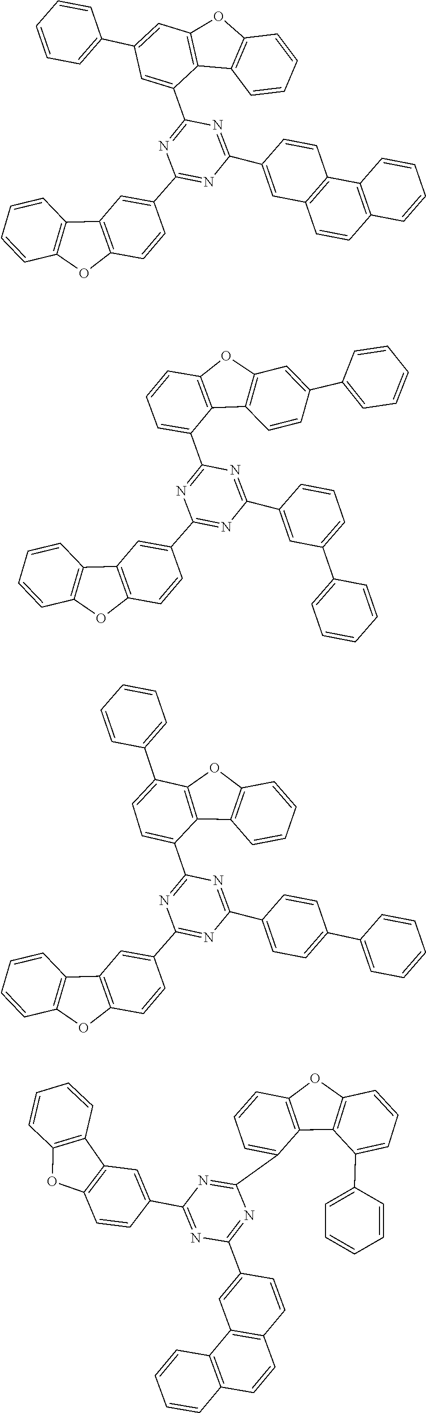 [SMILES: c1ccc(-c2cc(-c3nc(-c4ccc5c(ccc6ccccc65)c4)nc(-c4ccc5oc6ccccc6c5c4)n3)c3c(c2)oc2ccccc23)cc1 ; c1ccc(-c2cccc(-c3nc(-c4ccc5oc6ccccc6c5c4)nc(-c4cccc5oc6cc(-c7ccccc7)ccc6c45)n3)c2)cc1 ; c1ccc(-c2ccc(-c3nc(-c4ccc5oc6ccccc6c5c4)nc(-c4ccc(-c5ccccc5)c5oc6ccccc6c45)n3)cc2)cc1 ; c1ccc(-c2cccc3oc4cccc(-c5nc(-c6ccc7ccc8ccccc8c7c6)nc(-c6ccc7oc8ccccc8c7c6)n5)c4c23)cc1]

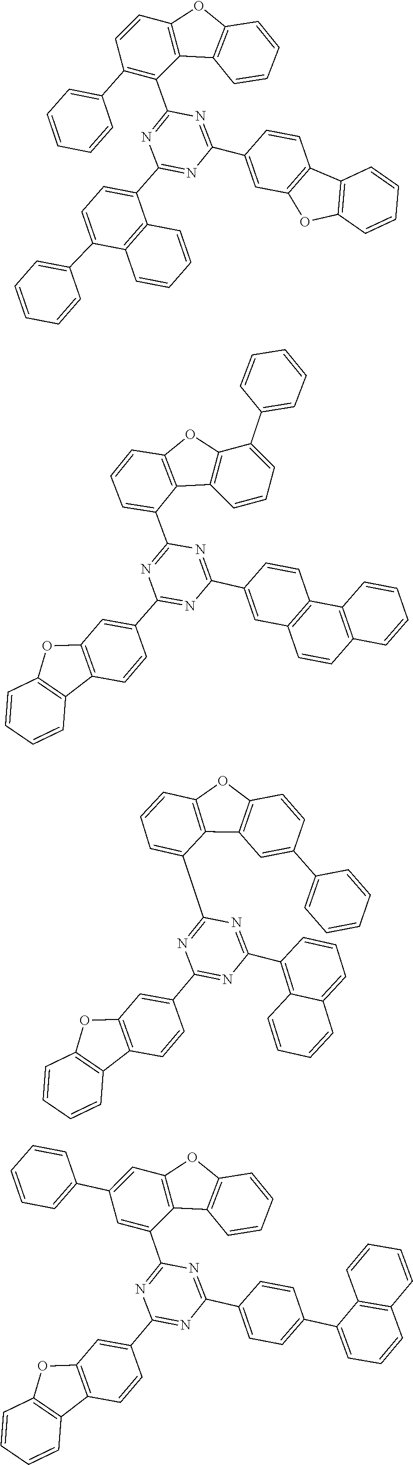

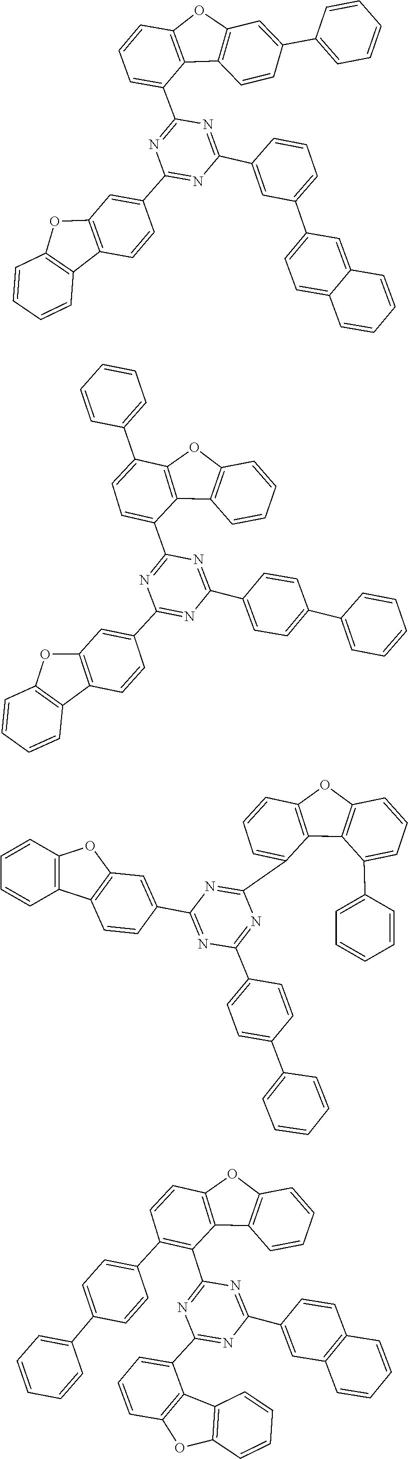 [SMILES: c1ccc(-c2ccc3c(c2)oc2cccc(-c4nc(-c5cccc(-c6ccc7ccccc7c6)c5)nc(-c5ccc6c(c5)oc5ccccc56)n4)c23)cc1 ; c1ccc(-c2ccc(-c3nc(-c4ccc5c(c4)oc4ccccc45)nc(-c4ccc(-c5ccccc5)c5oc6ccccc6c45)n3)cc2)cc1 ; c1ccc(-c2ccc(-c3nc(-c4ccc5c(c4)oc4ccccc45)nc(-c4cccc5oc6cccc(-c7ccccc7)c6c45)n3)cc2)cc1 ; c1ccc(-c2ccc(-c3ccc4oc5ccccc5c4c3-c3nc(-c4ccc5ccccc5c4)nc(-c4cccc5oc6ccccc6c45)n3)cc2)cc1]

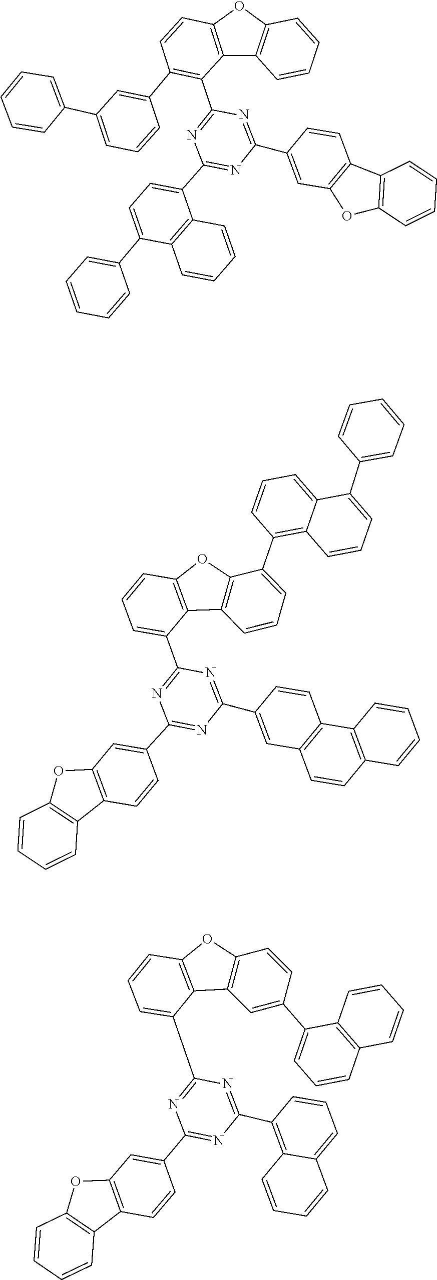 [SMILES: c1ccc(-c2cccc(-c3ccc4oc5ccccc5c4c3-c3nc(-c4ccc5c(c4)oc4ccccc45)nc(-c4ccc(-c5ccccc5)c5ccccc45)n3)c2)cc1 ; c1ccc(-c2cccc3c(-c4cccc5c4oc4cccc(-c6nc(-c7ccc8c(ccc9ccccc98)c7)nc(-c7ccc8c(c7)oc7ccccc78)n6)c45)cccc23)cc1 ; c1ccc2c(-c3ccc4oc5cccc(-c6nc(-c7ccc8c(c7)oc7ccccc78)nc(-c7cccc8ccccc78)n6)c5c4c3)cccc2c1]

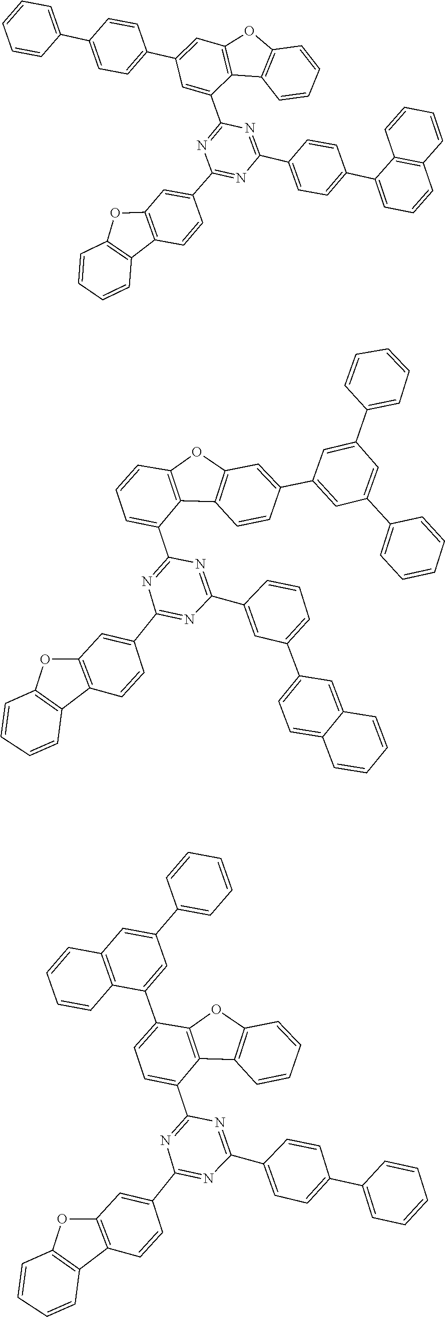 [SMILES: c1ccc(-c2ccc(-c3cc(-c4nc(-c5ccc(-c6cccc7ccccc67)cc5)nc(-c5ccc6c(c5)oc5ccccc56)n4)c4c(c3)oc3ccccc34)cc2)cc1 ; c1ccc(-c2cc(-c3ccccc3)cc(-c3ccc4c(c3)oc3cccc(-c5nc(-c6cccc(-c7ccc8ccccc8c7)c6)nc(-c6ccc7c(c6)oc6ccccc67)n5)c34)c2)cc1 ; c1ccc(-c2ccc(-c3nc(-c4ccc5c(c4)oc4ccccc45)nc(-c4ccc(-c5cc(-c6ccccc6)cc6ccccc56)c5oc6ccccc6c45)n3)cc2)cc1]

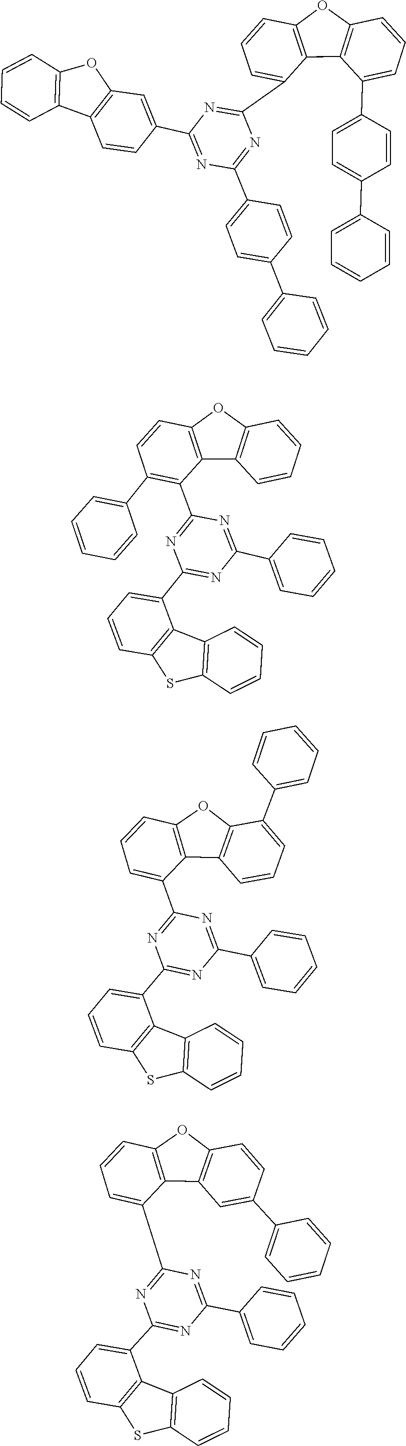 [SMILES: c1ccc(-c2ccc(-c3nc(-c4ccc5c(c4)oc4ccccc45)nc(-c4cccc5oc6cccc(-c7ccc(-c8ccccc8)cc7)c6c45)n3)cc2)cc1 ; c1ccc(-c2nc(-c3c(-c4ccccc4)ccc4oc5ccccc5c34)nc(-c3cccc4sc5ccccc5c34)n2)cc1 ; c1ccc(-c2nc(-c3cccc4oc5c(-c6ccccc6)cccc5c34)nc(-c3cccc4sc5ccccc5c34)n2)cc1 ; c1ccc(-c2ccc3oc4cccc(-c5nc(-c6ccccc6)nc(-c6cccc7sc8ccccc8c67)n5)c4c3c2)cc1]

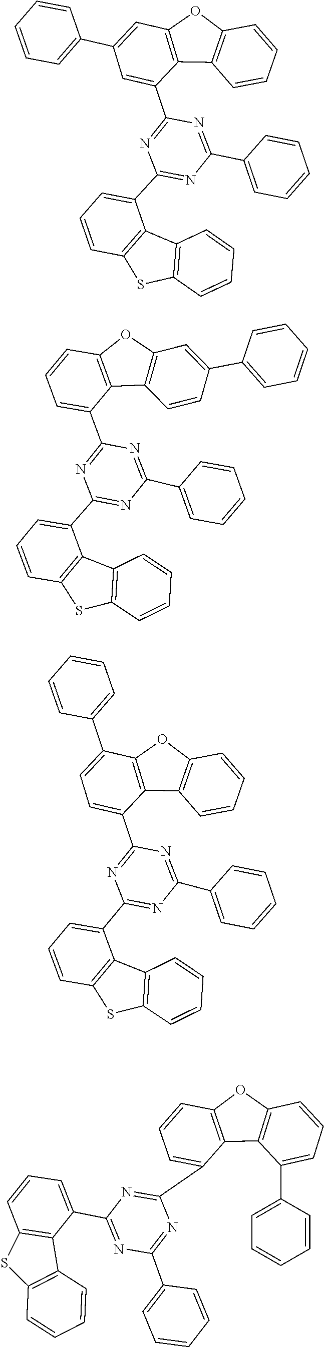

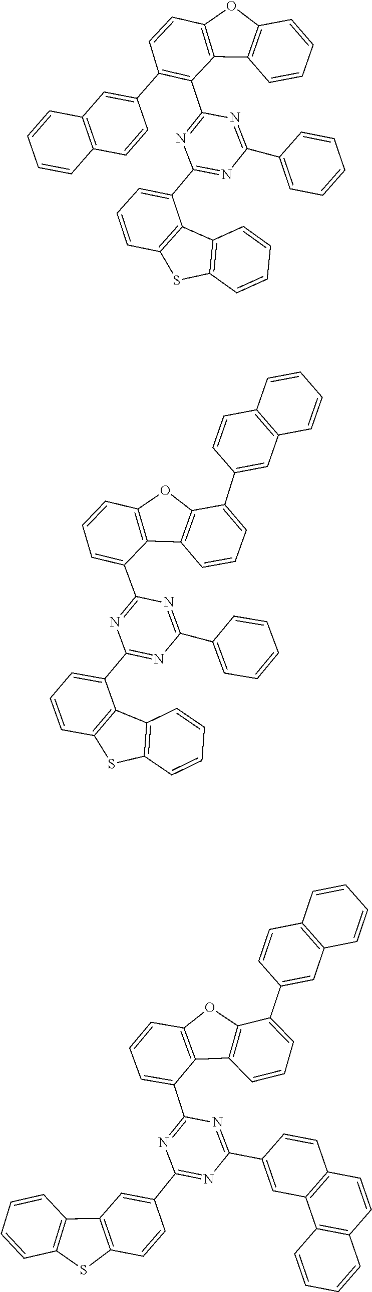 [SMILES: c1ccc(-c2nc(-c3c(-c4ccc5ccccc5c4)ccc4oc5ccccc5c34)nc(-c3cccc4sc5ccccc5c34)n2)cc1 ; c1ccc(-c2nc(-c3cccc4oc5c(-c6ccc7ccccc7c6)cccc5c34)nc(-c3cccc4sc5ccccc5c34)n2)cc1 ; c1ccc2cc(-c3cccc4c3oc3cccc(-c5nc(-c6ccc7ccc8ccccc8c7c6)nc(-c6ccc7sc8ccccc8c7c6)n5)c34)ccc2c1]

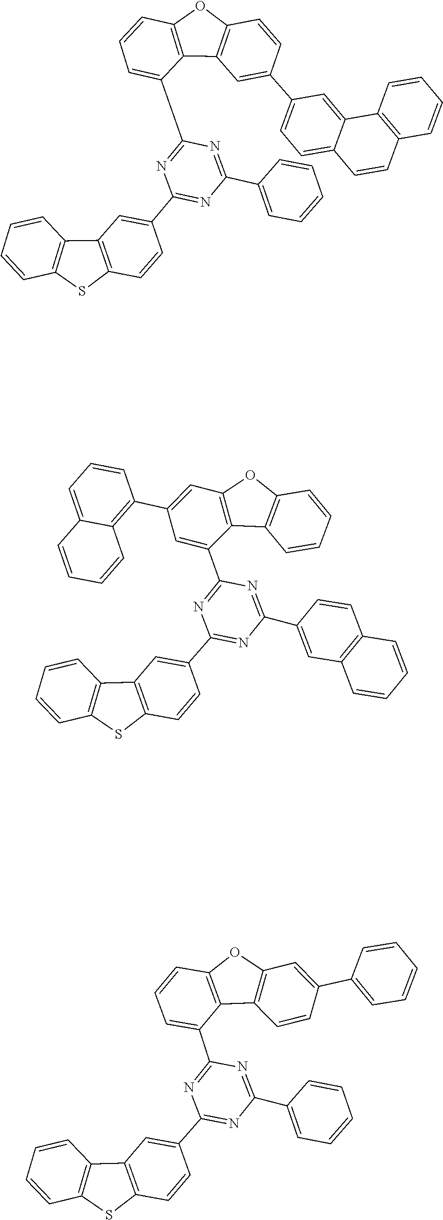 [SMILES: c1ccc(-c2nc(-c3ccc4sc5ccccc5c4c3)nc(-c3cccc4oc5ccc(-c6ccc7ccc8ccccc8c7c6)cc5c34)n2)cc1 ; c1ccc2cc(-c3nc(-c4ccc5sc6ccccc6c5c4)nc(-c4cc(-c5cccc6ccccc56)cc5oc6ccccc6c45)n3)ccc2c1 ; c1ccc(-c2ccc3c(c2)oc2cccc(-c4nc(-c5ccccc5)nc(-c5ccc6sc7ccccc7c6c5)n4)c23)cc1]

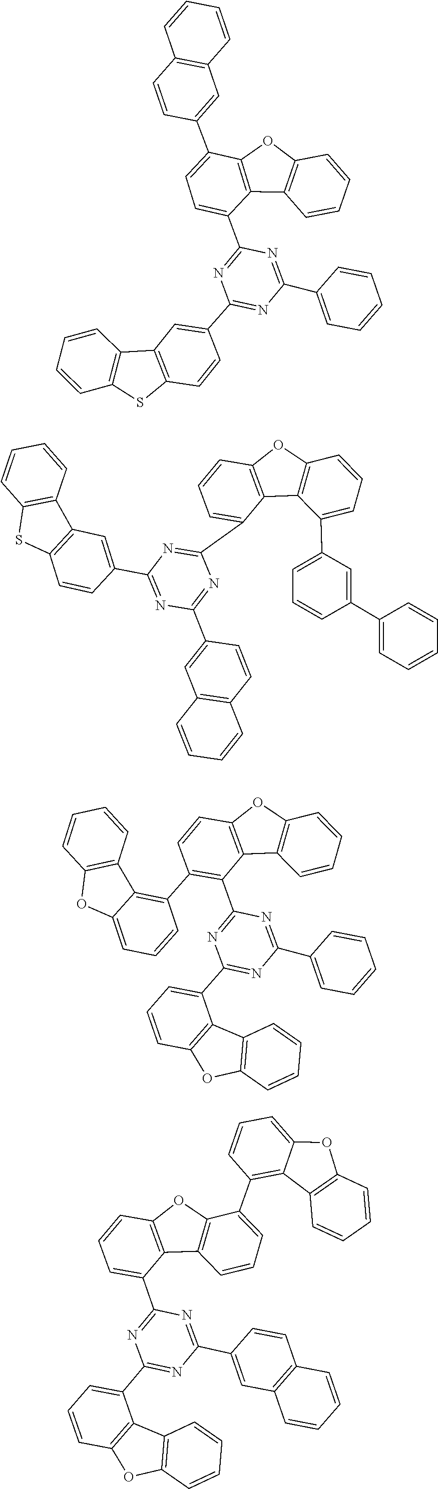

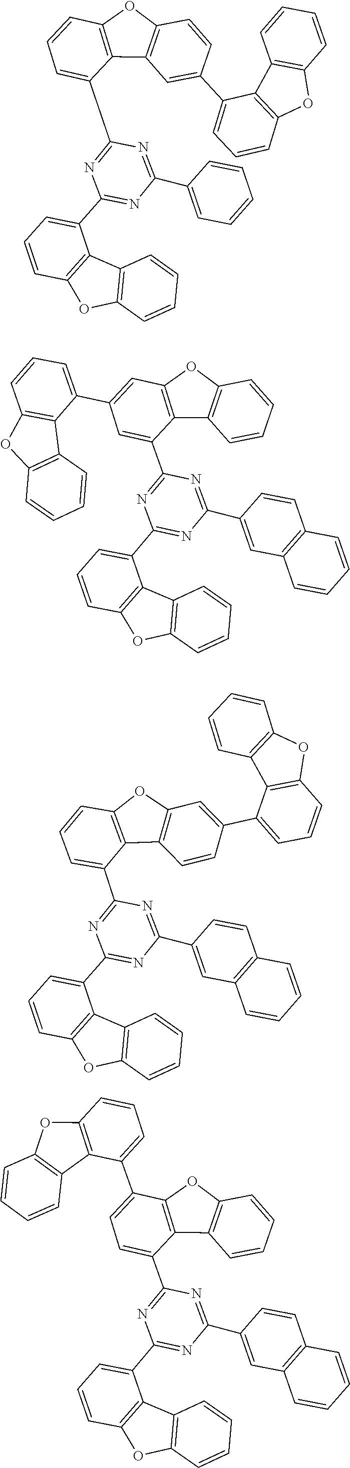

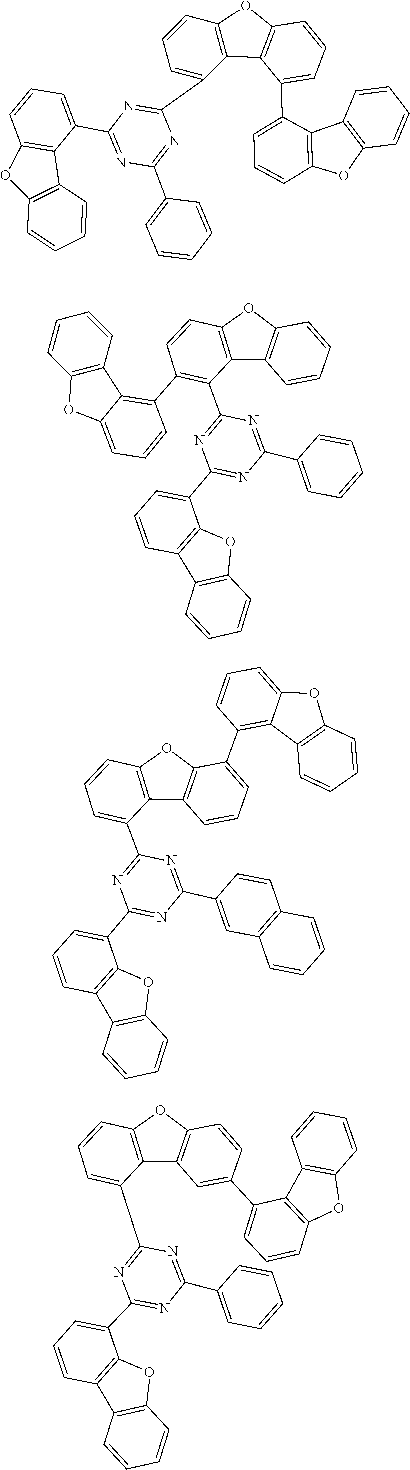

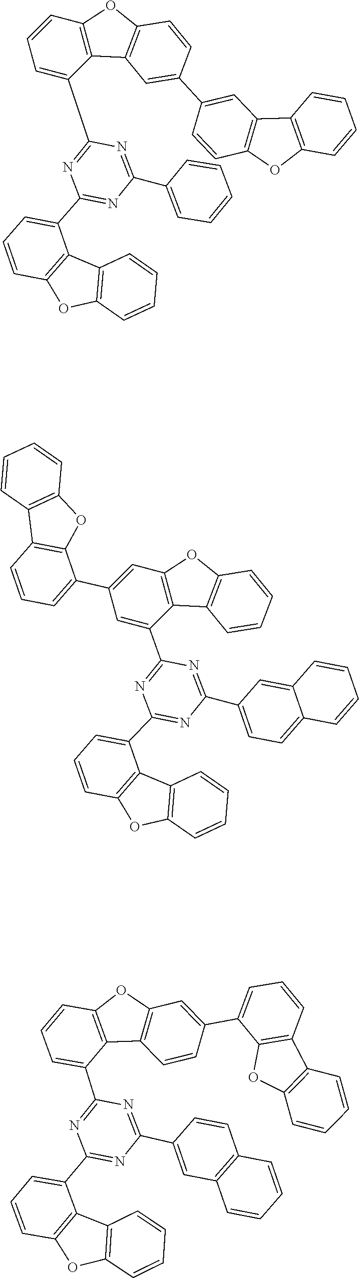 [SMILES: c1ccc(-c2nc(-c3cccc4oc5ccccc5c34)nc(-c3cccc4oc5ccc(-c6ccc7oc8ccccc8c7c6)cc5c34)n2)cc1 ; c1ccc2cc(-c3nc(-c4cccc5oc6ccccc6c45)nc(-c4cc(-c5cccc6c5oc5ccccc56)cc5oc6ccccc6c45)n3)ccc2c1 ; c1ccc2cc(-c3nc(-c4cccc5oc6ccccc6c45)nc(-c4cccc5oc6cc(-c7cccc8c7oc7ccccc78)ccc6c45)n3)ccc2c1]

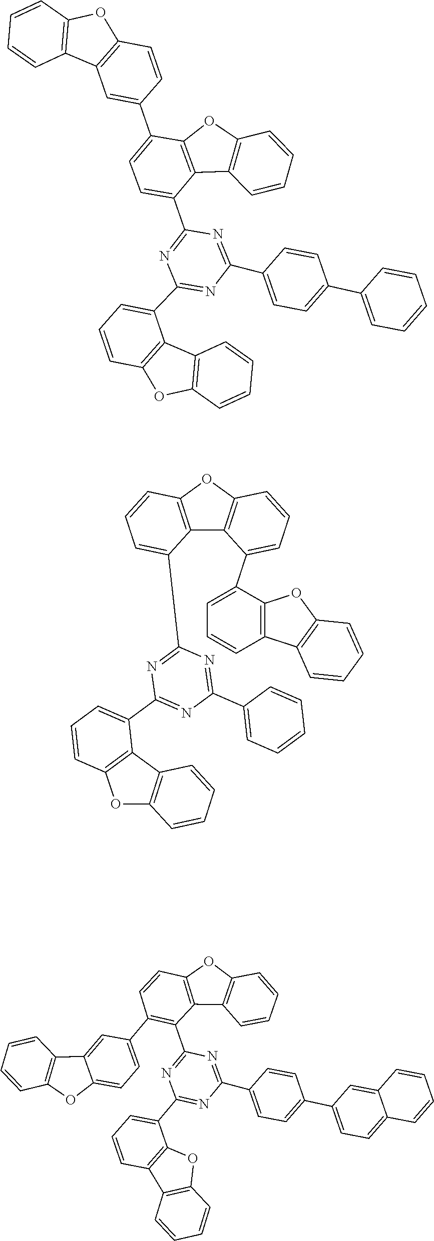 [SMILES: c1ccc(-c2ccc(-c3nc(-c4cccc5oc6ccccc6c45)nc(-c4ccc(-c5ccc6oc7ccccc7c6c5)c5oc6ccccc6c45)n3)cc2)cc1 ; c1ccc(-c2nc(-c3cccc4oc5ccccc5c34)nc(-c3cccc4oc5cccc(-c6cccc7c6oc6ccccc67)c5c34)n2)cc1 ; c1ccc2cc(-c3ccc(-c4nc(-c5cccc6c5oc5ccccc56)nc(-c5c(-c6ccc7oc8ccccc8c7c6)ccc6oc7ccccc7c56)n4)cc3)ccc2c1]

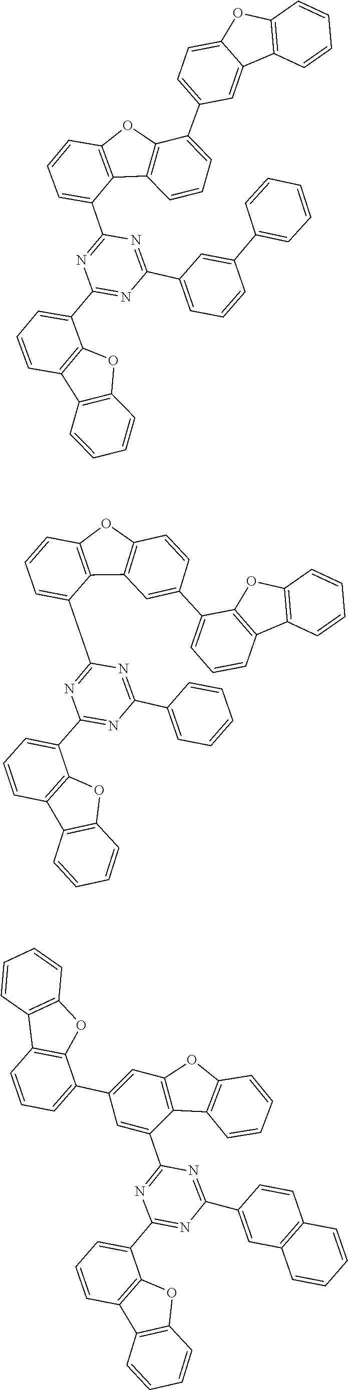 [SMILES: c1ccc(-c2cccc(-c3nc(-c4cccc5c4oc4ccccc45)nc(-c4cccc5oc6c(-c7ccc8oc9ccccc9c8c7)cccc6c45)n3)c2)cc1 ; c1ccc(-c2nc(-c3cccc4c3oc3ccccc34)nc(-c3cccc4oc5ccc(-c6cccc7c6oc6ccccc67)cc5c34)n2)cc1 ; c1ccc2cc(-c3nc(-c4cccc5c4oc4ccccc45)nc(-c4cc(-c5cccc6c5oc5ccccc56)cc5oc6ccccc6c45)n3)ccc2c1]

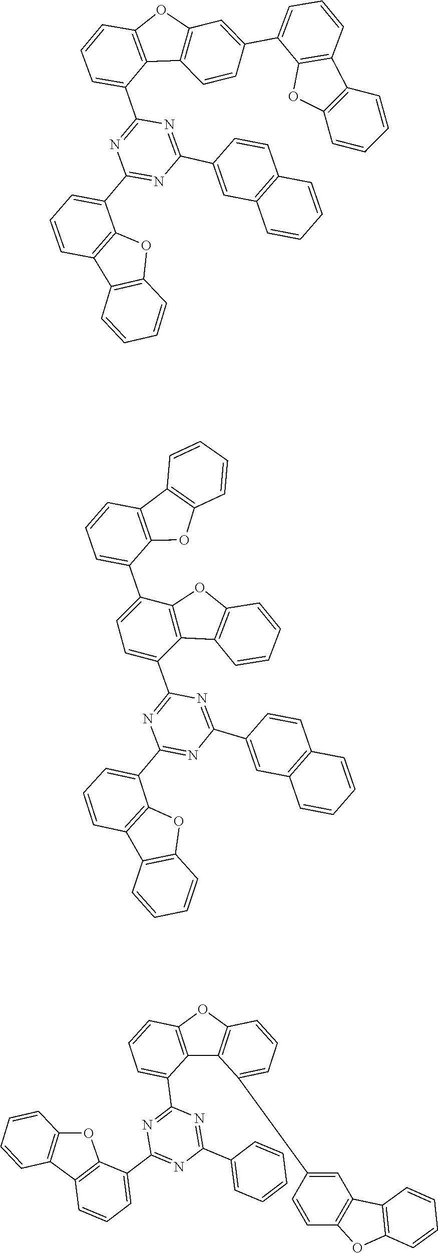 [SMILES: c1ccc2cc(-c3nc(-c4cccc5c4oc4ccccc45)nc(-c4cccc5oc6cc(-c7cccc8c7oc7ccccc78)ccc6c45)n3)ccc2c1 ; c1ccc2cc(-c3nc(-c4cccc5c4oc4ccccc45)nc(-c4ccc(-c5cccc6c5oc5ccccc56)c5oc6ccccc6c45)n3)ccc2c1 ; c1ccc(-c2nc(-c3cccc4c3oc3ccccc34)nc(-c3cccc4oc5cccc(-c6ccc7oc8ccccc8c7c6)c5c34)n2)cc1]

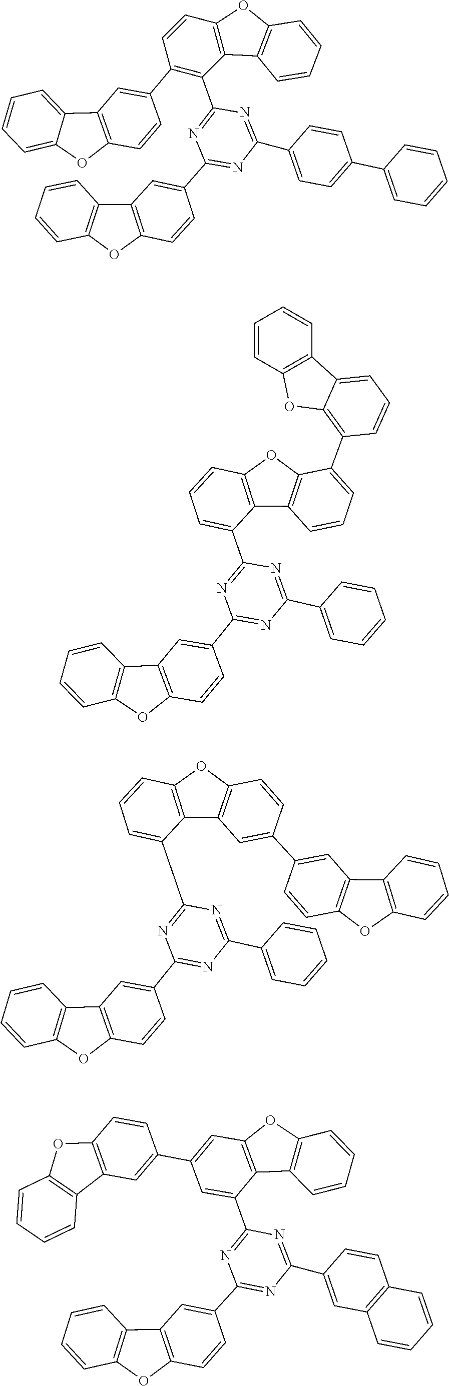 [SMILES: c1ccc(-c2ccc(-c3nc(-c4ccc5oc6ccccc6c5c4)nc(-c4c(-c5ccc6oc7ccccc7c6c5)ccc5oc6ccccc6c45)n3)cc2)cc1 ; c1ccc(-c2nc(-c3ccc4oc5ccccc5c4c3)nc(-c3cccc4oc5c(-c6cccc7c6oc6ccccc67)cccc5c34)n2)cc1 ; c1ccc(-c2nc(-c3ccc4oc5ccccc5c4c3)nc(-c3cccc4oc5ccc(-c6ccc7oc8ccccc8c7c6)cc5c34)n2)cc1 ; c1ccc2cc(-c3nc(-c4ccc5oc6ccccc6c5c4)nc(-c4cc(-c5ccc6oc7ccccc7c6c5)cc5oc6ccccc6c45)n3)ccc2c1]

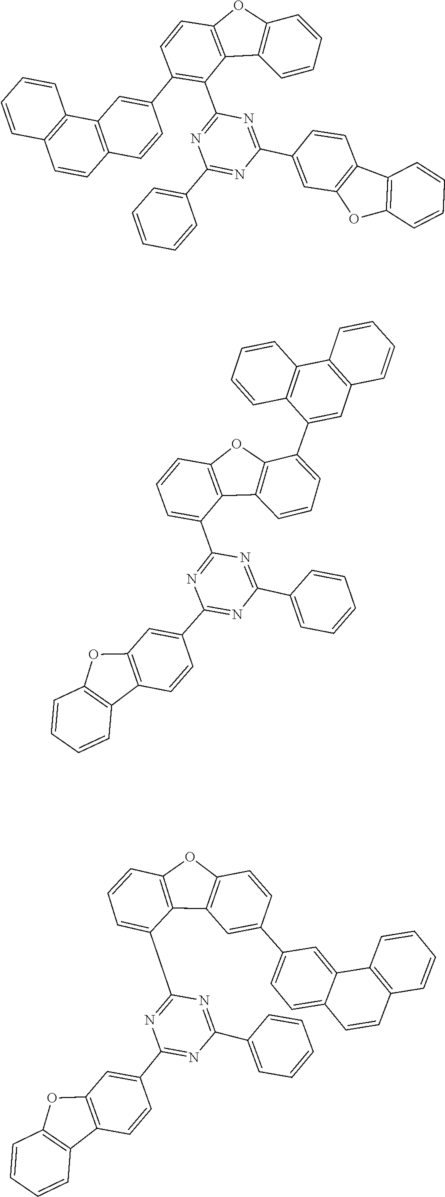 [SMILES: c1ccc(-c2nc(-c3ccc4c(c3)oc3ccccc34)nc(-c3c(-c4ccc5ccc6ccccc6c5c4)ccc4oc5ccccc5c34)n2)cc1 ; c1ccc(-c2nc(-c3ccc4c(c3)oc3ccccc34)nc(-c3cccc4oc5c(-c6cc7ccccc7c7ccccc67)cccc5c34)n2)cc1 ; c1ccc(-c2nc(-c3ccc4c(c3)oc3ccccc34)nc(-c3cccc4oc5ccc(-c6ccc7ccc8ccccc8c7c6)cc5c34)n2)cc1]

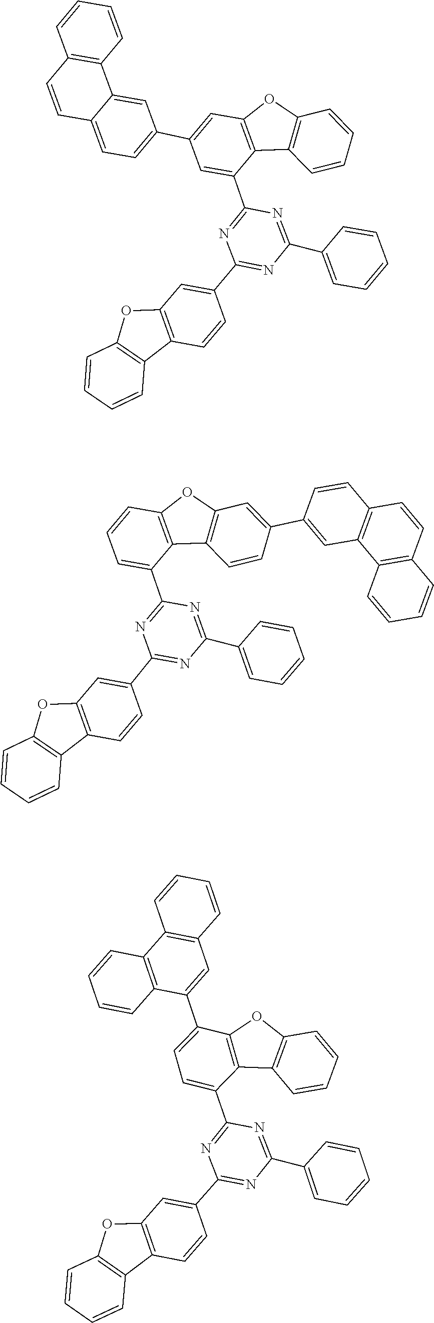 [SMILES: c1ccc(-c2nc(-c3ccc4c(c3)oc3ccccc34)nc(-c3cc(-c4ccc5ccc6ccccc6c5c4)cc4oc5ccccc5c34)n2)cc1 ; c1ccc(-c2nc(-c3ccc4c(c3)oc3ccccc34)nc(-c3cccc4oc5cc(-c6ccc7ccc8ccccc8c7c6)ccc5c34)n2)cc1 ; c1ccc(-c2nc(-c3ccc4c(c3)oc3ccccc34)nc(-c3ccc(-c4cc5ccccc5c5ccccc45)c4oc5ccccc5c34)n2)cc1]

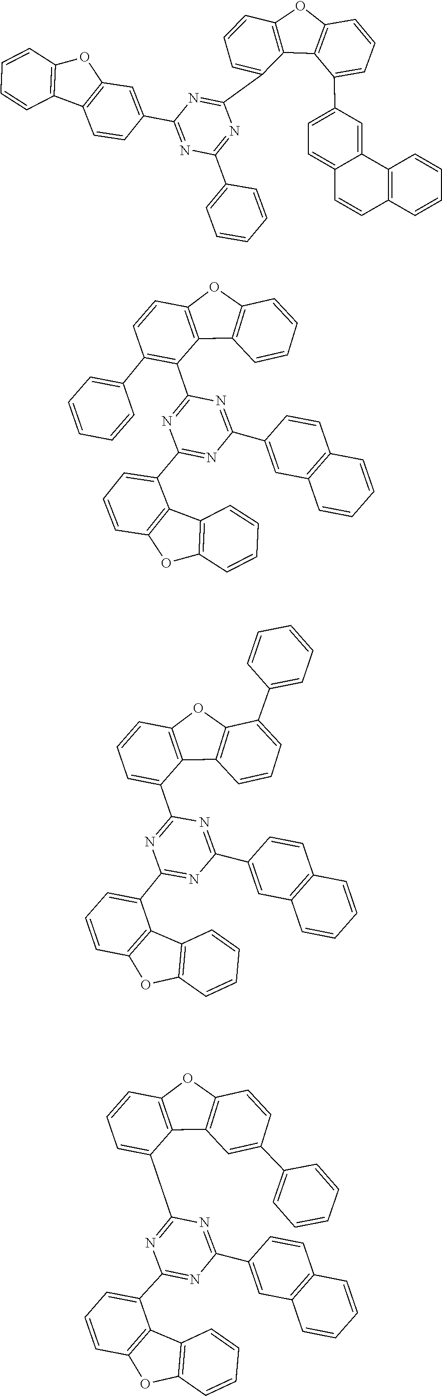 [SMILES: c1ccc(-c2nc(-c3ccc4c(c3)oc3ccccc34)nc(-c3cccc4oc5cccc(-c6ccc7ccc8ccccc8c7c6)c5c34)n2)cc1 ; c1ccc(-c2ccc3oc4ccccc4c3c2-c2nc(-c3ccc4ccccc4c3)nc(-c3cccc4oc5ccccc5c34)n2)cc1 ; c1ccc(-c2cccc3c2oc2cccc(-c4nc(-c5ccc6ccccc6c5)nc(-c5cccc6oc7ccccc7c56)n4)c23)cc1 ; c1ccc(-c2ccc3oc4cccc(-c5nc(-c6ccc7ccccc7c6)nc(-c6cccc7oc8ccccc8c67)n5)c4c3c2)cc1]

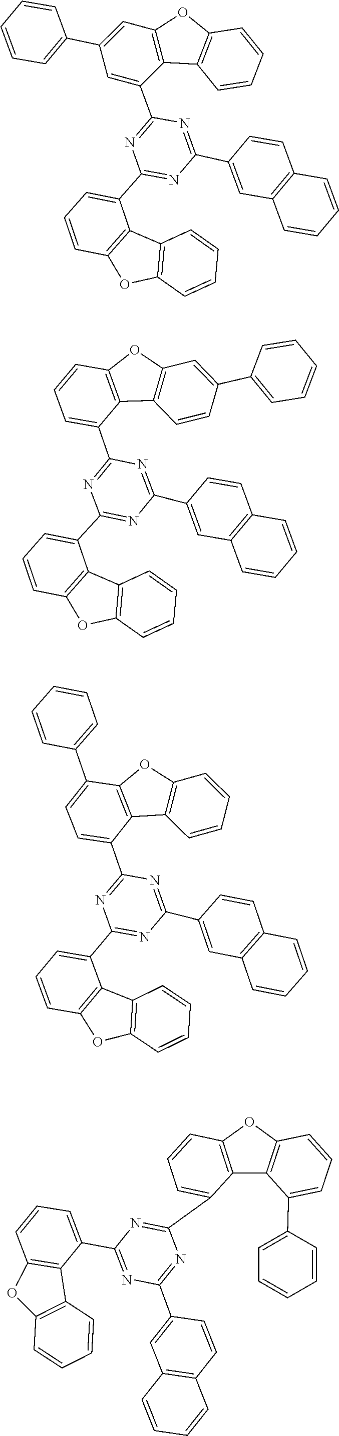 [SMILES: c1ccc(-c2cc(-c3nc(-c4ccc5ccccc5c4)nc(-c4cccc5oc6ccccc6c45)n3)c3c(c2)oc2ccccc23)cc1 ; c1ccc(-c2ccc3c(c2)oc2cccc(-c4nc(-c5ccc6ccccc6c5)nc(-c5cccc6oc7ccccc7c56)n4)c23)cc1 ; c1ccc(-c2ccc(-c3nc(-c4ccc5ccccc5c4)nc(-c4cccc5oc6ccccc6c45)n3)c3c2oc2ccccc23)cc1 ; c1ccc(-c2cccc3oc4cccc(-c5nc(-c6ccc7ccccc7c6)nc(-c6cccc7oc8ccccc8c67)n5)c4c23)cc1]

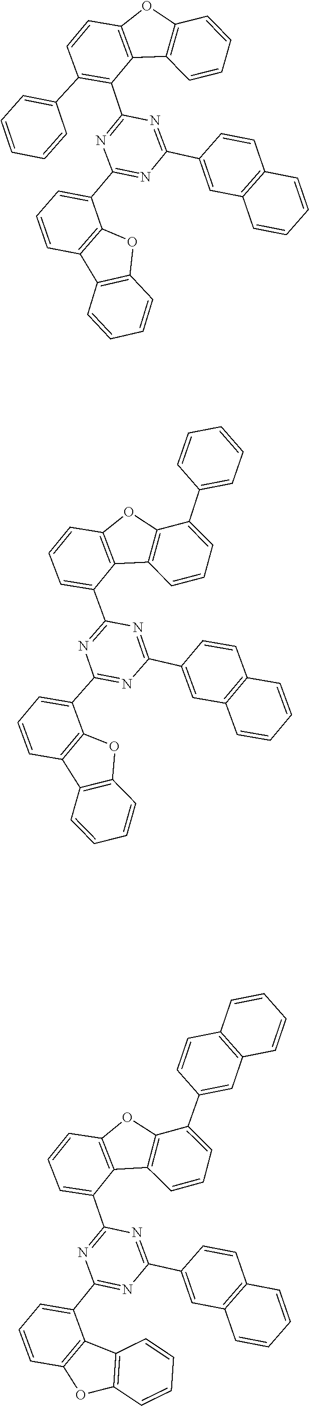 [SMILES: c1ccc(-c2ccc3oc4ccccc4c3c2-c2nc(-c3ccc4ccccc4c3)nc(-c3cccc4c3oc3ccccc34)n2)cc1 ; c1ccc(-c2cccc3c2oc2cccc(-c4nc(-c5ccc6ccccc6c5)nc(-c5cccc6c5oc5ccccc56)n4)c23)cc1 ; c1ccc2cc(-c3nc(-c4cccc5oc6ccccc6c45)nc(-c4cccc5oc6c(-c7ccc8ccccc8c7)cccc6c45)n3)ccc2c1]

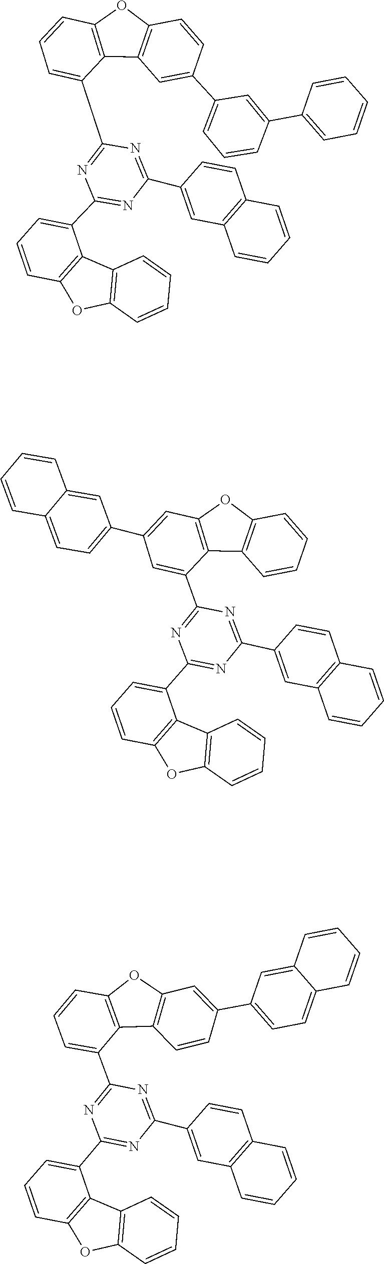 [SMILES: c1ccc(-c2cccc(-c3ccc4oc5cccc(-c6nc(-c7ccc8ccccc8c7)nc(-c7cccc8oc9ccccc9c78)n6)c5c4c3)c2)cc1 ; c1ccc2cc(-c3cc(-c4nc(-c5ccc6ccccc6c5)nc(-c5cccc6oc7ccccc7c56)n4)c4c(c3)oc3ccccc34)ccc2c1 ; c1ccc2cc(-c3ccc4c(c3)oc3cccc(-c5nc(-c6ccc7ccccc7c6)nc(-c6cccc7oc8ccccc8c67)n5)c34)ccc2c1]

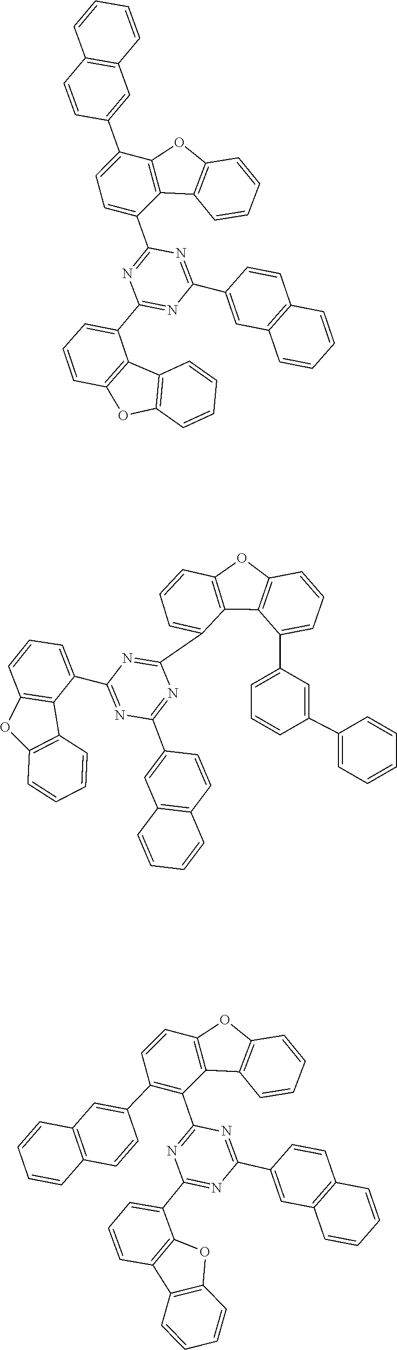 [SMILES: c1ccc2cc(-c3nc(-c4cccc5oc6ccccc6c45)nc(-c4ccc(-c5ccc6ccccc6c5)c5oc6ccccc6c45)n3)ccc2c1 ; c1ccc(-c2cccc(-c3cccc4oc5cccc(-c6nc(-c7ccc8ccccc8c7)nc(-c7cccc8oc9ccccc9c78)n6)c5c34)c2)cc1 ; c1ccc2cc(-c3nc(-c4cccc5c4oc4ccccc45)nc(-c4c(-c5ccc6ccccc6c5)ccc5oc6ccccc6c45)n3)ccc2c1]

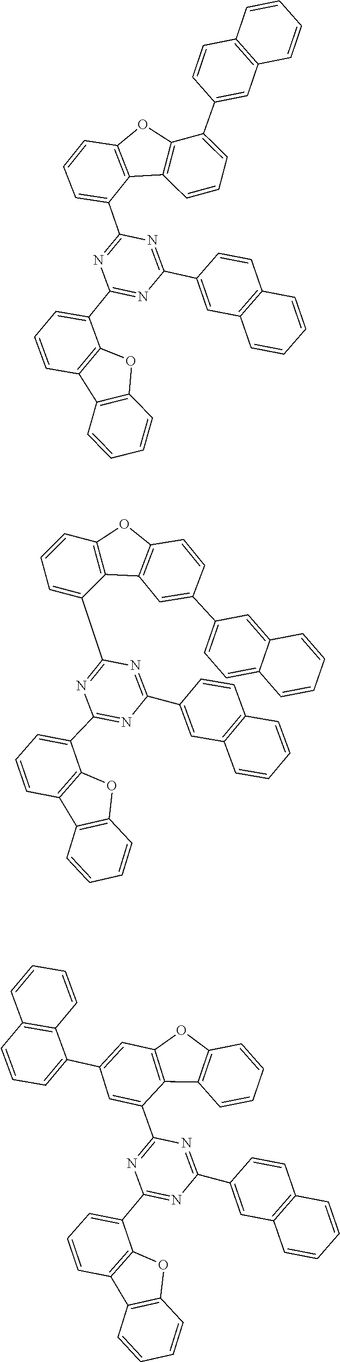 [SMILES: c1ccc2cc(-c3nc(-c4cccc5c4oc4ccccc45)nc(-c4cccc5oc6c(-c7ccc8ccccc8c7)cccc6c45)n3)ccc2c1 ; c1ccc2cc(-c3ccc4oc5cccc(-c6nc(-c7ccc8ccccc8c7)nc(-c7cccc8c7oc7ccccc78)n6)c5c4c3)ccc2c1 ; c1ccc2cc(-c3nc(-c4cccc5c4oc4ccccc45)nc(-c4cc(-c5cccc6ccccc56)cc5oc6ccccc6c45)n3)ccc2c1]

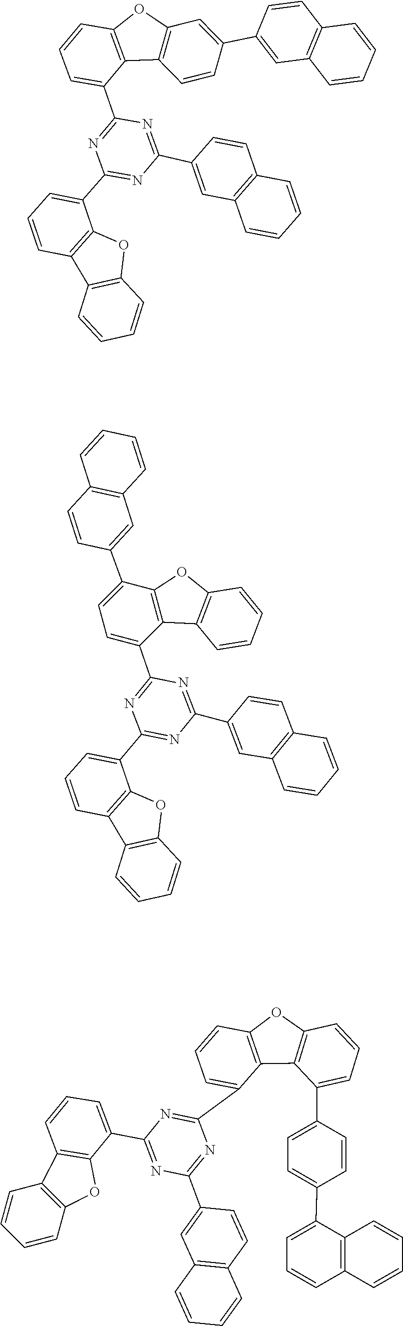 [SMILES: c1ccc2cc(-c3ccc4c(c3)oc3cccc(-c5nc(-c6ccc7ccccc7c6)nc(-c6cccc7c6oc6ccccc67)n5)c34)ccc2c1 ; c1ccc2cc(-c3nc(-c4cccc5c4oc4ccccc45)nc(-c4ccc(-c5ccc6ccccc6c5)c5oc6ccccc6c45)n3)ccc2c1 ; c1ccc2cc(-c3nc(-c4cccc5c4oc4ccccc45)nc(-c4cccc5oc6cccc(-c7ccc(-c8cccc9ccccc89)cc7)c6c45)n3)ccc2c1]

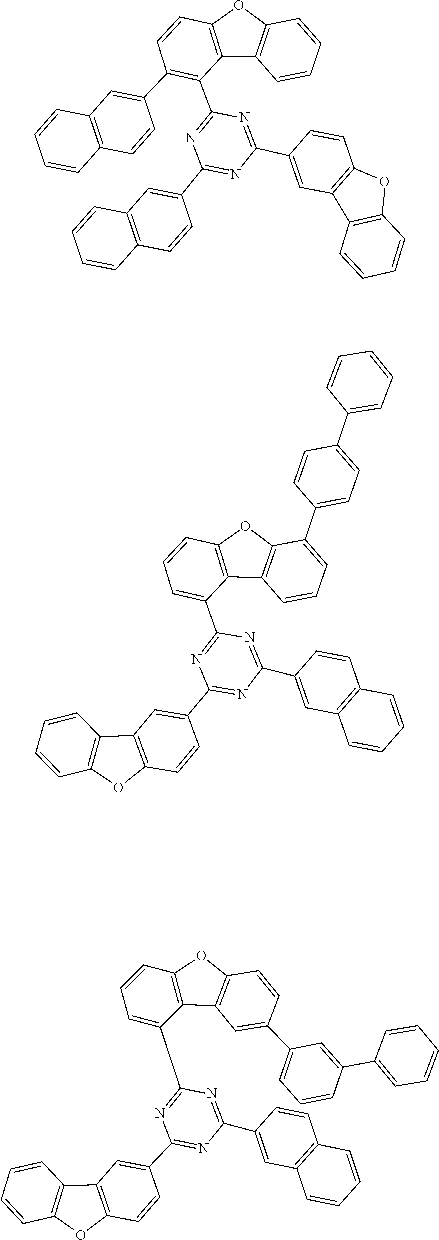 [SMILES: c1ccc2cc(-c3nc(-c4ccc5oc6ccccc6c5c4)nc(-c4c(-c5ccc6ccccc6c5)ccc5oc6ccccc6c45)n3)ccc2c1 ; c1ccc(-c2ccc(-c3cccc4c3oc3cccc(-c5nc(-c6ccc7ccccc7c6)nc(-c6ccc7oc8ccccc8c7c6)n5)c34)cc2)cc1 ; c1ccc(-c2cccc(-c3ccc4oc5cccc(-c6nc(-c7ccc8ccccc8c7)nc(-c7ccc8oc9ccccc9c8c7)n6)c5c4c3)c2)cc1]

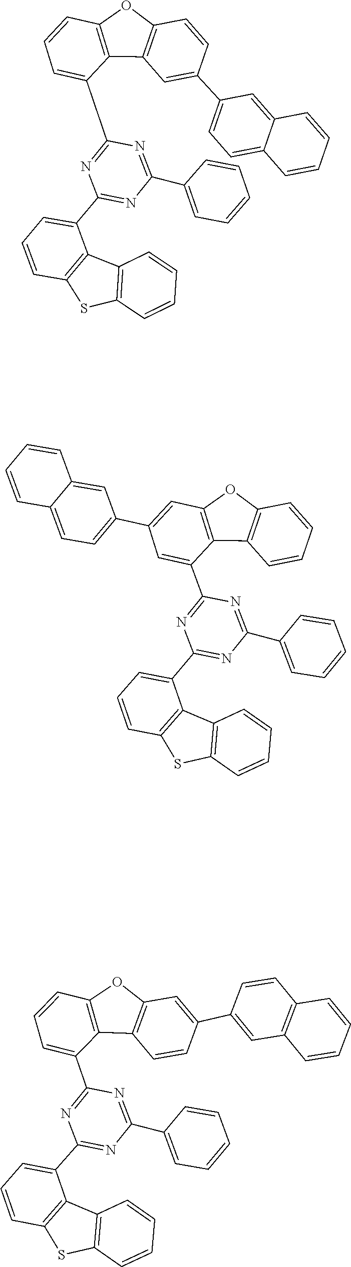 [SMILES: c1ccc(-c2nc(-c3cccc4oc5ccc(-c6ccc7ccccc7c6)cc5c34)nc(-c3cccc4sc5ccccc5c34)n2)cc1 ; c1ccc(-c2nc(-c3cc(-c4ccc5ccccc5c4)cc4oc5ccccc5c34)nc(-c3cccc4sc5ccccc5c34)n2)cc1 ; c1ccc(-c2nc(-c3cccc4oc5cc(-c6ccc7ccccc7c6)ccc5c34)nc(-c3cccc4sc5ccccc5c34)n2)cc1]

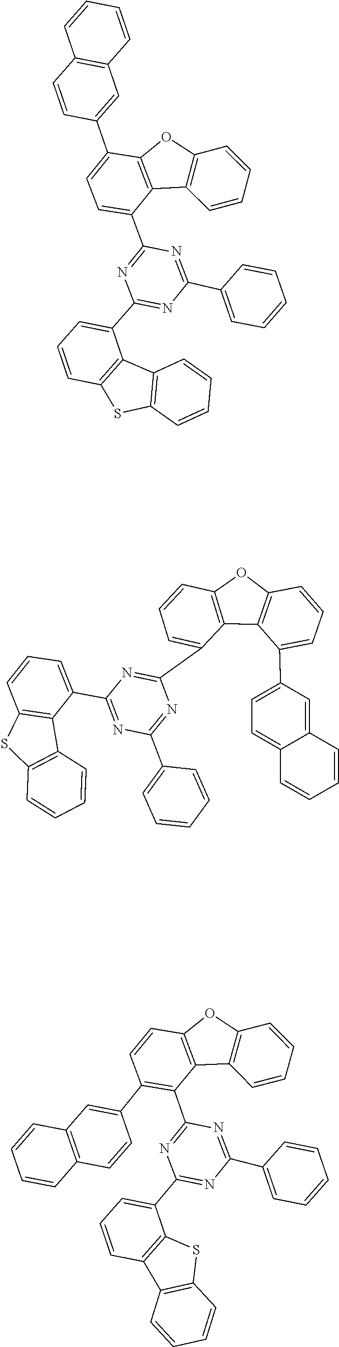 [SMILES: c1ccc(-c2nc(-c3cccc4sc5ccccc5c34)nc(-c3ccc(-c4ccc5ccccc5c4)c4oc5ccccc5c34)n2)cc1 ; c1ccc(-c2nc(-c3cccc4sc5ccccc5c34)nc(-c3cccc4oc5cccc(-c6ccc7ccccc7c6)c5c34)n2)cc1 ; c1ccc(-c2nc(-c3cccc4c3sc3ccccc34)nc(-c3c(-c4ccc5ccccc5c4)ccc4oc5ccccc5c34)n2)cc1]

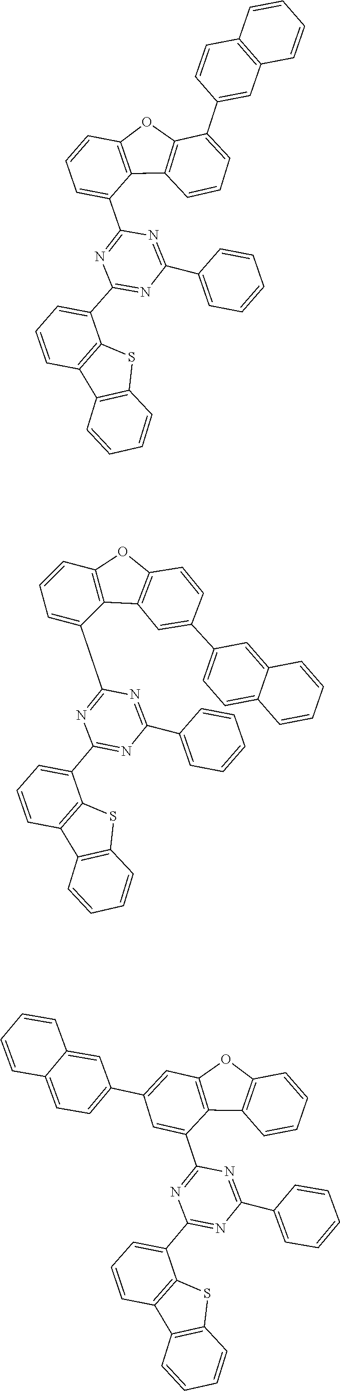 [SMILES: c1ccc(-c2nc(-c3cccc4c3sc3ccccc34)nc(-c3cccc4oc5c(-c6ccc7ccccc7c6)cccc5c34)n2)cc1 ; c1ccc(-c2nc(-c3cccc4c3sc3ccccc34)nc(-c3cccc4oc5ccc(-c6ccc7ccccc7c6)cc5c34)n2)cc1 ; c1ccc(-c2nc(-c3cccc4c3sc3ccccc34)nc(-c3cc(-c4ccc5ccccc5c4)cc4oc5ccccc5c34)n2)cc1]

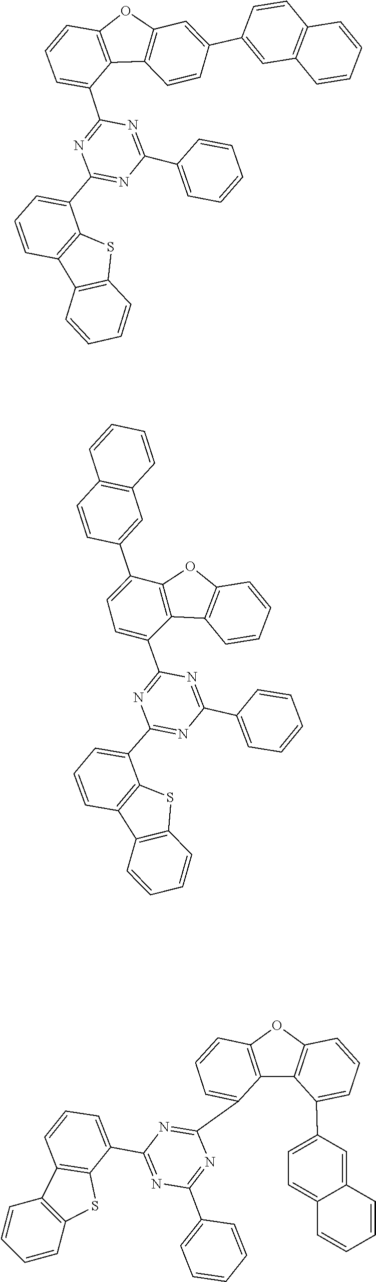 [SMILES: c1ccc(-c2nc(-c3cccc4c3sc3ccccc34)nc(-c3cccc4oc5cc(-c6ccc7ccccc7c6)ccc5c34)n2)cc1 ; c1ccc(-c2nc(-c3cccc4c3sc3ccccc34)nc(-c3ccc(-c4ccc5ccccc5c4)c4oc5ccccc5c34)n2)cc1 ; c1ccc(-c2nc(-c3cccc4c3sc3ccccc34)nc(-c3cccc4oc5cccc(-c6ccc7ccccc7c6)c5c34)n2)cc1]

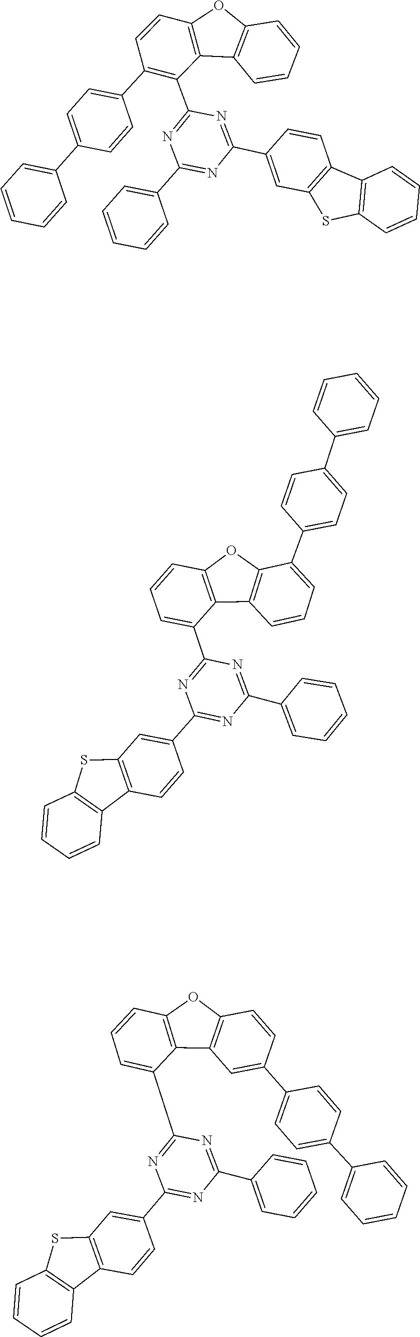 [SMILES: c1ccc(-c2ccc(-c3ccc4oc5ccccc5c4c3-c3nc(-c4ccccc4)nc(-c4ccc5c(c4)sc4ccccc45)n3)cc2)cc1 ; c1ccc(-c2ccc(-c3cccc4c3oc3cccc(-c5nc(-c6ccccc6)nc(-c6ccc7c(c6)sc6ccccc67)n5)c34)cc2)cc1 ; c1ccc(-c2ccc(-c3ccc4oc5cccc(-c6nc(-c7ccccc7)nc(-c7ccc8c(c7)sc7ccccc78)n6)c5c4c3)cc2)cc1]

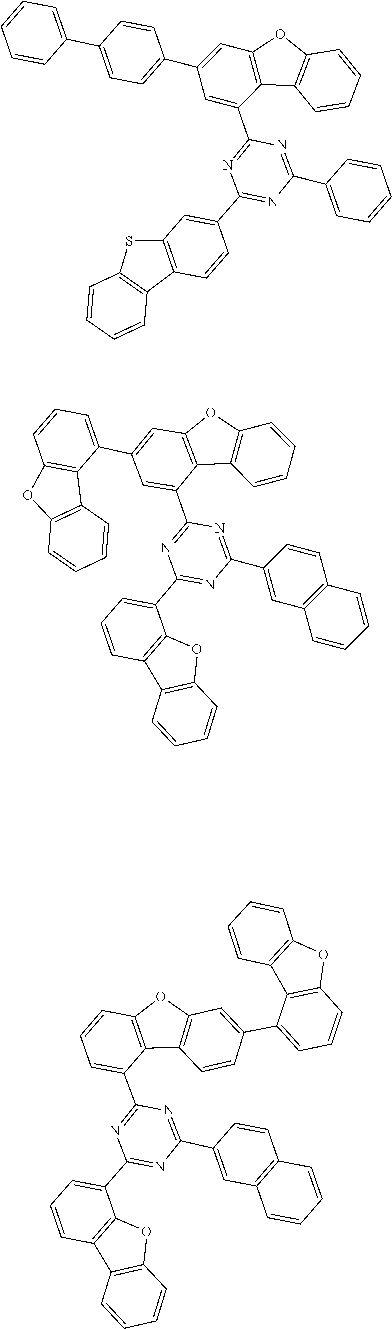 [SMILES: c1ccc(-c2ccc(-c3cc(-c4nc(-c5ccccc5)nc(-c5ccc6c(c5)sc5ccccc56)n4)c4c(c3)oc3ccccc34)cc2)cc1 ; c1ccc2cc(-c3nc(-c4cccc5c4oc4ccccc45)nc(-c4cc(-c5cccc6oc7ccccc7c56)cc5oc6ccccc6c45)n3)ccc2c1 ; c1ccc2cc(-c3nc(-c4cccc5c4oc4ccccc45)nc(-c4cccc5oc6cc(-c7cccc8oc9ccccc9c78)ccc6c45)n3)ccc2c1]

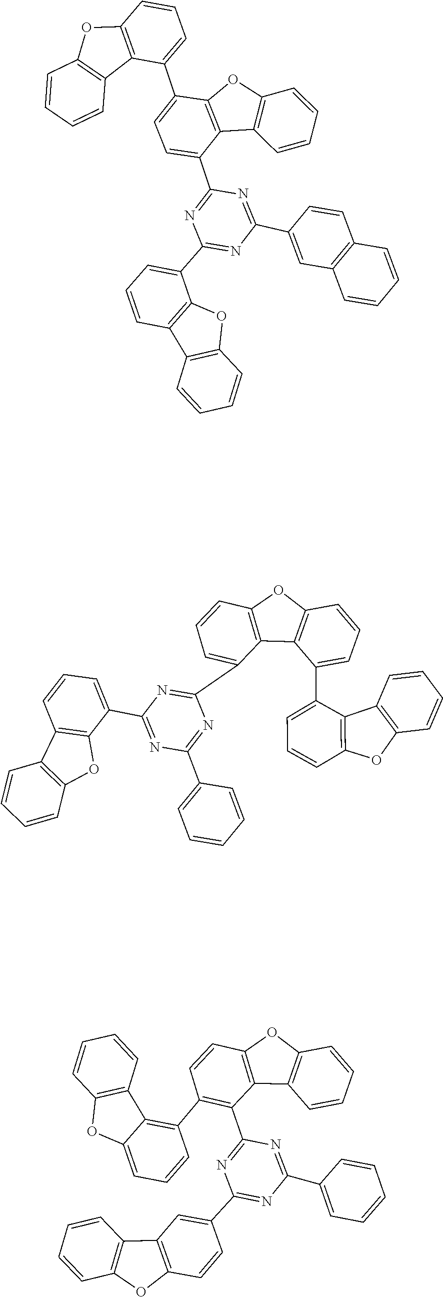 [SMILES: c1ccc2cc(-c3nc(-c4cccc5c4oc4ccccc45)nc(-c4ccc(-c5cccc6oc7ccccc7c56)c5oc6ccccc6c45)n3)ccc2c1 ; c1ccc(-c2nc(-c3cccc4c3oc3ccccc34)nc(-c3cccc4oc5cccc(-c6cccc7oc8ccccc8c67)c5c34)n2)cc1 ; c1ccc(-c2nc(-c3ccc4oc5ccccc5c4c3)nc(-c3c(-c4cccc5oc6ccccc6c45)ccc4oc5ccccc5c34)n2)cc1]

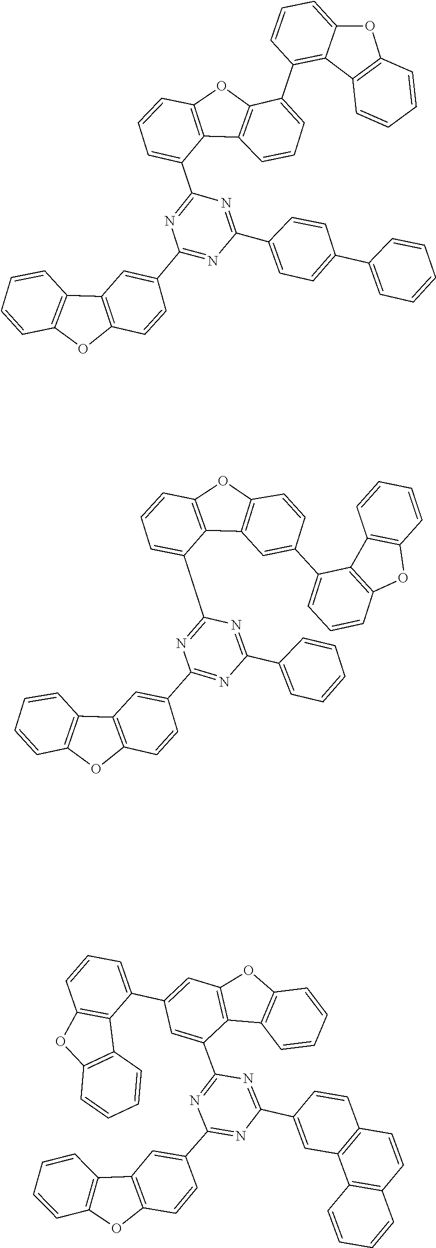 [SMILES: c1ccc(-c2ccc(-c3nc(-c4ccc5oc6ccccc6c5c4)nc(-c4cccc5oc6c(-c7cccc8oc9ccccc9c78)cccc6c45)n3)cc2)cc1 ; c1ccc(-c2nc(-c3ccc4oc5ccccc5c4c3)nc(-c3cccc4oc5ccc(-c6cccc7oc8ccccc8c67)cc5c34)n2)cc1 ; c1ccc2c(c1)ccc1ccc(-c3nc(-c4ccc5oc6ccccc6c5c4)nc(-c4cc(-c5cccc6oc7ccccc7c56)cc5oc6ccccc6c45)n3)cc12]

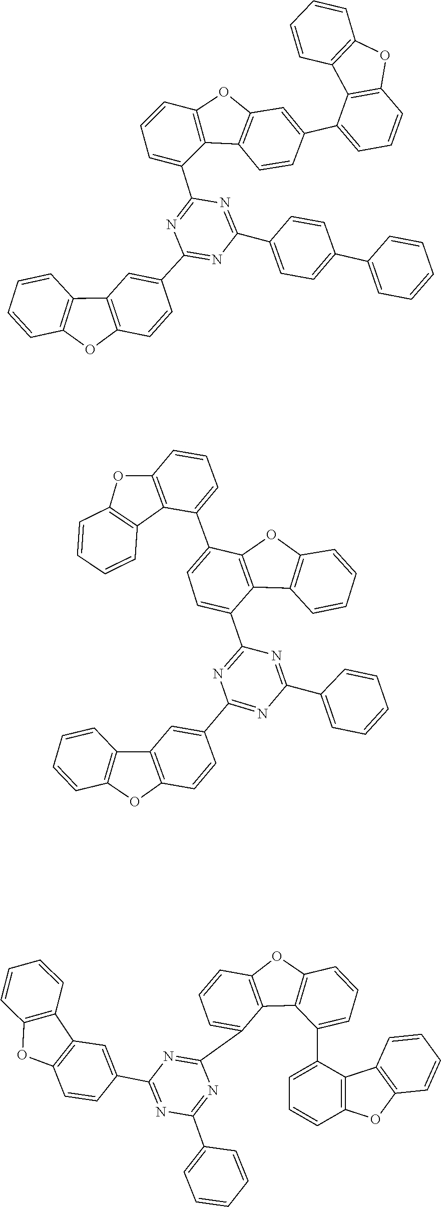 [SMILES: c1ccc(-c2ccc(-c3nc(-c4ccc5oc6ccccc6c5c4)nc(-c4cccc5oc6cc(-c7cccc8oc9ccccc9c78)ccc6c45)n3)cc2)cc1 ; c1ccc(-c2nc(-c3ccc4oc5ccccc5c4c3)nc(-c3ccc(-c4cccc5oc6ccccc6c45)c4oc5ccccc5c34)n2)cc1 ; c1ccc(-c2nc(-c3ccc4oc5ccccc5c4c3)nc(-c3cccc4oc5cccc(-c6cccc7oc8ccccc8c67)c5c34)n2)cc1]

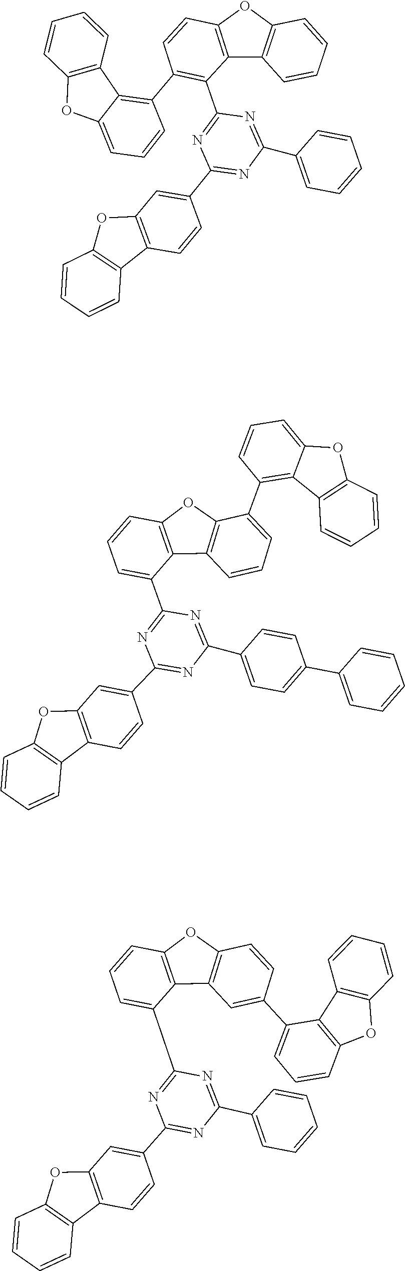 [SMILES: c1ccc(-c2nc(-c3ccc4c(c3)oc3ccccc34)nc(-c3c(-c4cccc5oc6ccccc6c45)ccc4oc5ccccc5c34)n2)cc1 ; c1ccc(-c2ccc(-c3nc(-c4ccc5c(c4)oc4ccccc45)nc(-c4cccc5oc6c(-c7cccc8oc9ccccc9c78)cccc6c45)n3)cc2)cc1 ; c1ccc(-c2nc(-c3ccc4c(c3)oc3ccccc34)nc(-c3cccc4oc5ccc(-c6cccc7oc8ccccc8c67)cc5c34)n2)cc1]

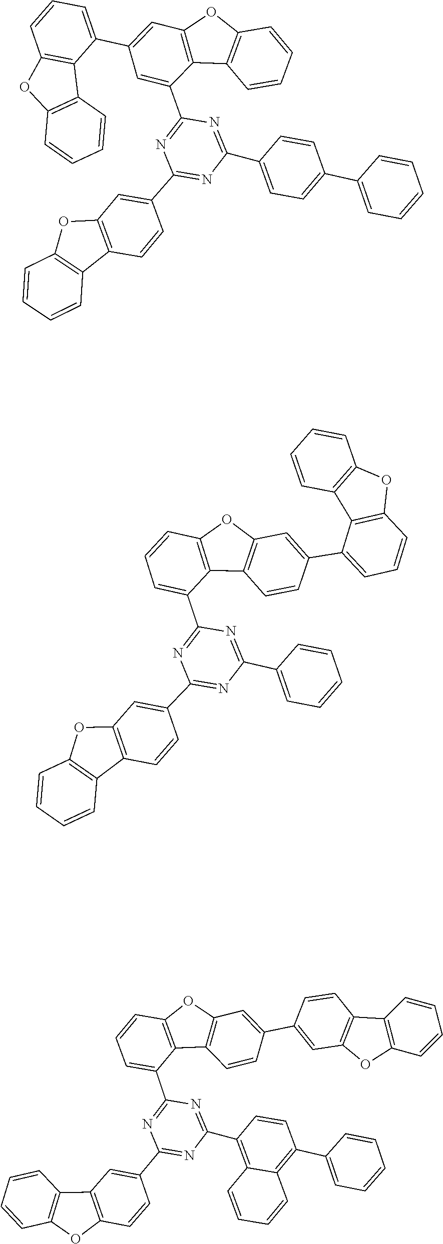 [SMILES: c1ccc(-c2ccc(-c3nc(-c4ccc5c(c4)oc4ccccc45)nc(-c4cc(-c5cccc6oc7ccccc7c56)cc5oc6ccccc6c45)n3)cc2)cc1 ; c1ccc(-c2nc(-c3ccc4c(c3)oc3ccccc34)nc(-c3cccc4oc5cc(-c6cccc7oc8ccccc8c67)ccc5c34)n2)cc1 ; c1ccc(-c2ccc(-c3nc(-c4ccc5oc6ccccc6c5c4)nc(-c4cccc5oc6cc(-c7ccc8c(c7)oc7ccccc78)ccc6c45)n3)c3ccccc23)cc1]

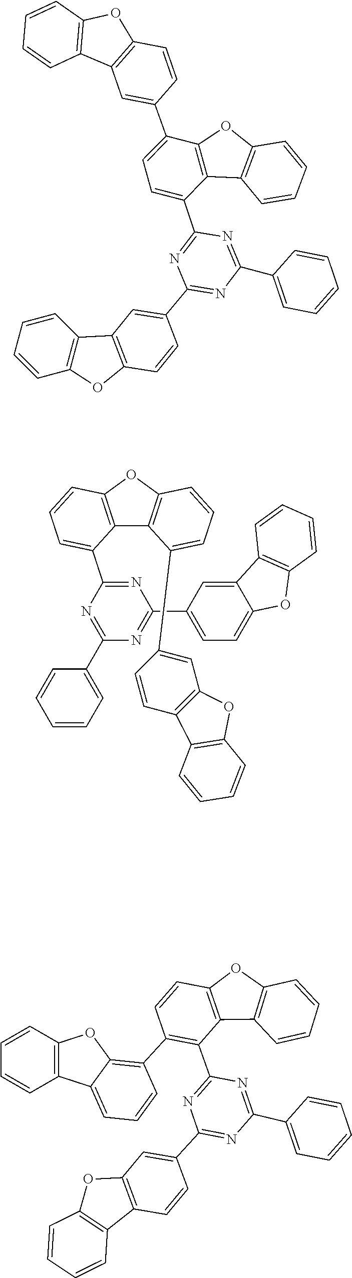 [SMILES: c1ccc(-c2nc(-c3ccc4oc5ccccc5c4c3)nc(-c3ccc(-c4ccc5oc6ccccc6c5c4)c4oc5ccccc5c34)n2)cc1 ; c1ccc(-c2nc(-c3ccc4oc5ccccc5c4c3)nc(-c3cccc4oc5cccc(-c6ccc7c(c6)oc6ccccc67)c5c34)n2)cc1 ; c1ccc(-c2nc(-c3ccc4c(c3)oc3ccccc34)nc(-c3c(-c4cccc5c4oc4ccccc45)ccc4oc5ccccc5c34)n2)cc1]

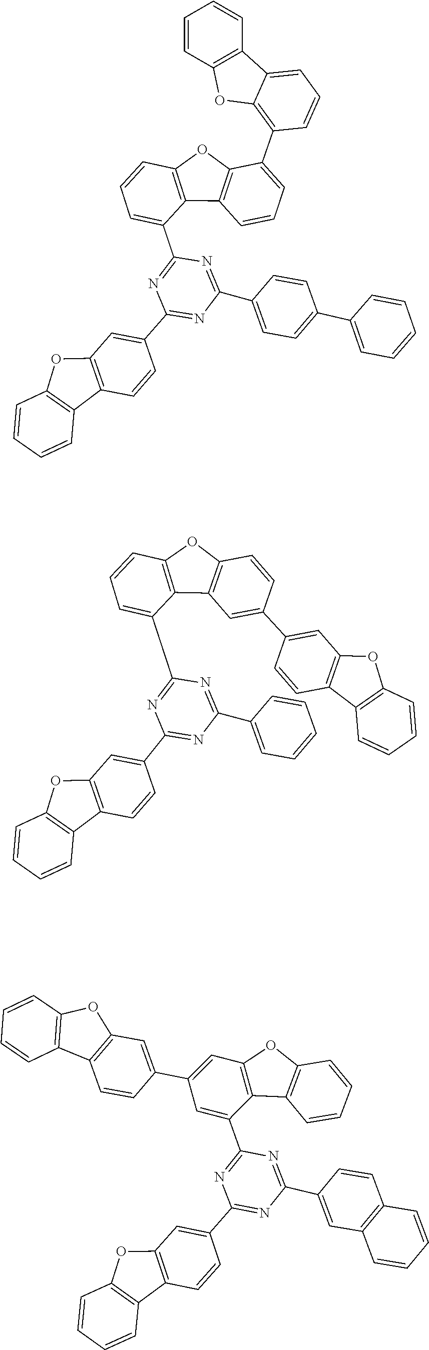 [SMILES: c1ccc(-c2ccc(-c3nc(-c4ccc5c(c4)oc4ccccc45)nc(-c4cccc5oc6c(-c7cccc8c7oc7ccccc78)cccc6c45)n3)cc2)cc1 ; c1ccc(-c2nc(-c3ccc4c(c3)oc3ccccc34)nc(-c3cccc4oc5ccc(-c6ccc7c(c6)oc6ccccc67)cc5c34)n2)cc1 ; c1ccc2cc(-c3nc(-c4ccc5c(c4)oc4ccccc45)nc(-c4cc(-c5ccc6c(c5)oc5ccccc56)cc5oc6ccccc6c45)n3)ccc2c1]

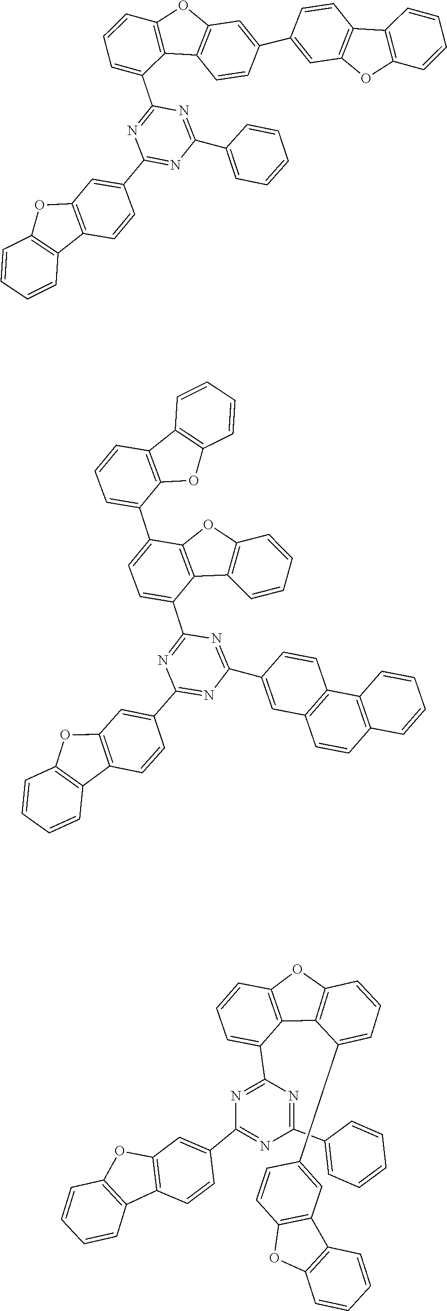 [SMILES: c1ccc(-c2nc(-c3ccc4c(c3)oc3ccccc34)nc(-c3cccc4oc5cc(-c6ccc7c(c6)oc6ccccc67)ccc5c34)n2)cc1 ; c1ccc2c(c1)ccc1cc(-c3nc(-c4ccc5c(c4)oc4ccccc45)nc(-c4ccc(-c5cccc6c5oc5ccccc56)c5oc6ccccc6c45)n3)ccc12 ; c1ccc(-c2nc(-c3ccc4c(c3)oc3ccccc34)nc(-c3cccc4oc5cccc(-c6ccc7oc8ccccc8c7c6)c5c34)n2)cc1]

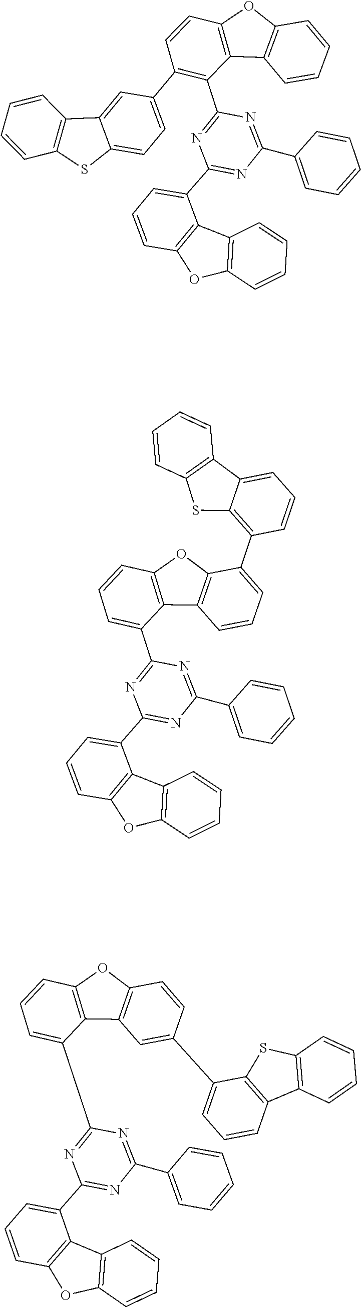 [SMILES: c1ccc(-c2nc(-c3cccc4oc5ccccc5c34)nc(-c3c(-c4ccc5sc6ccccc6c5c4)ccc4oc5ccccc5c34)n2)cc1 ; c1ccc(-c2nc(-c3cccc4oc5ccccc5c34)nc(-c3cccc4oc5c(-c6cccc7c6sc6ccccc67)cccc5c34)n2)cc1 ; c1ccc(-c2nc(-c3cccc4oc5ccccc5c34)nc(-c3cccc4oc5ccc(-c6cccc7c6sc6ccccc67)cc5c34)n2)cc1]

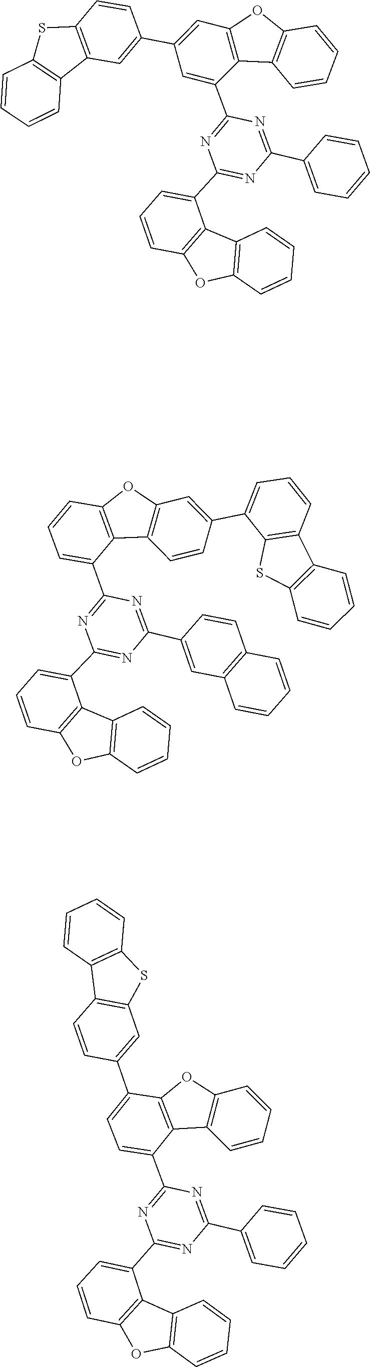 [SMILES: c1ccc(-c2nc(-c3cccc4oc5ccccc5c34)nc(-c3cc(-c4ccc5sc6ccccc6c5c4)cc4oc5ccccc5c34)n2)cc1 ; c1ccc2cc(-c3nc(-c4cccc5oc6ccccc6c45)nc(-c4cccc5oc6cc(-c7cccc8c7sc7ccccc78)ccc6c45)n3)ccc2c1 ; c1ccc(-c2nc(-c3cccc4oc5ccccc5c34)nc(-c3ccc(-c4ccc5c(c4)sc4ccccc45)c4oc5ccccc5c34)n2)cc1]

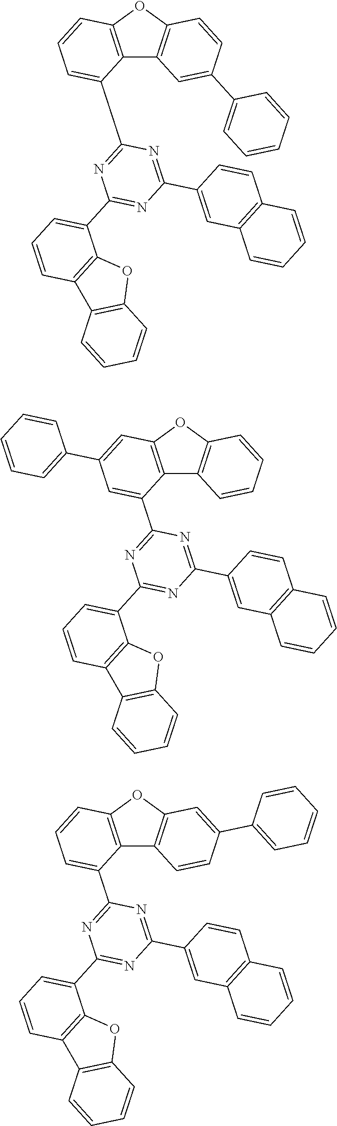

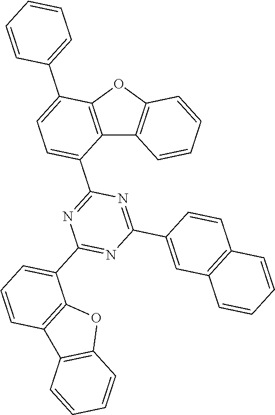 [SMILES: c1ccc(-c2ccc(-c3nc(-c4ccc5ccccc5c4)nc(-c4cccc5c4oc4ccccc45)n3)c3c2oc2ccccc23)cc1]

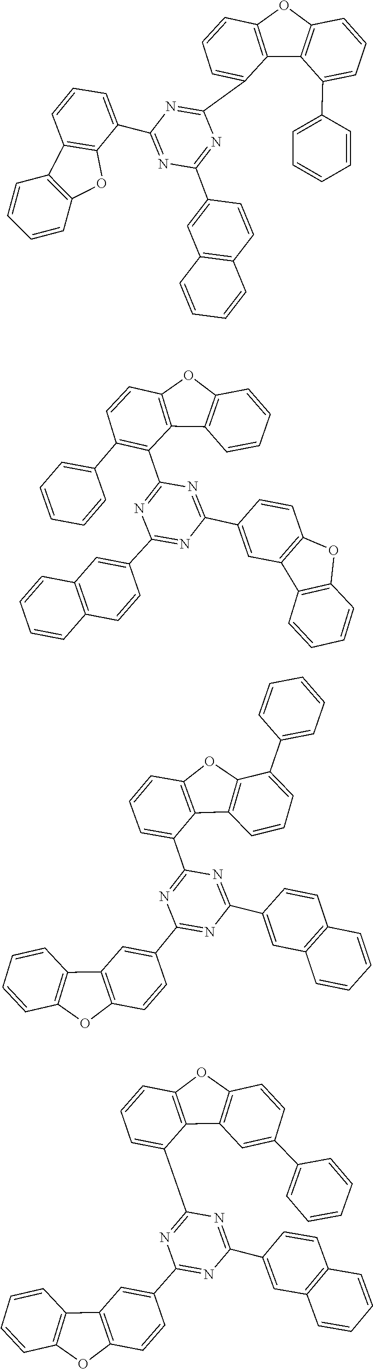

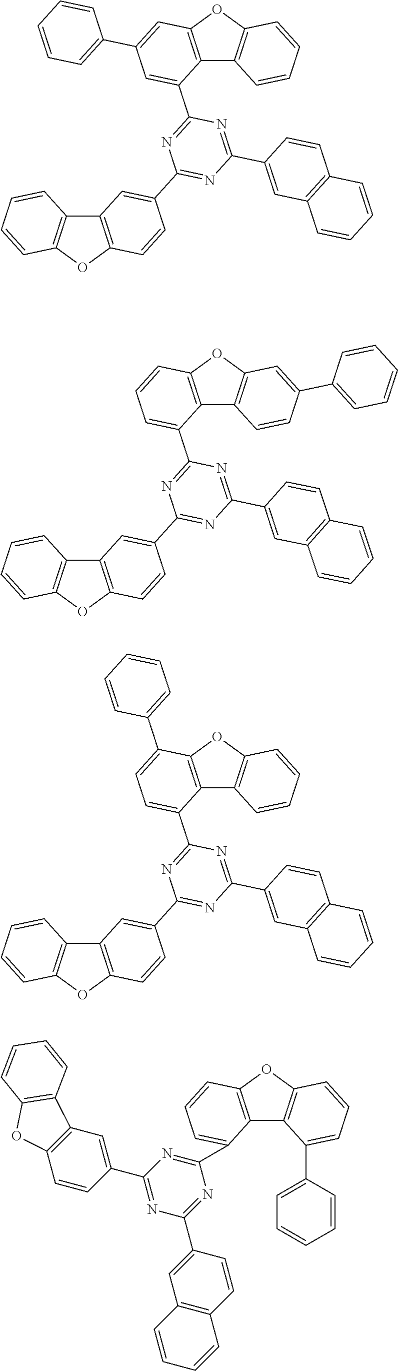 [SMILES: c1ccc(-c2cc(-c3nc(-c4ccc5ccccc5c4)nc(-c4ccc5oc6ccccc6c5c4)n3)c3c(c2)oc2ccccc23)cc1 ; c1ccc(-c2ccc3c(c2)oc2cccc(-c4nc(-c5ccc6ccccc6c5)nc(-c5ccc6oc7ccccc7c6c5)n4)c23)cc1 ; c1ccc(-c2ccc(-c3nc(-c4ccc5ccccc5c4)nc(-c4ccc5oc6ccccc6c5c4)n3)c3c2oc2ccccc23)cc1 ; c1ccc(-c2cccc3oc4cccc(-c5nc(-c6ccc7ccccc7c6)nc(-c6ccc7oc8ccccc8c7c6)n5)c4c23)cc1]

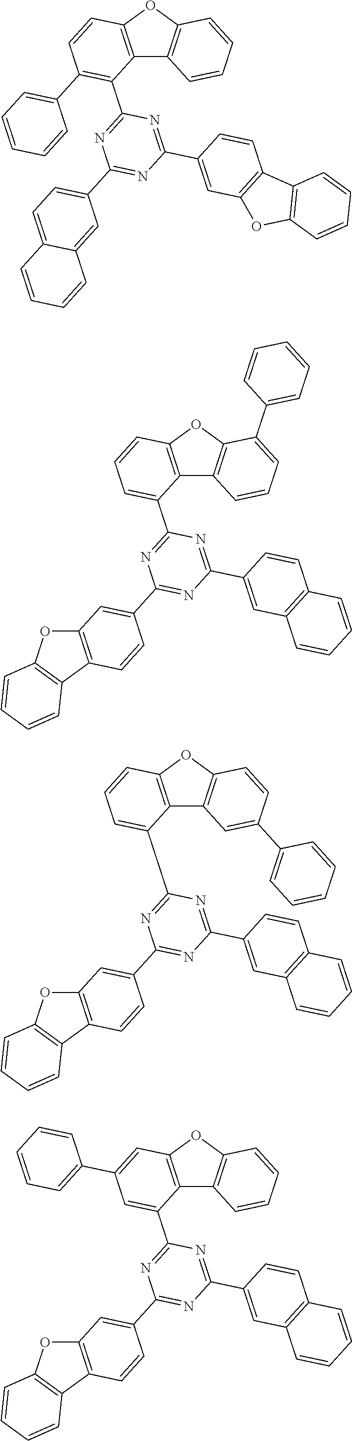

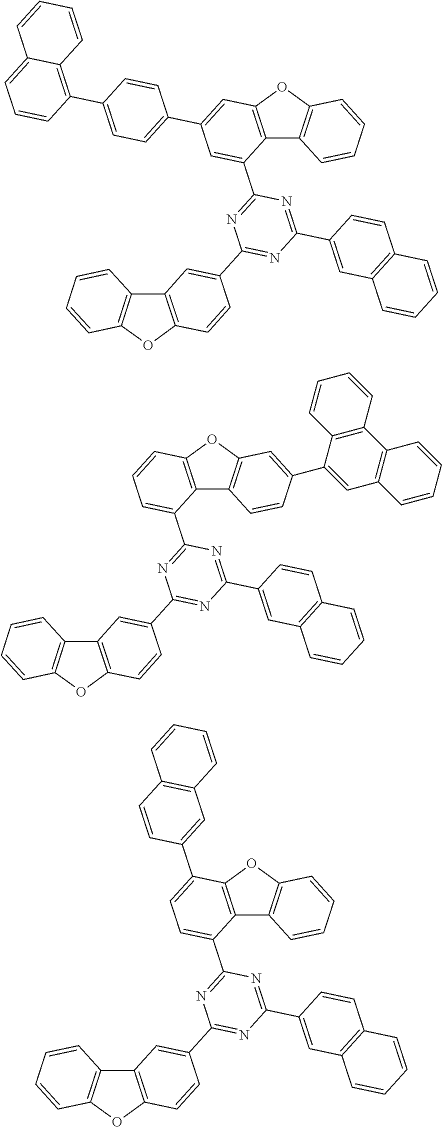 [SMILES: c1ccc2cc(-c3nc(-c4ccc5oc6ccccc6c5c4)nc(-c4cc(-c5ccc(-c6cccc7ccccc67)cc5)cc5oc6ccccc6c45)n3)ccc2c1 ; c1ccc2cc(-c3nc(-c4ccc5oc6ccccc6c5c4)nc(-c4cccc5oc6cc(-c7cc8ccccc8c8ccccc78)ccc6c45)n3)ccc2c1 ; c1ccc2cc(-c3nc(-c4ccc5oc6ccccc6c5c4)nc(-c4ccc(-c5ccc6ccccc6c5)c5oc6ccccc6c45)n3)ccc2c1]

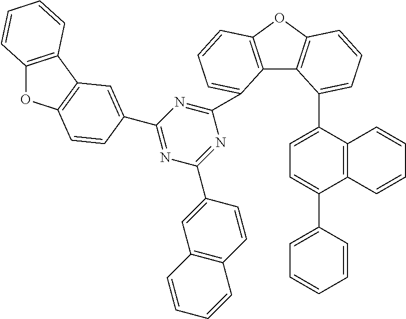 [SMILES: c1ccc(-c2ccc(-c3cccc4oc5cccc(-c6nc(-c7ccc8ccccc8c7)nc(-c7ccc8oc9ccccc9c8c7)n6)c5c34)c3ccccc23)cc1]

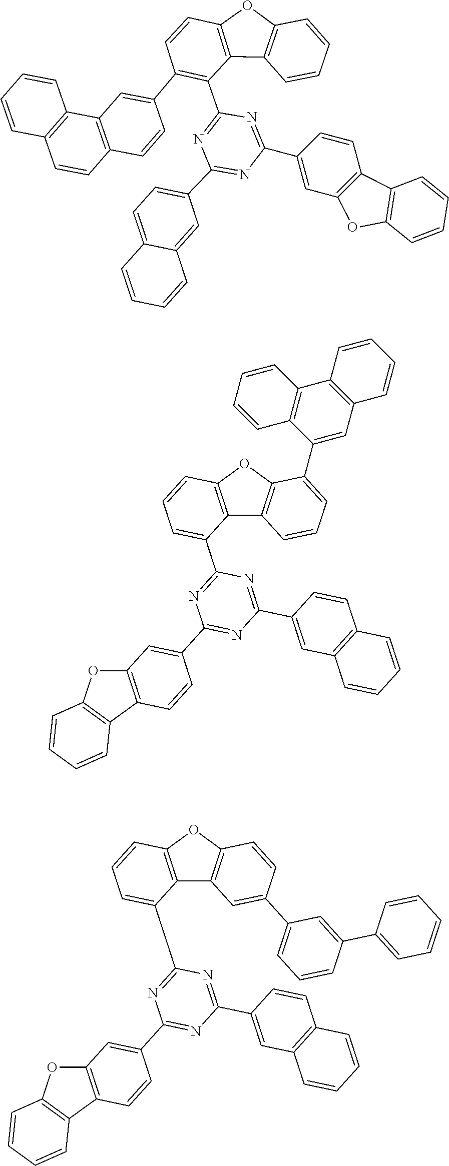 [SMILES: c1ccc2cc(-c3nc(-c4ccc5c(c4)oc4ccccc45)nc(-c4c(-c5ccc6ccc7ccccc7c6c5)ccc5oc6ccccc6c45)n3)ccc2c1 ; c1ccc2cc(-c3nc(-c4ccc5c(c4)oc4ccccc45)nc(-c4cccc5oc6c(-c7cc8ccccc8c8ccccc78)cccc6c45)n3)ccc2c1 ; c1ccc(-c2cccc(-c3ccc4oc5cccc(-c6nc(-c7ccc8ccccc8c7)nc(-c7ccc8c(c7)oc7ccccc78)n6)c5c4c3)c2)cc1]

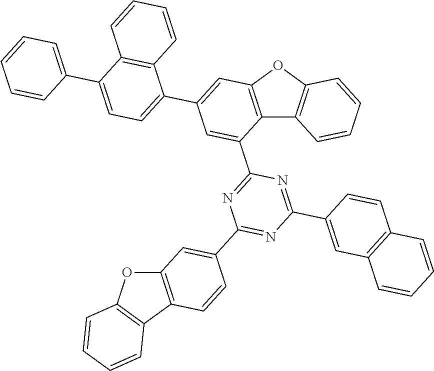 [SMILES: c1ccc(-c2ccc(-c3cc(-c4nc(-c5ccc6ccccc6c5)nc(-c5ccc6c(c5)oc5ccccc56)n4)c4c(c3)oc3ccccc34)c3ccccc23)cc1]

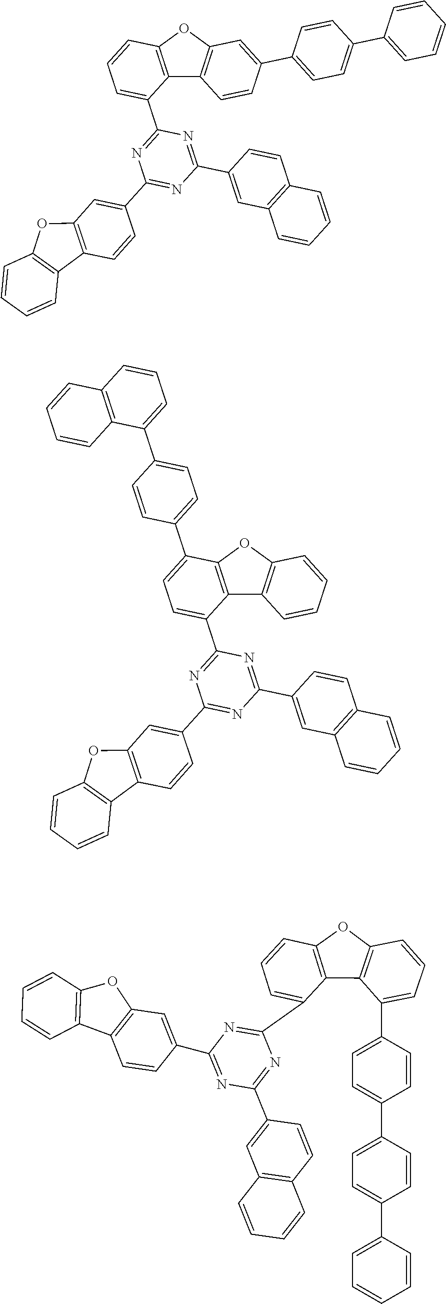 [SMILES: c1ccc(-c2ccc(-c3ccc4c(c3)oc3cccc(-c5nc(-c6ccc7ccccc7c6)nc(-c6ccc7c(c6)oc6ccccc67)n5)c34)cc2)cc1 ; c1ccc2cc(-c3nc(-c4ccc5c(c4)oc4ccccc45)nc(-c4ccc(-c5ccc(-c6cccc7ccccc67)cc5)c5oc6ccccc6c45)n3)ccc2c1 ; c1ccc(-c2ccc(-c3ccc(-c4cccc5oc6cccc(-c7nc(-c8ccc9ccccc9c8)nc(-c8ccc9c(c8)oc8ccccc89)n7)c6c45)cc3)cc2)cc1]

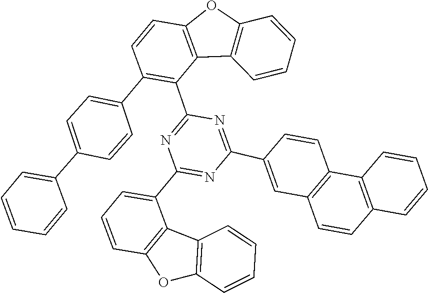 [SMILES: c1ccc(-c2ccc(-c3ccc4oc5ccccc5c4c3-c3nc(-c4ccc5c(ccc6ccccc65)c4)nc(-c4cccc5oc6ccccc6c45)n3)cc2)cc1]

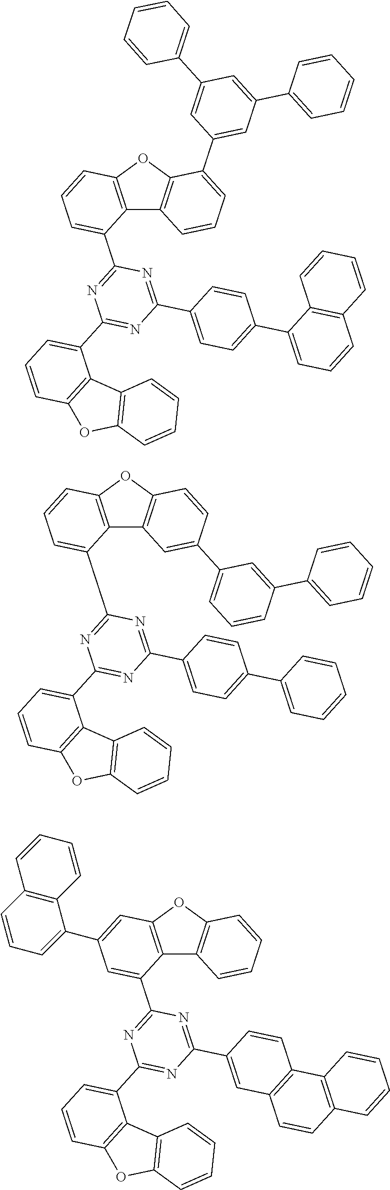

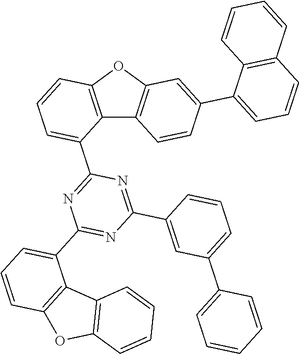 [SMILES: c1ccc(-c2cccc(-c3nc(-c4cccc5oc6ccccc6c45)nc(-c4cccc5oc6cc(-c7cccc8ccccc78)ccc6c45)n3)c2)cc1]

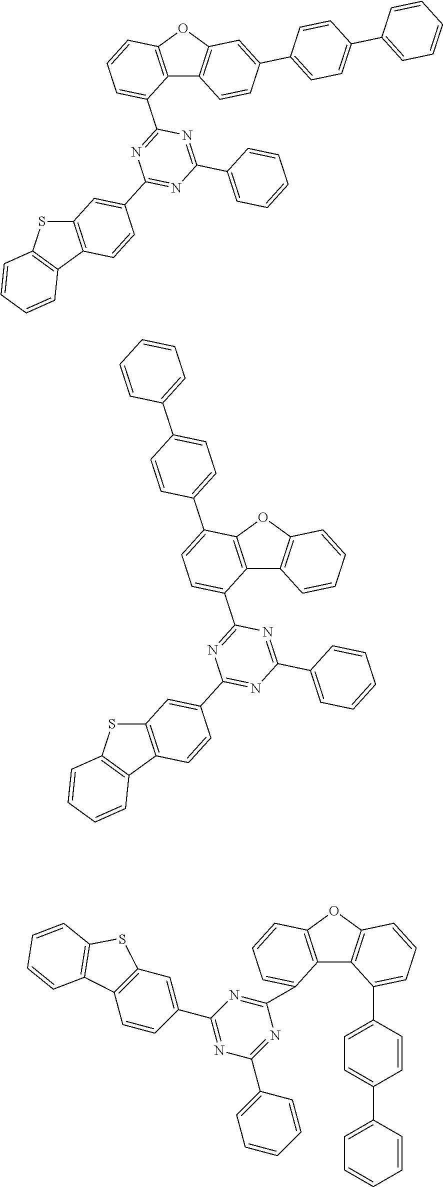 [SMILES: c1ccc(-c2ccc(-c3ccc4c(c3)oc3cccc(-c5nc(-c6ccccc6)nc(-c6ccc7c(c6)sc6ccccc67)n5)c34)cc2)cc1 ; c1ccc(-c2ccc(-c3ccc(-c4nc(-c5ccccc5)nc(-c5ccc6c(c5)sc5ccccc56)n4)c4c3oc3ccccc34)cc2)cc1 ; c1ccc(-c2ccc(-c3cccc4oc5cccc(-c6nc(-c7ccccc7)nc(-c7ccc8c(c7)sc7ccccc78)n6)c5c34)cc2)cc1]

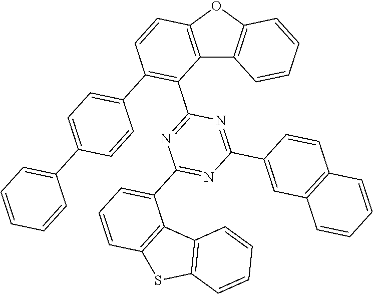 [SMILES: c1ccc(-c2ccc(-c3ccc4oc5ccccc5c4c3-c3nc(-c4ccc5ccccc5c4)nc(-c4cccc5sc6ccccc6c45)n3)cc2)cc1]

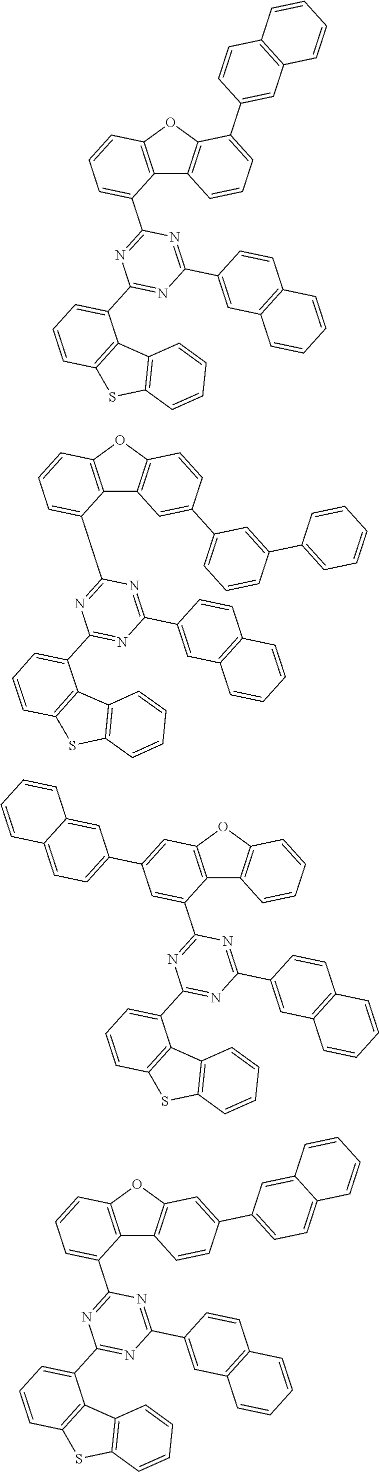

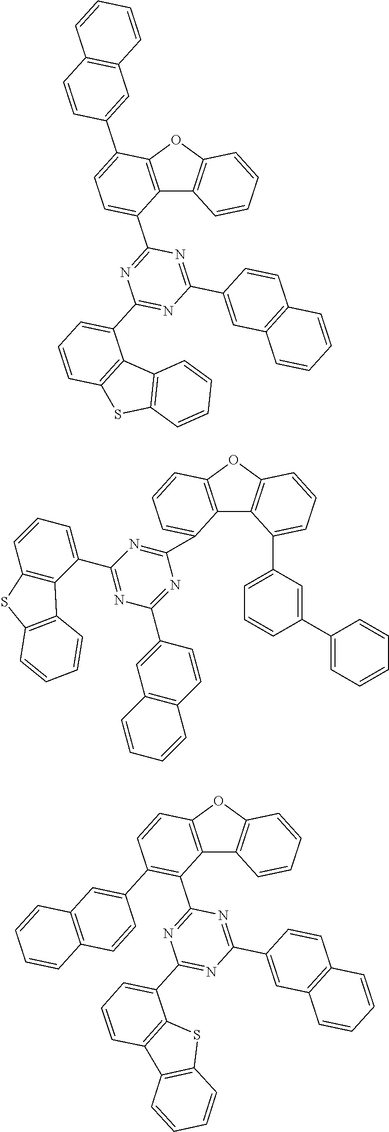

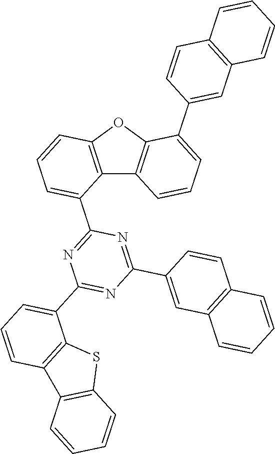 [SMILES: c1ccc2cc(-c3nc(-c4cccc5c4sc4ccccc45)nc(-c4cccc5oc6c(-c7ccc8ccccc8c7)cccc6c45)n3)ccc2c1]

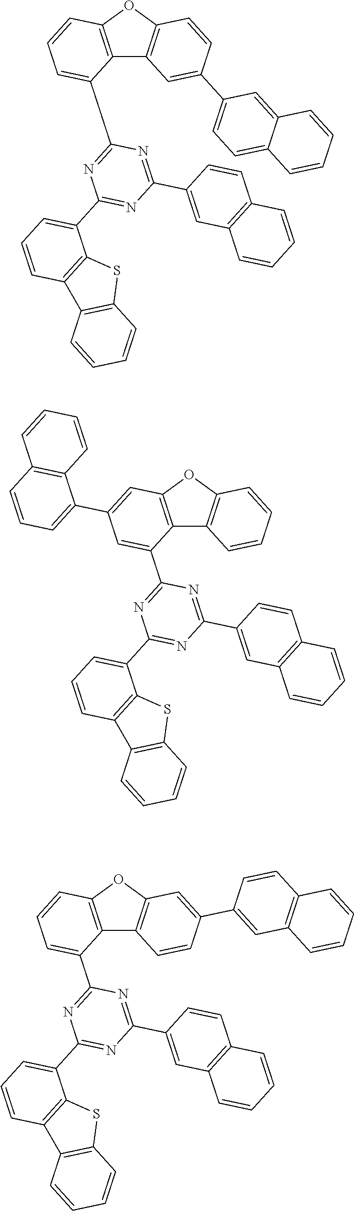 [SMILES: c1ccc2cc(-c3ccc4oc5cccc(-c6nc(-c7ccc8ccccc8c7)nc(-c7cccc8c7sc7ccccc78)n6)c5c4c3)ccc2c1 ; c1ccc2cc(-c3nc(-c4cccc5c4sc4ccccc45)nc(-c4cc(-c5cccc6ccccc56)cc5oc6ccccc6c45)n3)ccc2c1 ; c1ccc2cc(-c3ccc4c(c3)oc3cccc(-c5nc(-c6ccc7ccccc7c6)nc(-c6cccc7c6sc6ccccc67)n5)c34)ccc2c1]

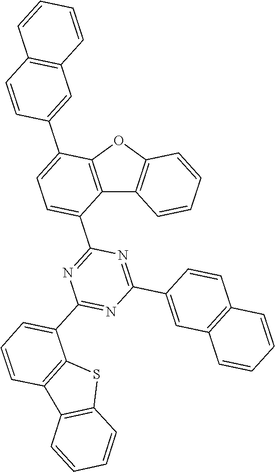 [SMILES: c1ccc2cc(-c3nc(-c4cccc5c4sc4ccccc45)nc(-c4ccc(-c5ccc6ccccc6c5)c5oc6ccccc6c45)n3)ccc2c1]

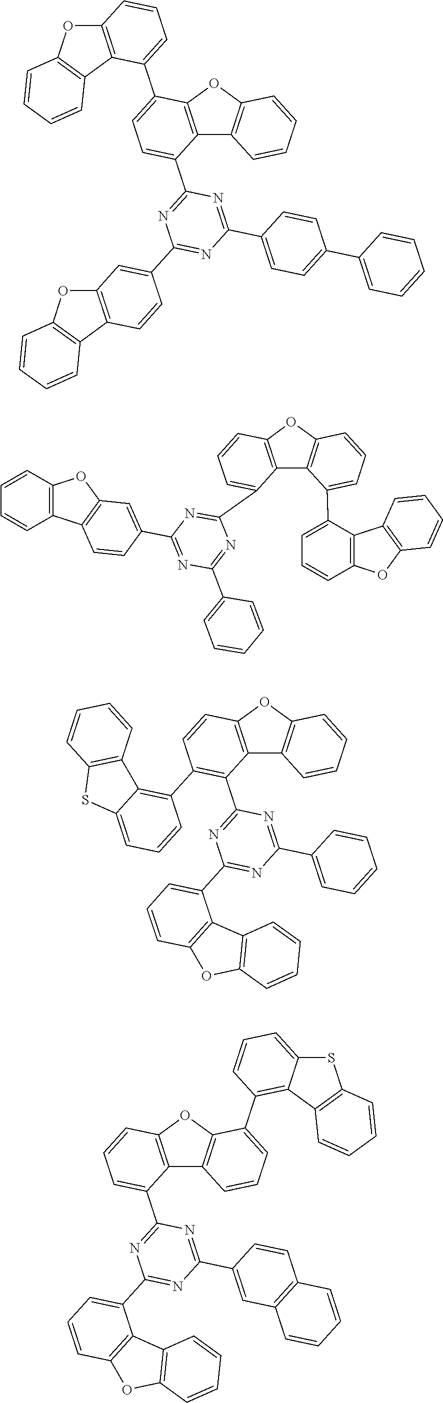

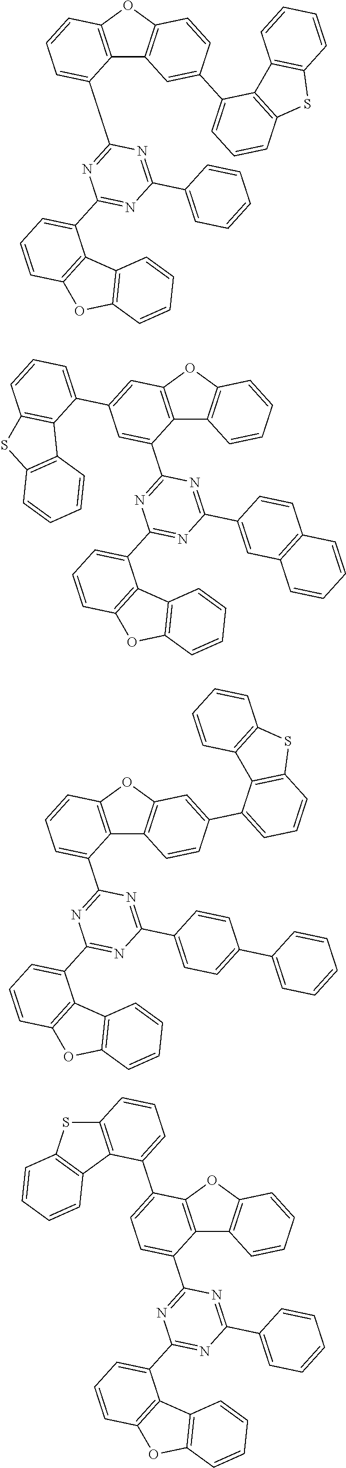

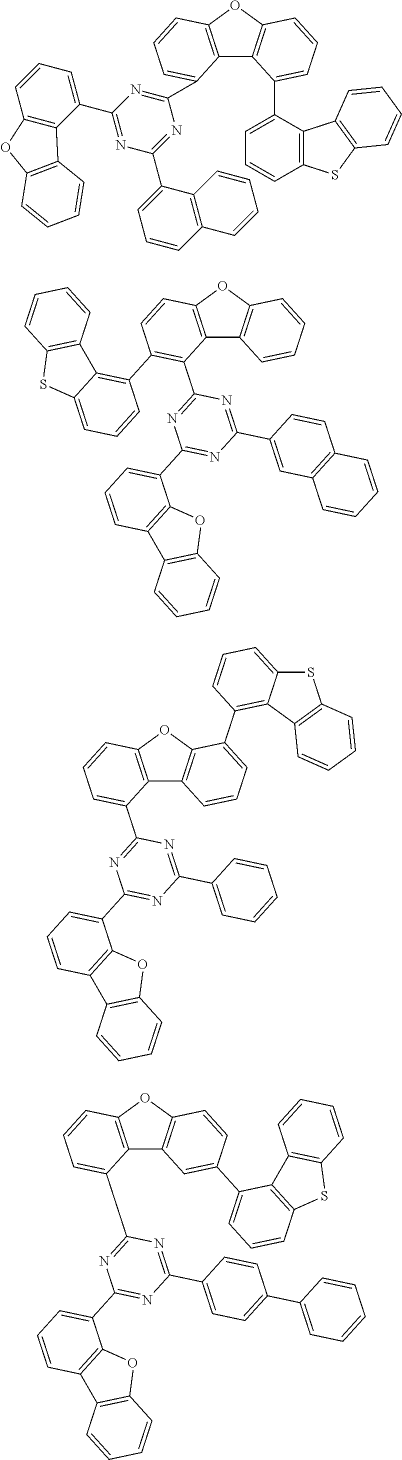

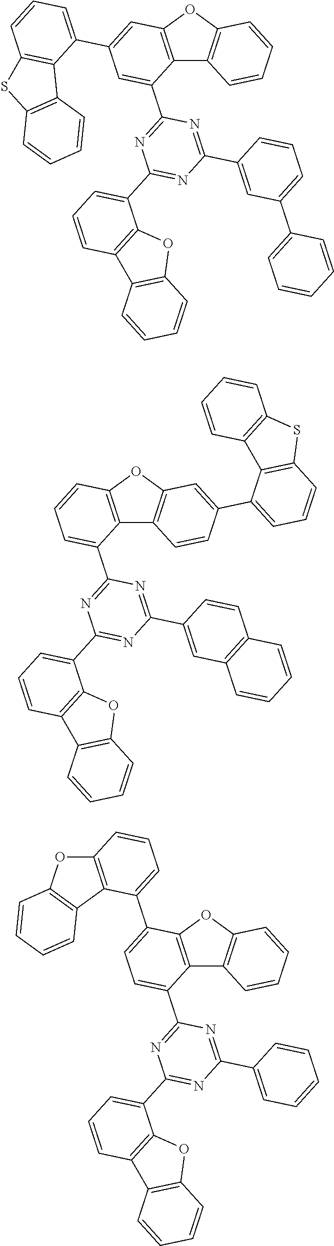

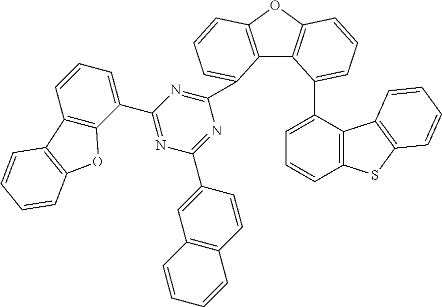 [SMILES: c1ccc2cc(-c3nc(-c4cccc5c4oc4ccccc45)nc(-c4cccc5oc6cccc(-c7cccc8sc9ccccc9c78)c6c45)n3)ccc2c1]

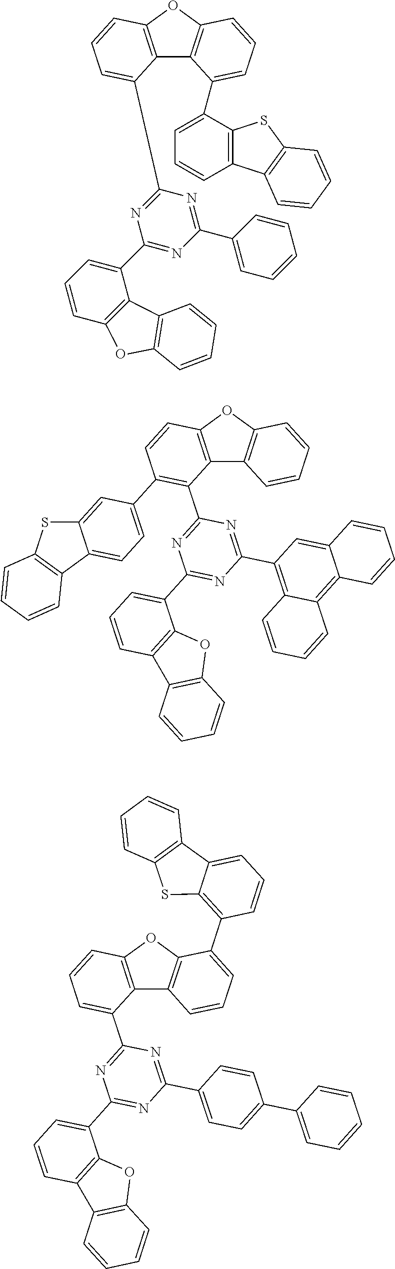 [SMILES: c1ccc(-c2nc(-c3cccc4oc5ccccc5c34)nc(-c3cccc4oc5cccc(-c6cccc7c6sc6ccccc67)c5c34)n2)cc1 ; c1ccc2c(c1)cc(-c1nc(-c3cccc4c3oc3ccccc34)nc(-c3c(-c4ccc5c(c4)sc4ccccc45)ccc4oc5ccccc5c34)n1)c1ccccc12 ; c1ccc(-c2ccc(-c3nc(-c4cccc5c4oc4ccccc45)nc(-c4cccc5oc6c(-c7cccc8c7sc7ccccc78)cccc6c45)n3)cc2)cc1]

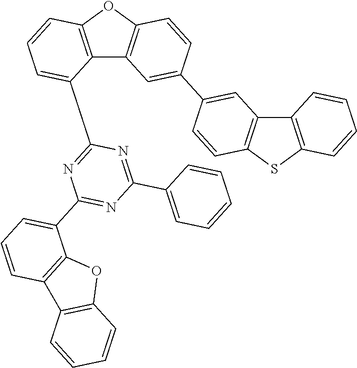 [SMILES: c1ccc(-c2nc(-c3cccc4c3oc3ccccc34)nc(-c3cccc4oc5ccc(-c6ccc7sc8ccccc8c7c6)cc5c34)n2)cc1]

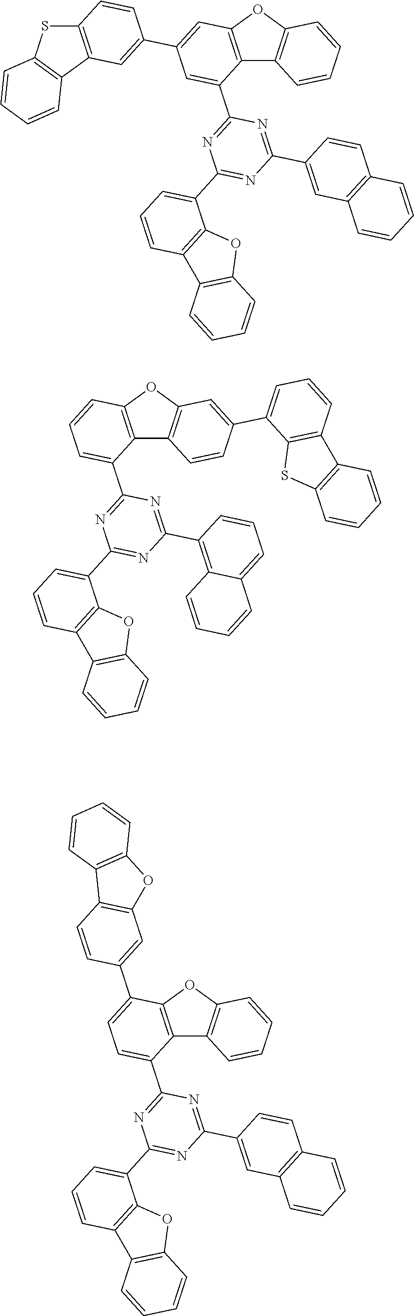 [SMILES: c1ccc2cc(-c3nc(-c4cccc5c4oc4ccccc45)nc(-c4cc(-c5ccc6sc7ccccc7c6c5)cc5oc6ccccc6c45)n3)ccc2c1 ; c1ccc2c(-c3nc(-c4cccc5c4oc4ccccc45)nc(-c4cccc5oc6cc(-c7cccc8c7sc7ccccc78)ccc6c45)n3)cccc2c1 ; c1ccc2cc(-c3nc(-c4cccc5c4oc4ccccc45)nc(-c4ccc(-c5ccc6c(c5)oc5ccccc56)c5oc6ccccc6c45)n3)ccc2c1]

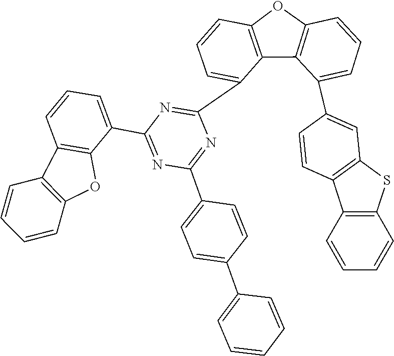 [SMILES: c1ccc(-c2ccc(-c3nc(-c4cccc5c4oc4ccccc45)nc(-c4cccc5oc6cccc(-c7ccc8c(c7)sc7ccccc78)c6c45)n3)cc2)cc1]

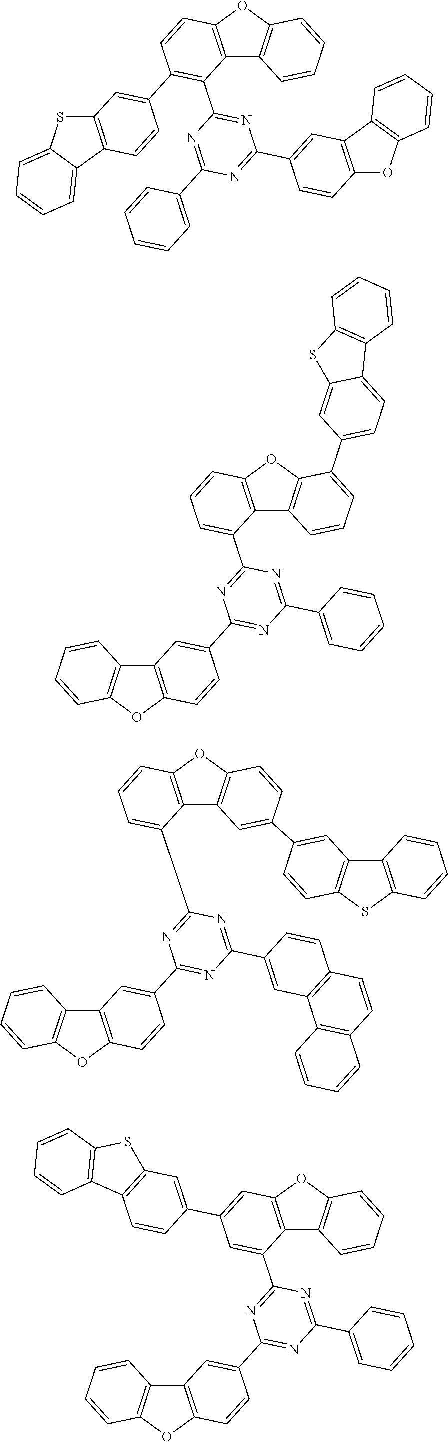 [SMILES: c1ccc(-c2nc(-c3ccc4oc5ccccc5c4c3)nc(-c3c(-c4ccc5c(c4)sc4ccccc45)ccc4oc5ccccc5c34)n2)cc1 ; c1ccc(-c2nc(-c3ccc4oc5ccccc5c4c3)nc(-c3cccc4oc5c(-c6ccc7c(c6)sc6ccccc67)cccc5c34)n2)cc1 ; c1ccc2c(c1)ccc1ccc(-c3nc(-c4ccc5oc6ccccc6c5c4)nc(-c4cccc5oc6ccc(-c7ccc8sc9ccccc9c8c7)cc6c45)n3)cc12 ; c1ccc(-c2nc(-c3ccc4oc5ccccc5c4c3)nc(-c3cc(-c4ccc5c(c4)sc4ccccc45)cc4oc5ccccc5c34)n2)cc1]

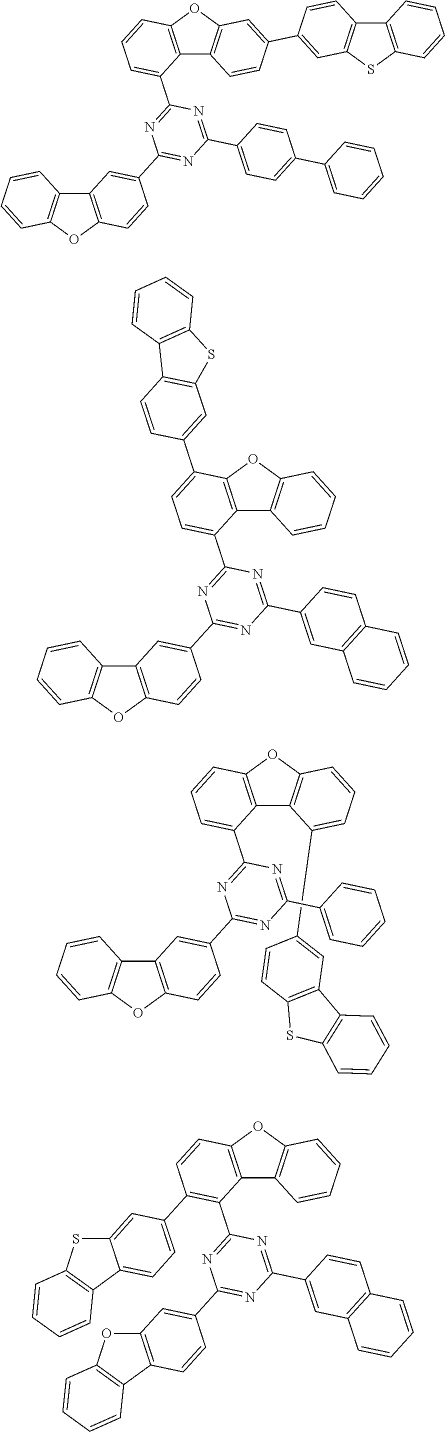 [SMILES: c1ccc(-c2ccc(-c3nc(-c4ccc5oc6ccccc6c5c4)nc(-c4cccc5oc6cc(-c7ccc8c(c7)sc7ccccc78)ccc6c45)n3)cc2)cc1 ; c1ccc2cc(-c3nc(-c4ccc5oc6ccccc6c5c4)nc(-c4ccc(-c5ccc6c(c5)sc5ccccc56)c5oc6ccccc6c45)n3)ccc2c1 ; c1ccc(-c2nc(-c3ccc4oc5ccccc5c4c3)nc(-c3cccc4oc5cccc(-c6ccc7sc8ccccc8c7c6)c5c34)n2)cc1 ; c1ccc2cc(-c3nc(-c4ccc5c(c4)oc4ccccc45)nc(-c4c(-c5ccc6c(c5)sc5ccccc56)ccc5oc6ccccc6c45)n3)ccc2c1]

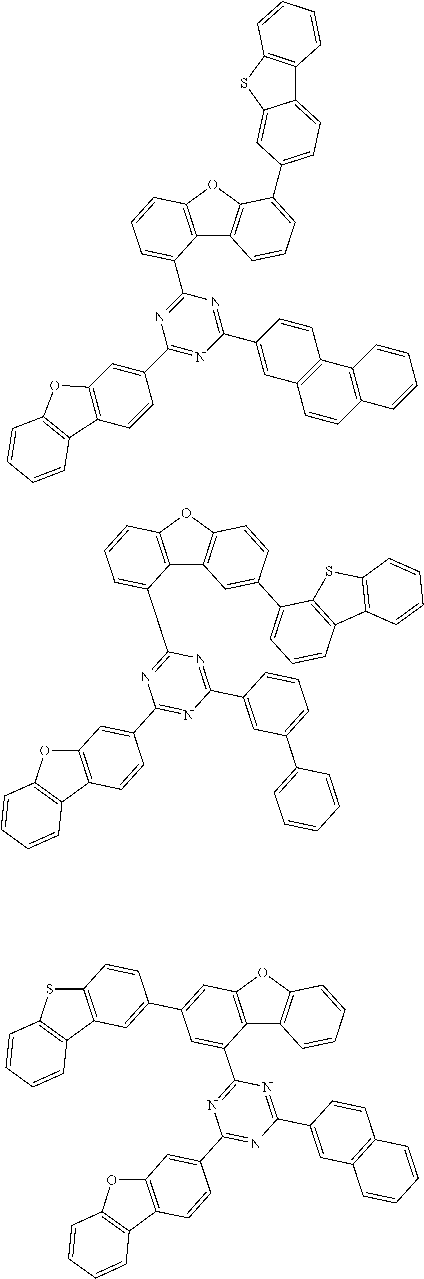 [SMILES: c1ccc2c(c1)ccc1cc(-c3nc(-c4ccc5c(c4)oc4ccccc45)nc(-c4cccc5oc6c(-c7ccc8c(c7)sc7ccccc78)cccc6c45)n3)ccc12 ; c1ccc(-c2cccc(-c3nc(-c4ccc5c(c4)oc4ccccc45)nc(-c4cccc5oc6ccc(-c7cccc8c7sc7ccccc78)cc6c45)n3)c2)cc1 ; c1ccc2cc(-c3nc(-c4ccc5c(c4)oc4ccccc45)nc(-c4cc(-c5ccc6sc7ccccc7c6c5)cc5oc6ccccc6c45)n3)ccc2c1]

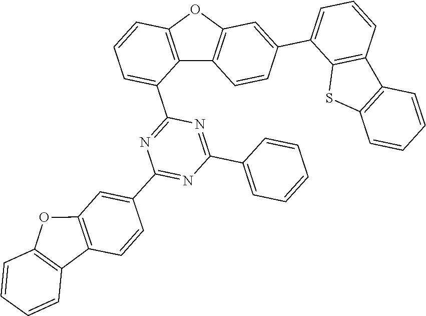 [SMILES: c1ccc(-c2nc(-c3ccc4c(c3)oc3ccccc34)nc(-c3cccc4oc5cc(-c6cccc7c6sc6ccccc67)ccc5c34)n2)cc1]

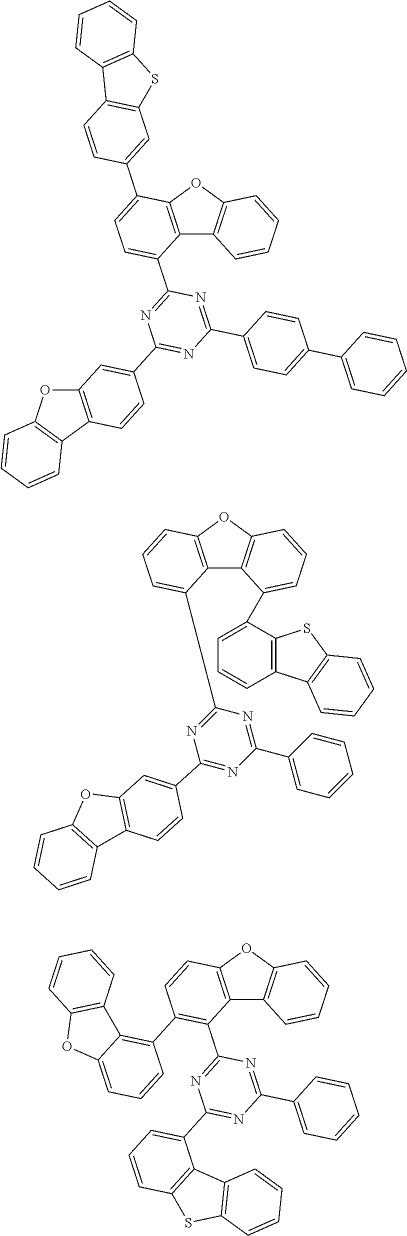 [SMILES: c1ccc(-c2ccc(-c3nc(-c4ccc5c(c4)oc4ccccc45)nc(-c4ccc(-c5ccc6c(c5)sc5ccccc56)c5oc6ccccc6c45)n3)cc2)cc1 ; c1ccc(-c2nc(-c3ccc4c(c3)oc3ccccc34)nc(-c3cccc4oc5cccc(-c6cccc7c6sc6ccccc67)c5c34)n2)cc1 ; c1ccc(-c2nc(-c3c(-c4cccc5oc6ccccc6c45)ccc4oc5ccccc5c34)nc(-c3cccc4sc5ccccc5c34)n2)cc1]

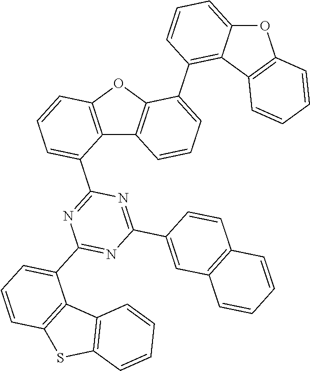 [SMILES: c1ccc2cc(-c3nc(-c4cccc5oc6c(-c7cccc8oc9ccccc9c78)cccc6c45)nc(-c4cccc5sc6ccccc6c45)n3)ccc2c1]

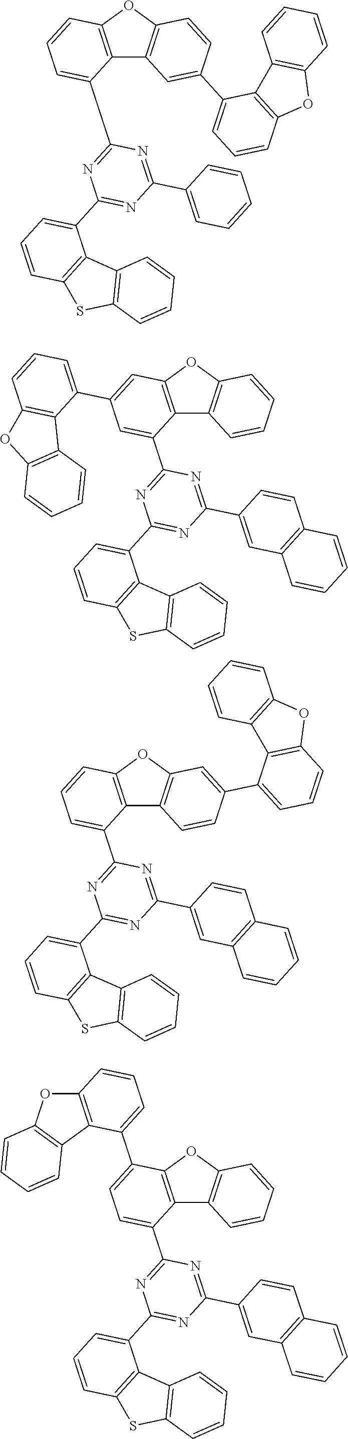

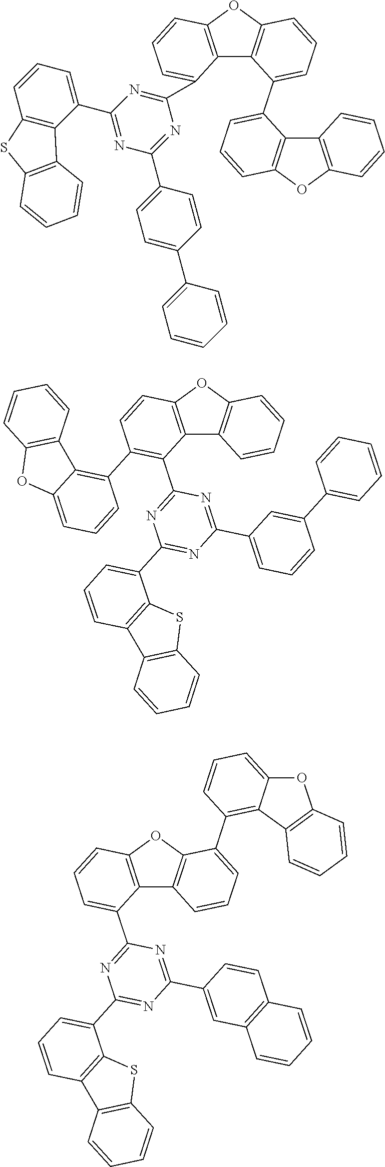

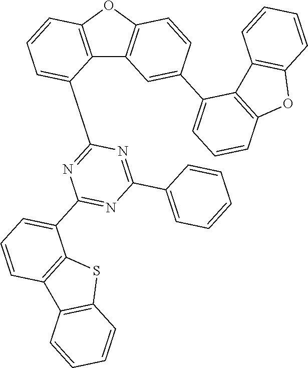 [SMILES: c1ccc(-c2nc(-c3cccc4c3sc3ccccc34)nc(-c3cccc4oc5ccc(-c6cccc7oc8ccccc8c67)cc5c34)n2)cc1]

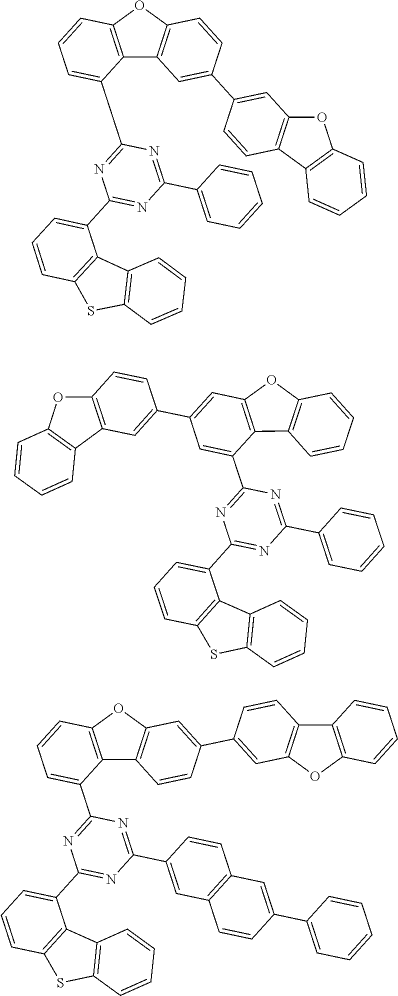

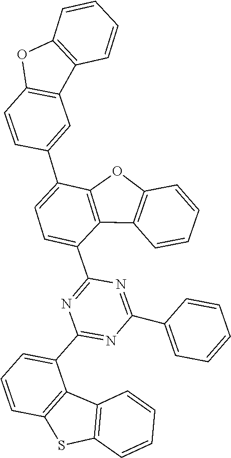 [SMILES: c1ccc(-c2nc(-c3cccc4sc5ccccc5c34)nc(-c3ccc(-c4ccc5oc6ccccc6c5c4)c4oc5ccccc5c34)n2)cc1]

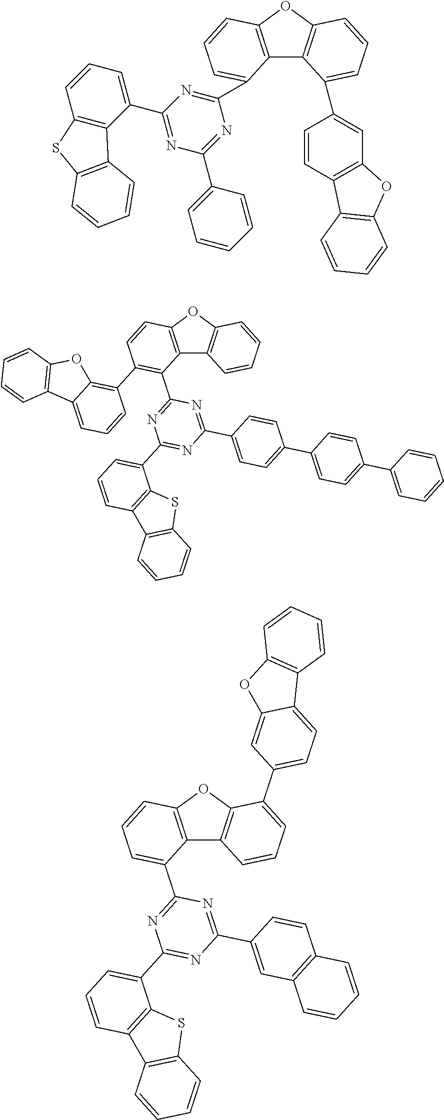 [SMILES: c1ccc(-c2nc(-c3cccc4sc5ccccc5c34)nc(-c3cccc4oc5cccc(-c6ccc7c(c6)oc6ccccc67)c5c34)n2)cc1 ; c1ccc(-c2ccc(-c3ccc(-c4nc(-c5cccc6c5sc5ccccc56)nc(-c5c(-c6cccc7c6oc6ccccc67)ccc6oc7ccccc7c56)n4)cc3)cc2)cc1 ; c1ccc2cc(-c3nc(-c4cccc5c4sc4ccccc45)nc(-c4cccc5oc6c(-c7ccc8c(c7)oc7ccccc78)cccc6c45)n3)ccc2c1]

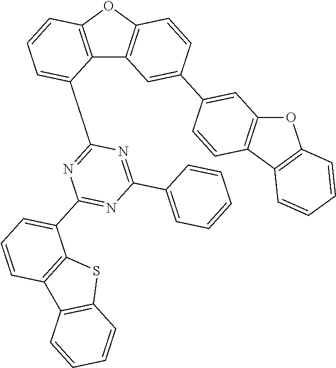 [SMILES: c1ccc(-c2nc(-c3cccc4c3sc3ccccc34)nc(-c3cccc4oc5ccc(-c6ccc7c(c6)oc6ccccc67)cc5c34)n2)cc1]

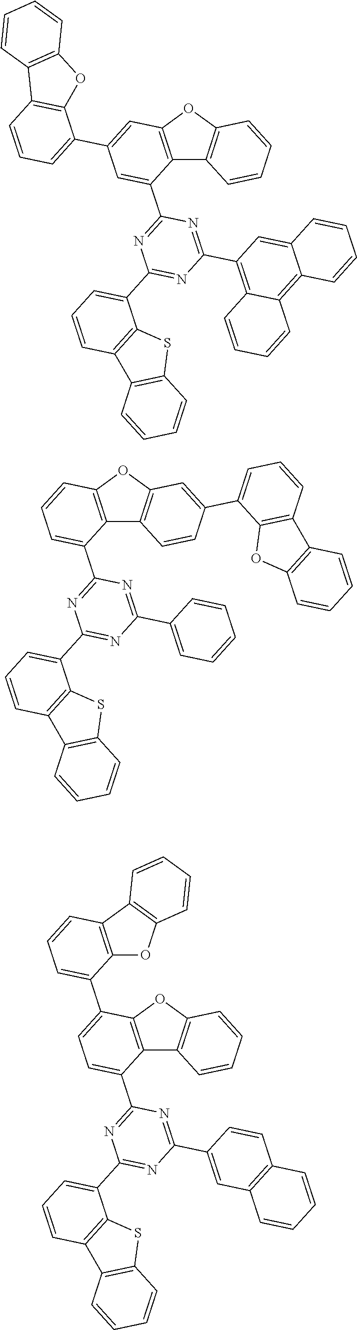

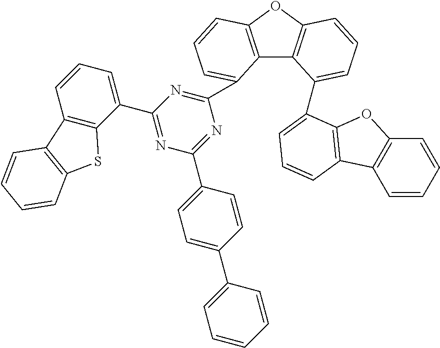 [SMILES: c1ccc(-c2ccc(-c3nc(-c4cccc5c4sc4ccccc45)nc(-c4cccc5oc6cccc(-c7cccc8c7oc7ccccc78)c6c45)n3)cc2)cc1]

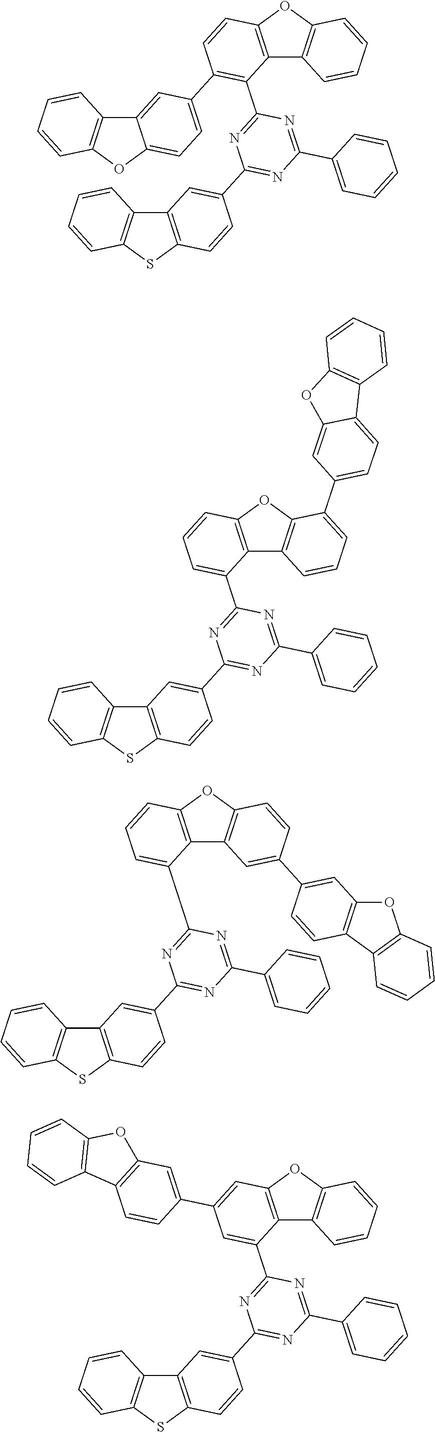

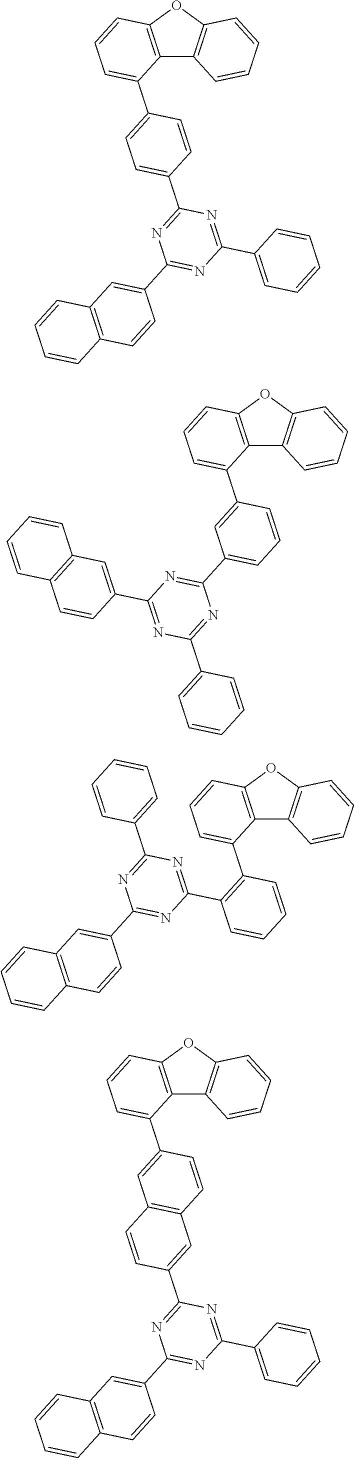 [SMILES: c1ccc(-c2nc(-c3ccc(-c4cccc5oc6ccccc6c45)cc3)nc(-c3ccc4ccccc4c3)n2)cc1 ; c1ccc(-c2nc(-c3cccc(-c4cccc5oc6ccccc6c45)c3)nc(-c3ccc4ccccc4c3)n2)cc1 ; c1ccc(-c2nc(-c3ccc4ccccc4c3)nc(-c3ccccc3-c3cccc4oc5ccccc5c34)n2)cc1 ; c1ccc(-c2nc(-c3ccc4ccccc4c3)nc(-c3ccc4cc(-c5cccc6oc7ccccc7c56)ccc4c3)n2)cc1]

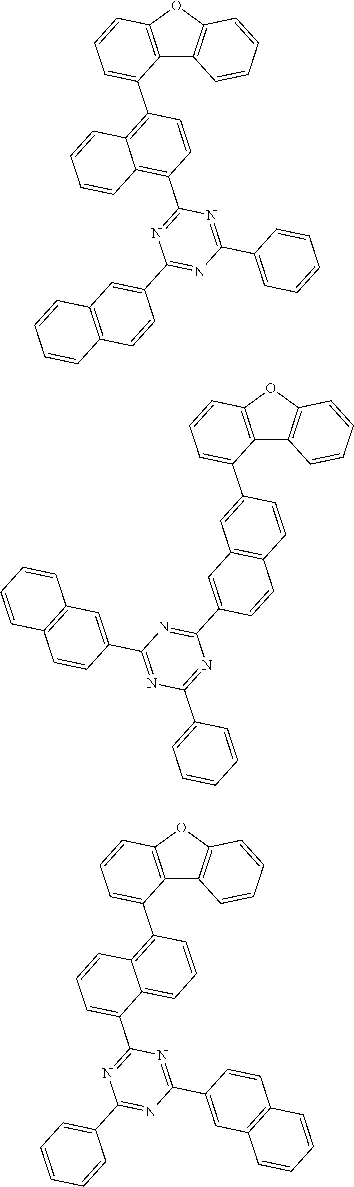 [SMILES: c1ccc(-c2nc(-c3ccc4ccccc4c3)nc(-c3ccc(-c4cccc5oc6ccccc6c45)c4ccccc34)n2)cc1 ; c1ccc(-c2nc(-c3ccc4ccccc4c3)nc(-c3ccc4ccc(-c5cccc6oc7ccccc7c56)cc4c3)n2)cc1 ; c1ccc(-c2nc(-c3ccc4ccccc4c3)nc(-c3cccc4c(-c5cccc6oc7ccccc7c56)cccc34)n2)cc1]

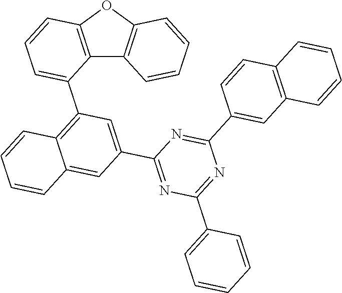 [SMILES: c1ccc(-c2nc(-c3ccc4ccccc4c3)nc(-c3cc(-c4cccc5oc6ccccc6c45)c4ccccc4c3)n2)cc1]

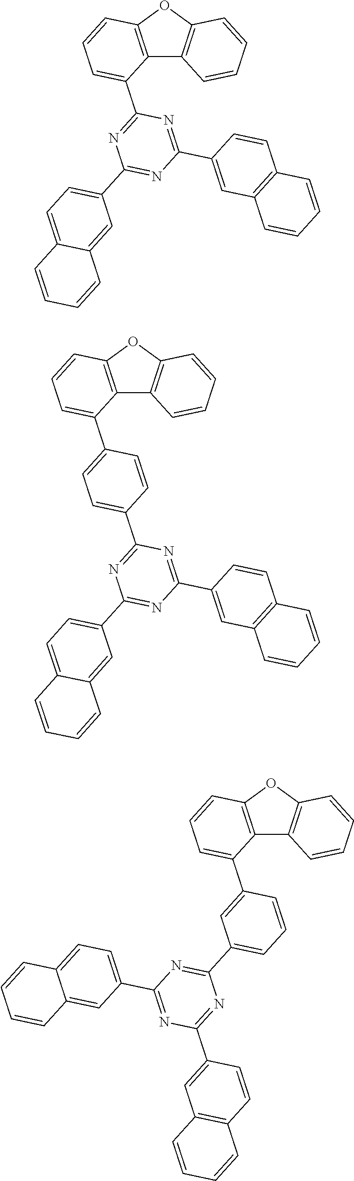 [SMILES: c1ccc2cc(-c3nc(-c4ccc5ccccc5c4)nc(-c4cccc5oc6ccccc6c45)n3)ccc2c1 ; c1ccc2cc(-c3nc(-c4ccc(-c5cccc6oc7ccccc7c56)cc4)nc(-c4ccc5ccccc5c4)n3)ccc2c1 ; c1cc(-c2nc(-c3ccc4ccccc4c3)nc(-c3ccc4ccccc4c3)n2)cc(-c2cccc3oc4ccccc4c23)c1]

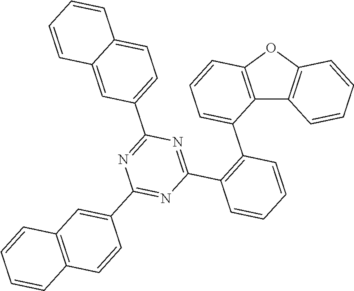 [SMILES: c1ccc(-c2cccc3oc4ccccc4c23)c(-c2nc(-c3ccc4ccccc4c3)nc(-c3ccc4ccccc4c3)n2)c1]

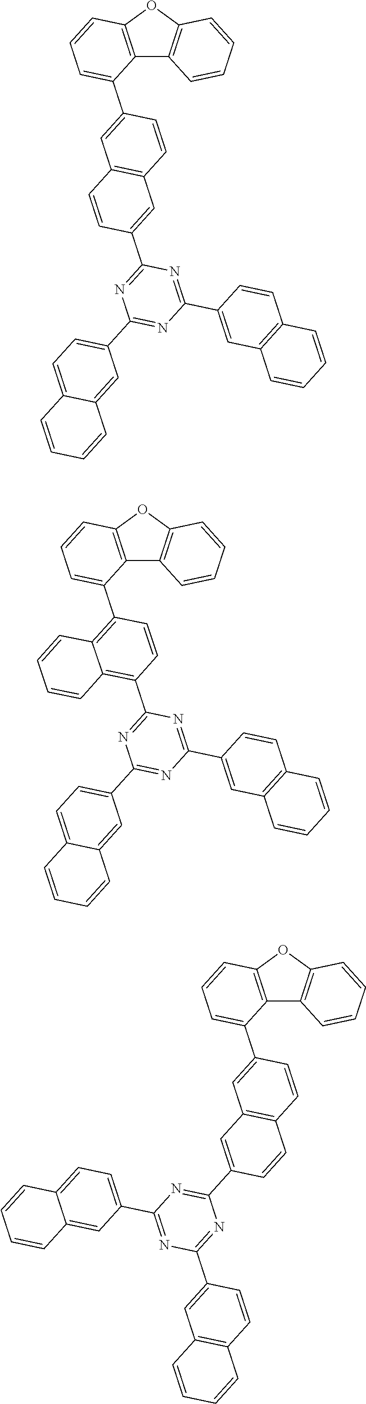 [SMILES: c1ccc2cc(-c3nc(-c4ccc5ccccc5c4)nc(-c4ccc5cc(-c6cccc7oc8ccccc8c67)ccc5c4)n3)ccc2c1 ; c1ccc2cc(-c3nc(-c4ccc5ccccc5c4)nc(-c4ccc(-c5cccc6oc7ccccc7c56)c5ccccc45)n3)ccc2c1 ; c1ccc2cc(-c3nc(-c4ccc5ccccc5c4)nc(-c4ccc5ccc(-c6cccc7oc8ccccc8c67)cc5c4)n3)ccc2c1]

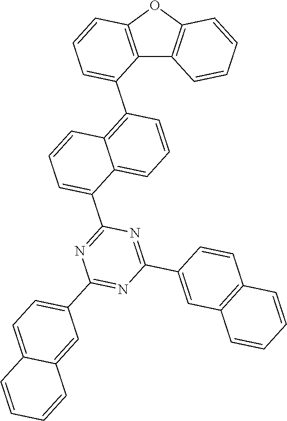 [SMILES: c1ccc2cc(-c3nc(-c4ccc5ccccc5c4)nc(-c4cccc5c(-c6cccc7oc8ccccc8c67)cccc45)n3)ccc2c1]

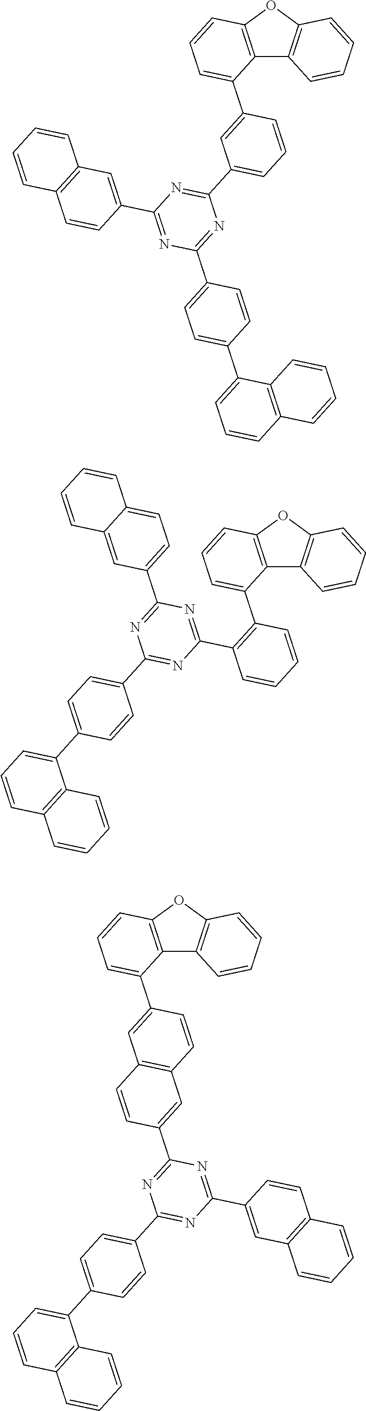 [SMILES: c1cc(-c2nc(-c3ccc(-c4cccc5ccccc45)cc3)nc(-c3ccc4ccccc4c3)n2)cc(-c2cccc3oc4ccccc4c23)c1 ; c1ccc(-c2cccc3oc4ccccc4c23)c(-c2nc(-c3ccc(-c4cccc5ccccc45)cc3)nc(-c3ccc4ccccc4c3)n2)c1 ; c1ccc2cc(-c3nc(-c4ccc(-c5cccc6ccccc56)cc4)nc(-c4ccc5cc(-c6cccc7oc8ccccc8c67)ccc5c4)n3)ccc2c1]

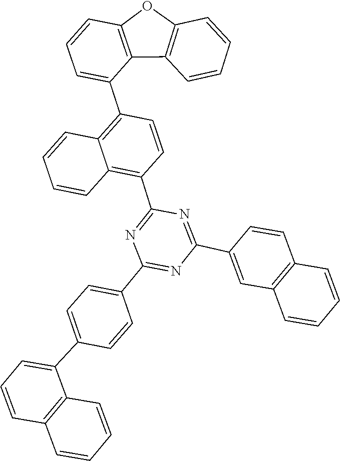 [SMILES: c1ccc2cc(-c3nc(-c4ccc(-c5cccc6ccccc56)cc4)nc(-c4ccc(-c5cccc6oc7ccccc7c56)c5ccccc45)n3)ccc2c1]

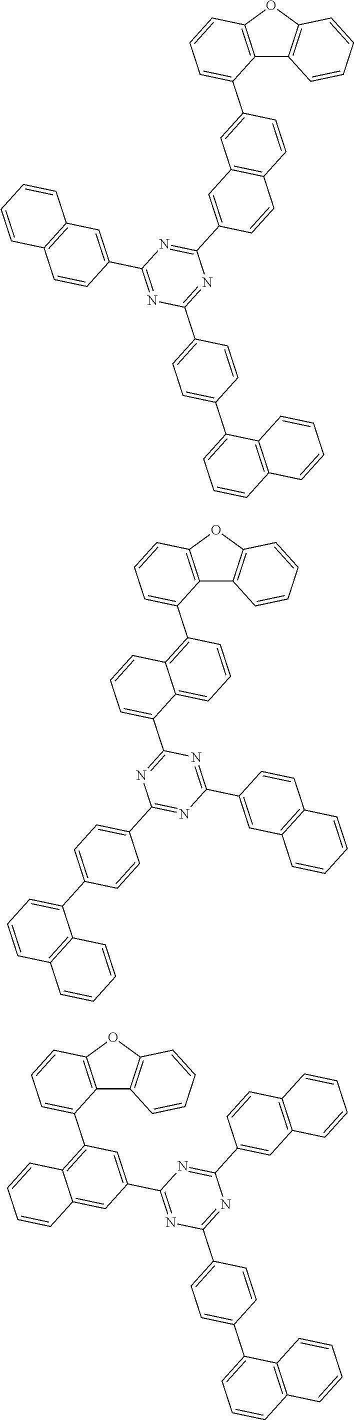

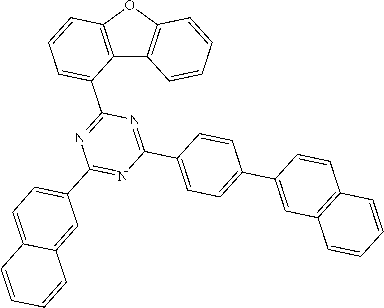 [SMILES: c1ccc2cc(-c3ccc(-c4nc(-c5ccc6ccccc6c5)nc(-c5cccc6oc7ccccc7c56)n4)cc3)ccc2c1]

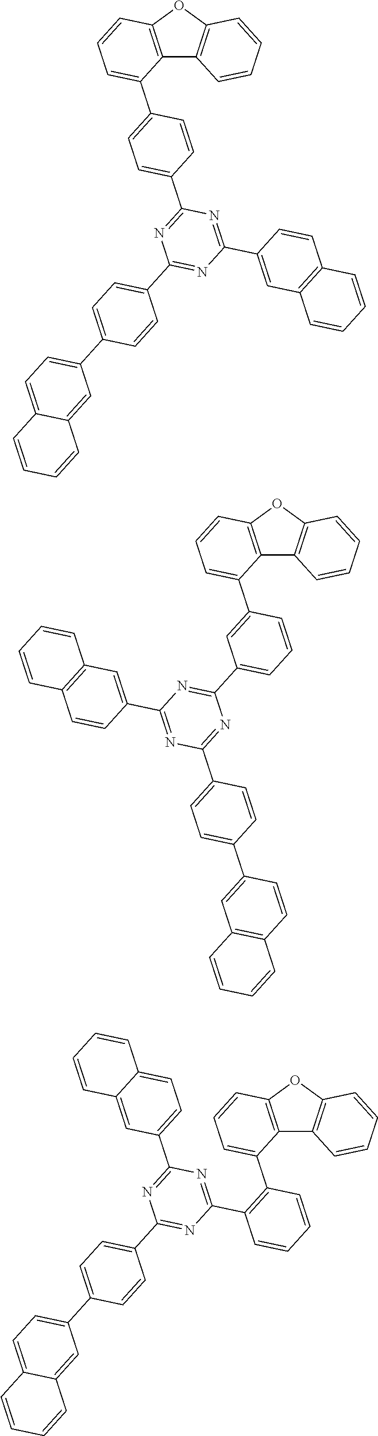 [SMILES: c1ccc2cc(-c3ccc(-c4nc(-c5ccc(-c6cccc7oc8ccccc8c67)cc5)nc(-c5ccc6ccccc6c5)n4)cc3)ccc2c1 ; c1cc(-c2nc(-c3ccc(-c4ccc5ccccc5c4)cc3)nc(-c3ccc4ccccc4c3)n2)cc(-c2cccc3oc4ccccc4c23)c1 ; c1ccc(-c2cccc3oc4ccccc4c23)c(-c2nc(-c3ccc(-c4ccc5ccccc5c4)cc3)nc(-c3ccc4ccccc4c3)n2)c1]

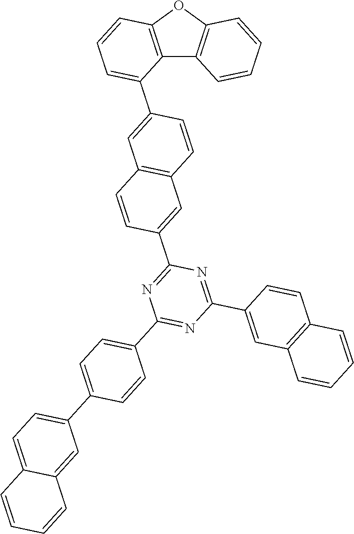 [SMILES: c1ccc2cc(-c3ccc(-c4nc(-c5ccc6ccccc6c5)nc(-c5ccc6cc(-c7cccc8oc9ccccc9c78)ccc6c5)n4)cc3)ccc2c1]

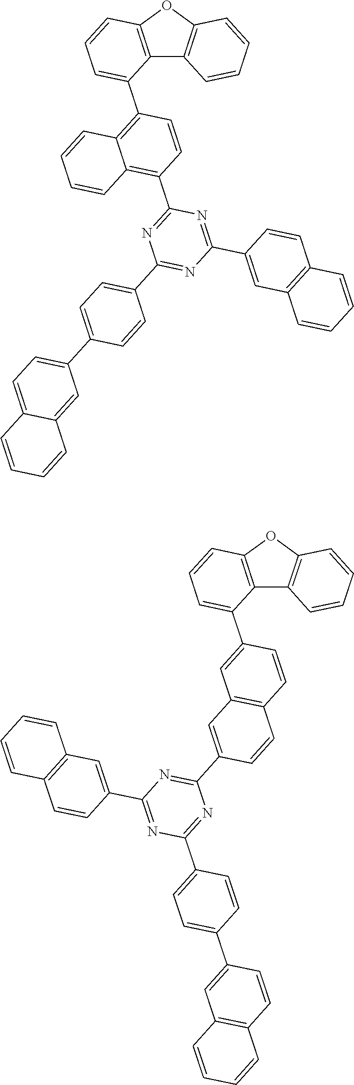 [SMILES: c1ccc2cc(-c3ccc(-c4nc(-c5ccc6ccccc6c5)nc(-c5ccc(-c6cccc7oc8ccccc8c67)c6ccccc56)n4)cc3)ccc2c1 ; c1ccc2cc(-c3ccc(-c4nc(-c5ccc6ccccc6c5)nc(-c5ccc6ccc(-c7cccc8oc9ccccc9c78)cc6c5)n4)cc3)ccc2c1]

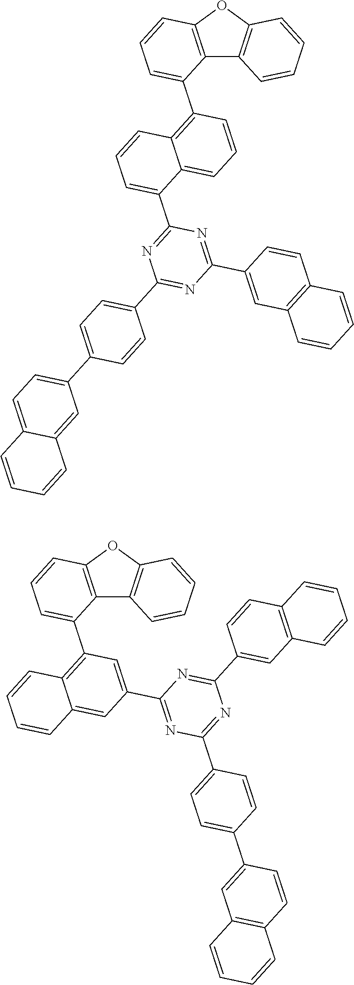 [SMILES: c1ccc2cc(-c3ccc(-c4nc(-c5ccc6ccccc6c5)nc(-c5cccc6c(-c7cccc8oc9ccccc9c78)cccc56)n4)cc3)ccc2c1 ; c1ccc2cc(-c3ccc(-c4nc(-c5ccc6ccccc6c5)nc(-c5cc(-c6cccc7oc8ccccc8c67)c6ccccc6c5)n4)cc3)ccc2c1]

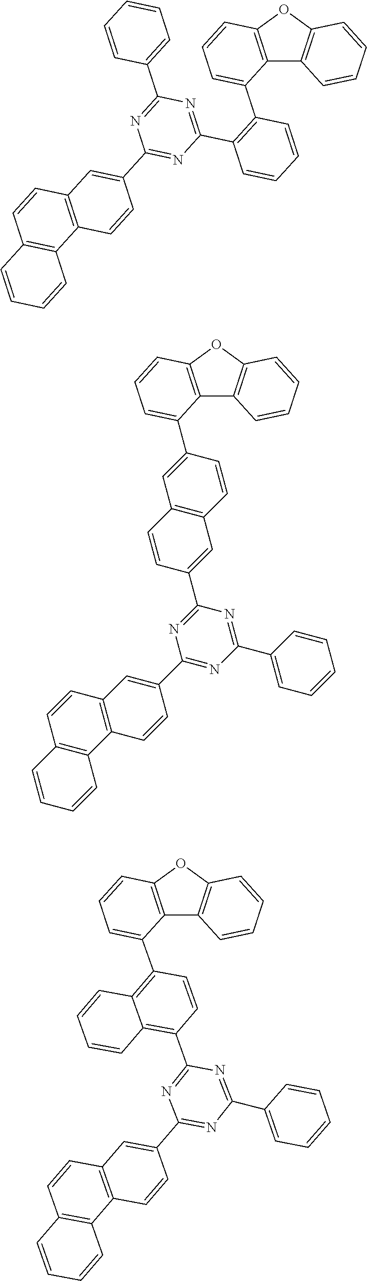 [SMILES: c1ccc(-c2nc(-c3ccc4c(ccc5ccccc54)c3)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4c(ccc5ccccc54)c3)n2)cc1 ; c1ccc(-c2nc(-c3ccc4c(ccc5ccccc54)c3)nc(-c3ccc(-c4cccc5oc6ccccc6c45)c4ccccc34)n2)cc1]

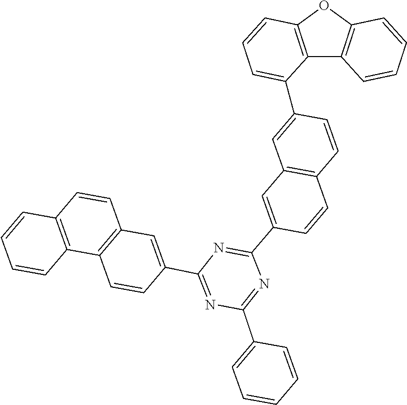 [SMILES: c1ccc(-c2nc(-c3ccc4ccc(-c5cccc6oc7ccccc7c56)cc4c3)nc(-c3ccc4c(ccc5ccccc54)c3)n2)cc1]

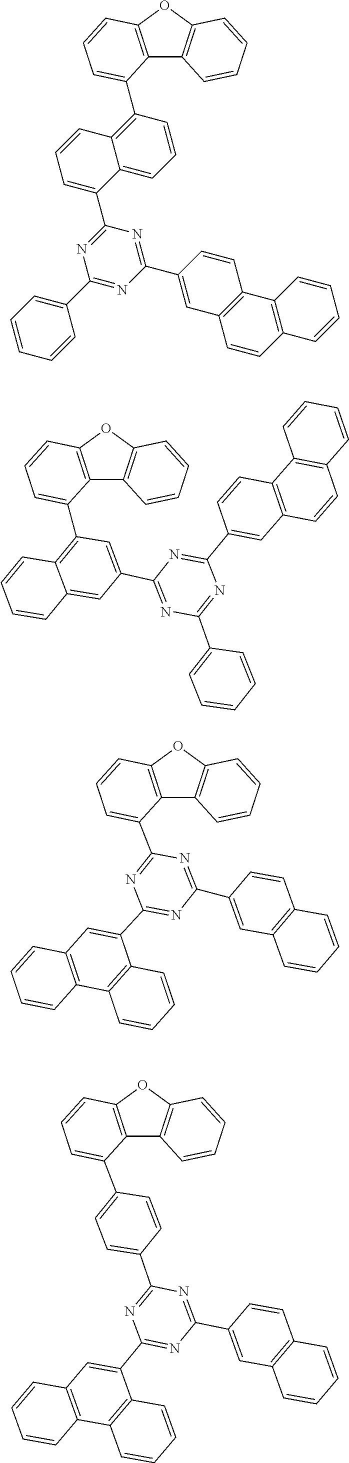 [SMILES: c1ccc(-c2nc(-c3ccc4c(ccc5ccccc54)c3)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4c(ccc5ccccc54)c3)n2)cc1 ; c1ccc2cc(-c3nc(-c4cc5ccccc5c5ccccc45)nc(-c4cccc5oc6ccccc6c45)n3)ccc2c1 ; c1ccc2cc(-c3nc(-c4ccc(-c5cccc6oc7ccccc7c56)cc4)nc(-c4cc5ccccc5c5ccccc45)n3)ccc2c1]

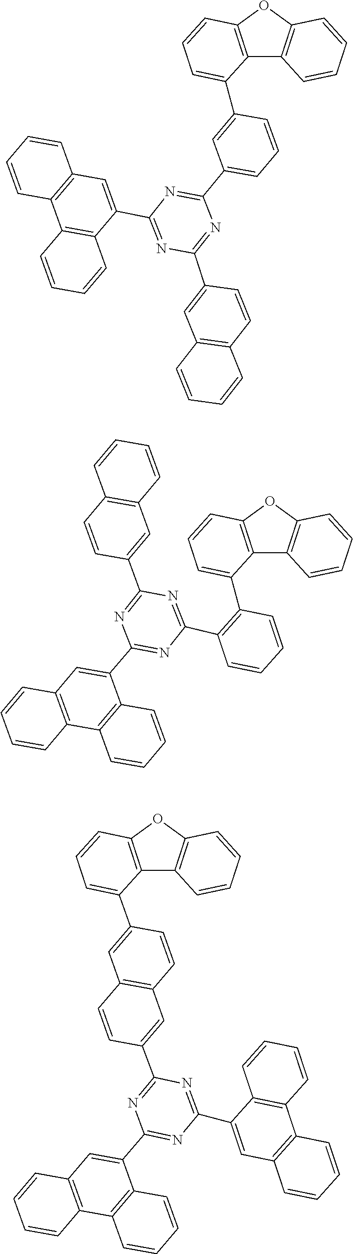 [SMILES: c1cc(-c2nc(-c3ccc4ccccc4c3)nc(-c3cc4ccccc4c4ccccc34)n2)cc(-c2cccc3oc4ccccc4c23)c1 ; c1ccc(-c2cccc3oc4ccccc4c23)c(-c2nc(-c3ccc4ccccc4c3)nc(-c3cc4ccccc4c4ccccc34)n2)c1 ; c1ccc2c(c1)cc(-c1nc(-c3ccc4cc(-c5cccc6oc7ccccc7c56)ccc4c3)nc(-c3cc4ccccc4c4ccccc34)n1)c1ccccc12]

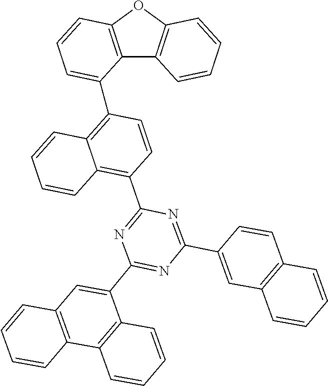 [SMILES: c1ccc2cc(-c3nc(-c4ccc(-c5cccc6oc7ccccc7c56)c5ccccc45)nc(-c4cc5ccccc5c5ccccc45)n3)ccc2c1]

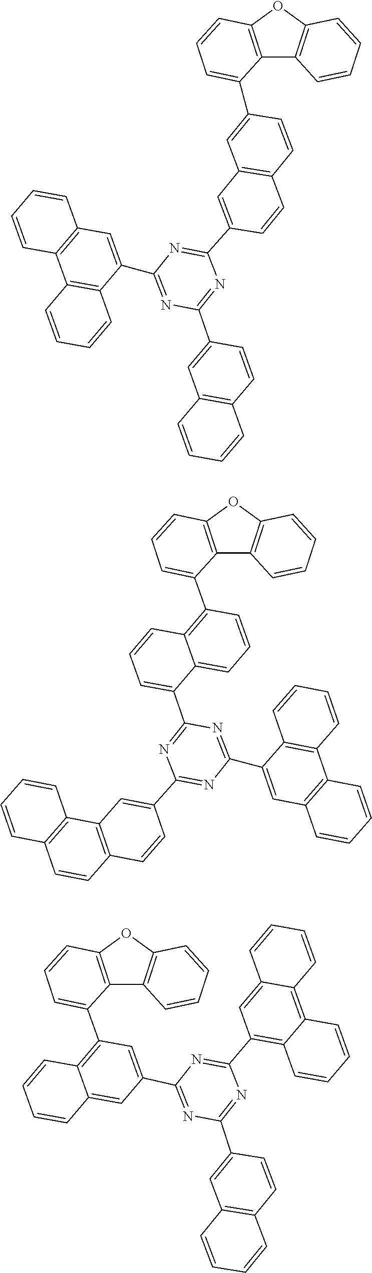

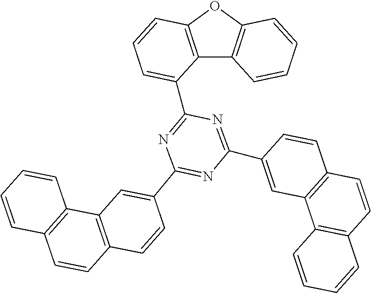 [SMILES: c1ccc2c(c1)ccc1ccc(-c3nc(-c4ccc5ccc6ccccc6c5c4)nc(-c4cccc5oc6ccccc6c45)n3)cc12]

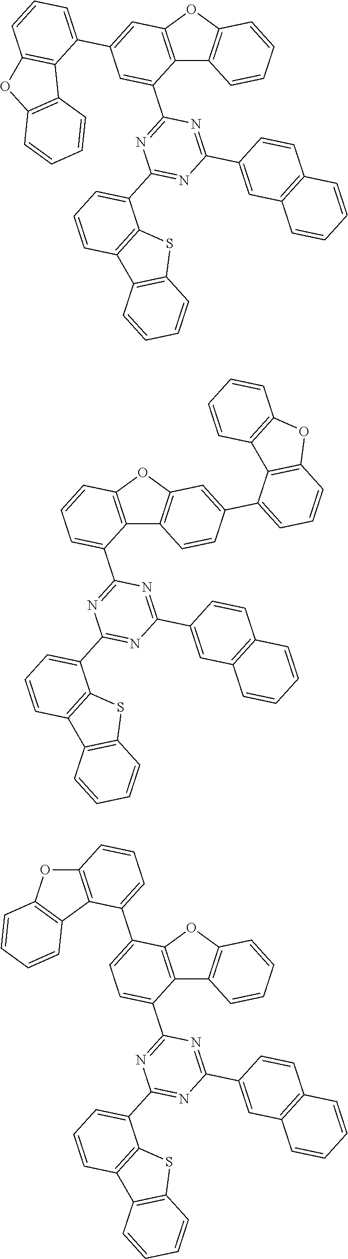

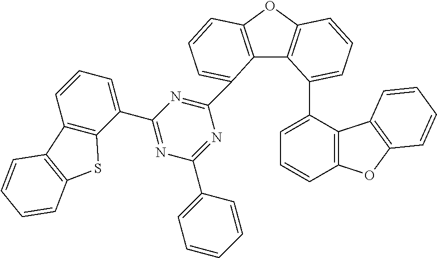 [SMILES: c1ccc(-c2nc(-c3cccc4c3sc3ccccc34)nc(-c3cccc4oc5cccc(-c6cccc7oc8ccccc8c67)c5c34)n2)cc1]

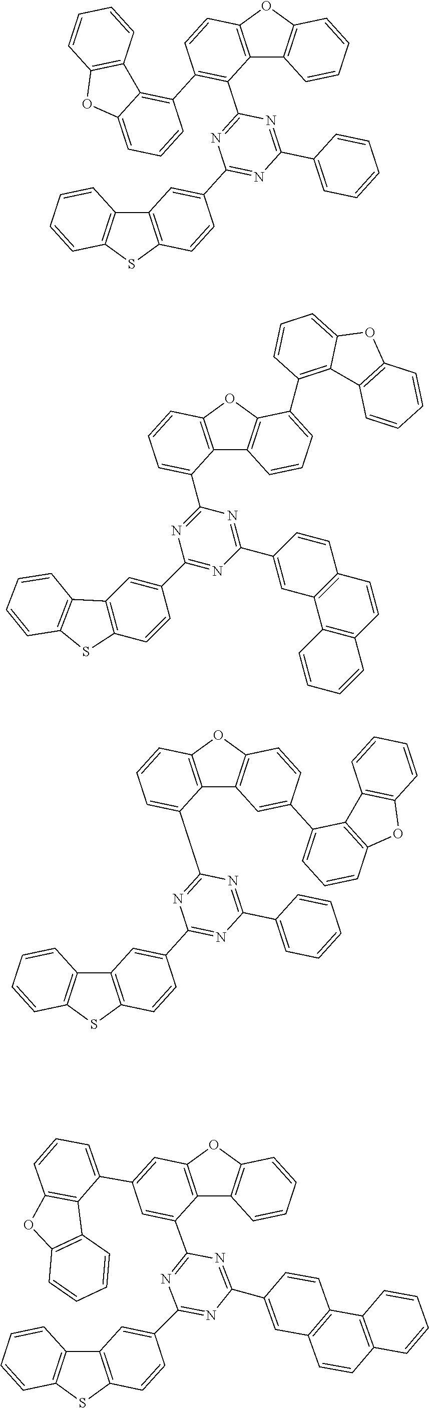 [SMILES: c1ccc(-c2nc(-c3ccc4sc5ccccc5c4c3)nc(-c3c(-c4cccc5oc6ccccc6c45)ccc4oc5ccccc5c34)n2)cc1 ; c1ccc2c(c1)ccc1ccc(-c3nc(-c4ccc5sc6ccccc6c5c4)nc(-c4cccc5oc6c(-c7cccc8oc9ccccc9c78)cccc6c45)n3)cc12 ; c1ccc(-c2nc(-c3ccc4sc5ccccc5c4c3)nc(-c3cccc4oc5ccc(-c6cccc7oc8ccccc8c67)cc5c34)n2)cc1 ; c1ccc2c(c1)ccc1cc(-c3nc(-c4ccc5sc6ccccc6c5c4)nc(-c4cc(-c5cccc6oc7ccccc7c56)cc5oc6ccccc6c45)n3)ccc12]

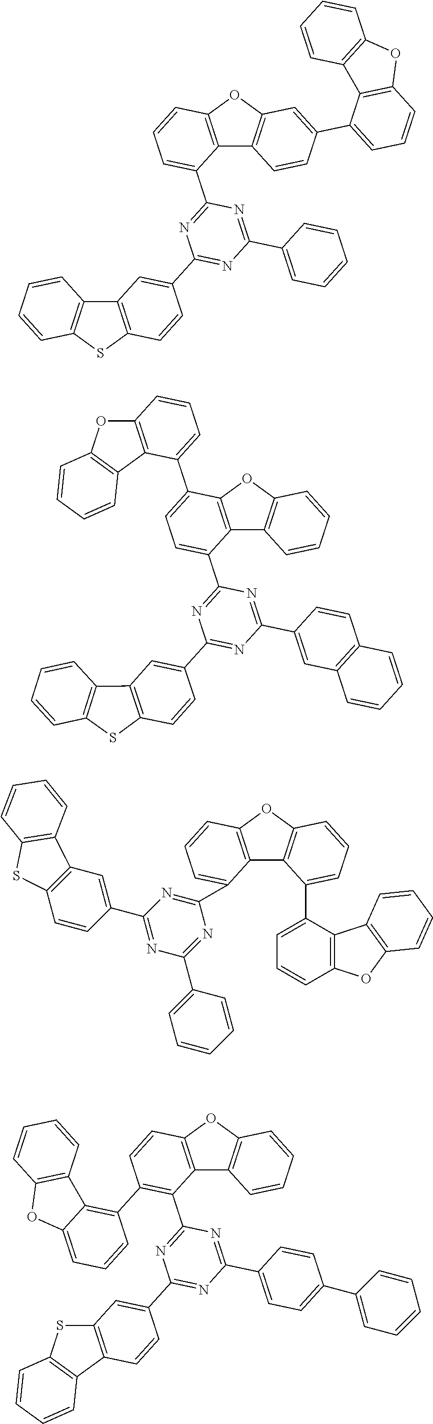 [SMILES: c1ccc(-c2nc(-c3ccc4sc5ccccc5c4c3)nc(-c3cccc4oc5cc(-c6cccc7oc8ccccc8c67)ccc5c34)n2)cc1 ; c1ccc2cc(-c3nc(-c4ccc5sc6ccccc6c5c4)nc(-c4ccc(-c5cccc6oc7ccccc7c56)c5oc6ccccc6c45)n3)ccc2c1 ; c1ccc(-c2nc(-c3ccc4sc5ccccc5c4c3)nc(-c3cccc4oc5cccc(-c6cccc7oc8ccccc8c67)c5c34)n2)cc1 ; c1ccc(-c2ccc(-c3nc(-c4ccc5c(c4)sc4ccccc45)nc(-c4c(-c5cccc6oc7ccccc7c56)ccc5oc6ccccc6c45)n3)cc2)cc1]

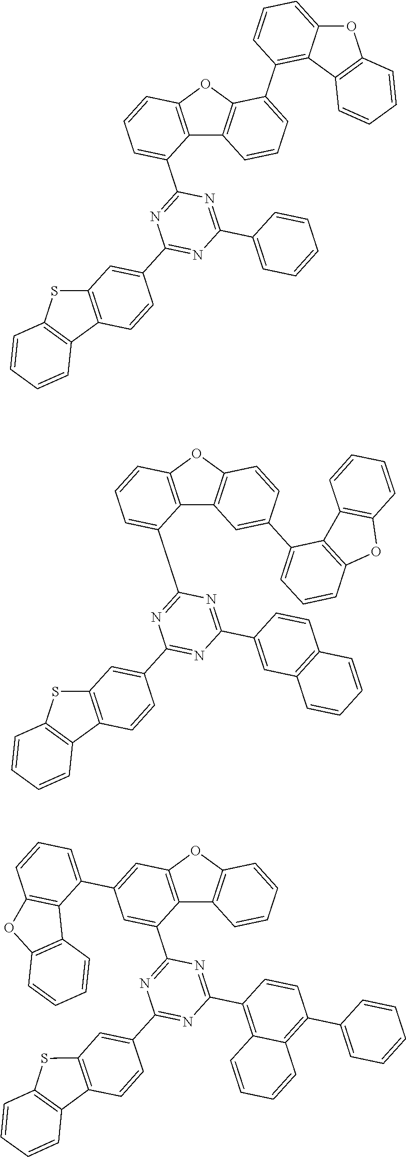 [SMILES: c1ccc(-c2nc(-c3ccc4c(c3)sc3ccccc34)nc(-c3cccc4oc5c(-c6cccc7oc8ccccc8c67)cccc5c34)n2)cc1 ; c1ccc2cc(-c3nc(-c4ccc5c(c4)sc4ccccc45)nc(-c4cccc5oc6ccc(-c7cccc8oc9ccccc9c78)cc6c45)n3)ccc2c1 ; c1ccc(-c2ccc(-c3nc(-c4ccc5c(c4)sc4ccccc45)nc(-c4cc(-c5cccc6oc7ccccc7c56)cc5oc6ccccc6c45)n3)c3ccccc23)cc1]

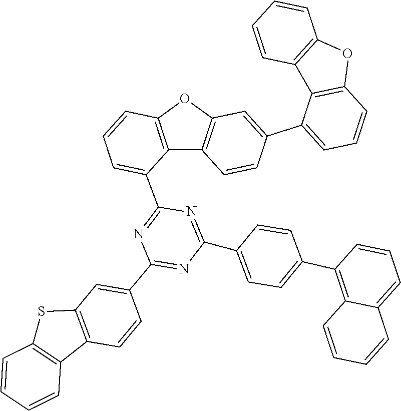 [SMILES: c1ccc2c(-c3ccc(-c4nc(-c5ccc6c(c5)sc5ccccc56)nc(-c5cccc6oc7cc(-c8cccc9oc%10ccccc%10c89)ccc7c56)n4)cc3)cccc2c1]

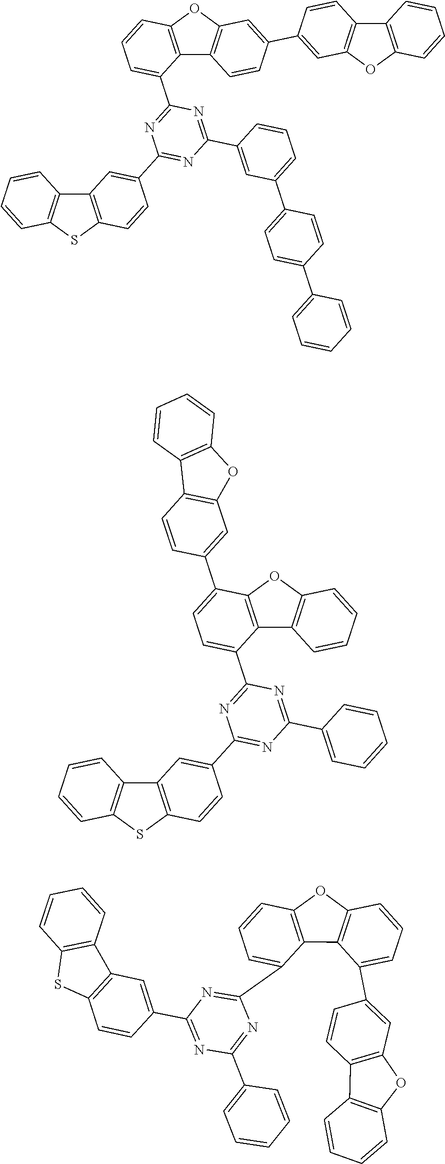 [SMILES: c1ccc(-c2ccc(-c3cccc(-c4nc(-c5ccc6sc7ccccc7c6c5)nc(-c5cccc6oc7cc(-c8ccc9c(c8)oc8ccccc89)ccc7c56)n4)c3)cc2)cc1 ; c1ccc(-c2nc(-c3ccc4sc5ccccc5c4c3)nc(-c3ccc(-c4ccc5c(c4)oc4ccccc45)c4oc5ccccc5c34)n2)cc1 ; c1ccc(-c2nc(-c3ccc4sc5ccccc5c4c3)nc(-c3cccc4oc5cccc(-c6ccc7c(c6)oc6ccccc67)c5c34)n2)cc1]

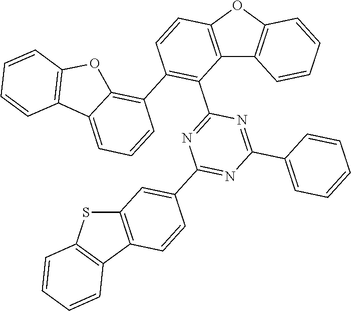 [SMILES: c1ccc(-c2nc(-c3ccc4c(c3)sc3ccccc34)nc(-c3c(-c4cccc5c4oc4ccccc45)ccc4oc5ccccc5c34)n2)cc1]

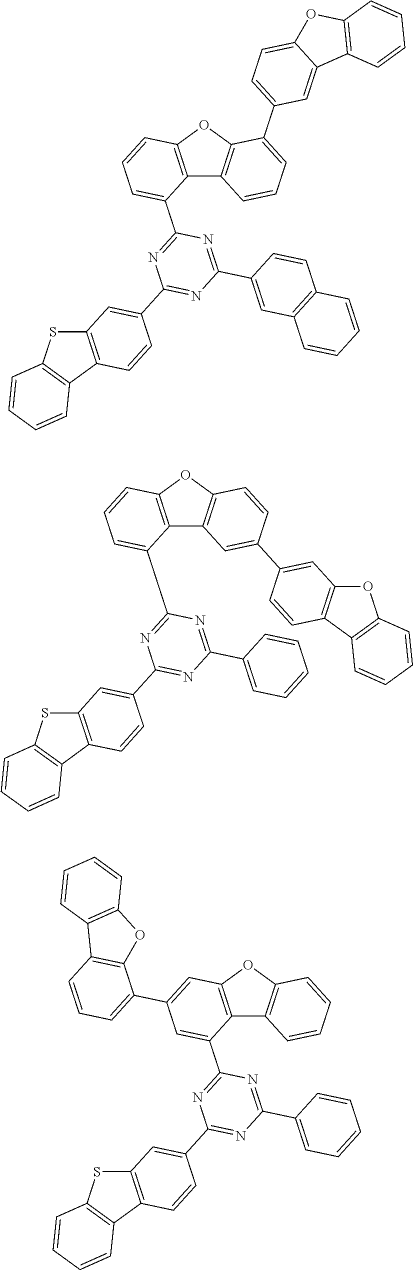 [SMILES: c1ccc2cc(-c3nc(-c4ccc5c(c4)sc4ccccc45)nc(-c4cccc5oc6c(-c7ccc8oc9ccccc9c8c7)cccc6c45)n3)ccc2c1 ; c1ccc(-c2nc(-c3ccc4c(c3)sc3ccccc34)nc(-c3cccc4oc5ccc(-c6ccc7c(c6)oc6ccccc67)cc5c34)n2)cc1 ; c1ccc(-c2nc(-c3ccc4c(c3)sc3ccccc34)nc(-c3cc(-c4cccc5c4oc4ccccc45)cc4oc5ccccc5c34)n2)cc1]

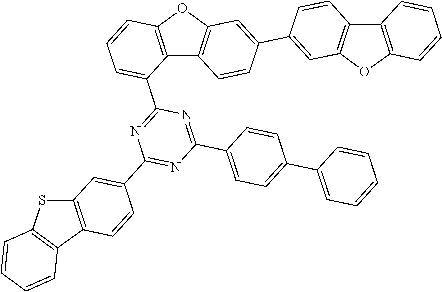 [SMILES: c1ccc(-c2ccc(-c3nc(-c4ccc5c(c4)sc4ccccc45)nc(-c4cccc5oc6cc(-c7ccc8c(c7)oc7ccccc78)ccc6c45)n3)cc2)cc1]

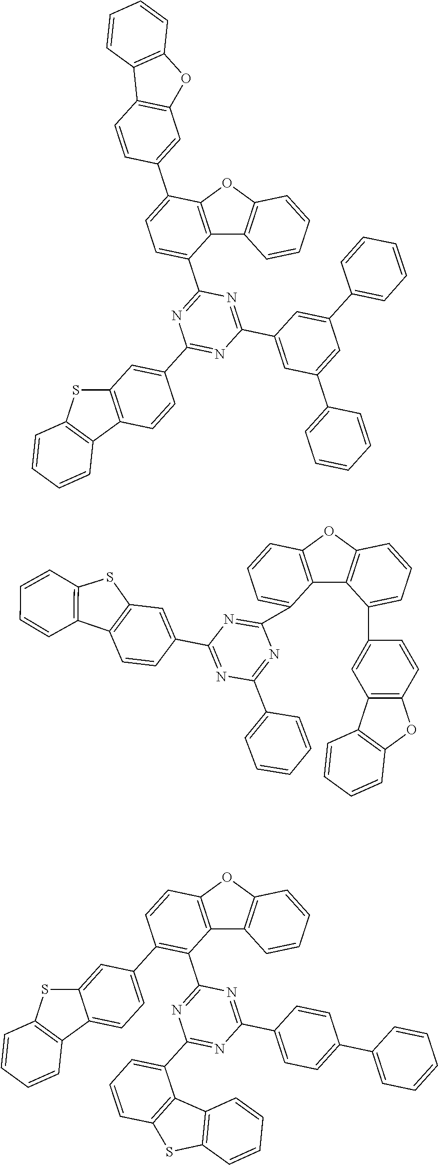 [SMILES: c1ccc(-c2cc(-c3ccccc3)cc(-c3nc(-c4ccc5c(c4)sc4ccccc45)nc(-c4ccc(-c5ccc6c(c5)oc5ccccc56)c5oc6ccccc6c45)n3)c2)cc1 ; c1ccc(-c2nc(-c3ccc4c(c3)sc3ccccc34)nc(-c3cccc4oc5cccc(-c6ccc7oc8ccccc8c7c6)c5c34)n2)cc1 ; c1ccc(-c2ccc(-c3nc(-c4c(-c5ccc6c(c5)sc5ccccc56)ccc5oc6ccccc6c45)nc(-c4cccc5sc6ccccc6c45)n3)cc2)cc1]

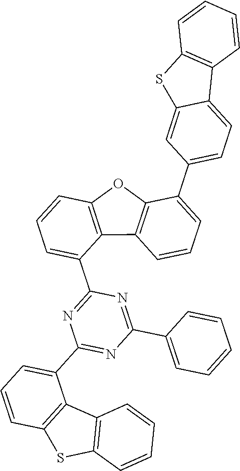 [SMILES: c1ccc(-c2nc(-c3cccc4oc5c(-c6ccc7c(c6)sc6ccccc67)cccc5c34)nc(-c3cccc4sc5ccccc5c34)n2)cc1]

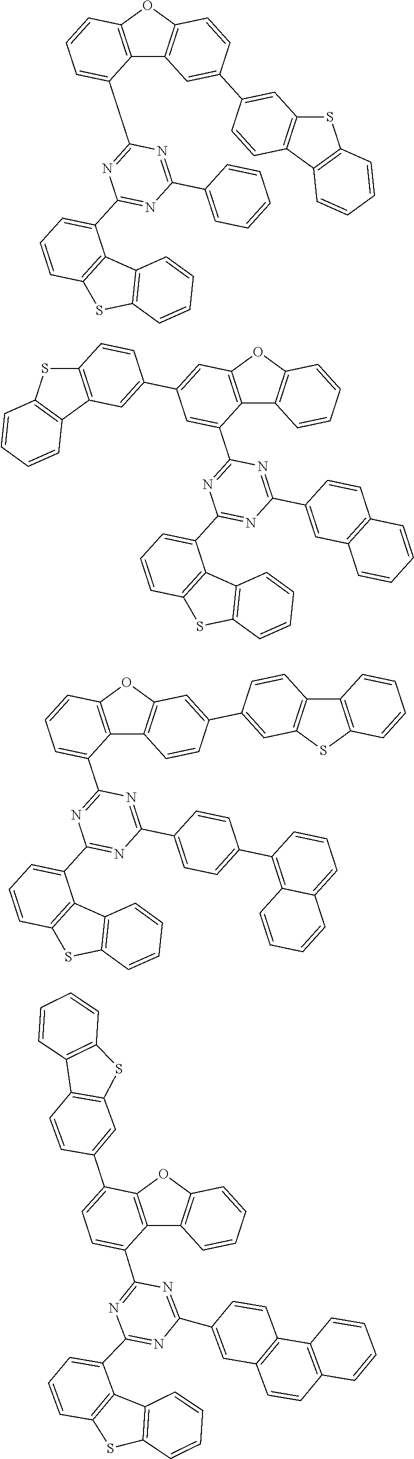

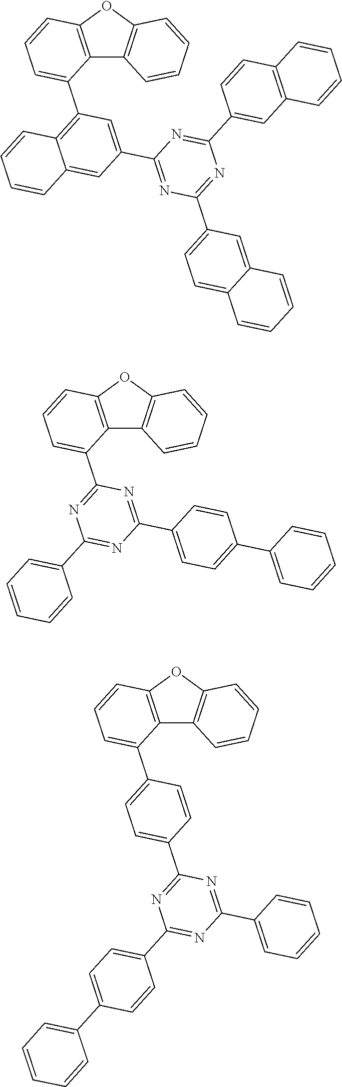 [SMILES: c1ccc2cc(-c3nc(-c4ccc5ccccc5c4)nc(-c4cc(-c5cccc6oc7ccccc7c56)c5ccccc5c4)n3)ccc2c1 ; c1ccc(-c2ccc(-c3nc(-c4ccccc4)nc(-c4cccc5oc6ccccc6c45)n3)cc2)cc1 ; c1ccc(-c2ccc(-c3nc(-c4ccccc4)nc(-c4ccc(-c5cccc6oc7ccccc7c56)cc4)n3)cc2)cc1]

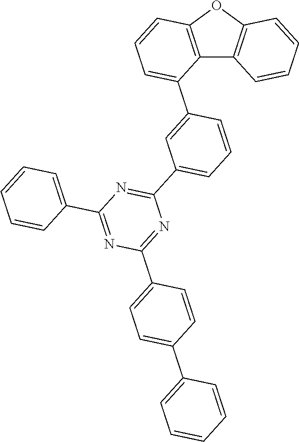 [SMILES: c1ccc(-c2ccc(-c3nc(-c4ccccc4)nc(-c4cccc(-c5cccc6oc7ccccc7c56)c4)n3)cc2)cc1]

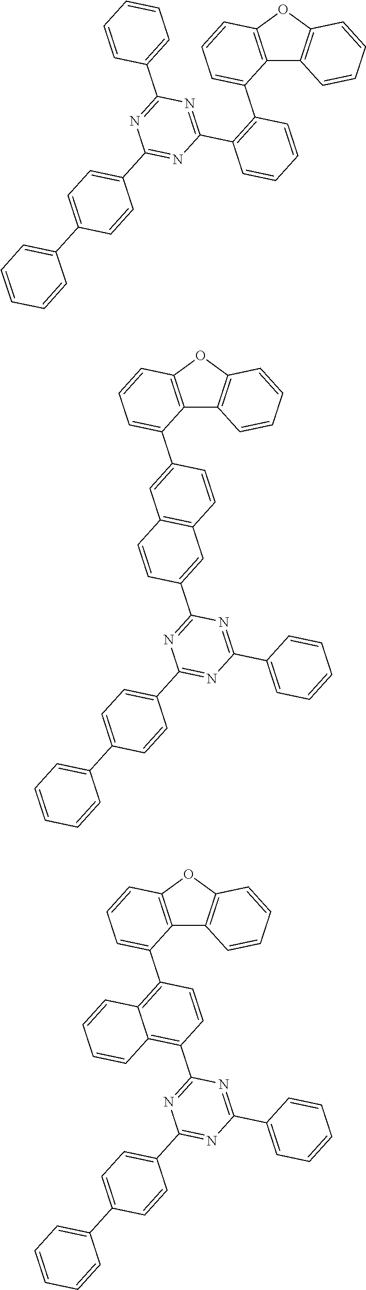 [SMILES: c1ccc(-c2ccc(-c3nc(-c4ccccc4)nc(-c4ccccc4-c4cccc5oc6ccccc6c45)n3)cc2)cc1 ; c1ccc(-c2ccc(-c3nc(-c4ccccc4)nc(-c4ccc5cc(-c6cccc7oc8ccccc8c67)ccc5c4)n3)cc2)cc1 ; c1ccc(-c2ccc(-c3nc(-c4ccccc4)nc(-c4ccc(-c5cccc6oc7ccccc7c56)c5ccccc45)n3)cc2)cc1]

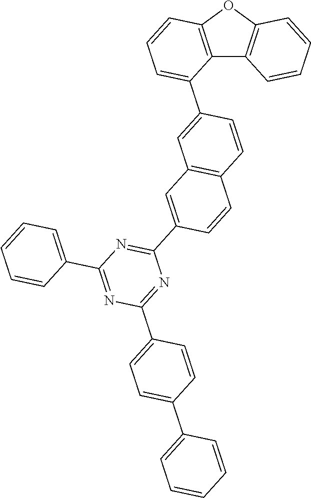 [SMILES: c1ccc(-c2ccc(-c3nc(-c4ccccc4)nc(-c4ccc5ccc(-c6cccc7oc8ccccc8c67)cc5c4)n3)cc2)cc1]

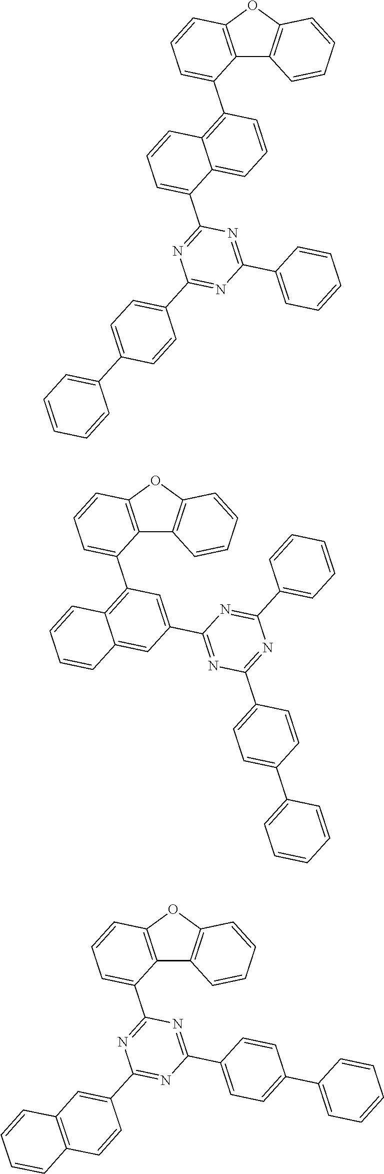 [SMILES: c1ccc(-c2ccc(-c3nc(-c4ccccc4)nc(-c4cccc5c(-c6cccc7oc8ccccc8c67)cccc45)n3)cc2)cc1 ; c1ccc(-c2ccc(-c3nc(-c4ccccc4)nc(-c4cc(-c5cccc6oc7ccccc7c56)c5ccccc5c4)n3)cc2)cc1 ; c1ccc(-c2ccc(-c3nc(-c4ccc5ccccc5c4)nc(-c4cccc5oc6ccccc6c45)n3)cc2)cc1]

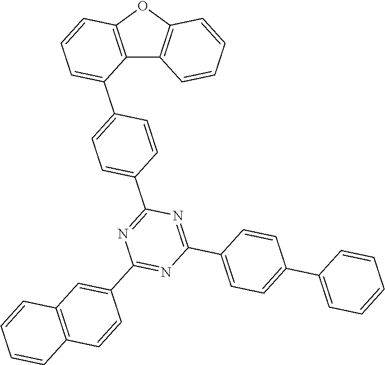 [SMILES: c1ccc(-c2ccc(-c3nc(-c4ccc(-c5cccc6oc7ccccc7c56)cc4)nc(-c4ccc5ccccc5c4)n3)cc2)cc1]

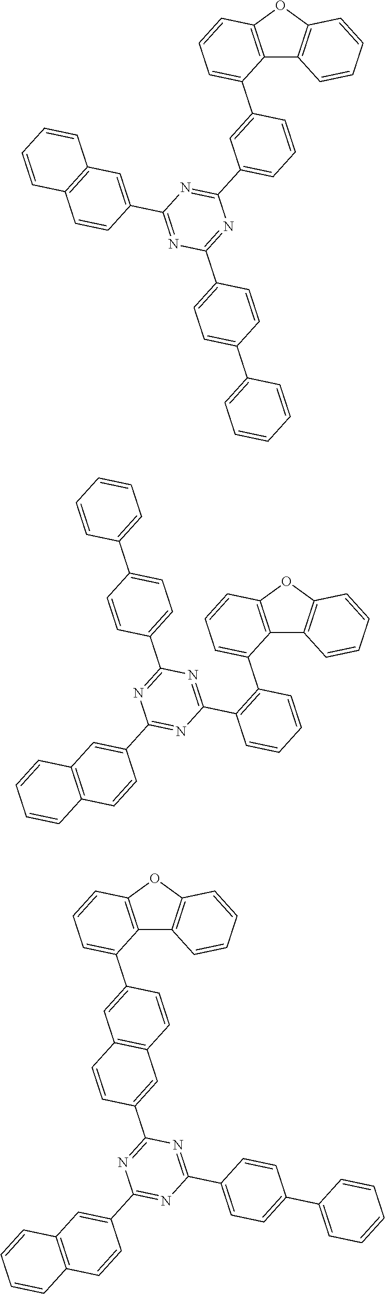 [SMILES: c1ccc(-c2ccc(-c3nc(-c4cccc(-c5cccc6oc7ccccc7c56)c4)nc(-c4ccc5ccccc5c4)n3)cc2)cc1 ; c1ccc(-c2ccc(-c3nc(-c4ccc5ccccc5c4)nc(-c4ccccc4-c4cccc5oc6ccccc6c45)n3)cc2)cc1 ; c1ccc(-c2ccc(-c3nc(-c4ccc5ccccc5c4)nc(-c4ccc5cc(-c6cccc7oc8ccccc8c67)ccc5c4)n3)cc2)cc1]

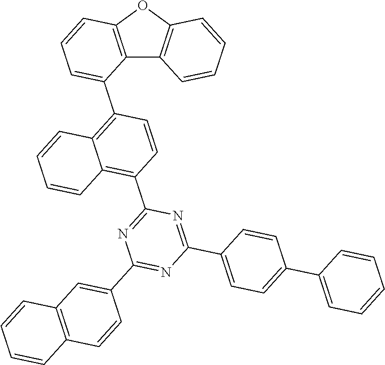 [SMILES: c1ccc(-c2ccc(-c3nc(-c4ccc5ccccc5c4)nc(-c4ccc(-c5cccc6oc7ccccc7c56)c5ccccc45)n3)cc2)cc1]

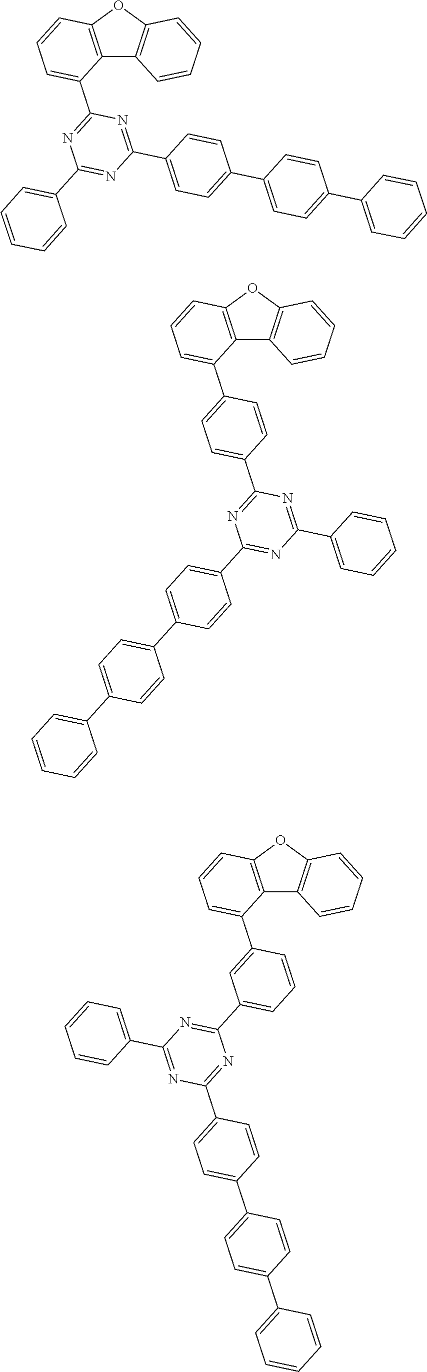 [SMILES: c1ccc(-c2ccc(-c3ccc(-c4nc(-c5ccccc5)nc(-c5cccc6oc7ccccc7c56)n4)cc3)cc2)cc1 ; c1ccc(-c2ccc(-c3ccc(-c4nc(-c5ccccc5)nc(-c5ccc(-c6cccc7oc8ccccc8c67)cc5)n4)cc3)cc2)cc1 ; c1ccc(-c2ccc(-c3ccc(-c4nc(-c5ccccc5)nc(-c5cccc(-c6cccc7oc8ccccc8c67)c5)n4)cc3)cc2)cc1]

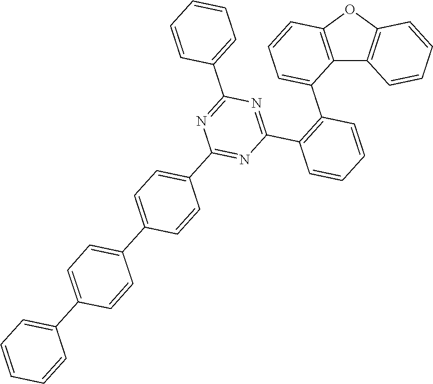 [SMILES: c1ccc(-c2ccc(-c3ccc(-c4nc(-c5ccccc5)nc(-c5ccccc5-c5cccc6oc7ccccc7c56)n4)cc3)cc2)cc1]

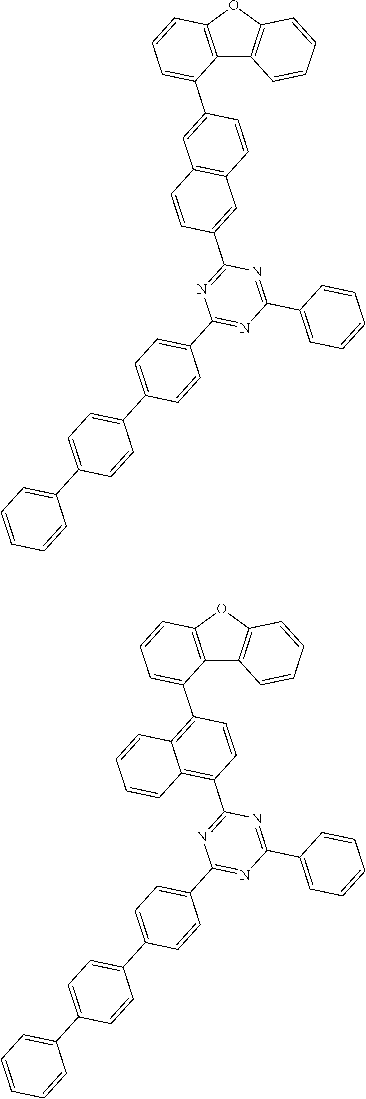 [SMILES: c1ccc(-c2ccc(-c3ccc(-c4nc(-c5ccccc5)nc(-c5ccc6cc(-c7cccc8oc9ccccc9c78)ccc6c5)n4)cc3)cc2)cc1 ; c1ccc(-c2ccc(-c3ccc(-c4nc(-c5ccccc5)nc(-c5ccc(-c6cccc7oc8ccccc8c67)c6ccccc56)n4)cc3)cc2)cc1]

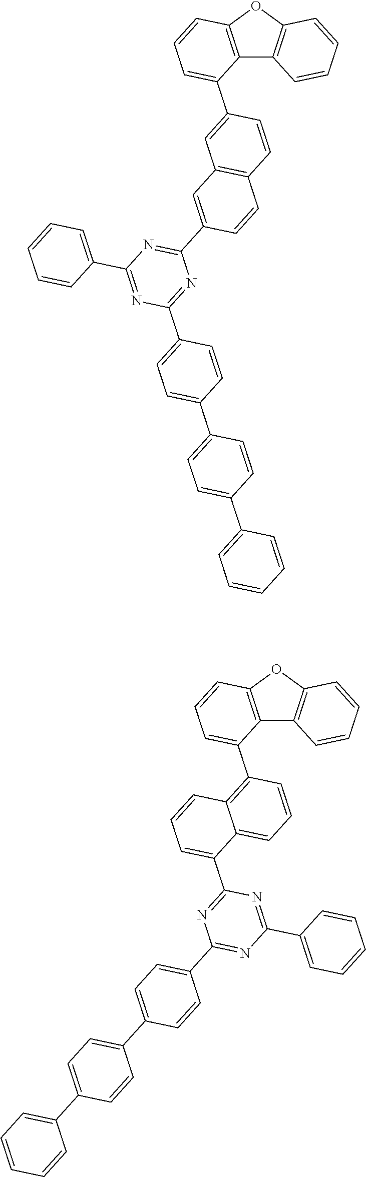 [SMILES: c1ccc(-c2ccc(-c3ccc(-c4nc(-c5ccccc5)nc(-c5ccc6ccc(-c7cccc8oc9ccccc9c78)cc6c5)n4)cc3)cc2)cc1 ; c1ccc(-c2ccc(-c3ccc(-c4nc(-c5ccccc5)nc(-c5cccc6c(-c7cccc8oc9ccccc9c78)cccc56)n4)cc3)cc2)cc1]

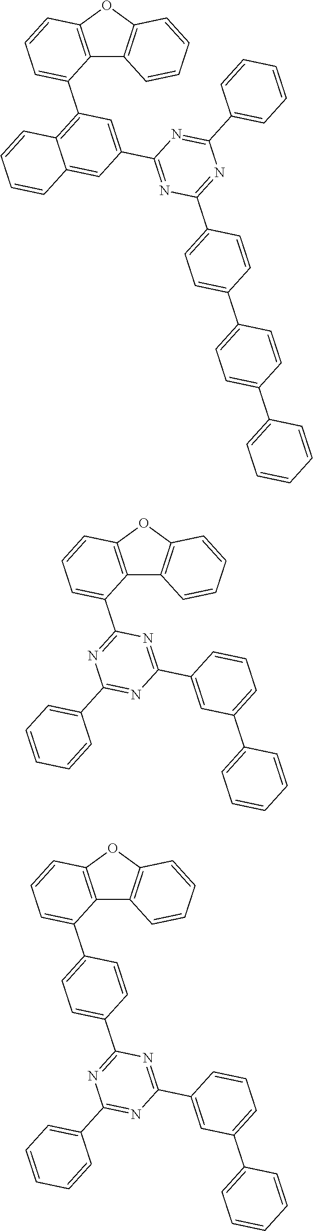 [SMILES: c1ccc(-c2ccc(-c3ccc(-c4nc(-c5ccccc5)nc(-c5cc(-c6cccc7oc8ccccc8c67)c6ccccc6c5)n4)cc3)cc2)cc1 ; c1ccc(-c2cccc(-c3nc(-c4ccccc4)nc(-c4cccc5oc6ccccc6c45)n3)c2)cc1 ; c1ccc(-c2cccc(-c3nc(-c4ccccc4)nc(-c4ccc(-c5cccc6oc7ccccc7c56)cc4)n3)c2)cc1]

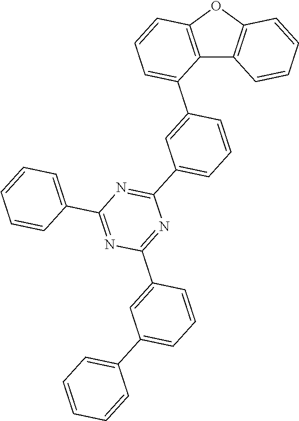 [SMILES: c1ccc(-c2cccc(-c3nc(-c4ccccc4)nc(-c4cccc(-c5cccc6oc7ccccc7c56)c4)n3)c2)cc1]

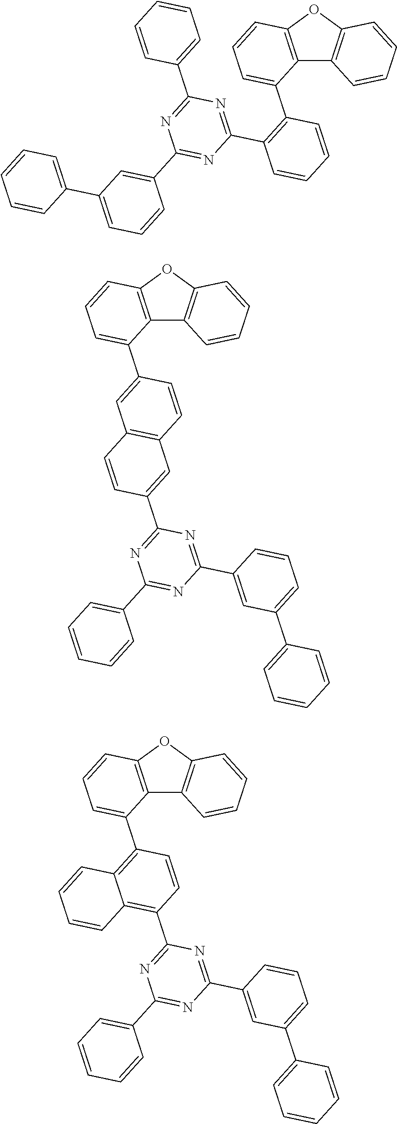

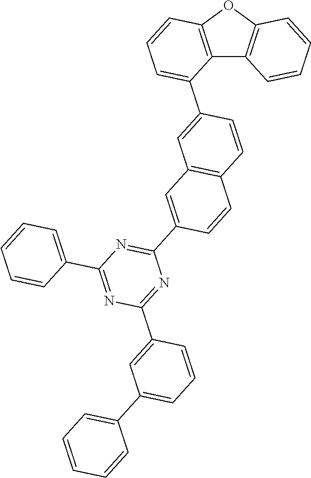 [SMILES: c1ccc(-c2cccc(-c3nc(-c4ccccc4)nc(-c4ccc5ccc(-c6cccc7oc8ccccc8c67)cc5c4)n3)c2)cc1]

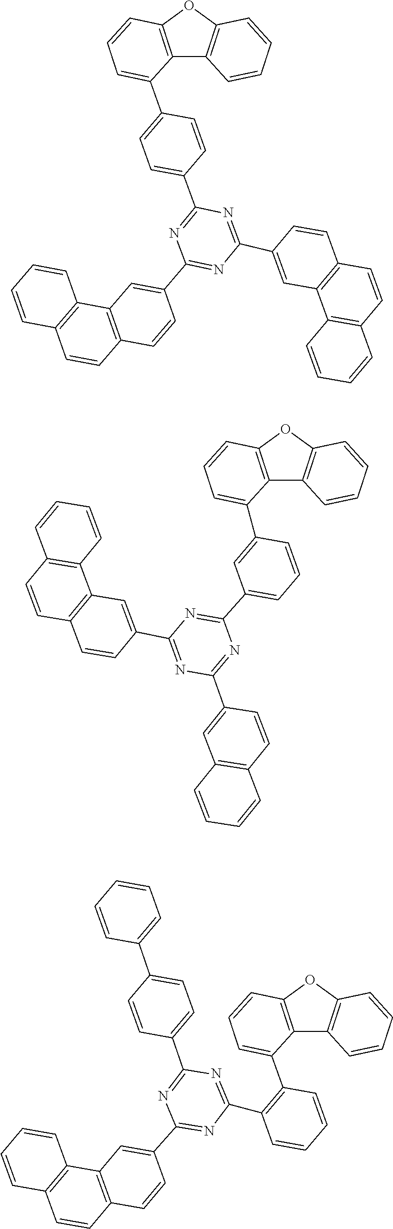 [SMILES: c1ccc2c(c1)ccc1ccc(-c3nc(-c4ccc(-c5cccc6oc7ccccc7c56)cc4)nc(-c4ccc5ccc6ccccc6c5c4)n3)cc12 ; c1cc(-c2nc(-c3ccc4ccccc4c3)nc(-c3ccc4ccc5ccccc5c4c3)n2)cc(-c2cccc3oc4ccccc4c23)c1 ; c1ccc(-c2ccc(-c3nc(-c4ccc5ccc6ccccc6c5c4)nc(-c4ccccc4-c4cccc5oc6ccccc6c45)n3)cc2)cc1]

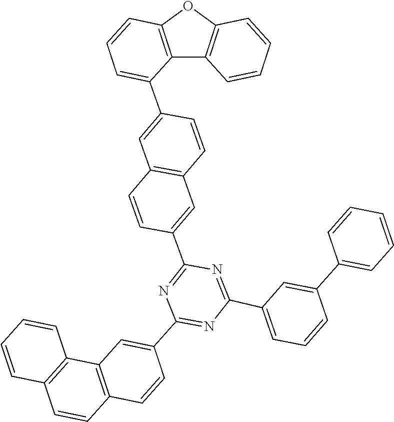 [SMILES: c1ccc(-c2cccc(-c3nc(-c4ccc5cc(-c6cccc7oc8ccccc8c67)ccc5c4)nc(-c4ccc5ccc6ccccc6c5c4)n3)c2)cc1]

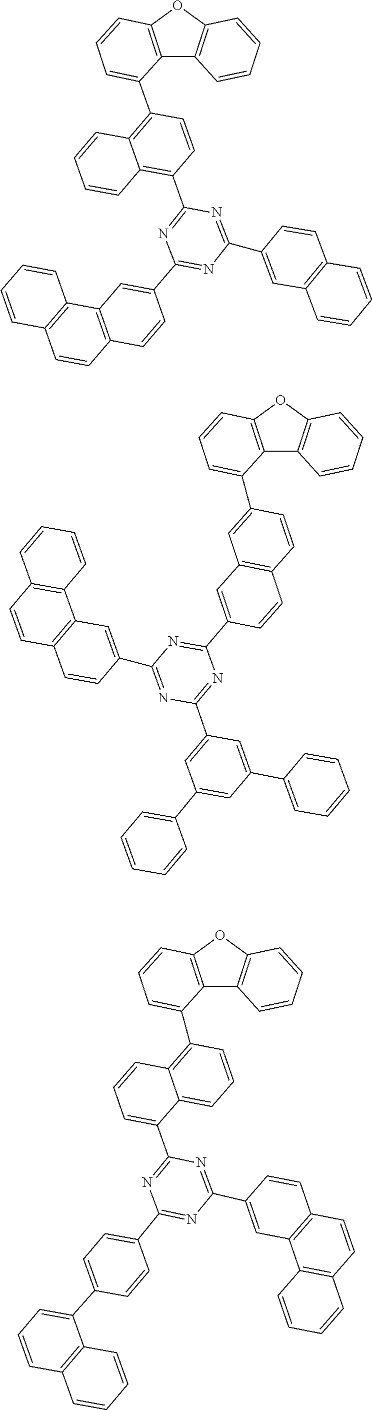 [SMILES: c1ccc2cc(-c3nc(-c4ccc5ccc6ccccc6c5c4)nc(-c4ccc(-c5cccc6oc7ccccc7c56)c5ccccc45)n3)ccc2c1 ; c1ccc(-c2cc(-c3ccccc3)cc(-c3nc(-c4ccc5ccc(-c6cccc7oc8ccccc8c67)cc5c4)nc(-c4ccc5ccc6ccccc6c5c4)n3)c2)cc1 ; c1ccc2c(-c3ccc(-c4nc(-c5ccc6ccc7ccccc7c6c5)nc(-c5cccc6c(-c7cccc8oc9ccccc9c78)cccc56)n4)cc3)cccc2c1]

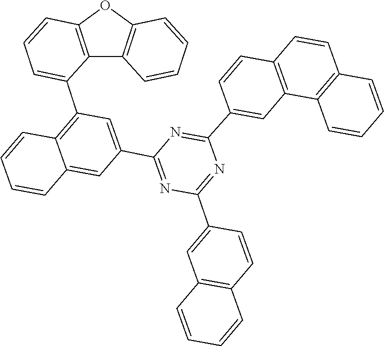 [SMILES: c1ccc2cc(-c3nc(-c4cc(-c5cccc6oc7ccccc7c56)c5ccccc5c4)nc(-c4ccc5ccc6ccccc6c5c4)n3)ccc2c1]

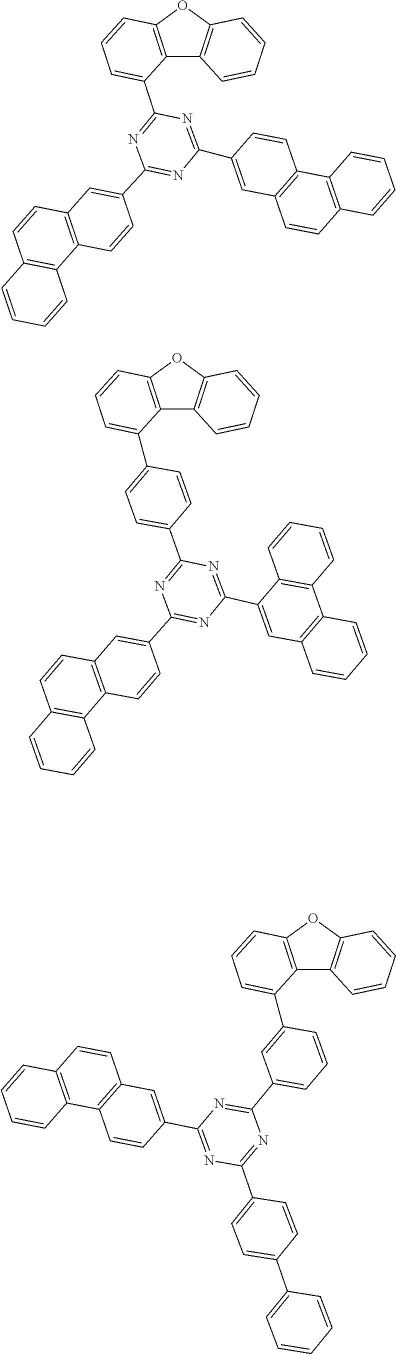 [SMILES: c1ccc2c(c1)ccc1cc(-c3nc(-c4ccc5c(ccc6ccccc65)c4)nc(-c4cccc5oc6ccccc6c45)n3)ccc12 ; c1ccc2c(c1)ccc1cc(-c3nc(-c4ccc(-c5cccc6oc7ccccc7c56)cc4)nc(-c4cc5ccccc5c5ccccc45)n3)ccc12 ; c1ccc(-c2ccc(-c3nc(-c4cccc(-c5cccc6oc7ccccc7c56)c4)nc(-c4ccc5c(ccc6ccccc65)c4)n3)cc2)cc1]

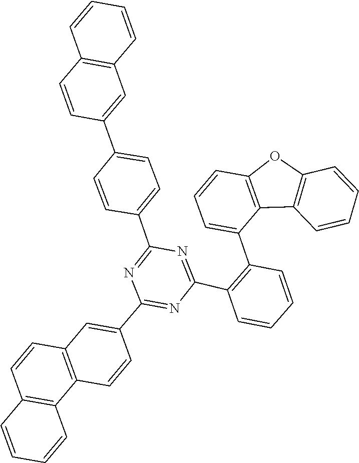 [SMILES: c1ccc(-c2cccc3oc4ccccc4c23)c(-c2nc(-c3ccc(-c4ccc5ccccc5c4)cc3)nc(-c3ccc4c(ccc5ccccc54)c3)n2)c1]

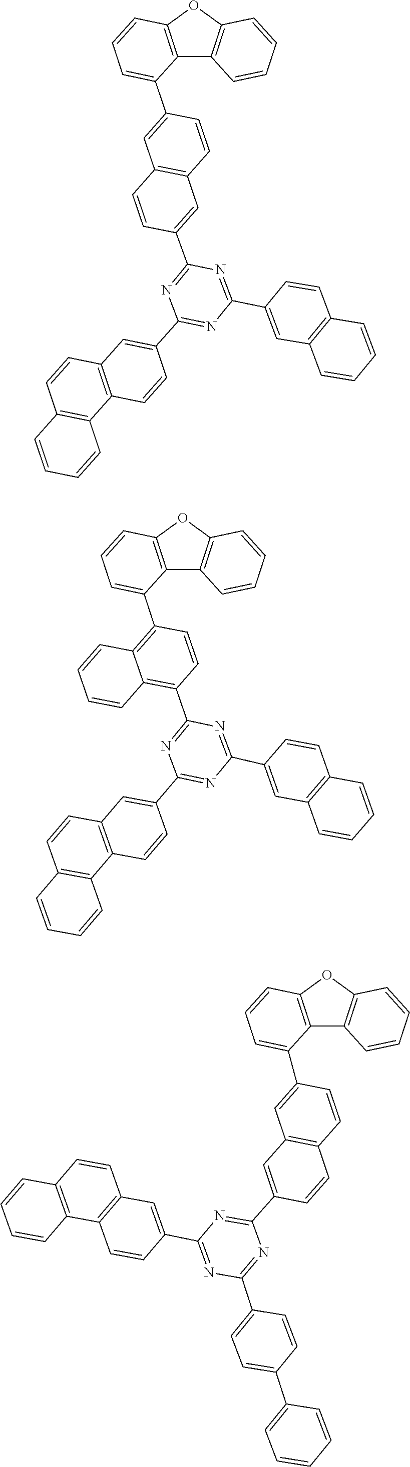 [SMILES: c1ccc2cc(-c3nc(-c4ccc5cc(-c6cccc7oc8ccccc8c67)ccc5c4)nc(-c4ccc5c(ccc6ccccc65)c4)n3)ccc2c1 ; c1ccc2cc(-c3nc(-c4ccc5c(ccc6ccccc65)c4)nc(-c4ccc(-c5cccc6oc7ccccc7c56)c5ccccc45)n3)ccc2c1 ; c1ccc(-c2ccc(-c3nc(-c4ccc5ccc(-c6cccc7oc8ccccc8c67)cc5c4)nc(-c4ccc5c(ccc6ccccc65)c4)n3)cc2)cc1]

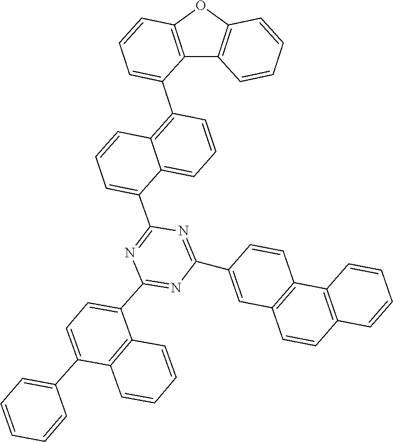 [SMILES: c1ccc(-c2ccc(-c3nc(-c4ccc5c(ccc6ccccc65)c4)nc(-c4cccc5c(-c6cccc7oc8ccccc8c67)cccc45)n3)c3ccccc23)cc1]

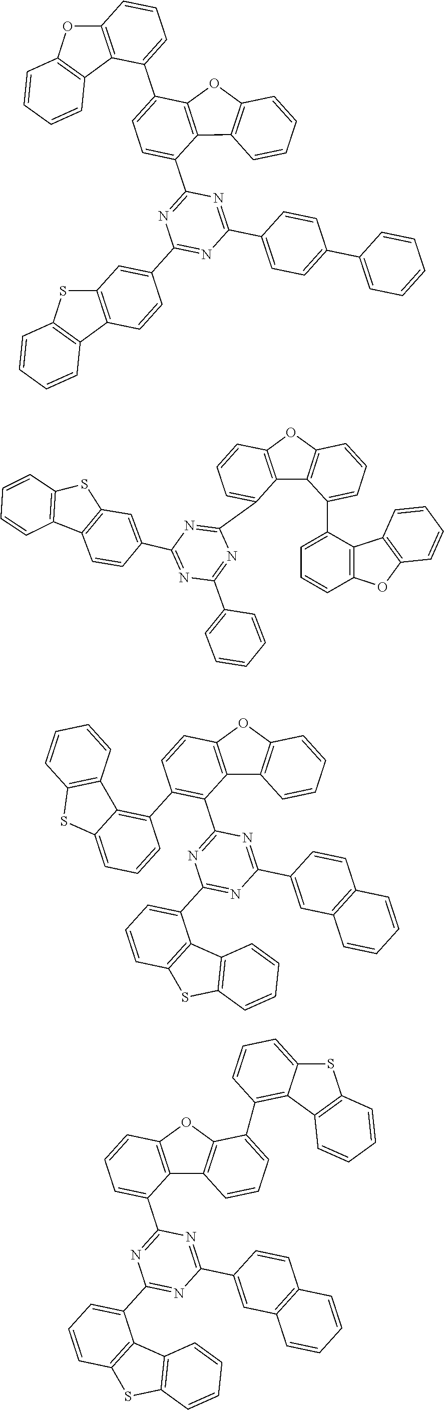

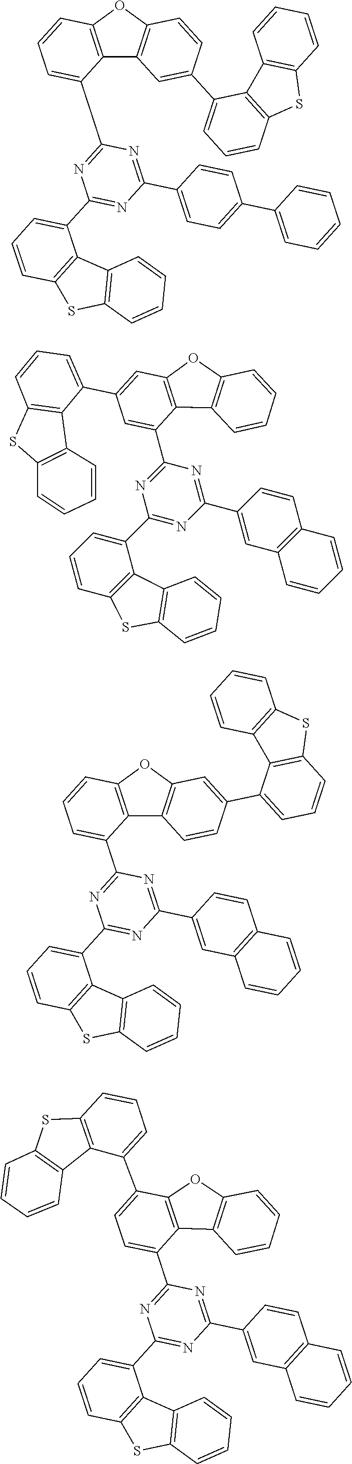

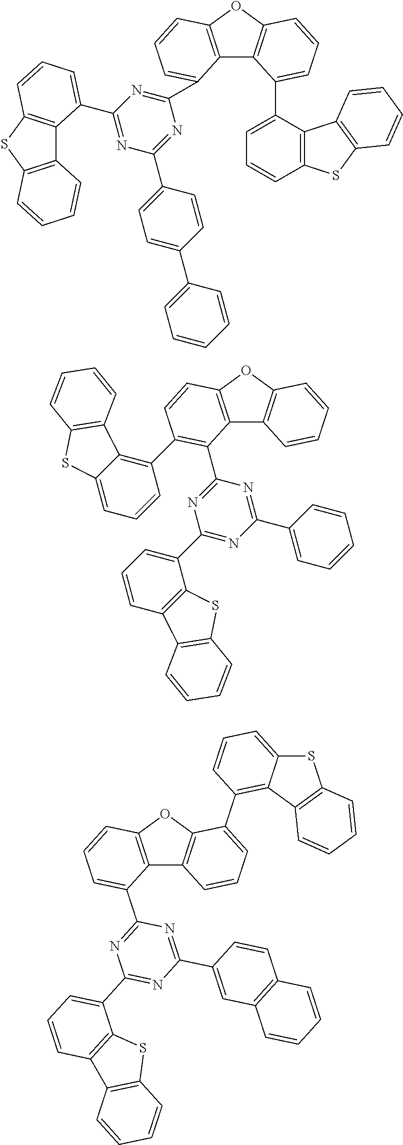

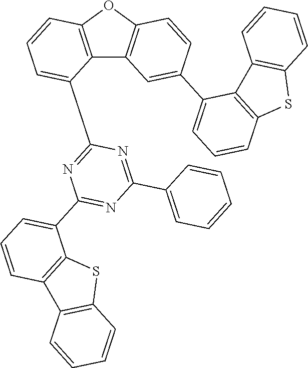 [SMILES: c1ccc(-c2nc(-c3cccc4c3sc3ccccc34)nc(-c3cccc4oc5ccc(-c6cccc7sc8ccccc8c67)cc5c34)n2)cc1]

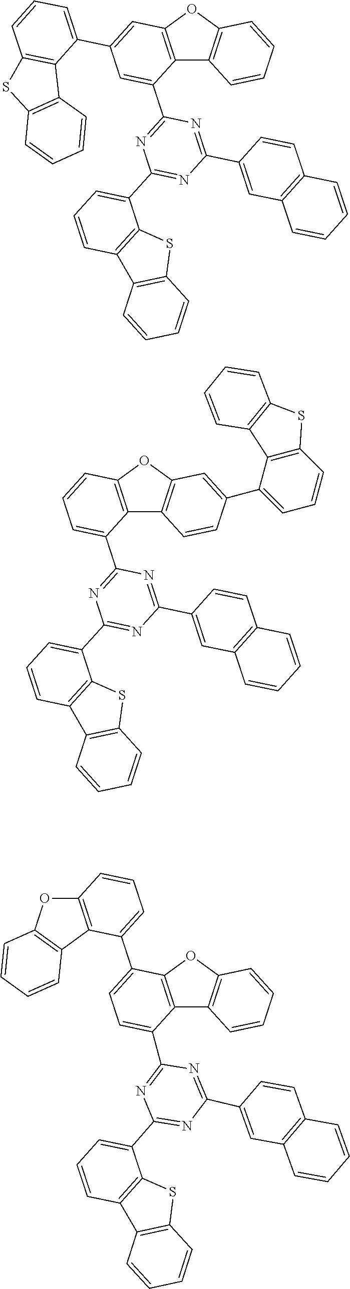 [SMILES: c1ccc2cc(-c3nc(-c4cccc5c4sc4ccccc45)nc(-c4cc(-c5cccc6sc7ccccc7c56)cc5oc6ccccc6c45)n3)ccc2c1 ; c1ccc2cc(-c3nc(-c4cccc5c4sc4ccccc45)nc(-c4cccc5oc6cc(-c7cccc8sc9ccccc9c78)ccc6c45)n3)ccc2c1 ; c1ccc2cc(-c3nc(-c4cccc5c4sc4ccccc45)nc(-c4ccc(-c5cccc6oc7ccccc7c56)c5oc6ccccc6c45)n3)ccc2c1]

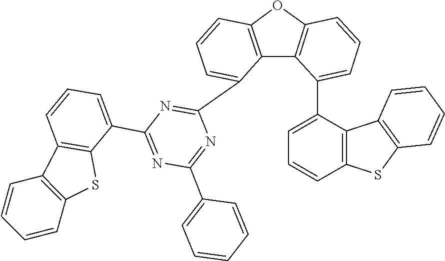 [SMILES: c1ccc(-c2nc(-c3cccc4c3sc3ccccc34)nc(-c3cccc4oc5cccc(-c6cccc7sc8ccccc8c67)c5c34)n2)cc1]

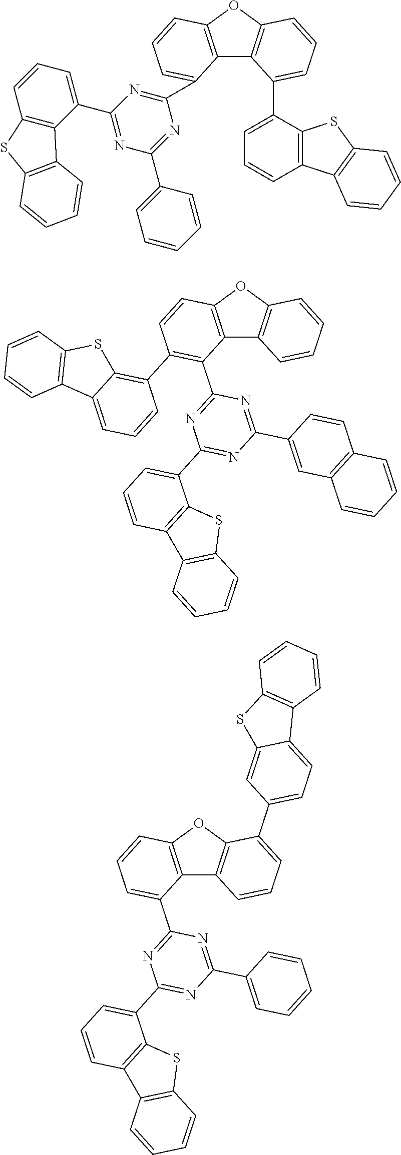 [SMILES: c1ccc(-c2nc(-c3cccc4sc5ccccc5c34)nc(-c3cccc4oc5cccc(-c6cccc7c6sc6ccccc67)c5c34)n2)cc1 ; c1ccc2cc(-c3nc(-c4cccc5c4sc4ccccc45)nc(-c4c(-c5cccc6c5sc5ccccc56)ccc5oc6ccccc6c45)n3)ccc2c1 ; c1ccc(-c2nc(-c3cccc4c3sc3ccccc34)nc(-c3cccc4oc5c(-c6ccc7c(c6)sc6ccccc67)cccc5c34)n2)cc1]

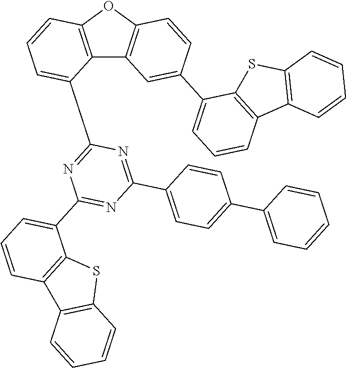 [SMILES: c1ccc(-c2ccc(-c3nc(-c4cccc5c4sc4ccccc45)nc(-c4cccc5oc6ccc(-c7cccc8c7sc7ccccc78)cc6c45)n3)cc2)cc1]

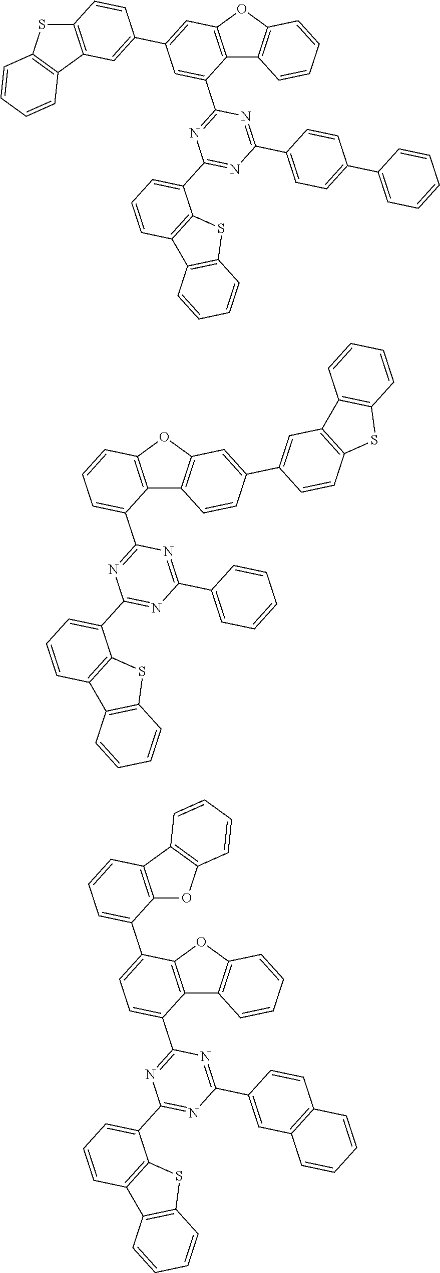 [SMILES: c1ccc(-c2ccc(-c3nc(-c4cccc5c4sc4ccccc45)nc(-c4cc(-c5ccc6sc7ccccc7c6c5)cc5oc6ccccc6c45)n3)cc2)cc1 ; c1ccc(-c2nc(-c3cccc4c3sc3ccccc34)nc(-c3cccc4oc5cc(-c6ccc7sc8ccccc8c7c6)ccc5c34)n2)cc1 ; c1ccc2cc(-c3nc(-c4cccc5c4sc4ccccc45)nc(-c4ccc(-c5cccc6c5oc5ccccc56)c5oc6ccccc6c45)n3)ccc2c1]

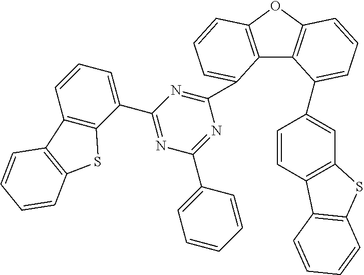 [SMILES: c1ccc(-c2nc(-c3cccc4c3sc3ccccc34)nc(-c3cccc4oc5cccc(-c6ccc7c(c6)sc6ccccc67)c5c34)n2)cc1]

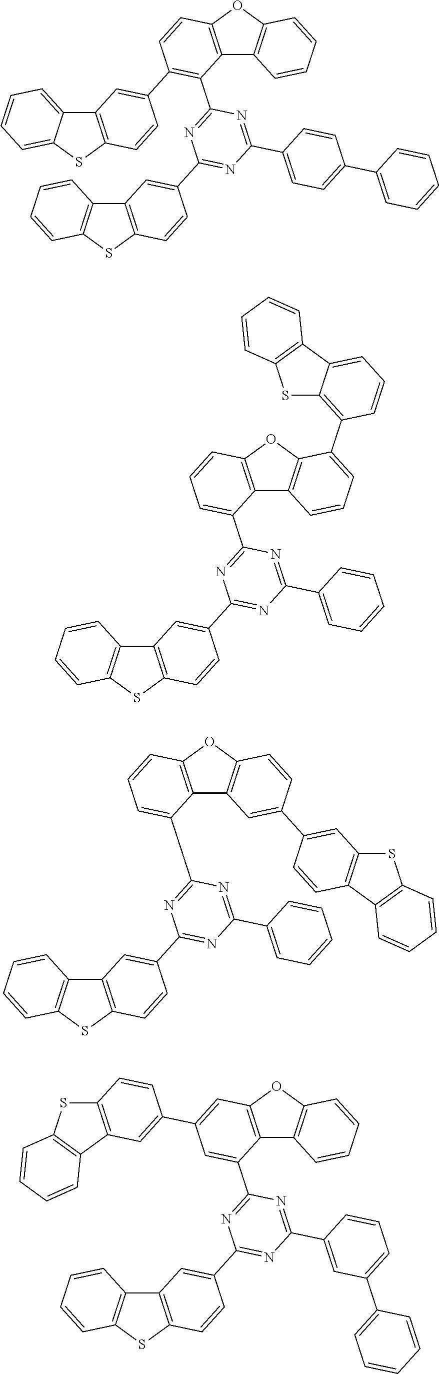 [SMILES: c1ccc(-c2ccc(-c3nc(-c4ccc5sc6ccccc6c5c4)nc(-c4c(-c5ccc6sc7ccccc7c6c5)ccc5oc6ccccc6c45)n3)cc2)cc1 ; c1ccc(-c2nc(-c3ccc4sc5ccccc5c4c3)nc(-c3cccc4oc5c(-c6cccc7c6sc6ccccc67)cccc5c34)n2)cc1 ; c1ccc(-c2nc(-c3ccc4sc5ccccc5c4c3)nc(-c3cccc4oc5ccc(-c6ccc7c(c6)sc6ccccc67)cc5c34)n2)cc1 ; c1ccc(-c2cccc(-c3nc(-c4ccc5sc6ccccc6c5c4)nc(-c4cc(-c5ccc6sc7ccccc7c6c5)cc5oc6ccccc6c45)n3)c2)cc1]

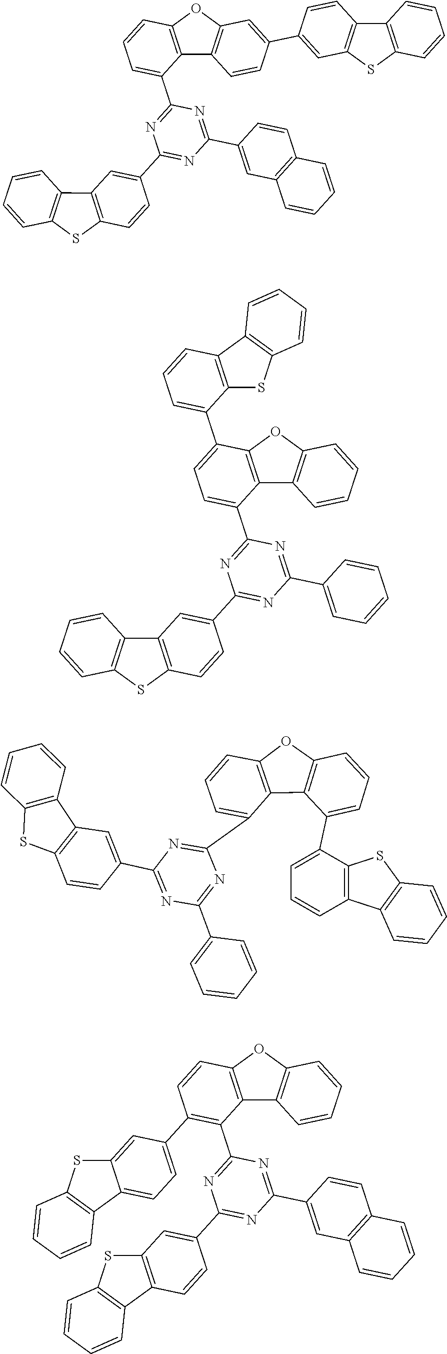 [SMILES: c1ccc2cc(-c3nc(-c4ccc5sc6ccccc6c5c4)nc(-c4cccc5oc6cc(-c7ccc8c(c7)sc7ccccc78)ccc6c45)n3)ccc2c1 ; c1ccc(-c2nc(-c3ccc4sc5ccccc5c4c3)nc(-c3ccc(-c4cccc5c4sc4ccccc45)c4oc5ccccc5c34)n2)cc1 ; c1ccc(-c2nc(-c3ccc4sc5ccccc5c4c3)nc(-c3cccc4oc5cccc(-c6cccc7c6sc6ccccc67)c5c34)n2)cc1 ; c1ccc2cc(-c3nc(-c4ccc5c(c4)sc4ccccc45)nc(-c4c(-c5ccc6c(c5)sc5ccccc56)ccc5oc6ccccc6c45)n3)ccc2c1]

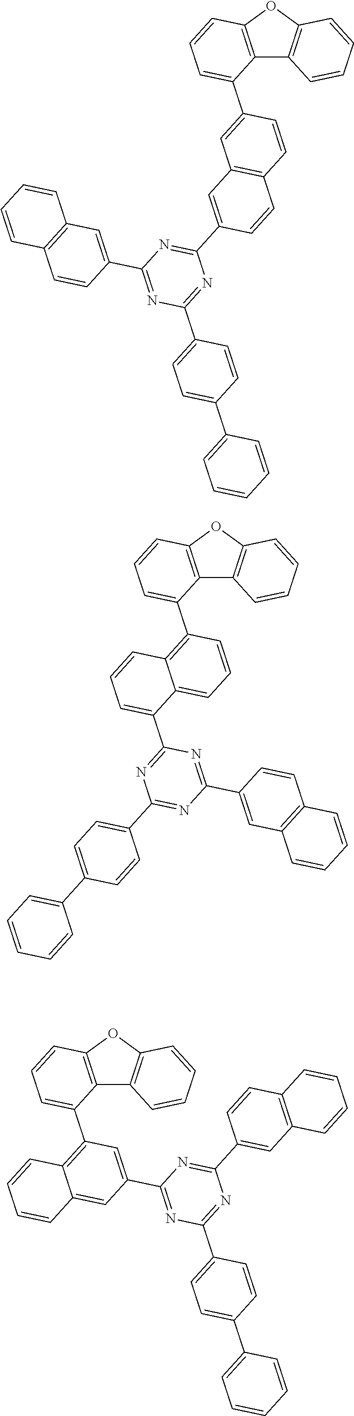

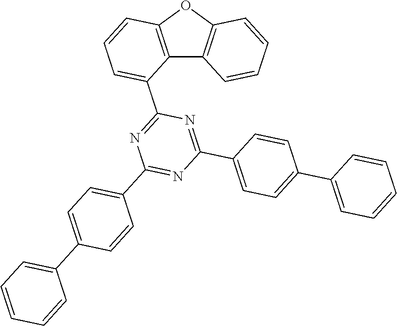 [SMILES: c1ccc(-c2ccc(-c3nc(-c4ccc(-c5ccccc5)cc4)nc(-c4cccc5oc6ccccc6c45)n3)cc2)cc1]

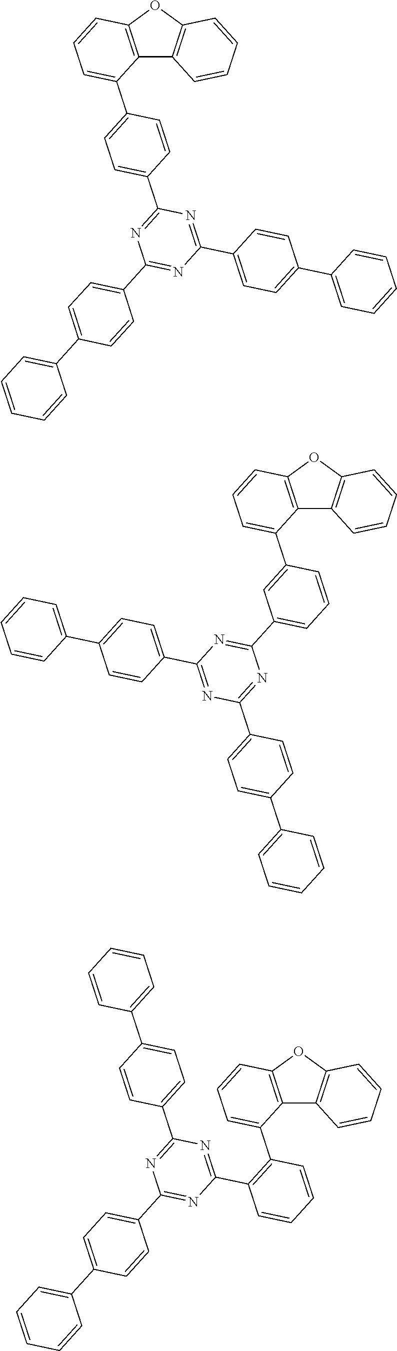 [SMILES: c1ccc(-c2ccc(-c3nc(-c4ccc(-c5ccccc5)cc4)nc(-c4ccc(-c5cccc6oc7ccccc7c56)cc4)n3)cc2)cc1 ; c1ccc(-c2ccc(-c3nc(-c4ccc(-c5ccccc5)cc4)nc(-c4cccc(-c5cccc6oc7ccccc7c56)c4)n3)cc2)cc1 ; c1ccc(-c2ccc(-c3nc(-c4ccc(-c5ccccc5)cc4)nc(-c4ccccc4-c4cccc5oc6ccccc6c45)n3)cc2)cc1]

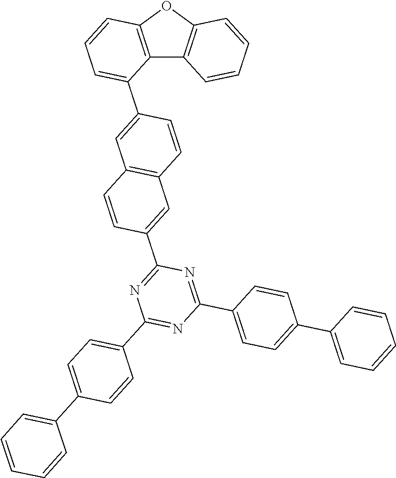 [SMILES: c1ccc(-c2ccc(-c3nc(-c4ccc(-c5ccccc5)cc4)nc(-c4ccc5cc(-c6cccc7oc8ccccc8c67)ccc5c4)n3)cc2)cc1]

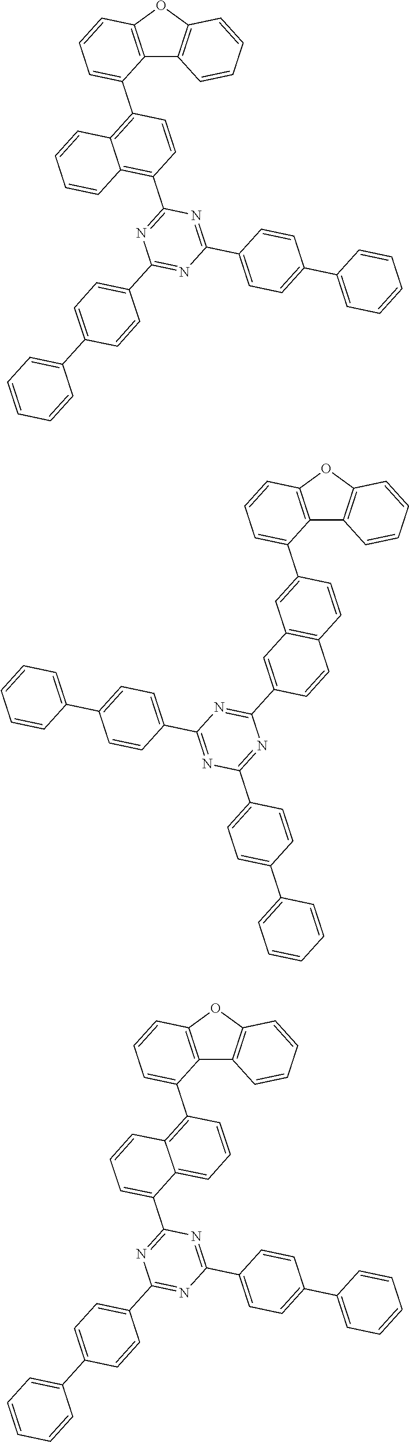 [SMILES: c1ccc(-c2ccc(-c3nc(-c4ccc(-c5ccccc5)cc4)nc(-c4ccc(-c5cccc6oc7ccccc7c56)c5ccccc45)n3)cc2)cc1 ; c1ccc(-c2ccc(-c3nc(-c4ccc(-c5ccccc5)cc4)nc(-c4ccc5ccc(-c6cccc7oc8ccccc8c67)cc5c4)n3)cc2)cc1 ; c1ccc(-c2ccc(-c3nc(-c4ccc(-c5ccccc5)cc4)nc(-c4cccc5c(-c6cccc7oc8ccccc8c67)cccc45)n3)cc2)cc1]

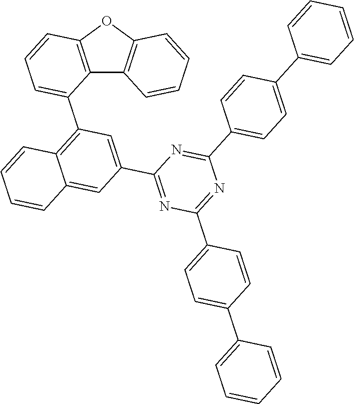 [SMILES: c1ccc(-c2ccc(-c3nc(-c4ccc(-c5ccccc5)cc4)nc(-c4cc(-c5cccc6oc7ccccc7c56)c5ccccc5c4)n3)cc2)cc1]

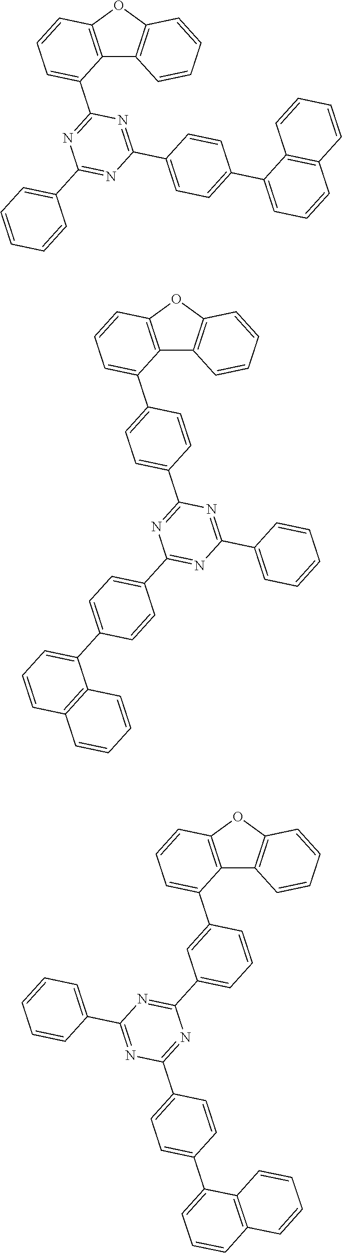 [SMILES: c1ccc(-c2nc(-c3ccc(-c4cccc5ccccc45)cc3)nc(-c3cccc4oc5ccccc5c34)n2)cc1 ; c1ccc(-c2nc(-c3ccc(-c4cccc5ccccc45)cc3)nc(-c3ccc(-c4cccc5oc6ccccc6c45)cc3)n2)cc1 ; c1ccc(-c2nc(-c3ccc(-c4cccc5ccccc45)cc3)nc(-c3cccc(-c4cccc5oc6ccccc6c45)c3)n2)cc1]

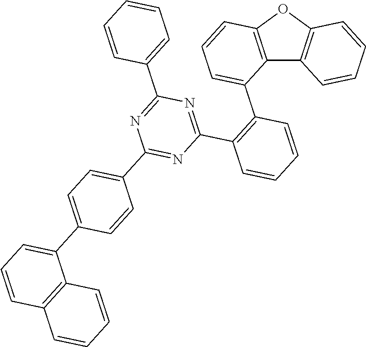 [SMILES: c1ccc(-c2nc(-c3ccc(-c4cccc5ccccc45)cc3)nc(-c3ccccc3-c3cccc4oc5ccccc5c34)n2)cc1]

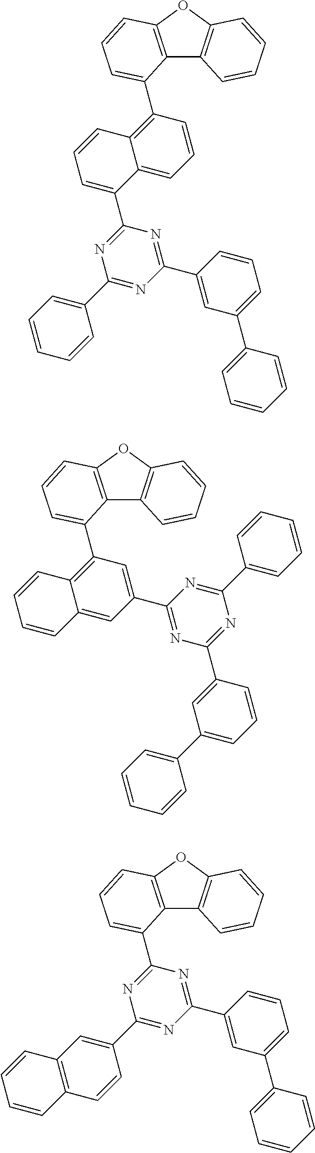 [SMILES: c1ccc(-c2cccc(-c3nc(-c4ccccc4)nc(-c4cccc5c(-c6cccc7oc8ccccc8c67)cccc45)n3)c2)cc1 ; c1ccc(-c2cccc(-c3nc(-c4ccccc4)nc(-c4cc(-c5cccc6oc7ccccc7c56)c5ccccc5c4)n3)c2)cc1 ; c1ccc(-c2cccc(-c3nc(-c4ccc5ccccc5c4)nc(-c4cccc5oc6ccccc6c45)n3)c2)cc1]

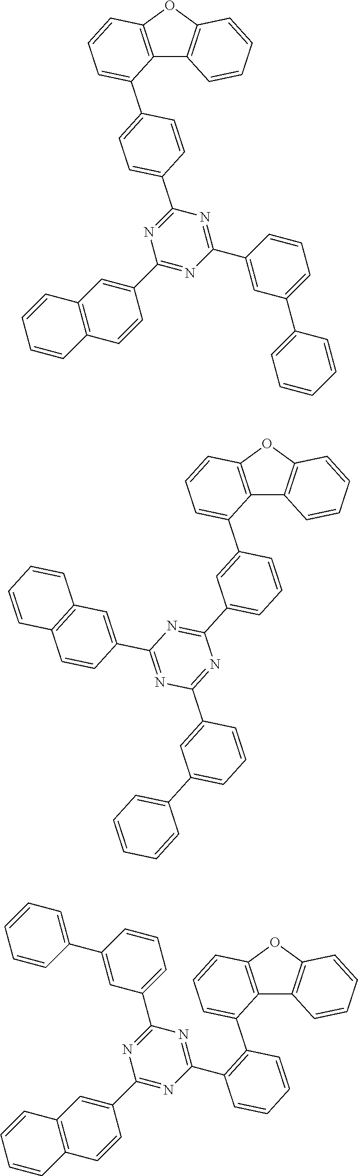 [SMILES: c1ccc(-c2cccc(-c3nc(-c4ccc(-c5cccc6oc7ccccc7c56)cc4)nc(-c4ccc5ccccc5c4)n3)c2)cc1 ; c1ccc(-c2cccc(-c3nc(-c4cccc(-c5cccc6oc7ccccc7c56)c4)nc(-c4ccc5ccccc5c4)n3)c2)cc1 ; c1ccc(-c2cccc(-c3nc(-c4ccc5ccccc5c4)nc(-c4ccccc4-c4cccc5oc6ccccc6c45)n3)c2)cc1]

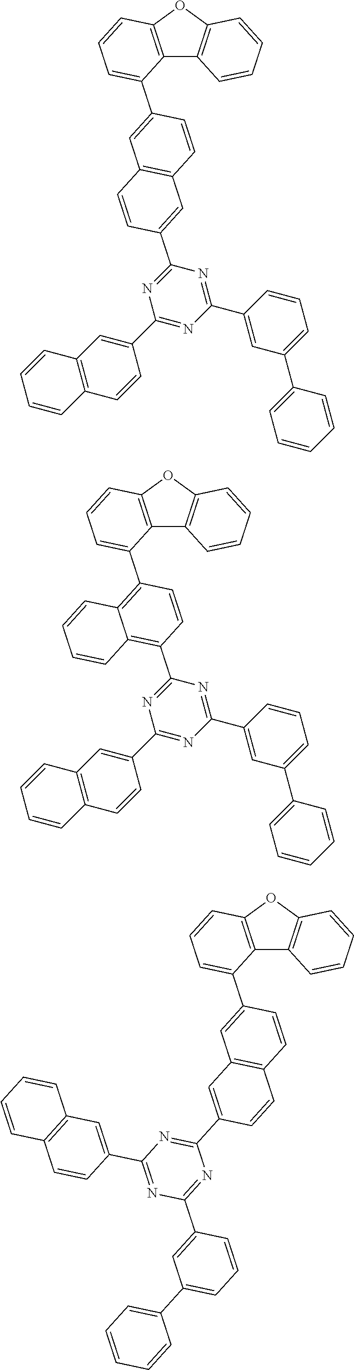

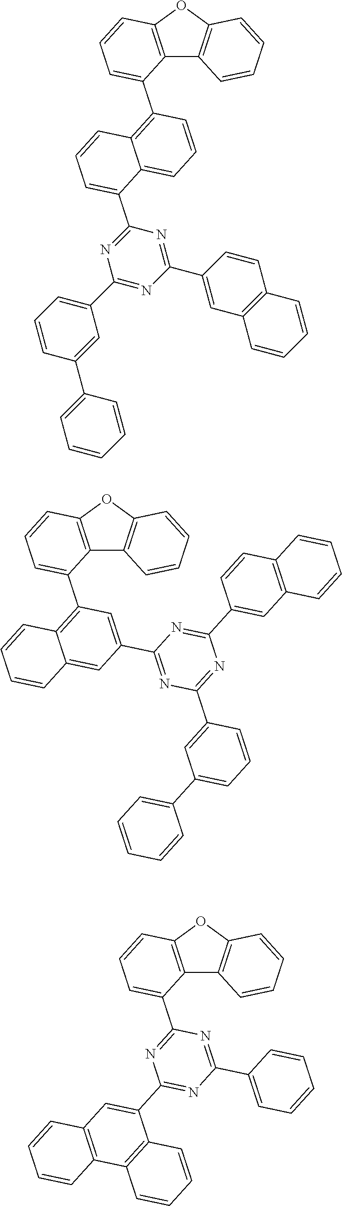

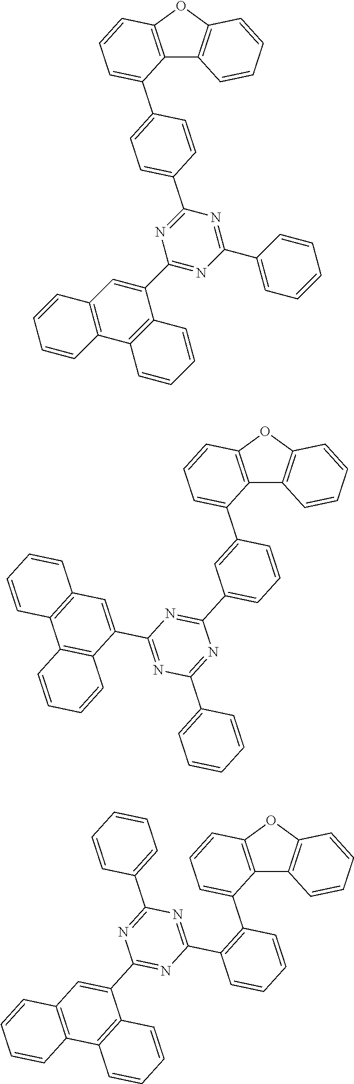 [SMILES: c1ccc(-c2nc(-c3ccc(-c4cccc5oc6ccccc6c45)cc3)nc(-c3cc4ccccc4c4ccccc34)n2)cc1 ; c1ccc(-c2nc(-c3cccc(-c4cccc5oc6ccccc6c45)c3)nc(-c3cc4ccccc4c4ccccc34)n2)cc1 ; c1ccc(-c2nc(-c3ccccc3-c3cccc4oc5ccccc5c34)nc(-c3cc4ccccc4c4ccccc34)n2)cc1]

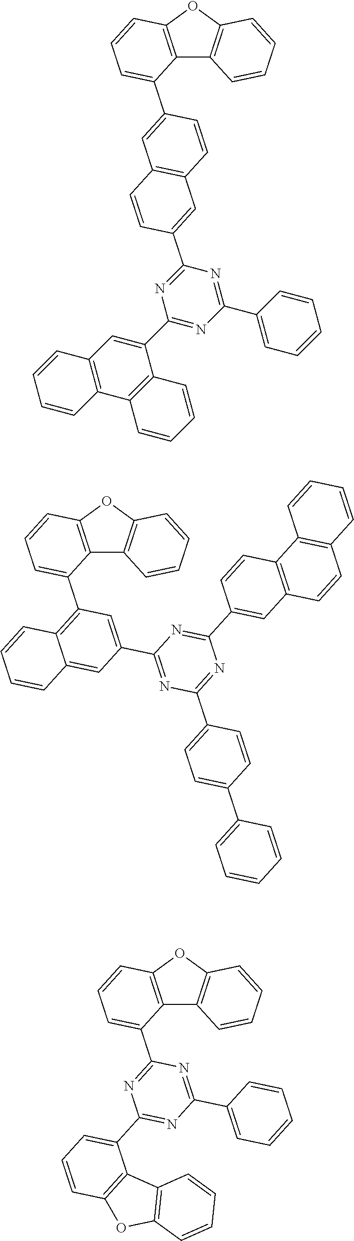 [SMILES: c1ccc(-c2nc(-c3ccc4cc(-c5cccc6oc7ccccc7c56)ccc4c3)nc(-c3cc4ccccc4c4ccccc34)n2)cc1 ; c1ccc(-c2ccc(-c3nc(-c4cc(-c5cccc6oc7ccccc7c56)c5ccccc5c4)nc(-c4ccc5c(ccc6ccccc65)c4)n3)cc2)cc1 ; c1ccc(-c2nc(-c3cccc4oc5ccccc5c34)nc(-c3cccc4oc5ccccc5c34)n2)cc1]

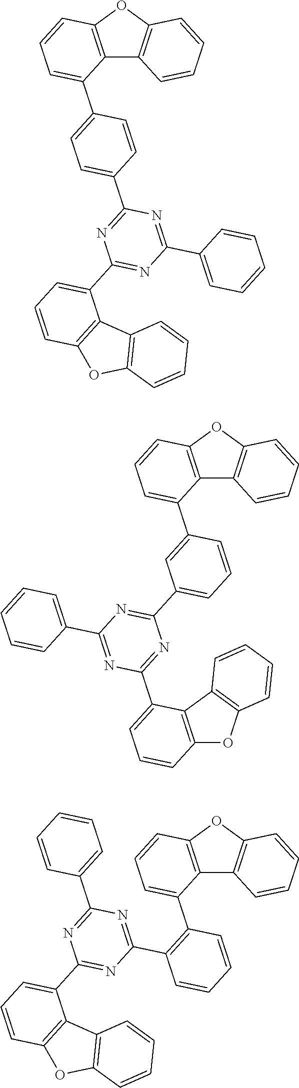 [SMILES: c1ccc(-c2nc(-c3ccc(-c4cccc5oc6ccccc6c45)cc3)nc(-c3cccc4oc5ccccc5c34)n2)cc1 ; c1ccc(-c2nc(-c3cccc(-c4cccc5oc6ccccc6c45)c3)nc(-c3cccc4oc5ccccc5c34)n2)cc1 ; c1ccc(-c2nc(-c3ccccc3-c3cccc4oc5ccccc5c34)nc(-c3cccc4oc5ccccc5c34)n2)cc1]

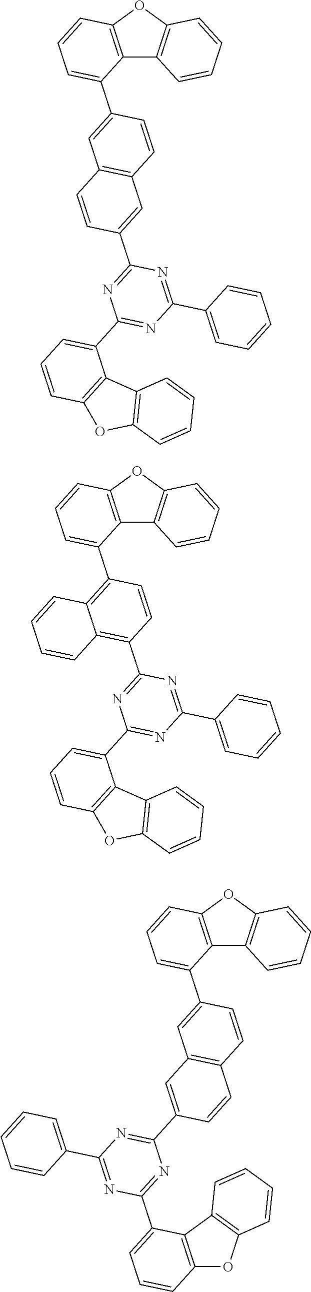

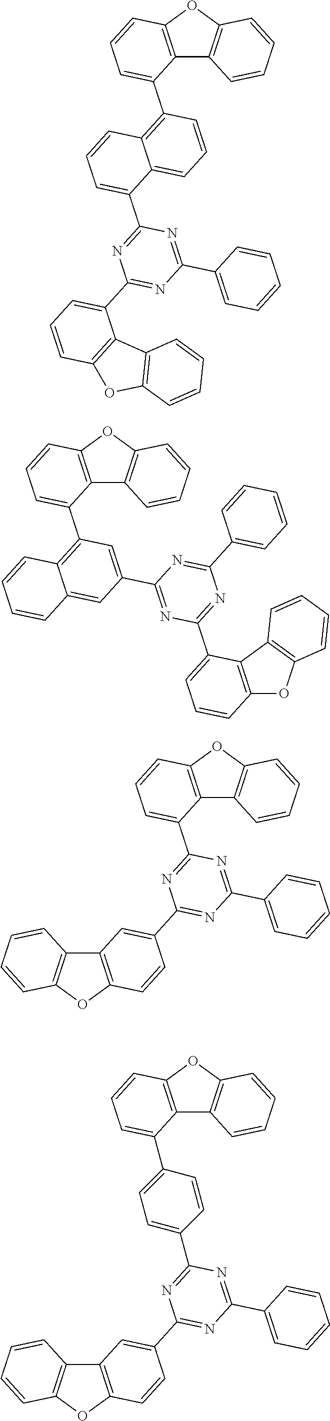

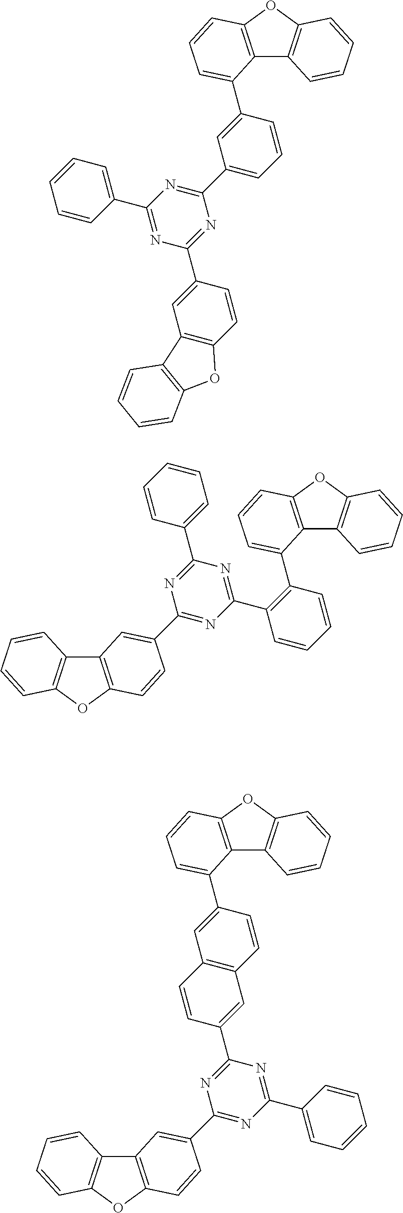

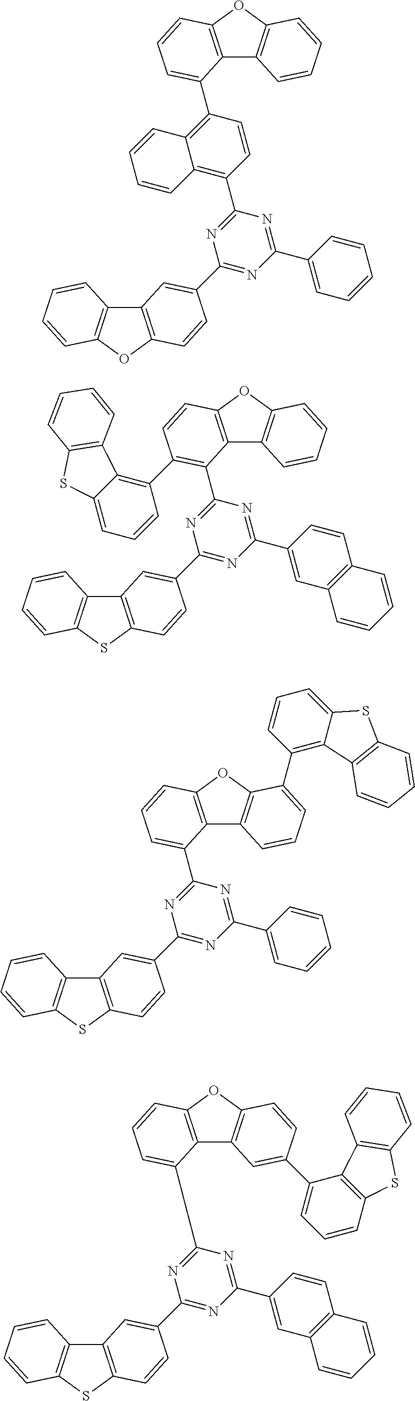

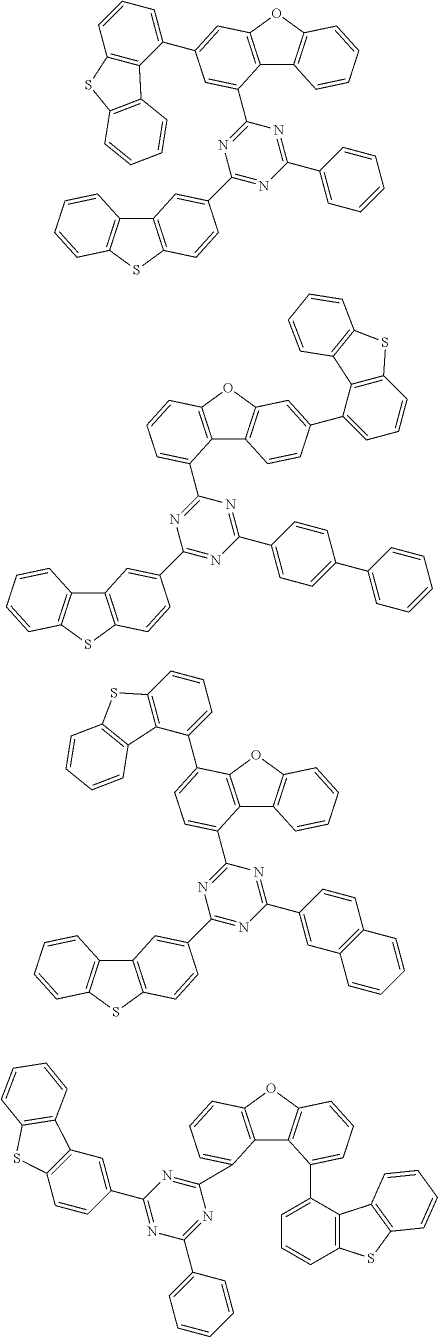

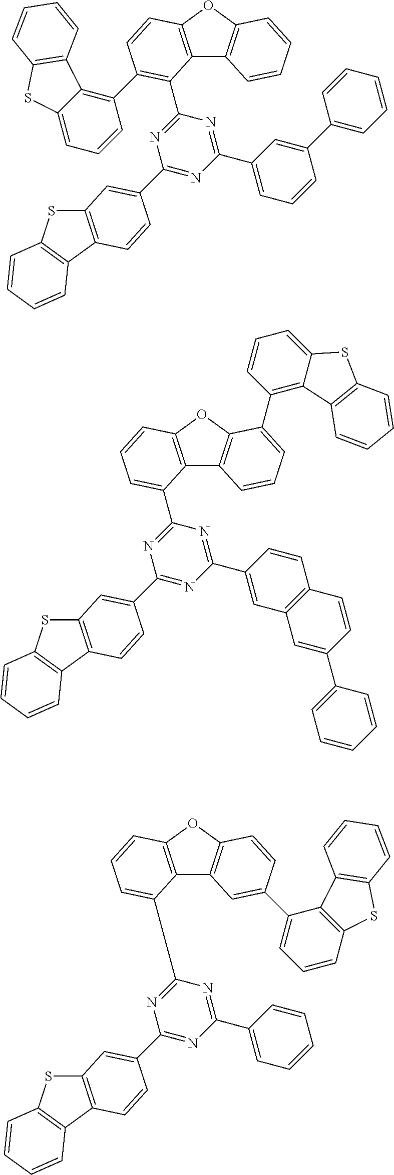 [SMILES: c1ccc(-c2cccc(-c3nc(-c4ccc5c(c4)sc4ccccc45)nc(-c4c(-c5cccc6sc7ccccc7c56)ccc5oc6ccccc6c45)n3)c2)cc1 ; c1ccc(-c2ccc3ccc(-c4nc(-c5ccc6c(c5)sc5ccccc56)nc(-c5cccc6oc7c(-c8cccc9sc%10ccccc%10c89)cccc7c56)n4)cc3c2)cc1 ; c1ccc(-c2nc(-c3ccc4c(c3)sc3ccccc34)nc(-c3cccc4oc5ccc(-c6cccc7sc8ccccc8c67)cc5c34)n2)cc1]

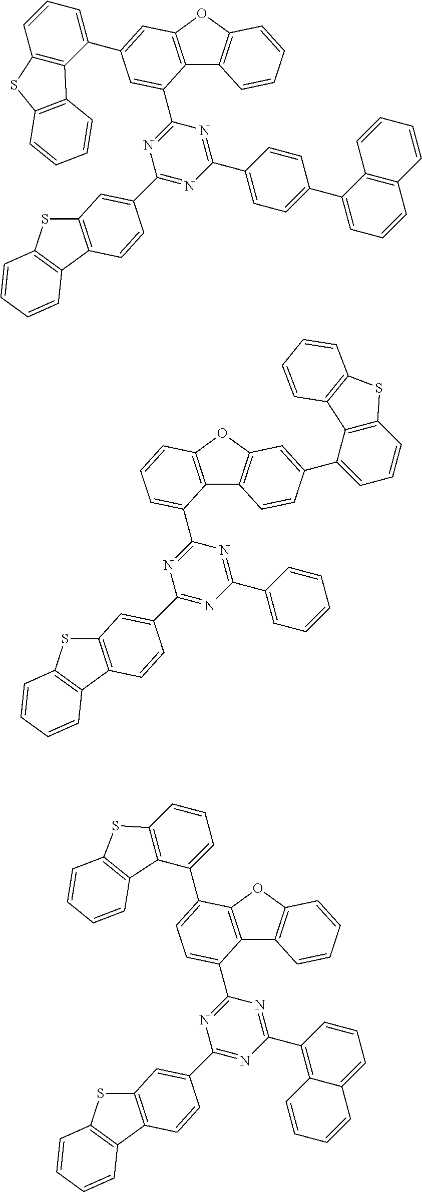 [SMILES: c1ccc2c(-c3ccc(-c4nc(-c5ccc6c(c5)sc5ccccc56)nc(-c5cc(-c6cccc7sc8ccccc8c67)cc6oc7ccccc7c56)n4)cc3)cccc2c1 ; c1ccc(-c2nc(-c3ccc4c(c3)sc3ccccc34)nc(-c3cccc4oc5cc(-c6cccc7sc8ccccc8c67)ccc5c34)n2)cc1 ; c1ccc2c(-c3nc(-c4ccc5c(c4)sc4ccccc45)nc(-c4ccc(-c5cccc6sc7ccccc7c56)c5oc6ccccc6c45)n3)cccc2c1]

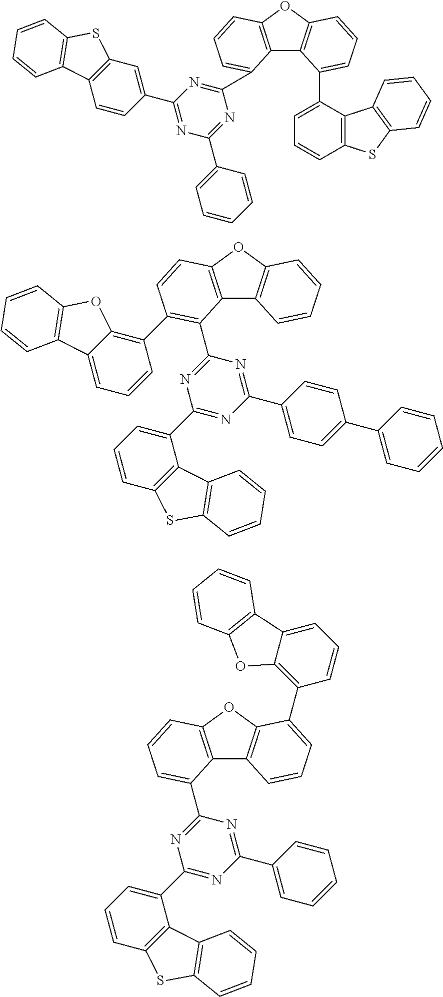

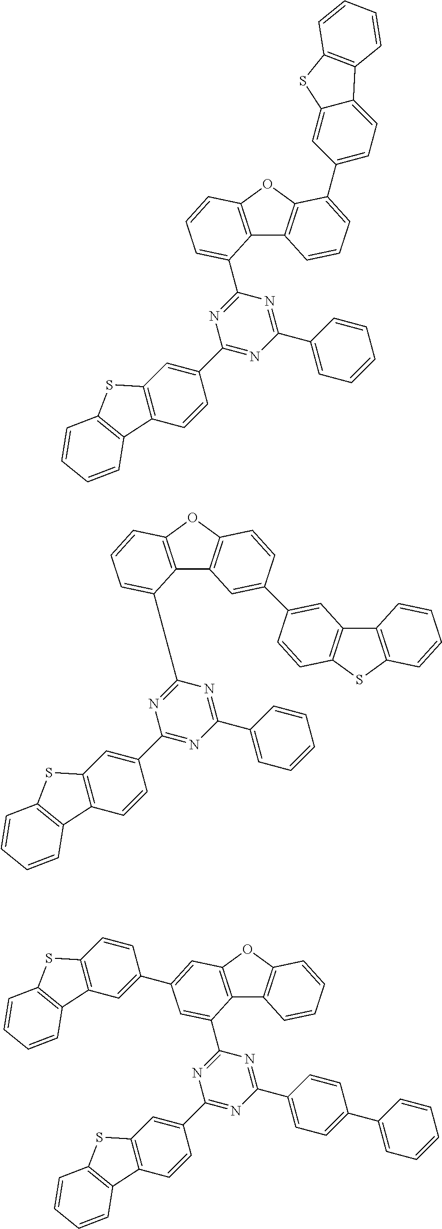 [SMILES: c1ccc(-c2nc(-c3ccc4c(c3)sc3ccccc34)nc(-c3cccc4oc5c(-c6ccc7c(c6)sc6ccccc67)cccc5c34)n2)cc1 ; c1ccc(-c2nc(-c3ccc4c(c3)sc3ccccc34)nc(-c3cccc4oc5ccc(-c6ccc7sc8ccccc8c7c6)cc5c34)n2)cc1 ; c1ccc(-c2ccc(-c3nc(-c4ccc5c(c4)sc4ccccc45)nc(-c4cc(-c5ccc6sc7ccccc7c6c5)cc5oc6ccccc6c45)n3)cc2)cc1]

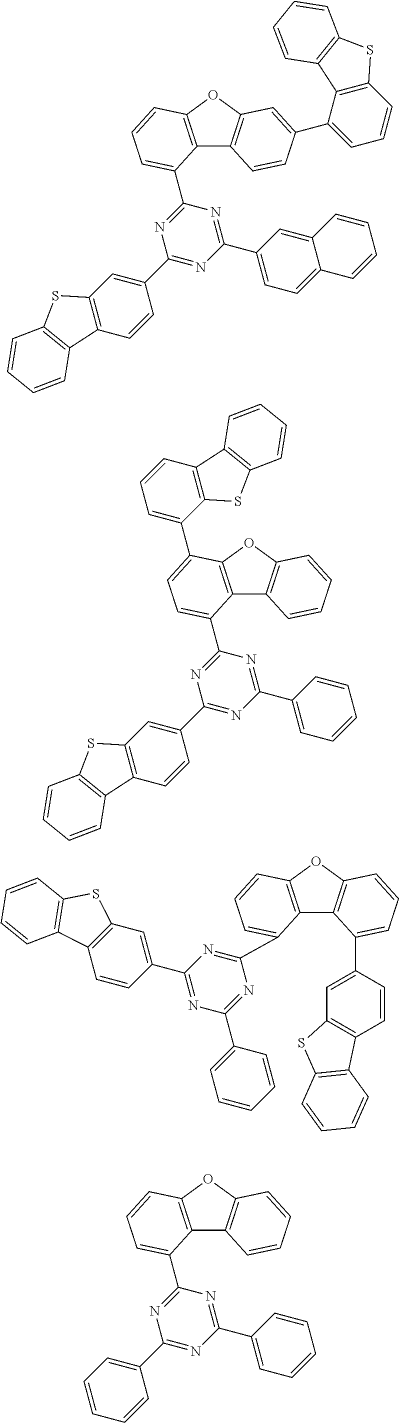 [SMILES: c1ccc2cc(-c3nc(-c4ccc5c(c4)sc4ccccc45)nc(-c4cccc5oc6cc(-c7cccc8sc9ccccc9c78)ccc6c45)n3)ccc2c1 ; c1ccc(-c2nc(-c3ccc4c(c3)sc3ccccc34)nc(-c3ccc(-c4cccc5c4sc4ccccc45)c4oc5ccccc5c34)n2)cc1 ; c1ccc(-c2nc(-c3ccc4c(c3)sc3ccccc34)nc(-c3cccc4oc5cccc(-c6ccc7c(c6)sc6ccccc67)c5c34)n2)cc1 ; c1ccc(-c2nc(-c3ccccc3)nc(-c3cccc4oc5ccccc5c34)n2)cc1]

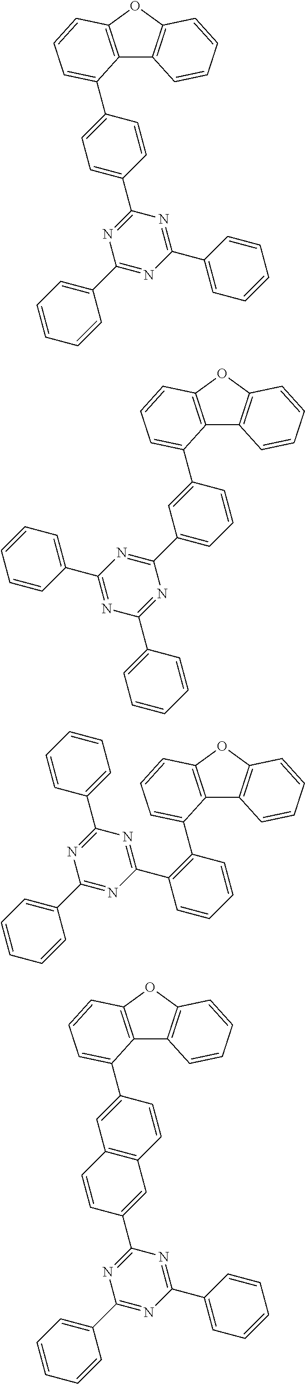

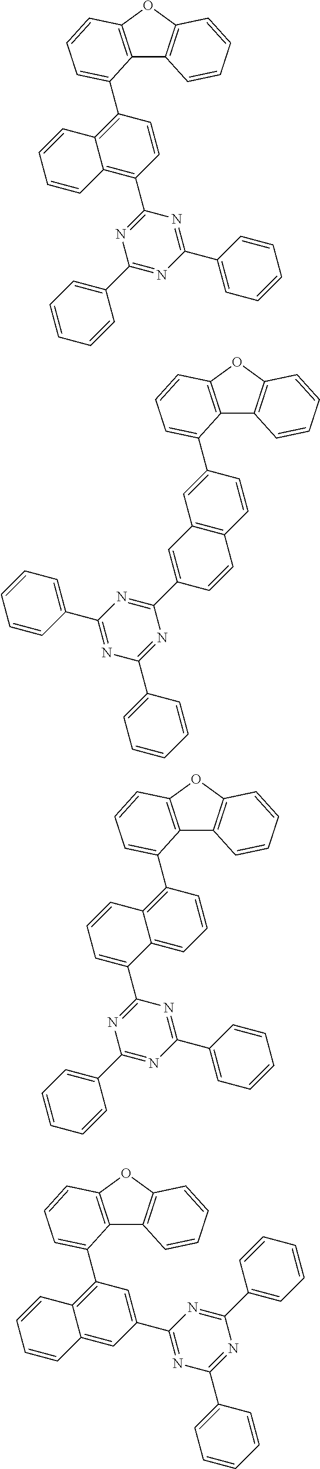

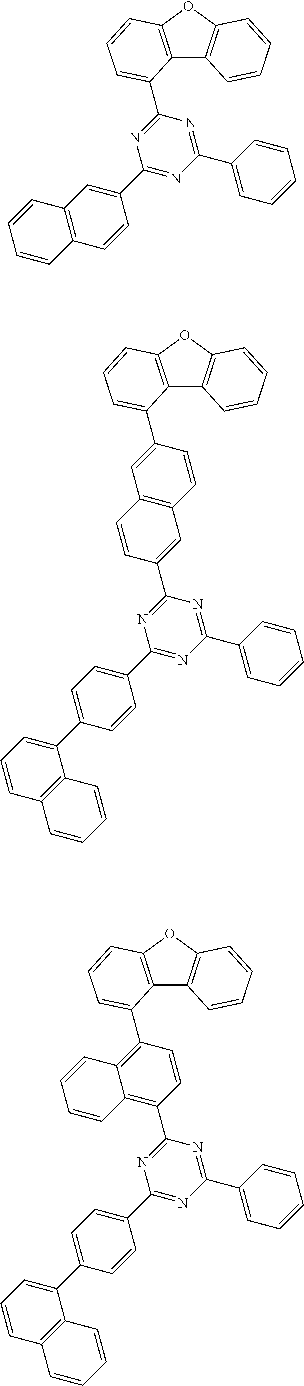 [SMILES: c1ccc(-c2nc(-c3ccc4ccccc4c3)nc(-c3cccc4oc5ccccc5c34)n2)cc1 ; c1ccc(-c2nc(-c3ccc(-c4cccc5ccccc45)cc3)nc(-c3ccc4cc(-c5cccc6oc7ccccc7c56)ccc4c3)n2)cc1 ; c1ccc(-c2nc(-c3ccc(-c4cccc5ccccc45)cc3)nc(-c3ccc(-c4cccc5oc6ccccc6c45)c4ccccc34)n2)cc1]

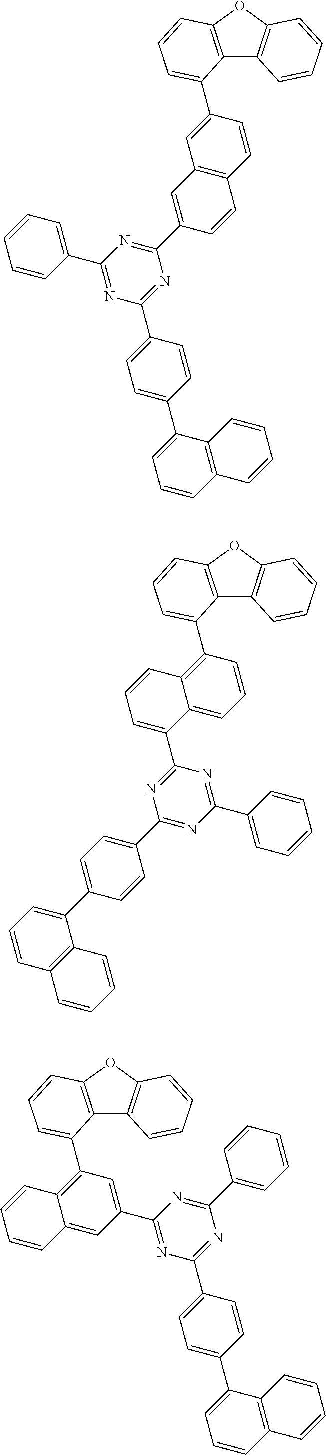 [SMILES: c1ccc(-c2nc(-c3ccc(-c4cccc5ccccc45)cc3)nc(-c3ccc4ccc(-c5cccc6oc7ccccc7c56)cc4c3)n2)cc1 ; c1ccc(-c2nc(-c3ccc(-c4cccc5ccccc45)cc3)nc(-c3cccc4c(-c5cccc6oc7ccccc7c56)cccc34)n2)cc1 ; c1ccc(-c2nc(-c3ccc(-c4cccc5ccccc45)cc3)nc(-c3cc(-c4cccc5oc6ccccc6c45)c4ccccc4c3)n2)cc1]

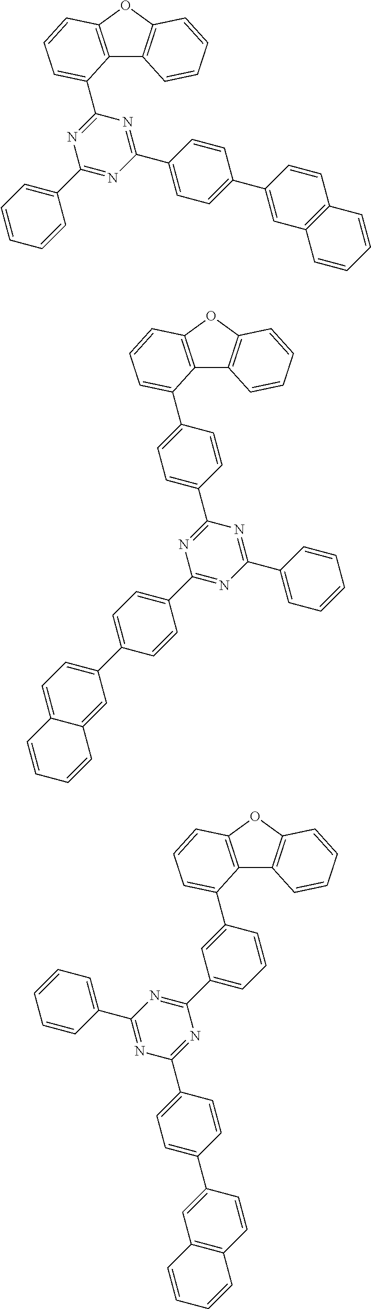

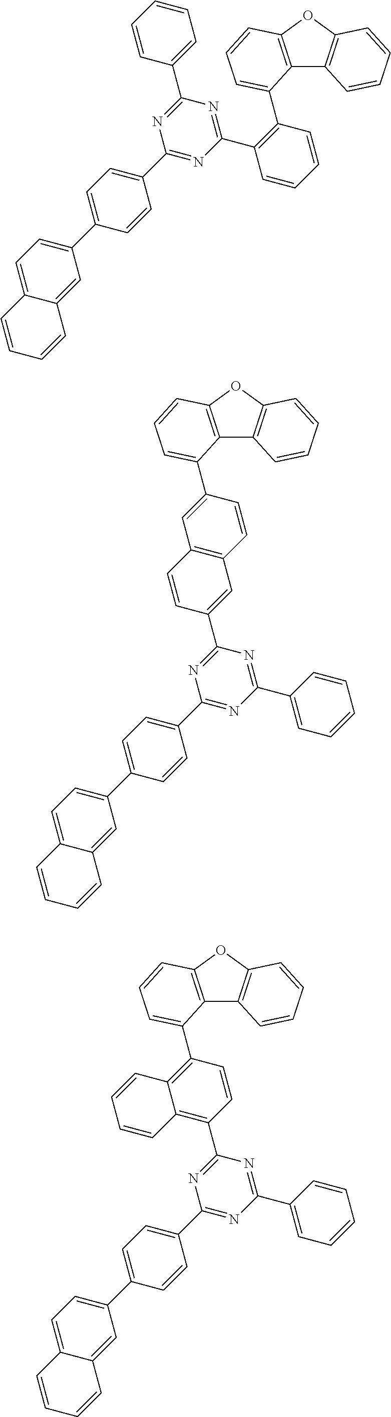 [SMILES: c1ccc(-c2nc(-c3ccc(-c4ccc5ccccc5c4)cc3)nc(-c3ccccc3-c3cccc4oc5ccccc5c34)n2)cc1 ; c1ccc(-c2nc(-c3ccc(-c4ccc5ccccc5c4)cc3)nc(-c3ccc4cc(-c5cccc6oc7ccccc7c56)ccc4c3)n2)cc1 ; c1ccc(-c2nc(-c3ccc(-c4ccc5ccccc5c4)cc3)nc(-c3ccc(-c4cccc5oc6ccccc6c45)c4ccccc34)n2)cc1]

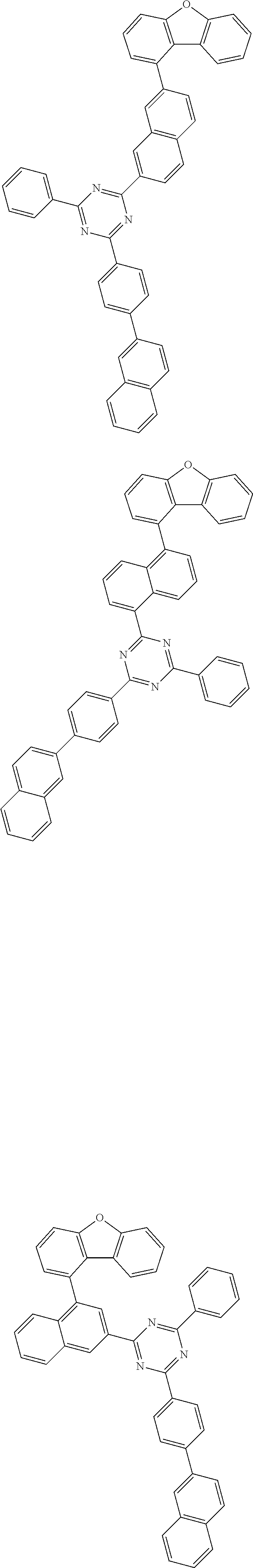 [SMILES: c1ccc(-c2nc(-c3ccc(-c4ccc5ccccc5c4)cc3)nc(-c3ccc4ccc(-c5cccc6oc7ccccc7c56)cc4c3)n2)cc1 ; c1ccc(-c2nc(-c3ccc(-c4ccc5ccccc5c4)cc3)nc(-c3cccc4c(-c5cccc6oc7ccccc7c56)cccc34)n2)cc1 ; c1ccc(-c2nc(-c3ccc(-c4ccc5ccccc5c4)cc3)nc(-c3cc(-c4cccc5oc6ccccc6c45)c4ccccc4c3)n2)cc1]

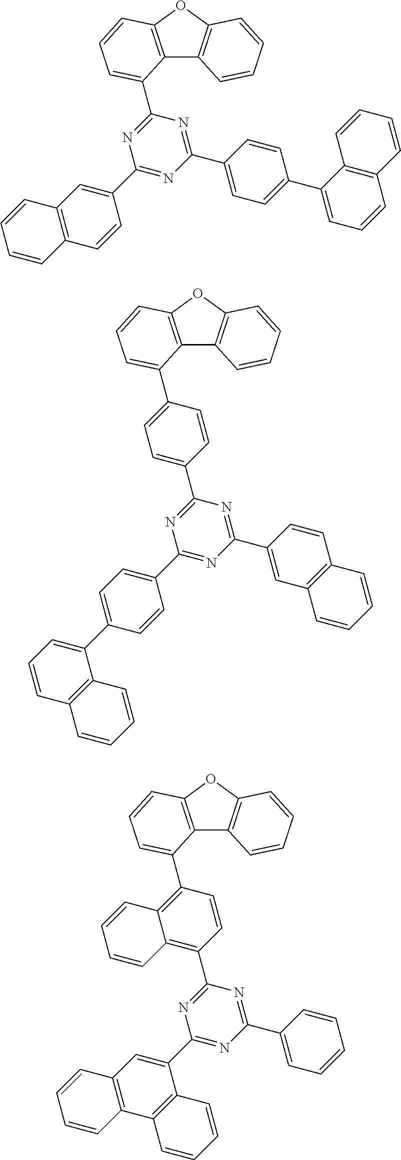 [SMILES: c1ccc2cc(-c3nc(-c4ccc(-c5cccc6ccccc56)cc4)nc(-c4cccc5oc6ccccc6c45)n3)ccc2c1 ; c1ccc2cc(-c3nc(-c4ccc(-c5cccc6ccccc56)cc4)nc(-c4ccc(-c5cccc6oc7ccccc7c56)cc4)n3)ccc2c1 ; c1ccc(-c2nc(-c3ccc(-c4cccc5oc6ccccc6c45)c4ccccc34)nc(-c3cc4ccccc4c4ccccc34)n2)cc1]

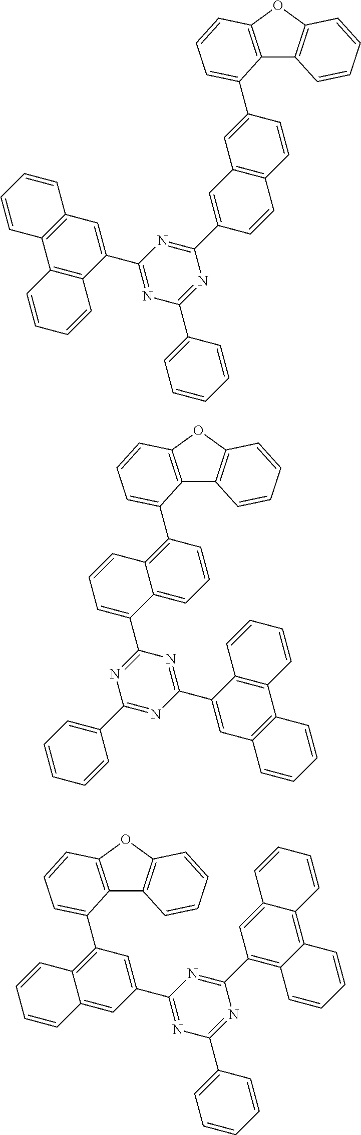

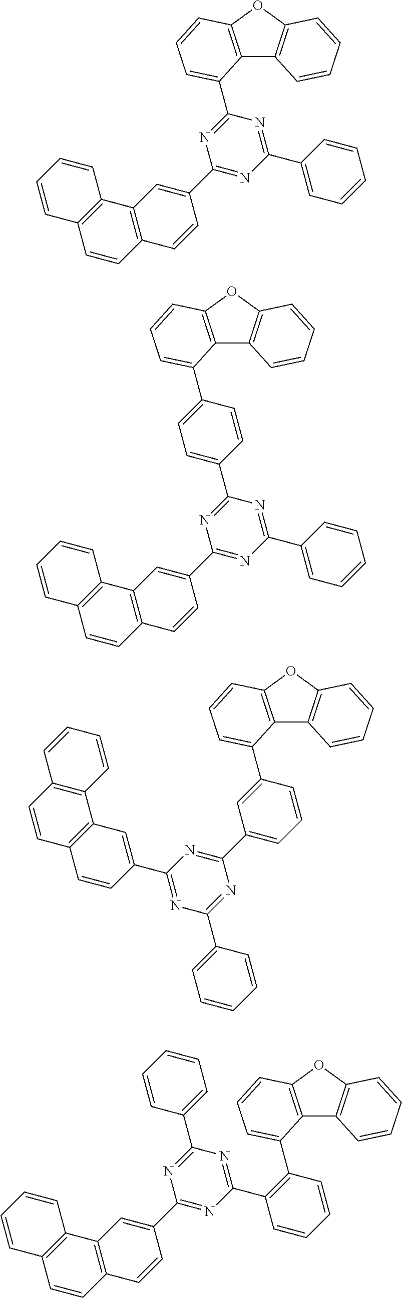 [SMILES: c1ccc(-c2nc(-c3ccc4ccc5ccccc5c4c3)nc(-c3cccc4oc5ccccc5c34)n2)cc1 ; c1ccc(-c2nc(-c3ccc(-c4cccc5oc6ccccc6c45)cc3)nc(-c3ccc4ccc5ccccc5c4c3)n2)cc1 ; c1ccc(-c2nc(-c3cccc(-c4cccc5oc6ccccc6c45)c3)nc(-c3ccc4ccc5ccccc5c4c3)n2)cc1 ; c1ccc(-c2nc(-c3ccc4ccc5ccccc5c4c3)nc(-c3ccccc3-c3cccc4oc5ccccc5c34)n2)cc1]

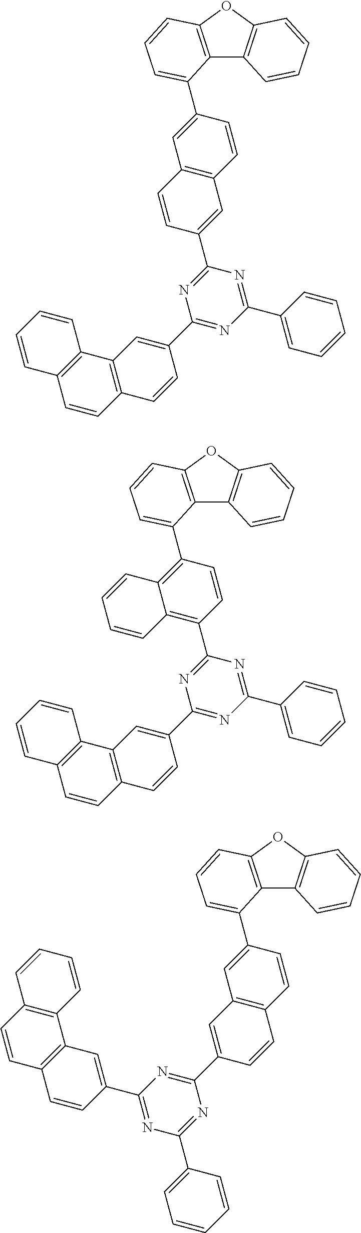 [SMILES: c1ccc(-c2nc(-c3ccc4cc(-c5cccc6oc7ccccc7c56)ccc4c3)nc(-c3ccc4ccc5ccccc5c4c3)n2)cc1 ; c1ccc(-c2nc(-c3ccc4ccc5ccccc5c4c3)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4ccc5ccccc5c4c3)n2)cc1]

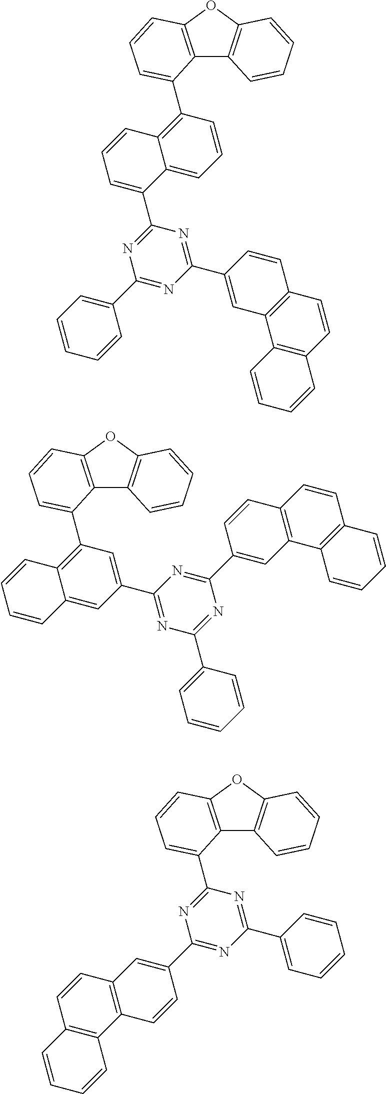 [SMILES: c1ccc(-c2nc(-c3ccc4ccc5ccccc5c4c3)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4ccc5ccccc5c4c3)n2)cc1 ; c1ccc(-c2nc(-c3ccc4c(ccc5ccccc54)c3)nc(-c3cccc4oc5ccccc5c34)n2)cc1]

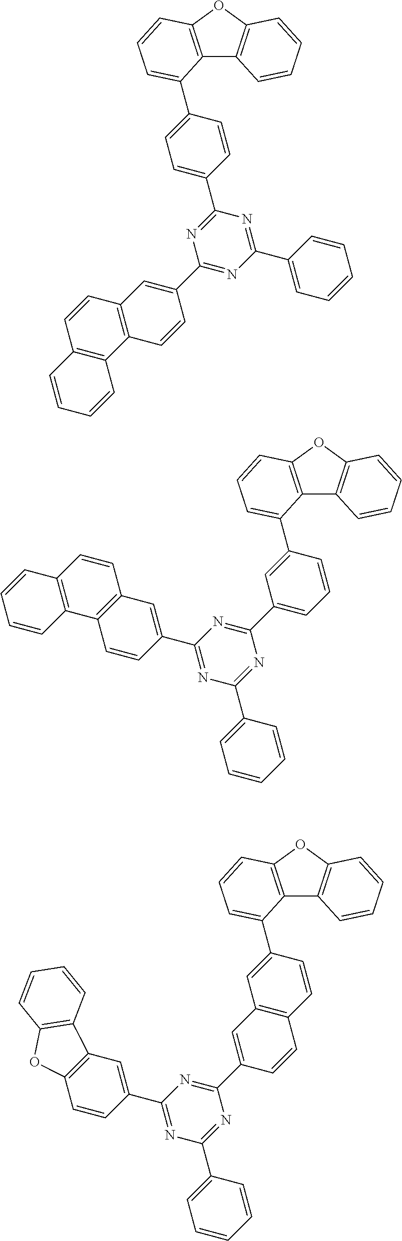 [SMILES: c1ccc(-c2nc(-c3ccc(-c4cccc5oc6ccccc6c45)cc3)nc(-c3ccc4c(ccc5ccccc54)c3)n2)cc1 ; c1ccc(-c2nc(-c3cccc(-c4cccc5oc6ccccc6c45)c3)nc(-c3ccc4c(ccc5ccccc54)c3)n2)cc1 ; c1ccc(-c2nc(-c3ccc4ccc(-c5cccc6oc7ccccc7c56)cc4c3)nc(-c3ccc4oc5ccccc5c4c3)n2)cc1]

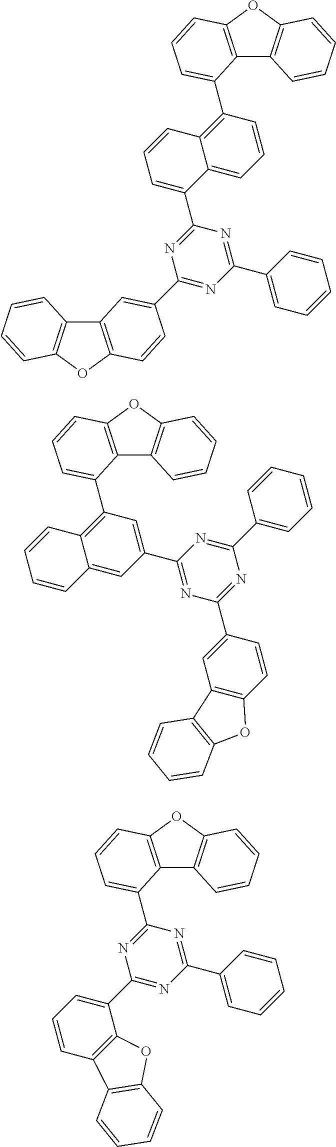

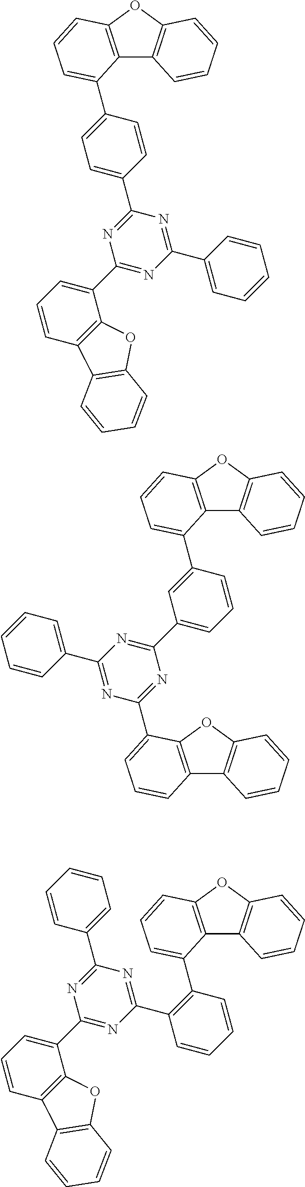 [SMILES: c1ccc(-c2nc(-c3ccc(-c4cccc5oc6ccccc6c45)cc3)nc(-c3cccc4c3oc3ccccc34)n2)cc1 ; c1ccc(-c2nc(-c3cccc(-c4cccc5oc6ccccc6c45)c3)nc(-c3cccc4c3oc3ccccc34)n2)cc1 ; c1ccc(-c2nc(-c3ccccc3-c3cccc4oc5ccccc5c34)nc(-c3cccc4c3oc3ccccc34)n2)cc1]

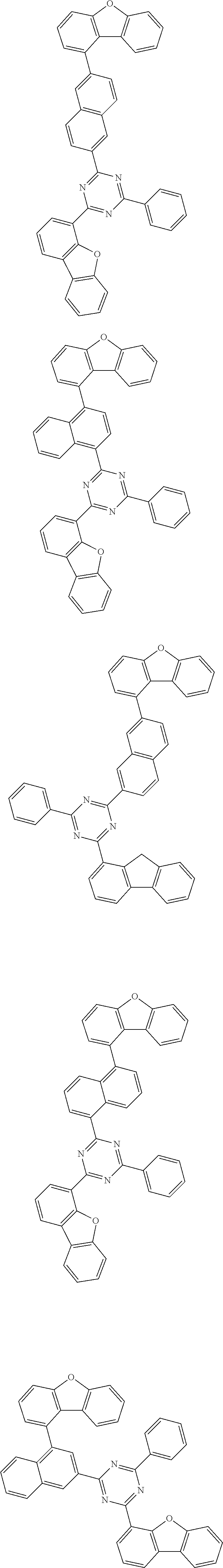

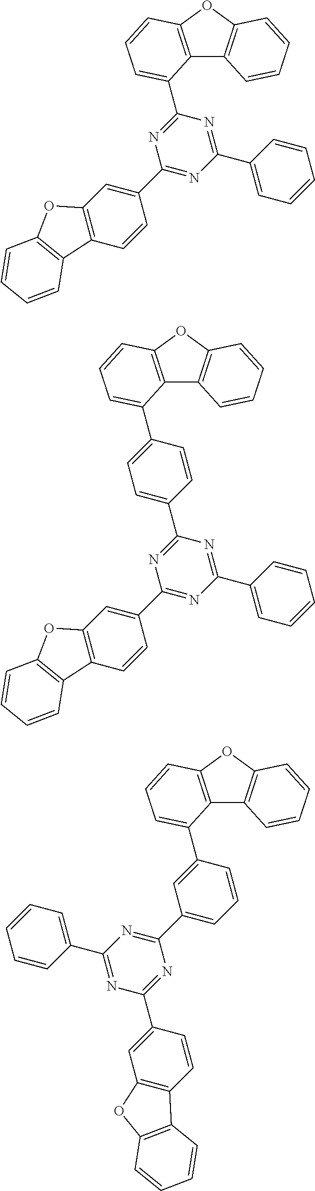 [SMILES: c1ccc(-c2nc(-c3ccc4c(c3)oc3ccccc34)nc(-c3cccc4oc5ccccc5c34)n2)cc1 ; c1ccc(-c2nc(-c3ccc(-c4cccc5oc6ccccc6c45)cc3)nc(-c3ccc4c(c3)oc3ccccc34)n2)cc1 ; c1ccc(-c2nc(-c3cccc(-c4cccc5oc6ccccc6c45)c3)nc(-c3ccc4c(c3)oc3ccccc34)n2)cc1]

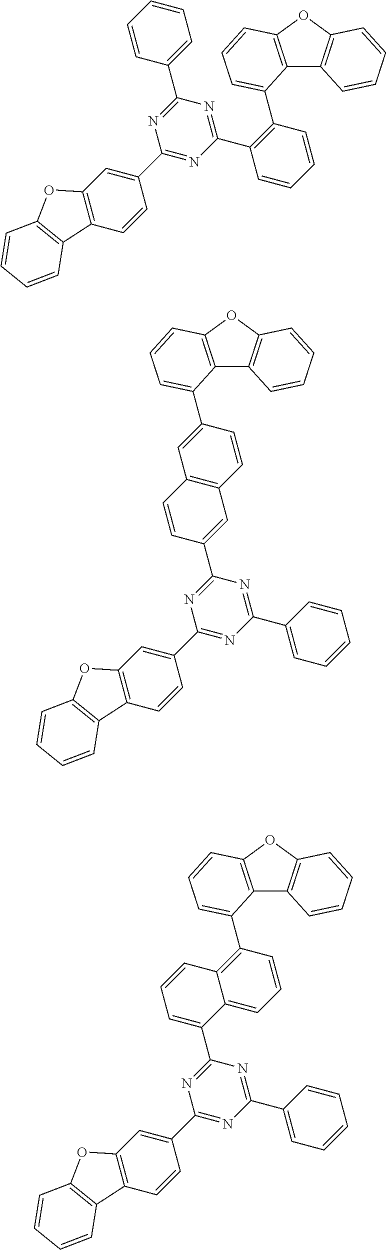 [SMILES: c1ccc(-c2nc(-c3ccc4c(c3)oc3ccccc34)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4c(c3)oc3ccccc34)n2)cc1 ; c1ccc(-c2nc(-c3ccc4c(c3)oc3ccccc34)nc(-c3cccc4c(-c5cccc6oc7ccccc7c56)cccc34)n2)cc1]

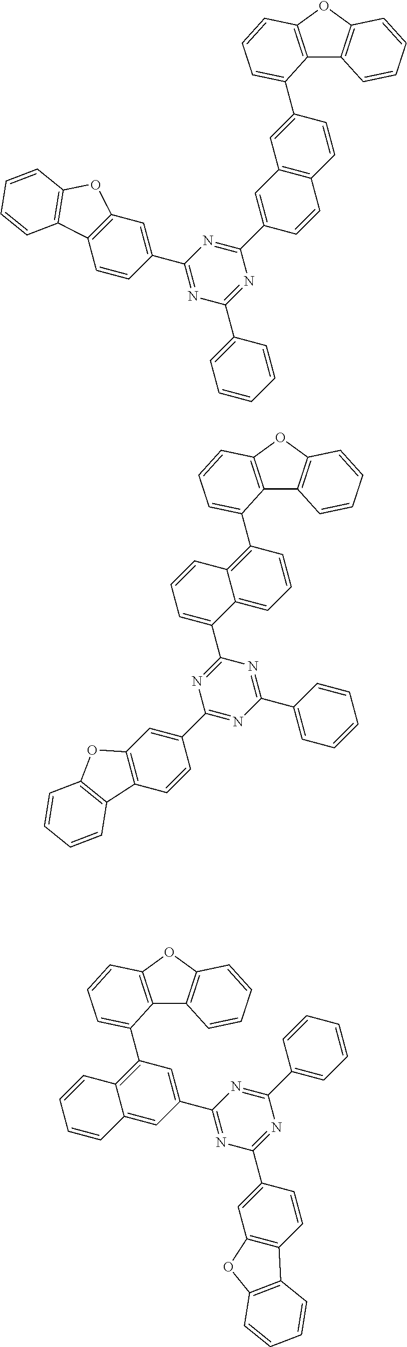

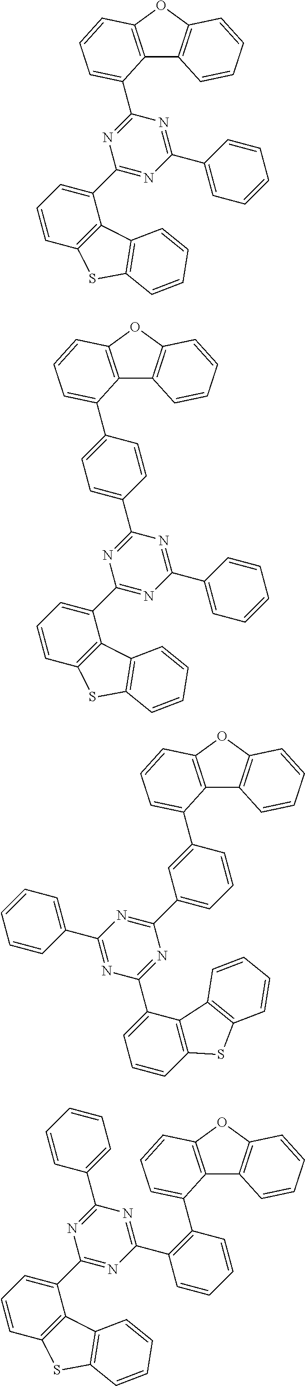

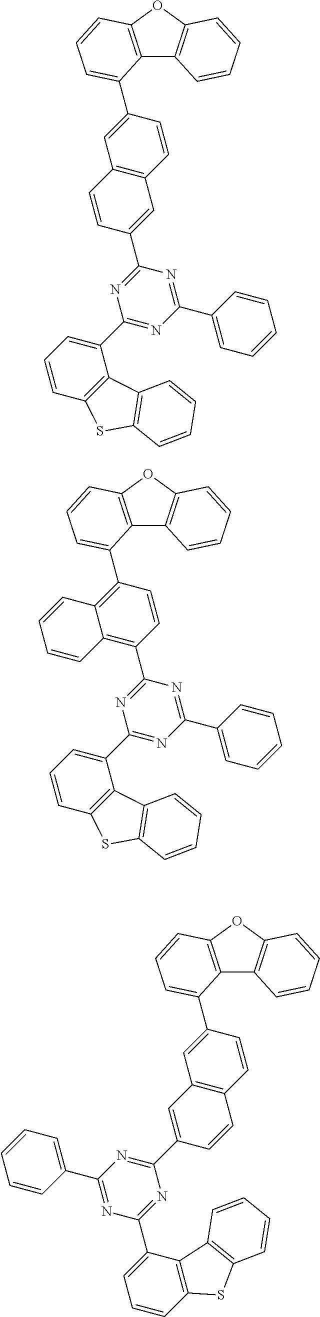

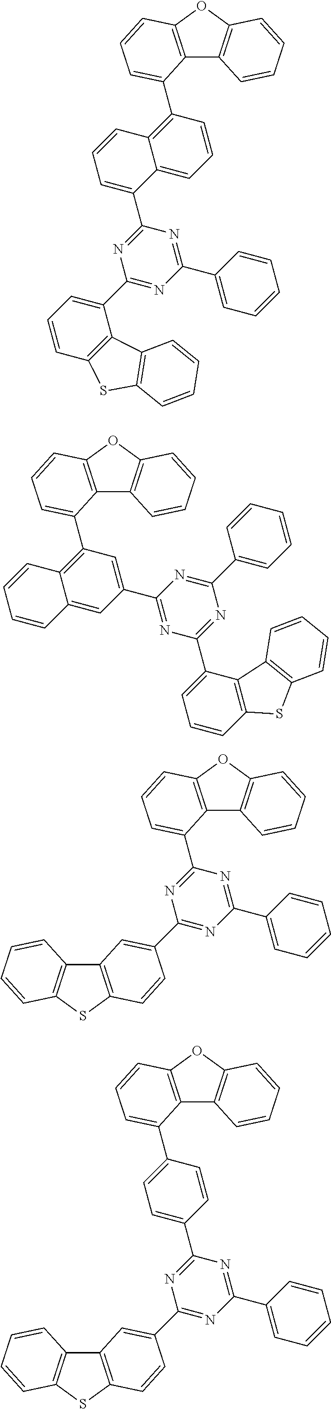

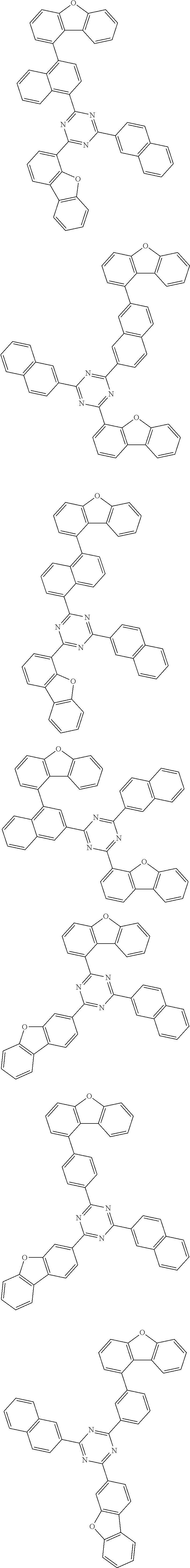

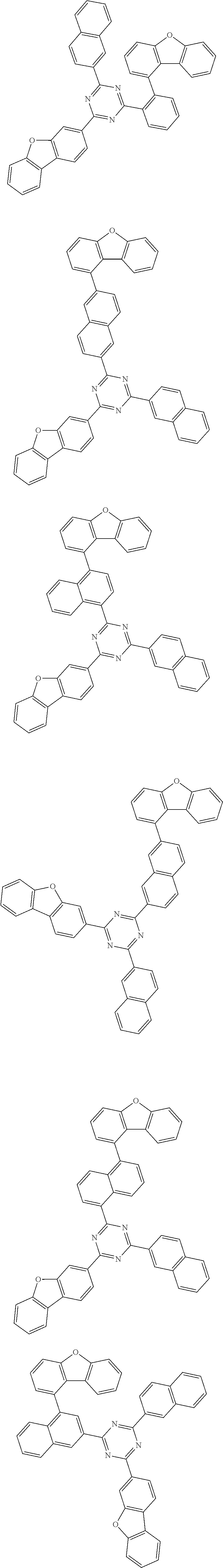

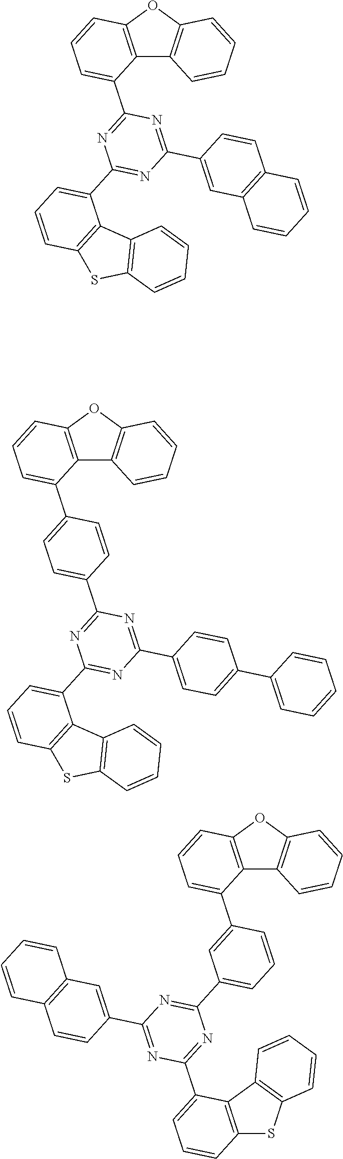 [SMILES: c1ccc2cc(-c3nc(-c4cccc5oc6ccccc6c45)nc(-c4cccc5sc6ccccc6c45)n3)ccc2c1 ; c1ccc(-c2ccc(-c3nc(-c4ccc(-c5cccc6oc7ccccc7c56)cc4)nc(-c4cccc5sc6ccccc6c45)n3)cc2)cc1 ; c1cc(-c2nc(-c3ccc4ccccc4c3)nc(-c3cccc4sc5ccccc5c34)n2)cc(-c2cccc3oc4ccccc4c23)c1]

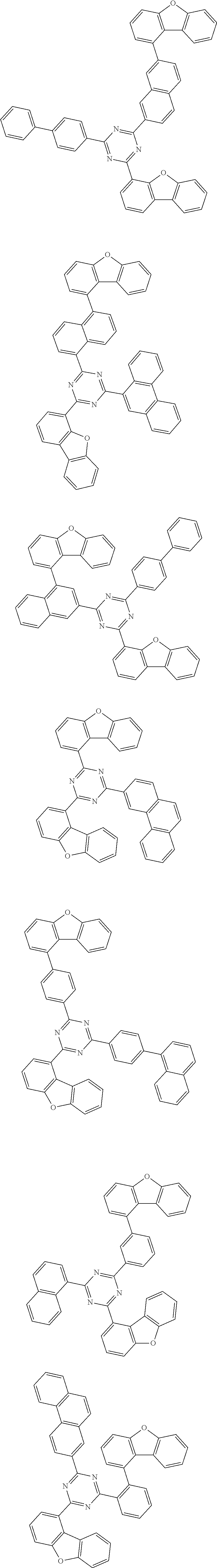

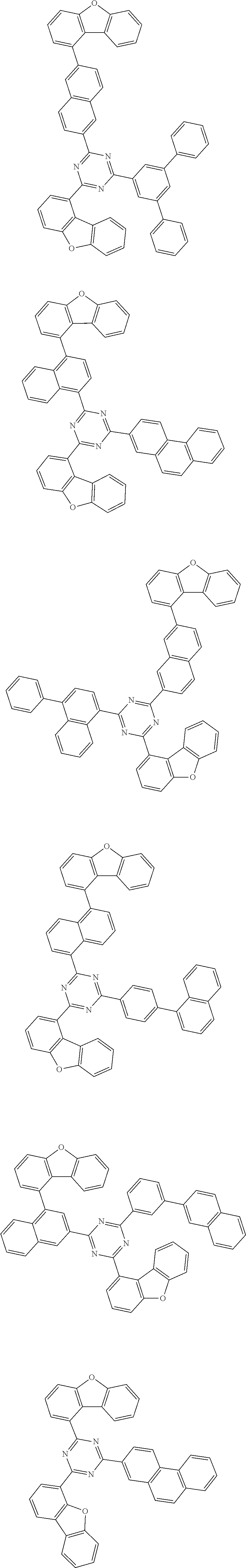

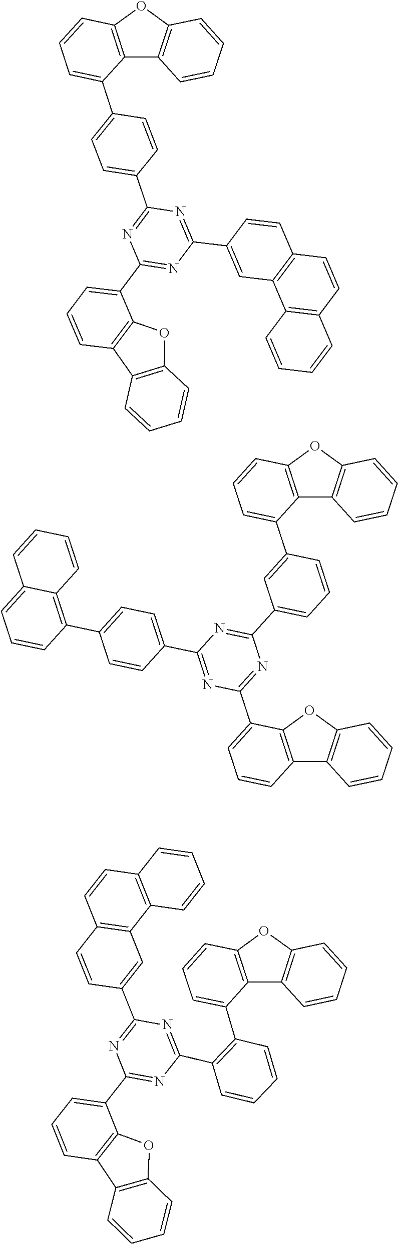 [SMILES: c1ccc2c(c1)ccc1ccc(-c3nc(-c4ccc(-c5cccc6oc7ccccc7c56)cc4)nc(-c4cccc5c4oc4ccccc45)n3)cc12 ; c1cc(-c2nc(-c3ccc(-c4cccc5ccccc45)cc3)nc(-c3cccc4c3oc3ccccc34)n2)cc(-c2cccc3oc4ccccc4c23)c1 ; c1ccc(-c2cccc3oc4ccccc4c23)c(-c2nc(-c3ccc4ccc5ccccc5c4c3)nc(-c3cccc4c3oc3ccccc34)n2)c1]

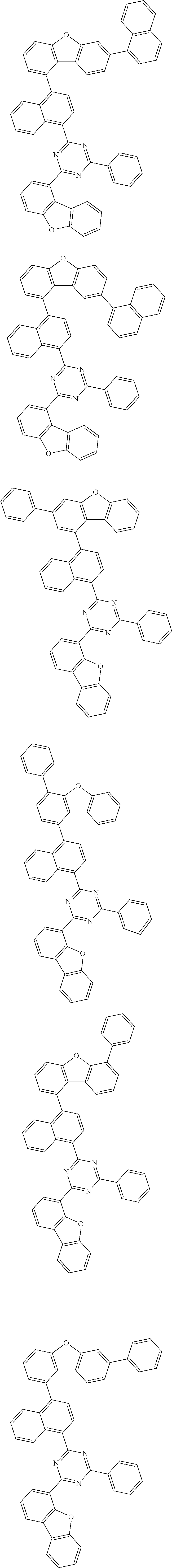

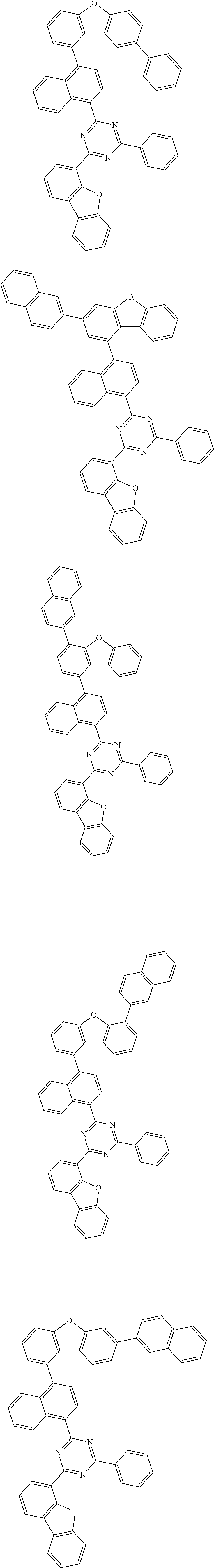 [SMILES: c1ccc(-c2ccc3oc4cccc(-c5ccc(-c6nc(-c7ccccc7)nc(-c7cccc8c7oc7ccccc78)n6)c6ccccc56)c4c3c2)cc1 ; c1ccc(-c2nc(-c3ccc(-c4cc(-c5ccc6ccccc6c5)cc5oc6ccccc6c45)c4ccccc34)nc(-c3cccc4c3oc3ccccc34)n2)cc1 ; c1ccc(-c2nc(-c3ccc(-c4ccc(-c5ccc6ccccc6c5)c5oc6ccccc6c45)c4ccccc34)nc(-c3cccc4c3oc3ccccc34)n2)cc1 ; c1ccc(-c2nc(-c3ccc(-c4cccc5oc6c(-c7ccc8ccccc8c7)cccc6c45)c4ccccc34)nc(-c3cccc4c3oc3ccccc34)n2)cc1 ; c1ccc(-c2nc(-c3ccc(-c4cccc5oc6cc(-c7ccc8ccccc8c7)ccc6c45)c4ccccc34)nc(-c3cccc4c3oc3ccccc34)n2)cc1]

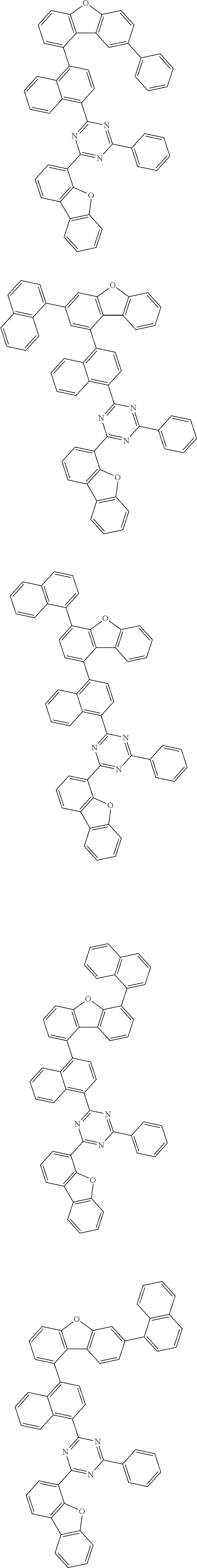 [SMILES: c1ccc(-c2ccc3oc4cccc(-c5ccc(-c6nc(-c7ccccc7)nc(-c7cccc8c7oc7ccccc78)n6)c6ccccc56)c4c3c2)cc1 ; c1ccc(-c2nc(-c3ccc(-c4cc(-c5cccc6ccccc56)cc5oc6ccccc6c45)c4ccccc34)nc(-c3cccc4c3oc3ccccc34)n2)cc1 ; c1ccc(-c2nc(-c3ccc(-c4ccc(-c5cccc6ccccc56)c5oc6ccccc6c45)c4ccccc34)nc(-c3cccc4c3oc3ccccc34)n2)cc1 ; c1ccc(-c2nc(-c3ccc(-c4cccc5oc6c(-c7cccc8ccccc78)cccc6c45)c4ccccc34)nc(-c3cccc4c3oc3ccccc34)n2)cc1 ; c1ccc(-c2nc(-c3ccc(-c4cccc5oc6cc(-c7cccc8ccccc78)ccc6c45)c4ccccc34)nc(-c3cccc4c3oc3ccccc34)n2)cc1]

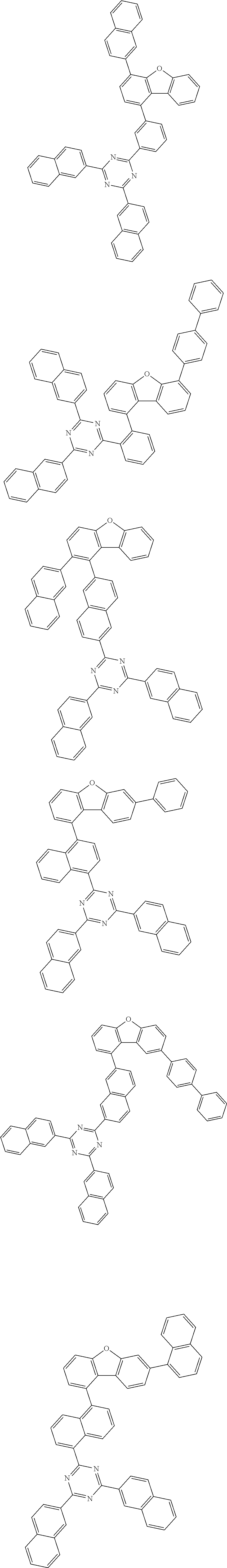

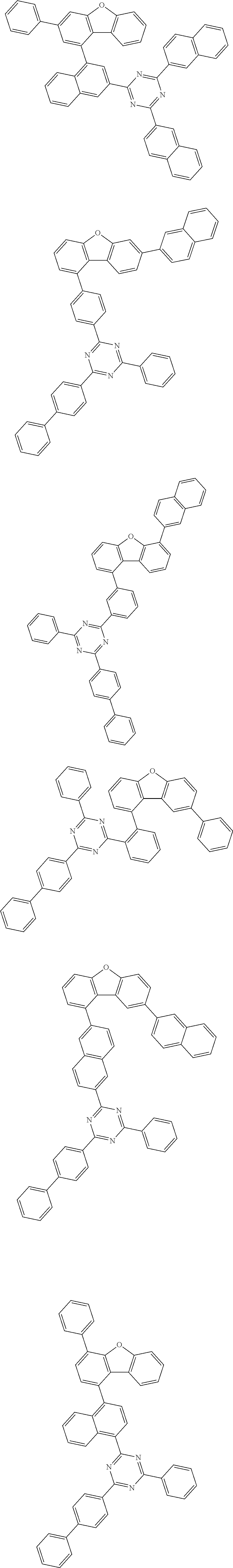

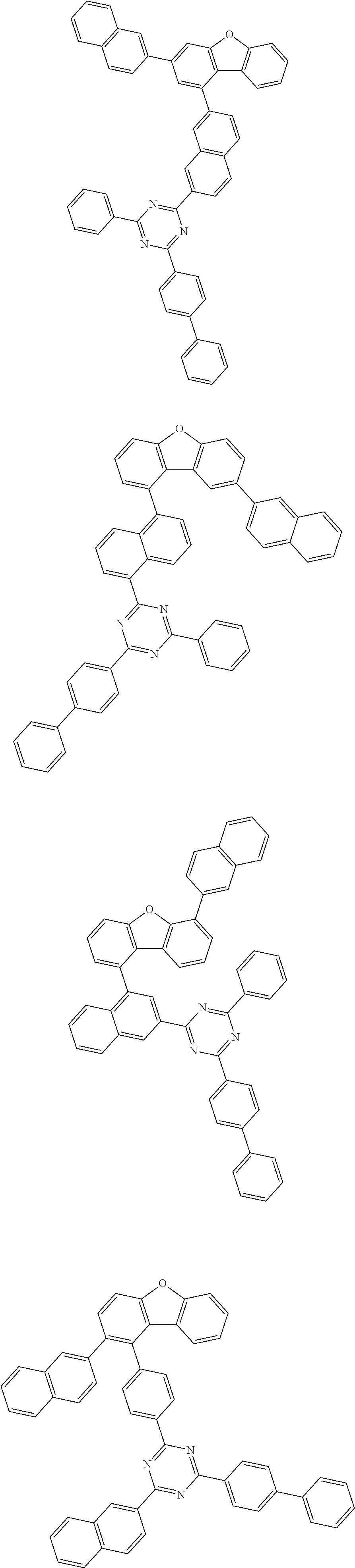 [SMILES: c1ccc(-c2ccc(-c3nc(-c4ccccc4)nc(-c4ccc5ccc(-c6cc(-c7ccc8ccccc8c7)cc7oc8ccccc8c67)cc5c4)n3)cc2)cc1 ; c1ccc(-c2ccc(-c3nc(-c4ccccc4)nc(-c4cccc5c(-c6cccc7oc8ccc(-c9ccc%10ccccc%10c9)cc8c67)cccc45)n3)cc2)cc1 ; c1ccc(-c2ccc(-c3nc(-c4ccccc4)nc(-c4cc(-c5cccc6oc7c(-c8ccc9ccccc9c8)cccc7c56)c5ccccc5c4)n3)cc2)cc1 ; c1ccc(-c2ccc(-c3nc(-c4ccc(-c5c(-c6ccc7ccccc7c6)ccc6oc7ccccc7c56)cc4)nc(-c4ccc5ccccc5c4)n3)cc2)cc1]

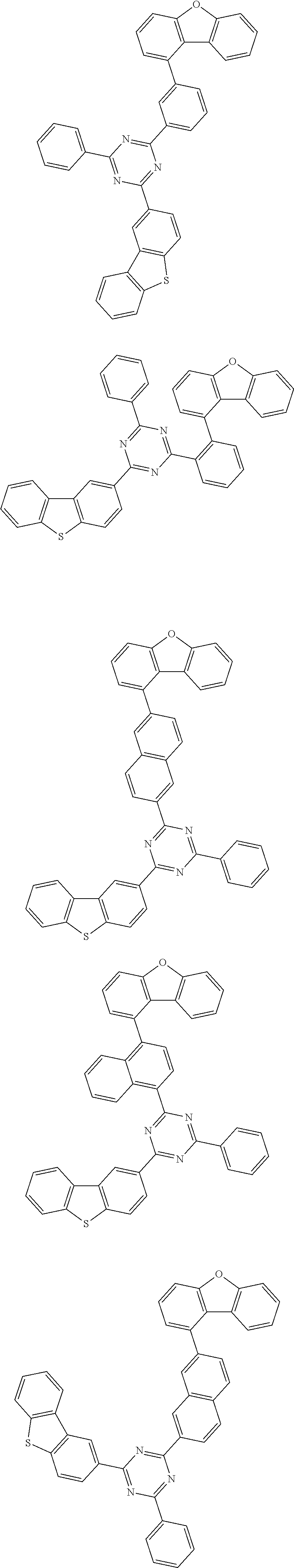 [SMILES: c1ccc(-c2nc(-c3cccc(-c4cccc5oc6ccccc6c45)c3)nc(-c3ccc4sc5ccccc5c4c3)n2)cc1 ; c1ccc(-c2nc(-c3ccc4sc5ccccc5c4c3)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4sc5ccccc5c4c3)n2)cc1 ; c1ccc(-c2nc(-c3ccc4sc5ccccc5c4c3)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4sc5ccccc5c4c3)n2)cc1]

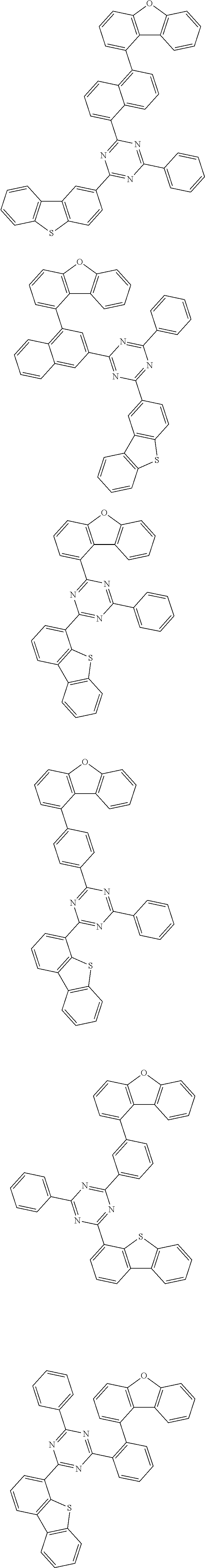

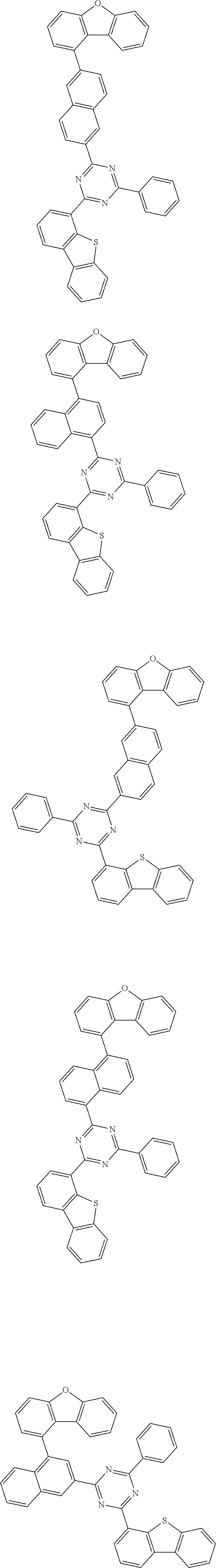 [SMILES: c1ccc(-c2nc(-c3ccc4cc(-c5cccc6oc7ccccc7c56)ccc4c3)nc(-c3cccc4c3sc3ccccc34)n2)cc1 ; c1ccc(-c2nc(-c3ccc(-c4cccc5oc6ccccc6c45)c4ccccc34)nc(-c3cccc4c3sc3ccccc34)n2)cc1 ; c1ccc(-c2nc(-c3ccc4ccc(-c5cccc6oc7ccccc7c56)cc4c3)nc(-c3cccc4c3sc3ccccc34)n2)cc1 ; c1ccc(-c2nc(-c3cccc4c(-c5cccc6oc7ccccc7c56)cccc34)nc(-c3cccc4c3sc3ccccc34)n2)cc1 ; c1ccc(-c2nc(-c3cc(-c4cccc5oc6ccccc6c45)c4ccccc4c3)nc(-c3cccc4c3sc3ccccc34)n2)cc1]

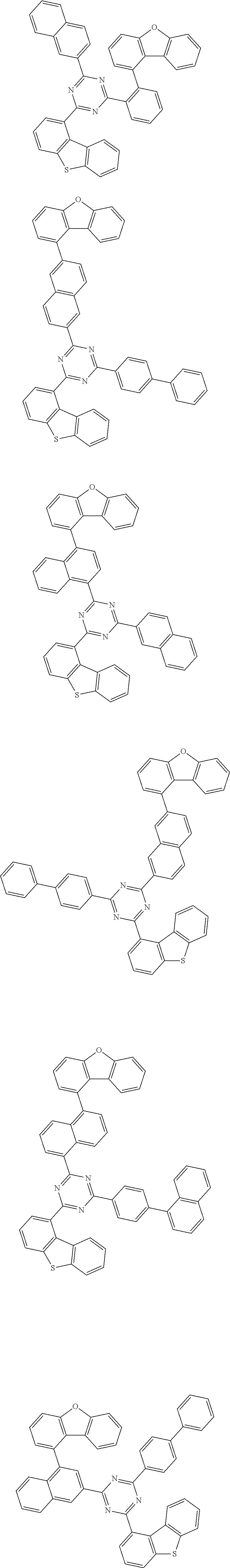

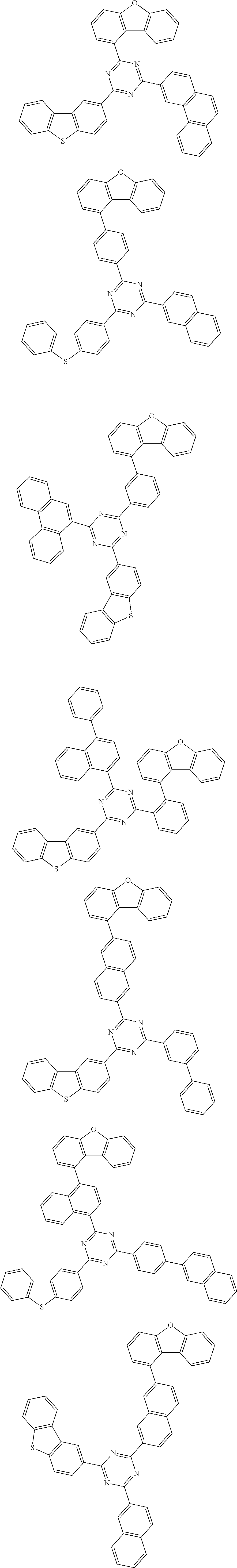

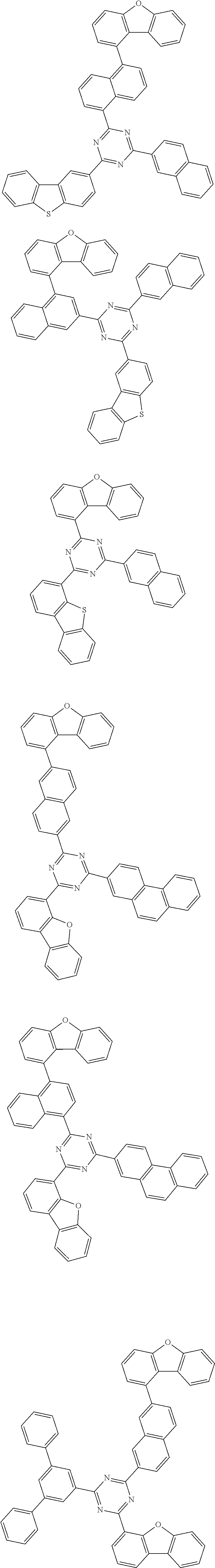

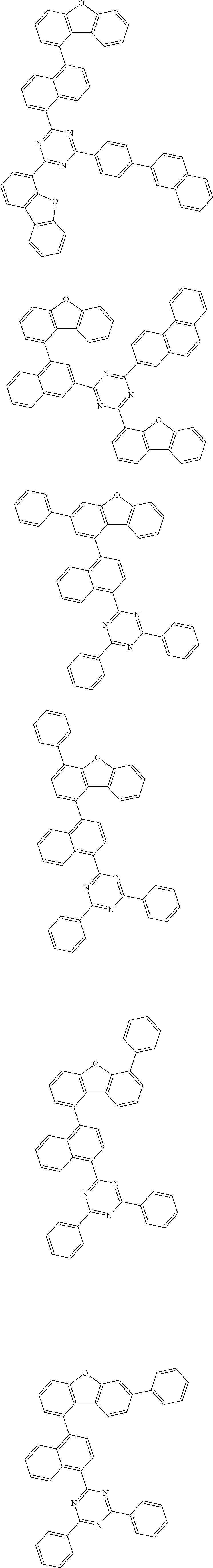

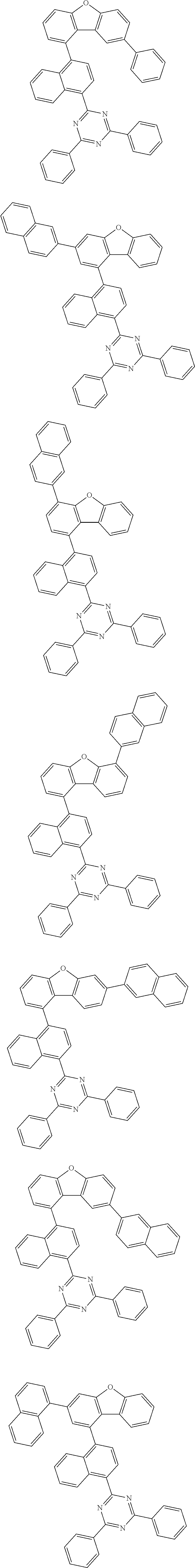

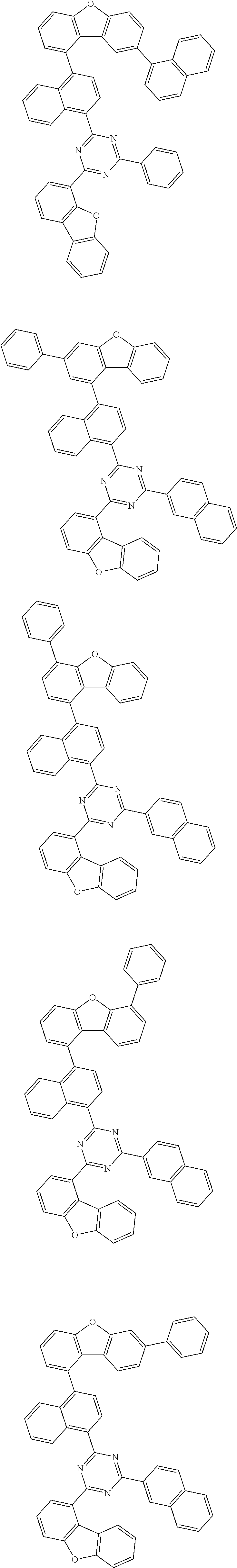

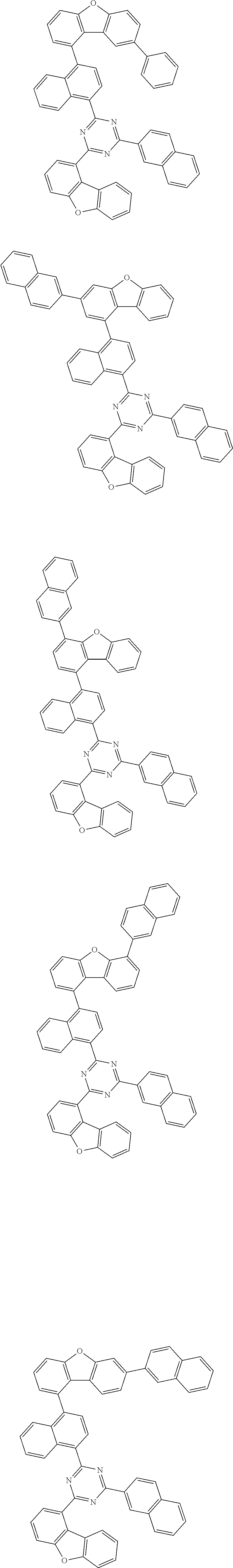

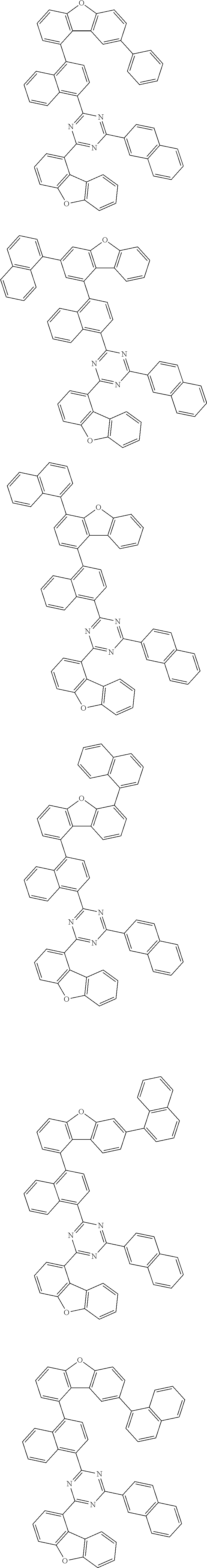

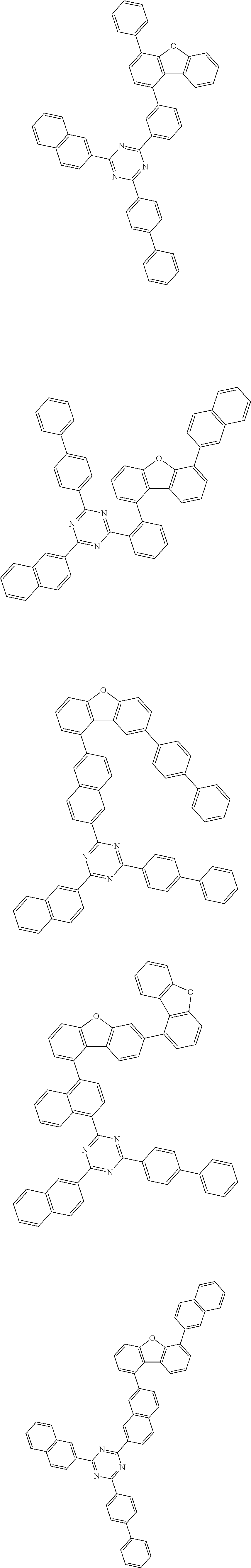

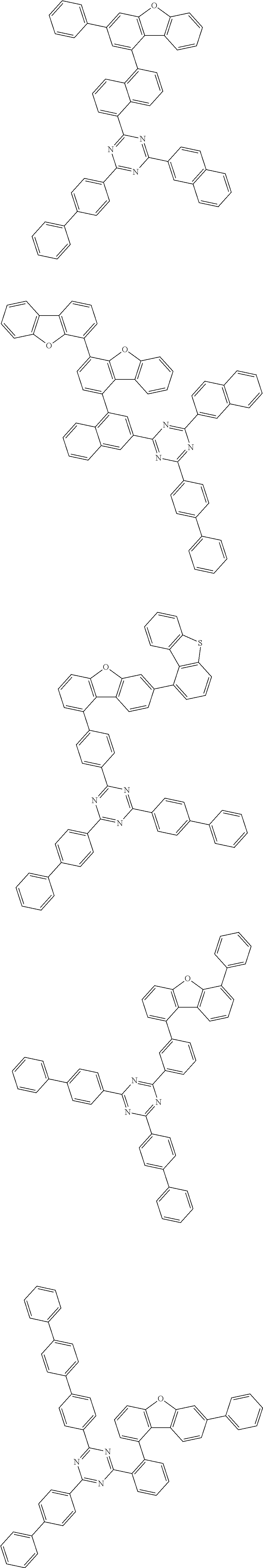

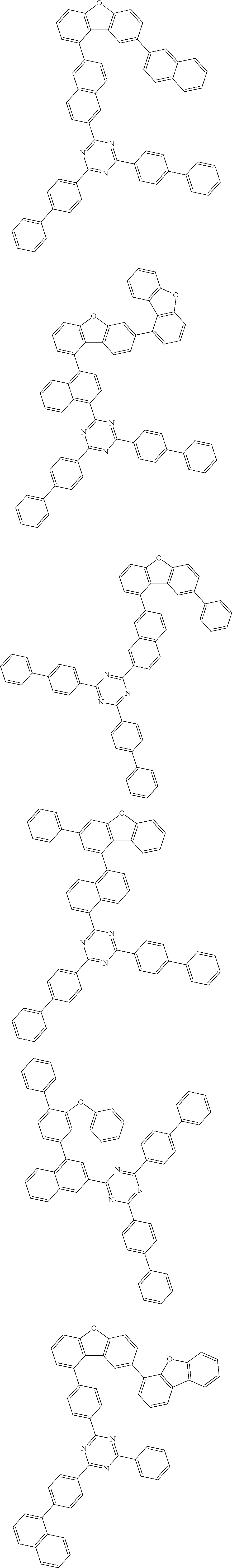

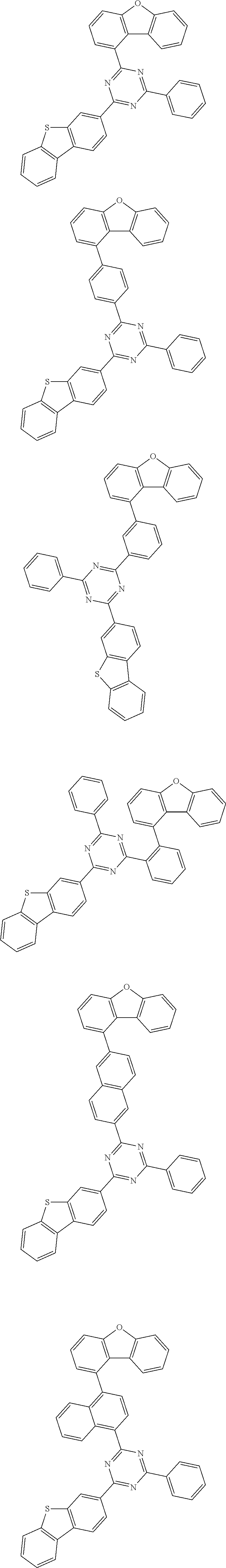 [SMILES: c1ccc(-c2nc(-c3ccc4c(c3)sc3ccccc34)nc(-c3cccc4oc5ccccc5c34)n2)cc1 ; c1ccc(-c2nc(-c3ccc(-c4cccc5oc6ccccc6c45)cc3)nc(-c3ccc4c(c3)sc3ccccc34)n2)cc1 ; c1ccc(-c2nc(-c3cccc(-c4cccc5oc6ccccc6c45)c3)nc(-c3ccc4c(c3)sc3ccccc34)n2)cc1 ; c1ccc(-c2nc(-c3ccc4c(c3)sc3ccccc34)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4c(c3)sc3ccccc34)n2)cc1 ; c1ccc(-c2nc(-c3ccc4c(c3)sc3ccccc34)nc(-c3ccc(-c4cccc5oc6ccccc6c45)c4ccccc34)n2)cc1]

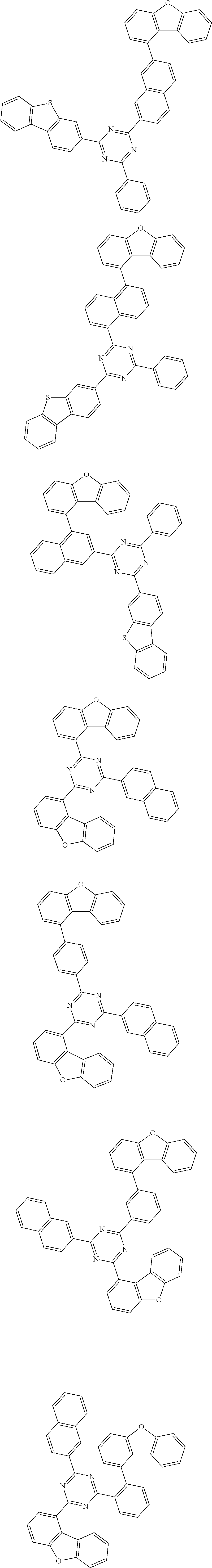

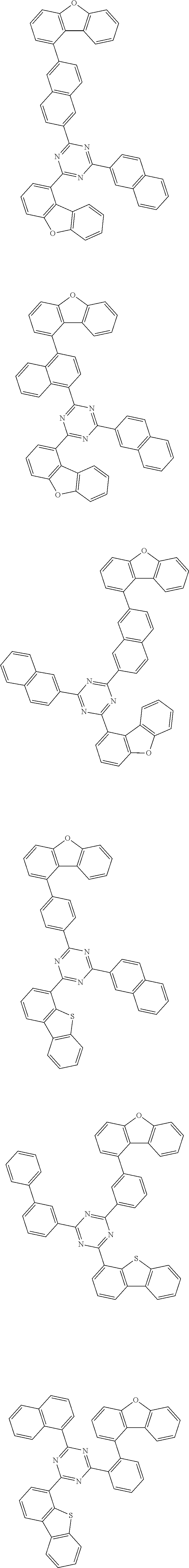

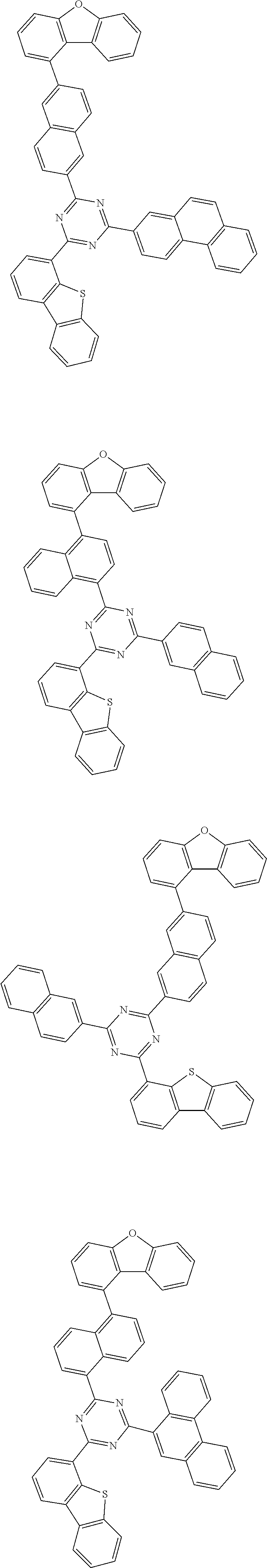 [SMILES: c1ccc2c(c1)ccc1cc(-c3nc(-c4ccc5cc(-c6cccc7oc8ccccc8c67)ccc5c4)nc(-c4cccc5c4sc4ccccc45)n3)ccc12 ; c1ccc2cc(-c3nc(-c4ccc(-c5cccc6oc7ccccc7c56)c5ccccc45)nc(-c4cccc5c4sc4ccccc45)n3)ccc2c1 ; c1ccc2cc(-c3nc(-c4ccc5ccc(-c6cccc7oc8ccccc8c67)cc5c4)nc(-c4cccc5c4sc4ccccc45)n3)ccc2c1 ; c1ccc2c(c1)cc(-c1nc(-c3cccc4c(-c5cccc6oc7ccccc7c56)cccc34)nc(-c3cccc4c3sc3ccccc34)n1)c1ccccc12]

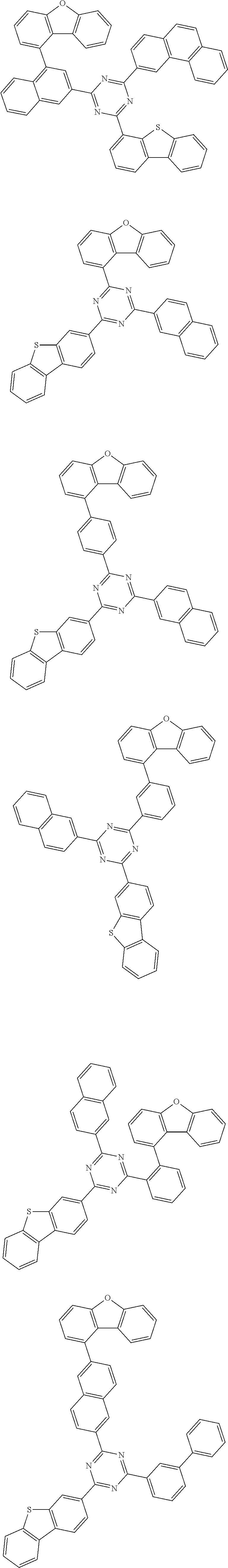

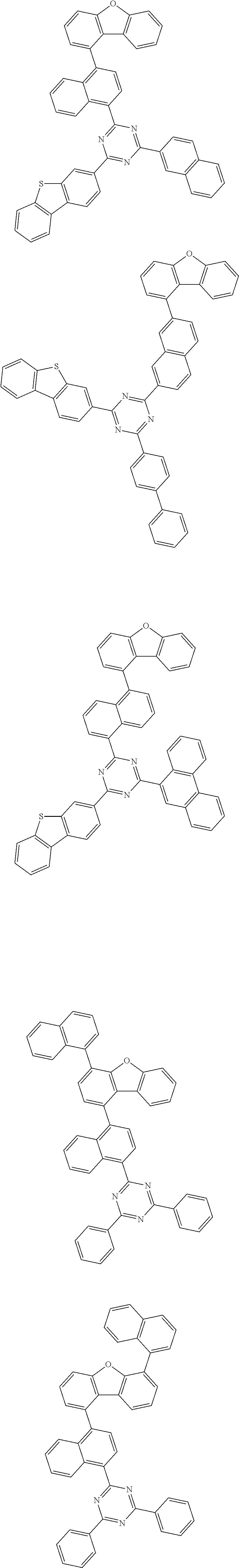 [SMILES: c1ccc2cc(-c3nc(-c4ccc5c(c4)sc4ccccc45)nc(-c4ccc(-c5cccc6oc7ccccc7c56)c5ccccc45)n3)ccc2c1 ; c1ccc(-c2ccc(-c3nc(-c4ccc5ccc(-c6cccc7oc8ccccc8c67)cc5c4)nc(-c4ccc5c(c4)sc4ccccc45)n3)cc2)cc1 ; c1ccc2c(c1)cc(-c1nc(-c3ccc4c(c3)sc3ccccc34)nc(-c3cccc4c(-c5cccc6oc7ccccc7c56)cccc34)n1)c1ccccc12 ; c1ccc(-c2nc(-c3ccccc3)nc(-c3ccc(-c4ccc(-c5cccc6ccccc56)c5oc6ccccc6c45)c4ccccc34)n2)cc1 ; c1ccc(-c2nc(-c3ccccc3)nc(-c3ccc(-c4cccc5oc6c(-c7cccc8ccccc78)cccc6c45)c4ccccc34)n2)cc1]

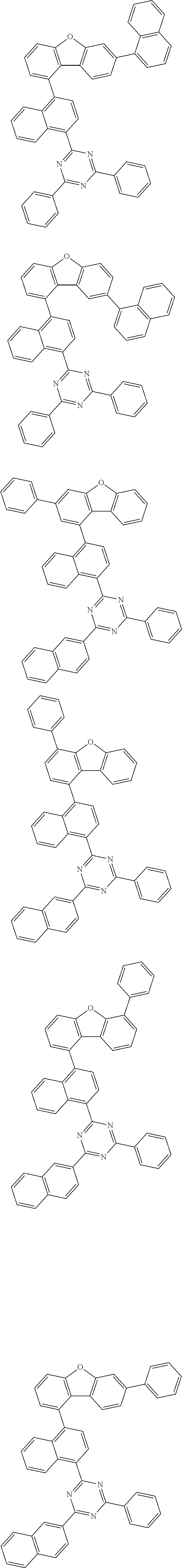

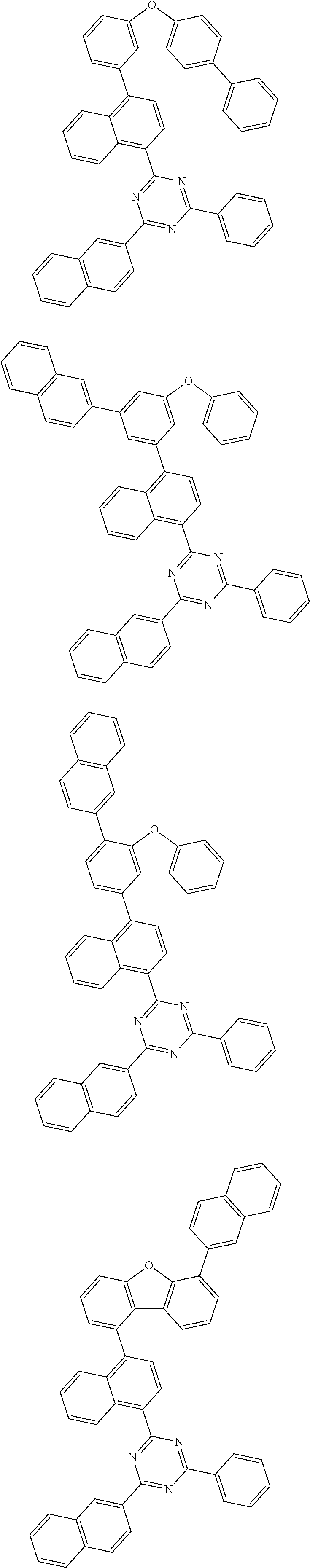

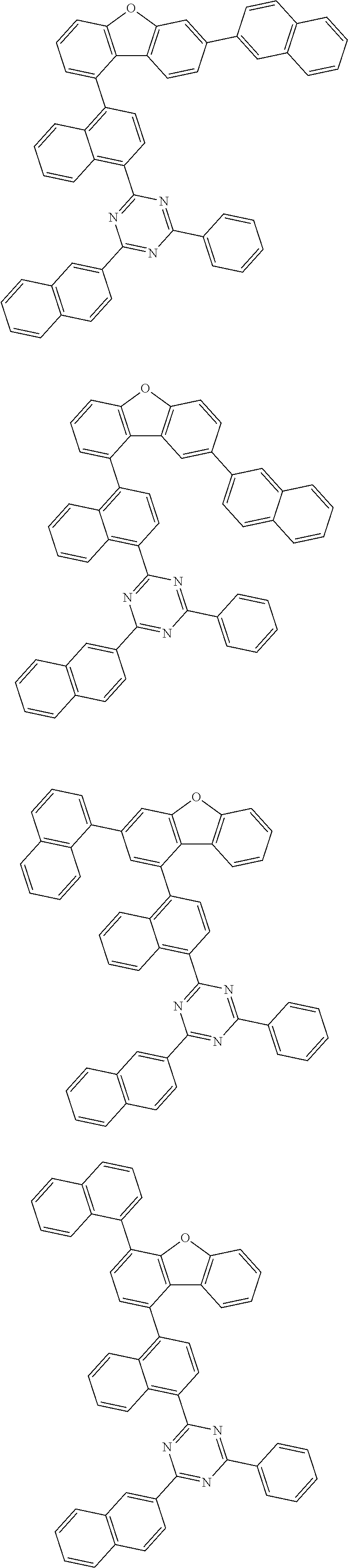

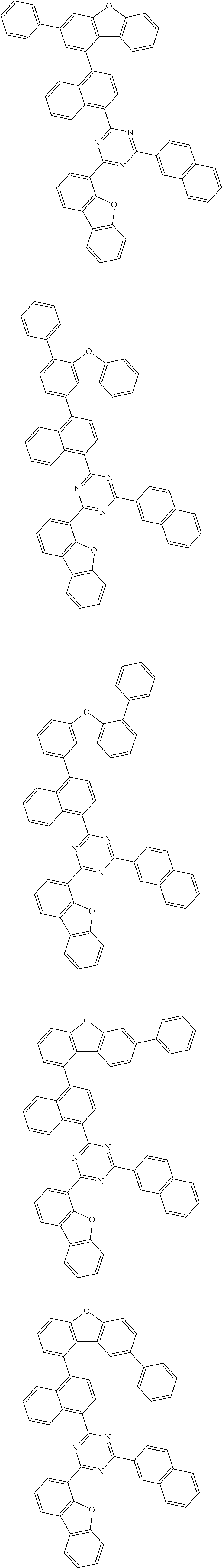

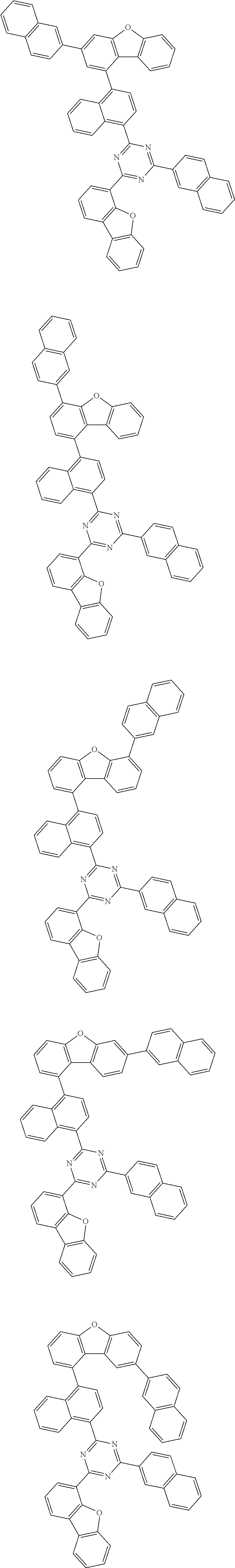

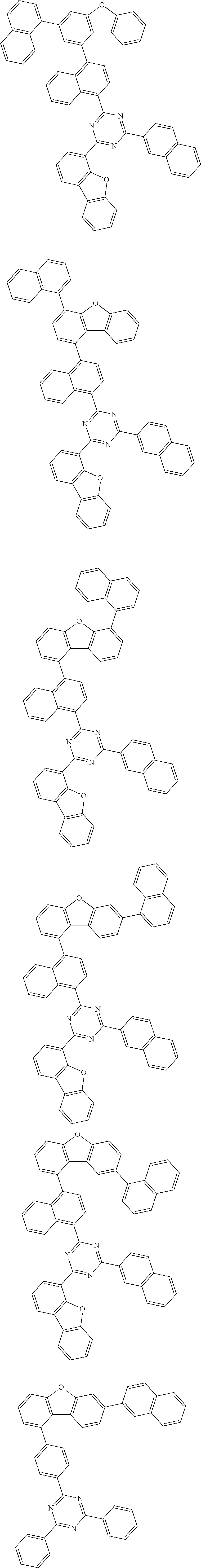

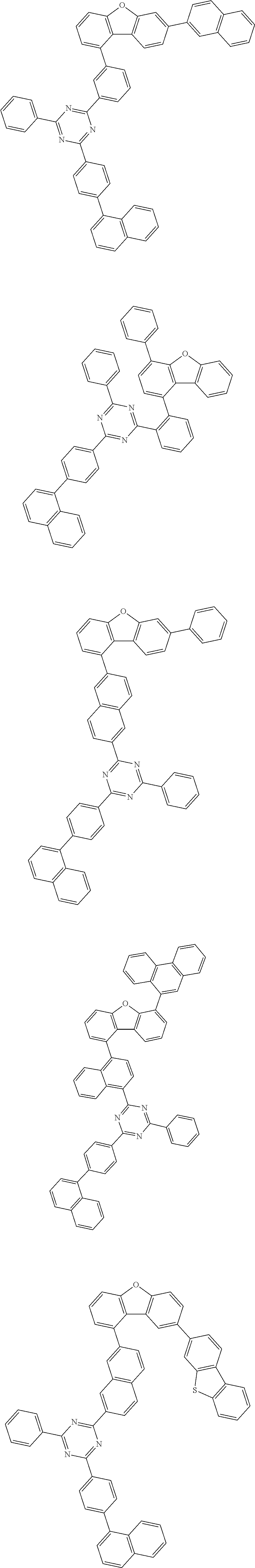

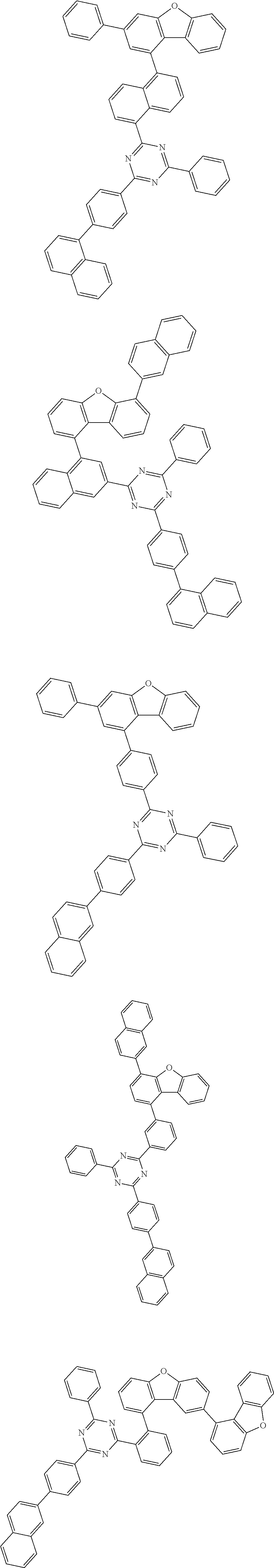

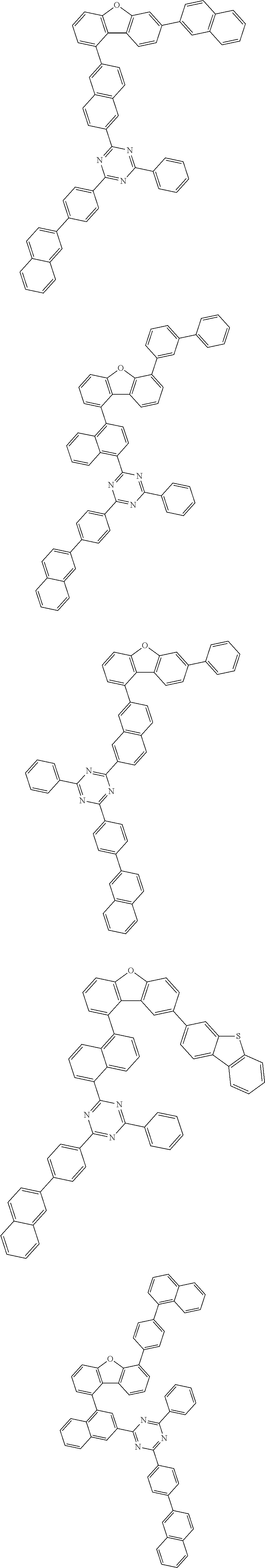

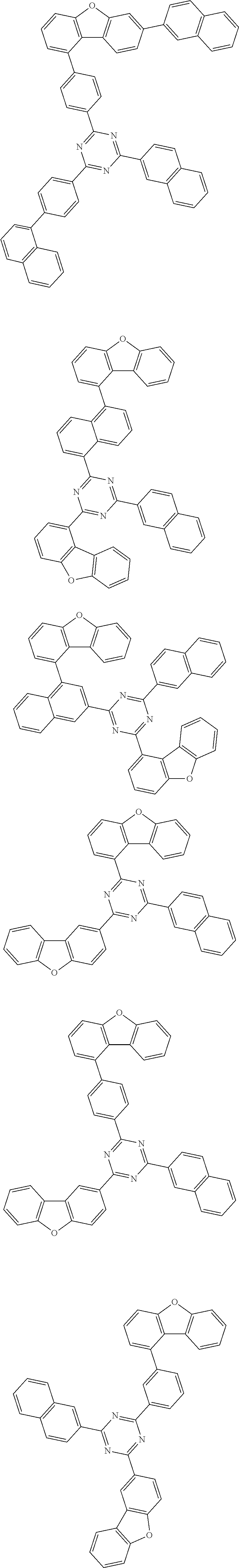

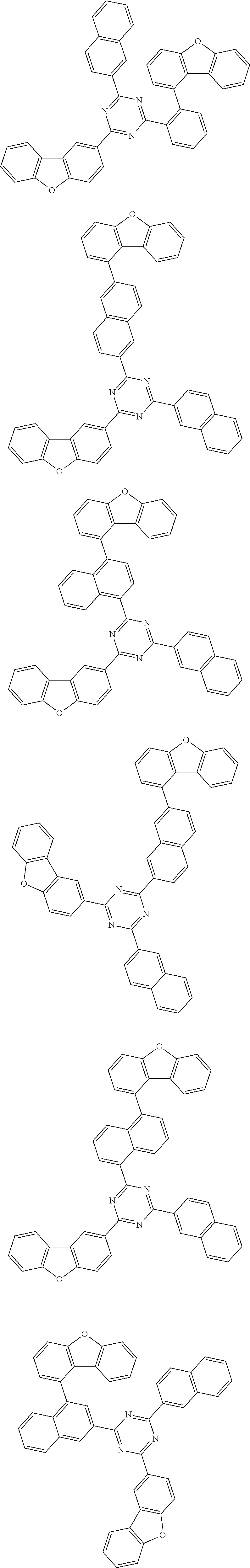

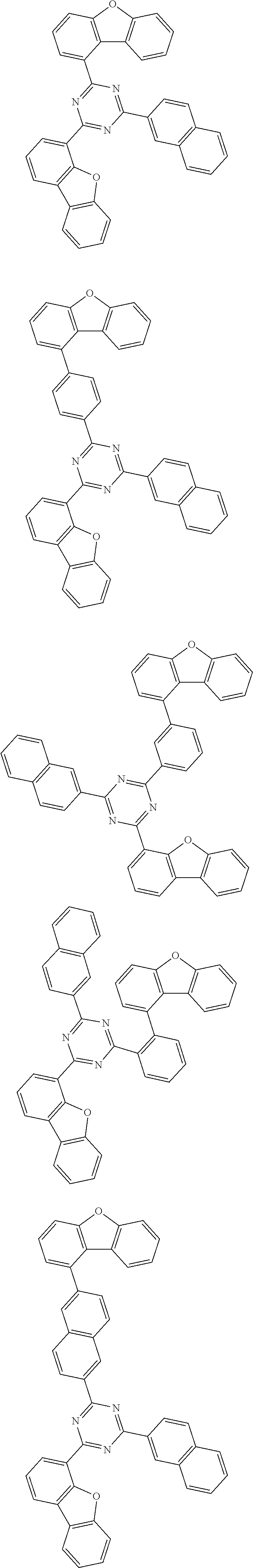

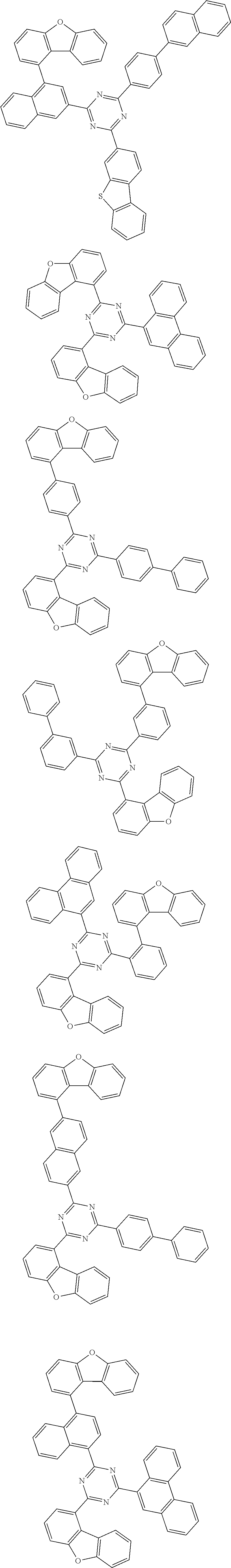

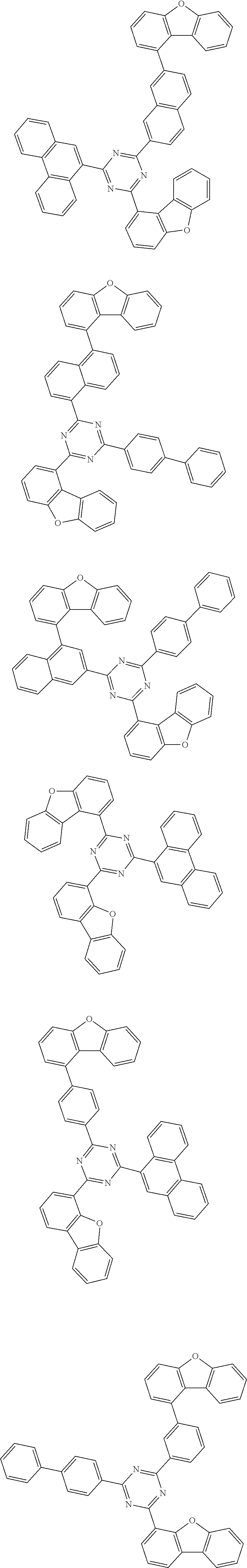

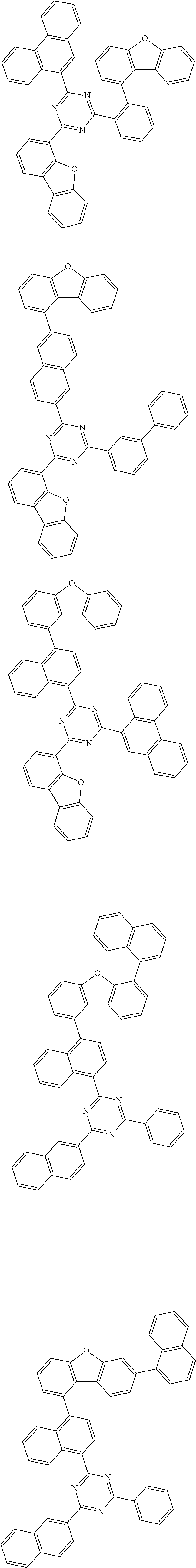 [SMILES: c1ccc(-c2cccc3oc4ccccc4c23)c(-c2nc(-c3cc4ccccc4c4ccccc34)nc(-c3cccc4c3oc3ccccc34)n2)c1 ; c1ccc(-c2cccc(-c3nc(-c4ccc5cc(-c6cccc7oc8ccccc8c67)ccc5c4)nc(-c4cccc5c4oc4ccccc45)n3)c2)cc1 ; c1ccc2c(c1)cc(-c1nc(-c3ccc(-c4cccc5oc6ccccc6c45)c4ccccc34)nc(-c3cccc4c3oc3ccccc34)n1)c1ccccc12 ; c1ccc(-c2nc(-c3ccc4ccccc4c3)nc(-c3ccc(-c4cccc5oc6c(-c7cccc8ccccc78)cccc6c45)c4ccccc34)n2)cc1 ; c1ccc(-c2nc(-c3ccc4ccccc4c3)nc(-c3ccc(-c4cccc5oc6cc(-c7cccc8ccccc78)ccc6c45)c4ccccc34)n2)cc1]

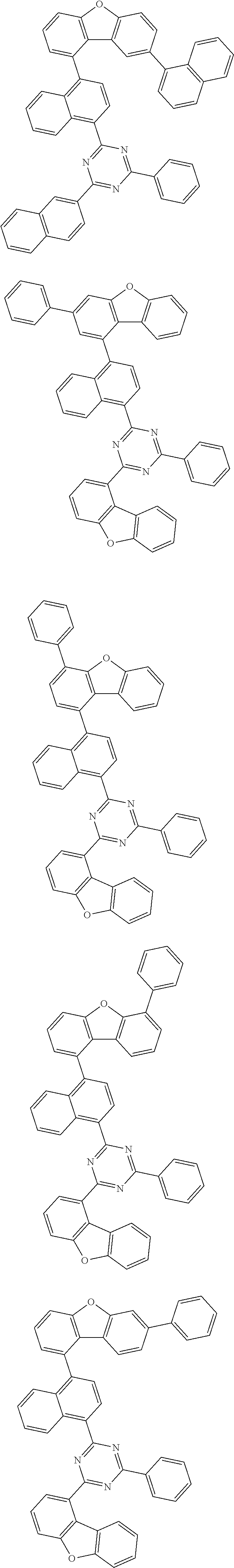

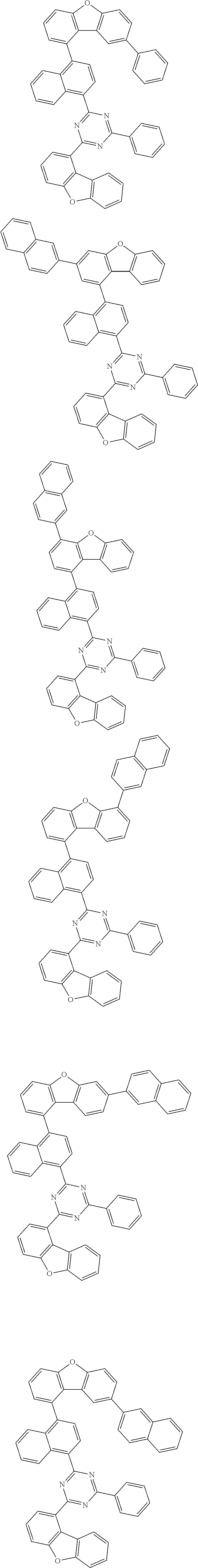

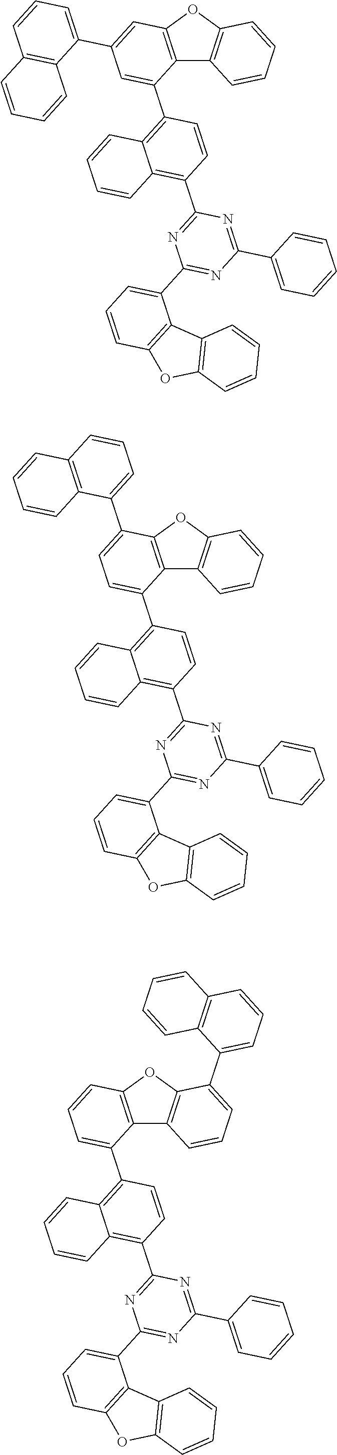 [SMILES: c1ccc(-c2nc(-c3ccc(-c4cc(-c5cccc6ccccc56)cc5oc6ccccc6c45)c4ccccc34)nc(-c3cccc4oc5ccccc5c34)n2)cc1 ; c1ccc(-c2nc(-c3ccc(-c4ccc(-c5cccc6ccccc56)c5oc6ccccc6c45)c4ccccc34)nc(-c3cccc4oc5ccccc5c34)n2)cc1 ; c1ccc(-c2nc(-c3ccc(-c4cccc5oc6c(-c7cccc8ccccc78)cccc6c45)c4ccccc34)nc(-c3cccc4oc5ccccc5c34)n2)cc1]

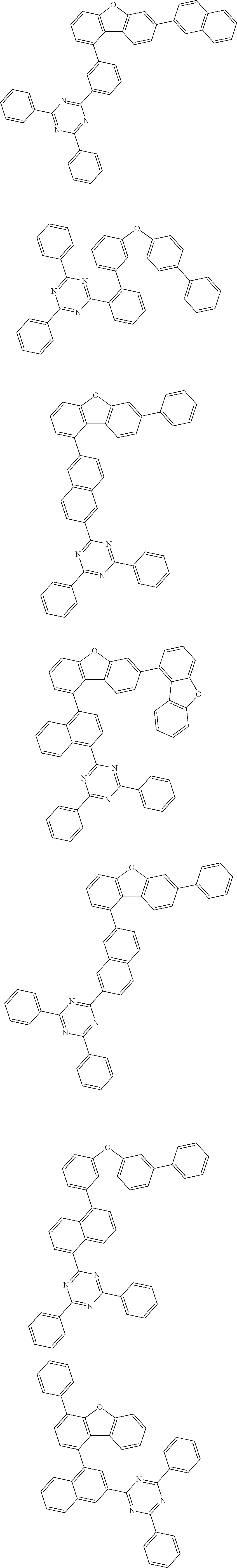

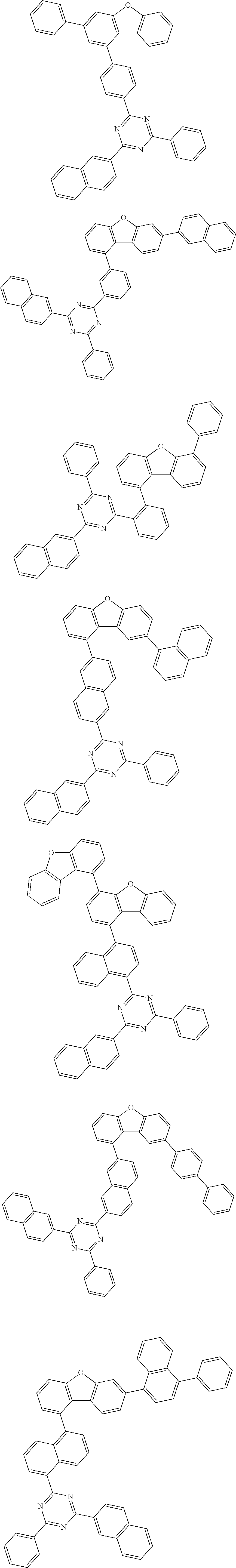

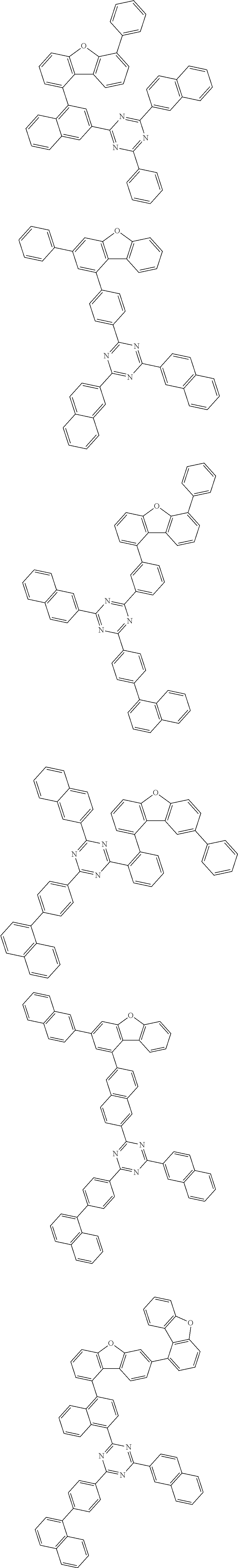

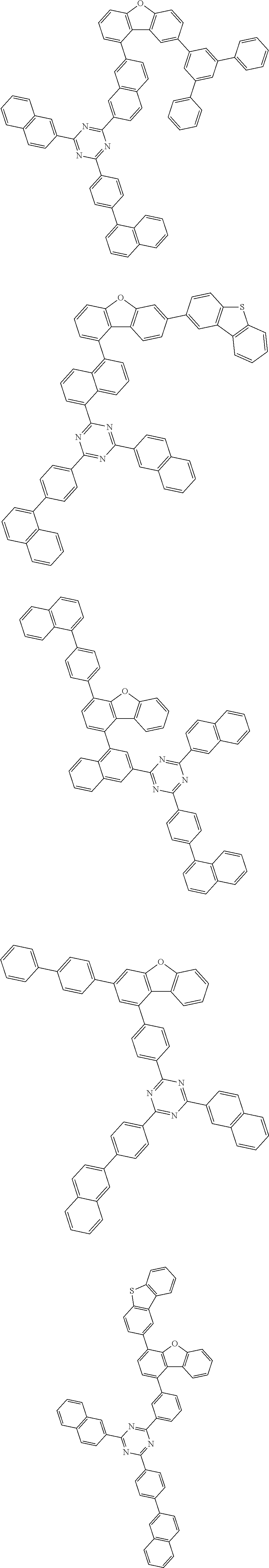

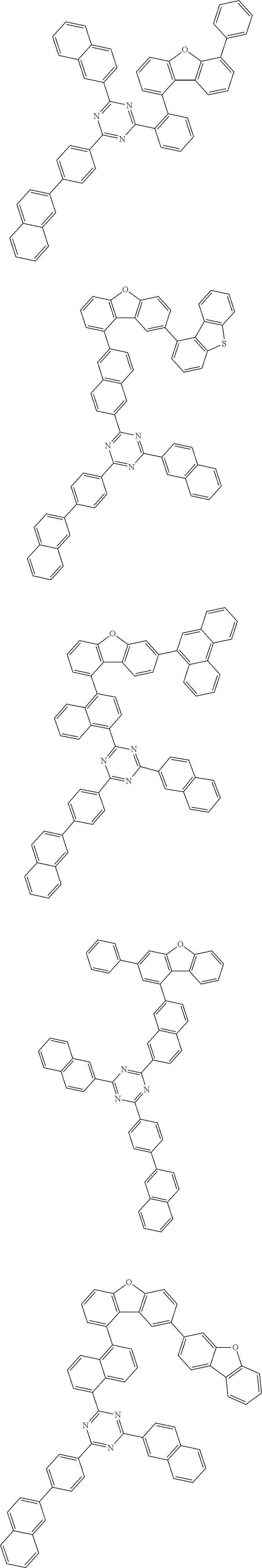

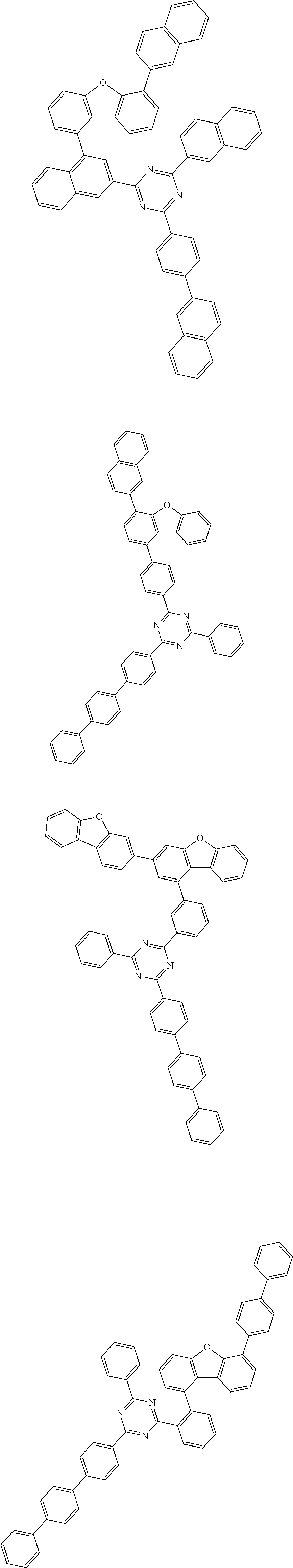 [SMILES: c1ccc2cc(-c3ccc(-c4nc(-c5ccc6ccccc6c5)nc(-c5cc(-c6cccc7oc8c(-c9ccc%10ccccc%10c9)cccc8c67)c6ccccc6c5)n4)cc3)ccc2c1 ; c1ccc(-c2ccc(-c3ccc(-c4nc(-c5ccccc5)nc(-c5ccc(-c6ccc(-c7ccc8ccccc8c7)c7oc8ccccc8c67)cc5)n4)cc3)cc2)cc1 ; c1ccc(-c2ccc(-c3ccc(-c4nc(-c5ccccc5)nc(-c5cccc(-c6cc(-c7ccc8c(c7)oc7ccccc78)cc7oc8ccccc8c67)c5)n4)cc3)cc2)cc1 ; c1ccc(-c2ccc(-c3ccc(-c4nc(-c5ccccc5)nc(-c5ccccc5-c5cccc6oc7c(-c8ccc(-c9ccccc9)cc8)cccc7c56)n4)cc3)cc2)cc1]

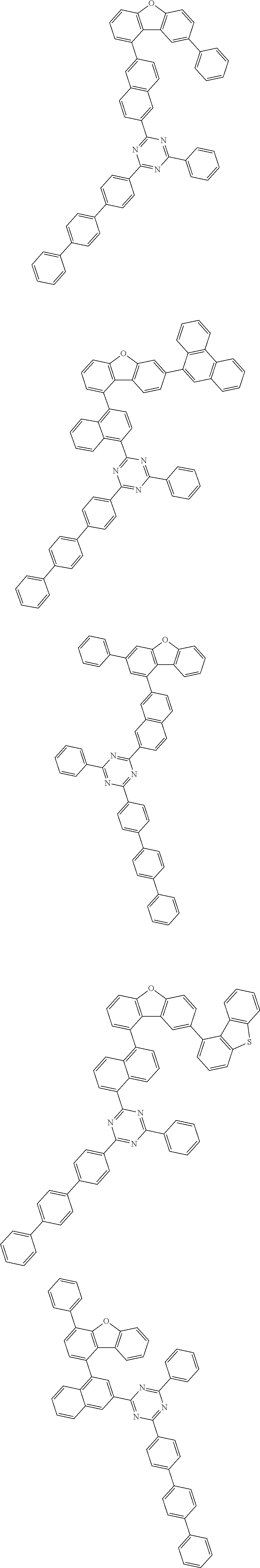

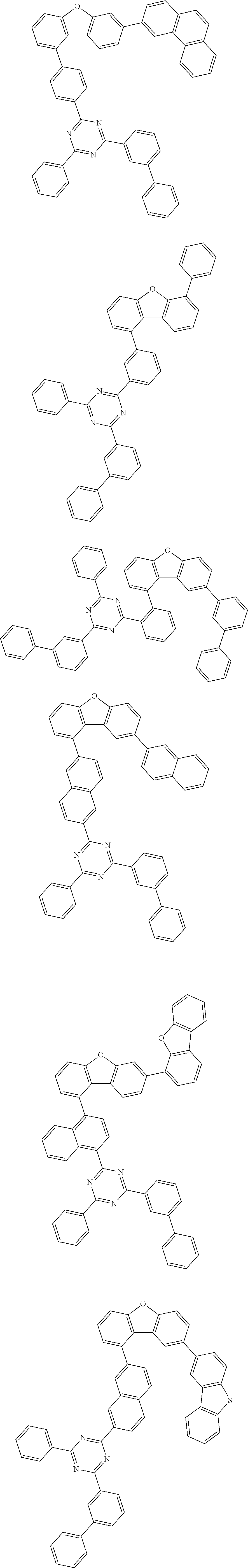

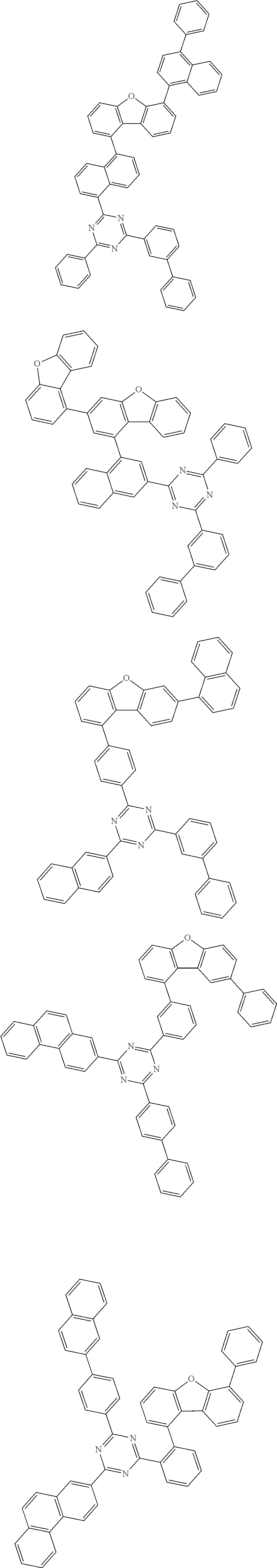

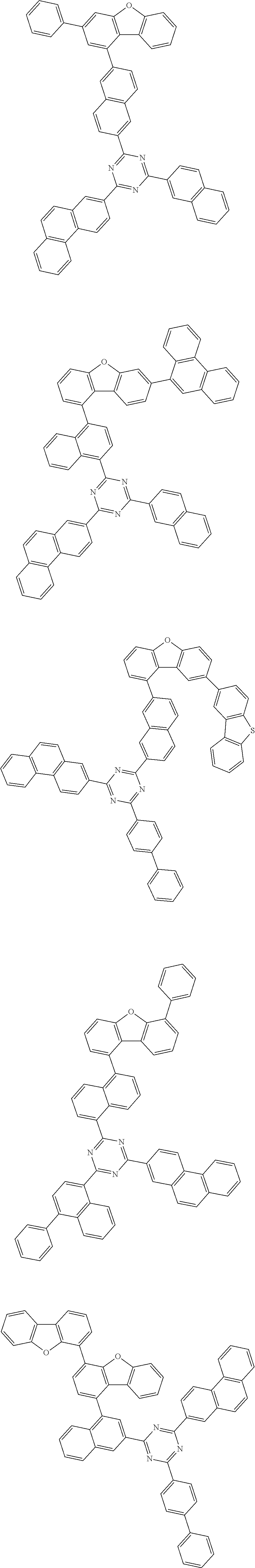

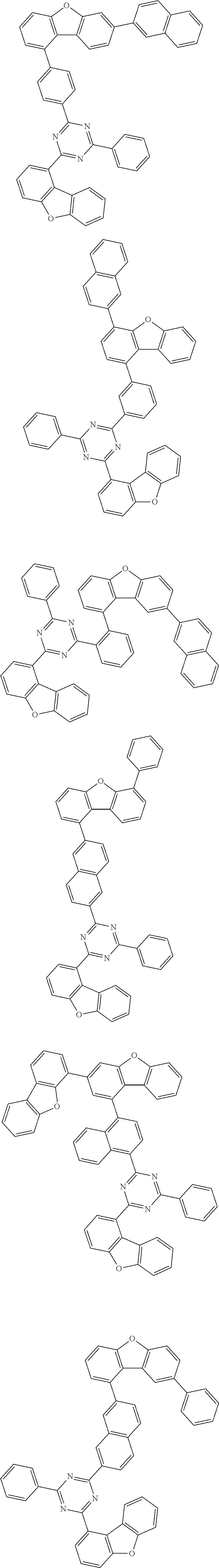

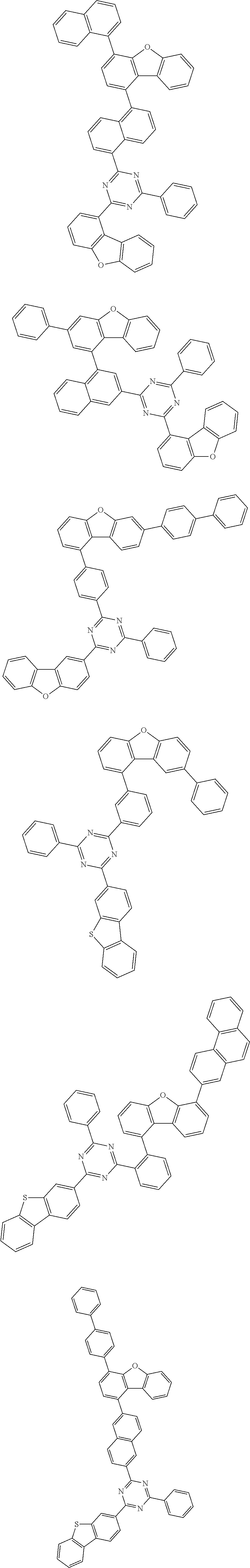

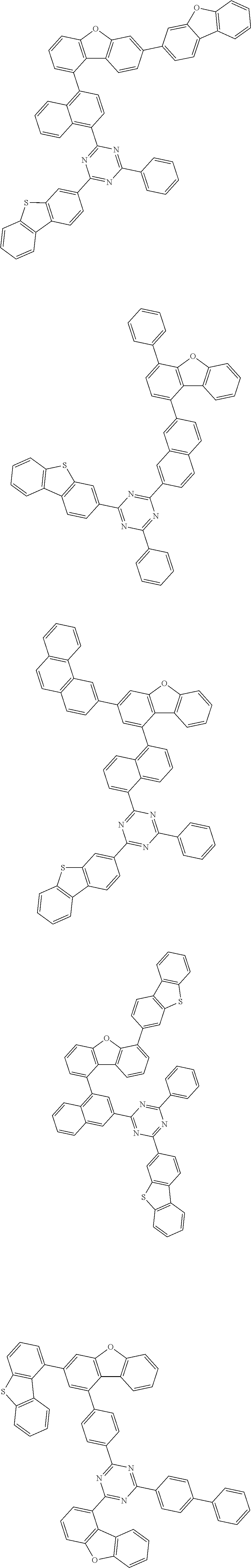

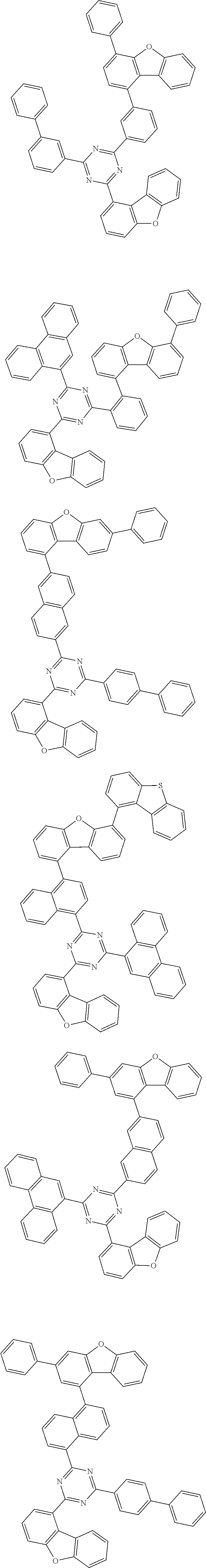

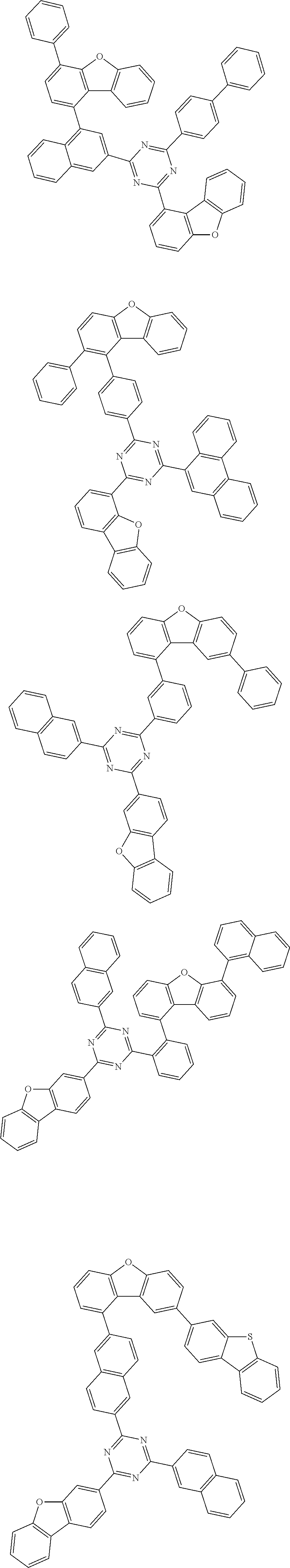

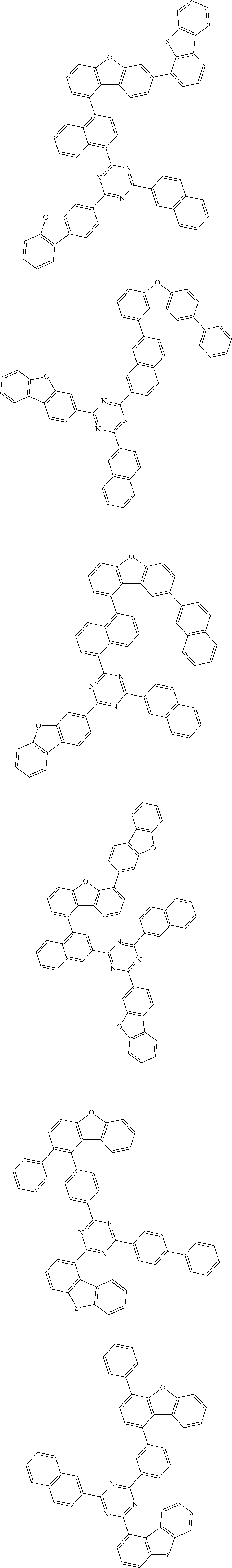

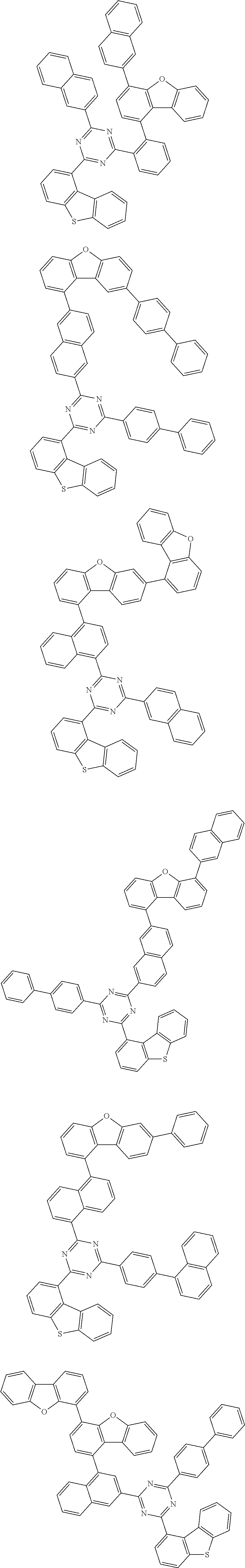

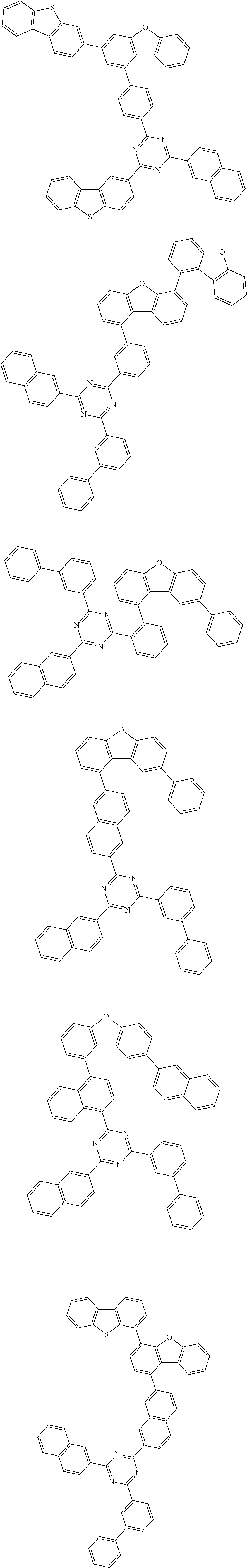

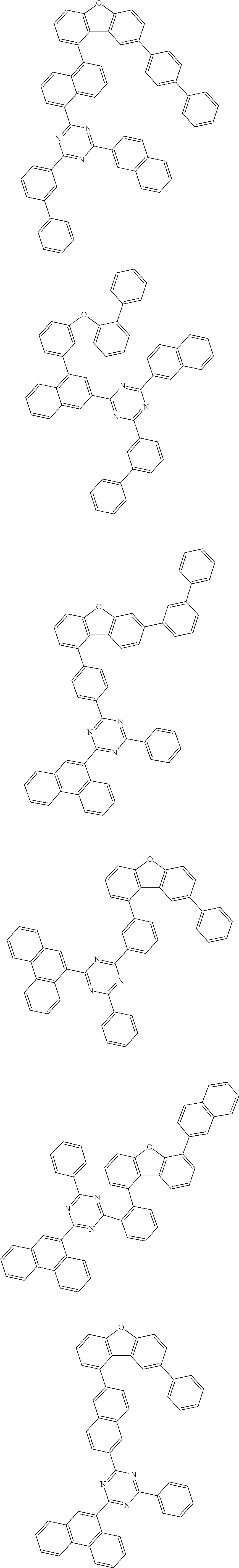

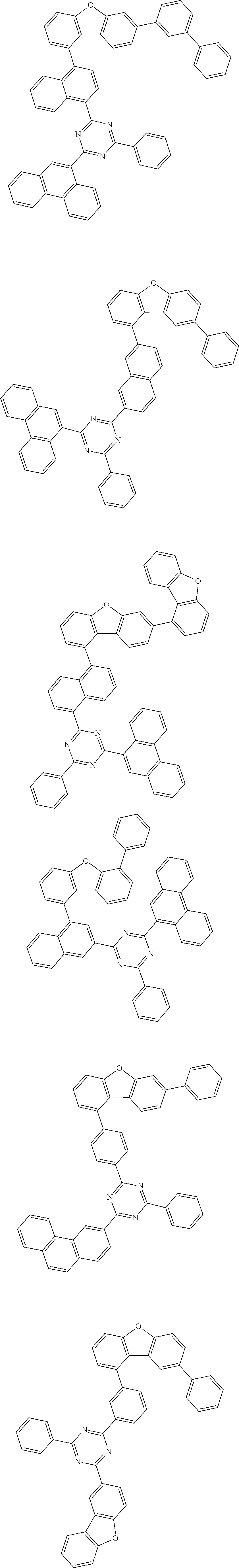

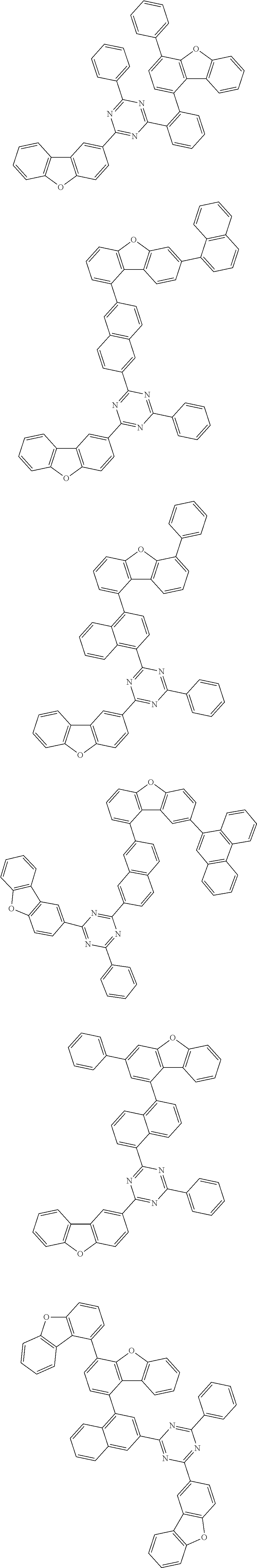

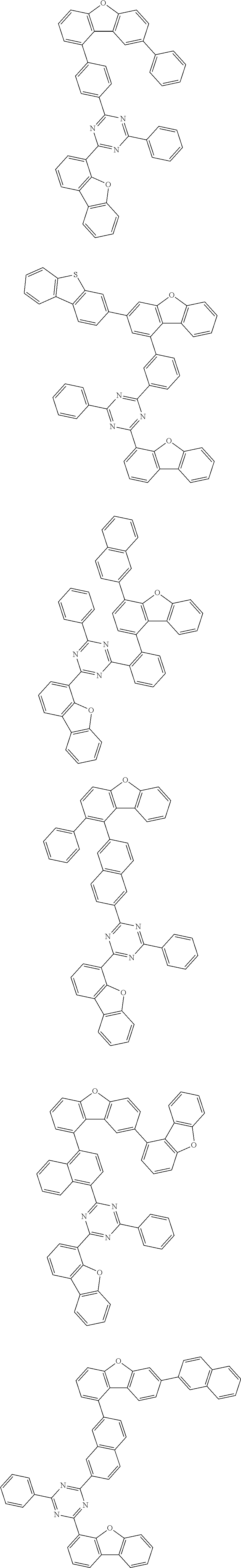

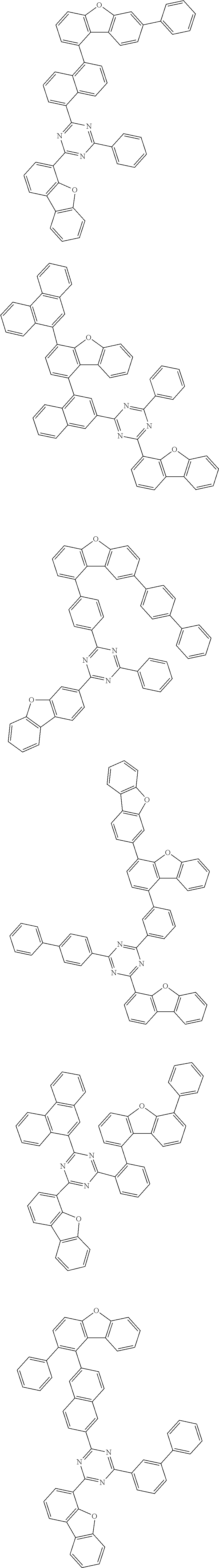

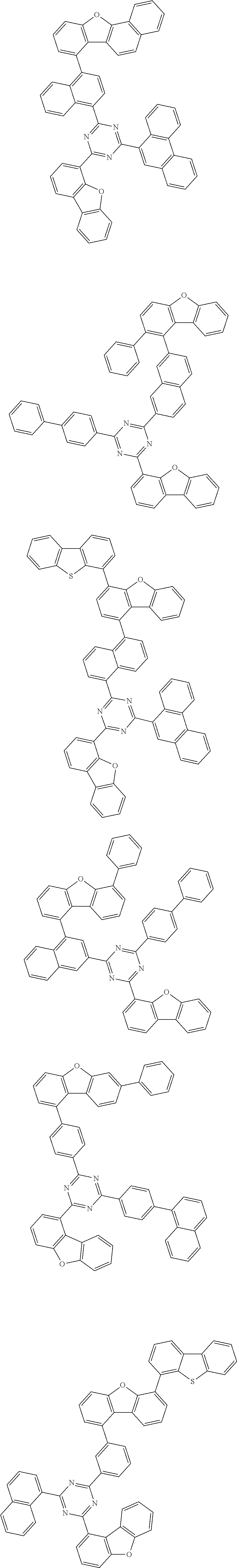

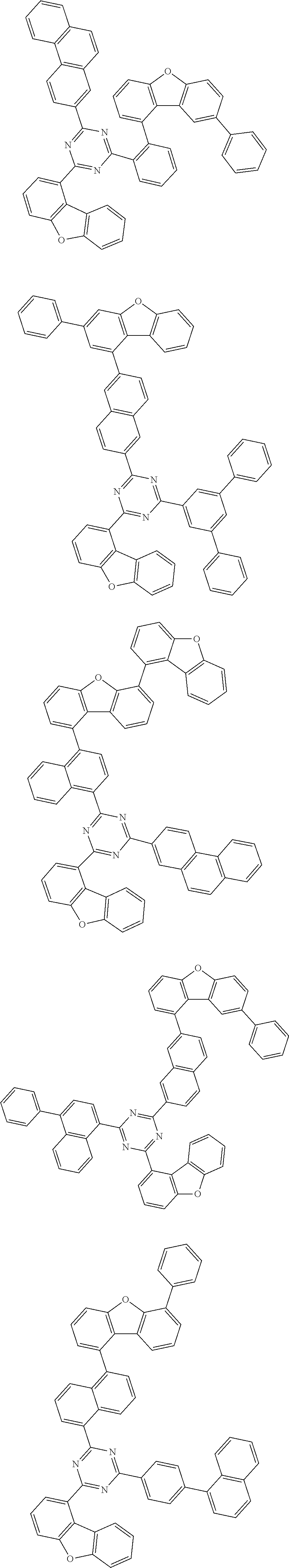

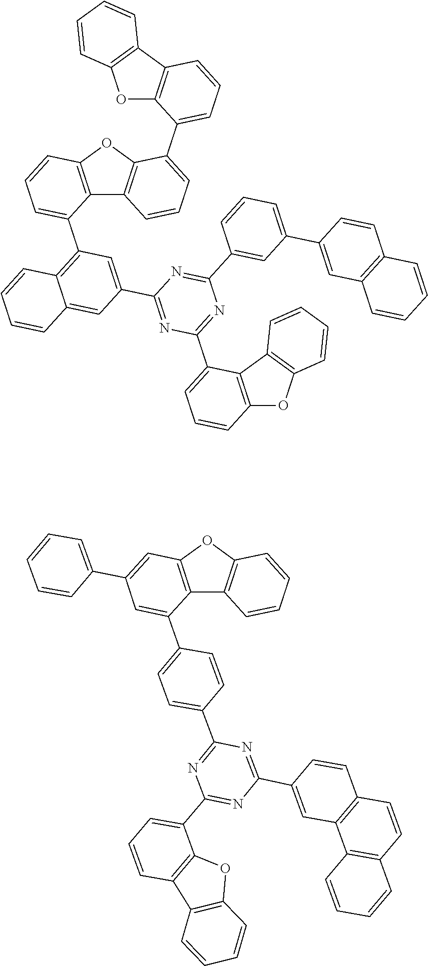 [SMILES: c1cc(-c2ccc3ccccc3c2)cc(-c2nc(-c3cc(-c4cccc5oc6c(-c7cccc8c7oc7ccccc78)cccc6c45)c4ccccc4c3)nc(-c3cccc4oc5ccccc5c34)n2)c1 ; c1ccc(-c2cc(-c3ccc(-c4nc(-c5ccc6ccc7ccccc7c6c5)nc(-c5cccc6c5oc5ccccc56)n4)cc3)c3c(c2)oc2ccccc23)cc1]

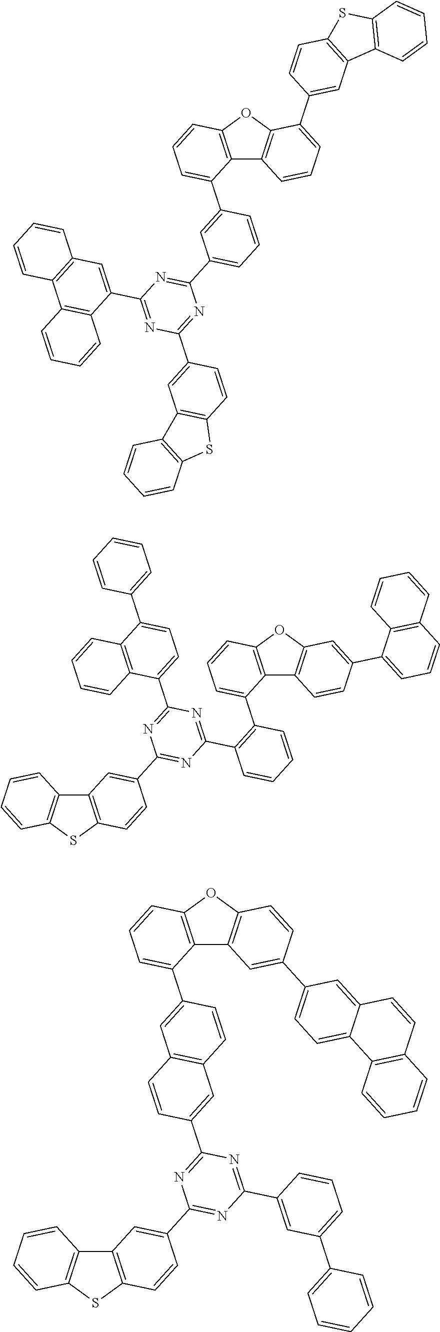 [SMILES: c1cc(-c2nc(-c3ccc4sc5ccccc5c4c3)nc(-c3cc4ccccc4c4ccccc34)n2)cc(-c2cccc3oc4c(-c5ccc6sc7ccccc7c6c5)cccc4c23)c1 ; c1ccc(-c2ccc(-c3nc(-c4ccc5sc6ccccc6c5c4)nc(-c4ccccc4-c4cccc5oc6cc(-c7cccc8ccccc78)ccc6c45)n3)c3ccccc23)cc1 ; c1ccc(-c2cccc(-c3nc(-c4ccc5cc(-c6cccc7oc8ccc(-c9ccc%10c(ccc%11ccccc%11%10)c9)cc8c67)ccc5c4)nc(-c4ccc5sc6ccccc6c5c4)n3)c2)cc1]

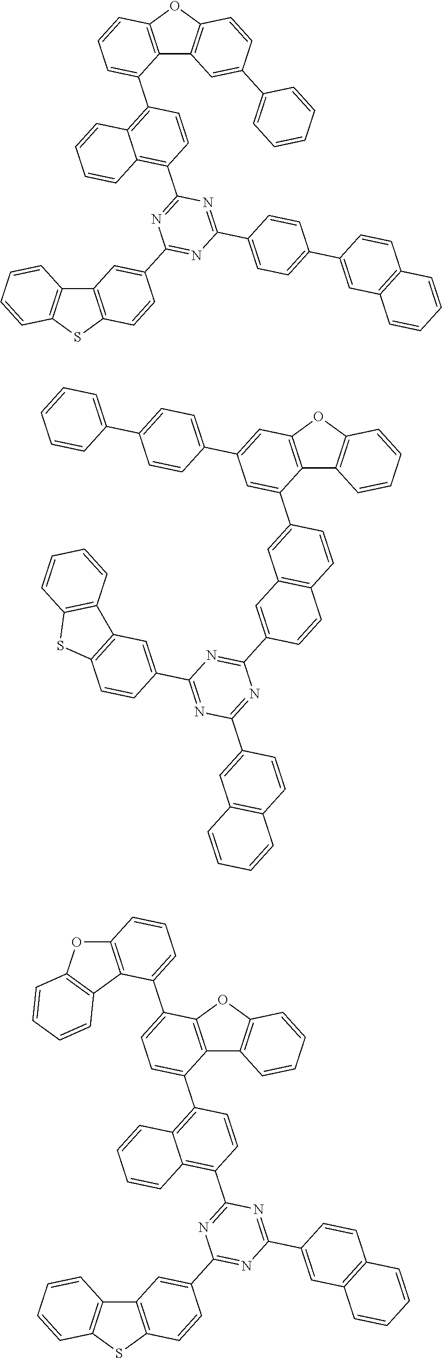 [SMILES: c1ccc(-c2ccc3oc4cccc(-c5ccc(-c6nc(-c7ccc(-c8ccc9ccccc9c8)cc7)nc(-c7ccc8sc9ccccc9c8c7)n6)c6ccccc56)c4c3c2)cc1 ; c1ccc(-c2ccc(-c3cc(-c4ccc5ccc(-c6nc(-c7ccc8ccccc8c7)nc(-c7ccc8sc9ccccc9c8c7)n6)cc5c4)c4c(c3)oc3ccccc34)cc2)cc1 ; c1ccc2cc(-c3nc(-c4ccc5sc6ccccc6c5c4)nc(-c4ccc(-c5ccc(-c6cccc7oc8ccccc8c67)c6oc7ccccc7c56)c5ccccc45)n3)ccc2c1]

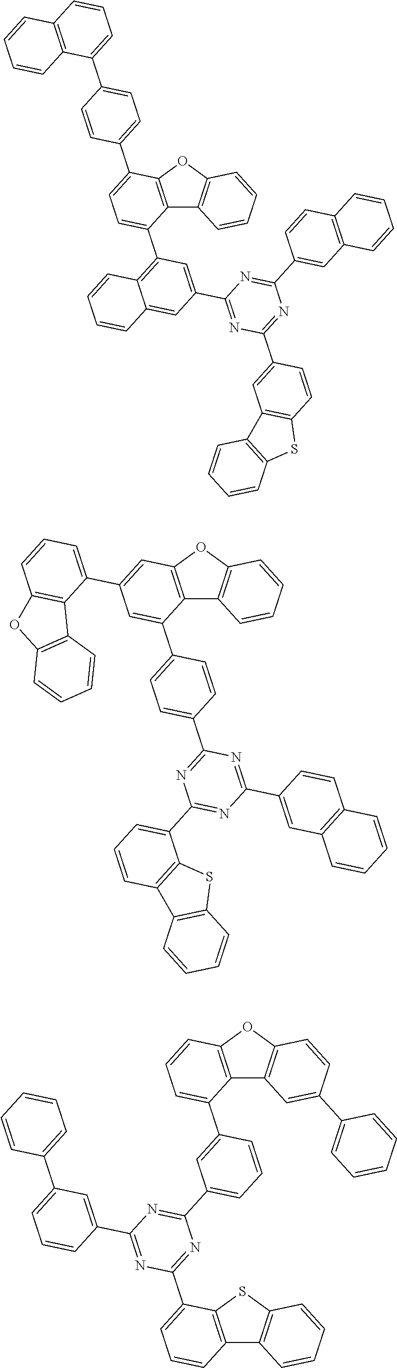 [SMILES: c1ccc2cc(-c3nc(-c4cc(-c5ccc(-c6ccc(-c7cccc8ccccc78)cc6)c6oc7ccccc7c56)c5ccccc5c4)nc(-c4ccc5sc6ccccc6c5c4)n3)ccc2c1 ; c1ccc2cc(-c3nc(-c4ccc(-c5cc(-c6cccc7oc8ccccc8c67)cc6oc7ccccc7c56)cc4)nc(-c4cccc5c4sc4ccccc45)n3)ccc2c1 ; c1ccc(-c2cccc(-c3nc(-c4cccc(-c5cccc6oc7ccc(-c8ccccc8)cc7c56)c4)nc(-c4cccc5c4sc4ccccc45)n3)c2)cc1]

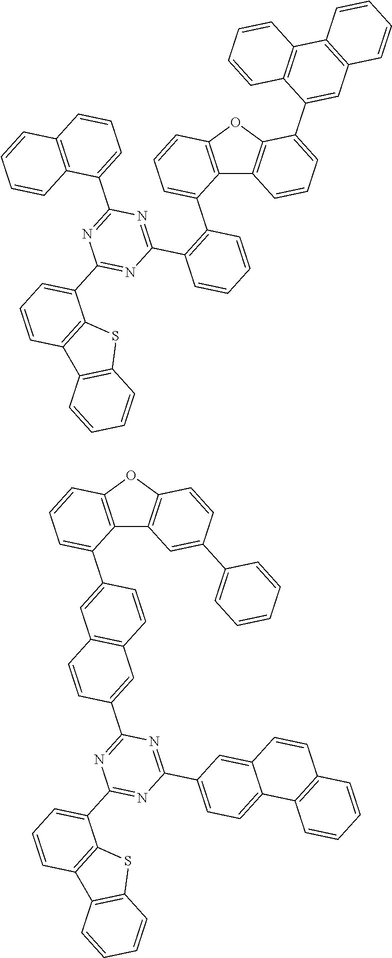 [SMILES: c1ccc(-c2cccc3oc4c(-c5cc6ccccc6c6ccccc56)cccc4c23)c(-c2nc(-c3cccc4ccccc34)nc(-c3cccc4c3sc3ccccc34)n2)c1 ; c1ccc(-c2ccc3oc4cccc(-c5ccc6cc(-c7nc(-c8ccc9c(ccc%10ccccc%109)c8)nc(-c8cccc9c8sc8ccccc89)n7)ccc6c5)c4c3c2)cc1]

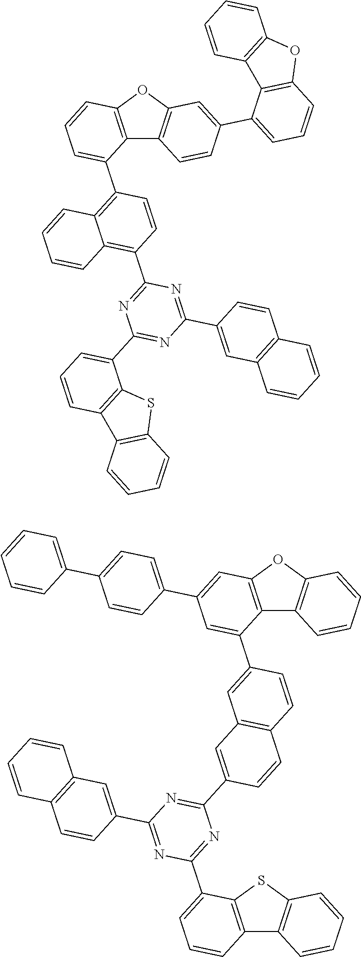 [SMILES: c1ccc2cc(-c3nc(-c4ccc(-c5cccc6oc7cc(-c8cccc9oc%10ccccc%10c89)ccc7c56)c5ccccc45)nc(-c4cccc5c4sc4ccccc45)n3)ccc2c1 ; c1ccc(-c2ccc(-c3cc(-c4ccc5ccc(-c6nc(-c7ccc8ccccc8c7)nc(-c7cccc8c7sc7ccccc78)n6)cc5c4)c4c(c3)oc3ccccc34)cc2)cc1]

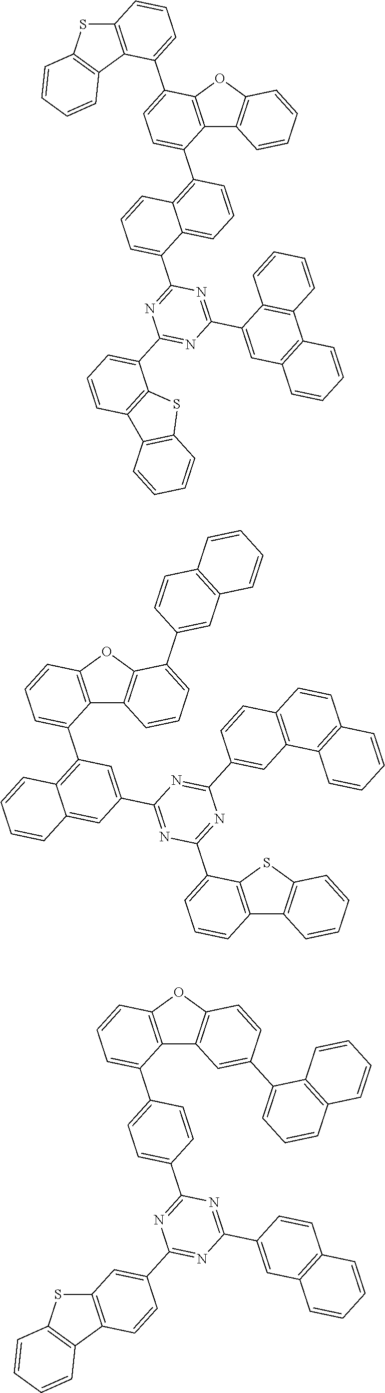 [SMILES: c1ccc2c(c1)cc(-c1nc(-c3cccc4c(-c5ccc(-c6cccc7sc8ccccc8c67)c6oc7ccccc7c56)cccc34)nc(-c3cccc4c3sc3ccccc34)n1)c1ccccc12 ; c1ccc2cc(-c3cccc4c3oc3cccc(-c5cc(-c6nc(-c7ccc8ccc9ccccc9c8c7)nc(-c7cccc8c7sc7ccccc78)n6)cc6ccccc56)c34)ccc2c1 ; c1ccc2cc(-c3nc(-c4ccc(-c5cccc6oc7ccc(-c8cccc9ccccc89)cc7c56)cc4)nc(-c4ccc5c(c4)sc4ccccc45)n3)ccc2c1]

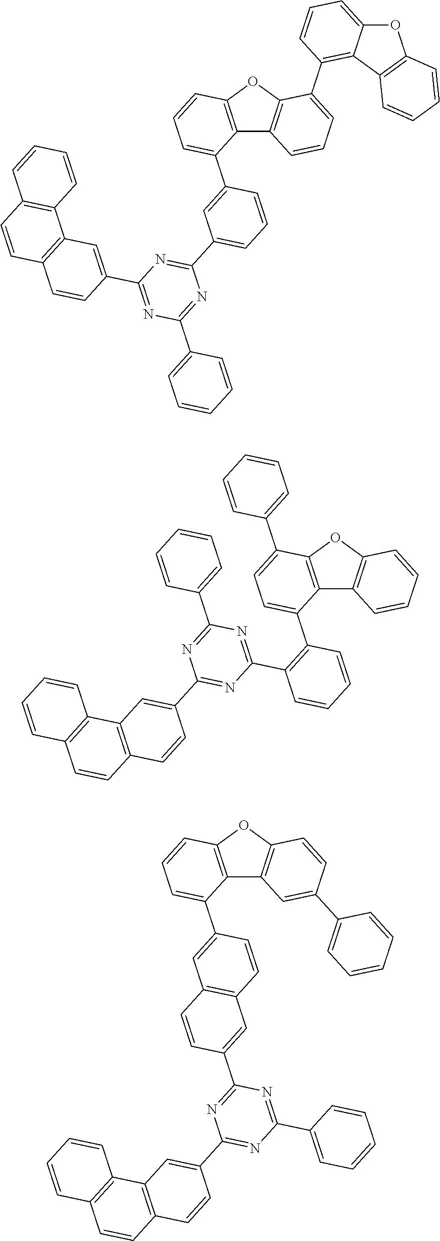 [SMILES: c1ccc(-c2nc(-c3cccc(-c4cccc5oc6c(-c7cccc8oc9ccccc9c78)cccc6c45)c3)nc(-c3ccc4ccc5ccccc5c4c3)n2)cc1 ; c1ccc(-c2nc(-c3ccc4ccc5ccccc5c4c3)nc(-c3ccccc3-c3ccc(-c4ccccc4)c4oc5ccccc5c34)n2)cc1 ; c1ccc(-c2ccc3oc4cccc(-c5ccc6cc(-c7nc(-c8ccccc8)nc(-c8ccc9ccc%10ccccc%10c9c8)n7)ccc6c5)c4c3c2)cc1]

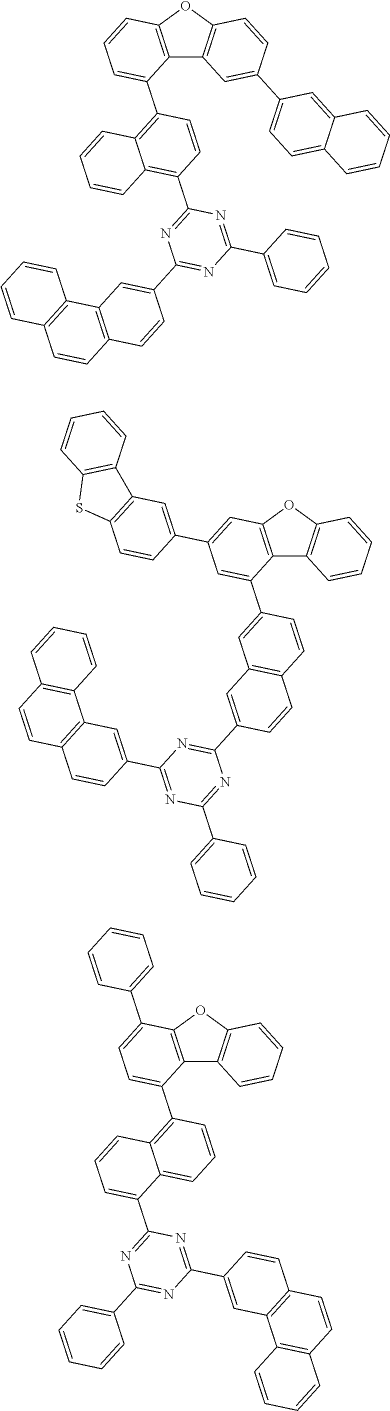 [SMILES: c1ccc(-c2nc(-c3ccc4ccc5ccccc5c4c3)nc(-c3ccc(-c4cccc5oc6ccc(-c7ccc8ccccc8c7)cc6c45)c4ccccc34)n2)cc1 ; c1ccc(-c2nc(-c3ccc4ccc(-c5cc(-c6ccc7sc8ccccc8c7c6)cc6oc7ccccc7c56)cc4c3)nc(-c3ccc4ccc5ccccc5c4c3)n2)cc1 ; c1ccc(-c2nc(-c3ccc4ccc5ccccc5c4c3)nc(-c3cccc4c(-c5ccc(-c6ccccc6)c6oc7ccccc7c56)cccc34)n2)cc1]

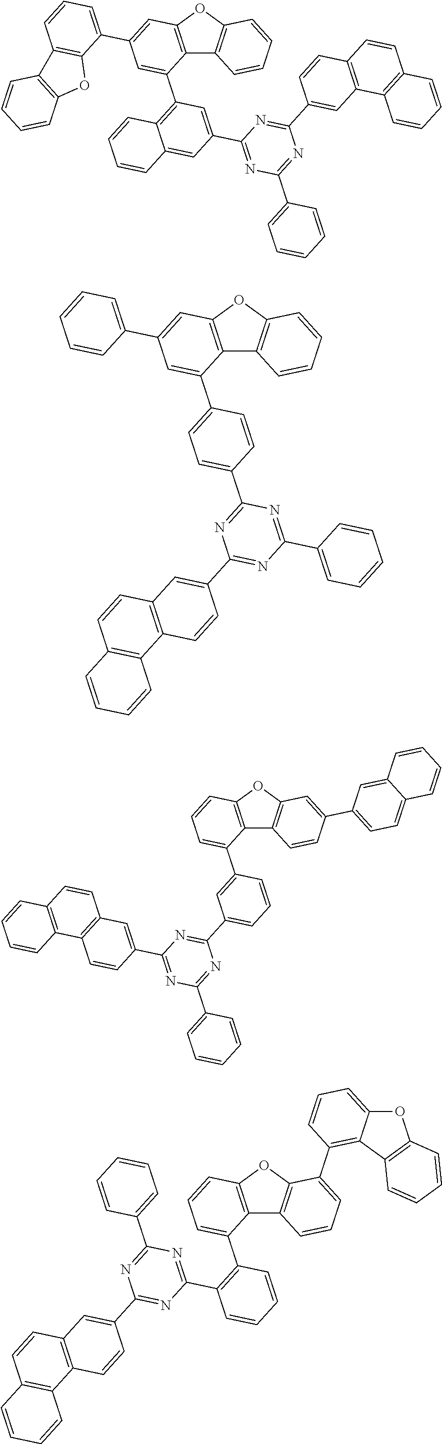 [SMILES: c1ccc(-c2nc(-c3cc(-c4cc(-c5cccc6c5oc5ccccc56)cc5oc6ccccc6c45)c4ccccc4c3)nc(-c3ccc4ccc5ccccc5c4c3)n2)cc1 ; c1ccc(-c2cc(-c3ccc(-c4nc(-c5ccccc5)nc(-c5ccc6c(ccc7ccccc76)c5)n4)cc3)c3c(c2)oc2ccccc23)cc1 ; c1ccc(-c2nc(-c3cccc(-c4cccc5oc6cc(-c7ccc8ccccc8c7)ccc6c45)c3)nc(-c3ccc4c(ccc5ccccc54)c3)n2)cc1 ; c1ccc(-c2nc(-c3ccc4c(ccc5ccccc54)c3)nc(-c3ccccc3-c3cccc4oc5c(-c6cccc7oc8ccccc8c67)cccc5c34)n2)cc1]

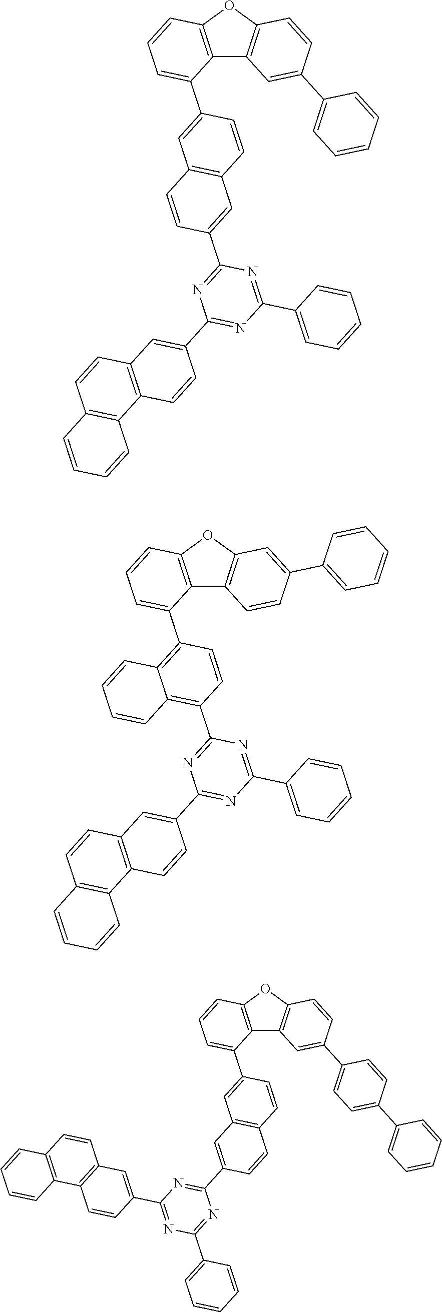 [SMILES: c1ccc(-c2ccc3oc4cccc(-c5ccc6cc(-c7nc(-c8ccccc8)nc(-c8ccc9c(ccc%10ccccc%109)c8)n7)ccc6c5)c4c3c2)cc1 ; c1ccc(-c2ccc3c(c2)oc2cccc(-c4ccc(-c5nc(-c6ccccc6)nc(-c6ccc7c(ccc8ccccc87)c6)n5)c5ccccc45)c23)cc1 ; c1ccc(-c2ccc(-c3ccc4oc5cccc(-c6ccc7ccc(-c8nc(-c9ccccc9)nc(-c9ccc%10c(ccc%11ccccc%11%10)c9)n8)cc7c6)c5c4c3)cc2)cc1]

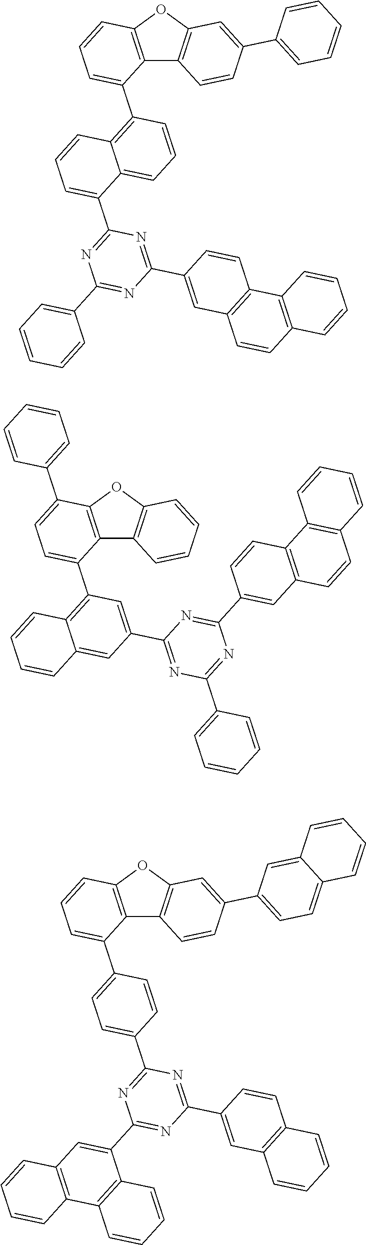

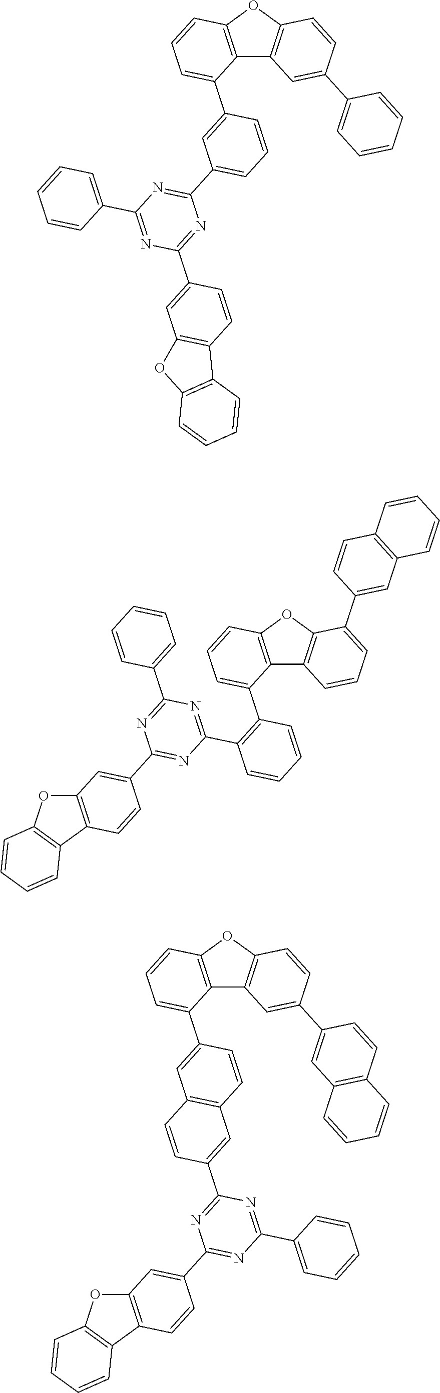 [SMILES: c1ccc(-c2ccc3oc4cccc(-c5cccc(-c6nc(-c7ccccc7)nc(-c7ccc8c(c7)oc7ccccc78)n6)c5)c4c3c2)cc1 ; c1ccc(-c2nc(-c3ccc4c(c3)oc3ccccc34)nc(-c3ccccc3-c3cccc4oc5c(-c6ccc7ccccc7c6)cccc5c34)n2)cc1 ; c1ccc(-c2nc(-c3ccc4cc(-c5cccc6oc7ccc(-c8ccc9ccccc9c8)cc7c56)ccc4c3)nc(-c3ccc4c(c3)oc3ccccc34)n2)cc1]

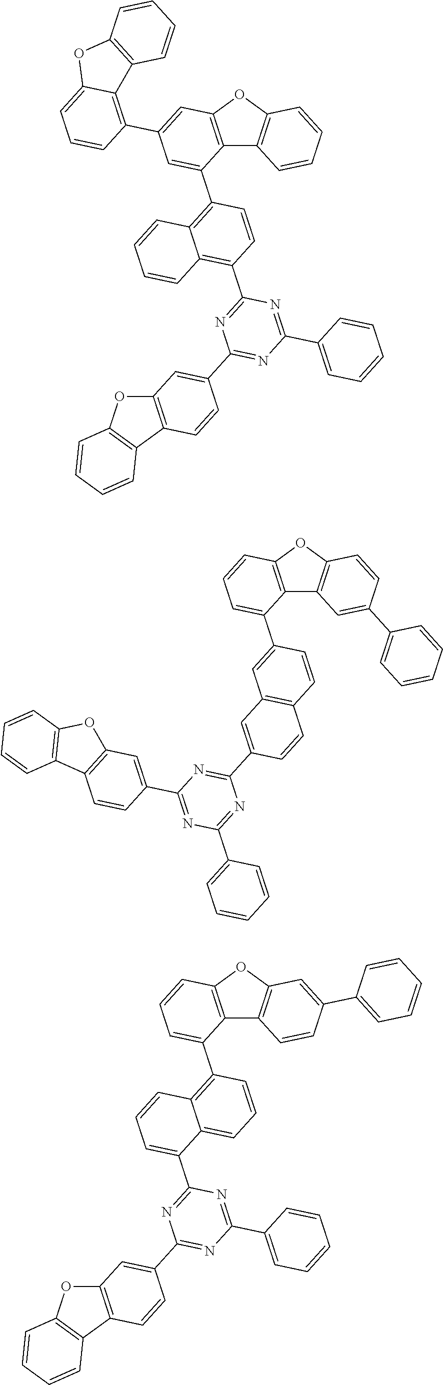 [SMILES: c1ccc(-c2nc(-c3ccc4c(c3)oc3ccccc34)nc(-c3ccc(-c4cc(-c5cccc6oc7ccccc7c56)cc5oc6ccccc6c45)c4ccccc34)n2)cc1 ; c1ccc(-c2ccc3oc4cccc(-c5ccc6ccc(-c7nc(-c8ccccc8)nc(-c8ccc9c(c8)oc8ccccc89)n7)cc6c5)c4c3c2)cc1 ; c1ccc(-c2ccc3c(c2)oc2cccc(-c4cccc5c(-c6nc(-c7ccccc7)nc(-c7ccc8c(c7)oc7ccccc78)n6)cccc45)c23)cc1]

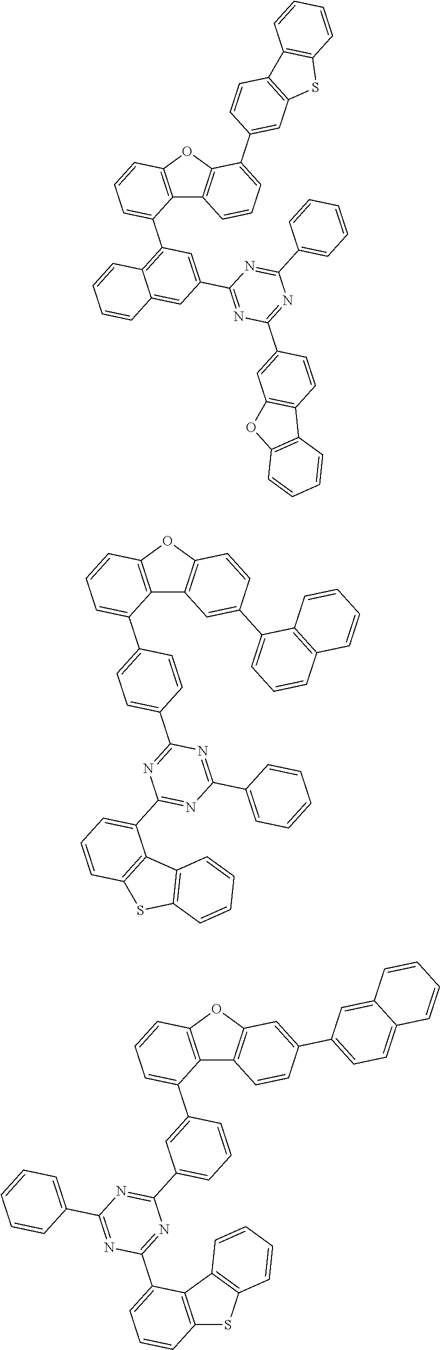 [SMILES: c1ccc(-c2nc(-c3cc(-c4cccc5oc6c(-c7ccc8c(c7)sc7ccccc78)cccc6c45)c4ccccc4c3)nc(-c3ccc4c(c3)oc3ccccc34)n2)cc1 ; c1ccc(-c2nc(-c3ccc(-c4cccc5oc6ccc(-c7cccc8ccccc78)cc6c45)cc3)nc(-c3cccc4sc5ccccc5c34)n2)cc1 ; c1ccc(-c2nc(-c3cccc(-c4cccc5oc6cc(-c7ccc8ccccc8c7)ccc6c45)c3)nc(-c3cccc4sc5ccccc5c34)n2)cc1]

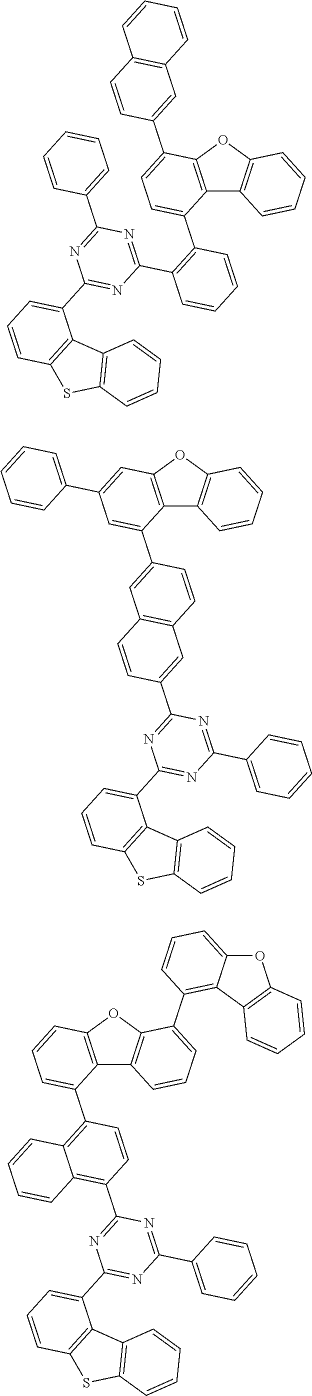

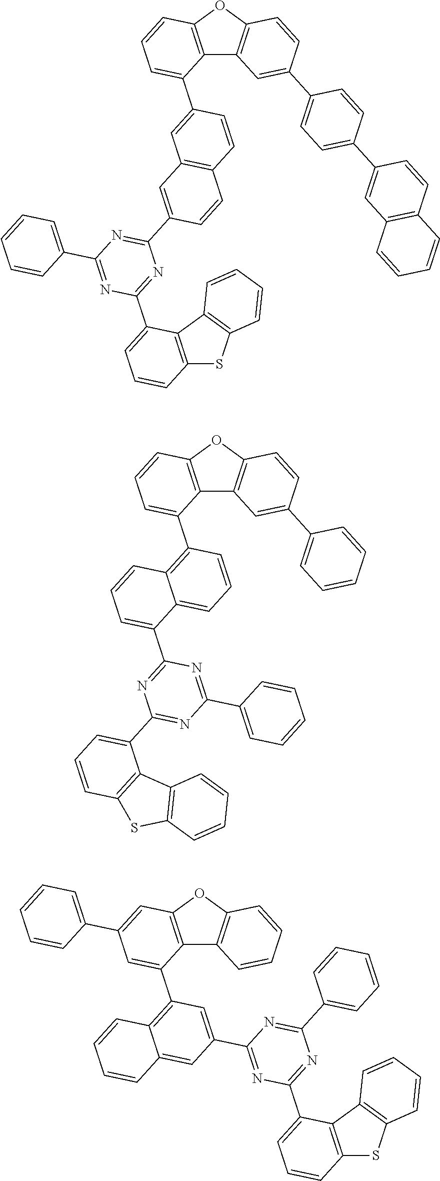 [SMILES: c1ccc(-c2nc(-c3ccc4ccc(-c5cccc6oc7ccc(-c8ccc(-c9ccc%10ccccc%10c9)cc8)cc7c56)cc4c3)nc(-c3cccc4sc5ccccc5c34)n2)cc1 ; c1ccc(-c2ccc3oc4cccc(-c5cccc6c(-c7nc(-c8ccccc8)nc(-c8cccc9sc%10ccccc%10c89)n7)cccc56)c4c3c2)cc1 ; c1ccc(-c2cc(-c3cc(-c4nc(-c5ccccc5)nc(-c5cccc6sc7ccccc7c56)n4)cc4ccccc34)c3c(c2)oc2ccccc23)cc1]

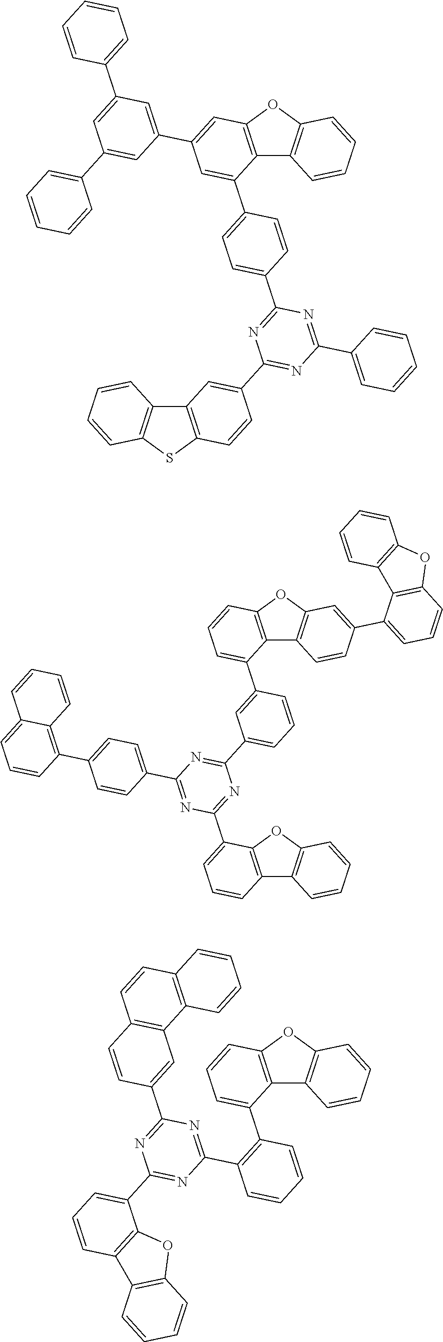 [SMILES: c1ccc(-c2cc(-c3ccccc3)cc(-c3cc(-c4ccc(-c5nc(-c6ccccc6)nc(-c6ccc7sc8ccccc8c7c6)n5)cc4)c4c(c3)oc3ccccc34)c2)cc1 ; c1cc(-c2nc(-c3ccc(-c4cccc5ccccc45)cc3)nc(-c3cccc4c3oc3ccccc34)n2)cc(-c2cccc3oc4cc(-c5cccc6oc7ccccc7c56)ccc4c23)c1 ; c1ccc(-c2cccc3oc4ccccc4c23)c(-c2nc(-c3ccc4ccc5ccccc5c4c3)nc(-c3cccc4c3oc3ccccc34)n2)c1]

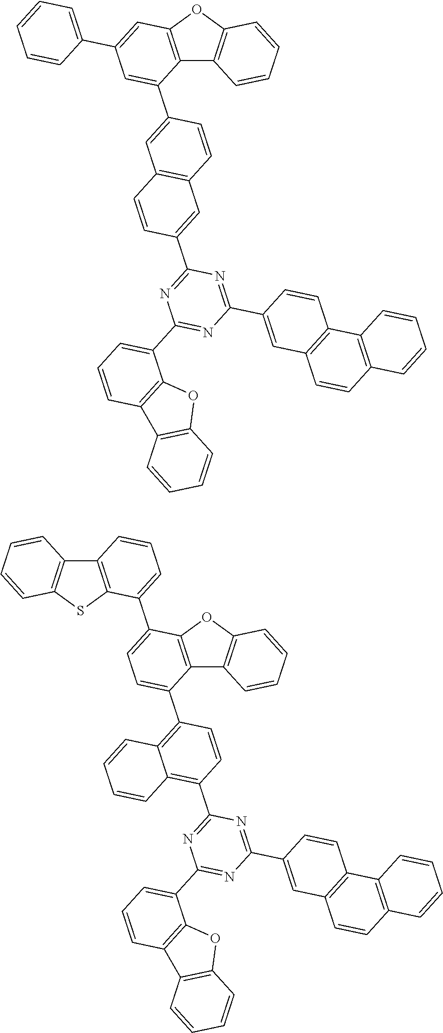 [SMILES: c1ccc(-c2cc(-c3ccc4cc(-c5nc(-c6ccc7c(ccc8ccccc87)c6)nc(-c6cccc7c6oc6ccccc67)n5)ccc4c3)c3c(c2)oc2ccccc23)cc1 ; c1ccc2c(c1)ccc1cc(-c3nc(-c4ccc(-c5ccc(-c6cccc7c6sc6ccccc67)c6oc7ccccc7c56)c5ccccc45)nc(-c4cccc5c4oc4ccccc45)n3)ccc12]

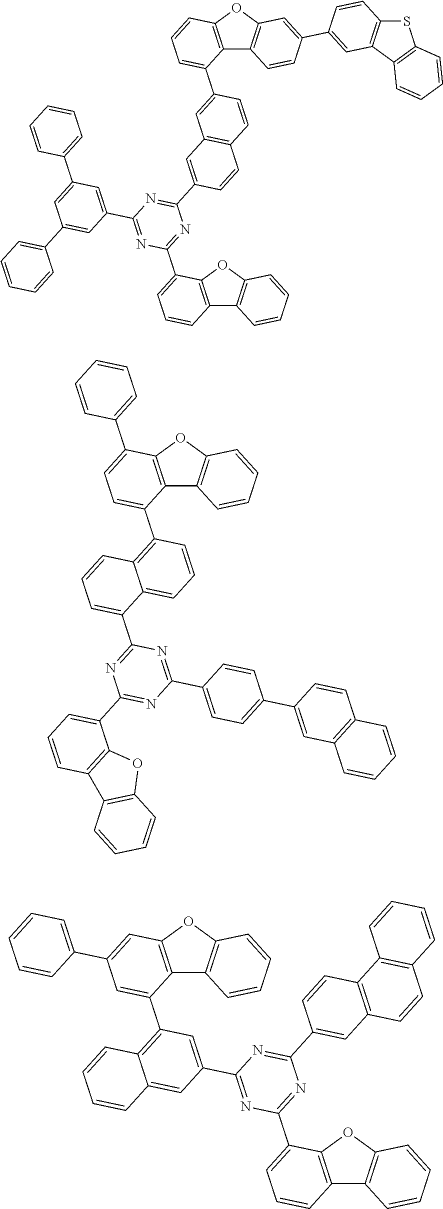 [SMILES: c1ccc(-c2cc(-c3ccccc3)cc(-c3nc(-c4ccc5ccc(-c6cccc7oc8cc(-c9ccc%10sc%11ccccc%11c%10c9)ccc8c67)cc5c4)nc(-c4cccc5c4oc4ccccc45)n3)c2)cc1 ; c1ccc(-c2ccc(-c3cccc4c(-c5nc(-c6ccc(-c7ccc8ccccc8c7)cc6)nc(-c6cccc7c6oc6ccccc67)n5)cccc34)c3c2oc2ccccc23)cc1 ; c1ccc(-c2cc(-c3cc(-c4nc(-c5ccc6c(ccc7ccccc76)c5)nc(-c5cccc6c5oc5ccccc56)n4)cc4ccccc34)c3c(c2)oc2ccccc23)cc1]

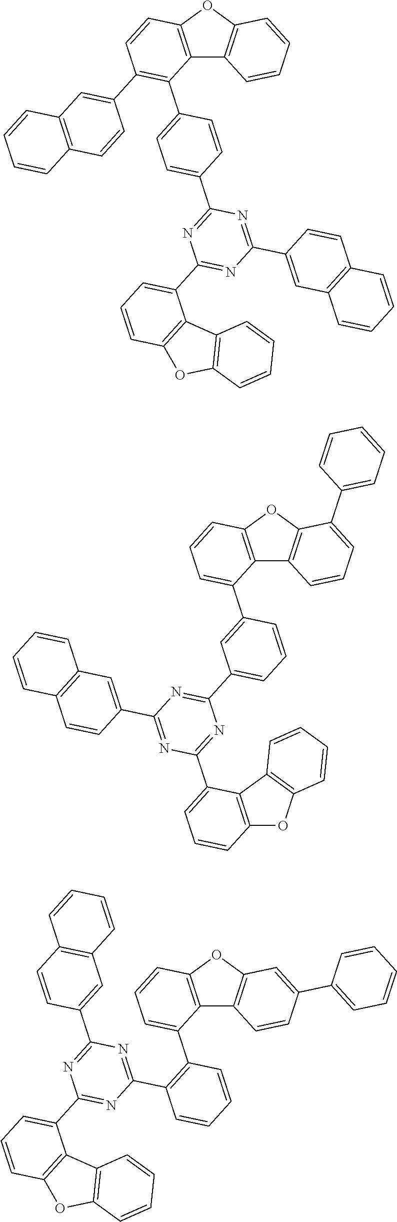 [SMILES: c1ccc2cc(-c3nc(-c4ccc(-c5c(-c6ccc7ccccc7c6)ccc6oc7ccccc7c56)cc4)nc(-c4cccc5oc6ccccc6c45)n3)ccc2c1 ; c1ccc(-c2cccc3c2oc2cccc(-c4cccc(-c5nc(-c6ccc7ccccc7c6)nc(-c6cccc7oc8ccccc8c67)n5)c4)c23)cc1 ; c1ccc(-c2ccc3c(c2)oc2cccc(-c4ccccc4-c4nc(-c5ccc6ccccc6c5)nc(-c5cccc6oc7ccccc7c56)n4)c23)cc1]

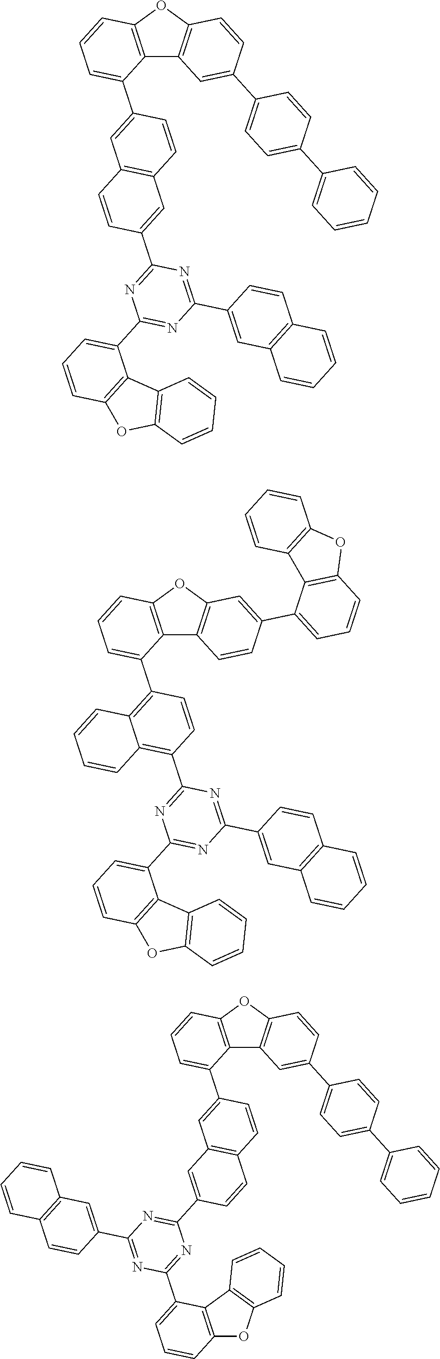 [SMILES: c1ccc(-c2ccc(-c3ccc4oc5cccc(-c6ccc7cc(-c8nc(-c9ccc%10ccccc%10c9)nc(-c9cccc%10oc%11ccccc%11c9%10)n8)ccc7c6)c5c4c3)cc2)cc1 ; c1ccc2cc(-c3nc(-c4ccc(-c5cccc6oc7cc(-c8cccc9oc%10ccccc%10c89)ccc7c56)c5ccccc45)nc(-c4cccc5oc6ccccc6c45)n3)ccc2c1 ; c1ccc(-c2ccc(-c3ccc4oc5cccc(-c6ccc7ccc(-c8nc(-c9ccc%10ccccc%10c9)nc(-c9cccc%10oc%11ccccc%11c9%10)n8)cc7c6)c5c4c3)cc2)cc1]

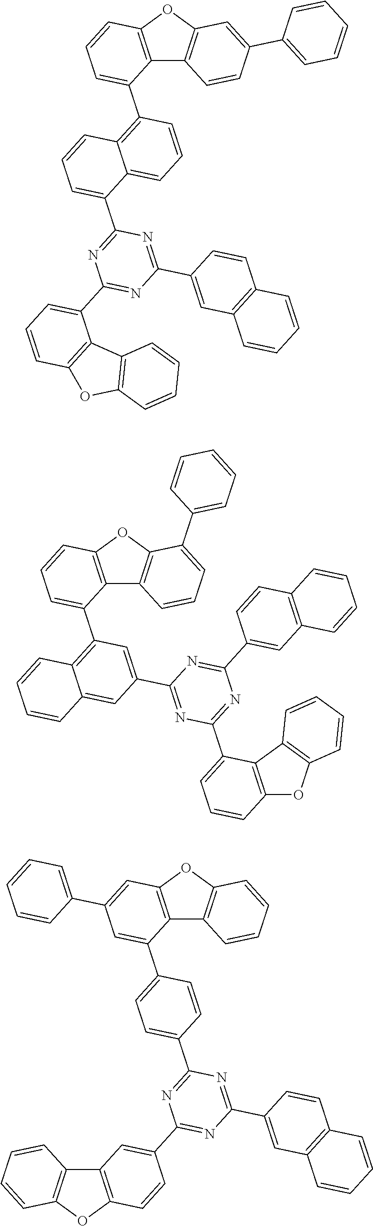 [SMILES: c1ccc(-c2ccc3c(c2)oc2cccc(-c4cccc5c(-c6nc(-c7ccc8ccccc8c7)nc(-c7cccc8oc9ccccc9c78)n6)cccc45)c23)cc1 ; c1ccc(-c2cccc3c2oc2cccc(-c4cc(-c5nc(-c6ccc7ccccc7c6)nc(-c6cccc7oc8ccccc8c67)n5)cc5ccccc45)c23)cc1 ; c1ccc(-c2cc(-c3ccc(-c4nc(-c5ccc6ccccc6c5)nc(-c5ccc6oc7ccccc7c6c5)n4)cc3)c3c(c2)oc2ccccc23)cc1]

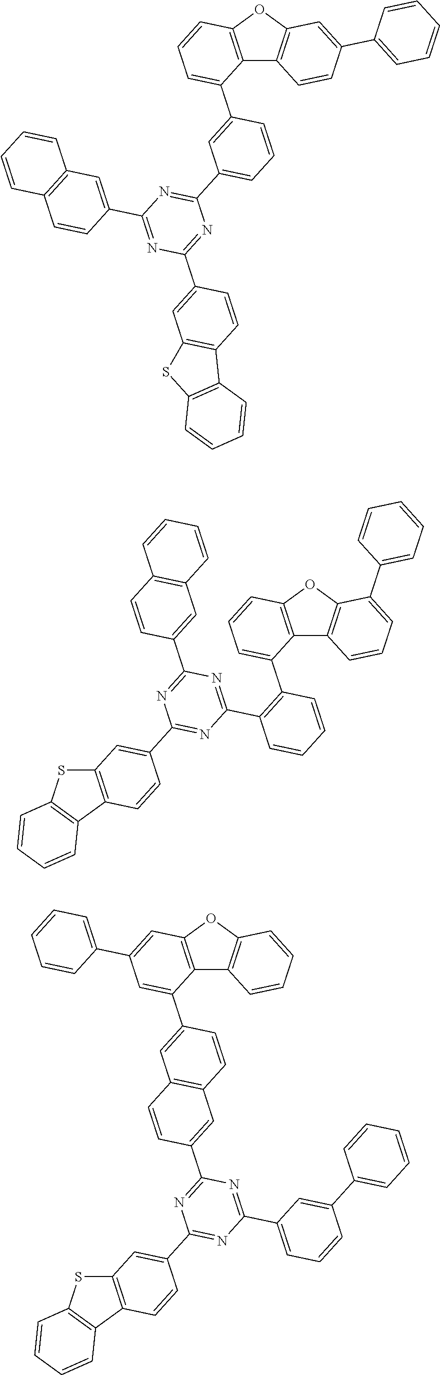

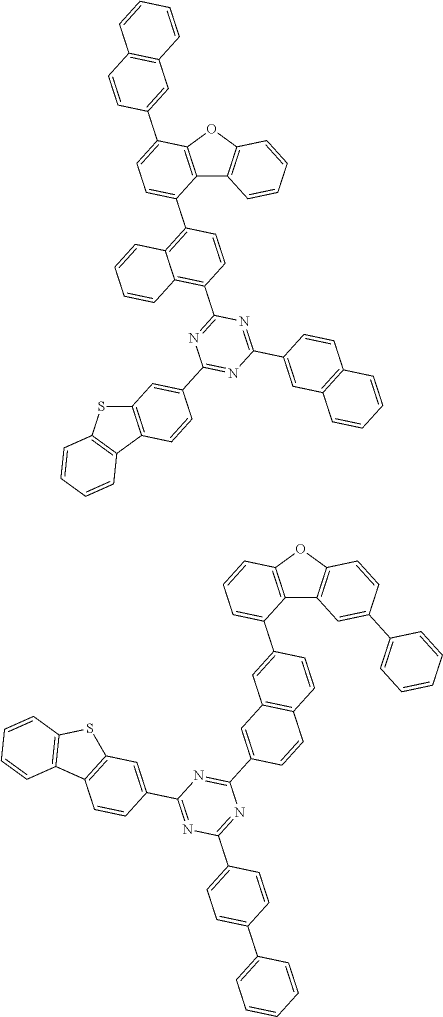 [SMILES: c1ccc2cc(-c3nc(-c4ccc5c(c4)sc4ccccc45)nc(-c4ccc(-c5ccc(-c6ccc7ccccc7c6)c6oc7ccccc7c56)c5ccccc45)n3)ccc2c1 ; c1ccc(-c2ccc(-c3nc(-c4ccc5ccc(-c6cccc7oc8ccc(-c9ccccc9)cc8c67)cc5c4)nc(-c4ccc5c(c4)sc4ccccc45)n3)cc2)cc1]

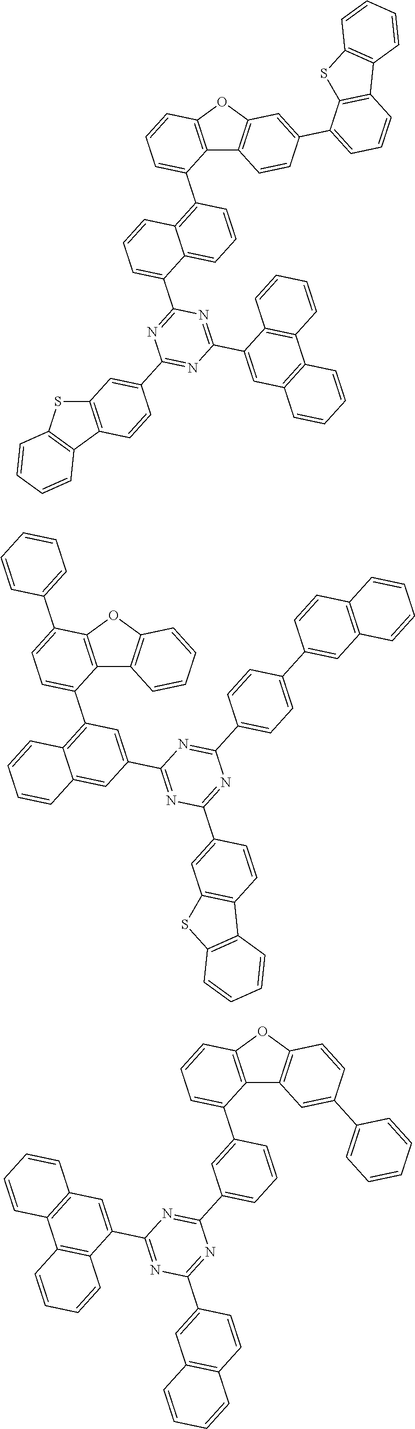

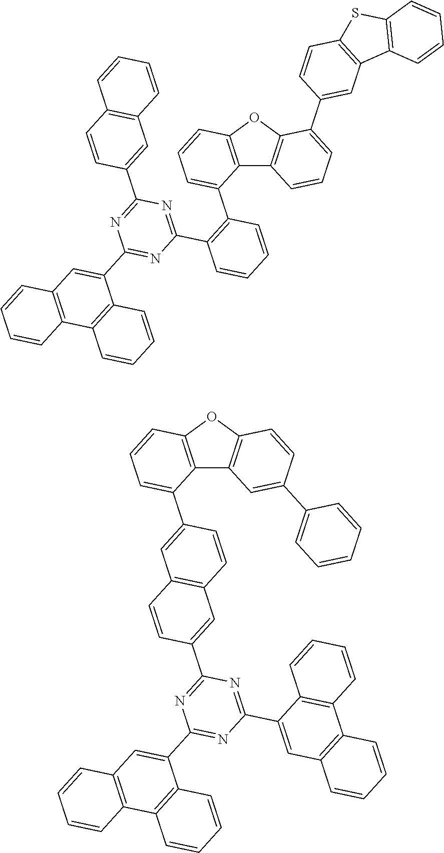 [SMILES: c1ccc(-c2cccc3oc4c(-c5ccc6sc7ccccc7c6c5)cccc4c23)c(-c2nc(-c3ccc4ccccc4c3)nc(-c3cc4ccccc4c4ccccc34)n2)c1 ; c1ccc(-c2ccc3oc4cccc(-c5ccc6cc(-c7nc(-c8cc9ccccc9c9ccccc89)nc(-c8cc9ccccc9c9ccccc89)n7)ccc6c5)c4c3c2)cc1]

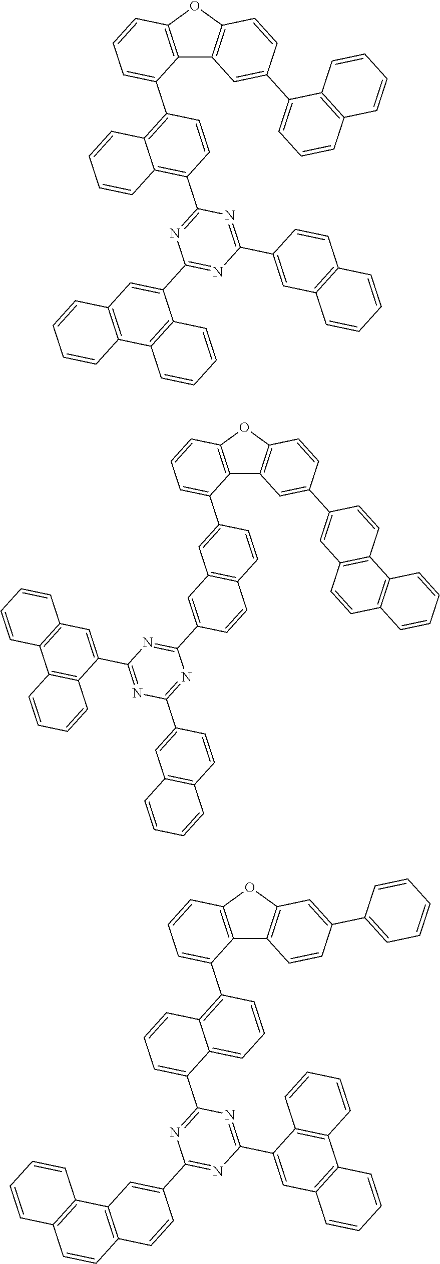 [SMILES: c1ccc2cc(-c3nc(-c4ccc(-c5cccc6oc7ccc(-c8cccc9ccccc89)cc7c56)c5ccccc45)nc(-c4cc5ccccc5c5ccccc45)n3)ccc2c1 ; c1ccc2cc(-c3nc(-c4ccc5ccc(-c6cccc7oc8ccc(-c9ccc%10c(ccc%11ccccc%11%10)c9)cc8c67)cc5c4)nc(-c4cc5ccccc5c5ccccc45)n3)ccc2c1 ; c1ccc(-c2ccc3c(c2)oc2cccc(-c4cccc5c(-c6nc(-c7ccc8ccc9ccccc9c8c7)nc(-c7cc8ccccc8c8ccccc78)n6)cccc45)c23)cc1]

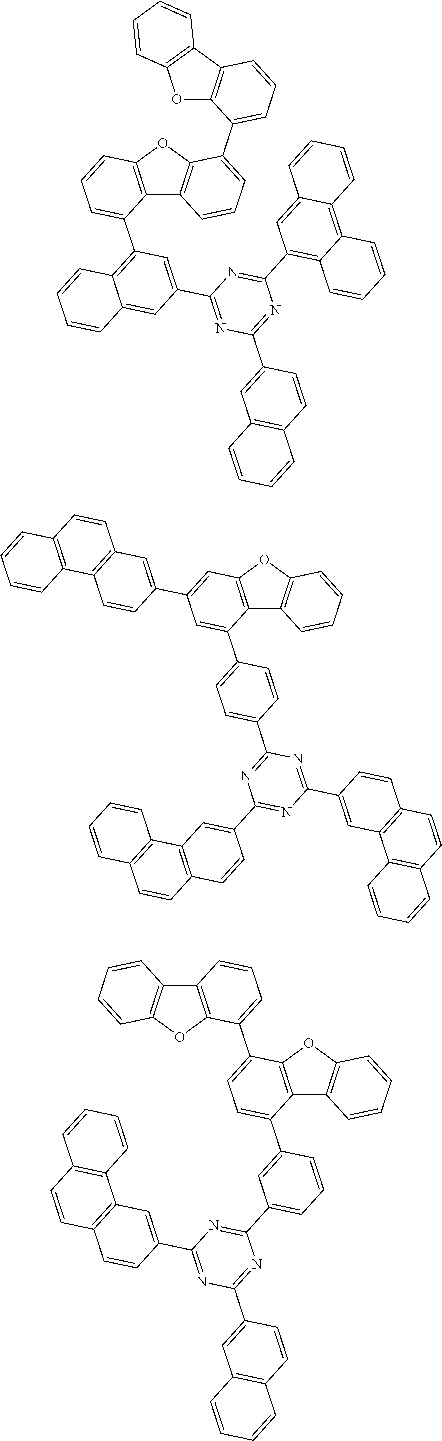 [SMILES: c1ccc2cc(-c3nc(-c4cc(-c5cccc6oc7c(-c8cccc9c8oc8ccccc89)cccc7c56)c5ccccc5c4)nc(-c4cc5ccccc5c5ccccc45)n3)ccc2c1 ; c1ccc2c(c1)ccc1cc(-c3cc(-c4ccc(-c5nc(-c6ccc7ccc8ccccc8c7c6)nc(-c6ccc7ccc8ccccc8c7c6)n5)cc4)c4c(c3)oc3ccccc34)ccc12 ; c1cc(-c2nc(-c3ccc4ccccc4c3)nc(-c3ccc4ccc5ccccc5c4c3)n2)cc(-c2ccc(-c3cccc4c3oc3ccccc34)c3oc4ccccc4c23)c1]

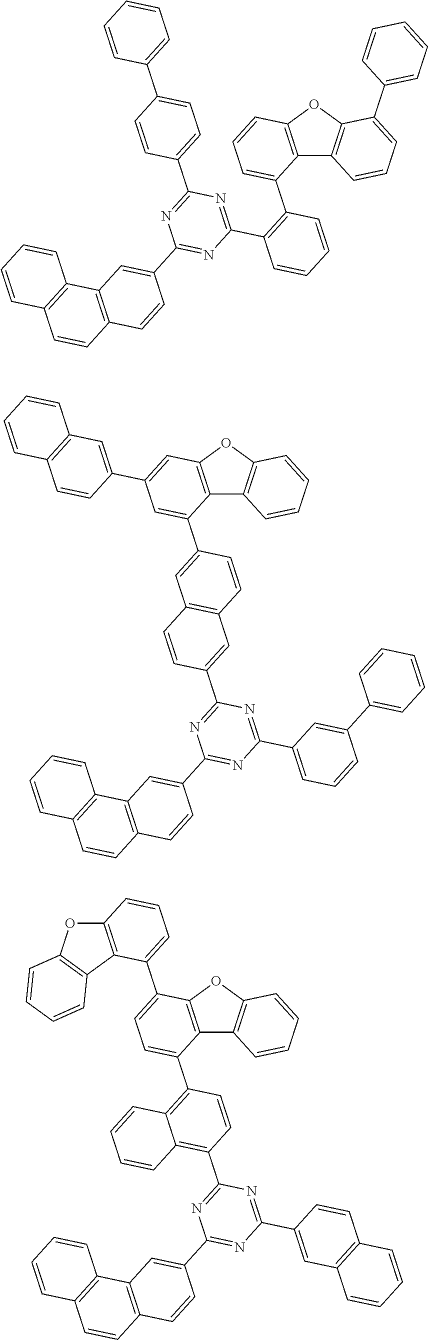

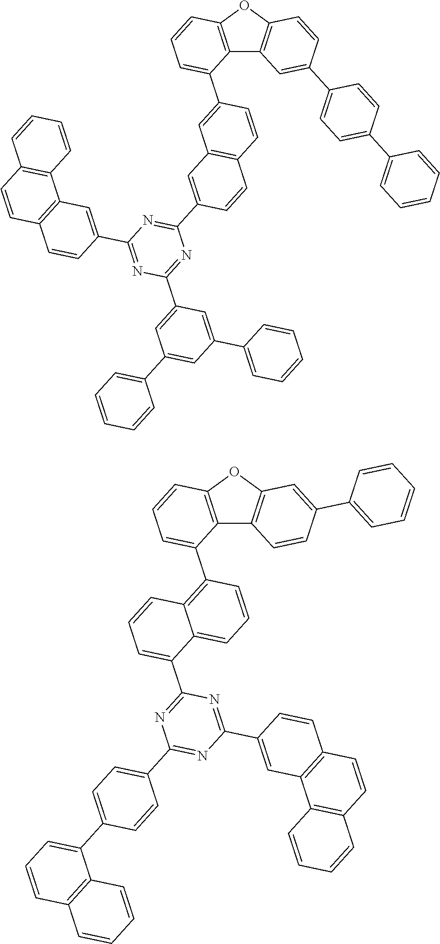 [SMILES: c1ccc(-c2ccc(-c3ccc4oc5cccc(-c6ccc7ccc(-c8nc(-c9cc(-c%10ccccc%10)cc(-c%10ccccc%10)c9)nc(-c9ccc%10ccc%11ccccc%11c%10c9)n8)cc7c6)c5c4c3)cc2)cc1 ; c1ccc(-c2ccc3c(c2)oc2cccc(-c4cccc5c(-c6nc(-c7ccc(-c8cccc9ccccc89)cc7)nc(-c7ccc8ccc9ccccc9c8c7)n6)cccc45)c23)cc1]

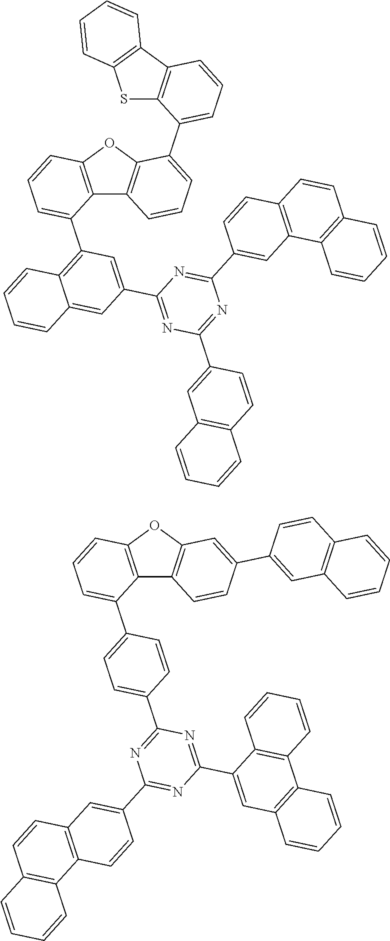 [SMILES: c1ccc2cc(-c3nc(-c4cc(-c5cccc6oc7c(-c8cccc9c8sc8ccccc89)cccc7c56)c5ccccc5c4)nc(-c4ccc5ccc6ccccc6c5c4)n3)ccc2c1 ; c1ccc2cc(-c3ccc4c(c3)oc3cccc(-c5ccc(-c6nc(-c7ccc8c(ccc9ccccc98)c7)nc(-c7cc8ccccc8c8ccccc78)n6)cc5)c34)ccc2c1]

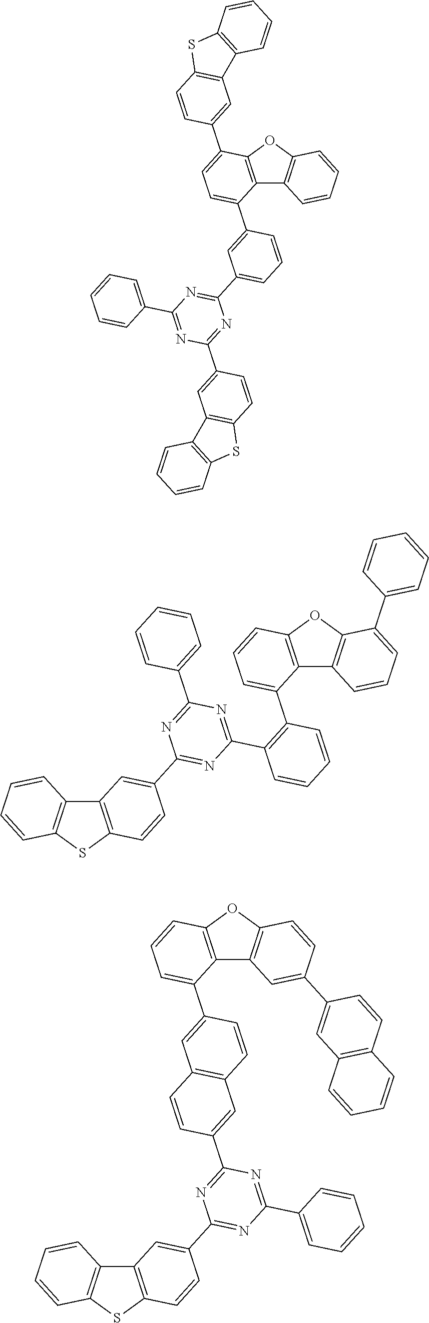 [SMILES: c1ccc(-c2nc(-c3cccc(-c4ccc(-c5ccc6sc7ccccc7c6c5)c5oc6ccccc6c45)c3)nc(-c3ccc4sc5ccccc5c4c3)n2)cc1 ; c1ccc(-c2nc(-c3ccc4sc5ccccc5c4c3)nc(-c3ccccc3-c3cccc4oc5c(-c6ccccc6)cccc5c34)n2)cc1 ; c1ccc(-c2nc(-c3ccc4cc(-c5cccc6oc7ccc(-c8ccc9ccccc9c8)cc7c56)ccc4c3)nc(-c3ccc4sc5ccccc5c4c3)n2)cc1]

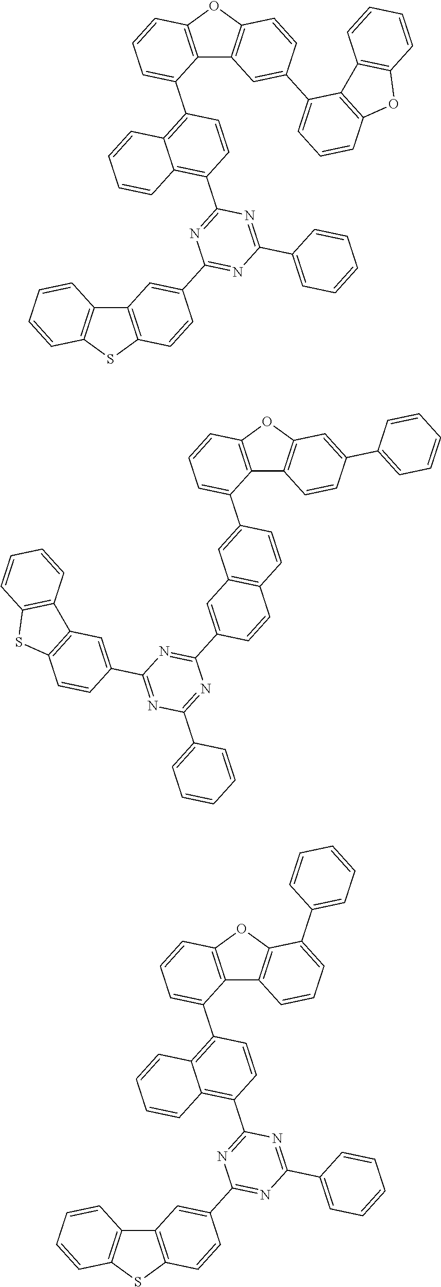 [SMILES: c1ccc(-c2nc(-c3ccc4sc5ccccc5c4c3)nc(-c3ccc(-c4cccc5oc6ccc(-c7cccc8oc9ccccc9c78)cc6c45)c4ccccc34)n2)cc1 ; c1ccc(-c2ccc3c(c2)oc2cccc(-c4ccc5ccc(-c6nc(-c7ccccc7)nc(-c7ccc8sc9ccccc9c8c7)n6)cc5c4)c23)cc1 ; c1ccc(-c2nc(-c3ccc4sc5ccccc5c4c3)nc(-c3ccc(-c4cccc5oc6c(-c7ccccc7)cccc6c45)c4ccccc34)n2)cc1]

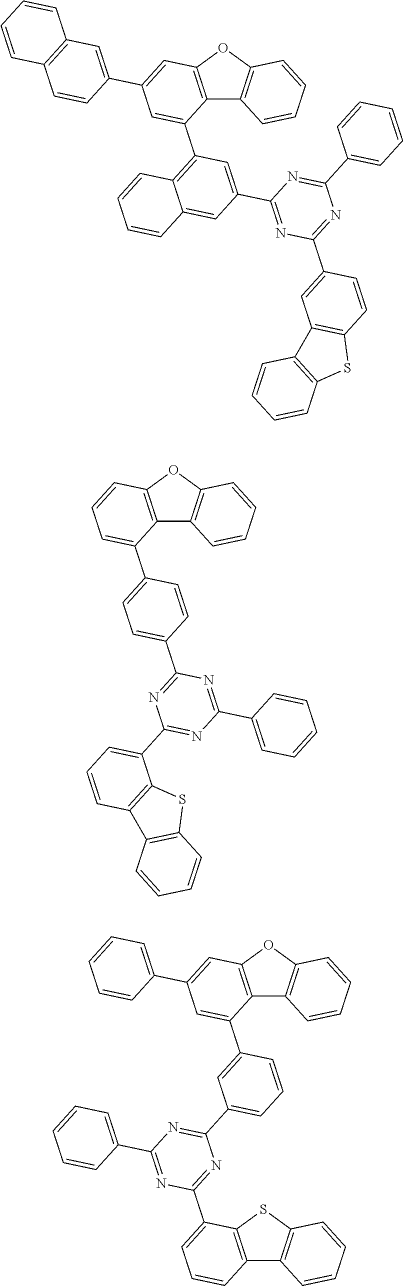 [SMILES: c1ccc(-c2nc(-c3cc(-c4cc(-c5ccc6ccccc6c5)cc5oc6ccccc6c45)c4ccccc4c3)nc(-c3ccc4sc5ccccc5c4c3)n2)cc1 ; c1ccc(-c2nc(-c3ccc(-c4cccc5oc6ccccc6c45)cc3)nc(-c3cccc4c3sc3ccccc34)n2)cc1 ; c1ccc(-c2cc(-c3cccc(-c4nc(-c5ccccc5)nc(-c5cccc6c5sc5ccccc56)n4)c3)c3c(c2)oc2ccccc23)cc1]

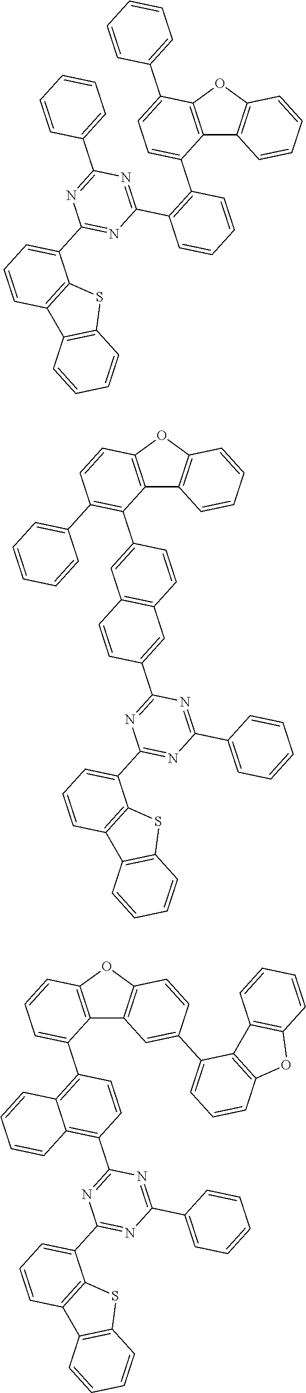 [SMILES: c1ccc(-c2nc(-c3ccccc3-c3ccc(-c4ccccc4)c4oc5ccccc5c34)nc(-c3cccc4c3sc3ccccc34)n2)cc1 ; c1ccc(-c2nc(-c3ccc4cc(-c5c(-c6ccccc6)ccc6oc7ccccc7c56)ccc4c3)nc(-c3cccc4c3sc3ccccc34)n2)cc1 ; c1ccc(-c2nc(-c3ccc(-c4cccc5oc6ccc(-c7cccc8oc9ccccc9c78)cc6c45)c4ccccc34)nc(-c3cccc4c3sc3ccccc34)n2)cc1]

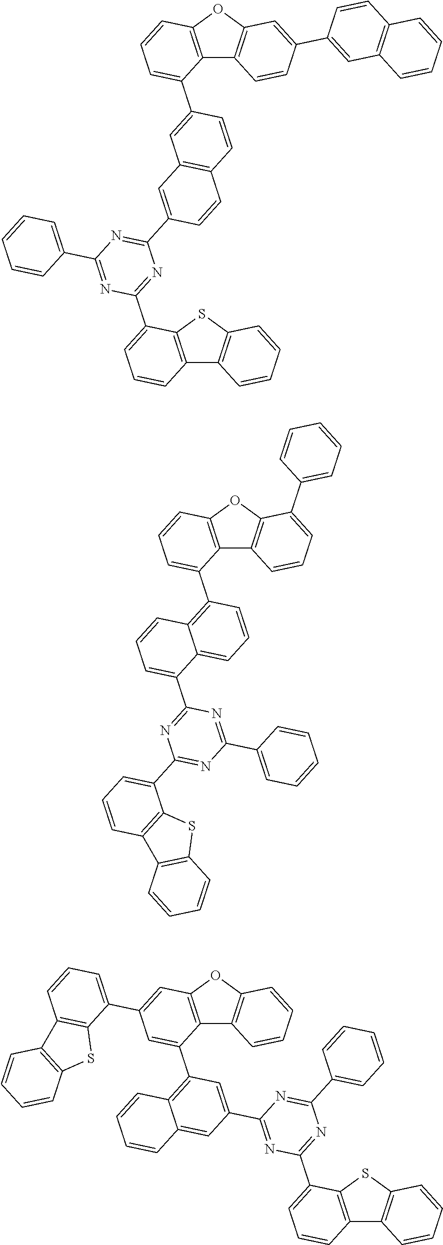 [SMILES: c1ccc(-c2nc(-c3ccc4ccc(-c5cccc6oc7cc(-c8ccc9ccccc9c8)ccc7c56)cc4c3)nc(-c3cccc4c3sc3ccccc34)n2)cc1 ; c1ccc(-c2nc(-c3cccc4c(-c5cccc6oc7c(-c8ccccc8)cccc7c56)cccc34)nc(-c3cccc4c3sc3ccccc34)n2)cc1 ; c1ccc(-c2nc(-c3cc(-c4cc(-c5cccc6c5sc5ccccc56)cc5oc6ccccc6c45)c4ccccc4c3)nc(-c3cccc4c3sc3ccccc34)n2)cc1]

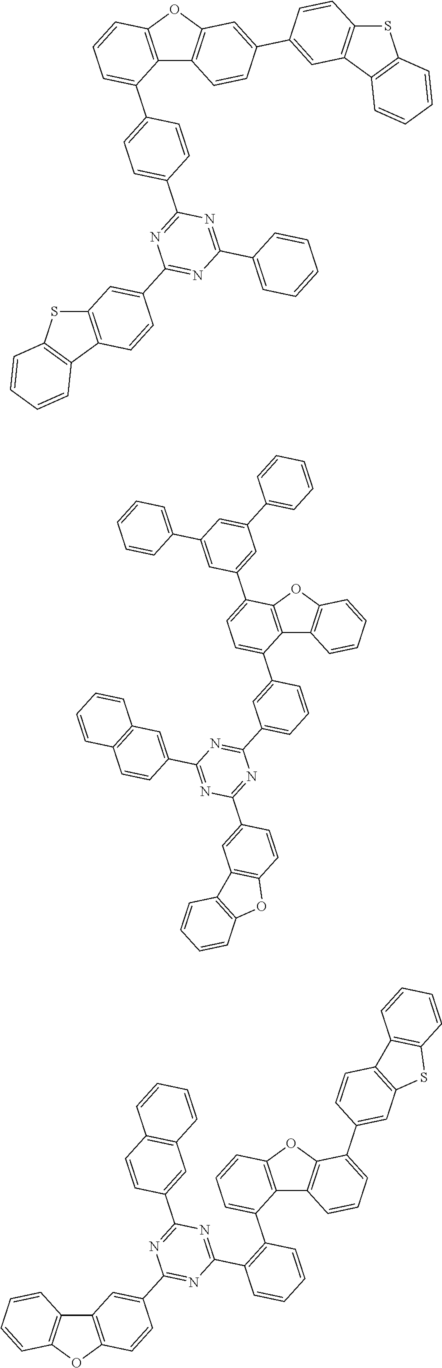 [SMILES: c1ccc(-c2nc(-c3ccc(-c4cccc5oc6cc(-c7ccc8sc9ccccc9c8c7)ccc6c45)cc3)nc(-c3ccc4c(c3)sc3ccccc34)n2)cc1 ; c1ccc(-c2cc(-c3ccccc3)cc(-c3ccc(-c4cccc(-c5nc(-c6ccc7ccccc7c6)nc(-c6ccc7oc8ccccc8c7c6)n5)c4)c4c3oc3ccccc34)c2)cc1 ; c1ccc(-c2cccc3oc4c(-c5ccc6c(c5)sc5ccccc56)cccc4c23)c(-c2nc(-c3ccc4ccccc4c3)nc(-c3ccc4oc5ccccc5c4c3)n2)c1]

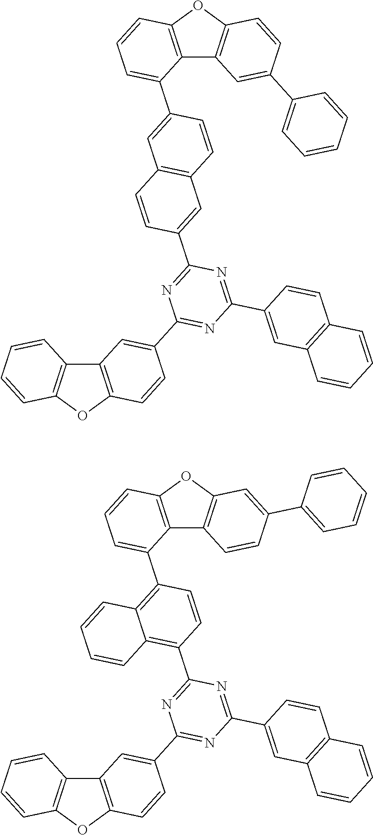 [SMILES: c1ccc(-c2ccc3oc4cccc(-c5ccc6cc(-c7nc(-c8ccc9ccccc9c8)nc(-c8ccc9oc%10ccccc%10c9c8)n7)ccc6c5)c4c3c2)cc1 ; c1ccc(-c2ccc3c(c2)oc2cccc(-c4ccc(-c5nc(-c6ccc7ccccc7c6)nc(-c6ccc7oc8ccccc8c7c6)n5)c5ccccc45)c23)cc1]

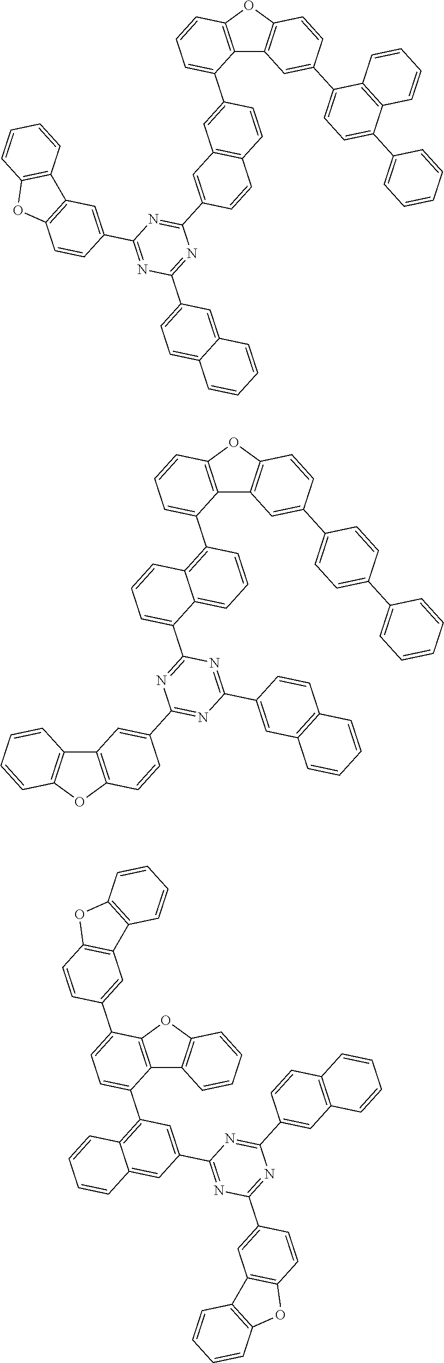 [SMILES: c1ccc(-c2ccc(-c3ccc4oc5cccc(-c6ccc7ccc(-c8nc(-c9ccc%10ccccc%10c9)nc(-c9ccc%10oc%11ccccc%11c%10c9)n8)cc7c6)c5c4c3)c3ccccc23)cc1 ; c1ccc(-c2ccc(-c3ccc4oc5cccc(-c6cccc7c(-c8nc(-c9ccc%10ccccc%10c9)nc(-c9ccc%10oc%11ccccc%11c%10c9)n8)cccc67)c5c4c3)cc2)cc1 ; c1ccc2cc(-c3nc(-c4cc(-c5ccc(-c6ccc7oc8ccccc8c7c6)c6oc7ccccc7c56)c5ccccc5c4)nc(-c4ccc5oc6ccccc6c5c4)n3)ccc2c1]

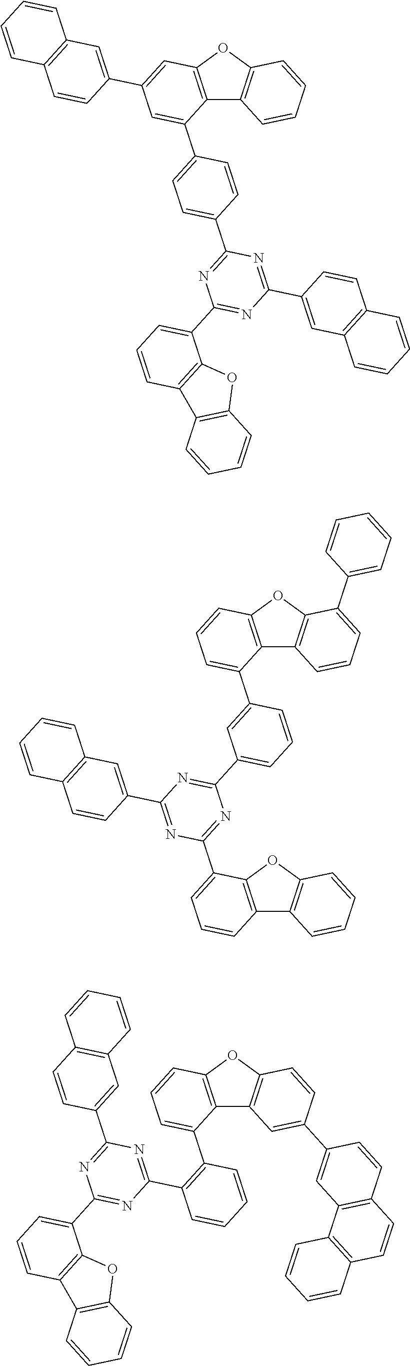 [SMILES: c1ccc2cc(-c3cc(-c4ccc(-c5nc(-c6ccc7ccccc7c6)nc(-c6cccc7c6oc6ccccc67)n5)cc4)c4c(c3)oc3ccccc34)ccc2c1 ; c1ccc(-c2cccc3c2oc2cccc(-c4cccc(-c5nc(-c6ccc7ccccc7c6)nc(-c6cccc7c6oc6ccccc67)n5)c4)c23)cc1 ; c1ccc(-c2cccc3oc4ccc(-c5ccc6ccc7ccccc7c6c5)cc4c23)c(-c2nc(-c3ccc4ccccc4c3)nc(-c3cccc4c3oc3ccccc34)n2)c1]

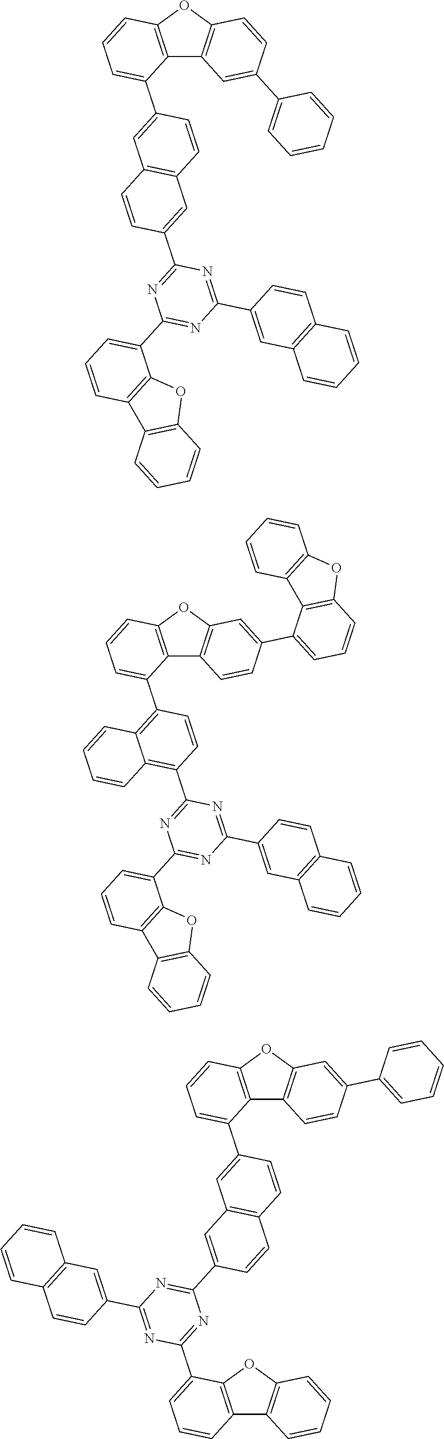 [SMILES: c1ccc(-c2ccc3oc4cccc(-c5ccc6cc(-c7nc(-c8ccc9ccccc9c8)nc(-c8cccc9c8oc8ccccc89)n7)ccc6c5)c4c3c2)cc1 ; c1ccc2cc(-c3nc(-c4ccc(-c5cccc6oc7cc(-c8cccc9oc%10ccccc%10c89)ccc7c56)c5ccccc45)nc(-c4cccc5c4oc4ccccc45)n3)ccc2c1 ; c1ccc(-c2ccc3c(c2)oc2cccc(-c4ccc5ccc(-c6nc(-c7ccc8ccccc8c7)nc(-c7cccc8c7oc7ccccc78)n6)cc5c4)c23)cc1]

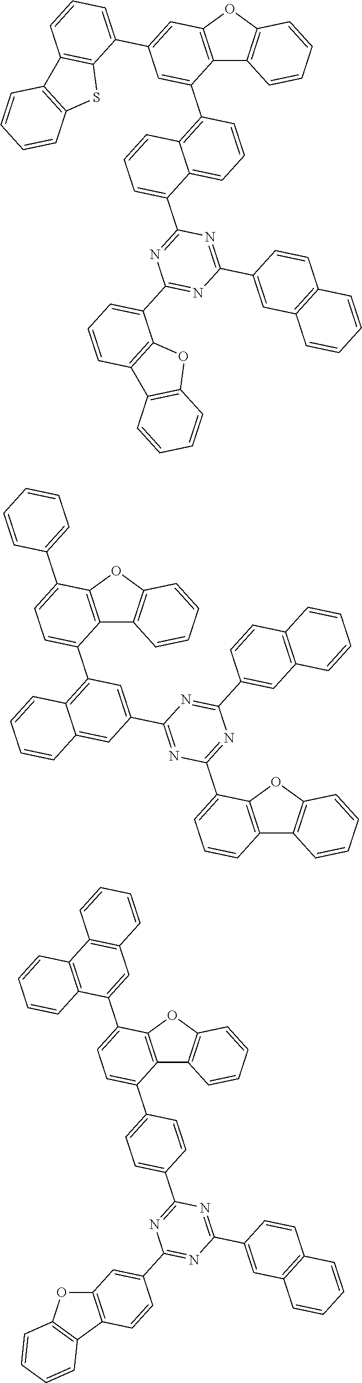 [SMILES: c1ccc2cc(-c3nc(-c4cccc5c(-c6cc(-c7cccc8c7sc7ccccc78)cc7oc8ccccc8c67)cccc45)nc(-c4cccc5c4oc4ccccc45)n3)ccc2c1 ; c1ccc(-c2ccc(-c3cc(-c4nc(-c5ccc6ccccc6c5)nc(-c5cccc6c5oc5ccccc56)n4)cc4ccccc34)c3c2oc2ccccc23)cc1 ; c1ccc2cc(-c3nc(-c4ccc(-c5ccc(-c6cc7ccccc7c7ccccc67)c6oc7ccccc7c56)cc4)nc(-c4ccc5c(c4)oc4ccccc45)n3)ccc2c1]

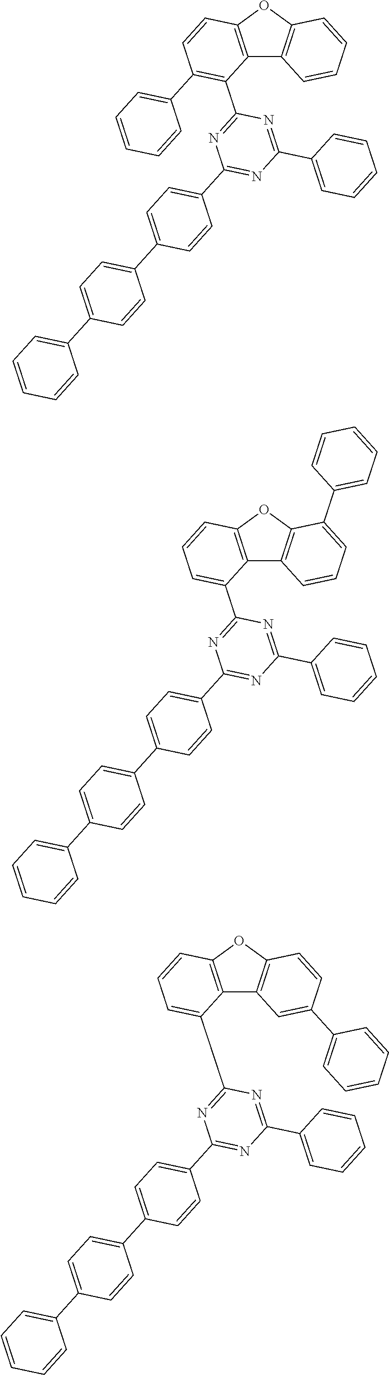 [SMILES: c1ccc(-c2ccc(-c3ccc(-c4nc(-c5ccccc5)nc(-c5c(-c6ccccc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccccc8)cccc7c56)n4)cc3)cc2)cc1 ; c1ccc(-c2ccc(-c3ccc(-c4nc(-c5ccccc5)nc(-c5cccc6oc7ccc(-c8ccccc8)cc7c56)n4)cc3)cc2)cc1]

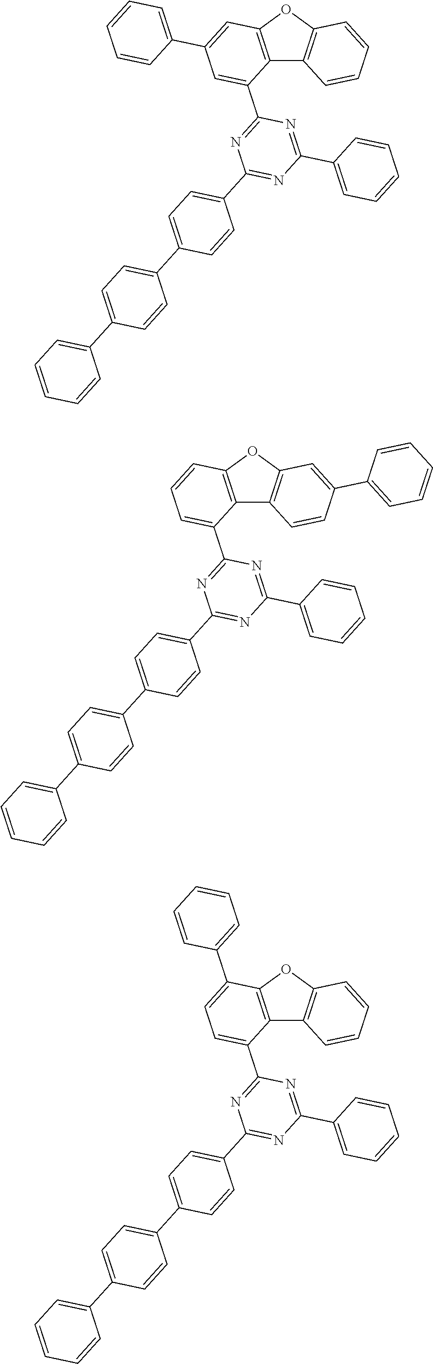 [SMILES: c1ccc(-c2ccc(-c3ccc(-c4nc(-c5ccccc5)nc(-c5cc(-c6ccccc6)cc6oc7ccccc7c56)n4)cc3)cc2)cc1 ; c1ccc(-c2ccc(-c3ccc(-c4nc(-c5ccccc5)nc(-c5cccc6oc7cc(-c8ccccc8)ccc7c56)n4)cc3)cc2)cc1 ; c1ccc(-c2ccc(-c3ccc(-c4nc(-c5ccccc5)nc(-c5ccc(-c6ccccc6)c6oc7ccccc7c56)n4)cc3)cc2)cc1]

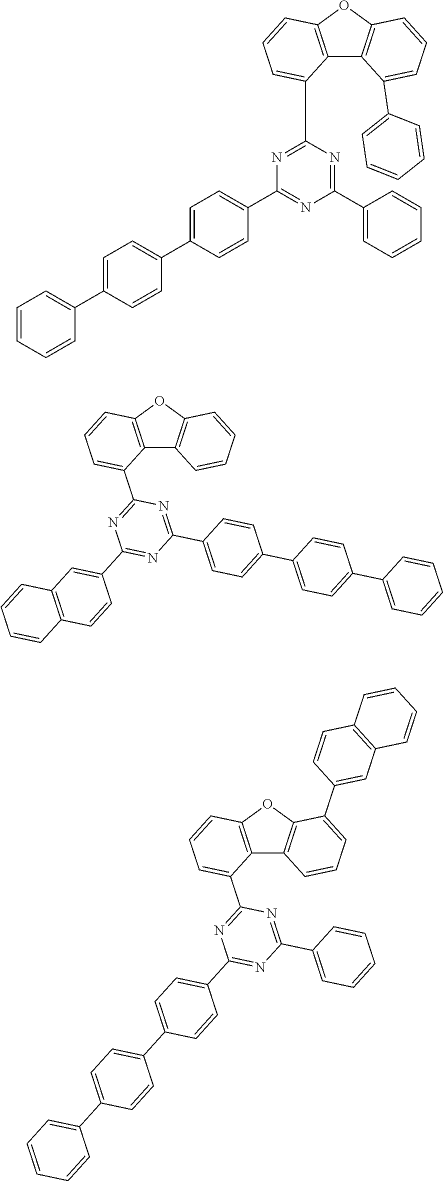 [SMILES: c1ccc(-c2ccc(-c3ccc(-c4nc(-c5ccccc5)nc(-c5cccc6oc7cccc(-c8ccccc8)c7c56)n4)cc3)cc2)cc1 ; c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3ccc(-c4nc(-c5ccccc5)nc(-c5cccc6oc7c(-c8ccc9ccccc9c8)cccc7c56)n4)cc3)cc2)cc1]

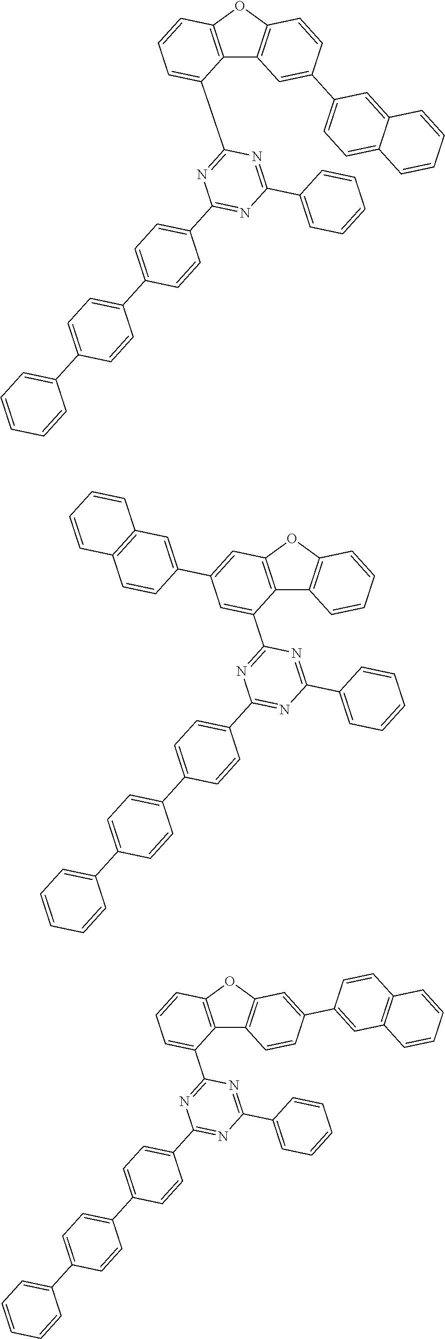 [SMILES: c1ccc(-c2ccc(-c3ccc(-c4nc(-c5ccccc5)nc(-c5cccc6oc7ccc(-c8ccc9ccccc9c8)cc7c56)n4)cc3)cc2)cc1 ; c1ccc(-c2ccc(-c3ccc(-c4nc(-c5ccccc5)nc(-c5cc(-c6ccc7ccccc7c6)cc6oc7ccccc7c56)n4)cc3)cc2)cc1 ; c1ccc(-c2ccc(-c3ccc(-c4nc(-c5ccccc5)nc(-c5cccc6oc7cc(-c8ccc9ccccc9c8)ccc7c56)n4)cc3)cc2)cc1]

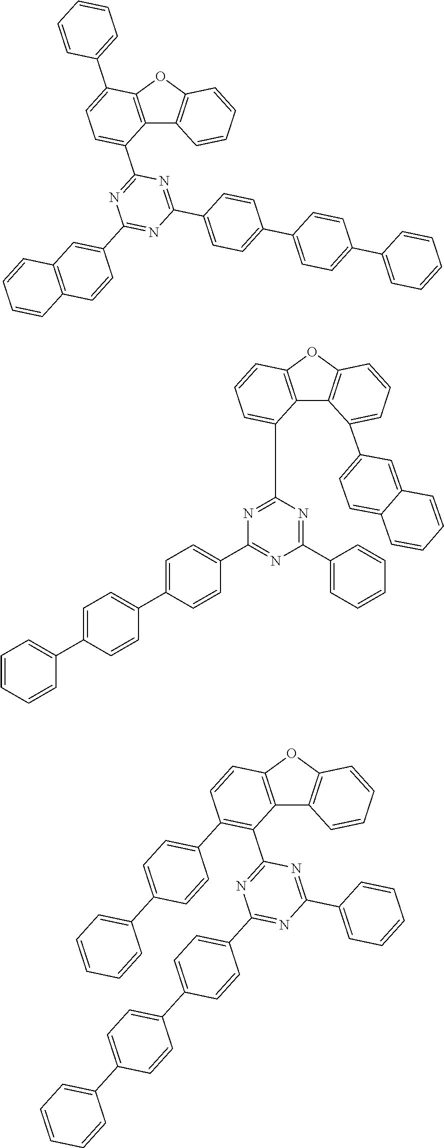 [SMILES: c1ccc(-c2ccc(-c3ccc(-c4nc(-c5ccc6ccccc6c5)nc(-c5ccc(-c6ccccc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9ccccc9c8)c7c56)n4)cc3)cc2)cc1 ; c1ccc(-c2ccc(-c3ccc(-c4nc(-c5ccccc5)nc(-c5c(-c6ccc(-c7ccccc7)cc6)ccc6oc7ccccc7c56)n4)cc3)cc2)cc1]

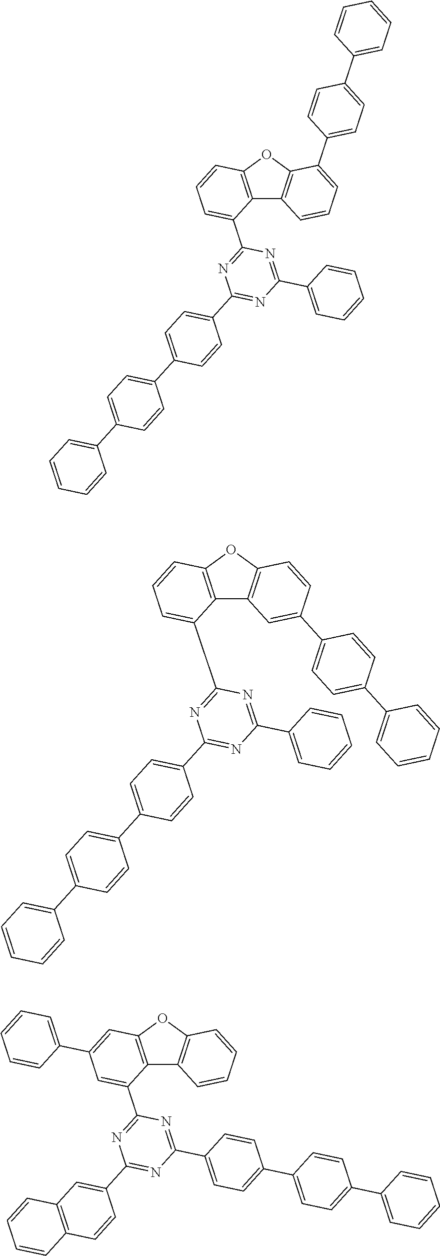 [SMILES: c1ccc(-c2ccc(-c3ccc(-c4nc(-c5ccccc5)nc(-c5cccc6oc7c(-c8ccc(-c9ccccc9)cc8)cccc7c56)n4)cc3)cc2)cc1 ; c1ccc(-c2ccc(-c3ccc(-c4nc(-c5ccccc5)nc(-c5cccc6oc7ccc(-c8ccc(-c9ccccc9)cc8)cc7c56)n4)cc3)cc2)cc1 ; c1ccc(-c2ccc(-c3ccc(-c4nc(-c5ccc6ccccc6c5)nc(-c5cc(-c6ccccc6)cc6oc7ccccc7c56)n4)cc3)cc2)cc1]

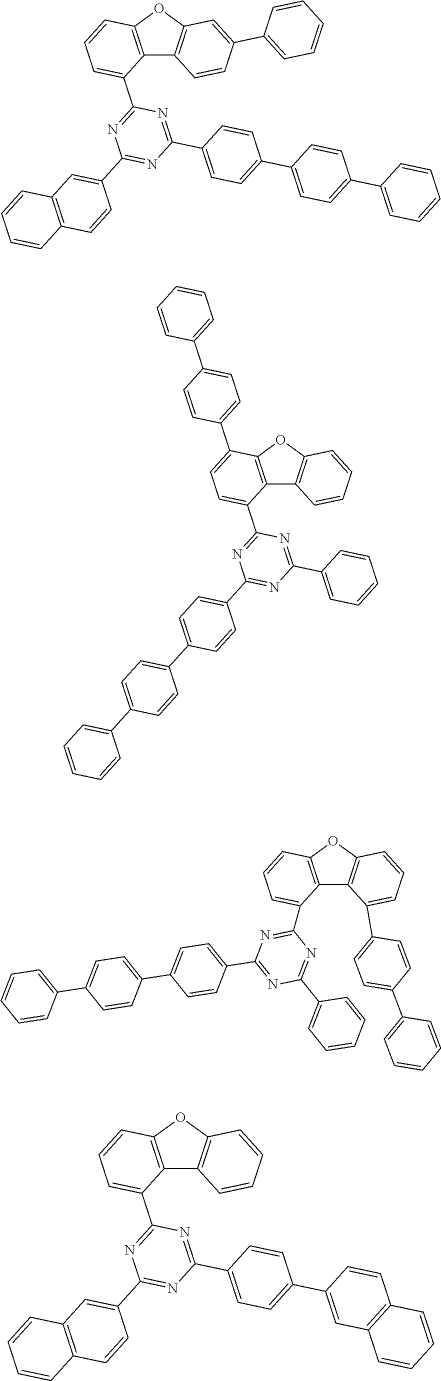 [SMILES: c1ccc(-c2ccc(-c3ccc(-c4nc(-c5ccc6ccccc6c5)nc(-c5cccc6oc7cc(-c8ccccc8)ccc7c56)n4)cc3)cc2)cc1 ; c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1 ; c1ccc2cc(-c3ccc(-c4nc(-c5ccc6ccccc6c5)nc(-c5cccc6oc7ccccc7c56)n4)cc3)ccc2c1]

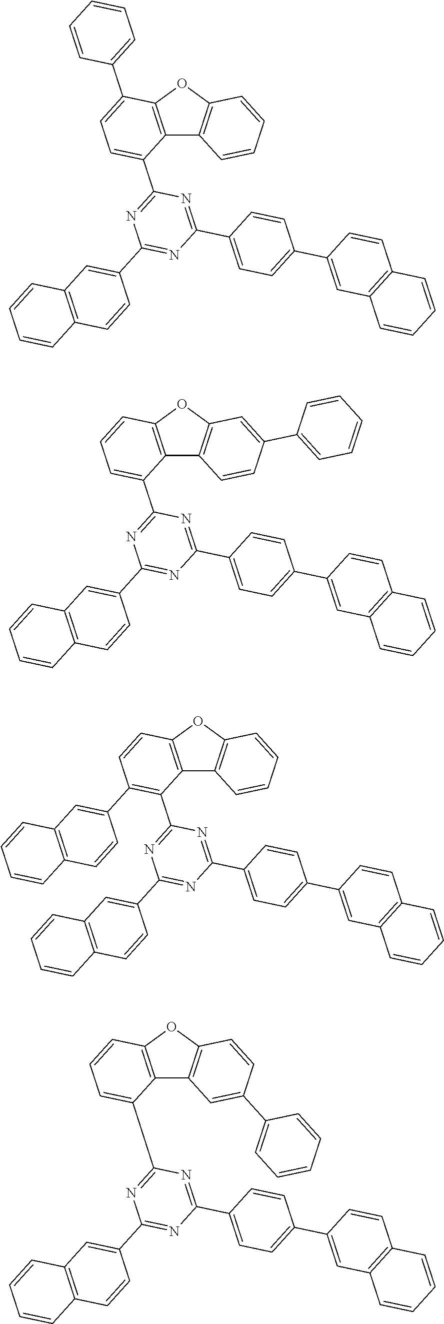 [SMILES: c1ccc(-c2ccc(-c3nc(-c4ccc(-c5ccc6ccccc6c5)cc4)nc(-c4ccc5ccccc5c4)n3)c3c2oc2ccccc23)cc1 ; c1ccc(-c2ccc3c(c2)oc2cccc(-c4nc(-c5ccc(-c6ccc7ccccc7c6)cc5)nc(-c5ccc6ccccc6c5)n4)c23)cc1 ; c1ccc2cc(-c3ccc(-c4nc(-c5ccc6ccccc6c5)nc(-c5c(-c6ccc7ccccc7c6)ccc6oc7ccccc7c56)n4)cc3)ccc2c1 ; c1ccc(-c2ccc3oc4cccc(-c5nc(-c6ccc(-c7ccc8ccccc8c7)cc6)nc(-c6ccc7ccccc7c6)n5)c4c3c2)cc1]

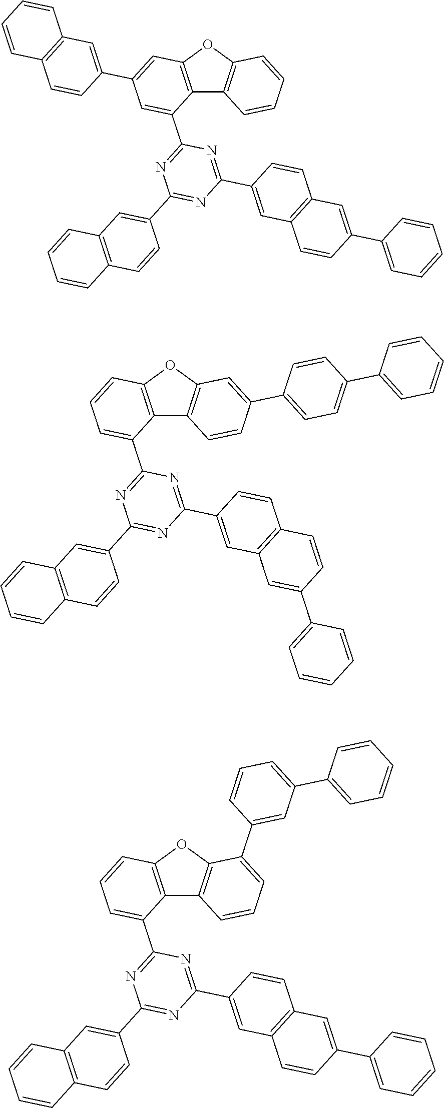 [SMILES: c1ccc(-c2ccc3cc(-c4nc(-c5ccc6ccccc6c5)nc(-c5cc(-c6ccc7ccccc7c6)cc6oc7ccccc7c56)n4)ccc3c2)cc1 ; c1ccc(-c2ccc(-c3ccc4c(c3)oc3cccc(-c5nc(-c6ccc7ccccc7c6)nc(-c6ccc7ccc(-c8ccccc8)cc7c6)n5)c34)cc2)cc1 ; c1ccc(-c2cccc(-c3cccc4c3oc3cccc(-c5nc(-c6ccc7ccccc7c6)nc(-c6ccc7cc(-c8ccccc8)ccc7c6)n5)c34)c2)cc1]

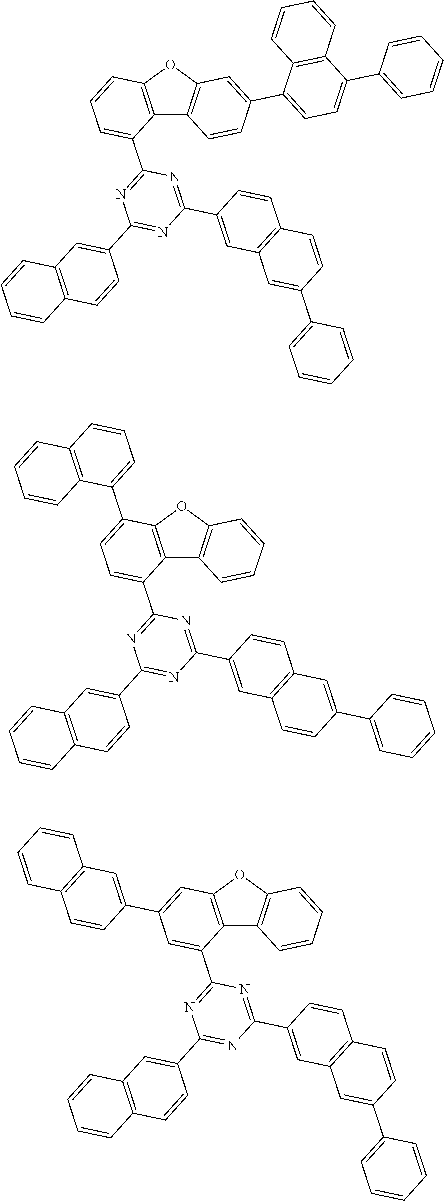 [SMILES: c1ccc(-c2ccc3ccc(-c4nc(-c5ccc6ccccc6c5)nc(-c5cccc6oc7cc(-c8ccc(-c9ccccc9)c9ccccc89)ccc7c56)n4)cc3c2)cc1 ; c1ccc(-c2ccc3cc(-c4nc(-c5ccc6ccccc6c5)nc(-c5ccc(-c6cccc7ccccc67)c6oc7ccccc7c56)n4)ccc3c2)cc1 ; c1ccc(-c2ccc3ccc(-c4nc(-c5ccc6ccccc6c5)nc(-c5cc(-c6ccc7ccccc7c6)cc6oc7ccccc7c56)n4)cc3c2)cc1]

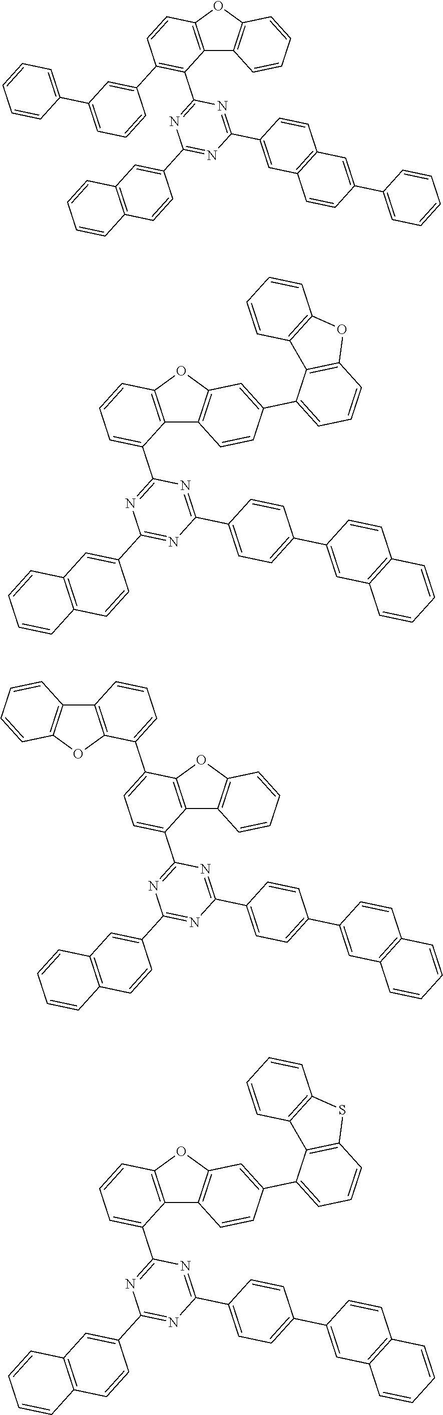 [SMILES: c1ccc(-c2cccc(-c3ccc4oc5ccccc5c4c3-c3nc(-c4ccc5ccccc5c4)nc(-c4ccc5cc(-c6ccccc6)ccc5c4)n3)c2)cc1 ; c1ccc2cc(-c3ccc(-c4nc(-c5ccc6ccccc6c5)nc(-c5cccc6oc7cc(-c8cccc9oc%10ccccc%10c89)ccc7c56)n4)cc3)ccc2c1 ; c1ccc2cc(-c3ccc(-c4nc(-c5ccc6ccccc6c5)nc(-c5ccc(-c6cccc7c6oc6ccccc67)c6oc7ccccc7c56)n4)cc3)ccc2c1 ; c1ccc2cc(-c3ccc(-c4nc(-c5ccc6ccccc6c5)nc(-c5cccc6oc7cc(-c8cccc9sc%10ccccc%10c89)ccc7c56)n4)cc3)ccc2c1]

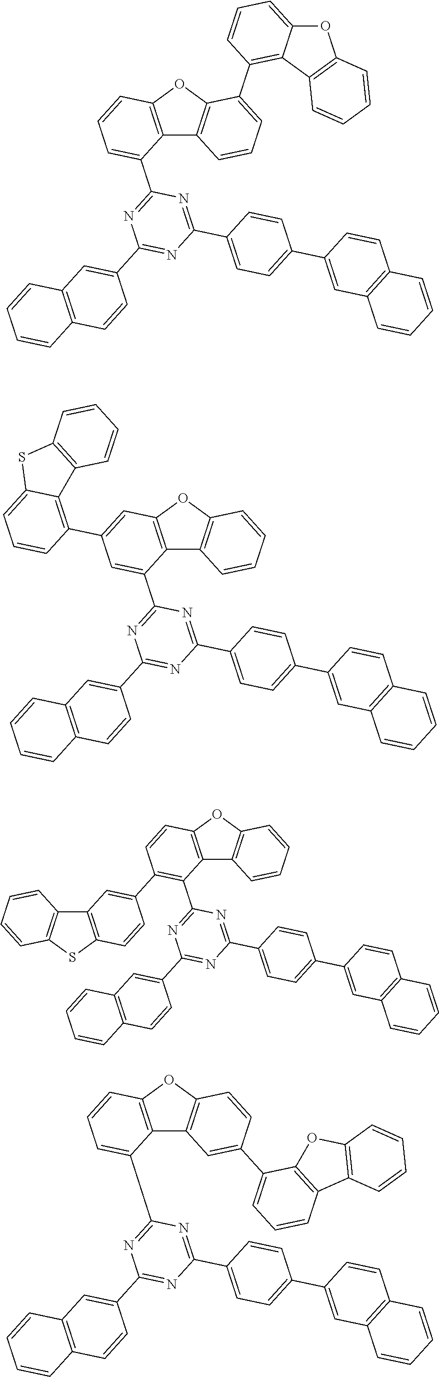 [SMILES: c1ccc2cc(-c3ccc(-c4nc(-c5ccc6ccccc6c5)nc(-c5cccc6oc7c(-c8cccc9oc%10ccccc%10c89)cccc7c56)n4)cc3)ccc2c1 ; c1ccc2cc(-c3ccc(-c4nc(-c5ccc6ccccc6c5)nc(-c5cc(-c6cccc7sc8ccccc8c67)cc6oc7ccccc7c56)n4)cc3)ccc2c1 ; c1ccc2cc(-c3ccc(-c4nc(-c5ccc6ccccc6c5)nc(-c5c(-c6ccc7sc8ccccc8c7c6)ccc6oc7ccccc7c56)n4)cc3)ccc2c1 ; c1ccc2cc(-c3ccc(-c4nc(-c5ccc6ccccc6c5)nc(-c5cccc6oc7ccc(-c8cccc9c8oc8ccccc89)cc7c56)n4)cc3)ccc2c1]

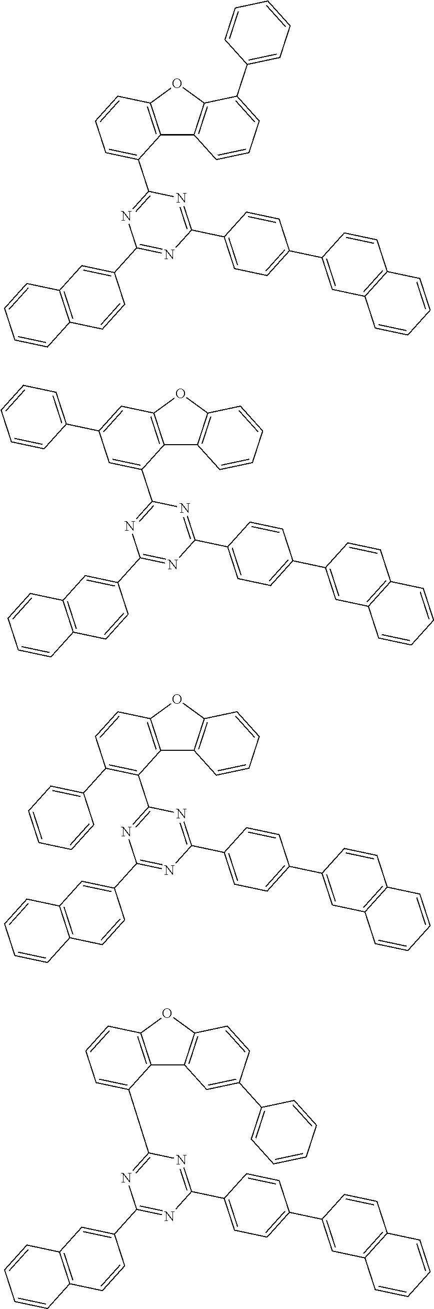

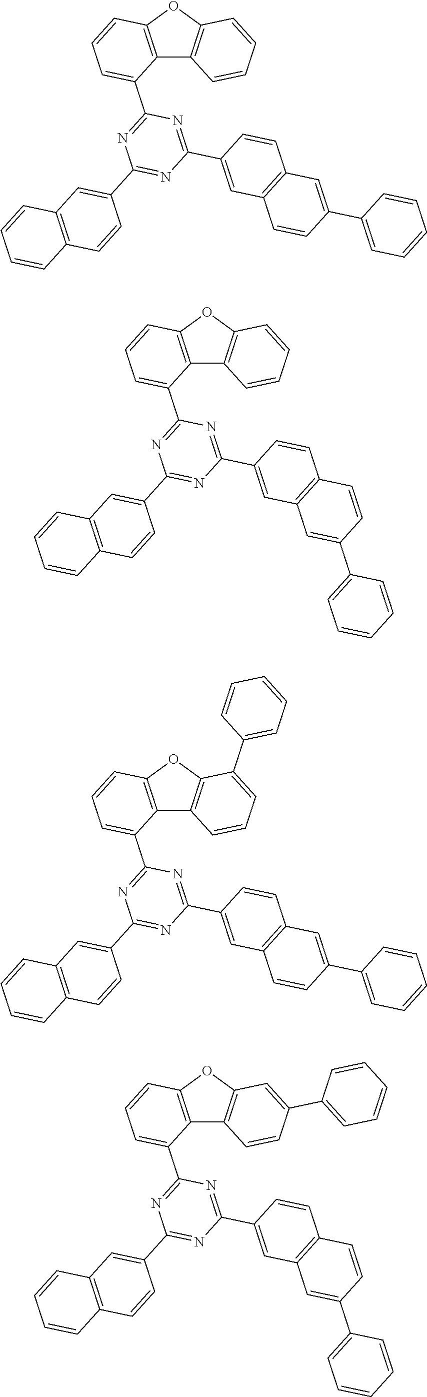 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc3ccc(-c4nc(-c5ccc6ccccc6c5)nc(-c5cccc6oc7ccccc7c56)n4)cc3c2)cc1 ; c1ccc(-c2ccc3cc(-c4nc(-c5ccc6ccccc6c5)nc(-c5cccc6oc7c(-c8ccccc8)cccc7c56)n4)ccc3c2)cc1 ; c1ccc(-c2ccc3ccc(-c4nc(-c5ccc6ccccc6c5)nc(-c5cccc6oc7cc(-c8ccccc8)ccc7c56)n4)cc3c2)cc1]

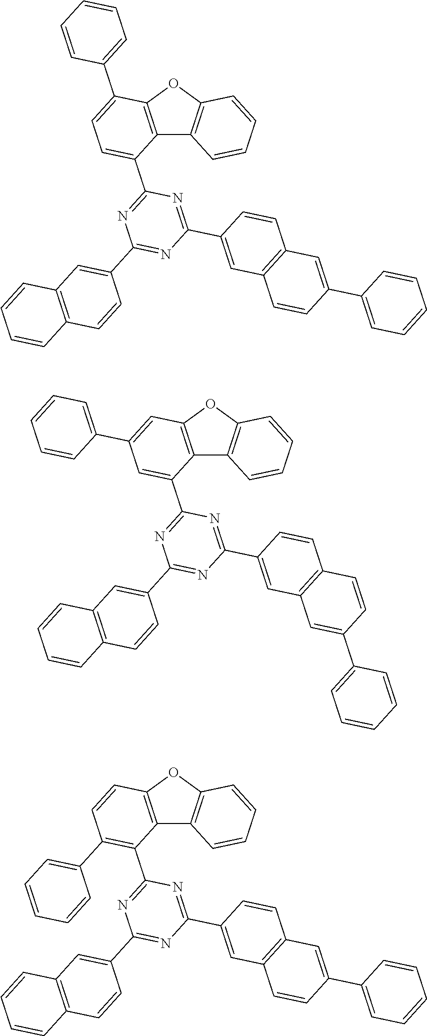 [SMILES: c1ccc(-c2ccc3cc(-c4nc(-c5ccc6ccccc6c5)nc(-c5ccc(-c6ccccc6)c6oc7ccccc7c56)n4)ccc3c2)cc1 ; c1ccc(-c2ccc3ccc(-c4nc(-c5ccc6ccccc6c5)nc(-c5cc(-c6ccccc6)cc6oc7ccccc7c56)n4)cc3c2)cc1 ; c1ccc(-c2ccc3cc(-c4nc(-c5ccc6ccccc6c5)nc(-c5c(-c6ccccc6)ccc6oc7ccccc7c56)n4)ccc3c2)cc1]

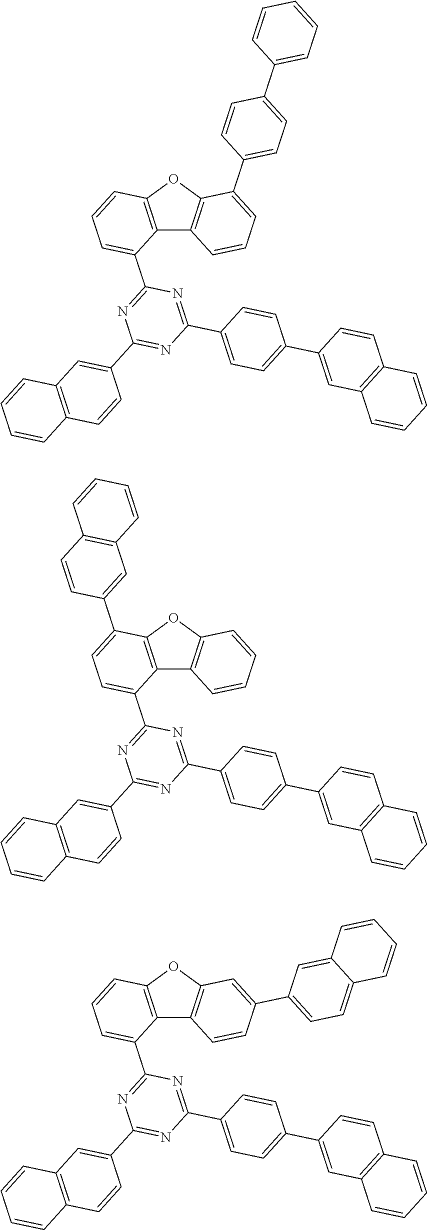 [SMILES: c1ccc(-c2ccc(-c3cccc4c3oc3cccc(-c5nc(-c6ccc(-c7ccc8ccccc8c7)cc6)nc(-c6ccc7ccccc7c6)n5)c34)cc2)cc1 ; c1ccc2cc(-c3ccc(-c4nc(-c5ccc6ccccc6c5)nc(-c5ccc(-c6ccc7ccccc7c6)c6oc7ccccc7c56)n4)cc3)ccc2c1 ; c1ccc2cc(-c3ccc(-c4nc(-c5ccc6ccccc6c5)nc(-c5cccc6oc7cc(-c8ccc9ccccc9c8)ccc7c56)n4)cc3)ccc2c1]

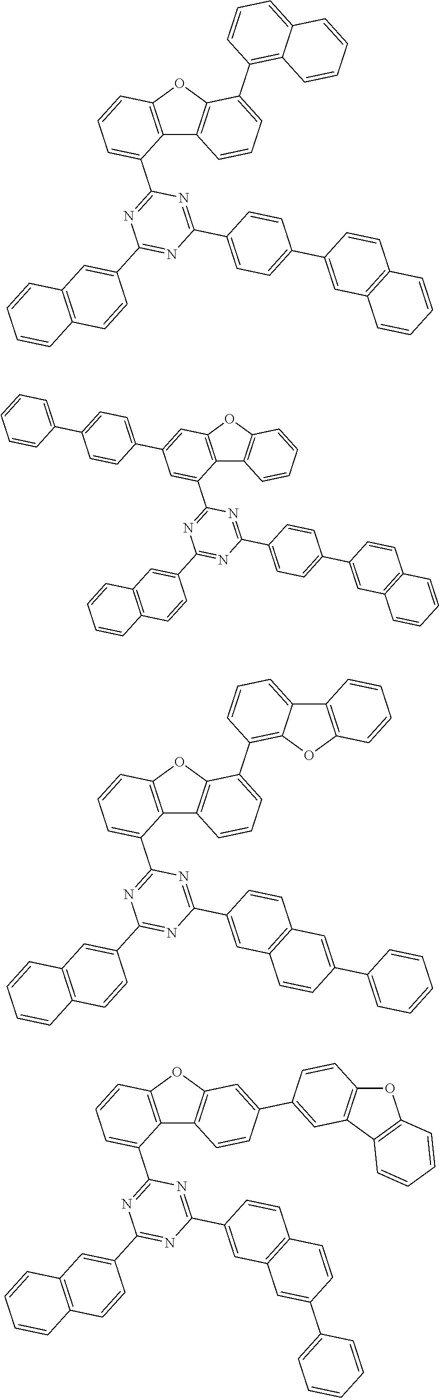 [SMILES: c1ccc2cc(-c3ccc(-c4nc(-c5ccc6ccccc6c5)nc(-c5cccc6oc7c(-c8cccc9ccccc89)cccc7c56)n4)cc3)ccc2c1 ; c1ccc(-c2ccc(-c3cc(-c4nc(-c5ccc(-c6ccc7ccccc7c6)cc5)nc(-c5ccc6ccccc6c5)n4)c4c(c3)oc3ccccc34)cc2)cc1 ; c1ccc(-c2ccc3cc(-c4nc(-c5ccc6ccccc6c5)nc(-c5cccc6oc7c(-c8cccc9c8oc8ccccc89)cccc7c56)n4)ccc3c2)cc1 ; c1ccc(-c2ccc3ccc(-c4nc(-c5ccc6ccccc6c5)nc(-c5cccc6oc7cc(-c8ccc9oc%10ccccc%10c9c8)ccc7c56)n4)cc3c2)cc1]

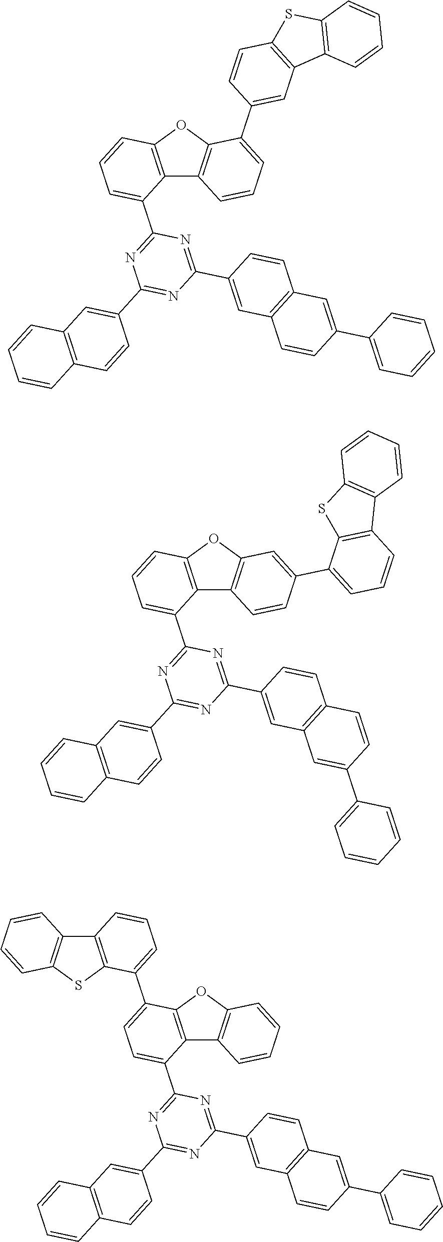 [SMILES: c1ccc(-c2ccc3cc(-c4nc(-c5ccc6ccccc6c5)nc(-c5cccc6oc7c(-c8ccc9sc%10ccccc%10c9c8)cccc7c56)n4)ccc3c2)cc1 ; c1ccc(-c2ccc3ccc(-c4nc(-c5ccc6ccccc6c5)nc(-c5cccc6oc7cc(-c8cccc9c8sc8ccccc89)ccc7c56)n4)cc3c2)cc1 ; c1ccc(-c2ccc3cc(-c4nc(-c5ccc6ccccc6c5)nc(-c5ccc(-c6cccc7c6sc6ccccc67)c6oc7ccccc7c56)n4)ccc3c2)cc1]

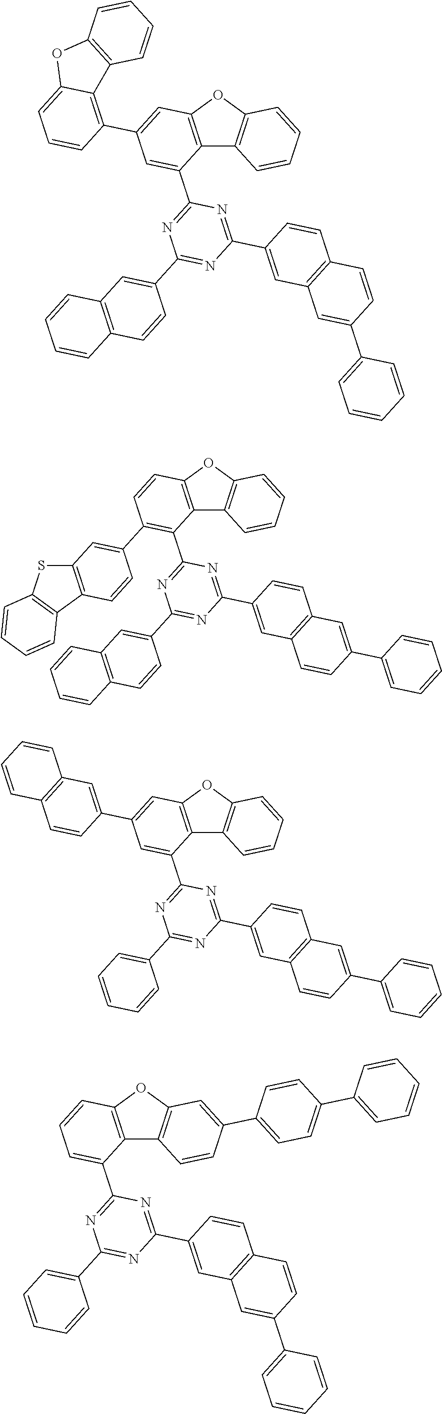 [SMILES: c1ccc(-c2ccc3ccc(-c4nc(-c5ccc6ccccc6c5)nc(-c5cc(-c6cccc7oc8ccccc8c67)cc6oc7ccccc7c56)n4)cc3c2)cc1 ; c1ccc(-c2ccc3cc(-c4nc(-c5ccc6ccccc6c5)nc(-c5c(-c6ccc7c(c6)sc6ccccc67)ccc6oc7ccccc7c56)n4)ccc3c2)cc1 ; c1ccc(-c2ccc3cc(-c4nc(-c5ccccc5)nc(-c5cc(-c6ccc7ccccc7c6)cc6oc7ccccc7c56)n4)ccc3c2)cc1 ; c1ccc(-c2ccc(-c3ccc4c(c3)oc3cccc(-c5nc(-c6ccccc6)nc(-c6ccc7ccc(-c8ccccc8)cc7c6)n5)c34)cc2)cc1]

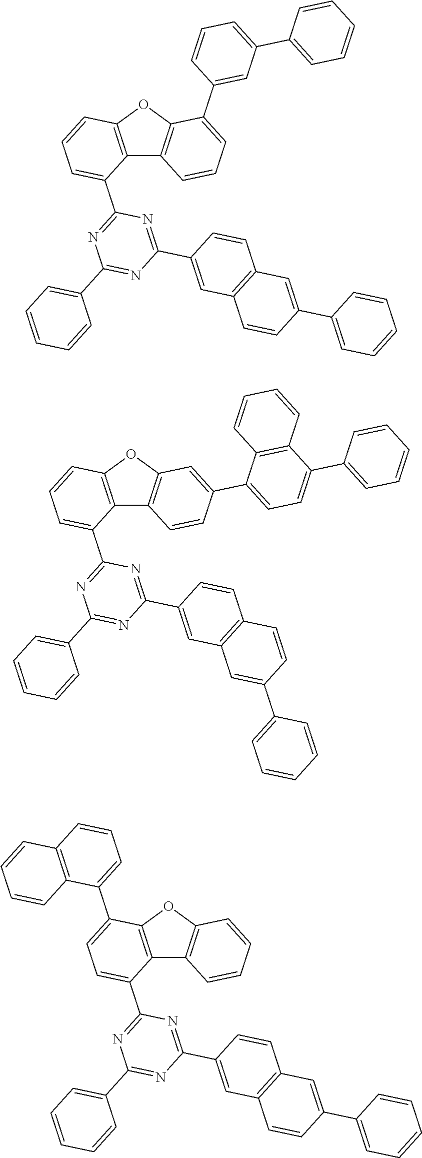 [SMILES: c1ccc(-c2cccc(-c3cccc4c3oc3cccc(-c5nc(-c6ccccc6)nc(-c6ccc7cc(-c8ccccc8)ccc7c6)n5)c34)c2)cc1 ; c1ccc(-c2ccc3ccc(-c4nc(-c5ccccc5)nc(-c5cccc6oc7cc(-c8ccc(-c9ccccc9)c9ccccc89)ccc7c56)n4)cc3c2)cc1 ; c1ccc(-c2ccc3cc(-c4nc(-c5ccccc5)nc(-c5ccc(-c6cccc7ccccc67)c6oc7ccccc7c56)n4)ccc3c2)cc1]

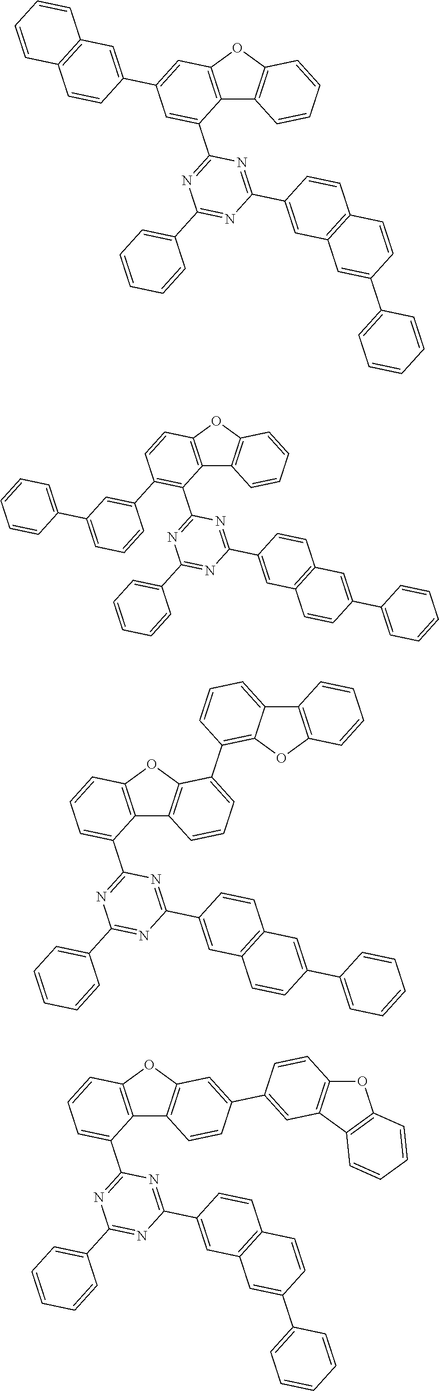 [SMILES: c1ccc(-c2ccc3ccc(-c4nc(-c5ccccc5)nc(-c5cc(-c6ccc7ccccc7c6)cc6oc7ccccc7c56)n4)cc3c2)cc1 ; c1ccc(-c2cccc(-c3ccc4oc5ccccc5c4c3-c3nc(-c4ccccc4)nc(-c4ccc5cc(-c6ccccc6)ccc5c4)n3)c2)cc1 ; c1ccc(-c2ccc3cc(-c4nc(-c5ccccc5)nc(-c5cccc6oc7c(-c8cccc9c8oc8ccccc89)cccc7c56)n4)ccc3c2)cc1 ; c1ccc(-c2ccc3ccc(-c4nc(-c5ccccc5)nc(-c5cccc6oc7cc(-c8ccc9oc%10ccccc%10c9c8)ccc7c56)n4)cc3c2)cc1]

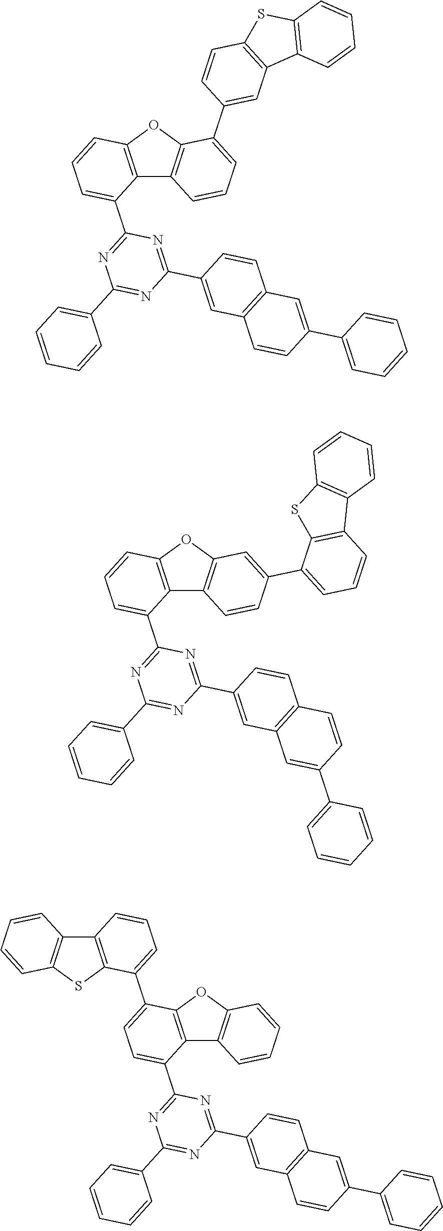 [SMILES: c1ccc(-c2ccc3cc(-c4nc(-c5ccccc5)nc(-c5cccc6oc7c(-c8ccc9sc%10ccccc%10c9c8)cccc7c56)n4)ccc3c2)cc1 ; c1ccc(-c2ccc3ccc(-c4nc(-c5ccccc5)nc(-c5cccc6oc7cc(-c8cccc9c8sc8ccccc89)ccc7c56)n4)cc3c2)cc1 ; c1ccc(-c2ccc3cc(-c4nc(-c5ccccc5)nc(-c5ccc(-c6cccc7c6sc6ccccc67)c6oc7ccccc7c56)n4)ccc3c2)cc1]

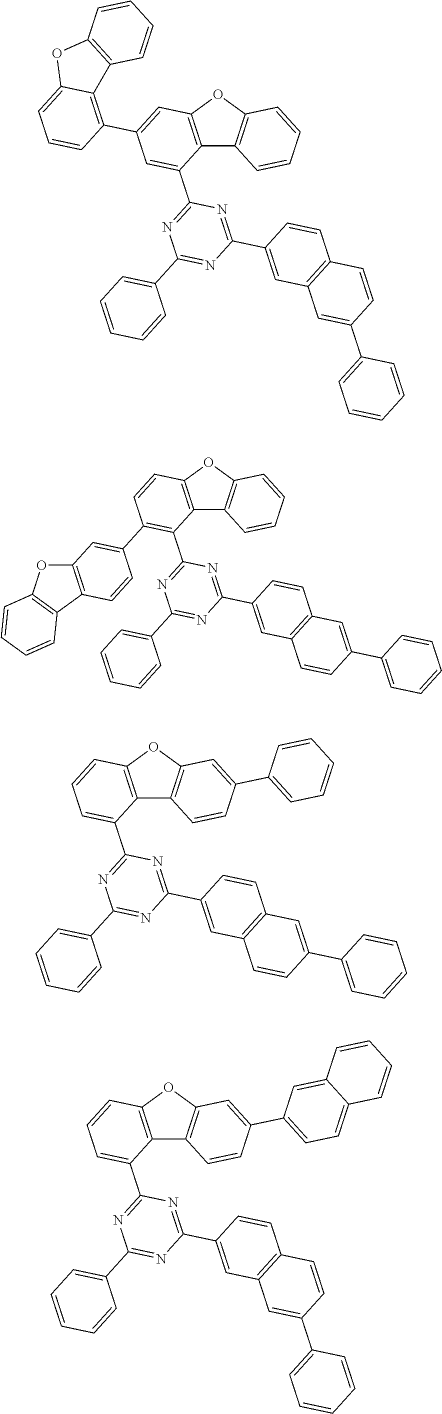

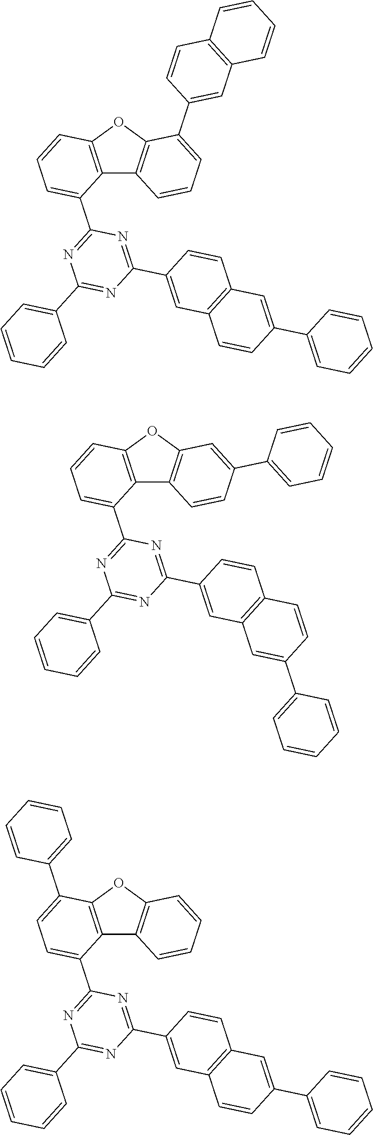 [SMILES: c1ccc(-c2ccc3cc(-c4nc(-c5ccccc5)nc(-c5cccc6oc7c(-c8ccc9ccccc9c8)cccc7c56)n4)ccc3c2)cc1 ; c1ccc(-c2ccc3ccc(-c4nc(-c5ccccc5)nc(-c5cccc6oc7cc(-c8ccccc8)ccc7c56)n4)cc3c2)cc1 ; c1ccc(-c2ccc3cc(-c4nc(-c5ccccc5)nc(-c5ccc(-c6ccccc6)c6oc7ccccc7c56)n4)ccc3c2)cc1]

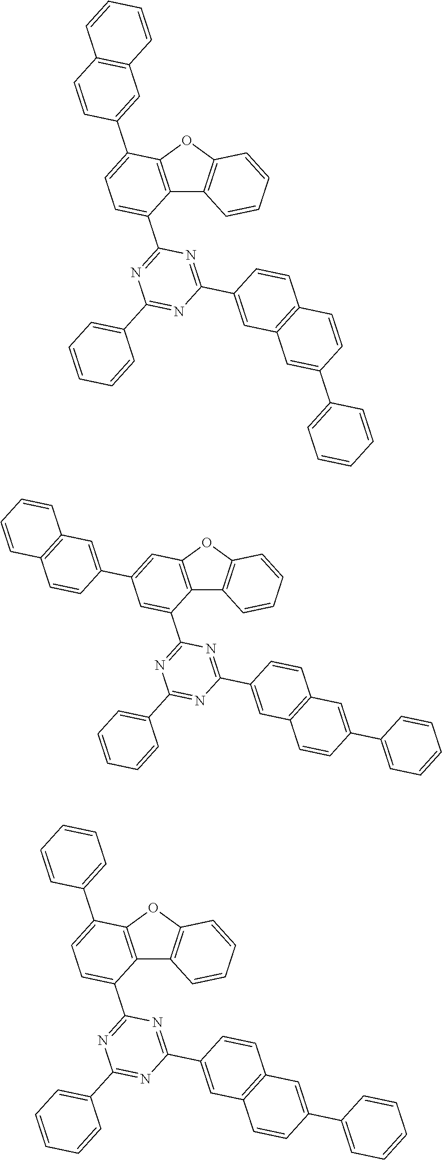 [SMILES: c1ccc(-c2ccc3ccc(-c4nc(-c5ccccc5)nc(-c5ccc(-c6ccc7ccccc7c6)c6oc7ccccc7c56)n4)cc3c2)cc1 ; c1ccc(-c2ccc3cc(-c4nc(-c5ccccc5)nc(-c5cc(-c6ccc7ccccc7c6)cc6oc7ccccc7c56)n4)ccc3c2)cc1 ; c1ccc(-c2ccc3cc(-c4nc(-c5ccccc5)nc(-c5ccc(-c6ccccc6)c6oc7ccccc7c56)n4)ccc3c2)cc1]

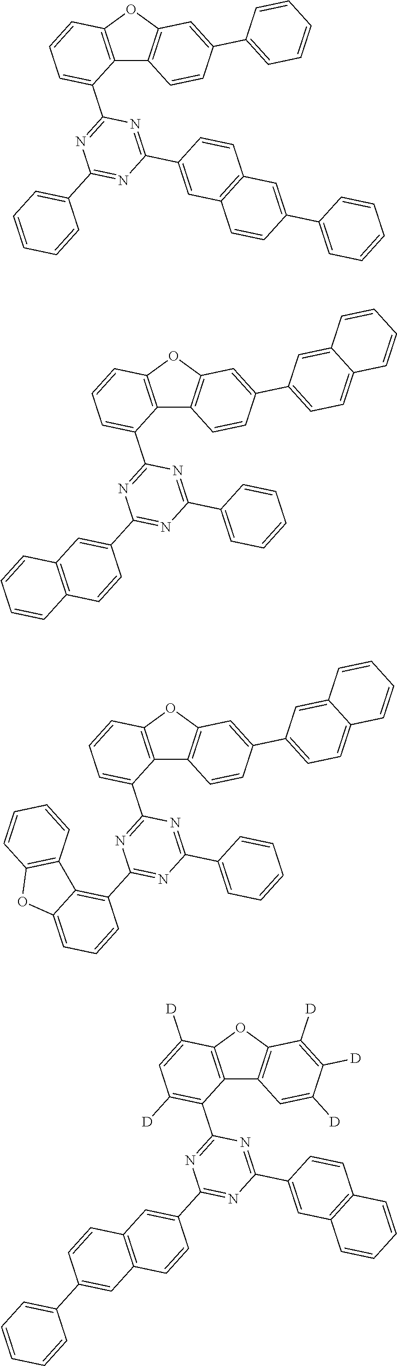 [SMILES: c1ccc(-c2ccc3cc(-c4nc(-c5ccccc5)nc(-c5cccc6oc7cc(-c8ccccc8)ccc7c56)n4)ccc3c2)cc1 ; c1ccc(-c2nc(-c3ccc4ccccc4c3)nc(-c3cccc4oc5cc(-c6ccc7ccccc7c6)ccc5c34)n2)cc1 ; c1ccc(-c2nc(-c3cccc4oc5ccccc5c34)nc(-c3cccc4oc5cc(-c6ccc7ccccc7c6)ccc5c34)n2)cc1 ; [2H]c1cc2c(oc3c([2H])cc([2H])c(-c4nc(-c5ccc6ccccc6c5)nc(-c5ccc6cc(-c7ccccc7)ccc6c5)n4)c32)c([2H])c1[2H]]

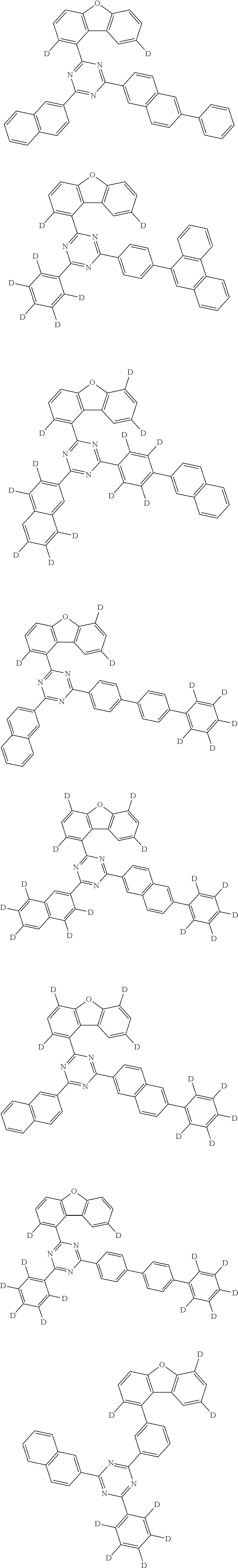

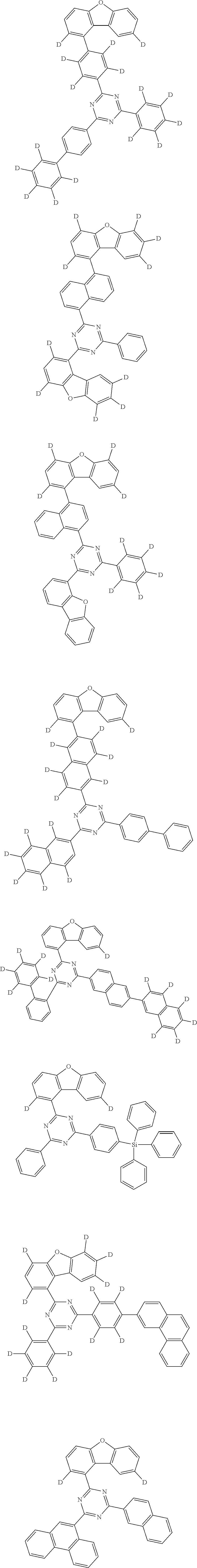

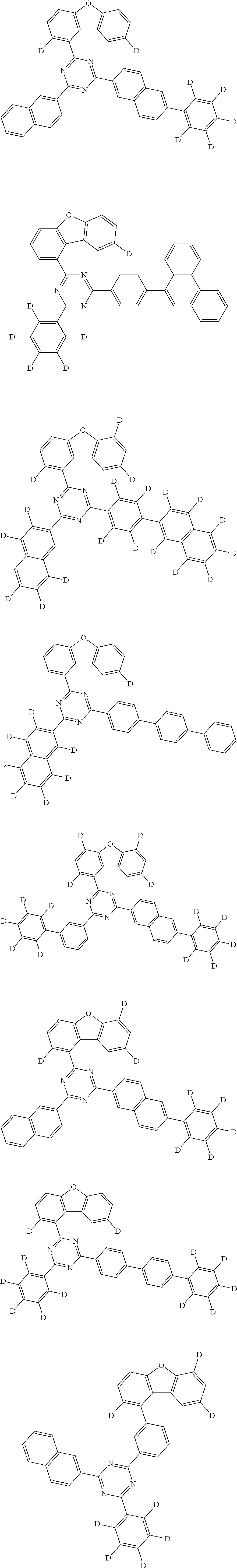

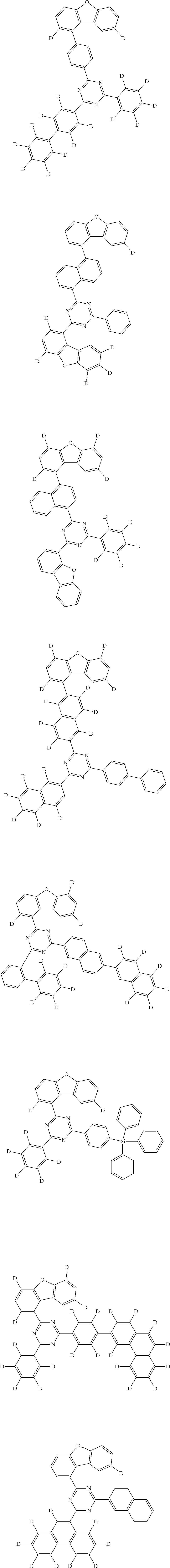

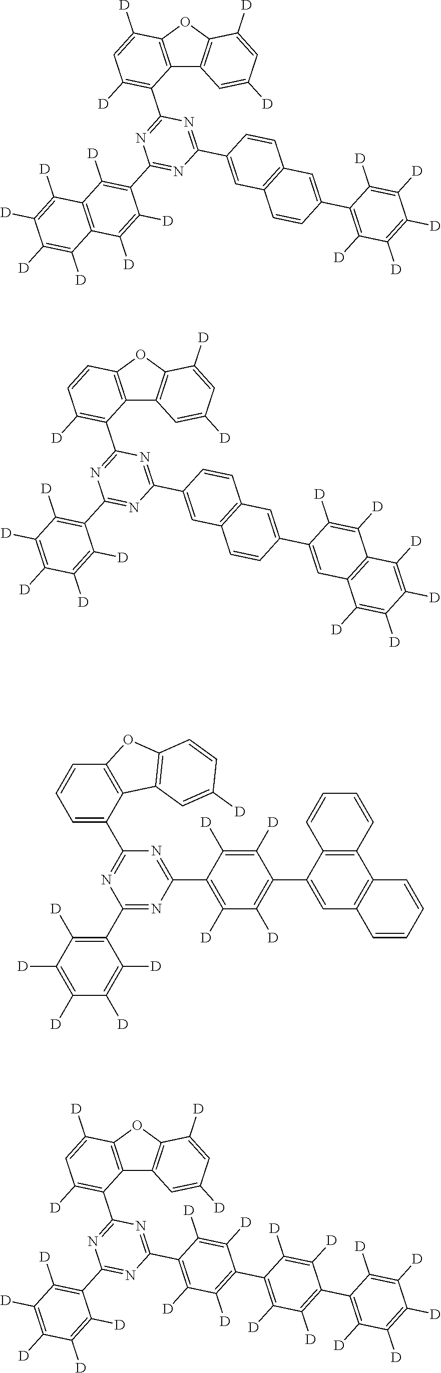

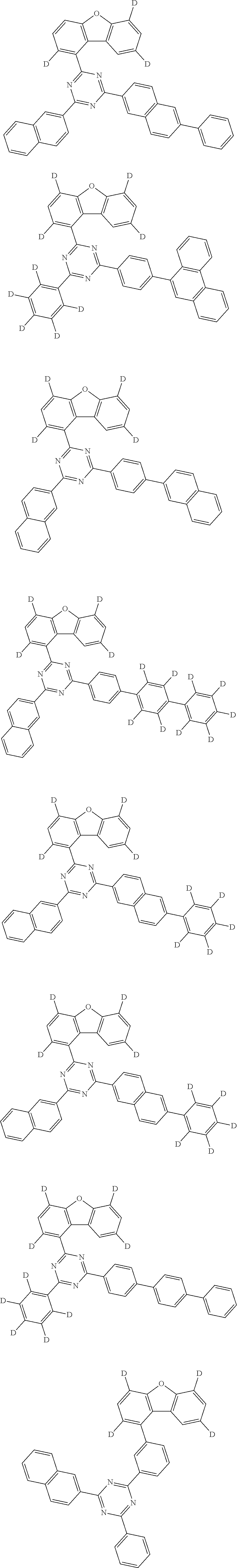

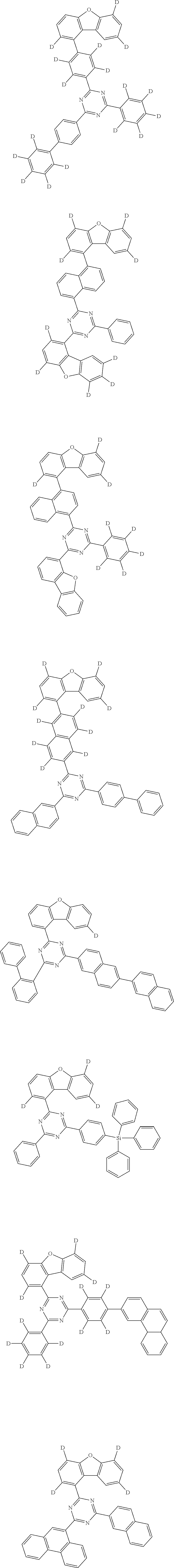

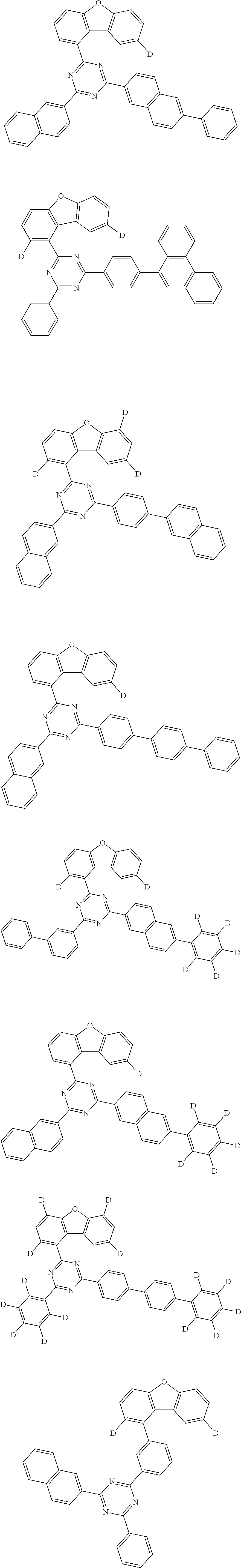

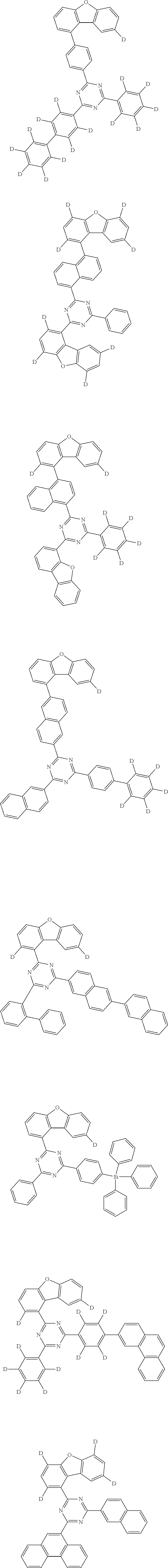

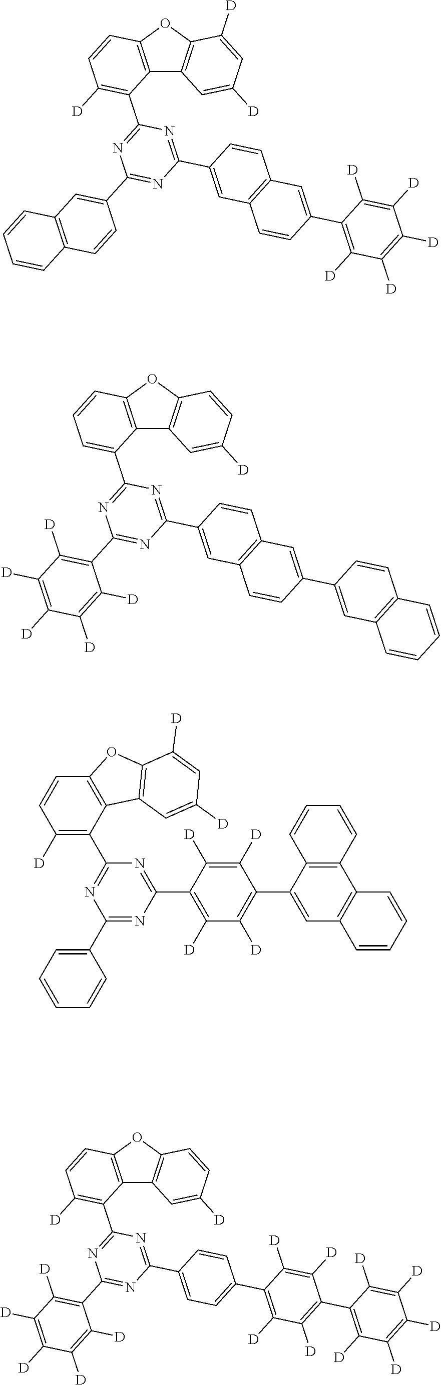

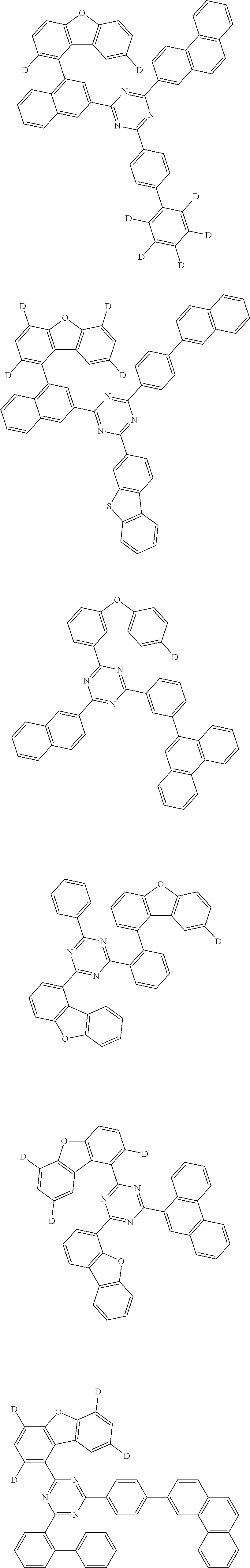

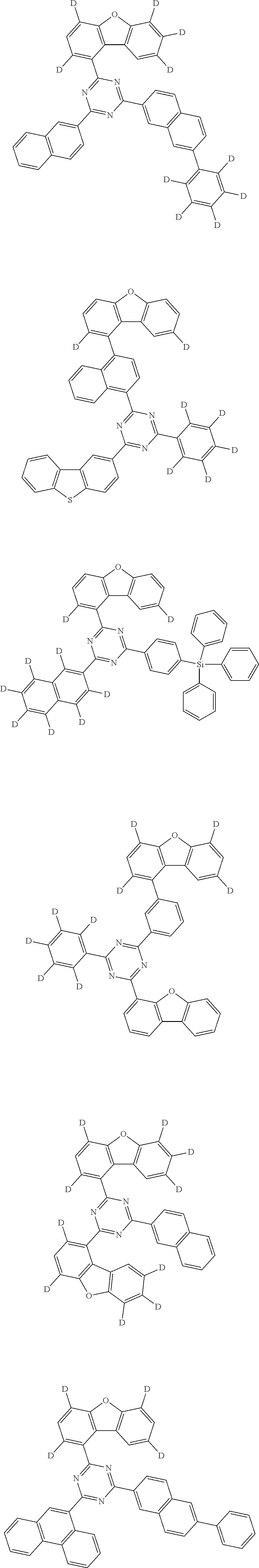

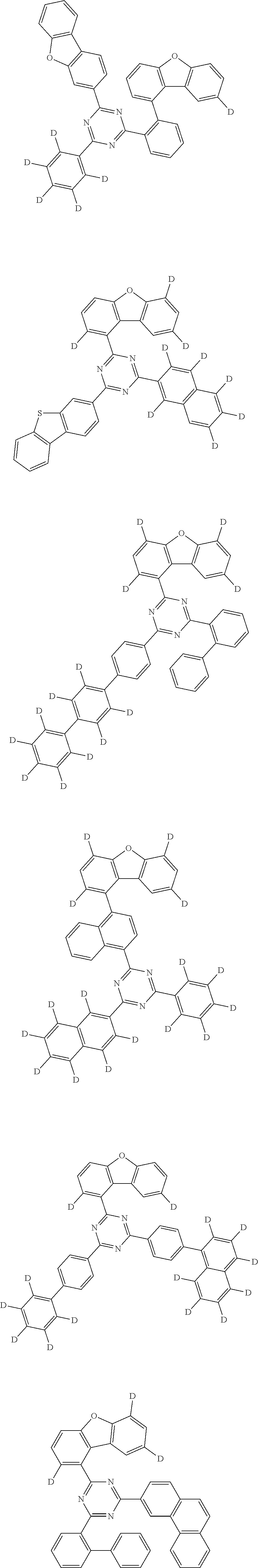

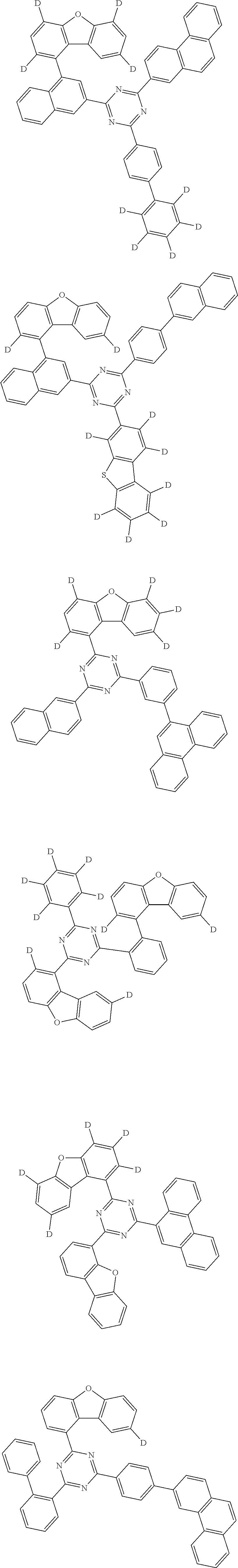

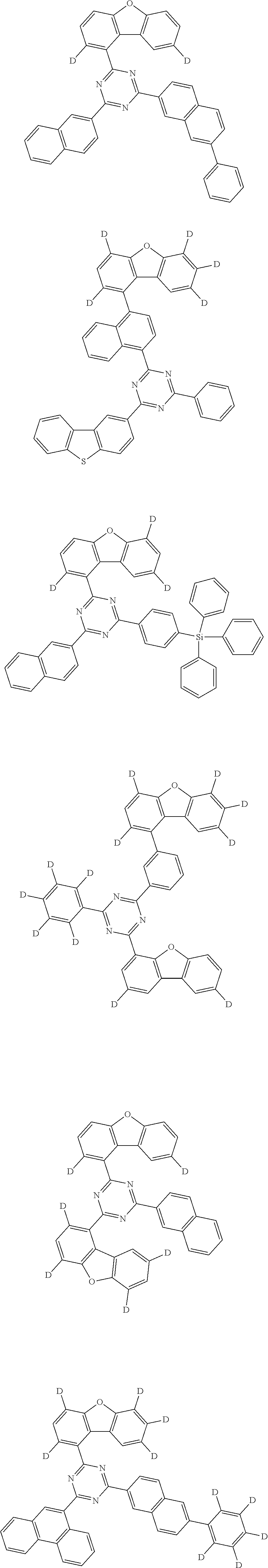

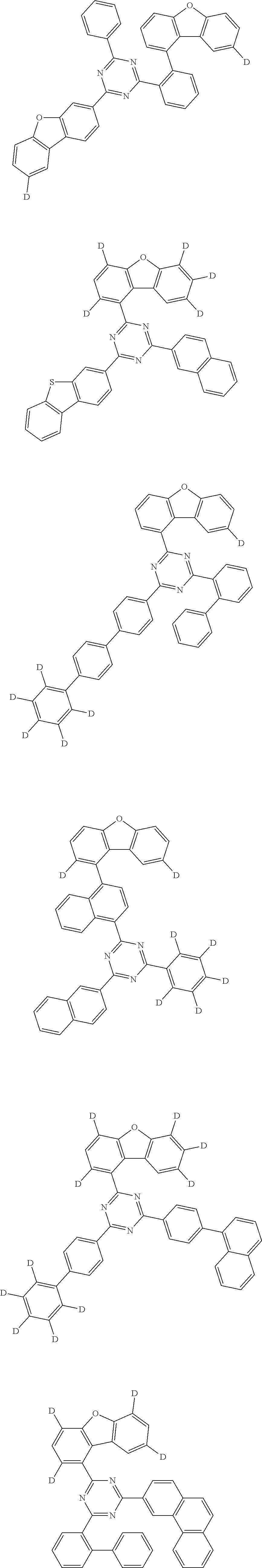

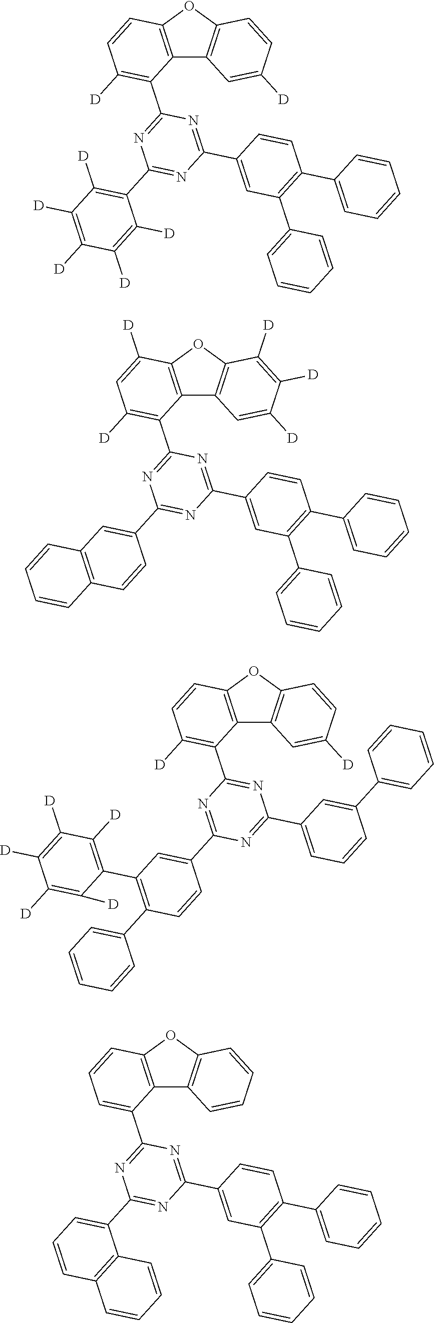

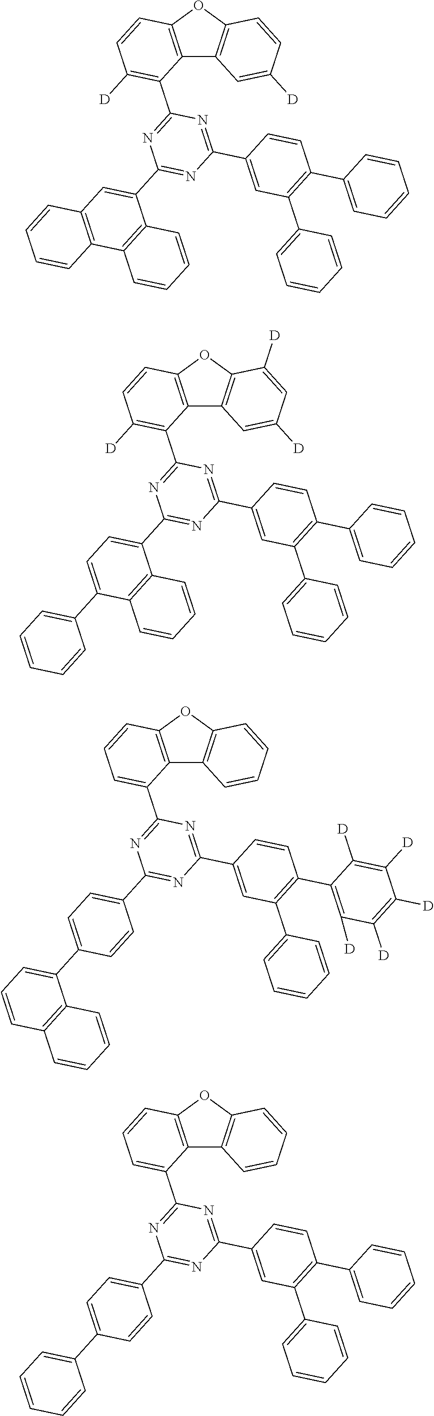 [SMILES: [2H]c1ccc2oc3ccc([2H])c(-c4nc(-c5ccc(-c6ccccc6)c(-c6ccccc6)c5)nc(-c5cc6ccccc6c6ccccc56)n4)c3c2c1 ; [2H]c1cc([2H])c2oc3ccc([2H])c(-c4nc(-c5ccc(-c6ccccc6)c(-c6ccccc6)c5)nc(-c5ccc(-c6ccccc6)c6ccccc56)n4)c3c2c1 ; [2H]c1c([2H])c([2H])c(-c2ccc(-c3nc(-c4ccc(-c5cccc6ccccc56)cc4)nc(-c4cccc5oc6ccccc6c45)n3)cc2-c2ccccc2)c([2H])c1[2H] ; c1ccc(-c2ccc(-c3nc(-c4ccc(-c5ccccc5)c(-c5ccccc5)c4)nc(-c4cccc5oc6ccccc6c45)n3)cc2)cc1]

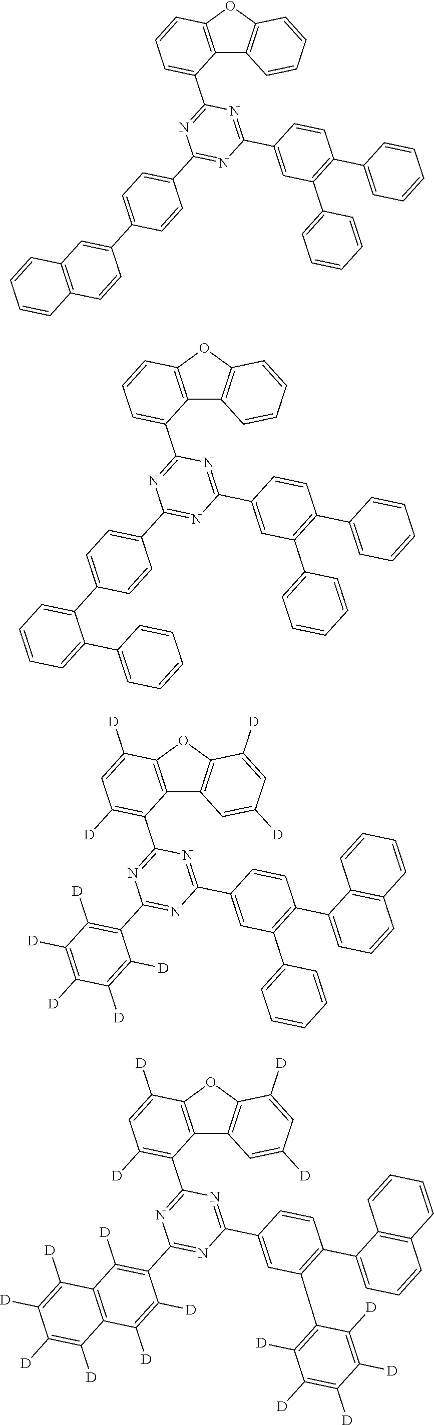

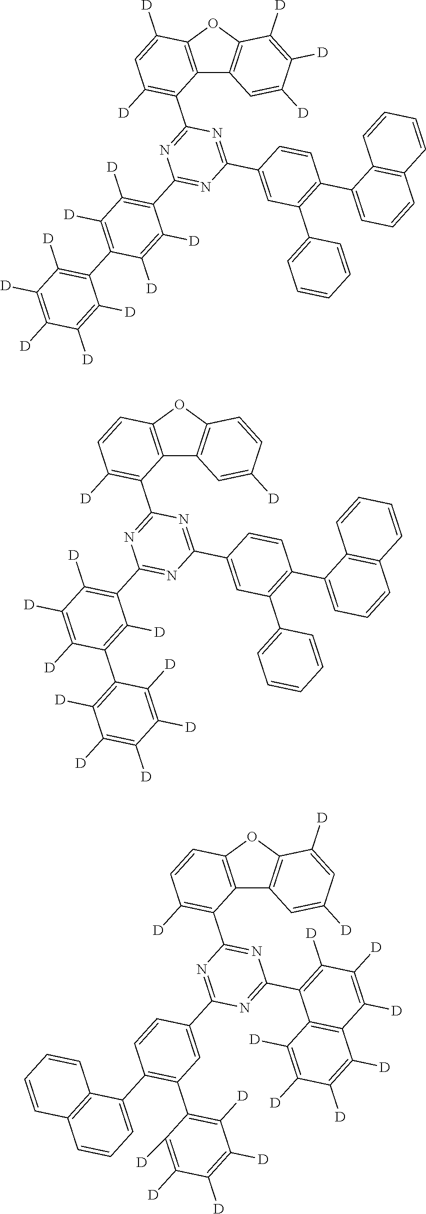 [SMILES: [2H]c1cc2c(oc3c([2H])cc([2H])c(-c4nc(-c5ccc(-c6cccc7ccccc67)c(-c6ccccc6)c5)nc(-c5c([2H])c([2H])c(-c6c([2H])c([2H])c([2H])c([2H])c6[2H])c([2H])c5[2H])n4)c32)c([2H])c1[2H] ; [2H]c1ccc2oc3ccc([2H])c(-c4nc(-c5ccc(-c6cccc7ccccc67)c(-c6ccccc6)c5)nc(-c5c([2H])c([2H])c([2H])c(-c6c([2H])c([2H])c([2H])c([2H])c6[2H])c5[2H])n4)c3c2c1 ; [2H]c1cc([2H])c2oc3ccc([2H])c(-c4nc(-c5ccc(-c6cccc7ccccc67)c(-c6c([2H])c([2H])c([2H])c([2H])c6[2H])c5)nc(-c5c([2H])c([2H])c([2H])c6c([2H])c([2H])c([2H])c([2H])c56)n4)c3c2c1]

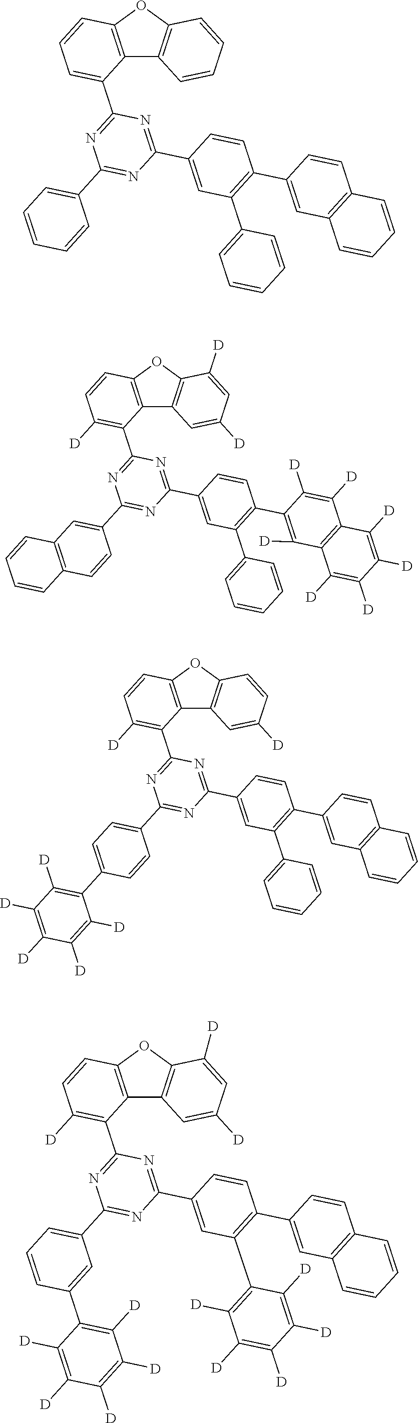

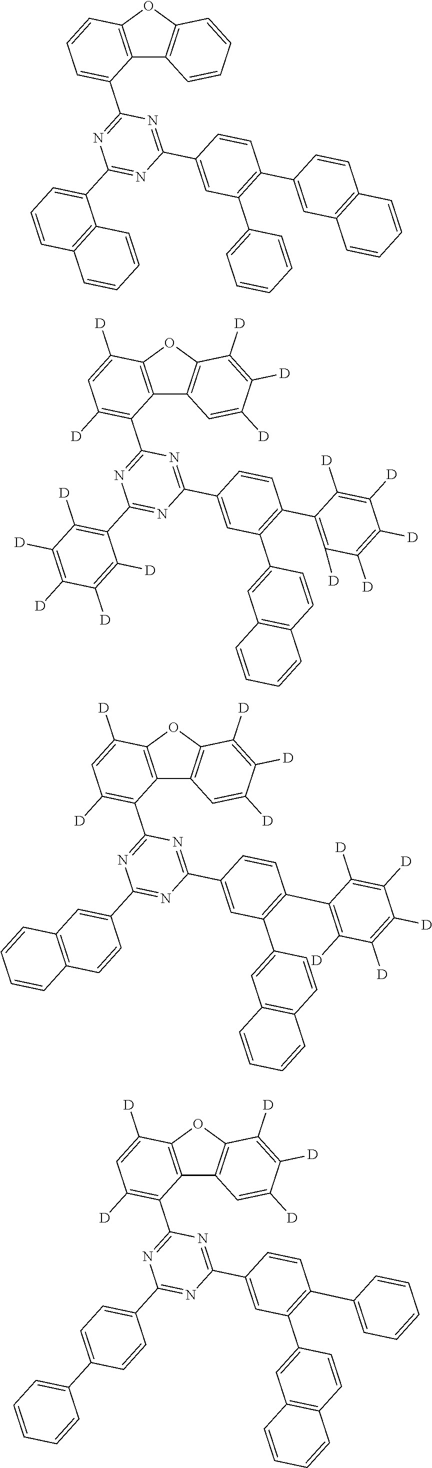

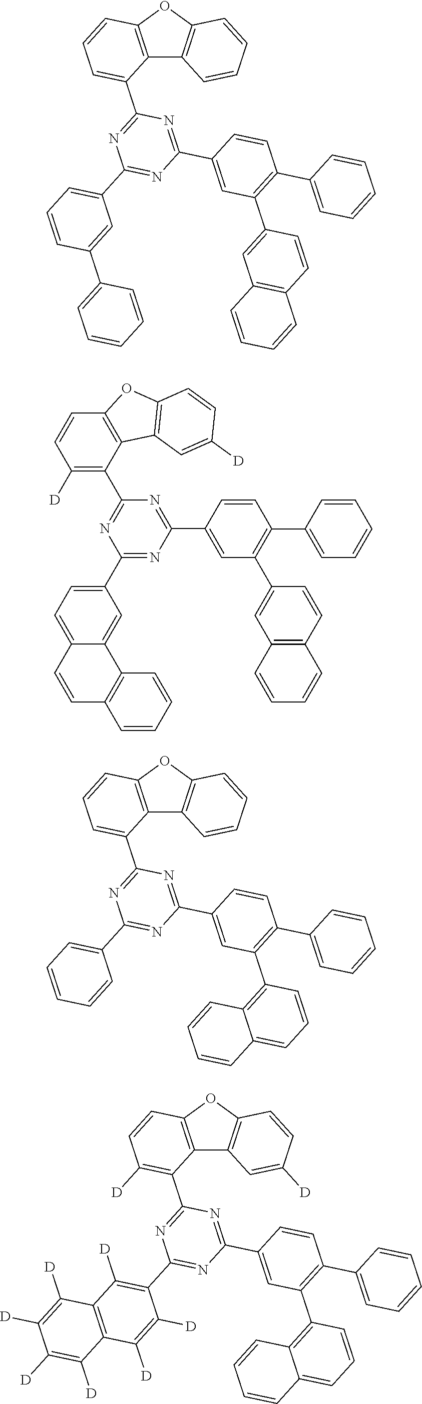

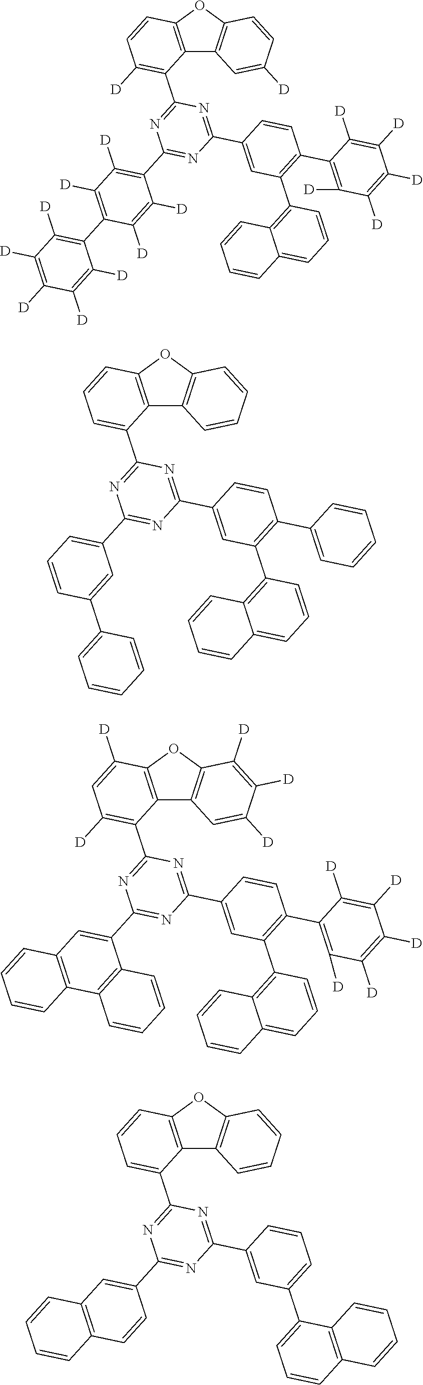

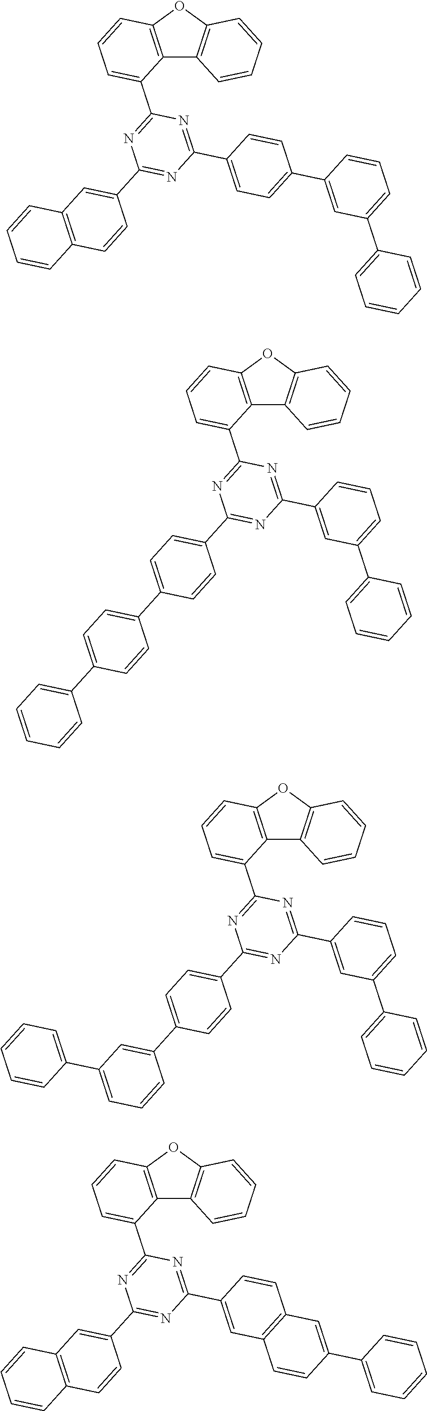 [SMILES: c1ccc(-c2cccc(-c3ccc(-c4nc(-c5ccc6ccccc6c5)nc(-c5cccc6oc7ccccc7c56)n4)cc3)c2)cc1 ; c1ccc(-c2ccc(-c3ccc(-c4nc(-c5cccc(-c6ccccc6)c5)nc(-c5cccc6oc7ccccc7c56)n4)cc3)cc2)cc1 ; c1ccc(-c2cccc(-c3ccc(-c4nc(-c5cccc(-c6ccccc6)c5)nc(-c5cccc6oc7ccccc7c56)n4)cc3)c2)cc1 ; c1ccc(-c2ccc3cc(-c4nc(-c5ccc6ccccc6c5)nc(-c5cccc6oc7ccccc7c56)n4)ccc3c2)cc1]

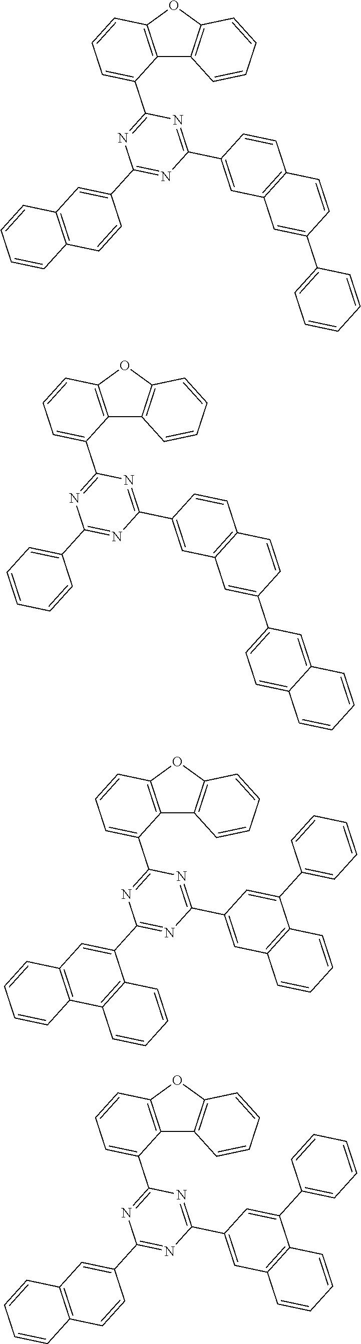 [SMILES: c1ccc(-c2ccc3ccc(-c4nc(-c5ccc6ccccc6c5)nc(-c5cccc6oc7ccccc7c56)n4)cc3c2)cc1 ; c1ccc(-c2nc(-c3ccc4ccc(-c5ccc6ccccc6c5)cc4c3)nc(-c3cccc4oc5ccccc5c34)n2)cc1 ; c1ccc(-c2cc(-c3nc(-c4cc5ccccc5c5ccccc45)nc(-c4cccc5oc6ccccc6c45)n3)cc3ccccc23)cc1 ; c1ccc(-c2cc(-c3nc(-c4ccc5ccccc5c4)nc(-c4cccc5oc6ccccc6c45)n3)cc3ccccc23)cc1]

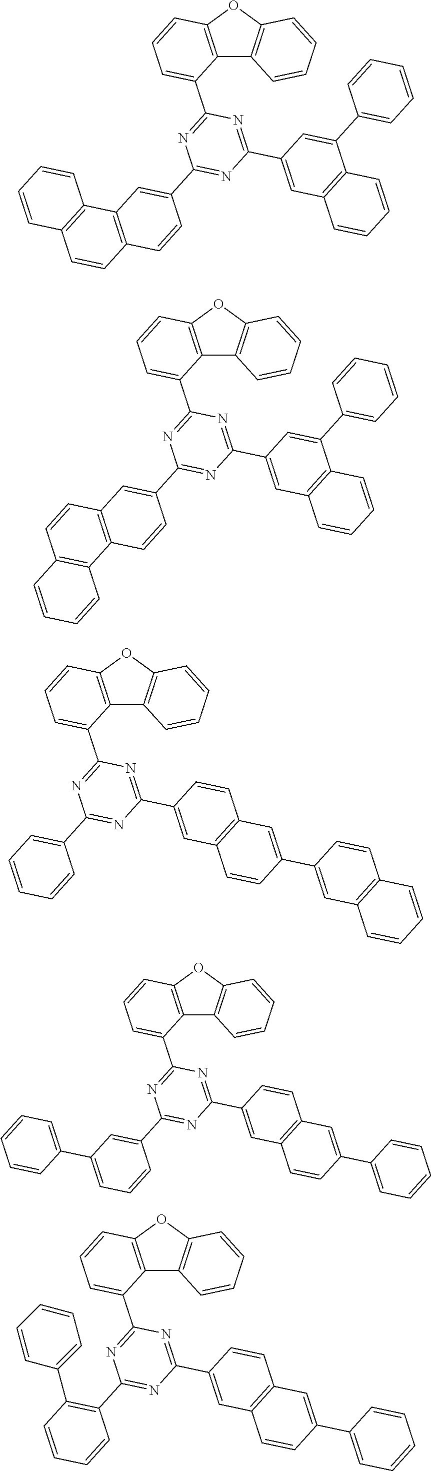

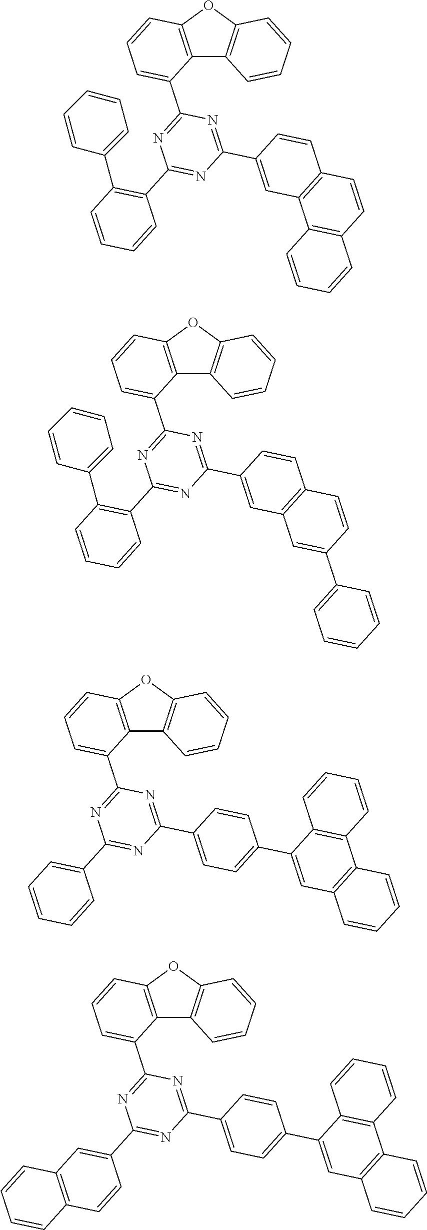 [SMILES: c1ccc(-c2ccccc2-c2nc(-c3ccc4ccc5ccccc5c4c3)nc(-c3cccc4oc5ccccc5c34)n2)cc1 ; c1ccc(-c2ccc3ccc(-c4nc(-c5ccccc5-c5ccccc5)nc(-c5cccc6oc7ccccc7c56)n4)cc3c2)cc1 ; c1ccc(-c2nc(-c3ccc(-c4cc5ccccc5c5ccccc45)cc3)nc(-c3cccc4oc5ccccc5c34)n2)cc1 ; c1ccc2cc(-c3nc(-c4ccc(-c5cc6ccccc6c6ccccc56)cc4)nc(-c4cccc5oc6ccccc6c45)n3)ccc2c1]

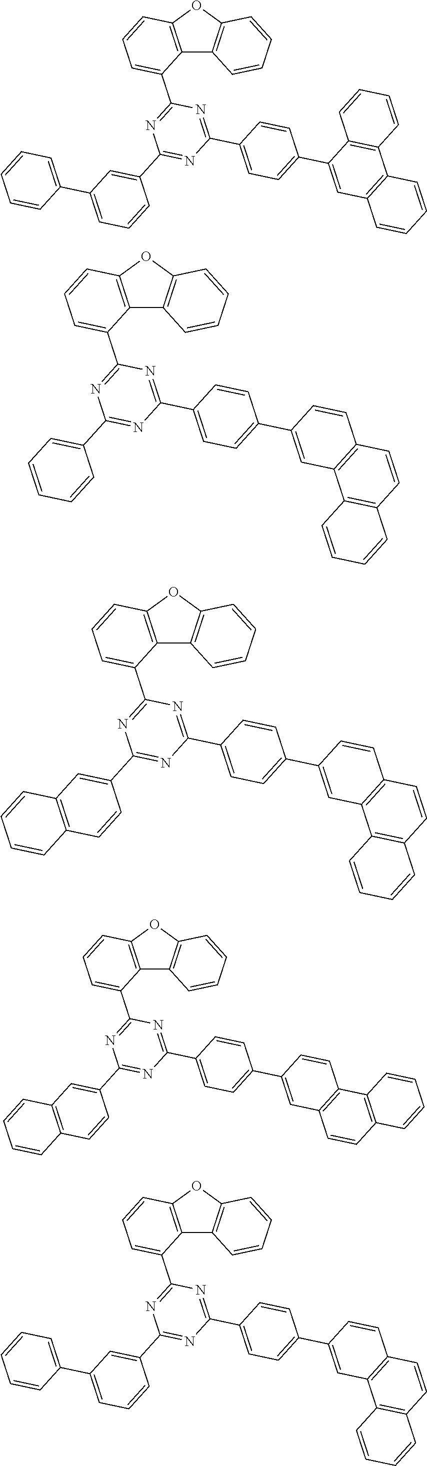

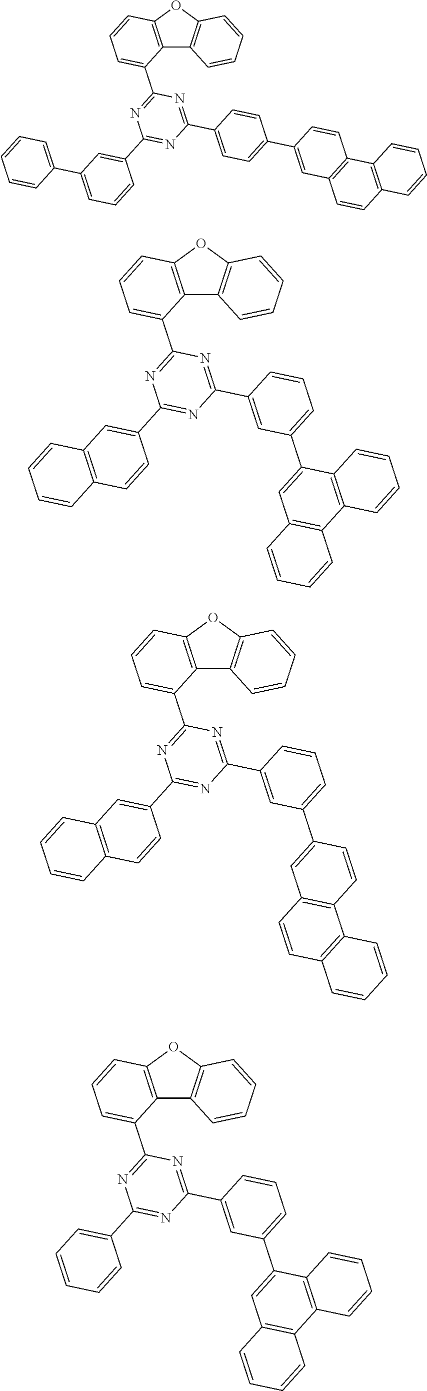 [SMILES: c1ccc(-c2cccc(-c3nc(-c4ccc(-c5ccc6c(ccc7ccccc76)c5)cc4)nc(-c4cccc5oc6ccccc6c45)n3)c2)cc1 ; c1cc(-c2nc(-c3ccc4ccccc4c3)nc(-c3cccc4oc5ccccc5c34)n2)cc(-c2cc3ccccc3c3ccccc23)c1 ; c1cc(-c2ccc3c(ccc4ccccc43)c2)cc(-c2nc(-c3ccc4ccccc4c3)nc(-c3cccc4oc5ccccc5c34)n2)c1 ; c1ccc(-c2nc(-c3cccc(-c4cc5ccccc5c5ccccc45)c3)nc(-c3cccc4oc5ccccc5c34)n2)cc1]

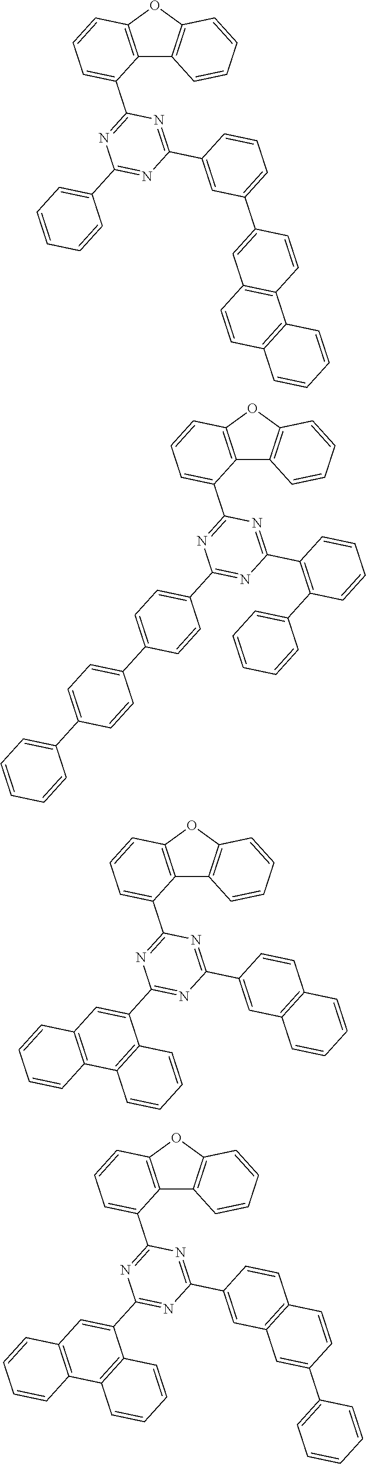

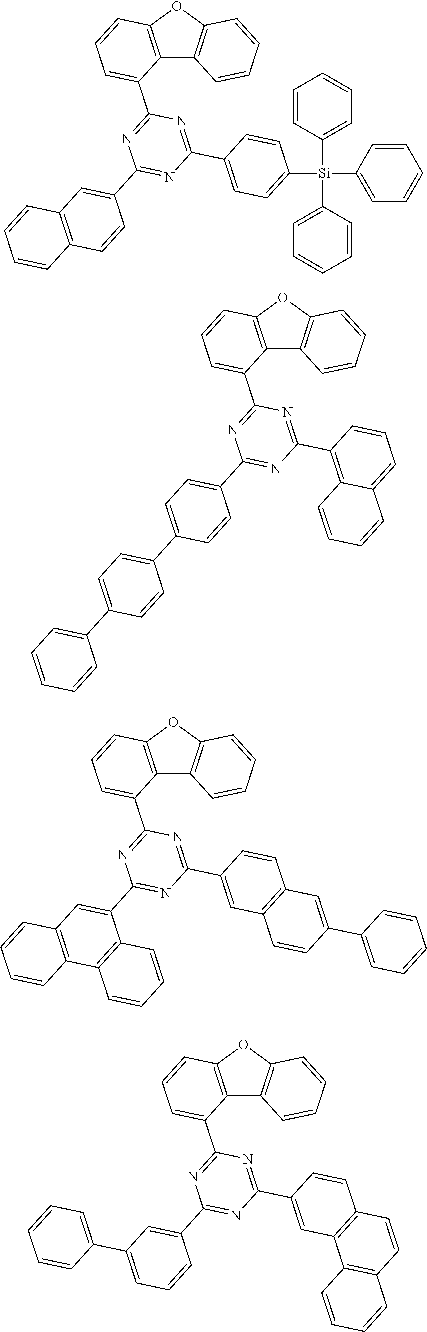

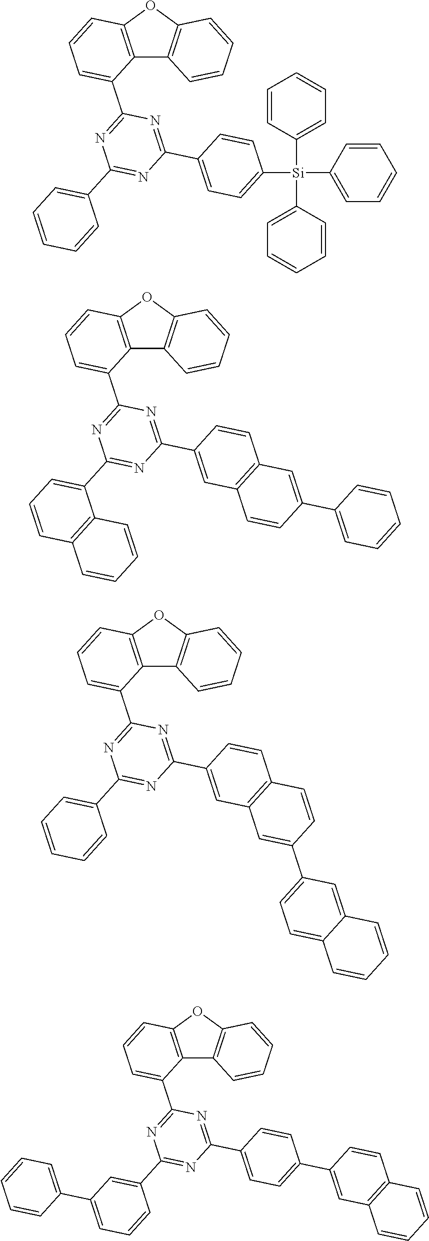 [SMILES: c1ccc(-c2nc(-c3ccc([Si](c4ccccc4)(c4ccccc4)c4ccccc4)cc3)nc(-c3cccc4oc5ccccc5c34)n2)cc1 ; c1ccc(-c2ccc3cc(-c4nc(-c5cccc6ccccc56)nc(-c5cccc6oc7ccccc7c56)n4)ccc3c2)cc1 ; c1ccc(-c2nc(-c3ccc4ccc(-c5ccc6ccccc6c5)cc4c3)nc(-c3cccc4oc5ccccc5c34)n2)cc1 ; c1ccc(-c2cccc(-c3nc(-c4ccc(-c5ccc6ccccc6c5)cc4)nc(-c4cccc5oc6ccccc6c45)n3)c2)cc1]

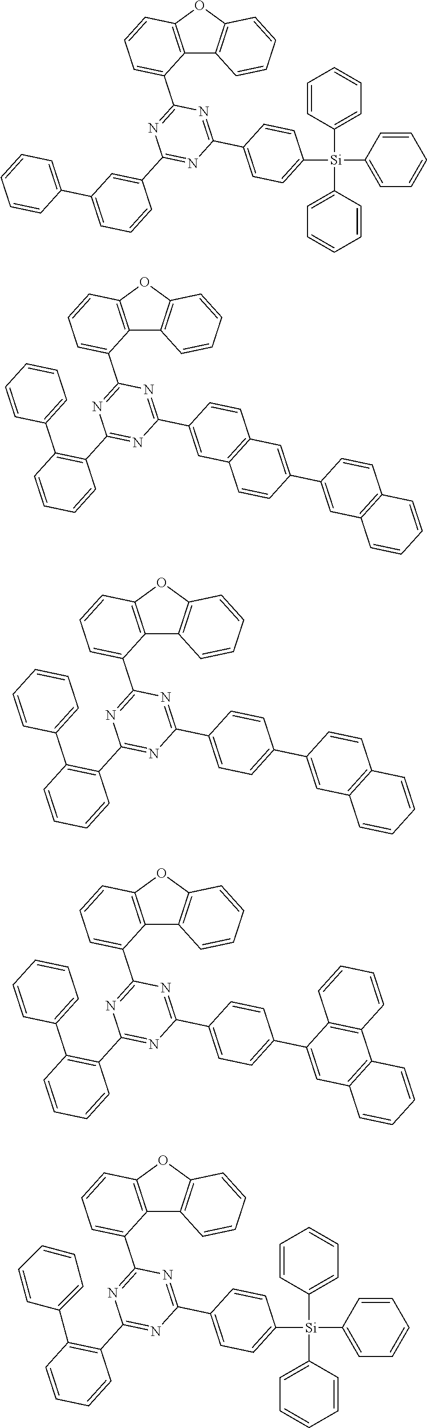 [SMILES: c1ccc(-c2cccc(-c3nc(-c4ccc([Si](c5ccccc5)(c5ccccc5)c5ccccc5)cc4)nc(-c4cccc5oc6ccccc6c45)n3)c2)cc1 ; c1ccc(-c2ccccc2-c2nc(-c3ccc4cc(-c5ccc6ccccc6c5)ccc4c3)nc(-c3cccc4oc5ccccc5c34)n2)cc1 ; c1ccc(-c2ccccc2-c2nc(-c3ccc(-c4ccc5ccccc5c4)cc3)nc(-c3cccc4oc5ccccc5c34)n2)cc1 ; c1ccc(-c2ccccc2-c2nc(-c3ccc(-c4cc5ccccc5c5ccccc45)cc3)nc(-c3cccc4oc5ccccc5c34)n2)cc1 ; c1ccc(-c2ccccc2-c2nc(-c3ccc([Si](c4ccccc4)(c4ccccc4)c4ccccc4)cc3)nc(-c3cccc4oc5ccccc5c34)n2)cc1]

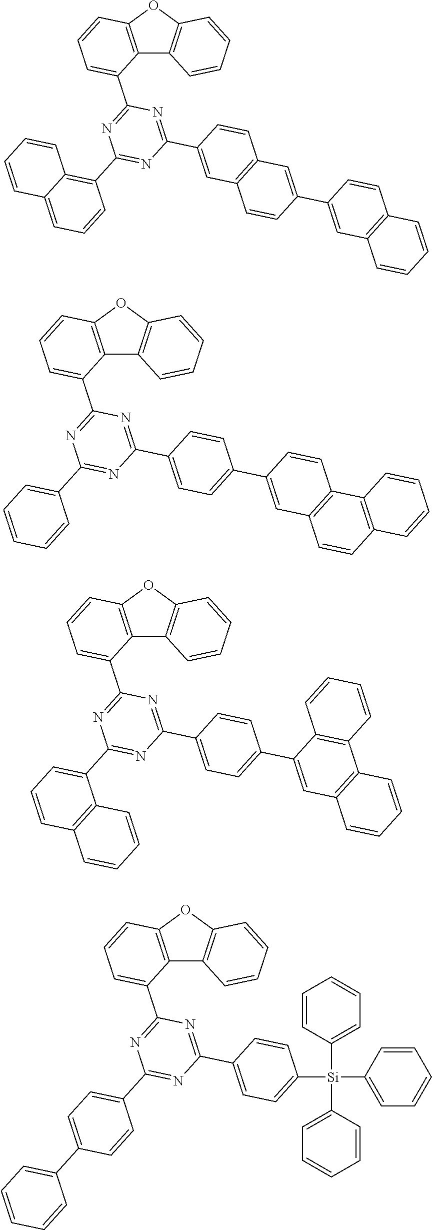 [SMILES: c1ccc2cc(-c3ccc4cc(-c5nc(-c6cccc7ccccc67)nc(-c6cccc7oc8ccccc8c67)n5)ccc4c3)ccc2c1 ; c1ccc(-c2nc(-c3ccc(-c4ccc5c(ccc6ccccc65)c4)cc3)nc(-c3cccc4oc5ccccc5c34)n2)cc1 ; c1ccc2c(-c3nc(-c4ccc(-c5cc6ccccc6c6ccccc56)cc4)nc(-c4cccc5oc6ccccc6c45)n3)cccc2c1 ; c1ccc(-c2ccc(-c3nc(-c4ccc([Si](c5ccccc5)(c5ccccc5)c5ccccc5)cc4)nc(-c4cccc5oc6ccccc6c45)n3)cc2)cc1]

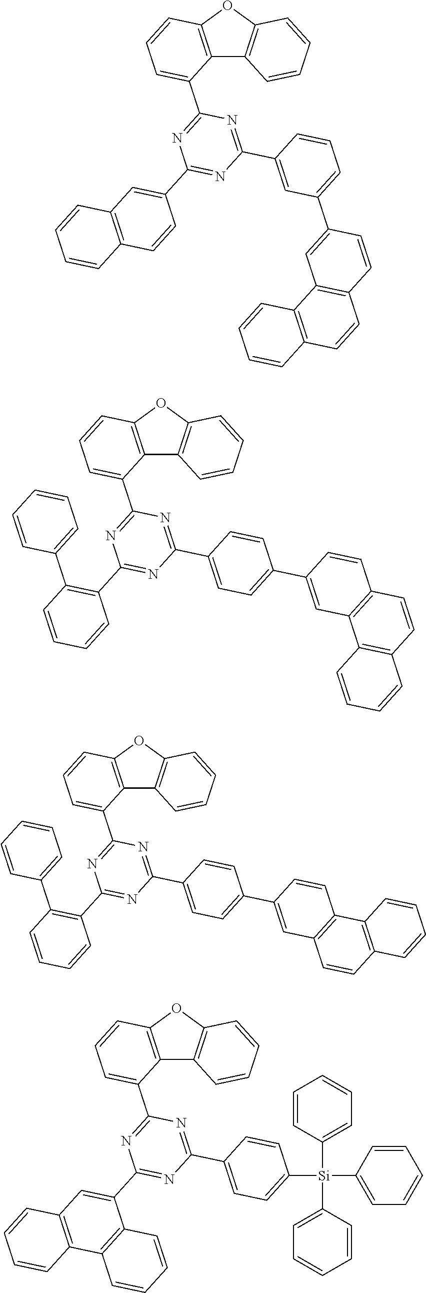 [SMILES: c1cc(-c2ccc3ccc4ccccc4c3c2)cc(-c2nc(-c3ccc4ccccc4c3)nc(-c3cccc4oc5ccccc5c34)n2)c1 ; c1ccc(-c2ccccc2-c2nc(-c3ccc(-c4ccc5ccc6ccccc6c5c4)cc3)nc(-c3cccc4oc5ccccc5c34)n2)cc1 ; c1ccc(-c2ccccc2-c2nc(-c3ccc(-c4ccc5c(ccc6ccccc65)c4)cc3)nc(-c3cccc4oc5ccccc5c34)n2)cc1 ; c1ccc([Si](c2ccccc2)(c2ccccc2)c2ccc(-c3nc(-c4cc5ccccc5c5ccccc45)nc(-c4cccc5oc6ccccc6c45)n3)cc2)cc1]

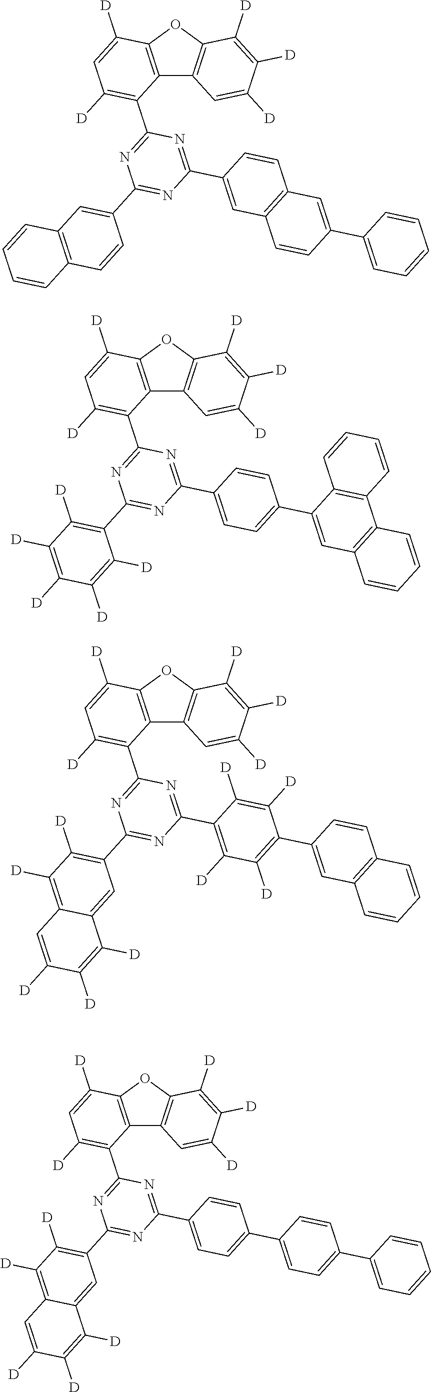

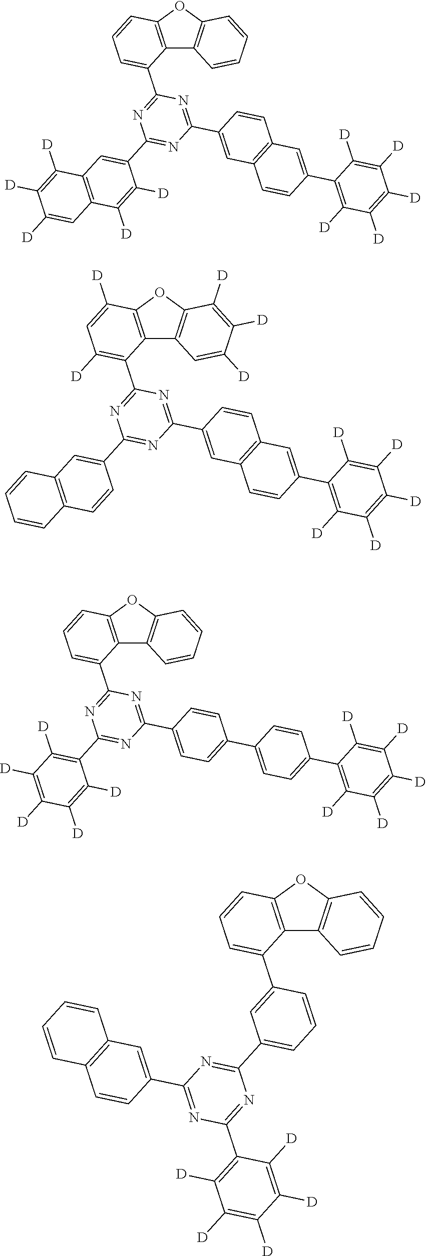 [SMILES: [2H]c1cc2c([2H])c([2H])c(-c3nc(-c4ccc5cc(-c6c([2H])c([2H])c([2H])c([2H])c6[2H])ccc5c4)nc(-c4cccc5oc6ccccc6c45)n3)cc2c([2H])c1[2H] ; [2H]c1cc2c(oc3c([2H])cc([2H])c(-c4nc(-c5ccc6ccccc6c5)nc(-c5ccc6cc(-c7c([2H])c([2H])c([2H])c([2H])c7[2H])ccc6c5)n4)c32)c([2H])c1[2H] ; [2H]c1c([2H])c([2H])c(-c2ccc(-c3ccc(-c4nc(-c5c([2H])c([2H])c([2H])c([2H])c5[2H])nc(-c5cccc6oc7ccccc7c56)n4)cc3)cc2)c([2H])c1[2H] ; [2H]c1c([2H])c([2H])c(-c2nc(-c3cccc(-c4cccc5oc6ccccc6c45)c3)nc(-c3ccc4ccccc4c3)n2)c([2H])c1[2H]]

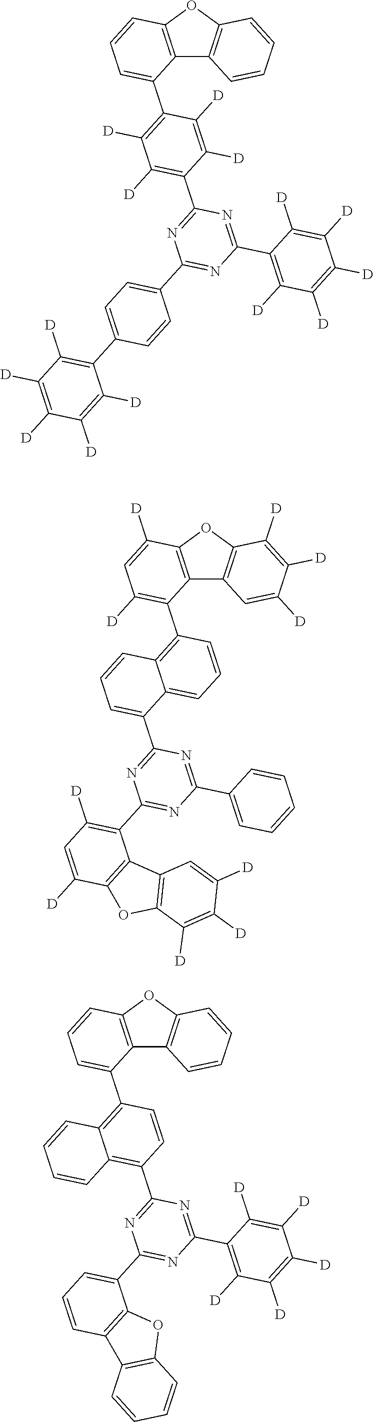

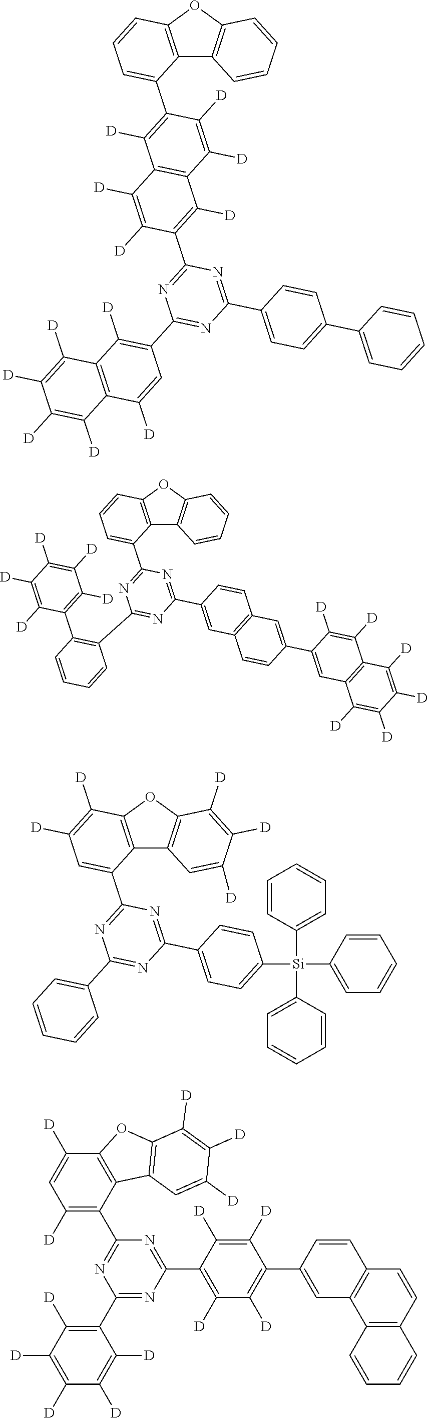

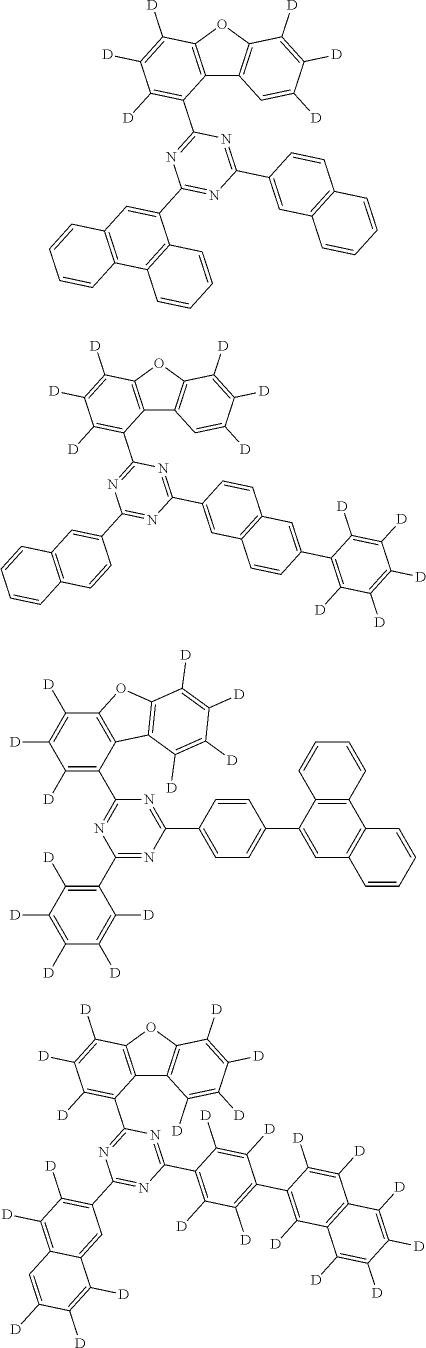

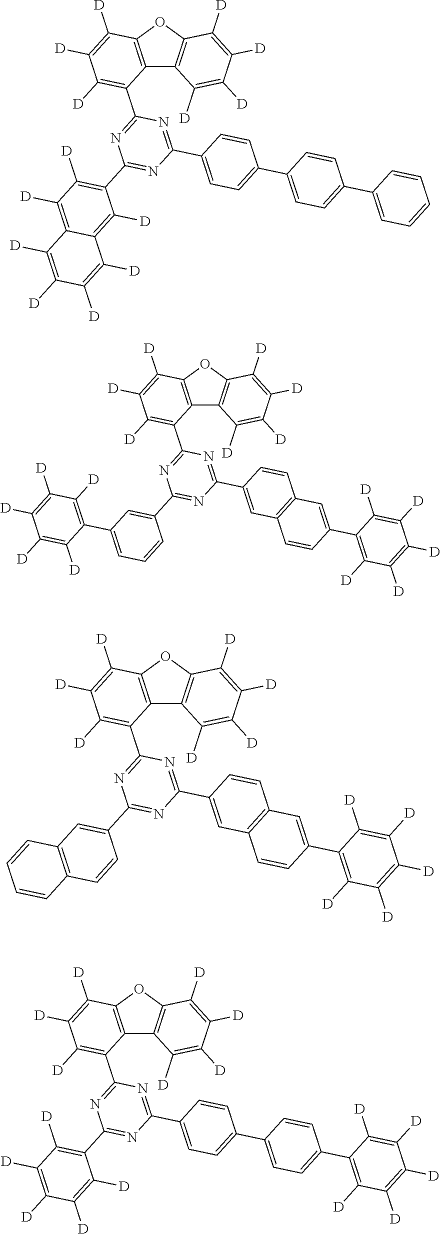

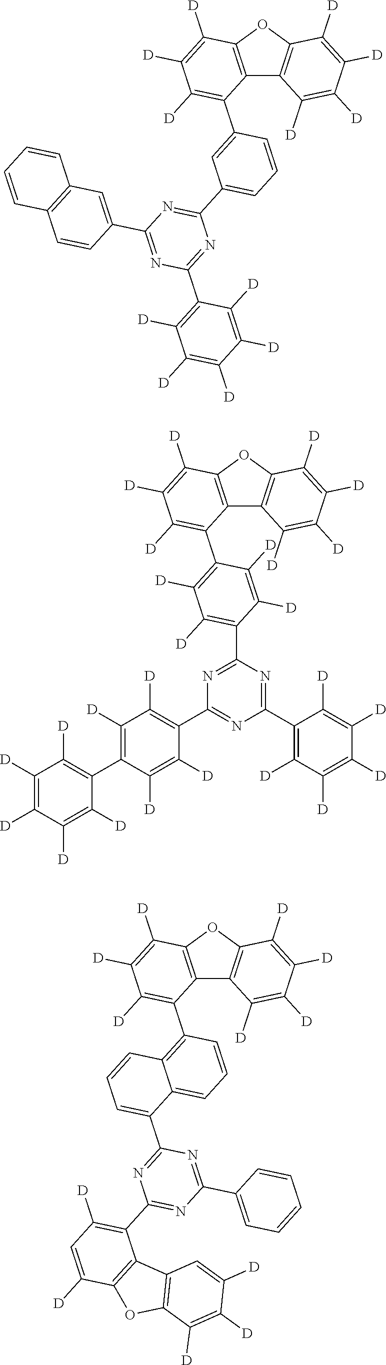

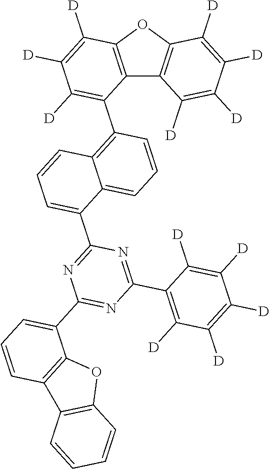 [SMILES: [2H]c1c([2H])c([2H])c(-c2nc(-c3cccc4c(-c5c([2H])c([2H])c([2H])c6oc7c([2H])c([2H])c([2H])c([2H])c7c56)cccc34)nc(-c3cccc4c3oc3ccccc34)n2)c([2H])c1[2H]]

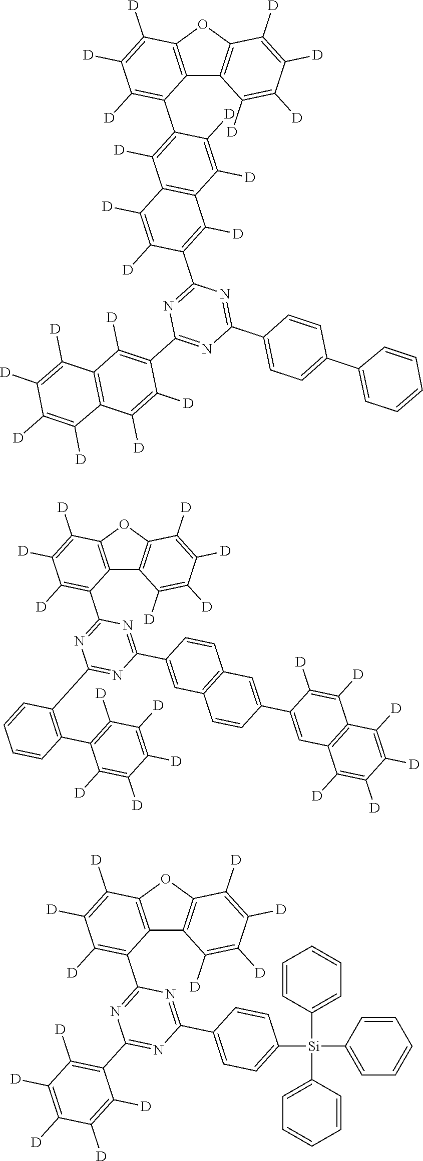

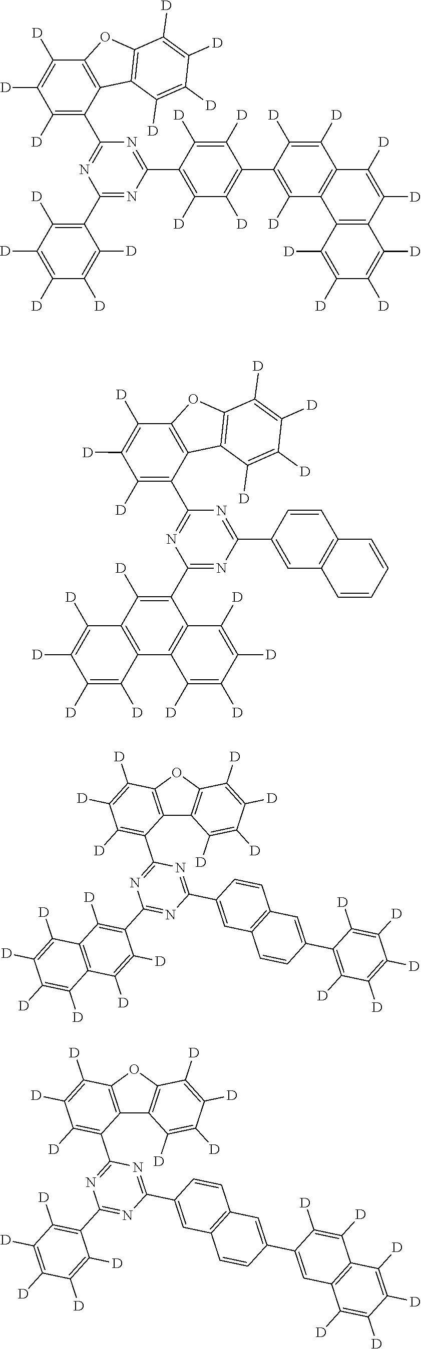

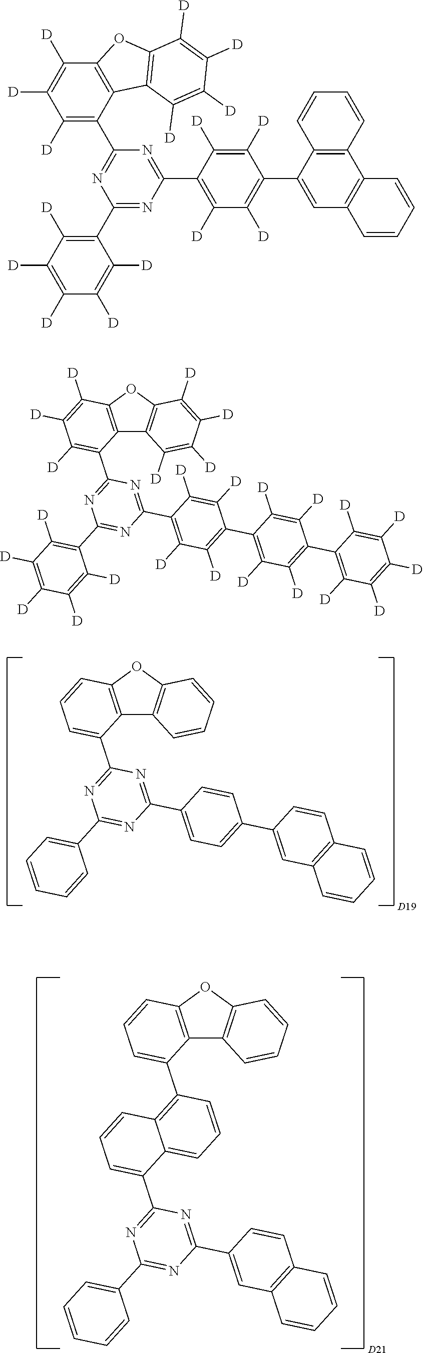

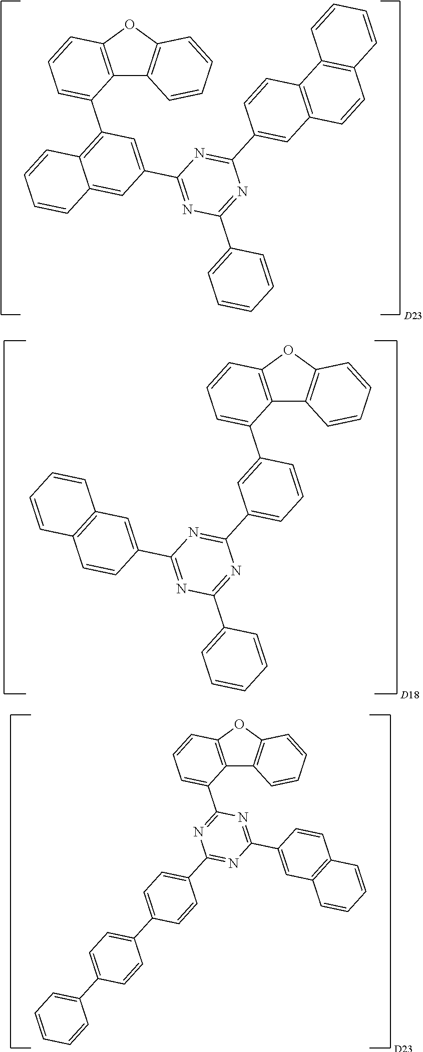

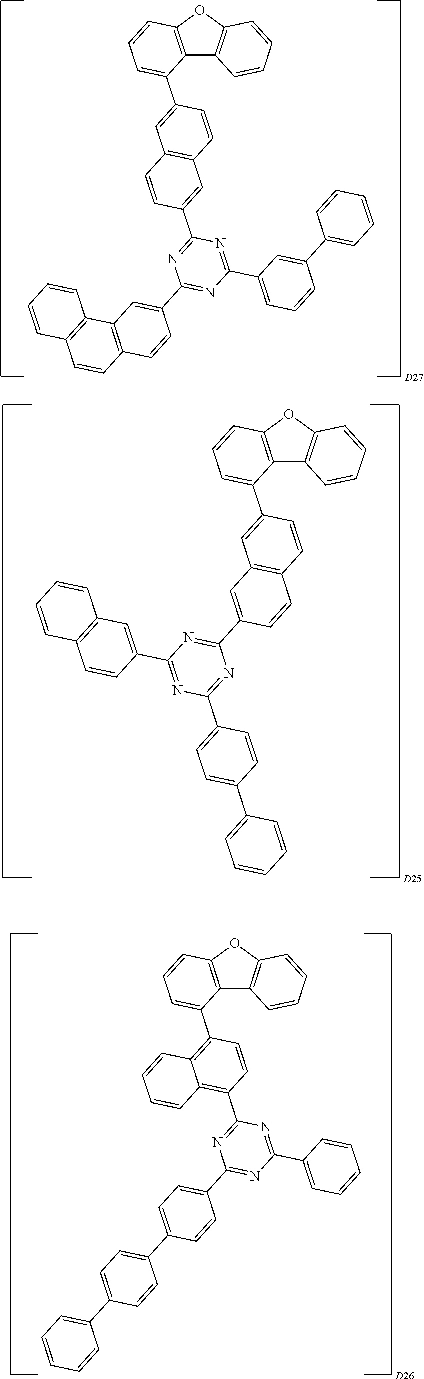

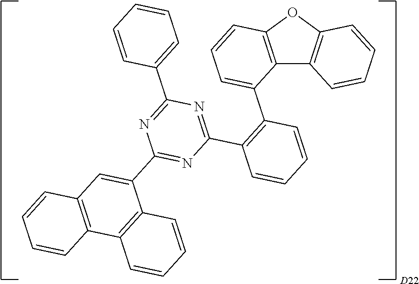

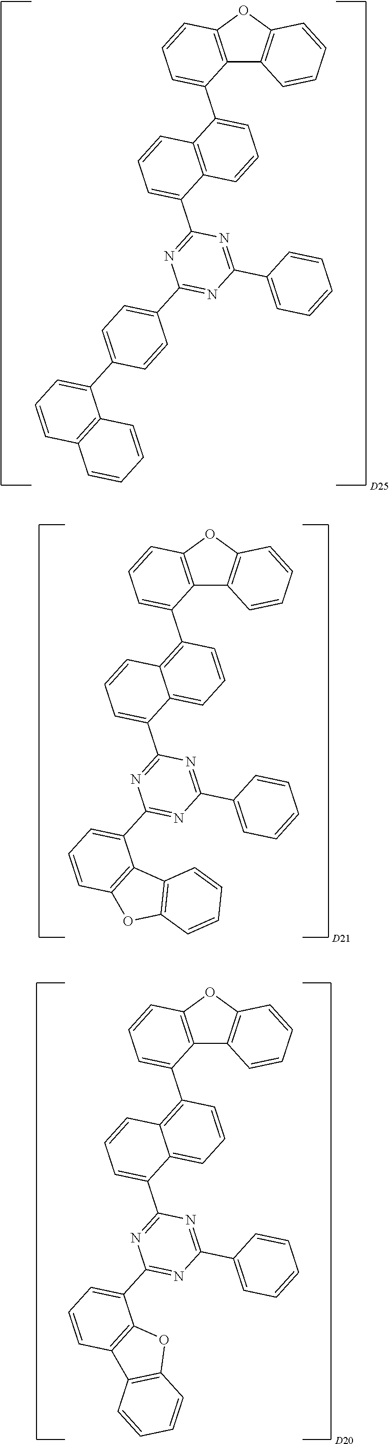

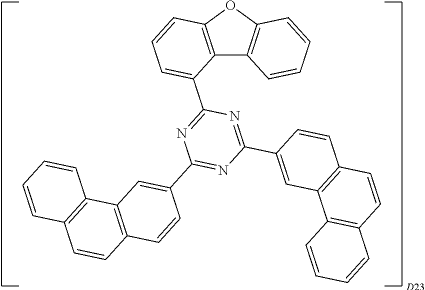

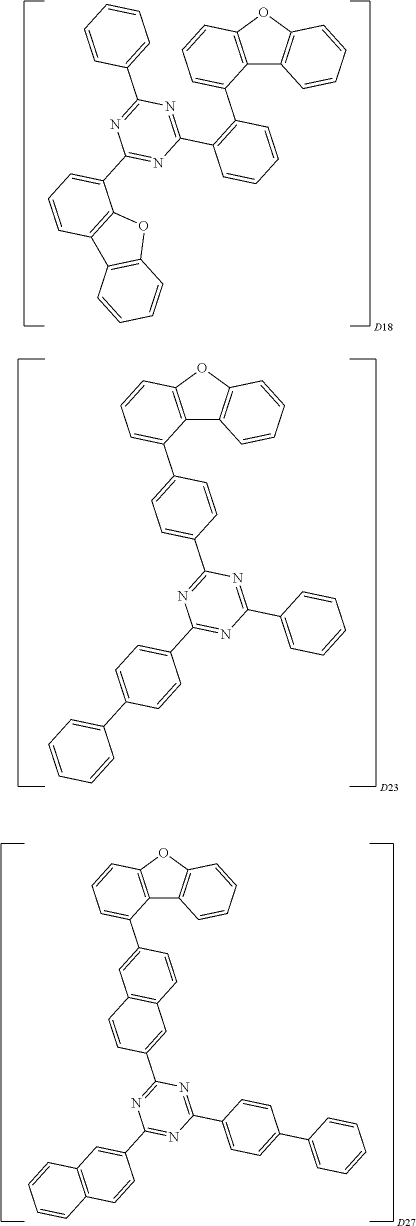

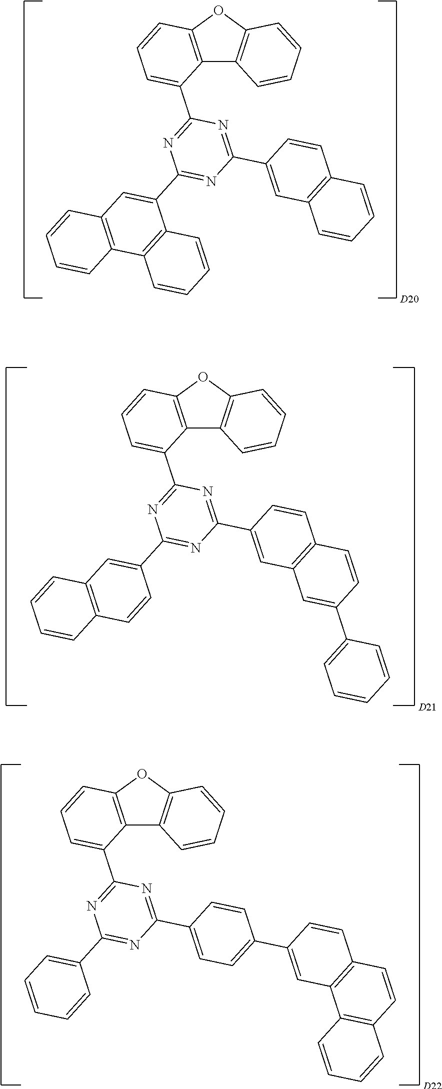

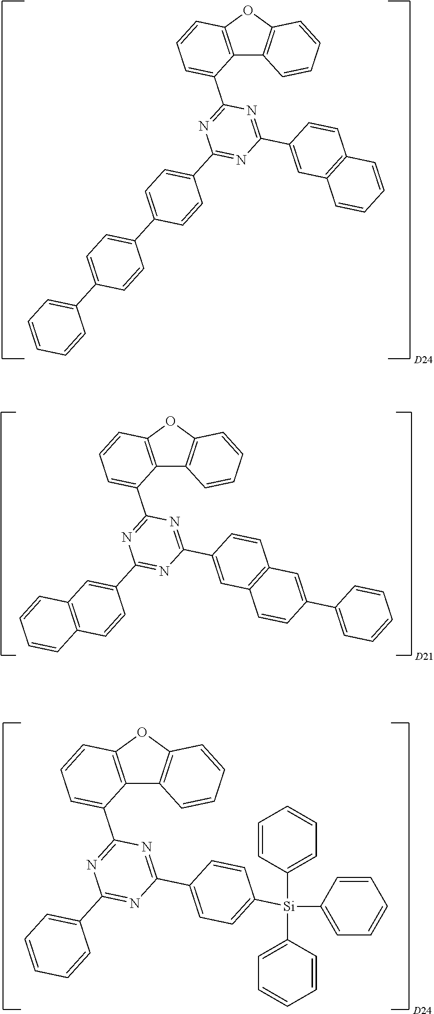

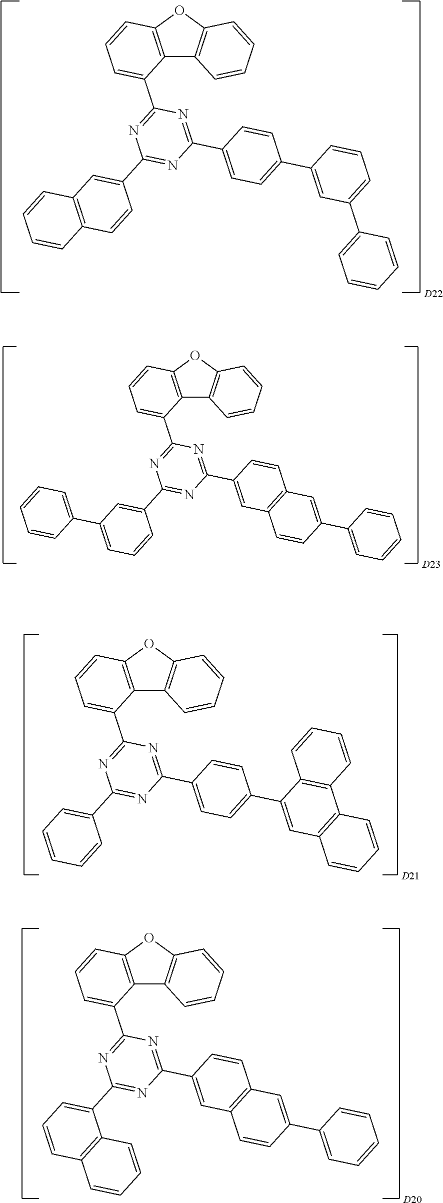

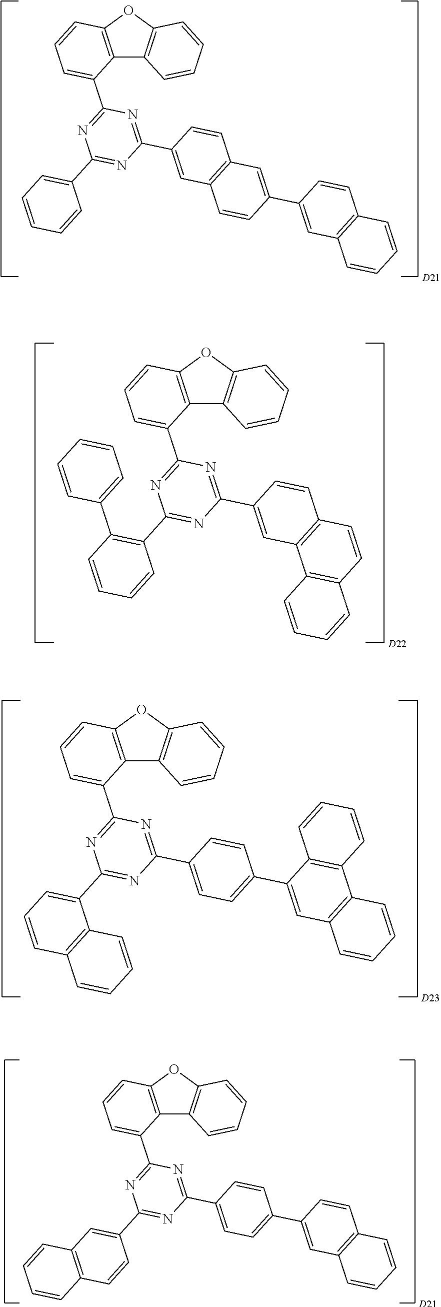

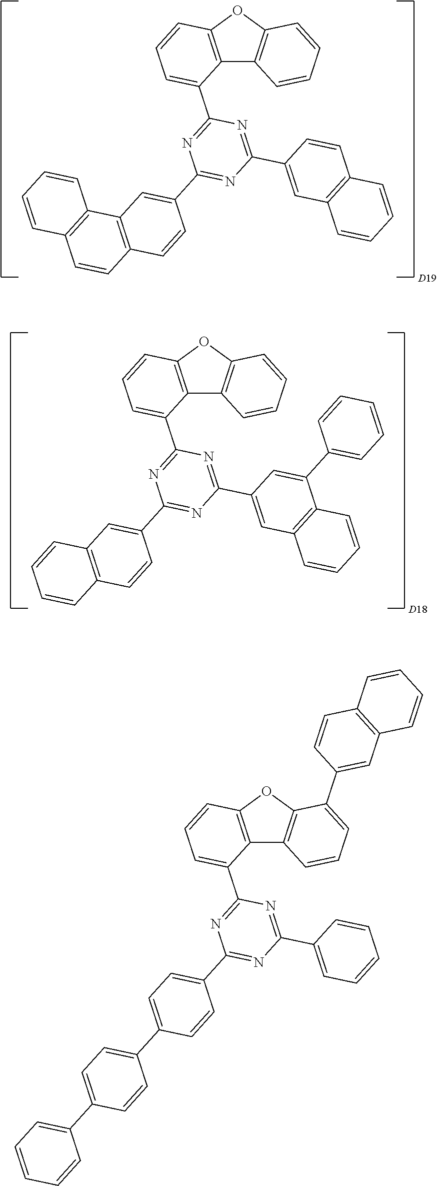

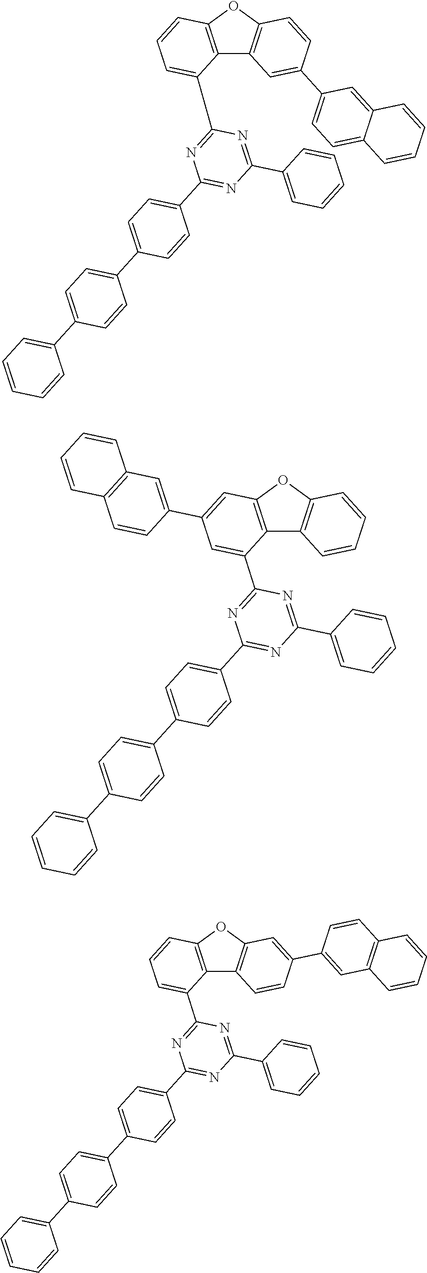 [SMILES: c1ccc(-c2ccc(-c3ccc(-c4nc(-c5ccccc5)nc(-c5cccc6oc7ccc(-c8ccc9ccccc9c8)cc7c56)n4)cc3)cc2)cc1 ; c1ccc(-c2ccc(-c3ccc(-c4nc(-c5ccccc5)nc(-c5cc(-c6ccc7ccccc7c6)cc6oc7ccccc7c56)n4)cc3)cc2)cc1 ; c1ccc(-c2ccc(-c3ccc(-c4nc(-c5ccccc5)nc(-c5cccc6oc7cc(-c8ccc9ccccc9c8)ccc7c56)n4)cc3)cc2)cc1]

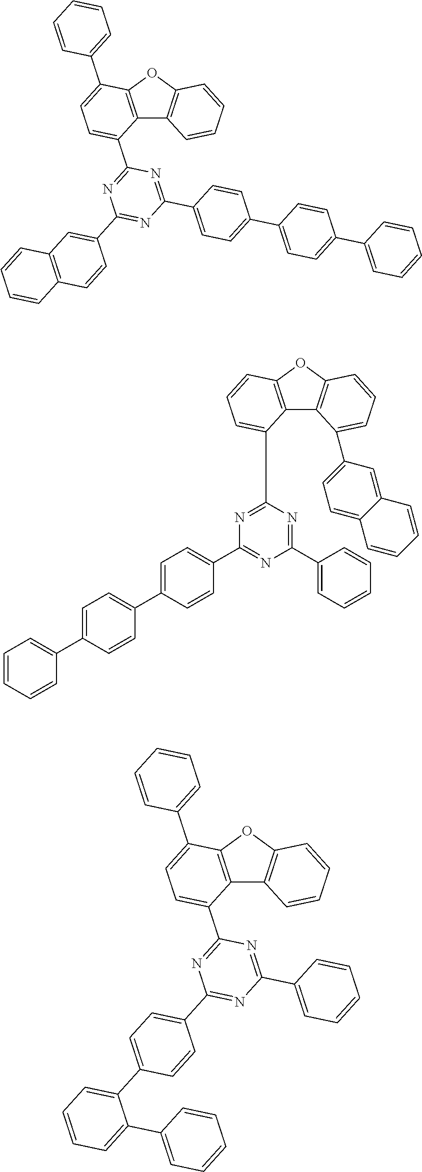 [SMILES: c1ccc(-c2ccc(-c3ccc(-c4nc(-c5ccc6ccccc6c5)nc(-c5ccc(-c6ccccc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9ccccc9c8)c7c56)n4)cc3)cc2)cc1 ; c1ccc(-c2nc(-c3ccc(-c4ccccc4-c4ccccc4)cc3)nc(-c3ccc(-c4ccccc4)c4oc5ccccc5c34)n2)cc1]

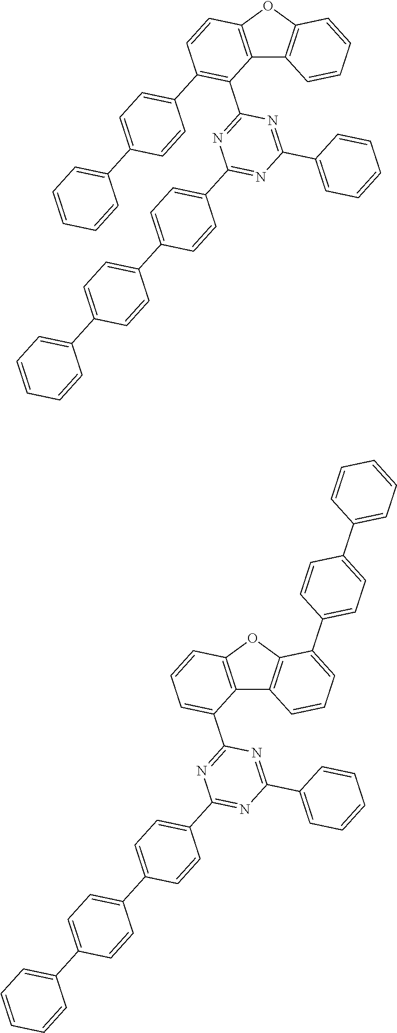 [SMILES: c1ccc(-c2ccc(-c3ccc(-c4nc(-c5ccccc5)nc(-c5c(-c6ccc(-c7ccccc7)cc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccc(-c9ccccc9)cc8)cccc7c56)n4)cc3)cc2)cc1]

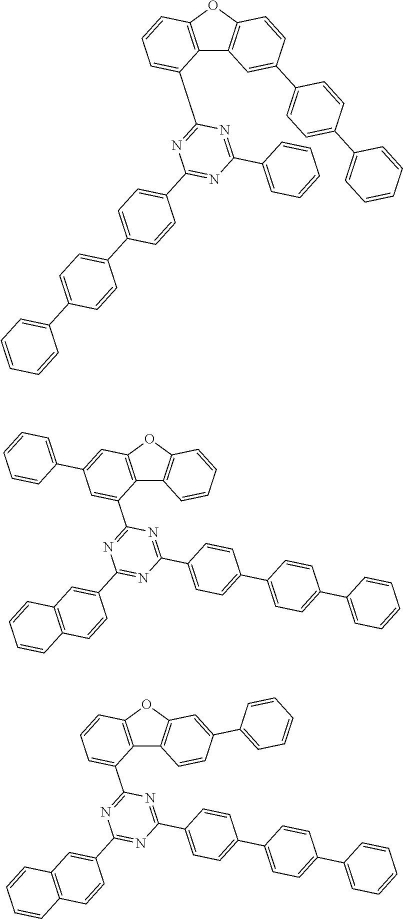 [SMILES: c1ccc(-c2ccc(-c3ccc(-c4nc(-c5ccccc5)nc(-c5cccc6oc7ccc(-c8ccc(-c9ccccc9)cc8)cc7c56)n4)cc3)cc2)cc1 ; c1ccc(-c2ccc(-c3ccc(-c4nc(-c5ccc6ccccc6c5)nc(-c5cc(-c6ccccc6)cc6oc7ccccc7c56)n4)cc3)cc2)cc1 ; c1ccc(-c2ccc(-c3ccc(-c4nc(-c5ccc6ccccc6c5)nc(-c5cccc6oc7cc(-c8ccccc8)ccc7c56)n4)cc3)cc2)cc1]

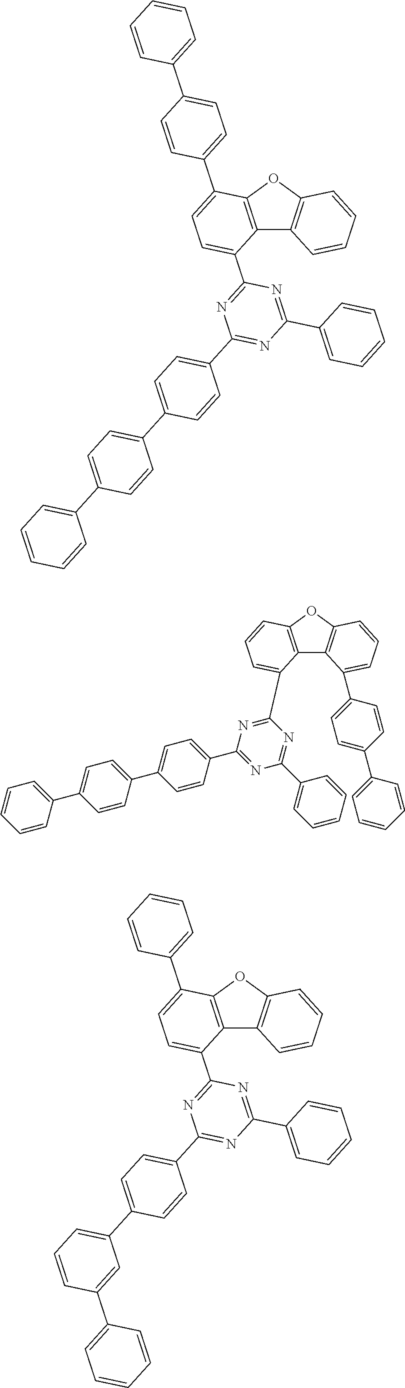 [SMILES: c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1 ; c1ccc(-c2cccc(-c3ccc(-c4nc(-c5ccccc5)nc(-c5ccc(-c6ccccc6)c6oc7ccccc7c56)n4)cc3)c2)cc1]

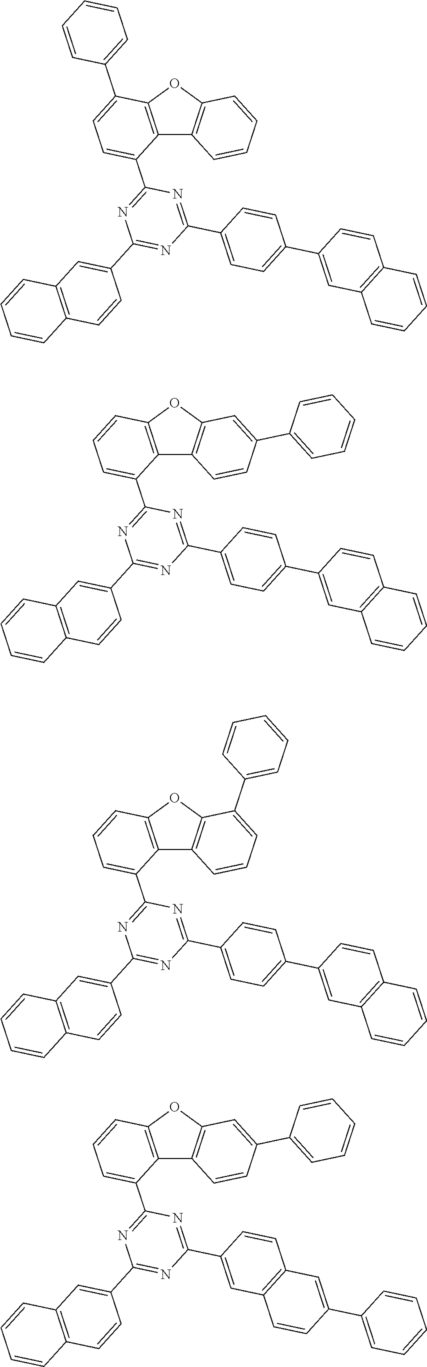 [SMILES: c1ccc(-c2ccc(-c3nc(-c4ccc(-c5ccc6ccccc6c5)cc4)nc(-c4ccc5ccccc5c4)n3)c3c2oc2ccccc23)cc1 ; c1ccc(-c2ccc3c(c2)oc2cccc(-c4nc(-c5ccc(-c6ccc7ccccc7c6)cc5)nc(-c5ccc6ccccc6c5)n4)c23)cc1 ; c1ccc(-c2cccc3c2oc2cccc(-c4nc(-c5ccc(-c6ccc7ccccc7c6)cc5)nc(-c5ccc6ccccc6c5)n4)c23)cc1 ; c1ccc(-c2ccc3cc(-c4nc(-c5ccc6ccccc6c5)nc(-c5cccc6oc7cc(-c8ccccc8)ccc7c56)n4)ccc3c2)cc1]

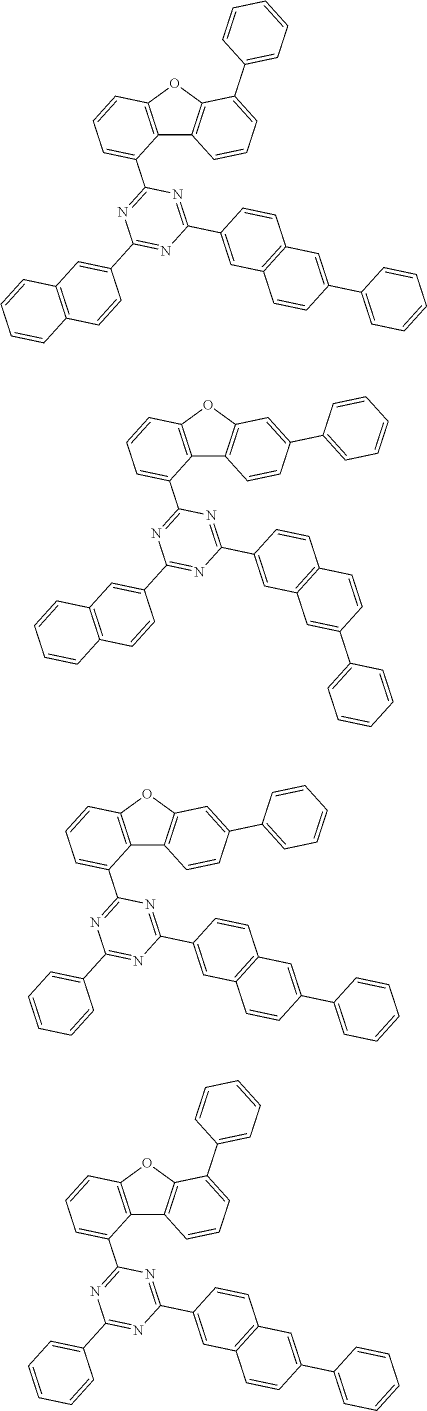 [SMILES: c1ccc(-c2ccc3cc(-c4nc(-c5ccc6ccccc6c5)nc(-c5cccc6oc7c(-c8ccccc8)cccc7c56)n4)ccc3c2)cc1 ; c1ccc(-c2ccc3ccc(-c4nc(-c5ccc6ccccc6c5)nc(-c5cccc6oc7cc(-c8ccccc8)ccc7c56)n4)cc3c2)cc1 ; c1ccc(-c2ccc3cc(-c4nc(-c5ccccc5)nc(-c5cccc6oc7cc(-c8ccccc8)ccc7c56)n4)ccc3c2)cc1 ; c1ccc(-c2ccc3cc(-c4nc(-c5ccccc5)nc(-c5cccc6oc7c(-c8ccccc8)cccc7c56)n4)ccc3c2)cc1]

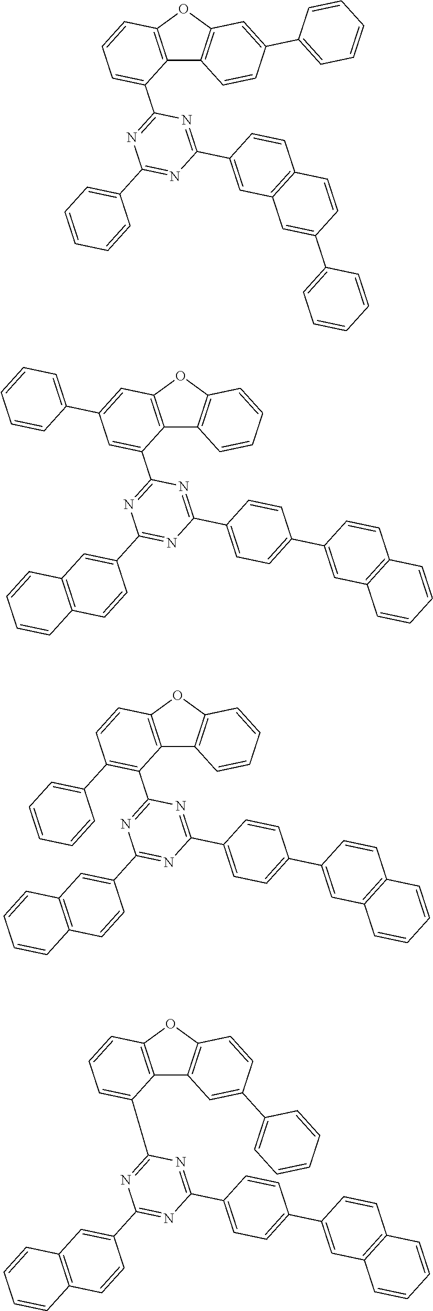 [SMILES: c1ccc(-c2ccc3ccc(-c4nc(-c5ccccc5)nc(-c5cccc6oc7cc(-c8ccccc8)ccc7c56)n4)cc3c2)cc1 ; c1ccc(-c2cc(-c3nc(-c4ccc(-c5ccc6ccccc6c5)cc4)nc(-c4ccc5ccccc5c4)n3)c3c(c2)oc2ccccc23)cc1 ; c1ccc(-c2ccc3oc4ccccc4c3c2-c2nc(-c3ccc(-c4ccc5ccccc5c4)cc3)nc(-c3ccc4ccccc4c3)n2)cc1 ; c1ccc(-c2ccc3oc4cccc(-c5nc(-c6ccc(-c7ccc8ccccc8c7)cc6)nc(-c6ccc7ccccc7c6)n5)c4c3c2)cc1]

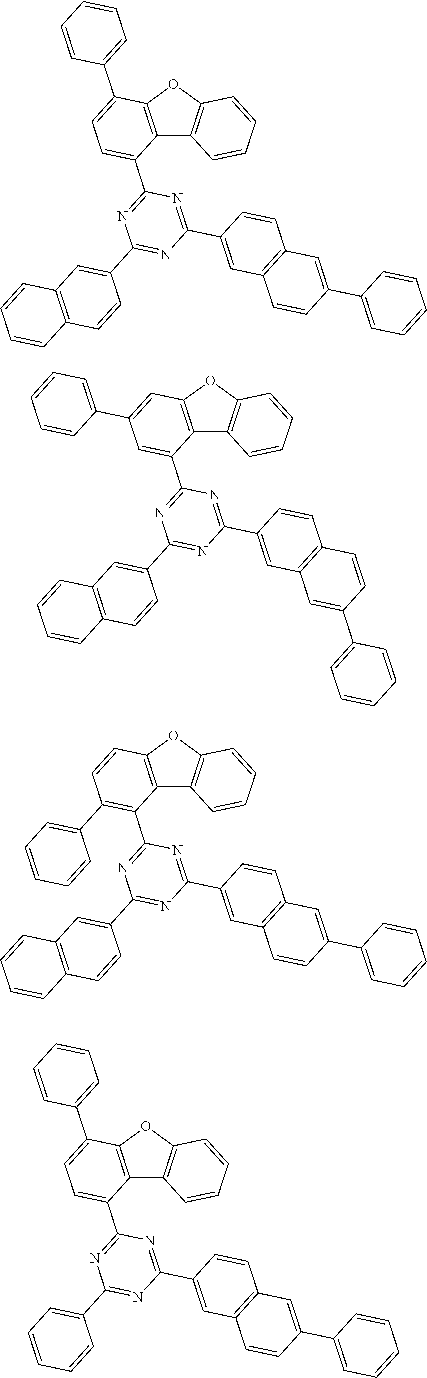

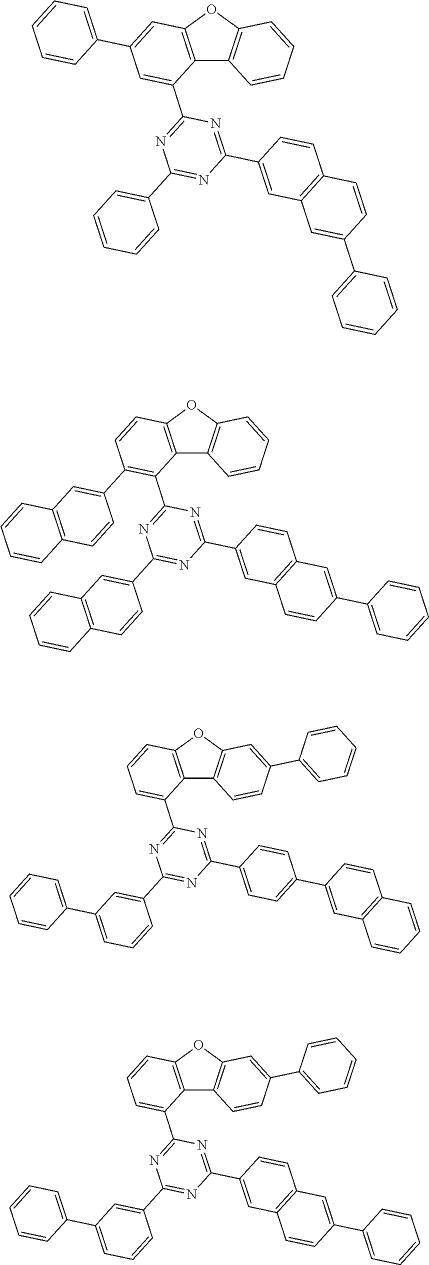 [SMILES: c1ccc(-c2ccc3ccc(-c4nc(-c5ccccc5)nc(-c5cc(-c6ccccc6)cc6oc7ccccc7c56)n4)cc3c2)cc1 ; c1ccc(-c2ccc3cc(-c4nc(-c5ccc6ccccc6c5)nc(-c5c(-c6ccc7ccccc7c6)ccc6oc7ccccc7c56)n4)ccc3c2)cc1 ; c1ccc(-c2cccc(-c3nc(-c4ccc(-c5ccc6ccccc6c5)cc4)nc(-c4cccc5oc6cc(-c7ccccc7)ccc6c45)n3)c2)cc1 ; c1ccc(-c2cccc(-c3nc(-c4ccc5cc(-c6ccccc6)ccc5c4)nc(-c4cccc5oc6cc(-c7ccccc7)ccc6c45)n3)c2)cc1]

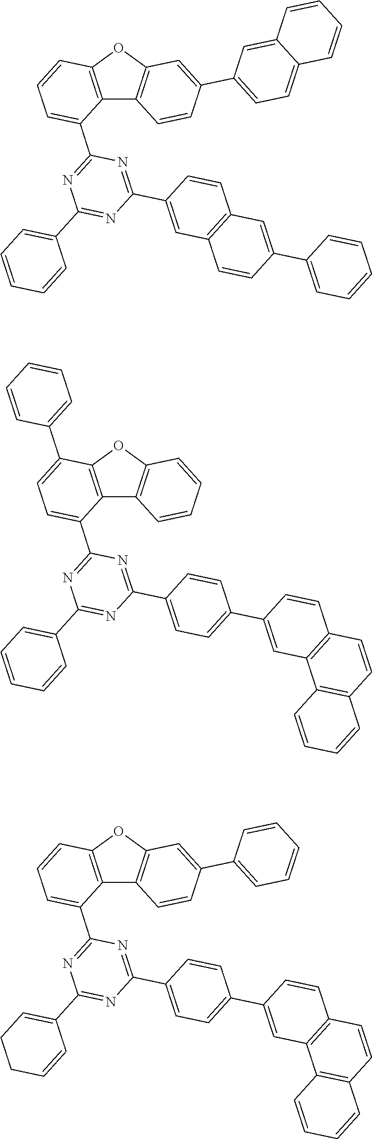 [SMILES: c1ccc(-c2ccc3cc(-c4nc(-c5ccccc5)nc(-c5cccc6oc7cc(-c8ccc9ccccc9c8)ccc7c56)n4)ccc3c2)cc1 ; c1ccc(-c2nc(-c3ccc(-c4ccc5ccc6ccccc6c5c4)cc3)nc(-c3ccc(-c4ccccc4)c4oc5ccccc5c34)n2)cc1 ; C1=CC(c2nc(-c3ccc(-c4ccc5ccc6ccccc6c5c4)cc3)nc(-c3cccc4oc5cc(-c6ccccc6)ccc5c34)n2)=CCC1]

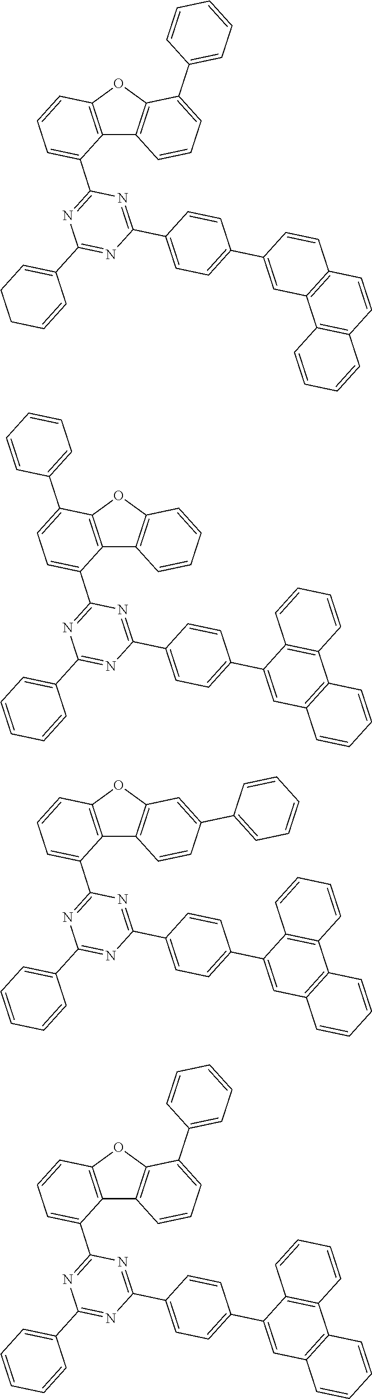 [SMILES: C1=CC(c2nc(-c3ccc(-c4ccc5ccc6ccccc6c5c4)cc3)nc(-c3cccc4oc5c(-c6ccccc6)cccc5c34)n2)=CCC1 ; c1ccc(-c2nc(-c3ccc(-c4cc5ccccc5c5ccccc45)cc3)nc(-c3ccc(-c4ccccc4)c4oc5ccccc5c34)n2)cc1 ; c1ccc(-c2ccc3c(c2)oc2cccc(-c4nc(-c5ccccc5)nc(-c5ccc(-c6cc7ccccc7c7ccccc67)cc5)n4)c23)cc1 ; c1ccc(-c2nc(-c3ccc(-c4cc5ccccc5c5ccccc45)cc3)nc(-c3cccc4oc5c(-c6ccccc6)cccc5c34)n2)cc1]

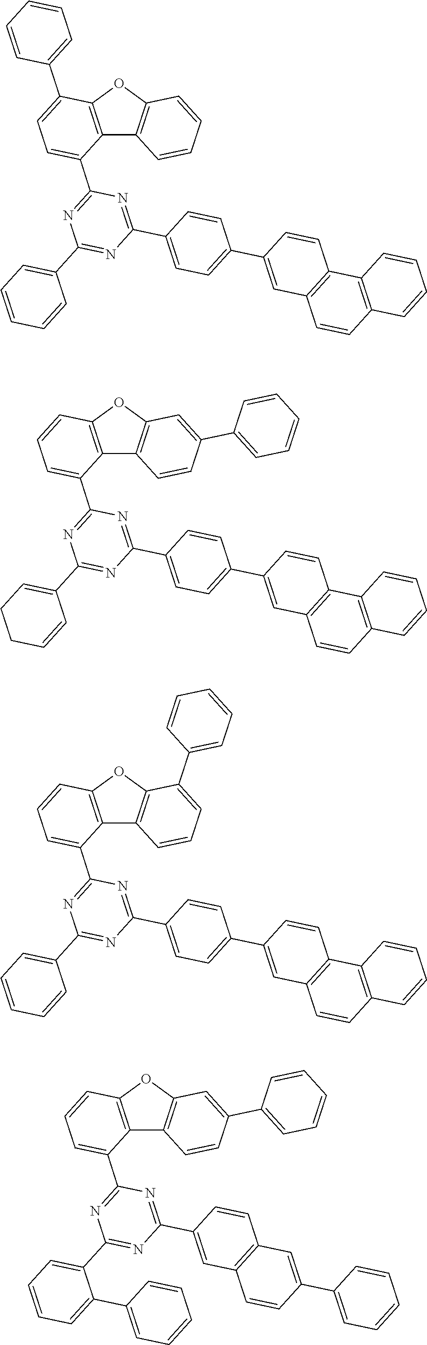 [SMILES: c1ccc(-c2nc(-c3ccc(-c4ccc5c(ccc6ccccc65)c4)cc3)nc(-c3ccc(-c4ccccc4)c4oc5ccccc5c34)n2)cc1 ; C1=CC(c2nc(-c3ccc(-c4ccc5c(ccc6ccccc65)c4)cc3)nc(-c3cccc4oc5cc(-c6ccccc6)ccc5c34)n2)=CCC1 ; c1ccc(-c2nc(-c3ccc(-c4ccc5c(ccc6ccccc65)c4)cc3)nc(-c3cccc4oc5c(-c6ccccc6)cccc5c34)n2)cc1 ; c1ccc(-c2ccc3cc(-c4nc(-c5ccccc5-c5ccccc5)nc(-c5cccc6oc7cc(-c8ccccc8)ccc7c56)n4)ccc3c2)cc1]

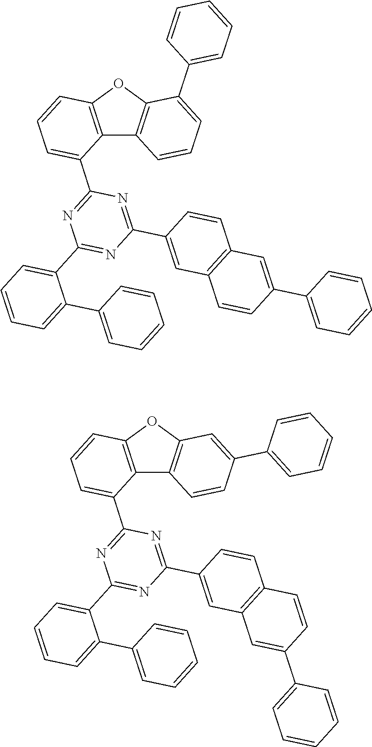 [SMILES: c1ccc(-c2ccc3cc(-c4nc(-c5ccccc5-c5ccccc5)nc(-c5cccc6oc7c(-c8ccccc8)cccc7c56)n4)ccc3c2)cc1 ; c1ccc(-c2ccc3ccc(-c4nc(-c5ccccc5-c5ccccc5)nc(-c5cccc6oc7cc(-c8ccccc8)ccc7c56)n4)cc3c2)cc1]

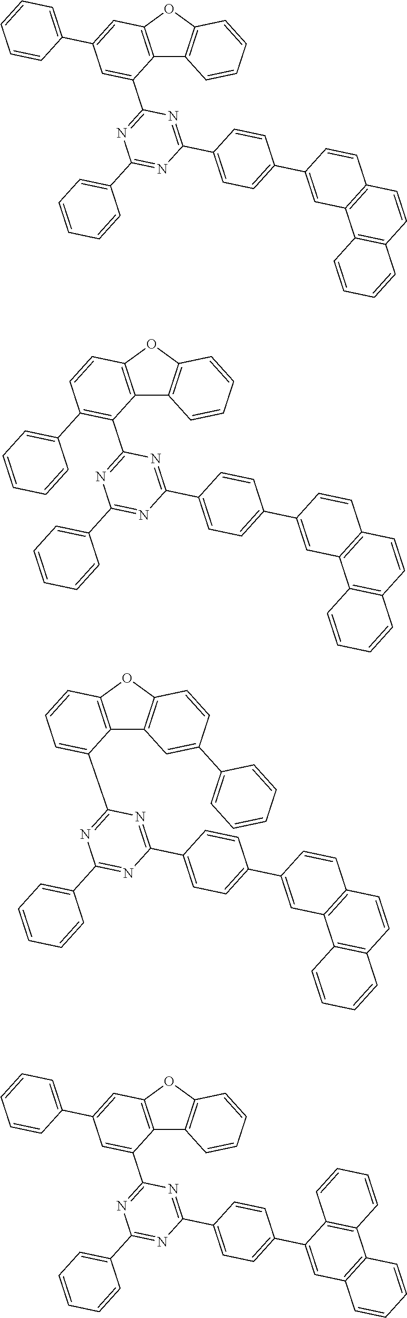 [SMILES: c1ccc(-c2cc(-c3nc(-c4ccccc4)nc(-c4ccc(-c5ccc6ccc7ccccc7c6c5)cc4)n3)c3c(c2)oc2ccccc23)cc1 ; c1ccc(-c2nc(-c3ccc(-c4ccc5ccc6ccccc6c5c4)cc3)nc(-c3c(-c4ccccc4)ccc4oc5ccccc5c34)n2)cc1 ; c1ccc(-c2ccc3oc4cccc(-c5nc(-c6ccccc6)nc(-c6ccc(-c7ccc8ccc9ccccc9c8c7)cc6)n5)c4c3c2)cc1 ; c1ccc(-c2cc(-c3nc(-c4ccccc4)nc(-c4ccc(-c5cc6ccccc6c6ccccc56)cc4)n3)c3c(c2)oc2ccccc23)cc1]

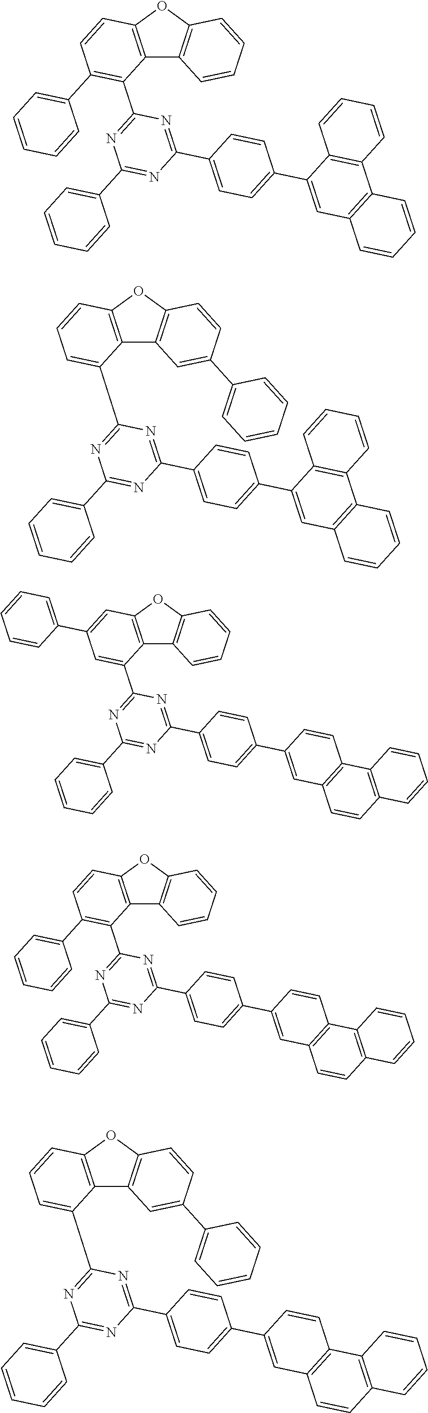

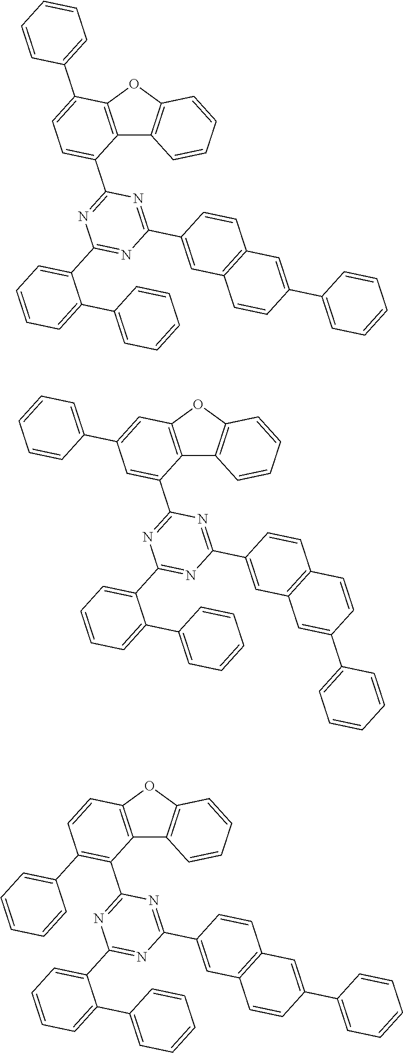 [SMILES: c1ccc(-c2ccc3cc(-c4nc(-c5ccccc5-c5ccccc5)nc(-c5ccc(-c6ccccc6)c6oc7ccccc7c56)n4)ccc3c2)cc1 ; c1ccc(-c2ccc3ccc(-c4nc(-c5ccccc5-c5ccccc5)nc(-c5cc(-c6ccccc6)cc6oc7ccccc7c56)n4)cc3c2)cc1 ; c1ccc(-c2ccc3cc(-c4nc(-c5ccccc5-c5ccccc5)nc(-c5c(-c6ccccc6)ccc6oc7ccccc7c56)n4)ccc3c2)cc1]

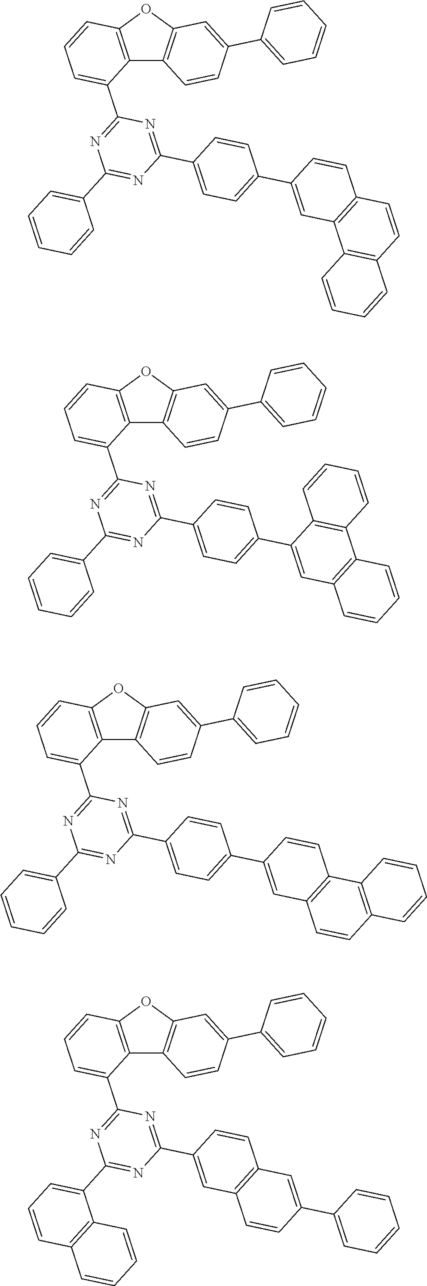 [SMILES: c1ccc(-c2ccc3c(c2)oc2cccc(-c4nc(-c5ccccc5)nc(-c5ccc(-c6ccc7ccc8ccccc8c7c6)cc5)n4)c23)cc1 ; c1ccc(-c2ccc3c(c2)oc2cccc(-c4nc(-c5ccccc5)nc(-c5ccc(-c6cc7ccccc7c7ccccc67)cc5)n4)c23)cc1 ; c1ccc(-c2ccc3c(c2)oc2cccc(-c4nc(-c5ccccc5)nc(-c5ccc(-c6ccc7c(ccc8ccccc87)c6)cc5)n4)c23)cc1 ; c1ccc(-c2ccc3cc(-c4nc(-c5cccc6ccccc56)nc(-c5cccc6oc7cc(-c8ccccc8)ccc7c56)n4)ccc3c2)cc1]

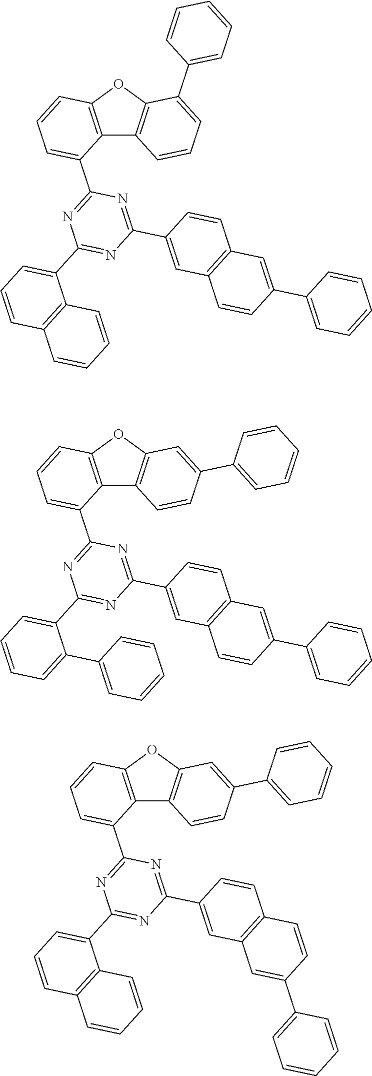 [SMILES: c1ccc(-c2ccc3cc(-c4nc(-c5cccc6ccccc56)nc(-c5cccc6oc7c(-c8ccccc8)cccc7c56)n4)ccc3c2)cc1 ; c1ccc(-c2ccc3cc(-c4nc(-c5ccccc5-c5ccccc5)nc(-c5cccc6oc7cc(-c8ccccc8)ccc7c56)n4)ccc3c2)cc1 ; c1ccc(-c2ccc3ccc(-c4nc(-c5cccc6ccccc56)nc(-c5cccc6oc7cc(-c8ccccc8)ccc7c56)n4)cc3c2)cc1]

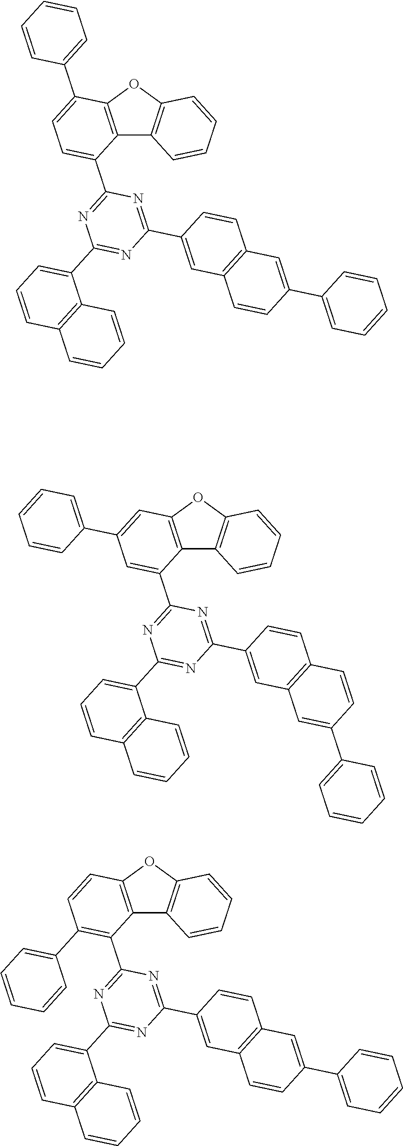 [SMILES: c1ccc(-c2ccc3cc(-c4nc(-c5cccc6ccccc56)nc(-c5ccc(-c6ccccc6)c6oc7ccccc7c56)n4)ccc3c2)cc1 ; c1ccc(-c2ccc3ccc(-c4nc(-c5cccc6ccccc56)nc(-c5cc(-c6ccccc6)cc6oc7ccccc7c56)n4)cc3c2)cc1 ; c1ccc(-c2ccc3cc(-c4nc(-c5cccc6ccccc56)nc(-c5c(-c6ccccc6)ccc6oc7ccccc7c56)n4)ccc3c2)cc1]

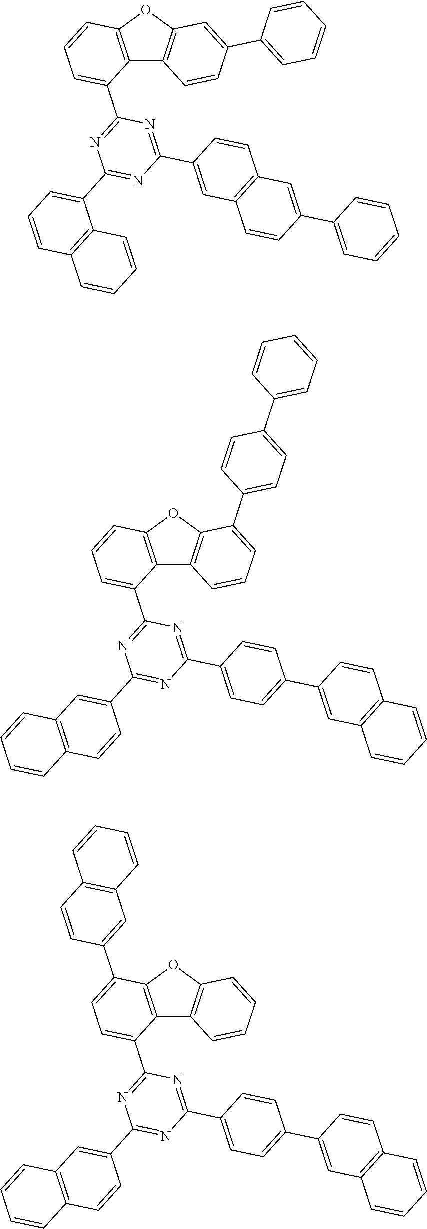 [SMILES: c1ccc(-c2ccc3cc(-c4nc(-c5cccc6ccccc56)nc(-c5cccc6oc7cc(-c8ccccc8)ccc7c56)n4)ccc3c2)cc1 ; c1ccc(-c2ccc(-c3cccc4c3oc3cccc(-c5nc(-c6ccc(-c7ccc8ccccc8c7)cc6)nc(-c6ccc7ccccc7c6)n5)c34)cc2)cc1 ; c1ccc2cc(-c3ccc(-c4nc(-c5ccc6ccccc6c5)nc(-c5ccc(-c6ccc7ccccc7c6)c6oc7ccccc7c56)n4)cc3)ccc2c1]

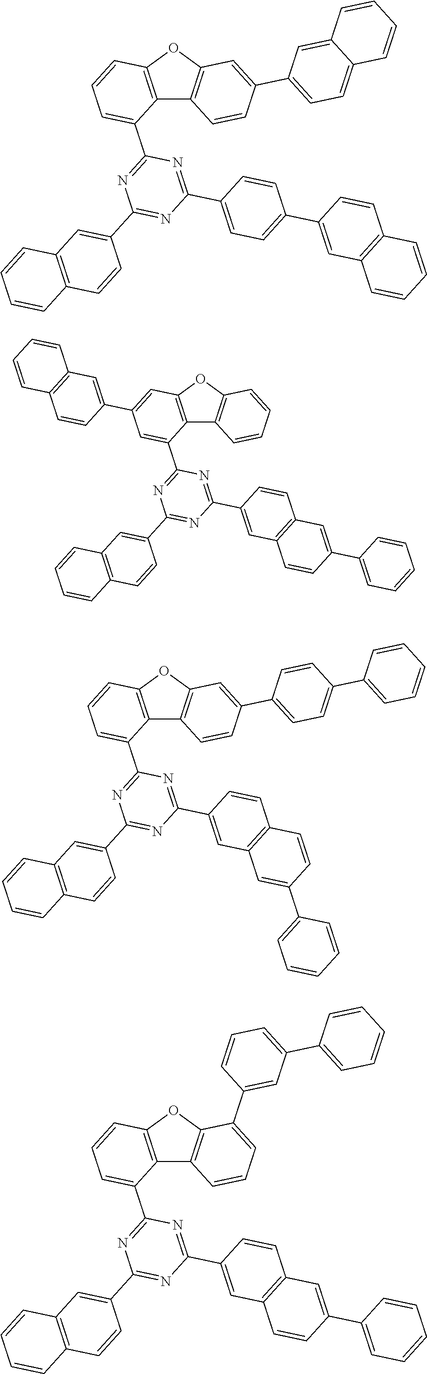

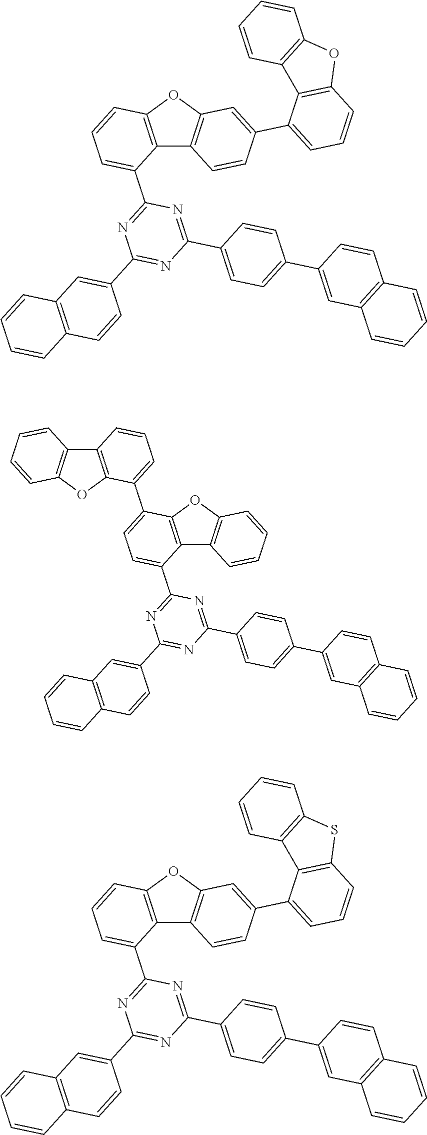 [SMILES: c1ccc2cc(-c3ccc(-c4nc(-c5ccc6ccccc6c5)nc(-c5cccc6oc7cc(-c8cccc9oc%10ccccc%10c89)ccc7c56)n4)cc3)ccc2c1 ; c1ccc2cc(-c3ccc(-c4nc(-c5ccc6ccccc6c5)nc(-c5ccc(-c6cccc7c6oc6ccccc67)c6oc7ccccc7c56)n4)cc3)ccc2c1 ; c1ccc2cc(-c3ccc(-c4nc(-c5ccc6ccccc6c5)nc(-c5cccc6oc7cc(-c8cccc9sc%10ccccc%10c89)ccc7c56)n4)cc3)ccc2c1]

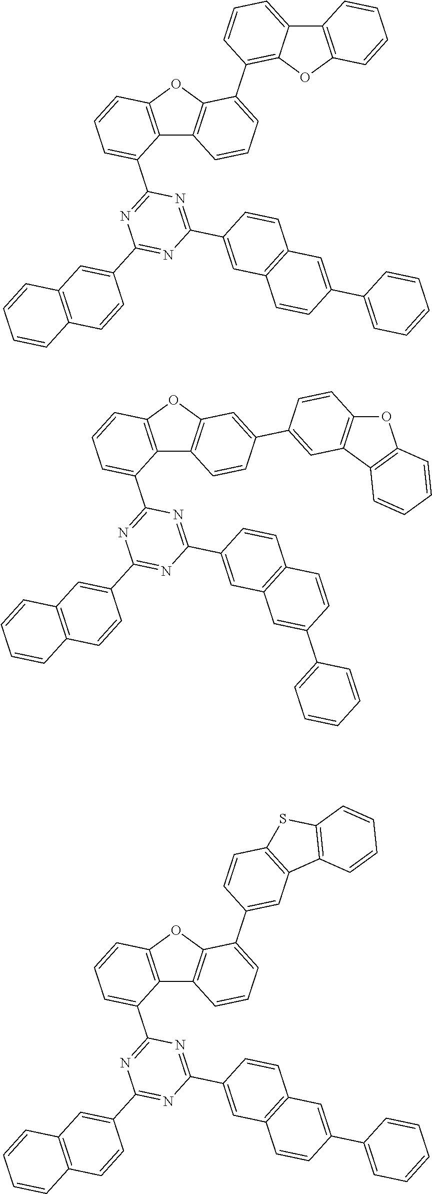 [SMILES: c1ccc(-c2ccc3cc(-c4nc(-c5ccc6ccccc6c5)nc(-c5cccc6oc7c(-c8cccc9c8oc8ccccc89)cccc7c56)n4)ccc3c2)cc1 ; c1ccc(-c2ccc3ccc(-c4nc(-c5ccc6ccccc6c5)nc(-c5cccc6oc7cc(-c8ccc9oc%10ccccc%10c9c8)ccc7c56)n4)cc3c2)cc1 ; c1ccc(-c2ccc3cc(-c4nc(-c5ccc6ccccc6c5)nc(-c5cccc6oc7c(-c8ccc9sc%10ccccc%10c9c8)cccc7c56)n4)ccc3c2)cc1]

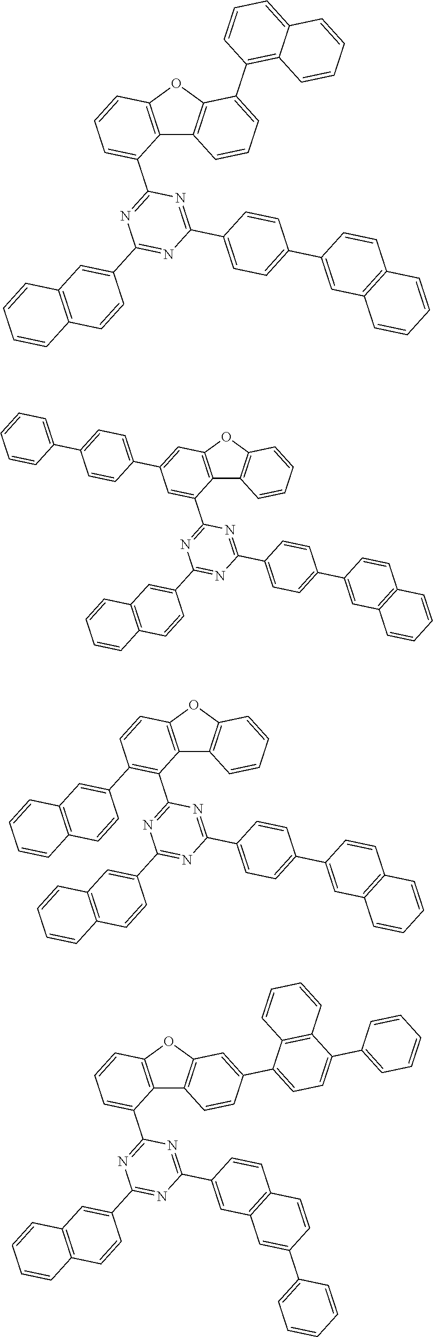 [SMILES: c1ccc2cc(-c3ccc(-c4nc(-c5ccc6ccccc6c5)nc(-c5cccc6oc7c(-c8cccc9ccccc89)cccc7c56)n4)cc3)ccc2c1 ; c1ccc(-c2ccc(-c3cc(-c4nc(-c5ccc(-c6ccc7ccccc7c6)cc5)nc(-c5ccc6ccccc6c5)n4)c4c(c3)oc3ccccc34)cc2)cc1 ; c1ccc2cc(-c3ccc(-c4nc(-c5ccc6ccccc6c5)nc(-c5c(-c6ccc7ccccc7c6)ccc6oc7ccccc7c56)n4)cc3)ccc2c1 ; c1ccc(-c2ccc3ccc(-c4nc(-c5ccc6ccccc6c5)nc(-c5cccc6oc7cc(-c8ccc(-c9ccccc9)c9ccccc89)ccc7c56)n4)cc3c2)cc1]

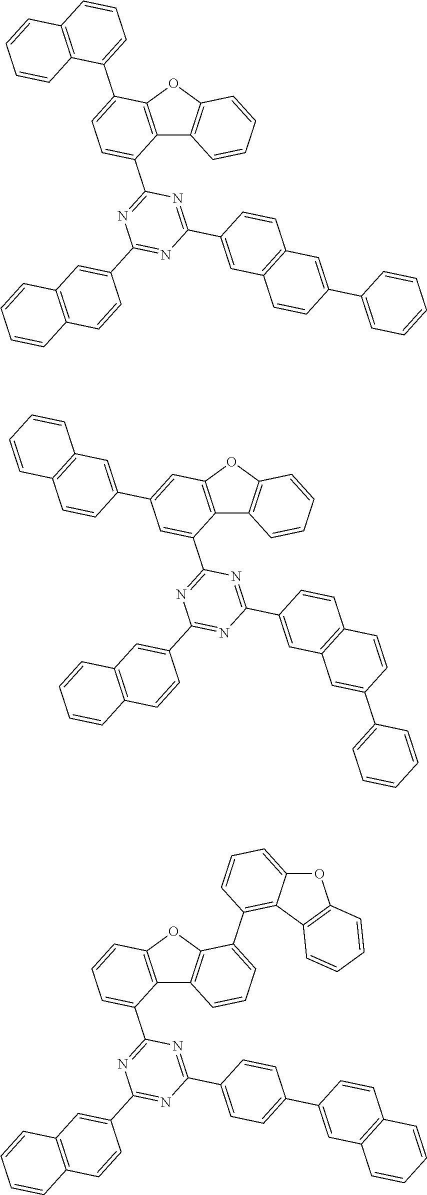 [SMILES: c1ccc(-c2ccc3cc(-c4nc(-c5ccc6ccccc6c5)nc(-c5ccc(-c6cccc7ccccc67)c6oc7ccccc7c56)n4)ccc3c2)cc1 ; c1ccc(-c2ccc3ccc(-c4nc(-c5ccc6ccccc6c5)nc(-c5cc(-c6ccc7ccccc7c6)cc6oc7ccccc7c56)n4)cc3c2)cc1 ; c1ccc2cc(-c3ccc(-c4nc(-c5ccc6ccccc6c5)nc(-c5cccc6oc7c(-c8cccc9oc%10ccccc%10c89)cccc7c56)n4)cc3)ccc2c1]

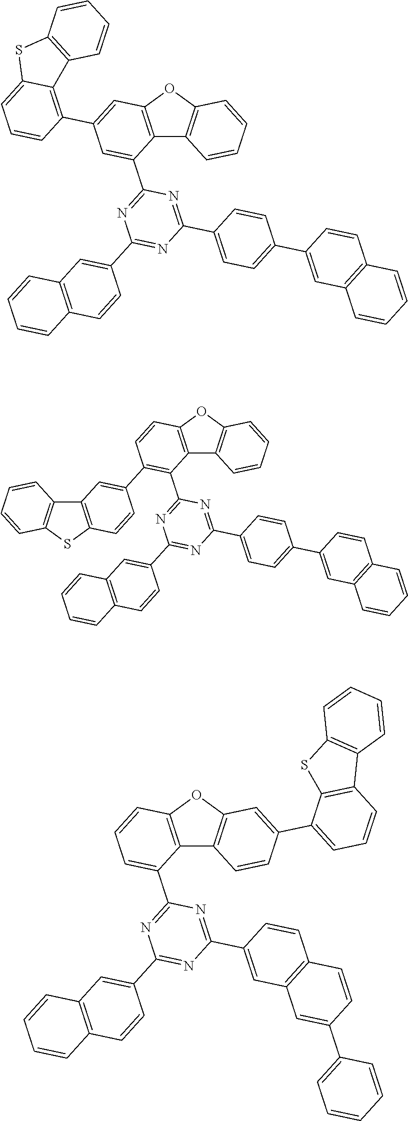 [SMILES: c1ccc2cc(-c3ccc(-c4nc(-c5ccc6ccccc6c5)nc(-c5cc(-c6cccc7sc8ccccc8c67)cc6oc7ccccc7c56)n4)cc3)ccc2c1 ; c1ccc2cc(-c3ccc(-c4nc(-c5ccc6ccccc6c5)nc(-c5c(-c6ccc7sc8ccccc8c7c6)ccc6oc7ccccc7c56)n4)cc3)ccc2c1 ; c1ccc(-c2ccc3ccc(-c4nc(-c5ccc6ccccc6c5)nc(-c5cccc6oc7cc(-c8cccc9c8sc8ccccc89)ccc7c56)n4)cc3c2)cc1]

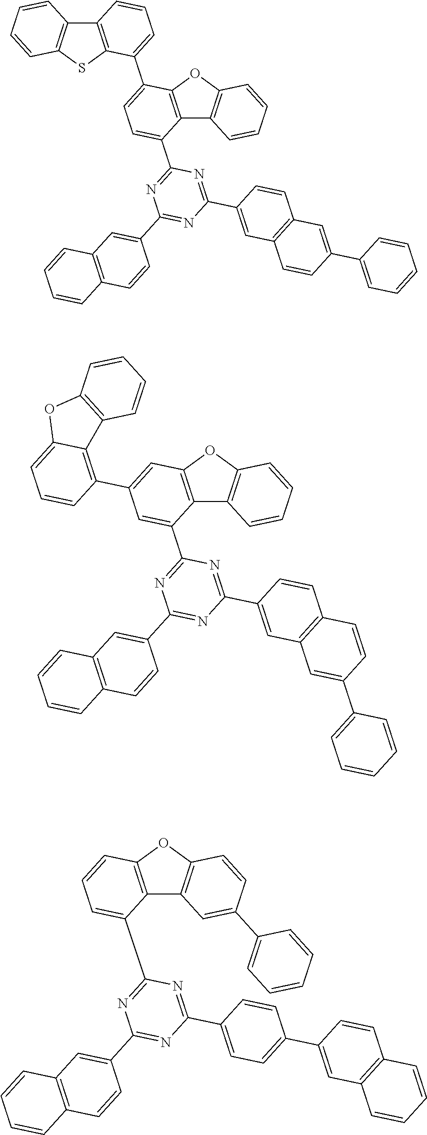 [SMILES: c1ccc(-c2ccc3cc(-c4nc(-c5ccc6ccccc6c5)nc(-c5ccc(-c6cccc7c6sc6ccccc67)c6oc7ccccc7c56)n4)ccc3c2)cc1 ; c1ccc(-c2ccc3ccc(-c4nc(-c5ccc6ccccc6c5)nc(-c5cc(-c6cccc7oc8ccccc8c67)cc6oc7ccccc7c56)n4)cc3c2)cc1 ; c1ccc(-c2ccc3oc4cccc(-c5nc(-c6ccc(-c7ccc8ccccc8c7)cc6)nc(-c6ccc7ccccc7c6)n5)c4c3c2)cc1]

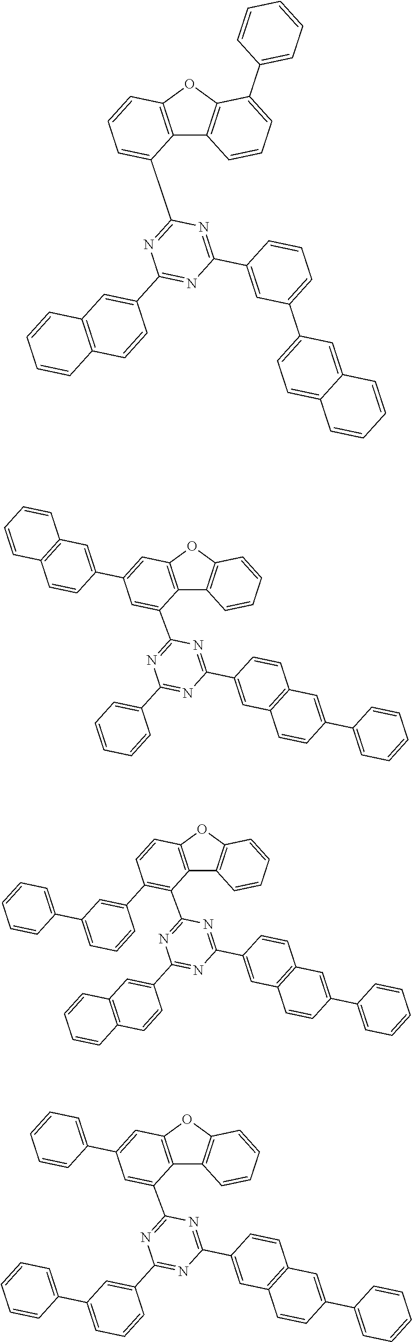 [SMILES: c1ccc(-c2cccc3c2oc2cccc(-c4nc(-c5cccc(-c6ccc7ccccc7c6)c5)nc(-c5ccc6ccccc6c5)n4)c23)cc1 ; c1ccc(-c2ccc3cc(-c4nc(-c5ccccc5)nc(-c5cc(-c6ccc7ccccc7c6)cc6oc7ccccc7c56)n4)ccc3c2)cc1 ; c1ccc(-c2cccc(-c3ccc4oc5ccccc5c4c3-c3nc(-c4ccc5ccccc5c4)nc(-c4ccc5cc(-c6ccccc6)ccc5c4)n3)c2)cc1 ; c1ccc(-c2cccc(-c3nc(-c4ccc5cc(-c6ccccc6)ccc5c4)nc(-c4cc(-c5ccccc5)cc5oc6ccccc6c45)n3)c2)cc1]

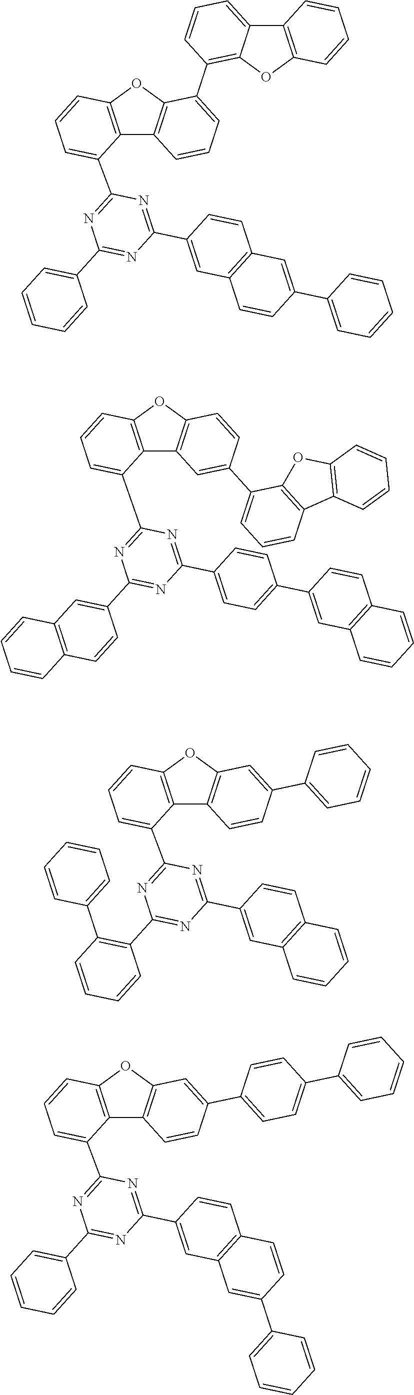 [SMILES: c1ccc(-c2ccc3cc(-c4nc(-c5ccccc5)nc(-c5cccc6oc7c(-c8cccc9c8oc8ccccc89)cccc7c56)n4)ccc3c2)cc1 ; c1ccc2cc(-c3ccc(-c4nc(-c5ccc6ccccc6c5)nc(-c5cccc6oc7ccc(-c8cccc9c8oc8ccccc89)cc7c56)n4)cc3)ccc2c1 ; c1ccc(-c2ccc3c(c2)oc2cccc(-c4nc(-c5ccc6ccccc6c5)nc(-c5ccccc5-c5ccccc5)n4)c23)cc1 ; c1ccc(-c2ccc(-c3ccc4c(c3)oc3cccc(-c5nc(-c6ccccc6)nc(-c6ccc7ccc(-c8ccccc8)cc7c6)n5)c34)cc2)cc1]

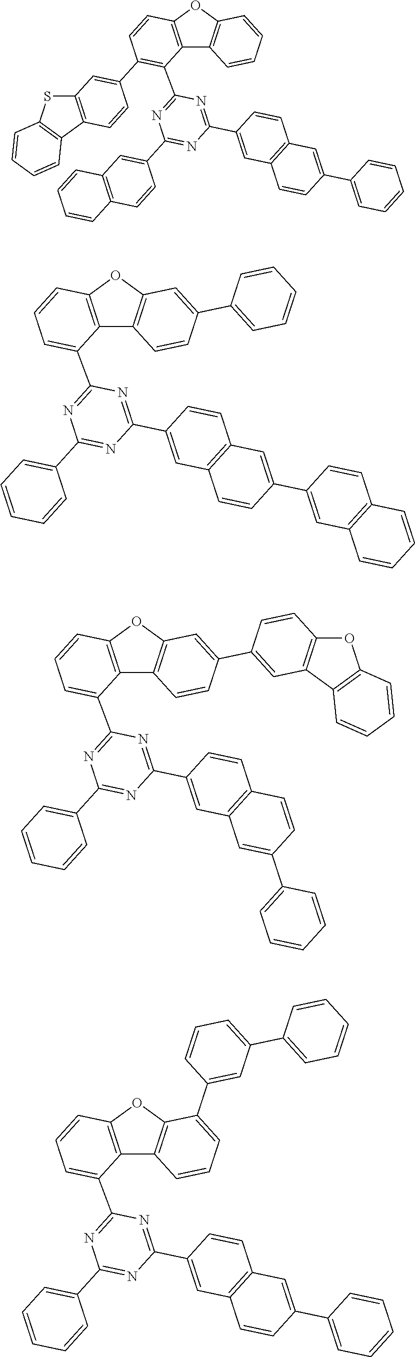 [SMILES: c1ccc(-c2ccc3cc(-c4nc(-c5ccc6ccccc6c5)nc(-c5c(-c6ccc7c(c6)sc6ccccc67)ccc6oc7ccccc7c56)n4)ccc3c2)cc1 ; c1ccc(-c2ccc3c(c2)oc2cccc(-c4nc(-c5ccccc5)nc(-c5ccc6cc(-c7ccc8ccccc8c7)ccc6c5)n4)c23)cc1 ; c1ccc(-c2ccc3ccc(-c4nc(-c5ccccc5)nc(-c5cccc6oc7cc(-c8ccc9oc%10ccccc%10c9c8)ccc7c56)n4)cc3c2)cc1 ; c1ccc(-c2cccc(-c3cccc4c3oc3cccc(-c5nc(-c6ccccc6)nc(-c6ccc7cc(-c8ccccc8)ccc7c6)n5)c34)c2)cc1]

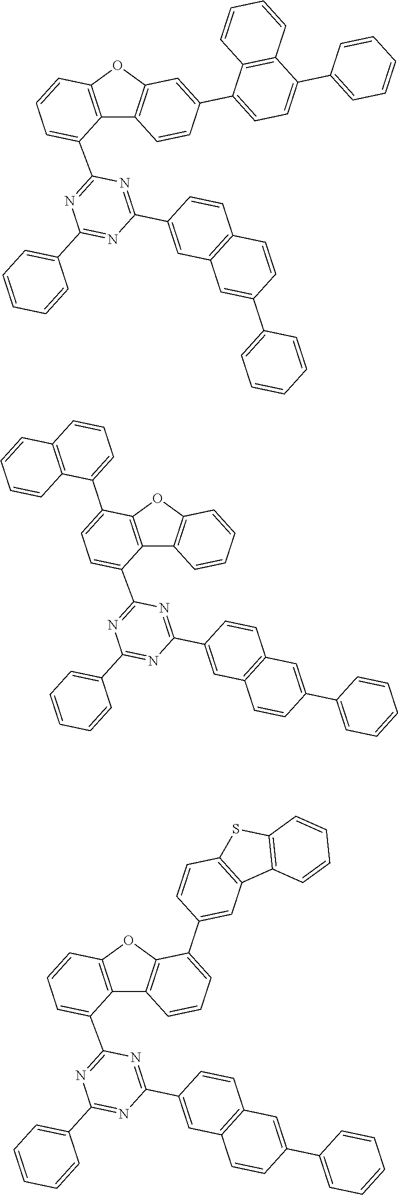 [SMILES: c1ccc(-c2ccc3ccc(-c4nc(-c5ccccc5)nc(-c5cccc6oc7cc(-c8ccc(-c9ccccc9)c9ccccc89)ccc7c56)n4)cc3c2)cc1 ; c1ccc(-c2ccc3cc(-c4nc(-c5ccccc5)nc(-c5ccc(-c6cccc7ccccc67)c6oc7ccccc7c56)n4)ccc3c2)cc1 ; c1ccc(-c2ccc3cc(-c4nc(-c5ccccc5)nc(-c5cccc6oc7c(-c8ccc9sc%10ccccc%10c9c8)cccc7c56)n4)ccc3c2)cc1]

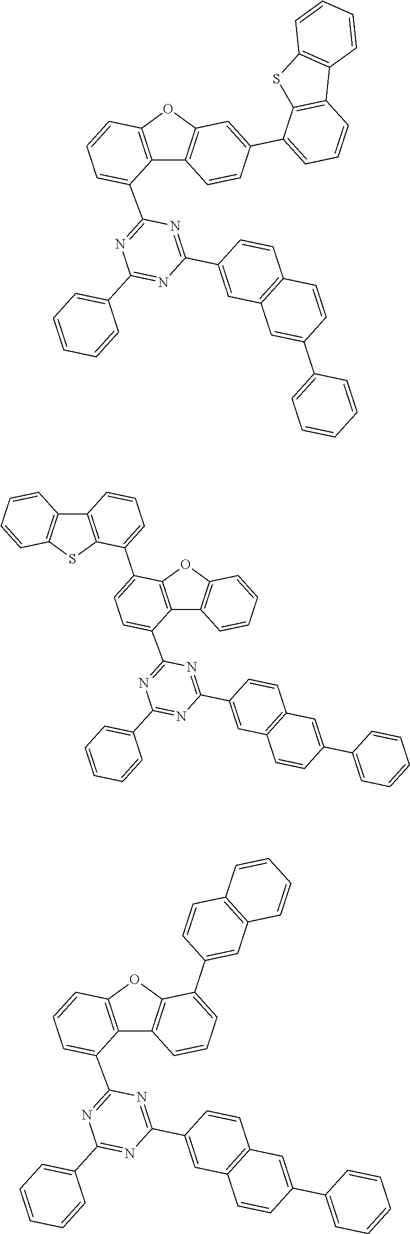 [SMILES: c1ccc(-c2ccc3ccc(-c4nc(-c5ccccc5)nc(-c5cccc6oc7cc(-c8cccc9c8sc8ccccc89)ccc7c56)n4)cc3c2)cc1 ; c1ccc(-c2ccc3cc(-c4nc(-c5ccccc5)nc(-c5ccc(-c6cccc7c6sc6ccccc67)c6oc7ccccc7c56)n4)ccc3c2)cc1 ; c1ccc(-c2ccc3cc(-c4nc(-c5ccccc5)nc(-c5cccc6oc7c(-c8ccc9ccccc9c8)cccc7c56)n4)ccc3c2)cc1]

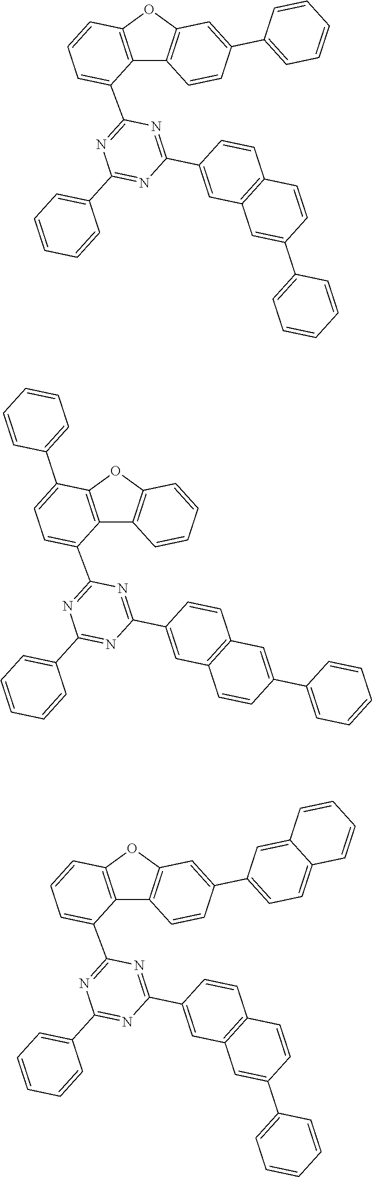 [SMILES: c1ccc(-c2ccc3ccc(-c4nc(-c5ccccc5)nc(-c5cccc6oc7cc(-c8ccccc8)ccc7c56)n4)cc3c2)cc1 ; c1ccc(-c2ccc3cc(-c4nc(-c5ccccc5)nc(-c5ccc(-c6ccccc6)c6oc7ccccc7c56)n4)ccc3c2)cc1 ; c1ccc(-c2ccc3ccc(-c4nc(-c5ccccc5)nc(-c5cccc6oc7cc(-c8ccc9ccccc9c8)ccc7c56)n4)cc3c2)cc1]

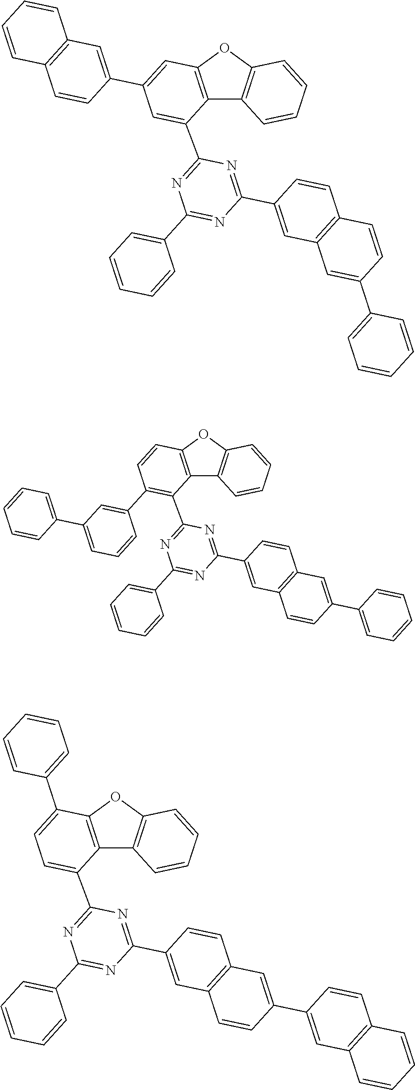 [SMILES: c1ccc(-c2ccc3ccc(-c4nc(-c5ccccc5)nc(-c5cc(-c6ccc7ccccc7c6)cc6oc7ccccc7c56)n4)cc3c2)cc1 ; c1ccc(-c2cccc(-c3ccc4oc5ccccc5c4c3-c3nc(-c4ccccc4)nc(-c4ccc5cc(-c6ccccc6)ccc5c4)n3)c2)cc1 ; c1ccc(-c2nc(-c3ccc4cc(-c5ccc6ccccc6c5)ccc4c3)nc(-c3ccc(-c4ccccc4)c4oc5ccccc5c34)n2)cc1]

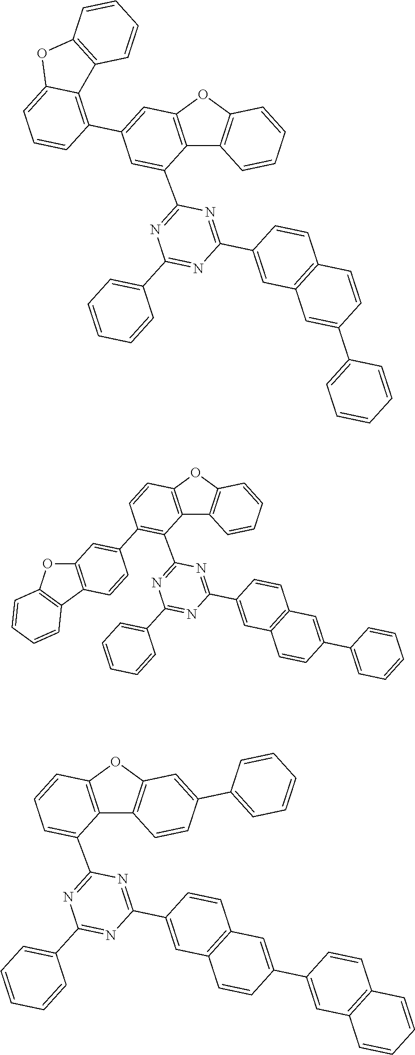 [SMILES: c1ccc(-c2ccc3ccc(-c4nc(-c5ccccc5)nc(-c5cc(-c6cccc7oc8ccccc8c67)cc6oc7ccccc7c56)n4)cc3c2)cc1 ; c1ccc(-c2ccc3cc(-c4nc(-c5ccccc5)nc(-c5c(-c6ccc7c(c6)oc6ccccc67)ccc6oc7ccccc7c56)n4)ccc3c2)cc1 ; c1ccc(-c2ccc3c(c2)oc2cccc(-c4nc(-c5ccccc5)nc(-c5ccc6cc(-c7ccc8ccccc8c7)ccc6c5)n4)c23)cc1]

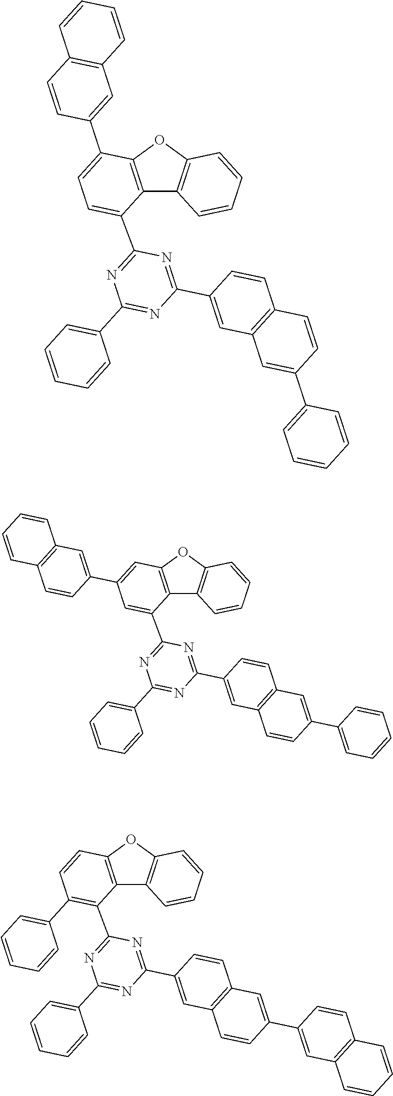 [SMILES: c1ccc(-c2ccc3ccc(-c4nc(-c5ccccc5)nc(-c5ccc(-c6ccc7ccccc7c6)c6oc7ccccc7c56)n4)cc3c2)cc1 ; c1ccc(-c2ccc3cc(-c4nc(-c5ccccc5)nc(-c5cc(-c6ccc7ccccc7c6)cc6oc7ccccc7c56)n4)ccc3c2)cc1 ; c1ccc(-c2nc(-c3ccc4cc(-c5ccc6ccccc6c5)ccc4c3)nc(-c3c(-c4ccccc4)ccc4oc5ccccc5c34)n2)cc1]

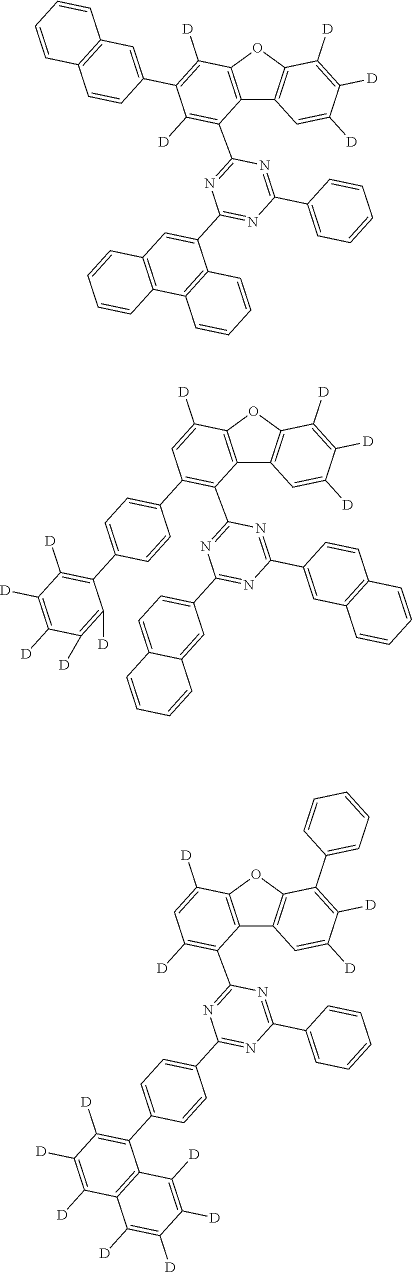 [SMILES: [2H]c1cc2c(oc3c([2H])c(-c4ccc5ccccc5c4)c([2H])c(-c4nc(-c5ccccc5)nc(-c5cc6ccccc6c6ccccc56)n4)c32)c([2H])c1[2H] ; [2H]c1cc2c(oc3c([2H])cc(-c4ccc(-c5c([2H])c([2H])c([2H])c([2H])c5[2H])cc4)c(-c4nc(-c5ccc6ccccc6c5)nc(-c5ccc6ccccc6c5)n4)c32)c([2H])c1[2H] ; [2H]c1cc2c(oc3c([2H])cc([2H])c(-c4nc(-c5ccccc5)nc(-c5ccc(-c6c([2H])c([2H])c([2H])c7c([2H])c([2H])c([2H])c([2H])c67)cc5)n4)c32)c(-c2ccccc2)c1[2H]]

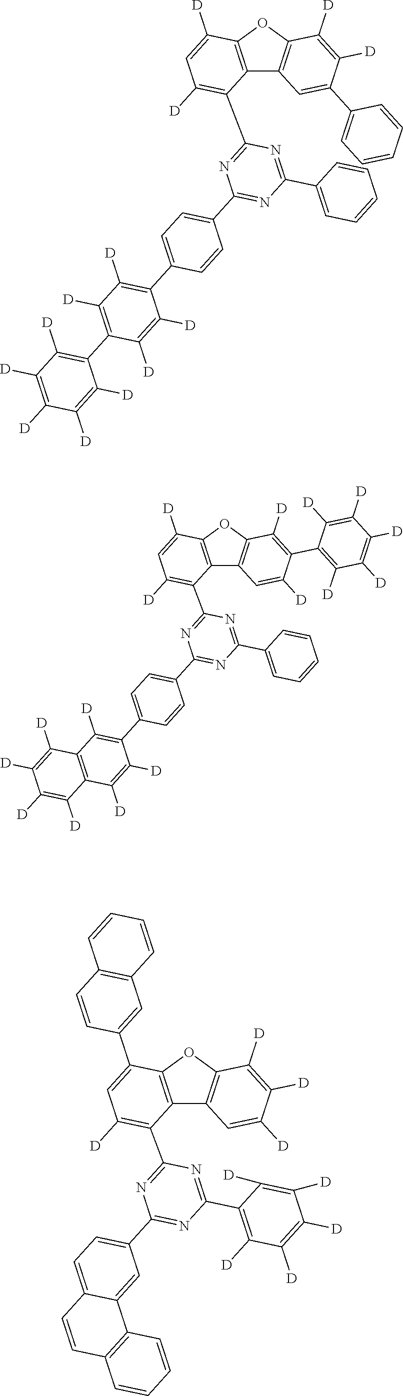 [SMILES: [2H]c1cc([2H])c2oc3c([2H])c([2H])c(-c4ccccc4)cc3c2c1-c1nc(-c2ccccc2)nc(-c2ccc(-c3c([2H])c([2H])c(-c4c([2H])c([2H])c([2H])c([2H])c4[2H])c([2H])c3[2H])cc2)n1 ; [2H]c1cc2c(oc3c([2H])cc([2H])c(-c4nc(-c5ccccc5)nc(-c5ccc(-c6c([2H])c([2H])c7c([2H])c([2H])c([2H])c([2H])c7c6[2H])cc5)n4)c32)c([2H])c1-c1c([2H])c([2H])c([2H])c([2H])c1[2H] ; [2H]c1cc2c(oc3c(-c4ccc5ccccc5c4)cc([2H])c(-c4nc(-c5ccc6ccc7ccccc7c6c5)nc(-c5c([2H])c([2H])c([2H])c([2H])c5[2H])n4)c32)c([2H])c1[2H]]

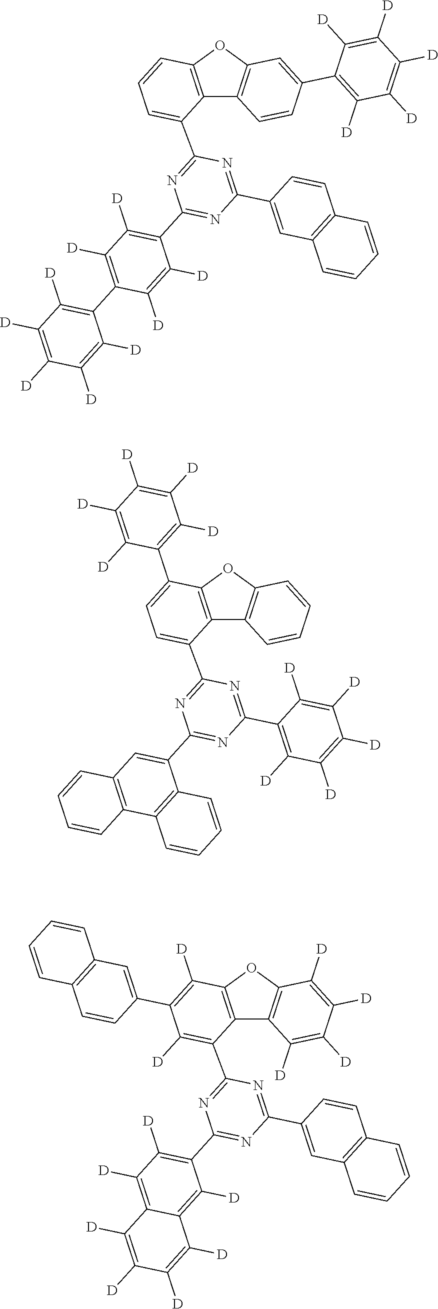 [SMILES: [2H]c1c([2H])c([2H])c(-c2ccc3c(c2)oc2cccc(-c4nc(-c5ccc6ccccc6c5)nc(-c5c([2H])c([2H])c(-c6c([2H])c([2H])c([2H])c([2H])c6[2H])c([2H])c5[2H])n4)c23)c([2H])c1[2H] ; [2H]c1c([2H])c([2H])c(-c2nc(-c3cc4ccccc4c4ccccc34)nc(-c3ccc(-c4c([2H])c([2H])c([2H])c([2H])c4[2H])c4oc5ccccc5c34)n2)c([2H])c1[2H] ; [2H]c1c([2H])c([2H])c2c(oc3c([2H])c(-c4ccc5ccccc5c4)c([2H])c(-c4nc(-c5ccc6ccccc6c5)nc(-c5c([2H])c([2H])c6c([2H])c([2H])c([2H])c([2H])c6c5[2H])n4)c32)c1[2H]]

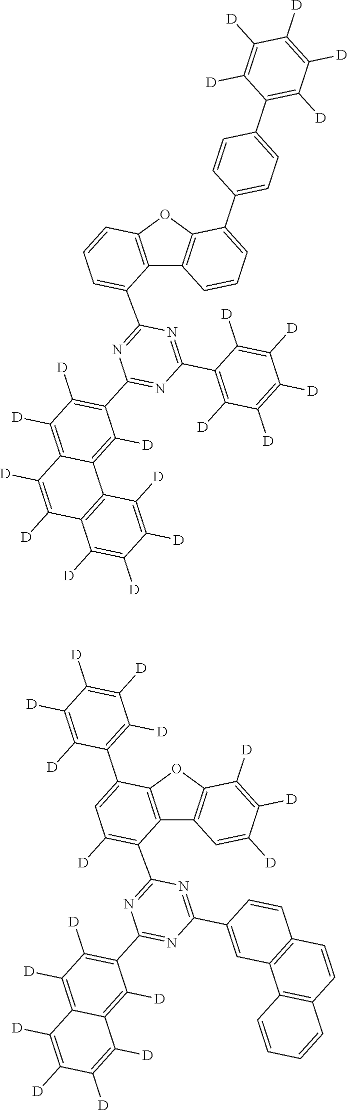 [SMILES: [2H]c1c([2H])c([2H])c(-c2ccc(-c3cccc4c3oc3cccc(-c5nc(-c6c([2H])c([2H])c([2H])c([2H])c6[2H])nc(-c6c([2H])c([2H])c7c([2H])c([2H])c8c([2H])c([2H])c([2H])c([2H])c8c7c6[2H])n5)c34)cc2)c([2H])c1[2H] ; [2H]c1cc2c(oc3c(-c4c([2H])c([2H])c([2H])c([2H])c4[2H])cc([2H])c(-c4nc(-c5ccc6ccc7ccccc7c6c5)nc(-c5c([2H])c([2H])c6c([2H])c([2H])c([2H])c([2H])c6c5[2H])n4)c32)c([2H])c1[2H]]

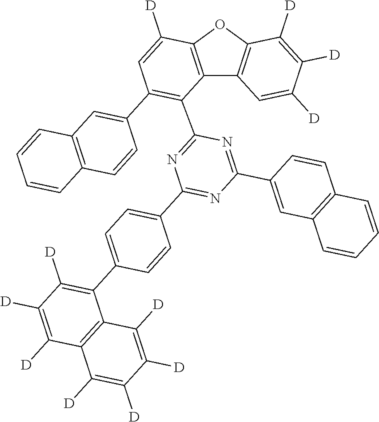 [SMILES: [2H]c1cc2c(oc3c([2H])cc(-c4ccc5ccccc5c4)c(-c4nc(-c5ccc(-c6c([2H])c([2H])c([2H])c7c([2H])c([2H])c([2H])c([2H])c67)cc5)nc(-c5ccc6ccccc6c5)n4)c32)c([2H])c1[2H]]

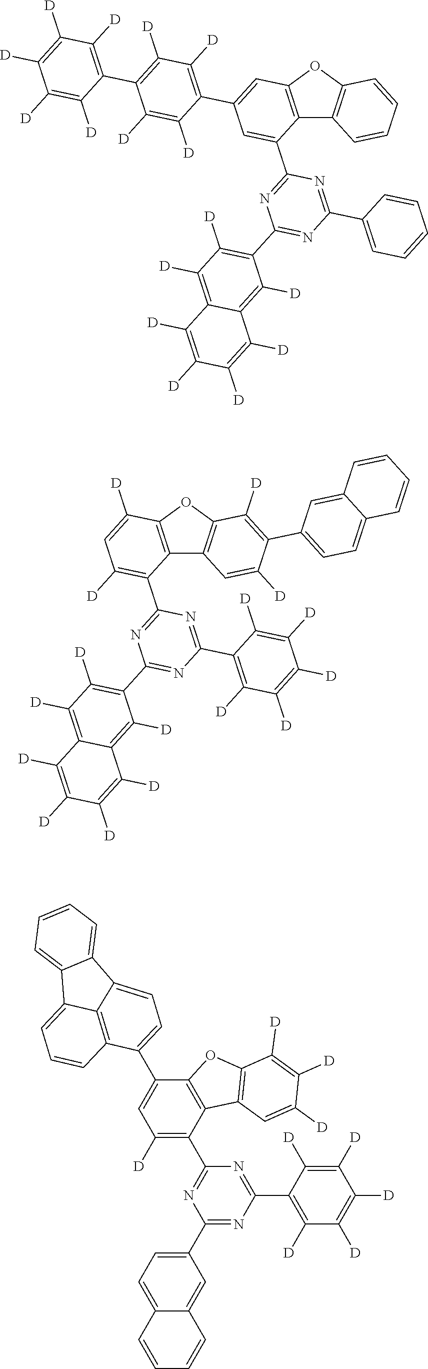 [SMILES: [2H]c1c([2H])c([2H])c(-c2c([2H])c([2H])c(-c3cc(-c4nc(-c5ccccc5)nc(-c5c([2H])c([2H])c6c([2H])c([2H])c([2H])c([2H])c6c5[2H])n4)c4c(c3)oc3ccccc34)c([2H])c2[2H])c([2H])c1[2H] ; [2H]c1cc2c(oc3c([2H])cc([2H])c(-c4nc(-c5c([2H])c([2H])c([2H])c([2H])c5[2H])nc(-c5c([2H])c([2H])c6c([2H])c([2H])c([2H])c([2H])c6c5[2H])n4)c32)c([2H])c1-c1ccc2ccccc2c1 ; [2H]c1cc2c(oc3c(-c4ccc5c6c(cccc46)-c4ccccc4-5)cc([2H])c(-c4nc(-c5ccc6ccccc6c5)nc(-c5c([2H])c([2H])c([2H])c([2H])c5[2H])n4)c32)c([2H])c1[2H]]

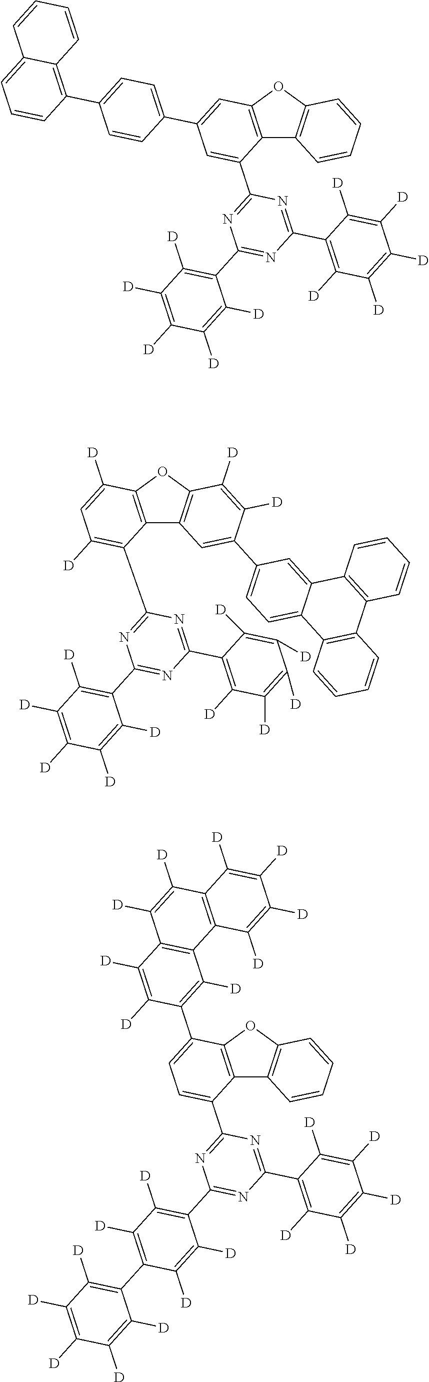

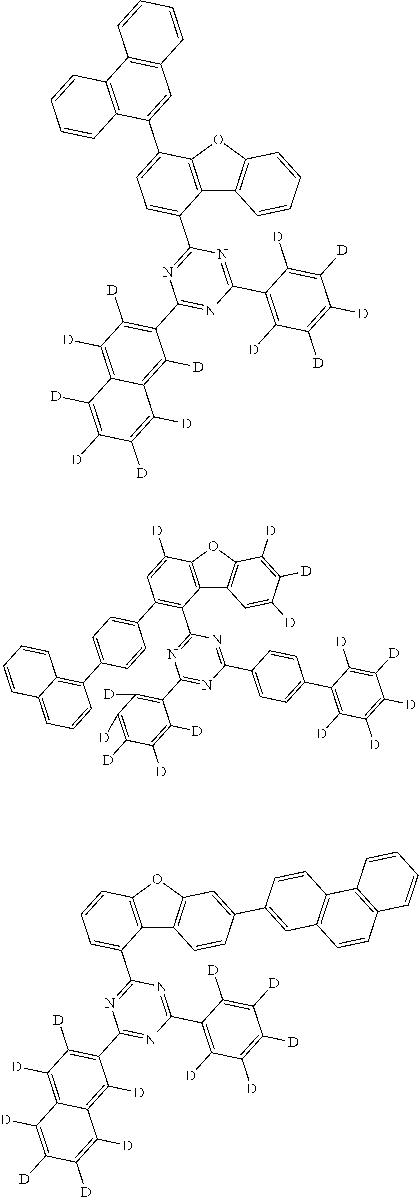 [SMILES: [2H]c1c([2H])c([2H])c(-c2nc(-c3c([2H])c([2H])c4c([2H])c([2H])c([2H])c([2H])c4c3[2H])nc(-c3ccc(-c4cc5ccccc5c5ccccc45)c4oc5ccccc5c34)n2)c([2H])c1[2H] ; [2H]c1cc2c(oc3c([2H])cc(-c4ccc(-c5cccc6ccccc56)cc4)c(-c4nc(-c5ccc(-c6c([2H])c([2H])c([2H])c([2H])c6[2H])cc5)nc(-c5c([2H])c([2H])c([2H])c([2H])c5[2H])n4)c32)c([2H])c1[2H] ; [2H]c1c([2H])c([2H])c(-c2nc(-c3c([2H])c([2H])c4c([2H])c([2H])c([2H])c([2H])c4c3[2H])nc(-c3cccc4oc5cc(-c6ccc7c(ccc8ccccc87)c6)ccc5c34)n2)c([2H])c1[2H]]

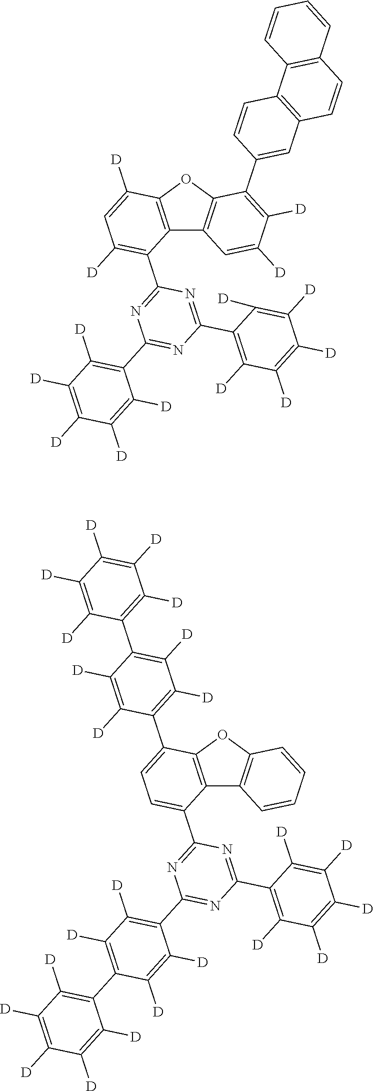 [SMILES: [2H]c1cc2c(oc3c([2H])cc([2H])c(-c4nc(-c5c([2H])c([2H])c([2H])c([2H])c5[2H])nc(-c5c([2H])c([2H])c([2H])c([2H])c5[2H])n4)c32)c(-c2ccc3c(ccc4ccccc43)c2)c1[2H] ; [2H]c1c([2H])c([2H])c(-c2nc(-c3c([2H])c([2H])c(-c4c([2H])c([2H])c([2H])c([2H])c4[2H])c([2H])c3[2H])nc(-c3ccc(-c4c([2H])c([2H])c(-c5c([2H])c([2H])c([2H])c([2H])c5[2H])c([2H])c4[2H])c4oc5ccccc5c34)n2)c([2H])c1[2H]]

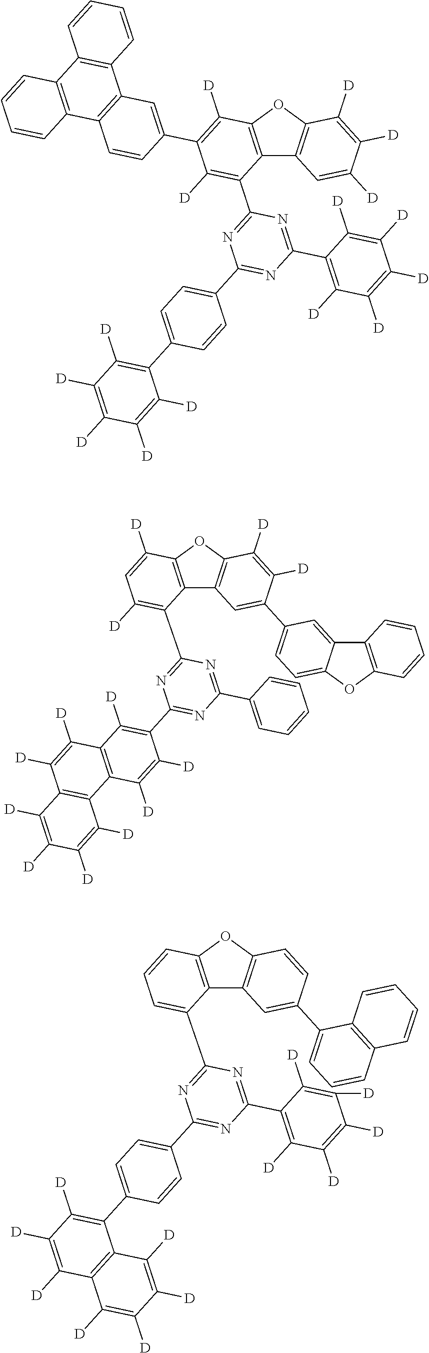 [SMILES: [2H]c1cc2c(oc3c([2H])c(-c4ccc5c6ccccc6c6ccccc6c5c4)c([2H])c(-c4nc(-c5ccc(-c6c([2H])c([2H])c([2H])c([2H])c6[2H])cc5)nc(-c5c([2H])c([2H])c([2H])c([2H])c5[2H])n4)c32)c([2H])c1[2H] ; [2H]c1cc([2H])c2oc3c([2H])c([2H])c(-c4ccc5oc6ccccc6c5c4)cc3c2c1-c1nc(-c2ccccc2)nc(-c2c([2H])c([2H])c3c(c2[2H])c([2H])c([2H])c2c([2H])c([2H])c([2H])c([2H])c23)n1 ; [2H]c1c([2H])c([2H])c(-c2nc(-c3ccc(-c4c([2H])c([2H])c([2H])c5c([2H])c([2H])c([2H])c([2H])c45)cc3)nc(-c3cccc4oc5ccc(-c6cccc7ccccc67)cc5c34)n2)c([2H])c1[2H]]

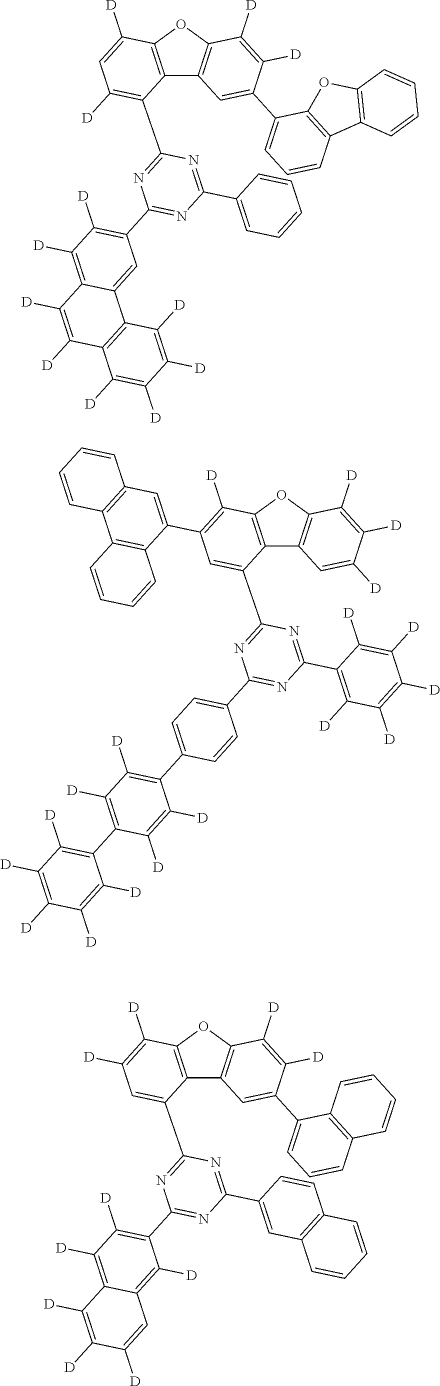

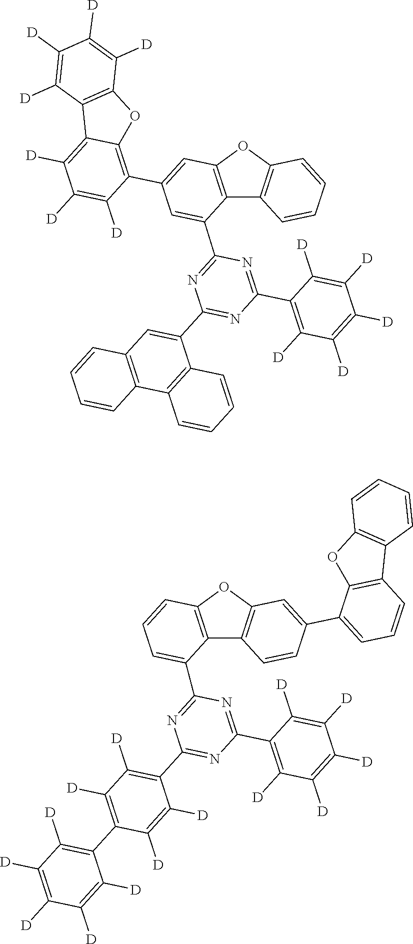 [SMILES: [2H]c1c([2H])c([2H])c(-c2nc(-c3cc4ccccc4c4ccccc34)nc(-c3cc(-c4c([2H])c([2H])c([2H])c5c4oc4c([2H])c([2H])c([2H])c([2H])c45)cc4oc5ccccc5c34)n2)c([2H])c1[2H] ; [2H]c1c([2H])c([2H])c(-c2nc(-c3c([2H])c([2H])c(-c4c([2H])c([2H])c([2H])c([2H])c4[2H])c([2H])c3[2H])nc(-c3cccc4oc5cc(-c6cccc7c6oc6ccccc67)ccc5c34)n2)c([2H])c1[2H]]

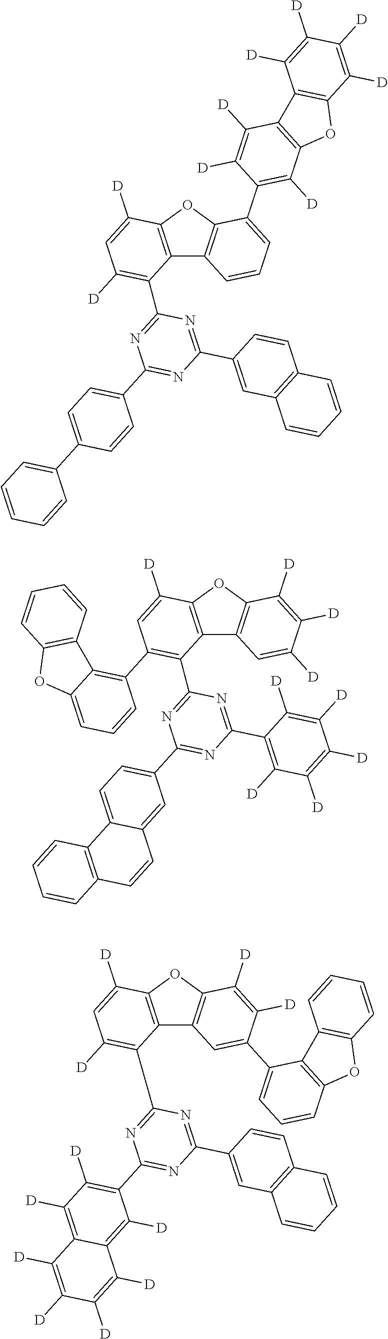 [SMILES: [2H]c1cc([2H])c2oc3c(-c4c([2H])c([2H])c5c(oc6c([2H])c([2H])c([2H])c([2H])c65)c4[2H])cccc3c2c1-c1nc(-c2ccc(-c3ccccc3)cc2)nc(-c2ccc3ccccc3c2)n1 ; [2H]c1cc2c(oc3c([2H])cc(-c4cccc5oc6ccccc6c45)c(-c4nc(-c5ccc6c(ccc7ccccc76)c5)nc(-c5c([2H])c([2H])c([2H])c([2H])c5[2H])n4)c32)c([2H])c1[2H] ; [2H]c1cc([2H])c2oc3c([2H])c([2H])c(-c4cccc5oc6ccccc6c45)cc3c2c1-c1nc(-c2ccc3ccccc3c2)nc(-c2c([2H])c([2H])c3c([2H])c([2H])c([2H])c([2H])c3c2[2H])n1]

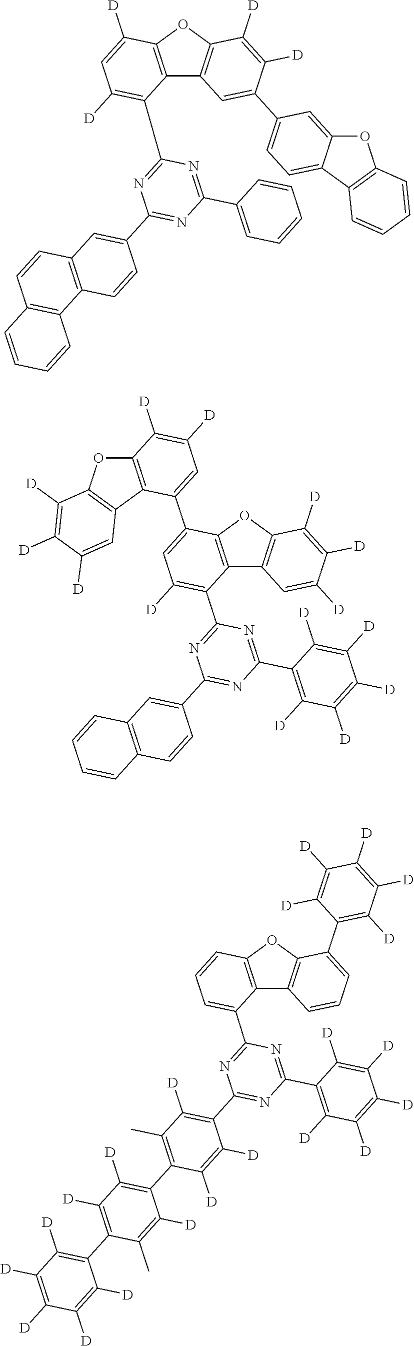 [SMILES: [2H]c1cc([2H])c2oc3c([2H])c([2H])c(-c4ccc5c(c4)oc4ccccc45)cc3c2c1-c1nc(-c2ccccc2)nc(-c2ccc3c(ccc4ccccc43)c2)n1 ; [2H]c1cc2c(oc3c([2H])c([2H])cc(-c4cc([2H])c(-c5nc(-c6ccc7ccccc7c6)nc(-c6c([2H])c([2H])c([2H])c([2H])c6[2H])n5)c5c4oc4c([2H])c([2H])c([2H])cc45)c32)c([2H])c1[2H] ; [2H]c1c([2H])c([2H])c(-c2nc(-c3c([2H])c([2H])c(-c4c([2H])c([2H])c(-c5c([2H])c([2H])c([2H])c([2H])c5[2H])c(C)c4[2H])c(C)c3[2H])nc(-c3cccc4oc5c(-c6c([2H])c([2H])c([2H])c([2H])c6[2H])cccc5c34)n2)c([2H])c1[2H]]

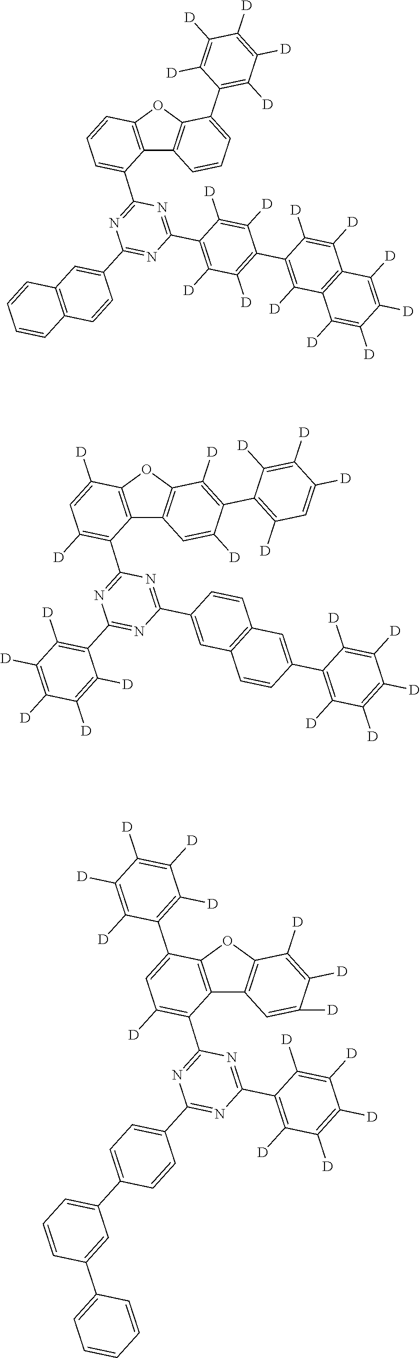

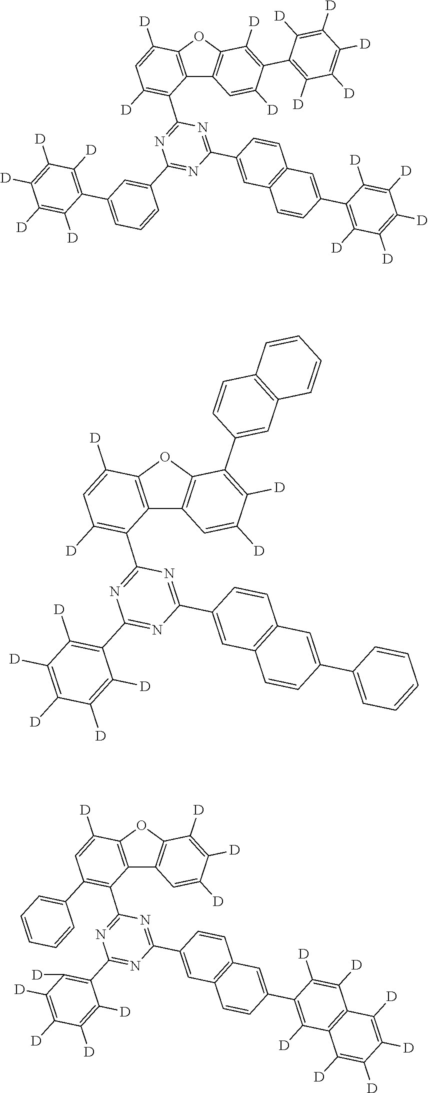

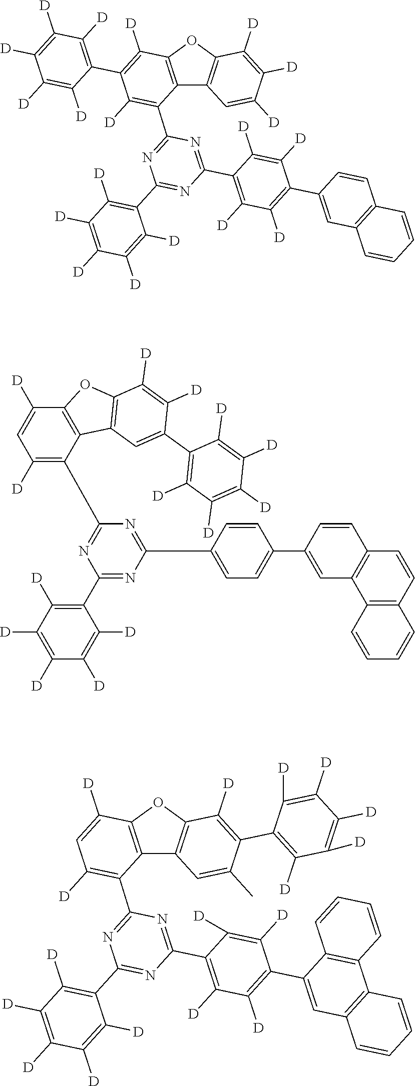

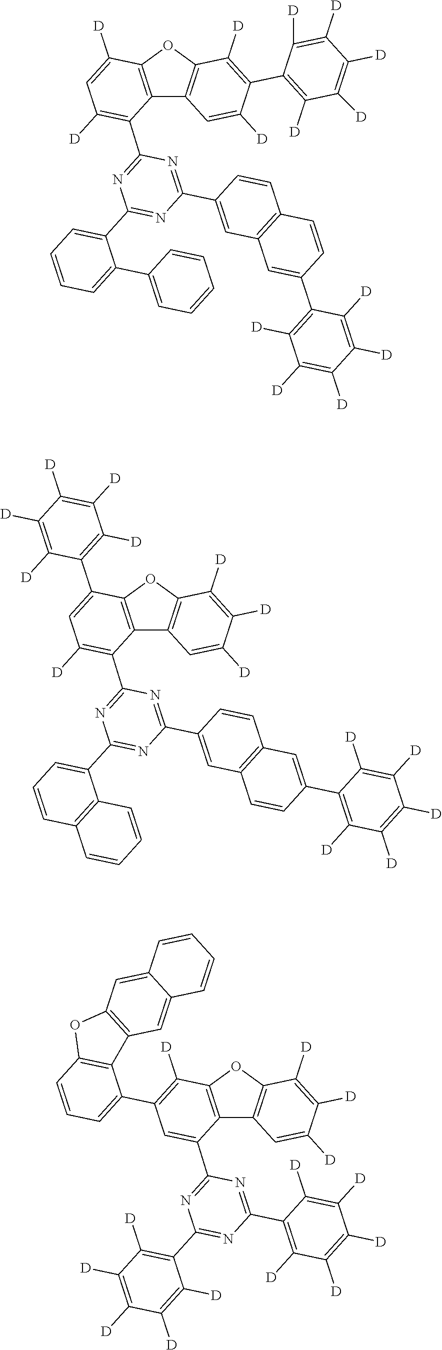 [SMILES: [2H]c1cc2c(oc3c([2H])cc([2H])c(-c4nc(-c5ccc6ccc(-c7c([2H])c([2H])c([2H])c([2H])c7[2H])cc6c5)nc(-c5ccccc5-c5ccccc5)n4)c32)c([2H])c1-c1c([2H])c([2H])c([2H])c([2H])c1[2H] ; [2H]c1cc2c(oc3c(-c4c([2H])c([2H])c([2H])c([2H])c4[2H])cc([2H])c(-c4nc(-c5ccc6cc(-c7c([2H])c([2H])c([2H])c([2H])c7[2H])ccc6c5)nc(-c5cccc6ccccc56)n4)c32)c([2H])c1[2H] ; [2H]c1cc2c(oc3c([2H])c(-c4cccc5oc6cc7ccccc7cc6c45)cc(-c4nc(-c5c([2H])c([2H])c([2H])c([2H])c5[2H])nc(-c5c([2H])c([2H])c([2H])c([2H])c5[2H])n4)c32)c([2H])c1[2H]]

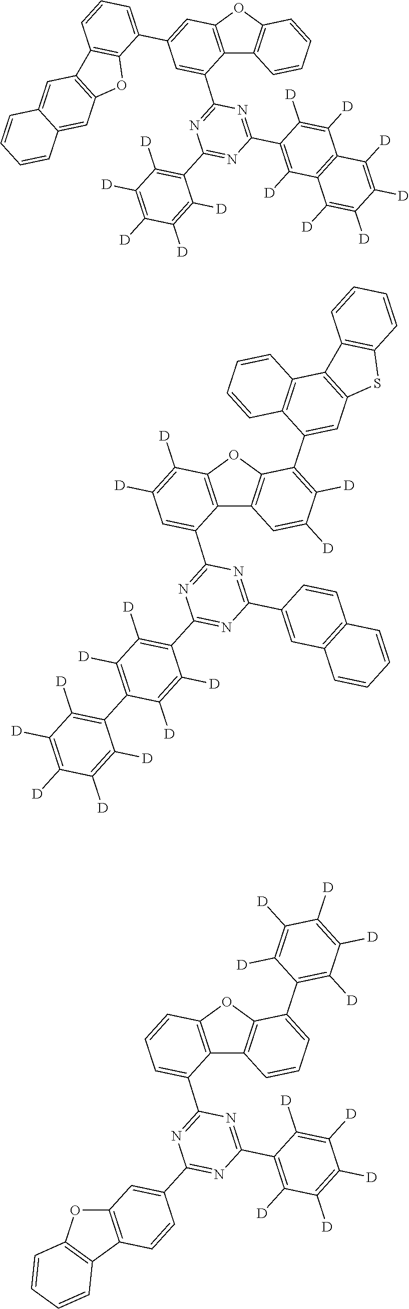 [SMILES: [2H]c1c([2H])c([2H])c(-c2nc(-c3c([2H])c([2H])c4c([2H])c([2H])c([2H])c([2H])c4c3[2H])nc(-c3cc(-c4cccc5c4oc4cc6ccccc6cc45)cc4oc5ccccc5c34)n2)c([2H])c1[2H] ; [2H]c1cc2c(oc3c([2H])c([2H])cc(-c4nc(-c5ccc6ccccc6c5)nc(-c5c([2H])c([2H])c(-c6c([2H])c([2H])c([2H])c([2H])c6[2H])c([2H])c5[2H])n4)c32)c(-c2cc3sc4ccccc4c3c3ccccc23)c1[2H] ; [2H]c1c([2H])c([2H])c(-c2nc(-c3ccc4c(c3)oc3ccccc34)nc(-c3cccc4oc5c(-c6c([2H])c([2H])c([2H])c([2H])c6[2H])cccc5c34)n2)c([2H])c1[2H]]

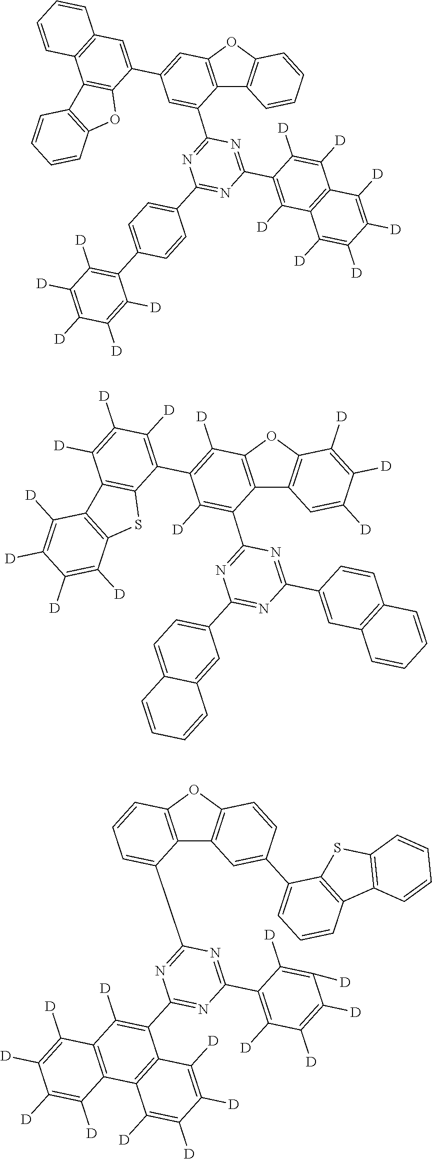

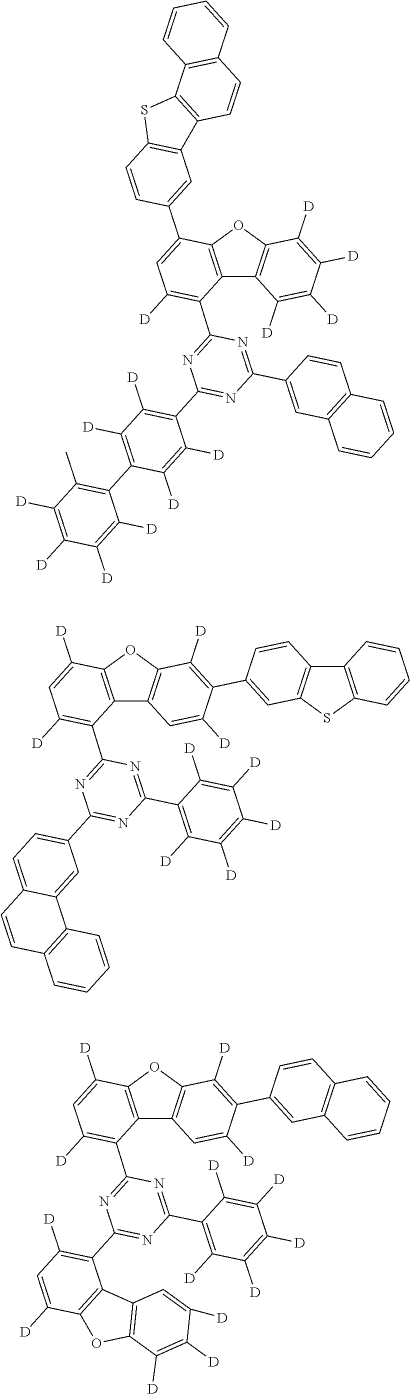 [SMILES: [2H]c1cc(-c2ccc3sc4c5ccccc5ccc4c3c2)c2oc3c([2H])c([2H])c([2H])c([2H])c3c2c1-c1nc(-c2ccc3ccccc3c2)nc(-c2c([2H])c([2H])c(-c3c([2H])c([2H])c([2H])c([2H])c3C)c([2H])c2[2H])n1 ; [2H]c1cc2c(oc3c([2H])cc([2H])c(-c4nc(-c5ccc6ccc7ccccc7c6c5)nc(-c5c([2H])c([2H])c([2H])c([2H])c5[2H])n4)c32)c([2H])c1-c1ccc2c(c1)sc1ccccc12 ; [2H]c1cc2c(oc3c([2H])cc([2H])c(-c4nc(-c5c([2H])c([2H])c([2H])c([2H])c5[2H])nc(-c5c([2H])cc([2H])c6oc7c([2H])c(-c8ccc9ccccc9c8)c([2H])cc7c56)n4)c32)c([2H])c1[2H]]

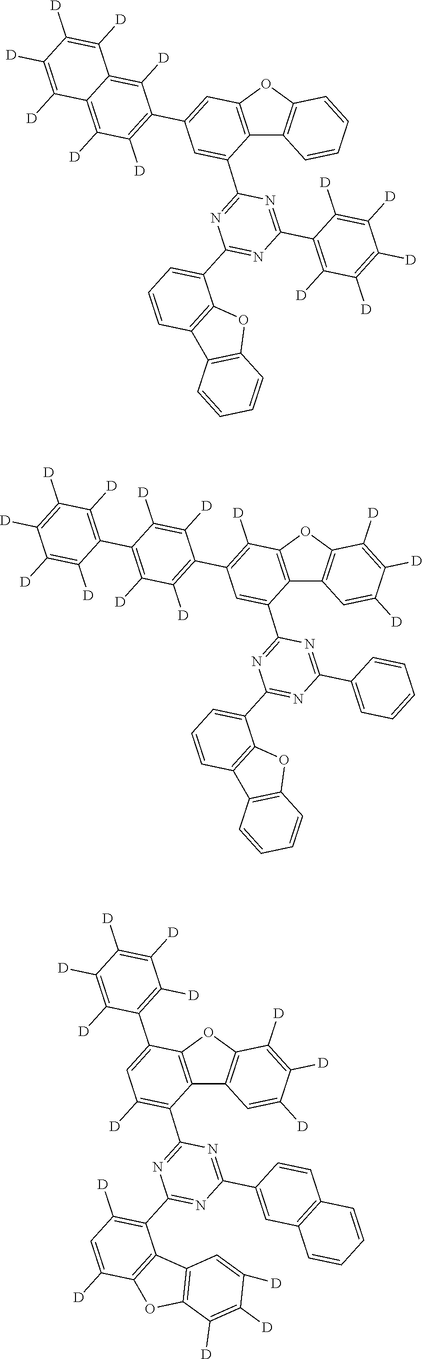 [SMILES: [2H]c1c([2H])c([2H])c(-c2nc(-c3cccc4c3oc3ccccc34)nc(-c3cc(-c4c([2H])c([2H])c5c([2H])c([2H])c([2H])c([2H])c5c4[2H])cc4oc5ccccc5c34)n2)c([2H])c1[2H] ; [2H]c1cc2c(oc3c([2H])c(-c4c([2H])c([2H])c(-c5c([2H])c([2H])c([2H])c([2H])c5[2H])c([2H])c4[2H])cc(-c4nc(-c5ccccc5)nc(-c5cccc6c5oc5ccccc56)n4)c32)c([2H])c1[2H] ; [2H]c1cc2c(oc3c([2H])cc([2H])c(-c4nc(-c5ccc6ccccc6c5)nc(-c5c([2H])cc(-c6c([2H])c([2H])c([2H])c([2H])c6[2H])c6oc7c([2H])c([2H])c([2H])cc7c56)n4)c32)c([2H])c1[2H]]

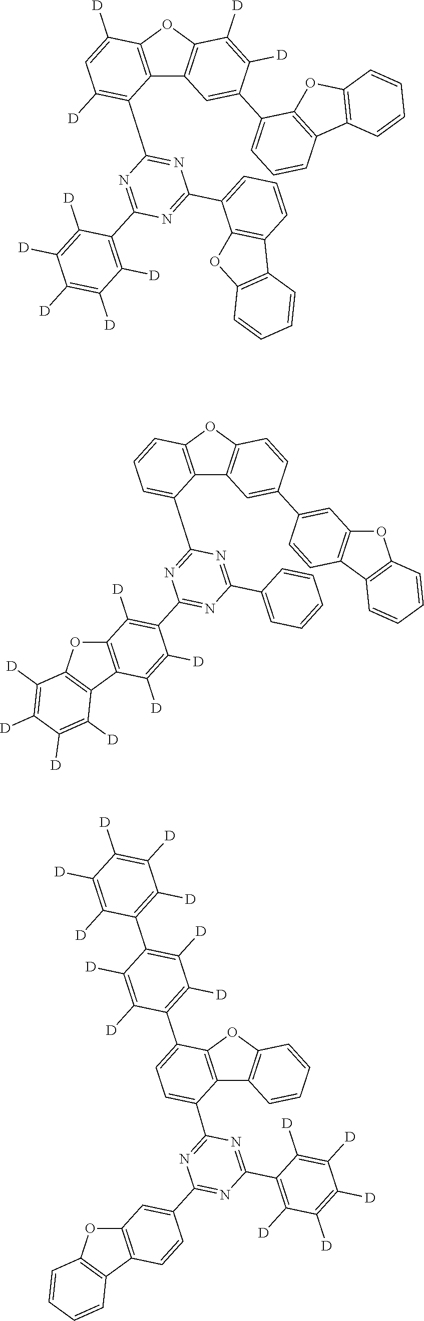 [SMILES: [2H]c1cc([2H])c2oc3c([2H])c([2H])c(-c4cccc5c4oc4ccccc45)cc3c2c1-c1nc(-c2c([2H])c([2H])c([2H])c([2H])c2[2H])nc(-c2cccc3c2oc2ccccc23)n1 ; [2H]c1c([2H])c([2H])c2c(oc3c([2H])c(-c4nc(-c5ccccc5)nc(-c5cccc6oc7ccc(-c8ccc9c(c8)oc8ccccc89)cc7c56)n4)c([2H])c([2H])c32)c1[2H] ; [2H]c1c([2H])c([2H])c(-c2nc(-c3ccc4c(c3)oc3ccccc34)nc(-c3ccc(-c4c([2H])c([2H])c(-c5c([2H])c([2H])c([2H])c([2H])c5[2H])c([2H])c4[2H])c4oc5ccccc5c34)n2)c([2H])c1[2H]]

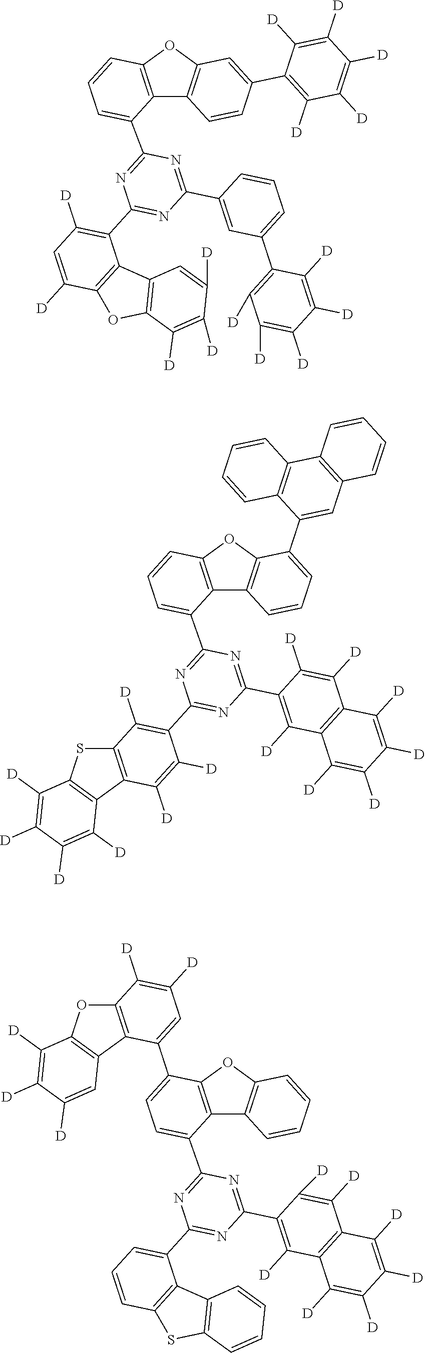

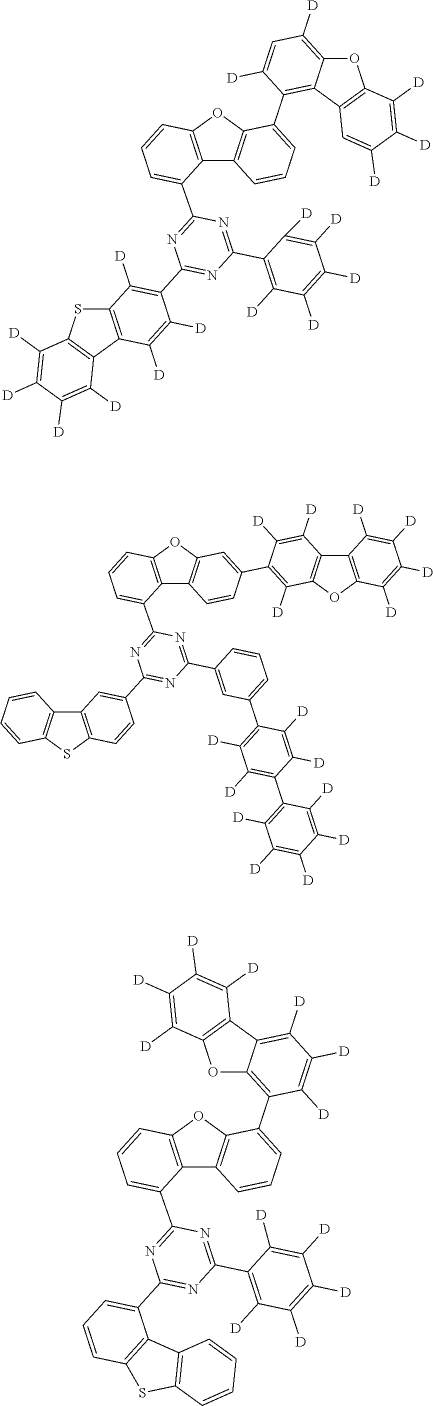

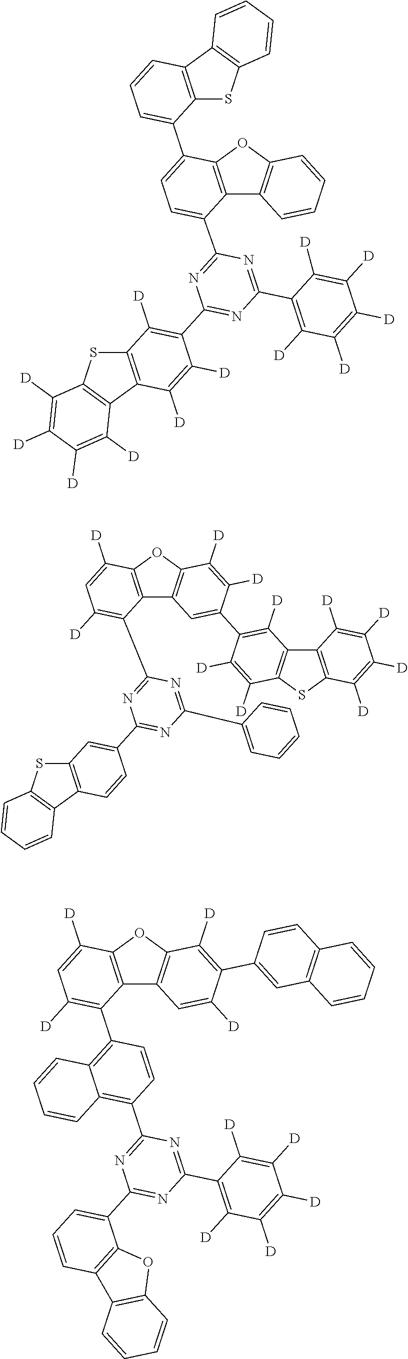 [SMILES: [2H]c1c([2H])c([2H])c(-c2nc(-c3c([2H])c([2H])c4c(sc5c([2H])c([2H])c([2H])c([2H])c54)c3[2H])nc(-c3ccc(-c4cccc5c4sc4ccccc45)c4oc5ccccc5c34)n2)c([2H])c1[2H] ; [2H]c1cc([2H])c2oc3c([2H])c([2H])c(-c4c([2H])c([2H])c5sc6c([2H])c([2H])c([2H])c([2H])c6c5c4[2H])cc3c2c1-c1nc(-c2ccccc2)nc(-c2ccc3c(c2)sc2ccccc23)n1 ; [2H]c1cc2c(oc3c([2H])cc([2H])c(-c4ccc(-c5nc(-c6c([2H])c([2H])c([2H])c([2H])c6[2H])nc(-c6cccc7c6oc6ccccc67)n5)c5ccccc45)c32)c([2H])c1-c1ccc2ccccc2c1]

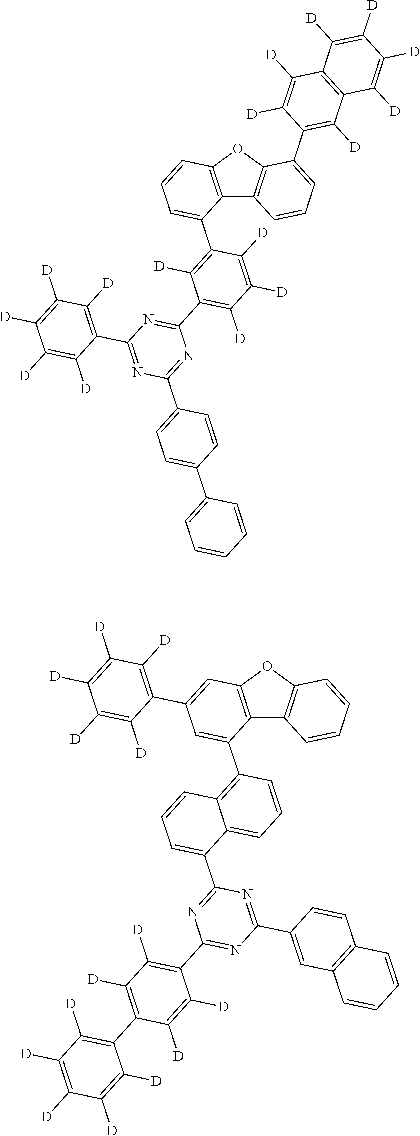 [SMILES: [2H]c1c([2H])c([2H])c(-c2nc(-c3ccc(-c4ccccc4)cc3)nc(-c3c([2H])c([2H])c([2H])c(-c4cccc5oc6c(-c7c([2H])c([2H])c8c([2H])c([2H])c([2H])c([2H])c8c7[2H])cccc6c45)c3[2H])n2)c([2H])c1[2H] ; [2H]c1c([2H])c([2H])c(-c2cc(-c3cccc4c(-c5nc(-c6ccc7ccccc7c6)nc(-c6c([2H])c([2H])c(-c7c([2H])c([2H])c([2H])c([2H])c7[2H])c([2H])c6[2H])n5)cccc34)c3c(c2)oc2ccccc23)c([2H])c1[2H]]

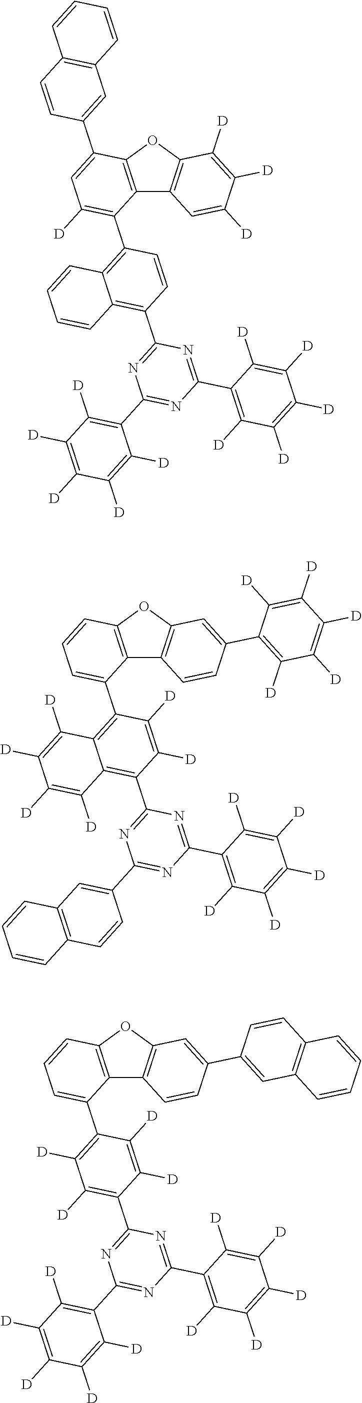

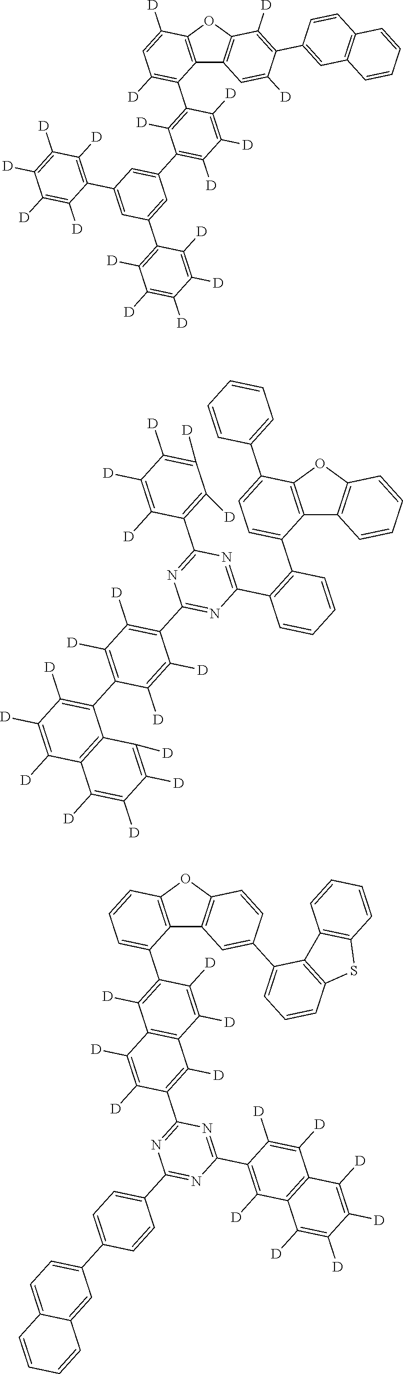

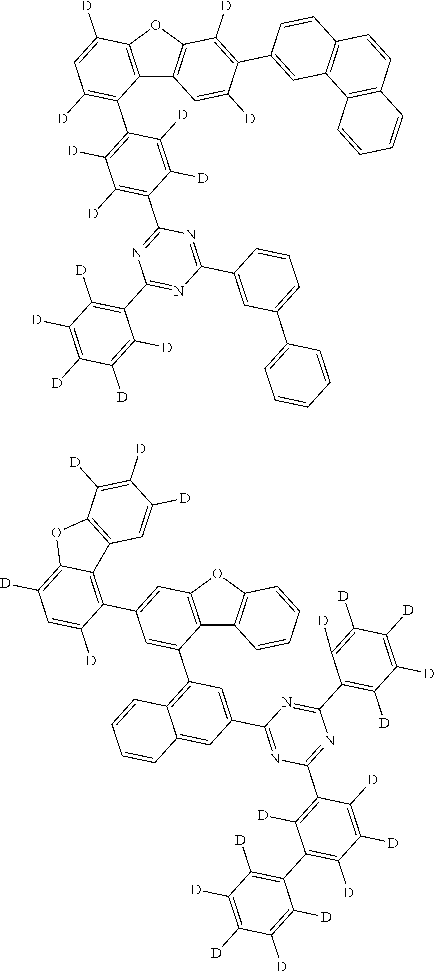 [SMILES: [2H]c1cc2c(oc3c([2H])cc([2H])c(-c4c([2H])c([2H])c(-c5nc(-c6cccc(-c7ccccc7)c6)nc(-c6c([2H])c([2H])c([2H])c([2H])c6[2H])n5)c([2H])c4[2H])c32)c([2H])c1-c1ccc2ccc3ccccc3c2c1 ; [2H]c1cc2c(oc3c([2H])cc([2H])c(-c4cc(-c5cc(-c6nc(-c7c([2H])c([2H])c([2H])c([2H])c7[2H])nc(-c7c([2H])c([2H])c([2H])c(-c8c([2H])c([2H])c([2H])c([2H])c8[2H])c7[2H])n6)cc6ccccc56)c5c(c4)oc4ccccc45)c32)c([2H])c1[2H]]

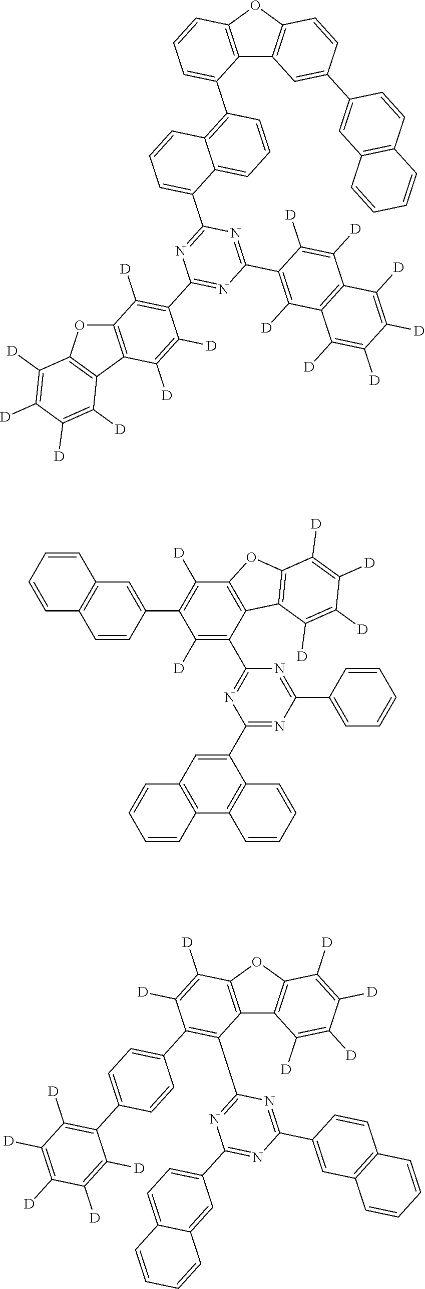 [SMILES: [2H]c1c([2H])c([2H])c2c(oc3c([2H])c(-c4nc(-c5c([2H])c([2H])c6c([2H])c([2H])c([2H])c([2H])c6c5[2H])nc(-c5cccc6c(-c7cccc8oc9ccc(-c%10ccc%11ccccc%11c%10)cc9c78)cccc56)n4)c([2H])c([2H])c32)c1[2H] ; [2H]c1c([2H])c([2H])c2c(oc3c([2H])c(-c4ccc5ccccc5c4)c([2H])c(-c4nc(-c5ccccc5)nc(-c5cc6ccccc6c6ccccc56)n4)c32)c1[2H] ; [2H]c1c([2H])c([2H])c(-c2ccc(-c3c([2H])c([2H])c4oc5c([2H])c([2H])c([2H])c([2H])c5c4c3-c3nc(-c4ccc5ccccc5c4)nc(-c4ccc5ccccc5c4)n3)cc2)c([2H])c1[2H]]

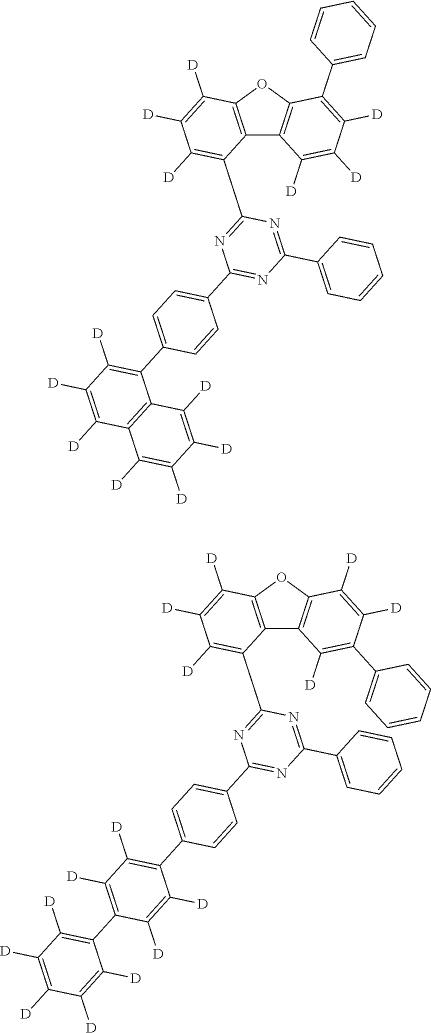 [SMILES: [2H]c1c([2H])c(-c2nc(-c3ccccc3)nc(-c3ccc(-c4c([2H])c([2H])c([2H])c5c([2H])c([2H])c([2H])c([2H])c45)cc3)n2)c2c(oc3c(-c4ccccc4)c([2H])c([2H])c([2H])c32)c1[2H] ; [2H]c1c([2H])c([2H])c(-c2c([2H])c([2H])c(-c3ccc(-c4nc(-c5ccccc5)nc(-c5c([2H])c([2H])c([2H])c6oc7c([2H])c([2H])c(-c8ccccc8)c([2H])c7c56)n4)cc3)c([2H])c2[2H])c([2H])c1[2H]]

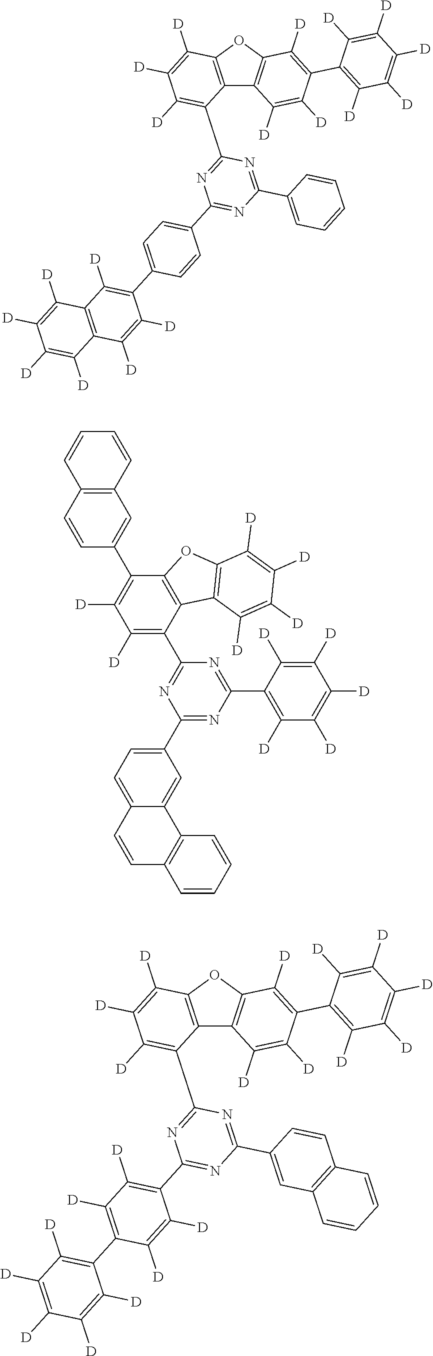

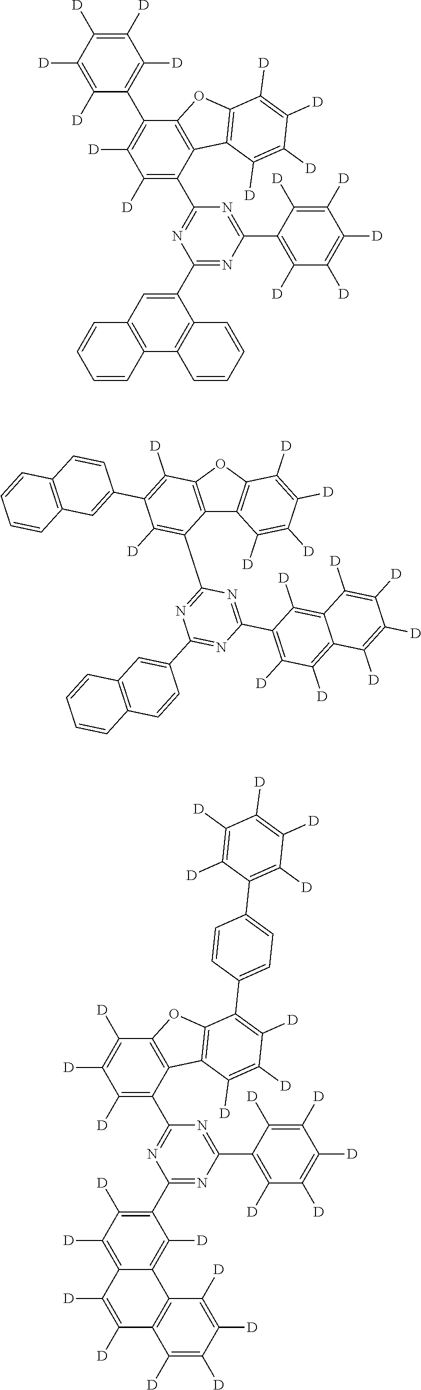

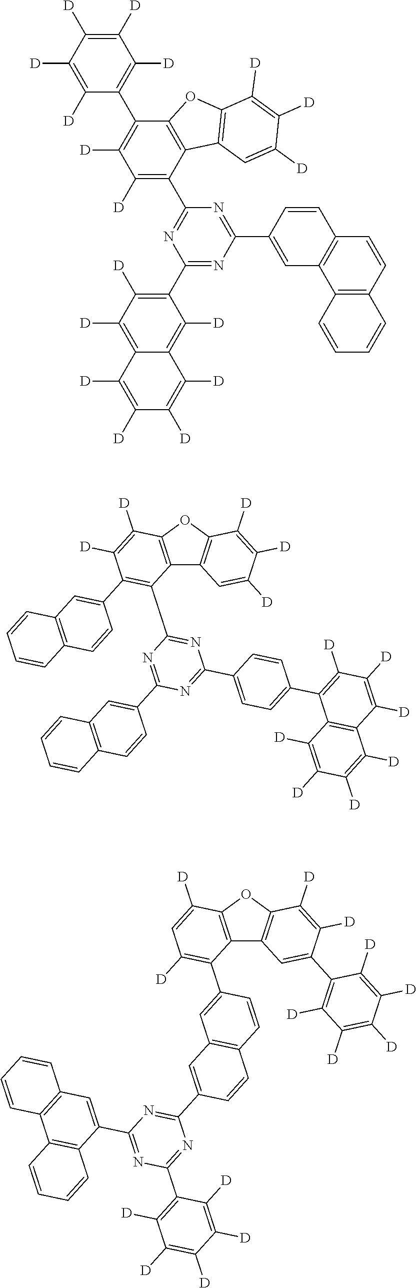

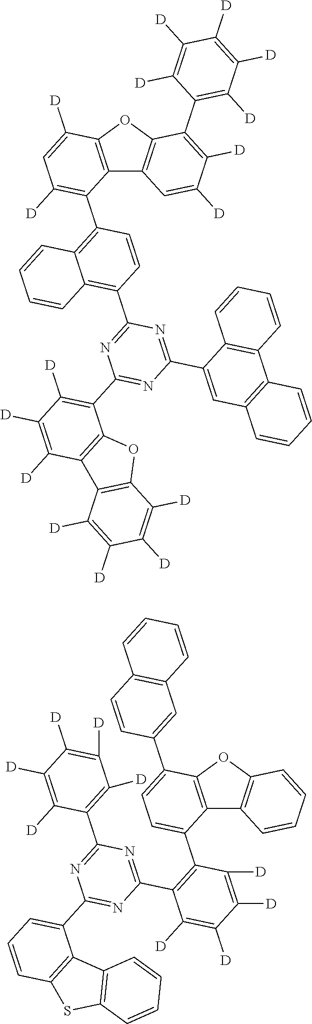 [SMILES: [2H]c1cc2c(oc3c([2H])cc([2H])c(-c4ccc(-c5nc(-c6cc7ccccc7c7ccccc67)nc(-c6c([2H])c([2H])c([2H])c7c6oc6c([2H])c([2H])c([2H])c([2H])c67)n5)c5ccccc45)c32)c(-c2c([2H])c([2H])c([2H])c([2H])c2[2H])c1[2H] ; [2H]c1c([2H])c([2H])c(-c2nc(-c3c([2H])c([2H])c([2H])c([2H])c3-c3ccc(-c4ccc5ccccc5c4)c4oc5ccccc5c34)nc(-c3cccc4sc5ccccc5c34)n2)c([2H])c1[2H]]

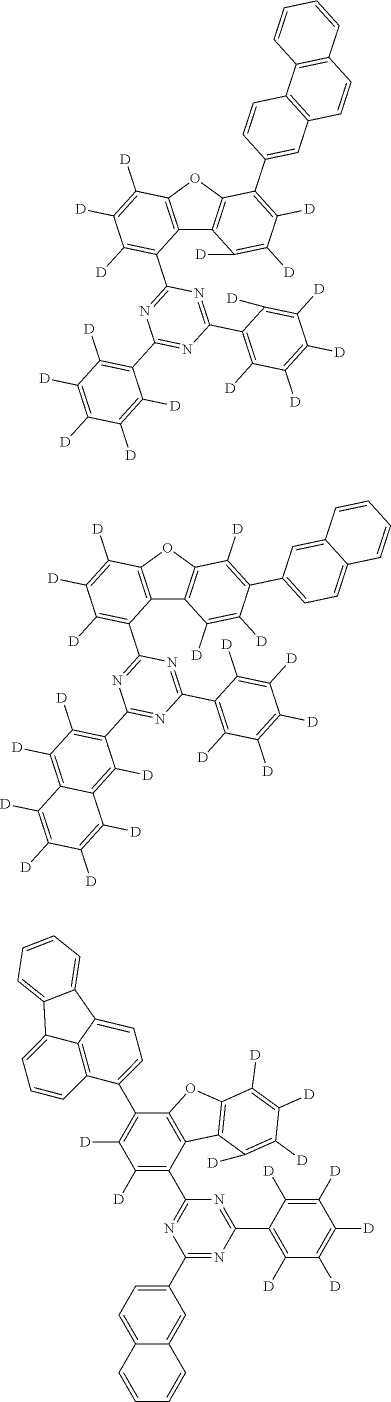

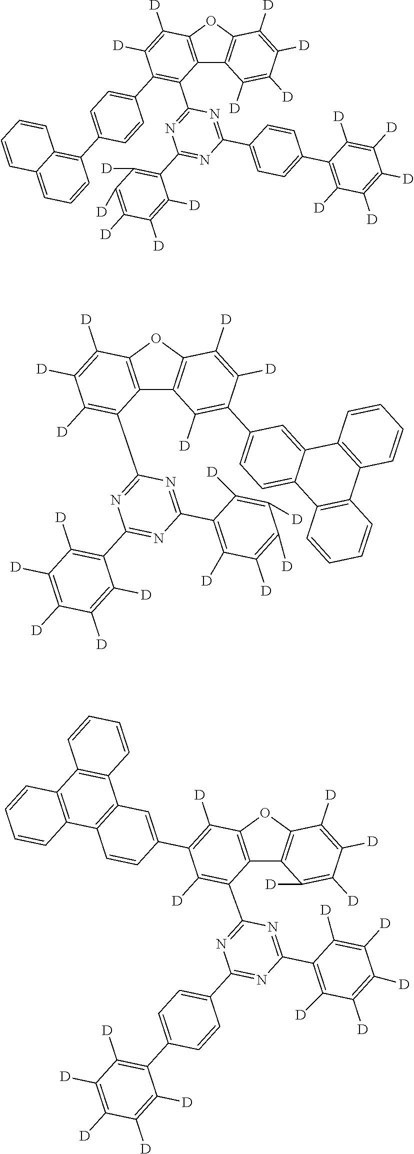

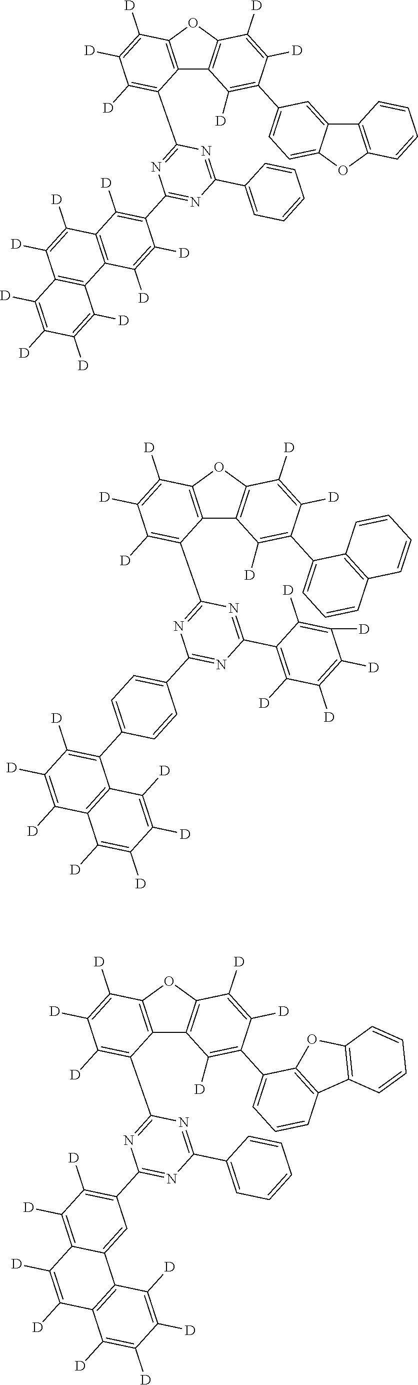

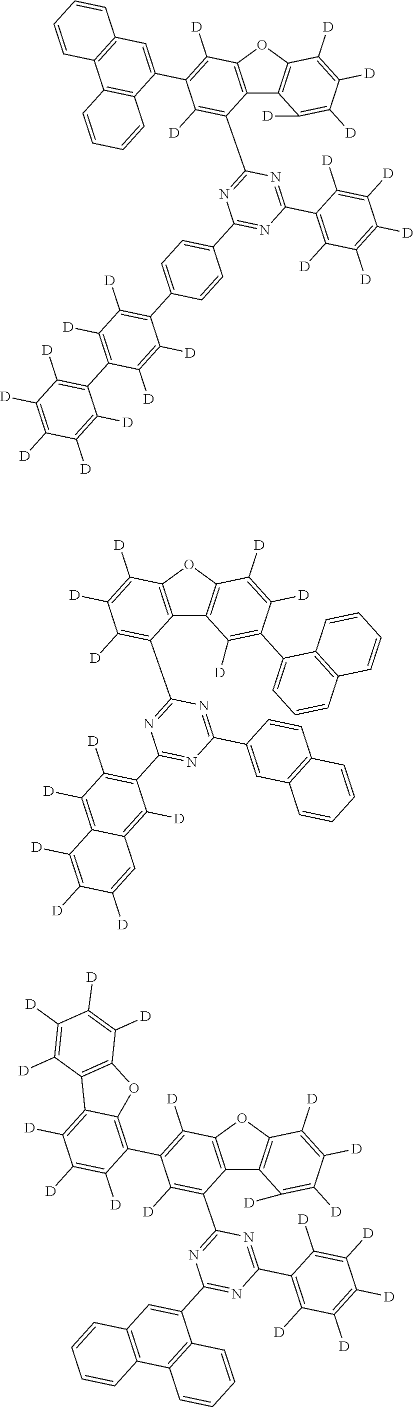

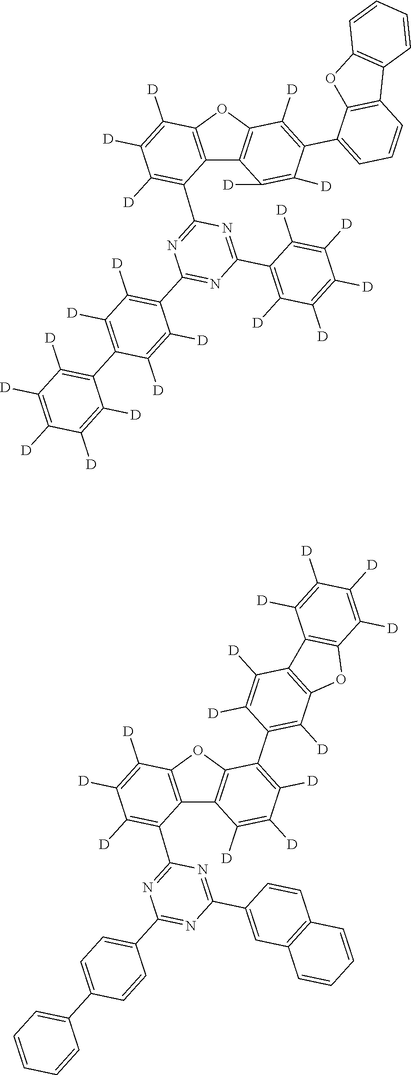 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c([2H])c4oc5c([2H])c(-c6cccc7c6oc6ccccc67)c([2H])c([2H])c5c34)n2)c([2H])c1[2H] ; [2H]c1c([2H])c([2H])c2c(oc3c([2H])c(-c4c([2H])c([2H])c([2H])c5c4oc4c([2H])c([2H])c([2H])c(-c6nc(-c7ccc(-c8ccccc8)cc7)nc(-c7ccc8ccccc8c7)n6)c45)c([2H])c([2H])c32)c1[2H]]

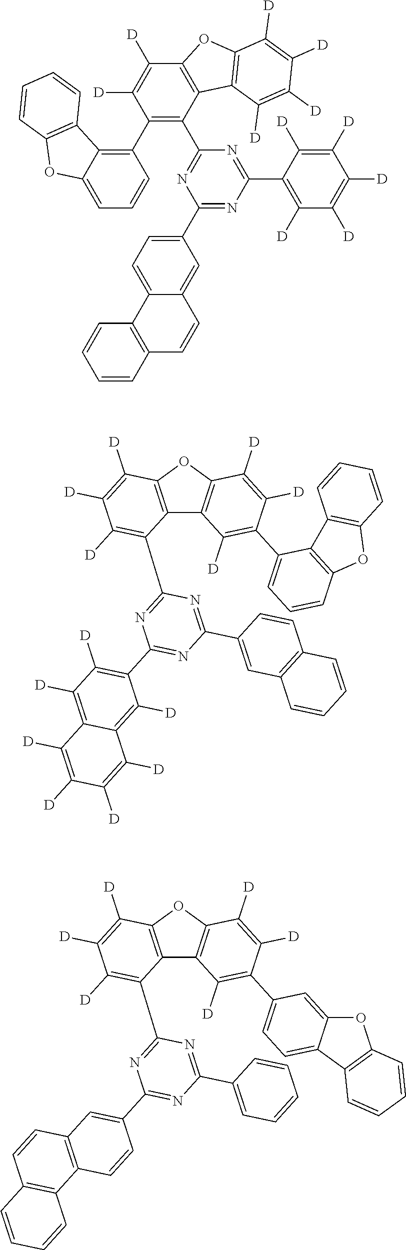 [SMILES: [2H]c1c([2H])c([2H])c(-c2nc(-c3ccc4c(ccc5ccccc54)c3)nc(-c3c(-c4cccc5oc6ccccc6c45)c([2H])c([2H])c4oc5c([2H])c([2H])c([2H])c([2H])c5c34)n2)c([2H])c1[2H] ; [2H]c1c(-c2cccc3oc4ccccc4c23)c([2H])c2c(oc3c([2H])c([2H])c([2H])c(-c4nc(-c5ccc6ccccc6c5)nc(-c5c([2H])c([2H])c6c([2H])c([2H])c([2H])c([2H])c6c5[2H])n4)c32)c1[2H] ; [2H]c1c(-c2ccc3c(c2)oc2ccccc23)c([2H])c2c(oc3c([2H])c([2H])c([2H])c(-c4nc(-c5ccccc5)nc(-c5ccc6c(ccc7ccccc76)c5)n4)c32)c1[2H]]

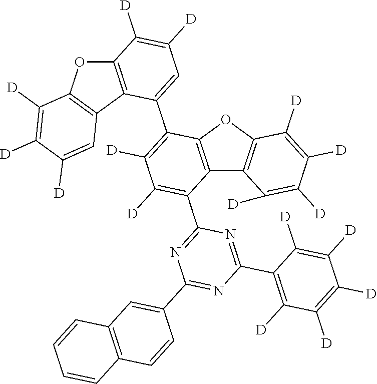 [SMILES: [2H]c1cc2c(oc3c([2H])c([2H])cc(-c4c([2H])c([2H])c(-c5nc(-c6ccc7ccccc7c6)nc(-c6c([2H])c([2H])c([2H])c([2H])c6[2H])n5)c5c4oc4c([2H])c([2H])c([2H])c([2H])c45)c32)c([2H])c1[2H]]

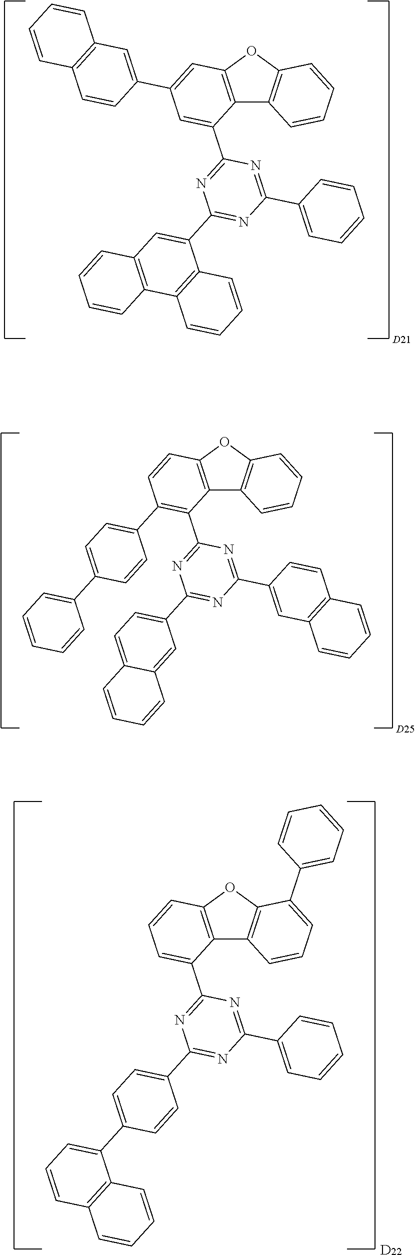

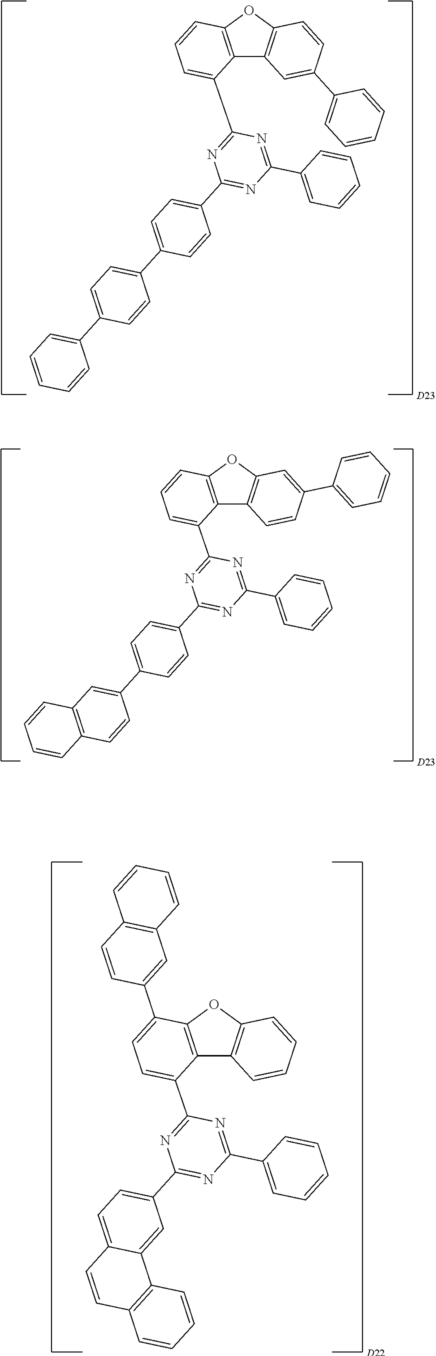

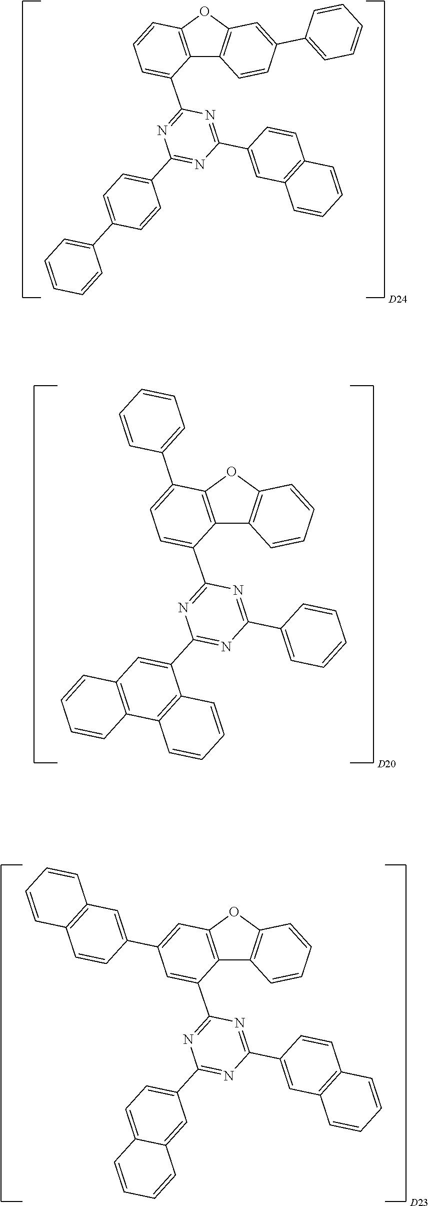

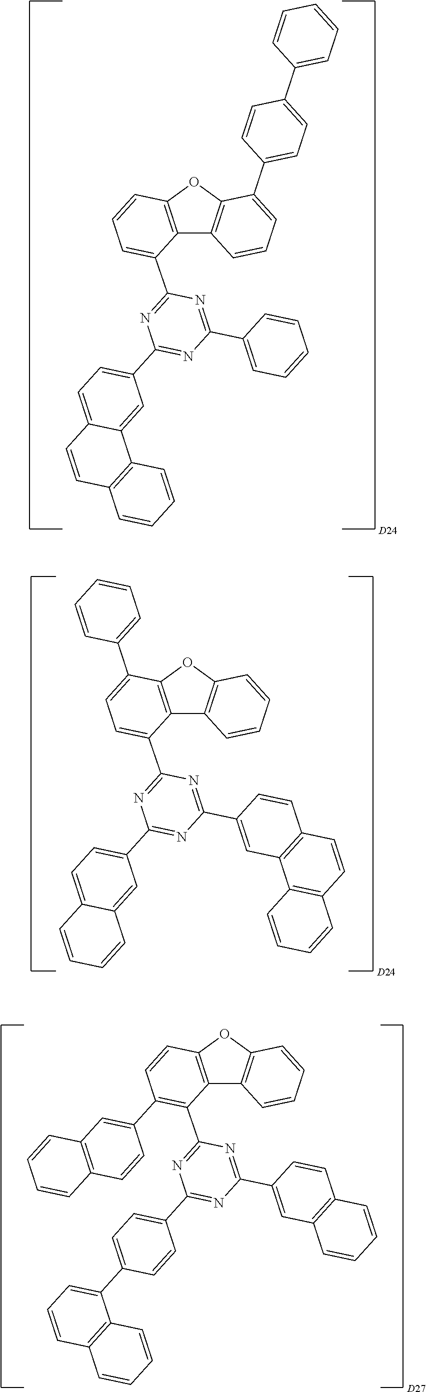

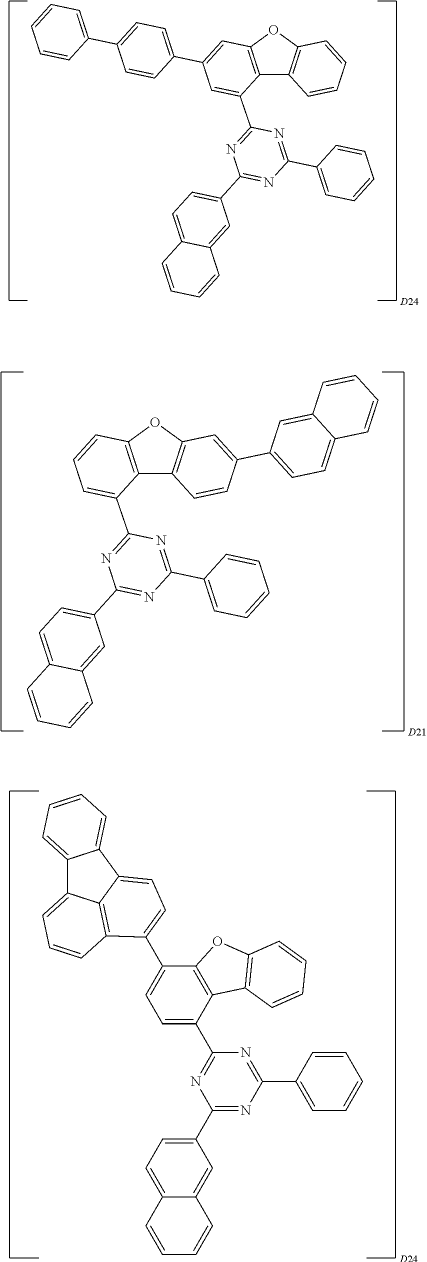

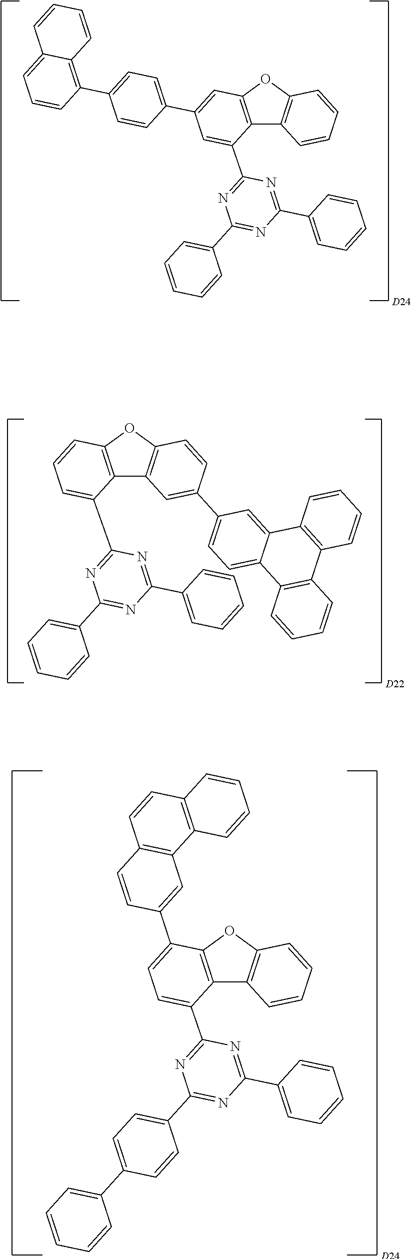

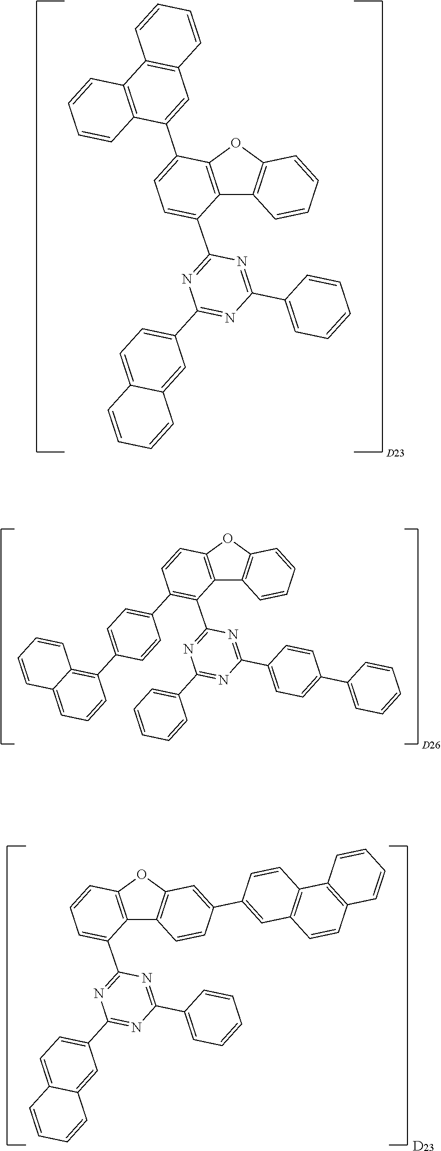

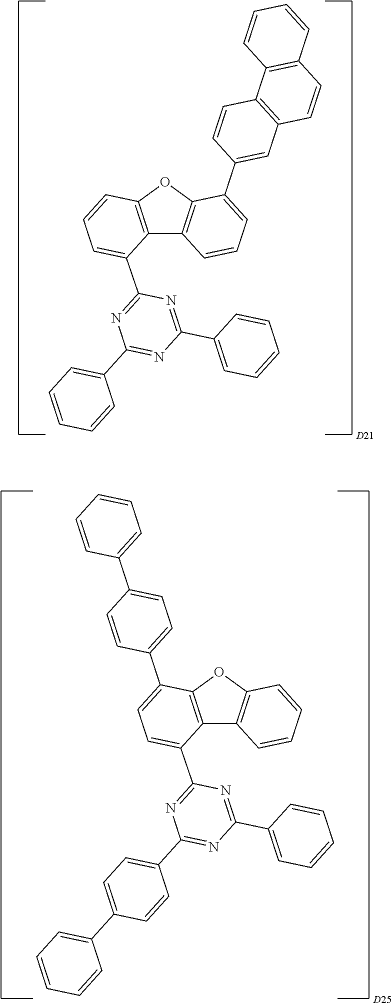

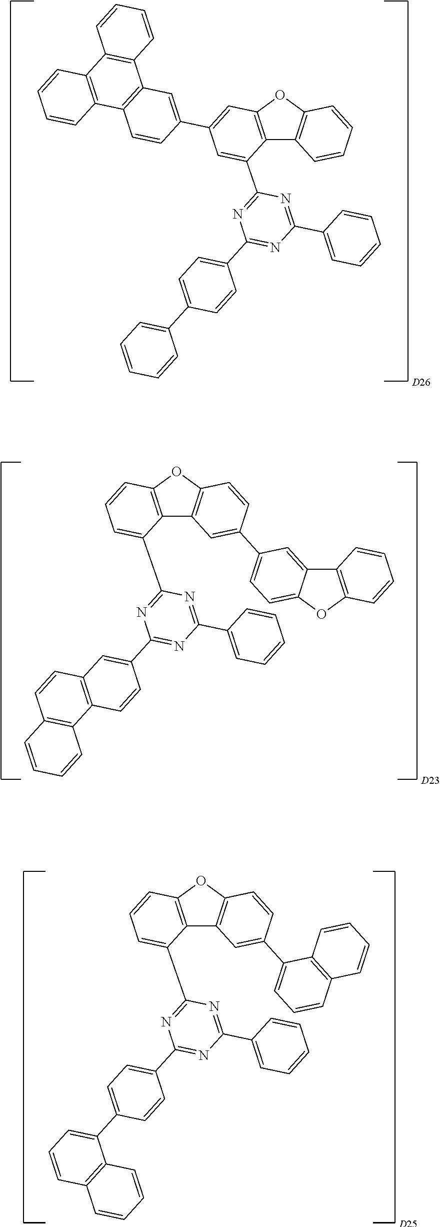

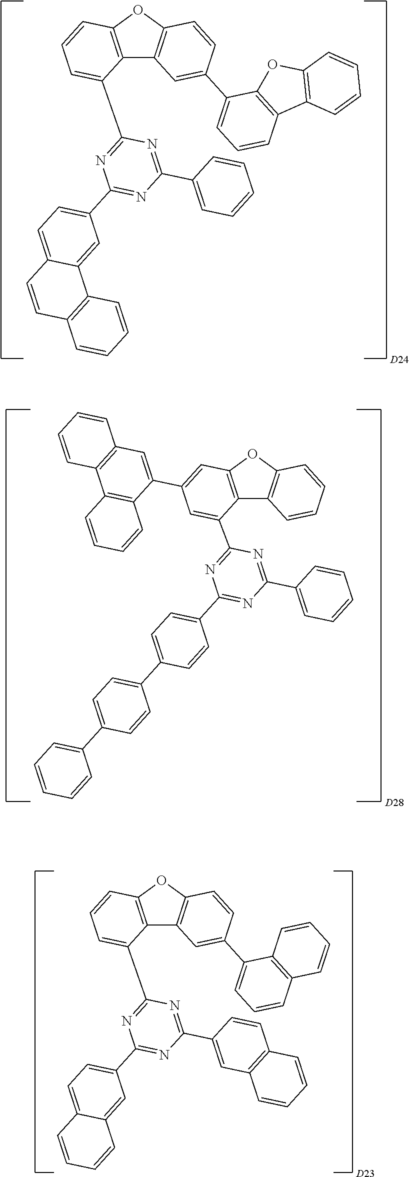

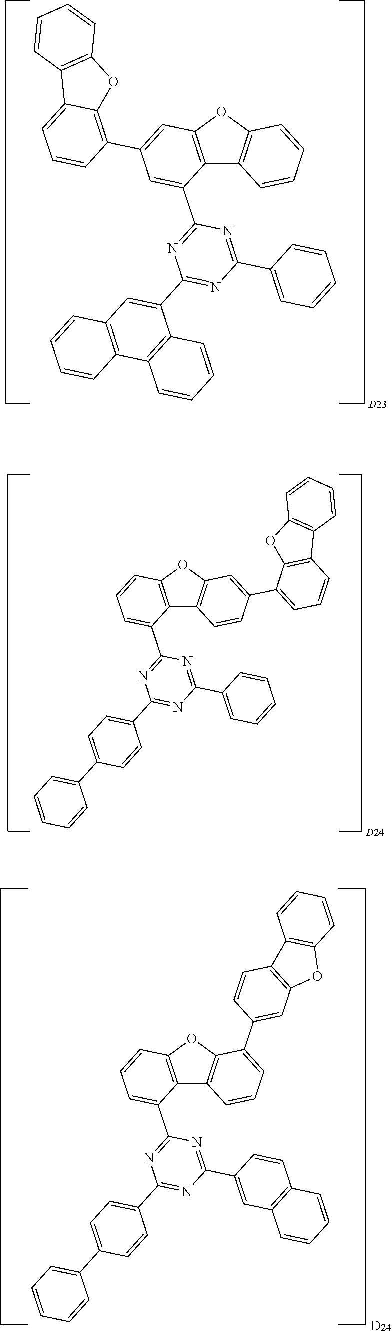

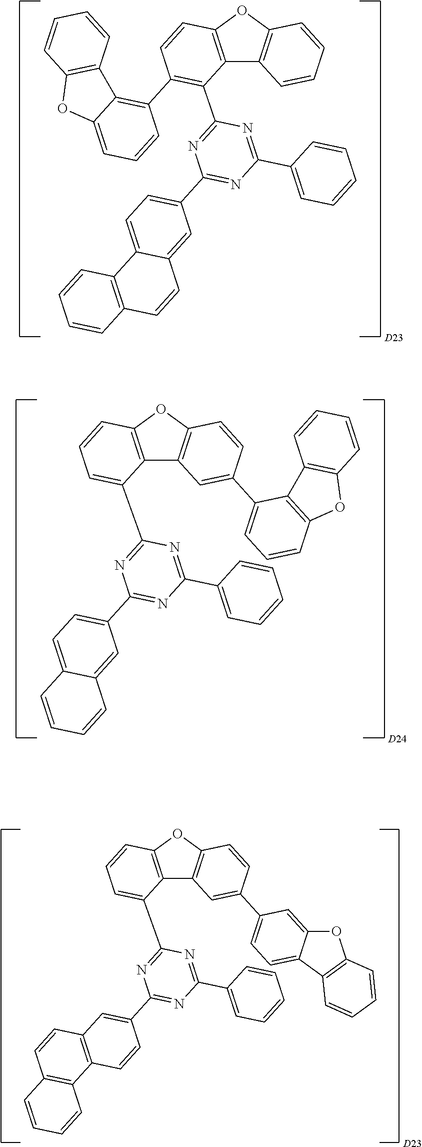

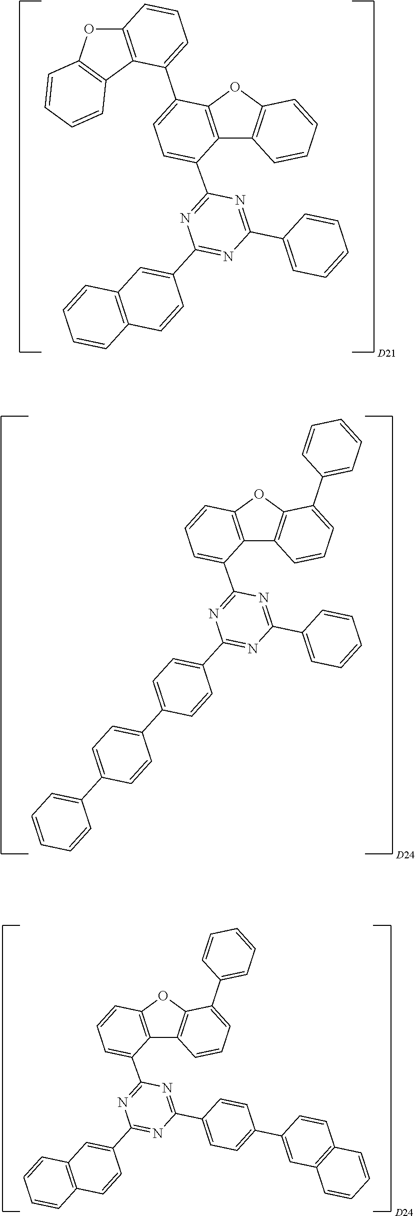

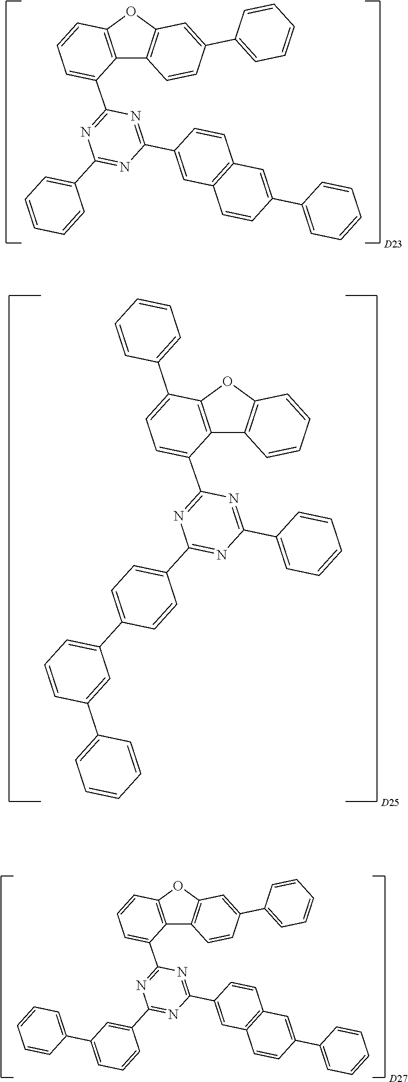

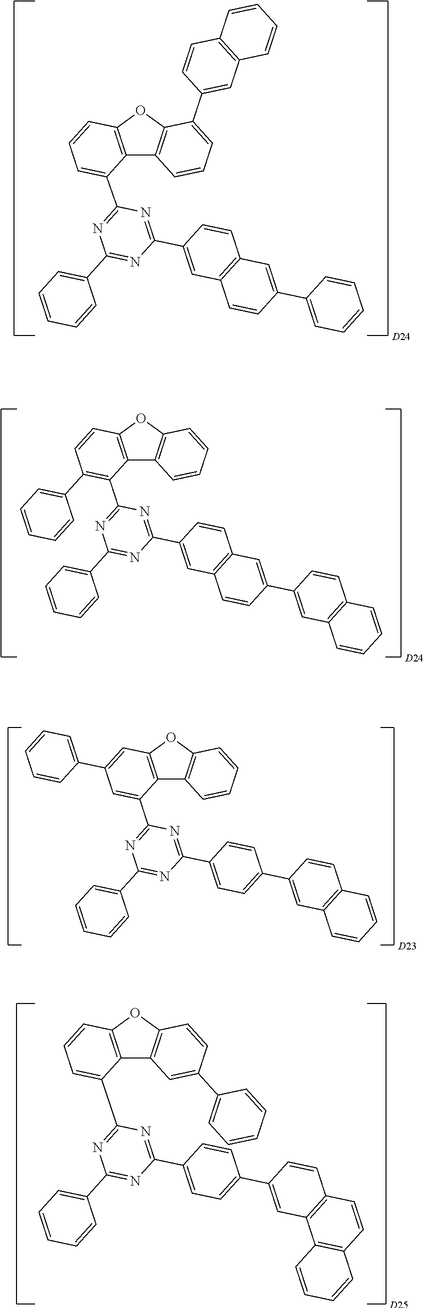

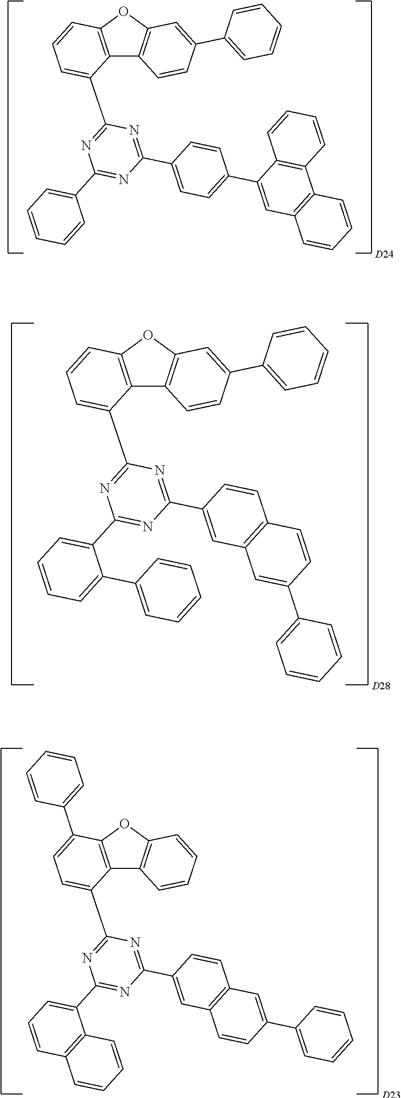

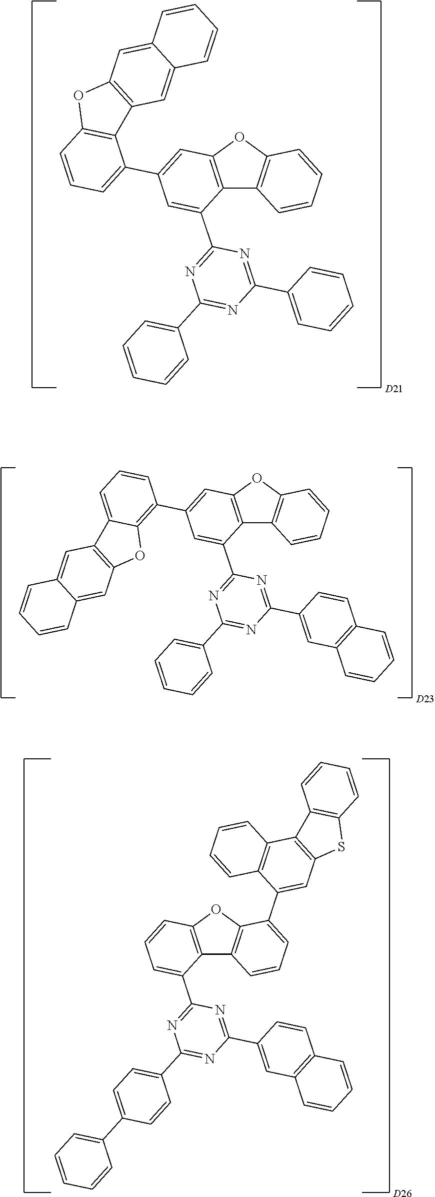

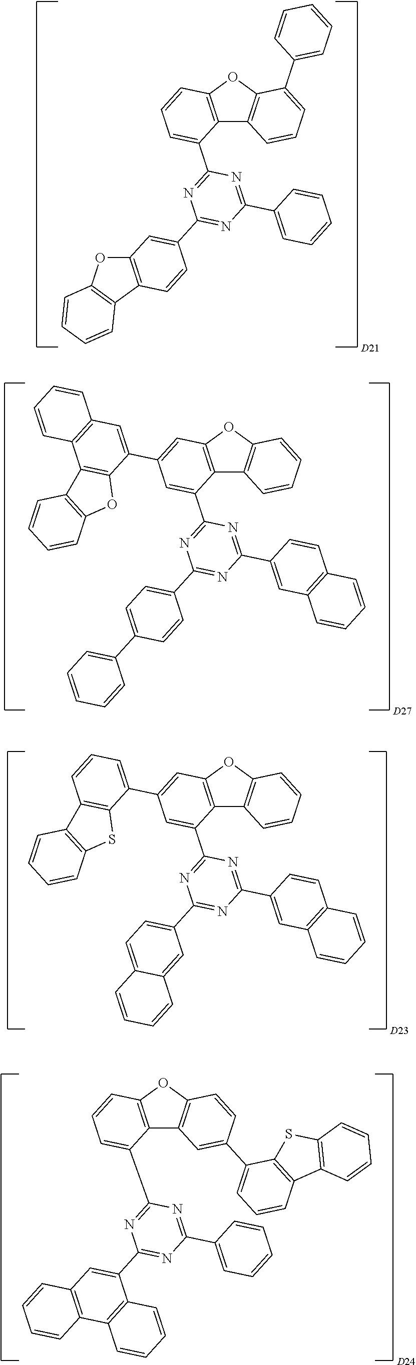

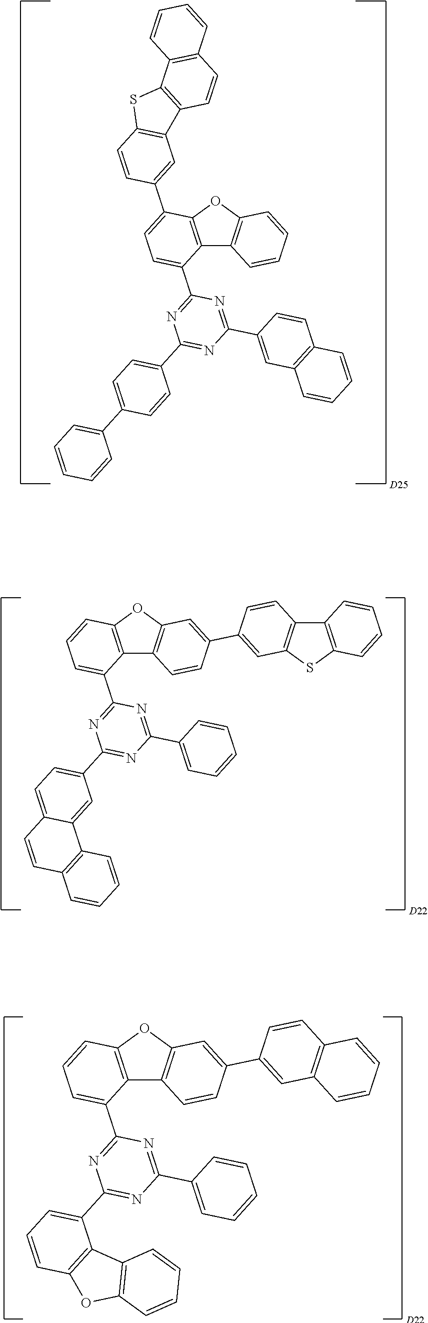

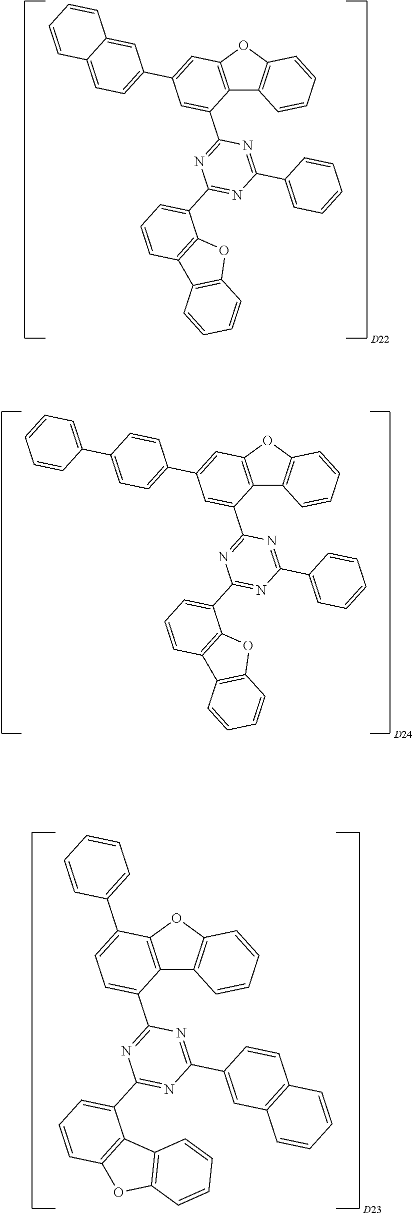

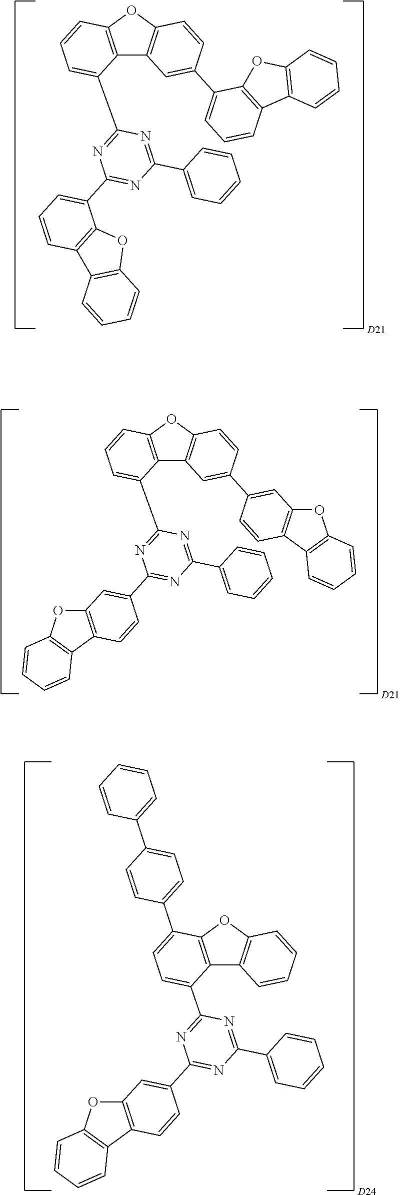

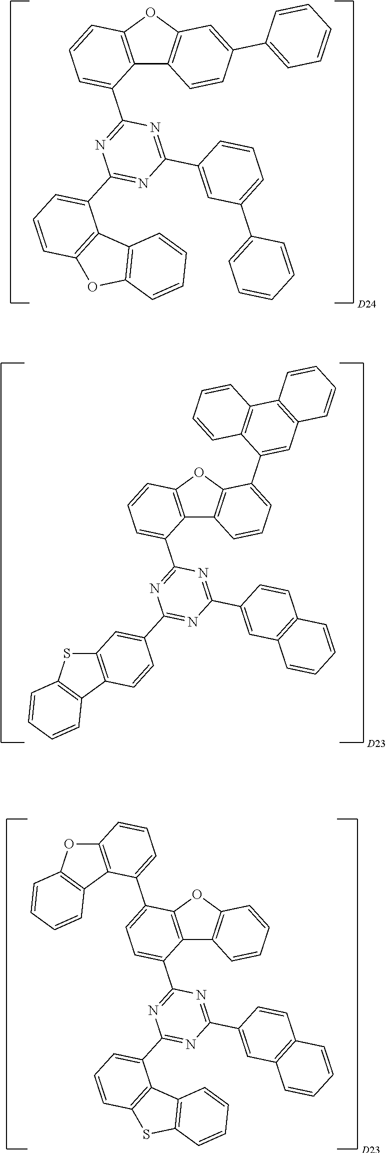

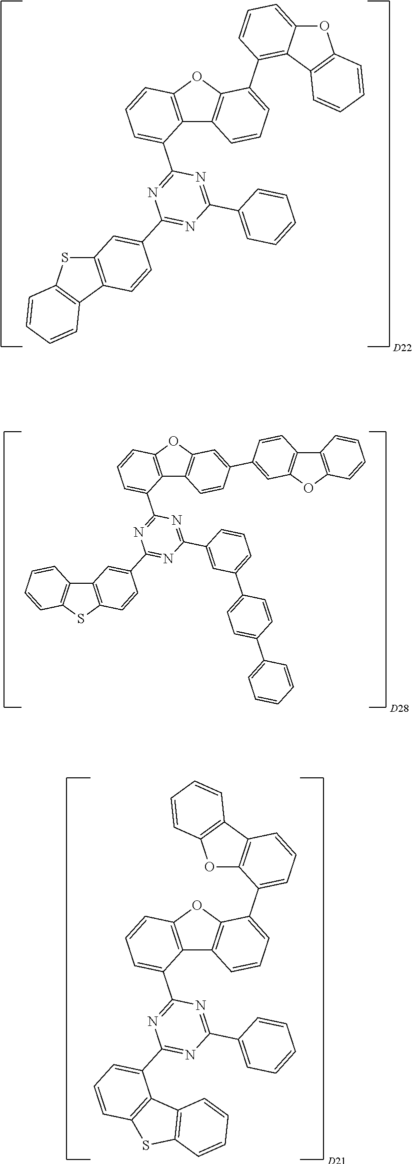

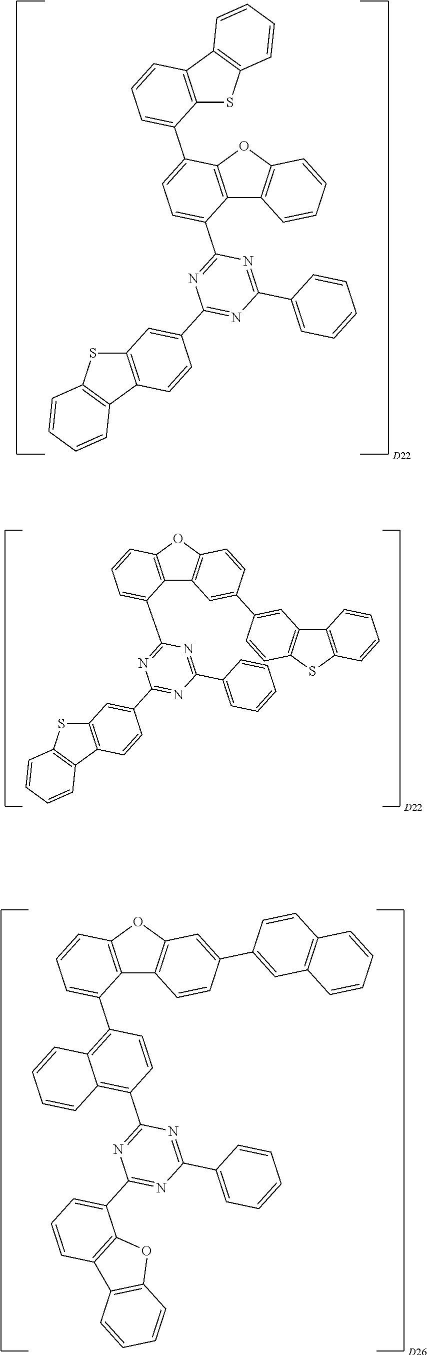

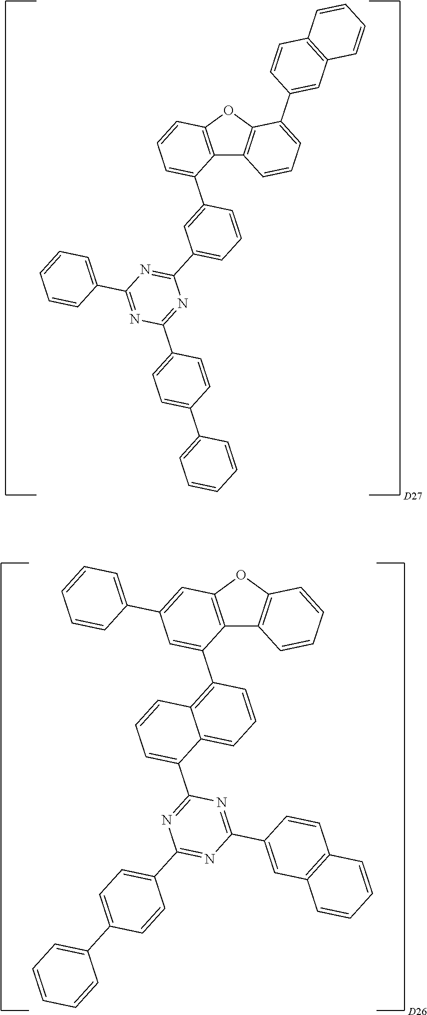

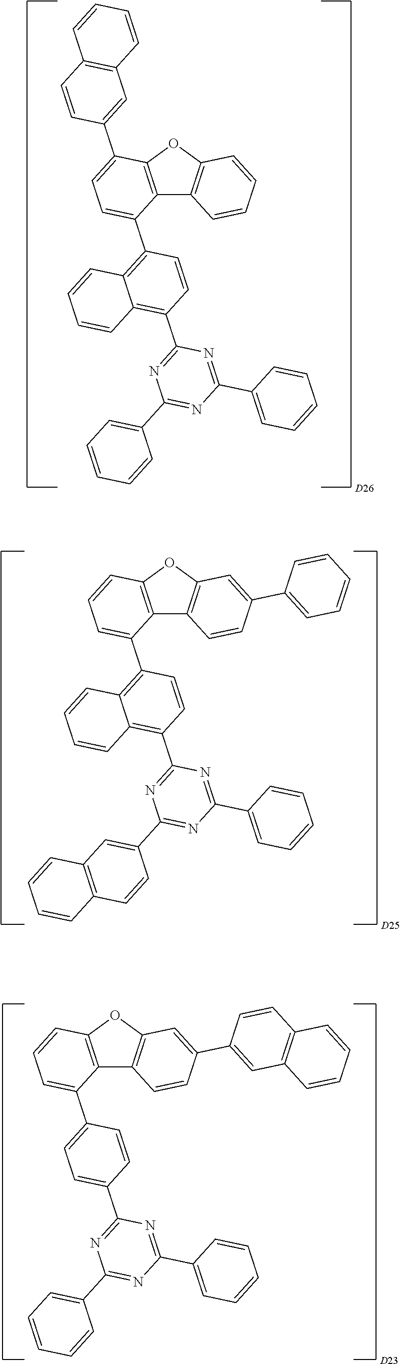

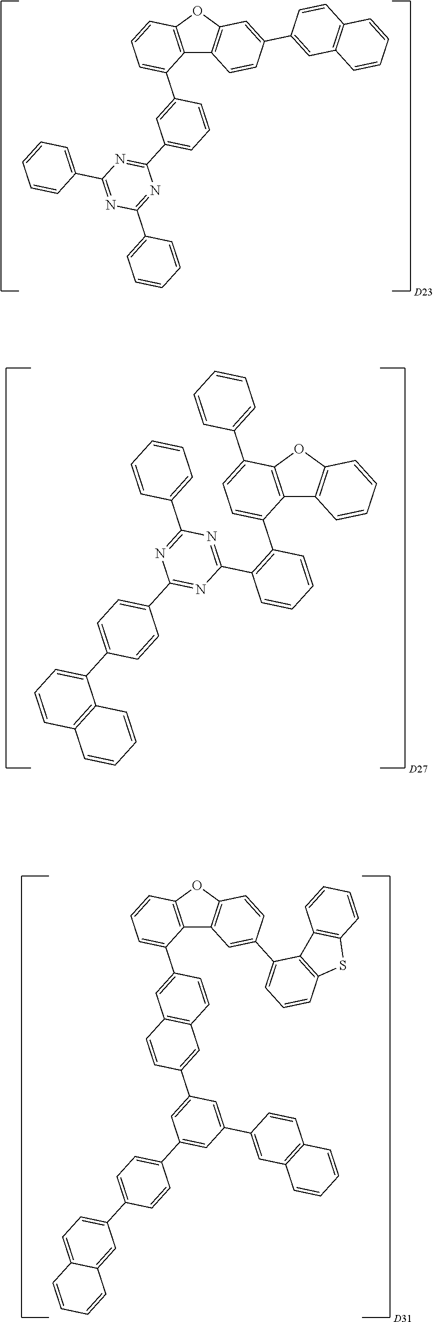

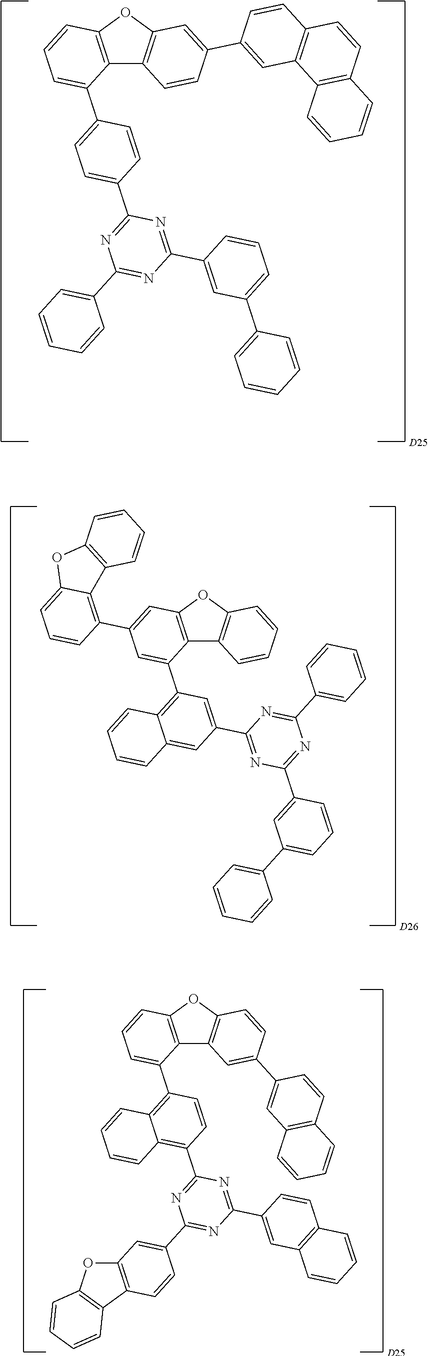

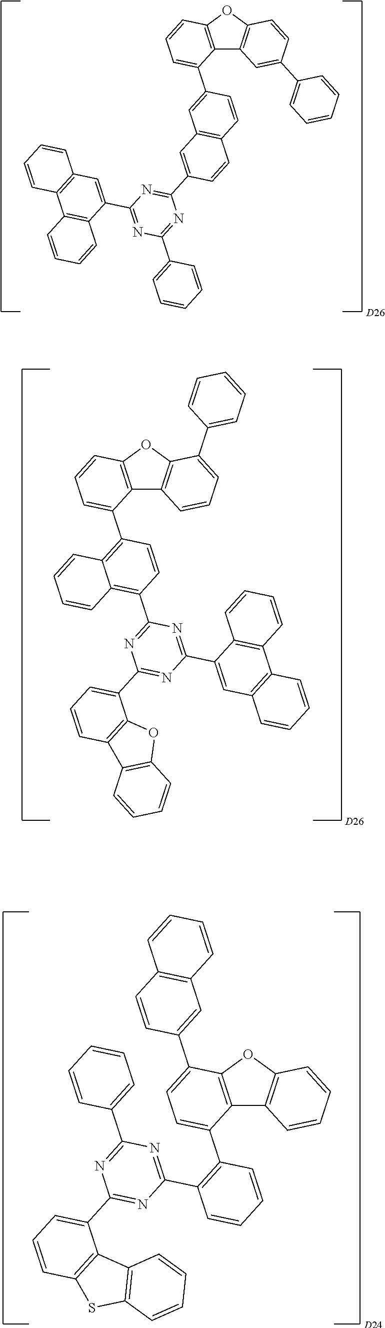

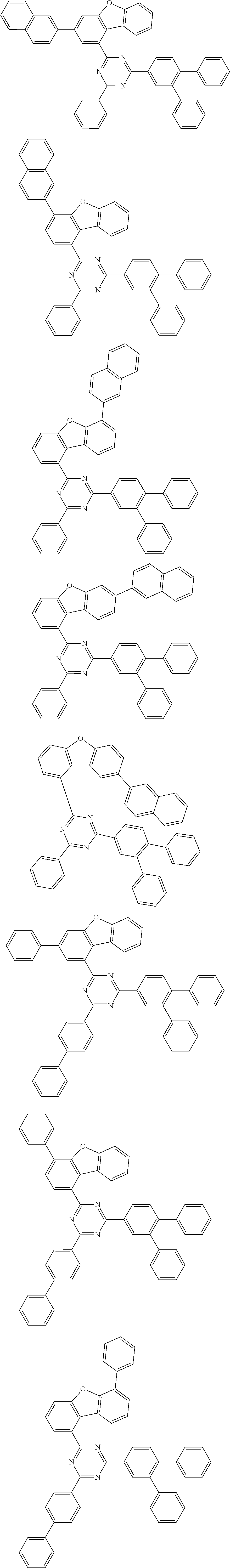

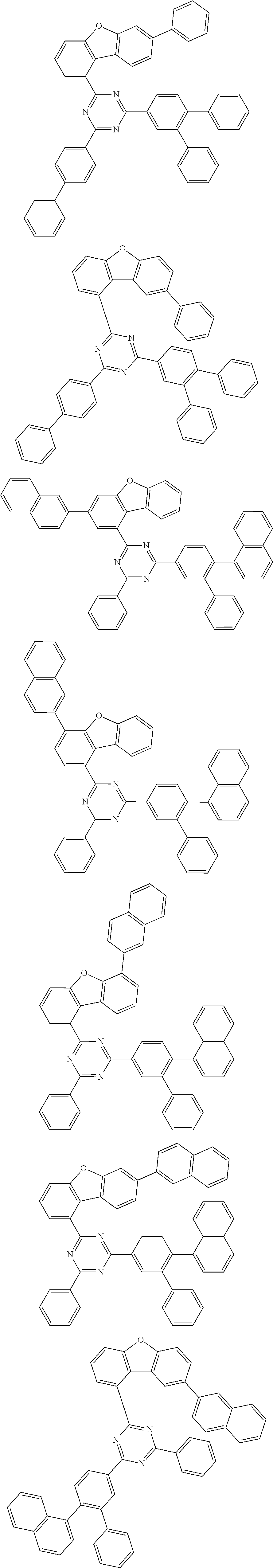

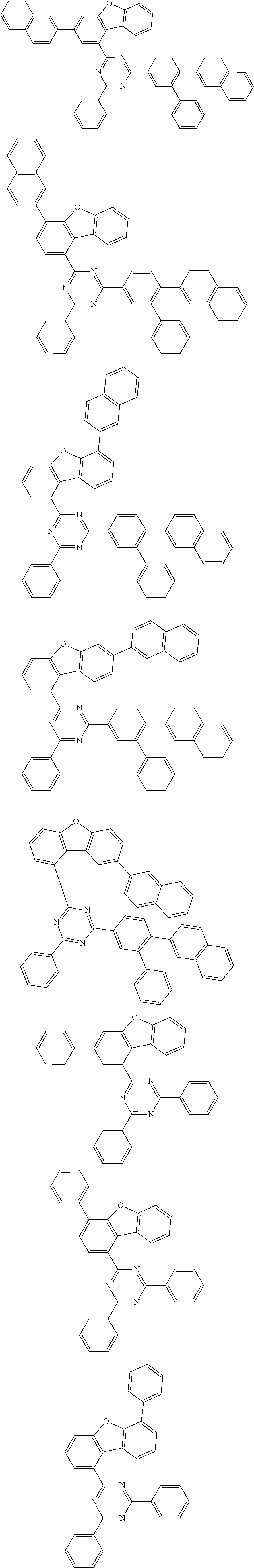

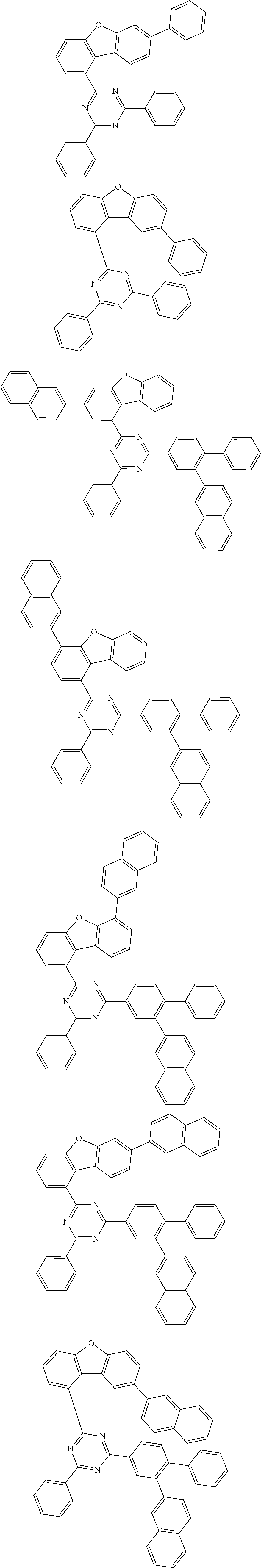

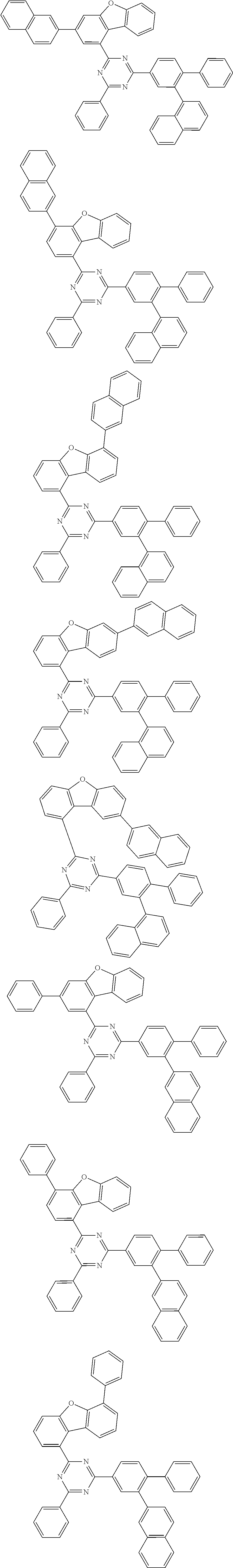

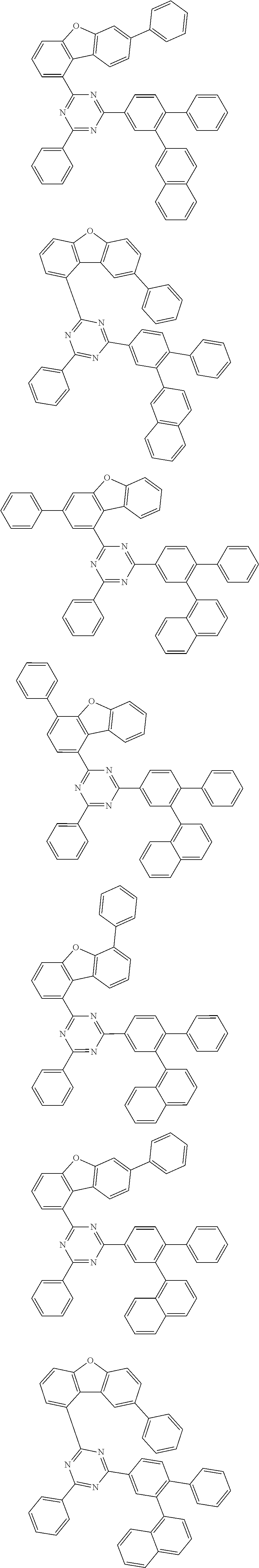

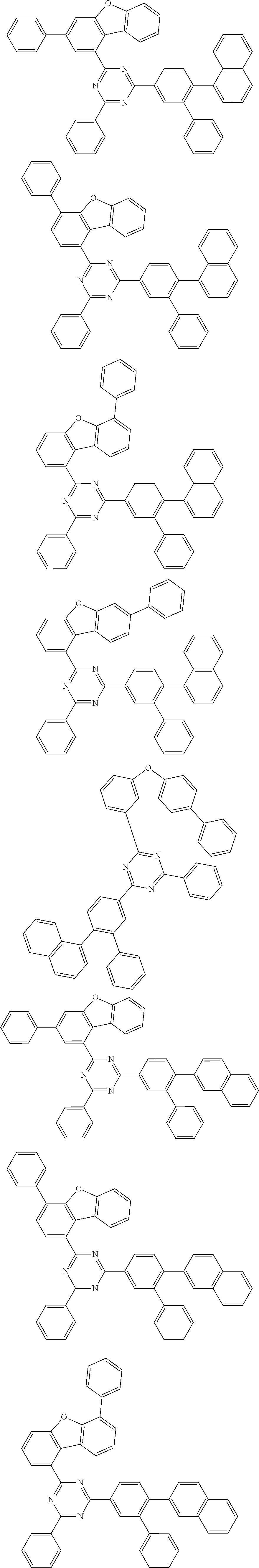

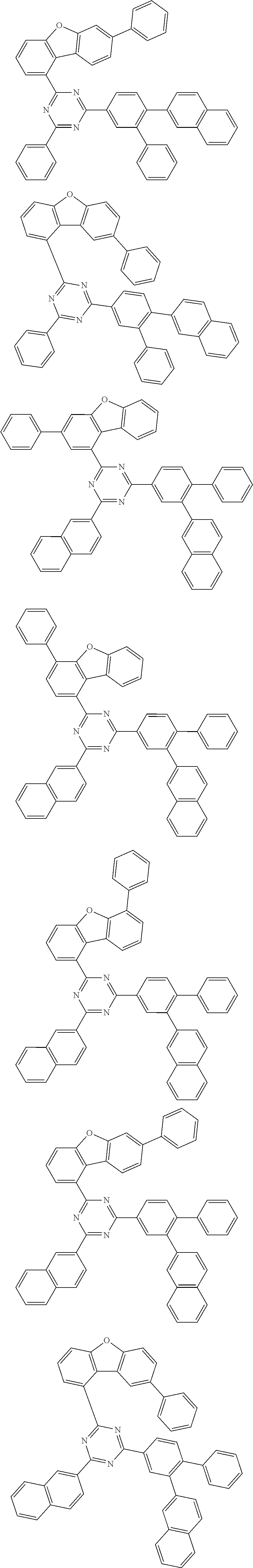

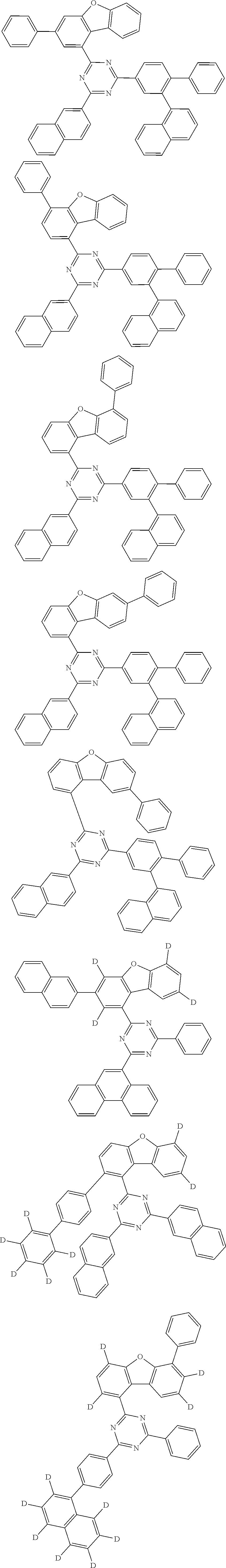

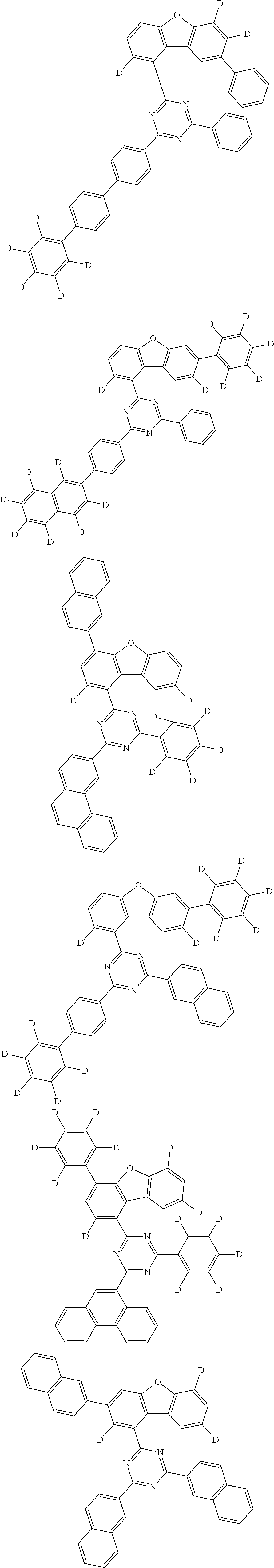

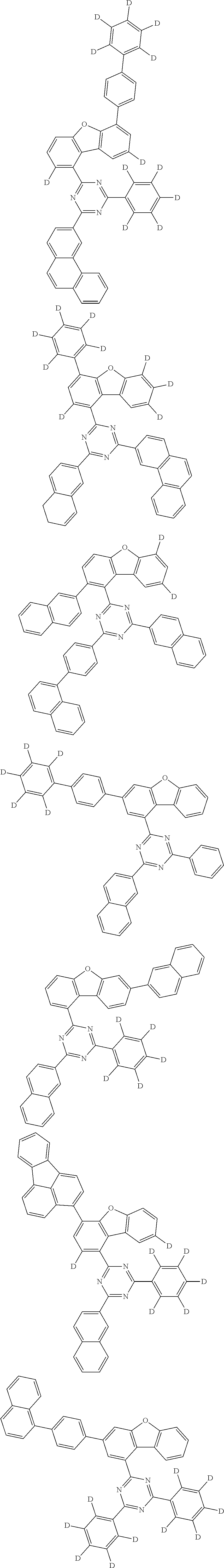

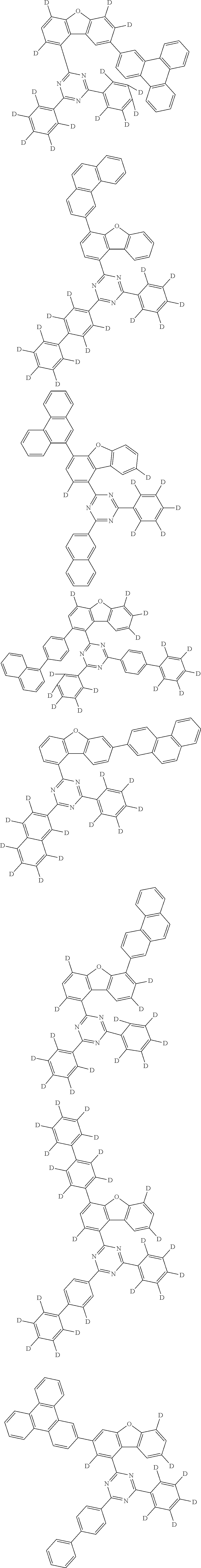

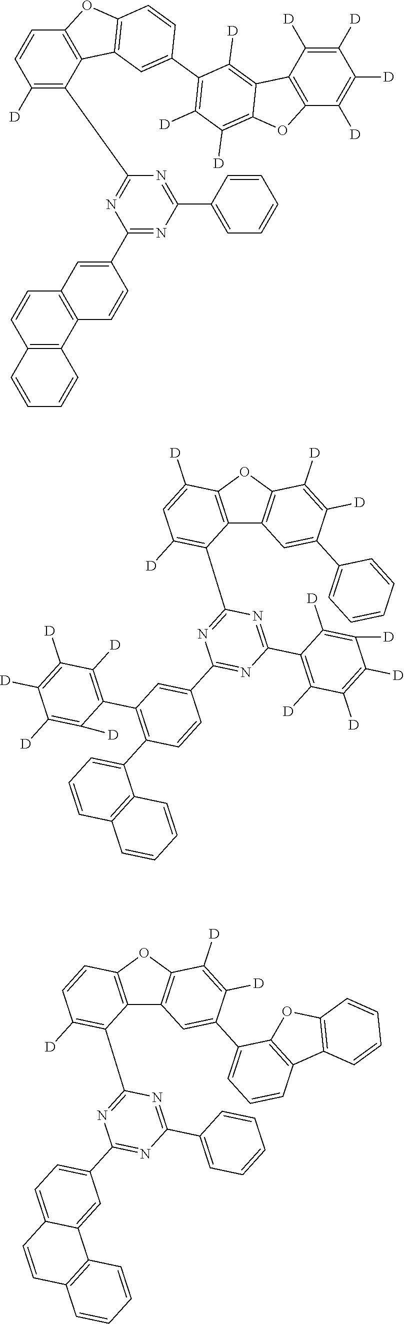 [SMILES: [2H]c1ccc2oc3ccc(-c4c([2H])c([2H])c5oc6c([2H])c([2H])c([2H])c([2H])c6c5c4[2H])cc3c2c1-c1nc(-c2ccccc2)nc(-c2ccc3c(ccc4ccccc43)c2)n1 ; [2H]c1cc([2H])c2oc3c([2H])c([2H])c(-c4ccccc4)cc3c2c1-c1nc(-c2ccc(-c3cccc4ccccc34)c(-c3c([2H])c([2H])c([2H])c([2H])c3[2H])c2)nc(-c2c([2H])c([2H])c([2H])c([2H])c2[2H])n1 ; [2H]c1ccc2oc3c([2H])c([2H])c(-c4cccc5c4oc4ccccc45)cc3c2c1-c1nc(-c2ccccc2)nc(-c2ccc3ccc4ccccc4c3c2)n1]

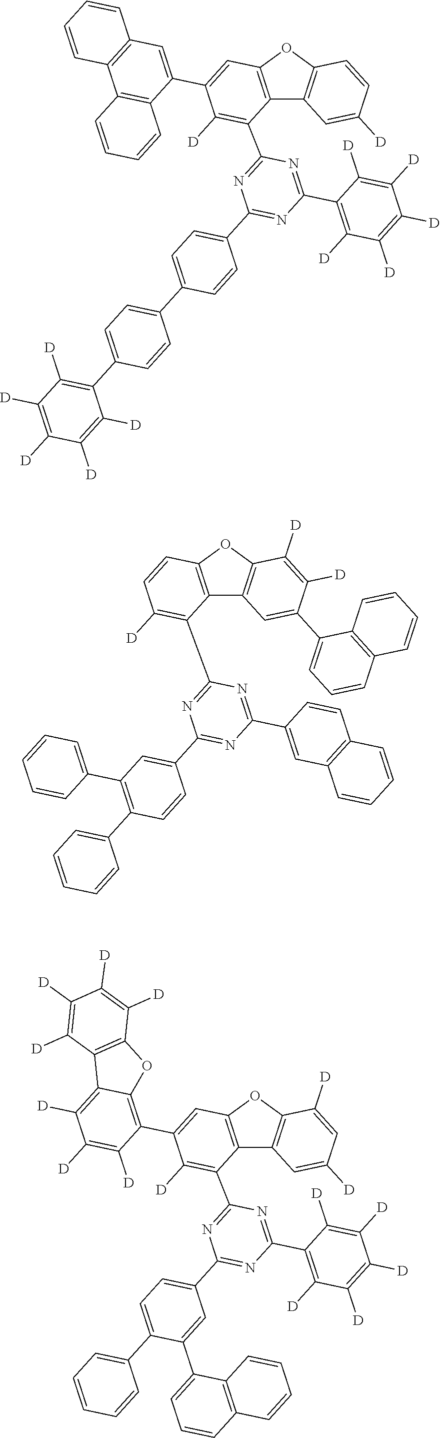 [SMILES: [2H]c1ccc2oc3cc(-c4cc5ccccc5c5ccccc45)c([2H])c(-c4nc(-c5ccc(-c6ccc(-c7c([2H])c([2H])c([2H])c([2H])c7[2H])cc6)cc5)nc(-c5c([2H])c([2H])c([2H])c([2H])c5[2H])n4)c3c2c1 ; [2H]c1ccc2oc3c([2H])c([2H])c(-c4cccc5ccccc45)cc3c2c1-c1nc(-c2ccc(-c3ccccc3)c(-c3ccccc3)c2)nc(-c2ccc3ccccc3c2)n1 ; [2H]c1cc([2H])c2oc3cc(-c4c([2H])c([2H])c([2H])c5c4oc4c([2H])c([2H])c([2H])c([2H])c45)c([2H])c(-c4nc(-c5ccc(-c6ccccc6)c(-c6cccc7ccccc67)c5)nc(-c5c([2H])c([2H])c([2H])c([2H])c5[2H])n4)c3c2c1]

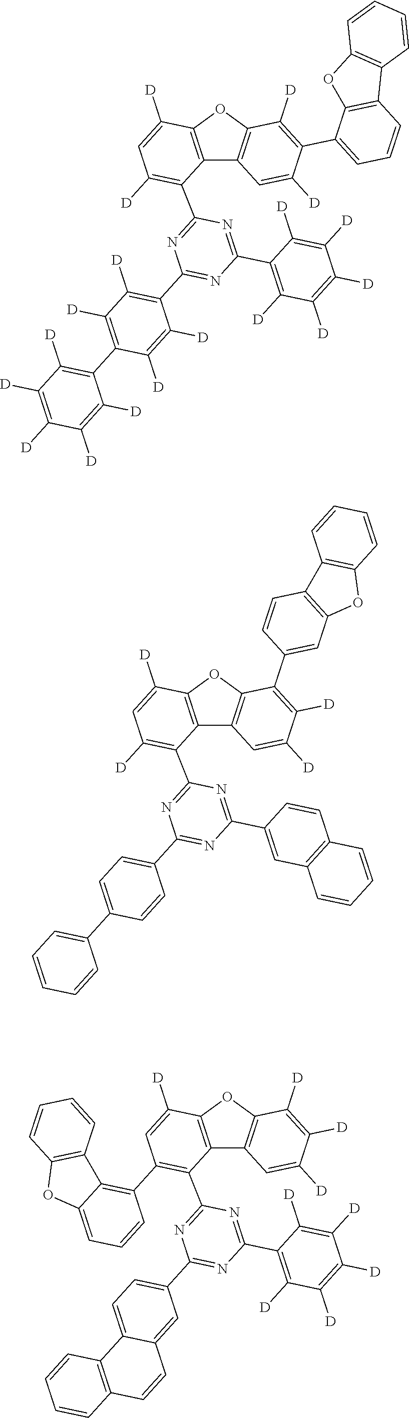 [SMILES: [2H]c1cc2c(oc3c([2H])cc([2H])c(-c4nc(-c5c([2H])c([2H])c([2H])c([2H])c5[2H])nc(-c5c([2H])c([2H])c(-c6c([2H])c([2H])c([2H])c([2H])c6[2H])c([2H])c5[2H])n4)c32)c([2H])c1-c1cccc2c1oc1ccccc12 ; [2H]c1cc2c(oc3c([2H])cc([2H])c(-c4nc(-c5ccc(-c6ccccc6)cc5)nc(-c5ccc6ccccc6c5)n4)c32)c(-c2ccc3c(c2)oc2ccccc23)c1[2H] ; [2H]c1cc2c(oc3c([2H])cc(-c4cccc5oc6ccccc6c45)c(-c4nc(-c5ccc6c(ccc7ccccc76)c5)nc(-c5c([2H])c([2H])c([2H])c([2H])c5[2H])n4)c32)c([2H])c1[2H]]

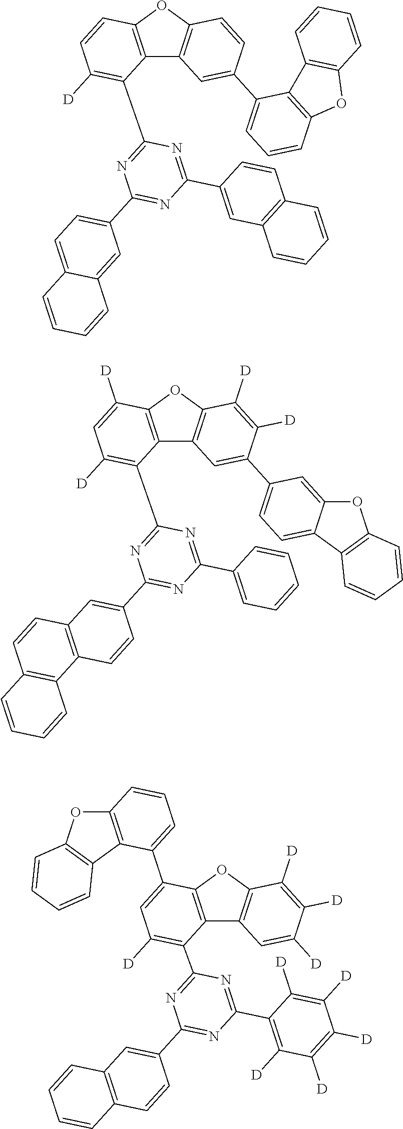

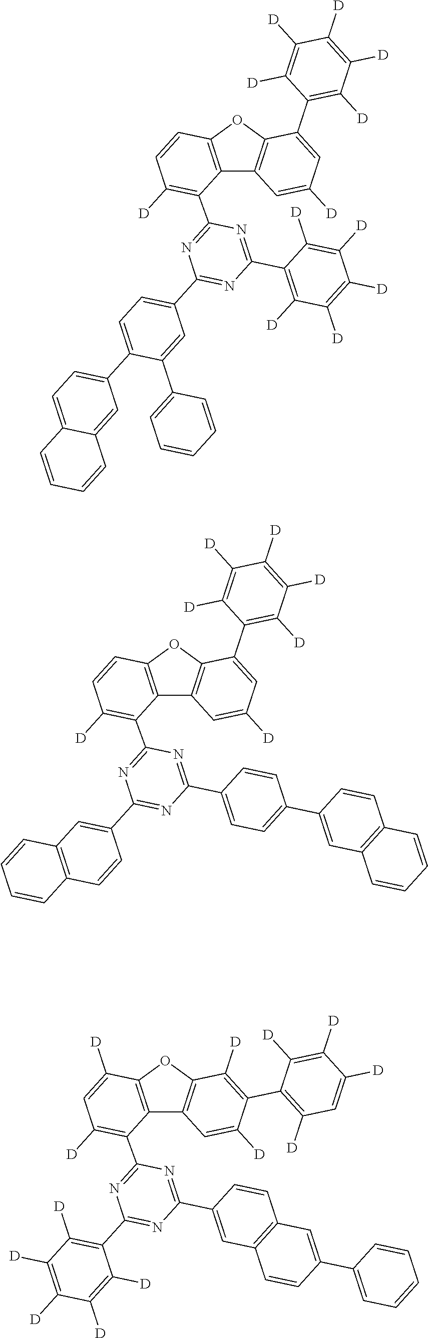 [SMILES: [2H]c1cc(-c2c([2H])c([2H])c([2H])c([2H])c2[2H])c2oc3ccc([2H])c(-c4nc(-c5ccc(-c6ccc7ccccc7c6)c(-c6ccccc6)c5)nc(-c5c([2H])c([2H])c([2H])c([2H])c5[2H])n4)c3c2c1 ; [2H]c1cc(-c2c([2H])c([2H])c([2H])c([2H])c2[2H])c2oc3ccc([2H])c(-c4nc(-c5ccc(-c6ccc7ccccc7c6)cc5)nc(-c5ccc6ccccc6c5)n4)c3c2c1 ; [2H]c1cc([2H])c(-c2c([2H])cc3c(oc4c([2H])cc([2H])c(-c5nc(-c6ccc7cc(-c8ccccc8)ccc7c6)nc(-c6c([2H])c([2H])c([2H])c([2H])c6[2H])n5)c43)c2[2H])c([2H])c1[2H]]

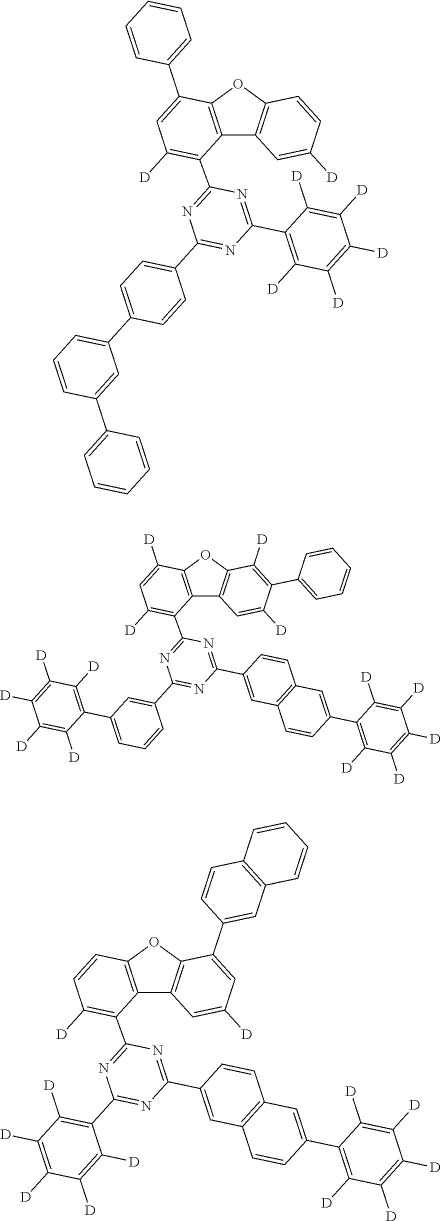 [SMILES: [2H]c1ccc2oc3c(-c4ccccc4)cc([2H])c(-c4nc(-c5ccc(-c6cccc(-c7ccccc7)c6)cc5)nc(-c5c([2H])c([2H])c([2H])c([2H])c5[2H])n4)c3c2c1 ; [2H]c1cc2c(oc3c([2H])cc([2H])c(-c4nc(-c5cccc(-c6c([2H])c([2H])c([2H])c([2H])c6[2H])c5)nc(-c5ccc6cc(-c7c([2H])c([2H])c([2H])c([2H])c7[2H])ccc6c5)n4)c32)c([2H])c1-c1ccccc1 ; [2H]c1cc(-c2ccc3ccccc3c2)c2oc3ccc([2H])c(-c4nc(-c5ccc6cc(-c7c([2H])c([2H])c([2H])c([2H])c7[2H])ccc6c5)nc(-c5c([2H])c([2H])c([2H])c([2H])c5[2H])n4)c3c2c1]

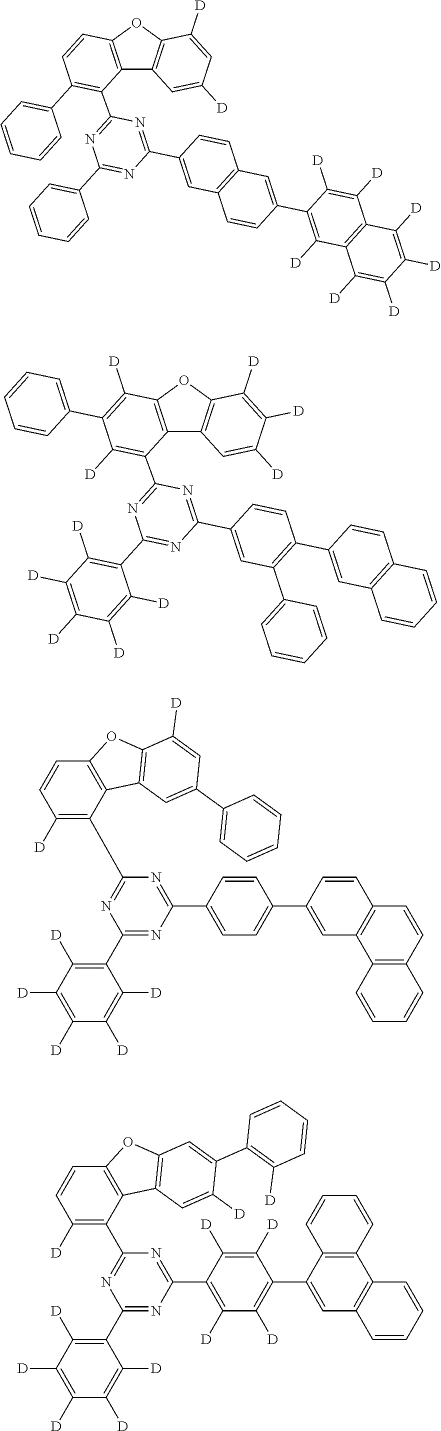

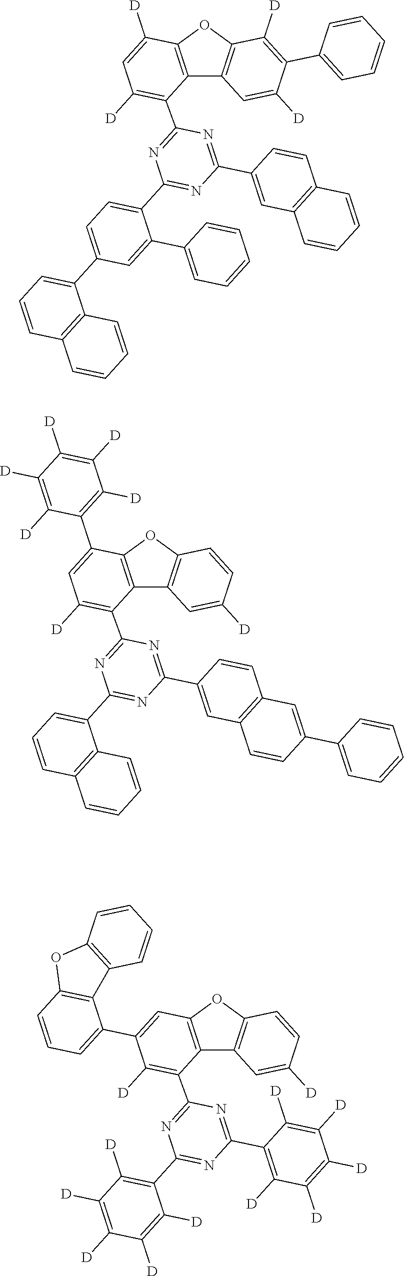 [SMILES: [2H]c1cc2c(oc3c([2H])cc([2H])c(-c4nc(-c5ccc6ccccc6c5)nc(-c5ccc(-c6cccc7ccccc67)cc5-c5ccccc5)n4)c32)c([2H])c1-c1ccccc1 ; [2H]c1ccc2oc3c(-c4c([2H])c([2H])c([2H])c([2H])c4[2H])cc([2H])c(-c4nc(-c5ccc6cc(-c7ccccc7)ccc6c5)nc(-c5cccc6ccccc56)n4)c3c2c1 ; [2H]c1ccc2oc3cc(-c4cccc5oc6ccccc6c45)c([2H])c(-c4nc(-c5c([2H])c([2H])c([2H])c([2H])c5[2H])nc(-c5c([2H])c([2H])c([2H])c([2H])c5[2H])n4)c3c2c1]

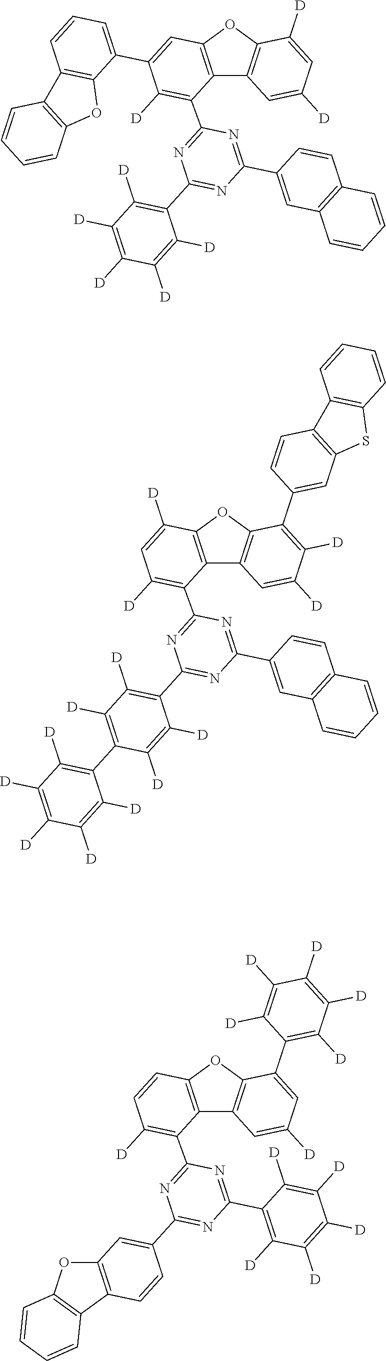 [SMILES: [2H]c1cc([2H])c2oc3cc(-c4cccc5c4oc4ccccc45)c([2H])c(-c4nc(-c5ccc6ccccc6c5)nc(-c5c([2H])c([2H])c([2H])c([2H])c5[2H])n4)c3c2c1 ; [2H]c1cc2c(oc3c([2H])cc([2H])c(-c4nc(-c5ccc6ccccc6c5)nc(-c5c([2H])c([2H])c(-c6c([2H])c([2H])c([2H])c([2H])c6[2H])c([2H])c5[2H])n4)c32)c(-c2ccc3c(c2)sc2ccccc23)c1[2H] ; [2H]c1cc(-c2c([2H])c([2H])c([2H])c([2H])c2[2H])c2oc3ccc([2H])c(-c4nc(-c5ccc6c(c5)oc5ccccc56)nc(-c5c([2H])c([2H])c([2H])c([2H])c5[2H])n4)c3c2c1]

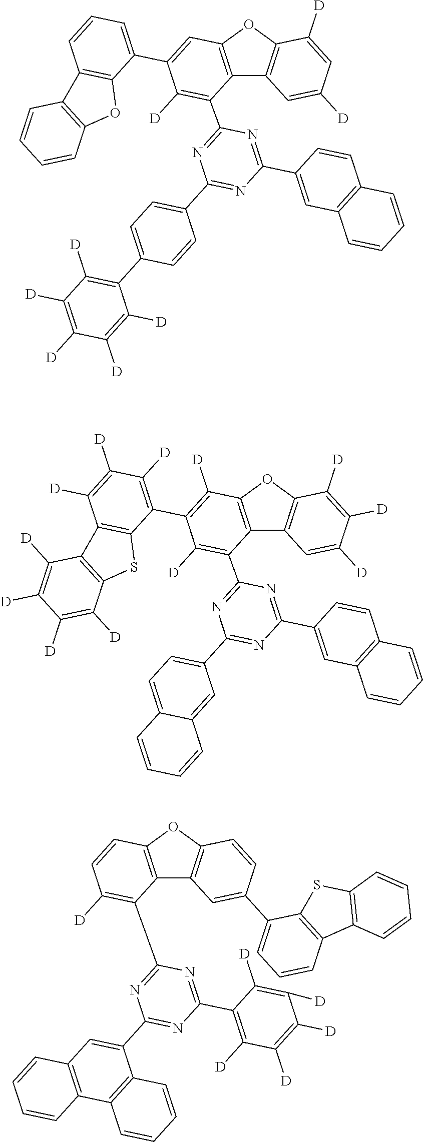 [SMILES: [2H]c1cc([2H])c2oc3cc(-c4cccc5c4oc4ccccc45)c([2H])c(-c4nc(-c5ccc(-c6c([2H])c([2H])c([2H])c([2H])c6[2H])cc5)nc(-c5ccc6ccccc6c5)n4)c3c2c1 ; [2H]c1cc2c(oc3c([2H])c(-c4c([2H])c([2H])c([2H])c5c4sc4c([2H])c([2H])c([2H])c([2H])c45)c([2H])c(-c4nc(-c5ccc6ccccc6c5)nc(-c5ccc6ccccc6c5)n4)c32)c([2H])c1[2H] ; [2H]c1ccc2oc3ccc(-c4cccc5c4sc4ccccc45)cc3c2c1-c1nc(-c2c([2H])c([2H])c([2H])c([2H])c2[2H])nc(-c2cc3ccccc3c3ccccc23)n1]

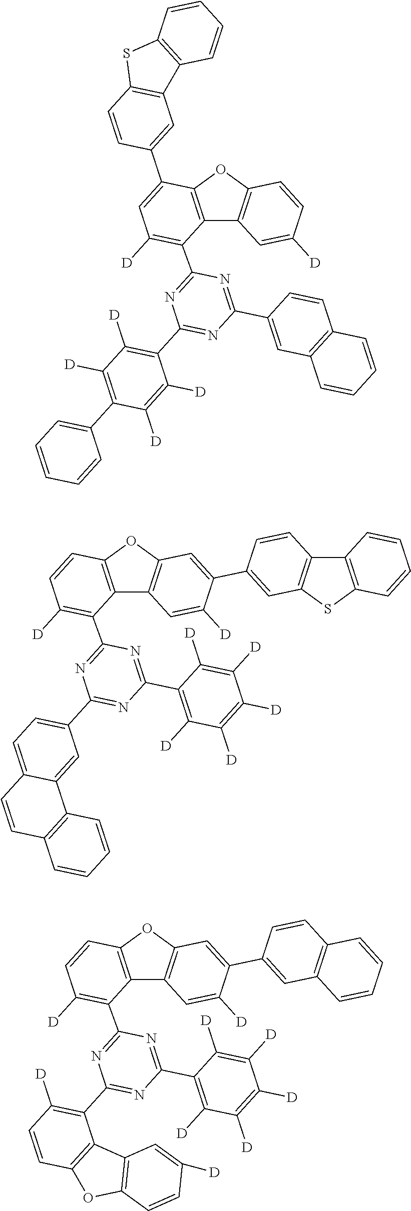 [SMILES: [2H]c1ccc2oc3c(-c4ccc5sc6ccccc6c5c4)cc([2H])c(-c4nc(-c5ccc6ccccc6c5)nc(-c5c([2H])c([2H])c(-c6ccccc6)c([2H])c5[2H])n4)c3c2c1 ; [2H]c1cc2c(cc1-c1ccc3c(c1)sc1ccccc13)oc1ccc([2H])c(-c3nc(-c4ccc5ccc6ccccc6c5c4)nc(-c4c([2H])c([2H])c([2H])c([2H])c4[2H])n3)c12 ; [2H]c1ccc2oc3ccc([2H])c(-c4nc(-c5c([2H])c([2H])c([2H])c([2H])c5[2H])nc(-c5c([2H])ccc6oc7cc(-c8ccc9ccccc9c8)c([2H])cc7c56)n4)c3c2c1]

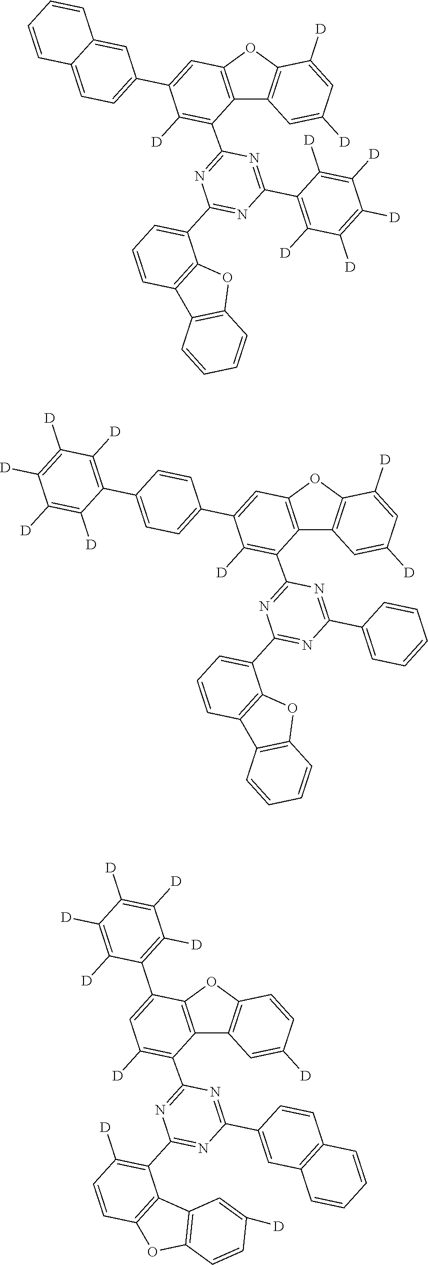 [SMILES: [2H]c1cc([2H])c2oc3cc(-c4ccc5ccccc5c4)c([2H])c(-c4nc(-c5c([2H])c([2H])c([2H])c([2H])c5[2H])nc(-c5cccc6c5oc5ccccc56)n4)c3c2c1 ; [2H]c1cc([2H])c2oc3cc(-c4ccc(-c5c([2H])c([2H])c([2H])c([2H])c5[2H])cc4)c([2H])c(-c4nc(-c5ccccc5)nc(-c5cccc6c5oc5ccccc56)n4)c3c2c1 ; [2H]c1ccc2oc3ccc([2H])c(-c4nc(-c5ccc6ccccc6c5)nc(-c5c([2H])cc(-c6c([2H])c([2H])c([2H])c([2H])c6[2H])c6oc7ccc([2H])cc7c56)n4)c3c2c1]

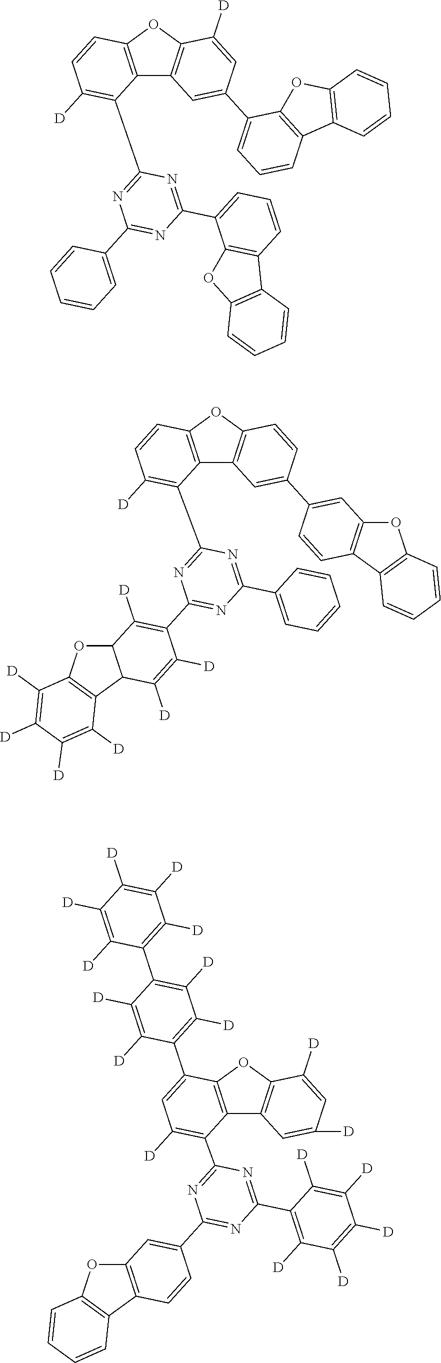 [SMILES: [2H]c1ccc2oc3c([2H])cc(-c4cccc5c4oc4ccccc45)cc3c2c1-c1nc(-c2ccccc2)nc(-c2cccc3c2oc2ccccc23)n1 ; [2H]C1=C([2H])C2c3c([2H])c([2H])c([2H])c([2H])c3OC2C([2H])=C1c1nc(-c2ccccc2)nc(-c2c([2H])ccc3oc4ccc(-c5ccc6c(c5)oc5ccccc56)cc4c23)n1 ; [2H]c1cc([2H])c2oc3c(-c4c([2H])c([2H])c(-c5c([2H])c([2H])c([2H])c([2H])c5[2H])c([2H])c4[2H])cc([2H])c(-c4nc(-c5ccc6c(c5)oc5ccccc56)nc(-c5c([2H])c([2H])c([2H])c([2H])c5[2H])n4)c3c2c1]

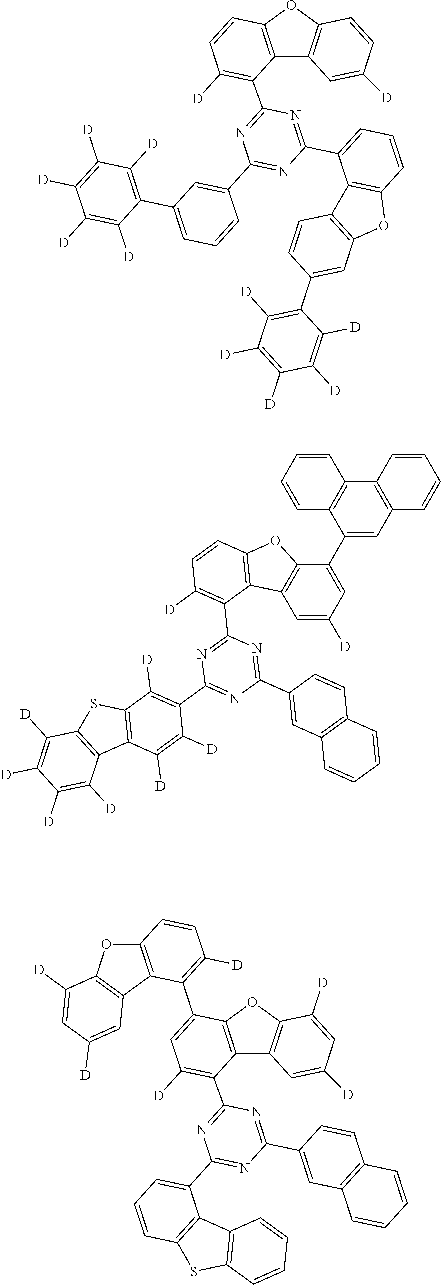 [SMILES: [2H]c1ccc2oc3ccc([2H])c(-c4nc(-c5cccc(-c6c([2H])c([2H])c([2H])c([2H])c6[2H])c5)nc(-c5cccc6oc7cc(-c8c([2H])c([2H])c([2H])c([2H])c8[2H])ccc7c56)n4)c3c2c1 ; [2H]c1cc(-c2cc3ccccc3c3ccccc23)c2oc3ccc([2H])c(-c4nc(-c5ccc6ccccc6c5)nc(-c5c([2H])c([2H])c6c(sc7c([2H])c([2H])c([2H])c([2H])c76)c5[2H])n4)c3c2c1 ; [2H]c1cc([2H])c2oc3ccc([2H])c(-c4cc([2H])c(-c5nc(-c6ccc7ccccc7c6)nc(-c6cccc7sc8ccccc8c67)n5)c5c4oc4c([2H])cc([2H])cc45)c3c2c1]

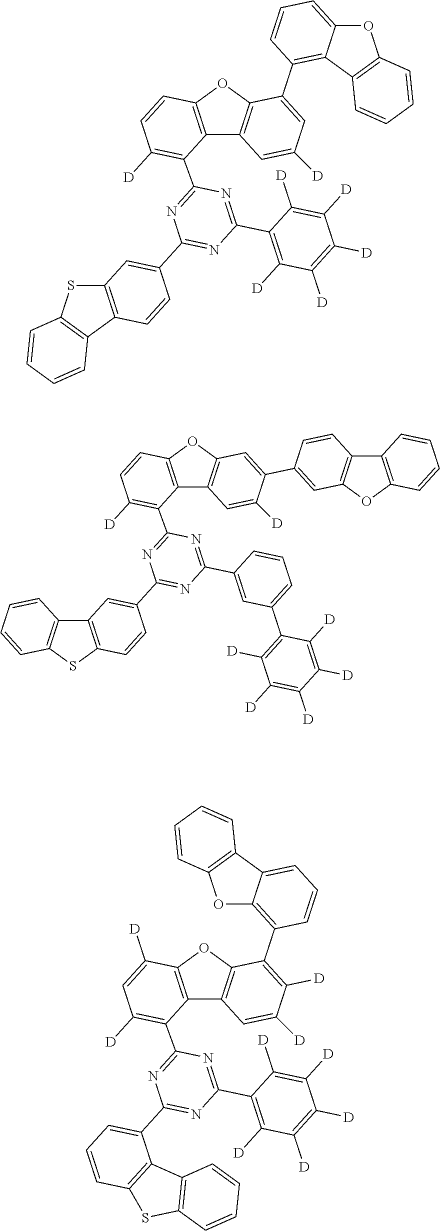 [SMILES: [2H]c1cc(-c2cccc3oc4ccccc4c23)c2oc3ccc([2H])c(-c4nc(-c5ccc6c(c5)sc5ccccc56)nc(-c5c([2H])c([2H])c([2H])c([2H])c5[2H])n4)c3c2c1 ; [2H]c1cc2c(cc1-c1ccc3c(c1)oc1ccccc13)oc1ccc([2H])c(-c3nc(-c4cccc(-c5c([2H])c([2H])c([2H])c([2H])c5[2H])c4)nc(-c4ccc5sc6ccccc6c5c4)n3)c12 ; [2H]c1cc2c(oc3c([2H])cc([2H])c(-c4nc(-c5c([2H])c([2H])c([2H])c([2H])c5[2H])nc(-c5cccc6sc7ccccc7c56)n4)c32)c(-c2cccc3c2oc2ccccc23)c1[2H]]

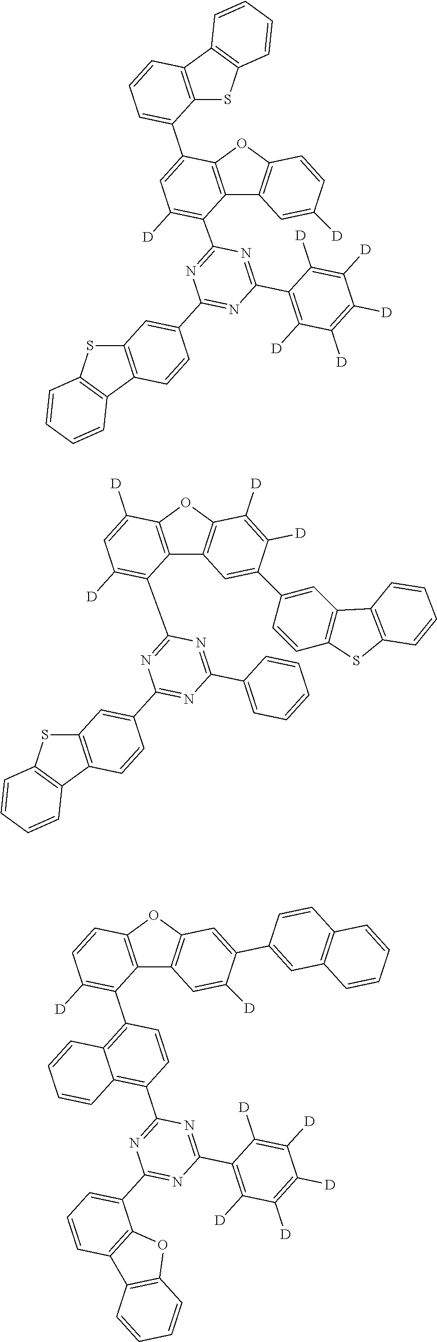 [SMILES: [2H]c1ccc2oc3c(-c4cccc5c4sc4ccccc45)cc([2H])c(-c4nc(-c5ccc6c(c5)sc5ccccc56)nc(-c5c([2H])c([2H])c([2H])c([2H])c5[2H])n4)c3c2c1 ; [2H]c1cc([2H])c2oc3c([2H])c([2H])c(-c4ccc5sc6ccccc6c5c4)cc3c2c1-c1nc(-c2ccccc2)nc(-c2ccc3c(c2)sc2ccccc23)n1 ; [2H]c1cc2c(cc1-c1ccc3ccccc3c1)oc1ccc([2H])c(-c3ccc(-c4nc(-c5c([2H])c([2H])c([2H])c([2H])c5[2H])nc(-c5cccc6c5oc5ccccc56)n4)c4ccccc34)c12]

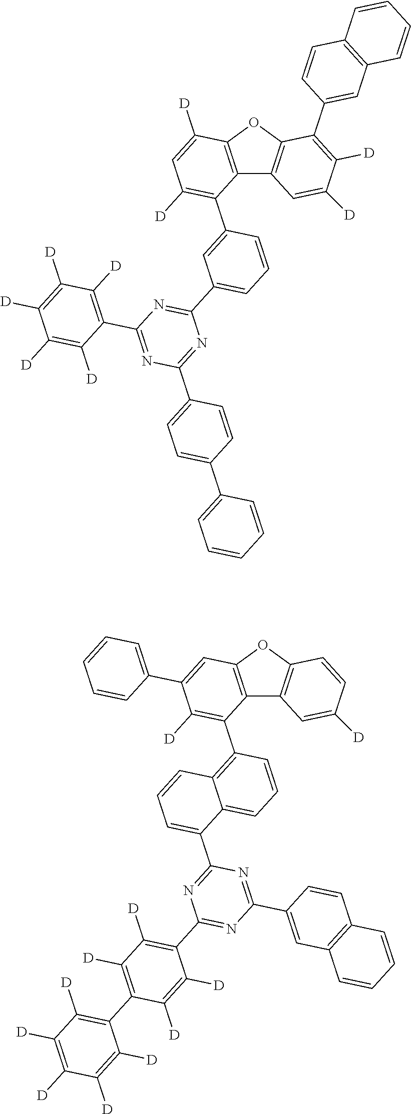 [SMILES: [2H]c1cc2c(oc3c([2H])cc([2H])c(-c4cccc(-c5nc(-c6ccc(-c7ccccc7)cc6)nc(-c6c([2H])c([2H])c([2H])c([2H])c6[2H])n5)c4)c32)c(-c2ccc3ccccc3c2)c1[2H] ; [2H]c1ccc2oc3cc(-c4ccccc4)c([2H])c(-c4cccc5c(-c6nc(-c7ccc8ccccc8c7)nc(-c7c([2H])c([2H])c(-c8c([2H])c([2H])c([2H])c([2H])c8[2H])c([2H])c7[2H])n6)cccc45)c3c2c1]

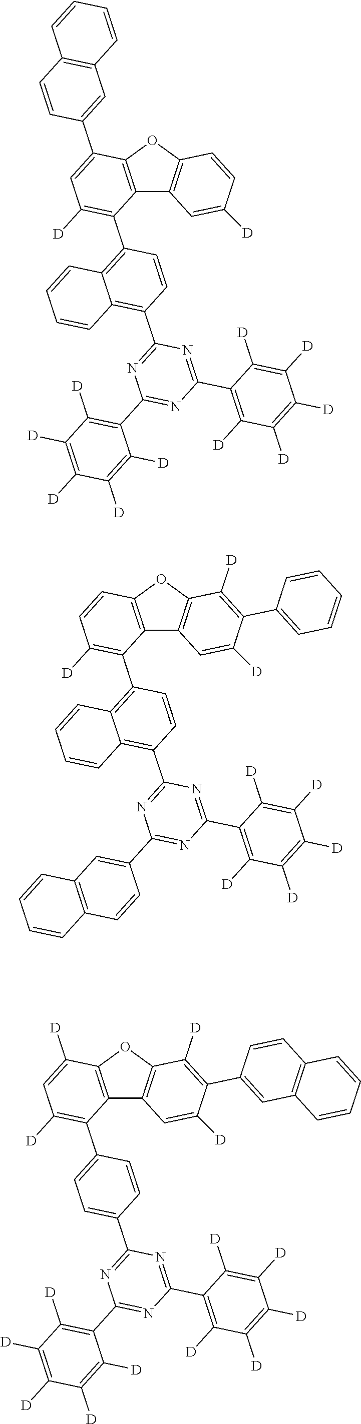 [SMILES: [2H]c1ccc2oc3c(-c4ccc5ccccc5c4)cc([2H])c(-c4ccc(-c5nc(-c6c([2H])c([2H])c([2H])c([2H])c6[2H])nc(-c6c([2H])c([2H])c([2H])c([2H])c6[2H])n5)c5ccccc45)c3c2c1 ; [2H]c1cc2c(oc3ccc([2H])c(-c4ccc(-c5nc(-c6ccc7ccccc7c6)nc(-c6c([2H])c([2H])c([2H])c([2H])c6[2H])n5)c5ccccc45)c32)c([2H])c1-c1ccccc1 ; [2H]c1cc2c(oc3c([2H])cc([2H])c(-c4ccc(-c5nc(-c6c([2H])c([2H])c([2H])c([2H])c6[2H])nc(-c6c([2H])c([2H])c([2H])c([2H])c6[2H])n5)cc4)c32)c([2H])c1-c1ccc2ccccc2c1]

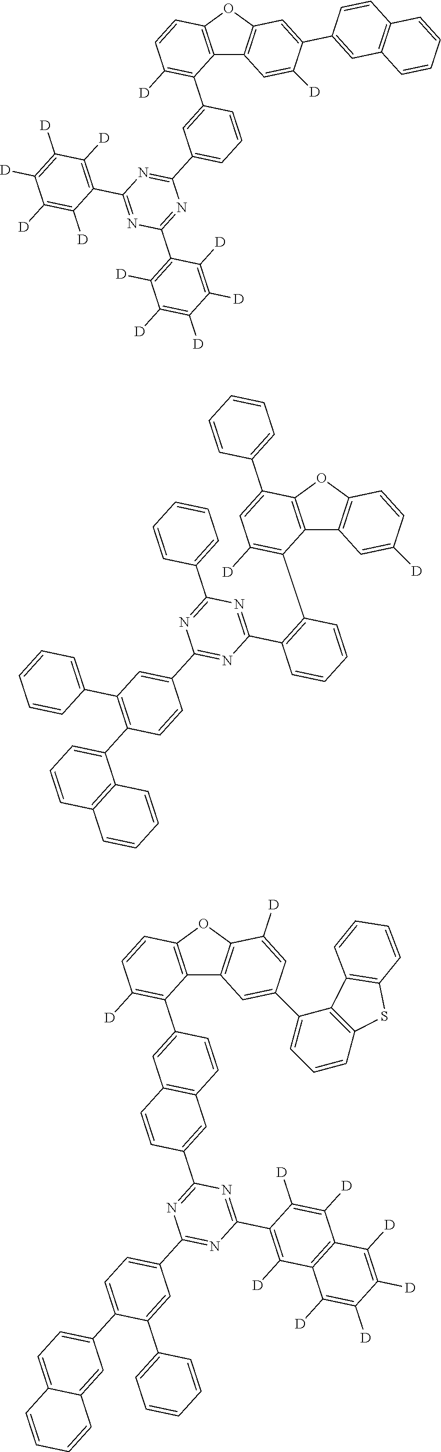 [SMILES: [2H]c1cc2c(cc1-c1ccc3ccccc3c1)oc1ccc([2H])c(-c3cccc(-c4nc(-c5c([2H])c([2H])c([2H])c([2H])c5[2H])nc(-c5c([2H])c([2H])c([2H])c([2H])c5[2H])n4)c3)c12 ; [2H]c1ccc2oc3c(-c4ccccc4)cc([2H])c(-c4ccccc4-c4nc(-c5ccccc5)nc(-c5ccc(-c6cccc7ccccc67)c(-c6ccccc6)c5)n4)c3c2c1 ; [2H]c1ccc2oc3c([2H])cc(-c4cccc5sc6ccccc6c45)cc3c2c1-c1ccc2cc(-c3nc(-c4ccc(-c5ccc6ccccc6c5)c(-c5ccccc5)c4)nc(-c4c([2H])c([2H])c5c([2H])c([2H])c([2H])c([2H])c5c4[2H])n3)ccc2c1]

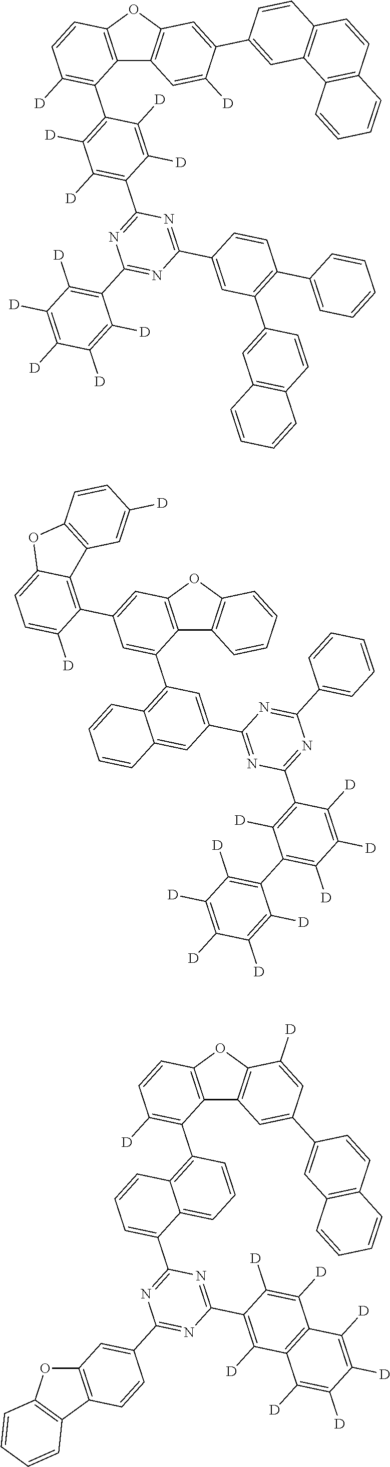 [SMILES: [2H]c1cc2c(cc1-c1ccc3ccc4ccccc4c3c1)oc1ccc([2H])c(-c3c([2H])c([2H])c(-c4nc(-c5ccc(-c6ccccc6)c(-c6ccc7ccccc7c6)c5)nc(-c5c([2H])c([2H])c([2H])c([2H])c5[2H])n4)c([2H])c3[2H])c12 ; [2H]c1ccc2oc3ccc([2H])c(-c4cc(-c5cc(-c6nc(-c7ccccc7)nc(-c7c([2H])c([2H])c([2H])c(-c8c([2H])c([2H])c([2H])c([2H])c8[2H])c7[2H])n6)cc6ccccc56)c5c(c4)oc4ccccc45)c3c2c1 ; [2H]c1ccc2oc3c([2H])cc(-c4ccc5ccccc5c4)cc3c2c1-c1cccc2c(-c3nc(-c4ccc5c(c4)oc4ccccc45)nc(-c4c([2H])c([2H])c5c([2H])c([2H])c([2H])c([2H])c5c4[2H])n3)cccc12]

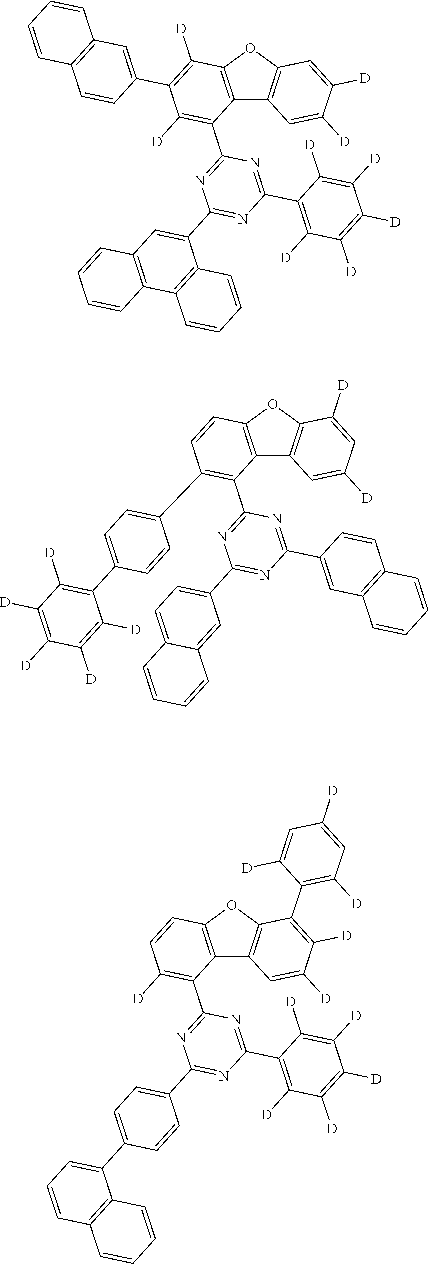 [SMILES: [2H]c1cc2oc3c([2H])c(-c4ccc5ccccc5c4)c([2H])c(-c4nc(-c5c([2H])c([2H])c([2H])c([2H])c5[2H])nc(-c5cc6ccccc6c6ccccc56)n4)c3c2cc1[2H] ; [2H]c1cc([2H])c2oc3ccc(-c4ccc(-c5c([2H])c([2H])c([2H])c([2H])c5[2H])cc4)c(-c4nc(-c5ccc6ccccc6c5)nc(-c5ccc6ccccc6c5)n4)c3c2c1 ; [2H]c1cc([2H])c(-c2c([2H])c([2H])cc3c2oc2ccc([2H])c(-c4nc(-c5ccc(-c6cccc7ccccc67)cc5)nc(-c5c([2H])c([2H])c([2H])c([2H])c5[2H])n4)c23)c([2H])c1]

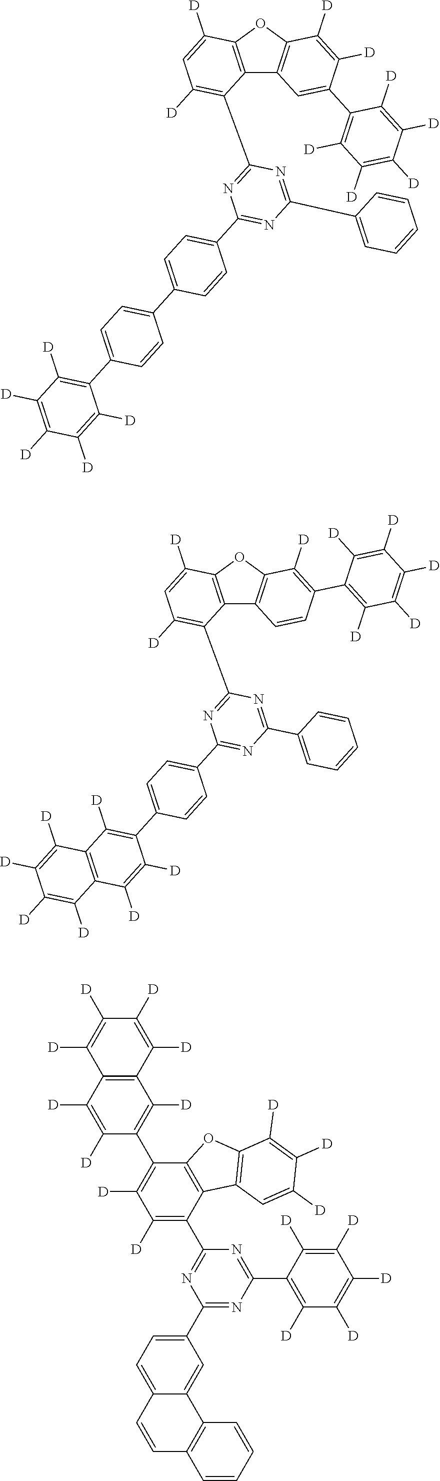

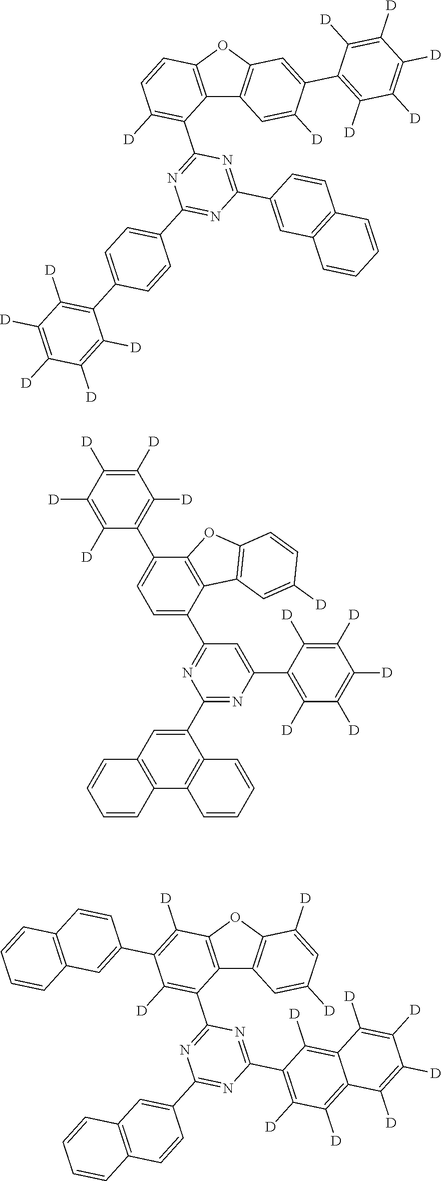

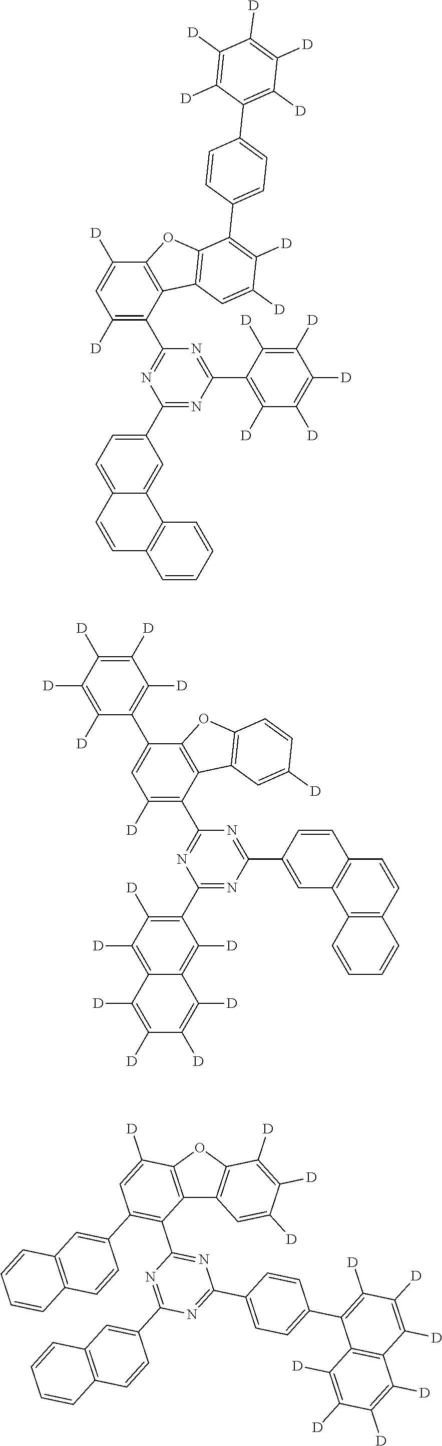 [SMILES: [2H]c1cc2c(oc3c([2H])cc([2H])c(-c4nc(-c5ccc6ccc7ccccc7c6c5)nc(-c5c([2H])c([2H])c([2H])c([2H])c5[2H])n4)c32)c(-c2ccc(-c3c([2H])c([2H])c([2H])c([2H])c3[2H])cc2)c1[2H] ; [2H]c1ccc2oc3c(-c4c([2H])c([2H])c([2H])c([2H])c4[2H])cc([2H])c(-c4nc(-c5ccc6ccc7ccccc7c6c5)nc(-c5c([2H])c([2H])c6c([2H])c([2H])c([2H])c([2H])c6c5[2H])n4)c3c2c1 ; [2H]c1cc2c(oc3c([2H])cc(-c4ccc5ccccc5c4)c(-c4nc(-c5ccc(-c6c([2H])c([2H])c([2H])c7c([2H])c([2H])c([2H])c([2H])c67)cc5)nc(-c5ccc6ccccc6c5)n4)c32)c([2H])c1[2H]]

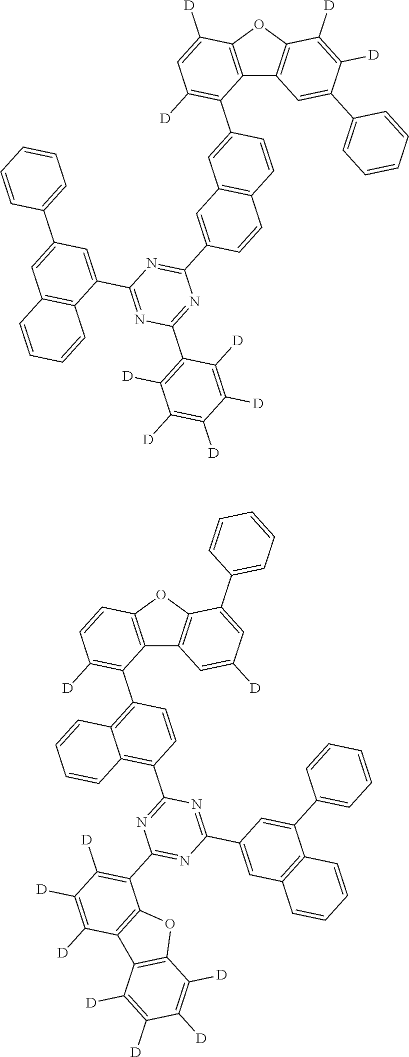 [SMILES: [2H]c1cc([2H])c2oc3c([2H])c([2H])c(-c4ccccc4)cc3c2c1-c1ccc2ccc(-c3nc(-c4c([2H])c([2H])c([2H])c([2H])c4[2H])nc(-c4cc(-c5ccccc5)cc5ccccc45)n3)cc2c1 ; [2H]c1cc(-c2ccccc2)c2oc3ccc([2H])c(-c4ccc(-c5nc(-c6cc(-c7ccccc7)c7ccccc7c6)nc(-c6c([2H])c([2H])c([2H])c7c6oc6c([2H])c([2H])c([2H])c([2H])c67)n5)c5ccccc45)c3c2c1]

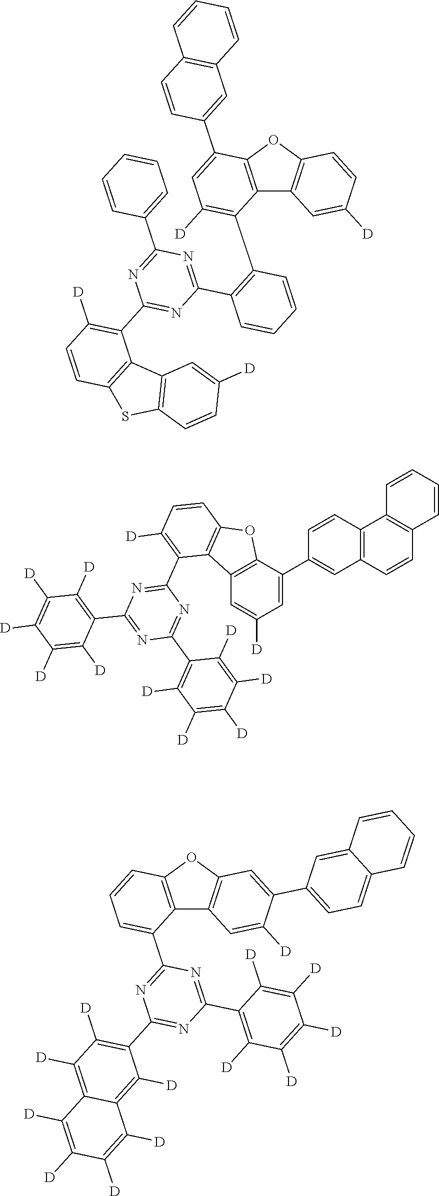 [SMILES: [2H]c1ccc2sc3ccc([2H])c(-c4nc(-c5ccccc5)nc(-c5ccccc5-c5c([2H])cc(-c6ccc7ccccc7c6)c6oc7ccc([2H])cc7c56)n4)c3c2c1 ; [2H]c1cc(-c2ccc3c(ccc4ccccc43)c2)c2oc3ccc([2H])c(-c4nc(-c5c([2H])c([2H])c([2H])c([2H])c5[2H])nc(-c5c([2H])c([2H])c([2H])c([2H])c5[2H])n4)c3c2c1 ; [2H]c1cc2c(cc1-c1ccc3ccccc3c1)oc1cccc(-c3nc(-c4c([2H])c([2H])c([2H])c([2H])c4[2H])nc(-c4c([2H])c([2H])c5c([2H])c([2H])c([2H])c([2H])c5c4[2H])n3)c12]

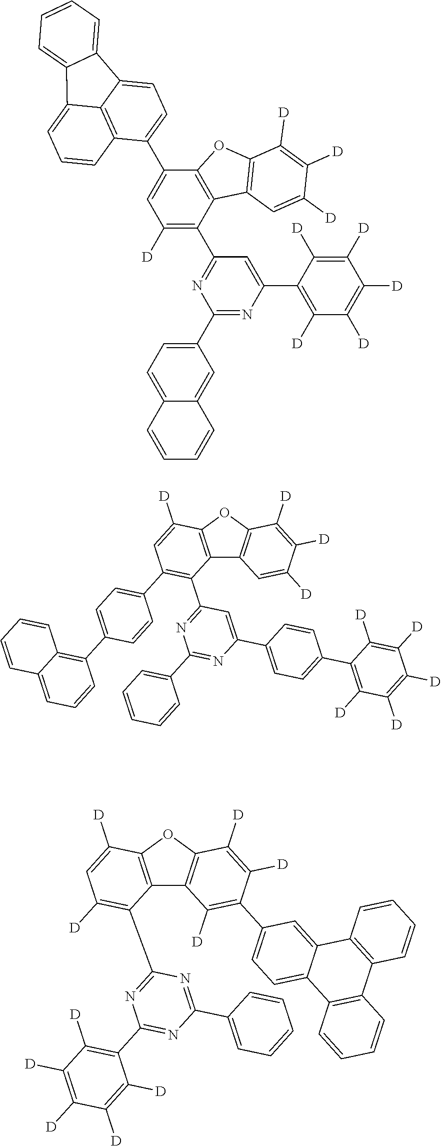

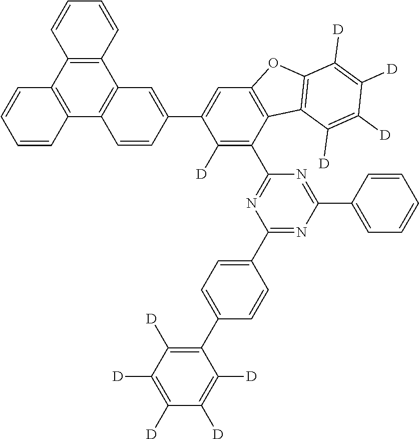 [SMILES: [2H]c1c([2H])c([2H])c(-c2ccc(-c3nc(-c4ccccc4)nc(-c4c([2H])c(-c5ccc6c7ccccc7c7ccccc7c6c5)cc5oc6c([2H])c([2H])c([2H])c([2H])c6c45)n3)cc2)c([2H])c1[2H]]

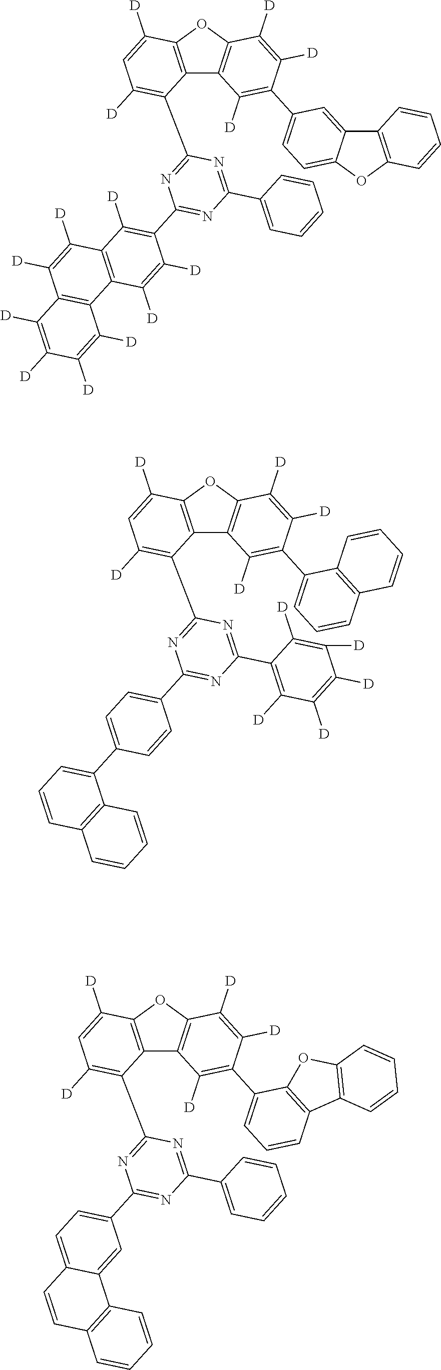 [SMILES: [2H]c1cc([2H])c2oc3c([2H])c([2H])c(-c4ccc5oc6ccccc6c5c4)c([2H])c3c2c1-c1nc(-c2ccccc2)nc(-c2c([2H])c([2H])c3c(c2[2H])c([2H])c([2H])c2c([2H])c([2H])c([2H])c([2H])c23)n1 ; [2H]c1cc([2H])c2oc3c([2H])c([2H])c(-c4cccc5ccccc45)c([2H])c3c2c1-c1nc(-c2ccc(-c3cccc4ccccc34)cc2)nc(-c2c([2H])c([2H])c([2H])c([2H])c2[2H])n1 ; [2H]c1cc([2H])c2oc3c([2H])c([2H])c(-c4cccc5c4oc4ccccc45)c([2H])c3c2c1-c1nc(-c2ccccc2)nc(-c2ccc3ccc4ccccc4c3c2)n1]

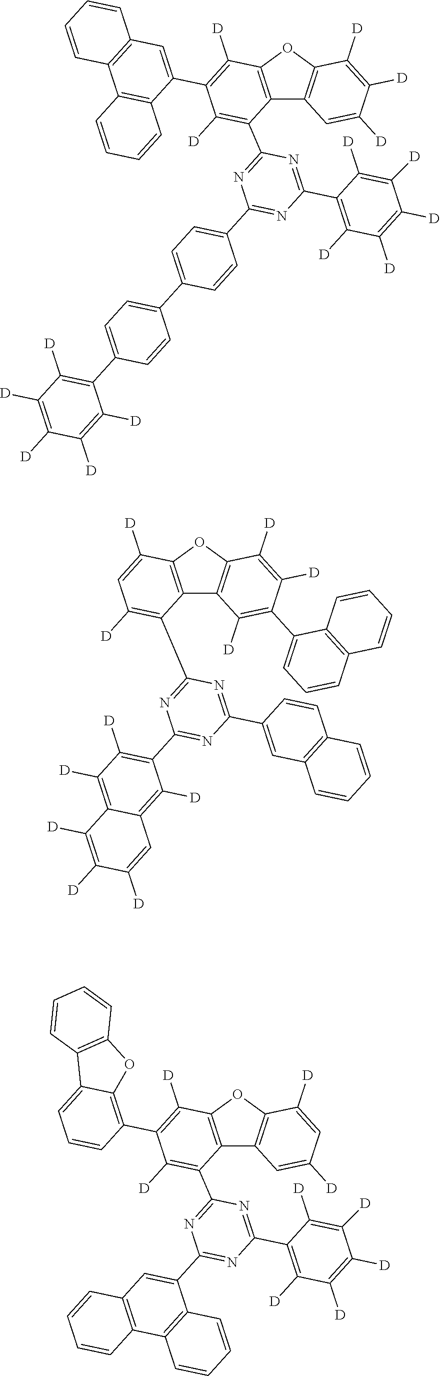 [SMILES: [2H]c1cc2c(oc3c([2H])c(-c4cc5ccccc5c5ccccc45)c([2H])c(-c4nc(-c5ccc(-c6ccc(-c7c([2H])c([2H])c([2H])c([2H])c7[2H])cc6)cc5)nc(-c5c([2H])c([2H])c([2H])c([2H])c5[2H])n4)c32)c([2H])c1[2H] ; [2H]c1cc2c([2H])c(-c3nc(-c4ccc5ccccc5c4)nc(-c4c([2H])cc([2H])c5oc6c([2H])c([2H])c(-c7cccc8ccccc78)c([2H])c6c45)n3)c([2H])c([2H])c2c([2H])c1[2H] ; [2H]c1cc([2H])c2oc3c([2H])c(-c4cccc5c4oc4ccccc45)c([2H])c(-c4nc(-c5c([2H])c([2H])c([2H])c([2H])c5[2H])nc(-c5cc6ccccc6c6ccccc56)n4)c3c2c1]

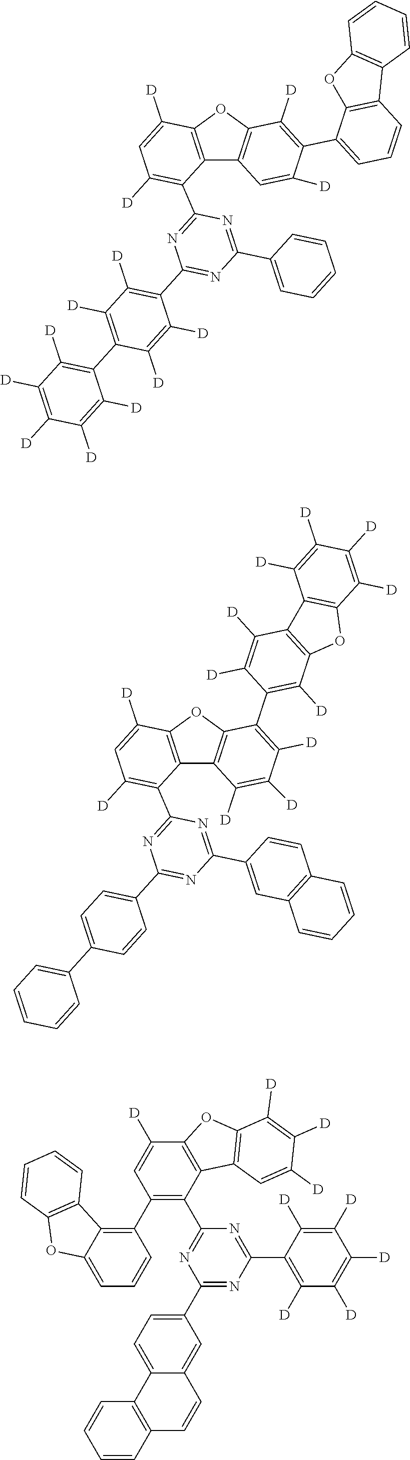 [SMILES: [2H]c1cc2c(oc3c([2H])cc([2H])c(-c4nc(-c5ccccc5)nc(-c5c([2H])c([2H])c(-c6c([2H])c([2H])c([2H])c([2H])c6[2H])c([2H])c5[2H])n4)c32)c([2H])c1-c1cccc2c1oc1ccccc12 ; [2H]c1cc([2H])c2oc3c(-c4c([2H])c([2H])c5c(oc6c([2H])c([2H])c([2H])c([2H])c65)c4[2H])c([2H])c([2H])c([2H])c3c2c1-c1nc(-c2ccc(-c3ccccc3)cc2)nc(-c2ccc3ccccc3c2)n1 ; [2H]c1cc2c(oc3c([2H])cc(-c4cccc5oc6ccccc6c45)c(-c4nc(-c5ccc6c(ccc7ccccc76)c5)nc(-c5c([2H])c([2H])c([2H])c([2H])c5[2H])n4)c32)c([2H])c1[2H]]

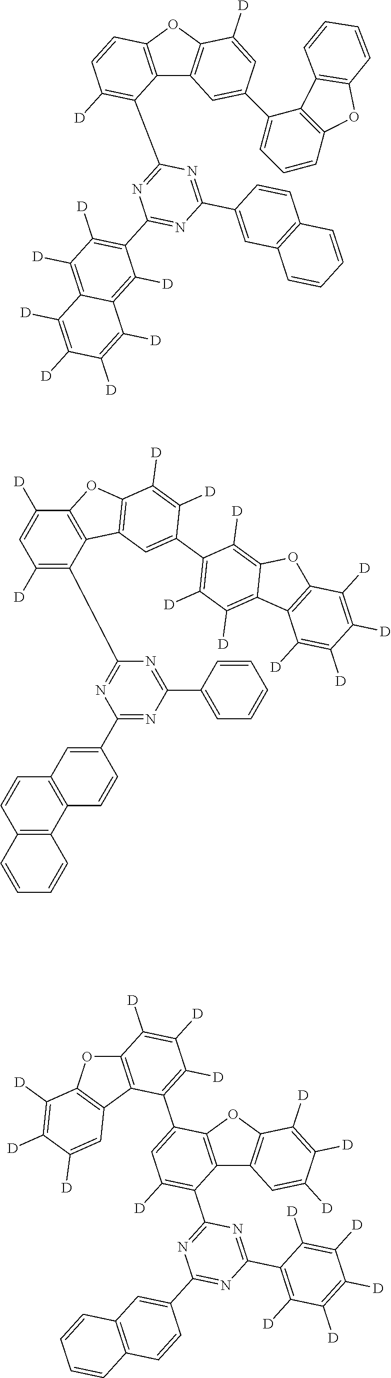 [SMILES: [2H]c1ccc2oc3c([2H])cc(-c4cccc5oc6ccccc6c45)cc3c2c1-c1nc(-c2ccc3ccccc3c2)nc(-c2c([2H])c([2H])c3c([2H])c([2H])c([2H])c([2H])c3c2[2H])n1 ; [2H]c1cc([2H])c2oc3c([2H])c([2H])c(-c4c([2H])c([2H])c5c(oc6c([2H])c([2H])c([2H])c([2H])c65)c4[2H])cc3c2c1-c1nc(-c2ccccc2)nc(-c2ccc3c(ccc4ccccc43)c2)n1 ; [2H]c1cc2c(oc3c(-c4c([2H])c([2H])c([2H])c5oc6c([2H])c([2H])c([2H])cc6c45)cc([2H])c(-c4nc(-c5ccc6ccccc6c5)nc(-c5c([2H])c([2H])c([2H])c([2H])c5[2H])n4)c32)c([2H])c1[2H]]

7. The organic light emitting device according to claim 1, wherein
the compound of Chemical Formulas 2 is represented by one of the following Chemical Formulas 2-1 to 2-3: 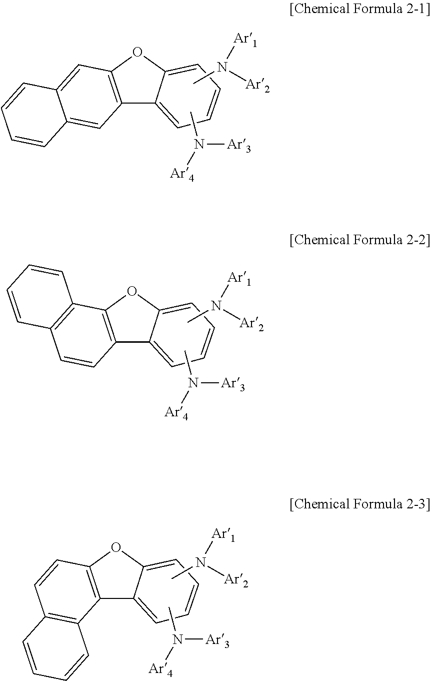
in Chemical Formulas 2-1 to 2-3,
Ar′₁ to Ar′₄ are the same as defined in claim 1.

8. The organic light emitting device according to claim 1, wherein
Ar′₁ to Ar′₄ are each independently phenyl; biphenylyl; terphenylyl; naphthyl; phenylnaphthyl; naphthylphenyl; naphthylbiphenylyl; phenylnaphthylphenyl; phenylterphenylyl; phenanthrenyl; dibenzofuranyl; or dibenzothiophenyl.

9. The organic light emitting device according to claim 1, wherein
the compound represented by Chemical Formula 2 is one selected from the following: 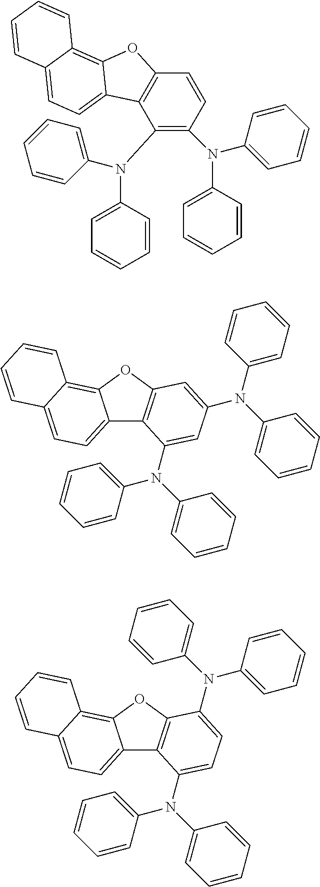 [SMILES: c1ccc(N(c2ccccc2)c2ccc3oc4c5ccccc5ccc4c3c2N(c2ccccc2)c2ccccc2)cc1 ; c1ccc(N(c2ccccc2)c2cc(N(c3ccccc3)c3ccccc3)c3c(c2)oc2c4ccccc4ccc23)cc1 ; c1ccc(N(c2ccccc2)c2ccc(N(c3ccccc3)c3ccccc3)c3c2oc2c4ccccc4ccc23)cc1] 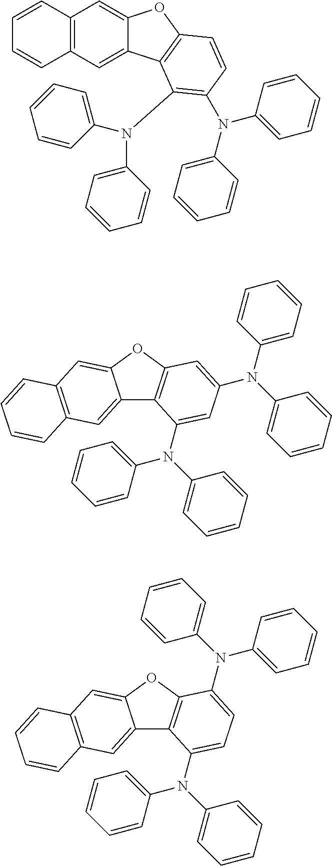 [SMILES: c1ccc(N(c2ccccc2)c2ccc3oc4cc5ccccc5cc4c3c2N(c2ccccc2)c2ccccc2)cc1 ; c1ccc(N(c2ccccc2)c2cc(N(c3ccccc3)c3ccccc3)c3c(c2)oc2cc4ccccc4cc23)cc1 ; c1ccc(N(c2ccccc2)c2ccc(N(c3ccccc3)c3ccccc3)c3c2oc2cc4ccccc4cc23)cc1] 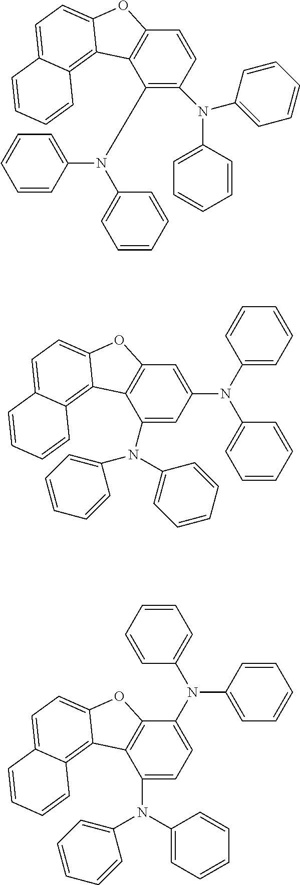 [SMILES: c1ccc(N(c2ccccc2)c2ccc3oc4ccc5ccccc5c4c3c2N(c2ccccc2)c2ccccc2)cc1 ; c1ccc(N(c2ccccc2)c2cc(N(c3ccccc3)c3ccccc3)c3c(c2)oc2ccc4ccccc4c23)cc1 ; c1ccc(N(c2ccccc2)c2ccc(N(c3ccccc3)c3ccccc3)c3c2oc2ccc4ccccc4c23)cc1] 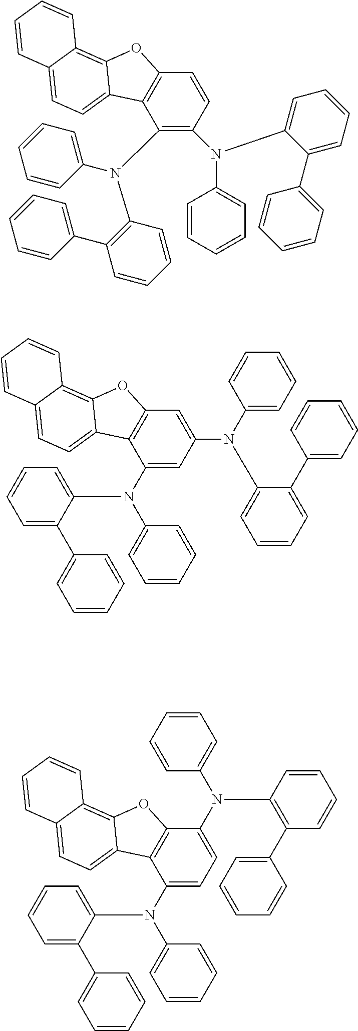 [SMILES: c1ccc(-c2ccccc2N(c2ccccc2)c2ccc3oc4c5ccccc5ccc4c3c2N(c2ccccc2)c2ccccc2-c2ccccc2)cc1 ; c1ccc(-c2ccccc2N(c2ccccc2)c2cc(N(c3ccccc3)c3ccccc3-c3ccccc3)c3c(c2)oc2c4ccccc4ccc23)cc1 ; c1ccc(-c2ccccc2N(c2ccccc2)c2ccc(N(c3ccccc3)c3ccccc3-c3ccccc3)c3c2oc2c4ccccc4ccc23)cc1] 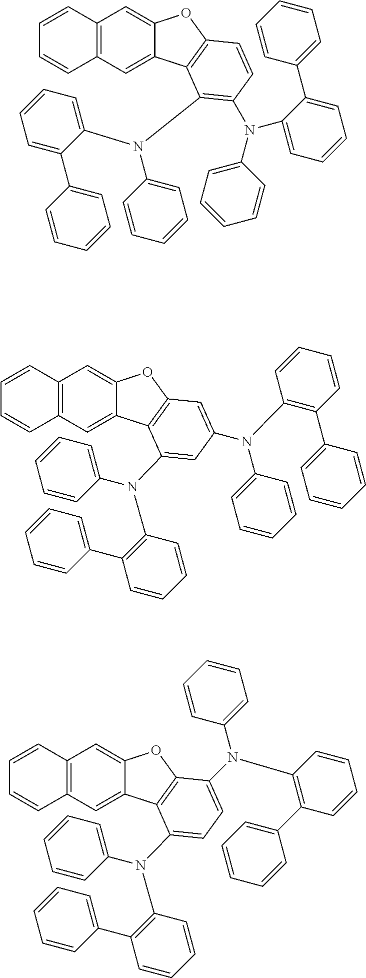 [SMILES: c1ccc(-c2ccccc2N(c2ccccc2)c2ccc3oc4cc5ccccc5cc4c3c2N(c2ccccc2)c2ccccc2-c2ccccc2)cc1 ; c1ccc(-c2ccccc2N(c2ccccc2)c2cc(N(c3ccccc3)c3ccccc3-c3ccccc3)c3c(c2)oc2cc4ccccc4cc23)cc1 ; c1ccc(-c2ccccc2N(c2ccccc2)c2ccc(N(c3ccccc3)c3ccccc3-c3ccccc3)c3c2oc2cc4ccccc4cc23)cc1] 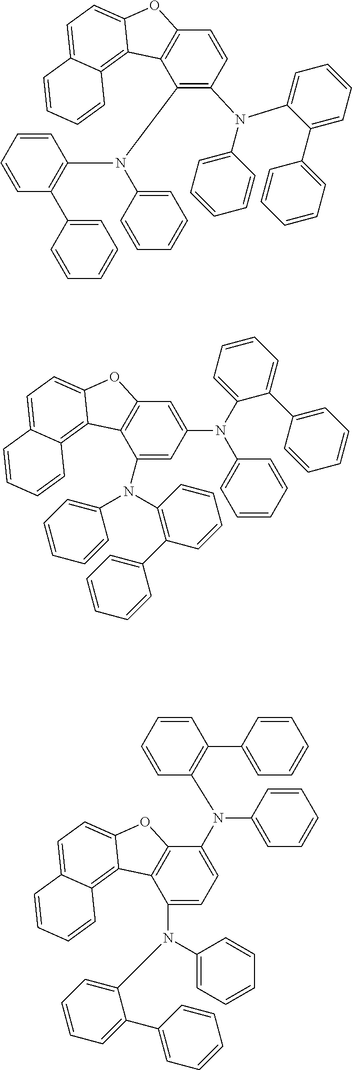 [SMILES: c1ccc(-c2ccccc2N(c2ccccc2)c2ccc3oc4ccc5ccccc5c4c3c2N(c2ccccc2)c2ccccc2-c2ccccc2)cc1 ; c1ccc(-c2ccccc2N(c2ccccc2)c2cc(N(c3ccccc3)c3ccccc3-c3ccccc3)c3c(c2)oc2ccc4ccccc4c23)cc1 ; c1ccc(-c2ccccc2N(c2ccccc2)c2ccc(N(c3ccccc3)c3ccccc3-c3ccccc3)c3c2oc2ccc4ccccc4c23)cc1] 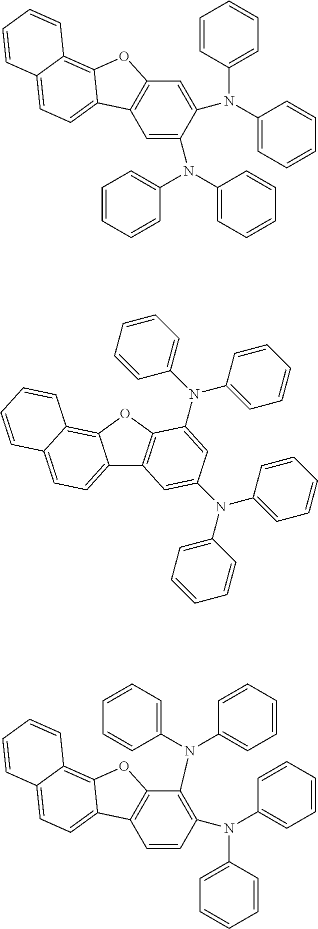 [SMILES: c1ccc(N(c2ccccc2)c2cc3oc4c5ccccc5ccc4c3cc2N(c2ccccc2)c2ccccc2)cc1 ; c1ccc(N(c2ccccc2)c2cc(N(c3ccccc3)c3ccccc3)c3oc4c5ccccc5ccc4c3c2)cc1 ; c1ccc(N(c2ccccc2)c2ccc3c(oc4c5ccccc5ccc34)c2N(c2ccccc2)c2ccccc2)cc1] 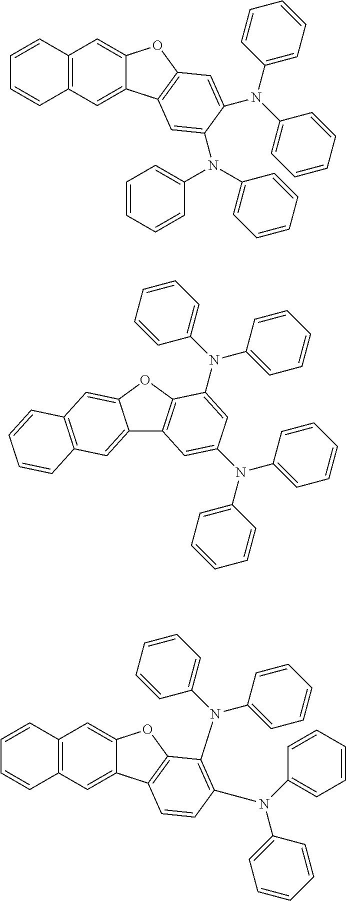 [SMILES: c1ccc(N(c2ccccc2)c2cc3oc4cc5ccccc5cc4c3cc2N(c2ccccc2)c2ccccc2)cc1 ; c1ccc(N(c2ccccc2)c2cc(N(c3ccccc3)c3ccccc3)c3oc4cc5ccccc5cc4c3c2)cc1 ; c1ccc(N(c2ccccc2)c2ccc3c(oc4cc5ccccc5cc43)c2N(c2ccccc2)c2ccccc2)cc1] 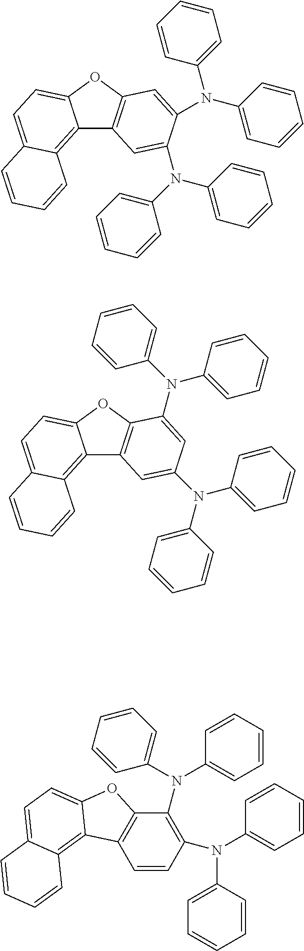 [SMILES: c1ccc(N(c2ccccc2)c2cc3oc4ccc5ccccc5c4c3cc2N(c2ccccc2)c2ccccc2)cc1 ; c1ccc(N(c2ccccc2)c2cc(N(c3ccccc3)c3ccccc3)c3oc4ccc5ccccc5c4c3c2)cc1 ; c1ccc(N(c2ccccc2)c2ccc3c(oc4ccc5ccccc5c43)c2N(c2ccccc2)c2ccccc2)cc1] 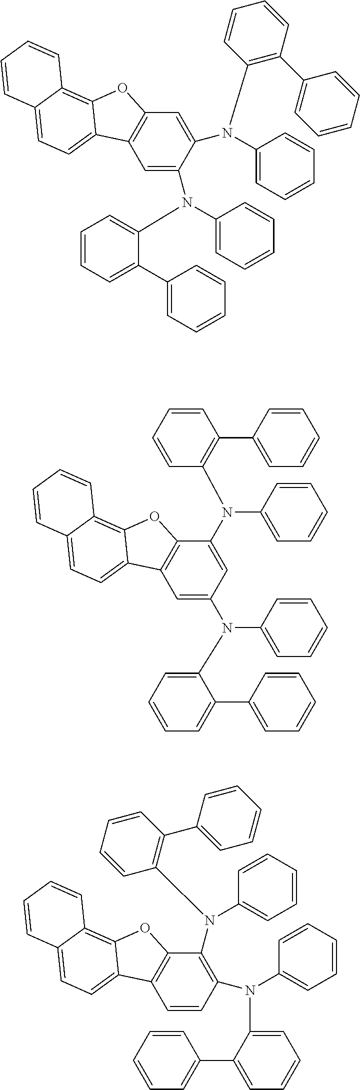 [SMILES: c1ccc(-c2ccccc2N(c2ccccc2)c2cc3oc4c5ccccc5ccc4c3cc2N(c2ccccc2)c2ccccc2-c2ccccc2)cc1 ; c1ccc(-c2ccccc2N(c2ccccc2)c2cc(N(c3ccccc3)c3ccccc3-c3ccccc3)c3oc4c5ccccc5ccc4c3c2)cc1 ; c1ccc(-c2ccccc2N(c2ccccc2)c2ccc3c(oc4c5ccccc5ccc34)c2N(c2ccccc2)c2ccccc2-c2ccccc2)cc1] 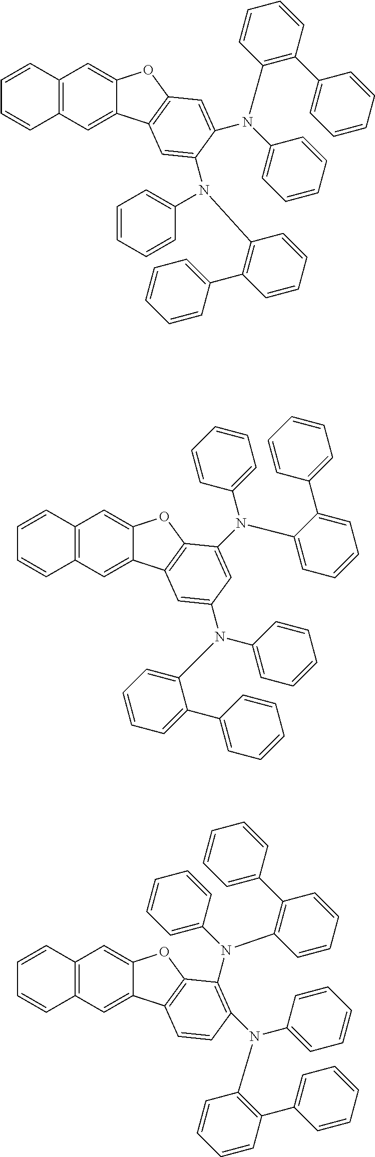 [SMILES: c1ccc(-c2ccccc2N(c2ccccc2)c2cc3oc4cc5ccccc5cc4c3cc2N(c2ccccc2)c2ccccc2-c2ccccc2)cc1 ; c1ccc(-c2ccccc2N(c2ccccc2)c2cc(N(c3ccccc3)c3ccccc3-c3ccccc3)c3oc4cc5ccccc5cc4c3c2)cc1 ; c1ccc(-c2ccccc2N(c2ccccc2)c2ccc3c(oc4cc5ccccc5cc43)c2N(c2ccccc2)c2ccccc2-c2ccccc2)cc1] 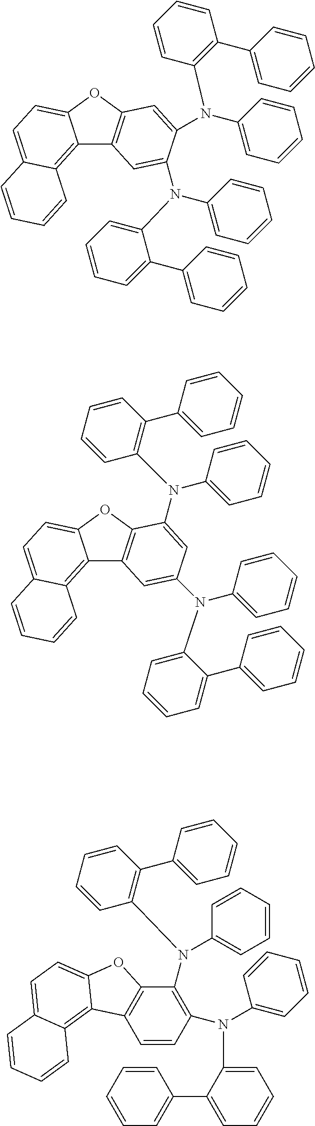 [SMILES: c1ccc(-c2ccccc2N(c2ccccc2)c2cc3oc4ccc5ccccc5c4c3cc2N(c2ccccc2)c2ccccc2-c2ccccc2)cc1 ; c1ccc(-c2ccccc2N(c2ccccc2)c2cc(N(c3ccccc3)c3ccccc3-c3ccccc3)c3oc4ccc5ccccc5c4c3c2)cc1 ; c1ccc(-c2ccccc2N(c2ccccc2)c2ccc3c(oc4ccc5ccccc5c43)c2N(c2ccccc2)c2ccccc2-c2ccccc2)cc1] 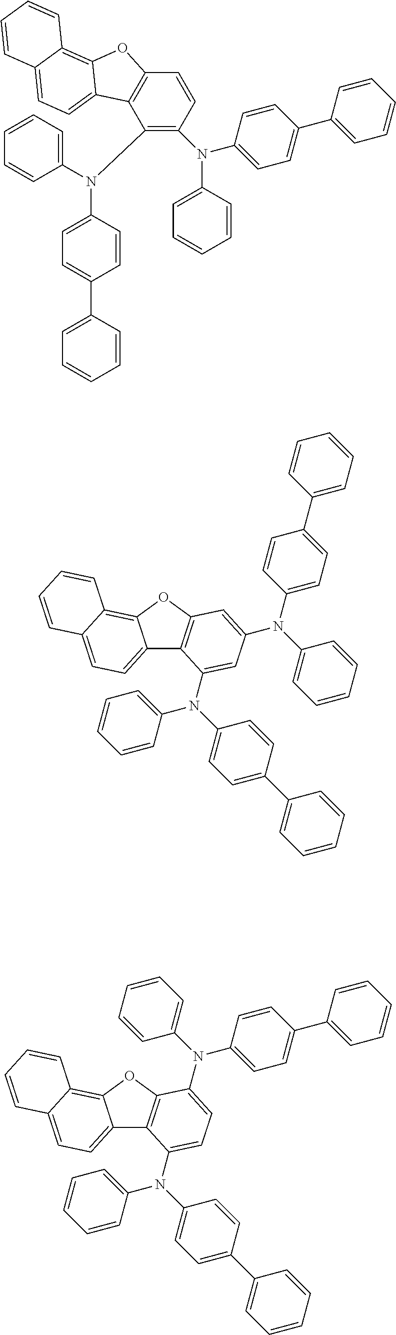 [SMILES: c1ccc(-c2ccc(N(c3ccccc3)c3ccc4oc5c6ccccc6ccc5c4c3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4c(c3)oc3c5ccccc5ccc34)cc2)cc1 ; c1ccc(-c2ccc(N(c3ccccc3)c3ccc(N(c4ccccc4)c4ccc(-c5ccccc5)cc4)c4c3oc3c5ccccc5ccc34)cc2)cc1] 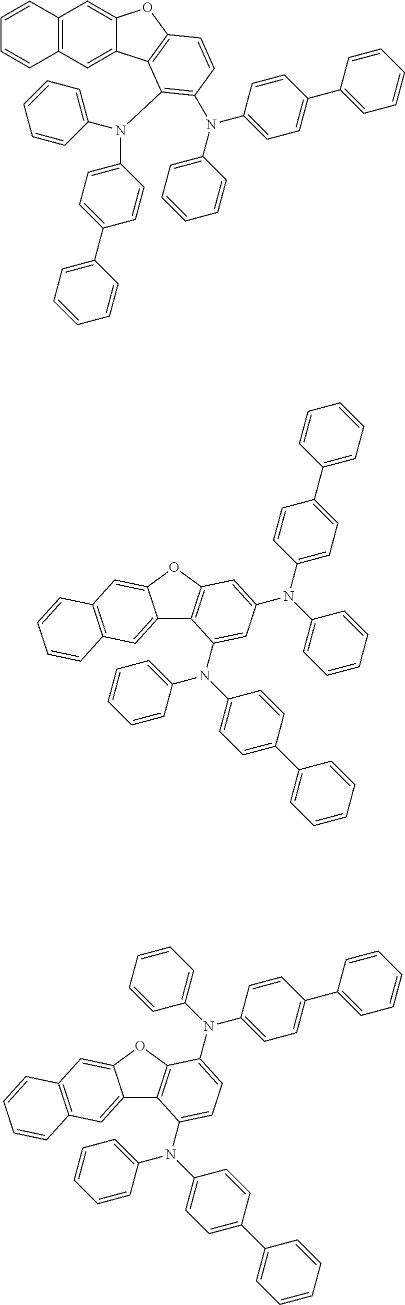 [SMILES: c1ccc(-c2ccc(N(c3ccccc3)c3ccc4oc5cc6ccccc6cc5c4c3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4c(c3)oc3cc5ccccc5cc34)cc2)cc1 ; c1ccc(-c2ccc(N(c3ccccc3)c3ccc(N(c4ccccc4)c4ccc(-c5ccccc5)cc4)c4c3oc3cc5ccccc5cc34)cc2)cc1] 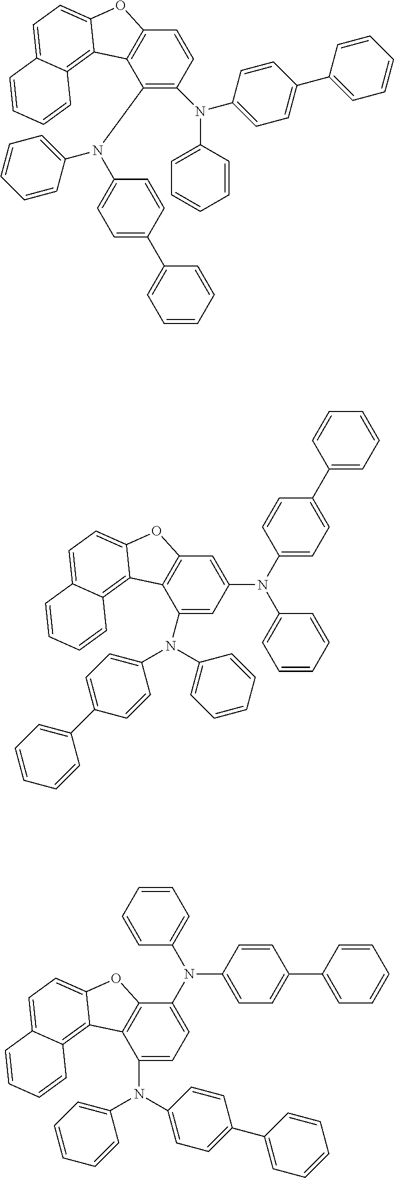 [SMILES: c1ccc(-c2ccc(N(c3ccccc3)c3ccc4oc5ccc6ccccc6c5c4c3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4c(c3)oc3ccc5ccccc5c34)cc2)cc1 ; c1ccc(-c2ccc(N(c3ccccc3)c3ccc(N(c4ccccc4)c4ccc(-c5ccccc5)cc4)c4c3oc3ccc5ccccc5c34)cc2)cc1] 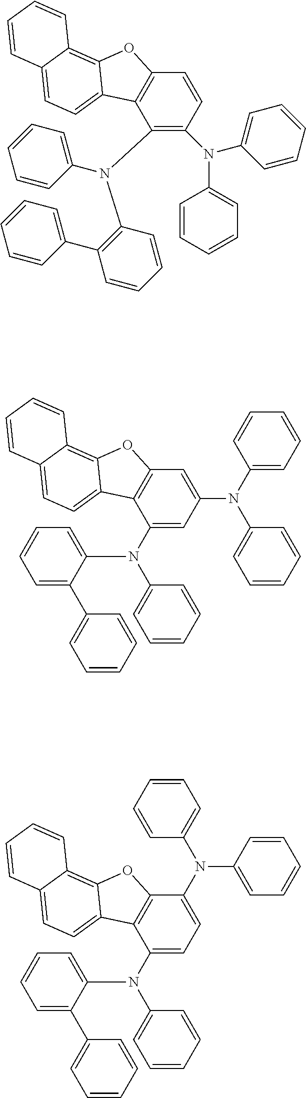 [SMILES: c1ccc(-c2ccccc2N(c2ccccc2)c2c(N(c3ccccc3)c3ccccc3)ccc3oc4c5ccccc5ccc4c23)cc1 ; c1ccc(-c2ccccc2N(c2ccccc2)c2cc(N(c3ccccc3)c3ccccc3)cc3oc4c5ccccc5ccc4c23)cc1 ; c1ccc(-c2ccccc2N(c2ccccc2)c2ccc(N(c3ccccc3)c3ccccc3)c3oc4c5ccccc5ccc4c23)cc1] 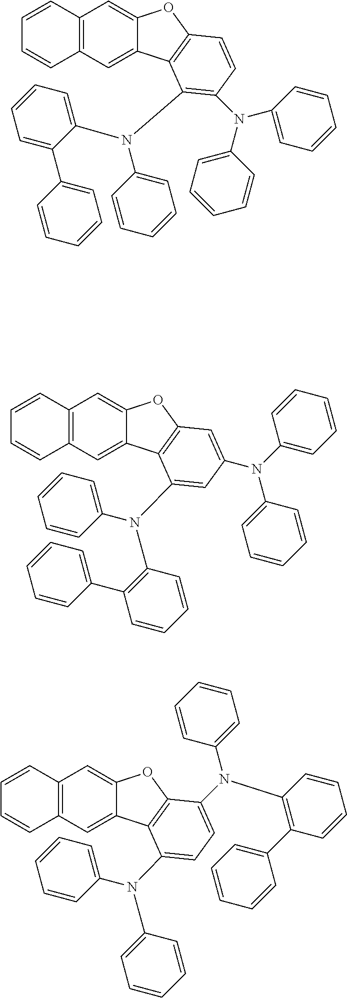 [SMILES: c1ccc(-c2ccccc2N(c2ccccc2)c2c(N(c3ccccc3)c3ccccc3)ccc3oc4cc5ccccc5cc4c23)cc1 ; c1ccc(-c2ccccc2N(c2ccccc2)c2cc(N(c3ccccc3)c3ccccc3)cc3oc4cc5ccccc5cc4c23)cc1 ; c1ccc(-c2ccccc2N(c2ccccc2)c2ccc(N(c3ccccc3)c3ccccc3)c3c2oc2cc4ccccc4cc23)cc1] 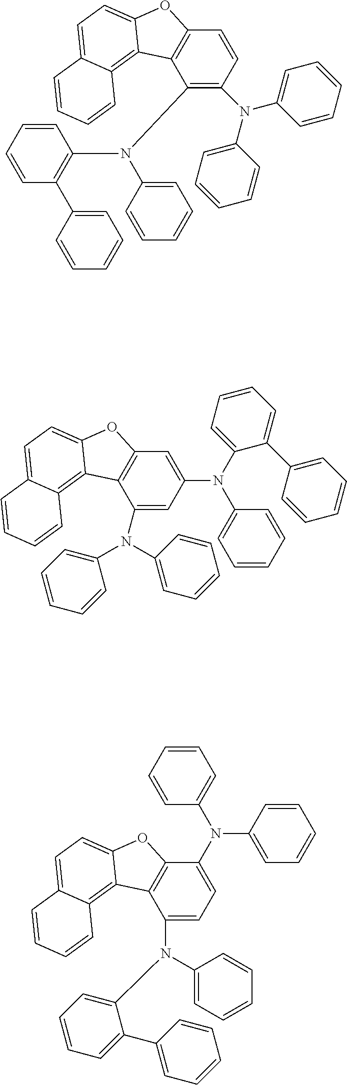 [SMILES: c1ccc(-c2ccccc2N(c2ccccc2)c2c(N(c3ccccc3)c3ccccc3)ccc3oc4ccc5ccccc5c4c23)cc1 ; c1ccc(-c2ccccc2N(c2ccccc2)c2cc(N(c3ccccc3)c3ccccc3)c3c(c2)oc2ccc4ccccc4c23)cc1 ; c1ccc(-c2ccccc2N(c2ccccc2)c2ccc(N(c3ccccc3)c3ccccc3)c3oc4ccc5ccccc5c4c23)cc1] 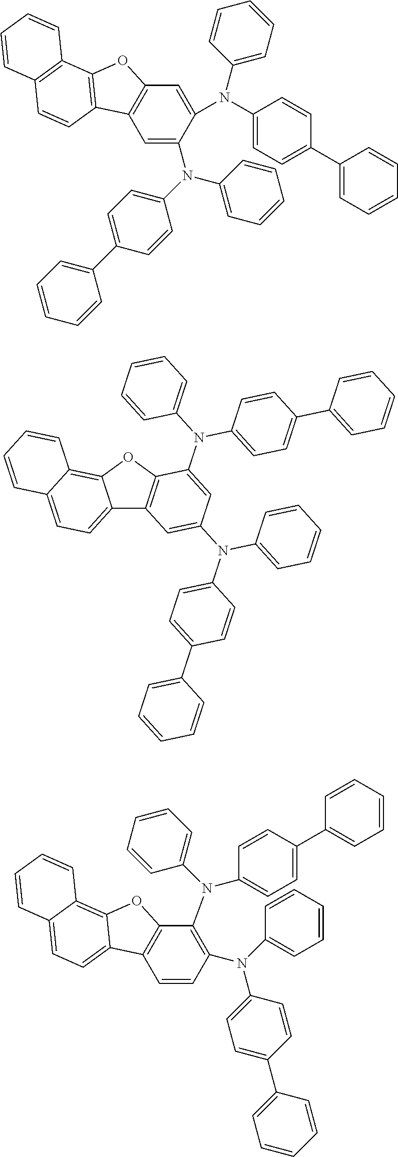 [SMILES: c1ccc(-c2ccc(N(c3ccccc3)c3cc4oc5c6ccccc6ccc5c4cc3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4oc5c6ccccc6ccc5c4c3)cc2)cc1 ; c1ccc(-c2ccc(N(c3ccccc3)c3ccc4c(oc5c6ccccc6ccc45)c3N(c3ccccc3)c3ccc(-c4ccccc4)cc3)cc2)cc1] 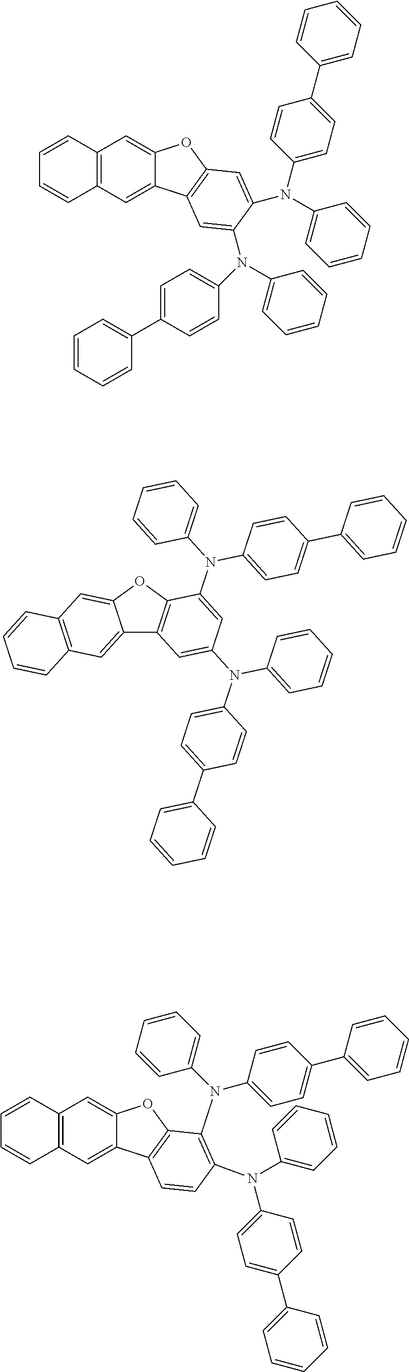 [SMILES: c1ccc(-c2ccc(N(c3ccccc3)c3cc4oc5cc6ccccc6cc5c4cc3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4oc5cc6ccccc6cc5c4c3)cc2)cc1 ; c1ccc(-c2ccc(N(c3ccccc3)c3ccc4c(oc5cc6ccccc6cc54)c3N(c3ccccc3)c3ccc(-c4ccccc4)cc3)cc2)cc1] 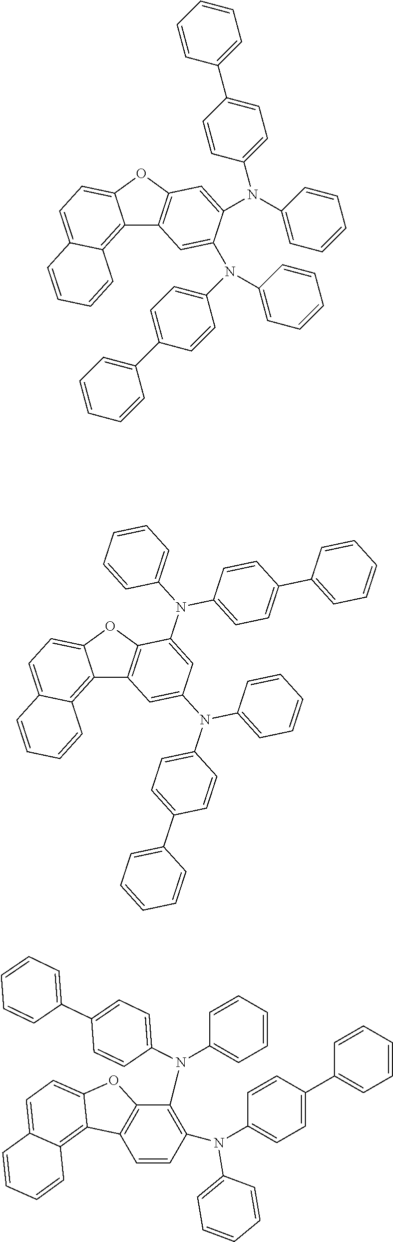 [SMILES: c1ccc(-c2ccc(N(c3ccccc3)c3cc4oc5ccc6ccccc6c5c4cc3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4oc5ccc6ccccc6c5c4c3)cc2)cc1 ; c1ccc(-c2ccc(N(c3ccccc3)c3ccc4c(oc5ccc6ccccc6c54)c3N(c3ccccc3)c3ccc(-c4ccccc4)cc3)cc2)cc1] 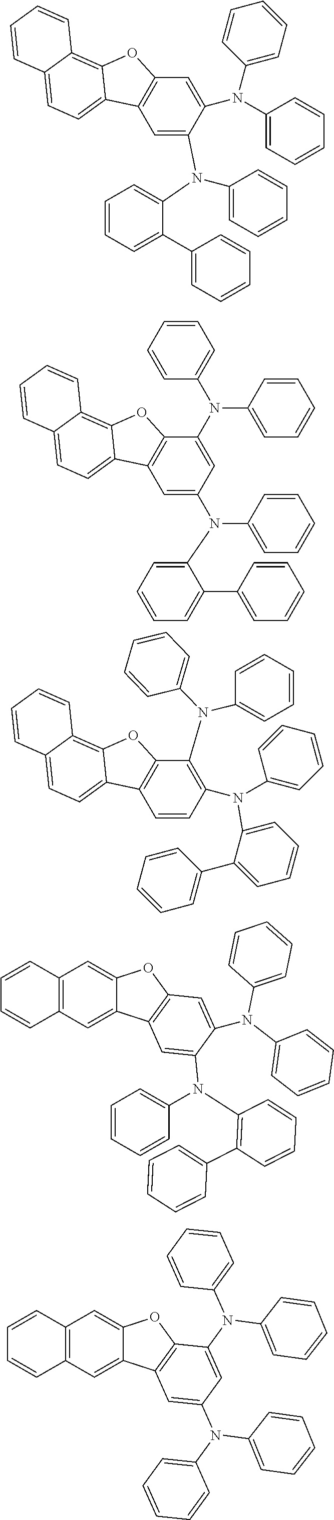 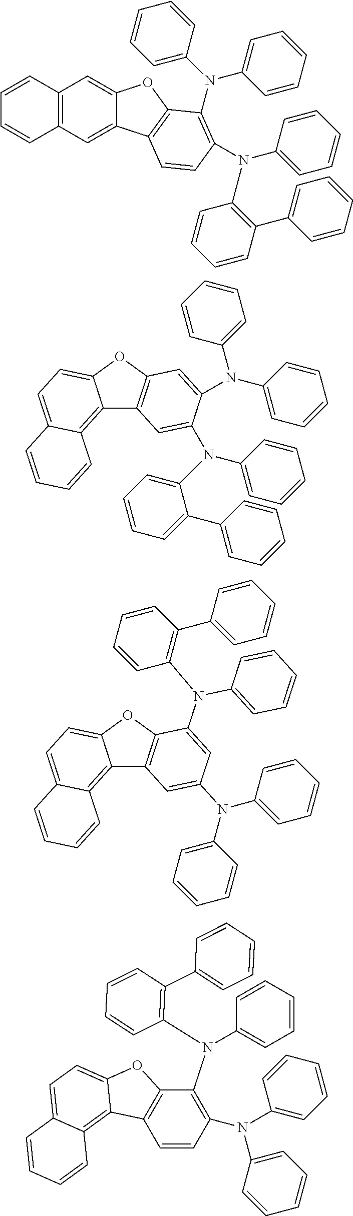 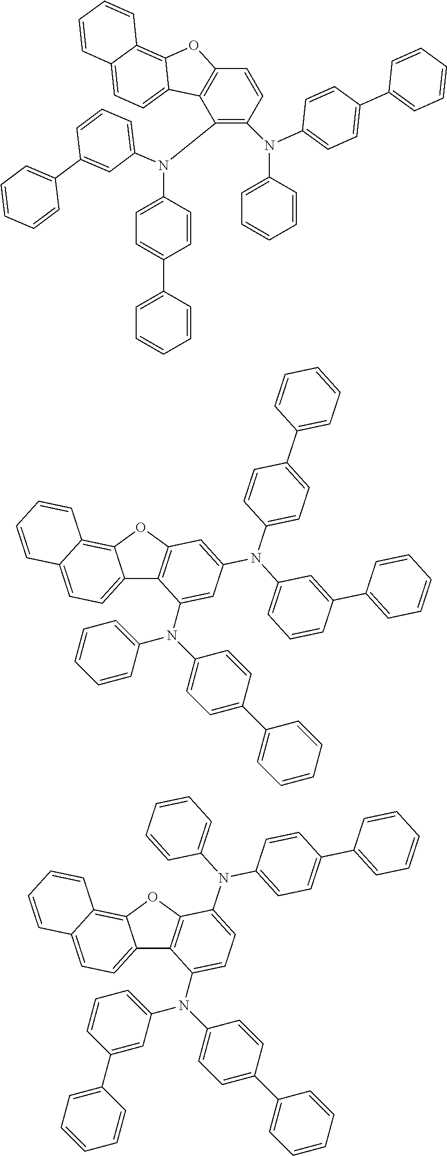 [SMILES: c1ccc(-c2ccc(N(c3ccccc3)c3ccc4oc5c6ccccc6ccc5c4c3N(c3ccc(-c4ccccc4)cc3)c3cccc(-c4ccccc4)c3)cc2)cc1 ; c1ccc(-c2ccc(N(c3cccc(-c4ccccc4)c3)c3cc(N(c4ccccc4)c4ccc(-c5ccccc5)cc4)c4c(c3)oc3c5ccccc5ccc34)cc2)cc1 ; c1ccc(-c2ccc(N(c3ccccc3)c3ccc(N(c4ccc(-c5ccccc5)cc4)c4cccc(-c5ccccc5)c4)c4c3oc3c5ccccc5ccc34)cc2)cc1] 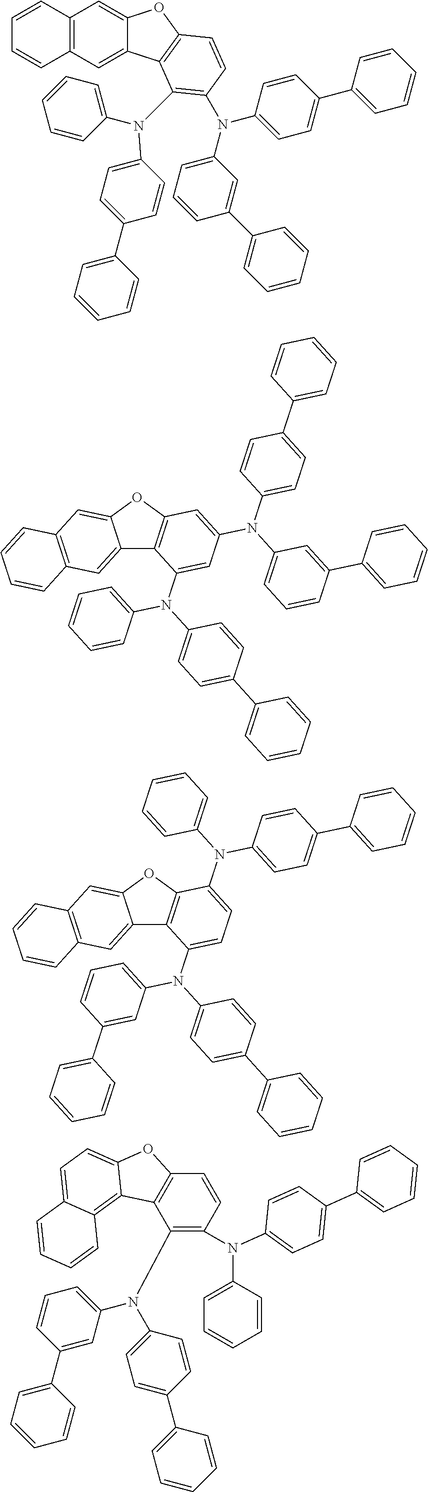 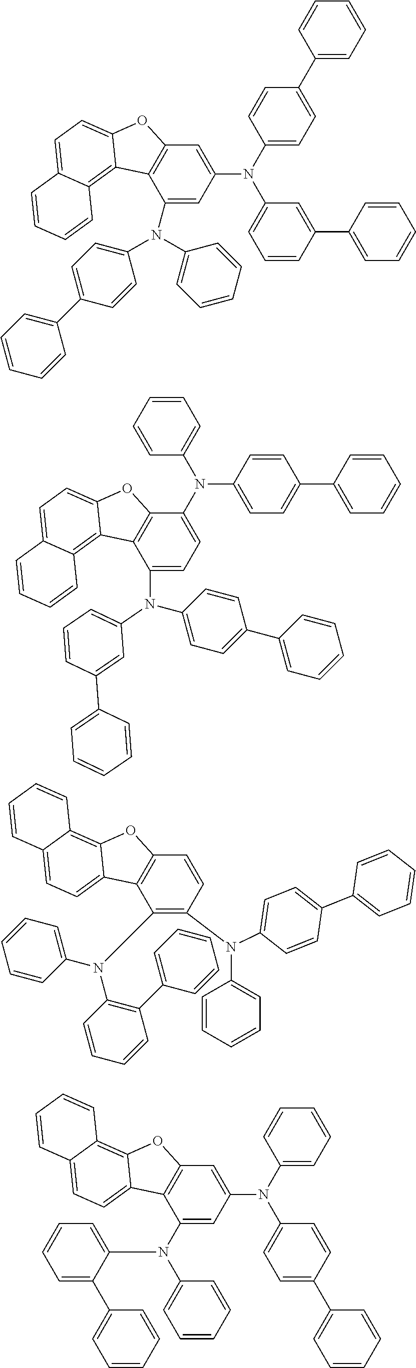 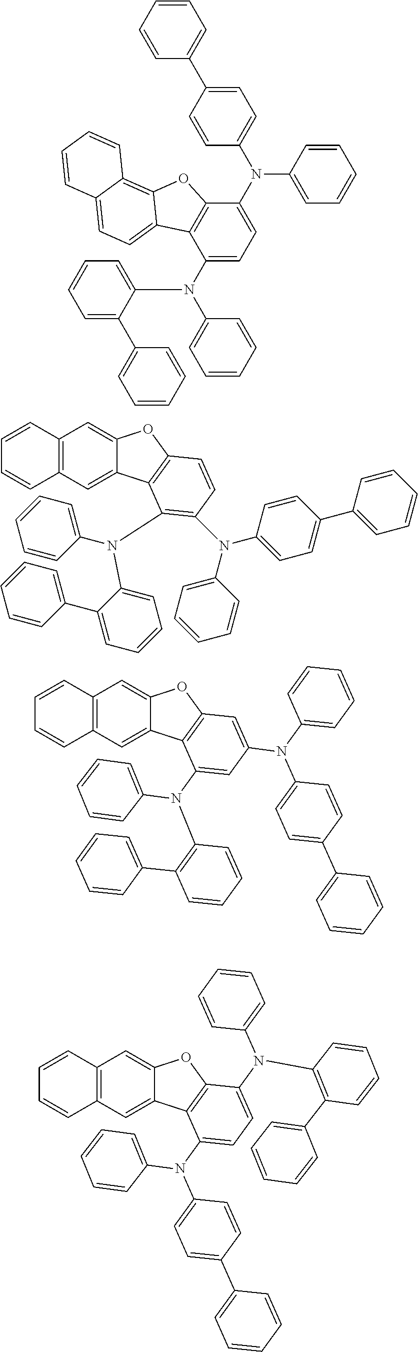 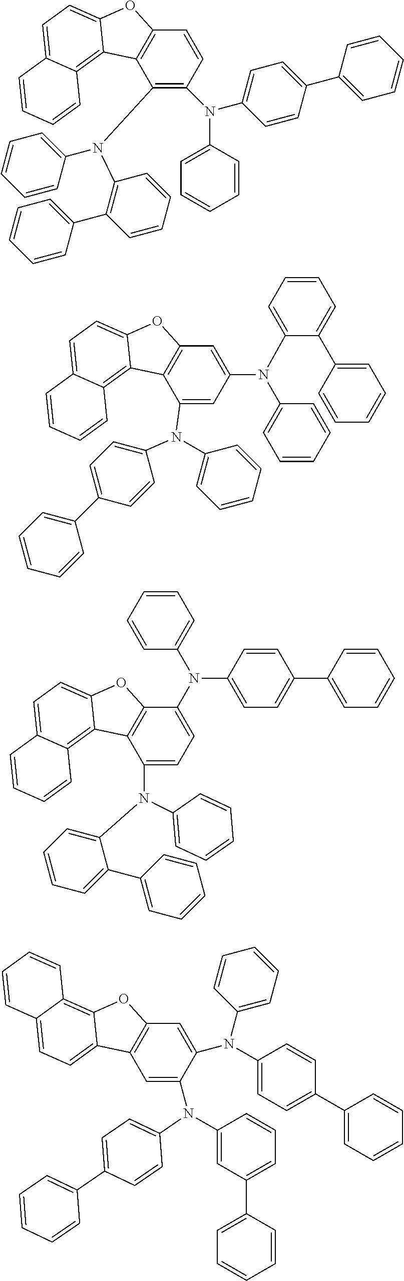 [SMILES: c1ccc(-c2ccc(N(c3ccccc3)c3ccc4oc5ccc6ccccc6c5c4c3N(c3ccccc3)c3ccccc3-c3ccccc3)cc2)cc1 ; c1ccc(-c2ccc(N(c3ccccc3)c3cc(N(c4ccccc4)c4ccccc4-c4ccccc4)cc4oc5ccc6ccccc6c5c34)cc2)cc1 ; c1ccc(-c2ccc(N(c3ccccc3)c3ccc(N(c4ccccc4)c4ccccc4-c4ccccc4)c4c3oc3ccc5ccccc5c34)cc2)cc1 ; c1ccc(-c2ccc(N(c3ccccc3)c3cc4oc5c6ccccc6ccc5c4cc3N(c3ccc(-c4ccccc4)cc3)c3cccc(-c4ccccc4)c3)cc2)cc1] 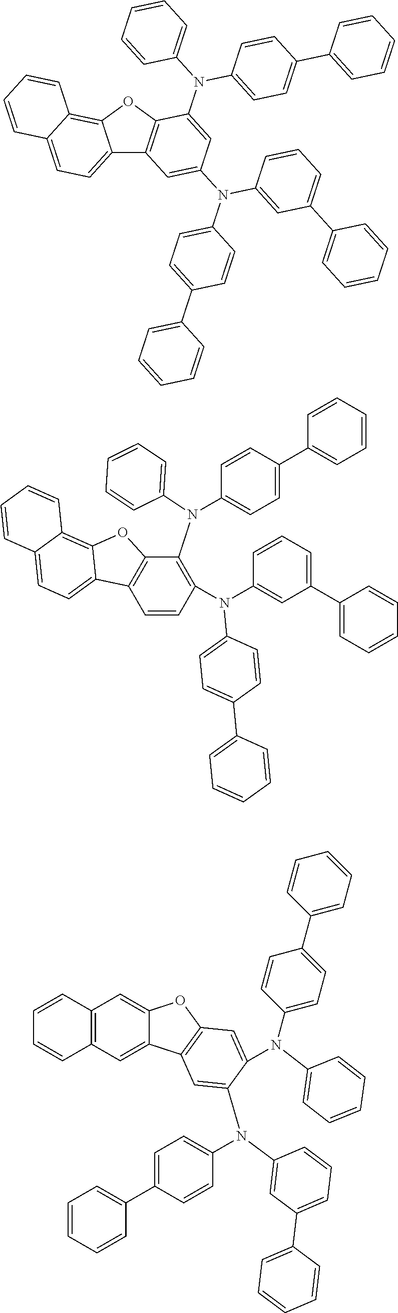 [SMILES: c1ccc(-c2ccc(N(c3cccc(-c4ccccc4)c3)c3cc(N(c4ccccc4)c4ccc(-c5ccccc5)cc4)c4oc5c6ccccc6ccc5c4c3)cc2)cc1 ; c1ccc(-c2ccc(N(c3cccc(-c4ccccc4)c3)c3ccc4c(oc5c6ccccc6ccc45)c3N(c3ccccc3)c3ccc(-c4ccccc4)cc3)cc2)cc1 ; c1ccc(-c2ccc(N(c3ccccc3)c3cc4oc5cc6ccccc6cc5c4cc3N(c3ccc(-c4ccccc4)cc3)c3cccc(-c4ccccc4)c3)cc2)cc1] 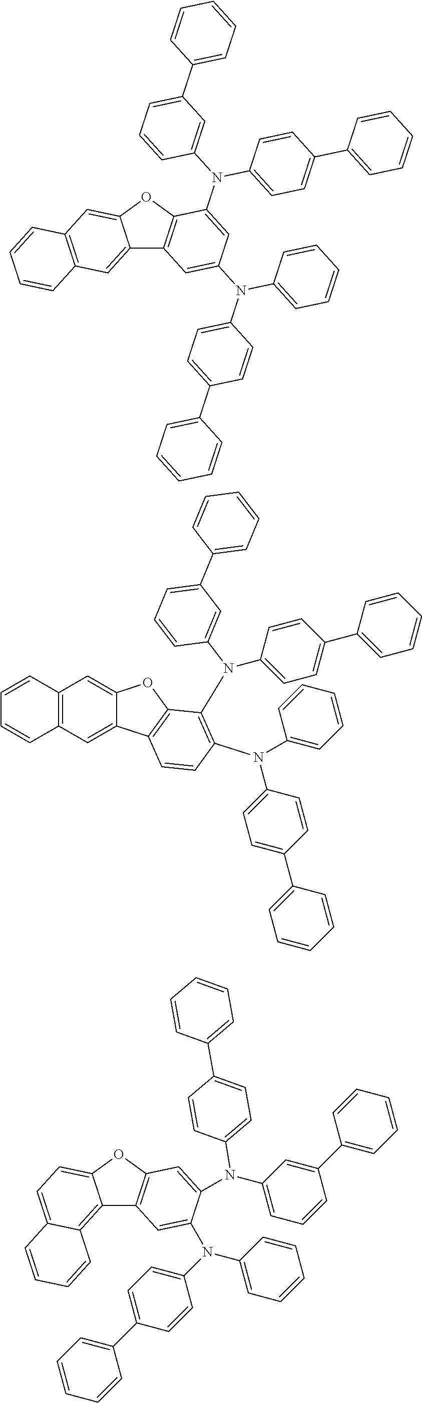 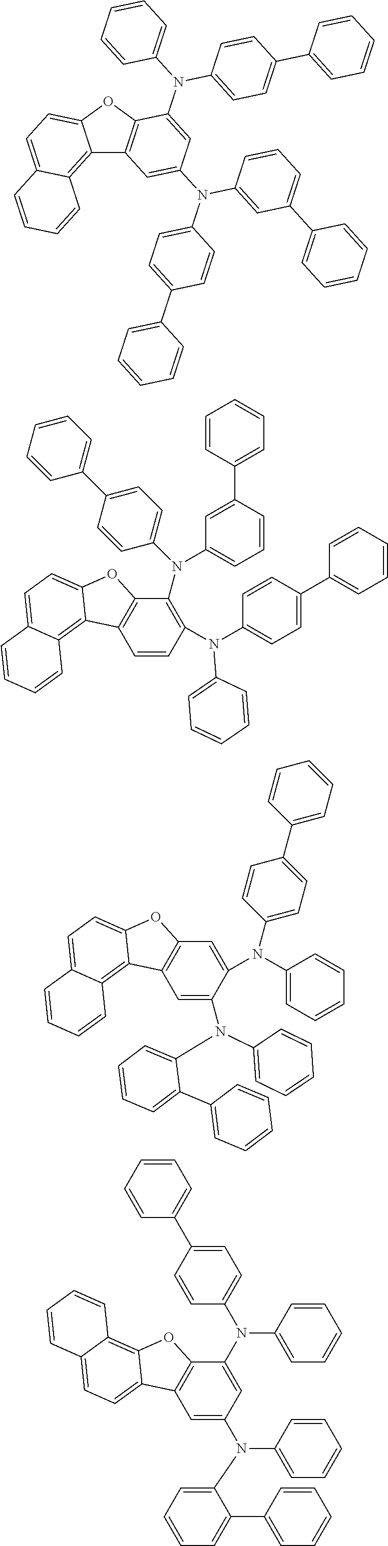 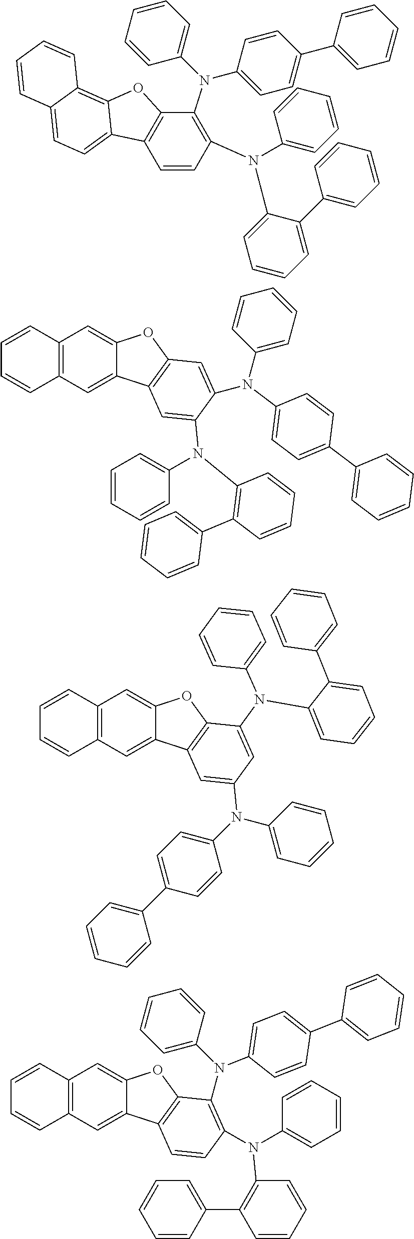 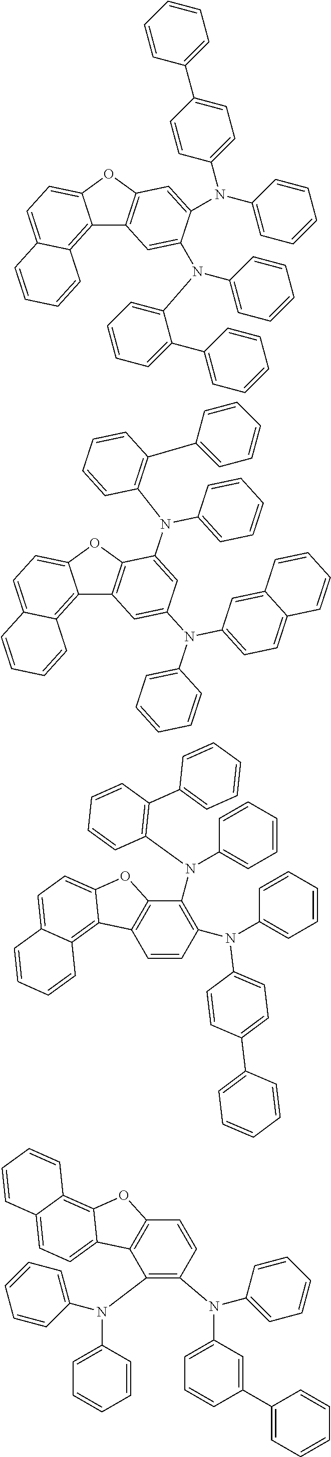 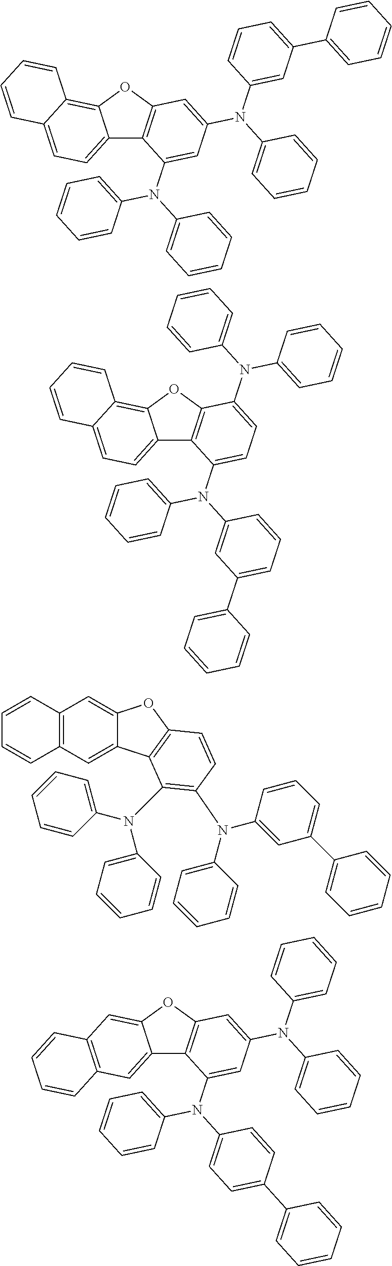 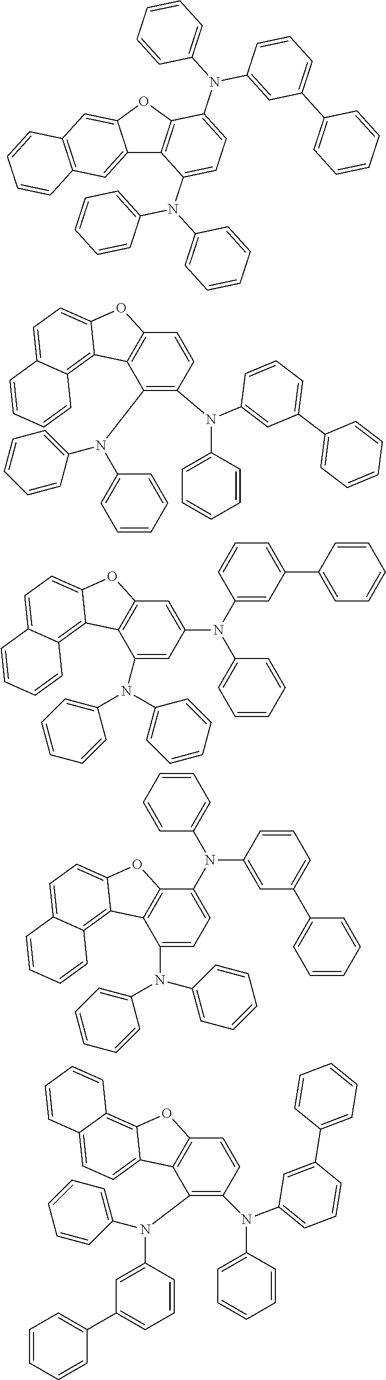 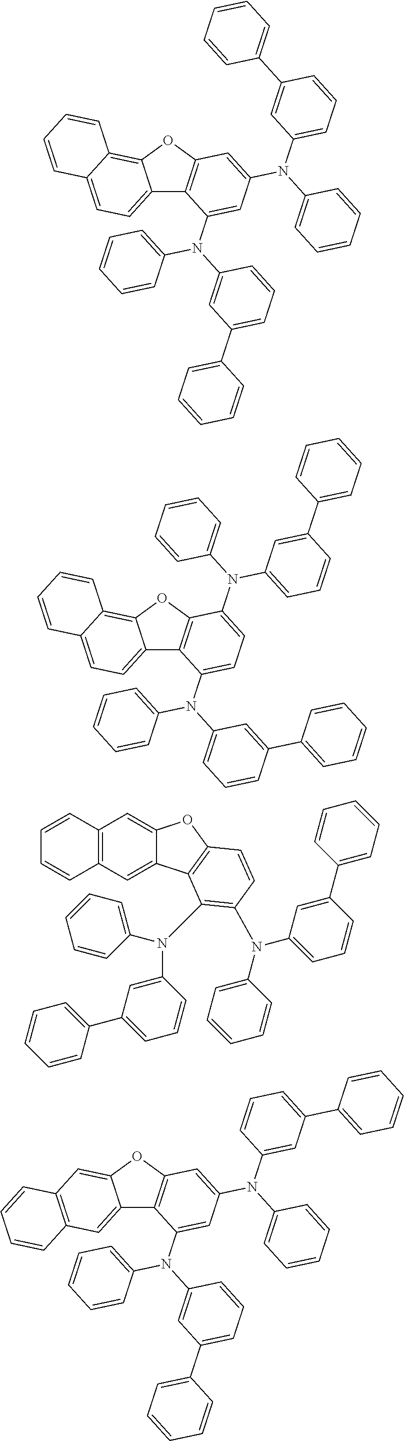 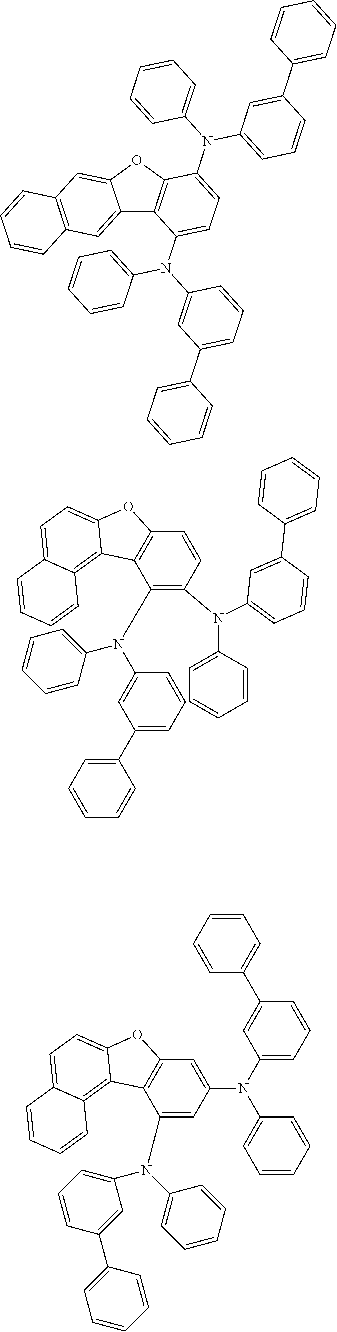 [SMILES: c1ccc(-c2cccc(N(c3ccccc3)c3ccc(N(c4ccccc4)c4cccc(-c5ccccc5)c4)c4c3oc3cc5ccccc5cc34)c2)cc1 ; c1ccc(-c2cccc(N(c3ccccc3)c3ccc4oc5ccc6ccccc6c5c4c3N(c3ccccc3)c3cccc(-c4ccccc4)c3)c2)cc1 ; c1ccc(-c2cccc(N(c3ccccc3)c3cc(N(c4ccccc4)c4cccc(-c5ccccc5)c4)c4c(c3)oc3ccc5ccccc5c34)c2)cc1] 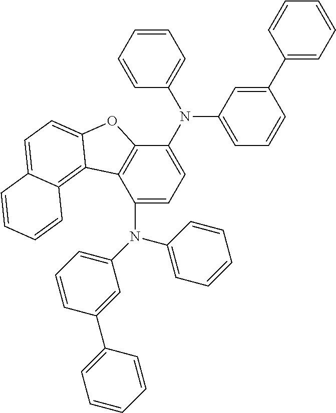 [SMILES: c1ccc(-c2cccc(N(c3ccccc3)c3ccc(N(c4ccccc4)c4cccc(-c5ccccc5)c4)c4c3oc3ccc5ccccc5c34)c2)cc1] 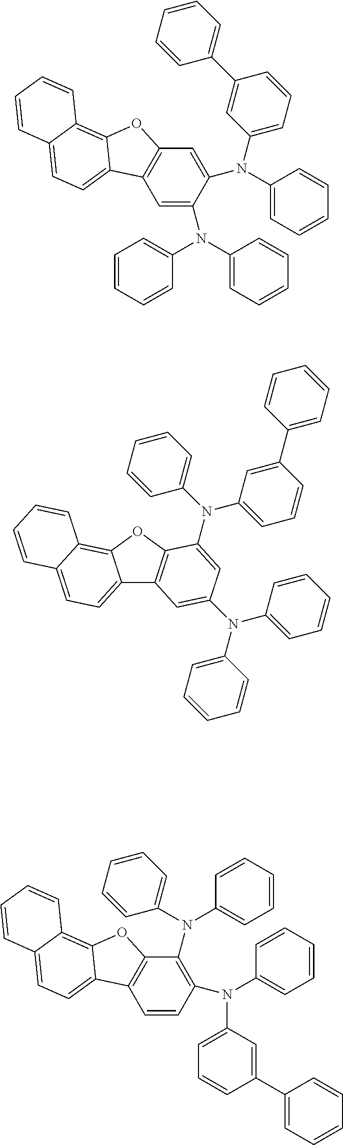 [SMILES: c1ccc(-c2cccc(N(c3ccccc3)c3cc4oc5c6ccccc6ccc5c4cc3N(c3ccccc3)c3ccccc3)c2)cc1 ; c1ccc(-c2cccc(N(c3ccccc3)c3cc(N(c4ccccc4)c4ccccc4)cc4c3oc3c5ccccc5ccc43)c2)cc1 ; c1ccc(-c2cccc(N(c3ccccc3)c3ccc4c(oc5c6ccccc6ccc45)c3N(c3ccccc3)c3ccccc3)c2)cc1] 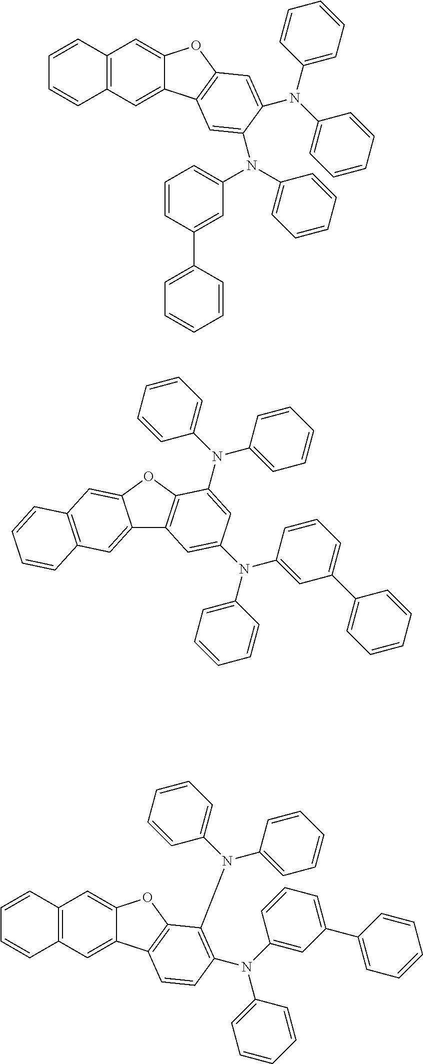 [SMILES: c1ccc(-c2cccc(N(c3ccccc3)c3cc4c(cc3N(c3ccccc3)c3ccccc3)oc3cc5ccccc5cc34)c2)cc1 ; c1ccc(-c2cccc(N(c3ccccc3)c3cc(N(c4ccccc4)c4ccccc4)c4oc5cc6ccccc6cc5c4c3)c2)cc1 ; c1ccc(-c2cccc(N(c3ccccc3)c3ccc4c(oc5cc6ccccc6cc54)c3N(c3ccccc3)c3ccccc3)c2)cc1] 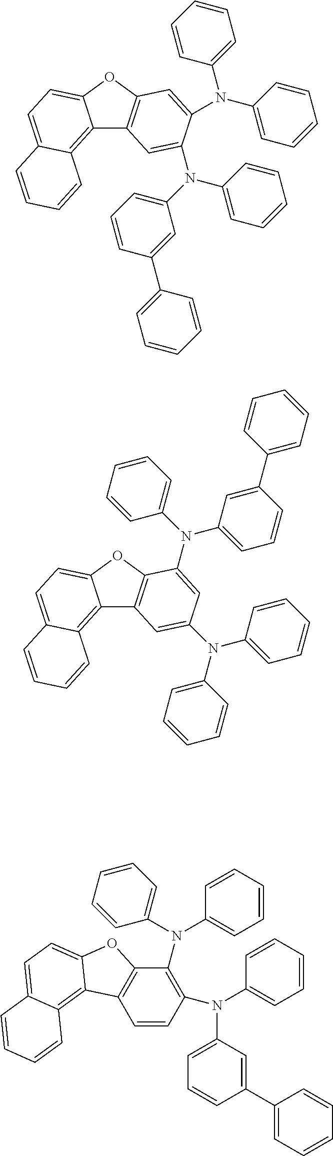 [SMILES: c1ccc(-c2cccc(N(c3ccccc3)c3cc4c(cc3N(c3ccccc3)c3ccccc3)oc3ccc5ccccc5c34)c2)cc1 ; c1ccc(-c2cccc(N(c3ccccc3)c3cc(N(c4ccccc4)c4ccccc4)cc4c3oc3ccc5ccccc5c34)c2)cc1 ; c1ccc(-c2cccc(N(c3ccccc3)c3ccc4c(oc5ccc6ccccc6c54)c3N(c3ccccc3)c3ccccc3)c2)cc1] 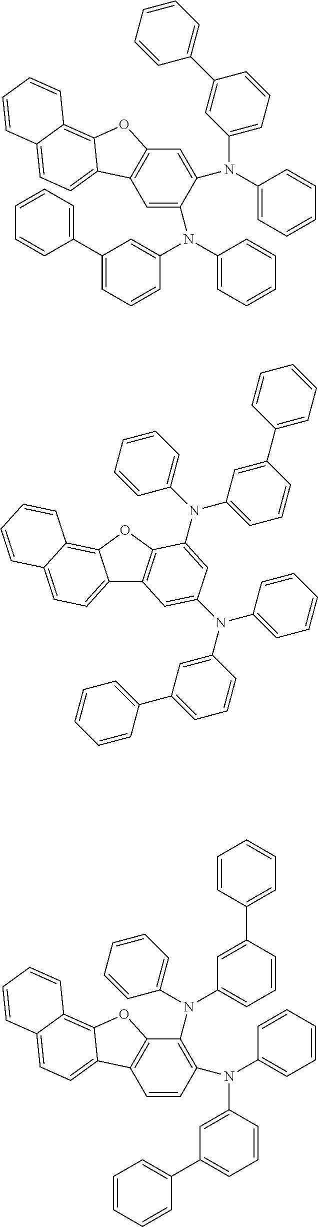 [SMILES: c1ccc(-c2cccc(N(c3ccccc3)c3cc4oc5c6ccccc6ccc5c4cc3N(c3ccccc3)c3cccc(-c4ccccc4)c3)c2)cc1 ; c1ccc(-c2cccc(N(c3ccccc3)c3cc(N(c4ccccc4)c4cccc(-c5ccccc5)c4)c4oc5c6ccccc6ccc5c4c3)c2)cc1 ; c1ccc(-c2cccc(N(c3ccccc3)c3ccc4c(oc5c6ccccc6ccc45)c3N(c3ccccc3)c3cccc(-c4ccccc4)c3)c2)cc1] 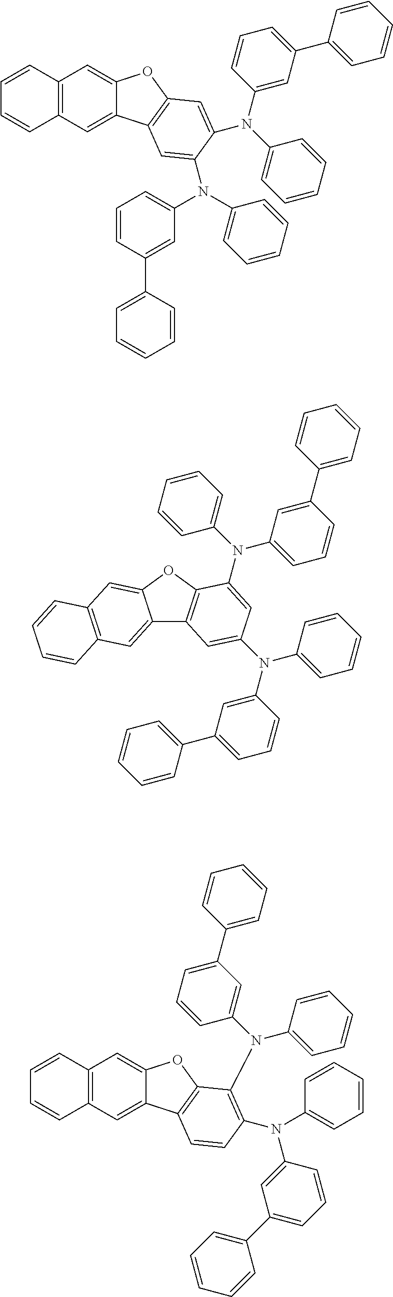 [SMILES: c1ccc(-c2cccc(N(c3ccccc3)c3cc4oc5cc6ccccc6cc5c4cc3N(c3ccccc3)c3cccc(-c4ccccc4)c3)c2)cc1 ; c1ccc(-c2cccc(N(c3ccccc3)c3cc(N(c4ccccc4)c4cccc(-c5ccccc5)c4)c4oc5cc6ccccc6cc5c4c3)c2)cc1 ; c1ccc(-c2cccc(N(c3ccccc3)c3ccc4c(oc5cc6ccccc6cc54)c3N(c3ccccc3)c3cccc(-c4ccccc4)c3)c2)cc1] 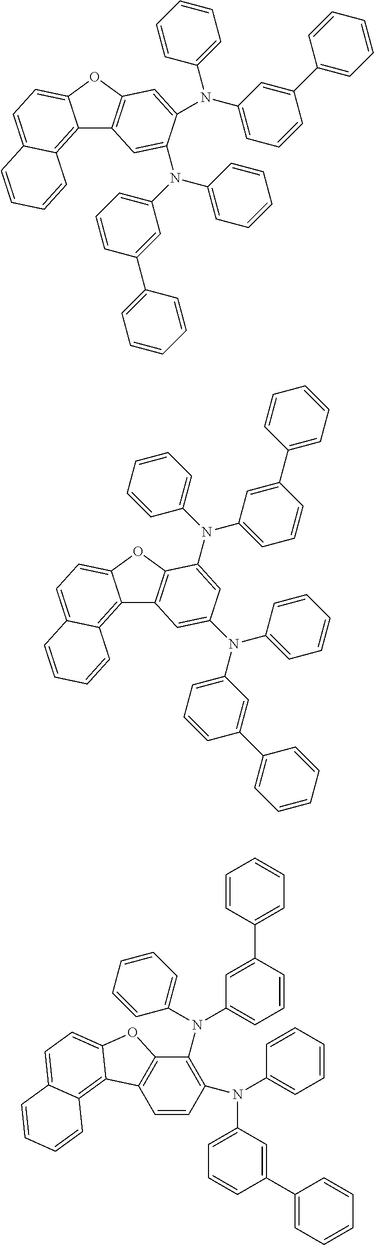 [SMILES: c1ccc(-c2cccc(N(c3ccccc3)c3cc4oc5ccc6ccccc6c5c4cc3N(c3ccccc3)c3cccc(-c4ccccc4)c3)c2)cc1 ; c1ccc(-c2cccc(N(c3ccccc3)c3cc(N(c4ccccc4)c4cccc(-c5ccccc5)c4)c4oc5ccc6ccccc6c5c4c3)c2)cc1 ; c1ccc(-c2cccc(N(c3ccccc3)c3ccc4c(oc5ccc6ccccc6c54)c3N(c3ccccc3)c3cccc(-c4ccccc4)c3)c2)cc1] 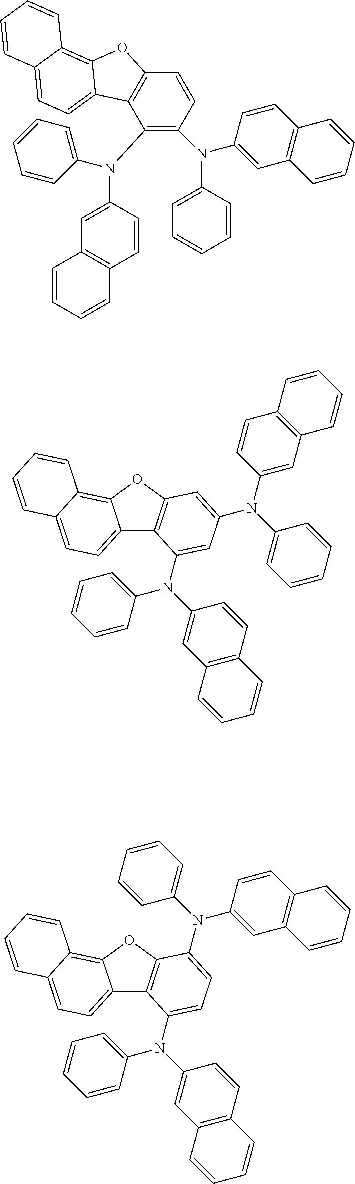 [SMILES: c1ccc(N(c2ccc3ccccc3c2)c2ccc3oc4c5ccccc5ccc4c3c2N(c2ccccc2)c2ccc3ccccc3c2)cc1 ; c1ccc(N(c2ccc3ccccc3c2)c2cc(N(c3ccccc3)c3ccc4ccccc4c3)c3c(c2)oc2c4ccccc4ccc23)cc1 ; c1ccc(N(c2ccc3ccccc3c2)c2ccc(N(c3ccccc3)c3ccc4ccccc4c3)c3c2oc2c4ccccc4ccc23)cc1] 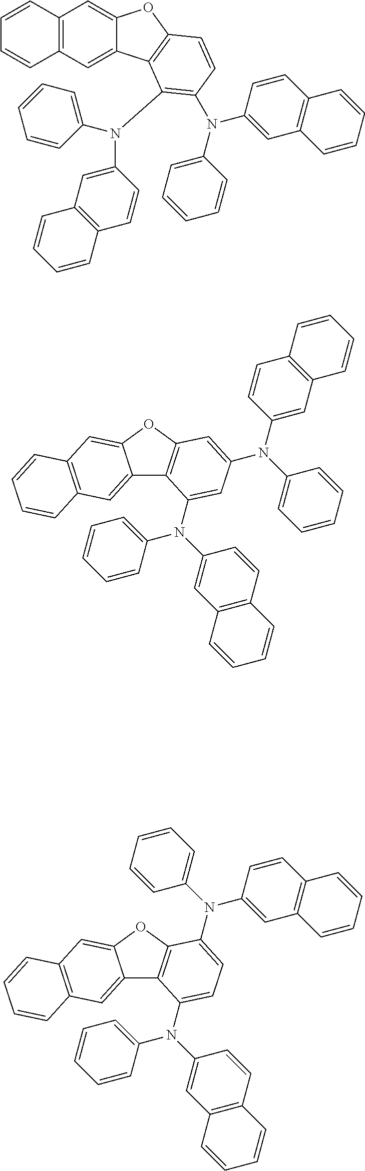 [SMILES: c1ccc(N(c2ccc3ccccc3c2)c2ccc3oc4cc5ccccc5cc4c3c2N(c2ccccc2)c2ccc3ccccc3c2)cc1 ; c1ccc(N(c2ccc3ccccc3c2)c2cc(N(c3ccccc3)c3ccc4ccccc4c3)c3c(c2)oc2cc4ccccc4cc23)cc1 ; c1ccc(N(c2ccc3ccccc3c2)c2ccc(N(c3ccccc3)c3ccc4ccccc4c3)c3c2oc2cc4ccccc4cc23)cc1] 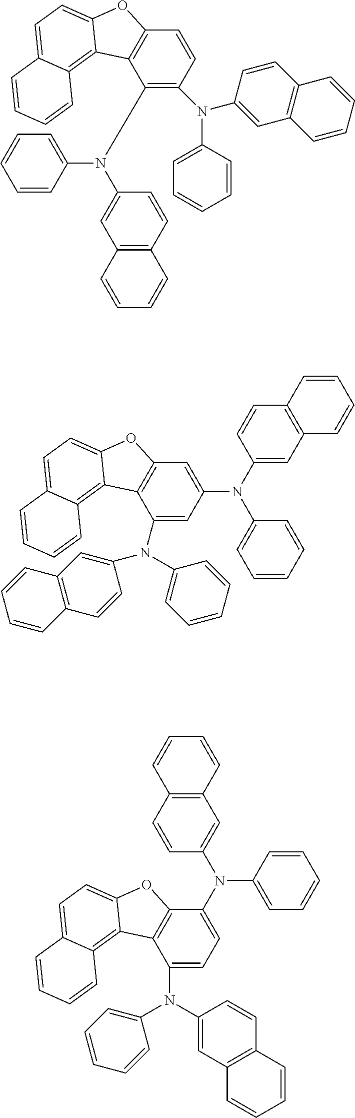 [SMILES: c1ccc(N(c2ccc3ccccc3c2)c2ccc3oc4ccc5ccccc5c4c3c2N(c2ccccc2)c2ccc3ccccc3c2)cc1 ; c1ccc(N(c2ccc3ccccc3c2)c2cc(N(c3ccccc3)c3ccc4ccccc4c3)c3c(c2)oc2ccc4ccccc4c23)cc1 ; c1ccc(N(c2ccc3ccccc3c2)c2ccc(N(c3ccccc3)c3ccc4ccccc4c3)c3c2oc2ccc4ccccc4c23)cc1] 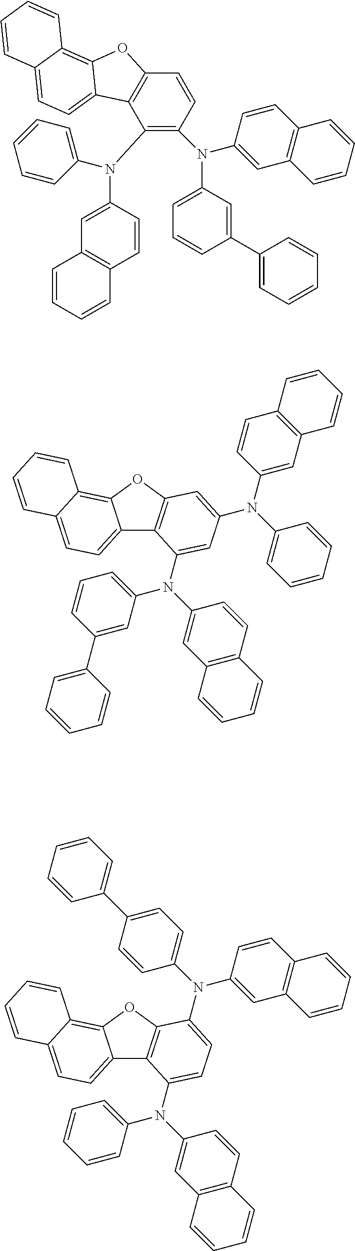 [SMILES: c1ccc(-c2cccc(N(c3ccc4ccccc4c3)c3ccc4oc5c6ccccc6ccc5c4c3N(c3ccccc3)c3ccc4ccccc4c3)c2)cc1 ; c1ccc(-c2cccc(N(c3ccc4ccccc4c3)c3cc(N(c4ccccc4)c4ccc5ccccc5c4)cc4oc5c6ccccc6ccc5c34)c2)cc1 ; c1ccc(-c2ccc(N(c3ccc4ccccc4c3)c3ccc(N(c4ccccc4)c4ccc5ccccc5c4)c4c3oc3c5ccccc5ccc34)cc2)cc1] 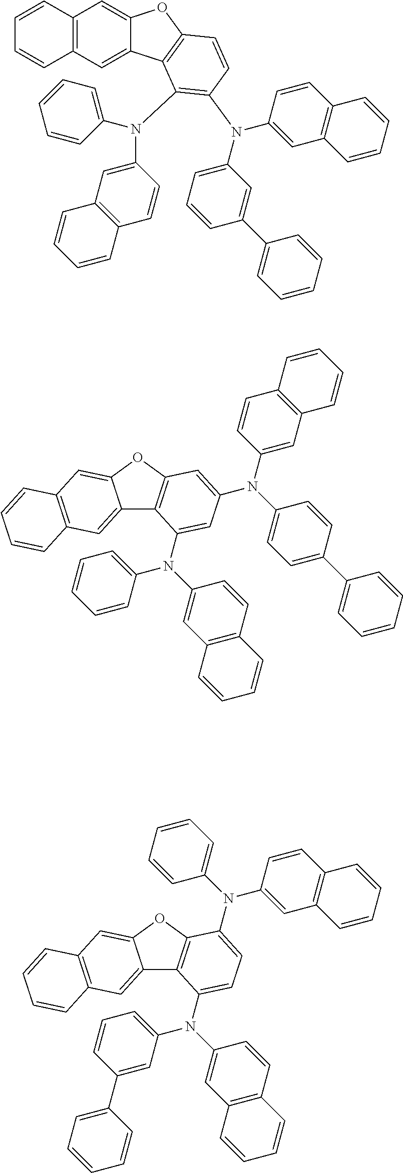 [SMILES: c1ccc(-c2cccc(N(c3ccc4ccccc4c3)c3ccc4oc5cc6ccccc6cc5c4c3N(c3ccccc3)c3ccc4ccccc4c3)c2)cc1 ; c1ccc(-c2ccc(N(c3ccc4ccccc4c3)c3cc(N(c4ccccc4)c4ccc5ccccc5c4)c4c(c3)oc3cc5ccccc5cc34)cc2)cc1 ; c1ccc(-c2cccc(N(c3ccc4ccccc4c3)c3ccc(N(c4ccccc4)c4ccc5ccccc5c4)c4oc5cc6ccccc6cc5c34)c2)cc1] 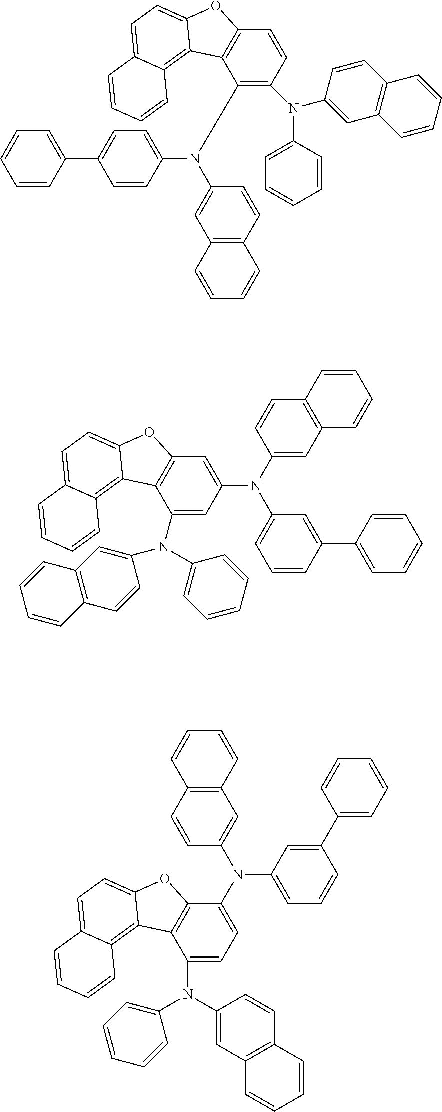 [SMILES: c1ccc(-c2ccc(N(c3ccc4ccccc4c3)c3c(N(c4ccccc4)c4ccc5ccccc5c4)ccc4oc5ccc6ccccc6c5c34)cc2)cc1 ; c1ccc(-c2cccc(N(c3ccc4ccccc4c3)c3cc(N(c4ccccc4)c4ccc5ccccc5c4)c4c(c3)oc3ccc5ccccc5c34)c2)cc1 ; c1ccc(-c2cccc(N(c3ccc4ccccc4c3)c3ccc(N(c4ccccc4)c4ccc5ccccc5c4)c4c3oc3ccc5ccccc5c34)c2)cc1] 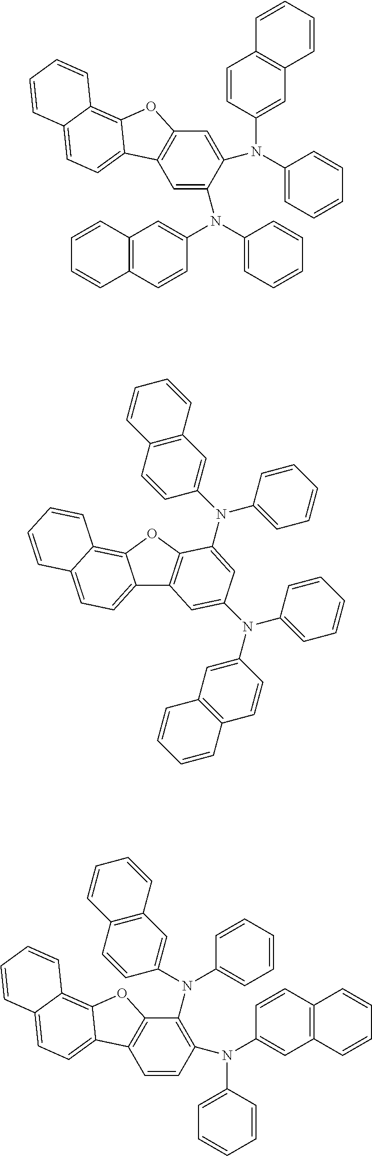 [SMILES: c1ccc(N(c2ccc3ccccc3c2)c2cc3oc4c5ccccc5ccc4c3cc2N(c2ccccc2)c2ccc3ccccc3c2)cc1 ; c1ccc(N(c2ccc3ccccc3c2)c2cc(N(c3ccccc3)c3ccc4ccccc4c3)c3oc4c5ccccc5ccc4c3c2)cc1 ; c1ccc(N(c2ccc3ccccc3c2)c2ccc3c(oc4c5ccccc5ccc34)c2N(c2ccccc2)c2ccc3ccccc3c2)cc1] 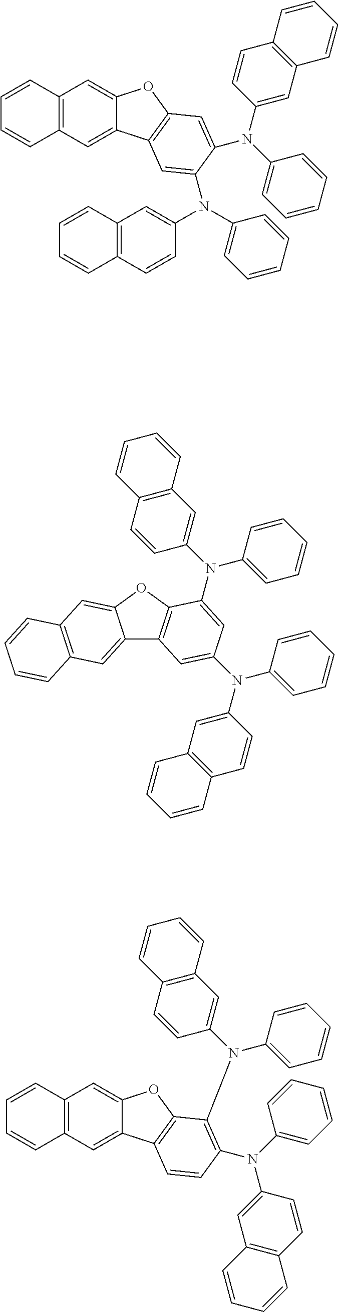 [SMILES: c1ccc(N(c2ccc3ccccc3c2)c2cc3oc4cc5ccccc5cc4c3cc2N(c2ccccc2)c2ccc3ccccc3c2)cc1 ; c1ccc(N(c2ccc3ccccc3c2)c2cc(N(c3ccccc3)c3ccc4ccccc4c3)c3oc4cc5ccccc5cc4c3c2)cc1 ; c1ccc(N(c2ccc3ccccc3c2)c2ccc3c(oc4cc5ccccc5cc43)c2N(c2ccccc2)c2ccc3ccccc3c2)cc1] 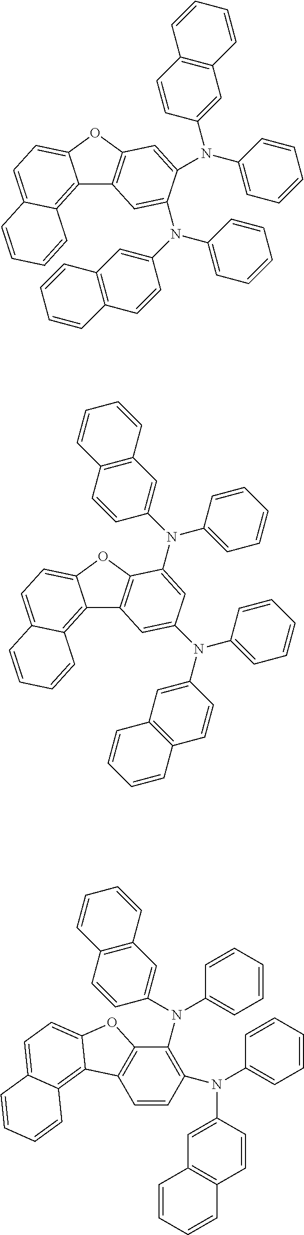 [SMILES: c1ccc(N(c2ccc3ccccc3c2)c2cc3oc4ccc5ccccc5c4c3cc2N(c2ccccc2)c2ccc3ccccc3c2)cc1 ; c1ccc(N(c2ccc3ccccc3c2)c2cc(N(c3ccccc3)c3ccc4ccccc4c3)c3oc4ccc5ccccc5c4c3c2)cc1 ; c1ccc(N(c2ccc3ccccc3c2)c2ccc3c(oc4ccc5ccccc5c43)c2N(c2ccccc2)c2ccc3ccccc3c2)cc1] 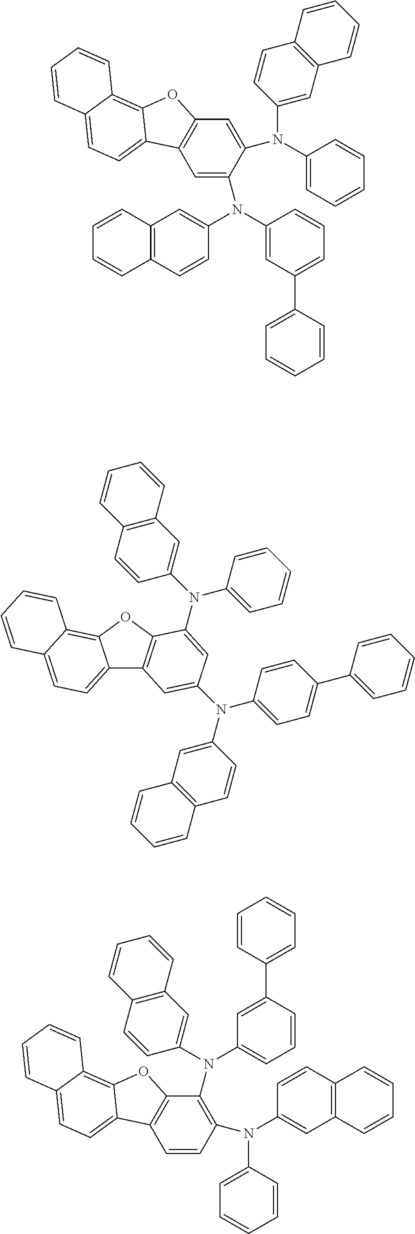 [SMILES: c1ccc(-c2cccc(N(c3ccc4ccccc4c3)c3cc4c(cc3N(c3ccccc3)c3ccc5ccccc5c3)oc3c5ccccc5ccc43)c2)cc1 ; c1ccc(-c2ccc(N(c3ccc4ccccc4c3)c3cc(N(c4ccccc4)c4ccc5ccccc5c4)c4oc5c6ccccc6ccc5c4c3)cc2)cc1 ; c1ccc(-c2cccc(N(c3ccc4ccccc4c3)c3c(N(c4ccccc4)c4ccc5ccccc5c4)ccc4c3oc3c5ccccc5ccc43)c2)cc1] 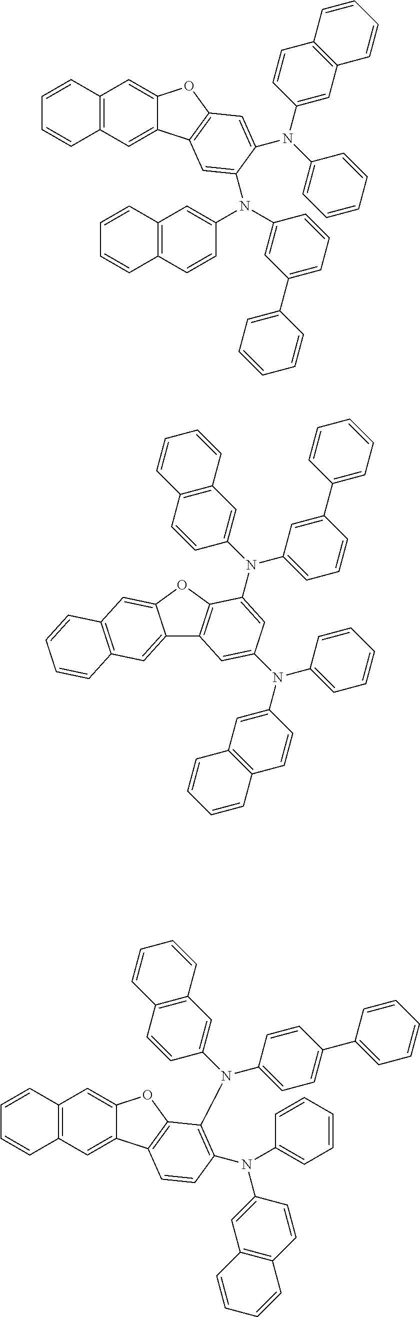 [SMILES: c1ccc(-c2cccc(N(c3ccc4ccccc4c3)c3cc4c(cc3N(c3ccccc3)c3ccc5ccccc5c3)oc3cc5ccccc5cc34)c2)cc1 ; c1ccc(-c2cccc(N(c3ccc4ccccc4c3)c3cc(N(c4ccccc4)c4ccc5ccccc5c4)cc4c3oc3cc5ccccc5cc34)c2)cc1 ; c1ccc(-c2ccc(N(c3ccc4ccccc4c3)c3c(N(c4ccccc4)c4ccc5ccccc5c4)ccc4c3oc3cc5ccccc5cc34)cc2)cc1] 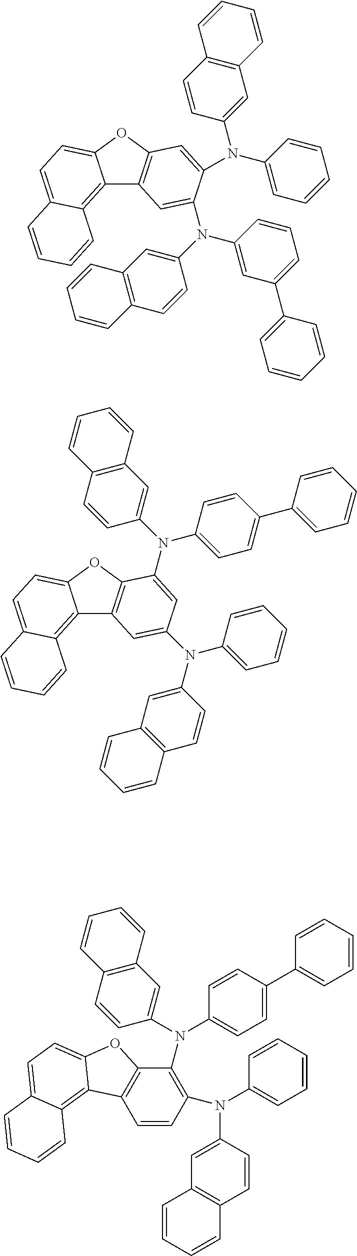 [SMILES: c1ccc(-c2cccc(N(c3ccc4ccccc4c3)c3cc4c(cc3N(c3ccccc3)c3ccc5ccccc5c3)oc3ccc5ccccc5c34)c2)cc1 ; c1ccc(-c2ccc(N(c3ccc4ccccc4c3)c3cc(N(c4ccccc4)c4ccc5ccccc5c4)cc4c3oc3ccc5ccccc5c34)cc2)cc1 ; c1ccc(-c2ccc(N(c3ccc4ccccc4c3)c3c(N(c4ccccc4)c4ccc5ccccc5c4)ccc4c3oc3ccc5ccccc5c34)cc2)cc1] 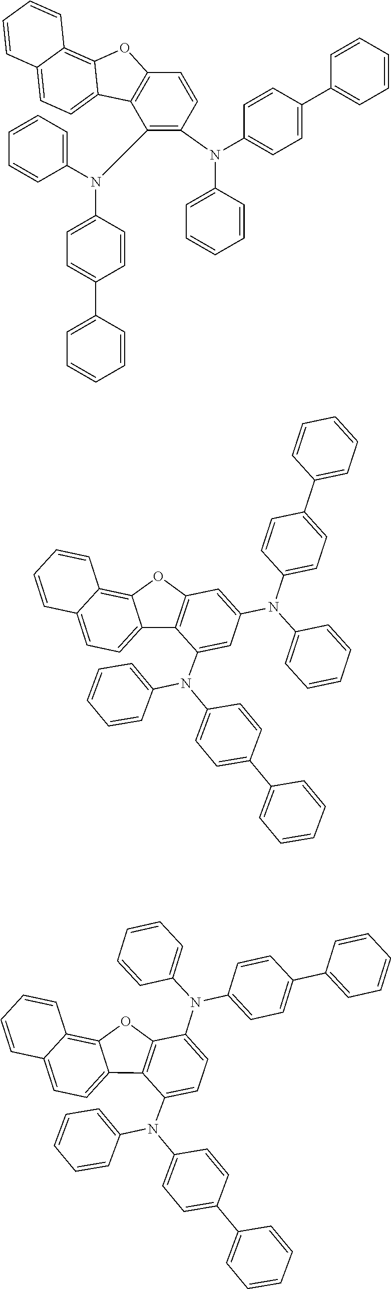 [SMILES: c1ccc(-c2ccc(N(c3ccccc3)c3ccc4oc5c6ccccc6ccc5c4c3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4c(c3)oc3c5ccccc5ccc34)cc2)cc1 ; c1ccc(-c2ccc(N(c3ccccc3)c3ccc(N(c4ccccc4)c4ccc(-c5ccccc5)cc4)c4c3oc3c5ccccc5ccc34)cc2)cc1] 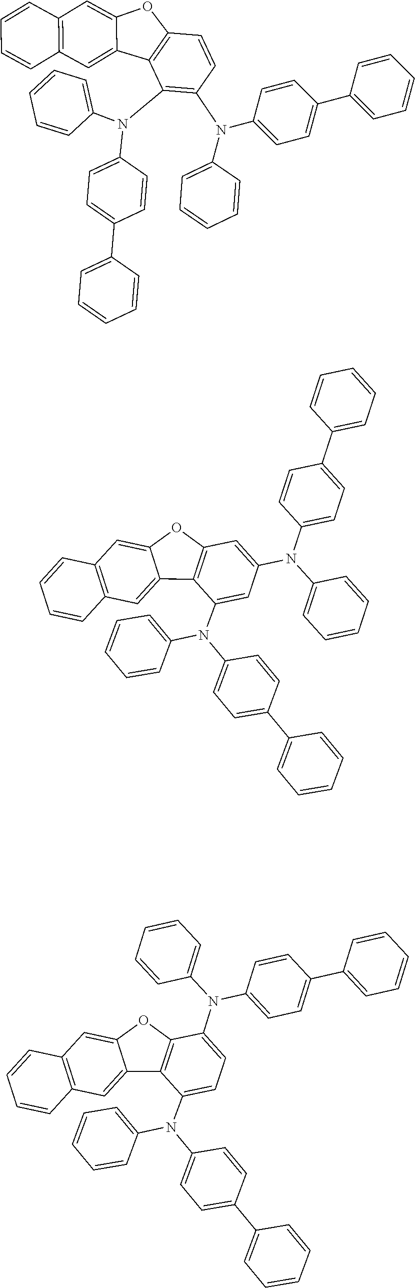 [SMILES: c1ccc(-c2ccc(N(c3ccccc3)c3ccc4oc5cc6ccccc6cc5c4c3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4c(c3)oc3cc5ccccc5cc34)cc2)cc1 ; c1ccc(-c2ccc(N(c3ccccc3)c3ccc(N(c4ccccc4)c4ccc(-c5ccccc5)cc4)c4c3oc3cc5ccccc5cc34)cc2)cc1] 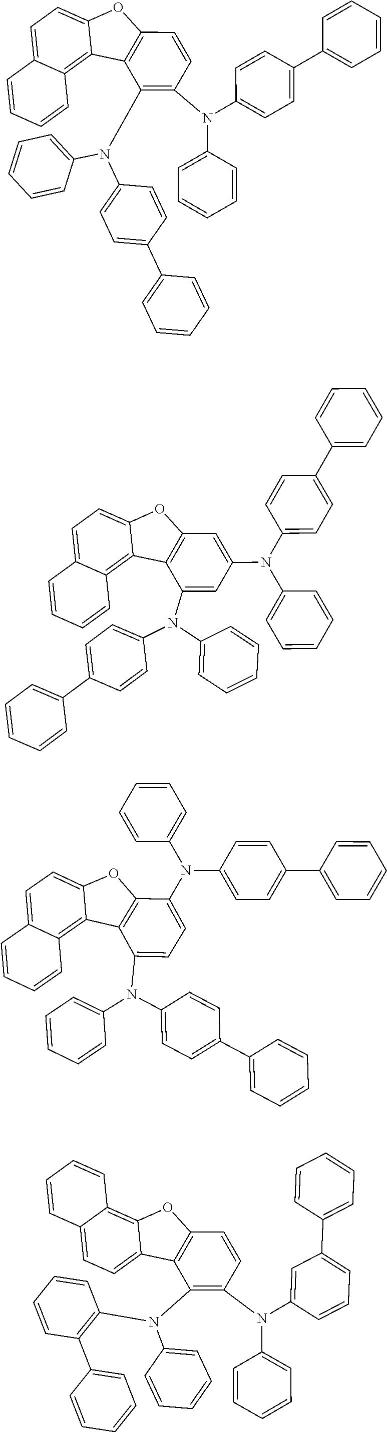 [SMILES: c1ccc(-c2ccc(N(c3ccccc3)c3ccc4oc5ccc6ccccc6c5c4c3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4c(c3)oc3ccc5ccccc5c34)cc2)cc1 ; c1ccc(-c2ccc(N(c3ccccc3)c3ccc(N(c4ccccc4)c4ccc(-c5ccccc5)cc4)c4c3oc3ccc5ccccc5c34)cc2)cc1 ; c1ccc(-c2cccc(N(c3ccccc3)c3ccc4oc5c6ccccc6ccc5c4c3N(c3ccccc3)c3ccccc3-c3ccccc3)c2)cc1] 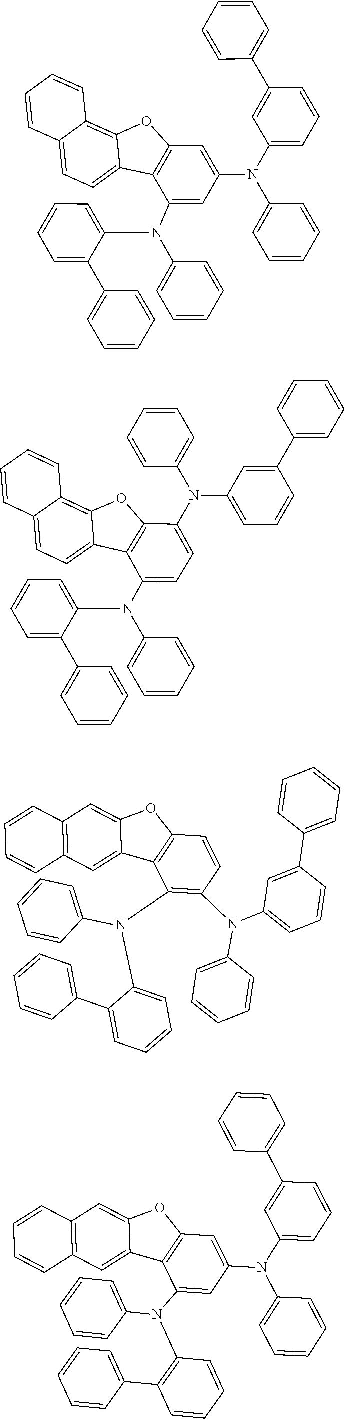 [SMILES: c1ccc(-c2cccc(N(c3ccccc3)c3cc(N(c4ccccc4)c4ccccc4-c4ccccc4)c4c(c3)oc3c5ccccc5ccc34)c2)cc1 ; c1ccc(-c2cccc(N(c3ccccc3)c3ccc(N(c4ccccc4)c4ccccc4-c4ccccc4)c4c3oc3c5ccccc5ccc34)c2)cc1 ; c1ccc(-c2cccc(N(c3ccccc3)c3ccc4oc5cc6ccccc6cc5c4c3N(c3ccccc3)c3ccccc3-c3ccccc3)c2)cc1 ; c1ccc(-c2cccc(N(c3ccccc3)c3cc(N(c4ccccc4)c4ccccc4-c4ccccc4)c4c(c3)oc3cc5ccccc5cc34)c2)cc1] 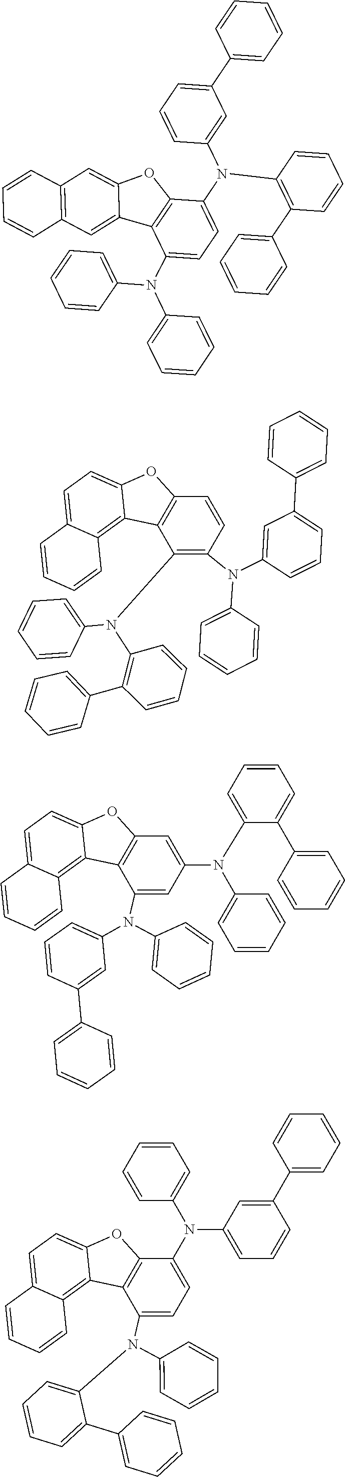 [SMILES: c1ccc(-c2cccc(N(c3ccccc3-c3ccccc3)c3ccc(N(c4ccccc4)c4ccccc4)c4c3oc3cc5ccccc5cc34)c2)cc1 ; c1ccc(-c2cccc(N(c3ccccc3)c3ccc4oc5ccc6ccccc6c5c4c3N(c3ccccc3)c3ccccc3-c3ccccc3)c2)cc1 ; c1ccc(-c2cccc(N(c3ccccc3)c3cc(N(c4ccccc4)c4ccccc4-c4ccccc4)cc4oc5ccc6ccccc6c5c34)c2)cc1 ; c1ccc(-c2cccc(N(c3ccccc3)c3ccc(N(c4ccccc4)c4ccccc4-c4ccccc4)c4c3oc3ccc5ccccc5c34)c2)cc1] 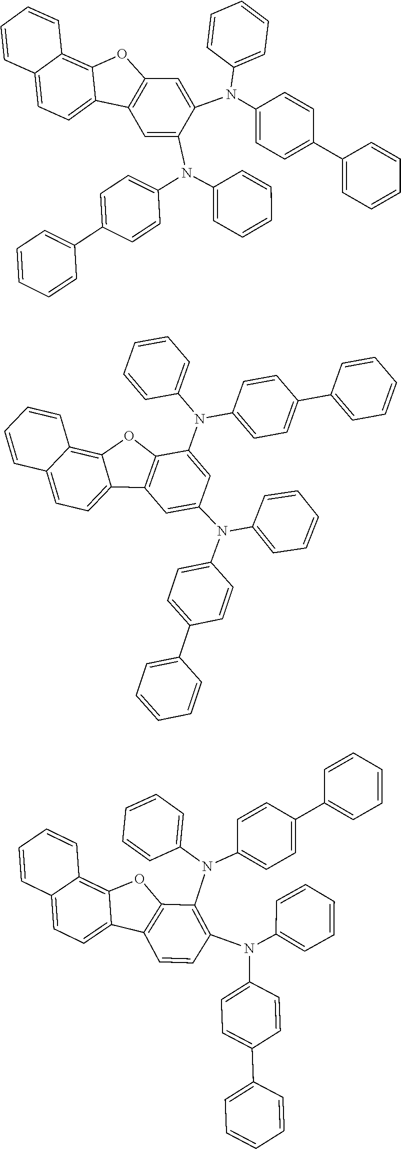 [SMILES: c1ccc(-c2ccc(N(c3ccccc3)c3cc4oc5c6ccccc6ccc5c4cc3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4oc5c6ccccc6ccc5c4c3)cc2)cc1 ; c1ccc(-c2ccc(N(c3ccccc3)c3ccc4c(oc5c6ccccc6ccc45)c3N(c3ccccc3)c3ccc(-c4ccccc4)cc3)cc2)cc1] 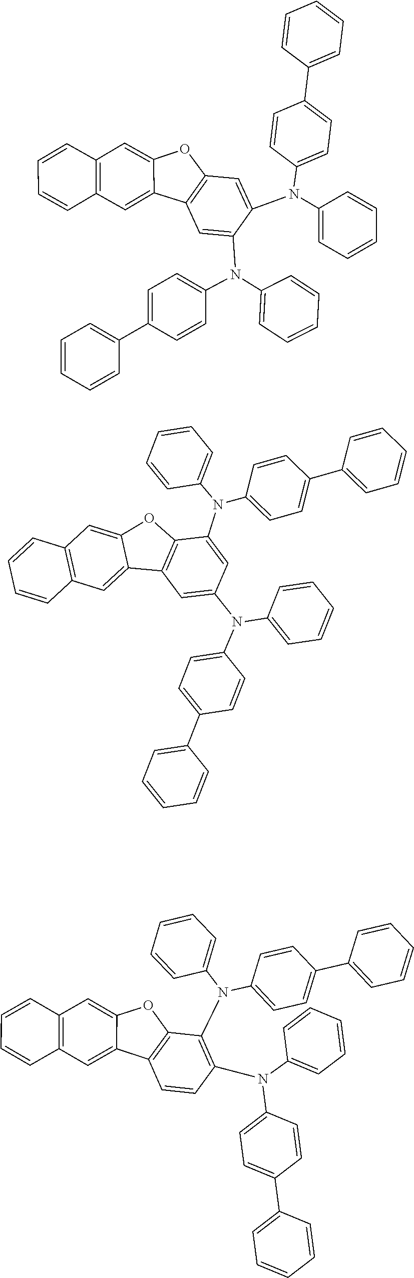 [SMILES: c1ccc(-c2ccc(N(c3ccccc3)c3cc4oc5cc6ccccc6cc5c4cc3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4oc5cc6ccccc6cc5c4c3)cc2)cc1 ; c1ccc(-c2ccc(N(c3ccccc3)c3ccc4c(oc5cc6ccccc6cc54)c3N(c3ccccc3)c3ccc(-c4ccccc4)cc3)cc2)cc1] 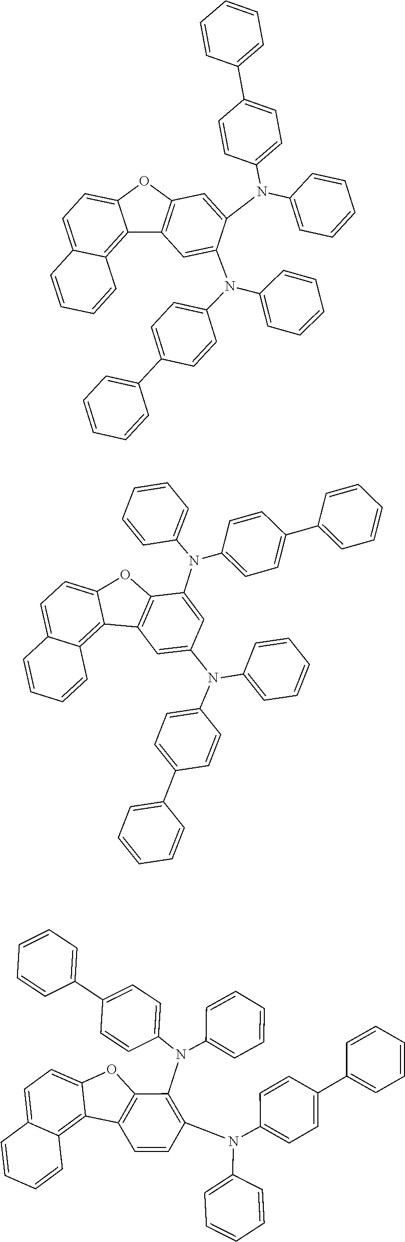 [SMILES: c1ccc(-c2ccc(N(c3ccccc3)c3cc4oc5ccc6ccccc6c5c4cc3N(c3ccccc3)c3ccc(-c4ccccc4)cc3)cc2)cc1 ; c1ccc(-c2ccc(N(c3ccccc3)c3cc(N(c4ccccc4)c4ccc(-c5ccccc5)cc4)c4oc5ccc6ccccc6c5c4c3)cc2)cc1 ; c1ccc(-c2ccc(N(c3ccccc3)c3ccc4c(oc5ccc6ccccc6c54)c3N(c3ccccc3)c3ccc(-c4ccccc4)cc3)cc2)cc1] 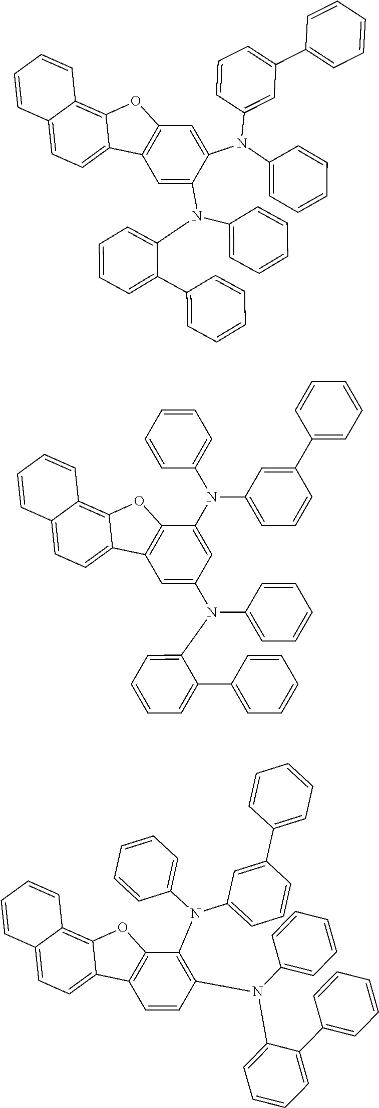 [SMILES: c1ccc(-c2cccc(N(c3ccccc3)c3cc4oc5c6ccccc6ccc5c4cc3N(c3ccccc3)c3ccccc3-c3ccccc3)c2)cc1 ; c1ccc(-c2cccc(N(c3ccccc3)c3cc(N(c4ccccc4)c4ccccc4-c4ccccc4)cc4c3oc3c5ccccc5ccc43)c2)cc1 ; c1ccc(-c2cccc(N(c3ccccc3)c3c(N(c4ccccc4)c4ccccc4-c4ccccc4)ccc4c3oc3c5ccccc5ccc43)c2)cc1] 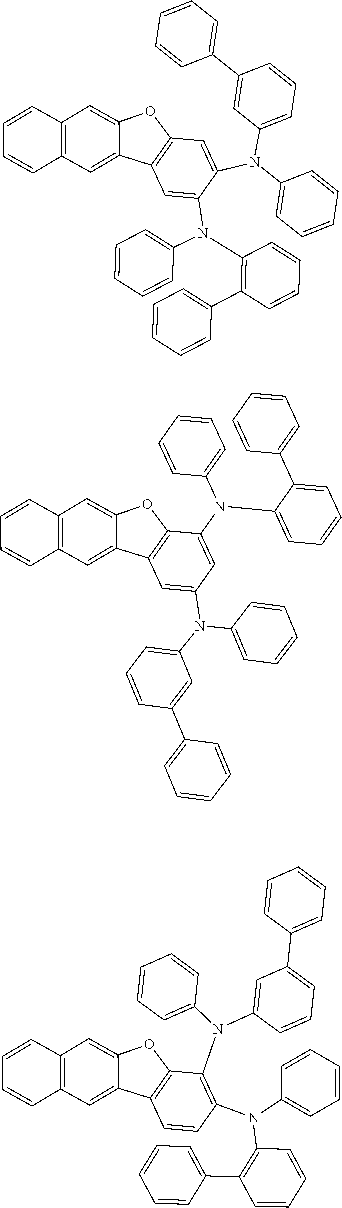 [SMILES: c1ccc(-c2cccc(N(c3ccccc3)c3cc4oc5cc6ccccc6cc5c4cc3N(c3ccccc3)c3ccccc3-c3ccccc3)c2)cc1 ; c1ccc(-c2cccc(N(c3ccccc3)c3cc(N(c4ccccc4)c4ccccc4-c4ccccc4)c4oc5cc6ccccc6cc5c4c3)c2)cc1 ; c1ccc(-c2cccc(N(c3ccccc3)c3c(N(c4ccccc4)c4ccccc4-c4ccccc4)ccc4c3oc3cc5ccccc5cc34)c2)cc1] 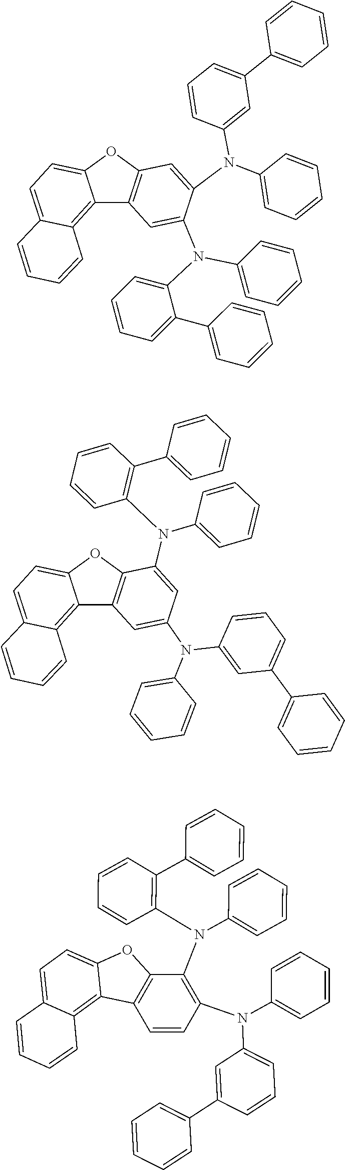 [SMILES: c1ccc(-c2cccc(N(c3ccccc3)c3cc4oc5ccc6ccccc6c5c4cc3N(c3ccccc3)c3ccccc3-c3ccccc3)c2)cc1 ; c1ccc(-c2cccc(N(c3ccccc3)c3cc(N(c4ccccc4)c4ccccc4-c4ccccc4)c4oc5ccc6ccccc6c5c4c3)c2)cc1 ; c1ccc(-c2cccc(N(c3ccccc3)c3ccc4c(oc5ccc6ccccc6c54)c3N(c3ccccc3)c3ccccc3-c3ccccc3)c2)cc1] 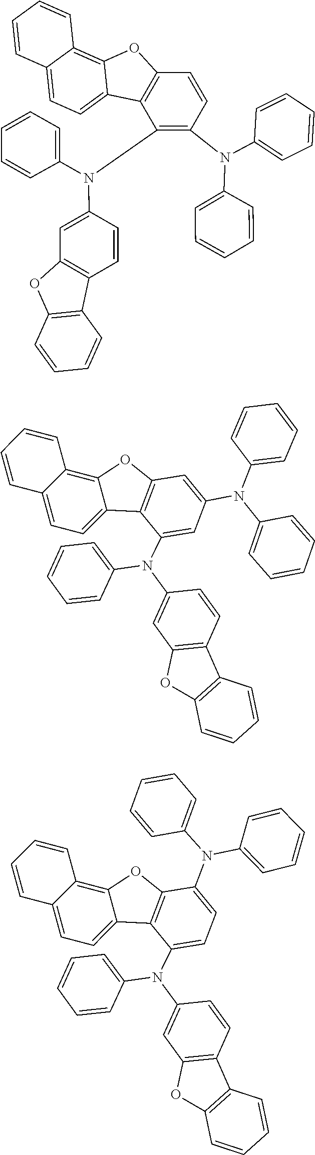 [SMILES: c1ccc(N(c2ccccc2)c2ccc3oc4c5ccccc5ccc4c3c2N(c2ccccc2)c2ccc3c(c2)oc2ccccc23)cc1 ; c1ccc(N(c2ccccc2)c2cc(N(c3ccccc3)c3ccc4c(c3)oc3ccccc34)c3c(c2)oc2c4ccccc4ccc23)cc1 ; c1ccc(N(c2ccccc2)c2ccc(N(c3ccccc3)c3ccc4c(c3)oc3ccccc34)c3c2oc2c4ccccc4ccc23)cc1] 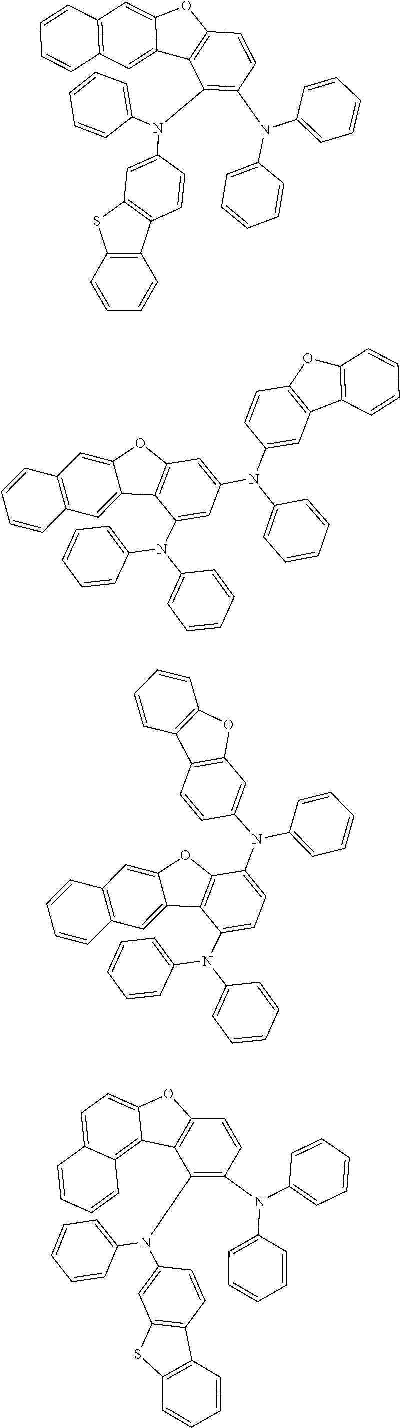 [SMILES: c1ccc(N(c2ccccc2)c2ccc3oc4cc5ccccc5cc4c3c2N(c2ccccc2)c2ccc3c(c2)sc2ccccc23)cc1 ; c1ccc(N(c2cc(N(c3ccccc3)c3ccccc3)c3c(c2)oc2cc4ccccc4cc23)c2ccc3oc4ccccc4c3c2)cc1 ; c1ccc(N(c2ccc3c(c2)oc2ccccc23)c2ccc(N(c3ccccc3)c3ccccc3)c3c2oc2cc4ccccc4cc23)cc1 ; c1ccc(N(c2ccccc2)c2ccc3oc4ccc5ccccc5c4c3c2N(c2ccccc2)c2ccc3c(c2)sc2ccccc23)cc1] 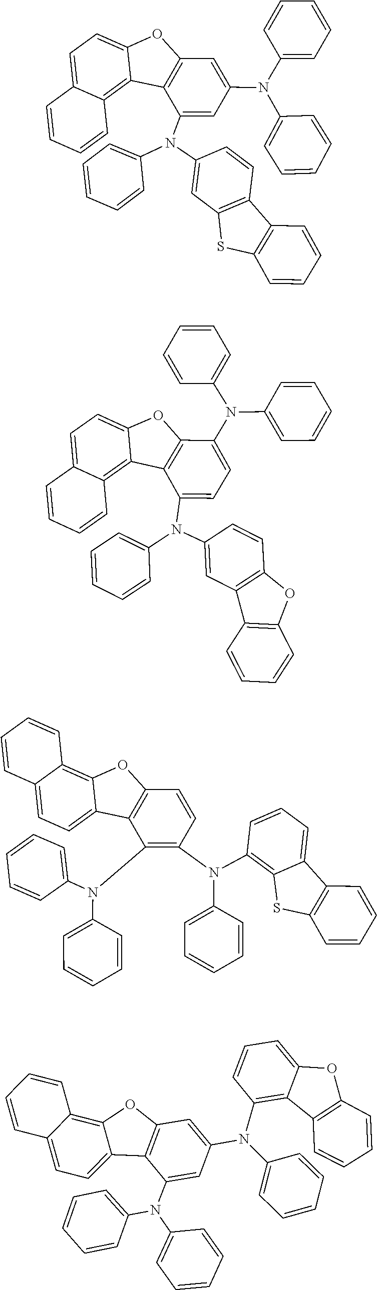 [SMILES: c1ccc(N(c2ccccc2)c2cc(N(c3ccccc3)c3ccc4c(c3)sc3ccccc34)c3c(c2)oc2ccc4ccccc4c23)cc1 ; c1ccc(N(c2ccccc2)c2ccc(N(c3ccccc3)c3ccc4oc5ccccc5c4c3)c3c2oc2ccc4ccccc4c23)cc1 ; c1ccc(N(c2ccc3oc4c5ccccc5ccc4c3c2N(c2ccccc2)c2ccccc2)c2cccc3c2sc2ccccc23)cc1 ; c1ccc(N(c2cc(N(c3ccccc3)c3ccccc3)c3c(c2)oc2c4ccccc4ccc23)c2cccc3oc4ccccc4c23)cc1] 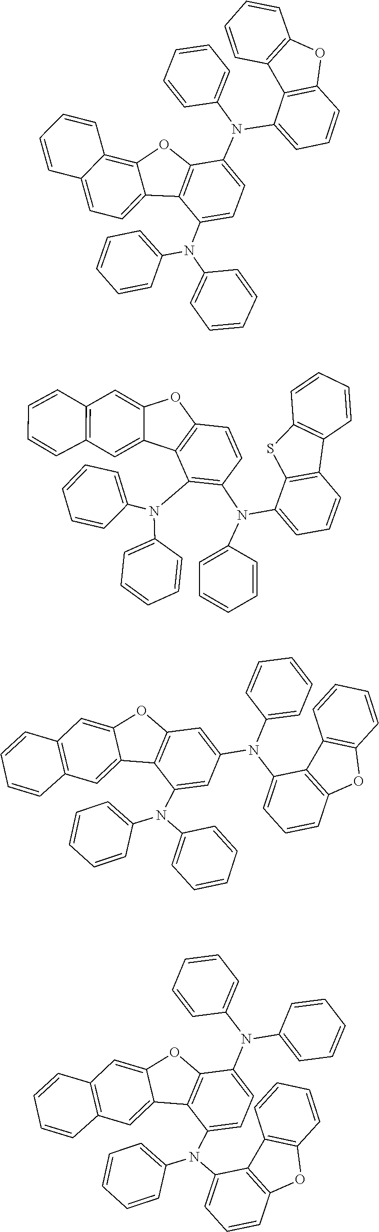 [SMILES: c1ccc(N(c2ccc(N(c3ccccc3)c3ccccc3)c3c2oc2c4ccccc4ccc23)c2cccc3oc4ccccc4c23)cc1 ; c1ccc(N(c2ccc3oc4cc5ccccc5cc4c3c2N(c2ccccc2)c2ccccc2)c2cccc3c2sc2ccccc23)cc1 ; c1ccc(N(c2cc(N(c3ccccc3)c3ccccc3)c3c(c2)oc2cc4ccccc4cc23)c2cccc3oc4ccccc4c23)cc1 ; c1ccc(N(c2ccccc2)c2ccc(N(c3ccccc3)c3cccc4oc5ccccc5c34)c3c2oc2cc4ccccc4cc23)cc1] 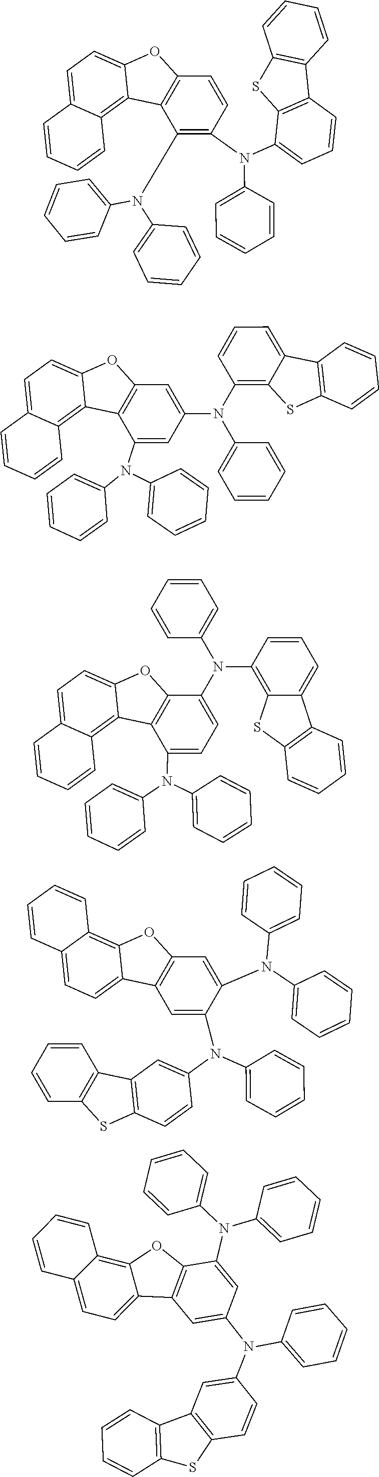 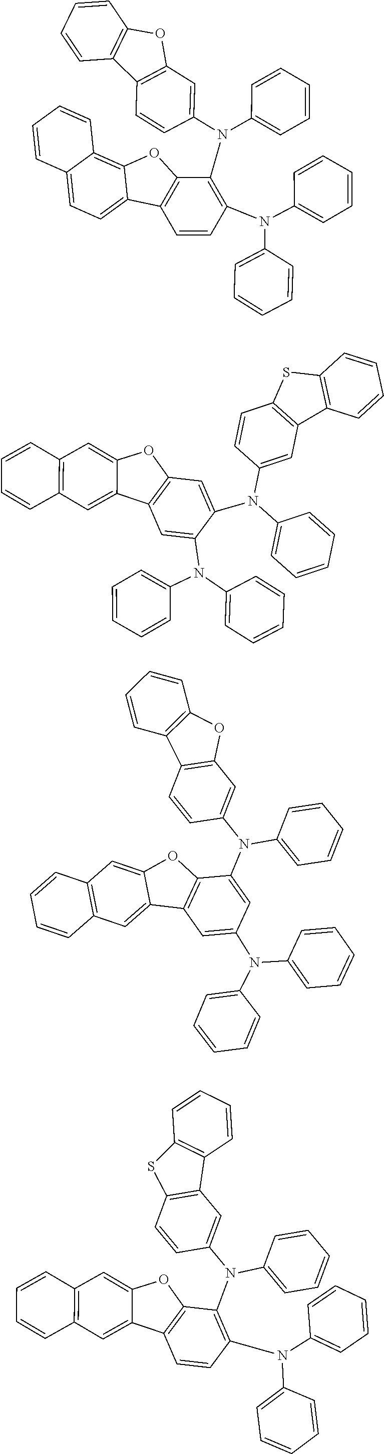 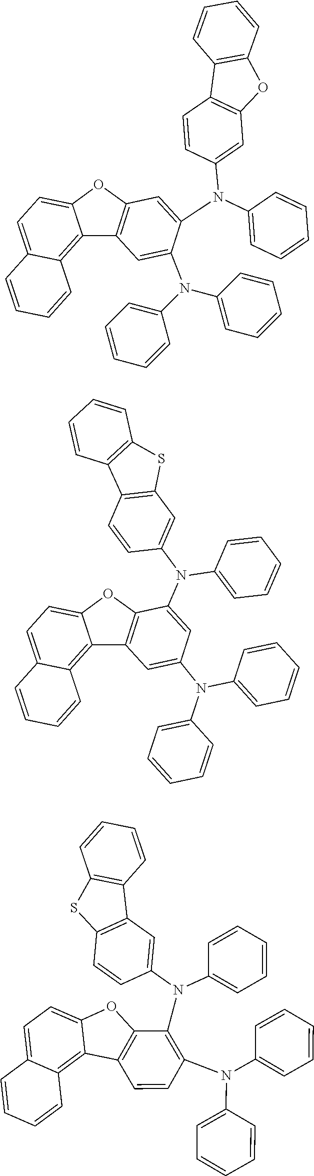 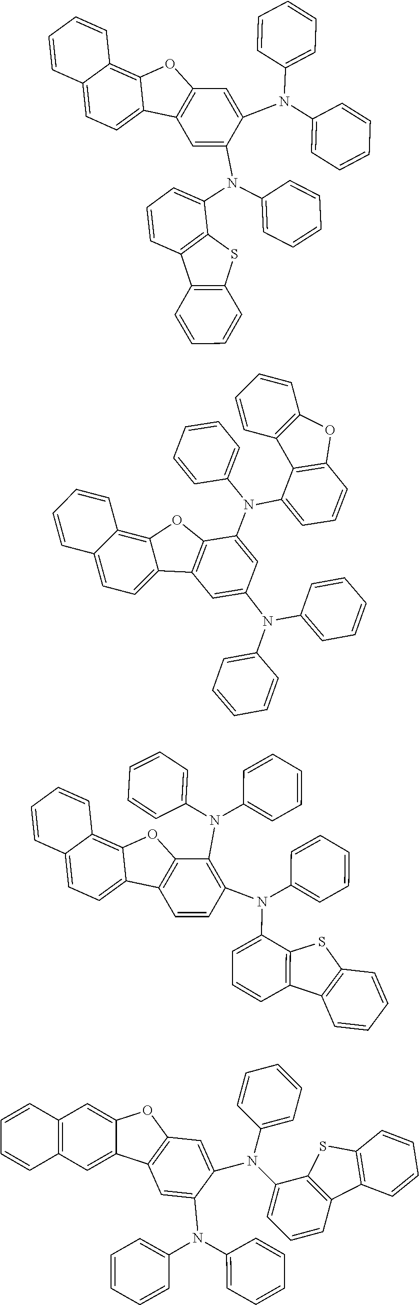 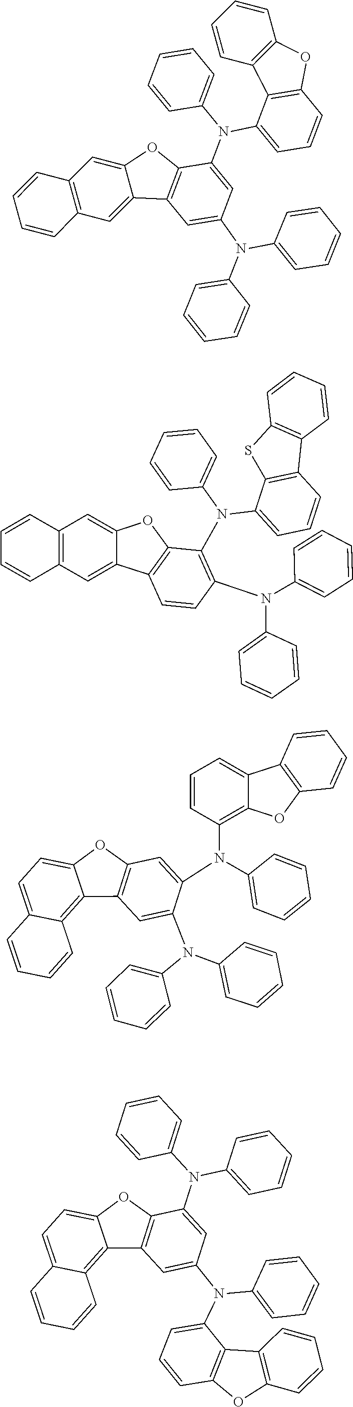 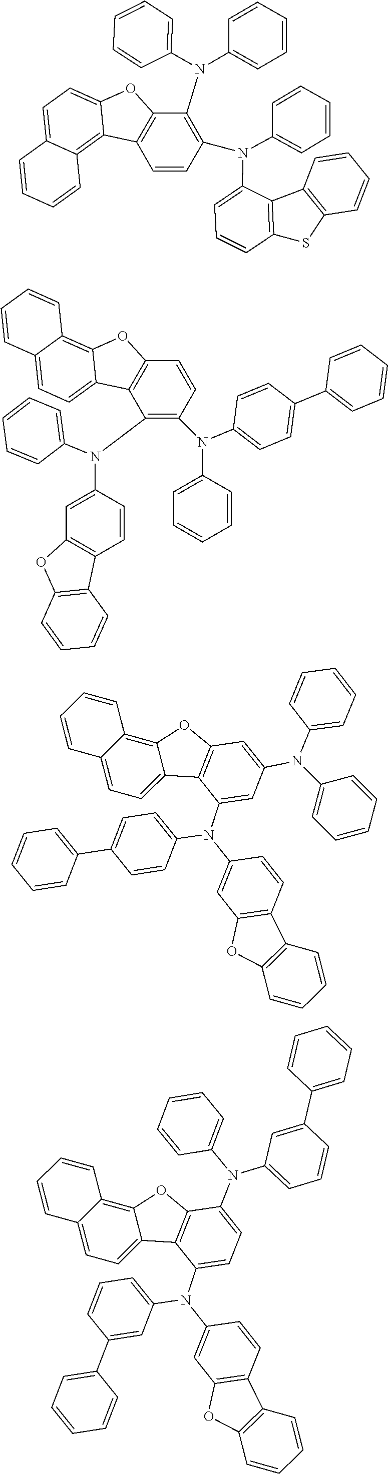 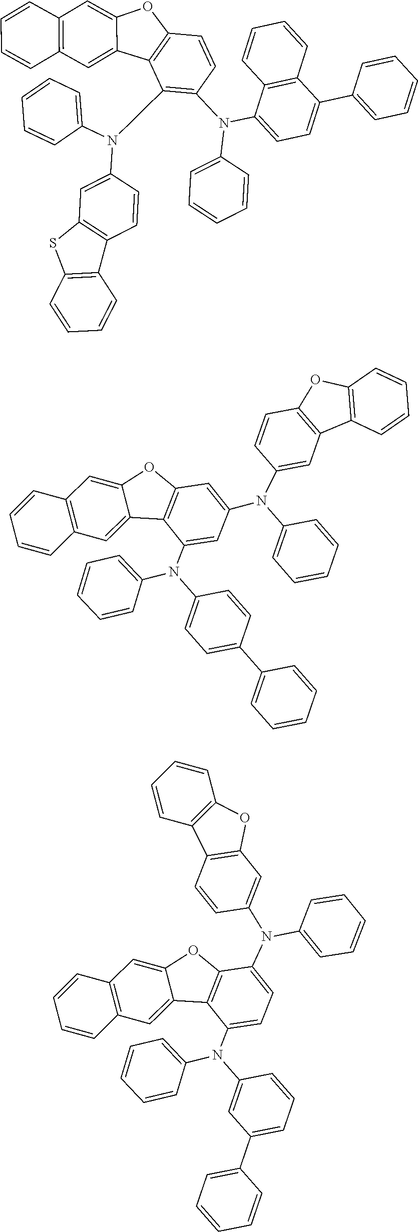 [SMILES: c1ccc(-c2ccc(N(c3ccccc3)c3ccc4oc5cc6ccccc6cc5c4c3N(c3ccccc3)c3ccc4c(c3)sc3ccccc34)c3ccccc23)cc1 ; c1ccc(-c2ccc(N(c3ccccc3)c3cc(N(c4ccccc4)c4ccc5oc6ccccc6c5c4)cc4oc5cc6ccccc6cc5c34)cc2)cc1 ; c1ccc(-c2cccc(N(c3ccccc3)c3ccc(N(c4ccccc4)c4ccc5c(c4)oc4ccccc45)c4oc5cc6ccccc6cc5c34)c2)cc1] 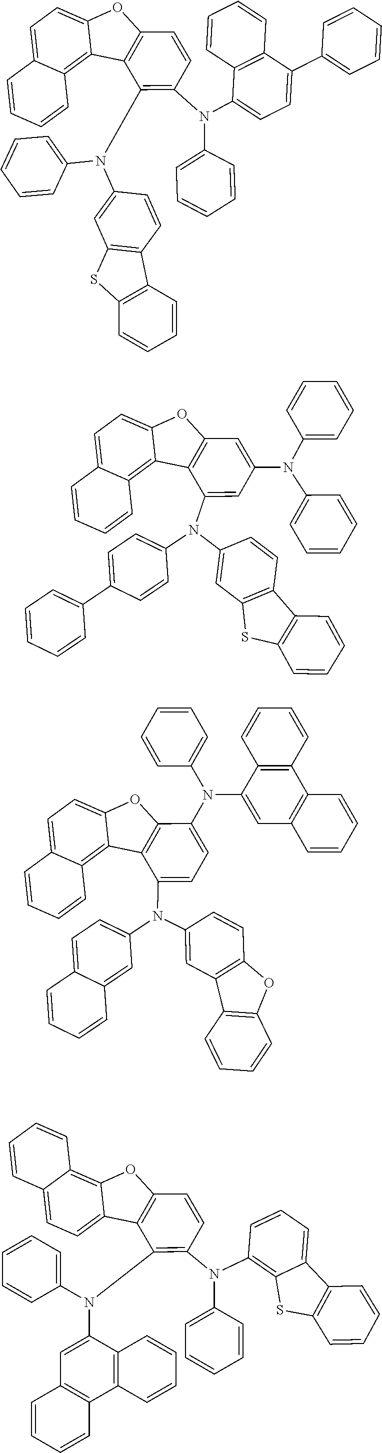 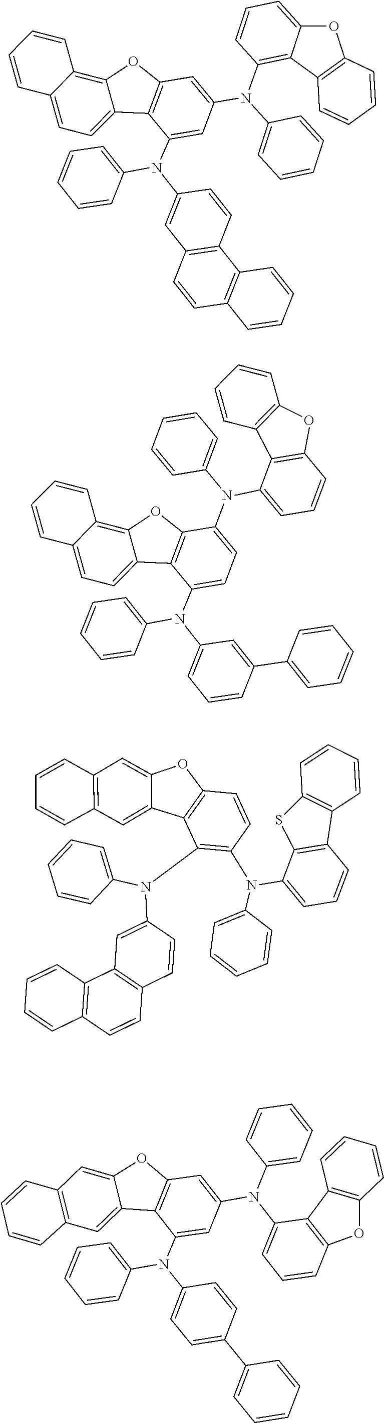 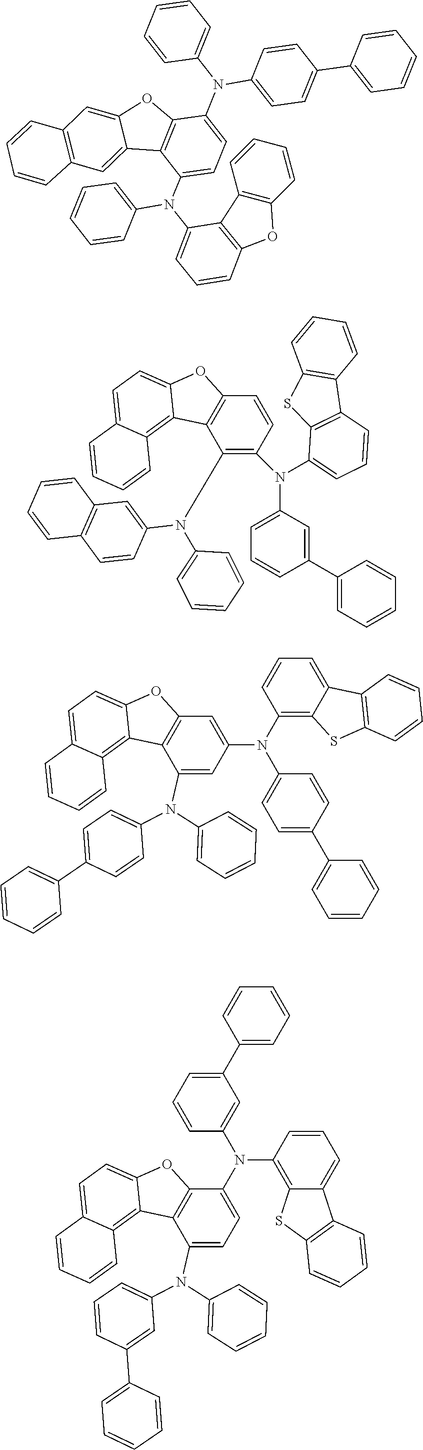 [SMILES: c1ccc(-c2ccc(N(c3ccccc3)c3ccc(N(c4ccccc4)c4cccc5oc6ccccc6c45)c4c3oc3cc5ccccc5cc34)cc2)cc1 ; c1ccc(-c2cccc(N(c3ccc4oc5ccc6ccccc6c5c4c3N(c3ccccc3)c3ccc4ccccc4c3)c3cccc4c3sc3ccccc34)c2)cc1 ; c1ccc(-c2ccc(N(c3cc(N(c4ccccc4)c4ccc(-c5ccccc5)cc4)c4c(c3)oc3ccc5ccccc5c34)c3cccc4c3sc3ccccc34)cc2)cc1 ; c1ccc(-c2cccc(N(c3ccc(N(c4ccccc4)c4cccc(-c5ccccc5)c4)c4c3oc3ccc5ccccc5c34)c3cccc4c3sc3ccccc34)c2)cc1] 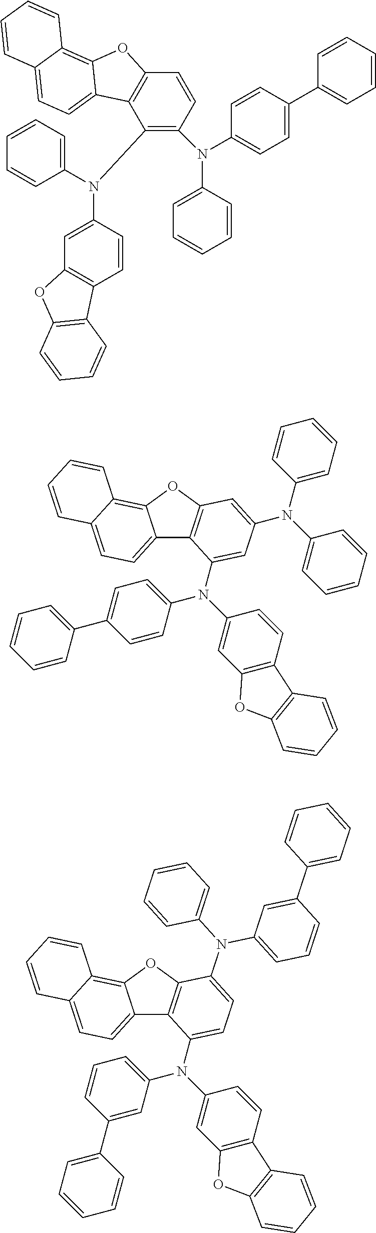 [SMILES: c1ccc(-c2ccc(N(c3ccccc3)c3ccc4oc5c6ccccc6ccc5c4c3N(c3ccccc3)c3ccc4c(c3)oc3ccccc34)cc2)cc1 ; c1ccc(-c2ccc(N(c3ccc4c(c3)oc3ccccc34)c3cc(N(c4ccccc4)c4ccccc4)cc4oc5c6ccccc6ccc5c34)cc2)cc1 ; c1ccc(-c2cccc(N(c3ccccc3)c3ccc(N(c4cccc(-c5ccccc5)c4)c4ccc5c(c4)oc4ccccc45)c4c3oc3c5ccccc5ccc34)c2)cc1] 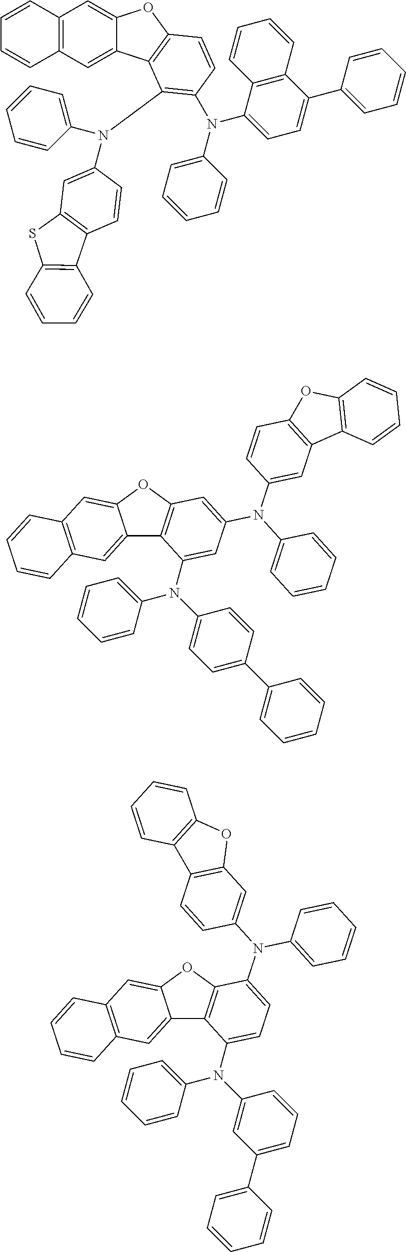 [SMILES: c1ccc(-c2ccc(N(c3ccccc3)c3ccc4oc5cc6ccccc6cc5c4c3N(c3ccccc3)c3ccc4c(c3)sc3ccccc34)c3ccccc23)cc1 ; c1ccc(-c2ccc(N(c3ccccc3)c3cc(N(c4ccccc4)c4ccc5oc6ccccc6c5c4)cc4oc5cc6ccccc6cc5c34)cc2)cc1 ; c1ccc(-c2cccc(N(c3ccccc3)c3ccc(N(c4ccccc4)c4ccc5c(c4)oc4ccccc45)c4oc5cc6ccccc6cc5c34)c2)cc1] 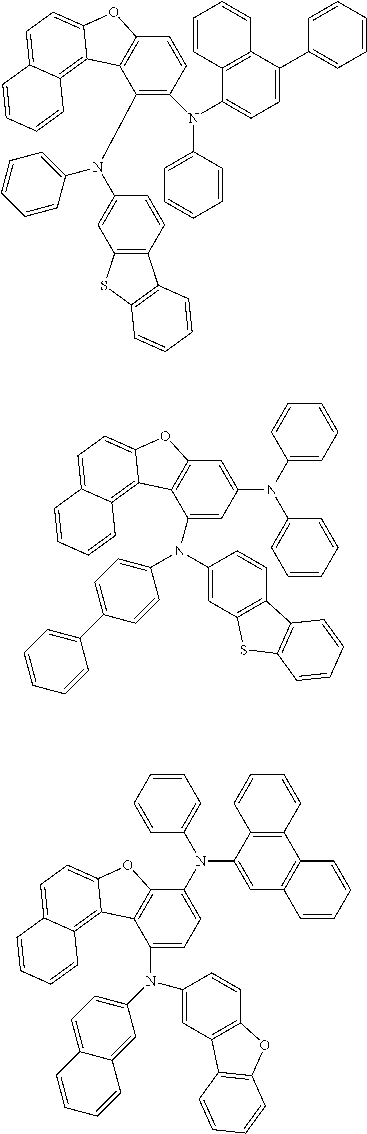 [SMILES: c1ccc(-c2ccc(N(c3ccccc3)c3ccc4oc5ccc6ccccc6c5c4c3N(c3ccccc3)c3ccc4c(c3)sc3ccccc34)c3ccccc23)cc1 ; c1ccc(-c2ccc(N(c3ccc4c(c3)sc3ccccc34)c3cc(N(c4ccccc4)c4ccccc4)cc4oc5ccc6ccccc6c5c34)cc2)cc1 ; c1ccc(N(c2cc3ccccc3c3ccccc23)c2ccc(N(c3ccc4ccccc4c3)c3ccc4oc5ccccc5c4c3)c3c2oc2ccc4ccccc4c23)cc1] 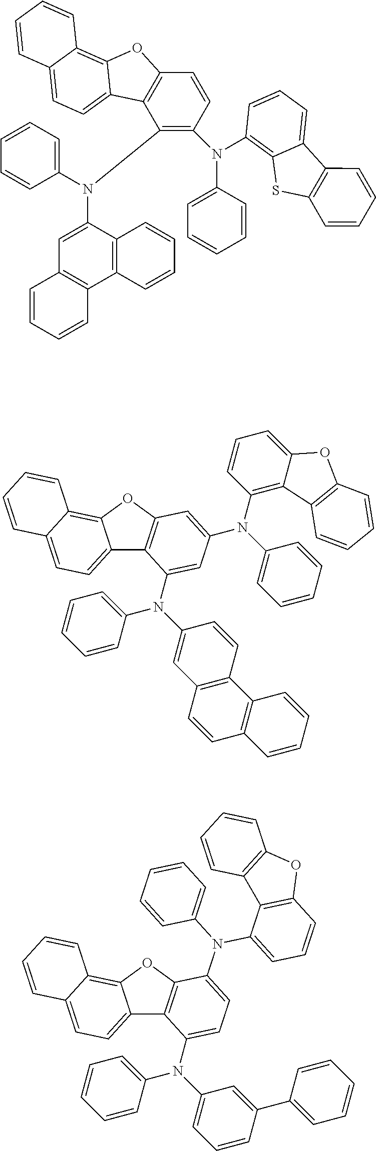 [SMILES: c1ccc(N(c2ccc3oc4c5ccccc5ccc4c3c2N(c2ccccc2)c2cc3ccccc3c3ccccc23)c2cccc3c2sc2ccccc23)cc1 ; c1ccc(N(c2cc(N(c3ccccc3)c3ccc4c(ccc5ccccc54)c3)c3c(c2)oc2c4ccccc4ccc23)c2cccc3oc4ccccc4c23)cc1 ; c1ccc(-c2cccc(N(c3ccccc3)c3ccc(N(c4ccccc4)c4cccc5oc6ccccc6c45)c4oc5c6ccccc6ccc5c34)c2)cc1] 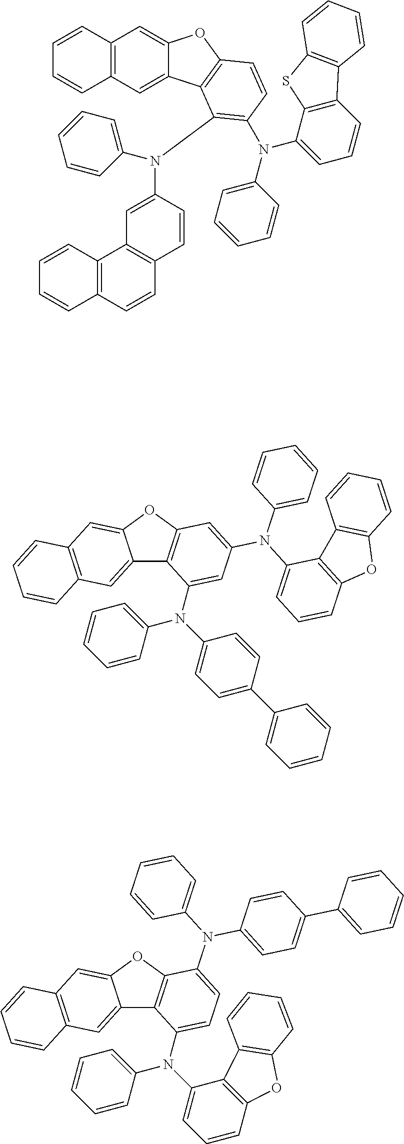 [SMILES: c1ccc(N(c2ccc3oc4cc5ccccc5cc4c3c2N(c2ccccc2)c2ccc3ccc4ccccc4c3c2)c2cccc3c2sc2ccccc23)cc1 ; c1ccc(-c2ccc(N(c3ccccc3)c3cc(N(c4ccccc4)c4cccc5oc6ccccc6c45)cc4oc5cc6ccccc6cc5c34)cc2)cc1 ; c1ccc(-c2ccc(N(c3ccccc3)c3ccc(N(c4ccccc4)c4cccc5oc6ccccc6c45)c4c3oc3cc5ccccc5cc34)cc2)cc1] 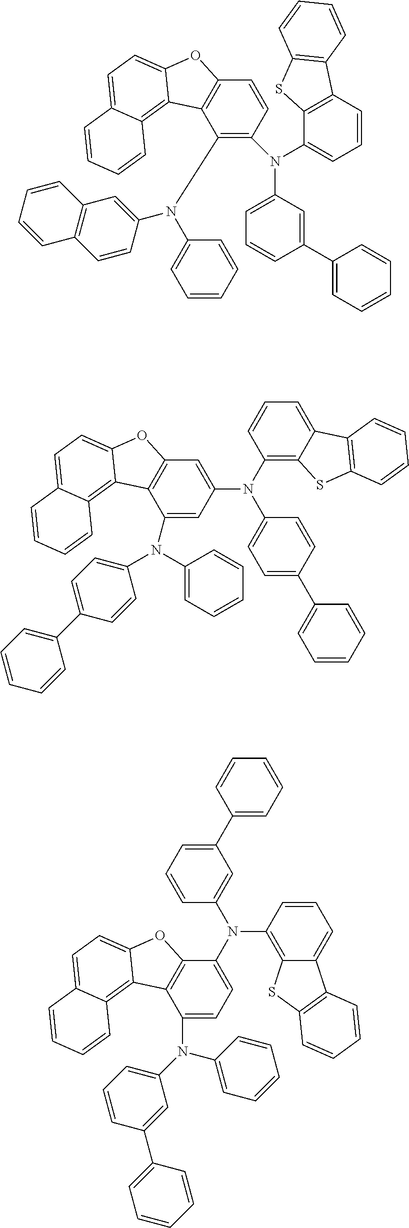 [SMILES: c1ccc(-c2cccc(N(c3ccc4oc5ccc6ccccc6c5c4c3N(c3ccccc3)c3ccc4ccccc4c3)c3cccc4c3sc3ccccc34)c2)cc1 ; c1ccc(-c2ccc(N(c3cc(N(c4ccccc4)c4ccc(-c5ccccc5)cc4)c4c(c3)oc3ccc5ccccc5c34)c3cccc4c3sc3ccccc34)cc2)cc1 ; c1ccc(-c2cccc(N(c3ccc(N(c4ccccc4)c4cccc(-c5ccccc5)c4)c4c3oc3ccc5ccccc5c34)c3cccc4c3sc3ccccc34)c2)cc1] 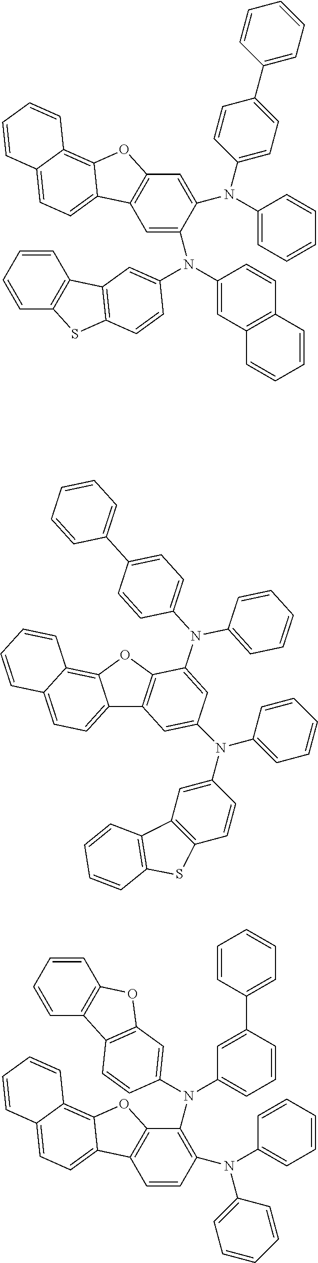 [SMILES: c1ccc(-c2ccc(N(c3ccccc3)c3cc4oc5c6ccccc6ccc5c4cc3N(c3ccc4ccccc4c3)c3ccc4sc5ccccc5c4c3)cc2)cc1 ; c1ccc(-c2ccc(N(c3ccccc3)c3cc(N(c4ccccc4)c4ccc5sc6ccccc6c5c4)cc4c3oc3c5ccccc5ccc43)cc2)cc1 ; c1ccc(-c2cccc(N(c3ccc4c(c3)oc3ccccc34)c3c(N(c4ccccc4)c4ccccc4)ccc4c3oc3c5ccccc5ccc43)c2)cc1] 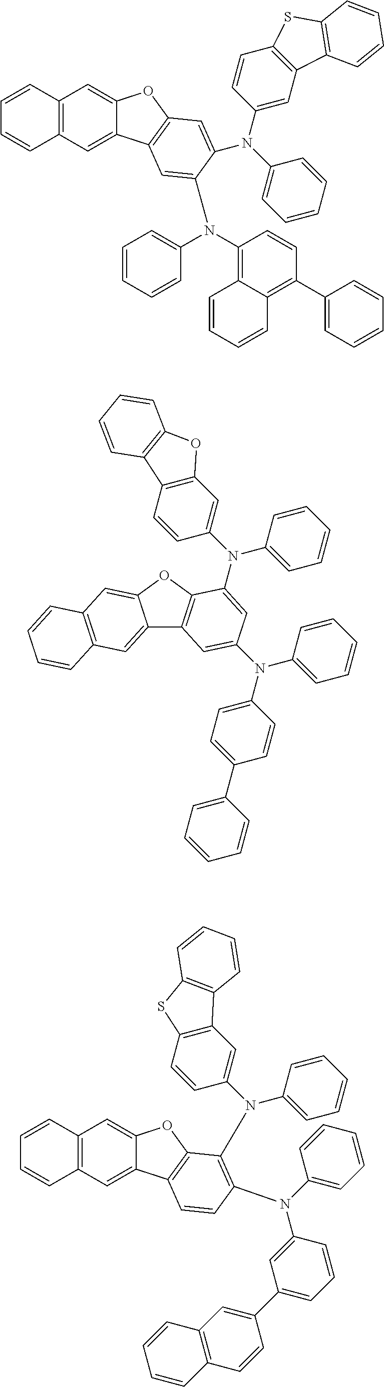 [SMILES: c1ccc(-c2ccc(N(c3ccccc3)c3cc4c(cc3N(c3ccccc3)c3ccc5sc6ccccc6c5c3)oc3cc5ccccc5cc34)c3ccccc23)cc1 ; c1ccc(-c2ccc(N(c3ccccc3)c3cc(N(c4ccccc4)c4ccc5c(c4)oc4ccccc45)c4oc5cc6ccccc6cc5c4c3)cc2)cc1 ; c1ccc(N(c2cccc(-c3ccc4ccccc4c3)c2)c2ccc3c(oc4cc5ccccc5cc43)c2N(c2ccccc2)c2ccc3sc4ccccc4c3c2)cc1] 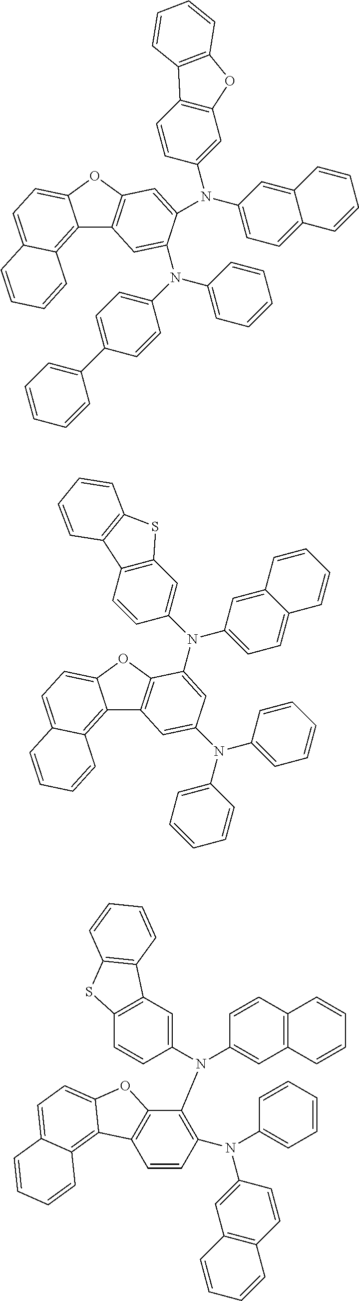 [SMILES: c1ccc(-c2ccc(N(c3ccccc3)c3cc4c(cc3N(c3ccc5ccccc5c3)c3ccc5c(c3)oc3ccccc35)oc3ccc5ccccc5c34)cc2)cc1 ; c1ccc(N(c2ccccc2)c2cc(N(c3ccc4ccccc4c3)c3ccc4c(c3)sc3ccccc34)c3oc4ccc5ccccc5c4c3c2)cc1 ; c1ccc(N(c2ccc3ccccc3c2)c2ccc3c(oc4ccc5ccccc5c43)c2N(c2ccc3ccccc3c2)c2ccc3sc4ccccc4c3c2)cc1] 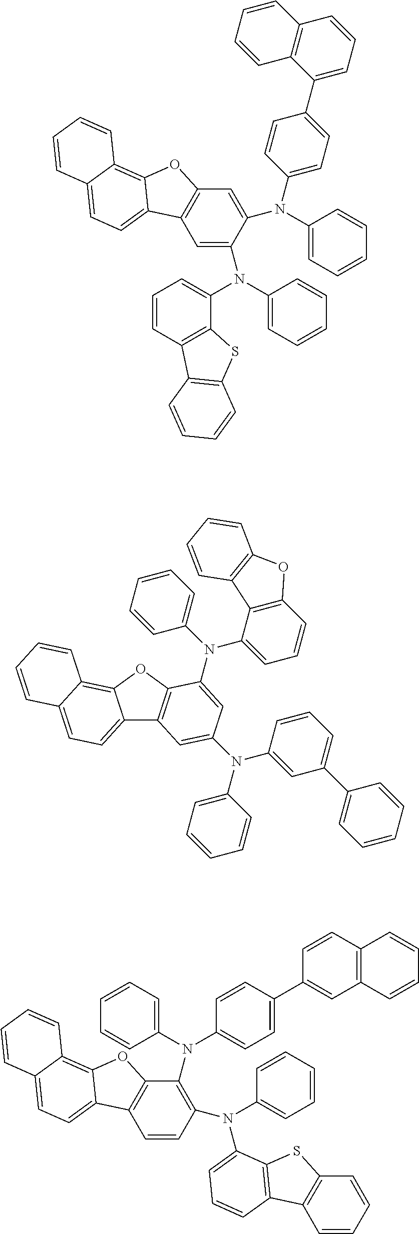 [SMILES: c1ccc(N(c2ccc(-c3cccc4ccccc34)cc2)c2cc3oc4c5ccccc5ccc4c3cc2N(c2ccccc2)c2cccc3c2sc2ccccc23)cc1 ; c1ccc(-c2cccc(N(c3ccccc3)c3cc(N(c4ccccc4)c4cccc5oc6ccccc6c45)c4oc5c6ccccc6ccc5c4c3)c2)cc1 ; c1ccc(N(c2ccc(-c3ccc4ccccc4c3)cc2)c2c(N(c3ccccc3)c3cccc4c3sc3ccccc34)ccc3c2oc2c4ccccc4ccc32)cc1] 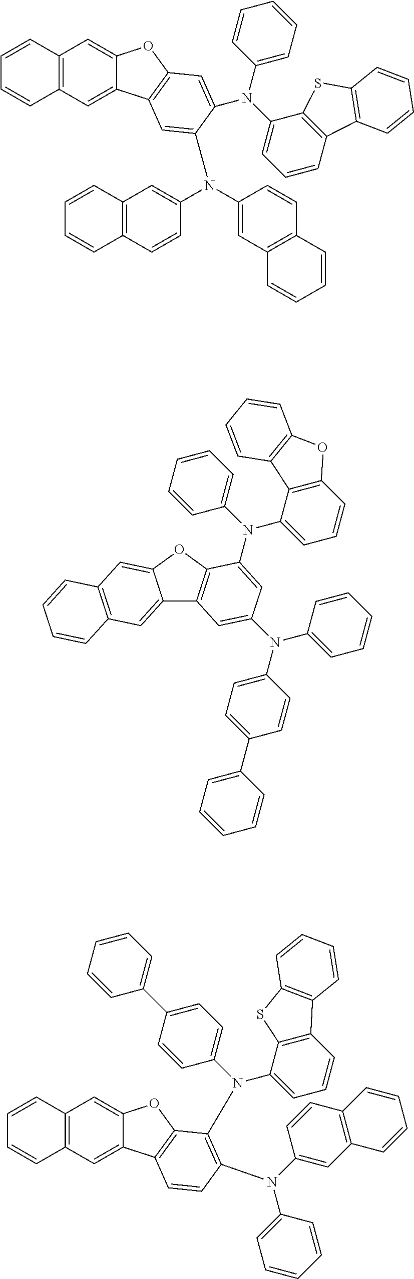 [SMILES: c1ccc(N(c2cc3oc4cc5ccccc5cc4c3cc2N(c2ccc3ccccc3c2)c2ccc3ccccc3c2)c2cccc3c2sc2ccccc23)cc1 ; c1ccc(-c2ccc(N(c3ccccc3)c3cc(N(c4ccccc4)c4cccc5oc6ccccc6c45)c4oc5cc6ccccc6cc5c4c3)cc2)cc1 ; c1ccc(-c2ccc(N(c3c(N(c4ccccc4)c4ccc5ccccc5c4)ccc4c3oc3cc5ccccc5cc34)c3cccc4c3sc3ccccc34)cc2)cc1] 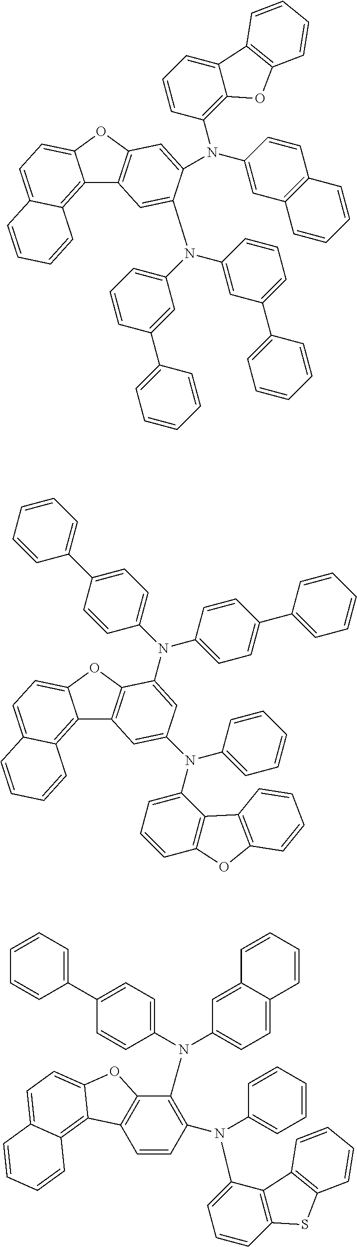 [SMILES: c1ccc(-c2cccc(N(c3cccc(-c4ccccc4)c3)c3cc4c(cc3N(c3ccc5ccccc5c3)c3cccc5c3oc3ccccc35)oc3ccc5ccccc5c34)c2)cc1 ; c1ccc(-c2ccc(N(c3ccc(-c4ccccc4)cc3)c3cc(N(c4ccccc4)c4cccc5oc6ccccc6c45)cc4c3oc3ccc5ccccc5c34)cc2)cc1 ; c1ccc(-c2ccc(N(c3ccc4ccccc4c3)c3c(N(c4ccccc4)c4cccc5sc6ccccc6c45)ccc4c3oc3ccc5ccccc5c34)cc2)cc1] 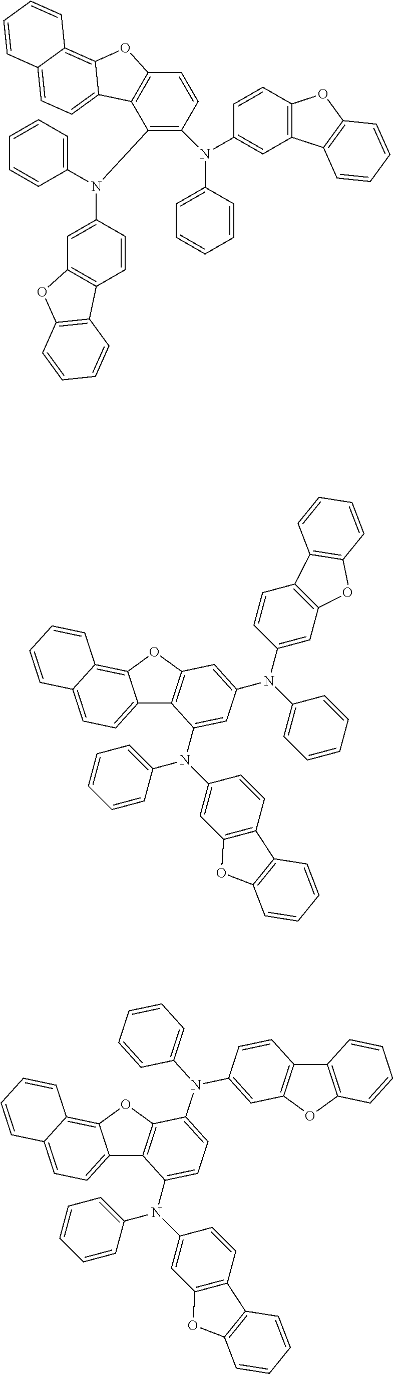 [SMILES: c1ccc(N(c2ccc3oc4ccccc4c3c2)c2ccc3oc4c5ccccc5ccc4c3c2N(c2ccccc2)c2ccc3c(c2)oc2ccccc23)cc1 ; c1ccc(N(c2ccc3c(c2)oc2ccccc23)c2cc(N(c3ccccc3)c3ccc4c(c3)oc3ccccc34)c3c(c2)oc2c4ccccc4ccc23)cc1 ; c1ccc(N(c2ccc3c(c2)oc2ccccc23)c2ccc(N(c3ccccc3)c3ccc4c(c3)oc3ccccc34)c3c2oc2c4ccccc4ccc23)cc1] 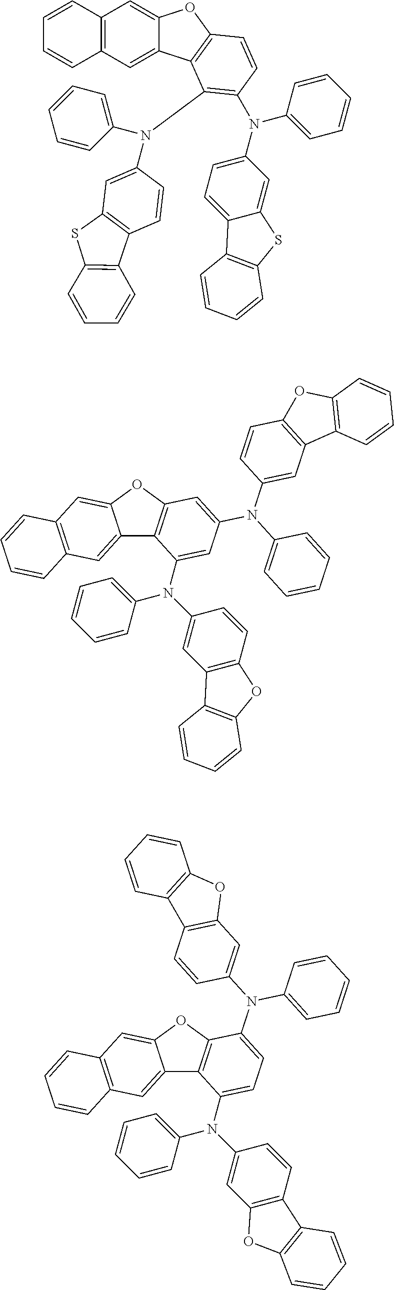 [SMILES: c1ccc(N(c2ccc3c(c2)sc2ccccc23)c2ccc3oc4cc5ccccc5cc4c3c2N(c2ccccc2)c2ccc3c(c2)sc2ccccc23)cc1 ; c1ccc(N(c2cc(N(c3ccccc3)c3ccc4oc5ccccc5c4c3)c3c(c2)oc2cc4ccccc4cc23)c2ccc3oc4ccccc4c3c2)cc1 ; c1ccc(N(c2ccc3c(c2)oc2ccccc23)c2ccc(N(c3ccccc3)c3ccc4c(c3)oc3ccccc34)c3c2oc2cc4ccccc4cc23)cc1] 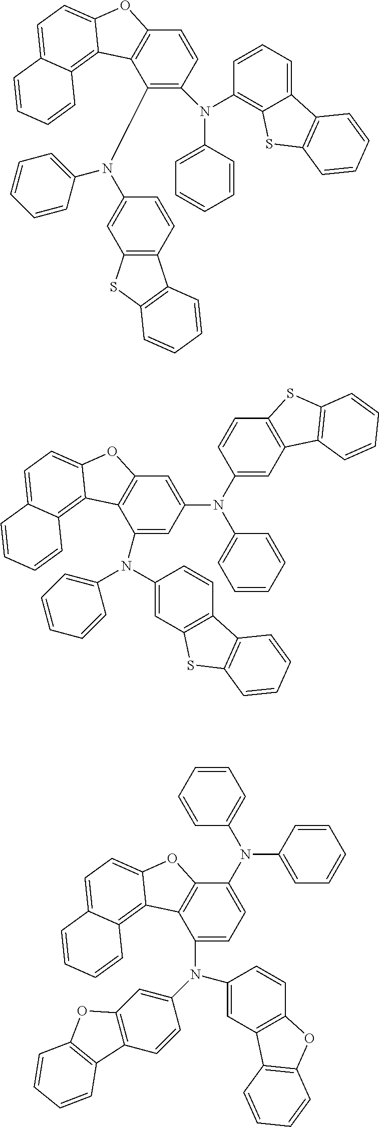 [SMILES: c1ccc(N(c2ccc3oc4ccc5ccccc5c4c3c2N(c2ccccc2)c2ccc3c(c2)sc2ccccc23)c2cccc3c2sc2ccccc23)cc1 ; c1ccc(N(c2cc(N(c3ccccc3)c3ccc4c(c3)sc3ccccc34)c3c(c2)oc2ccc4ccccc4c23)c2ccc3sc4ccccc4c3c2)cc1 ; c1ccc(N(c2ccccc2)c2ccc(N(c3ccc4c(c3)oc3ccccc34)c3ccc4oc5ccccc5c4c3)c3c2oc2ccc4ccccc4c23)cc1] 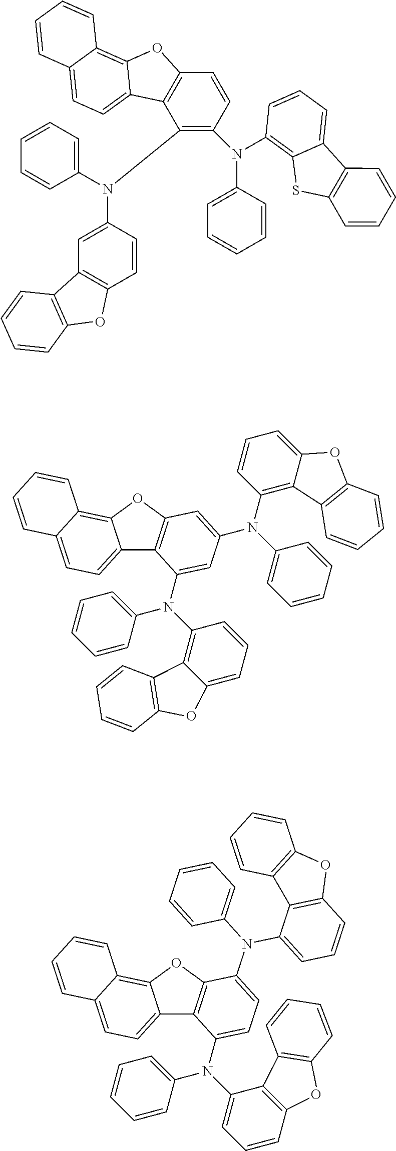 [SMILES: c1ccc(N(c2ccc3oc4c5ccccc5ccc4c3c2N(c2ccccc2)c2ccc3oc4ccccc4c3c2)c2cccc3c2sc2ccccc23)cc1 ; c1ccc(N(c2cc(N(c3ccccc3)c3cccc4oc5ccccc5c34)c3c(c2)oc2c4ccccc4ccc23)c2cccc3oc4ccccc4c23)cc1 ; c1ccc(N(c2ccc(N(c3ccccc3)c3cccc4oc5ccccc5c34)c3c2oc2c4ccccc4ccc23)c2cccc3oc4ccccc4c23)cc1] 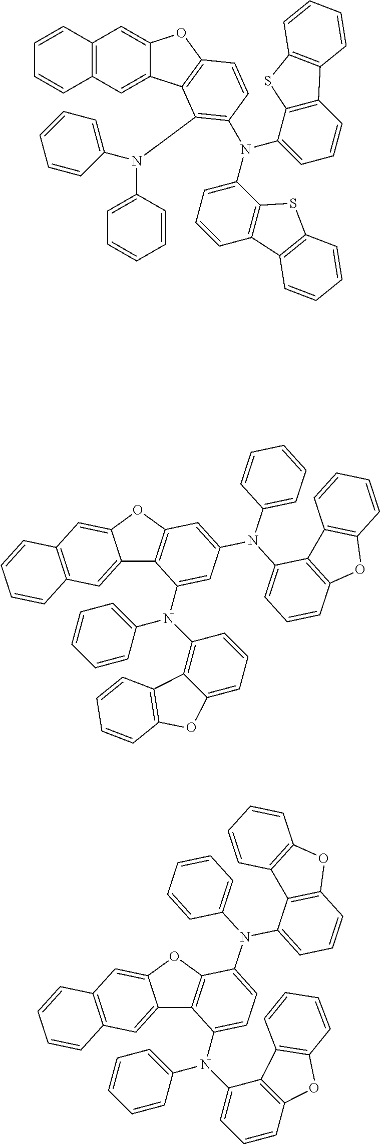 [SMILES: c1ccc(N(c2ccccc2)c2c(N(c3cccc4c3sc3ccccc34)c3cccc4c3sc3ccccc34)ccc3oc4cc5ccccc5cc4c23)cc1 ; c1ccc(N(c2cc(N(c3ccccc3)c3cccc4oc5ccccc5c34)c3c(c2)oc2cc4ccccc4cc23)c2cccc3oc4ccccc4c23)cc1 ; c1ccc(N(c2ccc(N(c3ccccc3)c3cccc4oc5ccccc5c34)c3c2oc2cc4ccccc4cc23)c2cccc3oc4ccccc4c23)cc1] 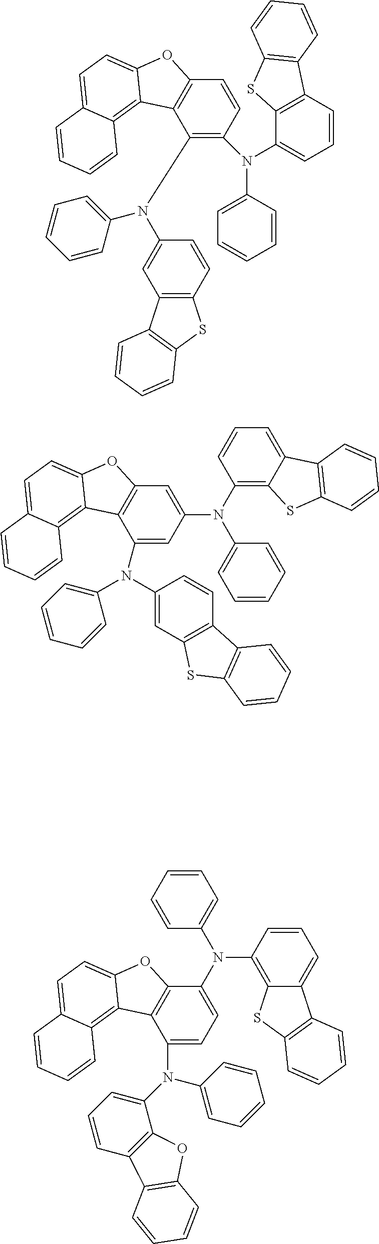 [SMILES: c1ccc(N(c2ccc3oc4ccc5ccccc5c4c3c2N(c2ccccc2)c2ccc3sc4ccccc4c3c2)c2cccc3c2sc2ccccc23)cc1 ; c1ccc(N(c2cc(N(c3ccccc3)c3ccc4c(c3)sc3ccccc34)c3c(c2)oc2ccc4ccccc4c23)c2cccc3c2sc2ccccc23)cc1 ; c1ccc(N(c2ccc(N(c3ccccc3)c3cccc4c3oc3ccccc34)c3c2oc2ccc4ccccc4c23)c2cccc3c2sc2ccccc23)cc1] 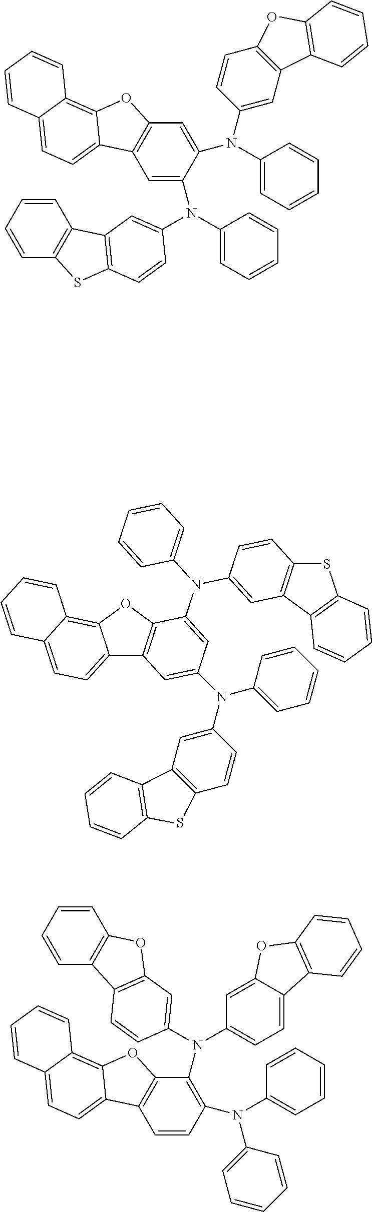 [SMILES: c1ccc(N(c2ccc3oc4ccccc4c3c2)c2cc3oc4c5ccccc5ccc4c3cc2N(c2ccccc2)c2ccc3sc4ccccc4c3c2)cc1 ; c1ccc(N(c2ccc3sc4ccccc4c3c2)c2cc(N(c3ccccc3)c3ccc4sc5ccccc5c4c3)c3oc4c5ccccc5ccc4c3c2)cc1 ; c1ccc(N(c2ccccc2)c2ccc3c(oc4c5ccccc5ccc34)c2N(c2ccc3c(c2)oc2ccccc23)c2ccc3c(c2)oc2ccccc23)cc1] 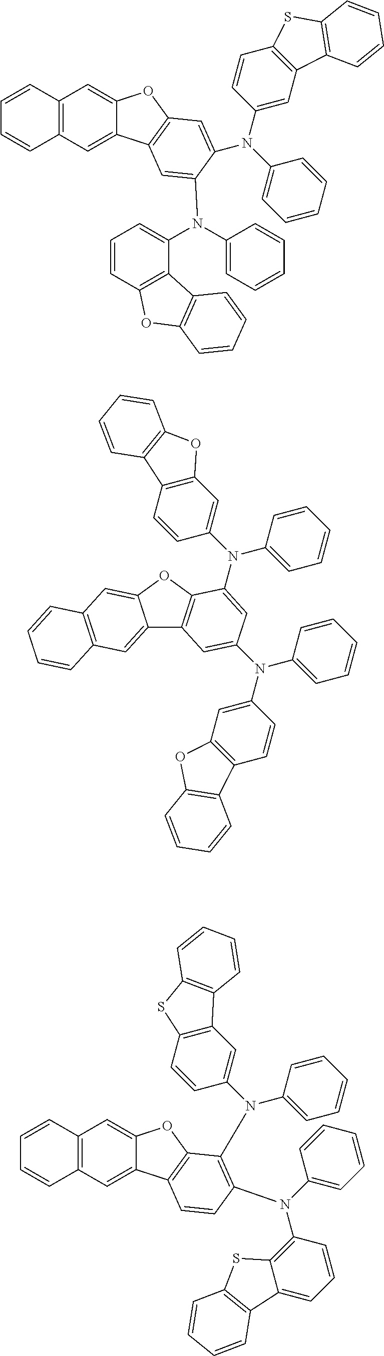 [SMILES: c1ccc(N(c2ccc3sc4ccccc4c3c2)c2cc3oc4cc5ccccc5cc4c3cc2N(c2ccccc2)c2cccc3oc4ccccc4c23)cc1 ; c1ccc(N(c2ccc3c(c2)oc2ccccc23)c2cc(N(c3ccccc3)c3ccc4c(c3)oc3ccccc34)c3oc4cc5ccccc5cc4c3c2)cc1 ; c1ccc(N(c2ccc3sc4ccccc4c3c2)c2c(N(c3ccccc3)c3cccc4c3sc3ccccc34)ccc3c2oc2cc4ccccc4cc23)cc1] 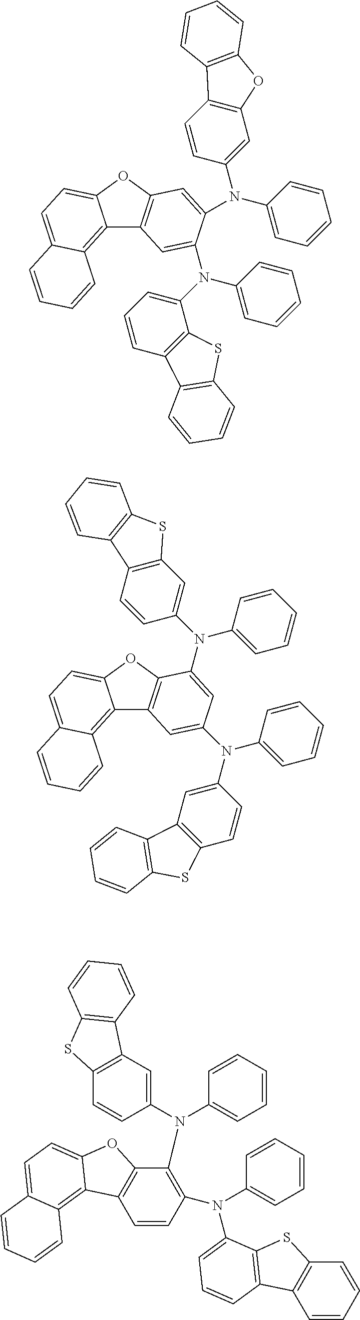 [SMILES: c1ccc(N(c2ccc3c(c2)oc2ccccc23)c2cc3oc4ccc5ccccc5c4c3cc2N(c2ccccc2)c2cccc3c2sc2ccccc23)cc1 ; c1ccc(N(c2ccc3sc4ccccc4c3c2)c2cc(N(c3ccccc3)c3ccc4c(c3)sc3ccccc34)c3oc4ccc5ccccc5c4c3c2)cc1 ; c1ccc(N(c2ccc3sc4ccccc4c3c2)c2c(N(c3ccccc3)c3cccc4c3sc3ccccc34)ccc3c2oc2ccc4ccccc4c23)cc1] 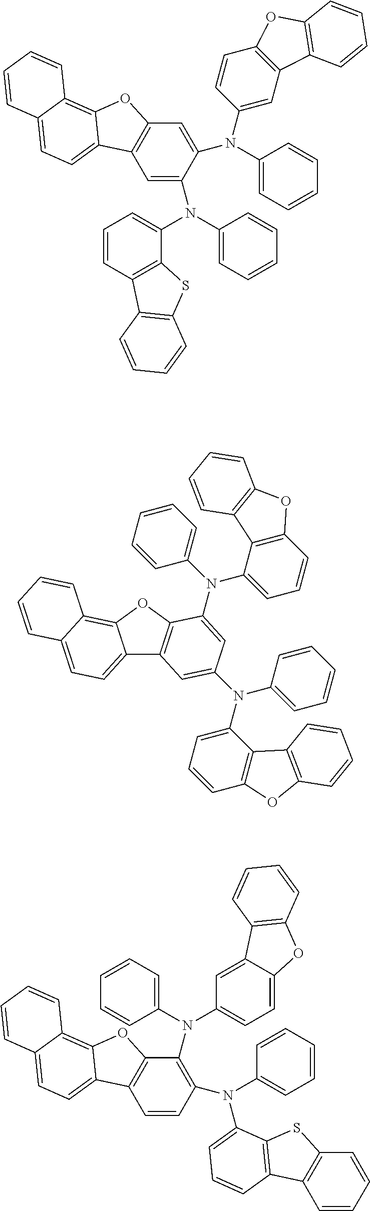 [SMILES: c1ccc(N(c2ccc3oc4ccccc4c3c2)c2cc3oc4c5ccccc5ccc4c3cc2N(c2ccccc2)c2cccc3c2sc2ccccc23)cc1 ; c1ccc(N(c2cc(N(c3ccccc3)c3cccc4oc5ccccc5c34)c3oc4c5ccccc5ccc4c3c2)c2cccc3oc4ccccc4c23)cc1 ; c1ccc(N(c2ccc3oc4ccccc4c3c2)c2c(N(c3ccccc3)c3cccc4c3sc3ccccc34)ccc3c2oc2c4ccccc4ccc32)cc1] 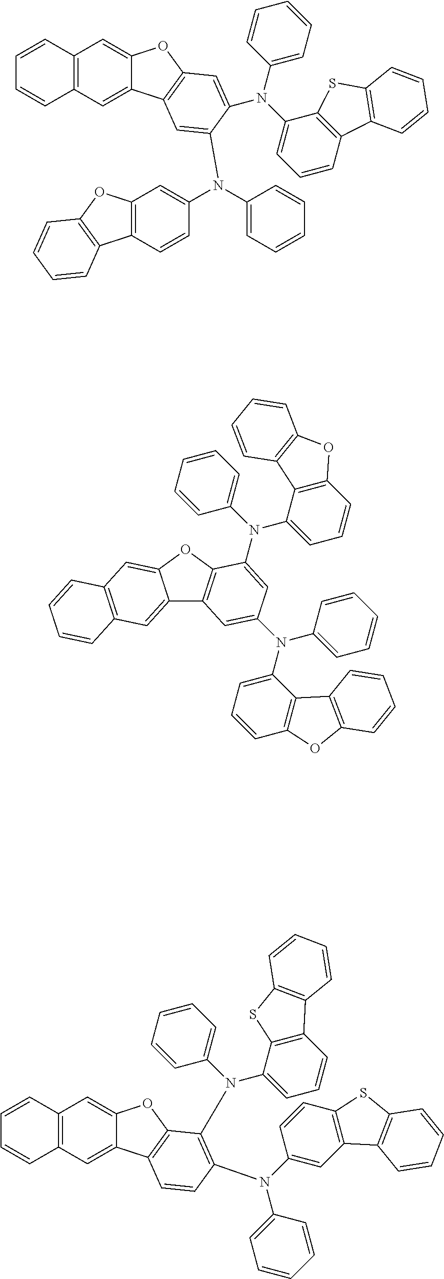 [SMILES: c1ccc(N(c2ccc3c(c2)oc2ccccc23)c2cc3c(cc2N(c2ccccc2)c2cccc4c2sc2ccccc24)oc2cc4ccccc4cc23)cc1 ; c1ccc(N(c2cc(N(c3ccccc3)c3cccc4oc5ccccc5c34)c3oc4cc5ccccc5cc4c3c2)c2cccc3oc4ccccc4c23)cc1 ; c1ccc(N(c2ccc3sc4ccccc4c3c2)c2ccc3c(oc4cc5ccccc5cc43)c2N(c2ccccc2)c2cccc3c2sc2ccccc23)cc1] 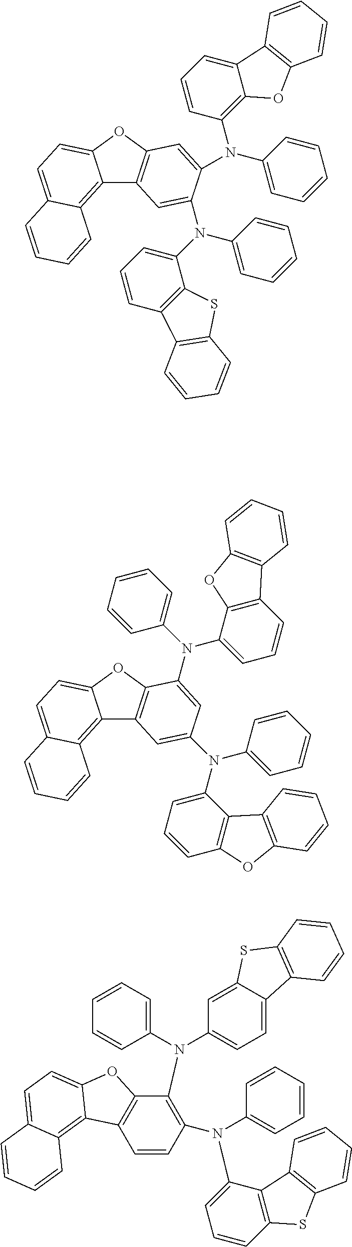 [SMILES: c1ccc(N(c2cc3oc4ccc5ccccc5c4c3cc2N(c2ccccc2)c2cccc3c2sc2ccccc23)c2cccc3c2oc2ccccc23)cc1 ; c1ccc(N(c2cccc3c2oc2ccccc23)c2cc(N(c3ccccc3)c3cccc4oc5ccccc5c34)cc3c2oc2ccc4ccccc4c23)cc1 ; c1ccc(N(c2ccc3c(c2)sc2ccccc23)c2c(N(c3ccccc3)c3cccc4sc5ccccc5c34)ccc3c2oc2ccc4ccccc4c23)cc1] 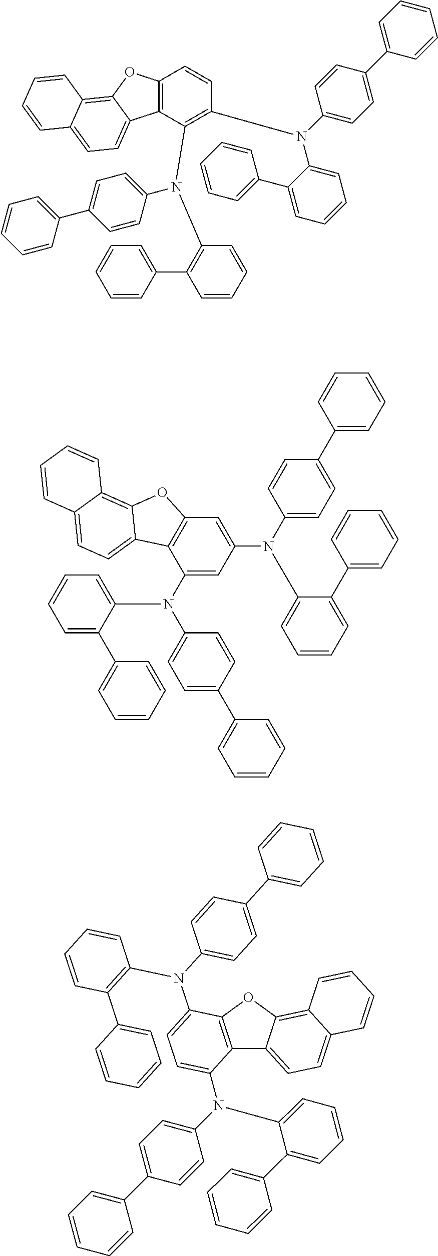 [SMILES: c1ccc(-c2ccc(N(c3ccccc3-c3ccccc3)c3ccc4oc5c6ccccc6ccc5c4c3N(c3ccc(-c4ccccc4)cc3)c3ccccc3-c3ccccc3)cc2)cc1 ; c1ccc(-c2ccc(N(c3cc(N(c4ccc(-c5ccccc5)cc4)c4ccccc4-c4ccccc4)c4c(c3)oc3c5ccccc5ccc34)c3ccccc3-c3ccccc3)cc2)cc1 ; c1ccc(-c2ccc(N(c3ccccc3-c3ccccc3)c3ccc(N(c4ccc(-c5ccccc5)cc4)c4ccccc4-c4ccccc4)c4c3oc3c5ccccc5ccc34)cc2)cc1] 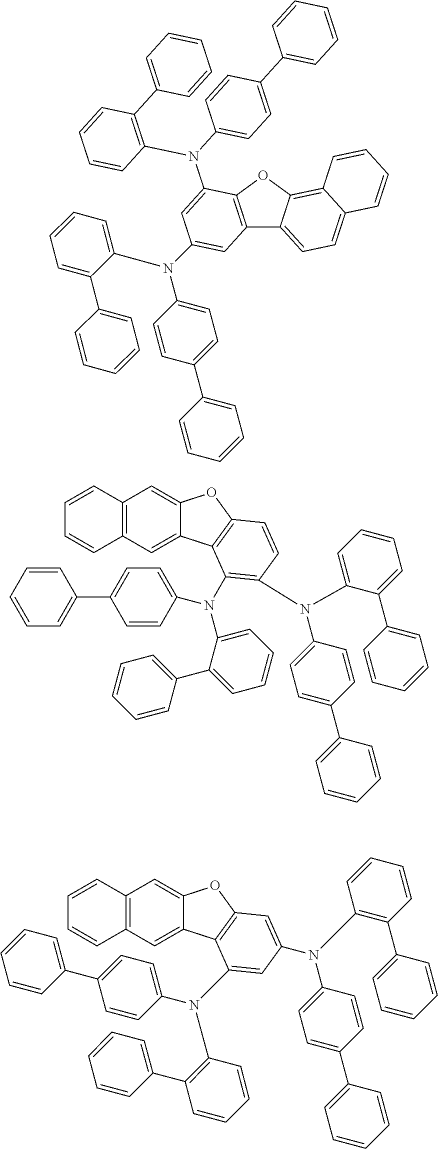 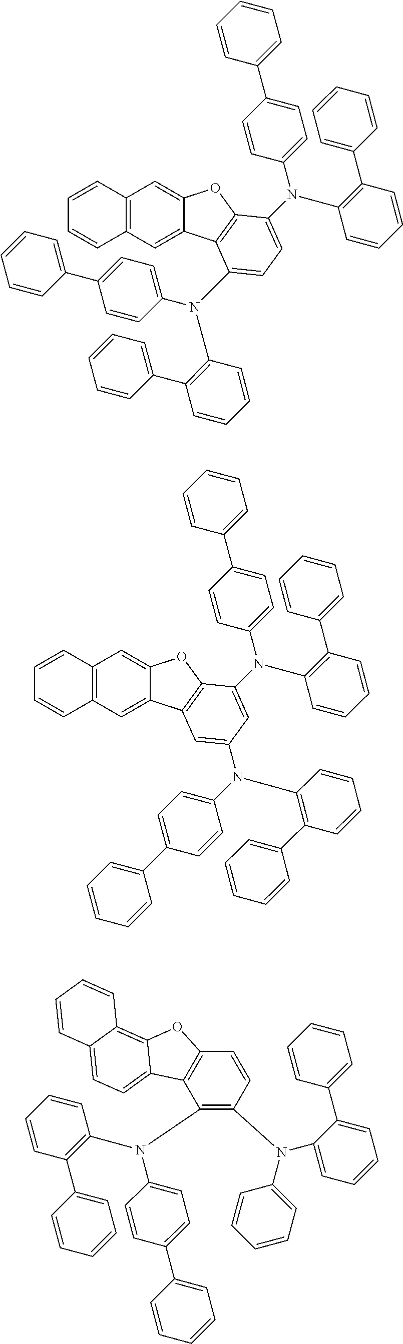 [SMILES: c1ccc(-c2ccc(N(c3ccccc3-c3ccccc3)c3ccc(N(c4ccc(-c5ccccc5)cc4)c4ccccc4-c4ccccc4)c4c3oc3cc5ccccc5cc34)cc2)cc1 ; c1ccc(-c2ccc(N(c3cc(N(c4ccc(-c5ccccc5)cc4)c4ccccc4-c4ccccc4)c4oc5cc6ccccc6cc5c4c3)c3ccccc3-c3ccccc3)cc2)cc1 ; c1ccc(-c2ccc(N(c3ccccc3-c3ccccc3)c3c(N(c4ccccc4)c4ccccc4-c4ccccc4)ccc4oc5c6ccccc6ccc5c34)cc2)cc1] 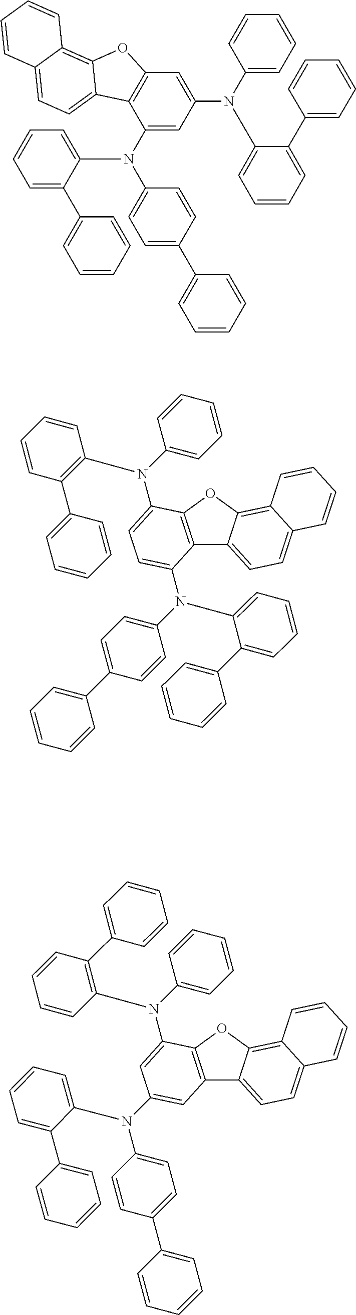 [SMILES: c1ccc(-c2ccc(N(c3ccccc3-c3ccccc3)c3cc(N(c4ccccc4)c4ccccc4-c4ccccc4)cc4oc5c6ccccc6ccc5c34)cc2)cc1 ; c1ccc(-c2ccc(N(c3ccccc3-c3ccccc3)c3ccc(N(c4ccccc4)c4ccccc4-c4ccccc4)c4oc5c6ccccc6ccc5c34)cc2)cc1 ; c1ccc(-c2ccc(N(c3cc(N(c4ccccc4)c4ccccc4-c4ccccc4)c4oc5c6ccccc6ccc5c4c3)c3ccccc3-c3ccccc3)cc2)cc1] 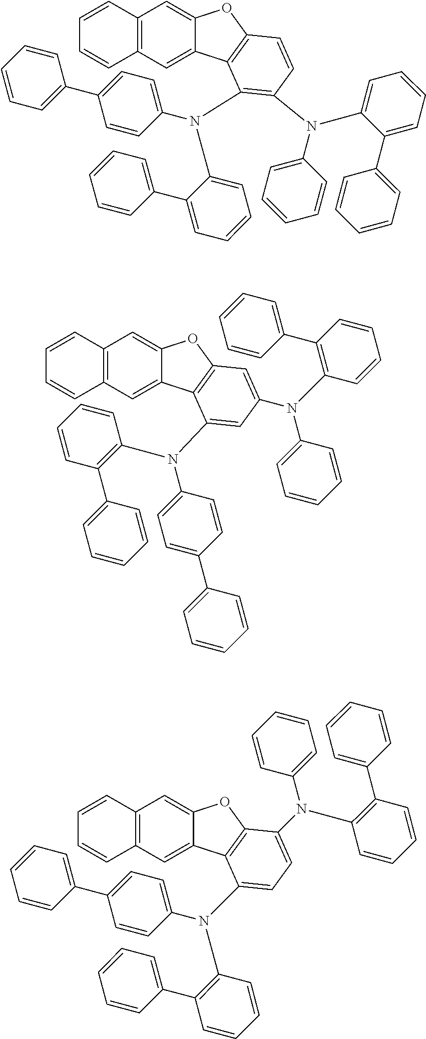 [SMILES: c1ccc(-c2ccc(N(c3ccccc3-c3ccccc3)c3c(N(c4ccccc4)c4ccccc4-c4ccccc4)ccc4oc5cc6ccccc6cc5c34)cc2)cc1 ; c1ccc(-c2ccc(N(c3ccccc3-c3ccccc3)c3cc(N(c4ccccc4)c4ccccc4-c4ccccc4)cc4oc5cc6ccccc6cc5c34)cc2)cc1 ; c1ccc(-c2ccc(N(c3ccccc3-c3ccccc3)c3ccc(N(c4ccccc4)c4ccccc4-c4ccccc4)c4oc5cc6ccccc6cc5c34)cc2)cc1] 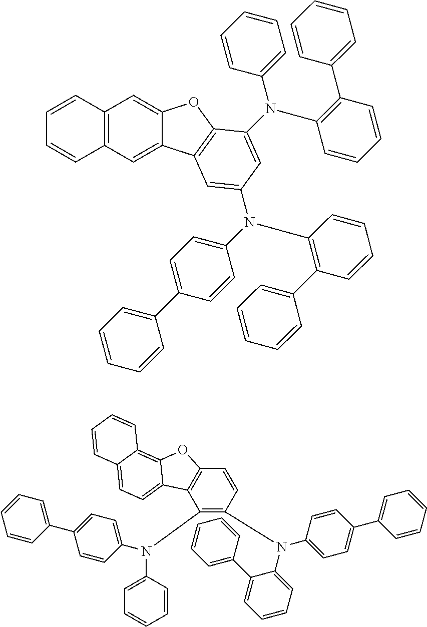 [SMILES: c1ccc(-c2ccc(N(c3cc(N(c4ccccc4)c4ccccc4-c4ccccc4)c4oc5cc6ccccc6cc5c4c3)c3ccccc3-c3ccccc3)cc2)cc1 ; c1ccc(-c2ccc(N(c3ccccc3-c3ccccc3)c3ccc4oc5c6ccccc6ccc5c4c3N(c3ccccc3)c3ccc(-c4ccccc4)cc3)cc2)cc1] 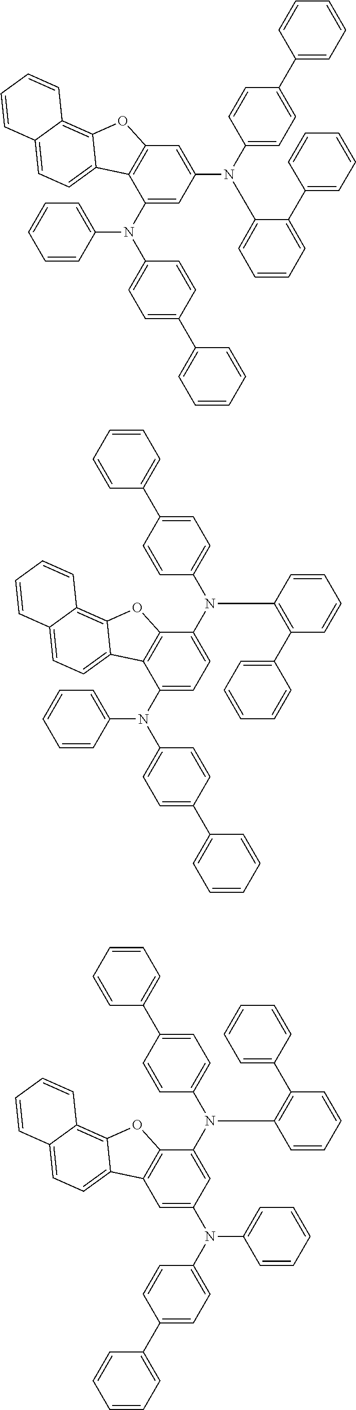 [SMILES: c1ccc(-c2ccc(N(c3cc(N(c4ccccc4)c4ccc(-c5ccccc5)cc4)c4c(c3)oc3c5ccccc5ccc34)c3ccccc3-c3ccccc3)cc2)cc1 ; c1ccc(-c2ccc(N(c3ccccc3-c3ccccc3)c3ccc(N(c4ccccc4)c4ccc(-c5ccccc5)cc4)c4c3oc3c5ccccc5ccc34)cc2)cc1 ; c1ccc(-c2ccc(N(c3ccccc3)c3cc(N(c4ccc(-c5ccccc5)cc4)c4ccccc4-c4ccccc4)c4oc5c6ccccc6ccc5c4c3)cc2)cc1] 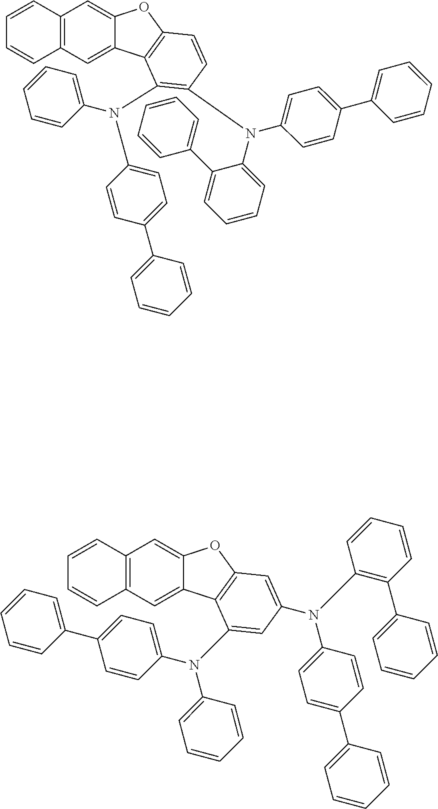 [SMILES: c1ccc(-c2ccc(N(c3ccccc3-c3ccccc3)c3ccc4oc5cc6ccccc6cc5c4c3N(c3ccccc3)c3ccc(-c4ccccc4)cc3)cc2)cc1 ; c1ccc(-c2ccc(N(c3cc(N(c4ccccc4)c4ccc(-c5ccccc5)cc4)c4c(c3)oc3cc5ccccc5cc34)c3ccccc3-c3ccccc3)cc2)cc1] 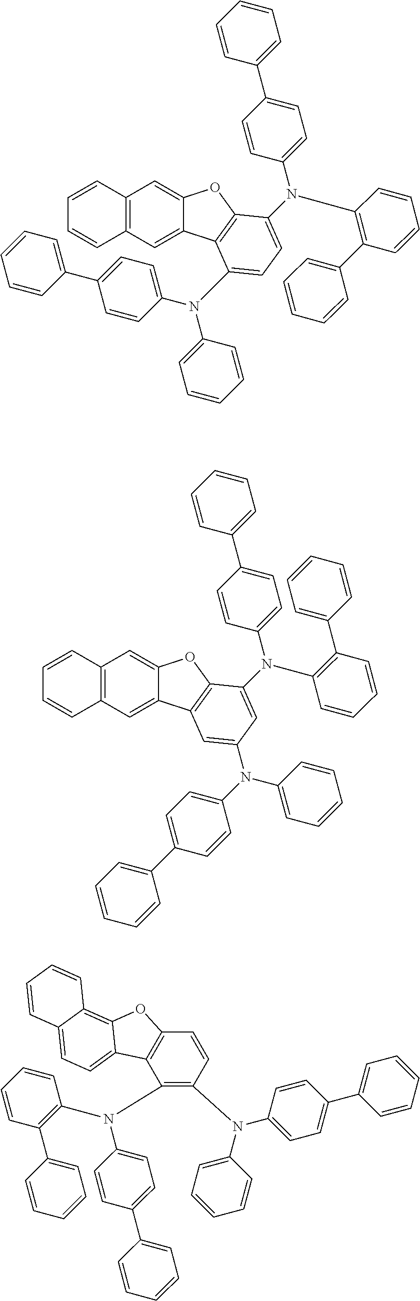 [SMILES: c1ccc(-c2ccc(N(c3ccccc3-c3ccccc3)c3ccc(N(c4ccccc4)c4ccc(-c5ccccc5)cc4)c4c3oc3cc5ccccc5cc34)cc2)cc1 ; c1ccc(-c2ccc(N(c3ccccc3)c3cc(N(c4ccc(-c5ccccc5)cc4)c4ccccc4-c4ccccc4)c4oc5cc6ccccc6cc5c4c3)cc2)cc1 ; c1ccc(-c2ccc(N(c3ccccc3)c3ccc4oc5c6ccccc6ccc5c4c3N(c3ccc(-c4ccccc4)cc3)c3ccccc3-c3ccccc3)cc2)cc1] 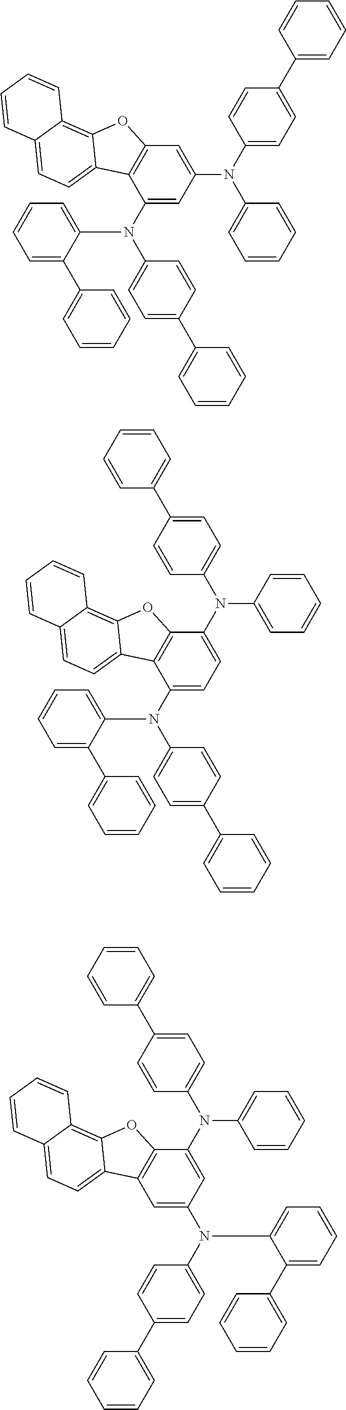 [SMILES: c1ccc(-c2ccc(N(c3ccccc3)c3cc(N(c4ccc(-c5ccccc5)cc4)c4ccccc4-c4ccccc4)c4c(c3)oc3c5ccccc5ccc34)cc2)cc1 ; c1ccc(-c2ccc(N(c3ccccc3)c3ccc(N(c4ccc(-c5ccccc5)cc4)c4ccccc4-c4ccccc4)c4c3oc3c5ccccc5ccc34)cc2)cc1 ; c1ccc(-c2ccc(N(c3cc(N(c4ccccc4)c4ccc(-c5ccccc5)cc4)c4oc5c6ccccc6ccc5c4c3)c3ccccc3-c3ccccc3)cc2)cc1] 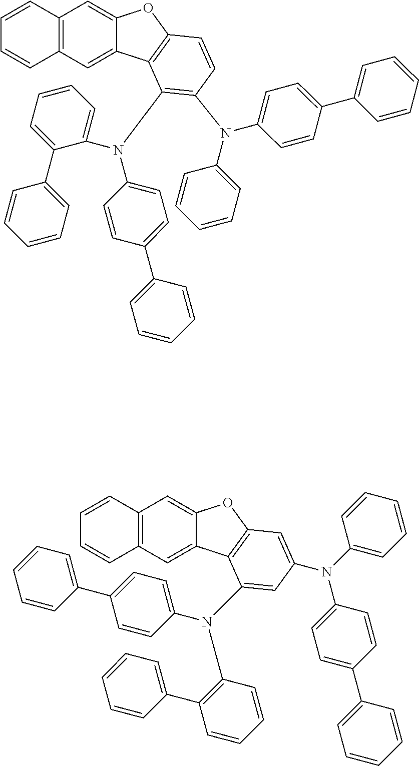 [SMILES: c1ccc(-c2ccc(N(c3ccccc3)c3ccc4oc5cc6ccccc6cc5c4c3N(c3ccc(-c4ccccc4)cc3)c3ccccc3-c3ccccc3)cc2)cc1 ; c1ccc(-c2ccc(N(c3ccccc3)c3cc(N(c4ccc(-c5ccccc5)cc4)c4ccccc4-c4ccccc4)c4c(c3)oc3cc5ccccc5cc34)cc2)cc1] 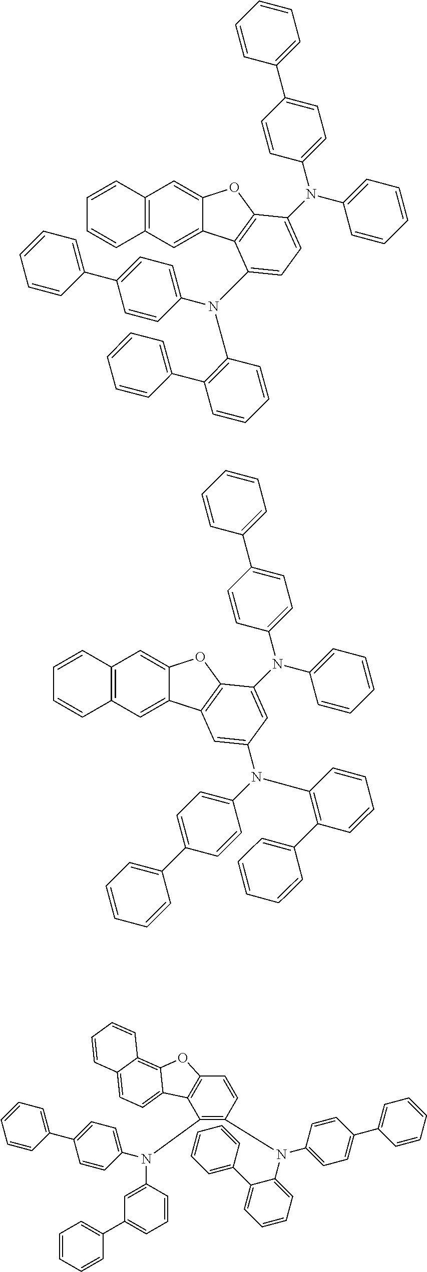 [SMILES: c1ccc(-c2ccc(N(c3ccccc3)c3ccc(N(c4ccc(-c5ccccc5)cc4)c4ccccc4-c4ccccc4)c4c3oc3cc5ccccc5cc34)cc2)cc1 ; c1ccc(-c2ccc(N(c3cc(N(c4ccccc4)c4ccc(-c5ccccc5)cc4)c4oc5cc6ccccc6cc5c4c3)c3ccccc3-c3ccccc3)cc2)cc1 ; c1ccc(-c2ccc(N(c3ccccc3-c3ccccc3)c3ccc4oc5c6ccccc6ccc5c4c3N(c3ccc(-c4ccccc4)cc3)c3cccc(-c4ccccc4)c3)cc2)cc1] 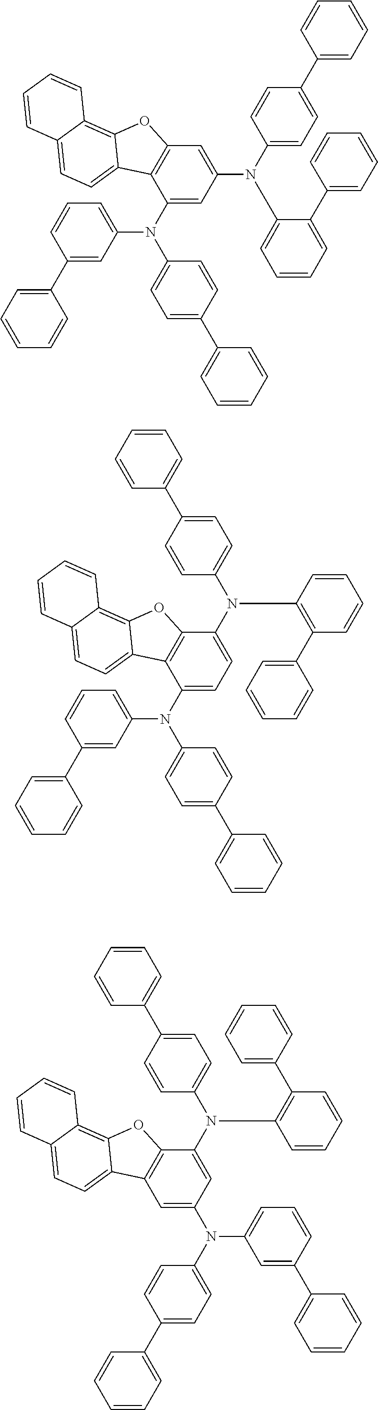 [SMILES: c1ccc(-c2ccc(N(c3cc(N(c4ccc(-c5ccccc5)cc4)c4cccc(-c5ccccc5)c4)c4c(c3)oc3c5ccccc5ccc34)c3ccccc3-c3ccccc3)cc2)cc1 ; c1ccc(-c2ccc(N(c3ccccc3-c3ccccc3)c3ccc(N(c4ccc(-c5ccccc5)cc4)c4cccc(-c5ccccc5)c4)c4c3oc3c5ccccc5ccc34)cc2)cc1 ; c1ccc(-c2ccc(N(c3cccc(-c4ccccc4)c3)c3cc(N(c4ccc(-c5ccccc5)cc4)c4ccccc4-c4ccccc4)c4oc5c6ccccc6ccc5c4c3)cc2)cc1] 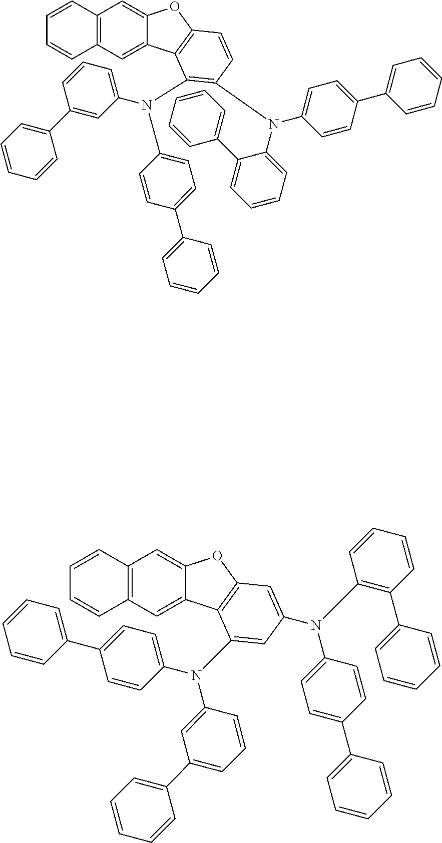 [SMILES: c1ccc(-c2ccc(N(c3ccccc3-c3ccccc3)c3ccc4oc5cc6ccccc6cc5c4c3N(c3ccc(-c4ccccc4)cc3)c3cccc(-c4ccccc4)c3)cc2)cc1 ; c1ccc(-c2ccc(N(c3cc(N(c4ccc(-c5ccccc5)cc4)c4cccc(-c5ccccc5)c4)c4c(c3)oc3cc5ccccc5cc34)c3ccccc3-c3ccccc3)cc2)cc1] 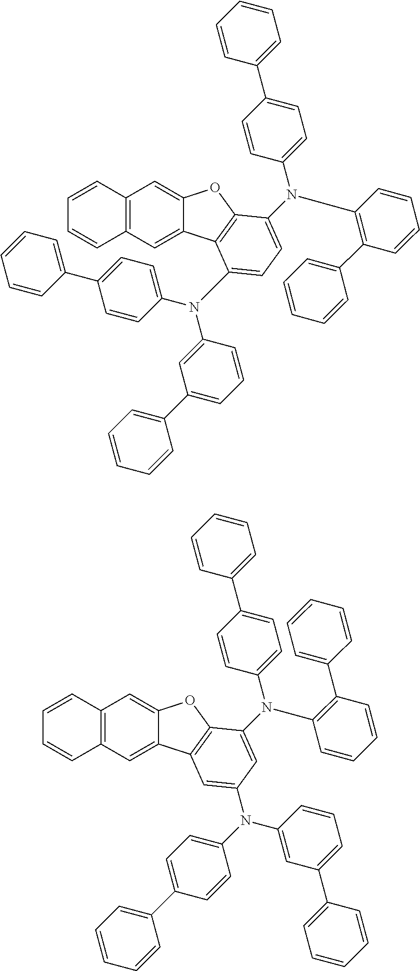 [SMILES: c1ccc(-c2ccc(N(c3ccccc3-c3ccccc3)c3ccc(N(c4ccc(-c5ccccc5)cc4)c4cccc(-c5ccccc5)c4)c4c3oc3cc5ccccc5cc34)cc2)cc1 ; c1ccc(-c2ccc(N(c3cccc(-c4ccccc4)c3)c3cc(N(c4ccc(-c5ccccc5)cc4)c4ccccc4-c4ccccc4)c4oc5cc6ccccc6cc5c4c3)cc2)cc1] 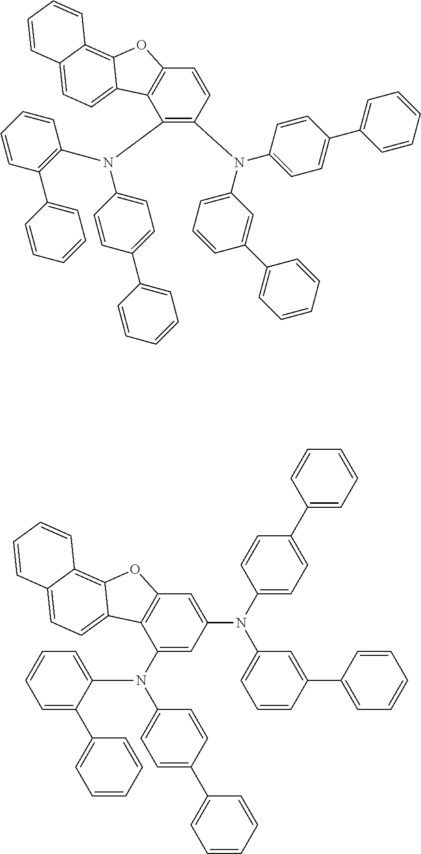 [SMILES: c1ccc(-c2ccc(N(c3cccc(-c4ccccc4)c3)c3ccc4oc5c6ccccc6ccc5c4c3N(c3ccc(-c4ccccc4)cc3)c3ccccc3-c3ccccc3)cc2)cc1 ; c1ccc(-c2ccc(N(c3cccc(-c4ccccc4)c3)c3cc(N(c4ccc(-c5ccccc5)cc4)c4ccccc4-c4ccccc4)c4c(c3)oc3c5ccccc5ccc34)cc2)cc1] 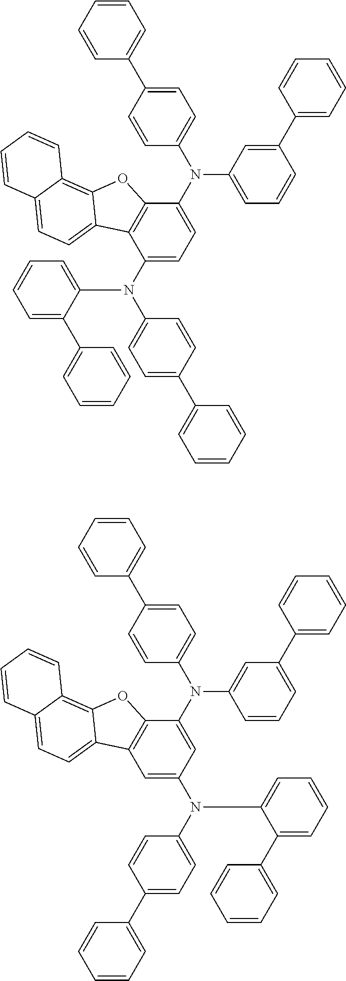 [SMILES: c1ccc(-c2ccc(N(c3cccc(-c4ccccc4)c3)c3ccc(N(c4ccc(-c5ccccc5)cc4)c4ccccc4-c4ccccc4)c4c3oc3c5ccccc5ccc34)cc2)cc1 ; c1ccc(-c2ccc(N(c3cc(N(c4ccc(-c5ccccc5)cc4)c4cccc(-c5ccccc5)c4)c4oc5c6ccccc6ccc5c4c3)c3ccccc3-c3ccccc3)cc2)cc1] 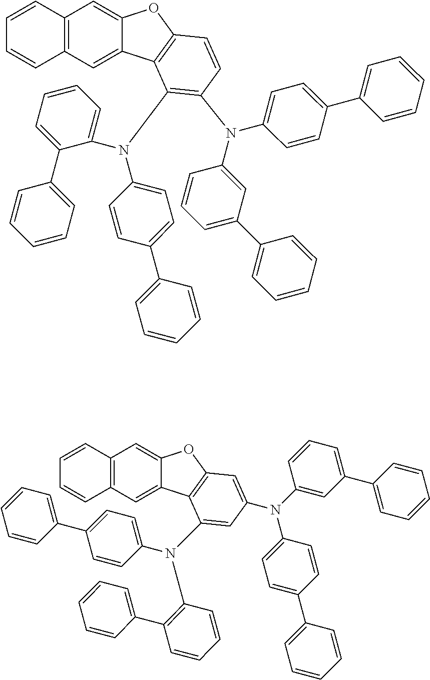 [SMILES: c1ccc(-c2ccc(N(c3cccc(-c4ccccc4)c3)c3ccc4oc5cc6ccccc6cc5c4c3N(c3ccc(-c4ccccc4)cc3)c3ccccc3-c3ccccc3)cc2)cc1 ; c1ccc(-c2ccc(N(c3cccc(-c4ccccc4)c3)c3cc(N(c4ccc(-c5ccccc5)cc4)c4ccccc4-c4ccccc4)c4c(c3)oc3cc5ccccc5cc34)cc2)cc1] 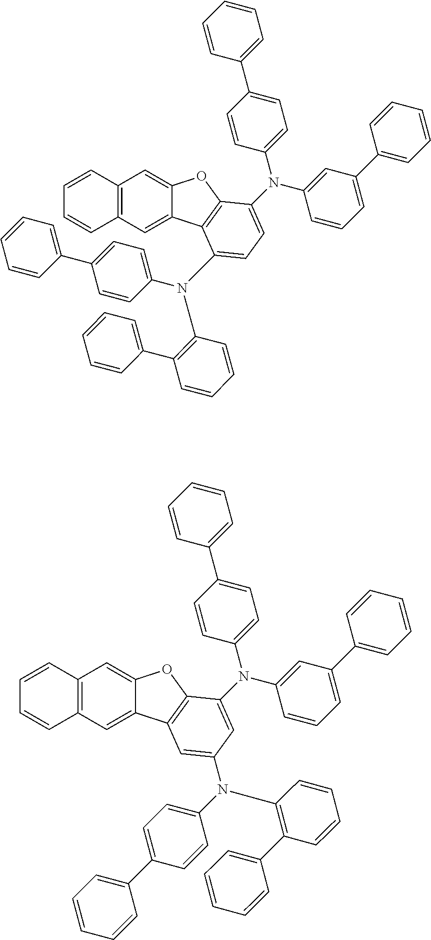 [SMILES: c1ccc(-c2ccc(N(c3cccc(-c4ccccc4)c3)c3ccc(N(c4ccc(-c5ccccc5)cc4)c4ccccc4-c4ccccc4)c4c3oc3cc5ccccc5cc34)cc2)cc1 ; c1ccc(-c2ccc(N(c3cc(N(c4ccc(-c5ccccc5)cc4)c4cccc(-c5ccccc5)c4)c4oc5cc6ccccc6cc5c4c3)c3ccccc3-c3ccccc3)cc2)cc1] 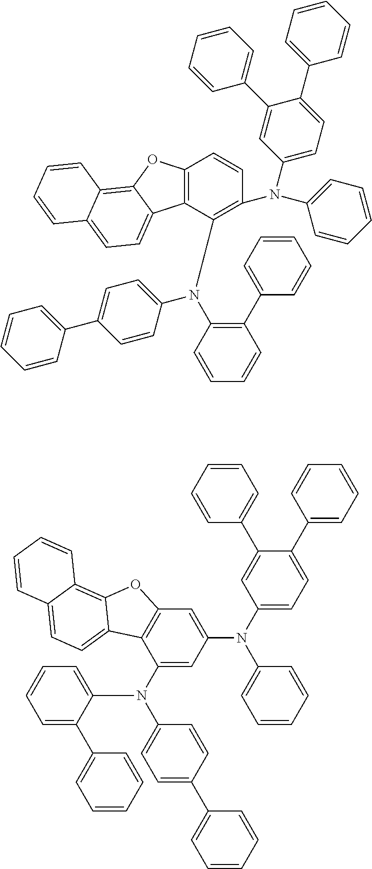 [SMILES: c1ccc(-c2ccc(N(c3ccccc3-c3ccccc3)c3c(N(c4ccccc4)c4ccc(-c5ccccc5)c(-c5ccccc5)c4)ccc4oc5c6ccccc6ccc5c34)cc2)cc1 ; c1ccc(-c2ccc(N(c3ccccc3-c3ccccc3)c3cc(N(c4ccccc4)c4ccc(-c5ccccc5)c(-c5ccccc5)c4)cc4oc5c6ccccc6ccc5c34)cc2)cc1] 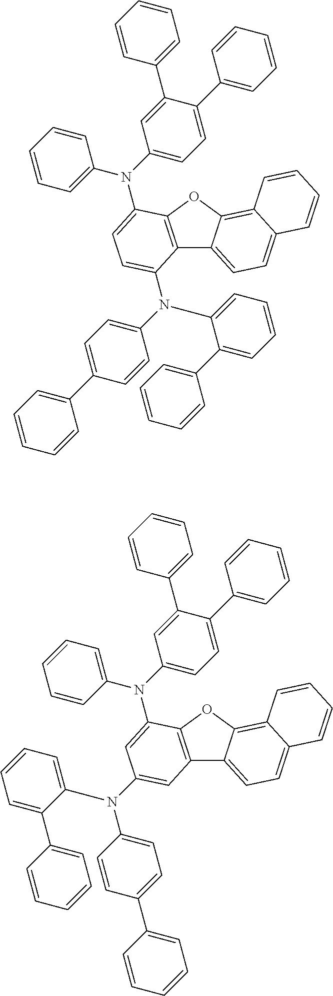 [SMILES: c1ccc(-c2ccc(N(c3ccccc3-c3ccccc3)c3ccc(N(c4ccccc4)c4ccc(-c5ccccc5)c(-c5ccccc5)c4)c4oc5c6ccccc6ccc5c34)cc2)cc1 ; c1ccc(-c2ccc(N(c3cc(N(c4ccccc4)c4ccc(-c5ccccc5)c(-c5ccccc5)c4)c4oc5c6ccccc6ccc5c4c3)c3ccccc3-c3ccccc3)cc2)cc1] 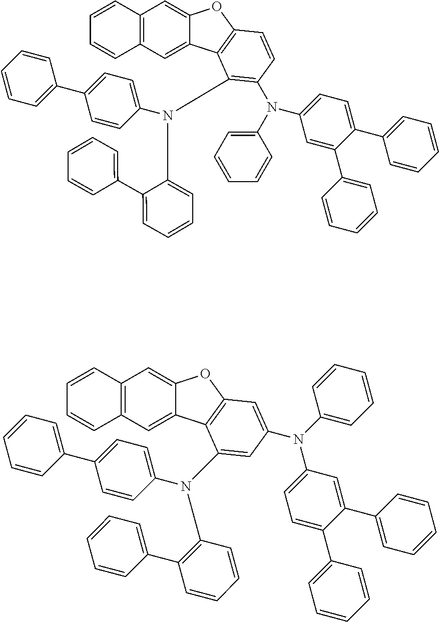 [SMILES: c1ccc(-c2ccc(N(c3ccccc3-c3ccccc3)c3c(N(c4ccccc4)c4ccc(-c5ccccc5)c(-c5ccccc5)c4)ccc4oc5cc6ccccc6cc5c34)cc2)cc1 ; c1ccc(-c2ccc(N(c3ccccc3-c3ccccc3)c3cc(N(c4ccccc4)c4ccc(-c5ccccc5)c(-c5ccccc5)c4)cc4oc5cc6ccccc6cc5c34)cc2)cc1] 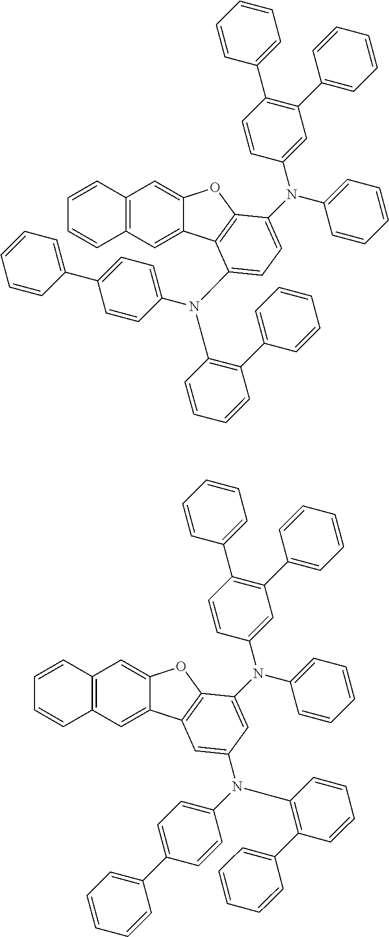 [SMILES: c1ccc(-c2ccc(N(c3ccccc3-c3ccccc3)c3ccc(N(c4ccccc4)c4ccc(-c5ccccc5)c(-c5ccccc5)c4)c4oc5cc6ccccc6cc5c34)cc2)cc1 ; c1ccc(-c2ccc(N(c3cc(N(c4ccccc4)c4ccc(-c5ccccc5)c(-c5ccccc5)c4)c4oc5cc6ccccc6cc5c4c3)c3ccccc3-c3ccccc3)cc2)cc1] 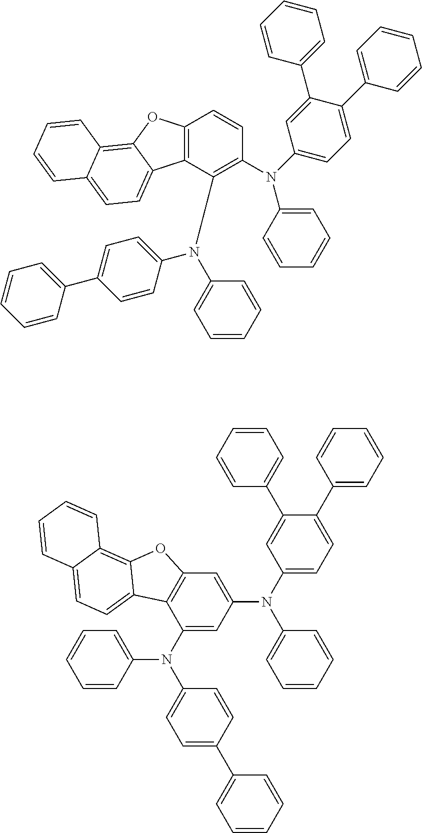 [SMILES: c1ccc(-c2ccc(N(c3ccccc3)c3c(N(c4ccccc4)c4ccc(-c5ccccc5)c(-c5ccccc5)c4)ccc4oc5c6ccccc6ccc5c34)cc2)cc1 ; c1ccc(-c2ccc(N(c3ccccc3)c3cc(N(c4ccccc4)c4ccc(-c5ccccc5)c(-c5ccccc5)c4)cc4oc5c6ccccc6ccc5c34)cc2)cc1] 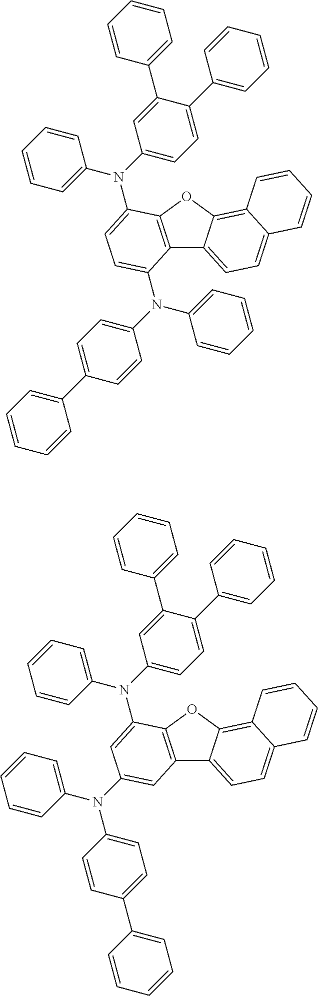 [SMILES: c1ccc(-c2ccc(N(c3ccccc3)c3ccc(N(c4ccccc4)c4ccc(-c5ccccc5)c(-c5ccccc5)c4)c4oc5c6ccccc6ccc5c34)cc2)cc1 ; c1ccc(-c2ccc(N(c3ccccc3)c3cc(N(c4ccccc4)c4ccc(-c5ccccc5)c(-c5ccccc5)c4)c4oc5c6ccccc6ccc5c4c3)cc2)cc1] 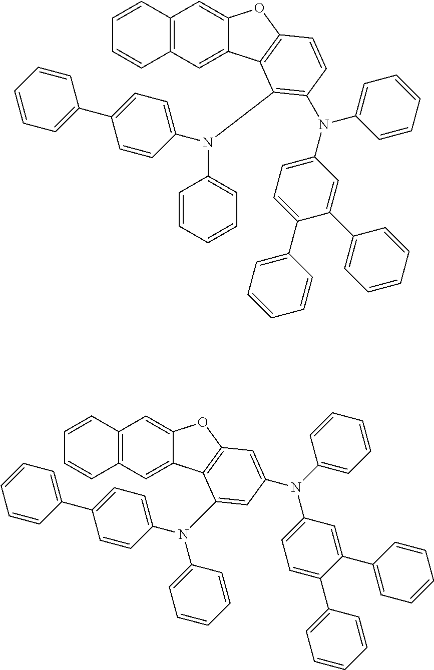 [SMILES: c1ccc(-c2ccc(N(c3ccccc3)c3c(N(c4ccccc4)c4ccc(-c5ccccc5)c(-c5ccccc5)c4)ccc4oc5cc6ccccc6cc5c34)cc2)cc1 ; c1ccc(-c2ccc(N(c3ccccc3)c3cc(N(c4ccccc4)c4ccc(-c5ccccc5)c(-c5ccccc5)c4)cc4oc5cc6ccccc6cc5c34)cc2)cc1] 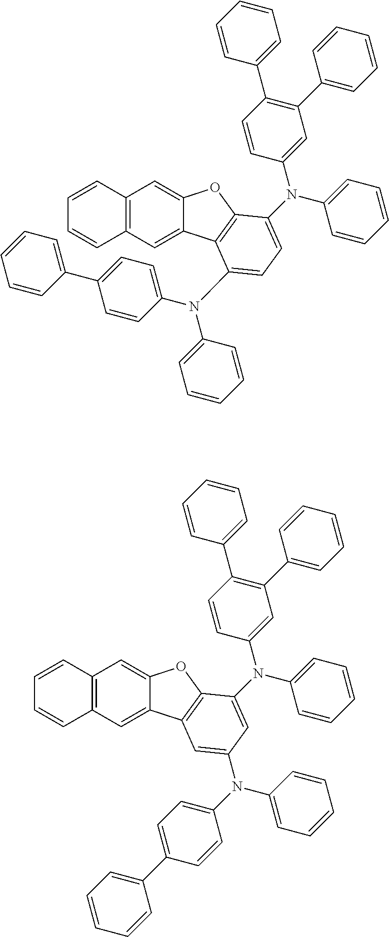 [SMILES: c1ccc(-c2ccc(N(c3ccccc3)c3ccc(N(c4ccccc4)c4ccc(-c5ccccc5)c(-c5ccccc5)c4)c4oc5cc6ccccc6cc5c34)cc2)cc1 ; c1ccc(-c2ccc(N(c3ccccc3)c3cc(N(c4ccccc4)c4ccc(-c5ccccc5)c(-c5ccccc5)c4)c4oc5cc6ccccc6cc5c4c3)cc2)cc1] 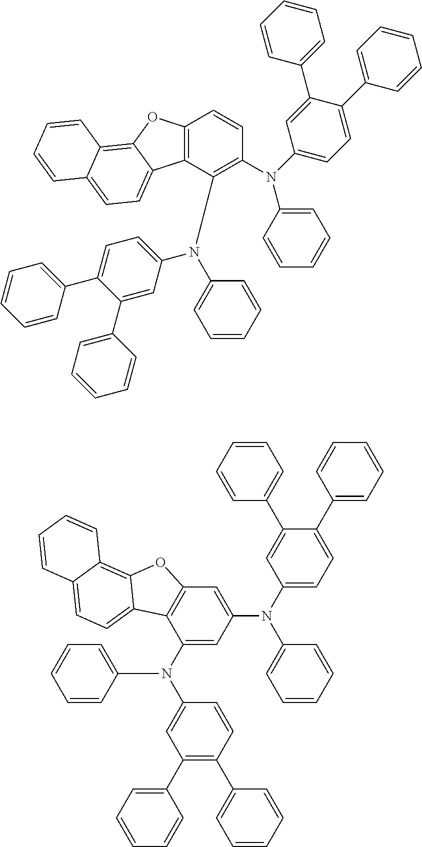 [SMILES: c1ccc(-c2ccc(N(c3ccccc3)c3ccc4oc5c6ccccc6ccc5c4c3N(c3ccccc3)c3ccc(-c4ccccc4)c(-c4ccccc4)c3)cc2-c2ccccc2)cc1 ; c1ccc(-c2ccc(N(c3ccccc3)c3cc(N(c4ccccc4)c4ccc(-c5ccccc5)c(-c5ccccc5)c4)c4c(c3)oc3c5ccccc5ccc34)cc2-c2ccccc2)cc1] 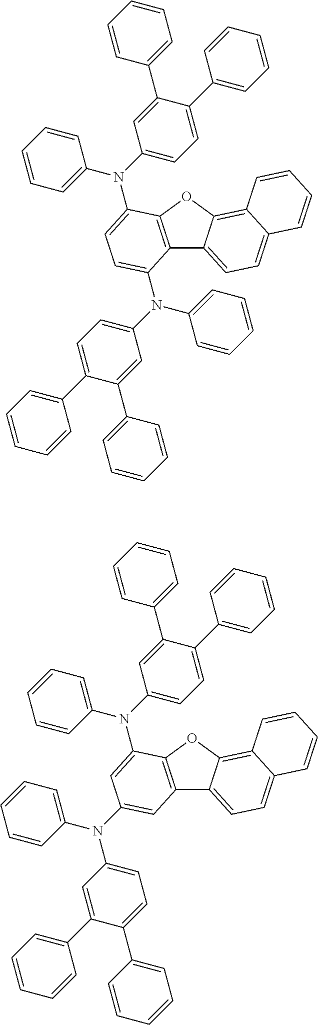 [SMILES: c1ccc(-c2ccc(N(c3ccccc3)c3ccc(N(c4ccccc4)c4ccc(-c5ccccc5)c(-c5ccccc5)c4)c4c3oc3c5ccccc5ccc34)cc2-c2ccccc2)cc1 ; c1ccc(-c2ccc(N(c3ccccc3)c3cc(N(c4ccccc4)c4ccc(-c5ccccc5)c(-c5ccccc5)c4)c4oc5c6ccccc6ccc5c4c3)cc2-c2ccccc2)cc1] 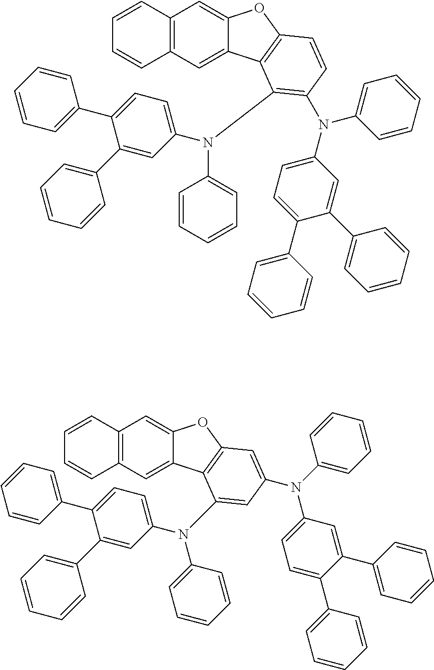 [SMILES: c1ccc(-c2ccc(N(c3ccccc3)c3ccc4oc5cc6ccccc6cc5c4c3N(c3ccccc3)c3ccc(-c4ccccc4)c(-c4ccccc4)c3)cc2-c2ccccc2)cc1 ; c1ccc(-c2ccc(N(c3ccccc3)c3cc(N(c4ccccc4)c4ccc(-c5ccccc5)c(-c5ccccc5)c4)c4c(c3)oc3cc5ccccc5cc34)cc2-c2ccccc2)cc1] 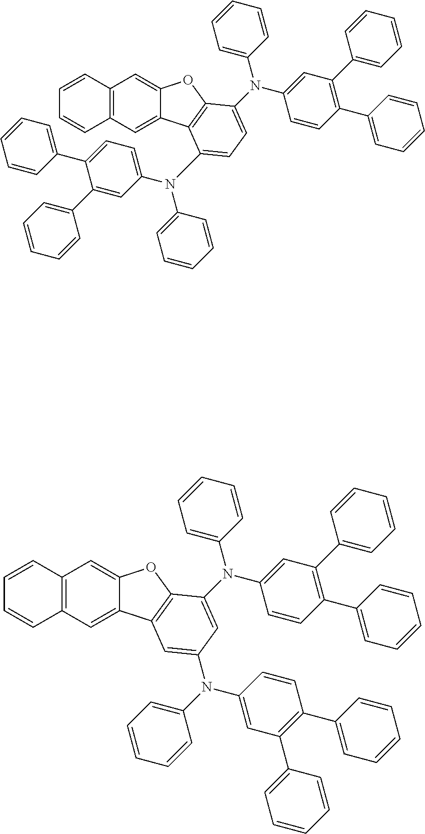 [SMILES: c1ccc(-c2ccc(N(c3ccccc3)c3ccc(N(c4ccccc4)c4ccc(-c5ccccc5)c(-c5ccccc5)c4)c4c3oc3cc5ccccc5cc34)cc2-c2ccccc2)cc1 ; c1ccc(-c2ccc(N(c3ccccc3)c3cc(N(c4ccccc4)c4ccc(-c5ccccc5)c(-c5ccccc5)c4)c4oc5cc6ccccc6cc5c4c3)cc2-c2ccccc2)cc1] 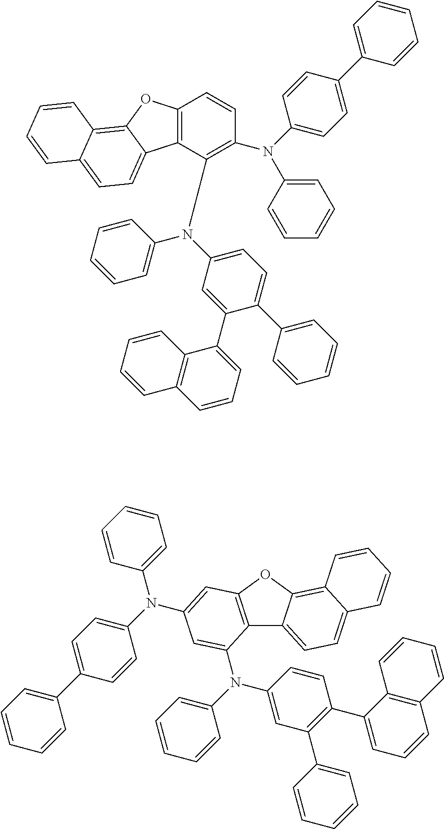 [SMILES: c1ccc(-c2ccc(N(c3ccccc3)c3ccc4oc5c6ccccc6ccc5c4c3N(c3ccccc3)c3ccc(-c4ccccc4)c(-c4cccc5ccccc45)c3)cc2)cc1 ; c1ccc(-c2ccc(N(c3ccccc3)c3cc(N(c4ccccc4)c4ccc(-c5cccc6ccccc56)c(-c5ccccc5)c4)c4c(c3)oc3c5ccccc5ccc34)cc2)cc1] 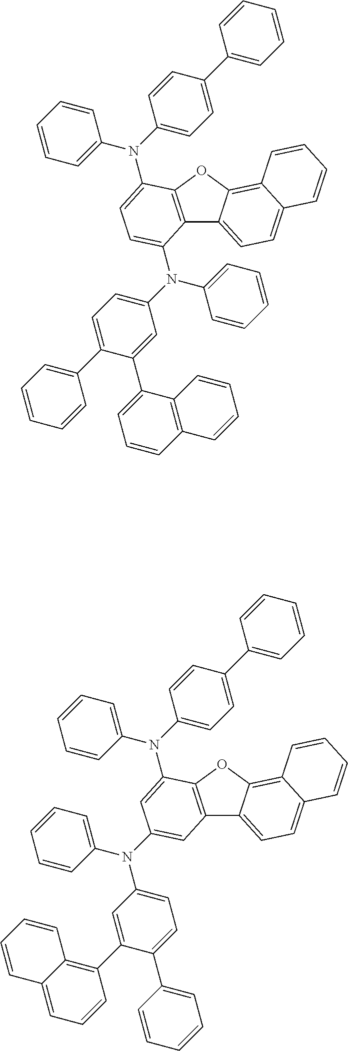 [SMILES: c1ccc(-c2ccc(N(c3ccccc3)c3ccc(N(c4ccccc4)c4ccc(-c5ccccc5)c(-c5cccc6ccccc56)c4)c4c3oc3c5ccccc5ccc34)cc2)cc1 ; c1ccc(-c2ccc(N(c3ccccc3)c3cc(N(c4ccccc4)c4ccc(-c5ccccc5)c(-c5cccc6ccccc56)c4)cc4c3oc3c5ccccc5ccc43)cc2)cc1] 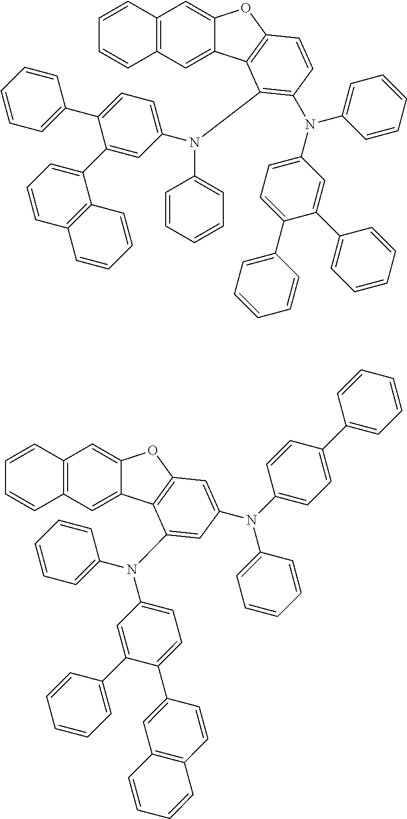 [SMILES: c1ccc(-c2ccc(N(c3ccccc3)c3ccc4oc5cc6ccccc6cc5c4c3N(c3ccccc3)c3ccc(-c4ccccc4)c(-c4cccc5ccccc45)c3)cc2-c2ccccc2)cc1 ; c1ccc(-c2ccc(N(c3ccccc3)c3cc(N(c4ccccc4)c4ccc(-c5ccc6ccccc6c5)c(-c5ccccc5)c4)c4c(c3)oc3cc5ccccc5cc34)cc2)cc1] 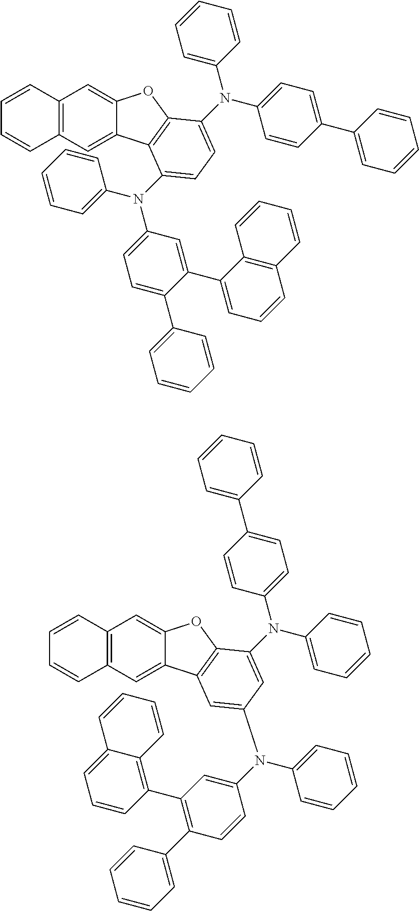 [SMILES: c1ccc(-c2ccc(N(c3ccccc3)c3ccc(N(c4ccccc4)c4ccc(-c5ccccc5)c(-c5cccc6ccccc56)c4)c4c3oc3cc5ccccc5cc34)cc2)cc1 ; c1ccc(-c2ccc(N(c3ccccc3)c3cc(N(c4ccccc4)c4ccc(-c5ccccc5)c(-c5cccc6ccccc56)c4)cc4c3oc3cc5ccccc5cc34)cc2)cc1] 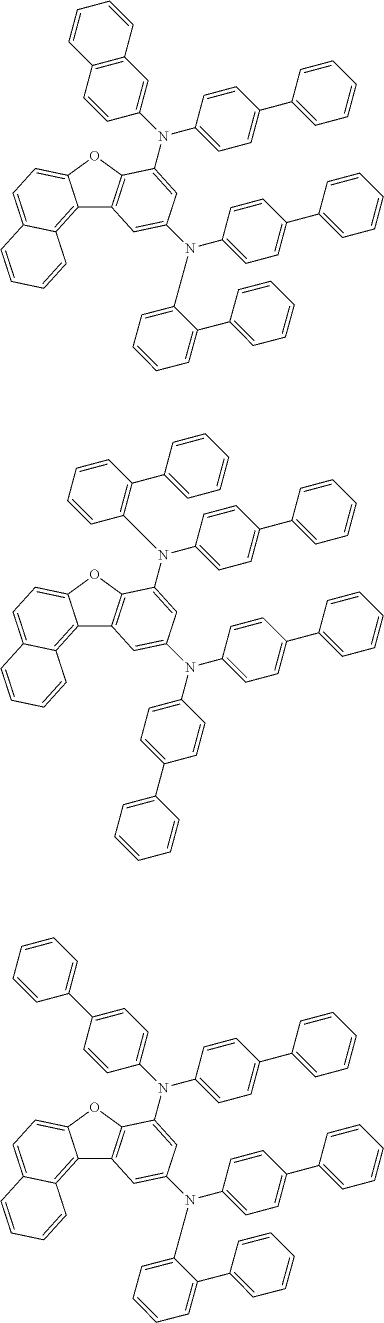 [SMILES: c1ccc(-c2ccc(N(c3cc(N(c4ccc(-c5ccccc5)cc4)c4ccc5ccccc5c4)c4oc5ccc6ccccc6c5c4c3)c3ccccc3-c3ccccc3)cc2)cc1 ; c1ccc(-c2ccc(N(c3ccc(-c4ccccc4)cc3)c3cc(N(c4ccc(-c5ccccc5)cc4)c4ccccc4-c4ccccc4)c4oc5ccc6ccccc6c5c4c3)cc2)cc1 ; c1ccc(-c2ccc(N(c3cc(N(c4ccc(-c5ccccc5)cc4)c4ccc(-c5ccccc5)cc4)c4oc5ccc6ccccc6c5c4c3)c3ccccc3-c3ccccc3)cc2)cc1] 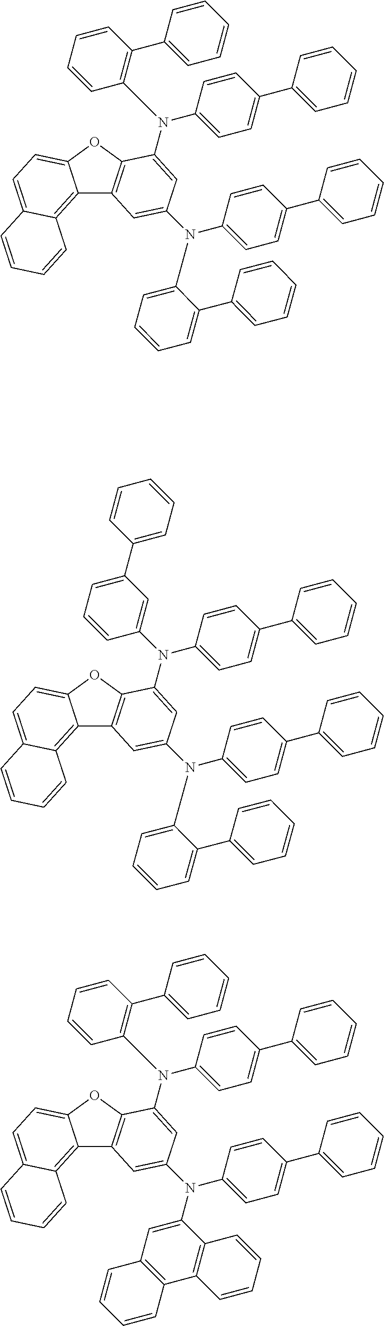 [SMILES: c1ccc(-c2ccc(N(c3cc(N(c4ccc(-c5ccccc5)cc4)c4ccccc4-c4ccccc4)c4oc5ccc6ccccc6c5c4c3)c3ccccc3-c3ccccc3)cc2)cc1 ; c1ccc(-c2ccc(N(c3cc(N(c4ccc(-c5ccccc5)cc4)c4cccc(-c5ccccc5)c4)c4oc5ccc6ccccc6c5c4c3)c3ccccc3-c3ccccc3)cc2)cc1 ; c1ccc(-c2ccc(N(c3cc(N(c4ccc(-c5ccccc5)cc4)c4ccccc4-c4ccccc4)c4oc5ccc6ccccc6c5c4c3)c3cc4ccccc4c4ccccc34)cc2)cc1] 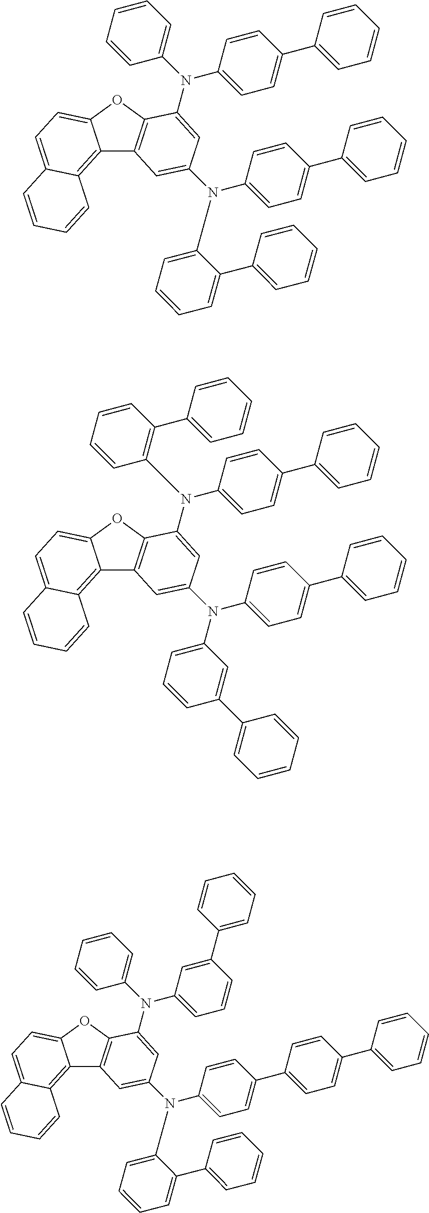 [SMILES: c1ccc(-c2ccc(N(c3cc(N(c4ccccc4)c4ccc(-c5ccccc5)cc4)c4oc5ccc6ccccc6c5c4c3)c3ccccc3-c3ccccc3)cc2)cc1 ; c1ccc(-c2ccc(N(c3cccc(-c4ccccc4)c3)c3cc(N(c4ccc(-c5ccccc5)cc4)c4ccccc4-c4ccccc4)c4oc5ccc6ccccc6c5c4c3)cc2)cc1 ; c1ccc(-c2ccc(-c3ccc(N(c4cc(N(c5ccccc5)c5cccc(-c6ccccc6)c5)c5oc6ccc7ccccc7c6c5c4)c4ccccc4-c4ccccc4)cc3)cc2)cc1] 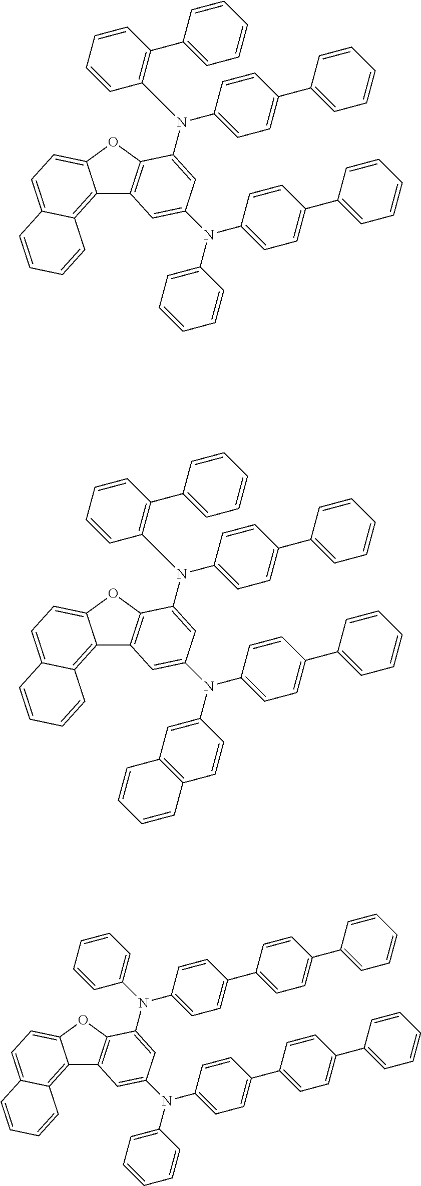 [SMILES: c1ccc(-c2ccc(N(c3ccccc3)c3cc(N(c4ccc(-c5ccccc5)cc4)c4ccccc4-c4ccccc4)c4oc5ccc6ccccc6c5c4c3)cc2)cc1 ; c1ccc(-c2ccc(N(c3ccc4ccccc4c3)c3cc(N(c4ccc(-c5ccccc5)cc4)c4ccccc4-c4ccccc4)c4oc5ccc6ccccc6c5c4c3)cc2)cc1 ; c1ccc(-c2ccc(-c3ccc(N(c4ccccc4)c4cc(N(c5ccccc5)c5ccc(-c6ccc(-c7ccccc7)cc6)cc5)c5oc6ccc7ccccc7c6c5c4)cc3)cc2)cc1] 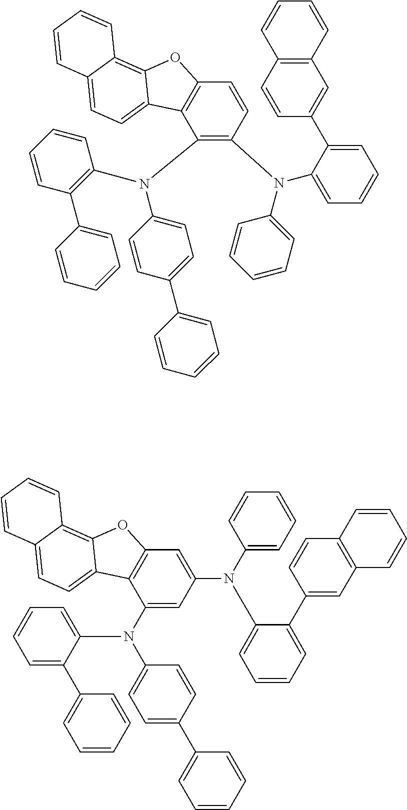 [SMILES: c1ccc(-c2ccc(N(c3ccccc3-c3ccccc3)c3c(N(c4ccccc4)c4ccccc4-c4ccc5ccccc5c4)ccc4oc5c6ccccc6ccc5c34)cc2)cc1 ; c1ccc(-c2ccc(N(c3ccccc3-c3ccccc3)c3cc(N(c4ccccc4)c4ccccc4-c4ccc5ccccc5c4)cc4oc5c6ccccc6ccc5c34)cc2)cc1] 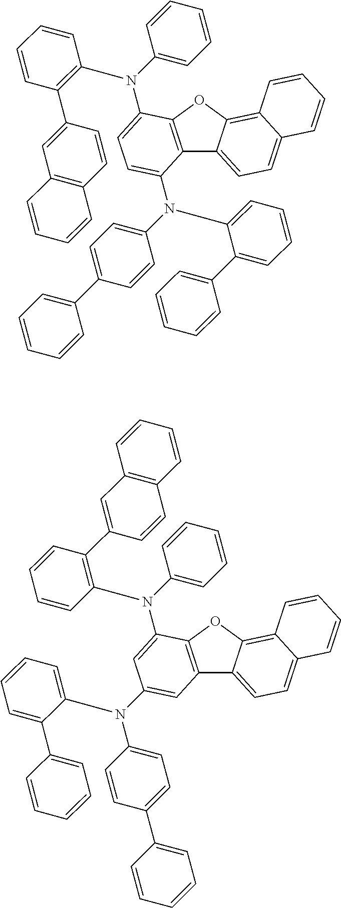 [SMILES: c1ccc(-c2ccc(N(c3ccccc3-c3ccccc3)c3ccc(N(c4ccccc4)c4ccccc4-c4ccc5ccccc5c4)c4oc5c6ccccc6ccc5c34)cc2)cc1 ; c1ccc(-c2ccc(N(c3cc(N(c4ccccc4)c4ccccc4-c4ccc5ccccc5c4)c4oc5c6ccccc6ccc5c4c3)c3ccccc3-c3ccccc3)cc2)cc1] 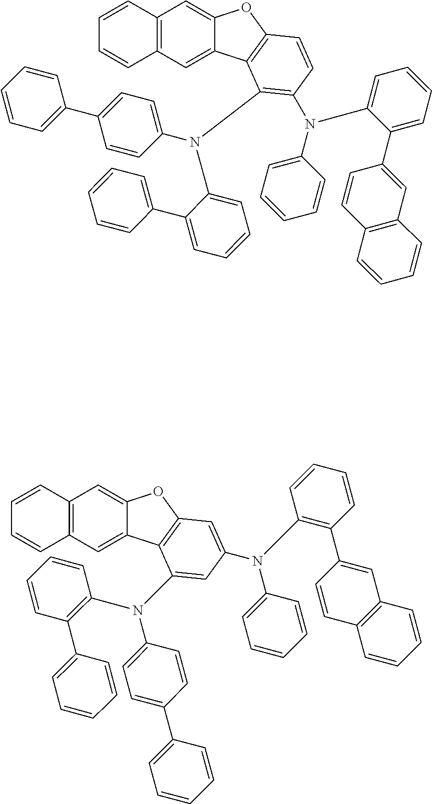 [SMILES: c1ccc(-c2ccc(N(c3ccccc3-c3ccccc3)c3c(N(c4ccccc4)c4ccccc4-c4ccc5ccccc5c4)ccc4oc5cc6ccccc6cc5c34)cc2)cc1 ; c1ccc(-c2ccc(N(c3ccccc3-c3ccccc3)c3cc(N(c4ccccc4)c4ccccc4-c4ccc5ccccc5c4)cc4oc5cc6ccccc6cc5c34)cc2)cc1] 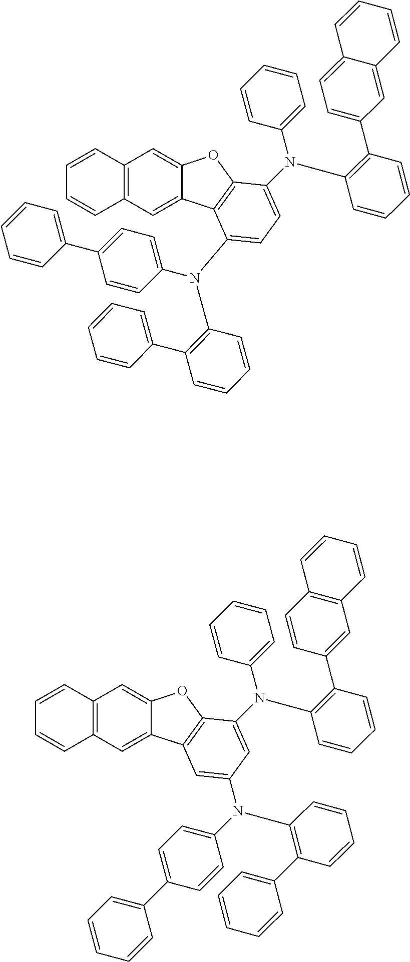 [SMILES: c1ccc(-c2ccc(N(c3ccccc3-c3ccccc3)c3ccc(N(c4ccccc4)c4ccccc4-c4ccc5ccccc5c4)c4oc5cc6ccccc6cc5c34)cc2)cc1 ; c1ccc(-c2ccc(N(c3cc(N(c4ccccc4)c4ccccc4-c4ccc5ccccc5c4)c4oc5cc6ccccc6cc5c4c3)c3ccccc3-c3ccccc3)cc2)cc1] 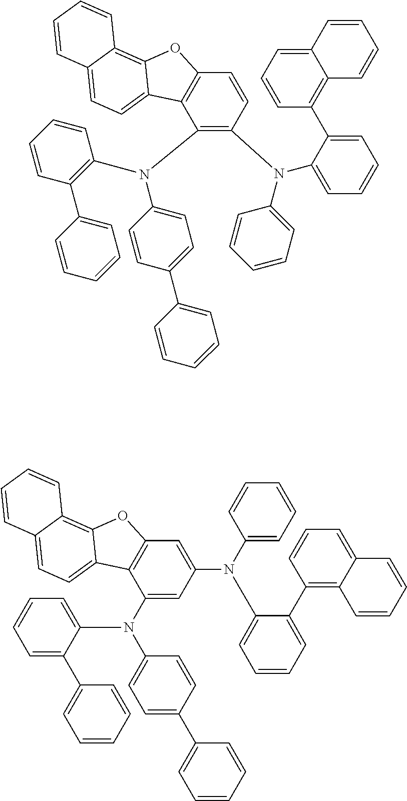 [SMILES: c1ccc(-c2ccc(N(c3ccccc3-c3ccccc3)c3c(N(c4ccccc4)c4ccccc4-c4cccc5ccccc45)ccc4oc5c6ccccc6ccc5c34)cc2)cc1 ; c1ccc(-c2ccc(N(c3ccccc3-c3ccccc3)c3cc(N(c4ccccc4)c4ccccc4-c4cccc5ccccc45)cc4oc5c6ccccc6ccc5c34)cc2)cc1] 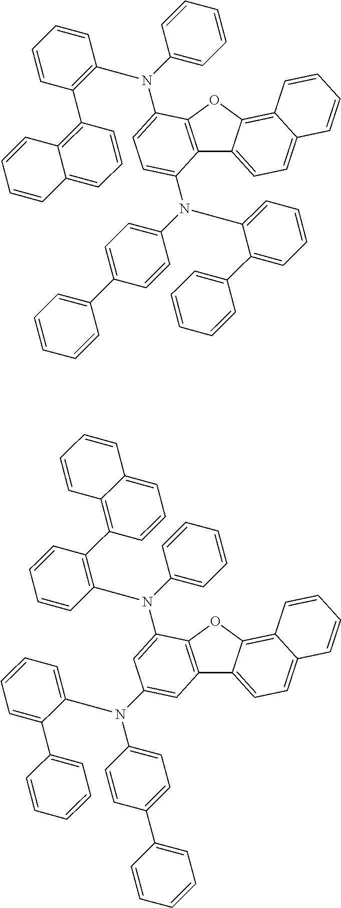 [SMILES: c1ccc(-c2ccc(N(c3ccccc3-c3ccccc3)c3ccc(N(c4ccccc4)c4ccccc4-c4cccc5ccccc45)c4oc5c6ccccc6ccc5c34)cc2)cc1 ; c1ccc(-c2ccc(N(c3cc(N(c4ccccc4)c4ccccc4-c4cccc5ccccc45)c4oc5c6ccccc6ccc5c4c3)c3ccccc3-c3ccccc3)cc2)cc1] 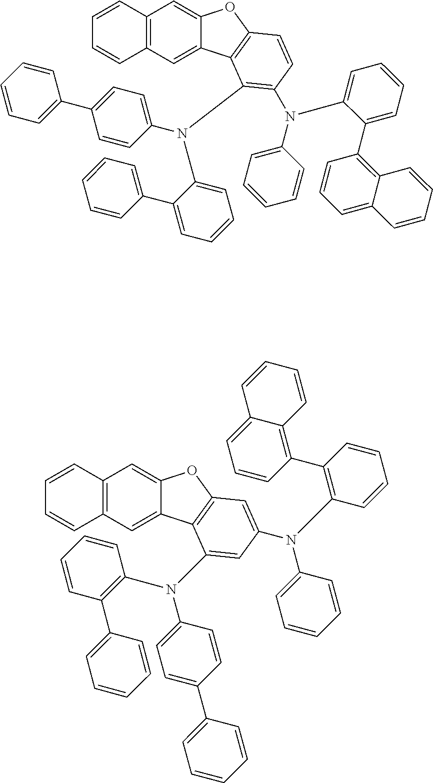 [SMILES: c1ccc(-c2ccc(N(c3ccccc3-c3ccccc3)c3c(N(c4ccccc4)c4ccccc4-c4cccc5ccccc45)ccc4oc5cc6ccccc6cc5c34)cc2)cc1 ; c1ccc(-c2ccc(N(c3ccccc3-c3ccccc3)c3cc(N(c4ccccc4)c4ccccc4-c4cccc5ccccc45)cc4oc5cc6ccccc6cc5c34)cc2)cc1] 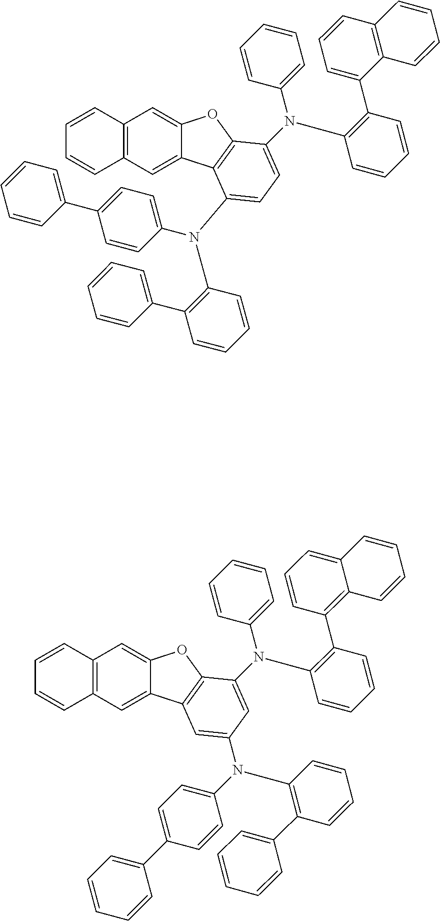 [SMILES: c1ccc(-c2ccc(N(c3ccccc3-c3ccccc3)c3ccc(N(c4ccccc4)c4ccccc4-c4cccc5ccccc45)c4oc5cc6ccccc6cc5c34)cc2)cc1 ; c1ccc(-c2ccc(N(c3cc(N(c4ccccc4)c4ccccc4-c4cccc5ccccc45)c4oc5cc6ccccc6cc5c4c3)c3ccccc3-c3ccccc3)cc2)cc1] 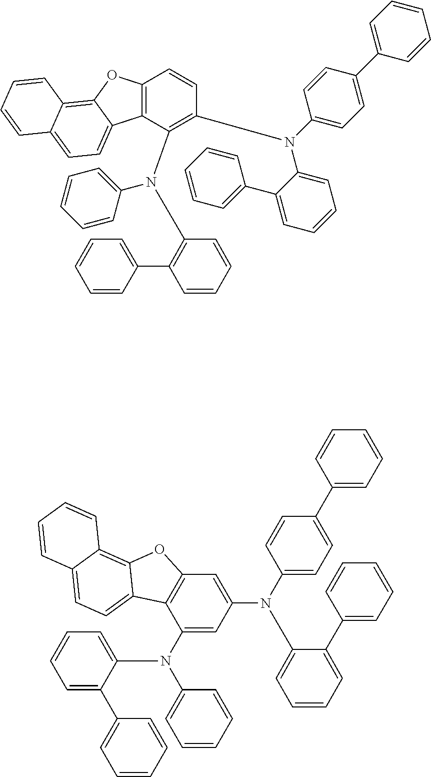 [SMILES: c1ccc(-c2ccc(N(c3ccccc3-c3ccccc3)c3ccc4oc5c6ccccc6ccc5c4c3N(c3ccccc3)c3ccccc3-c3ccccc3)cc2)cc1 ; c1ccc(-c2ccc(N(c3cc(N(c4ccccc4)c4ccccc4-c4ccccc4)c4c(c3)oc3c5ccccc5ccc34)c3ccccc3-c3ccccc3)cc2)cc1] 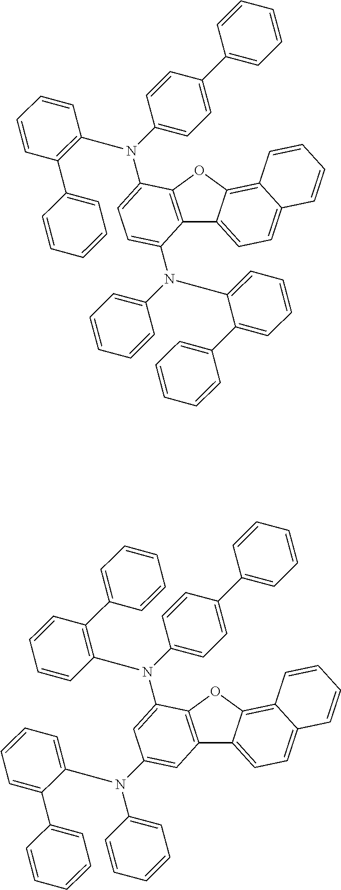 [SMILES: c1ccc(-c2ccc(N(c3ccccc3-c3ccccc3)c3ccc(N(c4ccccc4)c4ccccc4-c4ccccc4)c4c3oc3c5ccccc5ccc34)cc2)cc1 ; c1ccc(-c2ccc(N(c3ccccc3-c3ccccc3)c3cc(N(c4ccccc4)c4ccccc4-c4ccccc4)cc4c3oc3c5ccccc5ccc43)cc2)cc1] 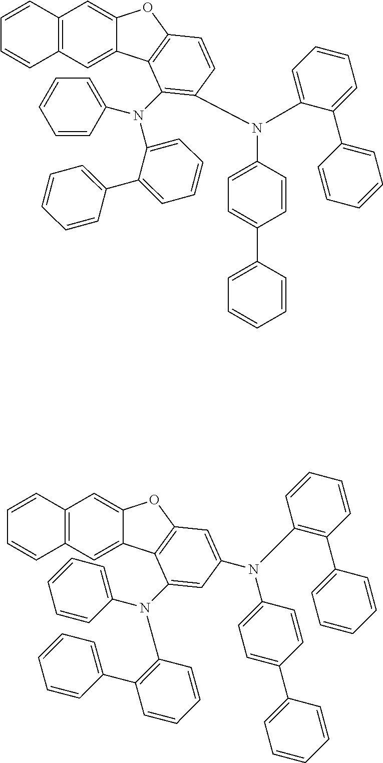 [SMILES: c1ccc(-c2ccc(N(c3ccccc3-c3ccccc3)c3ccc4oc5cc6ccccc6cc5c4c3N(c3ccccc3)c3ccccc3-c3ccccc3)cc2)cc1 ; c1ccc(-c2ccc(N(c3cc(N(c4ccccc4)c4ccccc4-c4ccccc4)c4c(c3)oc3cc5ccccc5cc34)c3ccccc3-c3ccccc3)cc2)cc1] 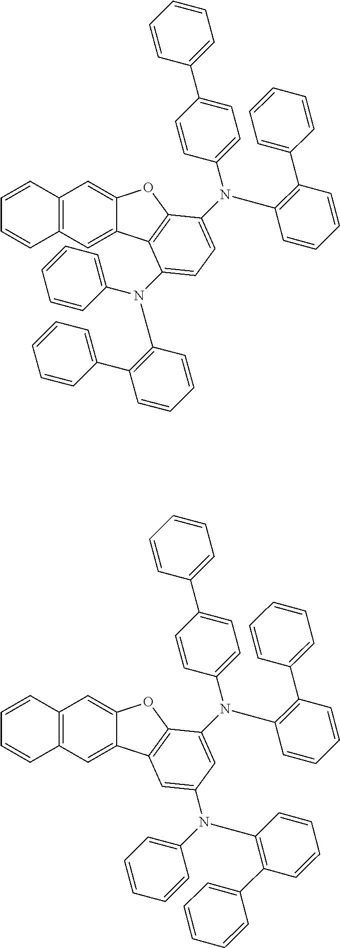 [SMILES: c1ccc(-c2ccc(N(c3ccccc3-c3ccccc3)c3ccc(N(c4ccccc4)c4ccccc4-c4ccccc4)c4c3oc3cc5ccccc5cc34)cc2)cc1 ; c1ccc(-c2ccc(N(c3ccccc3-c3ccccc3)c3cc(N(c4ccccc4)c4ccccc4-c4ccccc4)cc4c3oc3cc5ccccc5cc34)cc2)cc1] 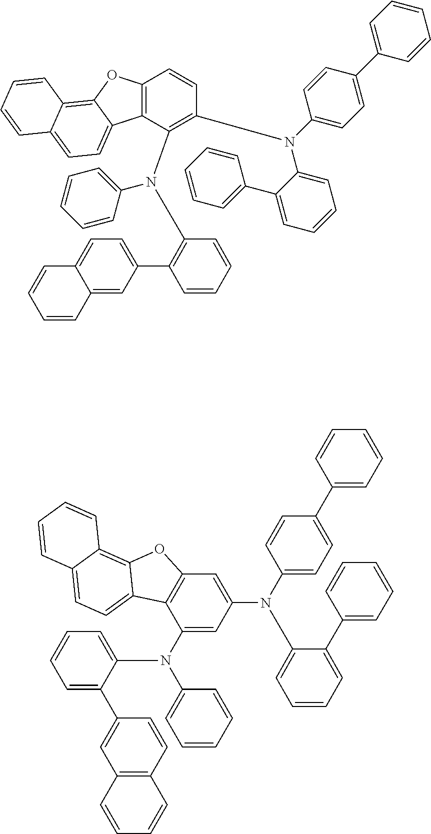 [SMILES: c1ccc(-c2ccc(N(c3ccccc3-c3ccccc3)c3ccc4oc5c6ccccc6ccc5c4c3N(c3ccccc3)c3ccccc3-c3ccc4ccccc4c3)cc2)cc1 ; c1ccc(-c2ccc(N(c3cc(N(c4ccccc4)c4ccccc4-c4ccc5ccccc5c4)c4c(c3)oc3c5ccccc5ccc34)c3ccccc3-c3ccccc3)cc2)cc1] 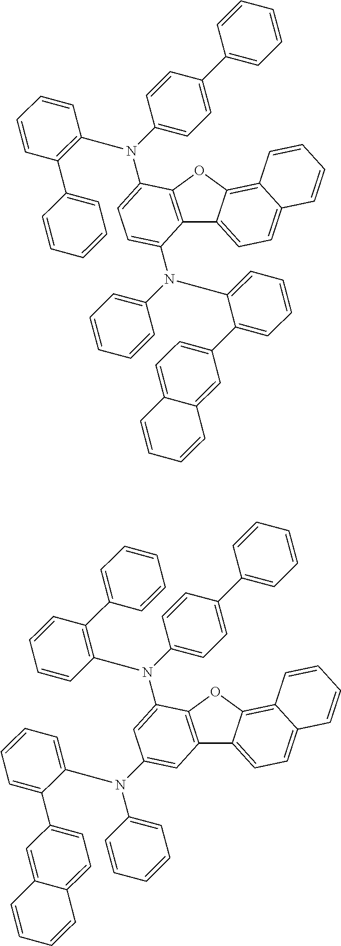 [SMILES: c1ccc(-c2ccc(N(c3ccccc3-c3ccccc3)c3ccc(N(c4ccccc4)c4ccccc4-c4ccc5ccccc5c4)c4c3oc3c5ccccc5ccc34)cc2)cc1 ; c1ccc(-c2ccc(N(c3ccccc3-c3ccccc3)c3cc(N(c4ccccc4)c4ccccc4-c4ccc5ccccc5c4)cc4c3oc3c5ccccc5ccc43)cc2)cc1] 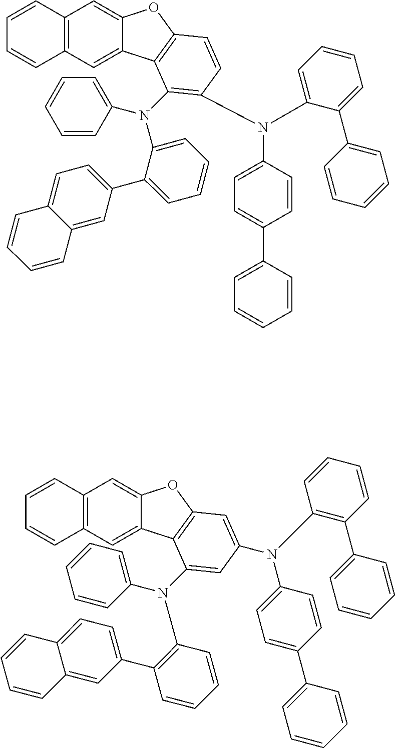 [SMILES: c1ccc(-c2ccc(N(c3ccccc3-c3ccccc3)c3ccc4oc5cc6ccccc6cc5c4c3N(c3ccccc3)c3ccccc3-c3ccc4ccccc4c3)cc2)cc1 ; c1ccc(-c2ccc(N(c3cc(N(c4ccccc4)c4ccccc4-c4ccc5ccccc5c4)c4c(c3)oc3cc5ccccc5cc34)c3ccccc3-c3ccccc3)cc2)cc1] 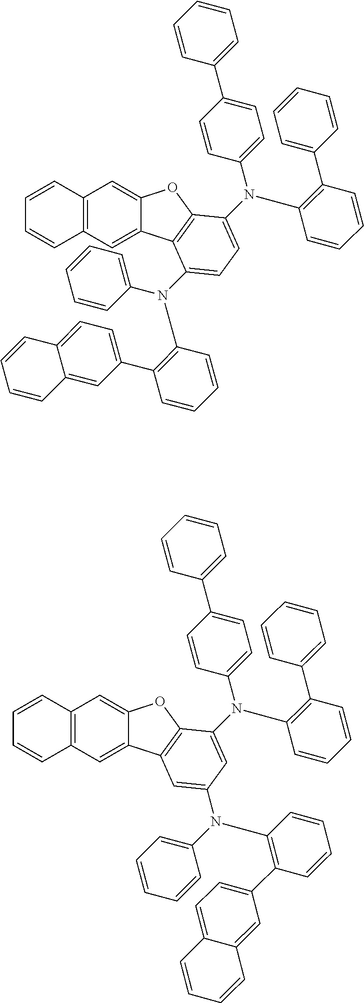 [SMILES: c1ccc(-c2ccc(N(c3ccccc3-c3ccccc3)c3ccc(N(c4ccccc4)c4ccccc4-c4ccc5ccccc5c4)c4c3oc3cc5ccccc5cc34)cc2)cc1 ; c1ccc(-c2ccc(N(c3ccccc3-c3ccccc3)c3cc(N(c4ccccc4)c4ccccc4-c4ccc5ccccc5c4)cc4c3oc3cc5ccccc5cc34)cc2)cc1] 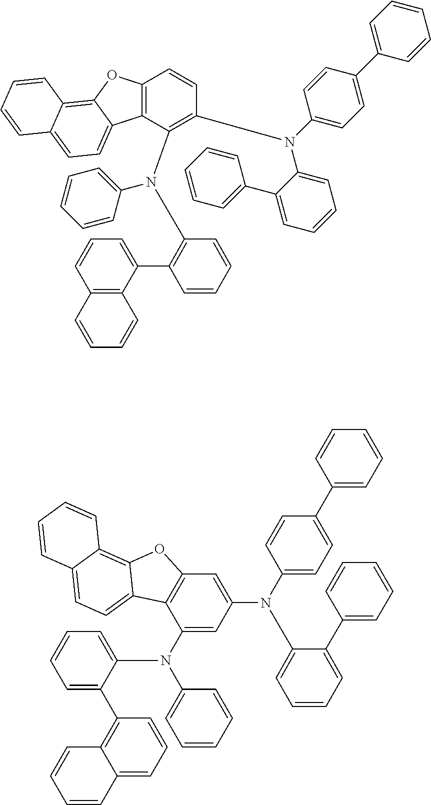 [SMILES: c1ccc(-c2ccc(N(c3ccccc3-c3ccccc3)c3ccc4oc5c6ccccc6ccc5c4c3N(c3ccccc3)c3ccccc3-c3cccc4ccccc34)cc2)cc1 ; c1ccc(-c2ccc(N(c3cc(N(c4ccccc4)c4ccccc4-c4cccc5ccccc45)c4c(c3)oc3c5ccccc5ccc34)c3ccccc3-c3ccccc3)cc2)cc1] 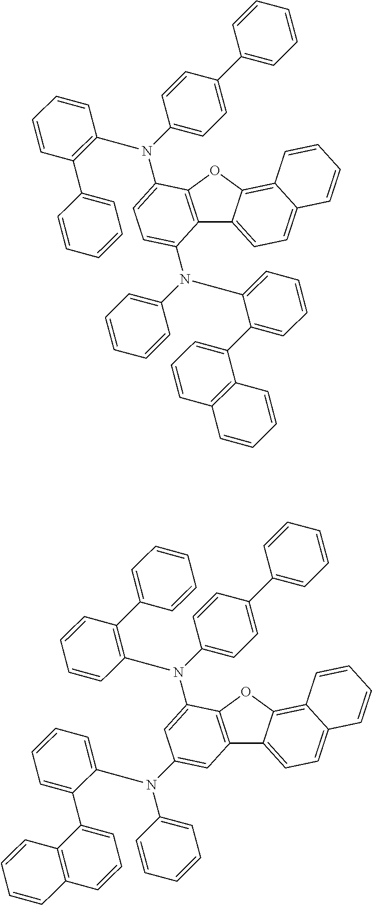 [SMILES: c1ccc(-c2ccc(N(c3ccccc3-c3ccccc3)c3ccc(N(c4ccccc4)c4ccccc4-c4cccc5ccccc45)c4c3oc3c5ccccc5ccc34)cc2)cc1 ; c1ccc(-c2ccc(N(c3ccccc3-c3ccccc3)c3cc(N(c4ccccc4)c4ccccc4-c4cccc5ccccc45)cc4c3oc3c5ccccc5ccc43)cc2)cc1] 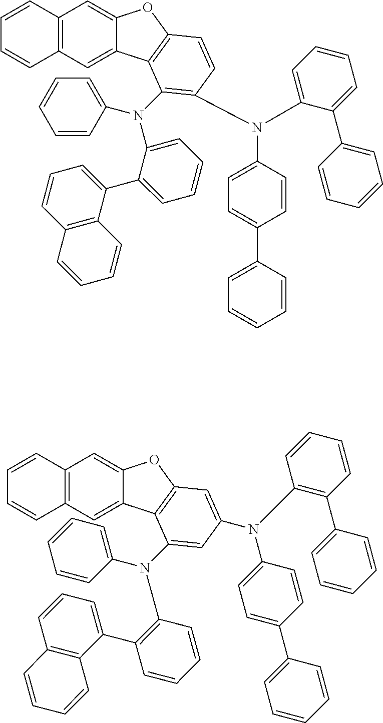 [SMILES: c1ccc(-c2ccc(N(c3ccccc3-c3ccccc3)c3ccc4oc5cc6ccccc6cc5c4c3N(c3ccccc3)c3ccccc3-c3cccc4ccccc34)cc2)cc1 ; c1ccc(-c2ccc(N(c3cc(N(c4ccccc4)c4ccccc4-c4cccc5ccccc45)c4c(c3)oc3cc5ccccc5cc34)c3ccccc3-c3ccccc3)cc2)cc1] 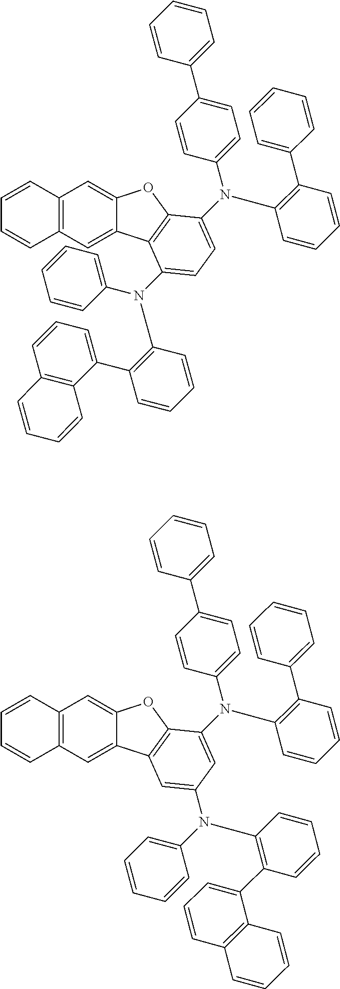 [SMILES: c1ccc(-c2ccc(N(c3ccccc3-c3ccccc3)c3ccc(N(c4ccccc4)c4ccccc4-c4cccc5ccccc45)c4c3oc3cc5ccccc5cc34)cc2)cc1 ; c1ccc(-c2ccc(N(c3ccccc3-c3ccccc3)c3cc(N(c4ccccc4)c4ccccc4-c4cccc5ccccc45)cc4c3oc3cc5ccccc5cc34)cc2)cc1] 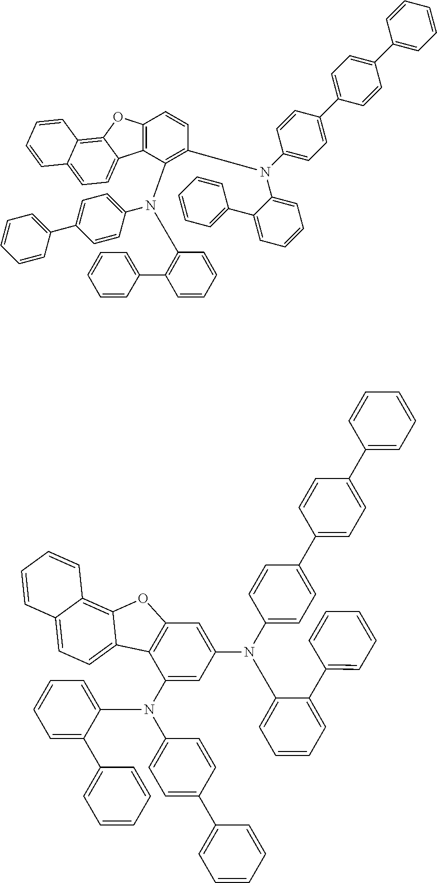 [SMILES: c1ccc(-c2ccc(-c3ccc(N(c4ccccc4-c4ccccc4)c4ccc5oc6c7ccccc7ccc6c5c4N(c4ccc(-c5ccccc5)cc4)c4ccccc4-c4ccccc4)cc3)cc2)cc1 ; c1ccc(-c2ccc(-c3ccc(N(c4cc(N(c5ccc(-c6ccccc6)cc5)c5ccccc5-c5ccccc5)c5c(c4)oc4c6ccccc6ccc45)c4ccccc4-c4ccccc4)cc3)cc2)cc1] 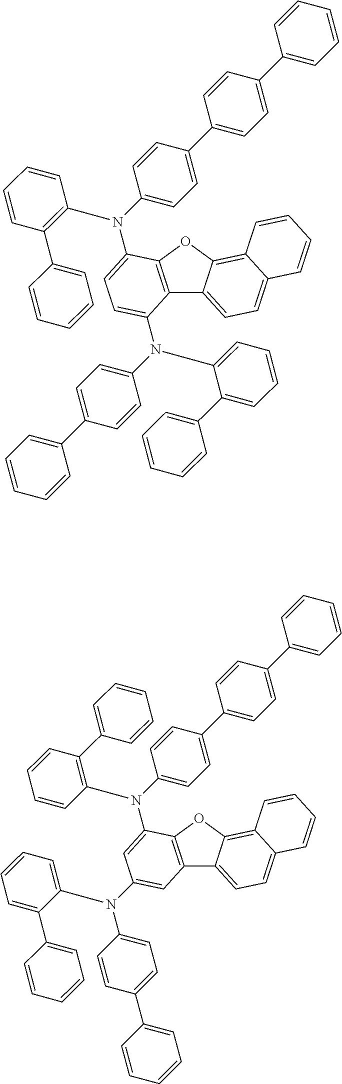 [SMILES: c1ccc(-c2ccc(-c3ccc(N(c4ccccc4-c4ccccc4)c4ccc(N(c5ccc(-c6ccccc6)cc5)c5ccccc5-c5ccccc5)c5c4oc4c6ccccc6ccc45)cc3)cc2)cc1 ; c1ccc(-c2ccc(-c3ccc(N(c4ccccc4-c4ccccc4)c4cc(N(c5ccc(-c6ccccc6)cc5)c5ccccc5-c5ccccc5)cc5c4oc4c6ccccc6ccc54)cc3)cc2)cc1] 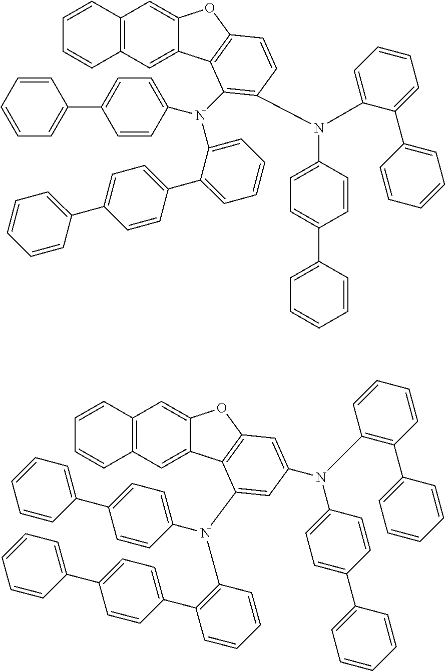 [SMILES: c1ccc(-c2ccc(-c3ccccc3N(c3ccc(-c4ccccc4)cc3)c3c(N(c4ccc(-c5ccccc5)cc4)c4ccccc4-c4ccccc4)ccc4oc5cc6ccccc6cc5c34)cc2)cc1 ; c1ccc(-c2ccc(-c3ccccc3N(c3ccc(-c4ccccc4)cc3)c3cc(N(c4ccc(-c5ccccc5)cc4)c4ccccc4-c4ccccc4)cc4oc5cc6ccccc6cc5c34)cc2)cc1] 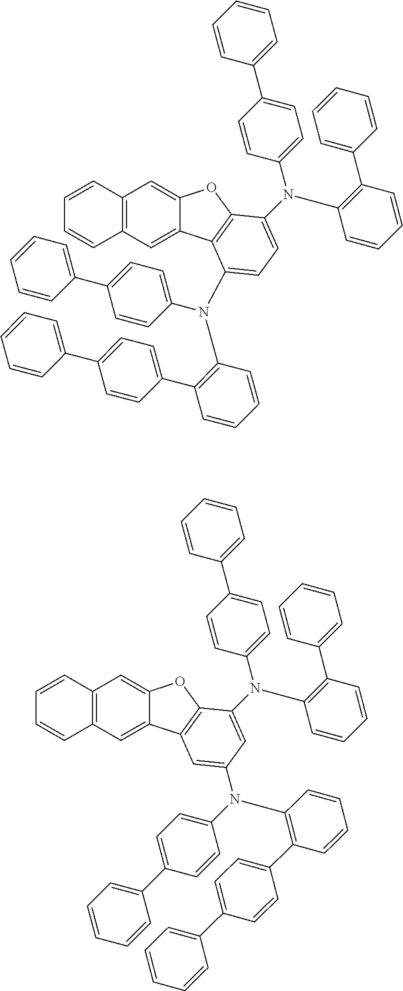 [SMILES: c1ccc(-c2ccc(-c3ccccc3N(c3ccc(-c4ccccc4)cc3)c3ccc(N(c4ccc(-c5ccccc5)cc4)c4ccccc4-c4ccccc4)c4oc5cc6ccccc6cc5c34)cc2)cc1 ; c1ccc(-c2ccc(-c3ccccc3N(c3ccc(-c4ccccc4)cc3)c3cc(N(c4ccc(-c5ccccc5)cc4)c4ccccc4-c4ccccc4)c4oc5cc6ccccc6cc5c4c3)cc2)cc1] 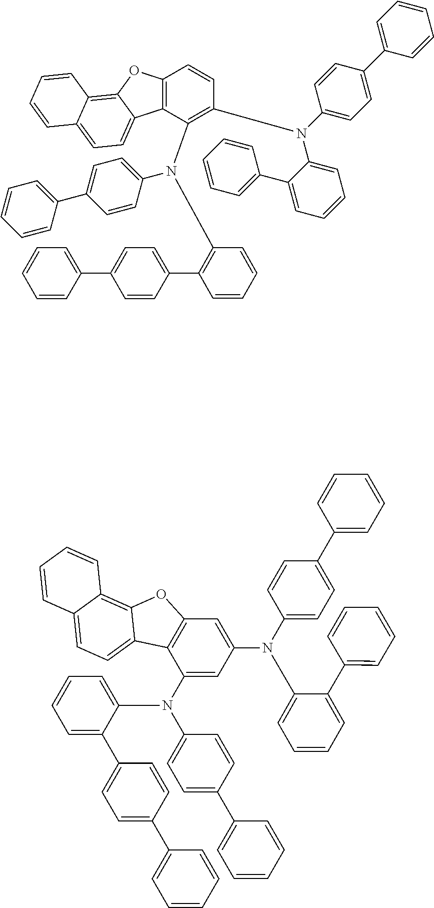 [SMILES: c1ccc(-c2ccc(-c3ccccc3N(c3ccc(-c4ccccc4)cc3)c3c(N(c4ccc(-c5ccccc5)cc4)c4ccccc4-c4ccccc4)ccc4oc5c6ccccc6ccc5c34)cc2)cc1 ; c1ccc(-c2ccc(-c3ccccc3N(c3ccc(-c4ccccc4)cc3)c3cc(N(c4ccc(-c5ccccc5)cc4)c4ccccc4-c4ccccc4)cc4oc5c6ccccc6ccc5c34)cc2)cc1] 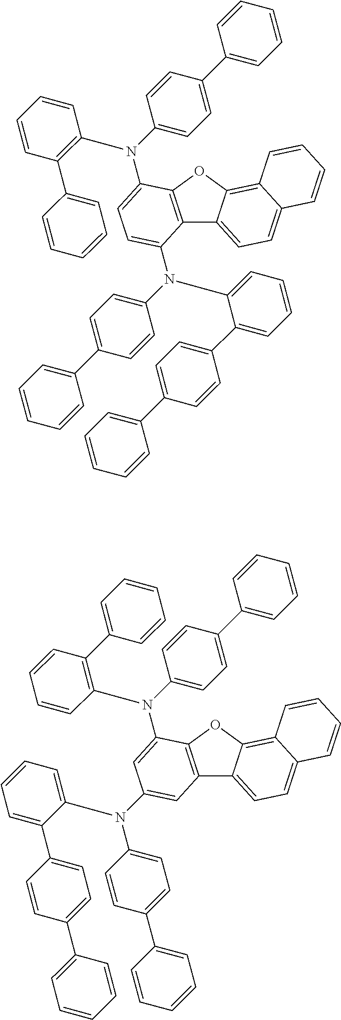 [SMILES: c1ccc(-c2ccc(-c3ccccc3N(c3ccc(-c4ccccc4)cc3)c3ccc(N(c4ccc(-c5ccccc5)cc4)c4ccccc4-c4ccccc4)c4oc5c6ccccc6ccc5c34)cc2)cc1 ; c1ccc(-c2ccc(-c3ccccc3N(c3ccc(-c4ccccc4)cc3)c3cc(N(c4ccc(-c5ccccc5)cc4)c4ccccc4-c4ccccc4)c4oc5c6ccccc6ccc5c4c3)cc2)cc1] 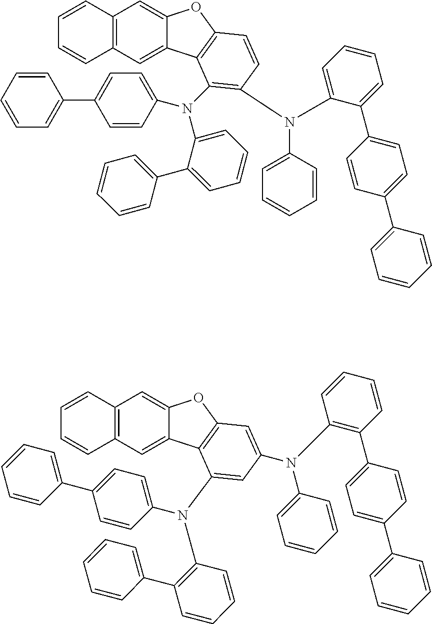 [SMILES: c1ccc(-c2ccc(-c3ccccc3N(c3ccccc3)c3ccc4oc5cc6ccccc6cc5c4c3N(c3ccc(-c4ccccc4)cc3)c3ccccc3-c3ccccc3)cc2)cc1 ; c1ccc(-c2ccc(-c3ccccc3N(c3ccccc3)c3cc(N(c4ccc(-c5ccccc5)cc4)c4ccccc4-c4ccccc4)c4c(c3)oc3cc5ccccc5cc34)cc2)cc1] 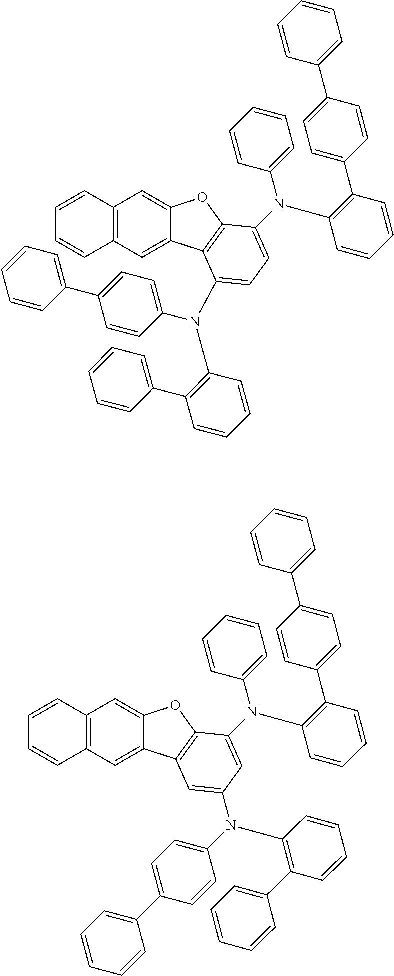 [SMILES: c1ccc(-c2ccc(-c3ccccc3N(c3ccccc3)c3ccc(N(c4ccc(-c5ccccc5)cc4)c4ccccc4-c4ccccc4)c4c3oc3cc5ccccc5cc34)cc2)cc1 ; c1ccc(-c2ccc(-c3ccccc3N(c3ccccc3)c3cc(N(c4ccc(-c5ccccc5)cc4)c4ccccc4-c4ccccc4)cc4c3oc3cc5ccccc5cc34)cc2)cc1] 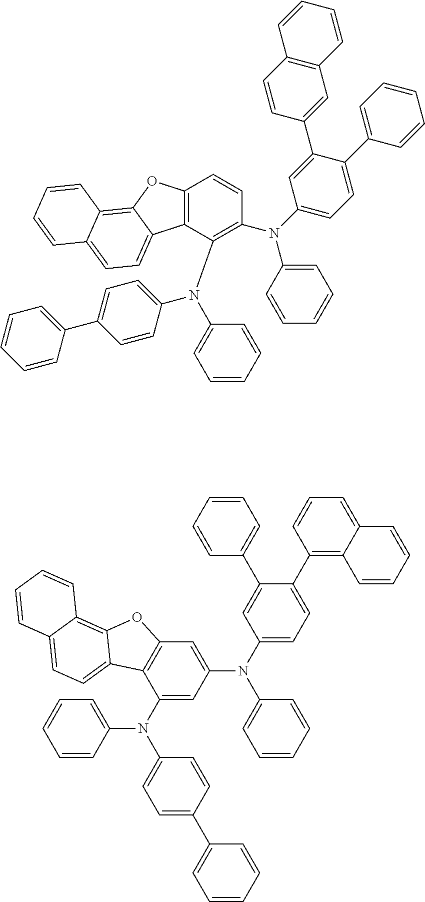 [SMILES: c1ccc(-c2ccc(N(c3ccccc3)c3c(N(c4ccccc4)c4ccc(-c5ccccc5)c(-c5ccc6ccccc6c5)c4)ccc4oc5c6ccccc6ccc5c34)cc2)cc1 ; c1ccc(-c2ccc(N(c3ccccc3)c3cc(N(c4ccccc4)c4ccc(-c5cccc6ccccc56)c(-c5ccccc5)c4)cc4oc5c6ccccc6ccc5c34)cc2)cc1] 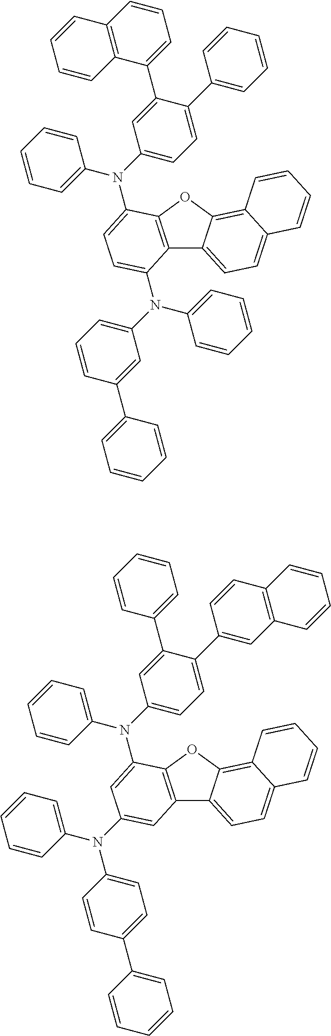 [SMILES: c1ccc(-c2cccc(N(c3ccccc3)c3ccc(N(c4ccccc4)c4ccc(-c5ccccc5)c(-c5cccc6ccccc56)c4)c4oc5c6ccccc6ccc5c34)c2)cc1 ; c1ccc(-c2ccc(N(c3ccccc3)c3cc(N(c4ccccc4)c4ccc(-c5ccc6ccccc6c5)c(-c5ccccc5)c4)c4oc5c6ccccc6ccc5c4c3)cc2)cc1] 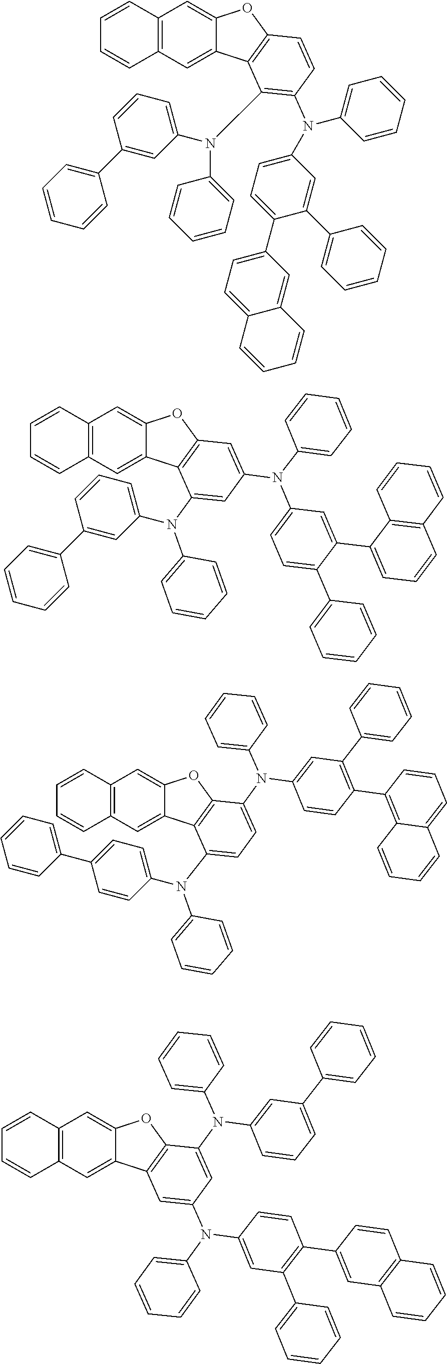 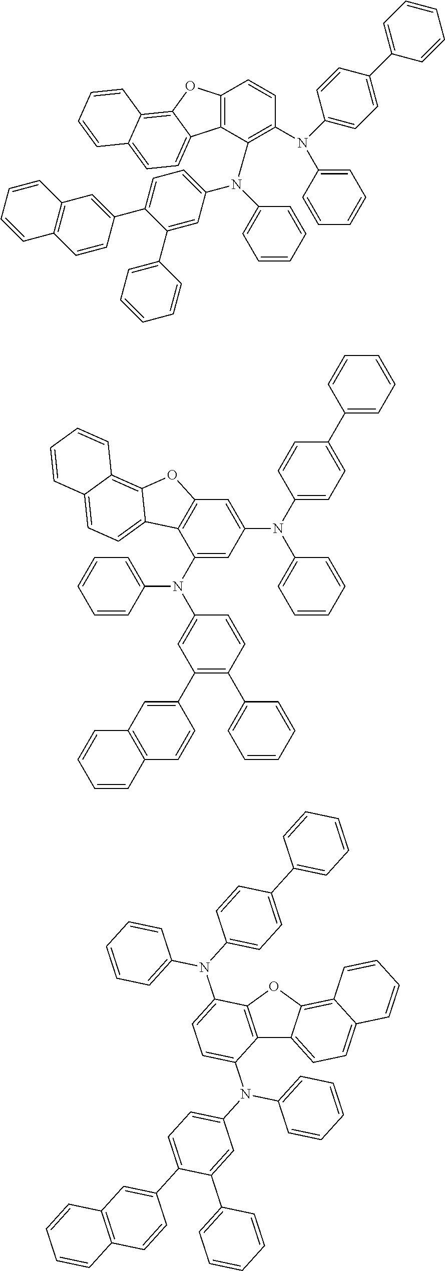 [SMILES: c1ccc(-c2ccc(N(c3ccccc3)c3ccc4oc5c6ccccc6ccc5c4c3N(c3ccccc3)c3ccc(-c4ccc5ccccc5c4)c(-c4ccccc4)c3)cc2)cc1 ; c1ccc(-c2ccc(N(c3ccccc3)c3cc(N(c4ccccc4)c4ccc(-c5ccccc5)c(-c5ccc6ccccc6c5)c4)c4c(c3)oc3c5ccccc5ccc34)cc2)cc1 ; c1ccc(-c2ccc(N(c3ccccc3)c3ccc(N(c4ccccc4)c4ccc(-c5ccc6ccccc6c5)c(-c5ccccc5)c4)c4c3oc3c5ccccc5ccc34)cc2)cc1] 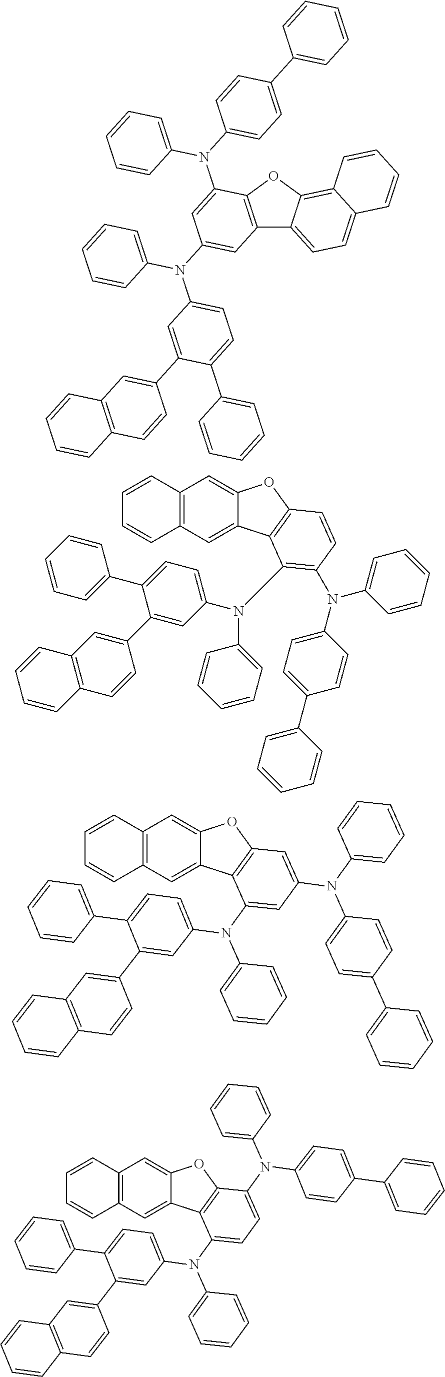 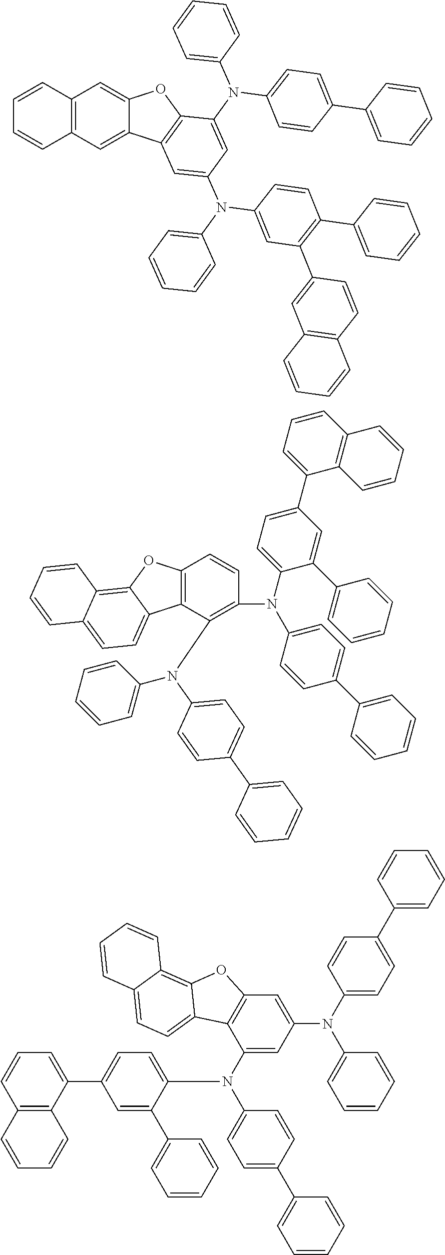 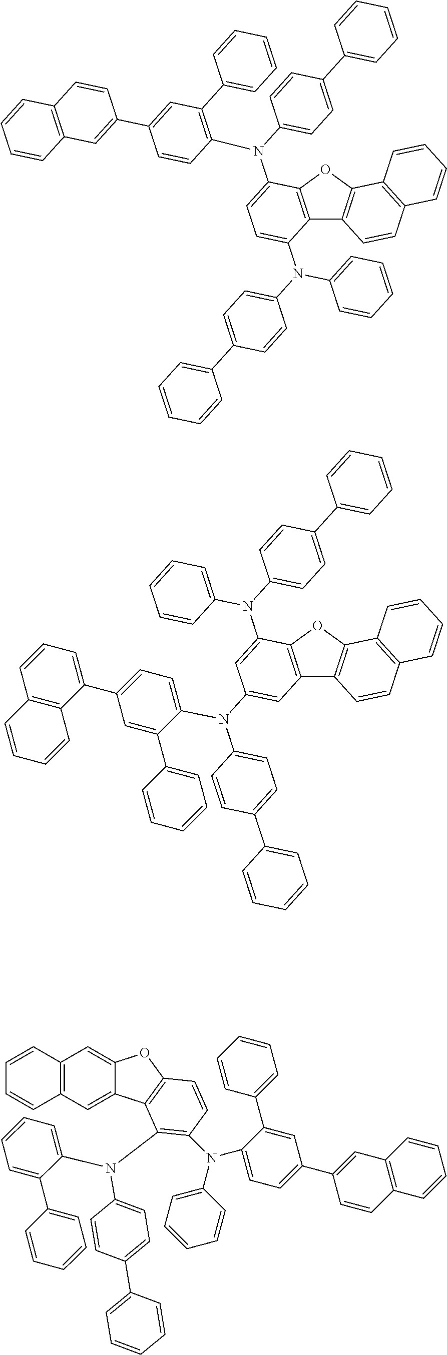 [SMILES: c1ccc(-c2ccc(N(c3ccc(-c4ccc5ccccc5c4)cc3-c3ccccc3)c3ccc(N(c4ccccc4)c4ccc(-c5ccccc5)cc4)c4c3oc3c5ccccc5ccc34)cc2)cc1 ; c1ccc(-c2ccc(N(c3cc(N(c4ccccc4)c4ccc(-c5ccccc5)cc4)c4oc5c6ccccc6ccc5c4c3)c3ccc(-c4cccc5ccccc45)cc3-c3ccccc3)cc2)cc1 ; c1ccc(-c2ccc(N(c3ccccc3-c3ccccc3)c3c(N(c4ccccc4)c4ccc(-c5ccc6ccccc6c5)cc4-c4ccccc4)ccc4oc5cc6ccccc6cc5c34)cc2)cc1] 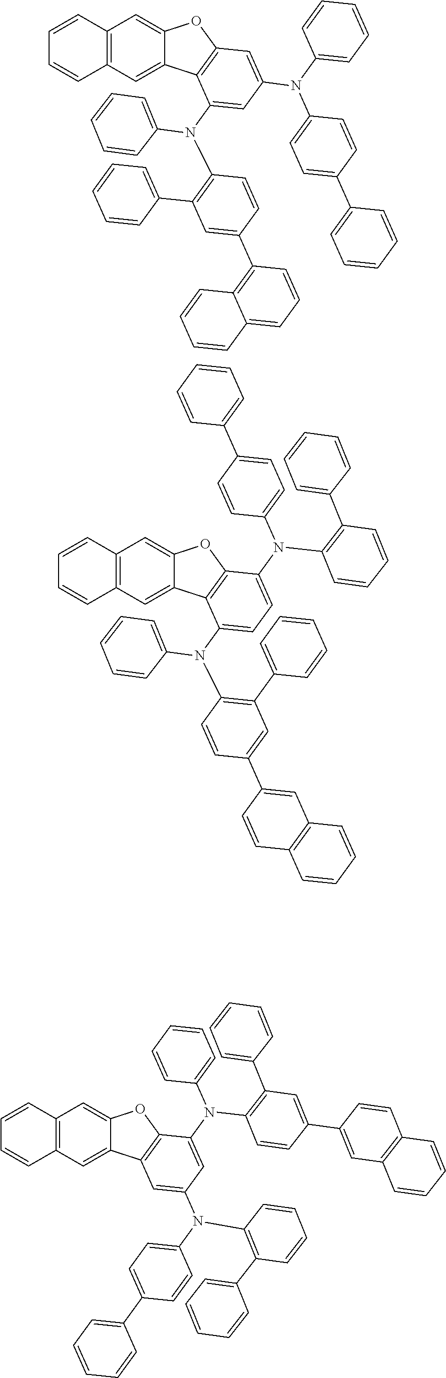 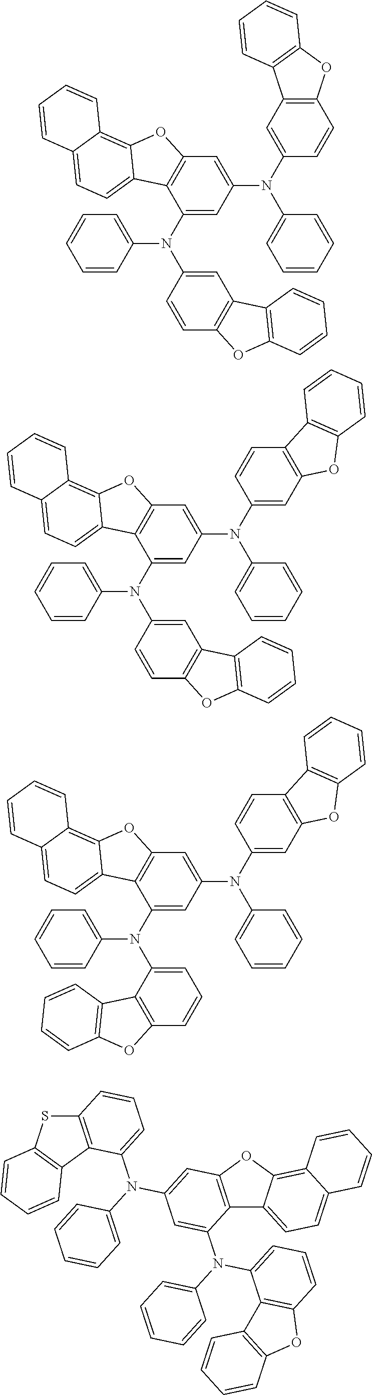 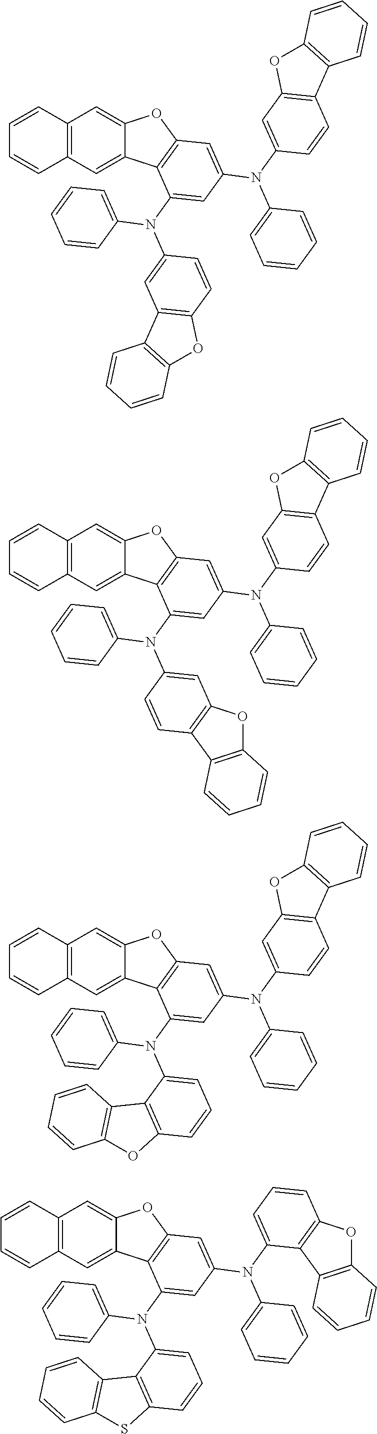 [SMILES: c1ccc(N(c2ccc3c(c2)oc2ccccc23)c2cc(N(c3ccccc3)c3ccc4oc5ccccc5c4c3)c3c(c2)oc2cc4ccccc4cc23)cc1 ; c1ccc(N(c2ccc3c(c2)oc2ccccc23)c2cc(N(c3ccccc3)c3ccc4c(c3)oc3ccccc34)c3c(c2)oc2cc4ccccc4cc23)cc1 ; c1ccc(N(c2ccc3c(c2)oc2ccccc23)c2cc(N(c3ccccc3)c3cccc4oc5ccccc5c34)c3c(c2)oc2cc4ccccc4cc23)cc1 ; c1ccc(N(c2cc(N(c3ccccc3)c3cccc4sc5ccccc5c34)c3c(c2)oc2cc4ccccc4cc23)c2cccc3oc4ccccc4c23)cc1] 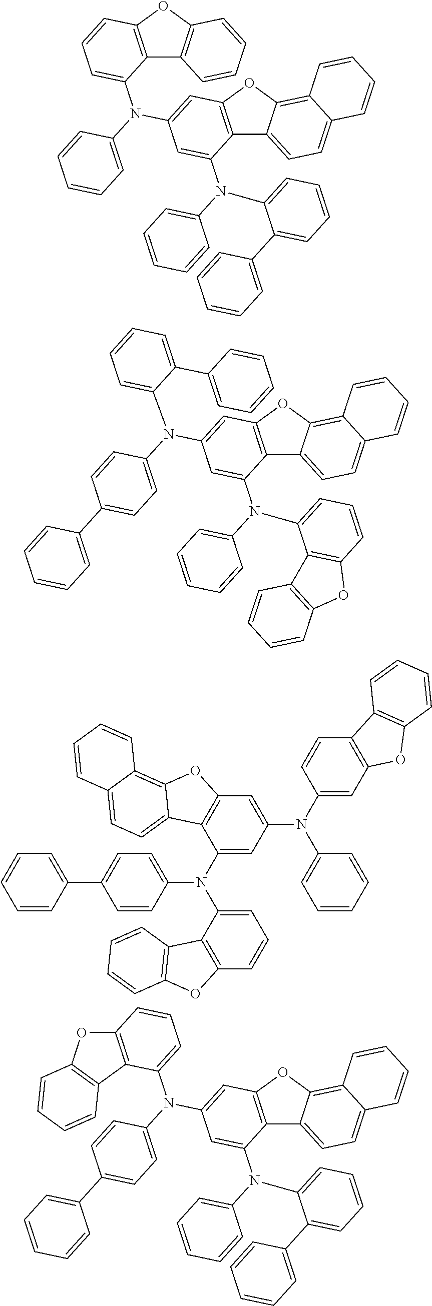 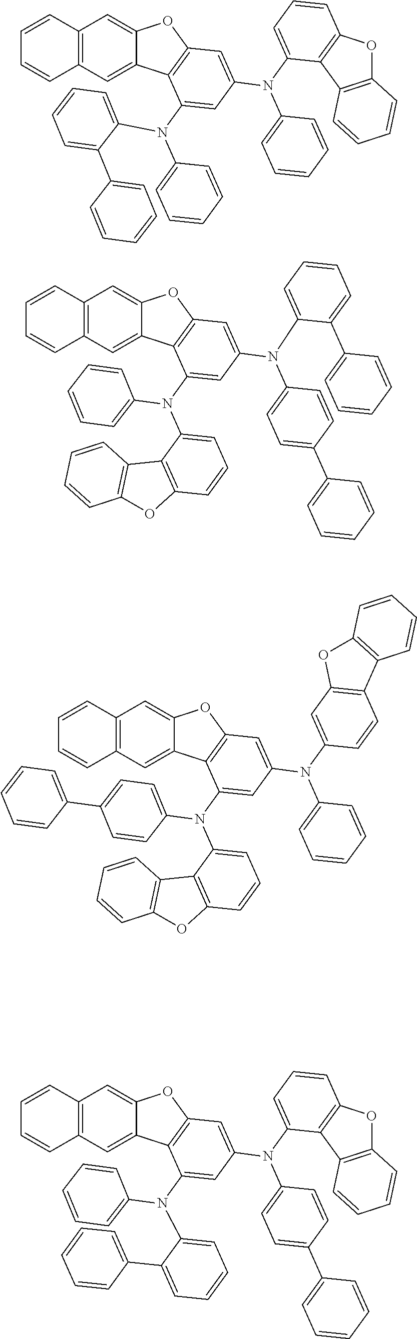 [SMILES: c1ccc(-c2ccccc2N(c2ccccc2)c2cc(N(c3ccccc3)c3cccc4oc5ccccc5c34)cc3oc4cc5ccccc5cc4c23)cc1 ; c1ccc(-c2ccc(N(c3cc(N(c4ccccc4)c4cccc5oc6ccccc6c45)c4c(c3)oc3cc5ccccc5cc34)c3ccccc3-c3ccccc3)cc2)cc1 ; c1ccc(-c2ccc(N(c3cccc4oc5ccccc5c34)c3cc(N(c4ccccc4)c4ccc5c(c4)oc4ccccc45)cc4oc5cc6ccccc6cc5c34)cc2)cc1 ; c1ccc(-c2ccc(N(c3cc(N(c4ccccc4)c4ccccc4-c4ccccc4)c4c(c3)oc3cc5ccccc5cc34)c3cccc4oc5ccccc5c34)cc2)cc1] 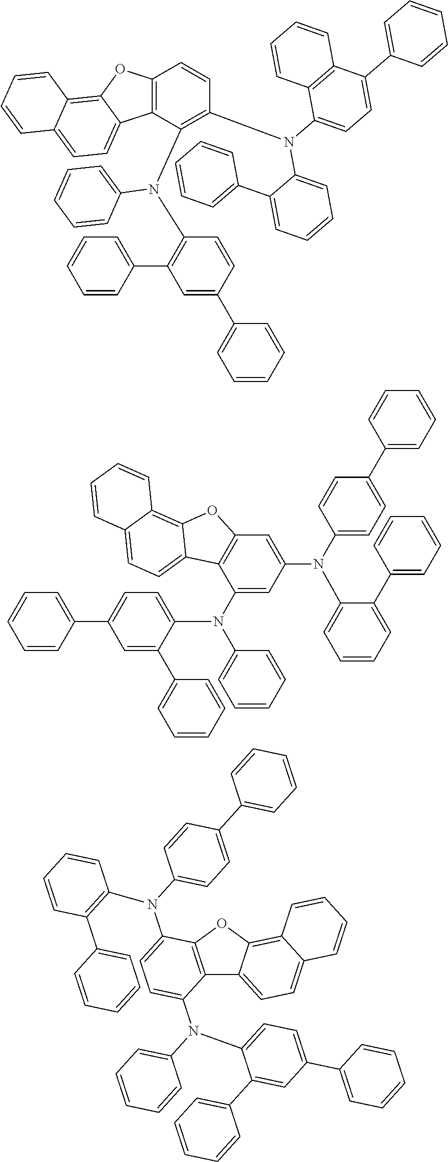 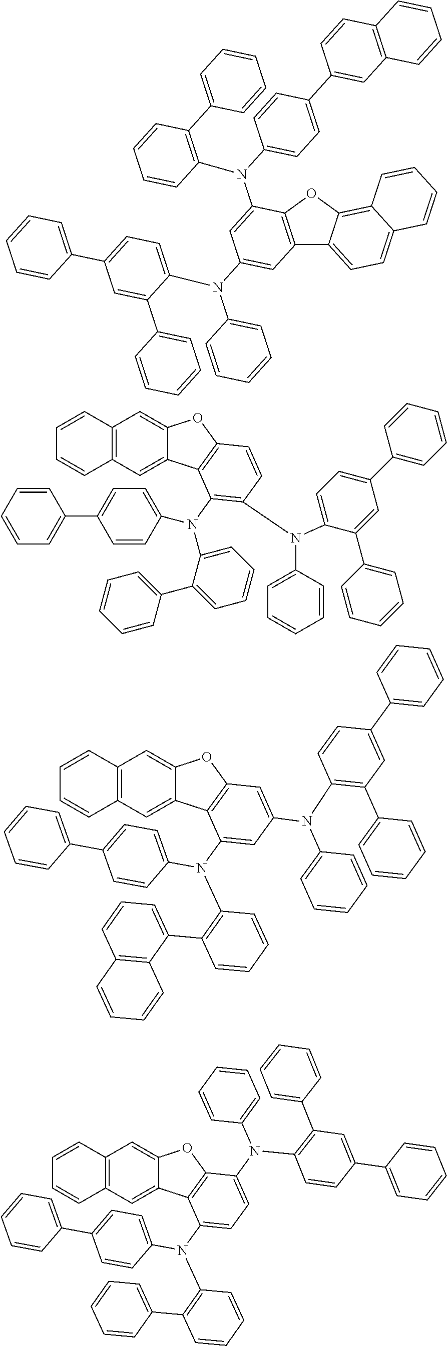 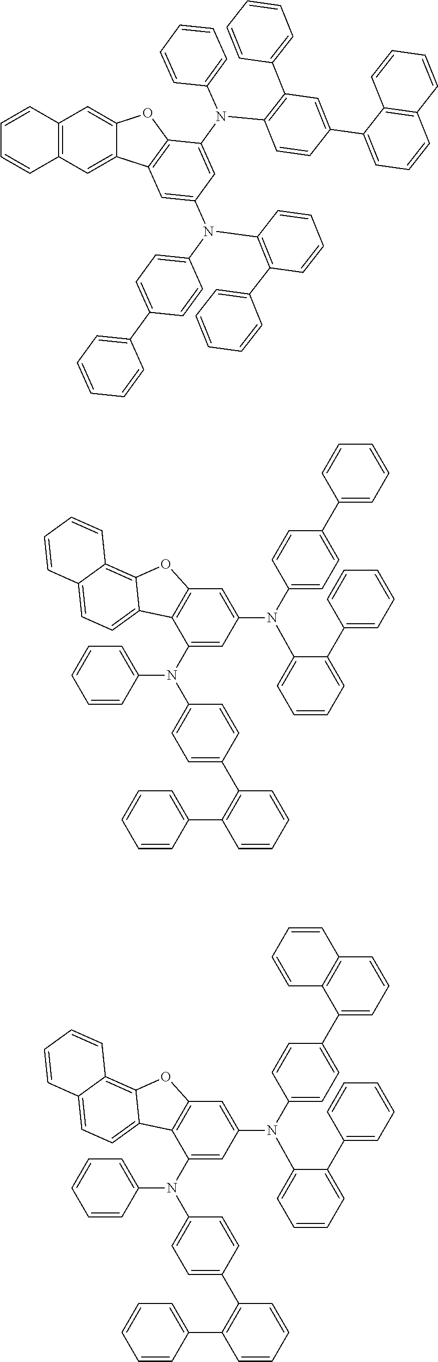 [SMILES: c1ccc(-c2ccc(N(c3cc(N(c4ccccc4)c4ccc(-c5cccc6ccccc56)cc4-c4ccccc4)c4oc5cc6ccccc6cc5c4c3)c3ccccc3-c3ccccc3)cc2)cc1 ; c1ccc(-c2ccc(N(c3cc(N(c4ccccc4)c4ccc(-c5ccccc5-c5ccccc5)cc4)c4c(c3)oc3c5ccccc5ccc34)c3ccccc3-c3ccccc3)cc2)cc1 ; c1ccc(-c2ccccc2-c2ccc(N(c3ccccc3)c3cc(N(c4ccc(-c5cccc6ccccc56)cc4)c4ccccc4-c4ccccc4)cc4oc5c6ccccc6ccc5c34)cc2)cc1] 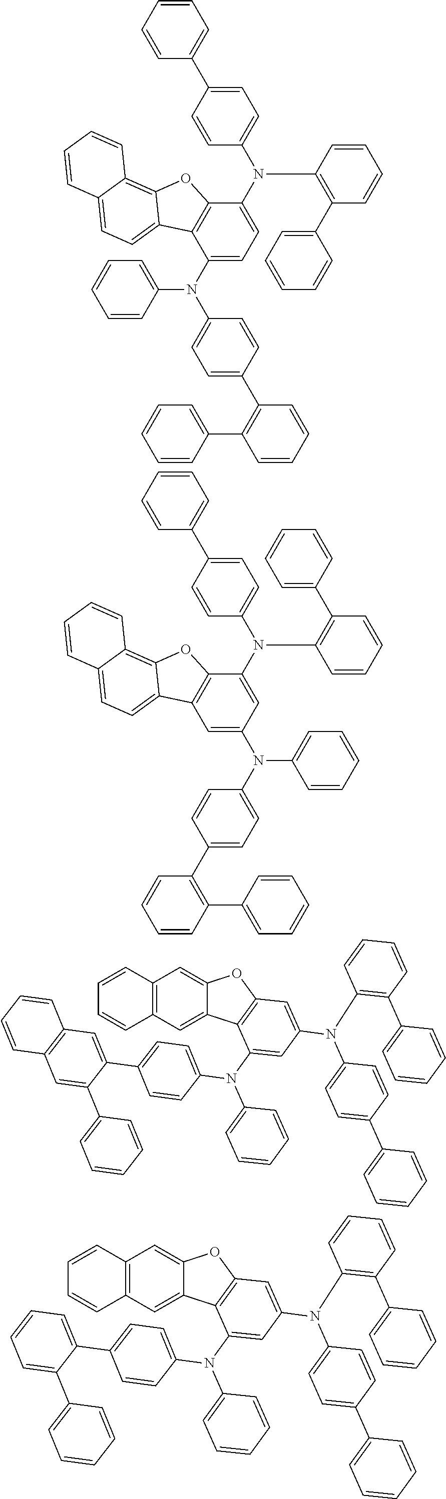 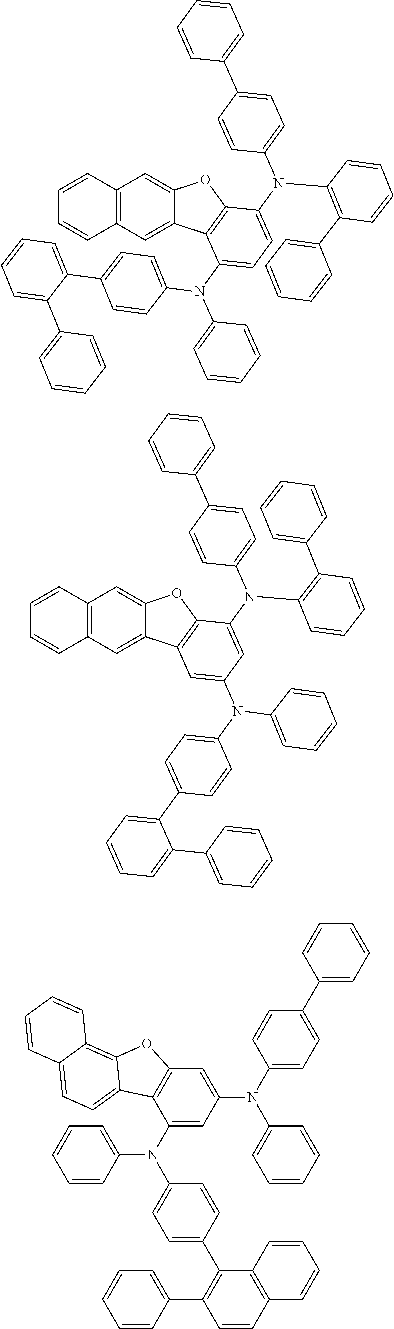 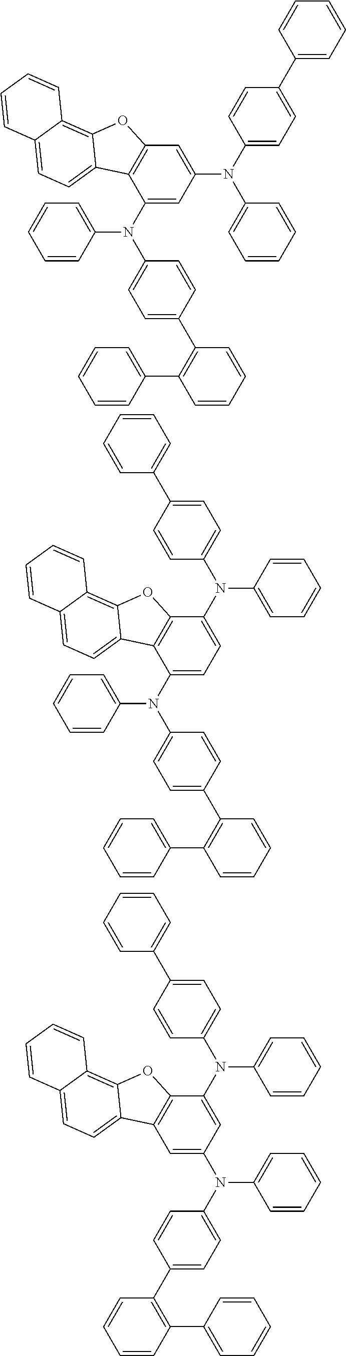 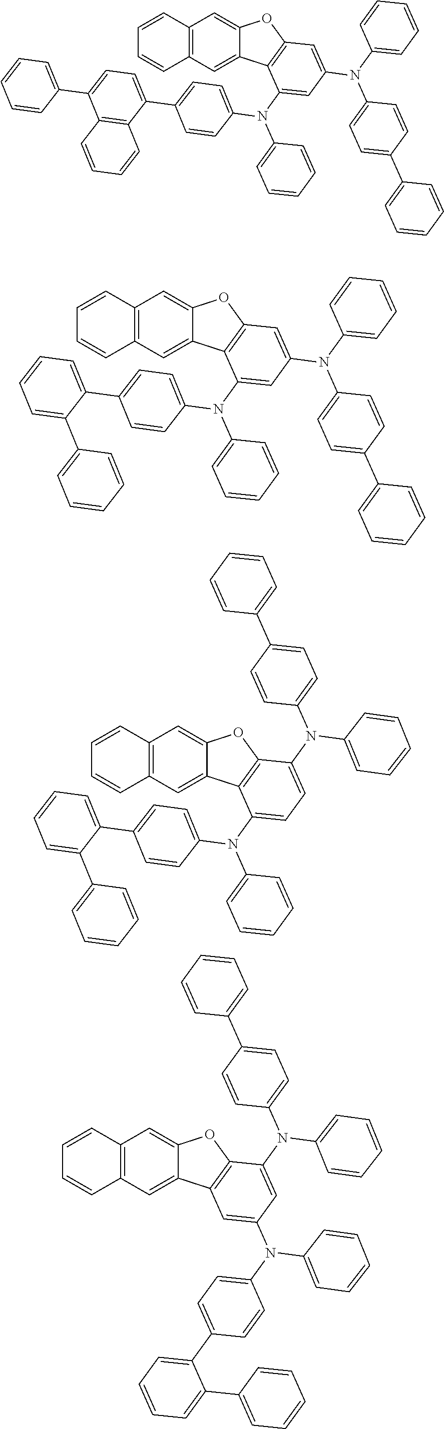 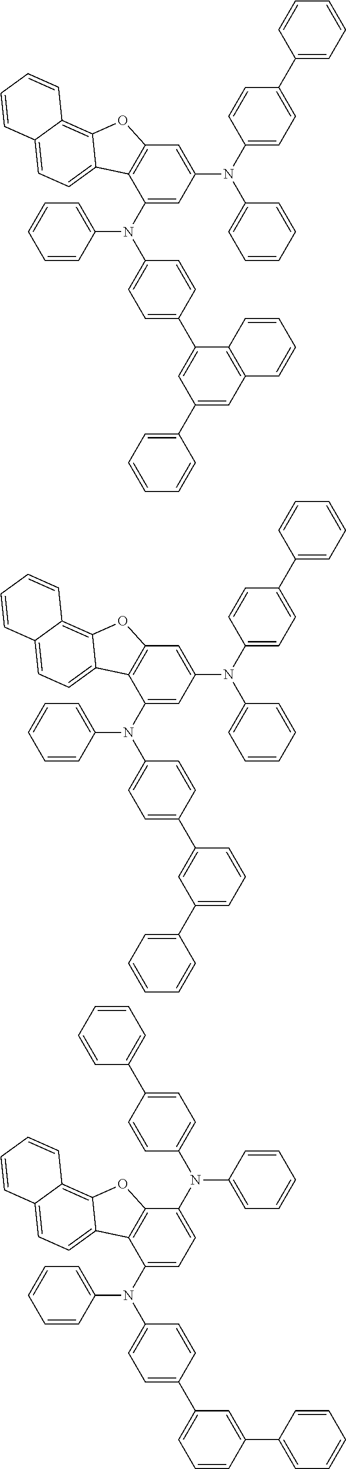 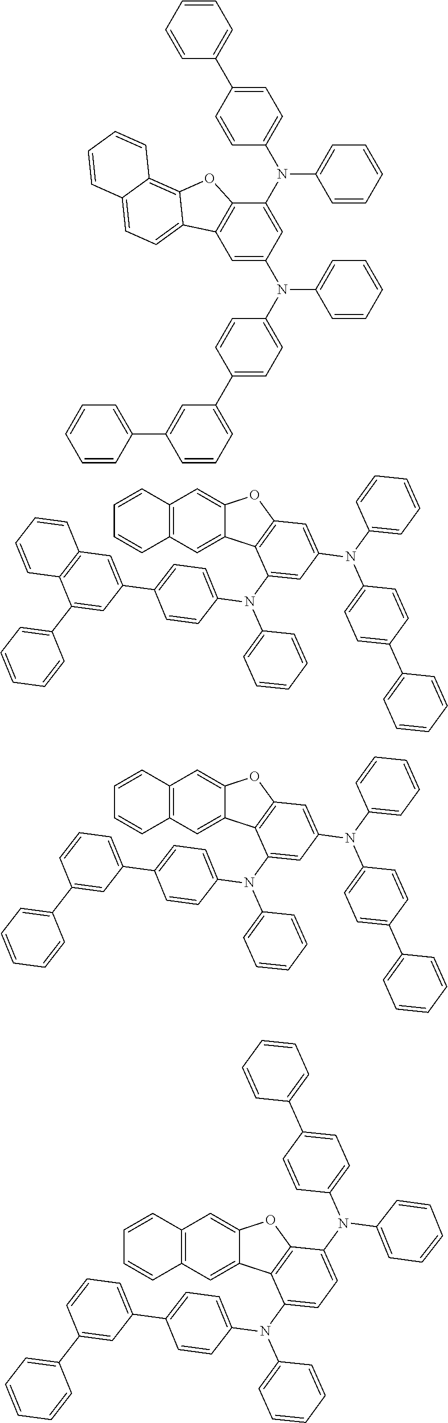 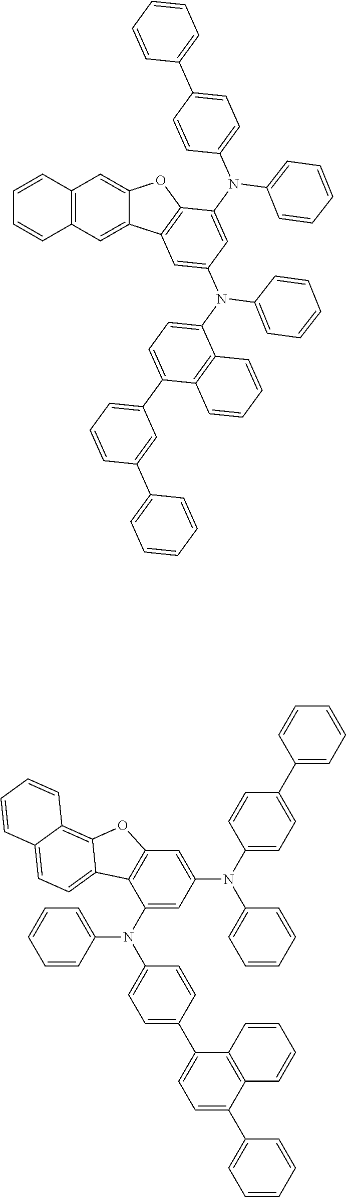 [SMILES: c1ccc(-c2ccc(N(c3ccccc3)c3cc(N(c4ccccc4)c4ccc(-c5cccc(-c6ccccc6)c5)c5ccccc45)cc4c3oc3cc5ccccc5cc34)cc2)cc1 ; c1ccc(-c2ccc(N(c3ccccc3)c3cc(N(c4ccccc4)c4ccc(-c5ccc(-c6ccccc6)c6ccccc56)cc4)c4c(c3)oc3c5ccccc5ccc34)cc2)cc1] 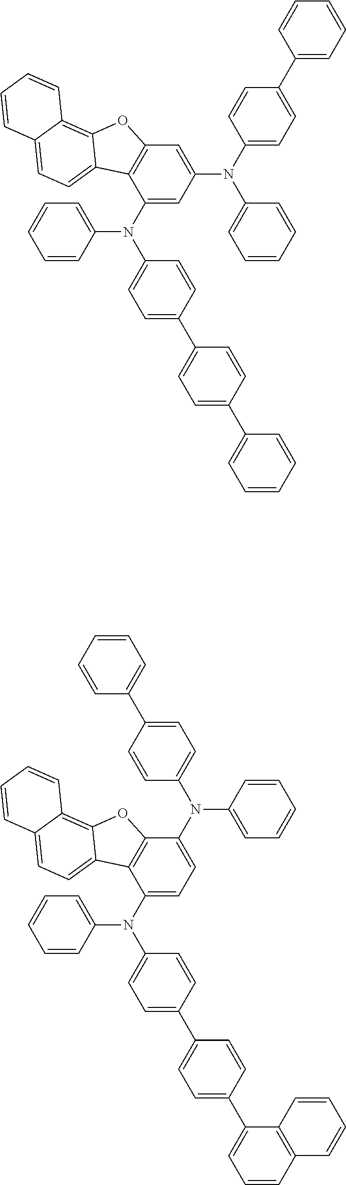 [SMILES: c1ccc(-c2ccc(-c3ccc(N(c4ccccc4)c4cc(N(c5ccccc5)c5ccc(-c6ccccc6)cc5)cc5oc6c7ccccc7ccc6c45)cc3)cc2)cc1 ; c1ccc(-c2ccc(N(c3ccccc3)c3ccc(N(c4ccccc4)c4ccc(-c5ccc(-c6cccc7ccccc67)cc5)cc4)c4c3oc3c5ccccc5ccc34)cc2)cc1] 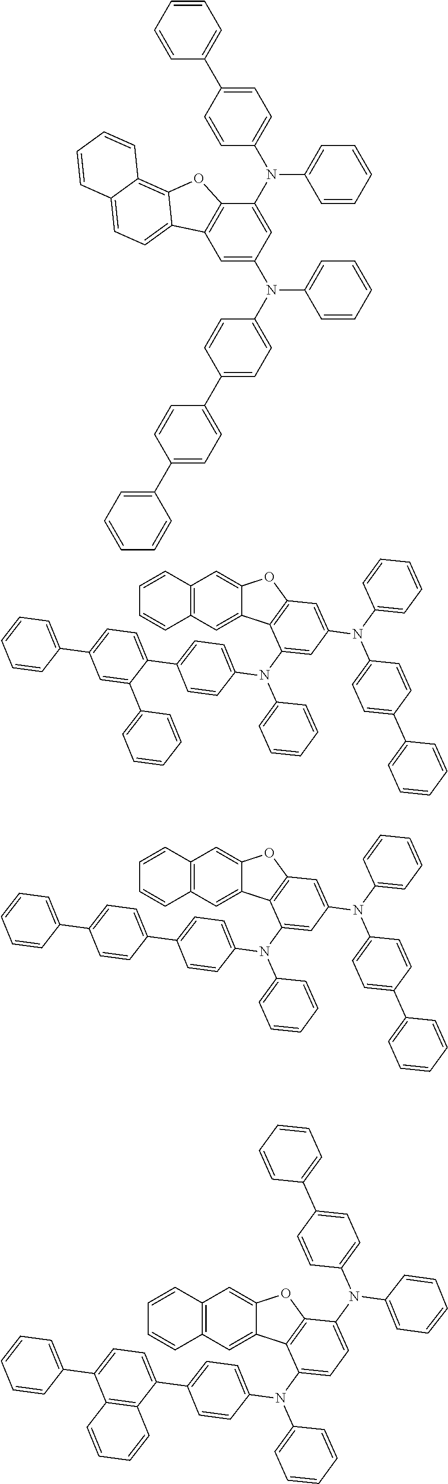 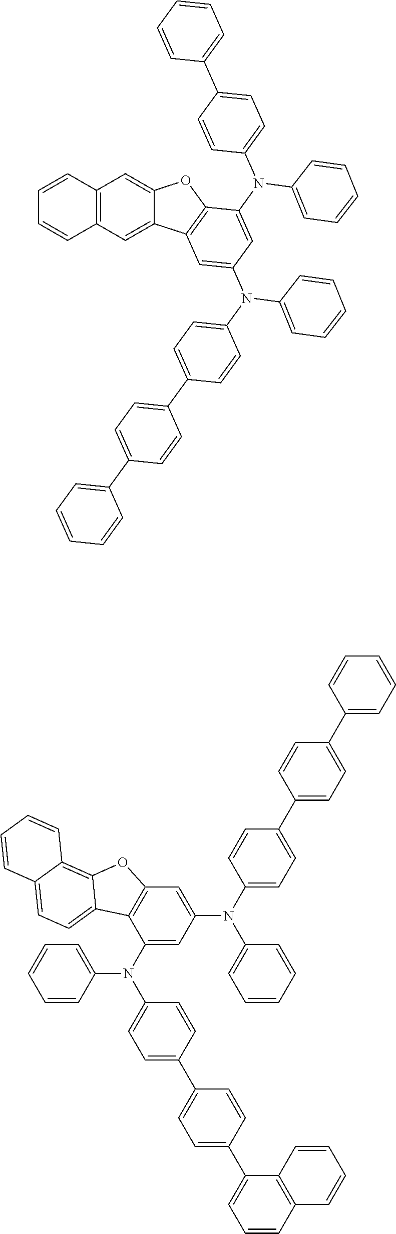 [SMILES: c1ccc(-c2ccc(-c3ccc(N(c4ccccc4)c4cc(N(c5ccccc5)c5ccc(-c6ccccc6)cc5)c5oc6cc7ccccc7cc6c5c4)cc3)cc2)cc1 ; c1ccc(-c2ccc(-c3ccc(N(c4ccccc4)c4cc(N(c5ccccc5)c5ccc(-c6ccc(-c7cccc8ccccc78)cc6)cc5)c5c(c4)oc4c6ccccc6ccc45)cc3)cc2)cc1] 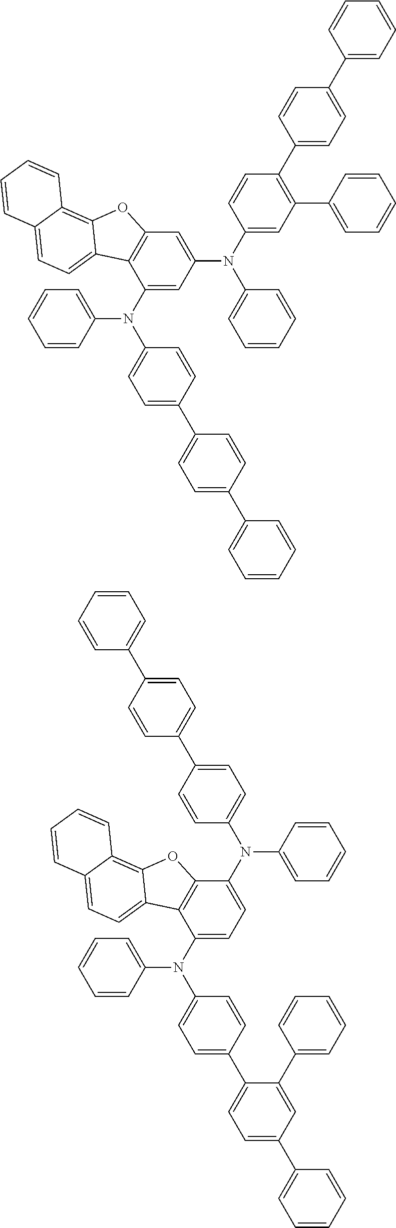 [SMILES: c1ccc(-c2ccc(-c3ccc(N(c4ccccc4)c4cc(N(c5ccccc5)c5ccc(-c6ccc(-c7ccccc7)cc6)c(-c6ccccc6)c5)cc5oc6c7ccccc7ccc6c45)cc3)cc2)cc1 ; c1ccc(-c2ccc(-c3ccc(N(c4ccccc4)c4ccc(N(c5ccccc5)c5ccc(-c6ccc(-c7ccccc7)cc6-c6ccccc6)cc5)c5c4oc4c6ccccc6ccc45)cc3)cc2)cc1] 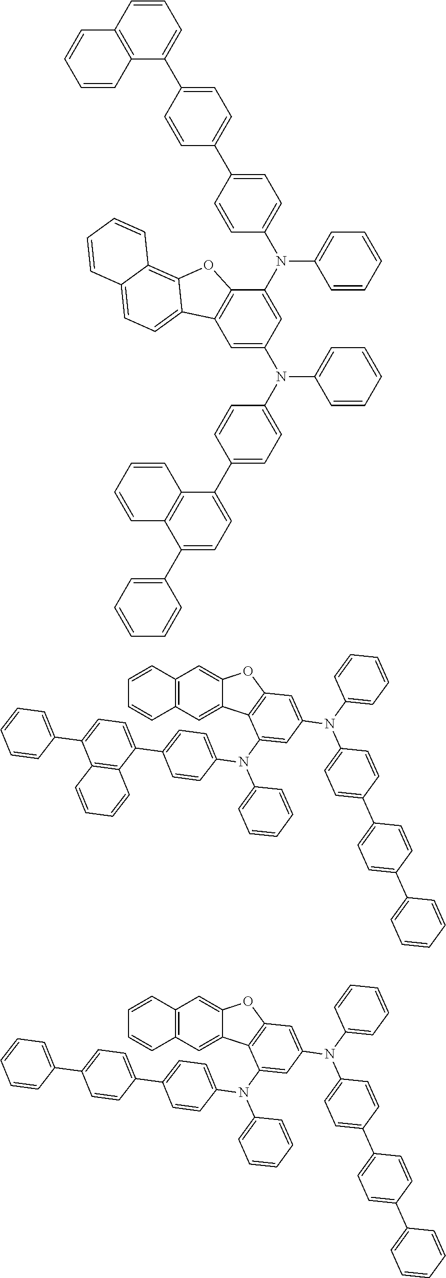 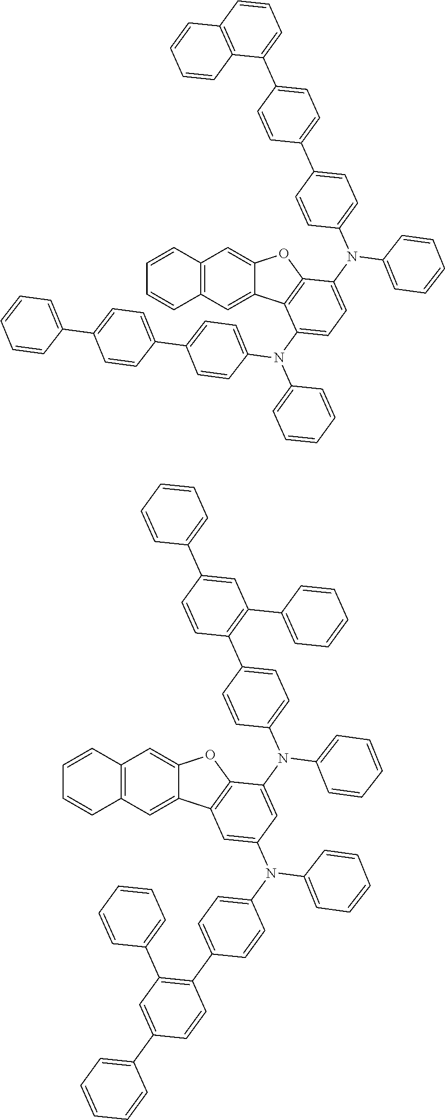 [SMILES: c1ccc(-c2ccc(-c3ccc(N(c4ccccc4)c4ccc(N(c5ccccc5)c5ccc(-c6ccc(-c7cccc8ccccc78)cc6)cc5)c5oc6cc7ccccc7cc6c45)cc3)cc2)cc1 ; c1ccc(-c2ccc(-c3ccc(N(c4ccccc4)c4cc(N(c5ccccc5)c5ccc(-c6ccc(-c7ccccc7)cc6-c6ccccc6)cc5)c5oc6cc7ccccc7cc6c5c4)cc3)c(-c3ccccc3)c2)cc1] 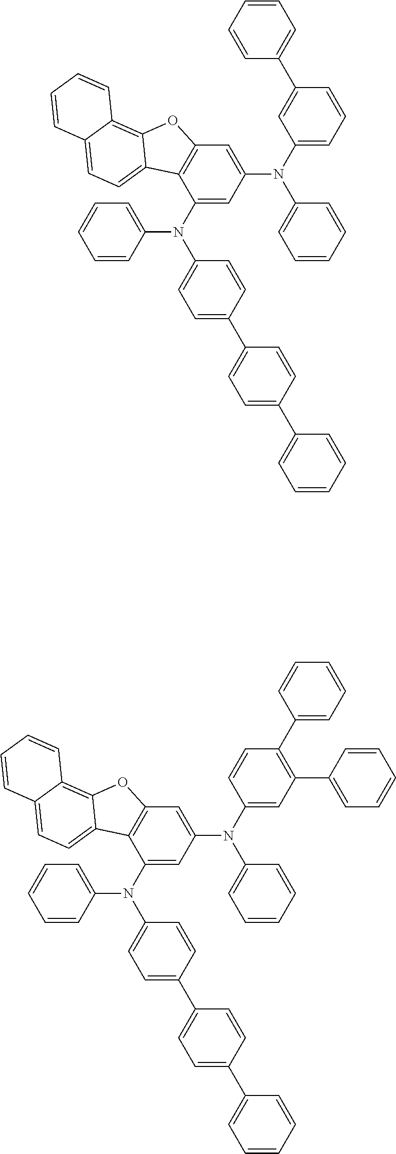 [SMILES: c1ccc(-c2ccc(-c3ccc(N(c4ccccc4)c4cc(N(c5ccccc5)c5cccc(-c6ccccc6)c5)cc5oc6c7ccccc7ccc6c45)cc3)cc2)cc1 ; c1ccc(-c2ccc(-c3ccc(N(c4ccccc4)c4cc(N(c5ccccc5)c5ccc(-c6ccccc6)c(-c6ccccc6)c5)cc5oc6c7ccccc7ccc6c45)cc3)cc2)cc1] 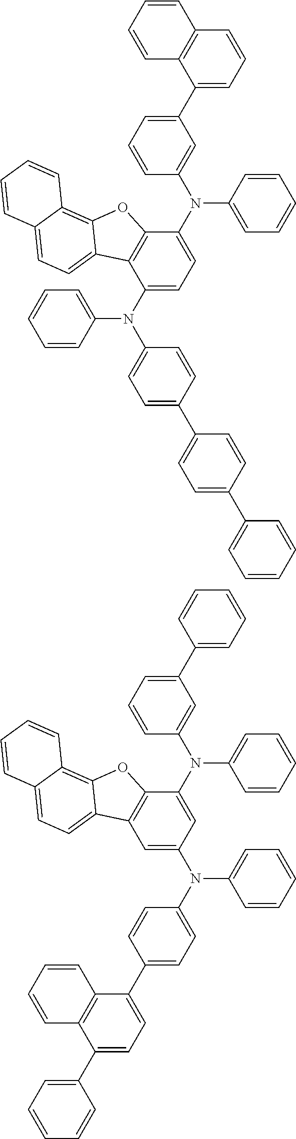 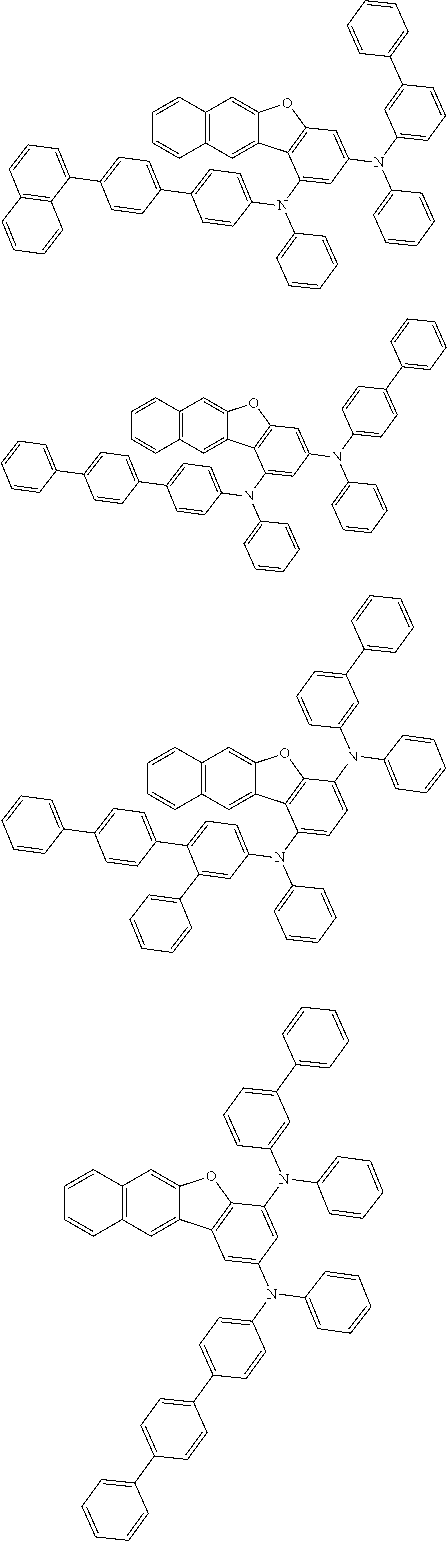 [SMILES: c1ccc(-c2cccc(N(c3ccccc3)c3cc(N(c4ccccc4)c4ccc(-c5ccc(-c6cccc7ccccc67)cc5)cc4)c4c(c3)oc3cc5ccccc5cc34)c2)cc1 ; c1ccc(-c2ccc(-c3ccc(N(c4ccccc4)c4cc(N(c5ccccc5)c5ccc(-c6ccccc6)cc5)cc5oc6cc7ccccc7cc6c45)cc3)cc2)cc1 ; c1ccc(-c2ccc(-c3ccc(N(c4ccccc4)c4ccc(N(c5ccccc5)c5cccc(-c6ccccc6)c5)c5oc6cc7ccccc7cc6c45)cc3-c3ccccc3)cc2)cc1 ; c1ccc(-c2ccc(-c3ccc(N(c4ccccc4)c4cc(N(c5ccccc5)c5cccc(-c6ccccc6)c5)c5oc6cc7ccccc7cc6c5c4)cc3)cc2)cc1] 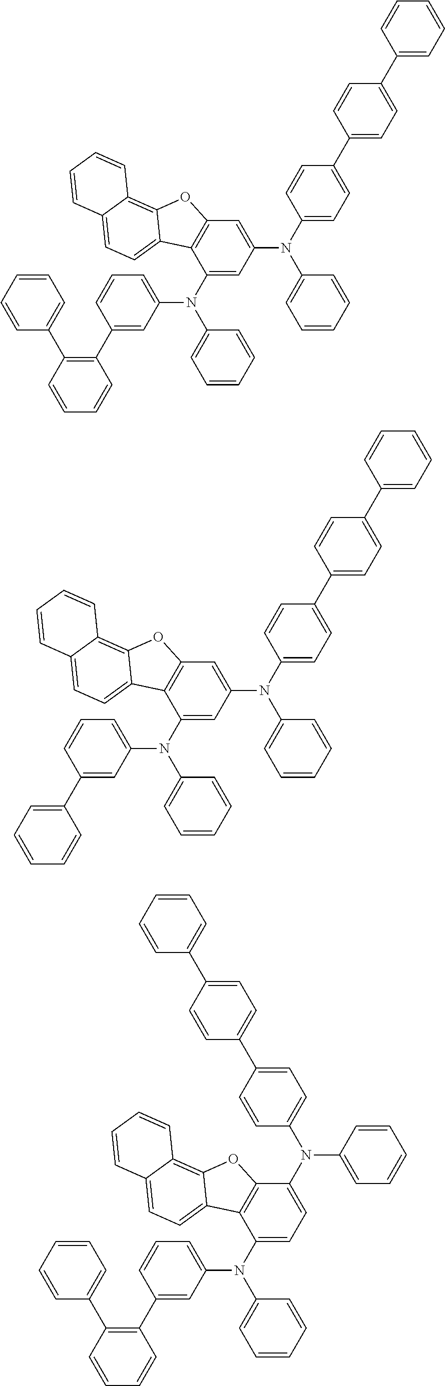 [SMILES: c1ccc(-c2ccc(-c3ccc(N(c4ccccc4)c4cc(N(c5ccccc5)c5cccc(-c6ccccc6-c6ccccc6)c5)c5c(c4)oc4c6ccccc6ccc45)cc3)cc2)cc1 ; c1ccc(-c2ccc(-c3ccc(N(c4ccccc4)c4cc(N(c5ccccc5)c5cccc(-c6ccccc6)c5)c5c(c4)oc4c6ccccc6ccc45)cc3)cc2)cc1 ; c1ccc(-c2ccc(-c3ccc(N(c4ccccc4)c4ccc(N(c5ccccc5)c5cccc(-c6ccccc6-c6ccccc6)c5)c5c4oc4c6ccccc6ccc45)cc3)cc2)cc1] 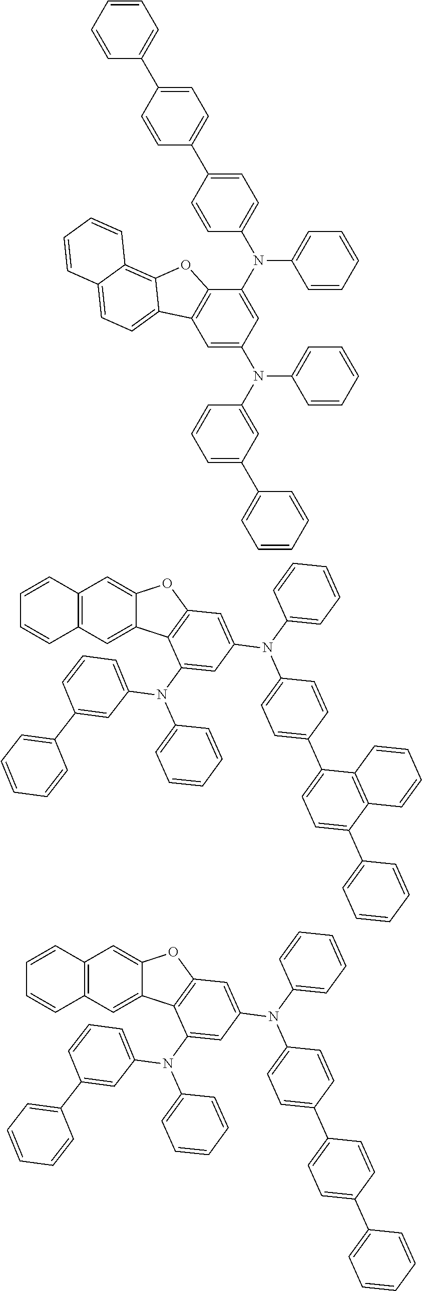 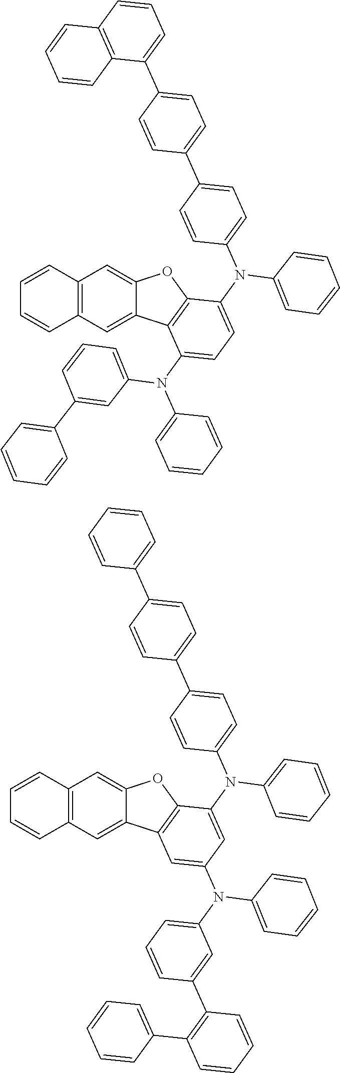 [SMILES: c1ccc(-c2cccc(N(c3ccccc3)c3ccc(N(c4ccccc4)c4ccc(-c5ccc(-c6cccc7ccccc67)cc5)cc4)c4oc5cc6ccccc6cc5c34)c2)cc1 ; c1ccc(-c2ccc(-c3ccc(N(c4ccccc4)c4cc(N(c5ccccc5)c5cccc(-c6ccccc6-c6ccccc6)c5)cc5c4oc4cc6ccccc6cc45)cc3)cc2)cc1] 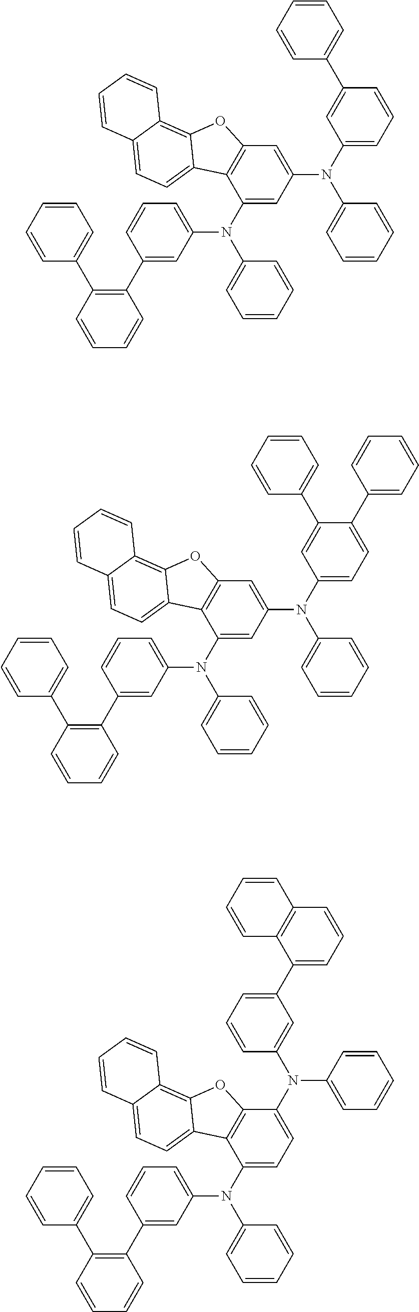 [SMILES: c1ccc(-c2cccc(N(c3ccccc3)c3cc(N(c4ccccc4)c4cccc(-c5ccccc5-c5ccccc5)c4)c4c(c3)oc3c5ccccc5ccc34)c2)cc1 ; c1ccc(-c2ccccc2-c2cccc(N(c3ccccc3)c3cc(N(c4ccccc4)c4ccc(-c5ccccc5)c(-c5ccccc5)c4)cc4oc5c6ccccc6ccc5c34)c2)cc1 ; c1ccc(-c2ccccc2-c2cccc(N(c3ccccc3)c3ccc(N(c4ccccc4)c4cccc(-c5cccc6ccccc56)c4)c4oc5c6ccccc6ccc5c34)c2)cc1] 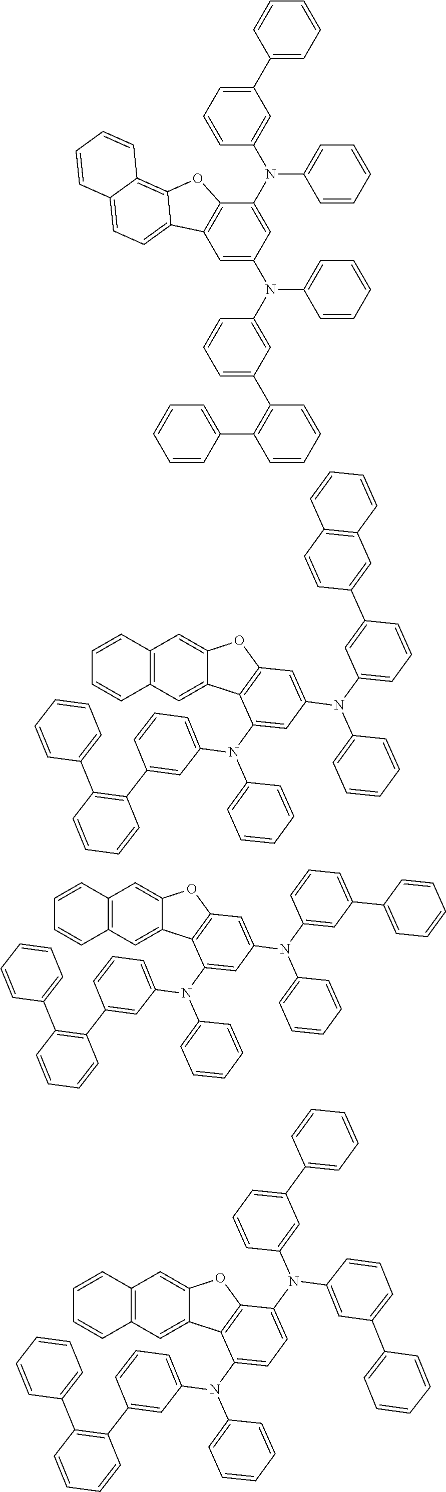 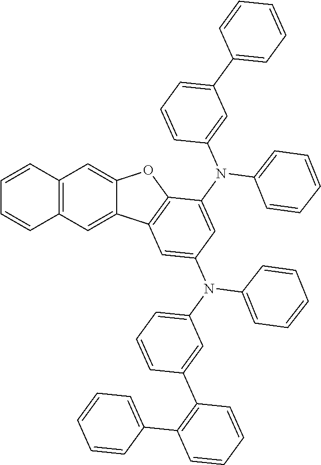 [SMILES: c1ccc(-c2cccc(N(c3ccccc3)c3cc(N(c4ccccc4)c4cccc(-c5ccccc5-c5ccccc5)c4)cc4c3oc3cc5ccccc5cc34)c2)cc1]
